(19) United States
(12) Patent Application Publication
(10) Pub. No.: US 2025/0121073 A1
(43) Pub. Date: Apr. 17, 2025

(54) IRAK DEGRADERS AND USES THEREOF

(71) Applicant: Kymera Therapeutics, Inc., Watertown, MA (US)

(72) Inventors: Nello Mainolfi, Watertown, MA (US); Nan Ji, Watertown, MA (US); Matthew M. Weiss, Watertown, MA (US); Xiaozhang Zheng, Watertown, MA (US); Yi Zhang, Watertown, MA (US); Paul R. Fleming, Watertown, MA (US); Xiao Zhu, Watertown, MA (US)

(21) Appl. No.: 18/912,154

(22) Filed: Oct. 10, 2024

Related U.S. Application Data
(62) Division of application No. 17/646,556, filed on Dec. 30, 2021, now Pat. No. 12,150,995.
(60) Provisional application No. 63/132,332, filed on Dec. 30, 2020.

(51) Int. Cl. A61K 47/55; A61K 47/54

(52) U.S. Cl. CPC A61K 47/55; A61K 47/545

(57) ABSTRACT

The present invention provides compounds, compositions thereof, and methods of using the same.

### CROSS-REFERENCE TO RELATED APPLICATIONS

This application is a divisional of U.S. application Ser. No. 17/646,556, filed Dec. 30, 2021, which claims the benefit of U.S. Provisional Appl. No. 63/132,332, filed Dec. 30, 2020, the entirety of which is herein incorporated by reference.

### TECHNICAL FIELD OF THE INVENTION

The present invention relates to compounds and methods useful for the modulation of one or more interleukin-1 receptor-associated kinases (“IRAK”) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

An ongoing need exists in the art for effective treatments for disease, especially hyperplasias and cancers, such as multiple myeloma. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins such as interleukin-1 receptor-associated kinases (“IRAK”) hold promise as therapeutic agents. Accordingly, there remains a need to find compounds that are IRAK degraders useful as therapeutic agents.

### SUMMARY OF THE INVENTION

The present application relates novel bifunctional compounds, which function to recruit IRAK kinases to E3 Ubiquitin Ligase for degradation, and methods of preparation and uses thereof. In particular, the present disclosure provides bifunctional compounds, which find utility as modulators of targeted ubiquitination of IRAK kinases, which are then degraded and/or otherwise inhibited by the bifunctional compounds as described herein. Also provided are monovalent compounds, which find utility as inducers of targeted ubiquitination of IRAK kinases, which are then degraded and/or otherwise inhibited by the monovalent compounds as described herein. An advantage of the compounds provided herein is that a broad range of pharmacological activities is possible, consistent with the degradation/inhibition of IRAK kinases. In addition, the description provides methods of using an effective amount of the compounds as described herein for the treatment or amelioration of a disease condition, such as cancer, e.g., multiple myeloma.

The present application further relates to targeted degradation of IRAK kinases through the use of bifunctional molecules, including bifunctional molecules that link a degradation inducing moiety to a ligand that binds IRAK kinases having the following general formula I:

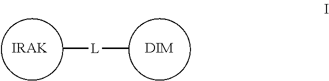

or a pharmaceutically acceptable salt thereof, wherein each variable is as defined and described herein.

It has now been found that compounds of this invention, and pharmaceutically acceptable compositions thereof, are effective for the modulation of targeted ubiquitination. Such compounds have the formula I-a:

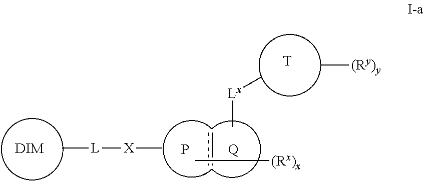

or a pharmaceutically acceptable salt thereof, wherein each variable is as defined and described herein.

The present invention further relates to bifunctional compounds that not only degrade IRAK, but also degrade IMiD substrates, such as Ikaros, Aiolos, or Ikaros and Aiolos.

Compounds of the present invention, and pharmaceutically acceptable compositions thereof, are useful for treating a variety of diseases, disorders or conditions, associated with regulation of signaling pathways implicating IRAK kinases. Such diseases, disorders, or conditions include those described herein.

Compounds provided by this invention are also useful for the study of IRAK enzymes in biological and pathological phenomena; the study of intracellular signal transduction pathways occurring in bodily tissues; and the comparative evaluation of new IRAK inhibitors or IRAK degraders or other regulators of kinases, signaling pathways, and cytokine levels in vitro or in vivo.

### DETAILED DESCRIPTION OF CERTAIN EMBODIMENTS

### 1. General Description of Certain Embodiments of the Invention

Compounds of the present invention, and compositions thereof, are useful as degraders and/or inhibitors of one or more IRAK protein kinases. In some embodiments, a provided compound degrades and/or inhibits IRAK-1/2/3/4. In some embodiments, a provided compound degrades IRAK4 and IMiD substrates, such as Ikaros, Aiolos, or Ikaros and Aiolos.

In certain embodiments, the present invention provides a compound of formula I:

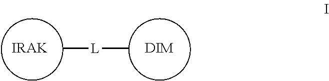

or a pharmaceutically acceptable salt thereof, wherein:
IRAK is an IRAK binding moiety capable of binding to one or more of IRAK-1, -2, -3, or -4;
L is a bivalent moiety that connects IRAK to DIM; and
DIM is a degradation inducing moiety.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Oiganic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

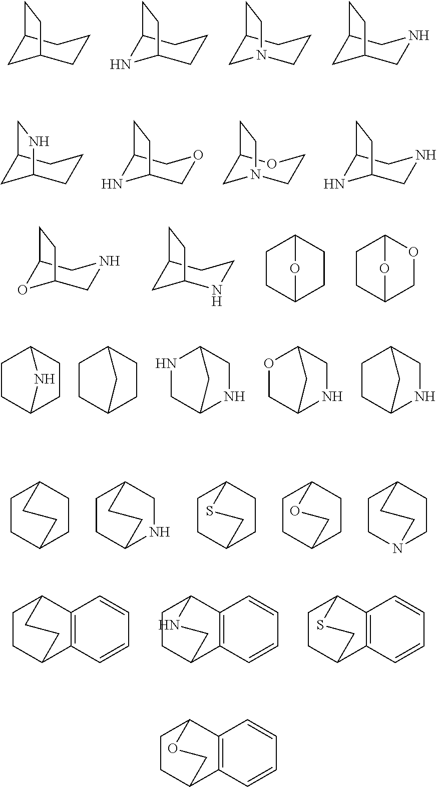

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

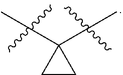 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, or spirocyclic. A heterocyclyl group may contain one or more oxo (C═O) or thioxo (S═O) group. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄(CH₂)₀₋₁Ph which may be substituted with R^{∘}; —CH═CHPh, which may be substituted with R^{∘}; —(CH₂)₀₋₄(CH₂)₀₋₁₋ pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄C(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄ OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄ S(O)₂R^{∘}; —(CH₂)₀₋₄S(O)₂OR^{∘}; —(CH₂)₀₋₄S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R*include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “provided compound” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “inhibitor” is defined as a compound that binds to and/or inhibits an IRAK kinase with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional or monovalent compound that binds to and/or inhibits both an IRAK kinase and an E3 ligase with measurable affinity resulting in the ubiquitination and subsequent degradation of the IRAK kinase. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in an IRAK protein kinase activity between a sample comprising a compound of the present invention, or composition thereof, and an IRAK protein kinase, and an equivalent sample comprising an IRAK protein kinase, in the absence of said compound, or composition thereof.

### 3. Description of Exemplary Embodiments

As described above, in certain embodiments, the present invention provides a compound of formula I:

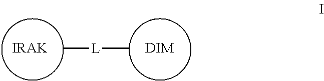

or a pharmaceutically acceptable salt thereof, wherein:
IRAK is an IRAK binding moiety capable of binding to one or more of IRAK-1, -2, -3, or -4;
L is a bivalent moiety that connects IRAK to DIM; and
DIM is a degradation inducing moiety.

In some embodiments, the present invention provides a compound of formula I:

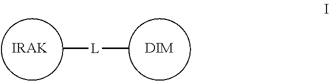

or a pharmaceutically acceptable salt thereof, wherein:
IRAK is an IRAK4 binding moiety;
L is a bivalent moiety that connects IRAK to DIM; and
DIM is an E3 ubiquitin ligase binding moiety (LBM), a lysine mimetic, or a hydrogen atom.

### IRAK Binding Moiety (IRAK)

In certain embodiments, the present invention provides a compound of formula I, where IRAK is a IRAK4 binding moiety thereby forming a compound of formulae I-a:

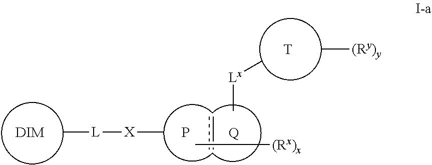

or a pharmaceutically acceptable salt thereof, wherein DIM and L are as defined and described herein, and wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R₂, or

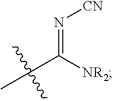

or
   two R^{x} groups are optionally taken together to form an optionally substituted 5-6 membered partially unsaturated or aryl fused ring having 0-3 heteroatoms independently selected from nitrogen, oxygen, or sulfur, or a 3-5 membered saturated or partially unsaturated carbocyclic or heterocyclic spiro fused ring having 0-3 heteroatoms independently selected from nitrogen, oxygen, or sulfur,
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or

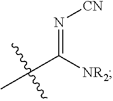

or
   a single R^{y} and a single R^{x} are optionally taken together with their intervening atoms to form a 8-20 membered saturated or partially unsaturated carbocyclic or heterocyclic monocyclic or bicyclic ring having 1-10 heteroatoms, independently selected from nitrogen, oxygen, and sulfur;
each R^{z} is independently an optionally substituted group selected from C₁₋₆ aliphatic, phenyl, a 4-9 membered saturated or partially unsaturated carbocyclic or heterocyclic monocyclic, bicyclic, bridged bicyclic, or spirocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring P and Ring Q are optionally fused rings independently selected from phenyl or benzo, a 4-7 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring P and Ring Q are independently and optionally substituted with 1-2 oxo groups;
Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups;
L^{x} is a covalent bond or a C₁₋₃ bivalent straight or branched saturated or unsaturated hydrocarbon chain wherein 1-2 methylene units of the chain are independently and optionally replaced with -Cy^{x}-, —O—, —S—, —C(O)—, —C(S)—, —CR₂—, —CRF—, —CF₂—, —NR—, —N═CR—, —CR═CR—, or —S(O)₂—, wherein R of —CR₂—, —CRF—, —NR—, —N═CR—, or —CR═CR— can combine with R^{x} or R^{y} to form a 4-7 membered saturated or partially unsaturated carbocyclic or heterocyclic ring having 1-3 heteroatoms independently selected from nitrogen, oxygen, and sulfur;
-Cy^{x}- is an optionally substituted ring selected from a 3-5 membered saturated or partially unsaturated carbocyclic or heterocyclic ring having 0-3 heteroatoms independently selected from nitrogen, oxygen, and sulfur, or a 5 membered heteroaryl ring having 1-4 heteroatoms independently selected from nitrogen, oxygen, and sulfur, wherein -Cy^{x}- is optionally substituted with 1-2 oxo groups;
X is a covalent bond or a 4-6 membered saturated or partially unsaturated carbocyclic or heterocyclic ring having 1-2 heteroatoms independently selected from nitrogen, oxygen, and sulfur;
[symbol] is a single or double bond;
each x is 0, 1, 2, 3 or 4; and
each y is 0, 1, 2, 3 or 4.

As described herein, a core structure depicted as

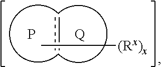

includes for example, structures

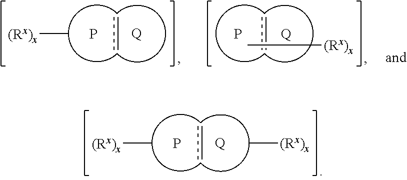

As defined generally above, each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R², —SiR₃, —Si(OR)R₂, or

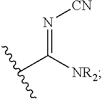

or two R^{x} groups are optionally taken together to form an optionally substituted 5-6 membered partially unsaturated or aryl fused ring having 0-3 heteroatoms independently selected from nitrogen, oxygen, or sulfur, or a 3-5 membered saturated or partially unsaturated carbocyclic or heterocyclic spiro fused ring having 0-3 heteroatoms independently selected from nitrogen, oxygen, or sulfur.

In some embodiments, R^{x} is hydrogen. In some embodiments, R^{x} is deuterium. In some embodiments, R^{x} is R^{z}. In some embodiments, R^{x} is halogen. In some embodiments, R^{x} is —CN. In some embodiments, each R^{x} is independently —NO₂. In some embodiments, R^{x} is —OR. In some embodiments, each R^{x} is independently —SR. In some embodiments, R^{x} is —NR₂. In some embodiments, R^{x} is —S(O)₂R. In some embodiments, R^{x} is —S(O)₂NR₂. In some embodiments, R^{x} is —S(O)R. In some embodiments, R^{x} is —CFR₂. In some embodiments, R^{x} is —CF₂R. In some embodiments, R^{x} is —CF₃. In some embodiments, R^{x} is —CR₂(OR). In some embodiments, R^{x} is —CR₂(NR₂). In some embodiments, R^{x} is —C(O)R. In some embodiments, R^{x} is —C(O)OR. In some embodiments, R^{x} is —C(O)NR₂. In some embodiments, eR^{x} is —N⁺(O⁻)R₂. In some embodiments, R^{x} is —OP(O)R₂. In some embodiments, R^{x} is —OP(O)(OR)₂. In some embodiments, R^{x} is —OP(O)(OR)NR₂. In some embodiments, R^{x} is —OP(O)(NR₂)₂. In some embodiments R^{x} is —P(O)R₂. In some embodiments, R^{x} is —SiR₃. In some embodiments, R^{x} is —Si(OR)R². In some embodiments, R^{x} is —SF₅. In some embodiments, R^{x} is

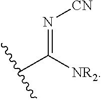

In some embodiments, two R^{x} groups are optionally taken together to form an optionally substituted 5-6 membered partially unsaturated or aryl fused ring having 0-3 heteroatoms independently selected from nitrogen, oxygen, or sulfur. In some embodiments, two R^{x} groups are optionally taken together to form an optionally substituted 3-5 membered saturated or partially unsaturated carbocyclic or heterocyclic spiro fused ring having 0-3 heteroatoms independently selected from nitrogen, oxygen, or sulfur,

In some embodiments, R^{x} is

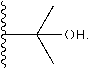 [SMILES: *C(C)(C)O]

In some embodiments, R^{x} is fluoro. In some embodiments, R^{x} is chloro. In some embodiments, R^{x} is —CF₂H. In some embodiments, R^{x} is —OMe. In some embodiments, R^{x} is —OEt. In some embodiments, R^{x} is -OiPr. In some embodiments, R^{x} is —NMe₂. In some embodiments, R^{x} is —SMe. In some embodiments, R^{x} is —S(O)Me. In some embodiments, R^{x} is —S(O)₂Me. In some embodiments, R^{x} is -Me. In some embodiments, R^{x} is -Et. In some embodiments, R^{x} is -iPr. In some embodiments, R^{x} is cyclopropyl. In some embodiments, R^{x} is -Ac. In some embodiments, R^{x} is —CO₂Me. In some embodiments, R^{x} is —CO₂H. In some embodiments, R^{x} is —OCF₂H. In some embodiments, R^{x} is —OCF₃. In some embodiments, R^{x} is

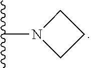 [SMILES: *N1CCC1]

In some embodiments, R^{x} is

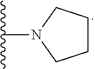 [SMILES: *N1CCCC1]

In some embodiments, R^{x} is

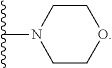 [SMILES: *N1CCOCC1]

In some embodiments, R^{x} is

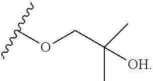 [SMILES: *OCC(C)(C)O]

In some embodiments, R^{x} is

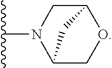 [SMILES: *N1C[C@@H]2C[C@H]1CO2]

In some embodiments, R^{x} is

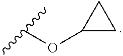 [SMILES: *OC1CC1]

In some embodiments, R^{x} is

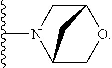 [SMILES: *N1C[C@H]2C[C@@H]1CO2]

In some embodiments, R^{x} is

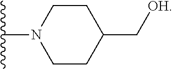 [SMILES: *N1CCC(CO)CC1]

In some embodiments, R^{x} is

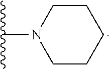 [SMILES: *N1CCCCC1]

In some embodiments, R^{x} is

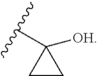 [SMILES: *C1(O)CC1]

In some embodiments, R^{x} is

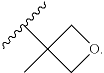 [SMILES: *C1(C)COC1]

In some embodiments, R^{x} is

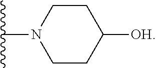 [SMILES: *N1CCC(O)CC1]

In some embodiments, R^{x} is

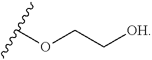 [SMILES: *OCCO]

In some embodiments, R^{x} is

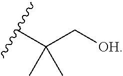 [SMILES: *C(C)(C)CO]

In some embodiments, R^{x} is

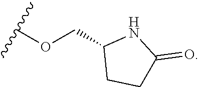 [SMILES: *OC[C@H]1CCC(=O)N1]

In some embodiments, R is

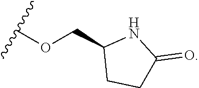 [SMILES: *OC[C@@H]1CCC(=O)N1]

In some embodiments, R^{x} is

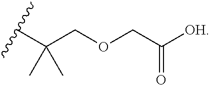 [SMILES: *C(C)(C)COCC(=O)O]

In some embodiments, R^{x} is

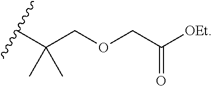 [SMILES: *C(C)(C)COCC(=O)OCC]

In some embodiments, R^{x} is

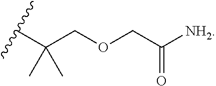 [SMILES: *C(C)(C)COCC(N)=O]

In some embodiments, each R^{x} is selected from those depicted in Table 1, below.

As generally defined above, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atom to form a 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur.

In some embodiments, each R is independently hydrogen. In some embodiments, each R is an optionally substituted group selected from C₁₋₆ aliphatic. In some embodiments, each R is an optionally substituted phenyl. In some embodiments, each R is an optionally substituted 4-7 membered saturated or partially unsaturated heterocyclic having 1-2 heteroatoms independently selected from nitrogen, oxygen, and sulfur. In some embodiments, each R is an optionally substituted 5-6 membered heteroaryl ring having 1-4 heteroatoms independently selected from nitrogen, oxygen, and sulfur. In some embodiments, two R groups on the same atom are optionally taken together with their intervening atom to form a 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur.

In some embodiments, each R is selected from those depicted in Table 1, below.

As defined generally above, each R^{y} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R₂, —SFS, or

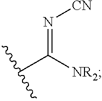

or two R^{y} groups are optionally taken together to form an optionally substituted 5-6 membered partially unsaturated or aryl fused ring having 0-3 heteroatoms independently selected from nitrogen, oxygen, or sulfur; or a single R^{y} and a single R^{x} are optionally taken together with their intervening atoms to form a 8-20 membered saturated or partially unsaturated carbocyclic or heterocyclic monocyclic or bicyclic ring having 1-10 heteroatoms, independently selected from nitrogen, oxygen, and sulfur.

In some embodiments, R^{y} is hydrogen. In some embodiments, R^{y} is deuterium. In some embodiments, R^{y} is R^{z}. In some embodiments, R^{y} is halogen. In some embodiments, R^{y} is —CN. In some embodiments, R^{y} is —NO₂. In some embodiments, R^{y} is —OR. In some embodiments, R^{y} is —SR. In some embodiments, R^{y} is —NR₂. In some embodiments, R^{y} is —S(O)₂R. In some embodiments, R^{y} is —S(O)₂NR₂. In some embodiments, R^{y} is —S(O)R. In some embodiments, R^{y} is —CFR₂. In some embodiments, R^{y} is —CF₂R. In some embodiments, R^{y} is —CF₃. In some embodiments, R^{y} is —CR₂(OR). In some embodiments, R^{y} is —CR₂(NR₂). In some embodiments, R^{y} is —C(O)R. In some embodiments, R^{y} is —C(O)OR. In some embodiments, R^{y} is —C(O)NR₂. In some embodiments, R^{y} is —N⁺(O⁻)R₂. In some embodiments, R^{y} is —OP(O)R₂. In some embodiments, R^{y} is —OP(O)(OR)₂. In some embodiments, R^{y} is —OP(O)(OR)NR₂. In some embodiments, R^{y} is —OP(O)(NR₂)₂. In some embodiments, R^{y} is —P(O)R₂. In some embodiments, R^{y} is —SiR₃. In some embodiments, R^{y} is —Si(OR)R². In some embodiments, R is —SF₅. In some embodiments, R^{y} is

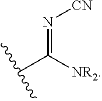

In some embodiments, two R groups are optionally taken together to form an optionally substituted 5-6 membered partially unsaturated or aryl fused ring having 0-3 heteroatoms independently selected from nitrogen, oxygen, or sulfur. In some embodiments, a single R^{y} and a single R^{x} are optionally taken together with their intervening atoms to form a 8-20 membered saturated or partially unsaturated carbocyclic or heterocyclic monocyclic or bicyclic ring having 1-10 heteroatoms, independently selected from nitrogen, oxygen, and sulfur.

In some embodiments, R^{y} is fluoro. In some embodiments, R^{y} is chloro. In some embodiments, R^{y} is —CN. In some embodiments, R^{y} is —CF₂Me. In some embodiments, R^{y} is —CFMe₂. In some embodiments, R^{y} is -Me. In some embodiments, R^{y} is —OMe. In some embodiments, R^{y} is —OCF₃. In some embodiments, R^{y} is fluoro. In some embodiments, R^{y} is cyclopropyl. In some embodiments, R^{y} is

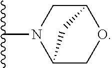 [SMILES: *N1C[C@@H]2C[C@H]1CO2]

In some embodiments, R^{y} is

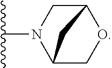 [SMILES: *N1C[C@H]2C[C@@H]1CO2]

In some embodiments, R^{y} and R^{x} taken together is

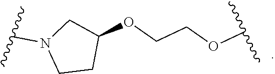 [SMILES: *OCCO[C@H]1CCN(*)C1]

In some embodiments, R^{y} and R^{x} taken together is

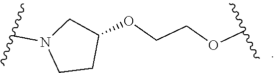 [SMILES: *OCCO[C@@H]1CCN(*)C1]

In some embodiments, R^{y} and R^{x} taken together is

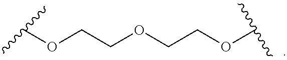 [SMILES: *OCCOCCO*]

In some embodiments, R^{y} and R^{x} taken together is

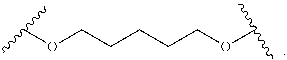 [SMILES: *OCCCCCO*]

In some embodiments, R^{y} and R^{x} taken together is

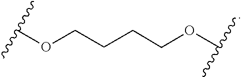 [SMILES: *OCCCCO*]

In some embodiments, each R^{y} is selected from those depicted in Table 1, below.

As generally defined above, each R^{z} is independently an optionally substituted group selected from C₁₋₆ aliphatic, phenyl, a 4-9 membered saturated or partially unsaturated carbocyclic or heterocyclic monocyclic, bicyclic, bridged bicyclic, or spirocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{z} is an optionally substituted group selected from C₁₋₆ aliphatic. In some embodiments, R^{z} is an optionally substituted phenyl. In some embodiments, R^{z} is an optionally substituted 4-9 membered saturated or partially unsaturated carbocyclic or heterocyclic monocyclic, bicyclic, bridged bicyclic, or spirocyclic ring having 1-2 heteroatoms independently selected from nitrogen, oxygen, and sulfur. In some embodiments, R^{z} is an optionally substituted 5-6 membered heteroaryl ring having 1-4 heteroatoms independently selected from nitrogen, oxygen, and sulfur.

In some embodiments, each R^{z} is selected from those depicted in Table 1, below.

As generally defined above, Ring P and Ring Q are optionally fused rings independently selected from phenyl or benzo, a 4-7 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring P and Ring Q are independently and optionally substituted with 1-2 oxo groups.

In some embodiments, Ring P phenyl or benzo. In some embodiments, Ring P is a 4-7 membered saturated or partially unsaturated carbocyclic or heterocyclic ring having 1-3 heteroatoms independently selected from nitrogen, oxygen, and sulfur. In some embodiments, Ring P is a 5-6 membered heteroaryl ring having 1-4 heteroatoms independently selected from nitrogen, oxygen, and sulfur. In some embodiments, Ring P is optionally substituted with 1-2 oxo groups. In some embodiments, Ring Q is phenyl or benzo. In some embodiments, Ring Q is a 4-7 membered saturated or partially unsaturated carbocyclic or heterocyclic ring having 1-3 heteroatoms independently selected from nitrogen, oxygen, and sulfur. In some embodiments, Ring Q is a 5-6 membered heteroaryl ring having 1-4 heteroatoms independently selected from nitrogen, oxygen, and sulfur. In some embodiments, Ring Q is optionally substituted with 1-2 oxo groups.

In some embodiments, Ring P and Ring Q are

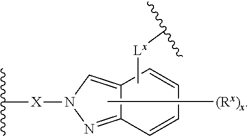

In some embodiments, Ring P and Ring Q are

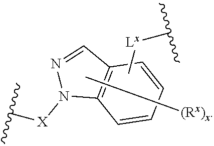

In some embodiments, Ring P and Ring Q are

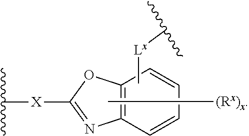

In some embodiments, Ring P and Ring Q are

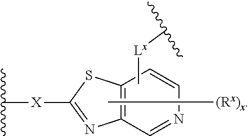

In some embodiments, Ring P and Ring Q are

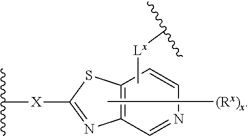

In some embodiments, Ring P and Ring Q are

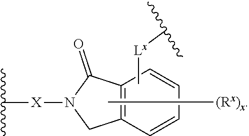

In some embodiments, Ring P and Ring Q are

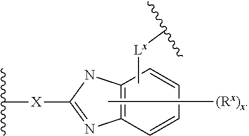

In some embodiments, Ring P and Ring Q are

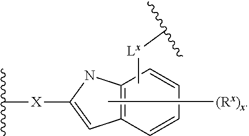

In some embodiments, Ring P and Ring Q are

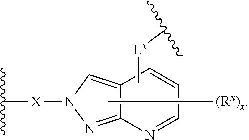

In some embodiments, Ring P and Ring Q are

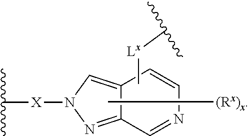

In some embodiments, Ring P and Ring Q are

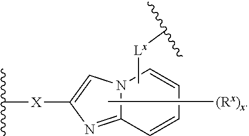

In some embodiments, Ring P and Ring Q are

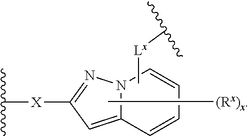

In some embodiments, Ring P and Ring Q are selected from those depicted in Table 1, below.

As generally defined above, Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups.

In some embodiments, Ring T is from phenyl. In some embodiments, Ring T is a 4-7 membered saturated or partially unsaturated carbocyclic or heterocyclic ring having 1-3 heteroatoms independently selected from nitrogen, oxygen, and sulfur. In some embodiments, Ring T is a 5-9 membered mono- or bicyclic heteroaryl ring having 1-4 heteroatoms independently selected from nitrogen, oxygen, and sulfur. In some embodiments, Ring T is further optionally substituted with 1-2 oxo groups.

In some embodiments, Ring T is

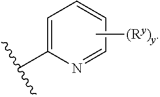

In some embodiments, Ring T is

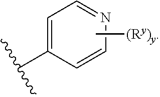

In some embodiments, Ring T is

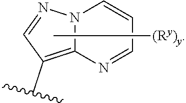

In some embodiments, Ring T is

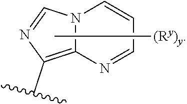

In some embodiments, Ring T is

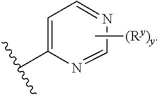

In some embodiments, Ring T is

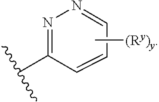

In some embodiments, Ring T is phenyl. In some embodiments, Ring T is

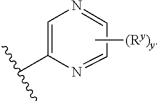

In some embodiments, Ring T is

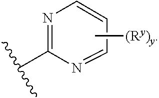

In some embodiments, Ring T is

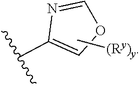

In some embodiments, Ring T is

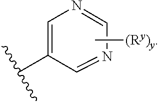

In some embodiments, Ring T is

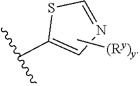

In some embodiments, Ring T is

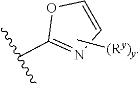

In some embodiments, Ring T is

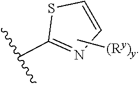

In some embodiments, Ring T is

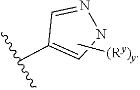

In some embodiments, Ring T is selected from those depicted in Table 1, below.

As generally defined above, L^{x} is a covalent bond or a C₁₋₃ bivalent straight or branched saturated or unsaturated hydrocarbon chain wherein 1-2 methylene units of the chain are independently and optionally replaced with -Cy^{x}-, —O—, —S—, —C(O)—, —C(S)—, —CR₂—, —CRF—, —CF₂—, —NR—, —N═CR—, —CR═CR—, or —S(O)₂—, wherein R of —CR₂—, —CRF—, —NR—, —N═CR—, or —CR═CR— can combine with R^{x} or R^{y} to form a 4-7 membered saturated or partially unsaturated carbocyclic or heterocyclic ring having 1-3 heteroatoms independently selected from nitrogen, oxygen, and sulfur.

In some embodiments, L^{x} is a covalent bond. In some embodiments, L^{x} is a C₁₋₃ bivalent straight or branched saturated or unsaturated hydrocarbon chain wherein 1-2 methylene units of the chain are independently and optionally replaced with -Cy^{x}-, —O—, —S—, —C(O)—, —C(S)—, —CR₂—, —CRF—, —CF₂—, —NR—, —N═CR—, —CR═CR—, or —S(O)₂—. In some embodiments, R of —CR₂—, —CRF—, —NR—, —N═CR—, or —CR═CR— can combine with R^{x} or R^{y} to form a 4-7 membered saturated or partially unsaturated carbocyclic or heterocyclic ring having 1-3 heteroatoms independently selected from nitrogen, oxygen, and sulfur.

In some embodiments, Ring L^{x} is —C(O)N(H)—. In some embodiments, Ring L^{x} is —CH₂C(O)N(H)—. In some embodiments, Ring L^{x} is

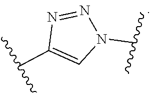 [SMILES: *c1cn(*)nn1]

In some embodiments, L^{x} combines with R^{y} to form

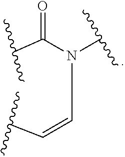 [SMILES: */C=C\N(*)C(*)=O]

In some embodiments, L^{x} combines with Ry to form

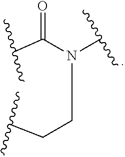 [SMILES: *CCN(*)C(*)=O]

In some embodiments, Ring L^{x} is selected from those depicted in Table 1, below.

As generally defined above, -Cy^{x}- is an optionally substituted ring selected from a 3-5 membered saturated or partially unsaturated carbocyclic or heterocyclic ring having 0-3 heteroatoms independently selected from nitrogen, oxygen, and sulfur, or a 5 membered heteroaryl ring having 1-4 heteroatoms independently selected from nitrogen, oxygen, and sulfur, wherein -Cy^{x}- is optionally substituted with 1-2 oxo groups.

In some embodiments, -Cy^{x}- is an optionally substituted ring selected from a 3-5 membered saturated or partially unsaturated carbocyclic or heterocyclic ring having 0-3 heteroatoms independently selected from nitrogen, oxygen, and sulfur. In some embodiments, -Cy^{x}- is a 5 membered heteroaryl ring having 1-4 heteroatoms independently selected from nitrogen, oxygen, and sulfur. In some embodiments, -Cy^{x}- is optionally substituted with 1-2 oxo groups.

In some embodiments, Ring -Cy^{x}- is selected from those depicted in Table 1, below.

As described above, X is a covalent bond or a 4-6 membered saturated or partially unsaturated carbocyclic or heterocyclic ring having 1-2 heteroatoms independently selected from nitrogen, oxygen, and sulfur.

In some embodiments, X is a covalent bond. In some embodiments, X is a 4-6 membered saturated or partially unsaturated carbocyclic or heterocyclic ring having 1-2 heteroatoms independently selected from nitrogen, oxygen, and sulfur.

In some embodiments, X is

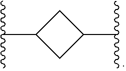 [SMILES: *C1CC(*)C1]

In some embodiments, X is

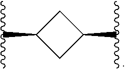 [SMILES: *[C@H]1C[C@@H](*)C1]

In some embodiments, X is

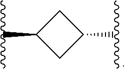 [SMILES: *[C@H]1C[C@H](*)C1]

In some embodiments, X is

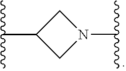 [SMILES: *C1CN(*)C1]

In some embodiments, X is

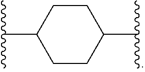 [SMILES: *C1CCC(*)CC1]

In some embodiments, X is

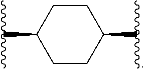 [SMILES: *[C@H]1CC[C@@H](*)CC1]

In some embodiments, X is

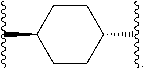 [SMILES: *[C@H]1CC[C@H](*)CC1]

In some embodiments, X is

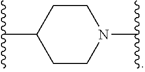 [SMILES: *C1CCN(*)CC1]

In some embodiments, X is selected from those depicted in Table 1, below.

As generally defined above [symbol] is a single or double bond.

In some embodiments, [symbol] is a single. In some embodiments, [symbol] is a double bond.

In some embodiments, Ring [symbol] is selected from those depicted in Table 1, below.

As generally defined above, each x and y are independently 0, 1, 2, 3 or 4.

In some embodiments, each x and y are independently 0. In some embodiments, each x and y are independently 1. In some embodiments, each x and y are independently 2. In some embodiments, each x and y are independently 3. In some embodiments, each x and y are independently 4.

In some embodiments, each x and y are selected from those depicted in Table 1, below.

In some embodiments, the present invention provides a compound of formula I-a, wherein Ring P and Ring Q form an indazole ring as shown, to provide a compound of formula I-d-1:

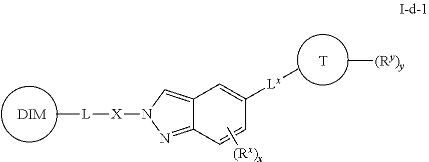

or a pharmaceutically acceptable salt thereof, wherein each of DIM, L^{x}, LU, X, R^{x}, R^{y}, Ring T, x, and y is as defined above and described in embodiments herein, both singly and in combination.

In some embodiments, the present invention provides a compound of formula I-a, wherein Ring P and Ring Q form a 6-azaindazole ring as shown, to provide a compound of formula I-d-2:

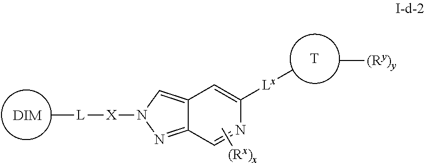

or a pharmaceutically acceptable salt thereof, wherein each of DIM, L, L^{x}, X, R^{x}, R^{y}, Ring T, x, and y is as defined above and described in embodiments herein, both singly and in combination.

In some embodiments, the present invention provides a compound of formula I-a, wherein Ring P and Ring Q form an indazole ring as shown, to provide a compound of formula I-d-3:

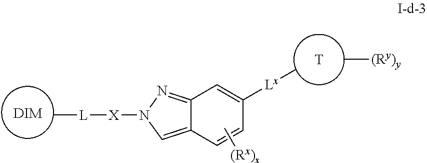

or a pharmaceutically acceptable salt thereof, wherein each of DIM, L, L^{x}, X, R^{x}, R^{y}, Ring T, x, and y is as defined above and described in embodiments herein, both singly and in combination.

In some embodiments, the present invention provides a compound of formula I-a, wherein Ring P and Ring Q form a pyrazolopyridine ring as shown, to provide a compound of formula I-d-4:

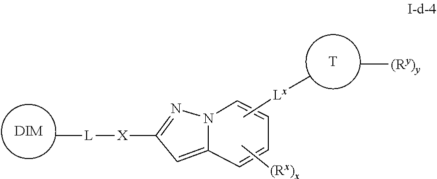

or a pharmaceutically acceptable salt thereof, wherein each of DIM, L, L^{x}, X, R^{x}, R^{y}, Ring T, x, and y is as defined above and described in embodiments herein, both singly and in combination.

In some embodiments, the present invention provides a compound of formula I-a, wherein Ring P and Ring Q form an imidazo[1,2-a]pyridine ring as shown, to provide a compound of formula I-d-5:

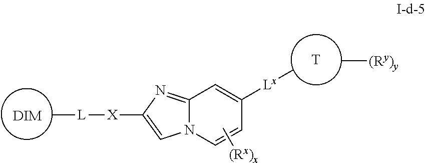

or a pharmaceutically acceptable salt thereof, wherein each of DIM, L, L^{x}, X, R^{x}, R^{y}, Ring T, x, and y is as defined above and described in embodiments herein, both singly and in combination.

In some embodiments, the present invention provides a compound of formula I-a, wherein Ring Q is benzo, a single R^{x} is —OR, and X is cyclohexyl as shown, to provide a compound of formula I-d-6:

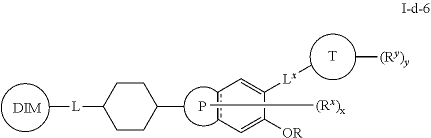

or a pharmaceutically acceptable salt thereof, wherein each of DIM, L, L^{x}, R^{x}, R^{y}, Ring P, Ring T, x, and y is as defined above and described in embodiments herein, both singly and in combination.

In some embodiments, the present invention provides a compound of formula I-a, wherein Ring P and Ring Q form an indazole ring, a single R^{x} is —OR, and X is cyclohexyl as shown, to provide a compound of formula I-d-7:

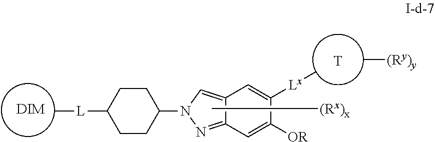

or a pharmaceutically acceptable salt thereof, wherein each of DIM, L, L^{x}, R^{x}, R^{y}, Ring T, x, and y is as defined above and described in embodiments herein, both singly and in combination.

In some embodiments, IRAK is

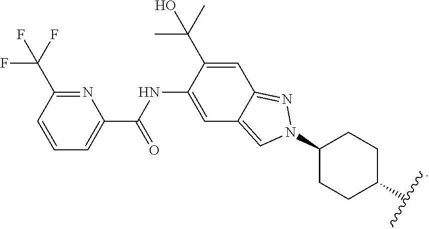 [SMILES: *[C@H]1CC[C@H](n2cc3cc(NC(=O)c4cccc(C(F)(F)F)n4)c(C(C)(C)O)cc3n2)CC1]

In some embodiments, IRAK is

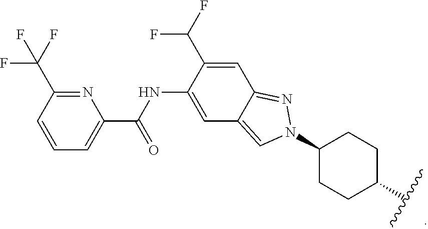 [SMILES: *[C@H]1CC[C@H](n2cc3cc(NC(=O)c4cccc(C(F)(F)F)n4)c(C(F)F)cc3n2)CC1]

In some embodiments, IRAK is

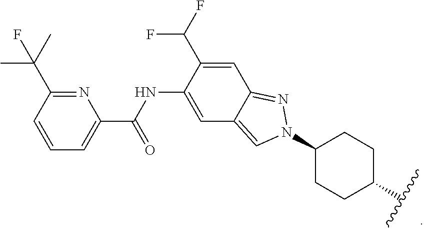 [SMILES: *[C@H]1CC[C@H](n2cc3cc(NC(=O)c4cccc(C(C)(C)F)n4)c(C(F)F)cc3n2)CC1]

In some embodiments, IRAK is

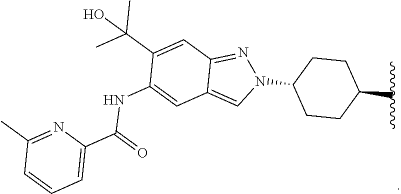 [SMILES: *[C@H]1CC[C@H](n2cc3cc(NC(=O)c4cccc(C)n4)c(C(C)(C)O)cc3n2)CC1]

In some embodiments, IRAK is

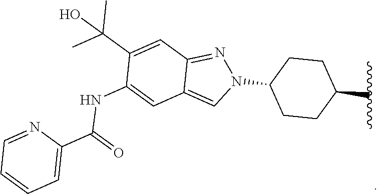 [SMILES: *[C@H]1CC[C@H](n2cc3cc(NC(=O)c4ccccn4)c(C(C)(C)O)cc3n2)CC1]

In some embodiments, IRAK is

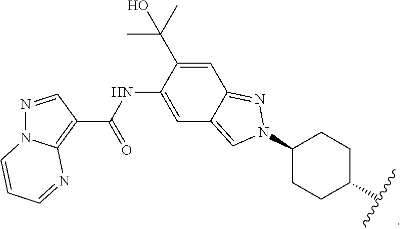 [SMILES: *[C@H]1CC[C@H](n2cc3cc(NC(=O)c4cnn5cccnc45)c(C(C)(C)O)cc3n2)CC1]

In some embodiments, IRAK is

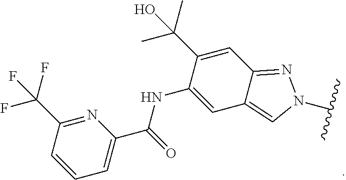 [SMILES: *n1cc2cc(NC(=O)c3cccc(C(F)(F)F)n3)c(C(C)(C)O)cc2n1]

In some embodiments, IRAK is

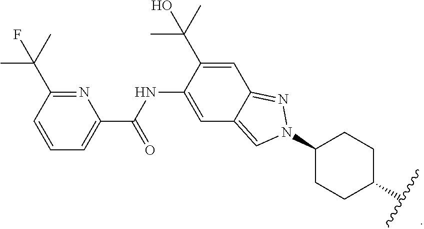 [SMILES: *[C@H]1CC[C@H](n2cc3cc(NC(=O)c4cccc(C(C)(C)F)n4)c(C(C)(C)O)cc3n2)CC1]

In some embodiments, IRAK is

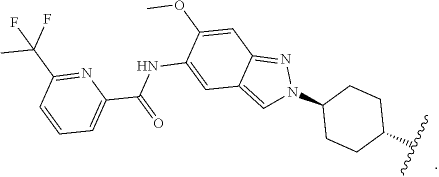 [SMILES: *[C@H]1CC[C@H](n2cc3cc(NC(=O)c4cccc(C(C)(F)F)n4)c(OC)cc3n2)CC1]

In some embodiments, IRAK is

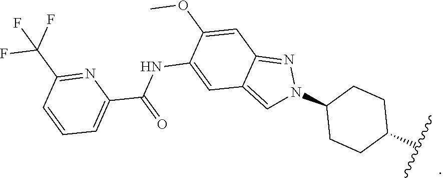 [SMILES: *[C@H]1CC[C@H](n2cc3cc(NC(=O)c4cccc(C(F)(F)F)n4)c(OC)cc3n2)CC1]

In some embodiments, IRAK is

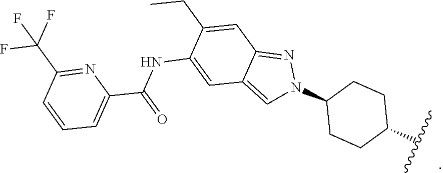 [SMILES: *[C@H]1CC[C@H](n2cc3cc(NC(=O)c4cccc(C(F)(F)F)n4)c(CC)cc3n2)CC1]

In some embodiments, IRAK is

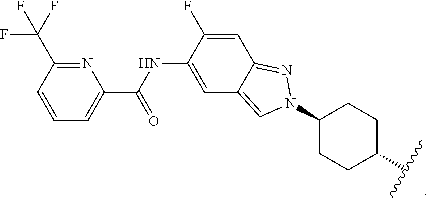 [SMILES: *[C@H]1CC[C@H](n2cc3cc(NC(=O)c4cccc(C(F)(F)F)n4)c(F)cc3n2)CC1]

In some embodiments, IRAK is

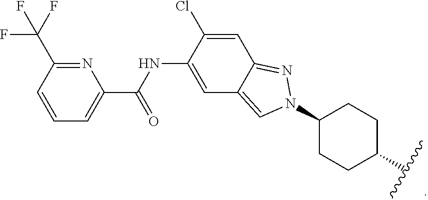 [SMILES: *[C@H]1CC[C@H](n2cc3cc(NC(=O)c4cccc(C(F)(F)F)n4)c(Cl)cc3n2)CC1]

In some embodiments, IRAK is

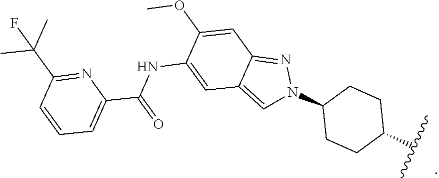 [SMILES: *[C@H]1CC[C@H](n2cc3cc(NC(=O)c4cccc(C(C)(C)F)n4)c(OC)cc3n2)CC1]

In some embodiments, IRAK is

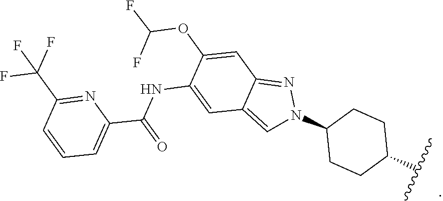 [SMILES: *[C@H]1CC[C@H](n2cc3cc(NC(=O)c4cccc(C(F)(F)F)n4)c(OC(F)F)cc3n2)CC1]

In some embodiments, IRAK is

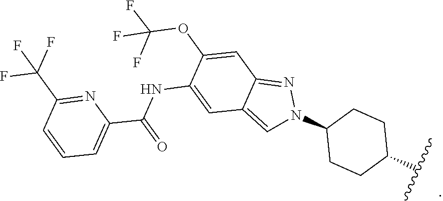 [SMILES: *[C@H]1CC[C@H](n2cc3cc(NC(=O)c4cccc(C(F)(F)F)n4)c(OC(F)(F)F)cc3n2)CC1]

In some embodiments, IRAK is

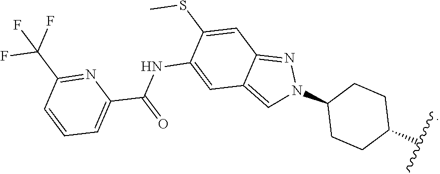 [SMILES: *[C@H]1CC[C@H](n2cc3cc(NC(=O)c4cccc(C(F)(F)F)n4)c(SC)cc3n2)CC1]

In some embodiments, IRAK is

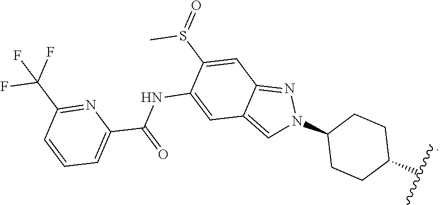 [SMILES: *[C@H]1CC[C@H](n2cc3cc(NC(=O)c4cccc(C(F)(F)F)n4)c(S(C)=O)cc3n2)CC1]

In some embodiments, IRAK is

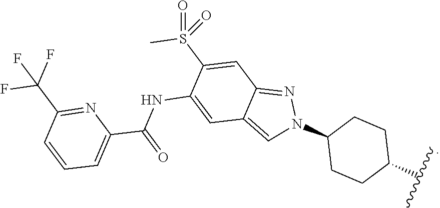 [SMILES: *[C@H]1CC[C@H](n2cc3cc(NC(=O)c4cccc(C(F)(F)F)n4)c(S(C)(=O)=O)cc3n2)CC1]

In some embodiments, IRAK is

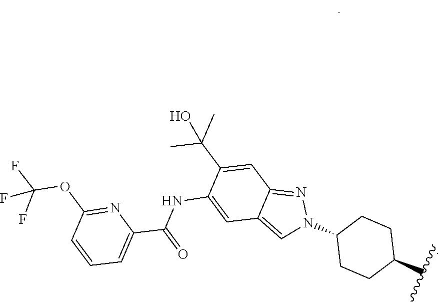 [SMILES: *[C@H]1CC[C@H](n2cc3cc(NC(=O)c4cccc(OC(F)(F)F)n4)c(C(C)(C)O)cc3n2)CC1]

In some embodiments, IRAK is

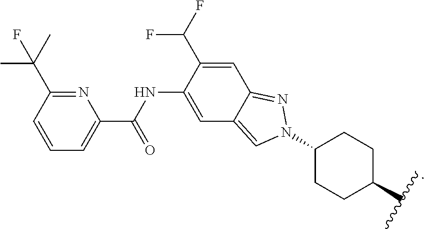 [SMILES: *[C@H]1CC[C@H](n2cc3cc(NC(=O)c4cccc(C(C)(C)F)n4)c(C(F)F)cc3n2)CC1]

In some embodiments, IRAK is

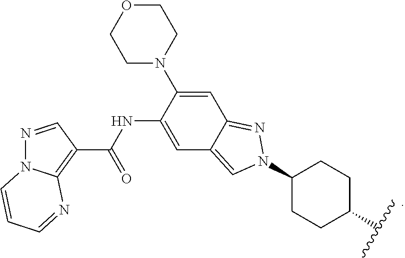 [SMILES: *[C@H]1CC[C@H](n2cc3cc(NC(=O)c4cnn5cccnc45)c(N4CCOCC4)cc3n2)CC1]

In some embodiments, IRAK is

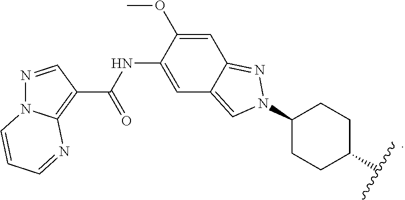 [SMILES: *[C@H]1CC[C@H](n2cc3cc(NC(=O)c4cnn5cccnc45)c(OC)cc3n2)CC1]

In some embodiments, IRAK is

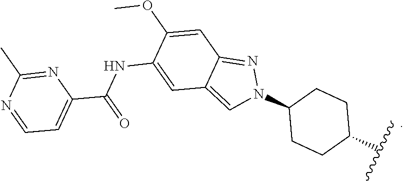 [SMILES: *[C@H]1CC[C@H](n2cc3cc(NC(=O)c4ccnc(C)n4)c(OC)cc3n2)CC1]

In some embodiments, IRAK is

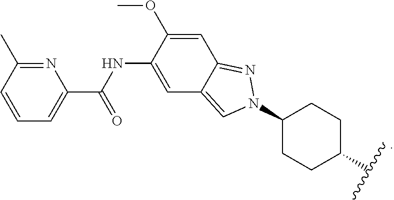 [SMILES: *[C@H]1CC[C@H](n2cc3cc(NC(=O)c4cccc(C)n4)c(OC)cc3n2)CC1]

In some embodiments, IRAK is

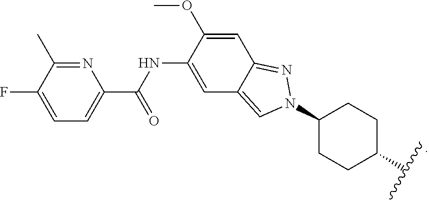 [SMILES: *[C@H]1CC[C@H](n2cc3cc(NC(=O)c4ccc(F)c(C)n4)c(OC)cc3n2)CC1]

In some embodiments, IRAK is

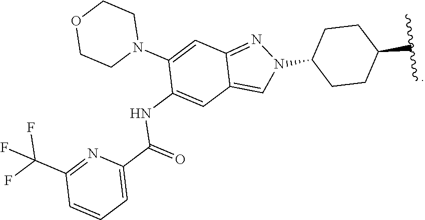 [SMILES: *[C@H]1CC[C@H](n2cc3cc(NC(=O)c4cccc(C(F)(F)F)n4)c(N4CCOCC4)cc3n2)CC1]

In some embodiments, IRAK is

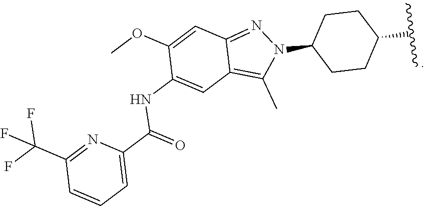 [SMILES: *[C@H]1CC[C@H](n2nc3cc(OC)c(NC(=O)c4cccc(C(F)(F)F)n4)cc3c2C)CC1]

In some embodiments, IRAK is

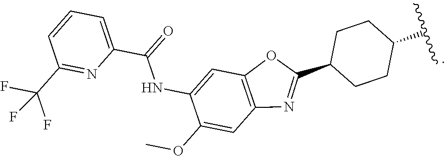 [SMILES: *[C@H]1CC[C@H](c2nc3cc(OC)c(NC(=O)c4cccc(C(F)(F)F)n4)cc3o2)CC1]

In some embodiments, IRAK is

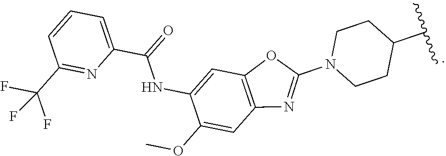 [SMILES: *C1CCN(c2nc3cc(OC)c(NC(=O)c4cccc(C(F)(F)F)n4)cc3o2)CC1]

In some embodiments, IRAK is

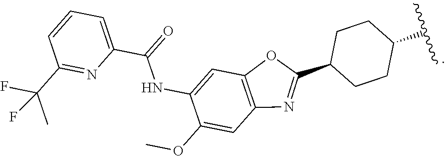 [SMILES: *[C@H]1CC[C@H](c2nc3cc(OC)c(NC(=O)c4cccc(C(C)(F)F)n4)cc3o2)CC1]

In some embodiments, IRAK is

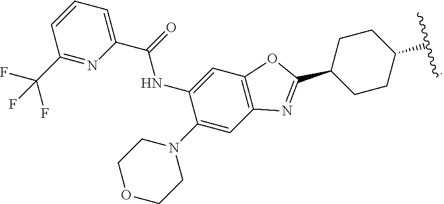 [SMILES: *[C@H]1CC[C@H](c2nc3cc(N4CCOCC4)c(NC(=O)c4cccc(C(F)(F)F)n4)cc3o2)CC1]

In some embodiments, IRAK is

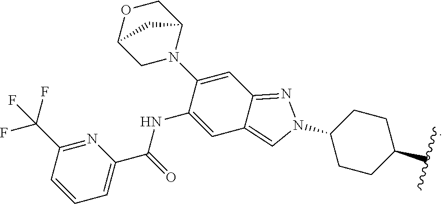

In some embodiments, IRAK is

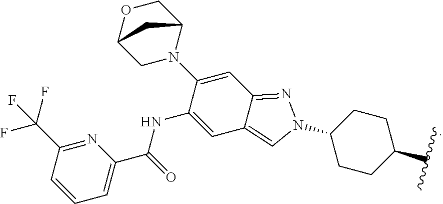 [SMILES: *[C@H]1CC[C@H](n2cc3cc(NC(=O)c4cccc(C(F)(F)F)n4)c(N4C[C@H]5C[C@@H]4CO5)cc3n2)CC1]

In some embodiments, IRAK is

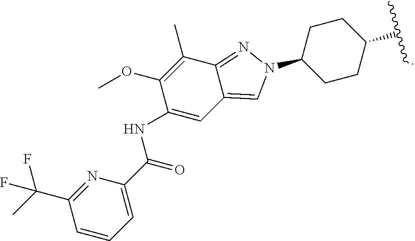 [SMILES: *[C@H]1CC[C@H](n2cc3cc(NC(=O)c4cccc(C(C)(F)F)n4)c(OC)c(C)c3n2)CC1]

In some embodiments, IRAK is

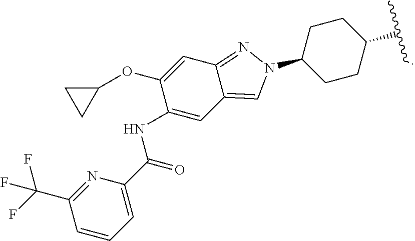 [SMILES: *[C@H]1CC[C@H](n2cc3cc(NC(=O)c4cccc(C(F)(F)F)n4)c(OC4CC4)cc3n2)CC1]

In some embodiments, IRAK is

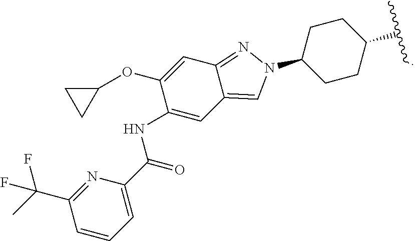 [SMILES: *[C@H]1CC[C@H](n2cc3cc(NC(=O)c4cccc(C(C)(F)F)n4)c(OC4CC4)cc3n2)CC1]

In some embodiments, IRAK is

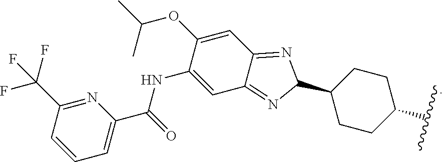 [SMILES: *[C@H]1CC[C@H](C2N=c3cc(NC(=O)c4cccc(C(F)(F)F)n4)c(OC(C)C)cc3=N2)CC1]

In some embodiments, IRAK is

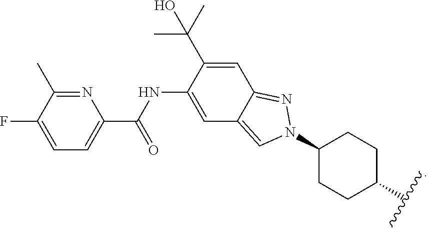 [SMILES: *[C@H]1CC[C@H](n2cc3cc(NC(=O)c4ccc(F)c(C)n4)c(C(C)(C)O)cc3n2)CC1]

In some embodiments, IRAK is

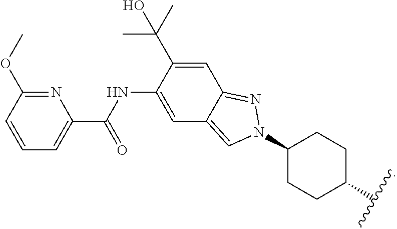 [SMILES: *[C@H]1CC[C@H](n2cc3cc(NC(=O)c4cccc(OC)n4)c(C(C)(C)O)cc3n2)CC1]

In some embodiments, IRAK is

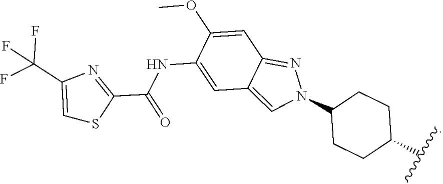

In some embodiments, IRAK is

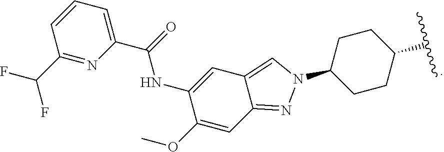 [SMILES: *[C@H]1CC[C@H](n2cc3cc(NC(=O)c4cccc(C(F)F)n4)c(OC)cc3n2)CC1]

In some embodiments, IRAK is

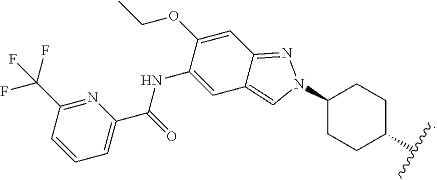 [SMILES: *[C@H]1CC[C@H](n2cc3cc(NC(=O)c4cccc(C(F)(F)F)n4)c(OCC)cc3n2)CC1]

In some embodiments, IRAK is

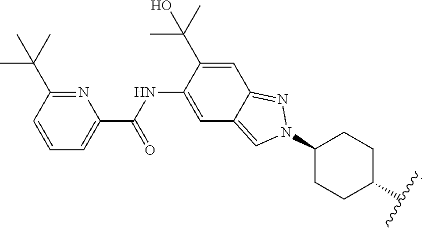 [SMILES: *[C@H]1CC[C@H](n2cc3cc(NC(=O)c4cccc(C(C)(C)C)n4)c(C(C)(C)O)cc3n2)CC1]

In some embodiments, IRAK is

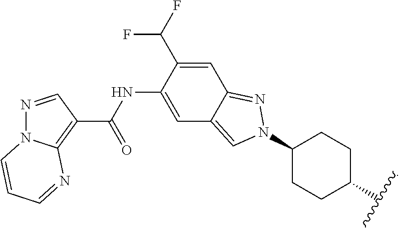 [SMILES: *[C@H]1CC[C@H](n2cc3cc(NC(=O)c4cnn5cccnc45)c(C(F)F)cc3n2)CC1]

In some embodiments, IRAK is

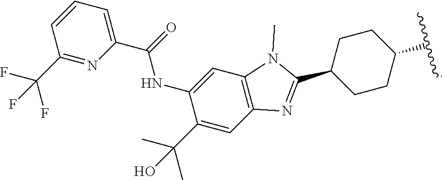 [SMILES: *[C@H]1CC[C@H](c2nc3cc(C(C)(C)O)c(NC(=O)c4cccc(C(F)(F)F)n4)cc3n2C)CC1]

In some embodiments, IRAK is

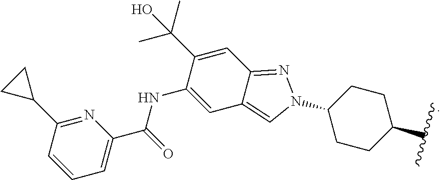 [SMILES: *[C@H]1CC[C@H](n2cc3cc(NC(=O)c4cccc(C5CC5)n4)c(C(C)(C)O)cc3n2)CC1]

In some embodiments, IRAK is

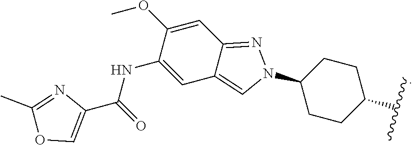 [SMILES: *[C@H]1CC[C@H](n2cc3cc(NC(=O)c4coc(C)n4)c(OC)cc3n2)CC1]

In some embodiments, IRAK is

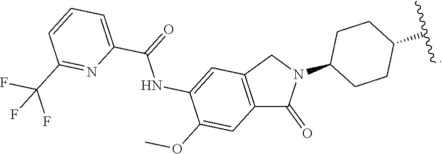 [SMILES: *[C@H]1CC[C@H](N2Cc3cc(NC(=O)c4cccc(C(F)(F)F)n4)c(OC)cc3C2=O)CC1]

In some embodiments, IRAK is

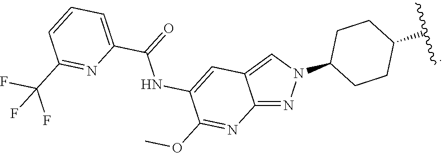 [SMILES: *[C@H]1CC[C@H](n2cc3cc(NC(=O)c4cccc(C(F)(F)F)n4)c(OC)nc3n2)CC1]

In some embodiments, IRAK is

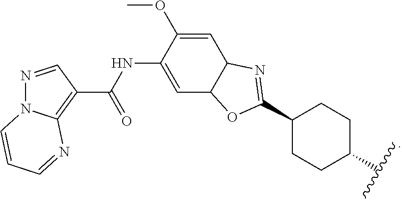 [SMILES: *[C@H]1CC[C@H](C2=NC3C=C(OC)C(NC(=O)c4cnn5cccnc45)=CC3O2)CC1]

In some embodiments, IRAK is

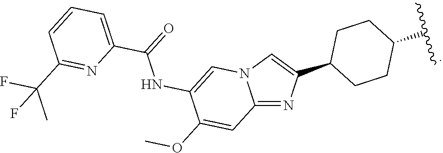 [SMILES: *[C@H]1CC[C@H](c2cn3cc(NC(=O)c4cccc(C(C)(F)F)n4)c(OC)cc3n2)CC1]

In some embodiments, IRAK is

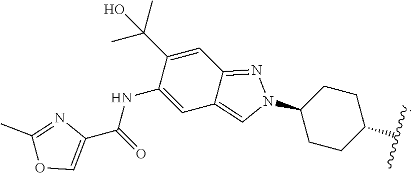 [SMILES: *[C@H]1CC[C@H](n2cc3cc(NC(=O)c4coc(C)n4)c(C(C)(C)O)cc3n2)CC1]

In some embodiments, IRAK is

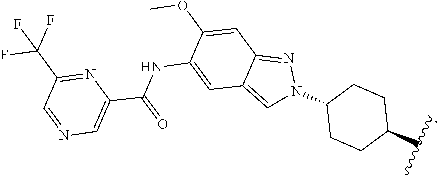 [SMILES: *[C@H]1CC[C@H](n2cc3cc(NC(=O)c4cncc(C(F)(F)F)n4)c(OC)cc3n2)CC1]

In some embodiments, IRAK is

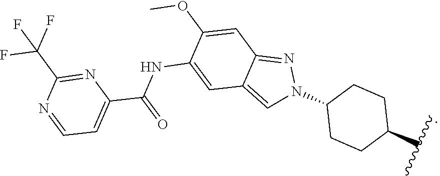 [SMILES: *[C@H]1CC[C@H](n2cc3cc(NC(=O)c4ccnc(C(F)(F)F)n4)c(OC)cc3n2)CC1]

In some embodiments, IRAK is

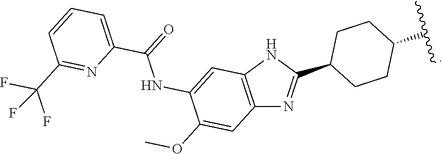 [SMILES: *[C@H]1CC[C@H](c2nc3cc(OC)c(NC(=O)c4cccc(C(F)(F)F)n4)cc3[nH]2)CC1]

In some embodiments, IRAK is

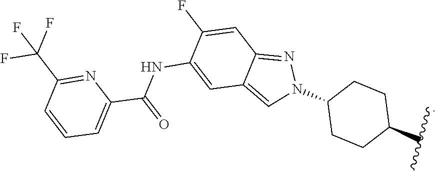 [SMILES: *[C@H]1CC[C@H](n2cc3cc(NC(=O)c4cccc(C(F)(F)F)n4)c(F)cc3n2)CC1]

In some embodiments, IRAK is

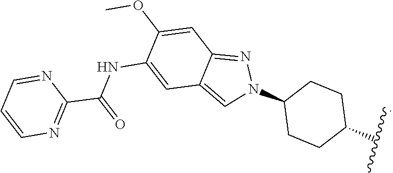 [SMILES: *[C@H]1CC[C@H](n2cc3cc(NC(=O)c4ncccn4)c(OC)cc3n2)CC1]

In some embodiments, IRAK is

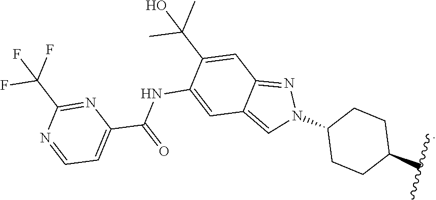 [SMILES: *[C@H]1CC[C@H](n2cc3cc(NC(=O)c4ccnc(C(F)(F)F)n4)c(C(C)(C)O)cc3n2)CC1]

In some embodiments, IRAK is

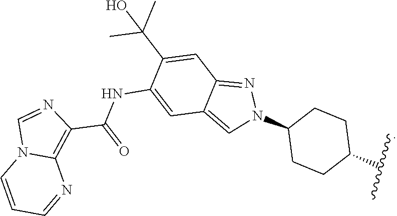 [SMILES: *[C@H]1CC[C@H](n2cc3cc(NC(=O)c4ncn5cccnc45)c(C(C)(C)O)cc3n2)CC1]

In some embodiments, IRAK is

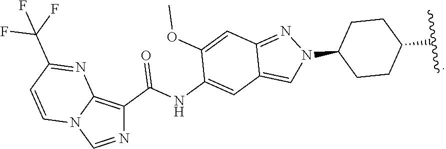 [SMILES: *[C@H]1CC[C@H](n2cc3cc(NC(=O)c4ncn5ccc(C(F)(F)F)nc45)c(OC)cc3n2)CC1]

In some embodiments, IRAK is

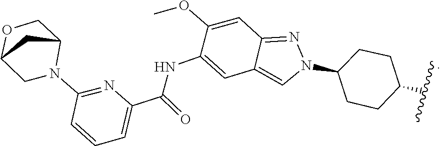 [SMILES: *[C@H]1CC[C@H](n2cc3cc(NC(=O)c4cccc(N5C[C@H]6C[C@@H]5CO6)n4)c(OC)cc3n2)CC1]

In some embodiments, IRAK is

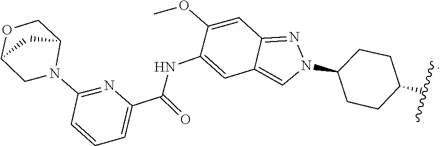 [SMILES: *[C@H]1CC[C@H](n2cc3cc(NC(=O)c4cccc(N5C[C@@H]6C[C@H]5CO6)n4)c(OC)cc3n2)CC1]

In some embodiments, IRAK is

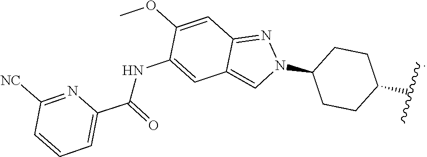 [SMILES: *[C@H]1CC[C@H](n2cc3cc(NC(=O)c4cccc(C#N)n4)c(OC)cc3n2)CC1]

In some embodiments, IRAK is

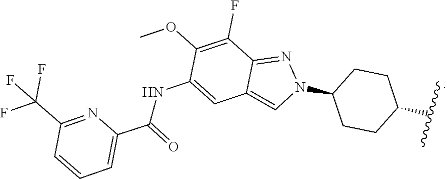 [SMILES: *[C@H]1CC[C@H](n2cc3cc(NC(=O)c4cccc(C(F)(F)F)n4)c(OC)c(F)c3n2)CC1]

In some embodiments, IRAK is

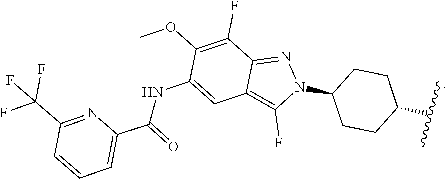 [SMILES: *[C@H]1CC[C@H](n2nc3c(F)c(OC)c(NC(=O)c4cccc(C(F)(F)F)n4)cc3c2F)CC1]

In some embodiments, IRAK is

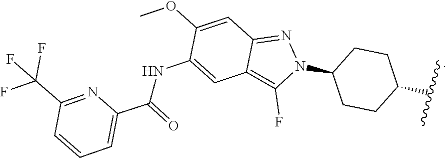 [SMILES: *[C@H]1CC[C@H](n2nc3cc(OC)c(NC(=O)c4cccc(C(F)(F)F)n4)cc3c2F)CC1]

In some embodiments, IRAK is

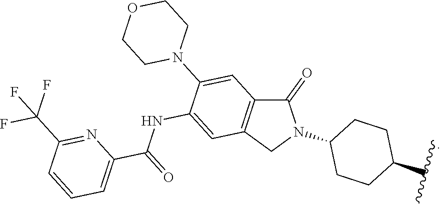 [SMILES: *[C@H]1CC[C@H](N2Cc3cc(NC(=O)c4cccc(C(F)(F)F)n4)c(N4CCOCC4)cc3C2=O)CC1]

In some embodiments, IRAK is

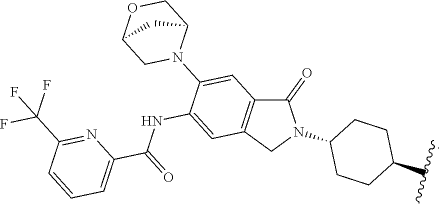 [SMILES: *[C@H]1CC[C@H](N2Cc3cc(NC(=O)c4cccc(C(F)(F)F)n4)c(N4C[C@@H]5C[C@H]4CO5)cc3C2=O)CC1]

In some embodiments, IRAK is

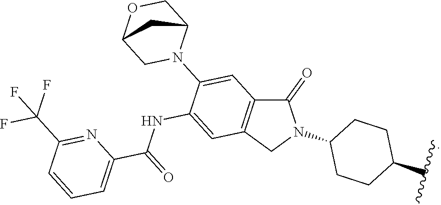 [SMILES: *[C@H]1CC[C@H](N2Cc3cc(NC(=O)c4cccc(C(F)(F)F)n4)c(N4C[C@H]5C[C@@H]4CO5)cc3C2=O)CC1]

In some embodiments, IRAK is

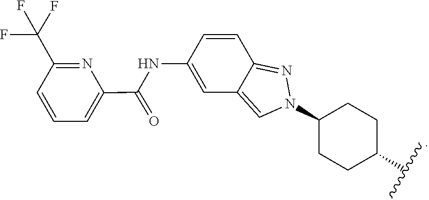 [SMILES: *[C@H]1CC[C@H](n2cc3cc(NC(=O)c4cccc(C(F)(F)F)n4)ccc3n2)CC1]

In some embodiments, IRAK is

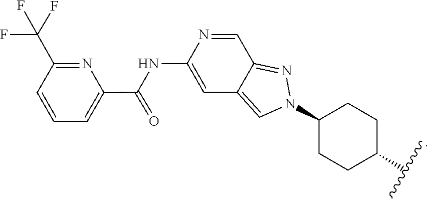 [SMILES: *[C@H]1CC[C@H](n2cc3cc(NC(=O)c4cccc(C(F)(F)F)n4)ncc3n2)CC1]

In some embodiments, IRAK is

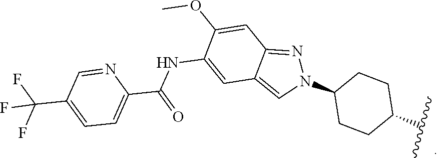 [SMILES: *[C@H]1CC[C@H](n2cc3cc(NC(=O)c4ccc(C(F)(F)F)cn4)c(OC)cc3n2)CC1]

In some embodiments, IRAK is

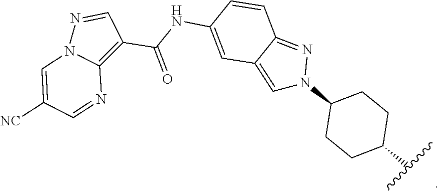 [SMILES: *[C@H]1CC[C@H](n2cc3cc(NC(=O)c4cnn5cc(C#N)cnc45)ccc3n2)CC1]

In some embodiments, IRAK is

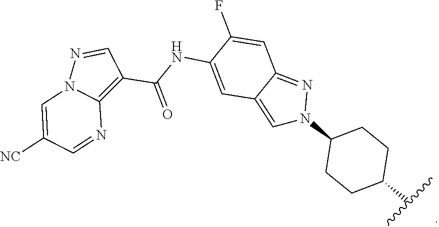 [SMILES: *[C@H]1CC[C@H](n2cc3cc(NC(=O)c4cnn5cc(C#N)cnc45)c(F)cc3n2)CC1]

In some embodiments, IRAK is

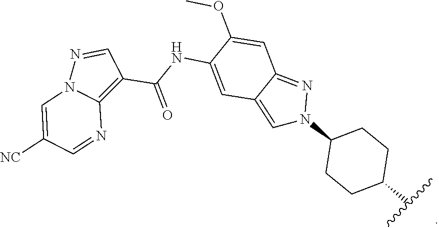 [SMILES: *[C@H]1CC[C@H](n2cc3cc(NC(=O)c4cnn5cc(C#N)cnc45)c(OC)cc3n2)CC1]

In some embodiments, IRAK is

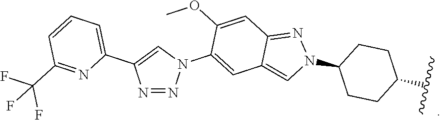 [SMILES: *[C@H]1CC[C@H](n2cc3cc(-n4cc(-c5cccc(C(F)(F)F)n5)nn4)c(OC)cc3n2)CC1]

In some embodiments, IRAK is

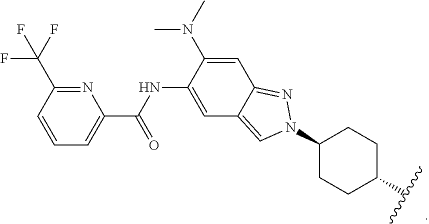 [SMILES: *[C@H]1CC[C@H](n2cc3cc(NC(=O)c4cccc(C(F)(F)F)n4)c(N(C)C)cc3n2)CC1]

In some embodiments, IRAK is

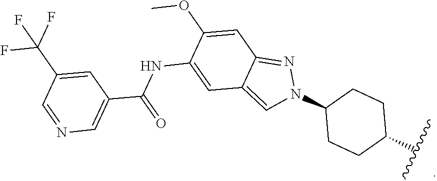 [SMILES: *[C@H]1CC[C@H](n2cc3cc(NC(=O)c4cncc(C(F)(F)F)c4)c(OC)cc3n2)CC1]

In some embodiments, IRAK is

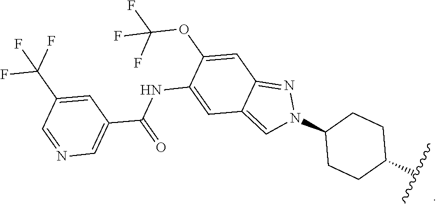 [SMILES: *[C@H]1CC[C@H](n2cc3cc(NC(=O)c4cncc(C(F)(F)F)c4)c(OC(F)(F)F)cc3n2)CC1]

In some embodiments, IRAK is

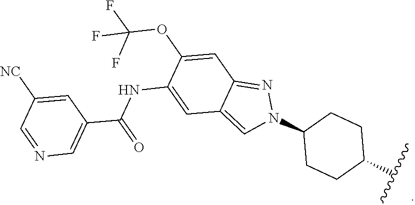 [SMILES: *[C@H]1CC[C@H](n2cc3cc(NC(=O)c4cncc(C#N)c4)c(OC(F)(F)F)cc3n2)CC1]

In some embodiments, IRAK is

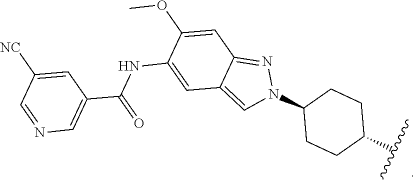 [SMILES: *[C@H]1CC[C@H](n2cc3cc(NC(=O)c4cncc(C#N)c4)c(OC)cc3n2)CC1]

In some embodiments, IRAK is

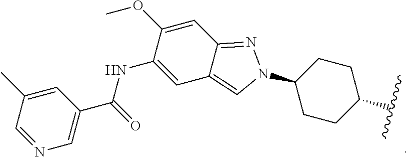 [SMILES: *[C@H]1CC[C@H](n2cc3cc(NC(=O)c4cncc(C)c4)c(OC)cc3n2)CC1]

In some embodiments, IRAK is

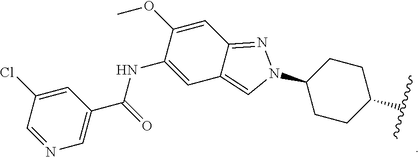 [SMILES: *[C@H]1CC[C@H](n2cc3cc(NC(=O)c4cncc(Cl)c4)c(OC)cc3n2)CC1]

In some embodiments, IRAK is

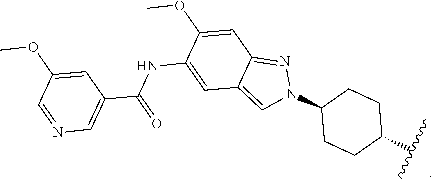 [SMILES: *[C@H]1CC[C@H](n2cc3cc(NC(=O)c4cncc(OC)c4)c(OC)cc3n2)CC1]

In some embodiments, IRAK is

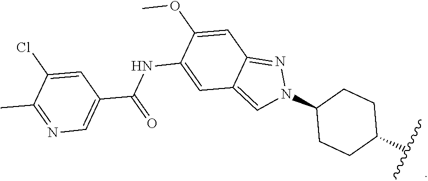 [SMILES: *[C@H]1CC[C@H](n2cc3cc(NC(=O)c4cnc(C)c(Cl)c4)c(OC)cc3n2)CC1]

In some embodiments, IRAK is

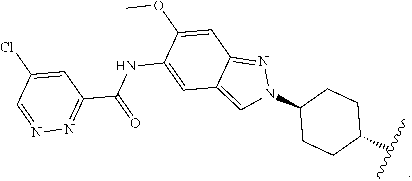 [SMILES: *[C@H]1CC[C@H](n2cc3cc(NC(=O)c4cc(Cl)cnn4)c(OC)cc3n2)CC1]

In some embodiments, IRAK is

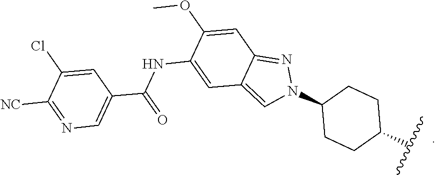 [SMILES: *[C@H]1CC[C@H](n2cc3cc(NC(=O)c4cnc(C#N)c(Cl)c4)c(OC)cc3n2)CC1]

In some embodiments, IRAK is

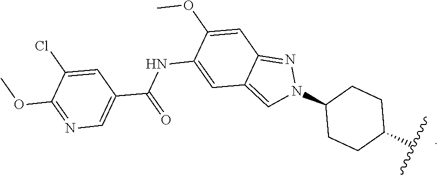 [SMILES: *[C@H]1CC[C@H](n2cc3cc(NC(=O)c4cnc(OC)c(Cl)c4)c(OC)cc3n2)CC1]

In some embodiments, IRAK is

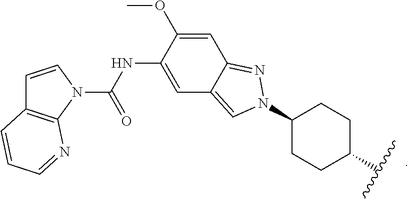 [SMILES: *[C@H]1CC[C@H](n2cc3cc(NC(=O)n4ccc5cccnc54)c(OC)cc3n2)CC1]

In some embodiments, IRAK is

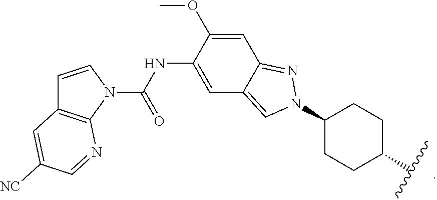 [SMILES: *[C@H]1CC[C@H](n2cc3cc(NC(=O)n4ccc5cc(C#N)cnc54)c(OC)cc3n2)CC1]

In some embodiments, IRAK is

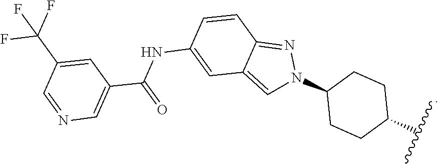 [SMILES: *[C@H]1CC[C@H](n2cc3cc(NC(=O)c4cncc(C(F)(F)F)c4)ccc3n2)CC1]

In some embodiments, IRAK is

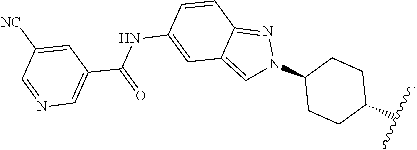 [SMILES: *[C@H]1CC[C@H](n2cc3cc(NC(=O)c4cncc(C#N)c4)ccc3n2)CC1]

In some embodiments, IRAK is

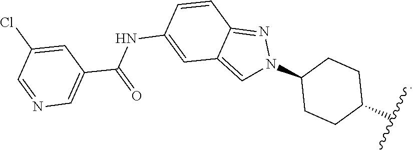 [SMILES: *[C@H]1CC[C@H](n2cc3cc(NC(=O)c4cncc(Cl)c4)ccc3n2)CC1]

In some embodiments, IRAK is

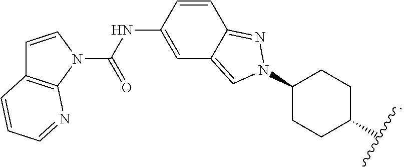 [SMILES: *[C@H]1CC[C@H](n2cc3cc(NC(=O)n4ccc5cccnc54)ccc3n2)CC1]

In some embodiments, IRAK is

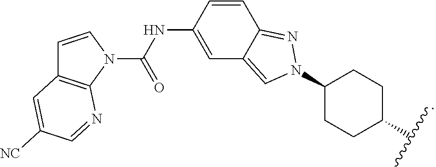 [SMILES: *[C@H]1CC[C@H](n2cc3cc(NC(=O)n4ccc5cc(C#N)cnc54)ccc3n2)CC1]

In some embodiments, IRAK is

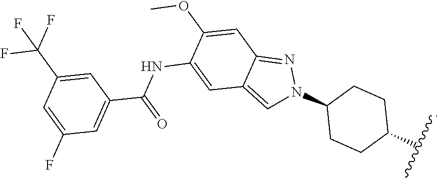 [SMILES: *[C@H]1CC[C@H](n2cc3cc(NC(=O)c4cc(F)cc(C(F)(F)F)c4)c(OC)cc3n2)CC1]

In some embodiments, IRAK is

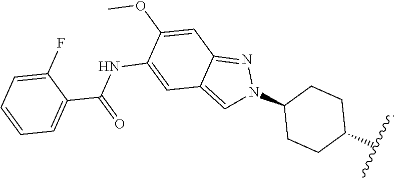 [SMILES: *[C@H]1CC[C@H](n2cc3cc(NC(=O)c4ccccc4F)c(OC)cc3n2)CC1]

In some embodiments, IRAK is

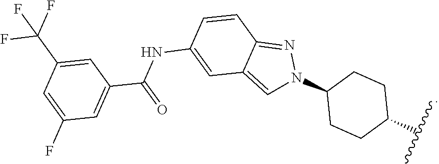 [SMILES: *[C@H]1CC[C@H](n2cc3cc(NC(=O)c4cc(F)cc(C(F)(F)F)c4)ccc3n2)CC1]

In some embodiments, IRAK is

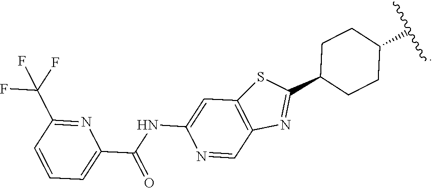 [SMILES: *[C@H]1CC[C@H](c2nc3cnc(NC(=O)c4cccc(C(F)(F)F)n4)cc3s2)CC1]

In some embodiments, IRAK is

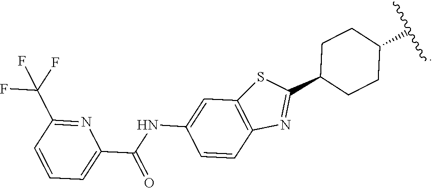 [SMILES: *[C@H]1CC[C@H](c2nc3ccc(NC(=O)c4cccc(C(F)(F)F)n4)cc3s2)CC1]

In some embodiments, IRAK is

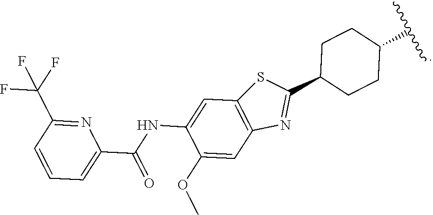 [SMILES: *[C@H]1CC[C@H](c2nc3cc(OC)c(NC(=O)c4cccc(C(F)(F)F)n4)cc3s2)CC1]

In some embodiments, IRAK is

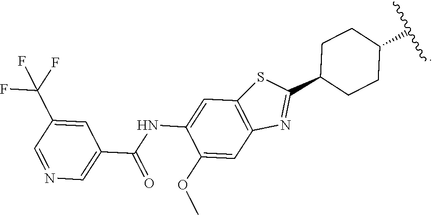 [SMILES: *[C@H]1CC[C@H](c2nc3cc(OC)c(NC(=O)c4cncc(C(F)(F)F)c4)cc3s2)CC1]

In some embodiments, IRAK is

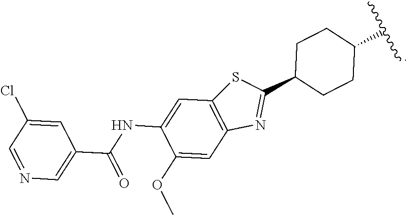 [SMILES: *[C@H]1CC[C@H](c2nc3cc(OC)c(NC(=O)c4cncc(Cl)c4)cc3s2)CC1]

In some embodiments, IRAK is

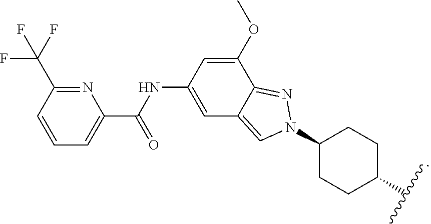 [SMILES: *[C@H]1CC[C@H](n2cc3cc(NC(=O)c4cccc(C(F)(F)F)n4)cc(OC)c3n2)CC1]

In some embodiments, IRAK is

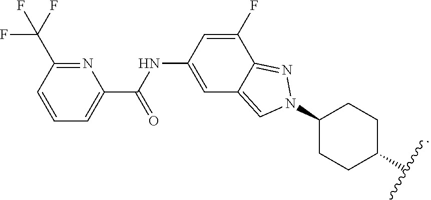 [SMILES: *[C@H]1CC[C@H](n2cc3cc(NC(=O)c4cccc(C(F)(F)F)n4)cc(F)c3n2)CC1]

In some embodiments, IRAK is

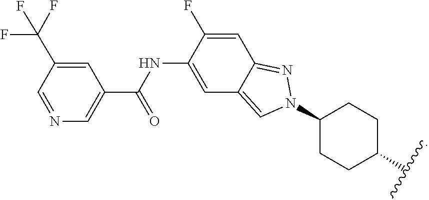 [SMILES: *[C@H]1CC[C@H](n2cc3cc(NC(=O)c4cncc(C(F)(F)F)c4)c(F)cc3n2)CC1]

In some embodiments, IRAK is

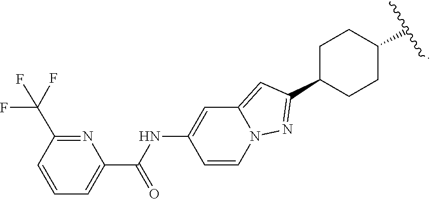 [SMILES: *[C@H]1CC[C@H](c2cc3cc(NC(=O)c4cccc(C(F)(F)F)n4)ccn3n2)CC1]

In some embodiments, IRAK is

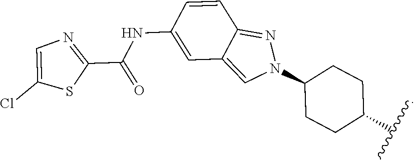 [SMILES: *[C@H]1CC[C@H](n2cc3cc(NC(=O)c4ncc(Cl)s4)ccc3n2)CC1]

In some embodiments, IRAK is

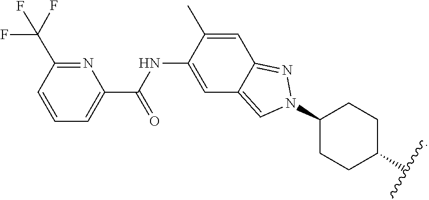 [SMILES: *[C@H]1CC[C@H](n2cc3cc(NC(=O)c4cccc(C(F)(F)F)n4)c(C)cc3n2)CC1]

In some embodiments, IRAK is

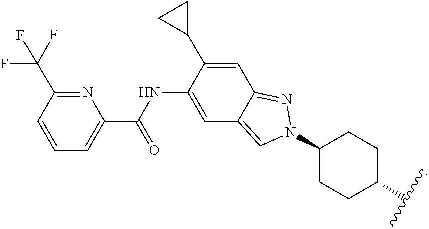 [SMILES: *[C@H]1CC[C@H](n2cc3cc(NC(=O)c4cccc(C(F)(F)F)n4)c(C4CC4)cc3n2)CC1]

In some embodiments, IRAK is

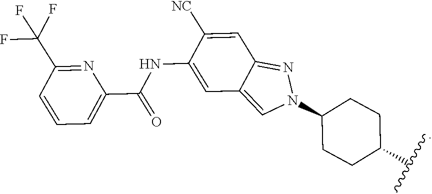 [SMILES: *[C@H]1CC[C@H](n2cc3cc(NC(=O)c4cccc(C(F)(F)F)n4)c(C#N)cc3n2)CC1]

In some embodiments, IRAK is

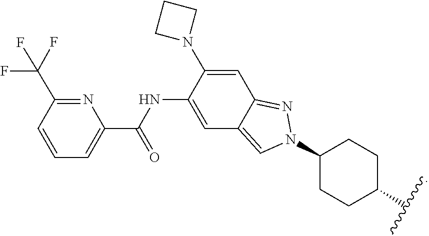 [SMILES: *[C@H]1CC[C@H](n2cc3cc(NC(=O)c4cccc(C(F)(F)F)n4)c(N4CCC4)cc3n2)CC1]

In some embodiments, IRAK is

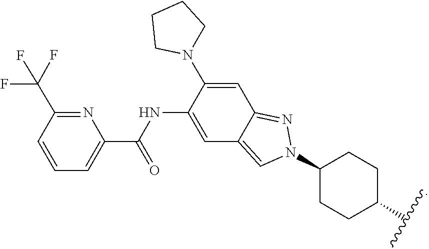 [SMILES: *[C@H]1CC[C@H](n2cc3cc(NC(=O)c4cccc(C(F)(F)F)n4)c(N4CCCC4)cc3n2)CC1]

In some embodiments, IRAK is

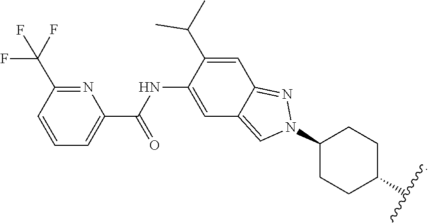 [SMILES: *[C@H]1CC[C@H](n2cc3cc(NC(=O)c4cccc(C(F)(F)F)n4)c(C(C)C)cc3n2)CC1]

In some embodiments, IRAK is

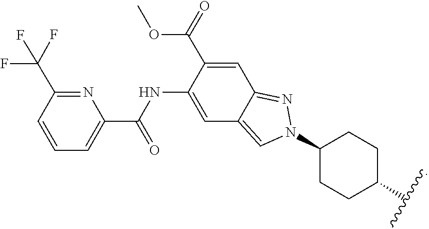 [SMILES: *[C@H]1CC[C@H](n2cc3cc(NC(=O)c4cccc(C(F)(F)F)n4)c(C(=O)OC)cc3n2)CC1]

In some embodiments, IRAK is

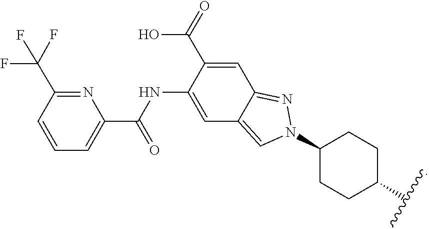 [SMILES: *[C@H]1CC[C@H](n2cc3cc(NC(=O)c4cccc(C(F)(F)F)n4)c(C(=O)O)cc3n2)CC1]

In some embodiments, IRAK is

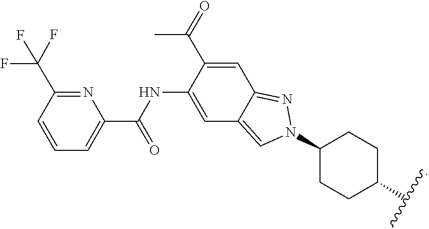 [SMILES: *[C@H]1CC[C@H](n2cc3cc(NC(=O)c4cccc(C(F)(F)F)n4)c(C(C)=O)cc3n2)CC1]

In some embodiments, IRAK is

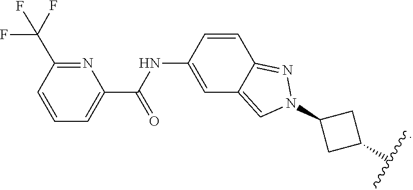 [SMILES: *[C@H]1C[C@H](n2cc3cc(NC(=O)c4cccc(C(F)(F)F)n4)ccc3n2)C1]

In some embodiments, IRAK is

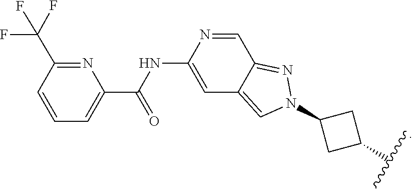 [SMILES: *[C@H]1C[C@H](n2cc3cc(NC(=O)c4cccc(C(F)(F)F)n4)ncc3n2)C1]

In some embodiments, IRAK is

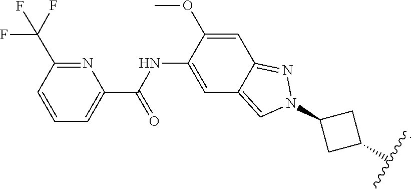 [SMILES: *[C@H]1C[C@H](n2cc3cc(NC(=O)c4cccc(C(F)(F)F)n4)c(OC)cc3n2)C1]

In some embodiments, IRAK is

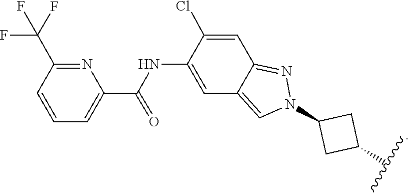 [SMILES: *[C@H]1C[C@H](n2cc3cc(NC(=O)c4cccc(C(F)(F)F)n4)c(Cl)cc3n2)C1]

In some embodiments, IRAK is

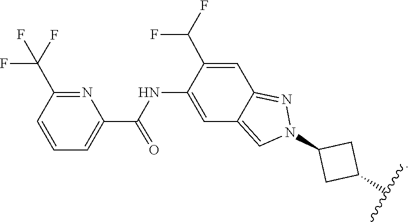 [SMILES: *[C@H]1C[C@H](n2cc3cc(NC(=O)c4cccc(C(F)(F)F)n4)c(C(F)F)cc3n2)C1]

In some embodiments, IRAK is

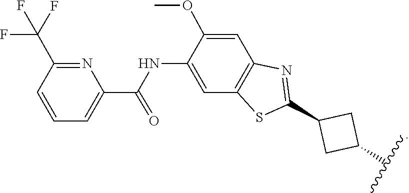 [SMILES: *[C@H]1C[C@H](c2nc3cc(OC)c(NC(=O)c4cccc(C(F)(F)F)n4)cc3s2)C1]

In some embodiments, IRAK is

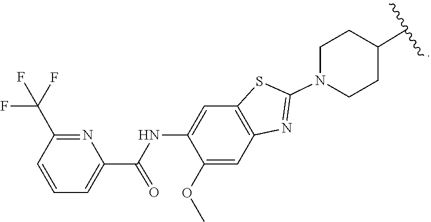 [SMILES: *C1CCN(c2nc3cc(OC)c(NC(=O)c4cccc(C(F)(F)F)n4)cc3s2)CC1]

In some embodiments, IRAK is

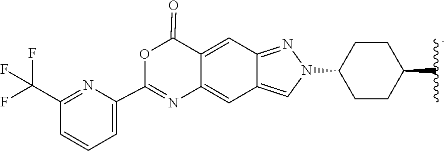 [SMILES: *[C@H]1CC[C@H](n2cc3cc4nc(-c5cccc(C(F)(F)F)n5)oc(=O)c4cc3n2)CC1]

In some embodiments, IRAK is selected from those depicted in Table 1, below.

In some embodiments, the present invention provides a compound of formula I, wherein IRAK is IRAK4 inhibitor

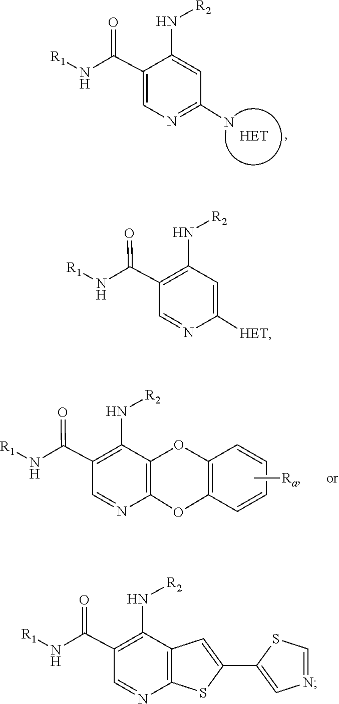

thereby forming a compound of formula I-b, I-b-1, I-b-2, or I-b-3:

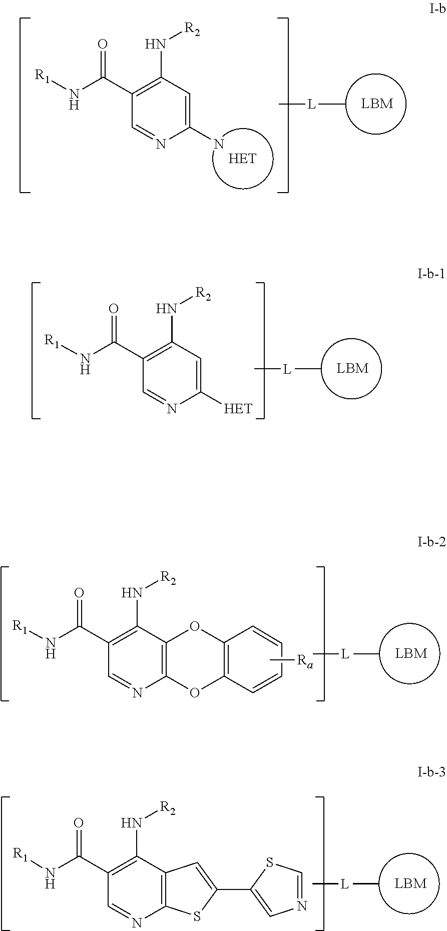

or a pharmaceutically acceptable salt thereof, wherein L and LBM are as defined above and described in embodiments herein, and wherein
HET is a heteroaryl selected from pyrazolyl, indolyl, pyrrolo[1,2-b]pyridazine, pyrrolo[2,3-b]pyridinyl, pyrrolo[2,3-d]pyrimidinyl, pyrazolo[3,4-b]pyridinyl, pyrazolo[3,4-d]pyrimidinyl, 2,3-dihydro-1H-pyrrolo[2,3-b]pyridinyl, imidazo[4,5-b]pyridinyl, and purinyl, wherein said heteroaryl is substituted with Rₐ and R_{b};
Rₐ is H, F, Cl, Br, —CN, —OH, C₁₋₄ alkyl, C₁₋₄ fluoroalkyl, C₁₋₄hydroxyalkyl, C₁₋₄ alkoxy, —NH₂, —NH(C₁₋₄alkyl), —N(C₁₋₄alkyl)₂, —NH(C₁₋₄hydroxyalkyl), —NH(C₁₋₄fluoroalkyl), —NH(C₁₋₆ hydroxy-fluoroalkyl), —C(O)NH₂, —CH₂NHC(O)(C₁₋₆ alkyl), —CH₂NHC(O)(C₁₋₆ hydroxyalkyl), —CH₂NHC(O)NH(C₁₋₆ alkyl), —CH₂NHC(O)NHCH₂(phenyl), —CH₂NHC(O)N(C₁₋₄ alkyl)₂, —CH₂NHC(O)O(C₁₋₄ alkyl), —CH₂NHC(O)(C₃₋₆ cycloalkyl), —CH₂NHC(O)(tetrahydrofuranyl), —CH₂NHC(O)CH₂(C₃₋₆ cycloalkyl), —CH₂NHC(O)CH₂(tetrahydropyranyl), —CH₂NHC(O)CH₂(phenyl), —NHC(O)(C₁₋₄ alkyl), pyrrolidinyl, hydroxypyrrolidinyl, or pyridazinyl;
R_{b} is H or —NH₂;
R¹ is:
   (i) C₁₋₆alkyl, C₁₋₆alkylC₃₋₆cycloalkyl, C₁₋₆fluoroalkyl, C₁₋₆hydroxyalkyl, C₁₋₈hydroxy-fluoroalkyl, —(C₁₋₆ alkylenyl)O(C₁₋₄ alkyl), —(C₁₋₆ alkylenyl)O(C₁₋₄fluoroalkyl), —(C₁₋₆fluoroalkylenyl)O(C₁₋₄ alkyl), —(C₁₋₆fluoroalkylenyl)O(C₁₋₄ deuteroalkyl), —(C₁₋₆ fluoroalkylenyl)O(C₁₋₄fluoroalkyl), —(C₁₋₄ fluoroalkylenyl)C(C₃₋₆ cycloalkyl)₂(OH), —(C₁₋₄alkylenyl)NHC(O)(C₁₋₄ alkylenyl)OC(O)(C₁₋₃ alkyl), —(C₁₋₆alkylenyl)NHS(O)₂(C₁₋₄ alkyl), —(C₁₋₆ alkylenyl)P(O)(C₁₋₄ alkoxy)₂, —(C₁₋₆fluoroalkylenyl)NH(C₁₋₄ alkyl), —(C₁₋₆ alkylenyl)C(O)NH(C₁₋₄ alkyl), —(C₁₋₆ fluoroalkylenyl)C(O)NH(C₁₋₄ alkyl), —(C₁₋₆ fluoroalkylenyl)C(O)NH(C₁₋₄hydroxyalkyl), or —(C₁₋₆fluoroalkylenyl)OP(O)(OH)₂;
   (ii) —(C₁₋₃ alkylenyl)Rₓ, —(C₁₋₃ fuoroalkylenyl)Rₓ, —(C₁₋₃alkylenyl)C(O)Rₓ, —(C₁₋₃ alkylenyl)C(O)NHRₓ, —(C₁₋₃fluoroalkylenyl)C(O)Rₓ, or —CH₂CF═(tetrahydropyranyl), wherein Rₓ is a cyclic group selected from C₃₋₆ cycloalkyl, tetrazolyl, 1,1-dioxidotetrahydrothiophenyl, 1,1-dioxidothiomorpholinyl, oxadiazolyl, piperidinyl, piperazinyl, pyrrolidinyl, oxetanyl, tetrahydrofuranyl, tetrahydropyranyl, pyridinyl, imidazolyl, morpholinyl, phenyl, and triazinyl, wherein each cyclic group is substituted with zero to 3 substituents independently selected from F, —OH, —CH₃, —C(CH₂)₂OH, —OCH₃, —C(O)CH₂CN, —S(O)₂CH₃, —S(O)₂NH₂, —NHC(O)CH₃, —N(S(O)₂CH₃)₂, —CH₂CH₂(acetamidophenyl), —CH₂CH₂(methoxyphenyl), —CH₂CH₂(sulfamoylphenyl), oxetanyl, benzyl, and morpholinyl;
   (iii) C₃₋₆ cycloalkyl or C₄₋₆ cycloalkenyl, each substituted with zero to 3 substituents independently selected from F, —OH, —CN, C₁₋₃alkyl, C₁₋₃ alkoxy, —S(C₁₋₃ alkyl), —NO₂, —S(O)₂(C₁₋₃ alkyl), C₁₋₄hydroxyalkyl, —C(C₁₋₃ alkyl)(OH)(C₃₋₆ cycloalkyl), —CH₂C(O)NH(C₁₋₃ alkyl), —NHC(O)(C₁₋₃ alkyl), —NHC(O)(C₁₋₄hydroxyalkyl), —C(O)NH(C₁₋₃ alkyl), —C(O)NH(C₁₋₃ deuteroalkyl), —C(O)NH(C₃₋₆ cycloalkyl), —NHC(O)O(C₁₋₃ alkyl), —NHS(O)₂(C₁₋₃alkyl), pyridinyl, imidazolyl, pyrazolyl, methylimidazolyl, methylpyrazolyl, and thiazolyl;
   (iv) tetrahydropyranyl, piperidinyl, pyrazolyl, phenyl, pyridinyl, or pyrimidinyl, each substituted with zero to 1 substituent selected from —OH, C₁₋₃ alkyl, C₁₋₃ fluoroalkyl, C₁₋₄ hydroxyalkyl, C₁₋₃alkoxy, —C(O)(C₁₋₄alkyl), —S(O)₂(C₁₋₄alkyl), —S(O)₂NH(C₁₋₄ alkyl), —NH(C₁₋₃ alkyl), —N(C₁₋₃ alkyl)₂, —O(C₁₋₃ alkylenyl)N(C₁₋₃ alkyl)₂, —CH₂(morpholinyl), azetidinyl, oxetanyl, tetrahydropyranyl, morpholinyl, piperazinyl, piperidinyl, methylpiperazinyl, methoxypiperidinyl, pyridinyl, pyrimidinyl, methylsulfonyl azetidinyl, and —C(O)(methylsulfonyl azetidinyl); or
   (v) pyrrolo[2,3-c]pyridinyl, bicyclo[2.2.1]heptan-1-ol, tetrahydrobenzo[d]thiazol-2-amine, or 1,3-diazaspiro[4.5]decane-2,4-dione; and
R₂ is:
   (i) C₁₋₇alkyl or C₂₋₆ alkenyl, each substituted with zero to three substituents independently selected from F, —OH, and —CN; —(C₁₋₄ alkylenyl)O(C₁₋₄ alkyl), —(C₁₋₄ alkylenyl)O(C₁₋₄ fluoroalkyl), —(C₁₋₆ alkylenyl)NH₂, —(C₁₋₆ alkylenyl)S(O)₂(C₁₋₃ alkyl), —(C₁₋₆ fluoroalkylenyl)NH(C₁₋₃ alkyl), or —(C₁₋₆ alkylenyl)NHC(O)(C₁₋₄fluoroalkyl);
   (ii) —(C₁₋₄ alkylenyl)R_{y} wherein R_{y} is C₃₋₆ cycloalkyl, azetidinyl, oxetanyl, oxazolyl, pyridinyl, tetrahydropyranyl, or morpholinyl, each substituted with zero to 2 substituents independently selected from F, —OH, and C₁₋₃ alkyl;
   (iii) C₃₋₆ cycloalkyl, azetidinyl, oxetanyl, furanyl, tetrahydrofuranyl, pyrrolidinyl, piperidinyl, or tetrahydropyranyl, each substituted with zero to 3 substituents independently selected from F, —OH, C₁₋₃alkyl, C₁₋₃ hydroxyalkyl, —C(O)(C₁₋₃ alkyl), —C(O)(C₁₋₃ fluoroalkyl), —C(O)(C₁₋₃ cyanoalkyl), —C(O)O(C₁₋₃ alkyl), —C(O)NH₂, —C(O)NH(C₁₋₃ alkyl), —C(O)(difluorophenyl), —NH₂, —NH(C₁₋₃ alkyl), —NH(C₁₋₃ fluoroalkyl), —NH(oxetanyl), —NHC(O)(C₁₋₃ alkyl), —NHC(O)(C₁₋₃ fluoroalkyl), —NHC(O)(C₃₋₆ cycloalkyl), —NHC(O)(fluorophenyl), —S(O)₂(C₁₋₃alkyl), imidazolyl, phenyl, pyrimidinyl, fluoropyrimidinyl, chloropyrimidinyl, and methoxypyrimidinyl;
   (iv) adamantanyl, hydroxyadamantanyl, benzo[d]imidazolyl, benzo[d]oxazolyl, benzo[d]triazolyl, benzothiazolyl, bicyclo[1.1.1]pentanyl, or hydroxy-bicyclo[2.2.1]heptanyl; or
   (v) phenyl, pyrazolyl, thiazolyl, thiadiazolyl, or indazolyl, each substituted with 0 to 2 substituents independently selected from F, Cl, —OH, —CN, C₁₋₄ alkyl, C₁₋₄ hydroxyalkyl, C₁₋₄ fluoroalkyl, C₁₋₄cyanoalkyl, C₁₋₃ alkoxy, C₃₋₆ cycloalkyl, —(C₁₋₃ alkylenyl)O(C₁₋₃alkyl), —(C₁₋₃ alkylenyl)O(C₁₋₃ fluoroalkyl), —C(O)NH₂, —C(O)NH(C₁₋₃ alkyl), —NHC(O)(C₁₋₃ alkyl), —NHC(O)S(O)₂(C₁₋₃alkyl), —S(O)₂NH₂, —S(O)₂(C₁₋₃ alkyl), pyrazolyl, methyl pyrazolyl, imidazolyl, triazolyl, methyl tetrazolyl, ethyl tetrazolyl, phenyl, pyrimidinyl, fluoropyrimidinyl, and tetrahydropyranyl;
      as defined and described in WO 2015/103453 and US 2015/0191464, the entirety of each of which is herein incorporated by reference.

In certain embodiments, the present invention provides a compound of formula I, wherein IRAK is an IRAK4 inhibitor, N HET, N O or

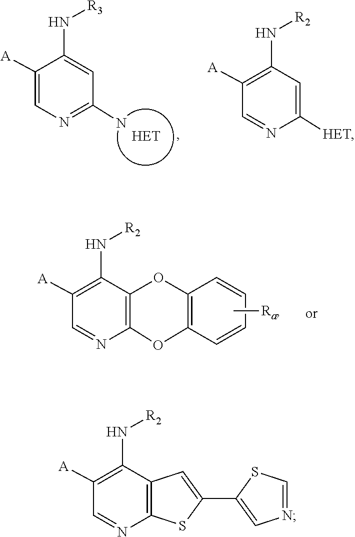

thereby forming a compound of formula I-c, I-c-1, I-c-2, or I-c-3:

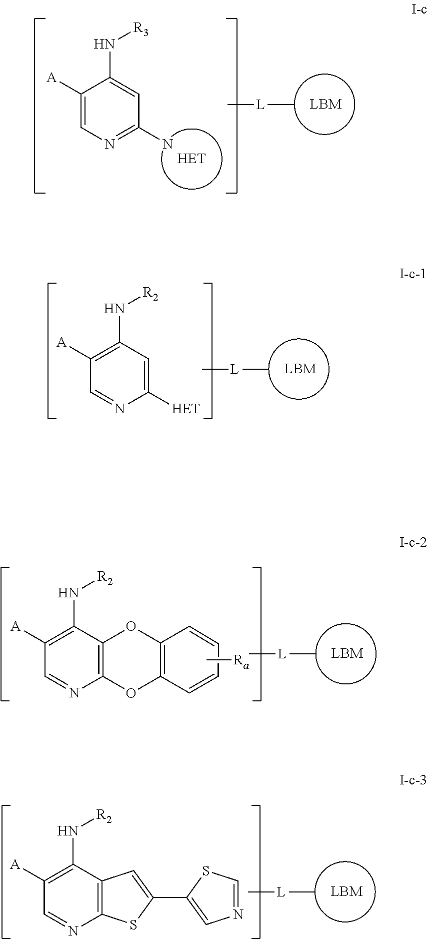

or a pharmaceutically acceptable salt thereof, wherein L and LBM are as defined above and described in embodiments herein, and wherein:
HET is a heteroaryl selected from pyrrolo[2,3-b]pyridinyl, pyrrolo[2,3-d]pyrimidinyl, pyrazolo[3,4-b]pyridinyl, pyrazolo[3,4-d]pyrimidinyl, imidazolo[4,5-b]pyridinyl, and imidazolo[4,5-d]pyrimidinyl, wherein said heteroaryl is attached to the pyridinyl group in the compound of Formula (I) by a nitrogen ring atom in said heteroaryl and wherein said heteroaryl is substituted with zero to 2 R_{b};
A is pyrazolyl, imidazolyl, triazolyl, isoxazolyl, oxadiazolyl or dihydroisoxazolyl, each substituted with Rₐ;
R₃ is C₂₋₃ alkyl, C₂₋₃ fluoroalkyl, C₃₋₄ hydroxyalkyl, or a cyclic group selected from C₃₋₆ cycloalkyl, oxetanyl, tetrahydrofuranyl, tetrahydropyranyl, and pyrazolyl, wherein said cyclic group is substituted with zero to 2 substituents independently selected from F, —OH, C₁₋₂alkyl, and —CH₂CHF₂;
Rₐ is:
(i) H, F, C₁, —OH, —CN, C₁₋₆ alkyl, C₁₋₆ fluoroalkyl, C₁₋₄ cyanoalkyl, C₁₋₆ hydroxyalkyl, C₁₋₅ hydroxy-fluoroalkyl, C₂₋₄alkenyl, C₁₋₆aminoalkyl, —(CH₂)₁₋₃NHR_{y}, —(CH₂)₁₋₃NR_{y}R_{y}, —CH₂CH(OH)(phenyl), —CH(CH₂OH)(phenyl), —CH₂CH(OH)CH₂(phenyl), —CH₂CH(OH)CH₂O(methoxyphenyl), —CH₂CH(NH₂)CH₂(phenyl), —(CH₂CH₂O)₄H, —(CH₂)₁₋₃O(C₁₋₃ alkyl), —CH₂CH(OH)CH₂O(C₁₋₃alkyl), —CH₂C(O)(C₁₋₃ alkyl), —CH₂C(O)NR_{y}R_{y}, —(CH₂)₁₋₃NR_{y}C(O)(C₁₋₃ alkyl), —CH₂C(O)O(C₁₋₃ alkyl), —C(O)NH₂, —CH₂NR_{y}C(O)NH₂, —(CH₂)₁₋₂NR_{y}C(O)O(C₁₋₂ alkyl), —(CR_{y}R_{y})₁₋₅OC(O)CH₂NR_{y}R_{y}, —CH₂CH₂S(O)₂CH₃, —CH₂S(O)₂(C₁₋₃ alkyl), —CH₂S(O)₂(phenyl), or —NH(aminocyclohexyl); or
(ii) —(CH₂)₀₋₃R_{z} or —(CH₂)₀₋₁C(O)R_{z}, wherein R_{z} is C₃₋₆cycloalkyl, azetidinyl, oxetanyl, tetrahydrofuranyl, tetrahydropyranyl, piperidinyl, piperazinyl, pyrrolyl, pyrrolidinonyl, morpholinyl, pyrrolidinyl, phenyl, pyrazolyl, imidazolyl, pyridinyl, pyrimidinyl, dioxopyrimidinyl, benzo[d]imidazolyl, benzo[d]thiazolyl, 1,3-dioxolanyl, or 8-azabicyclo[3.2.1]octanyl, each substituted with zero to 4 substituents independently from F, —CN, —OH, —NR_{y}R_{y}, C₁₋₃ alkyl, C₁₋₃ fluoroalkyl, C₁₋₃hydroxyalkyl, —CH(phenyl)₂, —O(C₁₋₄alkyl), —C(O)(C₁₋₄alkyl), —C(O)(C₁₋₄ deuteroalkyl), —C(O)(C₁₋₅ hydroxyalkyl), —C(O)(C₁₋₃ fluoroalkyl), —C(O)(C₃₋₆cycloalkyl), —C(O)O(C₁₋₃ alkyl), —C(O)NR_{y}R_{y}, —C(O)(phenyl), —C(O)(pyridinyl), —C(O)CH₂(C₃₋₆ cycloalkyl), —C(O)O(C₁₋₄ alkyl), —NH(C₁₋₄ alkyl), —NH(C₁₋₃ fluoroalkyl), —NHC(O)CH₃, —NHC(O)O(C₁₋₃alkyl), —NHC(O)OC(CH₃)₃, —S(O)₂(C₁₋₃alkyl), —OS(O)₂(C₁₋₃ alkyl), methyl oxadiazolyl, and pyrimidinyl;
each R_{b} is independently selected from H, Cl, —CN, —NH₂, and —C(O)NH₂, wherein said heteroaryl is attached to the pyridinyl group by a nitrogen atom in said heteroaryl; and
each R_{y} is independently H or C₁₋₂ alkyl;
as defined and described in WO 2016/210034 and US 2018/0186799, the entirety of each of which is herein incorporated by reference.

In some embodiments, IRAK is

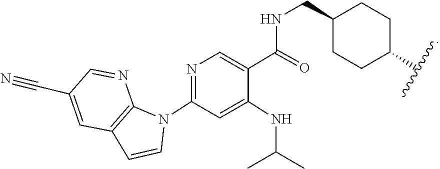 [SMILES: *[C@H]1CC[C@H](CNC(=O)c2cnc(-n3ccc4cc(C#N)cnc43)cc2NC(C)C)CC1]

In some embodiments, IRAK is

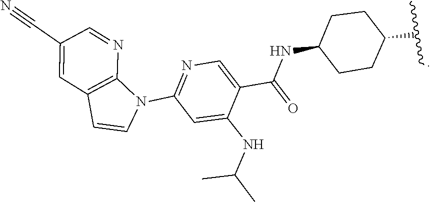 [SMILES: *[C@H]1CC[C@H](NC(=O)c2cnc(-n3ccc4cc(C#N)cnc43)cc2NC(C)C)CC1]

In some embodiments, IRAK is

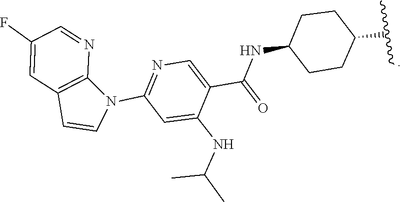 [SMILES: *[C@H]1CC[C@H](NC(=O)c2cnc(-n3ccc4cc(F)cnc43)cc2NC(C)C)CC1]

In some embodiments, IRAK is

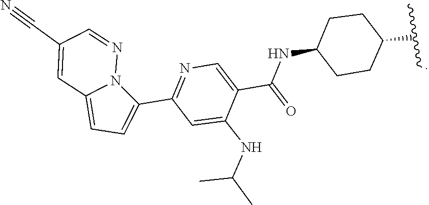 [SMILES: *[C@H]1CC[C@H](NC(=O)c2cnc(-c3ccc4cc(C#N)cnn34)cc2NC(C)C)CC1]

In some embodiments, IRAK is

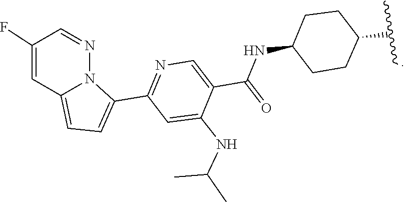 [SMILES: *[C@H]1CC[C@H](NC(=O)c2cnc(-c3ccc4cc(F)cnn34)cc2NC(C)C)CC1]

In some embodiments, IRAK is

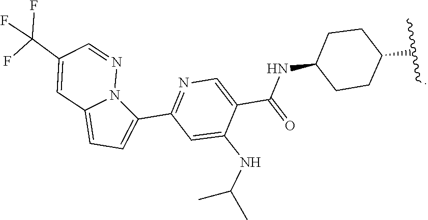 [SMILES: *[C@H]1CC[C@H](NC(=O)c2cnc(-c3ccc4cc(C(F)(F)F)cnn34)cc2NC(C)C)CC1]

In some embodiments, IRAK is

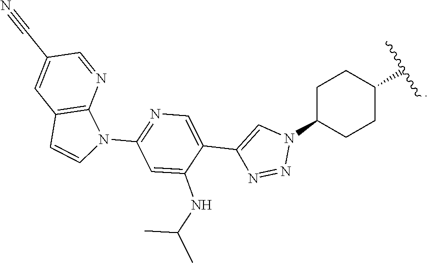 [SMILES: *[C@H]1CC[C@H](n2cc(-c3cnc(-n4ccc5cc(C#N)cnc54)cc3NC(C)C)nn2)CC1]

In some embodiments, IRAK is

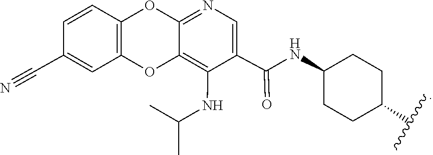 [SMILES: *[C@H]1CC[C@H](NC(=O)c2cnc3c(c2NC(C)C)Oc2cc(C#N)ccc2O3)CC1]

In some embodiments, IRAK is

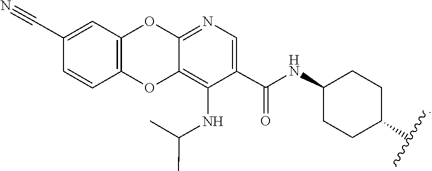 [SMILES: *[C@H]1CC[C@H](NC(=O)c2cnc3c(c2NC(C)C)Oc2ccc(C#N)cc2O3)CC1]

In some embodiments, IRAK is

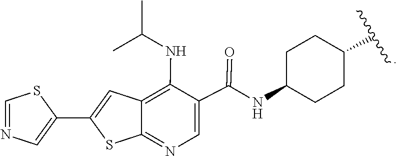 [SMILES: *[C@H]1CC[C@H](NC(=O)c2cnc3sc(-c4cncs4)cc3c2NC(C)C)CC1]

In some embodiments, IRAK is

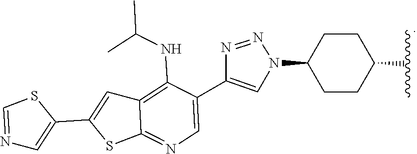

### Ligase Binding Moiety (LBM)

As defined herein and described below, wherein a formula is depicted using square brackets, L DIM L LBM e.g,

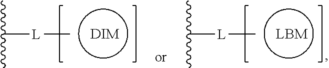

L is attached to a modifiable carbon, oxygen, or nitrogen atom within DIM or LBM including substitution or replacement of a defined group in DIM or LBM.

In some embodiments, DIM is LBM. In certain embodiments, the present invention provides a compound of formula I, wherein LBM is an E3 ubiquitin ligase (cereblon) binding moiety thereby forming a compound of formula I-aa:

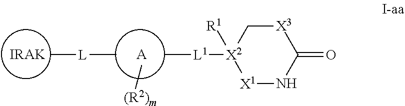

or a pharmaceutically acceptable salt thereof, wherein L and IRAK are as defined above and described herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

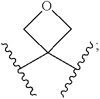 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

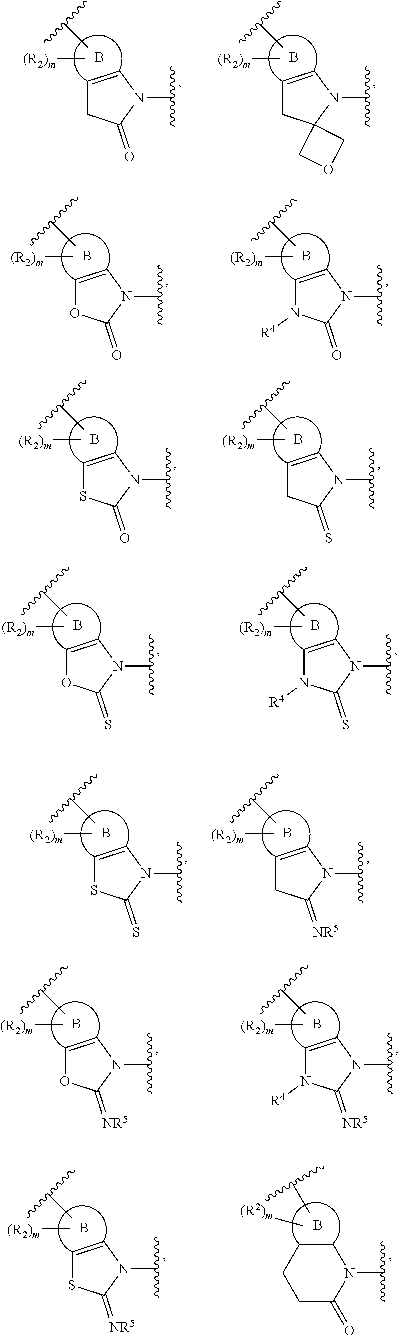

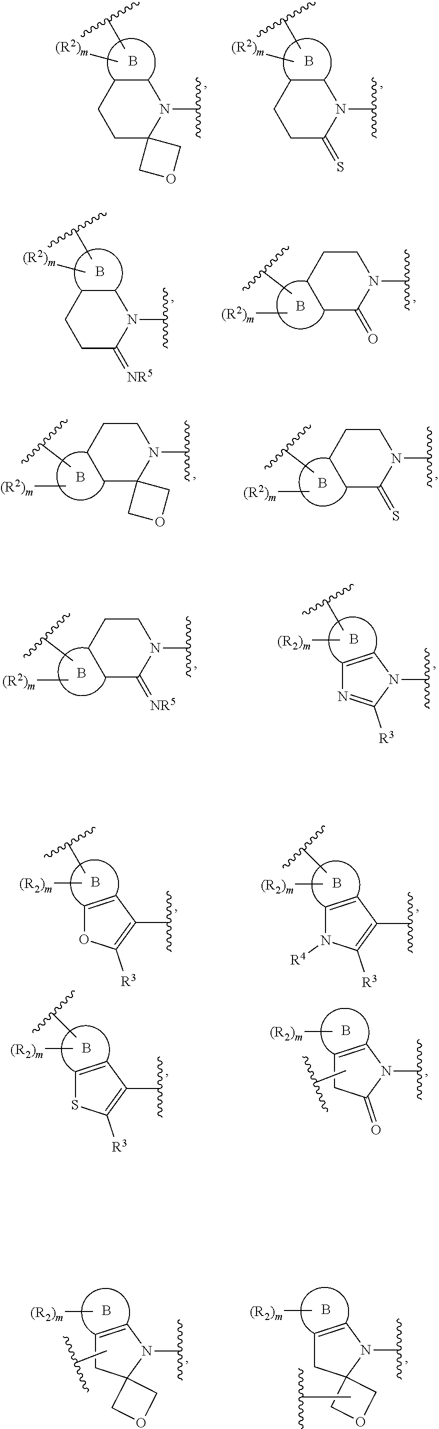

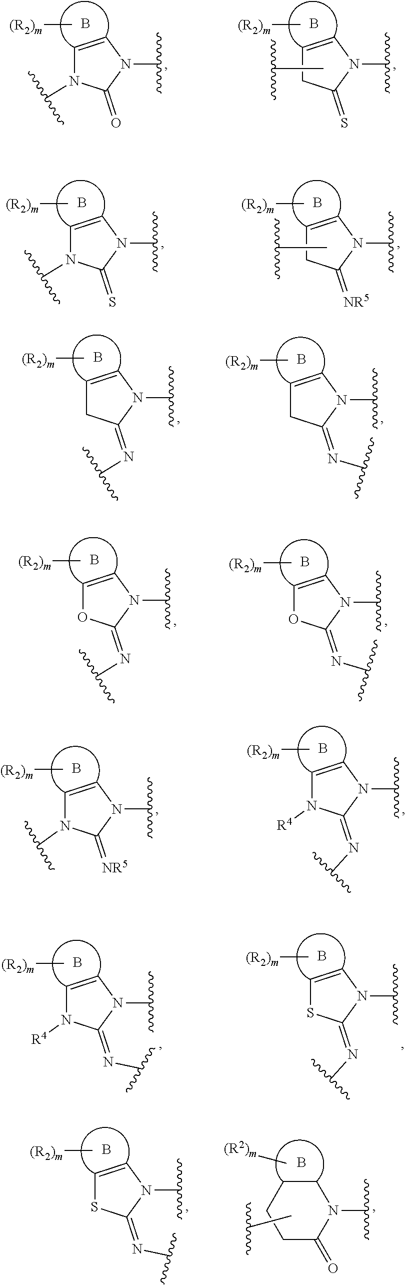

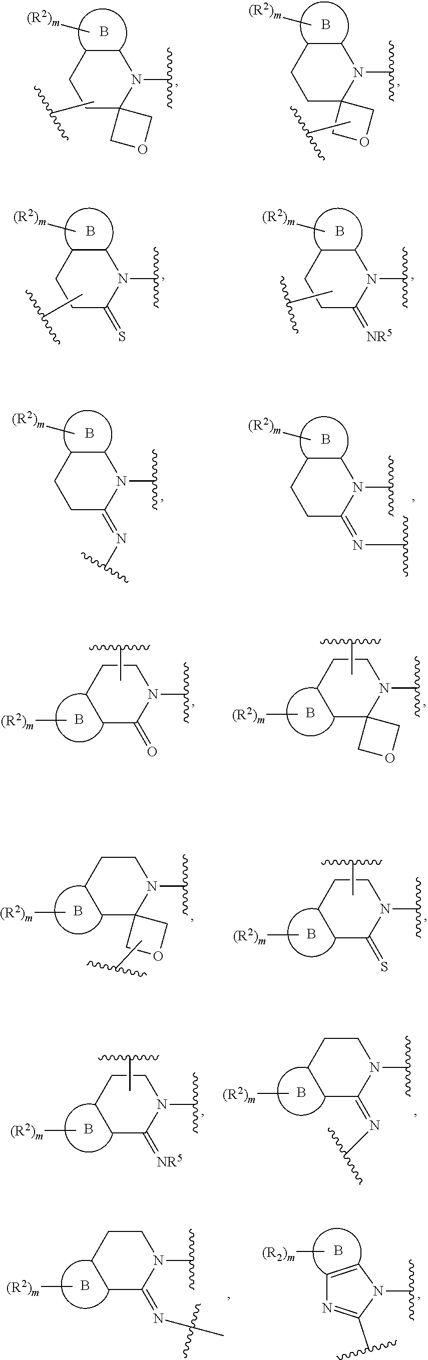

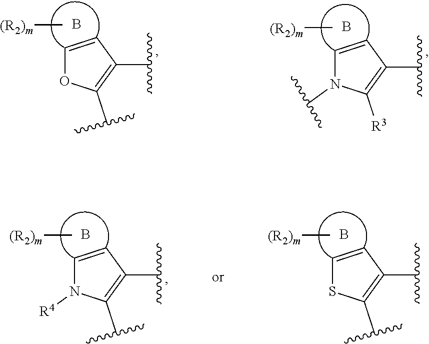

wherein
Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R group.

In some embodiments, a compound of formula I-aa above is provided as a compound of formula I-aa′ or formula I-aa″:

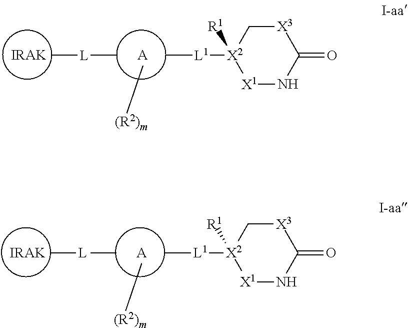

or a pharmaceutically acceptable salt thereof, wherein:
each of IRAK, Ring A, L, L¹, R¹, R², X¹, X², X³, and m is as defined above.

In certain embodiments, the present invention provides a compound of Formula I, wherein LBM is an E3 ubiquitin ligase (cereblon) binding moiety thereby forming a compound of formula I-cc:

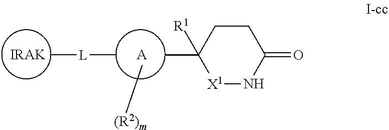

or a pharmaceutically acceptable salt thereof, wherein L and IRAK are as defined above and described in embodiments herein, and wherein:
X¹ is a bivalent moiety selected from a covalent bond, —CH₂—, —C(O)—, —C(S)—, or

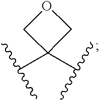 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring A is a bi- or tricyclic ring selected from

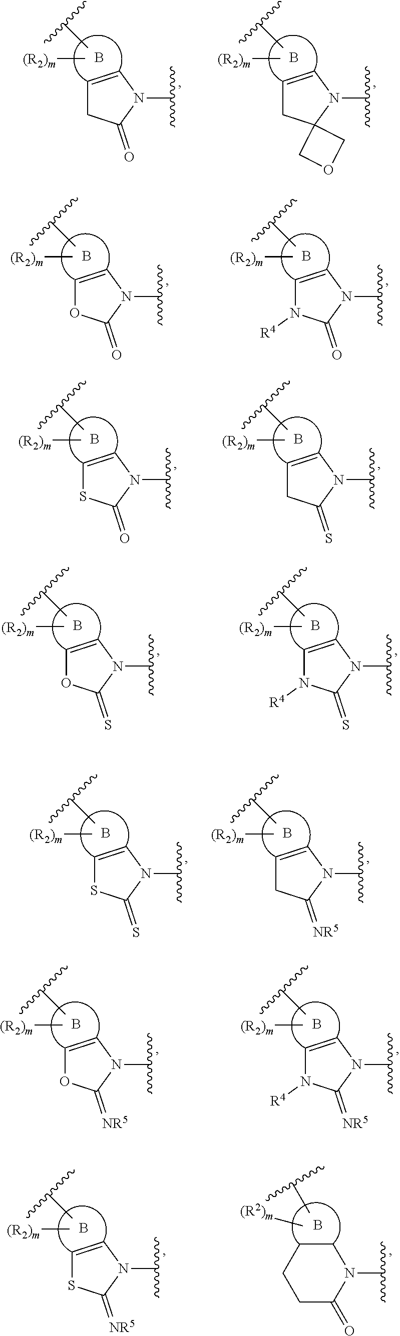

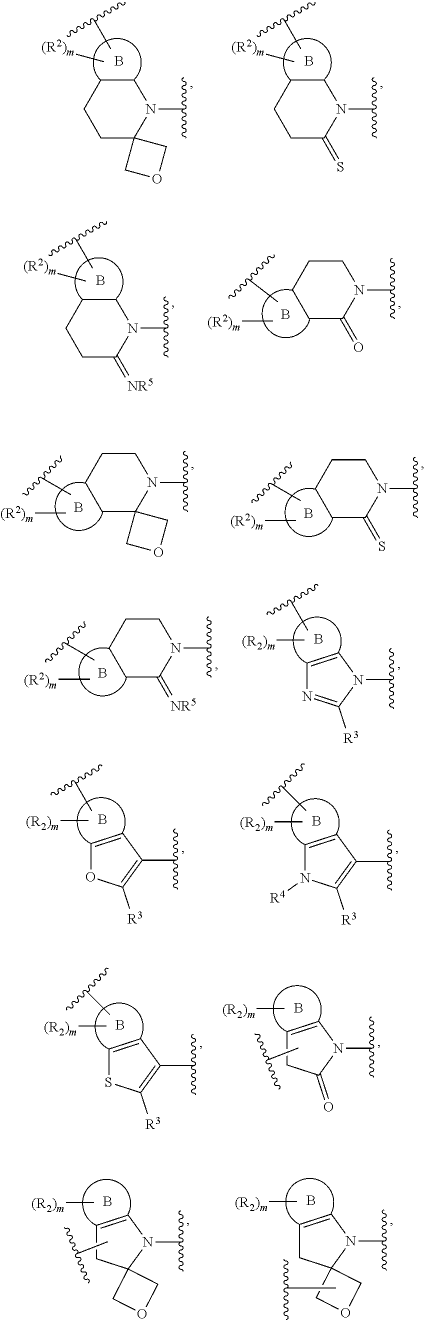

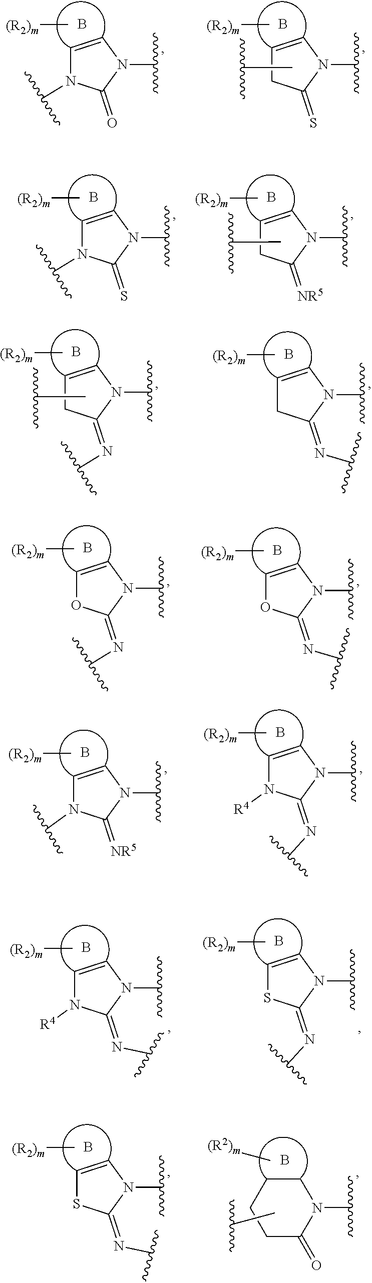

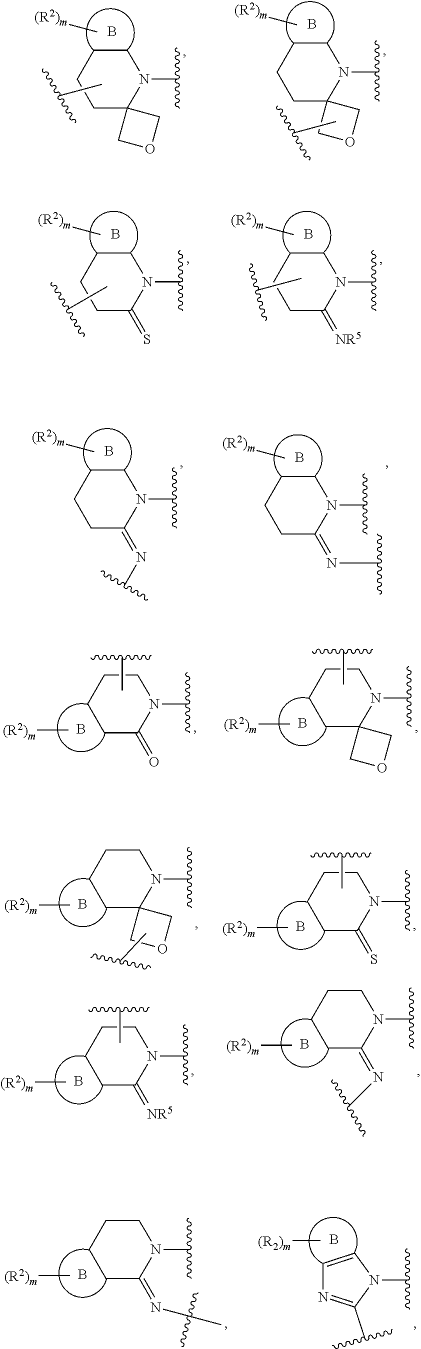

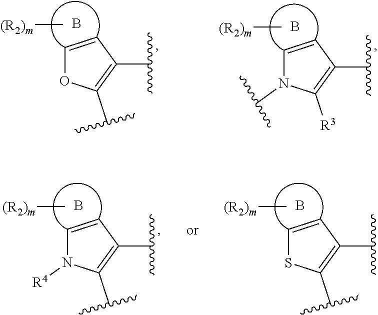

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R group.

In some embodiments, the compound of formula I-cc above is provided as a compound of formula I-cc′ or formula I-cc″:

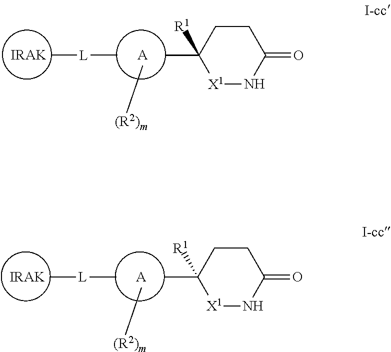

or a pharmaceutically acceptable salt thereof, wherein:
each of IRAK, Ring A, L, R¹, R², X¹, and m is as defined above.

In certain embodiments, the present invention provides a compound of formula I, wherein LBM is an E3 ubiquitin ligase (cereblon) binding moiety thereby forming a compound of formula I-dd:

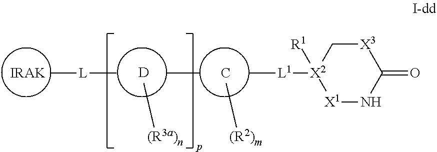

or a pharmaceutically acceptable salt thereof, wherein, L and IRAK are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

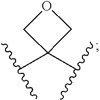 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic; Ring C is a mono- or bicyclic ring selected from

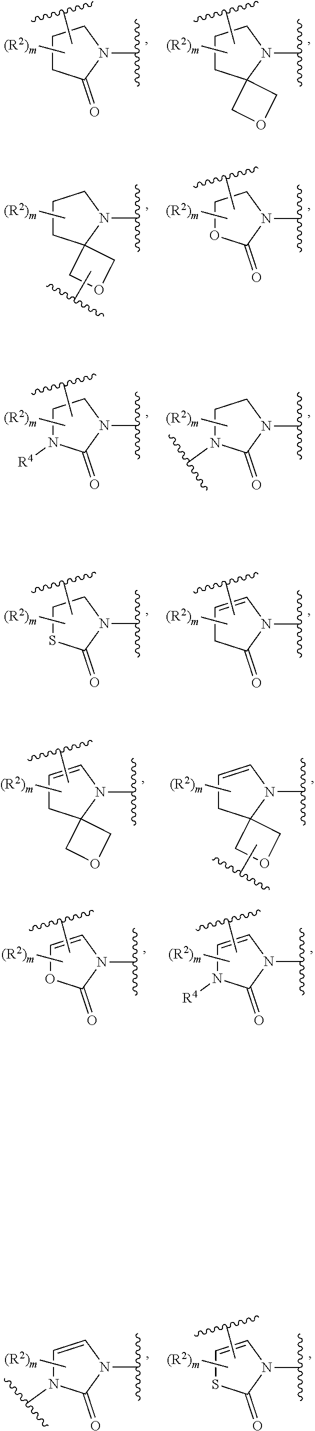

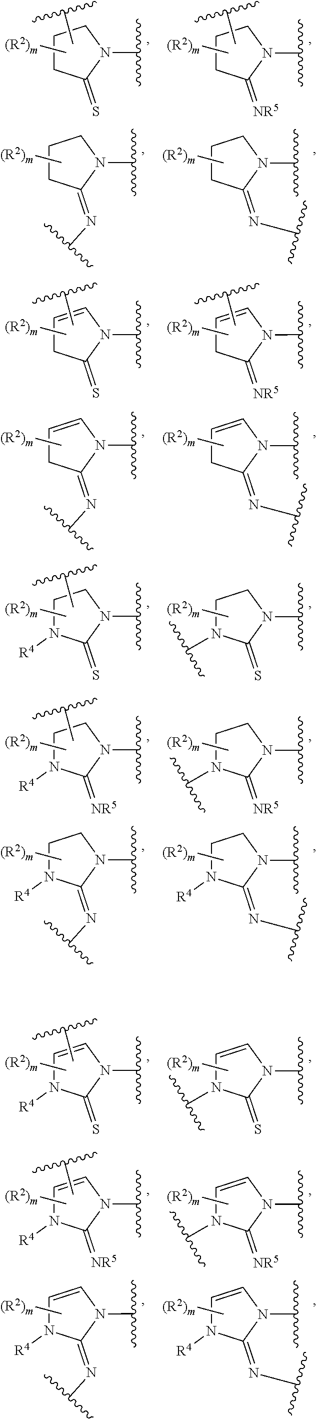

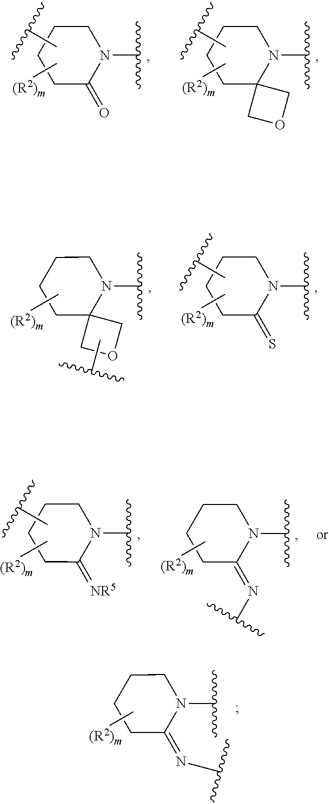

each of R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from a 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
n is 0, 1, 2, 3 or 4;
p is 0 or 1, wherein when p is 0, the bond connecting Ring C and Ring D is connected to

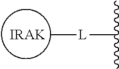

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-dd above is provided as a compound of formula I-dd′ or formula I-dd″:

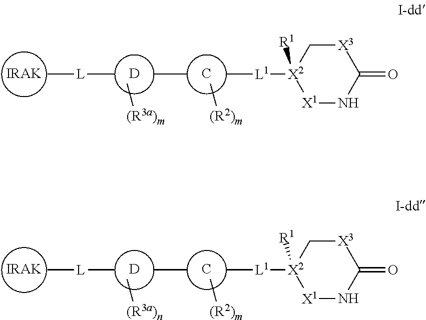

or a pharmaceutically acceptable salt thereof, wherein:
each of IRAK, Ring C, Ring D, L, L¹, R¹, R², R^{3a}, X¹, X², X³, n, m, and p is as defined above.

In certain embodiments, the present invention provides a compound of Formula I, wherein LBM is an E3 ubiquitin ligase (cereblon) binding moiety thereby forming a compound of formula I-ee:

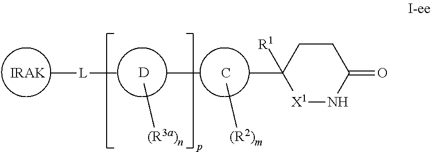

or a pharmaceutically acceptable salt thereof, wherein L and IRAK are as defined above and described in embodiments herein, and wherein:
X¹ is a bivalent moiety selected from a covalent bond, —CH₂—, —C(O)—, —C(S)—, or

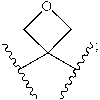 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic; Ring C is a mono- or bicyclic ring selected from

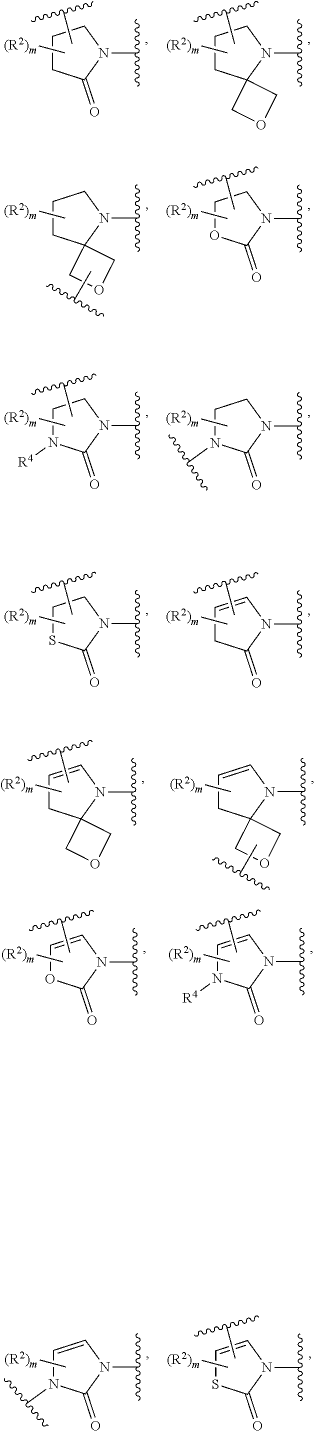

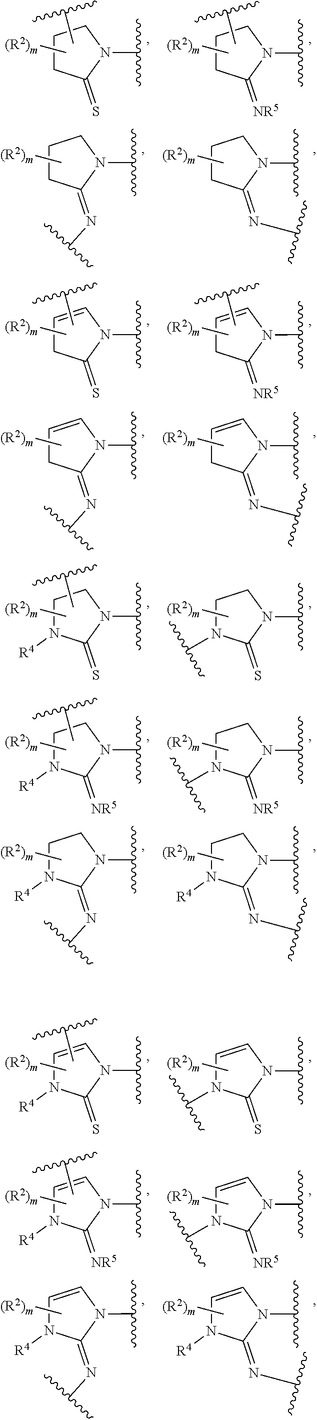

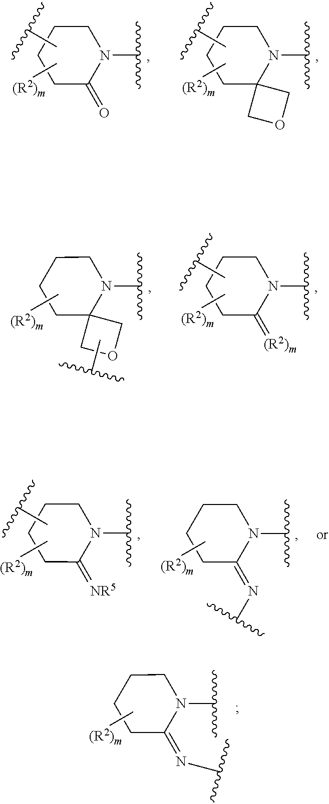

each of R² and R^{3a} is independently hydrogen, —R⁶, halogen —CN, —NO₂—OR, —SR, —NR₂, —S(O)₂NR₂, —S(O)R, —C(O)R, —C(O)OR, —C(O)NR, —C(O)N(R)OR, —OC(O)R, —OC(O)NR₂, —N(R)C(O)OR, —N(R)C(O)R, —N(R)C(O)NR₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3 or 4;
p is 0 or 1, wherein when p is 0, the bond connecting Ring C and Ring D is connected to

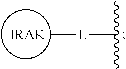

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-ee above is provided as a compound of formula I-ee′ or formula I-ee″:

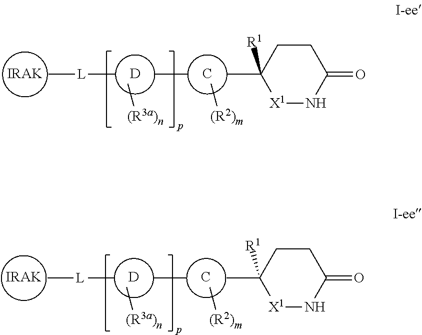

or a pharmaceutically acceptable salt thereof, wherein:
each of IRAK, Ring C, Ring D, L, R¹, R², R^{3a}, X¹, n, m, and p is as defined above.

In certain embodiments, the present invention provides a compound of formula I, wherein LBM is an E3 ubiquitin ligase (cereblon) binding moiety thereby forming a compound of formula I-ff:

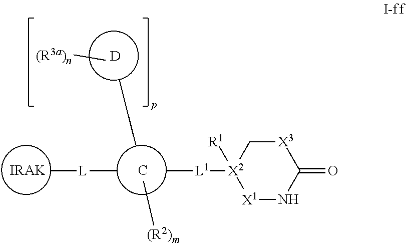

or a pharmaceutically acceptable salt thereof, wherein L and IRAK are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

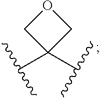 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

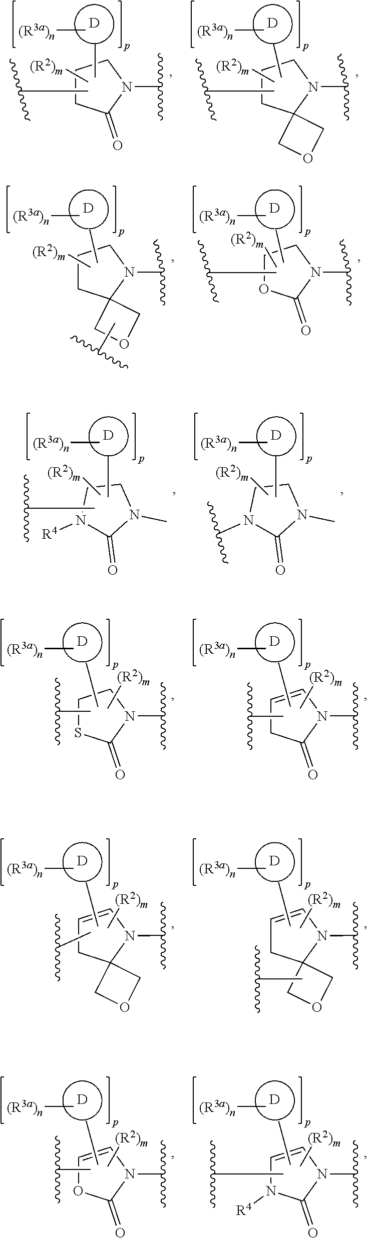

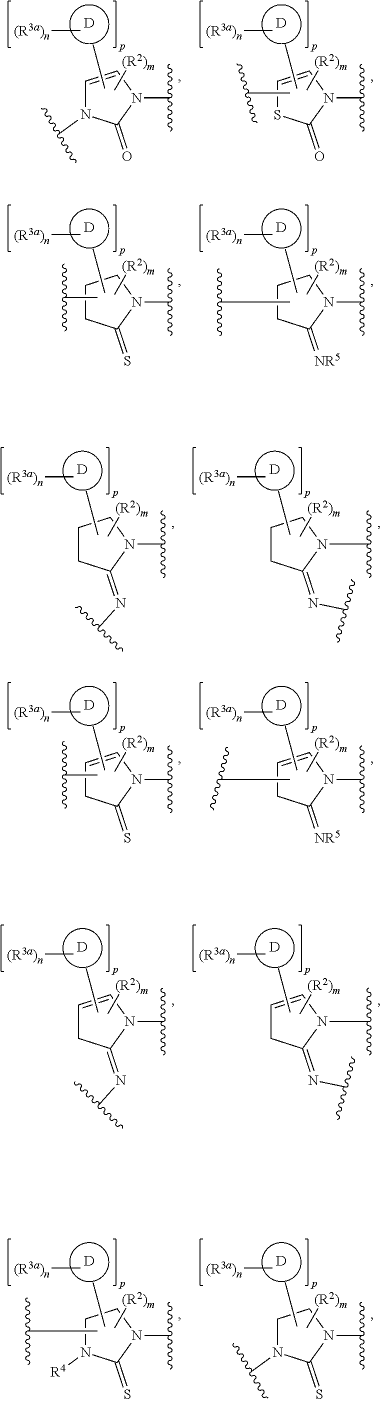

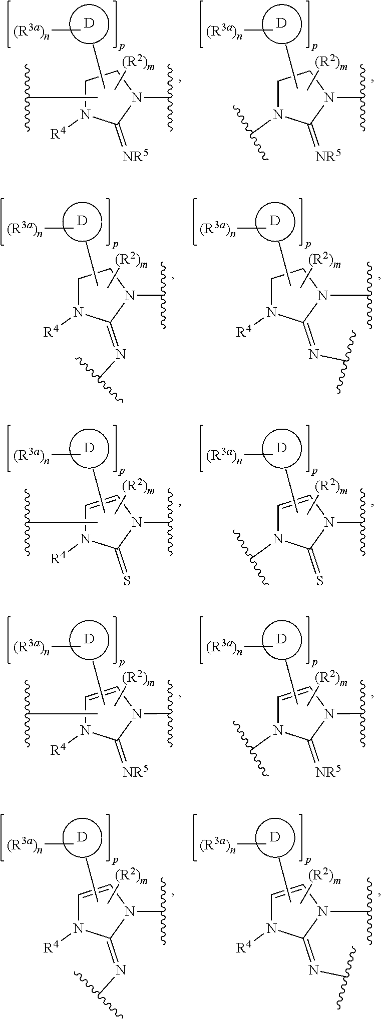

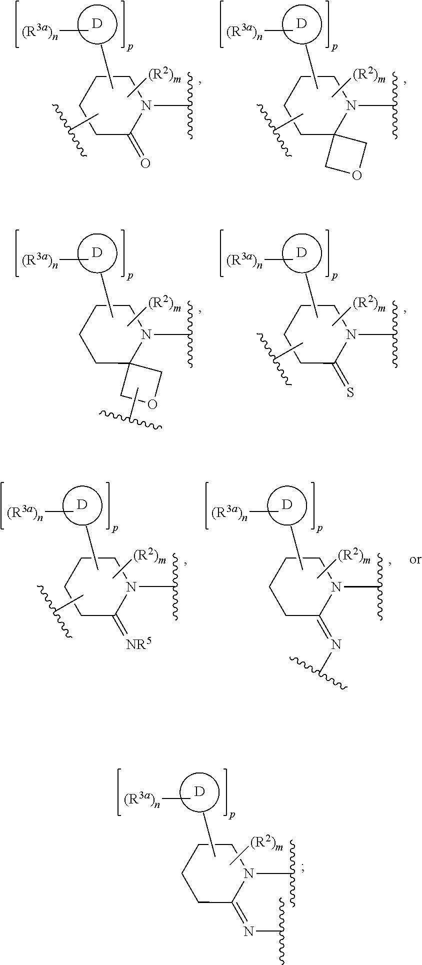

each or R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
n is 0, 1, 2, 3 or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-ff above is provided as a compound of formula I-ff′ or formula I-ff″:

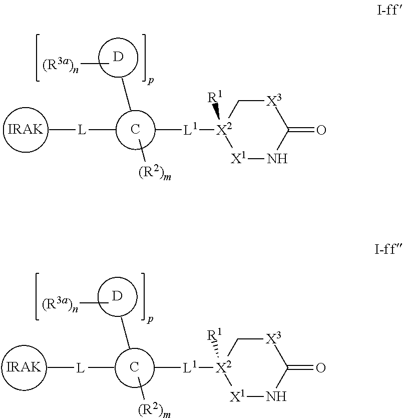

or a pharmaceutically acceptable salt thereof, wherein:
each of IRAK, Ring C, Ring D, L, L¹, R¹, R², R^{3a}, X¹, X², X³, m, n, and p is as defined above.

In certain embodiments, the present invention provides a compound of Formula I, wherein LBM is an E3 ubiquitin ligase (cereblon) binding moiety thereby forming a compound of formula I-gg:

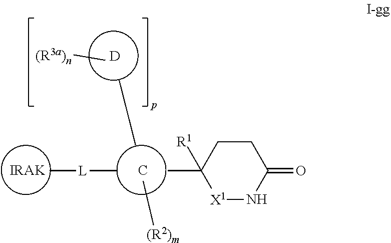

or a pharmaceutically acceptable salt thereof, wherein L and IRAK are as defined above and described in embodiments herein, and wherein:
X¹ is a bivalent moiety selected from a covalent bond, —CH₂—, —C(O)—, —C(S)—, or

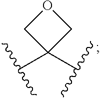 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring C is a mono- or bicyclic ring selected from

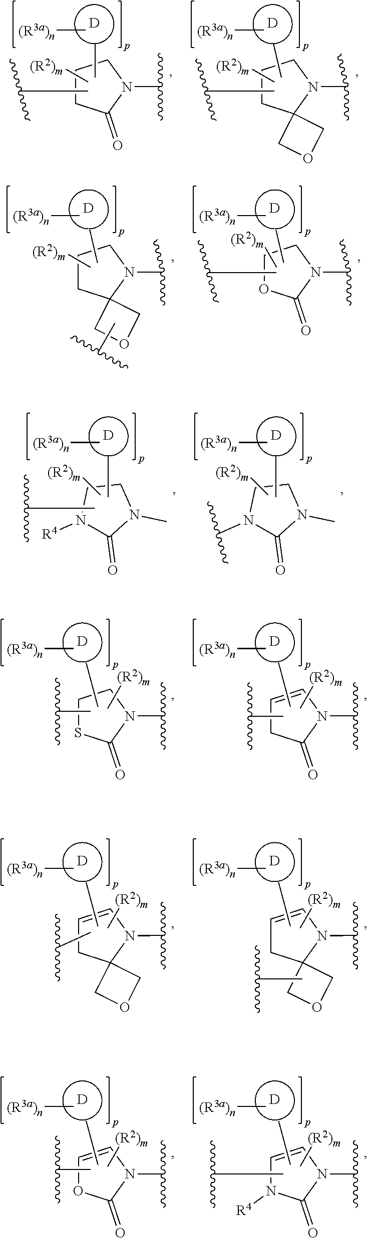

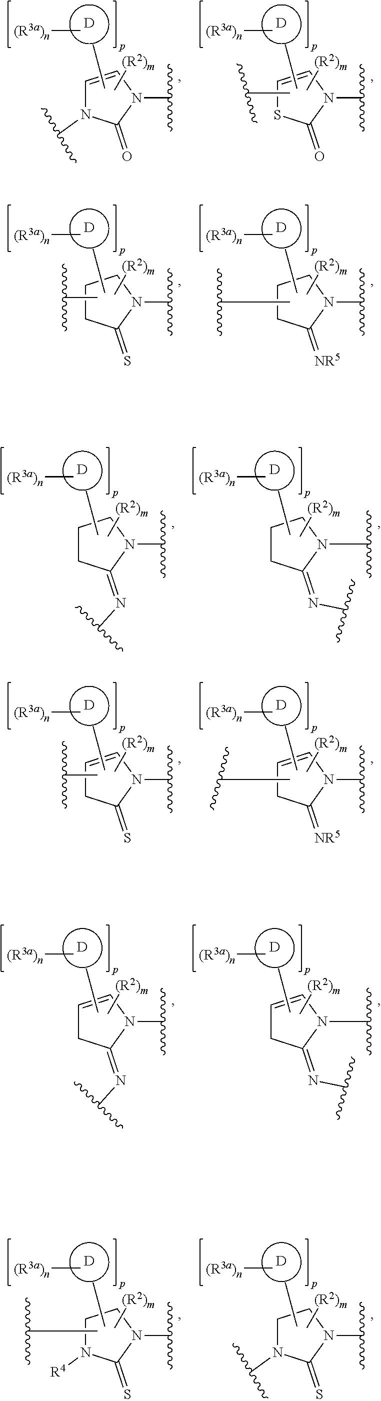

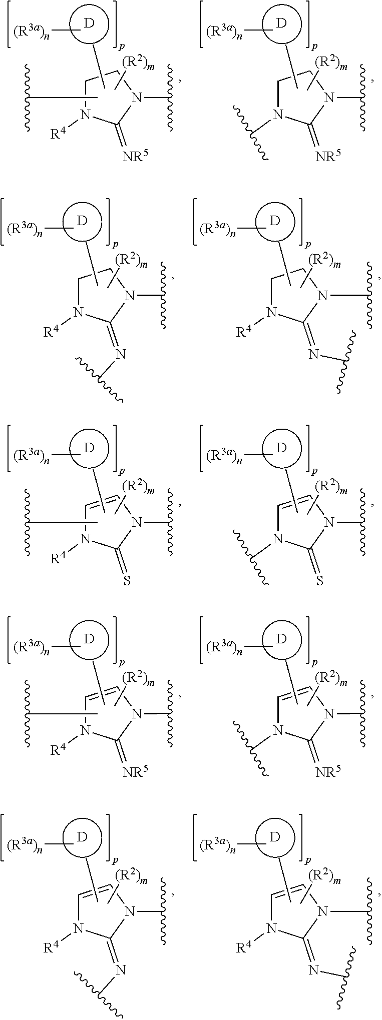

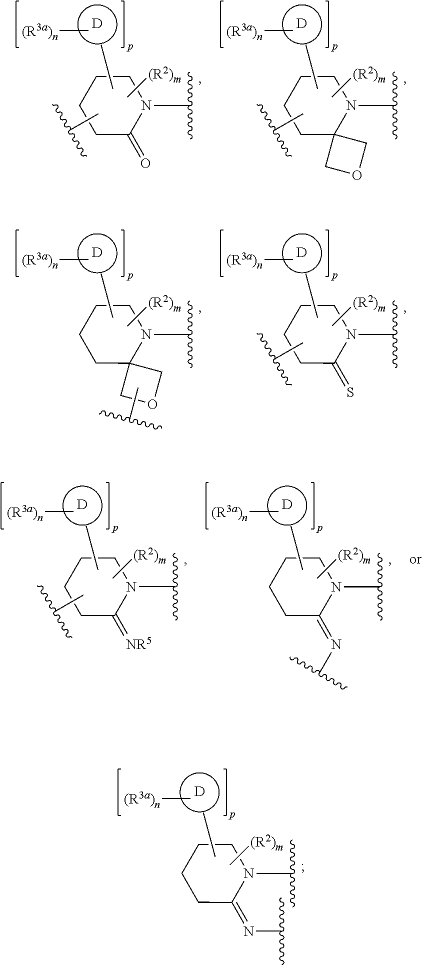

each of R², R^{3a}, and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3, or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-gg above is provided as a compound of formula I-gg′ or formula I-gg″:

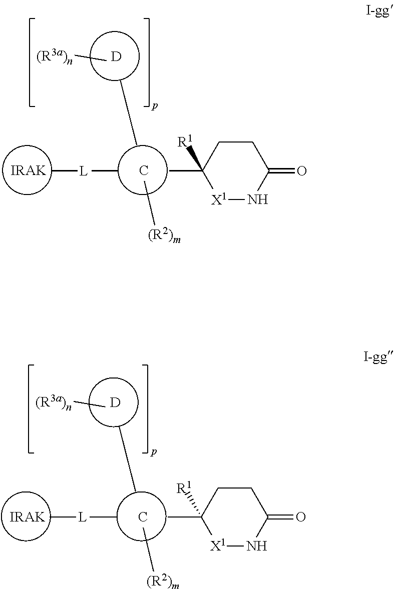

or a pharmaceutically acceptable salt thereof, wherein:
each of IRAK, Ring C, Ring D, L, R¹, R², R^{3a}, X¹, m, n, and p is as defined above.

In certain embodiments, the present invention provides a compound of Formula I, wherein LBM is an E3 ubiquitin ligase (cereblon) binding moiety thereby forming a compound of formula I-hh:

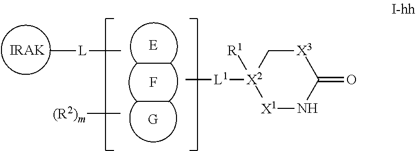

or a pharmaceutically acceptable salt thereof, wherein L and IRAK are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or;

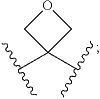 [SMILES: *C1(*)COC1]

X² is a carbon atom, nitrogen atom, or silicon atom;
X³ is a bivalent moiety selected from a covalent bond, —CR₂—, —NR—, —O—, —S—, or —SiR₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur, wherein Ring E, Ring F, and Ring G is independently and optionally substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—; and
m is 0, 1, 2, 3, 4, 5, 6, 7, 8, 9, 10, 11, 12, 13, 14, 15, or 16.

Where a point of attachment of

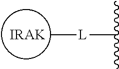

is depicted on Ring E, Ring F, or Ring G, it is intended, and one of ordinary skill in the art would appreciate, that the point of attachment of

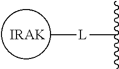

may be on any available carbon or nitrogen atom on Ring E, Ring F, or Ring G, including the ring to which Ring E or Ring G is fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G is fused to Ring F.

Where a point of attachment of

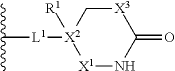

is depicted on Ring E, Ring F, or Ring G, it is intended, and one of ordinary skill in the art would appreciate, that the point of attachment of

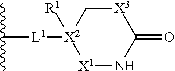

may be on any available carbon or nitrogen atom on Ring E, Ring F, or Ring G, including the carbon atom to which Ring E or Ring G is fused to Ring F.

In some embodiments, a compound of formula I-hh above is provided as a compound of formula I-hh′ or formula I-hh″.

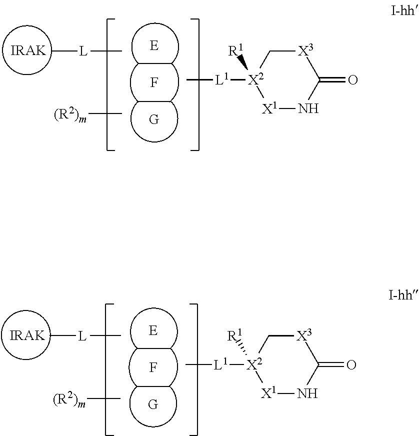

or a pharmaceutically acceptable salt thereof, wherein:
each of IRAK, Ring E, Ring F, Ring G, L, L¹, R¹, R², X¹, X², X³, and m is as defined above.

In certain embodiments, the present invention provides a compound of Formula I, wherein LBM is an E3 ubiquitin ligase (cereblon) binding moiety thereby forming a compound of formula I-hh-1 or I-hh-2:

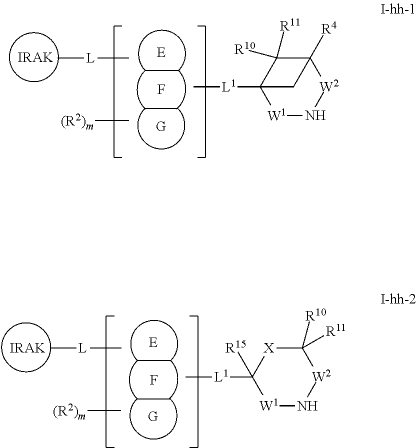

or a pharmaceutically acceptable salt thereof, wherein L and IRAK are as defined above and described in embodiments herein, and wherein:
each R² is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur, wherein Ring E, Ring F, and Ring G is independently and optionally substituted with 1-2 oxo groups;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, 4, 5, 6, 7, 8, 9, 10, 11, 12, 13, 14, 15, or 16; and
R⁴, R¹⁰, R¹¹, R¹⁵, W¹, W², and X is as defined in WO 2019/099868, the entirety of each of which is herein incorporated by reference.

Where a point of attachment of

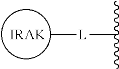

is depicted on Ring E, Ring F, or Ring G, it is intended, and one of ordinary skill in the art would appreciate, that the point of attachment of

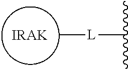

may be on any available carbon or nitrogen atom on Ring E, Ring F, or Ring G, including the ring to which Ring E or Ring G is fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G is fused to Ring F.

Where a point of attachment of

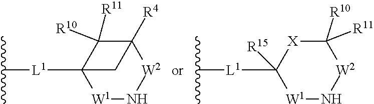

is depicted on Ring E, Ring F, or Ring G, it is intended, and one of ordinary skill in the art would appreciate, that the point of attachment of

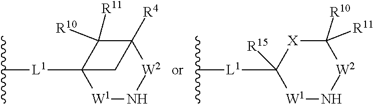

may be on any available carbon or nitrogen atom on Ring E, Ring F, or Ring G, including the carbon atom to which Ring E or Ring G is fused to Ring F.

In certain embodiments, the present invention provides a compound of Formula I, wherein LBM is an E3 ubiquitin ligase (cereblon) binding moiety thereby forming a compound of formula I-ii:

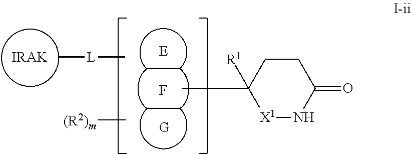

or a pharmaceutically acceptable salt thereof, wherein L and IRAK are as defined above and described in embodiments herein, and wherein:
X¹ is a bivalent moiety selected from a covalent bond, —CH₂—, —C(O)—, —C(S)—, or

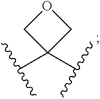 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl containing 0-3 nitrogens, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-3 heteroatoms independently selected from nitrogen, oxygen or sulfur, wherein Ring E, Ring F, and Ring G is independently and optionally substituted with 1-2 oxo groups; and
m is 0, 1, 2, 3, or 4.

Where a point of attachment of

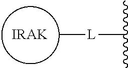

is depicted on Ring E, Ring F, or Ring G, it is intended, and one of ordinary skill in the art would appreciate, that the point of attachment of

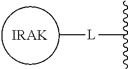

may be on any available carbon or nitrogen atom on Ring E, Ring F, or Ring G, including the ring to which Ring E or Ring G is fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G is fused to Ring F.

In some embodiments, a compound of formula I-ii above is provided as a compound of formula I-ii′ or formula I-ii″

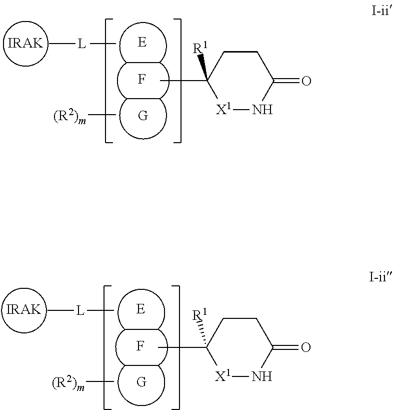

or a pharmaceutically acceptable salt thereof, wherein:
each of IRAK, L, Ring E, Ring F, Ring G, L, R¹, R², X¹, and m is as defined above.

In certain embodiments, the present invention provides a compound of Formula I, wherein LBM is an E3 ubiquitin ligase (cereblon) binding moiety thereby forming a compound of formula I-jj:

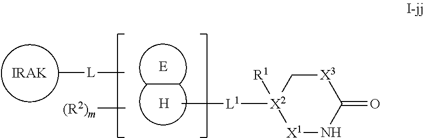

or a pharmaceutically acceptable salt thereof, wherein L and IRAK are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

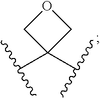 [SMILES: *C1(*)COC1]

X² is a carbon atom, nitrogen atom, or silicon atom;
X³ is a bivalent moiety selected from a covalent bond, —CR₂—, —NR—, —O—, —S—, or —SiR₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring H is a fused ring selected from a 7-9 membered saturated or partially unsaturated carbocyclyl or heterocyclyl ring with 1-3 heteroatoms independently selected from boron, nitrogen, oxygen, silicon, or sulfur, wherein Ring E is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, or 4.

Where a point of attachment of

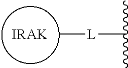

is depicted on Ring E or Ring H, it is intended, and one of ordinary skill in the art would appreciate, that the point of attachment of

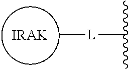

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

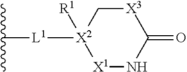

is depicted on Ring E and Ring H, it is intended, and one of ordinary skill in the art would appreciate, that the point of attachment of

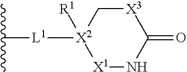

may be on any available carbon or nitrogen atom on Ring E or Ring H including the carbon atom to which Ring E and Ring H are fused.

In some embodiments, a compound of formula I-jj above is provided as a compound of formula I-jj′ or formula I-jj″:

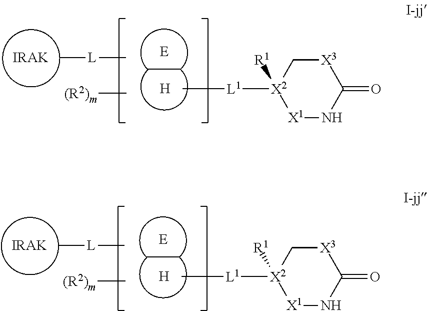

or a pharmaceutically acceptable salt thereof, wherein:
each of IRAK, Ring E, Ring H, L, L¹, R¹, R², X¹, X², X³, and m is as defined above.

In certain embodiments, the present invention provides a compound of Formula I, wherein LBM is an E3 ubiquitin ligase (cereblon) binding moiety thereby forming a compound of formula I-kk:

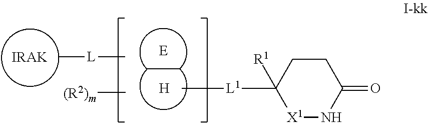

or a pharmaceutically acceptable salt thereof, wherein L and IRAK are as defined above and described in embodiments herein, and wherein:
X¹ is a bivalent moiety selected from a covalent bond, —CH₂—, —C(O)—, —C(S)—, or

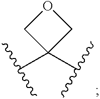 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring H is a ring selected from a 7-9 membered saturated or partially unsaturated carbocyclyl or heterocyclyl ring with 1-3 heteroatoms independently selected from boron, nitrogen, oxygen, silicon, or sulfur, wherein Ring E is optionally further substituted with 1-2 oxo groups; and
m is 0, 1, 2, 3, or 4.

Where a point of attachment of

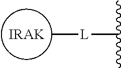

is depicted on Ring E or Ring H, it is intended, and one of ordinary skill in the art would appreciate, that the point of attachment of

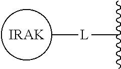

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

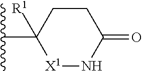

is depicted on Ring E and Ring H, it is intended, and one of ordinary skill in the art would appreciate, that the point of attachment of

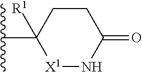

may be on any available carbon or nitrogen atom on Ring E or Ring H including the carbon atom to which Ring E and Ring H are fused.

In some embodiments, a compound of formula I-kk above is provided as a compound of formula I-kk′ or formula I-kk″:

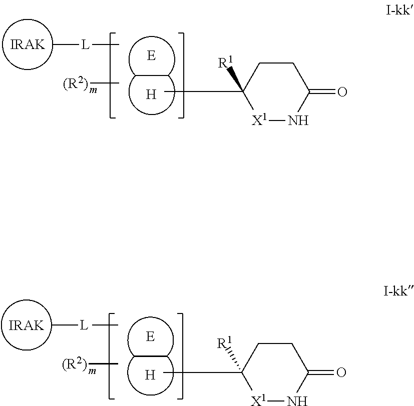

or a pharmaceutically acceptable salt thereof, wherein:
each of IRAK, Ring E, Ring H, L, R¹, R², X¹, and m is as defined above.

In certain embodiments, the present invention provides a compound of Formula I, wherein LBM is an E3 ubiquitin ligase (cereblon) binding moiety thereby forming a compound of formula I-11:

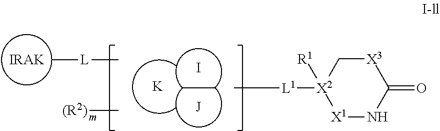

or a pharmaceutically acceptable salt thereof, wherein L and IRAK are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

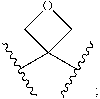 [SMILES: *C1(*)COC1]

X² is a carbon atom, nitrogen atom, or silicon atom;
X³ is a bivalent moiety selected from a covalent bond, —CR₂—, —NR—, —O—, —S—, or —SiR₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring K is a fused ring selected from a 7-12 membered saturated or partially unsaturated carbocyclyl or heterocyclyl ring with 1-3 heteroatoms independently selected from boron, nitrogen, oxygen, silicon, or sulfur, wherein Ring H is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—; and
m is 0, 1, 2, 3, or 4.

Where a point of attachment of

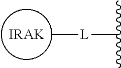

is depicted on Ring I, Ring J, and Ring K, it is intended, and one of ordinary skill in the art would appreciate, that the point of attachment of

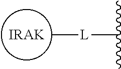

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

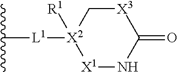

is depicted on Ring I, Ring J, and Ring K, it is intended, and one of ordinary skill in the art would appreciate, that the point of attachment of

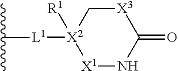

may be on any available carbon or nitrogen atom on Ring I, Ring J, or Ring K, including the carbon atom to which Ring I, Ring J, and Ring K are fused.

In some embodiments, a compound of formula I-ll above is provided as a compound of formula I-ll′ or formula I-ll″:

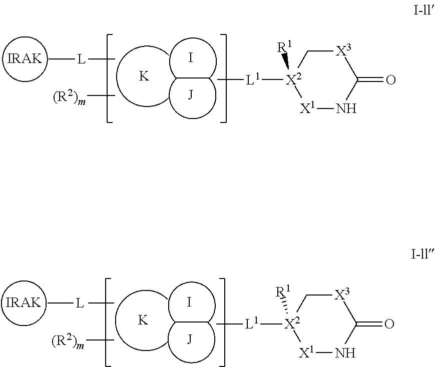

or a pharmaceutically acceptable salt thereof, wherein:
each of IRAK, Ring I, Ring J, Ring K, L, L¹, R¹, R², X¹, X², X³, and m is as defined above.

In certain embodiments, the present invention provides a compound of formula I-mm:

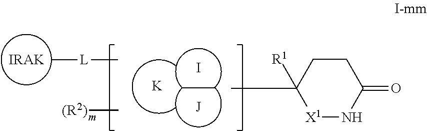

or a pharmaceutically acceptable salt thereof, wherein L and IRAK are as defined above and described in embodiments herein, and wherein:
X¹ is a bivalent moiety selected from a covalent bond, —CH₂—, —C(O)—, —C(S)—, or

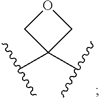 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring K is a fused ring selected from a 7-12 membered saturated or partially unsaturated carbocyclyl or heterocyclyl ring with 1-3 heteroatoms independently selected from boron, nitrogen, oxygen, silicon, or sulfur, wherein Ring H is optionally further substituted with 1-2 oxo groups; and
m is 0, 1, 2, 3, or 4.

Where a point of attachment of

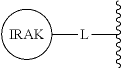

is depicted on Ring I, Ring J, and Ring K, it is intended and one of ordinary skill in the art would appreciate, that the point of attachment of

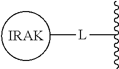

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

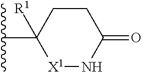

is depicted on Ring I, Ring J, and Ring K, it is intended, and one of ordinary skill in the art would appreciate, that the point of attachment of

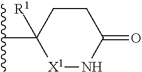

may be on any available carbon or nitrogen atom on Ring I, Ring J, or Ring K, including the carbon atom to which Ring I, Ring J, and Ring K are fused.

In some embodiments, a compound of formula I-mm above is provided as a compound of formula I-mm′ or formula I-mm″:

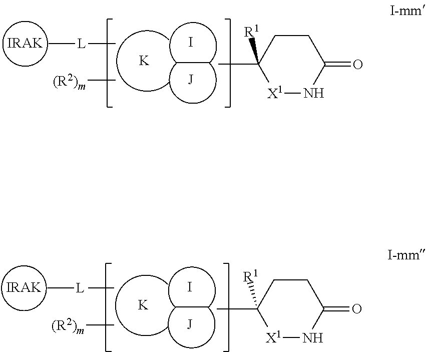

or a pharmaceutically acceptable salt thereof, wherein:
each of IRAK, Ring I, Ring J, Ring K, L, R¹, R², X¹, and m is as defined above.

As described above, in another aspect, the present invention provides a compound of Formula

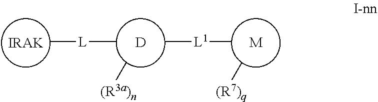

or a pharmaceutically acceptable salt thereof, wherein:
Ring M is selected from

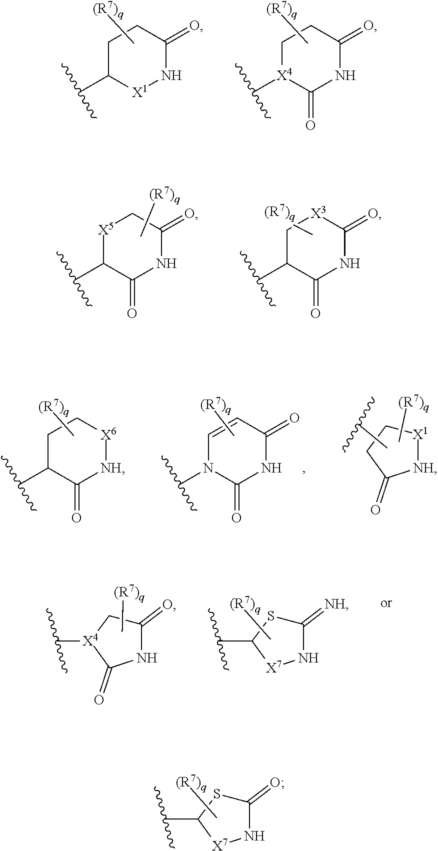

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

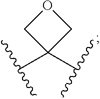 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

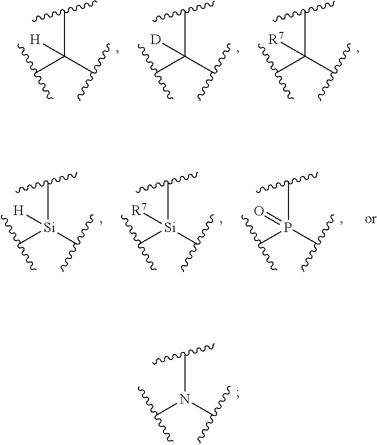

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH—)₂R, —SiR₃, or an optionally substituted C₁₋₄ aliphatic; or
   R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur;
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
n is 0, 1, 2, 3, or 4; and
q is 0, 1, 2, 3, or 4.

As defined above and described herein, each of X¹, X⁶, and X⁷ is independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

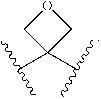 [SMILES: *C1(*)COC1]

In some embodiments, each of X¹, X⁶, and X⁷ is independently a covalent bond. In some embodiments, each of X¹, X⁶, and X⁷ is independently —CH₂—. In some embodiments, each of X¹, X⁶, and X⁷ is independently —CR₂—. In some embodiments, each of X¹, X⁶, and X⁷ is independently —C(O)—. In some embodiments, each of X¹, X⁶, and X⁷ is independently —C(S)—. In some embodiments, each of X¹, X⁶, and X⁷ is independently —CH(R)—. In some embodiments, each of X¹, X⁶, and X⁷ is independently —CH(CF₃)—. In some embodiments, each of X¹, X⁶, and X⁷ is independently —P(O)(OR)—. In some embodiments, each of X¹, X⁶, and X⁷ is independently —P(O)(R)—. In some embodiments, each of X¹, X⁶, and X⁷ is independently —P(O)NR₂—. In some embodiments, each of X¹, X⁶, and X⁷ is independently —S(O)—In some embodiments, each of X¹, X⁶, and X⁷ is independently —S(O)₂—. In some embodiments, each of X¹, X⁶, and X⁷ is independently

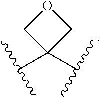 [SMILES: *C1(*)COC1]

In some embodiments, each of X¹, X⁶, and X⁷ is independently selected from those depicted in Table 1 below.

As defined above and described herein, X² is a carbon atom, nitrogen atom, or silicon atom.

In some embodiments, X² is a carbon atom. In some embodiments, X² is a nitrogen atom. In some embodiments, X² is a silicon atom.

In some embodiments, X² is selected from those depicted in Table 1 below.

As defined above and described herein, X³ is a bivalent moiety selected from —CH₂—, —CR₂—, —NR—, —CF₂—, —CHF—, —S—, —CH(R)—, —SiR₂—, or —O—.

In some embodiments, each of X³ and X⁵ is independently —CH₂—. In some embodiments, each of X³ and X⁵ is independently —CR₂—. In some embodiments, each of X³ and X⁵ is independently —NR—. In some embodiments, each of X³ and X⁵ is independently —CF₂—. In some embodiments, each of X³ and X⁵ is independently —CHF—. In some embodiments, each of X³ and X⁵ is independently —S—. In some embodiments, each of X³ and X⁵ is independently —CH(R)—. In some embodiments, each of X³ and X⁵ is independently —SiR₂—. In some embodiments, each of X³ and X⁵ is independently —O—.

In some embodiments, each of X³ and X⁵ is independently selected from those depicted in Table 1 below.

As defined above and described herein, X⁴ is a trivalent moiety selected from

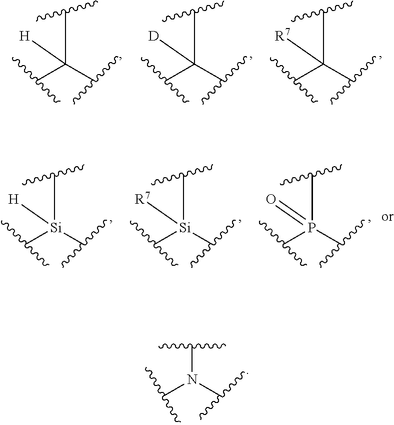

In some embodiments, X⁴ is

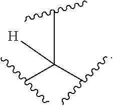 [SMILES: *C(*)*]

In some embodiments, X⁴ is

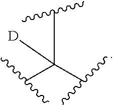 [SMILES: *C(*)(*)[2H]]

In some embodiments, X⁴ is

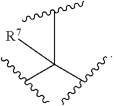

In some embodiments, X⁴ is

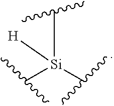 [SMILES: *[SiH](*)*]

In some embodiments, X⁴ is

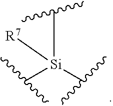

In some embodiments, X⁴ is

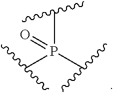 [SMILES: *P(*)(*)=O]

In some embodiments, X⁴ is

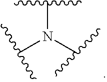 [SMILES: *N(*)*]

In some embodiments, X⁴ is selected from those depicted in Table 1 below.

As defined above and described herein, R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, an optionally substituted C₁₋₄ aliphatic, or R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, or sulfur.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —P(O)(OR)₂. In some embodiments, R¹ is —P(O)(NR₂)OR. In some embodiments, R¹ is —P(O)(NR₂)₂. In some embodiments, R¹ is —Si(OH)₂R. In some embodiments, R¹ is —Si(OH)R₂. In some embodiments, R¹ is —SiR₃. In some embodiments, R¹ is an optionally substituted C₁₋₄ aliphatic. In some embodiments, R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, or sulfur.

In some embodiments, R¹ is selected from those depicted in Table 1 below.

As defined above and described herein, each R is independently hydrogen, deuterium, or an optionally substituted group selected from C₁₋₆ aliphatic, phenyl, a 4-7 membered saturated or partially unsaturated heterocyclic having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur, or two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is hydrogen. In some embodiments, R is deuterium. In some embodiments, R is optionally substituted C₁₋₆ aliphatic. In some embodiments, R is optionally substituted phenyl. In some embodiments, R is optionally substituted 4-7 membered saturated or partially unsaturated heterocyclic having 1-3 heteroatoms independently selected from boron, nitrogen, oxygen, silicon, and sulfur. In some embodiments, R is optionally substituted 5-6 membered heteroaryl ring having 1-4 heteroatoms independently selected from boron, nitrogen, oxygen, silicon, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is selected from those depicted in Table 1 below.

As defined above and described herein, each of R² and R^{3a} is independently hydrogen, deuterium, —R⁶, halogen, —CN, —NO₂, —OR, —Si(OH)₂R, —Si(OH)R₂, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R.

In some embodiments, R² and/or R^{3a} is hydrogen. In some embodiments, R² and/or R^{3a} is deuterium. In some embodiments, R² and/or R^{3a} is —R⁶. In some embodiments, R² and/or R^{3a} is halogen. In some embodiments, R² and/or R^{3a} is —CN. In some embodiments, R² and/or R^{3a} is —NO₂. In some embodiments, R² and/or R^{3a} is —OR. In some embodiments, R² and/or R^{3a} is —Si(OH)₂R. In some embodiments, R² and/or R^{3a} is —Si(OH)R₂. In some embodiments, R² and/or R^{3a} is —SR. In some embodiments, R² and/or R^{3a} is —NR₂. In some embodiments, R² and/or R^{3a} is —SiR₃. In some embodiments, R² and/or R^{3a} is —S(O)₂R. In some embodiments, R² and/or R^{3a} is —S(O)₂NR₂. In some embodiments, R² and/or R^{3a} is —S(O)R. In some embodiments, R² and/or R^{3a} is —C(O)R. In some embodiments, R² and/or R^{3a} is —C(O)OR. In some embodiments, R² and/or R^{3a} is —C(O)NR₂. In some embodiments, R² and/or R^{3a} is —C(O)N(R)OR. In some embodiments, R² and/or R^{3a} is —C(R)₂N(R)C(O)R. In some embodiments, R² and/or R^{3a} is —C(R)₂N(R)C(O)NR₂. In some embodiments, R² and/or R^{3a} is —OC(O)R. In some embodiments, R² and/or R^{3a} is —OC(O)NR₂. In some embodiments, R² and/or R^{3a} is —OP(O)R₂. In some embodiments, R² and/or R^{3a} is —OP(O)(OR)₂. In some embodiments, R² and/or R^{3a} is —OP(O)(OR)NR₂. In some embodiments, R² and/or R^{3a} is —OP(O)(NR₂)₂—. In some embodiments, R² and/or R^{3a} is —N(R)C(O)OR. In some embodiments, R² and/or R^{3a} is —N(R)C(O)R. In some embodiments, R² and/or R^{3a} is —N(R)C(O)NR₂. In some embodiments, R² and/or R^{3a} is —NP(O)R². In some embodiments, R² and/or R^{3a} is —N(R)P(O)(OR)₂. In some embodiments, R² and/or R^{3a} is —N(R)P(O)(OR)NR₂. In some embodiments, R² and/or R^{3a} is —N(R)P(O)(NR₂)₂. In some embodiments, R² and/or R^{3a} is —N(R)S(O)₂R.

In some embodiments, R² and/or R^{3a} is —OH. In some embodiments, R² and/or R^{3a} is —NH₂. In some embodiments, R² and/or R^{3a} is —CH₂NH₂. In some embodiments, R² and/or R^{3a} is —CH₂NHCOMe. In some embodiments, R² and/or R^{3a} is —CH₂NHCONHMe. In some embodiments, R² and/or R^{3a} is —NHCOMe. In some embodiments, R² and/or R^{3a} is —NHCONHEt. In some embodiments, R² and/or R^{3a} is —SiMe₃. In some embodiments, R² and/or R^{3a} is —SiMe₂OH. In some embodiments, R² and/or R^{3a} is —SiMe(OH)₂. In some embodiments R² and/or R^{3a} is

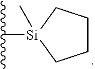 [SMILES: *[Si]1(C)CCCC1]

In some embodiments, R² and/or R^{3a} is Br. In some embodiments, R² and/or R^{3a} is Cl. In some embodiments, R² and/or R^{3a} is F. In some embodiments, R² and/or R^{3a} is Me. In some embodiments, R² and/or R^{3a} is —NHMe. In some embodiments, R² and/or R^{3a} is —NMe₂. In some embodiments, R² and/or R^{3a} is —NHCO₂Et. In some embodiments, R² and/or R^{3a} is —CN. In some embodiments, R² and/or R^{3a} is —CH₂Ph. In some embodiments, R² and/or R^{3a} is —NHCO₂tBu. In some embodiments, R² and/or R^{3a} is —CO₂tBu. In some embodiments, R² and/or R^{3a} is —OMe. In some embodiments, R² and/or R^{3a} is —CF₃.

In some embodiments, R² and R^{3a} is selected from those depicted in Table 1 below.

As defined above and described herein, R³ is hydrogen, deuterium, halogen, —CN, —NO₂, —OR, —NR₂, —SR, —S(O)₂R, —S(O)₂NR₂, —S(O)R, —C(O)R, —C(O)OR, —C(O)NR₂, —C(O)NR(OR), —OC(O)R, —OC(O)NR₂, —OP(O)(OR)₂, —OP(O)(NR₂)₂, —OP(O)(OR)NR₂, —N(R)C(O)R, —N(R)C(O)OR, —N(R)C(O)NR₂, —N(R)S(O)₂R, —N(R)S(O)₂NR₂, —N(R)P(O)(OR)₂, —N(R)P(O)(OR)NR₂, —P(O)(OR)₂, —P(O)(NR₂)OR, —P(O)(NR₂)₂, —Si(OH)₂R, —Si(OH)(R)₂, or —Si(R)₃.

In some embodiments, R³ is hydrogen. In some embodiments, R³ is deuterium. In some embodiments, R³ is halogen. In some embodiments, R³ is —CN. In some embodiments, R³ is —NO₂. In some embodiments, R³ is —OR. In some embodiments, R³ is —NR₂. In some embodiments, R³ is —SR. In some embodiments, R³ is —S(O)₂R. In some embodiments, R³ is —S(O)₂NR₂. In some embodiments, R³ is —S(O)R. In some embodiments, R³ is —C(O)R. In some embodiments, R³ is —C(O)OR. In some embodiments, R³ is —C(O)NR₂. In some embodiments, R³ is —C(O)NR(OR). In some embodiments, R³ is —OC(O)R. In some embodiments, R³ is —OC(O)NR₂. In some embodiments, R³ is —OP(O)(OR)₂. In some embodiments, R³ is —OP(O)(NR₂)₂. In some embodiments, R³ is —OP(O)(OR)NR₂. In some embodiments, R³ is —N(R)C(O)R. In some embodiments, R³ is —N(R)C(O)OR. In some embodiments, R³ is —N(R)C(O)NR₂. In some embodiments, R³ is —N(R)S(O)₂R. In some embodiments, R³ is —N(R)S(O)₂NR₂. In some embodiments, R³ is —N(R)P(O)(OR)₂. In some embodiments, R³ is —N(R)P(O)(OR)NR₂. In some embodiments, R³ is —P(O)(OR)₂. In some embodiments, R³ is —P(O)(NR₂)OR. In some embodiments, R³ is —P(O)(NR₂)₂. In some embodiments, R³ is —Si(OH)₂R. In some embodiments, R³ is —Si(OH)(R)₂. In some embodiments, R³ is —Si(R)₃.

In some embodiments, R³ is methyl. In some embodiments, R³ is —OCH₃. In some embodiments, R³ is chloro.

In some embodiments, R³ is selected from those depicted in Table 1.

As defined above and described herein, each R⁴ is independently hydrogen, deuterium, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —P(O)(OR)₂, —P(O)(NR₂)OR, or —P(O)(NR₂)₂.

In some embodiments, R⁴ is hydrogen. In some embodiments, R⁴ is —R⁶. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R)OR. In some embodiments, R⁴ is —OC(O)R. In some embodiments, R⁴ is —OC(O)NR₂. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)S(O)₂R. In some embodiments, R⁴ is —P(O)(OR)₂. In some embodiments, R⁴ is —P(O)(NR₂)OR. In some embodiments, R⁴ is —P(O)(NR₂)₂.

In some embodiments, R⁴ is methyl. In some embodiments, R⁴ is ethyl. In some embodiments, R⁴ is cyclopropyl.

In some embodiments, R⁴ is selected from those depicted in Table 1.

As defined above and described herein, R⁵ is hydrogen, deuterium, an optionally substitute C₁. 4 aliphatic, or —CN.

In some embodiments, R⁵ is hydrogen. In some embodiments, R⁵ is deuterium. In some embodiments, R⁵ is an optionally substituted C₁₋₄ aliphatic. In some embodiments, R⁵ is —CN.

In some embodiments, R⁵ is selected from those depicted in Table 1.

As defined above and described herein, each R⁶ is independently an optionally substituted group selected from C₁₋₆ aliphatic, phenyl, a 4-7 membered saturated or partially unsaturated heterocyclic ring having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur.

In some embodiments, R⁶ is an optionally substituted C₁₋₆ aliphatic. In some embodiments, R⁶ is an optionally substituted phenyl. In some embodiments, R⁶ is an optionally substituted 4-7 membered saturated or partially unsaturated heterocyclic ring having 1-3 heteroatoms independently selected from boron, nitrogen, oxygen, silicon, and sulfur. In some embodiments, R⁶ is an optionally substituted 5-6 membered heteroaryl ring having 1-4 heteroatoms independently selected from boron, nitrogen, oxygen, silicon, and sulfur.

In some embodiments, R⁶ is selected from those depicted in Table 1.

As defined generally above, each R⁷ is independently hydrogen, deuterium, halogen, —CN, —OR, —SR, —S(O)R, —S(O)₂R, —N(R)₂, —P(O)(R)₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic, or R¹ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur, or two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur, or two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur.

In some embodiments, R⁷ is hydrogen. In some embodiments, R⁷ is deuterium. In some embodiments, R⁷ is halogen. In some embodiments, R⁷ is —CN. In some embodiments, R⁷ is —OR. In some embodiments, R⁷ is —SR. In some embodiments, R⁷ is —S(O)R. In some embodiments, R⁷ is —S(O)₂R. In some embodiments, R⁷ is —NR₂. In some embodiments, R⁷ is —Si(R)₃. In some embodiments, R⁷ is —P(O)(R)₂. In some embodiments, R⁷ is —P(O)(OR)₂. In some embodiments, R⁷ is —P(O)(NR₂)OR. In some embodiments, R⁷ is —P(O)(NR₂)₂. In some embodiments, R⁷ is —Si(OH)R₂. In some embodiments, R⁷ is —Si(OH)₂R. In some embodiments, R⁷ is an optionally substituted C₁₋₄ aliphatic. In some embodiments, R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur. In some embodiments, two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur. In some embodiments, two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur. In some embodiments, two R⁷ groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, or sulfur.

In some embodiments, R⁷ is selected from hydrogen, halogen, —CN, —OR, —NR₂, or C₁₋₄ alkyl. In some embodiments, R⁷ is selected from hydrogen, halogen, —CN, or C₁₋₄ alkyl. In some embodiments, R⁷ is fluoro. In some embodiments, two R⁷ groups on the same carbon are optionally taken together with their intervening atoms to form a 3- or 4-membered spiro fused ring.

In some embodiments, R⁷ is selected from those depicted in Table 1 below.

As defined above and described herein, Ring A is a bi- or tricyclic ring selected from

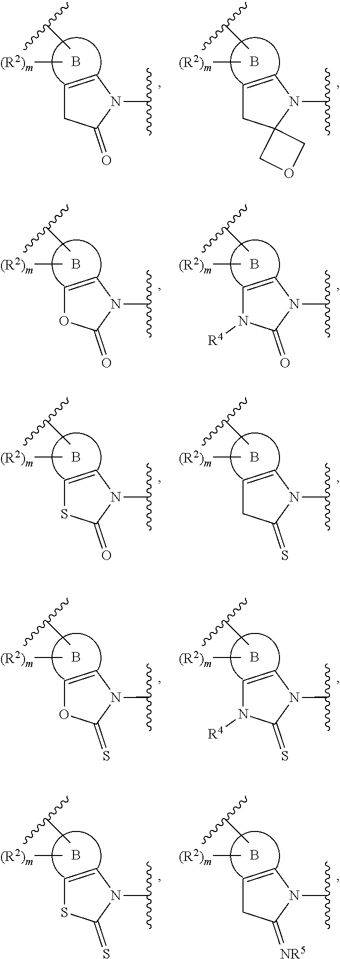

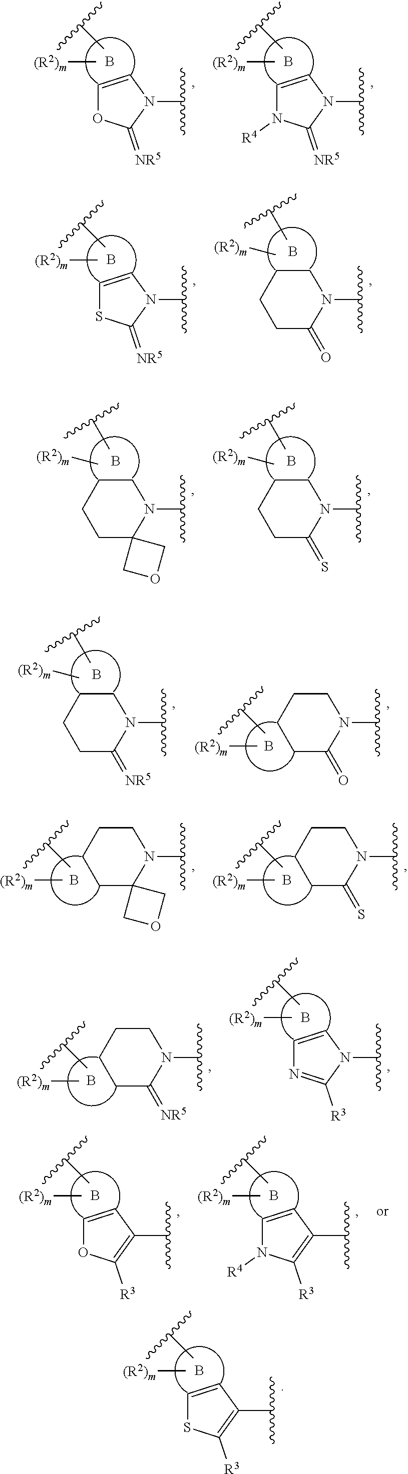

In some embodiments, Ring A is

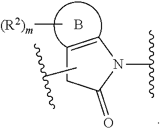

In some embodiments, Ring A is

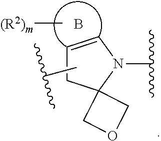

In some embodiments, Ring A is

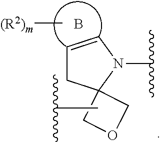

In some embodiments, Ring A is

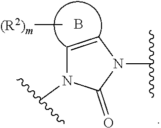

In some embodiments, Ring A is

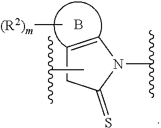

In some embodiments, Ring A is

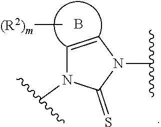

In some embodiments, Ring A is

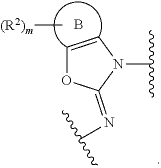

In some embodiments, Ring A is

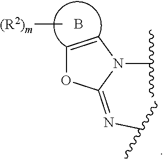

In some embodiments, Ring A is

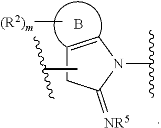

In some embodiments, Ring A is

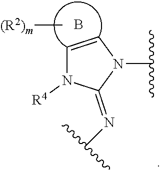

In some embodiments, Ring A is

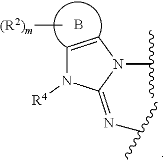

In some embodiments, Ring A is

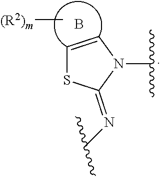

In some embodiments, Ring A is

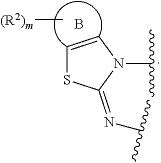

In some embodiments, Ring A is

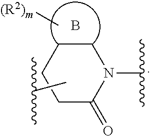

In some embodiments, Ring A is

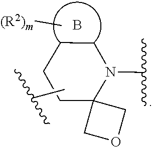

In some embodiments, Ring A is

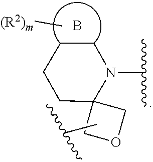

In some embodiments, Ring A is

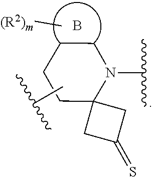

In some embodiments, Ring A is

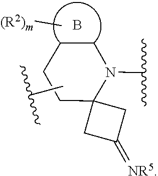

In some embodiments, Ring A is

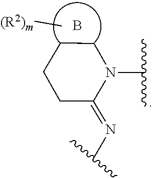

In some embodiments, Ring A is

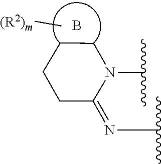

In some embodiments, Ring A is

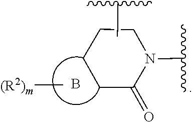

In some embodiments, Ring A is

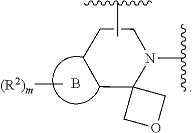

In some embodiments, Ring A is

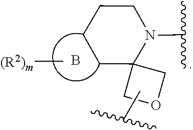

In some embodiments, Ring A is

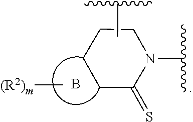

In some embodiments, Ring A is

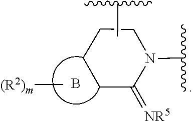

In some embodiments, Ring A is

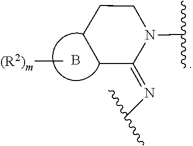

In some embodiments, Ring A is

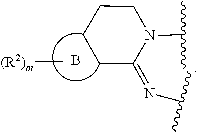

In some embodiments, Ring A is

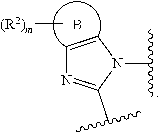

In some embodiments, Ring A is

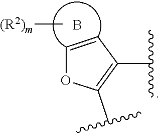

In some embodiments, Ring A is

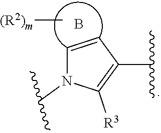

In some embodiments, Ring A is

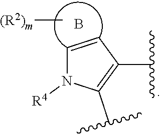

In some embodiments, Ring A is

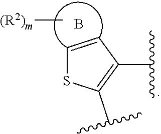

In some embodiments, Ring A is selected from those depicted in Table 1 below.

As defined above and described herein, Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;

In some embodiments, Ring B is a fused 6-membered aryl. In some embodiments, Ring B is a fused 6-membered heteroaryl containing 1-4 heteroatoms independently selected from nitrogen, oxygen, or sulfur. In some embodiments, Ring B is a fused 5 to 7-membered saturated or partially unsaturated carbocyclyl. In some embodiments, Ring B is fused 5 to 7-membered saturated or partially saturated heterocyclyl with 1-3 heteroatoms independently selected from boron, nitrogen, oxygen, silicon, or sulfur. In some embodiments, Ring B is fused 5-membered heteroaryl with 1-4 heteroatoms independently selected from boron, nitrogen, oxygen, silicon, or sulfur.

In some embodiments, Ring B is

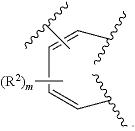

In some embodiments, Ring B is

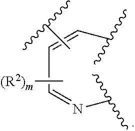

In some embodiments, Ring B is

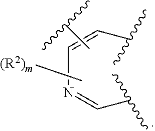

In some embodiments, Ring B is selected from those depicted in Table 1 below.

As defined above and described herein, Ring C is a mono- or bicyclic ring selected from

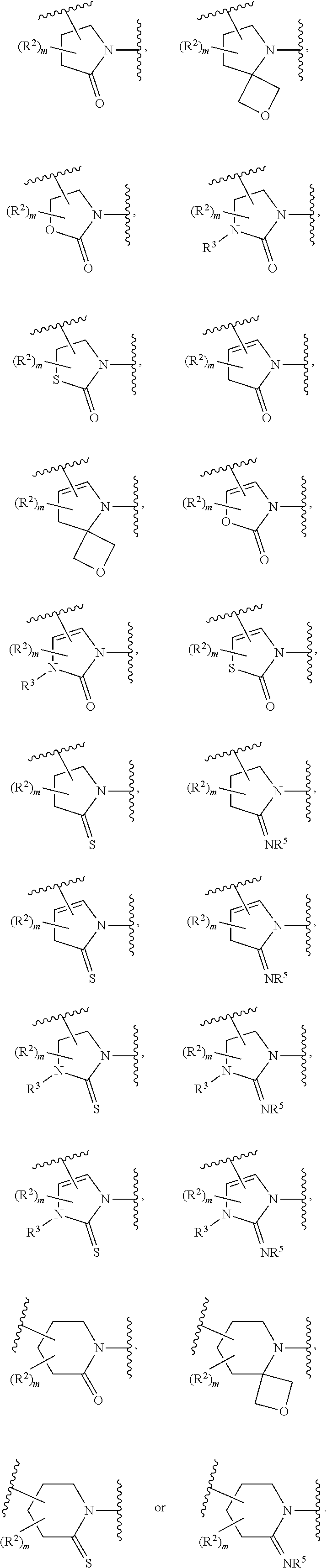

In some embodiments, Ring C is

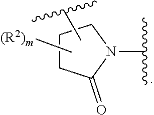

In some embodiments, Ring C is

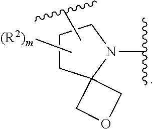

In some embodiments, Ring C is

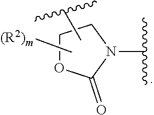

In some embodiments, Ring C is

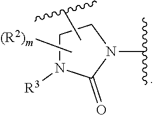

In some embodiments, Ring C is

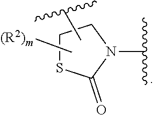

In some embodiments, Ring C is

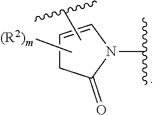

In some embodiments, Ring C is

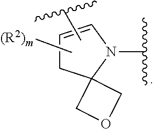

In some embodiments, Ring C is

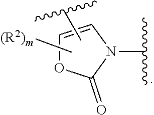

In some embodiments, Ring C is

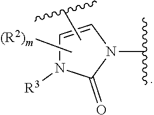

In some embodiments, Ring C is

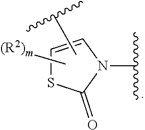

In some embodiments, Ring C is

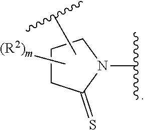

In some embodiments, Ring C is

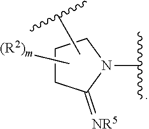

In some embodiments, Ring C is

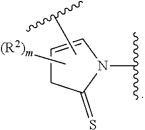

In some embodiments, Ring C is

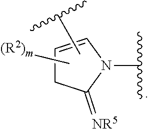

In some embodiments, Ring C is

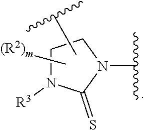

In some embodiments, Ring C is

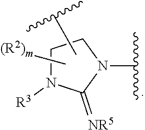

In some embodiments, Ring C is

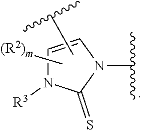

In some embodiments, Ring C is

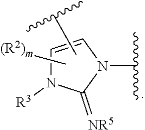

In some embodiments, Ring C is

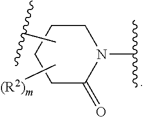

In some embodiments, Ring C is

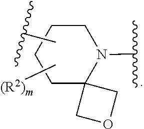

In some embodiments, Ring C is

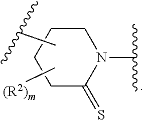

In some embodiments, Ring C is

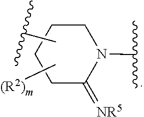

In some embodiments, Ring C is

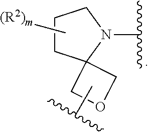

In some embodiments, Ring C is

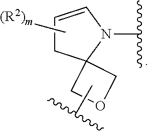

In some embodiments, Ring C is

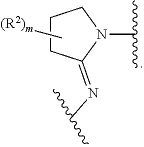

In some embodiments, Ring C is

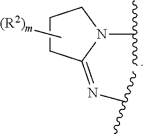

In some embodiments, Ring C is

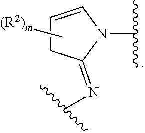

In some embodiments, Ring C is

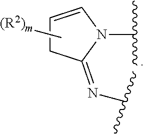

In some embodiments, Ring C is

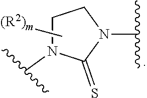

In some embodiments, Ring C is

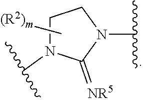

In some embodiments, Ring C is

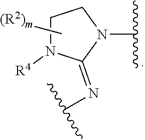

In some embodiments, Ring C is

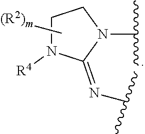

In some embodiments, Ring C is

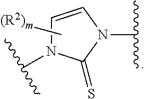

In some embodiments, Ring C is

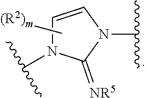

In some embodiments, Ring C is

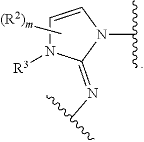

In some embodiments, Ring C is

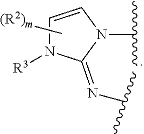

In some embodiments, Ring C is

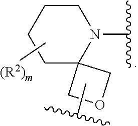

In some embodiments, Ring C is

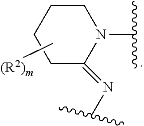

In some embodiments, Ring C is

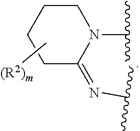

In some embodiments, Ring C is a mono- or bicyclic ring selected from

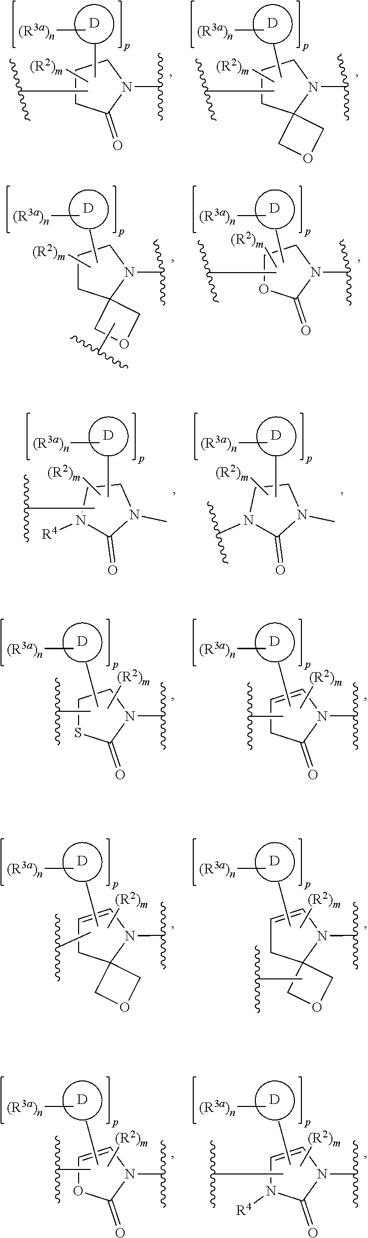

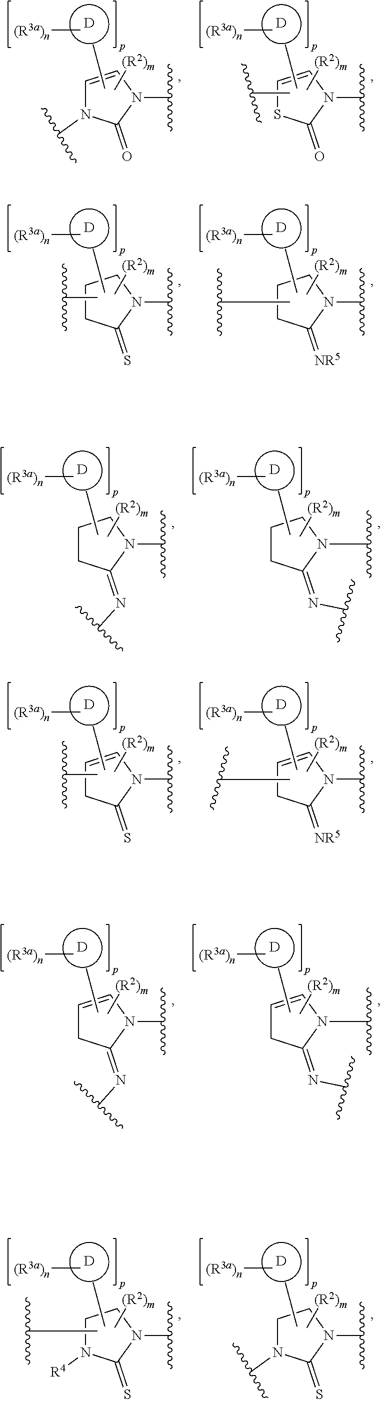

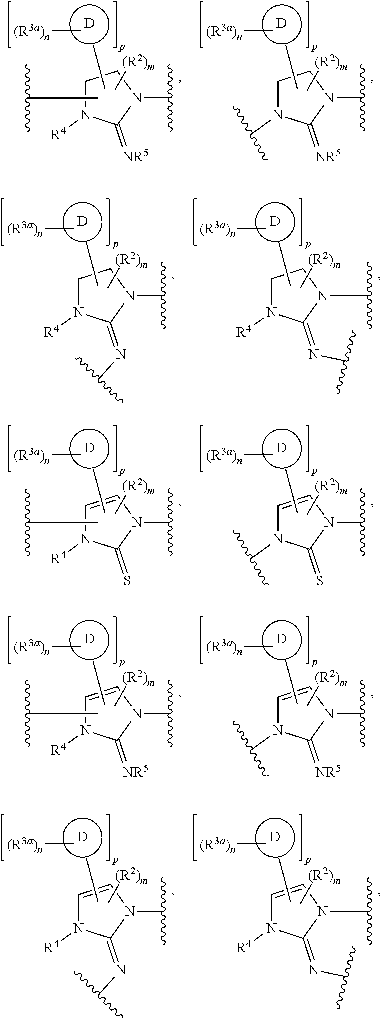

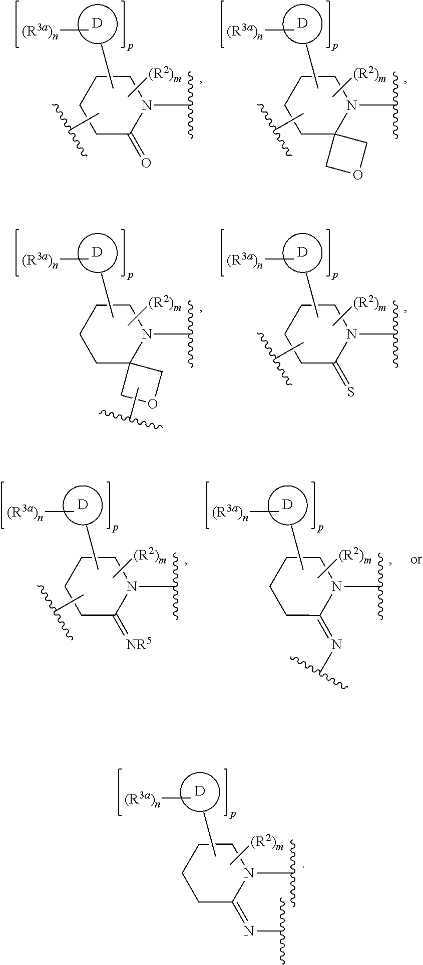

In some embodiments, Ring C is selected from those depicted in Table 1 below.

As defined above and described herein, Ring D is a ring selected from 6 to 10-membered aryl or heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;

In some embodiments, Ring D is a 6 to 10-membered aryl. In some embodiments, Ring D is a 6 to 10-membered heteroaryl containing 1-4 heteroatoms independently selected from nitrogen, oxygen, or sulfur. In some embodiments, Ring D is a 5 to 7-membered saturated or partially unsaturated carbocyclyl. In some embodiments, Ring D is 5 to 7-membered saturated or partially saturated heterocyclyl with 1-3 heteroatoms independently selected from boron, nitrogen, oxygen, silicon, or sulfur. In some embodiments, Ring D is 5-membered heteroaryl with 1-4 heteroatoms independently selected from boron, nitrogen, oxygen, silicon, or sulfur. In some embodiments, Ring D in phenyl. In some embodiments, Ring D is pyridinyl.

In some embodiments, Ring D is selected from those depicted in Table 1 below.

As defined above and described herein, each of Ring E, Ring F, and Ring G is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur, wherein Ring E, Ring F, and Ring G is independently and optionally substituted with 1-2 oxo groups.

In some embodiments, each of Ring E, Ring F, and Ring G is independently a fused ring selected from 6-membered aryl. In some embodiments, each of Ring E, Ring F, and Ring G is independently a fused ring selected from 6-membered heteroaryl containing 1-4 heteroatoms independently selected from nitrogen, oxygen, or sulfur. In some embodiments, each of Ring E, Ring F, and Ring G is independently a fused ring selected from a 5 to 7-membered saturated or partially unsaturated carbocyclyl. In some embodiments, each of Ring E, Ring F, and Ring G is independently a fused ring selected from a 5 to 7-membered saturated or partially unsaturated heterocyclyl with 1-3 heteroatoms independently selected from boron, nitrogen, oxygen, silicon, or sulfur. In some embodiments, each of Ring E, Ring F, and Ring G is independently a fused ring selected from a 5-membered heteroaryl with 1-4 heteroatoms independently selected from nitrogen, oxygen or sulfur. In some embodiments, Ring E, Ring F, and Ring G is independently and optionally substituted with 1-2 oxo groups.

In some embodiments, Ring F is

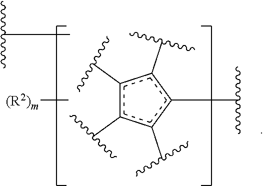

In some embodiments, each of Ring E and Ring G is independently

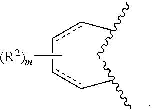

In some embodiments, each of Ring E and Ring G is independently

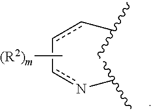

In some embodiments, each of Ring E and Ring G is independently

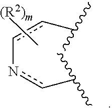

In some embodiments, each of Ring E and Ring G is independently

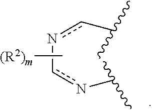

In some embodiments, each of Ring E and Ring G is independently

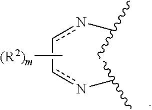

In some embodiments, Ring E, Ring F, and Ring G is

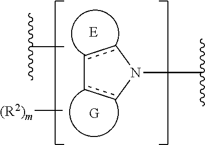

In some embodiments, Ring E, Ring F, and Ring G is

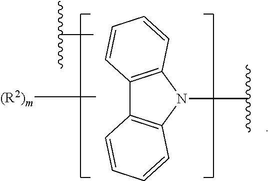

In some embodiments, Ring E, Ring F, and Ring G is

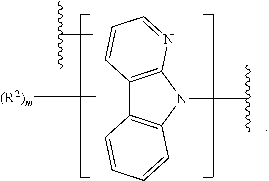

In some embodiments, Ring E, Ring F, and Ring G is selected from those depicted in Table 1, below.

As defined above and described herein, Ring H is a ring selected from a 7-9 membered saturated or partially unsaturated carbocyclyl or heterocyclyl ring with 1-3 heteroatoms independently selected from boron, nitrogen, oxygen, silicon, or sulfur, wherein Ring E is optionally further substituted with 1-2 oxo groups.

In some embodiments, Ring H is a ring selected from a 7-9 membered saturated or partially unsaturated carbocyclyl or heterocyclyl ring with 1-3 heteroatoms independently selected from boron, nitrogen, oxygen, silicon, or sulfur, wherein Ring H is optionally further substituted with 1-2 oxo groups.

As defined above and described herein, each of Ring I and Ring J is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur

In some embodiments, each of Ring I and Ring J is independently a 6-membered aryl. In some embodiments, each of Ring I and Ring J is independently a 6-membered heteroaryl containing 1-4 heteroatoms independently selected from nitrogen, oxygen, or sulfur. In some embodiments, each of Ring I and Ring J is independently a 5 to 7-membered saturated or partially unsaturated carbocyclyl. In some embodiments, each of Ring I and Ring J is independently a 5 to 7-membered saturated or partially unsaturated heterocyclyl with 1-3 heteroatoms independently selected from boron, nitrogen, oxygen, silicon, or sulfur. In some embodiments, each of Ring I and Ring J is independently a 5-membered heteroaryl with 1-3 heteroatoms independently selected from nitrogen, oxygen or sulfur.

In some embodiments, Ring I and Ring J is selected from those depicted in Table 1, below.

As defined above and described herein, Ring K is a fused ring selected from a 7-12 membered saturated or partially unsaturated carbocyclyl or heterocyclyl with 1-3 heteroatoms independently selected from boron, nitrogen, oxygen, silicon, or sulfur, wherein Ring H is optionally further substituted with 1-2 oxo groups.

In some embodiments, Ring K is a fused ring selected from a 7-12 membered saturated or partially unsaturated carbocyclyl. In some embodiments, Ring K is a 7-12 membered saturated or partially unsaturated heterocyclyl ring with 1-3 heteroatoms independently selected from boron, nitrogen, oxygen, silicon, or sulfur. In some embodiments, Ring K is optionally further substituted with 1-2 oxo groups.

In some embodiments, Ring K is selected from those depicted in Table 1 below.

As defined above and described herein, Ring M is selected from

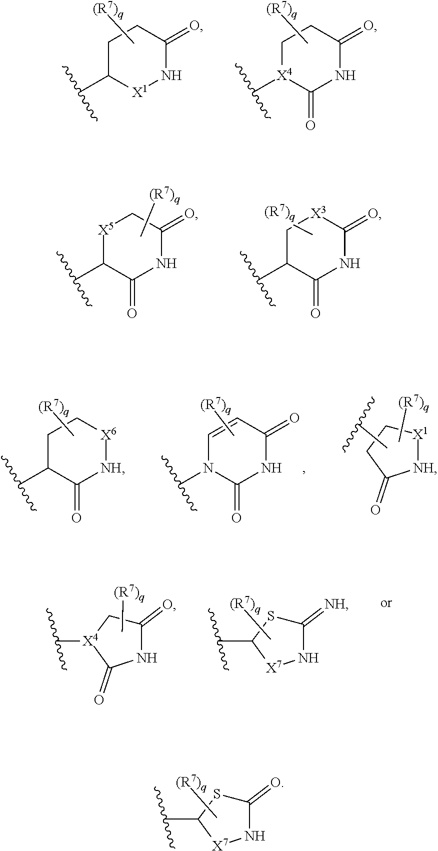

In some embodiments, Ring M is

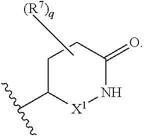

In some embodiments, Ring M is

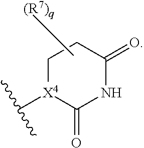

In some embodiments, Ring M is

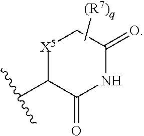

In some embodiments, Ring M is

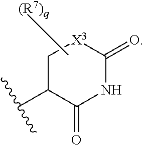

In some embodiments, Ring M is

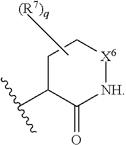

In some embodiments, Ring M is

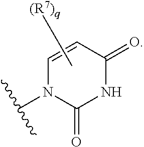

In some embodiments, Ring M is

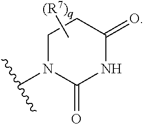

In some embodiments, Ring M is

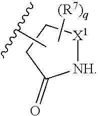

In some embodiments, Ring M is

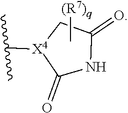

In some embodiments, Ring M is

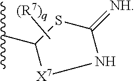

In some embodiments, Ring M is

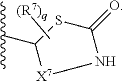

In some embodiments, Ring M is selected from those depicted in Table 1 below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;

In some embodiments, L¹ is a covalent bond. In some embodiments, L¹ is a C₁₋₃ aliphatic. In some embodiments, L¹ is —CH₂—. In some embodiments, L¹ is —C(D)(H)—. In some embodiments, L¹ is —C(D)₂-. In some embodiments, L¹ is —CH₂CH₂—. In some embodiments, L¹ is —NR—. In some embodiments, L¹ is —CH₂NR—. In some embodiments, L¹ is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—.

In some embodiments, Ring L¹ is selected from those depicted in Table 1 below.

As defined above and described herein, [symbol] is a single or double bond.

In some embodiments, [symbol] is a single bond. In some embodiments, [symbol] is a double bond.

In some embodiments, [symbol] is selected from those depicted in Table 1 below.

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1 below.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In some embodiments, n is selected from those depicted in Table 1 below.

As defined above and described herein, p is 0 or 1.

In some embodiments, p is 0. In some embodiments, p is 1.

In some embodiments, p is selected from those depicted in Table 1 below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiments, LBM is

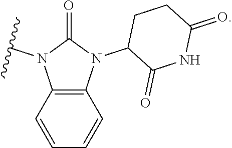 [SMILES: *n1c(=O)n(C2CCC(=O)NC2=O)c2ccccc21]

In some embodiments, LBM is

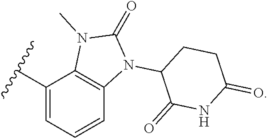 [SMILES: *c1cccc2c1n(C)c(=O)n2C1CCC(=O)NC1=O]

In some embodiments, LBM is

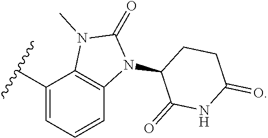 [SMILES: *c1cccc2c1n(C)c(=O)n2[C@H]1CCC(=O)NC1=O]

In some embodiments, LBM is

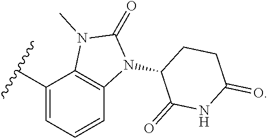 [SMILES: *c1cccc2c1n(C)c(=O)n2[C@@H]1CCC(=O)NC1=O]

In some embodiments, LBM is

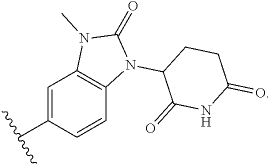 [SMILES: *c1ccc2c(c1)n(C)c(=O)n2C1CCC(=O)NC1=O]

In some embodiments, LBM is

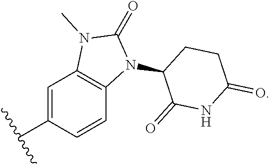 [SMILES: *c1ccc2c(c1)n(C)c(=O)n2[C@H]1CCC(=O)NC1=O]

In some embodiments, LBM is

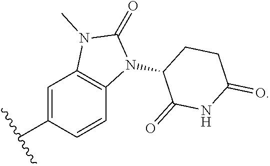 [SMILES: *c1ccc2c(c1)n(C)c(=O)n2[C@@H]1CCC(=O)NC1=O]

In some embodiments, LBM is

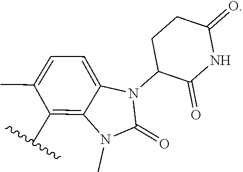 [SMILES: *c1c(C)ccc2c1n(C)c(=O)n2C1CCC(=O)NC1=O]

In some embodiments, LBM is

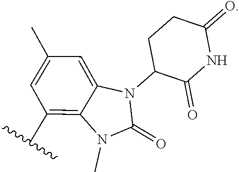 [SMILES: *c1cc(C)cc2c1n(C)c(=O)n2C1CCC(=O)NC1=O]

In some embodiments, LBM is

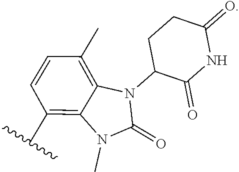 [SMILES: *c1ccc(C)c2c1n(C)c(=O)n2C1CCC(=O)NC1=O]

In some embodiments, LBM is

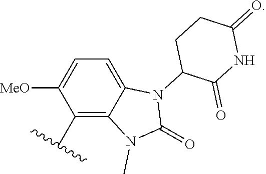 [SMILES: *c1c(OC)ccc2c1n(C)c(=O)n2C1CCC(=O)NC1=O]

In some embodiments, LBM is

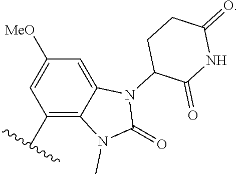 [SMILES: *c1cc(OC)cc2c1n(C)c(=O)n2C1CCC(=O)NC1=O]

In some embodiments, LBM is

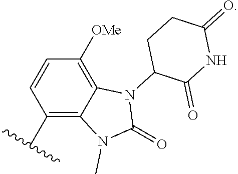 [SMILES: *c1ccc(OC)c2c1n(C)c(=O)n2C1CCC(=O)NC1=O]

In some embodiments, LBM is

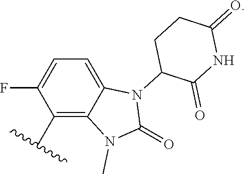 [SMILES: *c1c(F)ccc2c1n(C)c(=O)n2C1CCC(=O)NC1=O]

In some embodiments, LBM is

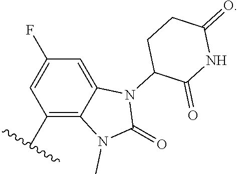 [SMILES: *c1cc(F)cc2c1n(C)c(=O)n2C1CCC(=O)NC1=O]

In some embodiments, LBM is

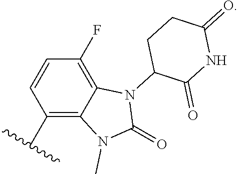 [SMILES: *c1ccc(F)c2c1n(C)c(=O)n2C1CCC(=O)NC1=O]

In some embodiments, LBM is

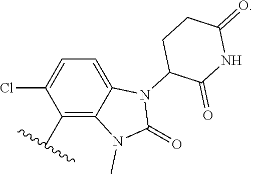 [SMILES: *c1c(Cl)ccc2c1n(C)c(=O)n2C1CCC(=O)NC1=O]

In some embodiments, LBM is

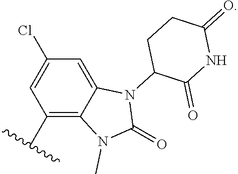 [SMILES: *c1cc(Cl)cc2c1n(C)c(=O)n2C1CCC(=O)NC1=O]

In some embodiments, LBM is

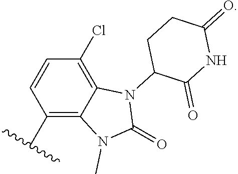 [SMILES: *c1ccc(Cl)c2c1n(C)c(=O)n2C1CCC(=O)NC1=O]

In some embodiments, LBM

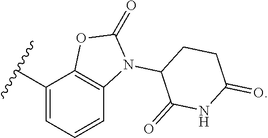 [SMILES: *c1cccc2c1oc(=O)n2C1CCC(=O)NC1=O]

In some embodiments, LBM is

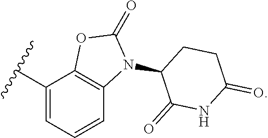 [SMILES: *c1cccc2c1oc(=O)n2[C@H]1CCC(=O)NC1=O]

In some embodiments, LBM is

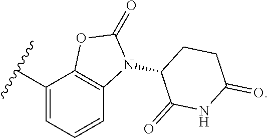 [SMILES: *c1cccc2c1oc(=O)n2[C@@H]1CCC(=O)NC1=O]

In some embodiments, LBM is

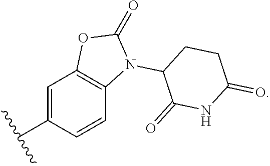 [SMILES: *c1ccc2c(c1)oc(=O)n2C1CCC(=O)NC1=O]

In some embodiments, LBM is

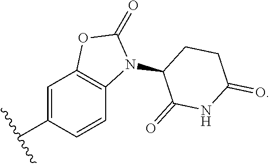 [SMILES: *c1ccc2c(c1)oc(=O)n2[C@H]1CCC(=O)NC1=O]

In some embodiments, LBM is

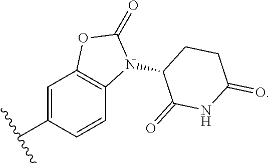 [SMILES: *c1ccc2c(c1)oc(=O)n2[C@@H]1CCC(=O)NC1=O]

In some embodiments, LBM is

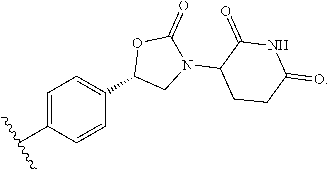 [SMILES: *c1ccc([C@H]2CN(C3CCC(=O)NC3=O)C(=O)O2)cc1]

In some embodiments, LBM is

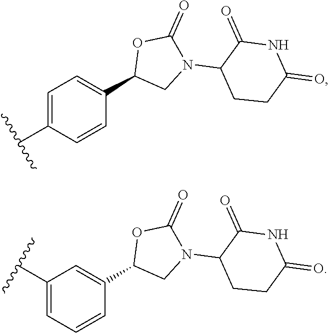 [SMILES: *c1ccc([C@@H]2CN(C3CCC(=O)NC3=O)C(=O)O2)cc1 ; *c1cccc([C@H]2CN(C3CCC(=O)NC3=O)C(=O)O2)c1]

In some embodiments, LBM is

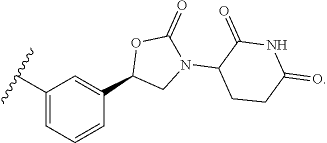 [SMILES: *c1cccc([C@@H]2CN(C3CCC(=O)NC3=O)C(=O)O2)c1]

In some embodiments, LBM is

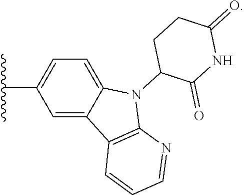 [SMILES: *c1ccc2c(c1)c1cccnc1n2C1CCC(=O)NC1=O]

In some embodiments, LBM is

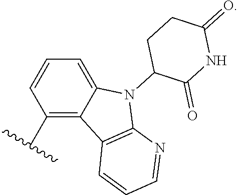 [SMILES: *c1cccc2c1c1cccnc1n2C1CCC(=O)NC1=O]

In some embodiments, LBM is

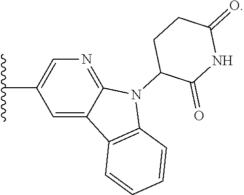 [SMILES: *c1cnc2c(c1)c1ccccc1n2C1CCC(=O)NC1=O]

In some embodiments, LBM is

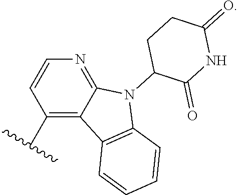 [SMILES: *c1ccnc2c1c1ccccc1n2C1CCC(=O)NC1=O]

In some embodiments, LBM is

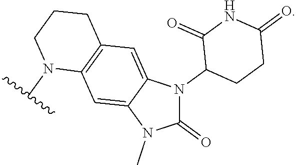 [SMILES: *N1CCCc2cc3c(cc21)n(C)c(=O)n3C1CCC(=O)NC1=O]

In some embodiments, LBM is

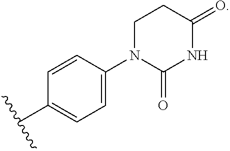 [SMILES: *c1ccc(N2CCC(=O)NC2=O)cc1]

In some embodiments, LBM is

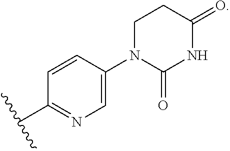 [SMILES: *c1ccc(N2CCC(=O)NC2=O)cn1]

In some embodiments, LBM is

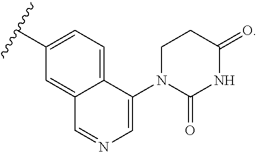 [SMILES: *c1ccc2c(N3CCC(=O)NC3=O)cncc2c1]

In some embodiments, LBM is

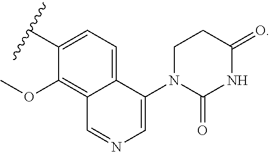 [SMILES: *c1ccc2c(N3CCC(=O)NC3=O)cncc2c1OC]

In some embodiments, LBM is

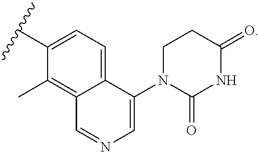 [SMILES: *c1ccc2c(N3CCC(=O)NC3=O)cncc2c1C]

In some embodiments, LBM is

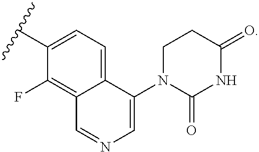 [SMILES: *c1ccc2c(N3CCC(=O)NC3=O)cncc2c1F]

In some embodiments, LBM is

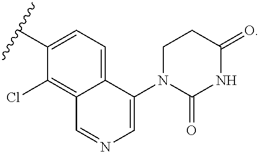 [SMILES: *c1ccc2c(N3CCC(=O)NC3=O)cncc2c1Cl]

In some embodiments, LBM is

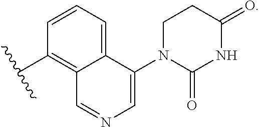 [SMILES: *c1cccc2c(N3CCC(=O)NC3=O)cncc12]

In some embodiments, LBM is selected from those in Table 1 below.

In some embodiments, the present invention provides the compound of formula I-a, wherein LBM is

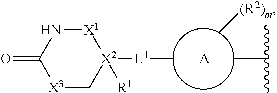

Ring Q and Ring P form an indazole ring, and X is cyclohexyl as shown, to provide a compound of formula I-e-1:

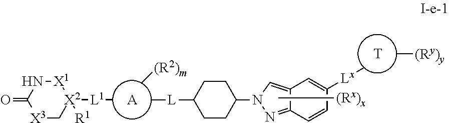

or a pharmaceutically acceptable salt thereof, wherein each of X¹, X², X³, R¹, R², L¹, Ring A, and m of the LBM, L, and L^{x}, Ring T, R^{x}, R^{y}, x, and y of the IRAK moiety is as defined above and described in embodiments herein, both singly and in combination.

In some embodiments, the present invention provides the compound of formula I-a, wherein LBM is

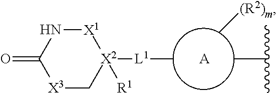

Ring Q and Ring P form a 6-azaindazole ring, and X is cyclohexyl as shown, to provide a compound of formula I-e-2:

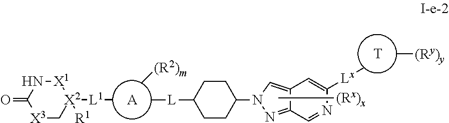

or a pharmaceutically acceptable salt thereof, wherein each of X¹, X², X³, R¹, R², L¹, Ring A, and m of the LBM, L, and L^{x}, Ring T, R^{x}, R^{y}, x, and y of the IRAK moiety is as defined above and described in embodiments herein, both singly and in combination.

In some embodiments, the present invention provides the compound of formula I-a, wherein LBM is

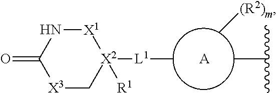

m is 1 and R² is —OC₁₋₆alkyl, x is 0, Ring Q and Ring P form an indazole ring, and X is cyclohexyl as shown, to provide a compound of formula I-f-1:

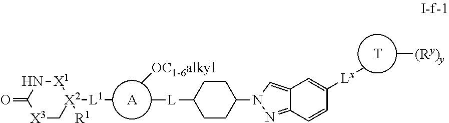

or a pharmaceutically acceptable salt thereof, wherein each of X¹, X², X³, R¹, R², L, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

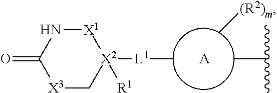

m is 1 and R² is —OMe, x is 0, Ring Q and Ring P form an indazole ring, and X is cyclohexyl as shown, to provide a compound of formula I-f-2:

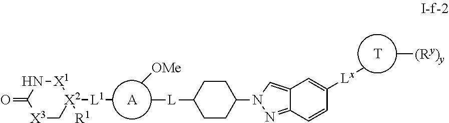

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

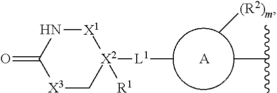

### L is

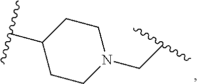 [SMILES: *CN1CCC(*)CC1]

x is 0, Ring Q and Ring P form an indazole ring, and X is cyclohexyl as shown, to provide a compound of formula I-f-3:

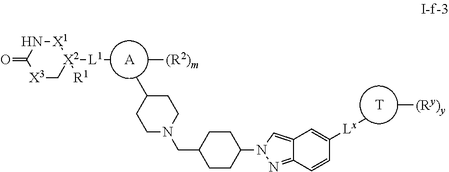

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

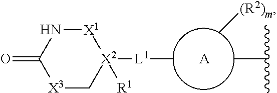

m is 1 and R² is —OC₁₋₆alkyl, L is

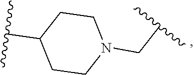 [SMILES: *CN1CCC(*)CC1]

x is 0, Ring Q and Ring P form an indazole ring, and X is cyclohexyl as shown, to provide a compound of formula I-f-4:

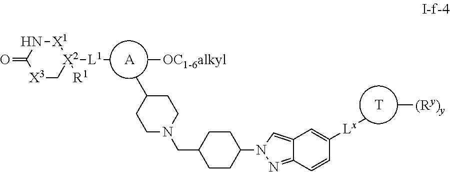

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

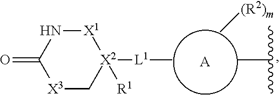

### m is 1 and R² is —OMe, L is

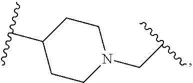 [SMILES: *CN1CCC(*)CC1]

x is 0, Ring Q and Ring P form an indazole ring, and X is cyclohexyl as shown, to provide a compound of formula I-f-5:

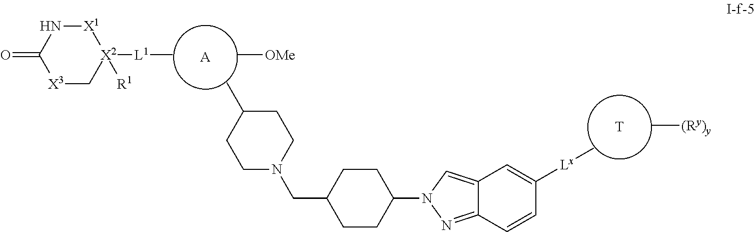

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

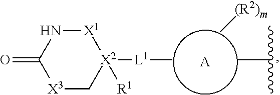

m is 1 and R² is —OC₁₋₆alkyl, x is 0, Ring Q and Ring P form a 6-azaindazole ring, and X is cyclohexyl as shown, to provide a compound of formula I-f-6:

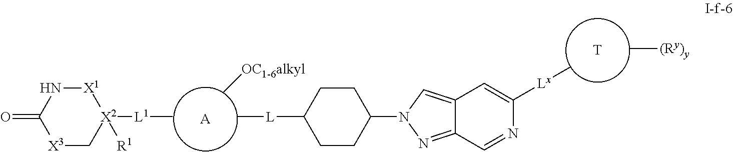

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

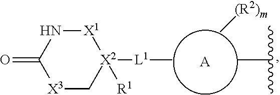

m is 1 and R² is —OMe, x is 0, Ring Q and Ring P form a 6-azaindazole ring, and X is cyclohexyl as shown, to provide a compound of formula I-f-7:

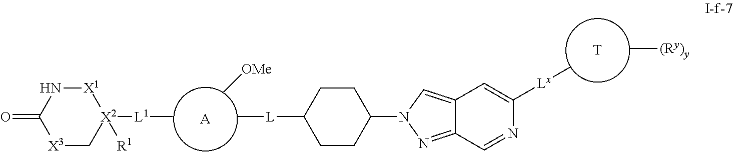

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

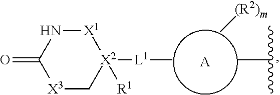

### L is

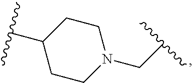 [SMILES: *CN1CCC(*)CC1]

x is 0, Ring Q and Ring P form a 6-azaindazole ring, and X is cyclohexyl as shown, to provide a compound of formula I-f-8:

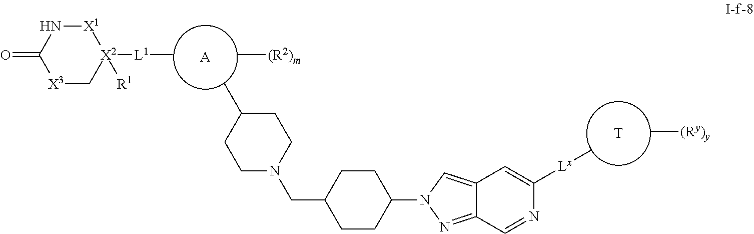

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

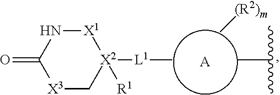

m is 1 and R² is —OC₁₋₆alkyl, L is

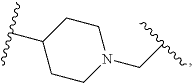 [SMILES: *CN1CCC(*)CC1]

x is 0, Ring Q and Ring P form a 6-azaindazole ring, and X is cyclohexyl as shown, to provide a compound of formula I-f-9:

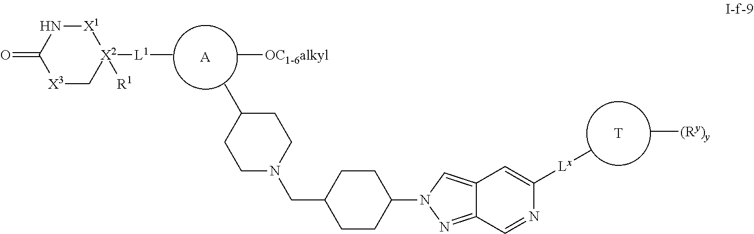

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

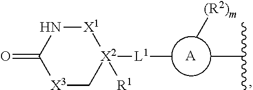

### m is 1 and R² is —OMe, L is

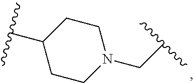 [SMILES: *CN1CCC(*)CC1]

x is 0, Ring Q and Ring P form a 6-azaindazole ring, and X is cyclohexyl as shown, to provide a compound of formula I-f-10:

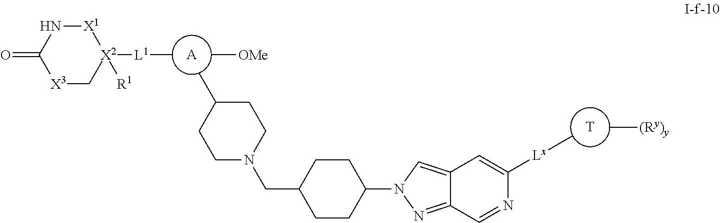

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

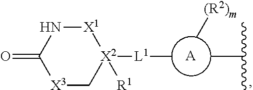

x is 1 and R^{x} is methyl, Ring Q and Ring P form an indazole ring, and X is cyclohexyl as shown, to provide a compound of formula I-f-11:

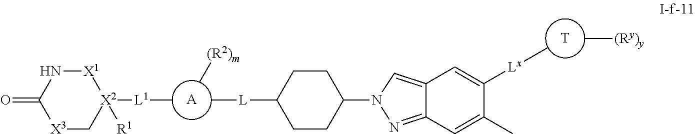

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

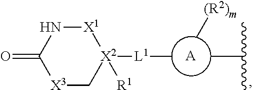

### L is

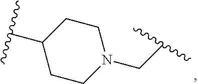 [SMILES: *CN1CCC(*)CC1]

x is 1 and R^{x} is methyl, Ring Q and Ring P form an indazole ring, and X is cyclohexyl as shown, to provide a compound of formula I-f-12:

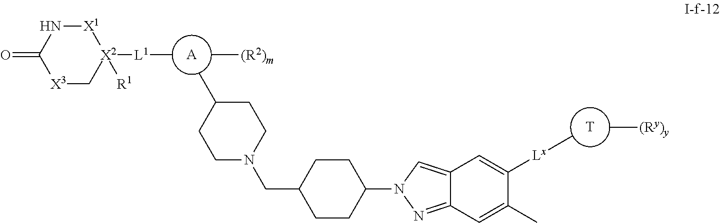

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

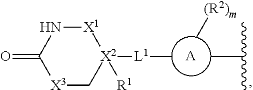

### L is

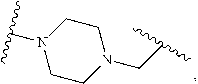 [SMILES: *CN1CCN(*)CC1]

x is 1 and R^{x} is methyl, Ring Q and Ring P form an indazole ring, and X is cyclohexyl as shown, to provide a compound of formula I-f-13:

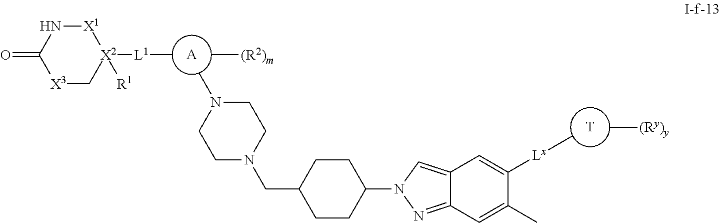

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

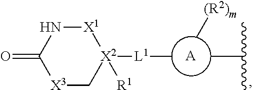

m is 1 and R² is —C₁₋₆alkyl, x is 0, Ring Q and Ring P form a 6-azaindazole ring, and X is cyclohexyl as shown, to provide a compound of formula I-f-14:

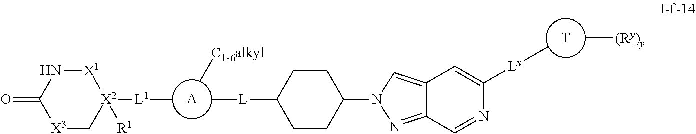

or a pharmaceutically acceptable salt thereof, wherein each of X¹, X², X³, R¹, R², LV, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

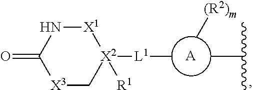

m is 1 and R² is methyl, x is 0, Ring Q and Ring P form a 6-azaindazole ring, and X is cyclohexyl as shown, to provide a compound of formula I-f-15:

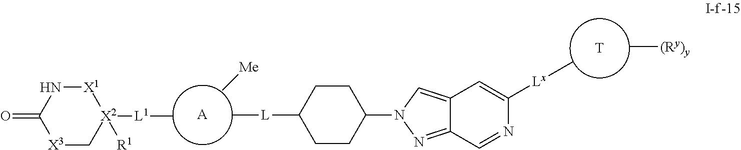

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

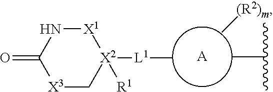

m is 1 and R² is —C₁₋₆alkyl, L is

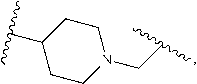 [SMILES: *CN1CCC(*)CC1]

x is 0, Ring Q and Ring P form a 6-azaindazole ring, and X is cyclohexyl as shown, to provide a compound of formula I-f-16:

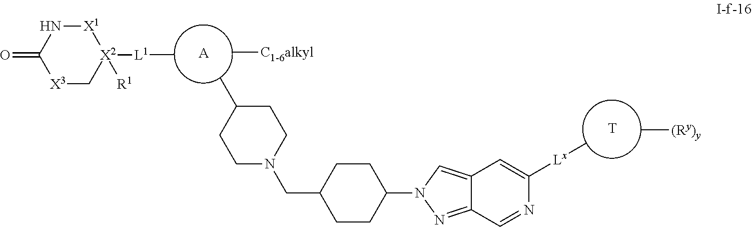

or a pharmaceutically acceptable salt thereof, wherein each of X¹, X², X³, R¹, R², L, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

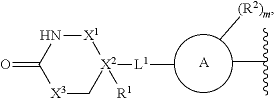

m is 1 and R² is methyl, L is

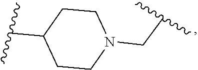 [SMILES: *CN1CCC(*)CC1]

x is 0, Ring Q and Ring P form a 6-azaindazole ring, and X is cyclohexyl as shown, to provide a compound of formula I-f-17:

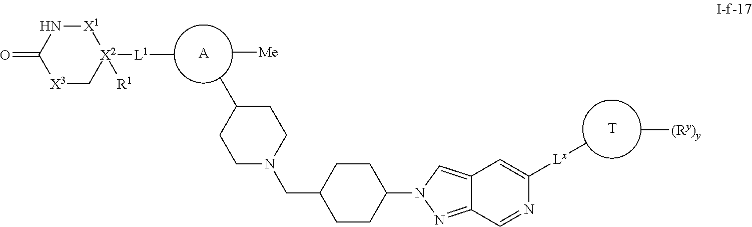

or a pharmaceutically acceptable salt thereof, wherein each of X¹, X², X³, R¹, R², L, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

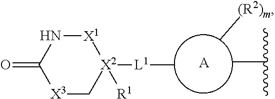

m is 1 and R² is halo, x is 0, Ring Q and Ring P form a 6-azaindazole ring, and X is cyclohexyl as shown, to provide a compound of formula I-f-18:

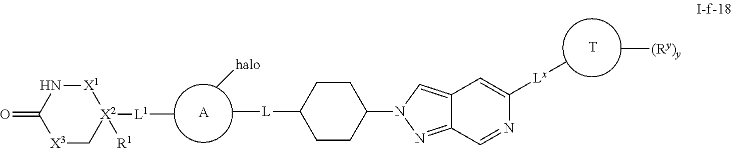

or a pharmaceutically acceptable salt thereof, wherein each of X¹, X², X³, R¹, R², L, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

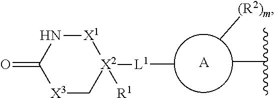

m is 1 and R² is fluoro, x is 0, Ring Q and Ring P form a 6-azaindazole ring, and X is cyclohexyl as shown, to provide a compound of formula I-f-19:

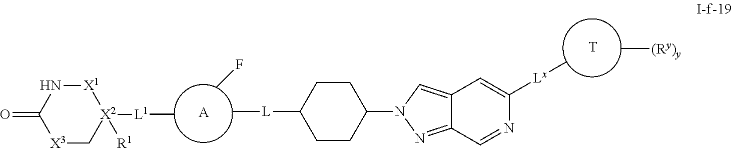

or a pharmaceutically acceptable salt thereof, wherein each of X¹, X², X³, R¹, R², L, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

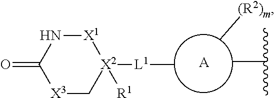

m is 1 and R² is halo, L is

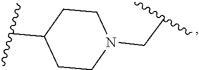 [SMILES: *CN1CCC(*)CC1]

x is 0, Ring Q and Ring P form a 6-azaindazole ring, and X is cyclohexyl as shown, to provide a compound of formula I-f-20:

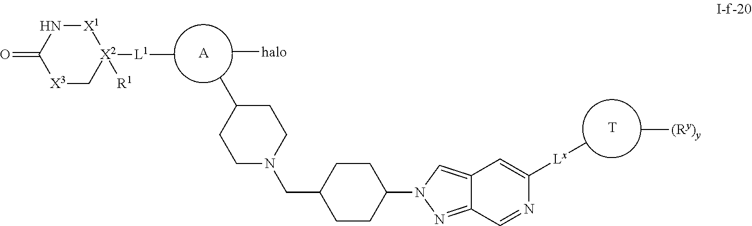

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

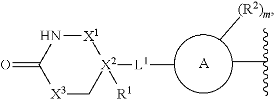

m is 1 and R² is fluoro, L is

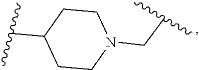 [SMILES: *CN1CCC(*)CC1]

x is 0, Ring Q and Ring P form a 6-azaindazole ring, and X is cyclohexyl as shown, to provide a compound of formula I-f-21:

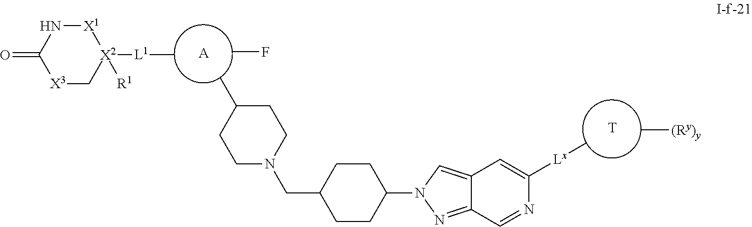

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

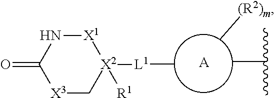

x is 1 and R^{x} is chloro, Ring Q and Ring P form an indazole ring, and X is cyclohexyl as shown, to provide a compound of formula I-f-22:

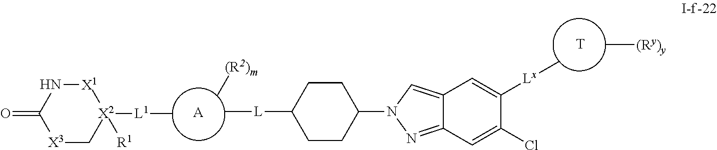

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

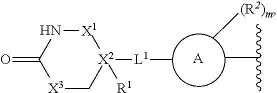

### L is

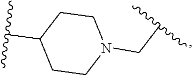 [SMILES: *CN1CCC(*)CC1]

x is 1 and R^{x} is chloro, Ring Q and Ring P form an indazole ring, and X is cyclohexyl as shown, to provide a compound of formula I-f-23:

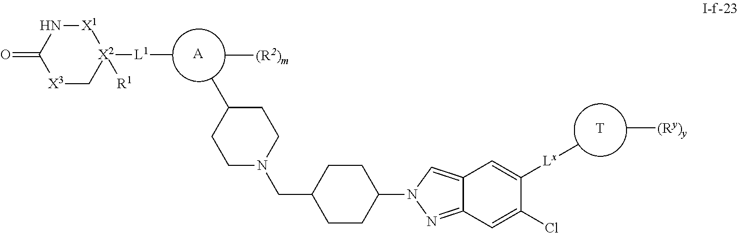

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

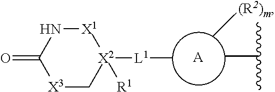

x is 1 and R^{x} is —OMe, Ring Q and Ring P form an indazole ring, and X is cyclohexyl as shown, to provide a compound of formula I-f-24:

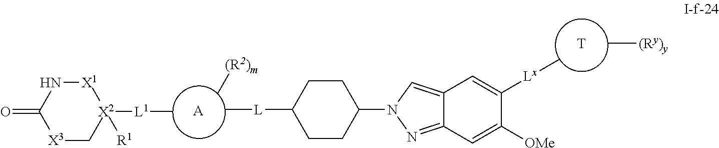

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

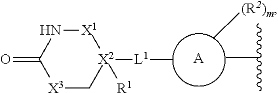

### L is

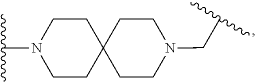 [SMILES: *CN1CCC2(CCN(*)CC2)CC1]

x is 1 and R^{x} is —OMe, Ring Q and Ring P form an indazole ring, and X is cyclohexyl as shown, to provide a compound of formula I-f-25:

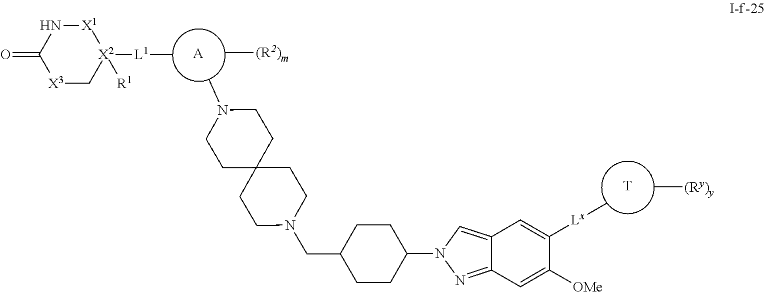

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

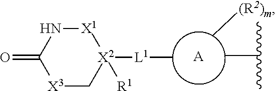

x is 1 and R^{x} is —OMe, Ring Q and Ring P form an indazole ring, and X is cyclohexyl as shown, to provide a compound of formula I-f-26:

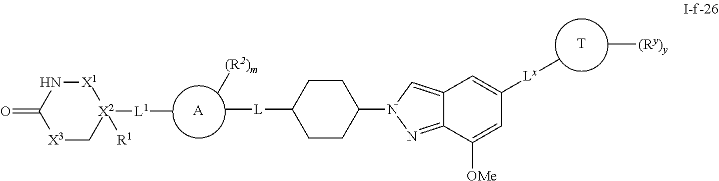

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

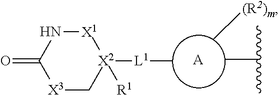

### L is

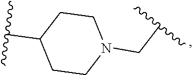 [SMILES: *CN1CCC(*)CC1]

x is 1 and R is —OMe, Ring Q and Ring P form an indazole ring, and X is cyclohexyl as shown, to provide a compound of formula I-f-27:

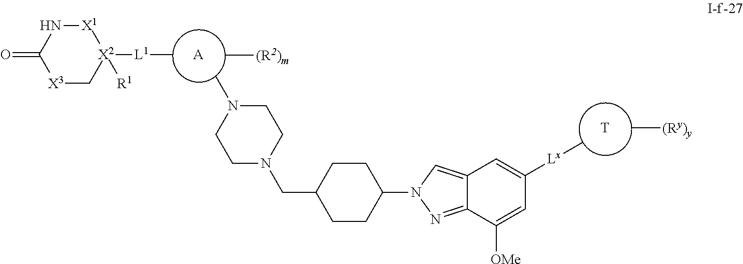

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

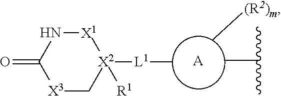

x is 1 and R^{x} is —S(O)Me, Ring Q and Ring P form an indazole ring, and X is cyclohexyl as shown, to provide a compound of formula I-f-28:

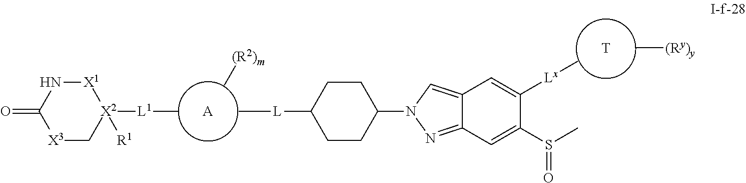

or a pharmaceutically acceptable salt thereof, wherein each of X¹, X², X³, R¹, R², L, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

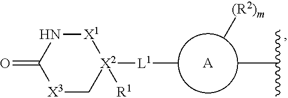

### L is

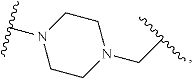 [SMILES: *CN1CCN(*)CC1]

x is 1 and R is —S(O)Me, Ring Q and Ring P form an indazole ring, and X is cyclohexyl as shown, to provide a compound of formula I-f-29:

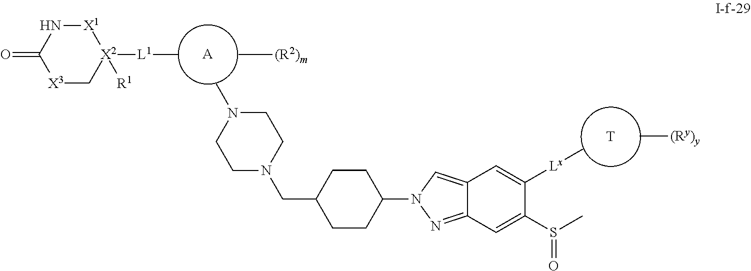

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

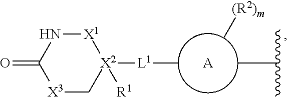

m is 1 and R² is halo, x is 1 and R^{x} is —OMe, Ring Q and Ring P form a 6-azaindazole ring, and X is cyclobutyl as shown, to provide a compound of formula I-f-30:

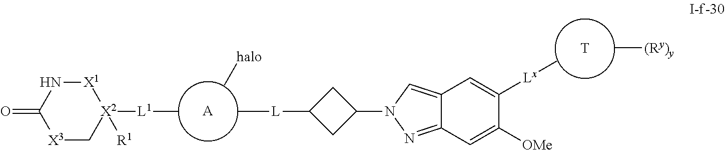

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

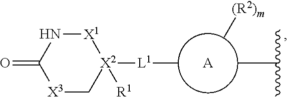

m is 1 and R² is fluoro, x is 1 and R is —OMe, Ring Q and Ring P form a 6-azaindazole ring, and X is cyclobutyl as shown, to provide a compound of formula I-f-31:

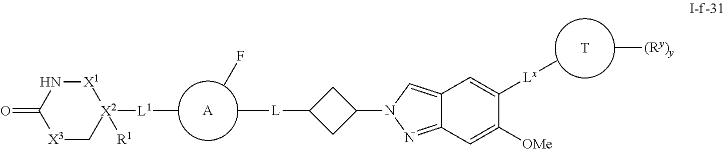

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

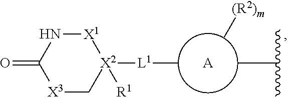

m is 1 and R² is halo, L is

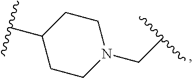 [SMILES: *CN1CCC(*)CC1]

x is 1 and R^{x} is —OMe, Ring Q and Ring P form a 6-azaindazole ring, and X is cyclobutyl as shown, to provide a compound of formula I-f-32:

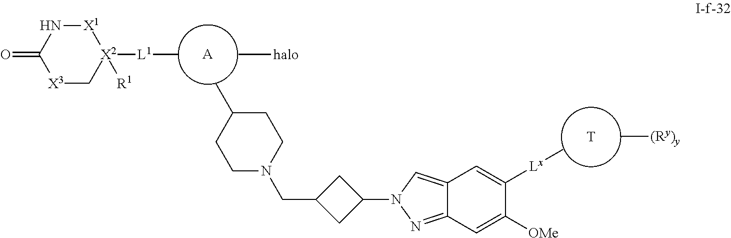

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

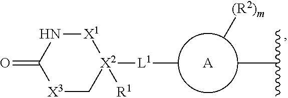

m is 1 and R² is fluoro, L is

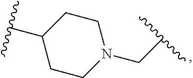 [SMILES: *CN1CCC(*)CC1]

x is 1 and R is —OMe, Ring Q and Ring P form a 6-azaindazole ring, and X is cyclobutyl as shown, to provide a compound of formula I-f-33:

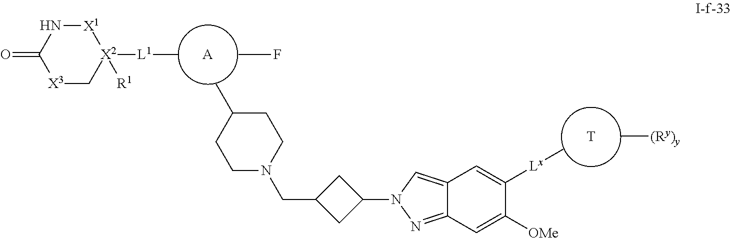

or a pharmaceutically acceptable salt thereof, wherein each of X¹, X², X³, R¹, R², L¹, and Ring A of the LBM, and L^{x}, Ring T, R^{y}, and y of the IRAK moiety is as defined above and described in embodiments herein, both singly and in combination.

In some embodiments, the present invention provides the compound of formula I-a, wherein LBM is

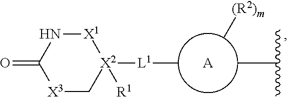

x is 1 and R^{x} is —SO₂Me, Ring Q and Ring P form an indazole ring, and X is cyclohexyl as shown, to provide a compound of formula I-f-34:

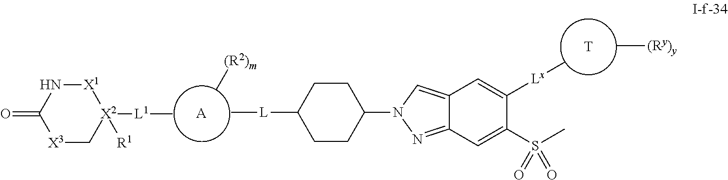

or a pharmaceutically acceptable salt thereof, wherein each of X¹, X², X³, R¹, R², L¹, and Ring A of the LBM, L, and L^{x}, Ring T, R^{y}, and y of the IRAK moiety is as defined above and described in embodiments herein, both singly and in combination.

In some embodiments, the present invention provides the compound of formula I-a, wherein LBM is

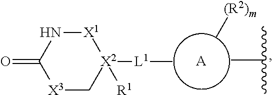

### L is

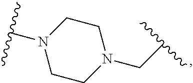 [SMILES: *CN1CCN(*)CC1]

x is 1 and R^{x} is —SO₂Me, Ring Q and Ring P form an indazole ring, and X is cyclohexyl as shown, to provide a compound of formula I-f-35:

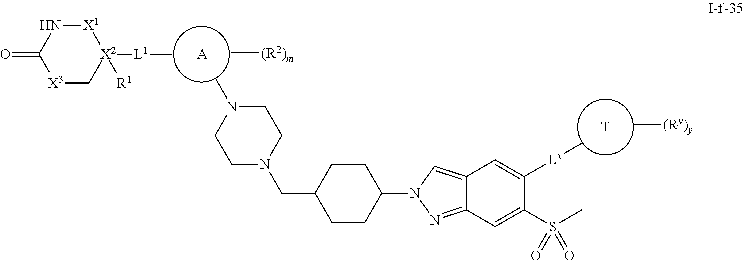

or a pharmaceutically acceptable salt thereof, wherein each of X¹, X², X³, R¹, R², L¹, Ring A, and m of the LBM, and L^{x}, Ring T, R^{y}, and y of the IRAK moiety is as defined above and described in embodiments herein, both singly and in combination.

In some embodiments, the present invention provides the compound of formula I-b′, wherein LBM is

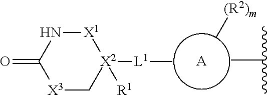

and R¹ is cyclohexyl as shown, to provide a compound of formula I-f-36:

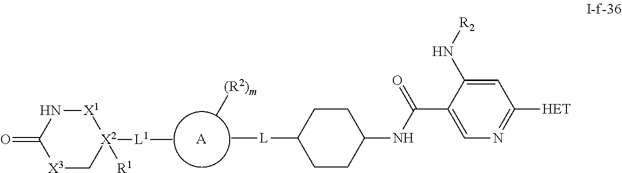

or a pharmaceutically acceptable salt thereof, wherein each of X¹, X², X³, R¹, R², L¹, and Ring A of the LBM, L, and, R₂ and HET of the IRAK moiety is as defined above and described in embodiments herein, both singly and in combination.

In some embodiments, the present invention provides the compound of formula I-b′, wherein LBM is

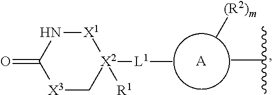

### L is

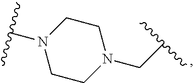 [SMILES: *CN1CCN(*)CC1]

and R₁ is cyclohexyl as shown, to provide a compound of formula I-f-37:

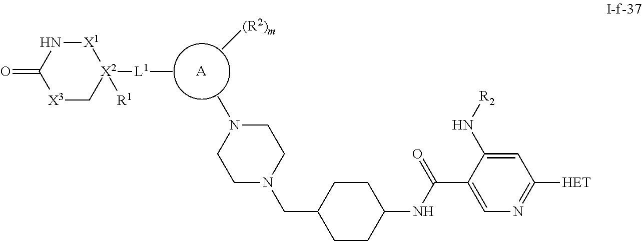

or a pharmaceutically acceptable salt thereof, wherein each of X¹, X², X³, R¹, R², L¹, and Ring A of the LBM, and, R₂ and HET of the IRAK moiety is as defined above and described in embodiments herein, both singly and in combination.

In some embodiments, the present invention provides the compound of formula I-a, wherein LBM is

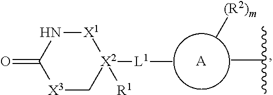

Ring Q and Ring P form an imidazo[1,2-a]pyridine ring, and X is cyclohexyl as shown, to provide a compound of formula I-e-3:

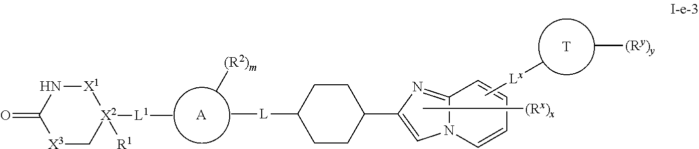

or a pharmaceutically acceptable salt thereof, wherein each of X¹, X², X³, R¹, R². L¹, Ring A, and m of the LBM, L, and L^{x}, Ring T, R, R^{x}, R^{y}, x, and y of the IRAK moiety is as defined above and described in embodiments herein, both singly and in combination.

In some embodiments, the present invention provides the compound of formula I-a, wherein LBM

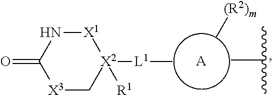

Ring Q is benzo, a single R^{x} is —OR, and X is cyclohexyl as shown, to provide a compound of formula I-e-4:

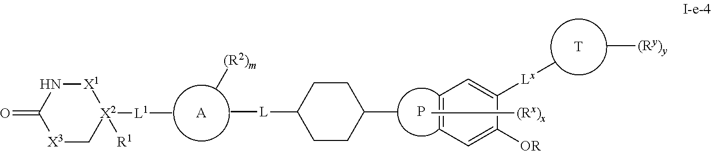

or a pharmaceutically acceptable salt thereof, wherein each of X¹, X², X³, R¹, R², L¹, Ring A, and m of the LBM, L, and L^{x}, Ring P, Ring T, R, R^{x}, R^{y}, x, and y of the IRAK moiety is as defined above and described in embodiments herein, both singly and in combination.

In some embodiments, the present invention provides the compound of formula I-a, wherein LBM is

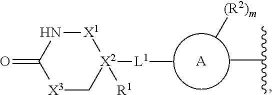

Ring Q and Ring P form an indazole ring, a single R^{x} is —OR, and X is cyclohexyl as shown, to provide a compound of formula I-e-5:

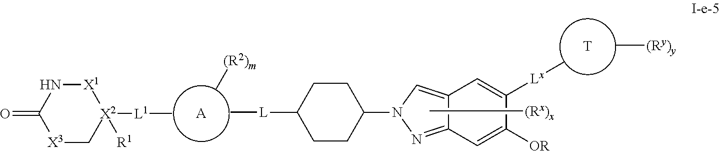

or a pharmaceutically acceptable salt thereof, wherein each of X¹, X², X³, R¹, R², L¹, Ring A, and m of the LBM, L, and L^{x}, Ring T, R, R^{x}, R^{y}, x, and y of the IRAK moiety is as defined above and described in embodiments herein, both singly and in combination.

In some embodiments, the present invention provides the compound of formula I-a, wherein LBM is

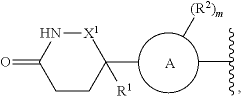

Ring Q is benzo, a single R^{x} is —OR, and X is cyclohexyl as shown, to provide a compound of formula I-e-6:

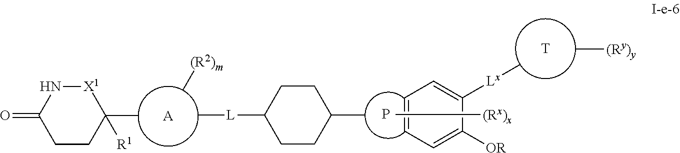

or a pharmaceutically acceptable salt thereof, wherein each of X, R¹, R², Ring A, and m of the LBM, L, and L^{x}, Ring P, Ring T, R, R^{x}, R^{y}, x, and y of the IRAK moiety is as defined above and described in embodiments herein, both singly and in combination.

In some embodiments, the present invention provides the compound of formula I-a, wherein LBM is

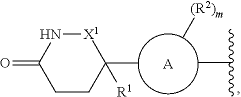

Ring Q and Ring P form an indazole ring, a single R^{x} is —OR, and X is cyclohexyl as shown, to provide a compound of formula I-e-7:

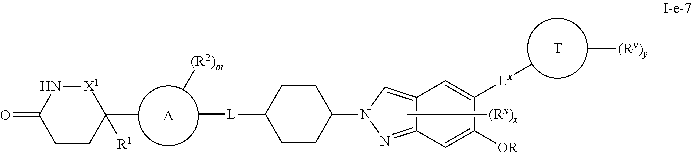

or a pharmaceutically acceptable salt thereof, wherein each of X¹, R¹, R², Ring A, and m of the LBM, L, and L^{x}, Ring T, R, R^{x}, R^{y}, x, and y of the IRAK moiety is as defined above and described in embodiments herein, both singly and in combination.

In some embodiments, the present invention provides the compound of formula I-a, wherein LBM is

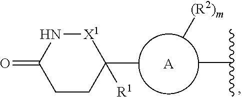

### L is

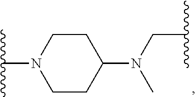 [SMILES: *CN(C)C1CCN(*)CC1]

Ring Q is benzo, a single R^{x} is —OR, and X is cyclohexyl as shown, to provide a compound of formula as a compound of formula I-e-8:

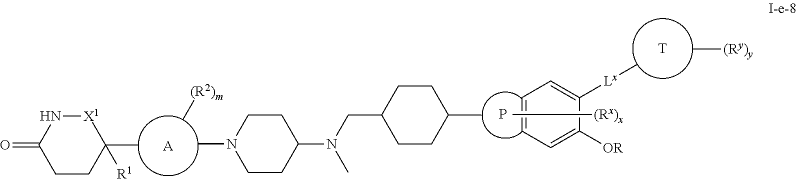

or a pharmaceutically acceptable salt thereof, wherein each of X¹, R¹, R², Ring A, and m of the LBM, and L^{x}, Ring P, Ring T, R, R^{x}, R^{y}, x, and y of the IRAK moiety is as defined above and described in embodiments herein, both singly and in combination.

In some embodiments, the present invention provides the compound of formula I-a, wherein LBM is

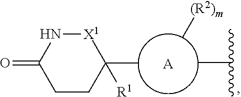

### L is

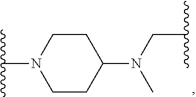 [SMILES: *CN(C)C1CCN(*)CC1]

Ring Q and Ring P form an indazole ring, a single R^{x} is —OR, and X is cyclohexyl as shown, to provide a compound of formula as a compound of formula I-e-9:

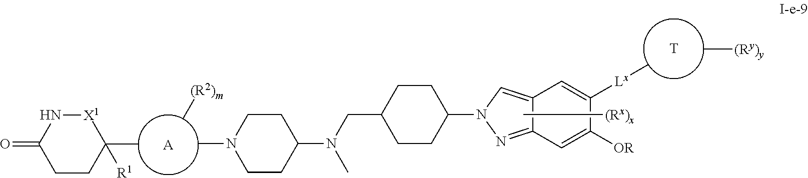

or a pharmaceutically acceptable salt thereof, wherein each of X¹, R¹, R², Ring A, and m of the LBM, and L^{x}, Ring T, R, R^{x}, R^{y}, x, and y of the IRAK moiety is as defined above and described in embodiments herein, both singly and in combination.

In some embodiments, the present invention provides the compound of formula I-a, wherein LBM is

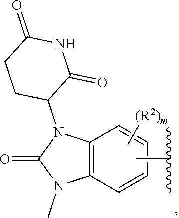

Ring Q is benzo, a single R^{x} is —OR, and X is cyclohexyl as shown, to provide a compound of formula as a compound of formula I-e-10:

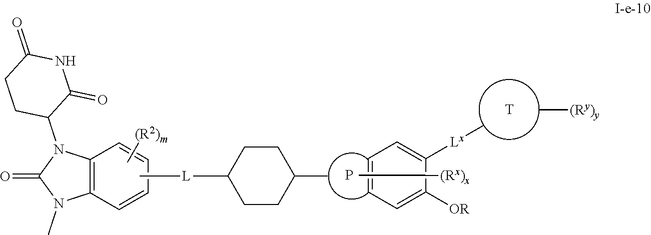

or a pharmaceutically acceptable salt thereof, wherein each of R² and m of the LBM, L, and L^{x}, Ring P, Ring T, R, R^{x}, R^{y}, x, and y of the IRAK moiety is as defined above and described in embodiments herein, both singly and in combination.

In some embodiments, the present invention provides the compound of formula I-a, wherein LBM is

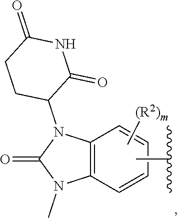

Ring Q and Ring P form an indazole ring, a single R^{x} is —OR, and X is cyclohexyl as shown, to provide a compound of formula as a compound of formula I-e-11:

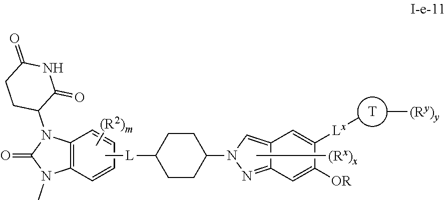

or a pharmaceutically acceptable salt thereof, wherein each of R² and m of the LBM, L, and L^{x}, Ring T, R, R^{x}, R^{y}, x, and y of the IRAK moiety is as defined above and described in embodiments herein, both singly and in combination.

In some embodiments, the present invention provides a compound of formula I-a, wherein LBM is

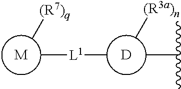

and Ring P and Ring Q form an indazole ring as shown, to provide a compound of formula I-e-12:

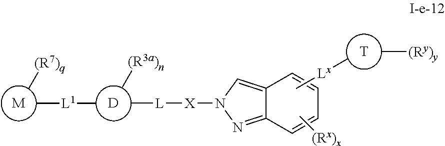

or a pharmaceutically acceptable salt thereof, wherein each of Ring M, Ring D, L, L¹, R^{3a}, R⁷, n, q, X, L^{x}, Ring T, R^{x}, R^{y}, x, and y is as defined above and described in embodiments herein, both singly and in combination.

In some embodiments, the present invention provides a compound of formula I-a, wherein LBM is

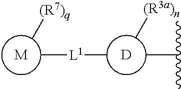

and Ring P and Ring Q form an 6-azaindazole ring as shown, to provide a compound of formula I-e-13:

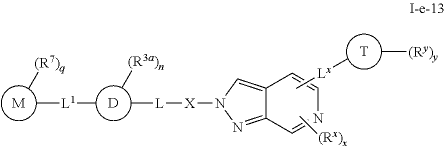

or a pharmaceutically acceptable salt thereof, wherein each of Ring M, Ring D, L, L¹, R^{3a}, R⁷, n, q, X, L^{x}, Ring T, R^{x}, R^{y}, x, and y is as defined above and described in embodiments herein, both singly and in combination.

In some embodiments, the present invention provides a compound of formula I-a, wherein LBM is

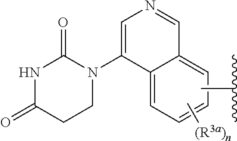

and Ring P and Ring Q form an indazole ring as shown, to provide a compound of formula I-e-14:

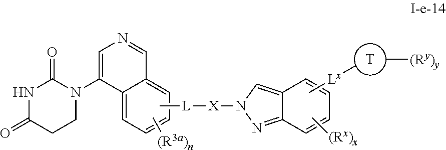

or a pharmaceutically acceptable salt thereof, wherein each of L, R^{3a}, n, X, L^{x}, Ring T, R^{x}, R^{y}, x, and y is as defined above and described in embodiments herein, both singly and in combination.

In some embodiments, the present invention provides a compound of formula I-a, wherein LBM is

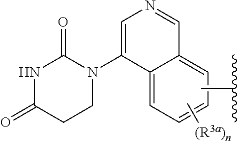

and Ring P and Ring Q form an 6-azaindazole ring as shown, to provide a compound of formula I-e-15:

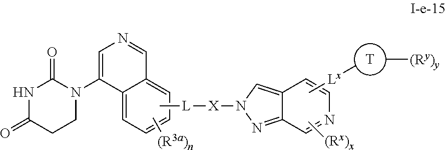

or a pharmaceutically acceptable salt thereof, wherein each of L, R^{3a}, n, X, U, Ring T, R^{x}, R^{y}, x, and y is as defined above and described in embodiments herein, both singly and in combination.

In some embodiments, the present invention provides the compound of formula I-a, wherein LBM is

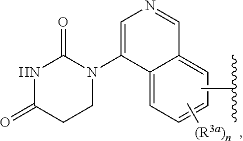

Ring Q is benzo, a single R^{x} is —OR, and X is cyclohexyl as shown, to provide a compound of formula as a compound of formula I-e-16:

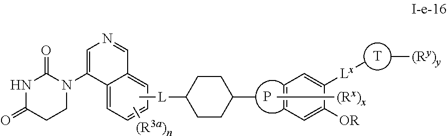

or a pharmaceutically acceptable salt thereof, wherein each of R^{3a} and n of the LBM, L, and L^{x}, Ring P, Ring T, R, R^{x}, R^{y}, x, and y of the IRAK moiety is as defined above and described in embodiments herein, both singly and in combination.

In some embodiments, the present invention provides the compound of formula I-a, wherein LBM is

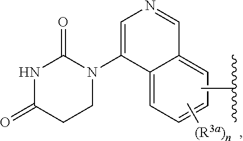

Ring Q and Ring P form an indazole ring, a single R^{x} is —OR, and X is cyclohexyl as shown, to provide a compound of formula as a compound of formula I-e-17:

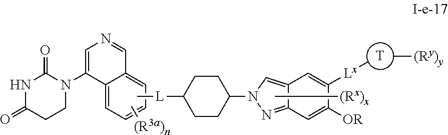

or a pharmaceutically acceptable salt thereof, wherein each of R^{3a} and n of the LBM, L, and L^{x}, Ring T, R, R^{x}, R^{y}, x, and y of the IRAK moiety is as defined above and described in embodiments herein, both singly and in combination.

In some embodiments, the present invention provides the compound of formula I-a, wherein LBM is

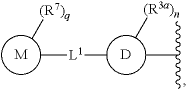

Ring Q and Ring P form an indazole ring, n is 1 and R^{3a} is —OC₁₋₆alkyl, x is 1 and R^{x} is —OMe, and X is cyclohexyl as shown, to provide a compound of formula I-e-18:

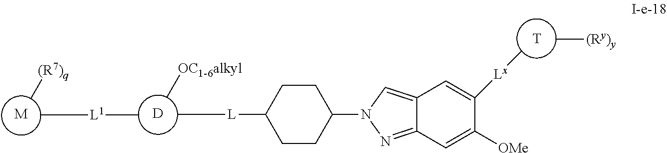

or a pharmaceutically acceptable salt thereof, wherein each of Ring M, Ring D, L, L¹, R⁷, q, L^{x}, Ring T, R^{y}, and y is as defined above and described in embodiments herein, both singly and in combination.

In some embodiments, the present invention provides the compound of formula I-a, wherein LBM is

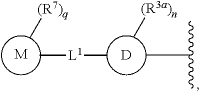

Ring Q and Ring P form an indazole ring, n is 1 and R^{3a} is —OMe, x is 1 and R is —OMe, and X is cyclohexyl as shown, to provide a compound of formula I-e-19:

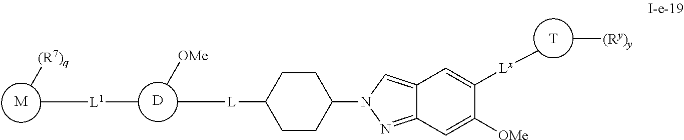

or a pharmaceutically acceptable salt thereof, wherein each of Ring M, Ring D, L, L¹, R⁷, q, L^{x}, Ring T, R^{y}, and y is as defined above and described in embodiments herein, both singly and in combination.

In some embodiments, the present invention provides the compound of formula I-a, wherein LBM is

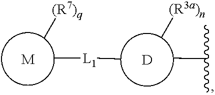

### L is

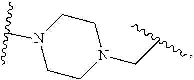 [SMILES: *CN1CCN(*)CC1]

Ring Q and Ring P form an indazole ring, n is 1 and R^{3a} is —OC₁₋₆alkyl, x is 1 and R^{x} is —OMe, and X is cyclohexyl as shown, to provide a compound of formula I-e-20:

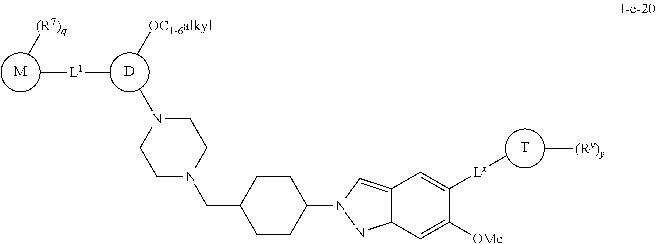

or a pharmaceutically acceptable salt thereof, wherein each of Ring M, Ring D, L, L¹, R⁷, q, L^{x}, Ring T, R^{y}, and y is as defined above and described in embodiments herein, both singly and in combination.

In some embodiments, the present invention provides the compound of formula I-a, wherein LBM is

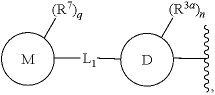

### L is

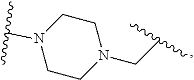 [SMILES: *CN1CCN(*)CC1]

Ring Q and Ring P form an indazole ring, n is 1 and R^{3a} is —OMe, x is 1 and R^{x} is —OMe, and X is cyclohexyl as shown, to provide a compound of formula I-e-21:

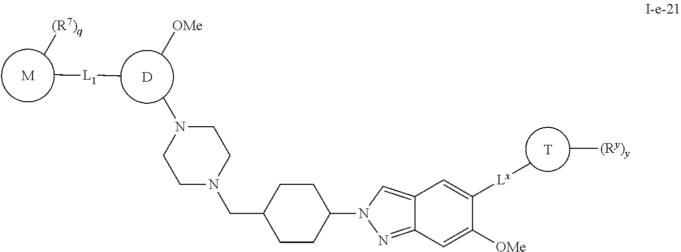

or a pharmaceutically acceptable salt thereof, wherein each of Ring M, Ring D, L, L¹, R⁷, q, L^{x}, Ring T, R^{y}, and y is as defined above and described in embodiments herein, both singly and in combination.

In some embodiments, LBM is an E3 ligase ligand well known to one of ordinary skill in the art including those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

In certain embodiments, the present invention provides a compound of Formula I, wherein LBM is an E3 ubiquitin ligase (cereblon) binding moiety thereby forming a compound of formula I-oo-1, I-oo-2, I-oo-3, I-oo-4, I-oo-5, I-oo-6, I-oo-7, I-oo-8, I-oo-9, or I-oo-10 respectively:

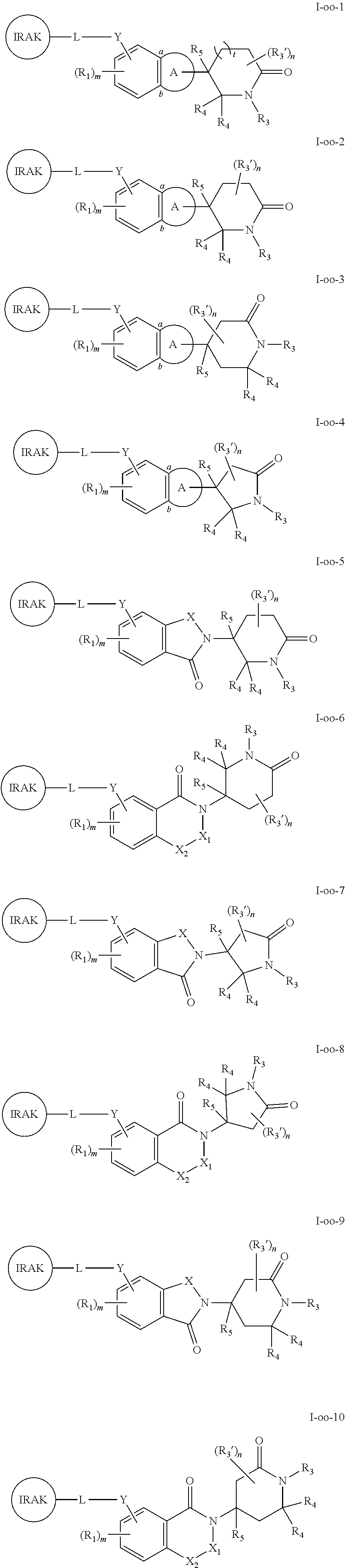

or a compound of formula I-oo′-1, I-oo′-2, I-oo′-3, I-oo′-4, I-oo′-5, I-oo′-6, I-oo′-7, I-oo′-8, I-oo′-9, or I-oo′-10 respectively:

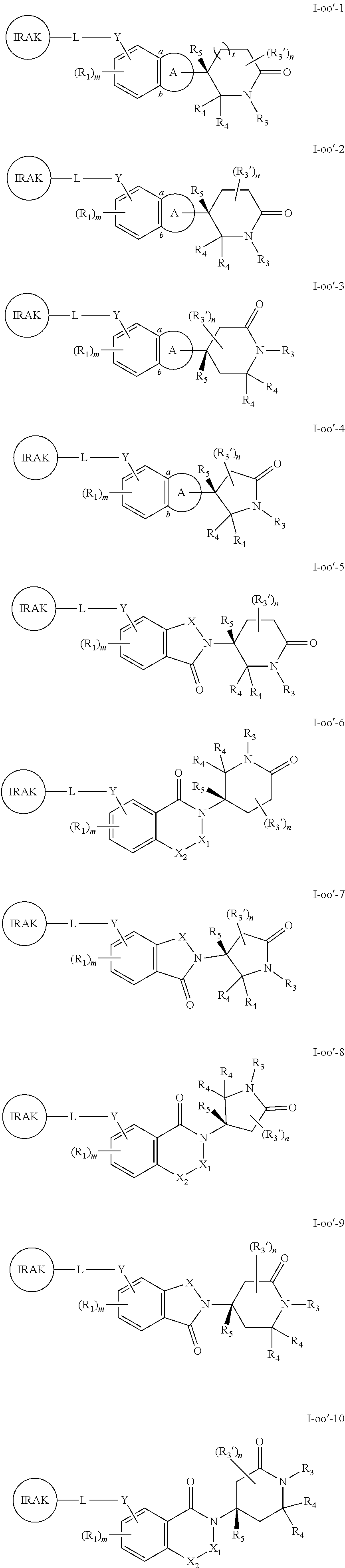

or a compound of formula I-oo″-1, I-oo″-2, I-oo″-3, I-oo″-4, I-oo″-5, I-oo″-6, I-oo″-7, I-oo″-8, I-oo″-9, or I-oo″-10 respectively:

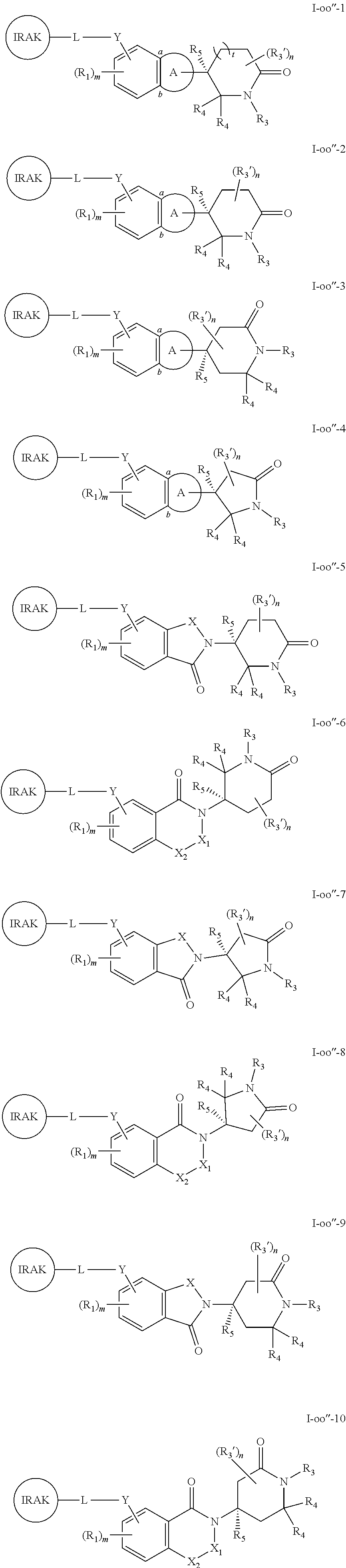

or a pharmaceutically acceptable salt thereof, wherein L and IRAK are as defined above and described in embodiments herein, and wherein each of the variables

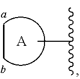

X, X₁, X₂, Y, R₁, R₃, R₃′, R₄, R₅, t, m and n is as defined and described in WO 2017/007612 and US 2018/0134684, the entirety of each of which is herein incorporated by reference.

Accordingly in some embodiments, the present invention provides a compound of formula I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, or I-oo″-10, or a pharmaceutically acceptable salt thereof, wherein:

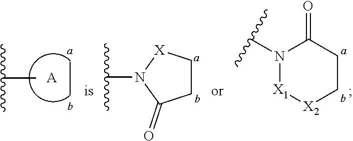

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁—X₂ is C(R₃)═N or C(R₃)₂—C(R₃)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆alkyl, C₁-C₆haloalkyl, C₁-C₆alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

In certain embodiments, the present invention provides a compound of Formula I, wherein LBM is an E3 ubiquitin ligase (cereblon) binding moiety thereby forming a compound of formula I-pp-1, I-pp-2, I-pp-3, I-pp-4, I-pp-5, or I-pp-6 respectively:

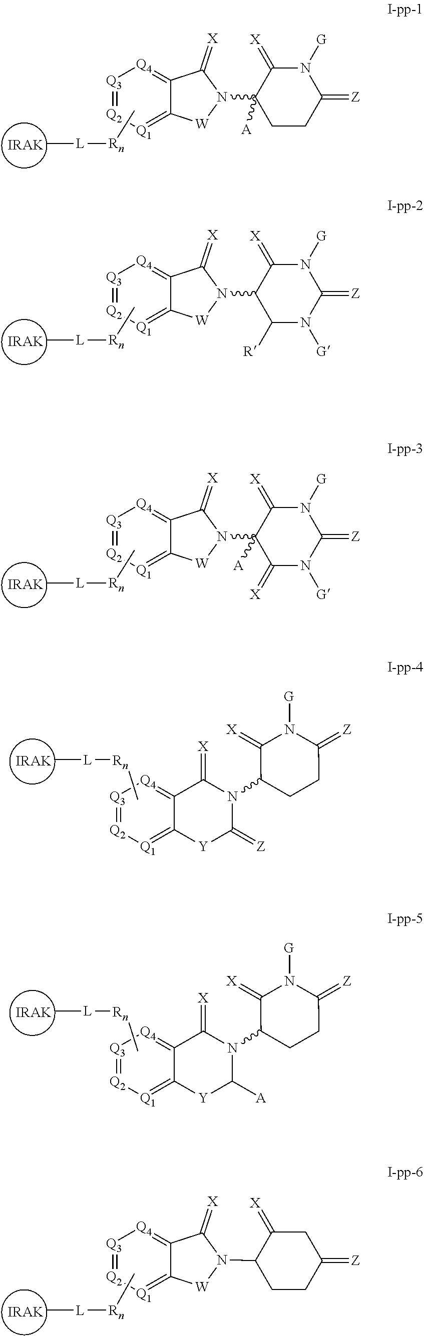

or a pharmaceutically acceptable salt thereof, wherein L and IRAK are as defined above and described in embodiments herein, and wherein each of the variables A, G, G′, Q₁, Q₂, Q₃, Q₄, R, R′, W, X, Y, Z, [symbol] , and n is as defined and described in WO 2016/197114 and US 2018/0147202, the entirety of each of which is herein incorporated by reference.

In some embodiments, LBM is

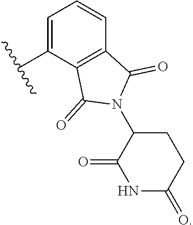 [SMILES: *c1cccc2c1C(=O)N(C1CCC(=O)NC1=O)C2=O]

In some embodiments, LBM is

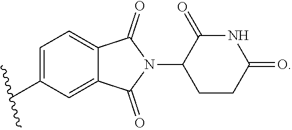 [SMILES: *c1ccc2c(c1)C(=O)N(C1CCC(=O)NC1=O)C2=O]

In some embodiments, LBM is

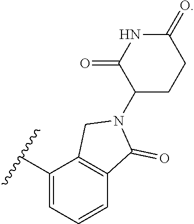 [SMILES: *c1cccc2c1CN(C1CCC(=O)NC1=O)C2=O]

In some embodiments, LBM is

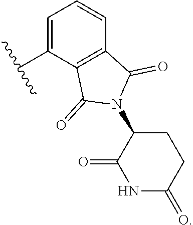 [SMILES: *c1cccc2c1C(=O)N([C@H]1CCC(=O)NC1=O)C2=O]

In some embodiments, LBM is

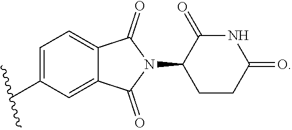 [SMILES: *c1ccc2c(c1)C(=O)N([C@@H]1CCC(=O)NC1=O)C2=O]

In some embodiments, LBM is

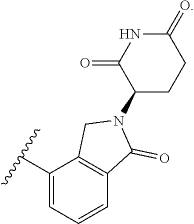 [SMILES: *c1cccc2c1CN([C@@H]1CCC(=O)NC1=O)C2=O]

In some embodiments, LBM is

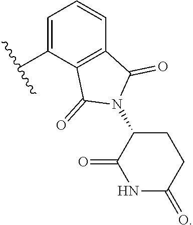 [SMILES: *c1cccc2c1C(=O)N([C@@H]1CCC(=O)NC1=O)C2=O]

In some embodiments, LBM is

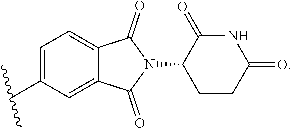 [SMILES: *c1ccc2c(c1)C(=O)N([C@H]1CCC(=O)NC1=O)C2=O]

In some embodiments, LBM is

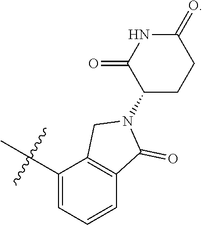 [SMILES: *c1cccc2c1CN([C@H]1CCC(=O)NC1=O)C2=O]

In some embodiments, LBM is selected from those in Table 1 below.

In some embodiments, the present invention provides a compound of formula I-a, wherein LBM is thalidomide and Ring P and Ring Q form a benzoxazole ring as shown, to provide a compound of formula I-j-1:

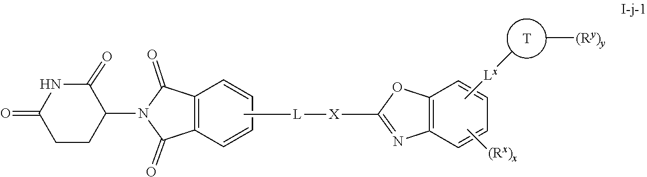

or a pharmaceutically acceptable salt thereof, wherein each of L, L^{x}, X, R^{x}, R^{y}, Ring T, x, and y is as defined above and described in embodiments herein, both singly and in combination.

In some embodiments, the present invention provides a compound of formula I-a, wherein LBM is thalidomide and Ring P and Ring Q form an indazole ring as shown, to provide a compound of formula I-j-3:

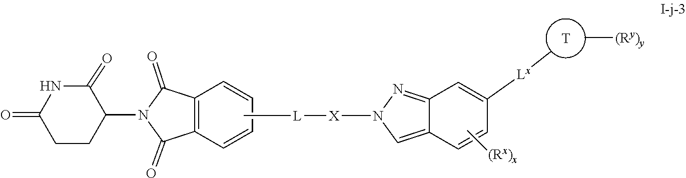

or a pharmaceutically acceptable salt thereof, wherein each of L, L^{x}, X, R^{x}, R^{y}, Ring T, x, and y is as defined above and described in embodiments herein, both singly and in combination.

In some embodiments, the present invention provides a compound of formula I-a, wherein LBM is thalidomide and Ring P and Ring Q form a imidazo[1,2-a]pyridine ring as shown, to provide a compound of formula I-j-4:

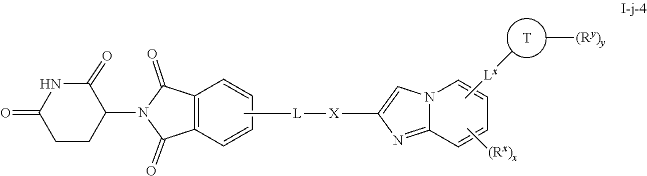

or a pharmaceutically acceptable salt thereof, wherein each of L, L^{x}, X, R^{x}, R^{y}, Ring T, x, and y is as defined above and described in embodiments herein, both singly and in combination.

In some embodiments, the present invention provides a compound of formula I-a, wherein LBM is thalidomide or

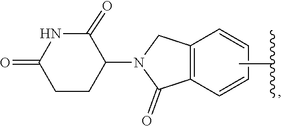

### Ring Q is

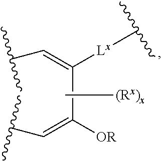

a single R^{x} is —OR, and X is cyclohexyl as shown, to provide a compound of formula I-k-2 or I-k-3:

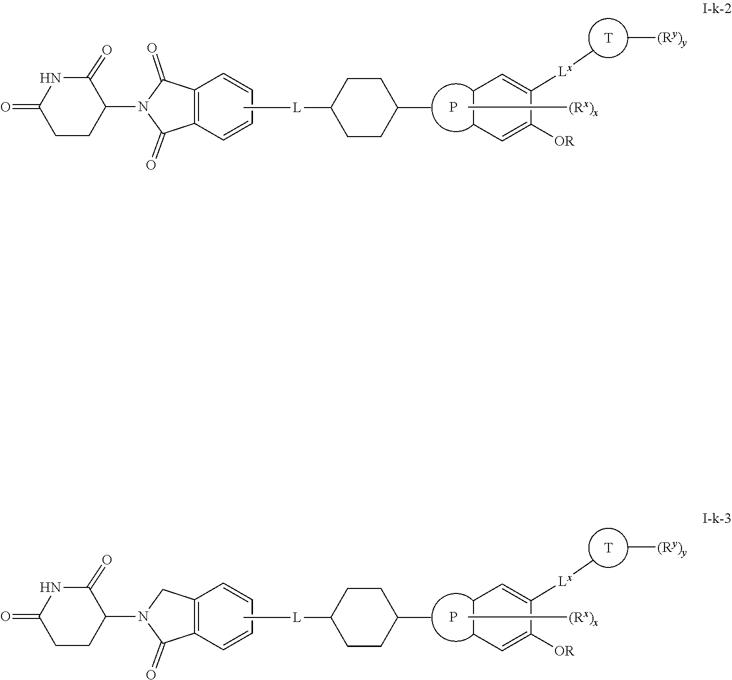

or a pharmaceutically acceptable salt thereof, wherein L is as defined and described herein, and wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R², or

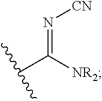

or
   two R^{x} groups are optionally taken together to form an optionally substituted 5-6 membered partially unsaturated or aryl fused ring having 0-3 heteroatoms independently selected from nitrogen, oxygen, or sulfur;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-7 membered saturated, partially unsaturated, or heteroaryl ring having 0-3 heteroatoms, in addition to the carbon or nitrogen, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or NR₂

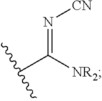

each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring P is selected from benzo, a 4-7 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring P is optionally substituted with 1-2 oxo groups;
Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups;
L^{x} is a covalent bond or a C₁₋₃ bivalent straight or branched saturated or unsaturated hydrocarbon chain wherein 1-2 methylene units of the chain are independently and optionally replaced with -Cy^{x}-, —O—, —S—, —C(O)—, —C(S)—, —CR₂—, —CRF—, —CF₂—, —NR—, —N═CR—, —CR═CR—, or —S(O)₂—, wherein R of —CR₂—, —CRF—, —NR—, —N═CR—, or —CR═CR— can combine with R^{x} or R^{y} to form a 4-7 membered saturated or partially unsaturated carbocyclic or heterocyclic ring having 1-3 heteroatoms independently selected from nitrogen, oxygen, and sulfur;
Cy^{x}- is an optionally substituted ring selected from a 3-5 membered saturated or partially unsaturated carbocyclic or heterocyclic ring having 0-3 heteroatoms independently selected from nitrogen, oxygen, and sulfur, or a 5 membered heteroaryl ring having 1-4 heteroatoms independently selected from nitrogen, oxygen, and sulfur, wherein -Cy^{x}- is optionally substituted with 1-2 oxo groups;
each x is 0, 1, 2, 3 or 4; and
each y is 0, 1, 2, 3 or 4.

In certain embodiments, the present invention provides a compound of formula I-k-2 or I-k-3 above, wherein L^{x} is amide, Ring P is pyrazolyl, Ring T is pyridyl, R is Me, and R^{y} is —CF₃.

In some embodiments, the present invention provides a compound of formula I-a, wherein LBM is thalidomide or

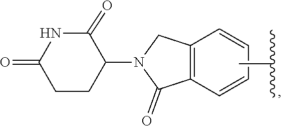

Ring P and Ring Q form an indazole ring, a single R^{x} is —OR, and X is cyclohexyl as shown, to provide a compound of formula I-k-4 or I-k-5:

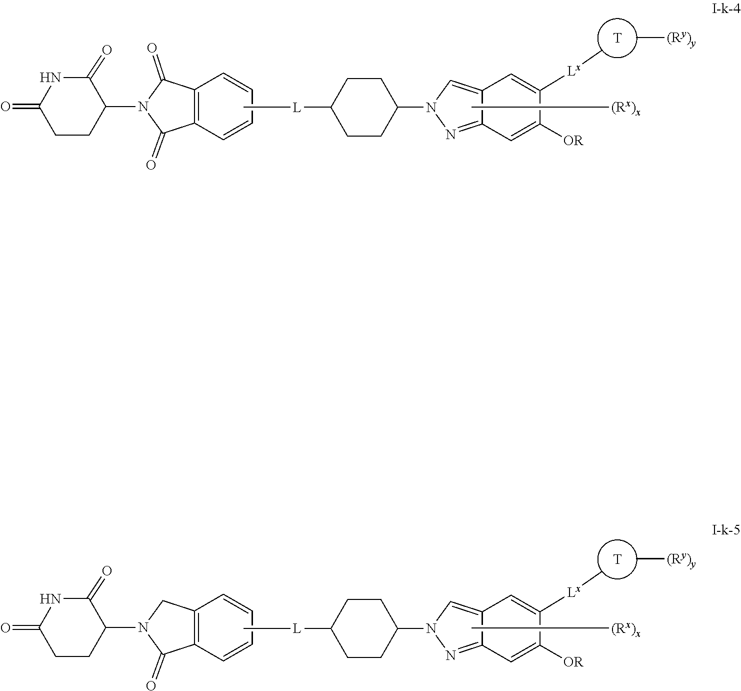

or a pharmaceutically acceptable salt thereof, wherein L is as defined and described herein, and wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R², or NR₂

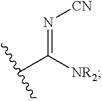

or
   two R^{x} groups are optionally taken together to form an optionally substituted 5-6 membered partially unsaturated or aryl fused ring having 0-3 heteroatoms independently selected from nitrogen, oxygen, or sulfur;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-7 membered saturated, partially unsaturated, or heteroaryl ring having 0-3 heteroatoms, in addition to the carbon or nitrogen, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or

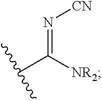

each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups;
L^{x} is a covalent bond or a C₁₋₃ bivalent straight or branched saturated or unsaturated hydrocarbon chain wherein 1-2 methylene units of the chain are independently and optionally replaced with -Cy^{x}-, —O—. —S—, —C(O)—, —C(S)—, —CR₂—, —CRF—, —CF₂—, —NR—, —N═CR—, —CR═CR—, or —S(O)₂—, wherein R of —CR₂—, —CRF—, —NR—, —N═CR—, or —CR═CR— can combine with R^{x} or R^{y} to form a 4-7 membered saturated or partially unsaturated carbocyclic or heterocyclic ring having 1-3 heteroatoms independently selected from nitrogen, oxygen, and sulfur;
Cy^{x}- is an optionally substituted ring selected from a 3-5 membered saturated or partially unsaturated carbocyclic or heterocyclic ring having 0-3 heteroatoms independently selected from nitrogen, oxygen, and sulfur, or a 5 membered heteroaryl ring having 1-4 heteroatoms independently selected from nitrogen, oxygen, and sulfur, wherein -Cy^{x}- is optionally substituted with 1-2 oxo groups;
each x is 0, 1, 2, 3 or 4; and
each y is 0, 1, 2, 3 or 4.

In certain embodiments, the present invention provides a compound of formula I-k-6 or I-k-7 above, wherein L^{x} is amide, Ring T is pyridyl, R is Me, and R^{y} is —CF₃.

In some embodiments, the present invention provides a compound of formula I-a, wherein LBM is thalidomide or

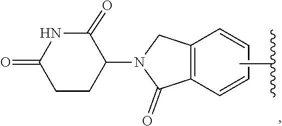

### Ring Q is OR

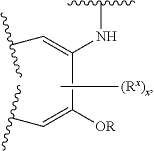

a, L^{x} is amide, a single R^{x} is —OR, and X is cyclohexyl as shown, to provide a compound of formula I-k-6 or I-k-7:

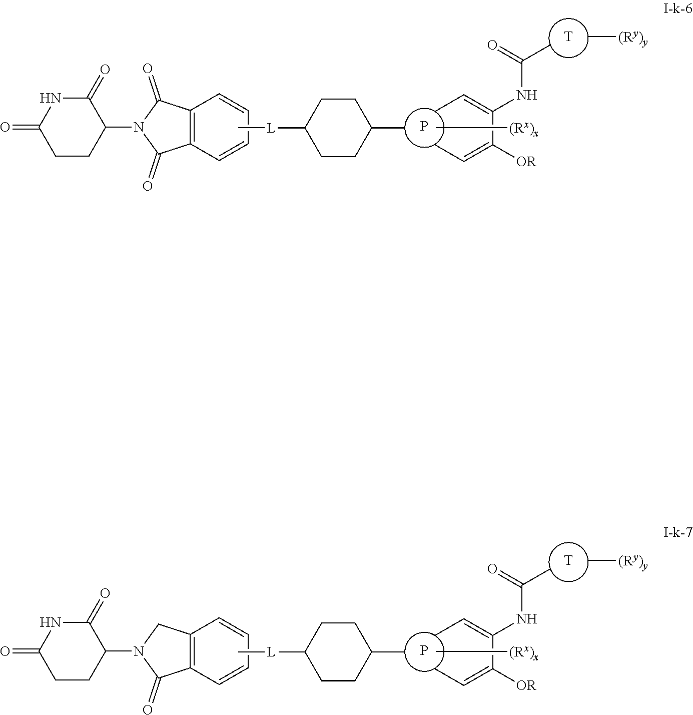

or a pharmaceutically acceptable salt thereof, wherein L is as defined and described herein, and wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂F, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R², or NR₂

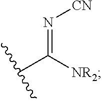

or
   two R groups are optionally taken together to form an optionally substituted 5-6 membered partially unsaturated or aryl fused ring having 0-3 heteroatoms independently selected from nitrogen, oxygen, or sulfur;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-7 membered saturated, partially unsaturated, or heteroaryl ring having 0-3 heteroatoms, in addition to the carbon or nitrogen, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or

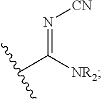

each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring P is selected from benzo, a 4-7 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring P is optionally substituted with 1-2 oxo groups;
Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups;
each x is 0, 1, 2, 3 or 4; and
each y is 0, 1, 2, 3 or 4.

In certain embodiments, the present invention provides a compound of formula I-k-6 or I-k-7 above, wherein Ring P is pyrazolyl, Ring T is pyridyl, R is Me, and R^{y} is —CF₃.

In some embodiments, the present invention provides a compound of formula I-a, wherein LBM is thalidomide or

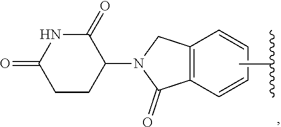

Ring P and Ring Q form an indazole ring, L^{x} is amide, a single R is —OR, and X is cyclohexyl as shown, to provide a compound of formula I-k-8 or I-k-9:

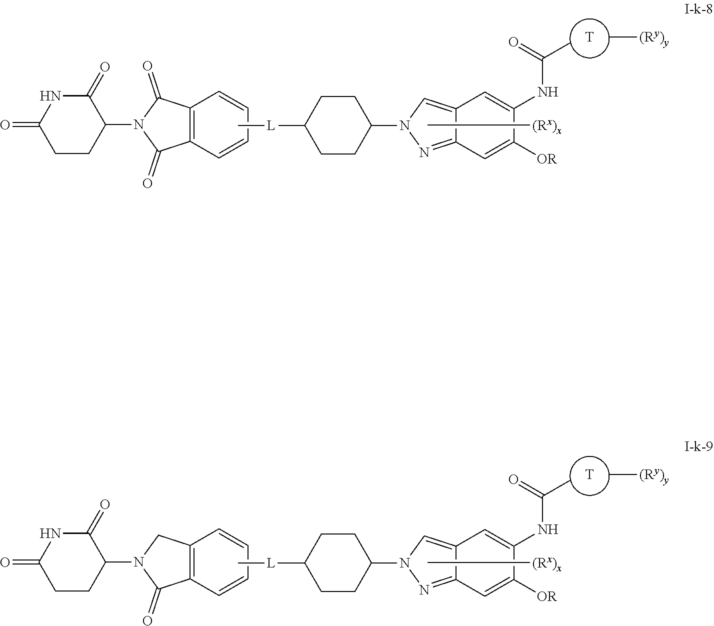

or a pharmaceutically acceptable salt thereof, wherein L is as defined and described herein, and wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R², or

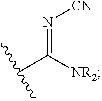

or
   two R^{x} groups are optionally taken together to form an optionally substituted 5-6 membered partially unsaturated or aryl fused ring having 0-3 heteroatoms independently selected from nitrogen, oxygen, or sulfur;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-7 membered saturated, partially unsaturated, or heteroaryl ring having 0-3 heteroatoms, in addition to the carbon or nitrogen, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or

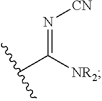

each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups;
each x is 0, 1, 2, 3 or 4; and
each y is 0, 1, 2, 3 or 4.

In certain embodiments, the present invention provides a compound of formula I-k-8 or I-k-9 above, wherein Ring T is pyridyl, R is Me, and R^{y} is —CF₃.

In some embodiments, the present invention provides a compound of formula I-a, wherein LBM is thalidomide or

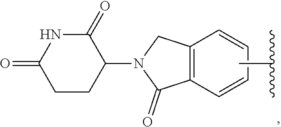

Ring P and Ring Q form an indazole ring, Ring T is pyridyl, L^{x} is amide, a single R^{x} is —OR, and X is cyclohexyl as shown, to provide a compound of formula I-k-10 or I-k-11:

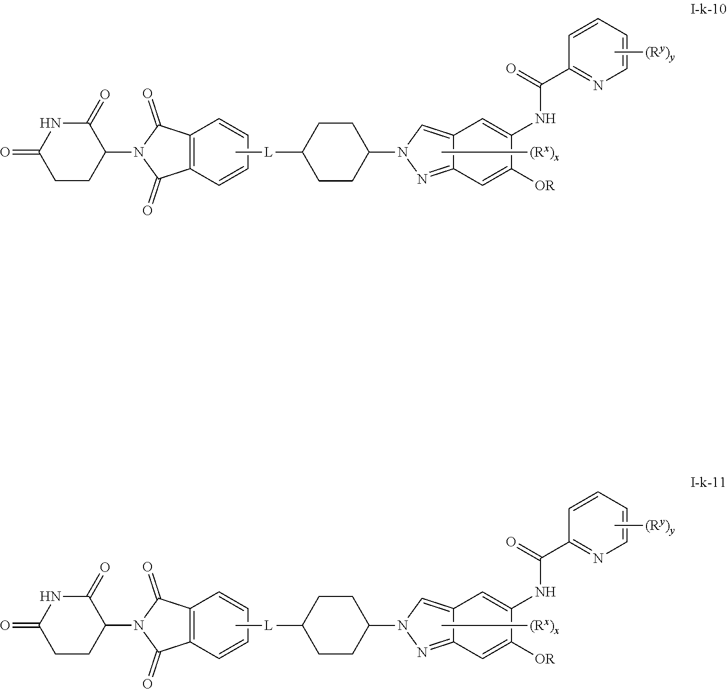

or a pharmaceutically acceptable salt thereof, wherein L is as defined and described herein, and wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R², or

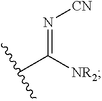

or
   two R^{x} groups are optionally taken together to form an optionally substituted 5-6 membered partially unsaturated or aryl fused ring having 0-3 heteroatoms independently selected from nitrogen, oxygen, or sulfur;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-7 membered saturated, partially unsaturated, or heteroaryl ring having 0-3 heteroatoms, in addition to the carbon or nitrogen, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or

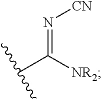

each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each x is 0, 1, 2, 3 or 4; and
each y is 0, 1, 2, 3 or 4.

In certain embodiments, the present invention provides a compound of formula I-k-10 or I-k-11 above, wherein R is Me and R^{y} is —CF₃.

In some embodiments, the present invention provides a compound of formula I-a, wherein LBM is

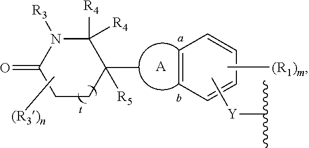

### Ring Q is

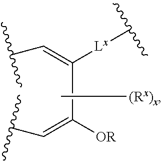

a single R is —OR, and X is cyclohexyl as shown, to provide a compound of formula I-k-12:

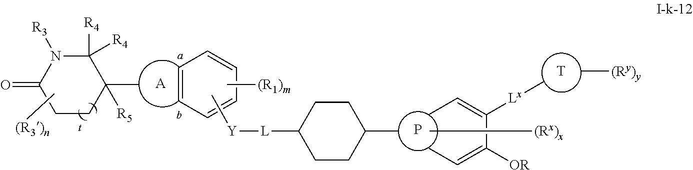

or a pharmaceutically acceptable salt thereof, wherein each of variables

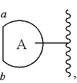

Y, R₁, R₃, R₃′, R₄, R₅, t, m, n, Ring P, Ring T, L, L^{x}, R^{x}, R^{y}, x, and y is as defined above and described in embodiments herein, both singly and in combination.

In some embodiments, the present invention provides a compound of formula I-a, wherein LBM is

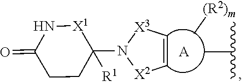

### Ring Q is

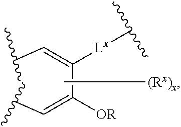

a single R^{x} is —OR, and X is cyclohexyl as shown, to provide a compound of formula I-k-13:

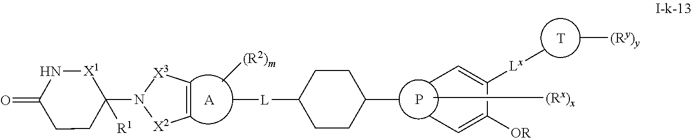

or a pharmaceutically acceptable salt thereof, wherein each of variables X¹, X², X³, R¹, R², Ring A, m, Ring P, Ring T, L, L, R^{x}, R^{y}, x, and y is as defined above and described in embodiments herein, both singly and in combination.

In some embodiments, the present invention provides a compound of formula I-a, wherein LBM is

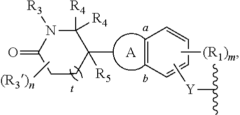

Ring P and Ring Q form an indazole ring, a single R is —OR, and X is cyclohexyl as shown, to provide a compound of formula I-k-14:

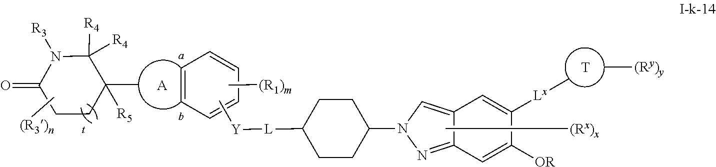

or a pharmaceutically acceptable salt thereof, wherein each of variables

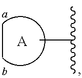

Y, R₁, R₃, R₃′, R⁴, R₅, t, m, n, Ring T, L, L^{x}, R^{x}, R^{y}, x, and y is as defined above and described in embodiments herein, both singly and in combination.

In some embodiments, the present invention provides a compound of formula I-a, wherein LBM is

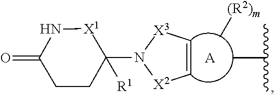

Ring P and Ring Q form an indazole ring, a single R^{x} is —OR, and X is cyclohexyl as shown, to provide a compound of formula I-k-15:

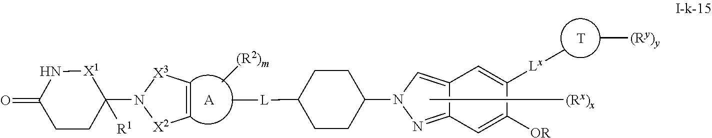

or a pharmaceutically acceptable salt thereof, wherein each of variables X¹, X², X³, R¹, R², Ring A, m, Ring T, L, L^{x}, R^{x}, R^{y}, x, and y is as defined above and described in embodiments herein, both singly and in combination.

In certain embodiments, the present invention provides a compound of Formula I, wherein LBM is an E3 ubiquitin ligase (cereblon) binding moiety thereby forming a compound of formula I-qq-1, I-qq-2, or I-qq-3 respectively:

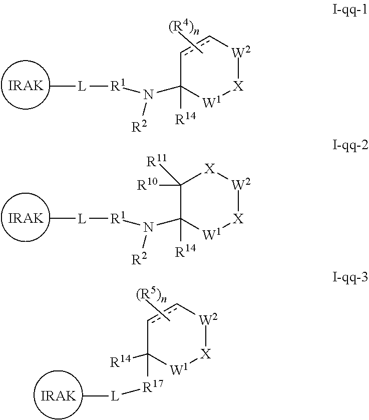

or a pharmaceutically acceptable salt thereof, wherein L and IRAK are as defined above and described herein, and wherein each of the variables R¹, R², R⁴, R⁵, R¹⁰, R¹¹, R¹⁴, R¹⁷, W¹, W², X, [symbol] , and n is as defined in WO 2017/197051 which is herein incorporated by reference in its entirety and wherein

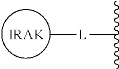

is attached to R¹, the ring formed by combining R¹ and R², or R¹⁷ at the site of attachment of R¹² as defined in WO 2017/197051 such that

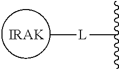

takes the place of the R¹² substituent.

In some embodiments, the present invention provides a compound of formula I, wherein LBM is an E3 ubiquitin ligase (cereblon) binding moiety thereby forming a compound of formula I-rr-1, I-rr-2, I-rr-3, or I-rr-4, respectively:

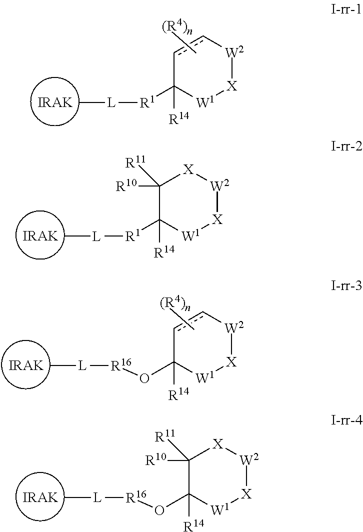

or a pharmaceutically acceptable salt thereof, wherein L and IRAK are as defined above and described herein, and wherein each of the variables R¹, R⁴, R¹⁰, R¹¹, R¹⁴, R¹⁶, W¹, W², X, [symbol] , an n is as defined in WO 2018/237026, the entirety of each of which is herein incorporated by reference, and wherein

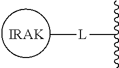

is attached to R¹ or R¹⁶ at the site of attachment of R¹² as defined in WO 2018/237026, such that

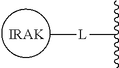

takes the place of the R¹² substituent.

In some embodiments, the present invention provides a compound of formula I, wherein LBM is an E3 ubiquitin ligase (cereblon) binding moiety thereby forming a compound of formula I-ss-1 or I-ss-3, respectively:

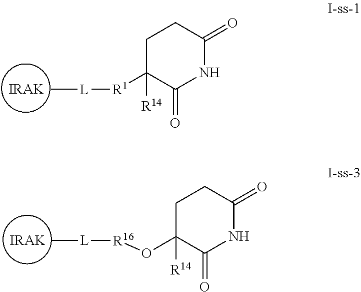

or a pharmaceutically acceptable salt thereof, wherein L and IRAK are as defined above and described herein, and wherein each of the variables R¹, R¹⁴, and R¹⁶ is as defined in WO 2018/237026, the entirety of each of which is herein incorporated by reference, and wherein

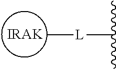

is attached to R¹ or R¹⁶ at the site of attachment of R¹² as defined in WO 2018/237026, such that

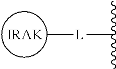

takes the place of the R¹² substituent.

In certain embodiments, the present invention provides a compound of formula I, wherein LBM is an E3 ubiquitin ligase (cereblon) binding moiety thereby forming a compound of formula I-tt-1, I-tt-2, I-tt-3, I-tt-4, I-tt-5, I-tt-6, I-tt-7, or I-tt-8:

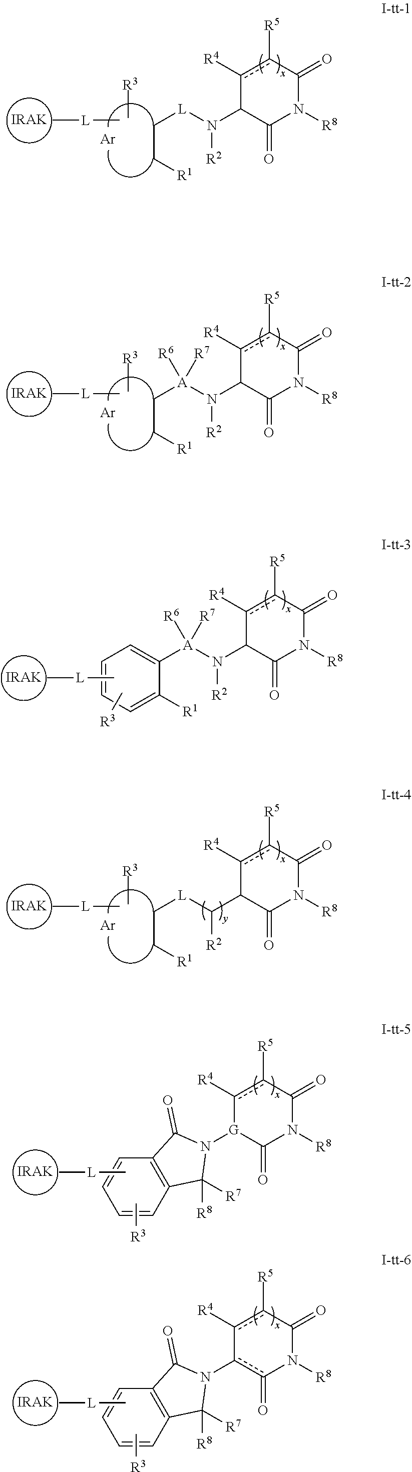

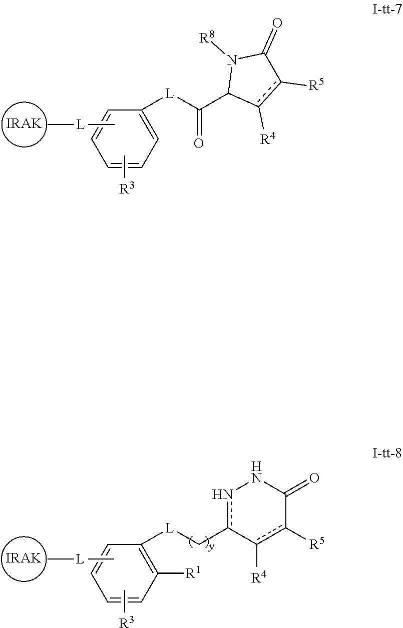

or a pharmaceutically acceptable salt thereof, wherein L and IRAK are as defined above and described in embodiments herein, and wherein each of the variables Ar, R¹, R², R³, R⁴, R⁵, R⁶, R⁷, R⁸, A, L, x, y and [symbol] is as described and defined in WO 2017/161119, the entirety of each of which is herein incorporated by reference.

In certain embodiments, the present invention provides a compound of formula I, wherein LBM is an E3 ubiquitin ligase (cereblon) binding moiety thereby forming a compound of formula I-uu:

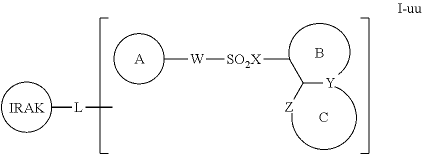

or a pharmaceutically acceptable salt thereof, wherein L and IRAK are as defined above and described in embodiments herein, and wherein each of the variables A, B, C, W, X, Y, and Z is as described and defined in U.S. Pat. No. 5,721,246, the entirety of each of which is herein incorporated by reference.

In certain embodiments, the present invention provides a compound of formula I, wherein LBM is an E3 ubiquitin ligase (cereblon) binding moiety thereby forming a compound of formula I-vv:

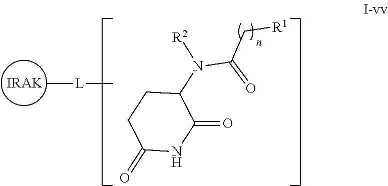

or a pharmaceutically acceptable salt thereof, wherein L and IRAK are as defined above and described in embodiments herein, and wherein each of the variables R¹, R², and n is as described and defined in WO 2019/043214, the entirety of each of which is herein incorporated by reference.

In some embodiments, LBM is a IAP E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

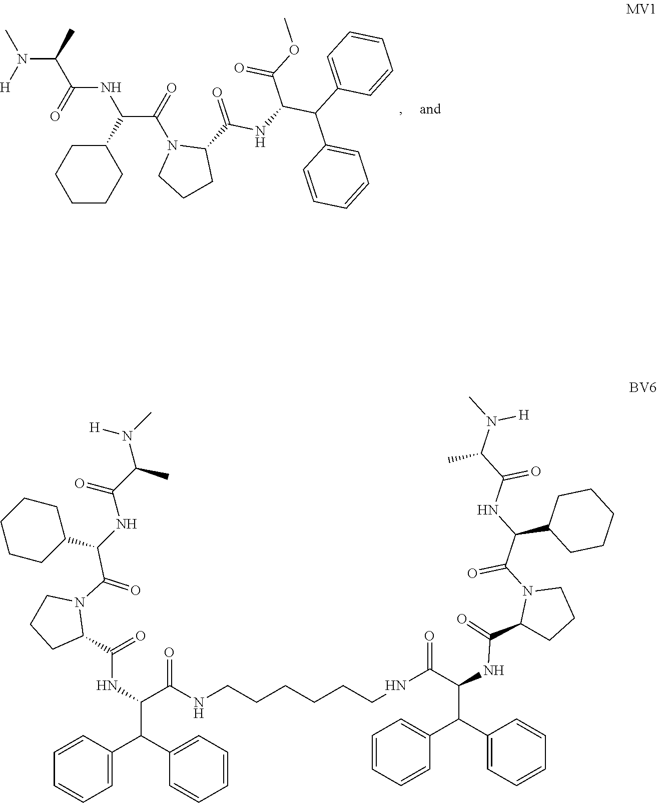

wherein

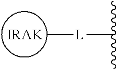

is attached to a modifiable carbon, oxygen, nitrogen or sulfur atom.

In certain embodiments, the present invention provides a compound of Formula I, wherein LBM is a VHL E3 ubiquitin ligase binding moiety thereby forming a compound of formula I-ww-1, I-ww-2, I-ww-3, I-ww-4, or I-ww-5 respectively:

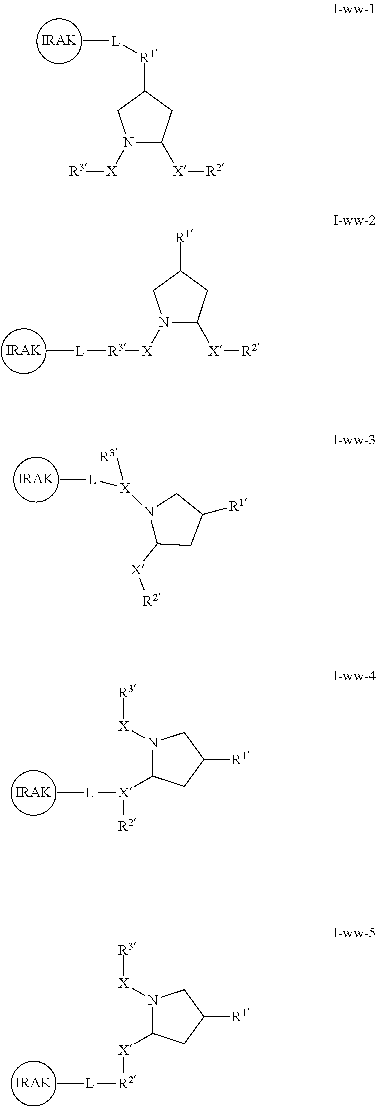

or a pharmaceutically acceptable salt thereof, wherein L and IRAK are as defined above and described in embodiments herein, and wherein each of the variables R^{1′}, R^{2′}, R^{3′}, X, and X′ is as defined and described in WO 2013/106643 and US 2014/0356322, the entirety of each of which is herein incorporated by reference.

In certain embodiments, the present invention provides a compound of Formula I, wherein LBM is a VHL E3 ubiquitin ligase binding moiety thereby forming a compound of formula I-xx-1, I-xx-2, I-xx-3, I-xx-4, I-xx-5 or I-xx-6 respectively:

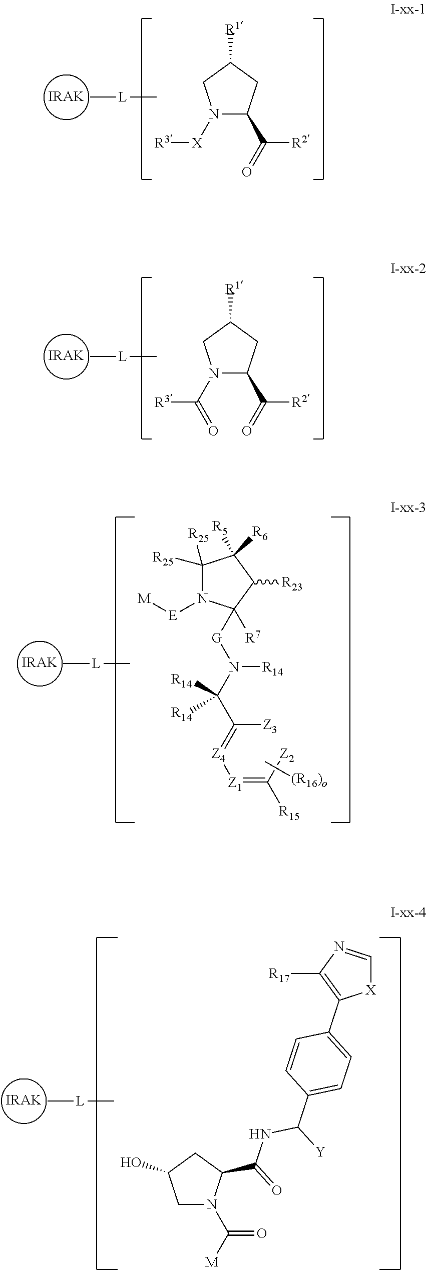

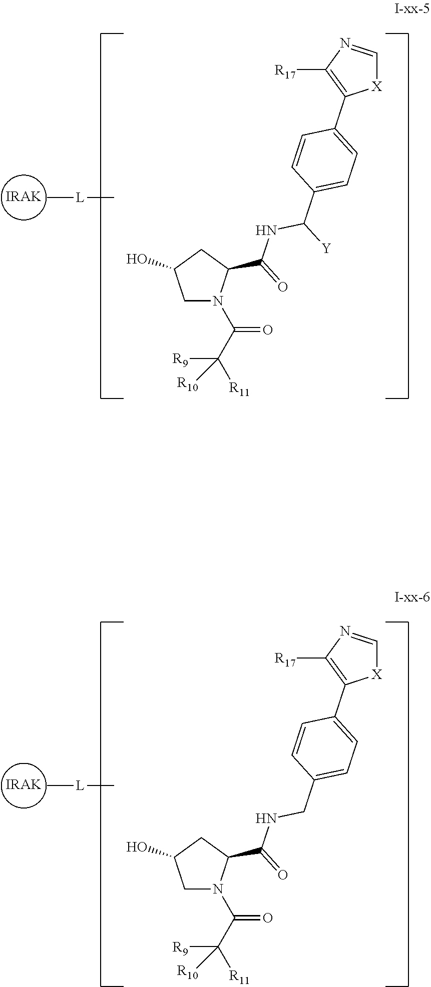

or a pharmaceutically acceptable salt thereof, wherein L and IRAK are as defined above and described in embodiments herein, and wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is as defined and described in WO 2016/149668 and US 2016/0272639, the entirety of each of which is herein incorporated by reference.

As used herein, depiction of brackets around any LBM

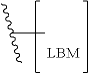

means that the

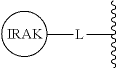

moiety is covalently attached to said LBM at any available modifiable carbon, nitrogen, oxygen, or sulfur atom. For purposes of clarity and by way of example, such available modifiable carbon, nitrogen, oxygen, or sulfur atoms in the following LBM compound structure are depicted below,

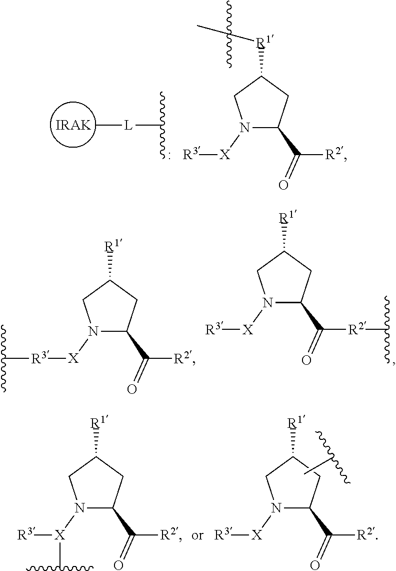

In certain embodiments, the present invention provides a compound of Formula I, wherein LBM is a VHL E3 ubiquitin ligase binding moiety thereby forming a compound of formula I-yy-1, I-yy-2, or I-yy-3 respectively:

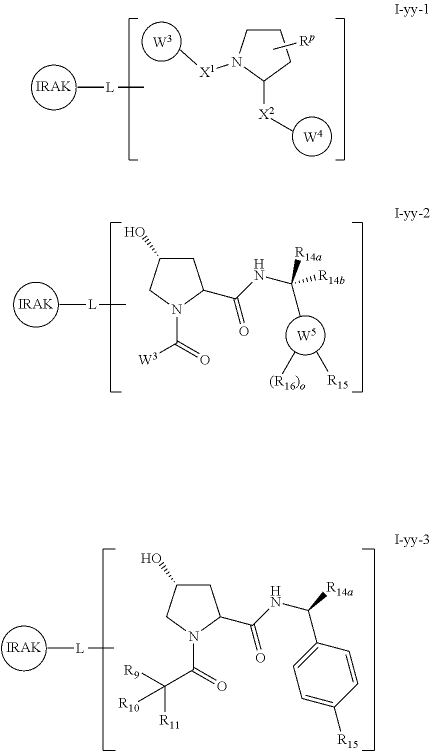

or a pharmaceutically acceptable salt thereof, wherein L and IRAK are as defined above and described in embodiments herein, and wherein each of the variables R^{P}, R₉, R₁₀, R₁₁, R₁₄ₐ, R_{14b}, R₁₅, R₁₆, W³, W⁴, W⁵, X¹, X², and o is as defined and described in WO 2016/118666 and US 2016/0214972, the entirety of each of which is herein incorporated by reference.

In certain embodiments, the present invention provides a compound of Formula I, wherein LBM is a CRBN or VHL E3 ubiquitin ligase binding moiety thereby forming a compound of formula I-zz-1, I-zz-2, I-zz-3, I-zz-4, I-zz-5, I-zz-6, or I-zz-7 respectively:

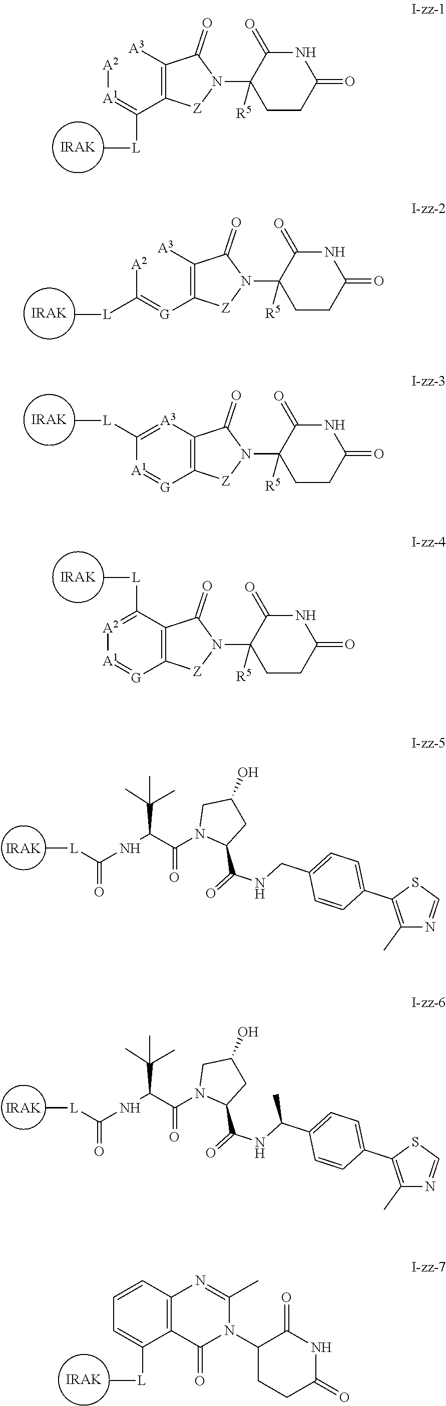

or a pharmaceutically acceptable salt thereof, wherein L and IRAK are as defined above and described in embodiments herein, and wherein each of the variables A¹, A², A³, R⁵, G and Z is as defined and described in WO 2017/176958.

In certain embodiments, the present invention provides a compound of Formula I, wherein LBM is a CRBN E3 ubiquitin ligase binding moiety thereby forming a compound of formula I-zz′-1, I-zz″-1, I-zz′-2, I-zz′-2, I-zz′-3, I-zz″-3, I-zz′-4, I-zz″-4, I-zz′-7 or I-zz″-7 respectively:

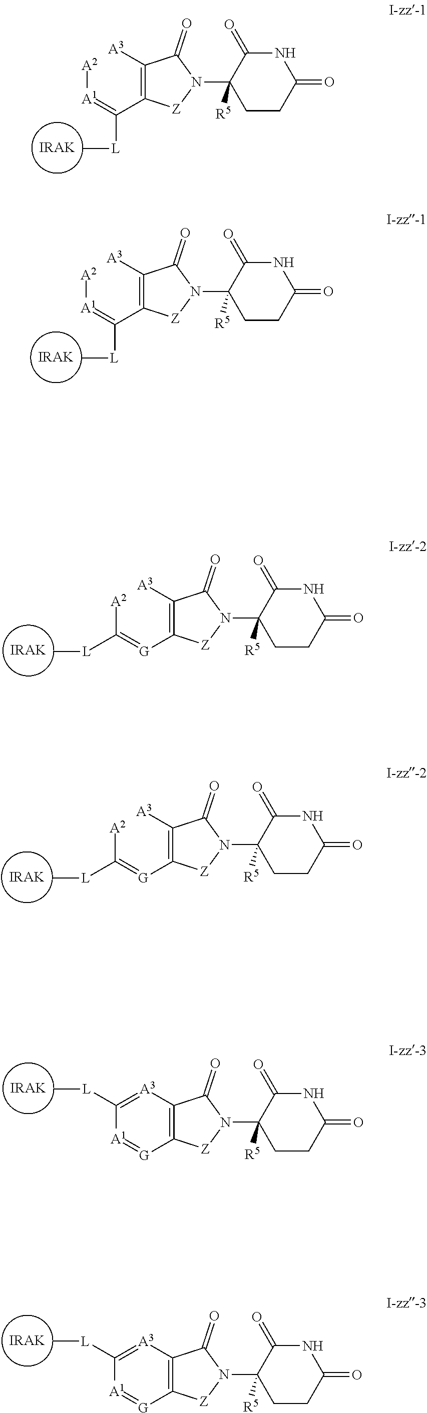

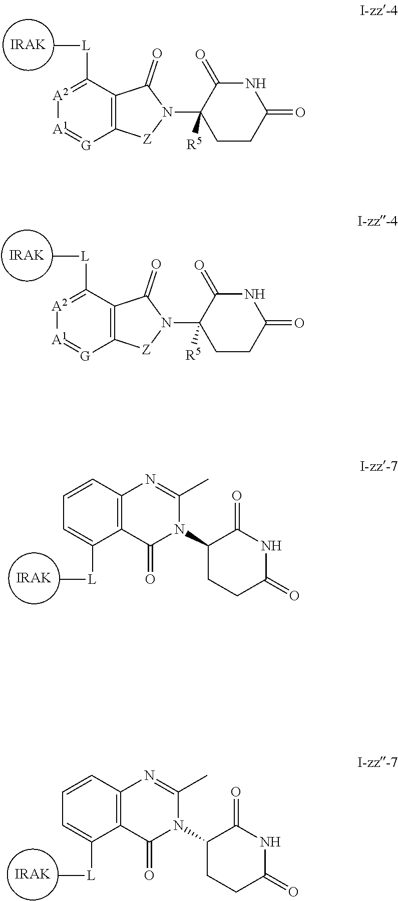

or a pharmaceutically acceptable salt thereof, wherein L and IRAK are as defined above and described in embodiments herein, and wherein each of the variables A¹, A², A³, R⁵, G and Z is as defined and described in WO 2017/176958, the entirety of which is herein incorporated by reference.

In certain embodiments, the present invention provides a compound of Formula I, wherein LBM is a MDM2 (i.e. human double minute 2 or HDM2) E3 ligase binding moiety thereby forming a compound of formula I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, or I-aaa-18 respectively:

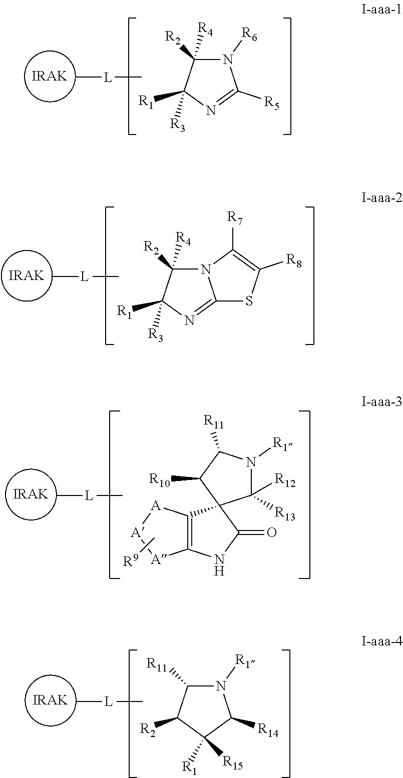

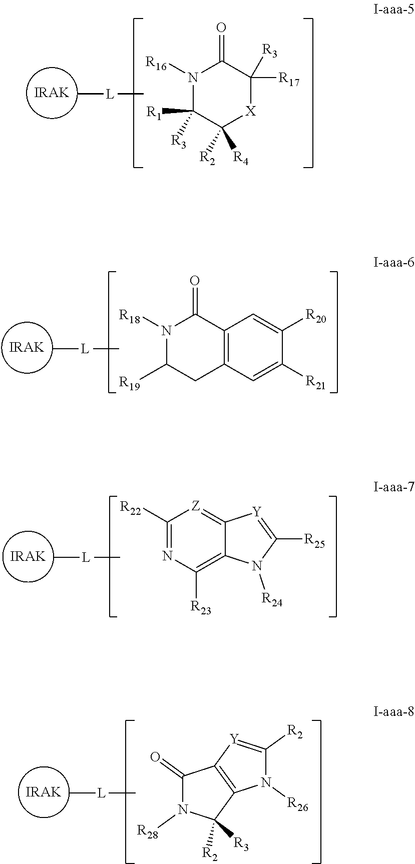

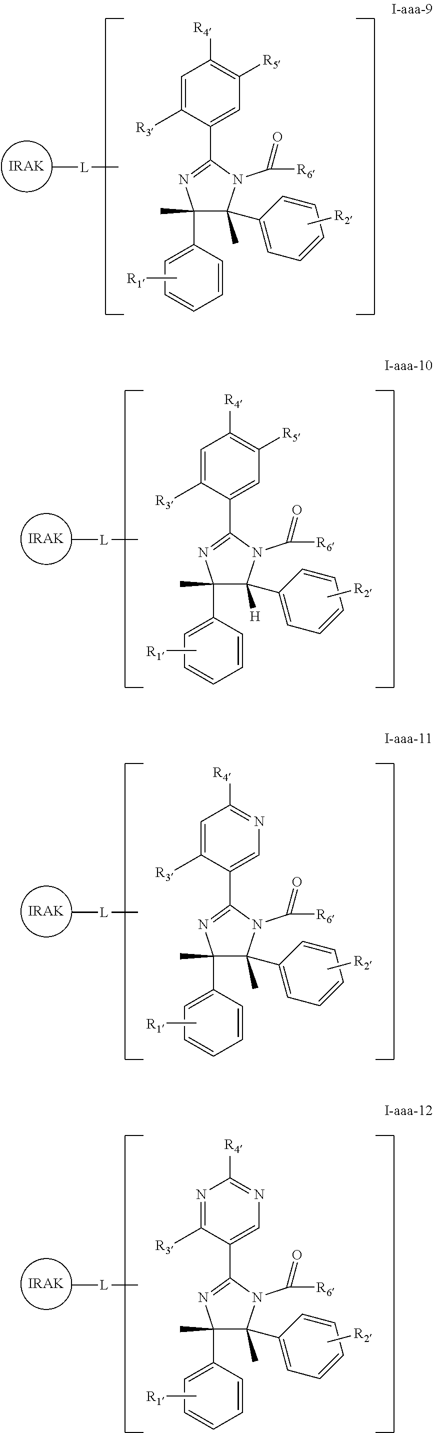

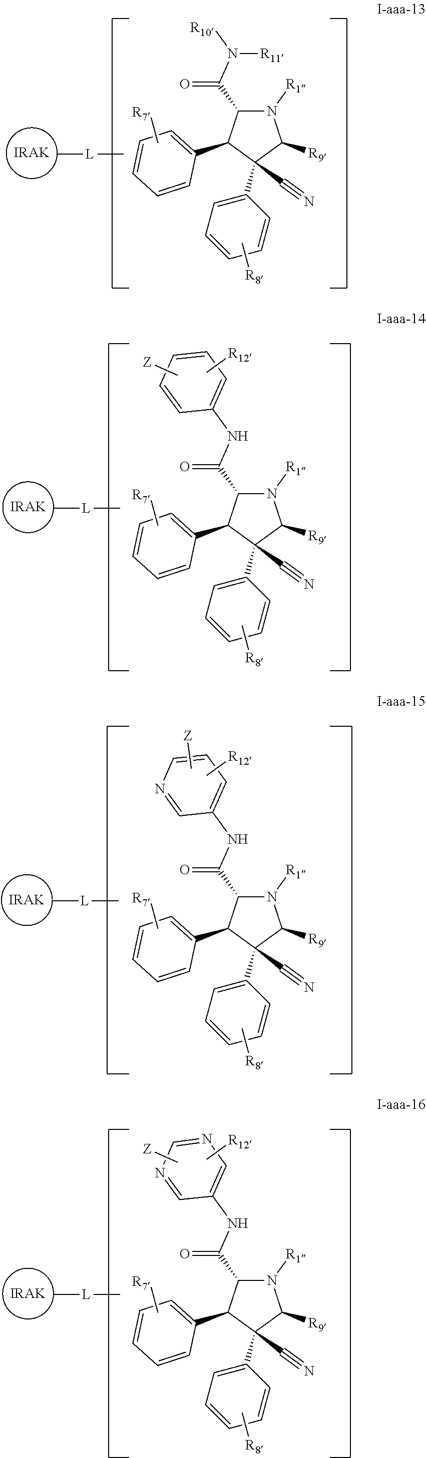

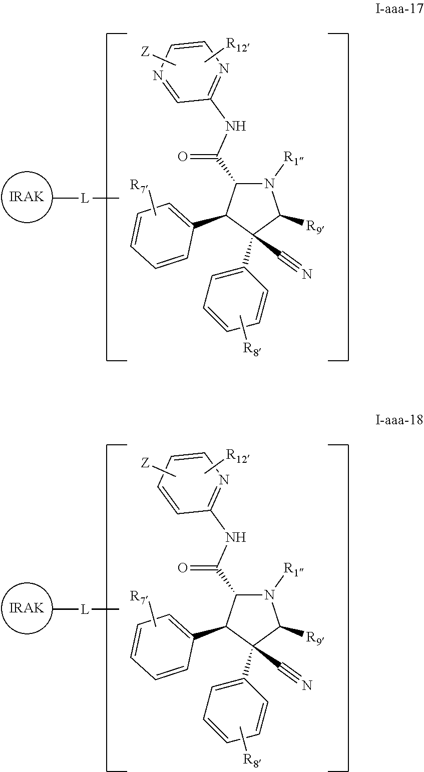

or a pharmaceutically acceptable salt thereof, wherein L and IRAK are as defined above and described in embodiments herein, and wherein each of the variables R₁, R₂, R₃, R₄, R₅, R₆, R₇, R₈, R₉, R₁₀, Rn, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R_{1′}, R_{2′}, R_{3′}, R_{4′}, R_{5′}, R_{6′}, R_{7′}, R_{8′}, R_{9′}, R_{10′}, R_{11′}, R_{12′}, R_{1″}, A, A′, A″, X, Y, and Z is as defined and described in WO 2017/011371 and US 2017/0008904, the entirety of each of which is herein incorporated by reference.

In certain embodiments, the present invention provides a compound of Formula I, wherein LBM is an IAP E3 ubiquitin ligase binding moiety thereby forming a compound of formula I-bbb-1, I-bbb-2, I-bbb-3, or I-bbb-4 respectively:

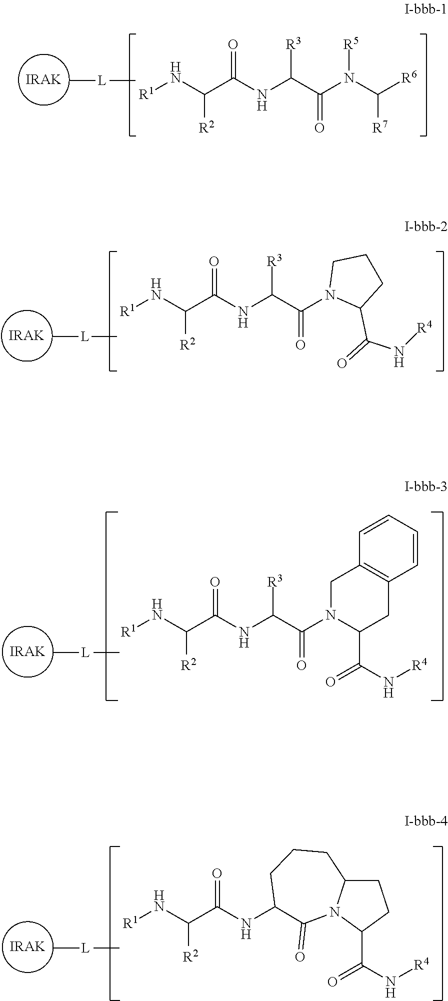

or a pharmaceutically acceptable salt thereof, wherein L and IRAK are as defined above and described in embodiments herein, and wherein each of the variables R¹, R², R³, R⁴, R⁵, R⁶, and R⁷, is as defined and described in WO 2017/011590 and US 2017/0037004, the entirety of each of which is herein incorporated by reference.

In certain embodiments, the present invention provides a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1, I-ccc-2, or I-ccc-3:

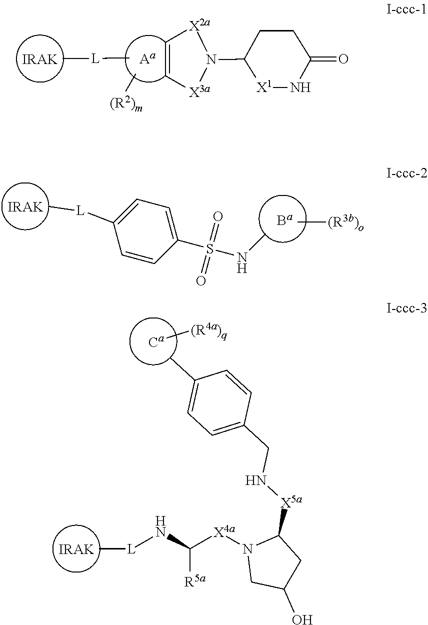

or a pharmaceutically acceptable salt thereof, wherein L and IRAK is as defined above and described in embodiments herein, and wherein:
each of X¹, X^{2a}, and X^{3a} is independently a bivalent moiety selected from a covalent bond, —CH₂—, —C(O)—, —C(S)—, or

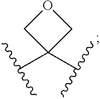 [SMILES: *C1(*)COC1]

each of X^{4a} and X^{5a} is independently a bivalent moiety selected from —CH₂—, —C(O)—, —C(S)—, or

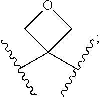 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
o is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-ccc-1, wherein LBM is an E3 ubiquitin ligase (cereblon) binding moiety thereby forming a compound of formula I-ccc′-1 or I-ccc″-1:

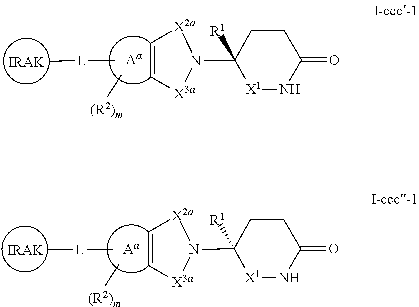

or a pharmaceutically acceptable salt thereof, wherein IRAK, L, Ring A^{a}, X¹, X^{2a}, X^{3a}, R¹, R² and m are as described above.

As defined above and described herein, each of X¹, X^{2a}, and X^{3a} is independently a bivalent moiety selected from a covalent bond, —CH₂—, —C(O)—, —C(S)—, or

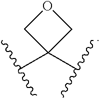 [SMILES: *C1(*)COC1]

In some embodiments, X¹ is a covalent bond, —CH₂—, —C(O)—, —C(S)—, or

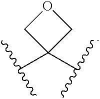 [SMILES: *C1(*)COC1]

In some embodiments, X¹ is selected from those depicted in Table 1, below.

In some embodiments, X^{2a} is a covalent bond, —CH₂—, —C(O)—, —C(S)—, or

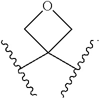 [SMILES: *C1(*)COC1]

In some embodiments, X^{2a} is selected from those depicted in Table 1, below.

In some embodiments, X^{3a} is a covalent bond, —CH₂—, —C(O)—, —C(S)—, or

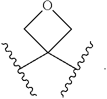 [SMILES: *C1(*)COC1]

In some embodiments, X^{3a} is selected from those depicted in Table 1, below.

As defined above and described herein, each of X⁴ and X⁵ is independently a bivalent moiety selected from —CH₂—, —C(O)—, —C(S)—, or

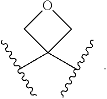 [SMILES: *C1(*)COC1]

In some embodiments, X^{4a} is —CH₂—, —C(O)—, —C(S)—, or

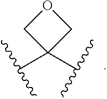 [SMILES: *C1(*)COC1]

In some embodiments, X^{4a} is selected from those depicted in Table 1, below.

In some embodiments, X⁵a is —CH₂—, —C(O)—, —C(S)—, or

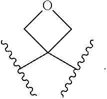 [SMILES: *C1(*)COC1]

In some embodiments, X^{5a} is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{5a} a is hydrogen or C₁₋₆ aliphatic.

In some embodiments, R^{5a} a is t-butyl.

In some embodiments, R^{5a} a is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen or sulfur. In some embodiments Ring A^{a} is a fused 5-membered heteroaryl with 1-3 heteroatoms independently selected from nitrogen, oxygen or sulfur.

In some embodiments, Ring A^{a} is a fused phenyl.

In some embodiments, Ring A^{a} is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, or sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, or sulfur.

In some embodiments, Ring B^{a} is

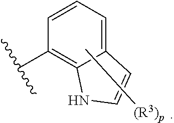

In some embodiments, Ring B^{a} is selected from those depicted in Table 1, below.

As defined above and described herein, Ring C^{a} is selected from 6-membered aryl containing 0-2 nitrogen atoms or a 5-membered heteroaryl with 1-3 heteroatoms independently selected from nitrogen, oxygen or sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen or sulfur.

In some embodiments, Ring C^{a} is

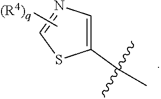

In some embodiments, Ring C^{a} is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of formula I, wherein LBM is a VHL binding moiety thereby forming a compound of formula I-ddd:

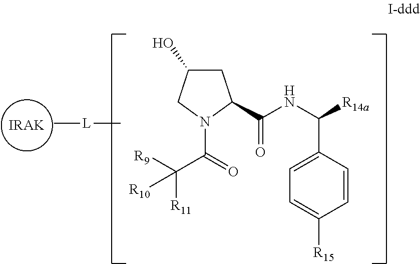

or a pharmaceutically acceptable salt thereof, wherein L and IRAK are as defined above and described in embodiments herein, and wherein each of the variables R₉, R₁₀, R₁₁, R₁₄ₐ, and R₁₅ is as described and defined in WO 2017/030814, WO 2016/118666, and US 2017/0327469, the entirety of each of which is herein incorporated by reference.

In certain embodiments, the present invention provides a compound of formula I, wherein LBM is a VHL binding moiety thereby forming a compound of formula I-eee-1 or I-eee-2:

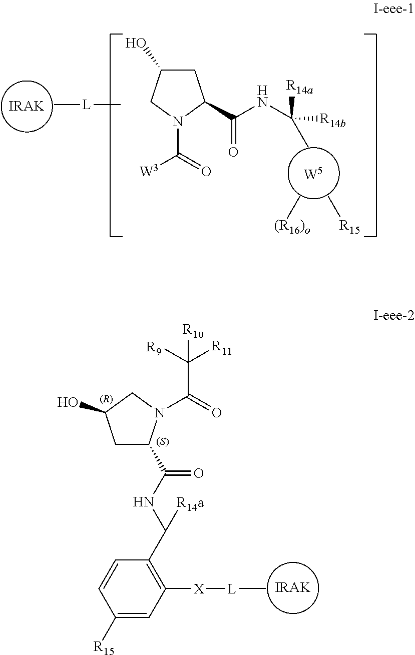

or a pharmaceutically acceptable salt thereof wherein L and IRAK are as defined above and described in embodiments herein, and wherein each of the variables X, W, R₉, R₁₀, R₁₁, R₁₄ₐ, and R_{14b}, R₁₅, R¹⁶, and o is as described and defined in WO 2017/030814, WO 2016/118666, and US 2017/0327469, the entirety of each of which is herein incorporated by reference.

In certain embodiments, the present invention provides a compound of formula I, wherein LBM is an IAP binding moiety thereby forming a compound of formula I-fff:

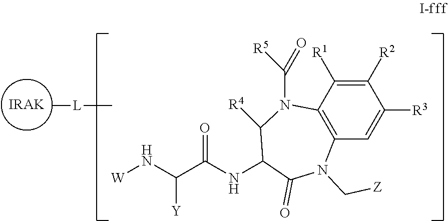

or a pharmaceutically acceptable salt thereof, wherein L and IRAK are as defined above and described in embodiments herein, and wherein each of the variables W, Y, Z, R¹, R², R³, R⁴, and R⁵ is as described and defined in WO 2014/044622, US 2015/0225449. WO 2015/071393, and US 2016/0272596, the entirety of each of which is herein incorporated by reference.

In certain embodiments, the present invention provides a compound of formula I, wherein LBM is a MDM2 binding moiety thereby forming a compound of formula I-ggg:

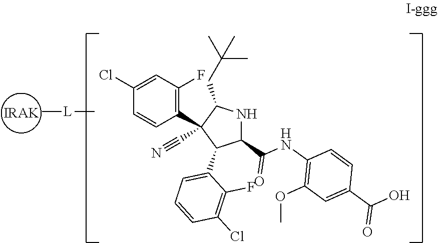

or a pharmaceutically acceptable salt thereof, wherein L and IRAK are as defined above and described in embodiments herein, as described and defined in Hines, J. et al., Cancer Res. (DOI: 10.1158/0008-5472.CAN-18-2918), the entirety of each of which is herein incorporated by reference.

In certain embodiments, the present invention provides a compound of formula I, wherein LBM is a DCAF16 binding moiety thereby forming a compound of formula I-hhh:

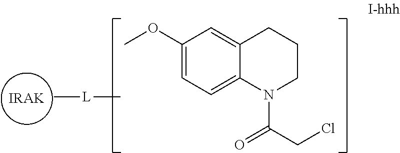

or a pharmaceutically acceptable salt thereof, wherein L and IRAK are as defined above and described in embodiments herein, as described and defined in Zhang, X. et al., bioRxiv (doi: https://doi.org/10.1101/443804), the entirety of each of which is herein incorporated by reference.

In certain embodiments, the present invention provides a compound of formula I, wherein LBM is a RNF114 binding moiety thereby forming a compound of formula I-iii:

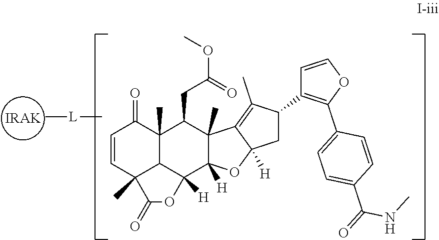

or a pharmaceutically acceptable salt thereof, wherein L and IRAK are as defined above and described in embodiments herein, as described and defined in Spradin, J. N. et al., bioRxiv (doi: https://doi.org/10.1101/436998), the entirety of each of which is herein incorporated by reference.

In certain embodiments, the present invention provides a compound of formula I, wherein LBM is a RNF4 binding moiety thereby forming a compound of formula I-jjj:

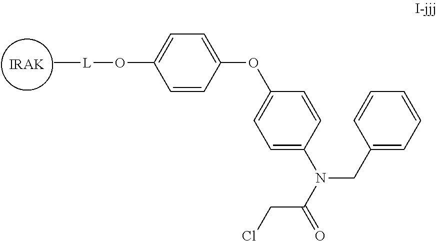

or a pharmaceutically acceptable salt thereof, wherein L and IRAK are as defined above and described in embodiments herein, as described and defined in Ward, C. C., et al., bioRxiv (doli:tps//doi org/10.1101/439125), the entirety of each of which is herein incorporated by reference.

In certain embodiments, the present invention provides a compound of formula I, wherein LBM is a VHL binding moiety thereby forming a compound of formula I-nnn-1 or I-nnn-2:

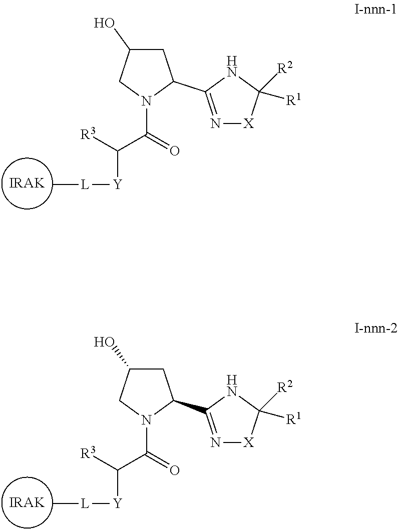

or a pharmaceutically acceptable salt thereof, wherein L and IRAK are as defined above and described in embodiments herein, and wherein each of the variables R¹, R², R³, X, and Y is as defined and described in WO 2019/084026, the entirety of each of which is herein incorporated by reference.

In certain embodiments, the present invention provides a compound of formula I, wherein LBM is a VHL binding moiety thereby forming a compound of formula I-ooo-1 or I-ooo-2:

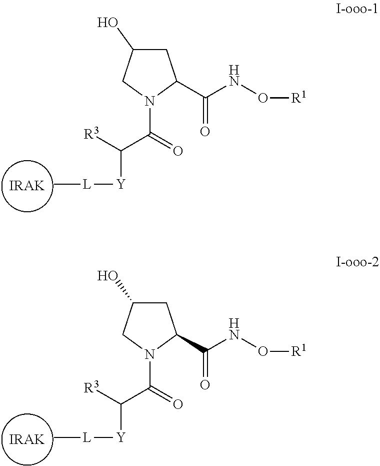

or a pharmaceutically acceptable salt thereof, wherein L and IRAK are as defined above and described in embodiments herein, and wherein each of the variables R¹, R³, and Y is as defined and described in WO 2019/084030, the entirety of each of which is herein incorporated by reference.

In certain embodiments, the present invention provides a compound of formula I, wherein LBM is a E3 ubiquitin ligase (cereblon) binding moiety thereby forming a compound of formula I-ppp-1, I-ppp-2, I-ppp-3, or I-ppp-4:

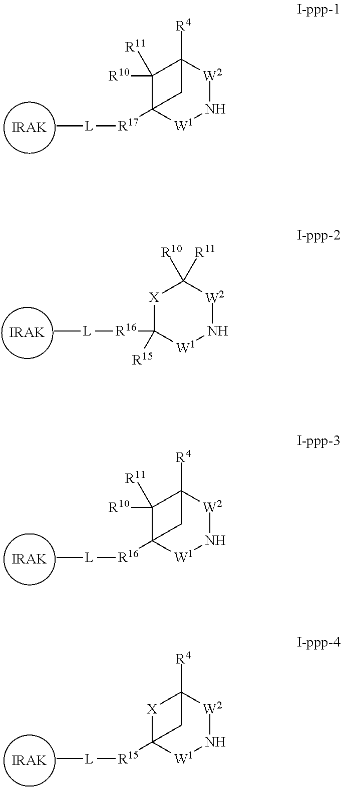

or a pharmaceutically acceptable salt thereof, wherein L and IRAK are as defined above and described herein, and wherein each of the variables R⁴, R¹⁰, R¹¹, R¹⁵, R¹⁶, R¹⁷, W¹, W², and X is as defined in WO 2019/099868 which is herein incorporated by reference in its entirety, and wherein

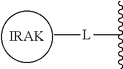

is attached to R¹⁷ or R¹⁶ at the site of attachment of R¹² as defined in WO 2018/237026, such that

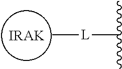

takes the place of the R¹² substituent.

In some embodiments, LBM is

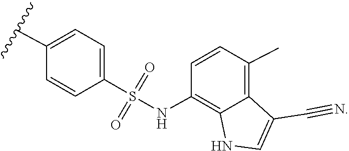 [SMILES: *c1ccc(S(=O)(=O)Nc2ccc(C)c3c(C#N)c[nH]c23)cc1]

In some embodiments, LBM is

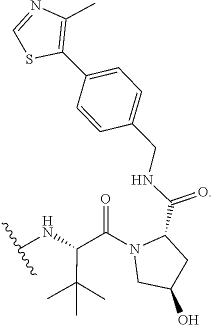 [SMILES: *N[C@H](C(=O)N1C[C@H](O)C[C@H]1C(=O)NCc1ccc(-c2scnc2C)cc1)C(C)(C)C]

In some embodiments, LBM is

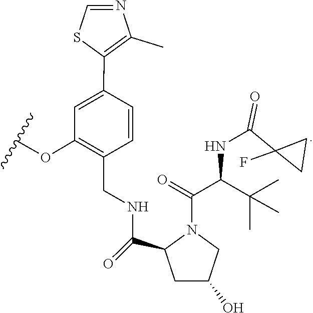 [SMILES: *Oc1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

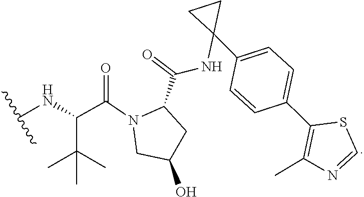 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

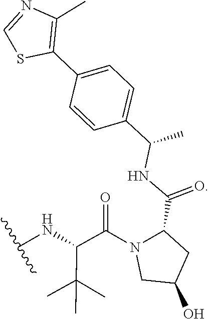 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

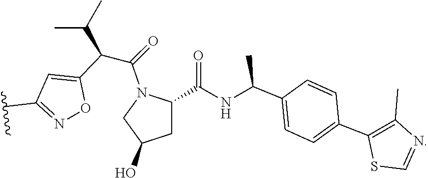 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

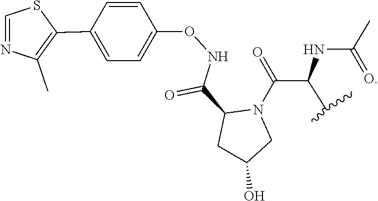 [SMILES: *[C@H](NC(C)=O)C(=O)N1C[C@H](O)C[C@H]1C(=O)NOc1ccc(-c2scnc2C)cc1]

In some embodiments, LBM is

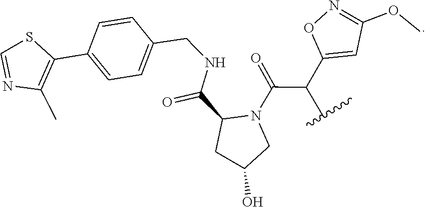 [SMILES: *C(C(=O)N1C[C@H](O)C[C@H]1C(=O)NCc1ccc(-c2scnc2C)cc1)c1cc(OC)no1]

In some embodiments, LBM is

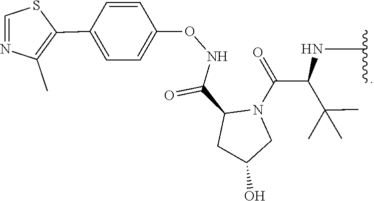 [SMILES: *N[C@H](C(=O)N1C[C@H](O)C[C@H]1C(=O)NOc1ccc(-c2scnc2C)cc1)C(C)(C)C]

In some embodiments, LBM is

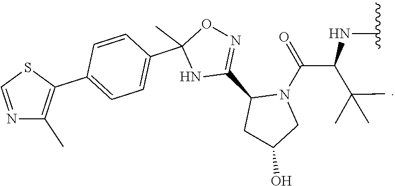 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

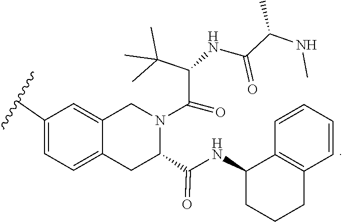 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

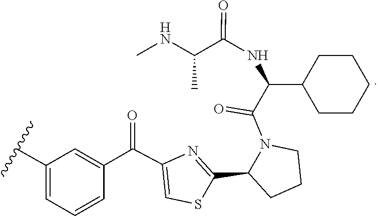 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

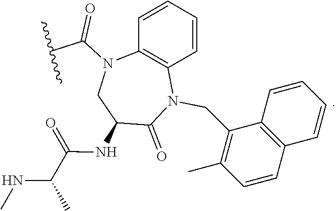 [SMILES: *C(=O)N1C[C@H](NC(=O)[C@H](C)NC)C(=O)N(Cc2c(C)ccc3ccccc23)c2ccccc21]

In some embodiments, LBM is

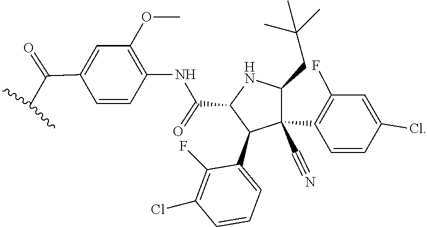 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

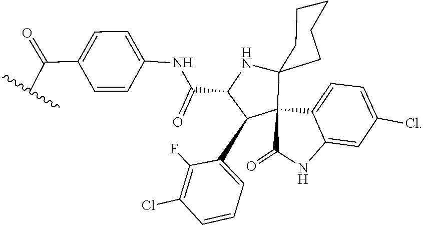 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

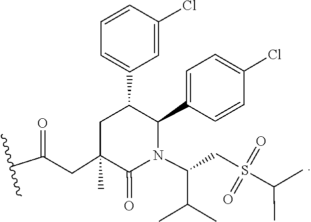 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

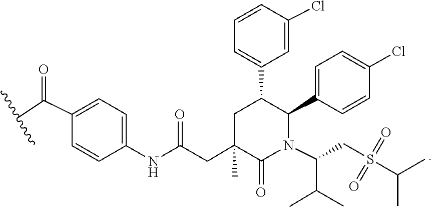 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

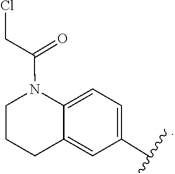 [SMILES: *c1ccc2c(c1)CCCN2C(=O)CCl]

In some embodiments, LBM is

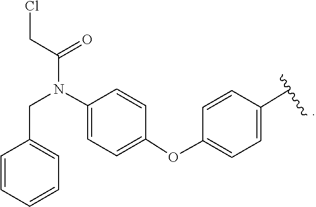 [SMILES: *c1ccc(Oc2ccc(N(Cc3ccccc3)C(=O)CCl)cc2)cc1]

In some embodiments, LBM is

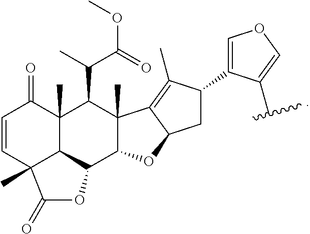 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

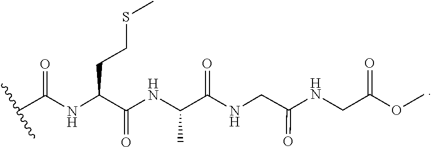 [SMILES: *C(=O)N[C@@H](CCSC)C(=O)N[C@@H](C)C(=O)NCC(=O)NCC(=O)OC]

In some embodiments, LBM is

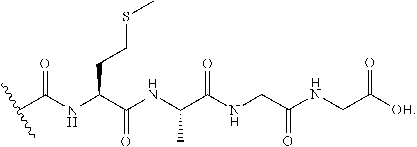 [SMILES: *C(=O)N[C@@H](CCSC)C(=O)N[C@@H](C)C(=O)NCC(=O)NCC(=O)O]

In some embodiments, LBM is

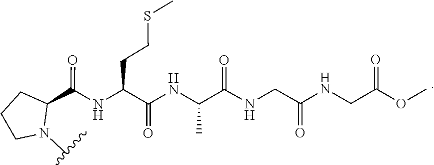 [SMILES: *N1CCC[C@H]1C(=O)N[C@@H](CCSC)C(=O)N[C@@H](C)C(=O)NCC(=O)NCC(=O)OC]

In some embodiments, LBM is

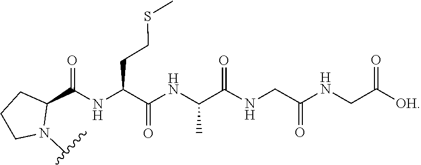 [SMILES: *N1CCC[C@H]1C(=O)N[C@@H](CCSC)C(=O)N[C@@H](C)C(=O)NCC(=O)NCC(=O)O]

In some embodiments, LBM is

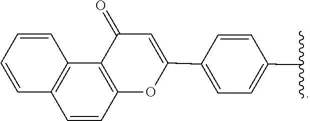 [SMILES: *c1ccc(-c2cc(=O)c3c(ccc4ccccc43)o2)cc1]

In some embodiments, LBM is

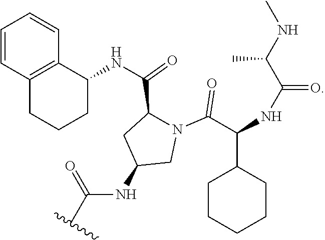 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

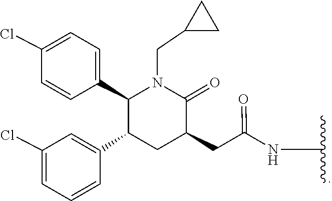 [SMILES: *NC(=O)C[C@H]1C[C@H](c2cccc(Cl)c2)[C@@H](c2ccc(Cl)cc2)N(CC2CC2)C1=O]

In some embodiments, LBM is

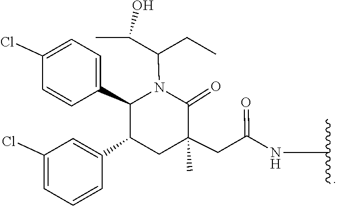 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

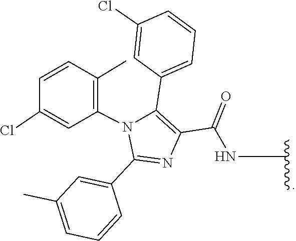 [SMILES: *NC(=O)c1nc(-c2cccc(C)c2)n(-c2cc(Cl)ccc2C)c1-c1cccc(Cl)c1]

In some embodiments, LBM

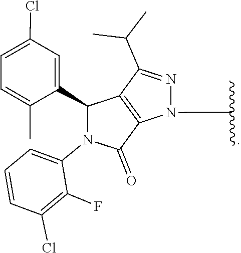 [SMILES: *n1nc(C(C)C)c2c1C(=O)N(c1cccc(Cl)c1F)[C@H]2c1cc(Cl)ccc1C]

In certain embodiments, the present invention provides a compound of formula I, wherein LBM is a CRBN E3 ubiquitin ligase binding moiety thereby forming a compound of formula I-qqq:

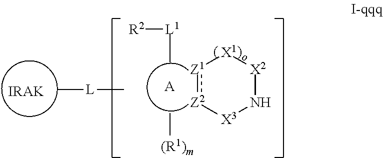

or a pharmaceutically acceptable salt thereof, wherein L and IRAK are as defined above and described in embodiments herein, wherein:
each X¹ is independently —CH₂—, —O—, —NR—, —CF₂—,

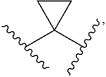 [SMILES: *C1(*)CC1]

### —C(O)—, —C(S)—, or

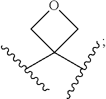 [SMILES: *C1(*)COC1]

X² and X³ are independently —CH₂—, —C(O)—, —C(S)—, or

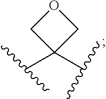 [SMILES: *C1(*)COC1]

Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R², and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

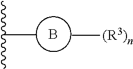

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
o is 0, 1, or 2.

As defined above and described herein each X¹ is independently a covalent bond, —CH₂—, —O—, —NR—, —CF₂—,

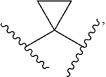 [SMILES: *C1(*)CC1]

### —C(O)—, —C(S)—, or

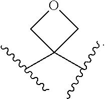 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

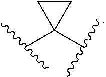 [SMILES: *C1(*)CC1]

In some embodiments, X¹ is —C(O)—. In some embodiments, X¹ is —C(S)—. In some embodiments, X¹ is

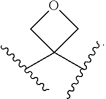 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

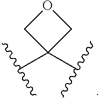 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

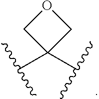 [SMILES: *C1(*)COC1]

In certain embodiments, X² and X³ are independently selected from those shown in the compounds of Table 1.

As defined above and described herein, X⁴ is a covalent bond, —CH₂—, —CR₂—, —O—, —NR—, —CF₂—,

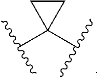 [SMILES: *C1(*)CC1]

### —C(O)—, —C(S)—, or

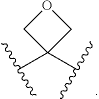 [SMILES: *C1(*)COC1]

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In certain embodiments, Ring A is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R². In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, or sulfur.

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

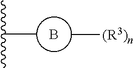

or hydrogen.

In some embodiment R² is

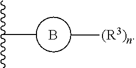

In some embodiments, R² is hydrogen.

In certain embodiments, R² is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring B is selected from those shown in the compounds of Table 1.

As defined above and described herein, each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1.

As defined above and described herein, o is 0, 1, or 2.

In some embodiments, n is 0. In some embodiments, n is 1. In some embodiments, m is 2.

In certain embodiments, o is selected from those shown in the compounds of Table 1.

In some embodiments, the present invention provides a compound of formula I-qqq, wherein Ring A is benzo, o is 1, X¹ is —CH₂—, X² and X³ are —C(O)—, and Z¹ and Z² are carbon atoms as shown, to provide a compound of formula I-qqq-1:

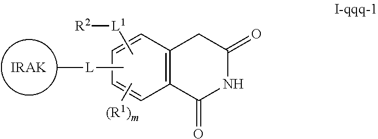

or a pharmaceutically acceptable salt thereof, wherein each of IRAK, L, L¹, R¹, R², and m is as defined above and described in embodiments herein, both singly and in combination.

In some embodiments, the present invention provides a compound of formula I-qqq, wherein Ring A is benzo, o is 1, X¹, X² and X³ are —C(O)—, and Z¹ and Z² are carbon atoms as shown, to provide a compound of formula I-qqq-12:

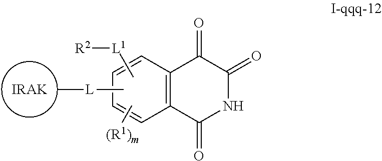

or a pharmaceutically acceptable salt thereof, wherein each of IRAK, L, L¹, R¹, R², and m is as defined above and described in embodiments herein, both singly and in combination.

In some embodiments, LBM is

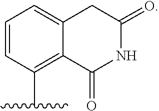 [SMILES: *c1cccc2c1C(=O)NC(=O)C2]

In some embodiments, LBM is

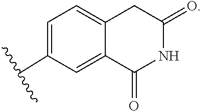 [SMILES: *c1ccc2c(c1)C(=O)NC(=O)C2]

In some embodiments, LBM

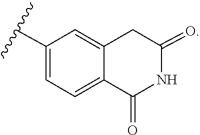 [SMILES: *c1ccc2c(c1)CC(=O)NC2=O]

In some embodiments, LBM is

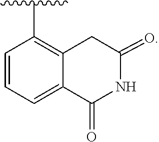 [SMILES: *c1cccc2c1CC(=O)NC2=O]

In some embodiments, LBM is

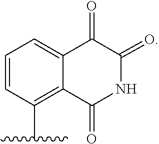 [SMILES: *c1cccc2c1C(=O)NC(=O)C2=O]

In some embodiments, LBM is

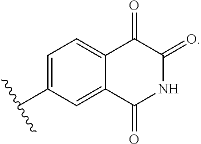 [SMILES: *c1ccc2c(c1)C(=O)NC(=O)C2=O]

In some embodiments, LBM is

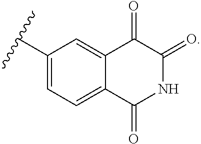 [SMILES: *c1ccc2c(c1)C(=O)C(=O)NC2=O]

In some embodiments, LBM is

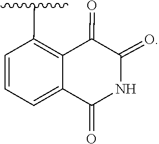 [SMILES: *c1cccc2c1C(=O)C(=O)NC2=O]

In some embodiments, LBM is selected from those in Table 1, below.

In certain embodiments, the present invention provides a compound of formula I, wherein LBM is a RPN13 binding moiety thereby forming a compound of formula I-rrr:

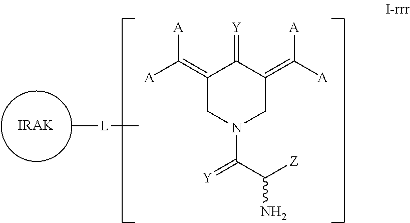

or a pharmaceutically acceptable salt thereof, wherein L and IRAK are as defined above and described in embodiments herein, and wherein each of the variables A, Y, and Z is as described and defined in WO 2019/165229, the entirety of each of which is herein incorporated by reference.

In certain embodiments, the present invention provides a compound of formula I, wherein LBM is a Ubr1 binding moiety as described in Shanmugasundaram, K. et al, J. Bio. Chem. 2019, doi: 10.1074/jbc.AC119.010790, the entirety of each of which is herein incorporated by reference, thereby forming a compound of formula I-sss-1 or I-sss-2:

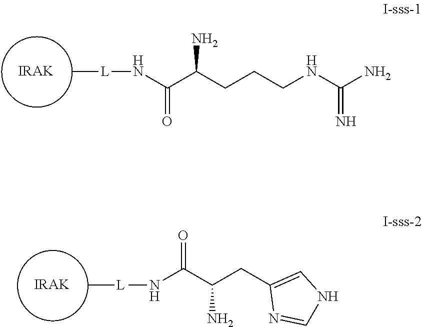

or a pharmaceutically acceptable salt thereof, wherein L and IRAK are as defined above and described in embodiments herein.

In certain embodiments, the present invention provides a compound of formula I, wherein LBM is a CRBN E3 ubiquitin ligase binding moiety thereby forming a compound of formula I-uuu-1, I-uuu-2, I-uuu-3, or I-uuu-4:

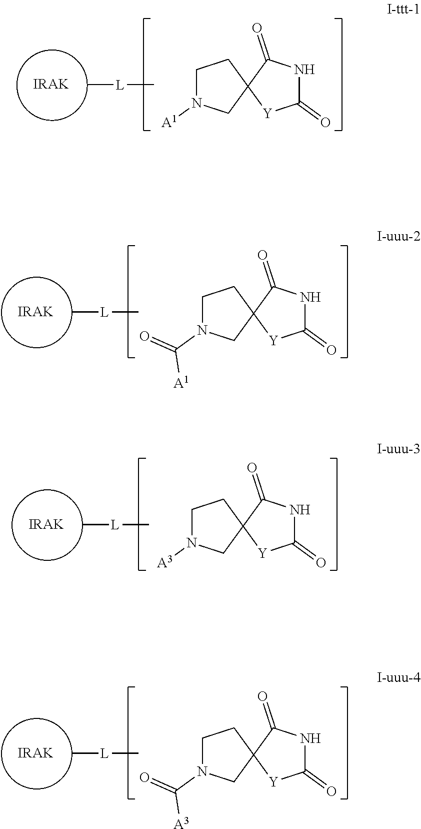

or a pharmaceutically acceptable salt thereof, wherein L and IRAK are as defined above and described in embodiments herein, and wherein each of the variables Y, A¹, and A³ is as described and defined in WO 2019/236483, the entirety of each of which is herein incorporated by reference.

In certain embodiments, the present invention provides a compound of formula I, wherein LBM is human kelch-like ECH-associated protein 1 (KEAP1) thereby forming a compound of formula I-vvv:

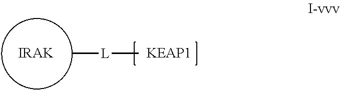

or a pharmaceutically acceptable salt thereof, wherein L and IRAK are as defined above and described in embodiments herein, both singly and in combination.

In certain embodiments, the present invention provides a compound of formula I, wherein LBM is KEAP1 binding moiety as recited in Lu et al., Euro. J. Med. Chem., 2018, 146:251-9, thereby forming a compound of formula I-www:

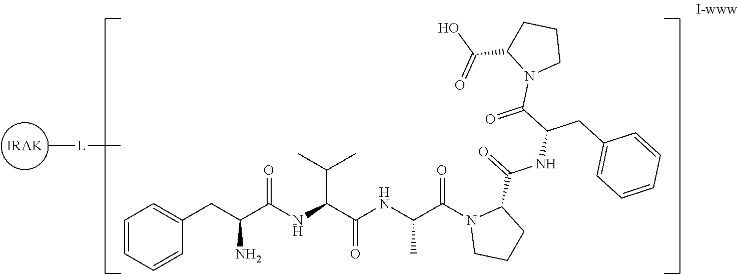

or a pharmaceutically acceptable salt thereof, wherein L and IRAK are as defined above and described in embodiments herein, both singly and in combination.

In certain embodiments, the present invention provides a compound of formula I, wherein LBM is KEAP1-NRF2 binding moiety thereby forming a compound of formula I-xxx or I-xxx-2:

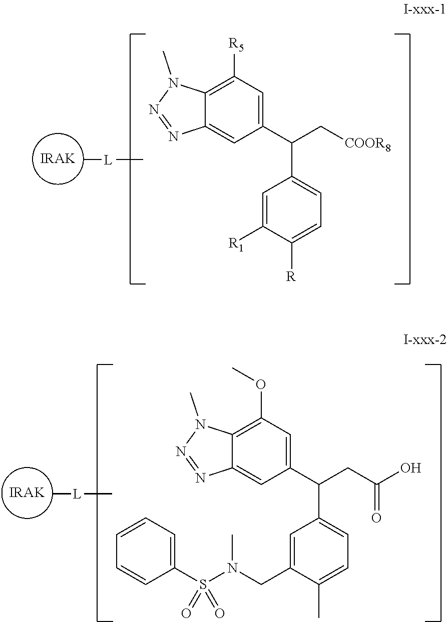

or a pharmaceutically acceptable salt thereof, wherein L and IRAK are as defined above and described in embodiments herein, and wherein each of the variables R, R₁, R₅, and R₈ is as described and defined in WO 2020/018788, the entirety of each of which is herein incorporated by reference.

In certain embodiments, the present invention provides a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-yyy-1 or I-yyy-2:

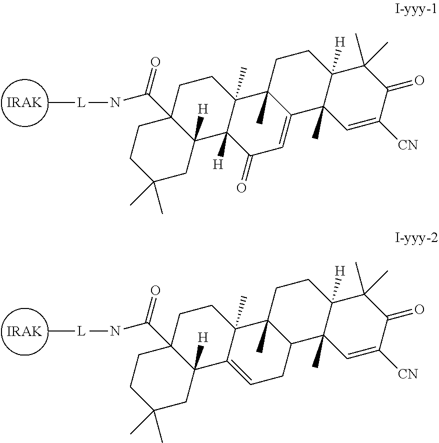

or a pharmaceutically acceptable salt thereof, wherein L and IRAK are as defined above and described in embodiments herein, both singly and in combination.

In some embodiments, the present invention provides a compound of formula I-a, wherein LBM is RNF114 E3 ubiquitin ligase

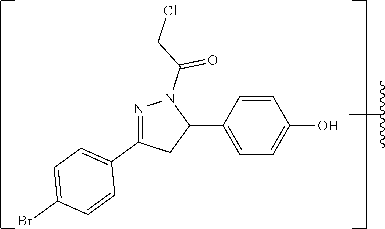

thereby forming a compound of formula I-zzz-1:

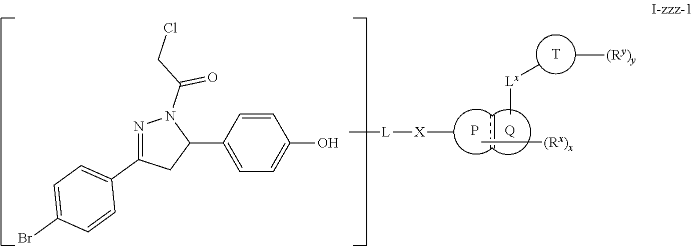

or a pharmaceutically acceptable salt thereof, wherein each of L, L^{x}, X, Ring P, Ring Q, Ring T, R^{x}, R^{y}, x, and y is as defined above and described in embodiments herein, both singly and in combination.

In some embodiments, the present invention provides a compound of formula I-a, wherein LBM is DCAF15 E3 ubiquitin ligase

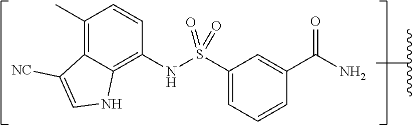

thereby forming a compound of formula I-zzz-2:

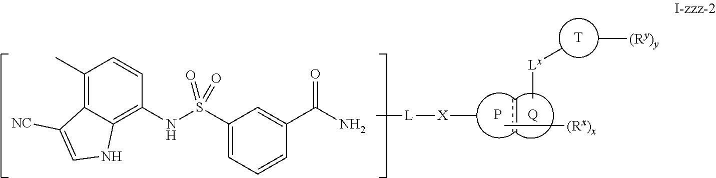

or a pharmaceutically acceptable salt thereof, wherein each of L, L^{x}, X, Ring P, Ring Q, Ring T, R^{x}, R^{y}, x, and y is as defined above and described in embodiments herein, both singly and in combination.

In some embodiments, LBM is Br

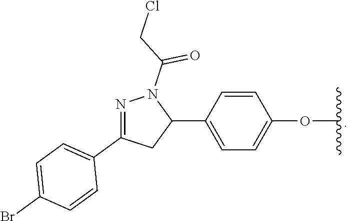 [SMILES: *Oc1ccc(C2CC(c3ccc(Br)cc3)=NN2C(=O)CCl)cc1]

In some embodiments, LBM is

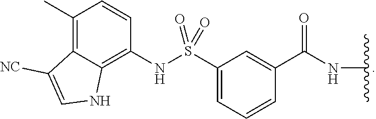 [SMILES: *NC(=O)c1cccc(S(=O)(=O)Nc2ccc(C)c3c(C#N)c[nH]c23)c1]

### Lysine Mimetic

In some embodiments, DIM is LBM as described above and herein. In some embodiments, DIM is lysine mimetic. In some embodiments, the covalent attachment of ubiquitin to a member of the IRAK kinase family (i.e., IRAK-1, -2, -3, or -4) is achieved through the action of a lysine mimetic. In some embodiments, upon the binding of a compound of formula I to IRAK-1, the moiety that mimics a lysine undergoes ubiquitination thereby marking IRAK-1 for degradation via the Ubiquitin-Proteasome Pathway (UPP). In some embodiments, upon the binding of a compound of formula I to IRAK-2, the moiety that mimics a lysine undergoes ubiquitination thereby marking IRAK-2 for degradation via the Ubiquitin-Proteasome Pathway (UPP). In some embodiments, upon the binding of a compound of formula I to IRAK-3, the moiety that mimics a lysine undergoes ubiquitination thereby marking IRAK-3 for degradation via the Ubiquitin-Proteasome Pathway (UPP). In some embodiments, upon the binding of a compound of formula I to IRAK-4, the moiety that mimics a lysine undergoes ubiquitination thereby marking IRAK-4 for degradation via the Ubiquitin-Proteasome Pathway (UPP).

In some embodiments, DIM is

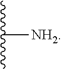 [SMILES: *N]

In some embodiments, DIM is

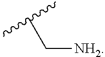 [SMILES: *CN]

In some embodiments, DIM is

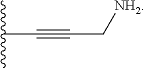 [SMILES: *C#CCN]

In some embodiments, DIM is selected from those depicted in Table 1, below.

In some embodiments, the present invention provides the compound of formula I wherein DIM is

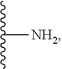 [SMILES: *N]

thereby forming a compound of formula I-kkk-1:

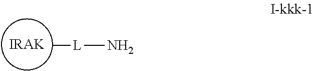

or a pharmaceutically acceptable salt thereof, wherein each of IRAK and L is as defined above and described in embodiments herein, both singly and in combination.

In some embodiments, the present invention provides the compound of formula I wherein DIM is

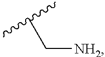 [SMILES: *CN]

thereby forming a compound of formula I-kkk-2:

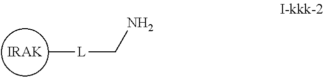

or a pharmaceutically acceptable salt thereof, wherein each of IRAK and L is as defined above and described in embodiments herein, both singly and in combination.

In some embodiments, the present invention provides the compound of formula I wherein DIM is

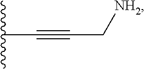 [SMILES: *C#CCN]

thereby forming a compound of formula I-kkk-3:

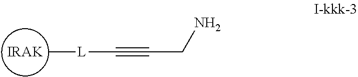

or a pharmaceutically acceptable salt thereof, wherein each of IRAK and L is as defined above and described in embodiments herein, both singly and in combination.

In certain embodiments, the present invention provides a compound of Formula I, wherein DIM is lysine mimetic or

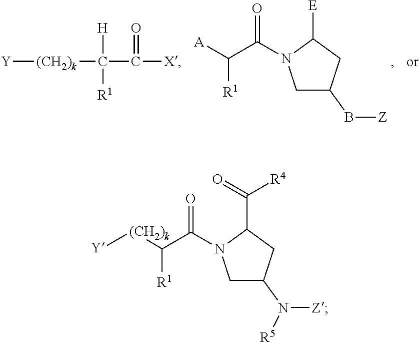

thereby forming a compound of formulae I-lll-1, I-lll-2, or I-lll-3, respectively:

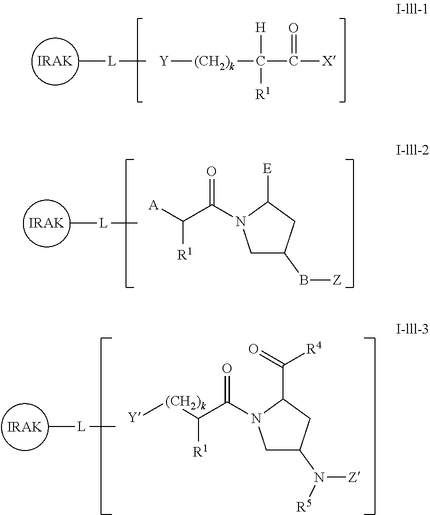

or a pharmaceutically acceptable salt thereof, wherein L and IRAK are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

### Hydrogen Atom

In some embodiments, DIM is a hydrogen atom. In some embodiments, the covalent attachment of ubiquitin to one or more members of the IRAK kinase family (i.e., IRAK-1, -2, -3, or -4) is achieved through a provided compound wherein DIM is a hydrogen atom. In some embodiments, upon the binding of a compound of formula I to IRAK-1, the DIM moiety being hydrogen effectuates ubiquitination thereby marking IRAK-1 for degradation via the Ubiquitin-Proteasome Pathway (UPP). In some embodiments, upon the binding of a compound of formula I to IRAK-2, the DIM moiety being hydrogen effectuates ubiquitination thereby marking IRAK-2 for degradation via the Ubiquitin-Proteasome Pathway (UPP). In some embodiments, upon the binding of a compound of formula I to IRAK-3, the DIM moiety being hydrogen effectuates ubiquitination thereby marking IRAK-3 for degradation via the Ubiquitin-Proteasome Pathway (UPP). In some embodiments, upon the binding of a compound of formula I to IRAK-4, the DIM moiety being hydrogen effectuates ubiquitination thereby marking IRAK-4 for degradation via the Ubiquitin-Proteasome Pathway (UPP).

In some embodiments, DIM is selected from those depicted in Table 1, below.

In some embodiments, the present invention provides the compound of formula I wherein DIM is a hydrogen atom, thereby forming a compound of formula I-mmm:

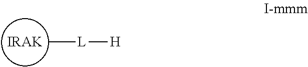

or a pharmaceutically acceptable salt thereof, wherein each of IRAK and L is as defined above and described in embodiments herein, both singly and in combination.

### Linker (L)

As defined above and described herein, L is a bivalent moiety that connects IRAK to LBM or IRAK to DIM.

In some embodiments, L is a bivalent moiety that connects IRAK to LBM. In some embodiments, L is a bivalent moiety that connects IRAK to DIM. In some embodiments, L is a bivalent moiety that connects IRAK to a lysine mimetic.

In some embodiments, L is a covalent bond or a bivalent, saturated or unsaturated, straight or branched C₁₋₅₀ hydrocarbon chain, wherein 0-6 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

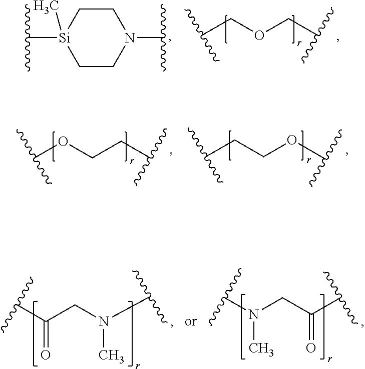

wherein: each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur, wherein r is 0, 1, 2, 3, 4, 5, 6, 7, 8, 9, or 10, and wherein R is as described above (e.g., in formula I-a).

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 4-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 4-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, or sulfur.

In some embodiments, -Cy- is

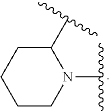 [SMILES: *C1CCCCN1*]

In some embodiments, -Cy- is

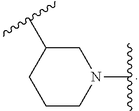 [SMILES: *C1CCCN(*)C1]

In some embodiments, -Cy- is

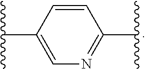 [SMILES: *c1ccc(*)nc1]

In some embodiments, -Cy- is

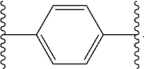 [SMILES: *c1ccc(*)cc1]

In some embodiments, -Cy- is

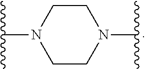 [SMILES: *N1CCN(*)CC1]

In some embodiments, -Cy- is

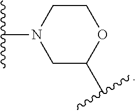 [SMILES: *C1CN(*)CCO1]

In some embodiments, -Cy- is

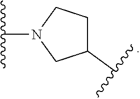 [SMILES: *C1CCN(*)C1]

In some embodiments, -Cy- is

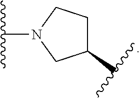 [SMILES: *[C@@H]1CCN(*)C1]

In some embodiments, -Cy- is

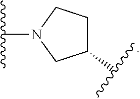 [SMILES: *[C@H]1CCN(*)C1]

In some embodiments, -Cy- is

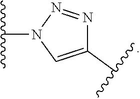 [SMILES: *c1cn(*)nn1]

In some embodiments, -Cy- is

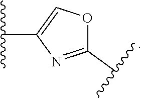 [SMILES: *c1coc(*)n1]

In some embodiments, -Cy- is

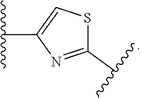 [SMILES: *c1csc(*)n1]

In some embodiments, -Cy- is

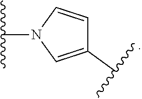 [SMILES: *c1ccn(*)c1]

In some embodiments, -Cy- is

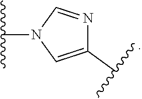 [SMILES: *c1cn(*)cn1]

In some embodiments, -Cy- is

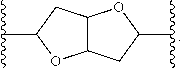 [SMILES: *C1CC2OC(*)CC2O1]

In some embodiments, -Cy- is

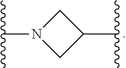 [SMILES: *C1CN(*)C1]

In some embodiments, -Cy- is

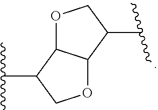 [SMILES: *C1COC2C(*)COC12]

In some embodiments, -Cy- is

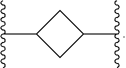 [SMILES: *C1CC(*)C1]

In some embodiments, -Cy- is

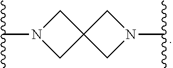 [SMILES: *N1CC2(C1)CN(*)C2]

In some embodiments, -Cy- is

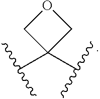 [SMILES: *C1(*)COC1]

In some embodiments, -Cy- is

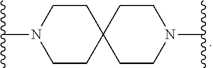 [SMILES: *N1CCC2(CC1)CCN(*)CC2]

In some embodiments, -Cy- is

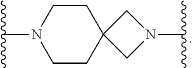 [SMILES: *N1CCC2(CC1)CN(*)C2]

In some embodiments, -Cy- is

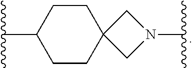 [SMILES: *C1CCC2(CC1)CN(*)C2]

In some embodiments, -Cy- is

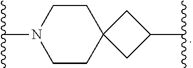 [SMILES: *C1CC2(CCN(*)CC2)C1]

In some embodiments, -Cy- is

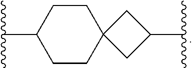 [SMILES: *C1CCC2(CC1)CC(*)C2]

In some embodiments, -Cy- is

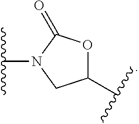 [SMILES: *C1CN(*)C(=O)O1]

In some embodiments, -Cy- is

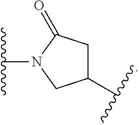 [SMILES: *C1CC(=O)N(*)C1]

In some embodiments, -Cy- is

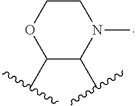 [SMILES: *C1OCCN(C)C1*]

In some embodiments, -Cy- is

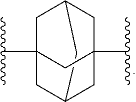 [SMILES: *C12CC3CC(C1)CC(*)(C3)C2]

In some embodiments, -Cy- is

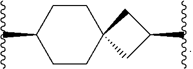

In some embodiments, -Cy- is

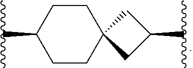

In some embodiments, -Cy- is

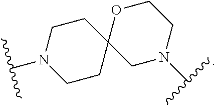 [SMILES: *N1CCC2(CC1)CN(*)CCO2]

In some embodiments, -Cy- is

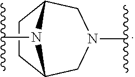 [SMILES: *N1C[C@H]2CC[C@@H](C1)N2*]

In some embodiments, -Cy- is

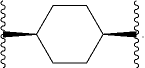 [SMILES: *[C@H]1CC[C@@H](*)CC1]

In some embodiments, -Cy- is

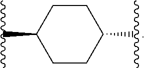 [SMILES: *[C@H]1CC[C@H](*)CC1]

In some embodiments, -Cy- is

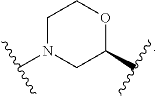 [SMILES: *[C@H]1CN(*)CCO1]

In some embodiments, -Cy- is

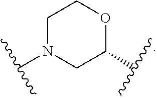 [SMILES: *[C@@H]1CN(*)CCO1]

In some embodiments, -Cy- is

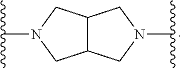 [SMILES: *N1CC2CN(*)CC2C1]

In some embodiments, -Cy- is

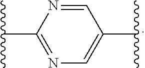 [SMILES: *c1cnc(*)nc1]

In some embodiments, -Cy- is

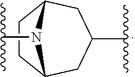 [SMILES: *C1C[C@H]2CC[C@@H](C1)N2*]

In some embodiments, -Cy- is

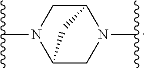 [SMILES: *N1C[C@@H]2C[C@H]1CN2*]

In some embodiments, -Cy- is

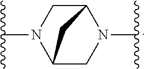 [SMILES: *N1C[C@H]2C[C@@H]1CN2*]

In some embodiments, -Cy- is

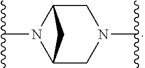 [SMILES: *N1C[C@H]2C[C@@H](C1)N2*]

In some embodiments, an optionally substituted group on -Cy- is selected from —F, —C₁₋₆alkyl, —OH, —OC₁₋₆alkyl, —CO₂H, —CO₂C₁₋₆alkyl, —(CH₂)₁₋₆CO₂H, —(CH₂)₁₋₆CO₂C₁₋₆alkyl, —P(O)(OH)₂, —P(O)(OC₁₋₆alkyl)₂, —(CH₂)₁₋₆P(O)(OH)₂, and —(CH₂)₁₋₆P(O)(OC₁₋₆alkyl)₂.

In some embodiments, -Cy- is selected from those depicted in Table 1, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1, below.

In some embodiments, L is substituted by a group selected from —F, —C₁₋₆alkyl, —OH, —OC₁₋₆alkyl, —CO₂H, —CO₂C₁₋₆alkyl, —(CH₂)₁₋₆CO₂H, —(CH₂)₁₋₆CO₂C₁₋₆alkyl, —P(O)(OH)₂, —P(O)(OC₁₋₆alkyl)₂, —(CH₂)₁₋₆P(O)(OH)₂, and —(CH₂)₁₋₆P(O)(OC₁₋₆alkyl)₂.

In some embodiments, L is —NR—(C₁₋₁₀ aliphatic)-. In some embodiments, L is —(C₁₋₁₀ aliphatic)-NR—(C₁₋₁₀ aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy- (C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀ aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is —Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀-Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is —Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is

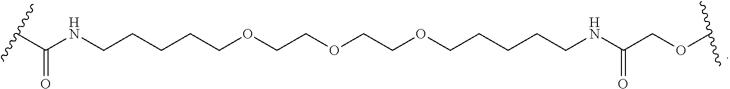 [SMILES: *OCC(=O)NCCCCCOCCOCCOCCCCCNC(*)=O]

In some embodiments, L is

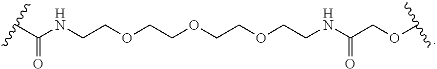 [SMILES: *OCC(=O)NCCOCCOCCOCCNC(*)=O]

In some embodiments, L is

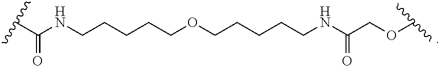 [SMILES: *OCC(=O)NCCCCCOCCCCCNC(*)=O]

In some embodiments, L is

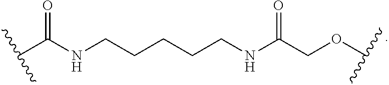 [SMILES: *OCC(=O)NCCCCCNC(*)=O]

In some embodiments, L is

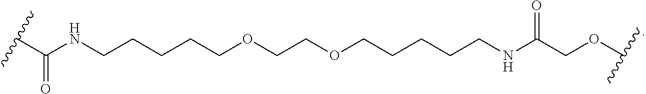 [SMILES: *OCC(=O)NCCCCCOCCOCCCCCNC(*)=O]

In some embodiments, L is

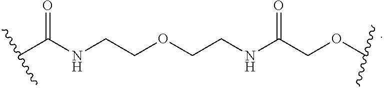 [SMILES: *OCC(=O)NCCOCCNC(*)=O]

In some embodiments, L is

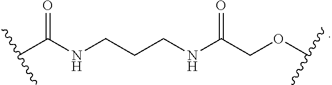 [SMILES: *OCC(=O)NCCCNC(*)=O]

In some embodiments, L is

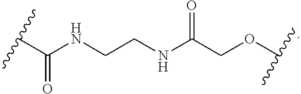 [SMILES: *OCC(=O)NCCNC(*)=O]

In some embodiments, L is

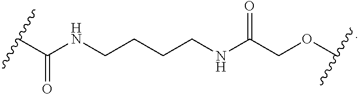 [SMILES: *OCC(=O)NCCCCNC(*)=O]

In some embodiments, L is

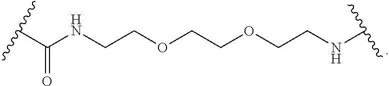 [SMILES: *NCCOCCOCCNC(*)=O]

In some embodiments, L is

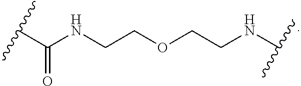 [SMILES: *NCCOCCNC(*)=O]

In some embodiments, L is

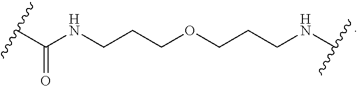 [SMILES: *NCCCOCCCNC(*)=O]

In some embodiments, L is

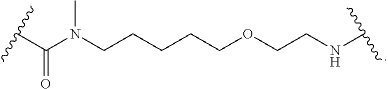 [SMILES: *NCCOCCCCCN(C)C(*)=O]

In some embodiments, L is

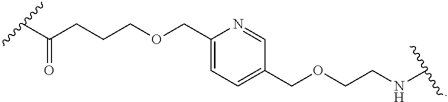 [SMILES: *NCCOCc1ccc(COCCCC(*)=O)nc1]

In some embodiments, L is

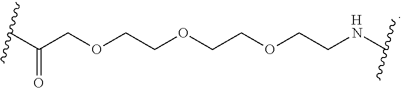

In some embodiments, L is

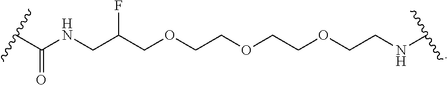

In some embodiments, L is

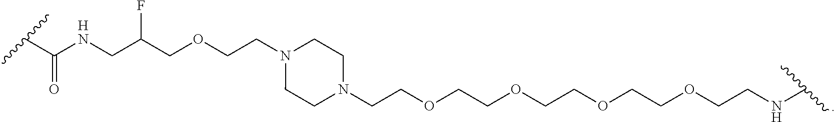

In some embodiments, L is

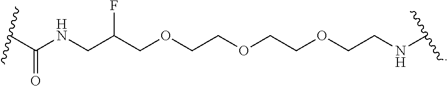

In some embodiments, L is

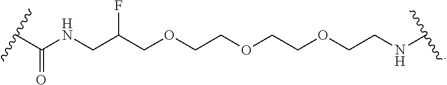

In some embodiments, L is

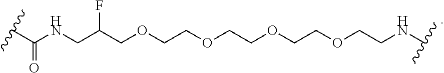

In some embodiments, L is

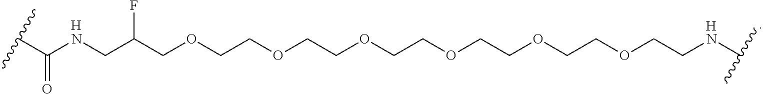

In some embodiments, L is

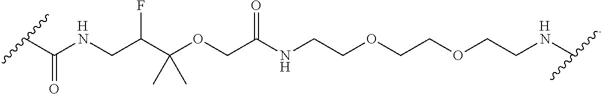

In some embodiments, L is

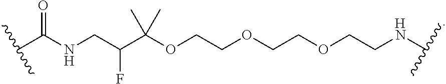

In some embodiments, L is

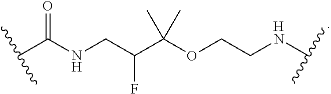

In some embodiments, L is

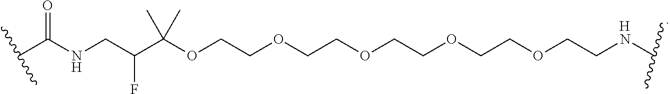

In some embodiments, L is

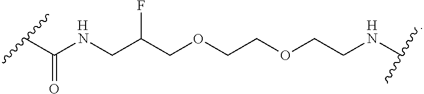

In some embodiments, L is

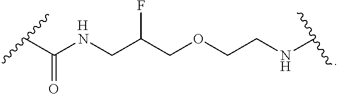

In some embodiments L is

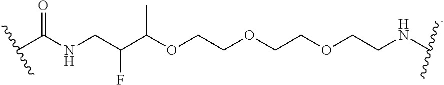 [SMILES: *NCCOCCOCCOC(C)C(F)CNC(*)=O]

In some embodiments, L is

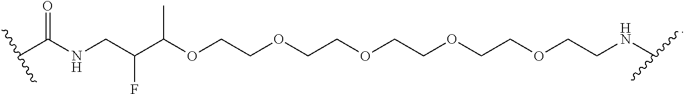 [SMILES: *NCCOCCOCCOCCOCCOC(C)C(F)CNC(*)=O]

In some embodiments, L is

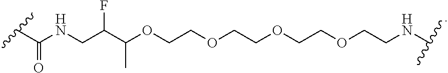 [SMILES: *NCCOCCOCCOCCOC(C)C(F)CNC(*)=O]

In some embodiments, L is

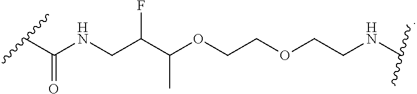 [SMILES: *NCCOCCOC(C)C(F)CNC(*)=O]

In some embodiments, L is

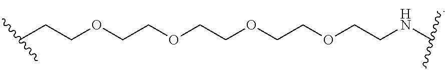 [SMILES: *CCOCCOCCOCCOCCN*]

In some embodiments, L is

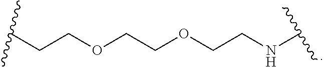 [SMILES: *CCOCCOCCN*]

In some embodiments, L is

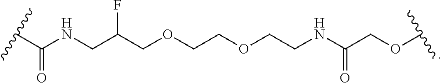 [SMILES: *OCC(=O)NCCOCCOCC(F)CNC(*)=O]

In some embodiments, L is

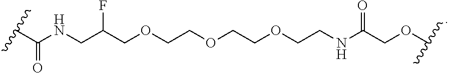 [SMILES: *OCC(=O)NCCOCCOCCOCC(F)CNC(*)=O]

In some embodiments, L is

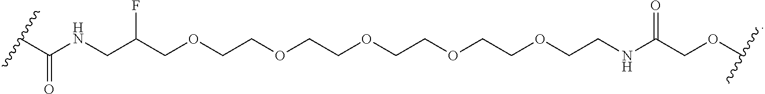 [SMILES: *OCC(=O)NCCOCCOCCOCCOCCOCC(F)CNC(*)=O]

In some embodiments, L is

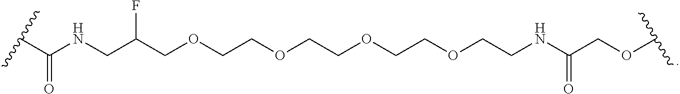 [SMILES: *OCC(=O)NCCOCCOCCOCCOCC(F)CNC(*)=O]

In some embodiments, L is

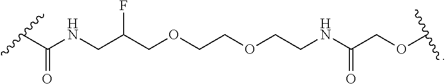 [SMILES: *OCC(=O)NCCOCCOCC(F)CNC(*)=O]

In some embodiments, L is

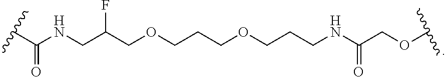 [SMILES: *OCC(=O)NCCCOCCCOCC(F)CNC(*)=O]

In some embodiments, L is

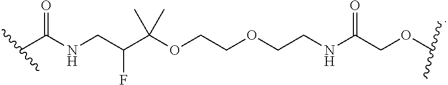 [SMILES: *OCC(=O)NCCOCCOC(C)(C)C(F)CNC(*)=O]

In some embodiments, L is

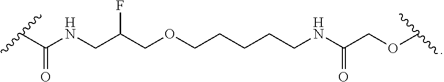 [SMILES: *OCC(=O)NCCCCCOCC(F)CNC(*)=O]

In some embodiments, L is

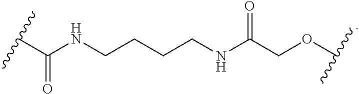 [SMILES: *OCC(=O)NCCCCNC(*)=O]

In some embodiments, L is

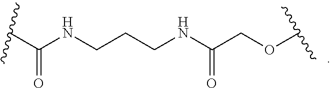 [SMILES: *OCC(=O)NCCCNC(*)=O]

In some embodiments, L is

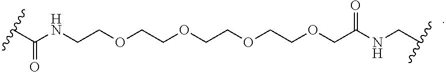 [SMILES: *CNC(=O)COCCOCCOCCOCCNC(*)=O]

In some embodiments, L is

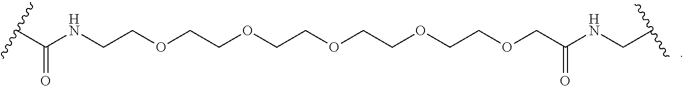 [SMILES: *CNC(=O)COCCOCCOCCOCCOCCNC(*)=O]

In some embodiments, L is

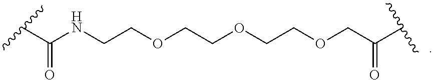 [SMILES: *C(=O)COCCOCCOCCNC(*)=O]

In some embodiments, L is

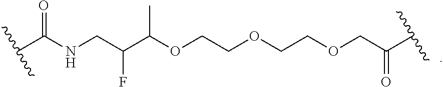 [SMILES: *C(=O)COCCOCCOC(C)C(F)CNC(*)=O]

In some embodiments, L is

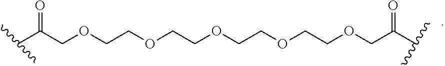 [SMILES: *C(=O)COCCOCCOCCOCCOCC(*)=O]

In some embodiments, L is

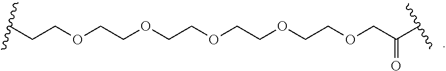 [SMILES: *CCOCCOCCOCCOCCOCC(*)=O]

In some embodiments, L is

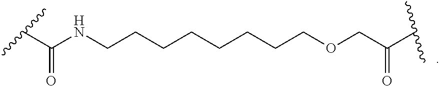 [SMILES: *C(=O)COCCCCCCCCNC(*)=O]

In some embodiments, L is

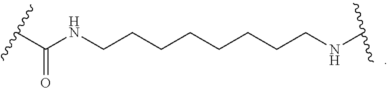 [SMILES: *NCCCCCCCCNC(*)=O]

In some embodiments, L is

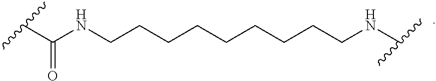 [SMILES: *NCCCCCCCCCNC(*)=O]

In some embodiments, L is

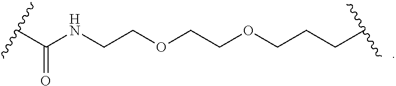 [SMILES: *CCCOCCOCCNC(*)=O]

In some embodiments, L is

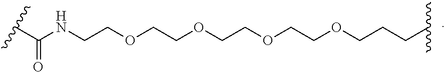 [SMILES: *CCCOCCOCCOCCOCCNC(*)=O]

In some embodiments, L is

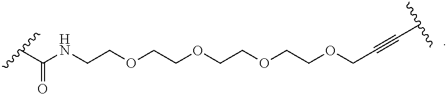 [SMILES: *C#CCOCCOCCOCCOCCNC(*)=O]

In some embodiments, L is

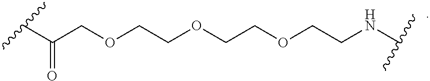 [SMILES: *NCCOCCOCCOCC(*)=O]

In some embodiments, L is

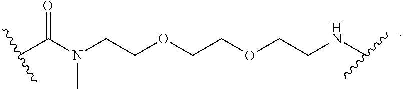 [SMILES: *NCCOCCOCCN(C)C(*)=O]

In some embodiments, L is

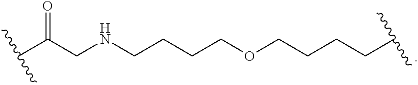 [SMILES: *CCCCOCCCCNCC(*)=O]

In some embodiments, L is

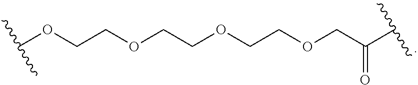 [SMILES: *OCCOCCOCCOCC(*)=O]

In some embodiments, L is

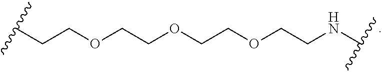 [SMILES: *CCOCCOCCOCCN*]

In some embodiments, L is

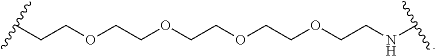 [SMILES: *CCOCCOCCOCCOCCN*]

In some embodiments, L is

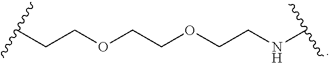 [SMILES: *CCOCCOCCN*]

In some embodiments, L is

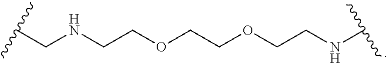 [SMILES: *CNCCOCCOCCN*]

In some embodiments, L is

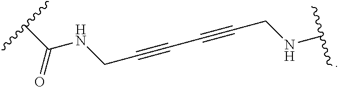 [SMILES: *NCC#CC#CCNC(*)=O]

In some embodiments, L is

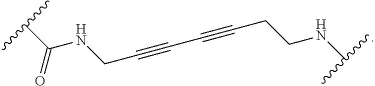 [SMILES: *NCCC#CC#CCNC(*)=O]

In some embodiments, L is

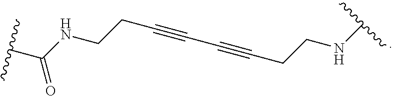 [SMILES: *NCCC#CC#CCCNC(*)=O]

In some embodiments, L is

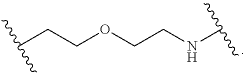 [SMILES: *CCOCCN*]

In some embodiments, L is

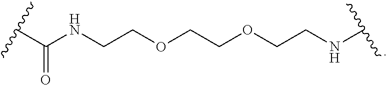 [SMILES: *NCCOCCOCCNC(*)=O]

In some embodiments, L is

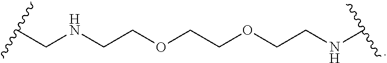 [SMILES: *CNCCOCCOCCN*]

In some embodiments, L is

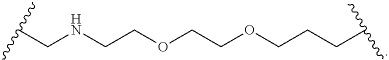 [SMILES: *CCCOCCOCCNC*]

In some embodiments, L is

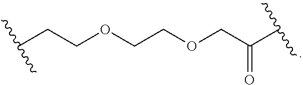 [SMILES: *CCOCCOCC(*)=O]

In some embodiments, L is

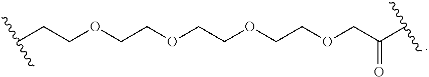 [SMILES: *CCOCCOCCOCCOCC(*)=O]

In some embodiments, L is

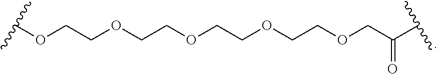 [SMILES: *OCCOCCOCCOCCOCC(*)=O]

In some embodiments, L is

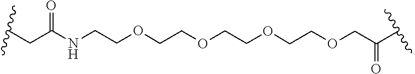 [SMILES: *CC(=O)NCCOCCOCCOCCOCC(*)=O]

In some embodiments, L is

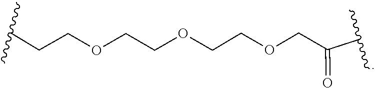 [SMILES: *CCOCCOCCOCC(*)=O]

In some embodiments, L is

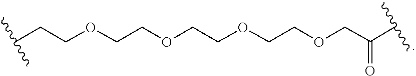 [SMILES: *CCOCCOCCOCCOCC(*)=O]

In some embodiments, L is

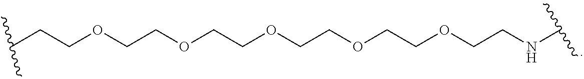 [SMILES: *CCOCCOCCOCCOCCOCCN*]

In some embodiments, L is

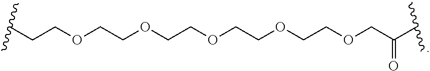 [SMILES: *CCOCCOCCOCCOCCOCC(*)=O]

In some embodiments, L is

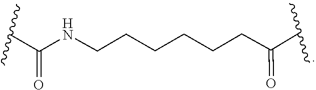 [SMILES: *C(=O)CCCCCCNC(*)=O]

In some embodiments, L is

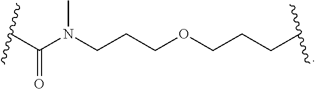 [SMILES: *CCCOCCCN(C)C(*)=O]

In some embodiments, L is

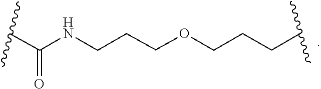 [SMILES: *CCCOCCCNC(*)=O]

In some embodiments, L is

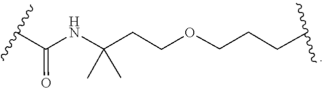 [SMILES: *CCCOCCC(C)(C)NC(*)=O]

In some embodiments, L is

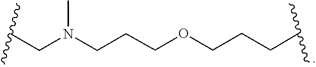 [SMILES: *CCCOCCCN(C)C*]

In some embodiments, L is

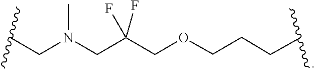 [SMILES: *CCCOCC(F)(F)CN(C)C*]

In some embodiments, L is

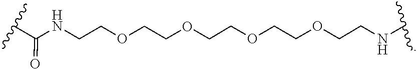 [SMILES: *NCCOCCOCCOCCOCCNC(*)=O]

In some embodiments, L is

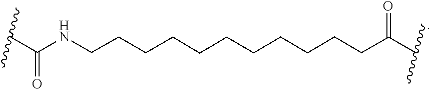 [SMILES: *C(=O)CCCCCCCCCCCNC(*)=O]

In some embodiments, L is

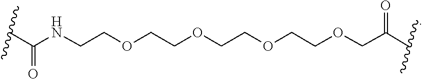 [SMILES: *C(=O)COCCOCCOCCOCCNC(*)=O]

In some embodiments, L is

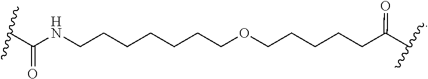 [SMILES: *C(=O)CCCCCOCCCCCCCNC(*)=O]

In some embodiments, L is

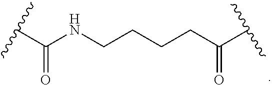 [SMILES: *C(=O)CCCCNC(*)=O]

In some embodiments, L is

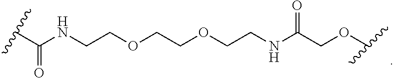 [SMILES: *OCC(=O)NCCOCCOCCNC(*)=O]

In some embodiments, L is

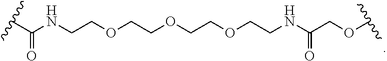 [SMILES: *OCC(=O)NCCOCCOCCOCCNC(*)=O]

In some embodiments, L is

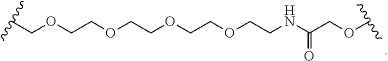 [SMILES: *COCCOCCOCCOCCNC(=O)CO*]

In some embodiments, L is

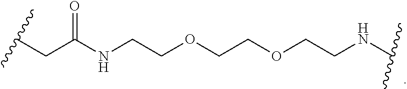 [SMILES: *CC(=O)NCCOCCOCCN*]

In some embodiments, L is

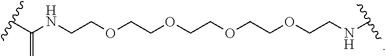 [SMILES: *NCCOCCOCCOCCOCCNC(*)=C]

In some embodiments, L is

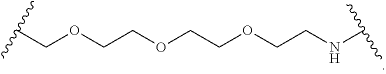 [SMILES: *COCCOCCOCCN*]

In some embodiments, L is

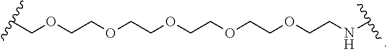 [SMILES: *COCCOCCOCCOCCOCCN*]

In some embodiments, L is

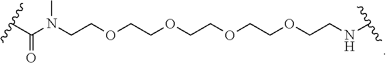 [SMILES: *NCCOCCOCCOCCOCCN(C)C(*)=O]

In some embodiments, L is

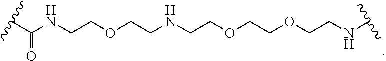 [SMILES: *NCCOCCOCCNCCOCCNC(*)=O]

In some embodiments, L is

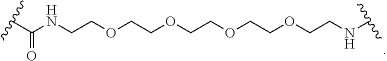 [SMILES: *NCCOCCOCCOCCOCCNC(*)=O]

In some embodiments, L is

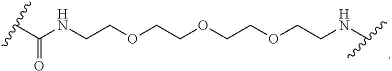 [SMILES: *NCCOCCOCCOCCNC(*)=O]

In some embodiments, L is

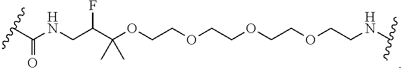 [SMILES: *NCCOCCOCCOCCOC(C)(C)C(F)CNC(*)=O]

In some embodiments, L is

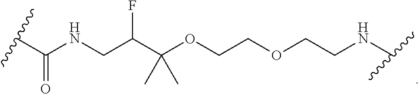 [SMILES: *NCCOCCOC(C)(C)C(F)CNC(*)=O]

In some embodiments, L is

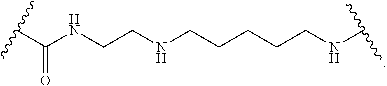 [SMILES: *NCCCCCNCCNC(*)=O]

In some embodiments, L is

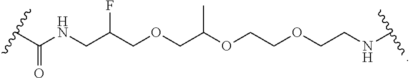 [SMILES: *NCCOCCOC(C)COCC(F)CNC(*)=O]

In some embodiments, L is

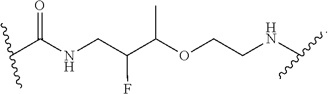 [SMILES: *NCCOC(C)C(F)CNC(*)=O]

In some embodiments, L is

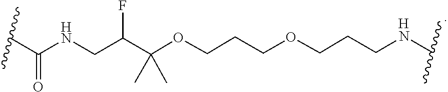

In some embodiments, L is

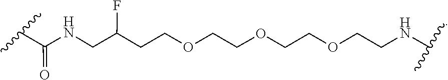

In some embodiments, L is

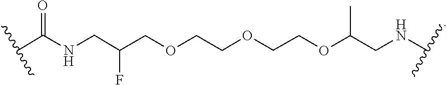 [SMILES: *NCC(C)OCCOCCOCC(F)CNC(*)=O]

In some embodiments, L is

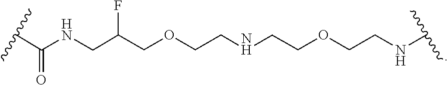 [SMILES: *NCCOCCNCCOCC(F)CNC(*)=O]

In some embodiments, L is

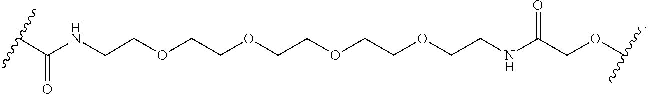 [SMILES: *OCC(=O)NCCOCCOCCOCCOCCNC(*)=O]

In some embodiments, L is

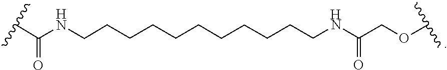

In some embodiments, L is

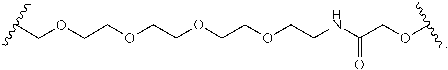

In some embodiments, L is

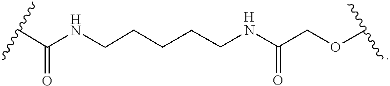

In some embodiments, L is

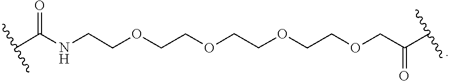 [SMILES: *C(=O)COCCOCCOCCOCCNC(*)=O]

In some embodiments, L is

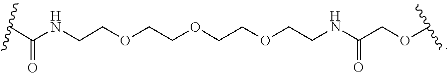

In some embodiments, L is

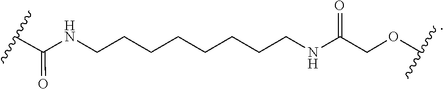 [SMILES: *OCC(=O)NCCCCCCCCNC(*)=O]

In some embodiments, L is

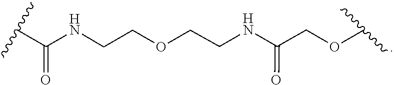 [SMILES: *OCC(=O)NCCOCCNC(*)=O]

In some embodiments, L is

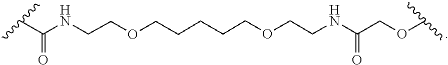 [SMILES: *OCC(=O)NCCOCCCCCOCCNC(*)=O]

In some embodiments, L is

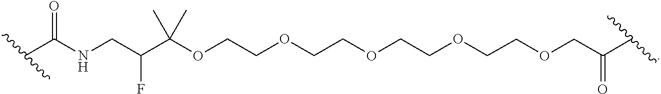 [SMILES: *C(=O)COCCOCCOCCOCCOC(C)(C)C(F)CNC(*)=O]

In some embodiments, L is

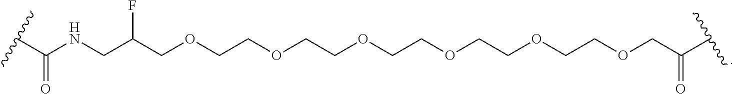 [SMILES: *C(=O)COCCOCCOCCOCCOCCOCC(F)CNC(*)=O]

In some embodiments, L is

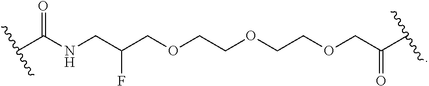 [SMILES: *C(=O)COCCOCCOCC(F)CNC(*)=O]

In some embodiments, L is

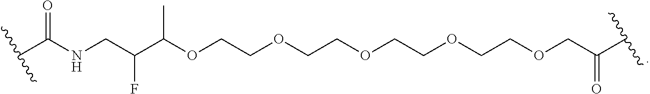 [SMILES: *C(=O)COCCOCCOCCOCCOC(C)C(F)CNC(*)=O]

In some embodiments, L is

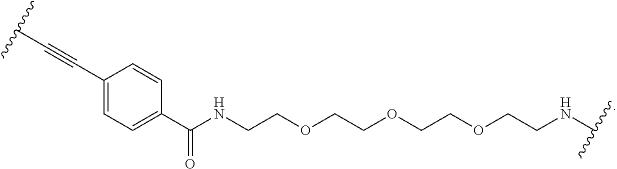 [SMILES: *C#Cc1ccc(C(=O)NCCOCCOCCOCCN*)cc1]

In some embodiments, L is

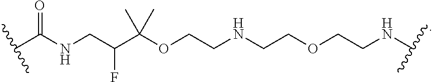 [SMILES: *NCCOCCNCCOC(C)(C)C(F)CNC(*)=O]

In some embodiments, L is

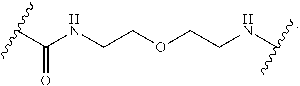 [SMILES: *NCCOCCNC(*)=O]

In some embodiments, L is

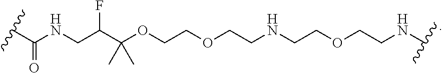 [SMILES: *NCCOCCNCCOCCOC(C)(C)C(F)CNC(*)=O]

In some embodiments, L is

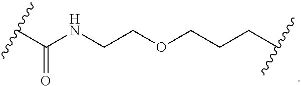 [SMILES: *CCCOCCNC(*)=O]

In some embodiments, L is

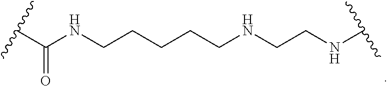 [SMILES: *NCCNCCCCCNC(*)=O]

In some embodiments, L is

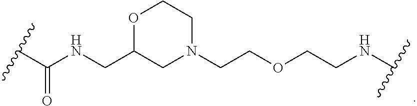 [SMILES: *NCCOCCN1CCOC(CNC(*)=O)C1]

In some embodiments, L is

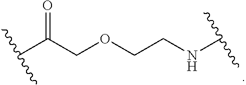 [SMILES: *NCCOCC(*)=O]

In some embodiments, L is

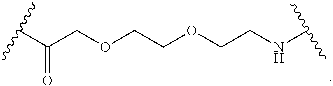 [SMILES: *NCCOCCOCC(*)=O]

In some embodiments, L is

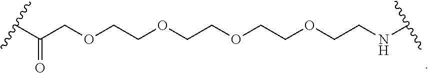 [SMILES: *NCCOCCOCCOCCOCC(*)=O]

In some embodiments, L is

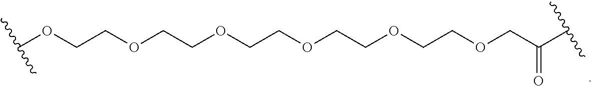 [SMILES: *OCCOCCOCCOCCOCCOCC(*)=O]

In some embodiments, L is

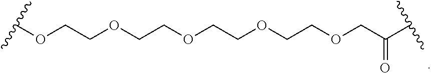 [SMILES: *OCCOCCOCCOCCOCC(*)=O]

In some embodiments, L is

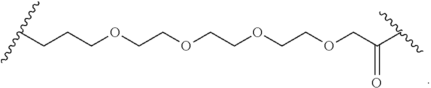 [SMILES: *CCCOCCOCCOCCOCC(*)=O]

In some embodiments, L is

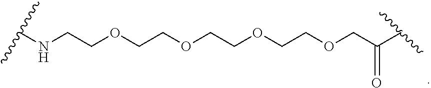 [SMILES: *NCCOCCOCCOCCOCC(*)=O]

In some embodiments, L is

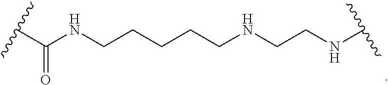 [SMILES: *NCCNCCCCCNC(*)=O]

In some embodiments L is

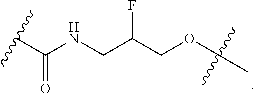 [SMILES: *OCC(F)CNC(*)=O]

In some embodiments, L is

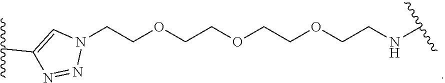 [SMILES: *NCCOCCOCCOCCn1cc(*)nn1]

In some embodiments, L is

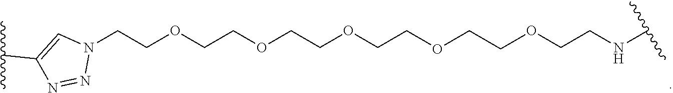 [SMILES: *NCCOCCOCCOCCOCCOCCn1cc(*)nn1]

In some embodiments, L is

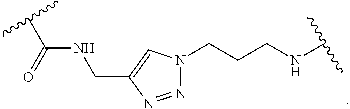 [SMILES: *NCCCn1cc(CNC(*)=O)nn1]

In some embodiments, L is

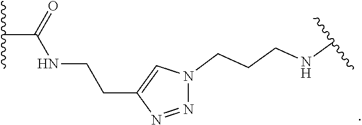 [SMILES: *NCCCn1cc(CCNC(*)=O)nn1]

In some embodiments, L is

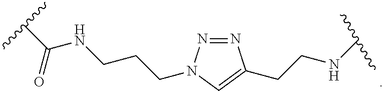 [SMILES: *NCCc1cn(CCCNC(*)=O)nn1]

In some embodiments, L is

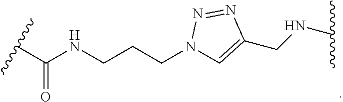 [SMILES: *NCc1cn(CCCNC(*)=O)nn1]

In some embodiments, L is

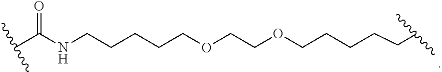 [SMILES: *CCCCCOCCOCCCCCNC(*)=O]

In some embodiments, L is

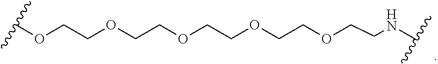 [SMILES: *NCCOCCOCCOCCOCCO*]

In some embodiments, L is

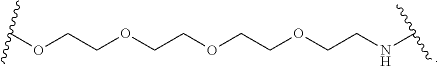 [SMILES: *NCCOCCOCCOCCO*]

In some embodiments, L is

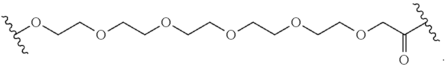 [SMILES: *OCCOCCOCCOCCOCCOCC(*)=O]

In some embodiments, L is

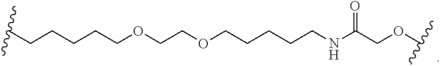 [SMILES: *CCCCCOCCOCCCCCNC(=O)CO*]

In some embodiments, L is

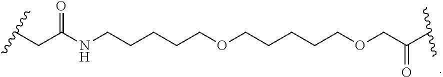 [SMILES: *CC(=O)NCCCCCOCCCCCOCC(*)=O]

In some embodiments, L is

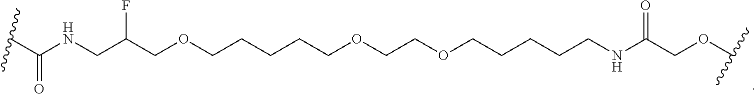 [SMILES: *OCC(=O)NCCCCCOCCOCCCCCOCC(F)CNC(*)=O]

In some embodiments, L is

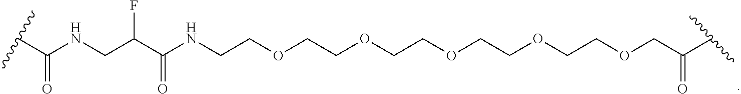 [SMILES: *C(=O)COCCOCCOCCOCCOCCNC(=O)C(F)CNC(*)=O]

In some embodiments, L is

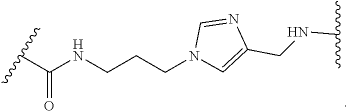 [SMILES: *NCc1cn(CCCNC(*)=O)cn1]

In some embodiments, L is

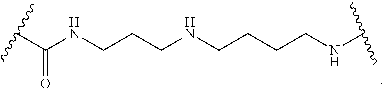 [SMILES: *NCCCCNCCCNC(*)=O]

In some embodiments, L is

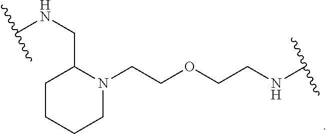 [SMILES: *NCCOCCN1CCCCC1CN*]

In some embodiments, L is

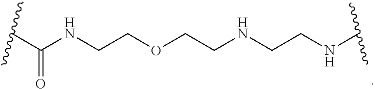 [SMILES: *NCCNCCOCCNC(*)=O]

In some embodiments, L is

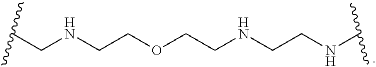 [SMILES: *CNCCOCCNCCN*]

In some embodiment, L is

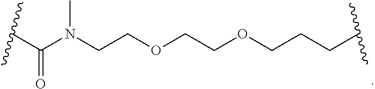 [SMILES: *CCCOCCOCCN(C)C(*)=O]

In some embodiment, L is

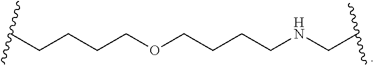 [SMILES: *CCCCOCCCCNC*]

In some embodiment, L is

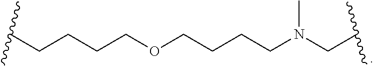 [SMILES: *CCCCOCCCCN(C)C*]

In some embodiments, L is

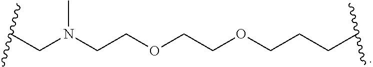 [SMILES: *CCCOCCOCCN(C)C*]

In some embodiments, L is

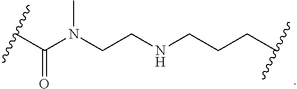 [SMILES: *CCCNCCN(C)C(*)=O]

In some embodiments, L is

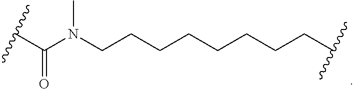 [SMILES: *CCCCCCCCN(C)C(*)=O]

In some embodiments, L is

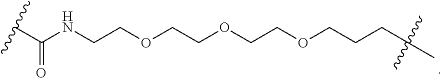 [SMILES: *CCCOCCOCCOCCNC(*)=O]

In some embodiments, L is

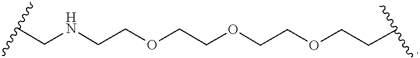 [SMILES: *CCOCCOCCOCCNC*]

In some embodiments, L is

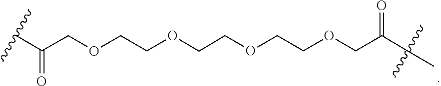 [SMILES: *C(=O)COCCOCCOCCOCC(*)=O]

In some embodiments, L is

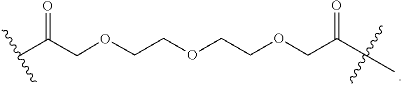 [SMILES: *C(=O)COCCOCCOCC(*)=O]

In some embodiments, L is

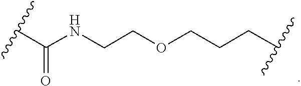 [SMILES: *CCCOCCNC(*)=O]

In some embodiments, L is

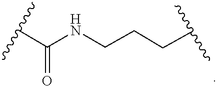 [SMILES: *CCCNC(*)=O]

In some embodiments, L is

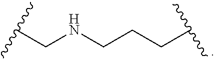 [SMILES: *CCCNC*]

In some embodiments, L is

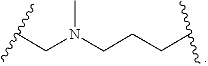 [SMILES: *CCCN(C)C*]

In some embodiments, L is

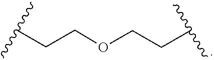 [SMILES: *CCOCC*]

In some embodiments, L is

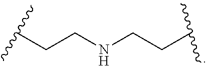 [SMILES: *CCNCC*]

In some embodiments, L is

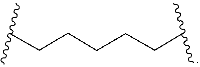 [SMILES: *CCCCC*]

In some embodiments, L is

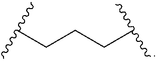 [SMILES: *CCC*]

In some embodiments, L is

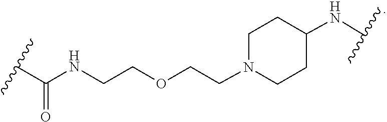 [SMILES: *NC1CCN(CCOCCNC(*)=O)CC1]

In some embodiments, L is

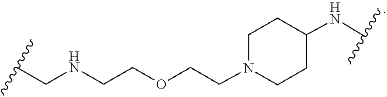 [SMILES: *CNCCOCCN1CCC(N*)CC1]

In some embodiments, L is

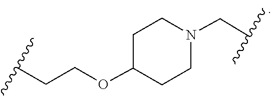 [SMILES: *CCOC1CCN(C*)CC1]

In some embodiments, L is

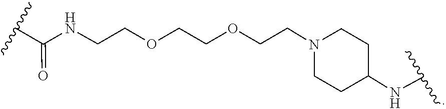 [SMILES: *NC1CCN(CCOCCOCCNC(*)=O)CC1]

In some embodiments, L is

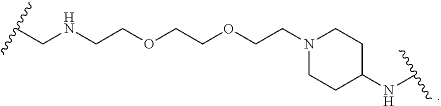 [SMILES: *CNCCOCCOCCN1CCC(N*)CC1]

In some embodiments, L is

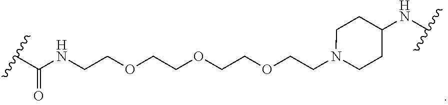 [SMILES: *NC1CCN(CCOCCOCCOCCNC(*)=O)CC1]

In some embodiments, L is

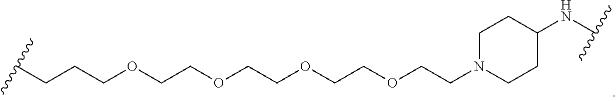 [SMILES: *CCCOCCOCCOCCOCCN1CCC(N*)CC1]

In some embodiments, L is

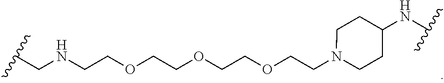 [SMILES: *CNCCOCCOCCOCCN1CCC(N*)CC1]

In some embodiments, L is

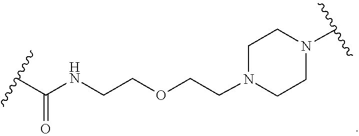 [SMILES: *C(=O)NCCOCCN1CCN(*)CC1]

In some embodiments, L is

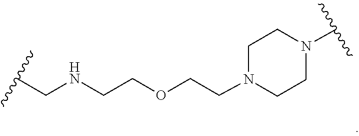 [SMILES: *CNCCOCCN1CCN(*)CC1]

In some embodiments, L is

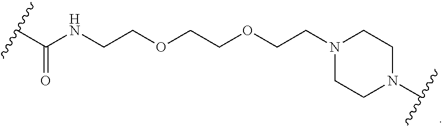

In some embodiments, L is

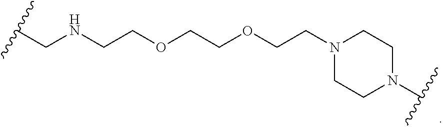

In some embodiments, L is

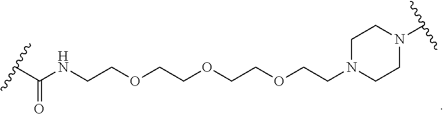 [SMILES: *C(=O)NCCOCCOCCOCCN1CCN(*)CC1]

In some embodiments, L is

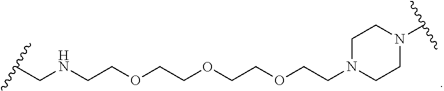 [SMILES: *CNCCOCCOCCOCCN1CCN(*)CC1]

In some embodiments, L is

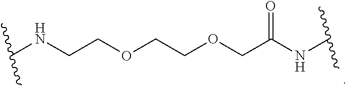 [SMILES: *NCCOCCOCC(=O)N*]

In some embodiments, L is

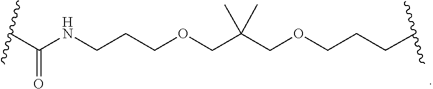 [SMILES: *CCCOCC(C)(C)COCCCNC(*)=O]

In some embodiments, L is

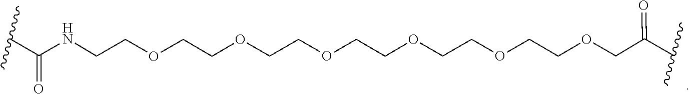 [SMILES: *C(=O)COCCOCCOCCOCCOCCOCCNC(*)=O]

In some embodiments, L is

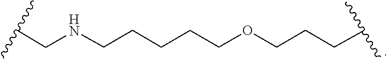 [SMILES: *CCCOCCCCCNC*]

In some embodiments, L is

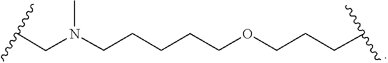 [SMILES: *CCCOCCCCCN(C)C*]

In some embodiments, L is

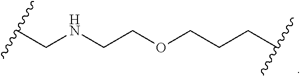 [SMILES: *CCCOCCNC*]

In some embodiments, L is

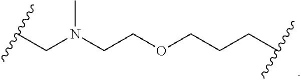 [SMILES: *CCCOCCN(C)C*]

In some embodiments, L is

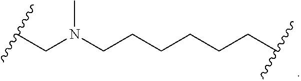 [SMILES: *CCCCCCN(C)C*]

In some embodiments, L is

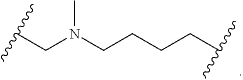 [SMILES: *CCCCN(C)C*]

In some embodiments, L is

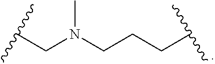 [SMILES: *CCCN(C)C*]

In some embodiments, L is

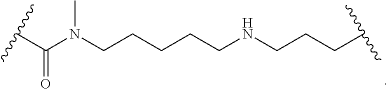 [SMILES: *CCCNCCCCCN(C)C(*)=O]

In some embodiments, L is

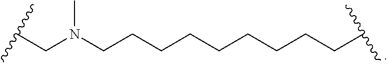 [SMILES: *CCCCCCCCCN(C)C*]

In some embodiments, L is

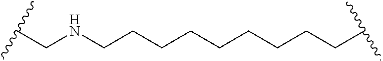

### In

some embodiments, L is

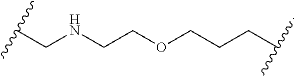

In some embodiments, L is

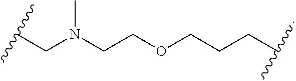

In some embodiments, L is

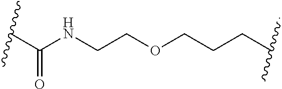

In some embodiments, L is

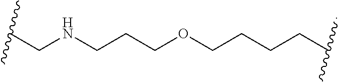

In some embodiments, L is

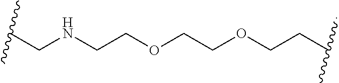

In some embodiments, L is

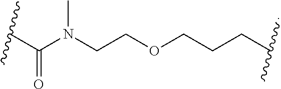

In some embodiments, L is

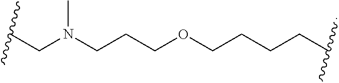

In some embodiments, L is

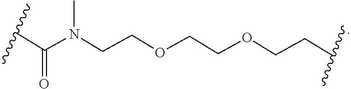 [SMILES: *CCOCCOCCN(C)C(*)=O]

In some embodiments, L is

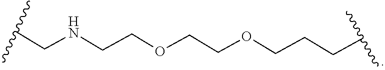 [SMILES: *CCCOCCOCCNC*]

### In

some embodiments, L is

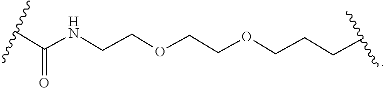 [SMILES: *CCCOCCOCCNC(*)=O]

In some embodiments, L is

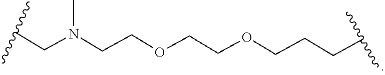 [SMILES: *CCCOCCOCCN(C)C*]

In some embodiments, L is

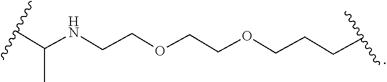 [SMILES: *CCCOCCOCCNC(*)C]

In some embodiments, L is

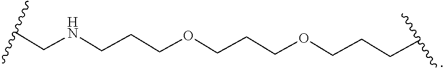 [SMILES: *CCCOCCCOCCCNC*]

In some embodiment, L is

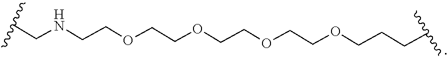 [SMILES: *CCCOCCOCCOCCOCCNC*]

In some embodiment, L is

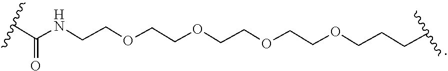 [SMILES: *CCCOCCOCCOCCOCCNC(*)=O]

In some embodiments, L is

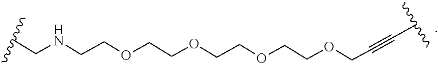 [SMILES: *C#CCOCCOCCOCCOCCNC*]

In some embodiments, L is

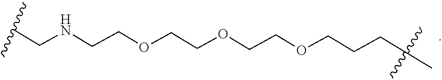 [SMILES: *CCCOCCOCCOCCNC*]

In some embodiments, L is

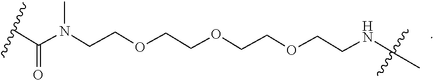 [SMILES: *NCCOCCOCCOCCN(C)C(*)=O]

In some embodiments, L is

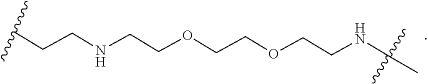 [SMILES: *CCNCCOCCOCCN*]

In some embodiments, L is

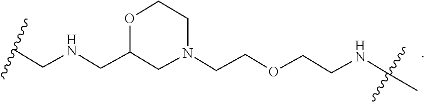 [SMILES: *CNCC1CN(CCOCCN*)CCO1]

In some embodiments, L is

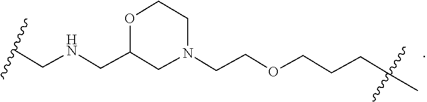 [SMILES: *CCCOCCN1CCOC(CNC*)C1]

In some embodiments, L is

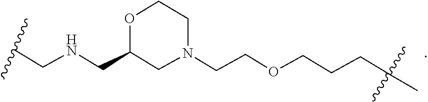 [SMILES: *CCCOCCN1CCO[C@H](CNC*)C1]

In some embodiments, L is

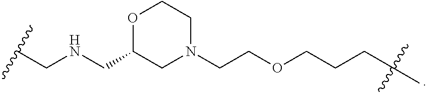 [SMILES: *CCCOCCN1CCO[C@@H](CNC*)C1]

In some embodiments, L is

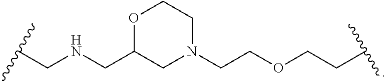 [SMILES: *CCOCCN1CCOC(CNC*)C1]

In some embodiments, L is

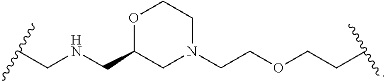 [SMILES: *CCOCCN1CCO[C@H](CNC*)C1]

In some embodiments, L is

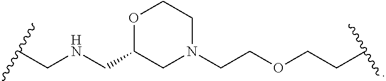 [SMILES: *CCOCCN1CCO[C@@H](CNC*)C1]

In some embodiments, L is

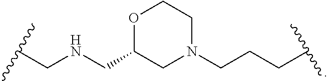 [SMILES: *CCCN1CCO[C@@H](CNC*)C1]

In some embodiments, L is

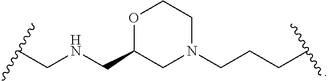 [SMILES: *CCCN1CCO[C@H](CNC*)C1]

In some embodiments, L is

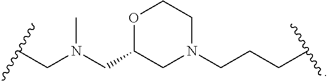 [SMILES: *CCCN1CCO[C@@H](CN(C)C*)C1]

In some embodiments, L is

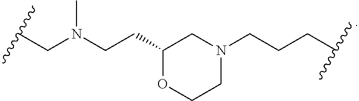 [SMILES: *CCCN1CCO[C@H](CCN(C)C*)C1]

In some embodiments, L is

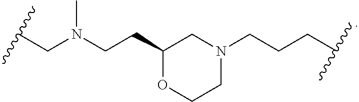 [SMILES: *CCCN1CCO[C@@H](CCN(C)C*)C1]

In some embodiments, L is

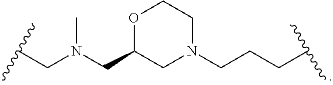 [SMILES: *CCCN1CCO[C@H](CN(C)C*)C1]

In some embodiments, L is

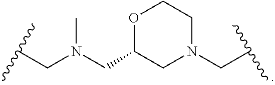 [SMILES: *CN(C)C[C@H]1CN(C*)CCO1]

In some embodiments, L is

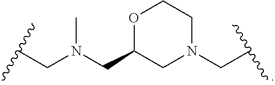 [SMILES: *CN(C)C[C@@H]1CN(C*)CCO1]

In some embodiments, L is

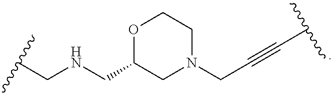 [SMILES: *C#CCN1CCO[C@@H](CNC*)C1]

In some embodiments, L is

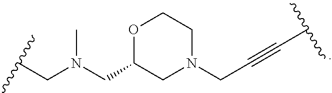 [SMILES: *C#CCN1CCO[C@@H](CN(C)C*)C1]

In some embodiments, L is

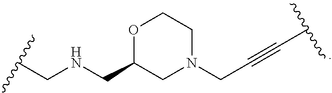 [SMILES: *C#CCN1CCO[C@H](CNC*)C1]

In some embodiments, L is

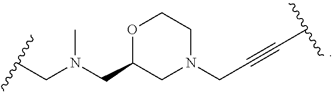 [SMILES: *C#CCN1CCO[C@H](CN(C)C*)C1]

In some embodiments, L is

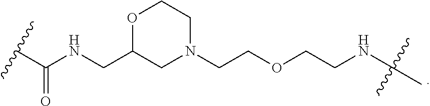 [SMILES: *NCCOCCN1CCOC(CNC(*)=O)C1]

In some embodiments, L is

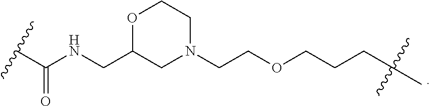 [SMILES: *CCCOCCN1CCOC(CNC(*)=O)C1]

In some embodiments, L is

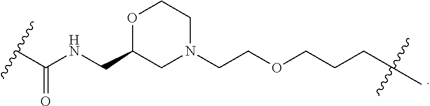 [SMILES: *CCCOCCN1CCO[C@H](CNC(*)=O)C1]

In some embodiments, L is

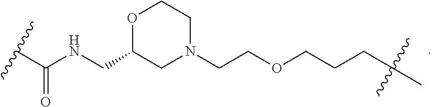 [SMILES: *CCCOCCN1CCO[C@@H](CNC(*)=O)C1]

In some embodiments, L is

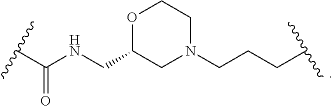 [SMILES: *CCCN1CCO[C@@H](CNC(*)=O)C1]

In some embodiments, L is

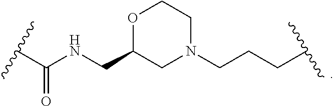 [SMILES: *CCCN1CCO[C@H](CNC(*)=O)C1]

In some embodiments, L is

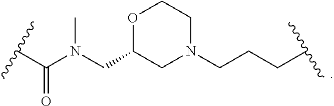 [SMILES: *CCCN1CCO[C@@H](CN(C)C(*)=O)C1]

In some embodiments, L is

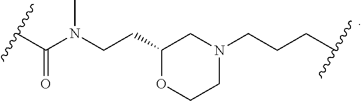 [SMILES: *CCCN1CCO[C@H](CCN(C)C(*)=O)C1]

In some embodiments, L is

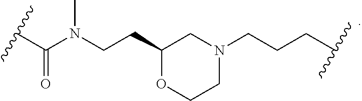 [SMILES: *CCCN1CCO[C@@H](CCN(C)C(*)=O)C1]

In some embodiments, L is

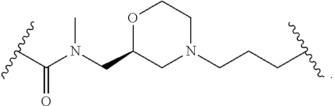 [SMILES: *CCCN1CCO[C@H](CN(C)C(*)=O)C1]

In some embodiments, L is

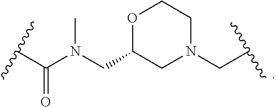 [SMILES: *CN1CCO[C@@H](CN(C)C(*)=O)C1]

In some embodiments, L is

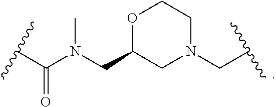 [SMILES: *CN1CCO[C@H](CN(C)C(*)=O)C1]

In some embodiments, L is

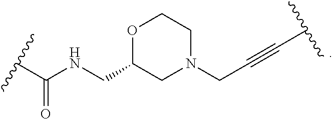 [SMILES: *C#CCN1CCO[C@@H](CNC(*)=O)C1]

In some embodiments, L is

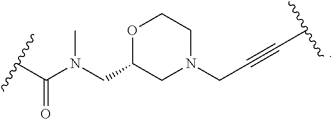 [SMILES: *C#CCN1CCO[C@@H](CN(C)C(*)=O)C1]

In some embodiments, L is

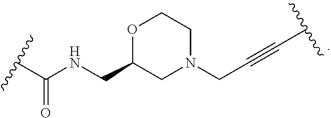 [SMILES: *C#CCN1CCO[C@H](CNC(*)=O)C1]

In some embodiments, L is

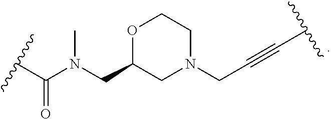 [SMILES: *C#CCN1CCO[C@H](CN(C)C(*)=O)C1]

In some embodiments, L is

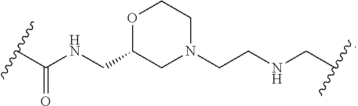 [SMILES: *CNCCN1CCO[C@@H](CNC(*)=O)C1]

In some embodiments, L is

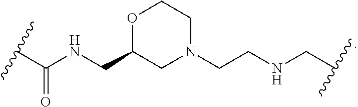 [SMILES: *CNCCN1CCO[C@H](CNC(*)=O)C1]

In some embodiments, L is

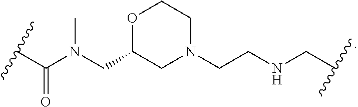 [SMILES: *CNCCN1CCO[C@@H](CN(C)C(*)=O)C1]

In some embodiments, L is

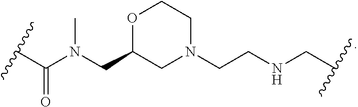 [SMILES: *CNCCN1CCO[C@H](CN(C)C(*)=O)C1]

In some embodiments, L is

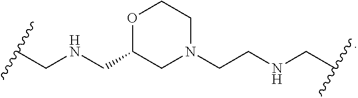 [SMILES: *CNCCN1CCO[C@@H](CNC*)C1]

In some embodiments, L is

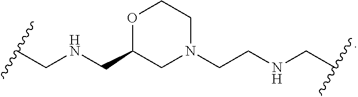 [SMILES: *CNCCN1CCO[C@H](CNC*)C1]

In some embodiments, L is

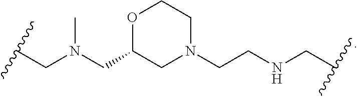 [SMILES: *CNCCN1CCO[C@@H](CN(C)C*)C1]

In some embodiments, L is

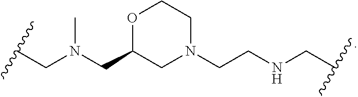 [SMILES: *CNCCN1CCO[C@H](CN(C)C*)C1]

In some embodiments, L is

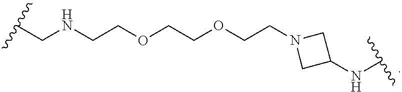 [SMILES: *CNCCOCCOCCN1CC(N*)C1]

In some embodiments, L is

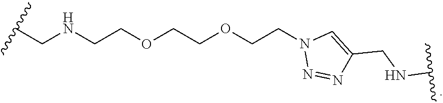 [SMILES: *CNCCOCCOCCn1cc(CN*)nn1]

In some embodiments, L is

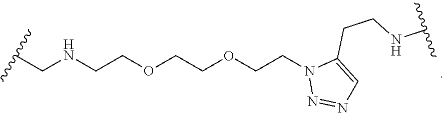 [SMILES: *CNCCOCCOCCn1nncc1CCN*]

In some embodiments, L is

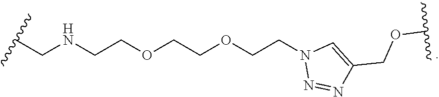 [SMILES: *CNCCOCCOCCn1cc(CO*)nn1]

In some embodiments, L is

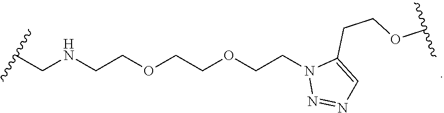 [SMILES: *CNCCOCCOCCn1nncc1CCO*]

In some embodiments, L is

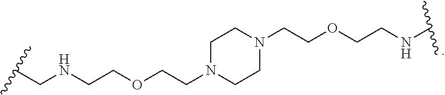 [SMILES: *CNCCOCCN1CCN(CCOCCN*)CC1]

In some embodiments, L is

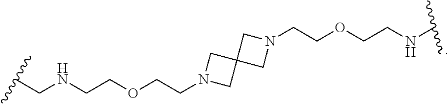 [SMILES: *CNCCOCCN1CC2(CN(CCOCCN*)C2)C1]

In some embodiments, L is

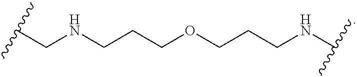 [SMILES: *CNCCCOCCCN*]

In some embodiments, L is

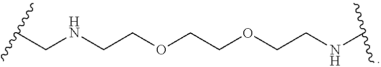 [SMILES: *CNCCOCCOCCN*]

In some embodiments, L is

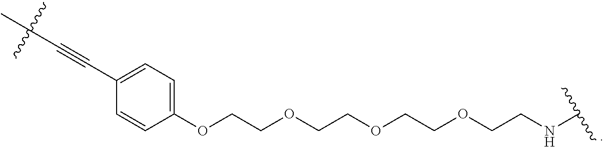 [SMILES: *C#Cc1ccc(OCCOCCOCCOCCN*)cc1]

In some embodiments, L is

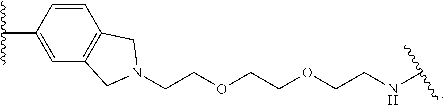 [SMILES: *NCCOCCOCCN1Cc2ccc(*)cc2C1]

In some embodiments, L is

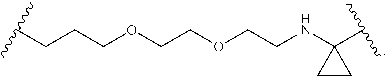 [SMILES: *CCCOCCOCCNC1(*)CC1]

In some embodiments, L is

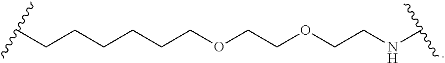 [SMILES: *CCCCCCOCCOCCN*]

In some embodiments, L is

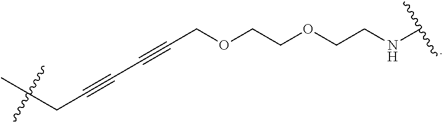 [SMILES: *CC#CC#CCOCCOCCN*]

In some embodiments, L is

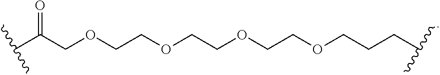 [SMILES: *CCCOCCOCCOCCOCC(*)=O]

In some embodiments, L is

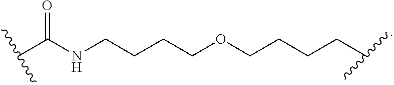 [SMILES: *CCCCOCCCCNC(*)=O]

In some embodiments, L is

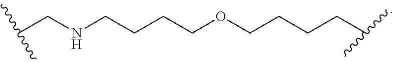 [SMILES: *CCCCOCCCCNC*]

In some embodiments, L is

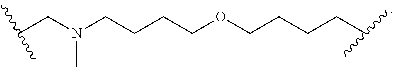 [SMILES: *CCCCOCCCCN(C)C*]

In some embodiments, L is

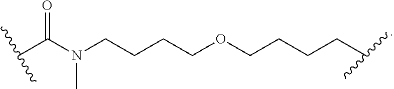 [SMILES: *CCCCOCCCCN(C)C(*)=O]

In some embodiments, L is

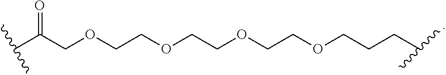 [SMILES: *CCCOCCOCCOCCOCC(*)=O]

In some embodiments, L is

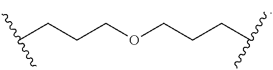 [SMILES: *CCCOCCC*]

In some embodiments, L is

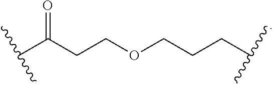 [SMILES: *CCCOCCC(*)=O]

In some embodiments, L is

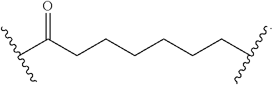 [SMILES: *CCCCCCC(*)=O]

In some embodiments, L is

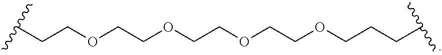 [SMILES: *CCCOCCOCCOCCOCC*]

In some embodiments, L is

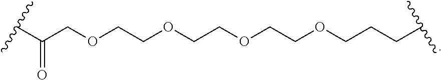 [SMILES: *CCCOCCOCCOCCOCC(*)=O]

In some embodiments, L is

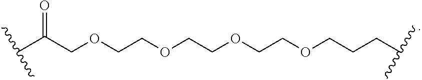 [SMILES: *CCCOCCOCCOCCOCC(*)=O]

In some embodiments, L is

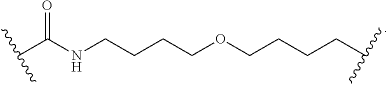 [SMILES: *CCCCOCCCCNC(*)=O]

In some embodiments, L is

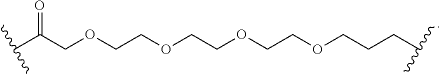 [SMILES: *CCCOCCOCCOCCOCC(*)=O]

In some embodiments, L is

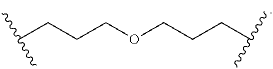 [SMILES: *CCCOCCC*]

In some embodiments, L is

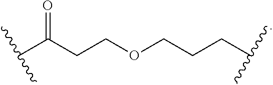 [SMILES: *CCCOCCC(*)=O]

In some embodiments, L is

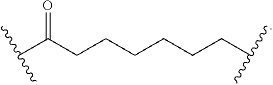 [SMILES: *CCCCCCC(*)=O]

In some embodiments, L is

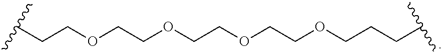 [SMILES: *CCCOCCOCCOCCOCC*]

In some embodiments, L is

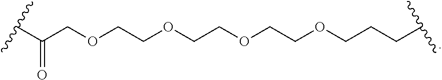 [SMILES: *CCCOCCOCCOCCOCC(*)=O]

In some embodiments, L is

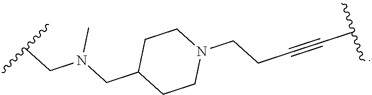 [SMILES: *C#CCCN1CCC(CN(C)C*)CC1]

In some embodiments, L is

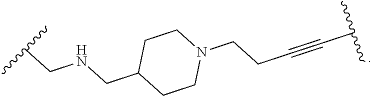 [SMILES: *C#CCCN1CCC(CNC*)CC1]

In some embodiments, L is

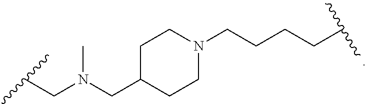 [SMILES: *CCCCN1CCC(CN(C)C*)CC1]

In some embodiments, L is

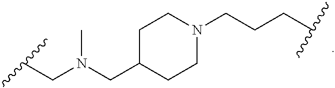 [SMILES: *CCCN1CCC(CN(C)C*)CC1]

In some embodiments, L is

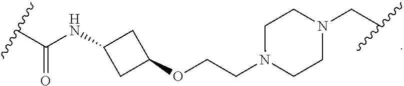 [SMILES: *CN1CCN(CCO[C@H]2C[C@H](NC(*)=O)C2)CC1]

### In

some embodiments, L is

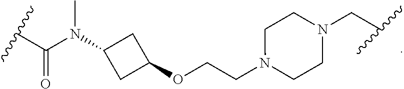 [SMILES: *CN1CCN(CCO[C@H]2C[C@H](N(C)C(*)=O)C2)CC1]

In some embodiments, L is

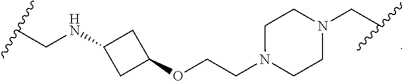 [SMILES: *CN[C@H]1C[C@H](OCCN2CCN(C*)CC2)C1]

In some embodiments, L is

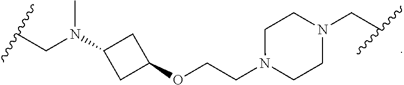 [SMILES: *CN1CCN(CCO[C@H]2C[C@H](N(C)C*)C2)CC1]

In some embodiments, L is

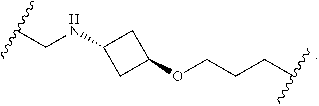 [SMILES: *CCCO[C@H]1C[C@H](NC*)C1]

In some embodiments, L is

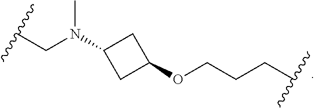 [SMILES: *CCCO[C@H]1C[C@H](N(C)C*)C1]

In some embodiments, L is

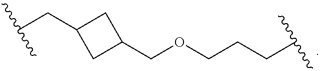 [SMILES: *CCCOCC1CC(C*)C1]

In some embodiments, L is

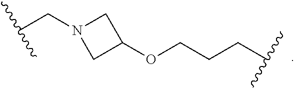 [SMILES: *CCCOC1CN(C*)C1]

In some embodiments, L is

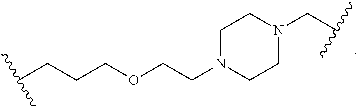 [SMILES: *CCCOCCN1CCN(C*)CC1]

In some embodiments, L is

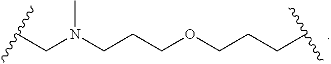 [SMILES: *CCCOCCCN(C)C*]

In some embodiments, L is

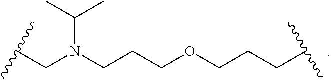 [SMILES: *CCCOCCCN(C*)C(C)C]

In some embodiments, L is

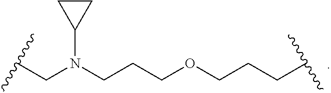 [SMILES: *CCCOCCCN(C*)C1CC1]

In some embodiments, L is

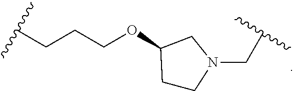 [SMILES: *CCCO[C@@H]1CCN(C*)C1]

In some embodiments, L is

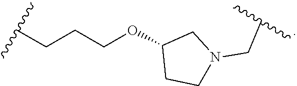 [SMILES: *CCCO[C@H]1CCN(C*)C1]

In some embodiments, L is

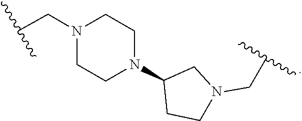 [SMILES: *CN1CCN([C@@H]2CCN(C*)C2)CC1]

In some embodiments, L is

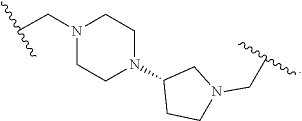 [SMILES: *CN1CCN([C@H]2CCN(C*)C2)CC1]

In some embodiments, L is

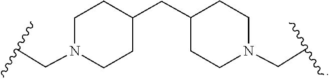 [SMILES: *CN1CCC(CC2CCN(C*)CC2)CC1]

In some embodiments, L is

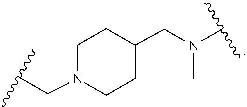

In some embodiments, L is

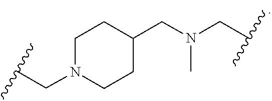 [SMILES: *CN(C)CC1CCN(C*)CC1]

In some embodiments, L is

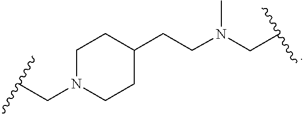 [SMILES: *CN(C)CCC1CCN(C*)CC1]

In some embodiments, L is

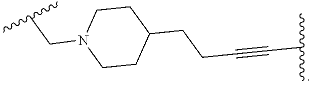 [SMILES: *C#CCCC1CCN(C*)CC1]

In some embodiments, L is

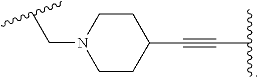 [SMILES: *C#CC1CCN(C*)CC1]

In some embodiments, L is

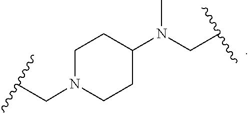 [SMILES: *CN1CCC(N(C)C*)CC1]

In some embodiments, L is

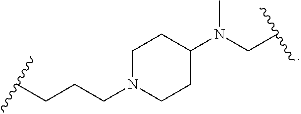 [SMILES: *CCCN1CCC(N(C)C*)CC1]

In some embodiments, L is

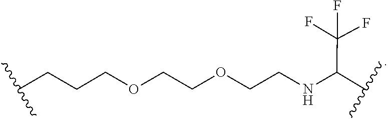 [SMILES: *CCCOCCOCCNC(*)C(F)(F)F]

In some embodiments, L is

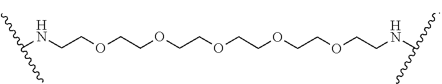 [SMILES: *NCCOCCOCCOCCOCCOCCN*]

In some embodiments, L is

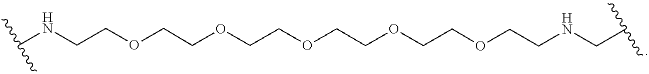 [SMILES: *CNCCOCCOCCOCCOCCOCCN*]

In some embodiments, L is

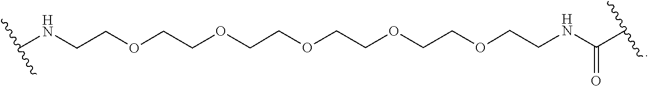 [SMILES: *NCCOCCOCCOCCOCCOCCNC(*)=O]

In some embodiments, L is

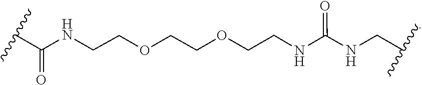 [SMILES: *CNC(=O)NCCOCCOCCNC(*)=O]

In some embodiments, L is

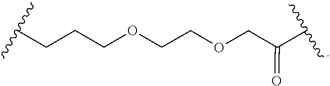 [SMILES: *CCCOCCOCC(*)=O]

In some embodiments, L is

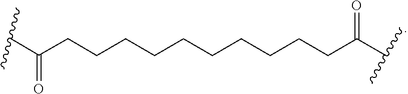 [SMILES: *C(=O)CCCCCCCCCCC(*)=O]

In some embodiments, L is

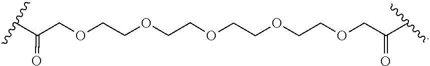 [SMILES: *C(=O)COCCOCCOCCOCCOCC(*)=O]

In some embodiments, L is

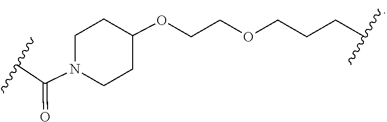 [SMILES: *CCCOCCOC1CCN(C(*)=O)CC1]

In some embodiments, L is

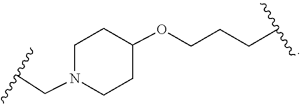 [SMILES: *CCCOC1CCN(C*)CC1]

In some embodiments, L is

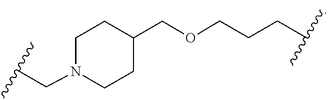 [SMILES: *CCCOCC1CCN(C*)CC1]

In some embodiments, L is

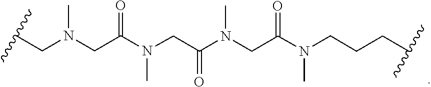 [SMILES: *CCCN(C)C(=O)CN(C)C(=O)CN(C)C(=O)CN(C)C*]

In some embodiments, L is

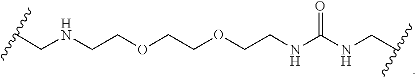 [SMILES: *CNCCOCCOCCNC(=O)NC*]

In some embodiment, L is

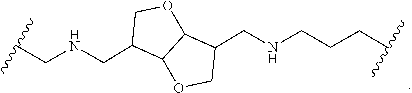 [SMILES: *CCCNCC1COC2C(CNC*)COC12]

In some embodiments, L is

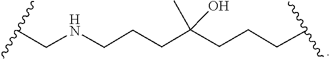 [SMILES: *CCCC(C)(O)CCCNC*]

In some embodiments, L is

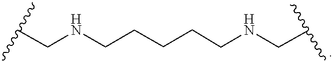 [SMILES: *CNCCCCCNC*]

In some embodiments, L is

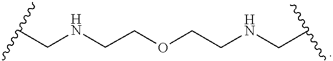 [SMILES: *CNCCOCCNC*]

In some embodiments, L is

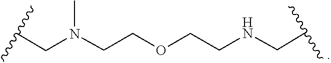 [SMILES: *CNCCOCCN(C)C*]

In some embodiments, L is

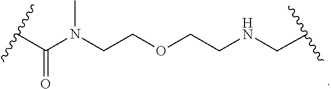 [SMILES: *CNCCOCCN(C)C(*)=O]

In some embodiments, L is

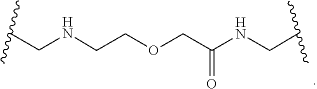 [SMILES: *CNCCOCC(=O)NC*]

In some embodiments, L is

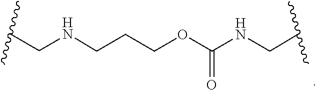 [SMILES: *CNCCCOC(=O)NC*]

In some embodiments, L is

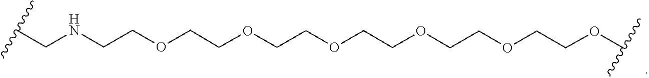 [SMILES: *CNCCOCCOCCOCCOCCOCCO*]

In some embodiments, L is

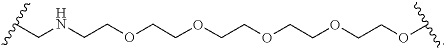 [SMILES: *CNCCOCCOCCOCCOCCO*]

In some embodiments, L is

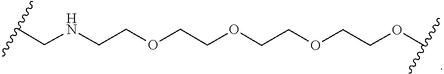 [SMILES: *CNCCOCCOCCOCCO*]

In some embodiments, L is

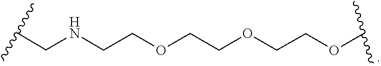 [SMILES: *CNCCOCCOCCO*]

In some embodiments, L is

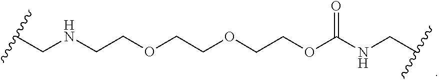 [SMILES: *CNCCOCCOCCOC(=O)NC*]

In some embodiments, L is

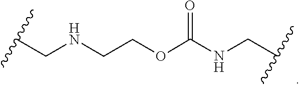 [SMILES: *CNCCOC(=O)NC*]

In some embodiments, L is

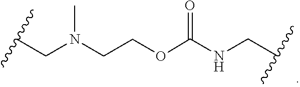 [SMILES: *CNC(=O)OCCN(C)C*]

In some embodiments, L is

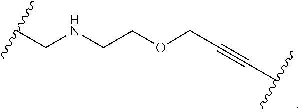 [SMILES: *C#CCOCCNC*]

In some embodiments, L is

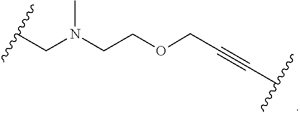 [SMILES: *C#CCOCCN(C)C*]

In some embodiments, L is

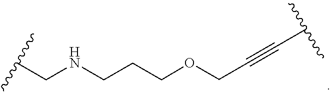 [SMILES: *C#CCOCCCNC*]

In some embodiments, L is

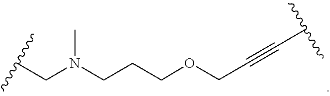 [SMILES: *C#CCOCCCN(C)C*]

In some embodiments, L is

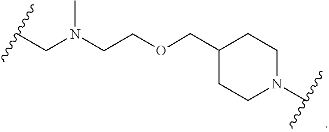 [SMILES: *CN(C)CCOCC1CCN(*)CC1]

In some embodiments, L is

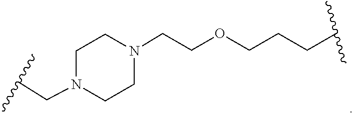 [SMILES: *CCCOCCN1CCN(C*)CC1]

In some embodiments, L is

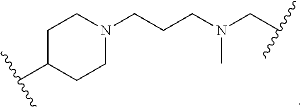 [SMILES: *CN(C)CCCN1CCC(*)CC1]

In some embodiments, L is

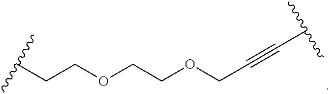 [SMILES: *C#CCOCCOCC*]

In some embodiments, L is

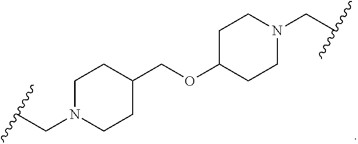 [SMILES: *CN1CCC(COC2CCN(C*)CC2)CC1]

In some embodiments, L is

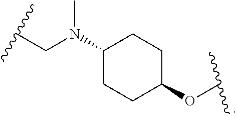 [SMILES: *CN(C)[C@H]1CC[C@H](O*)CC1]

In some embodiments, L is

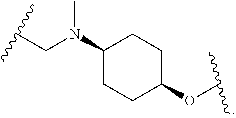 [SMILES: *CN(C)[C@H]1CC[C@@H](O*)CC1]

In some embodiments, L is

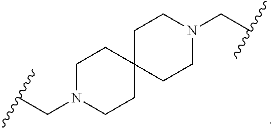 [SMILES: *CN1CCC2(CC1)CCN(C*)CC2]

In some embodiments, L is

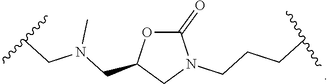 [SMILES: *CCCN1C[C@@H](CN(C)C*)OC1=O]

In some embodiments, L is

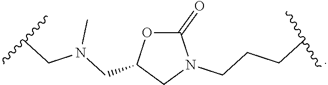 [SMILES: *CCCN1C[C@H](CN(C)C*)OC1=O]

In some embodiments, L is

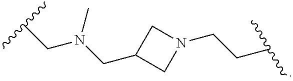 [SMILES: *CCN1CC(CN(C)C*)C1]

In some embodiments, L is

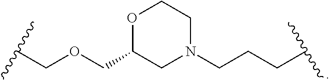 [SMILES: *CCCN1CCO[C@@H](COC*)C1]

In some embodiments, L is

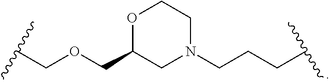 [SMILES: *CCCN1CCO[C@H](COC*)C1]

In some embodiments, L is

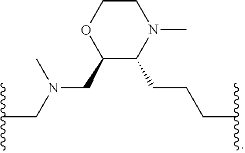 [SMILES: *CCC[C@@H]1[C@@H](CN(C)C*)OCCN1C]

In some embodiments, L is

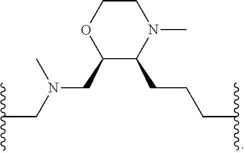 [SMILES: *CCC[C@H]1[C@@H](CN(C)C*)OCCN1C]

In some embodiments, L is

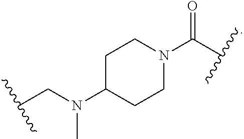 [SMILES: *CN(C)C1CCN(C(*)=O)CC1]

In some embodiments, L is

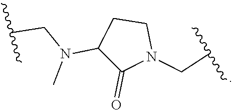 [SMILES: *CN1CCC(N(C)C*)C1=O]

In some embodiments, L is

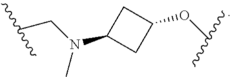 [SMILES: *CN(C)[C@H]1C[C@H](O*)C1]

In some embodiments, L is

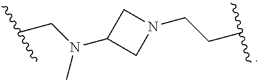 [SMILES: *CCN1CC(N(C)C*)C1]

In some embodiments, L is

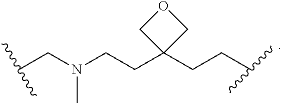 [SMILES: *CCC1(CCN(C)C*)COC1]

In some embodiments, L is

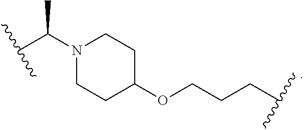 [SMILES: *CCCOC1CCN([C@@H](*)C)CC1]

In some embodiments, L is

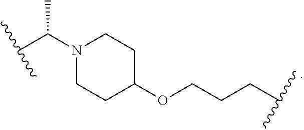 [SMILES: *CCCOC1CCN([C@H](*)C)CC1]

In some embodiments, L is

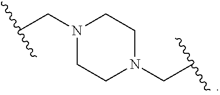 [SMILES: *CN1CCN(C*)CC1]

In some embodiments, L is

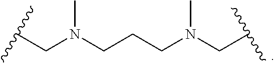 [SMILES: *CN(C)CCCN(C)C*]

In some embodiments, L is

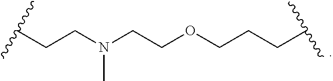 [SMILES: *CCCOCCN(C)CC*]

In some embodiments, L is

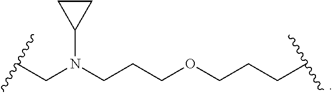 [SMILES: *CCCOCCCN(C*)C1CC1]

In some embodiments, L is

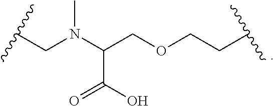 [SMILES: *CCOCC(C(=O)O)N(C)C*]

In some embodiments, L is

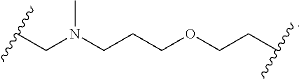 [SMILES: *CCOCCCN(C)C*]

In some embodiments, L is

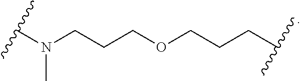 [SMILES: *CCCOCCCN(*)C]

In some embodiments, L is

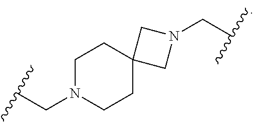 [SMILES: *CN1CCC2(CC1)CN(C*)C2]

In some embodiments, L is

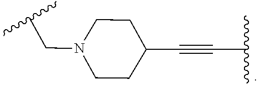 [SMILES: *C#CC1CCN(C*)CC1]

In some embodiments, L is

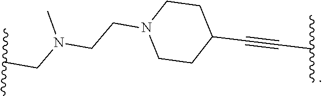 [SMILES: *C#CC1CCN(CCN(C)C*)CC1]

In some embodiments, L is

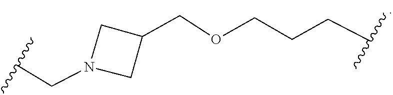 [SMILES: *CCCOCC1CN(C*)C1]

In some embodiments, L is a covalent bond. In some embodiments, L is

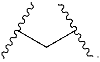 [SMILES: *C*]

In some embodiments, L is

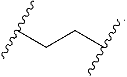 [SMILES: *CC*]

In some embodiments, L is

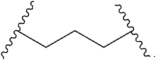 [SMILES: *CCC*]

In some embodiments, L is

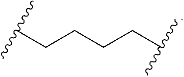 [SMILES: *CCCC*]

In some embodiments, L is

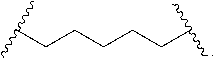 [SMILES: *CCCCC*]

In some embodiments, L is

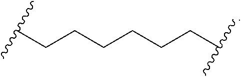 [SMILES: *CCCCCC*]

In some embodiments, L is

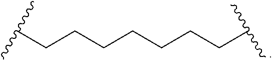 [SMILES: *CCCCCCC*]

In some embodiments, L is

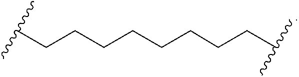 [SMILES: *CCCCCCCC*]

In some embodiments, L is

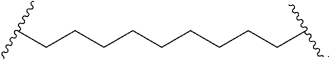 [SMILES: *CCCCCCCCC*]

In some embodiments, L is a covalent bond. In some embodiments, L is

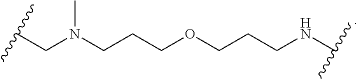 [SMILES: *CN(C)CCCOCCCN*]

In some embodiments, L is

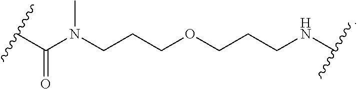 [SMILES: *NCCCOCCCN(C)C(*)=O]

In some embodiments, L is

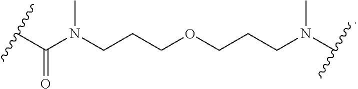 [SMILES: *C(=O)N(C)CCCOCCCN(*)C]

In some embodiments, L is

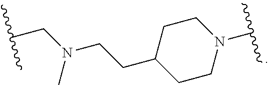 [SMILES: *CN(C)CCC1CCN(*)CC1]

In some embodiments, L is

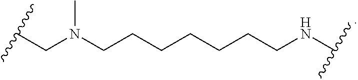 [SMILES: *CN(C)CCCCCCCN*]

In some embodiments, L is

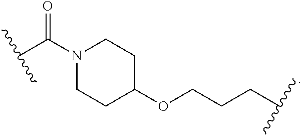 [SMILES: *CCCOC1CCN(C(*)=O)CC1]

In some embodiments, L is

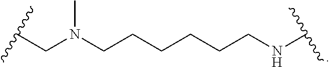 [SMILES: *CN(C)CCCCCCN*]

In some embodiments, L is

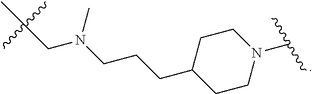 [SMILES: *CN(C)CCCC1CCN(*)CC1]

In some embodiments, L is

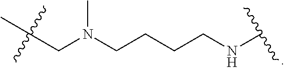 [SMILES: *CN(C)CCCCN*]

In some embodiments, L is

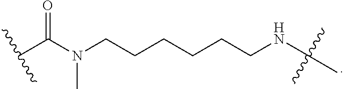 [SMILES: *NCCCCCCN(C)C(*)=O]

In some embodiments, L is

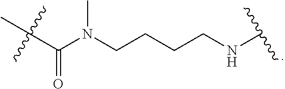 [SMILES: *NCCCCN(C)C(*)=O]

In some embodiments, L is

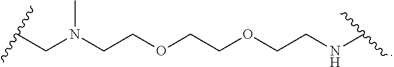 [SMILES: *CN(C)CCOCCOCCN*]

In some embodiments, L is

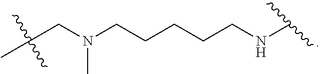 [SMILES: *CN(C)CCCCCN*]

In some embodiments, L is

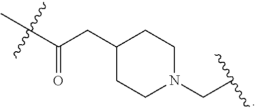 [SMILES: *CN1CCC(CC(*)=O)CC1]

In some embodiments, L is

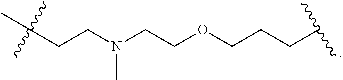 [SMILES: *CCCOCCN(C)CC*]

In some embodiments, L is

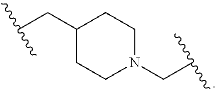 [SMILES: *CC1CCN(C*)CC1]

In some embodiments, L is

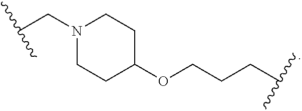 [SMILES: *CCCOC1CCN(C*)CC1]

In some embodiments, L is

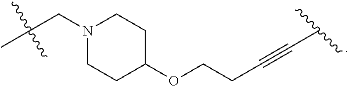 [SMILES: *C#CCCOC1CCN(C*)CC1]

In some embodiments, L is

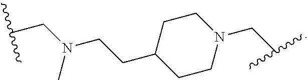 [SMILES: *CN(C)CCC1CCN(C*)CC1]

In some embodiments, L is

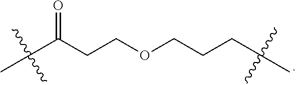 [SMILES: *CCCOCCC(*)=O]

In some embodiments, L is

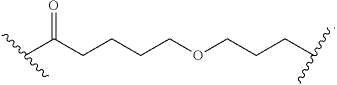 [SMILES: *CCCOCCCCC(*)=O]

In some embodiments, L is

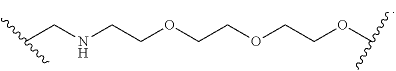 [SMILES: *CNCCOCCOCCO*]

In some embodiments, L is

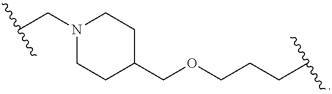 [SMILES: *CCCOCC1CCN(C*)CC1]

In some embodiments, L is

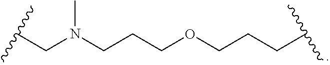 [SMILES: *CCCOCCCN(C)C*]

In some embodiments, L is

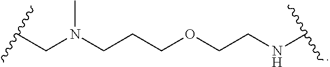 [SMILES: *CN(C)CCCOCCN*]

In some embodiments, L is

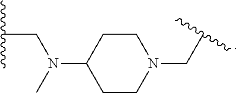 [SMILES: *CN1CCC(N(C)C*)CC1]

In some embodiments, L is

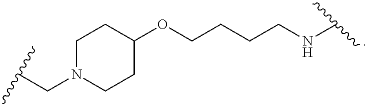 [SMILES: *CN1CCC(OCCCCN*)CC1]

In some embodiments, L is

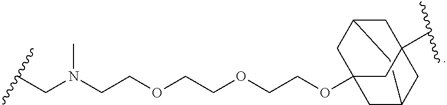 [SMILES: *CN(C)CCOCCOCCOC12CC3CC(CC(*)(C3)C1)C2]

In some embodiments, L is

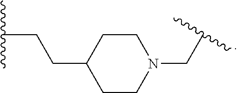 [SMILES: *CCC1CCN(C*)CC1]

In some embodiments, L is

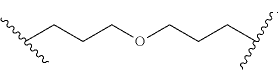 [SMILES: *CCCOCCC*]

In some embodiments, L is

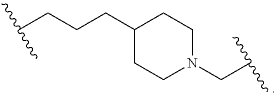 [SMILES: *CCCC1CCN(C*)CC1]

In some embodiments, L is

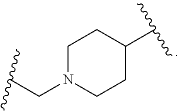 [SMILES: *CN1CCC(*)CC1]

In some embodiments, L is

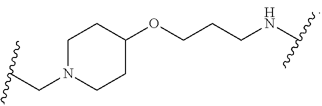 [SMILES: *CN1CCC(OCCCN*)CC1]

In some embodiments, L is

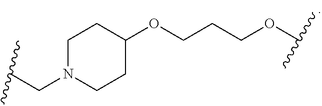 [SMILES: *CN1CCC(OCCCO*)CC1]

In some embodiments, L is

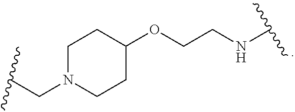 [SMILES: *CN1CCC(OCCN*)CC1]

In some embodiments, L is

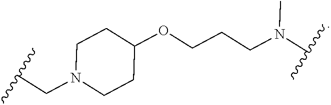 [SMILES: *CN1CCC(OCCCN(*)C)CC1]

In some embodiments, L is

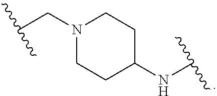 [SMILES: *CN1CCC(N*)CC1]

In some embodiments, L is

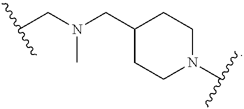 [SMILES: *CN(C)CC1CCN(*)CC1]

In some embodiments, L is

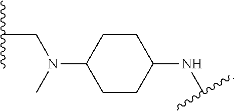 [SMILES: *CN(C)C1CCC(N*)CC1]

In some embodiments, L is

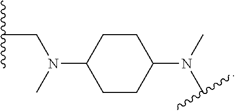 [SMILES: *CN(C)C1CCC(N(*)C)CC1]

In some embodiments, L is

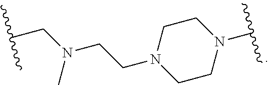 [SMILES: *CN(C)CCN1CCN(*)CC1]

In some embodiments, L is

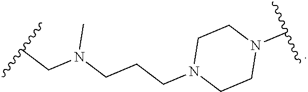 [SMILES: *CN(C)CCCN1CCN(*)CC1]

In some embodiments, L is

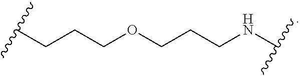 [SMILES: *CCCOCCCN*]

In some embodiments, L is

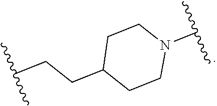 [SMILES: *CCC1CCN(*)CC1]

In some embodiments, L is

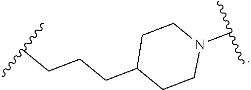 [SMILES: *CCCC1CCN(*)CC1]

In some embodiments, L is

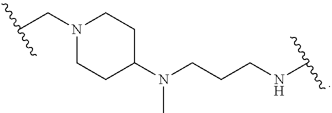 [SMILES: *CN1CCC(N(C)CCCN*)CC1]

In some embodiments, L is

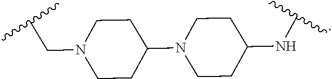 [SMILES: *CN1CCC(N2CCC(N*)CC2)CC1]

In some embodiments, L is

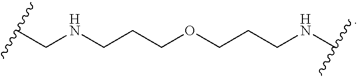 [SMILES: *CNCCCOCCCN*]

In some embodiments, L is

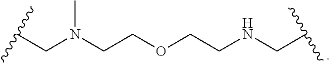 [SMILES: *CNCCOCCN(C)C*]

In some embodiments, Lis

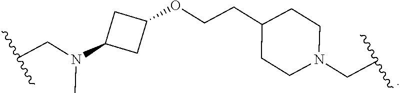 [SMILES: *CN1CCC(CCO[C@H]2C[C@H](N(C)C*)C2)CC1]

In some embodiments, L is

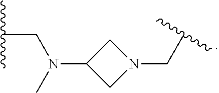 [SMILES: *CN1CC(N(C)C*)C1]

In some embodiments, L is

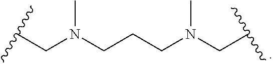 [SMILES: *CN(C)CCCN(C)C*]

In some embodiments, L is

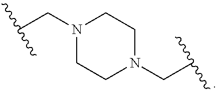 [SMILES: *CN1CCN(C*)CC1]

In some embodiments, L is

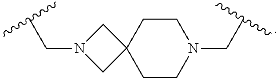 [SMILES: *CN1CCC2(CC1)CN(C*)C2]

In some embodiments, L is

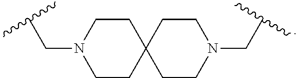 [SMILES: *CN1CCC2(CC1)CCN(C*)CC2]

In some embodiments, L is

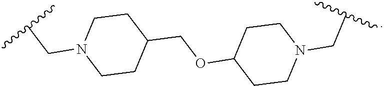 [SMILES: *CN1CCC(COC2CCN(C*)CC2)CC1]

In some embodiments, L is

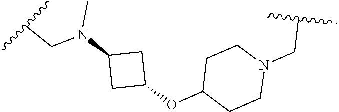 [SMILES: *CN1CCC(O[C@H]2C[C@H](N(C)C*)C2)CC1]

In some embodiments, L is

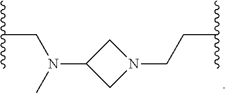 [SMILES: *CCN1CC(N(C)C*)C1]

In some embodiments, L is

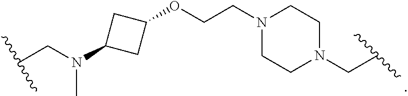 [SMILES: *CN1CCN(CCO[C@H]2C[C@H](N(C)C*)C2)CC1]

In some embodiments, L is

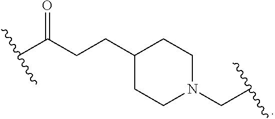 [SMILES: *CN1CCC(CCC(*)=O)CC1]

In some embodiments, L is

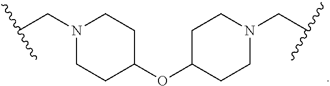 [SMILES: *CN1CCC(OC2CCN(C*)CC2)CC1]

In some embodiments, L is

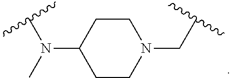 [SMILES: *CN1CCC(N(*)C)CC1]

In some embodiments, L is

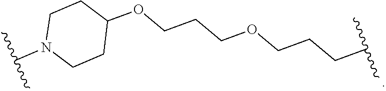 [SMILES: *CCCOCCCOC1CCN(*)CC1]

In some embodiments, L is

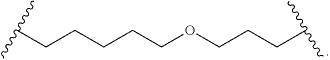 [SMILES: *CCCCCOCCC*]

In some embodiments, L is

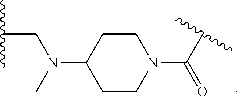 [SMILES: *CN(C)C1CCN(C(*)=O)CC1]

In some embodiments, L is

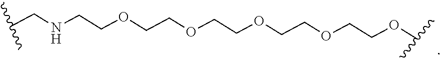 [SMILES: *CNCCOCCOCCOCCOCCO*]

In some embodiments, L is

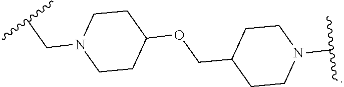 [SMILES: *CN1CCC(OCC2CCN(*)CC2)CC1]

In some embodiments, L is

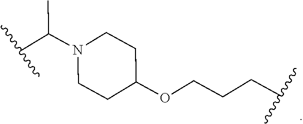 [SMILES: *CCCOC1CCN(C(*)C)CC1]

In some embodiments, L is

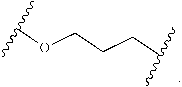 [SMILES: *CCCO*]

In some embodiments, L is

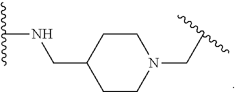 [SMILES: *CN1CCC(CN*)CC1]

In some embodiments, L is

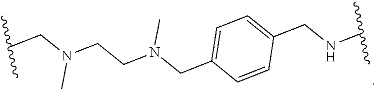 [SMILES: *CN(C)CCN(C)Cc1ccc(CN*)cc1]

In some embodiments, L is

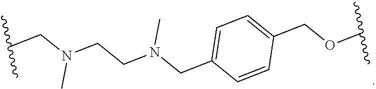 [SMILES: *CN(C)CCN(C)Cc1ccc(CO*)cc1]

In some embodiments, L is

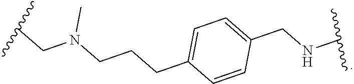 [SMILES: *CN(C)CCCc1ccc(CN*)cc1]

In some embodiments, L is

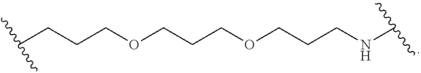 [SMILES: *CCCOCCCOCCCN*]

In some embodiments, L is

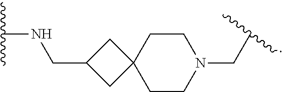 [SMILES: *CN1CCC2(CC1)CC(CN*)C2]

In some embodiments, L is

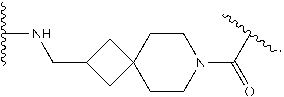 [SMILES: *NCC1CC2(CCN(C(*)=O)CC2)C1]

In some embodiments, L is

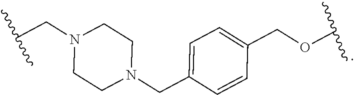 [SMILES: *CN1CCN(Cc2ccc(CO*)cc2)CC1]

In some embodiments, L is

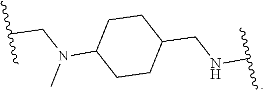 [SMILES: *CN(C)C1CCC(CN*)CC1]

In some embodiments, L is

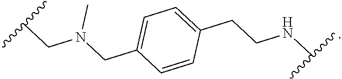 [SMILES: *CN(C)Cc1ccc(CCN*)cc1]

In some embodiments, L is

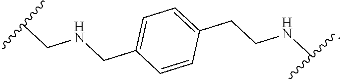 [SMILES: *CNCc1ccc(CCN*)cc1]

In some embodiments, L is

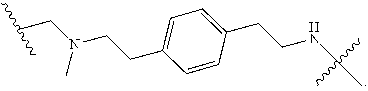 [SMILES: *CN(C)CCc1ccc(CCN*)cc1]

In some embodiments, L is

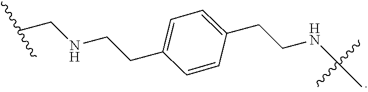 [SMILES: *CNCCc1ccc(CCN*)cc1]

In some embodiments, L is

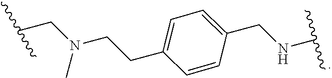 [SMILES: *CN(C)CCc1ccc(CN*)cc1]

In some embodiments, L is

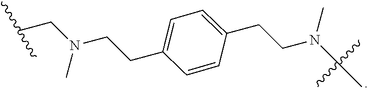 [SMILES: *CN(C)CCc1ccc(CCN(*)C)cc1]

In some embodiments, L is

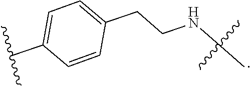 [SMILES: *NCCc1ccc(*)cc1]

In some embodiments, L is

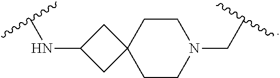 [SMILES: *CN1CCC2(CC1)CC(N*)C2]

In some embodiments, L is

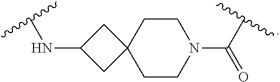 [SMILES: *NC1CC2(CCN(C(*)=O)CC2)C1]

In some embodiments, L is

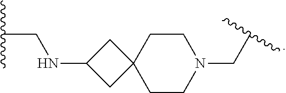 [SMILES: *CNC1CC2(CCN(C*)CC2)C1]

In some embodiments, L is

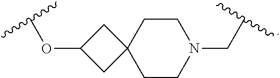 [SMILES: *CN1CCC2(CC1)CC(O*)C2]

In some embodiments, L is

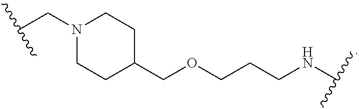 [SMILES: *CN1CCC(COCCCN*)CC1]

In some embodiments, L is

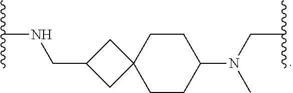 [SMILES: *CN(C)C1CCC2(CC1)CC(CN*)C2]

In some embodiments, L is

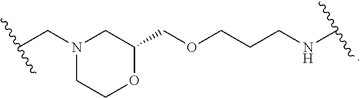 [SMILES: *CN1CCO[C@@H](COCCCN*)C1]

In some embodiments, L is

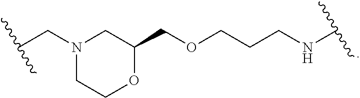 [SMILES: *CN1CCO[C@H](COCCCN*)C1]

In some embodiments, L is

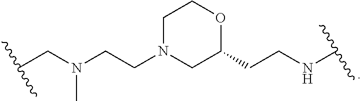 [SMILES: *CN(C)CCN1CCO[C@H](CCN*)C1]

In some embodiments, L is

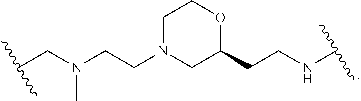 [SMILES: *CN(C)CCN1CCO[C@@H](CCN*)C1]

In some embodiments, L is

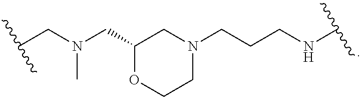 [SMILES: *CN(C)C[C@@H]1CN(CCCN*)CCO1]

In some embodiments, L is

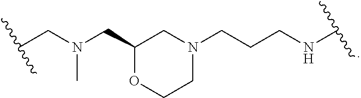 [SMILES: *CN(C)C[C@H]1CN(CCCN*)CCO1]

In some embodiments, L is

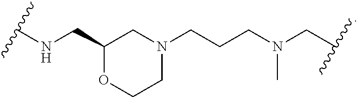 [SMILES: *CN(C)CCCN1CCO[C@@H](CN*)C1]

In some embodiments, L is

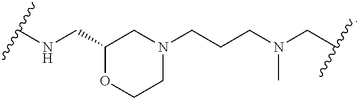 [SMILES: *CN(C)CCCN1CCO[C@H](CN*)C1]

In some embodiments, L is

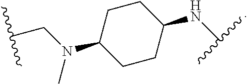 [SMILES: *CN(C)[C@H]1CC[C@@H](N*)CC1]

In some embodiments, L is

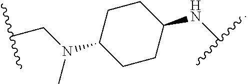 [SMILES: *CN(C)[C@H]1CC[C@H](N*)CC1]

In some embodiments, L is

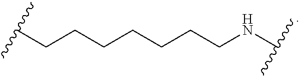 [SMILES: *CCCCCCCN*]

In some embodiments, L is

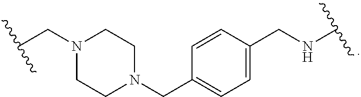 [SMILES: *CN1CCN(Cc2ccc(CN*)cc2)CC1]

In some embodiments, L is

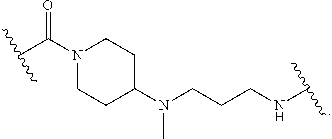 [SMILES: *NCCCN(C)C1CCN(C(*)=O)CC1]

In some embodiments, L is

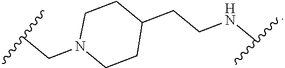 [SMILES: *CN1CCC(CCN*)CC1]

In some embodiments, L is

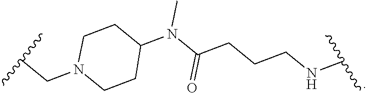 [SMILES: *CN1CCC(N(C)C(=O)CCCN*)CC1]

In some embodiments, L is

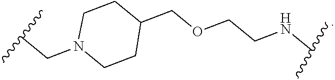 [SMILES: *CN1CCC(COCCN*)CC1]

In some embodiments, L is

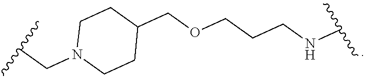 [SMILES: *CN1CCC(COCCCN*)CC1]

In some embodiments, L is

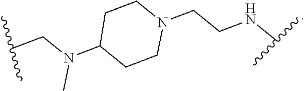 [SMILES: *CN(C)C1CCN(CCN*)CC1]

In some embodiments, L is

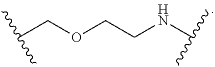 [SMILES: *COCCN*]

In some embodiments, L is

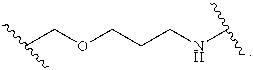 [SMILES: *COCCCN*]

In some embodiments, L is

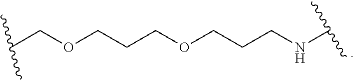 [SMILES: *COCCCOCCCN*]

In some embodiments, L is

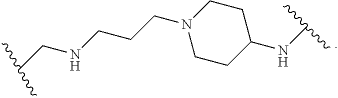 [SMILES: *CNCCCN1CCC(N*)CC1]

In some embodiments, L is

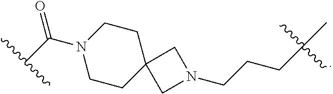 [SMILES: *CCCN1CC2(CCN(C(*)=O)CC2)C1]

In some embodiments, L is

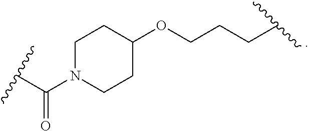 [SMILES: *CCCOC1CCN(C(*)=O)CC1]

In some embodiments, L is

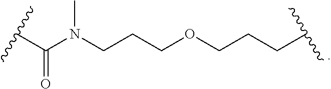 [SMILES: *CCCOCCCN(C)C(*)=O]

In some embodiments, L is

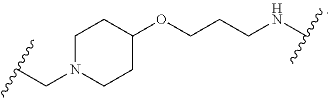 [SMILES: *CN1CCC(OCCCN*)CC1]

In some embodiments, L is

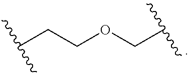 [SMILES: *CCOC*]

In some embodiments, L is

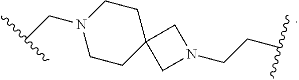 [SMILES: *CCN1CC2(CCN(C*)CC2)C1]

In some embodiments, L is

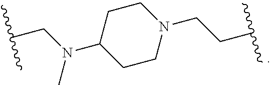 [SMILES: *CCN1CCC(N(C)C*)CC1]

In some embodiments, L is

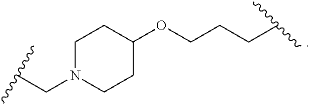 [SMILES: *CCCOC1CCN(C*)CC1]

In some embodiments, L is

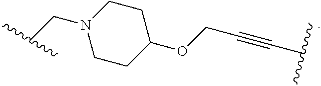 [SMILES: *C#CCOC1CCN(C*)CC1]

In some embodiments, L is

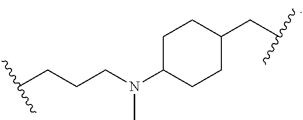 [SMILES: *CCCN(C)C1CCC(C*)CC1]

In some embodiments, L is

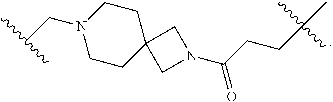 [SMILES: *CCC(=O)N1CC2(CCN(C*)CC2)C1]

In some embodiments, L is

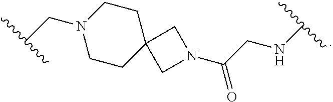 [SMILES: *CN1CCC2(CC1)CN(C(=O)CN*)C2]

In some embodiments, L is

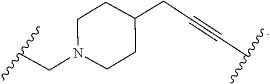 [SMILES: *C#CCC1CCN(C*)CC1]

In some embodiments, L is

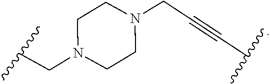 [SMILES: *C#CCN1CCN(C*)CC1]

In some embodiments, L is

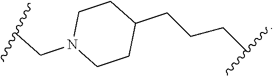 [SMILES: *CCCC1CCN(C*)CC1]

In some embodiments, L is

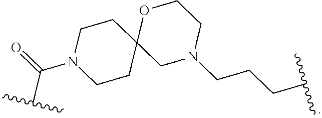 [SMILES: *CCCN1CCOC2(CCN(C(*)=O)CC2)C1]

In some embodiments, L is

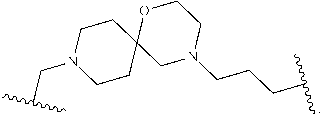 [SMILES: *CCCN1CCOC2(CCN(C*)CC2)C1]

In some embodiments, L is

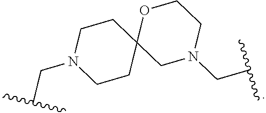 [SMILES: *CN1CCC2(CC1)CN(C*)CCO2]

In some embodiments, L is

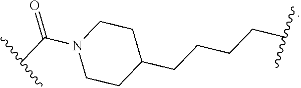 [SMILES: *CCCCC1CCN(C(*)=O)CC1]

In some embodiments, L is

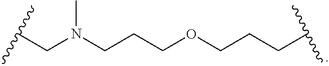 [SMILES: *CCCOCCCN(C)C*]

In some embodiments, L is

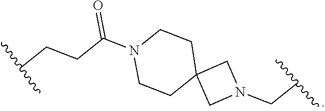 [SMILES: *CCC(=O)N1CCC2(CC1)CN(C*)C2]

In some embodiments, L is

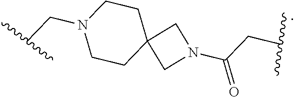 [SMILES: *CC(=O)N1CC2(CCN(C*)CC2)C1]

In some embodiments, L is

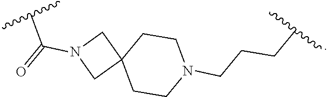 [SMILES: *CCCN1CCC2(CC1)CN(C(*)=O)C2]

In some embodiments, L is

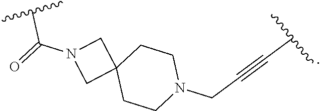 [SMILES: *C#CCN1CCC2(CC1)CN(C(*)=O)C2]

In some embodiments, L is

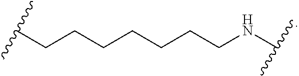 [SMILES: *CCCCCCCN*]

In some embodiments, L is

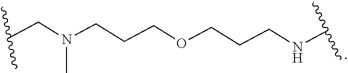 [SMILES: *CN(C)CCCOCCCN*]

In some embodiments, L is

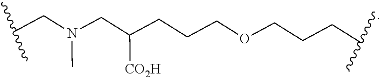 [SMILES: *CCCOCCCC(CN(C)C*)C(=O)O]

In some embodiments, L is

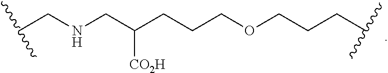 [SMILES: *CCCOCCCC(CNC*)C(=O)O]

In some embodiments, L is

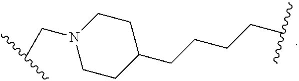 [SMILES: *CCCCC1CCN(C*)CC1]

In some embodiments, L is

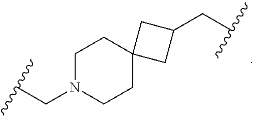 [SMILES: *CC1CC2(CCN(C*)CC2)C1]

In some embodiments, L is

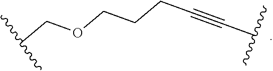 [SMILES: *C#CCCCOC*]

In some embodiments, L is

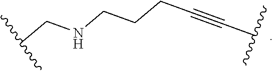 [SMILES: *C#CCCCNC*]

In some embodiments, L is

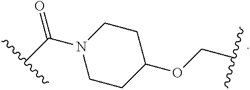 [SMILES: *COC1CCN(C(*)=O)CC1]

In some embodiments, L is

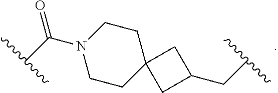 [SMILES: *CC1CC2(CCN(C(*)=O)CC2)C1]

In some embodiments, L is

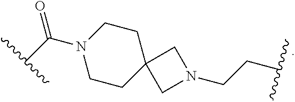 [SMILES: *CCN1CC2(CCN(C(*)=O)CC2)C1]

In some embodiments, L is

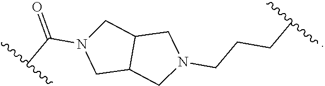 [SMILES: *CCCN1CC2CN(C(*)=O)CC2C1]

In some embodiments, L is

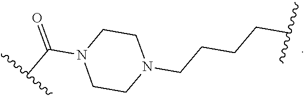 [SMILES: *CCCCN1CCN(C(*)=O)CC1]

In some embodiments, L is

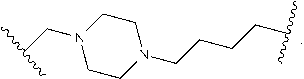 [SMILES: *CCCCN1CCN(C*)CC1]

In some embodiments, L is

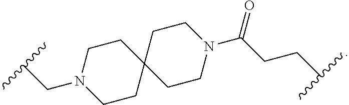 [SMILES: *CCC(=O)N1CCC2(CCN(C*)CC2)CC1]

In some embodiments, L is

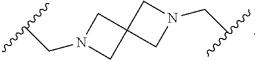 [SMILES: *CN1CC2(C1)CN(C*)C2]

In some embodiments, L is

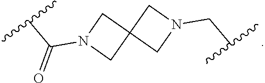 [SMILES: *CN1CC2(C1)CN(C(*)=O)C2]

In some embodiments, L is

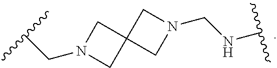 [SMILES: *CN1CC2(C1)CN(CN*)C2]

In some embodiments, L is

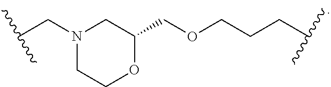 [SMILES: *CCCOC[C@H]1CN(C*)CCO1]

In some embodiments, L is

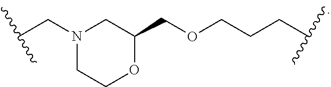 [SMILES: *CCCOC[C@@H]1CN(C*)CCO1]

In some embodiments, L is

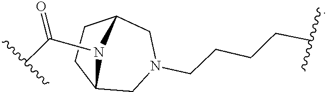 [SMILES: *CCCCN1C[C@H]2CC[C@@H](C1)N2C(*)=O]

In some embodiments, L is

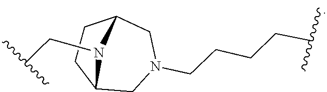 [SMILES: *CCCCN1C[C@H]2CC[C@@H](C1)N2C*]

In some embodiments, L is

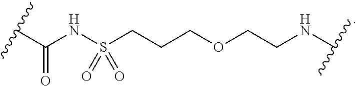 [SMILES: *NCCOCCCS(=O)(=O)NC(*)=O]

In some embodiments, L is

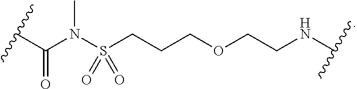 [SMILES: *NCCOCCCS(=O)(=O)N(C)C(*)=O]

In some embodiments, L is

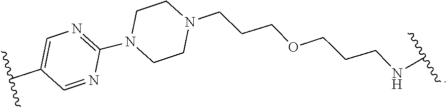 [SMILES: *NCCCOCCCN1CCN(c2ncc(*)cn2)CC1]

In some embodiments, L is

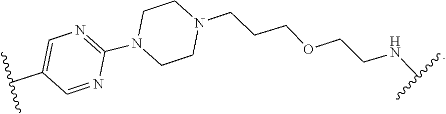 [SMILES: *NCCOCCCN1CCN(c2ncc(*)cn2)CC1]

In some embodiments, L is

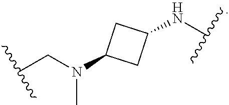 [SMILES: *CN(C)[C@H]1C[C@H](N*)C1]

In some embodiments, L is

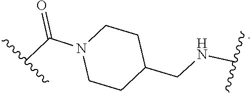 [SMILES: *NCC1CCN(C(*)=O)CC1]

In some embodiments, L is

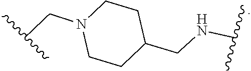 [SMILES: *CN1CCC(CN*)CC1]

In some embodiments, L is

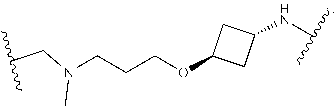 [SMILES: *CN(C)CCCO[C@H]1C[C@H](N*)C1]

In some embodiments, L is

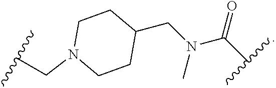 [SMILES: *CN1CCC(CN(C)C(*)=O)CC1]

In some embodiments, L is

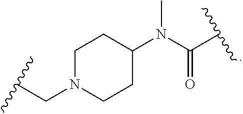 [SMILES: *CN1CCC(N(C)C(*)=O)CC1]

In some embodiments, L is

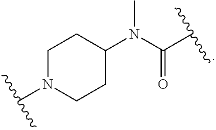 [SMILES: *C(=O)N(C)C1CCN(*)CC1]

In some embodiments, L is

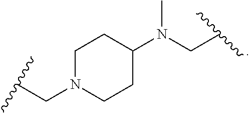 [SMILES: *CN1CCC(N(C)C*)CC1]

In some embodiments, L is

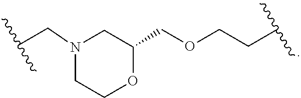 [SMILES: *CCOC[C@H]1CN(C*)CCO1]

In some embodiments, L is

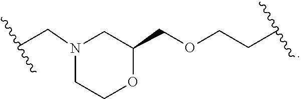 [SMILES: *CCOC[C@@H]1CN(C*)CCO1]

In some embodiments, L is

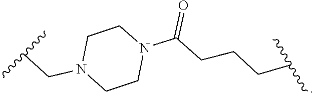 [SMILES: *CCCC(=O)N1CCN(C*)CC1]

In some embodiments, L is

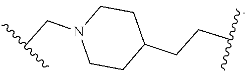 [SMILES: *CCC1CCN(C*)CC1]

In some embodiments, L is

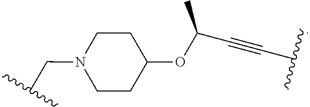 [SMILES: *C#C[C@H](C)OC1CCN(C*)CC1]

In some embodiments, L is

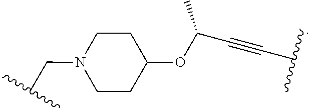 [SMILES: *C#C[C@@H](C)OC1CCN(C*)CC1]

In some embodiments, L is

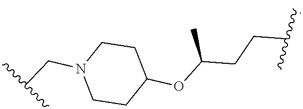 [SMILES: *CC[C@H](C)OC1CCN(C*)CC1]

In some embodiments, L is

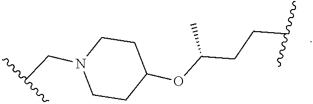 [SMILES: *CC[C@@H](C)OC1CCN(C*)CC1]

In some embodiments, L is

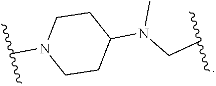 [SMILES: *CN(C)C1CCN(*)CC1]

In some embodiments, Lis

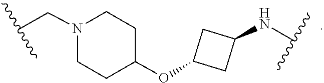 [SMILES: *CN1CCC(O[C@H]2C[C@H](N*)C2)CC1]

In some embodiments, L is

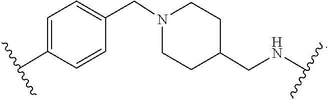 [SMILES: *NCC1CCN(Cc2ccc(*)cc2)CC1]

In some embodiments, L is

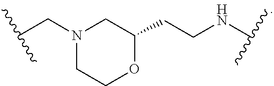 [SMILES: *CN1CCO[C@@H](CCN*)C1]

In some embodiments, L is

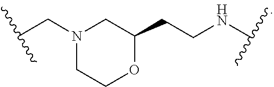 [SMILES: *CN1CCO[C@H](CCN*)C1]

In some embodiments, L is

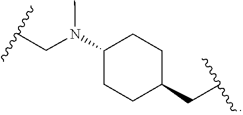 [SMILES: *CN(C)[C@H]1CC[C@H](C*)CC1]

In some embodiments, L is

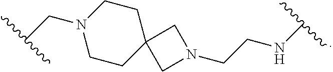 [SMILES: *CN1CCC2(CC1)CN(CCN*)C2]

In some embodiments, L is

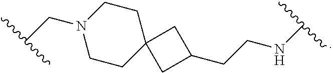 [SMILES: *CN1CCC2(CC1)CC(CCN*)C2]

In some embodiments, L is

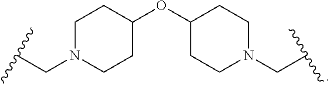 [SMILES: *CN1CCC(OC2CCN(C*)CC2)CC1]

In some embodiments, L is

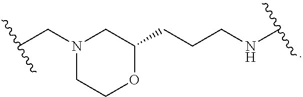 [SMILES: *CN1CCO[C@@H](CCCN*)C1]

In some embodiments, L is

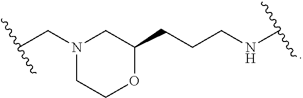 [SMILES: *CN1CCO[C@H](CCCN*)C1]

In some embodiments, L is

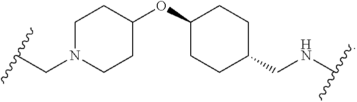 [SMILES: *CN1CCC(O[C@H]2CC[C@H](CN*)CC2)CC1]

In some embodiments, L is

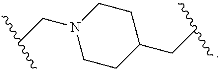 [SMILES: *CC1CCN(C*)CC1]

In some embodiments, L is

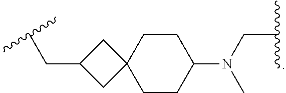 [SMILES: *CC1CC2(CCC(N(C)C*)CC2)C1]

In some embodiments, L is

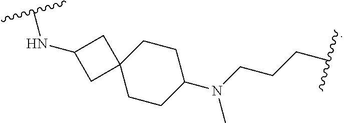 [SMILES: *CCCN(C)C1CCC2(CC1)CC(N*)C2]

In some embodiments, L is

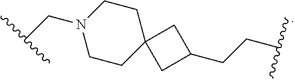 [SMILES: *CCC1CC2(CCN(C*)CC2)C1]

In some embodiments, L is

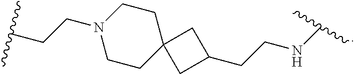 [SMILES: *CCN1CCC2(CC1)CC(CCN*)C2]

In some embodiments, L is

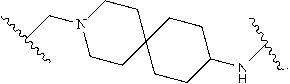 [SMILES: *CN1CCC2(CCC(N*)CC2)CC1]

In some embodiments, L is

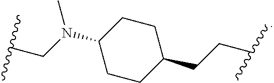 [SMILES: *CC[C@H]1CC[C@H](N(C)C*)CC1]

In some embodiments, L is

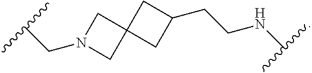 [SMILES: *CN1CC2(CC(CCN*)C2)C1]

In some embodiments, L is

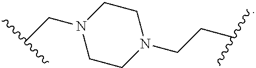 [SMILES: *CCN1CCN(C*)CC1]

In some embodiments, L is

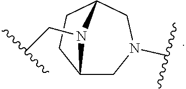 [SMILES: *CN1[C@@H]2CC[C@H]1CN(*)C2 ; "."]

In some embodiments, L is

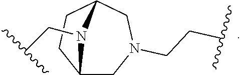 [SMILES: *CCN1C[C@H]2CC[C@@H](C1)N2C* ; "."]

In some embodiments, L is

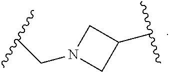 [SMILES: *CN1CC(*)C1 ; "."]

In some embodiments, L is

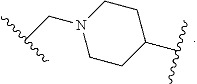 [SMILES: *CN1CCC(*)CC1 ; "."]

In some embodiments, L is

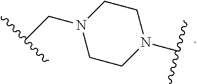 [SMILES: *CN1CCN(*)CC1 ; "."]

In some embodiments, L is

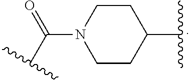 [SMILES: *C(=O)N1CCC(*)CC1]

In some embodiments, L is

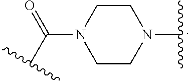 [SMILES: *C(=O)N1CCN(*)CC1]

In some embodiments, L is

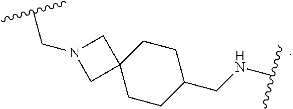 [SMILES: *CN1CC2(CCC(CN*)CC2)C1 ; "."]

In some embodiments, L is

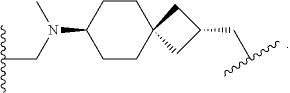

In some embodiments, L is

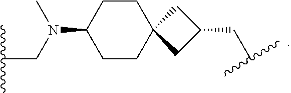

In some embodiments, L is

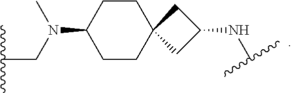

In some embodiments, L is

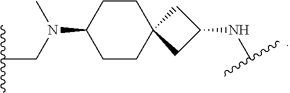

In some embodiments, L is

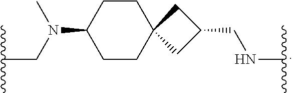

In some embodiments, L is

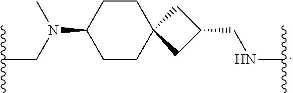

In some embodiments, L is

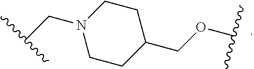 [SMILES: *CN1CCC(CO*)CC1 ; "."]

In some embodiments, L is

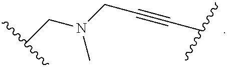 [SMILES: *C#CCN(C)C*]

In some embodiments, L is

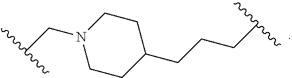 [SMILES: *CCCC1CCN(C*)CC1]

In some embodiments, L is

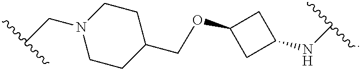 [SMILES: *CN1CCC(CO[C@H]2C[C@H](N*)C2)CC1]

In some embodiments, L is

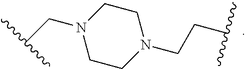 [SMILES: *CCN1CCN(C*)CC1]

In some embodiments, L is

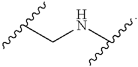 [SMILES: *CN*]

In some embodiments, L is

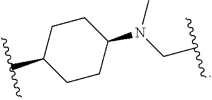 [SMILES: *CN(C)[C@H]1CC[C@@H](*)CC1]

In some embodiments, L is

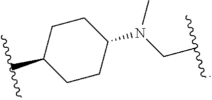 [SMILES: *CN(C)[C@H]1CC[C@H](*)CC1]

In some embodiments, L is

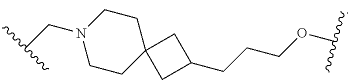 [SMILES: *CN1CCC2(CC1)CC(CCCO*)C2]

In some embodiments, L is

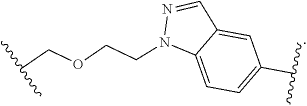 [SMILES: *COCCn1ncc2cc(*)ccc21]

In some embodiments, L is

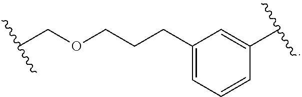 [SMILES: *COCCCc1cccc(*)c1]

In some embodiments, L is

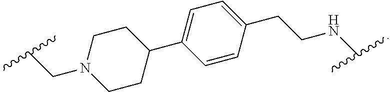 [SMILES: *CN1CCC(c2ccc(CCN*)cc2)CC1]

In some embodiments, L is

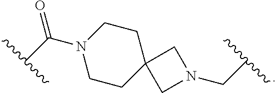 [SMILES: *CN1CC2(CCN(C(*)=O)CC2)C1]

In some embodiments, L is

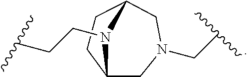 [SMILES: *CCN1[C@@H]2CC[C@H]1CN(C*)C2]

In some embodiments, L is

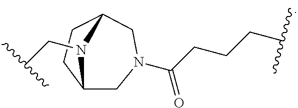 [SMILES: *CCCC(=O)N1C[C@H]2CC[C@@H](C1)N2C*]

In some embodiments, L is

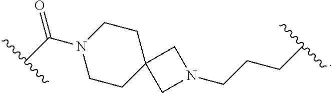 [SMILES: *CCCN1CC2(CCN(C(*)=O)CC2)C1]

In some embodiments, L is

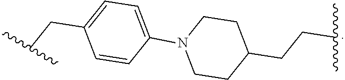 [SMILES: *CCC1CCN(c2ccc(C*)cc2)CC1]

In some embodiments, L is

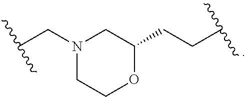 [SMILES: *CC[C@H]1CN(C*)CCO1]

In some embodiments, L is

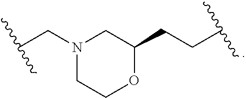 [SMILES: *CC[C@@H]1CN(C*)CCO1]

In some embodiments, L is

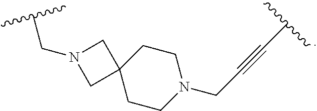 [SMILES: *C#CCN1CCC2(CC1)CN(C*)C2]

In some embodiments, L is

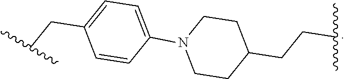 [SMILES: *CCC1CCN(c2ccc(C*)cc2)CC1]

In some embodiments, L is

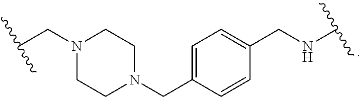 [SMILES: *CN1CCN(Cc2ccc(CN*)cc2)CC1]

In some embodiments, L is

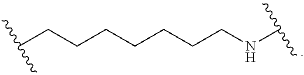 [SMILES: *CCCCCCCN*]

In some embodiments, L is

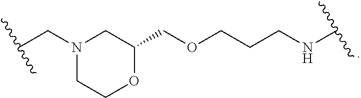 [SMILES: *CN1CCO[C@@H](COCCCN*)C1]

In some embodiments, L is

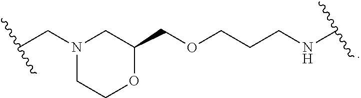 [SMILES: *CN1CCO[C@H](COCCCN*)C1]

In some embodiments, L is

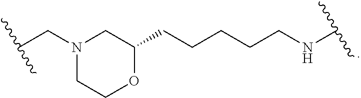 [SMILES: *CN1CCO[C@@H](CCCCCN*)C1]

In some embodiments, L is

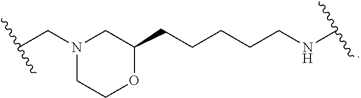 [SMILES: *CN1CCO[C@H](CCCCCN*)C1]

In some embodiments, L is

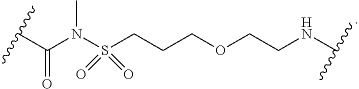 [SMILES: *NCCOCCCS(=O)(=O)N(C)C(*)=O]

In some embodiments, L is

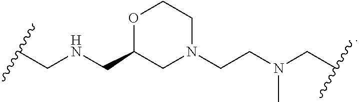 [SMILES: *CNC[C@@H]1CN(CCN(C)C*)CCO1]

In some embodiments, L is

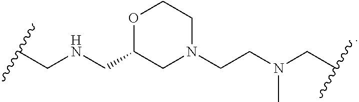 [SMILES: *CNC[C@H]1CN(CCN(C)C*)CCO1]

In some embodiments, L is

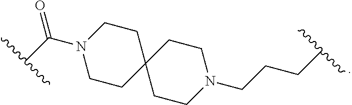 [SMILES: *CCCN1CCC2(CC1)CCN(C(*)=O)CC2]

In some embodiments, L is

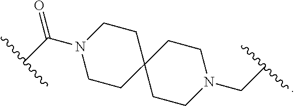 [SMILES: *CN1CCC2(CC1)CCN(C(*)=O)CC2]

In some embodiments, L is

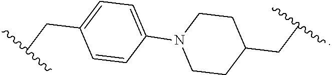 [SMILES: *Cc1ccc(N2CCC(C*)CC2)cc1]

In some embodiments, L is

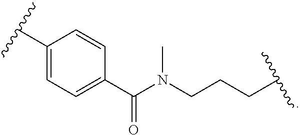 [SMILES: *CCCN(C)C(=O)c1ccc(*)cc1]

In some embodiments, L is

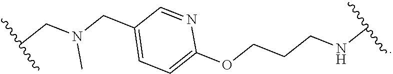 [SMILES: *CN(C)Cc1ccc(OCCCN*)nc1]

In some embodiments, L is

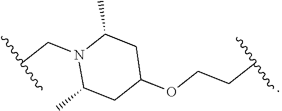 [SMILES: *CCOC1C[C@@H](C)N(C*)[C@@H](C)C1]

In some embodiments, L is

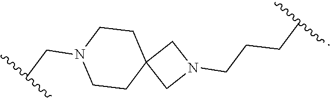 [SMILES: *CCCN1CC2(CCN(C*)CC2)C1]

In some embodiments, L is

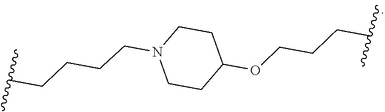 [SMILES: *CCCCN1CCC(OCCC*)CC1]

In some embodiments, L is

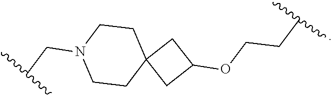 [SMILES: *CCOC1CC2(CCN(C*)CC2)C1]

In some embodiments, L is

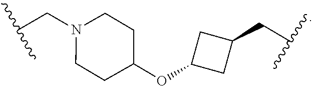 [SMILES: *CN1CCC(O[C@H]2C[C@H](C*)C2)CC1]

In some embodiments, L is

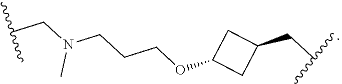 [SMILES: *CN(C)CCCO[C@H]1C[C@H](C*)C1]

In some embodiments, L is

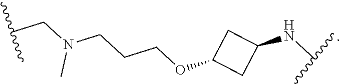 [SMILES: *CN(C)CCCO[C@H]1C[C@H](N*)C1]

In some embodiments, L is

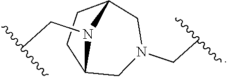 [SMILES: *CN1C[C@H]2CC[C@@H](C1)N2C*]

In some embodiments, L is

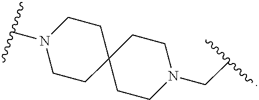 [SMILES: *CN1CCC2(CCN(*)CC2)CC1]

In some embodiments, L is

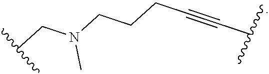 [SMILES: *C#CCCCN(C)C*]

In some embodiments, L is

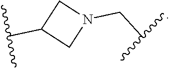 [SMILES: *CN1CC(*)C1]

In some embodiments, L is

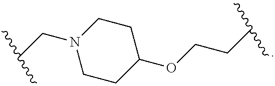 [SMILES: *CCOC1CCN(C*)CC1]

In some embodiments, L is

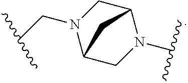 [SMILES: *CN1C[C@H]2C[C@@H]1CN2*]

In some embodiments, L is

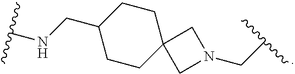 [SMILES: *CN1CC2(CCC(CN*)CC2)C1]

In some embodiments, L is

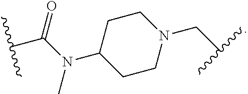 [SMILES: *CN1CCC(N(C)C(*)=O)CC1]

In some embodiments, L is

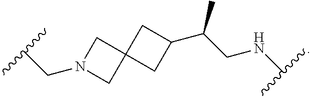 [SMILES: *CN1CC2(CC([C@@H](C)CN*)C2)C1]

In some embodiments, L is

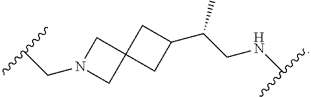 [SMILES: *CN1CC2(CC([C@H](C)CN*)C2)C1]

In some embodiments, L is

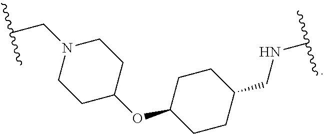 [SMILES: *CN1CCC(O[C@H]2CC[C@H](CN*)CC2)CC1]

In some embodiments, L is

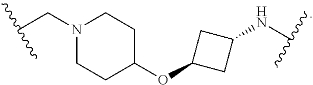 [SMILES: *CN1CCC(O[C@H]2C[C@H](N*)C2)CC1]

In some embodiments, L is

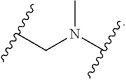 [SMILES: *CN(*)C]

In some embodiments, L is

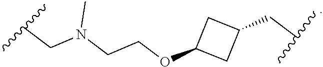 [SMILES: *CN(C)CCO[C@H]1C[C@H](C*)C1]

In some embodiments, L is

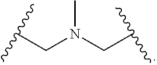 [SMILES: *CN(C)C*]

In some embodiments, L is

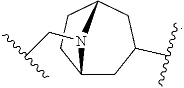 [SMILES: *CN1[C@@H]2CC[C@H]1CC(*)C2]

In some embodiments, L is

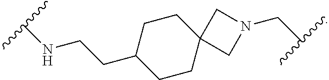 [SMILES: *CN1CC2(CCC(CCN*)CC2)C1]

In some embodiments, L is

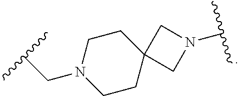 [SMILES: *CN1CCC2(CC1)CN(*)C2]

In some embodiments, L is

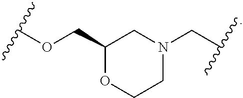 [SMILES: *CN1CCO[C@@H](CO*)C1]

In some embodiments, L is

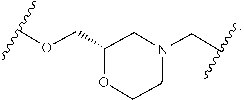 [SMILES: *CN1CCO[C@H](CO*)C1]

In some embodiments, L is

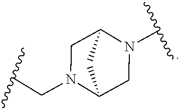 [SMILES: *CN1C[C@@H]2C[C@H]1CN2*]

In some embodiments, L is

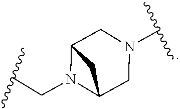 [SMILES: *CN1[C@@H]2C[C@H]1CN(*)C2]

In some embodiments, L is

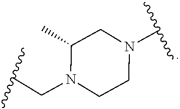 [SMILES: *CN1CCN(*)C[C@H]1C]

In some embodiments, L is

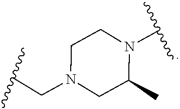 [SMILES: *CN1CCN(*)[C@@H](C)C1]

In some embodiments, L is

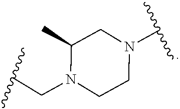 [SMILES: *CN1CCN(*)C[C@@H]1C]

In some embodiments, L is

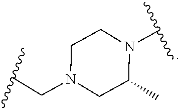 [SMILES: *CN1CCN(*)[C@H](C)C1]

In some embodiments, L is

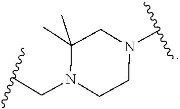 [SMILES: *CN1CCN(*)CC1(C)C]

In some embodiments, L is

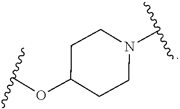 [SMILES: *OC1CCN(*)CC1]

In some embodiments, L is

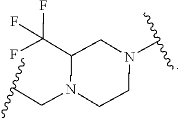 [SMILES: *CN1CCN(*)CC1C(F)(F)F]

In some embodiments, L is

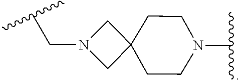 [SMILES: *CN1CC2(CCN(*)CC2)C1]

In some embodiments, L is

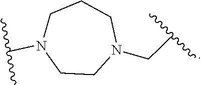 [SMILES: *CN1CCCN(*)CC1]

In some embodiments, L is

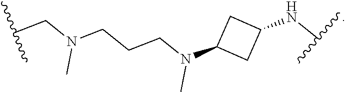 [SMILES: *CN(C)CCCN(C)[C@H]1C[C@H](N*)C1]

In some embodiments, L is

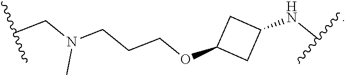 [SMILES: *CN(C)CCCO[C@H]1C[C@H](N*)C1]

In some embodiments, L is

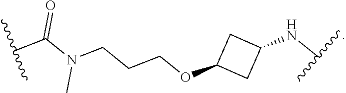 [SMILES: *N[C@H]1C[C@H](OCCCN(C)C(*)=O)C1]

In some embodiments, L is

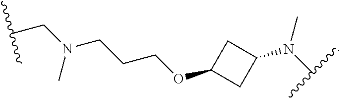 [SMILES: *CN(C)CCCO[C@H]1C[C@H](N(*)C)C1]

In some embodiments, L is

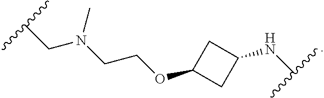 [SMILES: *CN(C)CCO[C@H]1C[C@H](N*)C1]

In some embodiments, L is

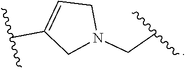 [SMILES: *CN1CC=C(*)C1]

In some embodiments, L is

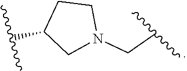 [SMILES: *CN1CC[C@@H](*)C1]

In some embodiments, L is

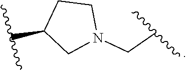 [SMILES: *CN1CC[C@H](*)C1]

In some embodiments, L is

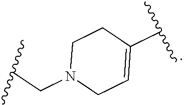 [SMILES: *CN1CC=C(*)CC1]

In some embodiments, L is

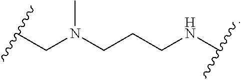 [SMILES: *CN(C)CCCN*]

In some embodiments, L is

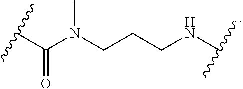 [SMILES: *NCCCN(C)C(*)=O]

In some embodiments, L is

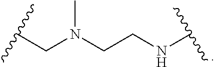 [SMILES: *CN(C)CCN*]

In some embodiments, L is

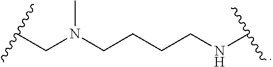 [SMILES: *CN(C)CCCCN*]

In some embodiments, L is

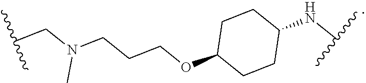 [SMILES: *CN(C)CCCO[C@H]1CC[C@H](N*)CC1]

In some embodiments, L is

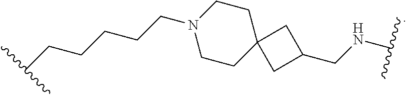 [SMILES: *CCCCCN1CCC2(CC1)CC(CN*)C2]

In some embodiments, L is

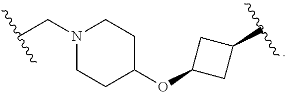 [SMILES: *CN1CCC(O[C@H]2C[C@@H](*)C2)CC1]

In some embodiments, L is

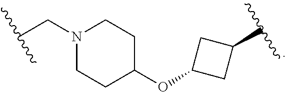 [SMILES: *CN1CCC(O[C@H]2C[C@H](*)C2)CC1]

In some embodiments, L is

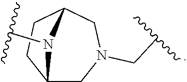 [SMILES: *CN1C[C@H]2CC[C@@H](C1)N2*]

In some embodiments, L is selected from those depicted in Table 1, below.

Without limitation, the point of attachment of L to IRAK and DIM can be, for example when L is

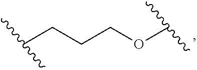 [SMILES: *CCCO*]

either

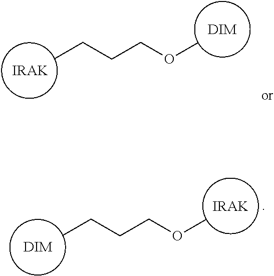

Exemplary compounds of the invention are set forth in Table 1, below.

TABLE 1
Exemplary Compounds

| I-# | Structure |
|---|---|
| I-1 | 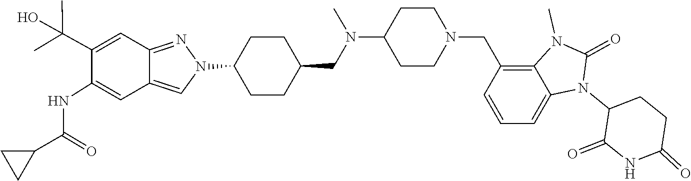 [SMILES: CN(C[C@H]1CC[C@H](n2cc3cc(NC(=O)C4CC4)c(C(C)(C)O)cc3n2)CC1)C1CCN(Cc2cccc3c2n(C)c(=O)n3C2CCC(=O)NC2=O)CC1] |
| I-2 | 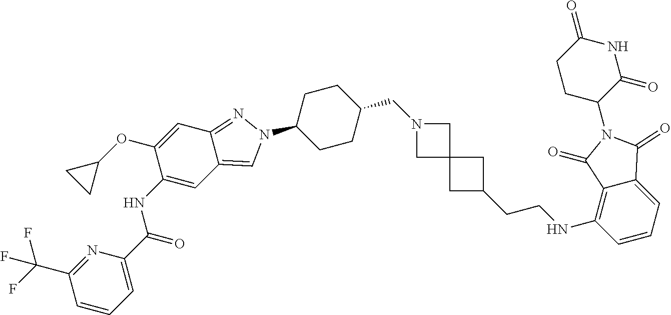 [SMILES: O=C1CCC(N2C(=O)c3cccc(NCCC4CC5(C4)CN(C[C@H]4CC[C@H](n6cc7cc(NC(=O)c8cccc(C(F)(F)F)n8)c(OC8CC8)cc7n6)CC4)C5)c3C2=O)C(=O)N1] |
| I-3 | 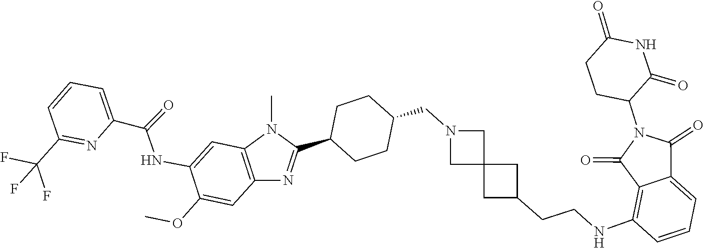 [SMILES: COc1cc2nc([C@H]3CC[C@H](CN4CC5(CC(CCNc6cccc7c6C(=O)N(C6CCC(=O)NC6=O)C7=O)C5)C4)CC3)n(C)c2cc1NC(=O)c1cccc(C(F)(F)F)n1] |
| I-4 | 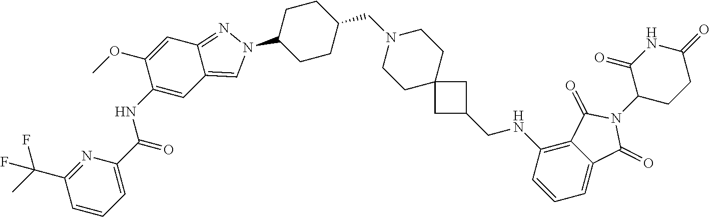 |
| I-5 | 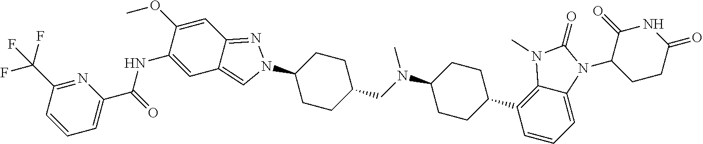 [SMILES: COc1cc2nn([C@H]3CC[C@H](CN(C)[C@H]4CC[C@H](c5cccc6c5n(C)c(=O)n6C5CCC(=O)NC5=O)CC4)CC3)cc2cc1NC(=O)c1cccc(C(F)(F)F)n1] |
| I-6 | 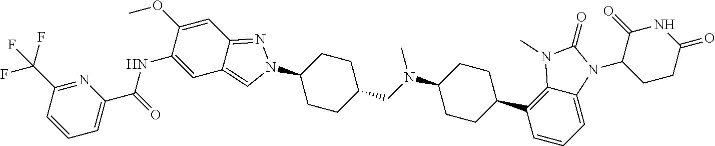 [SMILES: COc1cc2nn([C@H]3CC[C@H](CN(C)[C@H]4CC[C@@H](c5cccc6c5n(C)c(=O)n6C5CCC(=O)NC5=O)CC4)CC3)cc2cc1NC(=O)c1cccc(C(F)(F)F)n1] |
| I-7 | 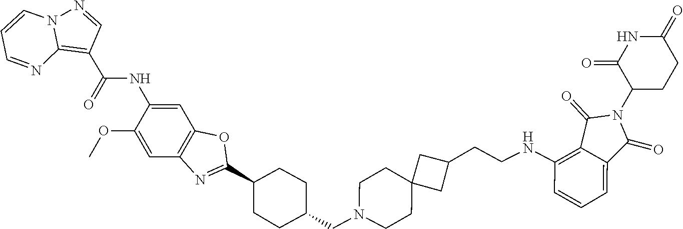 [SMILES: COc1cc2nc([C@H]3CC[C@H](CN4CCC5(CC4)CC(CCNc4cccc6c4C(=O)N(C4CCC(=O)NC4=O)C6=O)C5)CC3)oc2cc1NC(=O)c1cnn2cccnc12] |
| I-8 | 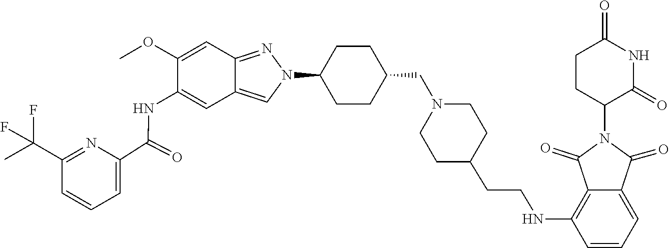 [SMILES: COc1cc2nn([C@H]3CC[C@H](CN4CCC(CCNc5cccc6c5C(=O)N(C5CCC(=O)NC5=O)C6=O)CC4)CC3)cc2cc1NC(=O)c1cccc(C(C)(F)F)n1] |
| I-9 | 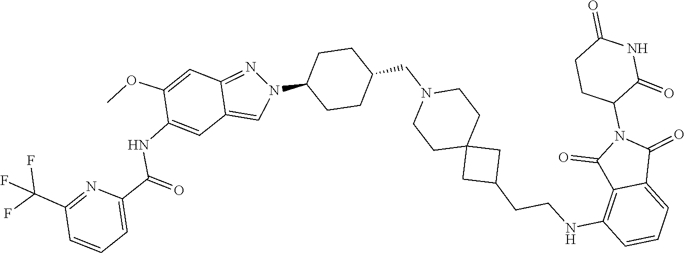 [SMILES: COc1cc2nn([C@H]3CC[C@H](CN4CCC5(CC4)CC(CCNc4cccc6c4C(=O)N(C4CCC(=O)NC4=O)C6=O)C5)CC3)cc2cc1NC(=O)c1cccc(C(F)(F)F)n1] |
| I-10 | 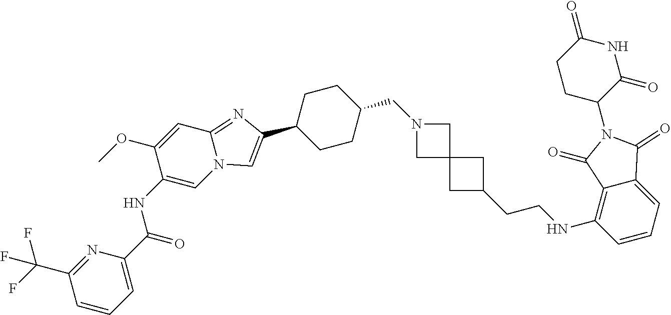 [SMILES: COc1cc2nc([C@H]3CC[C@H](CN4CC5(CC(CCNc6cccc7c6C(=O)N(C6CCC(=O)NC6=O)C7=O)C5)C4)CC3)cn2cc1NC(=O)c1cccc(C(F)(F)F)n1] |
| I-11 | 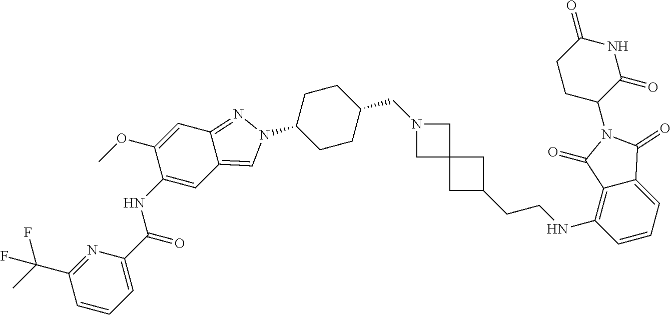 [SMILES: COc1cc2nn([C@H]3CC[C@@H](CN4CC5(CC(CCNc6cccc7c6C(=O)N(C6CCC(=O)NC6=O)C7=O)C5)C4)CC3)cc2cc1NC(=O)c1cccc(C(C)(F)F)n1] |
| I-12 | 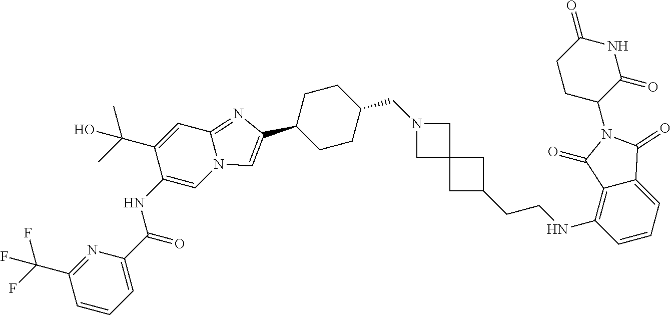 [SMILES: CC(C)(O)c1cc2nc([C@H]3CC[C@H](CN4CC5(CC(CCNc6cccc7c6C(=O)N(C6CCC(=O)NC6=O)C7=O)C5)C4)CC3)cn2cc1NC(=O)c1cccc(C(F)(F)F)n1] |
| I-13 | 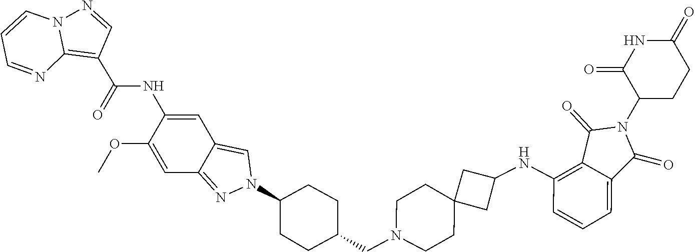 [SMILES: COc1cc2nn([C@H]3CC[C@H](CN4CCC5(CC4)CC(Nc4cccc6c4C(=O)N(C4CCC(=O)NC4=O)C6=O)C5)CC3)cc2cc1NC(=O)c1cnn2cccnc12] |
| I-14 | 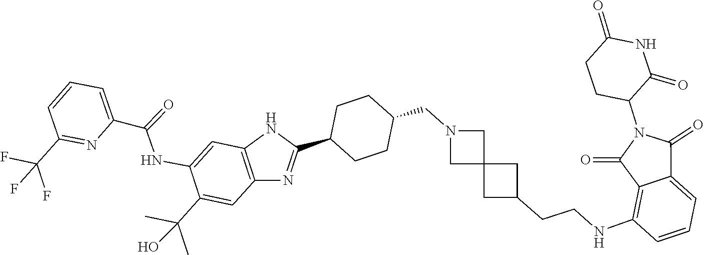 [SMILES: CC(C)(O)c1cc2nc([C@H]3CC[C@H](CN4CC5(CC(CCNc6cccc7c6C(=O)N(C6CCC(=O)NC6=O)C7=O)C5)C4)CC3)[nH]c2cc1NC(=O)c1cccc(C(F)(F)F)n1] |
| I-15 | 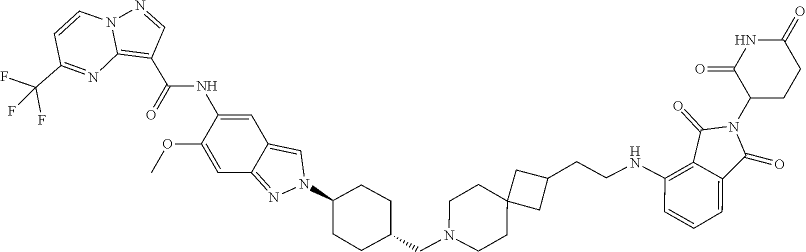 [SMILES: COc1cc2nn([C@H]3CC[C@H](CN4CCC5(CC4)CC(CCNc4cccc6c4C(=O)N(C4CCC(=O)NC4=O)C6=O)C5)CC3)cc2cc1NC(=O)c1cnn2ccc(C(F)(F)F)nc12] |
| I-16 | 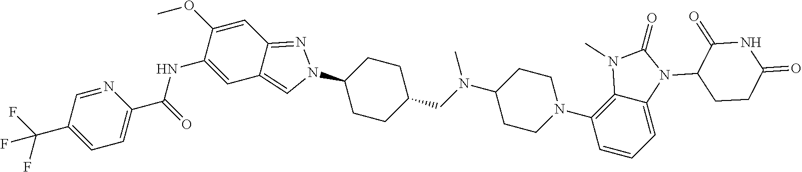 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1ccc(C(F)(F)F)cn1] |
| I-17 | 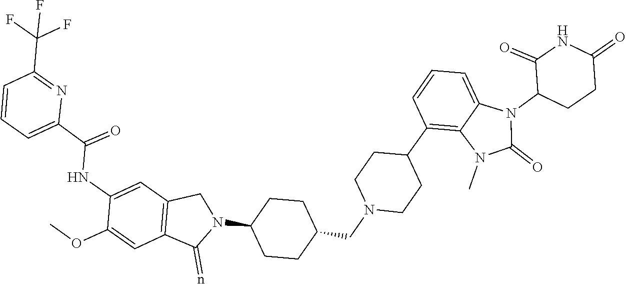 |
| I-18 | 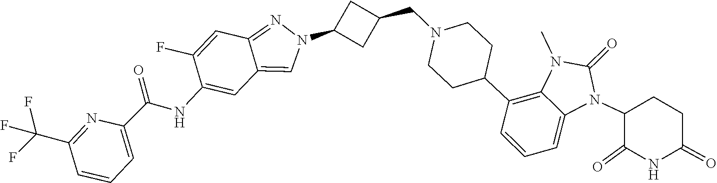 [SMILES: Cn1c(=O)n(C2CCC(=O)NC2=O)c2cccc(C3CCN(C[C@H]4C[C@@H](n5cc6cc(NC(=O)c7cccc(C(F)(F)F)n7)c(F)cc6n5)C4)CC3)c21] |
| I-19 | 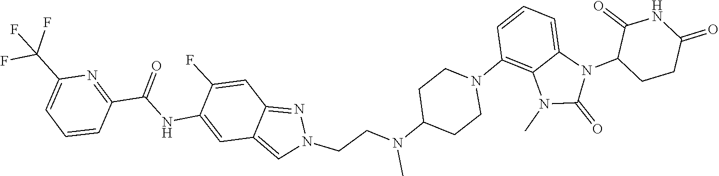 [SMILES: CN(CCn1cc2cc(NC(=O)c3cccc(C(F)(F)F)n3)c(F)cc2n1)C1CCN(c2cccc3c2n(C)c(=O)n3C2CCC(=O)NC2=O)CC1] |
| I-20 | 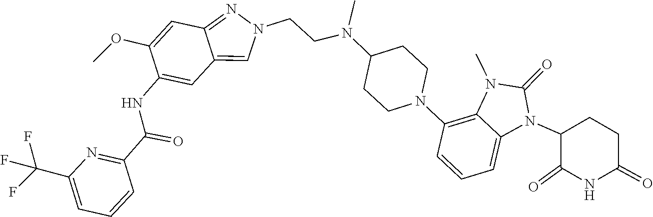 [SMILES: COc1cc2nn(CCN(C)C3CCN(c4cccc5c4n(C)c(=O)n5C4CCC(=O)NC4=O)CC3)cc2cc1NC(=O)c1cccc(C(F)(F)F)n1] |
| I-21 | 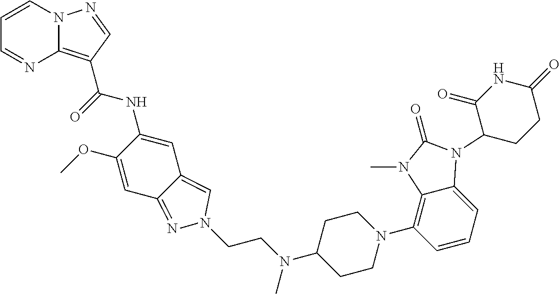 [SMILES: COc1cc2nn(CCN(C)C3CCN(c4cccc5c4n(C)c(=O)n5C4CCC(=O)NC4=O)CC3)cc2cc1NC(=O)c1cnn2cccnc12] |
| I-22 | 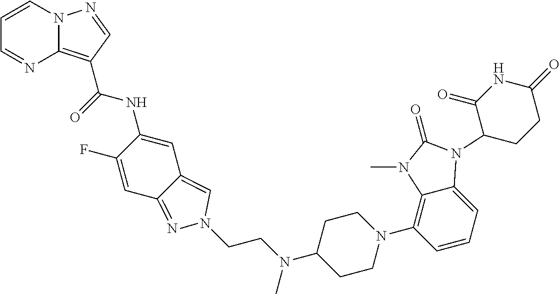 [SMILES: CN(CCn1cc2cc(NC(=O)c3cnn4cccnc34)c(F)cc2n1)C1CCN(c2cccc3c2n(C)c(=O)n3C2CCC(=O)NC2=O)CC1] |
| I-23 | 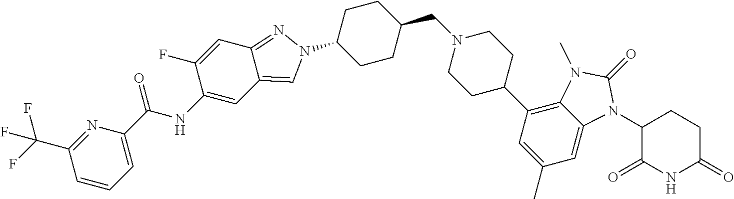 [SMILES: Cc1cc(C2CCN(C[C@H]3CC[C@H](n4cc5cc(NC(=O)c6cccc(C(F)(F)F)n6)c(F)cc5n4)CC3)CC2)c2c(c1)n(C1CCC(=O)NC1=O)c(=O)n2C] |
| I-24 | 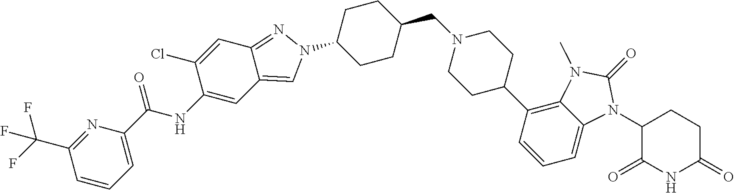 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(Cl)cc6n5)CC4)CC3)c21] |
| I-25 | 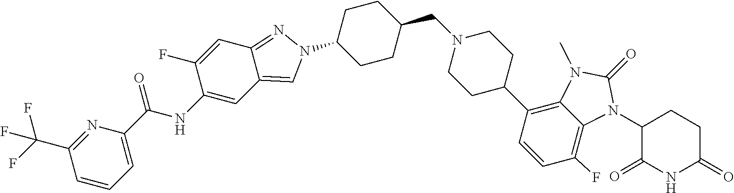 [SMILES: Cn1c(=O)n(C2CCC(=O)NC2=O)c2c(F)ccc(C3CCN(C[C@H]4CC[C@H](n5cc6cc(NC(=O)c7cccc(C(F)(F)F)n7)c(F)cc6n5)CC4)CC3)c21] |
| I-26 | 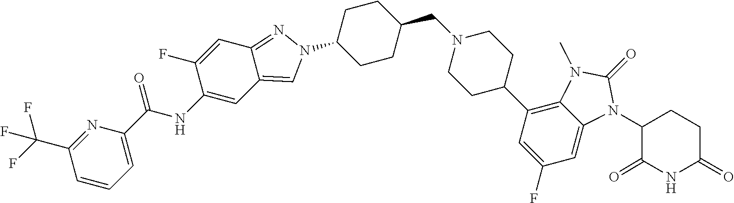 [SMILES: Cn1c(=O)n(C2CCC(=O)NC2=O)c2cc(F)cc(C3CCN(C[C@H]4CC[C@H](n5cc6cc(NC(=O)c7cccc(C(F)(F)F)n7)c(F)cc6n5)CC4)CC3)c21] |
| I-27 | 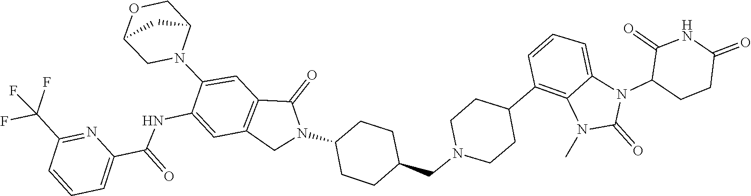 [SMILES: Cn1c(=O)n(C2CCC(=O)NC2=O)c2cccc(C3CCN(C[C@H]4CC[C@H](N5Cc6cc(NC(=O)c7cccc(C(F)(F)F)n7)c(N7C[C@@H]8C[C@H]7CO8)cc6C5=O)CC4)CC3)c21] |
| I-28 | 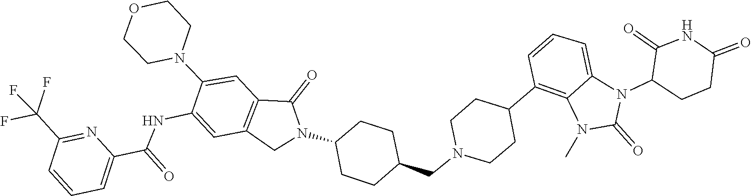 [SMILES: Cn1c(=O)n(C2CCC(=O)NC2=O)c2cccc(C3CCN(C[C@H]4CC[C@H](N5Cc6cc(NC(=O)c7cccc(C(F)(F)F)n7)c(N7CCOCC7)cc6C5=O)CC4)CC3)c21] |
| I-29 | 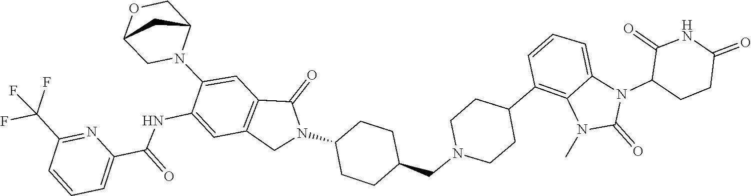 [SMILES: Cn1c(=O)n(C2CCC(=O)NC2=O)c2cccc(C3CCN(C[C@H]4CC[C@H](N5Cc6cc(NC(=O)c7cccc(C(F)(F)F)n7)c(N7C[C@H]8C[C@@H]7CO8)cc6C5=O)CC4)CC3)c21] |
| I-30 | 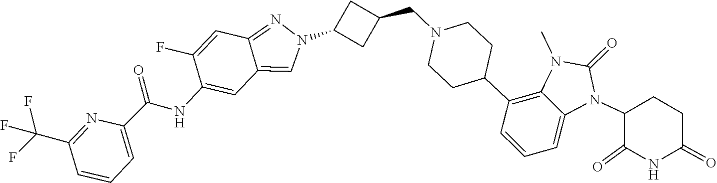 [SMILES: Cn1c(=O)n(C2CCC(=O)NC2=O)c2cccc(C3CCN(C[C@H]4C[C@H](n5cc6cc(NC(=O)c7cccc(C(F)(F)F)n7)c(F)cc6n5)C4)CC3)c21] |
| I-31 | 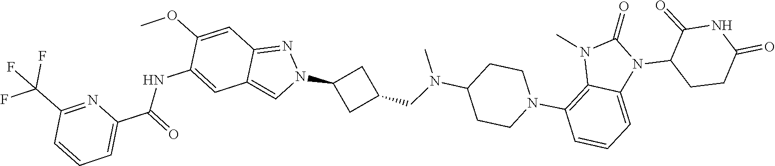 [SMILES: COc1cc2nn([C@H]3C[C@H](CN(C)C4CCN(c5cccc6c5n(C)c(=O)n6C5CCC(=O)NC5=O)CC4)C3)cc2cc1NC(=O)c1cccc(C(F)(F)F)n1] |
| I-32 | 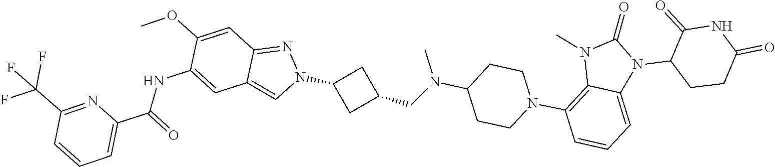 [SMILES: COc1cc2nn([C@H]3C[C@@H](CN(C)C4CCN(c5cccc6c5n(C)c(=O)n6C5CCC(=O)NC5=O)CC4)C3)cc2cc1NC(=O)c1cccc(C(F)(F)F)n1] |
| I-33 | 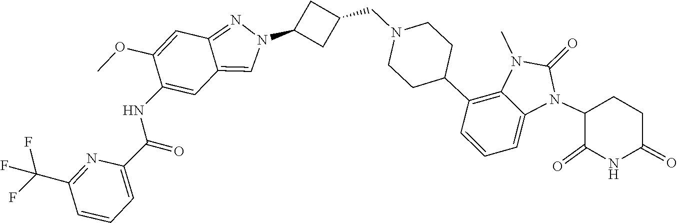 [SMILES: COc1cc2nn([C@H]3C[C@H](CN4CCC(c5cccc6c5n(C)c(=O)n6C5CCC(=O)NC5=O)CC4)C3)cc2cc1NC(=O)c1cccc(C(F)(F)F)n1] |
| I-34 | 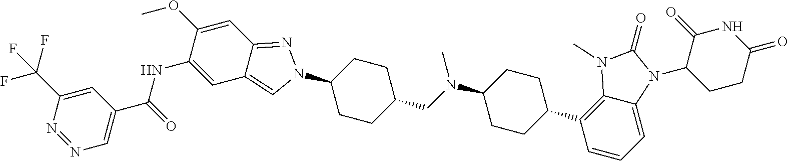 [SMILES: COc1cc2nn([C@H]3CC[C@H](CN(C)[C@H]4CC[C@H](c5cccc6c5n(C)c(=O)n6C5CCC(=O)NC5=O)CC4)CC3)cc2cc1NC(=O)c1cnnc(C(F)(F)F)c1] |
| I-35 | 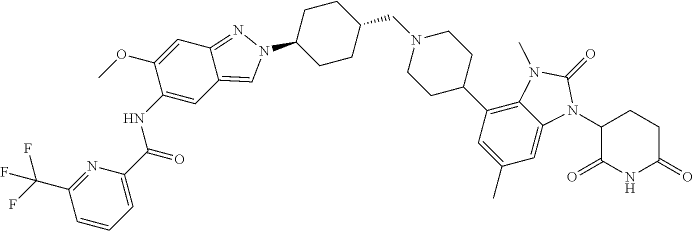 [SMILES: COc1cc2nn([C@H]3CC[C@H](CN4CCC(c5cc(C)cc6c5n(C)c(=O)n6C5CCC(=O)NC5=O)CC4)CC3)cc2cc1NC(=O)c1cccc(C(F)(F)F)n1] |
| I-36 | 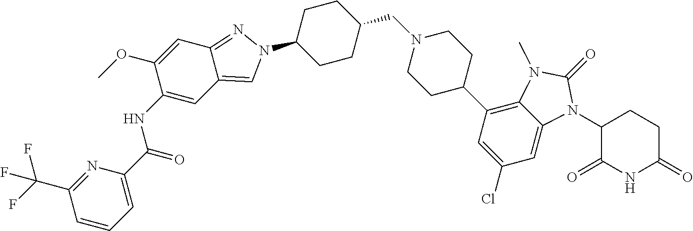 [SMILES: COc1cc2nn([C@H]3CC[C@H](CN4CCC(c5cc(Cl)cc6c5n(C)c(=O)n6C5CCC(=O)NC5=O)CC4)CC3)cc2cc1NC(=O)c1cccc(C(F)(F)F)n1] |
| I-37 | 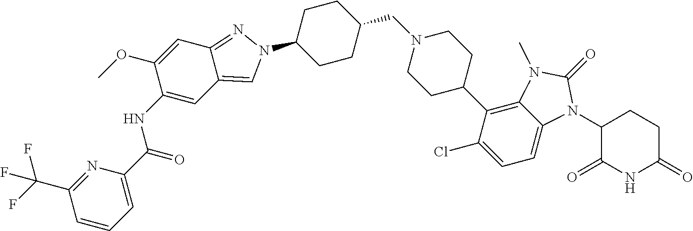 [SMILES: COc1cc2nn([C@H]3CC[C@H](CN4CCC(c5c(Cl)ccc6c5n(C)c(=O)n6C5CCC(=O)NC5=O)CC4)CC3)cc2cc1NC(=O)c1cccc(C(F)(F)F)n1] |
| I-38 | 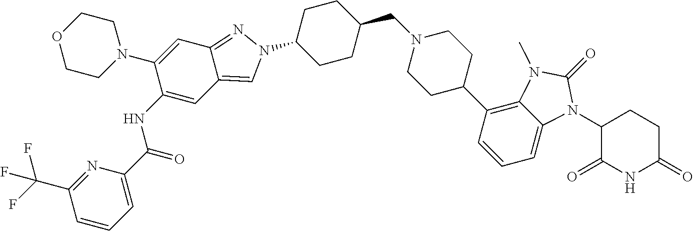 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(N7CCOCC7)cc6n5)CC4)CC3)c21] |
| I-39 | 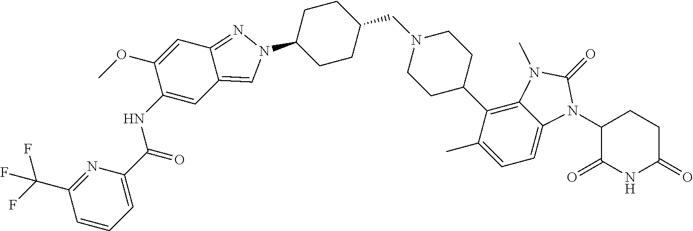 [SMILES: COc1cc2nn([C@H]3CC[C@H](CN4CCC(c5c(C)ccc6c5n(C)c(=O)n6C5CCC(=O)NC5=O)CC4)CC3)cc2cc1NC(=O)c1cccc(C(F)(F)F)n1] |
| I-40 | 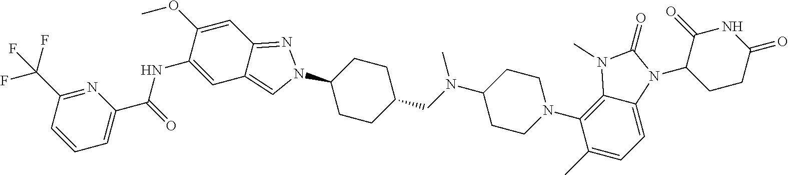 [SMILES: COc1cc2nn([C@H]3CC[C@H](CN(C)C4CCN(c5c(C)ccc6c5n(C)c(=O)n6C5CCC(=O)NC5=O)CC4)CC3)cc2cc1NC(=O)c1cccc(C(F)(F)F)n1] |
| I-41 | 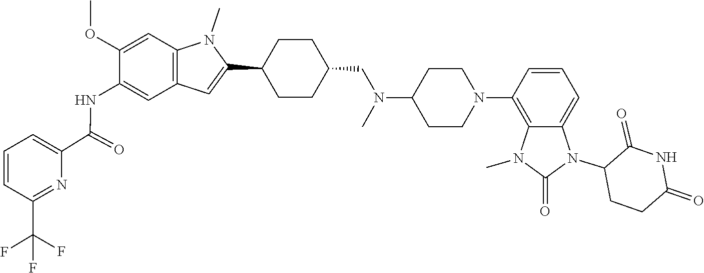 [SMILES: COc1cc2c(cc1NC(=O)c1cccc(C(F)(F)F)n1)cc([C@H]1CC[C@H](CN(C)C3CCN(c4cccc5c4n(C)c(=O)n5C4CCC(=O)NC4=O)CC3)CC1)n2C] |
| I-42 | 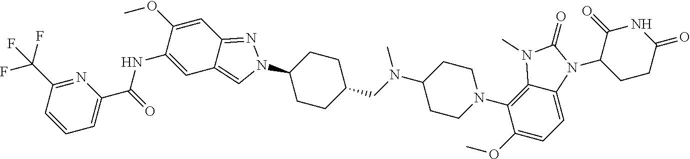 [SMILES: COc1cc2nn([C@H]3CC[C@H](CN(C)C4CCN(c5c(OC)ccc6c5n(C)c(=O)n6C5CCC(=O)NC5=O)CC4)CC3)cc2cc1NC(=O)c1cccc(C(F)(F)F)n1] |
| I-43 | 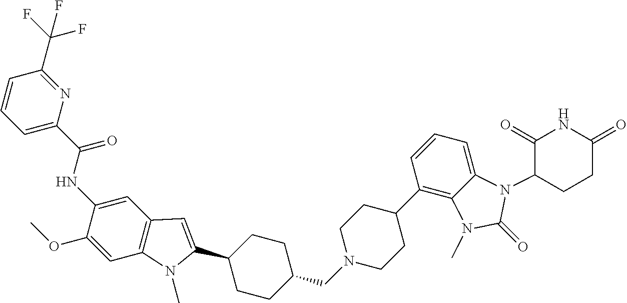 [SMILES: COc1cc2c(cc1NC(=O)c1cccc(C(F)(F)F)n1)cc([C@H]1CC[C@H](CN3CCC(c4cccc5c4n(C)c(=O)n5C4CCC(=O)NC4=O)CC3)CC1)n2C] |
| I-44 | 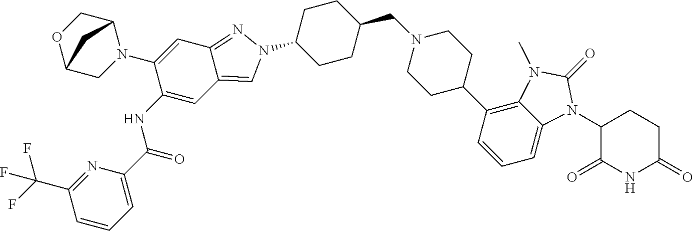 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(N7C[C@H]8C[C@@H]7CO8)cc6n5)CC4)CC3)c21] |
| I-45 | 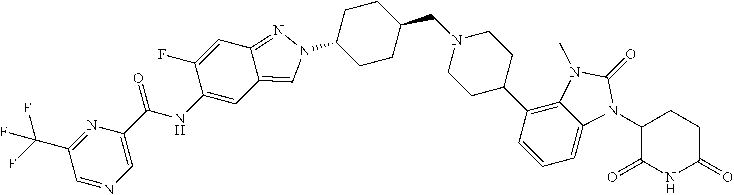 [SMILES: Cn1c(=O)n(C2CCC(=O)NC2=O)c2cccc(C3CCN(C[C@H]4CC[C@H](n5cc6cc(NC(=O)c7cncc(C(F)(F)F)n7)c(F)cc6n5)CC4)CC3)c21] |
| I-46 | 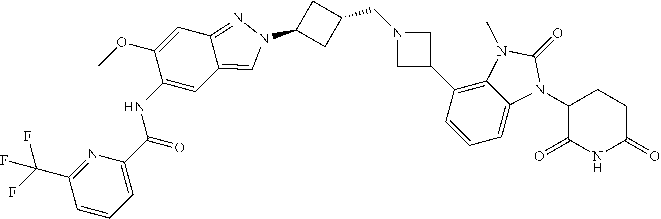 [SMILES: COc1cc2nn([C@H]3C[C@H](CN4CC(c5cccc6c5n(C)c(=O)n6C5CCC(=O)NC5=O)C4)C3)cc2cc1NC(=O)c1cccc(C(F)(F)F)n1] |
| I-47 | 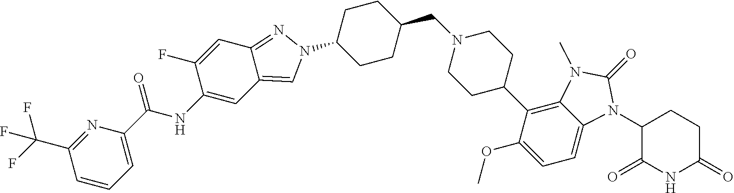 [SMILES: COc1ccc2c(c1C1CCN(C[C@H]3CC[C@H](n4cc5cc(NC(=O)c6cccc(C(F)(F)F)n6)c(F)cc5n4)CC3)CC1)n(C)c(=O)n2C1CCC(=O)NC1=O] |
| I-48 | 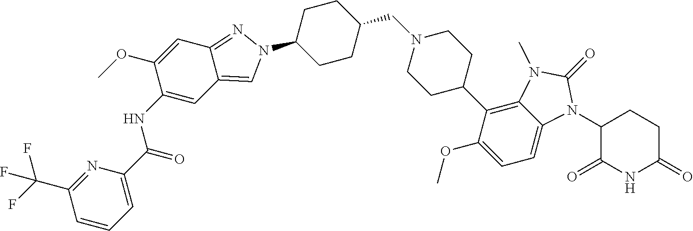 [SMILES: COc1cc2nn([C@H]3CC[C@H](CN4CCC(c5c(OC)ccc6c5n(C)c(=O)n6C5CCC(=O)NC5=O)CC4)CC3)cc2cc1NC(=O)c1cccc(C(F)(F)F)n1] |
| I-49 | 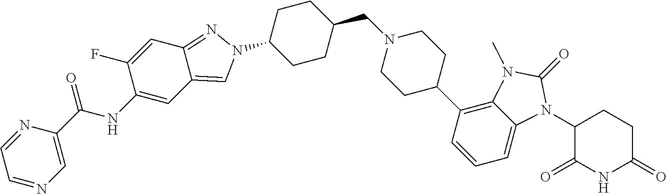 [SMILES: Cn1c(=O)n(C2CCC(=O)NC2=O)c2cccc(C3CCN(C[C@H]4CC[C@H](n5cc6cc(NC(=O)c7cnccn7)c(F)cc6n5)CC4)CC3)c21] |
| I-50 | 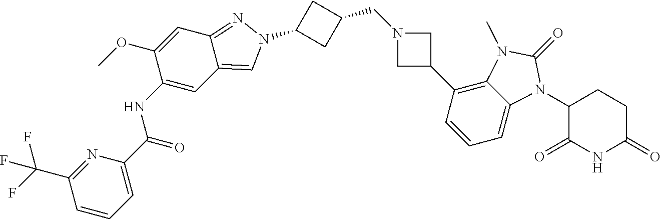 [SMILES: COc1cc2nn([C@H]3C[C@@H](CN4CC(c5cccc6c5n(C)c(=O)n6C5CCC(=O)NC5=O)C4)C3)cc2cc1NC(=O)c1cccc(C(F)(F)F)n1] |
| I-51 | 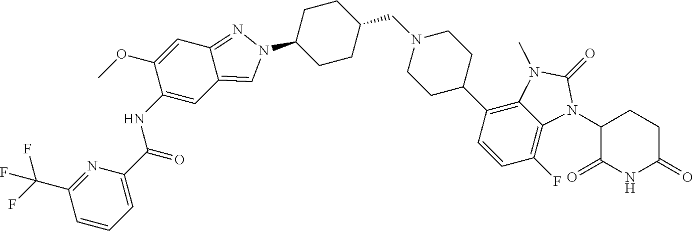 [SMILES: COc1cc2nn([C@H]3CC[C@H](CN4CCC(c5ccc(F)c6c5n(C)c(=O)n6C5CCC(=O)NC5=O)CC4)CC3)cc2cc1NC(=O)c1cccc(C(F)(F)F)n1] |
| I-52 | 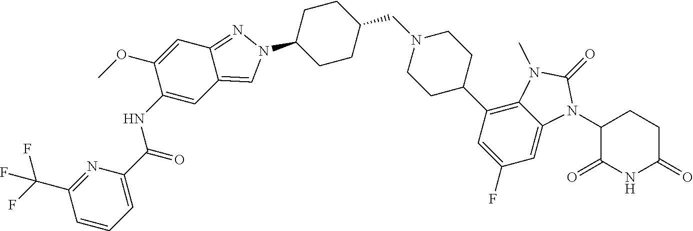 [SMILES: COc1cc2nn([C@H]3CC[C@H](CN4CCC(c5cc(F)cc6c5n(C)c(=O)n6C5CCC(=O)NC5=O)CC4)CC3)cc2cc1NC(=O)c1cccc(C(F)(F)F)n1] |
| I-53 | 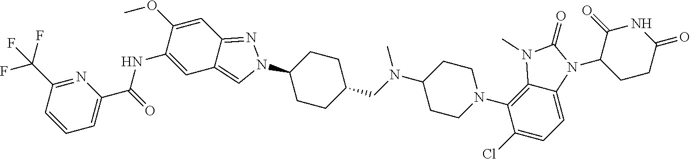 [SMILES: COc1cc2nn([C@H]3CC[C@H](CN(C)C4CCN(c5c(Cl)ccc6c5n(C)c(=O)n6C5CCC(=O)NC5=O)CC4)CC3)cc2cc1NC(=O)c1cccc(C(F)(F)F)n1] |
| I-54 | 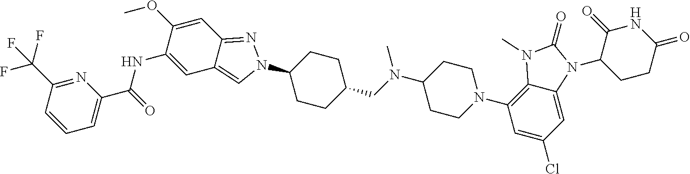 [SMILES: COc1cc2nn([C@H]3CC[C@H](CN(C)C4CCN(c5cc(Cl)cc6c5n(C)c(=O)n6C5CCC(=O)NC5=O)CC4)CC3)cc2cc1NC(=O)c1cccc(C(F)(F)F)n1] |
| I-55 | 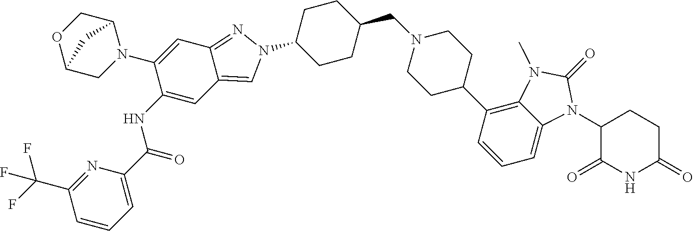 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(N7C[C@@H]8C[C@H]7CO8)cc6n5)CC4)CC3)c21] |
| I-56 | 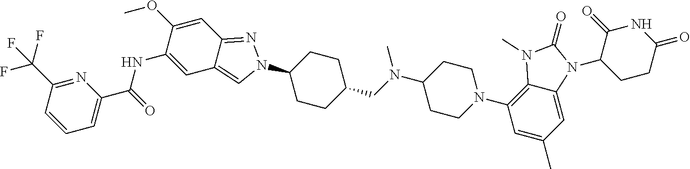 [SMILES: COc1cc2nn([C@H]3CC[C@H](CN(C)C4CCN(c5cc(C)cc6c5n(C)c(=O)n6C5CCC(=O)NC5=O)CC4)CC3)cc2cc1NC(=O)c1cccc(C(F)(F)F)n1] |
| I-57 | 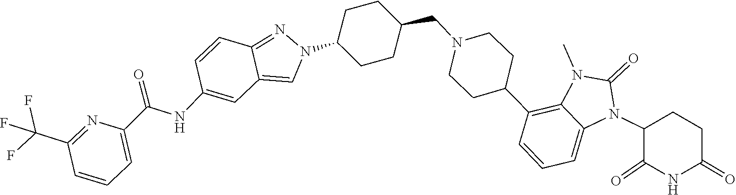 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)ccc6n5)CC4)CC3)c21] |
| I-58 | 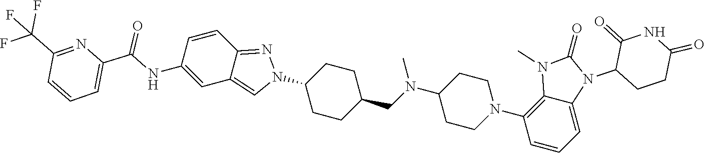 [SMILES: CN(C[C@H]1CC[C@H](n2cc3cc(NC(=O)c4cccc(C(F)(F)F)n4)ccc3n2)CC1)C1CCN(c2cccc3c2n(C)c(=O)n3C2CCC(=O)NC2=O)CC1] |
| I-59 | 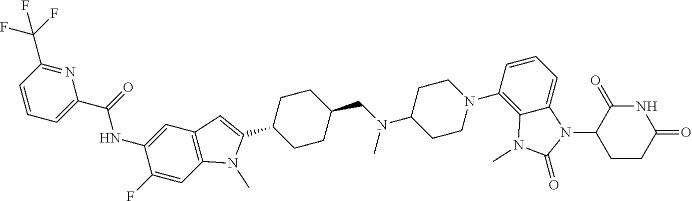 [SMILES: CN(C[C@H]1CC[C@H](c2cc3cc(NC(=O)c4cccc(C(F)(F)F)n4)c(F)cc3n2C)CC1)C1CCN(c2cccc3c2n(C)c(=O)n3C2CCC(=O)NC2=O)CC1] |
| I-60 | 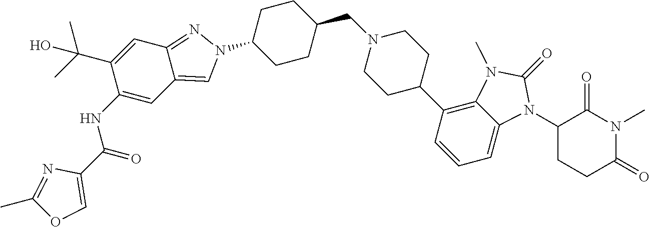 |
| I-61 | 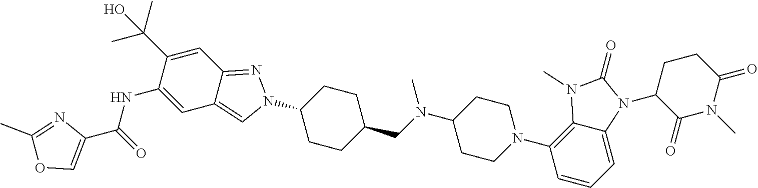 [SMILES: Cc1nc(C(=O)Nc2cc3cn([C@H]4CC[C@H](CN(C)C5CCN(c6cccc7c6n(C)c(=O)n7C6CCC(=O)N(C)C6=O)CC5)CC4)nc3cc2C(C)(C)O)co1] |
| I-62 | 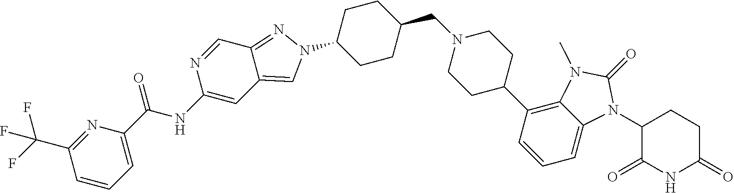 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)ncc6n5)CC4)CC3)c21] |
| I-63 | 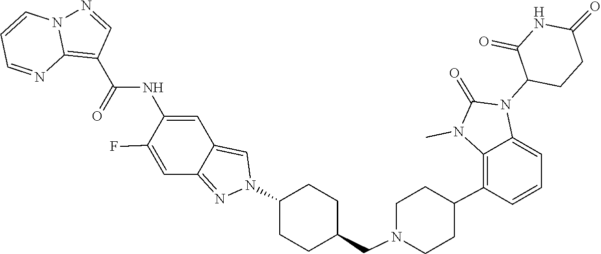 [SMILES: Cn1c(=O)n(C2CCC(=O)NC2=O)c2cccc(C3CCN(C[C@H]4CC[C@H](n5cc6cc(NC(=O)c7cnn8cccnc78)c(F)cc6n5)CC4)CC3)c21] |
| I-64 | 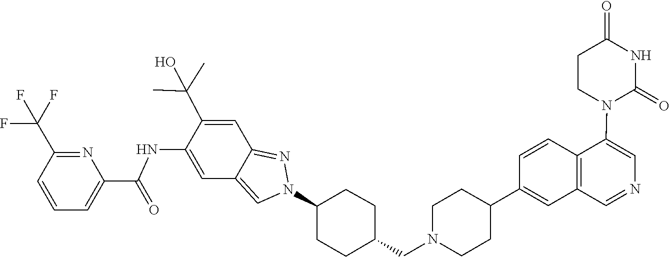 [SMILES: CC(C)(O)c1cc2nn([C@H]3CC[C@H](CN4CCC(c5ccc6c(N7CCC(=O)NC7=O)cncc6c5)CC4)CC3)cc2cc1NC(=O)c1cccc(C(F)(F)F)n1] |
| I-65 | 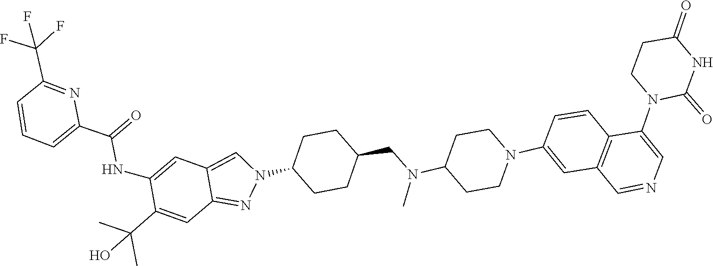 [SMILES: CN(C[C@H]1CC[C@H](n2cc3cc(NC(=O)c4cccc(C(F)(F)F)n4)c(C(C)(C)O)cc3n2)CC1)C1CCN(c2ccc3c(N4CCC(=O)NC4=O)cncc3c2)CC1] |
| I-66 | 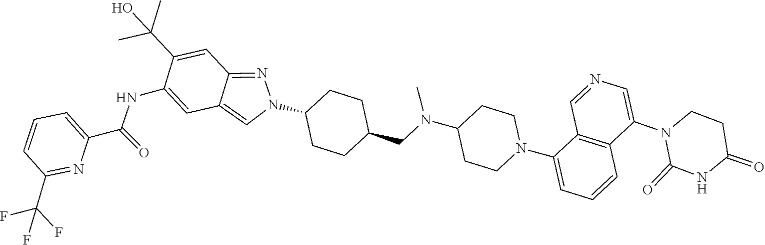 [SMILES: CN(C[C@H]1CC[C@H](n2cc3cc(NC(=O)c4cccc(C(F)(F)F)n4)c(C(C)(C)O)cc3n2)CC1)C1CCN(c2cccc3c(N4CCC(=O)NC4=O)cncc23)CC1] |
| I-67 | 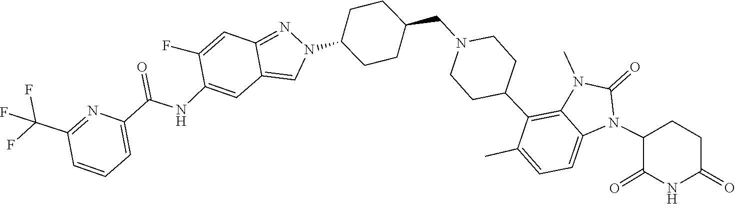 [SMILES: Cc1ccc2c(c1C1CCN(C[C@H]3CC[C@H](n4cc5cc(NC(=O)c6cccc(C(F)(F)F)n6)c(F)cc5n4)CC3)CC1)n(C)c(=O)n2C1CCC(=O)NC1=O] |
| I-68 | 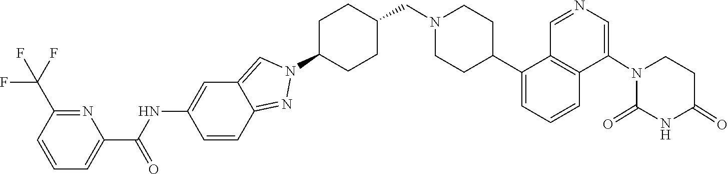 [SMILES: O=C1CCN(c2cncc3c(C4CCN(C[C@H]5CC[C@H](n6cc7cc(NC(=O)c8cccc(C(F)(F)F)n8)ccc7n6)CC5)CC4)cccc23)C(=O)N1] |
| I-69 | 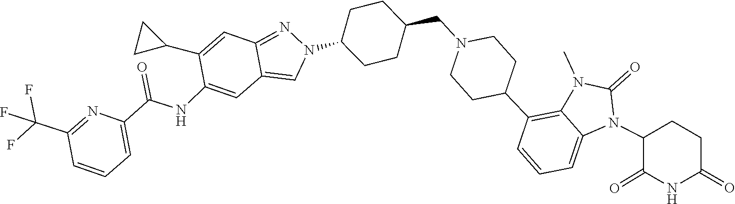 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(C7CC7)cc6n5)CC4)CC3)c21] |
| I-70 | 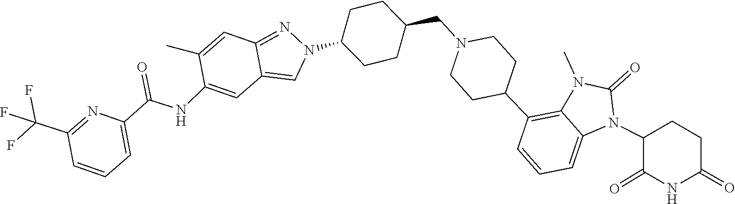 [SMILES: Cc1cc2nn([C@H]3CC[C@H](CN4CCC(c5cccc6c5n(C)c(=O)n6C5CCC(=O)NC5=O)CC4)CC3)cc2cc1NC(=O)c1cccc(C(F)(F)F)n1] |
| I-71 | 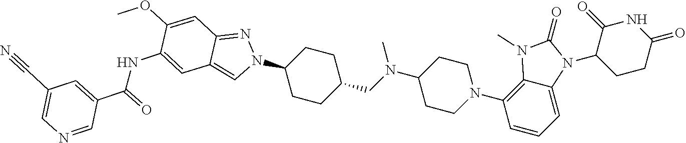 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1cncc(C#N)c1] |
| I-72 | 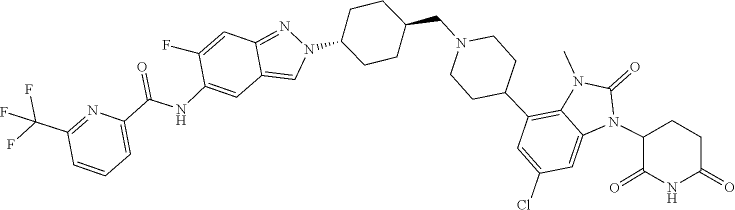 [SMILES: Cn1c(=O)n(C2CCC(=O)NC2=O)c2cc(Cl)cc(C3CCN(C[C@H]4CC[C@H](n5cc6cc(NC(=O)c7cccc(C(F)(F)F)n7)c(F)cc6n5)CC4)CC3)c21] |
| I-73 | 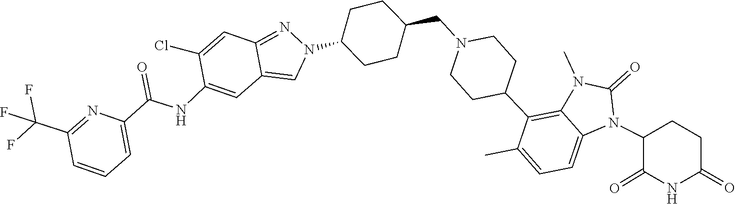 [SMILES: Cc1ccc2c(c1C1CCN(C[C@H]3CC[C@H](n4cc5cc(NC(=O)c6cccc(C(F)(F)F)n6)c(Cl)cc5n4)CC3)CC1)n(C)c(=O)n2C1CCC(=O)NC1=O] |
| I-74 | 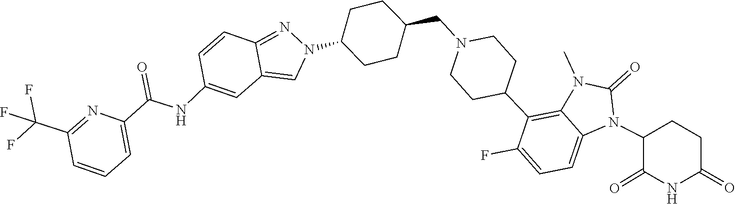 [SMILES: Cn1c(=O)n(C2CCC(=O)NC2=O)c2ccc(F)c(C3CCN(C[C@H]4CC[C@H](n5cc6cc(NC(=O)c7cccc(C(F)(F)F)n7)ccc6n5)CC4)CC3)c21] |
| I-75 | 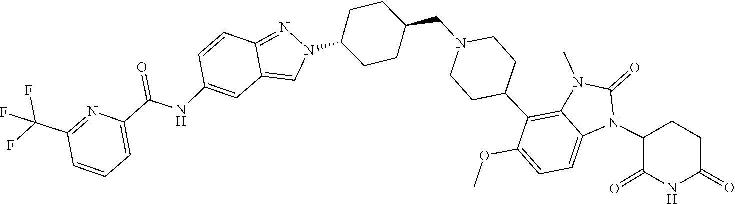 [SMILES: COc1ccc2c(c1C1CCN(C[C@H]3CC[C@H](n4cc5cc(NC(=O)c6cccc(C(F)(F)F)n6)ccc5n4)CC3)CC1)n(C)c(=O)n2C1CCC(=O)NC1=O] |
| I-76 | 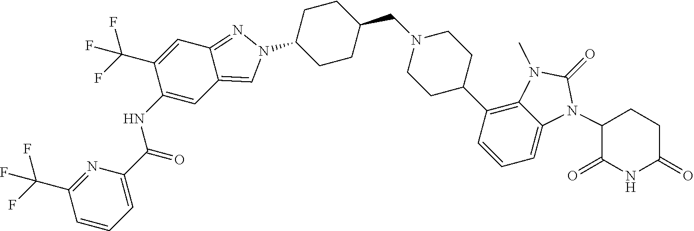 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(C(F)(F)F)cc6n5)CC4)CC3)c21] |
| I-77 | 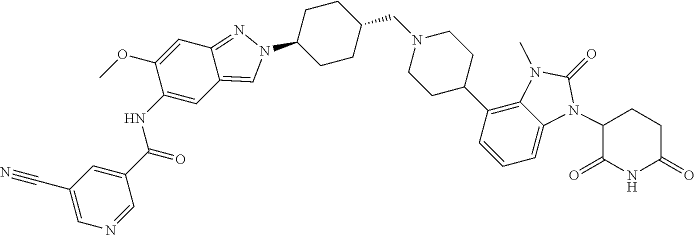 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cncc(C#N)c1] |
| I-78 | 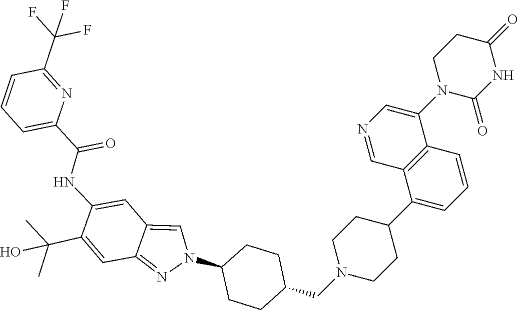 [SMILES: CC(C)(O)c1cc2nn([C@H]3CC[C@H](CN4CCC(c5cccc6c(N7CCC(=O)NC7=O)cncc56)CC4)CC3)cc2cc1NC(=O)c1cccc(C(F)(F)F)n1] |
| I-79 | 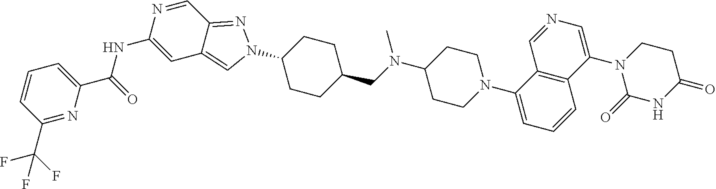 [SMILES: CN(C[C@H]1CC[C@H](n2cc3cc(NC(=O)c4cccc(C(F)(F)F)n4)ncc3n2)CC1)C1CCN(c2cccc3c(N4CCC(=O)NC4=O)cncc23)CC1] |
| I-80 | 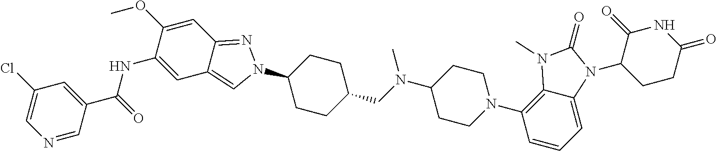 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1cncc(Cl)c1] |
| I-81 | 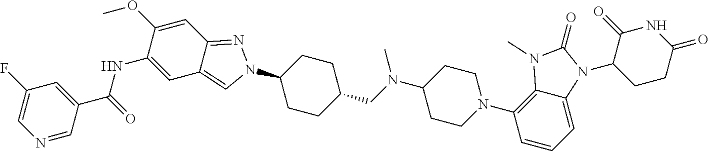 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1cncc(F)c1] |
| I-82 | 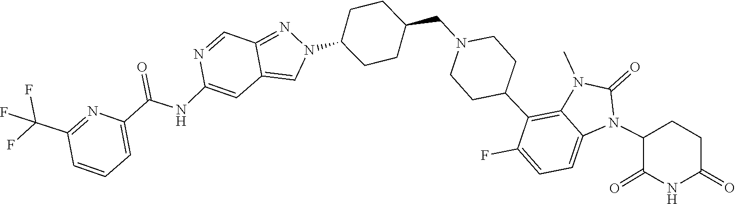 [SMILES: Cn1c(=O)n(C2CCC(=O)NC2=O)c2ccc(F)c(C3CCN(C[C@H]4CC[C@H](n5cc6cc(NC(=O)c7cccc(C(F)(F)F)n7)ncc6n5)CC4)CC3)c21] |
| I-83 | 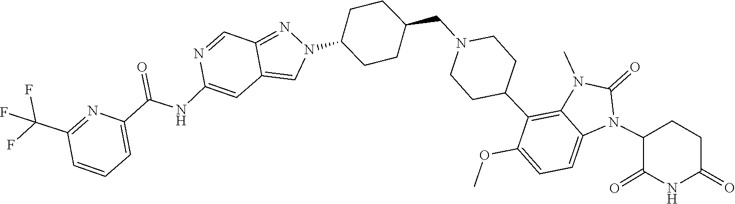 [SMILES: COc1ccc2c(c1C1CCN(C[C@H]3CC[C@H](n4cc5cc(NC(=O)c6cccc(C(F)(F)F)n6)ncc5n4)CC3)CC1)n(C)c(=O)n2C1CCC(=O)NC1=O] |
| I-84 | 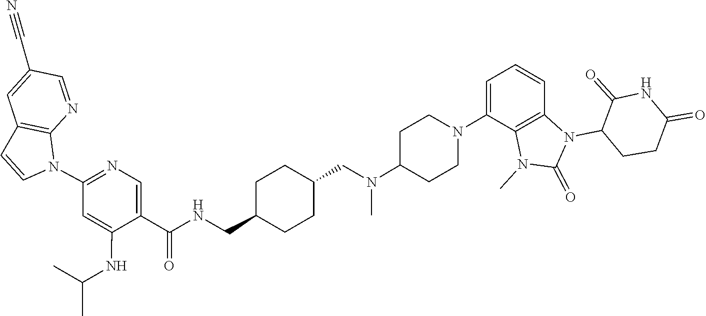 [SMILES: CC(C)Nc1cc(-n2ccc3cc(C#N)cnc32)ncc1C(=O)NC[C@H]1CC[C@H](CN(C)C2CCN(c3cccc4c3n(C)c(=O)n4C3CCC(=O)NC3=O)CC2)CC1] |
| I-85 | 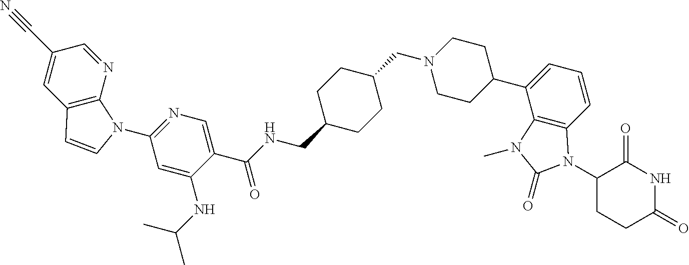 [SMILES: CC(C)Nc1cc(-n2ccc3cc(C#N)cnc32)ncc1C(=O)NC[C@H]1CC[C@H](CN2CCC(c3cccc4c3n(C)c(=O)n4C3CCC(=O)NC3=O)CC2)CC1] |
| I-86 | 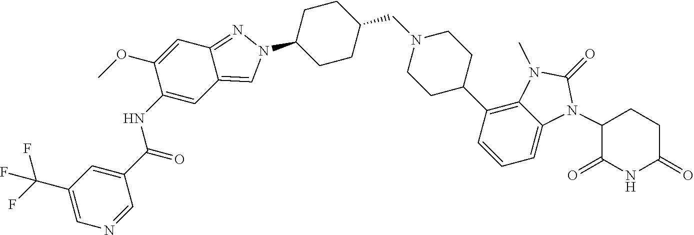 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cncc(C(F)(F)F)c1] |
| I-87 | 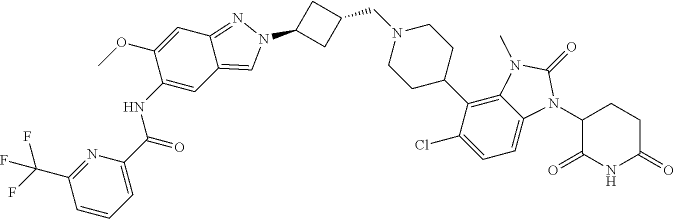 [SMILES: COc1cc2nn([C@H]3C[C@H](CN4CCC(c5c(Cl)ccc6c5n(C)c(=O)n6C5CCC(=O)NC5=O)CC4)C3)cc2cc1NC(=O)c1cccc(C(F)(F)F)n1] |
| I-88 | 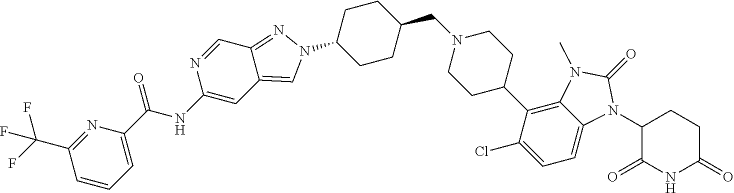 [SMILES: Cn1c(=O)n(C2CCC(=O)NC2=O)c2ccc(Cl)c(C3CCN(C[C@H]4CC[C@H](n5cc6cc(NC(=O)c7cccc(C(F)(F)F)n7)ncc6n5)CC4)CC3)c21] |
| I-89 | 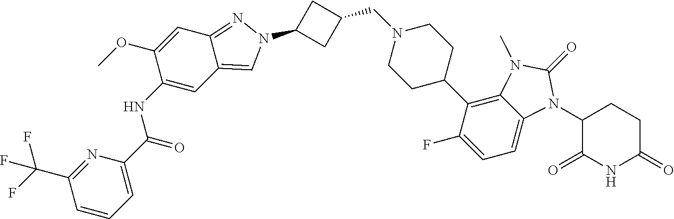 [SMILES: COc1cc2nn([C@H]3C[C@H](CN4CCC(c5c(F)ccc6c5n(C)c(=O)n6C5CCC(=O)NC5=O)CC4)C3)cc2cc1NC(=O)c1cccc(C(F)(F)F)n1] |
| I-90 | 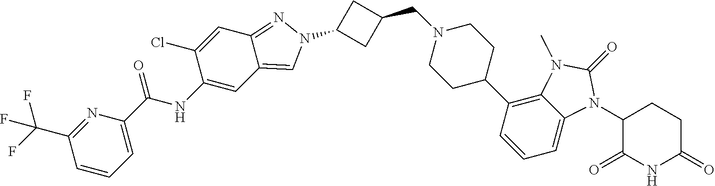 [SMILES: Cn1c(=O)n(C2CCC(=O)NC2=O)c2cccc(C3CCN(C[C@H]4C[C@H](n5cc6cc(NC(=O)c7cccc(C(F)(F)F)n7)c(Cl)cc6n5)C4)CC3)c21] |
| I-91 | 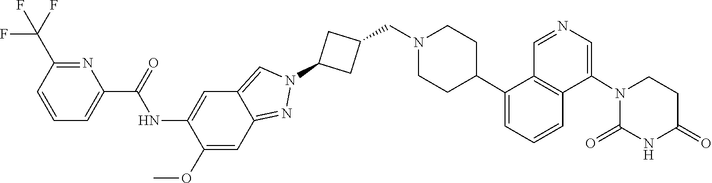 [SMILES: COc1cc2nn([C@H]3C[C@H](CN4CCC(c5cccc6c(N7CCC(=O)NC7=O)cncc56)CC4)C3)cc2cc1NC(=O)c1cccc(C(F)(F)F)n1] |
| I-92 | 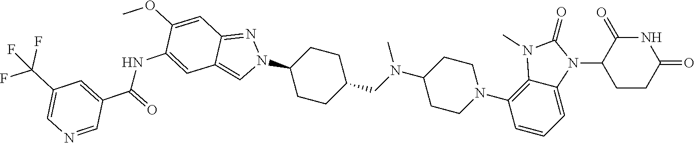 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1cncc(C(F)(F)F)c1] |
| I-93 | 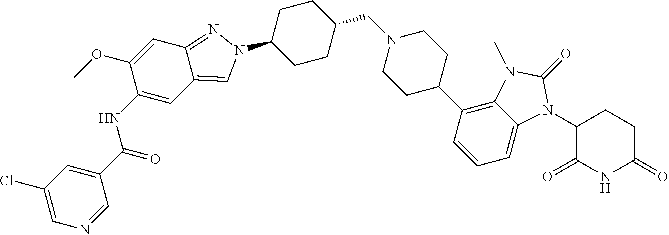 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cncc(Cl)c1] |
| I-94 | 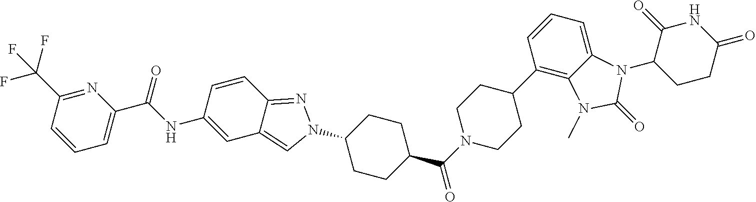 [SMILES: Cn1c(=O)n(C2CCC(=O)NC2=O)c2cccc(C3CCN(C(=O)[C@H]4CC[C@H](n5cc6cc(NC(=O)c7cccc(C(F)(F)F)n7)ccc6n5)CC4)CC3)c21] |
| I-95 | 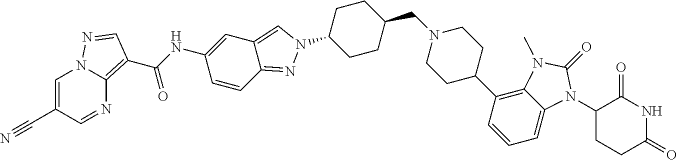 [SMILES: Cn1c(=O)n(C2CCC(=O)NC2=O)c2cccc(C3CCN(C[C@H]4CC[C@H](n5cc6cc(NC(=O)c7cnn8cc(C#N)cnc78)ccc6n5)CC4)CC3)c21] |
| I-96 | 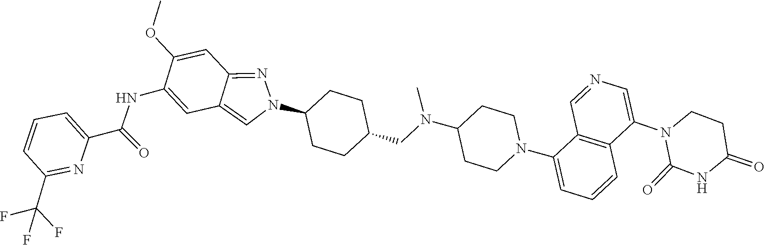 [SMILES: COc1cc2nn([C@H]3CC[C@H](CN(C)C4CCN(c5cccc6c(N7CCC(=O)NC7=O)cncc56)CC4)CC3)cc2cc1NC(=O)c1cccc(C(F)(F)F)n1] |
| I-97 | 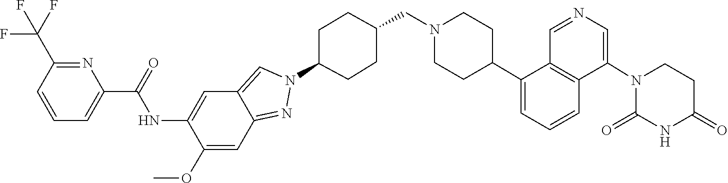 [SMILES: COc1cc2nn([C@H]3CC[C@H](CN4CCC(c5cccc6c(N7CCC(=O)NC7=O)cncc56)CC4)CC3)cc2cc1NC(=O)c1cccc(C(F)(F)F)n1] |
| I-98 | 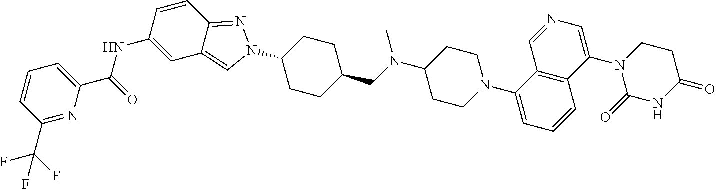 [SMILES: CN(C[C@H]1CC[C@H](n2cc3cc(NC(=O)c4cccc(C(F)(F)F)n4)ccc3n2)CC1)C1CCN(c2cccc3c(N4CCC(=O)NC4=O)cncc23)CC1] |
| I-99 | 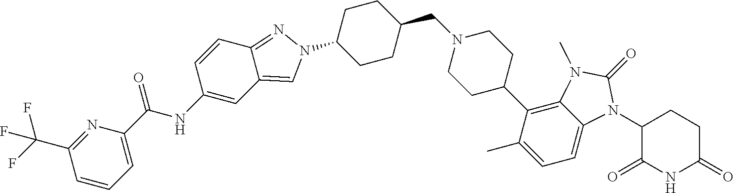 [SMILES: Cc1ccc2c(c1C1CCN(C[C@H]3CC[C@H](n4cc5cc(NC(=O)c6cccc(C(F)(F)F)n6)ccc5n4)CC3)CC1)n(C)c(=O)n2C1CCC(=O)NC1=O] |
| I-100 | 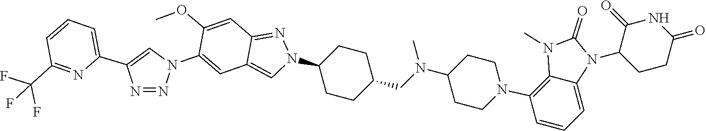 [SMILES: COc1cc2nn([C@H]3CC[C@H](CN(C)C4CCN(c5cccc6c5n(C)c(=O)n6C5CCC(=O)NC5=O)CC4)CC3)cc2cc1-n1cc(-c2cccc(C(F)(F)F)n2)nn1] |
| I-101 | 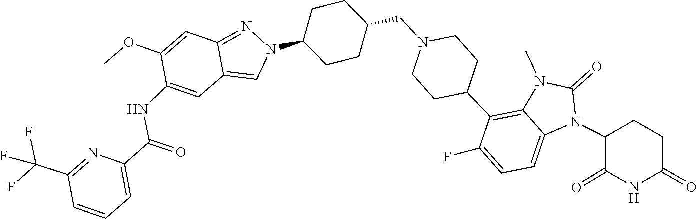 [SMILES: COc1cc2nn([C@H]3CC[C@H](CN4CCC(c5c(F)ccc6c5n(C)c(=O)n6C5CCC(=O)NC5=O)CC4)CC3)cc2cc1NC(=O)c1cccc(C(F)(F)F)n1] |
| I-102 | 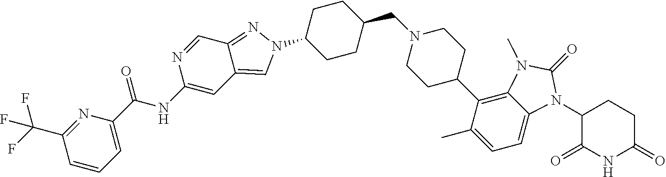 [SMILES: Cc1ccc2c(c1C1CCN(C[C@H]3CC[C@H](n4cc5cc(NC(=O)c6cccc(C(F)(F)F)n6)ncc5n4)CC3)CC1)n(C)c(=O)n2C1CCC(=O)NC1=O] |
| I-103 | 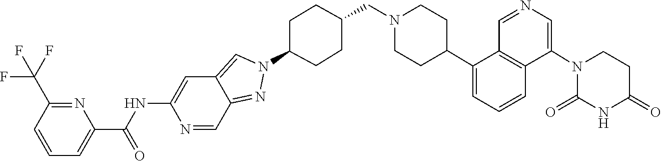 [SMILES: O=C1CCN(c2cncc3c(C4CCN(C[C@H]5CC[C@H](n6cc7cc(NC(=O)c8cccc(C(F)(F)F)n8)ncc7n6)CC5)CC4)cccc23)C(=O)N1] |
| I-104 | 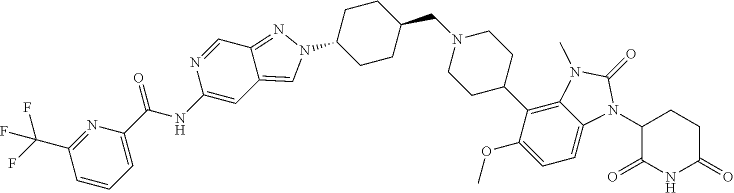 [SMILES: COc1ccc2c(c1C1CCN(C[C@H]3CC[C@H](n4cc5cc(NC(=O)c6cccc(C(F)(F)F)n6)ncc5n4)CC3)CC1)n(C)c(=O)n2C1CCC(=O)NC1=O] |
| I-105 | 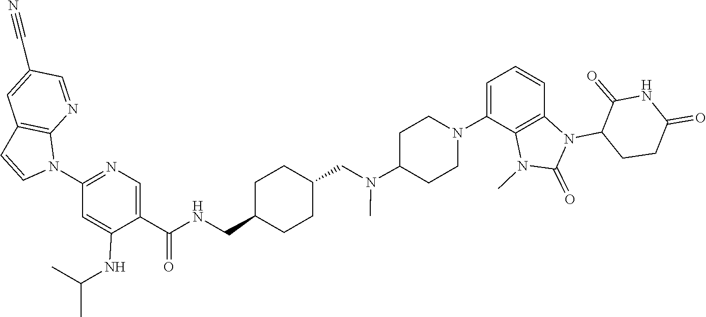 [SMILES: CC(C)Nc1cc(-n2ccc3cc(C#N)cnc32)ncc1C(=O)NC[C@H]1CC[C@H](CN(C)C2CCN(c3cccc4c3n(C)c(=O)n4C3CCC(=O)NC3=O)CC2)CC1] |
| I-106 | 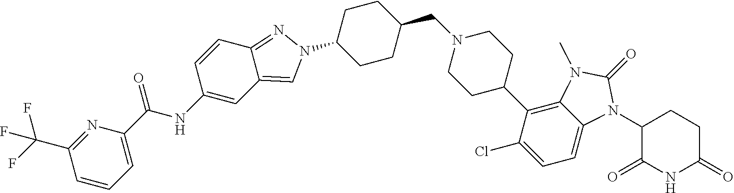 [SMILES: Cn1c(=O)n(C2CCC(=O)NC2=O)c2ccc(Cl)c(C3CCN(C[C@H]4CC[C@H](n5cc6cc(NC(=O)c7cccc(C(F)(F)F)n7)ccc6n5)CC4)CC3)c21] |
| I-107 | 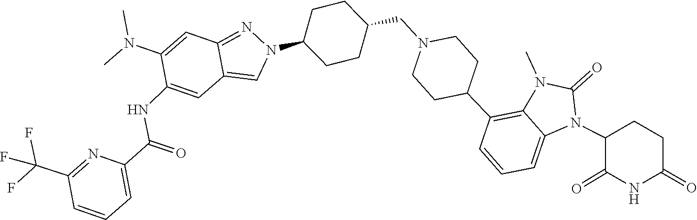 [SMILES: CN(C)c1cc2nn([C@H]3CC[C@H](CN4CCC(c5cccc6c5n(C)c(=O)n6C5CCC(=O)NC5=O)CC4)CC3)cc2cc1NC(=O)c1cccc(C(F)(F)F)n1] |
| I-108 | 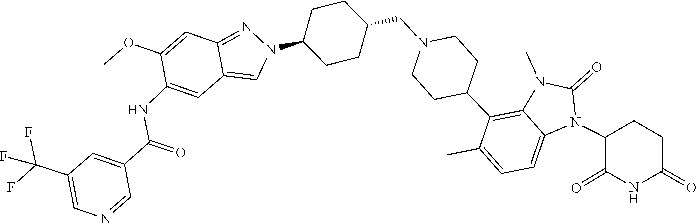 [SMILES: COc1cc2nn([C@H]3CC[C@H](CN4CCC(c5c(C)ccc6c5n(C)c(=O)n6C5CCC(=O)NC5=O)CC4)CC3)cc2cc1NC(=O)c1cncc(C(F)(F)F)c1] |
| I-109 | 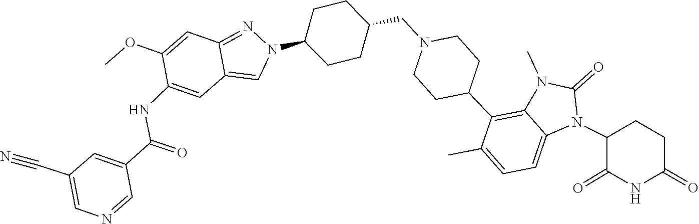 [SMILES: COc1cc2nn([C@H]3CC[C@H](CN4CCC(c5c(C)ccc6c5n(C)c(=O)n6C5CCC(=O)NC5=O)CC4)CC3)cc2cc1NC(=O)c1cncc(C#N)c1] |
| I-110 | 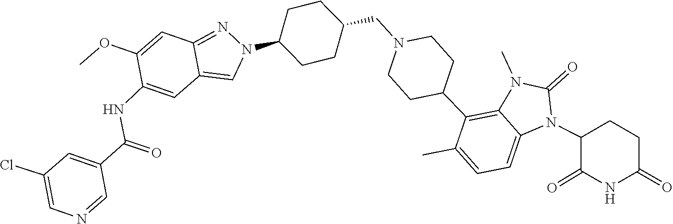 [SMILES: COc1cc2nn([C@H]3CC[C@H](CN4CCC(c5c(C)ccc6c5n(C)c(=O)n6C5CCC(=O)NC5=O)CC4)CC3)cc2cc1NC(=O)c1cncc(Cl)c1] |
| I-111 | 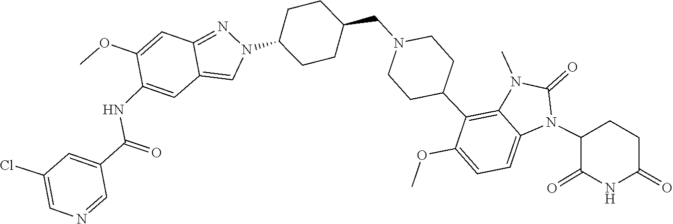 [SMILES: COc1cc2nn([C@H]3CC[C@H](CN4CCC(c5c(OC)ccc6c5n(C)c(=O)n6C5CCC(=O)NC5=O)CC4)CC3)cc2cc1NC(=O)c1cncc(Cl)c1] |
| I-112 | 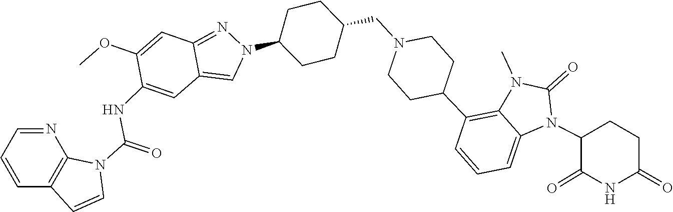 [SMILES: COc1cc2nn([C@H]3CC[C@H](CN4CCC(c5cccc6c5n(C)c(=O)n6C5CCC(=O)NC5=O)CC4)CC3)cc2cc1NC(=O)n1ccc2cccnc21] |
| I-113 | 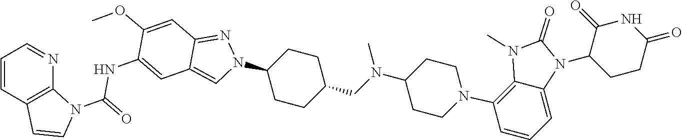 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)n1ccc2cccnc21] |
| I-114 | 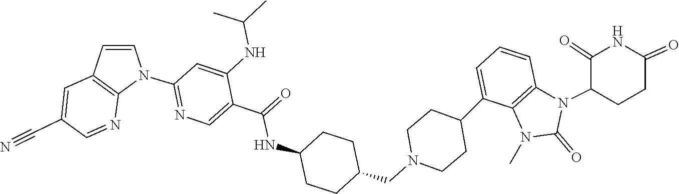 [SMILES: CC(C)Nc1cc(-n2ccc3cc(C#N)cnc32)ncc1C(=O)N[C@H]1CC[C@H](CN2CCC(c3cccc4c3n(C)c(=O)n4C3CCC(=O)NC3=O)CC2)CC1] |
| I-115 | 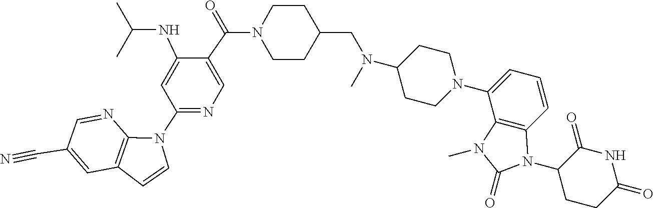 [SMILES: CC(C)Nc1cc(-n2ccc3cc(C#N)cnc32)ncc1C(=O)N1CCC(CN(C)C2CCN(c3cccc4c3n(C)c(=O)n4C3CCC(=O)NC3=O)CC2)CC1] |
| I-116 | 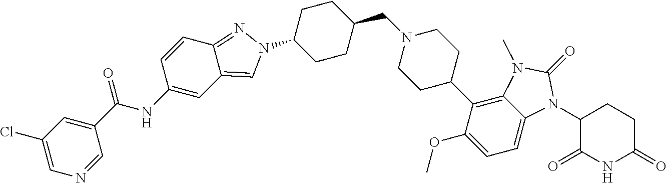 [SMILES: COc1ccc2c(c1C1CCN(C[C@H]3CC[C@H](n4cc5cc(NC(=O)c6cncc(Cl)c6)ccc5n4)CC3)CC1)n(C)c(=O)n2C1CCC(=O)NC1=O] |
| I-117 | 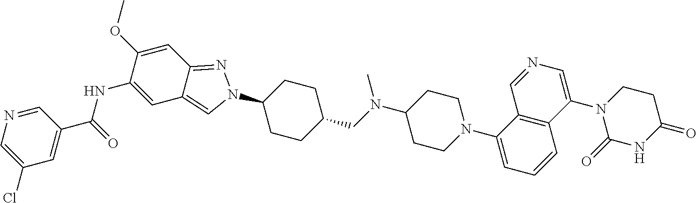 [SMILES: COc1cc2nn([C@H]3CC[C@H](CN(C)C4CCN(c5cccc6c(N7CCC(=O)NC7=O)cncc56)CC4)CC3)cc2cc1NC(=O)c1cncc(Cl)c1] |
| I-118 | 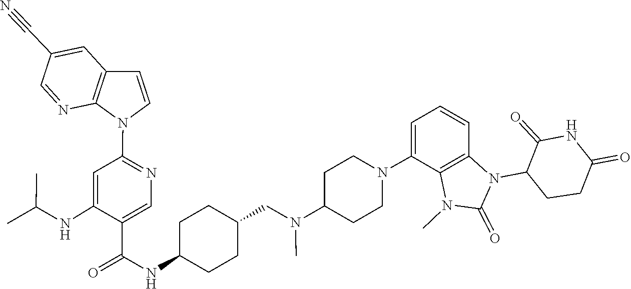 [SMILES: CC(C)Nc1cc(-n2ccc3cc(C#N)cnc32)ncc1C(=O)N[C@H]1CC[C@H](CN(C)C2CCN(c3cccc4c3n(C)c(=O)n4C3CCC(=O)NC3=O)CC2)CC1] |
| I-119 | 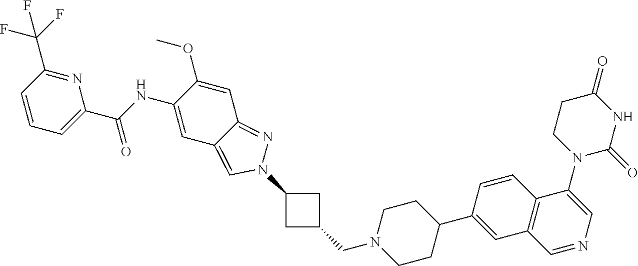 [SMILES: COc1cc2nn([C@H]3C[C@H](CN4CCC(c5ccc6c(N7CCC(=O)NC7=O)cncc6c5)CC4)C3)cc2cc1NC(=O)c1cccc(C(F)(F)F)n1] |
| I-120 | 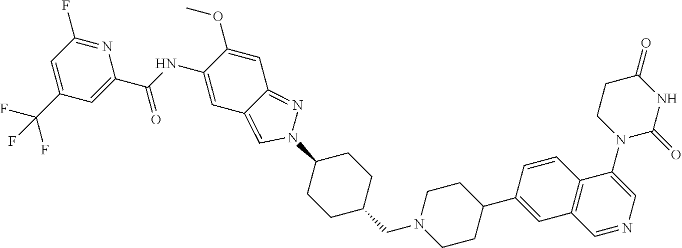 [SMILES: COc1cc2nn([C@H]3CC[C@H](CN4CCC(c5ccc6c(N7CCC(=O)NC7=O)cncc6c5)CC4)CC3)cc2cc1NC(=O)c1cc(C(F)(F)F)cc(F)n1] |
| I-121 | 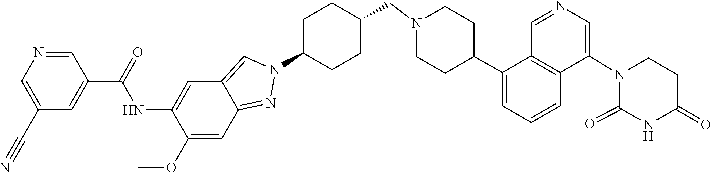 [SMILES: COc1cc2nn([C@H]3CC[C@H](CN4CCC(c5cccc6c(N7CCC(=O)NC7=O)cncc56)CC4)CC3)cc2cc1NC(=O)c1cncc(C#N)c1] |
| I-122 | 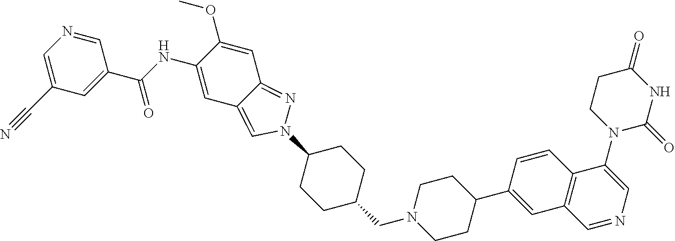 [SMILES: COc1cc2nn([C@H]3CC[C@H](CN4CCC(c5ccc6c(N7CCC(=O)NC7=O)cncc6c5)CC4)CC3)cc2cc1NC(=O)c1cncc(C#N)c1] |
| I-123 | 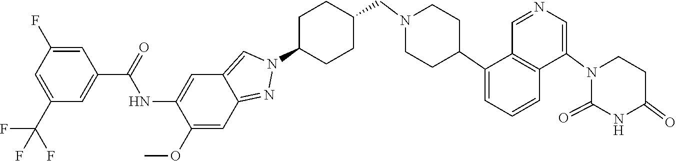 [SMILES: COc1cc2nn([C@H]3CC[C@H](CN4CCC(c5cccc6c(N7CCC(=O)NC7=O)cncc56)CC4)CC3)cc2cc1NC(=O)c1cc(F)cc(C(F)(F)F)c1] |
| I-124 | 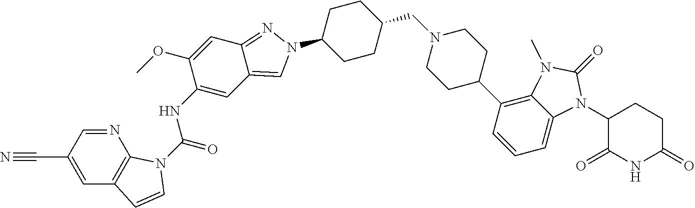 [SMILES: COc1cc2nn([C@H]3CC[C@H](CN4CCC(c5cccc6c5n(C)c(=O)n6C5CCC(=O)NC5=O)CC4)CC3)cc2cc1NC(=O)n1ccc2cc(C#N)cnc21] |
| I-125 | 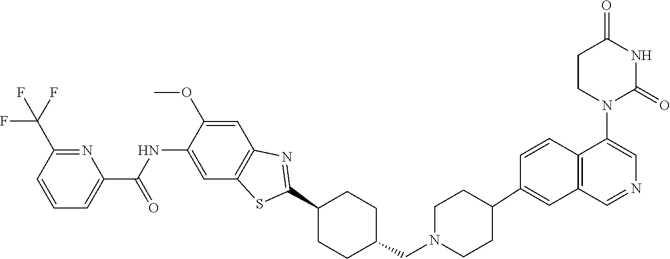 [SMILES: COc1cc2nc([C@H]3CC[C@H](CN4CCC(c5ccc6c(N7CCC(=O)NC7=O)cncc6c5)CC4)CC3)sc2cc1NC(=O)c1cccc(C(F)(F)F)n1] |
| I-126 | 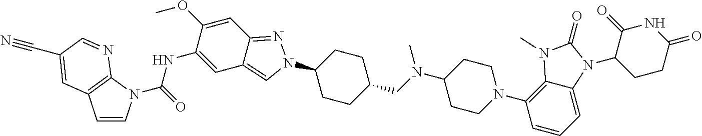 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)n1ccc2cc(C#N)cnc21] |
| I-127 | 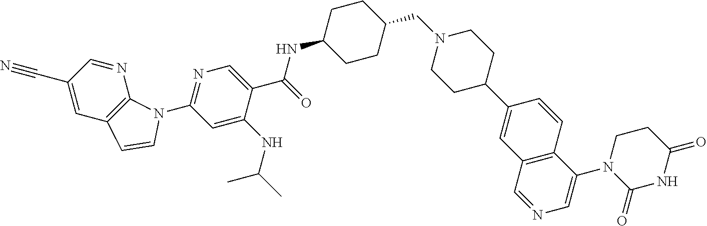 [SMILES: CC(C)Nc1cc(-n2ccc3cc(C#N)cnc32)ncc1C(=O)N[C@H]1CC[C@H](CN2CCC(c3ccc4c(N5CCC(=O)NC5=O)cncc4c3)CC2)CC1] |
| I-128 | 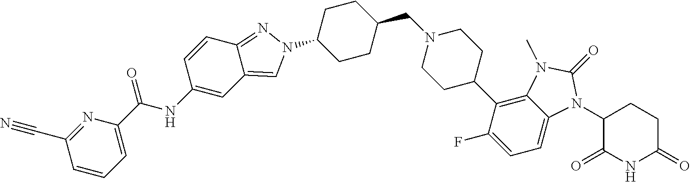 [SMILES: Cn1c(=O)n(C2CCC(=O)NC2=O)c2ccc(F)c(C3CCN(C[C@H]4CC[C@H](n5cc6cc(NC(=O)c7cccc(C#N)n7)ccc6n5)CC4)CC3)c21] |
| I-129 | 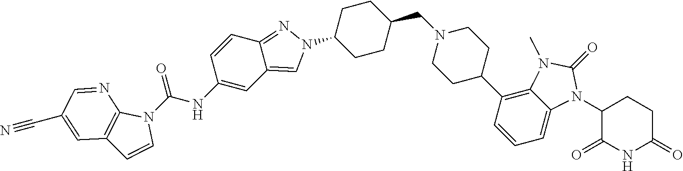 [SMILES: Cn1c(=O)n(C2CCC(=O)NC2=O)c2cccc(C3CCN(C[C@H]4CC[C@H](n5cc6cc(NC(=O)n7ccc8cc(C#N)cnc87)ccc6n5)CC4)CC3)c21] |
| I-130 | 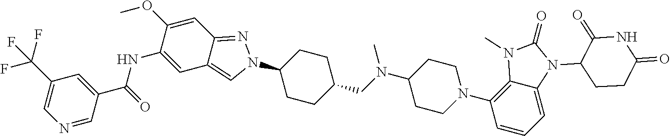 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1cncc(C(F)(F)F)c1] |
| I-131 | 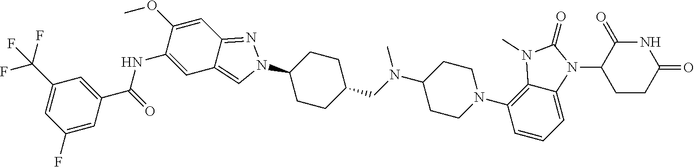 [SMILES: COc1cc2nn([C@H]3CC[C@H](CN(C)C4CCN(c5cccc6c5n(C)c(=O)n6C5CCC(=O)NC5=O)CC4)CC3)cc2cc1NC(=O)c1cc(F)cc(C(F)(F)F)c1] |
| I-132 | 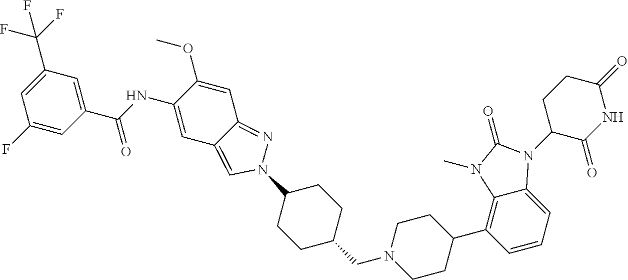 |
| I-133 | 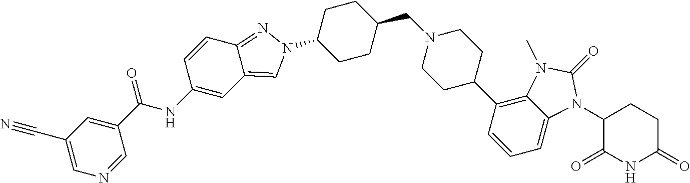 [SMILES: Cn1c(=O)n(C2CCC(=O)NC2=O)c2cccc(C3CCN(C[C@H]4CC[C@H](n5cc6cc(NC(=O)c7cncc(C#N)c7)ccc6n5)CC4)CC3)c21] |
| I-134 | 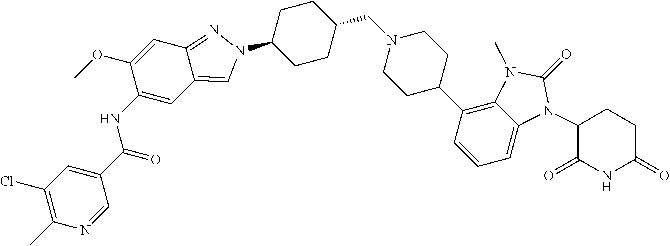 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cnc(C)c(Cl)c1] |
| I-135 | 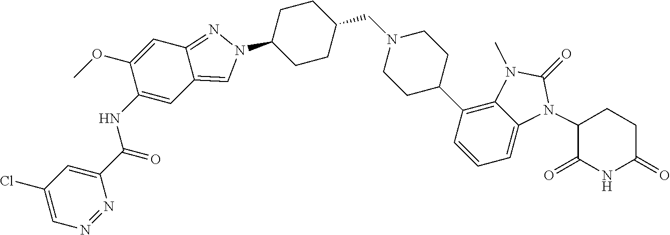 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cc(Cl)cnn1] |
| I-136 | 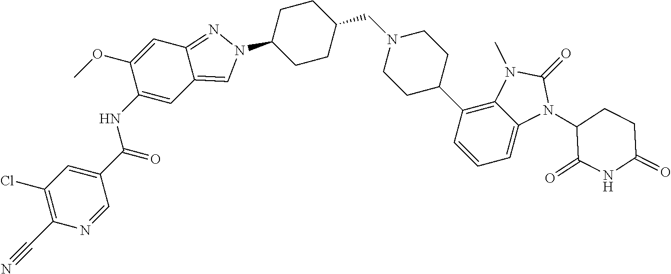 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cnc(C#N)c(Cl)c1] |
| I-137 | 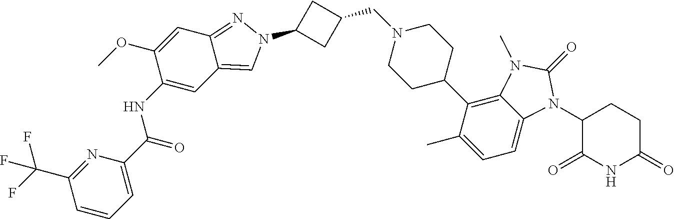 [SMILES: COc1cc2nn([C@H]3C[C@H](CN4CCC(c5c(C)ccc6c5n(C)c(=O)n6C5CCC(=O)NC5=O)CC4)C3)cc2cc1NC(=O)c1cccc(C(F)(F)F)n1] |
| I-138 | 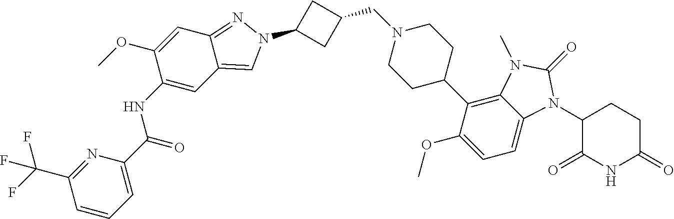 [SMILES: COc1cc2nn([C@H]3C[C@H](CN4CCC(c5c(OC)ccc6c5n(C)c(=O)n6C5CCC(=O)NC5=O)CC4)C3)cc2cc1NC(=O)c1cccc(C(F)(F)F)n1] |
| I-139 | 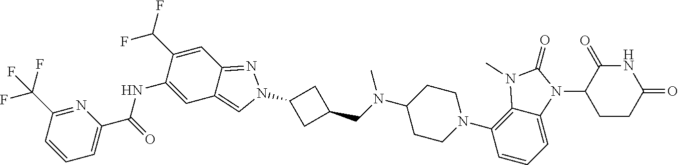 [SMILES: CN(C[C@H]1C[C@H](n2cc3cc(NC(=O)c4cccc(C(F)(F)F)n4)c(C(F)F)cc3n2)C1)C1CCN(c2cccc3c2n(C)c(=O)n3C2CCC(=O)NC2=O)CC1] |
| I-140 | 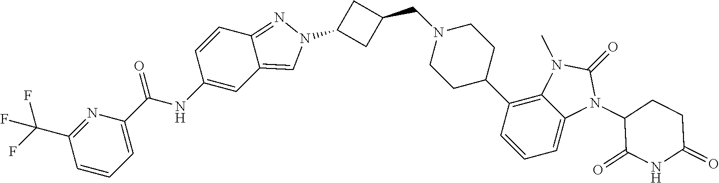 [SMILES: Cn1c(=O)n(C2CCC(=O)NC2=O)c2cccc(C3CCN(C[C@H]4C[C@H](n5cc6cc(NC(=O)c7cccc(C(F)(F)F)n7)ccc6n5)C4)CC3)c21] |
| I-141 | 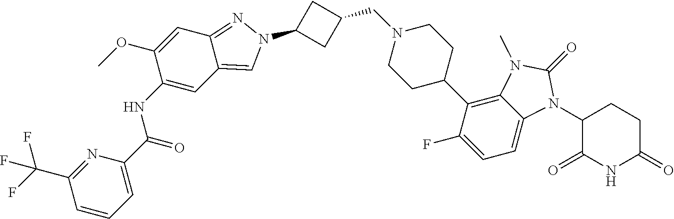 [SMILES: COc1cc2nn([C@H]3C[C@H](CN4CCC(c5c(F)ccc6c5n(C)c(=O)n6C5CCC(=O)NC5=O)CC4)C3)cc2cc1NC(=O)c1cccc(C(F)(F)F)n1] |
| I-142 | 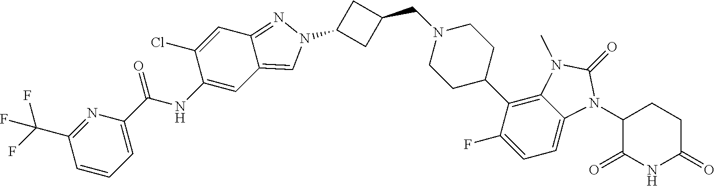 [SMILES: Cn1c(=O)n(C2CCC(=O)NC2=O)c2ccc(F)c(C3CCN(C[C@H]4C[C@H](n5cc6cc(NC(=O)c7cccc(C(F)(F)F)n7)c(Cl)cc6n5)C4)CC3)c21] |
| I-143 | 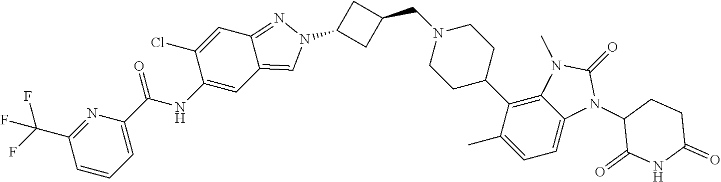 [SMILES: Cc1ccc2c(c1C1CCN(C[C@H]3C[C@H](n4cc5cc(NC(=O)c6cccc(C(F)(F)F)n6)c(Cl)cc5n4)C3)CC1)n(C)c(=O)n2C1CCC(=O)NC1=O] |
| I-144 | 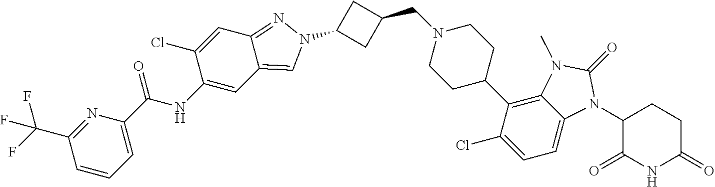 [SMILES: Cn1c(=O)n(C2CCC(=O)NC2=O)c2ccc(Cl)c(C3CCN(C[C@H]4C[C@H](n5cc6cc(NC(=O)c7cccc(C(F)(F)F)n7)c(Cl)cc6n5)C4)CC3)c21] |
| I-145 | 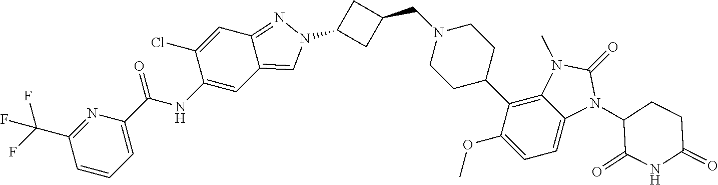 [SMILES: COc1ccc2c(c1C1CCN(C[C@H]3C[C@H](n4cc5cc(NC(=O)c6cccc(C(F)(F)F)n6)c(Cl)cc5n4)C3)CC1)n(C)c(=O)n2C1CCC(=O)NC1=O] |
| I-146 | 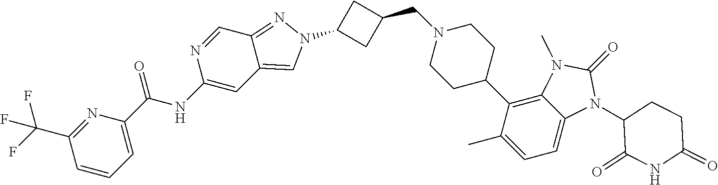 [SMILES: Cc1ccc2c(c1C1CCN(C[C@H]3C[C@H](n4cc5cc(NC(=O)c6cccc(C(F)(F)F)n6)ncc5n4)C3)CC1)n(C)c(=O)n2C1CCC(=O)NC1=O] |
| I-147 | 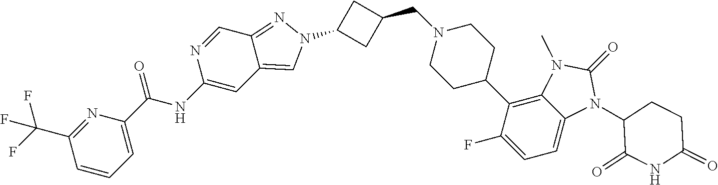 [SMILES: Cn1c(=O)n(C2CCC(=O)NC2=O)c2ccc(F)c(C3CCN(C[C@H]4C[C@H](n5cc6cc(NC(=O)c7cccc(C(F)(F)F)n7)ncc6n5)C4)CC3)c21] |
| I-148 | 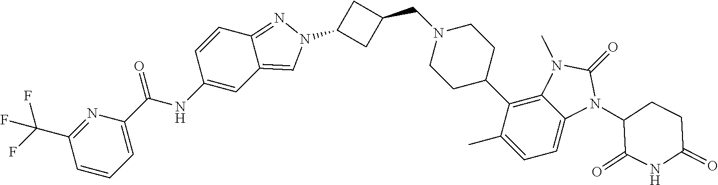 [SMILES: Cc1ccc2c(c1C1CCN(C[C@H]3C[C@H](n4cc5cc(NC(=O)c6cccc(C(F)(F)F)n6)ccc5n4)C3)CC1)n(C)c(=O)n2C1CCC(=O)NC1=O] |
| I-149 | 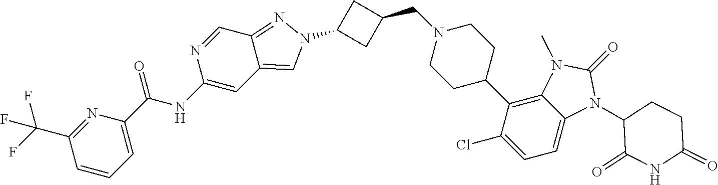 [SMILES: Cn1c(=O)n(C2CCC(=O)NC2=O)c2ccc(Cl)c(C3CCN(C[C@H]4C[C@H](n5cc6cc(NC(=O)c7cccc(C(F)(F)F)n7)ncc6n5)C4)CC3)c21] |
| I-150 | 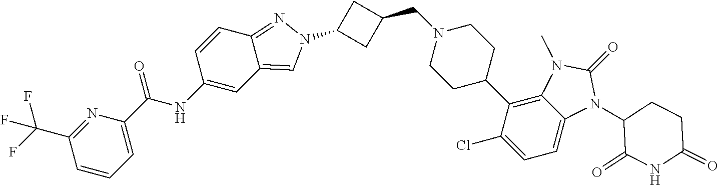 [SMILES: Cn1c(=O)n(C2CCC(=O)NC2=O)c2ccc(Cl)c(C3CCN(C[C@H]4C[C@H](n5cc6cc(NC(=O)c7cccc(C(F)(F)F)n7)ccc6n5)C4)CC3)c21] |
| I-151 | 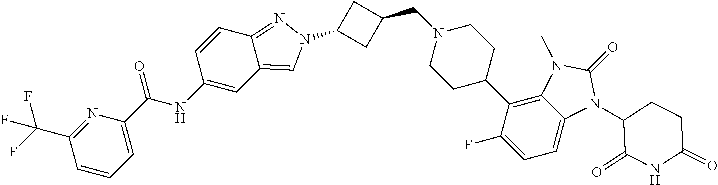 [SMILES: Cn1c(=O)n(C2CCC(=O)NC2=O)c2ccc(F)c(C3CCN(C[C@H]4C[C@H](n5cc6cc(NC(=O)c7cccc(C(F)(F)F)n7)ccc6n5)C4)CC3)c21] |
| I-152 | 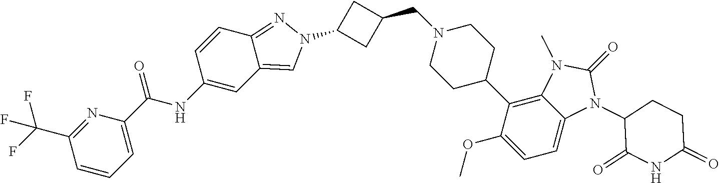 [SMILES: COc1ccc2c(c1C1CCN(C[C@H]3C[C@H](n4cc5cc(NC(=O)c6cccc(C(F)(F)F)n6)ccc5n4)C3)CC1)n(C)c(=O)n2C1CCC(=O)NC1=O] |
| I-153 | 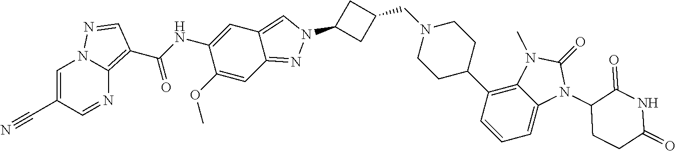 [SMILES: COc1cc2nn([C@H]3C[C@H](CN4CCC(c5cccc6c5n(C)c(=O)n6C5CCC(=O)NC5=O)CC4)C3)cc2cc1NC(=O)c1cnn2cc(C#N)cnc12] |
| I-154 | 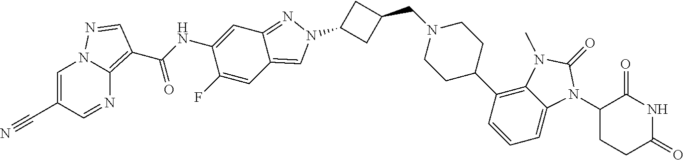 [SMILES: Cn1c(=O)n(C2CCC(=O)NC2=O)c2cccc(C3CCN(C[C@H]4C[C@H](n5cc6cc(F)c(NC(=O)c7cnn8cc(C#N)cnc78)cc6n5)C4)CC3)c21] |
| I-155 | 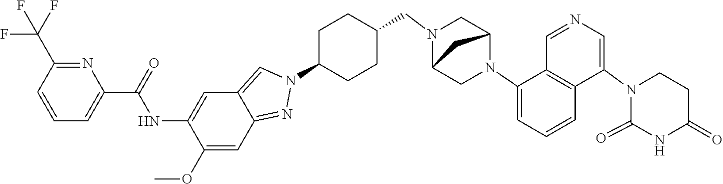 [SMILES: COc1cc2nn([C@H]3CC[C@H](CN4C[C@H]5C[C@@H]4CN5c4cccc5c(N6CCC(=O)NC6=O)cncc45)CC3)cc2cc1NC(=O)c1cccc(C(F)(F)F)n1] |
| I-156 | 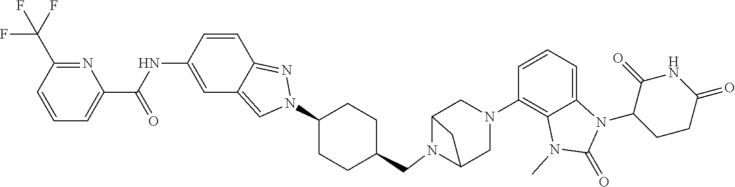 [SMILES: Cn1c(=O)n(C2CCC(=O)NC2=O)c2cccc(N3CC4CC(C3)N4C[C@H]3CC[C@@H](n4cc5cc(NC(=O)c6cccc(C(F)(F)F)n6)ccc5n4)CC3)c21] |
| I-157 | 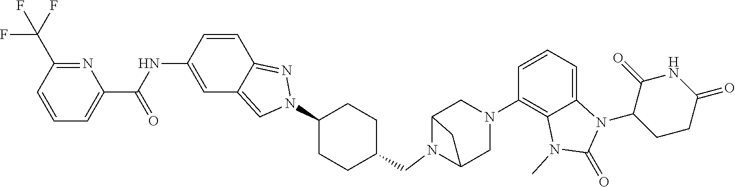 [SMILES: Cn1c(=O)n(C2CCC(=O)NC2=O)c2cccc(N3CC4CC(C3)N4C[C@H]3CC[C@H](n4cc5cc(NC(=O)c6cccc(C(F)(F)F)n6)ccc5n4)CC3)c21] |
| I-158 | 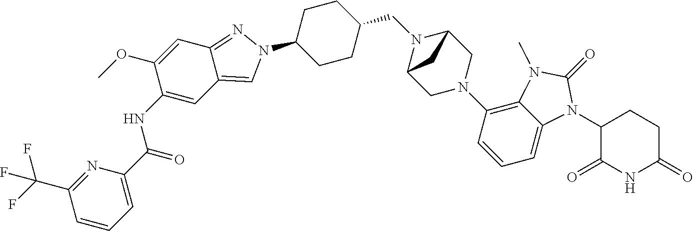 [SMILES: COc1cc2nn([C@H]3CC[C@H](CN4[C@@H]5C[C@H]4CN(c4cccc6c4n(C)c(=O)n6C4CCC(=O)NC4=O)C5)CC3)cc2cc1NC(=O)c1cccc(C(F)(F)F)n1] |
| I-159 | 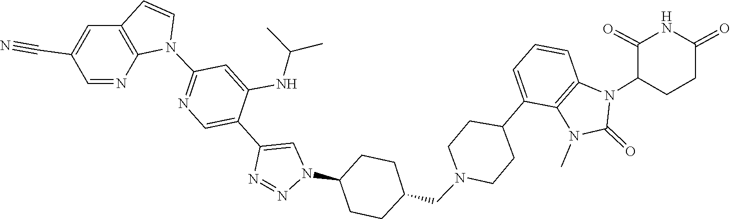 [SMILES: CC(C)Nc1cc(-n2ccc3cc(C#N)cnc32)ncc1-c1cn([C@H]2CC[C@H](CN3CCC(c4cccc5c4n(C)c(=O)n5C4CCC(=O)NC4=O)CC3)CC2)nn1] |
| I-160 | 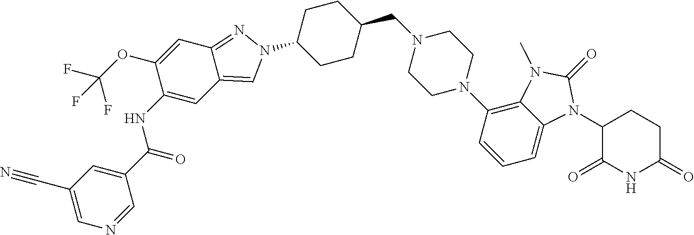 [SMILES: Cn1c(=O)n(C2CCC(=O)NC2=O)c2cccc(N3CCN(C[C@H]4CC[C@H](n5cc6cc(NC(=O)c7cncc(C#N)c7)c(OC(F)(F)F)cc6n5)CC4)CC3)c21] |
| I-161 | 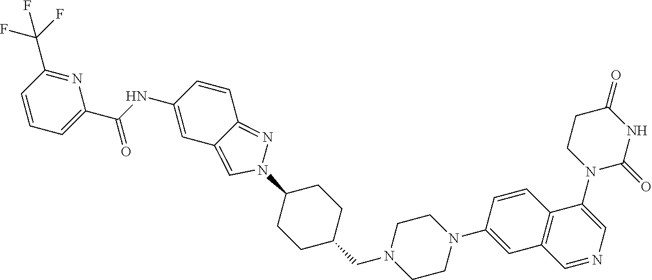 [SMILES: O=C1CCN(c2cncc3cc(N4CCN(C[C@H]5CC[C@H](n6cc7cc(NC(=O)c8cccc(C(F)(F)F)n8)ccc7n6)CC5)CC4)ccc23)C(=O)N1] |
| I-162 | 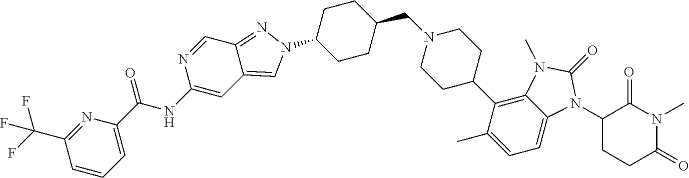 [SMILES: Cc1ccc2c(c1C1CCN(C[C@H]3CC[C@H](n4cc5cc(NC(=O)c6cccc(C(F)(F)F)n6)ncc5n4)CC3)CC1)n(C)c(=O)n2C1CCC(=O)N(C)C1=O] |
| I-163 | 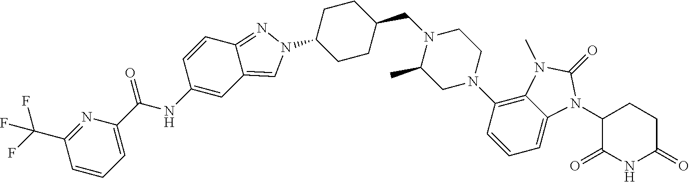 [SMILES: C[C@@H]1CN(c2cccc3c2n(C)c(=O)n3C2CCC(=O)NC2=O)CCN1C[C@H]1CC[C@H](n2cc3cc(NC(=O)c4cccc(C(F)(F)F)n4)ccc3n2)CC1] |
| I-164 | 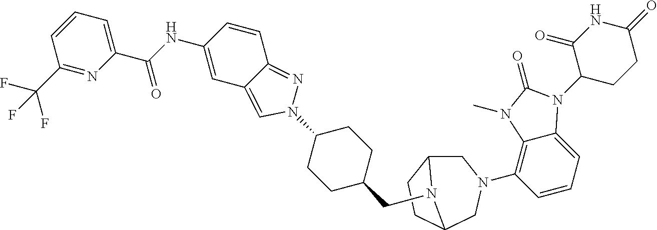 [SMILES: Cn1c(=O)n(C2CCC(=O)NC2=O)c2cccc(N3CC4CCC(C3)N4C[C@H]3CC[C@H](n4cc5cc(NC(=O)c6cccc(C(F)(F)F)n6)ccc5n4)CC3)c21] |
| I-165 | 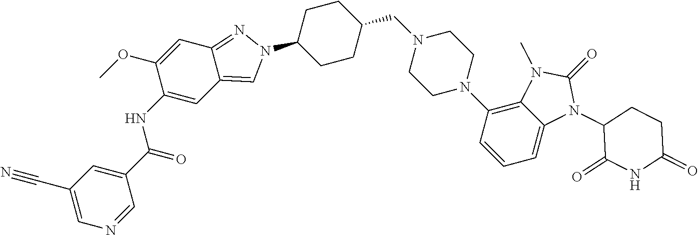 [SMILES: COc1cc2nn([C@H]3CC[C@H](CN4CCN(c5cccc6c5n(C)c(=O)n6C5CCC(=O)NC5=O)CC4)CC3)cc2cc1NC(=O)c1cncc(C#N)c1] |
| I-166 | 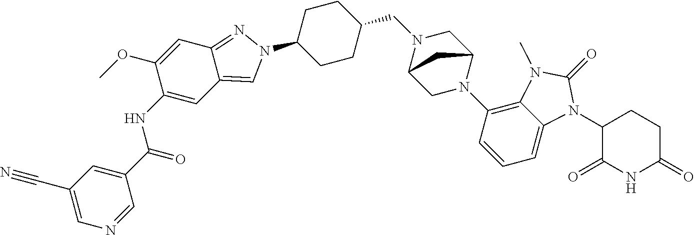 [SMILES: COc1cc2nn([C@H]3CC[C@H](CN4C[C@H]5C[C@@H]4CN5c4cccc5c4n(C)c(=O)n5C4CCC(=O)NC4=O)CC3)cc2cc1NC(=O)c1cncc(C#N)c1] |
| I-167 | 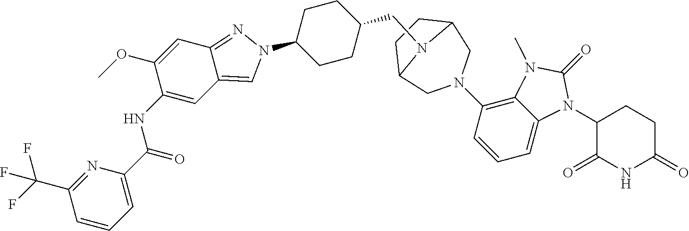 [SMILES: COc1cc2nn([C@H]3CC[C@H](CN4C5CCC4CN(c4cccc6c4n(C)c(=O)n6C4CCC(=O)NC4=O)C5)CC3)cc2cc1NC(=O)c1cccc(C(F)(F)F)n1] |
| I-168 | 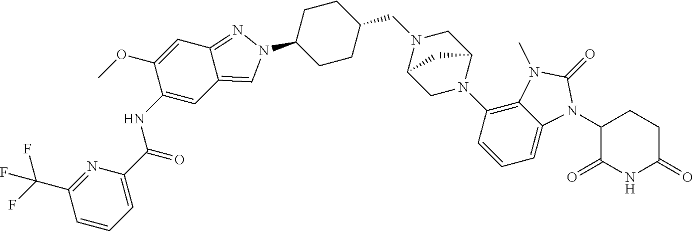 [SMILES: COc1cc2nn([C@H]3CC[C@H](CN4C[C@@H]5C[C@H]4CN5c4cccc5c4n(C)c(=O)n5C4CCC(=O)NC4=O)CC3)cc2cc1NC(=O)c1cccc(C(F)(F)F)n1] |
| I-169 | 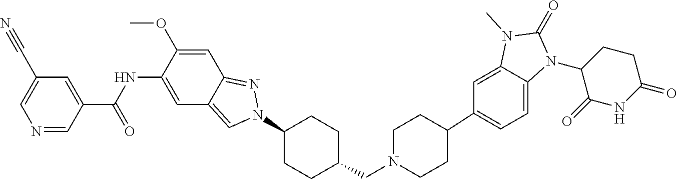 [SMILES: COc1cc2nn([C@H]3CC[C@H](CN4CCC(c5ccc6c(c5)n(C)c(=O)n6C5CCC(=O)NC5=O)CC4)CC3)cc2cc1NC(=O)c1cncc(C#N)c1] |
| I-170 | 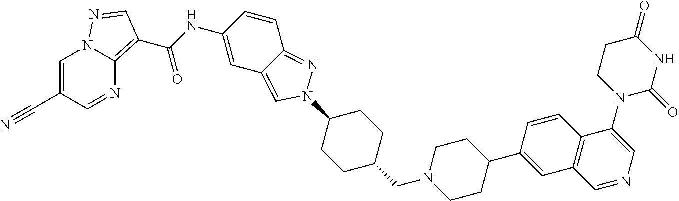 [SMILES: N#Cc1cnc2c(C(=O)Nc3ccc4nn([C@H]5CC[C@H](CN6CCC(c7ccc8c(N9CCC(=O)NC9=O)cncc8c7)CC6)CC5)cc4c3)cnn2c1] |
| I-171 | 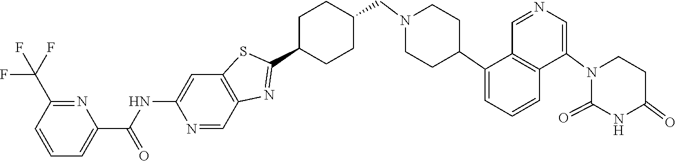 [SMILES: O=C1CCN(c2cncc3c(C4CCN(C[C@H]5CC[C@H](c6nc7cnc(NC(=O)c8cccc(C(F)(F)F)n8)cc7s6)CC5)CC4)cccc23)C(=O)N1] |
| I-172 | 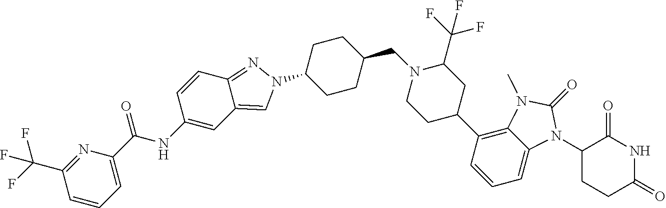 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)ccc6n5)CC4)C(C(F)(F)F)C3)c21] |
| I-173 | 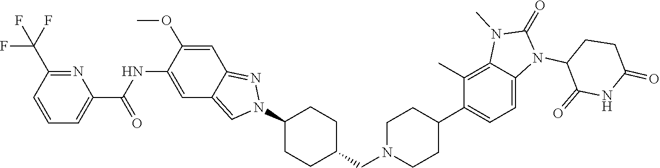 [SMILES: COc1cc2nn([C@H]3CC[C@H](CN4CCC(c5ccc6c(c5C)n(C)c(=O)n6C5CCC(=O)NC5=O)CC4)CC3)cc2cc1NC(=O)c1cccc(C(F)(F)F)n1] |
| I-174 | 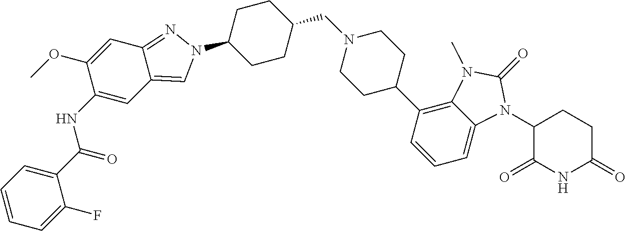 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1ccccc1F] |
| I-175 | 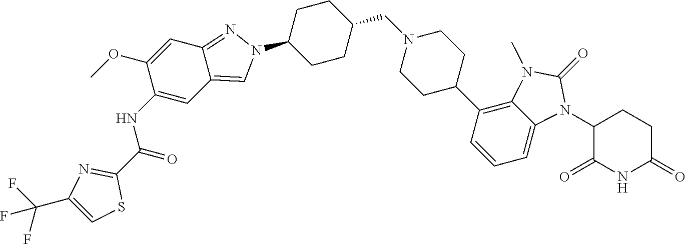 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1nc(C(F)(F)F)cs1] |
| I-176 | 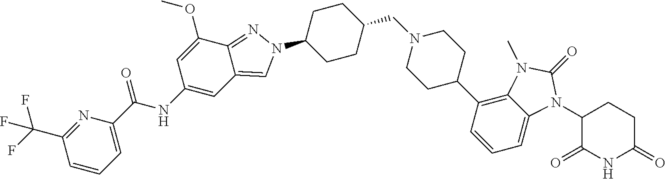 [SMILES: COc1cc(NC(=O)c2cccc(C(F)(F)F)n2)cc2cn([C@H]3CC[C@H](CN4CCC(c5cccc6c5n(C)c(=O)n6C5CCC(=O)NC5=O)CC4)CC3)nc12] |
| I-177 | 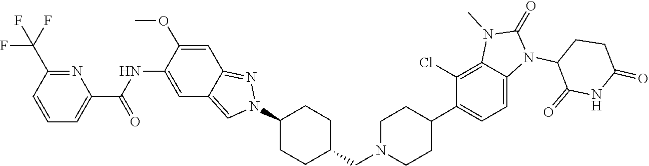 [SMILES: COc1cc2nn([C@H]3CC[C@H](CN4CCC(c5ccc6c(c5Cl)n(C)c(=O)n6C5CCC(=O)NC5=O)CC4)CC3)cc2cc1NC(=O)c1cccc(C(F)(F)F)n1] |
| I-178 | 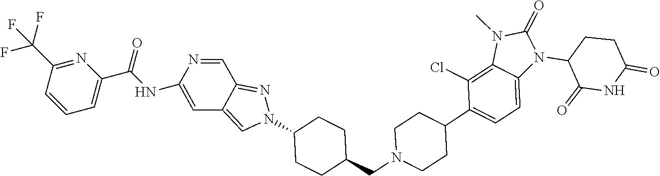 [SMILES: Cn1c(=O)n(C2CCC(=O)NC2=O)c2ccc(C3CCN(C[C@H]4CC[C@H](n5cc6cc(NC(=O)c7cccc(C(F)(F)F)n7)ncc6n5)CC4)CC3)c(Cl)c21] |
| I-179 | 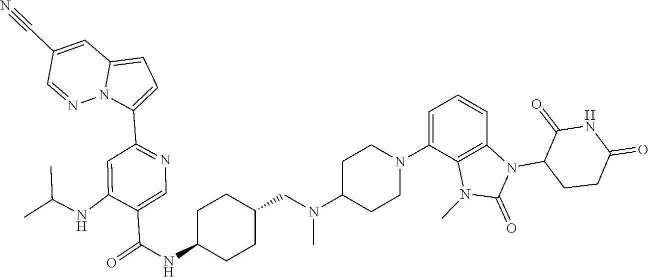 [SMILES: CC(C)Nc1cc(-c2ccc3cc(C#N)cnn23)ncc1C(=O)N[C@H]1CC[C@H](CN(C)C2CCN(c3cccc4c3n(C)c(=O)n4C3CCC(=O)NC3=O)CC2)CC1] |
| I-180 | 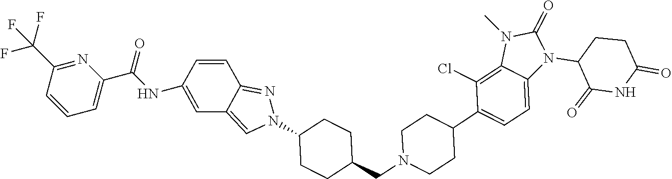 [SMILES: Cn1c(=O)n(C2CCC(=O)NC2=O)c2ccc(C3CCN(C[C@H]4CC[C@H](n5cc6cc(NC(=O)c7cccc(C(F)(F)F)n7)ccc6n5)CC4)CC3)c(Cl)c21] |
| I-181 | 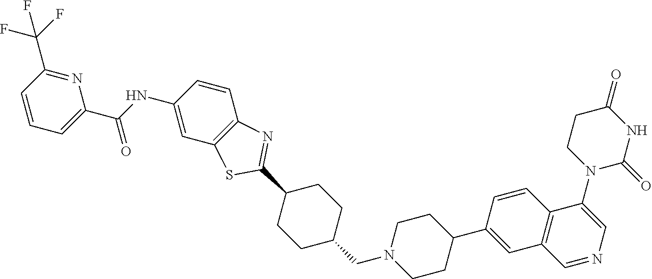 [SMILES: O=C1CCN(c2cncc3cc(C4CCN(C[C@H]5CC[C@H](c6nc7ccc(NC(=O)c8cccc(C(F)(F)F)n8)cc7s6)CC5)CC4)ccc23)C(=O)N1] |
| I-182 | 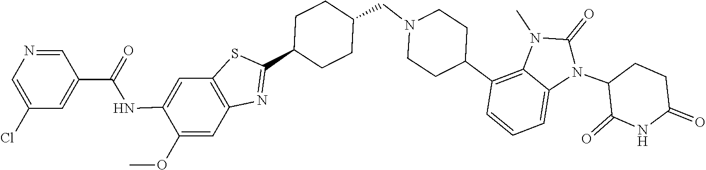 [SMILES: COc1cc2nc([C@H]3CC[C@H](CN4CCC(c5cccc6c5n(C)c(=O)n6C5CCC(=O)NC5=O)CC4)CC3)sc2cc1NC(=O)c1cncc(Cl)c1] |
| I-183 | 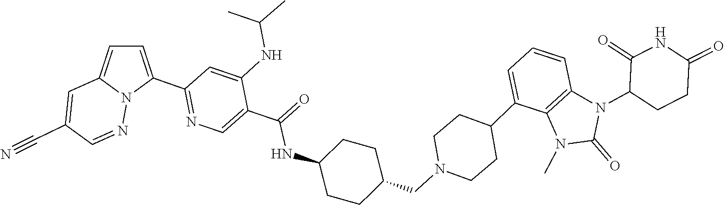 [SMILES: CC(C)Nc1cc(-c2ccc3cc(C#N)cnn23)ncc1C(=O)N[C@H]1CC[C@H](CN2CCC(c3cccc4c3n(C)c(=O)n4C3CCC(=O)NC3=O)CC2)CC1] |
| I-184 | 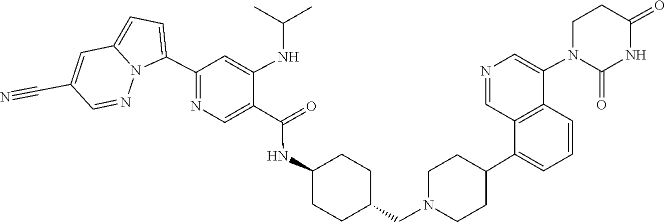 [SMILES: CC(C)Nc1cc(-c2ccc3cc(C#N)cnn23)ncc1C(=O)N[C@H]1CC[C@H](CN2CCC(c3cccc4c(N5CCC(=O)NC5=O)cncc34)CC2)CC1] |
| I-185 | 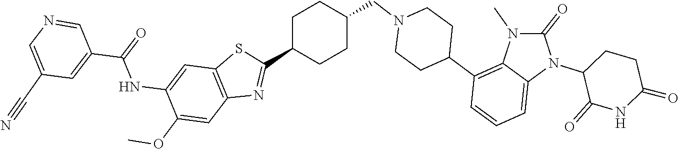 [SMILES: COc1cc2nc([C@H]3CC[C@H](CN4CCC(c5cccc6c5n(C)c(=O)n6C5CCC(=O)NC5=O)CC4)CC3)sc2cc1NC(=O)c1cncc(C#N)c1] |
| I-186 | 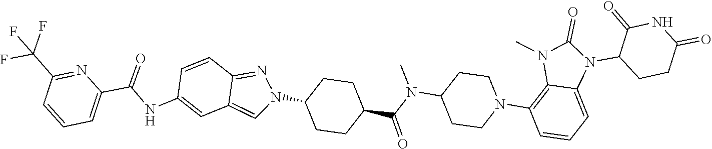 [SMILES: CN(C(=O)[C@H]1CC[C@H](n2cc3cc(NC(=O)c4cccc(C(F)(F)F)n4)ccc3n2)CC1)C1CCN(c2cccc3c2n(C)c(=O)n3C2CCC(=O)NC2=O)CC1] |
| I-187 | 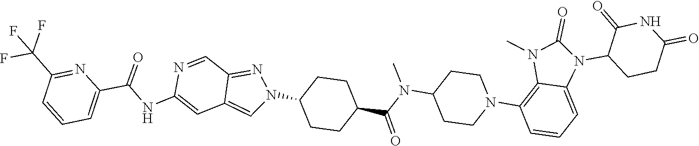 [SMILES: CN(C(=O)[C@H]1CC[C@H](n2cc3cc(NC(=O)c4cccc(C(F)(F)F)n4)ncc3n2)CC1)C1CCN(c2cccc3c2n(C)c(=O)n3C2CCC(=O)NC2=O)CC1] |
| I-188 | 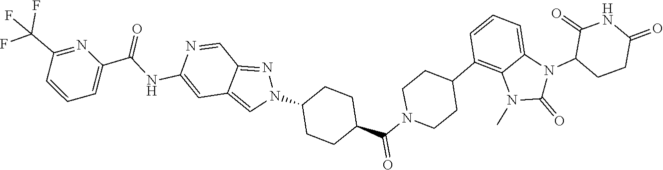 [SMILES: Cn1c(=O)n(C2CCC(=O)NC2=O)c2cccc(C3CCN(C(=O)[C@H]4CC[C@H](n5cc6cc(NC(=O)c7cccc(C(F)(F)F)n7)ncc6n5)CC4)CC3)c21] |
| I-189 | 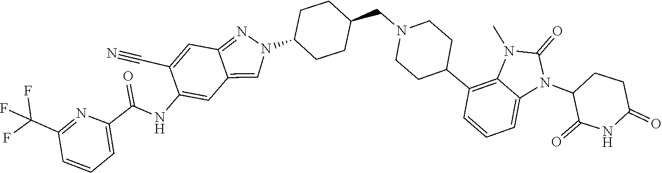 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)c(C#N)cc6n5)CC4)CC3)c21] |
| I-190 | 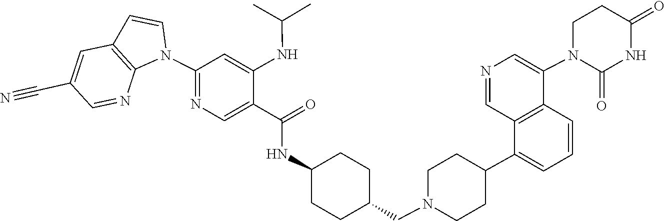 [SMILES: CC(C)Nc1cc(-n2ccc3cc(C#N)cnc32)ncc1C(=O)N[C@H]1CC[C@H](CN2CCC(c3cccc4c(N5CCC(=O)NC5=O)cncc34)CC2)CC1] |
| I-191 | 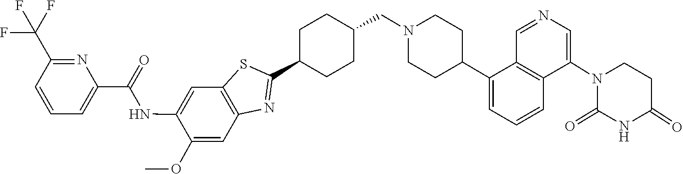 [SMILES: COc1cc2nc([C@H]3CC[C@H](CN4CCC(c5cccc6c(N7CCC(=O)NC7=O)cncc56)CC4)CC3)sc2cc1NC(=O)c1cccc(C(F)(F)F)n1] |
| I-193 | 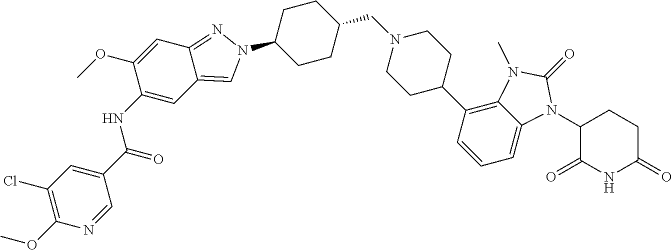 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cnc(OC)c(Cl)c1] |
| I-194 | 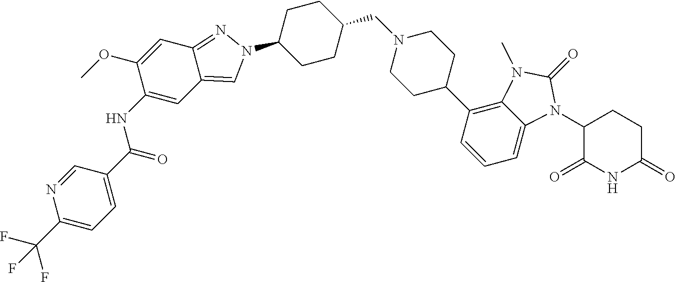 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1ccc(C(F)(F)F)nc1] |
| I-195 | 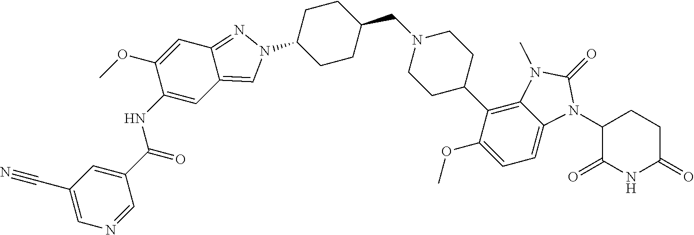 [SMILES: COc1cc2nn([C@H]3CC[C@H](CN4CCC(c5c(OC)ccc6c5n(C)c(=O)n6C5CCC(=O)NC5=O)CC4)CC3)cc2cc1NC(=O)c1cncc(C#N)c1] |
| I-196 | 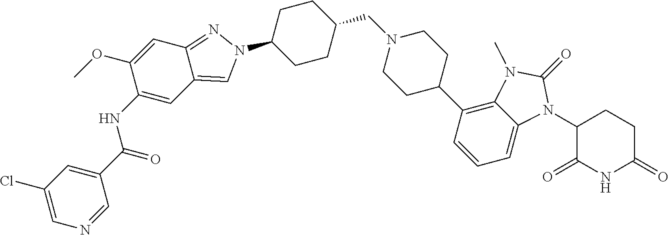 [SMILES: COc1cc2nn([C@H]3CC[C@H](CN4CCC(c5cccc6c5n(C)c(=O)n6C5CCC(=O)NC5=O)CC4)CC3)cc2cc1NC(=O)c1cncc(Cl)c1] |
| I-197 | 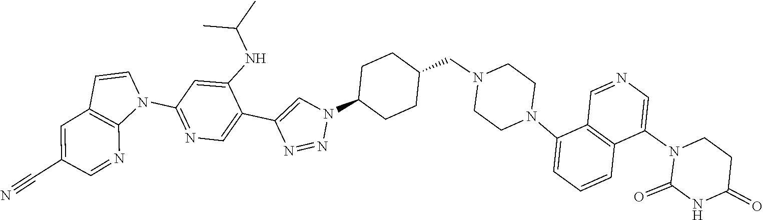 [SMILES: CC(C)Nc1cc(-n2ccc3cc(C#N)cnc32)ncc1-c1cn([C@H]2CC[C@H](CN3CCN(c4cccc5c(N6CCC(=O)NC6=O)cncc45)CC3)CC2)nn1] |
| I-198 | 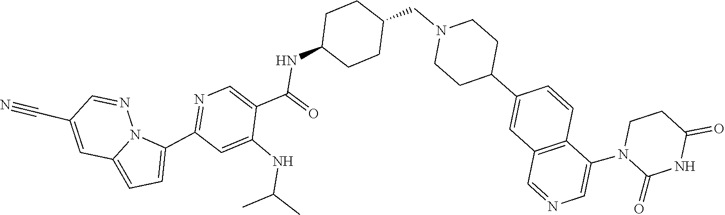 [SMILES: CC(C)Nc1cc(-c2ccc3cc(C#N)cnn23)ncc1C(=O)N[C@H]1CC[C@H](CN2CCC(c3ccc4c(N5CCC(=O)NC5=O)cncc4c3)CC2)CC1] |
| I-199 | 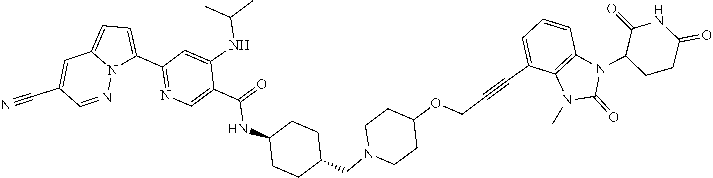 [SMILES: CC(C)Nc1cc(-c2ccc3cc(C#N)cnn23)ncc1C(=O)N[C@H]1CC[C@H](CN2CCC(OCC#Cc3cccc4c3n(C)c(=O)n4C3CCC(=O)NC3=O)CC2)CC1] |
| I-200 | 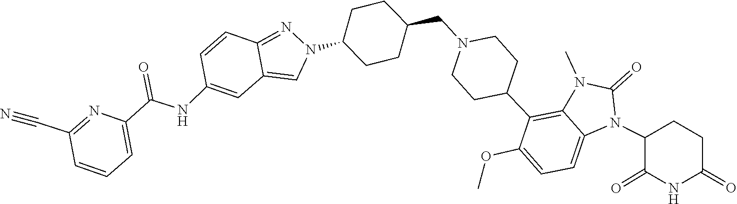 [SMILES: COc1ccc2c(c1C1CCN(C[C@H]3CC[C@H](n4cc5cc(NC(=O)c6cccc(C#N)n6)ccc5n4)CC3)CC1)n(C)c(=O)n2C1CCC(=O)NC1=O] |
| I-201 | 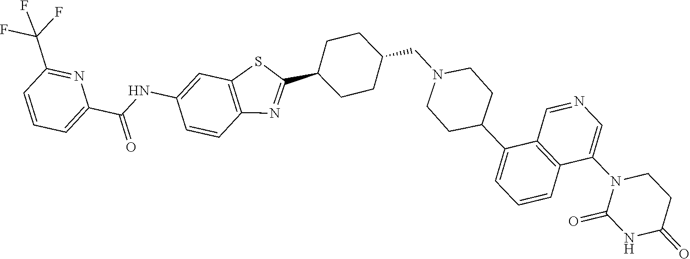 [SMILES: O=C1CCN(c2cncc3c(C4CCN(C[C@H]5CC[C@H](c6nc7ccc(NC(=O)c8cccc(C(F)(F)F)n8)cc7s6)CC5)CC4)cccc23)C(=O)N1] |
| I-202 | 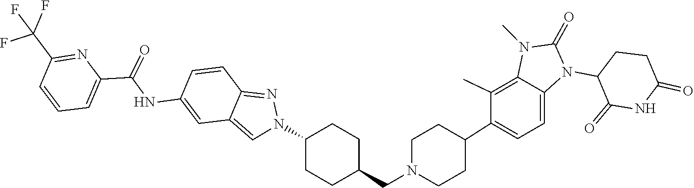 [SMILES: Cc1c(C2CCN(C[C@H]3CC[C@H](n4cc5cc(NC(=O)c6cccc(C(F)(F)F)n6)ccc5n4)CC3)CC2)ccc2c1n(C)c(=O)n2C1CCC(=O)NC1=O] |
| I-203 | 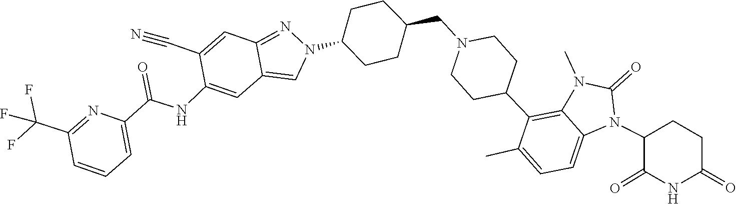 [SMILES: Cc1ccc2c(c1C1CCN(C[C@H]3CC[C@H](n4cc5cc(NC(=O)c6cccc(C(F)(F)F)n6)c(C#N)cc5n4)CC3)CC1)n(C)c(=O)n2C1CCC(=O)NC1=O] |
| I-204 | 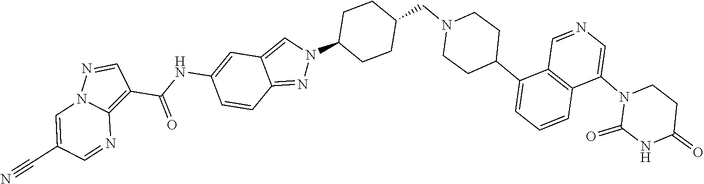 [SMILES: N#Cc1cnc2c(C(=O)Nc3ccc4nn([C@H]5CC[C@H](CN6CCC(c7cccc8c(N9CCC(=O)NC9=O)cncc78)CC6)CC5)cc4c3)cnn2c1] |
| I-205 | 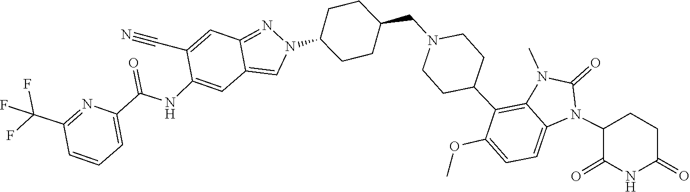 [SMILES: COc1ccc2c(c1C1CCN(C[C@H]3CC[C@H](n4cc5cc(NC(=O)c6cccc(C(F)(F)F)n6)c(C#N)cc5n4)CC3)CC1)n(C)c(=O)n2C1CCC(=O)NC1=O] |
| I-206 | 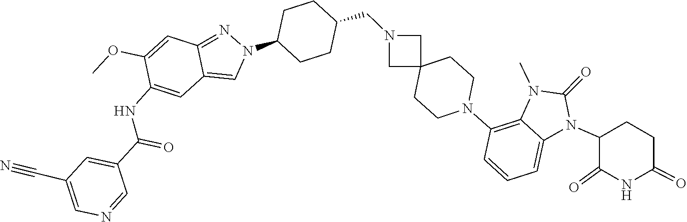 [SMILES: COc1cc2nn([C@H]3CC[C@H](CN4CC5(CCN(c6cccc7c6n(C)c(=O)n7C6CCC(=O)NC6=O)CC5)C4)CC3)cc2cc1NC(=O)c1cncc(C#N)c1] |
| I-207 | 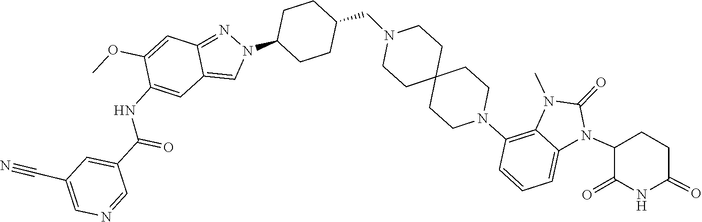 [SMILES: COc1cc2nn([C@H]3CC[C@H](CN4CCC5(CC4)CCN(c4cccc6c4n(C)c(=O)n6C4CCC(=O)NC4=O)CC5)CC3)cc2cc1NC(=O)c1cncc(C#N)c1] |
| I-208 | 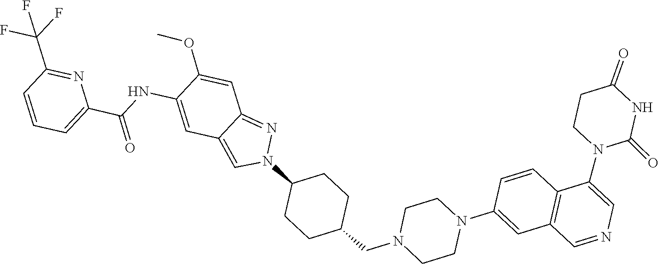 [SMILES: COc1cc2nn([C@H]3CC[C@H](CN4CCN(c5ccc6c(N7CCC(=O)NC7=O)cncc6c5)CC4)CC3)cc2cc1NC(=O)c1cccc(C(F)(F)F)n1] |
| I-209 | 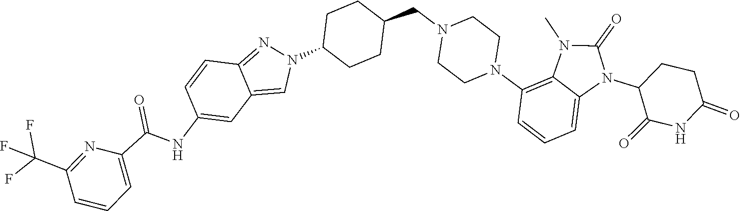 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)ccc6n5)CC4)CC3)c21] |
| I-210 | 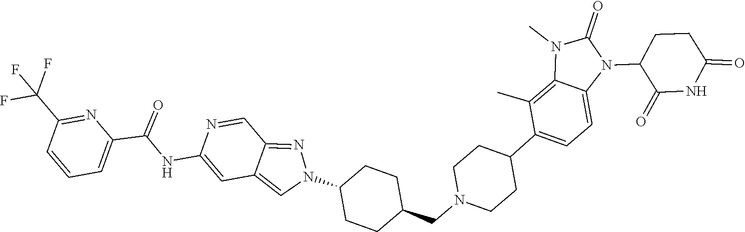 [SMILES: Cc1c(C2CCN(C[C@H]3CC[C@H](n4cc5cc(NC(=O)c6cccc(C(F)(F)F)n6)ncc5n4)CC3)CC2)ccc2c1n(C)c(=O)n2C1CCC(=O)NC1=O] |
| I-211 | 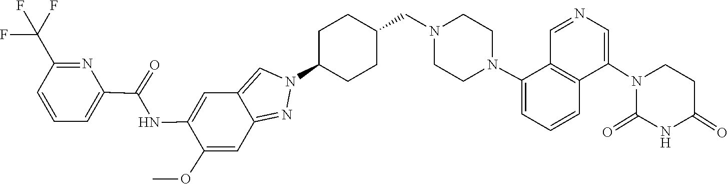 [SMILES: COc1cc2nn([C@H]3CC[C@H](CN4CCN(c5cccc6c(N7CCC(=O)NC7=O)cncc56)CC4)CC3)cc2cc1NC(=O)c1cccc(C(F)(F)F)n1] |
| I-212 | 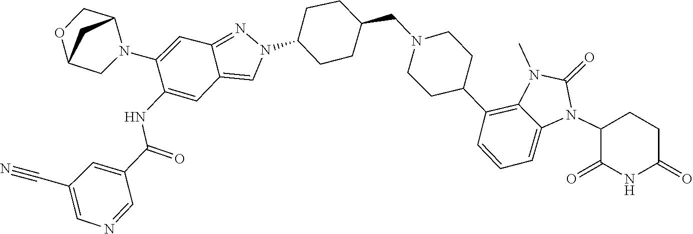 [SMILES: Cn1c(=O)n(C2CCC(=O)NC2=O)c2cccc(C3CCN(C[C@H]4CC[C@H](n5cc6cc(NC(=O)c7cncc(C#N)c7)c(N7C[C@H]8C[C@@H]7CO8)cc6n5)CC4)CC3)c21] |
| I-213 | 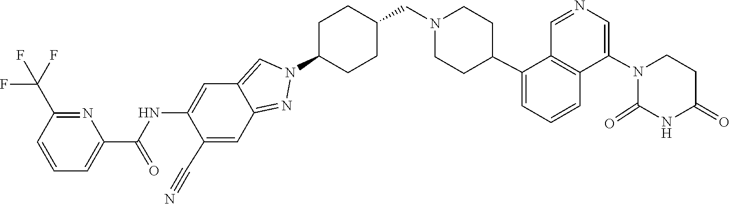 [SMILES: N#Cc1cc2nn([C@H]3CC[C@H](CN4CCC(c5cccc6c(N7CCC(=O)NC7=O)cncc56)CC4)CC3)cc2cc1NC(=O)c1cccc(C(F)(F)F)n1] |
| I-214 | 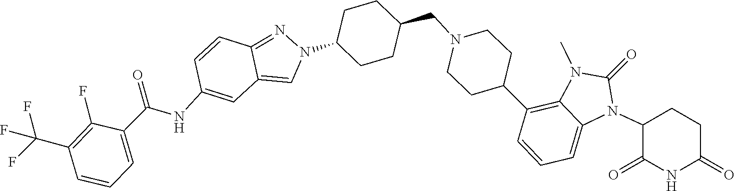 [SMILES: Cn1c(=O)n(C2CCC(=O)NC2=O)c2cccc(C3CCN(C[C@H]4CC[C@H](n5cc6cc(NC(=O)c7cccc(C(F)(F)F)c7F)ccc6n5)CC4)CC3)c21] |
| I-215 | 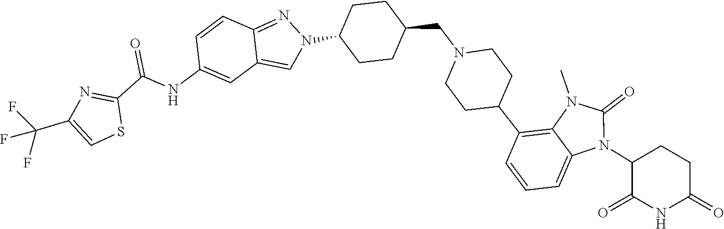 [SMILES: Cn1c(=O)n(C2CCC(=O)NC2=O)c2cccc(C3CCN(C[C@H]4CC[C@H](n5cc6cc(NC(=O)c7nc(C(F)(F)F)cs7)ccc6n5)CC4)CC3)c21] |
| I-216 | 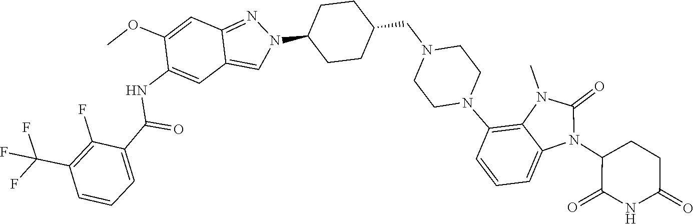 [SMILES: COc1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)c1F] |
| I-217 | 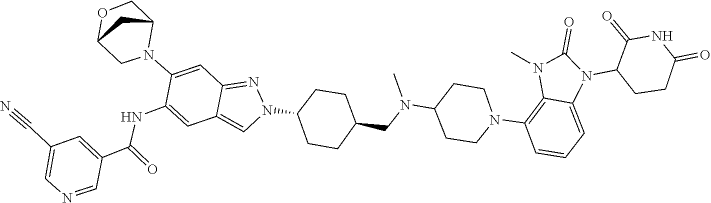 [SMILES: CN(C[C@H]1CC[C@H](n2cc3cc(NC(=O)c4cncc(C#N)c4)c(N4C[C@H]5C[C@@H]4CO5)cc3n2)CC1)C1CCN(c2cccc3c2n(C)c(=O)n3C2CCC(=O)NC2=O)CC1] |
| I-218 | 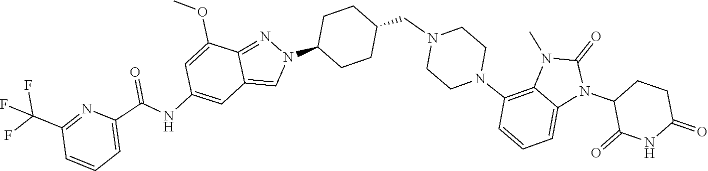 [SMILES: COc1cc(NC(=O)c2cccc(C(F)(F)F)n2)cc2cn([C@H]3CC[C@H](CN4CCN(c5cccc6c5n(C)c(=O)n6C5CCC(=O)NC5=O)CC4)CC3)nc12] |
| I-219 | 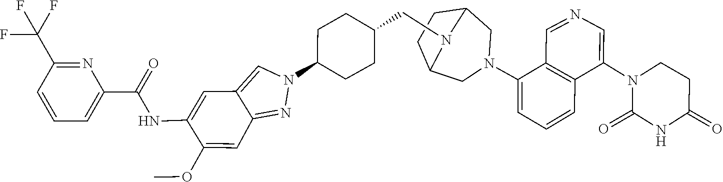 [SMILES: COc1cc2nn([C@H]3CC[C@H](CN4C5CCC4CN(c4cccc6c(N7CCC(=O)NC7=O)cncc46)C5)CC3)cc2cc1NC(=O)c1cccc(C(F)(F)F)n1] |
| I-220 | 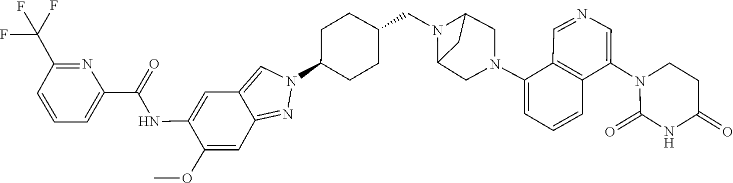 [SMILES: COc1cc2nn([C@H]3CC[C@H](CN4C5CC4CN(c4cccc6c(N7CCC(=O)NC7=O)cncc46)C5)CC3)cc2cc1NC(=O)c1cccc(C(F)(F)F)n1] |
| I-221 | 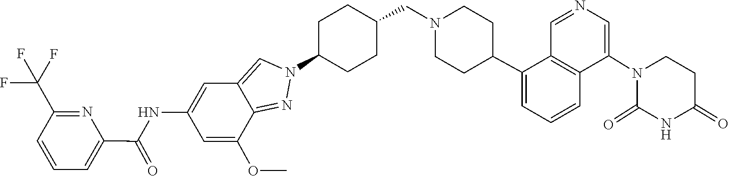 [SMILES: COc1cc(NC(=O)c2cccc(C(F)(F)F)n2)cc2cn([C@H]3CC[C@H](CN4CCC(c5cccc6c(N7CCC(=O)NC7=O)cncc56)CC4)CC3)nc12] |
| I-222 | 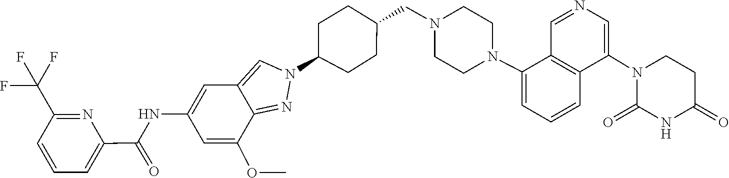 [SMILES: COc1cc(NC(=O)c2cccc(C(F)(F)F)n2)cc2cn([C@H]3CC[C@H](CN4CCN(c5cccc6c(N7CCC(=O)NC7=O)cncc56)CC4)CC3)nc12] |
| I-223 | 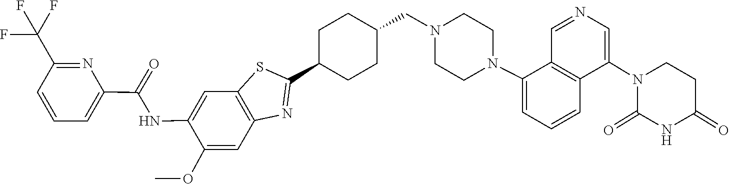 [SMILES: COc1cc2nc([C@H]3CC[C@H](CN4CCN(c5cccc6c(N7CCC(=O)NC7=O)cncc56)CC4)CC3)sc2cc1NC(=O)c1cccc(C(F)(F)F)n1] |
| I-224 | 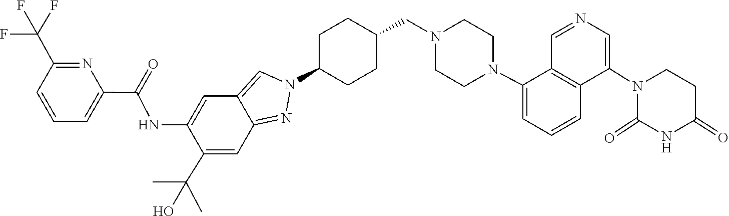 [SMILES: CC(C)(O)c1cc2nn([C@H]3CC[C@H](CN4CCN(c5cccc6c(N7CCC(=O)NC7=O)cncc56)CC4)CC3)cc2cc1NC(=O)c1cccc(C(F)(F)F)n1] |
| I-225 | 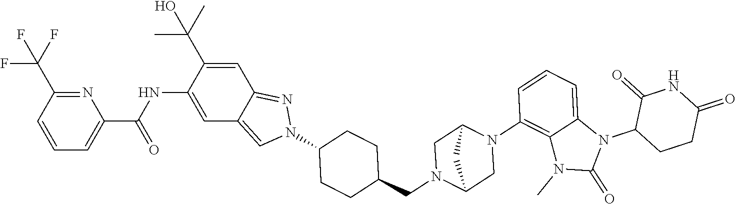 [SMILES: Cn1c(=O)n(C2CCC(=O)NC2=O)c2cccc(N3C[C@@H]4C[C@H]3CN4C[C@H]3CC[C@H](n4cc5cc(NC(=O)c6cccc(C(F)(F)F)n6)c(C(C)(C)O)cc5n4)CC3)c21] |
| I-226 | 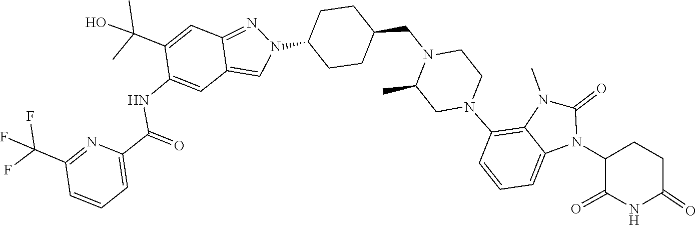 [SMILES: C[C@@H]1CN(c2cccc3c2n(C)c(=O)n3C2CCC(=O)NC2=O)CCN1C[C@H]1CC[C@H](n2cc3cc(NC(=O)c4cccc(C(F)(F)F)n4)c(C(C)(C)O)cc3n2)CC1] |
| I-227 | 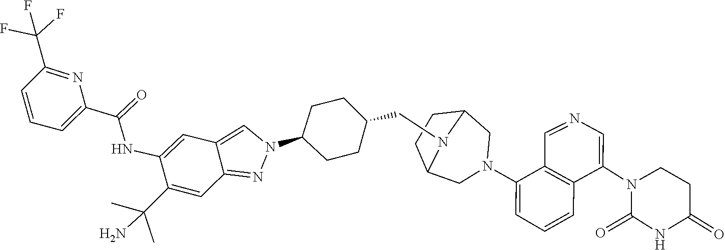 [SMILES: CC(C)(N)c1cc2nn([C@H]3CC[C@H](CN4C5CCC4CN(c4cccc6c(N7CCC(=O)NC7=O)cncc46)C5)CC3)cc2cc1NC(=O)c1cccc(C(F)(F)F)n1] |
| I-228 | 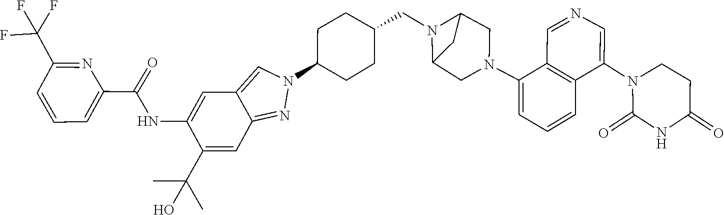 [SMILES: CC(C)(O)c1cc2nn([C@H]3CC[C@H](CN4C5CC4CN(c4cccc6c(N7CCC(=O)NC7=O)cncc46)C5)CC3)cc2cc1NC(=O)c1cccc(C(F)(F)F)n1] |
| I-229 | 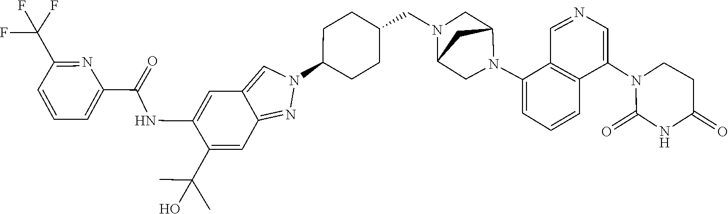 [SMILES: CC(C)(O)c1cc2nn([C@H]3CC[C@H](CN4C[C@H]5C[C@@H]4CN5c4cccc5c(N6CCC(=O)NC6=O)cncc45)CC3)cc2cc1NC(=O)c1cccc(C(F)(F)F)n1] |
| I-230 | 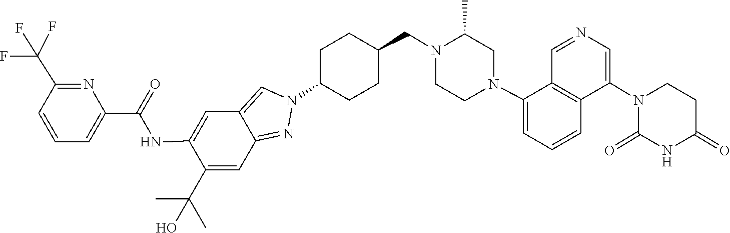 [SMILES: C[C@@H]1CN(c2cccc3c(N4CCC(=O)NC4=O)cncc23)CCN1C[C@H]1CC[C@H](n2cc3cc(NC(=O)c4cccc(C(F)(F)F)n4)c(C(C)(C)O)cc3n2)CC1] |
| I-231 | 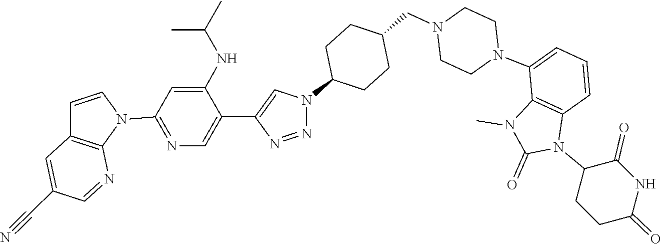 [SMILES: CC(C)Nc1cc(-n2ccc3cc(C#N)cnc32)ncc1-c1cn([C@H]2CC[C@H](CN3CCN(c4cccc5c4n(C)c(=O)n5C4CCC(=O)NC4=O)CC3)CC2)nn1] |
| I-232 | 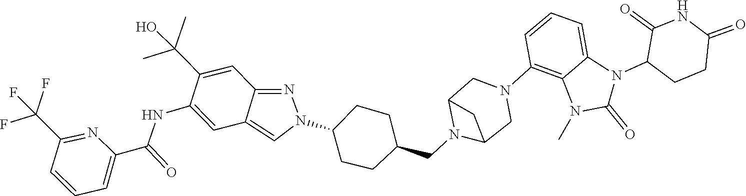 [SMILES: Cn1c(=O)n(C2CCC(=O)NC2=O)c2cccc(N3CC4CC(C3)N4C[C@H]3CC[C@H](n4cc5cc(NC(=O)c6cccc(C(F)(F)F)n6)c(C(C)(C)O)cc5n4)CC3)c21] |
| I-233 | 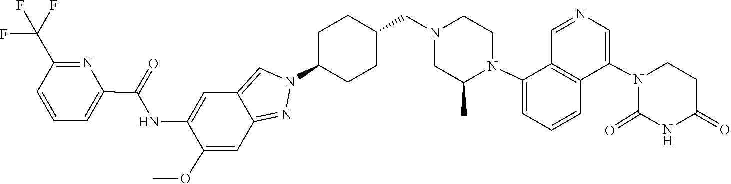 [SMILES: COc1cc2nn([C@H]3CC[C@H](CN4CCN(c5cccc6c(N7CCC(=O)NC7=O)cncc56)[C@@H](C)C4)CC3)cc2cc1NC(=O)c1cccc(C(F)(F)F)n1] |
| I-234 | 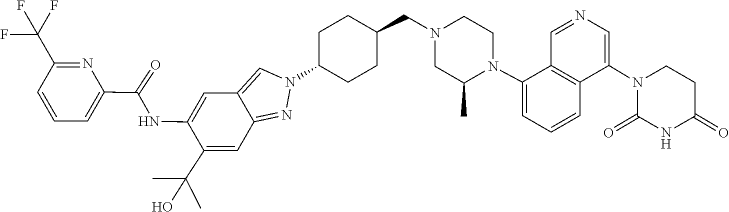 [SMILES: C[C@H]1CN(C[C@H]2CC[C@H](n3cc4cc(NC(=O)c5cccc(C(F)(F)F)n5)c(C(C)(C)O)cc4n3)CC2)CCN1c1cccc2c(N3CCC(=O)NC3=O)cncc12] |
| I-235 | 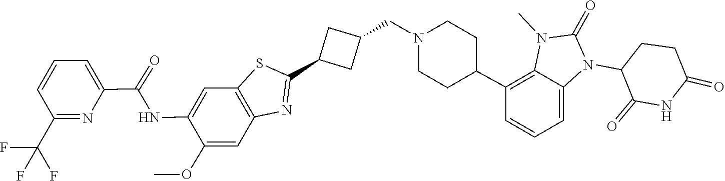 [SMILES: COc1cc2nc([C@H]3C[C@H](CN4CCC(c5cccc6c5n(C)c(=O)n6C5CCC(=O)NC5=O)CC4)C3)sc2cc1NC(=O)c1cccc(C(F)(F)F)n1] |
| I-236 | 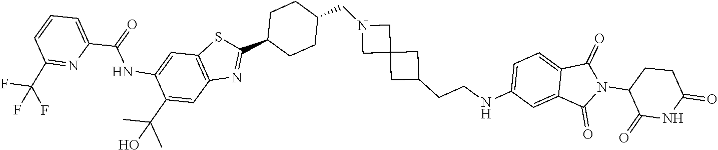 [SMILES: CC(C)(O)c1cc2nc([C@H]3CC[C@H](CN4CC5(CC(CCNc6ccc7c(c6)C(=O)N(C6CCC(=O)NC6=O)C7=O)C5)C4)CC3)sc2cc1NC(=O)c1cccc(C(F)(F)F)n1] |
| I-237 | 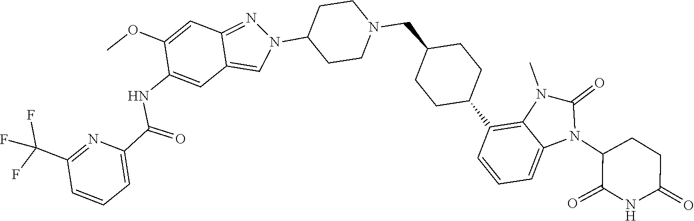 [SMILES: COc1cc2nn(C3CCN(C[C@H]4CC[C@H](c5cccc6c5n(C)c(=O)n6C5CCC(=O)NC5=O)CC4)CC3)cc2cc1NC(=O)c1cccc(C(F)(F)F)n1] |
| I-238 | 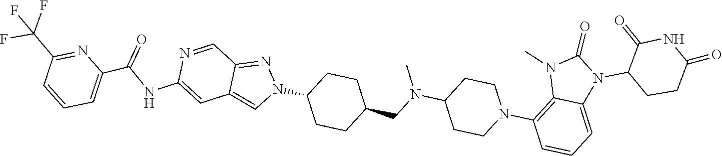 [SMILES: CN(C[C@H]1CC[C@H](n2cc3cc(NC(=O)c4cccc(C(F)(F)F)n4)ncc3n2)CC1)C1CCN(c2cccc3c2n(C)c(=O)n3C2CCC(=O)NC2=O)CC1] |
| I-239 | 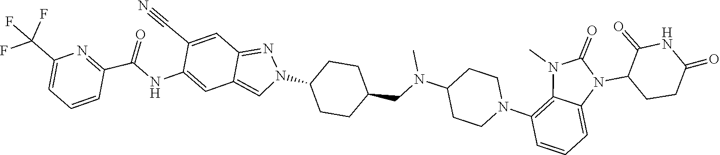 [SMILES: CN(C[C@H]1CC[C@H](n2cc3cc(NC(=O)c4cccc(C(F)(F)F)n4)c(C#N)cc3n2)CC1)C1CCN(c2cccc3c2n(C)c(=O)n3C2CCC(=O)NC2=O)CC1] |
| I-240 | 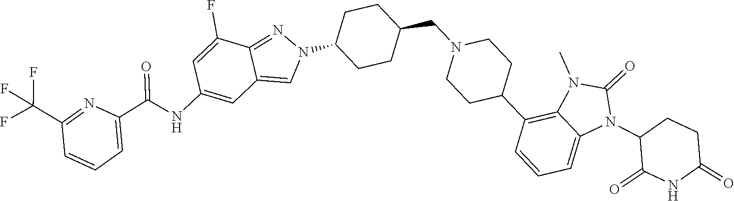 [SMILES: Cn1c(=O)n(C2CCC(=O)NC2=O)c2cccc(C3CCN(C[C@H]4CC[C@H](n5cc6cc(NC(=O)c7cccc(C(F)(F)F)n7)cc(F)c6n5)CC4)CC3)c21] |
| I-241 | 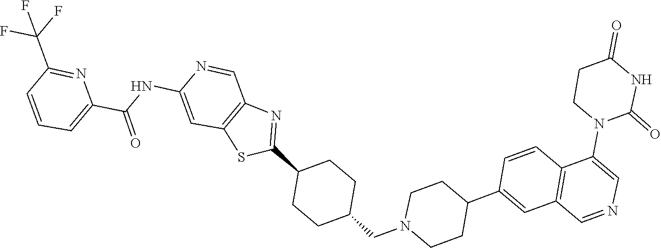 [SMILES: O=C1CCN(c2cncc3cc(C4CCN(C[C@H]5CC[C@H](c6nc7cnc(NC(=O)c8cccc(C(F)(F)F)n8)cc7s6)CC5)CC4)ccc23)C(=O)N1] |
| I-242 | 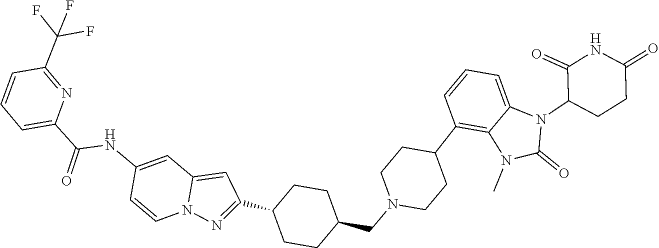 [SMILES: Cn1c(=O)n(C2CCC(=O)NC2=O)c2cccc(C3CCN(C[C@H]4CC[C@H](c5cc6cc(NC(=O)c7cccc(C(F)(F)F)n7)ccn6n5)CC4)CC3)c21] |
| I-243 | 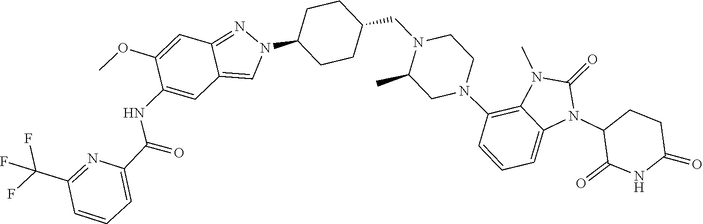 [SMILES: COc1cc2nn([C@H]3CC[C@H](CN4CCN(c5cccc6c5n(C)c(=O)n6C5CCC(=O)NC5=O)C[C@H]4C)CC3)cc2cc1NC(=O)c1cccc(C(F)(F)F)n1] |
| I-244 | 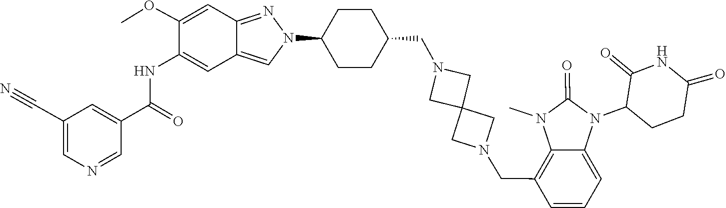 [SMILES: COc1cc2nn([C@H]3CC[C@H](CN4CC5(CN(Cc6cccc7c6n(C)c(=O)n7C6CCC(=O)NC6=O)C5)C4)CC3)cc2cc1NC(=O)c1cncc(C#N)c1] |
| I-245 | 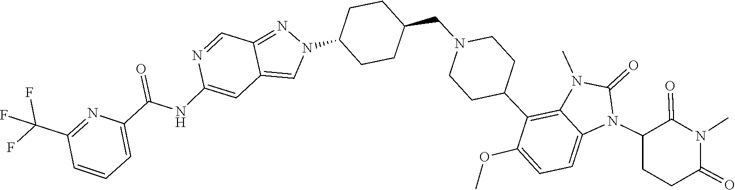 [SMILES: COc1ccc2c(c1C1CCN(C[C@H]3CC[C@H](n4cc5cc(NC(=O)c6cccc(C(F)(F)F)n6)ncc5n4)CC3)CC1)n(C)c(=O)n2C1CCC(=O)N(C)C1=O] |
| I-246 | 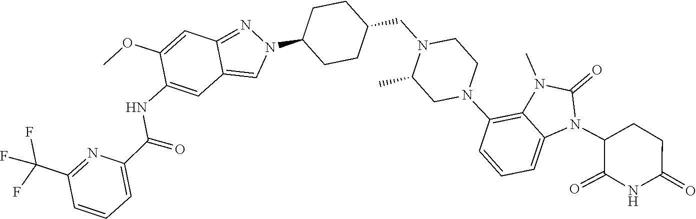 [SMILES: COc1cc2nn([C@H]3CC[C@H](CN4CCN(c5cccc6c5n(C)c(=O)n6C5CCC(=O)NC5=O)C[C@@H]4C)CC3)cc2cc1NC(=O)c1cccc(C(F)(F)F)n1] |
| I-247 | 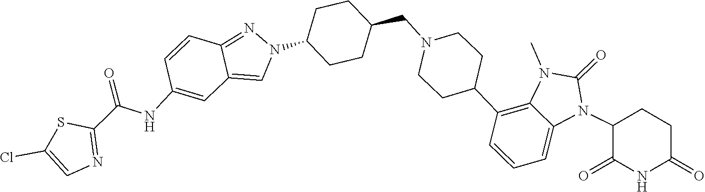 [SMILES: Cn1c(=O)n(C2CCC(=O)NC2=O)c2cccc(C3CCN(C[C@H]4CC[C@H](n5cc6cc(NC(=O)c7ncc(Cl)s7)ccc6n5)CC4)CC3)c21] |
| I-248 | 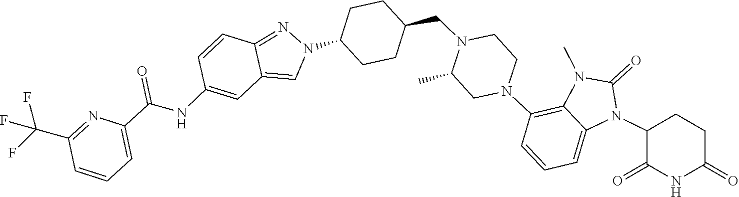 [SMILES: C[C@H]1CN(c2cccc3c2n(C)c(=O)n3C2CCC(=O)NC2=O)CCN1C[C@H]1CC[C@H](n2cc3cc(NC(=O)c4cccc(C(F)(F)F)n4)ccc3n2)CC1] |
| I-249 | 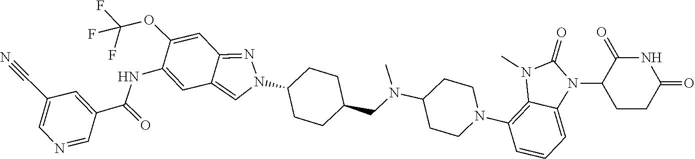 [SMILES: CN(C[C@H]1CC[C@H](n2cc3cc(NC(=O)c4cncc(C#N)c4)c(OC(F)(F)F)cc3n2)CC1)C1CCN(c2cccc3c2n(C)c(=O)n3C2CCC(=O)NC2=O)CC1] |
| I-250 | 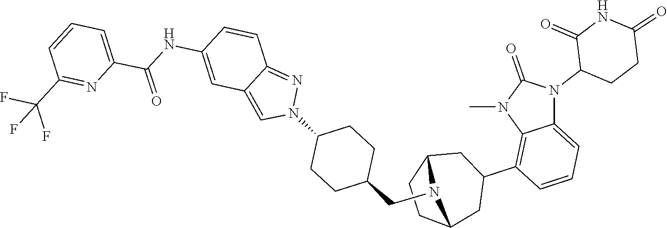 [SMILES: Cn1c(=O)n(C2CCC(=O)NC2=O)c2cccc(C3C[C@H]4CC[C@@H](C3)N4C[C@H]3CC[C@H](n4cc5cc(NC(=O)c6cccc(C(F)(F)F)n6)ccc5n4)CC3)c21] |
| I-251 | 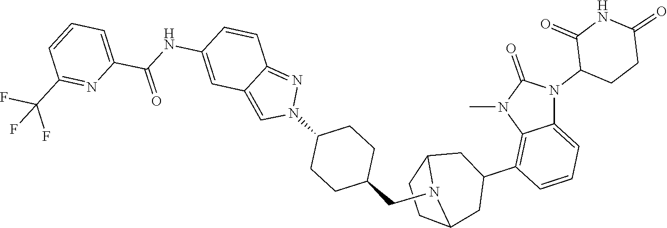 [SMILES: Cn1c(=O)n(C2CCC(=O)NC2=O)c2cccc(C3CC4CCC(C3)N4C[C@H]3CC[C@H](n4cc5cc(NC(=O)c6cccc(C(F)(F)F)n6)ccc5n4)CC3)c21] |
| I-252 | 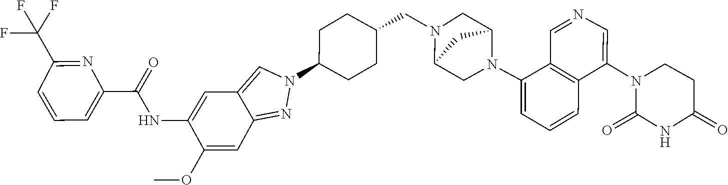 |
| I-253 | 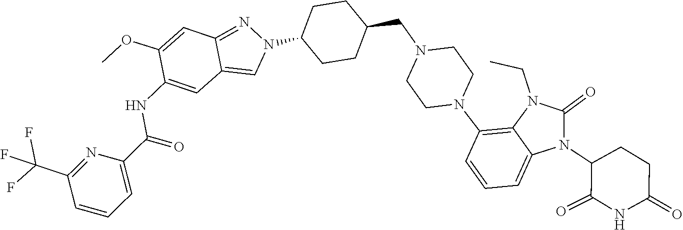 [SMILES: CCn1c(=O)n(C2CCC(=O)NC2=O)c2cccc(N3CCN(C[C@H]4CC[C@H](n5cc6cc(NC(=O)c7cccc(C(F)(F)F)n7)c(OC)cc6n5)CC4)CC3)c21] |
| I-254 | 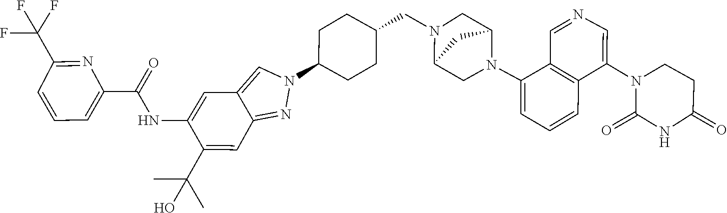 |
| I-255 | 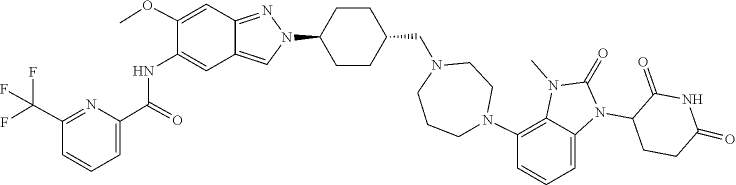 [SMILES: COc1cc2nn([C@H]3CC[C@H](CN4CCCN(c5cccc6c5n(C)c(=O)n6C5CCC(=O)NC5=O)CC4)CC3)cc2cc1NC(=O)c1cccc(C(F)(F)F)n1] |
| I-256 | 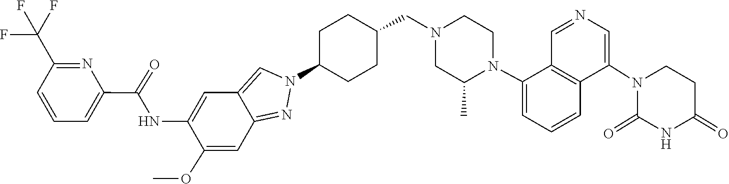 [SMILES: COc1cc2nn([C@H]3CC[C@H](CN4CCN(c5cccc6c(N7CCC(=O)NC7=O)cncc56)[C@H](C)C4)CC3)cc2cc1NC(=O)c1cccc(C(F)(F)F)n1] |
| I-257 | 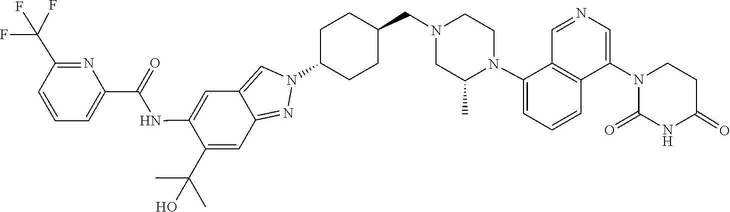 [SMILES: C[C@@H]1CN(C[C@H]2CC[C@H](n3cc4cc(NC(=O)c5cccc(C(F)(F)F)n5)c(C(C)(C)O)cc4n3)CC2)CCN1c1cccc2c(N3CCC(=O)NC3=O)cncc12] |
| I-258 | 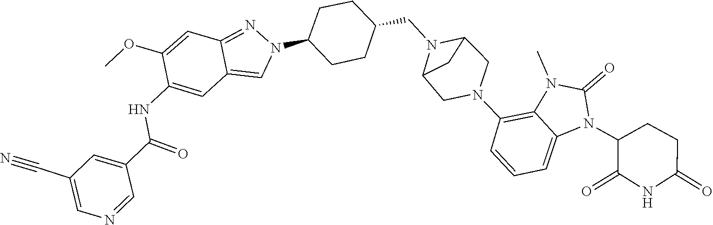 [SMILES: COc1cc2nn([C@H]3CC[C@H](CN4C5CC4CN(c4cccc6c4n(C)c(=O)n6C4CCC(=O)NC4=O)C5)CC3)cc2cc1NC(=O)c1cncc(C#N)c1] |
| I-259 | 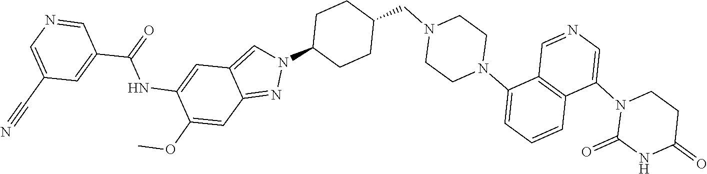 [SMILES: COc1cc2nn([C@H]3CC[C@H](CN4CCN(c5cccc6c(N7CCC(=O)NC7=O)cncc56)CC4)CC3)cc2cc1NC(=O)c1cncc(C#N)c1] |
| I-260 | 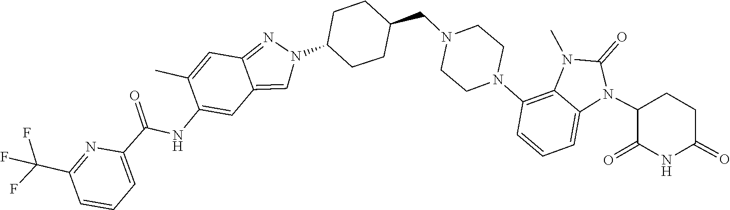 [SMILES: Cc1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-261 | 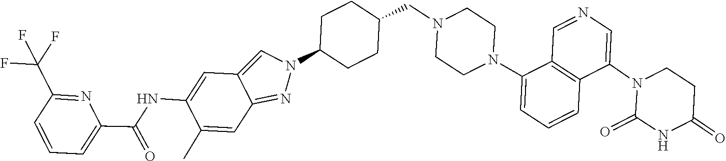 [SMILES: Cc1cc2nn([C@H]3CC[C@H](CN4CCN(c5cccc6c(N7CCC(=O)NC7=O)cncc56)CC4)CC3)cc2cc1NC(=O)c1cccc(C(F)(F)F)n1] |
| I-262 | 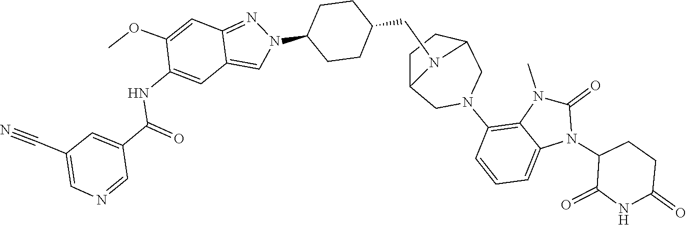 [SMILES: COc1cc2nn([C@H]3CC[C@H](CN4C5CCC4CN(c4cccc6c4n(C)c(=O)n6C4CCC(=O)NC4=O)C5)CC3)cc2cc1NC(=O)c1cncc(C#N)c1] |
| I-263 | 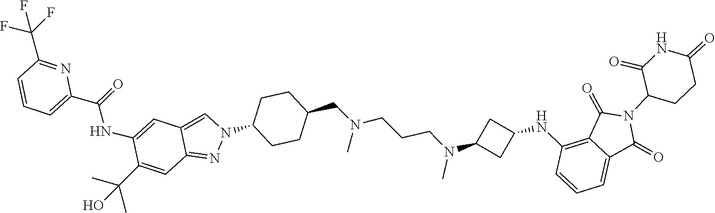 [SMILES: CN(CCCN(C)[C@H]1C[C@H](Nc2cccc3c2C(=O)N(C2CCC(=O)NC2=O)C3=O)C1)C[C@H]1CC[C@H](n2cc3cc(NC(=O)c4cccc(C(F)(F)F)n4)c(C(C)(C)O)cc3n2)CC1] |
| I-264 | 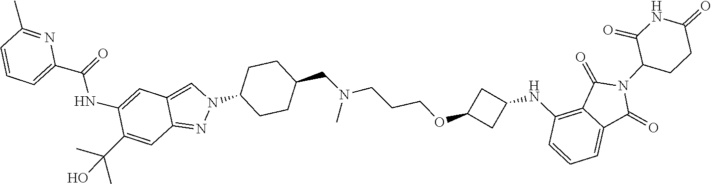 [SMILES: Cc1cccc(C(=O)Nc2cc3cn([C@H]4CC[C@H](CN(C)CCCO[C@H]5C[C@H](Nc6cccc7c6C(=O)N(C6CCC(=O)NC6=O)C7=O)C5)CC4)nc3cc2C(C)(C)O)n1] |
| I-265 | 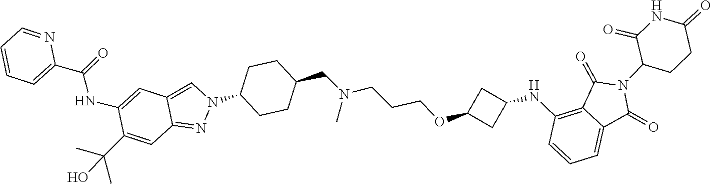 [SMILES: CN(CCCO[C@H]1C[C@H](Nc2cccc3c2C(=O)N(C2CCC(=O)NC2=O)C3=O)C1)C[C@H]1CC[C@H](n2cc3cc(NC(=O)c4ccccn4)c(C(C)(C)O)cc3n2)CC1] |
| I-266 | 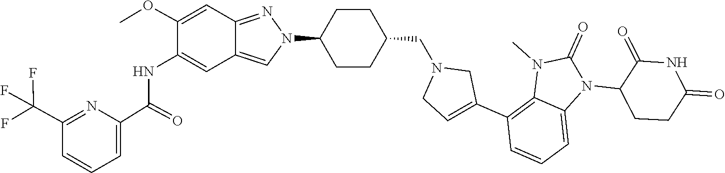 [SMILES: COc1cc2nn([C@H]3CC[C@H](CN4CC=C(c5cccc6c5n(C)c(=O)n6C5CCC(=O)NC5=O)C4)CC3)cc2cc1NC(=O)c1cccc(C(F)(F)F)n1] |
| I-267 | 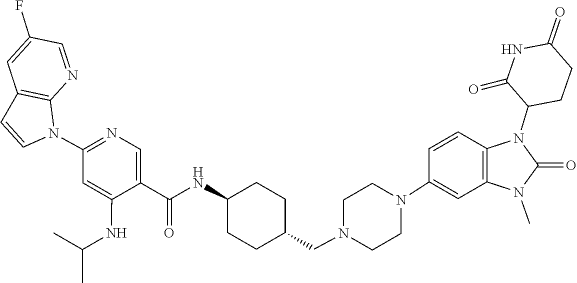 [SMILES: CC(C)Nc1cc(-n2ccc3cc(F)cnc32)ncc1C(=O)N[C@H]1CC[C@H](CN2CCN(c3ccc4c(c3)n(C)c(=O)n4C3CCC(=O)NC3=O)CC2)CC1] |
| I-268 | 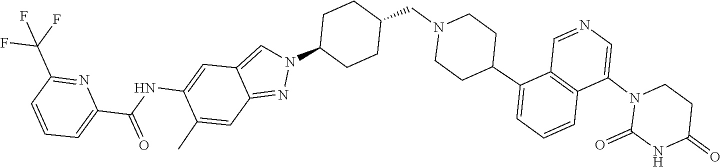 [SMILES: Cc1cc2nn([C@H]3CC[C@H](CN4CCC(c5cccc6c(N7CCC(=O)NC7=O)cncc56)CC4)CC3)cc2cc1NC(=O)c1cccc(C(F)(F)F)n1] |
| I-269 | 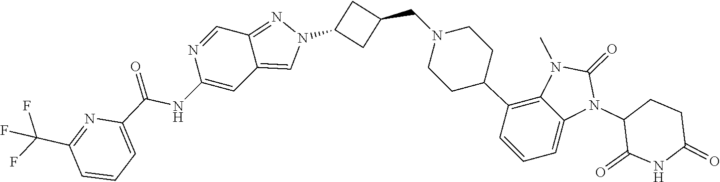 [SMILES: Cn1c(=O)n(C2CCC(=O)NC2=O)c2cccc(C3CCN(C[C@H]4C[C@H](n5cc6cc(NC(=O)c7cccc(C(F)(F)F)n7)ncc6n5)C4)CC3)c21] |
| I-270 | 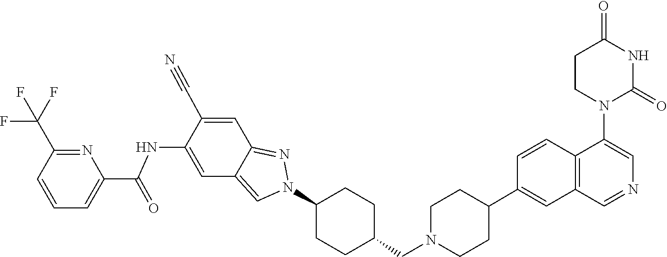 [SMILES: N#Cc1cc2nn([C@H]3CC[C@H](CN4CCC(c5ccc6c(N7CCC(=O)NC7=O)cncc6c5)CC4)CC3)cc2cc1NC(=O)c1cccc(C(F)(F)F)n1] |
| I-271 | 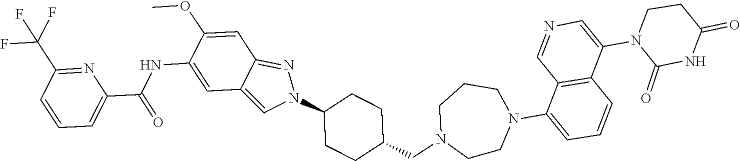 [SMILES: COc1cc2nn([C@H]3CC[C@H](CN4CCCN(c5cccc6c(N7CCC(=O)NC7=O)cncc56)CC4)CC3)cc2cc1NC(=O)c1cccc(C(F)(F)F)n1] |
| I-272 | 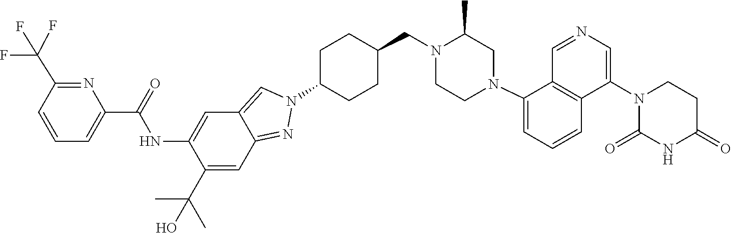 [SMILES: C[C@H]1CN(c2cccc3c(N4CCC(=O)NC4=O)cncc23)CCN1C[C@H]1CC[C@H](n2cc3cc(NC(=O)c4cccc(C(F)(F)F)n4)c(C(C)(C)O)cc3n2)CC1] |
| I-273 | 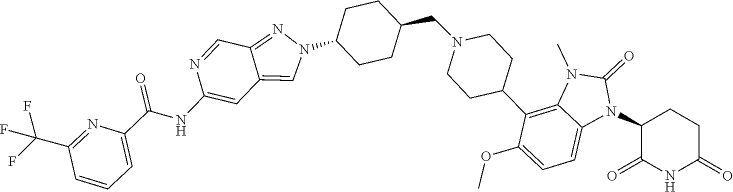 [SMILES: COc1ccc2c(c1C1CCN(C[C@H]3CC[C@H](n4cc5cc(NC(=O)c6cccc(C(F)(F)F)n6)ncc5n4)CC3)CC1)n(C)c(=O)n2[C@H]1CCC(=O)NC1=O] |
| I-274 | 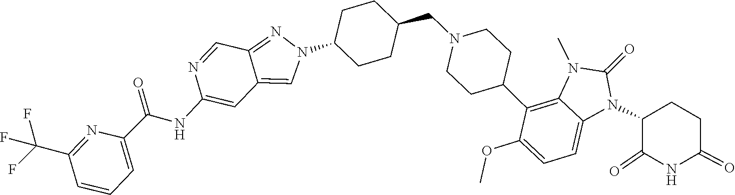 [SMILES: COc1ccc2c(c1C1CCN(C[C@H]3CC[C@H](n4cc5cc(NC(=O)c6cccc(C(F)(F)F)n6)ncc5n4)CC3)CC1)n(C)c(=O)n2[C@@H]1CCC(=O)NC1=O] |
| I-275 | 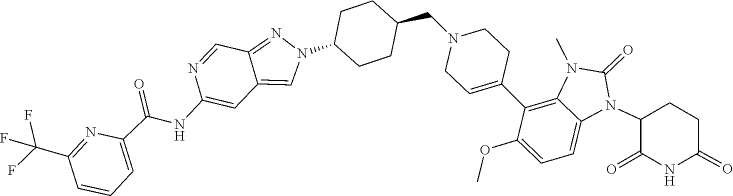 [SMILES: COc1ccc2c(c1C1=CCN(C[C@H]3CC[C@H](n4cc5cc(NC(=O)c6cccc(C(F)(F)F)n6)ncc5n4)CC3)CC1)n(C)c(=O)n2C1CCC(=O)NC1=O] |
| I-276 | 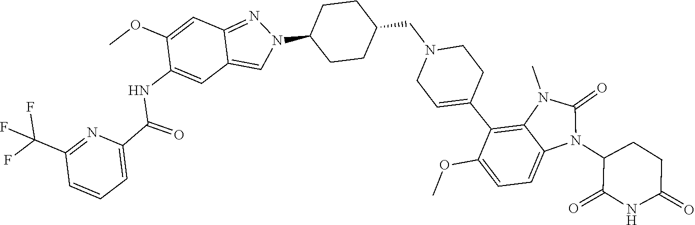 [SMILES: COc1cc2nn([C@H]3CC[C@H](CN4CC=C(c5c(OC)ccc6c5n(C)c(=O)n6C5CCC(=O)NC5=O)CC4)CC3)cc2cc1NC(=O)c1cccc(C(F)(F)F)n1] |
| I-277 | 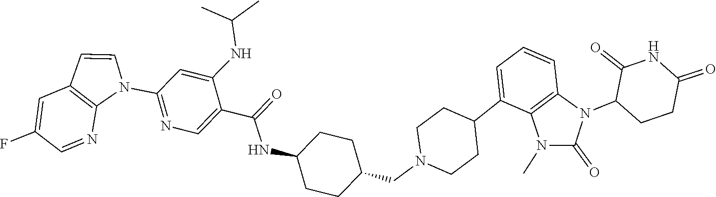 [SMILES: CC(C)Nc1cc(-n2ccc3cc(F)cnc32)ncc1C(=O)N[C@H]1CC[C@H](CN2CCC(c3cccc4c3n(C)c(=O)n4C3CCC(=O)NC3=O)CC2)CC1] |
| I-278 | 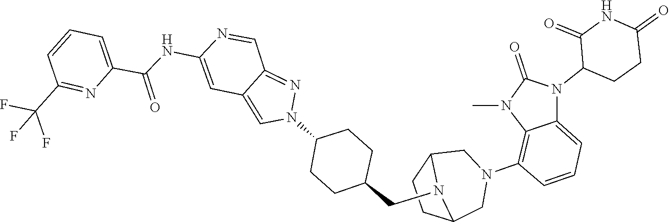 [SMILES: Cn1c(=O)n(C2CCC(=O)NC2=O)c2cccc(N3CC4CCC(C3)N4C[C@H]3CC[C@H](n4cc5cc(NC(=O)c6cccc(C(F)(F)F)n6)ncc5n4)CC3)c21] |
| I-279 | 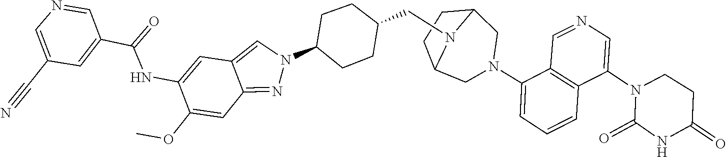 [SMILES: COc1cc2nn([C@H]3CC[C@H](CN4C5CCC4CN(c4cccc6c(N7CCC(=O)NC7=O)cncc46)C5)CC3)cc2cc1NC(=O)c1cncc(C#N)c1] |
| I-280 | 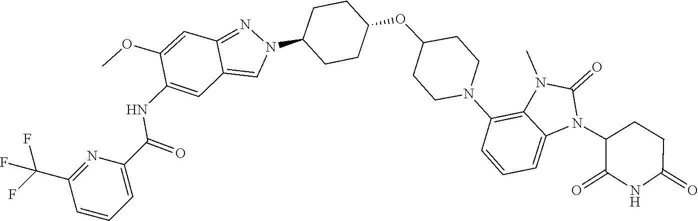 [SMILES: COc1cc2nn([C@H]3CC[C@H](OC4CCN(c5cccc6c5n(C)c(=O)n6C5CCC(=O)NC5=O)CC4)CC3)cc2cc1NC(=O)c1cccc(C(F)(F)F)n1] |
| I-281 | 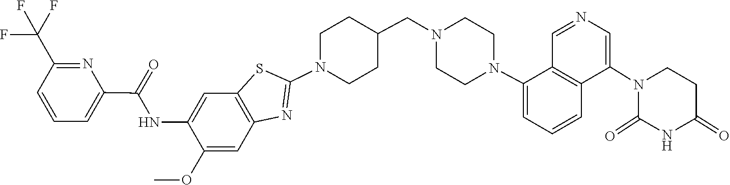 [SMILES: COc1cc2nc(N3CCC(CN4CCN(c5cccc6c(N7CCC(=O)NC7=O)cncc56)CC4)CC3)sc2cc1NC(=O)c1cccc(C(F)(F)F)n1] |
| I-282 | 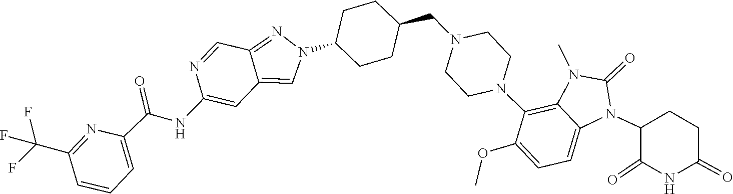 [SMILES: COc1ccc2c(c1N1CCN(C[C@H]3CC[C@H](n4cc5cc(NC(=O)c6cccc(C(F)(F)F)n6)ncc5n4)CC3)CC1)n(C)c(=O)n2C1CCC(=O)NC1=O] |
| I-283 | 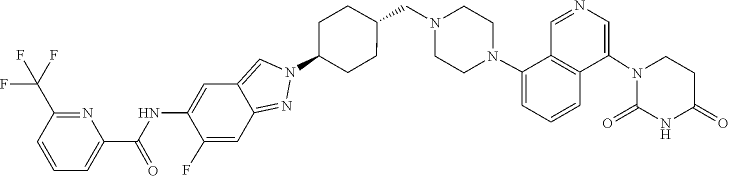 [SMILES: O=C1CCN(c2cncc3c(N4CCN(C[C@H]5CC[C@H](n6cc7cc(NC(=O)c8cccc(C(F)(F)F)n8)c(F)cc7n6)CC5)CC4)cccc23)C(=O)N1] |
| I-284 | 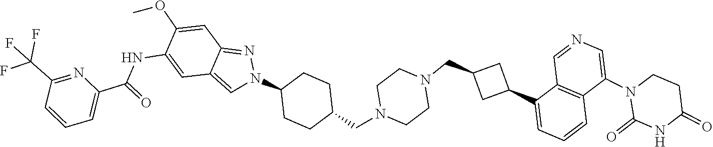 [SMILES: COc1cc2nn([C@H]3CC[C@H](CN4CCN(C[C@H]5C[C@@H](c6cccc7c(N8CCC(=O)NC8=O)cncc76)C5)CC4)CC3)cc2cc1NC(=O)c1cccc(C(F)(F)F)n1] |
| I-285 | 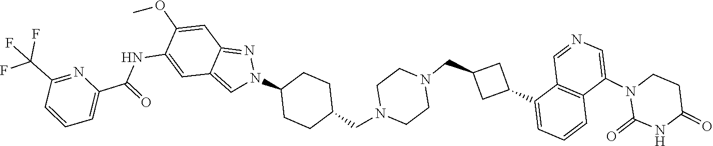 [SMILES: COc1cc2nn([C@H]3CC[C@H](CN4CCN(C[C@H]5C[C@H](c6cccc7c(N8CCC(=O)NC8=O)cncc76)C5)CC4)CC3)cc2cc1NC(=O)c1cccc(C(F)(F)F)n1] |
| I-286 | 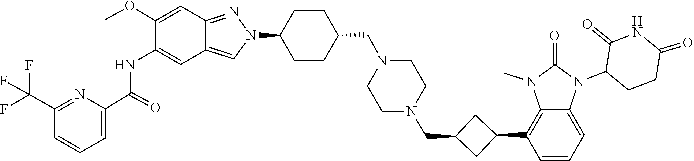 [SMILES: COc1cc2nn([C@H]3CC[C@H](CN4CCN(C[C@H]5C[C@@H](c6cccc7c6n(C)c(=O)n7C6CCC(=O)NC6=O)C5)CC4)CC3)cc2cc1NC(=O)c1cccc(C(F)(F)F)n1] |
| I-287 | 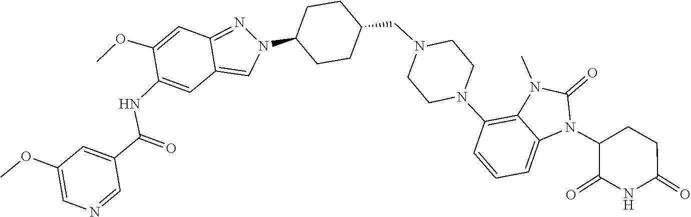 [SMILES: COc1cncc(C(=O)Nc2cc3cn([C@H]4CC[C@H](CN5CCN(c6cccc7c6n(C)c(=O)n7C6CCC(=O)NC6=O)CC5)CC4)nc3cc2OC)c1] |
| I-288 | 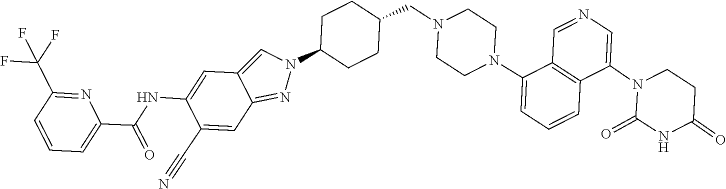 [SMILES: N#Cc1cc2nn([C@H]3CC[C@H](CN4CCN(c5cccc6c(N7CCC(=O)NC7=O)cncc56)CC4)CC3)cc2cc1NC(=O)c1cccc(C(F)(F)F)n1] |
| I-289 | 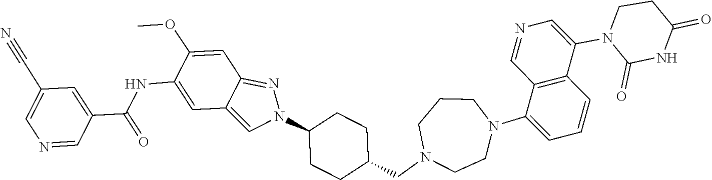 [SMILES: COc1cc2nn([C@H]3CC[C@H](CN4CCCN(c5cccc6c(N7CCC(=O)NC7=O)cncc56)CC4)CC3)cc2cc1NC(=O)c1cncc(C#N)c1] |
| I-290 | 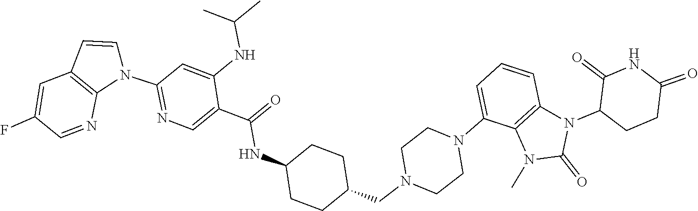 [SMILES: CC(C)Nc1cc(-n2ccc3cc(F)cnc32)ncc1C(=O)N[C@H]1CC[C@H](CN2CCN(c3cccc4c3n(C)c(=O)n4C3CCC(=O)NC3=O)CC2)CC1] |
| I-291 | 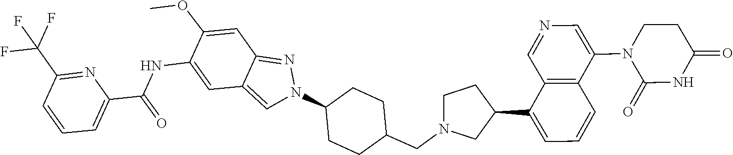 |
| I-292 | 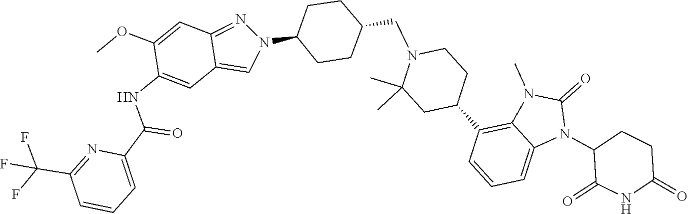 [SMILES: COc1cc2nn([C@H]3CC[C@H](CN4CC[C@H](c5cccc6c5n(C)c(=O)n6C5CCC(=O)NC5=O)CC4(C)C)CC3)cc2cc1NC(=O)c1cccc(C(F)(F)F)n1] |
| I-293 | 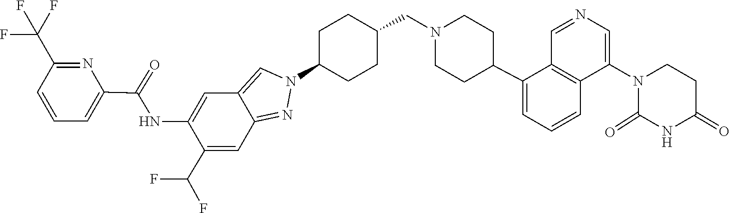 [SMILES: O=C1CCN(c2cncc3c(C4CCN(C[C@H]5CC[C@H](n6cc7cc(NC(=O)c8cccc(C(F)(F)F)n8)c(C(F)F)cc7n6)CC5)CC4)cccc23)C(=O)N1] |
| I-294 | 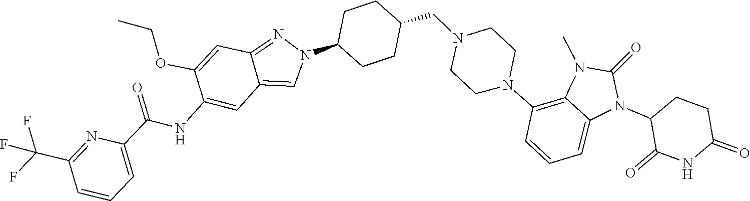 [SMILES: CCOc1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-295 | 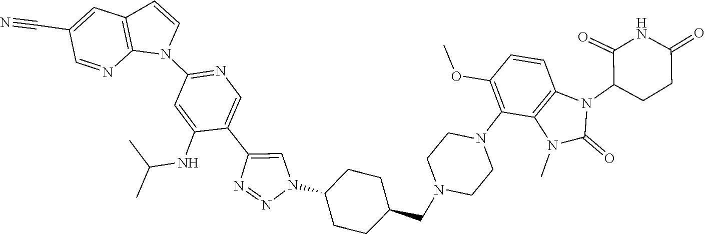 [SMILES: COc1ccc2c(c1N1CCN(C[C@H]3CC[C@H](n4cc(-c5cnc(-n6ccc7cc(C#N)cnc76)cc5NC(C)C)nn4)CC3)CC1)n(C)c(=O)n2C1CCC(=O)NC1=O] |
| I-296 | 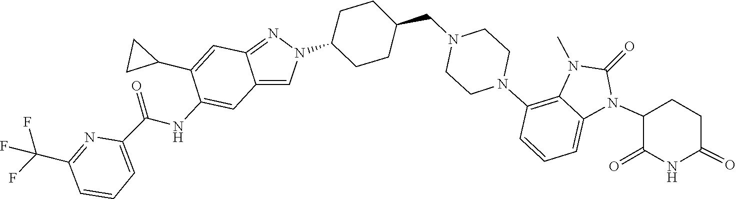 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(C7CC7)cc6n5)CC4)CC3)c21] |
| I-297 | 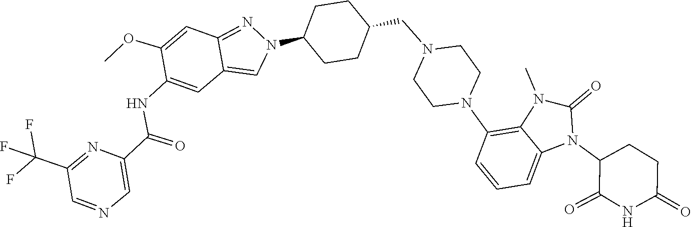 [SMILES: COc1cc2nn([C@H]3CC[C@H](CN4CCN(c5cccc6c5n(C)c(=O)n6C5CCC(=O)NC5=O)CC4)CC3)cc2cc1NC(=O)c1cncc(C(F)(F)F)n1] |
| I-298 | 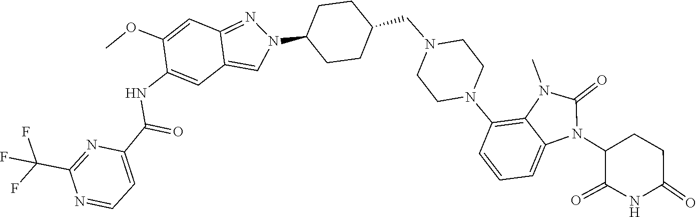 [SMILES: COc1cc2nn([C@H]3CC[C@H](CN4CCN(c5cccc6c5n(C)c(=O)n6C5CCC(=O)NC5=O)CC4)CC3)cc2cc1NC(=O)c1ccnc(C(F)(F)F)n1] |
| I-299 | 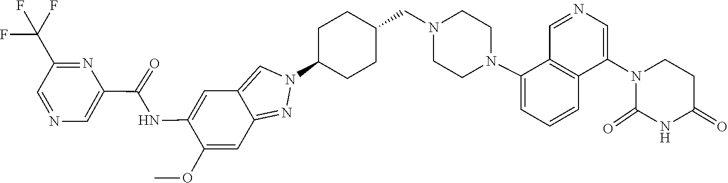 [SMILES: COc1cc2nn([C@H]3CC[C@H](CN4CCN(c5cccc6c(N7CCC(=O)NC7=O)cncc56)CC4)CC3)cc2cc1NC(=O)c1cncc(C(F)(F)F)n1] |
| I-300 | 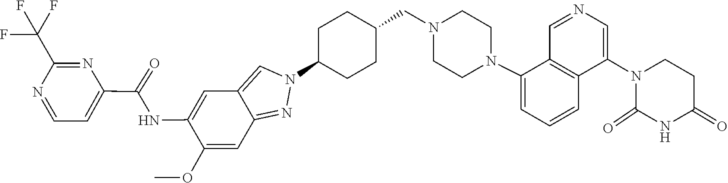 [SMILES: COc1cc2nn([C@H]3CC[C@H](CN4CCN(c5cccc6c(N7CCC(=O)NC7=O)cncc56)CC4)CC3)cc2cc1NC(=O)c1ccnc(C(F)(F)F)n1] |
| I-301 | 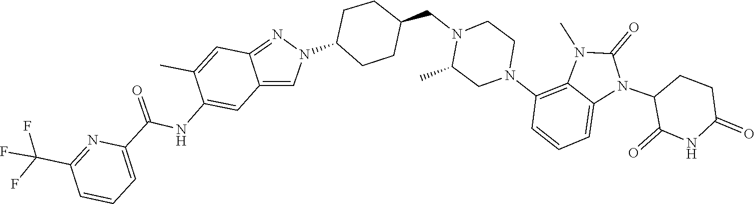 [SMILES: Cc1cc2nn([C@H]3CC[C@H](CN4CCN(c5cccc6c5n(C)c(=O)n6C5CCC(=O)NC5=O)C[C@@H]4C)CC3)cc2cc1NC(=O)c1cccc(C(F)(F)F)n1] |
| I-302 | 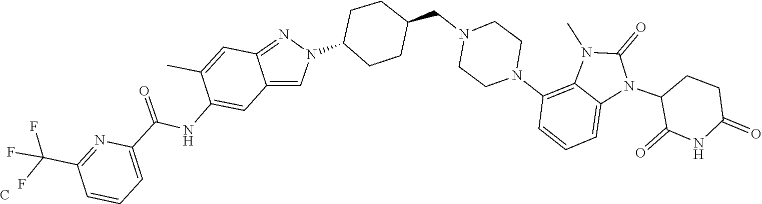 |
| I-303 | 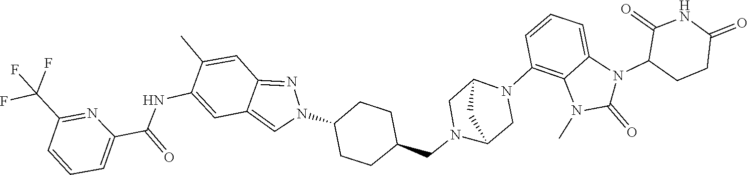 [SMILES: Cc1cc2nn([C@H]3CC[C@H](CN4C[C@@H]5C[C@H]4CN5c4cccc5c4n(C)c(=O)n5C4CCC(=O)NC4=O)CC3)cc2cc1NC(=O)c1cccc(C(F)(F)F)n1] |
| I-304 | 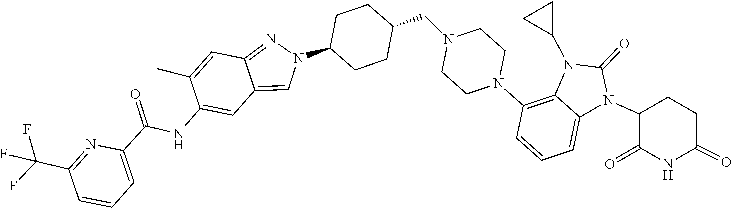 [SMILES: Cc1cc2nn([C@H]3CC[C@H](CN4CCN(c5cccc6c5n(C5CC5)c(=O)n6C5CCC(=O)NC5=O)CC4)CC3)cc2cc1NC(=O)c1cccc(C(F)(F)F)n1] |
| I-305 | 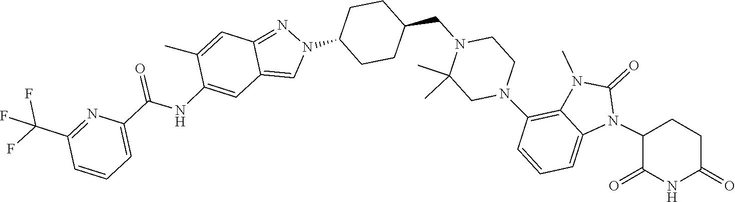 [SMILES: Cc1cc2nn([C@H]3CC[C@H](CN4CCN(c5cccc6c5n(C)c(=O)n6C5CCC(=O)NC5=O)CC4(C)C)CC3)cc2cc1NC(=O)c1cccc(C(F)(F)F)n1] |
| I-306 | 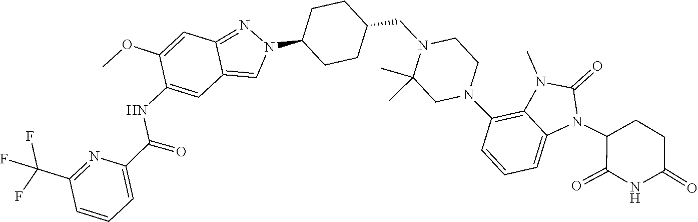 [SMILES: COc1cc2nn([C@H]3CC[C@H](CN4CCN(c5cccc6c5n(C)c(=O)n6C5CCC(=O)NC5=O)CC4(C)C)CC3)cc2cc1NC(=O)c1cccc(C(F)(F)F)n1] |
| I-307 | 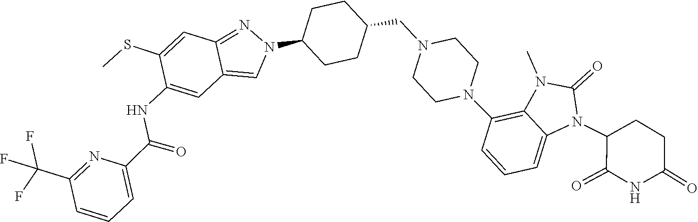 [SMILES: CSc1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-308 | 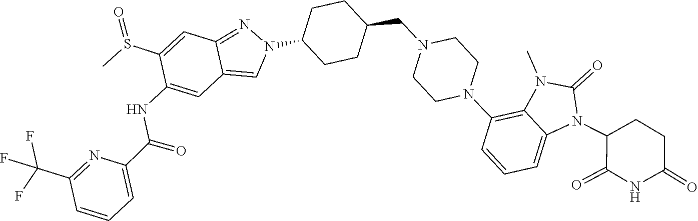 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(S(C)=O)cc6n5)CC4)CC3)c21] |
| I-309 | 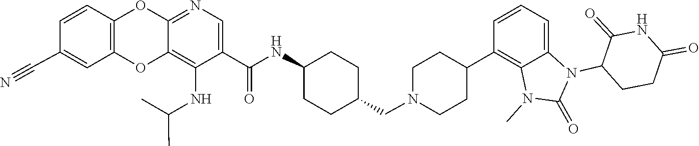 [SMILES: CC(C)Nc1c(C(=O)N[C@H]2CC[C@H](CN3CCC(c4cccc5c4n(C)c(=O)n5C4CCC(=O)NC4=O)CC3)CC2)cnc2c1Oc1cc(C#N)ccc1O2] |
| I-310 | 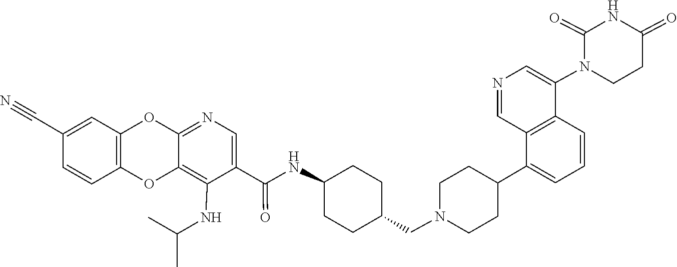 [SMILES: CC(C)Nc1c(C(=O)N[C@H]2CC[C@H](CN3CCC(c4cccc5c(N6CCC(=O)NC6=O)cncc45)CC3)CC2)cnc2c1Oc1ccc(C#N)cc1O2] |
| I-311 | 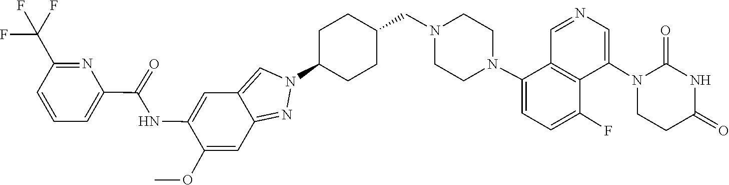 [SMILES: COc1cc2nn([C@H]3CC[C@H](CN4CCN(c5ccc(F)c6c(N7CCC(=O)NC7=O)cncc56)CC4)CC3)cc2cc1NC(=O)c1cccc(C(F)(F)F)n1] |
| I-312 | 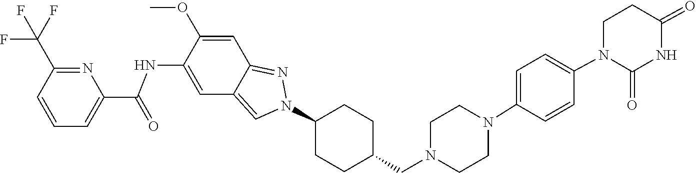 [SMILES: COc1cc2nn([C@H]3CC[C@H](CN4CCN(c5ccc(N6CCC(=O)NC6=O)cc5)CC4)CC3)cc2cc1NC(=O)c1cccc(C(F)(F)F)n1] |
| I-313 | 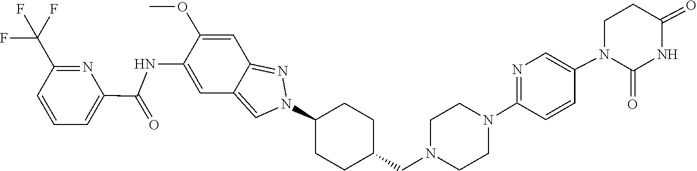 [SMILES: COc1cc2nn([C@H]3CC[C@H](CN4CCN(c5ccc(N6CCC(=O)NC6=O)cn5)CC4)CC3)cc2cc1NC(=O)c1cccc(C(F)(F)F)n1] |
| I-314 | 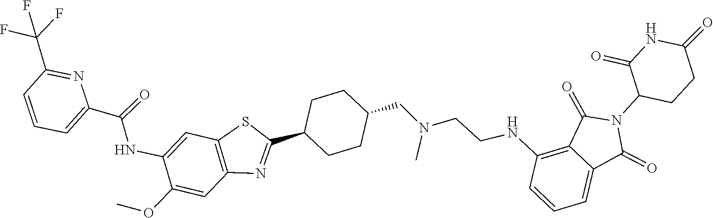 [SMILES: COc1cc2nc([C@H]3CC[C@H](CN(C)CCNc4cccc5c4C(=O)N(C4CCC(=O)NC4=O)C5=O)CC3)sc2cc1NC(=O)c1cccc(C(F)(F)F)n1] |
| I-315 | 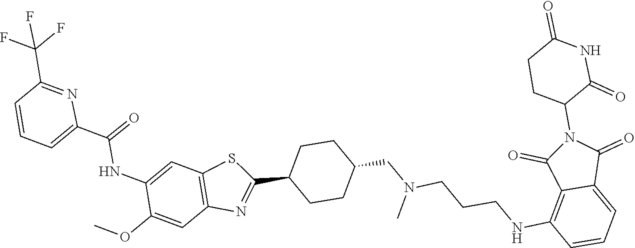 [SMILES: COc1cc2nc([C@H]3CC[C@H](CN(C)CCCNc4cccc5c4C(=O)N(C4CCC(=O)NC4=O)C5=O)CC3)sc2cc1NC(=O)c1cccc(C(F)(F)F)n1] |
| I-316 | 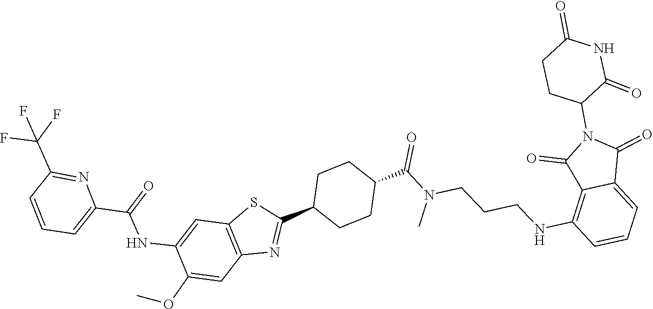 [SMILES: COc1cc2nc([C@H]3CC[C@H](C(=O)N(C)CCCNc4cccc5c4C(=O)N(C4CCC(=O)NC4=O)C5=O)CC3)sc2cc1NC(=O)c1cccc(C(F)(F)F)n1] |
| I-317 | 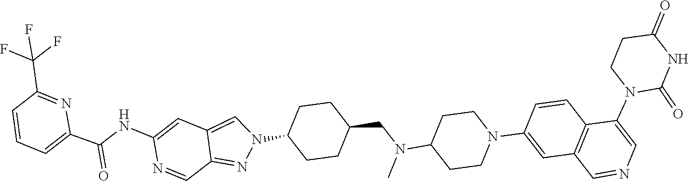 [SMILES: CN(C[C@H]1CC[C@H](n2cc3cc(NC(=O)c4cccc(C(F)(F)F)n4)ncc3n2)CC1)C1CCN(c2ccc3c(N4CCC(=O)NC4=O)cncc3c2)CC1] |
| I-318 | 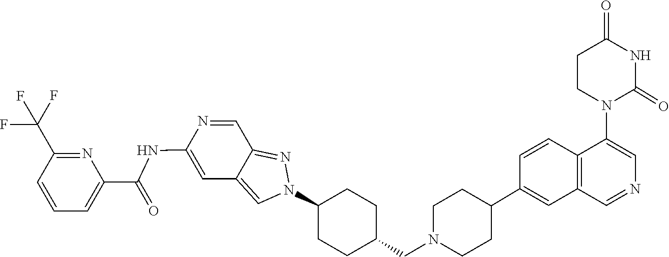 [SMILES: O=C1CCN(c2cncc3cc(C4CCN(C[C@H]5CC[C@H](n6cc7cc(NC(=O)c8cccc(C(F)(F)F)n8)ncc7n6)CC5)CC4)ccc23)C(=O)N1] |
| I-319 | 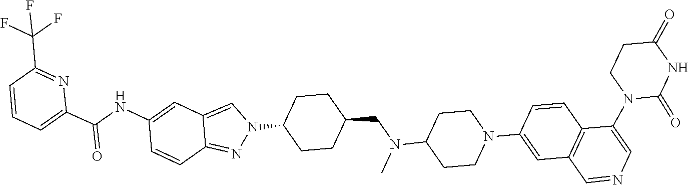 [SMILES: CN(C[C@H]1CC[C@H](n2cc3cc(NC(=O)c4cccc(C(F)(F)F)n4)ccc3n2)CC1)C1CCN(c2ccc3c(N4CCC(=O)NC4=O)cncc3c2)CC1] |
| I-320 | 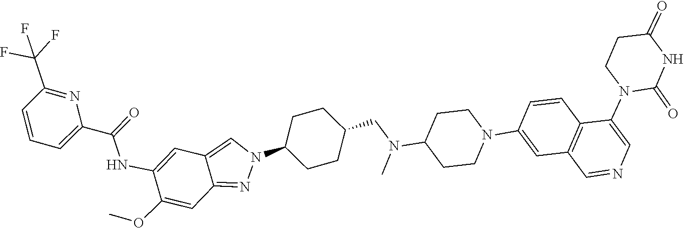 [SMILES: COc1cc2nn([C@H]3CC[C@H](CN(C)C4CCN(c5ccc6c(N7CCC(=O)NC7=O)cncc6c5)CC4)CC3)cc2cc1NC(=O)c1cccc(C(F)(F)F)n1] |
| I-321 | 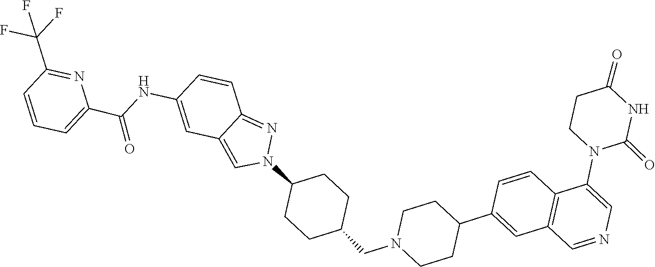 [SMILES: O=C1CCN(c2cncc3cc(C4CCN(C[C@H]5CC[C@H](n6cc7cc(NC(=O)c8cccc(C(F)(F)F)n8)ccc7n6)CC5)CC4)ccc23)C(=O)N1] |
| I-322 | 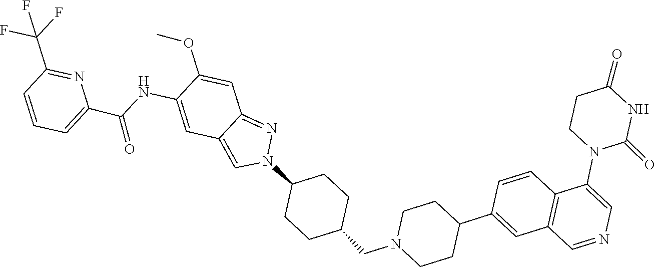 [SMILES: COc1cc2nn([C@H]3CC[C@H](CN4CCC(c5ccc6c(N7CCC(=O)NC7=O)cncc6c5)CC4)CC3)cc2cc1NC(=O)c1cccc(C(F)(F)F)n1] |
| I-323 | 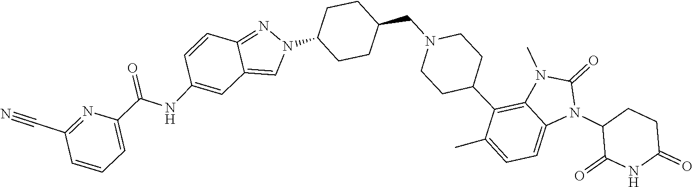 [SMILES: Cc1ccc2c(c1C1CCN(C[C@H]3CC[C@H](n4cc5cc(NC(=O)c6cccc(C#N)n6)ccc5n4)CC3)CC1)n(C)c(=O)n2C1CCC(=O)NC1=O] |
| I-324 | 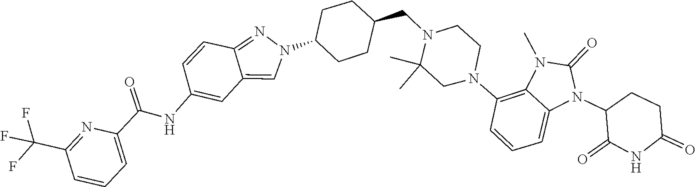 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)ccc6n5)CC4)C(C)(C)C3)c21] |
| I-325 | 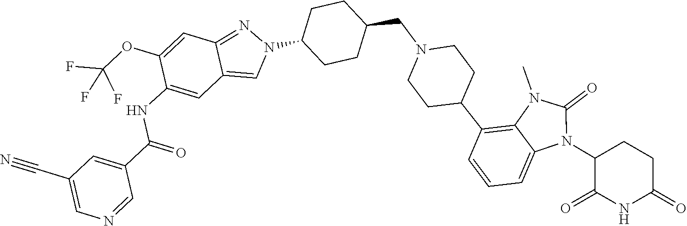 [SMILES: Cn1c(=O)n(C2CCC(=O)NC2=O)c2cccc(C3CCN(C[C@H]4CC[C@H](n5cc6cc(NC(=O)c7cncc(C#N)c7)c(OC(F)(F)F)cc6n5)CC4)CC3)c21] |
| I-326 | 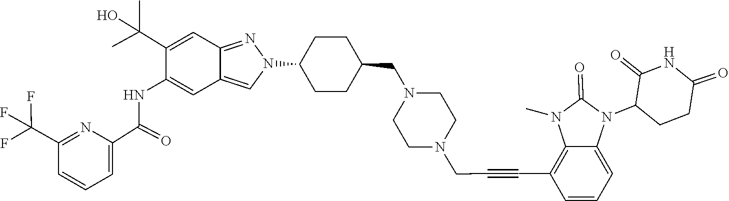 [SMILES: Cn1c(=O)n(C2CCC(=O)NC2=O)c2cccc(C#CCN3CCN(C[C@H]4CC[C@H](n5cc6cc(NC(=O)c7cccc(C(F)(F)F)n7)c(C(C)(C)O)cc6n5)CC4)CC3)c21] |
| I-327 | 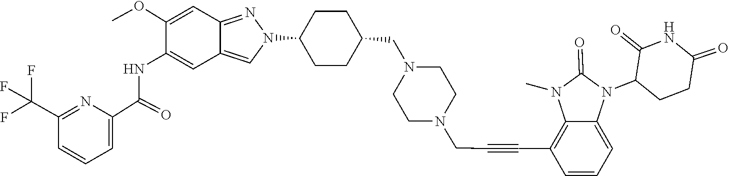 [SMILES: COc1cc2nn([C@H]3CC[C@@H](CN4CCN(CC#Cc5cccc6c5n(C)c(=O)n6C5CCC(=O)NC5=O)CC4)CC3)cc2cc1NC(=O)c1cccc(C(F)(F)F)n1] |
| I-328 | 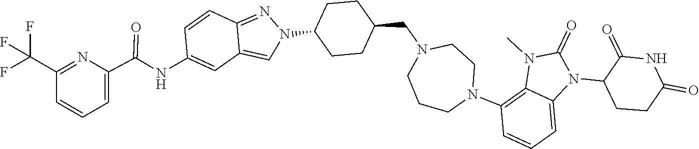 [SMILES: Cn1c(=O)n(C2CCC(=O)NC2=O)c2cccc(N3CCCN(C[C@H]4CC[C@H](n5cc6cc(NC(=O)c7cccc(C(F)(F)F)n7)ccc6n5)CC4)CC3)c21] |
| I-329 | 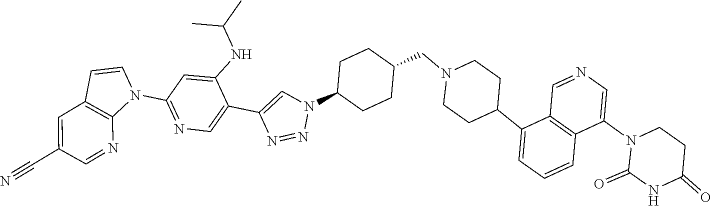 [SMILES: CC(C)Nc1cc(-n2ccc3cc(C#N)cnc32)ncc1-c1cn([C@H]2CC[C@H](CN3CCC(c4cccc5c(N6CCC(=O)NC6=O)cncc45)CC3)CC2)nn1] |
| I-330 | 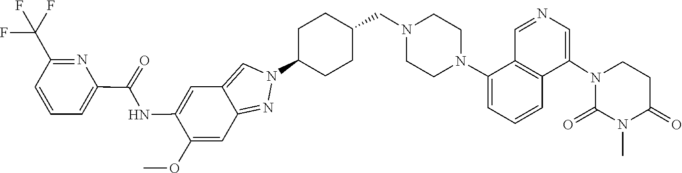 [SMILES: COc1cc2nn([C@H]3CC[C@H](CN4CCN(c5cccc6c(N7CCC(=O)N(C)C7=O)cncc56)CC4)CC3)cc2cc1NC(=O)c1cccc(C(F)(F)F)n1] |
| I-331 | 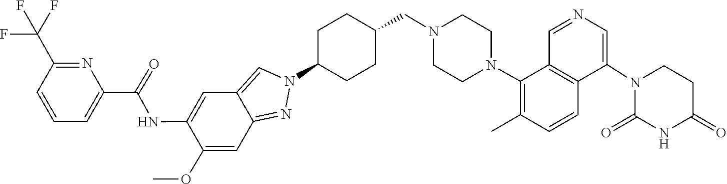 [SMILES: COc1cc2nn([C@H]3CC[C@H](CN4CCN(c5c(C)ccc6c(N7CCC(=O)NC7=O)cncc56)CC4)CC3)cc2cc1NC(=O)c1cccc(C(F)(F)F)n1] |
| I-332 | 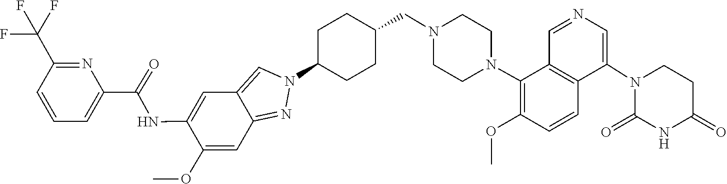 [SMILES: COc1cc2nn([C@H]3CC[C@H](CN4CCN(c5c(OC)ccc6c(N7CCC(=O)NC7=O)cncc56)CC4)CC3)cc2cc1NC(=O)c1cccc(C(F)(F)F)n1] |
| I-333 | 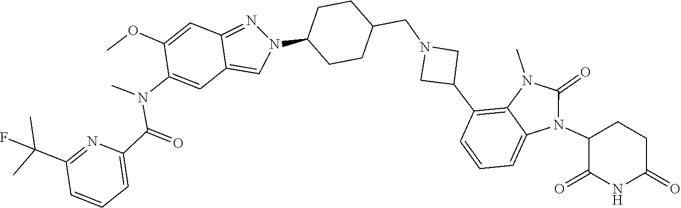 |
| I-334 | 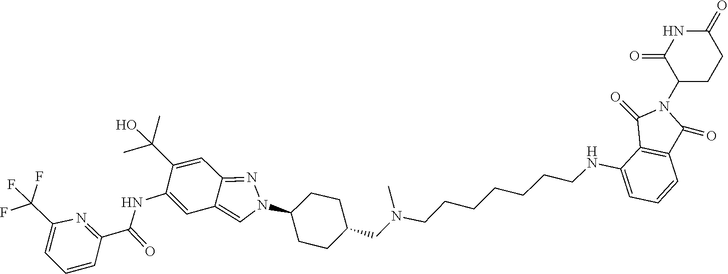 [SMILES: CN(CCCCCCCNc1cccc2c1C(=O)N(C1CCC(=O)NC1=O)C2=O)C[C@H]1CC[C@H](n2cc3cc(NC(=O)c4cccc(C(F)(F)F)n4)c(C(C)(C)O)cc3n2)CC1] |
| I-335 | 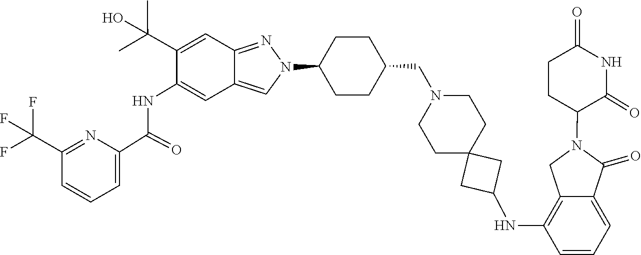 [SMILES: CC(C)(O)c1cc2nn([C@H]3CC[C@H](CN4CCC5(CC4)CC(Nc4cccc6c4CN(C4CCC(=O)NC4=O)C6=O)C5)CC3)cc2cc1NC(=O)c1cccc(C(F)(F)F)n1] |
| I-336 | 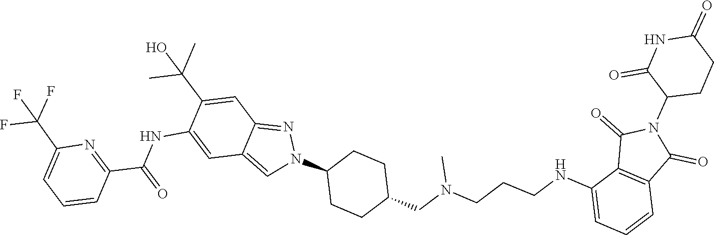 [SMILES: CN(CCCNc1cccc2c1C(=O)N(C1CCC(=O)NC1=O)C2=O)C[C@H]1CC[C@H](n2cc3cc(NC(=O)c4cccc(C(F)(F)F)n4)c(C(C)(C)O)cc3n2)CC1] |
| I-337 | 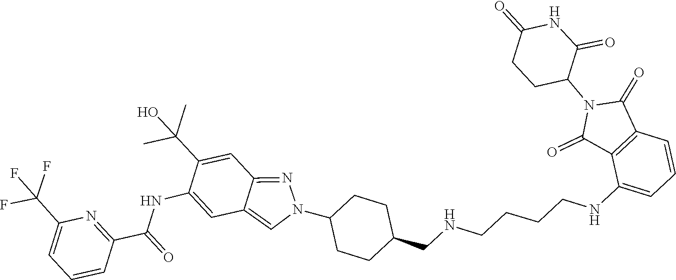 |
| I-338 | 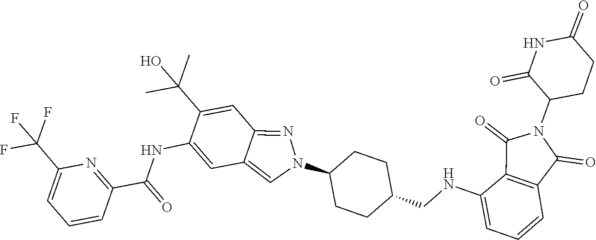 [SMILES: CC(C)(O)c1cc2nn([C@H]3CC[C@H](CNc4cccc5c4C(=O)N(C4CCC(=O)NC4=O)C5=O)CC3)cc2cc1NC(=O)c1cccc(C(F)(F)F)n1] |
| I-339 | 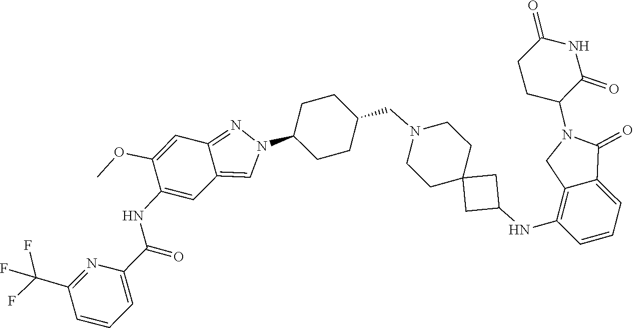 [SMILES: COc1cc2nn([C@H]3CC[C@H](CN4CCC5(CC4)CC(Nc4cccc6c4CN(C4CCC(=O)NC4=O)C6=O)C5)CC3)cc2cc1NC(=O)c1cccc(C(F)(F)F)n1] |
| I-340 | 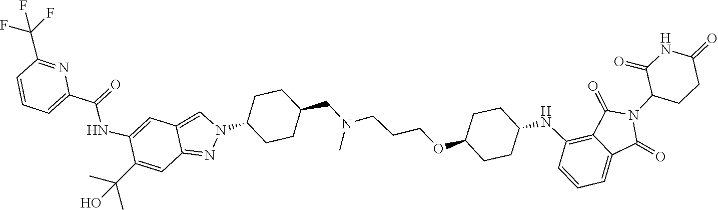 [SMILES: CN(CCCO[C@H]1CC[C@H](Nc2cccc3c2C(=O)N(C2CCC(=O)NC2=O)C3=O)CC1)C[C@H]1CC[C@H](n2cc3cc(NC(=O)c4cccc(C(F)(F)F)n4)c(C(C)(C)O)cc3n2)CC1] |
| I-341 | 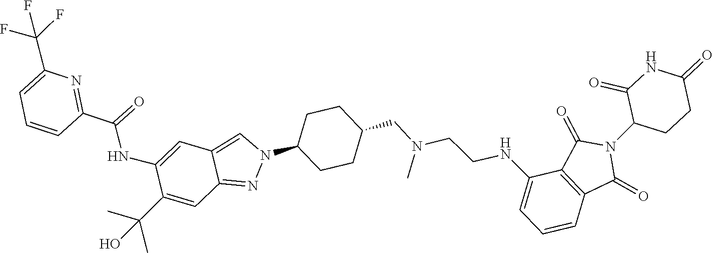 [SMILES: CN(CCNc1cccc2c1C(=O)N(C1CCC(=O)NC1=O)C2=O)C[C@H]1CC[C@H](n2cc3cc(NC(=O)c4cccc(C(F)(F)F)n4)c(C(C)(C)O)cc3n2)CC1] |
| I-342 | 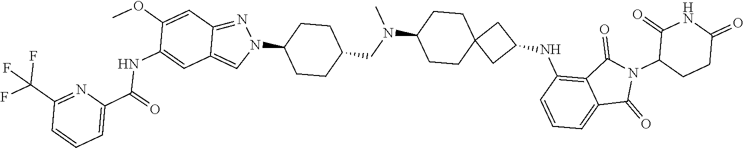 |
| I-343 | 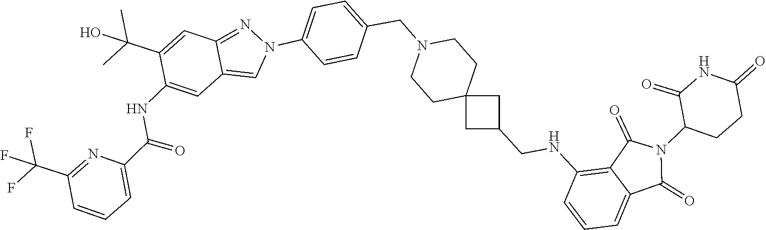 [SMILES: CC(C)(O)c1cc2nn(-c3ccc(CN4CCC5(CC4)CC(CNc4cccc6c4C(=O)N(C4CCC(=O)NC4=O)C6=O)C5)cc3)cc2cc1NC(=O)c1cccc(C(F)(F)F)n1] |
| I-344 | 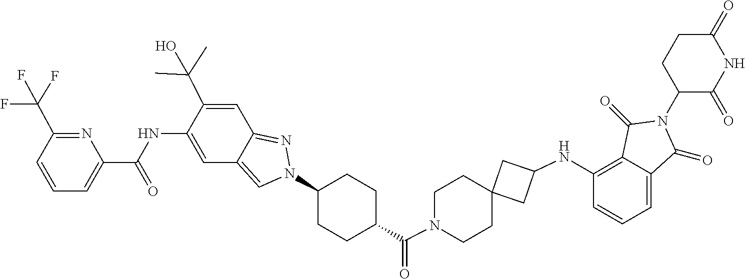 [SMILES: CC(C)(O)c1cc2nn([C@H]3CC[C@H](C(=O)N4CCC5(CC4)CC(Nc4cccc6c4C(=O)N(C4CCC(=O)NC4=O)C6=O)C5)CC3)cc2cc1NC(=O)c1cccc(C(F)(F)F)n1] |
| I-345 | 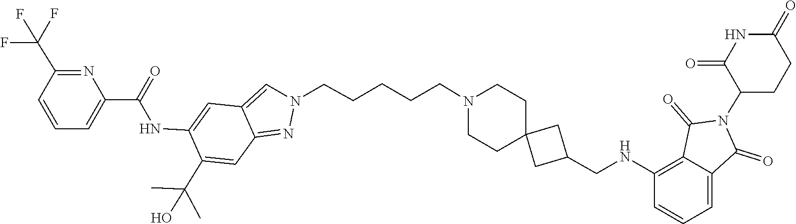 [SMILES: CC(C)(O)c1cc2nn(CCCCCN3CCC4(CC3)CC(CNc3cccc5c3C(=O)N(C3CCC(=O)NC3=O)C5=O)C4)cc2cc1NC(=O)c1cccc(C(F)(F)F)n1] |
| I-346 | 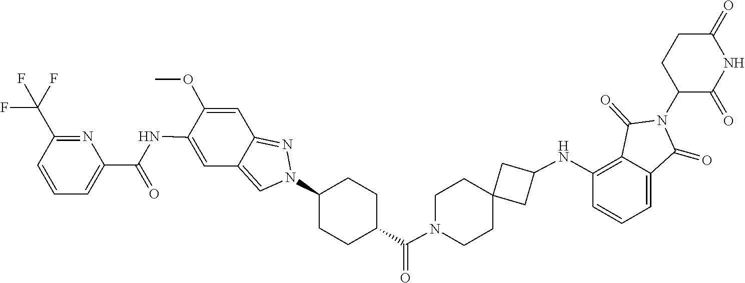 [SMILES: COc1cc2nn([C@H]3CC[C@H](C(=O)N4CCC5(CC4)CC(Nc4cccc6c4C(=O)N(C4CCC(=O)NC4=O)C6=O)C5)CC3)cc2cc1NC(=O)c1cccc(C(F)(F)F)n1] |
| I-347 | 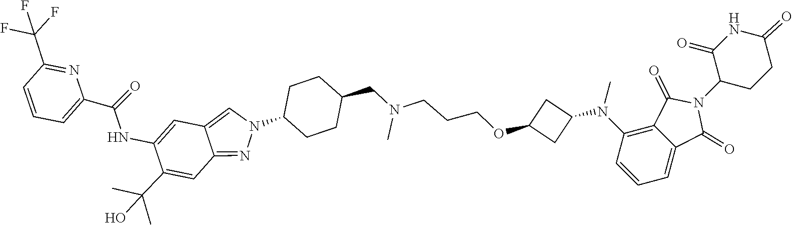 [SMILES: CN(CCCO[C@H]1C[C@H](N(C)c2cccc3c2C(=O)N(C2CCC(=O)NC2=O)C3=O)C1)C[C@H]1CC[C@H](n2cc3cc(NC(=O)c4cccc(C(F)(F)F)n4)c(C(C)(C)O)cc3n2)CC1] |
| I-348 | 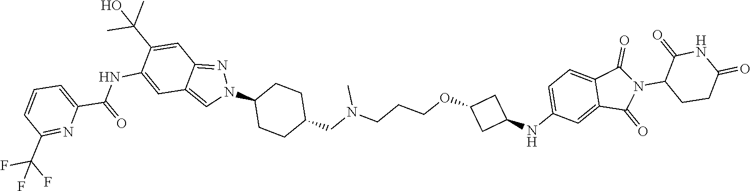 [SMILES: CN(CCCO[C@H]1C[C@H](Nc2ccc3c(c2)C(=O)N(C2CCC(=O)NC2=O)C3=O)C1)C[C@H]1CC[C@H](n2cc3cc(NC(=O)c4cccc(C(F)(F)F)n4)c(C(C)(C)O)cc3n2)CC1] |
| I-349 | 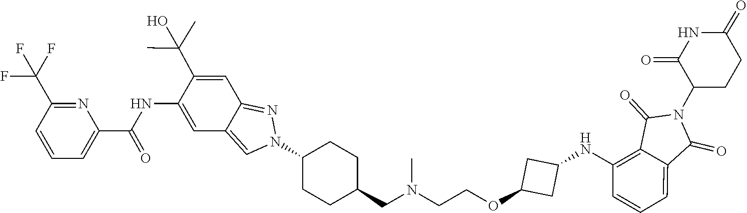 [SMILES: CN(CCO[C@H]1C[C@H](Nc2cccc3c2C(=O)N(C2CCC(=O)NC2=O)C3=O)C1)C[C@H]1CC[C@H](n2cc3cc(NC(=O)c4cccc(C(F)(F)F)n4)c(C(C)(C)O)cc3n2)CC1] |
| I-350 | 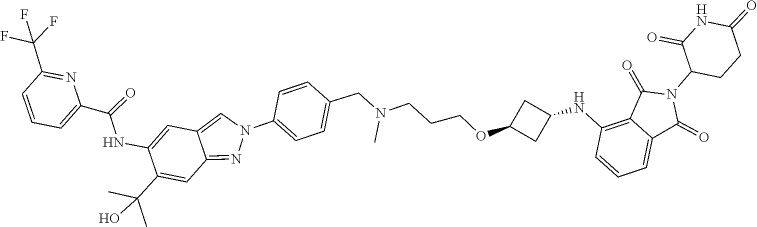 [SMILES: CN(CCCO[C@H]1C[C@H](Nc2cccc3c2C(=O)N(C2CCC(=O)NC2=O)C3=O)C1)Cc1ccc(-n2cc3cc(NC(=O)c4cccc(C(F)(F)F)n4)c(C(C)(C)O)cc3n2)cc1] |
| I-351 | 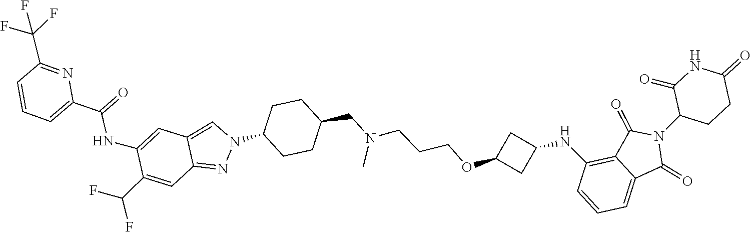 [SMILES: CN(CCCO[C@H]1C[C@H](Nc2cccc3c2C(=O)N(C2CCC(=O)NC2=O)C3=O)C1)C[C@H]1CC[C@H](n2cc3cc(NC(=O)c4cccc(C(F)(F)F)n4)c(C(F)F)cc3n2)CC1] |
| I-352 | 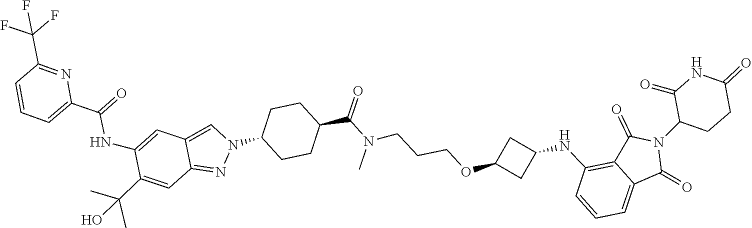 [SMILES: CN(CCCO[C@H]1C[C@H](Nc2cccc3c2C(=O)N(C2CCC(=O)NC2=O)C3=O)C1)C(=O)[C@H]1CC[C@H](n2cc3cc(NC(=O)c4cccc(C(F)(F)F)n4)c(C(C)(C)O)cc3n2)CC1] |
| I-353 | 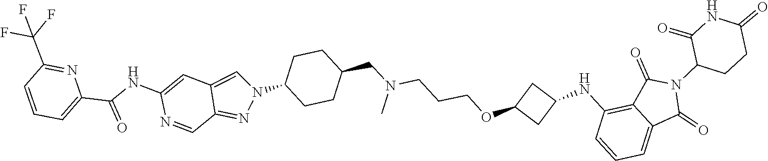 [SMILES: CN(CCCO[C@H]1C[C@H](Nc2cccc3c2C(=O)N(C2CCC(=O)NC2=O)C3=O)C1)C[C@H]1CC[C@H](n2cc3cc(NC(=O)c4cccc(C(F)(F)F)n4)ncc3n2)CC1] |
| I-354 | 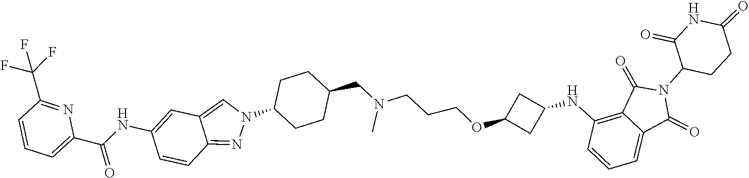 [SMILES: CN(CCCO[C@H]1C[C@H](Nc2cccc3c2C(=O)N(C2CCC(=O)NC2=O)C3=O)C1)C[C@H]1CC[C@H](n2cc3cc(NC(=O)c4cccc(C(F)(F)F)n4)ccc3n2)CC1] |
| I-355 | 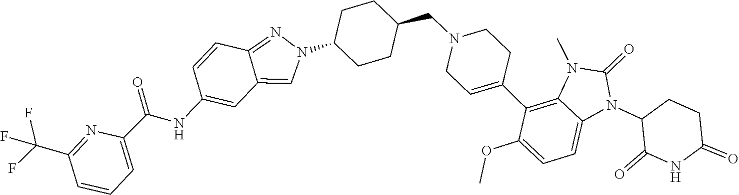 [SMILES: COc1ccc2c(c1C1=CCN(C[C@H]3CC[C@H](n4cc5cc(NC(=O)c6cccc(C(F)(F)F)n6)ccc5n4)CC3)CC1)n(C)c(=O)n2C1CCC(=O)NC1=O] |
| I-356 | 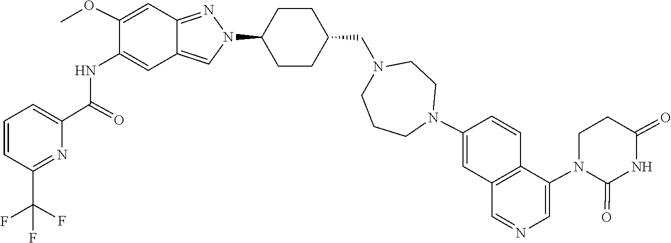 [SMILES: COc1cc2nn([C@H]3CC[C@H](CN4CCCN(c5ccc6c(N7CCC(=O)NC7=O)cncc6c5)CC4)CC3)cc2cc1NC(=O)c1cccc(C(F)(F)F)n1] |
| I-357 | 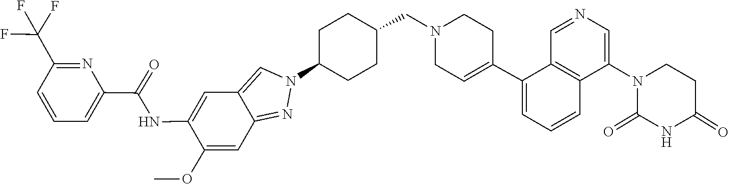 [SMILES: COc1cc2nn([C@H]3CC[C@H](CN4CC=C(c5cccc6c(N7CCC(=O)NC7=O)cncc56)CC4)CC3)cc2cc1NC(=O)c1cccc(C(F)(F)F)n1] |
| I-358 | 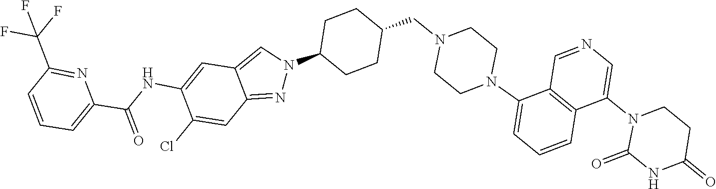 [SMILES: O=C1CCN(c2cncc3c(N4CCN(C[C@H]5CC[C@H](n6cc7cc(NC(=O)c8cccc(C(F)(F)F)n8)c(Cl)cc7n6)CC5)CC4)cccc23)C(=O)N1] |
| I-359 | 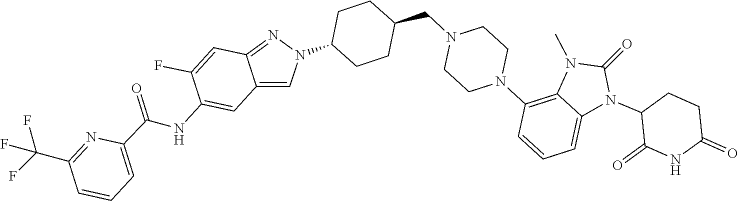 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(F)cc6n5)CC4)CC3)c21] |
| I-360 | 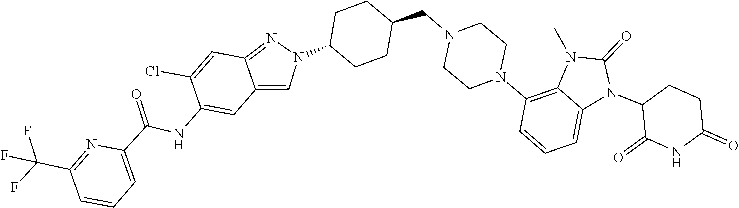 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(Cl)cc6n5)CC4)CC3)c21] |
| I-361 | 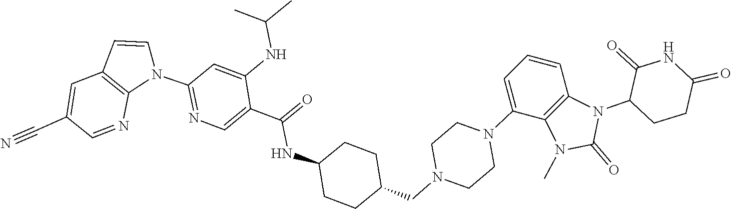 [SMILES: CC(C)Nc1cc(-n2ccc3cc(C#N)cnc32)ncc1C(=O)N[C@H]1CC[C@H](CN2CCN(c3cccc4c3n(C)c(=O)n4C3CCC(=O)NC3=O)CC2)CC1] |
| I-362 | 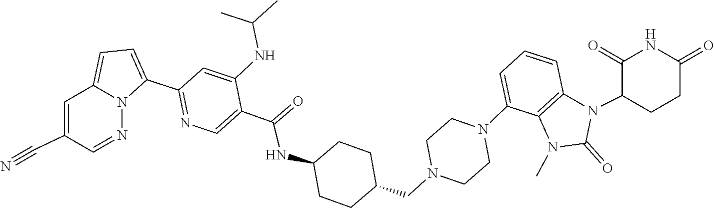 [SMILES: CC(C)Nc1cc(-c2ccc3cc(C#N)cnn23)ncc1C(=O)N[C@H]1CC[C@H](CN2CCN(c3cccc4c3n(C)c(=O)n4C3CCC(=O)NC3=O)CC2)CC1] |
| I-363 | 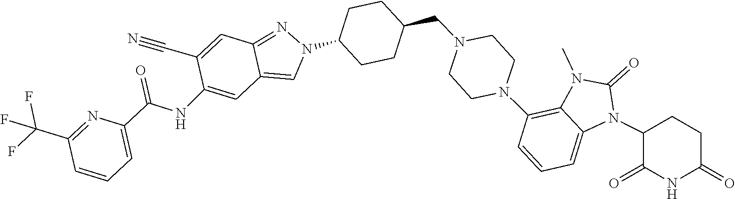 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(C#N)cc6n5)CC4)CC3)c21] |
| I-364 | 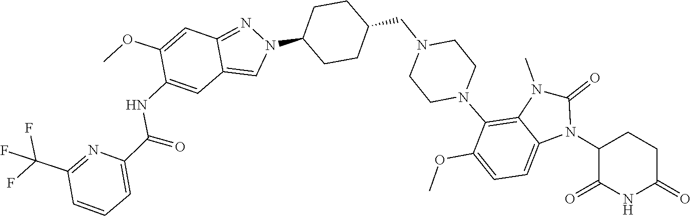 [SMILES: COc1cc2nn([C@H]3CC[C@H](CN4CCN(c5c(OC)ccc6c5n(C)c(=O)n6C5CCC(=O)NC5=O)CC4)CC3)cc2cc1NC(=O)c1cccc(C(F)(F)F)n1] |
| I-365 | 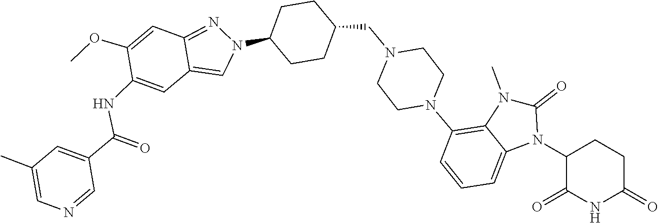 [SMILES: COc1cc2nn([C@H]3CC[C@H](CN4CCN(c5cccc6c5n(C)c(=O)n6C5CCC(=O)NC5=O)CC4)CC3)cc2cc1NC(=O)c1cncc(C)c1] |
| I-366 | 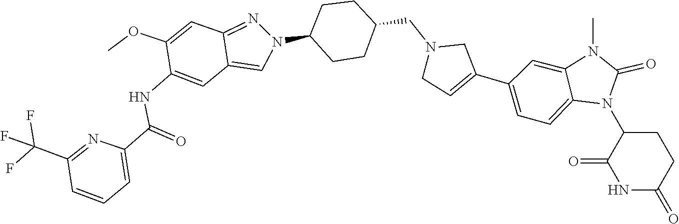 [SMILES: COc1cc2nn([C@H]3CC[C@H](CN4CC=C(c5ccc6c(c5)n(C)c(=O)n6C5CCC(=O)NC5=O)C4)CC3)cc2cc1NC(=O)c1cccc(C(F)(F)F)n1] |
| I-367 | 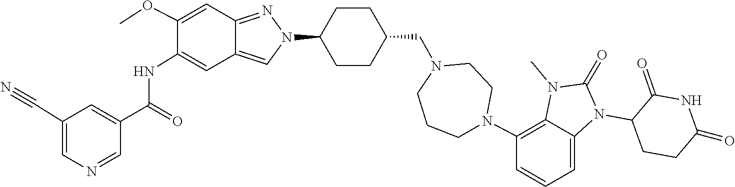 [SMILES: COc1cc2nn([C@H]3CC[C@H](CN4CCCN(c5cccc6c5n(C)c(=O)n6C5CCC(=O)NC5=O)CC4)CC3)cc2cc1NC(=O)c1cncc(C#N)c1] |
| I-368 | 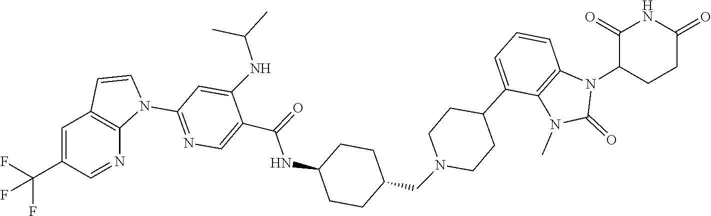 [SMILES: CC(C)Nc1cc(-n2ccc3cc(C(F)(F)F)cnc32)ncc1C(=O)N[C@H]1CC[C@H](CN2CCC(c3cccc4c3n(C)c(=O)n4C3CCC(=O)NC3=O)CC2)CC1] |
| I-369 | 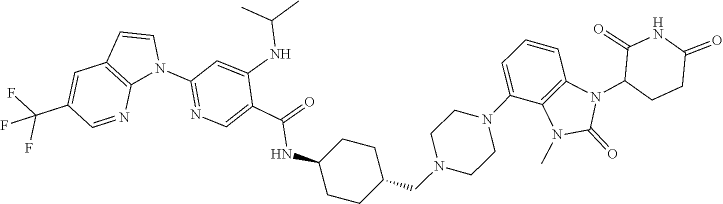 [SMILES: CC(C)Nc1cc(-n2ccc3cc(C(F)(F)F)cnc32)ncc1C(=O)N[C@H]1CC[C@H](CN2CCN(c3cccc4c3n(C)c(=O)n4C3CCC(=O)NC3=O)CC2)CC1] |
| I-370 | 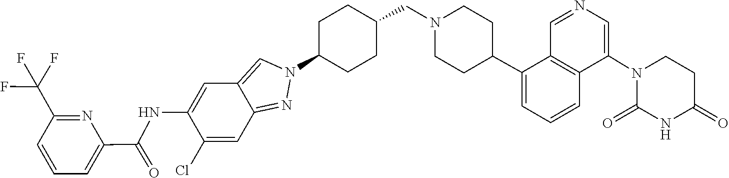 [SMILES: O=C1CCN(c2cncc3c(C4CCN(C[C@H]5CC[C@H](n6cc7cc(NC(=O)c8cccc(C(F)(F)F)n8)c(Cl)cc7n6)CC5)CC4)cccc23)C(=O)N1] |
| I-371 | 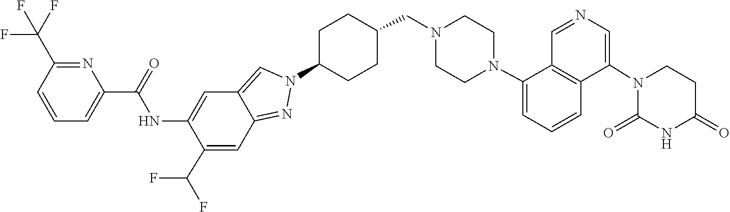 [SMILES: O=C1CCN(c2cncc3c(N4CCN(C[C@H]5CC[C@H](n6cc7cc(NC(=O)c8cccc(C(F)(F)F)n8)c(C(F)F)cc7n6)CC5)CC4)cccc23)C(=O)N1] |
| I-372 | 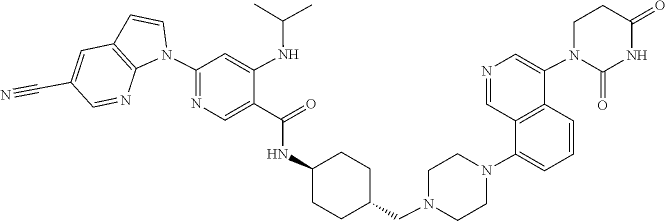 [SMILES: CC(C)Nc1cc(-n2ccc3cc(C#N)cnc32)ncc1C(=O)N[C@H]1CC[C@H](CN2CCN(c3cccc4c(N5CCC(=O)NC5=O)cncc34)CC2)CC1] |
| I-373 | 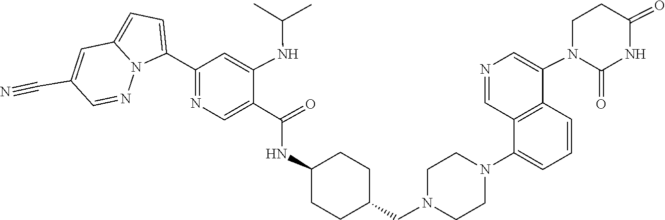 [SMILES: CC(C)Nc1cc(-c2ccc3cc(C#N)cnn23)ncc1C(=O)N[C@H]1CC[C@H](CN2CCN(c3cccc4c(N5CCC(=O)NC5=O)cncc34)CC2)CC1] |
| I-374 | 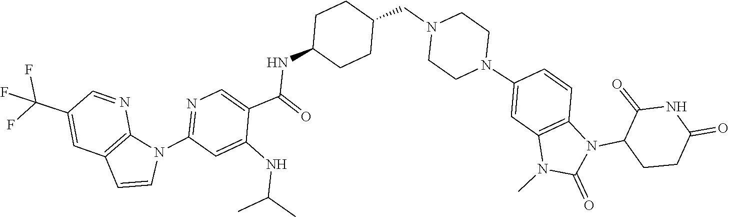 [SMILES: CC(C)Nc1cc(-n2ccc3cc(C(F)(F)F)cnc32)ncc1C(=O)N[C@H]1CC[C@H](CN2CCN(c3ccc4c(c3)n(C)c(=O)n4C3CCC(=O)NC3=O)CC2)CC1] |
| I-375 | 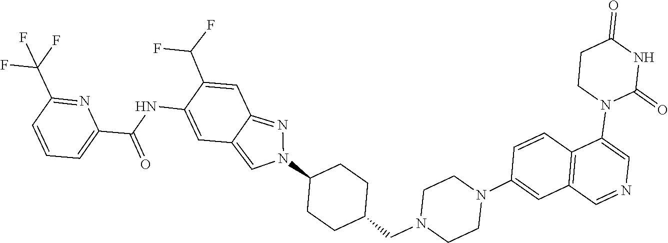 [SMILES: O=C1CCN(c2cncc3cc(N4CCN(C[C@H]5CC[C@H](n6cc7cc(NC(=O)c8cccc(C(F)(F)F)n8)c(C(F)F)cc7n6)CC5)CC4)ccc23)C(=O)N1] |
| I-376 | 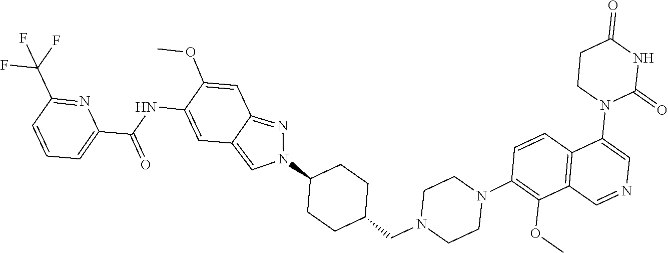 [SMILES: COc1cc2nn([C@H]3CC[C@H](CN4CCN(c5ccc6c(N7CCC(=O)NC7=O)cncc6c5OC)CC4)CC3)cc2cc1NC(=O)c1cccc(C(F)(F)F)n1] |
| I-377 | 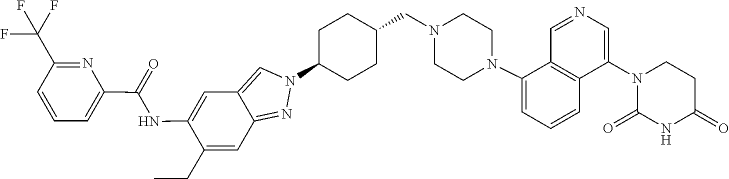 [SMILES: CCc1cc2nn([C@H]3CC[C@H](CN4CCN(c5cccc6c(N7CCC(=O)NC7=O)cncc56)CC4)CC3)cc2cc1NC(=O)c1cccc(C(F)(F)F)n1] |
| I-378 | 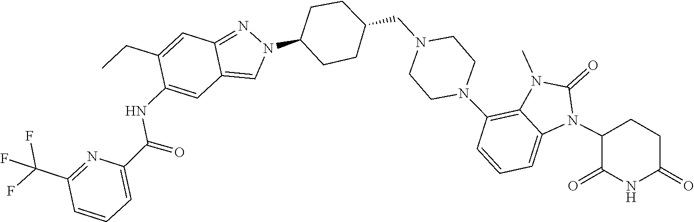 [SMILES: CCc1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-379 | 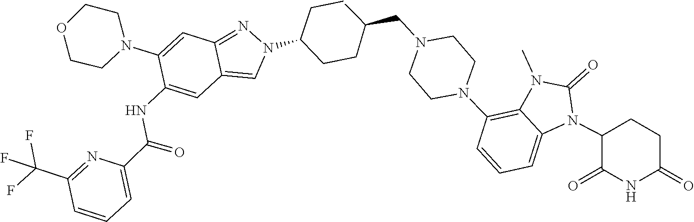 [SMILES: CC[C@H](CC[C@@H](C)CN1CCN(c2cccc3c2n(C)c(=O)n3C2CCC(=O)NC2=O)CC1)n1cc2cc(NC(=O)c3cccc(C(F)(F)F)n3)c(N3CCOCC3)cc2n1] |
| I-380 | 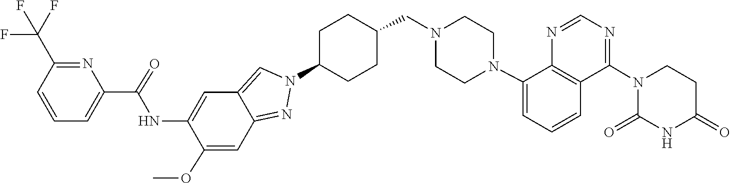 [SMILES: COc1cc2nn([C@H]3CC[C@H](CN4CCN(c5cccc6c(N7CCC(=O)NC7=O)ncnc56)CC4)CC3)cc2cc1NC(=O)c1cccc(C(F)(F)F)n1] |
| I-382 | 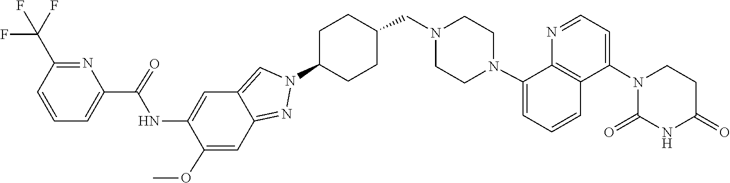 [SMILES: COc1cc2nn([C@H]3CC[C@H](CN4CCN(c5cccc6c(N7CCC(=O)NC7=O)ccnc56)CC4)CC3)cc2cc1NC(=O)c1cccc(C(F)(F)F)n1] |
| I-383 | 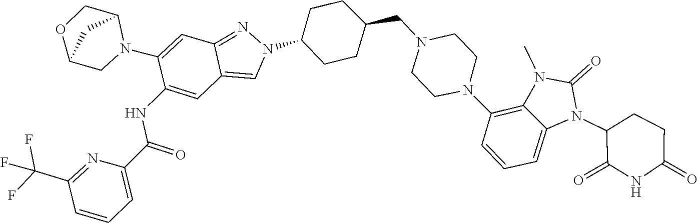 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(N7C[C@@H]8C[C@H]7CO8)cc6n5)CC4)CC3)c21] |
| I-384 | 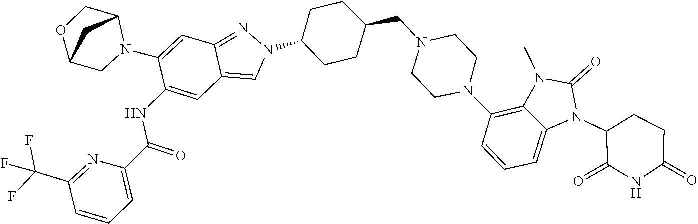 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(N7C[C@H]8C[C@@H]7CO8)cc6n5)CC4)CC3)c21] |
| I-385 | 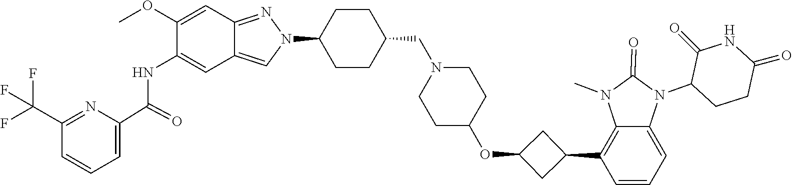 [SMILES: COc1cc2nn([C@H]3CC[C@H](CN4CCC(O[C@H]5C[C@@H](c6cccc7c6n(C)c(=O)n7C6CCC(=O)NC6=O)C5)CC4)CC3)cc2cc1NC(=O)c1cccc(C(F)(F)F)n1] |
| I-386 | 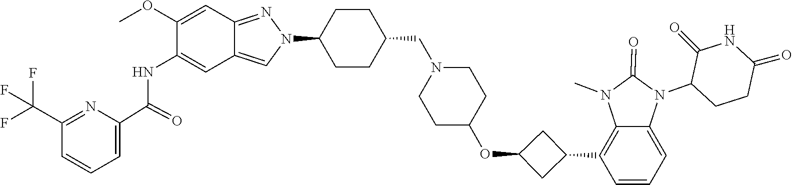 [SMILES: COc1cc2nn([C@H]3CC[C@H](CN4CCC(O[C@H]5C[C@H](c6cccc7c6n(C)c(=O)n7C6CCC(=O)NC6=O)C5)CC4)CC3)cc2cc1NC(=O)c1cccc(C(F)(F)F)n1] |
| I-387 | 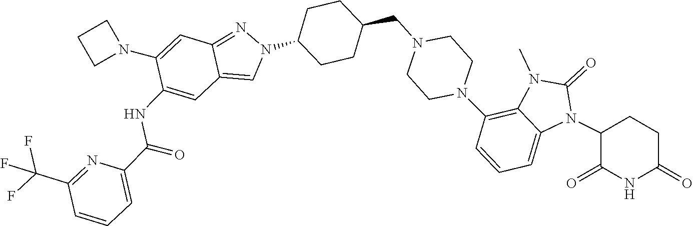 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(N7CCC7)cc6n5)CC4)CC3)c21] |
| I-388 | 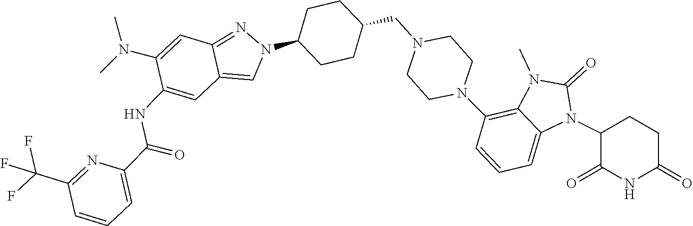 [SMILES: CN(C)c1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-389 | 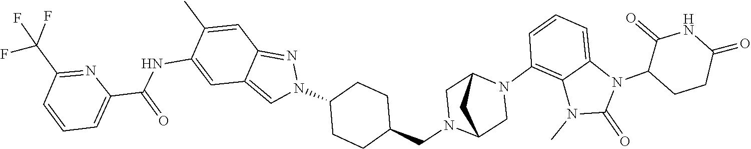 [SMILES: Cc1cc2nn([C@H]3CC[C@H](CN4C[C@H]5C[C@@H]4CN5c4cccc5c4n(C)c(=O)n5C4CCC(=O)NC4=O)CC3)cc2cc1NC(=O)c1cccc(C(F)(F)F)n1] |
| I-390 | 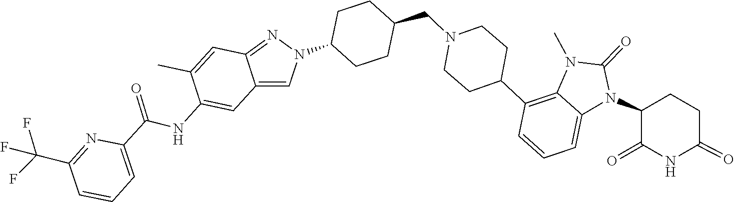 [SMILES: Cc1cc2nn([C@H]3CC[C@H](CN4CCC(c5cccc6c5n(C)c(=O)n6[C@H]5CCC(=O)NC5=O)CC4)CC3)cc2cc1NC(=O)c1cccc(C(F)(F)F)n1] |
| I-391 | 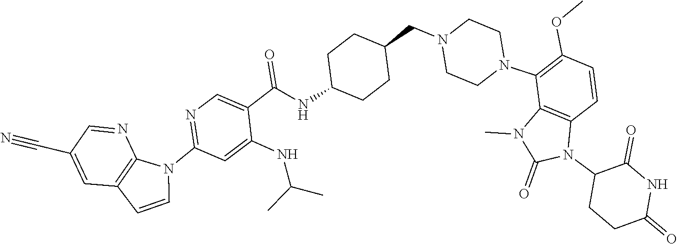 [SMILES: COc1ccc2c(c1N1CCN(C[C@H]3CC[C@H](NC(=O)c4cnc(-n5ccc6cc(C#N)cnc65)cc4NC(C)C)CC3)CC1)n(C)c(=O)n2C1CCC(=O)NC1=O] |
| I-392 | 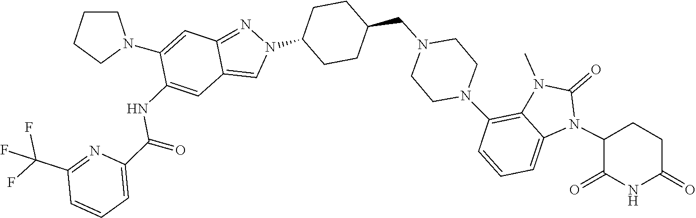 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(N7CCCC7)cc6n5)CC4)CC3)c21] |
| I-393 | 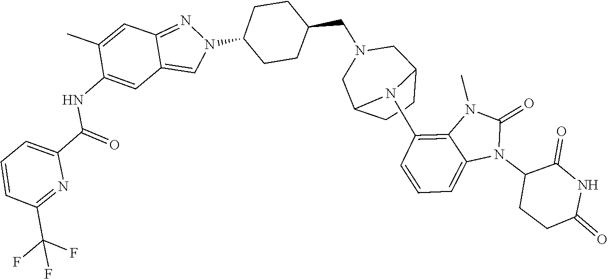 [SMILES: CCC1CN(C[C@H]2CC[C@H](n3cc4cc(NC(=O)c5cccc(C(F)(F)F)n5)c(C)cc4n3)CC2)CC(CC)N1c1cccc2c1n(C)c(=O)n2C1CCC(=O)NC1=O] |
| I-394 | 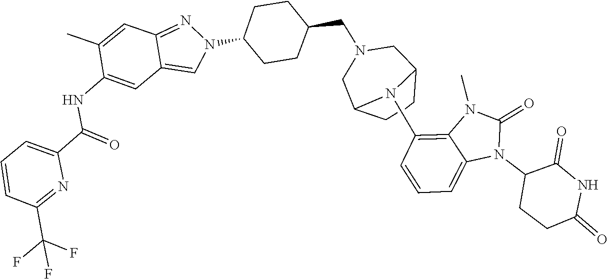 [SMILES: CCC1CN(C[C@H]2CC[C@H](n3cc4cc(NC(=O)c5cccc(C(F)(F)F)n5)c(C)cc4n3)CC2)CC(CC)N1c1cccc2c1n(C)c(=O)n2C1CCC(=O)NC1=O] |
| I-395 | 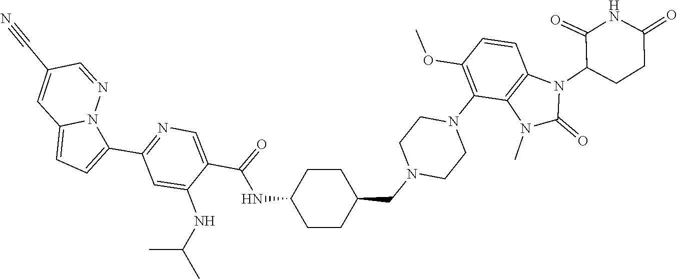 [SMILES: COc1ccc2c(c1N1CCN(C[C@H]3CC[C@H](NC(=O)c4cnc(-c5ccc6cc(C#N)cnn56)cc4NC(C)C)CC3)CC1)n(C)c(=O)n2C1CCC(=O)NC1=O] |
| I-396 | 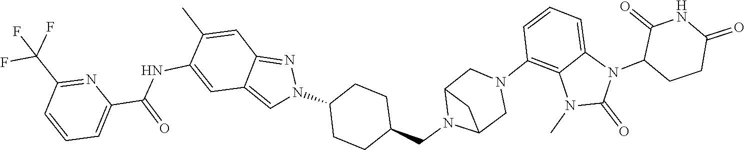 [SMILES: Cc1cc2nn([C@H]3CC[C@H](CN4C5CC4CN(c4cccc6c4n(C)c(=O)n6C4CCC(=O)NC4=O)C5)CC3)cc2cc1NC(=O)c1cccc(C(F)(F)F)n1] |
| I-397 | 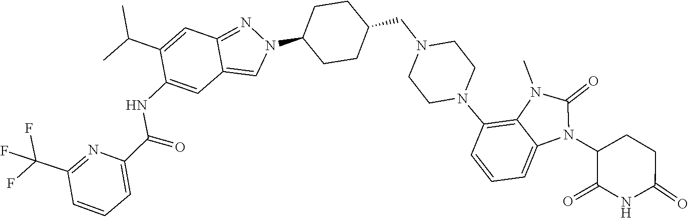 [SMILES: CC(C)c1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-398 | 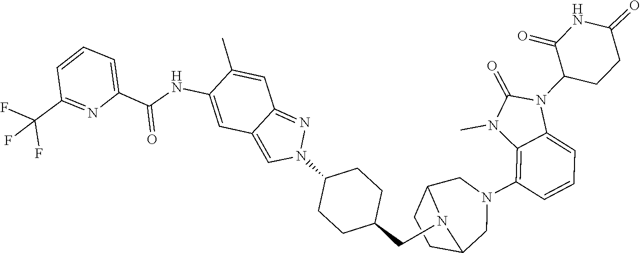 [SMILES: Cc1cc2nn([C@H]3CC[C@H](CN4C5CCC4CN(c4cccc6c4n(C)c(=O)n6C4CCC(=O)NC4=O)C5)CC3)cc2cc1NC(=O)c1cccc(C(F)(F)F)n1] |
| I-399 | 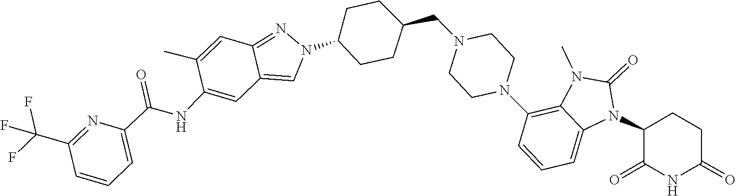 [SMILES: Cc1cc2nn([C@H]3CC[C@H](CN4CCN(c5cccc6c5n(C)c(=O)n6[C@H]5CCC(=O)NC5=O)CC4)CC3)cc2cc1NC(=O)c1cccc(C(F)(F)F)n1] |
| I-400 | 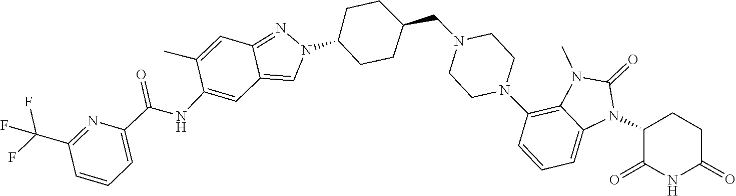 [SMILES: Cc1cc2nn([C@H]3CC[C@H](CN4CCN(c5cccc6c5n(C)c(=O)n6[C@@H]5CCC(=O)NC5=O)CC4)CC3)cc2cc1NC(=O)c1cccc(C(F)(F)F)n1] |
| I-401 | 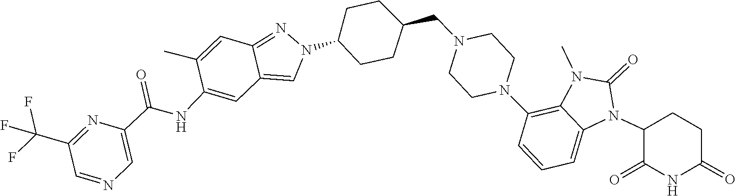 [SMILES: Cc1cc2nn([C@H]3CC[C@H](CN4CCN(c5cccc6c5n(C)c(=O)n6C5CCC(=O)NC5=O)CC4)CC3)cc2cc1NC(=O)c1cncc(C(F)(F)F)n1] |
| I-402 | 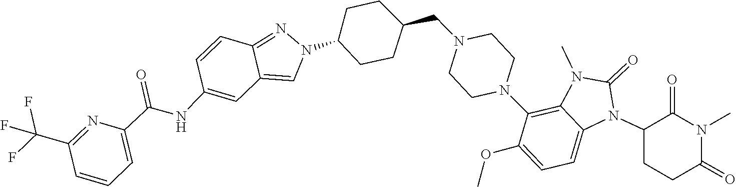 [SMILES: COc1ccc2c(c1N1CCN(C[C@H]3CC[C@H](n4cc5cc(NC(=O)c6cccc(C(F)(F)F)n6)ccc5n4)CC3)CC1)n(C)c(=O)n2C1CCC(=O)N(C)C1=O] |
| I-403 | 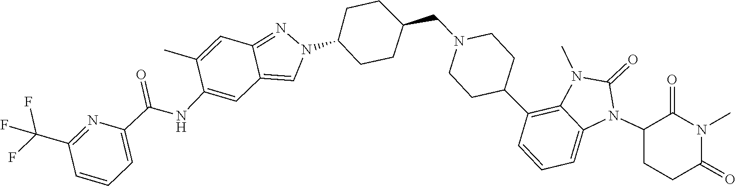 [SMILES: Cc1cc2nn([C@H]3CC[C@H](CN4CCC(c5cccc6c5n(C)c(=O)n6C5CCC(=O)N(C)C5=O)CC4)CC3)cc2cc1NC(=O)c1cccc(C(F)(F)F)n1] |
| I-404 | 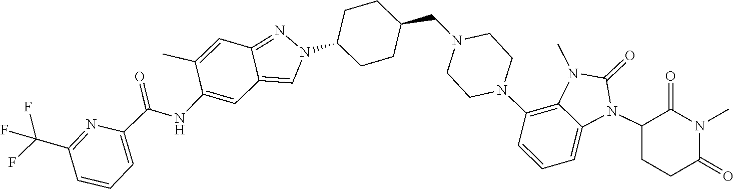 [SMILES: Cc1cc2nn([C@H]3CC[C@H](CN4CCN(c5cccc6c5n(C)c(=O)n6C5CCC(=O)N(C)C5=O)CC4)CC3)cc2cc1NC(=O)c1cccc(C(F)(F)F)n1] |
| I-405 | 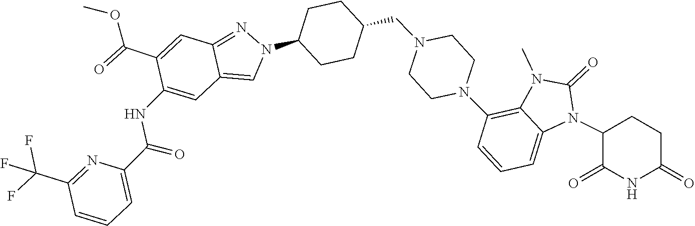 [SMILES: COC(=O)c1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-406 | 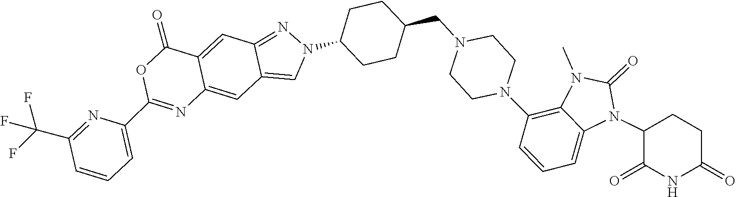 [SMILES: Cn1c(=O)n(C2CCC(=O)NC2=O)c2cccc(N3CCN(C[C@H]4CC[C@H](n5cc6cc7nc(-c8cccc(C(F)(F)F)n8)oc(=O)c7cc6n5)CC4)CC3)c21] |
| I-407 | 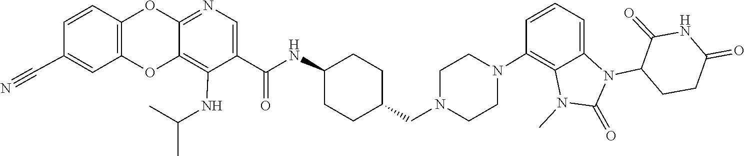 [SMILES: CC(C)Nc1c(C(=O)N[C@H]2CC[C@H](CN3CCN(c4cccc5c4n(C)c(=O)n5C4CCC(=O)NC4=O)CC3)CC2)cnc2c1Oc1cc(C#N)ccc1O2] |
| I-408 | 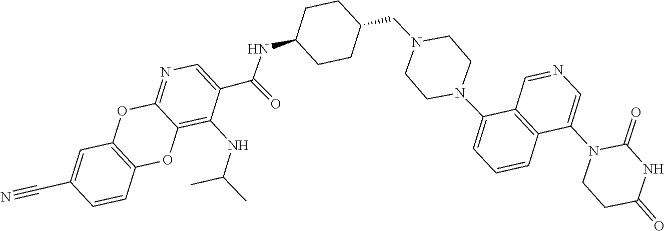 [SMILES: CC(C)Nc1c(C(=O)N[C@H]2CC[C@H](CN3CCN(c4cccc5c(N6CCC(=O)NC6=O)cncc45)CC3)CC2)cnc2c1Oc1ccc(C#N)cc1O2] |
| I-409 | 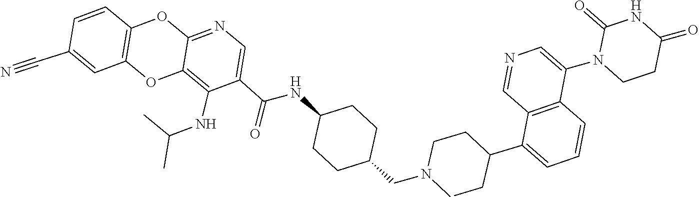 [SMILES: CCC(CCN(C)C[C@H]1CC[C@H](NC(=O)c2cnc3c(c2NC(C)C)Oc2cc(C#N)ccc2O3)CC1)c1cccc2c(N3CCC(=O)NC3=O)cncc12] |
| I-410 | 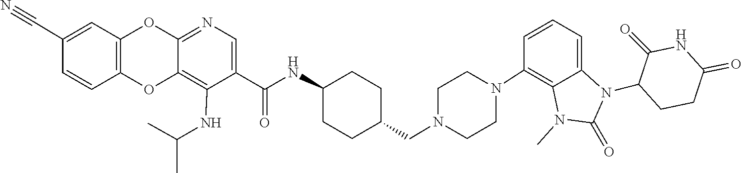 [SMILES: CC(C)Nc1c(C(=O)N[C@H]2CC[C@H](CN3CCN(c4cccc5c4n(C)c(=O)n5C4CCC(=O)NC4=O)CC3)CC2)cnc2c1Oc1ccc(C#N)cc1O2] |
| I-411 | 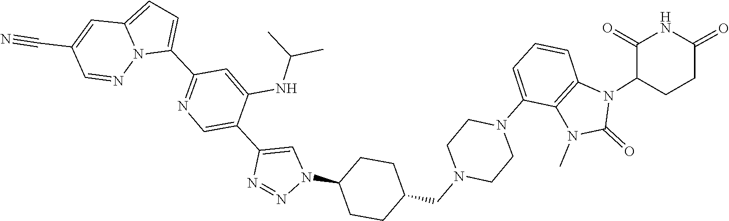 [SMILES: CC(C)Nc1cc(-c2ccc3cc(C#N)cnn23)ncc1-c1cn([C@H]2CC[C@H](CN3CCN(c4cccc5c4n(C)c(=O)n5C4CCC(=O)NC4=O)CC3)CC2)nn1] |
| I-412 | 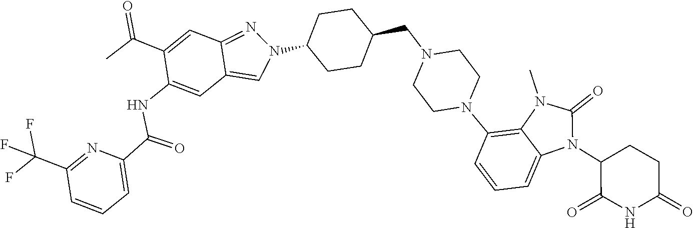 [SMILES: CC(=O)c1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1] |
| I-413 | 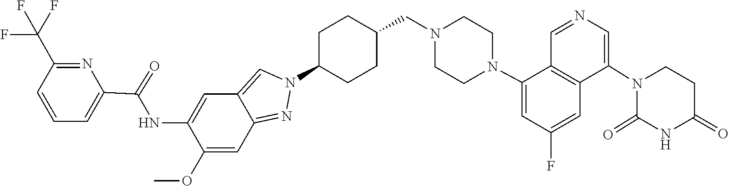 [SMILES: COc1cc2nn([C@H]3CC[C@H](CN4CCN(c5cc(F)cc6c(N7CCC(=O)NC7=O)cncc56)CC4)CC3)cc2cc1NC(=O)c1cccc(C(F)(F)F)n1] |
| I-414 | 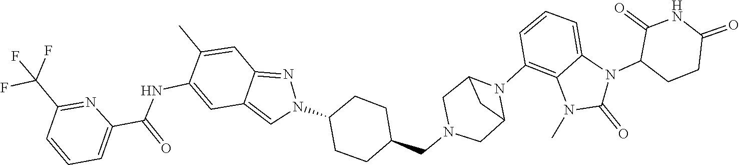 [SMILES: Cc1cc2nn([C@H]3CC[C@H](CN4CC5CC(C4)N5c4cccc5c4n(C)c(=O)n5C4CCC(=O)NC4=O)CC3)cc2cc1NC(=O)c1cccc(C(F)(F)F)n1] |
| I-415 | 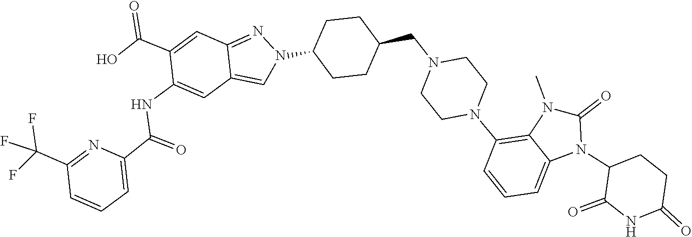 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(C(=O)O)cc6n5)CC4)CC3)c21] |
| I-416 | 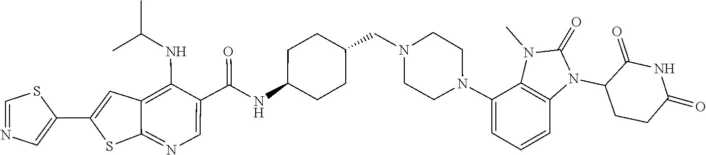 [SMILES: CC(C)Nc1c(C(=O)N[C@H]2CC[C@H](CN3CCN(c4cccc5c4n(C)c(=O)n5C4CCC(=O)NC4=O)CC3)CC2)cnc2sc(-c3cncs3)cc12] |
| I-417 | 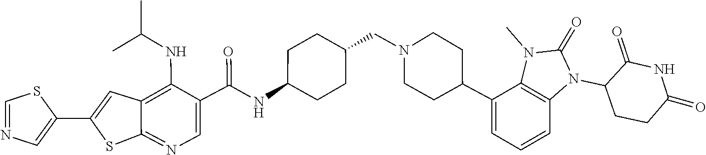 [SMILES: CC(C)Nc1c(C(=O)N[C@H]2CC[C@H](CN3CCC(c4cccc5c4n(C)c(=O)n5C4CCC(=O)NC4=O)CC3)CC2)cnc2sc(-c3cncs3)cc12] |
| I-418 | 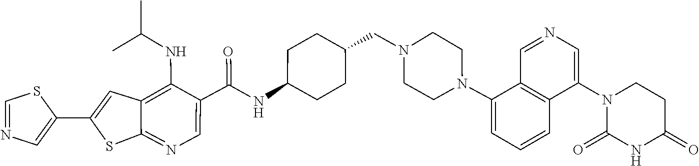 [SMILES: CC(C)Nc1c(C(=O)N[C@H]2CC[C@H](CN3CCN(c4cccc5c(N6CCC(=O)NC6=O)cncc45)CC3)CC2)cnc2sc(-c3cncs3)cc12] |
| I-419 | 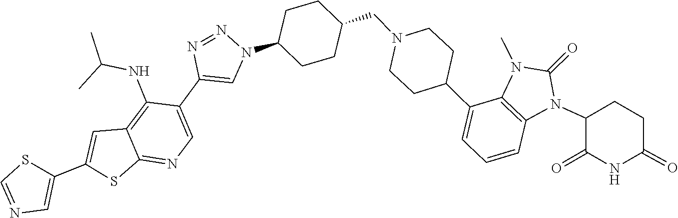 [SMILES: CC(C)Nc1c(-c2cn([C@H]3CC[C@H](CN4CCC(c5cccc6c5n(C)c(=O)n6C5CCC(=O)NC5=O)CC4)CC3)nn2)cnc2sc(-c3cncs3)cc12] |
| I-420 | 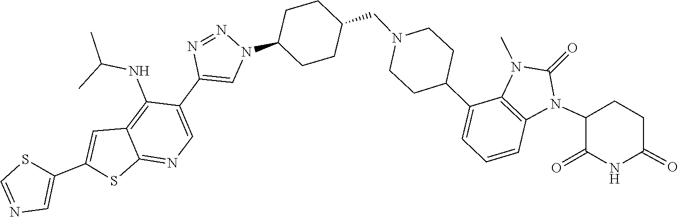 [SMILES: CC(C)Nc1c(-c2cn([C@H]3CC[C@H](CN4CCC(c5cccc6c5n(C)c(=O)n6C5CCC(=O)NC5=O)CC4)CC3)nn2)cnc2sc(-c3cncs3)cc12] |
| I-421 | 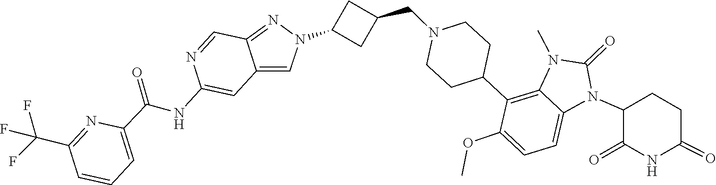 [SMILES: COc1ccc2c(c1C1CCN(C[C@H]3C[C@H](n4cc5cc(NC(=O)c6cccc(C(F)(F)F)n6)ncc5n4)C3)CC1)n(C)c(=O)n2C1CCC(=O)NC1=O] |
| I-422 | 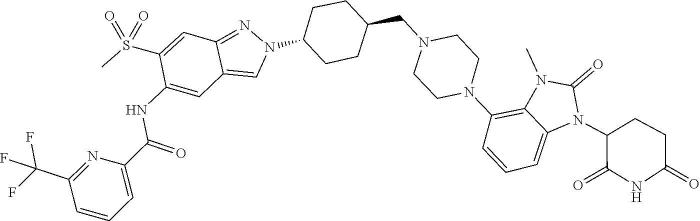 [SMILES: Cn1c(=O)n(C2CCC(=O)NC2=O)c2cccc(N3CCN(C[C@H]4CC[C@H](n5cc6cc(NC(=O)c7cccc(C(F)(F)F)n7)c(S(C)(=O)=O)cc6n5)CC4)CC3)c21] |
| I-423 | 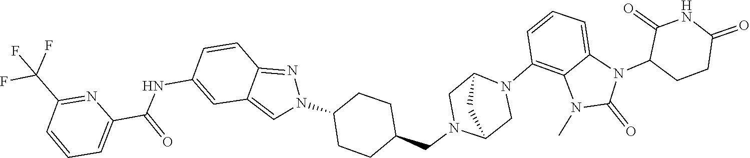 [SMILES: Cn1c(=O)n(C2CCC(=O)NC2=O)c2cccc(N3C[C@@H]4C[C@H]3CN4C[C@H]3CC[C@H](n4cc5cc(NC(=O)c6cccc(C(F)(F)F)n6)ccc5n4)CC3)c21] |
| I-424 | 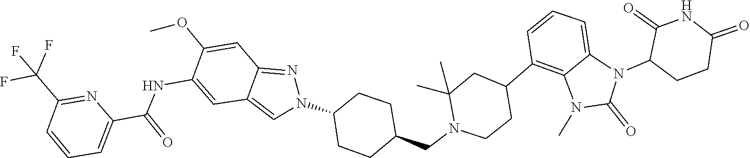 [SMILES: COc1cc2nn([C@H]3CC[C@H](CN4CCC(c5cccc6c5n(C)c(=O)n6C5CCC(=O)NC5=O)CC4(C)C)CC3)cc2cc1NC(=O)c1cccc(C(F)(F)F)n1] |
| I-425 | 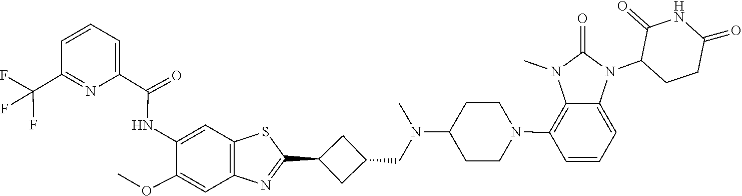 [SMILES: COc1cc2nc([C@H]3C[C@H](CN(C)C4CCN(c5cccc6c5n(C)c(=O)n6C5CCC(=O)NC5=O)CC4)C3)sc2cc1NC(=O)c1cccc(C(F)(F)F)n1] |
| I-426 | 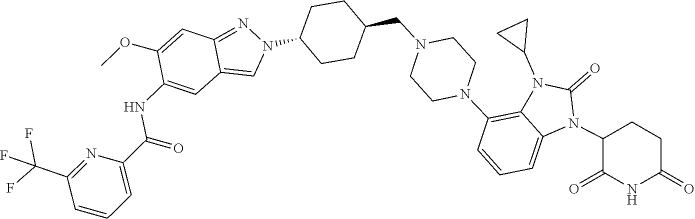 [SMILES: COc1cc2nn([C@H]3CC[C@H](CN4CCN(c5cccc6c5n(C5CC5)c(=O)n6C5CCC(=O)NC5=O)CC4)CC3)cc2cc1NC(=O)c1cccc(C(F)(F)F)n1] |
| I-427 | 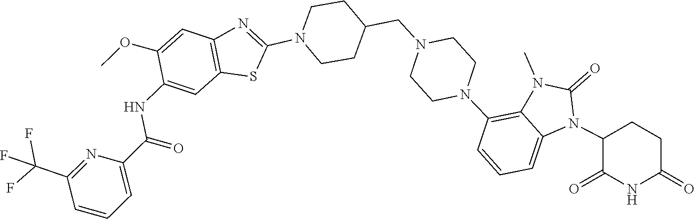 [SMILES: COc1cc2nc(N3CCC(CN4CCN(c5cccc6c5n(C)c(=O)n6C5CCC(=O)NC5=O)CC4)CC3)sc2cc1NC(=O)c1cccc(C(F)(F)F)n1] |
| I-428 | 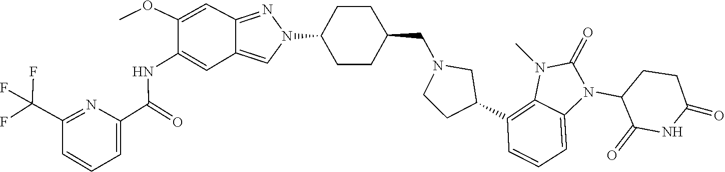 [SMILES: COc1cc2nn([C@H]3CC[C@H](CN4CC[C@@H](c5cccc6c5n(C)c(=O)n6C5CCC(=O)NC5=O)C4)CC3)cc2cc1NC(=O)c1cccc(C(F)(F)F)n1] |
| I-429 | 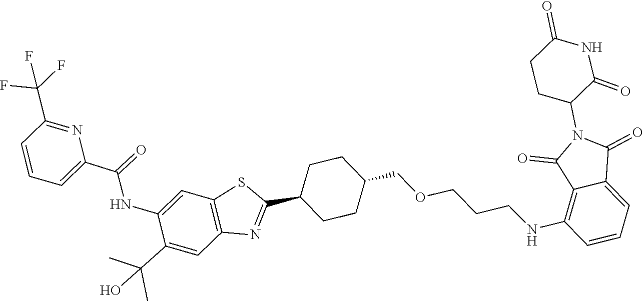 [SMILES: CC(C)(O)c1cc2nc([C@H]3CC[C@H](COCCCNc4cccc5c4C(=O)N(C4CCC(=O)NC4=O)C5=O)CC3)sc2cc1NC(=O)c1cccc(C(F)(F)F)n1] |
| I-430 | 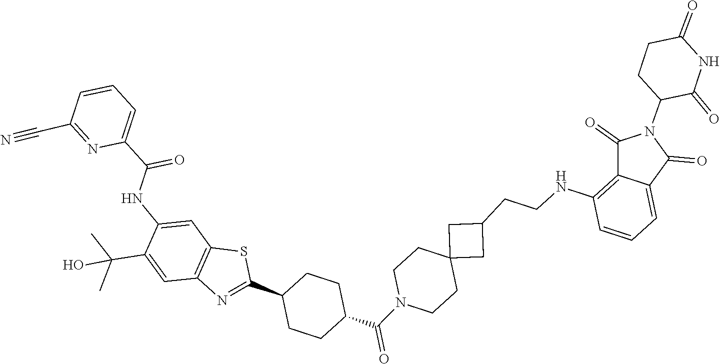 [SMILES: CC(C)(O)c1cc2nc([C@H]3CC[C@H](C(=O)N4CCC5(CC4)CC(CCNc4cccc6c4C(=O)N(C4CCC(=O)NC4=O)C6=O)C5)CC3)sc2cc1NC(=O)c1cccc(C#N)n1] |
| I-431 | 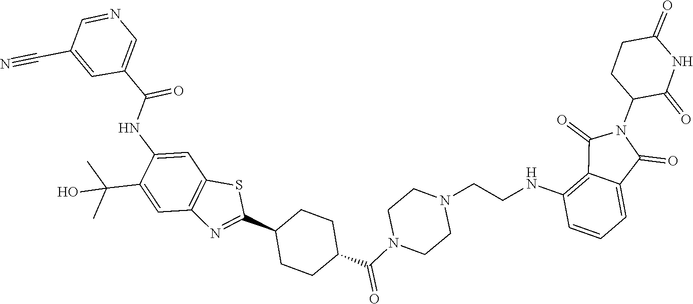 [SMILES: CC(C)(O)c1cc2nc([C@H]3CC[C@H](C(=O)N4CCN(CCNc5cccc6c5C(=O)N(C5CCC(=O)NC5=O)C6=O)CC4)CC3)sc2cc1NC(=O)c1cncc(C#N)c1] |
| I-432 | 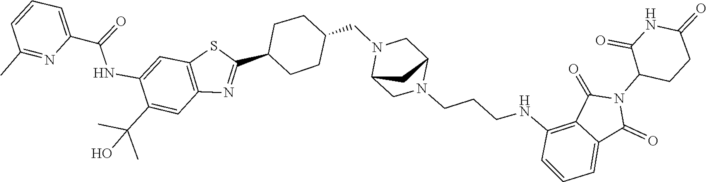 [SMILES: Cc1cccc(C(=O)Nc2cc3sc([C@H]4CC[C@H](CN5C[C@H]6C[C@@H]5CN6CCCNc5cccc6c5C(=O)N(C5CCC(=O)NC5=O)C6=O)CC4)nc3cc2C(C)(C)O)n1] |
| I-433 | 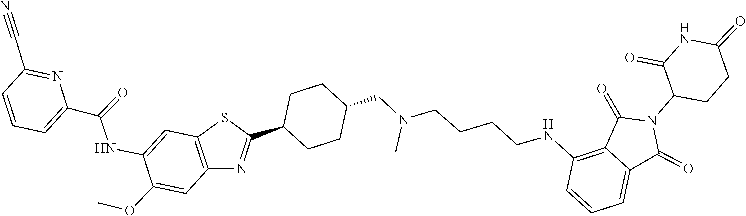 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cccc(C#N)n1] |
| I-434 | 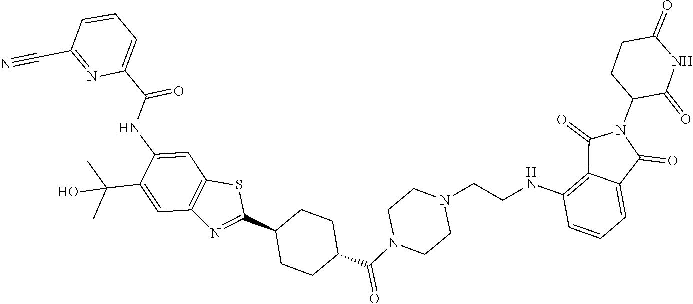 [SMILES: CC(C)(O)c1cc2nc([C@H]3CC[C@H](C(=O)N4CCN(CCNc5cccc6c5C(=O)N(C5CCC(=O)NC5=O)C6=O)CC4)CC3)sc2cc1NC(=O)c1cccc(C#N)n1] |
| I-435 | 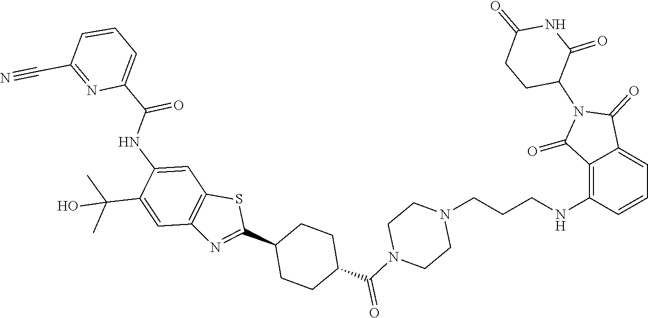 [SMILES: CC(C)(O)c1cc2nc([C@H]3CC[C@H](C(=O)N4CCN(CCCNc5cccc6c5C(=O)N(C5CCC(=O)NC5=O)C6=O)CC4)CC3)sc2cc1NC(=O)c1cccc(C#N)n1] |
| I-436 | 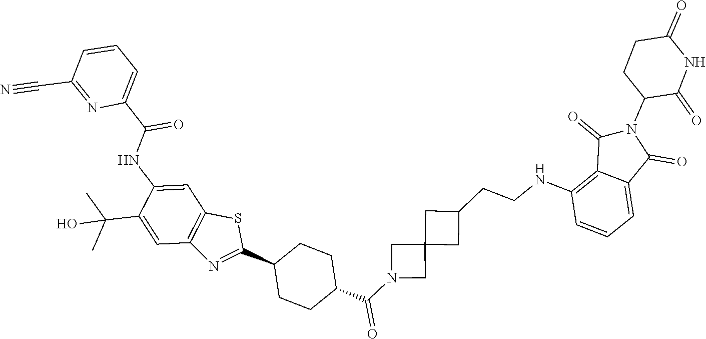 [SMILES: CC(C)(O)c1cc2nc([C@H]3CC[C@H](C(=O)N4CC5(CC(CCNc6cccc7c6C(=O)N(C6CCC(=O)NC6=O)C7=O)C5)C4)CC3)sc2cc1NC(=O)c1cccc(C#N)n1] |
| I-437 | 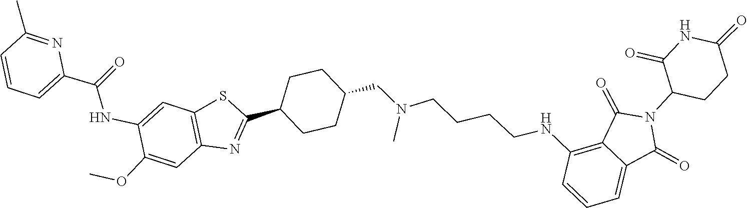 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cccc(C)n1] |
| I-438 | 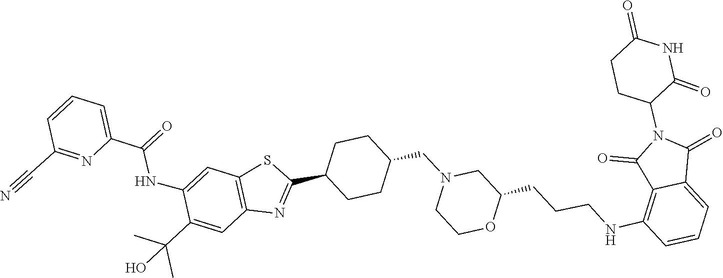 [SMILES: CC(C)(O)c1cc2nc([C@H]3CC[C@H](CN4CCO[C@@H](CCCNc5cccc6c5C(=O)N(C5CCC(=O)NC5=O)C6=O)C4)CC3)sc2cc1NC(=O)c1cccc(C#N)n1] |
| I-439 | 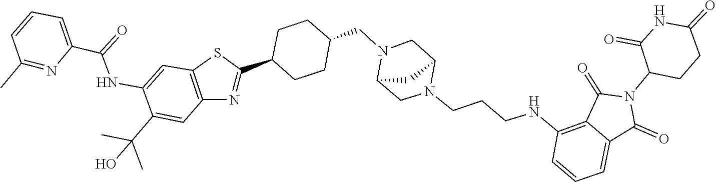 [SMILES: Cc1cccc(C(=O)Nc2cc3sc([C@H]4CC[C@H](CN5C[C@@H]6C[C@H]5CN6CCCNc5cccc6c5C(=O)N(C5CCC(=O)NC5=O)C6=O)CC4)nc3cc2C(C)(C)O)n1] |
| I-440 | 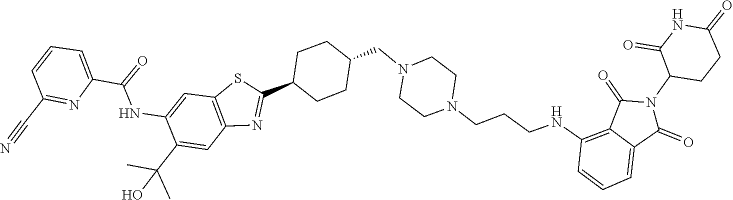 [SMILES: CC(C)(O)c1cc2nc([C@H]3CC[C@H](CN4CCN(CCCNc5cccc6c5C(=O)N(C5CCC(=O)NC5=O)C6=O)CC4)CC3)sc2cc1NC(=O)c1cccc(C#N)n1] |
| I-441 | 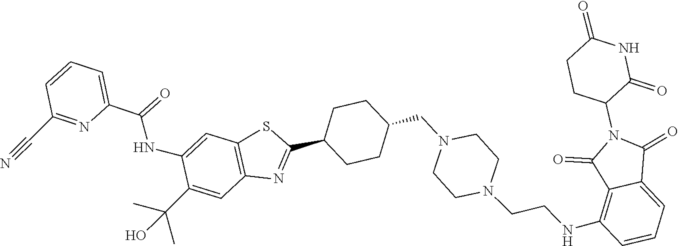 [SMILES: CC(C)(O)c1cc2nc([C@H]3CC[C@H](CN4CCN(CCNc5cccc6c5C(=O)N(C5CCC(=O)NC5=O)C6=O)CC4)CC3)sc2cc1NC(=O)c1cccc(C#N)n1] |
| I-442 | 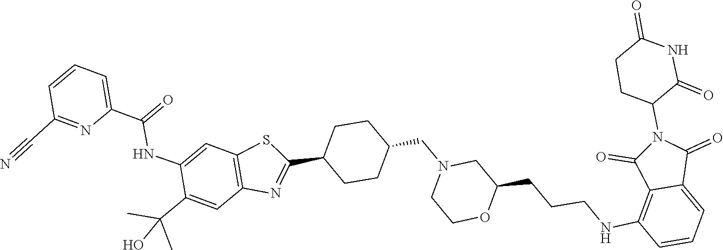 [SMILES: CC(C)(O)c1cc2nc([C@H]3CC[C@H](CN4CCO[C@H](CCCNc5cccc6c5C(=O)N(C5CCC(=O)NC5=O)C6=O)C4)CC3)sc2cc1NC(=O)c1cccc(C#N)n1] |
| I-443 | 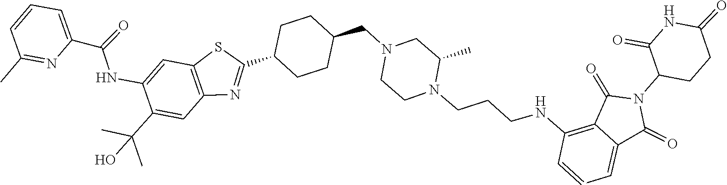 [SMILES: C=C/C(NCCCN1CCN(C[C@H]2CC[C@H](c3nc4cc(C(C)(C)O)c(NC(=O)c5cccc(C)n5)cc4s3)CC2)C[C@@H]1C)=C1/C(=O)N(C2CCC(=O)NC2=O)C(=O)/C1=C/C] |
| I-444 | 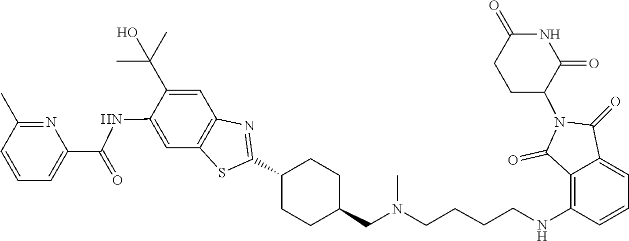 [SMILES: Cc1cccc(C(=O)Nc2cc3sc([C@H]4CC[C@H](CN(C)CCCCNc5cccc6c5C(=O)N(C5CCC(=O)NC5=O)C6=O)CC4)nc3cc2C(C)(C)O)n1] |
| I-445 | 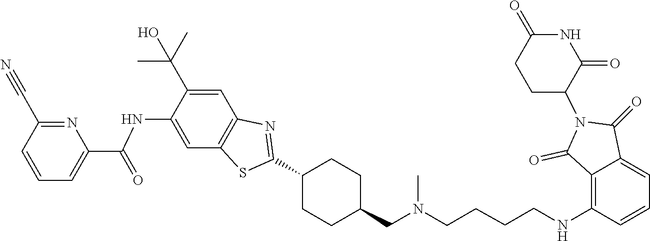 [SMILES: CN(CCCCNc1cccc2c1C(=O)N(C1CCC(=O)NC1=O)C2=O)C[C@H]1CC[C@H](c2nc3cc(C(C)(C)O)c(NC(=O)c4cccc(C#N)n4)cc3s2)CC1] |
| I-446 | 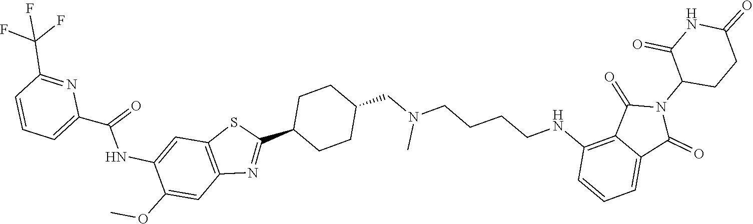 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cccc(C(F)(F)F)n1] |
| I-447 | 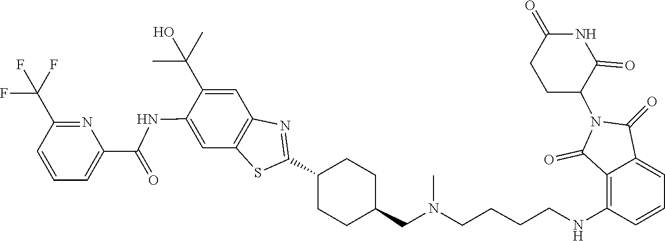 [SMILES: CN(CCCCNc1cccc2c1C(=O)N(C1CCC(=O)NC1=O)C2=O)C[C@H]1CC[C@H](c2nc3cc(C(C)(C)O)c(NC(=O)c4cccc(C(F)(F)F)n4)cc3s2)CC1] |
| I-448 | 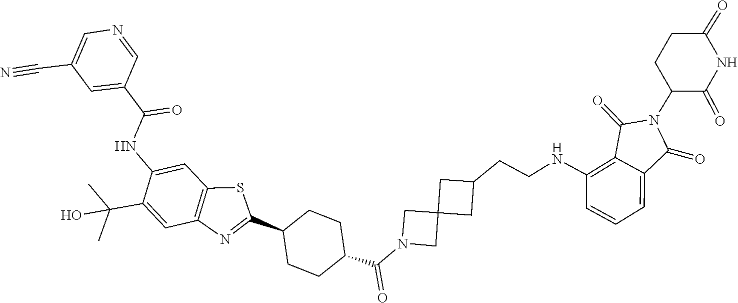 [SMILES: CC(C)(O)c1cc2nc([C@H]3CC[C@H](C(=O)N4CC5(CC(CCNc6cccc7c6C(=O)N(C6CCC(=O)NC6=O)C7=O)C5)C4)CC3)sc2cc1NC(=O)c1cncc(C#N)c1] |
| I-449 | 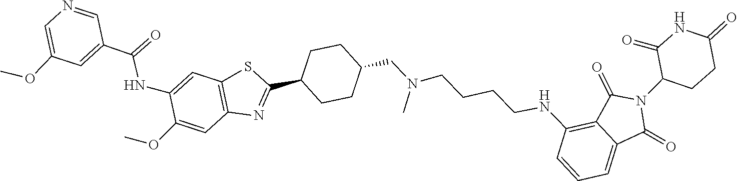 [SMILES: COc1cncc(C(=O)Nc2cc3sc([C@H]4CC[C@H](CN(C)CCCCNc5cccc6c5C(=O)N(C5CCC(=O)NC5=O)C6=O)CC4)nc3cc2OC)c1] |
| I-450 | 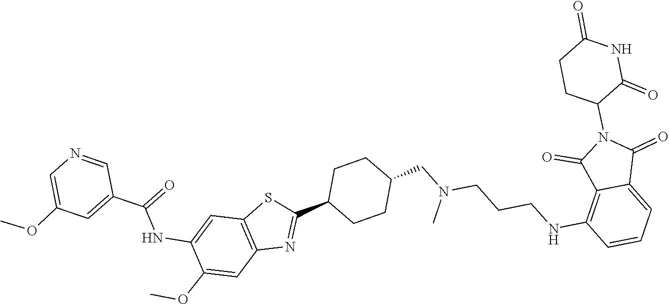 [SMILES: COc1cncc(C(=O)Nc2cc3sc([C@H]4CC[C@H](CN(C)CCCNc5cccc6c5C(=O)N(C5CCC(=O)NC5=O)C6=O)CC4)nc3cc2OC)c1] |
| I-451 | 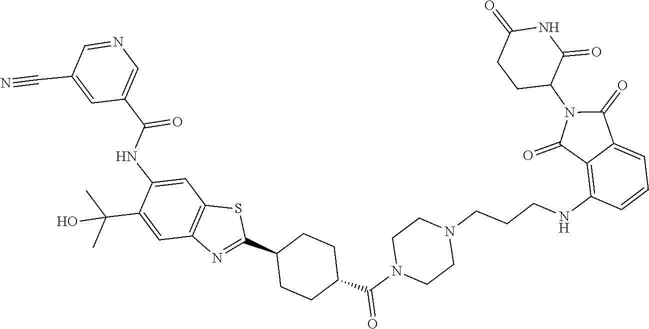 [SMILES: CC(C)(O)c1cc2nc([C@H]3CC[C@H](C(=O)N4CCN(CCCNc5cccc6c5C(=O)N(C5CCC(=O)NC5=O)C6=O)CC4)CC3)sc2cc1NC(=O)c1cncc(C#N)c1] |
| I-452 | 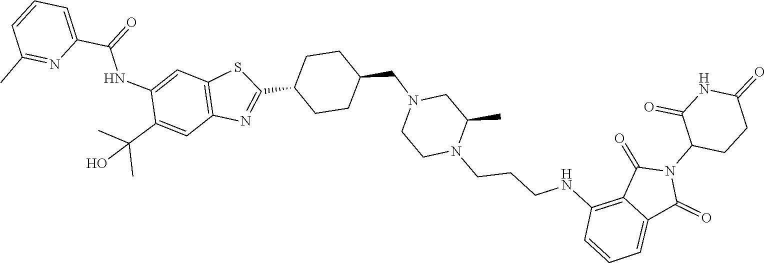 [SMILES: Cc1cccc(C(=O)Nc2cc3sc([C@H]4CC[C@H](CN5CCN(CCCNc6cccc7c6C(=O)N(C6CCC(=O)NC6=O)C7=O)[C@H](C)C5)CC4)nc3cc2C(C)(C)O)n1] |
| I-453 | 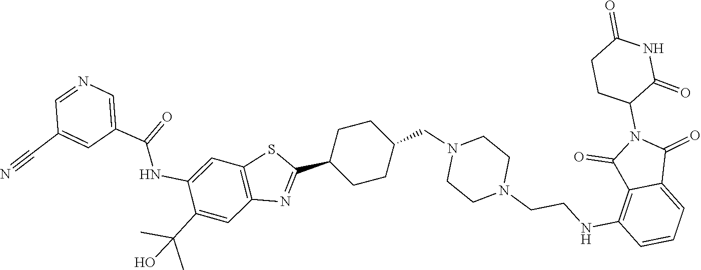 [SMILES: CC(C)(O)c1cc2nc([C@H]3CC[C@H](CN4CCN(CCNc5cccc6c5C(=O)N(C5CCC(=O)NC5=O)C6=O)CC4)CC3)sc2cc1NC(=O)c1cncc(C#N)c1] |
| I-454 | 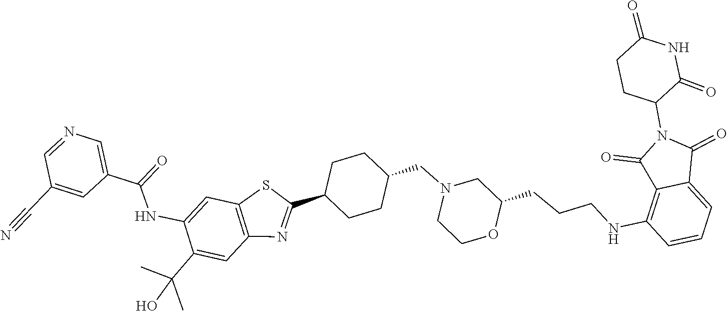 [SMILES: CC(C)(O)c1cc2nc([C@H]3CC[C@H](CN4CCO[C@@H](CCCNc5cccc6c5C(=O)N(C5CCC(=O)NC5=O)C6=O)C4)CC3)sc2cc1NC(=O)c1cncc(C#N)c1] |
| I-455 | 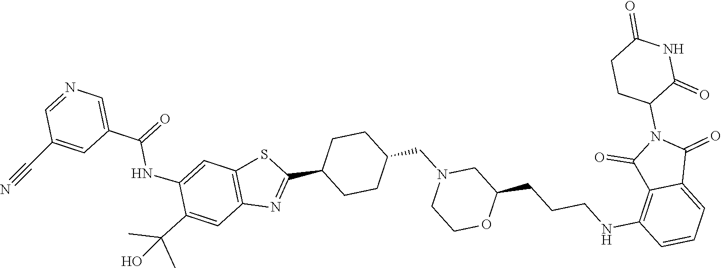 [SMILES: CC(C)(O)c1cc2nc([C@H]3CC[C@H](CN4CCO[C@H](CCCNc5cccc6c5C(=O)N(C5CCC(=O)NC5=O)C6=O)C4)CC3)sc2cc1NC(=O)c1cncc(C#N)c1] |
| I-456 | 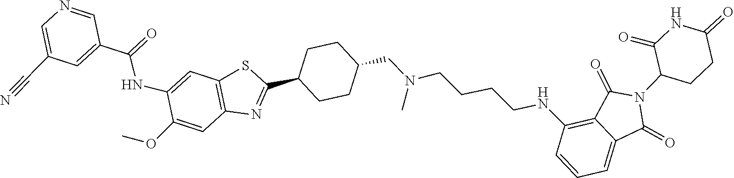 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cncc(C#N)c1] |
| I-457 | 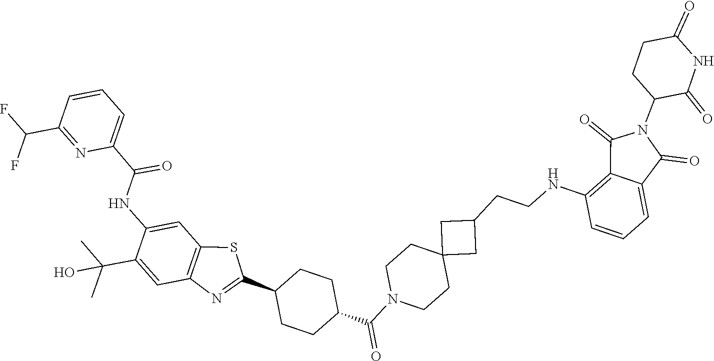 [SMILES: CC(C)(O)c1cc2nc([C@H]3CC[C@H](C(=O)N4CCC5(CC4)CC(CCNc4cccc6c4C(=O)N(C4CCC(=O)NC4=O)C6=O)C5)CC3)sc2cc1NC(=O)c1cccc(C(F)F)n1] |
| I-458 | 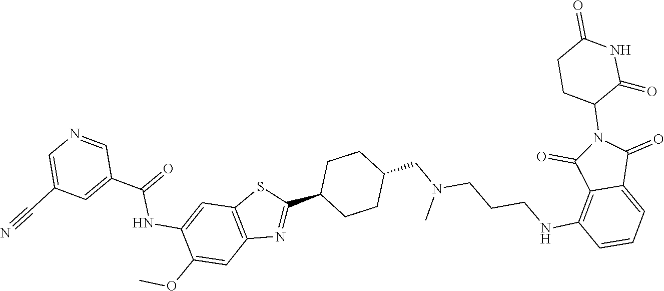 [SMILES: COc1cc2nc([C@H]3CC[C@H](CN(C)CCCNc4cccc5c4C(=O)N(C4CCC(=O)NC4=O)C5=O)CC3)sc2cc1NC(=O)c1cncc(C#N)c1] |
| I-459 | 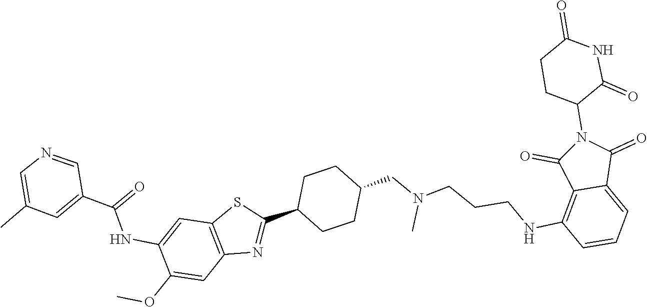 [SMILES: COc1cc2nc([C@H]3CC[C@H](CN(C)CCCNc4cccc5c4C(=O)N(C4CCC(=O)NC4=O)C5=O)CC3)sc2cc1NC(=O)c1cncc(C)c1] |
| I-460 | 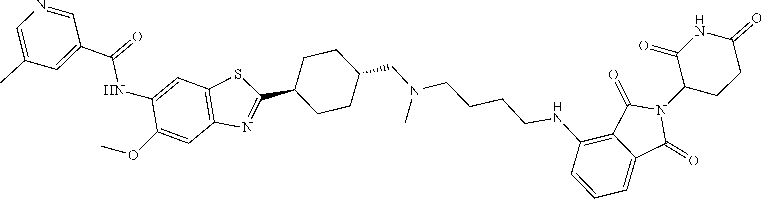 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cncc(C)c1] |
| I-461 | 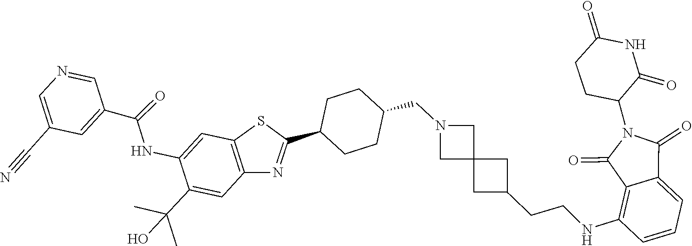 [SMILES: CC(C)(O)c1cc2nc([C@H]3CC[C@H](CN4CC5(CC(CCNc6cccc7c6C(=O)N(C6CCC(=O)NC6=O)C7=O)C5)C4)CC3)sc2cc1NC(=O)c1cncc(C#N)c1] |
| I-462 | 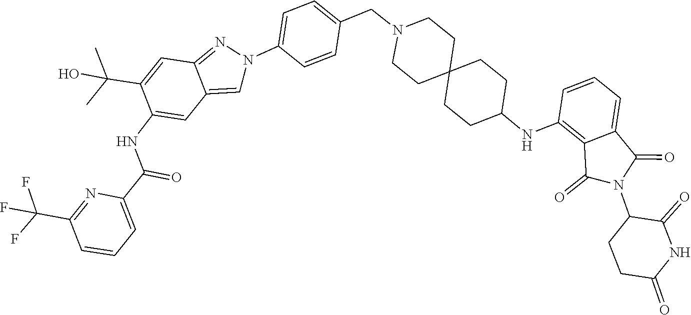 [SMILES: CC(C)(O)c1cc2nn(-c3ccc(CN4CCC5(CCC(Nc6cccc7c6C(=O)N(C6CCC(=O)NC6=O)C7=O)CC5)CC4)cc3)cc2cc1NC(=O)c1cccc(C(F)(F)F)n1] |
| I-463 | 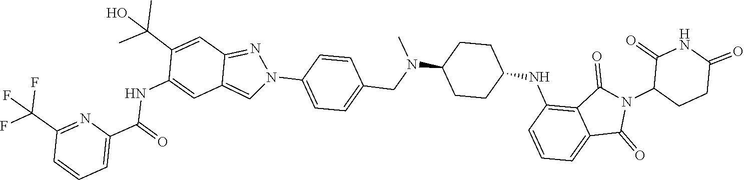 |
| I-464 | 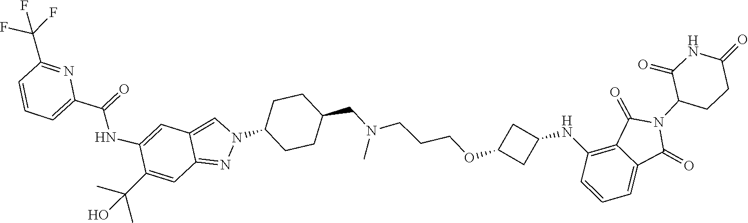 [SMILES: CN(CCCO[C@H]1C[C@@H](Nc2cccc3c2C(=O)N(C2CCC(=O)NC2=O)C3=O)C1)C[C@H]1CC[C@H](n2cc3cc(NC(=O)c4cccc(C(F)(F)F)n4)c(C(C)(C)O)cc3n2)CC1] |
| I-465 | 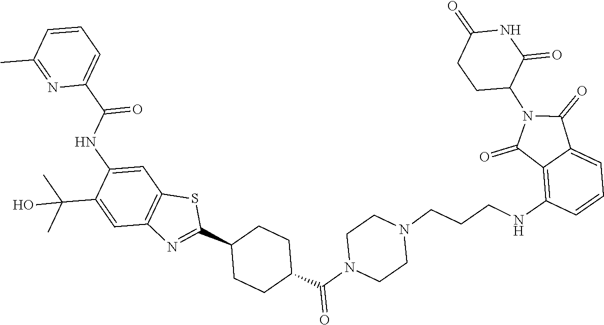 [SMILES: Cc1cccc(C(=O)Nc2cc3sc([C@H]4CC[C@H](C(=O)N5CCN(CCCNc6cccc7c6C(=O)N(C6CCC(=O)NC6=O)C7=O)CC5)CC4)nc3cc2C(C)(C)O)n1] |
| I-466 | 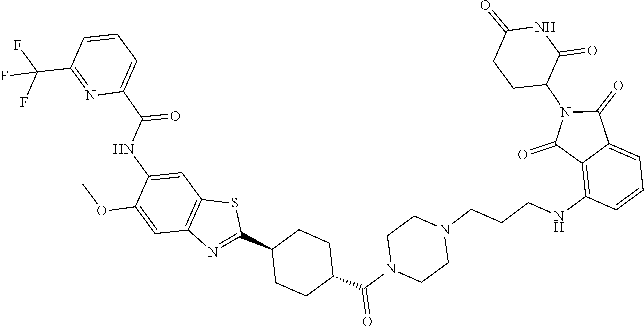 [SMILES: COc1cc2nc([C@H]3CC[C@H](C(=O)N4CCN(CCCNc5cccc6c5C(=O)N(C5CCC(=O)NC5=O)C6=O)CC4)CC3)sc2cc1NC(=O)c1cccc(C(F)(F)F)n1] |
| I-467 | 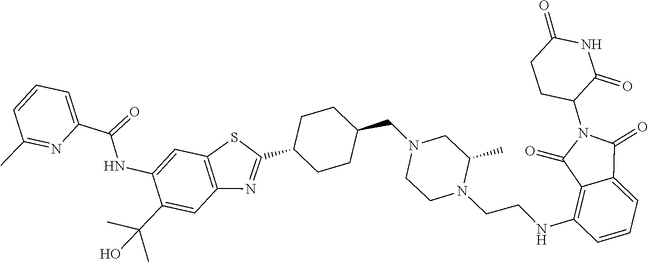 [SMILES: Cc1cccc(C(=O)Nc2cc3sc([C@H]4CC[C@H](CN5CCN(CCNc6cccc7c6C(=O)N(C6CCC(=O)NC6=O)C7=O)[C@@H](C)C5)CC4)nc3cc2C(C)(C)O)n1] |
| I-468 | 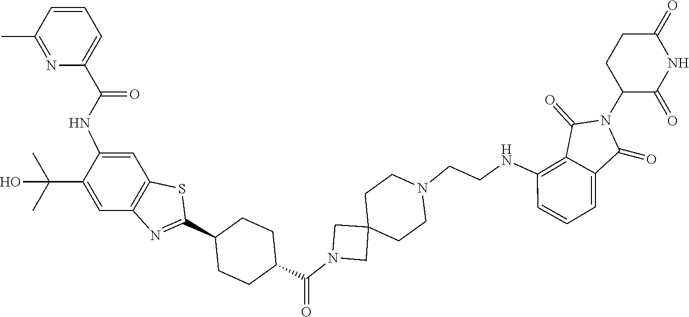 [SMILES: Cc1cccc(C(=O)Nc2cc3sc([C@H]4CC[C@H](C(=O)N5CC6(CCN(CCNc7cccc8c7C(=O)N(C7CCC(=O)NC7=O)C8=O)CC6)C5)CC4)nc3cc2C(C)(C)O)n1] |
| I-469 | 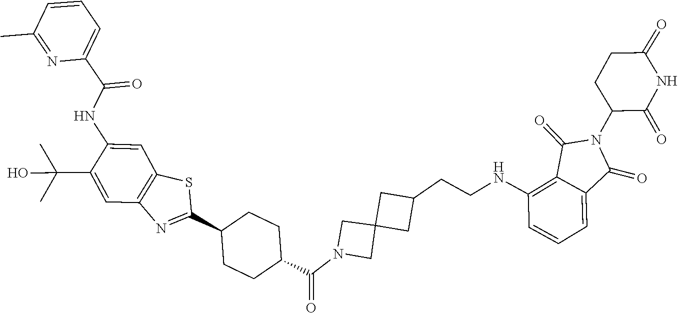 [SMILES: Cc1cccc(C(=O)Nc2cc3sc([C@H]4CC[C@H](C(=O)N5CC6(CC(CCNc7cccc8c7C(=O)N(C7CCC(=O)NC7=O)C8=O)C6)C5)CC4)nc3cc2C(C)(C)O)n1] |
| I-470 | 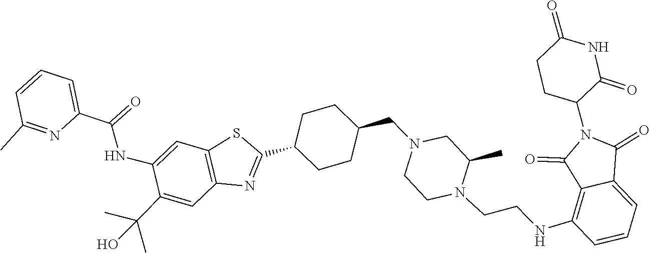 [SMILES: Cc1cccc(C(=O)Nc2cc3sc([C@H]4CC[C@H](CN5CCN(CCNc6cccc7c6C(=O)N(C6CCC(=O)NC6=O)C7=O)[C@H](C)C5)CC4)nc3cc2C(C)(C)O)n1] |
| I-471 | 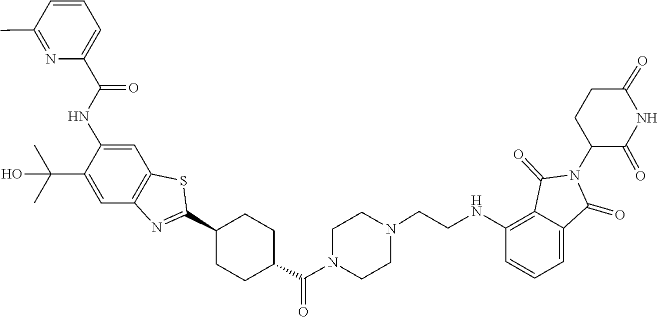 [SMILES: Cc1cccc(C(=O)Nc2cc3sc([C@H]4CC[C@H](C(=O)N5CCN(CCNc6cccc7c6C(=O)N(C6CCC(=O)NC6=O)C7=O)CC5)CC4)nc3cc2C(C)(C)O)n1] |
| I-472 | 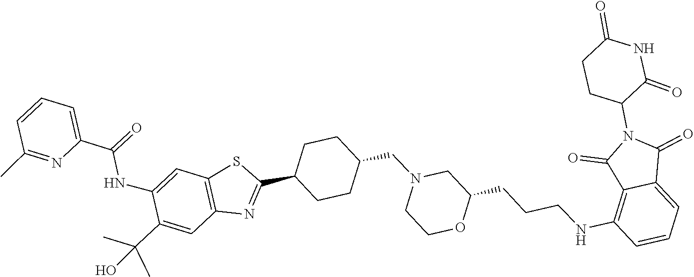 [SMILES: Cc1cccc(C(=O)Nc2cc3sc([C@H]4CC[C@H](CN5CCO[C@@H](CCCNc6cccc7c6C(=O)N(C6CCC(=O)NC6=O)C7=O)C5)CC4)nc3cc2C(C)(C)O)n1] |
| I-473 | 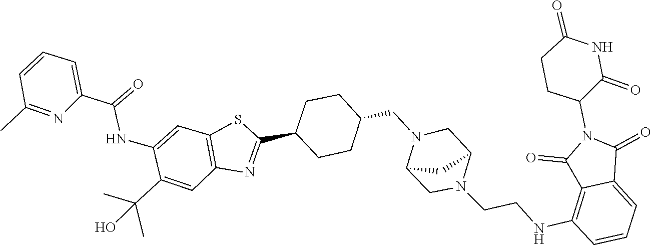 [SMILES: Cc1cccc(C(=O)Nc2cc3sc([C@H]4CC[C@H](CN5C[C@@H]6C[C@H]5CN6CCNc5cccc6c5C(=O)N(C5CCC(=O)NC5=O)C6=O)CC4)nc3cc2C(C)(C)O)n1] |
| I-474 | 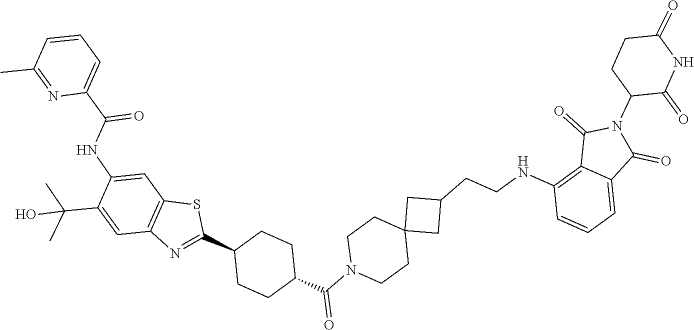 [SMILES: Cc1cccc(C(=O)Nc2cc3sc([C@H]4CC[C@H](C(=O)N5CCC6(CC5)CC(CCNc5cccc7c5C(=O)N(C5CCC(=O)NC5=O)C7=O)C6)CC4)nc3cc2C(C)(C)O)n1] |
| I-475 | 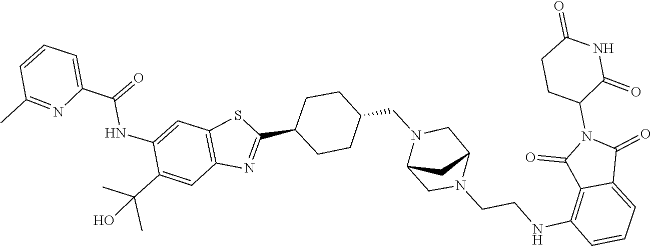 [SMILES: Cc1cccc(C(=O)Nc2cc3sc([C@H]4CC[C@H](CN5C[C@H]6C[C@@H]5CN6CCNc5cccc6c5C(=O)N(C5CCC(=O)NC5=O)C6=O)CC4)nc3cc2C(C)(C)O)n1] |
| I-476 | 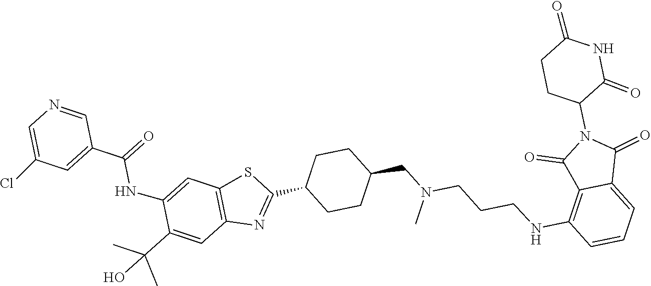 [SMILES: CN(CCCNc1cccc2c1C(=O)N(C1CCC(=O)NC1=O)C2=O)C[C@H]1CC[C@H](c2nc3cc(C(C)(C)O)c(NC(=O)c4cncc(Cl)c4)cc3s2)CC1] |
| I-477 | 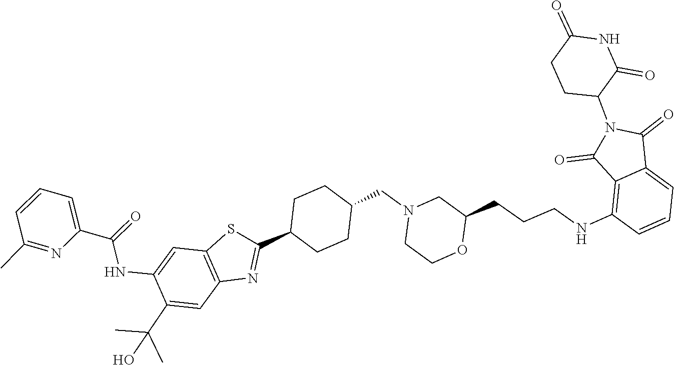 [SMILES: Cc1cccc(C(=O)Nc2cc3sc([C@H]4CC[C@H](CN5CCO[C@H](CCCNc6cccc7c6C(=O)N(C6CCC(=O)NC6=O)C7=O)C5)CC4)nc3cc2C(C)(C)O)n1] |
| I-478 | 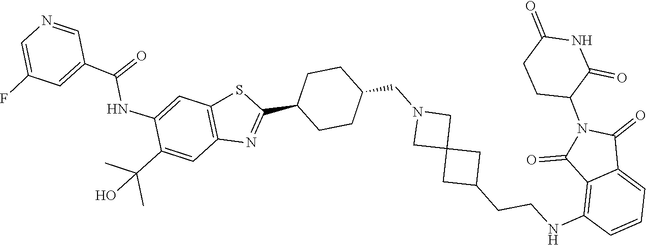 [SMILES: CC(C)(O)c1cc2nc([C@H]3CC[C@H](CN4CC5(CC(CCNc6cccc7c6C(=O)N(C6CCC(=O)NC6=O)C7=O)C5)C4)CC3)sc2cc1NC(=O)c1cncc(F)c1] |
| I-479 | 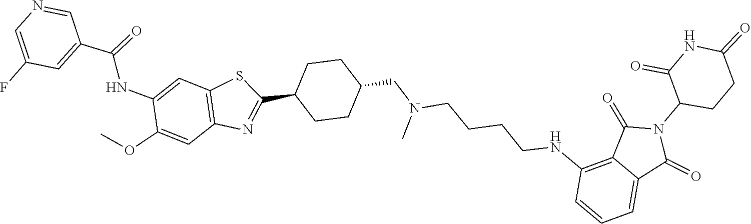 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cncc(F)c1] |
| I-480 | 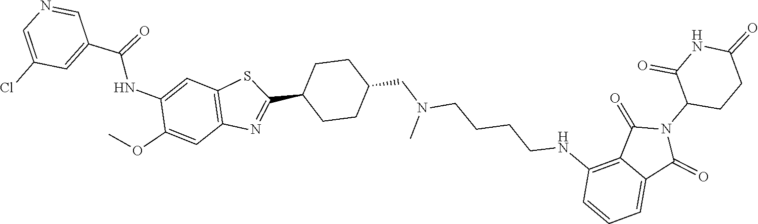 [SMILES: COc1cc2nc([C@H]3CC[C@H](CN(C)CCCCNc4cccc5c4C(=O)N(C4CCC(=O)NC4=O)C5=O)CC3)sc2cc1NC(=O)c1cncc(Cl)c1] |
| I-481 | 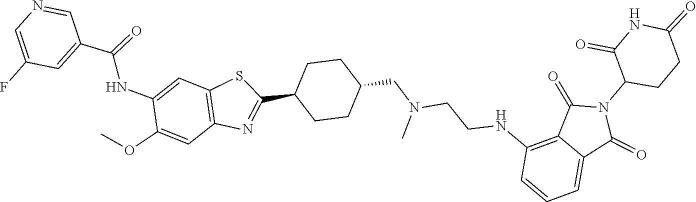 [SMILES: COc1cc2nc([C@H]3CC[C@H](CN(C)CCNc4cccc5c4C(=O)N(C4CCC(=O)NC4=O)C5=O)CC3)sc2cc1NC(=O)c1cncc(F)c1] |
| I-482 | 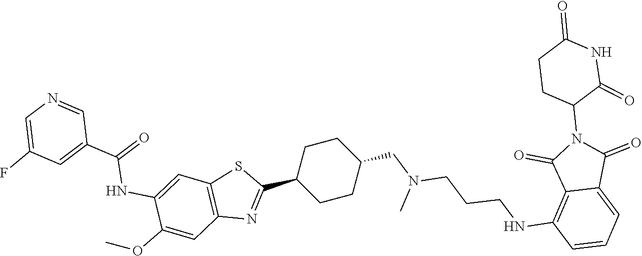 [SMILES: COc1cc2nc([C@H]3CC[C@H](CN(C)CCCNc4cccc5c4C(=O)N(C4CCC(=O)NC4=O)C5=O)CC3)sc2cc1NC(=O)c1cncc(F)c1] |
| I-483 | 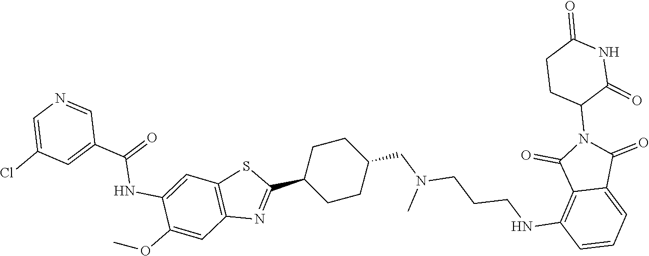 [SMILES: COc1cc2nc([C@H]3CC[C@H](CN(C)CCCNc4cccc5c4C(=O)N(C4CCC(=O)NC4=O)C5=O)CC3)sc2cc1NC(=O)c1cncc(Cl)c1] |
| I-484 | 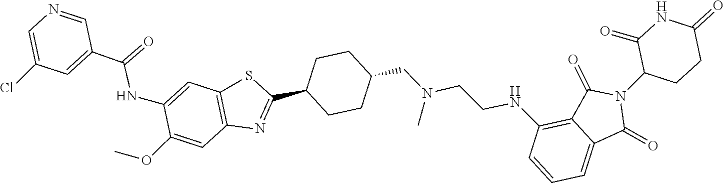 [SMILES: COc1cc2nc([C@H]3CC[C@H](CN(C)CCNc4cccc5c4C(=O)N(C4CCC(=O)NC4=O)C5=O)CC3)sc2cc1NC(=O)c1cncc(Cl)c1] |
| I-485 | 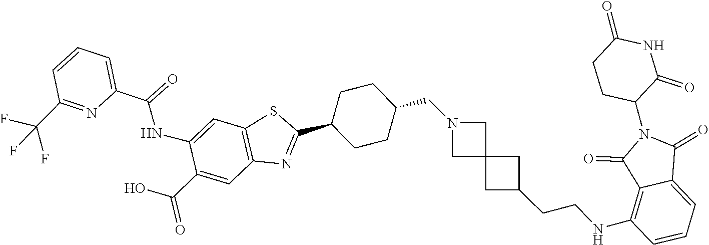 [SMILES: O=C1CCC(N2C(=O)c3cccc(NCCC4CC5(C4)CN(C[C@H]4CC[C@H](c6nc7cc(C(=O)O)c(NC(=O)c8cccc(C(F)(F)F)n8)cc7s6)CC4)C5)c3C2=O)C(=O)N1] |
| I-486 | 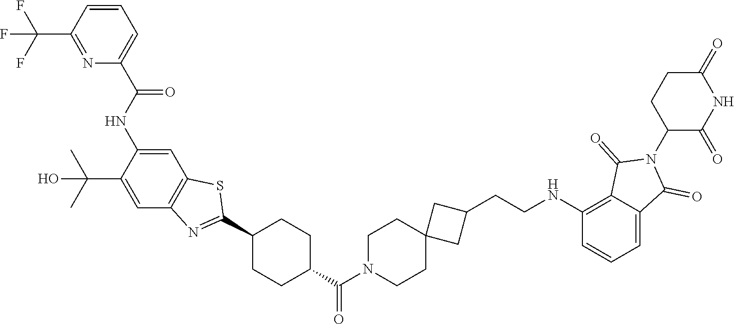 [SMILES: CC(C)(O)c1cc2nc([C@H]3CC[C@H](C(=O)N4CCC5(CC4)CC(CCNc4cccc6c4C(=O)N(C4CCC(=O)NC4=O)C6=O)C5)CC3)sc2cc1NC(=O)c1cccc(C(F)(F)F)n1] |
| I-487 | 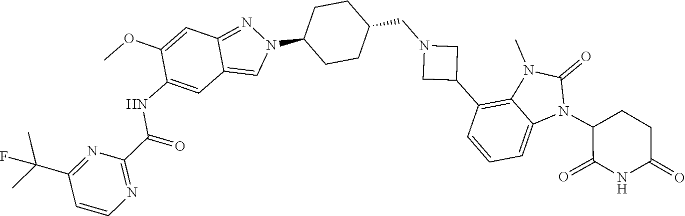 [SMILES: COc1cc2nn([C@H]3CC[C@H](CN4CC(c5cccc6c5n(C)c(=O)n6C5CCC(=O)NC5=O)C4)CC3)cc2cc1NC(=O)c1nccc(C(C)(C)F)n1] |
| I-488 | 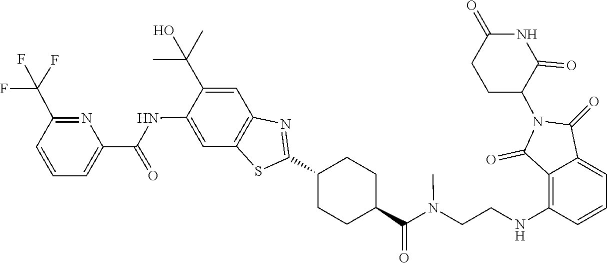 [SMILES: CN(CCNc1cccc2c1C(=O)N(C1CCC(=O)NC1=O)C2=O)C(=O)[C@H]1CC[C@H](c2nc3cc(C(C)(C)O)c(NC(=O)c4cccc(C(F)(F)F)n4)cc3s2)CC1] |
| I-489 | 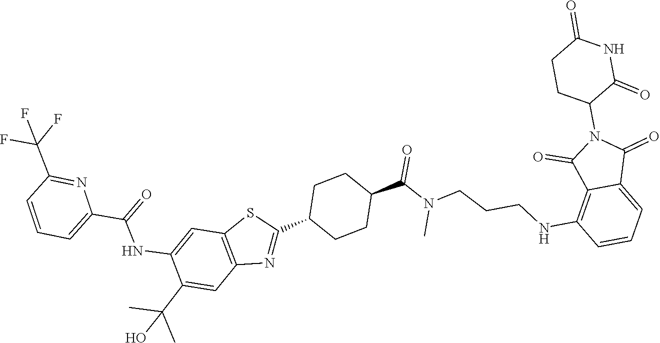 [SMILES: CN(CCCNc1cccc2c1C(=O)N(C1CCC(=O)NC1=O)C2=O)C(=O)[C@H]1CC[C@H](c2nc3cc(C(C)(C)O)c(NC(=O)c4cccc(C(F)(F)F)n4)cc3s2)CC1] |
| I-490 | 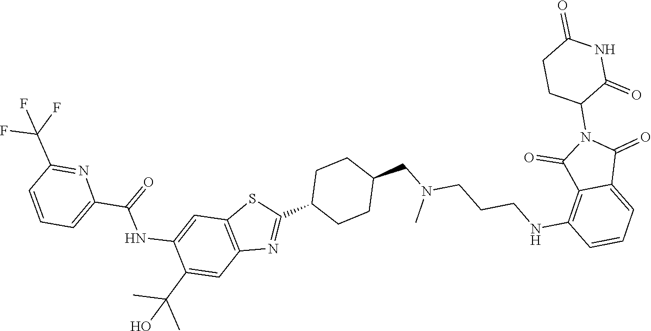 [SMILES: CN(CCCNc1cccc2c1C(=O)N(C1CCC(=O)NC1=O)C2=O)C[C@H]1CC[C@H](c2nc3cc(C(C)(C)O)c(NC(=O)c4cccc(C(F)(F)F)n4)cc3s2)CC1] |
| I-491 | 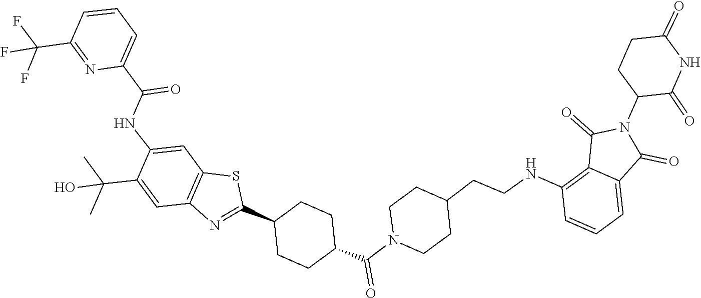 [SMILES: CC(C)(O)c1cc2nc([C@H]3CC[C@H](C(=O)N4CCC(CCNc5cccc6c5C(=O)N(C5CCC(=O)NC5=O)C6=O)CC4)CC3)sc2cc1NC(=O)c1cccc(C(F)(F)F)n1] |
| I-492 | 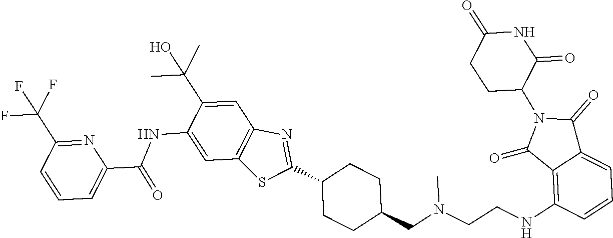 [SMILES: CN(CCNc1cccc2c1C(=O)N(C1CCC(=O)NC1=O)C2=O)C[C@H]1CC[C@H](c2nc3cc(C(C)(C)O)c(NC(=O)c4cccc(C(F)(F)F)n4)cc3s2)CC1] |
| I-493 | 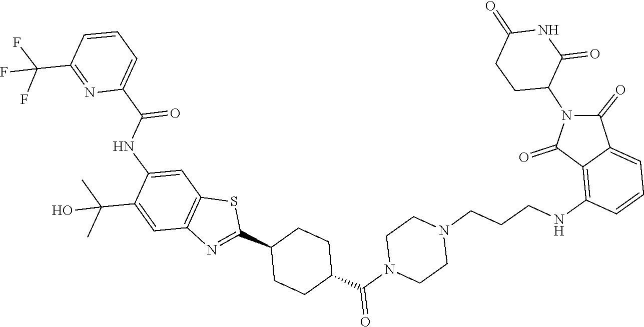 [SMILES: CC(C)(O)c1cc2nc([C@H]3CC[C@H](C(=O)N4CCN(CCCNc5cccc6c5C(=O)N(C5CCC(=O)NC5=O)C6=O)CC4)CC3)sc2cc1NC(=O)c1cccc(C(F)(F)F)n1] |

In some embodiments, the present invention provides a compound set forth in Table 1, above, or a pharmaceutically acceptable salt thereof.

In some embodiments, the present invention provides a compound that is not one or more of the following:

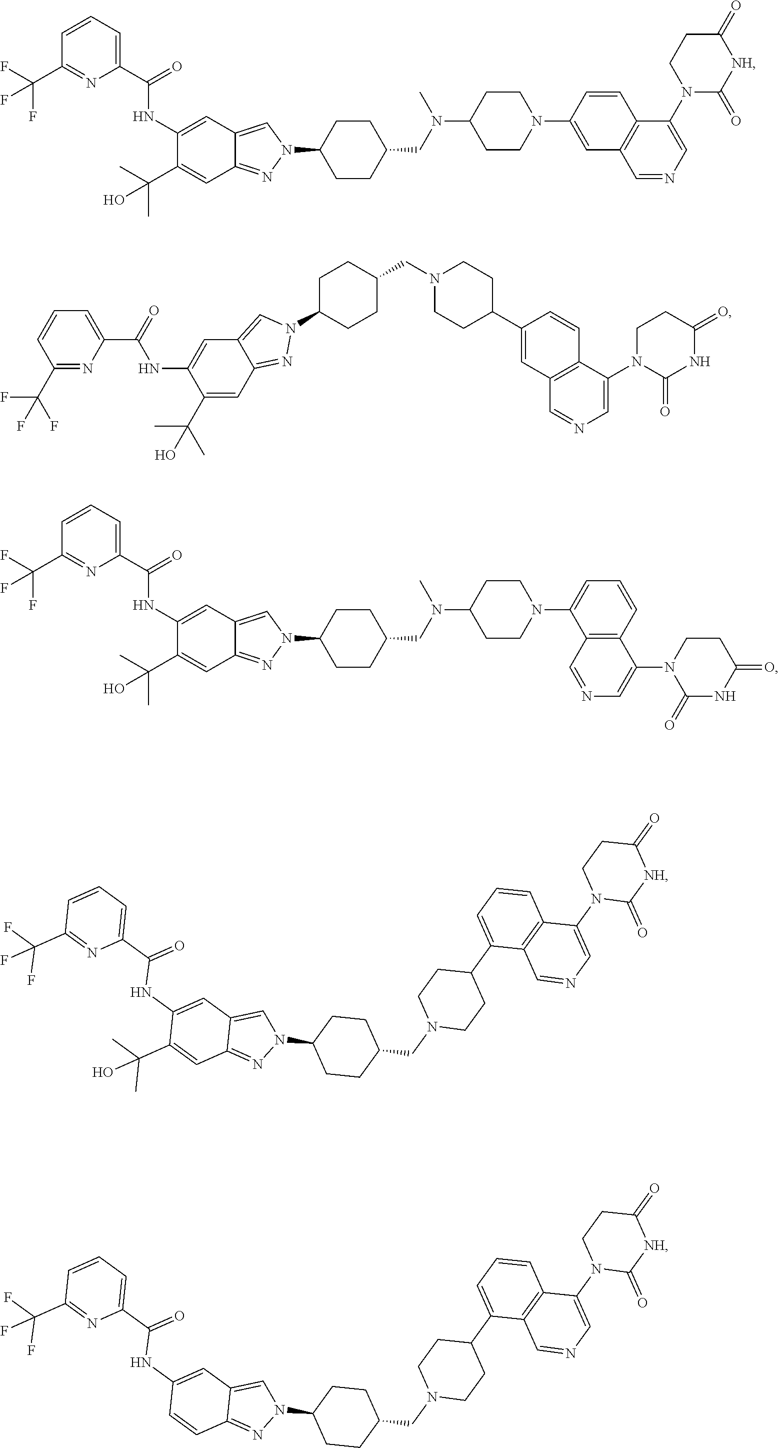

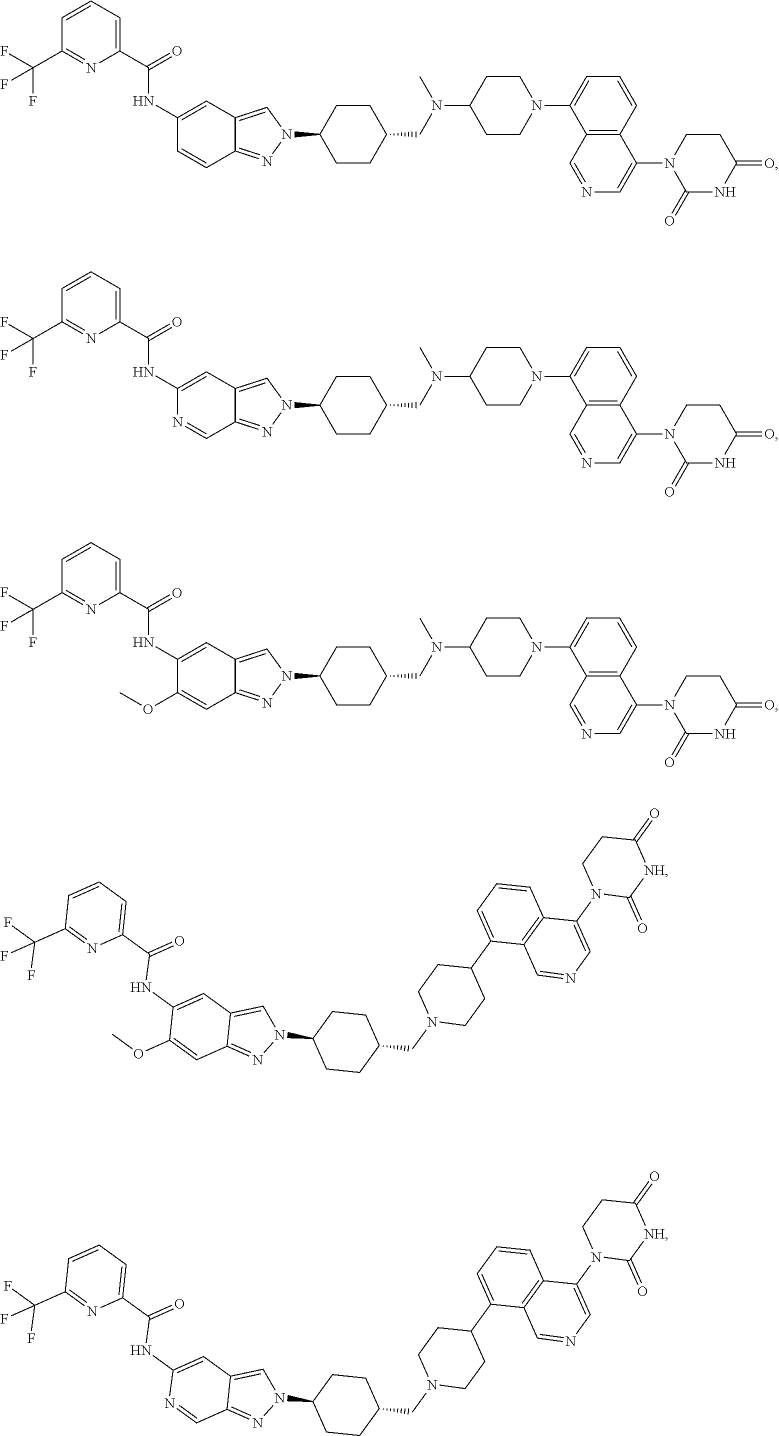

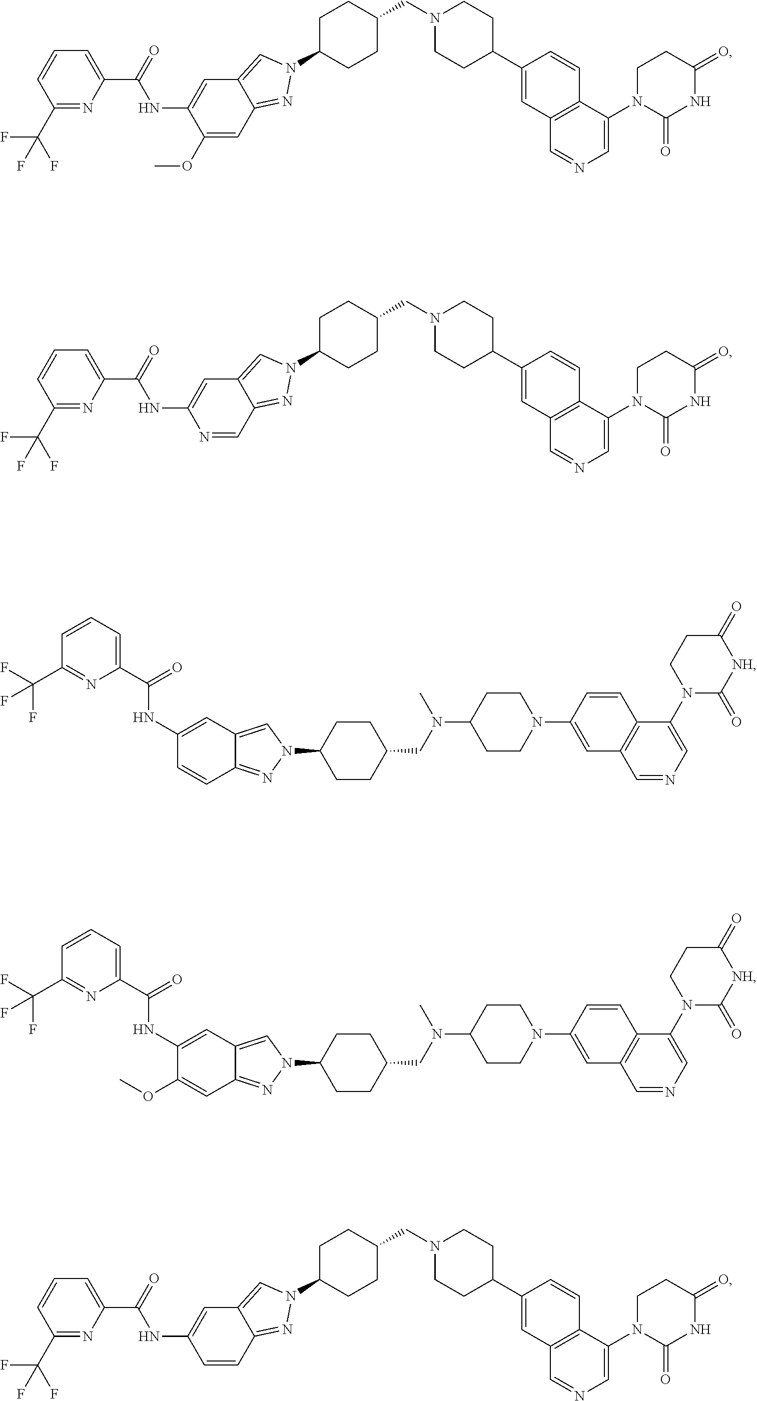

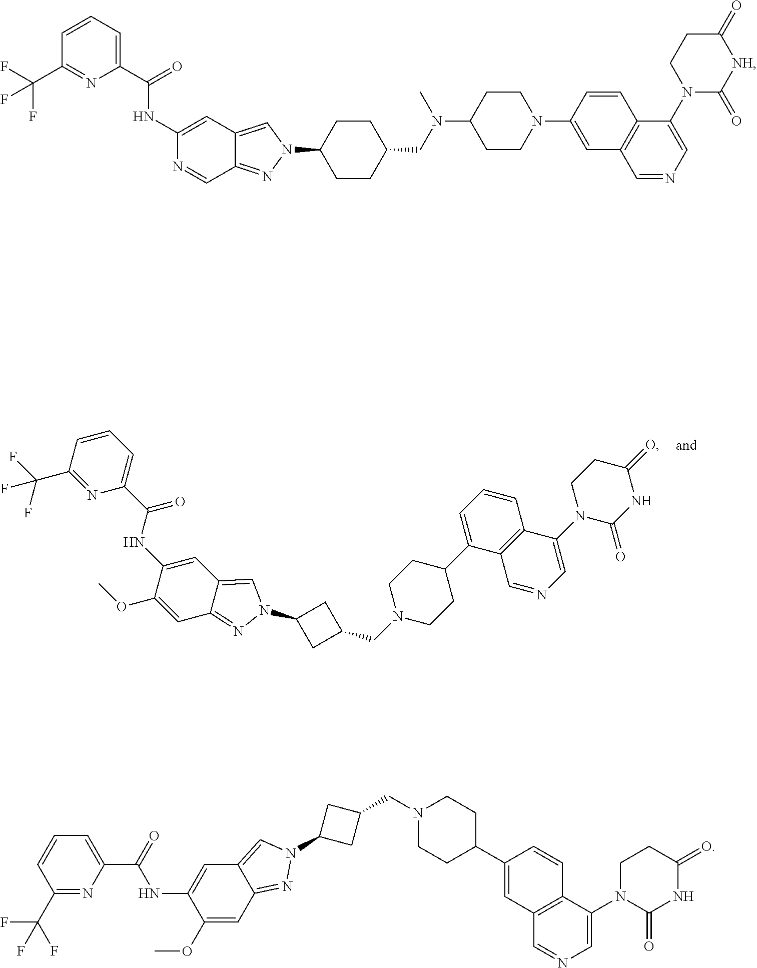 [SMILES: CN(C[C@H]1CC[C@H](n2cc3cc(NC(=O)c4cccc(C(F)(F)F)n4)ncc3n2)CC1)C1CCN(c2ccc3c(N4CCC(=O)NC4=O)cncc3c2)CC1 ; COc1cc2nn([C@H]3C[C@H](CN4CCC(c5cccc6c(N7CCC(=O)NC7=O)cncc56)CC4)C3)cc2cc1NC(=O)c1cccc(C(F)(F)F)n1 ; "and" ; COc1cc2nn([C@H]3C[C@H](CN4CCC(c5ccc6c(N7CCC(=O)NC7=O)cncc6c5)CC4)C3)cc2cc1NC(=O)c1cccc(C(F)(F)F)n1]

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive DIM moiety (e.g., amine, alcohol, etc.), it is not shown but it is generally appreciated and well known by those having ordinary skill in the art that the reactivity of said reactive DIM moiety may be masked by employing a suitable protecting group that can thereafter be removed in situ or during a separate synthetic step.

In certain embodiments, compounds of the present invention are generally prepared according to Scheme 1 set forth below:

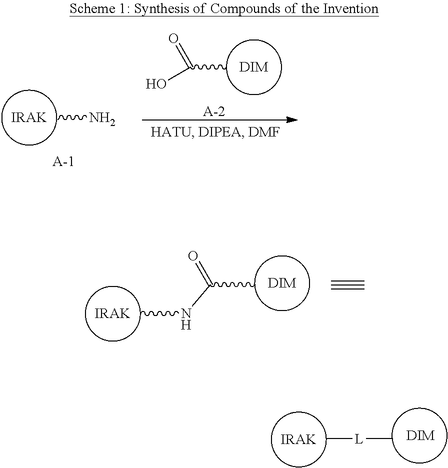

As depicted in Scheme 1, above, amine A-1 is coupled to acid A-2 using the coupling agent HATU in the presence of the base DIPEA in DMF to form a compound of the invention with a linker comprising an amide bond. The squiggly bond, [symbol] , represents the portion of the linker between IRAK and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 2 set forth below:

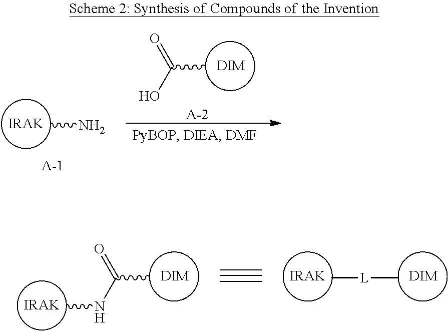

As depicted in Scheme 2, above, amine A-1 is coupled to acid A-2 using the coupling agent PyBOP in the presence of the base DIPEA in DMF to form a compound of the invention with a linker comprising an amide bond. The squiggly bond, [symbol] , represents the portion of the linker between IRAK and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 3 set forth below:

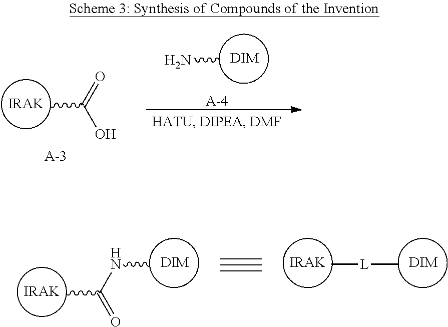

As depicted in Scheme 3, above, acid A-3 is coupled to amine A-4 using the coupling agent HATU in the presence of the base DIPEA in DMF to form a compound of the invention with a linker comprising an amide bond. The squiggly bond, [symbol] , represents the portion of the linker between IRAK and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 4 set forth below:

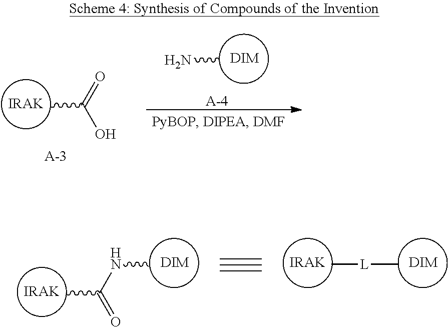

As depicted in Scheme 4, above, acid A-3 is coupled to amine A-4 using the coupling agent PyBOP in the presence of the base DIPEA in DMF to form a compound of the invention with a linker comprising an amide bond. The squiggly bond, [symbol] , represents the portion of the linker between IRAK and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 5 set forth below:

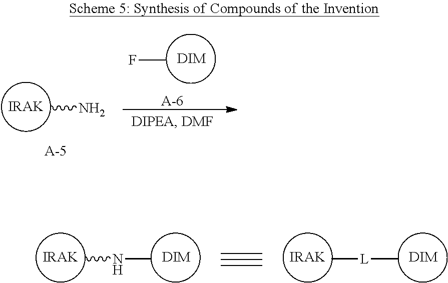

As depicted in Scheme 5, above, an S_{N}Ar displacement of fluoride A-6 by amine A-5 is effected in the presence of the base DIPEA in DMF to form a compound of the invention with a linker comprising a secondary amine. The squiggly bond, [symbol] , represents the portion of the linker between IRAK and the terminal amino group of A-5.

In certain embodiments, compounds of the present invention are generally prepared according to Scheme 6 set forth below:

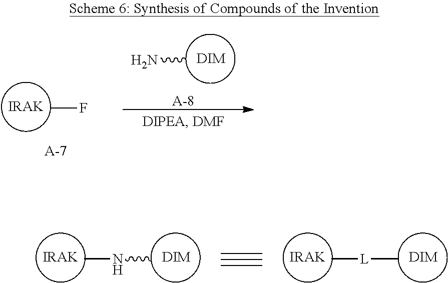

As depicted in Scheme 6, above, an S_{N}Ar displacement of fluoride A-7 by amine A-8 is effected in the presence of the base DIPEA in DMF to form a compound of the invention with a linker comprising a secondary amine. The squiggly bond, [symbol] , represents the portion of the linker between DIM and the terminal amino group of A-8.

In certain embodiments, compounds of the present invention are generally prepared according to Scheme 7 set forth below:

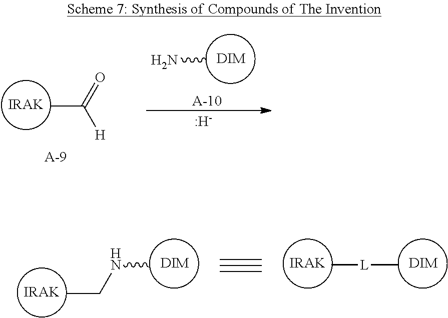

As depicted in Scheme 7, above, reductive alkylation of aldehyde A-9 by amine A-10 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between DIM and the terminal amino group of A-10.

In certain embodiments, compounds of the present invention are generally prepared according to Scheme 8 set forth below:

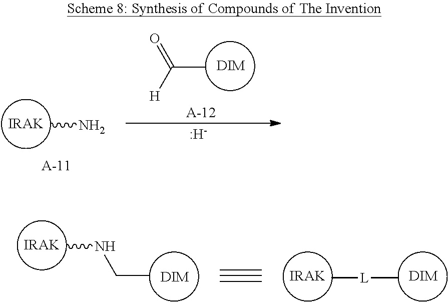

As depicted in Scheme 8, above, reductive alkylation of aldehyde A-12 by amine A-11 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between IRAK and the terminal amino group of A-11.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5^{th} Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing compounds of the invention are described below in the Exemplification.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that it is effective to measurably degrade and/or inhibit an IRAK protein kinase, or a mutant thereof, in a biological sample or in a patient. In certain embodiments, the amount of compound in compositions of this invention is such that it is effective to measurably degrade and/or inhibit an IRAK protein kinase, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of an IRAK protein kinase, or a mutant thereof.

As used herein, the term “degratorily active metabolite or residue thereof” means that a metabolite or residue thereof is also a degrader of an IRAK protein kinase, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation and/or inhibition of kinase activity of one or more enzymes.

Examples of kinases that are degraded and/or inhibited by the compounds and compositions described herein and against which the methods described herein are useful include those of the interleukin-1 receptor-associated kinase (IRAK) family of kinases, the members of which include IRAK-1, IRAK-2, and IRAK-4, or a mutant thereof. Li et al., “IRAK-4: A novel member of the IRAK family with the properties of an IRAK-kinase,” PNAS2002, 99(8), 5567-5572, Flannery et al., “The interleukin-1 receptor-associated kinases: Critical regulators of innate immune signaling” Biochem Pharm 2010, 80(12), 1981-1991 incorporated by reference in its entirety.

The activity of a compound utilized in this invention as a degrader and/or inhibitor of IRAK-1, IRAK-2, and/or IRAK-4, or a mutant thereof, may be assayed in vitro, in vivo or in a cell line. In vitro assays include assays that determine inhibition of either the phosphorylation activity and/or the subsequent functional consequences, or ATPase activity of activated IRAK-1, IRAK-2, and/or IRAK-4, or a mutant thereof. Alternate in vitro assays quantitate the ability of the inhibitor to bind to IRAK-1, IRAK-2 and/or IRAK-4. Inhibitor binding may be measured by radiolabeling the inhibitor prior to binding, isolating the inhibitor/IRAK-1, inhibitor/IRAK-2, or inhibitor/IRAK-4 complex and determining the amount of radiolabel bound. Alternatively, inhibitor binding may be determined by running a competition experiment where new inhibitors are incubated with IRAK-1, IRAK-2, and/or IRAK-4 bound to known radioligands. Representative in vitro and in vivo assays useful in assaying an IRAK-4 inhibitor include those described and disclosed in, e.g., Kim et al., “A critical role for IRAK4 kinase activity in Toll-like receptor-mediated innate immunity,” J. Exp. Med. 2007 204(5), 1025-1036; Lebakken et al., “A Fluorescence Lifetime Based Binding Assay to Characterize Kinase Inhibitors,” J. Biomol. Screen. 2007, 12(6), 828-841; Maschera et al., “Overexpression of an enzymatically inactive interleukin-1-receptor-associated kinase activates nuclear factor-KB,” Biochem. J. 1999, 339, 227-231; Song et al., “The kinase activities of interleukin-e receptor associated kinase (IRAK)-1 and 4 are redundant in the control of inflammatory cytokine expression in human cells,” Mol. Immunol. 2009, 46, 1458-1466, each of, the entirety of each of which is herein incorporated by reference. Detailed conditions for assaying a compound utilized in this invention as a degrader and/or inhibitor of IRAK-1, IRAK-2, and/or IRAK-4, or a mutant thereof, are set forth in the Examples below.

The best characterized member of the IRAK family is the serine/threonine kinase IRAK-4. IRAK-4 is implicated in signaling innate immune responses from Toll-like receptors (TLRs) and Toll/IL-1 receptors (TIRs).

Innate immunity detects pathogens through the recognition of pathogen-associated molecular patterns by TLRs, when then links to the adaptive immune response. TLRs recognize conserved structures of both microbes and endogenous molecules. TLRs which recognize bacterial and fungal components are located on the cell surface, whereas TLRs which recognize viral or microbial nucleic acids are localized to intracellular membranes such as endosomes and phagosomes. Cell surface TLRs can be targeted by small molecules and antibodies, whereas intracellular TLRs require targeting with oligonucleotides.

TLRs mediate the innate immune response by upregulating the expression of inflammatory genes in multiple target cells. See, e.g., Sen et al., “Transcriptional signaling by double-stranded RNA: role of TLR3,” Cytokine & Growth Factor Rev. 2005, 16, 1-14, incorporated by reference in its entirety. While TLR-mediated inflammatory response is critical for innate immunity and host defense against infections, uncontrolled inflammation is detrimental to the host leading to sepsis and chronic inflammatory diseases, such as chronic arthritis, atherosclerosis, multiple sclerosis, cancers, autoimmune disorders such as rheumatoid arthritis, lupus, asthma, psoriasis, and inflammatory bowel diseases.

Upon binding of a ligand, most TLRs recruit the adaptor molecule MyD88 through the TIR domain, mediating the MyD88-dependent pathway. MyD88 then recruits IRAK-4, which engages with the nuclear factor-κB (NF-κB), mitogen-activated protein (MAP) kinase and interferon-regulatory factor cascades and leads to the induction of pro-inflammatory cytokines. The activation of NF-κB results in the induction of inflammatory cytokines and chemokines, such as TNF-α, IL-1 α, IL-6 and IL-8. The kinase activity of IRAK-4 has been shown to play a critical role in the TLR-mediated immune and inflammatory responses. IRAK4 is a key mediator of the innate immune response orchestrated by interleukin-1 receptor (IL-1R), interleukin-18 receptor (IL-18R), IL-33 receptor (IL-33R), and Toll-like receptors (TLRs). Inactivation of IRAK-1 and/or IRAK-4 activity has been shown to result in diminished production of cytokines and chemokines in response to stimulation of IL-1 and TLR ligands. See, e.g., Picard et al., “Clinical features and outcome of patients with IRAK-4 and MyD88 deficiency,” Medicine (Baltimore), 2010, 89(6), 043-25; Li, “IRAK4 in TLR/IL-1R signaling: Possible clinical applications,” Eur J. Immunology 2008, 38:614-618; Cohen et al., “Targeting protein kinases for the development of anti-inflammatory drugs,” Curr Opin. Cell Bio. 2009, 21:317-324; Flannery et al., “The interleukin-1 receptor-associated kinases: Critical regulators of innate immune signalling,” Biochem. Pharm. 2010, 80(12), 1981-1991; Gottipati et al., “IRAK1: A critical signaling mediator of innate immunity,” Cellular Signaling 2008, 20, 269-276; Kim et al., “A critical role for IRAK4 kinase activity in Toll-like receptor-mediated innate immunity,” J. Exp. Med. 2007 204(5), 1025-1036; Koziczak-Holbro et al., “IRAK-4 Kinase Activity Is Required for Interleukin-1 (IL-1) Receptor- and Toll-like Receptor 7-mediated Signaling and Gene Expression,” J. Biol. Chem. 2007, 282(18), 13552-13560; Kubo-Murai et al., “IRAK-4-dependent Degradation of IRAK-1 is a Negative Feedback Signal for TLR-mediated NF-κB Activation,” J. Biochem. 2008, 143, 295-302; Maschera et al., “Overexpression of an enzymatically inactive interleukin-1-receptor-associated kinase activates nuclear factor-KB,” Biochem. J. 1999, 339, 227-231; Lin et al., “Helical assembly in the MyD88-IRAK4-IRAK2 complex in TLR/IL-1R signalling,” Nature 2010, 465(17), 885-891; Suzuki et al., “IRAK-4 as the central TIR signaling mediator in innate immunity,” TRENDS in Immunol. 2002, 23(10), 503-506; Suzuki et al., “Severe impairment of interleukin-1 and Toll-like receptor signalling in mice lacking IRAK-4,” Nature 2002, 416, 750-754; Swantek et al., “IL-1 Receptor-Associated Kinase Modulates Host Responsiveness to Endotoxin,” J. Immunol. 2000, 164, 4301-4306; Hennessy, E., et al., “Targeting Toll-like receptors: emerging therapeutics?” Nature Reviews, vol. 9, pp: 293-307 (2010); Dinarello, C. “Interleukin-18 and the Pathogenesis of Inflammatory Diseases,” Seminars in Nephrology, vol. 27, no. 1, pp: 98-114 (2007), each of, the entirety of each of which is herein incorporated by reference. In fact, knockdown mice that express a catalytically inactive mutant IRAK-4 protein are completely resistant to septic shock and show impaired IL-1 activity. Moreover, these mice are resistant to joint and bone inflammation/destruction in an arthritis model, suggesting that IRAK-4 may be targeted to treat chronic inflammation. Further, while IRAK-4 appears to be vital for childhood immunity against some pyogenic bacteria, it has been shown to play a redundant role in protective immunity to most infections in adults, as demonstrated by one study in which patients older than 14 lacking IRAK-4 activity exhibited no invasive infections. Cohen et al., “Targeting protein kinases for the development of anti-inflammatory drugs,” Curr Opin. Cell Bio. 2009, 21:317-324; Ku et al., “Selective predisposition to bacterial infections in IRAK-4-deficient children: IRAK-4-dependent TLRs are otherwise redundant in protective immunity,” J. Exp. Med. 2007, 204(10), 2407-2422; Picard et al., “Inherited human IRAK-4 deficiency: an update,” Immunol. Res. 2007, 38, 347-352; Song et al., “The kinase activities of interleukin-e receptor associated kinase (IRAK)-1 and 4 are redundant in the control of inflammatory cytokine expression in human cells,” Mot Immunol. 2009, 46, 1458-1466; Rokosz, L. et al., “Kinase inhibitors as drugs for chronic inflammatory and immunological diseases: progress and challenges,” Expert Opinions on Therapeutic Targets, 12(7), pp: 883-903 (2008); Gearing, A. “Targeting toll-like receptors for drug development: a summary of commercial approaches,” Immunology and Cell Biology, 85, pp: 490-494 (2007); Dinarello, C. “IL-1: Discoveries, controversies and future directions,” European Journal of Immunology, 40, pp: 595-653 (2010), each of, the entirety of each of which is herein incorporated by reference. Because TLR activation triggers IRAK-4 kinase activity, IRAK-4 inhibition presents an attractive target for treating the underlying causes of inflammation in countless diseases.

Representative IRAK-4 inhibitors include those described and disclosed in e.g., Buckley et al., Bioorg. Med. Chem. Lett. 2008, 18, 3211-3214; Buckley et al., Bioorg. Med. Chem. Lett. 2008, 18, 3291-3295; Buckley et al., Bioorg. Med. Chem. Lett. 2008, 18, 3656-3660; Powers et al., “Discovery and initial SAR of inhibitors of interleukin-1 receptor-associated kinase-4,” Bioorg. Med. Chem. Lett. 2006, 16, 2842-2845; Wang et al., “IRAK-4 Inhibitors for Inflammation,” Curr Topics in Med. Chem. 2009, 9, 724-737, each of, the entirety of each of which is herein incorporated by reference.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of one of more of IRAK-1, IRAK-2, and/or IRAK-4 and are therefore useful for treating one or more disorders associated with activity of one or more of IRAK-1, IRAK-2, and/or IRAK-4. Thus, in certain embodiments, the present invention provides a method for treating a IRAK-1-mediated, a IRAK-2-mediated, and/or a IRAK-4-mediated disorder comprising the step of administering to a patient in need thereof a compound of the present invention, or pharmaceutically acceptable composition thereof.

As used herein, the terms “IRAK-1-mediated”, “IRAK-2-mediated”, and/or “IRAK-4-mediated” disorders, diseases, and/or conditions as used herein means any disease or other deleterious condition in which one or more of IRAK-1, IRAK-2, and/or IRAK-4, or a mutant thereof, are known to play a role. Accordingly, another embodiment of the present invention relates to treating or lessening the severity of one or more diseases in which one or more of IRAK-1, IRAK-2, and/or IRAK-4, or a mutant thereof, are known to play a role.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

Diseases and conditions treatable according to the methods of this invention include, but are not limited to, cancer (see, e.g., Ngo, V. et al., “Oncogenically active MYD88 mutations in human lymphoma,” Nature, vol. 000, pp: 1-7 (2010); Lust, J. et al., “Induction of a Chronic Disease State in patients With Smoldering of Indolent Multiple Myeloma by Targeting Interleukin 1β-Induced Interleukin 6 Production and the Myeloma Proliferative Component,” Mayo Clinic Proceedings, 84(2), pp: 114-122 (2009)), diabetes, cardiovascular disease, viral disease, autoimmune diseases such as lupus (see, e.g., Dinarello, C. “Interleukin-18 and the Pathogenesis of Inflammatory Diseases,” Seminars in Nephrology, vol. 27, no. 1, pp: 98-114 (2007); Cohen et al., “Targeting protein kinases for the development of anti-inflammatory drugs,” Curr Opin. Cell Bio. 2009, 21:317-324) and rheumatoid arthritis (see, e.g., Geyer, M. et al., “Actual status of antiinterleukin-1 therapies in rheumatic diseases,” Current Opinion in Rheumatology, 22, pp: 246-251 (2010)), auto-inflammatory syndromes (see, e.g., Hoffman, H. et al., “Efficacy and Safety of Rilonacept (Interleukin-1 Trap) in Patients with Cryopyrin-Associated Periodic Syndromes,” Arthritis & Rheumatism, vol. 58, no. 8, pp: 2443-2452 (2008)), atherosclerosis, psoriasis, allergic disorders, inflammatory bowel disease (see, e.g., Cario, E. “Therapeutic Impact of Toll-like Receptors on Inflammatory Bowel Diseases: A Multiple-edged Sword,” Inflamm. Bowel Dis., 14, pp: 411-421 (2008)), inflammation (see, e.g., Dinarello, C. “Interleukin 1 and interleukin 18 as mediators of inflammation and the aging process,” The American Journal of Clinical Nutrition, 83, pp: 447S-455S (2006)), acute and chronic gout and gouty arthritis (see, e.g., Terkeltaub, R. “Update on gout: new therapeutic strategies and options,” Nature, vol. 6, pp: 30-38 (2010); Weaver, A. “Epidemiology of gout,” Cleveland Clinic Journal of Medicine, vol. 75, suppl. 5, pp: S9-S12 (2008); Dalbeth, N. et al., “Hyperuricaemia and gout: state of the art and future perspectives,” Annals of Rheumatic Diseases, 69, pp: 1738-1743 (2010); Martinon, F. et al., “Gout-associated uric acid crystals activate the NALP3 inflammasome,” Nature, vol. 440, pp: 237-241 (2006); So, A. et al., “A pilot study of IL-1 inhibition by anakinra in acute gout,” Arthritis Research & Therapy, vol. 9, no. 2, pp: 1-6 (2007); Terkeltaub, R. et al., “The interleukin 1 inhibitor rilonacept in treatment of chronic gouty arthritis: results of a placebo-controlled, monosequence crossover, non-randomized, single-blind pilot study,” Annals of Rheumatic Diseases, 68, pp: 1613-1617 (2009); Torres, R. et al., “Hyperalgesia, synovitis and multiple biomarkers of inflammation are suppressed by interleukin 1 inhibition in a novel animal model of gouty arthritis,” Annals of Rheumatic Diseases, 68, pp: 1602-1608 (2009)), neurological disorders, metabolic syndrome (see, e.g., Troseid, M. “The role of interleukin-18 in the metabolic syndrome,” Cardiovascular Diabetology, 9:11, pp: 1-8 (2010)), immunodeficiency disorders such as AIDS and HIV (see, e.g., Iannello, A. et al., “Role of Interleukin-18 in the Development and Pathogenesis of AIDS,” AIDS Reviews, 11, pp: 115-125 (2009)), destructive bone disorders (see, e.g., Hennessy, E., et al., “Targeting Toll-like receptors: emerging therapeutics?” Nature Reviews, vol. 9, pp: 293-307 (2010)), osteoarthritis, proliferative disorders, Waldenstrom's Macroglobulinemia (see, e.g., Treon, et al., “Whole genome sequencing reveals a widely expressed mutation (MYD88 L265P) with oncogenic activity in Waldenstrom's Macroglobulinemia” 53^{rd} ASH Annual Meeting; Xu, et al., “A somatic variant in MYD88 (L256P) revealed by whole genome sequencing differentiates lymphoplasmacytic lymphoma from marginal zone lymphomas” 53^{rd} ASH Annual Meeting; Yang et al., “Disruption of MYD88 pathway signaling leads to loss of constitutive IRAK1, NK-κB and JAK/STAT signaling and induces apoptosis of cells expressing the MYD88 L265P mutation in Waldenstrom's Macroglobulinemia” 53^{rd} ASH Annual Meeting; Iriyama et al., “Clinical significance of genetic mutations of CD79B, CARD11, MYD88, and EZH2 genes in diffuse large B-cell lymphoma patients” 53^{rd} ASH Annual Meeting; infectious diseases, conditions associated with cell death, pathologic immune conditions involving T cell activation, and CNS disorders in a patient. In one embodiment, a human patient is treated with a compound of the current invention and a pharmaceutically acceptable carrier, adjuvant, or vehicle, wherein said compound is present in an amount to measurably degrade and/or inhibit IRAK-1 only, IRAK-2-only, IRAK-4-only and/or IRAK1 and IRAK4 kinase activity.

Compounds of the current invention are useful in the treatment of a proliferative disease selected from a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments the proliferative disease which can be treated according to the methods of this invention is an MyD88 driven disorder. In some embodiments, the MyD88 driven disorder which can be treated according to the methods of this invention is selected from ABC DLBCL, Waldenstrom's macroglobulinemia, Hodgkin's lymphoma, primary cutaneous T-cell lymphoma and chronic lymphocytic leukemia.

In some embodiments the proliferative disease which can be treated according to the methods of this invention is an IL-1 driven disorder. In some embodiments the IL-1 driven disorder is Smoldering of indolent multiple myeloma.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds according to the invention are useful in the treatment of heteroimmune diseases. Examples of such heteroimmune diseases include, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example anti-inflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g. between the hours of about 4 to 6 am, i.e. at a time normally substantially distant form any previously administered symptomatic asthma therapy.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil- related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Chuig-Strauss syndrome), eosinophilic granuloma and eosinophil-related disorders affecting the airways occasioned by drug-reaction.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is an disease of the skin. In some embodiments, the inflammatory disease of the skin is selected from contact dermatitis, atopic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, systemic juvenile idiopathic arthritis (SJIA), Cryopyrin Associated Periodic Syndrome (CAPS), and osteoarthritis.

In some embodiments the inflammatory disease which can be treated according to the methods of this invention is a TH17 mediated disease. In some embodiments the TH17 mediated disease is selected from systemic lupus erythematosus, multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Cardiovascular diseases which can be treated according to the methods of this invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the present invention provides a method of treating hidradenitis suppurativa in a patient in need thereof, comprising administering a compound of the present invention, or a pharmaceutically acceptable salt thereof.

In some embodiments, the present invention provides a method of treating atopic dermatitis in a patient in need thereof, comprising administering a compound of the present invention, or a pharmaceutically acceptable salt thereof.

In some embodiments, the present invention provides a method of treating rheumatoid arthritis in a patient in need thereof, comprising administering a compound of the present invention, or a pharmaceutically acceptable salt thereof.

In some embodiments, the present invention provides a method of treating solid and liquid tumors in a patient in need thereof, comprising administering a compound of the present invention, or a pharmaceutically acceptable salt thereof.

In some embodiments, the neurodegenerative disease which can be treated according to the methods of this invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

The loss of IRAK4 function results in decreased Aβ levels in an in vivo murine model of Alzheimer's disease and was associated with diminished microgliosis and astrogliosis in aged mice. Analysis of microglia isolated from the adult mouse brain revealed an altered pattern of gene expression associated with changes in microglial phenotype that were associated with expression of IRF transcription factors that govern microglial phenotype. Further, loss of IRAK4 function also promoted amyloid clearance mechanisms, including elevated expression of insulin-degrading enzyme. Finally, blocking IRAK function restored olfactory behavior (Cameron et al. “Loss of Interleukin Receptor-Associated Kinase 4 Signaling Suppresses Amyloid Pathology and Alters Microglial Phenotype in a Mouse Model of Alzheimer's Disease” Journal of Neuroscience (2012) 32(43), 15112-15123.

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV infection.

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

### Multiple Degradation

In some embodiments, the invention provides compounds that modulate targeted ubiquitination and degradation of one or more IRAK kinase. In some embodiments, a provided compound modulates targeted ubiquitination and degradation of one or more IRAK kinase and one or more additional protein. In some instances, a provided compound modulates targeted ubiquitination and degradation of IRAK4 and one, two, three, four, or five additional proteins.

In certain embodiments, the invention provides compounds that are triple degraders. In certain embodiments, the invention provides compounds that combine IRAK kinase degradation with IKZF1 and IKZF3 degradation. Some of the most commonly employed E3 ligase ligands are thalidomide and its derivatives, lenalidomide and pomalidomide, commonly referred to as IMiDs (immunomodulatory imide drugs). These agents are small-molecule ligands of cereblon (CRBN) (Ito et al. “Identification of a primary target of thalidomide teratogenicity” Science 2010, 327(5971):1345-1350), a substrate adaptor for the ubiquitously expressed cullin ring ligase 4 (CUL4)-RBX1-DDB1-CRBN (CUL4CRBN) E3 ligase. It has been shown that thalidomide interacts with CRBN to form a novel surface, resulting in interactions with neosubstrates such as Ikaros (IKZF1) and Aiolos (IKZF3) and their ubiquitination and subsequent proteasomal degradation (Krönke et al. “Lenalidomide causes selective degradation of IKZF1 and IKZF3 in multiple myeloma cells” Science 2014, 343(6168):301-305; and Lu et al. “The myeloma drug lenalidomide promotes the cereblon-dependent destruction of Ikaros proteins” Science, 2014; 343(6168):305-309). This activity alone has potent antitumor effects in some liquid malignancies, and lenalidomide (Revlimid®) is US Food and Drug Administration approved for the treatment of MCL, multiple myeloma, and myelodysplastic syndromes with deletion of chromosome 5q. Lenalidomide is also undergoing late-stage clinical trials for a number of lymphomas, including MCL and the activated B-cell subtype of diffuse large B-cell lymphoma (ABC DLBCL).

In some instances, degradation of IRAK4 alone is not sufficient to kill the MYD88 L265P mutant DLBCL cell line OCI-LY10 either in vitro or as a flank xenograft in vivo. In some embodiments, a non-IMiD-based degraders effects IRAK degradation in MYD88 mutant ABC DLBCL cell line tumor xenografts but without causing regression. This is consistent with literature demonstrating no effect on growth of OCI-LY10 or other MYD88 mutant lines when the gene encoding IRAK4 is removed at the DNA level using CRISPR/Cas9 editing (Phelan et al. “A multiprotein supercomplex controlling oncogenic signaling in lymphoma” Nature, 2018, 7718:387-391).

It has been shown that activating MYD88 mutations increase production of beta-IFN, a pro-apoptotic cytokine, in ABC-DLBCL cells (Yang et al. “Exploiting synthetic lethality for the therapy of ABC diffuse large B cell lymphoma” Cancer Cell 2012, 21(6):723-737). The cells are rendered resistant to this effect by a concomitant MYD88-driven activation of NFkB signaling via IRF4 and SPIB transactivating CARD11 (Yang, Cancer Cell 2012). IMiDs are also known to increase the IFN response in MYD88 mutant ABC-DLBCL to levels sufficient to increase apoptosis (Yang, Cancer Cell 2012; and Hagner et al. “CC-122, a pleiotropic pathway modifier, mimics an interferon response and has antitumor activity in DLBCL” Blood 2015, 126:779-789). This effect has been shown to synergize with inhibition of NFkB signaling to further drive DLBCL cell death (Yang, Cancer Cell 2012).

In some instances, the combination of an IMiD with a small molecule IRAK4 kinase inhibitor shows little to no additive effect on viability of the MYD88 mutant ABC DLBCL cell lines, such as OCI-LY10. In some embodiments, the combination of an IRAK4 inhibitor with IMiD is less active than an all-in-one IMiD-based IRAK4 degrader.

In certain embodiments, the combination of IRAK kinase degradation with IKZF1 and IKZF3 degradation in an all-in-one IMiD-based IRAK4 degrader shows potent, single agent activity versus MYD88 mutant ABC DLBCL cell lines in vitro and OCI-LY10 xenograft in vivo. In some embodiments, an all-in-one combination of an IMiD-based CRBN-binder and an IRAK4 binding moiety yields IRAK4 degraders that retain degradation of Ikaros (IKZF1) and other known IMiDs neosubstrates, while more strongly inducing an interferon response compared to pomalidomide alone. In some embodiments, IMiD-based IRAK4 degraders are potent at killing MYD88 mutant ABD-DLBCL cell lines in vitro, demonstrating increased activity versus that obtained from combining an IRAK4 inhibitor with IMiDs as single agents.

In certain embodiments, a provided compound comprising an IMiD-based E3 ligase degrades IRAK4, Ikaros, and Aiolos in MYD88 mutant ABC DLBCL cell line xenografts in vivo, and strongly induces a signature of interferon-driven proteins exemplified by IFITI (interferon-inducible transcript 1) and IFIT3 (interferon-inducible transcript 3). In some embodiments, a provided compound comprising an IMiD-based E3 ligase drives regression of tumor xenographs as a single agent.

In some embodiments, the provided compounds of present invention highlight a synergy obtained by combining IRAK4 degradation with IMiD induction of interferon response to drive single agent anti-tumor activity in MYD88 mutant DLBCL and possibly in other heme malignancies. In certain embodiments, a provided compound comprising an IMiD-based E3 ligase degrade IRAK4, Ikaros, and Aiolos acts synergistically. In some embodiments, a provided compound comprising an IRAK4 binder and an IMiD-based E3 ligase degrades IRAK4, Ikaros, and Aiolos with increased activity in comparison to a provided compound comprising the same IRAK4 binder and a non-IMiD-based E3 ligase and the same IMiD-based E3 ligase as a single agent.

In some embodiments, the present invention provides a method of treating MYD88-mutant Waldenstrom macroglobulinemia in a patient in need thereof, comprising administering a compound of the present invention, or a pharmaceutically acceptable salt thereof.

In some embodiments, the present invention provides a method of treating a AML, or a subset thereof, in a patient in need thereof, comprising administering a compound of the present invention, or a pharmaceutically acceptable salt thereof.

In some embodiments, the present invention provides a method of treating NSCLC in a patient in need thereof, comprising administering a compound of the present invention, or a pharmaceutically acceptable salt thereof.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound or a pharmaceutically acceptable salt thereof and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound or a pharmaceutically acceptable salt thereof and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound or a pharmaceutically acceptable salt thereof and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound or a pharmaceutically acceptable salt thereof and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound or a pharmaceutically acceptable salt thereof and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound or a pharmaceutically acceptable salt thereof and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound or a pharmaceutically acceptable salt thereof and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound or a pharmaceutically acceptable salt thereof and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound or a pharmaceutically acceptable salt thereof and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound or a pharmaceutically acceptable salt thereof and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In some embodiments, the present invention provides a method of treating DLBCL comprising administering to a patient in need thereof a provided compound or a pharmaceutically acceptable salt thereof and a CHOP (cyclophosphamide, Hydrodaunorubicin®, Oncovin®, and prednisone or prednisolone) or R-CHOP (rituximab, cyclophosphamide, Hydrodaunorubicin®, Oncovin®, and prednisone or prednisolone) chemotherapy regimen.

In some embodiments, the present invention provides a method of treating DLBCL comprising administering to a patient in need thereof a provided compound or a pharmaceutically acceptable salt thereof and a rituximab/bendamustine chemotherapy regimen.

In some embodiments, the present invention provides a method of treating DLBCL comprising administering to a patient in need thereof a provided compound or a pharmaceutically acceptable salt thereof and a BTK inhibitor (e.g., ibrutinib).

In some embodiments, the present invention provides a method of treating DLBCL comprising administering to a patient in need thereof a provided compound or a pharmaceutically acceptable salt thereof and an anti-CD20 antibody (e.g., rituximab).

In some embodiments, the present invention provides a method of treating DLBCL comprising administering to a patient in need thereof a provided compound or a pharmaceutically acceptable salt thereof and an anti-CD79B ADC (e.g., polatuzumab).

In some embodiments, the present invention provides a method of treating DLBCL comprising administering to a patient in need thereof a provided compound or a pharmaceutically acceptable salt thereof and a BCL2 inhibitor (e.g., venetoclax).

In some embodiments, the present invention provides a method of treating DLBCL comprising administering to a patient in need thereof a provided compound or a pharmaceutically acceptable salt thereof and lenalidomide

In some embodiments, the present invention provides a method of treating DLBCL comprising administering to a patient in need thereof a provided compound or a pharmaceutically acceptable salt thereof and a PI3K inhibitor (e.g., umbralisib).

In some embodiments, the present invention provides a method of treating a T-cell disease or deficiency describing herein comprising administering to a patient in need thereof a provided compound or a pharmaceutically acceptable salt thereof and a PI3K inhibitor (e.g., umbralisib).

In some embodiments, the present invention provides a method of treating DLBCL comprising administering to a patient in need thereof a provided compound or a pharmaceutically acceptable salt thereof and a proteasome inhibitor (e.g., bortezomib)

In some embodiments, the present invention provides a method of treating a T-cell disease or deficiency describing herein comprising administering to a patient in need thereof a provided compound or a pharmaceutically acceptable salt thereof and a protesome inhibitor (e.g., bortezomib).

In another embodiment, the present invention provides a method of treating multiple myeloma comprising administering to a patient in need thereof a provided compound or a pharmaceutically acceptable salt thereof and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenstrom's macroglobulinemia comprising administering to a patient in need thereof a provided compound or a pharmaceutically acceptable salt thereof and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound or a pharmaceutically acceptable salt thereof and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound or a pharmaceutically acceptable salt thereof and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound or a pharmaceutically acceptable salt thereof and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic maiginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound or a pharmaceutically acceptable salt thereof and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound or a pharmaceutically acceptable salt thereof and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Chuig-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity or degrading a protein kinase in a biological sample comprising the step of contacting said biological sample with a compound of this invention, or a composition comprising said compound.

According to another embodiment, the invention relates to a method of inhibiting or degrading IRAK-1, IRAK-2, and/or IRAK-4, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition and/or degradation of a protein kinase, or a protein kinase selected from IRAK-1, IRAK-2, and/or IRAK-4, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting one or more of IRAK-1, IRAK-2, and/or IRAK-4, or a mutant thereof, activity in a patient comprising the step of administering to said patient a compound of the present invention, or a composition comprising said compound. In other embodiments, the present invention provides a method for treating a disorder mediated by one or more of IRAK-1, IRAK-2, and/or IRAK-4, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO 2008/118802, US 2010/0197686), navitoclax (and analogs thereof, see U.S. Pat. No. 7,390,799), NH-1 (Shenayng Pharmaceutical University), obatoclax (and analogs thereof, see WO 2004/106328, US 2005/0014802), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, CL-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO 2008/039218, US 2008/0108636 and WO 2011/090760, US 2010/0249092, the entirety of each of which is herein incorporated by reference.

Further examples of SYK inhibitory compounds, and conditions treatable by such compounds in combination with compounds of this invention can be found in WO 2003/063794, US 2004/0029902, WO 2005/007623, US 2005/0075306, and WO 2006/078846, US 2006/0211657, the entirety of each of which is herein incorporated by reference.

Further examples of PI3K inhibitory compounds, and conditions treatable by such compounds in combination with compounds of this invention can be found in WO 2004/019973, US 2004/0106569, WO 2004/089925, US 2004/0242631, U.S. Pat. No. 8,138,347, WO 2002/088112, US 2004/0116421, WO 2007/084786, US 2010/0249126, WO 2007/129161, US 2008/0076768, WO 2006/122806, US 2008/0194579, WO 2005/113554, US 2008/0275067, and WO 2007/044729, US 2010/0087440, the entirety of each of which is herein incorporated by reference.

Further examples of JAK inhibitory compounds, and conditions treatable by such compounds in combination with compounds of this invention can be found in WO 2009/114512, US 2009/0233903, WO 2008/109943, US 2010/0197671, WO 2007/053452, US 2007/0191405, WO 2001/0142246, US 2001/0053782, and WO 2007/070514, US 2007/0135461, the entirety of each of which is herein incorporated by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zamestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™),); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vemalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTDR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO 2011/070024, US 2011/0165156, WO 2011/0107553, US 2012/0329997, WO 2011/131407, US 2013/0005949, WO 2013/087699, US 2014/0336363, WO 2013/119716, WO 2013/132044, US 2014/0079706) or FPA-008 (WO 2011/140249, US 2011/0274683; WO 2013/169264; WO 2014/036357, US 2014/0079699).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO 2010/077634, US 2010/0203056), durvalumab (MED14736), BMS-936559 (WO 2007/005874, US 2009/0055944), and MSB0010718C (WO 2013/079174, US 2014/0341917).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO 2010/019570, US 2010/0150892, WO 2014/008218, US 2014/0093511), or IMP-731 or IMP-321 (WO 2008/132601, US 2010/0233183, WO 2009/044273, US 2011/0008331).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO 2006/105021, US 2007/0098719, WO 2009/009116, US 2009/0136494), or MK-4166 (WO 2011/028683, US 2012/0189639).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO 2009/073620, US 2011/053941, WO 2009/132238, US 2011/136796, WO 2011/056652, US 2012/277217, WO 2012/142237, US 2014/066625).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO 2006/029879, U.S. Pat. No. 7,501,496).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO 2011/109400, US 2013/0149236).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, the entirety of each of which is herein incorporated by reference, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams ET. AL. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S. A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron −4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations were performed under reduced pressure, preferably between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention were either commercially available or can be produced by organic synthesis methods known to one of ordinary skill in the art (Houben-Weyl 4th Ed. 1952, Methods of Oiganic Synthesis, Thieme, Volume 21). Further, the compounds of the present invention can be produced by organic synthesis methods known to one of ordinary skill in the art as shown in the following examples.

All reactions were carried out under nitrogen or argon unless otherwise stated.

Proton NMR (¹H NMR) was conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent |
| | Technologies 6110 |
| | Agilent Technologies 1200 series MS: LC/MSD VL |
| NMR | BRUKER AVANCE III/400; Frequency (MHz) 400.13; |
| | Nucleus: 1H; Number of Transients: 8 |
| Prep-HPLC | Gilson GX-281 systems: instruments GX-A, GX-B, |
| | GX-C, GX-D, GX-E, GX-F, GX-G and GX-H |
| GCMS | SHIMADZU GCMS-QP2010 Ultra |
| Analytical | Agilent Technologies 1290 Infinity |
| cSFC | |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase are used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃·H₂O in water (solvent A) and acetonitrile (solvent B).

HPLC Analytical Method: HPLC was carried out on X Bridge C18 150*4.6 mm, 5 micron. Column flow is 1.0 ml/min and mobile phase are used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19)mm, 5μ. Column flow was 16.0 ml/min. Mobile phase used was (A) 0.1% Formic Acid in Water and (B) Acetonitrile. Basic method used was (A) 5 mM ammonium bicarbonate and 0.10% NH₃ in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates:

### N-[2-(4-formylcyclohexyl)-6-(1-hydroxy-1-methyl-ethyl)indazol-5-yl]cyclopropanecarboxamide (Intermediate BOX)

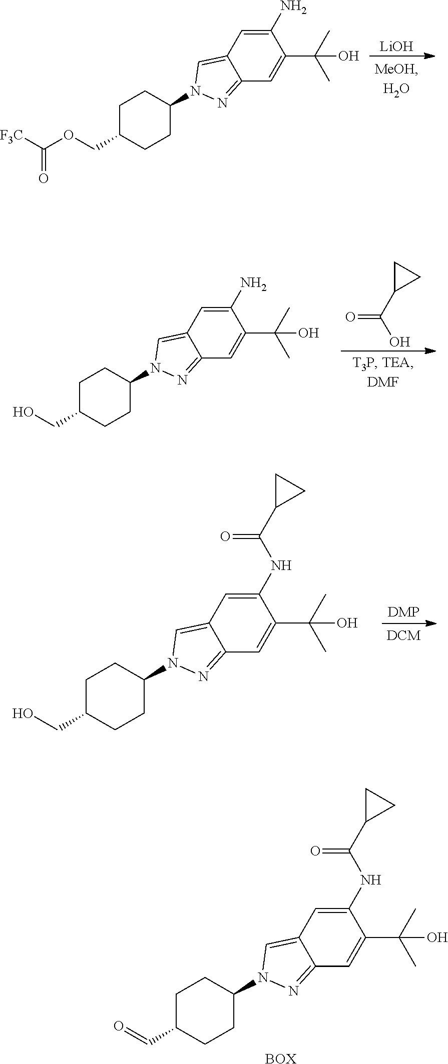

### Step 1—2-[5-Amino-2-[4-(hydroxymethyl)cyclohexyl]indazol-6-yl]propan-2-ol

To a solution of [4-[5-amino-6-(1-hydroxy-1-methyl-ethyl)indazol-2-yl]cyclohexyl]methyl 2,2,2-trifluoroacetate (2.60 g, 6.51 mmol, synthesized via Steps 1-6 of Intermediate AOX) in a mixed solvent of THF (15 mL) and H₂O (5 mL) was added LiOH·H₂O (1.37 g, 32.5 mmol). The reaction mixture was stirred at 60° C. for 20 hrs. On completion, the residue was poured into water (5 mL) and stirred for 5 min. The aqueous phase was then extracted with ethyl acetate (50 mL×2), and the combined organic phase was washed with brine (50 mL×2), dried with anhydrous Na₂SO₄, filtered and concentrated in vacuo. The residue was purified by silica gel chromatography (DCM/MeOH=25/1) to give the title compound (840 mg, 35% yield) as yellow solid. ¹H NMR (400 MHz, DMSO-d₆) δ 7.88 (s, 1H), 7.31 (s, 1H), 6.63 (s, 1H), 5.35-5.15 (m, 3H), 4.48 (t, J=5.2 Hz, 1H), 4.32-4.22 (m, 1H), 3.27 (t, J=5.6 Hz, 2H), 2.12-2.03 (m, 2H), 1.92-1.80 (m, 4H), 1.59 (s, 6H), 1.50-1.40 (m, 1H), 1.18-1.05 (m, 2H); LC-MS (ESI⁺) m/z 304.1 (M+H)⁺.

### Step 2—N-[2-[4-(hydroxymethyl)cyclohexyl]-6-(1-hydroxy-1-methyl-ethyl)indazol-5-yl]cyclopropanecarboxamide

To a solution 2-[5-amino-2-[4-(hydroxymethyl)cyclohexyl]indazol-6-yl]propan-2-ol(100 mg, 274 umol) and cyclopropanecarboxylic acid (23.6 mg, 275 umol, 21.7 uL, CAS #1029691-16-4) in DMF (2 mL) was added T₃P (349 mg, 549 umol, 326 μL, 50% solution), and DIEA (177 mg, 1.37 mmol, 239 uL). The reaction mixture was then stirred at 25° C. for 2 hrs. On completion, the reaction mixture was concentrated in vacuo and the crude product was purified by reversed phase flash (0.1% NH₃·H₂O) to give the title compound (80.0 mg, 73% yield) as white solid. ¹H NMR (400 MHz, DMSO-d₆) δ 10.69 (s, 1H), 8.38-8.22 (m, 2H), 7.51 (s, 1H), 6.18 (s, 1H), 4.48 (t, J=5.2 Hz, 1H), 4.43-4.32 (m, 1H), 3.28-3.26 (m, 2H), 2.15-2.07 (m, 2H), 1.93-1.84 (m, 5H), 1.61 (s, 6H), 1.51-1.43 (m, 1H), 1.14 (dd, J=2.8, 12.4 Hz, 2H), 0.99 (s, 2H), 0.84-0.82 (m, 2H); LC-MS (ESI⁺) m/z 372.2 (M+H)⁺.

### Step 3—N-[2-(4-formylcyclohexyl)-6-(1-hydroxy-1-methyl-ethyl)indazol-5-yl]cyclopropanecarboxamide

To a solution of N-[2-[4-(hydroxymethyl)cyclohexyl]-(1-hydroxy-1-methyl-ethyl)indazol-5-yl]yclopropanecarboxamide (70.0 mg, 176 umol) in DCM (2 mL) was added DMP (372 mg, 878 umol, 272 uL) and the reaction mixture was stirred at 25° C. for 2 hrs. On completion, the mixture was quenched with sat. Na₂S₂O₃ (5 mL) and sat. NaHCO₃ (5 mL). The mixture was then extracted with DCM (2×20 mL). The organic layer was washed with brine (2×10 mL), dried with Na₂SO₄, filtered and the filtrate was concentrated in vacuo to give the title compound (40.0 mg, 29% yield) as white solid. LC-MS (ESI⁺) m/z 370.2 (M+H)⁺.

### [4-(6-Bromo-5-methoxy-1-methyl-benzimidazol-2-yl)cyclohexyl]methanol (Intermediate BOY)

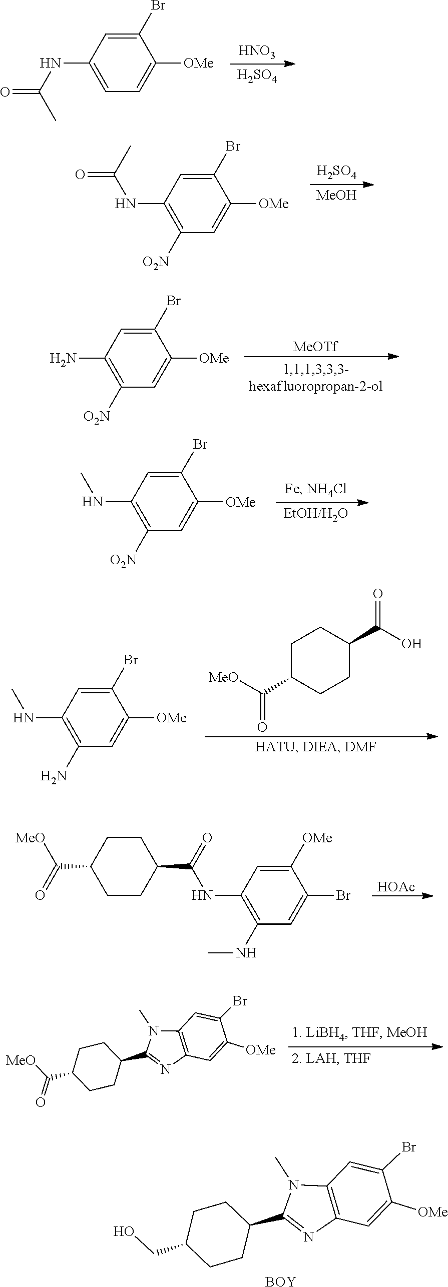

### Step 1—N-(5-bromo-4-methoxy-2-nitro-phenyl)acetamide

HNO₃ (19.6 g, 311 mmol) was added to Ac₂O (38.2 g, 374 mmol), and the mixture was cooled to −10° C., then it was added to a solution of N-(3-bromo-4-methoxy-phenyl)acetamide (7.00 g, 28.7 mmol, CAS #6943-73-3) in Ac₂O (38.2 g, 374 mmol) dropwise at −5° C. The reaction mixture was then stirred at 25° C. for 1 h. On completion, the reaction was poured into cold water (200 mL) and a precipitate formed. The reaction was filtered and the filtered cake was washed with water (20 mL×3). Then the crude filter cake was triturated with EA (40 mL) to afford the title compound (5.50 g, 66% yield) as a yellow solid.

### Step 2—5-Bromo-4-methoxy-2-nitro-aniline

To a solution of N-(5-bromo-4-methoxy-2-nitro-phenyl)acetamide (5.50 g, 19.0 mmol) in MeOH (200 mL) was added H₂SO₄ (18.7 g, 190 mmol) and the mixture was stirred at 70° C. for 2 hours. On completion, the organic solvent was removed under vacuum. Then the reaction was diluted with water (300 mL), extracted with EA (100 mL×3), washed with brine (100 mL), dried over Na₂SO₄, filtered and concentrated under vacuum to afford the title compound (4.00 g, 85% yield) as a yellow solid. LC-MS (ESI⁺) m/z 247.0 (M+H)⁺.

### Step 3—5-Bromo-4-methoxy-N-methyl-2-nitro-aniline

To a solution of 5-bromo-4-methoxy-2-nitro-aniline (4.00 g, 16.2 mmol) in 1,1,1,3,3,3-hexafluoro-propan-2-ol (100 mL) was added methyl trifluoromethanesulfonate (3.99 g, 24.3 mmol) and the mixture was stirred at 25° C. for 3 hours. On completion, the reaction was diluted with EA (100 mL), washed with water (30 mL×3) and brine (4 mL), dried over Na₂SO₄, filtered and concentrated under vacuum to give the crude product. The residue was purified by silica gel column chromatography (PE:EA from 1:0 to 50:1) to afford the title compound (1.80 g, 34% yield) as a red solid. LC-MS (ESI⁺) m/z 261.0 (M+H)⁺.

### Step 4—4-Bromo-5-methoxy-N2-methyl-benzene-1,2-diamine

To a solution of 5-bromo-4-methoxy-N-methyl-2-nitro-aniline (1.70 g, 6.51 mmol) in EtOH (100 mL) and H₂O (30 mL) was added Fe (1.82 g, 32.6 mmol) and NH₄Cl (3.48 g, 65.1 mmol) and the mixture was stirred at 60° C. for 2 hours. On completion, the reaction was filtered through a celite while it was still hot, the filtered cake was washed with EtOH (20 mL×3) and the combined solvent was concentrated in vacuo to afford a crude product. The residue was purified by reversed-phase HPLC (0.1% FA condition) to give the title compound (1.70 g, 90% yield) as an orange solid. LC-MS (ESI⁺) m/z 231.5 (M+H)⁺.

### Step 5—Methyl 4-[[4-bromo-5-methoxy-2-(methylamino)phenyl]carbamoyl]cyclohexanecarboxylate

To a solution of 4-bromo-5-methoxy-N2-methyl-benzene-1,2-diamine (1.70 g, 7.36 mmol) and 4-methoxycarbonylcyclohexanecarboxylic acid (1.51 g, 8.09 mmol, CAS #15177-67-0) in DMF (30 mL) was added DIEA (2.85 g, 22.1 mmol) and HATU (4.20 g, 11.0 mmol) and the mixture was stirred at 25° C. for 1 h. On completion, the reaction was diluted with water (200 mL), and extracted with EA (60 mL×3). The combined organic layer was washed with brine (50 mL), dried over Na₂SO₄, filtered and concentrated under vacuum to afford the title compound (2.20 g, 67% yield) as a yellow oil. LC-MS (ESI⁺) m/z 399.1 (M+H)⁺.

### Step 6—Methyl 4-(6-bromo-5-methoxy-1-methyl-benzimidazol-2-yl)cyclohexanecarboxylate

A solution of methyl 4-[[4-bromo-5-methoxy-2-(methylamino)phenyl]carbamoyl]cyclohexane carboxylate (2.10 g, 5.26 mmol) in HOAc (20 mL) was stirred at 80° C. for 1 hour. On completion, the reaction was concentrated in vacuo to give a crude which was purified by reversed phase HPLC (FA=0.1%) to afford the title compound (1.20 g, 2.83 mmol, 54% yield) as a yellow solid. LC-MS (ESI⁺) m/z 381.1 (M+H)⁺.

### Step 7—[4-(6-Bromo-5-methoxy-1-methyl-benzimidazol-2-yl)cyclohexyl]methanol

To a solution of methyl 4-(6-bromo-5-methoxy-1-methyl-benzimidazol-2-yl)cyclohexanecarboxylate (1.00 g, 2.62 mmol) in THF (16 mL) and MeOH (2 mL) was added LiBH₄ (286 mg, 13.1 mmol) at 0° C. Then the mixture was stirred at 60° C. for 16 hours. Next, LiBH₄ (457 mg, 21.0 mmol) was added and the mixture was stirred at 60° C. for another 16 h. On completion, the reaction was poured into water (100 mL), extracted with EA (3×30 mL), washed with brine (30 mL), dried over Na₂SO₄, filtered and concentrated under vacuum to give the crude product. The residue was purified by silica gel column chromatography (PE:EA from 10:1 to 0:1) to give the title compound (140 mg, 357 umol, 14% yield) as a brown oil and remaining starting material (300 mg, 708 umol, 27% yield) as a yellow solid. LC-MS (ESI⁺) m/z 353.1 (M+H)⁺.

Next, to a solution of methyl 4-(6-bromo-5-methoxy-1-methyl-benzimidazol-2-yl)cyclohexanecarboxylate (250 mg, 656 umol) in THF (8 mL) was added LAH (40.0 mg, 1.05 mmol) at 0° C., and the mixture was stirred at 0° C. for 0.5 h. On completion, the reaction was quenched with water (2.4 mL), filtered and the filtered cake was washed with MeOH (5 mL×3), then the solvent was removed under vacuum. Then the crude was purified by prep-TLC to afford the title compound (140 mg, 357 umol, 54.4% yield) as brown oil. LC-MS (ESI⁺) m/z 353.1 (M+H)⁺.

### N-[2-(4-formylcyclohexyl)-6-methoxy-3-methyl-benzimidazol-5-yl]-6-(trifluoromethyl) pyridine-2-carboxamide (Intermediate BOZ)

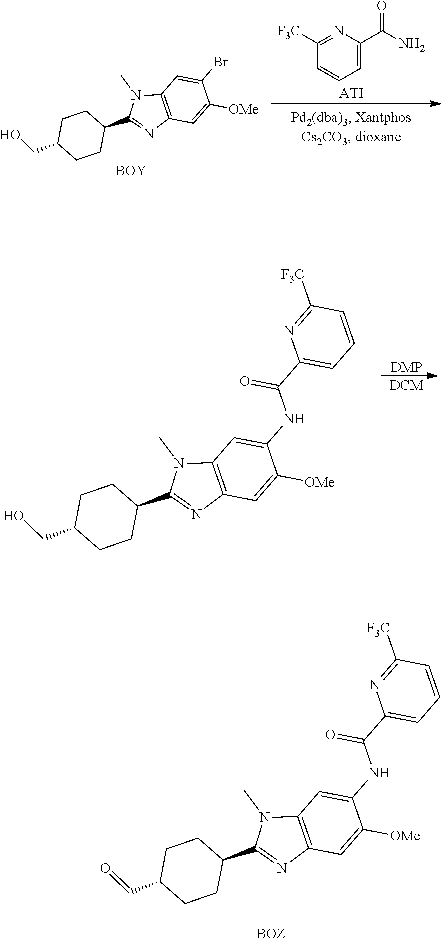

### Step 1—N-[2-[4-(hydroxymethyl)cyclohexyl]-6-methoxy-3-methyl-benzimidazol-5-yl]-6-(trifluoro-methyl)pyridine-2-carboxamide

To a solution of [4-(6-bromo-5-methoxy-1-methyl-benzimidazol-2-yl)cyclohexyl]methanol (250 mg, 708 umol, Intermediate BOY) and 6-(trifluoromethyl)pyridine-2-carboxamide (269 mg, 1.42 mmol, Intermediate ATI) in dioxane (4 mL) was added Cs₂CO₃ (461 mg, 1.42 mmol), Xantphos (81.9 mg, 141 umol) and Pd₂(dba)₃ (64.8 mg, 70.8 umol). The mixture was degassed and purged with N₂ three times and then it was stirred at 100° C. for 16 hours. On completion, the reaction was concentrated under vacuum to give a crude which was purified by reversed-phase HPLC (0.1% NH3·H₂O condition) to afford the title compound (90.0 mg, 175 umol, 25% yield) as a yellow solid. LC-MS (ESI⁺) m/z 463.3 (M+H)⁺.

### Step 2—N-[2-(4-formylcyclohexyl)-6-methoxy-3-methyl-benzimidazol-5-yl]-6-(trifluoromethyl) pyridine-2-carboxamide

To a solution of N-[2-[4-(hydroxymethyl)cyclohexyl]-6-methoxy-3-methyl-benzimidazol-5-yl]-6-(trifluoromethyl)pyridine-2-carboxamide (90.0 mg, 195 umol) in DCM (4 mL) was added DMP (165 mg, 389 umol) and the mixture was stirred at 25° C. for 1 hour. On completion, the reaction was quenched with sat. Na₂S₂CO₃ (10 mL), then diluted with EA (100 mL), washed with sat. NaHCO₃ (100 mL) and brine (40 mL). The mixture was then dried over Na₂SO₄, filtered and concentrated under vacuum to give the title compound (80.0 mg, 85% yield) as a yellow solid. LC-MS (ESI⁺) m/z 461.2 (M+H)⁺.

### 2-(2,6-Dioxo-3-piperidyl)-4-[2-(4-piperidyl)ethylamino]isoindoline-1,3-dione (Intermediate BPA)

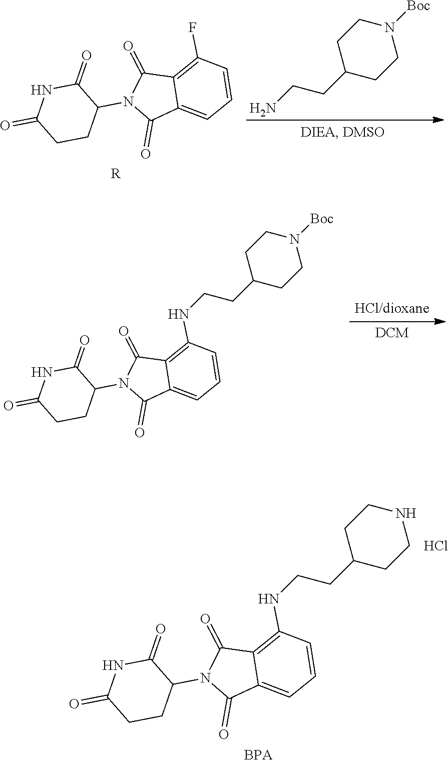

### Step 1—Tert-butyl 4-[2-[[2-(2,6-dioxo-3-piperidyl)-1,3-dioxo-isoindolin-4-yl]amino]ethyl]piperidine-1-carboxylate

To a mixture of 2-(2,6-dioxo-3-piperidyl)-4-fluoro-isoindoline-1,3-dione (2.10 g, 7.60 mmol, Intermediate R) and tert-butyl 4-(2-aminoethyl)piperidine-1-carboxylate (1.74 g, 7.60 mmol, CAS #146093-46-1) in DMSO (60 mL) was added DIEA (1.96 g, 15.2 mmol). The mixture was stirred at 130° C. for 4 hours. On completion, the mixture was purified by reverse phase flash (FA condition) to give the title compound (2.00 g, 51% yield). ¹H NMR (400 MHz, DMSO-d₆) δ 11.09 (s, 1H), 7.58 (dd, J=7.2, 8.3 Hz, 1H), 7.09 (d, J=8.4 Hz, 1H), 7.02 (d, J=7.2 Hz, 1H), 6.51 (t, J=6.0 Hz, 1H), 5.05 (dd, J=5.6, 12.8 Hz, 1H), 3.96-3.85 (m, 2H), 2.94-2.81 (m, 1H), 2.75-2.65 (m, 2H), 2.60 (m, 1H), 2.56-2.54 (m, 1H), 2.08 -1.98 (m, 1H), 1.69 (d, J=12.4 Hz, 2H), 1.52 (m, 3H), 1.38 (s, 11H), 1.04-1.01 (m, 2H); LC-MS (ESI⁺) m/z 385.2 (M+H)⁺.

### Step 2—2-(2,6-Dioxo-3-piperidyl)-4-[2-(4-piperidyl)ethylamino]isoindoline-1,3-dione

To a solution of tert-butyl 4-[2-[[2-(2,6-dioxo-3-piperidyl)-1,3-dioxo-isoindolin-4-yl]amino]ethyl]piperidine-1-carboxylate (100 mg, 206 umol) in DCM (3 mL) was added HCl/dioxane (4 M, 1.5 mL) at 25° C. and the mixture was stirred at 25° C. for 1 hour. On completion, the mixture was concentrated in vacuo to give the title compound (135 mg, 98% yield, HCl salt) as light yellow solid. LC-MS (ESI⁺) m/z 385.2 (M+H)⁺.

### N-(2-((1R,4R)-4-formylcyclohexyl)-7-methoxyimidazo[1,2-a]pyridin-6-yl)-6-(trifluoro methyl)picolinamide (Intermediate BPB)

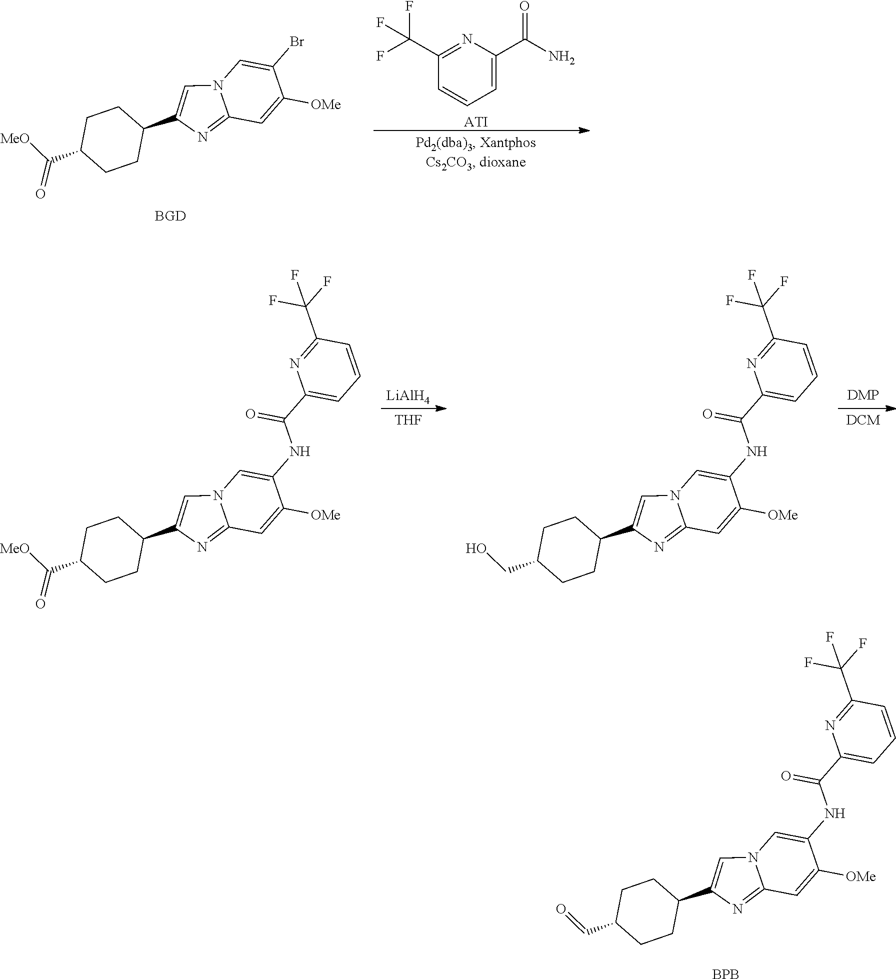

### Step 1—(1R,4R)-methyl 4-(7-methoxy-6-(6-(trifluoromethyl)picolinamido)imidazo[1,2-a]pyridin-2-yl) cyclohexanecarboxylate

To a solution of methyl 4-(6-bromo-7-methoxy-imidazo[1,2-a]pyridin-2-yl)cyclohexanecarboxylate (400 mg, 1.09 mmol, Intermediate BGD) and 6-(trifluoromethyl)pyridine-2-carboxamide (227 mg, 1.20 mmol, Intermediate ATI) in dioxane (5 mL) was added Pd₂(dba)₃ (99.7 mg, 108 umol), Cs₂CO₃ (709 mg, 2.18 mmol) and Xantphos (126 mg, 217 umol). The mixture was stirred at 80° C. for 12 hours under N₂ atmosphere. On completion, the reaction mixture was filtered and concentrated under reduced pressure to give a residue. The residue was purified by column chromatography (SiO₂, DCM/MeOH=100/1 to 20/1) to give the title compound (280 mg, 40% yield) as an off-white solid. LC-MS (ESI⁺) m/z 477.4 (M+H)⁺.

### Step 2—N-(2-((1R,4R)-4-(hydroxymethyl)cyclohexyl)-7-methoxyimidazo[1,2-a]pyridine-6-yl)-6-(trifluoromethyl)picolinamide

To a solution of methyl 4-[7-methoxy-6-[[6-(trifluoromethyl)pyridine-2-carbonyl]amino]imidazo[1, 2-a]pyridin-2-yl]cyclohexanecarboxylate (180 mg, 377 umol) in THF (5 mL) was added LiAlH₄ (28.6 mg, 755 umol) at 0° C., and the mixture was stirred at 0° C. for 0.5 hour. On completion, the reaction mixture was quenched with H₂O (0.1 mL), 15% NaOH aqueous (0.2 mL) and H₂O (0.1 mL) at 0° C. Then the mixture was diluted with THF (50 mL) and dried over Na₂SO₄, filtered and concentrated under reduced pressure to give the title compound (160 mg, 94% yield) as a brown solid. LC-MS (ESI⁺) m/z 449.4 (M+H)⁺.

### Step 3—N-(2-((1R,4R)-4-formylcyclohexyl)-7-methoxyimidazo[1,2-a]pyridin-6-yl)-6-(trifluoro methyl)picolinamide

To a solution of N-[2-[4-(hydroxymethyl)cyclohexyl]-7-methoxy-imidazo[1,2-a]pyridin-6-yl]-6-(trifluoromethyl)pyridine-2-carboxamide (160 mg, 356 umol) in DCM (5 mL) was added DMP (227 mg, 535 umol) and the mixture was stirred at 25° C. for 0.5 hour. On completion, the reaction mixture was quenched with sat. Na₂SO₃ (20 mL) and sat. NaHCO₃ (20 mL) at 0° C., and then diluted with H₂O (20 mL) and extracted with DCM (3×50 mL). The combined organic layers were washed with brine (50 mL), dried over Na₂SO₄, filtered and concentrated under reduced pressure to give a residue. The residue was purified by prep-TLC (SiO₂, DCM:MeOH=20:1) to give the title compound (88.0 mg, 48% yield) as an off-white solid. LC-MS (ESI⁺) m/z 447.1 (M+H)⁺.

### N-(2-((1r,4r)-4-formylcyclohexyl)-7-(2-hydroxypropan-2-yl)imidazo[1,2-a]pyridin-6-yl)-6-(trifluoromethyl)picolinamide (Intermediate BPC)

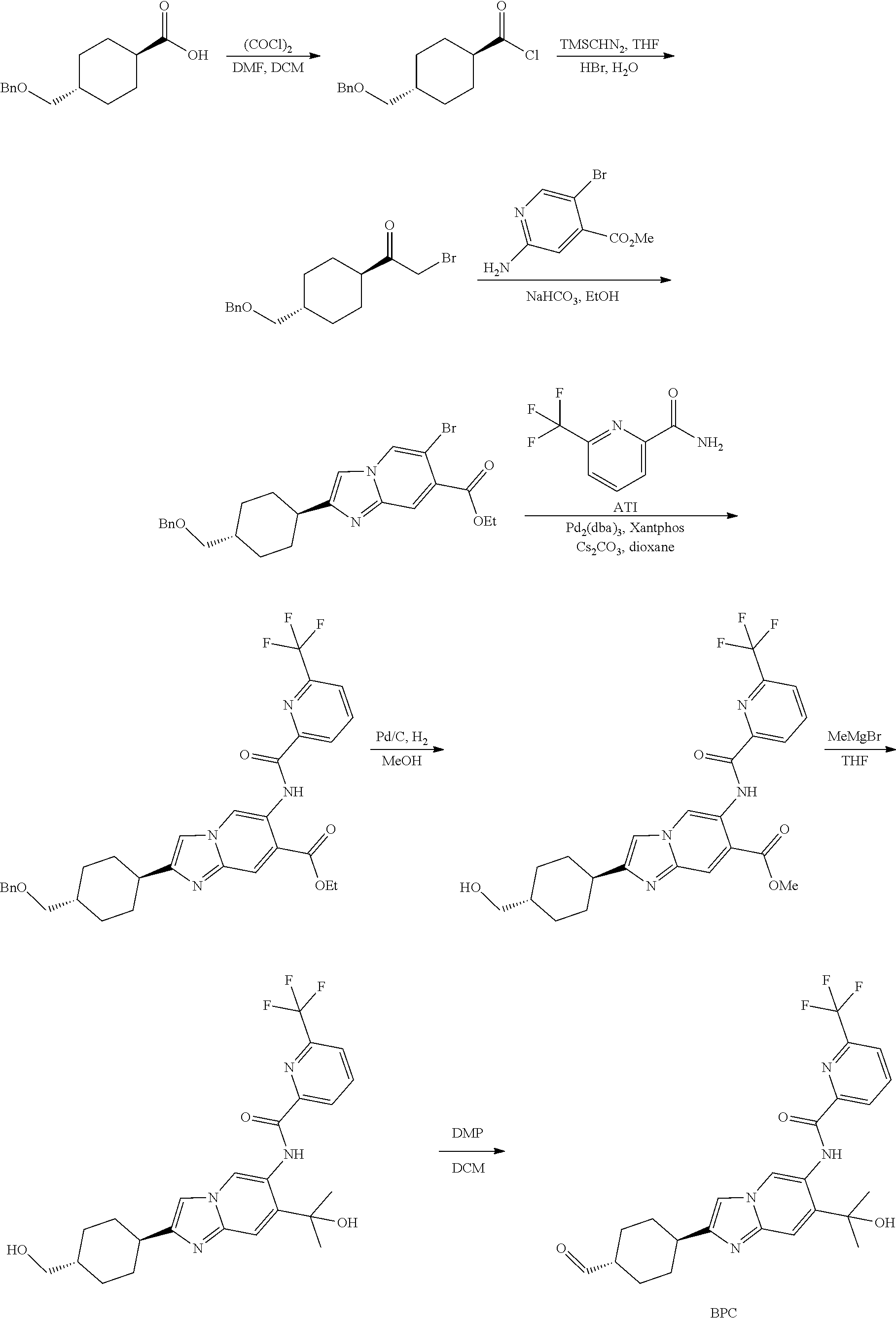

### Step 1—(1R, 4R)-4-((benzyloxy)methyl)cyclohexanecarbonyl chloride

To a solution of 4-(benzyloxymethyl)cyclohexanecarboxylic acid (4.00 g, 16.1 mmol, synthesized via Steps 1-3 of Intermediate BAU) in DCM (20 mL) was added DMF (117 mg, 1.61 mmol) and dropwise (COCl)₂ (4.50 g, 35.4 mmol) at 0° C. Then the mixture was stirred at 25° C. for 1 hour. On completion, the reaction mixture was concentrated in vacuo to give the title compound (4.00 g, 93% yield) as brown oil.

### Step 2—1-((1R, 4R)-4-((benzyloxy)methyl)cyclohexyl)-2-bromoethanone

A solution of 4-(benzyloxymethyl)cyclohexanecarbonyl chloride (4.00 g, 14.9 mmol) in THF (20 mL) was added dropwise to TMSCHN₂ (2 M, 14.9 mL, 29.9 mmol) at 0° C.. Then the reaction mixture was stirred at 0° C. for 1 hour and at 25° C. for 1 hour. Next, HBr (5.56 g, 32.9 mmol, 48% solution) was added dropwise and the mixture was stirred at 0° C. for 0.5 hour. On completion, the reaction mixture was quenched with sat. NaHCO₃ (100 mL) at 0° C., and then diluted with H₂O (50 mL) and extracted with EA (3×50 mL). The combined organic layers were washed with brine (50 mL), dried over Na₂SO₄, filtered and concentrated in vacuo to give a residue. The residue was purified by column chromatography (SiO₂, petroleum ether/ethyl acetate=100/1 to 3/1) to give the title compound (1.30 g, 24% yield) as an off-white solid. ¹H NMR (400 MHz, chloroform-d) δ 7.39-7.28 (m, 5H), 4.50 (s, 2H), 4.17 (s, 1H), 3.97 (s, 1H), 3.67 (s, 1H), 3.31 (d, J=6.1 Hz, 2H), 2.77-2.57 (m, 1H), 2.04-1.84 (m, 4H), 1.73-1.55 (m, 1H), 1.50-1.33 (m, 2H), 1.17-0.95 (m, 2H); LC-MS (ESI⁺) m/z 325.2 (M+H)⁺.

### Step 3—Ethyl 2-((1r, 4r)-4-((benzyloxy)methyl)cyclohexyl)-6-bromoimidazo[1,2-a]pyridine-7-carboxylate

To a solution of 1-[4-(benzyloxymethyl)cyclohexyl]-2-bromo-ethanone (1.30 g, 4.00 mmol) and methyl 2-amino-5-bromo-pyridine-4-carboxylate (923 mg, 4.00 mmol, CAS #882499-87-8) in EtOH (20 mL) was added NaHCO₃ (671 mg, 7.99 mmol) and the mixture was stirred at 90° C. for 12 hours. On completion, the reaction mixture was concentrated under reduced pressure to remove the solvent. The residue was diluted with H₂O (100 mL) and extracted with DCM (3×50 mL). The combined organic layers were washed with brine (50 mL), dried over Na₂SO₄, filtered and concentrated under reduced pressure to give a residue. The residue was purified by column chromatography (SiO₂, DCM/MeOH=100/1 to 20/1) to give the title compound (0.45 g, 20% yield) as brown oil. ¹H NMR (400 MHz, chloroform-d) δ 8.31 (s, 1H), 8.19 (s, 1H), 7.39 (s, 1H), 7.37-7.32 (m, 5H), 4.53 (s, 2H), 4.51-4.49 (m, 2H), 4.41 (m, 2H), 3.35 (m, 2H), 3.31-3.26 (m, 2H), 2.77 (s, 1H), 2.23-2.15 (m, 2H), 2.00 (m, 2H), 1.95-1.82 (m, 3H), 1.79-1.67 (m, 2H), 1.53 (m, 2H), 1.41 (m, 4H), 1.25 (m, 3H), 1.18 (m, 2H), 1.07-0.95 (m, 2H); LC-MS (ESI⁺) m/z 471.3 (M+H)⁺.

### Step 4—Ethyl 2-((1r,4r)-4-((benzyloxy)methyl)cyclohexyl)-6-(6-(trifluoromethyl)picolinamido) imidazo[1,2-a]pyridine-7-carboxylate

To a solution of ethyl 2-[4-(benzyloxymethyl)cyclohexyl]-6-bromo-imidazo[1,2-a]pyridine-7-carboxylate (400 mg, 848 umol) and 6-(trifluoromethyl)pyridine-2-carboxamide (177 mg, 933 umol, Intermediate ATI) in dioxane (4 mL) was added Pd₂(dba)₃ (77.7 mg, 84.8 umol), Cs₂CO₃ (552 mg, 1.70 mmol) and Xantphos (98.2 mg, 169 umol). The mixture was then stirred at 80° C. for 12 hours under nitrogen atmosphere. On completion, the reaction mixture was filtered and concentrated under reduced pressure to give a residue. The residue was purified by column chromatography (SiO₂, DCM/MeOH=100/1 to 20/1) to give the title compound (0.30 g, 29% yield) as brown oil. LC-MS (ESI⁺) m/z 581.5 (M+H)⁺.

### Step 5—Ethyl 2-((1r,4r)-4-(hydroxymethyl)cyclohexyl)-6-(6-(trifluoromethyl)picolinamido)imidazo [1,2-a]pyridine-7-carboxylate

To a solution of ethyl 2-[4-(benzyloxymethyl)cyclohexyl]-6-[[6-(trifluoromethyl)pyridine-2-carbonyl]amino]imidazo[1,2-a]pyridine-7-carboxylate (300 mg, 516 umol) in MeOH (3 mL) and THF (0.5 mL) was added Pd/C (10 wt %, 30.0 mg) under N₂ atmosphere. The suspension was degassed and purged with H₂ three times. The mixture was stirred under H₂ (50 Psi.) at 25° C. for 2 hours. On completion, the reaction mixture was filtered and concentrated under reduced pressure to give the title compound (90.0 mg, 36% yield) as brown oil. LC-MS (ESI⁺) m/z 477.4 (M+H)⁺.

### Step 6—N-(2-((1r,4r)-4-(hydroxymethyl)cyclohexyl)-7-(2-hydroxypropan-2-yl)imidazo[1,2-a]pyridin-6-yl)-6-(trifluoromethyl)picolinamide

To a solution of methyl 2-[4-(hydroxymethyl)cyclohexyl]-6-[[6-(trifluoromethyl)pyridine-2-carbonyl]amino]imidazo[1,2-a]pyridine-7-carboxylate (90.0 mg, 188 umol) in THF (1 mL) was added MeMgBr (3.0 M, 629 uL) dropwise at 0° C. The mixture was stirred at 0° C. for 1 hour. On completion, the reaction mixture was quenched with sat. NH₄Cl (20 mL) at 0° C., and then diluted with H₂O (20 mL) and extracted with DCM (3×50 mL). The combined organic layers were washed with brine (50 mL), dried over Na₂SO₄, filtered and concentrated under reduced pressure to give the title compound (90.0 mg, 100% yield) as brown oil. LC-MS (ESI⁺) m/z 477.4 (M+H)⁺.

### Step 7—N-(2-((1r,4r)-4-formylcyclohexyl)-7-(2-hydroxypropan-2-yl)imidazo[1,2-a]pyridin-6-yl)-6-(trifluoromethyl)picolinamide

To a solution of N-[2-[4-(hydroxymethyl)cyclohexyl]-7-(1-hydroxy-1-methyl-ethyl) imidazo[1,2-a]pyridin-6-yl]-6-(trifluoromethyl)pyridine-2-carboxamide (90.0 mg, 188 umol) in DCM (2 mL) was added DMP (104 mg, 245 umol) at 0° C. Then the mixture was stirred at 25° C. for 0.5 hour. On completion, the reaction mixture was quenched with sat. NaHCO₃ (20 mL) and sat. Na₂SO₃ (20 mL) at 0° C., and then diluted with H₂O (20 mL) and extracted with DCM (3×50 mL). The combined organic layers were washed with brine (50 mL), dried over Na₂SO₄, filtered and concentrated under reduced pressure to give a residue. The residue was purified by prep-TLC (SiO₂, DCM:MeOH=20:1) to give the title compound (58.0 mg, 58% yield) as an off-white solid. ¹H NMR (400 MHz, chloroform-d) δ 12.16 (s, 1H), 9.69 (d, J=1.1 Hz, 1H), 9.57 (s, 1H), 8.47 (d, J=7.8 Hz, 1H), 8.14 (t, J=7.8 Hz, 1H), 7.87 (d, J=7.7 Hz, 1H), 7.53 (s, 1H), 7.34 (s, 1H), 2.74 (m, 1H), 2.48 (m, 1H), 2.42-2.33 (m, 1H), 2.38-2.31 (m, 2H), 2.26 (m, 2H), 1.77 (s, 6H), 1.61 (m, 2H), 1.44 (m, 2H); LC-MS (ESI⁺) m/z 475.4 (M+H)⁺.

### N-[2-(4-formylcyclohexyl)-6-(1-hydroxy-1-methyl-ethyl)-3H-benzimidazol-5-yl]-6-(trifluoromethyl)pyridine-2-carboxamide (Intermediate BPD)

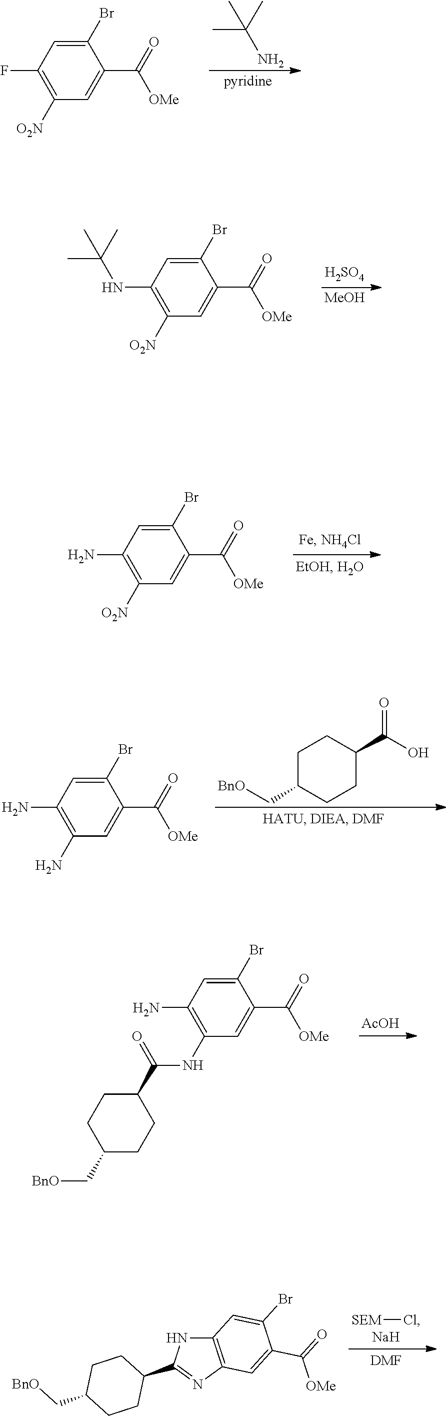

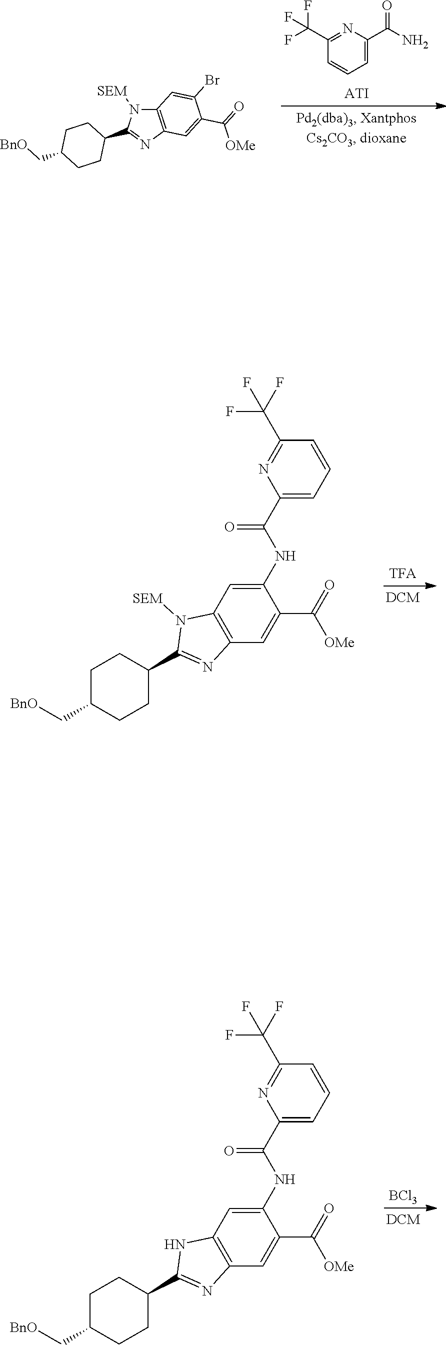

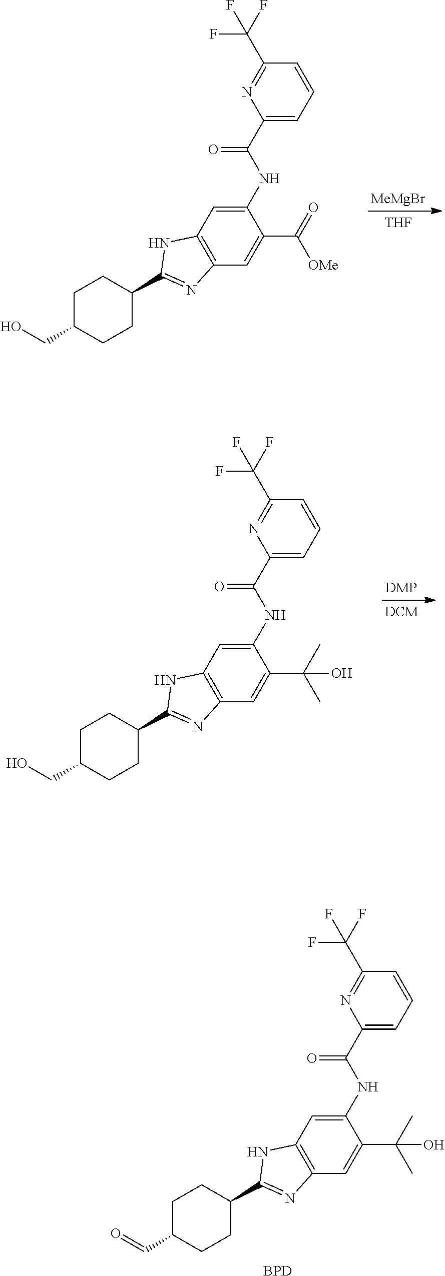

### Step 1—Methyl 2-bromo-4-(tert-butylamino)-5-nitro-benzoate

To a solution of methyl 2-bromo-4-fluoro-5-nitro-benzoate (11.0 g, 39.5 mmol, synthesized via Steps 1-2 of Intermediate BEA) in pyridine (80 mL) was added 2-methylpropan-2-amine (5.79 g, 79.1 mmol, CAS #75-64-9) and the reaction mixture was stirred at 20° C. for 3 hrs. On completion, the reaction mixture was diluted with water (100 mL) and extracted with EA (3×90 mL). The combined organic layers were washed with water (3×80 mL), 1.0 M aq. HCl (2×60 mL), and brine (2×60 mL), then dried over Na₂SO₄, filtered and concentrated in vacuo. The residue was purified by silica gel chromatography (PE:EA=100:1) to give the title compound (9.60 g, 73% yield) as yellow solid. ¹H NMR (400 MHz, CDCl₃) δ 8.85 (s, 1H), 8.53 (s, 1H), 7.40 (s, 1H), 3.90 (s, 3H), 1.54 (s, 9H).

### Step 2—Methyl 4-amino-2-bromo-5-nitro-benzoate

To a solution of methyl 2-bromo-4-(tert-butylamino)-5-nitro-benzoate (3.50 g, 10.5 mmol) in MeOH (30 mL) was added H₂SO₄ (2.76 g, 28.1 mmol, 1.5 mL) at 20° C. The reaction mixture was then stirred at 80° C. for 16 hrs. On completion, the reaction mixture was concentrated in vacuo. The residue was diluted with water (30 mL), basified with saturated NaHCO₃ until the pH=8 and extracted with EA (3×120 mL). The combined organic layers were washed with brine (2×60 mL) dried over Na₂SO₄, filtered and concentrated in vacuo to give the title compound (2.70 g, 92% yield) as yellow solid. ¹H NMR (400 MHz, DMSO-d₆) δ 8.53 (s, 1H), 7.96 (s, 2H), 7.38 (s, 1H), 3.80 (s, 3H).

### Step 3—Methyl 4,5-diamino-2-bromo-benzoate

To a solution of methyl 4-amino-2-bromo-5-nitro-benzoate (2.50 g, 9.09 mmol) and NH₄Cl (3.89 g, 72.7 mmol) in a mixed solvents of EtOH (20 mL) and water (20 mL) was added Fe (2.54 g, 45.4 mmol) at 80° C. and the reaction mixture was stirred at 80° C. for 2 hrs. On completion, the reaction mixture was filtered and the filter cake was washed with EtOH (50 mL). The filtrate was concentrated in vacuo to give a residue which was diluted with water (80 mL) and extracted with DCM (3×100 mL). The combined organic layers were washed with brine, dried over Na₂SO₄, filtered and concentrated in vacuo. The residue was purified by silica gel chromatography (PE:EA=3:1) to give the title compound (1.90 g, 85% yield) as brown solid. ¹H NMR (400 MHz, CDCl₃) δ 7.33 (s, 1H), 6.94 (s, 1H), 3.87 (s, 3H), 3.80 (s, 2H), 3.33 (s, 2H).

### Step 4—Methyl 4-amino-5-[[4-(benzyloxymethyl)cyclohexanecarbonyl]amino]-2-bromo-benzoate

To a solution of methyl 4,5-diamino-2-bromo-benzoate (1.80 g, 7.34 mmol), 4-(benzyloxymethyl)cyclohexanecarboxylic acid (1.82 g, 7.34 mmol, synthesized via Steps 1-3 of Intermediate BAU) and DIEA (1.90 g, 14.6 mmol, 2.56 mL) in DMF (25 mL) was added HATU (3.35 g, 8.81 mmol) and the reaction mixture was stirred at 20° C. for 2 hrs. On completion, the reaction mixture was poured into water (150 mL) and extracted with EA (3×100 mL). The combined organic layers were washed with brine (2×50 mL), dried over Na₂SO₄, filtered and concentrated in vacuo. The residue was purified by silica gel chromatography (PE:EA=4:1) to give the title compound (2.70 g, 77% yield) as brown solid. ¹H NMR (400 MHz, CDCl₃) δ 7.94 (s, 1H), 7.63 (s, 1H), 7.28-7.20 (m, 5H), 6.94 (s, 1H), 4.43 (s, 2H), 4.26 (s, 2H), 3.77 (s, 3H), 3.24 (d, J=6.0 Hz, 2H), 2.24-2.12 (m, 1H), 1.99-1.85 (m, 4H), 1.65-1.59 (m, 1H), 1.56-1.45 (m, 2H), 1.08-0.90 (m, 2H).

### Step 5—Methyl 2-[4-(benzyloxymethyl)cyclohexyl]-6-bromo-1H-benzimidazole-5-carboxylate

A solution of methyl 4-amino-5-[[4-(benzyloxymethyl)cyclohexanecarbonyl]amino]-2-bromo-benzoate (2.50 g, 5.26 mmol) in AcOH (25 mL) was stirred at 80° C. for 12 hrs. On completion, the reaction mixture was concentrated in vacuo to remove AcOH. The residue was diluted with DCM (150 mL), washed with saturated NaHCO₃ (2×40 mL), brine (2×40 mL), dried over Na₂SO₄, filtered and concentrated in vacuo. The residue was purified by silica gel chromatography (PE:EA=4:1) to give the title compound (2.00 g, 83% yield) as brown oil. ¹H NMR (400 MHz, DMSO-d₆) δ 8.01-7.64 (m, 2H), 7.40-7.29 (m, 5H), 4.47 (s, 2H), 3.85 (s, 3H), 3.32-3.30 (m, 2H), 2.88-2.76 (m, 1H), 2.14-2.05 (m, 2H), 1.93-1.85 (m, 2H), 1.71-1.53 (m, 3H), 1.16-1.08 (m, 2H).

### Step 6—Methyl 2-[4-(benzyloxymethyl)cyclohexyl]-6-bromo-1-(2-trimethylsilylethoxymethyl) benzimidazole-5-carboxylate

To a solution of methyl 2-[4-(benzyloxymethyl)cyclohexyl]-6-bromo-1H-benzimidazole-5-carboxylate (1.80 g, 3.94 mmol) in DMF (20 mL) was added NaH (204 mg, 5.12 mmol, 60% dispersion in mineral oil) at 0° C. After 10 min, SEM-Cl (787 mg, 4.72 mmol,) was added and the reaction mixture was stirred at 0° C. for 50 min. On completion, the reaction mixture was poured into saturated NH₄Cl (100 mL) and extracted with EA (3×80 mL). The combined organic layers were washed with brine (60 mL), dried over Na₂SO₄, filtered and concentrated in vacuo. The residue was purified by silica gel chromatography (PE:EA=10:1) to give the title compound (1.53 g, 66% yield) as yellow oil. ¹H NMR (400 MHz, CDCl₃) δ 8.26-7.67 (m, 2H), 7.37-7.30 (m, 5H), 5.48 (d, J=16.0 Hz, 2H), 4.54 (s, 2H), 3.95 (d, J=5.2 Hz, 3H), 3.55 (t, J=8.0 Hz, 2H), 3.37 (d, J=6.0 Hz, 2H), 2.92-2.85 (m, 1H), 2.08-2.00 (m, 4H), 1.87-1.78 (m, 2H), 1.31-1.12 (m, 3H), 0.92 (t, J=8.0 Hz, 2H), −0.03 (d, J=3.6 Hz, 9H).

### Step 7—Methyl 2-[4-(benzyloxymethyl)cyclohexyl]-6-[[6-(trifluoromethyl)pyridine-2-carbonyl]amino]-1-(2-trimethylsilylethoxymethyl)benzimidazole-5-carboxylate

A mixture of methyl 2-[4-(benzyloxymethyl)cyclohexyl]-6-bromo-1-(2-trimethylsilylethoxymethyl) benzimidazole-5-carboxylate (1.60 g, 2.72 mmol), 6-(trifluoromethyl)pyridine-2-carboxamide (517 mg, 2.72 mmol, Intermediate ATI), Pd₂(dba)₃ (249 mg, 272 umol), Xantphos (315 mg, 544 umol) and Cs₂CO₃ (1.77 g, 5.45 mmol) in dioxane (20 mL) was stirred at 80° C. for 4 hrs. On completion, the reaction mixture was filtered and the filter cake was washed with DCM (50 mL). The filtrate was concentrated in vacuo and the residue was purified by silica gel chromatography (PE:EA=8:1 to 4:1) to give the title compound (1.50 g, 79% yield) as yellow oil. ¹H NMR (400 MHz, CDCl3) δ 13.35-12.71 (m, 1H), 9.49-9.03 (m, 1H), 8.57-8.39 (m, 2H), 8.17-8.07 (m, 1H), 7.93-7.83 (m, 1H), 7.38-7.30 (m, 5H), 5.54 (d, J=9.6 Hz, 2H), 4.54 (s, 2H), 4.04 (d, J=8.8 Hz, 3H), 3.65-3.56 (m, 2H), 3.38 (dd, J=1.2, 6.4 Hz, 2H), 3.01-2.87 (m, 1H), 2.14-2.06 (m, 2H), 2.05-1.96 (m, 2H), 1.95-1.82 (m, 3H), 1.24-1.14 (m, 2H), 1.00-0.90 (m, 2H), −0.04 (d, J=5.6 Hz, 9H).

### Step 8—Methyl 2-[4-(benzyloxymethyl)cyclohexyl]-6-[[6-(trifluoromethyl)pyridine-2-carbonyl]amino]-1H-benzimidazole-5-carboxylate

To a solution of methyl 2-[4-(benzyloxymethyl)cyclohexyl]-6-[[6-(trifluoromethyl)pyridine-2-carbonyl]amino]-1-(2-trimethylsilylethoxymethyl)benzimidazole-5-carboxylate (1.00 g, 1.44 mmol) in DCM (10 mL) was added TFA (15.4 g, 135 mmol, 10.0 mL) and the reaction mixture was stirred at 20° C. for 12 hrs. On completion, the combined reaction mixture was concentrated in vacuo. The residue was diluted with DCM (200 mL), washed with sat. NaHCO₃ (2×40 mL), brine (2×40 mL), dried over Na₂SO₄, filtered and concentrated in vacuo. The residue was purified by silica gel chromatography (PE:EA=4:1) to give the title compound (620 mg, 76% yield) as yellow solid. ¹H NMR (400 MHz, CDCl₃) δ 13.48-12.92 (m, 1H), 9.99-9.48 (m, 1H), 9.10 (s, 1H), 8.55-8.45 (m, 2H), 8.11 (t, J=7.6 Hz, 1H), 7.86 (dd, J=0.8, 7.6 Hz, 1H), 7.37-7.30 (m, 5H), 4.51 (s, 2H), 4.03 (s, 3H), 3.32 (d, J=6.4 Hz, 2H), 2.92-2.77 (m, 1H), 2.29-2.15 (m, 2H), 2.00-1.94 (m, 2H), 1.72-1.63 (m, 3H), 1.19-1.03 (m, 2H).

### Step 9—Methyl 2-[4-(hydroxymethyl)cyclohexyl]-6-[[6-(trifluoromethyl)pyridine-2-carbonyl]amino]—1H-benzimidazole-5-carboxylate

To a solution of methyl 2-[4-(benzyloxymethyl)cyclohexyl]-6-[[6-(trifluoromethyl)pyridine-2-carbonyl]amino]-1H-benzimidazole-5-carboxylate (750 mg, 1.32 mmol) in DCM (10 mL) was added a BCl₃-DCM solution (1 M, 13.2 mL) and the reaction mixture was stirred at 20° C. for 1 hr. On completion, the reaction mixture was quenched by saturated NaHCO₃ until the pH=8 and extracted with DCM/MeOH=10:1 (3×80 mL). The combined organic layers were washed with brine (2×50 mL), dried over Na₂SO₄, filtered and concentrated in vacuo. The residue was purified by reverse phase (0.1% FA) to give the title compound (430 mg, 68% yield) as yellow solid, ¹H NMR (400 MHz, DMSO-d₆) δ 13.02-12.76 (m, 1H), 12.57-12.42 (m, 1H), 9.01-8.91 (m, 1H), 8.53-8.45 (m, 1H), 8.39 (t, J=8.0 Hz, 1H), 8.29-8.10 (m, 2H), 3.99-3.89 (m, 3H), 3.28-3.27 (m, 1H), 3.17 (d, J=5.2 Hz, 2H), 2.82 (d, J=12.4 Hz, 1H), 2.15-2.05 (m, 2H), 1.92-1.82 (m, 2H), 1.64-1.53 (m, 2H), 1.49-1.40 (m, 1H), 1.15-1.98 (m, 2H).

### Step 10—N-[2-[4-(hydroxymethyl)cyclohexyl]-6-(1-hydroxy-1-methyl-ethyl)-3H-benzimidazol-5-yl]-6-(trifluoromethyl)pyridine-2-carboxamide

To a solution of methyl 2-[4-(hydroxymethyl)cyclohexyl]-6-[[6-(trifluoromethyl)pyridine-2-carbonyl]amino]-1H-benzimidazole-5-carboxylate (400 mg, 839 umol) in THF (5 mL) was added MeMgBr (3 M, 2.80 mL) at 0° C. and the reaction mixture was stirred at 0° C. for 1 hr. On completion, the reaction mixture was quenched by saturated NH₄Cl (5 mL) and extracted with DCM/MeOH=10:1 (3×40 mL). The combined organic layers were washed with brine (2×30 mL), dried over Na₂SO₄, filtered and concentrated in vacuo. The residue was purified by silica gel chromatography (DCM:MeOH=30:1) to give the title compound (250 mg, 62% yield) as yellow solid. ¹H NMR (400 MHz, DMSO-d₆) δ 12.40 (s, 1H), 8.56 (s, 1H), 8.46 (d, J=7.6 Hz, 1H), 8.36 (t, J=7.6 Hz, 1H), 8.15 (d, J=7.2 Hz, 1H), 7.44 (s, 1H), 5.86 (s, 1H), 4.45 (s, 1H), 3.29-3.25 (m, 2H), 2.81-2.70 (m, 1H), 2.13-2.00 (m, 2H), 1.94-1.80 (m, 2H), 1.67-1.51 (m, 8H), 1.49-1.37 (m, 1H), 1.12-0.97 (m, 2H).

### Step 11—N-[2-(4-formylcyclohexyl)-6-(1-hydroxy-1-methyl-ethyl)-3H-benzimidazol-5-yl]-6-(trifluoromethyl)pyridine-2-carboxamide

To a solution of N-[2-[4-(hydroxymethyl)cyclohexyl]-6-(1-hydroxy-1-methyl-ethyl)-3H-benzimidazol-5-yl]-6-(trifluoromethyl)pyridine-2-carboxamide (230 mg, 482 umol) in DCM (3 mL) was added DMP (266 mg, 627 umol) and the reaction mixture was stirred at 20° C. for 1 hr. On completion, the reaction mixture was quenched by sat. aq. Na₂S₂O₃ (1 mL), basified with sat. aq. NaHCO₃ until the pH=8 and extracted with DCM/MeOH=10:1 (3×25 mL). The combined organic layers were washed with brine (2×20 mL), dried over Na₂SO₄, filtered and concentrated in vacuo to give the title compound (220 mg, 96% yield) as yellow solid. ¹H NMR (400 MHz, DMSO-d₆) δ 12.52-12.23 (m, 1H), 12.15-11.94 (m, 1H), 8.61-8.53 (m, 1H), 8.51-8.42 (m, 1H), 8.40-8.32 (m, 1H), 8.15 (d, J=8.0 Hz, 1H), 7.55-7.30 (m, 1H), 5.86-5.83 (m, 1H), 2.82-2.68 (m, 1H), 2.15-1.90 (m, 4H), 1.60 (s, 6H), 1.56-1.34 (m, 3H), 1.25-1.10 (m, 2H).

### 5-(Trifluoromethyl)pyrazolo[1,5-a]pyrimidine-3-carboxamide (Intermediate BPE)

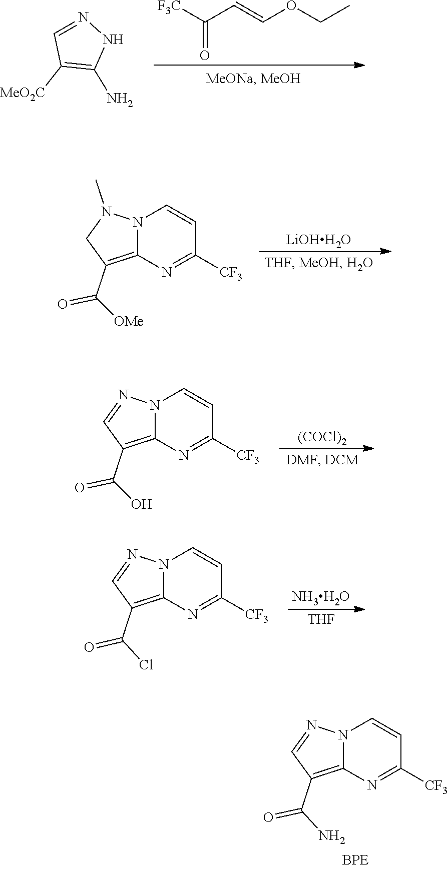

### Step 1—5-(Trifluoromethyl)pyrazolo[1,5-a]pyrimidine-3-carboxylate

A mixture of methyl 5-amino-1H-pyrazole-4-carboxylate (1.00 g, 7.09 mmol), 4-ethoxy-1,1,1-trifluoro-but-3-en-2-one (1.20 g, 7.16 mmol, CAS #17129-06-5) and NaOMe (400 mg, 7.40 mmol) in MeOH (20 mL) as stirred at 25° C. for 1 hour. Then, the mixture was stirred at 60° C. for 15 hours. On completion, the mixture was concentrated in vacuo. The residue was diluted with DCM (30 mL), washed with brine (10 mL), dried over Na₂SO₄, filtered and concentrated in vacuo to give the title compound (1.10 g, 63% yield) as light yellow solid. ¹H NMR (400 MHz, CDCl₃) δ 8.96 (d, J=7.2 Hz, 1H), 8.74 (s, 1H), 7.34 (d, J=7.2 Hz, 1H), 3.99 (s, 3H).

### Step 2—5-(Trifluoromethyl)pyrazolo[1,5-a]pyrimidine-3-carboxylic acid

To a solution of methyl 5-(trifluoromethyl)pyrazolo[1,5-a]pyrimidine-3-carboxylate (1.10 g, 4.49 mmol) in THF (9 mL) and MeOH (3 mL) was added a solution of LiOH·H₂O (378 mg, 9.01 mmol) in H₂O (3 mL) at 20° C. The mixture was stirred at 50° C. for 1 hour. On completion, after cooled to 25° C., and the mixture was acidified to pH=4-5 with 1.0 M aq. HCl. The mixture was concentrated in vacuo and the residue was purified by reversed phase flash (FA condition) to give the title compound (800 mg, 77% yield) as light yellow solid. ¹H NMR (400 MHz, DMSO-d₆) δ 9.60 (d, J=7.2 Hz, 1H), 8.80 (s, 1H), 7.71 (d, J=7.2 Hz, 1H).

### Step 3—5-(Trifluoromethyl)pyrazolo[1,5-a]pyrimidine-3-carbonyl chloride

To a solution of 5-(trifluoromethyl)pyrazolo[1,5-a]pyrimidine-3-carboxylic acid (800 mg, 3.46 mmol) and DMF (0.05 mL) in DCM (10 mL) was added (COCl)₂ (884 mg, 6.97 mmol) at 0° C. slowly. The mixture was stirred at 25° C. for 1 hour. On completion, the mixture was concentrated in vacuo to give the title compound (860 mg, 99% yield) as light yellow gum.

### Step 4—5-(Trifluoromethyl)pyrazolo[1,5-a]pyrimidine-3-carboxamide

To NH₃·H₂O (10 mL) was added a solution of 5-(trifluoromethyl)pyrazolo[1,5-a]pyrimidine-3-carbonyl chloride (860 mg, 3.45 mmol) in THF (10 mL) at 0° C. and the mixture was stirred at 0° C. for 0.5 hour. On completion, the mixture was extracted with EA (3×20 mL). The combine organic layer was washed with brine (10 mL), dried over Na₂SO₄, filtered and concentrated in vacuo. The residue was purified by column chromatography on silica gel (PE:EA=5:1-1:1) to give the title compound (370 mg, 46% yield) as light yellow solid. ¹H NMR (400 MHz, DMSO-d₆) δ 9.63 (d, J=7.2 Hz, 1H), 8.78 (s, 1H), 7.70 (d, J=7.2 Hz, 1H), 7.64 (s, 1H), 7.28 (s, 1H).

### N-[2-(4-formylcyclohexyl)-6-methoxy-indazol-5-yl]-5-(trifluoromethyl)pyrazolo[1,5-a]pyrimidine-3-carboxamide (Intermediate BPF)

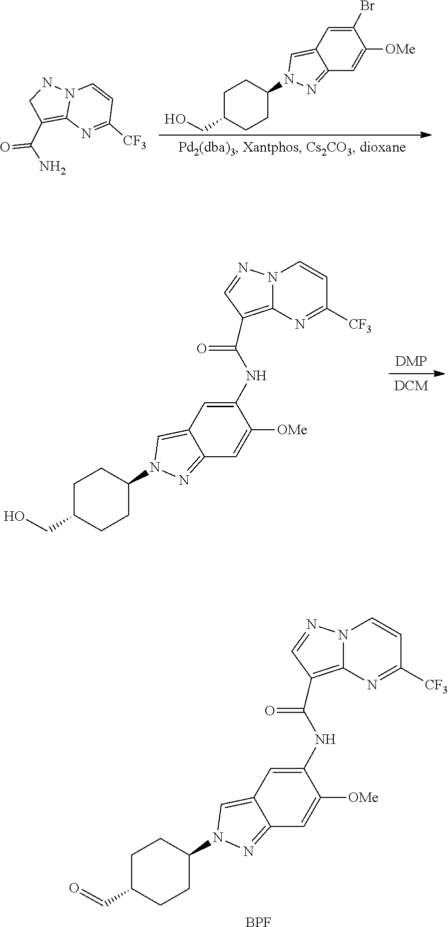

### Step 1—N-[2-[4-(hydroxymethyl)cyclohexyl]-6-methoxy-indazol-5-yl]-5-(trifluoromethyl)pyrazolo [1,5-a]pyrimidine-3-carboxamide

A mixture of 5-(trifluoromethyl)pyrazolo[1,5-a]pyrimidine-3-carboxamide (320 mg, 1.39 mmol, Intermediate BPE), [4-(5-bromo-6-methoxy-indazol-2-yl)cyclohexyl]methanol (400 mg, 1.18 mmol, synthesized via Steps 1-3 of Intermediate ATE), Pd₂(dba)₃ (109 mg, 119 umol), Xantphos (140 mg, 241 umol) and Cs₂CO₃ (768 mg, 2.36 mmol) in dioxane (10 mL) was stirred at 80° C. for 16 hours under N₂. On completion, after the reaction mixture was cooled to 25° C., the mixture was filtered and the filter cake was washed with THF (20 mL). The filtrate and washing were combined and concentrated in vacuo. The residue was purified by reversed phase flash (FA condition) and prep-HPLC (column: Phenomenex Synergi C18 150*25*10 um; mobile phase: [water(0.225% FA)-ACN]; B %: 32%-62%, 10 min) to give the title compound (80.0 mg, 13% yield) as light yellow solid. LC-MS (ESI⁺) m/z 489.3 (M+H)⁺.

### Step 2—N-[2-(4-formylcyclohexyl)-6-methoxy-indazol-5-yl]-5-(trifluoromethyl)pyrazolo[1,5-a] pyrimidine-3-carboxamide

To a solution of N-[2-[4-(hydroxymethyl)cyclohexyl]-6-methoxy-indazol-5-yl]-5-(trifluoromethyl) pyrazolo[1,5-a]pyrimidine-3-carboxamide (70.0 mg, 143 umol) in DCM (3 mL) was added Dess-Martin (70.0 mg, 165 umol) at 15° C. and the mixture was stirred at 15° C. for 2 hours. On completion, the reaction was quenched with sat. aq. Na₂S₂O₃ (5 mL) and the mixture was partitioned, and the aqueous phase was extracted with DCM (2×10 mL). The combined organic layer was washed with sat. aq. NaHCO₃ (5 mL) and brine (5 mL), dried over Na₂SO₄, filtered and concentrated in vacuo to give the title compound (68.0 mg, 98% yield) as light yellow solid. LC-MS (ESI⁺) m/z 487.1 (M+H)⁺.

### N-(2-((1R,4R)-4-formylcyclohexyl)-6-methoxy-2H-indazol-5-yl)-5-(trifluoromethyl)picolinamide (Intermediate BPG)

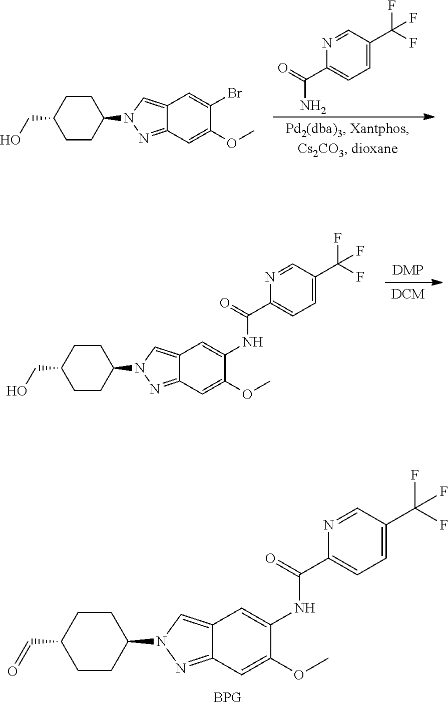 [SMILES: COc1cc2nn([C@H]3CC[C@H](CO)CC3)cc2cc1Br ; NC(=O)c1ccc(C(F)(F)F)cn1 ; COc1cc2nn([C@H]3CC[C@H](CO)CC3)cc2cc1NC(=O)c1ccc(C(F)(F)F)cn1 ; COc1cc2nn([C@H]3CC[C@H](C=O)CC3)cc2cc1NC(=O)c1ccc(C(F)(F)F)cn1]

### Step 1—N-(2-((1R,4R)-4-(hydroxymethyl)cyclohexyl)-6-methoxy-2H-indazol-5-yl)-5-(trifluoromethyl) picolinamide

To solution of [4-(5-bromo-6-methoxy-indazol-2-yl)cyclohexyl]methanol (313 mg, 922 umol, synthesized via Steps 1-3 of Intermediate ATE) and 5-(trifluoromethyl)pyridine-2-carboxamide (175 mg, 922 umol, 22245-86-9) in the dioxane (10 mL) was added Pd2(dba)₃ (84.5 mg, 92.2 umol), Xantphos (107 mg, 184 umol) and Cs2CO3 (601 mg, 1.85 mmol). The mixture was stirred at 100° C. for 24 hrs under N₂. On completion, the mixture was concentrated in vacuo and purified by column chromatography (SiO2, PE:EA=10:1 to 0:1) to give the title compound (360 mg, 87% yield) as yellow solid. ¹H NMR (400 MHz, CHLOROFORM-d) δ=10.68 (s, 1H), 8.99-8.92 (m, 1H), 8.86 (s, 1H), 8.45 (d, J=8.1 Hz, 1H), 8.16 (dd, J=1.8, 8.2 Hz, 1H), 7.89 (s, 1H), 7.09 (s, 1H), 4.33 (tt, J=3.8, 11.9 Hz, 1H), 4.04 (s, 3H), 3.57 (t, J=5.5 Hz, 2H), 2.37-2.29 (m, 2H), 2.06-1.93 (m, 4H), 1.74-1.65 (m, 1H), 1.47 (t, J=5.2 Hz, 1H), 1.29-1.21 (m, 2H).

### Step 2—N-(2-((1R,4R)-4-formylcyclohexyl)-6-methoxy-2H-indazol-5-yl)-5-(trifluoromethyl) picolinamide

To a solution of N-[2-[4-(hydroxymethyl)cyclohexyl]-6-methoxy-indazol-5-yl]-5-(trifluoromethyl) pyridine-2-carboxamide (60.0 mg, 134 umol) in the DCM (2 mL) was added DMP (68.1 mg, 160 umol) and the mixture was stirred at 25° C. for 1 hour. On completion, the reaction mixture was quenched with saturated Na₂S₂O₃ (aq, 5 mL) and NaHCO₃ (aq, 5 mL), then the mixture was extracted with DCM (2×20 mL). The organic layer was dried over Na₂SO₄, filtered and concentrated in vacuo to give the title compound (58.0 mg, 98% yield) as yellow solid. LC-MS (ESI⁺) m/z 447.1 (M+H)⁺.

### [1-[(4-Methoxyphenyl) methyl]-2,6-dioxo-3-piperidyl] trifluoromethanesulfonate (Intermediate IQ)

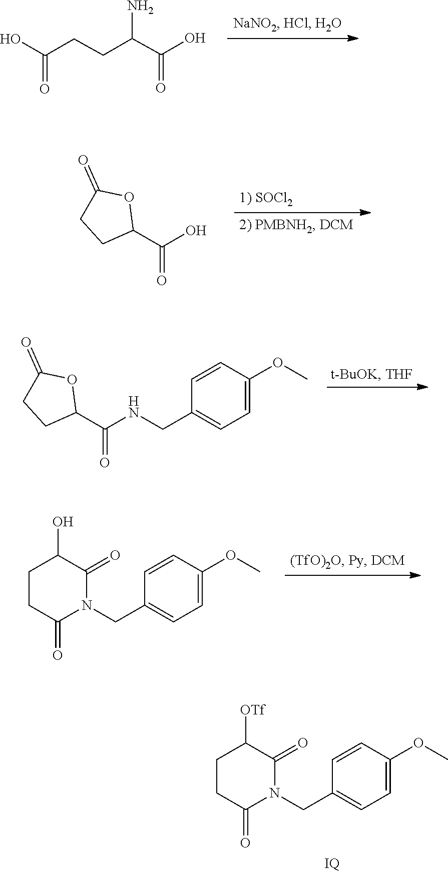

### Step 1—5-Oxotetrahydrofuran-2-carboxylic acid

To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at −5° C. The mixture was stirred at 15° C. for 12 hrs. On completion, the mixture was concentrated and then dissolved in EA (500 mL) and filtered and washed with EA (3×100 mL). The filtrate and washed solution were dried over Na₂SO₄, filtered and concentrated in vacuo to give the title compound (200 g, crude) as yellow oil. ¹H NMR (400 MHz, CDCl₃) δ 6.43 (s, 1H), 5.02-4.95 (m, 1H), 2.67-2.38 (m, 4H)

### Step 2—N-[(4-methoxyphenyl)methyl]-5-oxo-tetrahydrofuran-2-carboxamide

To 5-oxotetrahydrofuran-2-carboxylic acid (120 g, 922 mmol) was added SOCl₂ (246 g, 2.07 mol) at 0° C. slowly. The mixture was stirred at 85° C. for 3 hrs, and then the mixture was stirred at 15° C. for 6 hrs. The mixture was concentrated in vacuo. The residue was dissolved in dry DCM (1 L) at 0° C. under N₂. After that a solution of Et₃N (187 g, 1.84 mol) and 4-methoxybenzylamine (101 g, 738 mmol) in DCM (400 mL) was added, then the mixture was stirred at 15° C. for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHz, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

### Step 3—3-Hydroxy-1-[(4-methoxyphenyl)methyl]piperidine-2,6-dione

A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

### Step 4—[1-[(4-Methoxyphenyl) methyl]-2,6-dioxo-3-piperidyl]trifluoromethanesulfonate

To a solution of 3-hydroxy-1-[(4-methoxyphenyl) methyl]piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N₂. On completion, the mixture was concentrated in vacuo. The residue was purified by column chromatography on silica gel (PE:EA=20:1/8:1) to give the title compound (45.0 g, 68% yield) as light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### 3-(4-Bromo-3-methyl-2-oxo-2,3-dihydro-1H-benzo[d]imidazol-1-yl)piperidine-2,6-dione (Intermediate HP)

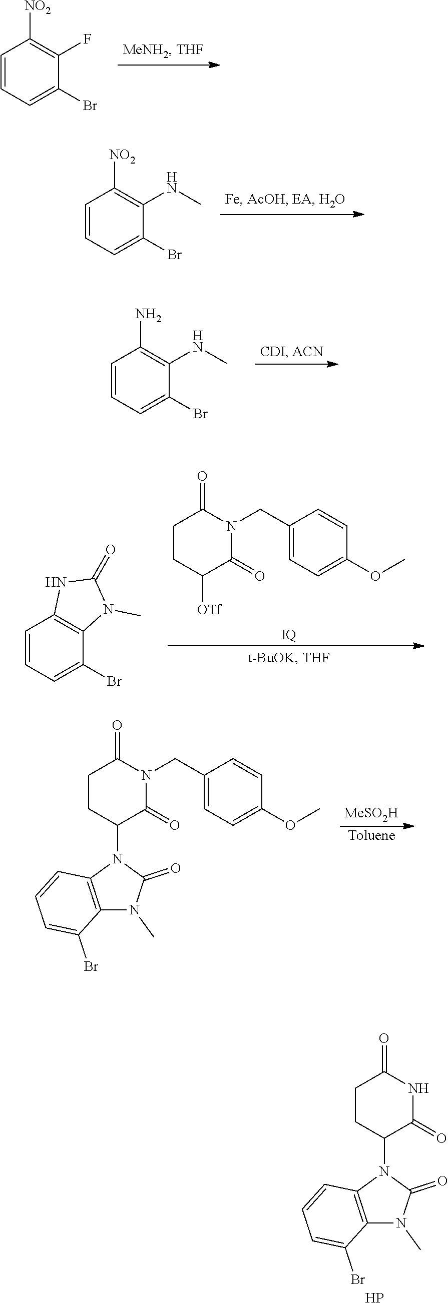

### Step 1—2-Bromo-N-methyl-6-nitro-aniline

To a solution of 1-bromo-2-fluoro-3-nitro-benzene (40.0 g, 181 mmol, CAS #58534-94-4) in THF (40 mL) was added MeNH₂ (2 M, 400 mL). The reaction mixture was stirred at 60° C. for 12 hours. On completion, the reaction mixture was poured into sat. NaHCO₃ (30 mL) and extracted with EA (3×200 mL). The combined organic layers were washed with brine (2×200 mL), dried with anhydrous Na₂SO₄, filtered and concentrated in vacuo to give the title compound (40.0 g, 95% yield) as red oil. LC-MS (ESI⁺) m/z 230.9 (M+H)⁺.

### Step 2—3-Bromo-N2-methyl-benzene-1,2-diamine

To a mixture of 2-bromo-N-methyl-6-nitro-aniline (23.0 g, 99.5 mmol) in EA (300 mL) and H₂O (10 mL) was added AcOH (100 mL). The mixture was warmed to 50° C. Then Fe (22.2 g, 398 mmol) was added to the reaction mixture and the mixture was heated to 80° C. about 4 hours. On completion, the reaction mixture was filtered and concentrated in vacuo. The residue was diluted with water (100 mL) and extracted with EA (3×200 mL). The combined organic layers was dried over Na₂SO₄, filtered and concentrated in vacuo to give the title compound (20.0 g, 99% yield) as red oil. ¹H NMR (400 MHz, DMSO-d₆) δ 6.73-6.70 (m, 1H), 6.68-6.60 (m, 2H), 5.02 (s, 2H), 3.67 (s, 1H), 2.58 (s, 3H).

### Step 3—4-Bromo-3-methyl-1H-benzimidazol-2-one

To a mixture of 3-bromo-N2-methyl-benzene-1,2-diamine (20.0 g, 99.4 mmol) in ACN (300 mL) was added CDI (32.2 g, 198 mmol). The reaction mixture was stirred at 85° C. for 12 hours under N₂ atmosphere. On completion, the reaction mixture was concentrated in vacuo. The reaction mixture was diluted with water (200 mL), where a solid precipitate was formed, which was filtered off. The solid was washed with water (1 L) and dried in vacuo to give the title compound (20.0 g, 88% yield) as white solid. ¹H NMR (400 MHz, DMSO-d₆) δ 11.17 (s, 1H), 7.14 (dd, J=1.2, 8.0 Hz, 1H), 7.00-6.95 (m, 1H), 6.93-6.87 (m, 1H), 3.55 (s, 3H).

### Step 4—3-(4-Bromo-3-methyl-2-oxo-benzimidazol-1-yl)-1-[(4-methoxyphenyl)methyl]piperidine-2,6-dione

To a solution of 4-bromo-3-methyl-1H-benzimidazol-2-one (12.0 g, 52.8 mmol) in THF (300 mL) was added t-BuOK (7.12 g, 63.4 mmol). The reaction mixture was stirred at 0° C. for 0.5 hr. Subsequently, [1-[(4-methoxyphenyl)methyl]-2,6-dioxo-3-piperidyl]trifluoromethanesulfonate (20.1 g, 52.8 mmol, Intermediate IQ) in a solution of THF (100 mL) was added dropwise. The resulting reaction mixture was stirred at 20° C. for 0.5 hr under N₂. On completion, the reaction mixture was quenched with saturated NH₄Cl (100 mL), and extracted with ethyl acetate (200 mL). The combined organic layers were washed with brine (2×100 mL), dried over anhydrous sodium sulfate, filtered, the filtrate was concentrated in vacuo. The crude product was purified by reversed-phase HPLC (0.1% FA condition) to give the title compound (13.3 g, 55% yield) as a yellow solid. ¹H NMR (400 MHz, CDCl₃) δ 7.38 (d, J=8.8 Hz, 2H), 7.22 (d, J=8.0 Hz, 1H), 6.84 (d, J=8.8 Hz, 2H), 6.80 (t, J=8.0 Hz, 1H), 6.48-6.40 (d, J=8.0 Hz, 1H), 5.22 (dd, J=5.2, 12.8 Hz, 1H), 5.04-4.93 (m, 2H), 3.81 (s, 3H), 3.80 (s, 3H), 3.12-2.98 (m, 1H), 2.93-2.77 (m, 1H), 2.62 (dq, J=4.4, 13.2 Hz, 1H), 2.20-2.17 (m, 1H).

### Step 5—3-(4-bromo-3-methyl-2-oxo-2,3-dihydro-1H-benzo[d]imidazol-1-yl)piperidine-2,6-dione

A mixture of 3-(4-bromo-3-methyl-2-oxo-benzimidazol-1-yl)-1-[(4-methoxyphenyl)methyl]piperidine-2,6-dione (13.3 g, 29.0 mmol) in a mixed solvent of Tol. (80 mL) and methane sulfonic acid (40 mL) was degassed and purged with N₂ for 3 times, and then the mixture was stirred at 120° C. for 2 hrs under N₂ atmosphere. On completion, the reaction mixture was concentrated in vacuo to remove toluene. The residue was added 200 mL of ice water, and then white solid precipitate formed. The mixture was filtered and the filtered cake was collected and dried over in vacuo to give the title compound (7.30 g, 74% yield) as white solid. ¹H NMR (400 MHz, DMSO-d₆) δ 11.13 (s, 1H), 7.25 (d, J=8.0 Hz, 1H), 7.17 (d, J=8.0 Hz, 1H), 7.05-6.93 (m, 1H), 5.41 (dd, J=5.2, 12.8 Hz, 1H), 3.64 (s, 3H), 2.96-2.83 (m, 1H), 2.78-2.59 (m, 2H), 2.08-2.00 (m, 1H).

### 3-(3-Methyl-2-oxo-4-(piperidin-4-yl)-2,3-dihydro-1H-benzo[d]imidazol-1-yl)piperidine-2,6-dione (Intermediate AZK)

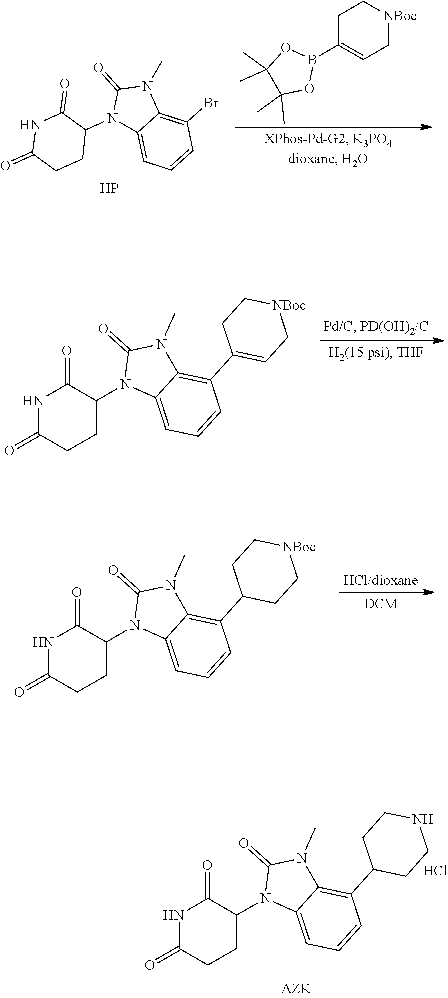

### Step 1—Tert-butyl 4-(1-(2,6-dioxopiperidin-3-yl)-3-methyl-2-oxo-2,3-dihydro-1H-benzo[d]imidazol-4-yl)-5,6-dihydropyridine-1(2H)-carboxylate

To a solution of 3-(4-bromo-3-methyl-2-oxo-benzimidazol-1-yl)piperidine-2,6-dione (9.00 g, 26.6 mmol, Intermediate HP), tert-butyl 4-(4,4,5,5-tetramethyl-1,3,2-dioxaborolan-2-yl)-3,6-dihydro-2H-pyridine-1-carboxylate (12.3 g, 39.9 mmol) and XPhos-Pd-G2 (2.09 g, 2.66 mmol) in dioxane (150 mL) and H₂O (15 mL) was added K₃PO₄ (11.3 g, 53.2 mmol). The reaction mixture was stirred at 80° C. for 4 hours under N₂. On completion, the reaction mixture was filtered. The filtrate was dried with anhydrous Na₂SO₄ and filtered. The filtrate was concentrated in vacuo. The residue was triturated with sat. NH₄Cl (2×50 mL), water (2×50 mL) and EA (2×50 mL) and filtered. The solid was dried in vacuo to give the title compound (8.00 g, 68% yield) as an off-white solid. LC-MS (ESI⁺) m/z 441.1 (M+H)⁺

### Step 2—Tert-butyl 4-(1-(2,6-dioxopiperidin-3-yl)-3-methyl-2-oxo-2,3-dihydro-1H-benzo[d]imidazol-4-yl)piperidine-1-carboxylate

To a solution of tert-butyl 4-[1-(2,6-dioxo-3-piperidyl)-3-methyl-2-oxo-benzimidazol-4-yl]-3,6-dihydro-2H-pyridine-1-carboxylate (8.00 g, 18.2 mmol) in DMF (20 mL) and THF (60 mL) was added H₂, Pd/C (1.00 g, 10 wt %) and Pd(OH)₂ (1.00 g, 3.56 mmol, 50 wt %). The mixture was degassed and purged with nitrogen 3 times, then degassed and purged with hydrogen 3 times. The mixture was stirred at 25° C. for 16 hrs under hydrogen (15 psi) atmosphere. On completion, the reaction mixture was filtered and the combined filtrates were concentrated in vacuo to give the title compound (5.60 g, 70% yield) as a white solid. ¹H NMR (400 MHz, DMSO-d₆) δ 11.10 (s, 1H), 7.06-6.92 (m, 3H), 5.38 (m, 1H), 4.18-3.96 (m, 2H), 3.60 (s, 3H), 3.48-3.39 (m, 1H), 2.97-2.81 (m, 3H), 2.76-2.61 (m, 2H), 2.05-1.94 (m, 1H), 1.81 (m, 2H), 1.65-1.50 (m, 2H), 1.47-1.40 (m, 9H). LC-MS (ESI⁺) m/z 287.4 (387.3)⁺.

### Step 3—3-(3-Methyl-2-oxo-4-(piperidin-4-yl)-2,3-dihydro-1H-benzo[d]imidazol-1-yl)piperidine-2,6-dione

To a solution of tert-butyl 4-[1-(2,6-dioxo-3-piperidyl)-3-methyl-2-oxo-benzimidazol-4-yl]piperidine-1-carboxylate (100 mg, 226 umol) in DCM (1 mL) was added HCl/dioxane (1 mL). The reaction mixture was stirred at 25° C. for 0.5 hr. On completion, the reaction mixture was concentrated in vacuo to give the title compound (85.0 mg, 99% yield) as a yellow solid. LC-MS (ESI⁺) m/z 343.3 (M+H)⁺.

### 1-(2,6-dioxo-3-piperidyl)-3-methyl-2-oxo-benzimidazole-4-carbaldehyde (Intermediate WW)

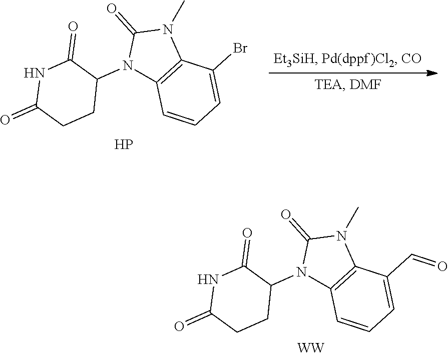

To a solution of 3-(4-bromo-3-methyl-2-oxo-2,3-dihydro-1H-benzo[d]imidazol-1-yl)piperidine-2,6-dione (500 mg, 1.48 mmol, Intermediate HP) in DMF (20 mL) was added TEA (448 mg, 4.44 mmol), Pd(dppf)Cl₂ (162 mg, 221 umol) and Et₃SiH (515 mg, 4.44 mmol). The reaction mixture was stirred at 80° C. for 16 hours under CO (50 Psi). On completion, the reaction mixture was concentrated in vacuo and purified by reverse phase (0.1% FA) to give the title compound (400 mg, 47% yield) as a white solid. LC-MS (ESI⁺) m/z 288.0 (M+H)⁺.

### 3-[3-Methyl-4-[[4-(methylamino)-1-piperidyl]methyl]-2-oxo-benzimidazol-1-yl]piperidine-2,6-dione (Intermediate WX)

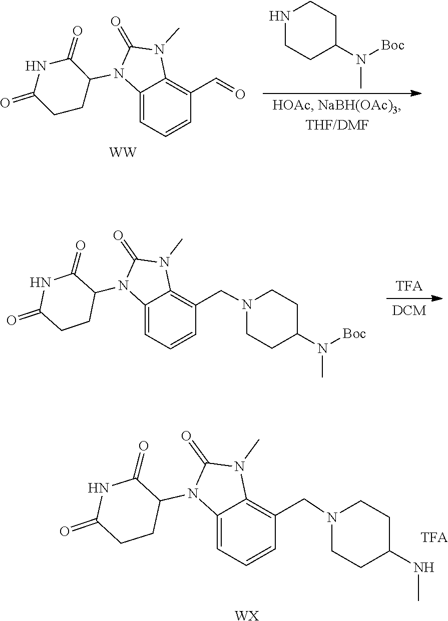

### Step 1—Tert-butyl N-[1-[[1-(2,6-dioxo-3-piperidyl)-3-methyl-2-oxo-benzimidazol-4-yl]methyl]-4-piperidyl]-N-methyl-carbamate

To a solution of 1-(2,6-dioxo-3-piperidyl)-3-methyl-2-oxo-benzimidazole-4-carbaldehyde (160 mg, 556 umol, Intermediate WW) and tert-butyl N-methyl-N-(4-piperidyl)carbamate (119 mg, 556 umol) in a mixed solvents of THF (3 mL) and DMF (1.5 mL) was added AcOH until the pH=5-7. After the reaction mixture was stirred at 20° C. for 3 hours. NaBH(OAc)₃ (177 mg, 835 umol) was added to the reaction mixture. The mixture was stirred at 20° C. for 12 hours. On completion, the reaction mixture was quenched by water (3 drops) and filtered. The filtrate was concentrated in vacuo. The residue was purified by reverse phase (FA condition) to give the title compound (220 mg, 46% yield) as white solid. LC-MS (ESI⁺) m/z 486.2 (M+H)⁺.

### Step 2—3-[3-Methyl-4-[[4-(methylamino)-1-piperidyl]methyl]-2-oxo-benzimidazol-1-yl]piperidine-2,6-dione

To a solution of tert-butyl N-[1-[[1-(2,6-dioxo-3-piperidyl)-3-methyl-2-oxo-benzimidazol-4-yl]methyl]-4-piperidyl]-N-methyl-carbamate (200 mg, 235 umol) in DCM (3 mL) was added TFA (3 mL). The reaction mixture was stirred at 15° C. for 0.5 hour. On completion, the reaction mixture was concentrated in vacuo to give the title compound (220 mg, 100% yield, TFA salt) as yellow oil. LC-MS (ESI⁺) m/z 386.2 (M+H)⁺.

### Tert-butyl 6-(2-aminoethyl)-2-azaspiro[3.3]heptane-2-carboxylate (Intermediate ATG)

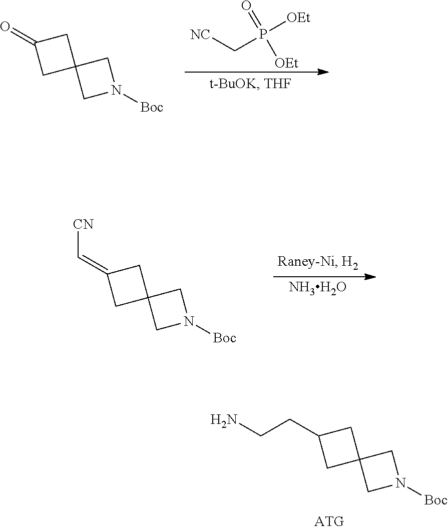 [SMILES: CC(C)(C)OC(=O)N1CC2(CC(=O)C2)C1 ; CCOP(=O)(CC#N)OCC ; CC(C)(C)OC(=O)N1CC2(CC(=CC#N)C2)C1 ; "NH3·H2O" ; CC(C)(C)OC(=O)N1CC2(CC(CCN)C2)C1]

### Step 1—Tert-butyl 6-(cyanomethylene)-2-azaspiro[3.3]heptane-2-carboxylate

To a solution of t-BuOK (3.98 g, 35.5 mmol,) in THF (35 mL) was added a solution of 2-diethoxyphosphorylacetonitrile (6.29 g, 35.5 mmol) in THF (70 mL) at 0° C. dropwise, and the reaction was stirred at 25° C. for 0.5 h. After, the mixture was cooled to 0° C. and a solution of tert-butyl 6-oxo-2-azaspiro[3.3]heptane-2-carboxylate (5.00 g, 23.7 mmol, CAS #1147557-97-8) in THF (35 mL) was added and the reaction was stirred at 25° C. for 16 hours. On completion, the reaction was quenched with water (10 mL) and the solvent was removed in vacuo to give a residue. The residue was purified by silica gel column chromatography (PE:EA from 5:1 to 1:1) to give the title compound (4.10 g, 66% yield) as a yellow oil. ¹H NMR (400 MHz, DMSO-d₆) δ 5.55 (t, J=2.4 Hz, 1H), 3.91 (d, J=2.0 Hz, 4H), 3.17-3.01 (m, 4H), 1.37 (s, 9H).

### Step 2—Tert-butyl 6-(2-aminoethyl)-2-azaspiro[3.3]heptane-2-carboxylate

To a solution of tert-butyl 6-(cyanomethylene)-2-azaspiro[3.3]heptane-2-carboxylate (4.10 g, 17.5 mmol) in MeOH (80 mL) and NH₃·H₂O (8 mL) was added Raney-Ni (1.50 g, 17.5 mmol). The mixture was degassed and purged with H₂ gas 3 times and then was stirred at 25° C. under H₂ at 50 psi for 3 hours. On completion, the reaction was filtered through celite, the filtered cake was washed with MeOH (3×5 mL) and the filtrate was concentrated in vacuo to give the title compound (3.10 g, 66% yield) as yellow oil. ¹H NMR (400 MHz, DMSO-d₆) δ 3.82 (d, J=7.6 Hz, 4H), 2.47-2.00 (m, 5H), 1.79-1.67 (m, 2H), 1.46-1.38 (m, 2H), 1.36 (s, 9H).

### 2-(2,6-Dioxopiperidin-3-yl)-4-fluoroisoindoline-1,3-dione (Intermediate R)

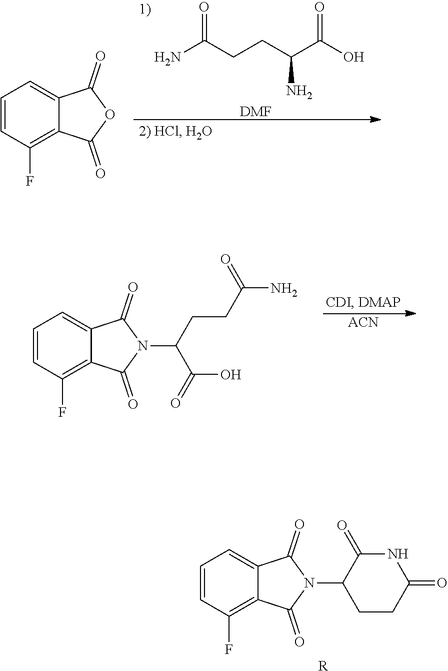

### Step 1—5-amino-2-(4-fluoro-1,3-dioxoisoindolin-2-yl)-5-oxopentanoic acid

To a stirred solution of 4-fluoroisobenzofuran-1,3-dione (25 g, 150 mmol, CAS #652-39-1) in DMF (100 mL) was added L-glutamine (22 g, 150 mmol) at rt. The resulting reaction mixture was heated to at 90° C. and stirred for 2 h. The reaction mixture was then evaporated under reduced pressure, transferred into 4 N aqueous HCl solution and the resulting mixture was stirred for 36 h at rt. The solid precipitate was then filtered off, washed with cold water and dried under reduced pressure to give 5-amino-2-(4-fluoro-1,3-dioxoisoindolin-2-yl)-5-oxopentanoic acid as a white solid (28 g, 63%). LC-MS (ESI⁺) m/z 295 (M+H)⁺.

### Step 2—2-(2,6-dioxopiperidin-3-yl)-4-fluoroisoindoline-1,3-dione

To a stirred solution of 5-amino-2-(4-fluoro-1,3-dioxoisoindolin-2-yl)-5-oxopentanoic acid (28 g, 95 mmol) in acetonitrile (200 mL) was added CDI (19 g, 110 mmol) and DMAP (0.14 g, 1.1 mmol) at rt. The resulting reaction mixture then heated to 90° C. and stirred for 5 h. The reaction mixture was then evaporated under reduced pressure. The crude product was purified using silica gel column chromatography (2% MeOH-DCM) to give 2-(2,6-dioxopiperidin-3-yl)-4-fluoroisoindoline-1,3-dione as a yellow solid (12 g, 46%). ¹H NMR (400 MHz, DMSO) δ ppm 11.16 (s, 1H), 7.98-7.93 (m, 1H), 7.80-7.76 (m, 2H), 5.19-5.14 (m, 1H), 2.94-2.85 (m, 1H), 2.63-2.54 (m, 2H), 2.09-2.04 (m, 1H).

### 4-[2-(2-Azaspiro[3.3]heptan-6-yl)ethylamino]-2-(2,6-dioxo-3-piperidyl)isoindoline-1,3-dione (Intermediate ATH)

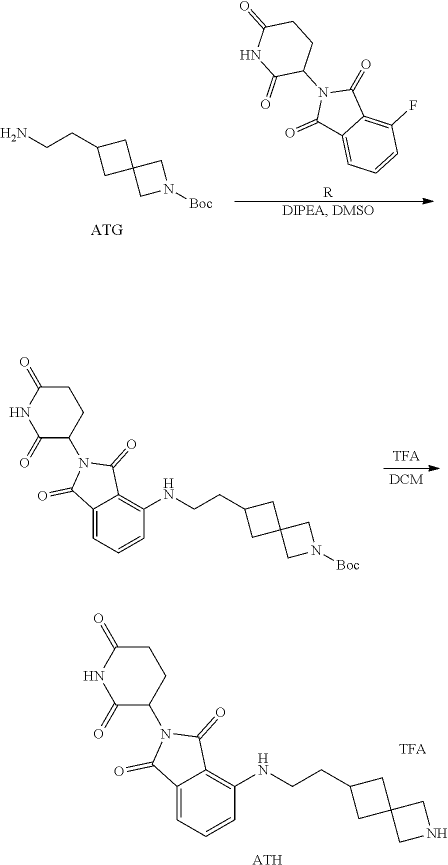

### Step 1—Tert-butyl 6-[2-[[2-(2,6-dioxo-3-piperidyl)-1,3-dioxo-isoindolin-4-yl]amino]ethyl]-2-azaspiro [3.3]heptane-2-carboxylate

To a solution of tert-butyl 6-(2-aminoethyl)-2-azaspiro[3.3]heptane-2-carboxylate (3.00 g, 12.5 mmol, Intermediate ATG) and 2-(2,6-dioxo-3-piperidyl)-4-fluoro-isoindoline-1,3-dione (3.79 g, 13.7 mmol, Intermediate R) in DMSO (30 mL) was added DIPEA (4.84 g, 37.5 mmol). The mixture was stirred at 130° C. for 1 hour. On completion, the reaction was diluted with EA (150 mL), washed with water (3×50 mL) and brine (100 mL), dried over Na₂SO₄, filtered and concentrated in vacuo to give a crude product which was purified by reversed phase (0.1% FA condition) to give the title compound (3.20 g, 46% yield) as a yellow solid. ¹H NMR (400 MHz, DMSO-d₆) δ 11.10 (s, 1H), 7.59 (dd, J=7.2, 8.4 Hz, 1H), 7.11-6.97 (m, 2H), 6.49 (t, J=5.6 Hz, 1H), 5.06 (dd, J=5.6, 12.8 Hz, 1H), 3.84 (s, 2H), 3.73 (s, 2H), 3.22 (q, J=6.4 Hz, 2H), 2.91-2.83 (m, 1H), 2.65-2.54 (m, 2H), 2.32-2.22 (m, 2H), 2.16 (t, J=7.6 Hz, 1H), 2.04 (d, J=2.4 Hz, 1H), 1.86-1.78 (m, 2H), 1.65 (q, J=7.2 Hz, 2H), 1.36 (s, 9H); LC-MS (ESI⁺) m/z 497.3 (M+H)⁺.

### Step 2—4-[2-(2-Azaspiro[3.3]heptan-6-yl)ethylamino]-2-(2,6-dioxo-3-piperidyl)isoindoline-1,3-dione

To a solution of tert-butyl 6-[2-[[2-(2,6-dioxo-3-piperidyl)-1,3-dioxo-isoindolin-4-yl]amino]ethyl]-2-azaspiro[3.3]heptane-2-carboxylate (0.30 g, 604 umol) in DCM (3 mL) was added TFA (2.31 g, 20.3 mmol). The mixture was stirred at 25° C. for 1 hour. On completion, the reaction was concentrated in vacuo to give the title compound (0.18 g, TFA, 58% yield) as a yellow solid. LC-MS (ESI⁺) m/z 397.2 (M+H)⁺.

### Tert-butyl 2-(aminomethyl)-7-azaspiro[3.5]nonane-7-carboxylate (Intermediate AJZ)

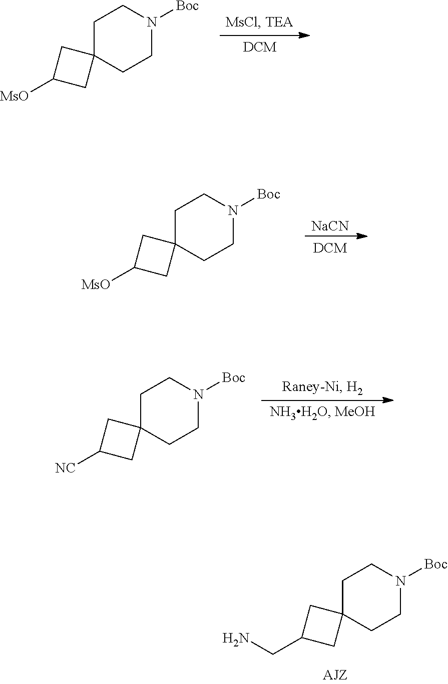

### Step 1—Tert-butyl 2-methylsulfonyloxy-7-azaspiro[3.5]nonane-7-carboxylate

To a solution of tert-butyl 2-hydroxy-7-azaspiro[3.5]nonane-7-carboxylate (2.00 g, 8.29 mmol, CAS #240401-28-9) and TEA (2.10 g, 20.7 mmol) in DCM (30 mL) was added MsCl (1.14 g, 9.95 mmol) dropwise at 0° C. Then the reaction mixture was stirred at 20° C. for 2 hrs. On completion, the mixture was quenched with water (50 mL), then extracted with DCM (2×50 mL). The organic layer was washed with citric acid (100 ml), brine (2×100 mL), dried with Na₂SO₄, filtered and the filtrate was concentrated in vacuo to give the title compound (2.60 g, 98% yield) as a yellowish oil. ¹H NMR (400 MHz, CDCl₃) δ 5.04 (t, J=7.2 Hz, 1H), 3.38-3.28 (m, 4H), 2.99 (s, 3H), 2.48-2.36 (m, 2H), 2.14-2.04 (m, 2H), 1.58-1.51 (m, 4H), 1.45 (s, 9H).

### Step 2—Tert-butyl 2-cyano-7-azaspiro[3.5]nonane-7-carboxylate

To a solution of tert-butyl 2-methylsulfonyloxy-7-azaspiro[3.5]nonane-7-carboxylate (2.60 g, 8.14 mmol) in DMF (20 mL) was added NaCN (598 mg, 12.2 mmol). The reaction mixture was stirred at 120° C. for 3 days. On completion, the reaction mixture was cooled to 25° C., diluted with water (100 mL), then extracted with EA (2×100 mL). The organic layer was washed with brine (2×100 mL) and concentrated in vacuo. The residue was purified by silica gel chromatography to give the title compound (1.32 g, 65% yield) as a white solid. ¹H NMR (400 MHz, CDCl₃) δ 3.36-3.29 (m, 4H), 3.13-3.02 (m, 1H), 2.30-2.14 (m, 4H), 1.66-1.62 (m, 2H), 1.58-1.53 (m, 2H), 1.45 (s, 9H).

### Step 3—Tert-butyl 2-(aminomethyl)-7-azaspiro[3.5]nonane-7-carboxylate

To a solution of tert-butyl 2-cyano-7-azaspiro[3.5]nonane-7-carboxylate (200 mg, 799 umol) and NH₃·H₂O (0.2 mL) in MeOH (5 mL) was added Raney-Ni (30 mg). The reaction mixture was stirred at 20° C. for 16 hrs under H₂ (15 Psi) atmosphere. On completion, the mixture was filtered and the filtrate was concentrated in vacuo to give the title compound (200 mg, 98% yield) as a white solid. ¹H NMR (400 MHz, CDCl₃) δ 3.37-3.32 (m, 2H), 3.30-3.23 (m, 2H), 2.70 (d, J=7.2 Hz, 2H), 2.33-2.24 (m, 1H), 1.97-1.88 (m, 2H), 1.59-1.55 (m, 2H), 1.45 (s, 9H), 1.44-1.37 (m, 4H).

### 4-(7-azaspiro[3.5]nonan-2-ylmethylamino)-2-(2,6-dioxo-3-piperidyl)isoindoline-1,3-dione (Intermediate AJF)

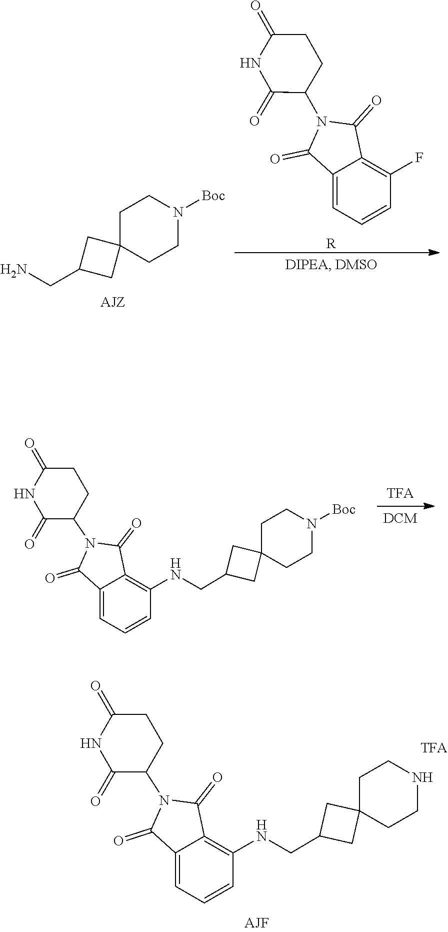

### Step 1—Tert-butyl 2-[[[2-(2,6-dioxo-3-piperidyl)-1,3-dioxo-isoindolin-4-yl]amino]methyl]-7-azaspiro [3.5]nonane-7-carboxylate

To a solution of tert-butyl 2-(aminomethyl)-7-azaspiro[3.5]nonane-7-carboxylate (200 mg, 786 umol, Intermediate AJZ) and 2-(2,6-dioxo-3-piperidyl)-4-fluoro-isoindoline-1,3-dione (228 mg, 825 umol, Intermediate R) in DMSO (3 mL) was added DIPEA (254 mg, 1.97 mmol). The reaction mixture was stirred at 125° C. for 3 hrs. On completion, the reaction mixture was diluted with water (50 mL), then extracted with EA (50 mL). The organic layer was concentrated in vacuo. The residue was purified by reverse phase (0.1% FA condition) to give the title compound (260 mg, 650% yield) as a yellow solid. LC-MS (ESI⁺) m/z 511.3 (M+H)⁺.

### Step 2—4-(7-Azaspiro[3.5]nonan-2-ylmethylamino)-2-(2,6-dioxo-3-piperidyl)isoindoline-1,3-dione

To a solution of tert-butyl 2-[[[2-(2,6-dioxo-3-piperidyl)-1,3-dioxo-isoindolin-4-yl]amino]methyl]-7-azaspiro[3.5]nonane-7-carboxylate (80.0 mg, 157 umol) in TFA (2 mL) was added DCM (2 mL). The reaction mixture was stirred at 20° C. for 2 hrs. On completion, the mixture was concentrated in vacuo to give the title compound (80 mg, 97% yield, TFA salt) as a yellow solid. LC-MS (ESI⁺) m/z 411.2 (M+H)⁺.

### Tert-butyl 2-(2-aminoethyl)-7-azaspiro[3.5]nonane-7-carboxylate (Intermediate ATB)

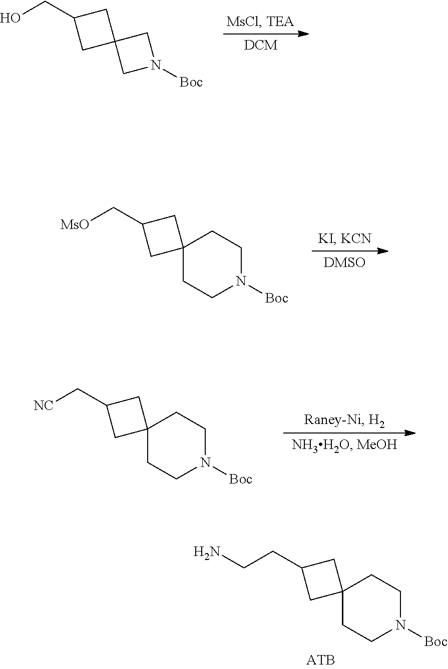 [SMILES: CC(C)(C)OC(=O)N1CC2(CC(CO)C2)C1 ; CC(C)(C)OC(=O)N1CCC2(CC1)CC(COS(C)(=O)=O)C2 ; CC(C)(C)OC(=O)N1CCC2(CC1)CC(CC#N)C2 ; CC(C)(C)OC(=O)N1CCC2(CC1)CC(CCN)C2]

### Step 1—Tert-butyl 2-(methylsulfonyloxymethyl)-7-azaspiro[3.5]nonane-7-carboxylate

To a solution of tert-butyl 2-(hydroxymethyl)-7-azaspiro[3.5]nonane-7-carboxylate (1.00 g, 3.92 mmol, CAS #1356476-27-1) and TEA (594 mg, 5.87 mmol) in DCM (15 mL) was added MsCl (538 mg, 4.70 mmol) at 0° C. The reaction mixture was stirred at 0˜20° C. for 1 hr. On completion, the reaction mixture was diluted with water (30 mL) and extracted with DCM (3×40 mL). The combined organic layers were washed with brine (2×30 mL), dried over Na₂SO₄, filtered and concentrated in vacuo to give the title compound (1.20 g, 91% yield) as yellow oil. ¹H NMR (400 MHz, CDCl₃) δ 4.20 (d, J=6.4 Hz, 2H), 3.41-3.32 (m, 2H), 3.30-3.23 (m, 2H), 3.02 (s, 3H), 2.75-2.62 (m, 1H), 2.03-1.93 (m, 2H), 1.66-1.61 (m, 2H), 1.61-1.59 (m, 1H), 1.58-1.56 (m, 1H), 1.50-1.47 (m, 2H), 1.45 (s, 9H).

### Step 2—Tert-butyl 2-(cyanomethyl)-7-azaspiro[3.5]nonane-7-carboxylate

To a solution of tert-butyl 2-(methylsulfonyloxymethyl)-7-azaspiro[3.5]nonane-7-carboxylate (1.20 g, 3.60 mmol) and KI (896 mg, 5.40 mmol) in DMSO (15 mL) was added KCN (257 mg, 3.96 mmol,) at 25° C. The reaction mixture was stirred at 100° C. for 4 hrs. On completion, the reaction mixture was diluted with water (100 mL) and extracted with EA (3×60 mL). The combined organic layers were washed with brine (2×30 mL), dried over Na₂SO₄, filtered and concentrated in vacuo. The residue was purified by silica gel chromatography (PE:EA=10:1) to give the title compound (510 mg, 53% yield) as yellow solid. ¹H NMR (400 MHz, CDCl₃) δ 3.43-3.33 (m, 2H), 3.31-3.22 (m, 2H), 2.70-2.52 (m, 1H), 2.45 (d, J=6.4 Hz, 2H), 2.14-1.99 (m, 2H), 1.68-1.58 (m, 4H), 1.54-1.48 (m, 2H), 1.46 (s, 9H).

### Step 3—Tert-butyl 2-(2-aminoethyl)-7-azaspiro[3.5]nonane-7-carboxylate

To a solution of tert-butyl 2-(cyanomethyl)-7-azaspiro[3.5]nonane-7-carboxylate (0.50 g, 1.89 mmol) and NH₃·H₂O (910 mg, 7.27 mmol, 28% solution) in MeOH (10 mL) was added Raney-Ni (32.4 mg, 378 umol). The reaction mixture was stirred at 20° C. for 3 hrs under hydrogen (50 psi). On completion, the reaction mixture was filtered and the filter cake was washed with methanol (50 mL). The combined organic phase was concentrated in vacuo to give the title compound (400 mg, 78% yield) as colorless oil. ¹H NMR (400 MHz, DMSO-d₆) δ 3.25-3.23 (m, 2H), 3.17-3.14 (m, 2H), 2.42 (t, J=7.2 Hz, 2H), 2.30-2.17 (m, 1H), 1.93-1.84 (m, 2H), 1.49-1.41 (m, 4H), 1.37 (s, 9H), 1.36-1.28 (m, 4H).

### 4-[2-(7-Azaspiro[3.5]nonan-2-yl)ethylamino]-2-(2,6-dioxo-3-piperidyl)isoindoline-1,3-dione (Intermediate ATC)

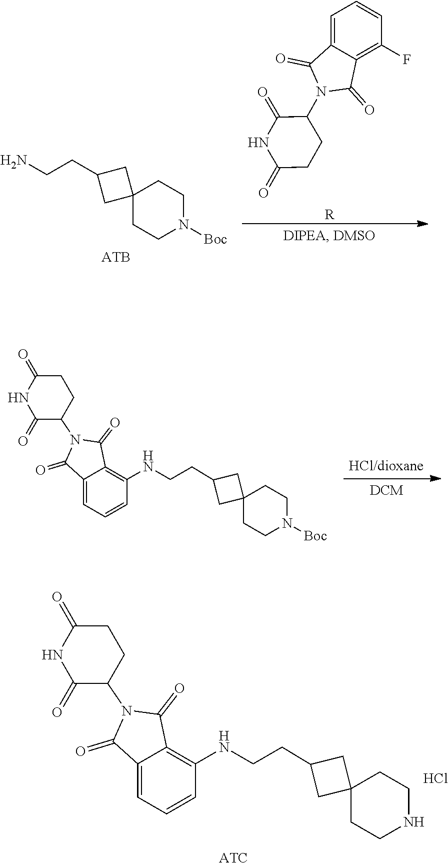

### Step 1—Tert-butyl 2-[2-[[2-(2,6-dioxo-3-piperidyl)-1,3-dioxo-isoindolin-4-yl]amino]ethyl]-7-azaspiro [3.5]nonane-7-carboxylate

To a solution of tert-butyl 2-(2-aminoethyl)-7-azaspiro[3.5]nonane-7-carboxylate (160 mg, 596 umol, Intermediate ATB) and 2-(2,6-dioxo-3-piperidyl)-4-fluoro-isoindoline-1,3-dione (181 mg, 655 umol, Intermediate R) in DMSO (3 mL) was added DIPEA (154 mg, 1.19 mmol). The reaction mixture was stirred at 130° C. for 2 hrs. On completion, the reaction mixture was diluted with water (10 mL) and extracted with EA (3×30 mL). The combined organic layers were washed with brine (20 mL), dried over Na₂SO₄, filtered and concentrated in vacuo. The residue was purified by reverse phase (0.1% FA) to give the title compound (200 mg, 63% yield) as yellow solid. ¹H NMR (400 MHz, DMSO-d₆) δ 11.08 (s, 1H), 7.58 (dd, J=7.2, 8.4 Hz, 1H), 7.07 (d, J=8.4 Hz, 1H), 7.02 (d, J=6.8 Hz, 1H), 6.47 (t, J=5.6 Hz, 1H), 5.04 (dd, J=5.2, 12.8 Hz, 1H), 3.28-3.24 (m, 2H), 3.24-3.20 (m, 2H), 3.19-3.14 (m, 2H), 2.93-2.80 (m, 1H), 2.63-2.54 (m, 2H), 2.31-2.22 (m, 1H), 2.07-1.99 (m, 1H), 1.98-1.90 (m, 2H), 1.74-1.64 (m, 2H), 1.51-1.38 (m, 6H), 1.37 (s, 9H).

### Step 2—4-[2-(7-Azaspiro[3.5]nonan-2-yl)ethylamino]-2-(2,6-dioxo-3-piperidyl)isoindoline-1,3-dione

To a solution of tert-butyl 2-[2-[[2-(2,6-dioxo-3-piperidyl)-1,3-dioxo-isoindolin-4-yl]amino]ethyl]-7-azaspiro[3.5]nonane-7-carboxylate (50.0 mg, 95.3 umol) in DCM (1 mL) was added HCl/dioxane (4 M, 0.5 mL). The reaction mixture was stirred at 20° C. for 1 hr. On completion, the reaction mixture was concentrated in vacuo to give the title compound (43.0 mg, 97% yield, HCl salt) as yellow solid. LC-MS (ESI⁺) m/z 425.3 (M+H)⁺.

### 4-(7-Azaspiro[3.5]nonan-2-ylamino)-2-(2,6-dioxo-3-piperidyl)isoindoline-1,3-dione (Intermediate AML)

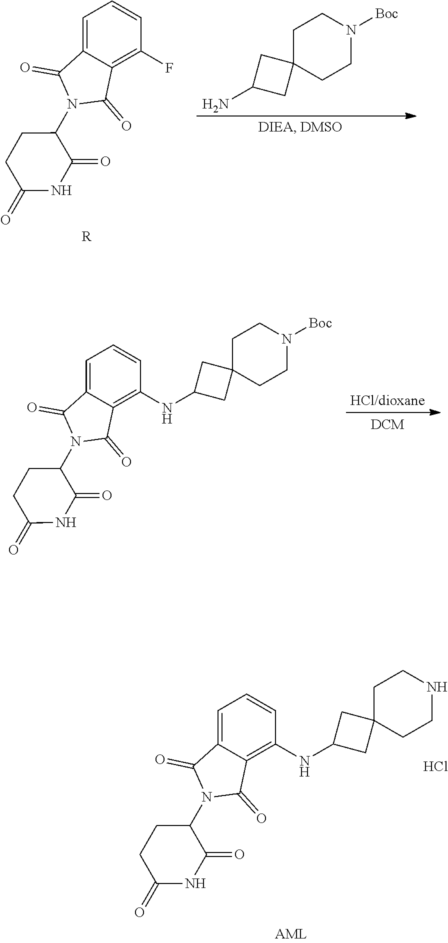

### Step 1—Tert-butyl 2-[[2-(2,6-dioxo-3-piperidyl)-1,3-dioxo-isoindolin-4-yl]amino]-7-azaspiro[3.5]nonane-7-carboxylate

To a mixture of 2-(2,6-dioxo-3-piperidyl)-4-fluoro-isoindoline-1,3-dione (0.50 g, 1.81 mmol, Intermediate R) and tert-butyl 2-amino-7-azaspiro[3.5]nonane-7-carboxylate (478 mg, 1.99 mmol, CAS #1239319-82-4) in DMSO (10 mL) was added DIPEA (468 mg, 3.62 mmol). The mixture was stirred at 130° C. for 2 hours. On completion, the reaction mixture was poured into the water (30 mL), and extracted with EA (2×20 mL). The combined organic phase was washed with brine (2×30 mL), dried with anhydrous Na₂SO₄, filtered and concentrated in vacuo to give the title compound (0.80 g, 89% yield) as a yellow solid. ¹H NMR (400 MHz, CDCl₃) δ 8.33 (s, 1H), 7.48 (d, J=7.8 Hz, 1H), 7.11 (d, J=7.2 Hz, 1H), 6.74 (d, J=8.4 Hz, 1H), 6.31 (d, J=5.6 Hz, 1H), 4.95-4.89 (m, 1H), 4.08-3.99 (m, 1H), 3.42-3.35 (m, 2H), 3.33-3.26 (m, 2H), 2.93-2.84 (m, 1H), 2.83-2.71 (m, 2H), 2.48-2.35 (m, 2H), 2.17-2.09 (m, 1H), 1.78-1.71 (m, 2H), 1.65-1.60 (m, 2H), 1.58-1.52 (m, 2H), 1.45 (s, 9H).

### Step 2—4-(7-Azaspiro[3.5]nonan-2-ylamino)-2-(2,6-dioxo-3-piperidyl)isoindoline-1,3-dione

To a mixture of tert-butyl 2-[[2-(2,6-dioxo-3-piperidyl)-1,3-dioxo-isoindolin-4-yl]amino]-7-azaspiro[3.5]nonane-7-carboxylate (0.80 g, 1.61 mmol) in DCM (20 mL) was added HCl/dioxane (4 M, 1.21 mL). The reaction mixture was stirred at 20° C. for 0.5 hour. On completion, the reaction mixture was concentrated in vacuo to give the title compound (690 mg, 98% yield) as a yellow solid. LC-MS (ESI⁺) m/z 397.2 (M+H)⁺.

### 3-[3-Methyl-4-[4-(methylamino)-1-piperidyl]-2-oxo-benzimidazol-1-yl]piperidine-2,6-dione (Intermediate AQK)

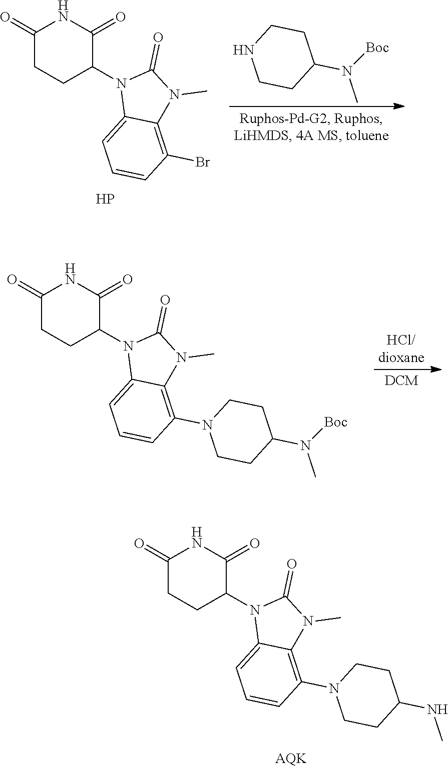

### Step 1—Tert-butyl N-[1-[1-(2,6-dioxo-3-piperidyl)-3-methyl-2-oxo-benzimidazol-4-yl]-4-piperidyl]-N-methyl-carbamate

To a solution of 3-(4-bromo-3-methyl-2-oxo-benzimidazol-1-yl)piperidine-2,6-dione (500 mg, 1.48 mmol, Intermediate HP), tert-butyl N methyl-N-(4-piperidyl)carbamate (633 mg, 2.96 mmol, CAS #108612-54-0) and 4A molecular sieves (500 mg) in toluene (10 mL) was added [2-(2-aminophenyl)phenyl]-chloro-palladium; dicyclohexyl-[2-(2,6-diisopropoxyphenyl)phenyl]phosphane (229 mg, 295 umol), RuPhos (138 mg, 295 umol) and LiHMDS (1.00 M, 8.87 mL) under N₂ atmosphere. The mixture was stirred at 80° C. for 1 hr under N₂ atmosphere. On completion, the mixture was concentrated in vacuo. The residue was diluted with DMF (6 mL), filtered and the filtrate was acidified with FA until the pH=5. The filtrate was concentrated in vacuo. The residue was purified by reverse phase (0.1% FA) and silica gel column (PE:EA=1:1) to give the title compound (70 mg, 10% yield) as a yellow solid. ¹H NMR (400 MHz, DMSO-d₆) δ 11.08 (s, 1H), 7.01-6.84 (m, 3H), 5.42-5.27 (m, 1H), 3.64 (s, 3H), 3.36-3.33 (m, 1H), 3.22-3.09 (m, 2H), 2.94-2.77 (m, 3H), 2.75 (s, 3H), 2.70-2.57 (m, 2H), 2.04-1.98 (m, 1H), 1.95-1.84 (m, 2H), 1.72-1.59 (m, 2H), 1.42 (s, 9H), LC-MS (ESI⁺) m/z 472.2 (M+H)⁺.

### Step 2—3-[3-Methyl-4-[4-(methylamino)-1-piperidyl]-2-oxo-benzimidazol-1-yl]piperidine-2,6-dione

To a solution of tert-butyl N-[1-[1-(2,6-dioxo-3-piperidyl)-3-methyl-2-oxo-benzimidazol-4-yl]-4-piperidyl]-N-methylcarbamate (70.0 mg, 148 umol) in DCM (3.00 mL) was added HCl/dioxane (4.00 M, 2.00 mL). The mixture was stirred at 25° C. for 0.5 hr. On completion, the mixture was concentrated in vacuo to give the title compound (60 mg, 99% yield, HCl salt) as a yellow solid. LC-MS (ESI⁺) m/z 372.3 (M+H)⁺.

### (4-Aminocyclohexyl)methanol (Intermediate ATD)

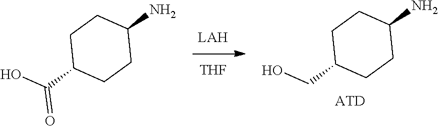

To a solution of LAH (26.5 g, 698 mmol) in THF (900 mL) was added 4-aminocyclohexanecarboxylic acid (50.0 g, 349 mmol, CAS #3685-25-4) dropwise at 0° C. The reaction mixture was stirred at 70° C. for 16 hrs. On completion, the reaction mixture was quenched by water (28 mL), then 10% NaOH aqueous (80 mL) and filtered. The filter cake was washed with DCM/THF=½ (5×800 mL). The combined organic layers were concentrated in vacuo to give the title compound (40.0 g, 88% yield) as light yellow solid. ¹H NMR (400 MHz, DMSO-d₆) δ 4.33 (br s, 1H), 3.18 (d, J=6.4 Hz, 2H), 2.41 (tt, J=4.0, 10.4 Hz, 1H), 1.80-1.59 (m, 4H), 1.29-1.18 (m, 1H), 1.02-0.76 (m, 4H).

### [4-(5-Amino-6-methoxy-indazol-2-yl)cyclohexyl]methanol (Intermediate ATE)

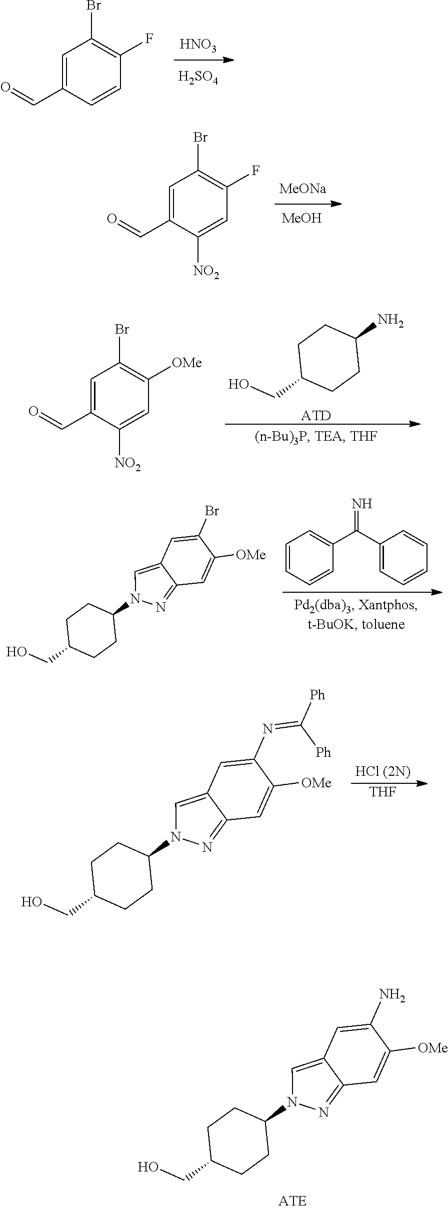

### Step 1—5-Bromo-4-fluoro-2-nitro-benzaldehyde

To a solution of 3-bromo-4-fluoro-benzaldehyde (10.0 g, 49.2 mmol, CAS #77771-02-9) in H₂SO₄ (80 mL) was added HNO₃ (9.55 g, 98.5 mmol, 65% solution) dropwise at 0° C. The reaction mixture was stirred at 0-20° C. for 12 hrs. On completion, the reaction mixture was poured into cold water (600 mL) and extracted with EA (3×200 mL). The combined organic layers were washed with brine (200 mL), dried over Na₂SO₄, filtered and concentrated in vacuo. The residue was purified by silica gel chromatography (PE:EA=200:1) to give the title compound (9.60 g, 78% yield) as yellow solid. ¹H NMR (400 MHz, DMSO-d₆) δ 10.14 (s, 1H), 8.31 (d, J=8.0 Hz, 1H), 8.26 (d, J=6.8 Hz, 1H).

### Step 2—5-Bromo-4-methoxy-2-nitro-benzaldehyde

To a solution of 5-bromo-4-fluoro-2-nitro-benzaldehyde (4.00 g, 16.1 mmol) in MeOH (40 mL) was added NaOMe (1.31 g, 24.1 mmol) at 0° C. The reaction mixture was stirred at 0-20° C. for 16 hrs. On completion, the reaction mixture was quenched by water (10 mL), diluted with water (60 mL) and filtered. The filter cake was dried in vacuo to give the title compound (2.10 g, 40% yield) as yellow solid. ¹H NMR (400 MHz, DMSO-d₆) δ 10.04 (s, 1H), 8.16 (s, 1H), 7.79 (s, 1H), 4.06 (s, 3H).

### Step 3—[4-(5-Bromo-6-methoxy-indazol-2-yl)cyclohexyl]methanol

A mixture of 5-bromo-4-methoxy-2-nitro-benzaldehyde (1.90 g, 7.31 mmol) and (4-aminocyclohexyl) methanol (1.04 g, 8.04 mmol, Intermediate ATD) in IPA (20 mL) was stirred at 80° C. for 3 hrs. Then the solution was cooled to 25° C., and tributylphosphane (4.43 g, 21.9 mmol) was added and the reaction mixture was stirred at 80° C. for 16 hrs. On completion, the reaction mixture was concentrated in vacuo. The residue was purified by silica gel chromatography (PE:EA=1:1) to give the impure product. The impure product was triturated with PE (30 mL) to give the title compound (1.50 g, 60% yield) as white solid. ¹H NMR (400 MHz, DMSO-d₆) δ 8.27 (s, 1H), 7.95 (s, 1H), 7.10 (s, 1H), 4.47 (t, J=5.6 Hz, 1H), 4.42-4.31 (m, 1H), 3.86 (s, 3H), 3.28 (t, J=6.0 Hz, 2H), 2.17-2.04 (m, 2H), 1.95-1.79 (m, 4H), 1.54-1.39 (m, 1H), 1.21-1.05 (m, 2H).

### Step 4—[4-[5-(Benzhydrylideneamino)-6-methoxy-indazol-2-yl]cyclohexyl]methanol

The reaction was performed in parallel two batches: A mixture of [4-(5-bromo-6-methoxy-indazol-2-yl)cyclohexyl]methanol (500 mg, 1.47 mmol), diphenylmethanimine (534 mg, 2.95 mmol), Pd₂(dba)₃ (134 mg, 147 umol), Xantphos (170 mg, 294 umol) and t-BuOK (496 mg, 4.42 mmol) in dioxane (10 mL) was stirred at 80° C. for 1 hr under nitrogen. On completion, the combined reaction mixture was quenched by methanol (1 mL), filtered and the filtrate was concentrated in vacuo. The residue was purified by silica gel chromatography (PE:EA=1:2) to give the title compound (600 mg, 11% yield) as yellow solid. LC-MS (ESI⁺) m/z 440.2 (M+H)⁺.

### Step 5—[4-(5-Amino-6-methoxy-indazol-2-yl)cyclohexyl]methanol

To a solution of [4-[5-(benzhydrylideneamino)-6-methoxy-indazol-2-yl]cyclohexyl]methanol (650 mg, 1.48 mmol) in THF (3 mL) was added HCl/dioxane (2 M in water, 18.7 mL). The reaction mixture was stirred at 20° C. for 1 hr. On completion, the reaction mixture was concentrated in vacuo. The residue was purified by reverse phase (0.1% NH₃·H₂O) to give the title compound (180 mg, 33% yield) as yellow solid. LC-MS (ESI⁺) m/z 276.1 (M+H)⁺.

### 6-(1,1-Difluoroethyl)-N-[2-(4-formylcyclohexyl)-6-methoxy-indazol-5-yl]pyridine-2-carboxamide (Intermediate ATF)

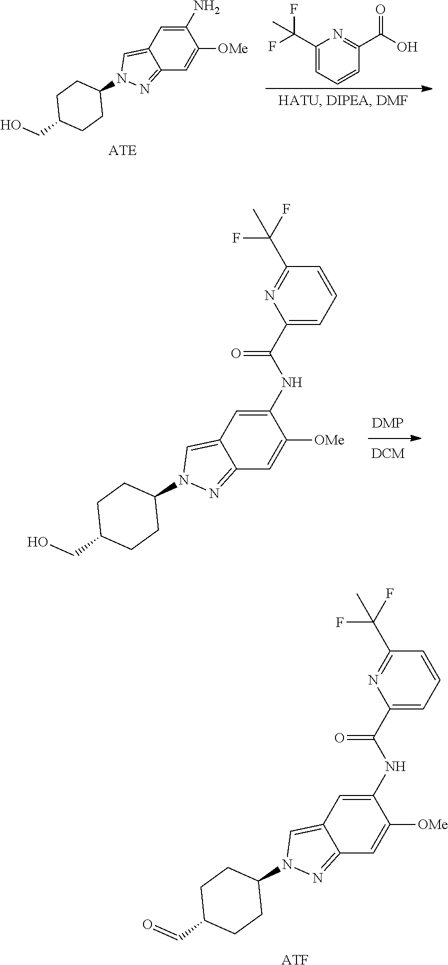

### Step 1—6-(1,1-Difluoroethyl)-N-[2-[4-(hydroxymethyl)cyclohexyl]-6-methoxy-indazol-5-yl]pyridine-2-carboxamide

To a solution of 6-(1,1-difluoroethyl)pyridine-2-carboxylic acid (80.8 mg, 432 umol, CAS #1211529-86-0), HATU (305 mg, 802 umol) and DIPEA (239 mg, 1.85 mmol) in DMF (3 mL) was added [4-(5-amino-6-methoxy-indazol-2-yl)cyclohexyl]methanol (170 mg, 617 umol, Intermediate ATE). The reaction mixture was stirred at 25° C. for 1 hr. On completion, the reaction mixture was quenched by water (0.1 mL) and concentrated in vacuo. The residue was purified by reverse phase (0.1% FA) to give the title compound (110 mg, 40% yield) as yellow solid. ¹H NMR (400 MHz, DMSO-d₆) δ 10.67 (s, 1H), 8.67 (s, 1H), 8.40-8.26 (m, 3H), 8.01 (dd, J=2.0, 6.8 Hz, 1H), 7.16 (s, 1H), 4.49 (t, J=5.2 Hz, 1H), 4.41-4.29 (m, 1H), 3.99 (s, 3H), 3.31-3.27 (m, 2H), 2.23-2.10 (m, 3H), 1.97-1.80 (m, 4H), 1.56-1.39 (m, 1H), 1.31-1.03 (m, 4H).

### Step 2—6-(1,1-Difluoroethyl)-N-[2-(4-formylcyclohexyl)-6-methoxy-indazol-5-yl]pyridine-2-carboxamide

To a solution of 6-(1,1-difluoroethyl)-N-[2-[4-(hydroxymethyl)cyclohexyl]-6-methoxy-indazol-5-yl]pyridine-2-carboxamide (100 mg, 224 umol) in DCM (1 mL) was added DMP (114 mg, 269 umol). The reaction mixture was stirred at 20° C. for 1 hr. On completion, the reaction mixture was quenched by saturated Na₂S₂SO₃ (1 mL), basified with saturated NaHCO₃ till pH=7-8 and extracted with DCM (3×20 mL). The combined organic layers were washed with brine (2×15 mL), dried over Na₂SO₄, filtered and concentrated in vacuo to give the title compound (90.0 mg, 90% yield) as yellow solid. ¹H NMR (400 MHz, DMSO-d₆) δ 10.67 (s, 1H), 9.64 (s, 1H), 8.67 (s, 1H), 8.35-8.25 (m, 3H), 8.01 (dd, J=2.4, 6.8 Hz, 1H), 7.16 (s, 1H), 4.47-4.32 (m, 1H), 3.99 (s, 3H), 2.46-2.37 (m, 1H), 2.23-2.13 (m, 3H), 2.12-2.06 (m, 2H), 2.02-1.89 (m, 2H), 1.51-1.37 (m, 2H), 1.29-1.21 (m, 2H).

### 6-(Trifluoromethyl)pyridine-2-carboxamide (Intermediate ATI)

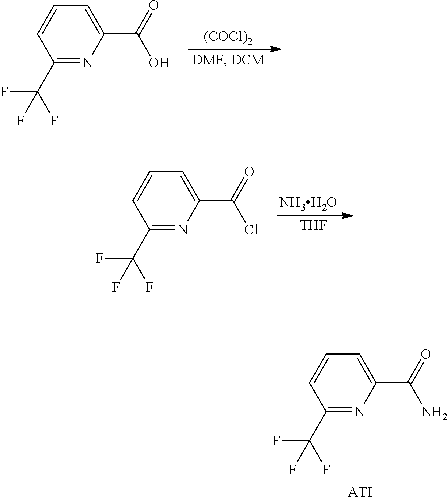

### Step 1—6-(Trifluoromethyl)pyridine-2-carbonyl chloride

To a mixture of 6-(trifluoromethyl)pyridine-2-carboxylic acid (21.0 g, 109 mmol, CAS #131747-42-7) and DMF (401 mg, 5.49 mmol) in DCM (300 mL) was added (COCl)₂ (27.9 g, 219 mmol) at 0° C. The mixture was stirred at 25° C. for 1 hour. On completion, the reaction mixture was concentrated in vacuo to give the title compound (22 g, 950% yield) as light yellow oil.

### Step 2—6-(Trifluoromethyl)pyridine-2-carboxamide

A solution of 6-(trifluoromethyl)pyridine-2-carbonyl chloride (21.5 g, 102 mmol) in THF (100 mL) was added NH₃·H₂O (143 g, 1.03 mol, 158 mL, 25% solution) at 0° C. The mixture was stirred at 25° C. for 1 hour. On completion, the reaction mixture was concentrated in vacuo to remove THF and then filtered to give the filter cake as title product (19 g, 90% yield) as light yellow solid. ¹H NMR (400 MHz, DMSO-d₆) δ 8.35-8.24 (m, 2H), 8.08 (dd, J=1.6, 6.8 Hz, 1H), 8.05-7.78 (m, 2H); LC-MS (ESI⁺) m/z 191.0 (M+H)⁺.

### N-[2-(4-formylcyclohexyl)-6-methoxy-indazol-5-yl]-6-(trifluoromethyl)pyridine-2-carboxamide (Intermediate ATJ)

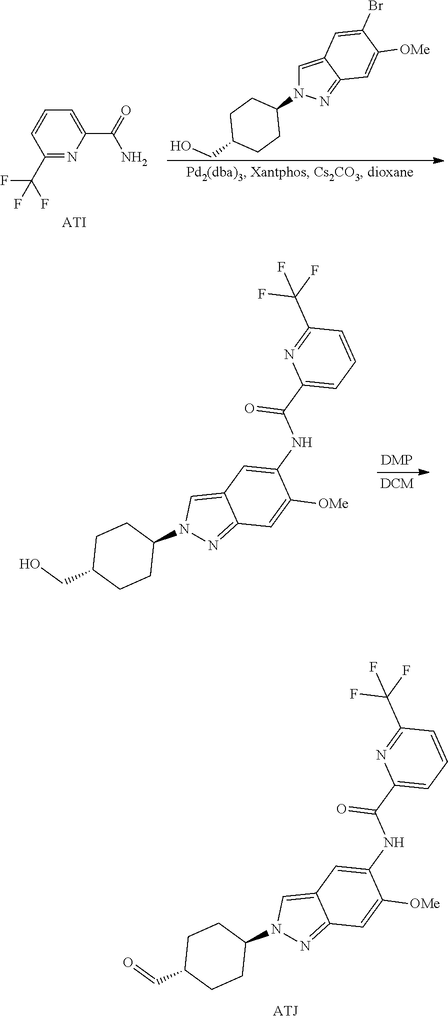

### Step 1—N-[2-[4-(hydroxymethyl)cyclohexyl]-6-methoxy-indazol-5-yl]-6-(trifluoromethyl)pyridine-2-carboxamide

To a solution of [4-(5-bromo-6-methoxy-indazol-2-yl)cyclohexyl]methanol (6.50 g, 19.1 mmol, synthesized via Steps 1-3 of Intermediate ATE) in dioxane (150 mL) was added Pd₂(dba)₃ (1.75 g, 1.92 mmol), Xantphos (2.22 g, 3.83 mmol), Cs₂CO₃ (12.4 g, 38.3 mmol) and 6-(trifluoromethyl)pyridine-2-carboxamide (4.01 g, 21.0 mmol, Intermediate ATI). The mixture was stirred at 80° C. for 16 hours. On completion, the reaction was filtered and concentrated in vacuo to give a residue. The residue was diluted with DCM (150 mL), and washed with water (2×30 mL). The combined organic layers was dried over Na₂SO₄, filtered and concentrated in vacuo to give a residue. The residue was purified by column chromatography (SiO₂, PE/EA=1/1) to give the title compound (6.50 g, 75% yield) as gray solid. ¹H NMR (400 MHz, DMSO-d₆) δ 10.48 (s, 1H), 8.67 (s, 1H), 8.50-8.41 (m, 1H), 8.41-8.33 (m, 1H), 8.31 (s, 1H), 8.19 (dd, J=0.8, 7.6 Hz, 1H), 7.14 (s, 1H), 4.77-4.26 (m, 2H), 4.04-3.92 (m, 1H), 3.97 (s, 2H), 3.29 (d, J=6.0 Hz, 2H), 2.22-2.06 (m, 2H), 1.96-1.79 (m, 4H), 1.55-1.40 (m, 1H), 1.25-1.03 (m, 2H); LC-MS (ESI⁺) m/z 449.4 (M+H)⁺.

### Step 2—N-[2-(4-formylcyclohexyl)-6-methoxy-indazol-5-yl]-6-(trifluoromethyl)pyridine-2-carboxamide

To a solution of N-[2-[4-(hydroxymethyl)cyclohexyl]-6-methoxy-indazol-5-yl]-6-(trifluoromethyl) pyridine-2-carboxamide (6.70 g, 14.9 mmol) in DCM (200 mL) was added DMP (7.60 g, 17.9 mmol). The mixture was stirred at 25° C. for 2 hours. On completion, the reaction mixture was diluted with DCM (100 mL) and quenched by saturated Na₂S₂O₃ (100 mL) and saturated NaHCO₃ (100 mL) at 0° C. The mixture was then stirred at 25° C. for 30 minutes. After, the organic layers was separated, then washed with saturated NaHCO₃ (100 mL) and saturated NaCl (100 mL). The organic layers dried over Na₂SO₄, filtered and concentrated in vacuo to give a residue. The residue was triturated with (EA/DCM=10/1) to give the title compound (6.6 g, 95% yield) as light yellow solid. ¹H NMR (400 MHz, DMSO-d₆) δ 10.49 (s, 1H), 9.64 (s, 1H), 8.68 (s, 1H), 8.45 (d, J=8.0 Hz, 1H), 8.38 (t, J=8.0 Hz, 1H), 8.31 (s, 1H), 8.19 (d, J=7.6 Hz, 1H), 7.14 (s, 1H), 4.42-4.34 (m, 1H), 3.97 (s, 3H), 2.46-2.36 (m, 1H), 2.20 (dd, J=2.8, 12.4 Hz, 2H), 2.10 (d, J=11.6 Hz, 2H), 1.99-1.89 (m, 2H), 1.48-1.38 (m, 2H); LC-MS (ESI⁺) m/z 447.2 (M+H)⁺.

### ((1R,4r)-4-(5-bromo-6-fluoro-2H-indazol-2-yl)cyclohexyl)methanol (Intermediate BKP)

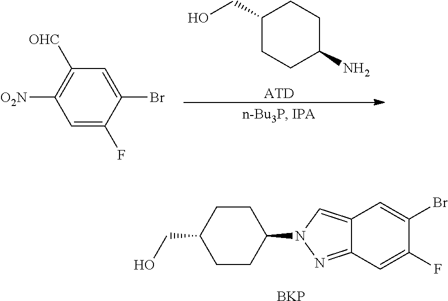

A mixture of 5-bromo-4-fluoro-2-nitro-benzaldehyde (500 mg, 2.02 mmol, synthesized via Step 1 of Intermediate ATE) and ((1r,4r)-4-aminocyclohexyl)methanol (286 mg, 2.22 mmol, Intermediate ATD) in IPA (6.00 mL) was stirred at 80° C. for 4 hrs under N₂. It was cooled to 25° C., then tributylphosphine (1.22 g, 6.05 mmol, 1.49 mL) was added. Then the reaction mixture was stirred at 80° C. for 16 hrs. On completion, the reaction mixture was concentrated in vacuo to give a residue. The residue was diluted with H₂O (10 mL) and extracted with EA (3×10 mL). The combined organic layers were washed with brine (2×20 mL), dried over anhydrous Na₂SO₄ then filtered. The filtrate was concentrated in vacuo to give a residue. The residue was purified by column chromatography (SiO2, petroleum ether/ethyl acetate=1/1) to give the title compound (500 mg, 75% yield) as a yellow solid. ¹H NMR (400 MHz, CDCl₃) δ 7.91 (s, 1H), 7.87 (d, J=6.8 Hz, 1H), 7.39 (d, J=9.6 Hz, 1H), 4.40-432 (m, 1H), 3.55 (d, J=6.4 Hz, 2H), 2.37-2.28 (m, 2H), 2.11-2.02 (m, 2H), 2.00-1.89 (m, 2H), 1.46-1.20 (m, 4H). LC-MS (ESI⁺) m/z 327.0 (M+H)⁺.

### N-(6-fluoro-2-((1r,4r)-4-formylcyclohexyl)-2H-indazol-5-yl)-6-(trifluoromethyl) picolinamide (Intermediate BKQ)

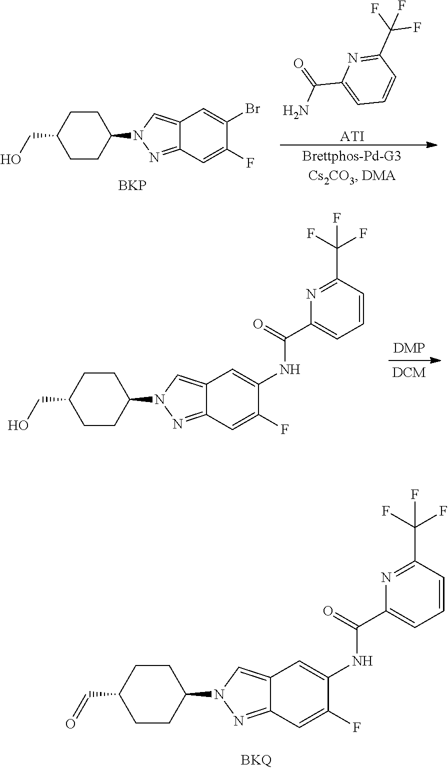

### Step 1—N-(6-fluoro-2-((1r,4r)-4-(hydroxymethyl)cyclohexyl)-2H-indazol-5-yl)-6-(trifluoromethyl) picolinamide

A mixture of ((1r,4r)-4-(5-bromo-6-fluoro-2H-indazol-2-yl)cyclohexyl)methanol (400 mg, 1.22 mmol, Intermediate BKP), 6-(trifluoromethyl)pyridine-2-carboxamide (255 mg, 1.34 mmol, Intermediate ATI), BrettPhos Pd G3 (55.4 mg, 61.1 umol) and Cs₂CO₃ (1.19 g, 3.67 mmol) in DMA (6 mL) was degassed and purged with N₂ for 3 times. The mixture was stirred at 100° C. for 2 hrs under N₂ atmosphere. On completion, the reaction mixture was filtered and the filtrate was concentrated in vacuo to give a residue. The residue was diluted with H₂O (50 mL) and extracted with EA (2×50 mL). The combined organic layers were washed with brine (2×60 mL), dried over anhydrous Na₂SO₄, filtered and concentrated in vacuo to give a residue. The residue was purified by reverse-phase (0.1% FA condition) to give the crude product. The crude product was triturated with PE (5 mL) at 25° C. for 0.5 hr to give the title compound (190 mg, 35% yield) as a yellow solid. ¹H NMR (400 MHz, CDCl₃) δ 10.24 (d, J=2.8 Hz, 1H), 8.82 (d, J=7.6 Hz, 1H), 8.51 (d, J=7.6 Hz, 1H), 8.15 (t, J=8.0 Hz, 1H), 7.97 (s, 1H), 7.90 (d, J=7.6 Hz, 1H), 7.44 (d, J=11.6 Hz, 1H), 4.42-4.34 (m, 1H), 3.57 (d, J=6.0 Hz, 2H), 2.42-2.30 (m, 2H), 2.12-1.93 (m, 4H), 1.72-1.65 (m, 1H), 1.33-1.21 (m, 2H). LC-MS (ESI⁺) m/z 437.2 (M+H)⁺.

### Step 2—N-(6-fluoro-2-((1r,4r)-4-formylcyclohexyl)-2H-indazol-5-yl)-6-(trifluoromethyl) picolinamide

To a solution of N-(6-fluoro-2-((1r,4r)-4-(hydroxymethyl)cyclohexyl)-2H-indazol-5-yl)-6-(trifluoromethyl)picolinamide (160 mg, 366 umol) in DCM (3.00 mL) was added DMP (202 mg, 476 umol) at 0° C. The reaction mixture was stirred at 20° C. for 1 hr. On completion, the reaction mixture was quenched by addition saturated solution of Na₂S₂O₃ (5 mL), and saturated solution of NaHCO₃ (4 mL), then extracted with DCM (3×10 mL). The combined organic layers were washed with brine (2×20 mL). The organic was dried over anhydrous Na₂SO₄, filtered and concentrated in vacuo to give the title compound (140 mg, 88% yield) as a yellow solid. ¹H NMR (400 MHz, CDCl₃) δ 10.24 (d, J=3.2 Hz, 1H), 9.73 (s, 1H), 8.82 (d, J=7.8 Hz, 1H), 8.51 (d, J=7.6 Hz, 1H), 8.15 (t, J=8.0 Hz, 1H), 7.97 (s, 1H), 7.92-7.87 (m, 1H), 7.44 (d, J=11.6 Hz, 1H), 4.42-4.34 (m, 1H), 2.47-2.37 (m, 3H), 2.32-2.26 (m, 2H), 2.12-2.01 (m, 2H), 1.60-1.50 (m, 2H). LC-MS (ESI⁺) m/z 435.1 (M+H)⁺.

### (1R,3R)-methyl 3-(6-fluoro-5-(6-(trifluoromethyl)picolinamido)-2H-indazol-2-yl) cyclobutanecarboxylate (Intermediate BQD) and (1S,3S)-methyl 3-(6-fluoro-5-(6-(trifluoromethyl)picolinamido)-2H-indazol-2-yl)cyclobutanecarboxylate (Intermediate BPH)

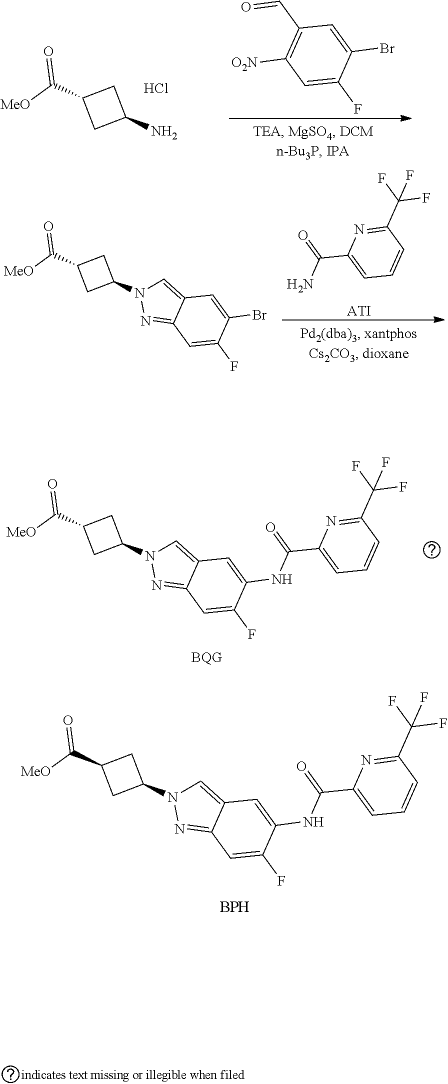

### Step 1—(1R,3R)-methyl 3-(5-bromo-6-fluoro-2H-indazol-2-yl)cyclobutanecarboxylate

To a solution of methyl 3-aminocyclobutanecarboxylate (1.00 g, 6.04 mmol, HCl salt, CAS #74316-29-3) and 5-bromo-4-fluoro-2-nitro-benzaldehyde (1.25 g, 5.03 mmol, synthesized via Step 1 of Intermediate ATE) in DCM (10 mL) was added TEA (610 mg, 6.04 mmol) and MgSO₄ (908 mg, 7.55 mmol) at 0° C. and the resulting mixture was stirred at rt for 2 hours. Then IPA (10 mL) and tributylphosphane (3.05 g, 15.1 mmol) were added and the mixture was stirred at 80° C. for 10 hours. The reaction mixture was concentrated under reduced pressure to give a residue. The residue was purified by column chromatography (SiO₂, Petroleum ether/Ethyl acetate=0/1 to 5/1) to give the title compound (1.30 g, 66% yield) as a white solid. LC-MS (ESI⁺) m/z 329.0 (M+H)⁺. ¹H NMR (400 MHz, CDCl₃) δ 7.91 (s, 1H), 7.88 (d, J=6.8 Hz, 1H), 7.43 (d, J=9.4 Hz, 1H), 5.36-5.20 (m, 1H), 3.78 (s, 3H), 3.41-3.25 (m, 1H), 3.15-3.03 (m, 2H), 2.92-2.80 (m, 2H).

### Step 2—(1R,3R)-methyl 3-(6-fluoro-5-(6-(trifluoromethyl)picolinamido)-2H-indazol-2-yl) cyclobutanecarboxylate and (1S,3S)-methyl 3-(6-fluoro-5-(6-(trifluoromethyl)picolinamido)-2H-indazol-2-yl)cyclobutanecarboxylate

To a solution of methyl 3-(5-bromo-6-fluoro-indazol-2-yl)cyclobutanecarboxylate (500 mg, 1.53 mmol) and 6-(trifluoromethyl)pyridine-2-carboxamide (319 mg, 1.68 mmol, Intermediate ATI) in DMA (5 mL) was added Cs₂CO₃ (995 mg, 3.06 mmol) and BrettPhos-Pd-G3 (138 mg, 152 umol) and the mixture was stirred at 90° C. for 12 hours. The reaction mixture was partitioned between H₂O (50 mL) and EA (100 mL). The organic phase was separated, washed with brine (50 mL), dried over Na₂SO₄, filtered and concentrated under reduced pressure to give a residue. The residue was purified by column chromatography (SiO₂, Petroleum ether/Ethyl acetate=10/1 to 3/1) to give compound (1R,3R)-methyl 3-(6-fluoro-5-(6-(trifluoromethyl)picolinamido)-2H-indazol-2-yl) cyclobutanecarboxylate (176 mg, 21% yield) as an off-white solid (¹H NMR (400 MHz, CDCl₃) δ 10.25 (d, J=3.2 Hz, 1H), 8.82 (d, J=7.6 Hz, 1H), 8.51 (d, J=7.8 Hz, 1H), 8.15 (t, J=7.8 Hz, 1H), 7.96 (d, J=0.8 Hz, 1H), 7.90 (dd, J=0.8, 7.8 Hz, 1H), 7.47 (d, J=11.6 Hz, 1H), 5.30 (t, J=8.2 Hz, 1H), 3.79 (s, 3H), 3.40-3.29 (m, 1H), 3.16-3.05 (m, 2H), 2.88 (m, 2H); LC-MS (ESI⁺) m/z 437.1 (M+H)⁺) and (1S,3S)-methyl 3-(6-fluoro-5-(6-(trifluoromethyl)picolinamido)-2H-indazol-2-yl)cyclobutanecarboxylate (240 mg, 35% yield) as an off-white solid (¹H NMR (400 MHz, CDCl₃) δ 10.26 (d, J=2.4 Hz, 1H), 8.84 (d, J=7.6 Hz, 1H), 8.51 (d, J=7.6 Hz, 1H), 8.15 (t, J=8.0 Hz, 1H), 8.06 (s, 1H), 7.90 (dd, J=0.9, 7.8 Hz, 1H), 7.46 (d, J=11.6 Hz, 1H), 5.02 (m, 1H), 3.76 (s, 3H), 3.15-3.03 (m, 1H), 3.02-2.84 (m, 4H); LC-MS (ESI⁺) m/z 437.1 (M+H)⁺).

### N-(6-fluoro-2-((1S,3S)-3-formylcyclobutyl)-2H-indazol-5-yl)-6-(trifluoromethyl)picolinamide (Intermediate BPI)

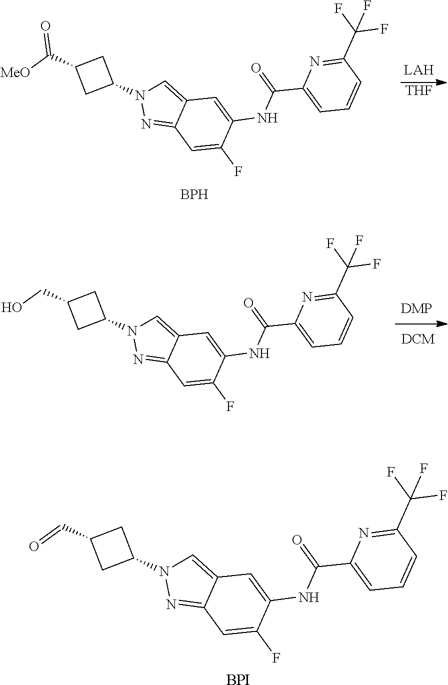

### Step 1—N-(6-fluoro-2-((1s,3s)-3-(hydroxymethyl)cyclobutyl)-2H-indazol-5-yl)-6-(trifluoromethyl) picolinamide

To a solution of methyl 3-[6-fluoro-5-[[6-(trifluoromethyl)pyridine-2-carbonyl]amino]indazol-2-yl]cyclobutanecarboxylate (100 mg, 229 umol, Intermediate BPH) in THF (2 mL) was added LiAlH₄ (17.3 mg, 458 umol) and the mixture was stirred at 0° C. for 1 hour. On completion, the reaction mixture was quenched by addition of 150% seignette salt (0.1 mL) at 0° C., and then diluted with THF (10 mL). The mixture was then filtered and concentrated under reduced pressure to give the title compound (90.0 mg, 88% yield) as an off-white solid. ¹H NMR (400 MHz, CDCl₃) δ 10.25 (d, J=3.0 Hz, 1H), 8.81 (d, J=7.8 Hz, 1H), 8.51 (d, J=7.8 Hz, 1H), 8.15 (t, J=7.8 Hz, 1H), 7.99 (d, J=0.6 Hz, 1H), 7.90 (dd, J=0.8, 7.8 Hz, 1H), 7.45 (d, J=11.8 Hz, 1H), 4.99-4.85 (m, 1H), 3.86-3.69 (m, 3H), 2.84-2.69 (m, 2H), 2.64-2.42 (m, 3H); LC-MS (ESI⁺) m/z 409.0 (M+H)⁺.

### Step 2—N-(6-fluoro-2-((1s,3s)-3-formylcyclobutyl)-2H-indazol-5-yl)-6-(trifluoromethyl)picolin amide

To a solution of N-[6-fluoro-2-[3-(hydroxymethyl)cyclobutyl]indazol-5-yl]-6-(trifluoromethyl) pyridine-2-carboxamide (90.0 mg, 220 umol) in DCM (1 mL) was added DMP (140 mg, 330 umol) and the mixture was stirred at 20° C. for 1 hour. The reaction mixture was quenched by addition of sat. NaHCO₃ (10 mL) and sat. Na₂SO₃ (10 mL) at 0° C., and then diluted with H₂O (10 mL) and extracted with EA (3×30 mL). The combined organic layers were washed with brine (30 mL), dried over Na₂SO₄, filtered and concentrated under reduced pressure to give the title compound (89.0 mg, 92% yield) as a brown solid. LC-MS (ESI⁺) m/z 407.0 (M+H)⁺.

### N-[6-fluoro-2-(2-iodoethyl)indazol-5-yl]-6-(trifluoromethyl)pyridine-2-carboxamide (Intermediate BPJ)

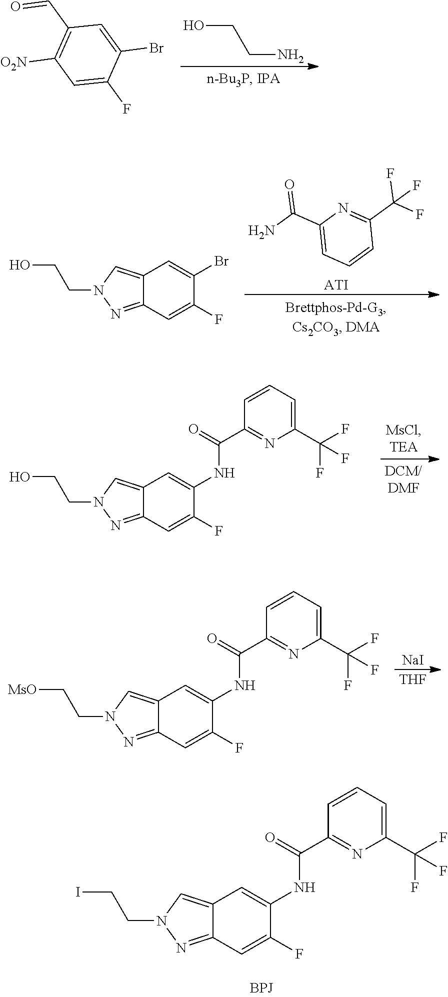

### Step 1—2-(5-Bromo-6-fluoro-indazol-2-yl)ethanol

A mixture of 5-bromo-4-fluoro-2-nitro-benzaldehyde (500 mg, 2.02 mmol) and 2-aminoethanol (135 mg, 2.22 mmol, 134 uL, CAS #141-43-5) in IPA (6.00 mL) was stirred at 80° C. for 4 hrs under N₂. The reaction was then cooled to 25° C. and tributylphosphane (1.23 g, 6.06 mmol, 1.50 mL) was added. The reaction mixture was stirred at 80° C. for 16 hrs. On completion, the reaction mixture was concentrated in vacuo to give a residue. The residue was diluted with H₂O (10 mL) and extracted with EA (3×10 mL). The combined organic layers were washed with brine (2×20 mL), dried over anhydrous Na₂SO₄, filtered and concentrated in vacuo to give a residue. The residue was purified by column chromatography (SiO₂, Petroleum ether/Ethyl acetate=3/1, Rf=0.12) to compound (250 mg, 48% yield) as a white solid. ¹H NMR (400 MHz, DMSO-d₆) δ 8.40 (s, 1H), 8.16 (d, J=7.2 Hz, 1H), 7.57 (d, J=10.4 Hz, 1H), 4.99 (t, J=5.2 Hz, 1H), 4.44 (t, J=5.2 Hz, 2H), 3.85 (q, J=5.2 Hz, 2H). LC-MS (ESI⁺) m/z 258.9 (M+H)⁺.

### Step 2—N-[6-fluoro-2-(2-hydroxyethyl)indazol-5-yl]-6-(trifluoromethyl)pyridine-2-carboxamide

A mixture of 2-(5-bromo-6-fluoro-indazol-2-yl)ethanol (100 mg, 386 umol), 6-(trifluoromethyl) pyridine-2-carboxamide (88.1 mg, 463.19 umol, Intermediate ATI), BrettPhos Pd G₃ (17.5 mg, 19.3 umol) and Cs₂CO₃ (251 mg, 772 umol) in DMA (2.00 mL) was degassed and purged with N₂ three times. The mixture was then stirred at 90° C. for 5 hrs under N₂ atmosphere. On completion, the reaction mixture was filtered and the filtrate was concentrated in vacuo to give a residue. The residue was diluted with H₂O (50 mL) and extracted with EA (2×50 mL). The combined organic layers were washed with brine (2×60 mL), dried over anhydrous Na₂SO₄, filtered and concentrated in vacuo to give a residue. The residue was purified by reverse phase (0.10% FA condition) to give the title compound (85.0 mg, 59% yield) as a yellow solid. ¹H NMR (400 MHz, DMSO-d₆) δ 10.24 (s, 1H), 8.48-8.33 (m, 4H), 8.21 (d, J=7.6 Hz, 1H), 7.53 (d, J=12.0 Hz, 1H), 5.08-4.95 (m, 1H), 4.44 (t, J=5.6 Hz, 2H), 3.95-3.85 (m, 2H); LC-MS (ESI⁺) m/z 368.8 (M+H)⁺.

### Step 3—2-[6-Fluoro-5-[[6-(trifluoromethyl)pyridine-2-carbonyl]amino]indazol-2-yl]ethyl methane sulfonate

To a solution of N-[6-fluoro-2-(2-hydroxyethyl)indazol-5-yl]-6-(trifluoromethyl)pyridine-2-carboxamide (80.0 mg, 217 umol) and TEA (65.9 mg, 651 umol, 90.7 uL) in DCM (5.00 mL) and DMF (1.00 mL) was added MsCl (49.7 mg, 434 umol, 33.6 uL) at 0° C., then the mixture was stirred at 25° C. for 1 hr. On completion, the reaction mixture was quenched by the addition of ice water (0.5 mL), and then diluted with water (10 mL) and extracted with DCM (2×10 mL). The combined organic layers were washed with brine (3×20 mL), dried over anhydrous Na₂SO₄, filtered and concentrated in vacuo to give the title compound (90.0 mg, 92% yield) as yellow oil. LC-MS (ESI⁺) m/z 447.0 (M+H)⁺.

### Step 4—N-[6-fluoro-2-(2-iodoethyl)indazol-5-yl]-6-(trifluoromethyl)pyridine-2-carboxamide

To a solution of 2-[6-fluoro-5-[[6-(trifluoromethyl)pyridine-2-carbonyl]amino]indazol-2-yl]ethyl methanesulfonate (90.0 mg, 201 umol) in THF (5.00 mL) was added NaI (181 mg, 1.21 mmol), and the mixture was stirred at 50° C. for 16 hrs. On completion, the reaction mixture was quenched by the addition of water (10 mL), and extracted with EA (2×10 mL). The mixture was then washed with brine (2×20 mL), dried over anhydrous Na₂SO₄, filtered and concentrated in vacuo to give a residue. The crude product was triturated with PE/EA(20/1) (10 mL) for 30 min to give the title compound (75.0 mg, 77% yield) as a yellow solid. ¹H NMR (400 MHz, DMSO-d₆) δ 10.25 (s, 1H), 8.85 (d, J=7.6 Hz, 1H), 8.52 (d, J=7.6 Hz, 1H), 8.16 (t, J=8.0 Hz, 1H), 8.00 (s, 1H), 7.90 (d, J=7.2 Hz, 1H), 7.43 (d, J=11.6 Hz, 1H), 4.74 (t, J=6.8 Hz, 2H), 3.69 (t, J=6.8 Hz, 2H). LC-MS (ESI⁺) m/z 478.9 (M+H)⁺.

### N-[2-(2-iodoethyl)-6-methoxy-indazol-5-yl]-6-(trifluoromethyl)pyridine-2-carboxamide (Intermediate BPK)

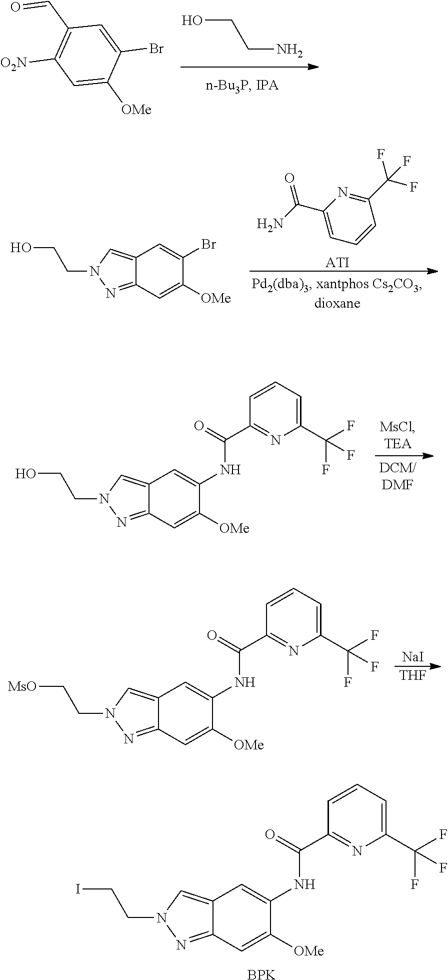

### Step 1—2-(5-Bromo-6-methoxy-indazol-2-yl)ethanol

After a solution of 5-bromo-4-methoxy-2-nitro-benzaldehyde (1.00 g, 3.85 mmol, synthesized via Steps 1-2 of Intermediate ATE) and 2-aminoethanol (281 mg, 4.61 mmol) in IPA (20 mL) was stirred at 80° C. for 2 hrs. Then the reaction mixture was cooled to 25° C., and tributylphosphane (2.33 g, 11.5 mmol) was added and the reaction mixture was stirred at 80° C. for 16 hrs. On completion, the reaction mixture was concentrated in vacuo. The residue was purified by silica gel chromatography (PE:EA=2:1) to give the title compound (650 mg, 62% yield) as white solid. ¹H NMR (400 MHz, CDCl₃) δ 7.89-7.83 (m, 2H), 7.01 (s, 1H), 4.50-4.45 (m, 2H), 4.14-4.06 (m, 2H), 3.95 (s, 3H), 3.36 (t, J=5.6 Hz, 1H).

### Step 2—N-[2-(2-hydroxyethyl)-6-methoxy-indazol-5-yl]-6-(trifluoromethyl)pyridine-2-carboxamide

A mixture of 2-(5-bromo-6-methoxy-indazol-2-yl)ethanol (300 mg, 1.11 mmol), 6-(trifluoromethyl) pyridine-2-carboxamide (231 mg, 1.22 mmol, Intermediate ATI), BrettPhos Pd G₃ (100 mg, 110 umol) and Cs₂CO₃ (721 mg, 2.21 mmol) in DMA (7 mL) was stirred at 80° C. for 16 hrs. On completion, the reaction mixture was filtered, the filter cake was washed with MeOH (20 mL), and the filtrate was concentrated in vacuo. The residue was purified by reverse phase (0.1% FA) to give the title compound (300 mg, 71% yield) as yellow solid. ¹H NMR (400 MHz, DMSO-d₆) δ 10.50 (s, 1H), 8.68 (s, 1H), 8.49-8.43 (m, 1H), 8.43-8.37 (m, 1H), 8.27 (s, 1H), 8.21 (dd, J=0.8, 7.6 Hz, 1H), 7.14 (s, 1H), 4.97 (t, J=5.2, 1H), 4.38 (t, J=5.6 Hz, 2H), 3.98 (s, 3H), 3.85 (d, J=5.6 Hz, 2H).

### Step 3—2-[6-Methoxy-5-[[6-(trifluoromethyl)pyridine-2-carbonyl]amino]indazol-2-yl]ethyl methane sulfonate

To a solution of N-[2-(2-hydroxyethyl)-6-methoxy-indazol-5-yl]-6-(trifluoromethyl)pyridine-2-carboxamide (200 mg, 525 umol) and TEA (106 mg, 1.05 mmol) in a mixed solvents of DMF (2 mL) and DCM (5 mL) was added MsCl (90.3 mg, 788 umol) at 0° C. The reaction mixture was stirred at 0-25° C. for 2 hrs. On completion, the reaction mixture was diluted with water (2 mL) and extracted with DCM (3×15 mL). The combined organic layers were washed with brine (2×10 mL), dried over Na₂SO₄, filtered and concentrated in vacuo to give the title compound (230 mg, 95% yield) as yellow solid. LC-MS (ESI⁺) m/z 459.2 (M+H)⁺.

### Step 4—N-[2-(2-iodoethyl)-6-methoxy-indazol-5-yl]-6-(trifluoromethyl)pyridine-2-carboxamide

A solution of 2-[6-methoxy-5-[[6-(trifluoromethyl)pyridine-2-carbonyl]amino]indazol-2-yl]ethyl methanesulfonate (230 mg, 501 umol) and NaI (376 mg, 2.51 mmol) in THF (6 mL) was stirred at 65° C. for 16 hrs. On completion, the reaction mixture was diluted with water (2 mL) and extracted with EA (3×15 mL). The combined organic layers were washed with brine (2×10 mL), dried over Na₂SO₄, filtered and concentrated in vacuo to give the title compound (230 mg, 95% yield) as yellow solid. LC-MS (ESI⁺) m/z 491.1 (M+H)⁺.

### Pyrazolo[1,5-a]pyrimidine-3-carboxamide (Intermediate AWV)

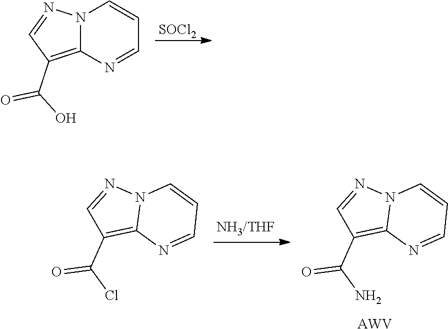

### Step 1—Pyrazolo[1,5-a]pyrimidine-3-carbonyl chloride

A mixture of pyrazolo[1,5-a]pyrimidine-3-carboxylic acid (1.00 g, 6.13 mmol, CAS #25940-35-6) in SOCl₂ (32.8 g, 276 mmol, 20 mL) was heated at 100° C. for 2 h. On completion, the reaction mixture was concentrated in vacuo to give the title compound (1.00 g, 90% yield) as a yellow solid. ¹H NMR (400 MHz, DMSO-d₆) δ 9.30 (dd, J=1.6, 6.8 Hz, 1H), 8.85 (dd, J=1.6, 4.0 Hz, 1H), 8.66 (s, 1H), 7.31 (dd, J=4.0, 7.2 Hz, 1H), 3.83 (s, 3H).

### Step 2—Pyrazolo[1,5-a]pyrimidine-3-carboxamide

A mixture of pyrazolo[1,5-a]pyrimidine-3-carbonyl chloride (500 mg, 2.75 mmol) in NH₃/THF (8.0 mL) was stirred at 25° C. for 5 hr. On completion, the reaction mixture was concentrated in vacuo to give the title compound (500 mg, 90% yield) as yellow solid. ¹H NMR (400 MHz, DMSO-d₆) δ 9.31 (dd, J=1.6, 7.2 Hz, 1H), 8.81 (dd, J=1.6, 4.0 Hz, 1H), 8.57 (s, 1H), 7.57-7.28 (m, 2H), 7.27 (dd, J=4.4, 7.2 Hz, 1H).

### N-[2-(2-iodoethyl)-6-methoxy-indazol-5-yl]pyrazolo[1,5-a]pyrimidine-3-carboxamide (Intermediate BPL)

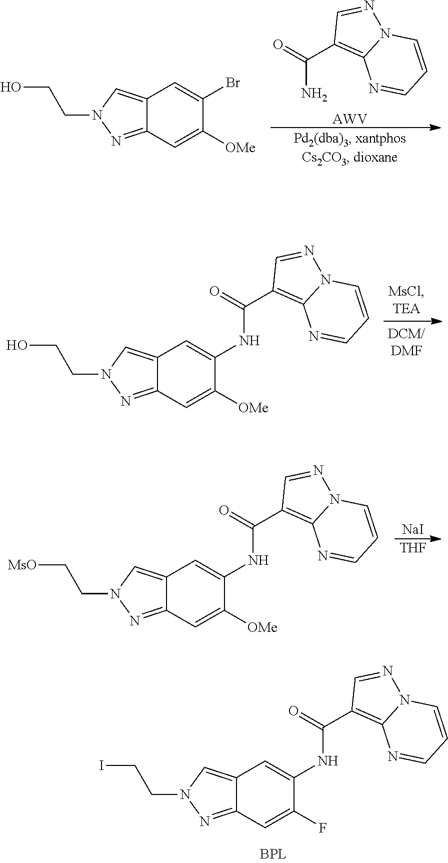

### Step 1—N-[2-(2-hydroxyethyl)-6-methoxy-indazol-5-yl]pyrazolo[1,5-a]pyrimidine-3-carboxamide

A mixture of 2-(5-bromo-6-methoxy-indazol-2-yl)ethanol (300 mg, 1.11 mmol, synthesized from Step 1 of Intermediate BPK), pyrazolo[1,5-a]pyrimidine-3-carboxamide (197 mg, 1.22 mmol, Intermediate AWV), BrettPhos Pd G₃ (100 mg, 110 umol) and Cs₂CO₃ (721 mg, 2.21 mmol) in DMA (7 mL) was stirred at 80° C. for 16 hrs. On completion, the reaction mixture was filtered, the filter cake was washed with MeOH (20 mL) and filtrate was concentrated in vacuo. The residue was purified by reverse phase (0.1% FA) to give the title compound (180 mg, 45% yield) as brown solid. ¹H NMR (400 MHz, DMSO-d₆) δ 10.53 (s, 1H), 9.36 (dd, J=1.6, 6.8 Hz, 1H), 8.95 (dd, J=1.6, 4.4 Hz, 1H), 8.69 (d, J=9.6 Hz, 2H), 8.22 (s, 1H), 7.34 (dd, J=4.4, 7.2 Hz, 1H), 7.09 (s, 1H), 5.07-4.83 (m, 1H), 4.36 (t, J=5.6 Hz, 2H), 4.02 (s, 3H), 3.87-3.82 (m, 2H).

### Step 2—2-[6-Methoxy-5-(pyrazolo[1,5-a]pyrimidine-3-carbonylamino)indazol-2-yl]ethyl methanesulfonate

To a solution of N-[2-(2-hydroxyethyl)-6-methoxy-indazol-5-yl]pyrazolo[1,5-a]pyrimidine-3-carboxamide (140 mg, 397 umol) and TEA (100 mg, 993 umol) in a mixed solvents of DMF (2 mL) and DCM (6 mL) was added MsCl (91.0 mg, 794 umol) at 0° C. The reaction mixture was stirred at 0° C. for 1 hr. On completion, the reaction mixture was quenched with water (2 mL) and extracted with DCM (3×20 mL). The combined organic layers were washed with brine (2×10 mL), dried over Na₂SO₄, filtered and concentrated in vacuo to give the title compound (160 mg, 93% yield) as yellow solid. LC-MS (ESI⁺) m/z 431.3 (M+H)⁺.

### Step 3—N-[2-(2-iodoethyl)-6-methoxy-indazol-5-yl]pyrazolo[1,5-a]pyrimidine-3-carboxamide

To a solution of 2-[6-methoxy-5-(pyrazolo[1,5-a]pyrimidine-3-carbonylamino)indazol-2-yl]ethyl methanesulfonate (160 mg, 371 umol) in THF (6 mL) was added NaI (278 mg, 1.86 mmol). The reaction mixture was stirred at 65° C. for 16 hrs. On completion, the reaction mixture was diluted with water (10 mL) and extracted with EA (3×30 mL). The combined organic layers were washed with brine (2×15 mL), dried over Na₂SO₄, filtered and concentrated in vacuo to give the title compound (160 mg, 93% yield) as yellow solid. LC-MS (ESI⁺) m/z 463.2 (M+H)⁺.

### Tert-butyl N-[6-fluoro-2-(2-iodoethyl)indazol-5-yl]carbamate (Intermediate BPM)

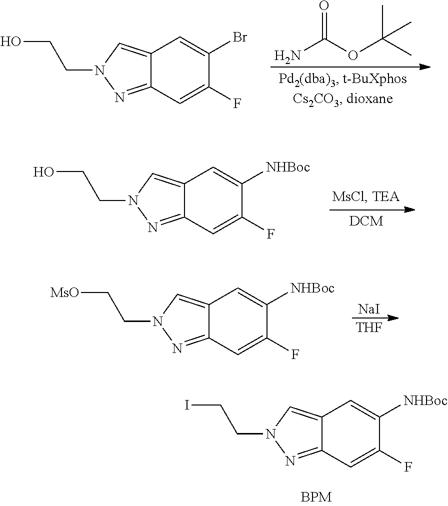

### Step 1—Tert-butyl N-[6-fluoro-2-(2-hydroxyethyl)indazol-5-yl]carbamate

A mixture of 2-(5-bromo-6-fluoro-indazol-2-yl)ethanol (300 mg, 1.16 mmol, synthesized via Step 1 of Intermediate BPJ), tert-butyl carbamate (271 mg, 2.32 mmol, 73.8 uL), t-Bu Xphos (98.3 mg, 231 umol), Pd₂(dba)₃ (106 mg, 115 umol) and Cs₂CO₃ (755 mg, 2.32 mmol) in dioxane (6.00 mL) was degassed and purged with N₂ three times. Then the mixture was stirred at 80° C. for 16 hrs under N₂ atmosphere. On completion, the reaction mixture was filtered and the filtrate was concentrated in vacuo to give a residue. The residue was purified by reversed phase (0.1% FA condition) to give the title compound (260 mg, 76% yield) as a white solid. ¹H NMR (400 MHz, DMSO-d₆) δ 8.74 (s, 1H), 8.30 (s, 1H), 7.79 (d, J=7.2 Hz, 1H), 7.35 (d, J=11.6 Hz, 1H), 4.98-4.94 (m, 1H), 4.40 (t, J=5.6 Hz, 2H), 3.86-3.81 (m, 2H), 1.45 (s, 9H). LC-MS (ESI⁺) m/z 296.1 (M+H)⁺.

### Step 2—2-[5-(Tert-butoxycarbonylamino)-6-fluoro-indazol-2-yl]ethyl methanesulfonate

To a solution of tert-butyl N-[6-fluoro-2-(2-hydroxyethyl)indazol-5-yl]carbamate (180 mg, 609 umol) and TEA (185 mg, 1.83 mmol, 254 uL) in DCM (10.0 mL) was added MsCl (139 mg, 1.22 mmol, 94.4 uL) at 0° C., then the mixture was stirred at 25° C. for 1 hr. On completion, the reaction mixture was quenched by addition ice water (0.5 mL), and then washed with brine (3×10 mL), dried over anhydrous Na₂SO₄, filtered and concentrated in vacuo to give the title compound (200 mg, 87% yield) as a yellow oil. LC-MS (ESI⁺) m/z 374.1 (M+H)⁺.

### Step 3—Tert-butyl N-[6-fluoro-2-(2-iodoethyl)indazol-5-yl]carbamate

To a solution of 2-[5-(tert-butoxycarbonylamino)-6-fluoro-indazol-2-yl]ethyl methanesulfonate (200 mg, 535 umol) in THF (5.00 mL) was added NaI (481 mg, 3.21 mmol), and the reaction mixture was stirred at 50° C. for 16 hrs. On completion, the reaction mixture was quenched with water (10 mL), and then extracted with EA (2×10 mL). The combined organic layers were washed with brine (2×20 mL), dried over anhydrous Na₂SO₄, filtered and concentrated in vacuo to give a residue. The residue was purified by column chromatography (SiO₂, Petroleum ether/Ethyl acetate=8/1 to 6/1, PE:EA=5:1, Rf=4.0) to give the title compound (200 mg, 92% yield) as a white solid. ¹H NMR (400 MHz, CDCl₃-d) δ 8.30 (d, J=7.2 Hz, 1H), 7.88 (s, 1H), 7.33 (d, J=12.0 Hz, 1H), 6.78 (s, 1H), 4.70 (t, J=6.8 Hz, 2H), 3.66 (t, J=6.8 Hz, 2H), 1.55 (s, 9H). LC-MS (ESI⁺) m/z 406.3 (M+H)⁺.

### 3-[4-[4-[2-(5-Amino-6-fluoro-indazol-2-yl)ethyl-methyl-amino]-1-piperidyl]-3-methyl-2-oxo-benzimidazol-1-yl]piperidine-2,6-dione (Intermediate BPN)

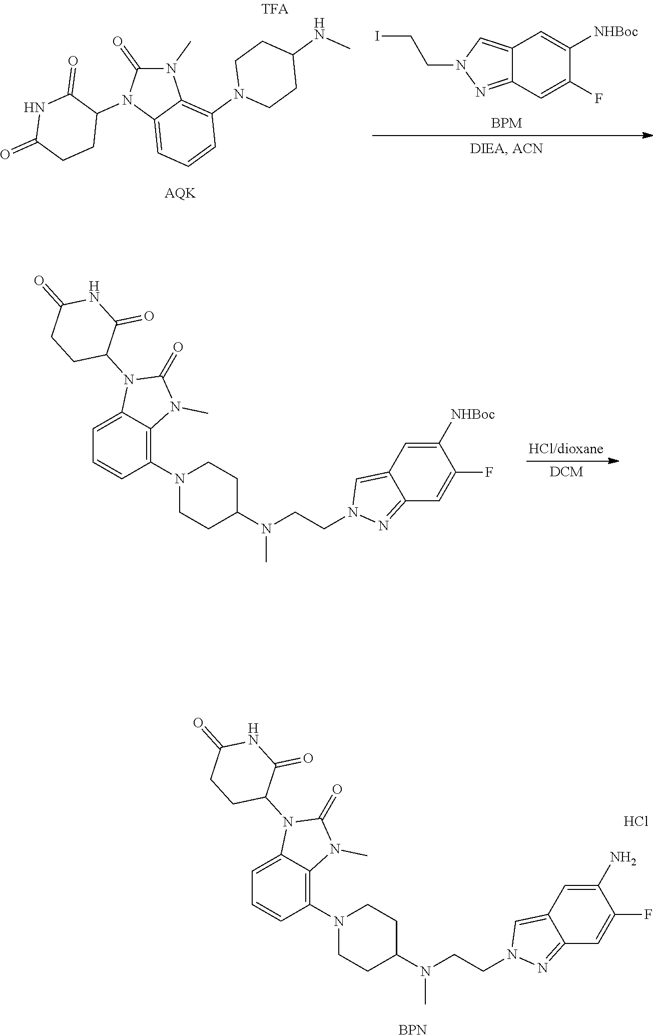

### Step 1—Tert-butyl N-[2-[2-[[1-[1-(2,6-dioxo-3-piperidyl)-3-methyl-2-oxo-benzimidazol-4-yl]-4-piperidyl]-methylamino]ethyl]-6-fluoro-indazol-5-yl]carbamate

To a solution of 3-[3-methyl-4-[4-(methylamino)-1-piperidyl]-2-oxo-benzimidazol-1-yl]piperidine-2,6-dione (227 mg, 468 umol, TFA, Intermediate AQK) and tert-butyl N-[6-fluoro-2-(2-iodoethyl)indazol-5-yl]carbamate (190 mg, 468 umol, Intermediate BPM) in ACN (6.00 mL) was added DIEA (181 mg, 1.41 mmol, 245 uL) at 25° C., and then the mixture was stirred at 80° C. for 16 hrs. On completion, the reaction mixture was quenched with ice water (0.5 mL), and then washed with brine (3×5 mL), dried over Na₂SO₄, filtered and concentrated in vacuo to give a residue. The residue was purified by reversed-phase (0.1% FA condition) to give the title compound (30.0 mg, 9.8% yield) as a white solid. ¹H NMR (400 MHz, DMSO-d₆) δ 11.17-11.02 (m, 1H), 8.73 (s, 1H), 8.35 (s, 1H), 7.79 (d, J=7.2 Hz, 1H), 7.37 (d, J=11.2 Hz, 1H), 6.98-6.92 (m, 1H), 6.85 (d, J=7.2 Hz, 2H), 5.41-5.24 (m, 1H), 4.45 (t, J=6.4 Hz, 2H), 3.59 (s, 3H), 3.08 (d, J=9.2 Hz, 2H), 2.96 (t, J=6.4 Hz, 2H), 2.90-2.84 (m, 1H), 2.73-2.65 (m, 2H), 2.63-2.57 (m, 2H), 2.45-2.40 (m, 1H), 2.29 (s, 3H), 2.01-1.95 (m, 1H), 1.71-1.65 (m, 2H), 1.60-1.50 (m, 2H), 1.45 (s, 9H). LC-MS (ESI⁺) m/z 649.3 (M+H)⁺.

### Step 2—3-[4-[4-[2-(5-Amino-6-fluoro-indazol-2-yl)ethyl-methyl-amino]-1-piperidyl]-3-methyl-2-oxo-benzimidazol-1-yl]piperidine-2,6-dione

To a solution of tert-butyl N-[2-[2-[[1-[1-(2,6-dioxo-3-piperidyl)-3-methyl-2-oxo-benzimidazol-4-yl]-4-piperidyl]-methylamino]ethyl]-6-fluoro-indazol-5-yl]carbamate (25.0 mg, 38.5 umol) in DCM (1.00 mL) was added HCl/dioxane (4 M, 0.50 mL). The mixture was stirred at 25° C. for 1 hr. On completion, the reaction mixture was concentrated in vacuo to give a residue. The residue was purified by reverse phase (0.1% HCl condition) to give the title compound (10.0 mg, 44% yield, HCl) as a yellow solid. LC-MS (ESI⁺) m/z 549.2 (M+H)⁺.

### 3-(4-Bromo-3,6-dimethyl-2-oxo-benzimidazol-1-yl)piperidine-2,6-dione (Intermediate

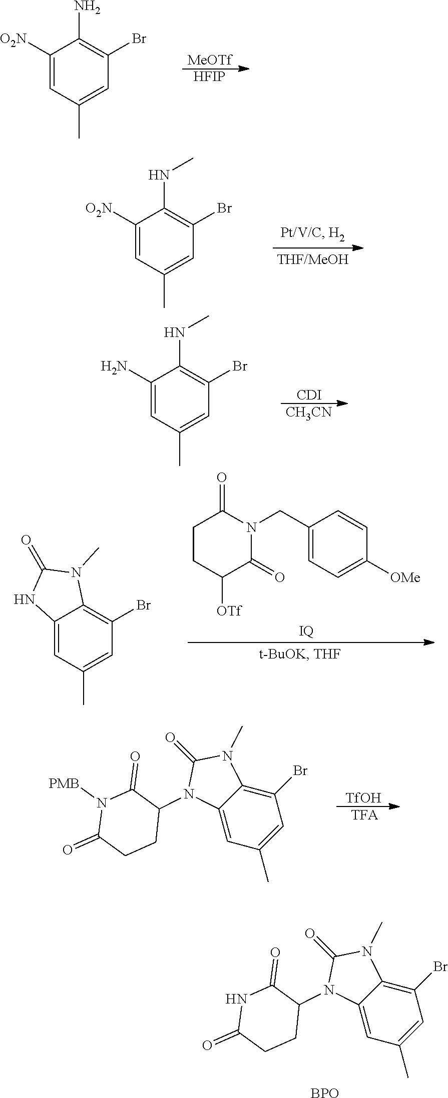

### Step 1—2-Bromo-N,4-dimethyl-6-nitro-aniline

To a mixture of 2-bromo-4-methyl-6-nitro-aniline (1.00 g, 4.33 mmol, CAS #827-24-7) in HFIP (10 mL) was added methyl trifluoromethanesulfonate (781 mg, 4.76 mmol, 520 uL) at 0° C. dropwise. The reaction mixture was stirred at 25° C. for 12 h. On completion, the reaction mixture was concentrated in vacuo. The residue was diluted with water (30 mL) and extracted with EA (2×30 mL). The combined organic layer was dried over Na₂SO₄, filtered and concentrated in vacuo to give a residue. The residue was purified by column chromatography to give the title compound (0.99 g, 93% yield) as yellow oil. ¹H NMR (400 MHz, CDCl₃) δ 7.70 (d, J=0.6 Hz, 1H), 7.54 (d, J=1.6 Hz, 1H), 2.99 (s, 3H), 2.28 (s, 3H).

### Step 2—2,6-Dibromo-4-methoxy-aniline

To a mixture of 1,3-dibromo-5-methoxy-2-nitro-benzene (9.30 g, 29.9 mmol) in THF (40 mL) and MeOH (40 mL) was added Pt/V/C (5.00 g, 19.1 mmol). The reaction mixture was stirred at 25° C. for 12 hours under H₂ (15 PSI) atmosphere. On completion, the reaction mixture was filtered and concentrated in vacuo. The residue was purified by column chromatography to give the title compound (2.50 g, 29% yield) as white solid. ¹H NMR (400 MHz, DMSO-d₆) δ 7.11 (s, 2H), 4.83 (s, 2H), 3.67 (s, 3H).

### Step 3—4-Bromo-3,6-dimethyl-1H-benzimidazol-2-one

To a mixture of 3-bromo-N2,5-dimethyl-benzene-1,2-diamine (640 mg, 2.98 mmol) in ACN (10 mL) was added CDI (723 mg, 4.46 mmol). The reaction mixture was stirred at 90° C. for 12 h. On completion, the reaction mixture was concentrated in vacuo. Then residue was diluted with H₂O (50 mL) and filtered to give the title compound (660 mg, 92% yield) as white solid. ¹H NMR (400 MHz, DMSO-d₆) δ 11.07 (s, 1H), 6.98 (s, 1H), 6.78 (s, 1H), 3.51 (s, 3H), 2.27 (s, 3H); LC-MS (ESI⁺) m/z 241.1 (M+H)⁺.

### Step 4—3-(4-Bromo-3,6-dimethyl-2-oxo-benzimidazol-1-yl)-1-[(4-methoxyphenyl)methyl]piperidine-2,6-dione

To a mixture of 4-bromo-3,6-dimethyl-1H-benzimidazol-2-one (0.66 g, 2.74 mmol) and t-BuOK (460 mg, 4.11 mmol) in THF (10 mL) was added a solution of [1-[(4-methoxyphenyl)methyl]-2,6-dioxo-3-piperidyl]trifluoromethanesulfonate (1.57 g, 4.11 mmol, Intermediate IQ) in THF (10 mL) dropwise at 0° C. The reaction mixture was stirred at 0° C. for 2 h. On completion, the reaction mixture was acidified with FA until the pH=5-6, then the residue was extracted with EA (2×30 mL). The combined organic layer was dried over Na₂SO₄, filtered and concentrated in vacuo to give a residue. The residue was purified by column chromatography to give the title compound (1.10 g, 850% yield) as white solid. ¹H NMR (400 MHz, DMSO-d₆) δ 7.21 (d, J=8.4 Hz, 2H), 7.07 (s, 1H), 6.85 (d, J=8.4 Hz, 3H), 5.57-5.50 (m, 1H), 4.79 (d, J=3.2 Hz, 2H), 3.72 (s, 3H), 3.60 (s, 3H), 3.11-2.99 (m, 1H), 2.86-2.68 (m, 2H), 2.23 (s, 3H), 2.08-2.00 (m, 1H).

### Step 5—3-(4-Bromo-3,6-dimethyl-2-oxo-benzimidazol-1-yl)piperidine-2,6-dione

A mixture of 3-(4-bromo-3,6-dimethyl-2-oxo-benzimidazol-1-yl)-1-[(4-methoxyphenyl)methyl]piperidine-2,6-dione (0.20 g, 423 umol) in TfOH (2 mL) and TFA (0.4 mL) was stirred at 70° C. for 12 hour. On completion, the residue was basified with TEA until the pH=5-6 and then the mixture was concentrated in vacuo. The residue was purified by reverse phase (0.1% FA condition) to give the title compound (100 mg, 67% yield) as white solid. ¹H NMR (400 MHz, DMSO-_{d6}) δ 11.21-10.96 (m, 1H), 7.08 (s, 1H), 7.02 (s, 1H), 5.41-5.35 (m, 1H), 3.60 (s, 3H), 2.95-2.83 (m, 1H), 2.75-2.58 (m, 2H), 2.29 (s, 3H), 2.05-1.96 (m, 1H); LC-MS (ESI⁺) m/z 354.1 (M+H)⁺.

### 3-[3,6-Dimethyl-2-oxo-4-(4-piperidyl)benzimidazol-1-yl]piperidine-2,6-dione (Intermediate BPP)

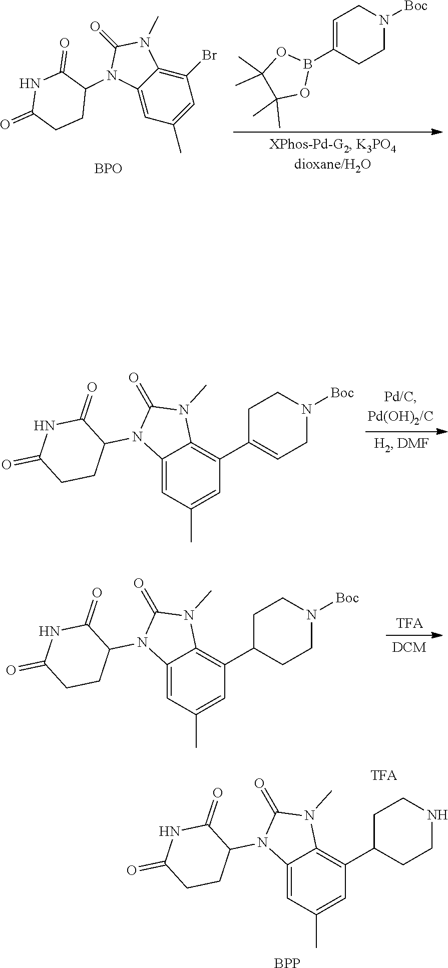

### Step 1—Tert-butyl 4-[1-(2,6-dioxo-3-piperidyl)-3,6-dimethyl-2-oxo-benzimidazol-4-yl]-3,6-dihydro-2H-pyridine-1-carboxylate

To a mixture of 3-(4-bromo-3,6-dimethyl-2-oxo-benzimidazol-1-yl)piperidine-2,6-dione (500 mg, 1.42 mmol, Intermediate BPO) tert-butyl 4-(4,4,5,5-tetramethyl-1,3,2-dioxaborolan-2-yl)-3,6-dihydro-2H-pyridine-1-carboxylate (877 mg, 2.84 mmol, CAS #286961-14-6) in dioxane (4 mL) and H₂O (0.2 mL) was added XPHOS-PD-G (111 mg, 141 umol) and K₃PO₄ (602 mg, 2.84 mmol). The reaction mixture was stirred at 80° C. for 3 h. On completion, the reaction mixture was filtered and concentrated in vacuo. The residue was purified by reverse phase (0.1% FA condition) to give the title compound (300 mg, 46% yield) as white solid. ¹H NMR (400 MHz, DMSO-d₆) δ 11.07 (s, 1H), 6.90 (s, 1H), 6.64 (s, 1H), 5.68 (s, 1H), 5.39-5.32 (m, 1H), 3.99 (s, 2H), 3.61-3.52 (m, 2H), 3.28 (s, 3H), 2.96-2.83 (m, 1H), 2.79-2.68 (m, 1H), 2.68-2.57 (m, 1H), 2.37 (s, 2H), 2.29 (s, 3H), 2.02-1.93 (m, 1H), 1.44 (s, 9H).

### Step 2—Tert-butyl 4-[1-(2,6-dioxo-3-piperidyl)-3,6-dimethyl-2-oxo-benzimidazol-4-yl]piperidine-1-carboxylate

To a mixture of tert-butyl 4-[1-(2,6-dioxo-3-piperidyl)-3,6-dimethyl-2-oxo-benzimidazol-4-yl]-3,6-dihydro-2H-pyridine-1-carboxylate (100 mg, 220 umol) in DMF (3 mL) was added Pd/C (50 mg, 10 wt %) and Pd(OH)₂/C (50 mg, 10 wt %). The reaction mixture was stirred at 40° C. for 48 hours under H₂ (50 PSI) atmosphere. On completion, the reaction mixture was filtered and the mixture was diluted with water (10 mL) and extracted with EA (3×20 mL). The combined organic layer was dried over Na₂SO₄, filtered and concentrated in vacuo to give the title compound (100 mg, 99% yield) as light yellow solid. ¹H NMR (400 MHz, DMSO-d₆) δ 11.07 (s, 1H), 6.82 (s, 1H), 6.80 (s, 1H), 5.38-5.30 (m, 1H), 4.15-4.02 (m, 2H), 3.56 (s, 3H), 3.42-3.35 (m, 1H), 2.95-2.89 (m, 1H), 2.85 (d, J=5.2 Hz, 1H), 2.72-2.67 (m, 1H), 2.65-2.56 (m, 1H), 2.52 (s, 1H), 2.28 (s, 3H), 2.01-1.92 (m, 1H), 1.79 (d, J=11.6 Hz, 2H), 1.63-1.51 (m, 2H), 1.42 (s, 9H).

### Step 3—3-[3,6-Dimethyl-2-oxo-4-(4-piperidyl)benzimidazol-1-yl]piperidine-2,6-dione

To a mixture of tert-butyl 4-[1-(2,6-dioxo-3-piperidyl)-3,6-dimethyl-2-oxo-benzimidazol-4-yl]piperidine-1-carboxylate (50.0 mg, 109 umol) in DCM (3 mL) was added TFA (1.54 g, 13.5 mmol) and the reaction mixture was stirred at 25° C. for 1 hour. On completion, the reaction mixture was concentrated in vacuo to give the title compound (50.0 mg, 97% yield, TFA) as red oil. LC-MS (ESI⁺) m/z 357.2 (M+H)⁺.

### N-(6-chloro-2-((1r,4r)-4-formylcyclohexyl)-2H-indazol-5-yl)-6-(trifluoromethyl)picolinamide (Intermediate BPQ)

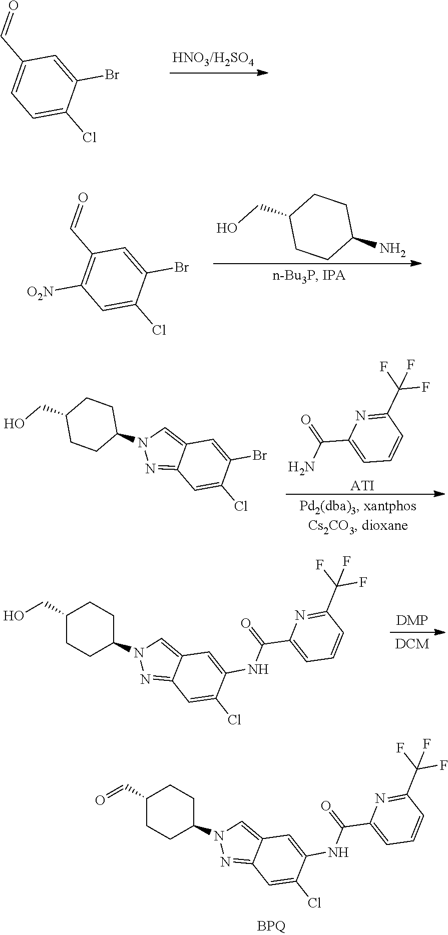

### Step 1—5-Bromo-4-chloro-2-nitrobenzaldehyde

To a solution of NaNO₃ (426 mg, 5.01 mmol) in H₂SO₄ (15 mL, 98% solution) was added 3-bromo-4-chloro-benzaldehyde (1.00 g, 4.56 mmol, CAS #86265-88-5) at 0° C. The mixture was stirred at 20° C. for 12 hours. On completion, the reaction mixture was poured into ice water (100 mL) at 0° C., and then diluted with H₂O (50 mL) and extracted with EA (3×50 mL). The combined organic layers were washed with brine (50 mL), dried over Na₂SO₄, filtered and concentrated in vacuo to give a residue. The residue was purified by column chromatography (SiO₂, Petroleum ether/Ethyl acetate=0/1 to 50/1) to give the title compound (650 mg, 50% yield) as a yellow solid. ¹H NMR (400 MHz, CDCl₃) δ 10.40 (s, 1H), 8.24 (s, 1H), 8.23 (s, 1H); LC-MS (ESI⁺) m/z 263.9 (M+H)⁺.

### Step 2—((1R,4r)-4-(5-bromo-6-chloro-2H-indazol-2-yl)cyclohexyl)methanol

A solution of 5-bromo-4-chloro-2-nitro-benzaldehyde (650 mg, 2.46 mmol) and (4-aminocyclohexyl) methanol (317 mg, 2.46 mmol, CAS #1467-84-1) in IPA (7 mL) was stirred at 80° C. for 3 hours. Then tributylphosphane (1.49 g, 7.37 mmol) was added and the mixture was stirred at 80° C. for 9 hours. On completion, the reaction mixture was concentrated under reduced pressure to give a residue. The residue was purified by column chromatography (SiO₂, Petroleum ether/Ethyl acetate=10/1 to 1/1) to give the title compound (600 mg, 70% yield) as a white solid. ¹H NMR (400 MHz, CDCl₃) δ 7.98 (s, 1H), 7.91 (s, 1H), 7.86 (s, 1H), 4.38 (m, 1H), 3.57 (m, 2H), 2.42-2.28 (m, 2H), 2.10-2.03 (m, 2H), 2.02-1.90 (m, 2H), 1.68 (m, 1H), 1.26 (m, 2H); LC-MS (ESI⁺) m/z 343.9 (M+H)⁺.

### Step 3—N-(6-chloro-2-((1r,4r)-4-(hydroxymethyl)cyclohexyl)-2H-indazol-5-yl)-6-(trifluoromethyl) picolinamide

To a solution of [4-(5-bromo-6-chloro-indazol-2-yl)cyclohexyl]methanol (200 mg, 581 umol) and 6-(trifluoromethyl)pyridine-2-carboxamide (110 mg, 581 umol, Intermediate ATI) in dioxane (4 mL) was added Pd₂(dba)₃ (53.2 mg, 58.20 umol), Cs₂CO₃ (379 mg, 1.16 mmol) and Xantphos (67.3 mg, 116 umol). The mixture was stirred at 80° C. for 12 hours. On completion, the reaction mixture was filtered and concentrated under reduced pressure to give a residue. The residue was purified by column chromatography (SiO₂, Petroleum ether/Ethyl acetate=5/1 to 1/1) to give the title compound (240 mg, 91% yield) as an off-white solid. ¹H NMR (400 MHz, CDCl₃) δ 10.71 (s, 1H), 8.91 (s, 1H), 8.51 (d, J=7.6 Hz, 1H), 8.15 (t, J=7.6 Hz, 1H), 7.98 (s, 1H), 7.90 (d, J=7.6 Hz, 1H), 7.85 (s, 1H), 4.40 (tt, J=4.4, 11.6 Hz, 1H), 3.57 (d, J=6.0 Hz, 2H), 2.41-2.31 (m, 2H), 2.12-1.93 (m, 4H), 1.78-1.65 (m, 1H), 1.33-1.21 (m, 2H); LC-MS (ESI⁺) m/z 453.3 (M+H)⁺.

### Step 4—N-(6-chloro-2-((1r,4r)-4-formylcyclohexyl)-2H-indazol-5-yl)-6-(trifluoromethyl)picolin amide

To a solution of N-[6-chloro-2-[4-(hydroxymethyl)cyclohexyl]indazol-5-yl]-6-(trifluoromethyl) pyridine-2-carboxamide (220 mg, 485 umol) in DCM (3 mL) was added DMP (309 mg, 728 umol). The mixture was stirred at 20° C. for 0.5 hour. On completion, the reaction mixture was quenched with sat. NaHCO₃ (20 mL) and sat. Na₂SO₃ (20 mL) at 0° C., and then diluted with H₂O (10 mL) and extracted with EA (3×50 mL). The combined organic layers were washed with brine (20 mL), dried over Na₂SO₄, filtered and concentrated under reduced pressure to give the title compound (200 mg, 89% yield) as a yellow solid. ¹H NMR (400 MHz, CDCl₃) δ 10.72 (s, 1H), 9.74 (s, 1H), 8.92 (s, 1H), 8.51 (d, J=7.6 Hz, 1H), 8.15 (t, J=7.6 Hz, 1H), 7.97 (s, 1H), 7.90 (d, J=7.68 Hz, 1H), 7.85 (s, 1H), 4.40 (tt, J=4.0, 11.2 Hz, 1H), 2.48-2.37 (m, 3H), 2.29 (d, J=12.0 Hz, 2H), 2.14-1.99 (m, 2H), 1.56-1.48 (m, 2H).

### 3-(4-Bromo-7-fluoro-3-methyl-2-oxo-benzimidazol-1-yl)piperidine-2,6-dione (Intermediate BPR)

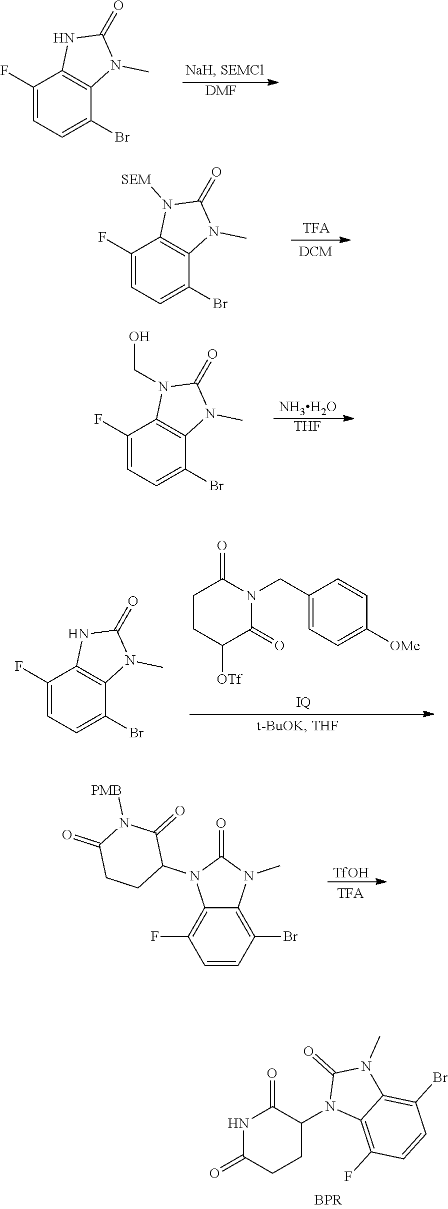

### Step 1—7-bromo-4-fluoro-1-methyl-3-(2-trimethylsilylethoxymethyl)benzimidazol-2-one

To a solution of 4-bromo-7-fluoro-3-methyl-1H-benzimidazol-2-one (8.7 g, 35.5 mmol, synthesized via Steps 1-4 of Intermediate BPT) in THF (20 mL) was added SEM-Cl (8.88 g, 53.2 mmol), and the mixture was stirred at 0° C. for 0.5 hour. Then to the mixture was added NaH (2.13 g, 53.2 mmol, 60% dispersion in mineral oil) in parts, then the mixture was stirred at 0° C. for 1 hour. On completion, the mixture was quenched by aq. NH₄Cl (40 mL) slowly. Then the mixture was extracted by EA (2×150 mL) and the combined organic layer was dried over by Na₂SO₄, and concentrated in vacuo to give the crude product. The crude product was purified by column chromatography on silica gel (PE:EA=50:1-8:1) to give as white solid (7.6 g, 57% yield). ¹H NMR (400 MHz, DMSO-d₆) δ 7.24 (dd, J=4.4, 9.0 Hz, 1H), 6.95 (dd, J=9.2, 10.6 Hz, 1H), 5.27 (s, 2H), 3.62 (s, 3H), 3.55 (t, J=7.8 Hz, 2H), 0.83 (t, J=7.8 Hz, 2H), −0.01 (s, 1H), −0.08 (s, 8H); LC-MS (ESI⁺) m/z 347.1 (M+H)⁺.

### Step 2—4-Bromo-7-fluoro-1-(hydroxymethyl)-3-methyl-benzimidazol-2-one

To a solution of 7-bromo-4-fluoro-1-methyl-3-(2-trimethylsilylethoxymethyl)benzimidazol-2-one (2.00 g, 5.33 mmol) in DCM (20 mL) was added TFA (28.0 g, 245 mmol, 18 mL) and the reaction mixture was stirred at 25° C. for 1 hrs. On completion, the reaction mixture was concentrated in vacuo to give the title compound (1.47 g, 100% yield) as a white solid. LC-MS (ESI⁺) m/z 257.2 (M−17)⁺.

### Step 3—4-Bromo-7-fluoro-3-methyl-1H-benzimidazol-2-one

To a solution of 4-bromo-7-fluoro-1-(hydroxymethyl)-3-methyl-benzimidazol-2-one (1.47 g, 5.34 mmol) in THF (10 mL) was added NH₃·H₂O (64.9 mmol, 10 mL, 25% solution) and the reaction mixture was stirred at 25° C. for 1 hrs. On completion, the reaction mixture was triturated with H₂O at rt for 30 min, then filtered and dried under vacuum to give the title compound (1.25 g, 76% yield, 80% purity) as a white solid. LC-MS (ESI⁺) m/z 247.2 (M+3)⁺.

### Step 4—3-(4-Bromo-7-fluoro-3-methyl-2-oxo-benzimidazol-1-yl)-1-[(4-methoxyphenyl)methyl]piperidine-2,6-dione

To a solution of 4-bromo-7-fluoro-3-methyl-1H-benzimidazol-2-one (1.25 g, 4.08 mmol) in THF (15 mL) was added t-BuOK (915 mg, 8.16 mmol) at 0° C. The mixture was then stirred at 0-25° C. for 0.5 hr. Then [1-[(4-methoxyphenyl)methyl]-2,6-dioxo-3-piperidyl]trifluoromethanesulfonate (2.33 g, 6.12 mmol, Intermediate IQ) was added to above mixture at 0° C. Next, the reaction mixture was stirred at 0-25° C. for 2.5 hrs. On completion, the reaction mixture was concentrated in vacuo to give a residue. Then the mixture was diluted with H₂O 60 mL and extracted with EA 15 mL (3×20 mL). The combined organic layers were washed with brine 20 mL (2×10 mL), dried over by Na₂SO₄, filtered and the filtrate was concentrated in vacuo. The residue was purified by reverse phase (0.1% FA condition) to give the title compound (1.25 g, 64% yield) as a blue solid. ¹H NMR (400 MHz, DMSO-d₆) δ 7.27 (dd, J=4.4, 8.8 Hz, 1H), 7.19 (d, J=8.4 Hz, 3H), 6.84 (d, J=8.8 Hz, 2H), 5.74 (dd, J=5.2, 12.8 Hz, 1H), 4.82 (s, 1H), 3.72 (s, 3H), 3.69 (s, 3H), 3.21-3.11 (m, 1H), 2.88-2.70 (m, 2H), 2.28-2.12 (m, 2H); LC-MS (ESI⁺) m/z 498.4 (M+23)⁺.

### Step 5—3-(4-Bromo-7-fluoro-3-methyl-2-oxo-benzimidazol-1-yl)piperidine-2,6-dione

To a solution of 3-(4-bromo-7-fluoro-3-methyl-2-oxo-benzimidazol-1-yl)-1-[(4-methoxyphenyl) methyl]piperidine-2,6-dione (550 mg, 1.15 mmol) in TFA (5 mL) was added TfOH (11.3 mmol, 1 mL) at 25° C. Then the mixture was stirred at 65° C. for 2 hrs. On completion, the reaction mixture was concentrated in vacuo. The residue was purified by reverse phase (0.10% FA condition) to give the title compound (300 mg, 72% yield) as a green solid. LC-MS (ESI⁺) m/z 355.9 (M+H)⁺.

### 3-[7-Fluoro-3-methyl-2-oxo-4-(4-piperidyl)benzimidazol-1-yl]piperidine-2,6-dione (Intermediate BPS)

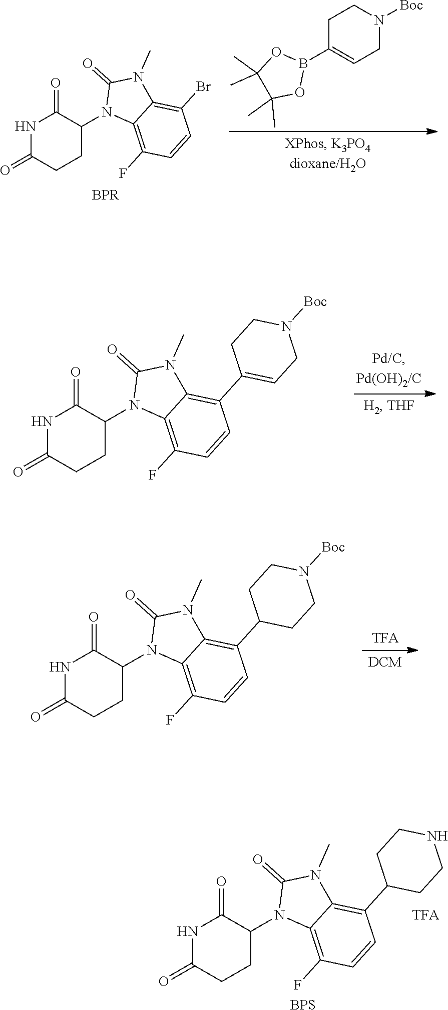

### Step 1—Tert-butyl 4-[1-(2,6-dioxo-3-piperidyl)-7-fluoro-3-methyl-2-oxo-benzimidazol-4-yl]-3,6-dihydro-2H-pyridine-1-carboxylate

To a solution of 3-(4-bromo-7-fluoro-3-methyl-2-oxo-benzimidazol-1-yl)piperidine-2,6-dione (270 mg, 758 umol, Intermediate BPR) and tert-butyl 4-(4,4,5,5-tetramethyl-1,3,2-dioxaborolan-2-yl)-3,6-dihydro-2H-pyridine-1-carboxylate (351 mg, 1.14 mmol, CAS #286961-14-6) in dioxane (5 mL) and H₂O (0.5 mL) was added XPHOS-PD-G2 (89.4 mg, 113 umol), K₃PO₄ (321 mg, 1.52 mmol) at 25° C. The reaction mixture was stirred at 80° C. for 6 hrs. On completion, the reaction mixture was filtered and the filtrate was concentrated in vacuo. The residue was purified by reverse phase (0.1% FA condition) to give the title compound (200 mg, 57% yield) as a white solid. ¹H NMR (400 MHz, DMSO-d₆) δ 11.18-11.01 (m, 1H), 6.97-6.87 (m, 1H), 6.86-6.81 (m, 1H), 5.76-5.67 (m, 1H), 5.58-5.48 (m, 1H), 3.99 (s, 2H), 3.57 (s, 2H), 3.35 (s, 3H), 3.08-2.92 (m, 1H), 2.65-2.58 (m, 1H), 2.37 (s, 2H), 2.30-2.16 (m, 1H), 2.14-2.03 (m, 1H), 1.43 (s, 9H); LC-MS (ESI⁺) m/z 459.4 (M+H)⁺.

### Step 2—Tert-butyl 4-[1-(2,6-dioxo-3-piperidyl)-7-fluoro-3-methyl-2-oxo-benzimidazol-4-yl]piperidine-1-carboxylate

To a solution of tert-butyl 4-[1-(2,6-dioxo-3-piperidyl)-7-fluoro-3-methyl-2-oxo-benzimidazol-4-yl]-3,6-dihydro-2H-pyridine-1-carboxylate (100 mg, 218 umol) in THF (2 mL) was added Pd/C (100 mg, 218 umol, 10 wt %) and Pd(OH)₂ (100 mg, 71.2 umol, 10 wt %) at 25° C. The reaction mixture was stirred at 25° C. for 3 hrs under H₂ (50 Psi). On completion, the reaction mixture was filtered with celite and the filtrate was concentrated in vacuo to give the title compound (70.0 mg, 69% yield) as a yellow solid. LC-MS (ESI⁺) m/z 405.1 (M−56)⁺.

### Step 3—3-[7-Fluoro-3-methyl-2-oxo-4-(4-piperidyl)benzimidazol-1-yl]piperidine-2,6-dione

To a solution of tert-butyl 4-[1-(2,6-dioxo-3-piperidyl)-7-fluoro-3-methyl-2-oxo-benzimidazol-4-yl]piperidine-1-carboxylate (70.0 mg, 152 umol) in DCM (1.5 mL) was added TFA (4.05 mmol, 0.3 mL) at 25° C. and the reaction mixture was stirred for 1 hr. On completion, the reaction mixture was concentrated in vacuo to give the title compound (70.0 mg, 97% yield) as yellow oil. LC-MS (ESI⁺) m/z 361.1 (M+H)⁺.

### 3-(4-bromo-7-fluoro-3-methyl-2-oxo-benzimidazol-1-yl)piperidine-2,6-dione (Intermediate BPT)

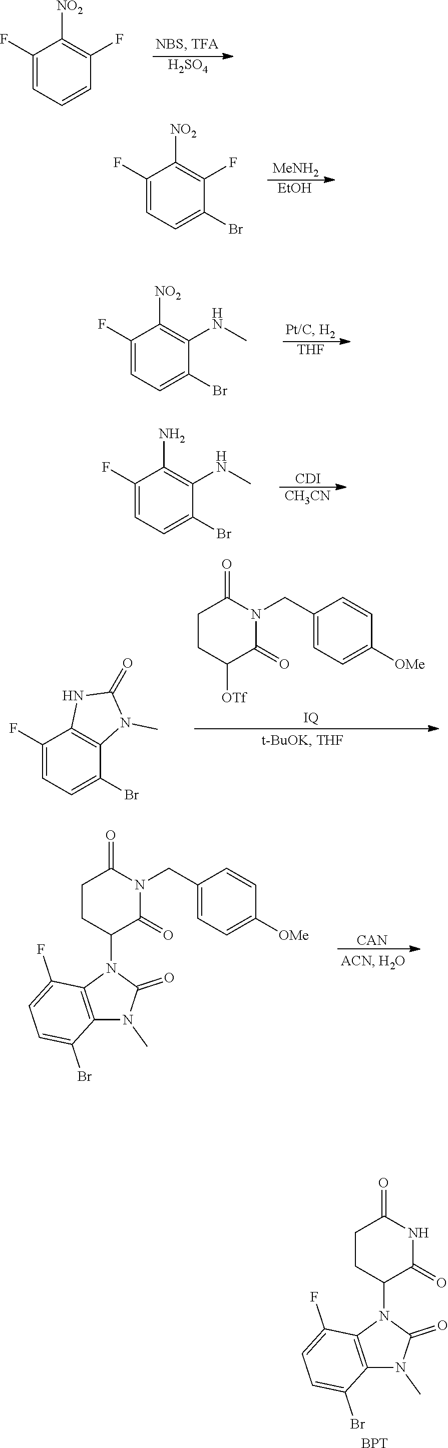

### Step 1—1-bromo-2,4-difluoro-3-nitro-benzene

To a solution of H₂SO₄ (10 mL) was added TFA (50 mL) at 0° C. slowly. Then to the mixture was added 1,3-difluoro-2-nitro-benzene (5 g, 31.4 mmol), then added N-bromosuccinimide (5.59 g, 31.4 mmol) slowly in portions at 0° C., and the reaction was stirred at 70° C. for 18 hours. Next, after cooling the mixture to 25° C., more N-bromosuccinimide (1.68 g, 9.43 mmol) was added and the reaction was stirred at 70° C. for the other 1 hour. On completion, after the mixture was cooled to 25° C., the mixture was diluted with ice water (80 mL), acidified with sat. aq. NaHCO₃ to pH=8, then extracted with EA (3×100 mL). The combined organic layer was washed with brine (30 mL), dried over Na₂SO₄, filtered and concentrated in vacuo to give the title compound (7.1 g, 94% yield) as brown oil. ¹H NMR (400 MHz, DMSO-d₆) δ 8.16 (m, 9.2 Hz, 1H), 7.54 (J=1.6, 9.6 Hz, 1H).

### Step 2—6-bromo-3-fluoro-N-methyl-2-nitro-aniline

To a solution of 1-bromo-2,4-difluoro-3-nitro-benzene (2 g, 8.40 mmol) in EtOH (6 mL) was added MeNH₂ (1.19 g, 12.6 mmol, 2 mL, 33% solution) at 25° C. and the mixture was stirred for 2 hours. On completion, the mixture was concentrated in vacuo, diluted with water (30 mL), then extracted with EA (3×90 mL). The combined organic layer was washed with brine (20 mL), dried over Na₂SO₄, filtered and concentrated in vacuo to give the crude product. The crude product was purified by column chromatography on silica gel (PE:EA=1:0-10:1) to give the title compound (1.8 g, 86% yield) as brown solid. ¹H NMR (400 MHz, DMSO-d₆) δ 7.71 (dd, J=6.0, 8.8 Hz, 1H), 6.67 (t, J=9.6 Hz, 1H), 6.27 (d, J=4.4 Hz, 1H), 2.70 (d, J=5.2 Hz, 3H).

### Step 3—3-bromo-6-fluoro-N2-methyl-benzene-1,2-diamine

To a solution of 6-bromo-3-fluoro-N-methyl-2-nitro-aniline (500 mg, 2.01 mmol) in THF (4 mL) was added platinum (68 mg, 34.8 umol, 10 wt %, CAS #7440-06-4) at 25° C. under H₂ (15 psi). The mixture was stirred at 25° C. for 16 hours. The mixture was filtered and the filter cake was washed with EA (50 mL). The filtrate and washing were combined and concentrated in vacuo. The residue was diluted with water (15 mL), then extracted with EA (3×30 mL). The combined organic layer was washed with brine (10 mL), dried over Na₂SO₄, filtered and concentrated in vacuo to give the title compound (420 mg, 95% yield) as light brown solid. ¹H NMR (400 MHz, DMSO-d₆) δ 6.79-6.62 (m, 2H), 4.96 (s, 2H), 3.90 (s, 1H), 2.61 (s, 3H); LC-MS (ESI⁺) m/z 219.0 (M+H)⁺.

### Step 4—4-bromo-7-fluoro-3-methyl-1H-benzimidazol-2-one

To a solution of 3-bromo-6-fluoro-N2-methyl-benzene-1,2-diamine (420 mg, 1.92 mmol) in ACN (5 mL) was added CDI (466 mg, 2.88 mmol) at 25° C. Then the mixture was stirred at 85° C. for 16 hours. On completion, after the reaction was cooled to rt, the mixture was concentrated in vacuo, and diluted with water (15 mL) and CH₃CN (3 mL). The mixture was filtered and the filter cake was washed with water (30 mL), then concentrated in vacuo to give the title compound (270 mg, 57% yield) as brown solid. ¹H NMR (400 MHz, DMSO-d₆) δ 11.75 (s, 1H), 7.15 (dd, J=4.8, 9.2 Hz, 1H), 6.88 (t, J=9.6 Hz, 1H), 3.56 (s, 3H); LC-MS (ESI⁺) m/z 247.0 (M+H)⁺.

### Step 5—3-(4-bromo-7-fluoro-3-methyl-2-oxo-benzimidazol-1-yl)-1-[(4-methoxyphenyl)methyl]piperidine-2,6-dione

To a solution of 4-bromo-7-fluoro-3-methyl-1H-benzimidazol-2-one (130 mg, 530 umol) in THF (3 mL) was added t-BuOK (65.4 mg, 583 umol) at 0° C. The mixture was stirred at 0° C. for 0.5 h. Next, to the mixture was added [1-[(4-methoxyphenyl) methyl]-2,6-dioxo-3-piperidyl]trifluoromethanesulfonate (263 mg, 689 umol, Intermediate IQ) in THF (5 mL) at 0° C. The mixture was stirred at 0° C. for other 1.5 hours. On completion, the mixture was concentrated in vacuo. The residue was purified by column chromatography on silica gel (PE:EA=50:1-10:1) to give the title compound (195 mg, 77% yield) as light brown solid. ¹H NMR (400 MHz, DMSO-d₆) δ 7.27 (dd, J=4.4, 9.2 Hz, 1H), 7.19 (d, J=8.4 Hz, 2H), 6.84 (d, J=8.8 Hz, 2H), 5.74 (dd, J=5.6, 12.8 Hz, 1H), 4.91-4.61 (m, 2H), 3.73-3.70 (m, 3H), 3.69 (s, 3H), 3.32 (s, 1H), 3.23-3.08 (m, 1H), 2.81 (d, J=18.0 Hz, 1H), 2.30-2.08 (m, 2H); LC-MS (ESI⁺) m/z 478.2 (M+H)⁺.

### Step 6—3-(4-bromo-7-fluoro-3-methyl-2-oxo-benzimidazol-1-yl)piperidine-2,6-dione

To a solution of 3-(4-bromo-7-fluoro-3-methyl-2-oxo-benzimidazol-1-yl)-1-[(4-methoxyphenyl) methyl]piperidine-2,6-dione (190 mg, 399 umol) in ACN (2 mL) was added CAN (1.09 g, 1.99 mmol,) in H₂O (1 mL) at 25° C. The mixture was stirred at 25° C. for 16 hours. On completion, the mixture was concentrated in vacuo to give the crude product. The crude product was purified by prep-HPLC (column: Phenomenex Synergi C18 150*25 mm*10 um; mobile phase:[water(0.225% FA)-ACN]; B %: 22%-52%, 10 min) to give the title compound (10.6 mg, 6.6% yield, FA salt) as white solid. ¹H NMR (400 MHz, DMSO-d₆) δ 11.14 (s, 1H), 7.27 (dd, J=4.8, 9.2 Hz, 1H), 7.06-6.88 (m, 1H), 5.56 (dd, J=5.2, 12.4 Hz, 1H), 3.68 (s, 3H), 3.06-2.93 (m, 1H), 2.62 (d, J=17.6 Hz, 1H), 2.28-2.05 (m, 2H).; LC-MS (ESI⁺) m/z 356.1 (M+H)⁺.

### 3-[6-Fluoro-3-methyl-2-oxo-4-(4-piperidyl)benzimidazol-1-yl]piperidine-2,6-dione (Intermediate BPU)

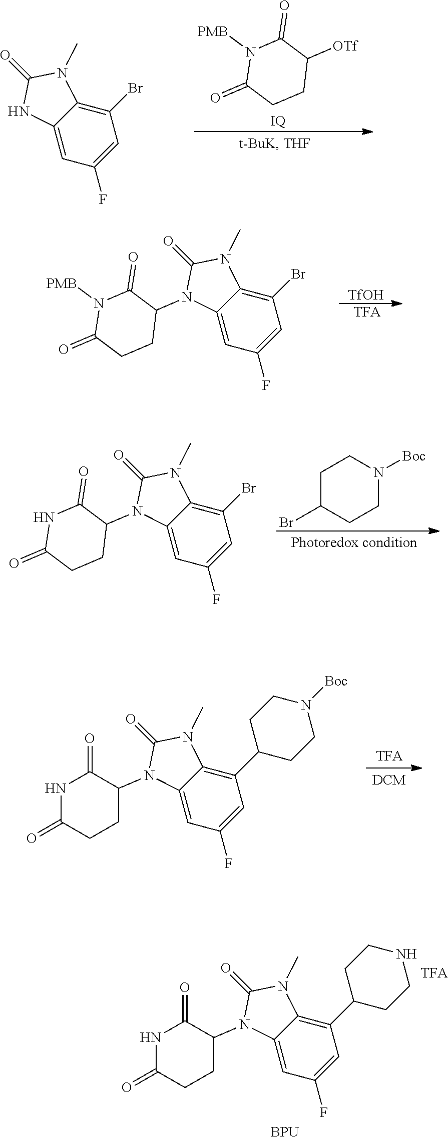

### Step 1—3-(4-Bromo-6-fluoro-3-methyl-2-oxo-benzimidazol-1-yl)-1-[(4-methoxyphenyl)methyl]piperidine-2,6-dione

To a solution of 4-bromo-6-fluoro-3-methyl-1H-benzimidazol-2-one (2 g, 8.16 mmol, synthesized via Steps 1-3 of Intermediate BQD) in THF (20 mL) was added t-BuOK (1.37 g, 12.2 mmol) at 0° C. The mixture was stirred at 0° C. for 0.5 hr, then a solution of [1-[(4-methoxyphenyl)methyl]-2,6-dioxo-3-piperidyl]trifluoromethanesulfonate (4.67 g, 12.2 mmol, Intermediate IQ) in THF (20 mL) was added dropwise to the mixture, and the reaction mixture was stirred at 0° C. for 3 hr. On completion, the mixture was acidified with FA to pH=3-5, diluted with water (100 mL), and extracted with EA (2×100 mL). The organic layer was washed with brine (100 mL), then concentrated in vacuo. The residue was purified by reverse phase (0.1% FA condition) to give the title compound (2.2 g, 56% yield) as a yellow solid. ¹H NMR (400 MHz, DMSO-d₆) δ 7.28-7.07 (m, 4H), 6.84 (d, J=8.8 Hz, 2H), 5.56 (dd, J=5.2, 12.8 Hz, 1H), 4.89-4.68 (m, 2H), 3.71 (s, 3H), 3.61 (s, 3H), 3.08-2.94 (m, 1H), 2.88-2.68 (m, 2H), 2.14-2.01 (m, 1H); LC-MS (ESI⁺) m/z 478.3 (M+3)⁺.

### Step 2—3-(4-Bromo-6-fluoro-3-methyl-2-oxo-benzimidazol-1-yl)piperidine-2,6-dione

To a solution of 3-(4-bromo-6-fluoro-3-methyl-2-oxo-benzimidazol-1-yl)-1-[(4-methoxy phenyl)methyl]piperidine-2,6-dione (1.60 g, 3.36 mmol) in TFA (10 mL) was added TfOH (4.25 g, 28.3 mmol), then the reaction mixture was stirred at 65° C. for 12 hrs. On completion, the reaction mixture was concentrated in vacuo to give a residue. The residue was purified by reverse phase (0.10% FA condition) to give the title compound (800 mg, 66% yield) as a yellow solid. 1H NMR (400 MHz, DMSO-d₆) δ 11.14 (s, 1H), 7.26 (dd, J=2.4, 8.8 Hz, 1H), 7.21 (dd, J=2.4, 9.6 Hz, 1H), 5.40 (dd, J=5.2, 12.4 Hz, 1H), 3.61 (s, 3H), 2.92-2.70 (m, 2H), 2.66-2.58 (m, 1H), 2.07-1.97 (m, 1H); LC-MS (ESI⁺) m/z 358.0 (M+3)⁺.

### Step 3—Tert-butyl 4-[1-(2,6-dioxo-3-piperidyl)-6-fluoro-3-methyl-2-oxo-benzimidazol-4-yl]piperidine-1-carboxyalate

To an 8 mL vial equipped with a stir bar was added 3-(4-bromo-6-fluoro-3-methyl-2-oxo-benzimidazol-1-yl)piperidine-2,6-dione (600 mg, 1.68 mmol), tert-butyl 4-bromopiperidine-1-carboxylate (578 mg, 2.19 mmol, CAS #180695-79-8), Ir[dF(CF₃)ppy]₂(dtbpy)(PF₆) (18.9 mg, 16.8 umol), NiCl₂.dtbbpy (3.35 mg, 8.42 umol), TTMSS (418 mg, 1.68 mmol, 519 uL), and 2,6-lutidine (361 mg, 3.37 mmol, 392 uL) in DME (10 mL). The vial was sealed and placed under nitrogen was added. The reaction was stirred and irradiated with a 34 W blue LED lamp (7 cm away), with cooling fan to keep the reaction temperature at 25° C. for 14 hr. On completion, the reaction mixture was filtered and the filtrate was concentrated in vacuo to give a residue. The residue was purified by reverse phase (0.10% FA condition) to give the title compound (90 mg, 11% yield) as yellow oil. LC-MS (ESI⁺) m/z 483.3 (M+Na)⁺.

### Step 4—3-[6-Fluoro-3-methyl-2-oxo-4-(4-piperidyl)benzimidazol-1-yl]piperidine-2,6-dione

To a solution of tert-butyl 4-[1-(2,6-dioxo-3-piperidyl)-6-fluoro-3-methyl-2-oxo-benzimidazol-4-yl]piperidine-1-carboxylate (40.0 mg, 86.8 umol) in DCM (1 mL) was added TFA (462 mg, 4.05 mmol, 0.3 mL), then the reaction mixture was stirred at 25° C. for 0.5 hr. On completion, the reaction mixture was concentrated in vacuo to give the title compound (40.0 mg, 97% yield, TFA) as a yellow solid. LC-MS (ESI⁺) m/z 361.3 (M+H)⁺.

### 3-(4-Bromo-6-fluoro-3-methyl-2-oxo-2,3-dihydro-1H-benzo[d]imidazol-1-yl)piperidine-2,6-dione (Intermediate BQD)

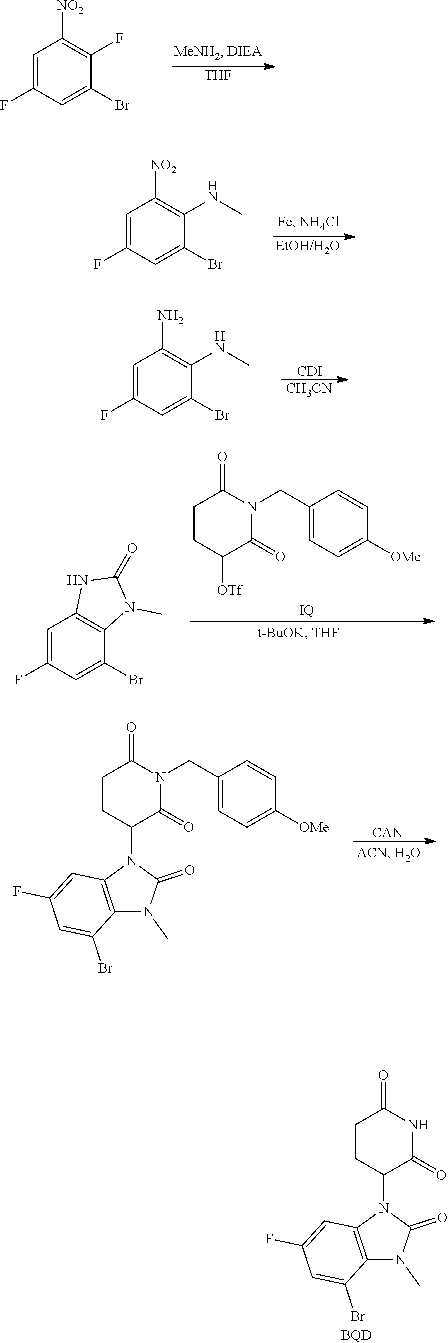

### Step 1—2-Bromo-4-fluoro-N-methyl-6-nitroaniline

To a solution of 1-bromo-2,5-difluoro-3-nitro-benzene (3.00 g, 12.6 mmol, CAS #741721-51-7) and DIEA (3.26 g, 25.2 mmol) in DMF (20 mL) was added a solution of MeNH₂ (2 M, 31.5 mL) in THF (30 mL). The mixture was stirred at 80° C. for 6 hrs. On completion, the reaction mixture was concentrated in vacuo. The residue was diluted with EA (100 mL) and washed with water (3×100 mL). The organic layer was dried over Na₂SO₄, filtered and concentrated in vacuo to give the title compound (3.10 g, 99% yield) as yellow oil. ¹H NMR (400 MHz, CDCl₃) δ 7.69 (dd, J=3.2, 8.2 Hz, 1H), 7.55 (dd, J=3.2, 7.1 Hz, 1H), 3.00 (s, 3H).

### Step 2—6-Bromo-4-fluoro-N1-methylbenzene-1,2-diamine

To a solution of 2-bromo-4-fluoro-N-methyl-6-nitro-aniline (3.10 g, 12.4 mmol) in the EtOH (50 mL) and H₂O (10 mL) was added Fe (3.48 g, 62.2 mmol) and NH₄Cl (6.66 g, 124 mmol) and the mixture was stirred at 80° C. for 3 hrs. On completion, the reaction mixture was filtered and concentrated in vacuo. Then the residue was diluted with EA (100 mL) and washed with water (3×100 mL). The organic layer was dried over Na₂SO₄, filtered and concentrated in vacuo to give the title compound (2.70 g, 98% yield) as yellow oil. ¹H NMR (400 MHz, CDCl₃) δ 6.77-6.58 (m, 1H), 6.46-6.32 (m, 1H), 4.17 (s, 2H), 2.68-2.00 (m, 3H).

### Step 3—7-Bromo-5-fluoro-1-methyl-1H-benzo[d]imidazol-2(3H)-one

To a solution 3-bromo-5-fluoro-N2-methyl-benzene-1,2-diamine (1.35 g, 6.16 mmol) in the CH₃CN (20 mL) was added CDI (2.00 g, 12.3 mmol) and the mixture was stirred at 90° C. for 12 hrs under N₂. On completion, the reaction mixture was concentrated in vacuo and purified by column chromatography (PE:EA=10:1 to 1:1) to give the title compound (0.85 g, 56% yield) as white solid. ¹H NMR (400 MHz, DMSO-d₆) δ 11.36 (s, 1H), 7.11 (dd, J=2.4, 9.8 Hz, 1H), 6.89 (dd, J=2.4, 8.4 Hz, 1H), 3.54 (s, 3H).

### Step 4—3-(4-Bromo-6-fluoro-3-methyl-2-oxo-2,3-dihydro-1H-benzo[d]imidazol-1-yl)-1-(4-methoxy benzyl)piperidine-2,6-dione

To a solution of 4-bromo-6-fluoro-3-methyl-1H-benzimidazol-2-one (800 mg, 3.26 mmol) in the THF (8 mL) was added tBuOK (549 mg, 4.90 mmol) at −5° C. and the mixture was stirred for 0.5 hr. Then a solution of [1-[(4-methoxyphenyl)methyl]-2,6-dioxo-3-piperidyl]trifluoromethanesulfonate (1.49 g, 3.92 mmol, Intermediate IQ) in THF (4 mL) was added dropwise and the mixture was stirred at −5° C. for 3 hrs. On completion, the reaction mixture was concentrated in vacuo. The residue was diluted with EA (100 mL) and washed with saturated NH₄Cl (50 mL). The organic layer was dried over Na₂SO₄, filtered and concentrated in vacuo. The residue was purified by reversed phase (TFA, 0.1%) to give the title compound (400 mg, 26% yield) as gray solid. ¹H NMR (400 MHz, CDCl₃) δ 7.36 (d, J=8.6 Hz, 2H), 7.00 (dd, J=2.3, 9.1 Hz, 1H), 6.84 (d, J=8.8 Hz, 2H), 6.20 (dd, J=2.3, 8.0 Hz, 1H), 5.21 (dd, J=5.5, 13.3 Hz, 1H), 4.97 (s, 2H), 3.80 (s, 3H), 3.77 (s, 3H), 3.06-2.99 (m, 1H), 2.89-2.84 (m, 1H), 2.56 (dq, J=4.6, 13.5 Hz, 1H), 2.22-2.12 (m, 1H).

### Step 5—3-(4-Bromo-6-fluoro-3-methyl-2-oxo-2,3-dihydro-1H-benzo[d]imidazol-1-yl)piperidine-2,6-dione

To a solution of 3-(4-bromo-6-fluoro-3-methyl-2-oxo-benzimidazol-1-yl)-1-[(4-methoxyphenyl) methyl]piperidine-2,6-dione (300 mg, 629 umol) in the MeCN (6 mL) was added a solution of CAN (1.73 g, 3.15 mmol) in the H₂O (1.5 mL) at 0° C. The mixture was stirred at 20° C. for 12 hrs. On completion, the reaction mixture was concentrated in vacuo and the purified by prep-HPLC (column: Phenomenex Synergi C18 150*25 mm*10 um; mobile phase: [water (0.225% FA)-ACN]; B %: 24%-57%, 11 min) to give the title compound (20.0 mg, 9% yield) as pink solid. ¹H NMR (400 MHz, DMSO-d₆) δ 11.14 (s, 1H), 7.26 (dd, J=2.2, 8.8 Hz, 1H), 7.22 (dd, J=2.2, 9.6 Hz, 1H), 5.40 (dd, J=5.4, 12.4 Hz, 1H), 3.62 (s, 3H), 2.90-2.77 (m, 1H), 2.76-2.70 (m, 1H), 2.68-2.62 (m, 1H), 2.07-1.98 (m, 1H); LC-MS (ESI⁺) m/z 355.9 (M+H)⁺.

### Methyl 5-chloro-2-methyl-4-nitrobenzoate (Intermediate BMM)

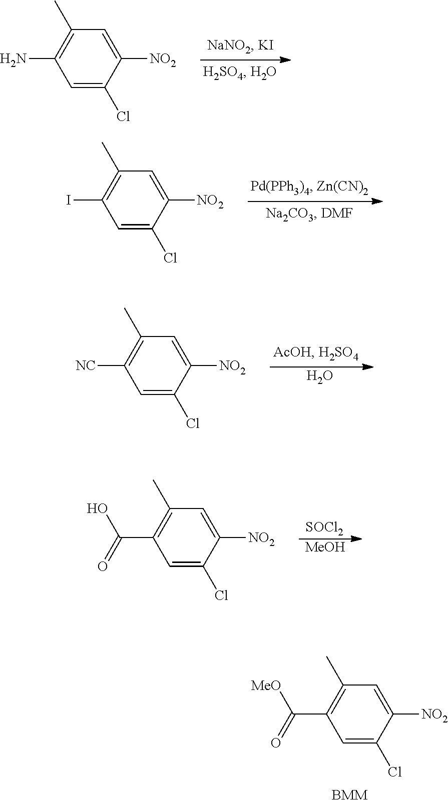

### Step 1—1-Chloro-5-iodo-4-methyl-2-nitrobenzene

To a mixture of 5-chloro-2-methyl-4-nitro-aniline (10 g, 53.6 mmol, CAS #13852-51-2) in H₂SO₄ (3 M, 100 mL) was added dropwise a solution of NaNO₂ (3.70 g, 53.6 mmol) in H₂O (10 mL) over 1 hr at 0° C. Then a solution of KI (10.7 g, 64.3 mmol) in H₂O (10 mL) was added. The reaction mixture was stirred at 0° C. for 1 hr and allowed to warm to 25° C. for 1 hr. On completion, the reaction mixture was diluted with water (500 mL) and extracted with EA (3×200 mL). The organic layer was washed with saturated NaHCO₃ (aq., 2×500 mL) and brine (3×300 mL). The organic layer was dried over Na₂SO₄, filtered and concentrated in vacuo. The residue was purified by silica gel chromatography (PE:EA=1:0 to 100:1) to give the title compound (12.2 g, 76% yield) as yellow solid. ¹H NMR (400 MHz, DMSO-d₆) δ 8.24 (s, 1H), 8.04 (s, 1H), 2.43 (s, 3H).

### Step 2—5-Chloro-2-methyl-4-nitrobenzonitrile

To a mixture of 1-chloro-5-iodo-4-methyl-2-nitro-benzene (11.6 g, 39.2 mmol), Pd(PPh₃)₄ (4.53 g, 3.92 mmol) and Na₂CO₃ (8.31 g, 78.4 mmol) in DMF (80 mL) was added Zn(CN)₂ (2.76 g, 23.5 mmol, 1.49 mL). The reaction mixture was stirred at 50° C. for 24 hrs under N₂. On completion, the reaction mixture was diluted with water (500 mL) and extracted with EA (3×300 mL). The organic layer was washed with brine (3×300 mL), dried over Na₂SO₄, filtered and concentrated in vacuo. The residue was purified by silica gel chromatography (PE:EA=1:0 to 80:1) to give the title compound (6.30 g, 82% yield) as yellow solid. ¹H NMR (400 MHz, CDCl₃) δ 7.82-7.79 (m, 2H), 2.64 (s, 3H).

### Step 3—5-Chloro-2-methyl-4-nitrobenzoic acid

A mixture of 5-chloro-2-methyl-4-nitro-benzonitrile (2.20 g, 11.2 mmol) in a mixed of AcOH (20 mL), H₂O (20 mL) and H₂SO₄ (20 mL) was stirred at 120° C. for 16 hrs. On completion, the reaction mixture was diluted with water (40 mL) and filtered. The filtrate cake was dried in vacuo to give the title compound (2.20 g, 91% yield) as off-white solid. ¹H NMR (400 MHz, CDCl₃) δ 8.23 (s, 1H), 7.76 (s, 1H), 2.71 (s, 3H).

### Step 4—Methyl 5-chloro-2-methyl-4-nitrobenzoate

To a mixture of 5-chloro-2-methyl-4-nitro-benzoic acid (2.20 g, 10.2 mmol) in MeOH (40 mL) was dropwise SOCl₂ (1.82 g, 15.3 mmol, 1.11 mL) at 0° C. The reaction mixture was stirred at 70° C. for 16 hrs under N₂. On completion, the reaction mixture was concentrated in vacuo and the residue was diluted with EA (100 mL) and washed with brine (3×100 mL). The organic layer was dried over Na₂SO₄, filtered and concentrated in vacuo to give the title compound (2.30 g, 98% yield) as off-white solid. ¹H NMR (400 MHz, CDCl₃) δ 8.08 (s, 1H), 7.74 (s, 1H), 3.96 (s, 3H), 2.64 (s, 3H).

### N-(2-((1r,4r)-4-formylcyclohexyl)-6-methoxy-1-oxoisoindolin-5-yl)-6-(trifluoromethyl)picolinamide (Intermediate BMI)

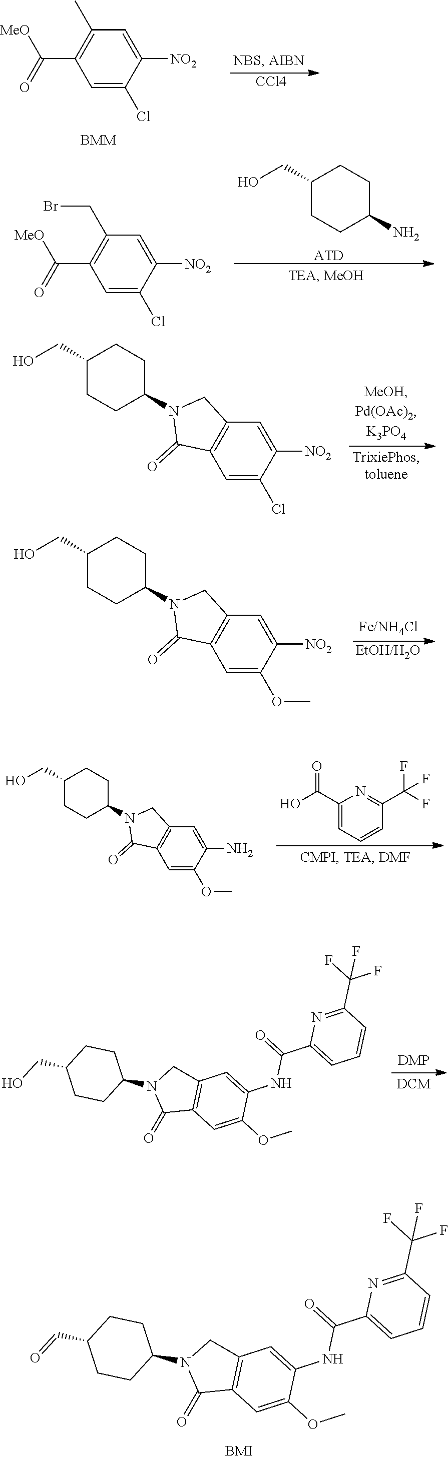

### Step 1—Methyl 2-(bromomethyl)-5-chloro-4-nitrobenzoate

To a solution of methyl 5-chloro-2-methyl-4-nitro-benzoate (3.1 g, 13.5 mmol, Intermediate BMM) in ACN (60 mL) was added NBS (2.88 g, 16.2 mmol) and AIBN (111 mg, 675 umol). The mixture was stirred at 70° C. for 16 hours under N₂. On completion, the reaction mixture was concentrated in vacuo and the residue was diluted with EA (100 mL) and washed with brine (2×100 mL). The organic layer was dried over Na₂SO₄, filtered and concentrated in vacuo to give the title compound (4.0 g, 96% yield) as yellow solid. ¹H NMR (400 MHz, CDCl₃) δ 8.15 (s, 1H), 7.99 (s, 1H), 4.92 (s, 2H), 4.01 (s, 3H).

### Step 2—6-Chloro-2-((1r,4r)-4-(hydroxymethyl)cyclohexyl)-5-nitroisoindolin-1-one

To a solution of methyl 2-(bromomethyl)-5-chloro-4-nitro-benzoate (4.0 g, 13.0 mmol) and (4-aminocyclohexyl)methanol (2.01 g, 15.6 mmol, Intermediate ATD) in MeOH (40 mL) was added TEA (2.62 g, 25.9 mmol, 3.61 mL) and the mixture was stirred at 80° C. for 16 hours under N₂. On completion, the reaction mixture was concentrated in vacuo. The residue was purified by column chromatography (SiO₂, PE:EA=5:1 to 1:2) to give the title compound (2.86 g, 68% yield) as yellow solid. ¹H NMR (400 MHz, CDCl₃) δ 8.01 (s, 1H), 7.91 (s, 1H), 4.44 (s, 2H), 4.25 (tt, J=3.6, 12.1 Hz, 1H), 3.53 (d, J=6.2 Hz, 2H), 2.01-1.93 (m, 4H), 1.62-1.55 (m, 2H), 1.55-1.49 (m, 1H), 1.46 (s, 1H), 1.28-1.17 (m, 2H).

### Step 3—2-((1r,4r)-4-(Hydroxymethyl)cyclohexyl)-6-methoxy-5-nitroisoindolin-1-one

To a solution of 6-chloro-2-[4-(hydroxymethyl)cyclohexyl]-5-nitro-isoindolin-1-one (1.0 g, 3.08 mmol) in MeOH (1.97 g, 61.6 mmol, 2.49 mL) and toluene (10 mL) was added K₃PO₄ (1.31 g, 6.16 mmol), Pd(OAc)₂ (69.1 mg, 308 umol) and ditert-butyl-[1-(1-naphthyl)-2-naphthyl]phosphane (245 mg, 616 umol), and the mixture was stirred at 80° C. for 16 hours under N₂. On completion, the reaction mixture was concentrated in vacuo and purified by column chromatography (SiO₂, PE/EA=10/1 to 1/10) to give the title compound (500 mg, 51% yield) as yellow solid. ¹H NMR (400 MHz, CDCl₃) δ 7.86 (s, 1H), 7.55 (s, 1H), 4.37 (s, 2H), 4.31-4.21 (m, 1H), 4.02 (s, 3H), 3.53 (d, J=1.8 Hz, 2H), 1.97 (d, J=11.0 Hz, 4H), 1.61-1.49 (m, 3H), 1.44 (s, 1H), 1.29-1.17 (m, 2H).

### Step 4—5-Amino-2-((1r,4r)-4-(hydroxymethyl)cyclohexyl)-6-methoxyisoindolin-1-one

To a solution of 2-[4-(hydroxymethyl)cyclohexyl]-6-methoxy-5-nitro-isoindolin-1-one (500 mg, 1.56 mmol) in the EtOH (6.0 mL) and H₂O (2.0 mL) was added Fe (436 mg, 7.80 mmol) and NH₄Cl (417 mg, 7.80 mmol). The mixture was stirred at 80° C. for 4 hours. On completion, the reaction mixture was filtered and concentrated in vacuo. The residue was diluted with DCM (100 mL) and MeOH (1.0 mL, then the mixture was washed with water (100 mL). The organic layer was dried over Na₂SO₄, filtered and concentrated in vacuo to give the title compound (400 mg, 88% yield) as yellow solid. ¹H NMR (400 MHz, DMSO-d₆) δ 6.98 (s, 1H), 6.72 (s, 1H), 5.36 (s, 2H), 4.43 (s, 1H), 4.19 (s, 2H), 3.90 (t, J=11.8 Hz, 1H), 3.81 (s, 3H), 3.24 (t, J=5.2 Hz, 2H), 1.82 (d, J=12.4 Hz, 2H), 1.71 (d, J=11.2 Hz, 2H), 1.50 (q, J=11.8 Hz, 2H), 1.38-1.29 (m, 1H), 1.03 (q, J=12.0 Hz, 2H).

### Step 5—N-(2-((1r,4r)-4-(hydroxymethyl)cyclohexyl)-6-methoxy-1-oxoisoindolin-5-yl)-6-(trifluoromethyl)picolinamide

To a solution of 5-amino-2-[4-(hydroxymethyl)cyclohexyl]-6-methoxy-isoindolin-1-one (100 mg, 344 umol) and 6-(trifluoromethyl)pyridine-2-carboxylic acid (65.8 mg, 344 umol) in the DMF (2 mL) was added CMPI (106 mg, 413 umol) and DIEA (133 mg, 1.03 mmol, 180 uL). The mixture was stirred at 25° C. for 1 hr. On completion, the reaction mixture was diluted with EA (20 mL) and washed with water (3×20 mL). The organic layer was dried over Na₂SO₄, filtered and concentrated in vacuo to give the title compound (155 mg, 97% yield) as yellow solid. ¹H NMR (400 MHz, CDCl₃) δ 10.76 (s, 1H), 8.74 (s, 1H), 8.50 (d, J=7.8 Hz, 1H), 8.15 (t, J=7.8 Hz, 1H), 7.90 (d, J=7.6 Hz, 1H), 7.41 (s, 1H), 4.34 (s, 2H), 4.25 (t, J=11.6 Hz, 1H), 4.05 (s, 3H), 3.53 (d, J=6.4 Hz, 2H), 1.97 (d, J=11.0 Hz, 4H), 1.62-1.54 (m, 3H), 1.29-1.24 (m, 2H).

### Step 6—N-(2-((1r,4r)-4-formylcyclohexyl)-6-methoxy-1-oxoisoindolin-5-yl)-6-(trifluoromethyl) picolinamide

To a solution of N-[2-[4-(hydroxymethyl)cyclohexyl]-6-methoxy-1-oxo-isoindolin-5-yl]-6-(trifluoromethyl)pyridine-2-carboxamide (155 mg, 334 umol) in the DCM (3 mL) was added DMP (170 mg, 401 umol) and the mixture was stirred at 25° C. for 1 hr. On completion, the reaction mixture was quenched by Na₂S₂O₃ (aq, 5 mL) and NaHCO₃ (aq, 5 mL), then the mixture was extracted with DCM (2×20 mL). The organic layer was dried over Na₂SO₄, filtered and concentrated in vacuo and purified by column chromatography (SiO2, PE/EA=10/1 to 1/1) to give the title compound (150 mg, 87% yield) as yellow solid. LC-MS (ESI⁺) m/z 462.2 (M+H)⁺.

### N-[2-(4-formylcyclohexyl)-6-[(1S,4S)-2-oxa-5-azabicyclo[2.2.1]heptan-5-yl]-1-oxo-isoindolin-5-yl]-6-(trifluoromethyl)pyridine-2-carboxamide (Intermediate BQE)

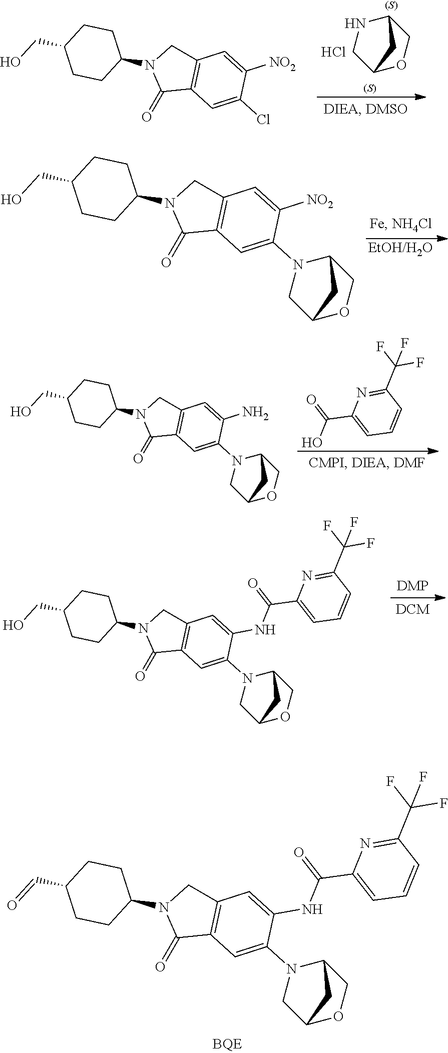

### Step 1—2-[4-(Hydroxymethyl)cyclohexyl]-5-nitro-6-[(1S,4S)-2-oxa-5-azabicyclo[2.2.1]heptan-5-yl]isoindolin-1-one

To a solution of 6-chloro-2-[4-(hydroxymethyl)cyclohexyl]-5-nitro-isoindolin-1-one (220 mg, 677 umol, synthesized via Steps 1-2 of Intermediate BMI) and (1S,4S)-2-oxa-5-azabicyclo[2.2.1]heptane (137.78 mg, 1.02 mmol, HCl salt, CAS #31560-06-2) in DMSO (3 mL) was added DIEA (175 mg, 1.35 mmol) at 25° C. Then the reaction mixture was stirred at 80° C. for 36 hrs. On completion, the reaction mixture was concentrated in vacuo. The residue was purified by prep-HPLC (column: Phenomenex luna C18 150*25 mm*10 um; mobile phase: [water (0.225% FA)-ACN]; B %: 22%-52%, 10 min) to give the title compound (45.0 mg, 17% yield) as a yellow solid. ¹H NMR (400 MHz, DMSO-d₆) δ 7.97 (s, 1H), 7.30 (s, 1H), 4.39 (s, 2H), 4.02-3.92 (m, 1H), 3.83-3.79 (m, 1H), 3.77-3.74 (m, 1H), 3.45 (d, J=10.0 Hz, 2H), 3.24 (d, J=5.6 Hz, 2H), 2.40 (d, J=10.0 Hz, 1H), 1.99-1.88 (m, 3H), 1.87-1.72 (m, 5H), 1.61-1.49 (m, 2H), 1.42-1.31 (m, 1H), 1.12-1.00 (m, 2H); LC-MS (ESI⁺) m/z 388.1 (M+H)⁺.

### Step 2—5-Amino-2-[4-(hydroxymethyl)cyclohexyl]-6-[(1S,4S)-2-oxa-5-azabicyclo[2.2.1]heptan-5-yl]isoindolin-1-one

To a solution of 2-[4-(hydroxymethyl)cyclohexyl]-5-nitro-6-[(1S,4S)-2-oxa-5-azabicyclo[2.2.1]heptan-5-yl]isoindolin-1-one (45.0 mg, 116 umol) in THF (0.1 mL), EtOH (0.5 mL) and H₂O (0.5 mL) was added NH₄Cl (62.1 mg, 1.16 mmol) and Fe (32.4 mg, 580 umol) at 70° C. The reaction mixture was then stirred at 80° C. for 2 hrs. On completion, the reaction mixture was filtered with celite and the filtrate was concentrated in vacuo to give a residue. The residue was diluted with H₂O (30 mL) and extracted with EA (3×10 mL). The combined organic layers were washed with brine 30 mL (2×15 mL), dried over by Na₂SO₄, filtered and concentrated in vacuo to give the title compound (35.0 mg, 84% yield) as a yellow solid. LC-MS (ESI⁺) m/z 358.1 (M+H)⁺.

### Step 3—N-[2-[4-(hydroxymethyl)cyclohexyl]-6-[(1S,4S)-2-oxa-5-azabicyclo[2.2.1]heptan-5-yl]-1-oxo-isoindolin-5-yl]-6-(trifluoromethyl)pyridine-2-carboxamide

To a solution of 5-amino-2-[4-(hydroxymethyl)cyclohexyl]-6-[(1S,4S)-2-oxa-5-azabicyclo[2.2.1]heptan-5-yl]isoindolin-1-one (35.0 mg, 97.9 umol) and 6-(trifluoromethyl)pyridine-2-carboxylic acid (18.71 mg, 97.9 umol, CAS #131747-42-7) in DMF (3 mL) was added DIEA (25.3 mg, 195 umol) and CMPI (40.0 mg, 156 umol) at 25° C. The reaction mixture was stirred at 25° C. for 1 hr. On completion, the reaction mixture was concentrated in vacuo. The residue was purified by reverse phase (0.1% FA condition) to give the title compound (17.0 mg, 32% yield) as a white solid. LC-MS (ESI⁺) m/z 531.4 (M+H)⁺.

### Step 4—N-[2-(4-formylcyclohexyl)-6-[(1S,4S)-2-oxa-5-azabicyclo[2.2.1]heptan-5-yl]-1-oxo-isoindolin-5-yl]-6-(trifluoromethyl)pyridine-2-carboxamide

To a solution of N-[2-[4-(hydroxymethyl)cyclohexyl]-6-[(1S,4S)-2-oxa-5-azabicyclo[2.2.1]heptan-5-yl]-1-oxo-isoindolin-5-yl]-6-(trifluoromethyl)pyridine-2-carboxamide (17.0 mg, 32.0 umol) in DCM (1 mL) was added DMP (21.7 mg, 51.2 umol) at 25° C. The reaction mixture was then stirred at 25° C. for 1 hr. On completion, the reaction mixture was quenched by addition 1 mL sat. aq. Na₂S₂O₃ and 1 mL NaHCO₃ and then diluted with 15 mL H₂O. The mixture was extracted with DCM (3×5 mL) and the combined organic layers were washed with brine 10 mL (2×5 mL), dried over by Na₂SO₄, filtered and concentrated in vacuo to give the title compound (8.00 mg, 47% yield) as a white solid. LC-MS (ESI⁺) m/z 529.1 (M+H)⁺.

### 3-(3-Methyl-2-oxo-4-piperazin-1-yl-benzimidazol-1-yl)piperidine-2,6-dione (Intermediate BAI)

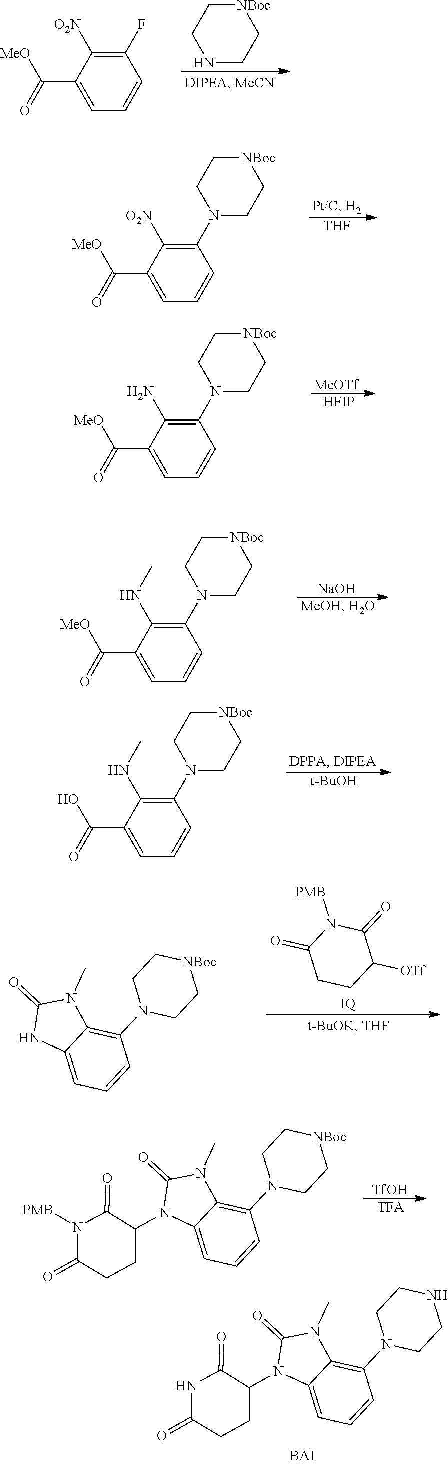

### Step 1—Tert-butyl 4-(3-methoxycarbonyl-2-nitro-phenyl)piperazine-1-carboxylate

To a solution of methyl 3-fluoro-2-nitro-benzoate (10.0 g, 50.2 mmol, CAS #1214353-57-7) and tert-butyl piperazine-1-carboxylate (11.2 g, 60.3 mmol, CAS #143238-38-4) in ACN (100 mL) was added DIPEA (19.5 g, 151 mmol). The reaction mixture was stirred at 50° C. for 12 hrs. On completion, the mixture was concentrated in vacuo. The residue was dissolved in water (200 mL), then extracted with EA (2×200 mL). The organic layer was washed with brine (2×100 mL), dried with Na₂SO₄, filtered and the filtrate was concentrated in vacuo to give the title compound (18.3 g, 100% yield) as yellow oil. ¹H NMR (400 MHz, DMSO-d₆) δ 7.86 (dd, J=1.2, 8.0 Hz, 1H), 7.82-7.79 (m, 1H), 7.74-7.68 (m, 1H), 3.83 (s, 3H), 3.40-3.35 (m, 4H), 2.88-2.84 (m, 4H), 1.41 (s, 9H).

### Step 2—Tert-butyl 4-(2-amino-3-methoxycarbonyl-phenyl)piperazine-1-carboxylate

To a solution of tert-butyl 4-(3-methoxycarbonyl-2-nitro-phenyl)piperazine-1-carboxylate (17.0 g, 46.5 mmol) in THF (15 mL) was added Pd/C (2.00 g, 10 wt %). The reaction mixture was stirred at 20° C. for 12 hrs under H₂ (15 Psi) atmosphere. On completion, the mixture was filtered and the filtrate was concentrated in vacuo to give the title compound (15.2 g, 97% yield) as a yellow solid. ¹H NMR (400 MHz, CDCl₃) δ 7.67 (dd, J=1.2, 8.0 Hz, 1H), 7.10 (dd, J=1.2, 7.6 Hz, 1H), 6.61 (t, J=7.6 Hz, 1H), 6.24 (br s, 2H), 4.28-3.95 (m, 2H), 3.87 (s, 3H), 3.16-2.84 (m, 4H), 2.80-2.55 (m, 2H), 1.49 (s, 9H).

### Step 3—Tert-butyl 4-[3-methoxycarbonyl-2-(methylamino)phenyl]piperazine-1-carboxylate

To a solution of tert-butyl 4-(2-amino-3-methoxycarbonyl-phenyl)piperazine-1-carboxylate (15.0 g, 44.7 mmol) in 1,1,1,3,3,3-hexafluoropropan-2-ol (40 mL) was added methyl trifluoromethanesulfonate (9.54 g, 58.1 mmol) at 0° C. The reaction mixture was stirred at 0° C. for 0.5 hr. On completion, the mixture was diluted with water (200 mL), then extracted with EA (2×200 mL). The organic layer was washed with brine (2×200 mL), dried with Na₂SO₄, filtered and the filtrate was concentrated in vacuo to give the title compound (15.0 g, 96% yield) as a yellow solid. ¹H NMR (400 MHz, DMSO-d₆) δ 8.16-7.90 (m, 1H), 7.46 (dd, J=1.2, 8.0 Hz, 1H), 7.23 (d, J=7.6 Hz, 1H), 6.77 (t, J=7.6 Hz, 1H), 3.80 (s, 3H), 3.55-3.45 (m, 4H), 2.87 (s, 3H), 2.80-2.74 (m, 4H), 1.42 (s, 9H).

### Step 4—3-(4-Tert-butoxycarbonylpiperazin-1-yl)-2-(methylamino)benzoic acid

To a solution of tert-butyl 4-[3-methoxycarbonyl-2-(methylamino)phenyl]piperazine-1-carboxylate (14.0 g, 40.1 mmol) in a mixed solvent of H₂O (20 mL) and MeOH (140 mL) was added NaOH (4.81 g, 120 mmol). The reaction mixture was stirred at 70° C. for 12 hrs. On completion, the mixture was concentrated in vacuo. The residue was diluted with water (200 mL), and extracted with EA (100 mL). The organic layer was discarded. The aqueous phase was acidified with HCl (1N) to pH=3-5, and extracted with EA (2×100 mL). The organic layer was washed with brine (200 mL), dried with Na₂SO₄, filtered and the filtrate was concentrated in vacuo. The residue was triturated with MeOH/H₂O (1:10, 100 mL) and filtered. The filter cake was dried in vacuo to give the title compound (9.60 g, 71% yield) as a white solid. LC-MS (ESI⁺) m/z 336.1 (M+H)⁺.

### Step 5—Tert-butyl 4-(3-methyl-2-oxo-1H-benzimidazol-4-yl)piperazine-1-carboxylate

To a solution of 3-(4-tert-butoxycarbonylpiperazin-1-yl)-2-(methylamino)benzoic acid (9.60 g, 28.6 mmol) and DIPEA (11.1 g, 85.9 mmol) in t-BuOH (200 mL) was added DPPA (7.88 g, 28.6 mmol). The reaction mixture was stirred at 85° C. for 12 hrs. On completion, the mixture was concentrated in vacuo. The residue was diluted with water (200 mL), and extracted with EA (2×200 mL). The organic layer was washed with brine (200 mL) and concentrated in vacuo. The residue was purified by reverse phase (0.1% FA condition) to give the title compound (3.35 g, 35% yield) as a white solid. ¹H NMR (400 MHz, DMSO-d₆) δ 10.84 (s, 1H), 6.94-6.87 (m, 1H), 6.85-6.79 (m, 1H), 6.75 (dd, J=1.2, 7.6 Hz, 1H), 4.06-3.80 (m, 2H), 3.55 (s, 3H), 3.20-2.87 (m, 4H), 2.76-2.56 (m, 2H), 1.42 (s, 9H).

### Step 6—Tert-butyl 4-[1-[1-[(4-methoxyphenyl)methyl]-2,6-dioxo-3-piperidyl]-3-methyl-2-oxo-benzimidazol-4-yl]piperazine-1-carboxylate

To a solution of tert-butyl 4-(3-methyl-2-oxo-1H-benzimidazol-4-yl)piperazine-1-carboxylate (3.30 g, 9.93 mmol) in THF (50 mL) was added t-BuOK (1.67 g, 14.9 mmol) at 0° C. 1 hr later, and a solution of [1-[(4-methoxyphenyl)methyl]-2,6-dioxo-3-piperidyl]trifluoromethanesulfonate (4.54 g, 11.9 mmol, Intermediate IQ) in THF (20 mL) was added. The reaction mixture was stirred at 0° C. for 3 hrs. On completion, the mixture was acidified with FA to pH=3-5, diluted with water (300 mL), then extracted with EA (2×300 mL). The organic layer was washed with brine (200 mL) and concentrated in vacuo. The residue was purified by reverse phase flash (0.1% FA condition) to give the title compound (3.90 g, 70% yield) as a white solid. LC-MS (ESI⁺) m/z 564.3 (M+H)⁺.

### Step 7—3-(3-Methyl-2-oxo-4-piperazin-1-yl-benzimidazol-1-yl)piperidine-2,6-dione

To a solution of tert-butyl 4-[1-[1-[(4-methoxyphenyl)methyl]-2,6-dioxo-3-piperidyl]-3-methyl-2-oxo-benzimidazol-4-yl]piperazine-1-carboxylate (3.90 g, 6.92 mmol) in TFA (40 mL) was added TfOH (5 mL). The reaction mixture was stirred at 65° C. for 12 hrs. On completion, the mixture was concentrated in vacuo. The residue was purified by reverse phase flash (0.10% FA condition) to give the title compound (1.70 g, 63% yield, FA salt) as a blue solid. ¹H NMR (400 MHz, DMSO-d₆) δ 11.09 (s, 1H), 7.06-6.99 (m, 1H), 6.96-6.92 (m, 2H), 5.36 (dd, J=5.2, 12.4 Hz, 1H), 3.63 (s, 3H), 3.35-3.25 (m, 4H), 3.16-2.97 (m, 4H), 2.91-2.82 (m, 1H), 2.76-2.57 (m, 2H), 2.05-1.93 (m, 1H).

### N-[2-(4-formylcyclohexyl)-6-morpholino-1-oxo-isoindolin-5-yl]-6-(trifluoromethyl)pyridine-2-carboxamide (Intermediate BOA)

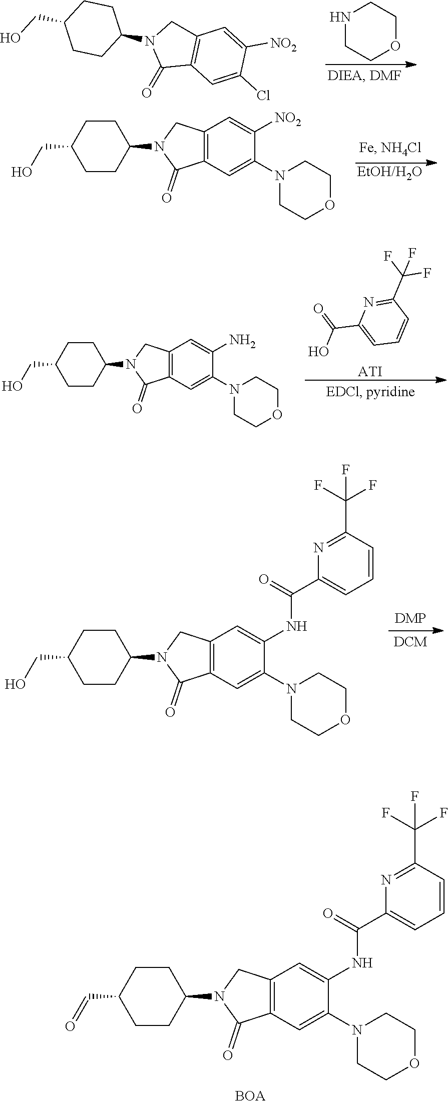

### Step 1—2-[4-(Hydroxymethyl)cyclohexyl]-6-morpholino-5-nitro-isoindolin-1-one

To a solution of 6-chloro-2-[4-(hydroxymethyl)cyclohexyl]-5-nitro-isoindolin-1-one (300 mg, 923 umol, synthesized via Steps 1-2 of Intermediate BMI) and morpholine (104 mg, 1.20 mmol) in DMSO (4 mL) was added DIEA (238 mg, 1.85 mmol) at 25° C. The reaction mixture was stirred at 80° C. for 12 hours. On completion, the reaction mixture was concentrated in vacuo. The residue was purified by reverse phase (0.1% FA condition) to give the title compound (240 mg, 69% yield) as a yellow solid. LC-MS (ESI⁺) m/z 375.9 (M+H)⁺.

### Step 2—5-Amino-2-[4-(hydroxymethyl)cyclohexyl]-6-morpholino-isoindolin-1-one

To a solution of 2-[4-(hydroxymethyl)cyclohexyl]-6-morpholino-5-nitro-isoindolin-1-one (140 mg, 372 umol) in H₂O (2.5 mL), EtOH (2.5 mL) and THF (0.5 mL) was added NH₄Cl (199 mg, 3.73 mmol) at 25° C. Then Fe (104 mg, 1.86 mmol) was added to the above mixture at 70° C., and the reaction mixture was stirred at 80° C. for 2 hours. On completion, the reaction mixture was filtered with celite and the filtrate was concentrated in vacuo. The residue was diluted with H₂O (30 mL) and extracted with EA (3×10 mL). The combined organic layers were washed with brine (2×15 mL), dried over Na₂SO₄, filtered and concentrated in vacuo. The residue was purified by reverse phase (0.10% FA condition) to give the title compound (30.0 mg, 23% yield) as a white solid. LC-MS (ESI⁺) m/z 346.2 (M+H)⁺.

### Step 3—N-[2-[4-(hydroxymethyl)cyclohexyl]-6-morpholino-1-oxo-isoindolin-5-yl]-6-(trifluoro methy)pyridine-2-carboxamide

To a solution of 5-amino-2-[4-(hydroxymethyl)cyclohexyl]-6-morpholino-isoindolin-1-one (30.0 mg, 86.8 umol) and 6-(trifluoromethyl)pyridine-2-carboxylic acid (19.9 mg, 104 umol, Intermediate ATI) in pyridine (0.6 mL) was added EDCI (24.9 mg, 130 umol) and the reaction mixture was stirred at 25° C. for 12 hours. On completion, the reaction mixture was concentrated in vacuo. The residue was purified by reverse phase (0.1% FA condition) to give the title compound (20.0 mg, 44% yield) as a white solid. LC-MS (ESI⁺) m/z 519.2 (M+H)⁺.

### Step 4—N-[2-(4-formylcyclohexyl)-6-morpholino-1-oxo-isoindolin-5-yl]-6-(trifluoromethyl)pyridine-2-carboxamide

To a solution of N-[2-[4-(hydroxymethyl)cyclohexyl]-6-morpholino-1-oxo-isoindolin-5-yl]-6-(trifluoromethyl)pyridine-2-carboxamide (15.0 mg, 28.9 umol) in DCM (1 mL) was added DMP (15.9 mg, 37.6 umol) and the reaction mixture was stirred at 25° C. for 1 hour. On completion, the reaction mixture was quenched with sat. aq. Na₂S₂O₃ (1 mL) and sat. aq. NaHCO₃ (1 mL) and diluted with H₂O (15 mL) and were extracted with DCM (3×5 mL). The combined organic layers were washed with brine 10 mL (2×5 mL), dried over Na₂SO₄, filtered and concentrated in vacuo to give the title compound (14.0 mg, 93% yield) as a white solid. LC-MS (ESI⁺) m/z 517.2 (M+H)⁺.

### N-[2-(4-formylcyclohexyl)-6-[(1R,4R)-2-oxa-5-azabicyclo[2.2.1]heptan-5-yl]-1-oxo-isoindolin-5-yl]-6-(trifluoromethyl)pyridine-2-carboxamide (Intermediate BQF)

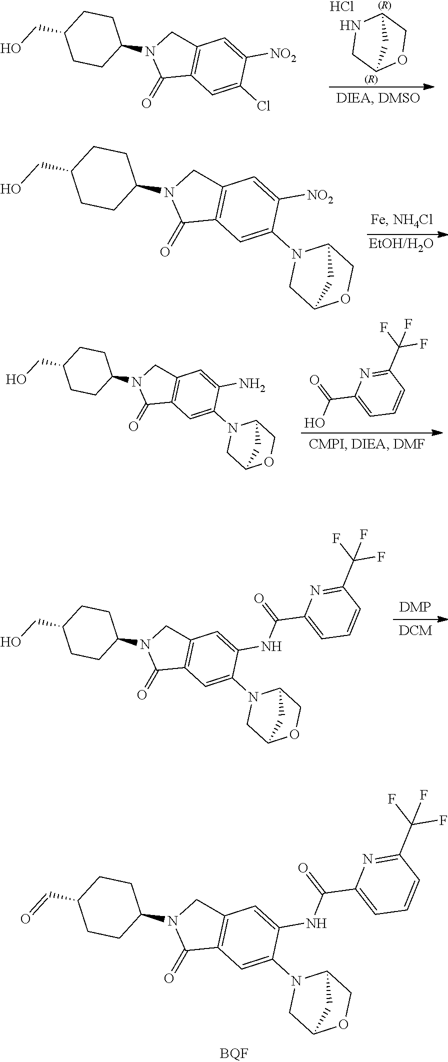

### Step 1—2-[4-(Hydroxymethyl)cyclohexyl]-5-nitro-6-[(1R,4R)-2-oxa-5-azabicyclo[2.2.1]heptan-5-yl]isoindolin-1-one

To a solution of 6-chloro-2-[4-(hydroxymethyl)cyclohexyl]-5-nitro-isoindolin-1-one (220 mg, 677 umol, synthesized via Steps 1-2 of Intermediate BMI) and (1R,4R)-2-oxa-5-azabicyclo[2.2.1]heptane (183 mg, 1.35 mmol, HCl salt, CAS #279-33-4) in DMSO (4 mL) was added DIEA (262 mg, 2.03 mmol) at 25° C. The reaction mixture was then stirred at 80° C. for 36 hrs. On completion, the reaction mixture was concentrated in vacuo. The residue was purified by reverse phase (0.1% FA condition) to give the title compound (55.0 mg, 20% yield) as a yellow solid. LC-MS (ESI⁺) m/z 388.4 (M+H)⁺.

### Step 2—5-Amino-2-[4-(hydroxymethyl)cyclohexyl]-6-[(1R,4R)-2-oxa-5-azabicyclo[2.2.1]heptan-5-yl]isoindolin-1-one

To a solution of 2-[4-(hydroxymethyl)cyclohexyl]-5-nitro-6-[(1R,4R)-2-oxa-5-azabicyclo[2.2.1]heptan-5-yl]isoindolin-1-one (55.0 mg, 141 umol) in EtOH (1 mL), THF (0.2 mL) and H₂O (1 mL) was added NH₄Cl (75.9 mg, 1.42 mmol) and Fe (39.6 mg, 709 umol) at 70° C. Then the reaction mixture was stirred at 80° C. for 1 hr. On completion, the reaction mixture was filtered with celite and the filtrate was concentrated in vacuo to give a residue. The residue was diluted with 30 mL H₂O and extracted with EA (3×10 mL). The combined organic layers were washed with brine (2×15 mL), dried over by Na2SO4, filtered and concentrated in vacuo to give the title compound (35.0 mg, 68% yield) as a yellow solid. LC-MS (ESI⁺) m/z 357.9 (M+H)⁺.

### Step 3—N-[2-[4-(hydroxymethyl)cyclohexyl]-6-[(1R,4R)-2-oxa-5-azabicyclo[2.2.1]heptan-5-yl]-1-oxo-isoindolin-5-yl]-6-(trifluoromethyl)pyridine-2-carboxamide

To a solution of 5-amino-2-[4-(hydroxymethyl)cyclohexyl]-6-[(1R,4R)-2-oxa-5-azabicyclo[2.2.1]heptan-5-yl]isoindolin-1-one (35.0 mg, 97.9 umol) and 6-(trifluoromethyl)pyridine-2-carboxylic acid (18.7 mg, 97.9 umol, CAS #131747-42-7) in DMF (1 mL) was added DIEA (25.3 mg, 195 umol) and CMPI (40.0 mg, 156 umol). The mixture was stirred at 25° C. for 1 hr. On completion, the reaction mixture was concentrated in vacuo. The residue was purified by reverse phase (0.1% FA condition) to give the title compound (26.0 mg, 50% yield) as a white solid. LC-MS (ESI⁺) m/z 531.4 (M+H)⁺.

### Step 4—N-[2-(4-formylcyclohexyl)-6-[(1R,4R)-2-oxa-5-azabicyclo[2.2.1]heptan-5-yl]-1-oxo-isoindolin-5-yl]-6-(trifluoromethyl)pyridine-2-carboxamide

To a solution of N-[2-[4-(hydroxymethyl)cyclohexyl]-6-[(1R,4R)-2-oxa-5-azabicyclo[2.2.1]heptan-5-yl]-1-oxo-isoindolin-5-yl]-6-(trifluoromethyl)pyridine-2-carboxamide (26.0 mg, 49.0 umol) in DCM (1 mL) was added DMP (33.2 mg, 78.4 umol) at 25° C. and the reaction mixture was stirred at 25° C. for 1 hr. On completion, the reaction mixture was quenched by addition sat.aq. Na₂S₂O₃ (1 mL) and NaHCO₃ (1 mL) and then diluted with H₂O 15 mL, then the mixture was extracted with DCM (3×5 mL). The combined organic layers were washed with brine 10 mL (2×5 mL), dried over by Na₂SO₄, filtered and concentrated in vacuo to give the title compound (20.0 mg, 77% yield) as a white solid. LC-MS (ESI⁺) m/z 529.2 (M+H)⁺.

### N-(6-fluoro-2-((1R,3R)-3-formylcyclobutyl)-2H-indazol-5-yl)-6-(trifluoromethyl)picolinamide (Intermediate BQH)

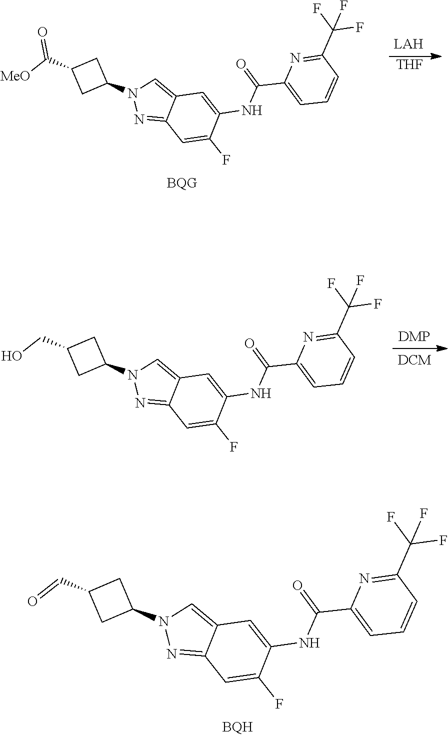

### Step 1—N-(6-Fluoro-2-((1R,3R)-3-(hydroxymethyl)cyclobutyl)-2H-indazol-5-yl)-6-(trifluoromethyl) picolinamide

To a solution of methyl 3-[6-fluoro-5-[[6-(trifluoromethyl)pyridine-2-carbonyl]amino]indazol-2-yl]cyclobutanecarboxylate (170 mg, 389 umol, Intermediate BQG) in THF (4 mL) was added LiAlH₄ (29.5 mg, 779 umol). Then the mixture was stirred at 0° C. for 1 hour. The reaction mixture was quenched by addition 15% seignette salt (0.1 mL) at 0° C., and then diluted with THF (10 mL), filtered and concentrated under reduced pressure to give the title compound (100 mg, 52% yield) as an off-white solid. LC-MS (ESI⁺) m/z 408.9 (M+H)⁺. ¹H NMR (400 MHz, CDCl₃) δ 10.25 (d, J=3.2 Hz, 1H), 8.81 (d, J=7.8 Hz, 1H), 8.51 (d, J=7.8 Hz, 1H), 8.15 (t, J=7.8 Hz, 1H), 7.98 (s, 1H), 7.90 (dd, J=0.8, 7.8 Hz, 1H), 7.47 (d, J=11.6 Hz, 1H), 5.14 (t, J=7.8 Hz, 1H), 3.84 (d, J=6.2 Hz, 2H), 2.98-2.84 (m, 2H), 2.78-2.64 (m, 1H), 2.60-2.47 (m, 2H).

### Step 2—N-(6-fluoro-2-((1R,3R)-3-formylcyclobutyl)-2H-indazol-5-yl)-6-(trifluoromethyl)picolinamide

To a solution of N-[6-fluoro-2-[3-(hydroxymethyl)cyclobutyl]indazol-5-yl]-6-(trifluoromethyl) pyridine-2-carboxamide (159 mg, 389 umol) in DCM (2 mL) was added DMP (247 mg, 584 umol). The mixture was stirred at 20° C. for 1 hour. On completion, the reaction mixture was quenched by addition sat. NaHCO₃ (10 mL) and sat. Na₂SO₃ (10 mL) at 0° C., and then diluted with H₂O (10 mL) and extracted with EA (3×30 mL). The combined organic layers were washed with brine (20 mL), dried over Na₂SO₄, filtered and concentrated under reduced pressure to give the title compound (100 mg, 46% yield) as an off-white solid. LC-MS (ESI⁺) m/z 407.3 (M+H)⁺.

### N-[2-[3-(hydroxymethyl)cyclobutyl]-6-methoxy-indazol-5-yl]-6-(trifluoromethyl)pyridine-2-carboxamide (Intermediate BQI)

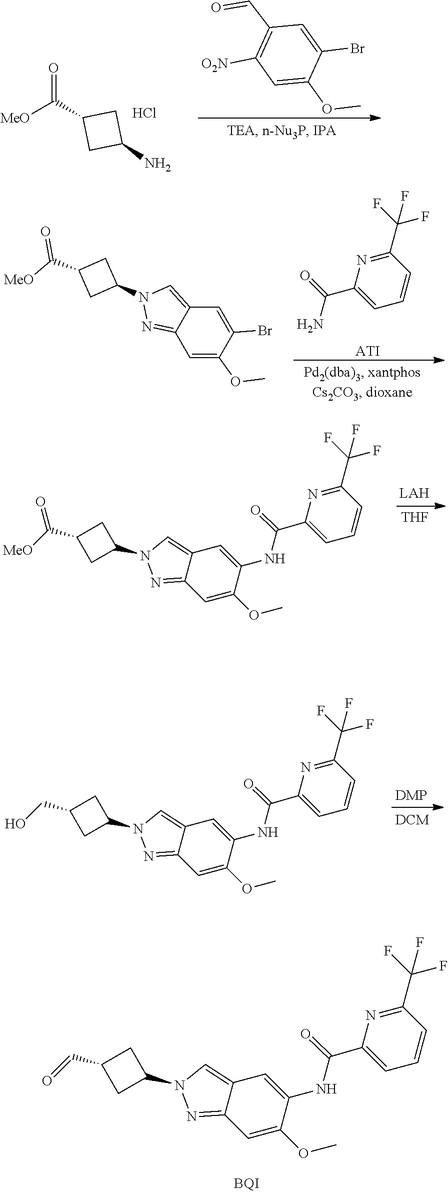

### Step 1—Methyl 3-(5-bromo-6-methoxy-indazol-2-yl)cyclobutanecarboxylate

To a solution of methyl 3-aminocyclobutanecarboxylate (3 g, 18.11 mmol, HCl salt, CAS #74316-29-3) in IPA (60 mL) was added Et₃N (1.83 g, 18.1 mmol, 2.52 mL) and 5-bromo-4-methoxy-2-nitro-benzaldehyde (5.18 g, 19.9 mmol, synthesized via Steps 1-2 of Intermediate ATE) and the mixture was stirred at 80° C. for 4 hrs. After the reaction was cooled to rt, tributylphosphane (10.9 g, 54.3 mmol, 13.41 mL) was added to the mixture and the mixture was stirred at 80° C. for 4 hrs. On completion, the mixture was concentrated in vacuo. The residue was purified by column chromatography (SiO₂, PE:EA 5:1) to give the title compound (900 mg, 15% yield) as yellow solid. ¹H NMR (400 MHz, DMSO-d₆) δ 8.34 (d, J=0.8 Hz, 1H), 7.97 (s, 1H), 7.14 (s, 1H), 5.32-5.22 (m, 1H), 3.87 (s, 3H), 3.69 (s, 3H), 3.33-3.27 (m, 1H), 2.94-2.84 (m, 2H), 2.77-2.69 (m, 2H).

### Step 2—Methyl 3-[6-methoxy-5-[[6-(trifluoromethyl)pyridine-2-carbonyl]amino]indazol-2-yl]cyclobutanecarboxylate

To a solution of methyl 3-(5-bromo-6-methoxy-indazol-2-yl)cyclobutanecarboxylate (600 mg, 1.77 mmol) and 6-(trifluoromethyl)pyridine-2-carboxamide (403 mg, 2.12 mmol, Intermediate ATI) in DMA (20 mL) was added BrettPhos Pd G₃ (160 mg, 176 umol), Cs₂CO₃ (1.15 g, 3.54 mmol) and 4A molecular sieves (100 mg). The mixture was stirred at 90° C. for 6 hrs. On completion, the mixture was filtered with celite. The crude product was purified by reversed-phase HPLC (0.1% FA condition) to give the title compound (500 mg, 63% yield) as yellow solid. ¹H NMR (400 MHz, DMSO-d₆) δ 10.51 (s, 1H), 8.68 (s, 1H), 8.48-8.36 (m, 3H), 8.22 (d, J=7.6 Hz, 1H), 7.20 (s, 1H), 5.25 (q, J=7.6 Hz, 1H), 3.99 (s, 3H), 3.70 (s, 3H), 3.31-3.27 (m, 1H), 2.97-2.85 (m, 2H), 2.81-2.69 (m, 2H); LC-MS (ESI⁺) m/z 449.3 (M+H)⁺.

### Step 3—N-[2-[3-(hydroxymethyl)cyclobutyl]-6-methoxy-indazol-5-yl]-6-(trifluoromethyl)pyridine-2-carboxamide

To a solution of methyl 3-[6-methoxy-5-[[6-(trifluoromethyl) pyridine-2-carbonyl]amino]indazol-2-yl]cyclobutanecarboxylate (150 mg, 334 umol) in THF (1 mL) was added LiAlH₄ (25.4 mg, 669 umol) and the mixture was stirred at 0° C. for 1 hr under N₂. On completion, water (0.5 mL) was added to the mixture at 0° C., then 15% NaOH. aq (0.5 mL) was added, and finally water (1.5 mL) was added. The mixture was dried over Na₂SO₄, filtered and concentrated under reduced pressure to give the title compound (130 mg, 92% yield) as yellow solid. ¹H NMR (400 MHz, DMSO-d₆) δ 10.50 (s, 1H), 8.67 (s, 1H), 8.49-8.32 (m, 3H), 8.21 (br d, J=7.6 Hz, 1H), 7.18 (s, 1H), 5.13 (br t, J=8.0 Hz, 1H), 4.98-4.62 (m, 2H), 3.98 (s, 3H), 3.58 (br s, 1H), 2.71-2.62 (m, 2H), 2.38-2.30 (m, 2H); LC-MS (ESI⁺) m/z 421.2 (M+H)⁺.

### Step 4—N-[2-[3-(hydroxymethyl)cyclobutyl]-6-methoxy-indazol-5-yl]-6-(trifluoromethyl)pyridine-2-carboxamide

To a solution of N-[2-[3-(hydroxymethyl)cyclobutyl]-6-methoxy-indazol-5-yl]-6-(trifluoromethyl)pyridine-2-carboxamide (65.0 mg, 154 umol) in DCM (10 mL) was added DMP (98.3 mg, 231 umol) and the mixture was stirred at 25° C. for 2 hrs. On completion, to the mixture was added sat. NaHCO₃. aq (10 ml) and sat. Na₂S₂O₃. aq (10 ml), and the mixture was extracted with DCM 150 mL (3×50 mL). The combined organic layers were washed with brine 100 mL, dried over Na₂SO₄, filtered and concentrated under reduced pressure to give the title compound (64.0 mg, 99% yield) as yellow solid. LC-MS (ESI⁺) m/z 421.2 (M+H)⁺.

### Tert-butyl N-[2-(4-formylcyclohexyl)-6-methoxy-indazol-5-yl]carbamate (Intermediate BGT)

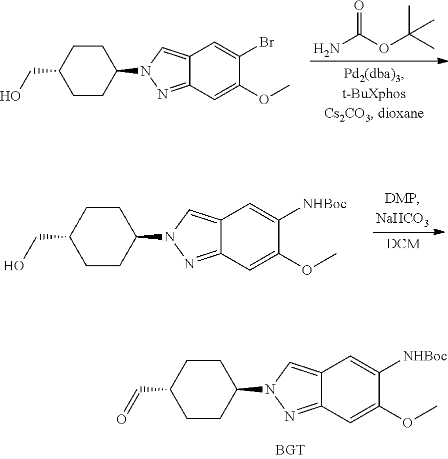

### Step 1—Tert-butyl N-[2-[4-(hydroxymethyl)cyclohexyl]-6-methoxy-indazol-5-yl]carbamate

A mixture of [4-(5-bromo-6-methoxy-indazol-2-yl)cyclohexyl]methanol (8.00 g, 23.6 mmol, synthesized via Steps 1-3 of Intermediate ATE), tert-butyl carbamate (4.14 g, 35.4 mmol), Pd₂(dba)₃ (2.16 g, 2.36 mmol), t-Bu Xphos (2.00 g, 4.72 mmol) and Cs₂CO₃ (15.4 g, 47.2 mmol) in dioxane (150 mL) was de-gassed and heated at 90° C. for 12 hrs. On completion, the mixture was concentrated in vacuo. The residue was purified by silica gel chromatography (SiO₂) and then re-purified by reverse phase (0.1% FA condition) to give the title compound (5.00 mg, 57% yield) as a white solid. LC-MS (ESI⁺) m/z 376.1 (M+H)⁺.

### Step 2—Tert-butyl N-[2-(4-formylcyclohexyl)-6-methoxy-indazol-5-yl]carbamate

To a mixture of tert-butyl N-[2-[4-(hydroxymethyl)cyclohexyl]-6-methoxy-indazol-5-yl]carbamate (800 mg, 2.13 mmol) and NaHCO₃ (716 mg, 8.52 mmol) in DCM (20 mL) was added DMP (1.36 g, 3.20 mmol). The reaction mixture was stirred at 20° C. for 2 hrs. On completion, the mixture was quenched with sat. Na₂S₂O₃ (50 mL) and NaHCO₃ (50 mL), stirred for 10 min, then extracted with DCM (2×50 mL). The organic layer was washed with brine (80 mL), dried in Na₂SO₄, filtered and the filtrate was concentrated in vacuo to give the title compound (700 mg, 88% yield) as a yellow solid. LC-MS (ESI⁺) m/z 374.2 (M+H)⁺.

### 3-[4-[4-[[4-(5-Amino-6-methoxy-indazol-2-yl)cyclohexyl]methyl-methyl-amino]-1-piperidyl]-3-methyl-2-oxo-benzimidazol-1-yl]piperidine-2,6-dione (Intermediate BGU)

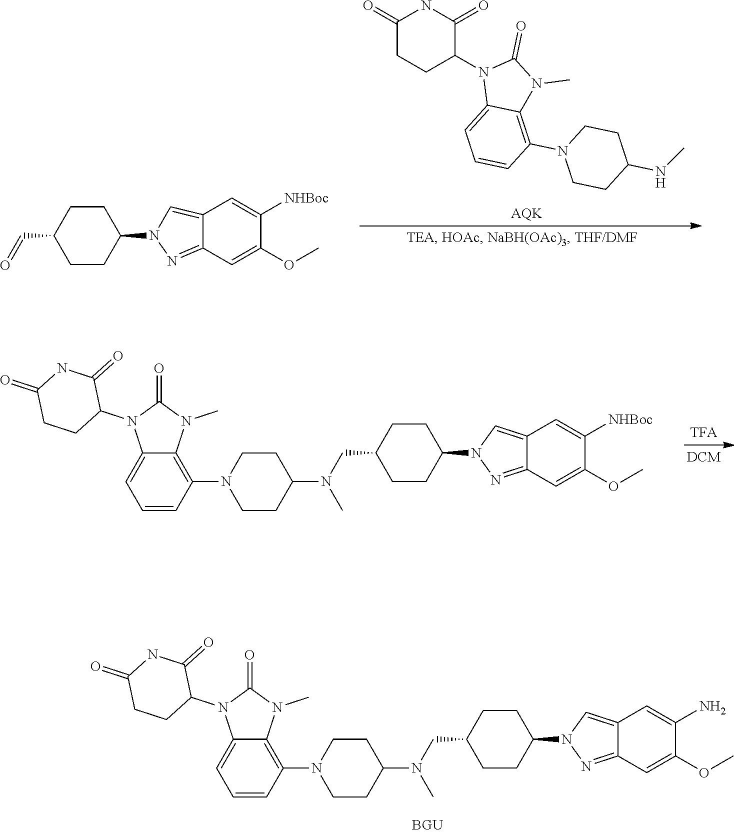

### Step 1—Tert-butyl N-[2-[4-[[[1-[1-(2,6-dioxo-3-piperidyl)-3-methyl-2-oxo-benzimidazol-4-yl]-4-piperidyl]-methyl-amino]methyl]cyclohexyl]-6-methoxy-indazol-5-yl]carbamate

To a solution of 3-[3-methyl-4-[4-(methylamino)-1-piperidyl]-2-oxo-benzimidazol-1-yl]piperidine-2,6-dione (873 mg, 2.14 mmol, HCl salt, Intermediate AQK) in a mixed solvent of DMF (10 mL) and THF (50 mL) was added KOAc (315 mg, 3.21 mmol). Then the mixture was cooled to 0° C. and tert-butyl N-[2-(4-formylcyclohexyl)-6-methoxy-indazol-5-yl]carbamate (800 mg, 2.14 mmol, Intermediate BGT) was added. Thirty minutes later, NaBH(OAc)₃ (907 mg, 4.28 mmol) was added into the above mixture and the reaction mixture was stirred at 0° C. for 3 hrs. On completion, the mixture was quenched with water (5 mL), concentrated in vacuo. The residue was purified by reverse phase (0.1% FA condition) to give the title compound (1.00 g, 64% yield) as a yellow solid. ¹H NMR (400 MHz, DMSO-d₆) δ 11.08 (s, 1H), 8.17 (s, 1H), 7.89 (s, 1H), 7.75 (s, 1H), 7.08-6.77 (m, 4H), 5.34 (dd, J=5.2, 12.8 Hz, 1H), 4.40-4.24 (m, 1H), 3.84 (s, 3H), 3.63 (s, 3H), 3.16-3.12 (m, 2H), 2.96-2.81 (m, 1H), 2.76-2.57 (m, 4H), 2.47-2.39 (m, 1H), 2.28-2.26 (m, 2H), 2.24 (s, 3H), 2.15-2.06 (s, 2H), 1.99-1.74 (m, 7H), 1.72-1.51 (m, 3H), 1.45 (s, 9H), 1.14-1.01 (m, 2H).

### Step 2—3-[4-[4-[[4-(5-Amino-6-methoxy-indazol-2-yl)cyclohexyl]methyl-methyl-amino]-1-piperidyl]-3-methyl-2-oxo-benzimidazol-1-yl]piperidine-2,6-dione

To a solution of tert-butyl N-[2-[4-[[[1-[1-(2,6-dioxo-3-piperidyl)-3-methyl-2-oxo-benzimidazol-4-yl]-4-piperidyl]-methyl-amino]methyl]cyclohexyl]-6-methoxy-indazol-5-yl]carbamate (60.0 mg, 82.3 umol) in TFA (2 mL) was added DCM (2 mL). The reaction mixture was stirred at 20° C. for 0.5 hr. On completion, the mixture was concentrated in vacuo to give the title compound (60.0 mg, 98% yield, TFA salt) as a yellow solid. LC-MS (ESI⁺) m/z 629.4 (M+H)⁺.

### 6-(Trifluoromethyl)pyridazine-4-carboxylic acid (Intermediate BQJ)

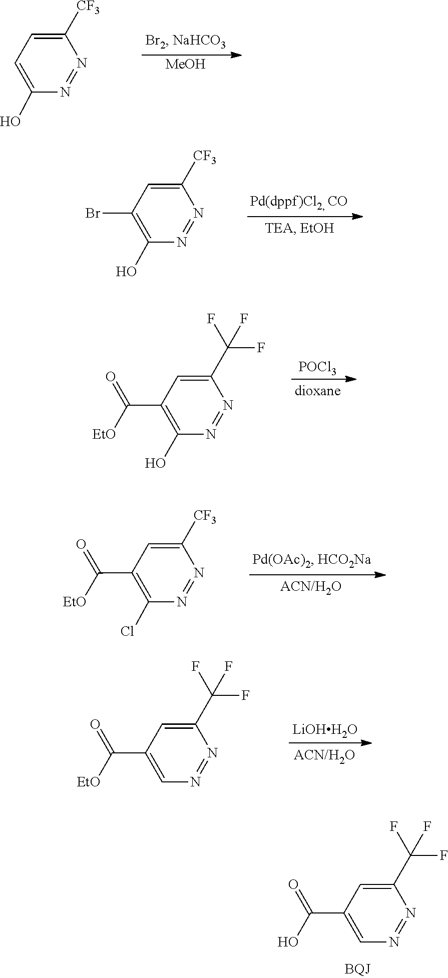

### Step 1—4-Bromo-6-(trifluoromethyl)pyridazin-3-ol

To a solution of 6-(trifluoromethyl)pyridazin-3-ol (3.00 g, 18.3 mmol) (CAS #174607-36-4), NaHCO₃ (4.61 g, 54.8 mmol, 2.13 mL) in MeOH (50 mL) was added Br₂ (4.38 g, 27.4 mmol) slowly. The reaction mixture was then stirred at 20° C. for 12 hrs. On completion, The reaction mixture was filtered and concentrated in vacuo. The crude was diluted with ethyl acetate (80 mL), washed with brine (20 mL), and dried over anhydrous sodium sulfate. The mixture was filtered and concentrated in vacuo to give the title compound (4.00 g, 90% yield) as a yellow solid. ¹H NMR (400 MHz, CDCl₃) δ 11.61-11.26 (m, 1H), 7.92 (s, 1H) LC-MS (ESI⁺) m/z 243.0 (M+H)⁺.

### Step 2—Ethyl 3-hydroxy-6-(trifluoromethyl)pyridazine-4-carboxylate

To a solution of 4-bromo-6-(trifluoromethyl)pyridazin-3-ol (4 g, 16.5 mmol) in EtOH (50 mL) was added Pd(dppf)Cl₂·CH₂Cl₂ (1.34 g, 1.65 mmol) and TEA (8.33 g, 82.3 mmol, 11.5 mL) under N₂ atmosphere. The suspension was degassed and purged with CO three times. The mixture was stirred under CO (50 psi) at 80° C. for 16 hrs. On completion, the reaction mixture was filtered and concentrated in vacuo. The residue was purified by silica gel chromatography (PE:EA=10:1 to 4:1) to give the title compound (3.10 g, 80% yield) as a white solid. ¹H NMR (400 MHz, CDCl₃) δ 12.15-11.26 (m, 1H), 8.07 (s, 1H), 4.47 (q, J=7.2 Hz, 2H), 1.44 (t, J=7.2 Hz, 3H).

### Step 3—Ethyl 3-chloro-6-(trifluoromethyl)pyridazine-4-carboxylate

To a solution of ethyl 3-hydroxy-6-(trifluoromethyl)pyridazine-4-carboxylate (1.4 g, 5.93 mmol) in dioxane (5 mL) was added POCl₃ (7.22 g, 47.0 mmol, 4.38 mL). The reaction mixture was then heated to 100° C. and stirred for 4 hrs. On completion, the reaction was quenched with water (30 mL) and basified with sodium bicarbonate solution (60 mL) and extracted with ethyl acetate (2×50 mL). The combined organic layers were washed with brine (30 mL) and dried over anhydrous sodium sulfate, filtered and the filtrate was concentrated in vacuo. The residue was purified by silica gel chromatography (PE:EA=10:1) to give the title compound (1.00 g, 66% yield) as a yellow liquid. ¹H NMR (400 MHz, CDCl₃) δ 8.15 (s, 1H), 4.53 (q, J=7.2 Hz, 2H), 1.47 (t, J=7.2 Hz, 3H), LC-MS (ESI⁺) m/z 255.0 (M+H)⁺.

### Step 4—Ethyl 6-(trifluoromethyl)pyridazine-4-carboxylate

To a solution of ethyl 3-chloro-6-(trifluoromethyl)pyridazine-4-carboxylate (1 g, 3.93 mmol) in a mixed solvent of ACN (5 mL) and H₂O (1 mL) was added HCOONa (400 mg, 5.88 mmol) and Pd(OAc)₂ (88.1 mg, 392 umol) under N₂. The suspension was then stirred at 105° C. for 4 hrs under N₂ atmosphere. On completion, the reaction mixture was filtered and the mixture solvent was purified by reversed-phase HPLC (0.10% HCl, condition) to give the title compound (200 mg, 23% yield) as a colorless oil. LC-MS (ESI⁺) m/z 221.1 (M+H)⁺.

### Step 5—6-(Trifluoromethyl)pyridazine-4-carboxylic acid

To a solution of ethyl 6-(trifluoromethyl)pyridazine-4-carboxylate (190 mg, 863 umol) in a mixed solvent of ACN (20 mL) and H₂O (50 mL) was added LiOH·H₂O (250 mg, 5.96 mmol) and the reaction mixture was stirred at 25° C. for 1 hr. On completion, the reaction mixture was acidified with HCl (1N) 6.0 mL, and extracted with ethyl acetate (3×50 mL). The combined organic layers were dried over anhydrous sodium sulfate, filtered and concentrated in vacuo to give the title compound (165 mg, 99% yield) as a white solid. ¹H NMR (400 MHz, DMSO-d₆) δ 9.80 (d, J=2.0 Hz, 1H), 8.39 (d, J=2.0 Hz, 1H), LC-MS (ESI⁺) m/z 193.1 (M+H)⁺.

### 3-(4-bromo-6-chloro-3-methyl-2-oxo-2,3-dihydro-1H-benzo[d]imidazol-1-yl)piperidine-2,6-dione (Intermediate BQN)

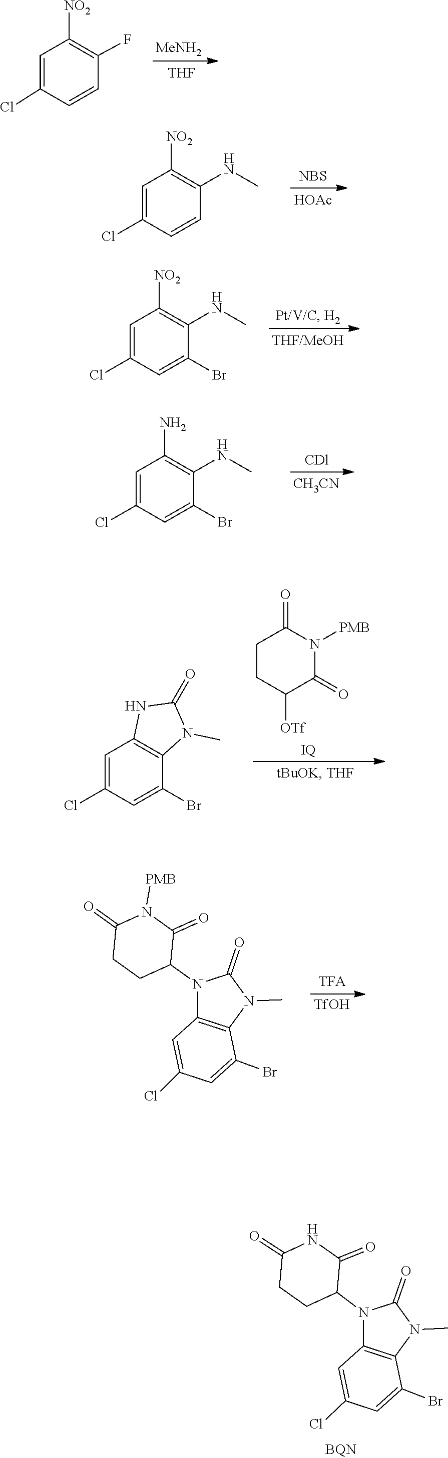

### Step 1—4-Chloro-N-methyl-2-nitroaniline

To a solution of MeNH₂ (2 M, 22.8 mL) in the THF (25 mL) was added 4-chloro-1-fluoro-2-nitro-benzene (2 g, 11.4 mmol, CAS #345-18-6) and DIEA (2.94 g, 22.8 mmol). The mixture was stirred at 60° C. for 4 hrs. On completion, the reaction mixture was concentrated in vacuo. The residue was diluted with EA (100 mL) and washed with water (3×100 mL). The organic layer was dried over Na₂SO₄, filtered and concentrated in vacuo to give the title compound (2.1 g, 99% yield) as yellow solid. ¹H NMR (400 MHz, CDCl₃) δ 8.18 (d, J=2.4 Hz, 1H), 8.02 (s, 1H), 7.42 (dd, J=2.4, 9.2 Hz, 1H), 6.82 (d, J=9.2 Hz, 1H), 3.04 (s, 3H).

### Step 2—2-Bromo-4-chloro-N-methyl-6-nitroaniline

A solution of 4-chloro-N-methyl-2-nitro-aniline (2 g, 10.7 mmol) and NBS (2.86 g, 16.0 mmol) in the AcOH (20 mL) was stirred at 20° C. for 16 hrs. On completion, the reaction mixture was diluted with EA (200 mL) and washed with brine (100 mL), and NaHCO₃ (2×100 mL). The organic layer was dried over Na₂SO₄, filtered and concentrated in vacuo. The residue was purified by column chromatography (petroleum ether/ethyl acetate=1/0 to 50/1) to give the title compound (2.5 g, 88% yield) as yellow solid. ¹H NMR (400 MHz, CDCl₃) δ 7.89 (d, J=2.4 Hz, 1H), 7.68 (d, J=2.4 Hz, 1H), 6.18 (s, 1H), 3.02 (s, 3H).

### Step 3—6-Bromo-4-chloro-N1-methylbenzene-1,2-diamine

To a solution of 2-bromo-4-chloro-N-methyl-6-nitro-aniline (2.5 g, 9.42 mmol) in the THF (10 mL) and MeOH (10 mL) was added Pt/V/C (300 mg, 10 wt %). The mixture was stirred at 20° C. for 12 hrs under H₂ (15 psi). On completion, the reaction mixture was filtered and concentrated in vacuo and purified by column chromatography (PE:EA=1:0 to 50:1) to give the title compound (1.68 g, 76% yield) as yellow oil. ¹H NMR (400 MHz, DMSO-d₆) δ 6.90 (d, J=2.4 Hz, 1H), 6.64 (d, J=2.4 Hz, 1H), 4.10 (s, 2H), 3.17 (s, 1H), 2.64 (s, 3H).

### Step 4—7-Bromo-5-chloro-1-methyl-1H-benzo[d]imidazol-2(3H)-one

To a solution of 3-bromo-5-chloro-N2-methyl-benzene-1,2-diamine (1.56 g, 6.62 mmol) in the MeCN (20 mL) was added CDI (1.61 g, 9.94 mmol). The mixture was stirred at 90° C. for 16 hrs. On completion, the reaction mixture was concentrated in vacuo. The residue was diluted with water (50 mL) and filtered to give the cake as title compound (1.42 g, 82% yield) as off-white solid. ¹H NMR (400 MHz, CDCl₃) δ 11.36 (s, 1H), 7.24 (d, J=2.0 Hz, 1H), 7.01 (d, J=2.0 Hz, 1H), 3.54 (s, 3H).

### Step 5—3-(4-Bromo-6-chloro-3-methyl-2-oxo-2,3-dihydro-1H-benzo[d]imidazol-1-yl)-1-(4-methoxybenzyl)piperidine-2,6-dione

To a solution of 4-bromo-6-chloro-3-methyl-1H-benzimidazol-2-one (1.42 g, 5.43 mmol) in the THF (20 mL) was added tBuOK (914 mg, 8.15 mmol) at −10-0° C. The mixture was stirred for 20 mins. Then a solution of [1-[(4-methoxyphenyl)methyl]-2,6-dioxo-3-piperidyl]trifluoromethanesulfonate (3.11 g, 8.15 mmol, Intermediate IQ) in the THF (5 mL) was added for 40 mins at −10-0° C. On completion, the reaction mixture was acidified with FA to pH=5˜6. Then the mixture was diluted with EA (150 mL) and washed with water (100 mL) and brine (100 mL). The organic layer was dried over Na₂SO₄, filtered and concentrated in vacuo and purified by column chromatography (petroleum ether/ethyl acetate=10/1 to 3/1) to give the title compound (2.0 g, 75% yield) as white solid. ¹H NMR (400 MHz, CDCl₃) δ 7.36 (d, J=8.4 Hz, 2H), 7.23 (d, J=1.6 Hz, 1H), 6.84 (d, J=8.4 Hz, 2H), 6.46 (s, 1H), 5.19 (dd, J=5.2, 13.6 Hz, 1H), 5.03-4.91 (m, 2H), 3.79 (s, 3H), 3.76 (s, 3H), 3.07-2.98 (m, 1H), 2.89-2.78 (m, 1H), 2.59 (dq, J=4.2, 13.2 Hz, 1H), 2.21-2.12 (m, 1H).

### Step 6—3-(4-bromo-6-chloro-3-methyl-2-oxo-2,3-dihydro-1H-benzo[d]imidazol-1-yl)piperidine-2,6-dione

To a solution of 3-(4-bromo-6-chloro-3-methyl-2-oxo-benzimidazol-1-yl)-1-[(4-methoxyphenyl) methyl]piperidine-2,6-dione (200 mg, 406 umol) in the TFA (2 mL) was added TfOH (0.4 mL). The mixture was stirred at 70° C. for 12 hrs. On completion, the reaction mixture was concentrated by N₂ and purified by reversed phase (0.1% HCl) to give the title compound (24.8 mg, 16% yield) as gray solid. ¹H NMR (400 MHz, DMSO-d₆) δ 11.14 (s, 1H), 7.42 (d, J=1.6 Hz, 1H), 7.36 (d, J=1.6 Hz, 1H), 5.42 (dd, J=5.2, 12.4 Hz, 1H), 3.62 (s, 3H), 2.90-2.80 (m, 1H), 2.80-2.70 (m, 1H), 2.66-2.60 (m, 1H), 2.07-1.98 (m, 1H). LC-MS (ESI⁺) m/z 374.0 (M+3)⁺.

### 3-[6-chloro-3-methyl-2-oxo-4-(4-piperidyl)benzimidazol-1-yl]piperidine-2,6-dione (Intermediate BQK)

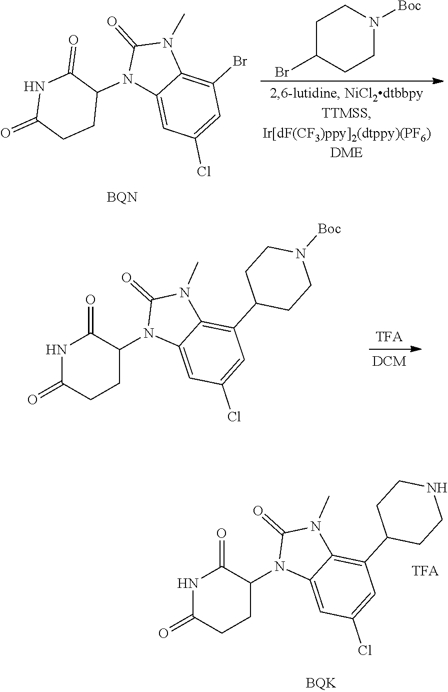 [SMILES: Cn1c(=O)n(C2CCC(=O)NC2=O)c2cc(Cl)cc(Br)c21 ; CC(C)(C)OC(=O)N1CCC(Br)CC1 ; Cn1c(=O)n(C2CCC(=O)NC2=O)c2cc(Cl)cc(C3CCN(C(=O)OC(C)(C)C)CC3)c21 ; Cn1c(=O)n(C2CCC(=O)NC2=O)c2cc(Cl)cc(C3CCNCC3)c21]

### Step 1—Tert-butyl 4-[6-chloro-1-(2,6-dioxo-3-piperidyl)-3-methyl-2-oxo-benzimidazol-4-yl]piperidine-1-carboxylate

To an 8 mL vial equipped with a stir bar was added 3-(4-bromo-6-chloro-3-methyl-2-oxo-benzimidazol-1-yl)piperidine-2,6-dione (600 mg, 1.61 mmol, Intermediate BQN), tert-butyl 4-bromopiperidine-1-carboxylate (425 mg, 1.61 mmol), Ir[dF(CF₃)ppy]₂(dtbpy)(PF₆) (18.0 mg, 16.10 umol), NiCl₂.dtbbpy (3.20 mg, 8.05 umol), TTMSS (400 mg, 1.61 mmol), and 2,6-lutidine (345 mg, 3.22 mmol) in DME (5 mL). The vial was sealed and placed under nitrogen. Then the reaction was stirred and irradiated with a 34 W blue LED lamp (7 cm away), with cooling fan to keep the reaction temperature at 25° C. for 14 hrs. On completion, the mixture was poured into the water (30 mL) and extracted with ethyl acetate (2×20 mL). The combined organic phase was washed with brine (2×30 mL), dried with anhydrous Na₂SO₄, filtered and concentrated in vacuo to give the residue. The residue was purified by reversed-phase HPLC (0.1% FA condition) to give the title compound (160 mg, 20% yield) as brown solid. LC-MS (ESI⁺) m/z 421.2 (M-55)*.

### Step 2—3-[6-Chloro-3-methyl-2-oxo-4-(4-piperidyl)benzimidazol-1-yl]piperidine-2,6-dione

To a solution of tert-butyl 4-[6-chloro-1-(2,6-dioxo-3-piperidyl)-3-methyl-2-oxo-benzimidazol-4-yl]piperidine-1-carboxylate (40.0 mg, 83.8 umol) in DCM (2 mL) was added TFA (19.1 mg, 167 umol). The reaction mixture was stirred at 25° C. for 0.5 hr. On completion, the mixture was concentrated in vacuo to give the title compound (40.0 mg, 97% yield) as brown oil. LC-MS (ESI⁺) m/z 377.3 (M+H)⁺.

### 3-[5-chloro-3-methyl-2-oxo-4-(4-piperidyl)benzimidazol-1-yl]piperidine-2,6-dione (Intermediate BQL)

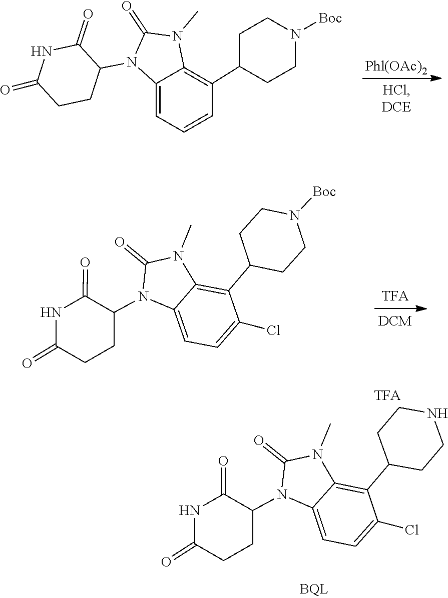 [SMILES: Cn1c(=O)n(C2CCC(=O)NC2=O)c2cccc(C3CCN(C(=O)OC(C)(C)C)CC3)c21 ; "Phl(OAc)2" ; Cn1c(=O)n(C2CCC(=O)NC2=O)c2ccc(Cl)c(C3CCN(C(=O)OC(C)(C)C)CC3)c21 ; Cn1c(=O)n(C2CCC(=O)NC2=O)c2ccc(Cl)c(C3CCNCC3)c21]

### Step 1—Tert-butyl 4-[5-chloro-1-(2,6-dioxo-3-piperidyl)-3-methyl-2-oxo-benzimidazol-4-yl] piperidine-1-carboxylate

To a solution of tert-butyl 4-[1-(2,6-dioxo-3-piperidyl)-3-methyl-2-oxo-benzimidazol-4-yl]piperidine-1-carboxylate (1.00 g, 2.26 mmol, synthesized via Steps 1-2 of Intermediate AZK) in DCE (50.0 mL) was added PhI(OAc)₂ (727 mg, 2.26 mmol) and HCl (1 M, 11.30 mL). The mixture was stirred at 50° C. for 12 hrs. On completion, the reaction mixture was washed with saturated solution of NaHCO₃ (2×50 mL) and saturated solution of Na₂S₂O₃ (2×50 mL). The organic layer was washed with brine (2×50 mL), then dried over anhydrous Na₂SO₄, filtered and concentrated in vacuo to give a residue. The residue was purified by reversed phase (0.1% FA condition) to give the crude product. The crude product was re-purified by prep-HPLC (column: Welch Ultimate XB-SiOH 250*50*10 um; mobile phase: [Hexane-EtOH]; B %: 1%-40%, 15 min) to give the title compound (250 mg, 23% yield) as a white solid. ¹H NMR (400 MHz, CDCl₃-d) δ 8.20 (s, 1H), 7.07 (d, J=8.4 Hz, 1H), 6.61 (d, J=8.4 Hz, 1H), 5.18 (dd, J=4.4, 12.0 Hz, 1H), 4.42-4.16 (m, 2H), 3.71 (s, 3H), 3.67-3.57 (m, 1H), 3.02-2.91 (m, 1H), 2.90-2.65 (m, 6H), 2.27-2.18 (m, 1H), 1.50 (s, 9H).

### Step 2—3-[5-Chloro-3-methyl-2-oxo-4-(4-piperidyl)benzimidazol-1-yl]piperidine-2,6-dione

To a solution of tert-butyl 4-[5-chloro-1-(2,6-dioxo-3-piperidyl)-3-methyl-2-oxo-benzimidazol-4-yl]piperidine-1-carboxylate (30.0 mg, 62.9 umol) in DCM (2.00 mL) was added TFA (770 mg, 6.75 mmol, 0.50 mL). The mixture was stirred at 25° C. for 0.5 hr. On completion, the reaction mixture was concentrated in vacuo to give the title compound (30.0 mg, 97% yield, TFA) as a white solid. LC-MS (ESI⁺) m/z 377.3 (M+H)⁺.

### 3-[3,5-Dimethyl-2-oxo-4-(4-piperidyl)benzimidazol-1-yl]piperidine-2,6-dione (Intermediate BQM)

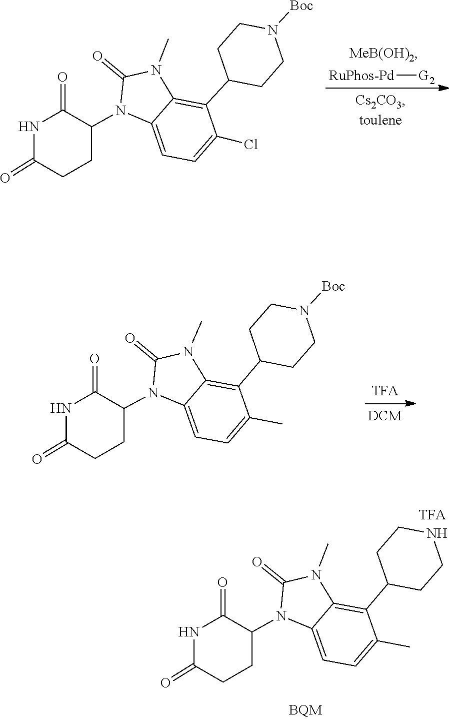

### Step 1—Tert-butyl 4-[1-(2,6-dioxo-3-piperidyl)-3,5-dimethyl-2-oxo-benzimidazol-4-yl]piperidine-1-carboxylate

To a mixture of tert-butyl 4-[5-chloro-1-(2,6-dioxo-3-piperidyl)-3-methyl-2-oxo-benzimidazol-4-yl]piperidine-1-carboxylate (95.0 mg, 199 umol, synthesized via Step 1 of Intermediate BQL) in toluene (4 mL) was added MeB(OH)₂ (238 mg, 3.98 mmol), Cs₂CO₃ (194 mg, 597 umol) and [2-(2-aminophenyl)phenyl]-chloro-palladium; dicyclohexyl-[2-(2,6-diisopropoxyphenyl)phenyl]phosphane (15.4 mg, 19.9 umol). Then the reaction mixture was stirred at 100° C. for 12 hours. On completion, the reaction mixture was filtered and concentrated in vacuo. The residue was purified by prep-HPLC (Column Phenomenex luna C18 150*25 mm*10 um, Condition: water (0.225% FA)-ACN) to give the title compound (20.0 mg, 21% yield) as white solid. LC-MS (ESI⁺) m/z 479.1 (M+Na)⁺.

### Step 2—3-[3,5-Dimethyl-2-oxo-4-(4-piperidyl)benzimidazol-1-yl]piperidine-2,6-dione

To a mixture of tert-butyl 4-[1-(2,6-dioxo-3-piperidyl)-3,5-dimethyl-2-oxo-benzimidazol-4-yl]piperidine-1-carboxylate (20.0 mg, 43.8 umol) in DCM (3 mL) was added TFA (770 mg, 6.75 mmol, 0.5 mL) then the reaction mixture was stirred at 25° C. for 0.5 hour. On completion, the reaction mixture was concentrated in vacuo to give title compound (20.0 mg, 97% yield, TFA) as red oil. LC-MS (ESI⁺) m/z 357.0 (M+H)⁺.

### [4-(5-bromo-6-morpholino-indazol-2-yl)cyclohexyl]methanol (Intermediate AWW)

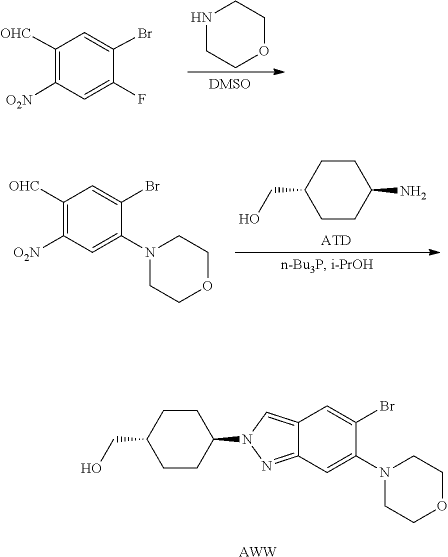

### Step 1—5-bromo-4-morpholino-2-nitro-benzaldehyde

A solution of 5-bromo-4-fluoro-2-nitro-benzaldehyde (2.00 g, 8.06 mmol, synthesized via Step 1 of Intermediate ATE) in DMSO (25 mL) was added with morpholine (2.81 g, 32.3 mmol). The reaction mixture was stirred at 80° C. for 1 hours. On completion, the reaction mixture was then diluted in ethyl acetate (50 mL) and water (50 mL), then extracted with ethyl acetate (3×50 mL). The organic layer was dried over Na₂SO₄, filtered and concentrated in vacuo to afford the residue. The residue was purified by column chromatography (PE:EA=4:1) to give the title compound (1.50 g, 58% yield) as yellow solid. ¹H NMR (400 MHz, CDCl₃) δ 10.33 (s, 1H), 8.21 (s, 1H), 7.63 (s, 1H), 4.07-3.82 (m, 4H), 3.32-3.24 (m, 4H). LC-MS (ESI⁺) m/z 314.9, 316.9 (M+H)⁺.

### Step 2—[4-(5-bromo-6-morpholino-indazol-2-yl)cyclohexyl]methanol

To a solution of 5-bromo-4-morpholino-2-nitro-benzaldehyde (1.50 g, 4.76 mmol) in IPA (50 mL) was added (4-aminocyclohexyl) methanol (738 mg, 5.71 mmol, Intermediate ATD). The mixture was heated at 80° C. for 4 hr under N₂. Then the reaction was cooled to 25° C., and tributylphosphine (2.89 g, 14.3 mmol) was added. The reaction mixture was heated to 80° C. for 16 hr. On completion, the reaction mixture was concentrated in vacuo to remove some IPA, and a solid was precipitated. The mixture was filtered and the filter cake was washed by petroleum ether to give the title compound (1.00 g, 53% yield) as yellow solid. ¹H NMR (400 MHz, CDCl₃) δ 7.93 (s, 1H), 7.86 (s, 1H), 7.32 (s, 1H), 4.39-4.32 (m, 1H), 4.02-3.86 (m, 4H), 3.58 (d, J=6.4 Hz, 2H), 3.10-3.09 (m, 4H), 2.51-2.22 (m, 2H), 2.15-1.89 (m, 4H), 1.78-1.64 (m, 2H), 1.34-1.25 (m, 2H).

### N-[2-(4-formylcyclohexyl)-6-morpholino-indazol-5-yl]-6-(trifluoromethyl)pyridine-2-carboxamide (Intermediate AYL)

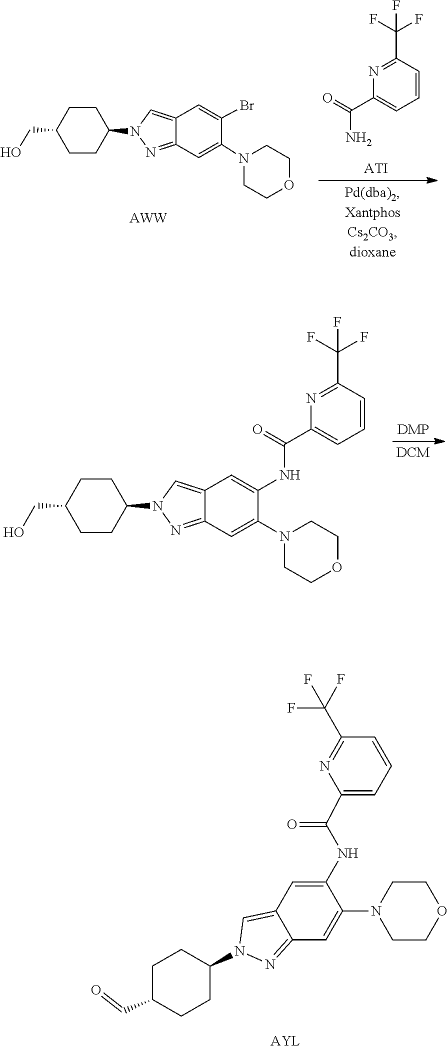

### Step 1—N-[2-[4-(hydroxymethyl)cyclohexyl]-6-morpholino-indazol-5-yl]-6-(trifluoromethyl) pyridine-2-carboxamide

To a solution of [4-(5-bromo-6-morpholino-indazol-2-yl)cyclohexyl]methanol (250 mg, 634 umol, Intermediate AWW) in dioxane (5.0 mL) was added Pd(dba)₂ (36.5 mg, 63.4 umol), Xantphos (73.4 mg, 127 umol), Cs₂CO₃ (413 mg, 1.27 mmol) and 6-(trifluoromethyl)pyridine-2-carboxamide (133 mg, 697 umol, Intermediate ATI). The mixture was stirred at 100° C. for 16 hours. On completion, the reaction mixture was filtered and concentrated in vacuo to give a residue. The residue was purified by reverse phase flash (0.1% FA condition) to give the title compound (100 mg, 31% yield) as a white solid. ¹H NMR (400 MHz, CDCl₃) δ 11.25 (s, 1H), 8.93 (s, 1H), 8.57 (d, J=8.0 Hz, 1H), 8.16 (t, J=8.0 Hz, 1H), 8.00-7.85 (m, 2H), 7.54 (s, 1H), 4.38 (tt, J=3.6, 12.0 Hz, 1H), 4.10-3.95 (m, 4H), 3.59 (d, J=6.4 Hz, 2H), 3.18-2.97 (m, 4H), 2.44-2.27 (m, 2H), 2.14-1.90 (m, 5H), 1.53-1.39 (m, 1H), 1.35-1.19 (m, 2H). LC-MS (ESI⁺) m/z 504.2 (M+H)⁺.

### Step 2—N-[2-(4-formylcyclohexyl)-6-morpholino-indazol-5-yl]-6-(trifluoromethyl)pyridine-2-carboxamide

To a mixture of N-[2-[4-(hydroxymethyl)cyclohexyl]-6-morpholino-indazol-5-yl]-6-(trifluoromethyl) pyridine-2-carboxamide (100 mg, 200 umol) in DCM (3.0 mL) was added DMP (126 mg, 298 umol) at 25° C. under N₂. The mixture was stirred at 25° C. for 1 hr. On completion, the reaction mixture was quenched with Na₂S₂O₃ (6.0 mL) and extracted with DCM (2×5.0 mL). The combined organic layer was washed with NaHCO₃ aqueous and brine (2×5.0 mL), dried with anhydrous Na₂SO₄, filtered and concentrated in vacuo to give the title compound (100 mg, 96% yield) as white solid. LC-MS (ESI⁺) m/z 502.2 (M+H)⁺.

### 3-[3,5-Dimethyl-4-[4-(methylamino)-1-piperidyl]-2-oxo-benzimidazol-1-yl]piperidine-2,6-dione (Intermediate BQV)

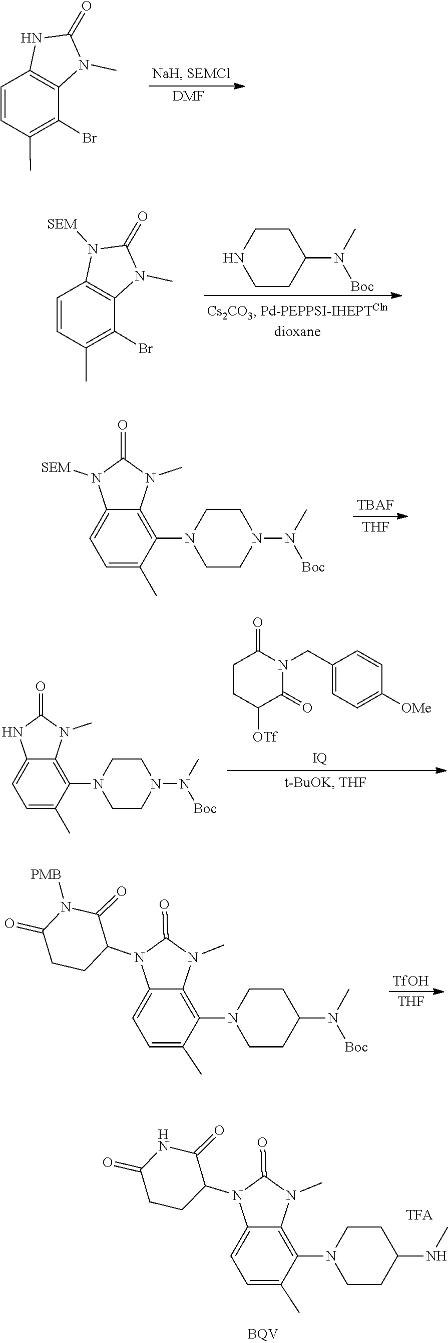

### Step 1—4-Bromo-3,5-dimethyl-1-((2-(trimethylsilyl)ethoxy)methyl)-1H-benzo[d]imidazol-2(3H)-one

To a solution of 4-bromo-3,5-dimethyl-1H-benzimidazol-2-one (1.00 g, 4.15 mmol, synthesized via Steps 1-5 of Intermediate BRG) in the DMF (10 mL) was added NaH (249 mg, 6.22 mmol, 60% dispersion in mineral oil) at 0° C. and the mixture was stirred for 30 mins. Then SEM-Cl (1.38 g, 8.30 mmol) was added and the mixture was stirred at 20° C. for 12 hrs. On completion, the reaction mixture was poured into ice water (50 mL) and extracted with EA (100 mL). The organic layer was dried over Na₂SO₄, filtered and concentrated in vacuo. The residue was purified by column chromatography (PE:EA=20:1) to give the title compound (900 mg, 58% yield) as white solid. ¹H NMR (400 MHz, CDCl₃) δ 7.04-6.96 (m, 2H), 5.30 (s, 2H), 3.80 (s, 3H), 3.63-3.56 (m, 2H), 2.44 (s, 3H), 0.95-0.89 (m, 2H), −0.02 (s, 9H).

### Step 2—Tert-butyl (1-(3,5-dimethyl-2-oxo-1-((2-(trimethylsilyl)ethoxy)methyl)-2,3-dihydro-1H-benzo[d]imidazol-4-yl)piperidin-4-yl)(methyl)carbamate

To a solution of 4-bromo-3,5-dimethyl-1-(2-trimethylsilylethoxymethyl)benzimidazol-2-one (500 mg, 1.35 mmol) and tert-butyl N-methyl-N-(4-piperidyl)carbamate (432 mg, 2.02 mmol) in dioxane (5 mL) was added Cs₂CO₃ (1.32 g, 4.04 mmol) and catalyst Pd-PEPPSI-IHEPT^{Cln} (40 mg, CAS #1612891-29-8). The mixture was stirred at 100° C. for 12 hours. On completion, the reaction mixture was concentrated under reduced pressure to remove the solvent. The residue was diluted with H₂O (10 mL) and extracted with EA (3×30 mL). The combined organic layers were washed with brine (10 mL), dried over Na₂SO₄, filtered and concentrated under reduced pressure to give a residue. The crude product was purified by reverse phase (0.1% FA condition) to give the title compound (120 mg, 16% yield) as a brown solid. ¹H NMR (400 MHz, CDCl₃) δ 6.91 (d, J=7.8 Hz, 1H), 6.80 (d, J=7.8 Hz, 1H), 5.28 (s, 2H), 3.77 (s, 3H), 3.64-3.57 (m, 2H), 3.46 (d, J=1.6 Hz, 2H), 3.09 (d, J=12.0 Hz, 2H), 2.81 (s, 3H), 2.41 (s, 3H), 1.87 (s, 2H), 1.68 (d, J=11.6 Hz, 2H), 1.50 (s, 9H), 0.96-0.89 (m, 2H), 0.88-0.73 (m, 1H), −0.03 (s, 9H). LC-MS (ESI⁺) m/z 505.5 (M+H)⁺.

### Step 3—Tert-butyl (1-(3,5-dimethyl-2-oxo-2,3-dihydro-1H-benzo[d]imidazol-4-yl)piperidin-4-yl) (methyl)carbamate

To a solution of tert-butyl N-[1-[3,5-dimethyl-2-oxo-1-(2-trimethylsilylethoxymethyl)benzimidazol-4-yl]-4-piperidyl]-N-methyl-carbamate (120 mg, 237 umol) in THF (2 mL) was added TBAF (621 mg, 2.38 mmol). The mixture was stirred at 80° C. for 12 hours. On completion, the reaction mixture was concentrated under reduced pressure to remove solvent and give a residue. The crude product was triturated with mixture of MeCN (0.5 mL) and H₂O (1 mL) at 20° C. for 60 minutes to give the title compound (60.0 mg, 65% yield) as an off-white solid. ¹H NMR (400 MHz, CDCl₃) δ 8.58 (s, 1H), 6.83-6.72 (m, 2H), 4.30-3.93 (m, 1H), 3.75 (s, 3H), 3.50-3.41 (m, 2H), 3.09 (m, 2H), 2.82 (s, 3H), 2.39 (s, 3H), 1.95-1.82 (m, 2H), 1.68 (m, 2H), 1.50 (s, 9H). LC-MS (ESI⁺) m/z 375.0 (M+H)⁺.

### Step 4—Tert-butyl N-[1-[1-[1-[(4-methoxyphenyl)methyl]-2,6-dioxo-3-piperidyl]-3,5-dimethyl-2-oxo-benzimidazol-4-yl]-4-piperidyl]-N-methyl-carbamate

To a solution of tert-butyl N-[1-(3,5-dimethyl-2-oxo-1H-benzimidazol-4-yl)-4-piperidyl]-N-methyl-carbamate (60.0 mg, 160 umol) in THF (2.00 mL) was added t-BuOK (26.9 mg, 240 umol) at −10° C. and the mixture was stirred at −10° C. for 1 hr. Then a solution of [1-[(4-methoxyphenyl)methyl]-2,6-dioxo-3-piperidyl]trifluoromethanesulfonate (91.6 mg, 240 umol, Intermediate IQ) in THF (1.00 mL) was added to the above solution and the mixture was stirred at −10° C. for 1 hr. On completion, the mixture was quenched with sat. NH₄Cl (15 mL) and extracted with EA (3×10 mL). The organic layers were washed with brine (2×10 mL), dried over anhydrous Na₂SO₄, filtered and concentrated in vacuo. The mixture was purified by reverse phase: (0.1% FA) to give the title compound (60.0 mg, 61% yield) as yellow solid. 1H NMR (400 MHz, DMSO-d₆) δ 7.20 (d, J=8.8 Hz, 2H), 6.91-6.78 (m, 2H), 6.75-6.65 (m, 2H), 5.52-5.38 (m, 1H), 4.89-4.69 (m, 2H), 4.13-3.79 (m, 1H), 3.72 (s, 3H), 3.65 (s, 3H), 3.05-2.99 (m, 3H), 2.83-2.77 (m, 1H), 2.74 (s, 3H), 2.72-2.68 (m, 1H), 2.53-2.51 (m, 2H), 2.39-2.33 (m, 3H), 2.05-1.97 (m, 1H), 1.95-1.83 (m, 2H), 1.63-1.48 (m, 2H), 1.42 (s, 9H). LC-MS (ESI⁺) m/z 606.5 (M+H)⁺.

### Step 5—3-[3,5-Dimethyl-4-[4-(methylamino)-1-piperidyl]-2-oxo-benzimidazol-1-yl]piperidine-2,6-dione

A solution of tert-butyl N-[1-[1-[1-[(4-methoxyphenyl)methyl]-2,6-dioxo-3-piperidyl]-3,5-dimethyl-2-oxo-benzimidazol-4-yl]-4-piperidyl]-N-methyl-carbamate (60.0 mg, 99.0 umol) in a mix solvent of TfOH (170 mg, 1.13 mmol) and TFA (1.23 g, 10.8 mmol). The mixture was stirred at 70° C. for 16 hrs. On completion, the mixture was concentrated in vacuo. The mixture was purified by prep-HPLC (GX-U, Phenomenex Luna C18 150*25 mm*10 um, water (0.1% TFA)-ACN, 5%-35%, 10 min) to give the title compound (35 mg, 91% yield) as white solid. ¹H NMR (400 MHz, DMSO-d₆) δ 11.07 (s, 1H), 8.61 (s, 1H), 6.92-6.83 (m, 1H), 6.81-6.73 (m, 1H), 5.37-5.25 (m, 1H), 3.62 (s, 3H), 3.30-3.25 (m, 2H), 3.15-3.03 (m, 3H), 2.94-2.81 (m, 1H), 2.73-2.62 (m, 2H), 2.62-2.58 (m, 3H), 2.33 (s, 3H), 2.05-1.91 (m, 3H), 1.83-1.65 (m, 2H). LC-MS (ESI⁺) m/z 386.1 (M+H)⁺.

### N-[2-(4-formylcyclohexyl)-6-methoxy-1-methyl-indol-5-yl]-6-(trifluoromethyl)pyridine-2-carboxamide (Intermediate BQW)

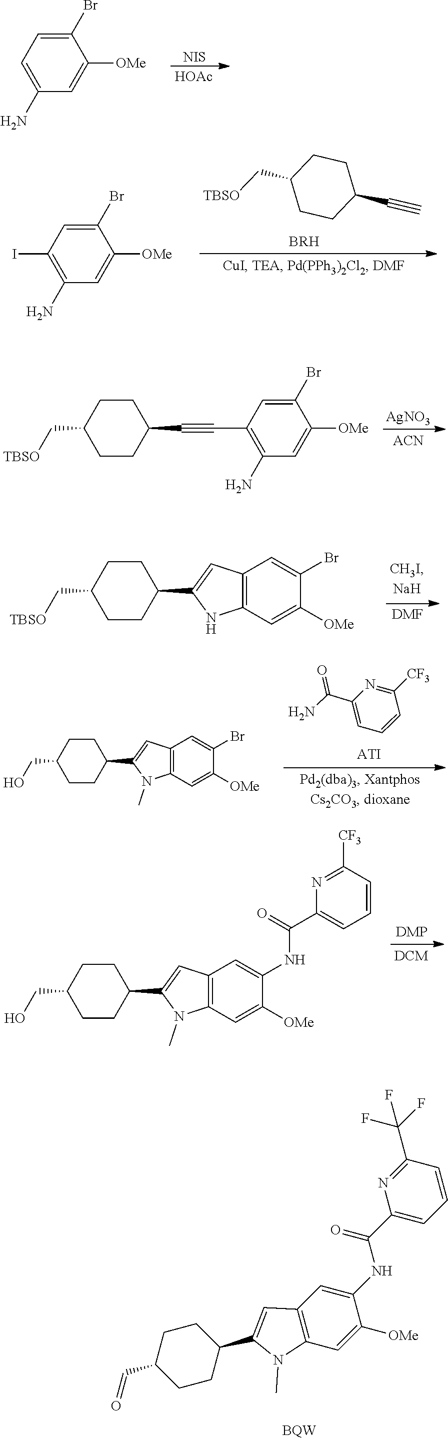

### Step 1—4-Bromo-2-iodo-5-methoxy-aniline

To a solution of 4-bromo-3-methoxy-aniline (3 g, 14.8 mmol, CAS #19056-40-7) in HOAc (40 mL) was added NIS (3.34 g, 14.8 mmol) and the mixture was stirred at 25° C. for 1.5 hrs. On completion, the mixture was poured into H₂O (300 mL) and stirred for 5 min. The mixture was then filtered and the solid was dried in vacuo to give the title compound (4.4 g, 90% yield) as gray solid. ¹H NMR (400 MHz, CDCl₃) δ 7.70 (s, 1H), 6.33 (s, 1H), 4.13 (s, 2H), 3.83 (s, 3H), LC-MS (ESI⁺) m/z 328.1 (M+H)⁺.

### Step 2—4-Bromo-2-[2-[4-[[tert-butyl(dimethyl)silyl]oxymethyl]cyclohexyl]ethynyl]-5-methoxy-aniline

To a solution of 4-bromo-2-iodo-5-methoxy-aniline (800 mg, 2.44 mmol), tert-butyl-[(4-ethynylcyclohexyl)methoxy]-dimethyl-silane (800 mg, 3.17 mmol, Intermediate BRH) in DMF (8.00 mL) was added CuI (9.29 mg, 48.7 umol), Pd(PPh3)₂Cl₂ (34.2 mg, 48.7 umol) and TEA (987 mg, 9.76 mmol). The mixture was stirred at 25° C. for 3 hrs. On completion, the mixture was diluted with H₂O (50 mL) and extracted with EA (3×30 mL). The organic layers were washed with brine (2×30 mL), dried over anhydrous Na₂SO₄, filtered and concentrated in vacuo. The mixture was purified by reverse phase: (0.1% FA) to give the title compound (668 mg, 60% yield) as white solid. ¹H NMR (400 MHz, CDCl₃) δ 7.38 (s, 1H), 6.24 (s, 1H), 3.84 (s, 3H), 3.42 (d, J=6.0 Hz, 2H), 2.48-2.36 (m, 1H), 2.12-2.05 (m, 2H), 1.86-1.80 (m, 2H), 1.47-1.42 (m, 2H), 1.05-0.91 (m, 3H), 0.90 (s, 9H), 0.04 (s, 6H), LC-MS (ESI⁺) m/z 453.9 (M+H)⁺.

### Step 3—[4-(5-Bromo-6-methoxy-1H-indol-2-yl)cyclohexyl] methoxy-tert-butyl-dimethyl-silane

To a solution of 4-bromo-2-[2-[4-[[tert-butyl(dimethyl)silyl]oxymethyl]cyclohexyl]ethynyl]-5-methoxy-aniline (668 mg, 1.48 mmol) in ACN (5 mL) was added AgNO₃ (25.0 mg, 147 umol) and the mixture was stirred at 80° C. for 16 hrs. On completion, the mixture was filtered and concentrated in vacuo. The mixture was purified by silica gel column (PE:EA=5:1) to give the title compound (540 mg, 80% yield) as yellow solid. ¹H NMR (400 MHz, CDCl₃) δ 7.85 (s, 1H), 7.67 (s, 1H), 6.87 (s, 1H), 6.10 (s, 1H), 3.90 (s, 3H), 3.47 (d, J=6.0 Hz, 2H), 2.69-2.57 (m, 1H), 2.17-2.06 (m, 2H), 1.98-1.87 (m, 2H), 1.54-1.45 (m, 3H), 1.19-1.05 (m, 2H), 0.91 (s, 9H), 0.06 (s, 6H), LC-MS (ESI⁺) m/z 454.0 (M+H)⁺.

### Step 4—[4-(5-Bromo-6-methoxy-1-methyl-indol-2-yl)cyclohexyl]methanol

To a solution of [4-(5-bromo-6-methoxy-1H-indol-2-yl)cyclohexyl]methoxy-tert-butyl-dimethyl-silane (490 mg, 1.08 mmol) in DMF (5.00 mL) was added NaH (51.9 mg, 1.30 mmol, 60% dispersion in mineral oil) at 0° C. The mixture was stirred at 0° C. for 0.5 hr, then CH₃I (307 mg, 2.17 mmol) was added and the mixture was stirred at 25° C. for 16 hrs. On completion, the mixture was diluted with H₂O (40 mL), and extracted with EA (3×20 mL). The organic layers were washed with brine (2×20 mL), dried over anhydrous Na₂SO₄, filtered and concentrated in vacuo. The mixture was purified by reverse phase: (0.1% FA) to give the title compound (300 mg, 78% yield) as yellow solid. ¹H NMR (400 MHz, DMSO-d₆) δ 7.59 (s, 1H), 7.11 (s, 1H), 6.07 (s, 1H), 4.42 (t, J=5.2 Hz, 1H), 3.85 (s, 3H), 3.65 (s, 3H), 3.28-3.23 (m, 2H), 2.72-2.60 (m, 1H), 2.04-1.92 (m, 2H), 1.90-1.76 (m, 2H), 1.48-1.28 (m, 3H), 1.17-1.02 (m, 2H), LC-MS (ESI⁺) m/z 353.9 (M+H)⁺.

### Step 5—N-[2-[4-(hydroxymethyl)cyclohexyl]-6-methoxy-1-methyl-indol-5-yl]-6-(trifluoromethyl) pyridine-2-carboxamide

To a solution of [4-(5-bromo-6-methoxy-1-methyl-indol-2-yl)cyclohexyl]methanol (280 mg, 794 umol), 6-(trifluoromethyl)pyridine-2-carboxamide (181 mg, 953 umol, Intermediate ATI) in dioxane (5 mL) was added Pd₂(dba)₃ (72.7 mg, 79.4 umol), Xantphos (91.9 mg, 158 umol) and Cs₂CO₃ (776 mg, 2.38 mmol) under N₂. The mixture was then stirred at 80° C. for 40 hr under N₂. On completion, the mixture was filtered and concentrated in vacuo. The mixture was purified by reverse phase: (0.1% FA) to give the title compound (150 mg, 40% yield) as yellow solid. ¹H NMR (400 MHz, DMSO-d₆) δ 10.36 (s, 1H), 8.49 (s, 1H), 8.47-8.42 (m, 1H), 8.42-8.36 (m, 1H), 8.19 (dd, J=1.2, 7.6 Hz, 1H), 7.17 (s, 1H), 6.16 (s, 1H), 4.43 (t, J=5.2 Hz, 1H), 3.98 (s, 3H), 3.69 (s, 3H), 3.30-3.25 (m, 2H), 2.74-2.65 (m, 1H), 2.05-1.96 (m, 2H), 1.92-1.80 (m, 2H), 1.49-1.34 (m, 3H), 1.18-1.05 (m, 2H), LC-MS (ESI⁺) m/z 462.1 (M+H)⁺.

### Step 6—N-[2-(4-formylcyclohexyl)-6-methoxy-1-methyl-indol-5-yl]-6-(trifluoromethyl)pyridine-2-carboxamide

To a solution of N-[2-[4-(hydroxymethyl)cyclohexyl]-6-methoxy-1-methyl-indol-5-yl]-6-(trifluoromethyl)pyridine-2-carboxamide (50.0 mg, 108 umol) in DCM (3.00 mL) was added DMP (68.9 mg, 162 umol) and the mixture was stirred at 25° C. for 1 hr. On completion, the mixture was diluted with DCM (20 mL), quenched with saturated Na₂S₂O₃ (15 mL), and washed with saturated NaHCO₃ (2×20 mL). The organic layer was dried over anhydrous Na₂SO₄, filtered and concentrated in vacuo. The mixture was purified by prep-HPLC (Phenomenex luna C18 150*25 mm*10 um, water (0.225% FA)-ACN, B %: 60%-90%, 11 min) to give the title compound (25 mg, 50% yield) as yellow solid. ¹H NMR (400 MHz, DMSO-d₆) δ 10.36 (s, 1H), 9.64 (s, 1H), 8.50 (s, 1H), 8.47-8.43 (m, 1H), 8.42-8.36 (m, 1H), 8.19 (d, J=7.6 Hz, 1H), 7.18 (s, 1H), 6.18 (s, 1H), 3.98 (s, 3H), 3.70 (s, 3H), 2.77-2.72 (m, 1H), 2.40-2.35 (m, 1H), 2.11-2.01 (m, 4H), 1.54-1.39 (m, 4H), LC-MS (ESI⁺) m/z 460.1 (M+H)⁺.

### 3-(5-methoxy-3-methyl-4-(4-(methylamino)piperidin-1-yl)-2-oxo-2,3-dihydro-1H-benzo[d]imidazol-1-yl)piperidine-2,6-dione (Intermediate BQX)

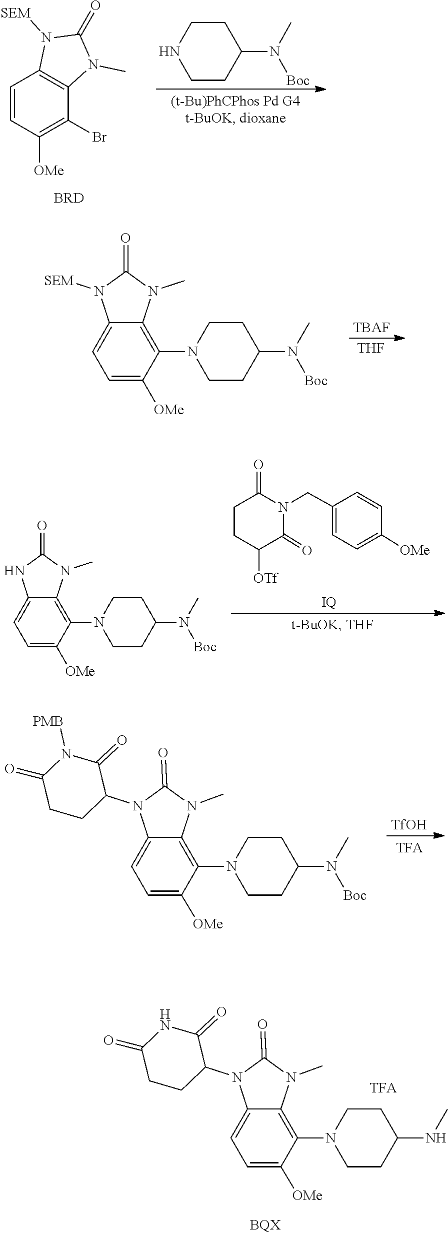

### Step 1—Tert-butyl N-[1-[5-methoxy-3-methyl-2-oxo-1-(2-trimethylsilylethoxymethyl)benzimidazol-4-yl]-4-piperidyl]-N-methyl-carbamate

A solution of 4-bromo-5-methoxy-3-methyl-1-(2-trimethylsilylethoxymethyl)benzimidazol-2-one (250 mg, 645 umol, Intermediate BRD) tert-butyl N-methyl-N-(4-piperidyl)carbamate (276 mg, 1.29 mmol, CAS #108612-54-0), (t-Bu)PhCPhos Pd G4 (35 mg, 64.5 umol) and t-BuOK (217 mg, 1.94 mmol) in dioxane (8 mL) was stirred at 85° C. under N₂ for 16 hrs. On completion, the reaction mixture was diluted with EtOAC (40 mL), and washed with brine (2×20 mL). The organic layer was separated and dried over Na₂SO₄ and concentrated in vacuo. The residue was purified by reverse phase (FA condition) to give the title compound (35.0 mg, 10% yield). ¹H NMR (400 MHz, MeOD) δ 7.04 (d, J=8.8 Hz, 1H), 6.79 (d, J=8.8 Hz, 1H), 5.31 (s, 2H), 3.89 (s, 3H), 3.81 (s, 3H), 3.67-3.53 (m, 4H), 3.39 (s, 1H), 3.06-2.99 (m, 2H), 2.87 (s, 3H), 2.09-1.92 (m, 2H), 1.68 (d, J=12 Hz, 2H), 1.53 (s, 9H), 1.39-1.31 (m, 2H), 0.98-0.89 (m, 2H), 0.00 (s, 9H).

### Step 2—Tert-butyl (1-(5-methoxy-3-methyl-2-oxo-2,3-dihydro-1H-benzo[d]imidazol-4-yl)piperidin-4-yl)(methyl)carbamate

To a solution of tert-butyl N-[1-[5-methoxy-3-methyl-2-oxo-1-(2-trimethylsilylethoxymethyl) benzimidazol-4-yl]-4-piperidyl]-N-methyl-carbamate (40.0 mg, 76.8 umol) in THF (5 mL) was added TBAF (200 mg, 768 umol) at 25° C. The reaction was warmed to 70° C. and stirred for 16 hrs. On completion, the reaction mixture was diluted with EtOAc (60 mL), and washed with brine (4×30 mL). The organic layer was separated, dried over Na₂SO₄ and concentrated in vacuo to give the title compound (29.0 mg, 96% yield) as brown solid. ¹H NMR (400 MHz, DMSO-d₆) δ 6.78 (d, J=8.4 Hz, 1H), 6.67 (d, J=8.4 Hz, 1H), 3.82 (s, 3H), 3.61 (s, 3H), 3.38-3.20 (m, 2H), 3.01-2.90 (m, 2H), 2.79 (s, 3H), 1.97-1.82 (m, 2H), 1.64-1.51 (m, 2H), 1.48 (s, 9H).

### Step 3—Tert-butyl (1-(5-methoxy-1-(1-(4-methoxybenzyl)-2,6-dioxopiperidin-3-yl)-3-methyl-2-oxo-2,3-dihydro-1H-benzo[d]imidazol-4-yl)piperidin-4-yl)(methyl)carbamate

To a solution of tert-butyl N-[1-(5-methoxy-3-methyl-2-oxo-1H-benzimidazol-4-yl)-4-piperidyl]-N-methyl-carbamate (24.0 mg, 61.4 umol) in THF (3 mL) was added tBuOK (13.8 mg, 122 umol) at 0° C. and the mixture was stirred at 0° C. for 30 min. Then, a solution of [1-[(4-methoxyphenyl) methyl]-2,6-dioxo-3-piperidyl]trifluoromethanesulfonate (37.5 mg, 98.3 umol, Intermediate IQ) in THF (0.5 mL) was added into the mixture slowly and the reaction was stirred at 0° C. for 2 hrs. On completion, the reaction mixture was quenched with saturated NH₄Cl solution (1 mL), and diluted with water (10 mL). The mixture was then extracted with EtOAc (2×40 mL). The organic layer was separated, dried over Na₂SO₄ and concentrated in vacuo. The residue was purified by prep-TLC (DCM:EtOAc=3:1, Rf=0.5) to give the title compound (36 mg, 94% yield) as yellow solid. LC-MS (ESI⁺) m/z 622.2 (M+H)⁺.

### Step 4—3-(5-methoxy-3-methyl-4-(4-(methylamino)piperidin-1-yl)-2-oxo-2,3-dihydro-1H-benzo[d]imidazol-1-yl)piperidine-2,6-dione

To a solution of tert-butyl N-[1-[5-methoxy-1-[1-[(4-methoxyphenyl)methyl]-2,6-dioxo-3-piperidyl]-3-methyl-2-oxo-benzimidazol-4-yl]-4-piperidyl]-N-methyl-carbamate (30.0 mg, 48.2 umol) in TFA (0.4 mL) was added TfOH (0.08 mL) at 25° C. The reaction mixture was warmed to 70° C. and stirred for 3 hrs. On completion, the reaction mixture was concentrated in vacuo. The residue was purified by prep-HPLC (column: Phenomenex luna C18 150*25 mm*10 um; mobile phase: [water (0.2250% FA)-ACN]; B %: 3%-33%, 11 min) to give the title compound (11.0 mg, 56% yield) as white solid. LC-MS (ESI⁺) m/z 402.1 (M+H)⁺.

### N-[2-(4-formylcyclohexyl)-6-[(1R,4R)-2-oxa-5-azabicyclo[2.2.1]heptan-5-yl]indazol-5-yl]-6-(trifluoromethyl)pyridine-2-carboxamide (Intermediate BQY)

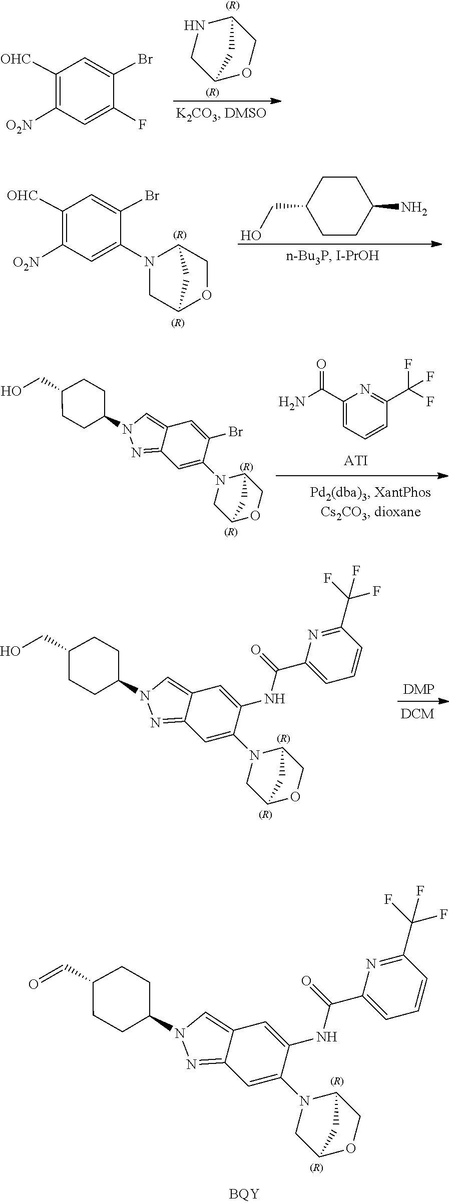

### Step 1—5-Bromo-2-nitro-4-[(1R,4R)-2-oxa-5-azabicyclo[2.2.1]heptan-5-yl]benzaldehyde

A solution of (1R,4R)-2-oxa-5-azabicyclo[2.2.1]heptane (1.20 g, 12.1 mmol, CAS #661470-56-0), K₂CO₃ (2.09 g, 15.1 mmol) and 5-bromo-4-fluoro-2-nitro-benzaldehyde (1.50 g, 6.05 mmol, CAS #213382-45-7) in DMSO (10 mL) was stirred at 80° C. for 2 hrs. On completion, the reaction was diluted with EtOAc (100 mL) and washed with brine (4×40 mL). The organic layer was separated, dried over Na₂SO₄, filtered and the filtrate was concentrated in vacuo to give the title compound (1.9 g, 90% yield) as yellow solid. ¹H NMR (400 MHz, CDCl₃) δ 10.14 (s, 1H), 8.09 (s, 1H), 7.14 (s, 1H), 4.75 (d, J=1.6 Hz, 1H), 4.64 (s, 1H), 4.03-3.99 (m, 2H), 3.91 (dd, J=1.6, 8.0 Hz, 1H), 3.40 (dd, J=0.8, 10.4 Hz, 1H), 2.01-1.98 (m, 2H).

### Step 2—[4-[5-Bromo-6-[(1R,4R)-2-oxa-5-azabicyclo[2.2.1]heptan-5-yl]indazol-2-yl]cyclohexyl]methanol

To a solution of 5-bromo-2-nitro-4-[(1R,4R)-2-oxa-5-azabicyclo[2.2.1]heptan-5-yl]benzaldehyde (1.70 g, 5.20 mmol) in i-PrOH (30 mL) was added (4-aminocyclohexyl)methanol (805 mg, 6.24 mmol CAS #1467-84-1). The mixture was heated to 80° C. and stirred for 15 hrs. Then, the mixture was cooled to 25° C. and tri-n-butylphosphane (3.15 g, 15.5 mmol) was added. The reaction mixture was heated to 80° C. and stirred for 16 hrs. On completion, the reaction was concentrated in vacuo. The residue was purified by prep-HPLC (column: Phenomenex luna C18 (250*70 mm, 15 um); mobile phase: [water (0.225% FA)-ACN]; B %: 35%-65%, 20 min) to give the title compound (4.00 g, 81% yield) as a yellow solid. ¹H NMR (400 MHz, CDCl₃) δ 7.88 (s, 1H), 7.79 (d, J=0.8 Hz, 1H), 7.15 (s, 1H), 4.61 (s, 1H), 4.36-4.26 (m, 2H), 4.22 (d, J=7.6 Hz, 1H), 3.83 (dd, J=2.0, 7.6 Hz, 1H), 3.63 (dd, J=1.8, 10.8 Hz, 1H), 3.55 (d, J=6.4 Hz, 2H), 3.45 (d, J=10.4 Hz, 1H), 2.33-2.27 (m, 2H), 2.08-1.98 (m, 4H), 1.98-1.89 (m, 3H), 1.75-1.59 (m, 1H), 1.30-1.17 (m, 2H).

### Step 3—N-[2-[4-(hydroxymethyl)cyclohexyl]-6-[(1R,4R)-2-oxa-5-azabicyclo[2.2.1]heptan-5-yl]indazol-5-yl]-6-(trifluoromethyl)pyridine-2-carboxamide

To a solution of [4-[5-bromo-6-[(1R,4R)-2-oxa-5-azabicyclo[2.2.1]heptan-5-yl]indazol-2-yl]cyclohexyl]methanol (150 mg, 369 umol) in dioxane (4.5 mL) was added Pd₂(dba)₃ (33.8 mg, 36.9 umol), Xantphos (42.7 mg, 73.8 umol), Cs₂CO₃ (240 mg, 738 umol) and 6-(trifluoromethyl) pyridine-2-carboxamide (105 mg, 553 umol, Intermediate ATI). The mixture was stirred at 110° C. for 16 hrs under N₂. On completion, the reaction mixture was filtered and concentrated in vacuo. The residue was purified by reverse phase flash (0.1% FA condition) to give the title compound (160 mg, 84% yield) as white solid. ¹H NMR (400 MHz, DMSO-d6) δ 10.69 (s, 1H), 8.67 (s, 1H), 8.50-8.46 (m, 1H), 8.41 (t, J=7.6 Hz, 1H), 8.34 (s, 1H), 8.21 (dd, J=0.8, 7.6 Hz, 1H), 7.39 (s, 1H), 4.61 (s, 1H), 4.49 (t, J=5.2 Hz, 1H), 4.42-4.33 (m, 1H), 4.07-3.97 (m, 2H), 3.66 (dd, J=1.6, 7.6 Hz, 1H), 3.42 (d, J=9.6 Hz, 1H), 3.28-3.22 (m, 2H), 2.20-2.05 (m, 3H), 1.95-1.81 (m, 5H), 1.51-1.48 (m, 1H), 1.21-1.08 (m, 2H).

### Step 4—N-[2-(4-formylcyclohexyl)-6-[(1R,4R)-2-oxa-5-azabicyclo[2.2.1]heptan-5-yl]indazol-5-yl]-6-(trifluoromethyl)pyridine-2-carboxamide

To a solution of N-[2-[4-(hydroxymethyl)cyclohexyl]-6-[(1R,4R)-2-oxa-5-azabicyclo[2.2.1]heptan-5-yl]indazol-5-yl]-6-(trifluoromethyl)pyridine-2-carboxamide (50.0 mg, 96.9 umol) in DCM (2 mL) was added DMP (61.7 mg, 145 umol) at 0° C. The mixture was then stirred at 20° C. for 3 hrs. On completion, the mixture was quenched with saturated Na₂S₂O₃ (5 mL), and diluted with DCM (40 mL). The organic layer was washed with saturated NaHCO₃ (2×20 mL), dried over anhydrous Na₂SO₄, filtered and concentrated in vacuo to give the title compound (35.0 mg, 70% yield) as yellow solid. LC-MS (ESI⁺) m/z 514.1 (M+H)⁺.

### 6-(Trifluoromethyl)pyrazine-2-carboxamide (Intermediate BQZ)

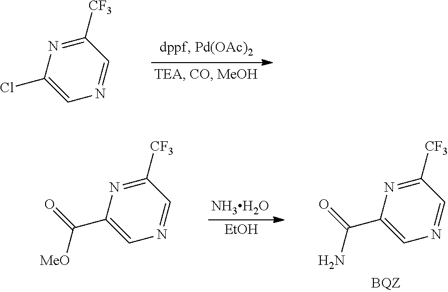

### Step 1—Methyl 6-(trifluoromethyl)pyrazine-2-carboxylate

To a solution of 2-chloro-6-(trifluoromethyl)pyrazine (1.80 g, 9.86 mmol, CAS #61655-69-4) in MeOH (20 mL) was added DPPF (273 mg, 493 umol), Pd(OAc)₂ (221 mg, 986 umol) and TEA (2.00 g, 19.7 mmol). The suspension was degassed under vacuum and purged with CO three times. The mixture was stirred under CO (50 psi) at 70° C. for 16 hours. On completion, the reaction mixture was concentrated in vacuo to give a residue. The residue was diluted with EA (30 mL) and washed with H₂O (15 mL×2). The organic layer was dried over Na₂SO₄, filtered and concentrated in vacuo to give a residue. The residue was purified by reverse phase (FA condition) to give the title compound (1.30 g, 63% yield) as light yellow solid. ¹H NMR (400 MHz, CDCl₃) δ 9.49 (s, 1H), 9.13 (s, 1H), 4.06 (s, 3H).

### Step 2—6-(Trifluoromethyl)pyrazine-2-carboxamide

To a solution of methyl 6-(trifluoromethyl)pyrazine-2-carboxylate (300 mg, 1.46 mmol) in EtOH (1 mL) was added NH₃·H₂O (3.36 mL, 21.8 mmol, 25% solution) at 25° C. and the mixture was stirred at 25° C. for 1 hr. On completion, the mixture was concentrated in vacuo to give the title compound (250 mg, 80% yield) as white solid. ¹H NMR (400 MHz, CDCl₃) δ 9.65 (s, 1H), 9.17 (s, 1H), 7.57 (s, 1H), 5.83 (s, 1H).

### ((1R,4R)-4-(5-amino-6-fluoro-2H-indazol-2-yl)cyclohexylmethanol (Intermediate BRA)

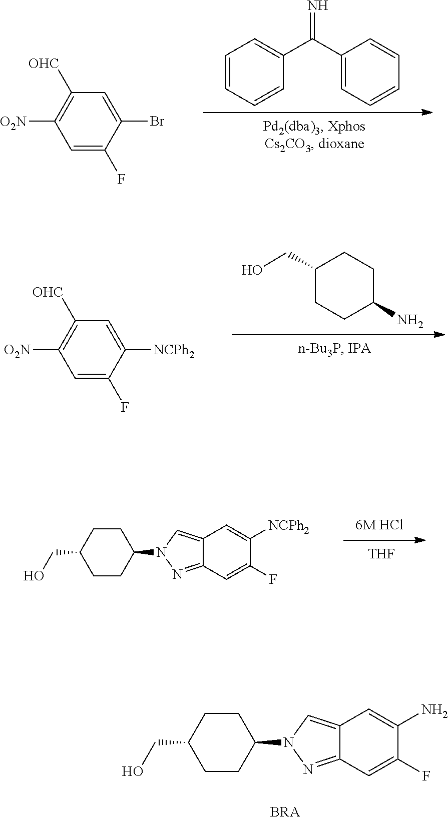

### Step 1—5-(benzhydrylideneamino)-4-fluoro-2-nitro-benzaldehyde

To a solution of 5-bromo-4-fluoro-2-nitro-benzaldehyde (1 g, 4.03 mmol, CAS #213382-45-7) and diphenylmethanimine (1.02 g, 5.65 mmol, CAS #1013-88-3) in dioxane (15 mL) was added Pd2(dba)₃ (369 mg, 403 umol), Xantphos (233 mg, 403 umol) and Cs₂CO₃ (2.63 g, 8.06 mmol). The mixture was stirred at 100° C. under N₂ for 16 hrs. On completion, the mixture was poured into water (40 mL) and the aqueous phase was extracted with ethyl acetate (2×40 mL). The combined organic phase was dried with anhydrous Na₂SO₄, filtered and concentrated in vacuo. The crude product was purified by silica gel chromatography (column height: 250 mm, diameter: 100 mm, 100-200 mesh silica gel, petroleum ether/ethyl acetate=20/1) to give the title compound (900 mg, 64% yield) as yellow solid. LC-MS (ESI⁺) m/z 348.9 (M+H)⁺.

### Step 2—((1r,4r)-4-(5-((diphenylmethylene)amino)-6-fluoro-2H-indazol-2-yl)cyclohexyl) methanol

A solution of 5-(benzhydrylideneamino)-4-fluoro-2-nitro-benzaldehyde (700 mg, 2.01 mmol) and (4-aminocyclohexyl)methanol (285 mg, 2.21 mmol, CAS #1467-84-1) in IPA (15 mL) was stirred at 80° C. for 4 hrs. The mixture was cooled to 25° C., then n-Bu₃P (1.22 g, 6.03 mmol) was added into the mixture. The reaction was stirred at 80° C. for 16 hrs under N₂. On completion, the reaction mixture was concentrated in vacuo. The residue was purified by silica gel chromatography (column height: 250 mm, diameter: 100 mm, 100-200 mesh silica gel, petroleum ether/ethyl acetate=1/1) to give the title compound (735 mg, 85% yield) as yellow solid. ¹H NMR (400 MHz, CDCl₃) δ 7.84-7.77 (m, 3H), 7.56-7.50 (m, 1H), 7.48-7.42 (m, 2H), 7.31-7.28 (m, 4H), 7.25-7.19 (m, 3H), 4.37-4.27 (m, 1H), 3.57 (d, J=6.4 Hz, 2H), 2.37-2.29 (m, 2H), 1.99-1.89 (m, 2H), 1.72-1.68 (m, 2H), 1.48-1.41 (m, 4H), 1.30-1.26 (m, 2H).

### Step 3—((1r,4r)-4-(5-amino-6-fluoro-2H-indazol-2-yl)cyclohexylmethanol

To a solution of [4-[5-(benzhydrylideneamino)-6-fluoro-indazol-2-yl]cyclohexyl]methanol (50.0 mg, 116 umol) in THF (2.5 mL) was added HCl (6 M, 3 mL) and the mixture was stirred at 20° C. for 16 hrs. On completion, the reaction mixture was concentrated in vacuo. The residue was diluted with water (10 mL) and the aqueous phase was extracted with ethyl acetate (20 mL). The aqueous phase was separated and concentrated in vacuo to give the title compound (24 mg, 77% yield). ¹H NMR (400 MHz, DMSO-d₆) δ 8.51 (s, 1H), 7.79 (d, J=8.0 Hz, 1H), 7.58 (d, J=12 Hz, 1H), 4.46-4.40 (m, J=3.9, 7.6, 11.4 Hz, 1H), 3.29 (d, J=6.1 Hz, 2H), 2.40-2.31 (m, 1H), 2.17-2.10 (m, 2H), 1.95-1.82 (m, 4H), 1.53-1.42 (m, 1H), 1.23-1.08 (m, 2H).

### N-[6-fluoro-2-(4-formylcyclohexyl)indazol-5-yl]-6-(trifluoromethyl)pyrazine-2-carboxamide (Intermediate BRB)

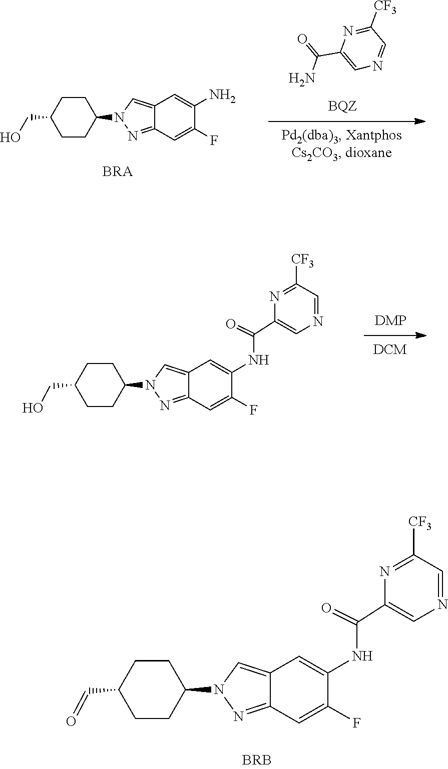 [SMILES: NC(=O)c1cncc(C(F)(F)F)n1 ; Nc1cc2cn([C@H]3CC[C@H](CO)CC3)nc2cc1F ; O=C(Nc1cc2cn([C@H]3CC[C@H](CO)CC3)nc2cc1F)c1cncc(C(F)(F)F)n1 ; O=C[C@H]1CC[C@H](n2cc3cc(NC(=O)c4cncc(C(F)(F)F)n4)c(F)cc3n2)CC1]

### Step 1—N-[6-fluoro-2-[4-(hydroxymethyl)cyclohexyl]indazol-5-yl]-6-(trifluoromethyl)pyrazine-2-carboxamide

A mixture of 6-(trifluoromethyl)pyrazine-2-carboxamide (180 mg, 942 umol, Intermediate BQZ), [4-(5-bromo-6-fluoro-indazol-2-yl)cyclohexyl]methanol (308 mg, 942 umol, Intermediate BRA), Pd₂(dba)₃ (86.2 mg, 94.2 umol), Xantphos (109 mg, 188 umol) and Cs₂CO₃ (614 mg, 1.88 mmol) in dioxane (10 mL) was degassed and purged with N₂ for three times, and then the mixture was stirred at 100° C. for 16 hrs under N₂ atmosphere. On completion, the reaction mixture was filtered and filtrate was concentrated in vacuo to give a residue. The residue was purified by column chromatography (SiO₂, petroleum ether/ethyl acetate=2/1) to give the title compound (210 mg, 51% yield) as yellow solid. ¹H NMR (400 MHz, CDCl₃) δ 9.89 (d, J=3.2 Hz, 1H), 9.74 (s, 1H), 9.20 (s, 1H), 8.81 (d, J=7.6 Hz, 1H), 8.03 (s, 1H), 7.45 (d, J=11.6 Hz, 1H), 4.43-4.33 (m, 1H), 3.60-3.56 (m, 2H), 2.39-2.33 (m, 2H), 2.10-2.03 (m, 3H), 1.33-1.23 (m, 4H); LC-MS (ESI⁺) m/z 438.1 (M+H)⁺.

### Step 2—N-[6-fluoro-2-(4-formylcyclohexyl)indazol-5-yl]-6-(trifluoromethyl)pyrazine-2-carboxamide

To a solution of N-[6-fluoro-2-[4-(hydroxymethyl)cyclohexyl]indazol-5-yl]-6-(trifluoromethyl) pyrazine-2-carboxamide (140 mg, 320 umol) in DCM (3 mL) was added DMP (176 mg, 416 umol) at 0° C. Then the mixture was stirred at 25° C. for 1 hr. On completion, the reaction mixture was quenched with saturated NaHCO₃ (1 mL) and Na₂S₂O₃ (1 mL), then extracted with DCM (5 mL). The combined organic layer was washed with NaCl (1 mL×3), dried over Na₂SO₄, filtered and concentrated in vacuo to give the title compound (134 mg, 99% yield) as yellow solid. ¹H NMR (400 MHz, CDCl₃) δ 9.90 (d, J=3.2 Hz, 1H), 9.74-9.73 (m, 1H), 8.82 (d, J=7.6 Hz, 1H), 8.00-7.95 (m, 1H), 7.45 (d, J=12.0 Hz, 1H), 7.31-7.28 (m, 1H), 4.43-4.31 (m, 1H), 2.45-2.40 (m, 3H), 2.30-2.26 (m, 2H), 2.12-2.00 (m, 2H), 1.60-1.50 (m, 2H), 1.30-1.23 (m, 1H); LC-MS (ESI⁺) m/z 436.0 (M+H)⁺.

### N-[6-fluoro-2-(4-formylcyclohexyl)indazol-5-yl]pyrazine-2-carboxamide (Intermediate BRC)

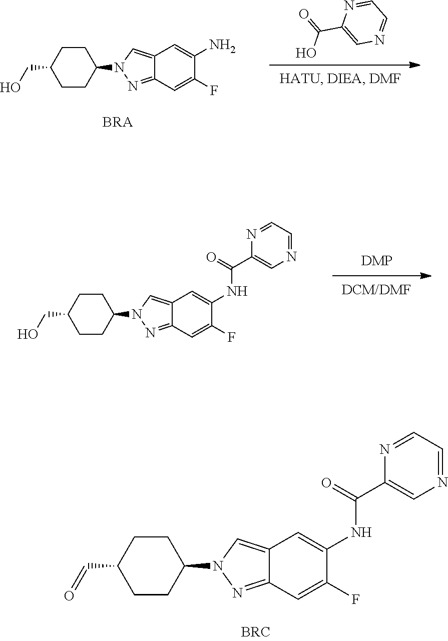 [SMILES: Nc1cc2cn([C@H]3CC[C@H](CO)CC3)nc2cc1F ; O=C(O)c1cnccn1 ; O=C(Nc1cc2cn([C@H]3CC[C@H](CO)CC3)nc2cc1F)c1cnccn1 ; O=C[C@H]1CC[C@H](n2cc3cc(NC(=O)c4cnccn4)c(F)cc3n2)CC1]

### Step 1—N-[6-fluoro-2-[4-(hydroxymethyl)cyclohexyl]indazol-5-yl]pyrazine-2-carboxamide

To a solution of [4-(5-amino-6-fluoro-indazol-2-yl)cyclohexyl]methanol (34.0 mg, 129 umol, Intermediate BRA) in DMF (2 mL) was added pyrazine-2-carboxylic acid (24.0 mg, 193 umol, CAS #98-97-5), DIEA (83.4 mg, 645 umol) and HATU (73.6 mg, 193 umol) at 0° C. Then the reaction was stirred at 0-25° C. for 1 h. On completion, the reaction mixture was diluted with EtOAc (100 mL), then washed with brine (4×40 mL). The organic layer was separated, dried over Na₂SO₄ and concentrated in vacuo. The residue was purified by reversed-phase HPLC (0.1% FA condition) to give the title compound (160 mg, 28% yield) as yellow solid. ¹H NMR (400 MHz, DMSO-d₆) δ 10.31 (d, J=1.6 Hz, 1H), 9.32 (d, J=1.6 Hz, 1H), 8.97 (d, J=2.4 Hz, 1H), 8.84 (dd, J=1.6, 2.4 Hz, 1H), 8.49 (s, 1H), 8.32 (d, J=8.0 Hz, 1H), 7.53 (d, J=12.0 Hz, 1H), 4.54-4.36 (m, 2H), 2.19-2.10 (m, 2H), 1.96-1.82 (m, 5H), 1.51-1.48 (m, 1H), 1.22-1.11 (m, 2H).

### Step 2—N-[6-fluoro-2-(4-formylcyclohexyl)indazol-5-yl]pyrazine-2-carboxamide

To a solution of N-[6-fluoro-2-[4-(hydroxymethyl)cyclohexyl]indazol-5-yl]pyrazine-2-carboxamide (20.0 mg, 54.1 umol) in DCM (1.50 mL) and DMF (0.2 mL) was added DMP (45.9 mg, 108 umol) at 0° C. Then the mixture was stirred at 0-25° C. for 3 hrs. On completion, the reaction mixture was diluted with DCM (30 mL), then quenched with Na₂S₂O₃ (aq.) (2 ml). The organic layer was washed with NaHCO₃ solution (2×20 mL), dried over Na₂SO₄ and concentrated in vacuo to give the title compound (17 mg, 85%) as a yellow solid. LC-MS (ESI⁺) m/z 368.0 (M+H)⁺.

### 4-Bromo-5-methoxy-3-methyl-1H-benzimidazol-2-one (Intermediate BRD)

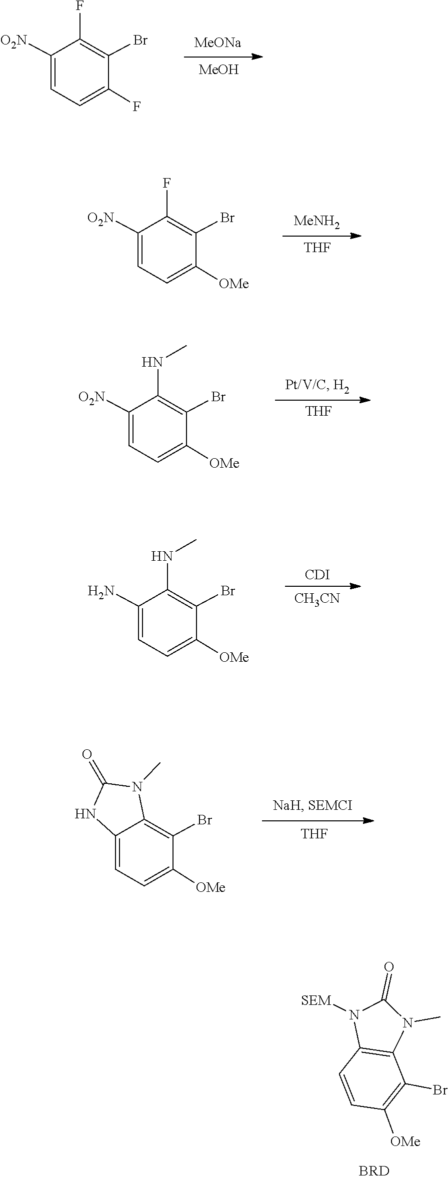

### Step 1—2-Bromo-3-fluoro-1-methoxy-4-nitro-benzene

To a mixture of 2-bromo-1,3-difluoro-4-nitro-benzene (5.00 g, 21.01 mmol, from CAS #103977-78-2) in MeOH (50 mL) was added NaOMe (1.14 g, 21.0 mmol) at 0° C. The mixture was warmed to 20° C. and stirred for 2 hours. On completion, the mixture was poured into the water (60 mL) and the aqueous phase was extracted with ethyl acetate (2×40 mL). The combined organic phase was washed with brine (2×40 mL), dried with anhydrous Na₂SO₄, filtered and concentrated in vacuo to give the crude product. The crude product was triturated with PE:EA=20:1 (10 mL) to give the title compound (1.80 g, 34% yield) as white solid. ¹H NMR (400 MHz, CDCl₃) δ 8.14 (dd, J=8.4, 9.2 Hz, 1H), 6.81 (dd, J=1.6, 9.2 Hz, 1H), 4.04 (s, 3H).

### Step 2—2-Bromo-3-methoxy-N-methyl-6-nitro-aniline

To a mixture of 2-bromo-3-fluoro-1-methoxy-4-nitro-benzene (1.60 g, 6.40 mmol) in THF (20 mL) was added MeNH₂ (2 M, 4.80 mL) and the mixture was stirred at 20° C. for 2 hours. On completion, the mixture was concentrated in vacuo to give the residue. The residue was purified by column chromatography (SiO₂, petroleum ether/ethyl acetate=50:1 to 10:1) to give the title compound (1.40 g, 83% yield) as brown solid. ¹H NMR (400 MHz, CDCl₃) δ 8.06 (d, J=9.6 Hz, 1H), 6.73 (s, 1H), 6.44 (d, J=9.2 Hz, 1H), 3.97 (s, 3H), 3.09 (d, J=5.2 Hz, 3H).

### Step 3—3-Bromo-4-methoxy-N2-methyl-benzene-1,2-diamine

To a mixture of 2-bromo-3-methoxy-N-methyl-6-nitro-aniline (1.40 g, 5.36 mmol) in THF (10 mL) and MeOH (10 mL) was added platinum (104 mg, 53.6 umol, 10 wt %) under H₂ (15 psi) and stirred at 20° C. for 2 hours. On completion, the mixture was filtered and concentrated to give the title compound (1.20 g, 96% yield) as a yellow solid.

### Step 4—4-Bromo-5-methoxy-3-methyl-1H-benzimidazol-2-one

To a mixture 3-bromo-4-methoxy-N2-methyl-benzene-1,2-diamine (1.1 g, 4.76 mmol) in CH₃CN (20 mL) was added CDI (1.16 g, 7.14 mmol) and the mixture was stirred at 90° C. for 3 hours. On completion, the mixture was concentrated to remove the CH₃CN, then H₂O (10 mL) was added into the mixture. The mixture was filtered to give the filter cake which was dried to give the title compound (1.00 g, 81% yield) as brown solid. ¹H NMR (400 MHz, CDCl₃) δ 6.89 (d, J=8.4 Hz, 1H), 6.59 (d, J=8.8 Hz, 1H), 3.81 (s, 3H), 3.71 (s, 3H), 2.72 (d, J=2.4 Hz, 1H).

### Step 5—4-Bromo-5-methoxy-3-methyl-1-((2-(trimethylsilyl)ethoxy)methyl)-1H-benzo[d]imidazol-2(3H)-one

To a solution of 7-bromo-6-methoxy-1-methyl-1H-benzo[d]imidazol-2(3H)-one (1.00 g, 3.90 mmol) in THF (30 mL) was added NaH (234 mg, 5.85 mmol, 60% dispersion in mineral oil) at 0° C. and the mixture was stirred at 0° C. for 0.5 hr. Then SEM-Cl (0.98 g, 5.85 mmol) was added to above solution and the mixture was stirred at 65° C. for 10 hrs. On completion, the mixture was quenched with H₂O (30 mL), and extracted with EA (2×30 mL). The organic layers were washed with brine (2×30 mL), dried over anhydrous Na₂SO₄, filtered and concentrated in vacuo. The mixture was purified by silica gel column (PE:EA=20:1) to give the title compound (1.30 g, 86% yield) as yellow oil. LC-MS (ESI⁺) m/z 387.0 (M+H)⁺.

### 3-(5-methoxy-3-methyl-2-oxo-4-(piperidin-4-yl)-2,3-dihydro-1H-benzo[d]imidazol-1-yl) piperidine-2,6-dione (Intermediate BRE)

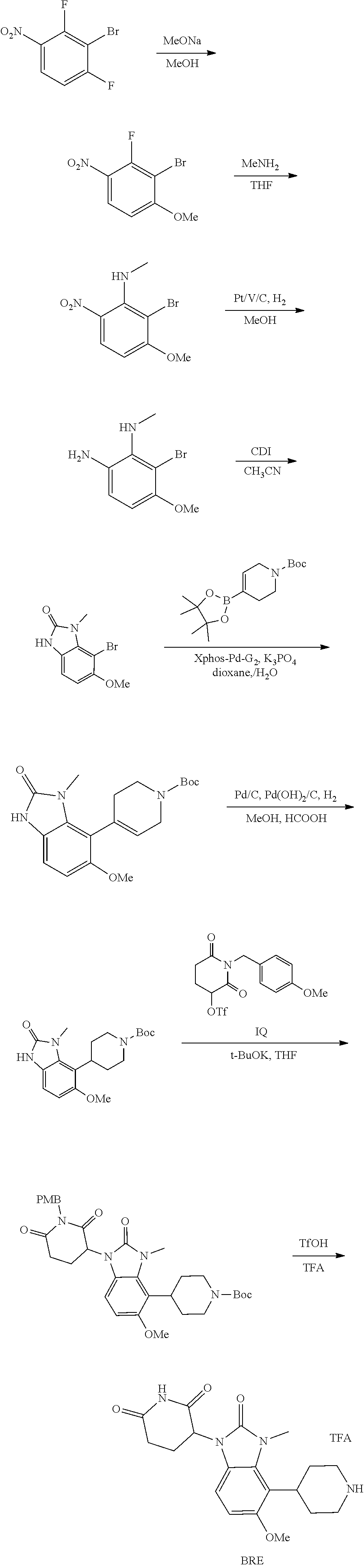

### Step 1—2-bromo-3-fluoro-1-methoxy-4-nitrobenzene

To a solution of 2-bromo-1, 3-difluoro-4-nitro-benzene (1 g, 4.20 mmol, from CAS #103977-78-2) in MeOH (10 mL) was added MeONa (226 mg, 4.20 mmol) at 0° C. The mixture was warmed to 20° C. and stirred for 2 hrs. On completion, the mixture was poured into the water (10 mL) and the aqueous phase was extracted with ethyl acetate (2×50 mL). The combined organic phase was separated, dried with anhydrous Na₂SO₄, filtered and the filtrate was concentrated in vacuo to give the crude product. The crude product was triturated with PE:EA=20:1 (20 mL) to give the title compound (130 mg, 12% yield) as white solid. ¹H NMR (400 MHz, CDCl₃) δ 8.13 (t, J=8.8 Hz, 1H), 6.80 (dd, J=1.6, 9.2 Hz, 1H), 4.03 (s, 3H).

### Step 2—2-bromo-3-methoxy-N-methyl-6-nitroaniline

To a solution of 2-bromo-3-fluoro-1-methoxy-4-nitro-benzene (3 g, 12.00 mmol) in THF (10 mL) was added 30% MeNH₂ (1.86 g, 18.00 mmol) in EtOH and the reaction was stirred at 25° C. for 2 hrs. On completion, the reaction was concentrated in vacuo. The residue was purified by column chromatography (SiO₂, petroleum ether/ethyl acetate=50/1 to 10/1) to give the title compound (3 g, 95% yield) as yellow solid. ¹H NMR (400 MHz, CDCl₃) δ 8.08 (d, J=9.2 Hz, 1H), 6.45 (d, J=9.6 Hz, 1H), 3.99 (s, 3H), 3.13-3.09 (m, 3H).

### Step 3—6-bromo-5-methoxy-N1-methylbenzene-1,2-diamine

To a solution of 2-bromo-3-methoxy-N-methyl-6-nitro-aniline (100 mg, 383 umol) in MeOH (5 mL) was added Pt/V/C (30.0 mg, 10 wt %). The reaction mixture was stirred at 25° C. under H₂ (15 psi) for 2 hrs. On completion, the reaction mixture was filtered and the filtrate was concentrated in vacuo to give the title compound (80 mg, 90% yield) as brown oil. ¹H NMR (400 MHz, CDCl₃) δ 6.58 (d, J=8.6 Hz, 1H), 6.46 (d, J=8.6 Hz, 1H), 3.74 (s, 3H), 2.65 (s, 3H).

### Step 4—7-bromo-6-methoxy-1-methyl-1H-benzo[d]imidazol-2(3H)-one

To a solution of 3-bromo-4-methoxy-N2-methyl-benzene-1,2-diamine (1.7 g, 7.36 mmol) in CH₃CN (30 mL) was added CDI (1.79 g, 11.03 mmol) and the mixture was stirred at 90° C. for 3 hrs. On completion, the mixture was concentrated in vacuo to remove MeCN, then H₂O (20 mL) was added. The mixture was filtered to give the filter cake. The filter cake was triturated with PE:EA=3:1 (40 mL) to give the title compound (1.7 g, 90% yield) as yellow solid. ¹H NMR (400 MHz, DMSO-d₆) δ 10.94 (s, 1H), 6.91 (d, J=8.4 Hz, 1H), 6.75 (d, J=8.4 Hz, 1H), 3.79 (s, 3H), 3.57 (s, 3H).

### Step 5—Tert-butyl 4-(5-methoxy-3-methyl-2-oxo-2, 3-dihydro-1H-benzo[d]imidazol-4-yl)-5, 6-dihydropyridine-1(2H)-carboxylate

A solution of 4-bromo-5-methoxy-3-methyl-1H-benzimidazol-2-one (300 mg, 1.17 mmol), tert-butyl 4-(4,4,5,5-tetramethyl-1,3,2-dioxaborolan-2-yl)-3,6-dihydro-2H-pyridine-1-carboxylate (541 mg, 1.75 mmol, CAS #286961-14-6), Xphos-PD-G2 (91.8 mg, 116 umol) and K₃PO₄ (495 mg, 2.33 mmol) in dioxane (10 mL) and H₂O (1 mL) was stirred at 80° C. for 16 hrs under N₂. On completion, the reaction mixture was extracted with EtOAc (2×40 mL). The combined organic layers were washed with water (2×30 mL), dried over Na₂SO₄ and concentrated in vacuo. The residue was purified by reversed-phase HPLC (0.1% FA condition) to give the title compound (130 mg, 31% yield) as yellow solid. ¹H NMR (400 MHz, MeOD) δ 6.94 (d, J=8.4 Hz, 1H), 6.73 (d, J=8.4 Hz, 1H), 5.64 (s, 1H), 4.22-4.12 (m, 1H), 4.09-3.97 (m, 1H), 3.80-3.73 (m, 4H), 3.65-3.53 (m, 1H), 3.36 (s, 3H), 3.34-3.31 (m, 2H), 2.60 (d, J=16.8 Hz, 1H), 2.29-2.19 (m, 1H), 1.52 (s, 10H).

### Step 6—Tert-butyl 4-(5-methoxy-3-methyl-2-oxo-2,3-dihydro-1H-benzo[d]imidazol-4-yl)piperidine-1-carboxylate

To a solution of tert-butyl 4-(5-methoxy-3-methyl-2-oxo-1H-benzimidazol-4-yl)-3,6-dihydro-2H-pyridine-1-carboxylate (100 mg, 278 umol) in HCOOH (0.05 mL) and MeOH (50 mL) was added Pd/C (50 mg, 10 wt %) and Pd(OH)₂/C(50 mg, 10 wt %). The reaction mixture was stirred at 60° C. for 48 hrs under H₂ (50 psi). On completion, the reaction mixture was filtered and the filtrate was concentrated in vacuo to give the title compound (80 mg, 79% yield) as white solid. ¹H NMR (400 MHz, CDCl₃) δ 6.89 (d, J=8.4 Hz, 1H), 6.74 (d, J=8.4 Hz, 1H), 4.19 (d, J=13.2 Hz, 2H), 3.80 (s, 3H), 3.65 (s, 3H), 3.62-3.51 (m, 2H), 2.90 (s, 1H), 2.48-2.41 (m, 2H), 1.97-1.84 (m, 2H), 1.52 (s, 9H).

### Step 7—Tert-butyl 4-(5-methoxy-1-(1-(4-methoxybenzyl)-2,6-dioxopiperidin-3-yl)-3-methyl-2-oxo-2, 3-dihydro-1H-benzo[d]imidazol-4-yl)piperidine-1-carboxylate

To a solution of tert-butyl 4-(5-methoxy-3-methyl-2-oxo-1H-benzimidazol-4-yl)piperidine-1-carboxylate (70.0 mg, 193 umol) and t-BuOK (43.4 mg, 387 umol) in THF (5 mL) was added a solution of [1-[(4-methoxyphenyl)methyl]-2,6-dioxo-3-piperidyl]trifluoromethanesulfonate (147 mg, 387 umol, Intermediate IQ) in THF(0.5 mL) at 0° C. The reaction mixture was stirred at 0° C. for 2 h. On completion, the reaction was quenched with saturated NH₄Cl solution (0.5 mL), then diluted with EtOAc (50 mL). The organic layer was washed with brine (2×20 mL), dried over Na₂SO₄ and concentrated in vacuo. The residue was purified by prep-TLC (PE:EA=3:2, Rf=0.3) to give the title compound (70.0 mg, 60% yield). ¹H NMR (400 MHz, CDCl₃) δ 7.30 (d, J=8.8 Hz, 2H), 6.75 (d, J=7.6 Hz, 2H), 6.40 (d, J=8.8 Hz, 1H), 6.19 (d, J=8.8 Hz, 1H), 5.15-5.05 (m, 1H), 4.89 (s, 2H), 4.20-4.14 (m, 1H), 3.72 (s, 3H), 3.69 (s, 3H), 3.59 (s, 3H), 3.39-3.30 (m, 1H), 2.97-2.89 (m, 1H), 2.86-2.66 (m, 4H), 2.62-2.47 (m, 2H), 2.41-2.22 (m, 3H), 2.11-2.03 (m, 1H), 1.43 (s, 9H).

### Step 8—3-(5-methoxy-3-methyl-2-oxo-4-(piperidin-4-yl)-2,3-dihydro-1H-benzo[d]imidazol-1-yl) piperidine-2,6-dione

A solution of tert-butyl 4-[5-methoxy-1-[1-[(4-methoxyphenyl) methyl]-2, 6-dioxo-3-piperidyl]-3-methyl-2-oxo-benzimidazol-4-yl]piperidine-1-carboxylate (60 mg, 101 umol) in TFA (1 mL) and TfOH (0.2 mL) was stirred at 70° C. for 1h. On completion, the reaction mixture was concentrated in vacuo. The residue was purified by prep-HPLC (column: Phenomenex Luna C18 150*25 mm*10 um; mobile phase: [water (0.1% TFA) ACN]; B %: 2%-32%, 10 min) to give the title compound (25 mg, 66% yield) as yellow solid. LC-MS (ESI⁺) m/z 373.0 (M+H)⁺.

### 3-(4-(Azetidin-3-yl)-3-methyl-2-oxo-2,3-dihydro-1H-benzo[d]imidazol-1-yl)piperidine-2,6-dione (Intermediate BRF)

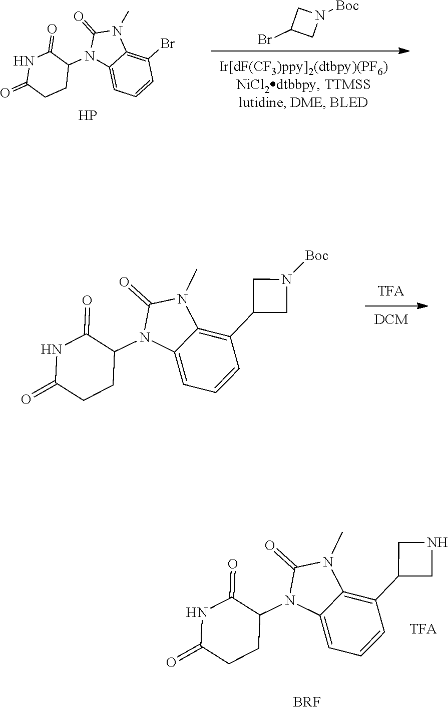

### Step 1—Tert-butyl 3-(1-(2,6-dioxopiperidin-3-yl)-3-methyl-2-oxo-2,3-dihydro-1H-benzo[d]imidazol-4-yl)azetidine-1-carboxylate

To a 40 mL vial equipped with a stir bar was added 3-(4-bromo-3-methyl-2-oxo-benzimidazol-1-yl)piperidine-2,6-dione (1.0 g, 2.96 mmol, Intermediate HP), tert-butyl 3-iodoazetidine-1-carboxylate (1.09 g, 3.84 mmol, CAS #254454-54-1), Ir[dF(CF3)ppy]2(dtbpy)(PF6) (33.1 mg, 29.5 umol), TTMSS (735 mg, 2.96 mmol), 2,6-dimethylpyridine (633 mg, 5.91 mmol) and NiCl₂-dtbbpy (5.88 mg, 14.7 umol) in DME (24 mL). The reaction was stirred and irradiated with a 34 W blue LED lamp at 25° C. for 14 hr. On completion, the mixture was filtered and the filtrate was concentrated in vacuo. The crude product was purified by reverse phase flash (0.1% FA condition) to give the title compound (900 mg, 71% yield) as white solid. 1H NMR (400 MHz, DMSO-d6) δ 11.10 (s, 1H), 7.24 (d, J=4.4 Hz, 1H), 7.14-7.00 (m, 2H), 5.42-5.34 (m, 1H), 4.56-4.46 (m, 1H), 4.29 (t, J=8.4 Hz, 2H), 3.97 (t, J=6.0 Hz, 2H), 3.51 (s, 3H), 2.96-2.81 (m, 1H), 2.78-2.59 (m, 2H), 2.06-1.95 (m, 1H), 1.42 (s, 9H).

### Step 2—3-(4-(Azetidin-3-yl)-3-methyl-2-oxo-2,3-dihydro-1H-benzo[d]imidazol-1-yl)piperidine-2,6-dione

To a solution of tert-butyl 3-[1-(2,6-dioxo-3-piperidyl)-3-methyl-2-oxo-benzimidazol-4-yl]azetidine-1-carboxylate (150 mg, 361 umol) in DCM (3.0 mL) was added TFA (1.54 g, 13.5 mmol). The mixture was stirred at 25° C. for 30 min. On completion, the reaction mixture was concentrated in vacuo to give the title compound (120 mg, 80% yield, TFA) as brown solid. LC-MS (ESI⁺) m/z 315.2 (M+H)⁺.

### 3-(4-Bromo-3,5-dimethyl-2-oxo-2,3-dihydro-1H-benzo[d]imidazol-1-yl)piperidine-2,6-dione (Intermediate BRG)

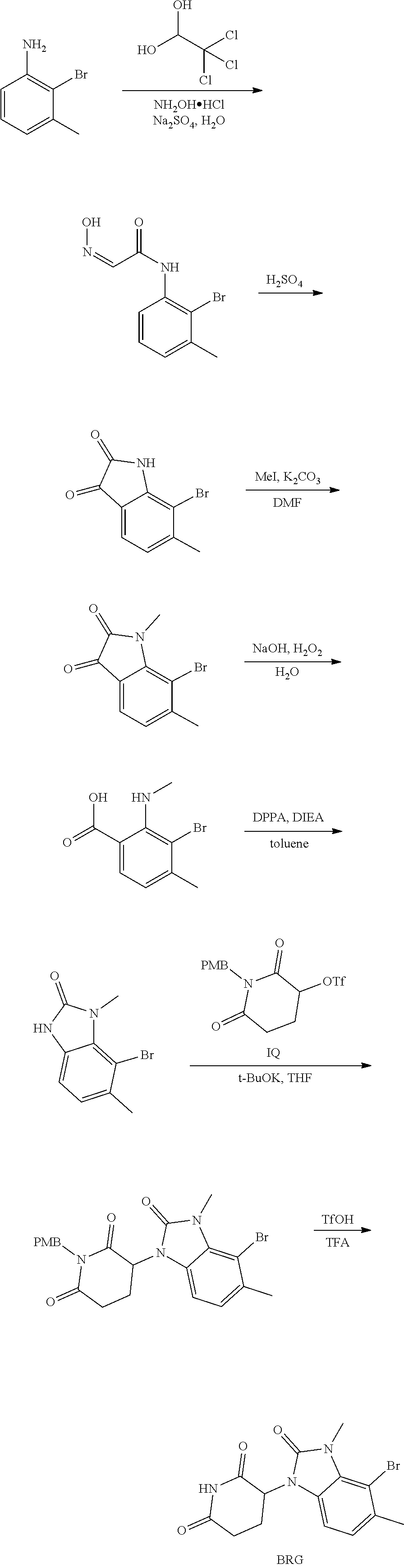

### Step 1—(Z)—N-(2-bromo-3-methylphenyl)-2-(hydroxyimino)acetamide

To a mixture of 2-bromo-3-methyl-aniline (12.0 g, 64.5 mmol, CAS #54879-20-8) and 2,2,2-trichloroethane-1,1-diol (16.0 g, 96.7 mmol, CAS #302-17-0) in H₂O (250 mL) was added Na₂SO₄ (100 g, 709 mmol) and NH₂OH—HCl (6.27 g, 90.3 mmol) at 25° C. under N₂. The mixture was stirred at 25° C. for 16 hours. Then, HCl (12 M, 18.8 mL) was added, and the mixture was stirred at 100° C. for 16 hours. On completion, the reaction mixture was cooled to 0° C. and filtered. The filter cake was dried in vacuo to give the title compound (14.0 g, 84% yield) as a yellow solid. LC-MS (ESI⁺) m/z 256.9 (M+H)⁺.

### Step 2—7-Bromo-6-methylindoline-2,3-dione

A mixture of (1E)-2-(2-amino-3-bromo-4-methyl-phenyl)-2-oxo-acetaldehyde oxime (14.0 g, 54.4 mmol) in H₂SO₄ (200 mL) was stirred at 80° C. for 3 hours. On completion, the reaction mixture was poured into ice water (1000 mL), and filtered. The filtered cake was collected and dried in vacuo to give the title compound (12 g, 92% yield) as a yellow solid. LC-MS (ESI⁺) m/z 239.9 (M+H)⁺.

### Step 3—7-Bromo-1,4-dimethyl-indoline-2,3-dione

To a solution of 7-bromo-6-methyl-indoline-2,3-dione (2.00 g, 8.33 mmol) in the DMF (30 mL) was added K₂CO₃ (3.45 g, 25.0 mmol) and MeI (3.55 g, 25.0 mmol, 1.56 mL) and the mixture was stirred at 60° C. for 1 hr. On completion, the reaction was quenched with water (20 mL) and extracted with EA (100 mL). The organic layer was dried over Na₂SO₄, filtered and concentrated in vacuo. The residue was purified by column chromatography (petroleum ether/ethyl acetate=20/1 to 5/1) to give the title compound (800 mg, 38% yield) as a red solid. ¹H NMR (400 MHz, CDCl₃) δ 7.47 (d, J=7.6 Hz, 1H), 7.03 (d, J=7.6 Hz, 1H), 3.69 (s, 3H), 2.50 (s, 3H).

### Step 4—3-Bromo-4-methyl-2-(methylamino)benzoic acid

To a solution of 7-bromo-1,6-dimethyl-indoline-2,3-dione (800 mg, 3.15 mmol) in the 2 M aqueous NaOH (1.26 g, 31.4 mmol) in H₂O (15 mL) was added H₂O₂ (1.43 g, 12.6 mmol, 1.21 mL, 30% solution) at 0° C. in dropwise. The mixture was stirred at 20° C. for 2 hrs. On completion, the reaction mixture was acidified with HCl (6 M) to pH=5˜6 and the mixture was extracted with EA (100 mL). The organic layer was dried over Na₂SO₄, filtered and concentrated in vacuo to give the title compound (630 mg, 82% yield) as a yellow solid. ¹H NMR (400 MHz, DMSO-d₆) δ 7.64 (d, J=8.0 Hz, 1H), 6.84 (d, J=8.0 Hz, 1H), 2.87 (s, 3H), 2.36 (s, 3H).

### Step 5—7-Bromo-1,6-dimethyl-1H-benzo[d]imidazol-2(3H)-one

To a solution of 3-bromo-4-methyl-2-(methylamino)benzoic acid (400 mg, 1.64 mmol) in the t-BuOH (3 mL) was added DIEA (635 mg, 4.92 mmol) and DPPA (676 mg, 2.46 mmol). The mixture was stirred at 95° C. for 12 hrs under N₂. On completion, the mixture was concentrated in vacuo. The residue was diluted with toluene (3 mL), filtered and the filtered cake was collected and dried in vacuo to give the title compound (360 mg, 91% yield) as a gray solid. ¹H NMR (400 MHz, DMSO-d₆) δ 11.06 (s, 1H), 6.97 (d, J=8.0 Hz, 1H), 6.88 (d, J=8.0 Hz, 1H), 3.59 (s, 3H), 2.34 (s, 3H).

### Step 6—3-(4-Bromo-3,5-dimethyl-2-oxo-2,3-dihydro-1H-benzo[d]imidazol-1-yl)-1-(4-methoxybenzyl) piperidine-2,6-dione

To a solution of 4-bromo-3,5-dimethyl-1H-benzimidazol-2-one (360 mg, 1.49 mmol) in the THF (4 mL) was added tBuOK (251 mg, 2.24 mmol) at −10° C. Then [1-[(4-methoxyphenyl)methyl]-2,6-dioxo-3-piperidyl]trifluoromethanesulfonate (854 mg, 2.24 mmol, Intermediate IQ) in the THF (4 mL) was added at −10° C. and the mixture was stirred for 1 hr. On completion, the reaction mixture was acidified with FA to pH=5˜6. Then the mixture was diluted with EA (30 mL) and washed with water (2×30 mL). The organic layer was dried over Na₂SO₄, filtered and concentrated in vacuo. The residue was purified by column chromatography (petroleum ether/ethyl acetate=10/1 to 2/1) to give the title compound (650 mg, 92% yield) as a white solid. LC-MS (ESI⁺) m/z 472.1 (M+H)⁺.

### Step 7—3-(4-Bromo-3,5-dimethyl-2-oxo-2,3-dihydro-1H-benzo[d]imidazol-1-yl)piperidine-2,6-dione

A solution of 3-(4-bromo-3,5-dimethyl-2-oxo-benzimidazol-1-yl)-1-[(4-methoxyphenyl)methyl]piperidine-2,6-dione (100 mg, 211 umol) in the TFA (1 mL) and TfOH (0.2 mL) was stirred at 70° C. for 3 hrs. On completion, the reaction mixture was concentrated by blowing with a steam N₂. The residue was purified by reversed-phase (0.1% FA condition) to give the title compound (55 mg, 74% yield) as a white solid. ¹H NMR (400 MHz, DMSO-d₆) δ 11.12 (s, 1H), 7.11-7.03 (m, 2H), 5.39 (dd, J=5.6, 12.6 Hz, 1H), 3.67 (s, 3H), 2.94-2.83 (m, 1H), 2.76-2.68 (m, 1H), 2.66-2.59 (m, 1H), 2.38 (s, 3H), 2.07-1.97 (m, 1H), LC-MS (ESI⁺) m/z 352.2 (M+H)⁺.

### Tert-butyl-[(4-ethynylcyclohexyl)methoxy]-dimethyl-silane (Intermediate BRH)

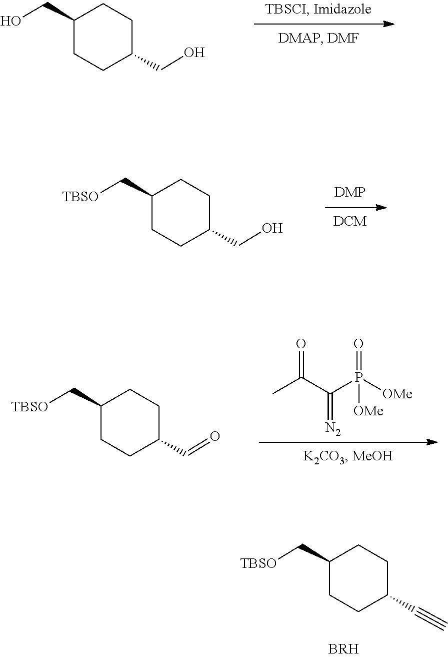

### Step 1—[4-[[Tert-butyl(dimethyl)silyl]oxymethyl]cyclohexyl]methanol

To a mixture of [4-(hydroxymethyl)cyclohexyl]methanol (6.00 g, 41.6 mmol, CAS #3236-48-4), imidazole (5.67 g, 83.2 mmol) and DMAP (508 mg, 4.16 mmol) in DMF (70 mL) was added TBSCI (6.27 g, 41.6 mmol, 5.10 mL) slowly at 0° C. The reaction mixture was stirred at 25° C. for 12 hours. On completion, the reaction mixture was quenched with water (50 mL) and extracted with EA (3×60 mL). The combined organic layer was dried over Na₂SO₄, filtered and concentrated in vacuo to give a residue. The residue was purified by column chromatography to give the title compound (4.50 g, 410% yield) as colorless oil. ¹H NMR (400 MHz, CDCl₃) δ 3.46 (d, J=6.4 Hz, 2H), 3.41 (d, J=6.4 Hz, 2H), 1.88-1.75 (m, 4H), 1.51-1.40 (m, 2H), 1.37 (s, 1H), 1.02-0.91 (m, 4H), 0.90 (s, 9H), 0.07-0.01 (m, 6H).

### Step 2—4-[[Tert-butyl(dimethyl)silyl]oxymethyl]cyclohexanecarbaldehyde

To a mixture of [4-[[tert-butyl(dimethyl)silyl]oxymethyl]cyclohexyl]methanol (4.50 g, 17.4 mmol) in DCM (50 mL) was added DMP (8.86 g, 20.8 mmol, 6.47 mL) at 0° C. dropwise. The reaction mixture was then stirred at 25° C. for 3 hr. On completion, the reaction mixture was quenched with saturated Na₂S₂O₃ (20 mL) and saturated NaHCO₃ (20 mL) at 25° C., and then stirred for 30 minutes. The mixture was then extracted with DCM (3×50 mL). Then the organic layers were separated and concentrated in vacuo to give the crude product. The residue was purified by column chromatography to give the title compound (3.00 g, 67% yield) as a light yellow oil. ¹H NMR (400 MHz, CDCl3) δ 9.62 (d, J=1.6 Hz, 1H), 3.43 (d, J=6.0 Hz, 2H), 2.23-2.12 (m, 1H), 2.06-1.96 (m, 2H), 1.93-1.85 (m, 2H), 1.51-1.37 (m, 1H), 1.35-1.22 (m, 2H), 1.06-0.94 (m, 2H), 0.92-0.89 (m, 9H), 0.06-0.03 (m, 6H).

### Step 3—Tert-butyl-[(4-ethynylcyclohexyl)methoxy]-dimethyl-silane

To a mixture of 4-[[tert-butyl(dimethyl)silyl]oxymethyl]cyclohexanecarbaldehyde (3.00 g, 11.7 mmol) and K₂CO₃ (3.23 g, 23.4 mmol) in MeOH (30 mL) was added 1-diazo-1-dimethoxyphosphoryl-propan-2-one (3.37 g, 17.5 mmol, CAS #90965-06-3) at 0° C. dropwise. The reaction mixture was then stirred at 25° C. for 12 hours. On completion, the reaction mixture was diluted with water (50 mL) and extracted with EA (3×30 mL). The combined organic layer was dried over Na₂SO₄, filtered and concentrated in vacuo to give a residue. The residue was purified by column chromatography to give the title compound (2.60 g, 88% yield) as colorless oil. ¹H NMR (400 MHz, CDCl₃) δ 3.39 (d, J=6.4 Hz, 2H), 2.25-2.13 (m, 1H), 2.04 (d, J=2.4 Hz, 1H), 2.04-1.98 (m, 2H), 1.84-1.75 (m, 2H), 1.51-1.44 (m, 1H), 1.43-1.32 (m, 2H), 0.93 (d, J=3.2 Hz, 2H), 0.89 (s, 9H), 0.04 (s, 6H).

### 3-[5-Chloro-3-methyl-4-[4-(methylamino)-1-piperidyl]-2-oxo-benzimidazol-1-yl]piperidine-2,6-dione (Intermediate BOK)

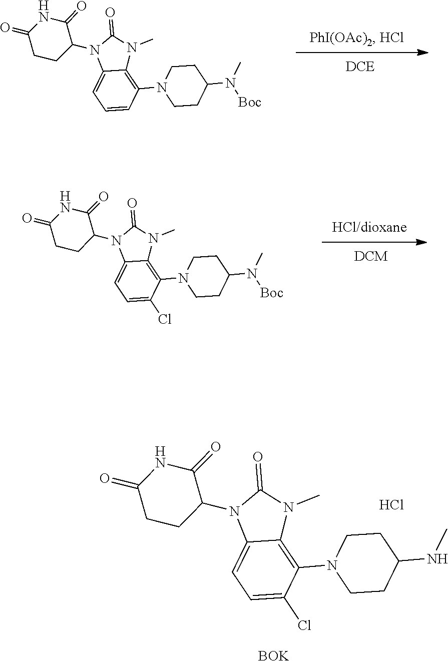 [SMILES: CN(C(=O)OC(C)(C)C)C1CCN(c2cccc3c2n(C)c(=O)n3C2CCC(=O)NC2=O)CC1 ; CN(C(=O)OC(C)(C)C)C1CCN(c2c(Cl)ccc3c2n(C)c(=O)n3C2CCC(=O)NC2=O)CC1 ; CNC1CCN(c2c(Cl)ccc3c2n(C)c(=O)n3C2CCC(=O)NC2=O)CC1 ; Cl ; "BOK"]

### Step 1—Tert-butyl N-[1-[5-chloro-1-(2,6-dioxo-3-piperidyl)-3-methyl-2-oxo-benzimidazol-4-yl]-4-piperidyl]-N-methylcarbamate

To a solution of tert-butyl N-[1-[1-(2,6-dioxo-3-piperidyl)-3-methyl-2-oxo-benzimidazol-4-yl]-4-piperidyl]-N-methyl-carbamate (50.0 mg, 106 umol, synthesized via Step 1 of Intermediate AQK) in DCE (5.00 mL) was added PhI(OAc)₂ (34.2 mg, 106 umol) and HCl (1 M, 530.17 uL). Then the mixture was stirred at 50° C. for 16 hrs. On completion, the reaction mixture was washed with saturated solution of NaHCO₃ (1×10 mL), and then washed with saturated solution of Na₂S₂O₃ (1×10 mL). The organic layer was washed with brine (2×10 mL), then dried over anhydrous Na₂SO₄, filtered and concentrated in vacuo to give a residue. The residue was purified by prep-TLC (SiO₂, EA:PE=2:1, Rf=0.35) to give the title compound (10.0 mg, 18% yield) as a white solid. ¹H NMR (400 MHz, CDCl₃) δ 8.46 (s, 1H), 6.97 (d, J=8.4 Hz, 1H), 6.57 (d, J=8.4 Hz, 1H), 5.20 (dd, J=5.6, 12.6 Hz, 1H), 3.77 (s, 3H), 3.76-3.72 (m, 1H), 3.05-2.97 (m, 2H), 2.96-2.89 (m, 1H), 2.87-2.77 (m, 4H), 2.75-2.63 (m, 1H), 2.24-2.17 (m, 1H), 1.93-1.81 (m, 2H), 1.74-1.67 (m, 4H), 1.49 (s, 9H); LC-MS (ESI⁺) m/z 506.1 (M+H)⁺.

### Step 2—3-[5-Chloro-3-methyl-4-[4-(methylamino)-1-piperidyl]-2-oxo-benzimidazol-1-yl]piperidine-2,6-dione

To a solution of tert-butyl N-[1-[5-chloro-1-(2,6-dioxo-3-piperidyl)-3-methyl-2-oxo-benzimidazol-4-yl]-4-piperidyl]-N-methylcarbamate (10.0 mg, 19.7 umol) in DCM (0.50 mL) was added HCl/dioxane (4 M, 0.20 mL) and the mixture was stirred at 25° C. for 1 hr. On completion, the reaction mixture was concentrated in vacuo to give the title compound (8.00 mg, 910% yield, HCl) as a white solid. LC-MS (ESI⁺) m/z 406.1 (M+H)⁺.

### 3-(6-Chloro-3-methyl-4-(4-(methylamino)piperidin-1-yl)-2-oxo-2,3-dihydro-1H-benzo[d]imidazol-1-yl)piperidine-2,6-dione (Intermediate BQQ)

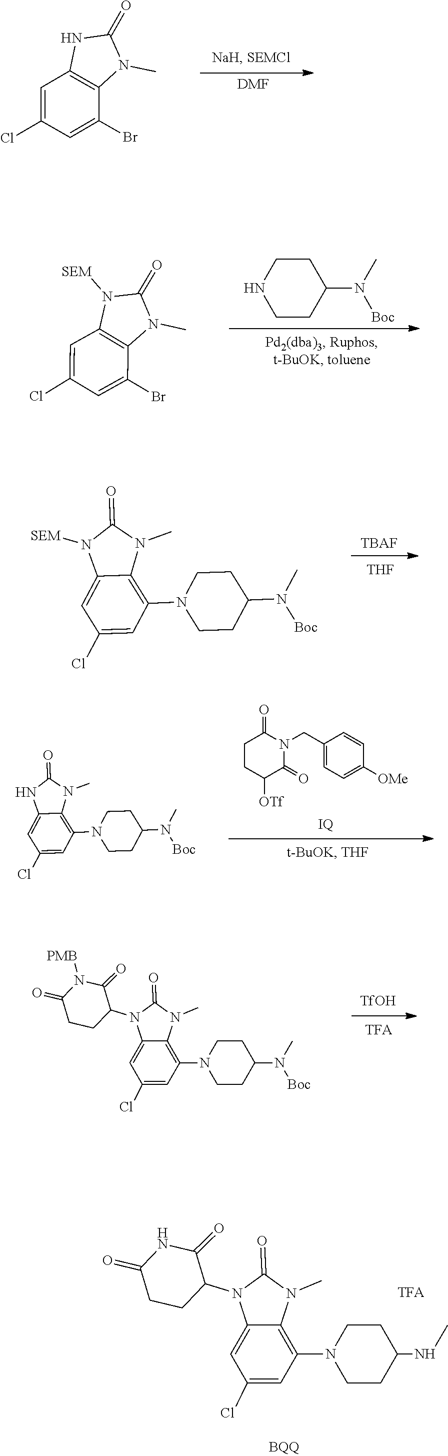

### Step 1—4-Bromo-6-chloro-3-methyl-1-((2-(trimethylsilyl)ethoxy)methyl)-1H-benzo[d]imidazol-2(3H)-one

To a solution of 4-bromo-6-chloro-3-methyl-1H-benzimidazol-2-one (3 g, 11.5 mmol, synthesized via Steps 1-4 of Intermediate BQN) in the DMF (40 mL) was added NaH (688 mg, 17.2 mmol, 60% dispersion in mineral oil) at 0° C. and the mixture was stirred for 30 mins. Then SEM-Cl (2.87 g, 17.2 mmol) was added at 0° C. and the mixture was stirred at 20° C. for 12 hrs. On completion, the reaction mixture was quenched by water (100 mL) and extracted with EA (200 mL). The organic layer was dried over Na₂SO₄, filtered and concentrated in vacuo. The residue was purified by column chromatography (PE:EA=50:1 to 20:1) to give the title compound (3.6 g, 80% yield) as white solid. ¹H NMR (400 MHz, CDCl₃) δ 7.25 (d, J=2.0 Hz, 1H), 7.12 (d, J=2.0 Hz, 1H), 5.28 (s, 2H), 3.74 (s, 3H), 3.62-3.56 (m, 2H), 0.97-0.89 (m, 2H), 0.00 (s, 9H).

### Step 2—Tert-butyl (1-(6-chloro-3-methyl-2-oxo-1-((2-(trimethylsilyl)ethoxy)methyl)-2,3-dihydro-1H-benzo[d]imidazol-4-yl)piperidin-4-yl)(methyl)carbamate

A mixture of 4-bromo-6-chloro-3-methyl-1-(2-trimethylsilylethoxymethyl)benzimidazol-2-one (1 g, 2.55 mmol), tert-butyl N-methyl-N-(4-piperidyl)carbamate (656 mg, 3.06 mmol, CAS #108612-54-0), Pd₂(dba)₃ (234 mg, 255.26 umol), RuPhos (238 mg, 510 umol) and tBuONa (613 mg, 6.38 mmol) in the toluene (20 mL) was stirred at 60° C. for 1 hr under N₂. On completion, the reaction mixture was diluted with EA (200 mL) and washed with water (2×100 mL). The organic layer was dried over Na₂SO₄, filtered and concentrated in vacuo. Then the residue was purified by column chromatography (petroleum ether/wthyl acetate=20/1 to 3/1) to give the title compound (1.2 g, 90% yield) as yellow oil. ¹H NMR (400 MHz, CDCl₃) δ 6.96 (d, J=2.0 Hz, 1H), 6.88 (d, J=1.6 Hz, 1H), 5.26 (s, 2H), 3.72 (s, 3H), 3.63-3.58 (m, 2H), 3.20 (d, J=11.8 Hz, 2H), 2.88-2.75 (m, 5H), 1.96-1.83 (m, 2H), 1.81-1.74 (m, 2H), 1.49 (s, 9H), 0.96-0.91 (m, 2H), 0.00 (s, 9H).

### Step 3—Tert-butyl (1-(6-chloro-3-methyl-2-oxo-2,3-dihydro-1H-benzo[d]imidazol-4-yl)piperidin-4-yl) (methyl)carbamate

To a solution of tert-butyl N-[1-[6-chloro-3-methyl-2-oxo-1-(2-trimethylsilylethoxymethyl) benzimidazol-4-yl]-4-piperidyl]-N-methyl-carbamate (1.2 g, 2.29 mmol) in the THF (6 mL) was added TBAF (5.97 g, 22.8 mmol). The mixture was then stirred at 80° C. for 12 hrs. On completion, the reaction mixture was filtered and concentrated in vacuo. The residue was purified by column chromatography (petroleum ether/ethyl acetate=10/1 to 1/1) to give the title compound (800 mg, 89% yield) as white solid. ¹H NMR (400 MHz, CDCl₃) δ 10.23 (s, 1H), 6.89 (d, J=1.6 Hz, 1H), 6.82 (s, 1H), 3.72 (s, 3H), 3.19 (d, J=11.6 Hz, 2H), 2.85-2.76 (m, 5H), 1.95-1.85 (m, 2H), 1.80-1.74 (m, 2H), 0.89-0.84 (m, 1H).

### Step 4—Tert-butyl (1-(6-chloro-1-(1-(4-methoxybenzyl)-2,6-dioxopiperidin-3-yl)-3-methyl-2-oxo-2,3-dihydro-1H-benzo[d]imidazol-4-yl)piperidin-4-yl)(methyl)carbamate

To a solution of tert-butyl N-[1-(6-chloro-3-methyl-2-oxo-1H-benzimidazol-4-yl)-4-piperidyl]-N-methyl-carbamate (400 mg, 1.01 mmol) in the THF (5 mL) was added tBuOK (170.49 mg, 1.52 mmol) at −10° C. and the mixture was stirred for 30 mins. Then a solution of [1-[(4-methoxyphenyl)methyl]-2,6-dioxo-3-piperidyl]trifluoromethanesulfonate (463 mg, 1.22 mmol, Intermediate IQ) in the THF (3 mL) was added dropwise in the mixture at −10° C. The mixture was stirred for 1 hr at −10° C. On completion, the reaction mixture was acidified with FA to pH=5˜6. Then the mixture was diluted with EA (100 mL), washed with NH₄Cl (100 mL) and brine (100 mL). The organic layer was dried over Na₂SO₄, filtered and concentrated in vacuo. The residue was purified by column chromatography (petroleum ether/ethyl acetate=10/1 to 1/1) to give the title compound (600 mg, 95% yield) as yellow solid. ¹H NMR (400 MHz, CDCl₃) δ 7.36 (d, J=8.8 Hz, 2H), 6.85 (d, J=8.8 Hz, 3H), 6.31 (s, 1H), 5.18 (dd, J=5.6, 13.2 Hz, 1H), 5.04-4.90 (m, 2H), 3.79 (s, 3H), 3.74 (s, 3H), 3.25-3.15 (m, 2H), 3.06-2.97 (m, 1H), 2.88-2.77 (m, 6H), 2.58 (dq, J=4.2, 13.4 Hz, 1H), 2.20-2.10 (m, 1H), 1.97-1.83 (m, 2H), 1.82-1.74 (m, 2H), 1.49 (s, 9H).

### Step 5—3-(6-Chloro-3-methyl-4-(4-(methylamino)piperidin-1-yl)-2-oxo-2,3-dihydro-1H-benzo[d]imidazol-1-yl)piperidine-2,6-dione

A mixture of tert-butyl N-[1-[6-chloro-1-[1-[(4-methoxyphenyl)methyl]-2,6-dioxo-3-piperidyl]-3-methyl-2-oxo-benzimidazol-4-yl]-4-piperidyl]-N-methyl-carbamate (200 mg, 319 umol) in the TFA (2 mL) and TfOH (0.4 mL) was stirred at 70° C. for 12 hrs. On completion, the reaction mixture was concentrated by N₂ and based with TEA to pH˜7. The residue was purified by reversed phase (0.1%, FA) to give the title compound (100 mg, 76% yield) as yellow solid. LC-MS (ESI⁺) m/z 406.2 (M+H)⁺.

### [4-[5-Bromo-6-[(1S,4S)-2-oxa-5-azabicyclo[2.2.1]heptan-5-yl]indazol-2-yl]cyclohexyl]methanol (Intermediate BAR)

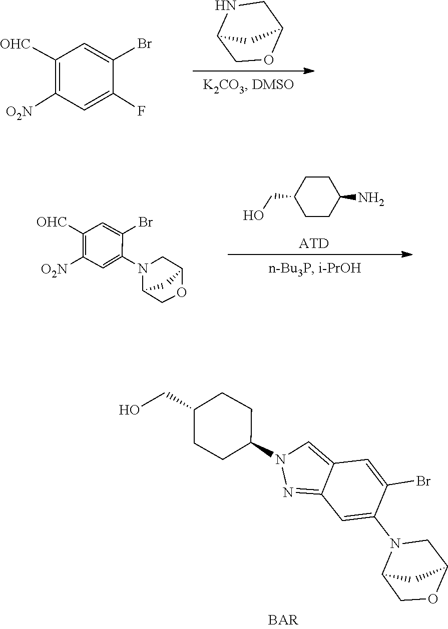

### Step 1—[4-[5-Bromo-6-[(1S,4S)-2-oxa-5-azabicyclo[2.2.1]heptan-5-yl]indazol-2-yl]cyclohexyl] methanol

To a solution of (1S,4S)-2-oxa-5-azabicyclo[2.2.1]heptane (4.00 g, 40.3 mmol, CAS #279-33-4), K₂CO₃ (5.57 g, 40.3 mmol) in DMSO (60 mL) was added with 5-bromo-4-fluoro-2-nitro-benzaldehyde (5.00 g, 20.2 mmol, synthesized via Step 1 of Intermediate ATE). The reaction mixture was stirred at 80° C. for 1 hours. On completion, the reaction mixture was diluted with EtOAc (50 mL) and extracted with EtOAc (3×50 mL). The combined organic layers were washed with saturated NaCl solution (3×50 mL), dried over Na₂SO₄, filtered and concentrated in vacuo to give the title compound (6.60 g, 91% yield) as a yellow solid. ¹H NMR (400 MHz, CDCl₃) δ 10.23 (s, 1H), 8.18 (s, 1H), 7.22 (s, 1H), 4.87-4.70 (m, 2H), 4.11-4.09 (m, 1H), 4.00 (dd, J=1.6, 8.0 Hz, 1H), 3.49 (d, J=10.0 Hz, 1H), 2.08 (s, 2H), 1.65 (s, 1H).

### Step 2—[4-[5-Bromo-6-[(1S,4S)-2-oxa-5-azabicyclo[2.2.1]heptan-5-yl]indazol-2-yl]cyclohexyl] methanol

To a solution of 5-bromo-2-nitro-4-[(1S,4S)-2-oxa-5-azabicyclo[2.2.1]heptan-5-yl]benzaldehyde (2.00 g, 6.11 mmol) in IPA (50 mL) was added (4-aminocyclohexyl)methanol (948 mg, 7.34 mmol, Intermediate ATD). The mixture was heated at 80° C. for 4 hr under N₂. Then the mixture was cooled to 25° C. and tributylphosphane (3.71 g, 18.3 mmol) was added. The reaction mixture was heated to 80° C. for 16 hr. On completion, the reaction mixture was concentrated in vacuo to give a residue. The residue was purified by reverse phase flash (0.1% FA condition) to give the title compound (4.00 g, 81% yield) as a yellow solid. ¹H NMR (400 MHz, DMSO-d₆) δ 8.24 (s, 1H), 7.95 (s, 1H), 7.14 (s, 1H), 4.57 (s, 1H), 4.41-4.29 (m, 2H), 3.97 (d, J=7.6 Hz, 1H), 3.74 (dd, J=1.6, 7.6 Hz, 1H), 3.53 (dd, J=1.6, 9.6 Hz, 2H), 3.30-3.23 (m, 4H), 2.18-2.05 (m, 2H), 1.96-1.76 (m, 6H), 1.20-1.09 (m, 2H).

### N-[2-(4-formylcyclohexyl)-6-[(1S,4S)-2-oxa-5-azabicyclo[2.2.1]heptan-5-yl]indazol-5-yl]-6-(trifluoromethyl)pyridine-2-carboxamide (Intermediate BAS)

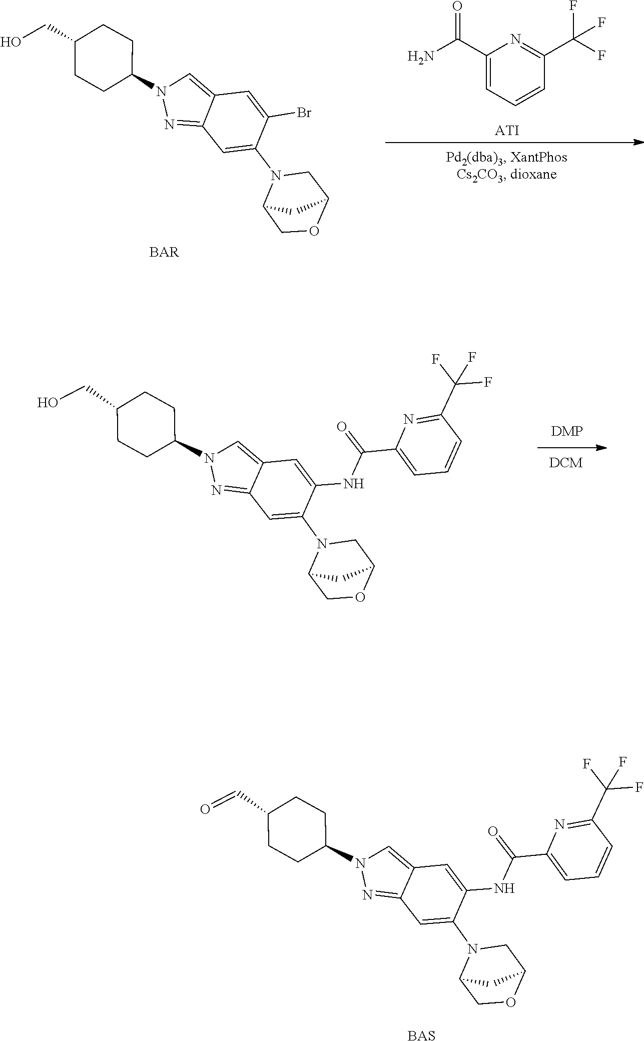

### Step 1—N-[2-[4-(hydroxymethyl)cyclohexyl]-6-[(1S,4S)-2-oxa-5-azabicyclo[2.2.1]heptan-5-yl]indazol-5-yl]-6-(trifluoromethyl)pyridine-2-carboxamide

To a solution of [4-[5-bromo-6-[(1S,4S)-2-oxa-5-azabicyclo[2.2.1]heptan-5-yl]indazol-2-yl] cyclohexyl]methanol (2.00 g, 4.92 mmol, Intermediate BAR) in dioxane (50 mL) was added Pd₂(dba)₃ (283 mg, 492 umol), Xantphos (570 mg, 984 umol), Cs₂CO₃ (3.21 g, 9.84 mmol) and 6-(trifluoromethyl)pyridine-2-carboxamide (1.40 g, 7.38 mmol, Intermediate ATI). The mixture was stirred at 110° C. for 16 hrs. On completion, the reaction mixture was filtered and the filtrate was concentrated in vacuo to give a residue. The residue was purified by reverse phase flash (0.1% FA condition) to give a residue. The residue was dissolved in solvent EtOAc, and the resulting suspension was stirred 30 min at 25° C., then the suspension was filtered and the filter cake was dried in vacuo to give the title compound (1.40 g, 55% yield). ¹H NMR (400 MHz, DMSO-d₆) δ 10.69 (s, 1H), 8.68 (s, 1H), 8.54-8.38 (m, 2H), 8.34 (s, 1H), 8.22 (d, J=7.6 Hz, 1H), 7.40 (s, 1H), 4.62 (s, 1H), 4.54-4.27 (m, 2H), 4.12-3.94 (m, 2H), 3.67 (dd, J=1.6, 7.6 Hz, 1H), 3.43 (d, J=10.0 Hz, 1H), 3.32-3.23 (m, 4H), 2.17-2.07 (m, 3H), 1.92-1.80 (m, 4H), 1.47 (m, 1H), 1.26-1.06 (m, 2H).

### Step 2—N-[2-(4-formylcyclohexyl)-6-[(1S,4S)-2-oxa-5-azabicyclo[2.2.1]heptan-5-yl]indazol-5-yl]-6-(trifluoromethyl)pyridine-2-carboxamide

To a mixture of N-[2-[4-(hydroxymethyl)cyclohexyl]-6-[(1S,4S)-2-oxa-5-azabicyclo[2.2.1]heptan-5-yl]indazol-5-yl]-6-(trifluoromethyl)pyridine-2-carboxamide (201 mg, 390 umol) in DCM (5.0 mL) was added DMP (198 mg, 467 umol) at 0° C. under N₂. The mixture was stirred at 0° C. for 30 min. On completion, the mixture was quenched with saturated Na₂S₂O₃ (10 mL) and washed with saturated NaHCO₃ (2×10 mL). The organic layer was dried over anhydrous Na₂SO₄, filtered and concentrated in vacuo to give the title compound (200 mg, 94% yield) as yellow solid. LC-MS (ESI⁺) m/z 514.2 (M+H)⁺.

### N-[2-(4-formylcyclohexyl)indazol-5-yl]-6-(trifluoromethyl)pyridine-2-carboxamide (Intermediate BRP)

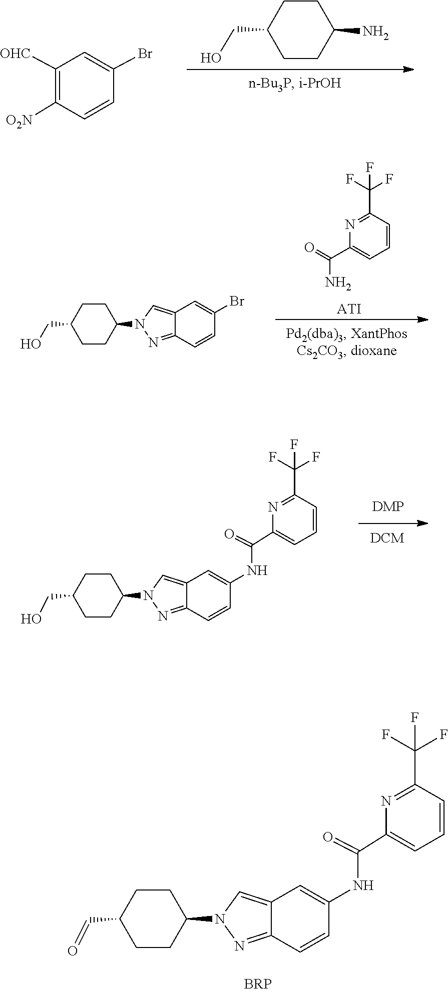

### Step 1—[4-(5-Bromoindazol-2-yl)cyclohexyl]methanol

To a solution of 5-bromo-2-nitro-benzaldehyde (2.00 g, 8.70 mmol, CAS #20357-20-4) in i-PrOH (30 mL) was added and (4-aminocyclohexyl)methanol (1.24 g, 9.56 mmol, CAS #1467-84-1). The mixture was stirred at 80° C. for 5 hours, then the tributylphosphane (5.28 g, 26.0 mmol, 6.44 mL) was added at 25° C. Next, the reaction mixture was stirred at 80° C. for 5 hours. On completion, the reaction mixture was concentrated in vacuo. The residue was purified by column chromatography then the residue was triturated with PE (2 mL) and filtered to give the title compound (1.00 g, 37% yield) as yellow solid. ¹H NMR (400 MHz, DMSO-d₆) δ 8.39 (d, J=0.8 Hz, 1H), 7.93 (dd, J=0.4, 2.0 Hz, 1H), 7.61-7.56 (m, 1H), 7.29 (dd, J=2.0, 9.2 Hz, 1H), 4.49 (t, J=5.2 Hz, 1H), 4.47-4.40 (m, 1H), 2.13-2.10 (m, 2H), 1.94-1.83 (m, 4H), 1.63-1.32 (m, 3H), 1.21-1.08 (m, 2H). LC-MS (ESI⁺) m/z 309.1 (M+H)⁺.

### Step 2—N-[2-[4-(hydroxymethyl)cyclohexyl]indazol-5-yl]-6-(trifluoromethyl)pyridine-2-carboxamide

A mixture of [4-(5-bromoindazol-2-yl)cyclohexyl]methanol (300 mg, 970 umol), 6-(trifluoromethyl) pyridine-2-carboxamide (276. mg, 1.46 mmol, Intermediate ATI), Cs₂CO₃ (632 mg, 1.94 mmol), Pd₂(dba)₃ (88.8 mg, 97.0 umol) and Xantphos (112 mg, 194 umol) in dioxane (8 mL) was purged with N₂ three times. Then the mixture was stirred at 100° C. for 6 hours under N₂ atmosphere. On completion, the filtrate was concentrated in vacuo. The residue was purified by column chromatography to give the title compound (328 mg, 80% yield) as yellow solid. ¹H NMR (400 MHz, DMSO-d₆) δ 10.35 (s, 1H), 8.42-8.38 (m, 2H), 8.38-8.32 (m, 1H), 8.29 (d, J=1.2 Hz, 1H), 8.17 (dd, J=1.2, 7.6 Hz, 1H), 7.63-7.59 (m, 1H), 7.57-7.52 (m, 1H), 4.53-4.37 (m, 2H), 2.16-2.14 (m, 2H), 1.94-1.86 (m, 4H), 1.56-1.47 (m, 1H), 1.24-1.08 (m, 4H). LC-MS (ESI⁺) m/z 419.5 (M+H)⁺.

### Step 3—N-[2-(4-formylcyclohexyl)indazol-5-yl]-6-(trifluoromethyl)pyridine-2-carboxamide

To a solution of N-[2-[4-(hydroxymethyl)cyclohexyl]indazol-5-yl]-6-(trifluoromethyl)pyridine-2-carboxamide (100 mg, 239 umol) in DCM (1 mL) was added DMP (121 mg, 286 umol) at 25° C. and the mixture was stirred for 10 minutes. On completion, the reaction mixture was quenched with Na₂S₂O₃ (2 mL) and NaHCO₃ (2 mL) at 25° C., and extracted with DCM (4×3 mL). The combined organic layers were dried over anhydrous Na₂SO₄, filtered and concentrated in vacuo to give the title compound (85.0 mg, 85% yield) as yellow solid. ¹H NMR (400 MHz, DMSO-d₆) δ 10.36 (s, 1H), 9.65 (s, 1H), 8.43-8.33 (m, 3H), 8.30 (d, J=1.2 Hz, 1H), 8.17 (dd, J=1.2, 7.6 Hz, 1H), 7.64-7.53 (m, 2H), 4.47 (m, 1H), 2.26-2.07 (m, 4H), 1.99-1.94 (m, 2H), 1.50-1.44 (m, 2H), 1.23 (s, 1H). LC-MS (ESI⁺) m/z 417.1 (M+H)⁺.

### N-[6-fluoro-2-(4-formylcyclohexyl)-1-methyl-indol-5-yl]-6-(trifluoromethyl)pyridine-2-carboxamide (Intermediate BRQ)

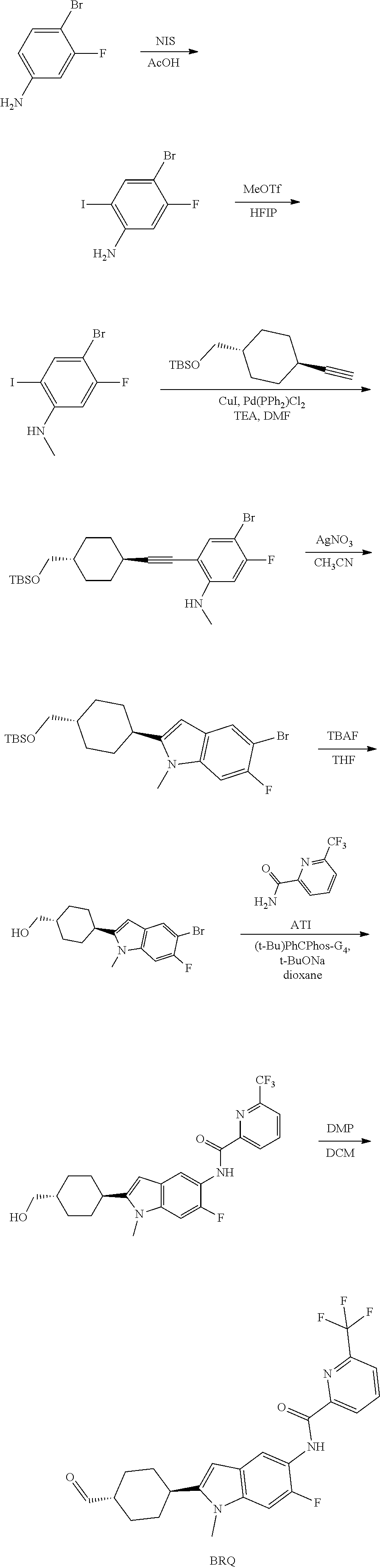

### Step 1—4-Bromo-5-fluoro-2-iodo-aniline

To a solution of 4-bromo-3-fluoro-aniline (2.00 g, 10.5 mmol, CAS #656-65-5) in HOAc (40 mL) was added NIS (2.84 g, 12.63 mmol) and the mixture was stirred at 25° C. for 2 hrs. On completion, the reaction mixture was concentrated in vacuo to give a residue. The residue was diluted with EA (50 mL) and quenched with saturated NaHCO₃ until the pH=7. The organic layer separated and dried over Na₂SO₄, filtered and concentrated in vacuo to give a residue. The residue was purified by column chromatography (SiO₂, PE/EA=40:1) to give the title compound (1.70 g, 51% yield) as brown solid. ¹H NMR (400 MHz, DMSO-d₆) δ 7.75 (d, J=7.6 Hz, 1H), 6.67 (d, J=11.2 Hz, 1H), 5.67 (s, 2H).

### Step 2—4-Bromo-5-fluoro-2-iodo-N-methyl-aniline

To a solution of 4-bromo-5-fluoro-2-iodo-aniline (1.60 g, 5.06 mmol) in HFIP (20 mL) was added methyl trifluoromethanesulfonate (831 mg, 5.06 mmol) dropwise at 0° C., then the mixture was stirred at 25° C. for 48 hrs. On completion, the reaction was quenched with NaHCO₃ (30 mL) and extracted with EA (30 mL×3). The combined organic layer was dried over Na₂SO₄, filtered and concentrated in vacuo to give a residue. The residue was purified by column chromatography (SiO₂, PE/EA=1:0) to give the title compound (1.00 g, 59% yield) was obtained as light yellow solid. ¹H NMR (400 MHz, DMSO-d₆) δ 7.80 (d, J=7.6 Hz, 1H), 6.48 (d, J=12.4 Hz, 1H), 5.46 (d, J=3.2 Hz, 1H), 2.73 (d, J=4.8 Hz, 3H).

### Step 3—4-Bromo-2-[2-[4-[[tert-butyl(dimethyl)silyl]oxymethyl]cyclohexyl]ethynyl]-5-fluoro-N-methyl-aniline

To a solution of 4-bromo-5-fluoro-2-iodo-N-methyl-aniline (0.80 g, 2.42 mmol) and tert-butyl-[(4-ethynylcyclohexyl)methoxy]-dimethyl-silane (734 mg, 2.91 mmol, Intermediate BRH) in DMF (10 mL) was added TEA (1.23 g, 12.1 mmol), CuI (46.1 mg, 242 umol) and Pd(PPh₃)₂Cl₂ (340 mg, 484 umol). The mixture was stirred at 25° C. for 3 hrs. On completion, the reaction mixture was diluted with H₂O (40 mL) and extracted EA (20 mL×3). The combined organic layer was dried over Na₂SO₄, filtered and concentrated in vacuo to give a residue. The residue was purified by reverse phase (FA condition) to give the title compound (0.8 g, 72% yield) as light yellow oil. ¹H NMR (400 MHz, CDCl3) δ 7.32 (d, J=7.6 Hz, 1H), 6.28 (d, J=11.2 Hz, 1H), 3.37 (d, J=6.4 Hz, 2H), 2.82 (s, 3H), 3.41-2.34 (m, 2H), 2.08-2.01 (m, 2H), 1.94 (dd, J=3.2, 13.2 Hz, 1H), 1.82-1.75 (m, 2H), 1.66-1.57 (m, 1H), 1.45-1.37 (m, 3H), 1.00-0.89 (m, 2H), 0.86 (s, 9H), 0.01 (s, 6H).

### Step 4—[4-(5-Bromo-6-fluoro-1-methyl-indol-2-yl)cyclohexyl]methoxy-tert-butyl-dimethyl-silane

To a solution of 4-bromo-2-[2-[4-[[tert-butyl(dimethyl)silyl]oxymethyl]cyclohexyl]ethynyl]-5-fluoro-N-methyl-aniline (700 mg, 1.54 mmol) in CH₃CN (7 mL) was added AgNO₃ (26.1 mg, 154 umol) and the mixture was stirred at 80° C. for 16 hrs. On completion, the mixture was filtered and concentrated in vacuo to give a residue, then the residue was diluted with EA (30 mL) and washed with saturated NaCl (20 mL). The organic lay was dried over Na₂SO₄, filtered and concentrated in vacuo to give the title compound (0.7 g, 85% yield) as light yellow solid. ¹H NMR (400 MHz, CDCl₃) δ 7.65 (d, J=6.8 Hz, 1H), 7.03 (d, J=9.2 Hz, 1H), 6.16 (s, 1H), 3.64 (s, 3H), 3.48 (d, J=6.4 Hz, 2H), 2.68-2.55 (m, 1H), 2.12-2.03 (m, 2H), 1.97-1.92 (m, 2H), 1.55-1.40 (m, 3H), 1.19-1.06 (m, 2H), 0.92 (s, 9H), 0.07 (s, 6H).

### Step 5—[4-(5-Bromo-6-fluoro-1-methyl-indol-2-yl)cyclohexyl]methanol

To a solution of [4-(5-bromo-6-fluoro-1-methyl-indol-2-yl)cyclohexyl]methoxy-tert-butyl-dimethyl-silane (500 mg, 1.10 mmol) in THF (10 mL) was added TBAF (1 M, 2.20 mmol, 2.20 mL) and the mixture was stirred at 25° C. for 1 hr. On completion, the reaction mixture was diluted with EA (50 mL) and washed with saturated NaCl (20 mL×2). The organic layer was dried over Na₂SO₄, filtered and concentrated in vacuo to give the title compound (0.36 g, 80% yield) as light yellow solid. ¹H NMR (400 MHz, CDCl₃) δ 7.65 (d, J=6.8 Hz, 1H), 7.04 (d, J=9.6 Hz, 1H), 6.17 (s, 1H), 3.64 (s, 3H), 3.55 (s, 2H), 2.71-2.58 (m, 1H), 2.10 (d, J=12.4 Hz, 2H), 1.98 (d, J=12.0 Hz, 2H), 1.68-1.60 (m, 1H), 1.55-1.44 (m, 2H), 1.36-1.28 (m, 1H), 1.26-1.10 (m, 2H).

### Step 6—N-[6-fluoro-2-[4-(hydroxymethyl)cyclohexyl]-1-methyl-indol-5-yl]-6-(trifluoromethyl) pyridine-2-carboxamide

To a solution of [4-(5-bromo-6-fluoro-1-methyl-indol-2-yl)cyclohexyl]methanol (200 mg, 587 umol) and 6-(trifluoromethyl)pyridine-2-carboxamide (122 mg, 646 umol, Intermediate ATI) in dioxane (4 mL) was added (t-Bu)PhCPhos Pd G₄ (20 mg) and t-BuONa (112 mg, 1.18 mmol). The mixture was stirred at 80° C. for 16 hrs. On completion, the reaction was diluted with water (10 mL) and extracted with EA (10 mL×3). The combined organic layers were dried over Na₂SO₄, filtered and concentrated in vacuo to give a residue. The residue was purified by column chromatography (SiO₂, petroleum ether/ethyl acetate=1/1) and then re-purified by prep-TLC (PE/EA=1/1) to give the title compound (15.0 mg, 5% yield) as light yellow solid. ¹H NMR (400 MHz, CDCl₃) δ 10.08 (s, 1H), 8.63-8.45 (m, 2H), 8.17-8.08 (m, 1H), 7.87 (d, J=8.0 Hz, 1H), 7.08 (d, J=11.6 Hz, 1H), 6.26 (s, 1H), 3.67 (s, 3H), 3.50-3.44 (m, 3H), 2.35-2.25 (m, 2H), 2.02-1.98 (m, 2H), 1.82-1.78 (m, 1H), 1.04-0.92 (m, 4H); LC-MS (ESI⁺) m/z 450.1 (M+H)⁺.

### Step 7—N-[6-fluoro-2-(4-formylcyclohexyl)-1-methyl-indol-5-yl]-6-(trifluoromethyl)pyridine-2-carboxamide

To a solution of N-[6-fluoro-2-[4-(hydroxymethyl)cyclohexyl]-1-methyl-indol-5-yl]-6-(trifluoro methyl)pyridine-2-carboxamide (13.0 mg, 28.9 umol) in DCM (1 mL) was added DMP (14.7 mg, 34.7 umol) and the mixture was stirred at 25° C. for 16 hrs. On completion, the reaction was diluted with DCM (10 mL) and quenched with saturated Na₂S₂O₃ (3 mL) and NaHCO₃ (10 mL). The organic layer was separated and dried over Na₂SO₄, filtered and concentrated in vacuo to give the title compound (12.0 mg, 90% yield) as light yellow solid. LC-MS (ESI⁺) m/z 448.3 (M+H)⁺.

### 1-Methyl-3-[3-methyl-2-oxo-4-(4-piperidyl)benzimidazol-1-yl]piperidine-2,6-dione (Intermediate BCD)

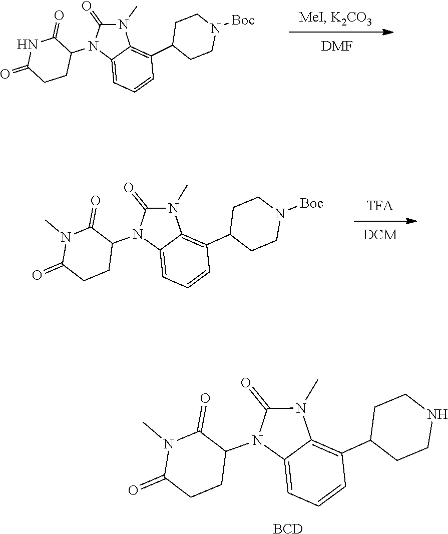

### Step 1—Tert-butyl 4-[3-methyl-1-(1-methyl-2,6-dioxo-3-piperidyl)-2-oxo-benzimidazol-4-yl]piperidine-1-carboxylate

To a solution of tert-butyl 4-[1-(2,6-dioxo-3-piperidyl)-3-methyl-2-oxo-benzimidazol-4-yl]piperidine-1-carboxylate (300 mg, 677 umol, synthesized via Steps 1-2 of Intermediate AZK) in DMF (4 mL) was added K₂CO₃ (93.7 mg, 677 umol). The reaction mixture was stirred at 0° C. for 15 min. Then MeI (192 mg, 1.36 mmol) was added and the reaction mixture was stirred at 25° C. for 4 hrs. On completion, the mixture was quenched with water (1 mL) and extracted with ethyl acetate (3×25 mL). The combined organic phase was dried over anhydrous sodium sulfate, filtered and concentrated to give a residue. The residue was triturated with NH₄Cl (20 mL) at 25° C. for 15 min to give the title compound (250 mg, 80% yield) as an off-white solid. ¹H NMR (400 MHz, DMSO-d₆) δ 7.22 (s, 2H), 6.99 (s, 1H), 4.22-3.96 (m, 2H), 3.63-3.59 (m, 3H), 3.48-3.38 (m, 1H), 3.03 (s, 3H), 2.99-2.91 (m, 2H), 2.89 (s, 1H), 2.83-2.64 (m, 3H), 2.06-1.94 (m, 1H), 1.82 (d, J=12.4 Hz, 2H), 1.66-1.50 (m, 2H), 1.43 (s, 9H), LC-MS (ESI⁺) m/z 401.3 (M+1)⁺.

### Step 2—1-Methyl-3-[3-methyl-2-oxo-4-(4-piperidyl)benzimidazol-1-yl]piperidine-2,6-dione

To a solution of tert-butyl 4-[3-methyl-1-(1-methyl-2,6-dioxo-3-piperidyl)-2-oxo-benzimidazol-4-yl]piperidine-1-carboxylate (250 mg, 547 umol) in DCM (3 mL) was added TFA (1 mL). The reaction mixture was stirred at 25° C. for 1 hr. On completion, the mixture was concentrated to give the title compound (100 mg, 38% yield, TFA) as brown oil. LC-MS (ESI⁺) m/z 357.1 (M+1)⁺.

### N-[2-[4-(hydroxymethyl)cyclohexyl]-6-(1-hydroxy-1-methyl-ethyl)indazol-5-yl]-6-methyl-pyridine-2-carboxamide (Intermediate AOX)

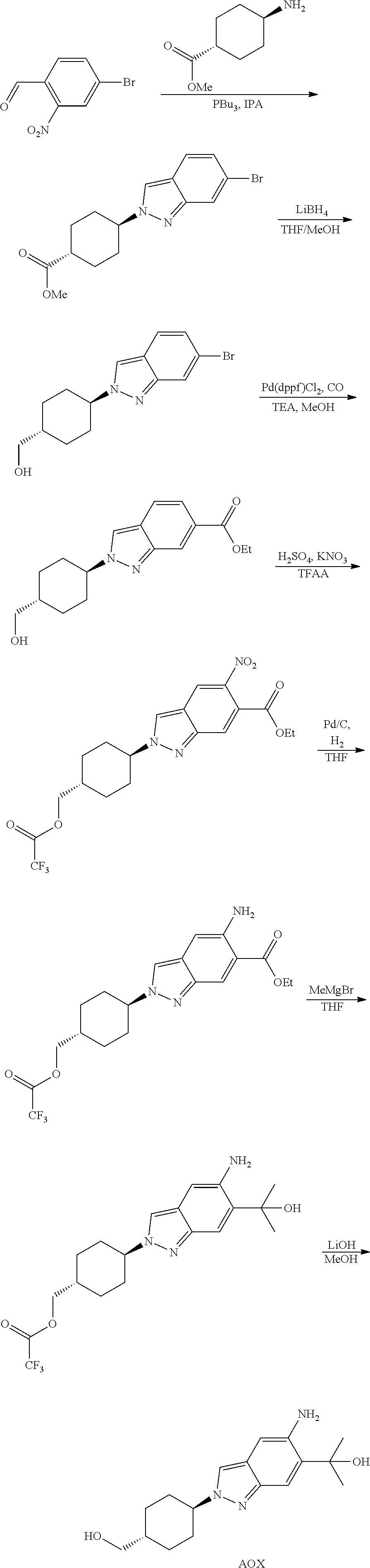

### Step 1—(1r, 4r)-Methyl 4-(6-bromo-2H-indazol-2-yl)cyclohexanecarboxylate

To a solution of 4-bromo-2-nitro-benzaldehyde (10.0 g, 43.5 mmol, CAS #62456-15-9) in IPA (100 mL) was added methyl 4-aminocyclohexanecarboxylate (6.83 g, 43.5 mmol, CAS #5551-12-2). The mixture was heated at 80° C. for 4 hrs under N₂. It was cooled to 25° C. Tributylphosphane (26.4 g, 130 mmol) was added and the reaction mixture was heated to 80° C. for 16 hrs. On completion, the reaction mixture was concentrated in vacuo to give a residue. The residue was purified by silica gel chromatography (PE:EA=10:1) to give the title compound (13.0 g, 87% yield) as a white solid. ¹H NMR (400 MHz, CDCl₃) δ 7.94 (d, J=0.8 Hz, 1H), 7.92-7.85 (m, 1H), 7.53 (d, J=8.8 Hz, 1H), 7.16 (dd, J=1.6, 8.8 Hz, 1H), 4.41 (tt, J=4.0, 12.0 Hz, 1H), 3.73 (s, 3H), 2.47 (tt, J=3.6, 12.4 Hz, 1H), 2.41-2.32 (m, 2H), 2.30-2.21 (m, 2H), 2.01 (dq, J=3.2, 12.8 Hz, 2H), 1.60-1.56 (m, 2H); LC-MS (ESI⁺) m/z 339.0 (M+H)⁺.

### Step 2—((1r, 4r)-4-(6-Bromo-2H-indazol-2-yl)cyclohexyl)methanol

To a solution of methyl 4-(6-bromoindazol-2-yl)cyclohexanecarboxylate (9.00 g, 26.7 mmol) in a mixed solvent of tertahydrofuran (90.0 mL) and methanol (11.0 mL) was added LiBH4 (1.74 g, 80.0 mmol) at 25° C. The reaction mixture was stirred at 60° C. for 3 hr. On completion, the reaction mixture was quenched with saturated NH₄Cl (100 mL), then removed to terthydrofuran and methanol. The residue was extracted with dichloromethane (3×100 mL). The combined organic layers were washed with brine (50 mL), dried over anhydrous sodium sulfate, filtered and the filtrate was concentrated in vacuo to give the title compound (7.80 g, 95% yield) as a white solid. ¹H NMR (400 MHz, CDCl₃) δ 7.95 (s, 1H), 7.92-7.89 (m, 1H), 7.54 (d, J=8.8 Hz, 1H), 7.16 (dd, J=1.6, 8.8 Hz, 1H), 4.75 (s, 1H), 4.39 (tt, J=3.6, 12.0 Hz, 1H), 3.58 (d, J=6.4 Hz, 2H), 2.47-2.26 (m, 2H), 2.14-1.91 (m, 4H), 1.68-1.63 (m, 1H), 1.27 (dq, J=3.2, 12.8 Hz, 2H), LC-MS (ESI⁺) m/z 311.2 (M+H)⁺.

### Step 3—Ethyl 2-((1r, 4r)-4-(hydroxymethyl)cyclohexyl)-2H-indazole-6-carboxylate

To a stirring solution of [4-(6-bromoindazol-2-yl)cyclohexyl]methanol (7.30 g, 23.6 mmol) in EtOH (70.0 mL) in a steel bomb under inert atmosphere were added Pd(dppf)Cl₂ (1.73 g, 2.36 mmol), TEA (11.9 g, 118 mmol, 16.4 mL) and the mixture was heated to 80° C. under CO gas atmosphere (50 psi) and stirred for 12 hrs. On completion, the reaction mixture was filtered and concentrated in vacuo to give a residue. The residue was purified by silica gel chromatography (PE:EA=2:1) to give the title compound (6.70 g, 93% yield) as a white solid. LC-MS (ESI⁺) m/z 301.1 (M+H)⁺.

### Step 4—Ethyl 5-nitro-2-((1r, 4r)-4-((2,2,2-trifluoroacetoxy)methyl)cyclohexyl)-2H-indazole-6-carboxylate

To a solution of ethyl 2-[4-(hydroxymethyl)cyclohexyl]indazole-6-carboxylate (6.5 g, 21.5 mmol) in TFAA (22.6 g, 107 mmol, 15 mL). The reaction mixture was stirred at 20° C. for 2 hrs. After, KNO₃ (4.35 g, 43.0 mmol) in H₂SO₄ (60 mL) was added dropwise at −5° C. The reaction mixture was stirred at 20° C. for 1 hr. On completion, the reaction mixture was poured into cold water (10 mL) and extracted with EA (3×100 mL). The combined organic layers were washed with brine (2×100 mL), saturated NaHCO₃ (5×200 mL) until the pH=7, dried over Na₂SO₄, filtered and concentrated in vacuo to give the title compound (9.30 g, 97% yield) as a yellow solid. ¹H NMR (400 MHz, CDCl₃) δ 8.41 (s, 1H), 8.22 (d, J=0.6 Hz, 1H), 8.04 (s, 1H), 4.56-4.45 (m, 1H), 4.41 (q, J=7.2 Hz, 2H), 4.29 (d, J=6.4 Hz, 2H), 2.45-2.35 (m, 2H), 2.11-2.03 (m, 4H), 2.00-1.91 (m, 1H), 1.44-1.31 (m, 5H). LC-MS (ESI⁺) m/z 444.1 (M+H)⁺.

### Step 5—Ethyl 5-amino-2-[4-[(2,2,2-trifluoroacetyl)oxymethyl]cyclohexyl]indazole-6-carboxylate

To a solution of ethyl 5-nitro-2-[4-[(2,2,2-trifluoroacetyl)oxymethyl]cyclohexyl]indazole-6-carboxylate (9.20 g, 20.7 mmol) in THF (150.0 mL) was added Pd/C (1.00 g, 10 wt %). The reaction mixture was stirred at 25° C. for 12 hrs under H₂ (15 Psi) atmosphere. On completion, the reaction mixture was filtered and the filtrate was concentrated in vacuo to give the title compound (8.30 g, 96% yield) as a yellow solid. LC-MS (ESI⁺) m/z 414.2 (M+H)⁺.

### Step 6—[4-[5-Amino-6-(1-hydroxy-1-methyl-ethyl)indazol-2-yl]cyclohexyl]methyl 2,2,2-trifluoroacetate

To a solution of ethyl 5-amino-2-[4-[(2,2,2-trifluoroacetyl)oxymethyl]cyclohexyl]indazole-6-carboxylate (8.30 g, 20.1 mmol) in THF (80.0 mL) was added MeMgBr (3 M, 53.5 mL) dropwise at 0° C. Then the reaction mixture was stirred at 20° C. for 12 hrs. On completion, the reaction mixture was quenched with water (400 mL), and extracted with EA (2×300 mL). The organic layer was washed with brine (100 mL), and concentrated in vacuo. The residue was purified by silica gel chromatography (SiO2, PE:EA=3:1) to give the title compound (5.00 g, 62% yield) as a yellow solid. LC-MS (ESI⁺) m/z 400.1 (M+H)⁺.

### Step 7—N-[2-[4-(hydroxymethyl)cyclohexyl]-6-(1-hydroxy-1-methyl-ethyl)indazol-5-yl]-6-methyl-pyridine-2-carboxamide

To a solution of [4-[5-amino-6-(1-hydroxy-1-methyl-ethyl)indazol-2-yl]cyclohexyl]methyl 2,2,2-trifluoroacetate (2.60 g, 6.51 mmol) in a mixed solvent of THF (15.0 mL) and H₂O (5.00 mL) was added LiOH·H₂O (1.37 g, 32.5 mmol). The reaction mixture was stirred at 60° C. for 20 hrs. On completion, the residue was poured into water (5 mL) and stirred for 5 minutes, then the aqueous phase was extracted with ethyl acetate (50 mL×2). The combined organic phase was washed with brine (2×50 mL), dried with anhydrous Na₂SO₄, filtered and concentrated in vacuo. The residue was purified by silica gel chromatography (column height: 250 mm, diameter: 100 mm, 100-200 mesh silica gel, DCM/MeOH=25/1, Rf=0.41) to afford the compound (840 mg, 35% yield) as yellow solid. ¹H NMR (400 MHz, DMSO-d₆) δ 7.88 (s, 1H), 7.31 (s, 1H), 6.63 (s, 1H), 5.35-5.15 (m, 3H), 4.48 (t, J=5.2 Hz, 1H), 4.32-4.22 (m, 1H), 3.27 (t, J=5.6 Hz, 2H), 2.12-2.03 (m, 2H), 1.92-1.80 (m, 4H), 1.59 (s, 6H), 1.50-1.40 (m, 1H), 1.18-1.05 (m, 2H). LC-MS (ESI⁺) m/z 304.1 (M+H)⁺.

### N-(2-((1r, 4r)-4-formylcyclohexyl)-6-(2-hydroxypropan-2-yl)-2H-indazol-5-yl)-2-methyloxazole-4-carboxamide (Intermediate BGF)

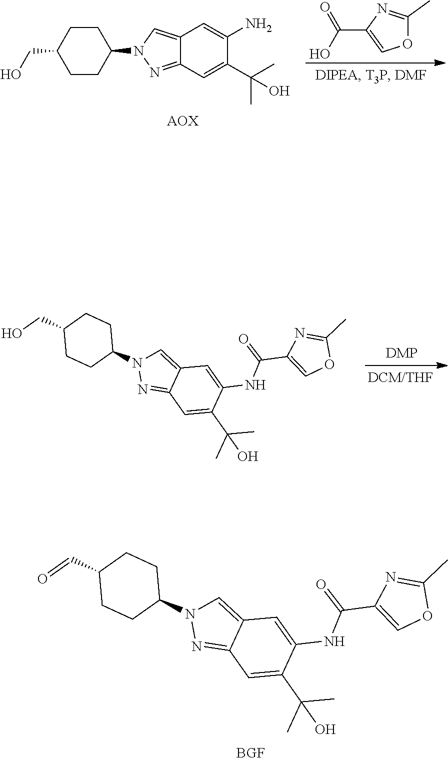

### Step 1—N-(2-((1r, 4r)-4-(hydroxymethyl)cyclohexyl)-6-(2-hydroxypropan-2-yl)-2H-indazol-5-yl)-2-methyloxazole-4-carboxamide

To a solution of 2-[5-amino-2-[4-(hydroxymethyl)cyclohexyl]indazol-6-yl]propan-2-ol (200 mg, 659 umol, Intermediate AOX), 2-methyloxazole-4-carboxylic acid (CAS #23062-17-1, 83.7 mg, 659 umol) in DMF (2 mL) was added DIPEA (1.28 g, 9.89 mmol) and T₃P (838 mg, 1.32 mmol, 50% solution) and the mixture was stirred at 20° C. for 12 hours. On completion, the reaction mixture was quenched with H₂O (0.5 mL). The mixture was purified by reverse phase flash (0.1% FA) to give the title compound (200 mg, 63% yield) as a yellow solid. LC-MS (ESI⁺) m/z 413.2 (M+H)⁺.

### Step 2—N-(2-((1r, 4r)-4-formylcyclohexyl)-6-(2-hydroxypropan-2-yl)-2H-indazol-5-yl)-2-methyloxazole-4-carboxamide

To a solution of N-[2-[4-(hydroxymethyl) cyclohexyl]-6-(1-hydroxy-1-methyl-ethyl)indazol-5-yl]-2-methyl-oxazole-4-carboxamide (180 mg, 436 umol) in DCM (9 mL) and THF (9 mL) was added DMP (222 mg, 523 umol) at 0° C. The mixture was stirred at 0-25° C. for 1 hour. On completion, the reaction mixture was quenched with H₂O (10 mL) at 25° C. and extracted with EA (3×10 mL). The combined organic layers were washed with brine (2×10 mL), dried over Na₂SO₄ and filtered. The filtrate was concentrated in vacuo to give the title compound (175 mg, 95% yield) as a brown solid. LC-MS (ESI⁺) m/z 411.4 (M+H)⁺.

### 1-Methyl-3-[3-methyl-4-[4-(methylamino)-1-piperidyl]-2-oxo-benzimidazol-1-yl]piperidine-2,6-dione (Intermediate BFL)

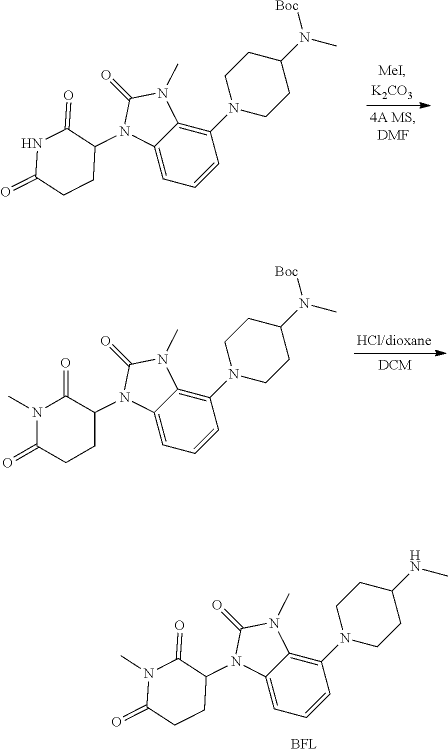

### Step 1—Tert-butyl N-methyl-N-[1-[3-methyl-1-(1-methyl-2,6-dioxo-3-piperidyl)-2-oxo-benzimidazol-4-yl]-4-piperidyl]carbamate

To a solution of tert-butyl N-[1-[1-(2, 6-dioxo-3-piperidyl)-3-methyl-2-oxo-benzimidazol-4-yl]-4-piperidyl]-N-methyl-carbamate (353 mg, 742 umol, synthesized via Step 1 of Intermediate AQK) in DMF (5 mL) was added 4 Å molecular sieves (30.0 mg), K₂CO₃ (113 mg, 816 umol) at 25° C. The reaction mixture was stirred for 10 minutes. Then MeI (158 mg, 1.11 mmol, 69.3 uL) was added at 0° C. and the mixture was stirred at 25° C. for 16 hours. On completion, the reaction mixture was filtered and the filtrate was adjusted to pH 4-5 with FA. The residue was poured into ice-water (10 mL) and stirred for 2 min. The aqueous phase was then extracted with ethyl acetate (2×10 mL). The combined organic layer was washed with brine (2×20 mL), dried with anhydrous Na₂SO₄, filtered and concentrated in vacuo. The residue was purified by silica gel chromatography (ethyl acetate) to give the title compound (240 mg, 66% yield) as yellow solid. LC-MS (ESI⁺) m/z 486.2 (M+H)⁺.

### Step 2—1-Methyl-3-[3-methyl-4-[4-(methylamino)-1-piperidyl]-2-oxo-benzimidazol-1-yl]piperidine-2,6-dione

To a solution of tert-butyl N-methyl-N-[1-[3-methyl-1-(1-methyl-2,6-dioxo-3-piperidyl)-2-oxo-benzimidazol-4-yl]-4-piperidyl]carbamate (100 mg, 206 umol) in DCM (3 mL) was added HCl/dioxane (4 M, 3 mL) and the mixture was stirred at 25° C. for 2 hours. On completion, the reaction mixture was concentrated in vacuo to give the title compound (86.0 mg, 99% yield, HCl salt) as brown solid. LC-MS (ESI⁺) m/z 386.1 (M+H)⁺.

### N-[2-(4-formylcyclohexyl)pyrazolo[3,4-c]pyridin-5-yl]-6-(trifluoromethyl)pyridine-2-carboxamide (Intermediate BRR)

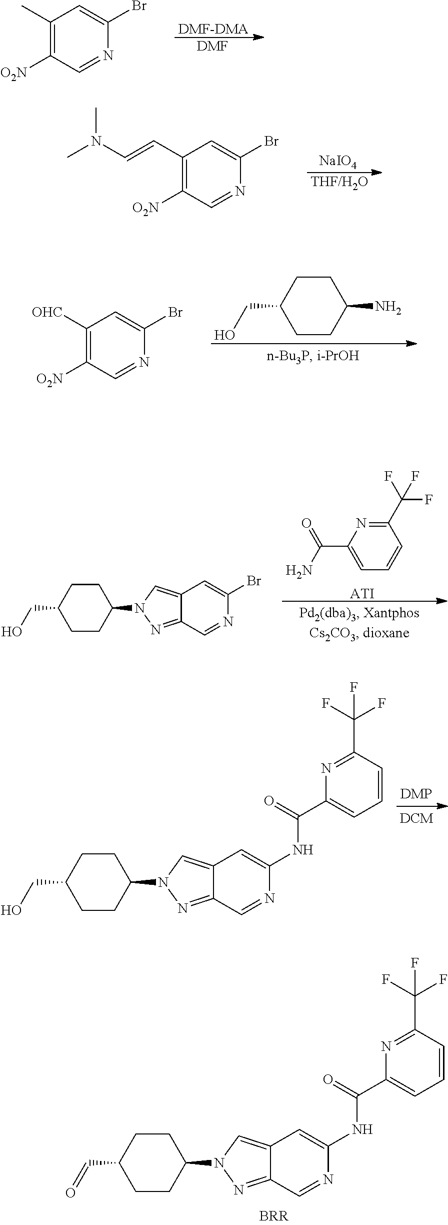

### Step 1—(E)-2-(2-Bromo-5-nitro-4-pyridyl)-N,N-dimethyl-ethenamine

To a solution of 2-bromo-4-methyl-5-nitro-pyridine (10.0 g, 46.0 mmol, CAS #23056-47-5) in DMF (160 mL) was added DMF-DMA (10.9 g, 92.1 mmol) and the mixture was stirred at 60° C. for 2 hours. The reaction was then diluted with water (340 mL) and extracted with EA (60 mL×3). The combined organic layer was washed with an aqueous solution of NaCl (50 mL). The organic layer was dried over Na₂SO₄, filtered and concentrated in vacuo to give a residue. The residue was triturated with EA/PE (10/1, 100 mL), filtered to give the title compound (7.70 g, 80% yield) as brown solid. ¹H NMR (400 MHz, CDCl₃) δ 8.72 (s, 1H), 7.41 (s, 1H), 7.32 (d, J=13.2 Hz, 1H), 5.91 (d, J=13.2 Hz, 1H), 3.05 (s, 6H).

### Step 2—2-Bromo-5-nitro-pyridine-4-carbaldehyde

To a solution of (E)-2-(2-bromo-5-nitro-4-pyridyl)-N,N-dimethyl-ethenamine (6.70 g, 24.6 mmol) in THF (134 mL) and H₂O (134 mL) was added NaIO₄ (15.8 g, 73.8 mmol) at 20° C. for 16 hours. On completion, an aqueous solution of Na₂S₂O₃ (50 mL) was added into the reaction mixture. Then the reaction mixture was stirred at 25° C. for 10 mins. After filtration via filter paper, the filtrates were diluted with water (300 mL) and extracted with ethyl acetate (100 mL×3). The combined organic layers were washed with an aqueous solution of NaCl (50 mL). The organic layer was dried over Na₂SO₄, filtered and concentrated in vacuo to give a residue. The residue was purified by silica gel chromatography (PE/EA=100/1, 50/1, 30/1, 0/1) to give the title compound (3.00 g, 52% yield) as yellow oil. ¹H NMR (400 MHz, CDCl₃) δ 10.51 (s, 1H), 9.21 (s, 1H), 7.92 (s, 1H).

### Step 3—[4-(5-Bromopyrazolo[3,4-c]pyridin-2-yl)cyclohexyl]methanol

To a solution of 2-bromo-5-nitro-pyridine-4-carbaldehyde (3.00 g, 12.99 mmol) and (4-aminocyclohexyl) methanol (1.85 g, 14.2 mmol) in i-PrOH (80 mL) was added tributylphosphane (7.88 g, 38.9 mmol, CAS #1467-84-1). The mixture was stirred at 80° C. for 16 hours. On completion, the reaction mixture was diluted with water (200 mL) and extracted with EA (50 mL×2). The combined organic layers were dried over Na₂SO₄, filtered and concentrated in vacuo to give a residue. The residue was purified by silica gel chromatography (PE/EA=5/1, 1/1, 0/1), then the residue was triturated with PE (2 mL) for 30 mins. The title compound (0.870 g, 21% yield) was obtained as yellow solid. ¹H NMR (400 MHz, CDCl₃) δ 9.04 (s, 1H), 7.97 (s, 1H), 7.74 (d, J=1.2 Hz, 1H), 4.50-4.42 (m, 1H), 3.58 (d, J=6.0 Hz, 2H), 2.37-2.33 (m, 2H), 2.09-2.05 (m, 2H), 2.05-1.95 (m, 2H), 1.73-1.53 (m, 2H), 1.33-1.23 (m, 2H); LC-MS (ESI⁺) m/z 309.9 (M+H)⁺.

### Step 4—N-[2-[4-(hydroxymethyl)cyclohexyl]pyrazolo[3,4-c]pyridin-5-yl]-6-(trifluoromethyl)pyridine-2-carboxamide

To a solution of [4-(5-bromopyrazolo[3,4-c]pyridin-2-yl)cyclohexyl]methanol (500 mg, 1.61 mmol) and 6-(trifluoromethyl)pyridine-2-carboxamide (306 mg, 1.61 mmol, Intermediate ATI) in dioxane (20 mL) was added Pd₂(dba)₃ (147 mg, 161 umol), Xantphos (186 mg, 322 umol) and Cs₂CO₃ (1.58 g, 4.84 mmol) under N₂. The mixture was then stirred at 80° C. for 16 hours. On completion, the reaction mixture was filtered and the filtrate was diluted with water (50 mL) and extracted with EA (20 ml×2). Then the combined organic layer was washed with an aqueous of NaCl (10 mL). The organic layer was dried over Na₂SO₄, filtered and concentrated in vacuo to give the residue. The residue was purified by silica gel chromatography (PE/EA=10/1, 5/1, 1/1, 0/1) to give the title compound (180 mg, 26.63% yield) as white solid. ¹H NMR (400 MHz, CDCl₃) δ 10.45 (s, 1H), 9.11 (s, 1H), 8.66-8.51 (m, 2H), 8.15-7.88 (m, 3H), 4.52-4.45 (m, 1H), 3.59 (d, J=6.4 Hz, 2H), 2.39-2.36 (m, 2H), 2.10-1.98 (m, 5H), 1.81-1.71 (m, 1H), 1.44-1.42 (m, 1H), 0.96-0.85 (m, 1H); LC-MS (ESI⁺) m/z 420.3 (M+H)⁺.

### Step 5—N-[2-(4-formylcyclohexyl)pyrazolo[3,4-c]pyridin-5-yl]-6-(trifluoromethyl)pyridine-2-carboxamide

To a solution of N-[2-[4-(hydroxymethyl)cyclohexyl]pyrazolo[3,4-c]pyridin-5-yl]-6-(trifluoromethyl) pyridine-2-carboxamide (80.0 mg, 190 umol) in DCM (4 mL) was added DMP (121 mg, 286 umol, 88.58 uL). The mixture was stirred at 0° C. for 6 hours. On completion, the reaction mixture was filtered and the filtrate was washed with an aqueous of NaHCO₃ (5 mL) and Na₂S₂O₃ (5 mL) and extracted with DCM (10 mL). The combined organic layer was dried over Na₂SO₄, filtered and concentrated in vacuo to give a residue. The residue was purified by reverse phase (0.10% FA) to give the title compound (70.0 mg, 87% yield) as white solid. ¹H NMR (400 MHz, CDCl₃) δ 10.40 (s, 1H), 9.74 (s, 1H), 9.10 (s, 1H), 8.65-8.51 (m, 2H), 8.16-7.88 (m, 3H), 4.52-4.45 (m, 1H), 2.47-2.29 (m, 5H), 2.17-2.05 (m, 2H), 1.81-1.74 (m, 1H), 1.43 (s, 1H); LC-MS (ESI⁺) m/z 418.2 (M+H)⁺.

### 3-(3,6-Dimethyl-4-(4-(methylamino)piperidin-1-yl)-2-oxo-2,3-dihydro-1H-benzo[d]imidazol-1-yl)piperidine-2,6-dione (Intermediate BRS)

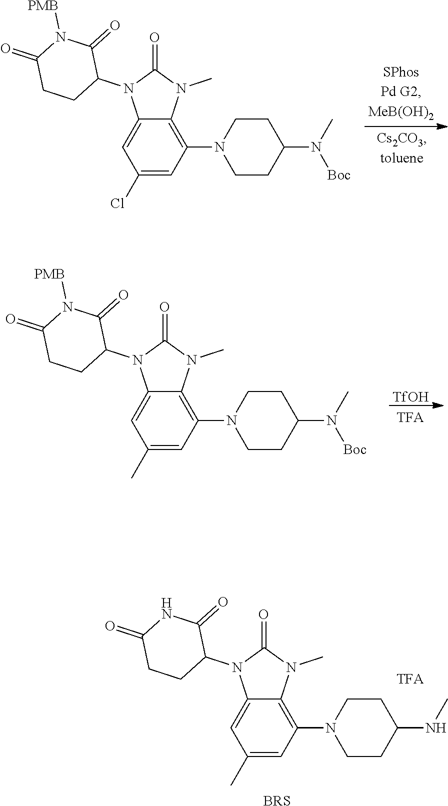

### Step 1—Tert-butyl (1-(1-(1-(4-methoxybenzyl)-2,6-dioxopiperidin-3-yl)-3,6-dimethyl-2-oxo-2,3-dihydro-1H-benzo[d]imidazol-4-yl)piperidin-4-yl)(methyl)carbamate

A mixture of tert-butyl N-[1-[6-chloro-1-[1-[(4-methoxyphenyl)methyl]-2,6-dioxo-3-piperidyl]-3-methyl-2-oxo-benzimidazol-4-yl]-4-piperidyl]-N-methyl-carbamate (100 mg, 159 umol, synthesized via Steps 1-4 of Intermediate BQQ), MeB(OH)₂ (191 mg, 3.19 mmol), Cs₂CO₃ (156 mg, 479 umol) and [2-(2-aminophenyl)phenyl]-chloro-palladium; dicyclohexyl-[2-(2,6-diisopropoxyphenyl)phenyl]phosphane (12.4 mg, 15.9 umol) in the toluene (2 mL) was stirred at 110° C. for 4 hrs under N₂. On completion, the reaction mixture was concentrated in vacuo and the residue was purified by prep-TLC (PE:EA=2:1) to give the title compound (90.0 mg, 93% yield) as a white solid. ¹H NMR (400 MHz, CDCl₃) δ 7.39 (d, J=8.6 Hz, 2H), 6.83 (d, J=8.7 Hz, 2H), 6.68 (s, 1H), 6.07 (s, 1H), 5.21 (dd, J=5.4, 13.6 Hz, 1H), 4.98 (s, 2H), 3.79 (s, 3H), 3.75 (s, 3H), 3.27-3.13 (m, 2H), 3.05-2.97 (m, 1H), 2.90-2.77 (m, 6H), 2.66-2.53 (m, 1H), 2.22 (s, 3H), 2.18-2.09 (m, 1H), 1.99-1.85 (m, 2H), 1.81-1.73 (m, 2H), 1.50 (s, 9H).

### Step 2—3-(3,6-Dimethyl-4-(4-(methylamino)piperidin-1-yl)-2-oxo-2,3-dihydro-1H-benzo[d]imidazol-1-yl)piperidine-2,6-dione

A mixture of tert-butyl N-[1-[1-[1-[(4-methoxyphenyl)methyl]-2,6-dioxo-3-piperidyl]-3,6-dimethyl-2-oxo-benzimidazol-4-yl]-4-piperidyl]-N-methyl-carbamate (90.0 mg, 148 umol) in the TFA (1 mL) and TfOH (0.2 mL) was stirred at 70° C. for 12 hrs. On completion, the reaction mixture was concentrated with a stream of N₂ and the residue was basified with TEA to pH=5˜6. The residue was then purified by reversed phase (FA, 0.1%) to give the title compound (55.0 mg, 96% yield) as a white solid. LC-MS (ESI⁺) m/z 386.1 (M+H)⁺.

### [4-(5-Bromo-6-fluoro-indazol-2-yl)cyclohexyl]methanol (Intermediate BRT)

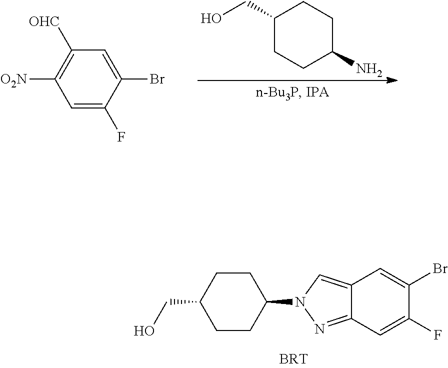 [SMILES: O=Cc1cc(Br)c(F)cc1[N+](=O)[O-] ; N[C@H]1CC[C@H](CO)CC1 ; OC[C@H]1CC[C@H](n2cc3cc(Br)c(F)cc3n2)CC1]

To a solution of 5-bromo-4-fluoro-2-nitro-benzaldehyde (2.00 g, 8.06 mmol, CAS #213382-45-7) in IPA (30 mL) was added (4-aminocyclohexyl)methanol (1.25 g, 9.68 mmol, CAS #1467-84-1), then the mixture was heated at 80° C. for 16 hours. The mixture was cooled to 25° C. then n-Bu₃P (4.89 g, 24.19 mmol) was added into the mixture, and the reaction was stirred at 80° C. for 16 hours. On completion, the reaction mixture was concentrated in vacuo to give the residue, then the residue was purified by flash silica gel chromatography to give the title compound (1.70 g, 64.43% yield) as yellow solid. ¹H NMR (400 MHz, DMSO-d₆) δ 7.84 (d, J=0.8 Hz, 1H), 7.80 (d, J=6.4 Hz, 1H), 7.32 (d, J=9.6 Hz, 1H), 4.29 (tt, J=4.0, 12.0 Hz, 1H), 3.48 (d, J=6.4 Hz, 2H), 2.31-2.20 (m, 2H), 2.00-1.82 (m, 4H), 1.61-1.55 (m, 2H), 1.26-1.12 (m, 2H).

### Pyrazolo[1,5-a]pyrimidine-3-carboxamide (Intermediate BRU)

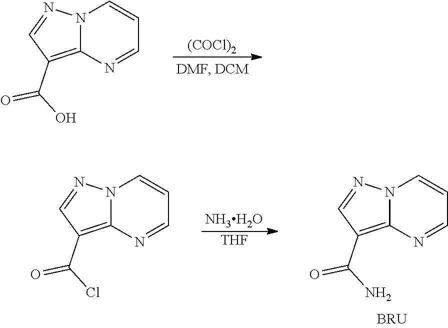

### Step 1—Pyrazolo[1,5-a]pyrimidine-3-carbonyl chloride

To a solution of pyrazolo[1,5-a]pyrimidine-3-carboxylic acid (2.00 g, 12.2 mmol, CAS #25940-35-6) in mixed solvent of DMF (1 mL) and DCM (100 mL) was added (COCl)₂ (6.99 g, 55.1 mmol, 4.83 mL) dropwise at 0° C. Then the mixture was stirred at 35° C. for 20 hours. On completion, the mixture was concentrated in vacuo to give the title compound (2.10 g, 94.33% yield).

### Step 2—Pyrazolo[1,5-a]pyrimidine-3-carboxamide

To a solution of NH₃·H₂O (35.6 mL, 231 mmol, 25% solution), and then pyrazolo[1,5-a]pyrimidine-3-carbonyl chloride (2.10 g, 11.5 mmol) in THF (20 mL) was added dropwise at 0° C. The resulting mixture was stirred at 0° C. for 3 hours. On completion, the mixture was concentrated in vacuo to remove solvent and filtered to give the title compound (1.80 g, 95.70% yield) as yellow solid. LC-MS (ESI⁺) m/z 146.1 (M−NH₂)⁺.

### N-[6-fluoro-2-(4-formylcyclohexyl)indazol-5-yl]pyrazolo[1,5-a]pyrimidine-3-carboxamide (Intermediate BRV)

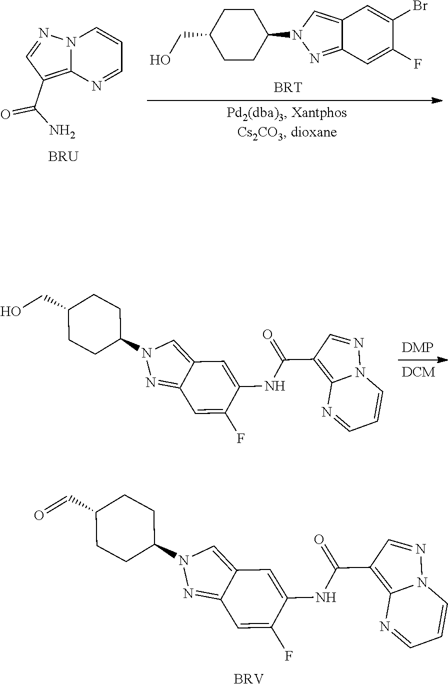 [SMILES: NC(=O)c1cnn2cccnc12 ; OC[C@H]1CC[C@H](n2cc3cc(Br)c(F)cc3n2)CC1 ; O=C(Nc1cc2cn([C@H]3CC[C@H](CO)CC3)nc2cc1F)c1cnn2cccnc12 ; O=C[C@H]1CC[C@H](n2cc3cc(NC(=O)c4cnn5cccnc45)c(F)cc3n2)CC1]

### Step 1—N-[6-fluoro-2-[4-(hydroxymethyl)cyclohexyl]indazol-5-yl]pyrazolo[1,5-a]pyrimidine-3-carboxamide

A mixture of pyrazolo[1,5-a]pyrimidine-3-carboxamide (371 mg, 2.29 mmol, Intermediate BRU), [4-(5-bromo-6-fluoro-indazol-2-yl)cyclohexyl]methanol (500 mg, 1.53 mmol, Intermediate BRT), Pd₂(dba)₃ (139 mg, 152 umol), Xantphos (176 mg, 305 umol) and Cs₂CO₃ (995 mg, 3.06 mmol) in dioxane (5 mL) was purged with N₂ three times. Then the mixture was stirred at 100° C. for 8 hours under N₂ atmosphere. On completion, the reaction mixture was filtered and concentrated in vacuo to give the residue, then the residue was diluted with water (50 mL) and extracted with EA (5×30 mL). The combined organic layer was dried over Na₂SO₄, filtered and concentrated in vacuo to give a residue. The residue was purified by reverse-phase to give the title compound (40 mg, 4.74% yield, 74% purity) as yellow solid. ¹H NMR (400 MHz, DMSO-d₆) δ 10.18 (d, J=3.2 Hz, 1H), 9.40 (dd, J=1.6, 7.2 Hz, 1H), 8.93 (dd, J=1.6, 4.4 Hz, 1H), 8.74 (s, 1H), 8.65 (d, J=8.0 Hz, 1H), 8.48-8.43 (m, 1H), 7.55 (s, 1H), 7.37-7.34 (m, 1H), 4.54-4.34 (m, 3H), 2.15-2.12 (m, 2H), 1.90-1.88 (m, 3H), 1.49-1.44 (m, 1H), 1.23 (s, 2H), 1.17-1.13 (m, 2H). LC-MS (ESI⁺) m/z 409.1 (M+H)⁺.

### Step 2—N-[6-fluoro-2-(4-formylcyclohexyl)indazol-5-yl]pyrazolo[1,5-a]pyrimidine-3-carboxamide

To a solution of N-[6-fluoro-2-[4-(hydroxymethyl)cyclohexyl]indazol-5-yl]pyrazolo[1,5-a]pyrimidine-3-carboxamide (35.0 mg, 85.7 umol) in DCM (1 mL) was added DMP (54.5 mg, 128 umol), then the mixture was stirred at 25° C. for 30 min. On completion, the mixture was quenched with sat. Na₂S₂O₃ (3 mL) and sat.NaHCO₃ (3 mL) under stirring. The mixture was extracted with DCM (5×5 mL), dried over Na₂SO₄, filtered and concentrated in vacuo to give the title compound (20.0 mg, 57.43%) as yellow solid. LC-MS (ESI⁺) m/z 407.1 (M+H)⁺.

### Ethyl 4-(p-tolylsulfonyloxy)cyclohexanecarboxylate (Intermediate AGK)

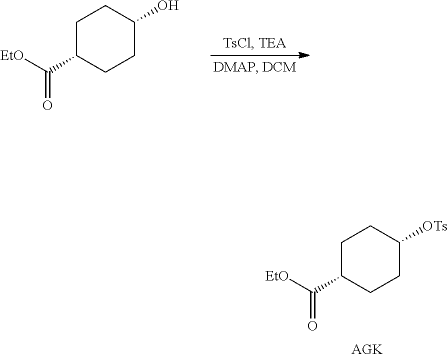 [SMILES: CCOC(=O)[C@H]1CC[C@@H](O)CC1 ; CCOC(=O)[C@H]1CC[C@@H](OS(=O)(=O)c2ccc(C)cc2)CC1]

To a solution of ethyl 4-hydroxycyclohexanecarboxylate (10.0 g, 58.06 mmol, CAS #75877-66-6), DMAP (710 mg, 5.81 mmol) and TEA (17.6 g, 174 mmol) in DCM (150 mL) was added p-TsCl (22.1 g, 116 mmol) at 15° C. The mixture was stirred at 15° C. for 16 hours. On completion, the reaction was quenched with water (20 mL) and the mixture was partitioned. The organic layer was concentrated in vacuo. The residue was purified by column chromatography on silica gel to give the title compound (16.0 g, 84% yield) as white solid. ¹H NMR (400 MHz, CDCl₃) δ 7.79 (d, J=8.2 Hz, 2H), 7.33 (d, J=7.9 Hz, 2H), 4.79-4.64 (m, 1H), 4.10 (q, J=7.2 Hz, 2H), 2.45 (s, 3H), 2.35-2.27 (m, 1H), 1.93-1.82 (m, 4H), 1.76-1.66 (m, 2H), 1.60-1.50 (m, 2H), 1.24 (t, J=7.2 Hz, 3H).

### N-[6-(1-hydroxy-1-methyl-ethyl)-2H-indazol-5-yl]-6-(trifluoromethyl)pyridine-2-carboxamide (Intermediate TJ)

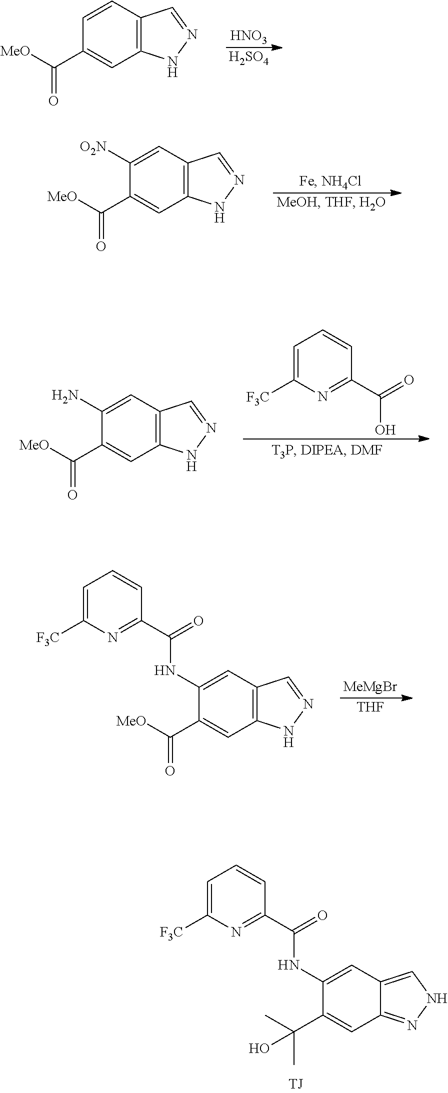

### Step 1—Methyl 5-nitro-1H-indazole-6-carboxylate

To a solution of methyl 1H-indazole-6-carboxylate (10.0 g, 56.7 mmol) in H₂SO₄ (100 mL) was added a solution of HNO₃ (12.1 g, 125 mmol, 65% solution) in H₂SO₄ (20 mL) at −10-0° C. during 30 minutes. The mixture was stirred at −10-0° C. for 1 hour. On completion, the mixture was poured into ice/water (1.0 L) slowly. The mixture was filtered and the filter cake was washed with water (2×200 mL). Then the cake was collected and dried in vacuo to give the title compound (11.9 g, 94% yield) as yellow solid. ¹H NMR (400 MHz, DMSO-d₆) δ 8.69 (s, 1H), 8.44 (s, 1H), 7.97 (s, 1H), 3.86 (s, 3H).

### Step 2—Methyl 5-amino-1H-indazole-6-carboxylate

To a solution of methyl 5-nitro-1H-indazole-6-carboxylate (10.9 g, 49.2 mmol) in MeOH (100 mL) and THF (60 mL) was added a solution of NH₄Cl(26.3 g, 492 mmol) in H₂O (100 mL) at 25° C. Then Fe (13.7 g, 245 mmol) was added to the mixture in portions at 70° C., and the mixture was stirred at 70° C. for 1 hour. On completion, the mixture was filtered and the filter cake was washed with EA (200 mL). The filtrate was concentrated in vacuo. The residue was washed with water (100 mL), and extracted with EA (3×100 mL). The combined organic layer was washed with brine (100 mL), dried over Na₂SO₄, filtered and concentrated in vacuo to the title compound (7.30 g, 77% yield) as yellow solid. ¹H NMR (400 MHz, DMSO-d₆) δ 12.82 (s, 1H), 7.99 (s, 1H), 7.85 (s, 1H), 6.99 (s, 1H), 6.00 (s, 2H), 3.85 (s, 3H).

### Step 3—Methyl 5-[[6-(trifluoromethyl)pyridine-2-carbonyl]amino]-1H-indazole-6-carboxylate

To a solution of methyl 5-amino-1H-indazole-6-carboxylate (7.20 g, 37.6 mmol), 6-(trifluoromethyl)pyridine-2-carboxylic acid (6.48 g, 33.9 mmol, CAS #131747-42-7) and DIPEA (7.35 g, 56.8 mmol) in THF (70 mL) was added T₃P (47.9 g, 44.8 mL, 50 wt %) slowly at 0° C. Then the mixture was stirred at 0-5° C. for 2 hours. On completion, the reaction was quenched with cold water (0.1 mL). The mixture was diluted with water (280 mL), and stirred at 25° C. for 0.5 hour. The mixture was filtered and the filter cake was washed with water (30 mL). The filter cake was collected and dried in vacuo to give the title compound (12.3 g, 99% yield) as yellow solid. ¹H NMR (400 MHz, DMSO-d₆) δ 12.58 (s, 1H), 9.15 (s, 1H), 8.47 (d, J=7.6 Hz, 1H), 8.39 (t, J=7.6 Hz, 1H), 8.30 (s, 1H), 8.25 (s, 1H), 8.20 (d, J=8.0 Hz, 1H), 3.97 (s, 3H).

### Step 4—N-[6-(1-hydroxy-1-methyl-ethyl)-2H-indazol-5-yl]-6-(trifluoromethyl)pyridine-2-carboxamide

To a solution of methyl 5-[[6-(trifluoromethyl)pyridine-2-carbonyl]amino]-1H-indazole-6-carboxylate (4.00 g, 10.9 mmol) in THF (40 mL) was added MeMgBr-Et₂O solution (3.0 M, 29.3 mL) slowly at 0° C. The mixture was stirred at 0-25° C. for 16 hours. On completion, the reaction was quenched with sat.NH₄Cl(40 mL) slowly at 0-10° C. The mixture was extracted with EA (3×40 mL). The combined organic layer was concentrated in vacuo. The residue was purified by reverse phase chromatography (FA condition) to give the title compound (1.50 g, 37% yield) as light yellow solid. ¹H NMR (400 MHz, CDCl₃) δ 12.23 (s, 1H), 8.96 (s, 1H), 8.52 (d, J=7.6 Hz, 1H), 8.12 (t, J=7.6 Hz, 1H), 8.07 (s, 1H), 7.85 (d, J=7.6 Hz, 1H), 7.50 (s, 1H), 1.80 (s, 6H).

### N-[2-(4-formylcyclohexyl)-6-(1-hydroxy-1-methyl-ethyl)indazol-5-yl]-6-(trifluoromethyl) pyridine-2-carboxamide (Intermediate AGL)

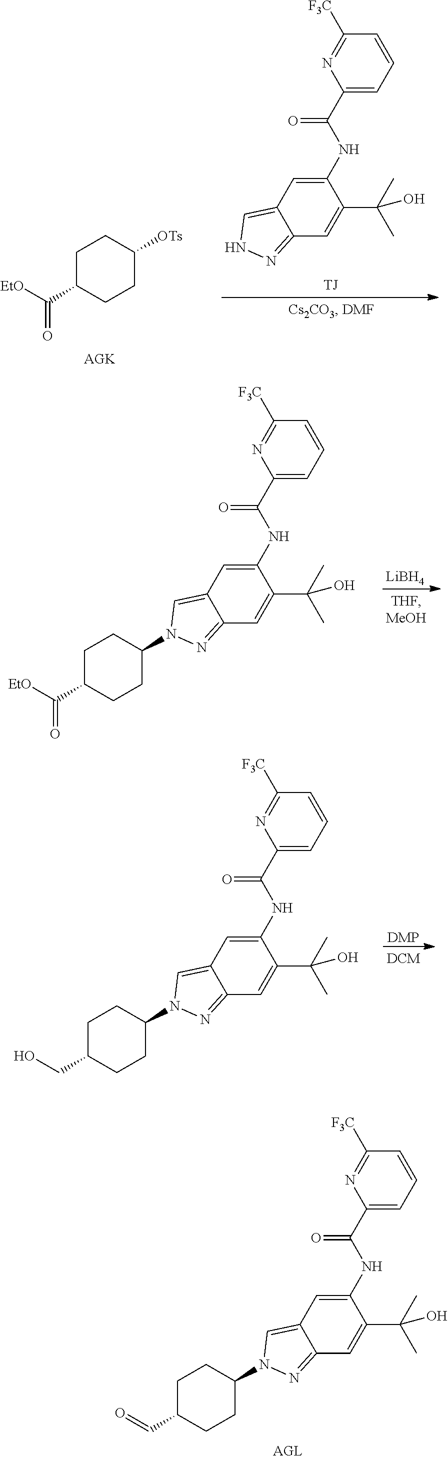

### Step 1—Ethyl 4-[6-(1-hydroxy-1-methyl-ethyl)-5-[[6-(trifluoromethyl)pyridine-2-carbonyl]amino]indazol-2-yl]cyclohexanecarboxylate

To a mixture of N-[6-(1-hydroxy-1-methyl-ethyl)-2H-indazol-5-yl]-6-(trifluoromethyl)pyridine-2-carboxamide (1.30 g, 3.57 mmol, Intermediate TJ), ethyl 4-(p-tolylsulfonyloxy)cyclohexane carboxylate (2.33 g, 7.14 mmol, Intermediate AGK) and Cs₂CO₃ (2.33 g, 7.14 mmol) in DMF (20 mL) was stirred at 80° C. for 16 hours. To the mixture was added ethyl 4-(p-tolylsulfonyloxy)cyclohexanecarboxylate (2.33 g, 7.14 mmol) and Cs₂CO₃ (2.33 g, 7.14 mmol) at 15° C. The mixture was stirred at 80° C. for 16 hours. On completion, after cooled to 15° C., the mixtures of two batches were combined, diluted with water (100 mL), and extracted with EA (3×60 mL). The organic layer was washed with brine (20 mL), dried over Na₂SO₄, filtered and concentrated in vacuo. The residue was purified by reverse phase flash and prep-HPLC (column: Phenomenex Synergi Max-RP 150*50 mm*10 um; mobile phase: [water (0.225% FA)-ACN]; B %: 52%-82%, 11 min) to give the title compound (530 mg, 14% yield) as white solid. ¹H NMR (400 MHz, CDCl₃) δ 12.28 (s, 1H), 8.87 (s, 1H), 8.50 (d, J=7.6 Hz, 1H), 8.10 (t, J=8.0 Hz, 1H), 7.92 (s, 1H), 7.84 (d, J=7.6 Hz, 1H), 7.74 (s, 1H), 4.43-4.35 (m, 1H), 4.17 (q, J=7.2 Hz, 2H), 2.48-2.40 (m, 1H), 2.36-2.34 (m, 2H), 2.28-2.19 (m, 3H), 2.10-1.97 (m, 2H), 1.81 (s, 6H), 1.76-1.64 (m, 2H), 1.29 (t, J=7.2 Hz, 3H).

### Step 2—N-[2-[4-(hydroxymethyl)cyclohexyl]-6-(1-hydroxy-1-methyl-ethyl)indazol-5-yl]-6-(trifluoro methyl)pyridine-2-carboxamide

To a solution of ethyl 4-[6-(1-hydroxy-1-methyl-ethyl)-5-[[6-(trifluoromethyl)pyridine-2-carbonyl]amino]indazol-2-yl]cyclohexanecarboxylate (200 mg, 385 umol) in THF (3 mL) and MeOH (0.4 mL) was added LiBH₄ (21.0 mg, 964 umol) at 0° C. The mixture was stirred at 50° C. for 1 hour. On completion, the reaction was quenched with sat. aq. NH₄Cl (5 mL). The mixture was diluted with water (40 mL), then extracted with EA (3×20 mL). The combined organic layer was washed with brine (10 mL), dried over Na₂SO₄, filtered and concentrated in vacuo to give the title compound (180 mg, 98% yield) as light yellow solid. ¹H NMR (400 MHz, DMSO-d₆) δ 12.35 (s, 1H), 8.71 (s, 1H), 8.48-8.42 (m, 1H), 8.39-8.34 (m, 2H), 8.16 (d, J=7.6 Hz, 1H), 7.58 (s, 1H), 6.51 (s, 1H), 5.93 (s, 1H), 4.46-4.35 (m, 1H), 3.29 (s, 2H), 2.19-2.10 (m, 2H), 1.92-1.89 (m, 4H), 1.62 (s, 6H), 1.25-1.11 (m, 3H).

### Step 3—N-[2-(4-formylcyclohexyl)-6-(1-hydroxy-1-methyl-ethyl)indazol-5-yl]-6-(trifluoromethyl) pyridine-2-carboxamide

To a solution of N-[2-[4-(hydroxymethyl)cyclohexyl]-6-(1-hydroxy-1-methyl-ethyl)indazol-5-yl]-6-(trifluoromethyl)pyridine-2-carboxamide (50.0 mg, 104 umol) in DCM (5 mL) was added DMP (89.0 mg, 209 umol) at 0° C. The mixture was stirred at 0-10° C. for 6 hours. On completion, the reaction was quenched with sat. aq. Na₂S₂O₃ (5 mL), and extracted with DCM (2×10 mL). The combined organic layer was washed with sat. aq. NaHCO₃ (5 mL), dried over Na₂SO₄, filtered and concentrated in vacuo to give the title compound (49.0 mg, 98% yield) as light yellow solid. LC-MS (ESI⁺) m/z 475.2 (M+H)⁺.

### 3-(4-Methoxybenzyl)dihydropyrimidine-2,4(1H,3H)-dione (Intermediate BRW)

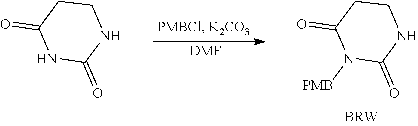

To a mixture of dihydropyrimidine-2,4(1H,3H)-dione (10.0 g, 87.6 mmol, CAS #504-07-4) in DMF (100 mL) was added PMB-Cl (13.7 g, 87.6 mmol, 11.9 mL), Cs₂CO₃ (28.5 g, 87.6 mmol) at 25° C. Then the mixture was stirred at 50° C. for 3 hours. On completion, the reaction mixture was quenched with of water (100 mL), and extracted with EtOAc (3×50 mL). The organic layer was dried over anhydrous Na₂SO₄, filtered and concentrated in vacuo. The crude product was purified by re-crystallization from EA/PE (20 mL, v/v=1/1) at 25° C. to give the title compound (9.40 g, 45% yield) as a white solid. ¹H NMR (400 MHz, CDCl₃) δ 7.81 (s, 1H), 7.18 (d, J=8.4 Hz, 2H), 6.83 (d, J=8.4 Hz, 2H), 4.72 (s, 2H), 3.72 (s, 3H), 3.23-3.20 (m, 2H), 2.63 (t, J=6.8 Hz, 2H).

### 1-(7-Chloroisoquinolin-4-yl)dihydropyrimidine-2,4(1H,3H)-dione (Intermediate BRX)

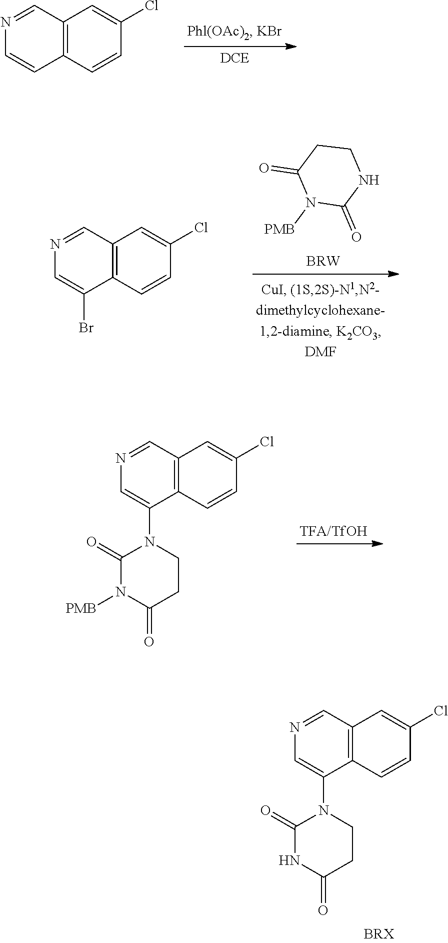

### Step 1—4-Bromo-7-chloroisoquinoline

To a solution of 7-chloroisoquinoline (5.00 g, 30.5 mmol, CAS #34784-06-0) in DCE (50 mL) was added PhI(OAc)₂ (14.7 g, 45.8 mmol) and KBr (18.1 g, 152 mmol) and the mixture was stirred at 50° C. for 16 hours. On completion, the mixture was poured into water (100 mL), and extracted with EA (300 mL). The organic layer was washed with brine (2×100 mL), dried over anhydrous Na₂SO₄, filtered and concentrated in vacuo. The mixture was purified by silica gel column to give the title compound (5.50 g, 65% yield) as a white solid. ¹H NMR (400 MHz, CDCl₃) δ 9.13 (s, 1H), 8.74 (s, 1H), 8.13 (d, J=9.2 Hz, 1H), 7.99 (d, J=2.0 Hz, 1H), 7.77 (dd, J=2.0, 9.2 Hz, 1H).

### Step 2—1-(7-Chloro-4-isoquinolyl)-3-[(4-methoxyphenyl)methyl]hexahydropyrimidine-2,4-dione

To a solution of 4-bromo-7-chloroisoquinoline (2.00 g, 8.25 mmol) and 3-(4-methoxybenzyl) dihydropyrimidine-2,4(1H,3H)-dione (1.93 g, 8.25 mmol, Intermediate BRW) in DMF (20 mL) was added (1S,2S)-N1,N2-dimethylcyclohexane-1,2-diamine (234 mg, 1.65 mmol), CuI (314 mg, 1.65 mmol) and K₂CO₃ (3.42 g, 24.7 mmol). Then the mixture was stirred at 100° C. for 16 hours under N₂. On completion, the reaction solution was diluted with water (100 mL) and then extracted with ethyl acetate (3×100 mL). The combined organic layers were washed with brine (2×100 mL), dried over anhydrous sodium sulfate, filtered and concentrated under vacuum. The mixture was purified by reversed phase flash: (C18, 10% to 40% MeCN in H₂O, contained 0.1% FA in H₂O) to give the title compound (200 mg, 5% yield) as a light yellow solid. ¹H NMR (400 MHz, DMSO-d₆) δ 9.31 (s, 1H), 8.60 (s, 1H), 8.39 (d, J=2.0 Hz, 1H), 8.00 (d, J=9.2 Hz, 1H), 7.83 (dd, J=2.0, 8.8 Hz, 1H), 7.25 (d, J=8.8 Hz, 2H), 6.90-6.86 (m, 2H), 4.83 (s, 2H), 4.00-3.94 (m, 1H), 3.79-3.76 (m, 1H), 3.73 (s, 3H), 3.19-3.11 (m, 1H), 2.99-2.92 (m, 1H).

### Step 3—1-(7-Chloroisoquinolin-4-yl)dihydropyrimidine-2,4(1H,3H)-dione

1-(7-Chloroisoquinolin-4-yl)-3-(4-methoxybenzyl)dihydropyrimidine-2,4(1H,3H)-dione (50.0 mg, 126 umol) was added into TFA (0.5 mL) and TfOH (0.01 mL) and the mixture was stirred at 60° C. for 2 hours. On completion, the reaction solution was diluted with water (5 mL) and then extracted with ethyl acetate (3×5 mL). The combined organic layers were washed with brine (2×5 mL), dried over anhydrous sodium sulfate, filtered and concentrated in vacuo. The residue was further purified by prep-HPLC (Column: [Phenomenex luna C18, 150 mm*25 mm*10 um]; mobile phase: (water (0.225% FA)-MeCN, MeCN%: 8%-38%); 11 min) to give the title compound (5.18 mg, 14% yield) as a yellow solid. ¹H NMR (400 MHz, DMSO-d₆) δ 10.56 (s, 1H), 9.31 (s, 1H), 8.59 (s, 1H), 8.38 (d, J=2.0 Hz, 1H), 8.06 (d, J=9.2 Hz, 1H), 7.84 (dd, J=2.4, 8.8 Hz, 1H), 4.00-3.93 (m, 1H), 3.75-3.69 (m, 1H), 3.02-2.94 (m, 1H), 2.78-2.71 (m, 1H). LC-MS (ESI⁺) m/z 275.9 (M+H)⁺.

### 1-(7-(Piperidin-4-yl)isoquinolin-4-yl)dihydropyrimidine-2,4(1H,3H)-dione (Intermediate BRY

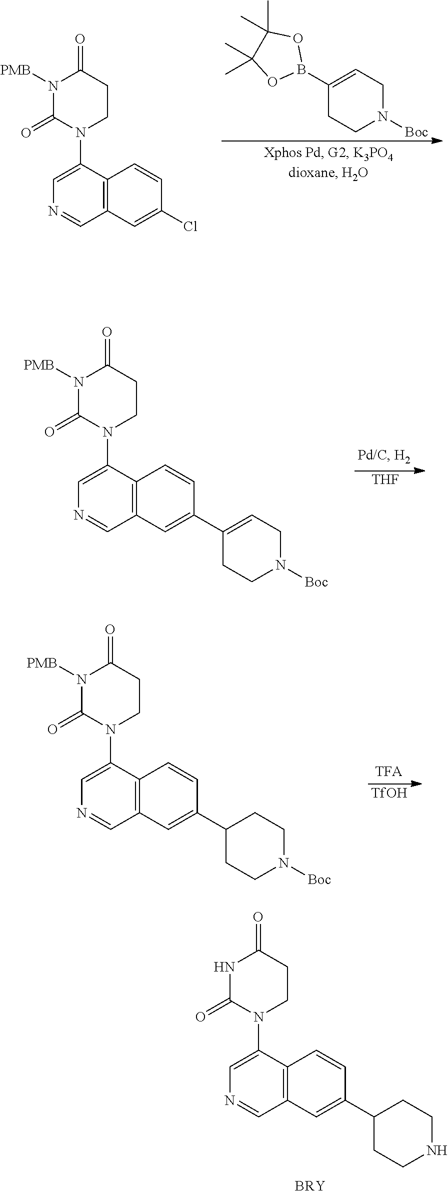

### Step 1—Tert-butyl 4-(4-(3-(4-methoxybenzyl)-2,4-dioxotetrahydropyrimidin-1(2H)-yl)isoquinolin-7-yl)-5,6-dihydropyridine-1(2H)-carboxylate

To a solution of 1-(7-chloroisoquinolin-4-yl)-3-(4-methoxybenzyl)dihydropyrimidine-2,4(1H,3H)-dione (150 mg, 378 umol, synthesized via Steps 1-2 of Intermediate BRX) and tert-butyl 4-(4,4,5,5-tetramethyl-1,3,2-dioxaborolan-2-yl)-5,6-dihydropyridine-1(2H)-carboxylate (140 mg, 454 umol, CAS #286961-14-6) in dioxane (2.0 mL) and water (0.2 mL) was added Xphos Pd G₂ (29.8 mg, 37.8 umol) and K₃PO₄ (160 mg, 757 umol). Then the mixture was stirred at 80° C. for 6 hours. On completion, the reaction solution was diluted with water (20 mL) and then extracted with ethyl acetate (3×10 mL). The combined organic layers were washed with brine (2×10 mL), dried over anhydrous sodium sulfate, filtered and concentrated under vacuum. The residue was purified by prep-TLC to afford the title compound (170 mg, 67% yield) as a brown oil. LC-MS (ESI⁺) m/z 543.4 (M+H)⁺.

### Step 2—Tert-butyl 4-(4-(3-(4-methoxybenzyl)-2,4-dioxotetrahydropyrimidin-1(2H)-yl)isoquinolin-7-yl)piperidine-1-carboxylate

To a solution of tert-butyl 4-(4-(3-(4-methoxybenzyl)-2,4-dioxotetrahydropyrimidin-1(2H)-yl) isoquinolin-7-yl)-5,6-dihydropyridine-1(2H)-carboxylate (160 mg, 294 umol) in THF (20 mL) was added Pd/C (30 mg, 294 umol, 10 wt %) under N₂. The mixture was stirred at 20° C. for 1 hour under H₂ balloon (15 psi). On completion, the mixture was filtered through celite, then washed with THF (50 mL). The filtrate was concentrated in vacuo to afford the title compound (130 mg, 72% yield) as brown oil. ¹H NMR (400 MHz, DMSO-d₆) δ 9.26 (s, 1H), 8.49 (s, 1H), 8.06 (s, 1H), 7.85 (d, J=8.4 Hz, 1H), 7.75 (dd, J=1.6, 8.8 Hz, 1H), 7.25 (d, J=8.8 Hz, 2H), 6.90-6.86 (m, 2H), 4.83 (s, 2H), 4.13 (d, J=11.0 Hz, 2H), 3.94-3.91 (m, 1H), 3.73 (s, 3H), 3.66-3.54 (m, 4H), 3.15-3.08 (m, 1H), 3.02-2.97 (m, 1H), 1.88-1.85 (m, 2H), 1.68-1.57 (m, 2H), 1.35 (s, 9H); LC-MS (ESI⁺) m/z 545.2 (M+H)⁺.

### Step 3—1-(7-(Piperidin-4-yl)isoquinolin-4-yl)dihydropyrimidine-2,4(1H,3H)-dione

A solution of tert-butyl 4-(4-(3-(4-methoxybenzyl)-2,4-dioxotetrahydropyrimidin-1(2H)-yl) isoquinolin-7-yl)piperidine-1-carboxylate (40.0 mg, 73.4 umol) in TFA (1.0 mL) and TfOH (0.05 mL) was stirred at 70° C. for 3 hours. On completion, the residue was concentrated in vacuo. The residue was purified by prep-HPLC (column: Phenomenex luna C18, 150 mm*25 mm*10 um; mobile phase: [water (0.225% FA)-MeCN]; B %: 1%-15%, 11.5 min), and then further purified by Prep-HPLC (column: Waters xbridge, 150 mm*25 mm*10 um; mobile phase: [water (10 mM NH₄HCO₃)-MeCN]; B %: 0%-26%, 11 min) to give the title compound (1.03 mg, 4% yield) as a white solid. ¹H NMR (DMSO-d₆, 400 Hz) δ 10.53 (s, 1H), 9.26 (s, 1H), 8.48 (s, 1H), 8.01 (s, 1H), 7.92 (d, J=8.8 Hz, 1H), 7.77-7.74 (m, 1H), 3.96-3.89 (m, 1H), 3.75-3.69 (m, 1H), 3.09 (d, J=12.0 Hz, 2H), 3.00-2.72 (m, 4H), 2.65-2.62 (m, 2H), 1.80 (d, J=12 Hz, 2H), 1.68-1.58 (m, 2H); LC-MS (ESI⁺) m/z 325.0 (M+H)⁺.

### 1-(7-(4-(Methylamino)piperidin-1-yl)isoquinolin-4-yl)dihydropyrimidine-2,4(1H,3H)-dione (Intermediate BRZ)

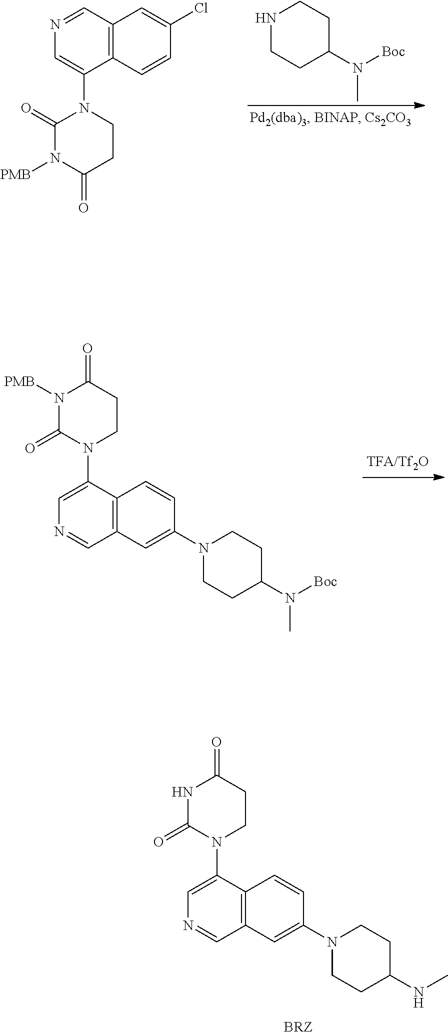

### Step 1—Tert-butyl (1-(4-(3-(4-methoxybenzyl)-2,4-dioxotetrahydropyrimidin-1(2H)-yl)isoquinolin-7-yl)piperidin-4-yl)(methyl)carbamate

To a solution of 1-(7-chloroisoquinolin-4-yl)-3-(4-methoxybenzyl)dihydropyrimidine-2,4(1H,3H)-dione (200 mg, 505 umol, synthesized via Steps 1-2 of Intermediate BRX) and tert-butyl methyl (piperidin-4-yl)carbamate (108 mg, 505 umol, CAS #108612-54-0) in dioxane (2 mL) was added Pd₂(dba)₃ (92.5 mg, 101 umol), BINAP (125 mg, 202 umol) and Cs₂CO₃ (329 mg, 1.01 mmol). Then the mixture was stirred at 100° C. for 12 hours under N₂. On completion, the mixture was poured into water (20 mL), then the mixture was extracted with EA (30 mL). The organic layer was washed with brine (2×10 mL), dried over anhydrous Na₂SO₄, filtered and concentrated in vacuo. The residue was purified by prep-HPLC (column: Phenomenex luna C18, 150 mm*25 mm*10 um; mobile phase: [water (0.225% FA)-MeCN]; B %: 33%-63%, 11.5 min) to give the title compound (50.0 mg, 15% yield) as a light yellow solid. ¹H NMR (400 MHz, DMSO-d₆) δ 9.07 (s, 1H), 8.27 (s, 1H), 7.75-7.69 (m, 1H), 7.66-7.61 (m, 1H), 7.45 (d, J=2.0 Hz, 1H), 7.25 (d, J=8.8 Hz, 2H), 6.91-6.84 (m, 2H), 4.83 (s, 2H), 4.01 (d, J=12.8 Hz, 2H), 3.92-3.86 (m, 1H), 3.73 (s, 3H), 3.15-3.06 (m, 2H), 2.99-2.82 (m, 4H), 2.68 (s, 3H), 1.86-1.74 (m, 2H), 1.71-1.61 (m, 2H), 1.41 (s, 9H).

### Step 2—1-(7-(4-(Methylamino)piperidin-1-yl)isoquinolin-4-yl)dihydropyrimidine-2,4(1H,3H)-dione

To a solution of tert-butyl (1-(4-(3-(4-methoxybenzyl)-2,4-dioxotetrahydropyrimidin-1(2H)-yl)isoquinolin-7-yl)piperidin-4-yl)(methyl)carbamate (20.0 mg, 122 umol) was added into TFA (0.5 mL) and TfOH (0.01 mL). The solution was stirred at 60° C. for 2 hours. On completion, the mixture was concentrated in vacuo. The residue was adjusted with triethylamine to pH=7. The mixture was purified by reversed phase flash (column: Phenomenex luna C18, 150 mm*25 mm*10 um; mobile phase: [water (0.225% FA)-MeCN]; B %: 1%-20%, 11.5 min) and further purified by Prep-HPLC (column: Waters xbridge, 150 mm*25 mm*10 um; mobile phase: [water (10 mM NH₄HCO₃)-MeCN]; B %: 0%-15%, 11 min) to give the title compound (1.17 mg, 94% yield) as a white solid. ¹H NMR (400 MHz, DMSO-d₆) δ ppm 10.49 (s, 1H), 9.05 (s, 1H), 8.25 (s, 1H), 7.81-7.74 (m, 1H), 7.65-7.63 (m, 1H), 7.41 (d, J=2.4 Hz, 1H), 3.93-3.79 (m, 3H), 3.71-3.69 (m, 1H), 2.95-2.89 (m, 4H), 2.88-2.71 (m, 2H), 2.32 (s, 3H), 1.94-1.91 (m, 2H), 1.41-1.36 (m, 2H). LC-MS (ESI⁺) m/z 354.2 (M+H)⁺.

### 1-[8-[4-(Methylamino)-1-piperidyl]-4-isoquinolyl]hexahydropyrimidine-2,4-dione (Intermediate BSA)

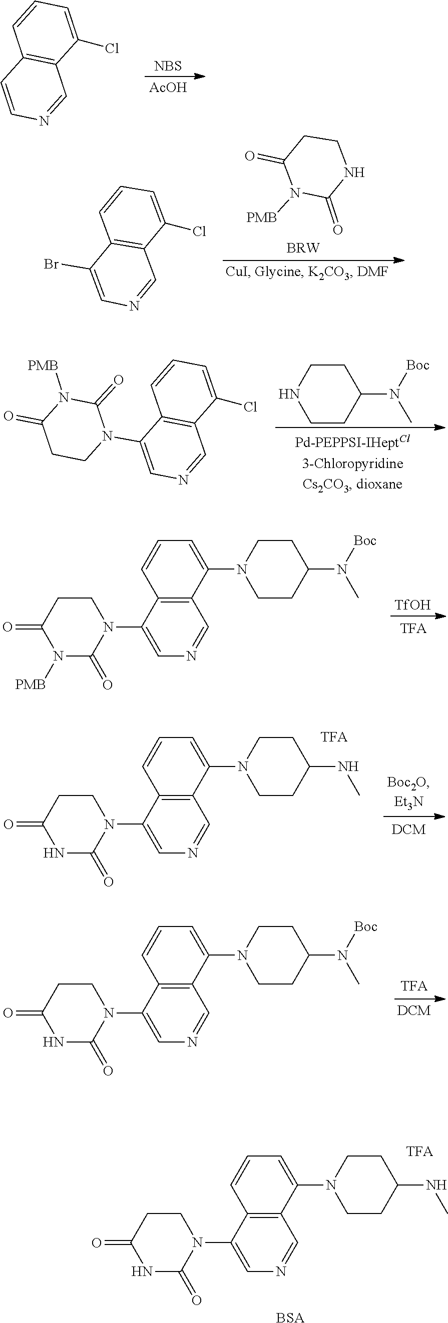

### Step 1—4-Bromo-8-chloro-isoquinoline

A mixture of 8-chloroisoquinoline (5.00 g, 30.5 mmol, CAS #34784-07-1), NBS (7.07 g, 39.7 mmol) in HOAc (50 mL) was degassed and purged with N₂ three times, and then the mixture was stirred at 50° C. for 40 minutes under N₂ atmosphere. On completion, the reaction mixture was neutralized with 15% NaOH (20 mL) and the mixture was extracted with EA (3×20 mL). The combined organic layers were washed with water (50 mL), dried over anhydrous Na₂SO₄, filtered and concentrated in vacuo to give a residue. Then the residue was purified by column chromatography to give the title compound (400 mg, 73.90% yield) as yellow solid. ¹H NMR (400 MHz, CDCl₃-d) δ 9.58 (s, 1H), 8.79 (s, 1H), 8.12-8.05 (m, 1H), 7.73-7.66 (m, 2H).

### Step 2—1-(8-Chloro-4-isoquinolyl)-3-[(4-methoxyphenyl)methyl]hexahydropyrimidine-2,4-dione

To a solution of 4-bromo-8-chloro-isoquinoline (200 mg, 824 umol) and 3-[(4-methoxyphenyl)methyl]hexahydropyrimidine-2,4-dione (231 mg, 989 umol, Intermediate BRW) in DMF (3 mL) was added CuI (47.1 mg, 247 umol), K₂CO₃ (227 mg, 1.65 mmol) and 2-aminoacetic acid (18.5 mg, 247 umol). Then the mixture was purged with N₂ three times and stirred at 140° C. for 8 hours. On completion, the mixture was filtrated, diluted with water (100 mL) and extracted with EA (5×80 mL). The combined organic phase was dried with anhydrous Na₂SO₄, filtered and concentrated in vacuo to give the residue. Then the residue was purified by reversed-phase HPLC (0.10% FA) to give the title compound (99.2 mg, 30.41% yield) as yellow solid. ¹H NMR (400 MHz, DMSO-d₆) δ 9.56 (s, 1H), 8.72 (s, 1H), 7.98 (d, J=8.4 Hz, 1H), 7.91-7.87 (m, 1H), 7.83-7.77 (m, 1H), 7.29-7.23 (m, 2H), 6.91-6.84 (m, 2H), 4.84 (s, 2H), 4.01-3.94 (m, 1H), 3.80-3.75 (m, 1H), 3.73-3.71 (m, 3H), 3.20-3.12 (m, 1H), 3.01-2.93 (m, 1H). LC-MS (ESI⁺) m/z 396.0 (M+H)⁺.

### Step 3—Tert-butyl N-[1-[4-[3-[(4-methoxyphenyl)methyl]-2,4-dioxo-hexahydropyrimidin-1-yl]-8-isoquinolyl]-4-piperidyl]-N-methyl-carbamate

To a solution of 1-(8-chloro-4-isoquinolyl)-3-[(4-methoxyphenyl)methyl]hexahydropyrimidine-2,4-dione (200 mg, 505 umol) and tert-butyl N-methyl-N-(4-piperidyl)carbamate (119 mg, 555 umol, CAS #108612-54-0) in dioxane (4 mL) was added Cs₂CO₃ (329 mg, 1.01 mmol) and Pd-PEPPSI-IHept^{Cl}3-Chloropyridine (49.1 mg, 50.5 umol), then the mixture was stirred at 80° C. for 8 hours. On completion, the mixture was filtered, diluted with water (20 mL) and extracted with EA (4×10 mL). The extract was dried over anhydrous Na₂SO₄, filtered and concentrated in vacuo to give the title compound (216 mg, 74.52% yield) as yellow solid. ¹H NMR (400 MHz, DMSO-d₆) δ 9.45 (s, 1H), 8.53 (s, 1H), 7.72-7.66 (m, 1H), 7.54 (d, J=8.4 Hz, 1H), 7.31-7.24 (m, 3H), 6.88 (d, J=8.8 Hz, 2H), 4.83 (s, 2H), 3.93-3.87 (m, 1H), 3.78-3.71 (m, 4H), 3.50-3.42 (m, 2H), 3.17-3.08 (m, 1H), 2.99-2.96 (m, 1H), 2.81 (s, 3H), 2.18-2.03 (m, 2H), 1.75-1.68 (m, 2H), 1.43 (s, 9H), 0.88-0.70 (m, 3H); LC-MS (ESI⁺) m/z 574.3 (M+H)⁺.

### Step 4—1-[8-[4-(Methylamino)-1-piperidyl]-4-isoquinolyl]hexahydropyrimidine-2,4-dione

To a solution of tert-butyl N-[1-[4-[3-[(4-methoxyphenyl)methyl]-2,4-dioxo-hexahydropyrimidin-1-yl]-8-isoquinolyl]-4-piperidyl]-N-methyl-carbamate (206 mg, 359 umol) in TFA (0.5 mL) and TfOH (0.05 mL) was stirred at 70° C. for 1 hour. On completion, the mixture was concentrated in vacuo to give the title compound (100 mg, 78.80% yield, TFA) as yellow solid. LC-MS (ESI⁺) m/z 354.0 (M+H)⁺.

### Step 5—Tert-butyl N-[1-[4-(2,4-dioxohexahydropyrimidin-1-yl)-8-isoquinolyl]-4-piperidyl]-N-methyl-carbamate

To a solution of 1-[8-[4-(methylamino)-1-piperidyl]-4-isoquinolyl]hexahydropyrimidine-2,4-dione (100 mg, 282 umol) in DCM (1 mL) was added Et₃N (787 uL, 5.66 mmol) and Boc₂O (92.6 mg, 424 umol) at 0° C., then the mixture was stirred at 25° C. for 13 hours. On completion, the mixture was concentrated in vacuo to give the residue, then the residue was purified by reverse-phase (0.1% FA condition) to give the title compound (70.0 mg, 54.55% yield) as yellow solid. ¹H NMR (400 MHz, DMSO-d₆) δ 10.53 (s, 1H), 9.45 (s, 1H), 8.53 (s, 1H), 7.75-7.68 (m, 1H), 7.61 (d, J=8.4 Hz, 1H), 7.28 (d, J=7.2 Hz, 1H), 4.15-3.96 (m, 1H), 3.92-3.86 (m, 1H), 3.72-3.66 (m, 1H), 3.48-3.41 (m, 2H), 3.01-2.84 (m, 3H), 2.81 (s, 3H), 2.78-2.71 (m, 1H), 2.19-2.02 (m, 2H), 1.72-1.70 (m, 2H), 1.43 (s, 9H). LC-MS (ESI⁺) m/z 454.1 (M+H)⁺.

### Step 6—1-[8-[4-(Methylamino)-1-piperidyl]-4-isoquinolyl]hexahydropyrimidine-2,4-dione

To a solution of tert-butyl N-[1-[4-(2,4-dioxohexahydropyrimidin-1-yl)-8-isoquinolyl]-4-piperidyl]-N-methyl-carbamate (60 mg, 132 umol) in DCM (1 mL) was added TFA (0.5 mL, 6.75 mmol), then the mixture was stirred at 25° C. for 1 hour. On completion, the mixture was concentrated in vacuo to give the title compound (58.0 mg, 93.79% yield, TFA) as yellow solid. LC-MS (ESI⁺) m/z 354.0 (M+H)⁺.

### N-[6-cyclopropyl-2-(4-formylcyclohexyl)indazol-5-yl]-6-(trifluoromethyl)pyridine-2-carboxamide (Intermediate BSB)

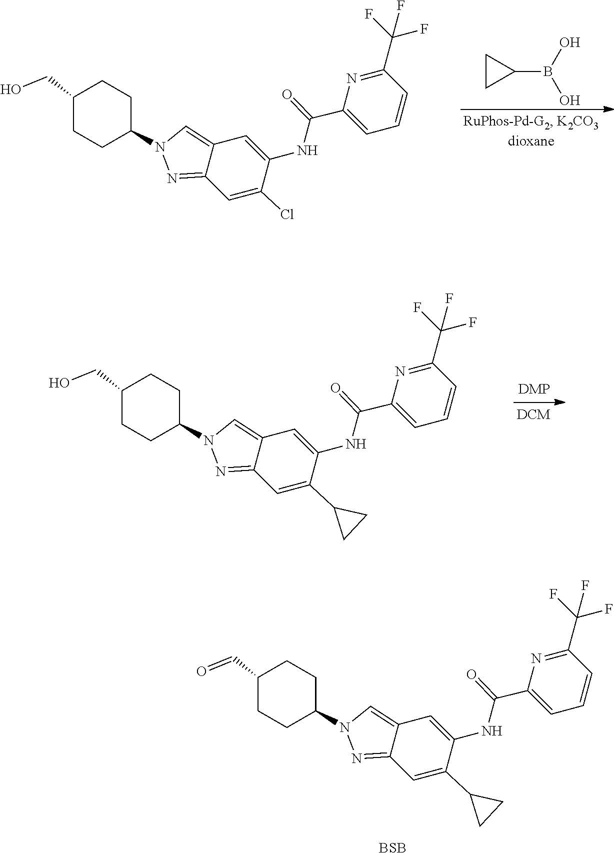 [SMILES: O=C(Nc1cc2cn([C@H]3CC[C@H](CO)CC3)nc2cc1Cl)c1cccc(C(F)(F)F)n1 ; OB(O)C1CC1 ; O=C(Nc1cc2cn([C@H]3CC[C@H](CO)CC3)nc2cc1C1CC1)c1cccc(C(F)(F)F)n1 ; O=C[C@H]1CC[C@H](n2cc3cc(NC(=O)c4cccc(C(F)(F)F)n4)c(C4CC4)cc3n2)CC1 ; "BSB"]

### Step 1—N-[6-cyclopropyl-2-[4-(hydroxymethyl)cyclohexyl]indazol-5-yl]-6-(trifluoromethyl)pyridine-2-carboxamide

To a solution of N-[6-chloro-2-[4-(hydroxymethyl)cyclohexyl]indazol-5-yl]-6-(trifluoromethyl) pyridine-2-carboxamide (100 mg, 220 umol, synthesized via Steps 1-3 of Intermediate BPQ) in dioxane (2.5 mL) was added cyclopropylboronic acid (37.9 mg, 441 umol, CAS #411235-57-9), K₂CO₃ (91.5 mg, 662 umol) and XPHOS-PD-G₂ (17.3 mg, 22.1 umol). The reaction mixture was stirred at 90° C. for 12 hrs under N₂ atmosphere. On completion, the reaction mixture was filtered and concentrated in vacuo to give a residue. The residue was purified by prep-HPLC (column: Phenomenex luna C18 150*25 mm*10 um; mobile phase: [water (0.225% FA)-ACN]; B %: 46%-76%, 10 min) to give the title compound (40 mg, 39% yield) as a white solid. ¹H NMR (400 MHz, DMSO-d₆) δ 10.74 (s, 1H), 8.63 (s, 1H), 8.52-8.45 (m, 1H), 8.45-8.34 (m, 2H), 8.22 (d, J=7.6 Hz, 1H), 7.50-7.45 (m, 1H), 4.48 (t, J=5.2 Hz, 1H), 4.45-4.33 (m, 1H), 3.30-3.25 (m, 2H), 2.20-2.08 (m, 2H), 2.08-1.98 (m, 1H), 1.96-1.80 (m, 4H), 1.53-1.42 (m, 1H), 1.16-1.06 (m, 3H), 0.88 (q, J=7.2 Hz, 1H), 0.82-0.72 (m, 2H); LC-MS (ESI⁺) m/z 459.0 (M+H)⁺.

### Step 2—N-[6-cyclopropyl-2-(4-formylcyclohexyl)indazol-5-yl]-6-(trifluoromethyl)pyridine-2-carboxamide

To a solution of N-[6-cyclopropyl-2-[4-(hydroxymethyl)cyclohexyl]indazol-5-yl]-6-(trifluoromethyl) pyridine-2-carboxamide (40 mg, 87.2 umol) in DCM (0.5 mL) was added DMP (48.1 mg, 113 umol). The reaction mixture was stirred at 25° C. for 2 hrs. On completion, the reaction mixture was quenched by saturated Na₂S₂O₃ (2 mL) and saturated NaHCO₃ (2 mL) at 25° C., and then the mixture was stirred for 15 minutes. The mixture was extracted with DCM (2×5 mL), then the combined organic layers was washed with saturated NaCl (2×10 mL). Then the combined organic layers was dried over Na₂SO₄, filtered and concentrated in vacuo to give title compound (30 mg, 75% yield) as yellow oil. ¹H NMR (400 MHz, DMSO-d₆) δ 10.83-10.66 (m, 1H), 9.64 (s, 1H), 8.64 (s, 1H), 8.52-8.45 (m, 1H), 8.44-8.34 (m, 2H), 8.22 (d, J=7.6 Hz, 1H), 7.52-7.45 (m, 1H), 4.55-4.34 (m, 1H), 3.31-3.22 (m, 1H), 2.23-2.08 (m, 3H), 2.05-1.90 (m, 3H), 1.44 (dd, J=3.2, 12.4 Hz, 1H), 1.23 (s, 1H), 1.15-1.07 (m, 2H), 0.87 (q, J=7.2 Hz, 1H), 0.81-0.72 (m, 2H); LC-MS (ESI⁺) m/z 457.3 (M+H)⁺.

### N-[2-(4-formylcyclohexyl)-6-methyl-indazol-5-yl]-6-(trifluoromethyl)pyridine-2-carboxamide (Intermediate BSC)

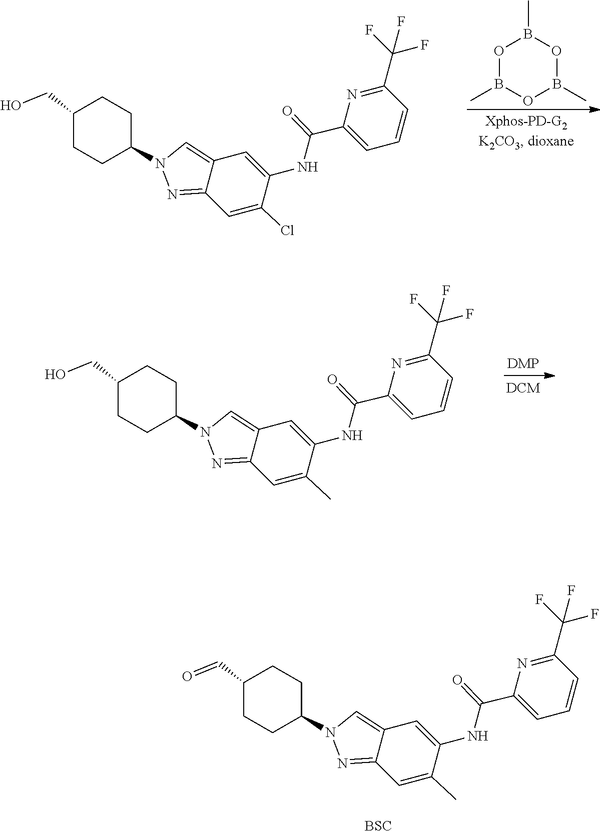

### Step 1—N-[2-[4-(hydroxymethyl)cyclohexyl]-6-methyl-indazol-5-yl]-6-(trifluoromethyl)pyridine-2-carboxamide

To a solution of N-[6-chloro-2-[4-(hydroxymethyl)cyclohexyl]indazol-5-yl]-6-(trifluoromethyl) pyridine-2-carboxamide (50.0 mg, 110 umol, via Steps 1-3 of Intermediate BPQ) in dioxane (1 mL) was added 2,4,6-trimethyl-1,3,5,2,4,6-trioxatriborinane (27.7 mg, 220 umol, CAS #823-96-1), K₂CO₃ (45.7 mg, 331 umol) and XPHOS-PD-G₂ (8.69 mg, 11.0 umol). The reaction mixture was stirred at 90° C. for 16 hrs under N₂ atmosphere. On completion, the reaction mixture was diluted with water (5 mL) and extracted with DCM (2×10 mL). The combined organic layer was dried over Na₂SO₄, filtered and concentrated in vacuo to the title compound (38.0 mg, 79% yield) as a white solid. ¹H NMR (400 MHz, DMSO-d₆) δ 10.12 (s, 1H), 8.46-8.34 (m, 3H), 8.23-8.17 (m, 2H), 7.53-7.46 (m, 1H), 4.53-4.34 (m, 2H), 3.29 (s, 2H), 2.40 (s, 3H), 2.17-2.12 (m, 2H), 1.94-1.86 (m, 4H), 1.52-1.45 (m, 1H), 1.17-1.13 (m, 2H); LC-MS (ESI⁺) m/z 433.3 (M+H)⁺.

### Step 2—N-[2-(4-formlylcyclohexyl)-6-methyl-indazol-5-yl]-6-(trifluoromethyl)pyridine-2-carboxamide

To a solution of N-[2-[4-(hydroxymethyl)cyclohexyl]-6-methyl-indazol-5-yl]-6-(trifluoromethyl) pyridine-2-carboxamide (38.0 mg, 87.8 umol) in DCM (0.5 mL) was added DMP (48.4 mg, 114 umol) and the mixture was stirred at 25° C. for 2 hrs. On completion, the reaction mixture was quenched with saturated Na₂S₂O₃ (2 mL) and saturated NaHCO₃ (2 mL) at 25° C., and then stirred for 15 minutes. The mixture was extracted with DCM (2×5 mL), then the combined organic layers was washed with saturated NaCl (2×10 mL). Then the combined organic layers was dried over Na₂SO₄, filtered and concentrated in vacuo to give the title compound (30.0 mg, 79% yield) as a yellow solid. ¹H NMR (400 MHz, DMSO-d₆) δ 10.13 (s, 1H), 9.65 (s, 1H), 8.49-8.32 (m, 3H), 8.27-8.14 (m, 2H), 7.51 (s, 1H), 4.55-4.33 (m, 1H), 2.40 (s, 3H), 2.21 (dd, J=2.4, 12.0 Hz, 2H), 2.14-2.08 (m, 2H), 1.98 (dd, J=3.2, 12.4 Hz, 2H), 1.45 (dd, J=3.2, 12.8 Hz, 2H), 1.25 (s, 1H); LC-MS (ESI⁺) m/z 431.2 (M+H)⁺.

### 1-(8-Chloro-4-isoquinolyl)hexahydropyrimidine-2,4-dione (Intermediate BSL)

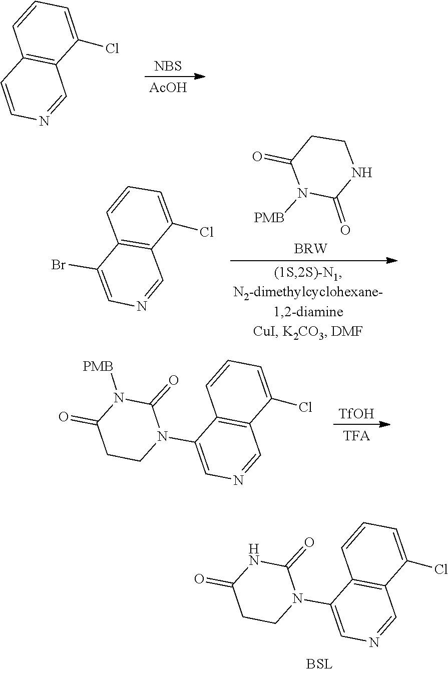

### Step 1—4-Bromo-8-chloro-isoquinoline

To a solution of 8-chloroisoquinoline (5.00 g, 30.5 mmol, CAS #34784-07-1) in AcOH (50 mL) was added NBS (7.07 g, 39.7 mmol), then the reaction mixture was stirred at 50° C. for 40 min. On completion, the reaction mixture was diluted with water (100 mL), then extracted with EA (3×80 mL). The combined organic layer was basified with NaHCO₃ until the pH=6-7, then the mixture was extracted with EA (2×60 mL). The combined organic layers was dried over Na₂SO₄, filtered and concentrated in vacuo to give a residue. The residue was purified by column chromatography (SiO₂, PE:EA=100:1 to PE:EA=50:1, PE:EA=10:1, P1:R_{f}=0.74) to give the title compound (1.00 g, 37% yield) as yellow solid. ¹HNMR (400 MHz, CDCl₃) δ 9.56 (s, 1H), 8.78 (s, 1H), 8.10-8.03 (m, 1H), 7.73-7.64 (m, 2H). LC-MS (ESI⁺) m/z 241.9 (M+H)⁺.

### Step 2—1-(8-Chloro-4-isoquinolyl)-3-[(4-methoxyphenyl)methyl]hexahydropyrimidine-2,4-dione

To a solution of 4-bromo-8-chloro-isoquinoline (100 mg, 412 umol) and 3-[(4-methoxyphenyl)methyl]hexahydropyrimidine-2,4-dione (96.6 mg, 412.37 umol, Intermediate BRW) in DMF (1 mL) was added CuI (7.85 mg, 41.2 umol), (1S,2S)-N₁,N₂-dimethylcyclohexane-1,2-diamine (5.87 mg, 41.2 umol) and K₃PO₄ (175 mg, 824 umol), then the mixture was stirred at 110° C. for 8 hr. On completion, the reaction mixture was filtered and concentrated in vacuo to give the residue. The residue was diluted with water (50 mL) and extracted with EA (5×30 mL). Then the combined organic layers was dried over anhydrous Na₂SO₄, filtered and concentrated in vacuo to give a residue. The residue was purified by reverse-phase (0.1% FA) to give the title compound (15 mg, 3.06% yield) as yellow solid. ¹H NMR (400 MHz, DMSO-d₆) δ 9.89-9.56 (br s, 1H), 8.59 (br s, 1H), 7.73-7.68 (m, 1H), 7.64 (t, J=8.0 Hz, 1H), 7.60-7.55 (m, 1H), 7.43 (d, J=8.4 Hz, 2H), 6.85 (d, J=8.4 Hz, 2H), 5.00 (s, 2H), 3.95-3.86 (m, 1H), 3.80 (s, 3H), 3.78-3.69 (m, 1H), 3.07-2.99 (m, 2H); LC-MS (ESI⁺) m/z 396.1 (M+H)⁺.

### Step 3—1-(8-Chloro-4-isoquinolyl)hexahydropyrimidine-2,4-dione

To a solution of 1-(8-chloro-4-isoquinolyl)-3-[(4-methoxyphenyl)methyl]hexahydropyrimidine-2,4-dione (40.0 mg, 101 umol) in TFA (0.49 mL) and TfOH (0.01 mL), then the mixture was stirred at 60° C. for 2 hours. On completion, the mixture was concentrated to give the residue and purified by prep-HPLC (0.10% FA) to give the title compound (3 mg, 10.77% yield) as yellow solid. ¹H NMR (400 MHz, DMSO-d₆) δ=10.59 (s, 1H), 9.56 (s, 1H), 8.71 (s, 1H), 8.03 (d, J=8.4 Hz, 1H), 7.92-7.87 (m, 1H), 7.85-7.78 (m, 1H), 4.00-3.93 (m, 1H), 3.75-3.69 (m 1H), 3.03-2.95 (m, 1H), 2.79-2.72 (m, 1H). LC-MS (ESI⁺) m/z 276.0 (M+H)⁺.

### 1-[8-(4-Piperidyl)-4-isoquinolyl]hexahydropyrimidine-2,4-dione (Intermediate BSN)

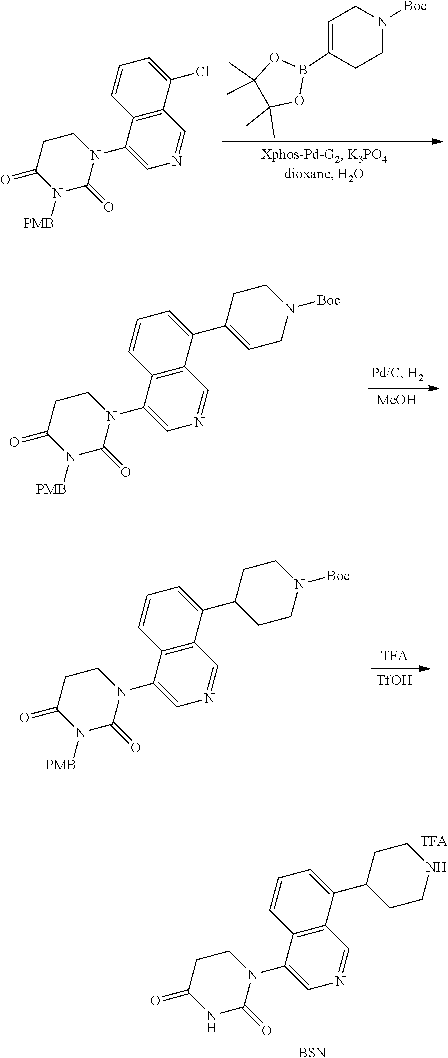

### Step 1—Tert-butyl 4-[4-[3-[(4-methoxyphenyl)methyl]-2,4-dioxo-hexahydropyrimidin-1-yl]-8-isoquinolyl]-3,6-dihydro-2H-pyridine-1-carboxylate

To a mixture of 1-(8-chloro-4-isoquinolyl)-3-[(4-methoxyphenyl)methyl]hexahydropyrimidine-2,4-dione (220 mg, 555 umol, synthesized via Steps 1-2 of Intermediate BSL), tert-butyl 4-(4,4,5,5-tetramethyl-1,3,2-dioxaborolan-2-yl)-3,6-dihydro-2H-pyridine-1-carboxylate (223 mg, 722 umol, CAS #286961-14-6) and K₃PO₄ (353 mg, 1.67 mmol) in dioxane (1 mL) and H₂O (0.05 mL) was added XPHOS-PD-G2 (43.7 mg, 55.5 umol). The reaction mixture was stirred at 80° C. for 2.5 hours. On completion, the reaction mixture was filtered and concentrated in vacuo. The residue was purified by reverse phase (0.1% FA condition) to give the title compound (240 mg, 79% yield) as light yellow solid. ¹H NMR (400 MHz, DMSO-d₆) δ 9.35 (s, 1H), 8.59 (s, 1H), 7.91-7.83 (m, 1H), 7.79 (dd, J=7.2, 8.4 Hz, 1H), 7.55 (d, J=7.2 Hz, 1H), 7.26 (d, J=8.4 Hz, 2H), 6.91-6.85 (m, 2H), 5.87 (s, 1H), 4.84 (s, 2H), 4.09 (s, 2H), 3.94 (ddd, J=5.2, 9.6, 12.0 Hz, 1H), 3.81-3.74 (m, 1H), 3.73 (s, 3H), 3.68 (t, J=5.4 Hz, 2H), 3.21-3.10 (m, 1H), 3.02-2.92 (m, 1H), 2.49-2.44 (m, 2H), 1.47 (s, 9H).

### Step 2—Tert-butyl 4-[4-[3-[(4-methoxyphenyl)methyl]-2,4-dioxo-hexahydropyrimidin-1-yl]-8-isoquinolyl]piperidine-1-carboxylate

To a mixture of tert-butyl 4-[4-[3-[(4-methoxyphenyl)methyl]-2,4-dioxo-hexahydropyrimidin-1-yl]-8-isoquinolyl]-3,6-dihydro-2H-pyridine-1-carboxylate (230 mg, 423 umol) in MeOH (10 mL) was added Pd/C (100 mg, 10 wt %). The reaction mixture was stirred at 40° C. for 3 hours under H₂ (15 Psi) atmosphere. On completion, the reaction mixture was filtered and concentrated in vacuo to give the title compound (220 mg, 95% yield) as white solid. ¹H NMR (400 MHz, DMSO-d₆) δ 9.68 (s, 1H), 8.58 (s, 1H), 7.82-7.72 (m, 2H), 7.62 (d, J=6.8 Hz, 1H), 7.28-7.23 (m, 2H), 6.90-6.86 (m, 2H), 4.83 (s, 2H), 4.14 (d, J=9.6 Hz, 2H), 3.92 (ddd, J=5.2, 9.6, 12.4 Hz, 1H), 3.83 (t, J=11.6 Hz, 1H), 3.78-3.74 (m, 1H), 3.73 (s, 3H), 3.15-2.92 (m, 4H), 1.95-1.85 (m, 2H), 1.74-1.60 (m, 2H), 1.44 (s, 9H).

### Step 3—1-[8-(4-Piperidyl)-4-isoquinolyl]hexahydropyrimidine-2,4-dione

To a mixture of tert-butyl 4-[4-[3-[(4-methoxyphenyl)methyl]-2,4-dioxo-hexahydropyrimidin-1-yl]-8-isoquinolyl]piperidine-1-carboxylate (210 mg, 385 umol) in TFA (3 mL) was added TfOH (0.2 mL). The reaction mixture was stirred at 70° C. for 2 hr. On completion, the reaction mixture was concentrated in vacuo. The residue was purified by reverse phase (0.1% FA condition) to give the title compound (200 mg, 98% yield, TFA) as red oil. LC-MS (ESI⁺) m/z 325.0 (M+H)⁺.

### 3-(4-Bromo-5-fluoro-3-methyl-2-oxo-benzimidazol-1-yl)piperidine-2,6-dione (Intermediate BPW)

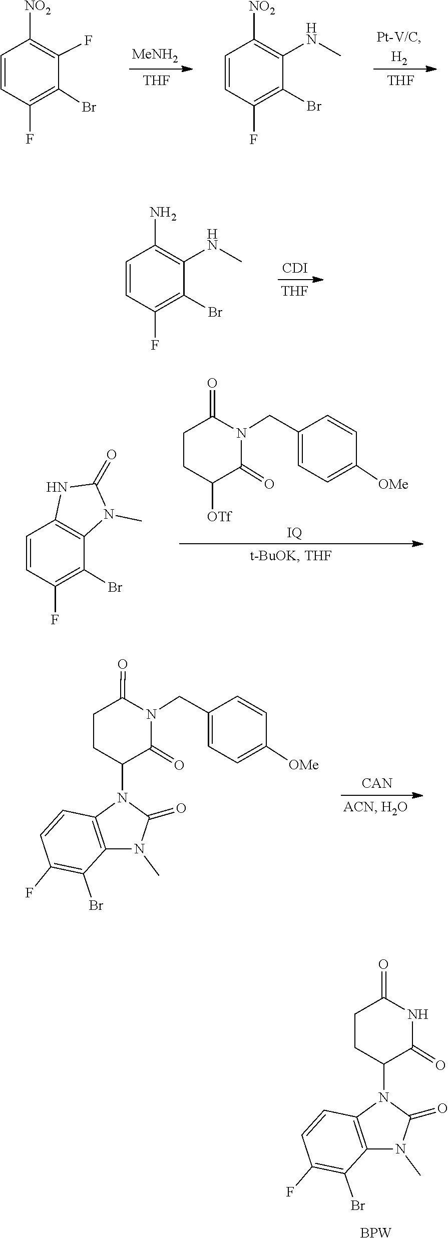

### Step 1—2-Bromo-3-fluoro-N-methyl-6-nitro-aniline

A solution of 2-bromo-1,3-difluoro-4-nitro-benzene (10.0 g, 42.0 mmol, CAS #103977-78-2) in THF (100 mL) saturated with MeNH₂ (2.00 M, 31.5 mL) was stirred at 60° C. for 5 hrs in a sealed tube. Additional MeNH₂ (2.00 M, 10.5 mL) was added, and the mixture was stirred at 60° C. for 2 hrs in a sealed tube. On completion, the mixture was concentrated in vacuo. The mixture was purified by silica gel column (PE) to give the title compound (10.3 g, 98% yield) as yellow solid. ¹H NMR (400 MHz, DMSO-d₆) δ 7.90 (dd, J=6.4, 9.6 Hz, 1H), 6.78 (dd, J=7.6, 9.6 Hz, 2H), 2.76 (d, J=5.2 Hz, 3H), LC-MS (ESI⁺) m/z 248.9 (M+H)⁺.

### Step 2—3-Bromo-4-fluoro-N2-methyl-benzene-1,2-diamine

To a solution of 2-bromo-3-fluoro-N-methyl-6-nitro-aniline (10.0 g, 40.1 mmol) in THF (100 mL) was added Pt-V/C (524 mg, 2.01 mmol) and the mixture was stirred at 25° C. for 16 hrs under H₂ (15 psi). On completion, the mixture was filtered and concentrated in vacuo to give the title compound (8.7 g, 98% yield) as yellow solid. ¹H NMR (400 MHz, DMSO-d₆) δ 6.75-6.65 (m, 1H), 6.60 (dd, J=6.0, 8.8 Hz, 1H), 4.82 (s, 2H), 3.91 (s, 1H), 2.62 (d, J=4.0 Hz, 3H), LC-MS (ESI⁺) m/z 221.1 (M+H)⁺.

### Step 3—4-Bromo-5-fluoro-3-methyl-1H-benzimidazol-2-one

To a solution of 3-bromo-4-fluoro-N2-methyl-benzene-1,2-diamine (8.70 g, 39.7 mmol) in ACN (120 mL) was added CDI (19.3 g, 119 mmol) and the mixture was stirred at 85° C. for 16 hrs. On completion, the mixture was concentrated in vacuo. The mixture was diluted with H₂O (300 mL), filtered and the filtrate was dried in vacuo to give the title compound (8.9 g, 91% yield) as gray solid, ¹H NMR (400 MHz, DMSO-d₆) δ 11.18 (s, 1H), 7.05-6.87 (m, 2H), 3.57 (s, 3H), LC-MS (ESI⁺) m/z 245.0 (M+H)⁺.

### Step 4—3-(4-Bromo-5-fluoro-3-methyl-2-oxo-benzimidazol-1-yl)-1-[(4-methoxyphenyl)methyl]piperidine-2,6-dione

To a solution of 4-bromo-5-fluoro-3-methyl-1H-benzimidazol-2-one (2.50 g, 10.2 mmol) in THF (30.0 mL) was added t-BuOK (2.06 g, 18.3 mmol) at −10° C. The mixture was stirred at −10° C. for 1 hr. Then a solution of [1-[(4-methoxyphenyl) methyl]-2,6-dioxo-3-piperidyl]trifluoromethanesulfonate (7.00 g, 18.3 mmol, Intermediate IQ) in THF (30.0 mL) was added to above solution and the mixture was stirred at 25° C. for 16 hrs. On completion, the mixture was quenched with saturated NH₄Cl solution (50 mL) and concentrated in vacuo to remove THF. The solution was then extracted with EA (3×40 mL), the organic layers were washed with brine (2×40 mL) dried over anhydrous Na₂SO₄, filtered and concentrated in vacuo. The mixture was purified by reverse phase: (0.1% FA) to give the title compound (3.5 g, 72% yield) as blue solid. ¹H NMR (400 MHz, DMSO-d₆) δ 7.23-7.18 (m, 2H), 7.12-7.00 (m, 2H), 6.87-6.83 (m, 2H), 5.61-5.56 (m, 1H), 4.86-4.71 (m, 2H), 3.72 (s, 3H), 3.65 (s, 3H), 3.12-2.91 (m, 1H), 2.87-2.69 (m, 2H), 2.09-2.00 (m, 1H), LC-MS (ESI⁺) m/z 478.2 (M+H)⁺.

### Step 5—3-(4-Bromo-5-fluoro-3-methyl-2-oxo-benzimidazol-1-yl)piperidine-2,6-dione

To a solution of 3-(4-bromo-5-fluoro-3-methyl-2-oxo-benzimidazol-1-yl)-1-[(4-methoxyphenyl) methyl]piperidine-2,6-dione (500 mg, 1.05 mmol) in ACN (20.0 mL) was added a solution of CAN (2.88 g, 5.25 mmol) in H₂O (5 mL) and the mixture was stirred at 25° C. for 16 hrs. On completion, the mixture was diluted with H₂O (20 mL) and extracted with EA (2×30 mL). The organic layers were washed with brine (2×20 mL), dried over anhydrous Na₂SO₄, filtered and concentrated in vacuo. The mixture was purified by prep-HPLC (column: 3_Phenomenex Luna C18 75*30 mm*3 um; mobile phase: [water (0.05% HCl)-ACN]; B %: 25%-45%, 7 min) to give the title compound (80 mg, 21% yield) as off-white solid. ¹H NMR (400 MHz, DMSO-d₆) δ 11.13 (s, 1H), 7.20-7.13 (m, 1H), 7.12-7.03 (m, 1H), 5.45-5.35 (m, 1H), 3.64 (s, 3H), 2.94-2.79 (m, 1H), 2.77-2.67 (m, 1H), 2.66-2.58 (m, 1H), 2.10-1.95 (m, 1H), LC-MS (ESI⁺) m/z 358.0 (M+H)⁺.

### 3-[5-Fluoro-3-methyl-2-oxo-4-(4-piperidyl)benzimidazol-1-yl]piperidine-2,6-dione (Intermediate BSP)

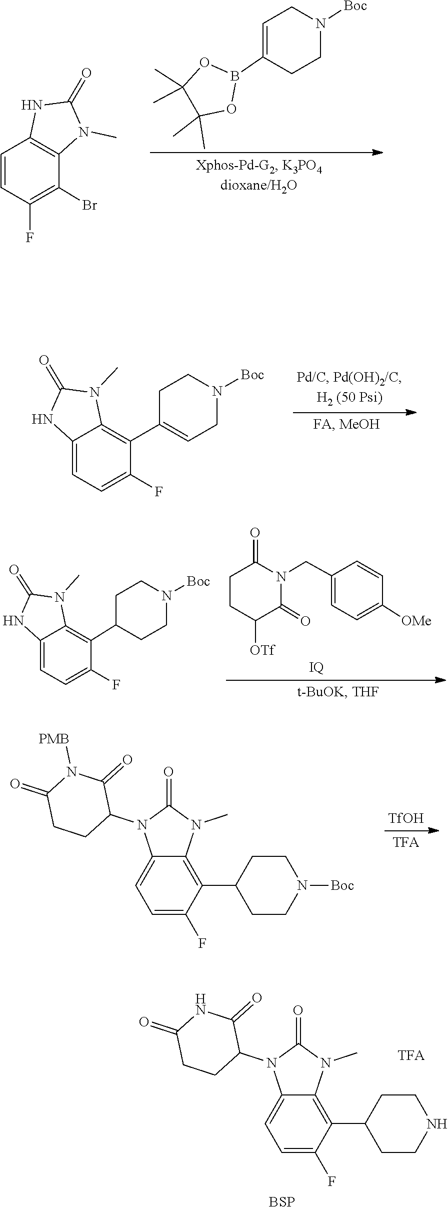

### Step 1—Tert-butyl 4-(5-fluoro-3-methyl-2-oxo-1H-benzimidazol-4-yl)-3,6-dihydro-2H-pyridine-1-carboxylate

To a mixture of 4-bromo-5-fluoro-3-methyl-1H-benzimidazol-2-one (0.50 g, 2.04 mmol, synthesized via Steps 1-3 of Intermediate BPW) and tert-butyl 4-(4,4,5,5-tetramethyl-1,3,2-dioxaborolan-2-yl)-3,6-dihydro-2H-pyridine-1-carboxylate (1.89 g, 6.12 mmol, CAS #286961-14-6) in dioxane (15 mL) and H₂O (0.5 mL) was added XPHOS-PD-G2 (160 mg, 204 umol) and K₃PO₄ (1.30 g, 6.12 mmol). The reaction mixture was stirred at 80° C. for 12 hr. On completion, the reaction mixture was filtered and concentrated in vacuo. The residue was purified by reverse phase (0.1% FA condition) to give the title compound (0.50 g, 35% yield) as white solid. ¹H NMR (400 MHz, DMSO-d₆) δ 10.95 (s, 1H), 6.90-6.84 (m, 1H), 6.84-6.77 (m, 1H), 5.78 (s, 1H), 4.01 (s, 2H), 3.63-3.48 (m, 2H), 3.23 (s, 3H), 2.41-2.22 (m, 2H), 1.43 (s, 9H); LC-MS (ESI⁺) m/z 348.0 (M+H)⁺.

### Step 2—Tert-butyl 4-(5-fluoro-3-methyl-2-oxo-1H-benzimidazol-4-yl)piperidine-1-carboxylate

To a mixture of tert-butyl 4-(5-fluoro-3-methyl-2-oxo-1H-benzimidazol-4-yl)-3,6-dihydro-2H-pyridine-1-carboxylate (500 mg, 1.44 mmol) in MeOH (200 mL) was added formic acid (1.22 g, 26.5 mmol), Pd/C (400 mg, 10 wt %) and Pd(OH)₂/C (400 mg, 10 wt %). The reaction mixture was stirred at 60° C. for 48 hours under H₂ (50 Psi) atmosphere. On completion, the reaction mixture was filtered and concentrated in vacuo to give the title compound (350 mg, 69.% yield) as light yellow solid. LC-MS (ESI⁺) m/z 293.9 (M−56+H)⁺.

### Step 3—Tert-butyl 4-[5-fluoro-1-[1-[(4-methoxyphenyl)methyl]-2,6-dioxo-3-piperidyl]-3-methyl-2-oxo-benzimidazol-4-yl]piperidine-1-carboxylate

To a mixture of tert-butyl 4-(5-fluoro-3-methyl-2-oxo-1H-benzimidazol-4-yl)piperidine-1-carboxylate (350 mg, 1.00 mmol) and t-BuOK (168 mg, 1.50 mmol) in THF (8 mL) was added a solution of [1-[(4-methoxyphenyl)methyl]-2,6-dioxo-3-piperidyl]trifluoromethanesulfonate (572 mg, 1.50 mmol, Intermediate IQ) in THF (8 mL) dropwise. The reaction mixture was stirred at 0° C. for 3 hr. On completion, the reaction mixture was quenched with NH₃Cl (2 mL) and concentrated in vacuo. The residue was purified by reverse phase (0.1% FA condition) to give the title compound (350 mg, 60% yield) as Brown solid. LC-MS (ESI⁺) m/z 525.1 (M−56+H)⁺.

### Step 4—3-[5-Fluoro-3-methyl-2-oxo-4-(4-piperidyl)benzimidazol-1-yl]piperidine-2,6-dione

To a mixture of tert-butyl 4-[5-fluoro-1-[1-[(4-methoxyphenyl)methyl]-2,6-dioxo-3-piperidyl]-3-methyl-2-oxo-benzimidazol-4-yl]piperidine-1-carboxylate (260 mg, 447 umol) in TFA (4 mL) was added TfOH (0.1 mL). The reaction mixture was stirred at 70° C. for 1 hour. On completion, the reaction mixture was concentrated in vacuo to give the title compound (210 mg, 98% yield, TFA) as red oil. LC-MS (ESI⁺) m/z 361.1 (M+H)⁺.

### N-[2-(4-formylcyclohexyl)-6-(trifluoromethyl)indazol-5-yl]-6-(trifluoromethyl) pyridine-2-carboxamide (Intermediate BSQ)

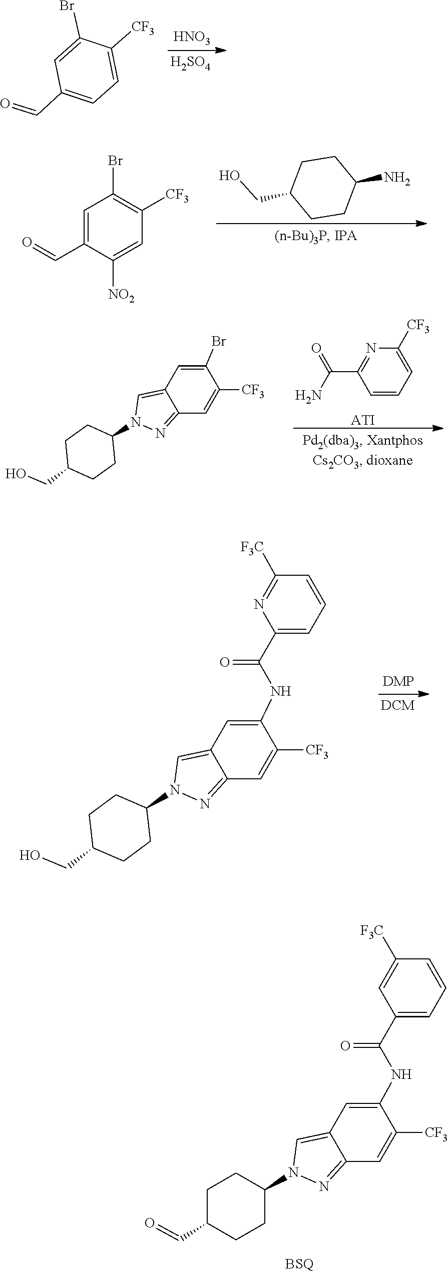

### Step 1—5-Bromo-2-nitro-4-(trifluoromethyl)benzaldehyde

To a mixture of 3-bromo-4-(trifluoromethyl) benzaldehyde (4.50 g, 17.7 mmol, CAS #891180-59-9) in H₂SO₄ (50 mL) was added HNO₃ (1.46 g, 23.1 mmol) at 0° C. Then the reaction mixture was stirred at 25° C. for 2 hours. On completion, the reaction mixture was diluted with water (100 mL), light yellow solid was formed and filtered to give the title compound (5.30 g, 99% yield) as light yellow solid. ¹H NMR (400 MHz, DMSO-d₆) δ 10.26 (s, 1H), 8.49 (s, 1H), 8.31 (s, 1H).

### Step 2—[4-[5-Bromo-6-(trifluoromethyl) indazol-2-yl]cyclohexyl]methanol

To a mixture of 5-bromo-2-nitro-4-(trifluoromethyl) benzaldehyde (4.80 g, 16.1 mmol) and (4-aminocyclohexyl) methanol (2.29 g, 17.7 mmol, CAS #1467-84-1) in IPA (55 mL) and the reaction mixture was stirred at 80° C. for 12 hours. Then to the reaction mixture was added tributylphosphane (9.78 g, 48.3 mmol) at 25° C., and the reaction mixture was stirred at 80° C. for 4 hours. On completion, the reaction mixture was concentrated in vacuo. The residue was purified by column chromatography to give the title compound (3.40 g, 56% yield) as white solid. ¹H NMR (400 MHz, DMSO-d₆) δ 8.56 (s, 1H), 8.26 (s, 1H), 8.21 (s, 1H), 4.58-4.52 (m, 1H), 4.50 (t, J=5.6 Hz, 1H), 3.28 (t, J=5.6 Hz, 2H), 2.17-2.11 (m, 2H), 1.93-1.86 (m, 4H), 1.53-1.43 (m, 1H), 1.20-1.10 (m, 2H).

### Step 3—N-[2-[4-(hydroxymethyl)cyclohexyl]-6-(trifluoromethyl) indazol-5-yl]-6-(trifluoromethyl) pyridine-2-carboxamide

To a mixture of [4-[5-bromo-6-(trifluoromethyl)indazol-2-yl]cyclohexyl]methanol (200 mg, 530 umol) and 6-(trifluoromethyl)pyridine-2-carboxamide (110 mg, 583 umol, Intermediate ATI) in dioxane (5 mL) was added Pd₂(dba)₃ (48.5 mg, 53.0 umol), Xantphos (30.6 mg, 53.0 umol) and Cs₂CO₃ (345 mg, 1.06 mmol) and the reaction mixture was stirred at 80° C. for 12 hours. On completion, the residue was diluted with water (10 mL), then the residue was extracted with EA (3×50 mL). The combined organic layer was dried over Na₂SO₄, filtered and concentrated in vacuo to give a residue. The residue was purified by reverse phase (0.1% FA condition) to give the title compound (130 mg, 50% yield) as white solid. LC-MS (ESI⁺) m/z 487.1 (M+H)⁺.

### Step 4—N-[2-(4-formylcyclohexyl)-6-(trifluoromethyl)indazol-5-yl]-6-(trifluoromethyl) pyridine-2-carboxamide

To a mixture of N-[2-[4-(hydroxymethyl)cyclohexyl]-6-(trifluoromethyl)indazol-5-yl]-6-(trifluo-romethyl)pyridine-2-carboxamide (120 mg, 246 umol) in DCM (2 mL) was added DMP (156 mg, 370 umol) and the reaction mixture was stirred at 25° C. for 3 hours. On completion, the residue was diluted with water (50 mL), then the residue was extracted with DCM (3×70 mL). The combined organic layer was dried over Na₂SO₄, filtered and concentrated in vacuo to give the title compound (120 mg, 95% yield) as light yellow solid. LC-MS (ESI⁺) m/z 485.1 (M+H)⁺.

### 3-[4-[1-[[4-(5-Amino-6-methoxy-indazol-2-yl)cyclohexyl]methyl]-4-piperidyl]-3-methyl-2-oxo-benzimidazol-1-yl]piperidine-2,6-dione (Intermediate BSS)

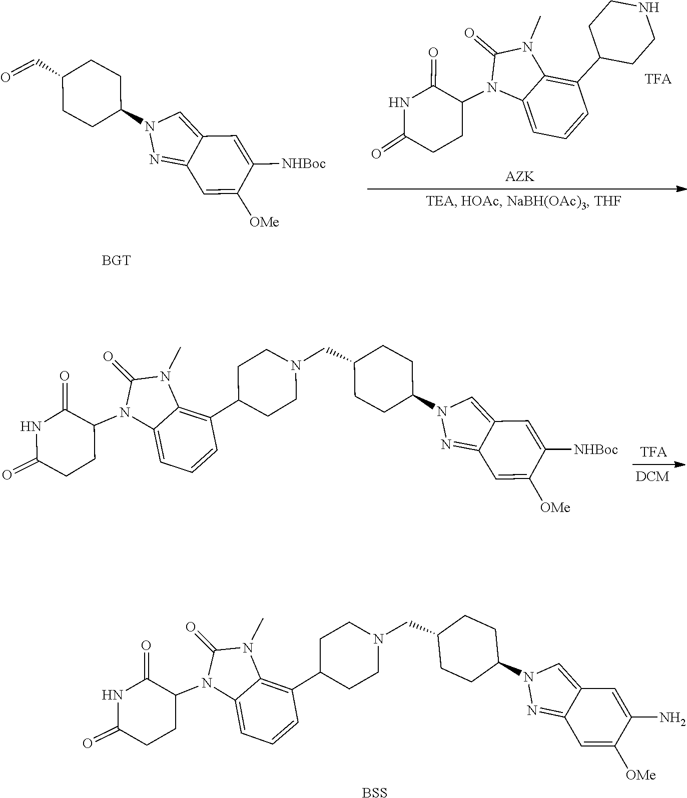 [SMILES: COc1cc2nn([C@H]3CC[C@H](C=O)CC3)cc2cc1NC(=O)OC(C)(C)C ; Cn1c(=O)n(C2CCC(=O)NC2=O)c2cccc(C3CCNCC3)c21 ; COc1cc2nn([C@H]3CC[C@H](CN4CCC(c5cccc6c5n(C)c(=O)n6C5CCC(=O)NC5=O)CC4)CC3)cc2cc1NC(=O)OC(C)(C)C ; COc1cc2nn([C@H]3CC[C@H](CN4CCC(c5cccc6c5n(C)c(=O)n6C5CCC(=O)NC5=O)CC4)CC3)cc2cc1N ; "BSS"]

### Step 1—Tert-butyl N-[2-[4-[[4-[1-(2,6-dioxo-3-piperidyl)-3-methyl-2-oxo-benzimidazol-4-yl]-1-piperidyl]methyl]cyclohexyl]-6-methoxy-indazol-5-yl]carbamate

To a solution of 3-[3-methyl-2-oxo-4-(4-piperidyl)benzimidazol-1-yl]piperidine-2,6-dione (513 mg, 1.12 mmol, TFA, Intermediate AZK) in THF (8 mL) was added TEA (313.08 uL, 2.25 mmol) at −10° C. Then HOAc (128 uL, 2.25 mmol) and tert-butyl N-[2-(4-formylcyclohexyl)-6-methoxy-indazol-5-yl]carbamate (420 mg, 1.12 mmol, Intermediate BGT) was added to the mixture at −10° C. The mixture was stirred at −10° C. for 0.5 hr, then NaBH(OAc)₃ (357 mg, 1.69 mmol) was added at −10° C. and the mixture was stirred at −10° C. for 1 hr. On completion, the mixture was quenched by water (0.2 mL), and then concentrated in vacuo to give a residue. The residue was purified by reverse-phase (FA condition) to give the title compound (230 mg, 29% yield) as a white solid. ¹H NMR (400 MHz, DMSO-d₆) δ 11.09 (s, 1H), 8.17 (d, J=7.2 Hz, 1H), 7.89 (s, 1H), 7.77 (s, 1H), 7.04-6.95 (m, 4H), 5.37 (dd, J=5.2, 12.4 Hz, 1H), 4.40-4.27 (m, 1H), 3.85 (s, 3H), 3.59 (s, 3H), 3.01 (d, J=10.4 Hz, 2H), 2.97-2.80 (m, 2H), 2.77-2.69 (m, 1H), 2.62 (d, J=17.6 Hz, 1H), 2.23 (d, J=6.8 Hz, 2H), 2.17-2.07 (m, 4H), 1.98-1.95 (m, 3H), 1.92-1.85 (m, 2H), 1.80 (s, 4H), 1.72-1.60 (m, 1H), 1.46 (s, 9H), 1.12 (q, J=12.4 Hz, 2H); LC-MS (ESI⁺) m/z 700.2 (M+H)⁺.

### Step 2—3-[4-[1-[[4-(5-Amino-6-methoxy-indazol-2-yl)cyclohexyl]methyl]-4-piperidyl]-3-methyl-2-oxo-benzimidazol-1-yl]piperidine-2,6-dione

To a solution of tert-butyl N-[2-[4-[[4-[1-(2,6-dioxo-3-piperidyl)-3-methyl-2-oxo-benzimidazol-4-yl]-1-piperidyl]methyl]cyclohexyl]-6-methoxy-indazol-5-yl]carbamate (70.0 mg, 100 umol) in DCM (1 mL) was added TFA (74.0 uL, 1.00 mmol). The mixture was stirred at 25° C. for 1 hr. On completion, the mixture was concentrated in vacuo to give the title compound (70.0 mg, 90% yield, TFA) as an off-white oil. LC-MS (ESI⁺) m/z 600.2 (M+H)⁺.

### 6-(5-cyanopyrrolo[2,3-b]pyridin-1-yl)-N-[(4-formylcyclohexyl)methyl]-4-(isopropylamino) pyridine-3-carboxamide (Intermediate BSU)

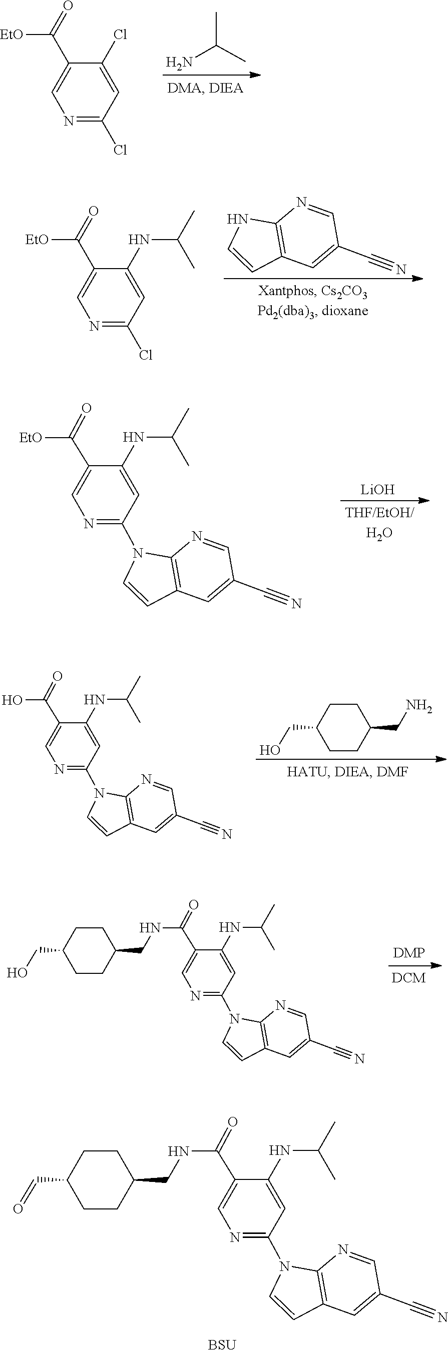

### Step 1—Ethyl 6-chloro-4-(isopropylamino)pyridine-3-carboxylate

To a solution of ethyl 4,6-dichloropyridine-3-carboxylate (1 g, 4.54 mmol, CAS #40296-46-6) in DMA (10 mL) was added DIEA (2.94 g, 22.7 mmol, 3.96 mL) and propan-2-amine (537 mg, 9.09 mmol, CAS #4432-77-3). The reaction mixture was stirred at 50° C. for 3 hrs. On completion, the reaction mixture was diluted with EtOAc (50 mL) and washed with water (50 mL×3). The organic layer was dried over Na₂SO₄, filtered and concentrated in vacuo. The residue was purified by column chromatography (SiO₂, petroleum ether/ethyl acetate=10/1, P1:R_{f}=0.5) to give title compound (0.968 g, 87% yield) as yellow solid. ¹H NMR (400 MHz, DMSO-d₆) δ 8.53 (s, 1H), 7.99 (d, J=6.8 Hz, 1H), 6.83 (s, 1H), 4.29 (q, J=7.2 Hz, 2H), 3.92-3.79 (m, 1H), 1.31 (t, J=7.2 Hz, 3H), 1.20 (d, J=6.4 Hz, 6H).

### Step 2—Ethyl 6-(5-cyanopyrrolo[2,3-b]pyridin-1-yl)-4-(isopropylamino)pyridine-3-carboxylate

To a solution of ethyl 6-chloro-4-(isopropylamino)pyridine-3-carboxylate (868 mg, 3.58 mmol) and 1H-pyrrolo[2,3-b]pyridine-5-carbonitrile (511 mg, 3.58 mmol, CAS #517918-95-5) in dioxane (9 mL) was added Xantphos (206 mg, 357 umol) and Cs₂CO₃ (2.33 g, 7.15 mmol). The reaction mixture was purged with N₂ several times, then Pd₂(dba)₃ (327 mg, 357 umol) was added and the mixture was purged with N₂ again. The mixture was then stirred at 110° C. for 16 hrs under N₂. On completion, the reaction mixture was filtered and concentrated in vacuo. The residue was purified by column chromatography (SiO₂, petroleum ether/ethyl acetate=10/1 to 5/1, P1:R_{f}=0.5) to give title compound (500 mg, 40% yield) as yellow solid. ¹H NMR (400 MHz, CDCl₃) δ 8.83 (s, 1H), 8.67 (s, 1H), 8.57 (d, J=4.0 Hz, 1H), 8.25 (d, J=12.0 Hz, 2H), 8.19 (s, 1H), 6.71 (d, J=3.6 Hz, 1H), 4.37 (q, J=7.2 Hz, 2H), 3.98-3.88 (m, 1H), 1.44 (s, 9H).

### Step 3—6-(5-cyanopyrrolo[2,3-b]pyridin-1-yl)-4-(isopropylamino)pyridine-3-carboxylic acid

To a solution of ethyl 6-(5-cyanopyrrolo[2,3-b]pyridin-1-yl)-4-(isopropylamino)pyridine-3-carboxylate (400 mg, 1.14 mmol) in EtOH (1 mL), THF (4 mL) and H₂O (0.6 mL) was added LiOH H₂O (480 mg, 11.4 mmol). The mixture was then stirred at 50° C. for 9 hrs. On completion, the reaction mixture was filtered and the filtrate was diluted with water (4 mL). The aqueous layer was acidified to pH 5-6 using 2 M HCl and freeze-drying. The product was dissolved in DCM:MeOH=10:1 (11 mL) and filtered. The filtrate was concentrated in vacuo to give title compound (220 mg, 59% yield) as yellow solid. ¹H NMR (400 MHz, DMSO-d₆) δ 10.02 (s, 1H), 8.77 (d, J=2.0 Hz, 1H), 8.66-8.61 (m, 2H), 8.51 (d, J=4.0 Hz, 1H), 7.83 (s, 1H), 6.82 (d, J=4.0 Hz, 1H), 3.75-3.65 (m, 1H), 1.33 (d, J=6.6 Hz, 1H), 1.27 (d, J=6.4 Hz, 6H).

### Step 4—6-(5-cyanopyrrolo[2,3-b]pyridin-1-yl)-N-[[4-(hydroxymethyl)cyclohexyl]methyl]-4-(isopropylamino)pyridine-3-carboxamide

To a solution of 6-(5-cyanopyrrolo[2,3-b]pyridin-1-yl)-4-(isopropylamino)pyridine-3-carboxylic acid (200 mg, 622 umol) and [4-(aminomethyl)cyclohexyl]methanol (115 mg, 809 umol, CAS #17879-23-1) in DMF (4 mL) were added DIEA (160 mg, 1.24 mmol) and HATU (473 mg, 1.24 mmol) at 25° C. The reaction mixture was stirred at 25° C. for 1 hr. On completion, the reaction mixture was diluted with EtOAc (20 ml). The organic layer was washed with water (20 mL×3) and concentrated in vacuo. The residue was purified by column chromatography (SiO₂, petroleum ether/ethyl acetate=1/1, P1:R_{f}=0.3) to give title compound (260 mg, 93% yield) as pink solid. ¹H NMR (400 MHz, DMSO-d₆) δ 8.80 (d, J=2.0 Hz, 1H), 8.66 (d, J=2.0 Hz, 1H), 8.62 (d, J=7.2 Hz, 1H), 8.57 (s, 1H), 8.56-8.53 (m, 1H), 8.52 (d, J=4.0 Hz, 1H), 8.07 (s, 1H), 6.88 (d, J=4.0 Hz, 1H), 4.34 (t, J=5.2 Hz, 1H), 3.83-3.70 (m, 1H), 3.20 (t, J=5.6 Hz, 2H), 3.09 (t, J=6.4 Hz, 2H), 2.69 (s, 3H), 1.81-1.71 (m, 4H), 1.55-1.44 (m, 1H), 1.29 (d, J=6.4 Hz, 6H).

### Step 5—6-(5-cyanopyrrolo[2,3-b]pyridin-1-yl)-N-[(4-formylcyclohexyl)methyl]-4-(isopropylamino) pyridine-3-carboxamide

To a solution of 6-(5-cyanopyrrolo[2,3-b]pyridin-1-yl)-N-[[4-(hydroxymethyl)cyclohexyl]methyl]-4-(isopropylamino)pyridine-3-carboxamide (210 mg, 470 umol) in DCM (4 mL) and DMF (0.1 mL) was added DMP (398 mg, 940 umol). The reaction was stirred at 25° C. for 1 hr. On completion, the reaction mixture was quenched with Na₂S₂O₃ (6 mL) and NaHCO₃ (6 mL) aqueous and the mixture was extracted with DCM (30 mL×2). The combined organic layers were concentrated in vacuo to give title compound (155 mg, 348 umol, 74% yield) as yellow solid. LC-MS (ESI⁺) m/z 445.2 (M+H)⁺.

### 3-[5-Chloro-3-methyl-2-oxo-4-(4-piperidyl)benzimidazol-1-yl]piperidine-2,6-dione (Intermediate BSV)

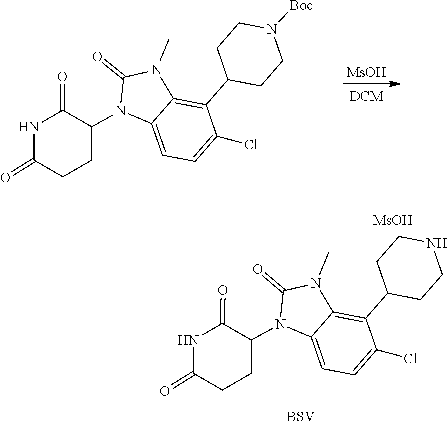

To a mixture of tert-butyl 4-[5-chloro-1-(2, 6-dioxo-3-piperidyl)-3-methyl-2-oxo-benzimidazol-4-yl]piperidine-1-carboxylate (50.0 mg, 104 umol, synthesized via Step 1 of Intermediate BQL) in DCM (2 mL) was added MsOH (30.2 mg, 314 umol). The reaction mixture was stirred at 60° C. for 1 hour. On completion, the reaction mixture was filtered and the residue was concentrated in vacuo to give the title compound (45.0 mg) as white solid. LC-MS (ESI⁺) m/z 377.1 (M+H)⁺.

### N-[2-[3-(iodomethyl)cyclobutyl]-6-methoxy-indazol-5-yl]-6-(trifluoromethyl)pyridine-2-carboxamide (Intermediate BSW)

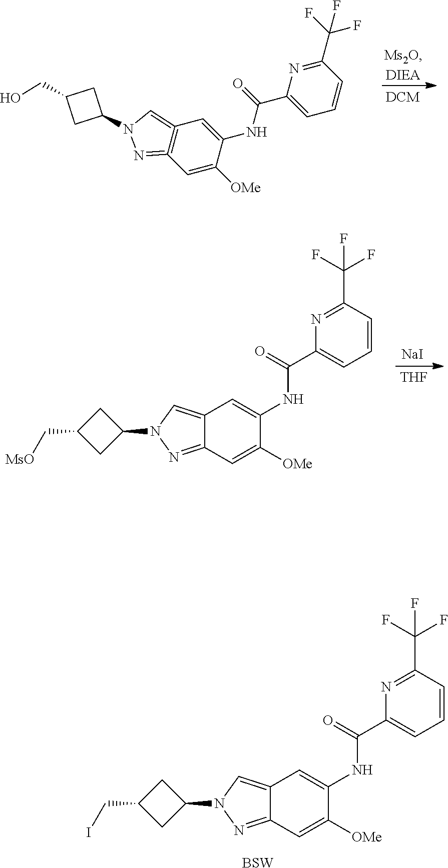

### Step 1—3-[6-Methoxy-5-[[6-(trifluoromethyl)pyridine-2-carbonyl]amino]indazol-2-yl]cyclobutyl]methyl methanesulfonate

To a mixture of N-[2-[3-(hydroxymethyl)cyclobutyl]-6-methoxy-indazol-5-yl]-6-(trifluoromethyl) pyridine-2-carboxamide (230 mg, 547 umol, synthesized via Steps 1-3 of Intermediate BQI) and DIEA (212 mg, 1.64 mmol, 285 uL) in THF (5 mL) was added methylsulfonyl methanesulfonate (142 mg, 820 umol). The reaction mixture was stirred at 25° C. for 12 hr. On completion, the reaction mixture was diluted with water (10 mL) and extracted with EA(2×20 mL). The combined organic layer was dried over Na₂SO₄, filtered and concentrated in vacuo to give a residue. The residue was purified by column chromatography to give the title compound (210 mg, 77% yield) as yellow solid. LC-MS (ESI⁺) m/z 499.1 (M+H)⁺.

### Step 2—N-[2-[3-(iodomethyl)cyclobutyl]-6-methoxy-indazol-5-yl]-6-(trifluoromethyl)pyridine-2-carboxamide

To a mixture of [3-[6-methoxy-5-[[6-(trifluoromethyl)pyridine-2-carbonyl]amino]indazol-2-yl]cyclobutyl]methyl methanesulfonate (210 mg, 421 umol) in THF (8 mL) was added NaI (284 mg, 1.90 mmol). The reaction mixture was stirred at 65° C. for 12 hr. On completion, the reaction mixture was filtered and concentrated in vacuo. The residue was diluted with water (10 mL) and extracted with EA(2×30 mL). The combined organic layers was dried over Na₂SO₄, filtered and concentrated in vacuo to give the title compound (210 mg, 396.02 umol, 94.00% yield) as yellow solid. LC-MS (ESI⁺) m/z 531.0 (M+H)⁺.

### N-[6-chloro-2-[3-(iodomethyl)cyclobutyl]indazol-5-yl]-6-(trifluoromethyl)pyridine-2-carboxamide (Intermediate BSX)

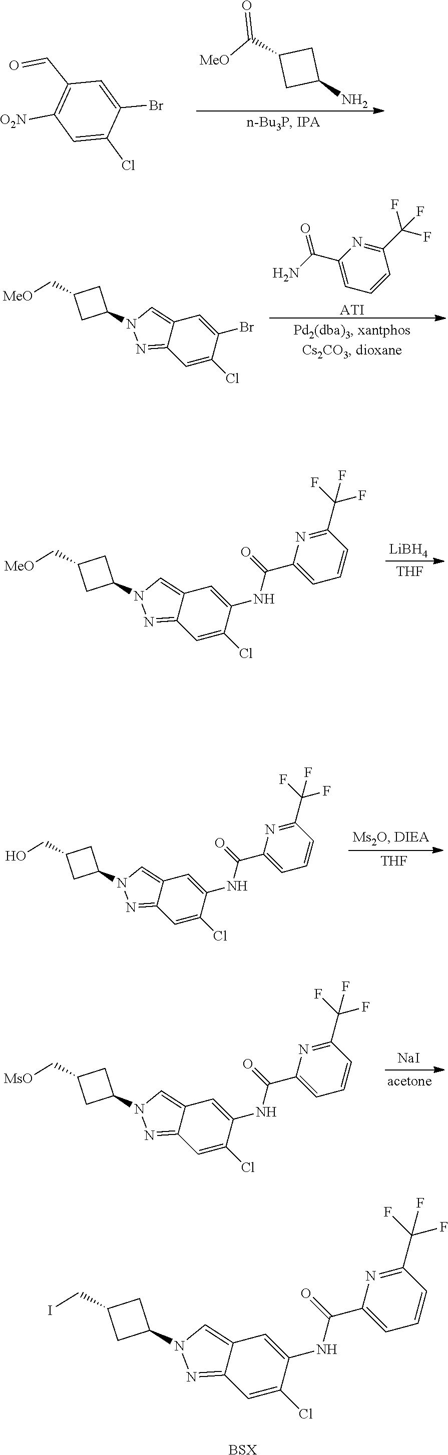

### Step 1—methyl 3-(5-bromo-6-chloro-indazol-2-yl)cyclobutanecarboxylate

To a solution of methyl 3-aminocyclobutanecarboxylate (293 mg, 2.27 mmol, CAS #74316-29-3) and 5-bromo-4-chloro-2-nitro-benzaldehyde (500 mg, 1.89 mmol, synthesized via Step 1 of Intermediate BPQ) in IPA (10 mL) was stirred under N₂ at 20° C. for 3 hrs. Then, tributylphosphane (1.15 g, 5.67 mmol) was added into the mixture and stirred at 80° C. for 4 hr under N₂. On completion, the reaction mixture was concentrated in vacuo. The residue was purified by column chromatography (SiO₂, PE:EA=10:1 to PE:EA=5:1, PE:EA=3:1, P1:Rf=0.4) to give the title compound (220 mg, 33% yield) as yellow solid. ¹H NMR (400 MHz, CDCl₃) δ 7.96 (s, 1H), 7.89 (d, J=6.4 Hz, 2H), 5.35-5.25 (m, 1H), 3.78 (s, 3H), 3.38-3.28 (m, 1H), 3.13-3.03 (m, 2H), 2.91-2.83 (m, 2H).

### Step 2—methyl 3-[6-chloro-5-[[6-(trifluoromethyl)pyridine-2-carbonyl]amino]indazol-2-yl]cyclobutanecarboxylate

A mixture of methyl 3-(5-bromo-6-chloro-indazol-2-yl)cyclobutanecarboxylate (200 mg, 582 umol), 6-(trifluoromethyl)pyridine-2-carboxamide (166 mg, 873 umol, Intermediate ATI), Pd₂(dba)₃ (53.3 mg, 58.2 umol), Xantphos (33.6 mg, 58.2 umol) and Cs₂CO₃ (379 mg, 1.16 mmol) in dioxane (3 mL) was degassed and purged with N₂ 3 times. Then the mixture was stirred at 100° C. for 16 hrs under N₂ atmosphere. On completion, the reaction mixture was diluted with EtOAc (40 mL), filtered and the filter cake was washed with 50% petroleum ether in EtOAc two times and water to give the title compound (190 mg, 72.% yield) as yellow solid. ¹H NMR (400 MHz, DMSO-d₆) δ10.53 (s, 1H), 8.61 (d, J=13.2 Hz, 2H), 8.48-8.44 (m, 1H), 8.44-8.39 (m, 1H), 8.24 (dd, J=0.8, 7.6 Hz, 1H), 7.98 (s, 1H), 5.40-5.30 (m, 1H), 3.70 (s, 3H), 3.37-3.33 (m, 1H), 2.99-2.87 (m, 2H), 2.82-2.73 (m, 2H).

### Step 3—N-(6-chloro-2-((1r, 3r)-3-(hydroxymethyl)cyclobutyl)-2H-indazol-5-yl)-6-(trifluoromethyl) picolinamide

To a solution of methyl 3-[6-chloro-5-[[6-(trifluoromethyl) pyridine-2-carbonyl]amino]indazol-2-yl]cyclobutanecarboxylate (150 mg, 331 umol) in THF (2 mL) was added LiBH₄ (37.5 mg, 1.72 mmol) at 0° C. under N₂. The reaction mixture was then stirred at 20° C. for 3 hrs. On completion, the reaction mixture was quenched with water (0.1 mL) and the reaction residue was diluted with water (20 mL), then extracted with EA (3×50 mL). The combined organic layers ware dried over Na₂SO₄, filtered and the filtrate was concentrated in vacuo. The residue was purified by prep-TLC (SiO₂, DCM:MeOH=20:1, P1:Rf=0.3) to give the title compound (40.0 mg, 28% yield) as white solid. LC-MS (ESI⁺) m/z 425.0 (M+H)⁺.

### Step 4—[3-[6-chloro-5-[[6-(trifluoromethyl)pyridine-2-carbonyl]amino]indazol-2-yl]cyclobutyl]methyl methanesulfonate

To a solution of N-[6-chloro-2-[3-(hydroxymethyl)cyclobutyl]indazol-5-yl]-6-(trifluoromethyl) pyridine-2-carboxamide (40.0 mg, 94.1 umol) in THF (1 mL) was added DIEA (36.5 mg, 282 umol), and the mixture was stirred at 0° C. for 5 mins. Next to the reaction mixture was added methylsulfonyl methanesulfonate (24.6 mg, 141 umol) at 0° C., then the reaction mixture was stirred at 25° C. for 16 hrs. On completion, the reaction mixture was diluted with H₂O (10 mL), and the mixture was extracted with EtOAc (2×20 mL). The combined organic layers ware washed with H₂O (2×10 mL) and dried over Na₂SO₄, filtered and the filtrate was concentrated in vacuo to give the title compound (47.0 mg, 99% yield) as yellow solid. LC-MS (ESI⁺) m/z 503.0 (M+H)⁺.

### Step 5—N-[6-chloro-2-[3-(iodomethyl)cyclobutyl]indazol-5-yl]-6-(trifluoromethyl)pyridine-2-carboxamide

To a solution of [3-[6-chloro-5-[[6-(trifluoromethyl) pyridine-2-carbonyl]amino]indazol-2-yl]cyclobutyl]methyl methanesulfonate (47.0 mg, 93.4 umol) in acetone (2 mL) was added NaI (70.0 mg, 467.30 umol). The reaction mixture was stirred at 60° C. for 16 hrs. On completion, the reaction mixture was diluted with EtOAc (20 mL) and the layers were separated. The organic layer was washed with H₂O (2×10 mL) and dried over Na₂SO₄. The residue was filtered and the filtrate was concentrated in vacuo to give the title compound (49 mg, 98% yield) as yellow solid. ¹H NMR (400 MHz, DMSO-d₆) δ 10.53 (s, 1H), 8.63 (s, 1H), 8.57 (d, J=0.8 Hz, 1H), 8.49-8.44 (m, 1H), 8.44-8.38 (m, 1H), 8.24 (dd, J=1.2, 7.6 Hz, 1H), 7.98 (s, 1H), 5.36-5.27 (m, 1H), 3.60 (d, J=8.0 Hz, 2H), 3.30 (s, 1H), 2.95-2.83 (m, 1H), 2.77-2.68 (m, 2H), 2.39-2.35 (m, 1H).

### 3-[3-methyl-2-oxo-4-(4-piperidyl)benzimidazol-1-yl]piperidine-2,6-dione (Intermediate BSY)

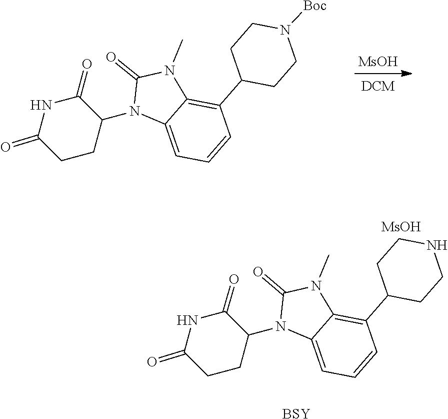

To a solution of tert-butyl 4-[1-(2,6-dioxo-3-piperidyl)-3-methyl-2-oxo-benzimidazol-4-yl]piperidine-1-carboxylate (150 mg, 338 umol, synthesized via Steps 1-2 of Intermediate AZK) in DCM (1 mL) was added methanesulfonic acid (97.7 mg, 1.02 mmol), then the reaction mixture was stirred at 20° C. for 3 hrs. On completion, the reaction mixture was diluted with MTBE (10 mL), and the mixture was stirred at 20° C. for 10 mins. The mixture was filtered and the filter cake was dried in vacuo to give the title compound (100 mg, 86% yield) as brown solid. LC-MS (ESI⁺) m/z 343.1 (M+H)⁺.

### 1-[8-(4-Piperidyl)-4-isoquinolyl]hexahydropyrimidine-2,4-dione (Intermediate BSZ)

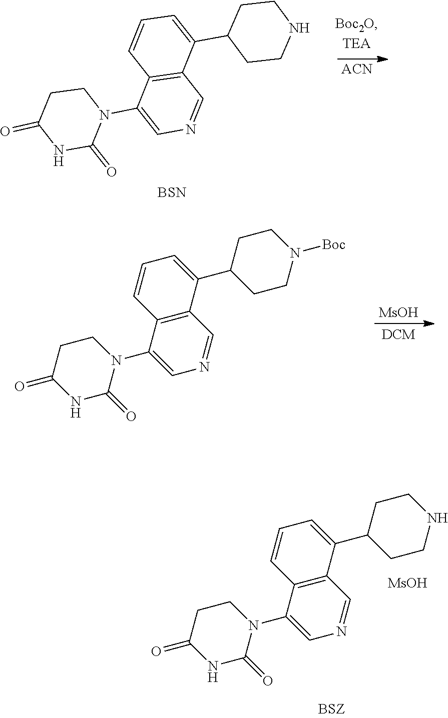

### Step 1—Tert-butyl 4-[4-(2,4-dioxohexahydropyrimidin-1-yl)-8-isoquinolyl]piperidine-1-carboxylate

To a mixture of 1-[8-(4-piperidyl)-4-isoquinolyl]hexahydropyrimidine-2,4-dione (480 mg, 1.09 mmol, TFA, Intermediate BSN) in ACN (10 mL) was added Boc₂O (358 mg, 1.64 mmol) and TEA (332 mg, 3.28 mmol). The reaction mixture was stirred at 25° C. for 12 hours. On completion, the reaction mixture was filtered and concentrated in vacuo to give the title compound (350 mg, 75% yield) as brown oil. ¹H NMR (400 MHz, DMSO-d6) δ 10.54 (s, 1H), 9.67 (s, 1H), 8.57 (s, 1H), 7.88-7.85 (m, 1H), 7.76 (t, J=7.6 Hz, 1H), 7.61 (d, J=7.6 Hz, 1H), 4.14 (d, J=9.6 Hz, 2H), 3.96-3.88 (m, 1H), 3.83 (t, J=11.6 Hz, 1H), 3.74-3.68 (m, 1H), 3.02-2.93 (m, 2H), 2.79-2.72 (m, 1H), 1.94-1.87 (m, 2H), 1.73-1.59 (m, 3H), 1.44 (s, 9H).

### Step 2—1-[8-(4-Piperidyl)-4-isoquinolyl]hexahydropyrimidine-2,4-dione

To a mixture of tert-butyl 4-[4-(2,4-dioxohexahydropyrimidin-1-yl)-8-isoquinolyl]piperidine-1-carboxylate (60.0 mg, 141 umol) in DCM (2 mL) was added MsOH (40.7 mg, 424 umol) and the reaction mixture was stirred at 25° C. for 1 hour. On completion, the reaction mixture was triturated with MTBE (3 mL) and filtered to afford a white solid which was collected and dried in vacuo to give the title compound (45.0 mg, 98% yield) as white solid. LC-MS (ESI⁺) m/z 325.1 (M+H)+

### 4-[6-Methoxy-5-[[6-(trifluoromethyl)pyridine-2-carbonyl]amino]indazol-2-yl]cyclohexane carboxylic acid (Intermediate AXQ)

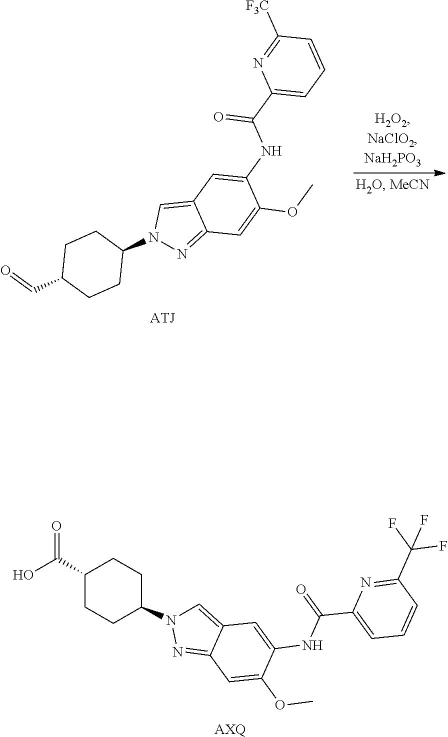

To a solution of N-[2-(4-formylcyclohexyl)-6-methoxy-indazol-5-yl]-6-(trifluoromethyl)pyridine-2-carboxamide (1.00 g, 2.24 mmol, Intermediate ATJ) and NaH₂PO₄ (1.34 g, 11.2 mmol) in ACN (20 mL) was added H₂O₂(507 mg, 4.48 mmol, 30% solution) dropwise at 0° C. Then a solution of sodium chlorite (1.42 g, 15.6 mmol) in H₂O (10 mL) was added and the reaction mixture was stirred at 0-25° C. for 1 hour. On completion, the reaction mixture was diluted with ACN (20 mL), quenched with Na₂SO₃ (10 mL) aqueous solution and filtered. The solid was dried in vacuo to give the title compound (800 mg, 80% yield) as brown solid. LC-MS (ESI⁺) m/z 463.1 (M+H)⁺.

### N-[2-(4-formylcyclohexyl)indazol-5-yl]-6-(trifluoromethyl)pyridine-2-carboxamide (Intermediate BTW)

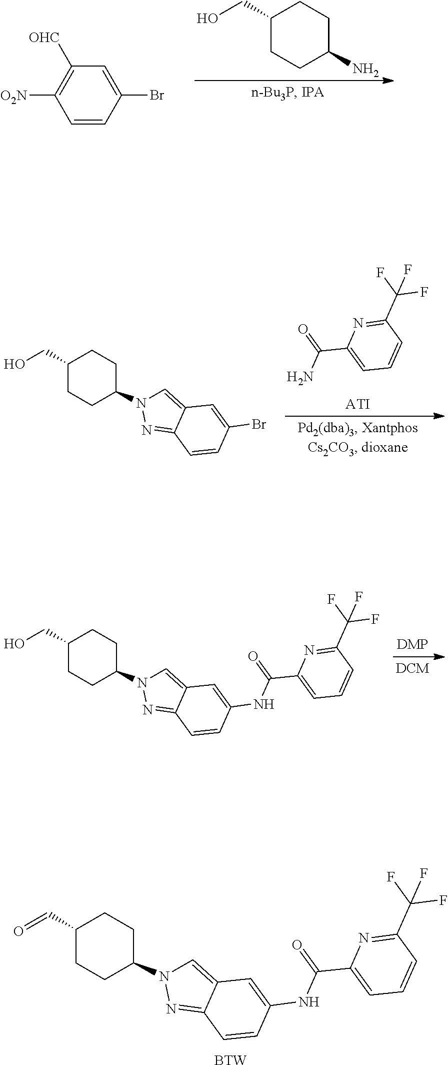

### Step 1—[4-(5-Bromoindazol-2-yl)cyclohexyl]methanol

To a solution of 5-bromo-2-nitro-benzaldehyde (4.00 g, 17.3 mmol, CAS #20357-20-4) and (4-aminocyclohexyl)methanol (2.47 g, 19.1 mmol, CAS #1467-84-1) in IPA (60 mL), then the reaction mixture was stirred at 80° C. for 4 hrs under N₂. Next, the mixture was cooled to 25° C. and tributylphosphane (3.52 g, 17.3 mmol, 4.29 mL) was added. Then the mixture was stirred at 80° C. for 16 hrs under N₂. On completion, the reaction mixture was diluted with EA (50 mL) and extracted with EA (3×100 mL). The combined organic layers were washed with brine (2×100 mL), dried over anhydrous Na₂SO₄, filtered and concentrated to give the title compound (1.8 g, 33% yield) as a white solid. ¹H NMR (400 MHz, DMSO-d₆) δ 8.39 (d, J=0.8 Hz, 1H), 7.93 (d, J=1.6 Hz, 1H), 7.58 (d, J=9.2 Hz, 1H), 7.29 (dd, J=2.0, 8.8 Hz, 1H), 4.54-4.48 (m, 1H), 4.48-4.36 (m, 1H), 3.28 (t, J=5.6 Hz, 2H), 2.18-2.06 (m, 2H), 1.95-1.81 (m, 3H), 1.96-1.79 (m, 1H), 1.47 (m, 1H), 1.22-1.06 (m, 2H); LC-MS (ESI⁺) m/z 308.9 (M+H)⁺.

### Step 2—N-[2-[4-(hydroxymethyl)cyclohexyl]indazol-5-yl]-6-(trifluoromethyl)pyridine-2-carboxamide

To a solution of [4-(5-bromoindazol-2-yl)cyclohexyl]methanol (800 mg, 2.59 mmol) and 6-(trifluoromethyl)pyridine-2-carboxamide (688 mg, 3.62 mmol, Intermediate ATI) in dioxane (15 mL) was added Pd₂(dba)₃ (236 mg, 258 umol), Xantphos (299 mg, 517 umol) and Cs₂CO₃ (1.69 g, 5.17 mmol), then the reaction mixture was stirred at 100° C. for 6 hrs under N₂. On completion, the reaction mixture was filtered and the filtrate was concentrated in vacuo to give a residue. The residue was purified by column chromatography (SiO₂, PE:EA=1:1 to PE:EA=0:1) to give the title compound (800 mg, 73% yield) as a white solid. ¹H NMR (400 MHz, DMSO-d₆) δ 10.35 (s, 1H), 8.44-8.31 (m, 3H), 8.29 (d, 1H), 8.17 (d, J=7.2 Hz, 1H), 7.65-7.58 (m, 1H), 7.58-7.51 (m, 1H), 4.49 (t, J=5.2 Hz, 1H), 4.42 (m, 1H), 3.29 (t, J=5.6 Hz, 2H), 2.15 (d, J=9.6 Hz, 2H), 1.97-1.84 (m, 4H), 1.49 (m, 1H), 1.24-1.08 (m, 2H); LC-MS (ESI⁺) m/z 419.3 (M+H)⁺.

### Step 3—N-[2-(4-formylcyclohexyl)indazol-5-yl]-6-(trifluoromethyl)pyridine-2-carboxamide

To a solution of N-[2-[4-(hydroxymethyl)cyclohexyl]indazol-5-yl]-6-(trifluoromethyl)pyridine-2-carboxamide (400 mg, 956 umol) in DCM (3 mL) was added DMP (608 mg, 1.43 mmol, 443 uL), then the reaction mixture was stirred at 25° C. for 2 hrs. On completion, the reaction mixture was quenched with Na₂S₂O₃ (10 mL) and NaHCO₃ (10 mL) and extracted with DCM (2×40 mL). The combined organic phase was washed with NaHCO₃ (20 mL) and brine (2×20 mL), dried with anhydrous Na₂SO₄, filtered and concentrated in vacuo to give the title compound (390 mg, 97% yield) as a brown solid. LC-MS (ESI⁺) m/z 417.3 (M+H)⁺.

### 3-fluoro-5-(trifluoromethyl)benzoic acid (16.8 mg, 80.7 umol, CAS #161622-05-5) (Intermediate BTX)

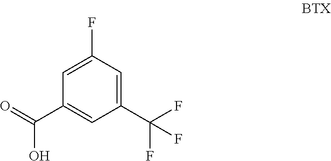 [SMILES: O=C(O)c1cc(F)cc(C(F)(F)F)c1]

### 2-ethynyl-6-(trifluoromethyl)pyridine (Intermediate BTY)

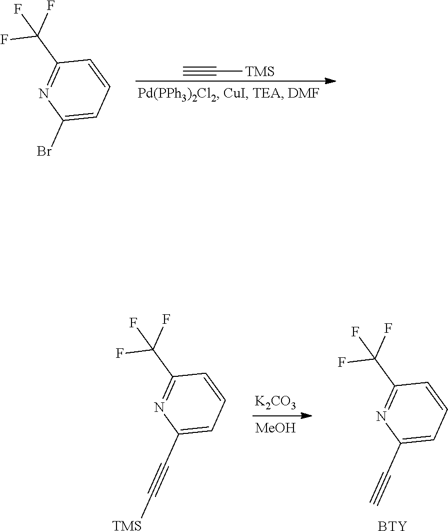

### Step 1—Trimethyl-[2-[6-(trifluoromethyl)-2-pyridyl]ethynyl]silane

To a solution of 2-bromo-6-(trifluoromethyl)pyridine (2 g, 8.85 mmol, CAS #189278-27-1), TEA (2.69 g, 26.5 mmol) and ethynyl(trimethyl)silane (2.17 g, 22.1 mmol, CAS #1066-54-2) in DMF (20 mL) was added Pd(PPh₃)₂Cl₂ (621 mg, 884 umol) and CuI (337 mg, 1.77 mmol) under N₂. The reaction was stirred at 110° C. for 16 hrs. On completion, the reaction mixture was diluted with EtOAc (100 mL), which was then washed with brine (4×50 mL). The organic layer was separated and concentrated in vacuo. The residue was purified by column chromatography (SiO₂, petroleum ether/ethyl acetate=1/0 to 10/1) to give the title compound (1.1 g, 51% yield) as brown solid. ¹H NMR (400 MHz, CDCl₃) δ 7.80-7.71 (m, 1H), 7.54 (t, J=7.6 Hz, 2H), 0.24-0.15 (m, 9H).

### Step 2—2-ethynyl-6-(trifluoromethyl)pyridine

To a solution of trimethyl-[2-[6-(trifluoromethyl)-2-pyridyl]ethynyl]silane (400 mg, 1.64 mmol) in MeOH (4 mL) was added K₂CO₃ (249 mg, 1.81 mmol), and the reaction mixture was stirred at 25° C. for 1 hr. On completion, the reaction mixture was diluted with EtOAc (10 mL) and filtered. Then, the filtrate was concentrated in vacuo and the residue was purified by prep-TLC (PE:EA=10:1, Rf=0.47) to give the title compound (260 mg, 92% yield) as yellow oil. ¹H NMR (400 MHz, CDCl₃) δ=7.91-7.84 (m, 1H), 7.67 (d, J=8.0 Hz, 2H), 3.26 (s, 1H).

### ((1R,4R)-4-(6-methoxy-5-(4,4,5,5-tetramethyl-1,3,2-dioxaborolan-2-yl)-2H-indazol-2-yl) cyclohexyl)methanol (Intermediate BTZ)

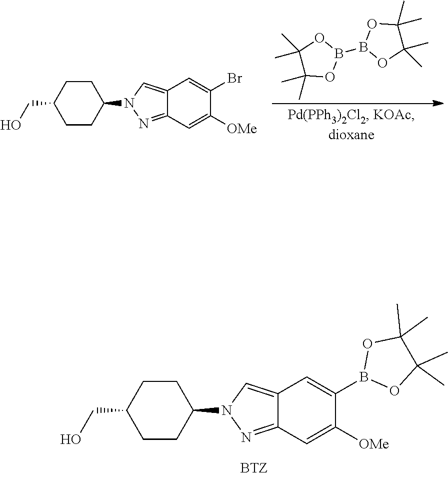

A mixture of [4-(5-bromo-6-methoxy-indazol-2-yl)cyclohexyl]methanol (1.00 g, 2.95 mmol, synthesized via Steps 1-3 of Intermediate ATE), 4,4,5,5-tetramethyl-2-(4,4,5,5-tetramethyl-1,3,2-dioxaborolan-2-yl)-1,3,2-dioxaborolane (973 mg, 3.83 mmol, CAS #73183-34-3), Pd(PPh₃)₂Cl₂ (206 mg, 294 umol) and KOAc (578 mg, 5.90 mmol) in dioxane (15 mL) was degassed and purged with N₂ three times, and the mixture was stirred at 90° C. for 16 hrs under N₂ atmosphere for 16 hrs. On completion, the reaction mixture was diluted with EtOAc (30 mL) and filtered. The filtrate was concentrated in vacuo and the residue was purified by flash silica gel chromatography (Silica Flash Column, Eluent of 20˜50% Ethyl acetate/Petroleum ether gradient) to give the title compound (400 mg, 35% yield) as yellow solid. ¹H NMR (400 MHz, CDCl₃) δ 8.06 (s, 1H), 7.85 (s, 1H), 7.28 (s, 1H), 6.95 (s, 1H), 4.36-4.27 (m, 1H), 3.92-3.84 (m, 3H), 3.57 (d, J=5.6 Hz, 2H), 2.33 (d, J=14.4 Hz, 2H), 2.05-1.90 (m, 3H), 1.72-1.63 (m, 2H), 1.39 (s, 9H), 1.28-1.19 (m, 2H).

### (1R,4R)-4-(6-methoxy-5-(4-(6-(trifluoromethyl)pyridin-2-yl)-1H-1,2,3-triazol-1-yl)-2H-indazol-2-yl)cyclohexanecarbaldehyde (Intermediate BUA)

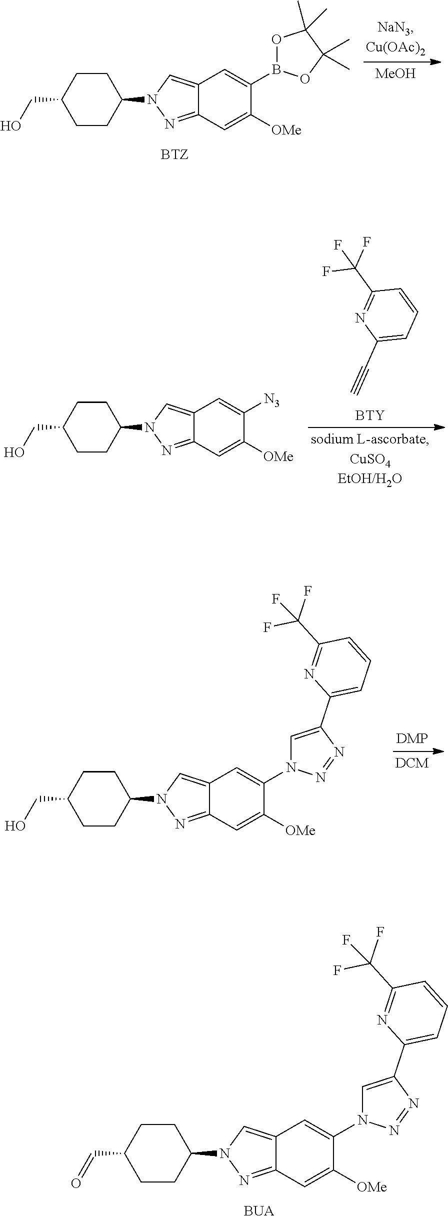

### Step 1—((1R,4R)-4-(5-azido-6-methoxy-2H-indazol-2-yl)cyclohexyl)methanol

To a solution of ((1r, 4r)-4-(6-methoxy-5-(4,4,5,5-tetramethyl-1,3,2-dioxaborolan-2-yl)-2H-indazol-2-yl) cyclohexyl)methanol (370 mg, 957 umol) in MeOH (6 mL) was added NaN₃ (80.9 mg, 1.25 mmol) and Cu(OAc)₂ (173 mg, 957 umol). The reaction mixture was stirred at 50° C. for 3 hrs. On completion, the reaction mixture was diluted with EtOAc (50 mL) and filtered. The filtrate was washed with brine (2×30 mL). The organic layer was separated, dried over Na₂SO₄ and concentrated in vacuo. The residue was purified by prep-TLC (SiO₂, 100% EA, Rf=0.4) to give the title compound (150 mg, 52% yield) as yellow solid. LC-MS (ESI⁺) m/z 302.0 (M+H)⁺.

### Step 2—((1R,4R)-4-(6-methoxy-5-(4-(6-(trifluoromethyl)pyridin-2-yl)-1H-1,2,3-triazol-1-yl)-2H-indazol-2-yl)cyclohexyl)methanol

To a solution of ((1r, 4r)-4-(5-azido-6-methoxy-2H-indazol-2-yl)cyclohexyl)methanol (140 mg, 464 umol) and 2-ethynyl-6-(trifluoromethyl)pyridine (79.5 mg, 464 umol) in EtOH (3 mL) and H₂O (0.5 mL) was added sodium L-ascorbate (27.6 mg, 139 umol) and CuSO₄ (22.2 mg, 139 umol) at 25° C. The reaction was stirred at 25° C. for 16 hrs. On completion, the reaction mixture was diluted with EtOAc (50 mL), which was then washed with brine (30 mL×3). The organic layer was separated, dried over Na₂SO₄ and concentrated in vacuo. The residue was purified by prep-HPLC (column: Phenomenex luna C18 150*25 mm*10 um; mobile phase: [water (0.225% FA)-ACN]; B %: 40%-70%, 11.5 min) to give the title compound (40 mg, 18% yield) as yellow solid. ¹H NMR (400 MHz, CD₃OD) δ 8.83 (s, 1H), 8.43-8.38 (m, 2H), 8.17 (t, J=7.9 Hz, 1H), 8.05 (s, 1H), 7.79 (d, J=7.6 Hz, 1H), 4.51-4.44 (m, 1H), 3.98 (s, 3H), 3.48 (d, J=6.4 Hz, 2H), 2.35-2.26 (m, 2H), 2.11-1.97 (m, 5H), 1.74-1.59 (m, 1H), 1.39-1.21 (m, 2H).

### Step 3—(1R,4R)-4-(6-methoxy-5-(4-(6-(trifluoromethyl)pyridin-2-yl)-1H-1,2,3-triazol-1-yl)-2H-indazol-2-yl)cyclohexanecarbaldehyde

To a solution of ((1r, 4r)-4-(6-methoxy-5-(4-(6-(trifluoromethyl)pyridin-2-yl)-1H-1,2,3-triazol-1-yl)-2H-indazol-2-yl)cyclohexyl)methanol (35.0 mg, 74.1 umol) in DCM (0.5 mL) was added DMP (62.8 mg, 148 umol) at 25° C., then the reaction was stirred at 25° C. for 2 hrs. On completion, the reaction was diluted with DCM (30 mL), then quenched with saturated Na₂S₂O₃ solution (5 mL), followed by addition of saturated NaHCO₃ solution (5 mL). The combined mixture was stirred at 25° C. for 30 min. The organic layer was washed with saturated NaHCO₃ solution (20 mL×3), dried over Na₂SO₄ and concentrated in vacuo to give the title compound (34 mg, 97% yield) as yellow solid. LC-MS (ESI⁺) m/z 471.1 (M+H)⁺.

### 4-bromo-5-methoxy-3-methyl-1H-benzimidazol-2-one (Intermediate BUB)

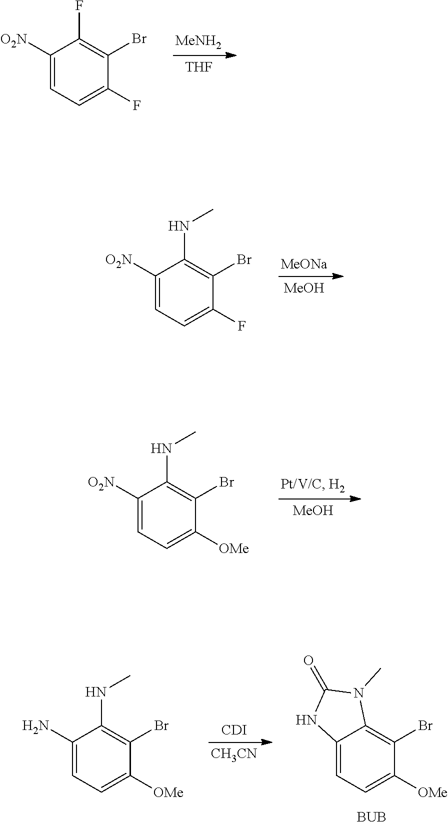

### Step 1—2-bromo-3-fluoro-N-methyl-6-nitro-aniline

To a solution of 2-bromo-1,3-difluoro-4-nitro-benzene (10.0 g, 42.0 mmol, CAS #103977-78-2) in THF (120 mL) was added MeNH₂ (2 M, 42.0 mL) at 0° C. under N₂. The reaction was stirred at 0° C. for 1 hr. On completion, the reaction was diluted with EtOAc (200 mL). The organic layer was washed with water (200 mL×3) and concentrated in vacuo to give title compound (10.0 g, 95% yield) as yellow solid. ¹H NMR (400 MHz, DMSO-d₆) δ 7.88 (dd, J=6.0, 9.2 Hz, 1H), 6.76 (dd, J=7.6, 9.2 Hz, 2H), 2.77 (d, J=5.2 Hz, 3H).

### Step 2—2-bromo-3-methoxy-N-methyl-6-nitro-aniline

To a solution of 2-bromo-3-fluoro-N-methyl-6-nitro-aniline (10.0 g, 40.1 mmol) in MeOH (100 mL) was added NaOMe (2.82 g, 52.2 mmol) at 0° C. Then the reaction was stirred at 25° C. for 16 hrs. On completion, the reaction was concentrated in vacuo. The residue was purified by column chromatography (SiO₂, petroleum ether/ethyl acetate=20/1) to give title compound (10 g, 95% yield) as yellow solid. ¹H NMR (400 MHz, DMSO-d₆) δ 7.90 (d, J=9.6 Hz, 1H), 6.64 (d, J=9.6 Hz, 1H), 6.53 (q, J=4.8 Hz, 1H), 3.92 (s, 3H), 2.77 (d, J=5.6 Hz, 3H).

### Step 3—3-bromo-4-methoxy-N2-methyl-benzene-1,2-diamine

To a solution of 2-bromo-3-methoxy-N-methyl-6-nitro-aniline (5 g, 19.1 mmol) in MeOH (50 mL) was added Pt/V/C (499 mg, 1.92 mmol) under N₂. The suspension was degassed under in vacuo and purged with H₂ several times. The mixture was stirred under H₂ (15 psi) at 25° C. for 16 hours. On completion, the reaction was concentrated in vacuo to give title compound (4.40 g, 99% yield) as red solid. ¹H NMR (400 MHz, CDCl₃) δ 6.64 (d, J=8.4 Hz, 1H), 6.53 (d, J=8.4 Hz, 1H), 3.82 (s, 3H), 3.66 (s, 2H), 2.71 (s, 3H).

### Step 4—4-bromo-5-methoxy-3-methyl-1H-benzimidazol-2-one

A solution of 3-bromo-4-methoxy-N2-methyl-benzene-1,2-diamine (4.4 g, 19.0 mmol) and CDI (3.70 g, 22.8 mmol) in CH₃CN (70 mL) was stirred at 80° C. for 16 hrs under N₂. On completion, the reaction was added into water (200 mL) and filtered. The filter cake was triturated with PE:EA=3:1 at 25° C. for 30 mins and filtered. The solid was collected and dried in vacuo to give title compound (4 g, 81% yield) as gray solid. ¹H NMR (400 MHz, DMSO-d₆) δ=10.93 (s, 1H), 6.90 (d, J=8.4 Hz, 1H), 6.74 (d, J=8.4 Hz, 1H), 3.79 (s, 3H), 3.56 (s, 3H).

### 3-[5-methoxy-3-methyl-2-oxo-4-(4-piperidyl)benzimidazol-1-yl]piperidine-2,6-dione (Intermediate BUC)

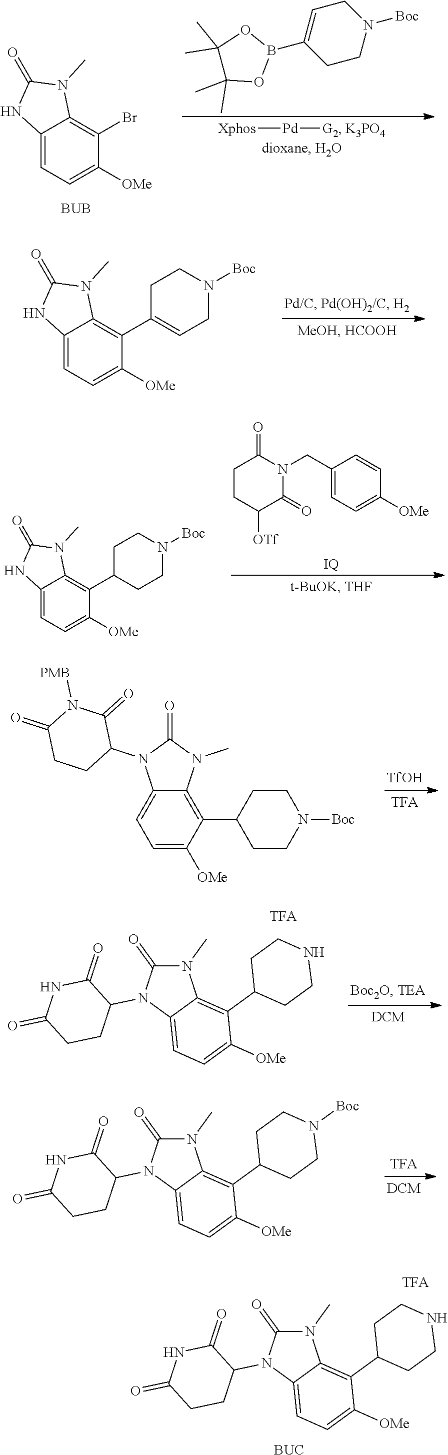

### Step 1—Tert-butyl 4-(5-methoxy-3-methyl-2-oxo-1H-benzimidazol-4-yl)-3,6-dihydro-2H-pyridine-1-carboxylate

A solution of 4-bromo-5-methoxy-3-methyl-1H-benzimidazol-2-one (2.00 g, 7.78 mmol, Intermediate BUB), tert-butyl 4-(4,4,5,5-tetramethyl-1,3,2-dioxaborolan-2-yl)-3,6-dihydro-2H-pyridine-1-carboxylate (3.13 g, 10.1 mmol, CAS #286961-14-6), K₃PO₄ (3.30 g, 15.5 mmol) and XPHOS-PD-G₂ (306 mg, 388 umol) in dioxane (50 mL) and H₂O (10 mL) was stirred at 80° C. for 16 hrs under N₂. On completion, the reaction was filtered and filtrate was concentrated in vacuo. The residue was purified by column chromatography (SiO₂, petroleum ether/ethyl acetate=3/1 to 1/1) to give title compound as gray solid. ¹H NMR (400 MHz, DMSO-d₆) δ 10.67 (s, 1H), 6.81 (d, J=8.4 Hz, 1H), 6.62 (d, J=8.4 Hz, 1H), 5.58 (s, 1H), 3.69 (s, 3H), 3.59-3.47 (m, 2H), 3.18 (s, 3H), 2.42 (s, 1H), 2.14 (d, J=16.8 Hz, 1H), 1.43 (s, 9H), 1.06 (s, 2H).

### Step 2—Tert-butyl 4-(5-methoxy-3-methyl-2-oxo-1H-benzimidazol-4-yl)piperidine-1-carboxylate

To a solution of tert-butyl 4-(5-methoxy-3-methyl-2-oxo-1H-benzimidazol-4-yl)-3,6-dihydro-2H-pyridine-1-carboxylate (600 mg, 1.67 mmol) in MeOH (20 mL) was added HCOOH (80.2 mg, 1.67 mmol), Pd/C (600 mg, 563 umol, 10 wt %) and Pd(OH)₂/C (600 mg, 427 umol, 10 wt %) under N₂ atmosphere. The suspension was degassed under vacuum and purged with H₂ several times. The mixture was stirred under H₂ (50 Psi) at 60° C. for 48 hrs. On completion, the reaction mixture was filtered and filtrate was concentrated in vacuo to give title compound (570 mg, 87% yield) as black solid. ¹H NMR (400 MHz, CDCl₃) δ 6.83 (d, J=8.4 Hz, 1H), 6.55 (d, J=8.4 Hz, 1H), 4.16 (s, 2H), 3.71 (s, 3H), 3.58 (s, 3H), 3.35-3.30 (m, 1H), 2.76-2.62 (m, 2H), 2.43-2.29 (m, 2H), 1.53 (d, J=12.4 Hz, 2H), 1.43 (s, 9H), 1.38-1.36 (m, 1H).

### Step 3—Tert-butyl 4-[5-methoxy-1-[1-[(4-methoxyphenyl)methyl]-2,6-dioxo-3-piperidyl]-3-methyl-2-oxo-benzimidazol-4-yl]piperidine-1-carboxylate

To a solution of tert-butyl 4-(5-methoxy-3-methyl-2-oxo-1H-benzimidazol-4-yl)piperidine-1-carboxylate (1.49 g, 4.12 mmol) in THF (30 mL) was added t-BuOK (693 mg, 6.18 mmol) at 0° C. and the reaction was stirred for 0.5 hr. Then, a solution of [1-[(4-methoxyphenyl) methyl]-2, 6-dioxo-3-piperidyl]trifluoromethanesulfonate (2.36 g, 6.18 mmol, Intermediate IQ) in THF (20 mL) solution was added dropwise into the mixture slowly. The reaction was stirred at 0° C. for 1.5 hrs. On completion, the reaction was quenched with NH₄Cl solution (10 mL). The mixture was diluted with water (150 mL) and extracted with EA (200 mL). The combined layers were washed with water (150 mL×2), dried over Na₂SO₄ and filtered. The filtrate was concentrated in vacuo. The residue was purified by reverse-phase HPLC (0.1% FA condition) and column chromatography (SiO₂, petroleum ether/ethyl acetate=2/1 to 1/2) to give title compound (1.53 g, 62% yield) as yellow solid. ¹H NMR (400 MHz, CDCl₃) δ 7.39-7.35 (m, 2H), 6.83 (d, J=8.4 Hz, 2H), 6.48 (d, J=8.4 Hz, 1H), 6.27 (d, J=8.4 Hz, 1H), 5.22-5.13 (m, 1H), 4.97 (s, 2H), 4.24 (s, 2H), 3.78 (d, J=13.6 Hz, 6H), 3.66 (s, 3H), 3.47-3.37 (m, 1H), 3.04-2.96 (m, 1H), 2.87-2.71 (m, 3H), 2.67-2.53 (m, 1H), 2.42 (q, J=11.6 Hz, 2H), 2.19-2.10 (m, 1H), 1.63-1.55 (m, 2H), 1.51 (s, 9H).

### Step 4—3-[5-methoxy-3-methyl-2-oxo-4-(4-piperidyl)benzimidazol-1-yl]piperidine-2,6-dione

To a solution of tert-butyl 4-[5-methoxy-1-[1-[(4-methoxyphenyl)methyl]-2,6-dioxo-3-piperidyl]-3-methyl-2-oxo-benzimidazol-4-yl]piperidine-1-carboxylate (1.53 g, 2.58 mmol) in TFA (8 mL) was added TfOH (3.40 g, 22.6 mmol). The reaction was stirred at 70° C. for 4 hrs. On completion, the reaction was concentrated in vacuo to give title compound (1.26 g, 100% yield, TFA) as brown oil. LC-MS (ESI⁺) m/z 373.3 (M+H)⁺.

### Step 5—Tert-butyl 4-[1-(2,6-dioxo-3-piperidyl)-5-methoxy-3-methyl-2-oxo-benzimidazol-4-yl] piperidine-1-carboxylate

To a solution of 3-[5-methoxy-3-methyl-2-oxo-4-(4-piperidyl)benzimidazol-1-yl]piperidine-2,6-dione (1.26 g, 2.59 mmol) and TEA (2.62 g, 25.9 mmol) in DCM (15 mL) was added Boc₂O (847 mg, 3.89 mmol) at 0° C. Then the reaction was stirred at 25° C. for 1 hr. On completion, the reaction was diluted with DCM (30 mL). The organic layer was washed with water (20 mL×3), dried over Na₂SO₄ and filtered. The filtrate was concentrated in vacuo. The residue was purified by reverse phase (0.10% FA condition) to give title compound (0.9 g, 73% yield) as white solid. ¹H NMR (400 MHz, DMSO-d₆) δ 11.08 (s, 1H), 6.92 (d, J=8.8 Hz, 1H), 6.72 (d, J=8.8 Hz, 1H), 5.37-5.29 (m, 1H), 4.08-3.95 (m, 2H), 3.73 (s, 3H), 3.59 (s, 3H), 3.52-3.43 (m, 1H), 2.94-2.76 (m, 3H), 2.71-2.61 (m, 2H), 2.28-2.17 (m, 2H), 2.02-1.93 (m, 1H), 1.59 (d, J=11.2 Hz, 2H), 1.44 (s, 9H).

### Step 6—3-[5-methoxy-3-methyl-2-oxo-4-(4-piperidyl)benzimidazol-1-yl]piperidine-2,6-dione

To a solution of tert-butyl 4-[1-(2,6-dioxo-3-piperidyl)-5-methoxy-3-methyl-2-oxo-benzimidazol-4-yl]piperidine-1-carboxylate (500 mg, 1.06 mmol) in DCM (5 mL) was added TFA (1.5 mL). The reaction was stirred at 25° C. for 1 hr. On completion, the reaction was concentrated in vacuo to give title compound (390 mg, 75% yield, TFA) as brown oil. LC-MS (ESI⁺) m/z 373.2 (M+H)⁺.

### 5-Bromo-4-(dimethylamino)-2-nitro-benzaldehyde (Intermediate BUD)

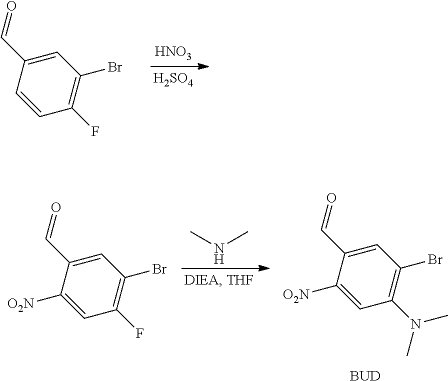

### Step 1—5-Bromo-4-fluoro-2-nitro-benzaldehyde

To a solution of 3-bromo-4-fluoro-benzaldehyde (3.00 g, 14.7 mmol, CAS #77771-02-9) in H₂SO₄ (18 mL) was added HNO₃ (2.78 g, 29.5 mmol, 1.99 mL, 67% solution) at 0° C., the reaction was stirred at 25° C. for 3 hrs. On completion, the reaction mixture was poured into ice water (50 mL), then extracted with EA (2×30 mL). The organic phase was washed with brine, dried over Na₂SO₄, filtered and the filtrate was concentrated in vacuo to give the title compound (2.20 g, 60% yield) as a white solid. ¹H NMR (400 MHz, DMSO-d₆) δ 10.14 (s, 1H), 8.31 (d, J=8.4 Hz, 1H), 8.25 (d, J=7.2 Hz, 1H).

### Step 2—5-Bromo-4-(dimethylamino)-2-nitro-benzaldehyde

To a solution of 5-bromo-4-fluoro-2-nitro-benzaldehyde (2.00 g, 8.06 mmol) and N-methylmethanamine (1.97 g, 24.1 mmol, 2.22 mL, HCl, CAS #124-40-3) in THF (20 mL) was added DIEA (5.21 g, 40.3 mmol, 7.02 mL). Then the reaction mixture was stirred at 100° C. for 12 hrs. On completion, the reaction mixture was concentrated in vacuo to give a residue. The residue was purified by reverse phase (0.1% FA condition) to give title compound (1.70 g, 77% yield) as a yellow solid. ¹H NMR (400 MHz, DMSO-d₆) δ 9.97 (s, 1H), 8.08 (s, 1H), 7.58 (s, 1H), 2.98 (s, 6H).

### N-[6-(dimethylamino)-2-(4-formylcyclohexyl)indazol-5-yl]-6-(trifluoromethyl)pyridine-2-carboxamide (Intermediate BUE)

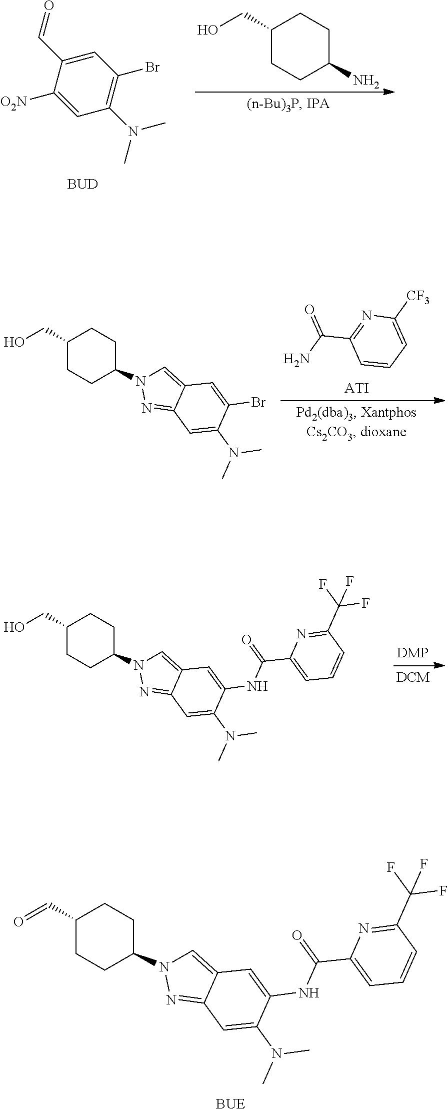

### Step 1—[4-[5-Bromo-6-(dimethylamino)indazol-2-yl]cyclohexyl]methanol

A solution of 5-bromo-4-(dimethylamino)-2-nitro-benzaldehyde (1.60 g, 5.86 mmol, Intermediate BUD) and (4-aminocyclohexyl)methanol (756 mg, 5.86 mmol, CAS #1467-84-1) in IPA (30 mL) was stirred at 80° C. for 12 hrs. Then the mixture was cooled to 25° C., tributylphosphane (3.56 g, 17.5 mmol, 4.34 mL) was added to the mixture which was then stirred at 80° C. for 6 hrs. On completion, the reaction mixture was concentrated in vacuo to give a residue. The residue was purified by reverse phase (0.1% FA condition), then purified by column chromatography (SiO₂, Petroleum ether/Ethyl acetate=10/1 to 1/2) to give the title compound (1.70 g, 82% yield) as yellow oil. LC-MS (ESI⁺) m/z 351.9 (M+H)⁺.

### Step 2—N-[6-(dimethylamino)-2-[4-(hydroxymethyl)cyclohexyl]indazol-5-yl]-6-(trifluoromethyl) pyridine-2-carboxamide

To a solution of [4-[5-bromo-6-(dimethylamino)indazol-2-yl]cyclohexyl]methanol (300 mg, 851 umol), 6-(trifluoromethyl)pyridine-2-carboxamide (178 mg, 936 umol, Intermediate ATI) in dioxane (5 mL) was added Cs₂CO₃ (554 mg, 1.70 mmol), Pd₂(dba)₃ (77.9 mg, 85.1 umol) and Xantphos (49.2 mg, 85.1 umol). Then the reaction mixture was stirred at 100° C. for 12 hrs under N₂. On completion, the reaction mixture was filtered and the filtrate was concentrated in vacuo to give a residue. The residue was purified by reverse phase (0.1% FA condition) to give the title compound (230 mg, 58% yield) as a yellow solid. ¹H NMR (400 MHz, DMSO-d₆) δ 11.04 (s, 1H), 8.71 (s, 1H), 8.50-8.43 (m, 1H), 8.43-8.37 (m, 1H), 8.35 (s, 1H), 8.20 (dd, J=0.8, 7.6 Hz, 1H), 7.44 (s, 1H), 4.49 (t, J=5.2 Hz, 1H), 4.43-4.31 (m, 1H), 3.29 (t, J=5.6 Hz, 2H), 2.74 (s, 6H), 2.20-2.08 (m, 2H), 1.97-1.80 (m, 4H), 1.55-1.40 (m, 1H), 1.23-1.05 (m, 2H); LC-MS (ESI⁺) m/z 462.0 (M+H)⁺.

### Step 3—N-[6-(dimethylamino)-2-(4-formylcyclohexyl)indazol-5-yl]-6-(trifluoromethyl)pyridine-2-carboxamide

To a solution of N-[6-(dimethylamino)-2-[4-(hydroxymethyl)cyclohexyl]indazol-5-yl]-6-(trifluoromethyl) pyridine-2-carboxamide (220 mg, 476 umol) in DCM (2 mL) was added DMP (242 mg, 572 umol, 177 uL), then the reaction mixture was stirred at 25° C. for 2 hrs. On completion, the reaction mixture was quenched with Na₂S₂O₃ (15 mL) and extracted with DCM (2×30 mL). The combined organic phase was washed with NaHCO₃ and brine (2×15 mL), dried with anhydrous Na₂SO₄, filtered and concentrated in vacuo to the title compound (210 mg, 95% yield) as a yellow solid. ¹H NMR (400 MHz, DMSO-d₆) δ 11.04 (s, 1H), 9.64 (s, 1H), 8.71 (s, 1H), 8.52-8.44 (m, 1H), 8.43-8.37 (m, 1H), 8.35 (s, 1H), 8.20 (d, J=7.8 Hz, 1H), 7.44 (s, 1H), 4.48-4.36 (m, 1H), 2.74 (s, 6H), 2.47-2.37 (m, 1H), 2.23-2.15 (m, 2H), 2.15-2.06 (m, 2H), 2.02-1.89 (m, 2H), 1.51-1.38 (m, 2H); LC-MS (ESI⁺) m/z 460.2 (M+H)⁺.

### N-[6-(difluoromethyl)-2-[3-(iodomethyl)cyclobutyl]indazol-5-yl]-6-(trifluoromethyl) pyridine-2-carboxamide (Intermediate BUF)

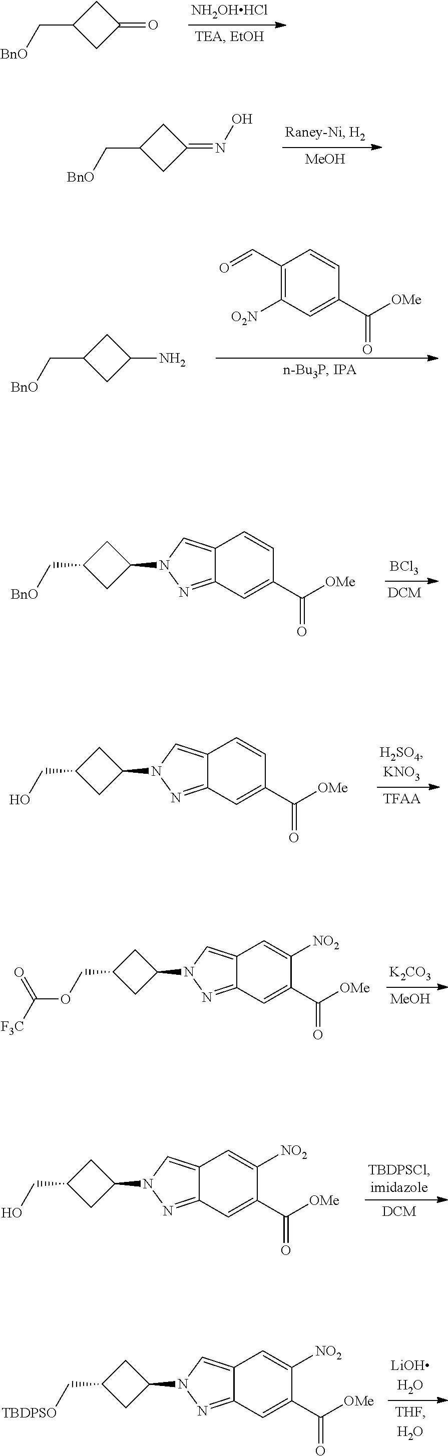

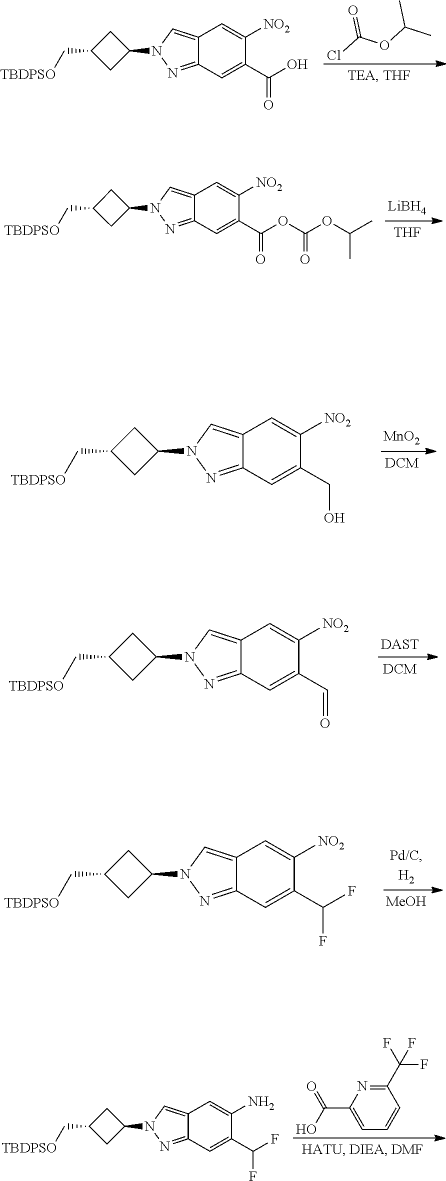

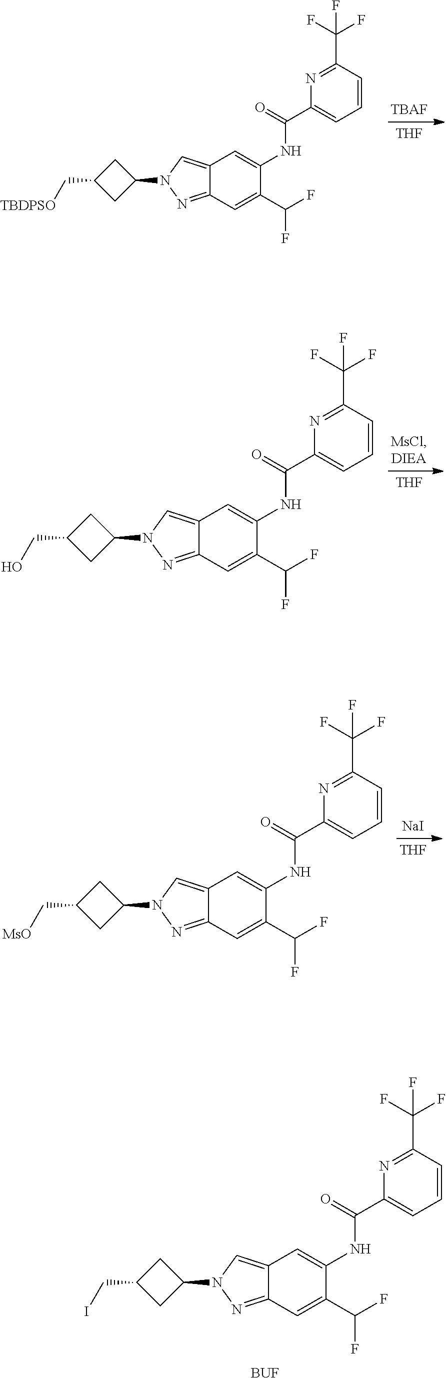

### Step 1—3-(Benzyloxymethyl)cyclobutanone oxime

To a solution of 3-(benzyloxymethyl)cyclobutanone (10 g, 52.5 mmol, CAS #172324-67-3) in EtOH (100 mL) was added TEA (5.32 g, 52.5 mmol) and NH₂OH·HCl (3.65 g, 52.5 mmol). The mixture was then stirred at 40° C. for 16 hrs. On completion, the reaction mixture was quenched with water (100 mL) and extracted with ethyl acetate (2×200 mL). The combined organic layers were dried over Na₂SO₄, filtered and the filtrate was concentrated under reduced pressure to give the title compound (10.0 g, 92% yield) as colorless oil. ¹H NMR (400 MHz, CHLOROFORM-d) δ 7.44-7.27 (m, 5H), 4.55 (s, 2H), 3.53 (d, J=6.0 Hz, 2H), 3.11-2.95 (m, 2H), 2.75-2.64 (m, 3H). LC-MS (ESI⁺) m/z 206.1 (M+H)⁺.

### Step 2—3-(Benzyloxymethyl)cyclobutanamine

To a solution of 3-(benzyloxymethyl)cyclobutanone oxime (10 g, 48.7 mmol) in MeOH (100 mL) was added Raney-Ni (417 mg, 4.87 mmol) at 25° C. under nitrogen atmosphere. After addition, the mixture was stirred at 50° C. for 4 hrs under H₂ atmosphere (50 psi). On completion, the mixture was filtrated and the filtrate was concentrated under reduced pressure. The filtrate was concentrated under reduced pressure to give the title compound (9.00 g, 96% yield) as colorless oil. LC-MS (ESI⁺) m/z 192.2 (M+H)⁺.

### Step 3—Methyl 2-[3-(benzyloxymethyl)cyclobutyl]indazole-6-carboxylate

To a solution of methyl 4-formyl-3-nitro-benzoate (9.84 g, 47.0 mmol, CAS #153813-69-5) in IPA (180 mL) was added 3-(benzyloxymethyl)cyclobutanamine (9 g, 47.0 mmol) at 25° C. After addition, the mixture was stirred at 80° C. for 4 hrs. Next, tributylphosphane (28.5 g, 141 mmol, CAS #998-40-3) was added dropwise at 0° C. The resulting mixture was stirred at 80° C. for 16 hrs. On completion, the mixture was concentrated under reduced pressure to afford a residue and the residue was purified by column chromatography (SiO₂, petroleum ether/ethyl acetate=10/1 to 2/1) to give the title compound (4.20 g, 24% yield) as yellow oil. LC-MS (ESI⁺) m/z 351.3 (M+H)⁺.

### Step 4—Methyl 2-[3-(hydroxymethyl)cyclobutyl]indazole-6-carboxylate

To a solution of methyl 2-[3-(benzyloxymethyl)cyclobutyl]indazole-6-carboxylate (4.2 g, 11.9 mmol) in DCM (30 mL) was added BCL₃ (1 M in CDM, 23.97 mL) at 0° C. Then the mixture was stirred at 25° C. for 4 hrs. On completion, the mixture was quenched with water (50 mL) and extracted with DCM (3×80 mL). The organic layer was dried over Na₂SO₄ and concentrated under reduced pressure to give the title compound (3.00 g, 96% yield) as a yellow solid. ¹H NMR (400 MHz, MeOD) δ 8.42-8.33 (m, 2H), 7.77-7.73 (m, 1H), 7.68-7.63 (m, 1H), 5.30-5.21 (m, 1H), 3.93 (s, 3H), 3.74 (d, J=6.4 Hz, 2H), 2.88-2.77 (m, 2H), 2.70-2.59 (m, 1H), 2.56-2.48 (m, 2H).

### Step 5—Methyl 5-nitro-2-((1r, 3r)-3-((2,2,2-trifluoroacetoxy)methyl)cyclobutyl)-2H-indazole-6-carboxylate

A mixture of methyl 2-[3-(hydroxymethyl)cyclobutyl]indazole-6-carboxylate (3.00 g, 11.5 mmol) in TFAA (75.5 g, 359 mmol) was stirred at 25° C. for 1 hr. Then the reaction mixture was cool to 0° C. and H₂SO₄ (6.78 g, 69.1 mmol) was added at 0° C. Then KNO₃ (2.07 g, 20.4 mmol) was added to the reaction mixture at 0° C. and the reaction mixture was stirred at 0° C. for 2 hrs. On completion, the reaction mixture was poured into water (150 mL, 0° C.). The mixture was extracted with EA (3×200 mL) and the organic layer was washed with NaHCO₃ aqueous until the pH=8. The combined organic layer was concentrated under reduced pressure to afford a residue. The residue was purified by column chromatography (SiO₂, petroleum ether/ethyl acetate=5/1 to 1/1) to give the title compound (3.50 g, 84% yield) as a yellow solid. ¹H NMR (400 MHz, CDCl₃) δ 8.41 (s, 1H), 8.24-8.22 (m, 1H), 8.08 (s, 1H), 5.27-5.18 (m, 1H), 4.56 (d, J=5.6 Hz, 2H), 3.95 (s, 3H), 3.15-2.97 (m, 3H), 2.65-2.59 (m, 2H).

### Step 6—Methyl 2-[3-(hydroxymethyl)cyclobutyl]-5-nitro-indazole-6-carboxylate

To a solution of methyl 5-nitro-2-((1r, 3r)-3-((2,2,2-trifluoroacetoxy)methyl)cyclobutyl)-2H-indazole-6-carboxylate (3.3 g, 10.8 mmol) in MeOH (50 mL) was added K₂CO₃ (2.69 g, 19.4 mmol). The mixture was stirred at 25° C. for 2 hrs. On completion, water (30 mL) was added to the reaction mixture and the mixture was extracted with EA (3×80 mL). The organic layer was dried over Na₂SO₄ and filtered. The filtrate was concentrated under reduced pressure to give the title compound (2.51 g, 100% yield) as yellow oil. LC-MS (ESI⁺) m/z 306.1 (M+H)⁺.

### Step 7—Methyl 2-[3-[[tert-butyl(diphenyl)silyl]oxymethyl]cyclobutyl]-5-nitro-indazole-6-carboxylate

To a mixture of methyl 2-[3-(hydroxymethyl)cyclobutyl]-5-nitro-indazole-6-carboxylate (3.00 g, 9.83 mmol), and tert-butyl-chloro-diphenyl-silane (3.24 g, 11.7 mmol) in DMF (30 mL) was added imidazole (2.01 g, 29.4 mmol) and tert-butyl-chloro-diphenyl-silane (3.24 g, 11.7 mmol) at 0° C. The mixture was then stirred at 25° C. for 3 hrs under N₂ atmosphere. On completion, the mixture was quenched with H₂O (20 mL) and extracted with EA (3×100 mL). The organic layer was concentrated under reduced pressure to afford a residue. The residue was purified by column chromatography (SiO₂, petroleum ether/ethyl acetate=10/1 to 2/1) to give the title compound (2.50 g, 37% yield) as a yellow solid. LC-MS (ESI⁺) m/z 544.4 (M+H)⁺.

### Step 8—2-[3-[[Tert-butyl(diphenyl)silyl]oxymethyl]cyclobutyl]-5-nitro-indazole-6-carboxylic acid

To a solution of methyl 2-[3-[[tert-butyl(diphenyl)silyl]oxymethyl]cyclobutyl]-5-nitro-indazole-6-carboxylate (2.6 g, 4.78 mmol) in THF (40 mL) and H₂O (10 mL) was added LiOH·H₂O (1.00 g, 23.9 mmol). The mixture was then stirred at 50° C. for 16 hrs. On completion, the mixture was concentrated under reduced pressure to remove the THF and the mixture was adjusted pH=3 with HCl (1 M). Then the mixture was extracted with EA (3×100 mL) and dried over Na₂SO₄. The organic layer was filtered and the filtrate was concentrated under reduced pressure to give the title compound (2.50 g, 77% yield) as a yellow solid. LC-MS (ESI⁺) m/z 530.1 (M+H)⁺.

### Step 9—Isopropoxycarbonyl 2-[3-[[tert-butyl(diphenyl)silyl]oxymethyl]cyclobutyl]-5-nitro-indazole-6-carboxylate

To a solution of 2-[3-[[tert-butyl(diphenyl)silyl]oxymethyl]cyclobutyl]-5-nitro-indazole-6-carboxylic acid (2.50 g, 4.72 mmol) in THF (50 mL) was added TEA (1.43 g, 14.1 mmol) and isopropyl carbonochloridate (578 mg, 4.72 mmol, CAS #108-23-6). Then the mixture was stirred at 25° C. for 1 hr. On completion, the reaction mixture was concentrated under reduced pressure to give the title compound (2.50 g, 86% yield) as yellow liquid. LC-MS (ESI⁺) m/z 616.2 (M+H)⁺.

### Step 10—[2-[3-[[Tert-butyl(diphenyl)silyl]oxymethyl]cyclobutyl]-5-nitro-indazol-6-yl]methanol

A mixture of isopropoxycarbonyl 2-[3-[[tert-butyl(diphenyl)silyl]oxymethyl]cyclobutyl]-5-nitro-indazole-6-carboxylate (2.50 g, 4.06 mmol) in THF (50 mL) was added LiBH₄ (770 mg, 35.3 mmol) at 0° C. under nitrogen atmosphere. The mixture was degassed and purged with N₂ three times, and then the mixture was stirred at 0° C. for 2 hrs under N₂ atmosphere. On completion, the reaction was quenched with H₂O (15 mL) at 0° C. and the mixture was extracted with EA (2×150 mL). The organic layers were combined and dried over Na₂SO₄. The organic layer was concentrated under reduced pressure to afford a residue. The residue was purified by column chromatography (SiO₂, petroleum ether/ethyl acetate=5/1 to 1/1) to give the title compound (2.00 g, 88% yield) as a yellow solid. ¹H NMR (400 MHz, CDCl₃-d) δ 8.63 (s, 1H), 8.20 (s, 1H), 7.94 (s, 1H), 7.73-7.70 (m, 4H), 7.47-7.40 (m, 6H), 5.26-5.17 (m, 1H), 5.01 (s, 2H), 3.83 (d, J=4.8 Hz, 2H), 2.90-2.83 (m, 2H), 2.76-2.70 (m, 1H), 2.68-2.61 (m, 2H), 2.38 (s, 1H), 1.13 (s, 9H). LC-MS (ESI⁺) m/z 516.3 (M+H)⁺.

### Step 11—2-[3-[[Tert-butyl(diphenyl)silyl]oxymethyl]cyclobutyl]-5-nitro-indazole-6-carbaldehyde

To a solution of [2-[3-[[tert-butyl(diphenyl)silyl]oxymethyl]cyclobutyl]-5-nitro-indazol-6-yl]methanol (1.90 g, 3.68 mmol) in DCM (20 mL) was added MnO₂ (6.41 g, 73.6 mmol). Then the mixture was stirred at 25° C. for 2 hrs. On completion, the reaction mixture was filtered through celatom and the filtrate was concentrated under reduced pressure to give the title compound (1.80 g, 95% yield) as a yellow solid. LC-MS (ESI⁺) m/z 514.2 (M+H)⁺.

### Step 12—Tert-butyl-[[3-[6-(difluoromethyl)-5-nitro-indazol-2-yl]cyclobutyl]methoxy]-diphenyl-silane

To a stirred solution of 2-[3-[[tert-butyl(diphenyl)silyl]oxymethyl]cyclobutyl]-5-nitro-indazole-6-carbaldehyde (900 mg, 1.75 mmol) in DCM (15 mL) under nitrogen atmosphere was added DAST (1.13 g, 7.01 mmol) at 0° C. Then the reaction mixture was stirred at 25° C. for 16 hrs under nitrogen atmosphere. On completion, the reaction was quenched with cold water (1 mL) and extracted with DCM (3×50 mL). The organic layer was washed with saturated sodium chloride (50 mL), dried over sodium sulfate, filtered and concentrated under reduced pressure to give the title compound (900 mg, 95% yield) as a yellow solid. LC-MS (ESI⁺) m/z 536.2 (M+H)⁺.

### Step 13—2-[3-[[Tert-butyl(diphenyl)silyl]oxymethyl]cyclobutyl]-6-(difluoromethyl)indazol-5-amine

A mixture of tert-butyl-[[3-[6-(difluoromethyl)-5-nitro-indazol-2-yl]cyclobutyl]methoxy]-diphenyl-silane (800 mg, 1.49 mmol) and Pd/C (880 mg, 746 umol, 10 wt %) in MeOH (15 mL) was degassed and purged with H₂ three 3 times, and then the mixture was stirred at 25° C. for 3 hrs under H₂ atmosphere (15 psi). On completion, the reaction mixture was filtered through celatom and the filter cake was washed with MeOH (3×20 mL). The filtrate was concentrated under reduced pressure to give the title compound (700 mg, 78% yield) as a yellow solid. LC-MS (ESI⁺) m/z 506.3 (M+H)⁺.

### Step 14—N-[2-[3-[[tert-butyl(diphenyl)silyl]oxymethyl]cyclobutyl]-6-(difluoromethyl)indazol-5-yl]-6-(trifluoromethyl)pyridine-2-carboxamide

To a solution of 6-(trifluoromethyl)pyridine-2-carboxylic acid (661 mg, 3.46 mmol, CAS #131747-42-7) in DMF (10 mL) was added DIEA (536 mg, 4.15 mmol) and HATU (1.58 g, 4.15 mmol) and the mixture was stirred at 25° C. for 1 hr. Then the mixture was added to a solution of 2-[3-[[tert-butyl(diphenyl) silyl]oxymethyl]cyclobutyl]-6-(difluoromethyl)indazol-5-amine (700 mg, 1.38 mmol) in DMF (10 mL) at 25° C. and the mixture was stirred at 25° C. for 1 hr. On completion, the reaction mixture was quenched with H₂O (0.5 mL) and concentrated under reduced pressure to afford a residue. The residue was purified by column chromatography (SiO₂, petroleum ether/ethyl acetate=10/1 to 1/1) to give the title compound (750 mg, 74% yield) as a yellow solid. ¹HNMR(400 MHz, CHLOROFORM-d) δ 10.53 (s, 1H), 8.73 (s, 1H), 8.51 (d, J=7.6 Hz, 1H), 8.18-8.12 (m, 1H), 8.03 (s, 1H), 7.94 (s, 1H), 7.91-7.88 (m, 1H), 7.73-7.70 (m, 4H), 7.47-7.40 (m, 6H), 6.97-6.68 (m, 1H), 5.27-5.16 (m, 1H), 3.83 (d, J=5.2 Hz, 2H), 2.93-2.83 (m, 2H), 2.77-2.69 (m, 1H), 2.67-2.60 (m, 2H), 1.13 (s, 8H). LC-MS (ESI⁺) m/z 679.3 (M+H)⁺.

### Step 15—N-[6-(difluoromethyl)-2-[3-(hydroxymethyl)cyclobutyl]indazol-5-yl]-6-(trifluoromethyl) pyridine-2-carboxamide

To a solution of N-[2-[3-[[tert-butyl(diphenyl)silyl]oxymethyl]cyclobutyl]-6-(difluoromethyl)indazol-5-yl]-6-(trifluoromethyl)pyridine-2-carboxamide (750 mg, 1.10 mmol) in THF (10 mL) was added TBAF (1 M in THF, 1.33 mL) at 0° C. Then the mixture was stirred at 25° C. for 2 hrs. On completion, the reaction mixture was concentrated under reduced pressure to afford a residue. The residue was purified by column chromatography (SiO₂, petroleum ether/ethyl acetate=10/1 to 1/1) to give the title compound (450 mg, 92% yield) as yellow oil. ¹H NMR (400 MHz, MeOD-d₄) δ 8.54 (s, 1H), 8.48 (d, J=7.6 Hz, 1H), 8.41 (s, 1H), 8.31 (t, J=7.6 Hz, 1H), 8.07 (d, J=7.6 Hz, 1H), 7.93 (s, 1H), 7.17-6.88 (m, 1H), 5.49 (s, 1H), 5.32-5.22 (m, 1H), 3.75 (d, J=6.4 Hz, 2H), 2.88-2.80 (m, 2H), 2.72-2.60 (m, 1H), 2.57-2.50 (m, 2H).

### Step 16—[3-[6-(Difluoromethyl)-5-[[6-(trifluoromethyl)pyridine-2-carbonyl]amino]indazol-2-yl] cyclobutyl]methyl methanesulfonate

To a solution of N-[6-(difluoromethyl)-2-[3-(hydroxymethyl)cyclobutyl]indazol-5-yl]-6-(trifluoromethyl)pyridine-2-carboxamide (200 mg, 454 umol) in DCM (4 mL) was added TEA (68.9 mg, 681 umol) and methanesulfonyl chloride (410 mg, 3.58 mmol) at 0° C. The mixture was then stirred at 25° C. for 16 hrs. On completion, the reaction mixture was quenched by H₂O (2 mL) and extracted with DCM (3×50 mL). The combined organic layer was washed with brine (60 mL), dried over Na₂SO₄ and filtered. The filtrate was concentrated under reduced pressure to give the title compound (250 mg, 88% yield) as a yellow solid. LC-MS (ESI⁺) m/z 519.2 (M+H)⁺.

### Step 17—N-[6-(difluoromethyl)-2-[3-(iodomethyl)cyclobutyl]indazol-5-yl]-6-(trifluoromethyl) pyridine-2-carboxamide

A mixture of [3-[6-(difluoromethyl)-5-[[6-(trifluoromethyl)pyridine-2-carbonyl]amino]indazol-2-yl]cyclobutyl]methyl methanesulfonate (200 mg, 385 umol) and iodosodium (578 mg, 3.86 mmol) in acetone (10 mL) was degassed and purged with N₂ three times, and then the mixture was stirred at 60° C. for 4 hrs under N₂ atmosphere. On completion, to the mixture was added water (00 mL) and the solution was extracted with EA (3×50 mL). The organic layer was washed with brine (2×30 mL), dried over Na₂SO₄ and filtered. The filtrate was concentrated under reduced pressure to give the title compound (200 mg, 94% yield) as a yellow solid. LC-MS (ESI⁺) m/z 551.2 (M+H)⁺.

### 3-fluoro-N-[2-(4-formylcyclohexyl)-6-methoxy-indazol-5-yl]-5-(trifluoromethyl) benzamide (Intermediate BTO)

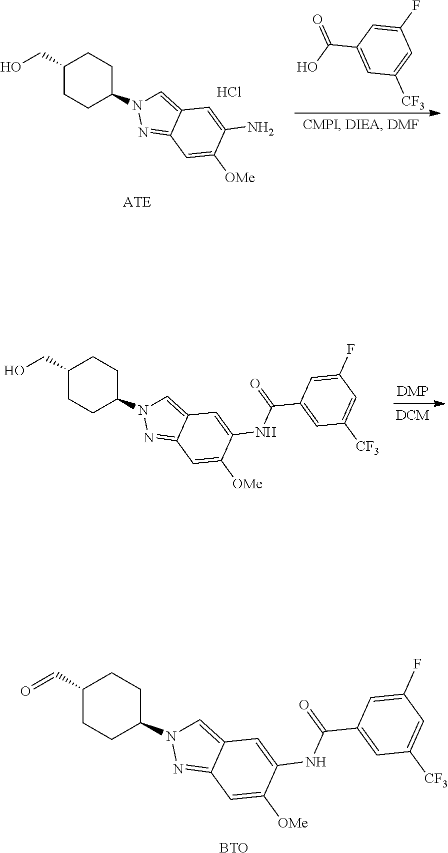 [SMILES: COc1cc2nn([C@H]3CC[C@H](CO)CC3)cc2cc1N ; Cl ; O=C(O)c1cc(F)cc(C(F)(F)F)c1 ; COc1cc2nn([C@H]3CC[C@H](CO)CC3)cc2cc1NC(=O)c1cc(F)cc(C(F)(F)F)c1 ; COc1cc2nn([C@H]3CC[C@H](C=O)CC3)cc2cc1NC(=O)c1cc(F)cc(C(F)(F)F)c1 ; "BTO"]

### Step 1—3-Fluoro-N-[2-[4-(hydroxymethyl) cyclohexyl]-6-methoxy-indazol-5-yl]-5-(trifluoromethyl) benzamide

To a solution of 3-fluoro-5-(trifluoromethyl) benzoic acid (146 mg, 705 umol, CAS #161622-05-5) in DMF (3 mL) was added CMPI (180 mg, 705 umol) and DIEA (248 mg, 1.92 mmol), then the mixture was stirred at 25° C. for 5 mins. Next, a solution of [4-(5-amino-6-methoxy-indazol-2-yl)cyclohexyl]methanol (200 mg, 641 umol, HCl, Intermediate ATE) in DMF (2 mL) was added to above mixture, and the reaction was stirred at 25° C. for 1 hrs. On completion, the reaction mixture was quenched by water (0.05 mL), and diluted with EtOAc (30 mL). The organic layer was washed with brine (2×10 mL). The organic layer was separated, dried over Na₂SO₄ and concentrated in vacuo to give the title compound (210 mg, 70.34% yield) as a brown solid. LC-MS (ESI⁺) m/z 466.4 (M+H)⁺.

### Step 2—3-Fluoro-N-[2-(4-formylcyclohexyl)-6-methoxy-indazol-5-yl]-5-(trifluoromethyl)benzamide

To a solution of 3-fluoro-N-[2-[4-(hydroxymethyl)cyclohexyl]-6-methoxy-indazol-5-yl]-5-(trifluoromethyl)benzamide (150 mg, 322 umol) in DCM (5 mL) was added DMP (177 mg, 418 umol) at 25° C., then the mixture was stirred at 25° C. for 2 hrs. On completion, the reaction mixture was quenched with Na₂S₂O₃ solution (3 mL), diluted with DCM (20 mL), and then washed with NaHCO₃ (2×15 mL). The organic layer was separated, dried over Na₂SO₄ and concentrated in vacuo to give the title compound (0.149 g, 79% yield) as a brown solid. LC-MS (ESI⁺) m/z 464.1 (M+H)⁺.

### N-[2-[3-(hydroxymethyl)cyclobutyl]indazol-5-yl]-6-(trifluoromethyl)pyridine-2-carboxamide (Intermediate BUI)

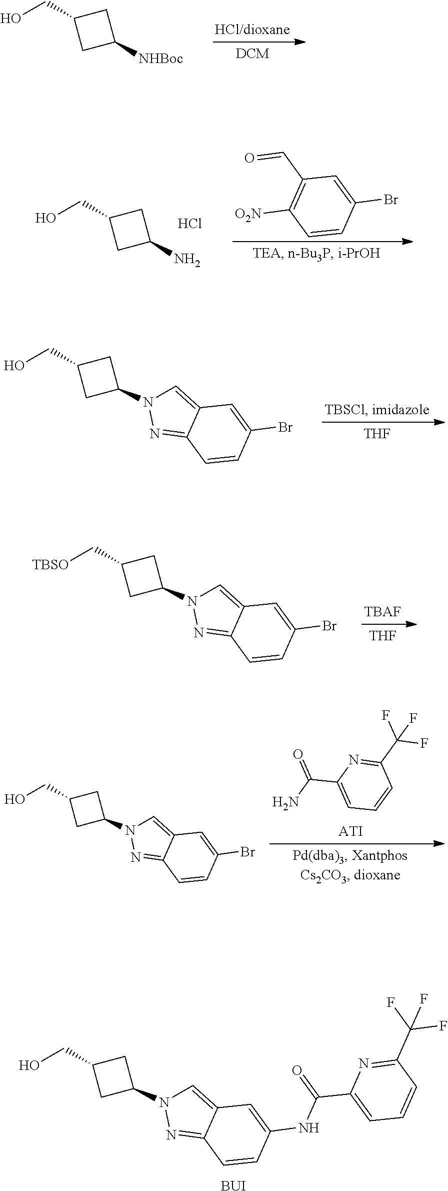

### Step 1—(3-Aminocyclobutyl)methanol

To a solution of tert-butyl N-[3-(hydroxymethyl)cyclobutyl]carbamate (5.30 g, 26.3 mmol; CAS #167081-37-0) in DCM (30 mL) was added HCl/dioxane (4 M, 100 mL) at 25° C., and the mixture was stirred 25° C. for 16 hr. On completion, the reaction mixture was concentrated in vacuo to give the title compound (3.50 g, 96%) as colorless oil. ¹H NMR (400 MHz, DMSO-d₆) δ8.23 (s, 2H), 3.74-3.59 (m, 2H), 3.44-3.36 (m, 2H), 2.38 (d, J=4.4, 8.9 Hz, 1H), 2.21-1.87 (m, 4H).

### Step 2—[3-(5-Bromoindazol-2-yl)cyclobutyl]methanol

To a solution of (3-aminocyclobutyl)methanol (3.14 g, 22.8 mmol) in i-PrOH (50 mL) was added Et₃N (7.70 g, 76.0 mmol) at 0° C. The mixture was stirred at 0° C. for 0.5 hrs, then 5-bromo-2-nitro-benzaldehyde (3.5 g, 15.2 mmol, CAS #20357-20-4) was added to the mixture and stirred at 25° C. for 2 hrs. Next, tributylphosphane (9.24 g, 45.6 mmol) was added to the mixture and warmed up to 80° C. and stirred for 2 hrs. On completion, the reaction mixture was poured into 50 mL of water and extracted with EtOAc (100 mL×2). The combined organic layers were washed by saturated brine (100 mL), dried over Na₂SO₄, filtered and concentrated in vacuo to give a crude product. The crude product was purified by column chromatography (SiO₂, PE/EA=50:1 to EA) to give the title compound (3.60 g, 84% yield) as yellow oil. LC-MS (ESI⁺) m/z 280.9 (M+H)⁺.

### Step 3—[3-(5-Bromoindazol-2-yl)cyclobutyl]methoxy-tert-butyl-dimethyl-silane

To a solution of [3-(5-bromoindazol-2-yl)cyclobutyl]methanol (3.00 g, 10.6 mmol) in THF (20 mL) was added TBSCI (1.93 g, 12.8 mmol), imidazole (1.09 g, 16.0 mmol) at 25° C., then the mixture was stirred at 25° C. for 16 hrs. On completion, the reaction mixture was poured into 50 mL of water and extracted with EtOAc (100 mL×2). The combined organic layers were washed with saturated brine (100 mL), dried over Na₂SO₄, filtered and concentrated to give a crude product. The crude product was purified by column chromatography (SiO₂, Petroleum ether/Ethyl acetate=100/1 to 50/1) to give the title compound (1.00 g, 23% yield) as yellow solid. ¹H NMR (400 MHz, CDCl₃) δ 7.91 (s, 1H), 7.80 (d, J=0.8 Hz, 1H), 7.63 (d, J=9.2 Hz, 1H), 7.33 (dd, J=1.2, 9.4 Hz, 1H), 5.08-5.16 (m, 1H), 3.77 (d, J=4.8 Hz, 2H), 2.89-2.74 (m, 2H), 2.64 (td, J=4.4, 9.2 Hz, 1H), 2.60-2.48 (m, 2H), 0.96 (s, 9H), 0.12 (s, 6H).

### Step 4—[3-(5-Bromoindazol-2-yl)cyclobutyl]methanol

To a solution of [3-(5-bromoindazol-2-yl)cyclobutyl]methoxy-tert-butyl-dimethyl-silane (1.00 g, 2.53 mmol) in THF (15 mL) was added TBAF (1 M, 2.78 mL) and stirred at 25° C. for 1 hr. On completion, the mixture was poured into water (40 mL) and extracted with EA (20 mL×2). The combined organic layers were dried over Na₂SO₄, filtered and concentrated in vacuo to give a residue. The residue was purified by silica gel column chromatography (petroleum ether/ethyl acetate=100:1, 5:1) to give the title compound (650 mg, 91% yield) as white solid. ¹H NMR (400 MHz, CDCl₃) δ 7.92 (s, 1H), 7.80 (d, J=1.2 Hz, 1H), 7.62 (d, J=9.2 Hz, 1H), 7.34 (d, J=9.2 Hz, 1H), 5.10-5.18 (m, 1H), 3.84 (d, J=6.4 Hz, 2H), 2.93-2.83 (m, 2H), 2.71 (d, J=2.8, Hz, 1H), 2.62-2.47 (m, 2H).

### Step 5—N-[2-[3-(hydroxymethyl)cyclobutyl]indazol-5-yl]-6-(trifluoromethyl)pyridine-2-carboxamide

To a solution of [3-(5-bromoindazol-2-yl)cyclobutyl]methanol (0.45 g, 1.60 mmol) in dioxane (8 mL) was added 6-(trifluoromethyl)pyridine-2-carboxamide (365 mg, 1.92 mmol, Intermediate ATI), Cs₂CO₃ (1.04 g, 3.20 mmol)Pd₂(dba)₃ (146 mg, 160 umol), and ditert-butyl-[2-(2,4,6-triisopropylphenyl) phenyl]phosphane (67.9 mg, 160 umol) at 25° C. under N₂. Then the mixture was stirred at 100° C. for 16 hrs. On completion, the mixture was diluted with water (20 mL) and extracted with EA (20 mL×2). The combined organic layers were dried over Na₂SO₄, filtered and concentrated in vacuo to give a residue. The residue was purified by silica gel chromatography (petroleum ether/ethyl acetate=100:1 to 1/3) to give the title compound (350 mg, 56% yield) as yellow solid. ¹H NMR (400 MHz, DMSO-d₆) δ 10.37 (s, 1H), 8.44 (s, 1H), 8.42-8.33 (m, 2H), 8.29 (s, 1H), 8.17 (d, J=7.2 Hz, 1H), 7.66-7.53 (m, 2H), 5.16-5.24 (m, 1H), 4.76 (t, J=5.2 Hz, 1H), 3.58 (t, J=6.0 Hz, 2H), 2.81-2.61 (m, 2H), 2.48-2.44 (m, 1H), 2.41-2.34 (m, 2H).

### N-[6-(1-hydroxy-1-methyl-ethyl)-2-[4-(iodomethyl)cyclohexyl]indazol-5-yl]-6-(trifluoro methyl)pyridine-2-carboxamide (Intermediate BUJ)

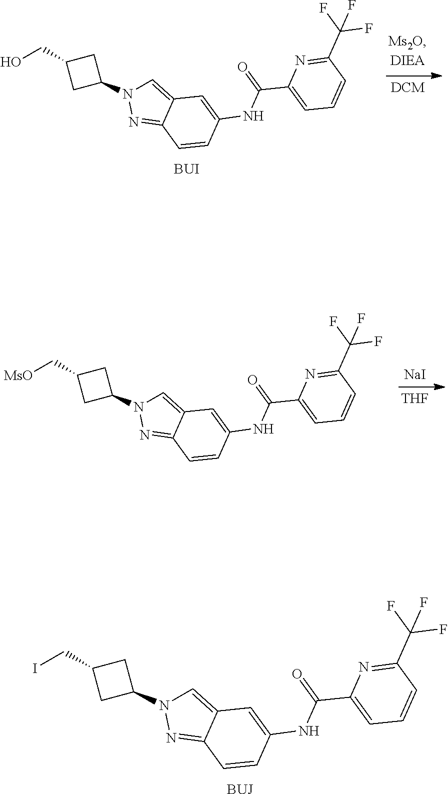

### Step 1—[3-[5-[[6-(Trifluoromethyl)pyridine-2-carbonyl]amino]indazol-2-yl]cyclobutyl]methyl methanesulfonate

To a solution of N-[2-[3-(hydroxymethyl)cyclobutyl]indazol-5-yl]-6-(trifluoromethyl)pyridine-2-arboxamide (0.06 g, 153 umol, Intermediate BUI) in THF (2 mL) was added methylsulfonyl methanesulfonate (80.3 mg, 461 umol) and DIEA (79.4 mg, 614 umol). Then the mixture was stirred at 25° C. for 2 hrs. On completion, the reaction mixture was poured into 10 mL of water and extracted with EtOAc (10 mL×2). The combined organic layers were washed with saturated brine (10 mL), dried over Na₂SO₄, filtered and concentrated in vacuo to give the title compound (68.0 mg, 94% yield) as yellow solid. ¹H NMR (400 MHz, CDCl₃) δ 9.92 (s, 1H), 8.64-8.48 (m, 2H), 8.26-8.11 (m, 2H), 8.05-7.87 (m, 2H), 7.59 (d, J=9.2 Hz, 1H), 5.48 (s, 1H), 4.43 (d, J=3.2 Hz, 2H), 3.14 (s, 3H), 2.96 (s, 2H), 2.80-2.62 (m, 2H), 1.46-1.39 (m, 1H).

### Step 2—N-[6-(1-hydroxy-1-methyl-ethyl)-2-[4-(iodomethyl)cyclohexyl]indazol-5-yl]-6-(trifluoro methyl)pyridine-2-carboxamide

To a solution of [3-[5-[[6-(trifluoromethyl)pyridine-2-carbonyl]amino]indazol-2-yl]cyclobutyl]methyl methanesulfonate (66.0 mg, 140 umol) in THF (2 mL) was added NaI (95.0 mg, 634 umol). Then the mixture was stirred at 65° C. for 16 hrs. On completion, the reaction mixture was poured into 10 mL of water and extracted with EtOAc (10 mL×2). The combined organic layers were washed by saturated brine (10 mL), dried over Na₂SO₄, filtered and concentrated to give the title compound (65 mg, 92% yield) as yellow solid. ¹H NMR (400 MHz, DMSO-d₆) δ 10.37 (s, 1H), 8.44 (s, 1H), 8.41-8.33 (m, 2H), 8.29 (d, J=1.2 Hz, 1H), 8.17 (dd, J=1.2, 7.6 Hz, 1H), 7.69-7.54 (m, 2H), 5.28 (q, J=7.2 Hz, 1H), 3.62-3.57 (m, 2H), 2.96-2.83 (m, 1H), 2.76-2.66 (m, 2H), 2.38-2.26 (m, 2H).

### 5-Chloro-N-[2-(4-formylcyclohexyl)-6-methoxy-indazol-5-yl]pyridine-3-carboxamide (Intermediate BUK)

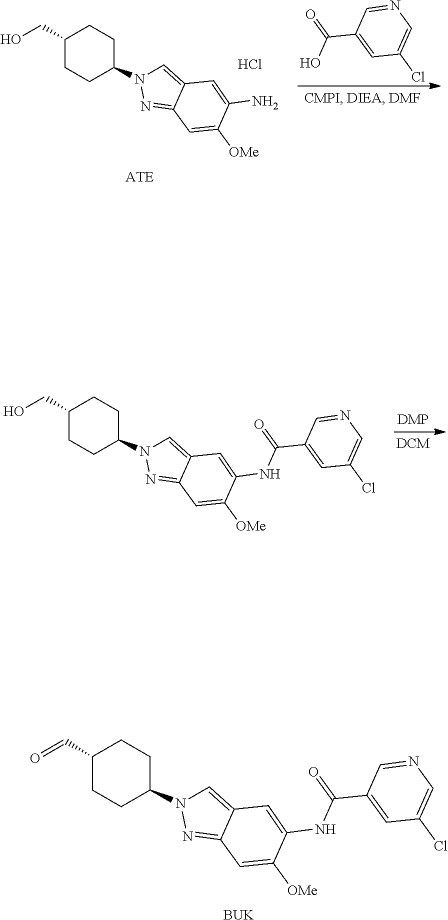

### Step 1—5-Chloro-N-[2-[4-(hydroxymethyl)cyclohexyl]-6-methoxy-indazol-5-yl]pyridine-3-carboxamide

To a solution of [4-(5-amino-6-methoxy-indazol-2-yl)cyclohexyl]methanol (200 mg, 726 umol, Intermediate ATE) in DMF (6 mL) was added CMPI (167 mg, 653 umol), 5-chloropyridine-3-carboxylic acid (103 mg, 653 umol, CAS #22620-27-5) and DIEA (375 mg, 2.91 mmol) in DMF (6 mL). The mixture was stirred at 25° C. for 1 hr. On completion, the mixture concentrated under reduced pressure to give a residue. The residue was purified by prep-HPLC (0.1% FA condition) to give the title compound (220 mg, 73% yield) as off-white solid. LC-MS (ESI+) m/z 415.3 (M+H)⁺.

### Step 2—5-Chloro-N-[2-(4-formylcyclohexyl)-6-methoxy-indazol-5-yl]pyridine-3-carboxamide

To a solution of 5-chloro-N-[2-[4-(hydroxymethyl)cyclohexyl]-6-methoxy-indazol-5-yl]pyridine-3-carboxamide (50.0 mg, 120 umol) in DCM (1 mL) was added DMP (61.3 mg, 144 umol). The mixture was stirred at 25° C. for 2 hrs. On completion, the reaction mixture was quenched with Na₂S₂SO₃ (10 mL) aqueous and NaHCO₃ aqueous (10 mL) at 25° C. Then the solution was diluted with NaHCO₃ aqueous (15 mL) and extracted with DCM (10 mL×2). The combined organic layers were dried over anhydrous Na₂SO₄, filtered and concentrated under reduced pressure to give the crude product (49.0 mg, 98% yield) as a yellow solid. LC-MS (ESI+) m/z 413.3 (M+H)⁺.

### 5-Chloro-N-[2-(4-formylcyclohexyl)-6-methoxy-indazol-5-yl]pyridine-3-carboxamide (Intermediate BUL)

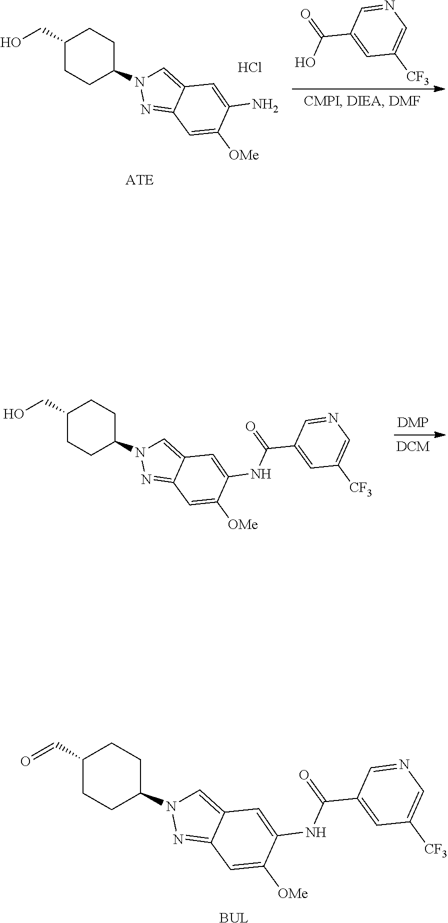

### Step 1—N-[2-[4-(hydroxymethyl)cyclohexyl]-6-methoxy-indazol-5-yl]-5-(trifluoromethyl)pyridine-3-carboxamide

To a solution of [4-(5-amino-6-methoxy-indazol-2-yl)cyclohexyl]methanol (150 mg, 544 umol, Intermediate ATE) in DMF (1 mL) was added CMPI (125 mg, 490 umol), 5-(trifluoromethyl)pyridine-3-carboxylic acid (93.7 mg, 490 umol, CAS #131747-40-5) and DIEA (281 mg, 2.18 mmol) in DMF (1 mL). The mixture was stirred at 25° C. for 1 hr. On completion, the mixture was concentrated in vacuo. The residue was purified by prep-HPLC (0.1% FA condition) to give the title compound (60.0 mg, 24% yield) as off-white solid. LC-MS (ESI+) m/z 449.3 (M+H)⁺.

### Step 2—5-Chloro-N-[2-(4-formylcyclohexyl)-6-methoxy-indazol-5-yl]pyridine-3-carboxamide

To a solution of N-[2-[4-(hydroxymethyl)cyclohexyl]-6-methoxy-indazol-5-yl]-5-(trifluoromethyl) pyridine-3-carboxamide (48.0 mg, 107 umol) in DCM (1 mL) was added DMP (54.4 mg, 128 umol). The mixture was stirred at 25° C. for 2 hrs. On completion, the mixture was quenched with Na₂S₂SO₃ aqueous (10 mL) and NaHCO₃ aqueous (10 mL) at 25° C., and then the mixture was extracted with DCM (10 mL×2). The combined organic layers were dried over anhydrous Na₂SO₄, filtered and concentrated in vacuo to give the crude product (35.0 mg, 73% yield) as a yellow solid. LC-MS (ESI+) m/z 447.3 (M+H)⁺.

### N-[2-(4-formylcyclohexyl)-6-methoxy-indazol-5-yl]pyrrolo[2,3-b]pyridine-1-carboxamide (Intermediate BUM)

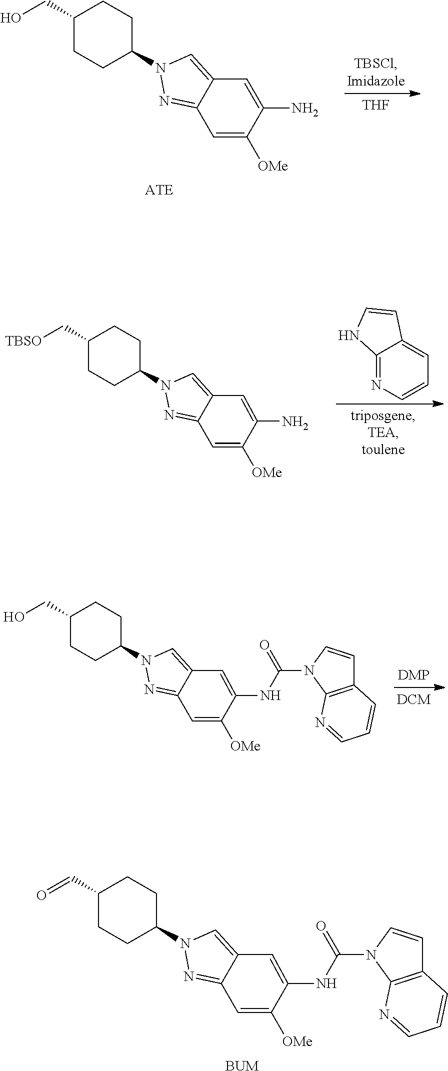

### Step 1—2-[4-[[Tert-butyl(dimethyl)silyl]oxymethyl]cyclohexyl]-6-methoxy-indazol-5-amine

To a solution of [4-(5-amino-6-methoxy-indazol-2-yl)cyclohexyl]methanol (400 mg, 1.28 mmol, HCl, Intermediate ATE) in THF (10 mL) was added Imidazole (174 mg, 2.57 mmol) and TBSCI (232 mg, 1.54 mmol), then the reaction mixture was stirred at 25° C. for 2 hrs. On completion, the reaction mixture was quenched with H₂O (15 mL) and extracted with DCM (2×30 mL). The combined organic phase was brine (2×15 mL), dried with anhydrous Na₂SO₄, filtered and concentrated in vacuo to give title compound (495 mg, 99% yield) as a yellow solid. LC-MS (ESI⁺) m/z 390.7 (M+H)⁺.

### Step 2—N-[2-[4-(hydroxymethyl)cyclohexyl]-6-methoxy-indazol-5-yl]pyrrolo[2,3-b]pyridine-1-carboxamide

To a solution of 1H-pyrrolo[2,3-b]pyridine (150 mg, 1.27 mmol, CAS #517918-95-5) and TEA (642 mg, 6.35 mmol) in ACN (5 mL) was added triphosgene (226 mg, 761 umol). The mixture was stirred at 0° C. for 0.5 hr. Next, 2-[4-[[tert-butyl(dimethyl)silyl]oxymethyl]cyclohexyl]-6-methoxy-indazol-5-amine (494 mg, 1.27 mmol) was added to the mixture, and the reaction mixture was stirred at 25° C. for 12 hrs. On completion, the reaction mixture was quenched with NaHCO₃ (20 mL), then extracted with DCM (2×30 mL). The organic phase was dried over Na₂SO₄, filtered and the filtrate was concentrated in vacuo to give a residue. The residue was purified by reverse phase (0.1% FA condition) to give title compound (100 mg, 18% yield) as a brown solid. ¹H NMR (400 MHz, DMSO-d₆) δ 12.34 (s, 1H), 8.55 (dd, J=1.2, 4.8 Hz, 1H), 8.50 (s, 1H), 8.31 (s, 1H), 8.22 (dd, J=1.2, 7.6 Hz, 1H), 8.09 (d, J=4.0 Hz, 1H), 7.41 (dd, J=4.8, 8.0 Hz, 1H), 7.14 (s, 1H), 6.81 (d, J=4.0 Hz, 1H), 4.49 (t, J=4.8 Hz, 1H), 4.41-4.29 (m, 1H), 4.05 (s, 3H), 3.30-3.26 (m, 2H), 2.14 (dd, J=3.2, 12.4 Hz, 2H), 1.96-1.85 (m, 4H), 1.53-1.41 (m, 1H), 1.20-1.08 (m, 2H); LC-MS (ESI⁺) m/z 420.0 (M+H)⁺.

### Step 3—N-[2-(4-formylcyclohexyl)-6-methoxy-indazol-5-yl]pyrrolo[2,3-b]pyridine-1-carboxamide

To a solution of N-[2-[4-(hydroxymethyl)cyclohexyl]-6-methoxy-indazol-5-yl]pyrrolo[2,3-b]pyridine-1-carboxamide (95 mg, 226 umol) in DCM (0.5 mL) was added DMP (115 mg, 271 umol), then the reaction mixture was stirred at 25° C. for 2 hrs. On completion, the reaction mixture was quenched with Na₂S₂O₃ (15 mL) and extracted with DCM (2×30 mL). The combined organic phase was washed with NaHCO₃ and brine (2×15 mL), dried with anhydrous Na₂SO₄, filtered and concentrated in vacuo to give the title compound (90 mg, 95% yield) as a yellow solid. ¹H NMR (400 MHz, DMSO-d₆) δ 12.34 (s, 1H), 9.64 (s, 1H), 8.55 (dd, J=1.2, 4.8 Hz, 1H), 8.51 (s, 1H), 8.31 (s, 1H), 8.22 (dd, J=1.2, 8.0 Hz, 1H), 8.09 (d, J=4.0 Hz, 1H), 7.41 (dd, J=4.8, 8.0 Hz, 1H), 7.14 (s, 1H), 6.81 (d, J=4.0 Hz, 1H), 4.43-4.35 (m, 1H), 4.05 (s, 3H), 2.45-2.38 (m, 1H), 2.21 (dd, J=3.2, 12.8 Hz, 2H), 2.14-2.07 (m, 2H), 2.02-1.91 (m, 2H), 1.46-1.43 (m, 2H); LC-MS (ESI⁺) m/z 418.1 (M+H)⁺.

### 5-cyanopyridine-3-carboxylic acid (CAS #887579-62-6) (Intermediate BUN)

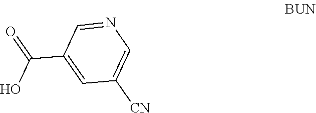 [SMILES: N#Cc1cncc(C(=O)O)c1]

### Tert-butyl N-[2-(4-formylcyclohexyl)indazol-5-yl]carbamate (Intermediate BUO)

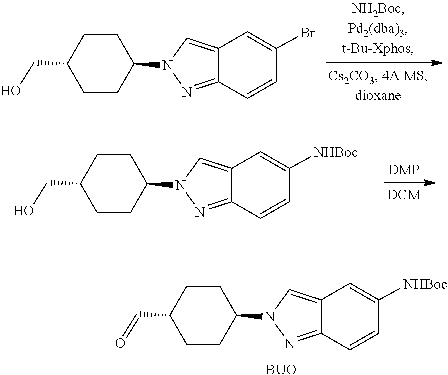

### Step 1—Tert-butyl N-[2-[4-(hydroxymethyl)cyclohexyl]indazol-5-yl]carbamate

To a solution of [4-(5-bromoindazol-2-yl)cyclohexyl]methanol (1.00 g, 3.23 mmol, synthesized via Step 1 of Intermediate BTW) and tert-butyl carbamate (378 mg, 3.23 mmol) in dioxane (15 mL), was added Cs₂CO₃ (2.11 g, 6.47 mmol), Pd₂(dba)₃ (296 mg, 323 umol), 4 Å molecular sieves (250 mg, 323 umol), and t-Bu Xphos (274 mg, 646 umol) to the mixture. The mixture was then stirred at 90° C. for 16 hrs. On completion, the reaction mixture was filtered and the filtrate was concentrated in vacuo to give a residue. The residue was purified by reverse phase (0.1% FA condition) to give the title compound (500 mg, 44% yield) as a white solid. ¹H NMR (400 MHz, DMSO-d₆) δ 9.19 (s, 1H), 8.23 (s, 1H), 7.82 (s, 1H), 7.47 (d, J=9.2 Hz, 1H), 7.19 (dd, J=2.0, 9.2 Hz, 1H), 4.48 (t, J=5.2 Hz, 1H), 4.42-4.29 (m, 1H), 3.28 (t, J=6.0 Hz, 2H), 2.17-2.07 (m, 2H), 1.95-1.83 (m, 4H), 1.48 (m, 10H), 1.21-1.06 (m, 2H), LC-MS (ESI⁺) m/z 346.6 (M+H)⁺.

### Step 2—Tert-butyl N-[2-(4-formylcyclohexyl)indazol-5-yl]carbamate

To a solution of tert-butyl N-[2-[4-(hydroxymethyl)cyclohexyl]indazol-5-yl]carbamate (190 mg, 550 umol) in DCM (1.5 mL) was added DMP (279 mg, 660 umol, 204 uL), then the reaction mixture was stirred at 25° C. for 2 hrs. On completion, the reaction mixture was quenched with Na₂S₂O₃ (10 mL) and NaHCO₃ (10 mL) and extracted with DCM (2×20 mL). The combined organic phase was washed with NaHCO₃ (15 mL) and brine (2×15 mL), dried with anhydrous Na₂SO₄, filtered and the filtrate was concentrated in vacuo to give the title compound (188 mg, 99% yield) as a purple solid. LC-MS (ESI⁺) m/z 344.2 (M+H)⁺.

### 3-[4-[1-[[4-(5-Aminoindazol-2-yl)cyclohexyl]methyl]-4-piperidyl]-3-methyl-2-oxo-benzimidazol-1-yl]piperidine-2,6-dione (Intermediate BUP)

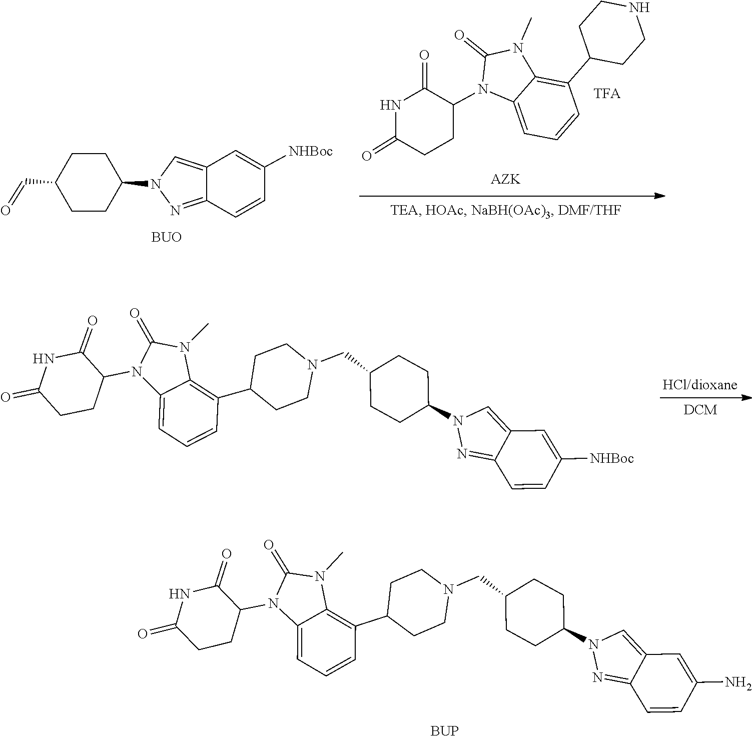

### Step 1—Tert-butyl N-[2-[4-[[4-[1-(2,6-dioxo-3-piperidyl)-3-methyl-2-oxo-benzimidazol-4-yl]-1-piperidyl]methyl]cyclohexyl]indazol-5-yl]carbamate

To a solution of 3-[3-methyl-2-oxo-4-(4-piperidyl)benzimidazol-1-yl]piperidine-2,6-dione (179 mg, 393 umol, TFA, Intermediate AZK) in THF (1.5 mL) and DMF (1.5 mL) was added TEA (39.7 mg, 393 umol, 54.7 uL), and the mixture was stirred at −10° C. for 10 mins. Then tert-butyl N-[2-(4-formylcyclohexyl)indazol-5-yl]carbamate (135 mg, 393 umol, Intermediate BUO) and AcOH (23.6 mg, 393 umol, 22.4 uL) was added to the mixture, and the mixture was stirred at −10° C. for 20 mins. Finally, NaBH(OAc)₃ (99.9 mg, 471 umol) was added to the mixture, and the mixture was stirred at −10° C. for 1 hr. On completion, the reaction mixture was quenched with water (0.5 mL) and concentrated in vacuo to give a residue. The residue was purified by reverse phase (0.1% FA condition) to give the title compound (80 mg, 30.0% yield) as a brown solid. ¹H NMR (400 MHz, DMSO-d₆)) δ 11.10 (s, 1H), 9.20 (s, 1H), 8.24 (s, 1H), 7.84 (s, 1H), 7.48 (d, J=9.2 Hz, 1H), 7.20 (dd, J=2.0, 9.2 Hz, 1H), 7.07-6.96 (m, 3H), 5.38 (dd, J=5.2, 12.4 Hz 1H), 4.49-4.35 (m, 1H), 3.61 (s, 3H), 3.49-3.38 (m, 3H), 2.88-2.82 (m, 1H), 2.72-2.58 (m, 5H), 2.20-2.10 (m, 2H), 2.06-1.88 (m, 10H), 1.87-1.80 (m, 1H), 1.48 (s, 9H), 1.29-1.20 (m, 2H), LC-MS (ESI⁺) m/z 670.4 (M+H)⁺.

### Step 2—3-[4-[1-[[4-(5-Aminoindazol-2-yl)cyclohexyl]methyl]-4-piperidyl]-3-methyl-2-oxo-benzimidazol-1-yl]piperidine-2,6-dione

To a solution of tert-butyl N-[2-[4-[[4-[1-(2,6-dioxo-3-piperidyl)-3-methyl-2-oxo-benzimidazol-4-yl]-1-piperidyl]methyl]cyclohexyl]indazol-5-yl]carbamate (50.0 mg, 74.6 umol) in DCM (1 mL) was added HCl/dioxane (4 M, 1 mL), then the reaction mixture was stirred at 25° C. for 2 hrs. On completion, the reaction mixture was concentrated in vacuo to give the title compound (45 mg, 99% yield, HCl) as white solid. LC-MS (ESI⁺) m/z 570.5 (M+H)⁺.

### 6-(5-cyanopyrrolo[2,3-b]pyridin-1-yl)-4-(isopropylamino)pyridine-3-carboxylic acid (Intermediate BUQ)

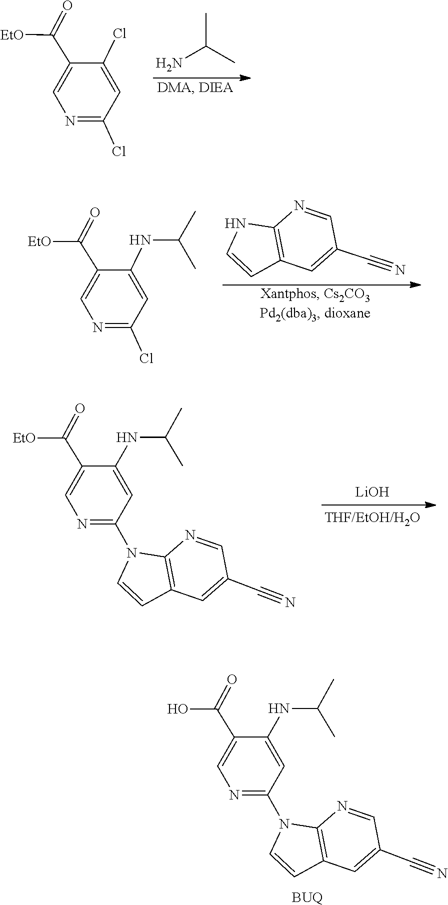

### Step 1—Ethyl 6-chloro-4-(isopropylamino)pyridine-3-carboxylate

To a solution of ethyl 4,6-dichloropyridine-3-carboxylate (1 g, 4.54 mmol, CAS #40296-46-6) in DMA (10 mL) was added DIEA (2.94 g, 22.7 mmol, 3.96 mL) and propan-2-amine (537 mg, 9.09 mmol, CAS #4432-77-3). The reaction mixture was stirred at 50° C. for 3 hrs. On completion, the reaction mixture was diluted with EtOAc (50 mL) and washed with water (50 mL×3). The organic layer was dried over Na₂SO₄, filtered and concentrated in vacuo. The residue was purified by column chromatography (SiO₂, petroleum ether/ethyl acetate=10/1, P1:R_{f}=0.5) to give title compound (0.968 g, 87% yield) as yellow solid. ¹H NMR (400 MHz, DMSO-d₆) δ 8.53 (s, 1H), 7.99 (d, J=6.8 Hz, 1H), 6.83 (s, 1H), 4.29 (q, J=7.2 Hz, 2H), 3.92-3.79 (m, 1H), 1.31 (t, J=7.2 Hz, 3H), 1.20 (d, J=6.4 Hz, 6H).

Step 2—Ethyl 6-(5-cyanopyrrolo[2,3-b]pyridin-1-yl)-4-(isopropylamino)pyridine-3-carboxylate To a solution of ethyl 6-chloro-4-(isopropylamino)pyridine-3-carboxylate (868 mg, 3.58 mmol) and 1H-pyrrolo[2,3-b]pyridine-5-carbonitrile (511 mg, 3.58 mmol, CAS #517918-95-5) in dioxane (9 mL) was added Xantphos (206 mg, 357 umol) and Cs₂CO₃ (2.33 g, 7.15 mmol). The reaction mixture was purged with N₂ gas several times, followed by addition of Pd₂(dba)₃ (327 mg, 357 umol), then the mixture was purged with N₂ again. The mixture was stirred at 110° C. for 16 hrs under N₂. On completion, the reaction mixture was filtered and concentrated in vacuo. The residue was purified by column chromatography (SiO₂, petroleum ether/ethyl acetate=10/1 to 5/1, P1:R_{f}=0.5) to give title compound (500 mg, 40% yield) as yellow solid. ¹H NMR (400 MHz, CDCl₃) δ 8.83 (s, 1H), 8.67 (s, 1H), 8.57 (d, J=4.0 Hz, 1H), 8.25 (d, J=12.0 Hz, 2H), 8.19 (s, 1H), 6.71 (d, J=3.6 Hz, 1H), 4.37 (q, J=7.2 Hz, 2H), 3.98-3.88 (m, 1H), 1.44 (s, 9H).

### Step 3—6-(5-cyanopyrrolo[2,3-b]pyridin-1-yl)-4-(isopropylamino)pyridine-3-carboxylic acid

To a solution of ethyl 6-(5-cyanopyrrolo[2,3-b]pyridin-1-yl)-4-(isopropylamino)pyridine-3-carboxylate (1 g, 2.86 mmol) in EtOH (2 mL), THF (8 mL) and H₂O (1.2 mL) was added LiOH·H₂O (1.20 g, 28.6 mmol). The mixture was stirred at 50° C. for 9 hrs. On completion, the reaction mixture was filtered and diluted with water (10 mL). The aqueous layer was acidified to pH 5-6 with 6 N HCl and lyophilized. The product was dissolved in DCM:MeOH=10:1 (22 mL) and filtered. The filtrate was concentrated in vacuo to give title compound (800 mg, 86% yield) as yellow solid. ¹H NMR (400 MHz, DMSO-d₆) δ 13.20-13.11 (m, 1H), 9.53-9.52 (m, 1H), 8.82 (d, J=2.0 Hz, 1H), 8.71 (s, 1H), 8.67 (d, J=2.0 Hz, 1H), 8.54 (d, J=4.0 Hz, 1H), 8.28 (d, J=7.2 Hz, 1H), 6.89 (d, J=4.0 Hz, 1H), 3.89-3.79 (m, 1H), 1.33 (d, J=6.4 Hz, 6H).

### 6-(5-cyanopyrrolo[2,3-b]pyridin-1-yl)-N-(4-formylcyclohexyl)-4-(isopropylamino)pyridine-3-carboxamide (Intermediate BUR)

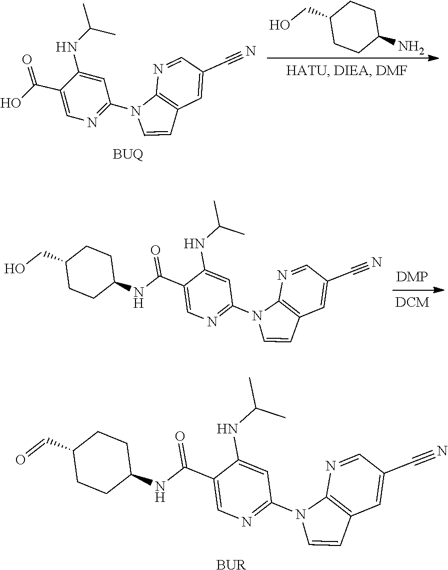

### Step 1—6-(5-cyanopyrrolo[2,3-b]pyridin-1-yl)-N-[4-(hydroxymethyl)cyclohexyl]-4-(isopropylamino) pyridine-3-carboxamide

To a solution of 6-(5-cyanopyrrolo[2,3-b]pyridin-1-yl)-4-(isopropylamino)pyridine-3-carboxylic acid (100 mg, 311 umol, Intermediate BUQ), (4-aminocyclohexyl)methanol (44.2 mg, 342 umol, CAS #1467-84-1) and DIEA (80.4 mg, 622 umol) in DMF (2 mL) was added HATU (236 mg, 622 umol). The reaction was then stirred at 25° C. for 1 hr. On completion, the reaction mixture was diluted with EtOAc (20 mL). The organic layer was washed with water (20 mL×3), dried over Na₂SO₄ and concentrated in vacuo. The residue was purified by column chromatography (SiO₂, petroleum ether/ethyl acetate=2/1 to 0/1) to give title compound (130 mg, 96% yield) as yellow solid. ¹H NMR (400 MHz, DMSO-d₆) δ 8.81 (d, J=2.0 Hz, 1H), 8.67 (d, J=2.0 Hz, 1H), 8.60 (d, J=7.2 Hz, 1H), 8.57 (s, 1H), 8.52 (d, J=4.0 Hz, 1H), 8.31 (d, J=7.6 Hz, 1H), 8.08 (s, 1H), 6.89 (d, J=4.0 Hz, 1H), 4.40 (s, 1H), 3.81-3.66 (m, 2H), 3.24 (d, J=6.0 Hz, 2H), 1.88 (d, J=9.6 Hz, 2H), 1.79 (d, J=11.6 Hz, 2H), 1.36-1.31 (m, 2H), 1.29 (d, J=6.4 Hz, 6H), 1.04-0.91 (m, 2H).

### Step 2—6-(5-cyanopyrrolo[2,3-b]pyridin-1-yl)-N-(4-formylcyclohexyl)-4-(isopropylamino)pyridine-3-carboxamide

To a solution of 6-(5-cyanopyrrolo[2,3-b]pyridin-1-yl)-N-[4-(hydroxymethyl)cyclohexyl]-4-(isopropylamino)pyridine-3-carboxamide (75 mg, 173 umol) in DCM (2 mL) was added DMP (95.6 mg, 225 umol). Then the reaction was stirred at 25° C. for 1 hr. On completion, the reaction was quenched with Na₂S₂O₃ (4 mL) and NaHCO₃ (5 mL) and the mixture was diluted with DCM (20 mL). The combined organic layers were washed with water (20 mL×3), dried over Na₂SO₄, filtered and concentrated in vacuo to give title compound (60 mg, 80 yield) as yellow solid. LC-MS (ESI⁺) m/z 431.1 (M+H)⁺.

### 1-[5-(4-formylpiperidine-1-carbonyl)-4-(isopropylamino)-2-pyridyl]pyrrolo[2,3-b]pyridine-5-carbonitrile (Intermediate BUS)

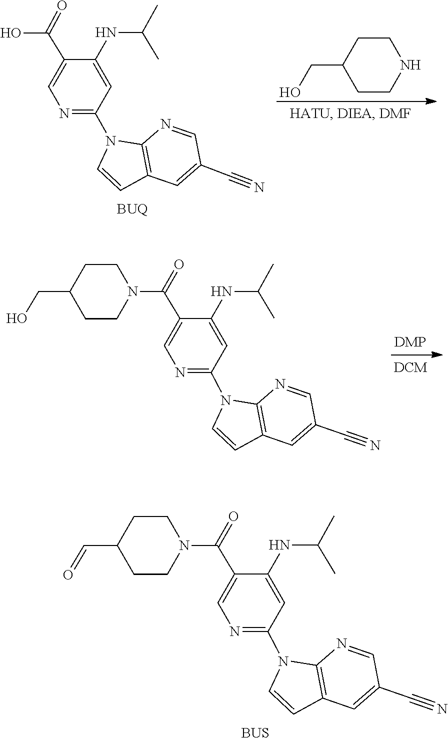

### Step 1—1-[5-[4-(hydroxymethyl)piperidine-1-carbonyl]-4-(isopropylamino)-2-pyridyl]pyrrolo[2,3-b] pyridine-5-carbonitrile

To a solution of 6-(5-cyanopyrrolo[2,3-b]pyridin-1-yl)-4-(isopropylamino)pyridine-3-carboxylic acid (100 mg, 311 umol, Intermediate BUQ), 4-piperidylmethanol (35.8 mg, 311 umol, CAS #6457-49-4) and DIEA (80.4 mg, 622 umol) in DMF (2 mL) was added HATU (130 mg, 342 umol). The reaction was stirred at 25° C. for 16 hrs. On completion, the reaction was quenched with water (10 mL) and extracted with EtOAc (20 mL×2). The organic layer was separated and concentrated in vacuo. The residue was purified by reversed-phase HPLC (0.1% FA condition) to give title compound (89 mg, 68% yield) as white solid. ¹H NMR (400 MHz, DMSO-d₆) δ 8.80 (d, J=2.0 Hz, 1H), 8.66 (d, J=2.0 Hz, 1H), 8.49 (d, J=4.0 Hz, 1H), 8.01 (d, J=10.4 Hz, 2H), 6.87 (d, J=4.0 Hz, 1H), 6.19 (d, J=7.6 Hz, 1H), 4.54-4.47 (m, 1H), 4.20-3.91 (m, 2H), 3.84-3.72 (m, 1H), 3.28 (s, 2H), 3.00-2.86 (m, 2H), 1.75-1.59 (m, 3H), 1.27 (d, J=6.4 Hz, 6H), 1.20-1.08 (m, 2H).

### Step 2—1-[5-(4-formylpiperidine-1-carbonyl)-4-(isopropylamino)-2-pyridyl]pyrrolo[2,3-b]pyridine-5-carbonitrile

To a solution of 1-[5-[4-(hydroxymethyl)piperidine-1-carbonyl]-4-(isopropylamino)-2-pyridyl]pyrrolo [2,3-b]pyridine-5-carbonitrile (85 mg, 203 umol) in DCM (2 mL) was added DMP (111 mg, 264 umol). The reaction was stirred at 25° C. for 1 hr. On completion, the reaction was quenched with Na₂S₂O₃ (5 mL) and NaHCO₃ (6 mL) solution. The mixture was diluted with DCM (20 mL) and washed with water (20 mL×3). The organic layer was separated, dried over Na₂SO₄ and filtered. The filtrate was concentrated in vacuo to give title compound (83 mg, 98% yield) as yellow solid. LC-MS (ESI⁺) m/z 417.4 (M+H)⁺.

### 5-Chloro-N-[2-(4-formylcyclohexyl)indazol-5-yl]pyridine-3-carboxamide (Intermediate BUT)

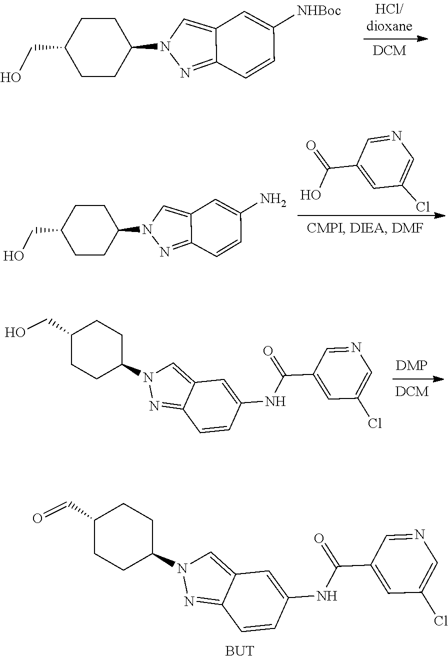

### Step 1—[4-(5-Aminoindazol-2-yl)cyclohexyl]methanol

To a solution of tert-butyl N-[2-[4-(hydroxymethyl)cyclohexyl]indazol-5-yl]carbamate (100 mg, 289 umol, synthesized via Step 1 of Intermediate BUO) in DCM (1 mL) was added in HCl/dioxane (4 M, 1 mL). The mixture was stirred for 0.5 hour at 25° C. On completion, the reaction mixture was concentrated in vacuo to give the title compound (80 mg, 98% yield, HCl) as a yellow solid. LC-MS (ESI⁺) m/z 246.0 (M+H)⁺.

### Step 2—5-Chloro-N-[2-[4-(hydroxymethyl)cyclohexyl]indazol-5-yl]pyridine-3-carboxamide

To a solution of 5-chloropyridine-3-carboxylic acid (44.7 mg, 283 umol) in DMF (1 mL) was added CMPI (87.0 mg, 340 umol) and DIEA (183 mg, 1.42 mmol, 247 uL). After addition, the mixture was stirred at 25° C. for 10 minutes, and then [4-(5-aminoindazol-2-yl)cyclohexyl]methanol (80 mg, 283 umol, HCl) in DMF (1 mL) was added dropwise at 25° C. The resulting mixture was stirred 25° C. for 2 hours. On completion, the reaction mixture was diluted with H₂O (5 mL), filtered and the filter cake was concentrated in vacuo to give the title compound (92 mg, 84% yield) as a brown solid. LC-MS (ESI⁺) m/z 385.1 (M+H)⁺.

### Step 3—5-Chloro-N-[2-(4-formylcyclohexyl)indazol-5-yl]pyridine-3-carboxamide

To a solution of 5-chloro-N-[2-[4-(hydroxymethyl)cyclohexyl]indazol-5-yl]pyridine-3-carboxamide (92 mg, 239 umol) in DCM (1 mL) was added DMP (121 mg, 286 umol). The mixture was then stirred at 25° C. for 2 hours. On completion, the reaction mixture was quenched with saturated Na₂S₂O₃ (5 mL) and saturated NaHCO₃ (5 mL), then stirred for 10 minutes. The reaction mixture was then extracted with DCM (10 mL×2). The combined organic layers were separated and washed with saturated NaCl (5 mL×2), and concentrated in vacuo to give the title compound (50 mg, 54.6% yield) as purple solid. ¹H NMR (400 MHz, DMSO-d₆) δ 10.45 (s, 1H), 9.63 (s, 1H), 9.09-9.02 (m, 1H), 8.83 (d, J=2.0 Hz, 1H), 8.50-8.40 (m, 2H), 7.66-7.58 (m, 1H), 7.45 (d, J=10.4 Hz, 1H), 4.53-4.39 (m, 1H), 3.29 (s, 2H), 2.26 -2.18 (m, 2H), 2.16-2.08 (m, 2H), 2.01-1.95 (m, 2H), 1.51-1.39 (m, 2H), 1.26-1.21 (m, 1H). LC-MS (ESI⁺) m/z 382.9 (M+H)⁺.

### N-[2-[3-(iodomethyl)cyclobutyl]pyrazolo[3,4-c]pyridin-5-yl]-6-(trifluoromethyl)pyridine-2-carboxamide (Intermediate BUU)

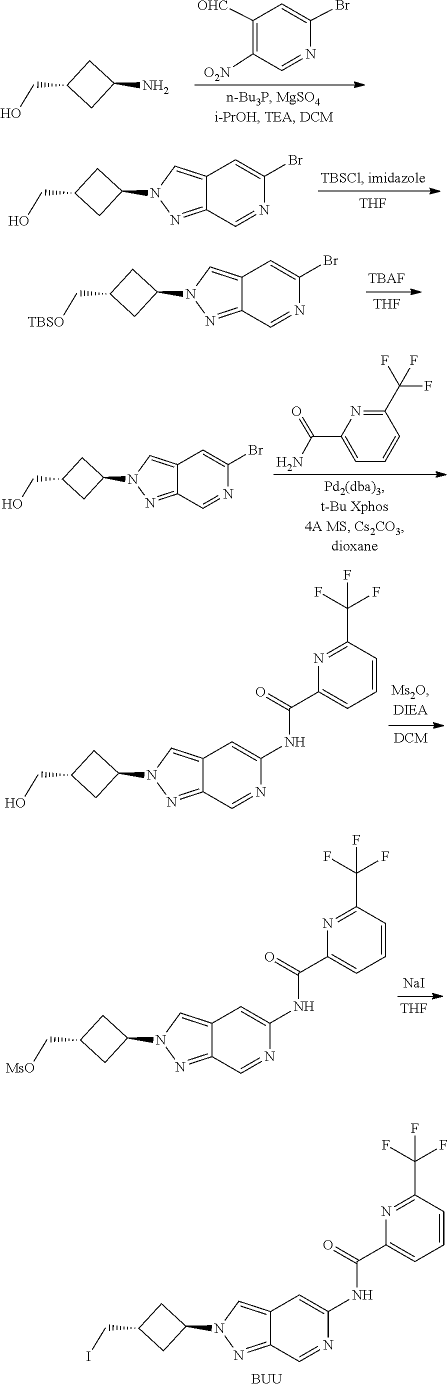

### Step 1—4-[3-(5-Bromopyrazolo[3,4-c]pyridin-2-yl)cyclobutyl]methanol

To a solution of (3-aminocyclobutyl)methanol (2.68 g, 19.4 mmol, HCl, synthesized via Step 1 of Intermediate BUI) and 2-bromo-5-nitro-pyridine-4-carbaldehyde (3.00 g, 12.9 mmol, synthesized via Steps 1-2 of Intermediate BRR) in DCM (25 mL) was added tributylphosphane (7.88 g, 38.9 mmol, 9.61 mL) and TEA (3.94 g, 38.9 mmol, 5.42 mL) at 0° C. The mixture was stirred at 25° C. for 2 hrs. Next, MgSO₄ (2.34 g, 19.4 mmol) and IPA (25 mL) were added to the solution at 25° C. Then the mixture was stirred at 80° C. for 16 hrs. On completion, the reaction was filtered and concentrated in vacuo to give the title compound (2.50 g, 68% yield) as brown oil. LC-MS (ESI⁺) m/z 282.2 (M+H)⁺.

### Step 2—[3-(5-Bromopyrazolo[3,4-c]pyridin-2-yl)cyclobutyl]methoxy-tert-butyl-dimethyl-silane

To a solution of [3-(5-bromopyrazolo[3,4-c]pyridin-2-yl)cyclobutyl]methanol (2.50 g, 8.86 mmol) in THF (20 mL) was added imidazole (1.21 g, 17.7 mmol) and TBSCl (1.74 g, 11.5 mmol, 1.41 mL). The reaction mixture was stirred at 25° C. for 16 hrs. On completion, the reaction mixture was concentrated in vacuo to give a residue. The residue was purified by column chromatography to give the title compound (1.60 g, 45% yield) as a brown solid. ¹H NMR (400 MHz, DMSO-d₆) δ 9.03 (s, 1H), 8.59 (s, 1H), 7.94 (d, J=0.8 Hz, 1H), 5.39-5.28 (m, 1H), 3.74 (d, J=6.0 Hz, 2H), 2.76-2.66 (m, 2H), 2.63-2.53 (m, 1H), 2.45-2.36 (m, 2H), 0.91 (s, 9H), 0.09 (s, 6H), LC-MS (ESI⁺) m/z 396.9 (M+H)⁺.

### Step 3—[3-(5-Bromopyrazolo[3,4-c]pyridin-2-yl)cyclobutyl]methanol

To a solution of [3-(5-bromopyrazolo[3,4-c]pyridin-2-yl)cyclobutyl]methoxy-tert-butyl-dimethyl-silane (1.50 g, 3.78 mmol) in THF (10 mL) was added TBAF (1 M, 30.2 mL). The reaction mixture was stirred at 70° C. for 16 hrs. On completion, the mixture was diluted with water (30 mL) and extracted with EA (2×80 mL). The combined organic phase was washed with brine (50 mL), dried with anhydrous Na₂SO₄, filtered and concentrated in vacuo to give a residue. The residue was triturated with PE (3 mL) and filtered to give the title compound (850 mg, 79% yield) as a brown solid. LC-MS (ESI⁺) m/z 282.2 (M+H)⁺.

### Step 4—N-[2-[3-(hydroxymethyl)cyclobutyl]pyrazolo[3,4-c]pyridin-5-yl]-6-(trifluoromethyl)pyridine-2-carboxamide

To a solution of [3-(5-bromopyrazolo[3,4-c]pyridin-2-yl)cyclobutyl]methanol (750 mg, 2.66 mmol) and 6-(trifluoromethyl)pyridine-2-carboxamide (505 mg, 2.66 mmol, Intermediate ATI) in dioxane (10 mL) was added t-Bu Xphos (225 mg, 531 umol), Pd₂(dba)₃ (243 mg, 265 umol), Cs₂CO₃ (1.73 g, 5.32 mmol) and 4 Å molecular sieves (50.0 mg). The reaction mixture was stirred at 100° C. for 16 hrs. On completion, the reaction mixture was filtered and concentrated in vacuo to give a residue. The residue was purified by column chromatography to give the title compound (400 mg, 38% yield) as a brown solid. ¹H NMR (400 MHz, DMSO-d₆) δ 10.19 (s, 1H), 9.09 (s, 1H), 8.63 (s, 1H), 8.52-8.45 (m, 2H), 8.45-8.37 (m, 1H), 8.23 (d, J=7.6 Hz, 1H), 5.37-5.26 (m, 1H), 4.78 (t, J=5.2 Hz, 1H), 3.58 (t, J=6.0 Hz, 2H), 2.76-2.66 (m, 2H), 2.58-2.52 (m, 1H), 2.46-2.38 (m, 2H); LC-MS (ESI⁺) m/z 392.2 (M+H)⁺.

### Step 5—[3-[5-[[6-(Trifluoromethyl)pyridine-2-carbonyl]amino]pyrazolo[3,4-c]pyridin-2-yl]cyclobutyl]methyl methanesulfonate

To a solution of N-[2-[3-(hydroxymethyl)cyclobutyl]pyrazolo[3,4-c]pyridin-5-yl]-6-(trifluoromethyl) pyridine-2-carboxamide (350 mg, 894 umol) in DCM (3.5 mL) was added DIEA (346 mg, 2.68 mmol, 467 uL). The methylsulfonyl methanesulfonate (311 mg, 1.79 mmol) was added at 0° C., then the reaction mixture was stirred at 25° C. for 2 hrs. On completion, the reaction was diluted with water (8 mL) and extracted with DCM (2×10 mL). The combined organic layers were washed with brine (10 mL), dried over Na₂SO₄, filtered and concentrated in vacuo to give the title compound (415 mg, 98% yield) as brown oil. LC-MS (ESI⁺) m/z 470.2 (M+H)⁺.

### Step 6—N-[2-[3-(iodomethyl)cyclobutyl]pyrazolo[3,4-c]pyridin-5-yl]-6-(trifluoromethyl)pyridine-2-carboxamide

To a solution of [3-[5-[[6-(trifluoromethyl)pyridine-2-carbonyl]amino]pyrazolo[3,4-c]pyridin-2-yl]cyclobutyl]methyl methanesulfonate (415 mg, 884 umol) in THF (3 mL) was added NaI (596 mg, 3.98 mmol), then the reaction mixture was stirred at 70° C. for 16 hrs. On completion, the mixture was diluted with water (10 mL) and extracted with DCM (2×10 mL). The combined organic layers were washed with brine (10 mL), dried over Na₂SO₄, filtered and concentrated in vacuo to give the title compound (440 mg, 99% yield) as a brown solid. ¹H NMR (400 MHz, DMSO-d₆) δ 10.19 (s, 1H), 9.09 (s, 1H), 8.63 (s, 1H), 8.50-8.43 (m, 2H), 8.43-8.37 (m, 1H), 8.24-8.19 (m, 1H), 5.46-5.31 (m, 1H), 3.64-3.55 (m, 2H), 2.98-2.84 (m, 1H), 2.81-2.69 (m, 2H), 2.42-2.33 (m, 2H); LC-MS (ESI⁺) m/z 502.1 (M+H)⁺.

### [(E)-2-cyano-3,3-diethoxy-prop-1-enoxy]potassium (Intermediate BUV

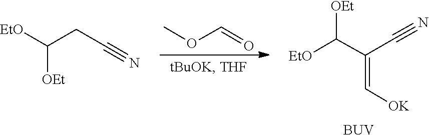

To a solution of 3,3-diethoxypropanenitrile (5.00 g, 34.9 mmol, CAS #2032-34-0) and methyl formate (2.73 g, 45.40 mmol, CAS #107-31-3) in THF (40 mL) was added potassium 2-methylpropan-2-olate (3.92 g, 34.9 mmol) slowly. The mixture was stirred at 10° C. for 2 hrs. On completion, hexane (200 mL) was added to the reaction mixture and stirred for 20 mins. Then the slurry was filtered and the cake washed with hexanes/THF (1:1) and dried at 60° C. in vacuo to give title product (4.60 g, 21.9 mmol, 62% yield) as a yellow solid. ¹H NMR (400 MHz, CD₃OD) δ=5.22 (s, 1H), 4.68 (s, 1H), 3.57 (q, J=7.2 Hz, 4H), 1.14 (t, J=7.2 Hz, 6H).

### 6-Cyanopyrazolo[1,5-a]pyrimidine-3-carboxylic acid (Intermediate BUW)

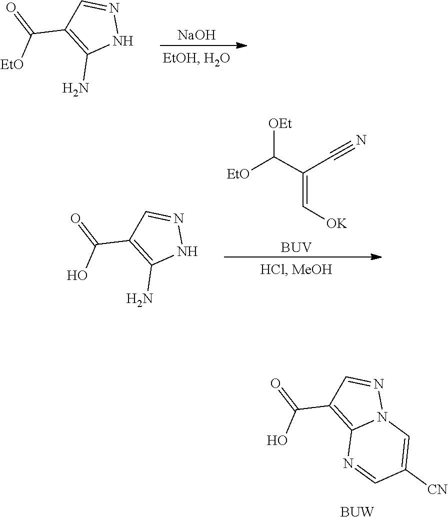

### Step 1—5-Amino-1H-pyrazole-4-carboxylic acid

To a solution of ethyl 5-amino-1H-pyrazole-4-carboxylate (5.00 g, 32.2 mmol, CAS #6994-25-8) in EtOH (25 mL) and H₂O (25 mL) was added NaOH (2.58 g, 64.4 mmol), then the mixture was stirred at 80° C. for 12 hrs. On completion, the reaction mixture was concentrated in vacuo and the residue was purified by column chromatography (SiO₂, Petroleum ether/Ethyl acetate=10:1 to 1:1) to get the title compound (4.00 g, 28.3 mmol) as a white solid. ¹HNMR (400 MHz, DMSO-d₆) δ 11.68 (s, 1H), 7.53 (s, 1H), 5.70 (s, 2H).

### Step 2—6-Cyanopyrazolo[1,5-a]pyrimidine-3-carboxylic acid

AcOH (472 mg, 7.87 mmol) was added to 5-amino-1H-pyrazole-4-carboxylic acid (50.0 mg, 393 umol) slowly and the mixture was stirred at 25° C. for 10 mins. Then a solution of [(E)-2-cyano-3,3-diethoxy-prop-1-enoxy]potassium (82.3 mg, 393 umol, Intermediate BUV) in EtOH (0.5 mL) was added. After addition, the reaction mixture was stirred at 80° C. for 2 hrs. Lots of solid was precipitated. The solid was filtered to give the title compound (50.0 mg, 265 umol) as white solid. ¹H NMR (400 MHz, DMSO-d₆) δ 10.01 (d, J=2.0 Hz, 1H), 8.89 (d, J=2.0 Hz, 1H), 8.62 (s, 1H).

### 5-chloro-6-methyl-pyridine-3-carboxylic acid (CAS #1256835-19-4) (Intermediate BUX)

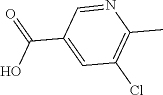

### N-[2-(3-formylcyclobutyl)-6-methoxy-indazol-5-yl]-6-(trifluoromethyl)pyridine-2-carboxamide (Intermediate BUY)

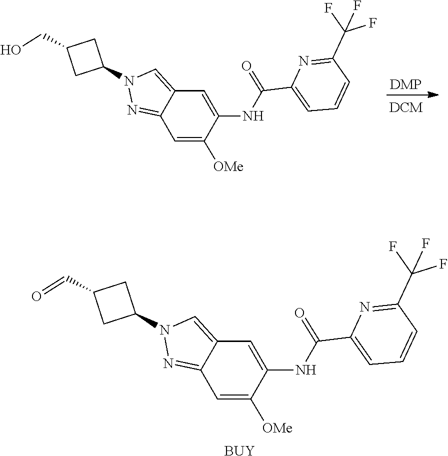

To a mixture of N-[2-[3-(hydroxymethyl) cyclobutyl]-6-methoxy-indazol-5-yl]-6-(trifluoromethyl) pyridine-2-carboxamide (50.0 mg, 118 umol, synthesized via Steps 1-3 of Intermediate BQI) in DCM (3 mL) was added DMP (75.6 mg, 178 umol). The reaction mixture was stirred at 25° C. for 12 hrs. On completion, the reaction mixture was quenched with saturated Na₂S₂O₃ (8 mL) and saturated NaHCO₃ (8 mL) at 25° C., and then stirred for 30 minutes. The mixture was extracted with DCM (2×30 mL). Then the organic layer was separated and concentrated in vacuo to give the title compound (49.0 mg, 98% yield) as yellow solid. LC-MS (ESI⁺) m/s 419.1 (M+H)⁺.

### 5-chloropyridazine-3-carboxylic acid (CAS #1211587-01-7) (Intermediate BUZ)

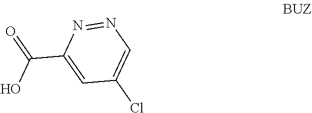

### 3-[5-Methoxy-3-methyl-2-oxo-4-(4-piperidyl)benzimidazol-1-yl]piperidine-2,6-dione (Intermediate BVA)

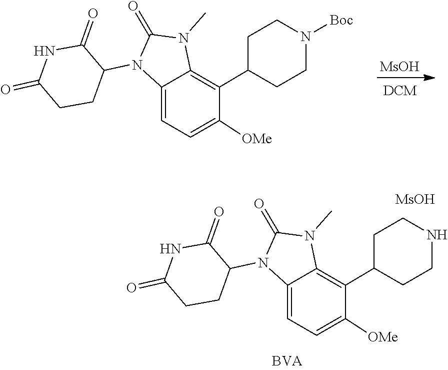

To a solution of tert-butyl 4-[1-(2,6-dioxo-3-piperidyl)-5-methoxy-3-methyl-2-oxo-benzimidazol-4-yl]piperidine-1-carboxylate (80.0 mg, 169 umol, synthesized via Steps 1-5 of Intermediate BUC) in DCM (0.5 mL) was added MsOH (48.8 mg, 507 umol). The reaction mixture was stirred at 25° C. for 30 mins. On completion, the reaction mixture was concentrated in vacuo to give the title compound (60.0 mg, 757% yield) as a gray solid. LC-MS (ESI⁺) m/z 373.3 (M+H)⁺.

### 5-Cyano-N-[2-(4-formylcyclohexyl)-6-methoxy-indazol-5-yl]pyridine-3-carboxamide (Intermediate BVB)

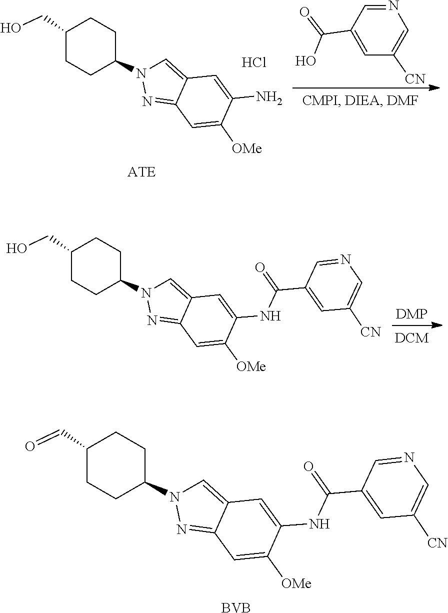

### Step 1—5-Cyano-N-[2-[4-(hydroxymethyl)cyclohexyl]-6-methoxy-indazol-5-yl]pyridine-3-carboxamide

A mixture of [4-(5-amino-6-methoxy-indazol-2-yl)cyclohexyl]methanol (260 mg, 833 umol, HCl, Intermediate ATE) and DIEA (431 mg, 3.34 mmol) in DMF (3 mL) was stirred at 25° C. for 0.2 hour. Then, 5-cyanopyridine-3-carboxylic acid (111 mg, 750 umol, CAS #887579-62-6), DIEA (431 mg, 3.34 mmol) and CMPI (276 mg, 1.08 mmol) in DMF (3 mL) was stirred at 25° C. for 0.2 hour and it was then added to the reaction mixture dropwise. The reaction mixture was stirred at 25° C. for 1.5 hours. On completion, the mixture was diluted with water (30 mL) and extracted with EA (2×30 mL). The combined organic layer was dried over Na₂SO₄, filtered and concentrated in vacuo to give a residue. The residue was purified by reverse phase (0.1% FA condition) to give the title compound (335 mg, 99% yield) as light yellow solid. ¹H NMR (400 MHz, CDCl₃) δ 9.31 (d, J=1.6 Hz, 1H), 9.05 (d, J=1.6 Hz, 1H), 8.80-8.69 (m, 2H), 8.51 (s, 1H), 7.91 (s, 1H), 7.11 (s, 1H), 4.43-4.29 (m, 1H), 4.03 (s, 3H), 3.57 (d, J=6.4 Hz, 2H), 2.39-2.32 (m, 2H), 2.10-1.96 (m, 4H), 1.73-1.64 (m, 1H), 1.26 (s, 2H). LC-MS (ESI⁺) m/z 406.2 (M+H)⁺.

### Step 2—5-Cyano-N-[2-(4-formylcyclohexyl)-6-methoxy-indazol-5-yl]pyridine-3-carboxamide

To a mixture of 5-cyano-N-[2-[4-(hydroxymethyl)cyclohexyl]-6-methoxy-indazol-5-yl]pyridine-3-carboxamide (360 mg, 887 umol) in DCM (5 mL) was added DMP (489 mg, 1.15 mmol). The reaction mixture was stirred at 25° C. for 1 hour. On completion, the reaction mixture was quenched with saturated Na₂S₂O₃ (10 mL) and saturated NaHCO₃ (10 mL) at 25° C., and then the mixture was stirred for 30 minutes. The residue was diluted with water (50 mL) and extracted with DCM (2×50 mL). The combined organic layer was dried over Na₂SO₄, filtered and concentrated in vacuo to give a residue. The residue was purified by reverse phase (0.1% FA condition) to give the title compound (250 mg, 69% yield) as yellow solid. LC-MS (ESI⁺) m/z 404.2 (M+H)⁺.

### 5-Cyano-N-[2-(4-formylcyclohexyl)-6-methoxy-indazol-5-yl]pyrrolo[2,3-b]pyridine-1-carboxamide (Intermediate BVC)

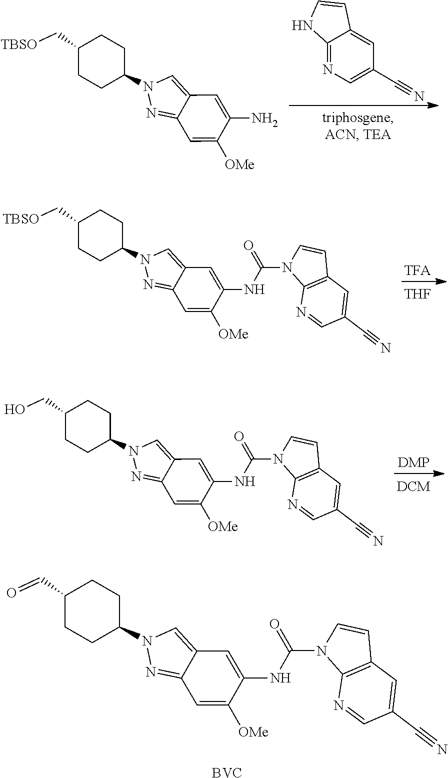

### Step 1—N-[2-[4-[[tert-butyl(dimethyl)silyl]oxymethyl]cyclohexyl]-6-methoxy-indazol-5-yl]-5-cyano-pyrrolo[2,3-b]pyridine-1-carboxamide

To a solution of 1H-pyrrolo[2,3-b]pyridine-5-carbonitrile (128 mg, 898 umol, CAS #517918-95-5) in ACN (3 mL) was added TEA (454 mg, 4.49 mmol, 625 uL), and the reaction mixture was stirred at 0° C., then triphosgene (360 mg, 1.21 mmol) was added. Next, 2-[4-[[tert-butyl (dimethyl)silyl]oxymethyl]cyclohexyl]-6-methoxy-indazol-5-amine (350 mg, 898 umol, synthesized via Step 1 of Intermediate BUM) was added, then the reaction mixture was stirred at 80° C. for 16 hrs. On completion, the mixture was quenched with water (8 mL), diluted with more water (10 mL), then extracted with DCM (2×15 mL). The combined organic layers were washed with brine (10 mL), dried over Na₂SO₄, filtered and concentrated in vacuo to give a residue. The residue was purified by reverse phase (0.1% FA condition) to give the title compound (200 mg, 39% yield) as a white solid. ¹H NMR (400 MHz, DMSO-d₆) δ 12.05 (s, 1H), 9.06 (s, 1H), 8.76 (d, J=0.8 Hz, 1H), 8.49 (s, 1H), 8.32 (s, 1H), 8.26 (d, J=4.0 Hz, 1H), 7.15 (s, 1H), 6.91 (d, J=3.6 Hz, 1H), 4.42-4.29 (m, 1H), 4.07 (s, 3H), 3.47 (d, J=6.0 Hz, 2H), 2.19-2.10 (m, 2H), 1.96-1.80 (m, 4H), 1.62-1.48 (m, 1H), 1.24-1.12 (m, 2H), 0.89 (s, 9H), 0.06 (s, 6H), LC-MS (ESI⁺) m/z 559.3 (M+H)⁺.

### Step 2—5-Cyano-N-[2-[4-(hydroxymethyl)cyclohexyl]-6-methoxy-indazol-5-yl]pyrrolo[2,3-b]pyridine-1-carboxamide

To a solution of N-[2-[4-[[tert-butyl(dimethyl)silyl]oxymethyl]cyclohexyl]-6-methoxy-indazol-5-yl]-5-cyano-pyrrolo[2,3-b]pyridine-1-carboxamide (80.0 mg, 143 umol) in THF (1 mL) was added TFA (97.9 mg, 859 umol, 63.6 uL), then the reaction mixture was stirred at 50° C. for 16 hrs. On completion, the reaction mixture was diluted with THF (3 mL), then basified with TEA until the pH=7-8. Then the mixture was extracted with DCM (2×5 mL) and the combined organic layers was dried over Na₂SO₄, filtered and concentrated in vacuo to give the title compound (60 mg, 94% yield) as a white solid. ¹H NMR (400 MHz, DMSO-d₆) δ 12.05 (s, 1H), 9.06 (d, J=2.0 Hz, 1H), 8.76 (d, J=1.6 Hz, 1H), 8.51-8.46 (m, 1H), 8.32 (s, 1H), 8.26 (d, J=4.0 Hz, 1H), 7.15 (s, 1H), 6.91 (d, J=4.0 Hz, 1H), 4.49 (t, J=5.2 Hz, 1H), 4.07 (s, 3H), 3.30-3.26 (m, 2H), 3.13-3.05 (m, 1H), 2.18-2.09 (m, 2H), 1.96-1.84 (m, 4H), 1.54-1.45 (m, 1H), 1.17-1.12 (m, 2H), LC-MS (ESI⁺) m/z 445.2 (M+H)⁺.

### Step 3—5-Cyano-N-[2-(4-formylcyclohexyl)-6-methoxy-indazol-5-yl]pyrrolo[2,3-b]pyridine-1-carboxamide

To a solution of 5-cyano-N-[2-[4-(hydroxymethyl)cyclohexyl]-6-methoxy-indazol-5-yl]pyrrolo[2,3-b]pyridine-1-carboxamide (60.0 mg, 134 umol) in DCM (1 mL) was added DMP (68.7 mg, 161 umol, 50.1 uL), then the reaction mixture was stirred at 25° C. for 2 hrs. On completion, the reaction mixture was quenched with Na₂S₂O₃ (2 mL) and NaHCO₃ (2 mL) and extracted with DCM (2×5 mL). The combined organic phase was washed with NaHCO₃ (3 mL) and brine (2×5 mL), dried with anhydrous Na₂SO₄, filtered and the filtrate was concentrated in vacuo to give the title compound (59 mg, 98% yield) as a white solid. LC-MS (ESI⁺) m/z 443.2 (M+H)⁺.

### (1R,4R)-Methyl 4-(chlorocarbonyl)cyclohexanecarboxylate (Intermediate BCU)

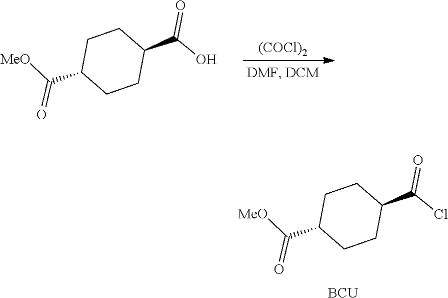

To a solution of 4-methoxycarbonylcyclohexanecarboxylic acid (500 mg, 2.69 mmol) in the DCM (10 mL) was added DMF (19.6 mg, 268 umol, 20.6 uL) and (COCl)₂ (511 mg, 4.03 mmol). The mixture was stirred at 25° C. for 1 hr. On completion, the reaction mixture was concentrated in vacuo to give the title compound (549 mg, 99% yield) as yellow oil.

### Methyl 4-(6-bromo-5-methoxy-1,3-benzothiazol-2-yl)cyclohexanecarboxylate (Intermediate

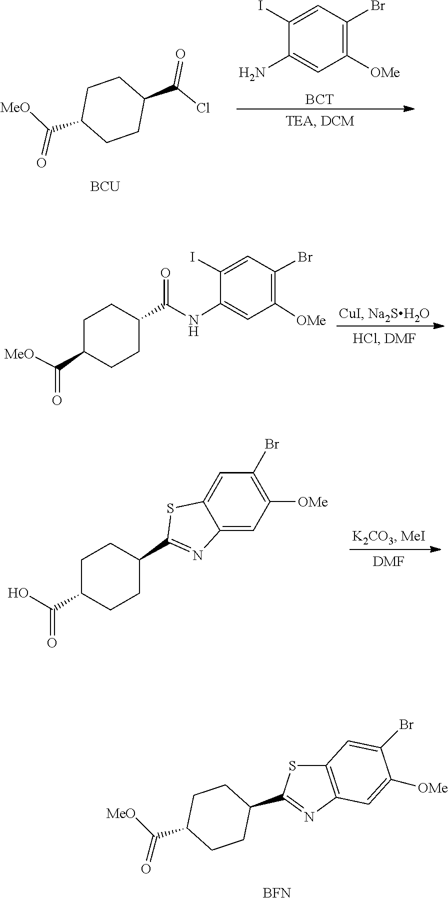

### Step 1—(1r, 4r)-Methyl 4-((4-bromo-2-iodo-5-methoxyphenyl)carbamoyl)cyclohexanecarboxylate

To a solution of 4-bromo-2-iodo-5-methoxy-aniline (880 mg, 2.68 mmol, Intermediate BCT) and Et3N (814 mg, 8.05 mmol) in the DCM (10 mL) was added methyl 4-chlorocarbonylcyclohexanecarboxylate (549 mg, 2.68 mmol, Intermediate BCU). The mixture was stirred at 25° C. for 12 hrs. On completion, the reaction mixture was washed with water (50 mL). The organic layer was dried over Na2SO4, filtered and concentrated in vacuo and the residue was triturated with (PE:EA=3:1) to give the title compound (800 mg, 60% yield) as white solid. 1H NMR (400 MHz, CDCl3) δ 8.15 (s, 1H), 7.86 (s, 1H), 7.52 (s, 1H), 3.91 (s, 3H), 3.70 (s, 3H), 2.41-2.27 (m, 2H), 2.15 (d, J=12.6 Hz, 4H), 1.69-1.49 (m, 4H).

### Step 2—(1R,4r)-4-(6-Bromo-5-hydroxybenzo[d]thiazol-2-yl)cyclohexanecarboxylic acid

To a solution of methyl 4-[(4-bromo-2-iodo-5-methoxy-phenyl)carbamoyl]cyclohexanecarboxylate (0.8 g, 1.61 mmol) in the DMF (10 mL) was added Na₂S·9H₂O (774 mg, 3.22 mmol) and CuI (61.4 mg, 322 umol). The mixture was stirred at 80° C. for 12 hrs under N₂. Then the mixture was cooled down to room temperature and HCl (12 M, 1.34 mL, 36% solution) was added. The mixture was stirred at 25° C. for 5 hrs. On completion, the reaction mixture was diluted with EA (100 mL) and washed with water (3×100 mL). The organic layer was dried over Na₂SO₄, filtered and concentrated in vacuo to give the title compound (570 mg, 99% yield) as yellow solid. LC-MS (ESI⁺) m/z 370.2 (M+H)⁺.

### Step 3—(1R,4r)-Methyl 4-(6-bromo-5-methoxybenzo[d]thiazol-2-yl)cyclohexanecarboxylate

To a solution of 4-(6-bromo-5-hydroxy-1,3-benzothiazol-2-yl)cyclohexanecarboxylic acid (567 mg, 1.59 mmol) in the DMF (10 mL) was added K₂CO₃ (440 mg, 3.19 mmol) and MeI (678 mg, 4.78 mmol). The mixture was stirred at 25° C. for 3 hrs. On completion, the reaction mixture was diluted with EA (100 mL) and washed with water (3×100 mL). The organic layer was dried over Na₂SO₄, filtered and concentrated in vacuo and purified by column chromatography (SiO2, Petroleum ether/Ethyl acetate=10/1 to 5/1) to give the title compound (320 mg, 47% yield) as white solid. ¹H NMR (400 MHz, CDCl₃) δ 8.00 (s, 1H), 7.49 (s, 1H), 3.97 (s, 3H), 3.71 (s, 3H), 3.10-3.01 (m, 1H), 2.34-2.30 (m, 2H), 2.21-2.16 (m, 2H), 2.15-2.10 (m, 1H), 1.75-1.61 (m, 4H).

### N-[2-(4-formylcyclohexyl)-5-methoxy-1,3-benzothiazol-6-yl]-6-(trifluoromethyl)pyridine-2-carboxamide (Intermediate BCN)

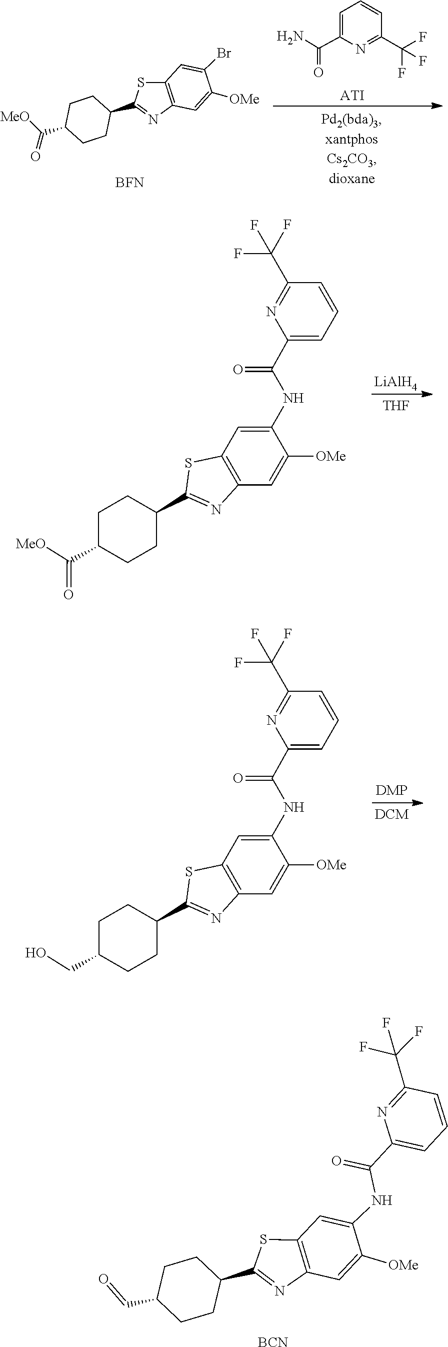

### Step 1—(1R,4r)-Methyl 4-(5-methoxy-6-(6-(trifluoromethyl)picolinamido)benzo[d]thiazol-2-yl)cyclo hexanecarboxylate

To a solution of methyl 4-(6-bromo-5-methoxy-1,3-benzothiazol-2-yl)cyclohexanecarboxylate (300 mg, 780 umol, Intermediate BFN) and 6-(trifluoromethyl)pyridine-2-carboxamide (163 mg, 858 umol, Intermediate ATI) in the dioxane (3 mL) was added Pd₂(dba)₃ (71.4 mg, 78.0 umol), Xantphos (90.3 mg, 156 umol) and Cs₂CO₃ (508 mg, 1.56 mmol). The mixture was stirred at 100° C. for 6 hrs under N₂. On completion, the mixture was concentrated in vacuo. The residue was purified by column chromatography (SiO₂, Petroleum ether/Ethyl acetate=10/1 to 3/1) to give the title compound (300 mg, 74% yield) as white solid. ¹H NMR (400 MHz, CDCl₃) δ 10.70 (s, 1H), 9.12 (s, 1H), 8.51 (d, J=8.0 Hz, 1H), 8.14 (t, J=8.0 Hz, 1H), 7.89 (d, J=7.6 Hz, 1H), 7.54 (s, 1H), 4.06 (s, 3H), 3.72 (s, 3H), 3.10-3.06 (m, 1H), 2.47-2.39 (m, 1H), 2.34 (d, J=11.2 Hz, 2H), 2.19 (d, J=11.2 Hz, 2H), 1.78-1.59 (m, 4H).

### Step 2—N-(2-((1r, 4r)-4-(hydroxymethyl)cyclohexyl)-5-methoxybenzo[d]thiazol-6-yl)-6-(trifluoro methyl)picolinamide

To a solution of methyl 4-[5-methoxy-6-[[6-(trifluoromethyl)pyridine-2-carbonyl]amino]-1,3-benzothiazol-2-yl]cyclohexanecarboxylate (50.0 mg, 101 umol) in the THF (1 mL) was added LiAlH₄ (3.85 mg, 101 umol) under 0° C. The mixture was stirred at 0° C. for 1 hr. On completion, the reaction mixture was quenched by water (0.05 mL) and NaOH (15% aq, 0.05 mL) at 0° C. Then the mixture was dried over Na₂SO₄, filtered and concentrated in vacuo to give the title compound (47.0 mg, 99% yield) as yellow solid. ¹H NMR (400 MHz, CDCl₃) δ 10.70 (s, 1H), 9.11 (s, 1H), 8.51 (d, J=7.6 Hz, 1H), 8.14 (t, J=7.6 Hz, 1H), 7.89 (d, J=7.6 Hz, 1H), 7.54 (s, 1H), 4.06 (s, 3H), 3.55 (t, J=6.0 Hz, 2H), 3.08-3.02 (m, 1H), 2.36-2.29 (m, 2H), 2.01 (dd, J=3.2, 13.2 Hz, 2H), 1.77-1.66 (m, 2H), 1.65-1.58 (m, 1H), 1.33 (t, J=5.6 Hz, 1H), 1.25-1.14 (m, 2H).

### Step 3—N-(2-((1r, 4r)-4-formylcyclohexyl)-5-methoxybenzo[d]thiazol-6-yl)-6-(trifluoromethyl) picolinamide

To a solution of N-[2-[4-(hydroxymethyl)cyclohexyl]-5-methoxy-1,3-benzothiazol-6-yl]-6-(trifluoro methyl)pyridine-2-carboxamide (47.0 mg, 100 umol) in the DCM (1 mL) was added DMP (51.4 mg, 121 umol). The mixture was stirred at 25° C. for 1 hour. On completion, the reaction mixture was quenched by the addition of Na₂S₂O₃ (aq. 3 mL) and NaHCO₃ (aq. 3 mL). Then the mixture was extracted with DCM (2×20 mL). The organic layer was dried over Na₂SO₄, filtered and concentrated in vacuo to give the title compound (46.0 mg, 98% yield) as yellow solid. LC-MS (ESI⁺) m/z 464.1 (M+H)⁺.

### Tert-butyl N-[6-fluoro-2-(4-formylcyclohexyl)indazol-5-yl]carbamate (Intermediate BVD)

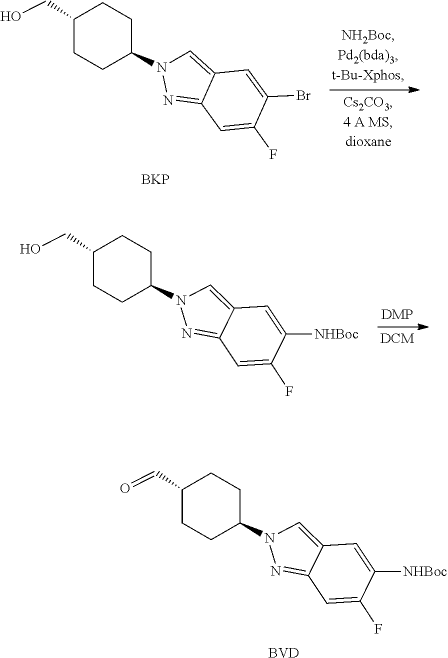

### Step 1—Tert-butyl N-[6-fluoro-2-[4-(hydroxymethyl)cyclohexyl]indazol-5-yl]carbamate

To a solution of [4-(5-bromo-6-fluoro-indazol-2-yl)cyclohexyl]methanol (1.00 g, 3.06 mmol, Intermediate BKP) and tert-butyl carbamate (358 mg, 3.06 mmol) in dioxane (10 mL), was added Pd₂(dba)₃ (279 mg, 305 umol), Cs₂CO₃ (1.99 g, 6.11 mmol), 4 Å molecular sieves (250 mg), and t-Bu Xphos (259 mg, 611 umol), then the mixture was stirred at 90° C. for 16 hrs. On completion, the mixture was concentrated in vacuo to give the residue. The residue was purified by reverse phase (0.1% FA condition) to give the title compound (600 mg, 54% yield) as a white solid. ¹H NMR (400 MHz, DMSO-d₆) δ 8.73 (s, 1H), 8.35 (s, 1H), 7.86-7.65 (m, 1H), 7.41-7.28 (m, 1H), 4.49 (t, J=5.6 Hz, 1H), 4.45-4.33 (m, 1H), 3.30-3.26 (m, 3H), 2.15-2.10 (m, 2H), 1.92-1.84 (m, 4H), 1.45 (s, 9H), 1.18-1.09 (m, 2H); LC-MS (ESI⁺) m/z 364.2 (M+H)⁺.

### Step 2—Tert-butyl N-[6-fluoro-2-(4-formylcyclohexyl)indazol-5-yl]carbamate

To a solution of tert-butyl N-[6-fluoro-2-[4-(hydroxymethyl)cyclohexyl]indazol-5-yl]carbamate (100 mg, 275 umol) in DCM (1 mL) was added DMP (140 mg, 330 umol), then the reaction mixture was stirred at 25° C. for 2 hrs. On completion, the reaction mixture was quenched with Na₂S₂O₃ (5 mL) and NaHCO₃ (5 mL) and extracted with DCM (2×20 mL). The combined organic phase was washed with NaHCO₃ (10 mL) and brine (10 mL), dried with anhydrous Na₂SO₄, filtered and the filtrate was concentrated in vacuo to give the title compound (99 mg, 99% yield) as a purple solid. LC-MS (ESI⁺) m/z 362.4 (M+H)⁺.

### 3-[4-[1-[[4-(5-Amino-6-fluoro-indazol-2-yl)cyclohexyl]methyl]-4-piperidyl]-3-methyl-2-oxo-benzimidazol-1-yl]piperidine-2,6-dione (Intermediate BVE)

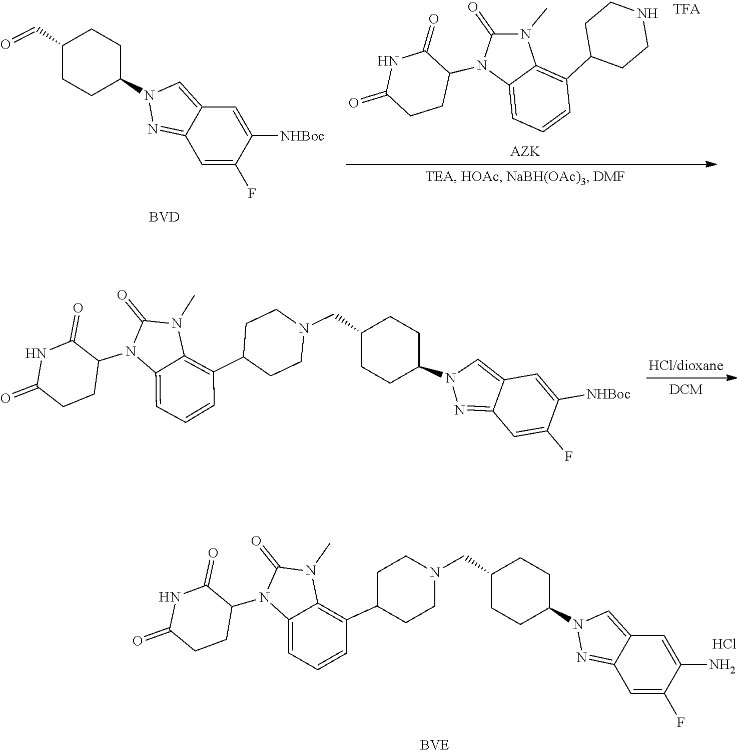

### Step 1—4-Tert-butyl N-[2-[4-[[4-[1-(2,6-dioxo-3-piperidyl)-3-methyl-2-oxo-benzimidazol-4-yl]-1-piperidyl]methyl]cyclohexyl]-6-fluoro-indazol-5-yl]carbamate

To a solution of 3-[3-methyl-2-oxo-4-(4-piperidyl)benzimidazol-1-yl]piperidine-2,6-dione (183 mg, 401 umol, TFA, Intermediate AZK) in DMF (2 mL) and THF (2 mL) was added TEA (40.6 mg, 401 umol, 55.8 uL), then the mixture was stirred at −10° C. for 10 mins. Next, tert-butyl N-[6-fluoro-2-(4-formylcyclohexyl)indazol-5-yl]carbamate (145 mg, 401 umol, Intermediate BVD) and NaBH(OAc)₃ (102 mg, 481 umol) was added to the mixture, and the mixture was stirred at −10° C. for 20 mins. Finally, AcOH (24.0 mg, 401 umol, 22.9 uL) was added to the mixture, and the mixture was stirred at −10° C. for 1 hr. On completion, the reaction mixture was quenched with water (0.5 mL) and concentrated in vacuo to give a residue. The residue was purified by reverse phase (0.1% FA condition) to give the title compound (200 mg, 72% yield) as a white solid. ¹H NMR (400 MHz, DMSO-d₆) δ 11.10 (s, 1H), 8.80-8.65 (m, 1H), 8.36 (s, 1H), 8.13 (s, 1H), 7.40-7.30 (m, 1H), 7.07-6.98 (m, 3H), 5.38 (dd, J=4.4, 12.0 Hz, 1H), 4.51-4.38 (m, 1H), 3.61 (s, 3H), 3.46-3.40 (m, 1H), 2.94-2.85 (m, 1H), 2.76-2.62 (m, 4H), 2.53-2.51 (m, 2H), 2.19-2.13 (m, 2H), 2.03-1.86 (m, 11H), 1.84-1.77 (m, 1H), 1.46 (s, 9H), 1.27-1.20 (m, 2H), LC-MS (ESI⁺) m/z 688.3 (M+H)⁺.

### Step 2—3-[4-[1-[[4-(5-Amino-6-fluoro-indazol-2-yl)cyclohexyl]methyl]-4-piperidyl]-3-methyl-2-oxo-benzimidazol-1-yl]piperidine-2,6-dione

To a solution of tert-butyl N-[2-[4-[[4-[1-(2,6-dioxo-3-piperidyl)-3-methyl-2-oxo-benzimidazol-4-yl]-1-piperidyl]methyl]cyclohexyl]-6-fluoro-indazol-5-yl]carbamate (85.0 mg, 123 umol) in DCM (1 mL) was added HCl/dioxane (4 M, 1 mL), then the reaction mixture was stirred at 25° C. for 0.5 hr. On completion, the reaction mixture was concentrated in vacuo to give the title compound (75 mg, 97% yield) as a white solid. LC-MS (ESI⁺) m/z 588.5 (M+H)⁺.

### 5-chloro-6-cyano-pyridine-3-carboxylic acid (Intermediate BVF)

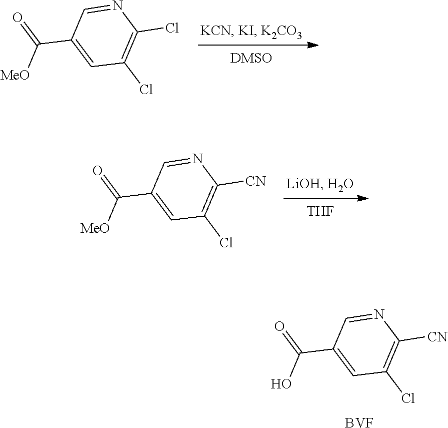

### Step 1—Methyl 5-chloro-6-cyano-pyridine-3-carboxylate

To a solution of methyl 5,6-dichloropyridine-3-carboxylate (1.00 g, 4.85 mmol, CAS #56055-54-0) in DMSO (10 mL) was added KCN (370 mg, 5.68 mmol) and KI (403 mg, 2.43 mmol) and K₂CO₃ (1.34 g, 9.71 mmol). The mixture was stirred at 50° C. for 3 hrs. On completion, the mixture was diluted with EA (50 mL) and water (30 mL). The organic phase was washed with brine (20 mL×3), dried with anhydrous Na₂SO₄, filtered and the filtrate was concentrated in vacuo. The residue was purified by silica gel chromatography (petroleum ether/ethyl acetate=8/1) to give the title compound (240 mg, 25% yield) as a white solid. ¹H NMR (400 MHz, DMSO-d₆) δ 9.12 (d, J=1.6 Hz, 1H), 8.68-8.64 (m, 1H), 3.94 (s, 3H).

### Step 2—5-chloro-6-cyano-pyridine-3-carboxylic acid

To a solution of methyl 5-chloro-6-cyano-pyridine-3-carboxylate (50.0 mg, 254 umol) in H₂O (0.5 mL) and THF (0.5 mL) was added LiOH·H₂O (25.0 mg, 595 umol). The mixture was stirred at 20° C. for 2 hrs. On completion, the mixture was acidified with KHSO₄ (aq) until the pH=5, then the mixture was extracted with ethyl acetate (20 mL×2). The combined organic layers were dried with anhydrous Na₂SO₄, filtered and the filtrate was concentrated in vacuo to give the title compound (20 mg, 430% yield) as a white solid. ¹H NMR (400 MHz, DMSO-d₆) δ 9.10 (d, J=1.6 Hz, 1H), 8.61 (d, J=1.8 Hz, 1H).

### 4-[5-[[6-(Trifluoromethyl)pyridine-2-carbonyl]amino]indazol-2-yl]cyclohexanecarboxylic acid (Intermediate BVG)

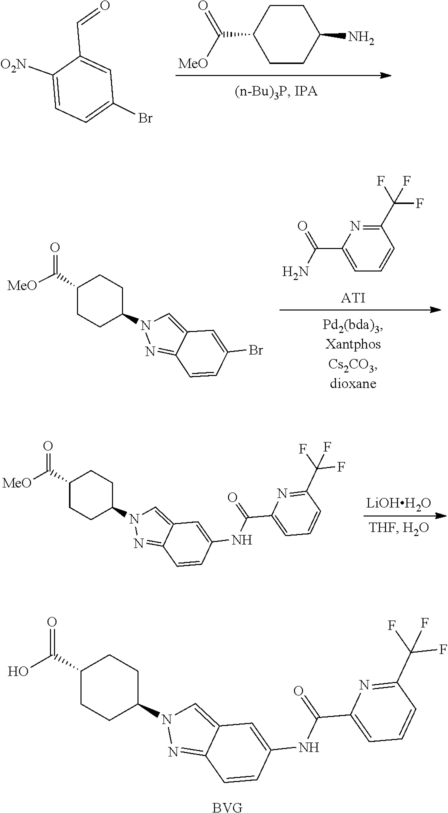

### Step 1—Methyl 4-(5-bromoindazol-2-yl)cyclohexanecarboxylate

To a mixture of methyl 4-aminocyclohexanecarboxylate (4.51 g, 28.6 mmol CAS #62456-15-9) in IPA (100 mL) was added 5-bromo-2-nitro-benzaldehyde (6.00 g, 26.0 mmol CAS #20357-20-4). The mixture was stirred at 25° C. for 3 hours. Next, tributylphosphane (15.8 g, 78.2 mmol, 19.3 mL) was added and the reaction mixture was stirred at 80° C. for 4 hours. On completion, the reaction mixture was concentrated in vacuo. The residue was purified by column chromatography to give the title compound (3.80 g, 43% yield) as white solid. ¹H NMR (400 MHz, DMSO-d₆) δ 8.38 (s, 1H), 7.95 (s, 1H), 7.58 (d, J=9.2 Hz, 1H), 7.30 (dd, J=1.6, 9.2 Hz, 1H), 4.53 (tt, J=3.6, 11.6 Hz, 1H), 3.63 (s, 3H), 2.49-2.41 (m, 1H), 2.15 (d J=9.6 Hz, 2H), 2.07 (d, J=13.2 Hz, 2H), 2.02-1.88 (m, 2H), 1.68-1.52 (m, 2H).

### Step 2—Methyl 4-[5-[[6-(trifluoromethyl)pyridine-2-carbonyl]amino]indazol-2-yl]cyclohexanecarboxylate

To a mixture of methyl 4-(5-bromoindazol-2-yl)cyclohexanecarboxylate (2.00 g, 5.93 mmol) and 6-(trifluoromethyl)pyridine-2-carboxamide (1.35 g, 7.12 mmol, Intermediate ATI) in dioxane (20 mL) was added in Cs₂CO₃ (3.86 g, 11.8 mmol), Xantphos (686 mg, 1.19 mmol) and Pd₂(dba)₃ (543 mg, 593 umol), then the reaction mixture was stirred at 80° C. for 12 hours. On completion, the residue was diluted with water (30 mL) and extracted with EA (3×100 mL). The combined organic layer was dried over Na₂SO₄, filtered and concentrated in vacuo to give a residue. The residue was purified by reverse phase (0.1% FA condition) and then the residue was purified by column chromatography to give the title compound (770 mg, 29% yield) as yellow solid. ¹H NMR (400 MHz, DMSO-d₆) δ 10.36 (s, 1H), 8.42-8.33 (m, 3H), 8.29 (s, 1H), 8.17 (d, J=7.6 Hz, 1H), 7.63-7.53 (m, 2H), 4.54-4.46 (m, 1H), 3.63 (s, 3H), 2.48-2.43 (m, 1H), 2.18 (d, J=9.6 Hz, 2H), 2.08 (d, J=11.6 Hz, 2H), 2.03-1.93 (m, 2H), 1.67-1.53 (m, 2H).

### Step 3—4-[5-[[6-(Trifluoromethyl)pyridine-2-carbonyl]amino]indazol-2-yl]cyclohexanecarboxylic acid

To a mixture of methyl 4-[5-[[6-(trifluoromethyl) pyridine-2-carbonyl]amino]indazol-2-yl]cyclohexanecarboxylate (300 mg, 672 umol) in MeOH (0.5 mL), H₂O (2 mL) and THF (6 mL) was added LiOH·H₂O (84.5 mg, 2.02 mmol). The reaction mixture was stirred at 25° C. for 1.5 hours. On completion, the residue was acidified with HCl (1N) until the pH=4-5. Then the residue mixture was filtered, the filter cake was dried to give the title compound (280 mg, 96% yield) as yellow solid. LC-MS (ESI+) m/z 433.2 (M+H)⁺.

### 6-Cyano-N-[2-(4-formylcyclohexyl)indazol-5-yl]pyridine-2-carboxamide (Intermediate BVH)

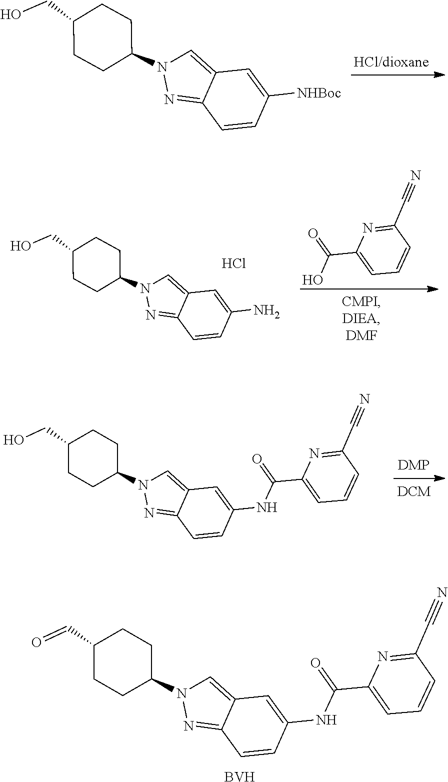

### Step 1—6-Cyano-N-[2-[4-(hydroxymethyl)cyclohexyl]indazol-5-yl]pyridine-2-carboxamide

To a solution of 6-cyanopyridine-2-carboxylic acid (126 mg, 851 umol, CAS #872602-74-9) in DMF (3 mL) was added DIEA (330 mg, 2.56 mmol, 445 uL) and CMPI (326 mg, 1.28 mmol). Then [4-(5-aminoindazol-2-yl)cyclohexyl]methanol (240 mg, 851 umol, HCl, synthesized via Step 1 of Intermediate BUT) was added and the mixture was stirred at 25° C. for 2 hrs. On completion, the reaction mixture was concentrated in vacuo to give a residue. The residue was purified by column chromatography to give the title compound (130 mg, 40% yield) as a white solid. ¹HNMR (400 MHz, DMSO-d₆) δ 10.59 (s, 1H), 8.44-8.37 (m, 2H), 8.34-8.27 (m, 3H), 7.60 (d, J=0.8 Hz, 2H), 4.48-4.36 (m, 1H), 3.31-3.28 (m, 3H), 2.20 -2.10 (m, 2H), 1.98-1.83 (m, 4H), 1.56-1.42 (m, 1H), 1.22-1.09 (m, 2H); LC-MS (ESI⁺) m/z 376.1 (M+H)⁺.

### Step 2—6-Cyano-N-[2-(4-formylcyclohexyl)indazol-5-yl]pyridine-2-carboxamide

To a solution of 6-cyano-N-[2-[4-(hydroxymethyl)cyclohexyl]indazol-5-yl]pyridine-2-carboxamide (65.0 mg, 173 umol) in DCM (2 mL) was added DMP (88.1 mg, 207 umol, 64.3 uL). The reaction mixture was stirred at 25° C. for 2 hrs. On completion, the reaction mixture was quenched with Na₂S₂O₃ (5 mL) and NaHCO₃ (5 mL) and extracted with DCM (2×10 mL). The combined organic phase was washed with NaHCO₃ (10 mL) and brine (2×10 mL), dried with anhydrous Na₂SO₄, filtered and the filtrate was concentrated in vacuo to give the title compound (64.0 mg, 98% yield) as a white solid. LC-MS (ESI⁺) m/z 374.1 (M+H)⁺.

### 2-[4-[[Tert-butyl(dimethyl)silyl]oxymethyl]cyclohexyl]indazol-5-amine (Intermediate BVI)

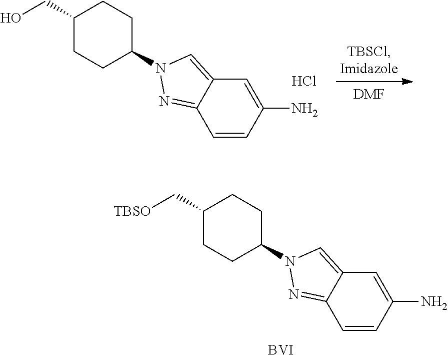

To a solution of [4-(5-aminoindazol-2-yl)cyclohexyl]methanol (240 mg, 851 umol, HCl, synthesized via Step 1 of Intermediate BUT) in THF (5 mL) was added imidazole (115 mg, 1.70 mmol) and TBSCI (166 mg, 1.11 mmol, 135 uL), then the reaction mixture was stirred at 25° C. for 2 hrs. On completion, the residue was diluted with water (10 mL) and extracted with DCM (2×15 mL). The combined organic layer was washed with brine (2×10 mL) and dried over Na₂SO₄, filtered and the filtrate was concentrated in vacuo to give the title compound (300 mg, 97% yield) as a brown solid. LC-MS (ESI⁺) m/z 360.2 (M+H)⁺.

### 5-Cyano-N-[2-(4-formylcyclohexyl)indazol-5-yl]pyrrolo[2,3-b]pyridine-1-carboxamide amine (Intermediate BVJ)

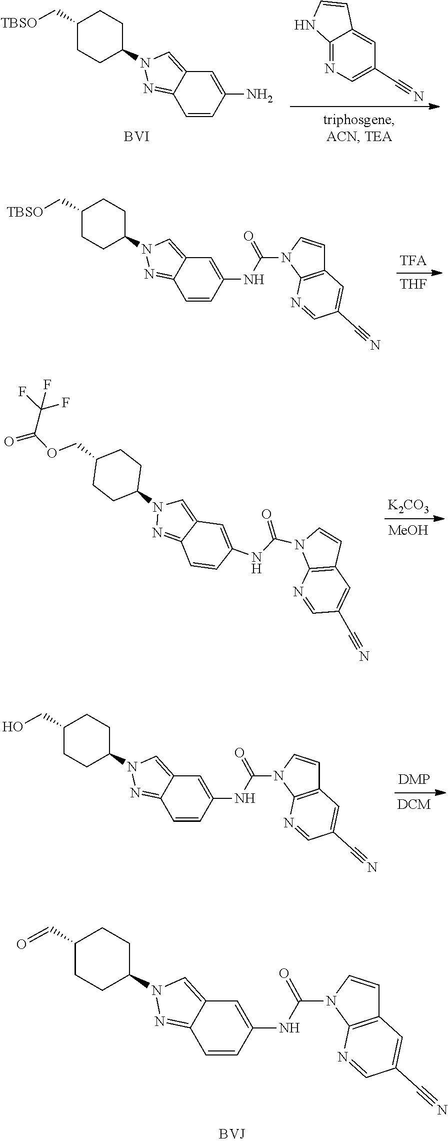

### Step 1—N-[2-[4-[[tert-butyl(dimethyl)silyl]oxymethyl]cyclohexyl]indazol-5-yl]-5-cyano-pyrrolo [2,3-b]pyridine-1-carboxamide

To a solution of 1H-pyrrolo[2,3-b]pyridine-5-carbonitrile (119 mg, 834 umol, CAS #517918-95-5) in ACN (3 mL) was added TEA (422 mg, 4.17 mmol, 580 uL) and the reaction mixture was stirred at 0° C., then triphosgene (148 mg, 500 umol) was added and the reaction mixture was stirred at 0° C. for 0.5 hr. Next, 2-[4-[[tert-butyl(dimethyl)silyl]oxymethyl]cyclohexyl]indazol-5-amine (300 mg, 834 umol, Intermediate BVI) was added, and the reaction mixture was stirred at 80° C. for 0.5 hr. On completion, the mixture was quenched with NaHCO₃ (5 mL) and diluted with water (10 mL) and extracted with DCM (2×15 mL). The combined organic layers were washed with brine (10 mL), dried over Na₂SO₄, filtered and the filtrate was concentrated in vacuo to give a residue. The residue was purified by reverse phase (0.1% FA condition) to give the title compound (250 mg, 56% yield) as a white solid. ¹H NMR (400 MHz, DMSO-d₆) δ 11.39 (s, 1H), 8.95 (d, J=2.0 Hz, 1H), 8.77 (d, J=2.0 Hz, 1H), 8.43 (s, 1H), 8.25 (d, J=4.0 Hz, 1H), 8.11 (d, J=2.0 Hz, 1H), 7.68 (d, J=9.2 Hz, 1H), 7.38 (dd, J=2.0, 9.2 Hz, 1H), 6.92 (d, J=4.0 Hz, 1H), 4.49-4.38 (m, 1H), 3.48 (d, J=6.0 Hz, 2H), 2.20-2.15 (m, 2H), 1.95-1.85 (m, 4H), 1.60-1.53 (m, 1H), 1.20-1.14 (m, 2H), 0.89 (s, 9H), 0.06 (s, 6H), LC-MS (ESI⁺) m/z 529.4 (M+H)⁺.

### Step 2—[4-[5-[(5-Cyanopyrrolo[2,3-b]pyridine-1-carbonyl)amino]indazol-2-yl]cyclohexyl]methyl 2,2,2-trifluoroacetate

To a solution of N-[2-[4-[[tert-butyl(dimethyl)silyl]oxymethyl]cyclohexyl]indazol-5-yl]-5-cyano-pyrrolo[2,3-b]pyridine-1-carboxamide (90.0 mg, 170 umol) in DCM (2 mL) was added TFA (116 mg, 1.02 mmol, 75.6 uL). The reaction mixture was stirred at 25° C. for 0.5 hr. On completion, the reaction mixture was diluted with THF (3 mL), then the mixture was extracted with DCM (2×5 mL) and washed with (2×5 mL). The combined organic layers was dried over Na₂SO₄, filtered and the filtrate was concentrated in vacuo to give the title compound (86.0 mg, 98% yield) as a white solid. LCMS (ESI⁺) m/z 511.1 (M+H)⁺.

### Step 3—5-Cyano-N-[2-[4-(hydroxymethyl)cyclohexyl]indazol-5-yl]pyrrolo[2,3-b]pyridine-1-carboxamide

To a solution of [4-[5-[(5-cyanopyrrolo[2,3-b]pyridine-1-carbonyl)amino]indazol-2-yl]cyclohexyl]methyl 2,2,2-trifluoroacetate (50.0 mg, 97.9 umol) in MeOH (1 mL) was added K₂CO₃ (27.0 mg, 195 umol), then the reaction mixture was stirred at 25° C. for 2 hrs. On completion, the reaction mixture was filtered and the filtrate was diluted with water (3 mL) and extracted with DCM (2×10 mL). The combined organic layers was washed with brine (2×15 mL) and dried over Na₂SO₄, filtered and the filtrate was concentrated in vacuo to give the title compound (40.0 mg, 98% yield) as a brown solid. LCMS (ESI⁺) m/z 415.2 (M+H)⁺.

### Step 4—5-Cyano-N-[2-(4-formylcyclohexyl)indazol-5-yl]pyrrolo[2,3-b]pyridine-1-carboxamide

To a solution of 5-cyano-N-[2-[4-(hydroxymethyl)cyclohexyl]indazol-5-yl]pyrrolo[2,3-b]pyridine-1-carboxamide (40.0 mg, 96.5 umol) in DCM (2 mL) was added DMP (49.1 mg, 115 umol, 35.8 uL), then the reaction mixture was stirred at 25° C. for 2 hrs. On completion, the reaction mixture was quenched with Na₂S₂O₃ (3 mL) and NaHCO₃ (3 mL), then extracted with DCM (2×8 mL). The combined organic phase was washed with NaHCO₃ (5 mL) and brine (2×8 mL), dried with anhydrous Na₂SO₄, filtered and the filtrate was concentrated in vacuo to give the title compound (39.0 mg, 97% yield) as a brown solid. LCMS (ESI⁺) m/z 413.2 (M+H)⁺.

### 3-[5-Fluoro-3-methyl-2-oxo-4-(4-piperidyl)benzimidazol-1-yl]piperidine-2,6-dione (Intermediate BVK)

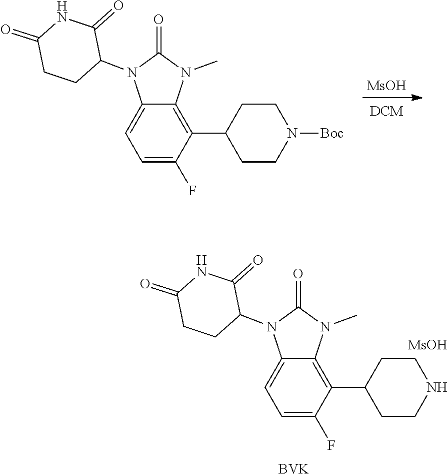

To a mixture of tert-butyl 4-[1-(2,6-dioxo-3-piperidyl)-5-fluoro-3-methyl-2-oxo-benzimidazol-4-yl]piperidine-1-carboxylate (60.0 mg, 130 umol, synthesized via Steps 1-3 of Intermediate BSP) in THF (1 mL) was added MsOH (37.5 mg, 390 umol, 27.8 uL). The reaction mixture was stirred at 65° C. for 1 hour. On completion, the reaction mixture was triturated with MTBE (5 mL). White solid formed which was filtered to give the title compound (46.0 mg, 97% yield) as white solid. LC-MS (ESI⁺) m/z 361.1 (M+H)⁺.

### 6-(5-cyanopyrrolo[2,3-b]pyridin-1-yl)-N-[(4-formylcyclohexyl)methyl]-4-(isopropylamino) pyridine-3-carboxamide (Intermediate BVL)

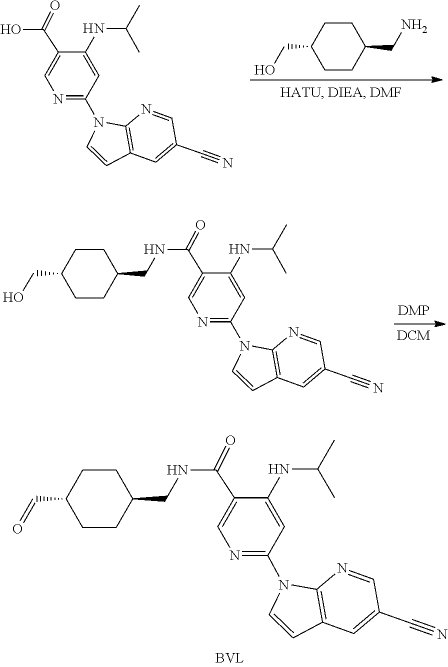

### Step 1—6-(5-cyanopyrrolo[2,3-b]pyridin-1-yl)-N-[[4-(hydroxymethyl)cyclohexyl]methyl]-4-(isopropylamino)pyridine-3-carboxamide

To a solution of 6-(5-cyanopyrrolo[2,3-b]pyridin-1-yl)-4-(isopropylamino)pyridine-3-carboxylic acid (200 mg, 622 umol, Intermediate BUQ) and [4-(aminomethyl)cyclohexyl]methanol (115 mg, 809 umol, CAS #17879-23-1) in DMF (4 mL) were added DIEA (160 mg, 1.24 mmol) and HATU (473 mg, 1.24 mmol) at 25° C. The reaction mixture was stirred at 25° C. for 1 hr. On completion, the reaction mixture was diluted with EtOAc (20 ml). The organic layer was washed with water (20 mL×3) and concentrated in vacuo. The residue was purified by column chromatography (SiO₂, petroleum ether/ethyl acetate=1/1, P1:R_{f}=0.3) to give title compound (260 mg, 93% yield) as pink solid. ¹H NMR (400 MHz, DMSO-d₆) δ 8.80 (d, J=2.0 Hz, 1H), 8.66 (d, J=2.0 Hz, 1H), 8.62 (d, J=7.2 Hz, 1H), 8.57 (s, 1H), 8.56 -8.53 (m, 1H), 8.52 (d, J=4.0 Hz, 1H), 8.07 (s, 1H), 6.88 (d, J=4.0 Hz, 1H), 4.34 (t, J=5.2 Hz, 1H), 3.83-3.70 (m, 1H), 3.20 (t, J=5.6 Hz, 2H), 3.09 (t, J=6.4 Hz, 2H), 2.69 (s, 3H), 1.81-1.71 (m, 4H), 1.55-1.44 (m, 1H), 1.29 (d, J=6.4 Hz, 6H).

### Step 2—6-(5-cyanopyrrolo[2,3-b]pyridin-1-yl)-N-[(4-formylcyclohexyl)methyl]-4-(isopropylamino) pyridine-3-carboxamide

To a solution of 6-(5-cyanopyrrolo[2,3-b]pyridin-1-yl)-N-[[4-(hydroxymethyl)cyclohexyl]methyl]-4-(isopropylamino)pyridine-3-carboxamide (210 mg, 470 umol) in DCM (4 mL) and DMF (0.1 mL) was added DMP (398 mg, 940 umol). The reaction was stirred at 25° C. for 1 hr. On completion, the reaction mixture was quenched with Na₂S₂O₃ (6 mL) and NaHCO₃ (6 mL) aqueous. The combined layers were extracted with DCM (30 mL×2). The combined organic layers were concentrated in vacuo to give title compound (155 mg, 348 umol, 74% yield) as yellow solid. LC-MS (ESI⁺) m/z 445.2 (M+H)⁺.

### 3-[(4-methoxyphenyl)methyl]hexahydropyrimidine-2,4-dione (Intermediate BTJ)

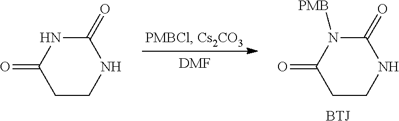

To the solution of hexahydropyrimidine-2,4-dione (3.0 g, 26.3 mmol, CAS #504-07-4) in DMF (60 mL) was added Cs₂CO₃ (17.1 g, 52.6 mmol) at 25° C., then 1-(chloromethyl)-4-methoxybenzene (3.71 g, 23.6 mmol) was dropwise added to the mixture slowly at 25° C. The mixture was stirred at 25° C. for 2 hours. On completion, the reaction was filtered and the filter cake was washed by EA (30 mL×2). The filtrate was poured into water (150 mL) and extracted with EA (100 mL×2). The combined organic layer was washed with water (100 mL) and saturated brine (100 mL). The organic layer was dried over Na₂SO₄, filtered and concentrated to give the crude product. The crude product was suspended in EA/PE (1/1, 80 mL) and stirred for 0.5 hour. The suspension was filtered, the filter cake was dried to give compound (2.80 g, 45% yield) as a white solid. ¹H NMR (400 MHz, CHLOROFORM-d) δ 7.42-7.30 (m, 2H), 6.90-6.62 (m, 2H), 6.15 (s, 1H), 4.88 (s, 2H), 3.78 (s, 3H), 3.37 (dt, J=2.4, 6.8 Hz, 2H), 2.71 (t, J=6.8 Hz, 2H).

### 1-(7-Bromoimidazo[1,2-a]pyridin-3-yl)hexahydropyrimidine-2,4-dione (Intermediate BTK)

### Step 1—7-bromo-3-iodo-imidazo[1,2-a]pyridine

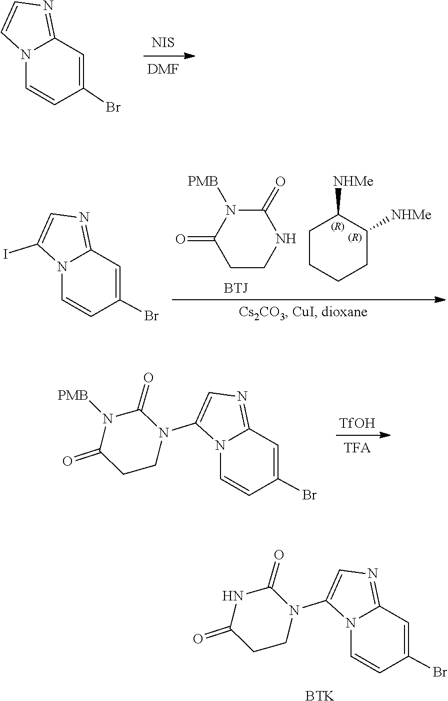

To a solution of 7-bromoimidazo[1,2-a]pyridine (9.50 g, 48.2 mmol, CAS #808744-34-5) in DMF (150 mL) was added NIS (13.0 g, 57.8 mmol) at 25° C. The mixture was stirred at 100° C. for 1 hour. On completion, the reaction mixture was poured into 400 mL of water and extracted with EtOAc (200 mL×2). The organic layer was washed with water (200 mL) and saturated brine (200 mL), then dried over Na₂SO₄, filtered and concentrated to give the crude product. The crude product was purified by flash silica gel chromatography (120 g Column, Eluent of 0-5% ethyl acetate/petroleum ether gradient @150 mL/min) to give the compound (11.6 g, 74% yield) as a black brown solid. ¹H NMR (400 MHz, CHLOROFORM-d) δ 8.00 (d, J=7.2 Hz, 1H), 7.82 (d, J=1.2 Hz, 1H), 7.67 (s, 1H), 7.04 (dd, J=2.0, 7.3 Hz, 1H).

### Step 2—1-(7-bromoimidazo[1,2-a]pyridin-3-yl)-3-[(4-methoxyphenyl)methyl]hexahydropyrimidine-2,4-dione

To a solution of 3-[(4-methoxyphenyl)methyl]hexahydropyrimidine-2,4-dione (4 g, 17.08 mmol, Intermediate BTJ), 7-bromo-3-iodo-imidazo[1,2-a]pyridine (6.62 g, 20.49 mmol) in 1,4-dioxane (100 mL) was added Cs₂CO₃ (11.1 g, 34.1 mmol), CuI (650 mg, 3.42 mmol) and (1R,2R)-N1,N2-Dimethylcyclohexane-1,2-diamine (485 mg, 3.42 mmol, CAS #68737-65-5) at 25° C. under N₂. Then the mixture was stirred at 80° C. for 16 hours. On completion, the reaction mixture was poured into 200 mL of water and extracted with EtOAc (100 mL×2). The combined organic layers were washed with water (200 mL) and saturated brine (200 mL), then dried over Na₂SO₄, filtered and concentrated to give a crude product. The crude product was purified by silica gel chromatography (eluted with petroleum ether/ethyl acetate=10/1 to 0/1_ to give the title compound (2.00 g, 27% yield) as a yellow solid.

### Step 3—1-(7-Bromoimidazo[1,2-a]pyridin-3-yl)hexahydropyrimidine-2,4-dione

A solution of 1-(7-bromoimidazo[1,2-a]pyridin-3-yl)-3-[(4-methoxyphenyl)methyl]hexahydropyrimidine-2,4-dione (2.30 g, 5.36 mmol) in TfOH (1.5 mL) was stirred at 65° C. for 4 hours. On completion, the mixture was concentrated to give residue, then the residue was adjusted pH to 6-7 with TEA at 0° C. Then the mixture was concentrated to give a residue. The residue was suspended in EtOAc (30 mL) and stirred for 0.5 hour. Next, the suspension was filtered and the filter cake was concentrated to give the title compound (1.55 g, 84% yield) as a white solid. ¹H NMR (400 MHz, DMSO-d₆) δ 10.69 (s, 1H), 8.32 (d, J=7.2 Hz, 1H), 7.93 (d, J=1.2 Hz, 1H), 7.59 (s, 1H), 7.15 (dd, J=2.0, 7.2 Hz, 1H), 3.81 (t, J=6.8 Hz, 2H), 2.83 (t, J=6.4 Hz, 2H).

### 4-Bromo-3-methyl-1-(2-trimethylsilylethoxymethyl)benzimidazol-2-one (Intermediate BED)

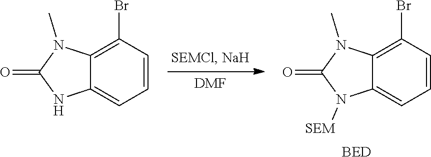

To a mixture of 4-bromo-3-methyl-1H-benzimidazol-2-one (50.0 g, 220 mmol, synthesized via Steps 1-3 of Intermediate HP) in DMF (500 mL) was added NaH (13.2 g, 330 mmol, 60% dispersion in mineral oil) at 0° C. The reaction mixture was stirred for 30 minutes. Then SEMCl (44.0 g, 264 mmol) was added dropwise at 0° C. and the reaction mixture was stirred for 16 hours. On completion, the mixture was poured into water (500 mL). The mixture was extracted with DCM (3×200 mL) and the combined organic layer was dried over by Na₂SO₄. The mixture was filtrated and the filtrate was concentrated in vacuo. The residue was purified by column chromatography (petroleum ether/ethyl acetate=20/1) to give the title compound (60.0 g, 76% yield) as yellow oil. ¹H NMR (400 MHz, DMSO-d₆) δ 7.34-7.21 (m, 2H), 7.01-6.97 (m, 1H), 5.24 (s, 2H), 3.61 (s, 3H), 3.55-3.51 (m, 2H), 0.85-0.81 (m, 2H), 0.07 (s, 9H).

### 3-(4-((1R,4R)-2,5-diazabicyclo[2.2.1]heptan-2-yl)-3-methyl-2-oxo-2,3-dihydro-1H-benzo[d]imidazol-1-yl)piperidine-2,6-dione (Intermediate BDY)

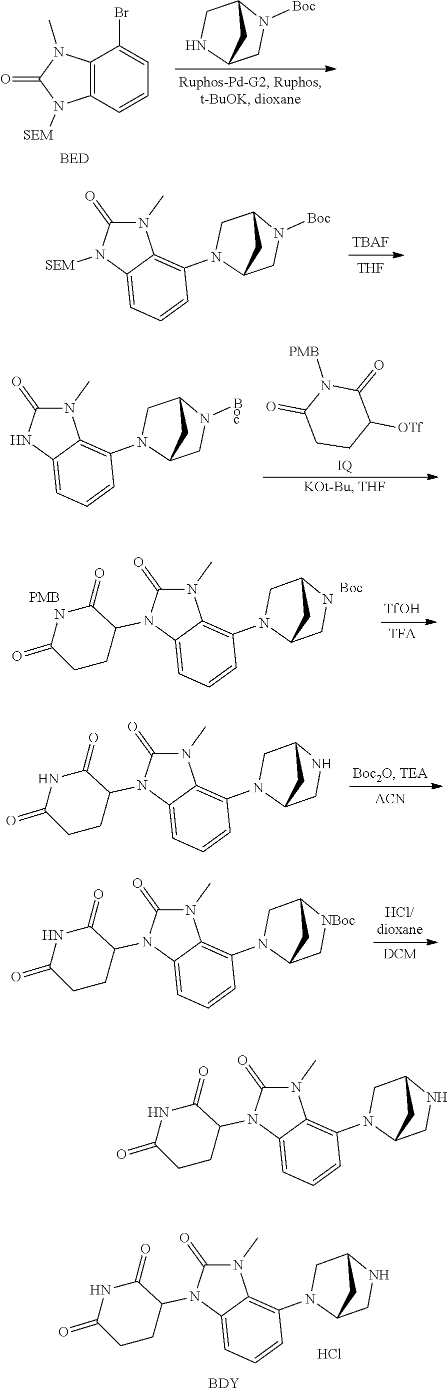

### Step 1—(1R,4R)-tert-butyl 5-(3-methyl-2-oxo-1-((2-(trimethylsilyl)ethoxy)methyl)-2,3-dihydro-1H-benzo[d]imidazol-4-yl)-2,5-diazabicyclo[2.2.1]heptane-2-carboxylate

To a solution of 4-bromo-3-methyl-1-(2-trimethylsilylethoxymethyl)benzimidazol-2-one (3.00 g, 8.40 mmol, Intermediate BED), tert-butyl (1R,4R)-2,5-diazabicyclo[2.2.1]heptane-2-carboxylate (1.66 g, 8.40 mmol, CAS #134003-84-2) and t-BuOK (1.88 g, 16.8 mmol) in dioxane (40 mL) was added RuPhos (196 mg, 420 umol) and [2-(2-aminophenyl)phenyl]-chloro-palladium; dicyclohexyl-[2-(2,6-diisopropoxyphenyl)phenyl]phosphane (326 mg, 420 umol). The reaction mixture was degassed and purged with nitrogen for 3 times, and then the mixture was stirred at 80° C. for 2 hrs under nitrogen atmosphere. On completion, the reaction mixture was filtered and the combined filtrate was concentrated in vacuo to give a residue. The residue was purified by silica gel chromatography (petroleum ether:ethyl acetate=3:1) to give the title compound (2.86 g, 710% yield) as yellow oil. ¹H NMR (400 MHz, DMSO-d₆) δ 7.05-6.90 (m, 2H), 6.81 (d, J=8.0 Hz, 1H), 5.21 (s, 2H), 4.37 (m, 1H), 3.90 (m, 1H), 3.58 (s, 3H), 3.53 (t, J=8.0 Hz, 2H), 3.44-3.35 (m, 1H), 3.31-3.16 (m, 2H), 3.15-3.06 (m, 1H), 2.04 (m, 1H), 1.88 (m, 1H), 1.43 (m, 9H), 0.88-0.76 (m, 2H), −0.03-−0.13 (m, 9H). LC-MS (ESI⁺) m/z 475.3 (M+H)⁺.

### Step 2—(1R,4R)-tert-butyl 5-(3-methyl-2-oxo-2,3-dihydro-1H-benzo[d]imidazol-4-yl)-2,5-diazabicyclo[2.2.1]heptanes-2-carboxylate

A solution of tert-butyl (1R,4R)-5-[3-methyl-2-oxo-1-(2-trimethylsilylethoxymethyl)benzimidazol-4-yl]-2,5-diazabicyclo[2.2.1]heptane-2-carboxylate (2.76 g, 5.81 mmol) in TBAF (40 mL, 1 M) was stirred at 80° C. for 16 hrs. On completion, the reaction mixture was diluted with water (80 mL) and extracted with ethyl acetate (3×25 mL). The combined organic layers were washed with sodium chloride solution (50 mL), dried over anhydrous sodium sulfate, filtered and concentrated in vacuo to give a residue. The residue was purified by silica gel chromatography (petroleum ether:ethyl acetate=1:2) to give the title compound (1.60 g, 79% yield) as a yellow solid. 1H NMR (400 MHz, CDCl₃) δ 10.34 (m, 1H), 7.03-6.94 (m, 1H), 6.88 (m, 1H), 6.81 (d, J=8.0 Hz, 1H), 4.66-4.41 (m, 1H), 3.85 (m, 1H), 3.73 (s, 3H), 3.66-3.53 (m, 1H), 3.48 (m, 1H), 3.30-3.12 (m, 2H), 2.17-2.07 (m, 1H), 1.95 (m, 1H), 1.51 (m, 9H). LC-MS (ESI⁺) m/z 345.4 (M+H)⁺.

### Step 3—(1R,4R)-tert-butyl 5-(1-(1-(4-methoxybenzyl)-2,6-dioxopiperidin-3-yl)-3-methyl-2-oxo-2,3-dihydro-1H-benzo[d]imidazol-4-yl)-2,5-diazabicyclo[2.2.1]heptane-2-carboxylate

To a solution of tert-butyl (1R,4R)-5-(3-methyl-2-oxo-1H-benzimidazol-4-yl)-2,5-diazabicyclo[2.2.1]heptanes-2-carboxylate (1.30 g, 3.77 mmol) in THF (30 mL) was added tBuOK (635 mg, 5.66 mmol) at 0° C. and stirred for 0.5 hr. Then the mixture was added [1-[(4-methoxyphenyl)methyl]-2,6-dioxo-3-piperidyl]trifluoromethanesulfonate (2.16 g, 5.66 mmol, Intermediate IQ) in batch at 0° C. and stirred at 25° C. for 2 hrs. On completion, the reaction mixture was poured into saturated ammonium chloride aqueous solution (50 mL) at 0° C. and extracted with ethyl acetate (3×25 mL). The combined organic layers were washed with sodium chloride solution (25 mL), dried over anhydrous sodium sulfate, filtered and concentrated in vacuo to give a residue. The residue was purified by silica gel chromatography (petroleum ether:ethyl acetate=1:1) and reversed-phase HPLC (FA condition) to give the title compound (1.50 g, 69% yield) as a blue solid. ¹H NMR (400 MHz, CDCl₃) δ 7.37 (dd, J=8.8, 2.0 Hz, 2H), 6.90-6.77 (m, 4H), 6.27 (m, 1H), 5.34-5.08 (m, 1H), 5.03-4.87 (m, 2H), 4.61-4.43 (m, 1H), 3.86-3.76 (m, 4H), 3.72 (s, 3H), 3.63-3.40 (m, 2H), 3.29-3.13 (m, 2H), 3.05-2.95 (m, 1H), 2.89-2.76 (m, 1H), 2.68-2.51 (m, 1H), 2.21-2.07 (m, 3H), 1.96 (m, 1H), 1.51 (m, 9H). LC-MS (ESI⁺) m/z 576.2 (M+H)⁺.

### Step 4—3-(4-((1R,4R)-2,5-diazabicyclo[2.2.1]heptan-2-yl)-3-methyl-2-oxo-2,3-dihydro-1H-benzo[d]imidazol-1-yl)piperidine-2,6-dione

A solution of tert-butyl (1R,4R)-5-[1-[1-[(4-methoxyphenyl)methyl]-2,6-dioxo-3-piperidyl]-3-methyl-2-oxo-benzimidazol-4-yl]-2,5-diazabicyclo[2.2.1]heptane-2-carboxylate (800 mg, 1.39 mmol) in TfOH (1 mL) and TFA (10 mL) was stirred at 60° C. for 16 hrs. On completion, the reaction mixture was concentrated in vacuo to give the title compound (652 mg, 70% yield) as green oil. LC-MS (ESI⁺) m/z 356.1 (M+H)⁺.

### Step 5—(1R,4R)-tert-butyl 5-(1-(2,6-dioxopiperidin-3-yl)-3-methyl-2-oxo-2,3-dihydro-1H-benzo[d]imidazol-4-yl)-2,5-diazabicyclo[2.2.1]heptane-2-carboxylate

To a solution of 3-[4-[(1R,4R)-2,5-diazabicyclo[2.2.1]heptan-2-yl]-3-methyl-2-oxo-benzimidazol-1-yl]piperidine-2,6-dione (610 mg, 1.30 mmol, TFA) in ACN (5 mL) was added TEA (394 mg, 3.90 mmol) and (Boc)₂O (340 mg, 1.56 mmol). The mixture was stirred at 25° C. for 2 hrs. On completion, the reaction mixture was concentrated in vacuo to give a residue. The residue was diluted with saturated ammonium chloride aqueous solution (25 mL) and extracted with ethyl acetate (3×15 mL). The combined organic layers were washed with sodium chloride solution (25 mL), dried over anhydrous sodium sulfate, filtered and concentrated in vacuo to give a residue. The residue was purified by silica gel chromatography (petroleum ether:ethyl acetate=1:5) to give the title compound (580 mg, 90% yield) as a green solid. ¹H NMR (400 MHz, CDCl₃) δ 8.25 (m, 1H), 7.05-6.94 (m, 1H), 6.85 (d, J=8.0 Hz, 1H), 6.57 (d, J=7.6 Hz, 1H), 5.29-5.15 (m, 1H), 4.63-4.43 (m, 1H), 3.90-3.79 (m, 1H), 3.73 (s, 3H), 3.58 (m, 1H), 3.47 (m, 1H), 3.31-3.13 (m, 2H), 2.98-2.65 (m, 3H), 2.29-2.16 (m, 1H), 2.11 (m, 1H), 1.97 (m, 1H), 1.51 (m, 9H). LC-MS (ESI⁺) m/z 456.3 (M+H)⁺.

### Step 6—3-(4-((1R,4R)-2,5-diazabicyclo[2.2.1]heptan-2-yl)-3-methyl-2-oxo-2,3-dihydro-1H-benzo[d]imidazol-1-yl)piperidine-2,6-dione

A solution of tert-butyl (1R,4R)-5-[1-(2,6-dioxo-3-piperidyl)-3-methyl-2-oxo-benzimidazol-4-yl]-2,5-diazabicyclo[2.2.1]heptane-2-carboxylate (100 mg, 220 umol) in HCl/dioxane (1 mL) and DCM was stirred at 25° C. for 0.5 hr. On completion, the reaction mixture was concentrated in vacuo to give the title compound (86 mg, 99% yield) as a yellow solid. LC-MS (ESI⁺) m/z 356.4 (M+H)⁺.

### 3-(5-bromo-3-methyl-2-oxo-benzimidazol-1-yl)piperidine-2,6-dione (Intermediate HN)

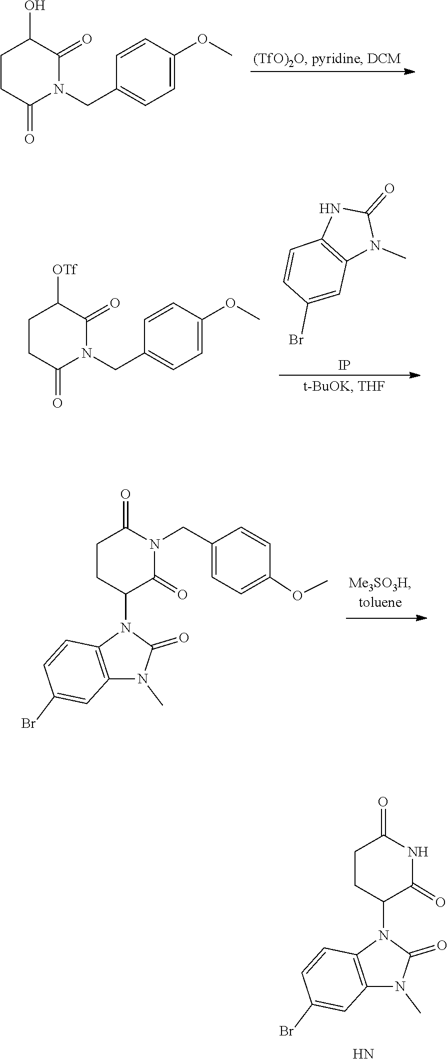

### Step 1—[1-[(4-Methoxyphenyl) methyl]-2,6-dioxo-3-piperidyl]trifluoromethanesulfonate

To a solution of 3-hydroxy-1-[(4-methoxyphenyl) methyl]piperidine-2,6-dione (43.0 g, 173 mmol, CAS #2357109-89-6) and pyridine (27.3 g, 345 mmol) in DCM (500 mL) was added trifluoromethylsulfonyl trifluoromethanesulfonate (73.0 g, 258.74 mmol) dropwise at 0° C. The mixture was stirred at 0-10° C. for 1.5 hours under N₂. On completion, the mixture was concentrated in vacuo. The residue was purified by column chromatography on silica gel to give the title compound (45.0 g, 68% yield) as light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### Step 2—3-(5-Bromo-3-methyl-2-oxo-benzimidazol-1-yl)-1-[(4-methoxyphenyl)methyl]piperidine-2,6-dione

To a solution of 5-bromo-3-methyl-1H-benzimidazol-2-one (4.90 g, 21.6 mmol, Intermediate IP) in THF (300 mL) was added t-BuOK (3.63 g, 32.3 mmol) at 0° C. The mixture was stirred at 0-10° C. for 1 hour under N₂. Then a solution of [1-[(4-methoxyphenyl) methyl]-2, 6-dioxo-3-piperidyl]trifluoromethanesulfonate (9.87 g, 25.9 mmol) in THF (100 mL) was added to the reaction mixture at 0-10° C. during 30 minutes. The mixture was stirred at 0-10° C. for 30 minutes under N₂. An additional solution of [1-[(4-methoxyphenyl) methyl]-2, 6-dioxo-3-piperidyl]trifluoromethanesulfonate (2.47 g, 6.47 mmol) in THF (20 mL) was added to the reaction mixture at 0-10° C. dropwise. The mixture was then stirred at 0-10° C. for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. ¹H NMR (400 MHz, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

### Step 3—3-(5-Bromo-3-methyl-2-oxo-benzimidazol-1-yl)piperidine-2,6-dione

To a mixture of 3-(5-bromo-3-methyl-2-oxo-benzimidazol-1-yl)-1-[(4-methoxyphenyl)methyl]piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### 3-[3-Methyl-2-oxo-5-(4-piperidyl)benzimidazol-1-yl]piperidine-2,6-dione (Intermediate AZL)

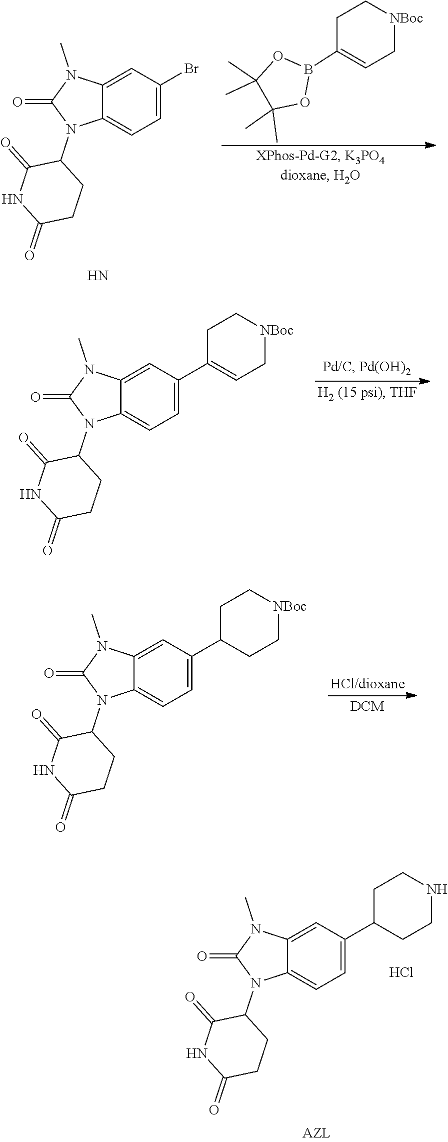

### Step 1—Tert-butyl 4-[1-(2,6-dioxo-3-piperidyl)-3-methyl-2-oxo-benzimidazol-4-yl]-3,6-dihydro-2H-pyridine-1-carboxylate

To a solution of 3-(5-bromo-3-methyl-2-oxo-benzimidazol-1-yl)piperidine-2,6-dione (5.00 g, 14.8 mmol, Intermediate HN), tert-butyl 4-(4,4,5,5-tetramethyl-1,3,2-dioxaborolan-2-yl)-3,6-dihydro-2H-pyridine-1-carboxylate (5.49 g, 17.7 mmol, CAS #286961-14-6), K₃PO₄(6.28 g, 29.6 mmol) and [2-(2-aminophenyl)phenyl]-chloro-palladium; dicyclohexyl-[3-(2,4,6-triisopropylphenyl)phenyl]phosphane (1.16 g, 1.48 mmol) in dioxane (100 mL) and H₂O (5.0 mL) was stirred at 80° C. for 4 hrs. On completion, the mixture filtered and the filtrate was concentrated in vacuo. The residue was purified by reversed phase flash (0.1% FA condition) to give the title compound (2.30 g, 53% yield) as white solid. ¹H NMR (400 MHz, DMSO-d₆) δ 11.09 (s, 1H), 7.27 (s, 1H), 7.14-7.04 (m, 2H), 6.11 (s, 1H), 5.36 (dd, J=12.8, 5.2 Hz, 1H), 4.01 (d, J=7.2 Hz, 2H), 3.55 (t, J=5.6 Hz, 2H), 3.35 (s, 3H), 2.95-2.83 (m, 1H), 2.73-2.59 (m, 2H), 2.06-1.95 (m, 2H), 1.46-1.39 (m, 9H), 1.17 (t, J=7.2 Hz, 1H). LC-MS (ESI⁺) m/z 441.2 (M+H)⁺.

### Step 2—Tert-butyl 4-[1-(2,6-dioxo-3-piperidyl)-3-methyl-2-oxo-benzimidazol-5-yl]piperidine-1-carboxylate

To a solution of tert-butyl 4-[1-(2,6-dioxo-3-piperidyl)-3-methyl-2-oxo-benzimidazol-5-yl]-3,6-dihydro-2H-pyridine-1-carboxylate (2.30 g, 5.22 mmol) in THF (150 mL) was added Pd/C (800 mg, 10 wt %) and Pd(OH)₂ (800 mg, 5.70 mmol) at 25° C. The reaction mixture was stirred at 60° C. for 16 hr under H₂ (15 psi). On completion, the reaction mixture was filtered with celite and the filtrate was concentrated in vacuo to give the title compound (2.30 g, 87% yield) as white solid. ¹H NMR (400 MHz, CDCl₃) δ 8.10 (s, 1H), 7.02-6.87 (m, 2H), 6.76 (d, J=8.0 Hz, 1H), 5.23 (dd, J=5.6, 12.6 Hz, 1H), 4.30-4.25 (m, 2H), 3.45 (s, 3H), 2.99-2.68 (m, 6H), 2.30-2.21 (m, 1H), 1.88-1.81 (m, 2H), 1.51 (s, 9H), 1.48-1.44 (m, 2H). LC-MS (ESI⁺) m/z 465.2 (M+23)+.

### Step 3—3-[3-Methyl-2-oxo-5-(4-piperidyl)benzimidazol-1-yl]piperidine-2,6-dione

To a mixture of tert-butyl 4-[1-(2,6-dioxo-3-piperidyl)-3-methyl-2-oxo-benzimidazol-5-yl]piperidine-1-carboxylate (300 mg, 678 umol) in DCM (3.0 mL) was added HCl/dioxane (4 M, 170 uL) in one portion at 25° C. under N₂. The mixture was stirred at 25° C. for 30 min. On completion, the reaction mixture was concentrated in vacuo to give the title compound (250 mg, 91% yield, HCl salt) as white solid. LC-MS (ESI⁺) m/z 343.1 (M+H)⁺.

### 6-Cyanopyrazolo[1,5-a]pyrimidine-3-carbonyl chloride (Intermediate BVQ)

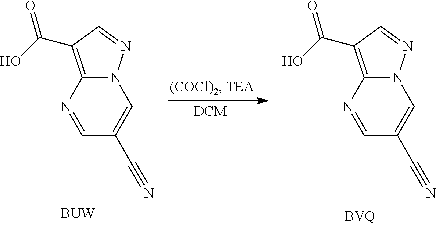 [SMILES: N#Cc1cnc2c(C(=O)O)cnn2c1 ; N#Cc1cnc2c(C(=O)O)cnn2c1 ; "BUW"]

To a solution of 6-cyanopyrazolo[1,5-a]pyrimidine-3-carboxylic acid (60.0 mg, 318 umol, Intermediate BUW) in DCM (2 mL) was added TEA (32.2 mg, 318 umol), then (COCl)₂ (40.4 mg, 318 umol) was added dropwise at 0° C. The mixture was then stirred at 20° C. for 0.5 hr. On completion, the mixture was concentrated in vacuo to give the title compound (65.0 mg, 98% yield) as brown solid.

### (1R,4r)-4-((Benzyloxy)methyl)cyclohexanecarbonyl chloride (Intermediate BAU)

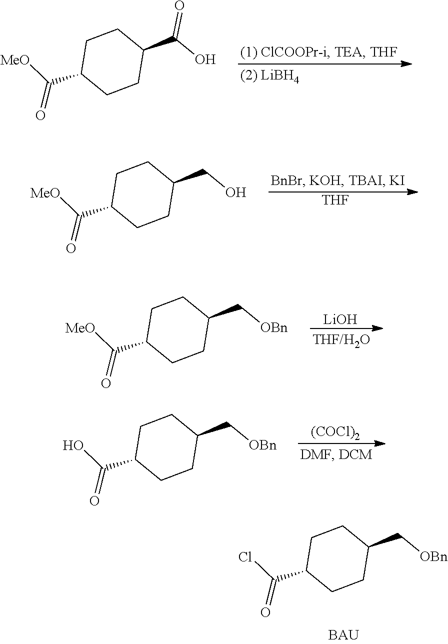

### Step 1—(1R,4r)-Methyl 4-(hydroxymethyl)cyclohexanecarboxylate

To a solution of 4-methoxycarbonylcyclohexanecarboxylic acid (20.0 g, 107 mmol, CAS #15177-67-0) in the THF (200 mL) was added Et₃N (21.7 g, 215 mmol, 29.9 mL) and isopropyl carbonochloridate (19.7 g, 161 mmol, 22.4 mL) at 0° C. The mixture was stirred at 25° C. for 1 hour. Then the mixture was filtered and the LiBH₄ (11.7 g, 537 mmol) was added in portion at 0° C. The mixture was stirred at 25° C. for 4 hours. On completion, the mixture was quenched by water (500 mL) and extracted with EA (3×1000 mL). The organic layers were dried over Na₂SO₄, filtered and concentrated in vacuo. The residue was purified by column chromatography to give the title compound (9.70 g, 52% yield) as colorless oil. ¹H NMR (400 MHz, CDCl₃) δ 3.67 (s, 3H), 3.47 (d, J=6.0 Hz, 2H), 2.26 (tt, J=3.6, 12.4 Hz, 1H), 2.06-1.99 (m, 2H), 1.88 (dd, J=3.2, 13.6 Hz, 2H), 1.56-1.39 (m, 3H), 1.07-0.93 (m, 2H).

### Step 2—(1R,4r)-Methyl 4-((benzyloxy)methyl)cyclohexanecarboxylate

To a solution of methyl 4-(hydroxymethyl)cyclohexanecarboxylate (9.70 g, 56.3 mmol) in the THF (100 mL) was added KOH (4.74 g, 84.5 mmol), TBAI (4.16 g, 11.3 mmol), KI (1.87 g, 11.3 mmol) and BnBr (14.5 g, 84.5 mmol, 10.0 mL). The mixture was stirred at 25° C. for 12 hours. On completion, the reaction mixture was filtered and concentrated in vacuo. The residue was purified by column chromatography to give the title compound (11.0 g, 74% yield) as colorless oil. ¹H NMR (400 MHz, CDCl₃) δ 7.39-7.27 (m, 5H), 4.50 (s, 2H), 3.67 (s, 3H), 3.29 (d, J=6.4 Hz, 2H), 2.25 (tt, J=3.6, 12.4 Hz, 1H), 2.04-1.98 (m, 2H), 1.91 (br dd, J=3.6, 13.6 Hz, 2H), 1.71-1.61 (m, 1H), 1.45-1.42 (m, 2H), 1.08-0.94 (m, 2H).

### Step 3—(1R,4r)-4-((benzyloxy)methyl)cyclohexanecarboxylic acid

To a solution of methyl 4-(benzyloxymethyl)cyclohexanecarboxylate (11.0 g, 41.9 mmol) in the THF (100 mL), MeOH (20 mL) and H₂O (20 mL) was added LiOH (5.02 g, 210 mmol). The mixture was stirred at 25° C. for 12 hours. On completion, the reaction mixture was concentrated in vacuo. The residue was diluted with water (100 mL) and washed with PE (200 mL). The water phase was acidified by HCl (aq, 1M) to pH=4. Then the mixture was extracted with DCM (3×200 mL). The organic layer was dried over Na₂SO₄, filtered and concentrated in vacuo to give the title compound (10.1 g, 97% yield) as colorless oil. ¹H NMR (400 MHz, CDCl₃) δ 7.41-7.26 (m, 5H), 4.50 (s, 2H), 3.30 (d, J=6.4 Hz, 2H), 2.28 (tt, J=3.6, 12.4 Hz, 1H), 2.05 (dd, J=2.8, 13.6 Hz, 2H), 1.92 (dd, J=2.8, 13.6 Hz, 2H), 1.65-1.62 (m, 1H), 1.46 (dq, J=3.6, 12.8 Hz, 2H), 1.11-0.95 (m, 2H).

### Step 4—(1R,4r)-4-((Benzyloxy)methyl)cyclohexanecarbonyl chloride

To a solution of 4-(benzyloxymethyl)cyclohexanecarboxylic acid (10.0 g, 40.3 mmol) in the DCM (100 mL) was added DMF (294 mg, 4.03 mmol) and (COCl)₂ (7.67 g, 60.4 mmol, 5.29 mL) in portion at 0° C. The mixture was stirred at 0° C. for 2 hrs. On completion, the reaction mixture was concentrated in vacuo to give the title compound (10.7 g, 99% yield) as yellow oil.

### N-[2-(4-formylcyclohexyl)-1,3-benzothiazol-6-yl]-6-(trifluoromethyl)pyridine-2-carboxamide (Intermediate BTI)

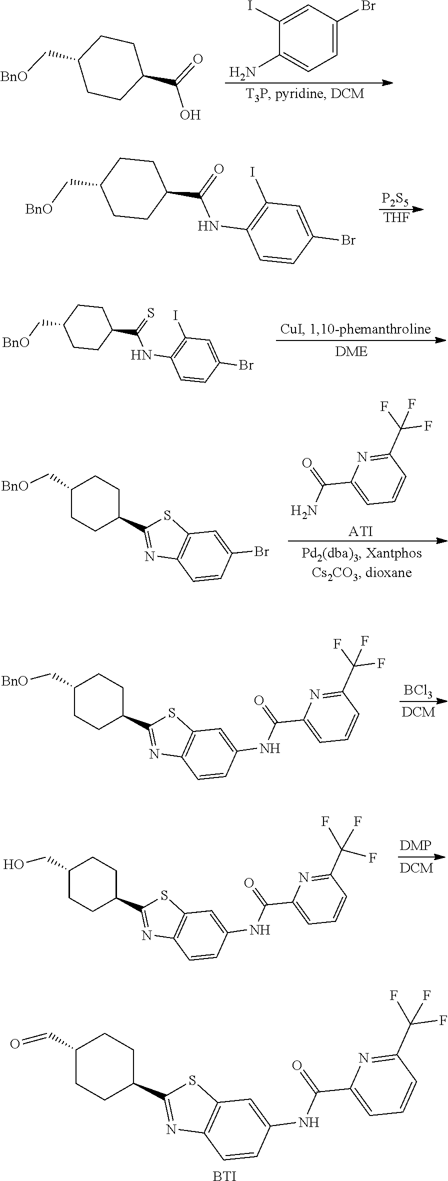

### Step 1—4-(benzyloxymethyl)-N-(4-bromo-2-iodo-phenyl)cyclohexanecarboxamide

To a mixture of 4-(benzyloxymethyl)cyclohexanecarboxylic acid (10.0 g, 40.2 mmol, synthesized via Steps 1-3 of Intermediate BAU) and 4-bromo-2-iodo-aniline (6.00 g, 20.1 mmol, CAS #66416-72-6) in DCM (100 mL) was added pyridine (3.19 g, 40.2 mmol) and T₃P (76.8 g, 120 mmol). The reaction mixture was stirred at 50° C. for 24 hr. On completion, the reaction mixture was filtered and concentrated in vacuo. The residue was purified by reverse phase (0.1% FA condition) to give the title compound (4.27 g, 20% yield) as white solid. ¹H NMR (400 MHz, DMSO-d₆) δ 9.36-9.29 (m, 1H), 8.04 (d, J=2.0 Hz, 1H), 7.55 (dd, J=2.0, 8.4 Hz, 1H), 7.46-7.24 (m, 6H), 4.45 (s, 2H), 3.39-3.28 (m, 2H), 3.27 (d, J=6.4 Hz, 2H), 2.42-2.29 (m, 1H), 1.96-1.89 (m, 2H), 1.87-1.78 (m, 2H), 1.47-1.42 (m, 1H), 1.08-0.95 (m, 2H).

### Step 2—4-(Benzyloxymethyl)-N-(4-bromo-2-iodo-phenyl)cyclohexanecarbothioamide

To a mixture of 4-(benzyloxymethyl)-N-(4-bromo-2-iodo-phenyl)cyclohexanecarboxamide (4.27 g, 8.08 mmol) in THF (30 mL) was added P₂S₅(2.70 g, 12.1 mmol). The reaction mixture was stirred at 25° C. for 12 hours. On completion, the reaction mixture was quenched with water (0.05 mL), then the reaction mixture was filtered and concentrated in vacuo. The residue was diluted with water (30 mL) and extracted with EA (2×200 mL). The combined organic layer was dried over Na₂SO₄, filtered and concentrated in vacuo to give the title compound (946 mg, 21% yield) as white solid. LC-MS (ESI⁺) m/z 545.3 (M+H)⁺.

### Step 3—2-[4-(Benzyloxymethyl)cyclohexyl]-6-bromo-1,3-benzothiazole

To a mixture of 4-(benzyloxymethyl)-N-(4-bromo-2-iodo-phenyl)cyclohexanecarbothioamide (845 mg, 1.55 mmol) in DME (2 mL) was added CuI (29.5 mg, 155 umol) and 1,10-phenanthroline (27.9 mg, 155 umol) in N₂. The reaction mixture was stirred at 40° C. for 12 hours. On completion, the reaction mixture was concentrated in vacuo. The residue was purified by column chromatography to give the title compound (429 mg, 66% yield) as yellow solid. ¹H NMR (400 MHz, DMSO-d₆) δ 8.34 (d, J=2.0 Hz, 1H), 7.86 (d, J=8.8 Hz, 1H), 7.62 (dd, J=2.0, 8.4 Hz, 1H), 7.38-7.25 (m, 5H), 4.46 (s, 2H), 3.29 (d, J=6.4 Hz, 2H), 3.10-3.00 (m, 1H), 2.22-2.10 (m, 2H), 1.93-1.84 (m, 2H), 1.71-1.50 (m, 3H), 1.25-1.08 (m, 2H).

### Step 4—N-[2-[4-(benzyloxymethyl)cyclohexyl]-1,3-benzothiazol-6-yl]-6-(trifluoromethyl)pyridine-2-carboxamide

To a mixture 2-[4-(benzyloxymethyl)cyclohexyl]-6-bromo-1,3-benzothiazole (389 mg, 934.26 umol) and 6-(trifluoromethyl)pyridine-2-carboxamide (195 mg, 1.03 mmol, Intermediate ATI) in dioxane (5 mL) was added Cs₂CO₃ (913 mg, 2.80 mmol), Pd₂(dba)₃ (85.5 mg, 93.4 umol) and Xantphos (54.0 mg, 93.4 umol). Then the reaction mixture was stirred at 80° C. for 2 hours. On completion, the residue was diluted with water (30 mL) and extracted with EA (2×50 mL). The combined organic layer was dried over Na₂SO₄, filtered and concentrated in vacuo. The residue was purified by reverse phase (0.1% FA condition) to give the title compound (188 mg, 38% yield) as yellow solid. LC-MS (ESI⁺) m/z 526.1 (M+H)⁺.

### Step 5—N-[2-[4-(hydroxymethyl)cyclohexyl]-1,3-benzothiazol-6-yl]-6-(trifluoromethyl)pyridine-2-carboxamide

To a mixture of N-[2-[4-(benzyloxymethyl)cyclohexyl]-1,3-benzothiazol-6-yl]-6-(trifluoromethyl) pyridine-2-carboxamide (178 mg, 338 umol) in DCM (3 mL) was added BCl₃ (1 M, 677 uL). Then the reaction mixture was stirred at 0° C. for 1 hour. On completion, the reaction mixture was quenched with water (0.05 mL) and concentrated in vacuo to give the title compound (112 mg, 75% yield) as yellow solid. ¹H NMR (400 MHz, DMSO-d₆) δ 10.62 (s, 1H), 8.60 (d, J=1.6 Hz, 1H), 8.43-8.34 (m, 2H), 8.19 (dd, J=1.2, 7.6 Hz, 1H), 7.95-7.87 (m, 2H), 3.29-3.24 (m, 3H), 3.09-3.00 (m, 1H), 2.22-2.15 (m, 2H), 1.92-1.84 (m, 2H), 1.64-1.52 (m, 2H), 1.50-1.39 (m, 1H), 1.16-1.04 (m, 2H).

### Step 6—2-N-[2-(4-formylcyclohexyl)-1,3-benzothiazol-6-yl]-6-(trifluoromethyl)pyridine-2-carboxamide

To a mixture of N-[2-[4-(hydroxymethyl)cyclohexyl]-1,3-benzothiazol-6-yl]-6-(trifluoromethyl) pyridine-2-carboxamide (102 mg, 234 umol) in DCM (2 mL) was added DMP (129 mg, 304 umol). Then the reaction mixture was stirred at 25° C. for 1 hours. On completion, the reaction mixture was quenched with saturated Na₂S₂O₃ (8 mL) and saturated NaHCO₃ (8 mL) at 25° C., and then the mixture was stirred for 30 minutes. The mixture was then extracted with DCM (2×20 mL). The combined organic layer was dried over Na₂SO₄, filtered and concentrated in vacuo to give the title compound (100 mg, 98% yield) as black brown solid. ¹H NMR (400 MHz, DMSO-d₆) δ 10.62 (s, 1H), 9.68-9.59 (m, 1H), 8.63-8.57 (m, 1H), 8.46-8.34 (m, 2H), 8.19 (d, J=7.6 Hz, 1H), 7.98-7.86 (m, 2H), 3.15-3.04 (m, 1H), 2.45-2.36 (m, 1H), 2.27-2.22 (m, 1H), 2.11-1.97 (m, 3H), 1.74-1.61 (m, 2H), 1.47-1.34 (m, 2H). LC-MS (ESI⁺) m/z 434.0 (M+H)⁺.

### 2-(2,6-dioxo-3-piperidyl)-5-fluoro-isoindoline-1,3-dione (Intermediate HX)

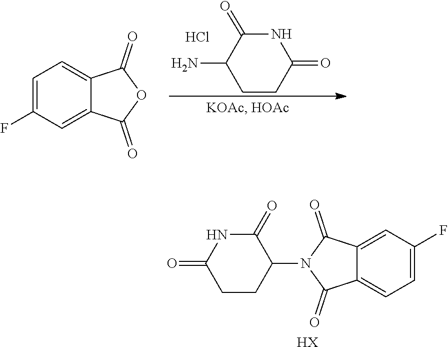

To a mixture of 3-aminopiperidine-2,6-dione (10.8 g, 65.8 mmol, HCl) and KOAc (18.2 g, 185 mmol) in HOAc (160 mL) was added 5-fluoroisobenzofuran-1,3-dione (9.95 g, 59.9 mmol, CAS #319-03-9). Then the mixture was stirred at 90° C. for 16 hours. On completion, the reaction mixture was cooled to 25° C. and diluted with water (600 mL), and then stirred at 0° C. for 0.5 hour then filtered. The filter cake was dried in vacuo to give the title compound (14.0 g, 84% yield) as black brown solid. ¹H NMR (400 MHz, DMSO-d₆) δ 11.15 (s, 1H), 8.01 (dd, J=4.4, 8.0 Hz, 1H), 7.84 (dd, J=2.4, 7.6 Hz, 1H), 7.76-7.67 (m, 1H), 5.17 (dd, J=5.6, 12.8 Hz, 1H), 2.97-2.83 (m, 1H), 2.65-2.51 (m, 2H), 2.13-2.03 (m, 1H).

### 5-[2-(2-Azaspiro[3.3]heptan-6-yl)ethylamino]-2-(2,6-dioxo-3-piperidyl)isoindoline-1,3-dione (Intermediate BTH)

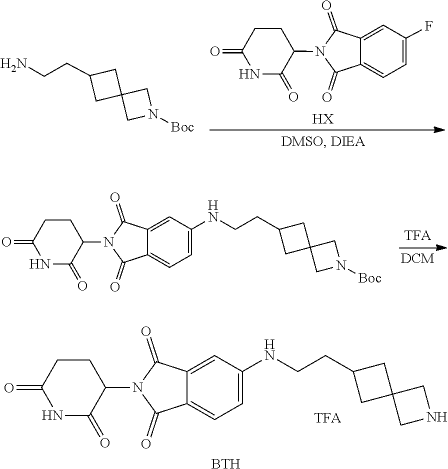

### Step 1—Tert-butyl 6-[2-[[2-(2,6-dioxo-3-piperidyl)-1,3-dioxo-isoindolin-5-yl]amino]ethyl]-2-azaspiro [3.3]heptane-2-carboxylate

To a solution of tert-butyl 6-(2-aminoethyl)-2-azaspiro[3.3]heptane-2-carboxylate (140 mg, 582 umol, Intermediate ATG) in DMSO (3 mL) was added 2-(2,6-dioxo-3-piperidyl)-5-fluoro-isoindoline-1,3-dione (193 mg, 699 umol, Intermediate HX) and DIEA (225 mg, 1.75 mmol). The mixture was degassed and purged with N₂ 3 times, and then the mixture was stirred at 130° C. for 3 hrs under N₂ atmosphere. On completion, the mixture was filtered and the filtrate was concentrated under reduced pressure to afford a residue. The residue was purified by reversed-phase HPLC (0.1% FA condition) to give the title compound (40.0 mg, 14% yield) as a green solid. ¹H NMR (400 MHz, CHLOROFORM-d) δ 7.97 (s, 1H), 7.63 (d, J=8.4 Hz, 1H), 6.97 (s, 1H), 6.75 (d, J=8.4 Hz, 1H), 4.94 (dd, J=5.6, 12.4 Hz, 1H), 3.95 (s, 2H), 3.82 (s, 2H), 3.16 (t, J=7.2 Hz, 2H), 2.93-2.87 (m, 1H), 2.85-2.72 (m, 2H), 2.37-2.31 (m, 2H), 2.28-2.19 (m, 1H), 2.16-2.10 (m, 1H), 1.87-1.82 (m, 2H), 1.77-1.70 (m, 2H), 1.44 (s, 9H).

### Step 2—5-[2-(2-Azaspiro[3.3]heptan-6-yl)ethylamino]-2-(2,6-dioxo-3-piperidyl)isoindoline-1,3-dione

To a solution of tert-butyl 6-[2-[[2-(2,6-dioxo-3-piperidyl)-1,3-dioxo-isoindolin-5-yl]amino]ethyl]-2-azaspiro[3.3]heptane-2-carboxylate (40.0 mg, 80.5 umol) in DCM (2 mL) was added TFA (770 mg, 6.75 mmol) at 25° C. The mixture was stirred at 25° C. for 1 hr. On completion, the reaction mixture was concentrated under reduced pressure to give the title compound (40.0 mg, 97% yield, TFA salt) as yellow oil. LC-MS (ESI⁺) m/z 397.2 (M+H)⁺.

### (1R,4r)-4-((Benzyloxy)methyl)cyclohexanecarbonyl chloride (Intermediate BAU)

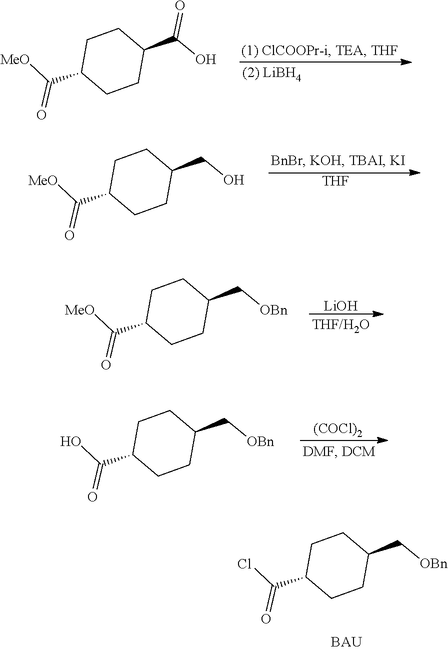

### Step 1—(1R,4r)-Methyl 4-(hydroxymethyl)cyclohexanecarboxylate

To a solution of 4-methoxycarbonylcyclohexanecarboxylic acid (20.0 g, 107 mmol, CAS #15177-67-0) in the THF (200 mL) was added Et₃N (21.7 g, 215 mmol, 29.9 mL) and isopropyl carbonochloridate (19.7 g, 161 mmol, 22.4 mL) at 0° C. The mixture was stirred at 25° C. for 1 hour. Then the mixture was filtered and the LiBH₄ (11.7 g, 537 mmol) was added in portion at 0° C. The mixture was stirred at 25° C. for 4 hours. On completion, the mixture was quenched by water (500 mL) and extracted with EA (3×1000 mL). The organic layers were dried over Na₂SO₄, filtered and concentrated in vacuo. The residue was purified by column chromatography to give the title compound (9.70 g, 52% yield) as colorless oil. ¹H NMR (400 MHz, CDCl₃) δ 3.67 (s, 3H), 3.47 (d, J=6.0 Hz, 2H), 2.26 (tt, J=3.6, 12.4 Hz, 1H), 2.06-1.99 (m, 2H), 1.88 (dd, J=3.2, 13.6 Hz, 2H), 1.56-1.39 (m, 3H), 1.07-0.93 (m, 2H).

### Step 2—(1R,4r)-Methyl 4-((benzyloxy)methyl)cyclohexanecarboxylate

To a solution of methyl 4-(hydroxymethyl)cyclohexanecarboxylate (9.70 g, 56.3 mmol) in the THF (100 mL) was added KOH (4.74 g, 84.5 mmol), TBAI (4.16 g, 11.3 mmol), KI (1.87 g, 11.3 mmol) and BnBr (14.5 g, 84.5 mmol, 10.0 mL). The mixture was stirred at 25° C. for 12 hours. On completion, the reaction mixture was filtered and concentrated in vacuo. The residue was purified by column chromatography to give the title compound (11.0 g, 74% yield) as colorless oil. ¹H NMR (400 MHz, CDCl₃) δ 7.39-7.27 (m, 5H), 4.50 (s, 2H), 3.67 (s, 3H), 3.29 (d, J=6.4 Hz, 2H), 2.25 (tt, J=3.6, 12.4 Hz, 1H), 2.04-1.98 (m, 2H), 1.91 (br dd, J=3.6, 13.6 Hz, 2H), 1.71-1.61 (m, 1H), 1.45-1.42 (m, 2H), 1.08-0.94 (m, 2H).

### Step 3—(1R,4r)-4-((benzyloxy)methyl)cyclohexanecarboxylic acid

To a solution of methyl 4-(benzyloxymethyl)cyclohexanecarboxylate (11.0 g, 41.9 mmol) in the THF (100 mL), MeOH (20 mL) and H₂O (20 mL) was added LiOH (5.02 g, 210 mmol). The mixture was stirred at 25° C. for 12 hours. On completion, the reaction mixture was concentrated in vacuo. The residue was diluted with water (100 mL) and washed with PE (200 mL). The water phase was acidified by HCl (aq, 1M) to pH=4. Then the mixture was extracted with DCM (3×200 mL). The organic layer was dried over Na₂SO₄, filtered and concentrated in vacuo to give the title compound (10.1 g, 97% yield) as colorless oil. ¹H NMR (400 MHz, CDCl₃) δ7.41-7.26 (m, 5H), 4.50 (s, 2H), 3.30 (d, J=6.4 Hz, 2H), 2.28 (tt, J=3.6, 12.4 Hz, 1H), 2.05 (dd, J=2.8, 13.6 Hz, 2H), 1.92 (dd, J=2.8, 13.6 Hz, 2H), 1.65-1.62 (m, 1H), 1.46 (dq, J=3.6, 12.8 Hz, 2H), 1.11-0.95 (m, 2H).

### Step 4—(1R,4r)-4-((Benzyloxy)methyl)cyclohexanecarbonyl chloride

To a solution of 4-(benzyloxymethyl)cyclohexanecarboxylic acid (10.0 g, 40.3 mmol) in the DCM (100 mL) was added DMF (294 mg, 4.03 mmol) and (COCl)₂ (7.67 g, 60.4 mmol, 5.29 mL) in portion at 0° C. The mixture was stirred at 0° C. for 2 hrs. On completion, the reaction mixture was concentrated in vacuo to give the title compound (10.7 g, 99% yield) as yellow oil.

### Methyl 5-amino-2-bromo-4-iodo-benzoate (Intermediate BAV)

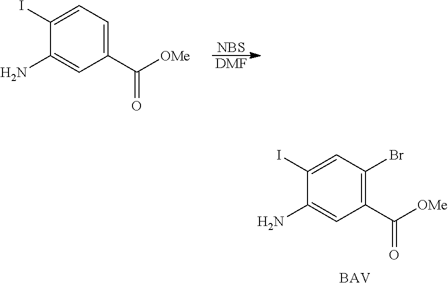

To a solution of methyl 3-amino-4-iodo-benzoate (5.00 g, 18.1 mmol, CAS #412947-54-7) in DMF (25 mL) was added NBS (3.28 g, 18.4 mmol). The mixture was stirred at 0° C. for 2 hours. On completion, the mixture was poured into 500 mL water and a solid was obtained. The mixture was filtered then the filtered cake was washed with water (3×50 mL) and dried in vacuo to give the title compound (6.00 g, 93% yield) as yellow solid. ¹H NMR (400 MHz, DMSO-d₆) δ 7.84 (s, 1H), 7.13 (s, 1H), 5.66 (br s, 2H), 3.81 (s, 3H).

### Methyl 6-bromo-2-[4-(hydroxymethyl)cyclohexyl]-1,3-benzothiazole-5-carboxylate (Intermediate BAW)

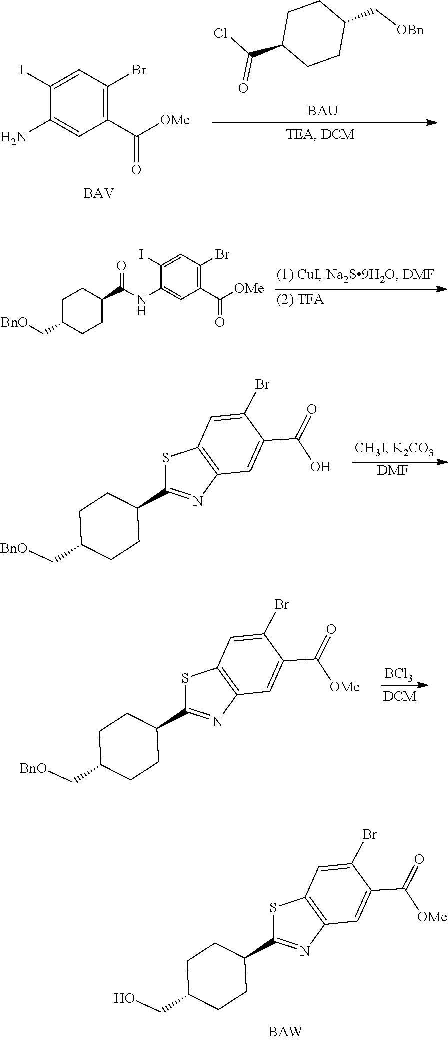

### Step 1—Methyl 5-[[4-(benzyloxymethyl)cyclohexanecarbonyl]amino]-2-bromo-4-iodo-benzoate

To a solution of methyl 5-amino-2-bromo-4-iodo-benzoate (707 mg, 1.9 mmol, Intermediate BAV) in DCM (10 mL) was added Et₃N (603 mg, 5.96 mmol). Then a mixture of 4-(benzyloxymethyl)cyclohexane carbonyl chloride (530 mg, 1.99 mmol, Intermediate BAU) in DCM (20 mL) was added to the reaction mixture. The mixture was stirred at 0° C. for 2 hours. On completion, the mixture was concentrated in vacuo. The residue was diluted with water (50 mL) and extracted with EA (3×100 mL). The combined organic layers were washed with brine (50 mL), dried over Na₂SO₄, filtered and concentrated of most solvent. Then the solid was precipitated out, then filtered, the cake was dried in vacuo to give the title compound (660 mg, 56% yield) as white solid. ¹H NMR (400 MHz, CDCl₃) δ 8.76 (d, J=1.6 Hz, 1H), 8.09 (d, J=1.6 Hz, 1H), 7.52 (s, 1H), 7.41-7.27 (m, 5H), 4.52 (d, J=1.6 Hz, 2H), 3.92 (d, J=1.6 Hz, 3H), 3.34 (dd, J=1.6, 6.0 Hz, 2H), 2.35-2.24 (m, 1H), 2.12 (d, J=13.2 Hz, 2H), 2.00 (d, J=13.2 Hz, 2H), 1.77-1.58 (m, 3H), 1.19-1.05 (m, 2H).

### Step 2—2-[4-(Benzyloxymethyl)cyclohexyl]-6-bromo-1,3-benzothiazole-5-carboxylic acid

To a solution of methyl 5-[[4-(benzyloxymethyl)cyclohexanecarbonyl]amino]-2-bromo-4-iodo-benzoate (5.60 g, 9.55 mmol) in DMF (50 mL) was added CuI (363 mg, 1.91 mmol) and Na₂S·9H₂O (13.7 g, 57.3 mmol). The mixture was stirred at 80° C. for 6 hours, and then cooled to rt. Then TFA (15.4 g, 135 mmol) was added to the mixture and the mixture was stirred at 25° C. for 6 hours. On completion, the residue was diluted with water (100 mL) and extracted with EA (3×100 mL). The combined organic layers were washed with brine (100 mL), dried over Na₂SO₄, filtered and concentrated in vacuo to give the title compound (4.00 g, 56% yield) as yellow oil. LC-MS (ESI+) m/z 462.1 (M+3)⁺.

### Step 3—Methyl 2-[4-(benzyloxymethyl)cyclohexyl]-6-bromo-1,3-benzothiazole-5-carboxylate

To a solution of 2-[4-(benzyloxymethyl)cyclohexyl]-6-bromo-1,3-benzothiazole-5-carboxylic acid (4.00 g, 8.69 mmol) in DMF (20 mL) was added CH₃I (2.47 g, 17.3 mmol) and K₂CO₃ (2.40 g, 17.3 mmol). The mixture was stirred at 15° C. for 2 hours. On completion, the mixture was filtered and concentrated in vacuo. The residue was purified by flash silica gel chromatography (PE:EA 3:1) to give title compound (3.00 g, 72% yield) as white solid. ¹H NMR (400 MHz, CDCl₃) δ 8.31 (s, 1H), 8.05 (s, 1H), 7.31-7.21 (m, 5H), 4.44 (s, 2H), 3.88 (s, 3H), 3.27 (d, J=6.0 Hz, 2H), 2.97 (t, J=12.0 Hz, 1H), 2.87 (s, 5H), 2.80 (s, 5H), 2.19 (d, J=12.4 Hz, 2H), 1.95 (d, J=13.6 Hz, 2H), 1.73-1.65 (m, 1H), 1.58 (q, J=12.8 Hz, 2H), 1.20-1.07 (m, 2H).

### Step 4—Methyl 6-bromo-2-[4-(hydroxymethyl)cyclohexyl]-1,3-benzothiazole-5-carboxylate

To a solution of methyl 2-[4-(benzyloxymethyl)cyclohexyl]-6-bromo-1,3-benzothiazole-5-carboxylate (2.00 g, 4.22 mmol) in DCM (40 mL) was added BCl₃ (9.88 g, 84.3 mmol). The mixture was stirred at 25° C. for 2 hours. On completion, to the mixture was added sat.NaHCO₃. aq (50 mL) then extracted with DCM (3×50 mL). The combined organic layers were washed with brine (100 mL), dried over Na₂SO₄, filtered and concentrated in vacuo to give the title compound (1.60 g, 90% yield) as white solid. ¹H NMR (400 MHz, CDCl₃) δ 8.48 (s, 1H), 8.21-8.13 (m, 1H), 3.98 (s, 3H), 3.55 (d, J=6.0 Hz, 2H), 3.25-3.12 (m, 1H), 2.42-2.26 (m, 2H), 2.09-1.98 (m, 2H), 1.78-1.62 (m, 3H), 1.29-1.16 (m, 2H).

### N-[2-(4-formylcyclohexyl)-5-(1-hydroxy-1-methyl-ethyl)-1,3-benzothiazol-6-yl]-6-(trifluoromethyl)pyridine-2-carboxamide (Intermediate BAX)

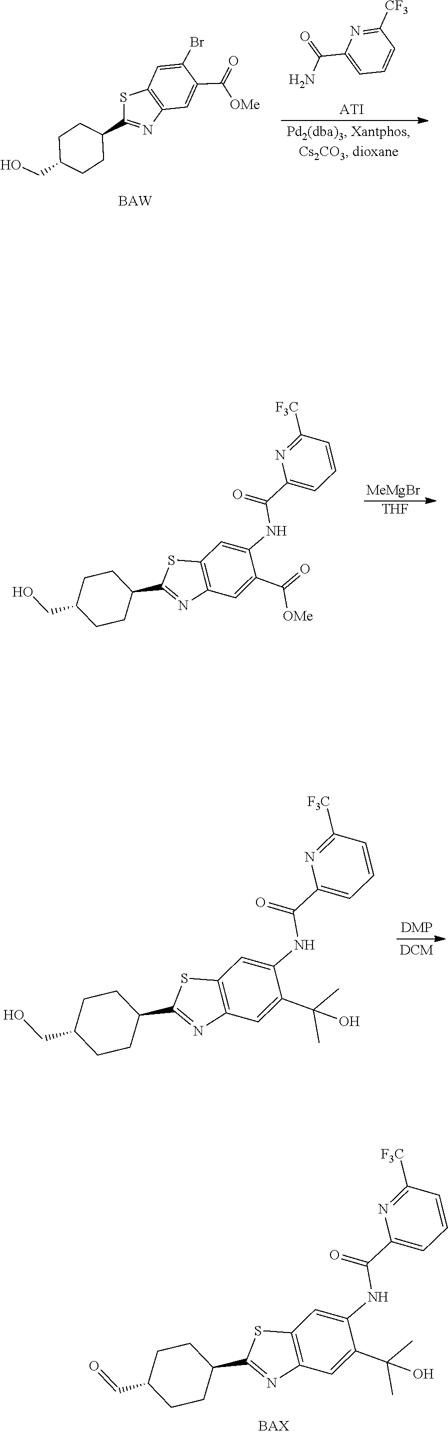

### Step 1—Methyl 2-[4-(hydroxymethyl)cyclohexyl]-6-[[6-(trifluoromethyl)pyridine-2-carbonyl]amino]-1,3-benzothiazole-5-carboxylate

To a solution of methyl 6-bromo-2-[4-(hydroxymethyl)cyclohexyl]-1,3-benzothiazole-5-carboxylate (300 mg, 780 umol, Intermediate BAW) and 6-(trifluoromethyl)pyridine-2-carboxamide (163 mg, 858 umol, Intermediate ATI) in dioxane (30 mL) was added Xantphos (90.3 mg, 156 umol), Cs₂CO₃ (763 mg, 2.34 mmol) and Pd₂(dba)₃ (71.4 mg, 78.1 umol) at 25° C. The mixture was stirred at 80° C. for 12 hrs under N₂. On completion, the mixture was filtered with celite and concentrated in vacuo. The residue was purified by column chromatography to give title compound (120 mg, 31% yield) as yellow solid. ¹H NMR (400 MHz, DMSO-d₆) δ 12.82 (s, 1H), 9.44 (s, 1H), 8.54 (s, 1H), 8.50-8.46 (m, 1H), 8.45-8.38 (m, 1H), 8.23 (d, J=7.8 Hz, 1H), 4.53-4.40 (m, 1H), 3.98 (s, 3H), 3.27 (t, J=5.6 Hz, 2H), 3.08 (s, 1H), 2.19 (d, J=13.0 Hz, 2H), 1.93-1.83 (m, 2H), 1.66-1.51 (m, 2H), 1.48-1.38 (m, 1H), 1.18-1.05 (m, 2H).

### Step 2—N-[2-[4-(hydroxymethyl)cyclohexyl]-5-(1-hydroxy-1-methyl-ethyl)-1,3-benzothiazol-6-yl]-6-(trifluoromethyl)pyridine-2-carboxamide

To a solution of methyl 2-[4-(hydroxymethyl)cyclohexyl]-6-[[6-(trifluoromethyl)pyridine-2-carbonyl]amino]-1,3-benzothiazole-5-carboxylate (120 mg, 243 umol) in THF (10 mL) was added MeMgBr (3 M, 405 uL). The mixture was stirred at 0° C. for 2 hours. The reaction mixture was quenched by addition sat. NH₄Cl (10 mL) at 0° C., and then diluted with water (50 mL) and extracted with EA (3×50 mL). The combined organic layers were washed with brine (100 mL), dried over Na₂SO₄, filtered and concentrated in vacuo to give a residue. The residue was purified by prep-HPLC (column: Phenomenex Synergi C18 150*25*10 um; mobile phase: [water (0.225% FA)-ACN]; B %: 44%-74%, 10 min) to give the title compound (80.0 mg, 60% yield) as white solid. ¹H NMR (400 MHz, DMSO-d₆) δ 12.56 (s, 1H), 9.07 (s, 1H), 8.51-8.45 (m, 1H), 8.39 (t, J=8.0 Hz, 1H), 8.20 (d, J=7.6 Hz, 1H), 7.94-7.88 (m, 1H), 6.08 (s, 1H), 4.46 (t, J=5.2 Hz, 1H), 3.28 (t, J=5.6 Hz, 2H), 3.10-3.00 (m, 1H), 2.19 (d, J=11.2 Hz, 2H), 1.94-1.84 (m, 2H), 1.64 (s, 6H), 1.61-1.53 (m, 2H), 1.50-1.40 (m, 1H), 1.19-1.06 (m, 2H).

### Step 3—N-[2-(4-formylcyclohexyl)-5-(1-hydroxy-1-methyl-ethyl)-1,3-benzothiazol-6-yl]-6-(trifluoromethyl)pyridine-2-carboxamide

To a solution of N-[2-[4-(hydroxymethyl)cyclohexyl]-5-(1-hydroxy-1-methyl-ethyl)-1,3-benzothiazol-6-yl]-6-(trifluoromethyl)pyridine-2-carboxamide (50.0 mg, 101 umol) in DCM (10 mL) was added DMP (51.5 mg, 121 umol). The mixture was stirred at 25° C. for 2 hours. On completion, the mixture was added 10 mL sat. NaHCO₃ and 10 mL sat. Na₂S₂O₃, then extracted with DCM (3×50 mL). The combined organic layers were washed with brine (100 mL), dried over Na₂SO₄, filtered and concentrated in vacuo to give the title compound (60.0 mg, 90% yield) as yellow solid. LC-MS (ESI⁺) m/z 492.2 (M+1)⁺.

### 2-(2,6-Dioxo-3-piperidyl)-4-[[3-(methylamino)cyclobutyl]amino]isoindoline-1,3-dione (Intermediate AOW)

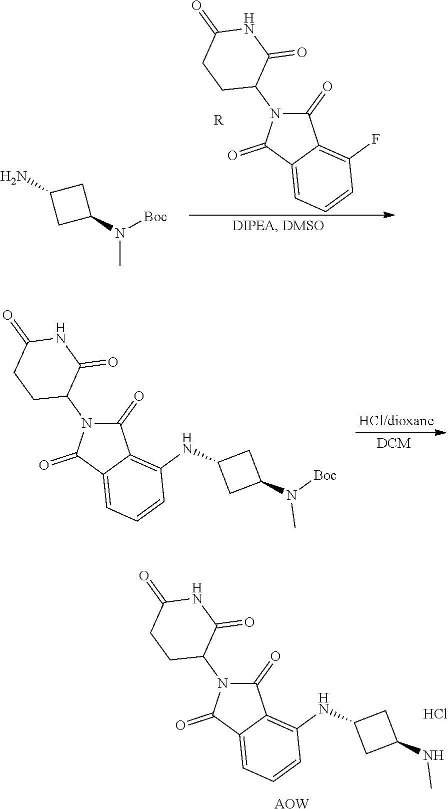

### Step 1—Tert-butyl N-[3-[[2-(2,6-dioxo-3-piperidyl)-1,3-dioxo-isoindolin-4-yl]amino]cyclobutyl]-N-methyl-carbamate

To a solution of tert-butyl N-(3-aminocyclobutyl)-N-methyl-carbamate (250 mg, 1.25 mmol, CAS #1392803-14-3) and 2-(2,6-dioxo-3-piperidyl)-4-fluoro-isoindoline-1,3-dione (362 mg, 1.31 mmol, Intermediate R) in DMSO (12 mL) was added DIPEA (322 mg, 2.50 mmol) at 25° C. The reaction mixture was stirred at 130° C. for 3 hrs. On completion, the reaction mixture was concentrated in vacuo. The residue was purified by reverse phase (0.1% FA condition) to give the title compound (500 mg, 87% yield) as a yellow solid. ¹H NMR (400 MHz, DMSO-d₆) δ 11.09 (s, 1H), 7.60 (dd, J=7.2, 8.4 Hz, 1H), 7.09 (d, J=6.8 Hz, 1H), 6.89 (d, J=8.4 Hz, 1H), 6.55 (d, J=5.6 Hz, 1H), 5.07 (dd, J=5.2, 12.8 Hz, 1H), 4.77-4.52 (m, 1H), 4.15-3.95 (m, 1H), 2.96-2.84 (m, 1H), 2.82 (s, 3H), 2.66-2.52 (m, 4H), 2.20 (t, J=8.8 Hz, 2H), 2.10-1.97 (m, 1H), 1.39 (s, 9H); LC-MS (ESI+) m/z 457.0 (M+H)⁺.

### Step 2—2-(2,6-Dioxo-3-piperidyl)-4-[[3-(methylamino)cyclobutyl]amino]isoindoline-1,3-dione

To a solution of tert-butyl N-[3-[[2-(2,6-dioxo-3-piperidyl)-1,3-dioxo-isoindolin-4-yl]amino]cyclobutyl]-N-methyl-carbamate (80.0 mg, 175 umol) in DCM (5 mL) was added HCl/dioxane (4 M, 2 mL) at 25° C. The mixture was stirred at 25° C. for 2 hrs. The mixture was stirred at 25° C. for 2 hrs. On completion, the reaction mixture was concentrated in vacuo to give the title compound (68 mg, 98% yield, HCl salt) as a yellow solid. LC-MS (ESI⁺) m/z 357.2 (M+H)⁺.

### Benzyl N-[3-(3-aminocyclobutoxy)propyl]-N-methyl-carbamate (Intermediate AOY)

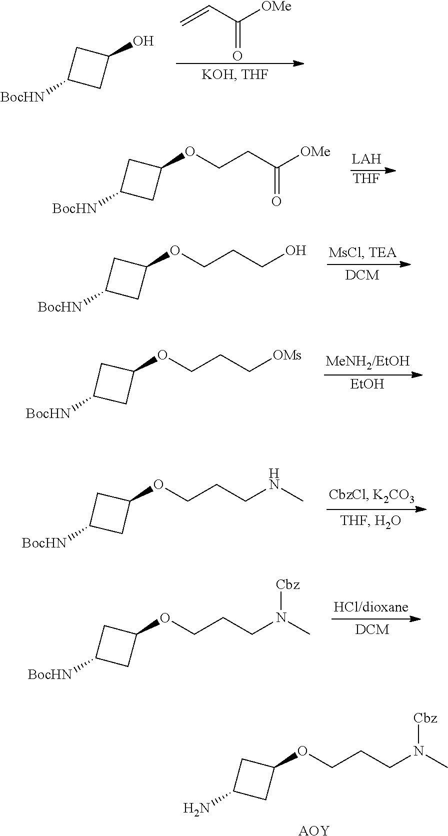

### Step 1—Methyl 3-[3-(tert-butoxycarbonylamino)cyclobutoxy]propanoate

To a solution of tert-butyl N-(3-hydroxycyclobutyl)carbamate (2.50 g, 13.3 mmol, CAS #389890-42-0) and methyl prop-2-enoate (2.30 g, 26.7 mmol, CAS #96-33-3) in THF (25 mL) was added KOH (74.9 mg, 1.34 mmol). The reaction mixture was stirred at 20° C. for 16 hrs. On completion, the reaction mixture was diluted with water (80 mL) and extracted with EA (3×80 mL). The combined organic layers were washed with brine (2×50 mL), dried over Na₂SO₄, filtered and concentrated in vacuo. The residue was purified by silica gel chromatography (PE:EA=8:1) to give the title compound (1.80 g, 49% yield) as colorless oil. ¹H NMR (400 MHz, CDCl₃) δ 4.81-4.59 (m, 1H), 4.26-4.03 (m, 2H), 3.70 (s, 3H), 3.59 (t, J=6.4 Hz, 2H), 2.57 (t, J=6.4 Hz, 2H), 2.42-2.30 (m, 2H), 2.19-2.06 (m, 2H), 1.44 (s, 9H).

### Step 2—Tert-butyl N-[3-(3-hydroxypropoxy)cyclobutyl]carbamate

To a solution of methyl 3-[3-(tert-butoxycarbonylamino)cyclobutoxy]propanoate (1.80 g, 6.59 mmol) in THF (20 mL) was added LAH (274 mg, 7.24 mmol). The reaction mixture was stirred at 0° C. for 1 hr. On completion, the reaction mixture was quenched by water (0.25 mL), added 15% NaOH (0.3 mL), water (0.8 mL), diluted with EA (50 mL), dried over Na₂SO₄, filtered and concentrated in vacuo to give the title compound (1.50 g, 92.% yield) as yellow oil. ¹H NMR (400 MHz, CDCl₃) δ 4.70 (s, 1H), 4.54-4.41 (m, 1H), 4.27-4.14 (m, 1H), 4.12-4.04 (m, 1H), 3.81-3.74 (m, 2H), 3.50 (t, J=6.0 Hz, 2H), 2.37-2.30 (m, 2H), 2.24-2.14 (m, 2H), 1.86-1.80 (m, 2H), 1.44 (s, 9H).

### Step 3—3-[3-(Tert-butoxycarbonylamino)cyclobutoxy]propyl methanesulfonate

To a solution of tert-butyl N-[3-(3-hydroxypropoxy)cyclobutyl]carbamate (1.50 g, 6.11 mmol) and TEA (928 mg, 9.17 mmol) in DCM (20 mL) was added MsCl (840 mg, 7.34 mmol) at 0° C. The reaction mixture was stirred at 0° C. for 1 hr. On completion, the reaction mixture was diluted with water (20 mL) and extracted with DCM (3×40 mL). The combined organic layers were washed with brine (2×30 mL), dried over Na₂SO₄, filtered and concentrated in vacuo to give the title compound (1.90 g, crude) as yellow oil. ¹H NMR (400 MHz, CDCl₃) δ 5.23-5.11 (m, 1H), 4.79-4.66 (m, 1H), 4.34 (t, J=6.0 Hz, 2H), 4.09-4.04 (m, 1H), 3.42 (t, J=6.0 Hz, 2H), 3.02 (s, 3H), 2.51-2.26 (m, 4H), 2.00 (q, J=6.0 Hz, 2H), 1.44 (s, 9H).

### Step 4—Tert-butyl N-[3-[3-(methylamino)propoxy]cyclobutyl]carbamate

A mixture of 3-[3-(tert-butoxycarbonylamino)cyclobutoxy]propyl methanesulfonate (1.90 g, 5.87 mmol) and MeNH₂/EtOH (5.87 mmol, 10 mL, 30% solution) was stirred at 70° C. for 12 hrs in a sealed tube (15 psi). On completion, the reaction mixture was concentrated in vacuo to give the title compound (1.80 g, crude) as yellow oil and used for next step directly. LC-MS (ESI⁺) m/z 259.0 (M+H)⁺.

### Step 5—Benzyl N-[3-[3-(tert-butoxycarbonylamino)cyclobutoxy]propyl]-N-methyl-carbamate

To a solution of tert-butyl N-[3-[3-(methylamino)propoxy]cyclobutyl]carbamate (1.80 g, 6.97 mmol) and K₂CO₃ (1.93 g, 13.9 mmol) in a mixed solvents of THF (15 mL) and water (5 mL) was added CbzCl (1.78 g, 10.4 mmol). The reaction mixture was stirred at 20° C. for 2 hrs. On completion, the reaction mixture was diluted with water (30 mL) and extracted with EA (3×50 mL). The combined organic layers were washed with brine (2×30 mL), dried over Na₂SO₄, filtered and concentrated in vacuo. The residue was purified by silica gel chromatography (PE:EA=5:1) to give the title compound (1.30 g, 41% yield) as yellow oil. ¹H NMR (400 MHz, CDCl₃) δ 7.39-7.29 (m, 5H), 5.13 (s, 2H), 4.79-4.58 (m, 1H), 4.32-4.14 (m, 1H), 4.07-3.92 (m, 1H), 3.42-3.21 (m, 4H), 2.94 (s, 3H), 2.39-2.23 (m, 2H), 2.14-2.05 (m, 2H), 1.87-1.72 (m, 2H), 1.45 (s, 9H).

### Step 6—Benzyl N-[3-(3-aminocyclobutoxy)propyl]-N-methyl-carbamate

To a solution of benzyl N-[3-[3-(tert-butoxycarbonylamino)cyclobutoxy]propyl]-N-methyl-carbamate (1.60 g, 4.08 mmol) in DCM (20 mL) was added HCl/dioxane (4 M, 15 mL). The reaction mixture was stirred at 20° C. for 1 hour. On completion, the reaction mixture was concentrated in vacuo to give the title compound (1.30 g, 96% yield, HCl salt) as yellow semisolid. LC-MS (ESI⁺) m/z 293.2 (M+H)⁺.

### N-[6-(difluoromethyl)-2-(4-formylcyclohexyl)indazol-5-yl]-6-(trifluoromethyl) pyridine-2-carboxamide (Intermediate ALU)

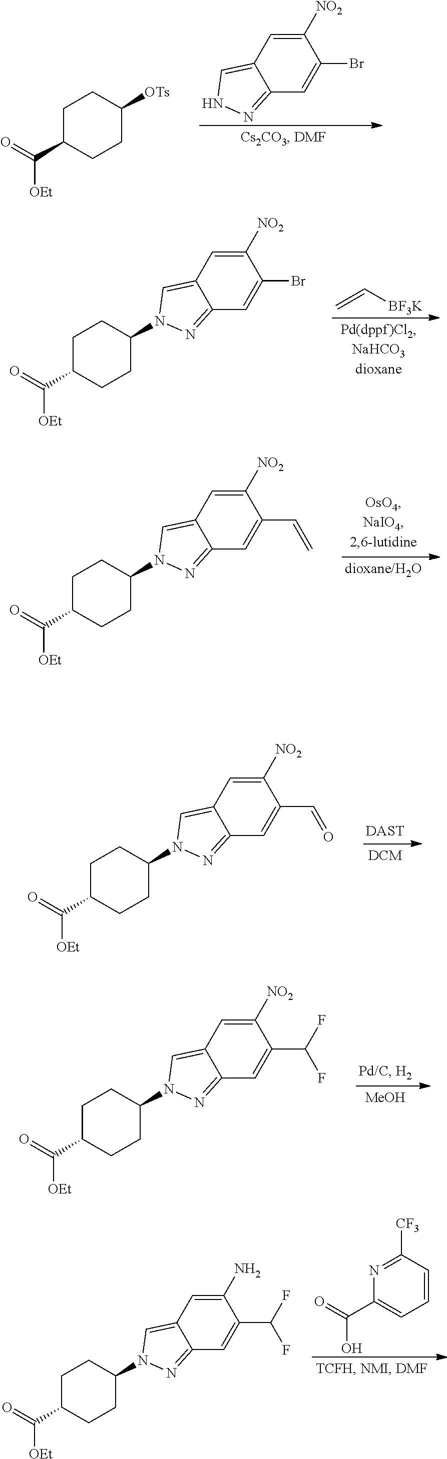

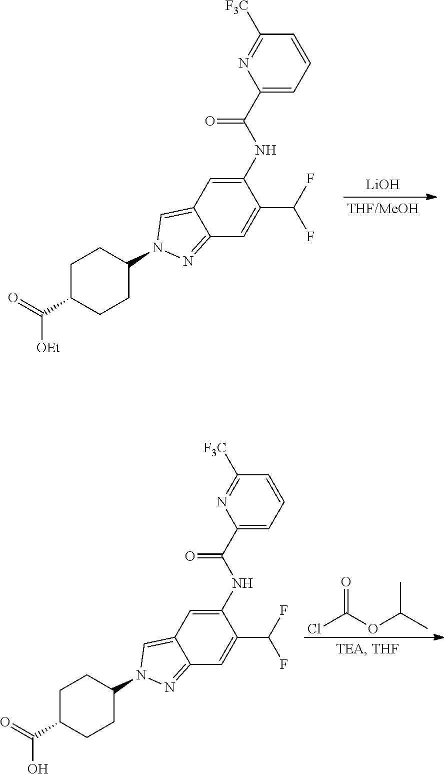

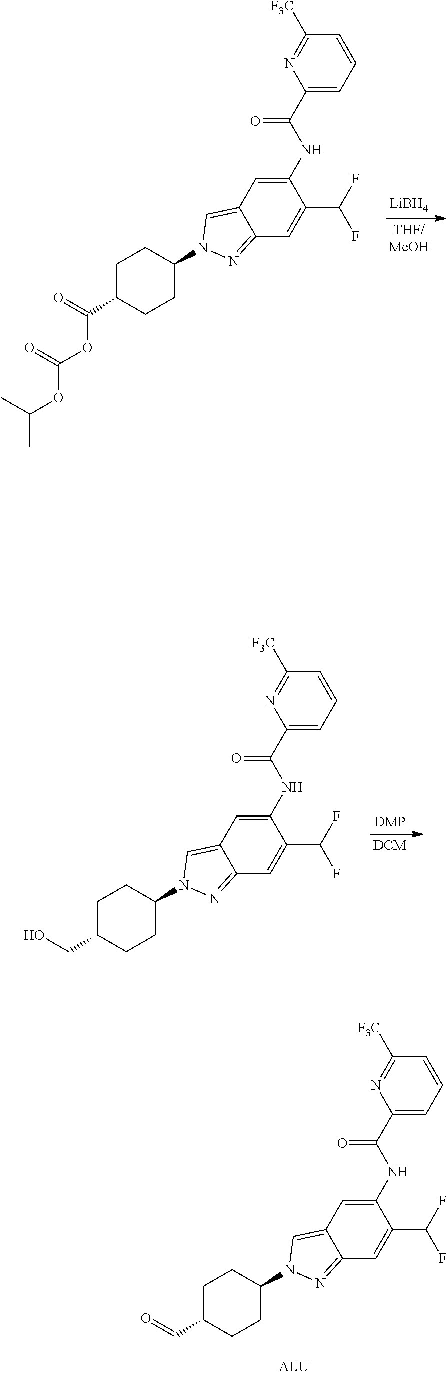

### Step 1—Ethyl 4-(6-bromo-5-nitro-indazol-2-yl)cyclohexanecarboxylate

To a solution of 6-bromo-5-nitro-2H-indazole (8.30 g, 34.3 mmol, CAS #1351813-02-9) and ethyl 4-(p-tolylsulfonyloxy)cyclohexanecarboxylate (22.4 g, 68.6 mmol, Intermediate AGK) in DMF (100 mL) was added Cs₂CO₃ (22.4 g, 68.6 mmol). The mixture was stirred at 80° C. for 24 hrs. On completion, the reaction mixture was concentrated in vacuo to remove solvent. Then to the mixture was added 100 mL water and extracted with EA 150 mL (3×50 mL). The combined organic layers were washed with 100 mL brine, dried over Na₂SO₄, filtered and concentrated in vacuo to give a residue. The residue was purified by flash silica gel chromatography (PE:EA 2:1) and then the residue was purified by pre-HPLC (column: Phenomenex Synergi Max-RP 250*50 mm*10 um; mobile phase: [water (0.1% TFA)-ACN]; B %: 45ACN %-75ACN %, 29 min) to give the title compound (1.60 g, 40% yield) as yellow solid. ¹H NMR (400 MHz, DMSO-d₆) δ 8.60 (s, 1H), 8.45 (s, 1H), 8.34 (s, 1H), 4.91-4.71 (m, 1H), 4.08 (q, J=7.2 Hz, 2H), 2.47-2.40 (m, 1H), 2.10-1.89 (m, 6H), 1.74-1.55 (m, 2H), 1.20 (t, J=7.2 Hz, 3H).

### Step 2—2-(2,6-Dioxo-3-piperidyl)-4-[[(2R)-morpholin-2-yl]methylamino]isoindoline-1,3-dione

To a solution of ethyl 4-(6-bromo-5-nitro-indazol-2-yl)cyclohexanecarboxylate (1.60 g, 4.04 mmol) and potassium; trifluoro(vinyl)boranuide (1.62 g, 12.1 mmol) and potassium; trifluoro(vinyl)boranuide (1.62 g, 12.1 mmol) in dioxane (100 mL) was added Pd(dppf)Cl₂ (329 mg, 403 umol) and NaHCO₃ (2 M, 4.04 mL). The mixture was stirred at 90° C. for 6 hrs. On completion, the mixture was filtered with celite and concentrated in vacuo. The crude product was purified by flash silica gel chromatography (PE:EA 1:1) to give the title compound (1.39 g, 50% yield) as yellow solid. LC-MS (ESI⁺) m/z 344.1 (M+1)⁺.

### Step 3-Ethyl 4-(6-formyl-5-nitro-indazol-2-yl)cyclohexanecarboxylate

A mixture of ethyl 4-(5-nitro-6-vinyl-indazol-2-yl)cyclohexanecarboxylate (1.39 g, 4.20 mmol), NaIO₄ (3.74 g, 17.4 mmol), OSO₄ (33.3 mg, 131 umol) and 2,6-dimethylpyridine (936 mg, 8.74 mmol, 1.02 mL) in a mixed solvents of dioxane (20 mL) and H₂O (20 mL) was stirred at 0° C. for 1 hour. On completion, the mixture was added 10 mL Na₂S₂O₄ and extracted with DCM 150 mL (3×50 mL). The combined organic layers were washed with 100 mL brine, dried over Na₂SO₄, filtered and concentrated in vacuo to give the title compound (1.50 g, 80% yield) as yellow solid. LC-MS (ESI⁺) m/z 346.0 (M+1)⁺.

### Step 4—Ehyl 4-[6-(difluoromethyl)-5-nitro-indazol-2-yl]cyclohexanecarboxylate

To a solution of ethyl 4-(6-formyl-5-nitro-indazol-2-yl)cyclohexanecarboxylate (1.50 g, 4.34 mmol) in DCM (80 mL) was added DAST (1.75 g, 10.8 mmol). The mixture was stirred at 0° C. for 2 hrs. On completion, to the mixture was added 2 mL H₂O slowly at 0° C., then the solution was concentrated in vacuo. The crude product was purified by reverse phase HPLC (0.1% TFA condition) to give the title compound (900 mg, 56% yield) as yellow solid. ¹H NMR (400 MHz, DMSO-d₆) δ 8.87 (d, J=5.2 Hz, 2H), 8.07 (s, 1H), 7.73-7.37 (m, 1H), 4.71-4.66 (m, 1H), 4.09 (q, J=7.2 Hz, 2H), 2.24-2.15 (m, 2H), 2.13-1.92 (m, 4H), 1.61 (dq, J=3.1, 12.8 Hz, 2H), 1.20 (t, J=7.2 Hz, 3H); LC-MS (ESI⁺) m/z 368.1 (M+1)⁺.

### Step 5—Ethyl 4-[5-amino-6-(difluoromethyl)indazol-2-yl]cyclohexanecarboxylate

To a solution of ethyl 4-[6-(difluoromethyl)-5-nitro-indazol-2-yl]cyclohexanecarboxylate (900 mg, 2.45 mmol) in THF (20 mL) was added Pd/C (500 mg, 10 wt %). The mixture was stirred at 15° C. for 2 hrs under H₂ (15 psi). On completion, the mixture was filtered through celite and concentrated in vacuo to give the title compound (800 mg, 96% yield) as yellow oil. ¹H NMR (400 MHz, DMSO-d₆) δ 8.07 (s, 1H), 7.65 (s, 1H), 7.23-6.91 (m, 1H), 6.84 (s, 1H), 4.78 (s, 2H), 4.48-4.32 (m, 1H), 3.62 (s, 3H), 2.48-2.40 (m, 1H), 2.17-2.02 (m, 4H), 1.99-1.86 (m, 2H), 1.64-1.50 (m, 2H).

### Step 6—Ethyl 4-[6-(difluoromethyl)-5-[[6-(trifluoromethyl)pyridine-2-carbonyl]amino]indazol-2-yl]cyclohexanecarboxylate

To a solution of 6-(trifluoromethyl)pyridine-2-carboxylic acid (679 mg, 3.56 mmol) and ethyl 4-[5-amino-6-(difluoromethyl)indazol-2-yl]cyclohexanecarboxylate (800 mg, 2.37 mmol) in DMF (20 mL) was added HATU (1.35 g, 3.56 mmol) and DIPEA (919 mg, 7.11 mmol). The mixture was stirred at 70° C. for 2 hrs. On completion, the mixture was added 0.5 mL water, the residue was purified by prep-HPLC (column: Phenomenex luna C18 250*50 mm*10 um; mobile phase: [water (0.1% TFA)-ACN]; B %: 68%-98%, 9 min) to give the title compound (750 mg, 59% yield) as light yellow solid. ¹H NMR (400 MHz, DMSO-d₆) δ 10.50 (s, 1H), 8.56 (s, 1H), 8.49 (s, 1H), 8.48-8.38 (m, 2H), 8.23 (dd, J=1.2, 7.6 Hz, 1H), 8.00 (s, 1H), 7.43-7.09 (m, 1H), 4.65-4.52 (m, 1H), 4.10 (q, J=7.2 Hz, 2H), 2.26-2.16 (m, 2H), 2.12-1.95 (m, 4H), 1.69-1.56 (m, 2H), 1.21 (t, J=7.2 Hz, 3H); LC-MS (ESI+) m/z 511.1 (M+1)⁺.

### Step 7—Ethyl 4-[6-(difluoromethyl)-5-[[6-(trifluoromethyl)pyridine-2-carbonyl]amino]indazol-2-yl]cyclohexane carboxylic acid

To a solution of ethyl 4-[6-(difluoromethyl)-5-[[6-(trifluoromethyl)pyridine-2-carbonyl]amino]indazol-2-yl]cyclohexanecarboxylate (300 mg, 587 umol) in THF (1 mL) and MeOH (0.1 mL) and H₂O (1 mL) was added LiOH·H₂O (123 mg, 2.94 mmol). The mixture was stirred at 15° C. for 2 hrs. On completion, the mixture was concentrated in vacuo and M HCl was added to the mixture until the pH=5-6 then the mixture was filtered. The filtered cake was dried in vacuo to give the title compound (250 mg, 88% yield) as yellow solid. LC-MS (ESI+) m/z 483.2 (M+1)⁺.

### Step 8—Isopropoxycarbonyl 4-[6-(difluoromethyl)-5-[[6-(trifluoromethyl)pyridine-2-carbonyl]amino]indazol-2-yl]cyclohexanecarboxylate

To a solution of 4-[6-(difluoromethyl)-5-[[6-(trifluoromethyl)pyridine-2-carbonyl]amino]indazol-2-yl]cyclohexanecarboxylic acid (100 mg, 207 umol) in THF (1 mL) was added Et₃N (83.9 mg, 829 umol) and isopropyl carbonochloridate (63.5 mg, 518 umol). The mixture was stirred at 0° C. for 2 hrs. On completion, the mixture was filtered and the cake was washed with THF (3×5 mL). The organic phase was concentrated in vacuo to give the title compound (100 mg, 85% yield) as yellow oil. LC-MS (ESI+) m/z 569.0 (M+1)⁺.

### Step 9—N-[6-(difluoromethyl)-2-[4-(hydroxymethyl)cyclohexyl]indazol-5-yl]-6-(trifluoromethyl) pyridine-2-carboxamide

To a solution of isopropoxycarbonyl 4-[6-(difluoromethyl)-5-[[6-(trifluoromethyl)pyridine-2-carbonyl]amino]indazol-2-yl]cyclohexanecarboxylate (100 mg, 175 umol) in THF (10 mL) and H₂O (1 mL) was added LiBH₄ (23.0 mg, 1.06 mmol). The mixture was stirred at 0° C. for 2 hrs. The mixture was added 10 mL NH₄Cl and extracted with EA (3×50 mL). The combined organic layers were washed with 50 mL brine, dried over Na₂SO₄, filtered and concentrated in vacuo to give the title compound (80.0 mg, 97% yield) as yellow solid. LC-MS (ESI+) m/z 469.0 (M+1)⁺.

### Step 10—N-[6-(difluoromethyl)-2-(4-formylcyclohexyl)indazol-5-yl]-6-(trifluoromethyl) pyridine-2-carboxamide

To a solution of N-[6-(difluoromethyl)-2-[4-(hydroxymethyl)cyclohexyl]indazol-5-yl]-6-(trifluoro methyl)pyridine-2-carboxamide (80.0 mg, 170 umol) in DCM (10 mL) was added DMP (108 mg, 256 umol). The mixture was stirred at 15° C. for 2 hrs. On completion, the mixture was added 10 mL sat. Na₂S₂O₃ and 10 mL sat. NaHCO₃ and extracted with DCM 150 mL (3×50 mL). The combined organic layers were washed with 100 mL brine, dried over Na₂SO₄, filtered and the organic phase was concentrated in vacuo to give the title compound (80.0 mg, 95% yield) as yellow solid. LC-MS (ESI+) m/z 467.1 (M+1)⁺.

### 4-[6-(1-hydroxy-1-methyl-ethyl)-5-[[6-(trifluoromethyl)pyridine-2-carbonyl]amino]indazol-2-yl]cyclohexanecarboxylic acid (Intermediate AMY)

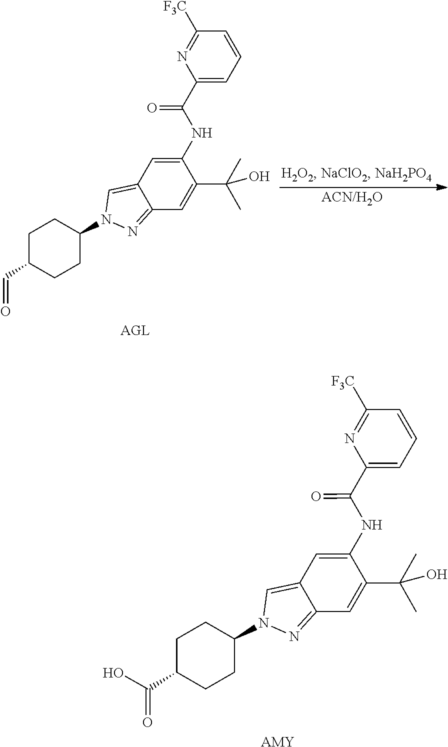

To a solution of N-[2-(4-formylcyclohexyl)-6-(1-hydroxy-1-methyl-ethyl)indazol-5-yl]-6-(trifluoro methyl)pyridine-2-carboxamide (200 mg, 421 umol, Intermediate AGL) and NaH₂PO₄ (252 mg, 2.11 mmol) in ACN (6 mL) was added H₂O₂(95.5 mg, 843 umol, 81 μL, 30% solution) dropwise at 0° C. Then sodium chlorite (266 mg, 2.95 mmol) in H₂O (3 mL) was added to the mixture at 0° C. The reaction mixture was stirred at 25° C. for 1 hour. On completion, the reaction mixture was diluted with ACN (20 mL) and the reaction was quenched with sat.Na₂SO₃ (20 mL) at 0° C. The reaction mixture was extracted with ACN (3×10 mL). The combined organic layers were concentrated in vacuo to give the title compound (200 mg, 96% yield) as a yellow solid. LC-MS (ESI+) m/z 491.1 (M+1)⁺.

### 3H-[1,2,3]triazolo[4,5-b]pyridin-3-yl pyrazolo[1,5-a]pyrimidine-3-carboxylate (Intermediate BIJ)

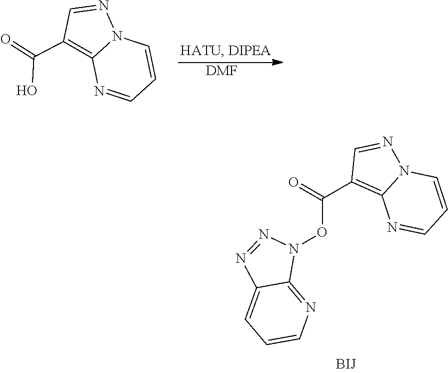

To a solution of pyrazolo[1,5-a]pyrimidine-3-carboxylic acid (1 g, 6.13 mmol, CAS #25940-35-6) in DMF (20 mL) was added HATU (2.33 g, 6.13 mmol) and DIPEA (1.58 g, 12.2 mmol). Then the reaction mixture was stirred at 40° C. for 2 hours. On completion, the reaction mixture was filtered. The filter cake was washed with DCM (2×4 mL) and dried in vacuo to give the title compound (1.10 g, 68% yield) as an off-white solid. LC-MS (ESI⁺) m/z 282.0 (M+H)⁺.

### N-[3-(difluoromethyl)-1-[4-(hydroxymethyl)cyclohexyl]pyrazol-4-yl]pyrazolo[1,5-a]pyrimidine-3-carboxamide (Intermediate BIK)

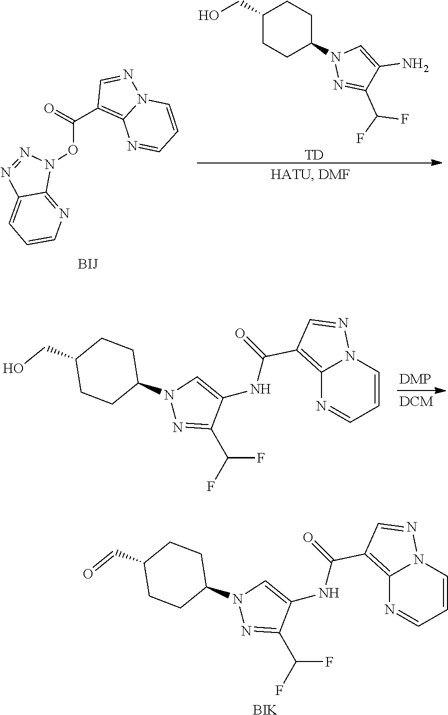 [SMILES: O=C(On1nnc2cccnc21)c1cnn2cccnc12 ; Nc1cn([C@H]2CC[C@H](CO)CC2)nc1C(F)F ; "TD" ; O=C(Nc1cn([C@H]2CC[C@H](CO)CC2)nc1C(F)F)c1cnn2cccnc12 ; O=C[C@H]1CC[C@H](n2cc(NC(=O)c3cnn4cccnc34)c(C(F)F)n2)CC1 ; "BIK"]

### Step 1—N-[3-(difluoromethyl)-1-[4-(hydroxymethyl)cyclohexyl]pyrazol-4-yl] pyrazolo[1,5-a] pyrimidine-3-carboxamide

To a solution of [4-[4-amino-3-(difluoromethyl)pyrazol-1-yl]cyclohexyl]methanol (468 mg, 1.91 mmol, Intermediate TD) in DMF (15 mL) was added triazolo[4,5-b]pyridin-3-yl pyrazolo[1,5-a]pyrimidine-3-carboxylate (536 mg, 1.91 mmol, Intermediate BIJ). The reaction mixture was stirred at 40° C. for 5 hours. On completion, the reaction was quenched with sat. NH₄Cl (30 mL) and extracted with DCM (3×100 mL). The combined organic layers were washed with brine (50 mL), dried with anhydrous Na₂SO₄ and filtered. The filtrate was concentrated in vacuo. The residue was purified by reverse phase (0.1% FA) to give the title compound (400 mg, 53% yield) as a white solid. LC-MS (ESI⁺) m/z 391.2 (M+H)⁺.

### Step 2—N-[3-(difluoromethyl)-1-[4-(hydroxymethyl)cyclohexyl]pyrazol-4-yl]pyrazolo[1,5-a]pyrimidine-3-carboxamide

To a solution of N-[3-(difluoromethyl)-1-[4-(hydroxymethyl)cyclohexyl]pyrazol-4-yl]pyrazolo [1,5-a]pyrimidine-3-carboxamide (200 mg, 512 umol) in DCM (10 mL) was added DMP (325 mg, 768 umol). The reaction mixture was stirred at 25° C. for 1 hour. On completion, the reaction was quenched with sat. NaS₂O₃ (30 mL) and extracted with DCM (2×30 mL). The combined organic layers were washed with brine (20 mL), dried with anhydrous Na₂SO₄ and filtered. The filtrate was concentrated in vacuo to give the title compound (198 mg, 509 umol, 99% yield) as a white solid. LC-MS (ESI⁺) m/z 389.2 (M+H)⁺.

### N-[3-(difluoromethyl)-1-(4-formylcyclohexyl) pyrazol-4 yl] pyrazolo [1,5-a]pyrimidine-3-carboxamide (Intermediate BHI)

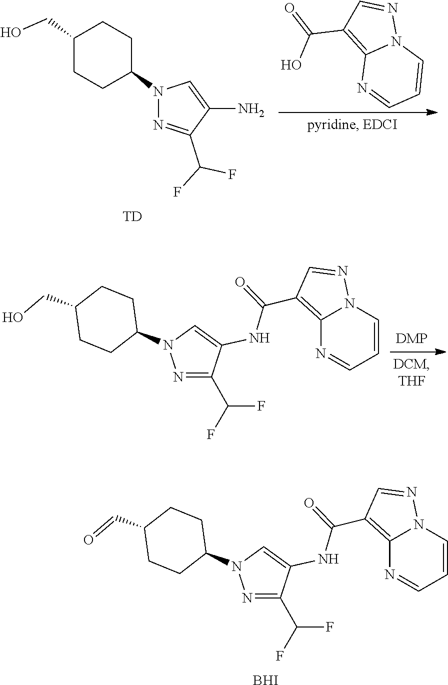

### Step 1—N-[3-(difluoromethyl)-1-[4-(hydroxymethyl) cyclohexyl]pyrazol-4-yl]pyrazolo[1,5-a]pyrimidine-3-carboxamide

To a mixture of pyrazolo[1,5-a]pyrimidine-3-carboxylic acid (0.13 g, 796 umol, CAS #25940-35-6) and [4-[4-amino-3-(difluoromethyl) pyrazol-1-yl]cyclohexyl]methanol (195 mg, 796 umol, Intermediate TD) in pyridine (2 mL) was added EDCI (183.32 mg, 956.27 umol) in one portion at 25° C. under N₂ and the mixture was stirred at 25° C. for 3 hrs. On completion, the mixture was concentrated in vacuo to give the residue. The residue was purified by column chromatography (petroleum ether/ethyl acetate=1/0 to DCM/MeOH=3/1) to give title compound (200 mg, 64% yield) as a yellow solid. ¹H NMR (400 MHz, CDCl₃) δ=10.21 (s, 1H), 8.82 (dd, J=6.8, 1.6 Hz, 1H), 8.77-8.71 (m, 2H), 8.37 (s, 1H), 7.06 (dd, J=6.8, 4.0 Hz, 1H), 6.70-6.99 (m, 1H), 4.12-4.08 (m, 1H), 3.55 (t, J=5.6 Hz, 2H), 2.29-2.22 (m, 2H), 2.06-1.98 (m, 2H), 1.85-1.80 (m, 2H), 1.41-1.38 (m, 1H), 1.27-1.14 (m, 3H).

### Step 2—N-[3-(difluoromethyl)-1-(4-formylcyclohexyl) pyrazol-4-yl]pyrazolo [1,5-a]pyrimidine-3-carboxamide

To a mixture of N-[3-(difluoromethyl)-1-[4-(hydroxymethyl)cyclohexyl]pyrazol-4-yl]pyrazolo [1,5-a]pyrimidine-3-carboxamide (0.14 g, 358 umol) in DCM (2 mL) and THF (0.5 mL) was added DMP (182 mg, 430 umol) in one portion at 25° C. and the mixture was stirred at 25° C. for 1 h. The reaction mixture was then quenched by addition saturated solution of Na₂S₂O₃ (2 mL), and saturated solution of NaHCO₃ (10 mL), then extracted with DCM (10 mL×2). The combined organic layer was washed with brine (10 mL×2), dried over Na₂SO₄, filtered and concentrated in vacuo to give crude title compound (139 mg, 99% yield) as yellow solid. ¹H NMR (400 MHz, CDCl₃) δ=10.22 (s, 1H), 9.71 (d, J=0.8 Hz, 1H), 8.83 (dd, J=7.2, 1.6 Hz, 1H), 8.76-8.73 (m, 2H), 8.39 (s, 1H), 7.07 (dd, J=6.8, 4.0 Hz, 1H), 6.98-6.69 (m, 1H), 4.15-4.05 (m, 1H), 2.34 (dd, J=12.4, 2.8 Hz, 2H), 2.27-2.20 (m, 2H), 1.96-1.82 (m, 3H), 1.57-1.50 (m, 2H).

### N-[3-(difluoromethyl)-1-(3-formylcyclobutyl)pyrazol-4-yl]-5-[(1R,4R)-2-oxa-5-azabicyclo[2.2.1]heptan-5-yl]pyrazolo[1,5-a]pyrimidine-3-carboxamide (Intermediate BWA)

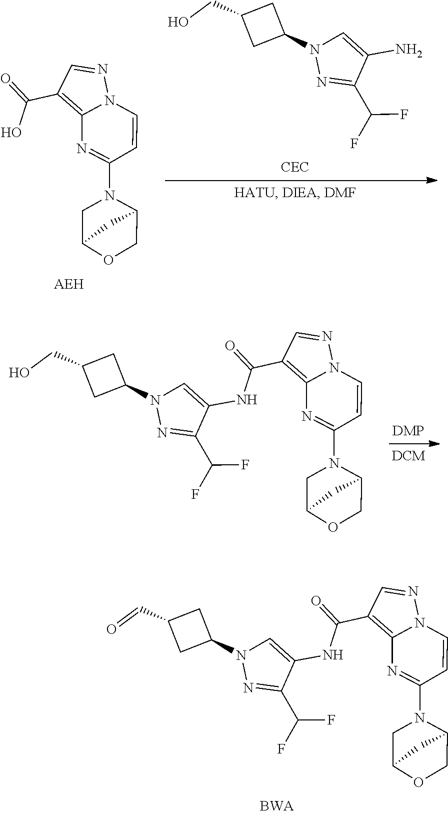 [SMILES: O=C(O)c1cnn2ccc(N3C[C@H]4C[C@@H]3CO4)nc12 ; Nc1cn([C@H]2C[C@H](CO)C2)nc1C(F)F ; O=C(Nc1cn([C@H]2C[C@H](CO)C2)nc1C(F)F)c1cnn2ccc(N3C[C@H]4C[C@@H]3CO4)nc12 ; O=C[C@H]1C[C@H](n2cc(NC(=O)c3cnn4ccc(N5C[C@H]6C[C@@H]5CO6)nc34)c(C(F)F)n2)C1]

### Step 1—Methyl 5-[[4-(benzyloxymethyl)cyclohexanecarbonyl]amino]-2-bromo-4-iodo-benzoate

To a solution of 5-[(1R,4R)-2-oxa-5-azabicyclo[2.2.1]heptan-5-yl]pyrazolo[1,5-a]pyrimidine-3-carboxylic acid (335 mg, 1.29 mmol, Intermediate AEH) and DIEA (357 mg, 2.76 mmol) in DMF (4 mL) was added HATU (420 mg, 1.10 mmol). The reaction mixture was stirred at 25° C. for 1 hour. Then [3-[4-amino-3-(difluoromethyl) pyrazol-1-yl]cyclobutyl]methanol (200 mg, 920 umol, Intermediate CEC) was added and the reaction mixture was stirred at 25° C. for 14 hours. On completion, the reaction mixture was poured into saturated aqueous sodium bicarbonate (40 mL). The mixture was extracted with ethyl acetate (30 mL×4). The combined organic layers were dried over sodium sulfate and then concentrated under vacuum to get the residue. The residue was purified by prep-TLC (10% methanol in dichloromethane) to give the title compound (211 mg, 50% yield) as white solid. ¹H NMR (400 MHz, DMSO-d₆) δ 9.50 (d, J=5.2 Hz, 1H), 8.78 (d, J=7.6 Hz, 1H), 8.40 (d, J=4.8 Hz, 1H), 8.26 (d, J=5.6 Hz, 1H), 7.32-6.98 (m, 1H), 6.90-6.37 (m, 1H), 5.35-5.03 (m, 1H), 5.01-4.90 (m, 1H), 4.77-4.78 (m, 1H), 4.72-4.70 (m, 1H), 3.84-3.71 (m, 2H), 3.67-3.57 (m, 2H), 3.56-3.49 (m, 2H), 3.43-3.45 (m, 1H), 2.43-2.34 (m, 1H), 2.30-2.19 (m, 2H), 2.06-1.87 (m, 2H), 1.32-1.20 (m, 1H). LC-MS (ESI⁺) m/z 460.3 (M+H)⁺.

### Step 2—N-[3-(difluoromethyl)-1-(3-formylcyclobutyl)pyrazol-4-yl]-5-[(1R,4R)-2-oxa-5-azabicyclo[2.2.1]heptan-5-yl]pyrazolo[1,5-a]pyrimidine-3-carboxamide

To a solution of N-[3-(difluoromethyl)-1-[3-(hydroxymethyl)cyclobutyl]pyrazol-4-yl]-5-[(1R,4R)-2-oxa-5-azabicyclo[2.2.1]heptan-5-yl]pyrazolo[1,5-a]pyrimidine-3-carboxamide (80.0 mg, 174 umol) in DCM (3 mL) at 0° C. was added DMP (81.2 mg, 191 umol). The reaction mixture was stirred at 20° C. for 1 hour. On completion, the reaction mixture was partitioned between dichloromethane (30 mL) and NaHCO₃ (10 mL), then diluted with water (10 mL). The organic phase was separated, washed with H₂O (10 mL), dried over anhydrous sodium sulfate, concentrated under reduced pressure to give a residue. The residue was purified by column chromatography (SiO₂, Dichloromethane/Methanol=20/1) to give the title compound (79.0 mg, 72% yield) as white solid. ¹H NMR (400 MHz, CDCl₃) δ 9.92 (s, 1H), 9.62 (s, 1H), 8.51-8.41 (m, 2H), 8.37-8.30 (m, 1H), 6.68-6.93 (m, 1H), 6.13 (d, J=7.6 Hz, 1H), 5.46 (s, 1H), 4.87-4.76 (m, 2H), 4.01-3.94 (m, 2H), 3.39-3.27 (m, 1H), 3.17-2.90 (m, 1H), 2.86-2.80 (m, 3H), 2.15-2.07 (m, 3H), 1.98-1.99 (m, 2H). LC-MS (ESI⁺) m/z 458.3 (M+H)⁺.

### 1-[8-[(1R,4R)-2,5-diazabicyclo[2.2.1]heptan-2-yl]-4-isoquinolyl]hexahydropyrimidine-2,4-dione (Intermediate BWB)

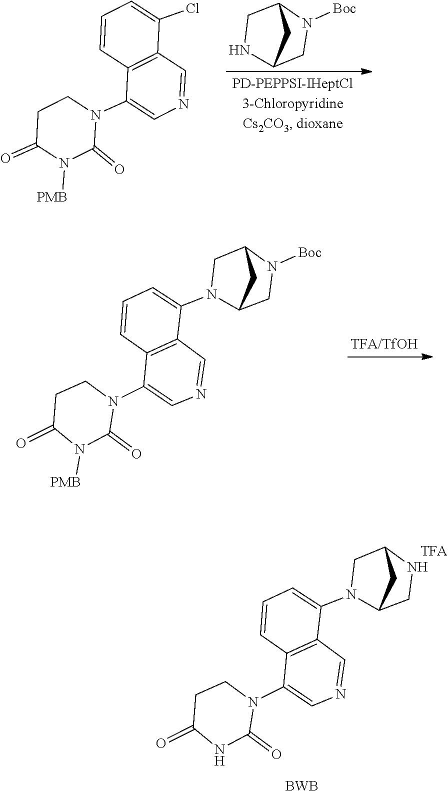

### Step 1—Tert-butyl (1R,4R)-5-[4-[3-[(4-methoxyphenyl)methyl]-2,4-dioxo-hexahydropyrimidin-1-yl]-8-isoquinolyl]-2,5-diazabicyclo[2.2.1]heptane-2-carboxylate

To a mixture of 1-(8-chloro-4-isoquinolyl)-3-[(4-methoxyphenyl) methyl]hexahydropyrimidine-2,4-dione (500 mg, 1.26 mmol, synthesized via Steps 1-2 of Intermediate BSA) and tert-butyl (1R,4R)-2,5-diazabicyclo[2.2.1]heptane-2-carboxylate (275 mg, 1.39 mmol, CAS #134003-84-2) in dioxane (10 mL) was added PD-PEPPSI-IHeptCl 3-Chloropyridine (122 mg, 126 umol) and Cs₂CO₃ (1.23 g, 3.79 mmol). The reaction mixture was then stirred at 80° C. for 12 hours. On completion, the residue was diluted with water (30 mL), then the residue was extracted with EA (3×30 mL). The combined organic layer was dried over Na₂SO₄, filtered and concentrated in vacuo to give a residue. The residue was purified by reverse phase (0.1% FA condition) to give the title compound (236 mg, 33% yield) as yellow solid. ¹H NMR (400 MHz, DMSO-d₆) δ 9.38 (d, J=8.0 Hz, 1H), 8.45 (d, J=6.8 Hz, 1H), 7.62 -7.54 (m, 1H), 7.31-7.18 (m, 3H), 7.03 (t, J=8.4 Hz, 1H), 6.90-6.85 (m, 2H), 4.87-4.77 (m, 2H), 4.55 (d, J=6.0 Hz, 1H), 4.50-4.41 (m, 1H), 4.17-4.01 (m, 1H), 3.92 (ddd, J=5.2, 9.6, 12.0 Hz, 1H), 3.87-3.79 (m, 1H), 3.73 (s, 3H), 3.73-3.68 (m, 1H), 3.63-3.53 (m, 1H), 3.45-3.35 (m, 2H), 3.10 (tdd, J=6.0, 9.6, 16.4 Hz, 1H), 3.00-2.91 (m, 1H), 2.05 (s, 1H), 2.00-1.89 (m, 1H), 1.37 (d, J=5.2 Hz, 9H).

### Step 2—1-[8-[(1R,4R)-2,5-diazabicyclo[2.2.1]heptan-2-yl]-4-isoquinolyl]hexahydropyrimidine-2,4-dione

To a mixture of tert-butyl (1R,4R)-5-[4-[3-[(4-methoxyphenyl)methyl]-2,4-dioxo-hexahydropyrimidin-1-yl]-8-isoquinolyl]-2,5-diazabicyclo[2.2.1]heptane-2-carboxylate (150 mg, 268 umol) in TFA (1 mL) was added TfOH (0.3 mL). The reaction mixture was stirred at 70° C. for 1 hour. On completion, the reaction mixture was concentrated in vacuo to give the title compound (120 mg, 98% yield, TFA). LC-MS (ESI⁺) m/z 338.2 (M+H)⁺.

### 3-[4-(3,6-Diazabicyclo[3.1.1]heptan-3-yl)-3-methyl-2-oxo-benzimidazol-1-yl]piperidine-2,6-dione (Intermediate BWC)

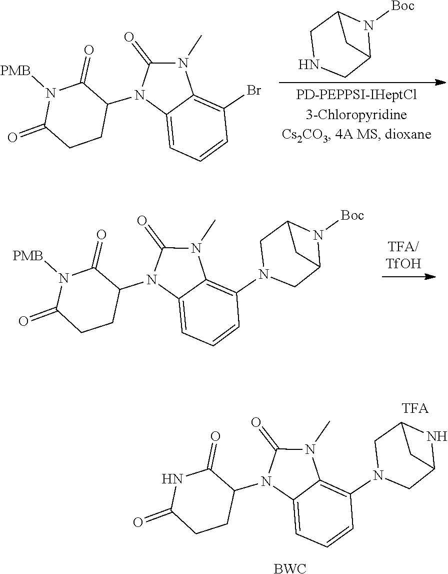

### Step 1—Tert-butyl 3-[1-[1-[(4-methoxyphenyl)methyl]-2,6-dioxo-3-piperidyl]-3-methyl-2-oxo-benzimidazol-4-yl]-3,6-diazabicyclo[3.1.1]heptane-6-carboxylate

To a mixture of 3-(4-bromo-3-methyl-2-oxo-benzimidazol-1-yl)-1-[(4-methoxyphenyl)methyl]piperidine-2,6-dione (1.00 g, 2.18 mmol, synthesized via Steps 1-4 of Intermediate HP) and tert-butyl 3,6-diazabicyclo[3.1.1]heptane-6-carboxylate (432 mg, 2.18 mmol, CAS #869494-16-6) in dioxane (15 mL) was added PD-PEPPSI-IHeptCl 3-Chloropyridine (200 mg, 2.18 mmol) and Cs₂CO₃ (2.13 g, 6.55 mmol) and 4 Å molecular sieves (200 mg, 2.18 mmol). The reaction mixture was stirred at 100° C. for 12 hrs. On completion, the residue was diluted with water (30 mL), then the residue was extracted with EA (3×100 mL). The combined organic layer was dried over Na₂SO₄, filtered and concentrated in vacuo to give a residue. The residue was purified by column chromatography and the residue was purified by reverse phase (0.1% FA condition) to give the title compound (470 mg, 37% yield) as white solid. ¹H NMR (400 MHz, DMSO-d₆) δ 7.21 (d, J=8.4 Hz, 2H), 7.04-6.99 (m, 1H), 6.96-6.93 (m, 1H), 6.86 (d, J=8.4 Hz, 3H), 5.55-5.49 (m, 1H), 4.85-4.73 (m, 2H), 4.14 (d, J=5.6 Hz, 2H), 3.72 (s, 3H), 3.69-3.62 (m, 1H), 3.56 (s, 3H), 3.52-3.42 (m, 1H), 3.23-2.99 (m, 3H), 2.85-2.65 (m, 2H), 2.46-2.35 (m, 1H), 2.08-2.02 (m, 1H), 1.89 (d, J=8.0 Hz, 1H), 1.45 (s, 9H). LC-MS (ESI⁺) m/z 576.3 (M+H)⁺.

### Step 2—3-[4-(3, 6-Diazabicyclo[3.1.1]heptan-3-yl)-3-methyl-2-oxo-benzimidazol-1-yl]piperidine-2,6-dione

To a solution of tert-butyl 3-[1-[1-[(4-methoxyphenyl)methyl]-2,6-dioxo-3-piperidyl]-3-methyl-2-oxo-benzimidazol-4-yl]-3,6-diazabicyclo[3.1.1]heptane-6-carboxylate (100 mg, 173 umol) in TFA (2 mL) was added TfOH (0.4 mL). The reaction mixture was then stirred at 70° C. for 2 hrs. On completion the reaction mixture was concentrated in vacuo to give the title compound (80.0 mg, 98% yield, TFA) as brown liquid. LC-MS (ESI⁺) m/z 356.2 (M+H)⁺.

### Methyl 4-azidocyclohexanecarboxylate (Intermediate BWD)

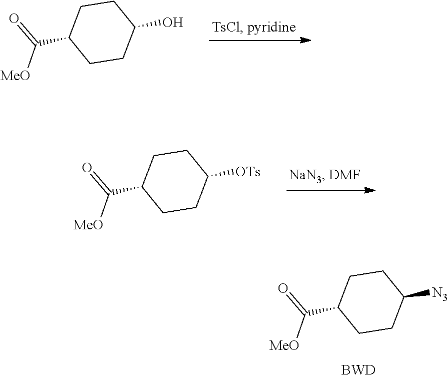

### Step 1—Methyl 4-(p-tolylsulfonyloxy)cyclohexanecarboxylate

To a solution of methyl 4-hydroxycyclohexanecarboxylate (500 mg, 3.16 mmol) in pyridine (2 mL) was added TOSCl (903 mg, 4.74 mmol, CAS #3618-03-9), then the reaction mixture was stirred at 50° C. for 2 hrs. On completion, the reaction mixture was quenched with NaHCO₃ (10 ml), and extracted with EA (2×20 mL). The combined organic phase was washed with brine (2×15 mL), dried with anhydrous Na₂SO₄, filtered and the filtrate was concentrated in vacuo to give the title compound (800 mg, 81% yield) as a colorless oil. ¹H NMR (400 MHz, DMSO-d₆) δ 7.80 (d, J=8.4 Hz, 2H), 7.47 (d, J=8.0 Hz, 2H), 4.70-4.60 (m, 1H), 3.59 (s, 3H), 2.42 (s, 3H), 2.40-2.30 (m, 1H), 1.69-1.51 (m, 8H).

### Step 2—Methyl 4-azidocyclohexanecarboxylate

To a solution of methyl 4-(p-tolylsulfonyloxy)cyclohexanecarboxylate (800 mg, 2.56 mmol) in DMF (10 mL) was added NaN₃ (460 mg, 7.08 mmol), then the reaction mixture was stirred at 80° C. for 16 hrs. On completion, the reaction mixture was diluted with water (50 mL) and extracted with EA (2×20 mL). The combined organic layer was washed with brine (2×20 mL), dried over Na₂SO₄, filtered and the filtrate was concentrated to give the title compound (460 mg, 98% yield) as colorless oil. ¹H NMR (400 MHz, DMSO-d₆) δ 3.59 (s, 3H), 3.48-3.36 (m, 1H), 2.37-2.27 (m, 1H), 1.98-1.86 (m, 4H), 1.49-1.25 (m, 4H).

### 2-Chloro-5-ethynyl-N-isopropyl-pyridin-4-amine (Intermediate BWE)

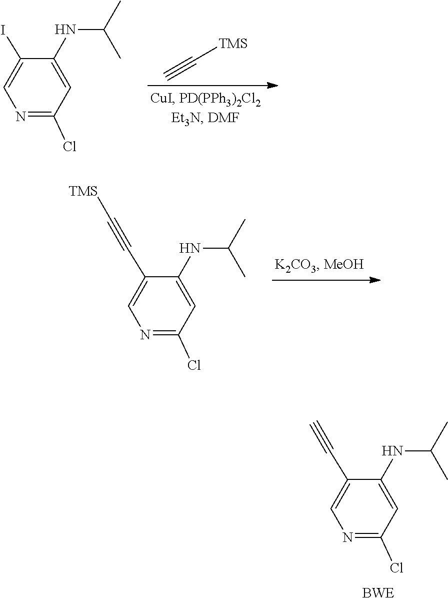

### Step 1—2-Chloro-N-isopropyl-5-(2-trimethylsilylethynyl)pyridin-4-amine

To a solution of 2-chloro-5-iodo-N-isopropyl-pyridin-4-amine (2.00 g, 6.74 mmol, CAS #1447227-69-1) and ethynyl(trimethyl)silane (2.65 g, 26.9 mmol, 3.74 mL, CAS #1066-54-2) in DMF (15 mL) was added CuI (128 mg, 674 umol), TEA (3.41 g, 33.7 mmol, 4.69 mL) and Pd(PPh₃)₂Cl₂ (473 mg, 674 umol). Then the reaction mixture was stirred at 80° C. for 16 hrs under N₂. On completion, the reaction mixture was diluted with water (50 mL) and extracted with EA (2×30 mL). The combined organic layer was washed with brine (2×20 mL) and dried over Na₂SO₄, filtered and the filtrate was concentrated in vacuo to give a residue. The residue was purified by reverse phase (0.1% FA condition) and then column chromatography (SiO₂, PE:EA=50:1 to PE:EA=5:1, PE:EA=3:1, Rf=0.54) to give the title compound (800 mg, 44% yield) as colorless oil. ¹H NMR (400 MHz, DMSO-d₆) δ 8.00 (s, 1H), 6.70 (s, 1H), 5.44 (d, J=8.0 Hz, 1H), 3.90-3.75 (m, 1H), 1.19 (d, J=6.0 Hz, 6H), 0.26 (s, 9H), LC-MS (ESI⁺) m/z 267.4 (M+H)⁺.

### Step 2—2-Chloro-5-ethynyl-N-isopropyl-pyridin-4-amine

To a solution of 2-chloro-N-isopropyl-5-(2-trimethylsilylethynyl)pyridin-4-amine (680 mg, 2.55 mmol) in MeOH (5 mL) was added K₂CO₃ (704 mg, 5.10 mmol), then the reaction mixture was stirred at 25° C. for 2 hrs. On completion, the reaction mixture was filtered and the filtrate was concentrated in vacuo to give the title compound (480 mg, 96% yield) as colorless oil. LC-MS (ESI⁺) m/z 195.1 (M+H)⁺.

### 1-[5-[1-(4-Formylcyclohexyl)triazol-4-yl]-4-(isopropylamino)-2-pyridyl]pyrrolo[2,3-b]pyridine-5-carbonitrile (Intermediate BWF)

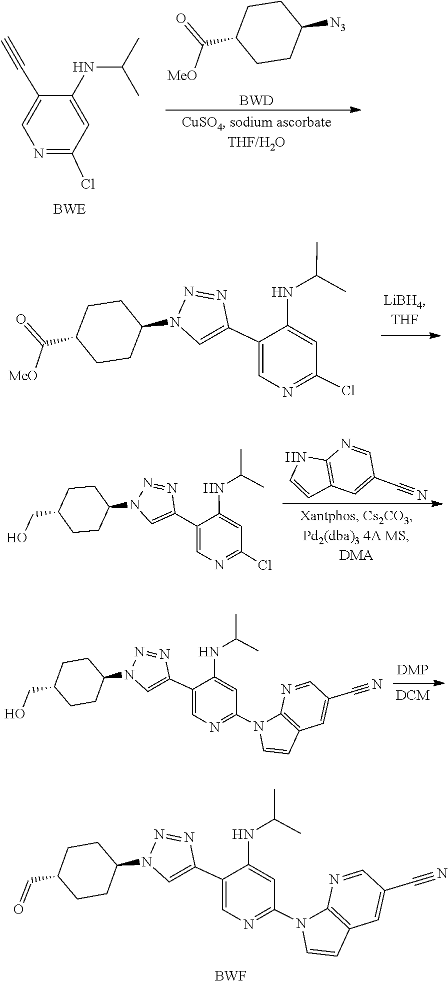

### Step 1—Methyl 4-[4-[6-chloro-4-(isopropylamino)-3-pyridyl]triazol-1-yl]cyclohexanecarboxylate

To a solution of 2-chloro-5-ethynyl-N-isopropyl-pyridin-4-amine (430 mg, 2.21 mmol, Intermediate BWE) and methyl 4-azidocyclohexanecarboxylate (367 mg, 2.01 mmol, Intermediate BWD) in H₂O (3 mL) and THF (3 mL) was added sodium ascorbate (159 mg, 803 umol) and CUSO₄·5H₂O (200 mg, 803 umol). Then the reaction mixture was stirred at 25° C. for 5 hrs. On completion, the reaction mixture was filtered and the filtrate was concentrated in vacuo to give a residue. The residue was diluted with water (30 mL) and extracted with EA (2×30 mL). The combined organic layer was washed with brine (2×20 mL) and dried over Na₂SO₄, filtered and the filtrate was concentrated in vacuo to give a residue. The residue was purified by prep-HPLC (column: YMC Triart C18 250*50 mm*7 um; mobile phase: [water (0.225% FA)-ACN]; B %: 45%-75%, 10 min) to give the title compound (500 mg, 65% yield) as a white solid. ¹H NMR (400 MHz, DMSO-d₆) δ 8.82 (s, 1H), 8.35 (s, 1H), 8.26 (d, J=7.2 Hz, 1H), 6.71 (s, 1H), 4.64-4.52 (m, 1H), 3.84 (dd, J=6.4, 13.2 Hz, 1H), 3.63 (s, 3H), 2.47-2.39 (m, 1H), 2.26-2.18 (m, 2H), 2.13-2.03 (m, 2H), 1.95-1.80 (m, 2H), 1.68-1.55 (m, 2H), 1.23 (d, J=6.4 Hz, 6H), LC-MS (ESI⁺) m/z 378.2 (M+H)⁺.

### Step 2—[4-[4-[6-Chloro-4-(isopropylamino)-3-pyridyl]triazol-1-yl]cyclohexyl]methanol

To a solution of methyl 4-[4-[6-chloro-4-(isopropylamino)-3-pyridyl]triazol-1-yl]cyclohexanecarboxylate (200 mg, 529 umol) in THF (3 mL) was added LiBH₄ (30.0 mg, 1.38 mmol) at 0° C., then the reaction mixture was stirred at 70° C. for 2 hrs. On completion, the reaction mixture was quenched with water (15 mL) and extracted with DCM (2×15 mL). The combined organic layer was washed with brine (2×10 mL) and dried over Na₂SO₄, filtered and the filtrate was concentrated in vacuo to give the title compound (180 mg, 97% yield) as a white solid. LC-MS (ESI⁺) m/z 350.2 (M+H)⁺.

### Step 3—1-[5-[1-[4-(Hydroxymethyl)cyclohexyl]triazol-4-yl]-4-(isopropylamino)-2-pyridyl]pyrrolo [2,3-b]pyridine-5-carbonitrile

To a solution of [4-[4-[6-chloro-4-(isopropylamino)-3-pyridyl]triazol-1-yl]cyclohexyl]methanol (168 mg, 480 umol) and 1H-pyrrolo[2,3-b]pyridine-5-carbonitrile (103 mg, 720 umol, CAS #517918-95-5) in DMA (4 mL) was added Pd₂(dba)₃ (43.9 mg, 48.0 umol), 4 Å molecular sieves (50.0 mg), Xantphos (27.7 mg, 48.0 umol) and Cs₂CO₃ (312 mg, 960 umol) in DMA (4 mL). Then the reaction mixture was stirred at 130° C. for 16 hrs. On completion, the reaction mixture was filtered and the filtrate was concentrated in vacuo to give a residue. The residue was purified by prep-HPLC (column: Phenomenex luna C18 150*25 mm*10 um; mobile phase: [water(0.225% FA)-ACN]; B %: 35%-%, 10 min) to give the title compound (40 mg, 18% yield) as a yellow solid. LC-MS (ESI⁺) m/z 457.2 (M+H)⁺.

### Step 4—1-[5-[1-(4-Formylcyclohexyl)triazol-4-yl]-4-(isopropylamino)-2-pyridyl]pyrrolo[2,3-b]pyridine-5-carbonitrile

To a solution of 1-[5-[1-[4-(hydroxymethyl)cyclohexyl]triazol-4-yl]-4-(isopropylamino)-2-pyridyl]pyrrolo[2,3-b]pyridine-5-carbonitrile (25.0 mg, 54.7 umol) in DCM (2 mL) was added DMP (30.1 mg, 71.1 umol, 22.0 uL) at 0° C., then the reaction mixture was stirred at 25° C. for 5 hrs. On completion, the reaction mixture was quenched with Na₂S₂O₃ (5 mL) and NaHCO₃ (5 mL), then extracted with DCM (2×10 mL). The combined organic phase was washed with NaHCO₃ (8 mL) and brine (2×8 mL), dried with anhydrous Na₂SO₄, filtered and the filtrate was concentrated in vacuo to give the title compound (24 mg, 96% yield) as a yellow solid. LC-MS (ESI⁺) m/z 455.2 (M+H)⁺.

### [4-[5-amino-6-(trifluoromethoxy)indazol-2-yl]cyclohexyl]methanol (Intermediate BWG)

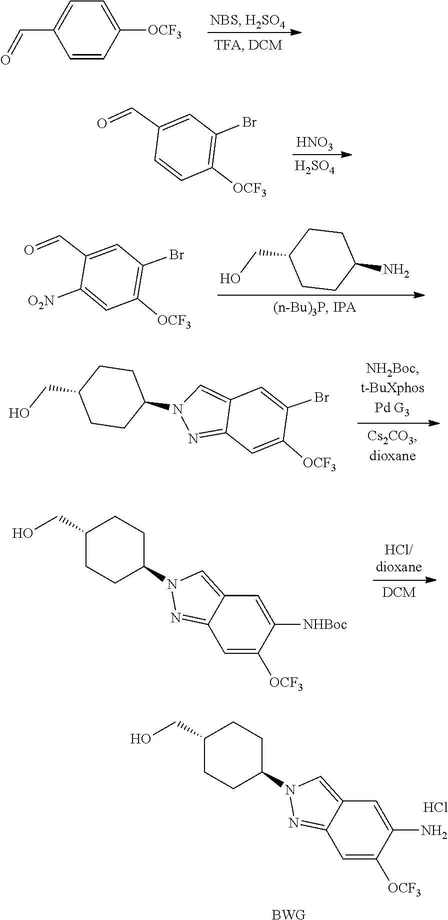

### Step 1—3-bromo-4-(trifluoromethoxy)benzaldehyde

To a solution of 4-(trifluoromethoxy) benzaldehyde (30 g, 157 mmol, CAS #659-68-9) in a mixture of TFA (20 mL), H₂SO₄ (10 mL) and DCM (20 mL) was added NBS (56.1 g, 315 mmol) in portions. The mixture was stirred at 25° C. for 16 hrs. On completion, the residue was diluted with water (150 mL) and extracted with DCM (2×50 mL). The combined organic phase was washed with NaHCO₃ (60 mL) and brine (2×50 mL), dried with anhydrous Na₂SO₄, filtered and the filtrate was concentrated in vacuo to give the title compound (20.7 mg, 49% yield) as yellow solid. ¹H NMR (400 MHz, CDCl₃) δ 10.02 (s, 1H), 7.95 (d, J=8.4 Hz, 2H), 7.39 (s, 1H).

### Step 2—5-bromo-2-nitro-4-(trifluoromethoxy)benzaldehyde

To a solution of 3-bromo-4-(trifluoromethoxy)benzaldehyde (20 g, 74.3 mmol) in H₂SO₄ (150 mL) was added HNO₃(14.4 g, 153 mmol) at 0° C. The mixture was then stirred at 0° C. for 12 hrs. On completion, the reaction mixture was quenched with water (0.05 mL) and concentrated in vacuo. The residue was purified by column chromatography (SiO₂, PE:EA=50:1 to PE:EA=5:1, PE:EA=3:1, P1:Rf=0.3) to give the title compound (5 g, 21% yield) as yellow oil. ¹H NMR (400 MHz, CDCl₃) δ 10.43 (s, 1H), 8.28 (s, 1H), 8.08 (d, J=1.2 Hz, 1H).

### Step 3—[4-[5-bromo-6-(trifluoromethoxy)indazol-2-yl]cyclohexyl]methanol

A mixture of 5-bromo-2-nitro-4-(trifluoromethoxy)benzaldehyde (4.5 g, 14.33 mmol) and (4-aminocyclohexyl)methanol(1.85 g, 14.3 mmol, CAS #1467-84-1) in IPA (30 mL) was stirred at 80° C. for 2 hrs under nitrogen atmosphere. Then the reaction mixture was cool to 20° C., followed by addition of tributylphosphane (8.70 g, 42.9 mmol). The mixture was then stirred at 80° C. for 12 hrs under N₂ atmosphere. On completion, the residue was concentrated in vacuo and the residue was purified by reverse phase (0.1% FA condition) to give the title compound (3 g, 53% yield) as yellow solid. ¹H NMR (400 MHz, DMSO-d₆) δ 8.51 (s, 1H), 8.21 (s, 1H), 7.80 (s, 1H), 4.48 (ddd, J=4.0, 7.8, 11.2 Hz, 1H), 3.28 (d, J=6.4 Hz, 2H), 2.12 (d, J=9.2 Hz, 2H), 1.99-1.79 (m, 4H), 1.67-1.28 (m, 2H), 1.21-1.05 (m, 2H).

### Step 4—Tert-butyl N-[2-[4-(hydroxymethyl)cyclohexyl]-6-(trifluoromethoxy)indazol-5-yl]carbamate

A mixture of [4-[5-bromo-6-(trifluoromethoxy)indazol-2-yl]cyclohexyl]methanol (500 mg, 1.27 mmol), NH₂Boc (163 mg, 1.40 mmol), Cs₂CO₃ (1.24 g, 3.81 mmol) and [2-(2-aminophenyl) phenyl]-methylsulfonyloxy-palladium; ditertbutyl-[2-(2,4,6-triisopropylphenyl)phenyl]phosphane (101 mg, 127 umol) in dioxane (8 mL) was degassed and purged with N₂ for 3 times. Then the mixture was stirred at 80° C. for 24 hrs under N₂ atmosphere. On completion, the residue was purified by reverse phase (0.1% FA condition) to give the title compound (350 mg, 58% yield) as yellow solid. ¹H NMR (400 MHz, CDCl₃) δ 8.33 (s, 1H), 7.88 (s, 1H), 7.60 (s, 1H), 6.75 (d, J=1.2 Hz, 1H), 4.36 (t, J=11.6 Hz, 1H), 3.57 (d, J=6.0 Hz, 2H), 2.32 (d, J=10.4 Hz, 2H), 2.10-1.89 (m, 8H), 1.55 (s, 9H); LC-MS (ESI+) m/z 430.3 (M+H)⁺.

### Step 5—[4-[5-amino-6-(trifluoromethoxy)indazol-2-yl]cyclohexyl]methanol

Tert-butyl N-[2-[4-(hydroxymethyl)cyclohexyl]-6-(trifluoromethoxy)indazol-5-yl]carbamate (300 mg, 698 umol) was added into in HCl/dioxane (6 mL). The mixture was then stirred at 25° C. for 16 hrs. On completion, the mixture was concentrated in vacuo to give the title compound (200 mg, 78% yield) as a brown oil. LC-MS (ESI+) m/z 330.3 (M+H)⁺.

### 5-cyano-N-(2-((1r, 4r)-4-formylcyclohexyl)-6-(trifluoromethoxy)-2H-indazol-5-yl) nicotinamide (Intermediate BWH)

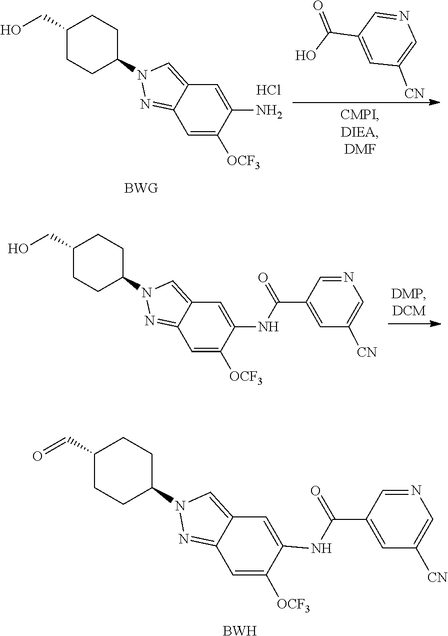

### Step 1—5-cyano-N-[2-[4-(hydroxymethyl)cyclohexyl]-6-(trifluoromethoxy)indazol-5-yl]pyridine-3-carboxamide

To a solution of 5-cyanopyridine-3-carboxylic acid (32.8 mg, 221 umol, CAS #887579-62-6), CMPI (56.5 mg, 221 umol) and DIEA (31.8 mg, 246 umol) in DMF (2 mL) was added [4-[5-amino-6-(trifluoromethoxy) indazol-2-yl]cyclohexyl]methanol (90 mg, 246 umol, Intermediate BWG) and DIEA (31.8 mg, 246 umol) in DMF (2 mL). The mixture was then stirred at 25° C. for 1 hr. On completion, the reaction was diluted with EA (35 mL) and H₂O (20 mL), then extracted with EA (15 mL×2). The combined organic layers were washed with H₂O (15 mL×2), dried over Na₂SO₄, filtered and concentrated under reduced pressure. The residue was purified by column chromatography (SiO₂, PE:EA=5:1 to PE:EA=1:1,PE:EA=1:1, P1:Rf=0.3) to give the title compound (100 mg, 88% yield) as a white solid. LC-MS (ESI+) m/z 460.2 (M+H)⁺.

### Step 2—5-cyano-N-(2-((1r, 4r)-4-formylcyclohexyl)-6-(trifluoromethoxy)-2H-indazol-5-yl) nicotinamide

To a solution of 5-cyano-N-[2-[4-(hydroxymethyl)cyclohexyl]-6-(trifluoromethoxy)indazol-5-yl]pyridine-3-carboxamide (100 mg, 217 umol) in DCM (3 mL) was added DMP (110 mg, 261 umol). The mixture was then stirred at 25° C. for 1 hr. On completion, the reaction mixture was quenched by addition of aqueous Na₂S₂SO₃ (8 mL) and NaHCO₃ (8 mL) at 25° C., and then extracted with DCM 30 mL (15 mL×2). The combined organic layers were dried over anhydrous Na2SO₄ and concentrated in vacuo to give the title compound (80 mg, 80% yield) as a brown solid. LC-MS (ESI+) m/z 457.9 (M+H)⁺.

### 1-(7-Piperazin-1-yl-4-isoquinolyl)hexahydropyrimidine-2,4-dione (Intermediate BWL)

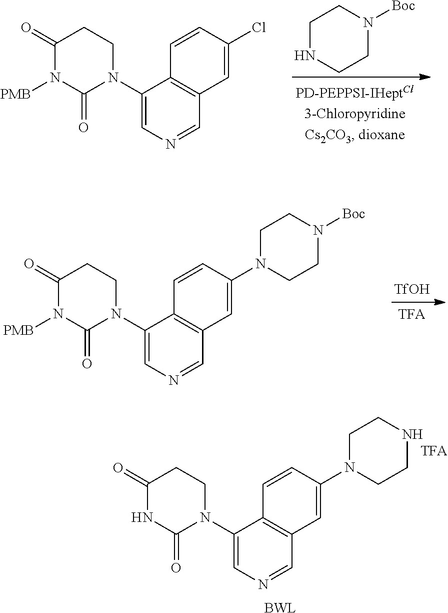

### Step 1—Tert-butyl 4-[4-[3-[(4-methoxyphenyl)methyl]-2,4-dioxo-hexahydropyrimidin-1-yl]-7-isoquinolyl]piperazine-1-carboxylate

To a solution of 1-(7-chloro-4-isoquinolyl)-3-[(4-methoxyphenyl)methyl]hexahydropyrimidine-2,4-dione (500 mg, 1.26 mmol, synthesized via Steps 1-2 of Intermediate BRX), and tert-butyl piperazine-1-carboxylate (258 mg, 1.39 mmol, CAS #143238-38-4) in dioxane (8 mL) was added Cs₂CO₃ (823 mg, 2.53 mmol) and Pd-PEPPSI-IHeptCl 3-Chloropyridine (122 mg, 126 umol). Then the reaction mixture was stirred at 80° C. for 12 hrs under N₂. On completion, the reaction mixture was filtered and the filtrate was concentrated in vacuo to give a residue. The residue was purified by reverse phase (0.1% FA condition) to give the title compound (450 mg, 65% yield) as yellow solid. ¹H NMR (400 MHz, DMSO-d₆) δ 9.09 (s, 1H), 8.30 (s, 1H), 7.77 (d, J=9.2 Hz, 1H), 7.65 (dd, J=2.4, 9.2 Hz, 1H), 7.45 (d, J=2.4 Hz, 1H), 7.25 (d, J=8.8 Hz, 2H), 6.88 (d, J=8.4 Hz, 2H), 4.83 (s, 2H), 3.95-3.84 (m, 1H), 3.81-3.68 (m, 4H), 3.64-3.46 (m, 4H), 3.31-3.26 (m, 2H), 3.18-3.05 (m, 1H), 3.02-2.89 (m, 1H), 2.65-2.52 (m, 2H), 1.44 (s, 9H); LC-MS (ESI⁺) m/z 546.2 (M+H)⁺.

### Step 2—1-(7-Piperazin-1-yl-4-isoquinolyl)hexahydropyrimidine-2,4-dione

To a solution of tert-butyl 4-[4-[3-[(4-methoxyphenyl)methyl]-2,4-dioxo-hexahydropyrimidin-1-yl]-7-isoquinolyl]piperazine-1-carboxylate (80.0 mg, 146 umol) in TfOH (170 mg, 1.13 mmol) was added TFA (770 mg, 6.75 mmol). Then the reaction was stirred at 70° C. for 2 hrs. On completion, the reaction mixture was concentrated in vacuo to give the title compound (60.0 mg, 93% yield, TFA) as yellow oil. LC-MS (ESI⁺) m/z 326.1 (M+H)⁺.

### 3-(4-Bromo-5-methoxy-3-methyl-2-oxo-benzimidazol-1-yl)piperidine-2,6-dione (Intermediate BWM)

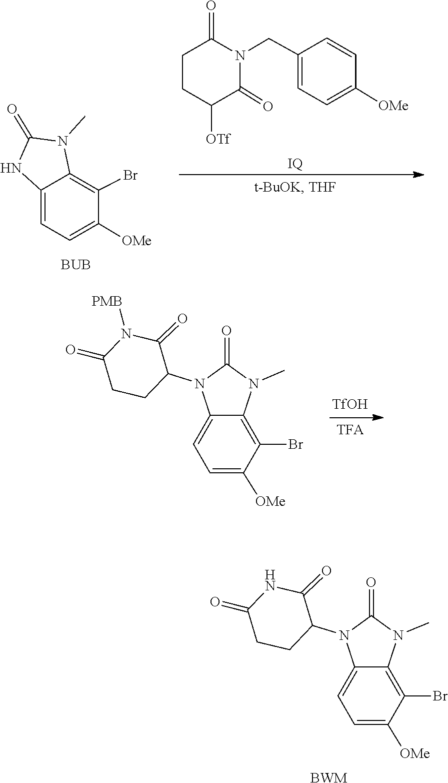

### Step 1—3-(4-Bromo-5-methoxy-3-methyl-2-oxo-benzimidazol-1-yl)-1-[(4-methoxyphenyl) methyl] piperidine-2,6-dione

A solution of 4-bromo-5-methoxy-3-methyl-1H-benzimidazol-2-one (518 mg, 2.02 mmol, Intermediate BUB) in THF (5 mL) was stirred at −10° C., then the t-BuOK (407 mg, 3.63 mmol) was added and the mixture was stirred at −10° C. for 1 hr. Then the solution of [1-[(4-methoxyphenyl) methyl]-2, 6-dioxo-3-piperidyl]trifluoromethanesulfonate (1.00 g, 2.62 mmol, Intermediate IQ) in THF (4 mL) was added dropwise into the above solution. The mixture was stirred at −10° C. for 5 hrs. On completion, the mixture was quenched by NH₄Cl (10 mL), diluted with H₂O (40 mL), then extracted with EA (2×50 mL). Then the mixture was washed with brine (2×50 mL), dried with anhydrous Na₂SO₄ and concentrated in vacuo. The crude product was purified by reversed phase (0.1% FA) to give the title compound (740 mg, 75% yield) as green solid. ¹H NMR (400 MHz, DMSO-d₆) δ 7.20 (d, J=8.8 Hz, 2H), 7.00 (d, J=8.4 Hz, 1H), 6.87-6.84 (m, 2H), 6.77 (d, J=8.8 Hz, 1H), 5.52 (dd, J=5.6, 13.2 Hz, 1H), 4.85-4.72 (m, 2H), 3.82 (s, 3H), 3.72 (s, 3H), 3.64 (s, 3H), 3.10-2.98 (m, 1H), 2.85-2.77 (m, 1H), 2.77-2.69 (m, 1H), 2.10-2.01 (m, 1H). LC-MS (ESI⁺) m/z 489.9 (M+H)⁺.

### Step 2—3-(4-Bromo-5-methoxy-3-methyl-2-oxo-benzimidazol-1-yl) piperidine-2, 6-dione

To a solution of 3-(4-bromo-5-methoxy-3-methyl-2-oxo-benzimidazol-1-yl)-1-[(4-methoxyphenyl) methyl]piperidine-2, 6-dione (800 mg, 1.64 mmol) in mixture solvent of TfOH (200 uL) and TFA (1.6 mL). The mixture was then stirred at 70° C. for 2 hrs. On completion, the mixture was concentrated in vacuo, then dissolved in ACN (3 mL) and adjusted mixture pH=5-6 with TEA. The residue was purified by reversed phase (0.1% FA condition) to give the title compound (218 mg, 36% yield) as white solid. ¹H NMR (400 MHz, DMSO-d₆) δ 11.11 (s, 1H), 7.10 (d, J=2.8 Hz, 1H), 6.82 (d, J=8.4 Hz, 1H), 5.37 (dd, J=5.2, 12.4 Hz, 1H), 3.82 (s, 3H), 3.64 (s, 3H), 2.90-2.83 (m, 1H), 2.73-2.62 (m, 2H), 2.05-1.99 (m, 1H). LC-MS (ESI⁺) m/z 370.2 (M+H)⁺.

### Tert-butyl 4-prop-2-ynoxypiperidine-1-carboxylate (Intermediate BWO)

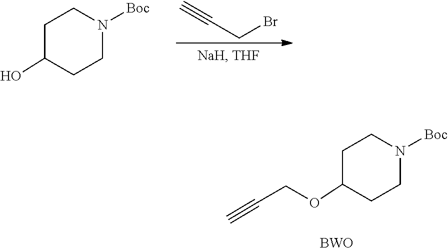

To a solution of tert-butyl 4-hydroxypiperidine-1-carboxylate (2.00 g, 9.94 mmol, CAS #109384-19-2) in anhydrous THF (10 mL) was cooled to 0° C., and subsequently NaH (477 mg, 11.9 mmol, 60% dispersion in mineral oil) was added. The reaction mixture was stirred at 0° C. for 0.5 hr. Then, 3-bromoprop-1-yne (1.18 g, 9.94 mmol, 856 uL) was added. The resulting reaction mixture was stirred at 25° C. for 12 hrs. On completed, the reaction mixture was quenched with water (1 mL), then diluted with ethyl acetate (100 mL). The organic layers were washed with brine (20 mL), dried over anhydrous sodium sulfate, filtered and the filtrate was concentrated in vacuo. The residue was purified by column chromatography to give the title compound (2.38 g, 100% yield) as yellow oil. ¹H NMR (400 MHz, CDCl₃) δ 4.22 (d, J=2.4 Hz, 2H), 3.84-3.75 (m, 2H), 3.73-3.70 (m, 1H), 3.15-3.09 (m, 2H), 2.43 (t, J=2.4 Hz, 1H), 1.93-1.82 (m, 2H), 1.61-1.50 (m, 2H), 1.47 (s, 9H).

### 3-[3,5-Dimethyl-2-oxo-4-(4-piperidyl)benzimidazol-1-yl]-1-methyl-piperidine-2,6-dione (Intermediate BWQ)

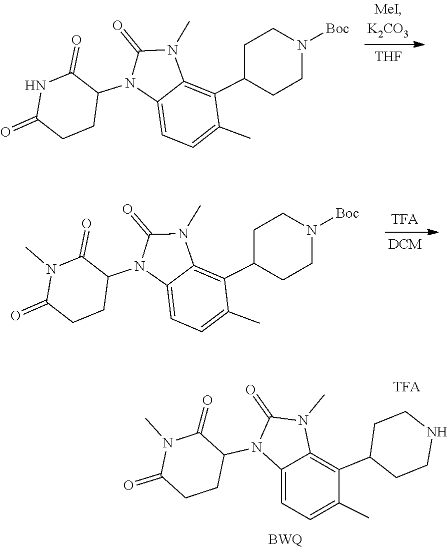

### Step 1—Tert-butyl 4-[3,5-dimethyl-1-(1-methyl-2,6-dioxo-3-piperidyl)-2-oxo-benzimidazol-4-yl]piperidine-1-carboxylate

To a mixture of tert-butyl 4-[1-(2,6-dioxo-3-piperidyl)-3,5-dimethyl-2-oxo-benzimidazol-4-yl]piperidine-1-carboxylate (100 mg, 219 umol, synthesized via Step 1 of Intermediate BQM) in THF (1 mL) was added K₂CO₃ (60.5 mg, 438 umol) and MeI (46.6 mg, 328 umol). The reaction mixture was then stirred at 25° C. for 1 hour. On completion, the residue was diluted with water (20 mL) and extracted with EA (2×20 mL). The combined organic layer was dried over Na₂SO₄, filtered and concentrated in vacuo to give a residue. The residue was purified by reverse phase (0.1% FA condition) to give the title compound (68.0 mg, 65% yield) as white solid. ¹H NMR (400 MHz, DMSO-d₆) δ 6.91-6.86 (m, 1H), 6.84-6.78 (m, 1H), 5.44-5.36 (m, 1H), 4.15-4.00 (m, 2H), 3.62 (s, 3H), 3.57 (s, 1H), 3.05-3.01 (m, 3H), 2.95-2.86 (m, 2H), 2.79-2.67 (m, 2H), 2.39 (s, 2H), 2.28 (s, 1H), 2.13-1.92 (m, 3H), 1.83-1.52 (m, 3H), 1.43 (s, 9H). LC-MS (ESI⁺) m/z 493.2 (M+Na)*.

### Step 2—3-[3,5-Dimethyl-2-oxo-4-(4-piperidyl)benzimidazol-1-yl]-1-methyl-piperidine-2,6-dione

To a mixture of tert-butyl 4-[3,5-dimethyl-1-(1-methyl-2,6-dioxo-3-piperidyl)-2-oxo-benzimidazol-4-yl]piperidine-1-carboxylate (68.0 mg, 144 umol) in DCM (2 mL) was added TFA (16.4 mg, 144 umol). The reaction mixture was then stirred at 25° C. for 1 hour. On completion, the reaction mixture was concentrated in vacuo to give the title compound (70.0 mg, 99% yield, TFA) as brown oil. LC-MS (ESI⁺) m/z 371.1 (M+H)⁺.

### 3-[3-Methyl-4-[(3R)-3-methylpiperazin-1-yl]-2-oxo-benzimidazol-1-yl]piperidine-2,6-dione (Intermediate BWR)

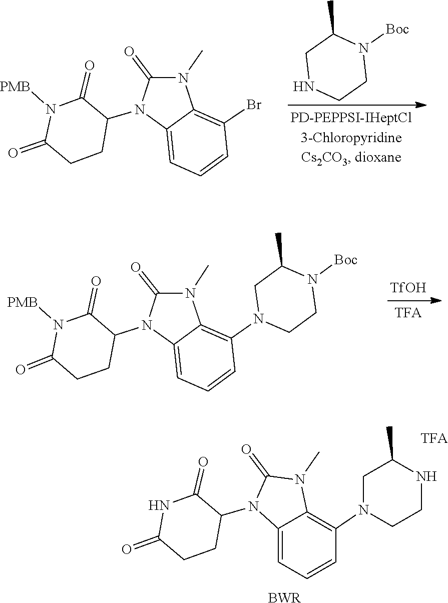

### Step 1—Tert-butyl (2R)-4-[1-[1-[(4-methoxyphenyl)methyl]-2,6-dioxo-3-piperidyl]-3-methyl-2-oxo-benzimidazol-4-yl]-2-methyl-piperazine-1-carboxylate

A mixture of 3-(4-bromo-3-methyl-2-oxo-benzimidazol-1-yl)-1-[(4-methoxyphenyl)methyl]piperidine-2,6-dione (1.00 g, 2.18 mmol, synthesized via Steps 1-4 of Intermediate HP), tert-butyl (2R)-2-methylpiperazine-1-carboxylate (524 mg, 2.62 mmol, CAS #170033-47-3), Pd-PEPPSI-IHeptCl 3-Chloropyridine (212 mg, 218 umol), and Cs₂CO₃ (1.42 g, 4.36 mmol) in dioxane (15 mL) was degassed and purged with N₂ three times. Then the mixture was stirred at 100° C. for 16 hrs under N₂ atmosphere. The residue was purified by column chromatography (SiO₂, PE/EA=10:1 to 2:1) to give the title compound (785 mg, 65% yield) as yellow solid. ¹H NMR (400 MHz, CDCl₃) δ 7.39-7.33 (m, 2H), 6.90-6.85 (m, 2H), 6.84-6.79 (m, 2H), 6.31 (s, 1H), 5.22 (dd, J=5.2, 12.8 Hz, 1H), 5.00-4.91 (m, 2H), 4.55-4.27 (m, 1H), 4.04-3.84 (m, 1H), 3.79 (d, J=5.2 Hz, 6H), 3.41-3.23 (m, 1H), 3.17-2.95 (m, 3H), 2.95-2.75 (m, 2H), 2.74-2.54 (m, 2H), 2.22-2.10 (m, 1H), 1.50 (s, 9H), 1.34 (d, J=6.8 Hz, 3H).

### Step 2—3-[3-Methyl-4-[(3R)-3-methylpiperazin-1-yl]-2-oxo-benzimidazol-1-yl]piperidine-2,6-dione

To a solution of tert-butyl (2R)-4-[1-[1-[(4-methoxyphenyl)methyl]-2,6-dioxo-3-piperidyl]-3-methyl-2-oxo-benzimidazol-4-yl]-2-methyl-piperazine-1-carboxylate (150 mg, 259 umol) in TFA (3 mL) was added TfOH (0.6 mL). The mixture was then stirred at 70° C. for 3 hrs. On completion, the mixture was concentrated in vacuo to give the title compound (110 mg, 90% yield) as black brown oil. LC-MS (ESI+) m/z 358.3 (M+H)⁺.

### 3-[4-(3,8-Diazabicyclo[3.2.1]octan-3-yl)-3-methyl-2-oxo-benzimidazol-1-yl]piperidine-2,6-dione (Intermediate BWS)

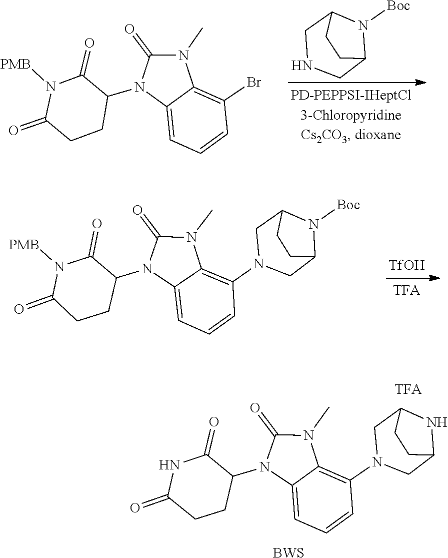

### Step 1—Tert-butyl 3-[1-[1-[(4-methoxyphenyl)methyl]-2,6-dioxo-3-piperidyl]-3-methyl-2-oxo-benzimidazol-4-yl]-3,8-diazabicyclo[3.2.1]octane-8-carboxylate

To a solution of 3-(4-bromo-3-methyl-2-oxo-benzimidazol-1-yl)-1-[(4-methoxyphenyl)methyl]piperidine-2,6-dione (1.00 g, 2.18 mmol, synthesized via Steps 1-4 of Intermediate HP) and tert-butyl 3,8-diazabicyclo[3.2.1]octane-8-carboxylate (462 mg, 2.18 mmol, CAS #149771-44-8) in dioxane (10 mL) was added PD-PEPPSI-IHeptCl 3-Chloropyridine (200 mg, 10.9 umol) and Cs₂CO₃ (1.42 g, 4.36 mmol). Then the reaction mixture was stirred at 100° C. for 16 hrs under N₂. On completion, the reaction mixture was filtered and the filtrate was concentrated in vacuo to give a residue. The residue was purified by reverse phase (0.1% FA condition) to give the title compound (700 mg, 54% yield) as a white solid. ¹H NMR (400 MHz, DMSO-d₆) δ 7.21 (d, J=8.4 Hz, 2H), 7.03-6.92 (m, 2H), 6.90-6.80 (m, 3H), 5.51 (dd, J=5.6, 13.2 Hz, 1H), 4.90-4.70 (m, 2H), 4.19 (s, 2H), 3.72 (s, 3H), 3.71 (s, 3H), 3.11-3.01 (m, 1H), 3.00-2.86 (m, 4H), 2.85-2.77 (m, 1H), 2.76-2.65 (m, 1H), 2.06-1.96 (m, 3H), 1.93-1.83 (m, 2H), 1.45 (s, 9H); LCMS (ESI⁺) m/z 590.4 (M+H)⁺.

### Step 2—3-[4-(3,8-Diazabicyclo[3.2.1]octan-3-yl)-3-methyl-2-oxo-benzimidazol-1-yl]piperidine-2,6-dione

To a mixture of tert-butyl 3-[1-[1-[(4-methoxyphenyl)methyl]-2,6-dioxo-3-piperidyl]-3-methyl-2-oxo-benzimidazol-4-yl]-3,8-diazabicyclo[3.2.1]octane-8-carboxylate (153 mg, 260 umol) in TFA (1.5 mL) was added TfOH (510 mg, 3.40 mmol, 300 uL). Then the reaction mixture was stirred at 70° C. for 3 hrs. On completion, the reaction mixture was concentrated in vacuo to give title compound (120 mg, 95% yield, TFA) as brown oil. LCMS (ESI⁺) m/z 370.2 (M+H)⁺.

### 3-[4-[(1S, 4S)-2, 5-Diazabicyclo[2.2.1]heptan-2-yl]-3-methyl-2-oxo-benzimidazol-1-yl]piperidine-2, 6-dione (Intermediate BWT)

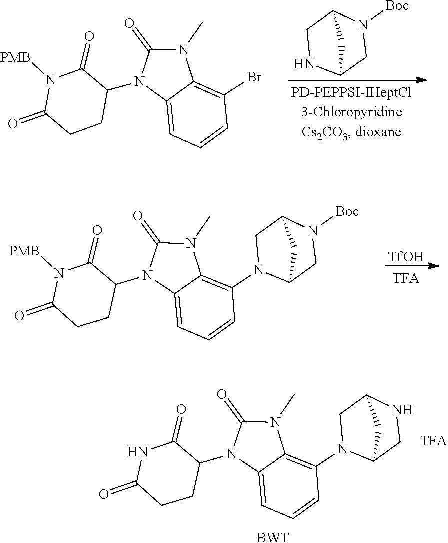

### Step 1—Tert-butyl (1S, 4S)-5-[1-[1-[(4-methoxyphenyl)methyl]-2,6-dioxo-3-piperidyl]-3-methyl-2-oxo-benzimidazol-4-yl]-2,5-diazabicyclo[2.2.1]heptane-2-carboxylate

To a solution of 3-(4-bromo-3-methyl-2-oxo-benzimidazol-1-yl)-1-[(4-methoxyphenyl)methyl]piperidine-2,6-dione (1.00 g, 2.18 mmol, synthesized via Steps 1-4 of Intermediate HP) in dioxane (15 mL) was added PD-PEPPSI-IHeptCl 3-Chloropyridine (212 mg, 218 umol), Cs₂CO₃ (1.42 g, 4.36 mmol) and tert-butyl (1S,4S)-2,5-diazabicyclo[2.2.1]heptane-2-carboxylate (519 mg, 2.62 mmol, CAS #113451-59-5). The mixture was degassed and purged with N₂ three times, and then the mixture was stirred at 100° C. for 8 hrs under N₂ atmosphere. On completion, the mixture was filtered and the cake was washed with ethyl acetate. Then the mixture was concentrated to give a residue, and the residue was purified by column chromatography (SiO₂, Petroleum ether/Ethyl acetate=2:1 to 1:2). The combined organic layers were concentrated under reduced pressure to give the title compound (581 mg, 46% yield) as light pink solid. ¹H NMR (400 MHz, CDCl₃) δ 7.30 (dd, J=1.6, 8.4 Hz, 2H), 6.83-6.72 (m, 4H), 6.20 (d, J=6.8 Hz, 1H), 5.19-5.08 (m, 1H), 4.95-4.84 (m, 2H), 4.56-4.37 (m, 1H), 3.76 (s, 1H), 3.73 (d, J=2.0 Hz, 3H), 3.66 (s, 3H), 3.56-3.46 (m, 1H), 3.45-3.35 (m, 1H), 3.23-3.07 (m, 2H), 2.97-2.89 (m, 1H), 2.82-2.69 (m, 1H), 2.62-2.47 (m, 1H), 2.13-2.00 (m, 2H), 1.92-1.84 (m, 1H), 1.45 (d, J=9.6 Hz, 9H); LC-MS (ESI⁺) m/z 576.3 (M+H)⁺.

### Step 2—3-[4-[(1S, 4S)-2, 5-Diazabicyclo[2.2.1]heptan-2-yl]-3-methyl-2-oxo-benzimidazol-1-yl]piperidine-2, 6-dione

To a solution of tert-butyl (1S, 4S)-5-[1-[1-[(4-methoxyphenyl)methyl]-2,6-dioxo-3-piperidyl]-3-methyl-2-oxo-benzimidazol-4-yl]-2,5-diazabicyclo[2.2.1]heptane-2-carboxylate (280 mg, 486 umol) in TFA (4 mL) was added TfOH (109 mg, 729 umol). Then the mixture was stirred at 70° C. for 8 hrs. On completion, the mixture was concentrated to give the title compound (164 mg, 94% yield, TFA salt) as black oil. LC-MS (ESI⁺) m/z 356.4 (M+H)⁺.

### Step 3—Tert-butyl (1S, 4S)-5-[1-(2,6-dioxo-3-piperidyl)-3-methyl-2-oxo-benzimidazol-4-yl]-2,5-diazabicyclo[2.2.1]heptane-2-carboxylate

To a solution of 3-[4-[(1S, 4S)-2, 5-diazabicyclo[2.2.1]heptan-2-yl]-3-methyl-2-oxo-benzimidazol-1-yl]piperidine-2,6-dione (160 mg, 450 umol) in ACN (5 mL) was added Boc₂O (147 mg, 675 umol) and TEA (45.5 mg, 450 umol). Then the mixture was stirred at 25° C. for 3 hrs. On completion, the mixture was diluted with H₂O (8 mL) and extracted with ethyl acetate (8 mL×2). The combined organic layers were filtered and concentrated under reduced pressure to give a residue to give the title compound (190 mg, 92% yield) as brown solid. LC-MS (ESI⁺) m/z 456.0 (M+H)⁺.

### Step 4—3-[4-[(1S, 4S)-2, 5-Diazabicyclo[2.2.1]heptan-2-yl]-3-methyl-2-oxo-benzimidazol-1-yl]piperidine-2, 6-dione

To a solution of tert-butyl (1S, 4S)-5-[1-(2, 6-dioxo-3-piperidyl)-3-methyl-2-oxo-benzimidazol-4-yl]-2, 5-diazabicyclo[2.2.1]heptane-2-carboxylate (190 mg, 417 umol) in DCM (3 mL) was added TFA (770 mg, 6.75 mmol). The mixture was stirred at 25° C. for 0.5 hr. On completion, the mixture was concentrated to give the title compound (140 mg, 94% yield, TFA) as black oil. LC-MS (ESI⁺) m/z 356.2 (M+H)⁺.

### N-[2-(4-formylcyclohexyl)thiazolo[4,5-c]pyridin-6-yl]-6-(trifluoromethyl)pyridine-2-carboxamide (Intermediate BWU)

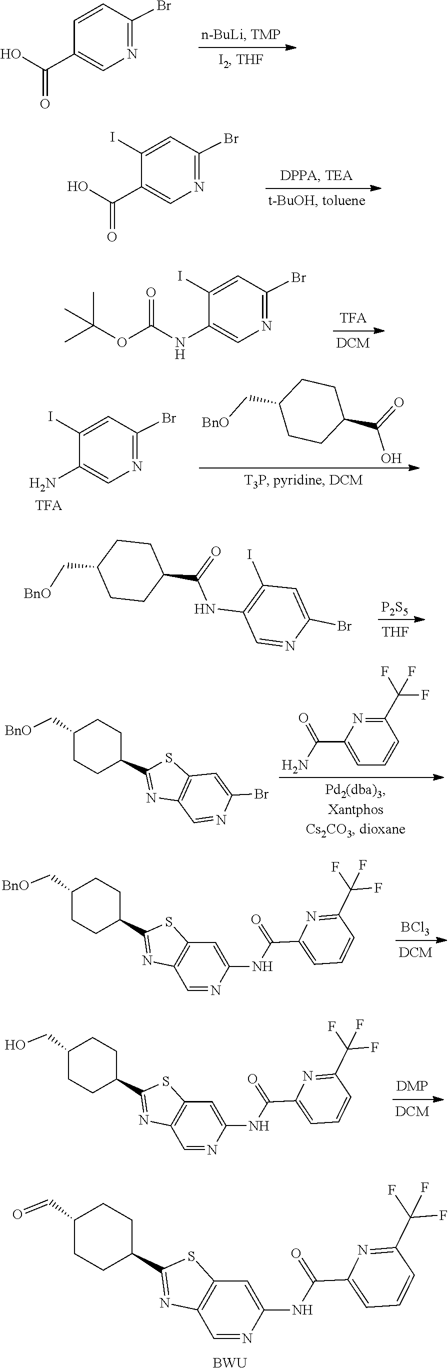

### Step 1—6-Bromo-4-iodo-pyridine-3-carboxylic acid

A dry three neck-round bottom flask (250 mL) containing TMP (10.4 g, 74.2 mmol) and THF (50 mL) under N₂ was cooled to below −55° C. Then, n-BuLi (2.5 M, 29.7 mL) was added dropwise to the cooled solution. The suspension was stirred for 30 mins at below −40° C. Then 6-bromopyridine-3-carboxylic acid (5 g, 24.7 mmol, CAS #6311-35-9) was added quickly in four portions and the suspension was stirred at −20° C. for 2 hrs, over which time the reaction turned from orange to brown. The reaction mixture was then cooled to below −50° C. and quickly transferred into a pre-cooled solution of I₂ (18.8 g, 74.2 mmol) in THF (50 mL) under N₂. The resulting mixture was allowed to warm to 25° C. and stirred for 12 hrs. On completion, the reaction mixture was quenched with NH₄Cl (100 mL), then extracted with DCM (2×100 mL). The aqueous phase was separated and the pH adjust to 2 by the addition of 1N HCl, then extracted with EA (2×100 mL). The organic phase was dried over Na₂SO₄, filtered and the filtrate was concentrated in vacuo to give the title compound (3.2 g, 39% yield) as yellow solid. ¹H NMR (400 MHz, DMSO-d₆) δ 14.13-13.56 (m, 1H), 8.61 (s, 1H), 8.35 (s, 1H).

### Step 2—Tert-butyl N-(6-bromo-4-iodo-3-pyridyl)carbamate

To a solution of 6-bromo-4-iodo-pyridine-3-carboxylic acid (3 g, 9.15 mmol) and TEA (2.78 g, 27.4 mmol, 3.82 mL) in t-BuOH (15 mL) and toluene (15 mL) was added DPPA (3.78 g, 13.7 mmol, 2.97 mL). Then the reaction mixture was stirred at 110° C. for 3 hrs. On completion, the reaction mixture was diluted with H₂O (30 mL), then extracted with EA (2×50 mL). The organic phase was combined dried with Na₂SO₄, and then filtered and the filtrate was concentrated in vacuo to give a residue. The residue was purified by reverse phase (0.10% FA condition) to give the title compound (2.75 g, 75% yield) as yellow solid. ¹H NMR (400 MHz, CDCl₃) δ 8.95 (s, 1H), 7.87 (s, 1H), 6.65 (s, 1H), 1.55 (s, 9H); LC-MS (ESI⁺) m/z 398.8 (M+H)⁺.

### Step 3—6-Bromo-4-iodo-pyridin-3-amine

To a solution of tert-butyl N-(6-bromo-4-iodo-3-pyridyl)carbamate (2.75 g, 6.89 mmol) in DCM (30 mL) was added TFA (9.63 g, 84.4 mmol, 6.25 mL). Then the reaction mixture was stirred at 25° C. for 30 mins. On completion, the reaction mixture was concentrated in vacuo to give a residue. The residue was diluted with a saturated solution of NaHCO₃ to adjust pH=9, then extracted with EA (2×50 mL). The combined organic layer was dried over Na₂SO₄, filtered and the filtrate was concentrated in vacuo to give the title compound (2 g, 97% yield) as yellow solid. ¹H NMR (400 MHz, CDCl₃) δ 7.80 (s, 1H), 7.73 (s, 1H), 4.50-3.68 (s, 2H); LC-MS (ESI⁺) m/z 298.8 (M+H)⁺.

### Step 4—4-(Benzyloxymethyl)-N-(6-bromo-4-iodo-3-pyridyl)cyclohexanecarboxamide

To a solution of 4-(benzyloxymethyl)cyclohexanecarboxylic acid (1.20 g, 4.83 mmol, synthesized via Steps 1-3 of Intermediate BAU) and 6-bromo-4-iodo-pyridin-3-amine (1.37 g, 4.58 mmol) in DCM (20 mL) was added pyridine (1.11 mL, 13.7 mmol) and T₃P (5.82 g, 9.15 mmol). Then the reaction mixture was stirred at 25° C. for 12 hrs. On completion, the reaction mixture was diluted with H₂O (30 mL) and extracted with DCM mL (2×40 mL). The combined organic layers were washed with brine (20 mL), dried over Na₂SO₄, filtered and concentrated in vacuo to give the title compound (2.3 g, 94% yield) as white solid. LC-MS (ESI⁺) m/z 530.8 (M+H)⁺.

### Step 5—2-[4-(Benzyloxymethyl)cyclohexyl]-6-bromo-thiazolo[4,5-c]pyridine

To a solution of 4-(benzyloxymethyl)-N-(6-bromo-4-iodo-3-pyridyl)cyclohexanecarboxamide (1.7 g, 3.21 mmol) in THF (20 mL) was added P₂S₅(785 mg, 3.53 mmol, 375 uL), then the reaction mixture was stirred at 25° C. for 16 hrs. On completion, the reaction mixture was diluted with H₂O (50 mL), then extracted with DCM (2×80 mL). The organic phase was washed with brine (50 mL), then dried over Na₂SO₄, filtered and the filtrate was concentrated in vacuo to give a residue. The residue was purified by reverse phase (0.1% FA condition) to give the title compound (800 mg, 59% yield) as light yellow solid. ¹H NMR (400 MHz, DMSO-d₆) δ 9.00 (s, 1H), 8.45 (s, 1H), 7.41-7.21 (m, 5H), 4.46 (s, 2H), 3.30 (d, J=6.4 Hz, 2H), 3.19-3.06 (m, 1H), 2.18 (d, J=11.6 Hz, 2H), 1.89 (d, J=10.8 Hz, 2H), 1.72-1.65 (m, 1H), 1.64-1.53 (m, 2H), 1.22-1.10 (m, 2H); LC-MS (ESI⁺) m/z 417.4 (M+H)⁺.

### Step 6—N-[2-[4-(benzyloxymethyl)cyclohexyl]thiazolo[4,5-c]pyridin-6-yl]-6-(trifluoromethyl) pyridine-2-carboxamide

To a solution of 2-[4-(benzyloxymethyl)cyclohexyl]-6-bromo-thiazolo[4,5-c]pyridine (750 mg, 1.80 mmol), 6-(trifluoromethyl)pyridine-2-carboxamide (375 mg, 1.98 mmol, CAS #22245-84-7) in dioxane (10 mL) was added Cs₂CO₃ (1.17 g, 3.59 mmol), Pd₂(dba)₃ (164 mg, 179 umol) and Xantphos (103 mg, 179 umol). Then the reaction mixture was stirred at 80° C. for 12 hrs under N₂. On completion, the mixture was filtered and the filtrate was concentrated in vacuo to give a residue. The residue was purified by reverse phase (0.1% FA condition) to give the title compound (460 mg, 48% yield) as yellow solid. ¹H NMR (400 MHz, DMSO-d₆) δ 10.39 (s, 1H), 9.01 (s, 1H), 8.92 (s, 1H), 8.51-8.44 (m, 1H), 8.44-8.37 (m, 1H), 8.24 (d, J=7.6 Hz, 1H), 7.40-7.27 (m, 5H), 4.51-4.41 (m, 2H), 3.30 (d, J=6.4 Hz, 2H), 3.16-3.04 (m, 1H), 2.19 (d, J=10.8 Hz, 2H), 1.90 (d, J=10.8 Hz, 2H), 1.73-1.54 (m, 3H), 1.23-1.11 (m, 2H).

### Step 7—N-[2-[4-(hydroxymethyl)cyclohexyl]thiazolo[4,5-c]pyridin-6-yl]-6-(trifluoromethyl)pyridine-2-carboxamide

To a solution of N-[2-[4-(benzyloxymethyl)cyclohexyl]thiazolo[4,5-c]pyridin-6-yl]-6-(trifluoromethyl) pyridine-2-carboxamide (400 mg, 759 umol) in DCM (10 mL) was added BCl₃ (1 M, 2.28 mL) dropwise at 0° C., then the reaction mixture was stirred at 0° C. for 1 hr. On completion, the reaction mixture was diluted with H₂O (20 mL) and extracted with DCM mL (2×30 mL). The combined organic layers were washed with brine (20 mL), dried over Na₂SO₄, filtered and concentrated in vacuo to give a residue. The residue was purified by reverse phase (0.1% FA condition) to give the title compound (280 mg, 84% yield) as a white solid. ¹H NMR (400 MHz, DMSO-d₆) δ 10.39 (s, 1H), 9.01 (d, J=0.4 Hz, 1H), 8.92 (d, J=0.8 Hz, 1H), 8.51-8.45 (m, 1H), 8.45-8.38 (m, 1H), 8.24 (dd, J=0.8, 7.6 Hz, 1H), 4.46 (t, J=5.2 Hz, 1H), 3.27 (t, J=5.6 Hz, 2H), 3.15-3.03 (m, 1H), 2.26-2.13 (m, 2H), 1.94-1.82 (m, 2H), 1.65-1.52 (m, 2H), 1.50-1.36 (m, 1H), 1.16-1.04 (m, 2H).

### Step 8—N-[2-(4-formylcyclohexyl)thiazolo[4,5-c]pyridin-6-yl]-6-(trifluoromethyl)pyridine-2-carboxamide

To a solution of N-[2-[4-(hydroxymethyl)cyclohexyl]thiazolo[4,5-c]pyridin-6-yl]-6-(trifluoromethyl) pyridine-2-carboxamide (55.0 mg, 126 umol) in DCM (1 mL) was added DMP (64.1 mg, 151 umol), then the reaction mixture was stirred at 25° C. for 1 hr. On completion, the reaction mixture was quenched with Na₂S₂O₃ (15 mL) and extracted with DCM (2×30 mL). The combined organic phase was washed with NaHCO₃ and brine (2×15 mL), dried with anhydrous Na₂SO₄, filtered and concentrated in vacuo to give the title compound (50.0 mg, 91% yield) as yellow solid. LC-MS (ESI⁺) m/z 435.0 (M+H)⁺.

### Tert-butyl 4-(4, 4, 5, 5-tetramethyl-1, 3, 2-dioxaborolan-2-yl)-2-(trifluoromethyl)-3, 6-dihydro-2H-pyridine-1-carboxylate (Intermediate BWV)

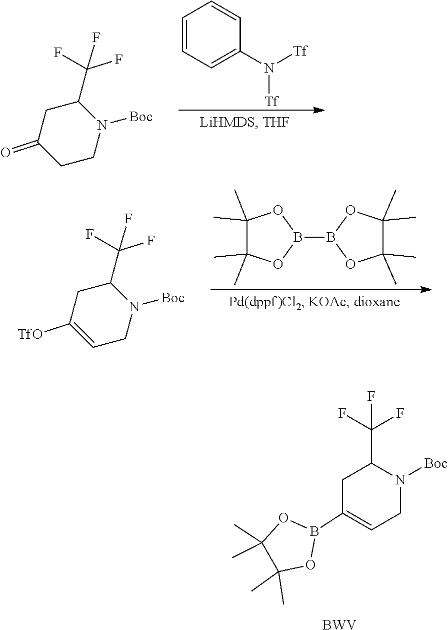

### Step 1—Tert-butyl 2-(trifluoromethyl)-4-(trifluoromethylsulfonyloxy)-3, 6-dihydro-2H-pyridine-1-carboxylate

To a solution of tert-butyl 4-oxo-2-(trifluoromethyl)piperidine-1-carboxylate (3.50 g, 13.1 mmol, CAS #1245648-32-1) in THF (30 mL), LiHMDS (1 M, 19.6 mL) was dropwise added into the above solution at −70° C. and stirred for 2 hrs. Then the solution of 1,1,1-trifluoro-N-phenyl-N-(trifluoromethylsulfonyl) methanesulfonamide (5.61 g, 15.7 mmol, CAS #37595-74-7) in THF (50 mL) was dropwise added into above solution at −70° C., then the mixture was stirred at −70-25° C. for 16 hrs. On completion, the mixture was quenched with saturation solution of NH₄Cl (100 mL), then extracted with EA (3×100 mL), washed with brine (3×100 mL), dried with anhydrous Na₂SO₄, filtered and concentrated in vacuo. The residue was purified by column chromatography to give the title compound (3.10 g, 59% yield) as colorless oil. ¹H NMR (400 MHz, CDCl₃) δ 5.83 (s, 1H), 5.43-5.07 (m, 1H), 4.56-4.21 (m, 1H), 3.35-3.07 (m, 1H), 2.60 (s, 1H), 2.38-2.23 (m, 1H), 1.50 (s, 9H).

### Step 2—Tert-butyl 4-(4, 4, 5, 5-tetramethyl-1, 3, 2-dioxaborolan-2-yl)-2-(trifluoromethyl)-3, 6-dihydro-2H-pyridine-1-carboxylate

To a solution of tert-butyl 2-(trifluoromethyl)-4-(trifluoromethylsulfonyloxy)-3,6-dihydro-2H-pyridine-1-carboxylate (1.25 g, 3.13 mmol) and 4, 4, 5, 5-tetramethyl-2-(4, 4, 5, 5-tetramethyl-1, 3, 2-dioxaborolan-2-yl)-1, 3, 2-dioxaborolane (794 mg, 3.13 mmol, CAS #73183-34-3) in dioxane (15 mL) was added Pd(dppf)Cl₂ (229 mg, 313 umol) and KOAc (921 mg, 9.39 mmol). The mixture was degassed and purged with N₂ three times and stirred at N₂ atmosphere at 65° C. for 40 hrs. On completion, the mixture was filtered and concentrated in vauco. The residue was purified by column chromatography to give the title compound (0.90 g, 76% yield) as colorless oil. ¹H NMR (400 MHz, DMSO-d₆) δ 6.31 (s, 1H), 5.25-5.00 (m, 1H), 4.20-3.96 (m, 1H), 2.96-2.64 (m, 1H), 2.19-2.03 (m, 2H), 1.41 (s, 9H), 1.22 (s, 12H).

### 3-[3-Methyl-2-oxo-4-[2-(trifluoromethyl)-4-piperidyl]benzimidazol-1-yl]piperidine-2,6-dione (Intermediate BWW)

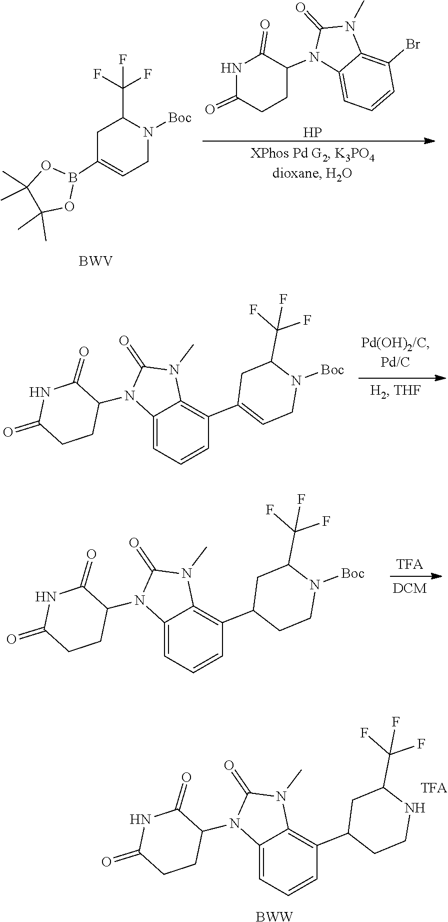

### Step 1—Tert-butyl 4-[1-(2, 6-dioxo-3-piperidyl)-3-methyl-2-oxo-benzimidazol-4-yl]-2-(trifluoromethyl)-3, 6-dihydro-2H-pyridine-1-carboxylate

To a solution of 3-(4-bromo-3-methyl-2-oxo-benzimidazol-1-yl)piperidine-2,6-dione (1.11 g, 3.28 mmol, Intermediate HP) and tert-butyl 4-(4, 4, 5, 5-tetramethyl-1, 3, 2-dioxaborolan-2-yl)-2-(trifluoromethyl)-3, 6-dihydro-2H-pyridine-1-carboxylate (1.61 g, 4.27 mmol, Intermediate BWV) in the mixture solvent of dioxane (20 mL) and H₂O (200 uL) was added XPHOS-PD-G₂ (258 mg, 328 umol), and K₃PO₄ (2.09 g, 9.85 mmol). The mixture was degassed and purged with N₂ three times, and then stirred at for under N₂ atmosphere at 80° C. for 24 hr. On completion, the mixture was filtered and concentrated in vacuo. The crude product was purified by prep-HPLC (reverse phase: 0.10% FA) to give the title compound (670 mg, 40% yield) as brown solid. ¹H NMR (400 MHz, DMSO-d₆) δ 11.11 (s, 1H), 7.13-7.08 (m, 1H), 7.04 (t, J=7.6 Hz, 1H), 6.87 (d, J=7.6 Hz, 1H), 5.78 (s, 1H), 5.40 (dd, J=5.2, 12.8 Hz, 1H), 4.37-4.12 (m, 1H), 3.28 (s, 3H), 3.22 (s, 1H), 3.16-3.02 (m, 1H), 2.95-2.84 (m, 1H), 2.78-2.68 (m, 1H), 2.68-2.61 (m, 1H), 2.44-2.37 (m, 1H), 2.37-2.31 (m, 1H), 2.05-1.97 (m, 1H), 1.46 (s, 9H).

### Step 2—Tert-butyl 4-[1-(2,6-dioxo-3-piperidyl)-3-methyl-2-oxo-benzimidazol-4-yl]-2-(trifluoromethyl) piperidine-1-carboxylate

To a solution of tert-butyl 4-[1-(2,6-dioxo-3-piperidyl)-3-methyl-2-oxo-benzimidazol-4-yl]-2-(trifluoromethyl)-3,6-dihydro-2H-pyridine-1-carboxylate (470 mg, 924 umol) in THF (8 mL) was added Pd/C (250 mg, 924 umol, 10 wt %) and Pd(OH)₂/C (250 mg, 356 umol, 20 wt %) under Ar atmosphere. The mixture was then place under H₂ atmosphere (50 psi) and stirred at 50° C. for 48 hrs. On completion, the mixture was filtered and concentrated in vacuo to give the title compound (230 mg, 48% yield) as white solid. ¹H NMR (400 MHz, DMSO-d₆) δ 11.09 (s, 1H), 7.13-7.09 (m, 1H), 7.08-7.04 (m, 1H), 7.02-6.98 (m, 1H), 5.41-5.36 (m, 1H), 4.95-4.83 (m, 1H), 3.95-3.85 (m, 1H), 3.57-3.53 (m, 3H), 3.26-3.18 (m, 1H), 2.87-2.82 (m, 1H), 2.72-2.62 (m, 2H), 2.24-2.17 (m, 1H), 2.16-2.09 (m, 1H), 2.08-1.94 (m, 3H), 1.65-1.55 (m, 1H), 1.45 (s, 9H). LC-MS (ESI⁺) m/z 511.2 (M+H)⁺.

### Step 3—3-[3-Methyl-2-oxo-4-[2-(trifluoromethyl)-4-piperidyl]benzimidazol-1-yl]piperidine-2,6-dione

To a solution of tert-butyl 4-[1-(2, 6-dioxo-3-piperidyl)-3-methyl-2-oxo-benzimidazol-4-yl]-2-(trifluoromethyl) piperidine-1-carboxylate (200 mg, 391 umol) in DCM (4 mL) was added TFA (616 mg, 5.40 mmol). The mixture was then stirred at 25° C. for 2 hrs. On completion, the mixture was concentrated in vacuo, then dissolved with DMF (1 mL), and the solution adjusted pH=6-7 with TEA. The crude product was purified by prep-HPLC (column: 3_Phenomenex Luna C 18 75*30 mm*3 um; mobile phase: [water (10 mM NH₄HCO₃)-ACN]; B %: 15%-45%, 10 min) to give the title compound (75.0 mg, 46% yield) as white solid. ¹H NMR (400 MHz, DMSO-d₆) δ 11.09 (s, 1H), 7.06-7.01 (m, 2H), 7.01-6.97 (m, 1H), 5.37 (dd, J=5.2, 12.4 Hz, 1H), 3.59 (s, 3H), 3.52-3.48 (m, 1H), 3.10 (d, J=12.4 Hz, 1H), 2.97-2.82 (m, 2H), 2.80-2.62 (m, 3H), 2.59-2.52 (m, 1H), 2.03-1.96 (m, 1H), 1.91 (d, J=11.6 Hz, 1H), 1.79-1.73 (m, 1H), 1.67-1.54 (m, 2H). LC-MS (ESI⁺) m/z 411.2 (M+H)⁺.

### 3-[4-chloro-3-methyl-2-oxo-5-(4-piperidyl)benzimidazol-1-yl]piperidine-2,6-dione (Intermediate BWX)

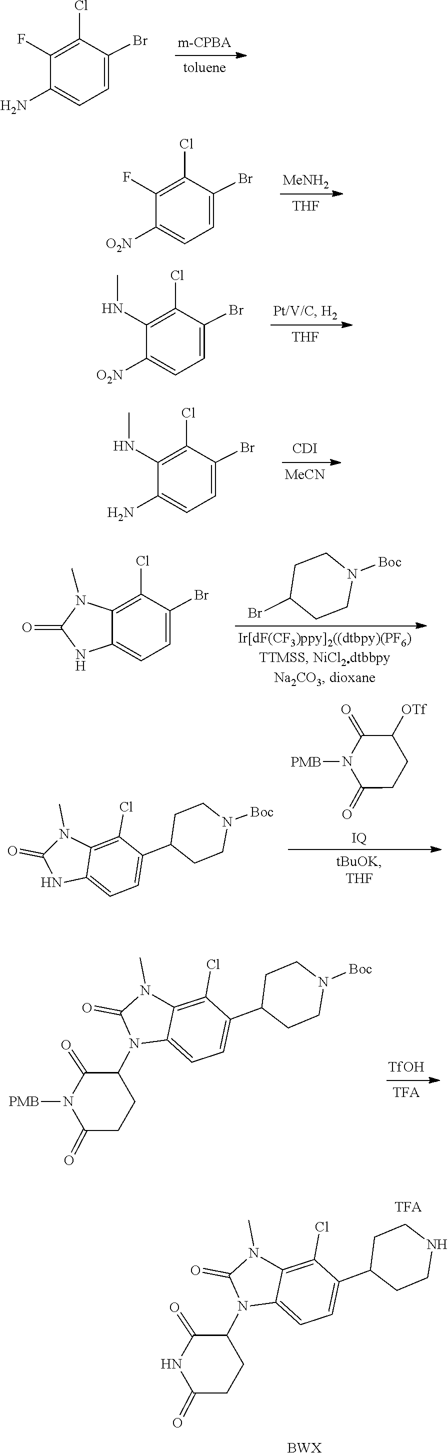

### Step 1—1-bromo-2-chloro-3-fluoro-4-nitro-benzene

To a solution of 4-bromo-3-chloro-2-fluoro-aniline (2 g, 8.91 mmol, CAS #115843-99-7) in toluene (15 mL) was added m-CPBA (7.69 g, 35.6 mmol, 80% solution). The reaction was stirred at 50° C. for 24 hrs. On completion, the reaction was quenched with Na₂SO₃ solution (8 g Na₂SO₃) and stirred at 0° C. for 0.5 hr. The mixture was diluted with EA (250 mL). The organic layer was washed with water (250 mL×3), dried over Na₂SO₄ and filtered. The filtrate was concentrated in vacuo. The residue was purified by column chromatography (SiO₂, Petroleum ether/Ethyl acetate=10/1) to give title compound (0.66 g, 29% yield) as yellow solid. ¹H NMR (400 MHz, CDCl₃) δ=7.81 (dd, J=7.2, 9.2 Hz, 1H), 7.55 (dd, J=2.0, 9.2 Hz, 1H).

### Step 2—3-bromo-2-chloro-N-methyl-6-nitro-aniline

To a solution of 1-bromo-2-chloro-3-fluoro-4-nitro-benzene (1.65 g, 6.48 mmol) in THF (20 mL) was added MeNH₂ (6.48 mL, 2M) in THF at 0° C. The reaction was stirred at 25° C. for 1 hr. On completion, the reaction was diluted with EA (150 mL). The organic layer was washed with water (100 mL×3), dried over Na₂SO₄ and filtered. The filtrate was concentrated in vacuo to give title compound (1.71 g, 99% yield) as red solid. ¹H NMR (400 MHz, CDCl₃) δ 7.69 (d, J=9.2 Hz, 1H), 6.99 (d, J=9.2 Hz, 1H), 6.67 (s, 1H), 3.01 (d, J=2.0 Hz, 3H).

### Step 3—4-bromo-3-chloro-N2-methyl-benzene-1,2-diamine

To a solution of 3-bromo-2-chloro-N-methyl-6-nitro-aniline (1.71 g, 6.44 mmol) in MeOH (20 mL) was added Pt/V/C (1 g, 114 umol, 3 wt %) under N₂. The suspension was degassed under vacuum and purged with H₂ several times. The mixture was stirred under H₂ (15 psi) at 25° C. for 4 hrs. On completion, the reaction was filtered. The filtrate was concentrated in vacuo to give title compound (1.3 g, 85% yield) as yellow oil. ¹H NMR (400 MHz, CDCl₃) δ 7.04 (d, J=8.4 Hz, 1H), 6.44 (d, J=8.4 Hz, 1H), 4.01-3.36 (m, 3H), 2.61 (s, 3H).

### Step 4—5-bromo-4-chloro-3-methyl-1H-benzimidazol-2-one

A mixture of 4-bromo-3-chloro-N2-methyl-benzene-1,2-diamine (1.3 g, 5.52 mmol) and CDI (1.52 g, 9.38 mmol) in MeCN (20 mL) was stirred at 80° C. for 4 hrs. On completion, the mixture was diluted with water (100 mL) and filtered. The filter cake was concentrated in vacuo to give title compound (1.3 g, 90% yield) as yellow solid. ¹H NMR (400 MHz, DMSO-d₆) δ 11.33 (s, 1H), 7.34 (d, J=8.4 Hz, 1H), 6.90 (d, J=8.4 Hz, 1H), 3.56 (s, 3H).

### Step 5—Tert-butyl 4-(4-chloro-3-methyl-2-oxo-1H-benzimidazol-5-yl)piperidine-1-carboxylate

To a 15 mL vial equipped with a stir bar was added 5-bromo-4-chloro-3-methyl-1H-benzimidazol-2-one (1.3 g, 4.97 mmol), tert-butyl 4-bromopiperidine-1-carboxylate (1.71 g, 6.46 mmol, CAS #180695-79-8), Ir[dF(CF₃)ppy]₂(dtbpy)(PF₆) (55.7 mg, 49.7 umol), NiCl₂.dtbbpy (9.89 mg, 24.8 umol), TTMSS (1.24 g, 4.97 mmol) and Na₂CO₃ (1.05 g, 9.94 mmol) in DME (30 mL). The vial was sealed and placed under nitrogen was added. The reaction was stirred and irradiated with a 10 W [455 nm] blue LED lamp (3 cm away), with cooling water to keep the reaction temperature at 25° C. for 14 hrs. On completion, the reaction was diluted with EA (150 mL). The organic layer was washed with water (150 mL×3), dried over Na₂SO₄ and filtered. The filtrate was concentrated in vacuo. The residue was purified by reverse phase. (0.1% FA condition) to give title compound (500 mg, 27% yield) as white solid. ¹H NMR (400 MHz, DMSO-d₆) δ 11.22-10.94 (m, 1H), 7.04-6.82 (m, 2H), 4.14-4.01 (m, 2H), 3.59-3.55 (m, 3H), 3.19-3.11 (m, 1H), 2.92-2.77 (m, 2H), 1.72 (d, J=12.0 Hz, 2H), 1.52-1.44 (m, 2H), 1.41 (s, 9H).

### Step 6—Tert-butyl 4-[4-chloro-1-[1-[(4-methoxyphenyl)methyl]-2,6-dioxo-3-piperidyl]-3-methyl-2-oxo-benzimidazol-5-yl]piperidine-1-carboxylate

To a solution of tert-butyl 4-(4-chloro-3-methyl-2-oxo-1H-benzimidazol-5-yl)piperidine-1-carboxylate (50.0 mg, 136 umol) in THF (0.5 mL) was added t-BuOK (23.0 mg, 205 umol) at 0° C. The reaction was stirred at 0° C. for 0.5 hr. Then, a solution of [1-[(4-methoxyphenyl)methyl]-2,6-dioxo-3-piperidyl]trifluoromethanesulfonate (78.1 mg, 205 umol, Intermediate IQ) in THF (1 mL) was added into the mixture at 0° C. The reaction was stirred at 0° C. for 3.5 hrs. On completion, the reaction was diluted with EA (30 mL). The organic layer was washed with water (30 mL×3), dried over Na₂SO₄ and concentrated in vacuo. The residue was purified by prep-TLC (SiO₂, PE:EA=1:2, P1:R_{f}=0.5) to give title compound (46 mg, 56% yield) as white solid. ¹H NMR (400 MHz, DMSO-d₆) δ 7.20 (d, J=8.8 Hz, 2H), 6.98 (s, 2H), 6.89-6.80 (m, 2H), 5.55 (dd, J=5.6, 13.2 Hz, 1H), 4.85-4.71 (m, 2H), 4.17-4.04 (m, 2H), 3.73 (s, 3H), 3.66 (s, 3H), 3.20-3.14 (m, 1H), 3.12-2.98 (m, 1H), 2.94-2.64 (m, 4H), 2.10-2.00 (m, 1H), 1.73 (d, J=12.4 Hz, 2H), 1.59-1.46 (m, 2H), 1.42 (s, 9H).

### Step 7—3-[4-chloro-3-methyl-2-oxo-5-(4-piperidyl)benzimidazol-1-yl]piperidine-2,6-dione

A mixture of tert-butyl 4-[4-chloro-1-[1-[(4-methoxyphenyl)methyl]-2,6-dioxo-3-piperidyl]-3-methyl-2-oxo-benzimidazol-5-yl]piperidine-1-carboxylate (46 mg, 77.0 umol) and TfOH (340.00 mg, 2.27 mmol) in TFA (1 mL) was stirred at 70° C. for 4 hrs. On completion, the reaction was concentrated in vacuo to give title compound (37.8 mg, 99% yield, TFA) as brown oil. LC-MS (ESI⁺) m/z 377.3 (M+H)⁺.

### 3-[3,4-dimethyl-2-oxo-5-(4-piperidyl)benzimidazol-1-yl]piperidine-2,6-dione (Intermediate BWY)

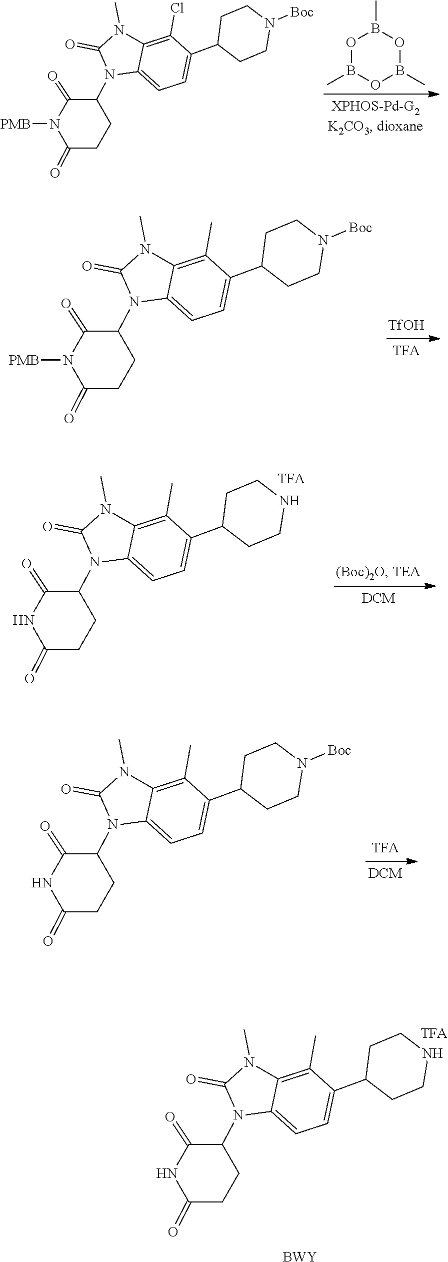

### Step 1—Tert-butyl 4-[1-[1-[(4-methoxyphenyl)methyl]-2,6-dioxo-3-piperidyl]-3,4-dimethyl-2-oxo-benzimidazol-5-yl]piperidine-1-carboxylate

A solution of tert-butyl 4-[4-chloro-1-[1-[(4-methoxyphenyl)methyl]-2,6-dioxo-3-piperidyl]-3-methyl-2-oxo-benzimidazol-5-yl]piperidine-1-carboxylate (200 mg, 334 umol, synthesized via Steps 1-6 of Intermediate BWX), 2,4,6-trimethyl-1,3,5,2,4,6-trioxatriborinane (84.1 mg, 669 umol, CAS #823-96-1), K₂CO₃ (138 mg, 1.00 mmol) and XPHOS-PD-G₂ (26.3 mg, 33.5 umol) in dioxane (3 mL) was stirred at 90° C. for 16 hrs under N₂. On completion, the reaction was filtered and the filtrate was concentrated in vacuo. The residue was purified by reverse phase (0.1% FA condition) to give title compound (190 mg, 98% yield) as pink solid. ¹H NMR (400 MHz, DMSO-d₆) δ 7.20 (d, J=8.4 Hz, 2H), 6.88-6.79 (m, 3H), 6.76 (s, 1H), 5.75 (s, 1H), 5.48 (dd, J=5.2, 12.8 Hz, 1H), 4.85-4.73 (m, 2H), 4.08 (d, J=10.0 Hz, 2H), 3.72 (s, 3H), 3.61 (s, 3H), 3.10-2.93 (m, 3H), 2.75-2.67 (m, 2H), 2.56 (s, 3H), 2.05-1.95 (m, 1H), 1.65 (d, J=12.4 Hz, 2H), 1.51-1.45 (m, 2H), 1.42 (s, 9H).

### Step 2—3-[3,4-dimethyl-2-oxo-5-(4-piperidyl)benzimidazol-1-yl]piperidine-2,6-dione

To a mixture of tert-butyl 4-[1-[1-[(4-methoxyphenyl)methyl]-2,6-dioxo-3-piperidyl]-3,4-dimethyl-2-oxo-benzimidazol-5-yl]piperidine-1-carboxylate (160 mg, 277 umol) in TFA (2 mL) was added TfOH (0.5 mL), then the reaction was stirred at 70° C. for 4 hrs. On completion, the reaction was concentrated in vacuo to give title compound (130 mg, 99% yield, TFA) as brown oil. LC-MS (ESI⁺) m/z 357.4 (M+H)⁺.

### Step 3—Tert-butyl 4-[1-(2,6-dioxo-3-piperidyl)-3,4-dimethyl-2-oxo-benzimidazol-5-yl]piperidine-1-carboxylate

A mixture of 3-[3,4-dimethyl-2-oxo-5-(4-piperidyl)benzimidazol-1-yl]piperidine-2,6-dione (130 mg, 276.34 umol, TFA), (Boc)₂O (90.4 mg, 414 umol) and TEA (279 mg, 2.76 mmol) in DCM (2 mL) was stirred at 25° C. for 2 hrs. On completion, the reaction was concentrated in vacuo. The residue was purified by prep-HPLC (column: Phenomenex luna C18 150*25 mm*10 um; mobile phase: [water(0.225% FA)-ACN]; B %: 36%-66%, 10 min) to give title compound (75 mg, 59% yield) as white solid. ¹H NMR (400 MHz, DMSO-d₆) δ 11.07 (s, 1H), 6.89 (s, 2H), 5.32 (dd, J=5.2, 12.4 Hz, 1H), 4.07 (d, J=10.0 Hz, 2H), 3.61 (s, 3H), 3.30 (s, 1H), 3.02-2.78 (m, 4H), 2.74-2.65 (m, 1H), 2.56 (s, 3H), 2.01-1.92 (m, 1H), 1.66 (d, J=12.0 Hz, 2H), 1.52-1.44 (m, 2H), 1.42 (s, 9H).

### Step 4—3-[3,4-dimethyl-2-oxo-5-(4-piperidyl)benzimidazol-1-yl]piperidine-2,6-dione

To a solution of tert-butyl 4-[1-(2,6-dioxo-3-piperidyl)-3,4-dimethyl-2-oxo-benzimidazol-5-yl]piperidine-1-carboxylate (40 mg, 87.6 umol) in DCM (1 mL) was added TFA (0.3 mL), then the reaction was stirred at 25° C. for 1 hr. On completion, the reaction was concentrated in vacuo to give title compound (41 mg, 99% yield, TFA) as brown oil. LC-MS (ESI⁺) m/z 357.2 (M+H)⁺.

### 2-[4-(Benzyloxymethyl)cyclohexyl]-6-bromo-5-methoxy-1,3-benzothiazole (Intermediate BWZ)

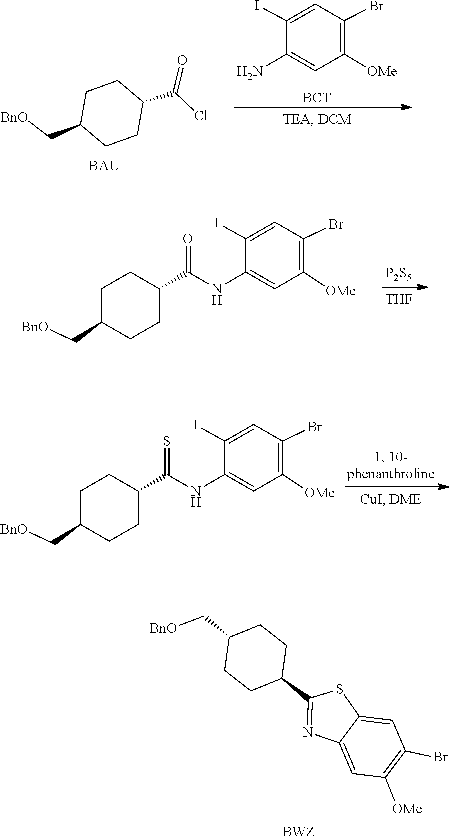

### Step 1—4-(Benzyloxymethyl)-N-(4-bromo-2-iodo-5-methoxy-phenyl)cyclohexanecarboxamide

To a mixture of 4-bromo-2-iodo-5-methoxy-aniline (7.00 g, 21.3 mmol, Intermediate BCT) and TEA (6.48 g, 64.0 mmol) in DCM (70 mL) was added a solution of 4-(benzyloxymethyl)cyclohexanecarbonyl chloride (5.69 g, 21.3 mmol, Intermediate BAU) in DCM (70 mL). The reaction mixture was stirred at 25° C. for 12 hr. On completion, the reaction mixture was diluted with water (30 mL) and extracted with EA (2×80 mL). The combined organic layer was dried over Na₂SO₄, filtered and concentrated in vacuo to give a residue. The residue was purified by column chromatography to give the title compound (11.9 g, 99% yield) as light yellow solid. ¹H NMR (400 MHz, DMSO-d₆) δ 9.26 (s, 1H), 7.99-7.95 (m, 1H), 7.38-7.22 (m, 6H), 4.45 (s, 2H), 3.82-3.79 (m, 3H), 3.27 (d, J=6.0 Hz, 2H), 2.43-2.31 (m, 1H), 1.95 (d, J=12.0 Hz, 2H), 1.88-1.80 (m, 2H), 1.64-1.53 (m, 1H), 1.44 (dq, J=3.6, 12.8 Hz, 2H), 1.09-0.95 (m, 2H). LC-MS (ESI⁺) m/z 558.1 (M+H)⁺.

### Step 2—4-(Benzyloxymethyl)-N-(4-bromo-2-iodo-5-methoxy-phenyl)cyclohexanecarbothioamide

To a mixture of 4-(benzyloxymethyl)-N-(4-bromo-2-iodo-5-methoxy-phenyl)cyclohexanecarboxamide (14.0 g, 25.0 mmol) in THF (140 mL) was added P₂S₅(5.57 g, 25.0 mmol). The reaction mixture was stirred at 25° C. for 2 hrs. On completion, the reaction mixture was filtered and the filtrate was diluted with water (30 mL), then the filtrate was extracted with EA (3×200 mL). The combined organic layer was dried over Na₂SO₄, filtered and concentrated in vacuo to give the title compound (14.4 g, 99% yield) as brown oil. ¹H NMR (400 MHz, DMSO-d₆) δ 11.34-11.28 (m, 1H), 8.03 (s, 1H), 7.38-7.30 (m, 5H), 7.02 (s, 1H), 4.46 (s, 2H), 3.81 (s, 3H), 3.29 (d, J=6.4 Hz, 2H), 2.80-2.74 (m, 1H), 1.93 (s, 2H), 1.86 (d, J=10.8 Hz, 2H), 1.70 (dd, J=2.0, 12.4 Hz, 2H), 1.62-1.58 (m, 1H), 1.13-1.03 (m, 2H).

### Step 6—2-[4-(Benzyloxymethyl)cyclohexyl]-6-bromo-5-methoxy-1,3-benzothiazole

To a mixture of 4-(benzyloxymethyl)-N-(4-bromo-2-iodo-5-methoxy-phenyl)cyclohexanecarbothioamide (14.4 g, 25.0 mmol) in DME (150 mL) was added CuI (477 mg, 2.51 mmol), 1,10-phenanthroline (451 mg, 2.51 mmol). The reaction mixture was then stirred at 40° C. for 12 hrs. On completion, the residue was diluted with water (100 mL), then the residue was extracted with EA (3×200 mL). The combined organic layer was dried over Na₂SO₄, filtered and concentrated in vacuo to give a residue. The residue was purified by column chromatography to give the title compound (11.0 g, 98% yield) as white solid. ¹H NMR (400 MHz, DMSO-d₆) δ 8.30 (s, 1H), 7.64 (s, 1H), 7.38-7.35 (m, 1H), 7.33 (d, J=2.8 Hz, 3H), 7.30-7.27 (m, 1H), 4.46 (s, 2H), 3.91 (s, 3H), 3.29 (d, J=6.0 Hz, 2H), 3.02 (tt, J=3.6, 12.0 Hz, 1H), 2.17-2.12 (m, 2H), 1.88 (dd, J=2.8, 13.6 Hz, 2H), 1.56 (dq, J=3.2, 12.8 Hz, 3H), 1.15 (dd, J=3.6, 12.4 Hz, 2H).

### 3-[4-[1-[[4-(5-amino-6-methoxy-indazol-2-yl) cyclohexyl]methyl]-4-piperidyl]-3-methyl-2-oxo-benzimidazol-1-yl] piperidine-2,6-dione (Intermediate BXA)

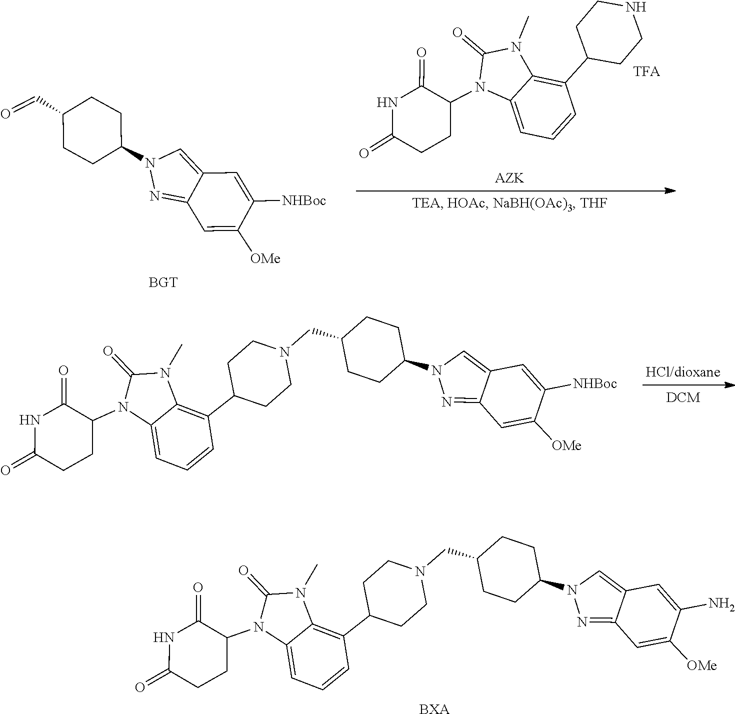

### Step 1—Tert-butyl N-[2-[4-[[4-[1-(2,6-dioxo-3-piperidyl)-3-methyl-2-oxo-benzimidazol-4-yl]-1-piperidyl]methyl]cyclohexyl]-6-methoxy-indazol-5-yl]carbamate

To a solution of 3-[3-methyl-2-oxo-4-(4-piperidyl)benzimidazol-1-yl]piperidine-2,6-dione (1 g, 2.20 mmol, Intermediate AZK) in THF (8 mL) and DMF (1 mL) was added TEA (222 mg, 2.20 mmol) and HOAc (132 mg, 2.20 mmol) until the pH=6. Then tert-butyl (2-((1r, 4r)-4-formylcyclohexyl)-6-methoxy-2H-indazol-5-yl)carbamate (820 mg, 2.20 mmol, Intermediate BGT) was added into the mixture. The reaction was stirred at −10° C. for 30 min, followed by addition of NaBH(OAc)₃ (931 mg, 4.39 mmol). The reaction was then stirred at −10° C. for 2 hrs. On completion, the mixture was quenched with water (0.05 mL) and concentrated in vacuo to give the title compound (820 mg, 53% yield) as a brown solid. ¹H NMR (400 MHz, DMSO-d₆) δ 11.10 (s, 1H), 8.24-8.14 (m, 1H), 7.79 (s, 1H), 7.12-7.00 (m, 2H), 6.99-6.89 (m, 2H), 5.39 (dd, J=5.2, 12.4 Hz, 1H), 4.45-4.33 (m, 1H), 4.03 (m, J=7.1 Hz, 1H), 3.85 (s, 3H), 3.63 (s, 3H), 3.12-2.99 (m, 2H), 2.94-2.84 (m, 1H), 2.77-2.63 (m, 2H), 2.16 (d, J=11.6 Hz, 2H), 2.05-1.94 (m, 8H), 1.94-1.89 (m, 2H), 1.46 (s, 9H), 1.40 (s, 1H), 1.27 (d, J=4.6, 7.3 Hz, 2H), 1.17 (t, J=7.0 Hz, 2H), 0.86-0.81 (m, 2H); LC-MS (ESI⁺) m/z 700.6 (M+H)⁺.

### Step 2—3-[4-[1-[[4-(5-amino-6-methoxy-indazol-2-yl) cyclohexyl]methyl]-4-piperidyl]-3-methyl-2-oxo-benzimidazol-1-yl] piperidine-2,6-dione

To a solution of tert-butyl (2-((1r, 4r)-4-((4-(1-(2,6-dioxopiperidin-3-yl)-3-methyl-2-oxo-2,3-dihydro-1H-benzo[d]imidazol-4-yl)piperidin-1-yl)methyl)cyclohexyl)-6-methoxy-2H-indazol-5-yl)carbamate (100 mg, 143 umol) in DCM (1.5 mL) was added HCl/dioxane (4 M, 3 mL). The mixture was stirred at 10° C. for 2 hrs. On completion, the mixture was concentrated in vacuo to give the title compound (66 mg, 77% yield) as a brown solid. LC-MS (ESI⁺) m/z 600.6 (M+H)⁺.

### 6-(Trifluoromethyl)nicotinic acid (CAS #231291-22-8) (Intermediate BXB)

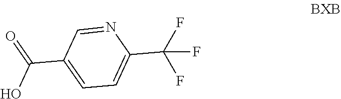 [SMILES: O=C(O)c1ccc(C(F)(F)F)nc1]

### 2-Fluorobenzoic acid (CAS #445-29-4) (Intermediate BXC)

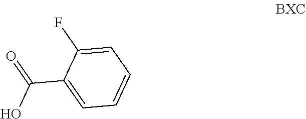

### 4-(trifluoromethyl)thiazole-2-carboxylic acid (CAS #944900-55-4) (Intermediate BXD)

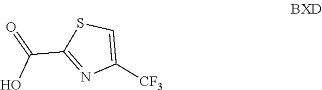 [SMILES: O=C(O)c1nc(C(F)(F)F)cs1]

### N-[2-(4-formylcyclohexyl)-7-methoxy-indazol-5-yl]-6-(trifluoromethyl)pyridine-2-carboxamide (Intermediate BXE)

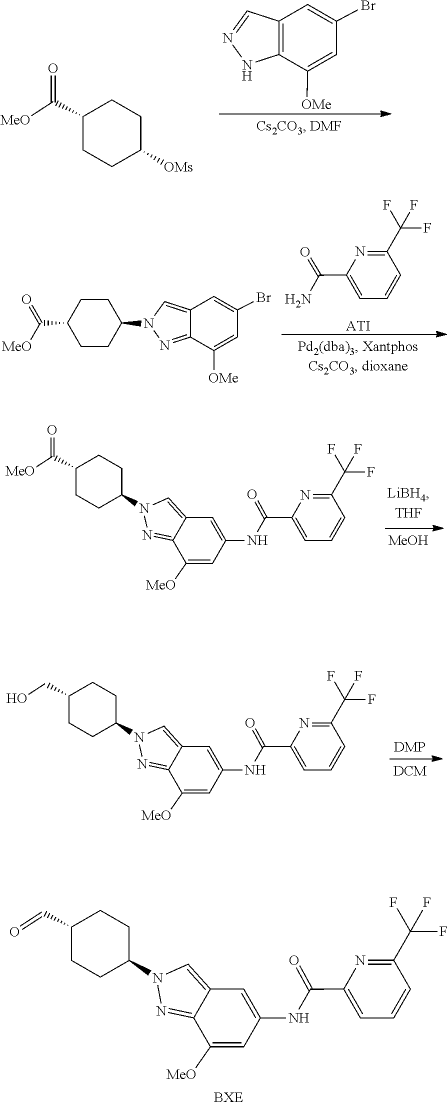

### Step 1—Methyl 4-(5-bromo-7-methoxy-indazol-2-yl)cyclohexanecarboxylate

To a solution of methyl 4-methylsulfonyloxycyclohexanecarboxylate (2.08 g, 1.76 mmol, synthesized via Step 1 of Intermediate QS) and 5-bromo-7-methoxy-2H-indazole (1.00 g, 881 umol, CAS #1374652-62-6) in DMF (2 mL) was added Cs₂CO₃ (574 mg, 1.76 mmol). The mixture was then stirred at 80° C. for 16 hrs. On completion, the reaction mixture was filtered and concentrated under reduced pressure to give a residue. The reaction mixture was purified by reversed phase flash (0.1% FA condition) to give the title compound (198 mg, 12% yield) as a brown solid. ¹H NMR (400 MHz, CHLOROFORM-d) 6=7.85 (s, 1H), 7.38 (d, J=1.2 Hz, 1H), 6.63 (d, J=1.6 Hz, 1H), 4.42 (tt, J=4.0, 12.0 Hz, 1H), 4.01 (s, 3H), 3.71 (s, 3H), 2.44 (tt, J=3.6, 12.4 Hz, 1H), 2.39-2.32 (m, 2H), 2.26-2.18 (m, 2H), 2.07-1.96 (m, 2H), 1.68 (dq, J=3.3, 13.2 Hz, 2H). LC-MS (ESI+) m/z 369.0 (M+H)⁺.

### Step 2—Methyl 4-[7-methoxy-5-[[6-(trifluoromethyl)pyridine-2-carbonyl]amino]indazol-2-yl] cyclohexanecarboxylate

To a solution of methyl 4-(5-bromo-7-methoxy-indazol-2-yl)cyclohexanecarboxylate (332 mg, 880 umol) and 6-(trifluoromethyl)pyridine-2-carboxamide (167 mg, 879.54 umol, Intermediate ATI) in dioxane (10 mL) was added Xantphos (102 mg, 175.91 umol), Cs₂CO₃ (860 mg, 2.64 mmol) and Pd₂(dba)₃ (80.5 mg, 88.0 umol). The mixture was then stirred under N₂ at 110° C. for 16 hrs. On completion, the reaction mixture was filtered and concentrated under reduced pressure to give a residue. The residue was purified by prep-HPLC (column: Phenomenex Luna C18 150*25 mm*10 um; mobile phase: [water(0.05% HCl)-ACN]; B %: 42%-72%, 10 min) to give the title compound (95.0 mg, 17% yield) as an off-white solid. LC-MS (ESI⁺) m/z 447.3 (M+H)⁺.

### Step 3—N-[2-[4-(hydroxymethyl)cyclohexyl]-7-methoxy-indazol-5-yl]-6-(trifluoromethyl)pyridine-2-carboxamide

A mixture of methyl methyl 4-[7-methoxy-5-[[6-(trifluoromethyl)pyridine-2-carbonyl]amino]indazol-2-yl]cyclohexanecarboxylate (90.0 mg, 145 umol) in THF (0.8 mL) and MeOH (0.2 mL) was degassed and purged with N₂ for three times. Then to the mixture was added LiBH₄ (100 mg, 4.59 mmol), and then the mixture was stirred at 60° C. for 16 hrs under N₂ atmosphere. On completion, the reaction mixture was quenched by addition H₂O (2 mL) at 25° C., and then extracted with EA 15 mL (3×5 mL). The combined organic layers were washed with brine (2×5 mL), dried over Na₂SO₄, filtered and concentrated under reduced pressure to give a residue. The residue was purified by prep-TLC (SiO₂, DCM:MeOH=15:1) to give the title compound (44.0 mg, 56% yield) as a yellow solid. LC-MS (ESI+) m/z 449.4 (M+H)⁺.

### Step 4—N-[2-(4-formylcyclohexyl)-7-methoxy-indazol-5-yl]-6-(trifluoromethyl)pyridine-2-carboxamide

To a solution of N-[2-[4-(hydroxymethyl)cyclohexyl]-7-methoxy-indazol-5-yl]-6-(trifluoromethyl) pyridine-2-carboxamide (44.0 mg, 98.1 umol) in DCM (1 mL) was added DMP (49.9 mg, 118 umol). The reaction mixture was stirred at 0° C. for 1 hr. On completion, the reaction mixture was quenched with saturated sodium thiosulfate (5 mL), then the organic layer was washed with saturated sodium hydrogen carbonate (5 mL×2) and brine (10 mL). Then the organics were dried over anhydrous sodium sulfate, filtered and concentrated in vacuo. The residue was purified by prep-HPLC (column: Phenomenex Luna C18 150*25 mm*10 um; mobile phase: [water(0.225% FA)-ACN]; B %: 43%-73%, 11.5 min) to give the title compound (20.0 mg, 44% yield) as a pink solid. ¹H NMR (400 MHz, DMSO-d₆) δ 10.24 (s, 1H), 9.65 (s, 1H), 8.45-8.31 (m, 3H), 8.17 (d, J=8.0 Hz, 1H), 7.93 (d, J=1.6 Hz, 1H), 7.03 (d, J=1.6 Hz, 1H), 3.93 (s, 3H), 3.30 (br s, 2H), 2.22-2.15 (m, 2H), 2.11 (br d, J=12.0 Hz, 2H), 1.98 (dq, J=2.8, 12.8 Hz, 2H), 1.44 (br dd, J=3.2, 13.2 Hz, 2H). LC-MS (ESI+) m/z 477.3 (M+H)⁺.

### 7-bromopyrrolo[1,2-b]pyridazine-3-carbonitrile (Intermediate BVT)

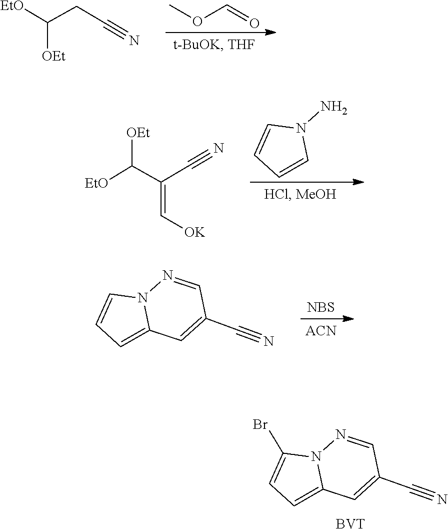

### Step 1—[(E)-2-cyano-3,3-diethoxy-prop-1-enoxy]potassium

To a solution of 3, 3-diethoxypropanenitrile (10.0 g, 69.8 mmol, CAS #2032-34-0) and methyl formate (5.45 g, 90.8, CAS #107-31-3) in THF (80 mL) was added 1M t-BuOK in THF (69.84 mL) slowly. The mixture was stirred at 20° C. for 2 hr. On completion, the mixture was added hexane (400 mL) and stirred for 20 min. Then the slurry was filtered and the filter cake washed with hexanes/THF (1:1) and dried at 60° C. in a vacuo to give the title compound (7 g, 48% yield) as a yellow solid. ¹H NMR (400 MHz, CD₃OD) δ 8.12 (s, 1H), 7.95 (s, 1H), 5.22 (s, 1H), 4.68 (s, 1H), 3.60-3.50 (m, J=7.0 Hz, 4H), 1.14 (t, J=7.0 Hz, 6H).

### Step 2—Pyrrolo[1,2-b]pyridazine-3-carbonitrile

To a solution of [(E)-2-cyano-3, 3-diethoxy-prop-1-enoxy]potassium (4 g, 19.1 mmol) was added HCl (12 M, 5.57 mL) slowly and stirred at 25° C. for 0.2 hr. Then the mixture was added pyrrol-1-amine (1.57 g, 19.1 mmol, CAS #765-39-9) in MeOH (20 mL). After the addition, the reaction mixture was stirred at 90° C. for 2 hrs. On completion, to the mixture was added NaHCO₃ (aq.) carefully until the resulting residue until bubbling stopped. Then the mixture was extracted with ethyl acetate (30 mL), the organic phase was washed with brine (20 mL×2), dried over anhydrous Na₂SO₄, filtered and concentrated in vacuo. The residue was purified by silica gel chromatography gel, Petroleum ether/Ethyl acetate=20/1) to give the title compound (2 g, 73% yield) as a yellow solid. ¹HNMR (400 MHz, CDCl₃) δ 8.14-8.11 (m, 2H), 7.94 (dd, J=1.6, 2.0 Hz, 1H), 7.05 (dd, J=2.8, 4.6 Hz, 1H), 6.85 (dd, J=1.2, 4.4 Hz, 1H).

### Step 3—7-bromopyrrolo[1,2-b]pyridazine-3-carbonitrile

To a solution of pyrrolo[1,2-b]pyridazine-3-carbonitrile (1.00 g, 6.99 mmol) in ACN (20 mL) was added NBS (1.24 g, 6.99 mmol), the mixture was stirred at 20° C. for 1 hr. On completion, the mixture was filtered and filter concentrated in vacuo to give a residue. The residue was triturated with Petroleum ether at 20° C. for 20 mins, the solid was collected by filtration. The residue was purified by reverse phase (0.1% FA condition) to give the title compound (1.40 g, 90% yield) as yellow solid. ¹H NMR (400 MHz, CDCl₃) δ 8.28 (d, J=1.6 Hz, 1H), 8.10 (d, J=2.0 Hz, 1H), 7.12 (d, J=4.8 Hz, 1H), 6.93 (d, J=4.6 Hz, 1H).

### 6-(3-cyanopyrrolo [1,2-b]pyridazin-7-yl)-N-(4-formylcyclohexyl)-4-(isopropylamino)pyridine-3-carboxamide (Intermediate BVU)

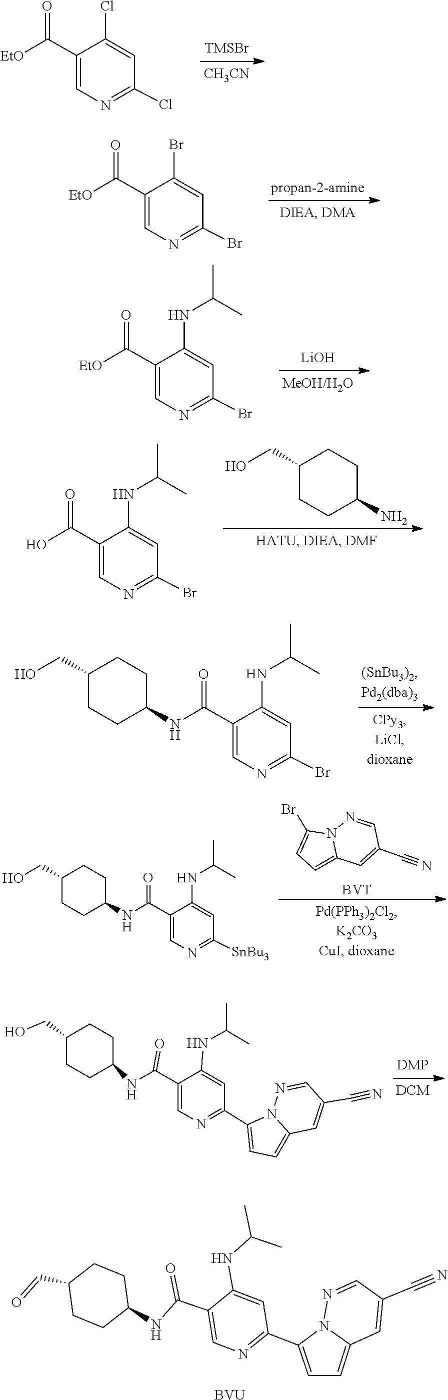

### Step 1—Ethyl 4,6-dibromopyridine-3-carboxylate

To a solution of ethyl 4, 6-dichloropyridine-3-carboxylate (10.0 g, 45.4 mmol) in ACN (200 mL) was added TMSBr (34.8 g, 227 mmol). The mixture was stirred at 80° C. for 16 hrs. On completion, the mixture was extracted with ethyl acetate (500 mL). The organic phase was washed with brine (100 mL×3), dried with anhydrous Na₂SO₄, filtered and concentrated in vacuo to give the title compound (13 g, 93% yield) as gray oil. ¹H NMR (400 MHz, CDCl₃) δ 8.77 (s, 1H), 7.86 (s, 1H), 4.44 (M, J=7.0 Hz, 2H), 1.43 (t, J=7.1 Hz, 3H); LC-MS (ESI⁺) m/z 309.8 (M+H)⁺.

### Step 2—Ethyl 6-bromo-4-(isopropylamino)pyridine-3-carboxylate

To a solution of ethyl 4, 6-dibromopyridine-3-carboxylate (13.0 g, 42.1 mmol) in DMA (50 mL) was added DIEA (27.2 g, 210 mmol) and propan-2-amine (2.49 g, 42.1 mmol). The mixture was stirred at 50° C. for 3 hrs. On completion, the reaction mixture was extracted with ethyl acetate (80 mL). The organic phase was washed with brine (40 mL), dried over anhydrous Na₂SO₄, filtered and the filtrated was concentrated in vacuo. The residue was purified by silica gel chromatography (silica gel, Petroleum ether/Ethyl acetate=10/1) to give the title compound (9.00 g, 74% yield) as yellow oil. ¹H NMR (400 MHz, CDCl₃) δ 8.53 (s, 1H), 8.01 (d, J=6.0 Hz, 1H), 6.64 (s, 1H), 4.26-4.23 (M, J=7.0 Hz, 2H), 3.61 (d, J=6.4, 13.2 Hz, 1H), 1.31 (t, J=7.2 Hz, 3H), 1.21 (d, J=6.4 Hz, 6H).

### Step 3—6-bromo-4-(isopropylamino)pyridine-3-carboxylic acid

To a solution of ethyl 6-bromo-4-(isopropylamino)pyridine-3-carboxylate (5.00 g, 17.4 mmol) in MeOH (25 mL) and H₂O (25 mL) was added LiOH·H₂O (3.65 g, 87.1 mmol). The mixture was then stirred at 50° C. for 16 hrs. On completion, the reaction mixture was added KHSO₄ (aq.) until the pH=5. Then the mixture was extracted with ethyl acetate (80 mL×2), and the organic phase was washed with brine (40 mL), dried with anhydrous Na₂SO₄, filtered and concentrated in vacuo to give the title compound (3.2 g, 71% yield) as a white solid. ¹H NMR (400 MHz, DMSO-d₆) δ 13.34 (s, 1H), 8.45 (s, 1H), 6.92 (s, 1H), 3.91-3.74 (m, 1H), 3.32 (s, 1H), 1.18 (d, J=6.3 Hz, 6H); LC-MS (ESI⁺) m/z 259.0 (M+H)⁺.

### Step 4—6-bromo-N-[4-(hydroxymethyl)cyclohexyl]-4-(isopropylamino)pyridine-3-carboxamide

To a solution of 6-bromo-4-(isopropylamino)pyridine-3-carboxylic acid (3.20 g, 12.4 mmol) in DMF (30 mL) was added HATU (5.64 g, 14.8 mmol), DIEA (4.79 g, 37.1 mmol) and (4-aminocyclohexyl)methanol (1.76 g, 13.6 mmol, CAS #1467-84-1). The mixture was then stirred at 25° C. for 1 hr. On completion, the reaction mixture was added into water (200 mL), the precipitate was collected by filtration to give the title compound (3.8 g, 83% yield) as white solid. ¹H NMR (400 MHz, DMSO-d₆) δ 8.40 (d, J=7.6 Hz, 1H), 8.31 (d, J=7.6 Hz, 1H), 6.80 (s, 1H), 4.39 (t, J=5.3 Hz, 1H), 3.78-3.62 (m, 2H), 3.22 (t, J=5.8 Hz, 2H), 2.07 (s, 2H), 1.87-1.74 (m, 4H), 1.29 (dd, J=2.4, 12.0 Hz, 2H), 1.15 (d, J=6.4 Hz, 6H), 1.01-0.88 (m, 2H); LC-MS (ESI⁺) m/z 372.1 (M+H)⁺.

### Step 5—N-[4-(hydroxymethyl)cyclohexyl]-4-(isopropylamino)-6-tributylstannyl-pyridine-3-carboxamide

To a solution of 6-bromo-N-((1r, 4r)-4-(hydroxymethyl)cyclohexyl)-4-(isopropylamino)nicotinamide (500 mg, 1.35 mmol) in dioxane (3 mL) was added LiCl (172 mg, 4.05 mmol), Pd₂(dba)₃ (123 mg, 135 umol), (SnBu₃)₂ (2.35 g, 4.05 mmol) and CPy₃ (37.9 mg, 135 umol). Then the mixture was stirred at 100° C. for 8 hr under nitrogen atmosphere. On completion, the reaction mixture was concentrated in vacuo to give the title compound (700 mg, 89% yield) as a yellow solid. LC-MS (ESI⁺) m/z 582.4 (M+H)⁺.

### Step 6—6-(3-cyanopyrrolo[1,2-b]pyridazin-7-yl)-N-[4-(hydroxymethyl)cyclohexyl]-4-(isopropylamino) pyridine-3-carboxamide

To a solution of N-((1r, 4r)-4-(hydroxymethyl)cyclohexyl)-4-(isopropylamino)-6-(tributylstannyl) nicotinamide (700 mg, 1.21 mmol) and 7-bromopyrrolo[1,2-b]pyridazine-3-carbonitrile (250 mg, 1.13 mmol, Intermediate BVT) in dioxane (15 mL) was added Pd(PPh₃)₂Cl₂ (84.7 mg, 121), CuI (23.0 mg, 121 umol) and K₂CO₃ (166 mg, 1.21 mmol). The mixture was then stirred at 110° C. under N₂ for 1 hr. On completion, the reaction mixture was filtered and the filtrate was concentrated in vacuo. The residue was purified by silica gel chromatography (100-200 mesh silica gel, Petroleum ether/Ethyl acetate=0/1) to give the title compound (300 mg, 58% yield) as yellow solid. ¹H NMR (400 MHz, DMSO-d₆) δ 8.83 (d, J=2.0 Hz, 1H), 8.70 (d, J=2.0 Hz, 1H), 8.67 (s, 1H), 8.46 (d, J=7.0 Hz, 1H), 8.26 (d, J=7.6 Hz, 1H), 8.05 (s, 1H), 7.82 (d, J=4.8 Hz, 1H), 7.10 (d, J=4.8 Hz, 1H), 4.40 (t, J=5.3 Hz, 1H), 3.81-3.68 (m, 2H), 3.23 (t, J=5.8 Hz, 2H), 1.90-1.84 (m, 2H), 1.79 (d, J=12.4 Hz, 2H), 1.39-1.29 (m, 3H), 1.27 (d, J=6.4 Hz, 6H), 1.03-0.92 (m, 2H).

### Step 7—6-(3-cyanopyrrolo [1,2-b]pyridazin-7-yl)-N-(4-formylcyclohexyl)-4-(isopropylamino)pyridine-3-carboxamide

To a solution of 6-(3-cyanopyrrolo [1,2-b]pyridazin-7-yl)-N-[4-(hydroxymethyl) cyclohexyl]-4-(isopropylamino) pyridine-3-carboxamide (210 mg, 486 umol) in DCM (5 mL) was added DMP (309 mg, 728 umol). The mixture was stirred at 20° C. for 4 hrs. On completion, the reaction mixture was diluted with DCM (40 mL), quenched with Na₂S₂O₃ (aq. 20 mL) and NaHCO₃ (aq. 20 mL). The mixture was stirred at 20° C. for 30 min, then the organic layer was washed with brine (3×10 mL). The organic phase was separated and dried with anhydrous Na₂SO₄, filtered and the filtrate was concentrated in vacuo. The residue was purified by silica gel chromatography (Petroleum ether/Ethyl acetate=3/1) to give the title compound (80 mg, 38% yield) as a yellow solid. LC-MS (ESI⁺) m/z 431.3 (M+H)⁺.

### 5-Chloro-N-[2-(4-formylcyclohexyl)-5-methoxy-1,3-benzothiazol-6-yl]pyridine-3-carboxamide (Intermediate BXF)

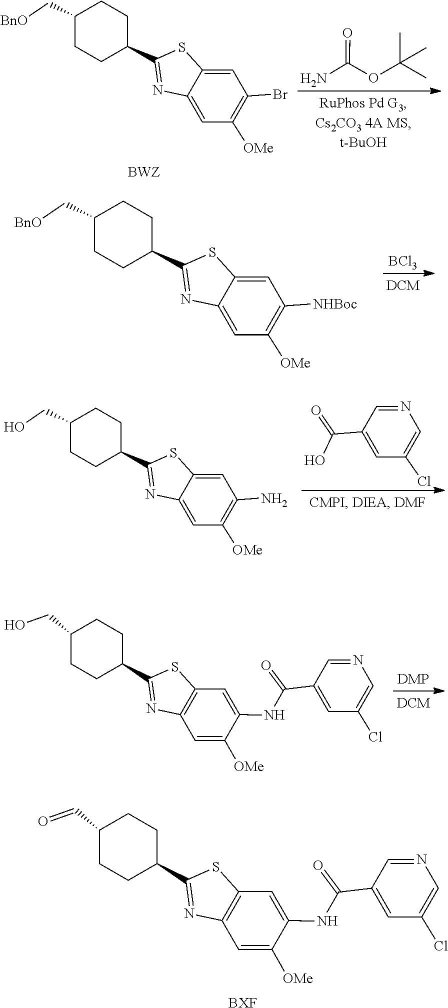

### Step 1—tert-butyl N-[2-[4-(benzyloxymethyl)cyclohexyl]-5-methoxy-1,3-benzothiazol-6-yl]carbamate

A mixture of 2-[4-(benzyloxymethyl) cyclohexyl]-6-bromo-5-methoxy-1,3-benzothiazole (2.00 g, 4.48 mmol, Intermediate BWZ), tert-butyl carbamate (524 mg, 4.48 mmol, CAS #4248-19-5), RuPhos Pd G3 (374 mg, 448 umol), Cs₂CO₃ (4.38 g, 13.4 mmol) and 4 Å molecular sieves (200 mg, 4.48 mmol) in t-BuOH (25 mL) was stirred at 80° C. for 12 hrs. On completion, the reaction mixture was filtered then the filtrate was diluted with water (30 mL), and extracted with EA (3×70 mL). The combined organic layer was dried over Na₂SO₄, filtered and concentrated in vacuo to give a residue. The residue was purified by reverse phase (0.1% FA condition) to give the title compound (1.06 g, 49% yield) as brown solid. ¹H NMR (400 MHz, DMSO-d₆) δ 8.27 (s, 1H), 8.07 (s, 1H), 7.54 (s, 1H), 7.38-7.35 (m, 1H), 7.34 (d, J=2.8 Hz, 2H), 7.32-7.26 (m, 2H), 4.46 (s, 2H), 3.88 (s, 3H), 3.33-3.28 (m, 4H), 3.05-2.96 (m, 1H), 2.15 (d, J=10.8 Hz, 2H), 1.89 (dd, J=2.8, 13.6 Hz, 2H), 1.70-1.63 (m, 1H), 1.46 (s, 9H), 1.20-1.13 (m, 2H). LC-MS (ESI⁺) m/z 483.3 (M+H)⁺.

### Step 2—[4-(6-Amino-5-methoxy-1,3-benzothiazol-2-yl)cyclohexyl]methanol

To a mixture of tert-butyl N-[2-[4-(benzyloxymethyl)cyclohexyl]-5-methoxy-1,3-benzothiazol-6-yl]carbamate (1.00 g, 2.07 mmol) in DCM (10 mL) was added BCl₃ (1 M, 6.22 mL). The reaction mixture was stirred at 0° C. for 1.5 hr. On completion, the reaction mixture was quenched with water (10 mL) and extracted with DCM (3×200 mL). The aqueous phase layer was filtered and concentrated in vacuo to give the title compound (1.00 g, 60% yield) as yellow solid. LC-MS (ESI⁺) m/z 293.1 (M+H)⁺.

### Step 3—5-Chloro-N-[2-[4-(hydroxymethyl)cyclohexyl]-5-methoxy-1,3-benzothiazol-6-yl]pyridine-3-carboxamide

To a mixture of 5-chloropyridine-3-carboxylic acid (86.2 mg, 547 umol, CAS #22620-27-5) in DMF (4 mL) was added CMPI (209 mg, 820 umol) and DIEA (265 mg, 2.05 mmol). Then a mixture of [4-(6-amino-5-methoxy-1, 3-benzothiazol-2-yl)cyclohexyl]methanol (200 mg, 684 umol) in DMF (4 mL) was added into the reaction mixture. The reaction mixture was then stirred at 25° C. for 2 hrs. On completion, the reaction mixture was quenched with water (0.05 mL) and concentrated in vacuo. The residue was purified by reverse phase (0.1% FA condition) to give the title compound (37.0 mg, 12% yield) as white solid. LC-MS (ESI⁺) m/z 432.1 (M+H)⁺.

### Step 4—5-Chloro-N-[2-(4-formylcyclohexyl)-5-methoxy-1,3-benzothiazol-6-yl]pyridine-3-carboxamide

To a mixture of 5-chloro-N-[2-[4-(hydroxymethyl) cyclohexyl]-5-methoxy-1, 3-benzothiazol-6-yl]pyridine-3-carboxamide (37.0 mg, 85.6 umol) in DCM (1 mL) was added DMP (54.5 mg, 128 umol). The reaction mixture was stirred at 25° C. for 1 hr. On completion, the reaction mixture was quenched with saturated Na₂S₂O₃ (3 mL) and saturated NaHCO₃ (3 mL) at 25° C., and then stirred for 30 minutes. The mixture was extracted with DCM (2×70 mL) then the combined organic layers was dried over Na₂SO₄, filtered and concentrated in vacuo to give the title compound (70.0 mg, 90% yield)) as white solid. LC-MS (ESI⁺) m/z 430.2 (M+H)⁺.

### 5-Cyano-N-[2-(4-formylcyclohexyl)-5-methoxy-1,3-benzothiazol-6-yl]pyridine-3-carboxamide (Intermediate BXG)

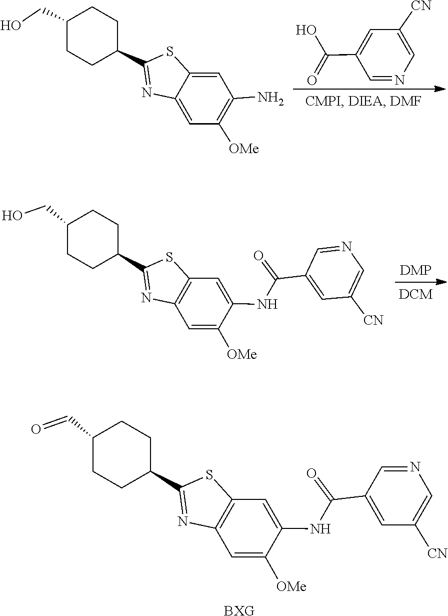

### Step 1—5-Cyano-N-[2-[4-(hydroxymethyl)cyclohexyl]-5-methoxy-1,3-benzothiazol-6-yl]pyridine-3-carboxamide

To a mixture of [4-(6-amino-5-methoxy-1,3-benzothiazol-2-yl)cyclohexyl]methanol (140 mg, 478 umol, synthesized via Steps 1-2 of Intermediate BXF) in DMF (1 mL) was added DIEA (61.8 mg, 478 umol. To a solution of 5-cyanopyridine-3-carboxylic acid (70.9 mg, 478 umol, CAS #887579-62-6) in DMF (1 mL) was added CMPI (122 mg, 478 umol) and DIEA (61.8 mg, 478 umol) and the mixture was stirred at 25° C. for 0.2 hour. Then the mixture was added to the reaction mixture solution dropwise. The reaction mixture was then stirred at 25° C. for 2 hours. On completion, the residue was diluted with water (30 mL) and extracted with EA (2×50 mL). The combined organic layer was dried over Na₂SO₄, filtered and concentrated in vacuo to give a residue. The residue was purified by reverse phase (0.1% FA condition) to give the title compound (50.0 mg, 24% yield) as white solid. LC-MS (ESI⁺) m/z 423.2 (M+H)⁺.

### Step 2—5-Cyano-N-[2-(4-formylcyclohexyl)-5-methoxy-1,3-benzothiazol-6-yl]pyridine-3-carboxamide

To a mixture of 5-cyano-N-[2-[4-(hydroxymethyl)cyclohexyl]-5-methoxy-1,3-benzothiazol-6-yl]pyridine-3-carboxamide (40.0 mg, 94.6 umol) in DCM (1 mL) was added DMP (52.2 mg, 123 umol). The reaction mixture was then stirred at 25° C. for 1 hour. On completion, the reaction mixture was quenched by saturated Na₂S₂O₃ (1 mL) and saturated NaHCO₃ (1 mL) at 25° C., and then stirred for 30 minutes. The residue was diluted with water (15 mL) and extracted with DCM (2×20 mL). The combined organic layer was dried over Na₂SO₄, filtered and concentrated in vacuo to give the title compound (28.0 mg, 70% yield) as yellow solid. LC-MS (ESI⁺) m/z 421.2 (M+H)⁺.

### 4-[5-[[6-(Trifluoromethyl)pyridine-2-carbonyl]amino]pyrazolo[3,4-c]pyridin-2-yl]cyclohexanecarboxylic acid (Intermediate BXH)

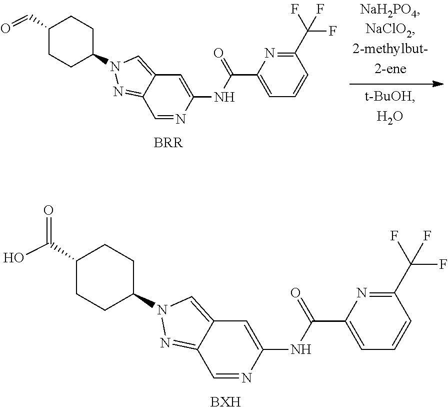

To a mixture of N-[2-(4-formylcyclohexyl)pyrazolo[3,4-c]pyridin-5-yl]-6-(trifluoromethyl)pyridine-2-carboxamide (100 mg, 239 umol, Intermediate BRR), NaH₂PO₄ (28.7 mg, 239 umol) and 2-methylbut-2-ene (67.1 mg, 958 umol) in t-BuOH (3 mL) and H₂O (1 mL) was added sodium chlorite (65.0 mg, 718 umol) at 0° C. The mixture was then stirred at 25° C. for 4 hrs. On completion, the mixture was adjusted pH to 2-3 with HCl (0.5 M), then concentrated under reduced pressure to give a residue. The residue was diluted with DCM (4 mL) and extracted with DCM 30 mL (15 mL×2). The combined organic layers were washed with H₂O 30 mL (15 mL×2), dried over Na₂SO₄, then concentrated under reduced pressure to give the title compound (100 mg, 96% yield) as white solid. LC-MS (ESI⁺) m/z 434.4 (M+H)⁺.

### N-[6-cyano-2-(4-formylcyclohexyl)indazol-5-yl]-6-(trifluoromethyl)pyridine-2-carboxamide (Intermediate BXI)

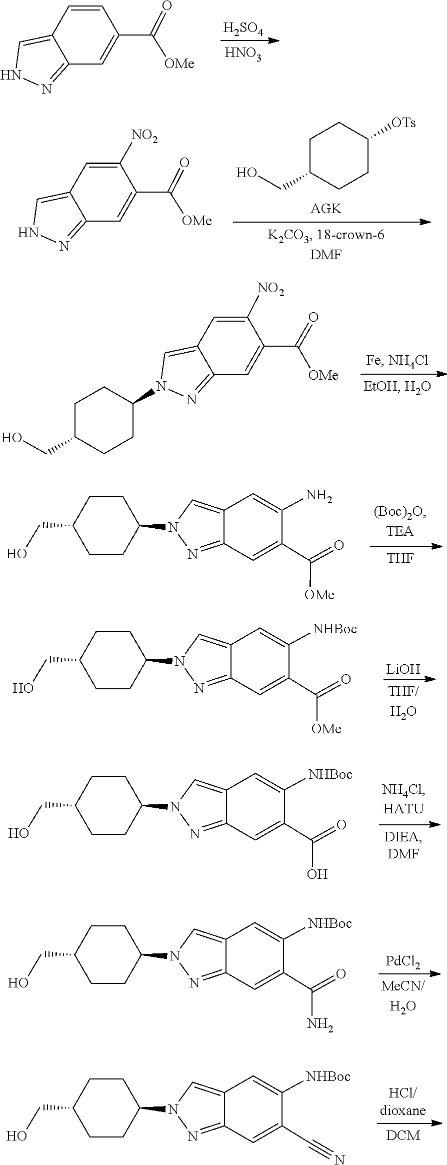

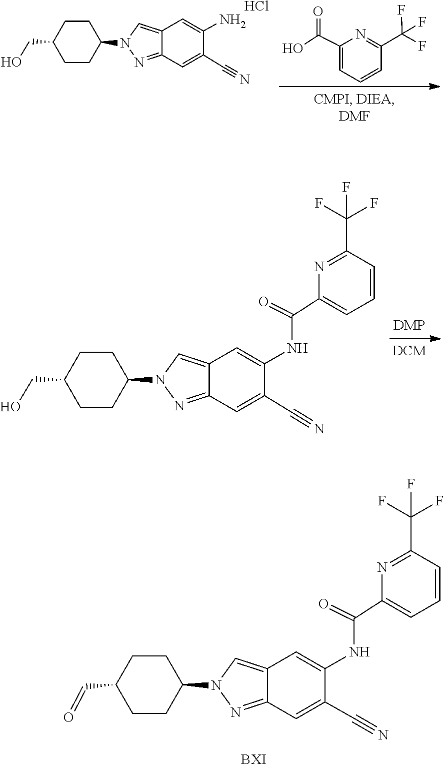

### Step 1—Methyl 5-nitro-2H-indazole-6-carboxylate

To a solution of methyl 2H-indazole-6-carboxylate (30.0 g, 170 mmol, CAS #170487-40-8) in H₂SO₄ (200 mL) was added a solution of HNO₃ (45.9 g, 511 mmol, 70% solution) in H₂SO₄ (40 mL) dropwise at 0-10° C. The reaction mixture was stirred at 0° C. for 30 minutes. On completion, the mixture was poured into ice water (1.5 L), stirred and filtered. The filter cake was washed with water (4×100 mL), then dried in vacuo to give the title compound (34.0 g, 90% yield) as a yellow solid. ¹H NMR (400 MHz, DMSO-d₆) δ 8.69 (s, 1H), 8.45 (s, 1H), 7.96 (s, 1H), 3.87 (s, 3H)

### Step 2—Methyl 2-[4-(hydroxymethyl)cyclohexyl]-5-nitro-indazole-6-carboxylate

To a solution of methyl 5-nitro-2H-indazole-6-carboxylate (15.0 g, 67.8 mmol) and [4-(hydroxymethyl)cyclohexyl]4-methylbenzenesulfonate (48.2 g, 169 mmol, Intermediate AGK) in DMF (300 mL) was added K₂CO₃ (23.4 g, 169 mmol), 18-CROWN-6 (1.79 g, 6.78 mmol) and 4 Å molecular sieves (2 g). The reaction mixture was stirred at 80° C. for 2 days. On completion, the mixture was concentrated in vacuo, then diluted with water (1 L), and extracted with EA (2×300 mL). The organic layer was washed with brine (200 mL), then concentrated in vacuo. The residue was purified by silica gel chromatography (SiO₂) to give the title compound (5.00 g, 22% yield) as a yellow solid. ¹H NMR (400 MHz, CDCl₃) δ 8.42 (s, 1H), 8.23 (d, J=0.6 Hz, 1H), 8.03 (s, 1H), 4.55-4.40 (m, H), 3.93 (s, 3H), 3.57 (t, J=5.2 Hz, 2H), 2.44-2.31 (m, 2H), 2.14-1.95 (m, 4H), 1.68-1.62 (m, 1H), 1.55 (t, J=4.8 Hz, 1H), 1.35-1.24 (m, 2H).

### Step 3—Methyl 5-amino-2-[4-(hydroxymethyl)cyclohexyl]indazole-6-carboxylate

To a solution of methyl 2-[4-(hydroxymethyl)cyclohexyl]-5-nitro-indazole-6-carboxylate (4.94 g, 14.8 mmol) in a mixed solvent of EtOH (70 mL) and H₂O (20 mL) was added Fe (8.28 g, 148 mmol) and NH₄Cl (7.93 g, 148 mmol). The reaction mixture was stirred at 70° C. for 1 hr. On completion, the mixture was diluted with water (200 mL), then extracted with EA (2×200 mL). The organic layer was washed with brine (200 mL), dried with Na₂SO₄, filtered and the filtrate was concentrated in vacuo to give the title compound (3.60 g, 80% yield) as a yellow solid. LC-MS (ESI⁺) m/z 304.1 (M+H)⁺.

### Step 4—Methyl 5-(tert-butoxycarbonylamino)-2-[4-(hydroxymethyl)cyclohexyl]indazole-6-carboxylate

To a solution of methyl 5-amino-2-[4-(hydroxymethyl)cyclohexyl]indazole-6-carboxylate (520 mg, 1.71 mmol) in THF (5 mL) was added TEA (260 mg, 2.57 mmol) and (Boc)₂O (411 mg, 1.89 mmol) dropwise. Then the mixture was stirred at 60° C. for 4 hrs. On completion, the mixture was quenched with H₂O (5 mL), then extracted with EA (5 mL×3). The combined organic phase was dried over anhydrous sodium sulfate, filtered and concentrated to give a residue. The residue was purified by column chromatography (SiO₂, PE/EA=100:1 to 50:1) to give the title compound (450 mg, 65% yield) as yellow solid. ¹H NMR (400 MHz, CDCl₃) δ 10.05 (s, 1H), 8.61-8.49 (m, 2H), 7.87 (s, 1H), 4.46-4.37 (m, 1H), 3.97 (s, 3H), 3.58 (d, J=6.0 Hz, 2H), 2.39-2.32 (m, 2H), 2.12-1.94 (m, 5H), 1.75-1.63 (m, 1H), 1.56 (s, 9H), 1.30-1.24 (m, 2H).

### Step 5—5-(Tert-butoxycarbonylamino)-2-[4-(hydroxymethyl)cyclohexyl]indazole-6-carboxylic acid

To a solution of methyl 5-(tert-butoxycarbonylamino)-2-[4-(hydroxymethyl)cyclohexyl]indazole-6-carboxylate (400 mg, 991 umol) in THF (2 mL) and H₂O (0.5 mL) was added LiOH·H₂O (124 mg, 2.97 mmol). The mixture was then stirred at 50° C. for 4 hrs. On completion, the mixture was concentrated in vacuo, then diluted with H₂O (8 mL), and adjusted to pH of 4 using 0.5 M HCl aqueous to precipitate a solid. The solid was filtered, and the filter cake was dried in vacuo to give the title compound (377 mg, 97% yield) as yellow solid. LC-MS (ESI⁺) m/z 390.2 (M+H)⁺.

### Step 6—Tert-butyl N-[6-carbamoyl-2-[4-(hydroxymethyl)cyclohexyl]indazol-5-yl]carbamate

To a solution of 5-(tert-butoxycarbonylamino)-2-[4-(hydroxymethyl)cyclohexyl]indazole-6-carboxylic acid (370 mg, 950 umol) in DMF (5 mL) was added NH₄Cl (203 mg, 3.80 mmol), HATU (433 mg, 1.14 mmol) and DIEA (245 mg, 1.90 mmol). The mixture was then stirred at 20° C. for 2 hrs. On completion, the mixture was quenched with H₂O (10 mL), and extracted with EA (20 mL×3). The combined organic phase was dried over anhydrous sodium sulfate, filtered and concentrated to give the title compound (320 mg, 86% yield) as yellow solid. ¹H NMR (400 MHz, DMSO-d₆) δ 10.32 (s, 1H), 8.38-8.27 (m, 3H), 8.10 (s, 1H), 7.70 (s, 1H), 4.54-4.38 (m, 2H), 3.68-3.57 (m, 1H), 3.29 (t, J=5.6 Hz, 2H), 3.20-3.11 (m, 1H), 2.39-2.20 (m, 2H), 1.89 (s, 2H), 1.48 (s, 9H), 1.22-1.09 (m, 3H).

### Step 7—Tert-butyl N-[6-cyano-2-[4-(hydroxymethyl)cyclohexyl]indazol-5-yl]carbamate

To a solution of tert-butyl N-[6-carbamoyl-2-[4-(hydroxymethyl)cyclohexyl]indazol-5-yl]carbamate (300 mg, 772 umol) in MeCN (1 mL) and H₂O (1 mL) was added PdCl₂ (13.6 mg, 77.2 umol). Then the mixture was then stirred at 55° C. for 2 hrs. On completion, the residue was filtered and the filtrate was purified by reverse phase (0.1% FA condition) to give the title compound (210 mg, 73% yield) as brown solid. ¹H NMR (400 MHz, DMSO-d6) δ 9.14-9.06 (m, 1H), 8.53 (s, 1H), 8.29 (s, 1H), 7.65 (s, 1H), 4.57-4.46 (m, 2H), 2.39-2.18 (m, 2H), 1.97-1.83 (m, 6H), 1.46 (s, 9H), 1.23-1.12 (m, 3H).

### Step 8—5-Amino-2-[4-(hydroxymethyl)cyclohexyl]indazole-6-carbonitrile

Tert-butyl N-[6-cyano-2-[4-(hydroxymethyl)cyclohexyl]indazol-5-yl]carbamate (150 mg, 404 umol) was dissolved in HCl/dioxane (4 M, 3 mL). The mixture was then stirred at 20° C. for 1 hrs. On completion, the mixture was concentrated in vacuo to give the title compound (124 mg, 99% yield, HCl) as yellow solid. LC-MS (ESI⁺) m/z 271.2 (M+H)⁺.

### Step 9—N-[6-cyano-2-[4-(hydroxymethyl)cyclohexyl]indazol-5-yl]-6-(trifluoromethyl)pyridine-2-carboxamide

To a solution of 6-(trifluoromethyl)pyridine-2-carboxylic acid (36.4 mg, 190 umol, CAS #131747-42-7) in DMF (0.5 mL) was added CMPI (48.7 mg, 190 umol) and DIEA (54.7 mg, 423 umol). Then 5-amino-2-[4-(hydroxymethyl)cyclohexyl]indazole-6-carbonitrile (65.0 mg, 211 umol, HCl) in DMF (0.5 mL) was added dropwise. The mixture was then stirred at 20° C. for 16 hrs. On completion, the mixture was quenched with H₂O (0.5 mL) and purified by pre-HPLC (column: Phenomenex Luna C18 150*25 mm*10 um; mobile phase: [water (0.225% FA)-ACN]; B %: 38%-68%, 11.5 min.) to give the title compound (48.0 mg, 51% yield) as white solid. ¹H NMR (400 MHz, DMSO-d₆) δ 10.64 (s, 1H), 8.64 (s, 1H), 8.47-8.36 (m, 3H), 8.27-8.20 (m, 2H), 4.62-4.47 (m, 2H), 3.31-3.28 (m, 2H), 2.25-2.12 (m, 2H), 2.01-1.89 (m, 4H), 1.62-1.41 (m, 1H), 1.25-1.13 (m, 2H).

### Step 10—N-[6-cyano-2-(4-formylcyclohexyl)indazol-5-yl]-6-(trifluoromethyl)pyridine-2-carboxamide

To a solution of N-[6-cyano-2-[4-(hydroxymethyl)cyclohexyl]indazol-5-yl]-6-(trifluoromethyl) pyridine-2-carboxamide (40.0 mg, 90.2 umol) in DCM (2 mL) was added DMP (45.9 mg, 108 umol). The mixture was then stirred at 20° C. for 1 hr. On completion, the mixture was quenched with Na₂S₂O₃ aqueous, and extracted with DCM (5 mL×3). The combined organic phase was washed with NaHCO₃ aqueous, water, brine, dried over anhydrous Na₂SO₄, filtered and concentrated in vacuo to give the title compound (39.0 mg, 97% yield) as off-white solid. LC-MS (ESI⁺) m/z 464.1 (M+H)⁺.

### 5-chloro-6-methoxy-pyridine-3-carboxylic acid (CAS #884494-85-3) (Intermediate BXJ)

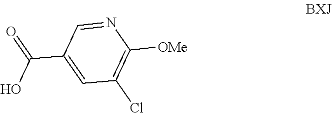 [SMILES: COc1ncc(C(=O)O)cc1Cl]

### 5-chloropyridine-3-carboxylic acid (CAS #22620-27-5) (Intermediate BXK)

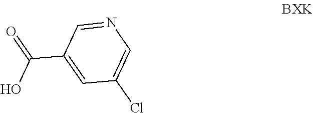

### 1-(8-Piperazin-1-yl-4-isoquinolyl)hexahydropyrimidine-2,4-dione (Intermediate BXL)

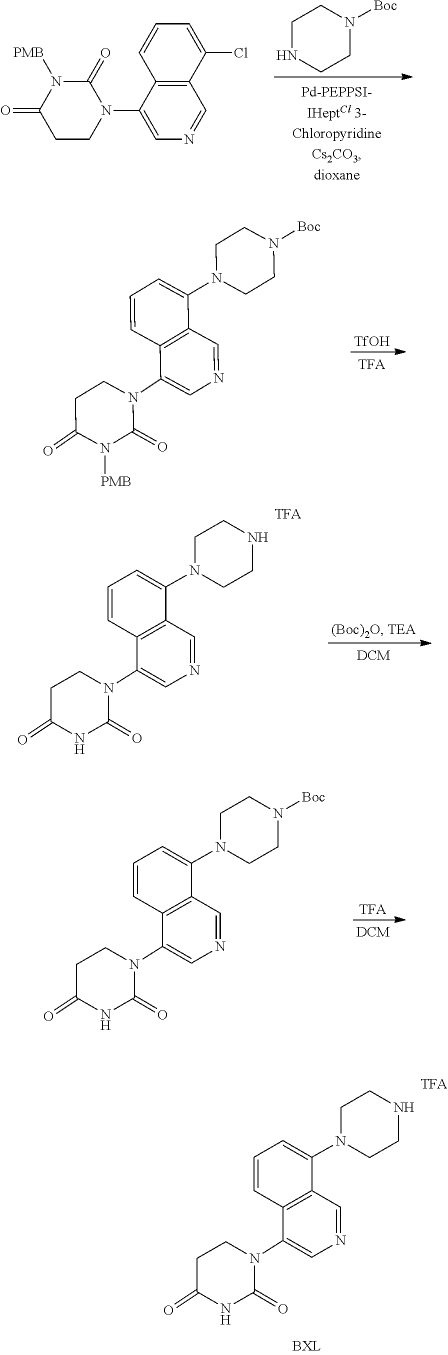

### Step 1—Tert-butyl 4-[4-[3-[(4-methoxyphenyl)methyl]-2,4-dioxo-hexahydropyrimidin-1-yl]-8-isoquinolyl]piperazine-1-carboxylate

To a solution of 1-(8-chloro-4-isoquinolyl)-3-[(4-methoxyphenyl)methyl]hexahydropyrimidine-2,4-dione (900 mg, 2.27 mmol, synthesized via Steps 1-2 of Intermediate BSL) and tert-butyl piperazine-1-carboxylate (508 mg, 2.73 mmol, CAS #143238-38-4) in dioxane (15 mL) was added Pd-PEPPSI-IHeptCl 3-Chloropyridine (221 mg, 227 umol) and Cs₂CO₃ (1.48 g, 4.55 mmol), then the mixture was stirred at 80° C. for 8 hours. On completion, the mixture was filtered and concentrated in vacuo to give the title compound (1.20 g, 96% yield) as a brown solid. ¹H NMR (400 MHz, CDCl₃-d) δ 9.52 (s, 1H), 8.40 (s, 1H), 7.56 (t, J=8.0 Hz, 1H), 7.37 (d, J=8.8 Hz, 2H), 7.23-7.19 (m, 1H), 7.09 (d, J=7.6 Hz, 1H), 6.78 (d, J=8.4 Hz, 2H), 4.94 (s, 2H), 3.85-3.75 (m, 2H), 3.74 (s, 3H), 3.70-3.64 (m, J=6.4, 12.4 Hz, 4H), 3.34-3.31 (m, 1H), 3.13-3.07 (m, 2H), 2.97-2.93 (m, 2H), 2.76-2.73 (m, 1H), 1.45 (s, 9H). LC-MS (ESI⁺) m/z 546.6 (M+H)⁺.

### Step 2—1-(8-Piperazin-1-yl-4-isoquinolyl)hexahydropyrimidine-2,4-dione

To a solution of tert-butyl 4-[4-[3-[(4-methoxyphenyl)methyl]-2,4-dioxo-hexahydropyrimidin-1-yl]-8-isoquinolyl]piperazine-1-carboxylate (600 mg, 1.10 mmol) in TFA (6 mL) was added TfOH (1 mL), then the mixture was stirred at 70° C. for 2 hours. On completion, the mixture was concentrated in vacuo to give the title compound (480 mg, 99% yield, TFA) as a black brown solid. LC-MS (ESI⁺) m/z 326.1 (M+H)⁺.

### Step 3—Tert-butyl 4-[4-(2,4-dioxohexahydropyrimidin-1-yl)-8-isoquinolyl]piperazine-1-carboxylate

To a solution of 1-(8-piperazin-1-yl-4-isoquinolyl)hexahydropyrimidine-2,4-dione (480 mg, 1.09 mmol, TFA) in DCM (5 mL) was added TEA (304 uL, 2.18 mmol) and Boc₂O (357 mg, 1.64 mmol), then the mixture was stirred at 25° C. for 10 hours. On completion, the mixture was diluted with DCM (300 mL) and extracted with water (200 mL×3). The combined organic layer was dried over anhydrous Na₂SO₄, filtered and concentrated in vacuo to give the residue. The residue was purified by reverse-phase (0.1% FA condition) to give the title compound (150 mg, 32% yield) as a yellow solid. ¹H NMR (400 MHz, CDCl₃-d) δ 9.61 (s, 1H), 8.52 (s, 1H), 8.26 (s, 1H), 7.69 (t, J=8.0 Hz, 1H), 7.47 (d, J=8.4 Hz, 1H), 7.18 (d, J=7.6 Hz, 1H), 4.03-3.91 (m, 1H), 3.90-3.77 (m, 2H), 3.73 (s, 3H), 3.23-3.02 (m, 4H), 3.01-2.88 (m, 2H), 1.51 (s, 9H). LC-MS (ESI⁺) m/z 426.1 (M+H)⁺.

### Step 4—1-(8-Piperazin-1-yl-4-isoquinolyl)hexahydropyrimidine-2,4-dione

To a solution of tert-butyl 4-[4-(2,4-dioxohexahydropyrimidin-1-yl)-8-isoquinolyl]piperazine-1-carboxylate (80.0 mg, 188 umol) in DCM (2 mL) was added TFA (0.5 mL, 6.75 mmol), then the mixture was stirred at 25° C. for 1 hour. On completion, the mixture was concentrated in vacuo to give the title compound (80.0 mg, 96% yield, TFA) as a brown solid. LC-MS (ESI⁺) m/z 326.1 (M+H)⁺.

### 3-(3-methyl-2-oxo-4-(3-(piperidin-4-yloxy)prop-1-yn-1-yl)-2,3-dihydro-1H-benzo[d]imidazol-1-yl)piperidine-2,6-dione (Intermediate BXN)

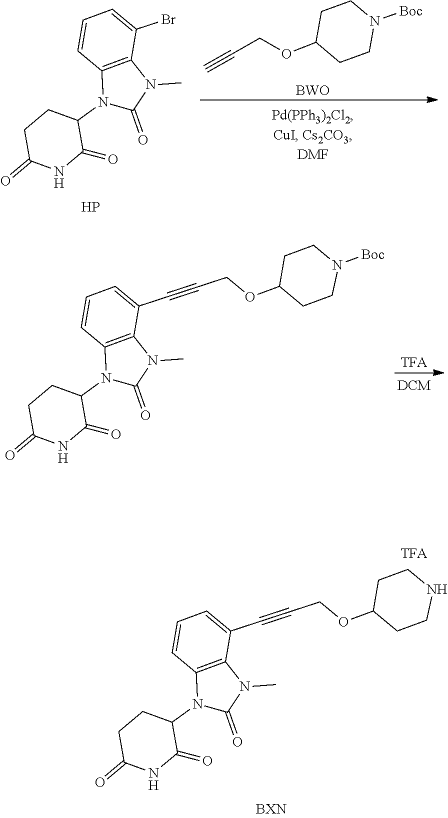

### Step 1—Tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate

A mixture of 3-(4-bromo-3-methyl-2-oxo-benzimidazol-1-yl)piperidine-2,6-dione (1.70 g, 5.01 mmol, Intermediate HP), tert-butyl 4-prop-2-ynoxypiperidine-1-carboxylate (1 g, 4.18 mmol, Intermediate BWO), Pd(PPh₃)₂Cl₂ (293 mg, 417 umol,), Cs₂CO₃ (2.72 g, 8.36 mmol) and CuI (79.5 mg, 417 umol) in DMF (25 mL) was degassed and purged with N₂ for 3 times. Then the mixture was stirred at 80° C. for 16 hrs under N₂ atmosphere. On completion, the reaction mixture was diluted by EtOAc (50 mL), filtered and the filtrate was concentrated in vacuo. The residue was purified by reverse phase (0.1% FA condition) to give the title compound (900 mg, 43% yield) as yellow solid. ¹H NMR (400 MHz, DMSO-d₆) δ 11.12 (s, 1H), 7.17 (d, J=7.9 Hz, 1H), 7.14-7.09 (m, 1H), 7.06-7.00 (m, 1H), 5.40 (dd, J=5.6, 12.8 Hz, 1H), 4.50 (s, 2H), 3.75-3.71 (m, 1H), 3.68-3.60 (m, 5H), 3.04 (t, J=9.6 Hz, 2H), 2.95-2.83 (m, 1H), 2.75-2.60 (m, 2H), 2.06-1.99 (m, 1H), 1.89-1.79 (m, 2H), 1.41 (d, J=4.0 Hz, 1H), 1.39 (s, 9H), 1.37-1.33 (m, 1H).

### Step 2—3-(3-methyl-2-oxo-4-(3-(piperidin-4-yloxy)prop-1-yn-1-yl)-2,3-dihydro-1H-benzo[d]imidazol-1-yl)piperidine-2,6-dione

To a solution of tert-butyl 4-[3-[1-(2,6-dioxo-3-piperidyl)-3-methyl-2-oxo-benzimidazol-4-yl]prop-2-ynoxy]piperidine-1-carboxylate (50 mg, 100 umol) in DCM (1 mL) was added TFA (0.3 mL). The reaction mixture was then stirred at 25° C. for 1 hr. On completion, the reaction mixture was concentrated in vacuo to give the title compound (39 mg, 97% yield) as brown oil. LC-MS (ESI⁺) m/z 397.1 (M+H)⁺.

### (Intermediate BXO)

### 1-[8-[1-[[4-(5-Aminoindazol-2-yl)cyclohexyl]methyl]-4-piperidyl]-4-isoquinolyl]hexahydro pyrimidine-2,4-dione (Intermediate BXP)

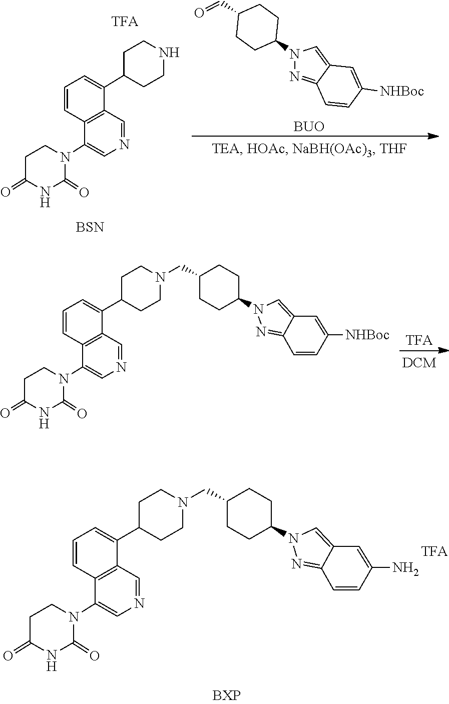

### Step 1—Tert-butyl N-[2-[4-[[4-[4-(2,4-dioxohexahydropyrimidin-1-yl)-8-isoquinolyl]-1-piperidyl] methyl]cyclohexyl]indazol-5-yl]carbamate

To a mixture of 1-[8-(4-piperidyl)-4-isoquinolyl]hexahydropyrimidine-2,4-dione (180 mg, 554 umol, Intermediate BSN) in DMF (5 mL) was added TEA (56.1 mg, 554 umol) until pH stabilized at 8. The mixture was stirred at 25° C. for 0.25 hr, then AcOH (33.3 mg, 554 umol) was added at −15° C. until the pH stabilized at 5-6. Subsequently, tert-butyl N-[2-(4-formylcyclohexyl)indazol-5-yl]carbamate (190 mg, 554 umol, Intermediate BUO) was added and stirred for 0.5 hr. After that, NaBH(OAc)₃ (152 mg, 721 umol) was added in one portion. The resulting reaction mixture was stirred at −15° C. for 1 hr. On completion, the mixture was quenched with H₂O (0.5 mL) and purified by reversed-phase (0.10% FA condition) to give the title compound (140 mg, 39% yield) as pink solid. ¹HNMR (400 MHz, DMSO-d₆) δ 10.55 (s, 1H), 9.63 (s, 1H), 9.21 (s, 1H), 8.58 (s, 1H), 8.16 (s, 1H), 7.95-7.77 (m, 3H), 7.69-7.64 (m, 1H), 7.50-7.41 (m, 1H), 7.24-7.16 (m, 1H), 4.50-4.33 (m, 1H), 3.98-3.89 (m, 1H), 3.69-3.60 (m, 1H), 3.17 (d, J=10.4 Hz, 2H), 2.92-2.88 (m, 1H), 2.47-2.41 (m, 3H), 2.21-2.11 (m, 2H), 2.06-1.88 (m, 8H), 1.81-1.70 (m, 1H), 1.49 (s, 9H), 1.17 (t, J=7.2 Hz, 5H); LC-MS (ESI⁺) m/z 652.4 (M+H)⁺.

### Step 2—1-[8-[1-[[4-(5-Aminoindazol-2-yl)cyclohexyl]methyl]-4-piperidyl]-4-isoquinolyl]hexahydro pyrimidine-2,4-dione

To a solution of tert-butyl N-[2-[4-[[4-[4-(2,4-dioxohexahydropyrimidin-1-yl)-8-isoquinolyl]-1-piperidyl]methyl]cyclohexyl]indazol-5-yl]carbamate (48.0 mg, 73.6 umol) in CH₂Cl₂ (2 mL) was added TFA (770 mg, 6.75 mmol). The mixture was then stirred at 20° C. for 1 hr. On completion, the mixture was concentrated to give the title compound (46.0 mg, 93% yield, TFA) as pink solid. LC-MS (ESI⁺) m/z 552.40 (M+H)⁺.

### 3-[4-(2,7-Diazaspiro[3.5]nonan-7-yl)-3-methyl-2-oxo-benzimidazol-1-yl]piperidine-2,6-dione (Intermediate BXQ)

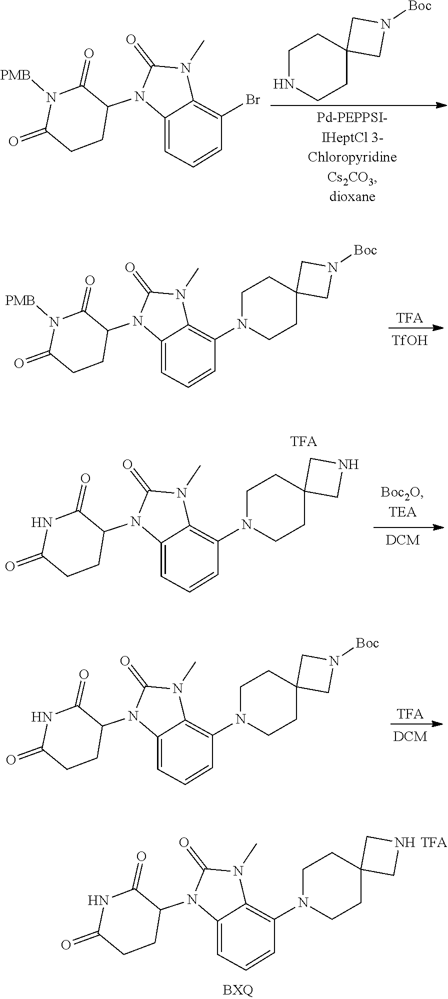

### Step 1—Tert-butyl 7-[1-[1-[(4-methoxyphenyl)methyl]-2,6-dioxo-3-piperidyl]-3-methyl-2-oxo-benzimidazol-4-yl]-2,7-diazaspiro[3.5]nonane-2-carboxylate

To a solution of 3-(4-bromo-3-methyl-2-oxo-benzimidazol-1-yl)-1-[(4-methoxyphenyl)methyl]piperidine-2,6-dione (1.00 g, 2.18 mmol, synthesized via Steps 1-4 of Intermediate HP) and tert-butyl2,7-diazaspiro[3.5]nonane-2-carboxylate (592 mg, 2.62 mmol, CAS #236406-55-6) in dioxane (20 mL) was added Cs₂CO₃ (1.42 g, 4.36 mmol) and Pd-PEPPSI-IHeptCl 3-Chloropyridine (212 mg, 218 umol). Then the mixture was stirred at 100° C. for 12 hours. On completion, the mixture was filtered and concentrated in vacuo to give the residue. The residue was purified by reverse-phase (0.1% FA condition) to give the title compound (380 mg, 28% yield) as a yellow solid. ¹H NMR (400 MHz, CDCl₃-d) δ 7.37 (d, J=8.4 Hz, 2H), 6.88-6.81 (m, 4H), 6.29 (dd, J=1.6, 6.8 Hz, 1H), 5.20 (dd, J=5.6, 13.2 Hz, 1H), 5.02-4.91 (m, 2H), 3.79 (s, 3H), 3.75 (s, 5H), 3.67 (s, 2H), 3.15-2.94 (m, 3H), 2.88-2.77 (m, 1H), 2.74-2.55 (m, 3H), 2.17-2.16 (m, 1H), 1.95 (s, 4H), 1.47 (s, 9H); LC-MS (ESI⁺) m/z 604.5 (M+H)⁺.

### Step 2—3-[4-(2,7-Diazaspiro[3.5]nonan-7-yl)-3-methyl-2-oxo-benzimidazol-1-yl]piperidine-2,6-dione

To a solution of tert-butyl 7-[1-[1-[(4-methoxyphenyl)methyl]-2,6-dioxo-3-piperidyl]-3-methyl-2-oxo-benzimidazol-4-yl]-2,7-diazaspiro[3.5]nonane-2-carboxylate (200 mg, 331 umol) in TFA (2 mL) was added TfOH (0.2 mL), then the mixture was stirred at 60° C. for 2 hours. On completion, the mixture was concentrated in vacuo to give the title compound (150 mg, 91% yield, TFA) as a brown solid. LC-MS (ESI⁺) m/z 384.2 (M+H)⁺.

### Step 3—Tert-butyl 7-[1-(2,6-dioxo-3-piperidyl)-3-methyl-2-oxo-benzimidazol-4-yl]-2,7-diazaspiro [3.5]nonane-2-carboxylate

To a solution of 3-[4-(2,7-diazaspiro[3.5]nonan-7-yl)-3-methyl-2-oxo-benzimidazol-1-yl]piperidine-2,6-dione (150 mg, 301 umol, TFA) in DCM (4 mL) was added TEA (83.9 uL, 603 umol) and Boc₂O (98.7 mg, 452 umol), then the mixture was stirred at 25° C. for 10 hours. On completion, the mixture was diluted with DCM (100 mL) and extracted with water (50 mL×3). The combined organic layer was dried over anhydrous Na₂SO₄, filtered and concentrated in vacuo to give the title compound (140 mg, 96% yield) as a brown solid. ¹H NMR (400 MHz, CDCl₃-d) δ 8.45 (s, 1H), 7.01-6.96 (m, 1H), 6.87 (d, J=8.0 Hz, 1H), 6.62-6.57 (m, 1H), 5.21 (dd, J=5.2, 12.4 Hz, 1H), 3.75 (s, 3H), 3.67 (s, 2H), 3.14-3.07 (m, J=7.6, 14.8 Hz, 3H), 2.94-2.88 (m, 1H), 2.85-2.80 (m, J=4.8, 13.2 Hz, 1H), 2.75-2.65 (m, 3H), 2.25-2.15 (m, 2H), 1.94 (s, 4H), 1.46 (s, 9H). LC-MS (ESI⁺) m/z 484.3 (M+H)⁺.

### Step 4—3-[4-(2,7-Diazaspiro[3.5]nonan-7-yl)-3-methyl-2-oxo-benzimidazol-1-yl]piperidine-2,6-dione

To a solution of tert-butyl 7-[1-(2,6-dioxo-3-piperidyl)-3-methyl-2-oxo-benzimidazol-4-yl]-2,7-diazaspiro[3.5]nonane-2-carboxylate (80.0 mg, 165 umol) in DCM (2 mL) was added TFA (1 mL, 13.5 mmol), then the mixture was stirred at 25° C. for 1 hour. On completion, the mixture was concentrated in vacuo to give the title compound (80.0 mg, 97% yield, TFA) as a black-brown solid. LC-MS (ESI⁺) m/z 384.2 (M+H)⁺.

### 3-[4-(3,9-Diazaspiro[5.5]undecan-3-yl)-3-methyl-2-oxo-benzimidazol-1-yl]piperidine-2,6-dione (Intermediate BXR)

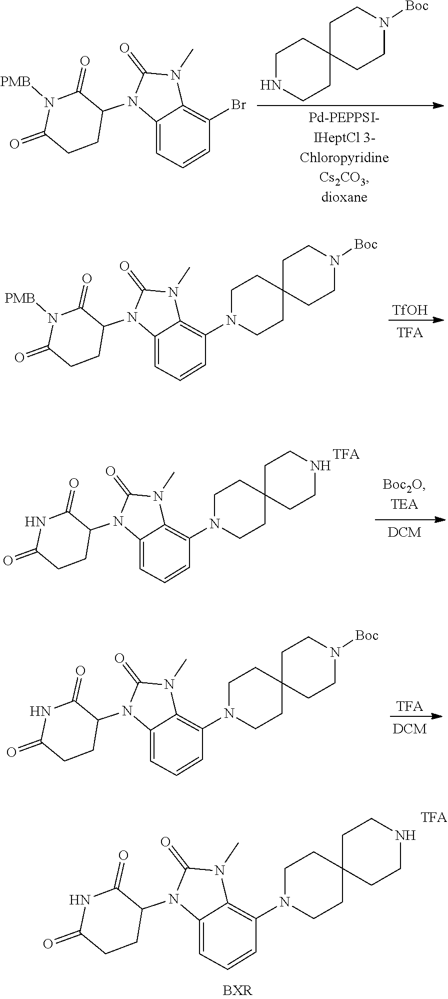

### Step 1—3-[4-(3,9-Diazaspiro[5.5]undecan-3-yl)-3-methyl-2-oxo-benzimidazol-1-yl]piperidine-2,6-dione

To a solution of 3-(4-bromo-3-methyl-2-oxo-benzimidazol-1-yl)-1-[(4-methoxyphenyl)methyl]piperidine-2,6-dione (1.00 g, 2.18 mmol, synthesized via Steps 1-4 of Intermediate HP) and tert-butyl-3,9-diazaspiro[5.5]undecane-3-carboxylate (666 mg, 2.62 mmol, CAS #173405-78-2) in dioxane (15 mL) was added Pd-PEPPSI-IHEPTCl 3-Chloropyridine (212 mg, 218 umol) and Cs₂CO₃ (1.42 g, 4.36 mmol). Then the mixture was stirred at 100° C. for 10 hours. On completion, the mixture was filtered and concentrated in vacuo to give the residue. The residue was purified by reverse-phase (0.1% FA) to give the title compound (400 mg, 29% yield) as yellow solid. ¹H NMR (400 MHz, CDCl₃-d) δ 7.40-7.35 (m, 2H), 6.93-6.86 (m, 2H), 6.86-6.80 (m, 2H), 6.28 (d, J=7.2 Hz, 1H), 5.21 (dd, J=5.4, 13.0 Hz, 1H), 5.02-4.92 (m, 2H), 3.80 (s, 3H), 3.76 (s, 3H), 3.47-3.39 (m, 4H), 3.04-2.90 (m, 5H), 2.87-2.77 (m, 1H), 2.68-2.55 (m, 1H), 2.18-2.11 (m, 1H), 1.74 (s, 2H), 1.65-1.63 (m, 4H), 1.48 (s, 9H), 1.43 (s, 2H). LC-MS (ESI⁺) m/z 632.4 (M+H)⁺.

### Step 2—3-[4-(3,9-Diazaspiro[5.5]undecan-3-yl)-3-methyl-2-oxo-benzimidazol-1-yl]piperidine-2,6-dione

To a solution of tert-butyl 9-[1-[1-[(4-methoxyphenyl)methyl]-2,6-dioxo-3-piperidyl]-3-methyl-2-oxo-benzimidazol-4-yl]-3,9-diazaspiro[5.5]undecane-3-carboxylate (200 mg, 316 umol) in TFA (2 mL) was added TfOH (0.2 mL). Then the mixture was stirred at 60° C. for 2 hours. On completion, the mixture was concentrated in vacuo to give the title compound (150 mg, 90% yield, TFA) as a brown solid. LC-MS (ESI⁺) m/z 412.2 (M+H)⁺.

### Step 3—Tert-butyl 9-[1-(2,6-dioxo-3-piperidyl)-3-methyl-2-oxo-benzimidazol-4-yl]-3,9-diazaspiro [5.5]undecane-3-carboxylate

To a solution of 3-[4-(3,9-diazaspiro[5.5]undecan-3-yl)-3-methyl-2-oxo-benzimidazol-1-yl]piperidine-2,6-dione (150 mg, 285 umol, TFA) in DCM (4 mL) was added TEA (79.4 uL, 570 umol) and Boc₂O (93.4 mg, 428 umol), then the mixture was stirred at 25° C. for 10 hours. On completion, the mixture was diluted with DCM (100 mL) and extracted with water (50 mL×3). The combined organic layer was dried over anhydrous Na₂SO₄, filtered and concentrated in vacuo to give the title compound (140 mg, 96% yield) as a brown solid. ¹H NMR (400 MHz, CDCl₃-d) δ 8.46-8.34 (m, 1H), 7.02-6.92 (m, 2H), 6.57 (d, J=7.2 Hz, 1H), 5.22 (dd, J=5.2, 12.4 Hz, 1H), 3.99-3.90 (m, 1H), 3.77 (s, 3H), 3.46-3.40 (m, 4H), 2.97-2.88 (m, 5H), 2.83 (dd, J=4.8, 13.2 Hz, 1H), 2.77-2.73 (m, J=4.0, 15.2 Hz, 1H), 2.22-2.16 (m, J=2.8, 5.2, 10.8 Hz, 1H), 1.74 (s, 3H), 1.65-1.63 (m, J=8.0 Hz, 4H), 1.48 (s, 9H). LC-MS (ESI⁺) m/z 512.4 (M+H)⁺.

### Step 4—3-[4-(3,9-Diazaspiro[5.5]undecan-3-yl)-3-methyl-2-oxo-benzimidazol-1-yl]piperidine-2,6-dione

To a solution of tert-butyl 9-[1-(2,6-dioxo-3-piperidyl)-3-methyl-2-oxo-benzimidazol-4-yl]-3,9-diazaspiro[5.5]undecane-3-carboxylate (80.0 mg, 156 umol) in DCM (2 mL) was added TFA (1 mL, 13.5 mmol), then the mixture was stirred at 25° C. for 1 hour. On completion, the mixture was concentrated in vacuo to give the title compound (80.0 mg, 97% yield, TFA) as a blackibrown solid. LC-MS (ESI⁺) m/z 412.2 (M+H)⁺.

### 5-cyano-N-[2-(4-formylcyclohexyl)-6-[(1R,4R)-2-oxa-5-azabicyclo[2.2.1]heptan-5-yl]indazol-5-yl]pyridine-3-carboxamide (Intermediate BXS)

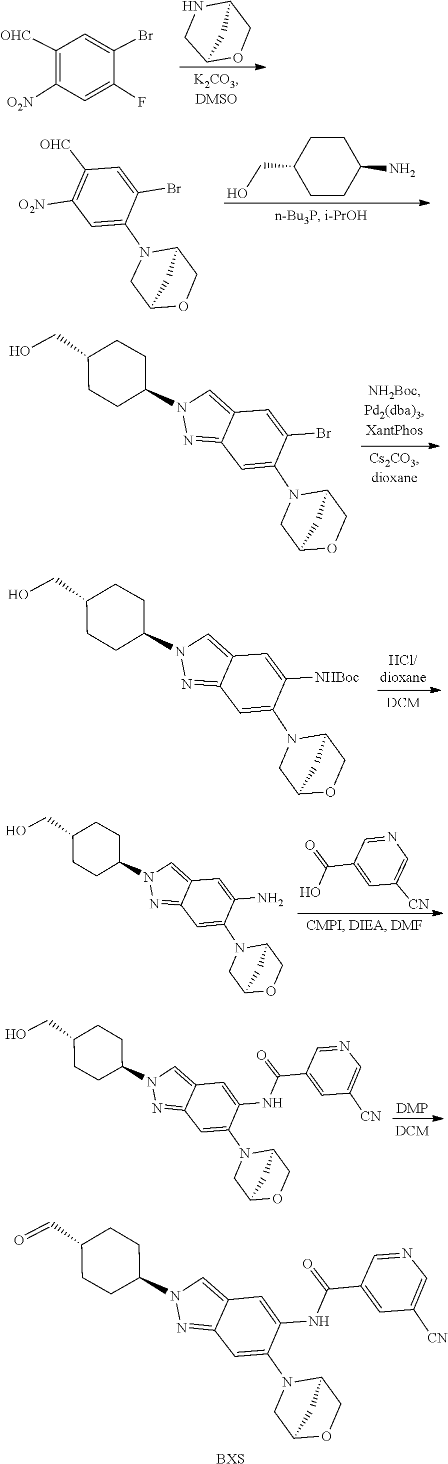

### Step 1—5-bromo-2-nitro-4-[(1R,4R)-2-oxa-5-azabicyclo[2.2.1]heptan-5-yl]benzaldehyde

To a solution of (1R,4R)-2-oxa-5-azabicyclo[2.2.1]heptane (1.23 g, 9.07 mmol, HCl salt, CAS #66147-56-0) and K₂CO₃ (1.67 g, 12.10 mmol) in DMSO (15 mL) was added 5-bromo-4-fluoro-2-nitro-benzaldehyde (1.5 g, 6.05 mmol, CAS #213382-45-7). Then the reaction mixture was stirred at 80° C. for 2 hours. On completion, the reaction was diluted with EtOAc (100 mL) and washed with brine (40 mL×4). The organic layer was dried over Na₂SO₄ and filtered. The filtrate was concentrated in vacuo to give the title compound (1.50 g, 76% yield) as yellow solid. ¹H NMR (400 MHz, CDCl₃) δ 10.22 (s, 1H), 8.17 (s, 1H), 7.21 (s, 1H), 4.83 (d, J=1.2 Hz, 1H), 4.72 (s, 1H), 4.12-4.07 (m, 2H), 3.99 (dd, J=1.6, 8.0 Hz, 1H), 3.48 (d, J=10.0 Hz, 1H), 2.10-2.05 (m, 2H).

### Step 2—[4-[5-bromo-6-[(1R,4R)-2-oxa-5-azabicyclo[2.2.1]heptan-5-yl]indazol-2-yl]cyclohexyl]methanol

To a solution of 5-bromo-2-nitro-4-[(1R,4R)-2-oxa-5-azabicyclo[2.2.1]heptan-5-yl]benzaldehyde (1.50 g, 4.59 mmol) in i-PrOH (15 mL) was added (4-aminocyclohexyl)methanol (710 mg, 5.50 mmol, CAS #1467-84-1). The mixture was heated at 80° C. for 8 hours under N₂. Then, the mixture was cooled to 25° C. and tritert-butylphosphane (2.78 g, 13.7 mmol) was added. The reaction mixture was then heated to 80° C. for 10 hours. On completion, the reaction mixture was concentrated in vacuo. The residue was purified by column chromatography (SiO₂, PE:EA=3:1 to 0:1, PE:EA=0:1, P1:Rf=0.3) to give the title compound (1 g, 54% yield) as yellow solid. ¹H NMR (400 MHz, DMSO-d₆) δ 8.23 (s, 1H), 7.94 (s, 1H), 7.13 (s, 1H), 4.56 (s, 1H), 4.48 (t, J=5.2 Hz, 1H), 4.41-4.33 (m, 1H), 4.33-4.29 (m, 1H), 3.96 (d, J=7.6 Hz, 1H), 3.73 (dd, J=1.6, 7.5 Hz, 1H), 3.52 (dd, J=1.2, 9.8 Hz, 1H), 3.28 (t, J=5.6 Hz, 2H), 3.24 (d, J=10.1 Hz, 1H), 2.14-2.05 (m, 2H), 1.91-1.76 (m, 6H), 1.63-1.52 (m, 1H), 1.19-1.10 (m, 2H).

### Step 3—Tert-butyl N-[2-[4-(hydroxymethyl)cyclohexyl]-6-[(1R,4R)-2-oxa-5-azabicyclo[2.2.1]heptan-5-yl]indazol-5-yl]carbamate

To a solution of [4-[5-bromo-6-[(1R,4R)-2-oxa-5-azabicyclo[2.2.1]heptan-5-yl]indazol-2-yl]cyclohexyl]methanol (500 mg, 1.23 mmol), NH₂Boc (288 mg, 2.46 mmol), Xantphos (142 mg, 246 umol) and Cs₂CO₃ (1.20 g, 3.69 mmol) in dioxane (5 mL) was added Pd₂(dba)₃ (112 mg, 123 umol). Then the mixture was stirred at 110° C. for 16 hours under N₂. On completion, the reaction mixture was filtered and the filtrate was concentrated in vacuo. The residue was purified by prep-HPLC (column: YMC Triart C18 250*50 mm*7 um; mobile phase: [water (0.225% FA)-ACN]; B %: 35%-65%, min) to give the title compound (140 mg, 26% yield) as yellow solid. ¹H NMR (400 MHz, DMSO-d₆) δ 8.20-8.13 (m, 1H), 8.08 (s, 1H), 7.48 (s, 1H), 6.86 (s, 1H), 4.54 (s, 1H), 4.39-4.27 (m, 1H), 4.21 (s, 1H), 3.97 (d, J=7.6 Hz, 1H), 3.72 (d, J=7.2 Hz, 1H), 3.46 (d, J=9.6 Hz, 1H), 3.28 (d, J=6.0 Hz, 2H), 3.04 (d, J=9.6 Hz, 1H), 2.12-2.07 (m, 3H), 1.95-1.75 (m, 6H), 1.61-1.47 (m, 1H), 1.43 (s, 9H), 1.28-1.05 (m, 2H).

### Step 4—[4-[5-amino-6-[(1R,4R)-2-oxa-5-azabicyclo[2.2.1]heptan-5-yl]indazol-2-yl]cyclohexyl] methanol

To a solution of tert-butyl N-[2-[4-(hydroxymethyl)cyclohexyl]-6-[(1R,4R)-2-oxa-5-azabicyclo[2.2.1]heptan-5-yl]indazol-5-yl]carbamate (140 mg, 316 umol) in DCM (1 mL) was added HCl/dioxane (4 M, 12.6 mL), then the reaction mixture was stirred at 25° C. for 2 hours. On completion, the reaction mixture was concentrated in vacuo to give the title compound (119 mg, 99% yield, HCl) as brown solid. LC-MS (ESI⁺) m/z 343.1 (M+H)⁺.

### Step 5—5-cyano-N-[2-[4-(hydroxymethyl)cyclohexyl]-6-[(1R,4R)-2-oxa-5-azabicyclo[2.2.1]heptan-5-yl]indazol-5-yl]pyridine-3-carboxamide

To a mixture of 5-cyanopyridine-3-carboxylic acid (46.5 mg, 314 umol, CAS #887575-62-6) and DIEA (60.8 mg, 471 umol) in DMF (1 mL) was added CMPI (80.2 mg, 314 umol), then the mixture was stirred at 20° C. for 10 mins. Then the mixture was add into a solution of [4-[5-amino-6-[(1R,4R)-2-oxa-5-azabicyclo[2.2.1]heptan-5-yl]indazol-2-yl]cyclohexyl]methanol (119 mg, 314 umol, HCl salt) and DIEA (60.8 mg, 471 umol) in DMF (0.5 mL) at 20° C. The reaction mixture was stirred at 20° C. for 2 hours. On completion, the reaction mixture was added dropwise into H₂O (100 mL), filtered and the filtrate was concentrated in vacuo. The residue was purified by prep-HPLC (column: Phenomenex Synergi C18 100*21.2 mm*4 um; mobile phase: [water (0.225% FA)-ACN]; B %: 17%-47%, 11.5 min) to give the title compound (90 mg, 61% yield) as brown solid. ¹H NMR (400 MHz, DMSO-d₆) δ 10.10 (s, 1H), 9.36 (d, J=2.0 Hz, 1H), 9.21 (d, J=1.6 Hz, 1H), 8.80 (s, 1H), 8.25 (s, 1H), 7.64 (s, 1H), 6.94 (s, 1H), 4.47 (s, 2H), 4.40-4.29 (m, 2H), 4.01 (d, J=7.6 Hz, 1H), 3.72 (d, J=6.4 Hz, 1H), 3.43 (d, J=8.4 Hz, 1H), 3.29 (s, 2H), 2.99 (d, J=9.6 Hz, 1H), 2.13 (d, J=9.2 Hz, 2H), 1.93-1.81 (m, 5H), 1.76 (d, J=9.6 Hz, 1H), 1.53-1.41 (m, 1H), 1.20-1.07 (m, 2H).

### Step 6—5-cyano-N-[2-(4-formylcyclohexyl)-6-[(1R,4R)-2-oxa-5-azabicyclo[2.2.1]heptan-5-yl] indazol-5-yl]pyridine-3-carboxamide

To a solution of 5-cyano-N-[2-[4-(hydroxymethyl)cyclohexyl]-6-[(1R,4R)-2-oxa-5-azabicyclo[2.2.1]heptan-5-yl]indazol-5-yl]pyridine-3-carboxamide (80.0 mg, 169 umol) in DCM (1 mL) was added DMP (93.3 mg, 220 umol), then the reaction mixture was stirred at 25° C. for 2 hours. On completion, the reaction mixture was quenched with saturated Na₂S₂O₃ (15 mL) and saturated NaHCO₃ (10 mL) at 25° C., and then stirred for 15 minutes. The mixture was extracted with DCM (30 mL×2), then the combined organic layers were washed with saturated NaCl (20 mL×2) and dried over Na₂SO₄, and filtered. The filtrate was concentrated in vacuo to give the title compound (80 mg, 40% yield) as white solid. LC-MS (ESI⁺) m/z 471.2 (M+H)⁺.

### 2-fluoro-3-(trifluoromethyl)benzoic acid (CAS #115029-22-6) (Intermediate BXV)

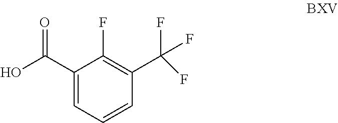 [SMILES: O=C(O)c1cccc(C(F)(F)F)c1F]

### 4-(trifluoromethyl)thiazole-2-carboxylic acid (CAS #944900-55-4) (Intermediate BXW)

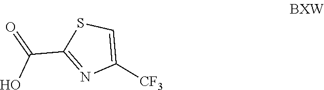 [SMILES: O=C(O)c1nc(C(F)(F)F)cs1]

### 2-Fluoro-N-[2-(4-formylcyclohexyl)-6-methoxy-indazol-5-yl]-3-(trifluoromethyl)benzamide (Intermediate BXX)

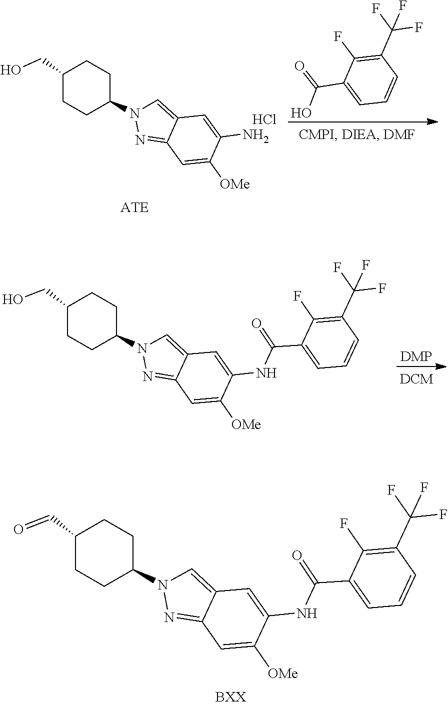

### Step 1—2-Fluoro-N-[2-[4-(hydroxymethyl)cyclohexyl]-6-methoxy-indazol-5-yl]-3-(trifluoromethyl) benzamide

To a solution of [4-(5-amino-6-methoxy-indazol-2-yl)cyclohexyl]methanol (300 mg, 962 umol, HCl, Intermediate ATE) and 2-fluoro-3-(trifluoromethyl)benzoic acid (200 mg, 962 umol, CAS #115029-22-6) in DMF (5 mL) was added DIEA (335 uL, 1.92 mmol) and CMPI (295 mg, 1.15 mmol), then the mixture was stirred at 25° C. for 1 hour. On completion, the mixture was poured into water (15 mL), then filtered and the filter cake was dried in vacuo to give the title compound (311 mg, 69% yield) as a brown solid. LC-MS (ESI⁺) m/z 466.2 (M+H)⁺.

### Step 2—2-Fluoro-N-[2-(4-formlylcyclohexyl)-6-methoxy-indazol-5-yl]-3-(trifluoromethyl)benzamide

To a solution of 2-fluoro-N-[2-[4-(hydroxymethyl)cyclohexyl]-6-methoxy-indazol-5-yl]-3-(trifluoromethyl)benzamide (150 mg, 322 umol) in DCM (3 mL) was added DMP (150 uL, 483 umol), then the mixture was stirred at 25° C. for 1 hour. On completion, the mixture was quenched with sodium thiosulfate pentahydrate (3 mL) and NaHCO₃ (3 mL), then extracted with DCM (20 mL×3). The combined organic layer was concentrated in vacuo to give the title compound (140 mg, 93% yield) as a brown solid. ¹H NMR (400 MHz, DMSO-d₆) δ 9.74 (d, J=4.0 Hz, 1H), 9.64 (s, 1H), 8.36 (s, 1H), 8.31 (s, 1H), 8.08 (t, J=6.8 Hz, 1H), 7.55 (t, J=7.6 Hz, 1H), 7.09 (s, 1H), 4.40 (tt, J=3.6, 11.6 Hz, 1H), 3.90 (s, 3H), 2.45-2.37 (m, 1H), 2.20 (dd, J=2.8, 12.4 Hz, 2H), 2.14-2.07 (m, 2H), 1.96 (dt, J=9.2, 12.4 Hz, 2H), 1.44 (dq, J=3.2, 12.8 Hz, 2H), 1.23 (s, 1H). LC-MS (ESI⁺) m/z 464.2 (M+H)⁺.

### 1-[8-(3,8-Diazabicyclo[3.2.1]octan-3-yl)-4-isoquinolyl]hexahydropyrimidine-2,4-dione (Intermediate BXY)

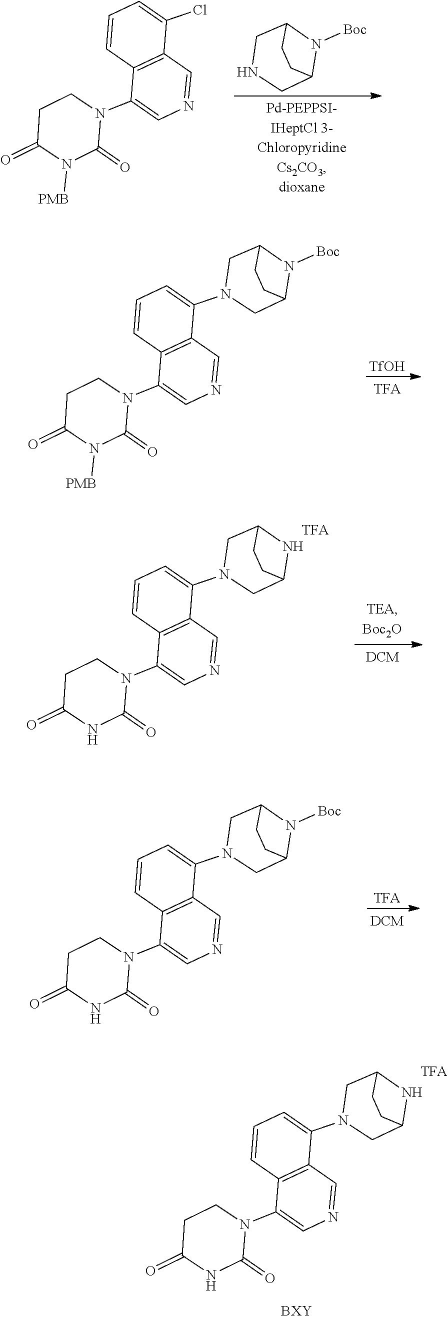

### Step 1—Tert-butyl 3-[4-[3-[(4-methoxyphenyl)methyl]-2,4-dioxo-hexahydropyrimidin-1-yl]-8-isoquinolyl]-3,8-diazabicyclo[3.2.1]octane-8-carboxylate

To a solution of 1-(8-chloro-4-isoquinolyl)-3-[(4-methoxyphenyl)methyl]hexahydropyrimidine-2,4-dione (500 mg, 1.26 mmol, synthesized via Steps 1-2 of Intermediat BSL) and tert-butyl3,8-diazabicyclo[3.2.1]octane-8-carboxylate (321 mg, 1.52 mmol, CAS #149771-44-8) in dioxane (8 mL) was added Pd-PEPPSI-IHEPTCl ₃-Chloropyridine (123 mg, 126 umol) and Cs₂CO₃ (823 mg, 2.53 mmol). Then the mixture was stirred at 80° C. for 8 hours. On completion, the mixture was filtered and the filtrate was extracted with water (50 mL) and EA (30 mL×3). The combined organic layer was concentrated in vacuo to give the title compound (660 mg, 91% yield) as a yellow solid. ¹H NMR (400 MHz, CDCl₃-d) δ 9.66 (s, 1H), 8.45 (s, 1H), 7.61 (t, J=8.0 Hz, 1H), 7.41 (d, J=8.4 Hz, 2H), 7.32 (d, J=8.4 Hz, 1H), 7.20 (d, J=7.6 Hz, 1H), 6.83 (d, J=8.8 Hz, 2H), 4.98 (s, 2H), 4.46-4.30 (m, 2H), 3.89-3.80 (m, 1H), 3.78 (s, 3H), 3.73-3.67 (m, 1H), 3.27-3.07 (m, 4H), 3.02-2.96 (m, 2H), 2.30-2.17 (m, 2H), 1.50 (s, 9H), 1.47 (d, J=2.0 Hz, 2H). LC-MS (ESI⁺) m/z 572.2 (M+H)⁺.

### Step 2—1-[8-(3,8-Diazabicyclo[3.2.1]octan-3-yl)-4-isoquinolyl]hexahydropyrimidine-2,4-dione

To a solution of tert-butyl 3-[4-[3-[(4-methoxyphenyl)methyl]-2,4-dioxo-hexahydropyrimidin-1-yl]-8-isoquinolyl]-3,8-diazabicyclo[3.2.1]octane-8-carboxylate (660 mg, 1.15 mmol) in TFA (6 mL) was added TfOH (1 mL), then the mixture was stirred at 70° C. for 2 hours. On completion, the mixture was concentrated in vacuo to give the title compound (500 mg, 93% yield, TFA) as a brown solid.

### Step 3—Tert-butyl 3-[4-(2,4-dioxohexahydropyrimidin-1-yl)-8-isoquinolyl]-3,8-diazabicyclo[3.2.1]octane-8-carboxylate

To a solution of 1-[8-(3,8-diazabicyclo[3.2.1]octan-3-yl)-4-isoquinolyl]hexahydropyrimidine-2,4-dione (500 mg, 1.07 mmol, TFA) in DCM (5 mL) was added TEA (299 uL, 2.15 mmol) and Boc₂O (370 uL, 1.61 mmol), then the mixture was stirred at 25° C. for 10 hours. On completion, the mixture was filtered and filtrate was extracted with DCM (50 mL) and water (30 mL×3). The combined organic layer was dried over anhydrous Na₂SO₄, then concentrated in vacuo to give the residue. The residue was purified by reverse-phase (0.1% FA condition) to give the title compound (212 mg, 44% yield) as a yellow solid. ¹H NMR (400 MHz, CDCl₃-d) δ 9.73 (s, 1H), 8.56 (s, 1H), 7.85 (s, 1H), 7.77-7.71 (m, 1H), 7.50 (d, J=8.4 Hz, 1H), 7.29 (s, 1H), 4.06-3.95 (m, 1H), 3.86 (td, J=6.0, 12.4 Hz, 1H), 3.79-3.70 (m, 1H), 3.33-3.10 (m, 4H), 3.06-2.95 (m, 2H), 2.28 (d, J=8.4 Hz, 2H), 2.17-2.03 (m, 3H), 1.53 (s, 9H). LC-MS (ESI⁺) m/z 452.2 (M+H)⁺.

### Step 4—1-[8-(3,8-Diazabicyclo[3.2.1]octan-3-yl)-4-isoquinolyl]hexahydropyrimidine-2,4-dione

To a solution of tert-butyl 3-[4-(2,4-dioxohexahydropyrimidin-1-yl)-8-isoquinolyl]-3,8-diazabicyclo [3.2.1]octane-8-carboxylate (70.0 mg, 155 umol) in DCM (1.5 mL) was added TFA (0.5 mL, 6.75 mmol), then the mixture was stirred at 25° C. for 1 hour. On completion, the mixture was concentrated in vacuo to give the title compound (60.0 mg, 83% yield, TFA) as a brown solid. LC-MS (ESI⁺) m/z 352.2 (M+H)⁺.

### 1-[8-(3,6-Diazabicyclo[3.1.1]heptan-3-yl)-4-isoquinolyl]hexahydropyrimidine-2,4-dione (Intermediate BXZ)

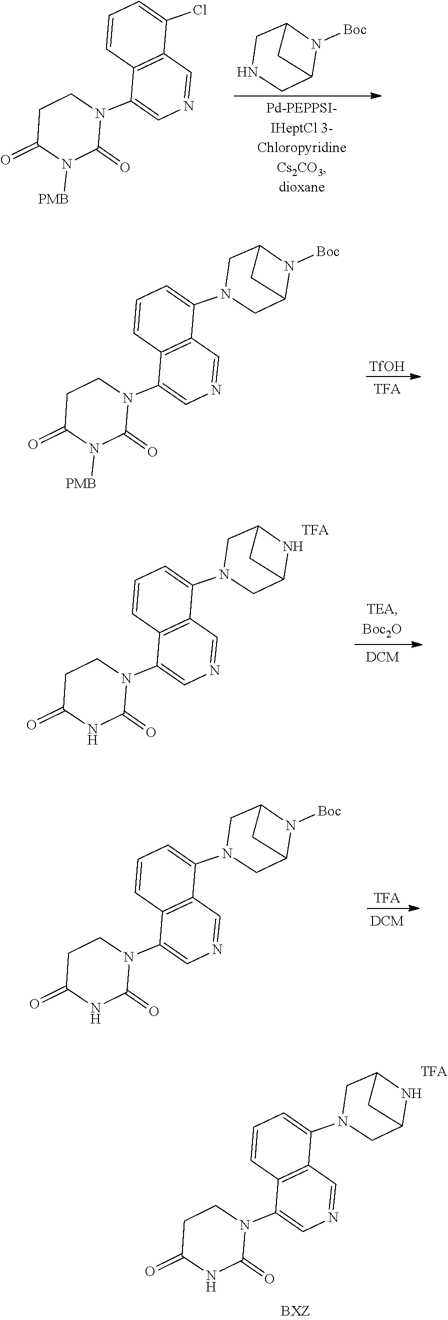

### Step 1—Tert-butyl 3-[4-[3-[(4-methoxyphenyl)methyl]-2,4-dioxo-hexahydropyrimidin-1-yl]-8-isoquinolyl]-3,6-diazabicyclo[3.1.1]heptane-6-carboxylate

To a solution of 1-(8-chloro-4-isoquinolyl)-3-[(4-methoxyphenyl)methyl]hexahydropyrimidine-2,4-dione (500 mg, 1.26 mmol, synthesized via Steps 1-2 of Intermediate BSL) and tert-butyl3,6-diazabicyclo[3.1.1]heptane-6-carboxylate (275 mg, 1.39 mmol, CAS #869494-16-6) in dioxane (10 mL) was added Pd-PEPPSI-IHEPTCl 3-Chloropyridine (122 mg, 126 umol) and Cs₂CO₃ (823 mg, 2.53 mmol), then the mixture was stirred at 80° C. for 10 hours. On completion, the mixture was filtered and diluted with water (40 mL), then extracted with EA (4×50 mL), dried over anhydrous Na₂SO₄, filtered and concentrated in vacuo to give the title compound (650 mg, 92% yield) as a yellow solid. ¹H NMR (400 MHz, DMSO-d₆) δ 9.58 (s, 1H), 8.51 (s, 1H), 7.75-7.68 (m, 1H), 7.56 (d, J=8.4 Hz, 1H), 7.47 (d, J=7.6 Hz, 1H), 7.26 (d, J=8.4 Hz, 2H), 6.88 (d, J=8.4 Hz, 2H), 4.83 (s, 2H), 4.20 (s, 2H), 3.73 (s, 3H), 3.33 (s, 4H), 3.17-3.06 (m, 1H), 3.00-2.91 (m, 1H), 2.61-2.53 (m, 1H), 1.66 (d, J=7.6 Hz, 1H), 1.37 (s, 9H), 0.88-0.72 (m, 2H). LC-MS (ESI⁺) m/z 558.3 (M+H)⁺.

### Step 2—1-[8-(3,6-Diazabicyclo[3.1.1]heptan-3-yl)-4-isoquinolyl]hexahydropyrimidine-2,4-dione

To a solution of tert-butyl 3-[4-[3-[(4-methoxyphenyl)methyl]-2,4-dioxo-hexahydropyrimidin-1-yl]-8-isoquinolyl]-3,6-diazabicyclo[3.1.1]heptane-6-carboxylate (600 mg, 1.08 mmol) in TFA (6 mL) was added TfOH (1.70 g, 11.3 mmol), then the mixture was stirred at 70° C. for 2 hours. On completion, the mixture was concentrated in vacuo to give the title compound (330 mg, 90% yield) as a yellow oil.

### Step 3—Tert-butyl 3-[4-(2,4-dioxohexahydropyrimidin-1-yl)-8-isoquinolyl]-3,6-diazabicyclo[3.1.1]heptane-6-carboxylate

To a solution of 1-[8-(3,6-diazabicyclo[3.1.1]heptan-3-yl)-4-isoquinolyl]hexahydropyrimidine-2,4-dione (330 mg, 978 umol) in DCM (5 mL) was added TEA (198 mg, 1.96 mmol) and Boc₂O (320 mg, 1.47 mmol), then the mixture was stirred at 25° C. for 8 hours. On completion, the mixture was diluted with DCM (30 mL) and water (50 mL), then extracted with DCM (4×70 mL), dried over anhydrous Na₂SO₄, filtered and concentrated in vacuo to give the residue. The residue was purified by reverse-phase (0.10% FA) to give the title compound (200 mg, 46% yield) as a yellow solid. LC-MS (ESI⁺) m/z 438.2 (M+H)⁺.

### Step 4—1-[8-(3,6-Diazabicyclo[3.1.1]heptan-3-yl)-4-isoquinolyl]hexahydropyrimidine-2,4-dione

To a solution of tert-butyl 3-[4-(2,4-dioxohexahydropyrimidin-1-yl)-8-isoquinolyl]-3,6-diazabicyclo [3.1.1]heptane-6-carboxylate (70.0 mg, 160 umol) in DCM (1.5 mL) was added TFA (770 mg, 6.75 mmol), then the mixture was stirred at 25° C. for 1 hour. On completion, the mixture was concentrated in vacuo to give the title compound (70.0 mg, 96% yield, TFA) as a yellow oil. LC-MS (ESI⁺) m/z 338.1 (M+H)⁺.

### (1R,3R)-methyl 3-(5-methoxy-6-(6-(trifluoromethyl)picolinamido)benzo[d]thiazol-2-yl) cyclobutanecarboxylate (Intermediate CAA)

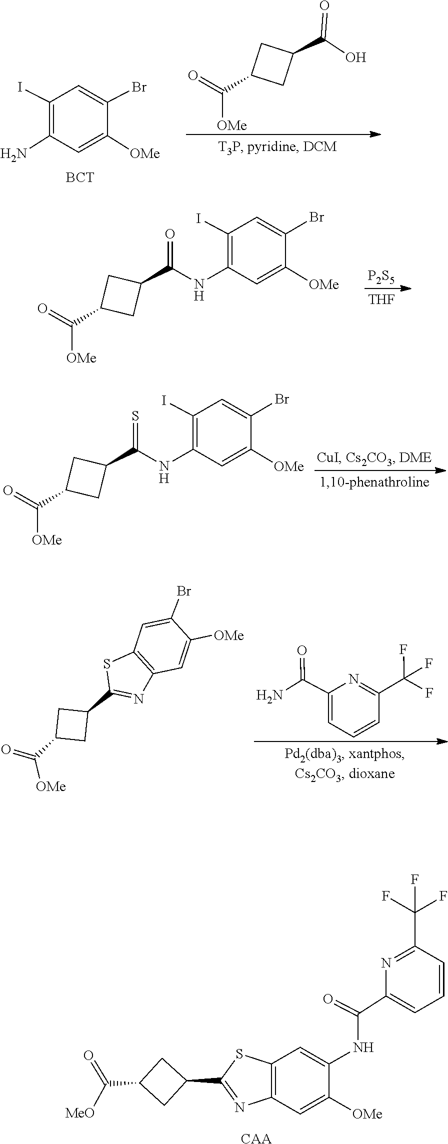

### Step 1—(1r, 3r)-Methyl 3-((4-bromo-2-iodo-5-methoxyphenyl)carbamoyl)cyclobutanecarboxylate

To a solution of 3-methoxycarbonylcyclobutanecarboxylic acid (2.00 g, 12.6 mmol, CA S #1401103-71-6), 4-bromo-2-iodo-5-methoxy-aniline (4.15 g, 12.6 mmol, Intermediate BCT) in DCM (50 mL) was added T₃P (12.0 g, 18.9 mmol) and pyridine (5.00 g, 63.2 mmol). The mixture was stirred at 25° C. for 3 hours. On completion, the reaction mixture was diluted with water (50 mL), then extracted with EA (3×50 ml). The combined organic layers were concentrated in vacuo to give the title compound (5.9 g, 99% yield) as a white solid. LC-MS (ESI⁺) m/z 467.8 (M+H)⁺.

### Step 2—(1R,3R)-methyl 3-((4-bromo-2-iodo-5-methoxyphenyl)carbamothioyl)cyclobutane carboxylate

To a solution of methyl 3-[(4-bromo-2-iodo-5-methoxy-phenyl)carbamoyl]-cyclobutanecarboxylate (5.90 g, 12.6 mmol) in THF (60 mL) was added P₂S₅(3.36 g, 15.1 mmol). The reaction mixture was stirred at 25° C. for 5 hours. On completion, the reaction was filtered, and the filtrate was concentrated in vacuo to give the title compound (6.1 g, 99% yield) as a white solid.

### Step 3—(1R,3R)-methyl 3-(6-bromo-5-methoxybenzo[d]thiazol-2-yl)cyclobutanecarboxylate

A mixture of methyl 3-[(4-bromo-2-iodo-5-methoxy-phenyl)carbamothioyl]cyclobutanecarboxylate (6.10 g, 12.6 mmol), CuI (239 mg, 1.26 mmol), 1,10-phenanthroline (227 mg, 1.26 mmol) and Cs₂CO₃ (8.21 g, 25.2 mmol) in DME (100 mL) was degassed and purged with N₂ three times. Then the mixture was stirred at 40° C. for 3 hours under N₂ atmosphere. On completion, the reaction was filtered, and the filtrate was concentrated in vacuo. The residue was purified by column chromatography (SiO₂, Petroleum ether/Ethyl acetate=10/1 to 5/1) to give the title compound (2.4 g, 53% yield) as a white solid. LC-MS (ESI⁺) m/z 358.0 (M+H)⁺.

### Step 4—(1R,3R)-methyl 3-(5-methoxy-6-(6-(trifluoromethyl)picolinamido)benzo[d]thiazol-2-yl) cyclobutanecarboxylate

A mixture of methyl 3-(6-bromo-5-methoxy-1,3-benzothiazol-2-yl)cyclobutanecarboxylate (1.20 g, 3.37 mmol), 6-(trifluoromethyl)pyridine-2-carboxamide (768 mg, 4.04 mmol, CAS-22245-84-7), Pd₂(dba)₃ (308 mg, 336 umol,), Xantphos (389 mg, 673 umol) and Cs₂CO₃ (3.29 g, 10.1 mmol) in dioxane (10.0 mL) was degassed and purged with N₂ three times. Then the mixture was stirred at 80° C. for 16 hours under N₂ atmosphere. On completion, the reaction was filtered, and the filtrate was concentrated in vacuo. The residue was purified by column chromatography (SiO₂, Petroleum ether/Ethyl acetate=10/1 to 3/1) to give the title compound (1.2 g, 71% yield) as a white solid. ¹H NMR (400 MHz, CDCl₃) δ 10.63 (s, 1H), 9.04 (s, 1H), 8.43 (d, J=8.0 Hz, 1H), 8.07 (t, J=8.0 Hz, 1H), 7.81 (dd, J=0.8, 8.0 Hz, 1H), 7.48 (s, 1H), 7.46 (s, 1H), 4.01-3.95 (m, 4H), 3.69 (s, 3H), 3.65 (s, 1H), 3.34-3.23 (m, 1H), 2.81-2.65 (m, 5H); LC-MS (ESI⁺) m/z 466.0 (M+H)⁺.

### 1-[8-[(3R)-3-methylpiperazin-1-yl]-4-isoquinolyl]hexahydropyrimidine-2,4-dione (Intermediate CAB)

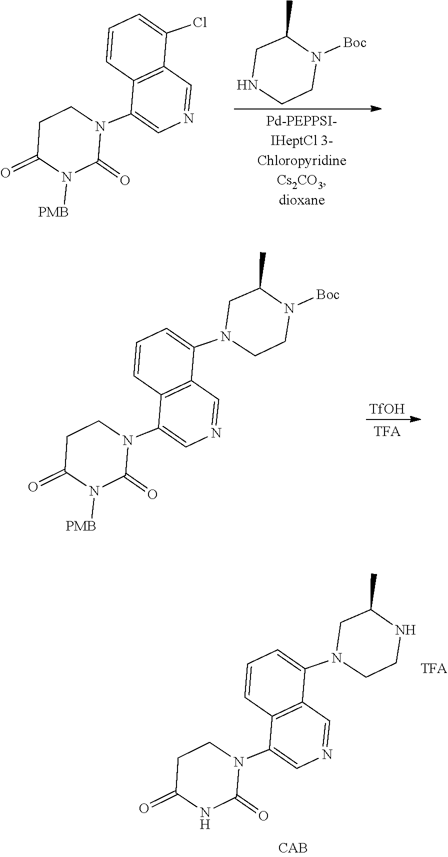

### Step 1—Tert-butyl (2R)-4-[4-[3-[(4-methoxyphenyl)methyl]-2,4-dioxo-hexahydropyrimidin-1-yl]-8-isoquinolyl]-2-methyl-piperazine-1-carboxylate

A mixture of 1-(8-chloro-4-isoquinolyl)-3-[(4-methoxyphenyl)methyl]hexahydropyrimidine-2,4-dione (200 mg, 505 umol, synthesized via Steps 1-2 of Intermediate BSL), tert-butyl (2R)-2-methylpiperazine-1-carboxylate (111 mg, 555 umol, CAS #170033-47-3), Cs₂CO₃ (493 mg, 1.52 mmol), 4 Å molecular sieves (100 mg, 505 umol) and PD-PEPPSI-IHeptCl 3-Chloropyridine (100 mg, 252 umol) was dissolved in dioxane (6 mL). The reaction mixture was then stirred at 100° C. for 12 hrs. On completion, the residue was diluted with water (30 mL), then the residue was extracted with EA (3×70 mL). The combined organic layer was dried over Na₂SO₄, filtered and concentrated in vacuo to give a residue. The residue was purified by reverse phase (0.1% FA condition) to give title compound (250 mg, 88% yield) as white solid. ¹H NMR (400 MHz, DMSO-d₆) δ 9.54 (s, 1H), 8.56 (m, 1H), 7.74-7.69 (m, 1H), 7.59 (d, J=8.4 Hz, 1H), 7.30-7.24 (m, 3H), 6.90-6.86 (m, 2H), 4.83 (s, 2H), 4.33-4.29 (m, 1H), 3.96-3.87 (m, 2H), 3.76-3.74 (m, 1H), 3.73 (s, 3H), 3.46-3.43 (m, 1H), 3.20-3.08 (m, 2H), 3.00-2.92 (m, 2H), 2.82-2.73 (m, 3H), 1.44 (s, 9H). LC-MS (ESI⁺) m/z 560.3 (M+H)⁺.

### Step 2—1-[8-[(3R)-3-methylpiperazin-1-yl]-4-isoquinolyl]hexahydropyrimidine-2,4-dione

To a solution of tert-butyl (2R)-4-[4-[3-[(4-methoxyphenyl)methyl]-2,4-dioxo-hexahydropyrimidin-1-yl]-8-isoquinolyl]-2-methyl-piperazine-1-carboxylate (100 mg, 178 umol) in TFA (1 mL) was added TfOH (0.2 mL). The reaction mixture was stirred at 70° C. for 0.2 hr. On completion, the reaction mixture was concentrated in vacuo to give title compound (80.0 mg, 98% yield, TFA) as brown oil. LC-MS (ESI⁺) m/z 339.1 (M+H)⁺.

### 1-[8-[(2S)-2-methylpiperazin-1-yl]-4-isoquinolyl]hexahydropyrimidine-2,4-dione (Intermediate CAC)

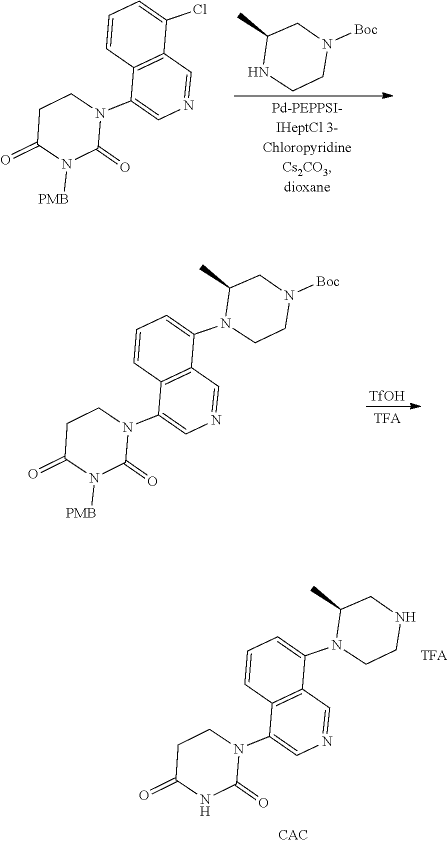

### Step 1—Tert-butyl (3S)-4-[4-[3-[(4-methoxyphenyl)methyl]-2,4-dioxo-hexahydropyrimidin-1-yl]-8-isoquinolyl]-3-methyl-piperazine-1-carboxylate

A mixture of 1-(8-chloro-4-isoquinolyl)-3-[(4-methoxyphenyl)methyl]hexahydropyrimidine-2,4-dione (170 mg, 429 umol, synthesized via Steps 1-2 of Intermediate BSL), tert-butyl (3S)-3-methylpiperazine-1-carboxylate (103 mg, 515 umol, from CAS #147081-29-6), Cs₂CO₃ (279 mg, 858 umol), 4 Å molecular sieves (429 umol) and Pd-PEPPSI-IHeptCl 3-Chloropyridine (41.7 mg, 42.9 umol) in dioxane (5 mL) was stirred at 100° C. for 12 hrs under N₂ atmosphere. On completion, the mixture was filtered and concentrated to give a residue. The residue was purified by reversed phase flash (0.10% FA condition) to give the title compound (200 mg, 82% yield) as white solid. ¹H NMR (400 MHz, DMSO-d₆) δ 9.62 (d, J=5.6 Hz, 1H), 8.55 (d, J=2.8 Hz, 1H), 7.79-7.71 (m, 1H), 7.65 (d, J=8.4 Hz, 1H), 7.49-7.40 (m, 1H), 7.26 (d, J=1.6 Hz, 2H), 6.88 (d, J=8.0 Hz, 2H), 4.83 (s, 2H), 4.01-3.82 (m, 2H), 3.80-3.74 (m, 1H), 3.73 (s, 3H), 3.67-3.37 (m, 3H), 3.24-3.06 (m, 3H), 2.96 (d, J=5.6 Hz, 1H), 2.76 (d, J=8.0 Hz, 1H), 1.45 (s, 9H), 0.82 (d, J=6.0 Hz, 3H); LC-MS (ESI+) m/z 560.3 (M+H)⁺.

### Step 2—1-[8-[(2S)-2-methylpiperazin-1-yl]-4-isoquinolyl]hexahydropyrimidine-2,4-dione

To a mixture of tert-butyl (3S)-4-[4-[3-[(4-methoxyphenyl)methyl]-2,4-dioxo-hexahydropyrimidin-1-yl]-8-isoquinolyl]-3-methyl-piperazine-1-carboxylate (100 mg, 178 umol) in TFA (2 mL) was added TfOH (0.4 mL). The mixture was then stirred at 70° C. for 1 hr. On completion, the mixture was concentrated to give the title compound (80.0 mg, 176 umol, TFA) as a brown oil. LC-MS (ESI⁺) m/z 340.1 (M+H)⁺.

### N-(2-((1r, 3r)-3-(iodomethyl)cyclobutyl)-5-methoxybenzo[d]thiazol-6-yl)-6-(trifluoromethyl)picolinamide (Intermediate CAD)

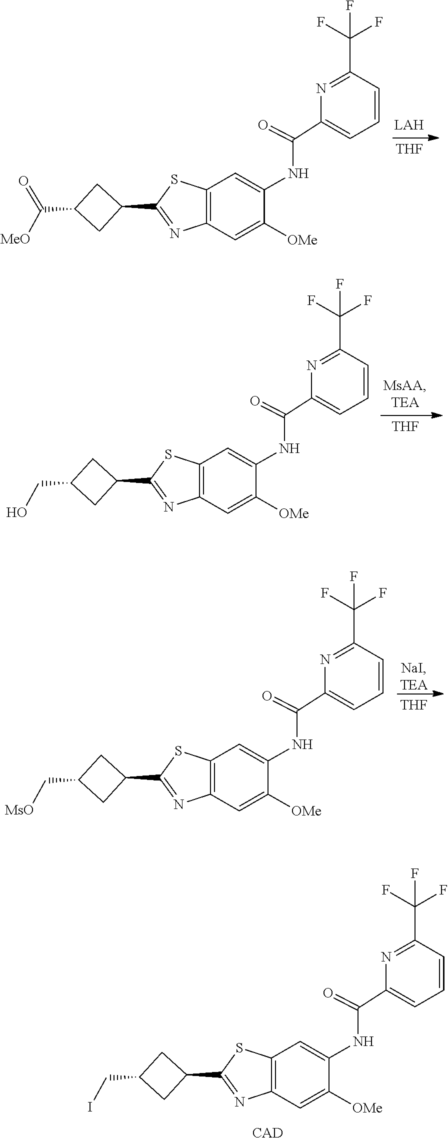

### Step 1—N-(2-((1r, 3r)-3-(hydroxymethyl)cyclobutyl)-5-methoxybenzo[d]thiazol-6-yl)-6-(trifluoromethyl)picolinamide

To a solution of methyl 3-[5-methoxy-6-[[6-(trifluoromethyl)pyridine-2-carbonyl]amino]-1,3-benzothiazol-2-yl]cyclobutanecarboxylate (330 mg, 709 umol, Intermediate CAA) in THF (4 mL) was added LiAlH₄ (40.3 mg, 1.06 mmol). The mixture was then stirred at 0° C. for 2 h. On completion, water (0.05 ml) was added into reaction, then 15% NaOH(aq) (0.05 ml) was added to quench the reaction mixture. The mixture was then filtered and the filtrate was concentrated under vacuum. The residue was purified by column chromatography (SiO₂, Petroleum ether/Ethyl acetate=4/1 to 1.5/1) to give the title compound (120 mg, 39% yield). ¹H NMR (400 MHz, CDCl₃) δ 10.73 (s, 1H), 9.13 (s, 1H), 8.52 (d, J=8.0 Hz, 1H), 8.16 (t, J=7.6 Hz, 1H), 7.90 (dd, J=0.8, 7.6 Hz, 1H), 7.59 (s, 1H), 4.08 (s, 3H), 4.04-3.94 (m, 1H), 3.83 (d, J=6.4 Hz, 2H), 2.78-2.69 (m, 1H), 2.70-2.59 (m, 2H), 2.49-2.38 (m, 2H).

### Step 2—((1r, 3r)-3-(5-methoxy-6-(6-(trifluoromethyl)picolinamido)benzo[d]thiazol-2-yl)cy clobutyl)methyl methanesulfonate

To a solution of N-[2-[3-(hydroxymethyl)cyclobutyl]-5-methoxy-1,3-benzothiazol-6-yl]-6-(trifluoromethyl) pyridine-2-carboxamide (120 mg, 274 umol) in THF (2 mL) was added methane sulfonic anhydride (477 mg, 2.74 mmol) and TEA (83.2 mg, 822 umol). The mixture was then stirred at 25° C. for 16 h. On completion, the reaction mixture was diluted with water (5 ml), then extracted with EA (3×5 ml). The combined organic layers were concentrated in vacuo to give the title compound as a yellow solid (140 mg, 99% yield). ¹H NMR (400 MHz, CDCl₃) δ 10.64 (s, 1H), 9.04 (s, 1H), 8.43 (d, J=8.0 Hz, 1H), 8.07 (t, J=7.6 Hz, 1H), 7.81 (d, J=8.0 Hz, 1H), 7.50 (s, 1H), 4.30 (d, J=6.4 Hz, 2H), 3.99 (s, 3H), 3.91 (s, 1H), 3.00 (s, 3H), 2.92-2.81 (m, 1H), 2.68-2.57 (m, 2H), 2.41 (m, 2H).

### Step 3—N-(2-((1r, 3r)-3-(iodomethyl)cyclobutyl)-5-methoxybenzo[d]thiazol-6-yl)-6-(trifluoromethyl) picolinamide

To a solution of [3-[5-methoxy-6-[[6-(trifluoromethyl)pyridine-2-carbonyl]amino]-1,3-ben zothiazol-2-yl]cyclobutyl]methyl methane sulfonate (140 mg, 271 umol) in THF (3 mL) was added NaI (203 mg, 1.36 mmol) and TEA (54.9 mg, 543 umol). The reaction mixture was then stirred at 75° C. for 16 h. On completion, the reaction mixture was diluted with water (5 ml), then extracted with EA (3×5 ml). The combined organic layers were concentrated in vacuo to give the title compound as a yellow solid (140 mg, 94% yield). ¹H NMR (400 MHz, CDCl₃) δ 10.63 (s, 1H), 9.03 (s, 1H), 8.43 (d, J=8.0 Hz, 1H), 8.07 (t, J=8.0 Hz, 1H), 7.81 (d, J=7.6 Hz, 1H), 7.47 (s, 1H), 3.99 (s, 3H), 3.86-3.72 (m, 1H), 3.34 (d, J=7.6 Hz, 2H), 2.93-2.79 (m, 1H), 2.62-2.48 (m, 2H), 2.24 (m, 2H).

### N-[6-methoxy-2-(4-piperidyl) indazol-5-yl]-6-(trifluoromethyl) pyridine-2-carboxamide (Intermediate CAE)

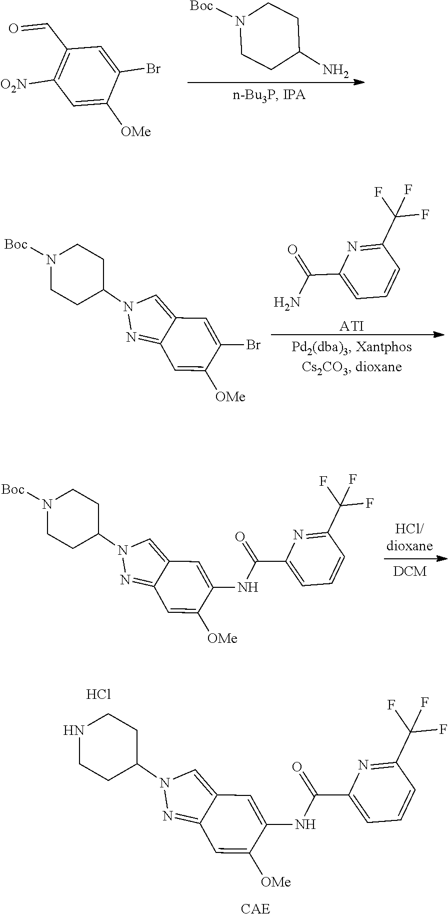

### Step 1—Tert-butyl 4-(5-bromo-6-methoxy-indazol-2-yl) piperidine-1-carboxylate

A solution of tert-butyl4-aminopiperidine-1-carboxylate (462 mg, 2.31 mmol, CAS #502482-34-0) and 5-bromo-4-methoxy-2-nitro-benzaldehyde (500 mg, 1.92 mmol, synthesized via Steps 1-2 of Intermediate ATE) in IPA (10 mL) was stirred at 80° C. for 16 hrs. Then the reaction mixture was cooled to 25° C., and tributylphosphane (1.17 g, 5.77 mmol) was added to the solution which was then stirred at 80° C. for 6 hrs. On completion, the mixture was concentrated in vacuo. The crude product was purified by reversed phase (0.1% FA condition) to give the title compound (610 mg, 77% yield) as a white solid. ¹H NMR (400 MHz, DMSO-d₆) δ 8.32 (s, 1H), 7.96 (s, 1H), 7.11 (s, 1H), 4.69-4.58 (m, 1H), 4.07 (d, J=11.6 Hz, 2H), 3.86 (s, 3H), 2.94 (s, 2H), 2.11-2.04 (m, 2H), 1.97-1.85 (m, 2H), 1.42 (s, 9H). LC-MS (ESI⁺) m/z 412.0 (M+H)⁺.

### Step 2—Tert-butyl 4-[6-methoxy-5-[[6-(trifluoromethyl) pyridine-2-carbonyl]amino]indazol-2-yl]piperidine-1-carboxylate

To a solution of tert-butyl 4-(5-bromo-6-methoxy-indazol-2-yl)piperidine-1-carboxylate (550 mg, 1.34 mmol) and 6-(trifluoromethyl)pyridine-2-carboxamide (305 mg, 1.61 mmol, CAS #22245-84-7, Intermediate ATI) in dioxane (1 mL) was added Pd₂(dba)₃ (122 mg, 134 umol), Xantphos (155 mg, 268 umol) and Cs₂CO₃ (873 mg, 2.68 mmol). Then the mixture was degassed and purged with N₂ three times, and the mixture was stirred at 100° C. for 4 hrs under N₂ atmosphere. On completion, the mixture was filtered through a pad of Celite and the pad of filter cake was washed with EA, then the filter liquor was concentrated in vacuo. The crude product was purified by reversed phase (0.1% FA condition) to give the title compound (686 mg, 98% yield) as an orange solid. ¹H NMR (400 MHz, DMSO-d₆) δ 10.50 (s, 1H), 8.69 (s, 1H), 8.49-8.43 (m, 1H), 8.43-8.36 (m, 2H), 8.21 (dd, J=1.2, 7.6 Hz, 1H), 7.16 (s, 1H), 4.70-4.50 (m, 1H), 4.08 (d, J=11.6 Hz, 2H), 3.98 (s, 3H), 3.06-2.86 (m, 2H), 2.17-2.05 (m, 2H), 2.00-1.86 (m, 2H), 1.43 (s, 9H). LC-MS (ESI⁺) m/z 520.1 (M+H)⁺.

### Step 3—N-[6-methoxy-2-(4-piperidyl) indazol-5-yl]-6-(trifluoromethyl) pyridine-2-carboxamide

To a solution of tert-butyl 4-[6-methoxy-5-[[6-(trifluoromethyl) pyridine-2-carbonyl]amino]indazol-2-yl]piperidine-1-carboxylate (60 mg, 115 umol) in DCM (1 mL) was added HCl/dioxane (4 M, 1 mL). The mixture was then stirred at 25° C. for 4 hrs. On completion, the mixture was concentrated in vacuo to give the title compound (52 mg, 98% yield, HCl) as a pink solid. ¹HNMR (400 MHz, DMSO-d₆) δ 10.51 (s, 1H), 9.15-9.01 (m, 1H), 8.71 (s, 1H), 8.49-8.44 (m, 1H), 8.42 (d, J=7.6 Hz, 1H), 8.40-8.36 (m, 1H), 8.22 (dd, J=1.2, 7.6 Hz, 1H), 7.19 (s, 1H), 4.83-4.71 (m, 1H), 3.99 (s, 3H), 3.44 (d, J=12.0 Hz, 2H), 3.20-3.05 (m, 2H), 2.34-2.25 (m, 4H). LC-MS (ESI⁺) m/z 420.6 (M+H)⁺.

### Tert-butyl-dimethyl-[[4-(4, 4, 5, 5-tetramethyl-1, 3, 2-dioxaborolan-2-yl) cyclohex-3-en-1-yl]methoxy]silane (Intermediate CAF)

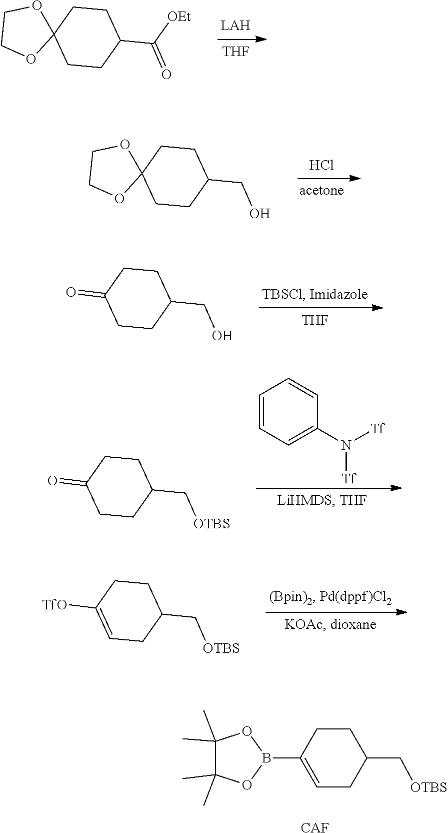

### Step 1—1, 4-Dioxaspiro [4.5]decan-8-ylmethanol

To a solution of LiAlH₄ (3.19 g, 84.0 mmol) in THF (50 mL) was added a solution of ethyl 1, 4-dioxaspiro[4.5]decane-8-carboxylate (15 g, 70.0 mmol, CAS #1489-97-0) in THF (90 mL) dropwise at 0° C. The mixture was stirred at 0° C. for 1 hr. On completion, the mixture was quenched with H₂O (3.2 mL), followed by 15% NaOH (3.2 mL), and H₂O (9.6 mL). Then the mixture was dried over anhydrous Na₂SO₄, filtered and concentrated in vacuo to give the title compound (11 g, 91% yield) as colorless oil. ¹H NMR (400 MHz, CDCl₃) δ 4.01-3.89 (m, 4H), 3.50 (d, J=6.4 Hz, 2H), 1.79 (d, J=10.0 Hz, 4H), 1.61-1.51 (m, 3H), 1.35-1.19 (m, 2H).

### Step 2—4-(Hydroxymethyl) cyclohexanone

To a solution of 1, 4-dioxaspiro [4.5]decan-8-ylmethanol (11 g, 63.8 mmol) in acetone (70 mL) was added HCl (2 M, 24.5 mL). The mixture was then stirred at 25° C. for 13 hrs. On completion, the mixture was adjusted to pH=8 with saturated NaHCO₃, then extracted with DCM (5×80 mL). The organic layers were dried over anhydrous Na₂SO₄, filtered and concentrated in vacuo to give the title compound (8.0 g, 97% yield) as yellow oil. ¹H NMR (400 MHz, CDCl₃) δ 3.58 (d, J=6.4 Hz, 2H), 2.52-2.28 (m, 4H), 2.19-2.08 (m, 2H), 2.03-1.89 (m, 1H), 1.52-1.38 (m, 2H).

### Step 3—4-[[Tert-butyl (dimethyl) silyl]oxymethyl]cyclohexanone

To a solution of 4-(hydroxymethyl) cyclohexanone (7.7 g, 60.0 mmol) and imidazole (8.18 g, 120 mmol) in THF (80 mL) was added TBSCl (10.8 g, 72.0 mmol) and DMF (439 mg, 6.01 mmol). The mixture was then stirred at 25° C. for 2 hrs. On completion, the mixture was quenched with H₂O (40 mL) and the organic layer was concentrated in vacuo. The residue was purified by silica gel column (PE:EA=10:1) to give the title compound (13.5 g, 92% yield) as colorless oil. ¹H NMR (400 MHz, CDCl₃) δ 3.52 (d, J=6.4 Hz, 2H), 2.48-2.27 (m, 4H), 2.16-2.03 (m, 2H), 1.99-1.82 (m, 1H), 1.47-1.40 (m, 2H), 0.90 (s, 9H), 0.06 (s, 6H).

### Step 4—[4-[[Tert-butyl (dimethyl) silyl]oxymethyl] cyclohexen-1-yl]trifluoromethanesulfonate

To a solution of 4-[[tert-butyl (dimethyl) silyl]oxymethyl]cyclohexanone (5 g, 20.6 mmol) in THF (50 mL) was added LiHMDS (1 M, 30.9 mL) at −70° C. The mixture was then stirred at −70° C. for 2 hrs. Then a solution of 1, 1, 1-trifluoro-Nphenyl-N-(trifluoromethylsulfonyl) methanesulfonamide (8.84 g, 24.7 mmol, CAS #37595-74-7) in THF (20 mL) was added to above solution dropwise. Then the mixture was warm to 20° C. and stirred for 2 hrs. On completion, the mixture was quenched with saturated NH₄Cl (10 mL) and extracted with EA (2×5 mL). The organic layer was washed with brine (2×10 mL), dried over anhydrous Na₂SO₄, filtered and concentrated in vacuo. The residue was purified by silica gel column (PE/EA=100/1) to give the title compound (6.75 g, 87% yield) as colorless oil. ¹H NMR (400 MHz, CDCl₃) δ 5.8-5.7 (m, 1H), 3.51 (d, J=6.0 Hz, 2H), 2.44-2.15 (m, 3H), 2.01-1.85 (m, 2H), 1.84-1.75 (m, 1H), 1.52-1.41 (m, 1H), 0.90 (s, 9H), 0.04 (s, 6H).

### Step 5—Tert-butyl-dimethyl-[[4-(4, 4, 5, 5-tetramethyl-1, 3, 2-dioxaborolan-2-yl) cyclohex-3-en-1-yl]methoxy]silane

To a solution of [4-[[tert-butyl (dimethyl) silyl]oxymethyl]cyclohexen-1-yl]trifluoromethanesulfonate (6.75 g, 18.0 mmol), 4, 4, 5, 5-tetramethyl-2-(4, 4, 5, 5-tetramethyl-1, 3, 2-dioxaborolan-2-yl)-1, 3, 2-dioxaborolane (4.58 g, 18.0 mmol) and KOAc (5.31 g, 54.0 mmol) in dioxane (80 mL) was added Pd(dppf)Cl₂ (1.32 g, 1.80 mmol) under N₂. The mixture was then stirred at 65° C. for 16 hrs. On completion, the mixture was concentrated in vacuo. The residue was purified by silica gel column (PE/EA=100/1) to give the title compound (3.7 g, 58% yield) as a white solid. ¹H NMR (400 MHz, CDCl₃) δ 6.61-6.53 (m, 1H), 3.48 (d, J=6.0 Hz, 2H), 2.29-2.14 (m, 2H), 2.13-1.80 (m, 2H), 1.80-1.70 (m, 2H), 1.27 (s, 12H), 1.25-1.22 (m, 1H), 0.89 (s, 9H), 0.04 (s, 6H).

### 4-[1-(2, 6-Dioxo-3-piperidyl)-3-methyl-2-oxo-benzimidazol-4-yl]cyclohexanecarbaldehyde (Intermediate CAG)

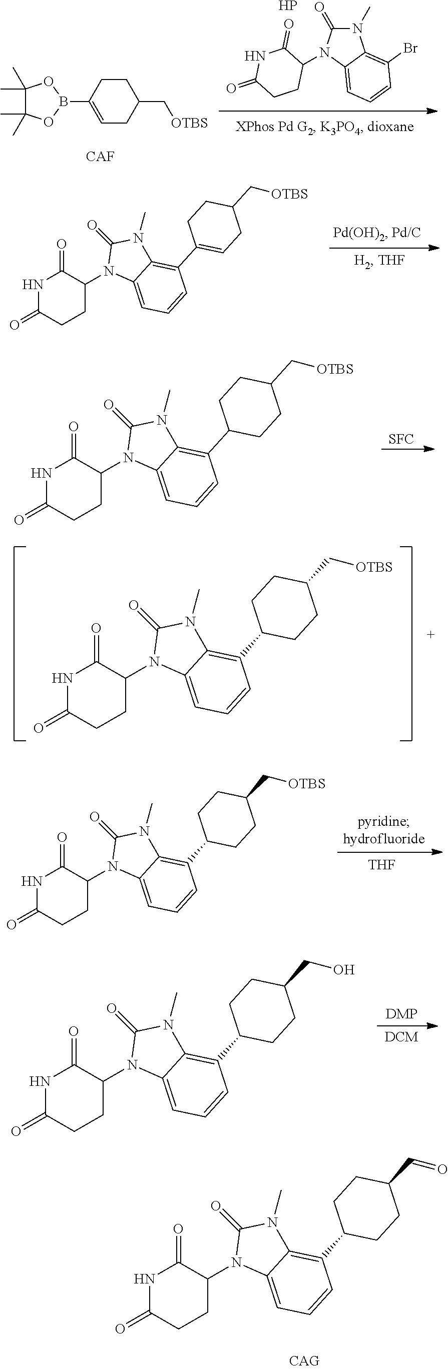

### Step 1—3-[4-[4-[[Tert-butyl (dimethyl) silyl] oxymethyl] cyclohexen-1-yl]-3-methyl-2-oxo-benzimidazol-1-yl]piperidine-2, 6-dione

To a solution of tert-butyl-dimethyl-[[4-(4, 4, 5, 5-tetramethyl-1, 3, 2-dioxaborolan-2-yl) cyclohex-3-en-1-yl]methoxy]silane (1.35 g, 3.84 mmol, Intermediate CAF), 3-(4-bromo-3-methyl-2-oxo-benzimidazol-1-yl)piperidine-2,6-dione (1 g, 2.96 mmol, Intermediate HP) and K₃PO₄ (1.88 g, 8.87 mmol) in dioxane (13 mL) was added XPHOS-PD-G2 (232 mg, 295 umol) under N₂. The mixture was stirred at 80° C. for 16 hrs under N₂. On completion, the mixture was filtered and concentrated in vacuo. The residue was purified by prep-HPLC (reverse phase: 0.1% FA) to give the title compound (600 g, 41% yield) as a yellow solid. ¹H NMR (400 MHz, DMSO-d₆) δ 11.09 (s, 1H), 7.09-6.91 (m, 2H), 6.77 (dd, J=1.2, 7.2 Hz, 1H), 5.70-5.60 (m, 1H), 5.37 (dd, J=5.2, 12.4 Hz, 1H), 3.61-3.48 (m, 2H), 3.32 (s, 3H), 2.96-2.80 (m, 1H), 2.75-2.66 (m, 1H), 2.65-2.56 (m, 1H), 2.41-2.32 (m, 1H), 2.30-2.17 (m, 2H), 2.04-1.97 (m, 1H), 1.92-1.74 (m, 3H), 1.44-1.37 (m, 1H), 0.88 (s, 9H), 0.11 (s, 6H). LC-MS (ESI⁺) m/z 484.5 (M+H)⁺.

### Step 2—3-[4-[4-[[Tert-butyl (dimethyl) silyl]oxymethyl] cyclohexyl]-3-methyl-2-oxo-benzimidazol-1-yl] piperidine-2,6-dione

To a solution of 3-[4-[4-[[tert-butyl(dimethyl)silyl]oxymethyl]cyclohexen-1-yl]-3-methyl-2-oxo-benzimidazol-1-yl]piperidine-2,6-dione (500 mg, 1.03 mmol) in THF (10 mL) was added Pd/C (300 mg, 10 wt %) and Pd(OH)₂ (300 mg, 213 umol, 10 wt %). The mixture was stirred at 60° C. for 16 hrs under H₂ (15 psi). On completion, the mixture was filtered and concentrated in vacuo to give the title compound (500 mg, 99% yield) as a white solid. ¹H NMR (400 MHz, DMSO-d₆) δ 11.08 (s, 1H), 7.05-6.98 (m, 2H), 6.97-6.92 (m, 1H), 5.36 (dd, J=5.2, 12.4 Hz, 1H), 3.71 (d, J=7.6 Hz, 1H), 3.60-3.54 (m, 3H), 3.45 (d, J=6.4 Hz, 1H), 3.27-3.15 (m, 1H), 2.96-2.82 (m, 1H), 2.76-2.67 (m, 1H), 2.65-2.57 (m, 1H), 2.03-1.94 (m, 1H), 1.92-1.71 (m, 4H), 1.70-1.59 (m, 4H), 1.58-1.47 (m, 1H), 0.89 (s, 9H), 0.01 (s, 6H). LC-MS (ESI⁺) m/z 486.2 (M+H)⁺.

### Step 3—3-(4-((1r, 4r)-4-(((tert-butyldimethylsilyl)oxy)methyl)cyclohexyl)-3-methyl-2-oxo-2,3-dihydro-1H-benzo[d]imidazol-1-yl)piperidine-2,6-dione & 3-(4-((1s, 4s)-4-(((tert-butyl dimethylsilyl)oxy)methyl)cyclohexyl)-3-methyl-2-oxo-2,3-dihydro-1H-benzo[d]imidazol-1-yl) piperidine-2,6-dione

3-[4-[4-[[tert-butyl (dimethyl) silyl]oxymethyl]cyclohexyl]-3-methyl-2-oxo-benzimidazol-1-yl]piperidine-2, 6-dione (590 mg, 1.21 mmol) was separated by SFC (column: DAICEL CHIRALPAK AD (250 mm*30 mm, 10 um); mobile phase: [0.1% NH₃H₂O IPA]; B %: 25%-25%, 4; 90 min), then purified by prep-HPLC (reverse phase: 0.1% FA) to give 3-[4-[4-[[Tert-butyl (dimethyl) silyl]oxymethyl]cyclohexyl]-3-methyl-2-oxo-benzimidazol-1-yl]piperidine-2, 6-dione (50 mg, 8% yield) as a white solid (¹H NMR (400 MHz, CDCl₃) δ 7.98 (s, 1H), 7.02-6.93 (m, 2H), 6.64-6.52 (m, 1H), 5.23-5.06 (m, 1H), 3.62 (s, 3H), 3.41 (d, J=6.0 Hz, 2H), 3.16-3.02 (m, 1H), 2.93-2.60 (m, 3H), 2.18-2.10 (m, 1H), 1.89 (d, J=11.2 Hz, 4H), 1.53-1.46 (m, 3H), 1.12-0.98 (m, 2H), 0.85 (s, 9H), 0.00 (s, 6H). LC-MS (ESI⁺) m/z 486.3 (M+H)⁺) and 3-[4-[4-[[tert-butyl (dimethyl) silyl]oxymethyl]cyclohexyl]-3-methyl-2-oxobenzimidazol-piperidine-2,6-dione (200 mg, 34% yield) as a white solid (¹H NMR (400 MHz, CDCl₃) δ 8.20 (s, 1H), 7.11-6.97 (m, 2H), 6.71-6.59 (m, 1H), 5.30-5.13 (m, 1H), 3.78-3.66 (m, 5H), 3.32-3.18 (m, 1H), 2.99-2.68 (m, 3H), 2.26-2.18 (m, 1H), 2.01-1.84 (m, 3H), 1.78-1.58 (m, 6H), 0.93 (s, 9H), 0.09 (s, 6H). LC-MS (ESI⁺) m/z 486.3 (M+H)⁺). The cis/trans isomers were confirmed by 2D NMR.

### Step 4—3-(4-((1r, 4r)-4-(hydroxymethyl)cyclohexyl)-3-methyl-2-oxo-2,3-dihydro-1H-benzo[d]imidazol-1-yl)piperidine-2,6-dione

To a solution of 3-[4-[4-[[tert-butyl(dimethyl) silyl]oxymethyl]cyclohexyl]-3-methyl-2-oxo-benzimidazol-1-yl]piperidine-2, 6-dione (50 mg, 102 umol) in THF (2 mL) was added pyridine; hydrofluoride (550 mg, 5.55 mmol). The mixture was stirred at 25° C. for 0.5 hr. On completion, the mixture was concentrated in vacuo. The crude product was purified by reversed phase (0.1% FA condition) to give the title compound (30 mg, 78% yield) as a white solid. LC-MS (ESI⁺) m/z 372.1 (M+H)⁺.

### Step 5—(1r, 4r)-4-(1-(2,6-dioxopiperidin-3-yl)-3-methyl-2-oxo-2,3-dihydro-1H-benzo[d]imidazol-4-yl)cyclohexane-1-carbaldehyde

To a solution of 3-[4-[4-(hydroxymethyl) cyclohexyl]-3-methyl-2-oxo-benzimidazol-1-yl]piperidine-2, 6-dione (15 mg, 40.3 umol) in DCM (2 mL) was added DMP (25.6 mg, 60.5 umol). The mixture was stirred at 25° C. for 1 hr. On completion, the mixture was diluted with DCM (100 mL), quenched with saturated Na₂S₂O₃ (10 mL) and washed with saturated NaHCO₃ (2×15 mL). The organic layer was dried over anhydrous Na₂SO₄, filter and concentrated in vacuo to give the title compound (20 mg, 100% yield) as a white solid. LC-MS (ESI⁺) m/z 370.3 (M+H)⁺.

### N-[7-fluoro-2-(4-formylcyclohexyl)indazol-5-yl]-6-(trifluoromethyl)pyridine-2-carboxamide (Intermediate CAH)

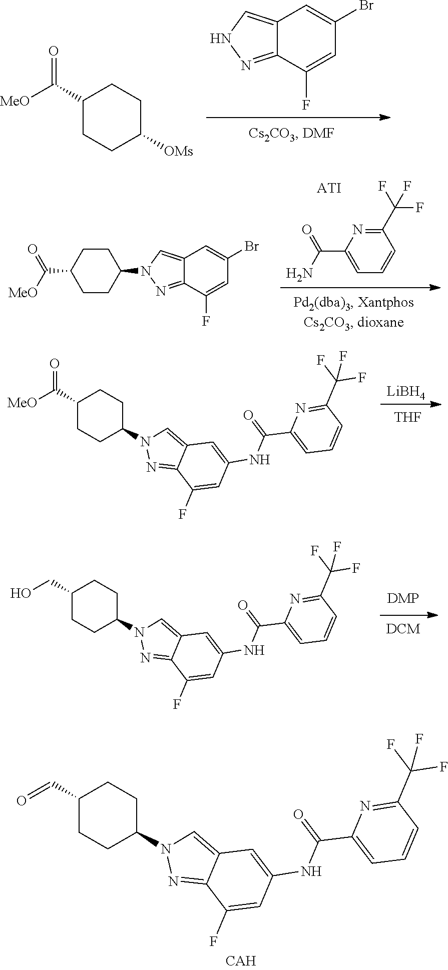

### Step 1—Methyl-4-(5-bromo-7-fluoro-indazol-2-yl)cyclohexanecarboxylate

To a solution of 5-bromo-7-fluoro-2H-indazole (404 mg, 1.88 mmol, CAS #1260381-83-6) and methyl 4-methylsulfonyloxycyclohexanecarboxylate (890 mg, 3.77 mmol, synthesized via Step 1 of Intermediate QS) in DMF (10 mL) was added Cs₂CO₃ (1.23 g, 3.77 mmol). The mixture was then stirred at 80° C. for 16 hrs. On completion, the reaction mixture was filtered and concentrated under reduced pressure to give a residue, and the residue was purified by reversed-phase HPLC (0.10% FA condition) to give the title compound (120 mg, 17% yield) as yellow solid. LC-MS (ESI⁺) m/z 355.0 (M+1)⁺. ¹H NMR (400 MHz, CDCl₃) δ 7.95 (d, J=2.8 Hz, 1H), 7.60 (d, J=1.6 Hz, 1H), 7.06 (dd, J=1.6, 10.4 Hz, 1H), 4.48-4.40 (m, 1H), 3.72 (s, 3H), 2.50-2.42 (m, 1H), 2.39-2.34 (m, 2H), 2.28-2.22 (m, 2H), 2.07-1.97 (m, 2H), 1.76-1.65 (m, 2H).

### Step 2—Methyl-4-[7-fluoro-5-[[6-(trifluoromethyl)pyridine-2-carbonyl]amino]indazol-2-yl]cyclohexanecarboxylate

A mixture of methyl 4-(5-bromo-7-fluoro-indazol-2-yl)cyclohexanecarboxylate (100 mg, 281 umol), 6-(trifluoromethyl)pyridine-2-carboxamide (64.2 mg, 337 umol, CAS #22245-84-7, Intermediate ATI), Pd₂(dba)₃ (25.7 mg, 28.1 umol), Xantphos(16.2 mg, 28.1 umol) and Cs₂CO₃ (183 mg, 563 umol) in dioxane (5 mL) was degassed and purged with N₂ three times. Then the mixture was stirred at 80° C. for 16 hours under N₂ atmosphere. On completion, the reaction mixture was filtered and concentrated under reduced pressure to give a residue. The residue was purified by prep-HPLC (column: Phenomenex luna C18 150*25 mm*10 um; mobile phase: [water(0.225% FA)-ACN]; B %: 50%-80%, 11.5 min) to give the title compound (129 mg, 95% yield) as a white solid. LC-MS (ESI⁺) m/z 465.1 (M+H)⁺. ¹H NMR (400 MHz, DMSO-d₆) δ 10.47 (s, 1H), 8.55 (d, J=2.8 Hz, 1H), 8.42-8.34 (m, 2H), 8.21-8.13 (m, 2H), 7.56 (dd, J=1.2, 13.2 Hz, 1H), 4.59-4.51 (m, 1H), 3.64 (s, 3H), 3.30 (s, 1H), 2.21-2.17 (m, 2H), 2.13-2.06 (m, 2H), 2.05-1.94 (m, 2H), 1.66-1.56 (m, 2H).

### Step 3—N-[7-fluoro-2-[4-(hydroxymethyl)cyclohexyl]indazol-5-yl]-6-(trifluoromethyl)pyridine-2-carboxamide

A mixture of methyl 4-[7-fluoro-5-[[6-(trifluoromethyl)pyridine-2-carbonyl]amino]indazol-2-yl]cyclohexanecarboxylate (100 mg, 215 umol) in THF (2 mL) and MeOH (0.25 mL) was degassed and purged with N₂ three times. To the mixture was added LiBH4 (75.0 mg, 3.45 mmol), and then the mixture was stirred at 60° C. for 36 hours under N₂ atmosphere. On completion, the reaction mixture was quenched with H₂O (4 mL) at 25° C., and then extracted with EA (3×3 mL). The combined organic layers were washed with brine (2×3 mL), dried over Na₂SO₄, filtered and concentrated under reduced pressure to give the title compound (81 mg, 83% yield) as white solid. LC-MS (ESI⁺) m/z 437.1 (M+H)⁺. ¹H NMR (400 MHz, DMSO-d₆) δ 10.46 (s, 1H), 8.56 (d, J=2.8 Hz, 1H), 8.45-8.32 (m, 2H), 8.22-8.12 (m, 2H), 7.54 (d, J=13.6 Hz, 1H), 4.51-4.39 (m, 2H), 3.31-3.26 (m, 3H), 2.18-2.15 (m, 2H), 1.94-1.86 (m, 4H), 1.19-1.15 (m, 2H).

### Step 4—N-[7-fluoro-2-(4-formylcyclohexyl)indazol-5-yl]-6-(trifluoromethyl)pyridine-2-carboxamide

To a solution of N-[7-fluoro-2-[4-(hydroxymethyl)cyclohexyl]indazol-5-yl]-6-(trifluoromethyl) pyridine-2-carboxamide (60.0 mg, 137 umol) in DCM (2 mL) was added DMP (139 mg, 329 umol) at 0° C. The mixture was then stirred at 25° C. for 4 hours. On completion, the reaction mixture was quenched with Na₂S₂O₃ (1 mL) at 25° C., and then diluted with NaHCO₃ (5 mL) and extracted with DCM (3×10 mL). The combined organic layers were washed with brine (2×10 mL), dried over Na₂SO₄, filtered and concentrated under reduced pressure to give the title compound (59 mg, 90% yield) as a white solid LC-MS (ESI⁺) m/z 435.1 (M+H)⁺.

### N-[2-(4-formylcyclohexyl)pyrazolo[1,5-a]pyridin-5-yl]-6-(trifluoromethyl)pyridine-2-carboxamide (Intermediate CAI)

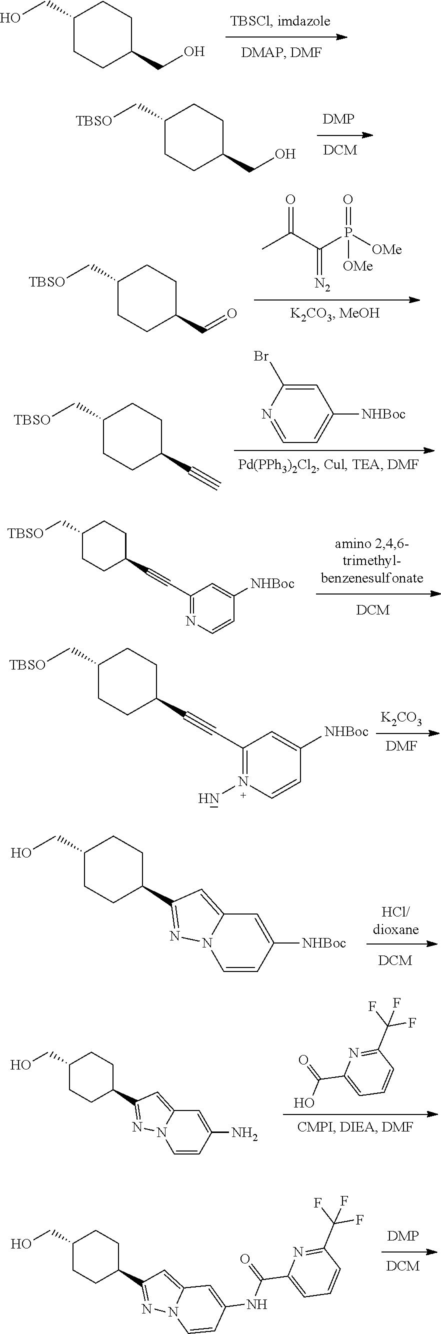

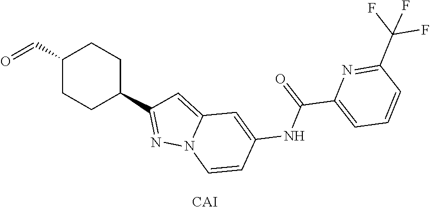 [SMILES: O=C[C@H]1CC[C@H](c2cc3cc(NC(=O)c4cccc(C(F)(F)F)n4)ccn3n2)CC1]

### Step 1—[4-[[Tert-butyl(dimethyl)silyl]oxymethyl]cyclohexyl]methanol

To a solution of [4-(hydroxymethyl)cyclohexyl]methanol (20 g, 138 mmol, CAS #3236-48-4) in DMF (150 mL) was added TBSCI (16.7 g, 110 mmol), imidazole (18.8 g, 277 mmol) and DMAP (1.69 g, 13.8 mmol). The mixture was stirred at 25° C. for 16 hrs. On completion, the reaction mixture was quenched with H₂O (150 mL) and extracted with EA (2×300 mL). The combined organic phase was brine (2×150 mL), dried with anhydrous Na₂SO₄, filtered and concentrated in vacuo to give a residue. The residue was purified by silica gel chromatography (PE:EA=20:1 to 5:1, Rf=0.5) to give the title compound (10.9 g, 30% yield) as yellow oil. ¹H NMR (400 MHz, CDCl₃) δ 3.47 (d, J=6.4 Hz, 2H), 3.42 (d, J=6.4 Hz, 2H), 1.88-1.76 (m, 4H), 1.44-1.41 (m, 2H), 1.03-0.93 (m, 4H), 0.91-0.88 (m, 9H), 0.09-0.02 (m, 6H).

### Step 2—4-[[Tert-butyl(dimethyl)silyl]oxymethyl]cyclohexanecarbaldehyde

To a solution of [4-[[tert-butyl(dimethyl)silyl]oxymethyl]cyclohexyl]methanol (2.4 g, 9.29 mmol) in DCM (20 mL) was added DMP (5.12 g, 12 mmol). The reaction mixture was stirred at 25° C. for 2 hrs. On completion, the reaction mixture was quenched with Na₂S₂O₃ (20 mL) and saturated NaHCO₃ (20 mL) at 25° C., and then stirred for 15 minutes. On completion, the reaction mixture was extracted with DCM (2×30 mL), then the combined organic layers was washed with saturated NaCl (2×15 mL). Then the combined organic layers was dried over Na₂SO₄, filtered and concentrated in vacuo to give title compound (2.3 g, 96% yield) was obtained as yellow oil. ¹H NMR (400 MHz, CDCl₃) δ 9.63 (s, 1H), 3.45-3.40 (m, 2H), 2.23-2.10 (m, 1H), 2.07-1.99 (m, 2H), 1.90 (dd, J=2.8, 13.2 Hz, 2H), 1.48-1.42 (m, 1H), 1.33-1.22 (m, 2H), 1.05-0.93 (m, 2H), 0.90 (s, 9H), 0.05 (s, 6H).

### Step 3—Tert-butyl-[(4-ethynylcyclohexyl)methoxy]-dimethyl-silane

To a solution of 4-[[tert-butyl(dimethyl)silyl]oxymethyl]cyclohexanecarbaldehyde (2.3 g, 8.97 mmol) and K₂CO₃ (2.48 g, 17.9 mmol) in MeOH (15 mL) was added 1-diazo-1-dimethoxyphosphoryl-propan-2-one (2.58 g, 13.4 mmol, CAS #90965-06-3) at 0° C. The reaction mixture was stirred at 25° C. for 12 hrs. On completion, the reaction mixture was quenched with water (15 mL) at 25° C., and then stirred for 30 minutes. The reaction mixture was extracted with EA (2×20 mL) then the organic layers were dried and concentrated in vacuo to give the residue. The residue was purified by column chromatography (SiO₂, PE:EA=1:0 to PE:EA=100:1, PE:EA=50:1, P1:Rf=0.4) to give the title compound (1.3 g, 57% yield) as yellow oil. ¹H NMR (400 MHz, CDCl₃) δ 3.39 (d, J=6.4 Hz, 2H), 2.18 (m, 1H), 2.05 (d, J=2.4 Hz, 1H), 2.04-1.97 (m, 2H), 1.84-1.75 (m, 2H), 1.49-1.43 (m, 1H), 1.43-1.32 (m, 2H), 0.99-0.92 (m, 2H), 0.90 (s, 9H), 0.10-0.00 (m, 6H).

### Step 4—Tert-butyl N-[2-[2-[4-[[tert-butyl(dimethyl)silyl]oxymethyl]cyclohexyl]ethynyl]-4-pyridyl] carbamate

To a solution of tert-butyl-[(4-ethynylcyclohexyl)methoxy]-dimethyl-silane (1 g, 3.96 mmol), tert-butyl N-(2-bromo-4-pyridyl)carbamate (721 mg, 2.64 mmol, CAS #433711-95-6), Pd(PPh₃)₂Cl₂ (185 mg, 264 umol) and CuI (100 mg, 528 umol) in DMF (10 mL) was added DIEA (1.71 g, 13.2 mmol). The mixture was stirred at 80° C. for 12 hrs. On completion, the reaction mixture was filtered and concentrated in vacuo to give a residue. The residue was purified by reverse phase (0.1% FA condition) and purified by column chromatography (SiO₂, PE:EA=15:1 to PE:EA=5:1, PE:EA=2:1, P1:Rf=0.27) to give the title compound (434 mg, 37% yield) as a yellow solid. ¹H NMR (400 MHz, DMSO-d₆) δ 9.88 (s, 1H), 8.26 (s, 1H), 7.51 (s, 1H), 7.31 (dd, J=1.6, 5.6 Hz, 1H), 3.38 (d, J=6.4 Hz, 2H), 2.46-2.39 (m, 1H), 2.02-1.97 (m, 2H), 1.74 (dd, J=2.8, 13.2 Hz, 2H), 1.48 (s, 9H), 1.44-1.31 (m, 3H), 1.02-0.90 (m, 2H), 0.86 (s, 9H), 0.09-0.03 (m, 6H); LC-MS (ESI⁺) m/z 445.4 (M+H)⁺.

### Step 5—[4-(Tert-butoxycarbonylamino)-2-[2-[4-[[tert-butyl(dimethyl)silyl]oxymethyl]cyclohexyl]ethynyl]pyridin-1-ium-1-yl]azanide

To a solution of tert-butyl N-[2-[2-[4-[[tert-butyl(dimethyl)silyl]oxymethyl]cyclohexyl]ethynyl]-4-pyridyl]carbamate (228 mg, 512 umol) in DCM (4 mL) was added amino 2,4,6-trimethylbenzenesulfonate (165 mg, 769 umol). The reaction mixture was stirred at 25° C. for 48 hrs. On completion, the mixture was concentrated in vacuo to give the title compound (235 mg, 100% yield) as a gray solid. LC-MS (ESI⁺) m/z 460.3 (M+H)⁺.

### Step 6—Tert-butyl N-[2-[4-(hydroxymethyl)cyclohexyl]pyrazolo[1,5-a]pyridin-5-yl]carbamate

To a solution of [4-(tert-butoxycarbonylamino)-2-[2-[4-[[tert-butyl(dimethyl)silyl]oxymethyl]cyclohexyl]ethynyl]pyridin-1-ium-1-yl]azanide (200 mg, 435 umol) in DMF (2 mL) was added K₂CO₃ (120 mg, 870 umol). The mixture was stirred at 25° C. for 24 hrs. On completion, the reaction mixture was filtered and concentrated in vacuo to give a residue. The residue was purified by prep-HPLC (column: Phenomenex luna C18 150*25 mm*10 um; mobile phase: [water (0.225% FA)-ACN]; B %: 35%-65%, 10 min) to give the title compound (23 mg, 14% yield) as a white solid. ¹H NMR (400 MHz, DMSO-d₆) δ 9.59 (s, 1H), 8.40 (d, J=7.6 Hz, 1H), 7.69 (s, 1H), 6.76 (dd, J=2.4, 7.6 Hz, 1H), 6.20 (s, 1H), 4.40 (t, J=5.2 Hz, 1H), 3.25 (t, J=5.6 Hz, 2H), 2.66-2.57 (m, 1H), 2.05-1.97 (m, 2H), 1.87-1.80 (m, 2H), 1.49 (s, 9H), 1.46-1.38 (m, 3H), 1.08-0.97 (m, 2H); LC-MS (ESI⁺) m/z 346.2 (M+H)⁺.

### Step 7—[4-(5-Aminopyrazolo[1,5-a]pyridin-2-yl)cyclohexyl]methanol

To a solution of tert-butyl N-[2-[4-(hydroxymethyl)cyclohexyl]pyrazolo[1,5-a]pyridin-5-yl]carbamate (23 mg, 66.5 umol) in DCM (1 mL) was added HCl/dioxane (4 M, 16.6 uL). The reaction mixture was stirred at 25° C. for 2 hrs. On completion, the reaction mixture was concentrated in vacuo to give the title compound (18 mg, 95% yield, HCl) as a yellow solid. LC-MS (ESI⁺) m/z 246.1 (M+H)⁺.

### Step 8—N-[2-[4-(hydroxymethyl)cyclohexyl]pyrazolo[1,5-a]pyridin-5-yl]-6-(trifluoromethyl)pyridine-2-carboxamide

To a solution of 6-(trifluoromethyl)pyridine-2-carboxylic acid (9.77 mg, 51.1 umol, CAS #131747-42-7) in DMF (1 mL) was added dropwise DIEA (41.2 mg, 319 umol) and CMPI (19.5 mg, 76.6 umol) at 25° C. After addition, the mixture was stirred at this temperature for 30 min, and then [4-(5-aminopyrazolo[1,5-a]pyridin-2-yl)cyclohexyl]methanol (18 mg, 63.8 umol, HCl) in DMF (1 mL) was added dropwise at 25° C. The resulting mixture was stirred at 25° C. for 2 hrs. On completion, the reaction mixture was quenched with water (0.05 mL) and concentrated in vacuo to give a residue. The residue was diluted with water (4 mL) and extracted with EA (2×5 mL). The combined organic layer was dried over Na₂SO₄, filtered and concentrated in vacuo to give the title compound (26 mg, 77% yield) as a yellow solid. ¹H NMR (400 MHz, DMSO-d₆) δ 10.61 (s, 1H), 8.55 (d, J=7.6 Hz, 1H), 8.43-8.34 (m, 2H), 8.25-8.15 (m, 2H), 7.25 (dd, J=2.4, 7.6 Hz, 1H), 6.37 (s, 1H), 4.40 (s, 1H), 3.26 (d, J=5.6 Hz, 2H), 2.71-2.63 (m, 1H), 2.04 (dd, J=2.0, 13.2 Hz, 2H), 1.85 (d, J=10.8 Hz, 2H), 1.53-1.41 (m, 3H), 1.11-1.01 (m, 2H); LC-MS (ESI⁺) m/z 419.1 (M+H)⁺.

### Step 9—N-[2-(4-formylcyclohexyl)pyrazolo[1,5-a]pyridin-5-yl]-6-(trifluoromethyl)pyridine-2-carboxamide

To a solution of N-[2-[4-(hydroxymethyl)cyclohexyl]pyrazolo[1,5-a]pyridin-5-yl]-6-(trifluoromethyl) pyridine-2-carboxamide (26 mg, 62.1 umol) in DCM (1 mL) was added DMP (31.6 mg, 74.5 umol). The reaction mixture was stirred at 25° C. for 2 hrs. On completion, the reaction mixture was quenched by saturated Na₂S₂O₃ (2 mL) and saturated NaHCO₃ (2 mL) at 25° C., and then stirred for 15 minutes. The mixture was extracted with DCM (2×5 mL), then the combined organic layers was washed with saturated NaCl (2×10 mL). Then the combined organic layers was dried over Na₂SO₄, filtered and concentrated in vacuo to give the title compound (24 mg, 63% yield) as a brown solid. LC-MS (ESI⁺) m/z 417.1 (M+H)⁺.

### 3-[4-(2,6-Diazaspiro[3.3]heptan-2-ylmethyl)-3-methyl-2-oxo-benzimidazol-1-yl]piperidine-2,6-dione (Intermediate CAJ)

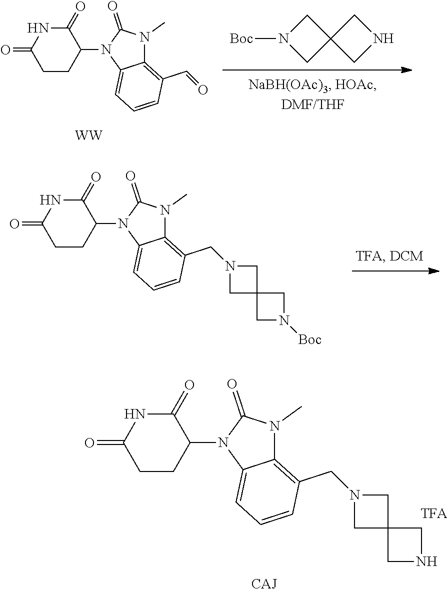 [SMILES: Cn1c(=O)n(C2CCC(=O)NC2=O)c2cccc(C=O)c21 ; CC(C)(C)OC(=O)N1CC2(CNC2)C1 ; "WW" ; Cn1c(=O)n(C2CCC(=O)NC2=O)c2cccc(CN3CC4(C3)CN(C(=O)OC(C)(C)C)C4)c21 ; Cn1c(=O)n(C2CCC(=O)NC2=O)c2cccc(CN3CC4(CNC4)C3)c21]

### Step 1—Tert-butyl 6-[[1-(2,6-dioxo-3-piperidyl)-3-methyl-2-oxo-benzimidazol-4-yl]methyl]-2,6-diazaspiro[3.3]heptane-2-carboxylate

To a solution of tert-butyl 2,6-diazaspiro[3.3]heptane-2-carboxylate; oxalic acid (100 mg, 348 umol, CAS #1041026-70-3) in DMF (0.5 mL) was added TEA (70.4 mg, 696 umol). The reaction mixture was stirred at 25° C. for 10 min. Then HOAc (41.8 mg, 696 umol) and 1-(2,6-dioxo-3-piperidyl)-3-methyl-2-oxo-benzimidazole-4-carbaldehyde (100 mg, 348 umol, Intermediate WW) was added at 25° C. The mixture was stirred at 25° C. for 20 min. Then NaBH(OAc)₃ (88.5 mg, 417 umol) was added to the mixture and the mixture was stirred at 25° C. for 2 hrs. On completion, the reaction mixture was quenched with water (0.05 mL) and concentrated in vacuo to give a residue. The residue was purified by reverse phase (0.1% FA condition) to give the title compound (100 mg, 56% yield) as a yellow solid. ¹H NMR (400 MHz, DMSO-d₆) δ 11.09 (s, 1H), 7.05 (d, J=7.6 Hz, 1H), 6.99-6.85 (m, 2H), 5.36 (dd, J=5.6, 12.4 Hz, 1H), 4.23 (s, 1H), 3.98 (s, 3H), 3.89 (s, 4H), 3.76 (s, 2H), 3.62 (s, 3H), 2.95-2.82 (m, 1H), 2.77-2.61 (m, 2H), 2.04-1.96 (m, 1H), 1.36 (d, J=6.0 Hz, 9H); LC-MS (ESI⁺) m/z 470.0 (M+H)⁺.

### Step 2—3-[4-(2,6-Diazaspiro[3.3]heptan-2-ylmethyl)-3-methyl-2-oxo-benzimidazol-1-yl]piperidine-2,6-dione

To a solution of tert-butyl 6-[[1-(2,6-dioxo-3-piperidyl)-3-methyl-2-oxo-benzimidazol-4-yl]methyl]-2,6-diazaspiro[3.3]heptane-2-carboxylate (50 mg, 106 umol) in DCM (1 mL) was added TFA (308 mg, 2.70 mmol). The mixture was then stirred at 25° C. for 0.5 hr. On completion, the reaction mixture was concentrated in vacuo to give title compound (50 mg, 97% yield, TFA salt) as yellow oil. LC-MS (ESI⁺) m/z 370.1 (M+H)⁺.

### 3-[5-methoxy-3-methyl-2-oxo-4-(4-piperidyl)benzimidazol-1-yl]-1-methyl-piperidine-2,6-dione (Intermediate CAK)

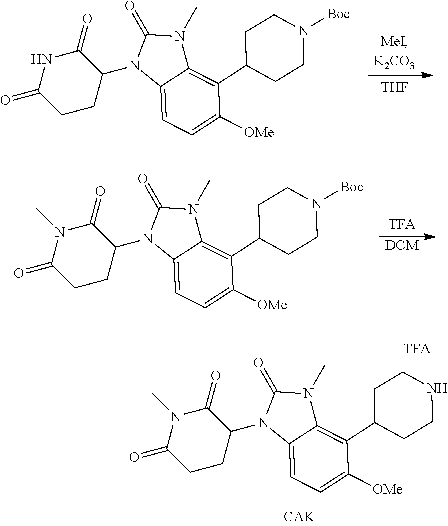

### Step 1—Tert-butyl 4-[5-methoxy-3-methyl-1-(1-methyl-2,6-dioxo-3-piperidyl)-2-oxo-benzimidazol-4-yl]piperidine-1-carboxylate

A mixture of tert-butyl 4-[1-(2,6-dioxo-3-piperidyl)-5-methoxy-3-methyl-2-oxo-benzimidazol-4-yl]piperidine-1-carboxylate (100 mg, 211 umol, synthesized via Steps 1-5 of Intermediate BUC), MeI (45.0 mg, 317 umol) and K₂CO₃ (87.7 mg, 634 umol) in THF (1 mL) was stirred at 50° C. for 1 hr. On completion, the reaction was diluted with EA (100 mL). The organic layer was washed with water (100 mL×3), dried over Na₂SO₄ and filtered. The filtrate was concentrated in vacuo to give title compound (100 mg, 97% yield) as yellow solid. ¹H NMR (400 MHz, DMSO-d₆) δ 7.43 (d, J=8.8 Hz, 1H), 7.21 (d, J=8.8 Hz, 1H), 5.90 (dd, J=5.6, 12.8 Hz, 1H), 4.54 (q, J=7.2 Hz, 2H), 4.24 (s, 3H), 4.11-4.07 (m, 3H), 4.04-3.92 (m, 1H), 3.54 (s, 3H), 3.50-3.41 (m, 1H), 3.38-3.24 (m, 3H), 3.22-3.14 (m, 1H), 2.79-2.67 (m, 2H), 2.09 (d, J=11.2 Hz, 2H), 1.95 (s, 9H), 1.69 (t, J=7.2 Hz, 1H).

### Step 2—3-[5-methoxy-3-methyl-2-oxo-4-(4-piperidyl)benzimidazol-1-yl]-1-methyl-piperidine-2,6-dione

A mixture of tert-butyl 4-[5-methoxy-3-methyl-1-(1-methyl-2,6-dioxo-3-piperidyl)-2-oxo-benzimidazol-4-yl]piperidine-1-carboxylate (40.0 mg, 82.2 umol) and TFA (385 mg, 3.38 mmol) in DCM (1 mL) was stirred at 25° C. for 1 hr. On completion, the reaction was concentrated in vacuo to give title compound (41 mg, 99% yield, TFA) as brown oil. LC-MS (ESI⁺) m/z 387.4 (M+H)⁺.

### [3-Methyl-4-[(3S)-3-methylpiperazin-1-yl]-2-oxo-benzimidazol-1-yl]piperidine-2,6-dione (Intermediate CAL)

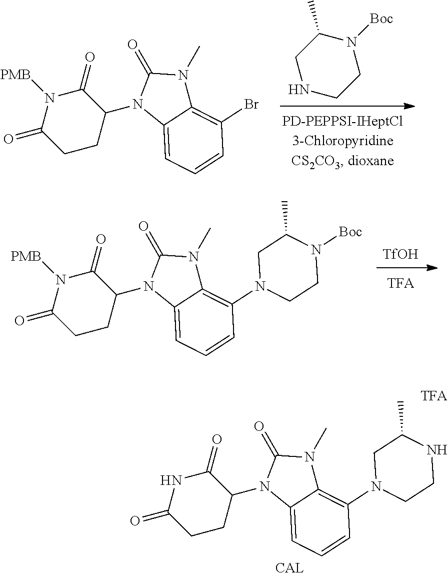

### Step 1—Tert-butyl (2S)-4-[1-[1-[(4-methoxyphenyl)methyl]-2,6-dioxo-3-piperidyl]-3-methyl-2-oxo-benzimidazol-4-yl]-2-methyl-piperazine-1-carboxylate

To a solution of 3-(4-bromo-3-methyl-2-oxo-benzimidazol-1-yl)-1-[(4-methoxyphenyl)methyl]piperidine-2,6-dione (800 mg, 1.75 mmol, synthesized via Steps 1-4 of Intermediate HP) and tert-butyl (2S)-2-methylpiperazine-1-carboxylate (419 mg, 2.09 mmol, CAS #169447-70-5) in dioxane (15 mL) was added PD-PEPPSI-IHeptCl 3-Chloropyridine (100 mg, 103 umol) and Cs₂CO₃ (1.14 g, 3.49 mmol). Then the mixture was stirred at 100° C. for 16 hrs. On completion, the mixture was diluted with H₂O (30 mL) and extracted with EA (30 mL×3). The combined organic layers were washed with saturated NaCl (20 mL), dried over Na₂SO₄, filtered and concentrated in vacuo to give a residue. The residue was triturated with EA:PE=1:20 and filtered to give the title compound (450 mg, 45% yield) as yellow solid. ¹H NMR (400 MHz, DMSO-d₆) δ 7.22 (d, J=8.4 Hz, 2H), 7.12-6.98 (m, 1H), 6.94 (s, 1H), 6.90-6.80 (m, 3H), 5.57-5.47 (m, 1H), 4.85-4.74 (m, 2H), 4.27-4.25 (m, 1H), 3.73 (s, 2H), 3.82-3.72 (m, 1H), 3.68 (s, 3H), 3.15-3.00 (m, 3H), 2.92-2.69 (m, 3H), 2.09-2.01 (m, 1H), 1.44 (s, 9H), 1.28-1.27 (m, 3H), 1.18-1.05 (m, 1H), 0.87-0.81 (m, 2H).

### Step 2—[3-Methyl-4-[(3S)-3-methylpiperazin-1-yl]-2-oxo-benzimidazol-1-yl]piperidine-2,6-dione

A solution of tert-butyl (2S)-4-[1-[1-[(4-methoxyphenyl)methyl]-2,6-dioxo-3-piperidyl]-3-methyl-2-oxo-benzimidazol-4-yl]-2-methyl-piperazine-1-carboxylate (200 mg, 346 umol) in TFA (1.5 mL) was added TfOH (0.3 mL), then the mixture was stirred at 70° C. for 1 hr. On completion, the mixture was concentrated in vacuo to give the title compound (150 mg, 92% yield, TFA) as brown oil. LC-MS (ESI⁺) m/z 358.2 (M+H)⁺.

### 5-chlorothiazole-2-carboxylic acid (CAS #101012-16-2) (Intermediate CAM)

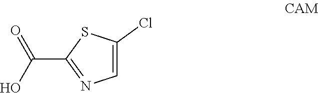

### 3-[4-(8-azabicyclo[3.2.1]octan-3-yl)-3-methyl-2-oxo-benzimidazol-1-yl]piperidine-2,6-dione (Intermediate CAN)

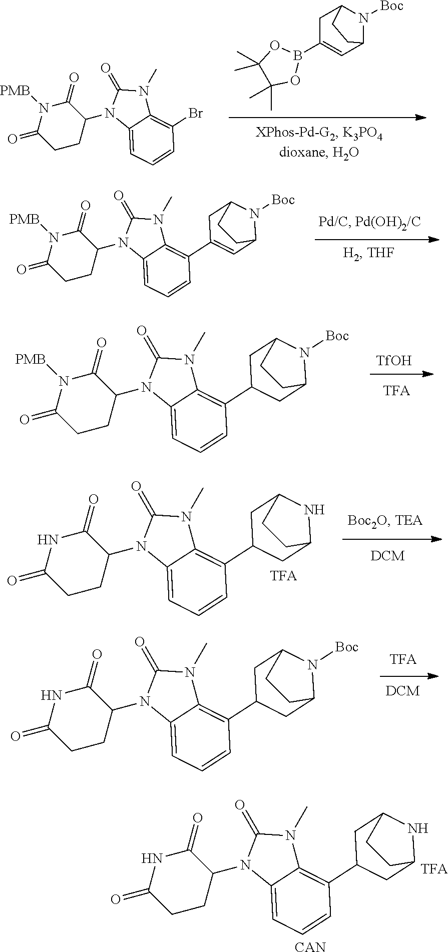

### Step 1—3-[1-[1-[(4-methoxyphenyl)methyl]-2,6-dioxo-3-piperidyl]-3-methyl-2-oxo-benzimidazol-4-yl]-8-azabicyclo[3.2.1]oct-2-ene-8-carboxylate

To a solution of 3-(4-bromo-3-methyl-2-oxo-benzimidazol-1-yl)-1-[(4-methoxyphenyl)methyl]piperidine-2,6-dione (500 mg, 1.09 mmol, synthesized via Steps 1-4 of Intermediate HP), tert-butyl 3-(4,4,5,5-tetramethyl-1,3,2-dioxaborolan-2-yl)-8-azabicyclo[3.2.1]oct-2-ene-8-carboxylate (365 mg, 1.09 mmol, CAS #900503-08-4) and K₃PO₄ (694 mg, 3.27 mmol) in dioxane (10 mL) and H₂O (2 mL) was added XPHOS-PD-G₂ (85.8 mg, 109 umol). The reaction mixture was stirred at 80° C. under N₂ for 2 hrs. On completion, the reaction mixture was filtered and the filtrate was concentrated in vacuo. The residue was purified by column chromatography (SiO₂, PE/EA=3/1 to 1/1; PE/EA=1/1, P1:R_{f}=0.3) to give title compound (589 mg, 92% yield) as white solid. ¹H NMR (400 MHz, DMSO-d₆) δ 7.21 (d, J=8.4 Hz, 2H), 6.99-6.89 (m, 2H), 6.85 (d, J=8.8 Hz, 2H), 6.80-6.72 (m, 1H), 6.06-5.99 (m, 1H), 5.57-5.49 (m, 1H), 4.79 (q, J=14.4 Hz, 2H), 4.37 (s, 1H), 4.32-4.21 (m, 1H), 3.72 (s, 3H), 3.30 (s, 3H), 3.10-2.99 (m, 1H), 2.96-2.69 (m, 3H), 2.25-2.12 (m, 2H), 2.08-2.01 (m, 1H), 1.96 (s, 2H), 1.89-1.78 (m, 1H), 1.42 (s, 9H); LC-MS (ESI⁺) m/z 587.3 (M+H)⁺.

### Step 2—Tert-butyl3-[1-[1-[(4-methoxyphenyl)methyl]-2,6-dioxo-3-piperidyl]-3-methyl-2-oxo-benzimidazol-4-yl]-8-azabicyclo[3.2.1]octane-8-carboxylate

To a solution of tert-butyl 3-[1-[1-[(4-methoxyphenyl)methyl]-2,6-dioxo-3-piperidyl]-3-methyl-2-oxo-benzimidazol-4-yl]-8-azabicyclo[3.2.1]oct-2-ene-8-carboxylate (500 mg, 852 umol) in THF (5 mL) was added Pd/C (200 mg, 852 umol, 10 wt %) and Pd/(OH)₂/C (200 mg, 852 umol, 10 wt %) under N₂. The suspension was degassed under vacuo and purged with H₂ several times. The mixture was stirred under H₂ (15 psi) at 25° C. for 4 hrs. On completion, the reaction was filtered and concentrated in vacuo to give title compound (500 mg, 99% yield) as yellow solid. ¹H NMR (400 MHz, DMSO-d₆) δ 7.20 (d, J=8.8 Hz, 2H), 7.06 (d, J=7.6 Hz, 1H), 7.01-6.92 (m, 1H), 6.90-6.77 (m, 3H), 5.57-5.46 (m, 1H), 4.79 (q, J=14.4 Hz, 2H), 4.17 (s, 2H), 3.72 (s, 3H), 3.66 (s, 1H), 3.62-3.58 (m, 1H), 3.51 (s, 2H), 3.23-3.11 (m, 1H), 3.10-2.96 (m, 1H), 2.85-2.64 (m, 2H), 2.44-2.31 (m, 2H), 2.07-1.90 (m, 3H), 1.78-1.73 (m, 2H), 1.47-1.41 (m, 9H), 1.35 (s, 1H).

### Step 3—3-[4-(8-azabicyclo[3.2.1]octan-3-yl)-3-methyl-2-oxo-benzimidazol-1-yl]piperidine-2,6-dione

To a solution of tert-butyl 3-[1-[1-[(4-methoxyphenyl)methyl]-2,6-dioxo-3-piperidyl]-3-methyl-2-oxo-benzimidazol-4-yl]-8-azabicyclo[3.2.1]octane-8-carboxylate (250 mg, 424 umol) in TFA (7.70 g, 67.5 mmol, 5 mL) was added TfOH (2.13 g, 14.1 mmol, 1.25 mL). The reaction mixture was stirred at 70° C. for 2 hrs. On completion, the reaction mixture was concentrated in vacuo to give title compound (156 mg, 76% yield, TFA) as brown oil. LC-MS (ESI⁺) m/z 369.2 (M+H)⁺.

### Step 4—Tert-butyl 3-[1-(2,6-dioxo-3-piperidyl)-3-methyl-2-oxo-benzimidazol-4-yl]-8-azabicyclo[3.2.1]octane-8-carboxylate

To a solution of 3-[4-(8-azabicyclo[3.2.1]octan-3-yl)-3-methyl-2-oxo-benzimidazol-1-yl]piperidine-2,6-dione (156 mg, 323 umol, TFA) and TEA (327 mg, 3.23 mmol, 450 uL) in DCM (2 mL) was added Boc₂O (106 mg, 485 umol, 111 uL). The reaction mixture was stirred at 25° C. for 2 hrs. On completion, the reaction mixture was concentrated in vacuo. The residue was purified by prep-HPLC (column: Phenomenex luna C18 150*25 mm*10 um; mobile phase: [water(0.225% FA)-ACN]; B %: 38%-68%, 10 min) to give title compound (25 mg, 16% yield) as white solid. ¹H NMR (400 MHz, DMSO-d₆) δ 11.09 (s, 1H), 7.11-6.99 (m, 2H), 6.98-6.94 (m, 1H), 5.40-5.33 (m, 1H), 4.17 (s, 2H), 3.66 (s, 1H), 3.51 (s, 3H), 2.90-2.83 (m, 1H), 2.76-2.68 (m, 1H), 2.65-2.58 (m, 1H), 2.05-1.93 (m, 3H), 1.88-1.82 (m, 1H), 1.78-1.67 (m, 2H), 1.60-1.50 (m, 1H), 1.47-1.41 (m, 11H).

### Step 5—3-[4-(8-azabicyclo[3.2.1]octan-3-yl)-3-methyl-2-oxo-benzimidazol-1-yl]piperidine-2,6-dione

To a solution of tert-butyl 3-[1-(2,6-dioxo-3-piperidyl)-3-methyl-2-oxo-benzimidazol-4-yl]-8-azabicyclo[3.2.1]octane-8-carboxylate (42.8 mg, 91.3 umol) in DCM (1 mL) was added TFA (459 mg, 4.03 mmol, 298 uL). The reaction was stirred at 25° C. for 2 hrs. On completion, the reaction mixture was concentrated in vacuo to give title compound (44 mg, 99% yield, TFA) as brown oil. LC-MS (ESI⁺) m/z 369.2 (M+H)⁺.

### 1-[8-[(1S,4S)-2,5-diazabicyclo[2.2.1]heptan-2-yl]-4-isoquinolyl]hexahydropyrimidine-2,4-dione (Intermediate CAO)

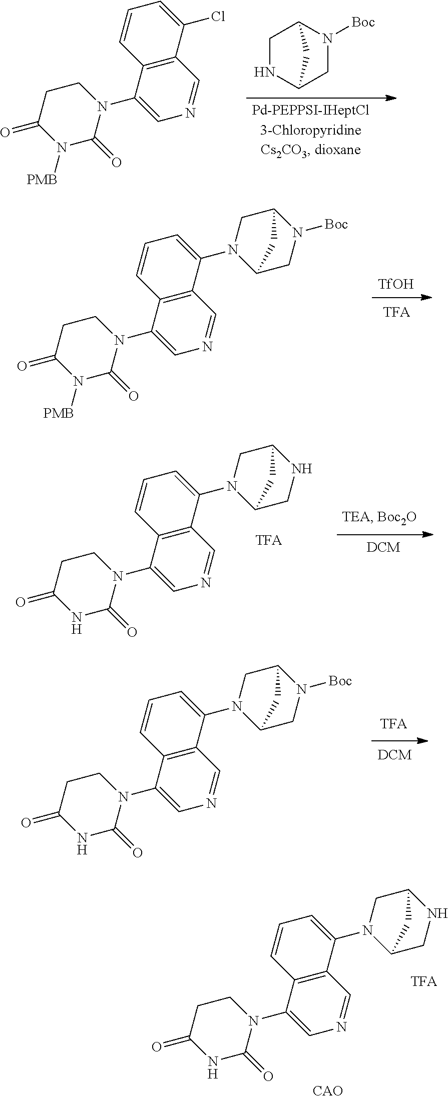

### Step 1—Tert-butyl (1S,4S)-5-[4-[3-[(4-methoxyphenyl)methyl]-2,4-dioxo-hexahydropyrimidin-1-yl]-8-isoquinolyl]-2,5-diazabicyclo[2.2.1]heptane-2-carboxylate

A solution of 1-(8-chloro-4-isoquinolyl)-3-[(4-methoxyphenyl)methyl]hexahydropyrimidine-2,4-dione (250 mg, 631 umol, synthesized via Steps 1-2 of Intermediate BSL), tert-butyl (1S,4S)-2,5-diazabicyclo[2.2.1]heptane-2-carboxylate (137 mg, 694 umol, CAS #113451-59-5), Cs₂CO₃ (411 mg, 1.26 mmol) and PD-PEPPSI-IHeptCl₃-Chloropyridine (30.7 mg, 31.5 umol) in dioxane (4.5 mL) was stirred at 80° C. under N₂ for 16 hrs. On completion, the reaction mixture was filtered and the filtrate was concentrated in vacuo. The residue was purified by reversed phase HPLC (0.10% FA condition) to give the title compound (330 mg, 93% yield) as yellow solid. ¹H NMR (400 MHz, DMSO-d₆) δ 9.38 (d, J=8.0 Hz, 1H), 8.45 (d, J=6.8 Hz, 1H), 7.62-7.54 (m, 1H), 7.31-7.23 (m, 3H), 7.03 (t, J=8.4 Hz, 1H), 6.91-6.85 (m, 2H), 4.86-4.81 (m, 2H), 4.59-4.52 (m, 1H), 4.50-4.42 (m, 1H), 4.14-4.01 (m, 1H), 3.94-3.79 (m, 1H), 3.73 (s, 3H), 3.63-3.54 (m, 1H), 3.16-3.05 (m, 1H), 2.99-2.91 (m, 1H), 2.58-2.53 (m, 1H), 2.08-1.89 (m, 2H), 1.48-1.25 (m, 11H); LC-MS (ESI⁺) m/z 558.3 (M+H)⁺.

### Step 2—1-[8-[(1S,4S)-2,5-diazabicyclo[2.2.1]heptan-2-yl]-4-isoquinolyl]hexahydropyrimidine-2,4-dione

To a solution of tert-butyl (1S,4S)-5-[4-[3-[(4-methoxyphenyl)methyl]-2,4-dioxo-hexahydropyrimidin-1-yl]-8-isoquinolyl]-2,5-diazabicyclo[2.2.1]heptane-2-carboxylate (150 mg, 268 umol) in TFA (3.08 g, 27.0 mmol) was added TfOH (680 mg, 4.53 mmol). The reaction mixture was then stirred at 70° C. for 2 hrs. On completion, the reaction mixture was concentrated in vacuo to give the title compound (121 mg, 99% yield, TFA) as red oil. LC-MS (ESI⁺) m/z 338.2 (M+H)⁺.

### Step 3—Tert-butyl (1S,4S)-5-[4-(2,4-dioxohexahydropyrimidin-1-yl)-8-isoquinolyl]-2,5-diazabicyclo [2.2.1]heptane-2-carboxylate

To a solution of 1-[8-[(1S,4S)-2,5-diazabicyclo[2.2.1]heptan-2-yl]-4-isoquinolyl]hexahydropyrimidine-2,4-dione (120 mg, 265 umol, TFA) and TEA (269 mg, 2.66 mmol) in DCM (1.5 mL) was added Boc₂O (87.0 mg, 398 umol) at 25° C. The reaction mixture was then stirred at 25° C. for 2 hrs. On completion, the reaction mixture was diluted with DCM (30 mL) and washed with water (30 mL×3). The organic layer was dried over Na₂SO₄, filtered and concentrated in vacuo to give the title compound (114 mg, 98% yield) as yellow solid. LC-MS (ESI⁺) m/z 438.2 (M+H)⁺.

### Step 4—1-[8-[(1S,4S)-2,5-diazabicyclo[2.2.1]heptan-2-yl]-4-isoquinolyl]hexahydropyrimidine-2,4-dione

To a solution of tert-butyl (1S,4S)-5-[4-(2,4-dioxohexahydropyrimidin-1-yl)-8-isoquinolyl]-2,5-diazabicyclo[2.2.1]heptane-2-carboxylate (95.0 mg, 217 umol) in DCM (1 mL) was added TFA (462 mg, 4.05 mmol). The reaction mixture was then stirred at 25° C. for 1 hr. On completion, the reaction mixture was concentrated in vacuo to give the title compound (98.0 mg, 99% yield, TFA) as red oil. LC-MS (ESI⁺) m/z 338.2 (M+H)⁺.

### 3-(4-bromo-3-ethyl-2-oxo-benzimidazol-1-yl)-1-[(4-methoxyphenyl)methyl]piperidine-2,6-dione (Intermediate CAP)

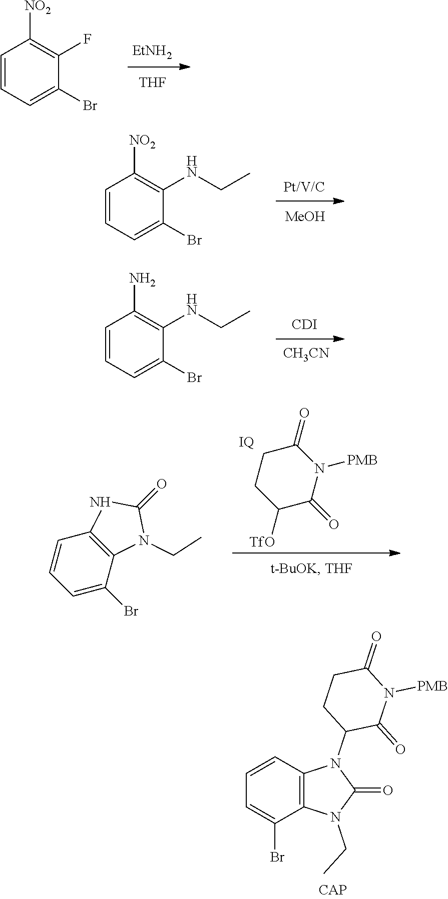

### Step 1—2-Bromo-N-ethyl-6-nitro-aniline

To a solution of 1-bromo-2-fluoro-3-nitro-benzene (5 g, 22.7 mmol, CAS #58534-94-4) in THF (50 mL) was added ethanamine (2.05 g, 45.4 mmol) at 0° C. under N₂. The reaction was stirred at 25° C. for 16 hrs. On completion, the mixture was quenched with H₂O (50 mL), diluted with EA (50 mL), and extracted with EA (3×50 mL). The combined organic layer was washed with brine (3×50 mL), filtered and concentrated in vacuo to give the title compound (5.5 g, 100% yield) as yellow solid. ¹H NMR (400 MHz, DMSO-d₆) δ 7.86-7.78 (m, 2H), 6.80 (t, J=8.0 Hz, 1H), 5.99-5.89 (m, 1H), 3.11 (q, J=7.2 Hz, 2H), 1.11 (t, J=7.2 Hz, 3H). LC-MS (ESI⁺) m/z 244.8. (M+H)⁺.

### Step 2—3-Bromo-N2-ethyl-benzene-1,2-diamine

To a solution of 2-bromo-N-ethyl-6-nitro-aniline (500 mg, 2.04 mmol) in THF (8 mL) was added Pt/V/C (250 mg, 957 umol) under N₂. The suspension was degassed under vacuum and purged with H₂ three times. The mixture was stirred under H₂ (15 psi) at 25° C. for 16 hours. On completion, the mixture was filtered and concentrated in vacuo to give the title compound (400 mg, 91% yield) as brown oil. ¹H NMR (400 MHz, DMSO-d₆) δ 6.74-6.69 (m, 1H), 6.68-6.59 (m, 2H), 5.36-4.63 (m, 2H), 2.90 (q, J=7.2 Hz, 2H), 1.09 (t, J=7.2 Hz, 3H). LC-MS (ESI⁺) m/z 217.0 (M+H)⁺.

### Step 3—4-Bromo-3-ethyl-1H-benzimidazol-2-one

To a solution of 3-bromo-N2-ethyl-benzene-1,2-diamine (400 mg, 1.86 mmol) in ACN (10 mL) was added CDI (361 mg, 2.23 mmol). The mixture was stirred at 80° C. for 16 hrs. On completion, the mixture was dropwise into H₂O (20 mL), then filtered and concentrated in vacuo to give the title compound (270 mg, 60% yield) as yellow solid. ¹H NMR (400 MHz, DMSO-d₆) δ 11.19 (s, 1H), 7.17 (dd, J=1.2, 8.0 Hz, 1H), 7.01-6.97 (m, 1H), 6.95-6.89 (m, 1H), 4.11 (q, J=7.2 Hz, 2H), 1.22 (t, J=7.2 Hz, 3H). LC-MS (ESI⁺) m/z 240.8 (M+H)⁺.

### Step 4—3-(4-Bromo-3-ethyl-2-oxo-benzimidazol-1-yl)-1-[(4-methoxyphenyl)methyl]piperidine-2,6-dione

To a solution of 4-bromo-3-ethyl-1H-benzimidazol-2-one (240 mg, 995 umol) in THF (4 mL) was stirred at −10° C., then the t-BuOK (201 mg, 1.79 mmol) was added in above solution was stirred at −10° C. for 1.5 hrs. Then a solution of [1-[(4-methoxyphenyl)methyl]-2,6-dioxo-3-piperidyl]trifluoromethanesulfonate (379 mg, 995 umol, Intermediate IQ) in THF (4 mL) was dropwise added into above solution. The mixture was stirred at −10° C. for 2.5 hrs. On completion, the mixture was quenched by NH₄Cl (10 mL), diluted with H₂O (20 mL), and extracted with EA (2×20 mL). Then the combined organic layer was washed with brine (2×20 mL), dried with anhydrous Na₂SO₄ and concentrated in vacuo. The mixture was purified by reversed phase (0.1% FA) to give the title compound (340 mg, 72% yield) as green solid. ¹H NMR (400 MHz, DMSO-d₆) δ 7.28-7.16 (m, 3H), 7.08 (d, J=6.8 Hz, 1H), 6.99-6.91 (m, 1H), 6.89-6.81 (m, 2H), 5.57 (dd, J=5.6, 13.2 Hz, 1H), 4.79 (q, J=14.4 Hz, 2H), 4.25-4.14 (m, 2H), 3.72 (s, 3H), 3.12-2.98 (m, 1H), 2.89-2.73 (m, 2H), 2.12-2.04 (m, 1H), 1.26 (t, J=7.2 Hz, 3H). LC-MS (ESI⁺) m/z 474.1 (M+H)⁺.

### 3-(3-ethyl-2-oxo-4-piperazin-1-yl-benzimidazol-1-yl)piperidine-2,6-dione (Intermediate CAQ)

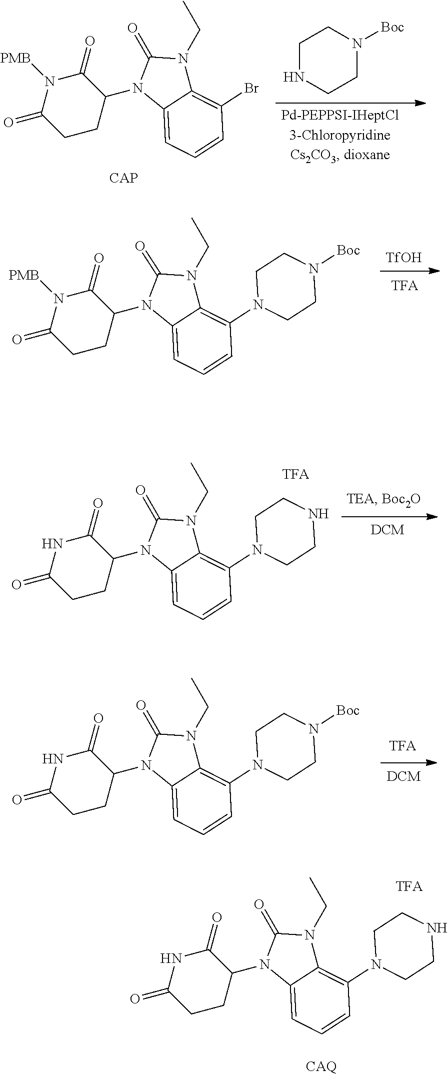

### Step 1—Tert-butyl4-[3-ethyl-1-[1-[(4-methoxyphenyl)methyl]-2,6-dioxo-3-piperidyl]-2-oxo-benzimidazol-4-yl]piperazine-1-carboxylate

A mixture of 3-(4-bromo-3-ethyl-2-oxo-benzimidazol-1-yl)-1-[(4-methoxyphenyl)methyl]piperidine-2,6-dione (200 mg, 423 umol, Intermediate CAP), tert-butyl piperazine-1-carboxylate (157 mg, 846 umol, CAS #143238-38-4), Cs₂CO₃ (413 mg, 1.27 mmol) and Pd-PEPPSI-IHeptCl₃-Chloropyridine (41.1 mg, 42.3 umol) in dioxane (3.5 mL) was stirred at 100° C. for 5 hrs under N₂. On completion, the reaction mixture was diluted with EtOAc (30 mL), filtered and concentrated in vacuo. The residue was purified by prep-HPLC (column: Phenomenex luna C18 150*25 mm*10 um; mobile phase: [water(0.225% FA)-ACN]; B %: 50%-80%, 10 min) to give the title compound (149 mg, 60% yield) as yellow solid. ¹H NMR (400 MHz, DMSO-d₆) δ 7.21 (d, J=8.4 Hz, 2H), 7.02-6.91 (m, 2H), 6.88-6.78 (m, 3H), 5.54-5.48 (m, 1H), 4.79 (q, J=14.4 Hz, 2H), 4.11 (q, J=6.8 Hz, 2H), 4.02-3.92 (m, 2H), 3.72 (s, 3H), 3.11-2.92 (m, 5H), 2.84-2.68 (m, 4H), 2.08-2.01 (m, 1H), 1.43 (s, 9H), 1.22-1.17 (m, 3H); LC-MS (ESI⁺) m/z 578.3 (M+H)⁺.

### Step 2—3-(3-ethyl-2-oxo-4-piperazin-1-yl-benzimidazol-1-yl)piperidine-2,6-dione

To a solution of tert-butyl 4-[3-ethyl-1-[1-[(4-methoxyphenyl)methyl]-2,6-dioxo-3-piperidyl]-2-oxo-benzimidazol-4-yl]piperazine-1-carboxylate (140 mg, 242 umol) in TFA (1.5 mL) was added TfOH (510 mg, 3.40 mmol). The reaction mixture was then stirred at 70° C. for 2 hrs. On completion, the reaction mixture was concentrated in vacuo to give title compound (114 mg, 99% yield, TFA) as red oil. LC-MS (ESI⁺) m/z 358.2 (M+H)⁺.

### Step 3—Tert-butyl 4-[1-(2,6-dioxo-3-piperidyl)-3-ethyl-2-oxo-benzimidazol-4-yl]piperazine-1-carboxylate

To a solution of 3-(3-ethyl-2-oxo-4-piperazin-1-yl-benzimidazol-1-yl)piperidine-2,6-dione (110 mg, 233 umol, TFA) and TEA (47.2 mg, 466 umol) in DCM (1 mL) was added Boc₂O (76.3 mg, 350 umol) at 25° C. The reaction mixture was then stirred at 25° C. for 16 hrs. On completion, the reaction mixture was diluted with DCM (30 mL) and washed with water (20 mL×3). The organic layer was dried over Na₂SO₄, filtered and concentrated in vacuo. The residue was purified by column chromatography (SiO₂, PE/EA=0/1) to give the title compound (100 mg, 93% yield) as yellow solid. 1H NMR (400 MHz, DMSO-d₆) δ 11.08 (s, 1H), 7.01 (d, J=4.4 Hz, 2H), 6.94 (q, J=4.4 Hz, 1H), 5.38-5.31 (m, 1H), 4.11 (q, J=6.8 Hz, 2H), 4.01-3.93 (m, 2H), 2.98 (d, J=10.4 Hz, 3H), 2.95-2.82 (m, 2H), 2.80-2.73 (m, 2H), 2.70-2.58 (m, 2H), 2.07-1.99 (m, 1H), 1.43 (s, 9H), 1.22-1.19 (m, 3H); LC-MS (ESI+) m/z 458.2 (M+H)⁺.

### Step 4—3-(3-ethyl-2-oxo-4-piperazin-1-yl-benzimidazol-1-yl)piperidine-2,6-dione

To a solution of tert-butyl 4-[1-(2,6-dioxo-3-piperidyl)-3-ethyl-2-oxo-benzimidazol-4-yl]piperazine-1-carboxylate (45.0 mg, 98.3 umol) in DCM (0.5 mL) was added TFA (770 mg, 6.75 mmol) at 25° C. The reaction mixture was then stirred at 25° C. for 2 hrs. On completion, the reaction mixture was concentrated in vacuo to give the title compound (46.3 mg, 100% yield, TFA) was obtained as brown solid. LC-MS (ESI⁺) m/z 358.2 (M+H)⁺.

### 3-[4-(1,4-Diazepan-1-yl)-3-methyl-2-oxo-benzimidazol-1-yl]piperidine-2,6-dione (Intermediate CAR)

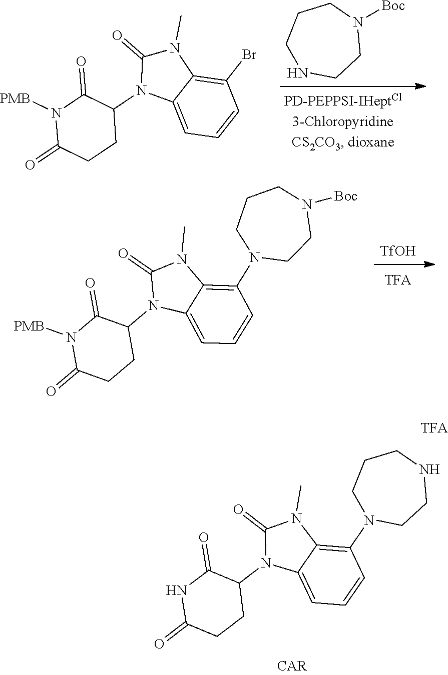

### Step 1—Tert-butyl 4-[1-[1-[(4-methoxyphenyl)methyl]-2,6-dioxo-3-piperidyl]-3-methyl-2-oxo-benzimidazol-4-yl]-1,4-diazepane-1-carboxylate

To a solution of 3-(4-bromo-3-methyl-2-oxo-benzimidazol-1-yl)-1-[(4-methoxyphenyl)methyl]piperidine-2,6-dione (300 mg, 654 umol, synthesized via Steps 1-4 of Intermediate HP) and tert-butyl 1,4-diazepane-1-carboxylate (131 mg, 654 umol, CAS #112275-50-0) in dioxane (5 mL) was added Cs₂CO₃ (426 mg, 1.31 mmol) and Pd-PEPPSI-IHept^{Cl} 3-Chloropyridine (51.9 mg, 65.4 umol). The mixture was stirred at 80° C. for 16 hrs. On completion, the reaction mixture was filtered and the filtrate was concentrated in vacuo to give a residue. The residue was purified by column chromatography (SiO₂, PE:EA=5:1 to PE:EA=1:1, PE:EA=3:1, P1:Rf=0.3) to give the title compound (217 mg, 57% yield) as an off-white solid. 1H NMR (400 MHz, DMSO-d₆) δ 7.21 (d, J=8.8 Hz, 2H), 6.93 (d, J=4.0 Hz, 2H), 6.85 (d, J=8.8 Hz, 2H), 6.78 (s, 1H), 5.58-5.43 (m, 1H), 4.84-4.73 (m, 2H), 3.72 (s, 3H), 3.60 (d, J=4.4 Hz, 3H), 3.56-3.41 (m, 4H), 3.08 (d, J=6.0 Hz, 4H), 2.87-2.63 (m, 3H), 2.10-2.00 (m, 1H), 1.86 (s, 2H), 1.43 (d, J=4.4 Hz, 9H); LC-MS (ESI+) m/z 578.4 (M+H)⁺.

### Step 2—3-[4-(1,4-Diazepan-1-yl)-3-methyl-2-oxo-benzimidazol-1-yl]piperidine-2,6-dione

To a solution of tert-butyl 4-[1-[1-[(4-methoxyphenyl)methyl]-2,6-dioxo-3-piperidyl]-3-methyl-2-oxo-benzimidazol-4-yl]-1,4-diazepane-1-carboxylate (100 mg, 173 umol) in TFA (2 mL) was added TfOH (510 mg, 3.40 mmol, 0.3 mL). Then the reaction mixture was stirred at 70° C. for 2 hrs. On completion, the reaction mixture was concentrated in vacuo to give the title compound (65 mg, 90% yield, TFA salt) as brown oil. LC-MS (ESI+) m/z 358.2 (M+H)⁺.

### 1-[8-[(2R)-2-methylpiperazin-1-yl]-4-isoquinolyl]hexahydropyrimidine-2,4-dione (Intermediate CAS)

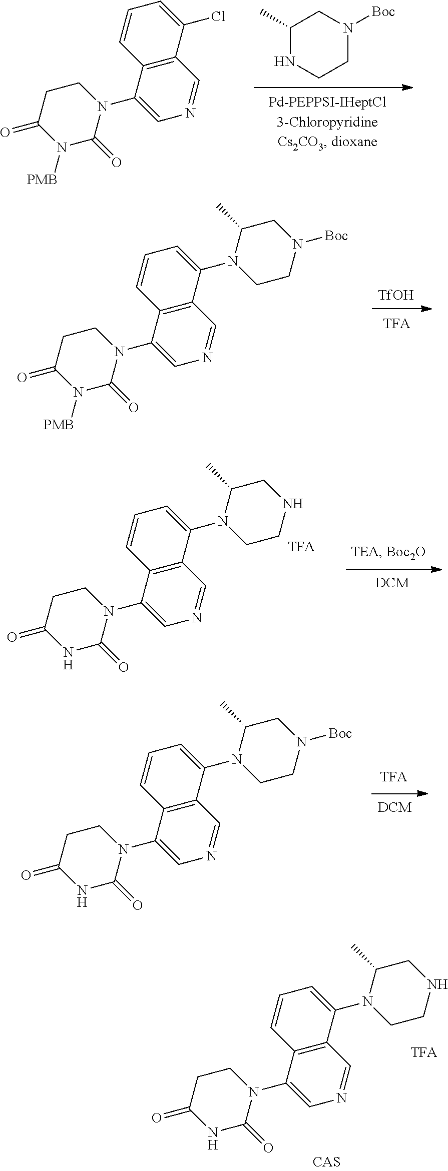

### Step 1—Tert-butyl(3R)-4-[4-[3-[(4-methoxyphenyl)methyl]-2,4-dioxo-hexahydropyrimidin-1-yl]-8-isoquinolyl]-3-methyl-piperazine-1-carboxylate

A mixture of 1-(8-chloro-4-isoquinolyl)-3-[(4-methoxyphenyl)methyl]hexahydropyrimidine-2,4-dione (250 mg, 631 umol, synthesized via Steps 1-2 of Intermediate BSL), tert-butyl (3R)-3-methylpiperazine-1-carboxylate (189 mg, 947 umol, CAS #163765-44-4), Cs₂CO₃ (617 mg, 1.89 mmol) and PD-PEPPSI-IHeptCl₃-Chloropyridine (36.8 mg, 37.8 umol) in dioxane (6 mL) was stirred at 110° C. for 16 hrs under N₂ atmosphere. On completion, the reaction mixture was diluted with EA (50 mL), filtered and concentrated in vacuo. The residue was purified by prep-HPLC (column: Phenomenex luna C18 150*25 mm*10 um; mobile phase: [water(0.225% FA)-ACN]; B %: 40%-70%, 10 min) to give the title compound (153 mg, 43% yield) as yellow solid. ¹HNMR(400 MHz, DMSO-d₆) δ 9.62 (d, J=6.0 Hz, 1H), 8.55 (d, J=2.8 Hz, 1H), 7.78-7.72 (m, 1H), 7.67-7.63 (m, 1H), 7.45 (t, J=8.4 Hz, 1H), 7.28-7.23 (m, 2H), 6.88 (d, J=8.0 Hz, 2H), 4.83 (s, 2H), 3.99-3.84 (m, 2H), 3.80-3.74 (m, 1H), 3.73 (s, 3H), 3.69-3.52 (m, 2H), 3.46-3.39 (m, 1H), 3.21 (m, 1H), 3.19-3.07 (m, 2H), 3.00-0.92 (m, 1H), 2.80-2.72 (m, 1H), 1.45 (s, 9H), 0.84-0.80 (m, 3H).

### Step 2—1-[8-[(2R)-2-methylpiperazin-1-yl]-4-isoquinolyl]hexahydropyrimidine-2,4-dione

To a solution of tert-butyl (3R)-4-[4-[3-[(4-methoxyphenyl)methyl]-2,4-dioxo-hexahydropyrimidin-1-yl]-8-isoquinolyl]-3-methyl-piperazine-1-carboxylate (75.0 mg, 134 umol) in TFA (770 mg, 6.75 mmol) was added TfOH (170 mg, 1.13 mmol). The reaction mixture was then stirred at 70° C. for 2 hrs. On completion, the reaction mixture was concentrated in vacuo to give the title compound (60.0 mg, 99% yield, TFA) as red oil. LC-MS (ESI⁺) m/z 340.3 (M+H)⁺.

### Step 3—Tert-butyl (3R)-4-[4-(2,4-dioxohexahydropyrimidin-1-yl)-8-isoquinolyl]-3-methyl-piperazine-1-carboxylate

A mixture of 1-[8-[(2R)-2-methylpiperazin-1-yl]-4-isoquinolyl]hexahydropyrimidine-2,4-dione (60.0 mg, 132 umol, TFA), TEA (26.7 mg, 264 umol) and Boc₂O (43.3 mg, 198 umol) in DCM (1 mL) was stirred at 25° C. for 1 hr. On completion, the reaction mixture was diluted with DCM (20 mL) and washed with water (15 mL×3). The organic layer was dried over Na₂SO₄ and filtered. The filtrate was concentrated in vacuo to give the title compound (58.0 mg, 99% yield) as yellow solid. ¹H NMR (400 MHz, DMSO-d₆) δ 10.36-10.29 (m, 1H), 7.41-7.33 (m, 1H), 7.30-7.23 (m, 1H), 7.16-6.99 (m, 3H), 3.94-3.78 (m, 2H), 3.66-3.55 (m, 2H), 3.26-3.07 (m, 3H), 2.78-2.69 (m, 2H), 2.68-2.57 (m, 2H), 1.52 (s, 9H), 1.45 (s, 3H).

### Step 4—1-[8-[(2R)-2-methylpiperazin-1-yl]-4-isoquinolyl]hexahydropyrimidine-2,4-dione

To a solution of tert-butyl (3R)-4-[4-(2,4-dioxohexahydropyrimidin-1-yl)-8-isoquinolyl]-3-methyl-piperazine-1-carboxylate (38 mg, 86.4 umol) in DCM (1 mL) was added TFA (585 mg, 5.13 mmol) at 25° C. The reaction mixture was stirred at 25° C. for 1 hr. On completion, the reaction mixture was concentrated in vacuo to give title compound (39.1 mg, 99% yield, TFA) as brown oil. LC-MS (ESI⁺) m/z 340.2 (M+H)⁺.

### 2-(2,6-Dioxo-3-piperidyl)-4-[[3-[methyl-[3-(methylamino)propyl]amino]cyclobutyl]amino]isoindoline-1,3-dione (Intermediate CAT)

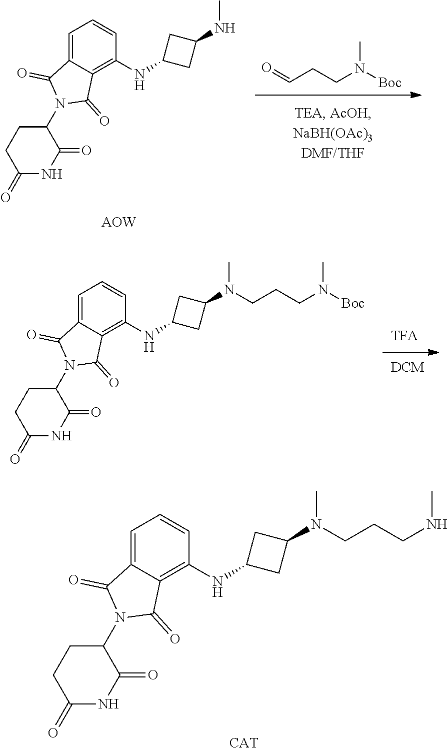 [SMILES: CN[C@H]1C[C@H](Nc2cccc3c2C(=O)N(C2CCC(=O)NC2=O)C3=O)C1 ; CN(CCC=O)C(=O)OC(C)(C)C ; "AOW" ; CN(CCCN(C)[C@H]1C[C@H](Nc2cccc3c2C(=O)N(C2CCC(=O)NC2=O)C3=O)C1)C(=O)OC(C)(C)C ; CNCCCN(C)[C@H]1C[C@H](Nc2cccc3c2C(=O)N(C2CCC(=O)NC2=O)C3=O)C1]

### Step 1—Tert-butyl N-[3-[[3-[[2-(2,6-dioxo-3-piperidyl)-1,3-dioxo-isoindolin-4-yl]amino]cyclobutyl]-methyl-amino]propyl]-N-methyl-carbamate

To a solution of 2-(2,6-dioxo-3-piperidyl)-4-[[3-(methylamino)cyclobutyl]amino]isoindoline-1,3-dione (100 mg, 212 umol, Intermediate AOW) and tert-butyl N-methyl-N-(3-oxopropyl)carbamate (39.8 mg, 212 umol, CAS #273757-11-2) in mixture solvent THF (4 mL) and DMF (1 mL) was added TEA (43.0 mg, 425 umol), then the mixture was stirred at 25° C. for 5 mins. Next, AcOH (38.3 mg, 637 umol) was added to the solution, and the mixture was stirred at 25° C. for 25 mins. Then NaBH(OAc)₃ (90.1 mg, 425 umol) was added to the solution, and the mixture and stirred at 25° C. for 1.5 hours. On completion, the mixture was quenched with H₂O (0.5 mL) and concentrated in vacuo. The crude product was purified by prep-HPLC (column: Phenomenex Luna C18 150*25 mm*10 um; mobile phase: [water (FA)-ACN]; B %: 12%-42%, 10.5 min) to give the title compound (112 mg, 99% yield) as a yellow solid. ¹H NMR (400 MHz, DMSO-d₆) δ 11.11 (s, 1H), 7.66-7.58 (m, 1H), 7.12 (d, J=7.2 Hz, 1H), 6.90 (d, J=8.8 Hz, 1H), 6.65-6.57 (m, 1H), 5.06 (dd, J=5.6, 12.8 Hz, 1H), 4.15-4.10 (m, 1H), 3.20 (t, J=7.2 Hz, 3H), 3.00-2.82 (m, 2H), 2.78 (s, 3H), 2.64-2.60 (m, 1H), 2.59-2.51 (m, 5H), 2.47-2.37 (m, 2H), 2.31-2.21 (m, 2H), 2.07-2.00 (m, 1H), 1.83-1.69 (m, 2H), 1.39 (s, 9H). LC-MS (ESI⁺) m/z 528.2 (M+H)⁺.

### Step 2—2-(2, 6-Dioxo-3-piperidyl)-4-[[3-[methyl-[3-(methylamino) propyl]amino]cyclobutyl] amino]isoindoline-1, 3-dione

To a solution of tert-butyl N-[3-[[3-[[2-(2, 6-dioxo-3-piperidyl)-1, 3-dioxo-isoindolin-4-yl]amino]cyclobutyl]-methyl-amino]propyl]-N-methyl-carbamate (50 mg, 94.7 umol) in DCM (2 mL) was added TFA (154 mg, 1.35 mmol). The mixture was then stirred at 25° C. for 2 hrs. On completion, the mixture was concentrated in vacuo to give the title compound (51 mg, 99% yield, TFA) as yellow oil. LC-MS (ESI⁺) m/z 428.3 (M+H)⁺.

### N-[2-(4-formylcyclohexyl)-6-(1-hydroxy-1-methyl-ethyl)indazol-5-yl]-6-methyl-pyridine-2-carboxamide (Intermediate CAU)

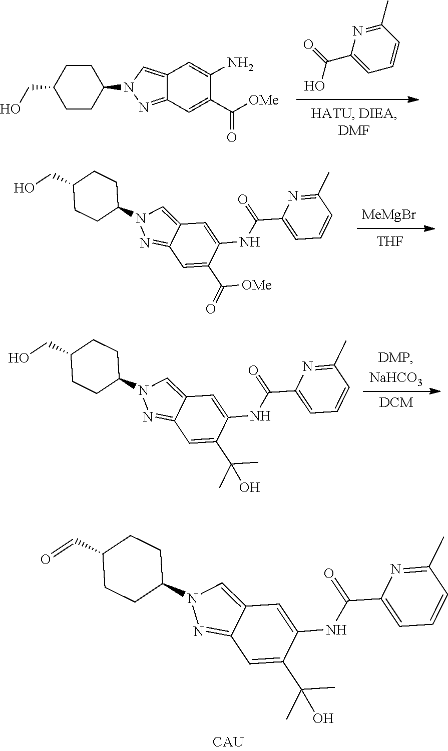

### Step 1—Methyl 2-[4-(hydroxymethyl)cyclohexyl]-5-[(6-methylpyridine-2-carbonyl)amino]indazole-6-carboxylate

A mixture of 6-methylpyridine-2-carboxylic acid (162 mg, 1.19 mmol, CAS #934-60-1) in DMF (2 mL) was added DIEA (383 mg, 2.97 mmol) and HATU (752 mg, 1.98 mmol) at 0° C. The mixture was stirred at 0° C. for 0.5 hr. Then methyl 5-amino-2-[4-(hydroxymethyl)cyclohexyl]indazole-6-carboxylate (300 mg, 988 umol, synthesized via Steps 1-3 of Intermediate BXI) in DMF (2 mL) was added the mixture at 0° C. The mixture was then stirred at 25° C. for 2 hrs. On completion, the reaction mixture was concentrated under reduced pressure to give a residue. The residue was purified by prep-HPLC (column: YMC Triart C18 250*50 mm*7 um; mobile phase: [water(NH₄HCO₃)-ACN]; B %: 48%-78%, 10 min) to give the title compound (230 mg, 54% yield, FA salt) as white liquid. LC-MS (ESI⁺) m/z 423.0 (M+H)⁺. ¹H NMR (400 MHz, DMSO-d₆) δ 12.59 (s, 1H), 9.07 (s, 1H), 8.55 (s, 1H), 8.45 (s, 1H), 8.00-7.93 (m, 2H), 7.55-7.53 (m, 1H), 4.53-4.74 (m, 2H), 3.98 (s, 3H), 3.30-3.27 (m, 2H), 2.65 (s, 3H), 2.52 (d, J=1.6 Hz, 2H), 2.18 (d, J=9.2 Hz, 2H), 1.92 (d, J=10.8 Hz, 2H), 1.50 (s, 1H), 1.25-1.09 (m, 2H).

### Step 2—N-[2-[4-(hydroxymethyl)cyclohexyl]-6-(1-hydroxy-1-methyl-ethyl)indazol-5-yl]-6-methyl-pyridine-2-carboxamide

A mixture of methyl 2-[4-(hydroxymethyl)cyclohexyl]-5-[(6-methylpyridine-2-carbonyl)amino]indazole-6-carboxylate (230 mg, 544 umol) in THF (20 mL) was degassed and purged with N₂ three times and the mixture was added MeMgBr (3 M, 3.63 mL). Then the mixture was stirred at 25° C. for 4 hrs under N₂ atmosphere. On completion, the reaction mixture was quenched by addition NH₄Cl aq. (10 mL) at 25° C., and then extracted with EA(3×50 mL). The combined organic layers were washed with NaHCO₃ aq. (2×25 mL) and brine (2×25 mL), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; B %: 27%-57%, 10.5 min) to give the title compound (150 mg, 65% yield) as a white solid. LC-MS (ESI⁺) m/z 445.0 (M+Na)+, ¹H NMR (400 MHz, DMSO-d₆) δ 12.38 (s, 1H), 8.69 (s, 1H), 8.33 (s, 1H), 7.99-7.88 (m, 2H), 7.56 (s, 1H), 7.49 (d, J=0.8, 7.2 Hz, 1H), 5.90 (s, 1H), 4.58-4.31 (m, 2H), 3.30 (s, 2H), 2.60 (s, 3H), 2.19-2.08 (m, 2H), 1.90 (d, J=10.4 Hz, 4H), 1.63 (s, 6H), 1.48 (s, 1H), 1.22-1.07 (m, 2H).

### Step 3—N-[2-(4-formylcyclohexyl)-6-(1-hydroxy-1-methyl-ethyl)indazol-5-yl]-6-methyl-pyridine-2-carboxamide

To a mixture of N-[2-[4-(hydroxymethyl)cyclohexyl]-6-(1-hydroxy-1-methyl-ethyl)indazol-5-yl]-6-methyl-pyridine-2-carboxamide (50.0 mg, 118 umol) and NaHCO₃ (49.7 mg, 591 umol,) in THF (3 mL) was added DMP (75.2 mg, 177 umol) at 0° C. The reaction mixture was then stirred at 25° C. for 5 hrs. On completion, the reaction mixture was diluted with saturated Na₂S₂O₃ aq. (15 mL) and saturated NaHCO₃ aqueous (15 mL). The mixture was diluted with water (20 mL) and extracted with DCM (3×20 mL). The organic layer was washed with saturated NaHCO₃ aqueous (2×10 mL) and brine (2×10 mL). The organic layer was dried over anhydrous sodium sulfate and filtered to give the title compound (90 mg, 90% yield) as a white solid. ¹H NMR (400 MHz, DMSO-d₆) δ 12.39 (s, 1H), 9.65 (s, 1H), 8.70 (s, 1H), 8.34 (s, 1H), 8.00-7.89 (m, 2H), 7.57 (s, 1H), 7.49 (d, J=7.2 Hz, 1H), 5.90 (s, 1H), 4.48-4.40 (m, 1H), 2.60 (s, 3H), 2.46-2.39 (m, 1H), 2.22-2.18 (m, 2H), 2.11 (d, J=11.6 Hz, 2H), 2.03-1.92 (m, 2H), 1.63 (s, 6H), 1.50-1.39 (m, 2H).

### 3-[1-(2,6-Dioxo-3-piperidyl)-3-methyl-2-oxo-benzimidazol-4-yl]cyclobutanecarbaldehyde (Intermediate CAV)

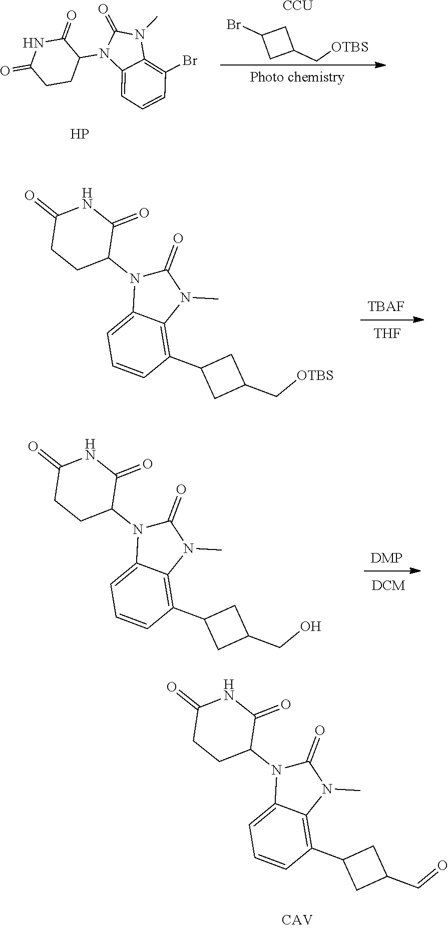

### Step 1—3-[4-[3-[[Tert-butyl(dimethyl)silyl]oxymethyl]cyclobutyl]-3-methyl-2-oxo-benzimidazol-1-yl]piperidine-2,6-dione

To an 15 mL vial equipped with a stir bar was added 3-(4-bromo-3-methyl-2-oxo-benzimidazol-1-yl)piperidine-2,6-dione (745 mg, 2.20 mmol, Intermediate HP), (3-bromocyclobutyl)methoxy-tert-butyl-dimethyl-silane (800 mg, 2.86 mmol, Intermediate CCU), Ir[dF(CF₃)ppy]₂(dtbpy)(PF₆) (24.7 mg, 22.0 umol), NiCl₂.dtbbpy (4.38 mg, 11.0 umol), TTMSS (680 uL, 2.20 mmol), and 2,6-lutidine (513 uL, 4.41 mmol) in DME (2 mL). The vial was sealed and placed under nitrogen and the reaction was stirred and irradiated with a 10 W blue LED lamp (3 cm away), with cooling water to keep the reaction temperature at 25° C. for 14 hr. On completion, the mixture was filtered and the filtrate was washed with water (50 mL) and extracted with EA (30 mL×3). The combined organic layer was dried over anhydrous Na₂SO₄, then concentrated in vacuo to give a residue. The residue was purified by reverse-phase (0.1% FA condition) to give the title compound (335 mg, 33% yield) as a yellow solid. ¹H NMR (400 MHz, CDCl₃-d) δ 8.75 (s, 1H), 7.24-7.12 (m, 1H), 7.08-7.00 (m, 1H), 6.68 (dd, J=5.6, 7.2 Hz, 1H), 5.23 (dd, J=5.2, 12.2 Hz, 1H), 4.11 (quin, J=8.0 Hz, 1H), 3.75 (d, J=6.0 Hz, 1H), 3.69 (s, 1H), 3.63 (s, 2H), 3.60 (d, J=5.2 Hz, 1H), 2.92-2.79 (m, 1H), 2.84-2.79 (m, 1H), 2.76-2.67 (m, 1H), 2.58-2.46 (m, 1H), 2.41-2.31 (m, 2H), 2.31-2.23 (m, 1H), 2.21-2.09 (m, 2H), 0.93-0.93 (m, 1H), 0.93 (d, J=8.0 Hz, 8H), 0.09 (d, J=11.2 Hz, 5H). LC-MS (ESI⁺) m/z 458.4 (M+H)⁺.

### Step 2—3-[4-[3-(Hydroxymethyl)cyclobutyl]-3-methyl-2-oxo-benzimidazol-1-yl]piperidine-2,6-dione

To a solution of 3-[4-[3-[[tert-butyl(dimethyl)silyl]oxymethyl]cyclobutyl]-3-methyl-2-oxo-benzimidazol-1-yl]piperidine-2,6-dione (100 mg, 218 umol) in THF (1 mL) was added TBAF (1 M, 218 uL), then the mixture was stirred at 25° C. for 1 hour. On completion, the mixture was washed with water (30 mL) and extracted with EA (20 mL×3). The combined organic layer was dried over anhydrous Na₂SO₄, then concentrated in vacuo to give a residue. The residue was purified by column chromatography to give the title compound (70.0 mg, 93% yield) as a white solid. ¹H NMR (400 MHz, CDCl₃-d) δ 8.06 (s, 1H), 7.15-7.05 (m, 2H), 6.71-6.66 (m, 1H), 5.25-5.15 (m, 1H), 4.02-3.89 (m, 1H), 3.70 (s, 3H), 3.68-3.63 (m, 3H), 2.99-2.90 (m, 1H), 2.88-2.82 (m, 1H), 2.81-2.70 (m, 2H), 2.56 (d, J=7.2 Hz, 1H), 2.48-2.41 (m, 2H), 2.31-2.18 (m, 2H). LC-MS (ESI⁺) m/z 344.1 (M+H)⁺.

### Step 3—3-[1-(2,6-Dioxo-3-piperidyl)-3-methyl-2-oxo-benzimidazol-4-yl]cyclobutanecarbaldehyde

To a solution of 3-[4-[3-(hydroxymethyl)cyclobutyl]-3-methyl-2-oxo-benzimidazol-1-yl]piperidine-2,6-dione (100 mg, 291 umol) in DCM (2 mL) was added DMP (135 uL, 436 umol), then the mixture was stirred at 25° C. for 1 hour. On completion, the mixture was quenched with sodium thiosulfate pentahydrate (5 mL) and NaHCO₃ (5 mL), then extracted with DCM (30 mL×3). The combined organic layer was dried over anhydrous Na₂SO₄, then concentrated in vacuo to give the title compound (95.0 mg, 95% yield) as a white solid. LC-MS (ESI⁺) m/z 342.1 (M+H)⁺.

### 2-(2,6-dioxo-3-piperidyl)-4-[[3-[3-(methylamino)propoxy]cyclobutyl]amino] isoindoline-1,3-dione (Intermediate AOQ)

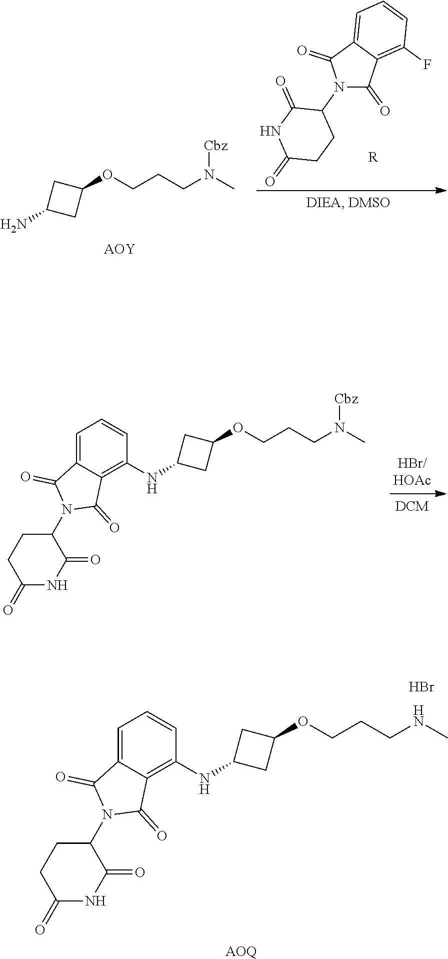

### Step 1—Benzyl N-[3-[3-[[2-(2,6-dioxo-3-piperidyl)-1,3-dioxo-isoindolin-4-yl]amino]cyclobutoxy]propyl]-N-methyl-carbamate

To a solution of benzyl N-[3-(3-aminocyclobutoxy)propyl]-N-methyl-carbamate (1.30 g, 3.95 mmol, HCl salt, Intermediate AOY) and 2-(2,6-dioxo-3-piperidyl)-4-fluoro-isoindoline-1,3-dione (928 mg, 3.36 mmol, Intermediate R) in DMSO (15 mL) was added DIPEA (2.55 g, 19.7 mmol). The reaction mixture was stirred at 130° C. for 3 hrs. On completion, the reaction mixture was diluted with water (50 mL) and extracted with EA (3×70 mL). The combined organic layers were washed with brine (50 mL), dried over Na₂SO₄, filtered and concentrated in vacuo. The residue was purified by reverse phase (0.1% FA) to give the title compound (810 mg, 37% yield) as yellow solid. ¹H NMR (400 MHz, CDCl₃) δ 8.02 (s, 1H), 7.54-7.47 (m, 1H), 7.42-7.28 (m, 5H), 7.14 (d, J=7.2 Hz, 1H), 6.80-6.64 (m, 1H), 6.36-6.22 (m, 1H), 5.14 (s, 2H), 4.99-4.87 (m, 1H), 4.24-4.05 (m, 2H), 3.47-3.28 (m, 4H), 2.95 (s, 3H), 2.93-2.84 (m, 1H), 2.84-2.70 (m, 2H), 2.57-2.35 (m, 2H), 2.28-2.09 (m, 3H), 1.90-1.74 (m, 2H).

### Step 2—2-(2,6-Dioxo-3-piperidyl)-4-[[3-[3-(methylamino)propoxy]cyclobutyl]amino]isoindoline-1,3-dione

To a solution of benzyl N-[3-[3-[[2-(2,6-dioxo-3-piperidyl)-1,3-dioxo-isoindolin-4-yl]amino]cyclobutoxy]propyl]-N-methyl-carbamate (0.80 g, 1.46 mmol) in DCM (10 mL) was added HBr/AcOH (1.46 mmol, 10 mL, 30% solution). The reaction mixture was stirred at 20° C. for 1 hour. On completion, the reaction mixture was dried by nitrogen. The residue was diluted with ACN/H₂O=1/1 (100 mL) and lyophilizated to give the title compound (722 mg, 99% yield, HBr salt) as yellow solid. ¹H NMR (400 MHz, DMSO-d₆) δ 11.09 (s, 1H), 8.32 (s, 2H), 7.60 (dd, J=7.2, 8.4 Hz, 1H), 7.09 (d, J=7.0 Hz, 1H), 6.91 (d, J=8.4 Hz, 1H), 6.68-6.25 (m, 1H), 5.06 (dd, J=5.6, 12.8 Hz, 1H), 4.22-4.11 (m, 2H), 3.38 (t, J=6.0 Hz, 2H), 3.04-2.80 (m, 3H), 2.63-2.55 (m, 4H), 2.54-2.52 (m, 1H), 2.44-2.33 (m, 2H), 2.29-2.18 (m, 2H), 2.12-1.97 (m, 1H), 1.90-1.76 (m, 2H).

### N-[2-(4-formylcyclohexyl)-6-(1-hydroxy-1-methyl-ethyl)indazol-5-yl]pyridine-2-carboxamide (Intermediate CAW)

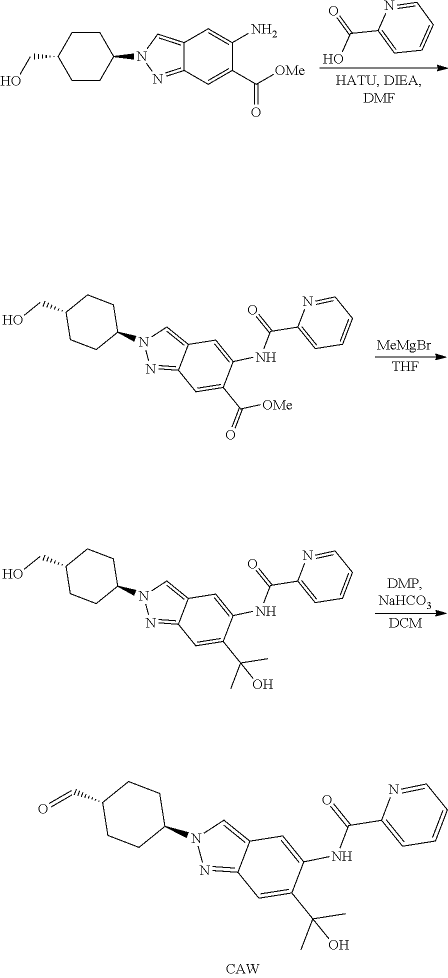

### Step 1—Methyl 2-[4-(hydroxymethyl)cyclohexyl]-5-(pyridine-2-carbonylamino)indazole-6-carboxylate

To a mixture of pyridine-2-carboxylic acid (146 mg, 1.19 mmol, CAS #636-80-6) in DMF (2 mL) was added DIEA (383 mg, 2.97 mmol) and HATU (752 mg, 1.98 mmol) at 0° C. The mixture was stirred at 0° C. for 0.5 hr. Then methyl 5-amino-2-[4-(hydroxymethyl)cyclohexyl]indazole-6-carboxylate (300 mg, 988 umol, synthesized via Steps 1-3 of Intermediate BXI) in DMF (2 mL) was added the mixture at 0° C. The mixture was stirred at 25° C. for 2 hrs. On completion, the reaction mixture was concentrated under reduced pressure to give a residue. The residue was purified by prep-HPLC (column: YMC-Triart Prep C18 150*40 mm*7 um; mobile phase: [water(NH4HCO3)-ACN]; B %: 39%-69%, 8 min) to give the title compound (220 mg, 53% yield) as yellow solid. LC-MS (ESI⁺) m/z 409.0 (M+H)⁺. ¹H NMR (400 MHz, DMSO-d₆) δ 12.63 (s, 1H), 9.09 (s, 1H), 8.79 (d, J=4.8 Hz, 1H), 8.56 (s, 1H), 8.47 (s, 1H), 8.20 (d, J=7.6 Hz, 1H), 8.10-8.06 (m, 1H), 7.69-7.66 (m, 1H), 4.56-4.45 (m, 2H), 3.97 (s, 3H), 3.31-3.25 (m, 3H), 2.19-2.16 (m, 2H), 1.93-1.90 (m, 3H), 1.50-1.49 (m, 1H), 1.26-1.09 (m, 2H).

### Step 2—N-[2-[4-(hydroxymethyl)cyclohexyl]-6-(1-hydroxy-1-methyl-ethyl)indazol-5-yl]pyridine-2-carboxamide

A mixture of methyl 2-[4-(hydroxymethyl)cyclohexyl]-5-(pyridine-2-carbonylamino)indazole-6-carboxylate (220 mg, 538 umol) in THF (50 mL) was degassed and purged with N₂ three times Next, MeMgBr (3 M, 3.59 mL) was added to the reaction mixture and then the mixture was stirred at 25° C. for 4 hrs under N₂ atmosphere. On completion, the reaction mixture was quenched by addition NH₄Cl aq. 10 mL at 25° C., and then extracted with EA (3×50 mL). The combined organic layers were washed with NaHCO₃ aq. (2×25 mL) and brine (2×25 mL), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; B %: 23%-53%, 10.5 mm) to give the title compound (131 mg, 59% yield) as a white solid. LC-MS (ESI⁺) m/z 408.9 (M+H)⁺, ¹H NMR (400 MHz, DMSO-d₆) δ 12.32 (s, 1H), 8.69-8.67 (m, 2H), 8.34 (s, 1H), 8.26-7.97 (m, 2H), 7.73-7.47 (m, 2H), 6.02 (s, 1H), 4.61-4.27 (m, 2H), 3.30-3.23 (m, 2H), 2.13 (s, 2H), 1.90 (d, J=10.4 Hz, 4H), 1.61 (s, 6H), 1.48-1.47 (m, 1H), 1.25-1.08 (m, 2H).

### Step 3—N-[2-(4-formylcyclohexyl)-6-(1-hydroxy-1-methyl-ethyl)indazol-5-yl]pyridine-2-carboxamide

To a mixture of N-[2-[4-(hydroxymethyl)cyclohexyl]-6-(1-hydroxy-1-methyl-ethyl)indazol-5-yl]pyridine-2-carboxamide (50.0 mg, 122 umol) and NaHCO₃ (51.4 mg, 612 umol) in THF (3 mL) was added DMP (129 mg, 306 umol) at 0° C. The reaction mixture was stirred at 25° C. for 8 hrs. On completion, the reaction mixture was diluted with saturated Na₂S₂O₃ aq. (15 mL) and saturated NaHCO₃ aqueous (15 mL). Then the mixture was diluted with water (20 mL) and extracted with DCM (3×20 mL). The organic layer was washed with saturated NaHCO₃ aqueous (2×10 mL) and brine (2×10 mL). The organic layer was dried over anhydrous sodium sulfate and filtered to give the title compound (99 mg, 95% yield) as a white solid. LC-MS (ESI⁺) m/z 407.1 (M+H)⁺, ¹H NMR (400 MHz, DMSO-d₆) δ 12.33 (s, 1H), 9.65 (s, 1H), 8.74-8.65 (m, 2H), 8.35 (s, 1H), 8.19 (d, J=7.6 Hz, 1H), 8.08-8.04 (m, 1H), 7.67-7.60 (m, 1H), 7.56 (s, 1H), 6.02 (s, 1H), 4.49-4.41 (m, 1H), 2.46-2.40 (m, 1H), 2.22-2.18 (m, 2H), 2.11 (br d, J=11.2 Hz, 2H), 2.04-1.99 (m, 1H), 1.98-1.92 (m, 1H), 1.62 (s, 6H), 1.45 (q, J=4.0, 13.2 Hz, 2H).

### 3-[4-(2,5-Dihydro-1H-pyrrol-3-yl)-3-methyl-2-oxo-benzimidazol-1-yl]piperidine-2,6-dione (Intermediate CAX)

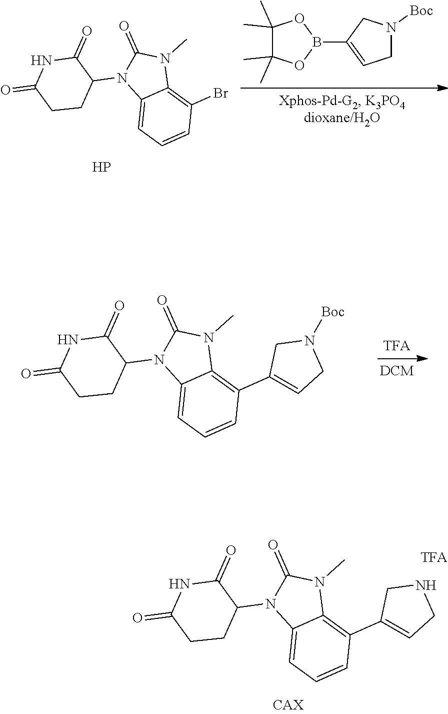

### Step 1—Tert-butyl 3-[1-(2, 6-dioxo-3-piperidyl)-3-methyl-2-oxo-benzimidazol-4-yl]-2, 5-dihydropyrrole-1-carboxylate

A mixture of 3-(4-bromo-3-methyl-2-oxo-benzimidazol-1-yl)piperidine-2,6-dione (2 g, 5.91 mmol, Intermediate HP), tert-butyl 3-(4,4,5,5-tetramethyl-1,3,2-dioxaborolan-2-yl)-2,5-dihydropyrrole-1-carboxylate (2.09 g, 7.10 mmol, CAS #212127-83-8), XPHOS-PD-G2 (465 mg, 591 umol), K₃PO₄ (3.77 g, 17.7 mmol) in dioxane (20 mL) and H₂O (4 mL) was degassed and purged with N₂ three times. Then the mixture was stirred at 80° C. for 2 hrs under N₂ atmosphere. On completion, the reaction mixture was filtered to give the filtrate. The filtrate was diluted with EA (100 mL) and washed with H₂O (2×30 mL). The combined organic layer was dried over Na₂SO₄, filtered and concentrated in vacuo. The residue was purified by prep-HPLC (1% FA condition) to give the title compound (2.5 g, 99% yield) as a yellow solid. ¹H NMR (400 MHz, CDCl₃) δ 8.73 (s, 1H), 7.09-6.97 (m, 1H), 6.90 (d, J=7.6 Hz, 1H), 6.77 (d, J=7.6 Hz, 1H), 5.91-5.63 (m, 1H), 5.25 (d, J=5.2, 12.4 Hz, 1H), 4.52-4.10 (m, 4H), 3.42 (d, J=3.2 Hz, 3H), 2.96-2.67 (m, 4H), 1.49 (d, J=8.1 Hz, 9H). LC-MS (ESI⁺) m/z 427.2 (M+H)⁺.

### Step 2—3-[4-(2,5-Dihydro-1H-pyrrol-3-yl)-3-methyl-2-oxo-benzimidazol-1-yl]piperidine-2,6-dione

To a solution of tert-butyl 3-[1-(2,6-dioxo-3-piperidyl)-3-methyl-2-oxo-benzimidazol-4-yl]-2,5-dihydropyrrole-1-carboxylate (100 mg, 234 umol) in DCM (1 mL) was added TFA (0.3 mL). The mixture was stirred at 25° C. for 2 hrs. On completion, the reaction was concentrated in vacuo to give the title compound (103 mg, 99% yield, TFA) as yellow oil. LC-MS (ESI⁺) m/z 327.1 (M+H)⁺.

### 3-(3-methyl-2-oxo-5-piperazin-1-yl-benzimidazol-1-yl)piperidine-2,6-dione (Intermediate CAY)

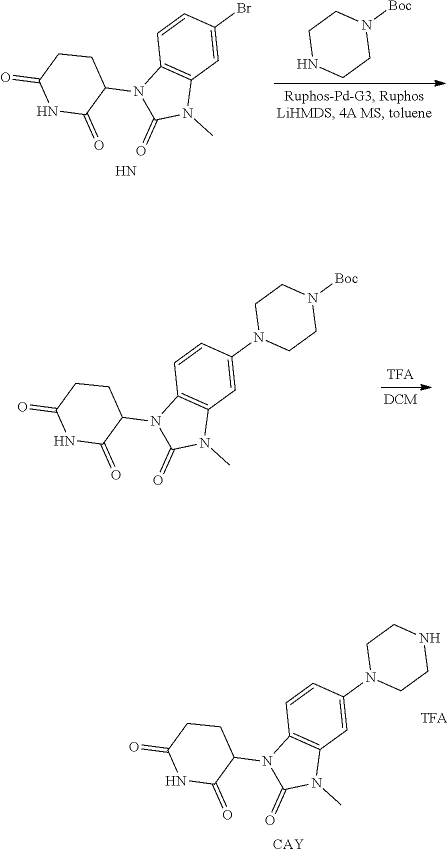

### Step 1—Tert-butyl 4-[1-(2,6-dioxo-3-piperidyl)-3-methyl-2-oxo-benzimidazol-5-yl]piperazine-1-carboxylate

To a solution of 3-(5-bromo-3-methyl-2-oxo-benzimidazol-1-yl)piperidine-2,6-dione (500 mg, 1.48 mmol, Intermediate HN) and tert-butyl piperazine-1-carboxylate (413 mg, 2.22 mmol, CAS #143238-38-4) in toluene (4 mL) was added RuPhos Pd G3 (247 mg, 295 umol), RuPhos (138 mg, 295 umol) and LiHMDS (1 M, 6 mL). Then the mixture was stirred at 100° C. for 16 hr under N₂ atmosphere. On completion, the reaction mixture was quenched with FA (12 mL) and concentrated in vacuo. The mixture was purified by column chromatography (SiO₂, DCM:EtOH=10:1) and concentrated in vacuo. The residue was purified by prep-HPLC (column: Phenomenex Luna C18 150*25 mm*10 um; mobile phase: [water(FA)-ACN]; B %: 24%-54%, 10.5 mins) concentrated in vacuo to give the title compound (150 mg, 23% yield) as brown solid. ¹H NMR (400 MHz, DMSO-d₆) δ 11.06 (s, 1H), 6.96 (d, J=8.4 Hz, 1H), 6.87 (d, J=2.0 Hz, 1H), 6.66 (dd, J=2.4, 8.4 Hz, 1H), 5.29 (dd, J=5.6, 12.8 Hz, 1H), 3.51-3.44 (m, 4H), 3.30 (s, 3H), 3.06-3.00 (m, 4H), 2.93-2.84 (m, 1H), 2.73-2.65 (m, 1H), 2.59 (d, J=2.4 Hz, 1H), 2.02-1.94 (m, 1H), 1.42 (s, 9H); LC-MS (ESI⁺) m/z 444.2 (M+H)⁺.

### Step 2—3-(3-methyl-2-oxo-5-piperazin-1-yl-benzimidazol-1-yl)piperidine-2,6-dione

To a solution of tert-butyl 4-[1-(2,6-dioxo-3-piperidyl)-3-methyl-2-oxo-benzimidazol-5-yl]piperazine-1-carboxylate (40 mg, 90.1 umol) in DCM (0.5 mL) was added TFA (154 mg, 1.35 mmol), then the mixture was stirred at 25° C. for 0.5 hr. On completion, the reaction mixture was concentrated in vacuo to give the title compound (40 mg, 96% yield, TFA) as red solid. LC-MS (ESI⁺) m/z 344.2 (M+H)⁺.

### 6-(5-fluoropyrrolo[2,3-b]pyridin-1-yl)-N-(4-formylcyclohexyl)-4-(isopropylamino)pyridine-3-carboxamide (Intermediate CAZ)

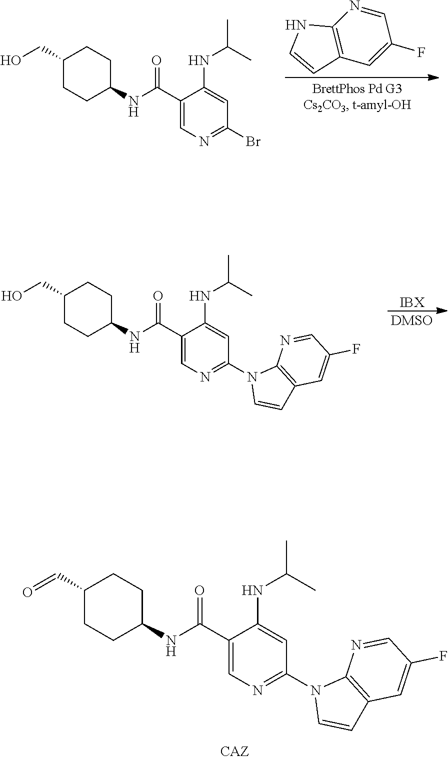

### Step 1—6-(5-Fluoropyrrolo[2,3-b]pyridin-1-yl)-N-[4-(hydroxymethyl)cyclohexyl]-4-(isopropylamino) pyridine-3-carboxamide

To a solution of 6-bromo-N-[4-(hydroxymethyl)cyclohexyl]-4-(isopropylamino) pyridine-3-carboxamide (250 mg, 675 umol, synthesized via Steps 1-4 of Intermediate BVU) and 5-fluoro-1H-pyrrolo[2,3-b]pyridine (110 mg, 810 umol, CAS #866319-00-8) in t-amyl-OH (1 mL) was added Cs₂CO₃ (439 mg, 1.35 mmol) and BrettPhos Pd G₃ (61.2 mg, 67.5 umol). Then the mixture was stirred at 100° C. for 16 hrs under N₂ atmosphere. On completion, the reaction mixture was filtered to give the filtrate. The residue was purified by reverse phase (0.1% FA condition) to give the title compound (135 mg, 23% yield) as white solid. ¹H NMR (400 MHz, DMSO-d₆) δ 8.57 (d, J=7.2 Hz, 1H), 8.54 (s, 1H), 8.47 (d, J=4.0 Hz, 1H), 8.40 (dd, J=1.2, 2.8 Hz, 1H), 8.26 (d, J=8.0 Hz, 1H), 8.19 (s, 1H), 8.03 (dd, J=2.8, 9.2 Hz, 1H), 6.74 (d, J=4.0 Hz, 1H), 4.40 (s, 1H), 3.81-3.73 (m, 1H), 3.73-3.64 (m, 1H), 3.23 (d, J=6.0 Hz, 2H), 2.52 (d, J=1.6 Hz, 1H), 1.93-1.83 (m, 2H), 1.79 (d, J=11.6 Hz, 2H), 1.39-1.31 (m, 2H), 1.29 (d, J=6.4 Hz, 6H), 1.05-0.90 (m, 2H); LC-MS (ESI⁺) m/z 426.3 (M+H)⁺.

### Step 2—6-(5-Fluoropyrrolo[2,3-b]pyridin-1-yl)-N-(4-formylcyclohexyl)-4-(isopropylamino) pyridine-3-carboxamide

To a solution of 6-(5-fluoropyrrolo[2,3-b]pyridin-1-yl)-N-[4-(hydroxymethyl) cyclohexyl]-4-(isopropylamino)pyridine-3-carboxamide (35 mg, 82.3 umol) in DMSO (0.5 mL) was added IBX (69.1 mg, 246 umol), then the mixture was stirred at 50° C. for 1 hr. On completion, the mixture was quenched with saturated NaHCO₃ (5 mL) and saturated Na₂S₂O₃ (5 mL), and the mixture was stirred at 25° C. for 0.5 hr. Next, the mixture was diluted with DCM (20 mL) and the organic layer was washed with saturated NaHCO₃ (10 mL×3). The organic layer was separated and washed with saturated NaCl (10 mL), the organic layer was dried over Na₂SO₄, filtered and concentrated in vacuo to give the title compound (32 mg, 91% yield) as yellow solid. ¹H NMR (400 MHz, DMSO-d₆) δ 9.59 (d, J=0.8 Hz, 1H), 8.58-8.51 (m, 2H), 8.47 (d, J=4.0 Hz, 1H), 8.40 (dd, J=1.2, 2.4 Hz, 1H), 8.32 (d, J=7.6 Hz, 1H), 8.19 (s, 1H), 8.03-7.76 (m, 1H), 6.75 (d, J=3.6 Hz, 1H), 3.82-3.75 (m, 1H), 3.75-3.66 (m, 1H), 2.55-2.52 (m, 2H), 2.26-2.12 (m, 1H), 2.05-1.88 (m, 4H), 1.42-1.32 (m, 2H), 1.29 (d, J=6.4 Hz, 6H); LC-MS (ESI⁺) m/z 424.3 (M+H)⁺.

### 3-[3-methyl-2-oxo-4-[3-(piperazin-1-ylmethyl)cyclobutyl]benzimidazol-1-yl]piperidine-2,6-dione (Intermediate CBA)

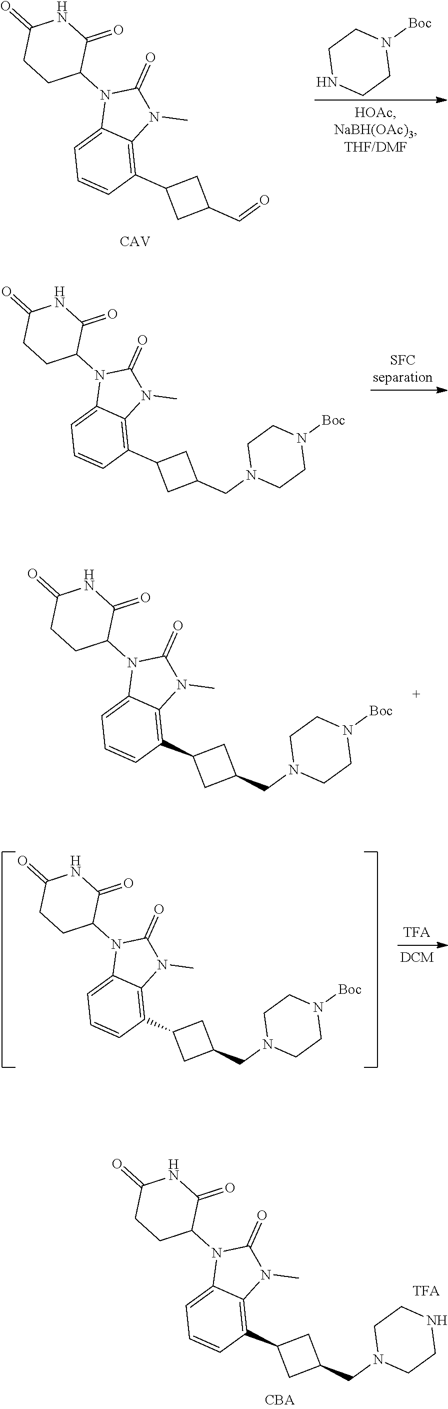

### Step 1—Tert-butyl 4-[[3-[1-(2,6-dioxo-3-piperidyl)-3-methyl-2-oxo-benzimidazol-4-yl]cyclobutyl] methyl]piperazine-1-carboxylate

To a solution of 3-[1-(2,6-dioxo-3-piperidyl)-3-methyl-2-oxo-benzimidazol-4-yl]cyclobutanecarbaldehyde (160 mg, 468 umol, Intermediate CAV) in THF (3 mL) was added tert-butylpiperazine-1-carboxylate (104 mg, 562 umol, CAS #143238-38-4), then HOAc (26.8 uL, 468 umol) was added, then the mixture was stirred at 25° C. for 0.5 hour. Then NaBH(OAc)₃ (149 mg, 703 umol) was added, and the mixture was stirred at 25° C. for 1.5 hours. On completion, the mixture was concentrated in vacuo to give the residue. The residue was purified by prep-HPLC (column: Phenomenex luna C18 150*25 mm*10 um; mobile phase: [water(0.225% FA)-ACN]; B %: 8%-38%, 10 min) to give the title compound (74.0 mg, 30% yield) as a white solid. ¹H NMR (400 MHz, CDCl₃-d) δ 8.14 (s, 1H), 7.13-7.01 (m, 2H), 6.73-6.65 (m, 1H), 5.21 (dd, J=5.2, 12.4 Hz, 1H), 3.68 (s, 3H), 3.64-3.49 (m, 4H), 2.99-2.85 (m, 2H), 2.84-2.47 (m, 10H), 2.35-2.17 (m, 2H), 2.03 (d, J=9.6 Hz, 2H), 1.47 (s, 9H). LC-MS (ESI⁺) m/z 512.4 (M+H)⁺.

### Step 2—Tert-butyl 4-[[3-[1-(2,6-dioxo-3-piperidyl)-3-methyl-2-oxo-benzimidazol-4-yl]cyclobutyl]methyl]piperazine-1-carboxylate and tert-butyl 4-[[3-[1-(2,6-dioxo-3-piperidyl)-3-methyl-2-oxo-benzimidazol-4-yl]cyclobutyl]methyl]piperazine-1-carboxylate

Tert-butyl 4-[[3-[1-(2,6-dioxo-3-piperidyl)-3-methyl-2-oxo-benzimidazol-4-yl]cyclobutyl]methyl]piperazine-1-carboxylate (610 mg) was separated by SFC (column: DAICEL CHIRALPAK IC(250 mm*30 mm, 10 um); mobile phase: [IPA-ACN]; B %: 80%-80%, 6; 40 min) to give the title compound tert-butyl 4-[[3-[1-(2,6-dioxo-3-piperidyl)-3-methyl-2-oxo-benzimidazol-4-yl]cyclobutyl]methyl]piperazine-1-carboxylate (150 mg, 50% yield) as a white solid and the title compound tert-butyl 4-[[3-[1-(2,6-dioxo-3-piperidyl)-3-methyl-2-oxo-benzimidazol-4-yl]cyclobutyl]methyl]piperazine-1-carboxylate (70.0 mg, 23% yield) as a white solid. ¹HNMR (400 MHz, DMSO-d₆) δ 11.09 (s, 1H), 7.09-6.95 (m, 3H), 5.37-5.32 (m, 1H), 4.34-4.04 (m, 3H), 3.99-3.89 (m, 2H), 3.56 (s, 3H), 2.96-2.81 (m, 1H), 2.75-2.58 (m, 2H), 2.46-2.37 (m, 4H), 2.34-2.28 (m, 4H), 2.02-1.94 (m, 1H), 1.91-1.81 (m, 2H), 1.39 (s, 9H).

### Step 3—3-[3-Methyl-2-oxo-4-[3-(piperazin-1-ylmethyl)cyclobutyl]benzimidazol-1-yl]piperidine-2,6-dione

To a solution of tert-butyl 4-[[3-[1-(2,6-dioxo-3-piperidyl)-3-methyl-2-oxo-benzimidazol-4-yl]cyclobutyl]methyl]piperazine-1-carboxylate (50.0 mg, 97.7 umol) in DCM (2 mL) was added TFA (300 uL, 4.05 mmol), then the mixture was stirred at 25° C. for 1 hour. On completion, the residue was concentrated in vacuo to give the title compound (50.0 mg, 97% yield, TFA) as a white solid. LC-MS (ESI⁺) m/z 412.2 (M+H)⁺.

### N-[2-[4-(iodomethyl)cyclohexyl]indazol-5-yl]-6-(trifluoromethyl)pyridine-2-carboxamide (Intermediate CBB)

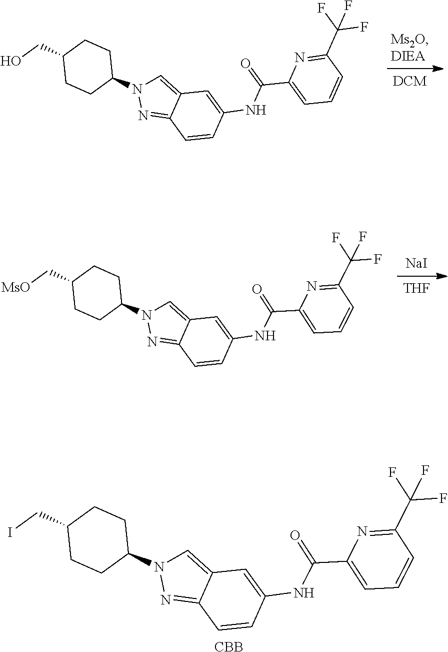

### Step 1—[4-[5-[[6-(Trifluoromethyl)pyridine-2-carbonyl]amino]indazol-2-yl]cyclohexyl]methyl methanesulfonate

To a solution of N-[2-[4-(hydroxymethyl)cyclohexyl]indazol-5-yl]-6-(trifluoromethyl)pyridine-2-carboxamide (100 mg, 239 umol, synthesized via Steps 1-2 of Intermediate BRP) in DCM (5 mL) was added methylsulfonyl methanesulfonate (124 mg, 717 umol) and DIEA (123 mg, 956 umol), then the mixture was stirred at 25° C. for 2 hrs. On completion, the reaction mixture was poured into water (10 mL) and extracted with DCM (10 mL×2). The combined organic layers were dried over Na₂SO₄, filtered and concentrated in vacuo give the title compound (110 mg, 93% yield) as yellow solid. LC-MS (ESI⁺) m/z 497.3 (M+H)⁺.

### Step 2—N-[2-[4-(iodomethyl)cyclohexyl]indazol-5-yl]-6-(trifluoromethyl)pyridine-2-carboxamide

To a solution of [4-[5-[[6-(trifluoromethyl)pyridine-2-carbonyl]amino]indazol-2-yl]cyclohexyl]methyl methanesulfonate (110 mg, 221 umol) in THF (4 mL) was added NaI (99.6 mg, 664 umol), then the mixture was stirred at 60° C. for 16 hrs. On completion, the reaction mixture was poured into 10 mL of water and extracted with EtOAc (10 mL×2). The combined organic layers were washed by saturated brine (10 mL), dried over Na₂SO₄, filtered and concentrated in vacuo give the title compound (110 mg, 94% yield) as yellow solid. LC-MS (ESI⁺) m/z 529.1 (M+H)⁺.

### 3-[4-(3,3-dimethylpiperazin-1-yl)-3-methyl-2-oxo-benzimidazol-1-yl]piperidine-2,6-dione (Intermediate CBC)

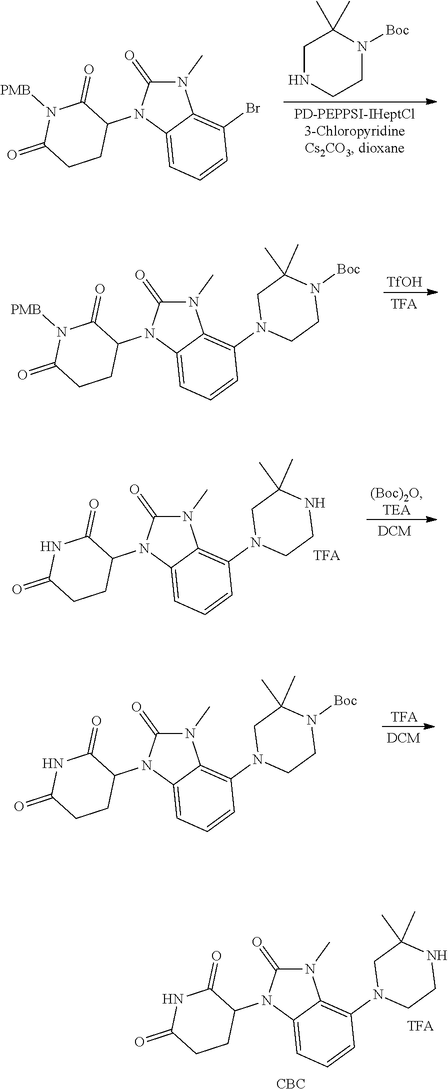

### Step 1—Tert-butyl 4-[1-[1-[(4-methoxyphenyl)methyl]-2,6-dioxo-3-piperidyl]-3-methyl-2-oxo-benzimidazol-4-yl]-2,2-dimethyl-piperazine-1-carboxylate

To a solution of 3-(4-bromo-3-methyl-2-oxo-benzimidazol-1-yl)-1-[(4-methoxyphenyl)methyl]piperidine-2,6-dione (1 g, 2.18 mmol, synthesized via Steps 1-4 of Intermediate HP) and tert-butyl 2,2-dimethylpiperazine-1-carboxylate (701 mg, 3.27 mmol, CAS #674792-07-5) in dioxane (3 mL) was added Cs₂CO₃ (1.42 g, 4.36 mmol) and PD-PEPPSI-IHeptCl 3-Chloropyridine (40 mg, 0.218 mmol), then the solution was stirred at 100° C. for 16 hrs. On completion, the reaction mixture was filtered. The filtrate was diluted with water (10 mL) and extracted with EA (30 mL×2). The combined organic layer was washed with saturated brine (10 mL). The organic layer was dried over Na₂SO₄, filtered and concentrated in vacuo to give a residue. The residue was purified by reverse phase (0.1% FA condition) to give the title compound (590 mg, 46% yield) as brown solid. ¹H NMR (400 MHz, DMSO-d₆) δ 7.21 (d, J=8.8 Hz, 2H), 6.94 (d, J=4.0 Hz, 2H), 6.90-6.74 (m, 3H), 5.51 (dd, J=5.6, 12.8 Hz, 1H), 4.86-4.72 (m, 2H), 3.72 (s, 3H), 3.67 (s, 3H), 3.12-2.96 (m, 2H), 2.90-2.60 (m, 6H), 2.52-2.51 (m, 1H), 2.07-1.97 (m, 1H), 1.56-1.37 (m, 15H).

### Step 2—3-[4-(3,3-Dimethylpiperazin-1-yl)-3-methyl-2-oxo-benzimidazol-1-yl]piperidine-2,6-dione

A solution of tert-butyl 4-[1-[1-[(4-methoxyphenyl)methyl]-2,6-dioxo-3-piperidyl]-3-methyl-2-oxo-benzimidazol-4-yl]-2,2-dimethyl-piperazine-1-carboxylate (500 mg, 845 umol) in TfOH (1 mL) and TFA (5 mL) was stirred at 70° C. for 2 hrs. On completion, the reaction mixture was adjusted to pH=7-8 with TEA and then concentrated in vacuo to give the title compound (300 mg, 96% yield). LC-MS (ESI+) m/z 372.2 (M+H)⁺.

### Step 3—Tert-butyl 4-[1-(2,6-dioxo-3-piperidyl)-3-methyl-2-oxo-benzimidazol-4-yl]-2,2-dimethyl-piperazine-1-carboxylate

To a solution of 3-[4-(3,3-dimethylpiperazin-1-yl)-3-methyl-2-oxo-benzimidazol-1-yl]piperidine-2,6-dione (300 mg, 807 umol) in DCM (5 mL) was added TEA (163 mg, 1.62 mmol) and (Boc)₂O (264 mg, 1.21 mmol), the reaction was stirred at 25° C. for 16 hrs. On completion, the reaction mixture was diluted with water (10 mL) and extracted with DCM (10 mL×2). The organic layer was dried over Na₂SO₄, filtered and concentrated in vacuo to give a residue. The residue was purified by reverse phase (0.1% FA condition) to give the title compound (340 mg, 89% yield) as yellow oil. LC-MS (ESI+) m/z 472.3 (M+H)⁺.

### Step 4—3-[4-(3,3-Dimethylpiperazin-1-yl)-3-methyl-2-oxo-benzimidazol-1-yl]piperidine-2,6-dione

A solution of tert-butyl 4-[1-(2,6-dioxo-3-piperidyl)-3-methyl-2-oxo-benzimidazol-4-yl]-2,2-dimethyl-piperazine-1-carboxylate (240 mg, 508 umol) in TFA (1 mL) and DCM (6 mL) was stirred at 25° C. for 1 hr. On completion, the reaction mixture was concentrated in vacuo to give the title compound (180 mg, 95% yield, TFA) as yellow oil. LC-MS (ESI+) m/z 372.3 (M+H)⁺.

### 3-[3-Methyl-2-oxo-4-(3-piperazin-1-ylprop-1-ynyl)benzimidazol-1-yl]piperidine-2,6-dione (Intermediate CBD)

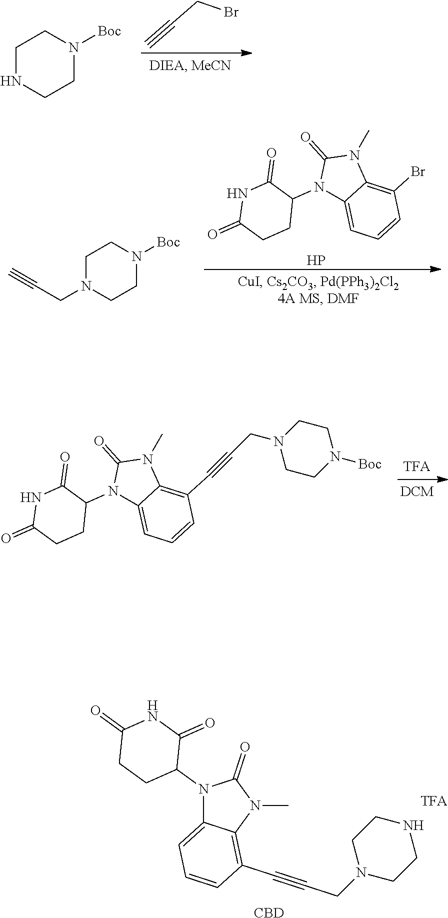

### Step 1—Tert-butyl 4-prop-2-ynylpiperazine-1-carboxylate

To a solution of tert-butyl piperazine-1-carboxylate (1.00 g, 5.37 mmol) in ACN (150 mL) was added DIEA (1.39 g, 10.7 mmol). Next, 3-bromoprop-1-yne (574 mg, 4.83 mmol) was dissolved in ACN (150 mL) and was added dropwise to the reaction mixture. The mixture was stirred at 25° C. for 2 hrs. On completion, the reaction mixture was diluted with H₂O (20 mL) and extracted with EA (20 mL×2). The combined organic layers were concentrated under reduced pressure to give a residue. The residue was purified by column chromatography (SiO₂, Petroleum ether/Ethyl acetate=8/1 to 3/1) to give the title compound (1.00 g, 83% yield) as yellow oil. ¹H NMR (400 MHz, DMSO-d₆) δ 3.32 (s, 4H), 3.28 (d, J=2.4 Hz, 2H), 3.16 (t, J=2.4 Hz, 1H), 2.40-2.34 (m, 4H), 1.39 (s, 9H)

### Step 2—Tert-butyl 4-[3-[1-(2,6-dioxo-3-piperidyl)-3-methyl-2-oxo-benzimidazol-4-yl]prop-2-ynyl]piperazine-1-carboxylate

A mixture of tert-butyl 4-prop-2-ynylpiperazine-1-carboxylate (2 g, 8.92 mmol), 3-(4-bromo-3-methyl-2-oxobenzimidazol-1-yl)piperidine-2,6-dione (2.01 g, 5.94 mmol, Intermediate HP), CuI (56.6 mg, 297 umol), Cs₂CO₃ (5.81 g, 17.8 mmol) and Pd(PPh₃)₂Cl₂ (208 mg, 297 umol) in DMF (15 mL) was stirred at 80° C. for 3 hrs under N₂ atmosphere. On completion, the reaction mixture was filtered and the filtrate was diluted with H₂O (50 mL). Then the mixture was extracted with EA (2×50 mL). The combined organic layer was concentrated in vacuo to give a residue. The residue was purified by reverse phase (0.1% FA condition) to give the title compound (1.6 g, 55.8% yield) as an off-white solid. ¹H NMR (400 MHz, DMSO-d₆) δ 11.12 (s, 1H), 7.19-7.06 (m, 2H), 7.03-6.98 (m, 1H), 5.39 (dd, J=5.2, 12.8 Hz, 1H), 3.63 (s, 3H), 3.60 (s, 2H), 3.39-3.33 (m, 6H), 2.95-2.82 (m, 1H), 2.75 (d, J=4.0 Hz, 1H), 2.66-2.59 (m, 1H), 2.48 (s, 2H), 2.07-1.99 (m, 1H), 1.39 (s, 9H); LC-MS (ESI⁺) m/z 482.3 (M+H)⁺.

### Step 3—3-[3-Methyl-2-oxo-4-(3-piperazin-1-ylprop-1-ynyl)benzimidazol-1-yl]piperidine-2,6-dione

To a solution of tert-butyl 4-[3-[1-(2,6-dioxo-3-piperidyl)-3-methyl-2-oxo-benzimidazol-4-yl]prop-2-ynyl]piperazine-1-carboxylate (70 mg, 145.37 umol) in DCM (1 mL) was added TFA (770 mg, 6.75 mmol). The mixture was then stirred at 25° C. for 1 hr. On completion, the reaction mixture was concentrated in vacuo to give the title compound (65 mg, 90% yield) as a brown solid. LC-MS (ESI+) m/z 382.0 (M+H)⁺.

### 3-Methyl-1-(8-piperazin-1-yl-4-isoquinolyl)hexahydropyrimidine-2,4-dione (Intermediate CBE)

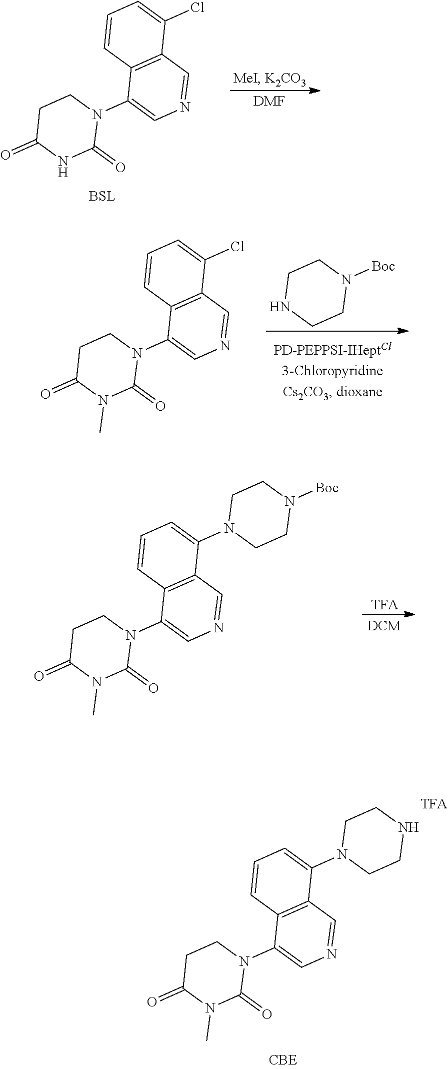

### Step 1—1-(8-Chloro-4-isoquinolyl)-3-methyl-hexahydropyrimidine-2,4-dione

To a solution of 1-(8-chloro-4-isoquinolyl)hexahydropyrimidine-2,4-dione (500 mg, 1.81 mmol, Intermediate BSL) in DMF (5 mL) was added K₂CO₃ (501 mg, 3.63 mmol) and MeI (169 uL, 2.72 mmol), then the mixture was stirred at 60° C. for 6 hours. On completion, the mixture was filtered and the filtrate was extracted with water (50 mL) and EA (50 mL×3). The combined organic layer was dried over anhydrous Na₂SO₄, then concentrated in vacuo to give the residue. The residue was purified by reverse-phase (0.1% FA condition) to give the title compound (170 mg, 32% yield) as a yellow solid. ¹H NMR (400 MHz, DMSO-d₆) δ 9.56 (d, J=0.4 Hz, 1H), 8.71 (s, 1H), 8.06 (d, J=8.4 Hz, 1H), 7.91-7.87 (m, 1H), 7.83-7.77 (m, 1H), 3.91-3.89 (m, 1H), 3.69-3.66 (m, 1H), 3.09 (s, 3H), 3.05-3.02 (m, 1H), 2.95-2.91 (m, 1H). LC-MS (ESI⁺) m/z 290.1 (M+H)⁺.

### Step 2—Tert-butyl 4-[4-(3-methyl-2,4-dioxo-hexahydropyrimidin-1-yl)-8-isoquinolyl]piperazine-1-carboxylate

To a solution of 1-(8-chloro-4-isoquinolyl)-3-methyl-hexahydropyrimidine-2,4-dione (140 mg, 483 umol) and tert-butyl piperazine-1-carboxylate (108 mg, 580 umol, CAS #57260-71-6) in dioxane (5 mL) was added Pd-PEPPSI-IHeptCl₃-Chloropyridine (47.0 mg, 48.3 umol) and Cs₂CO₃ (314 mg, 966 umol), then the mixture was stirred at 80° C. for 8 hours. On completion, the mixture was filtered and the filtrate was concentrated in vacuo to give the residue. The residue was purified by reverse-phase (0.1% FA condition) to give the title compound (106 mg, 50% yield) as a yellow solid. ¹H NMR (400 MHz, DMSO-d₆) δ 9.49 (s, 1H), 8.55 (s, 1H), 7.75-7.66 (m, 2H), 7.32-7.27 (m, 1H), 3.93-3.83 (m, 1H), 3.74-3.58 (m, 5H), 3.46-3.41 (m, 2H), 3.10-3.04 (m, 6H), 2.93-2.86 (m, 1H), 1.45 (s, 8H). LC-MS (ESI⁺) m/z 440.2 (M+H)⁺.

### Step 3—3-Methyl-1-(8-piperazin-1-yl-4-isoquinolyl)hexahydropyrimidine-2,4-dione

To a solution of tert-butyl 4-[4-(3-methyl-2,4-dioxo-hexahydropyrimidin-1-yl)-8-isoquinolyl]piperazine-1-carboxylate (106 mg, 241 umol) in DCM (2 mL) was added TFA (1 mL, 13.5 mmol), then the mixture was stirred at 25° C. for 1 hour. On completion, the mixture was concentrated in vacuo to give the title compound (100 mg, 91% yield, TFA) as a yellow oil. LC-MS (ESI⁺) m/z 340.1 (M+H)⁺.

### 1-(8-bromo-7-methyl-4-isoquinolyl)-3-[(4-methoxyphenyl)methyl]hexahydro pyrimidine-2,4-dione (Intermediate CBF)

### Step 1—8-Bromo-7-methyl-isoquinoline

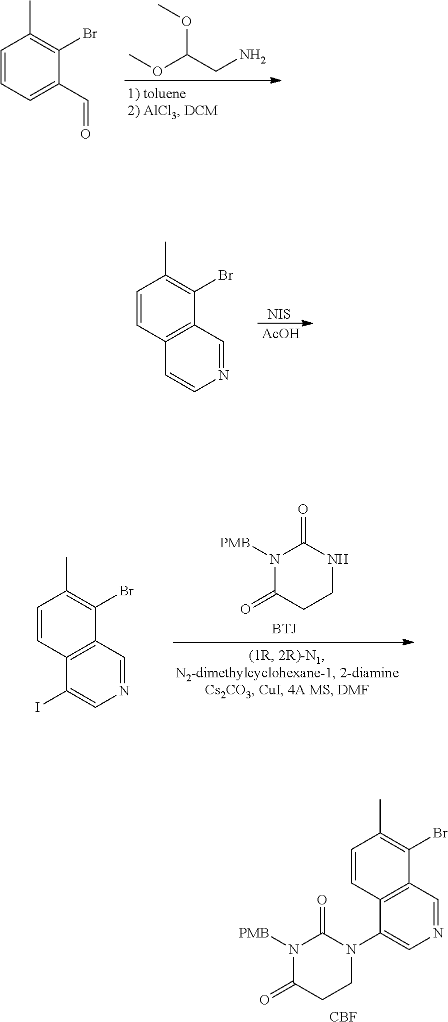

To a solution of 2-bromo-3-methyl-benzaldehyde (500 mg, 2.51 mmol, CAS #109179-31-9) in toluene (8 mL) was added 2,2-dimethoxyethanamine (277 mg, 2.64 mmol, CAS #22483-09-6). The mixture was stirred at 100° C. for 4 hrs. Then the reaction mixture was concentrated in vacuo to give a residue. The residue was dissolved in DCM (15 mL) and cooled to 0° C. Then, AlCl₃ (1.11 g, 8.29 mmol) was added to the mixture and the reaction mixture was stirred at 25° C. for 18 hrs. On completion, the reaction mixture was added into ice water dropwise (80 mL). The reaction mixture was carefully basified with 2 M aqueous NaOH solution (120 mL). The resulting solution was diluted with water (80 mL) and extracted with DCM (2×60 mL). The combined organic layer was dried over Na₂SO₄, filtered and concentrated in vacuo to give a residue. The residue was purified by column chromatography (SiO₂, PE:EA=10:1 to PE:EA=3:1, P1:Rf=0.42) to give the title compound (420 mg, 75% yield) as a yellow solid. ¹H NMR (400 MHz, CDCl₃) δ9.67 (s, 1H), 8.56 (d, J=6.0 Hz, 1H), 7.69 (d, J=8.4 Hz, 1H), 7.59-7.54 (m, 2H), 2.64 (s, 3H); LC-MS (ESI⁺) m/z 222.0 (M+H)⁺.

### Step 2—8-Bromo-4-iodo-7-methyl-isoquinoline

To a solution of 8-bromo-7-methyl-isoquinoline (270 mg, 1.22 mmol) in AcOH (4 mL) was added NIS (601 mg, 2.67 mmol). The reaction mixture was stirred at 100° C. for 12 hrs. On completion, the mixture was concentrated in vacuo to give a residue. The residue was purified by column chromatography (SiO₂, PE:EA=1:0 to PE:EA=50:1, P1:Rf=0.58) to give the title compound (173 mg, 40% yield) as a yellow solid. ¹H NMR (400 MHz, DMSO-d₆) δ 9.44 (s, 1H), 8.98 (s, 1H), 8.01-7.79 (m, 2H), 2.61 (s, 3H); LC-MS (ESI⁺) m/z 349.8 (M+H)⁺.

### Step 3—1-(8-Bromo-7-methyl-4-isoquinolyl)-3-[(4-methoxyphenyl)methyl]hexahydropyrimidine-2,4-dione

To a solution of 8-bromo-4-iodo-7-methyl-isoquinoline (110 mg, 316 umol) and 3-(4-methoxybenzyl)dihydropyrimidine-2,4(1H,3H)-dione (74 mg, 316 umol, Intermediate BTJ) in DMF (3 mL) was added 4 Å molecular sieves (50 mg), Cs₂CO₃ (205 mg, 632 umol), CuI (24.1 mg, 126 umol) and (1R,2R)-N1,N2-dimethylcyclohexane-1,2-diamine (17.9 mg, 126 umol). The reaction mixture was degassed and purged with N₂ three times and the mixture was stirred at 100° C. for 16 hrs under N₂ atmosphere. On completion, the reaction mixture was filtered and concentrated in vacuo to give a residue. The residue was purified by prep-HPLC (column: Phenomenex luna C18 150*25 mm*10 um; mobile phase: [water (0.225% FA)-ACN]; B %: 38%-68%, 10 min) to give the title compound (80 mg, 55% yield) as a white solid. LC-MS (ESI⁺) m/z 456.0 (M+H)⁺.

### 1-(7-Methyl-8-piperazin-1-yl-4-isoquinolyl)hexahydropyrimidine-2,4-dione (Intermediate CBG)

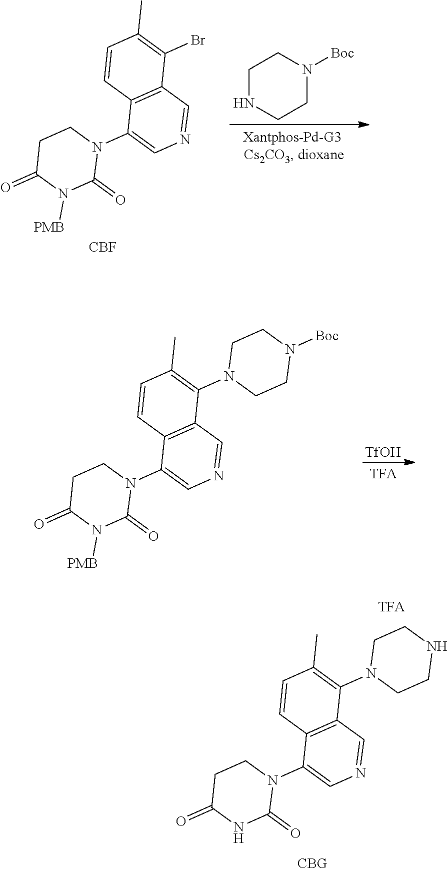

### Step 1—Tert-butyl 4-[4-[3-[(4-methoxyphenyl)methyl]-2,4-dioxo-hexahydropyrimidin-1-yl]-7-methyl-8-isoquinolyl]piperazine-1-carboxylate

To a solution of 1-(8-bromo-7-methyl-4-isoquinolyl)-3-[(4-methoxyphenyl)methyl]hexahydro pyrimidine-2,4-dione (135 mg, 299 umol, Intermediate CBF), and tert-butyl piperazine-1-carboxylate (111 mg, 598 umol, CAS #143238-8-4) in dioxane (5 mL) was added Cs₂CO₃ (292 mg, 897 umol) and XantPhos Pd G3 (28.3 mg, 29.9 umol). Then the reaction mixture was stirred at 110° C. for 16 hrs under N₂. On completion, the reaction mixture was filtered and the filtrate was concentrated in vacuo to give a residue. The residue was purified by prep-HPLC (column: Phenomenex Luna C18 100*30 mm*5 um; mobile phase: [water(FA)-ACN]; B %: 39%-69%, 8 min) to give the title compound (50.0 mg, 30% yield) as an yellow solid. ¹H NMR (400 MHz, DMSO-d₆) δ 9.66 (s, 1H), 8.53 (s, 1H), 7.71-7.58 (m, 2H), 7.25 (d, J=8.8 Hz, 2H), 6.91-6.82 (m, 2H), 4.83 (s, 2H), 3.95-3.87 (m, 1H), 3.85-3.76 (m, 2H), 3.73 (s, 3H), 3.61-3.45 (m, 4H), 3.31-3.21 (m, 2H), 3.18-3.07 (m, 3H), 3.00-2.92 (m, 1H), 2.54-2.50 (m, 2H), 1.46 (s, 9H); LC-MS (ESI⁺) m/z 560.2 (M+H)⁺.

### Step 2—1-(7-Methyl-8-piperazin-1-yl-4-isoquinolyl)hexahydropyrimidine-2,4-dione

To a solution of tert-butyl 4-[4-[3-[(4-methoxyphenyl)methyl]-2,4-dioxo-hexahydropyrimidin-1-yl]-7-methyl-8-isoquinolyl]piperazine-1-carboxylate (50.0 mg, 89.3 umol) in TFA (770 mg, 6.75 mmol, 0.5 mL) was added TfOH (170 mg, 1.13 mmol, 0.1 mL). Then the reaction was stirred at 70° C. for 2 hrs. On completion, the reaction mixture was concentrated in vacuo to give the title compound (40.0 mg, 98% yield, TFA) as yellow oil. LC-MS (ESI⁺) m/z 340.1 (M+H)⁺.

### 1-(8-bromo-7-methoxy-4-isoquinolyl)-3-[(4-methoxyphenyl)methyl]hexahydropyrimidine-2,4-dione (Intermediate CBH)

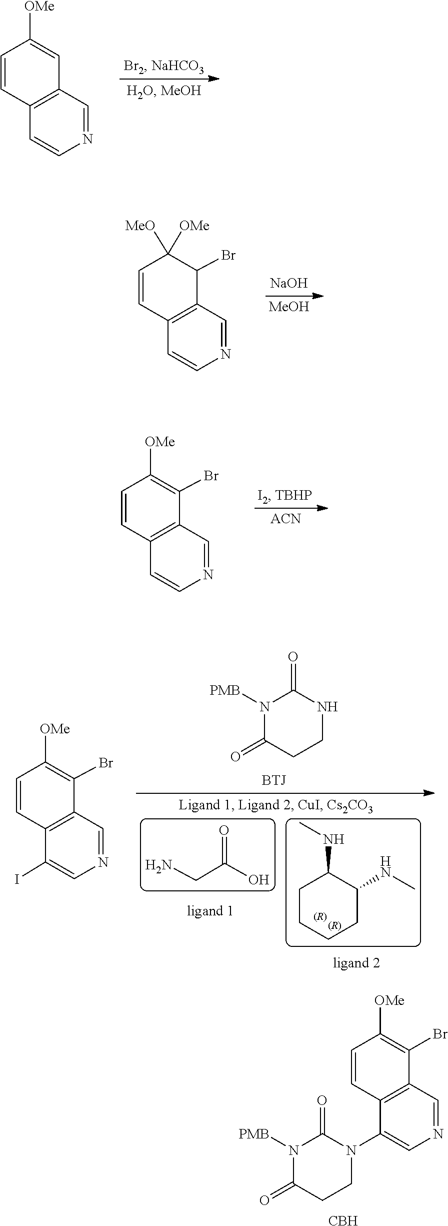

### Step 1—8-bromo-7,7-dimethoxy-8H-isoquinoline

To a solution of 7-methoxyisoquinoline (3.6 g, 23 mmol) in MeOH (70 mL) was added NaHCO₃ (3.32 g, 40 mmol, 1.54 mL) and Br₂ (5.42 g, 34 mmol, 1.75 mL). The mixture was stirred at 25° C. for 5 mins. Then H₂O (30 mL) was added into above solution, and the mixture was stirred at 25° C. for 5 mins. On completion, the mixture was quenched by Na₂SO₃ and H₂O (50 mL), then extracted with DCM (3×30 mL). The combined organic layer was washed with brine (3×30 mL), dried with anhydrous Na₂SO₄, then filtered and the liquor was concentrated in vacuo to give the title compound (4.4 g, 17 mmol, 72% yield) as a brown solid. LC-MS (ESI⁺) m/z 270.0 (M+H)⁺.

### Step 2—8-bromo-7-methoxy-isoquinoline

To a solution of 8-bromo-7,7-dimethoxy-8H-isoquinoline (4.4 g, 17 mmol) in MeOH (80 mL) was added NaOH (3.26 g, 82 mmol). The mixture was stirred at 25° C. for 10 mins. On completion, the mixture was diluted with H₂O(50 mL), then extracted with DCM (3×100 mL). The combined organic layer was washed with brine (3×100 mL), dried with anhydrous Na₂SO₄, then filtered and the liquor was concentrated in vacuo and the residue was purified by column chromatography (SiO₂, Petroleum ether/Ethyl acetate=100/1 to 5/1, PE:EA=1:1, Rf=0.50) to give the title compound (2.8 g, 72% yield) as a white solid. ¹H NMR (400 MHz, CDCl₃-d) 69.64 (s, 1H), 8.50 (d, J=5.6 Hz, 1H), 7.84 (d, J=9.0 Hz, 1H), 7.60 (d, J=5.6 Hz, 1H), 7.50 (d, J=9.0 Hz, 1H), 4.07 (s, 3H). LC-MS (ESI⁺) m/z 238.0 (M+H)⁺.

### Step 3—8-bromo-4-iodo-7-methoxy-isoquinoline

To a solution of 8-bromo-7-methoxy-isoquinoline (1.84 g, 7.73 mmol) in ACN (120 mL) was added I₂ (2.35 g, 9.27 mmol, 1.87 mL) and TBHP (7.96 g, 61.8 mmol, 8.47 mL, 70% solution) at 25° C. The mixture was then stirred at 80° C. for 16 hrs. On completion, the mixture was quenched by Na₂S₂O₃—H₂O (100 mL), diluted with H₂O (50 mL), then extracted with EA (3×100 mL). The combined organic layer was washed with brine (3×100 mL), dried with anhydrous Na₂SO₄. The mixture was filtered and the liquor was concentrated in vacuo, the residue was purified by column chromatography (SiO₂, Petroleum ether/Ethyl acetate=50/1 to 3/1)(PE:EA=1:1, Rf=0.7) to give the title compound (1.12 g, 40% yield) as a yellow solid. 1H NMR (400 MHz, CDCl₃-d) 69.56 (s, 1H), 8.88 (s, 1H), 8.07 (d, J=9.4 Hz, 1H), 7.58 (d, J=9.4 Hz, 1H), 4.10 (s, 3H). LC-MS (ESI⁺) m/z 363.8 (M+H)⁺.

### Step 4—1-(8-bromo-7-methoxy-4-isoquinolyl)-3-[(4-methoxyphenyl)methyl]hexahydropyrimidine-2,4-dione

To a solution of 8-bromo-4-iodo-7-methoxy-isoquinoline (300 mg, 824 umol) and 3-[(4-methoxyphenyl)methyl]hexahydropyrimidine-2,4-dione (193.08 mg, 824 umol, Intermediate BTJ) in DMF (15 mL) was added 4 Å molecular sieves (375 mg), Cs₂CO₃ (537.10 mg, 1.65 mmol), CuI (62.79 mg, 329.7 umol), 2-aminoacetic acid (24.75 mg, 329.7 umol) and (1R,2R)-N1,N2-dimethylcyclohexane-1,2-diamine (46.90 mg, 329.7 umol). The mixture was then stirred at 100° C. for 16 hrs. On completion, the mixture was filtered and the liquor was concentrated in vacuo. The crude product was purified by reversed-phase HPLC(0.1% FA condition) to give the title compound (95 mg, 25% yield) as a yellow solid. ¹H NMR (400 MHz, CHLOROFORM-d) 67.58 (br s, 1H), 7.56 (br s, 1H), 7.44 (d, J=8.6 Hz, 4H), 6.88 (s, 2H), 5.00 (d, J=3.6 Hz, 2H), 3.82 (s, 3H), 3.76-3.68 (m, 2H), 3.04 (br s, 2H), 2.06 (s, 1H). LC-MS (ESI⁺) m/z 471.9 (M+H)⁺.

### 1-(7-Methoxy-8-piperazin-1-yl-4-isoquinolyl)hexahydropyrimidine-2,4-dione (Intermediate CBI)

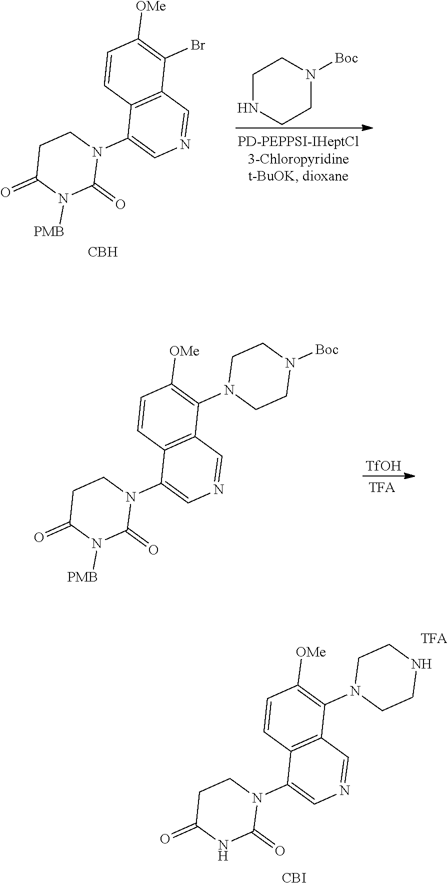

### Step 1—Tert-butyl 4-[7-methoxy-4-[3-[(4-methoxyphenyl)methyl]-2,4-dioxo-hexahydropyrimidin-1-yl]-8-isoquinolyl]piperazine-1-carboxylate

To a solution of 1-(8-bromo-7-methoxy-4-isoquinolyl)-3-[(4-methoxyphenyl)methyl]hexahydropyrimidine-2,4-dione (100 mg, 212 umol, Intermediate CBH) and tert-butyl piperazine-1-carboxylate (47.5 mg, 255 umol, CAS #143238-38-4) in DMA (2 mL) was added RuPhos Pd G3 (17.7 mg, 21.2 umol) and Cs₂CO₃ (138 mg, 425 umol). Then the mixture was stirred at 110° C. for 10 hours. On completion, the mixture was filtered and concentrated in vacuo to give the residue. The residue was purified by reverse-phase (0.1% FA condition) to give the title compound (50.0 mg, 20% yield) as yellow solid. ¹HNMR (400 MHz, DMSO-d₆) δ 9.69 (s, 1H), 8.42 (s, 1H), 8.10 (d, J=7.6 Hz, 2H), 7.81-7.76 (m, 1H), 7.72-7.67 (m, 1H), 7.47 (d, J=8.0 Hz, 2H), 7.25 (d, J=8.4 Hz, 2H), 6.88 (d, J=8.4 Hz, 2H), 4.83 (s, 2H), 3.96 (s, 3H), 3.73 (s, 3H), 3.30 (br s, 2H), 3.18-3.06 (m, 3H), 3.00-2.81 (m, 4H), 2.52 (br s, 3H), 1.45 (s, 9H); LC-MS (ESI⁺) m/z 576.2 (M+H)⁺.

### Step 2—1-(7-Methoxy-8-piperazin-1-yl-4-isoquinolyl)hexahydropyrimidine-2,4-dione

To a solution of tert-butyl 4-[7-methoxy-4-[3-[(4-methoxyphenyl)methyl]-2,4-dioxo-hexahydropyrimidin-1-yl]-8-isoquinolyl]piperazine-1-carboxylate (30.0 mg, 52.1 umol) in TFA (1 mL) was added TfOH (0.2 mL, 2.27 mmol), then the mixture was stirred at 70° C. for 30 minutes. On completion, the mixture was concentrated in vacuo to give the title compound (24.0 mg, 98% yield, TFA) as yellow oil. LC-MS (ESI⁺) m/z 356.1 (M+H)⁺.

### 6-(1-Fluoro-1-methyl-ethyl)pyridine-2-carboxylic acid (Intermediate CBJ)

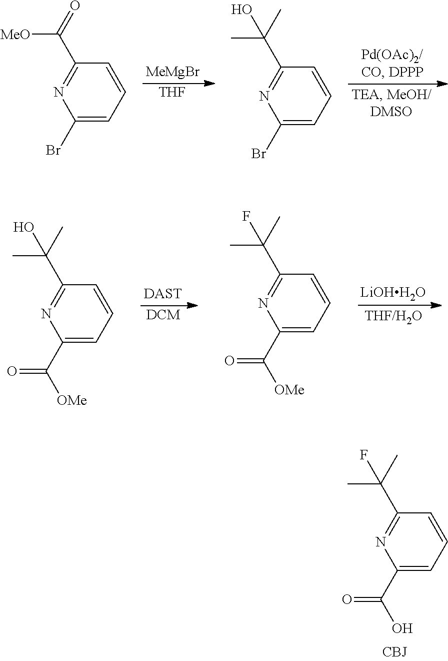

### Step 1—2-(6-Bromo-2-pyridyl)propan-2-ol

To a solution of methyl 6-bromopyridine-2-carboxylate (10 g, 46.2 mmol, CAS #26218-75-7) in THF (100 mL) was added MeMgBr (3 M, 77.1 mL, CAS #75-16-1) at 0° C. under N₂. The mixture was stirred at 25° C. for 1 hour. On completion, the reaction was quenched with NH₄Cl (200 mL, aq.), then extracted with EA (150 mL×3). The combined organic layer was washed with NaHCO₃ (100 mL×3, aq.) and brine (100 mL), dried over Na₂SO₄, filtered and concentrated in vacuo to afford the title compound (5 g, 50% yield) as a white solid. LC-MS (ESI⁺) m/z 215.8 (M+H)⁺.

### Step 2—Methyl 6-(1-hydroxy-1-methyl-ethyl)pyridine-2-carboxylate

To a solution of 2-(6-bromo-2-pyridyl)propan-2-ol (3.5 g, 16.2 mmol) in MeOH (35 mL) and DMSO (35 mL) were added DPPP (668 mg, 1.62 mmol), Pd(OAc)₂ (363 mg, 1.62 mmol) and TEA (4.10 g, 40.5 mmol, 5.64 mL). The mixture was degassed and purged with CO (16.2 mmol) three times, then the mixture was stirred at 25° C. for 30 minutes. Next, the solution was warmed up to 80° C. and stirred under CO at 40 psi for 16 hours. On completion, the reaction was filtered and the filtrate was diluted with water (150 mL), and extracted with EA (50 mL×3). The organice phase was then washed with brine (30 mL×2), dried over Na₂SO₄, filtered and concentrated under reduced pressure to give the desired product. Then the crude was purified by column chromatography (SiO₂, Petroleum ether/Ethyl acetate=100/1 to 10/1) to afford the title compound (3 g, 95% yield) as a white solid. ¹H NMR (400 MHz, CDCl₃) δ 8.02 (d, J=7.6 Hz, 1H), 7.86 (t, J=8.0 Hz, 1H), 7.57 (d, J=8.0 Hz, 1H), 5.37-4.52 (m, 1H), 3.99 (s, 3H), 1.58 (s, 6H). LC-MS (ESI⁺) m/z 196.3 (M+H)⁺.

### Step 3—Methyl 6-(1-fluoro-1-methyl-ethyl)pyridine-2-carboxylate

To a solution of methyl-6-(1-hydroxy-1-methyl-ethyl) pyridine-2-carboxylate (1 g, 5.12 mmol) in DCM (20 mL) was added DAST (1.01 g, 5.63 mmol, 90% solution) at 0° C. Then the mixture was stirred at 25° C. for 16 hours. On completion, the reaction was diluted with water (100 mL), and extracted with EA (30 mL×3). The combined organic layers were washed with brine (20 mL×2), dried over Na₂SO₄, filtered and concentrated under reduced pressure to give a residue. Then the residue was purified by column chromatography (SiO₂, Petroleum ether/Ethyl acetate=100/1 to 20/1) to afford the title compound (2 g, 48% yield) as a white solid. LC-MS (ESI⁺) m/z 198.3 (M+H)⁺.

### Step 4—6-(1-Fluoro-1-methyl-ethyl)pyridine-2-carboxylic acid

To a solution of methyl 6-(1-fluoro-1-methyl-ethyl)pyridine-2-carboxylate (1.9 g, 9.63 mmol) in THF (12 mL) and H₂O (4 mL) was added LiOH·H₂O (808 mg, 19.3 mmol) at 0° C. The mixture was then stirred at 25° C. for 3 hours. On completion, the organic solvent was removed in vacuo and the crude product was purified by reversed-phase HPLC (0.1% FA) to give the title compound (1.7 g, 90% yield) as a yellow solid. LC-MS (ESI⁺) m/z 184.1 (M+H)⁺.

### 6-(1-fluoro-1-methyl-ethyl)-N-[2-(4-formylcyclohexyl)-6-methoxy-indazol-5-yl]-N-methyl-pyridine-2-carboxamide (Intermediate CBK)

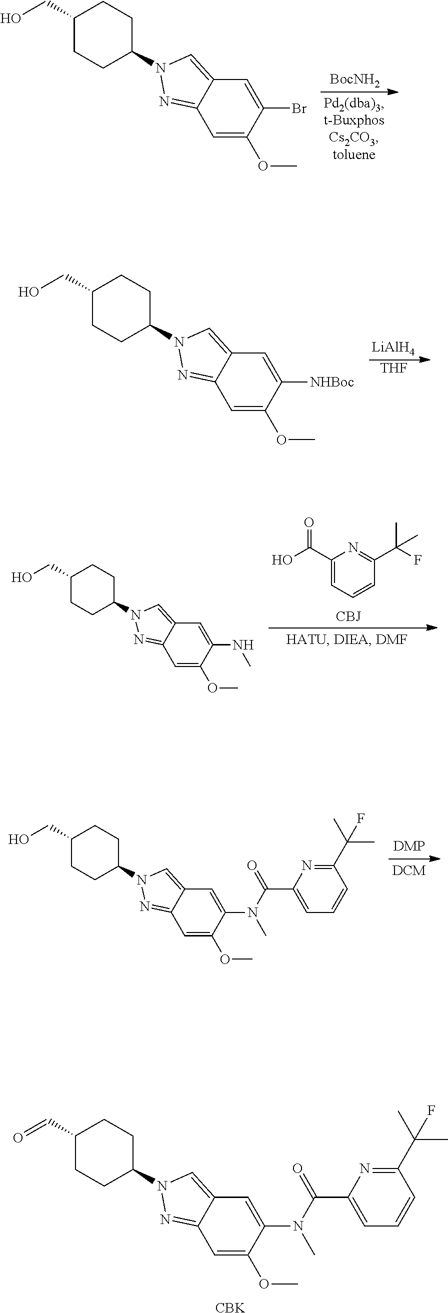

### Step 1—[4-[6-methoxy-5-(methylamino)indazol-2-yl]cyclohexyl]methanol

To a solution of tert-butyl N-[2-[4-(hydroxymethyl) cyclohexyl]-6-methoxy-indazol-5-yl]carbamate (1 g, 2.66 mmol, synthesized via Step 1 of Intermediate BGT) in THF (30 mL) was added LiAlH₄ (505 mg, 13.3 mmol) at 0° C. After 10 minutes, the cooling bath was removed and after stirring at 25° C. for 20 minutes, the mixture was stirred at 60° C. for 16 hours under N2 atmosphere. On completion, the reaction mixture was quenched with H₂O (1 mL) at 0° C., and then diluted with NaOH (1 M, 1 mL, aq.) and dried over Na2SO4, filtered and concentrated under reduced pressure to give a residue. The residue was purified by prep-HPLC (column: YMC Triart C18 250*50 mm*7 um; mobile phase: [water (HCl)-ACN]; B %: 5%-35%, 23 minutes) to afford the title compound (400 mg, 44% yield) as a yellow solid. ¹H NMR (400 MHz, DMSO-d₆) δ 8.47 (s, 1H), 7.83 (d, J=7.2 Hz, 1H), 7.23 (s, 1H), 4.48-4.34 (m, 2H), 3.94 (s, 3H), 3.28 (d, J=6.4 Hz, 2H), 2.87 (s, 4H), 2.12 (d, J=8.0 Hz, 2H), 1.92-1.86 (m, 4H), 1.53-1.41 (m, 1H), 1.19-1.09 (m, 2H). LC-MS (ESI⁺) m/z 290.3 (M+H)⁺.

### Step 2—6-(1-fluoro-1-methyl-ethyl)-N-[2-[4-(hydroxymethyl)cyclohexyl]-6-methoxy-indazol-5-yl]-N-methyl-pyridine-2-carboxamide

To a solution of [4-[6-methoxy-5-(methylamino)indazol-2-yl]cyclohexyl]methanol (400 mg, 1.38 mmol) in DMF (4 mL) and HATU (788 mg, 2.07 mmol) was added dropwise DIEA (535 mg, 4.15 mmol, 722 uL) until the pH=8 at 0° C. After addition, the mixture was stirred at this temperature for 30 minutes, and then 6-(1-fluoro-1-methyl-ethyl) pyridine-2-carboxylic acid (278 mg, 1.52 mmol, Intermediate CBJ) in DMF (4 mL) was added dropwise at 0° C. The resulting mixture was then stirred at 25° C. for 2 hours. On completion, the reaction mixture was quenched with H₂O (1 mL), the residue was diluted with H₂O (10 mL) and extracted with EA (8 mL×3). The combined organic layers were washed with brine (5 mL×2), dried over Na₂SO₄, filtered and concentrated under reduced pressure to give a residue. The residue was purified by reversed-phase (0.1% FA condition) to give the title compound (320 mg, 47% yield) as yellow oil. ¹H NMR (400 MHz, DMSO-d₆) δ 8.15 (s, 1H), 7.78 (s, 1H), 7.52 (d, J=8.0 Hz, 1H), 7.33 (s, 1H), 7.28 (d, J=7.6 Hz, 1H), 6.89 (s, 1H), 4.46 (s, 1H), 4.34-4.23 (m, 1H), 3.71 (s, 3H), 3.27 (s, 4H), 2.03 (d, J=6.4 Hz, 2H), 1.89-1.77 (m, 4H), 1.43 (d, J=8.2 Hz, 1H), 1.24-0.96 (m, 9H). LC-MS (ESI⁺) m/z 455.3 (M+H)⁺.

### Step 3—6-(1-fluoro-1-methyl-ethyl)-N-[2-(4-formylcyclohexyl)-6-methoxy-indazol-5-yl]-N-methyl-pyridine-2-carboxamide

To a solution of 6-(1-fluoro-1-methyl-ethyl)-N-[2-[4-(hydroxymethyl)cyclohexyl]-6-methoxy-indazol-5-yl]-N-methyl-pyridine-2-carboxamide (200 mg, 440 umol) in DCM (2 mL) was added DMP (279 mg, 660 umol). Then the mixture was stirred at 25° C. for 3 hours. On completion, the reaction mixture was quenched with Na₂S₂O₃ (2 mL, aq.) at 25° C., and then diluted with NaHCO₃ (60 mL, aq.) and extracted with DCM (30 mL×3). The combined organic layers were washed with brine (50 mL), dried over Na₂SO₄, filtered and concentrated under reduced pressure to give the title compound (200 mg, 90% yield) as a yellow oil. LC-MS (ESI⁺) m/z 453.2 (M+H)⁺.

### 3-[4-(azetidin-3-yl)-3-methyl-2-oxo-benzimidazol-1-yl]piperidine-2,6-dione (Intermediate CBL)

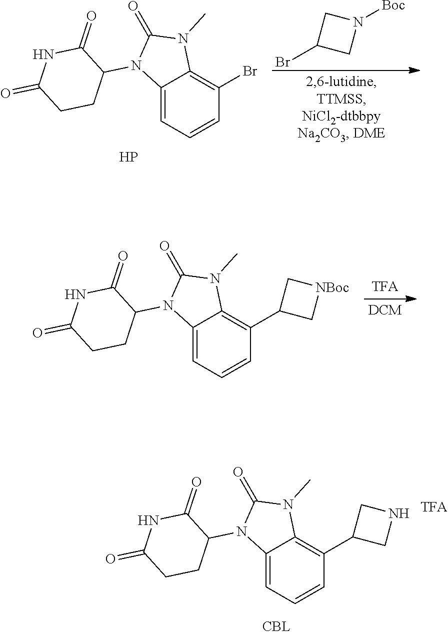

### Step 1—Tert-butyl 3-[1-(2,6-dioxo-3-piperidyl)-3-methyl-2-oxo-benzimidazol-4-yl]azetidine-1-carboxylate

To a solution of 3-(4-bromo-3-methyl-2-oxo-benzimidazol-1-yl)piperidine-2,6-dione (3.5 g, 10.3 mmol, Intermediate HP) in DME (5 mL) was added tert-butyl 3-bromoazetidine-1-carboxylate (3.18 g, 13.4 mmol, CAS #106419-10-0), Ir[dF(CF3)ppy]2(dtbpy)(PF6) (232 mg, 207 umol), NiCl2-dtbbpy (123 mg, 310 umol) 2,6-lutidine (2.22 g, 20.7 mmol, 2.41 mL) and TTMSS (2.57 g, 10.3 mmol, 3.19 mL). The mixture was then stirred at 25° C. for 14 hours. On completion, the reaction mixture was filtered and concentrated under reduced pressure to give a residue. The residue was purified by column chromatography (SiO₂, DCM/Ethyl acetate=100/1 to 20/1) to give the title compound (2.2 g, 50% yield) as a white solid. ¹H NMR (400 MHz, DMSO-d₆) δ 11.09 (s, 1H), 7.24 (d, J=8.0 Hz, 1H), 7.14-7.01 (m, 2H), 5.41-5.34 (m, 1H), 4.49 (t, J=6.4 Hz, 1H), 4.28 (t, J=8.0 Hz, 2H), 3.96 (s, 2H), 3.50 (s, 3H), 2.93-2.83 (m, 1H), 2.06-1.92 (m, 1H), 1.41 (s, 9H), 1.04 (d, J=6.4 Hz, 2H). LC-MS (ESI⁺) m/z 359.0 (M+H)⁺.

### Step 2—3-[4-(azetidin-3-yl)-3-methyl-2-oxo-benzimidazol-1-yl]piperidine-2,6-dione

To a solution of tert-butyl 3-[1-(2,6-dioxo-3-piperidyl)-3-methyl-2-oxo-benzimidazol-4-yl]azetidine-1-carboxylate (500 mg, 1.21 mmol) in DCM (3 mL) was added TFA (1.54 g, 13.5 mmol, 1 mL). The mixture was then stirred at 25° C. for 3 hours. On completion, the reaction was concentrated under reduced pressure to give the title compound (300 mg, 76% yield, TFA salt) as a colorless oil. LC-MS (ESI⁺) m/z 315.1 (M+H)⁺.

### 2-(2,6-dioxo-3-piperidyl)-4-[7-(methylamino)heptylamino]isoindoline-1,3-dione (Intermediate CBM)

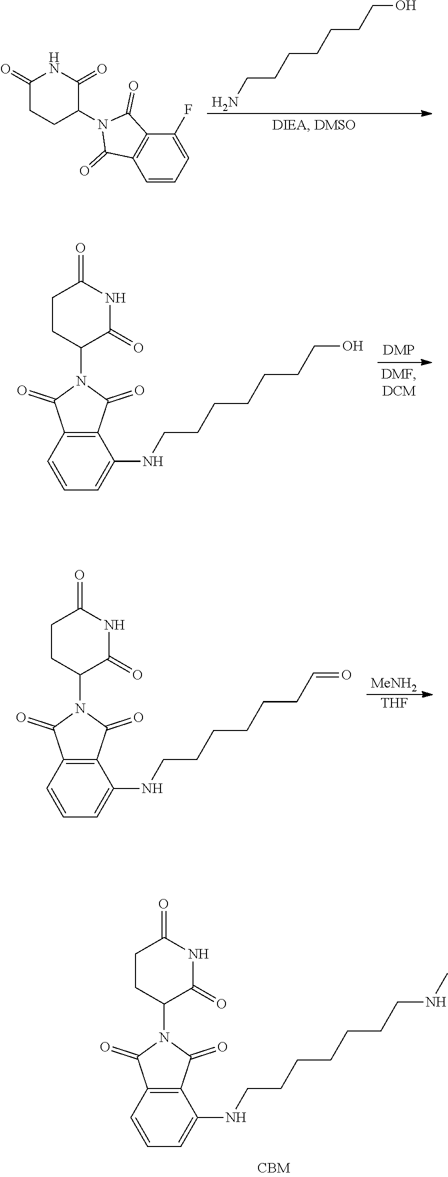

### Step 1—2-(2,6-dioxo-3-piperidyl)-4-(7-hydroxyheptylamino)isoindoline-1,3-dione

A mixture of 2-(2,6-dioxo-3-piperidyl)-4-fluoro-isoindoline-1,3-dione (200 mg, 724 umol, CAS #835616-60-9), 7-aminoheptan-1-ol (114 mg, 868 umol, CAS #19243-04-0), DIEA (280 mg, 2.17 mmol) in DMSO (2 mL) was stirred at 130° C. for 1 hour. On completion, the reaction mixture was partitioned between ethyl acetate (1 mL×3) and water (2 mL). The combined organic phase was dried over anhydrous sodium sulfate, filtered and concentrated to give the title compound (230 mg, 70% yield) LC-MS (ESI+) m/z 388.2 (M+H)⁺.

### Step 2—7-[[2-(2,6-dioxo-3-piperidyl)-1,3-dioxo-isoindolin-4-yl]amino]heptanal

To a solution of 2-(2,6-dioxo-3-piperidyl)-4-(7-hydroxyheptylamino)isoindoline-1,3-dione (200 mg, 516 umol) in DMF (1 mL), DCM (1 mL) was added DMP (284 mg, 671 umol). The mixture was stirred at 25° C. for 1 hour. On completion, the reaction mixture was quenched with water (10 mL) at 20° C., and then diluted with sodium thiosulfate (4 mL) and extracted with DCM (2 mL×3). The combined organic layers were washed with NaHCO₃ 1 mL (1 mL×3), dried over anhydrous sodium sulfate, filtered and concentrated under reduced pressure to give the title compound (240 mg, 80% yield). LC-MS (ESI+) m/z 386.1 (M+H)⁺.

### Step 3—2-(2,6-dioxo-3-piperidyl)-4-[7-(methylamino)heptylamino]isoindoline-1,3-dione

To a solution of 7-[[2-(2,6-dioxo-3-piperidyl)-1,3-dioxo-isoindolin-4-yl]amino]heptanal (240 mg, 622 umol) in THF (1 mL) was added methanamine; hydrochloride (50.4 mg, 747 umol) and NaBH(OAc)₃ (329 mg, 1.56 mmol). The mixture was stirred at 25° C. for 0.5 hour. On completion, the residue was purified by reverse phase to give the title compound (40.0 mg, 70% yield). LC-MS (ESI+) m/z 401.2 (M+H)⁺.

### N-[2-(4-formylcyclohexyl)-6-(1-hydroxy-1-methyl-ethyl)indazol-5-yl]-6-(trifluoromethyl)pyridine-2-carboxamide (Intermediate CBN)

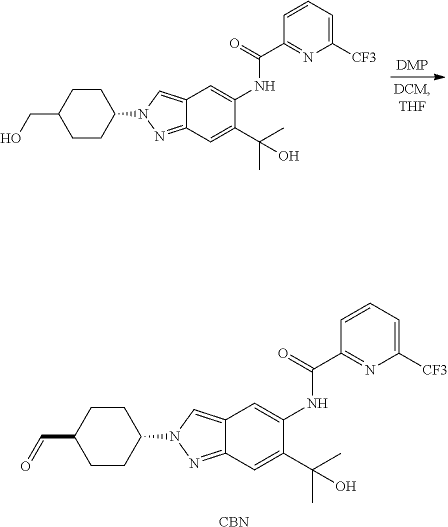

To a solution of N-[2-[4-(hydroxymethyl)cyclohexyl]-6-(1-hydroxy-1-methyl-ethyl)indazol-5-yl]-6-(trifluoromethyl)pyridine-2-carboxamide (100 mg, 209 umol, synthesized via Steps 1-2 of Intermediate AGL) in DCM (0.5 mL) THF (0.5 mL) was added DMP (115 mg, 272 umol). The mixture was then stirred at 25° C. for 20 min. On completion, the reaction mixture was partitioned between ethyl acetate (3 mL×3) and water (5 mL). The combined organic phase was dried over anhydrous sodium sulfate, filtered and concentrated to give the title compound (105 mg, 90% yield). LC-MS (ESI+) m/z 474.9 (M+H)⁺.

### 3-[4-(7-azaspiro[3.5]nonan-2-ylamino)-1-oxo-isoindolin-2-yl]piperidine-2,6-dione (Intermediate CBO)

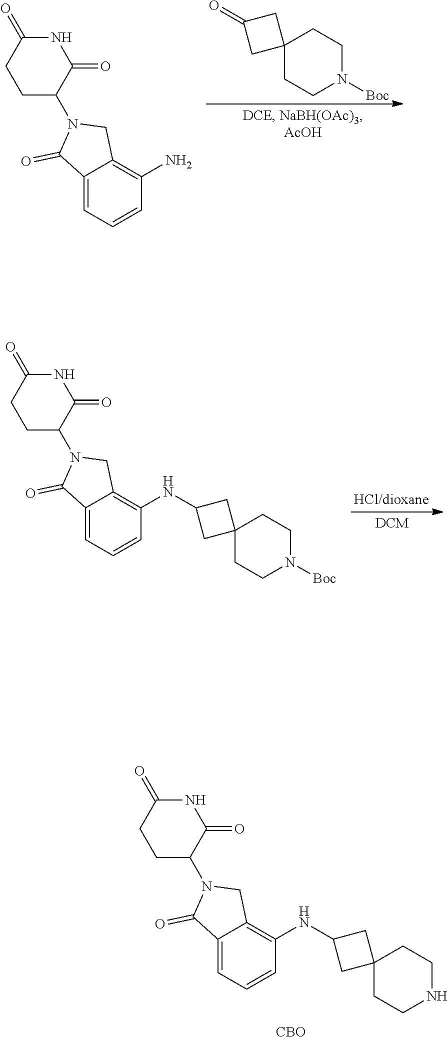

### Step 1—Tert-butyl 2-[[2-(2,6-dioxo-3-piperidyl)-1-oxo-isoindolin-4-yl]amino]-7-azaspiro[3.5]nonane-7-carboxylate

To a solution of 3-(4-amino-1-oxo-isoindolin-2-yl) piperidine-2,6-dione (500 mg, 1.93 mmol, CAS #191732-72-6) and tert-butyl 2-oxo-7-azaspiro[3.5]nonane-7-carboxylate (507 mg, 2.12 mmol, CAS #203661-69-2) in DCE (10.0 mL) was added NaBH(OAc)₃ (531 mg, 2.51 mmol) and AcOH (1.97 g, 32.7 mmol, 1.8 mL). The mixture was then stirred at 25° C. for 2 hours. On completion, the mixture was filtered and concentrated under reduced pressure to give a residue. The crude product was triturated with dichloromethane:acetonitrile (1:10, 80.0 mL) at 25° C. for 30 minutes to give the title compound (670 mg, 71% yield). LC-MS (ESI⁺) m/z 483.2 (M+H)+

### Step 2—3-[4-(7-Azaspiro [3.5]nonan-2-ylamino)-1-oxo-isoindolin-2-yl]piperidine-2, 6-dione

To a solution of tert-butyl 2-[[2-(2,6-dioxo-3-piperidyl)-1-oxo-isoindolin-4-yl]amino]-7-azaspiro[3.5]nonane-7-carboxylate (120 mg, 248 umol) in DCM (3 mL) was added HCl/dioxane (4 M, 1.2 mL). The mixture was then stirred at 25° C. for 30 min. On completion, the reaction mixture was concentrated under reduced pressure to remove solvent to give the title compound (100 mg, 95% yield) as a white solid. LC-MS (ESI⁺) m/z 383.1 (M+H)⁺.

### 2-(2,6-dioxo-3-piperidyl)-4-[3-(methylamino)propylamino]isoindoline-1,3-dione (Intermediate CBP)

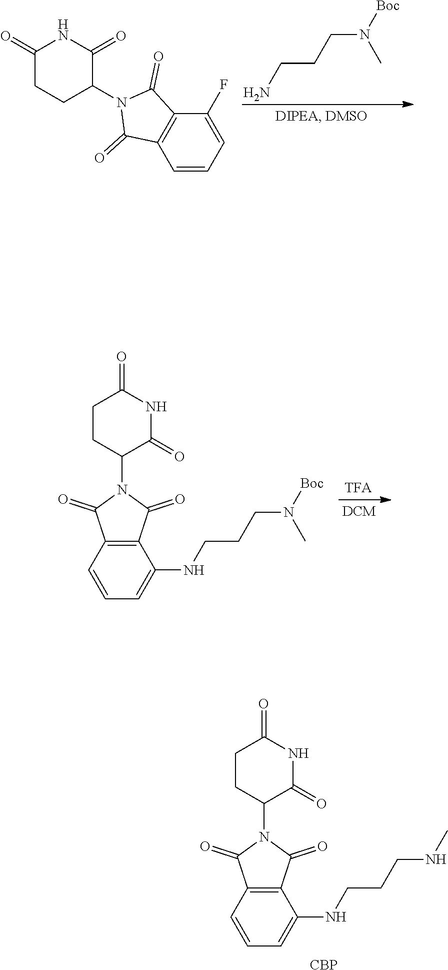

### Step 1—Tert-butyl N-[3-[[2-(2,6-dioxo-3-piperidyl)-1,3-dioxo-isoindolin-4-yl]amino]propyl]-N -methyl-carbamate

To a solution of 2-(2,6-dioxo-3-piperidyl)-4-fluoro-isoindoline-1,3-dione (150 mg, 543 umol, CAS #835616-60-9), and tert-butyl N-(3-aminopropyl)-N-methyl-carbamate (122 mg, 651 umol, CAS #150349-30-3) in DMSO (2 mL) was added DIEA (210 mg, 1.63 mmol). The mixture was then stirred at 130° C. for 1 hours. On completion, the mixture was filtered by H₂O and concentrated in vacuo to give the title compound (200 mg, 80% yield) as green solid. LC-MS (ESI⁺) m/z 345.1 (M+H)⁻¹⁰⁰.

### Step 2—2-(2,6-Dioxo-3-piperidyl)-4-[3-(methylamino)propylamino]isoindoline-1,3-dione

To a solution of tert-butyl N-[3-[[2-(2,6-dioxo-3-piperidyl)-1,3-dioxo-isoindolin-4-yl]amino]propyl]-N -methyl-carbamate (200 mg, 449 umol) in DCM (1 mL) was added TFA (1.54 g, 13.5 mmol, 1 mL). The mixture was then stirred at 25° C. for 5 min. On completion, the reaction mixture was concentrated in vacuo to give the title compound (160 mg, 95% yield) as green solid. LC-MS (ESI⁺) m/z 345.0 (M+H)⁺.

### 4-(4-Aminobutylamino)-2-(2,6-dioxo-3-piperidyl)isoindoline-1,3-dione (Intermediate CBQ)

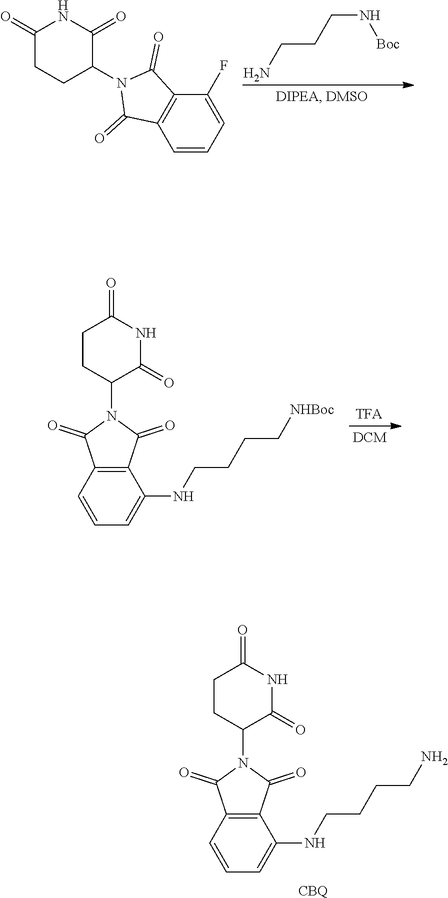

### Step 1—Tert-butyl N-[4-[[2-(2,6-dioxo-3-piperidyl)-1,3-dioxo-isoindolin-4-yl]amino]butyl]carbamate

To a solution of 2-(2,6-dioxo-3-piperidyl)-4-fluoro-isoindoline-1,3-dione (200 mg, 724 umol CAS #835616-60-9) tert-butyl N-(4-aminobutyl)carbamate (163 mg, 868 umol, CAS #68076-36-8) in DMSO (3.0 mL) was added DIEA (280 mg, 2.17 mmol). The mixture was stirred at 130° C. for 2 hours. On completion, the mixture was filtered and concentrated to give the title compound (250 mg, 77% yield) as a yellow solid. ¹H NMR (400 MHz, DMSO-d₆) δ 11.34-10.33 (m, 1H), 7.58 (t, J=7.6 Hz, 1H), 7.11 (d, J=8.4 Hz, 1H), 7.02 (d, J=7.2 Hz, 1H), 5.05 (dd, J=5.6, 12.8 Hz, 1H), 3.31-3.27 (m, 2H), 3.00-2.89 (m, 4H), 2.02 (dd, J=5.2, 10.4 Hz, 1H), 1.59-1.42 (m, 6H), 1.37 (s, 9H), 1.36 (s, 2H).

### Step 2—4-(4-aminobutylamino)-2-(2,6-dioxo-3-piperidyl)isoindoline-1,3-dione

To a solution of tert-butyl N-[4-[[2-(2,6-dioxo-3-piperidyl)-1,3-dioxo-isoindolin-4-yl]amino]butyl]carbamate (150 mg, 337 umol) in DCM (1 mL) was added TFA (1.54 g, 13.5 mmol). The mixture was stirred at 20° C. for 0.5 hours. On completion, the mixture was filtered and concentrated to give the title compound (116 mg, 95% yield) as a yellow solid. LC-MS (ESI+) m/z 345.1 (M+H)⁺.

### N-[2-[4-(aminomethyl)cyclohexyl]-6-(1-hydroxy-1-methyl-ethyl)indazol-5-yl]-6-(trifluoromethyl)pyridine-2-carboxamide (Intermediate CBR)

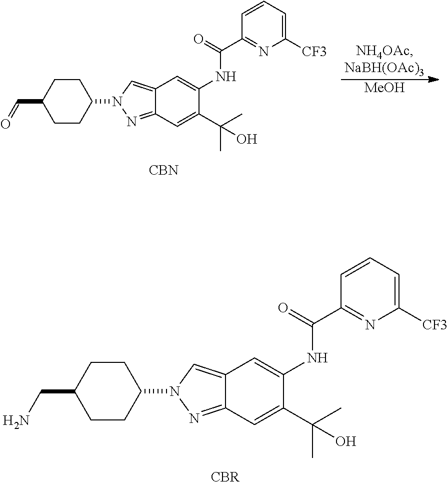 [SMILES: "MeOH" ; CC(C)(O)c1cc2nn([C@H]3CC[C@H](C=O)CC3)cc2cc1NC(=O)c1cccc(C(F)(F)F)n1 ; "CBN" ; CC(C)(O)c1cc2nn([C@H]3CC[C@H](CN)CC3)cc2cc1NC(=O)c1cccc(C(F)(F)F)n1 ; "CBR"]

A mixture of N-[2-(4-formylcyclohexyl)-6-(1-hydroxy-1-methyl-ethyl)indazol-5-yl]-6-(trifluoromethyl)pyridine-2-carboxamide (100 mg, 210 umol, Intermediate CBN), NH₄OAc (324 mg, 4.22 mmol), NaBH₃CN (19.8 mg, 316 umol) in MeOH (1 mL) was degassed and purged with N₂ three times. Then the mixture was stirred at 25° C. for 12 hours. On completion, the crude product was purified by reversed-phase HPLC (0.1% FA condition) to give the title compound (80 mg, 70% yield) as brown liquid. ¹H NMR (400 MHz, DMSO-d6) δ 12.37 (s, 1H), 8.72 (s, 1H), 8.50-8.42 (m, 1H), 8.41-8.31 (m, 3H), 8.17 (dd, J=0.9, 7.6 Hz, 1H), 7.58 (s, 1H), 4.50-4.37 (m, 1H), 3.51-3.49 (m, 2H), 2.16 (d, J=10.8 Hz, 3H), 2.01-1.84 (m, 5H), 1.63 (s, 6H), 1.29-1.12 (m, 3H). LC-MS (ESI⁺) m/z 476.1 (M+H)⁺.

### Tert-butyl N-[3-(4-aminocyclohexoxy)propyl]-N-methyl-carbamate (Intermediate CBS)

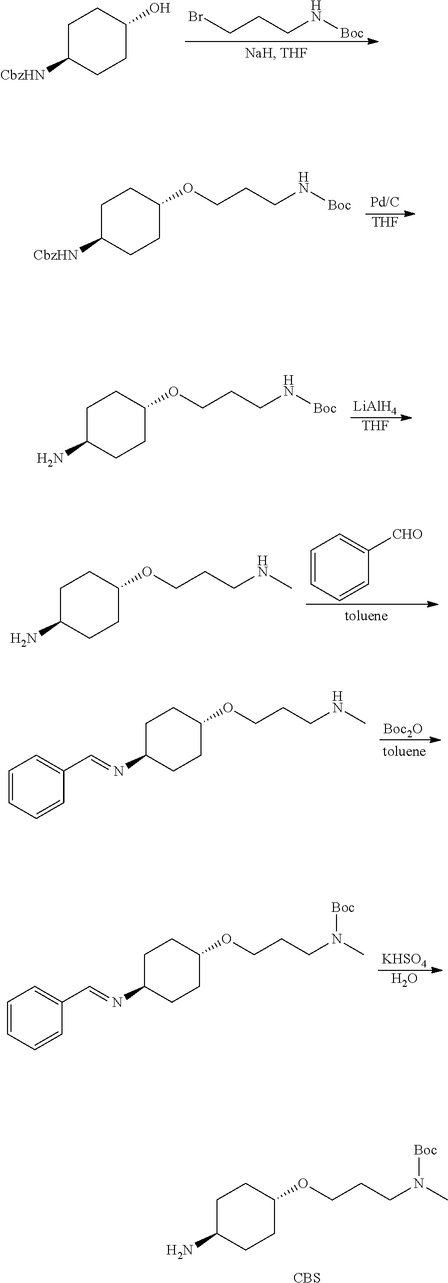

### Step 1—Tert-butyl N-[3-[4-(benzyloxycarbonylamino)cyclohexoxy]propyl]carbamate

To a solution of benzyl N-(4-hydroxycyclohexyl) carbamate (6 g, 24.1 mmol) in DMF (160 mL) was added NaH (1.06 g, 26.4 mmol, 60% dispersion in mineral oil), then the mixture was stirred at 25° C. for 0.5 hour. Next, tert-butyl N-(3-bromopropyl) carbamate (8.60 g, 36.1 mmol, CAS #83948-53-2) was added to the solution stirred at 25° C. for 15.5 hours. On completion, the mixture was quenched with H₂O (150 mL), diluted with H₂O (500 mL), then extracted with DCM (3×200 mL), washed with brine (3×200 mL), dried with anhydrous Na₂SO₄, filtered and the filtrate was concentrated in vacuo. The residue was purified by column chromatography (SiO₂, Petroleum ether/Ethyl acetate=100/1 to 2/1, Rf=65, PE:EA=1:1) to give the title compound (1.2 g, 2.95 mmol, 12% yield) as a white solid. LC-MS (ESI⁺) m/z 307.1 (M+H)+

### Step 2—Tert-butyl N-[3-(4-aminocyclohexoxy)propyl]carbamate

To a solution of Pd/C (10 mg, 49.2 umol, 10 wt %) in THF (2 mL) was added tert-butyl N-[3-[4-(benzyloxycarbonylamino)cyclohexoxy]propyl]carbamate (100 mg, 245 umol). The mixture was stirred at 25° C. for 1 hour under H₂. On completion, the mixture was filtered and the filtrate was concentrated in vacuo to give the title compound (49 mg, 73% yield) as a colorless oily liquid. ¹H NMR (400 MHz, DMSO-d₆). ¹H NMR (400 MHz, CDCl₃) δ 4.89 (s, 1H), 3.52 (t, J=6.0 Hz, 2H), 3.28-3.17 (m, 3H), 2.80-2.69 (m, 1H), 2.09-1.98 (m, 3H), 1.78-1.64 (m, 3H), 1.45 (s, 9H), 1.37-1.11 (m, 6H).

### Step 3—4-[3-(Methylamino)propoxy]cyclohexanamine

A mixture of tert-butyl N-[3-(4-aminocyclohexoxy)propyl]carbamate (760 mg, 2.79 mmol) in THF (13 mL) was degassed and purged with N₂ three times, and then LiAlH₄ (529 mg, 13.9 mmol) was added at 0° C. Then the mixture was stirred at 70° C. for 3 hours under N₂ atmosphere. On completion, the mixture was quenched with H₂O (530 uL) and NaOH (530 uL, 1 mol/L) at 0° C., dried with anhydrous Na₂SO₄ then filtered and washed with EA (50 mL). The filtrate was concentrated in vacuo to give the title compound (500 mg, 96% yield) as a colorless oily liquid. ¹H NMR (400 MHz, DMSO-d6) δ 3.45-3.34 (m, 3H), 3.16-3.08 (m, 1H), 2.48-2.43 (m, 2H), 2.27-2.20 (m, 3H), 1.94-1.83 (m, 1H), 1.84-1.82 (m, 1H), 1.79-1.65 (m, 3H), 1.60-1.52 (m, 2H), 1.36 (d, J=4.0 Hz, 1H), 1.15-0.92 (m, 5H).

### Step 4—3-[4-[(E)-benzylideneamino]cyclohexoxy]-N-methyl-propan-1-amine

A mixture of 4-[3-(methylamino)propoxy]cyclohexanamine (250 mg, 1.34 mmol), benzaldehyde (149 mg, 1.41 mmol, 142 uL, CAS #100-52-7) in toluene (4 mL) was stirred at 120° C. for 16 hours. The mixture was concentrated in vacuo to give the title compound (320 mg, 86% yield) as a brown oily liquid. ¹H NMR (400 MHz, DMSO-d₆) δ 8.38 (s, 1H), 7.89-7.78 (m, 1H), 7.75-7.64 (m, 1H), 7.48-7.37 (m, 2H), 7.32-7.30 (m, 1H), 4.49 (s, 1H), 3.70-3.49 (m, 3H), 2.88-2.59 (m, 3H), 2.10-1.93 (m, 2H), 1.71-1.50 (m, 4H), 1.41-1.18 (m, 4H), 0.92-0.64 (m, 1H), 0.02-0.01 (m, 2H); LC-MS (ESI⁺) m/z 375.0 (M+H)⁺.

### Step 5—Tert-butyl N-[3-[4-[(E)-benzylideneamino]cyclohexoxy]propyl]-N-methyl-carbamate

To a solution of 3-[4-[(E)-benzylideneamino]cyclohexoxy]-N-methyl-propan-1-amine (320 mg, 1.17 mmol) in toluene (8 mL) was added tert-butoxycarbonyl tert-butyl carbonate (254 mg, 1.17 mmol, 267 uL, CAS #24424-99-5). The mixture was then stirred at 25° C. for 3 hours. On completion, the mixture was concentrated in vacuo to give the title compound (400 mg, 910% yield) as a brown oily liquid. LC-MS (ESI⁺) m/z 375.1 (M+H)⁺.

### Step 6—Tert-butyl N-[3-(4-aminocyclohexoxy)propyl]-N-methyl-carbamate

A mixture of tert-butyl N-[3-[4-[(E)-benzylideneamino]cyclohexoxy]propyl]-N-methyl-carbamate (400 mg, 1.07 mmol) and KHSO₄ (290 mg, 2.14 mmol) in H₂O (10 mL) was stirred at 0° C. for 3 hours. On completion, the mixture was diluted with H₂O (10 mL), extracted with MTBE (3×20 mL), and water phase was extracted with DCM (3×20 mL). The combined organic layer was washed with brine (3×20 mL), dried with anhydrous Na₂SO₄, filtered and the filtrate was concentrated in vacuo to give the title compound (220 mg, 71% yield) as a colorless oily liquid. ¹HNMR (400 MHz, DMSO-d₆) δ 3.38-3.35 (m, 2H), 3.22-3.14 (m, 2H), 2.78-2.71 (m, 3H), 2.67 (s, 1H), 2.34 (d, J=12.4 Hz, 1H), 2.07 (s, 1H), 1.89 (d, J=10.0 Hz, 2H), 1.76-1.56 (m, 6H), 1.43-1.35 (m, 9H), 1.16-0.95 (m, 4H) LC-MS (ESI⁺) m/z 287.1 (M+H)+

### 2-(2,6-Dioxo-3-piperidyl)-4-[[4-[3-(methylamino)propoxy]cyclohexyl]amino]isoindoline-1,3-dione (Intermediate CBT)

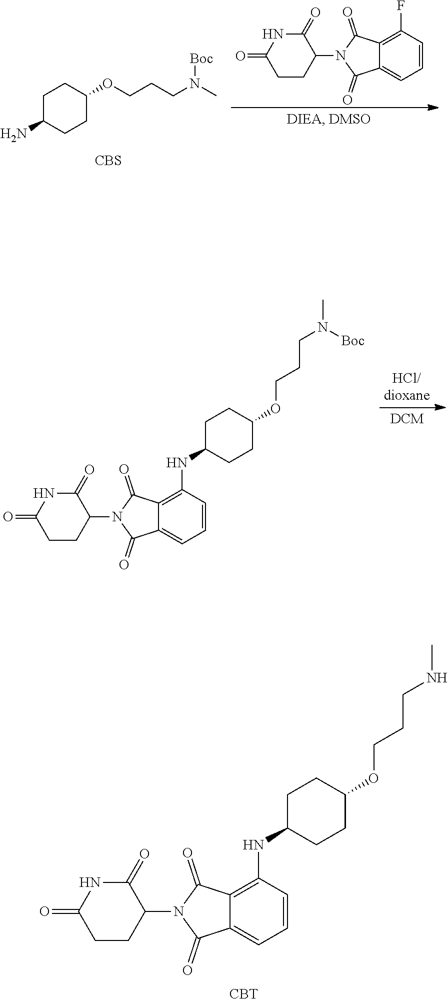

### Step 1—Tert-butyl-N-[3-[4-[[2-(2,6-dioxo-3-piperidyl)-1,3-dioxo-isoindolin-4-yl]amino]cyclohexoxy]propyl]-N-methyl-carbamate

A mixture of tert-butyl N-[3-(4-aminocyclohexoxy)propyl]-N-methyl-carbamate (220 mg, 768 umol, Intermediate CBS), 2-(2,6-dioxo-3-piperidyl)-4-fluoro-isoindoline-1,3-dione (212 mg, 768 umol, CAS #835616-60-9), and DIEA (198 mg, 1.54 mmol, 267 uL) in DMSO (7 mL) was stirred at 130° C. for 2 hours. The mixture was diluted with H₂O (10 mL), and extracted with EA(3×30 mL). The combined organic layer was washed with brine (3×30 mL), dried with anhydrous Na₂SO₄, then filtered and the filtrate was concentrated in vacuo. The mixture was purified by pre-HPLC(column: Phenomenex Luna C18 200*40 mm*10 um; mobile phase: [water(FA)-ACN]; B %: 48%-78%, 8 min) to give the title compound (60 mg, 14% yield) as a yellow solid. ¹H NMR (400 MHz, DMSO-d₆) δ 11.09 (s, 1H), 7.71-7.53 (m, 1H), 7.17 (d, J=8.8 Hz, 1H), 7.04 (d, J=6.8 Hz, 1H), 6.19 (d, J=8.4 Hz, 1H), 5.04 (dd, J=5.2, 12.8 Hz, 1H), 3.21 (t, J=7.2 Hz, 4H), 2.77 (s, 4H), 2.67 (d, J=2.0 Hz, 3H), 2.35-2.31 (m, 1H), 2.09-1.92 (m, 6H), 1.70-1.61 (m, 2H), 1.39 (s, 9H), 1.37-1.29 (m, 4H), LC-MS (ESI⁺) m/z 287.1 (M+H)⁺.

### Step 2—2-(2,6-Dioxo-3-piperidyl)-4-[[4-[3-(methylamino)propoxy]cyclohexyl]amino]isoindoline-1,3-dione

To a solution of tert-butyl N-[3-[4-[[2-(2,6-dioxo-3-piperidyl)-1,3-dioxo-isoindolin-4-yl]amino]cyclohexoxy]propyl]-N-methyl-carbamate (60 mg, 111 umol) in DCM (2 mL) was added HCl/dioxane (4 M, 1 mL). The mixture was then stirred at 25° C. for 1 hour. The mixture was concentrated in vacuo to give the title compound (48 mg, 98% yield) as a yellow solid. LC-MS (ESI⁺) m/z 443.3 (M+H)⁺.

### 4-(2-aminoethylamino)-2-(2,6-dioxo-3-piperidyl)isoindoline-1,3-dione (Intermediate CBU)

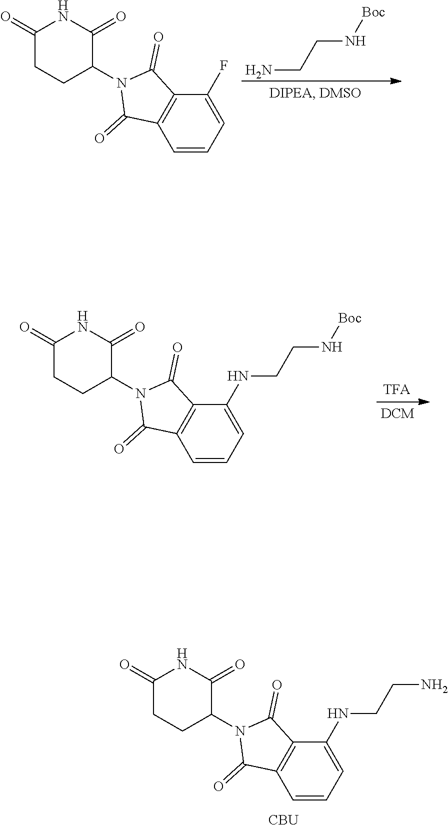

### Step 1—Tert-butyl N-[2-[[2-(2,6-dioxo-3-piperidyl)-1,3-dioxo-isoindolin-4-yl]amino]ethyl]carbamate

To a solution of 2-(2,6-dioxo-3-piperidyl)-4-fluoro-isoindoline-1,3-dione (300 mg, 1.09 mmol) and tert-butyl N-(2-aminoethyl)carbamate (208 mg, 1.30 mmol, 204 uL) in DMSO (3.0 mL) was added DIEA (421 mg, 3.26 mmol, 567 uL). The mixture was then stirred at 130° C. for 1 hour. On completion, the mixture was added H₂O and filtered. The solid was dried to give the title compound (470 mg, 85% yield) as a green solid. ¹H NMR (400 MHz, DMSO-d₆) δ 11.08 (s, 1H), 7.61-7.54 (m, 1H), 7.14 (d, J=8.4 Hz, 1H), 7.03 (d, J=7.2 Hz, 1H), 6.71 (t, J=6.0 Hz, 1H), 5.05 (dd, J=5.6, 12.8 Hz, 1H), 3.36 (d, J=6.4 Hz, 2H), 3.16-3.07 (m, 3H), 2.95-2.83 (m, 1H), 2.60 (d, J=2.4 Hz, 2H), 2.05-1.96 (m, 1H), 1.36 (s, 9H). LC-MS (ESI⁺) m/z 417.0 (M+H)⁺.

### Step 2—4-(2-Aminoethylamino)-2-(2,6-dioxo-3-piperidyl)isoindoline-1,3-dione

To a solution of tert-butyl N-[2-[[2-(2,6-dioxo-3-piperidyl)-1,3-dioxo-isoindolin-4-yl]amino]ethyl]carbamate (150 mg, 360 umol) in DCM (1.00 mL) was added TFA (1.54 g, 13.5 mmol, 1 mL). The mixture was then stirred at 25° C. for 0.5 hours. On completion, the mixture was concentrated to give the title compound (113 mg, 99% yield) as a brown oil. LC-MS (ESI+) m/z 317.0 (M+H)⁺.

### Benzyl N-(2-aminospiro[3.5]nonan-7-yl)-N-methyl-carbamate (Intermediate AXA)

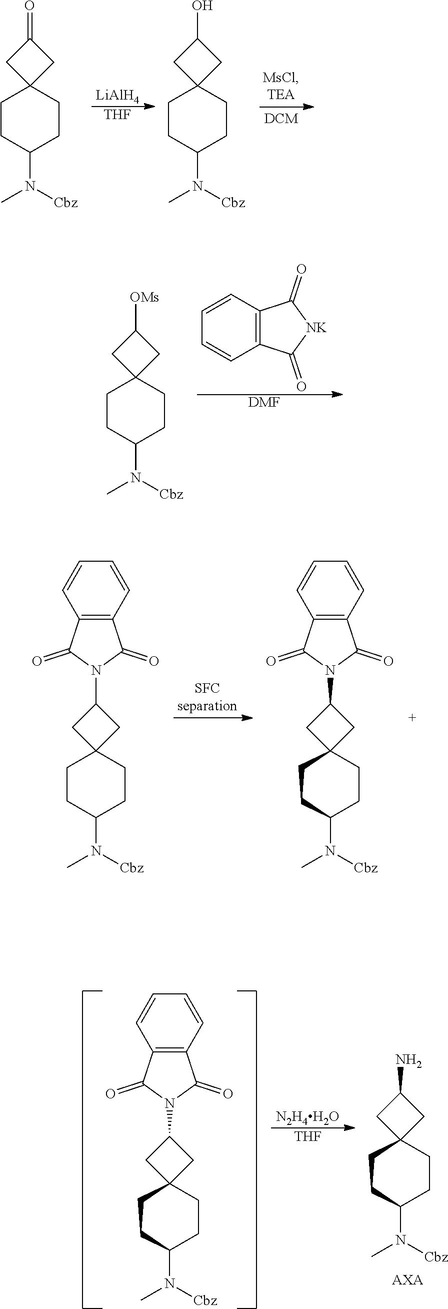

### Step 1—Benzyl N-(2-hydroxyspiro[3.5]nonan-7-yl)-N-methyl-carbamate

To a solution of benzyl N-methyl-N-(2-oxospiro[3.5]nonan-7-yl)carbamate (1.00 g, 3.32 mmol, synthesized via Steps 1-5 of Intermediate ANJ) in THF (15 mL) was added LiAlH₄ (151 mg, 3.98 mmol) at 0° C. The mixture was stirred at 0° C. for 0.1 hr. On completion, the reaction mixture was quenched with water (0.5 mL) and NaOH (0.5 mL, 15%) at 0° C., then the mixture was stirred at 0° C. for 0.5 hr. Then the mixture was filtered and the filtrate was dried over Na₂SO₄, filtered and concentrated in vacuo to give the title compound (1.00 g, 95% yield) as colorless oil. ¹H NMR (400 MHz, CDCl₃) δ 7.44-7.29 (m, 5H), 5.13 (s, 2H), 4.34-4.23 (m, 1H), 3.95 (s, 1H), 3.75 (t, J=6.4 Hz, 1H), 2.79 (s, 3H), 2.38-2.27 (m, 1H), 2.17-2.11 (m, 1H), 1.87-1.84 (m, 1H), 1.77-1.69 (m, 2H), 1.61-1.42 (m, 7H).

### Step 2—[7-[Benzyloxycarbonyl(methyl)amino]spiro[3.5]nonan-2-yl]methanesulfonate

To a solution of benzyl N-(2-hydroxyspiro[3.5]nonan-7-yl)-N-methyl-carbamate (1.00 g, 3.30 mmol) and TEA (1.00 g, 9.89 mmol) in DCM (20 mL) was added MsCl (566 mg, 4.94 mmol) at 0° C. The mixture was stirred at 30° C. for 0.5 hr. On completion, the mixture was quenched with water (20 mL), then washed with water (3×20 mL). The organic layer were dried over Na₂SO₄, filtered and concentrated in vacuo to give the title compound (1.20 g, 95% yield) as colorless oil. ¹H NMR (400 MHz, CDCl₃) δ 7.48-7.28 (m, 5H), 5.13 (s, 2H), 5.04-4.92 (m, 1H), 4.04-3.75 (m, 1H), 3.01-2.96 (s, 3H), 2.78 (s, 3H), 2.50-2.40 (m, 1H), 2.30-2.24 (m, 1H), 2.13-2.05 (m, 1H), 2.00 (dd, J=7.2, 12.0 Hz, 1H), 1.77-1.68 (m, 2H), 1.61-1.52 (m, 3H), 1.52-1.41 (m, 3H).

### Step 3—Benzyl N-[2-(1,3-dioxoisoindolin-2-yl)spiro[3.5]nonan-7-yl]-N-methyl-carbamate

To a solution of [7-[benzyloxycarbonyl(methyl)amino]spiro[3.5]nonan-2-yl]methanesulfonate (1.10 g, 2.88 mmol) in DMF (15 mL) was added (1,3-dioxoisoindolin-2-yl)potassium (801 mg, 4.33 mmol). The mixture was stirred at 100° C. for 16 hrs. On completion, the reaction mixture diluted with water (50 mL) and extracted with EA (3×20 mL). The combined organic layers were dried over Na₂SO₄, filtered and concentrated in vacuo to give a residue. On completion, the reaction mixture was purified by prep-HPLC (column: Phenomenex luna C18 150*40 mm*15 um; mobile phase: [water (0.225% FA)-ACN]) to give the title compound (0.35 g, 28% yield) as light yellow solid. ¹H NMR (400 MHz, CDCl₃) δ 7.88-7.76 (m, 2H), 7.75-7.66 (m, 2H), 7.44-7.28 (m, 5H), 5.15 (s, 2H), 4.78-4.69 (m, 1H), 4.11-3.72 (m, 1H), 2.79 (s, 3H), 2.73-2.65 (m, 1H), 2.59 (t, J=10.0 Hz, 1H), 2.31-2.17 (m, 1H), 2.12-1.99 (m, 2H), 1.98-1.87 (m, 1H), 1.64-1.35 (m, 6H); LC-MS (ESI⁺) m/z 433.4 (M+H)⁺.

### Step 4—Benzyl ((2S,4s,7S)-2-(1,3-dioxoisoindolin-2-yl)spiro[3.5]nonan-7-yl)(methyl)carbamate and benzyl ((2R,4r,7R)-2-(1,3-dioxoisoindolin-2-yl)spiro[3.5]nonan-7-yl)(methyl)carbamate

Benzyl N-[2-(1,3-dioxoisoindolin-2-yl)spiro[3.5]nonan-7-yl]-N-methyl-carbamate (0.30 g, 693 umol) was purified by SFC (column: DAICEL CHIRALPAK AD (250 mm*30 mm, 10 um); mobile phase: [0.1% NH₃·H₂O EtOH]; B %: 60%-60%, 4.4 min) to give to give two isomers. The first fraction was benzyl ((2S,4s,7S)-2-(1,3-dioxoisoindolin-2-yl)spiro[3.5]nonan-7-yl)(methyl)carbamate (120 mg, 38% yield, tR=1.50) isolated as yellow solid. ¹H NMR (400 MHz, CDCl₃) δ 7.75-7.73 (m, 2H), 7.67-7.58 (m, 2H), 7.35-7.19 (m, 5H), 5.07 (s, 2H), 4.70-4.61 (m, 1H), 4.04-3.71 (m, 1H), 2.72 (s, 3H), 2.62 (dd, J=9.6, 10.8 Hz, 1H), 2.51 (t, J=10.0 Hz, 1H), 2.24-2.11 (m, 1H), 2.03-1.92 (m, 2H), 1.88-1.84 (m, 1H), 1.72-1.31 (m, 6H); LC-MS (ESI⁺) m/z 433.0 (M+H)⁺. The second fraction was benzyl ((2R,4r,7R)-2-(1,3-dioxoisoindolin-2-yl) spiro[3.5]nonan-7-yl)(methyl)carbamate (120 mg, 38% yield, tR=1.89) isolated as yellow solid. ¹HNMR (400 MHz, CDCl₃) δ 7.79-7.70 (m, 2H), 7.68-7.59 (m, 2H), 7.36-7.20 (m, 5H), 5.07 (s, 2H), 4.70-4.61 (m, 1H), 4.02-3.73 (m, 1H), 2.72 (s, 3H), 2.62 (dd, J=9.6, 10.8 Hz, 1H), 2.51 (t, J=10.0 Hz, 1H), 2.22-2.11 (m, 1H), 2.05-1.92 (m, 2H), 1.88-1.84 (m, 1H), 1.67-1.32 (m, 6H); LC-MS (ESI⁺) m/z 433.0 (M+H)⁺.

### Step 5—Benzyl N-(2-aminospiro[3.5]nonan-7-yl)-N-methyl-carbamate

To a solution of benzyl N-[2-(1,3-dioxoisoindolin-2-yl)spiro[3.5]nonan-7-yl]-N-methyl-carbamate (0.11 g, 254 umol) in EtOH (3 mL) was added N₂H4H₂O (129 mg, 2.54 mmol, 98% purity). The mixture was stirred at 80° C. for 6 hrs. On completion, the reaction mixture was filtered to give the filtrate and concentrated in vacuo to give a residue. The residue was diluted with DCM (10 mL), filtered to give the filtrate and concentrated in vacuo to give the title compound (75.0 mg, 92% yield) as light yellow solid. ¹H NMR (400 MHz, CDCl₃) δ 7.45-7.28 (m, 5H), 5.13 (s, 2H), 4.06-3.74 (m, 1H), 3.44-3.36 (m, 1H), 2.78 (s, 3H), 2.34-2.23 (m, 1H), 2.11-2.05 (m, 1H), 1.76-1.68 (m, 1H), 1.63-1.36 (m, 9H); LC-MS (ESI⁺) m/z 303.1 (M+H)⁺.

### 2-(2,6-Dioxo-3-piperidyl)-4-[[7-(methylamino)spiro[3.5]nonan-2-yl]amino]isoindoline-1,3-dione (Intermediate AXB)

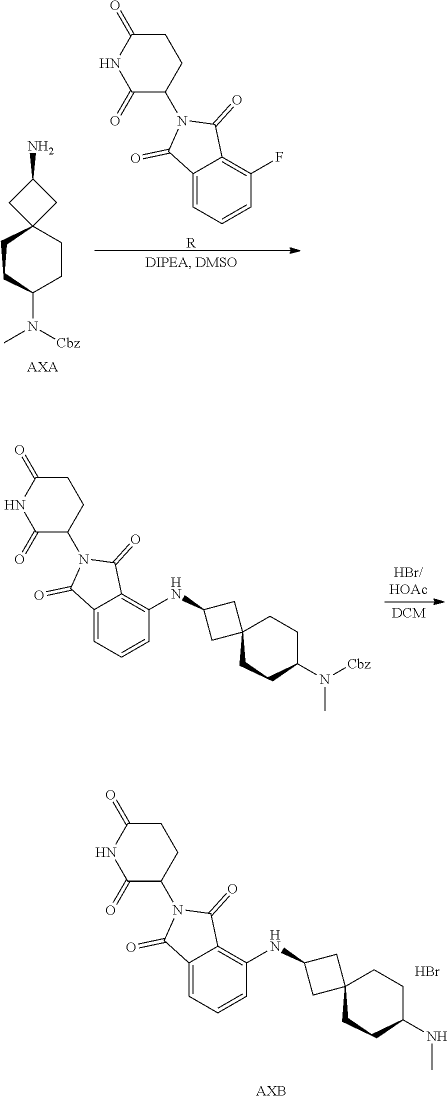

### Step 1—Benzyl N-[2-[[2-(2,6-dioxo-3-piperidyl)-1,3-dioxo-isoindolin-4-yl]amino]spiro[3.5]nonan-7-yl]-N-methyl-carbamate

To a solution of benzyl N-(2-aminospiro[3.5]nonan-7-yl)-N-methyl-carbamate (70.0 mg, 231 umol, Intermediate AXA) and 2-(2,6-dioxo-3-piperidyl)-4-fluoro-isoindoline-1,3-dione (83.1 mg, 300 umol, Intermediate R) in DMSO (2 mL) was added DIPEA (89.7 mg, 694 umol). The mixture was stirred at 130° C. for 3 hrs. On completion, the reaction mixture was diluted with water (8 mL) and extracted with EA (3×10 mL). The combined organic layers were dried over Na₂SO₄, filtered and concentrated in vacuo to give a residue. The residue was purified by reverse phase (FA condition) to give the title compound (65.0 mg, 50% yield) as yellow solid. ¹H NMR (400 MHz, CDCl₃) δ 8.14 (s, 1H), 7.48 (dd, J=7.2, 8.4 Hz, 1H), 7.43-7.28 (m, 5H), 7.11 (d, J=7.2 Hz, 1H), 6.74 (d, J=8.4 Hz, 1H), 6.30 (d, J=5.6 Hz, 1H), 5.14 (s, 2H), 4.98-4.88 (m, 1H), 4.11-3.78 (m, 2H), 2.95-2.68 (m, 6H), 2.46 (t, J=7.6 Hz, 1H), 2.32-2.27 (m, 1H), 2.19-2.10 (m, 1H), 1.90-1.82 (m, 1H), 1.79-1.65 (m, 4H), 1.59-1.39 (m, 5H); LC-MS (ESI⁺) m/z 559.2 (M+H)⁺.

### Step 2—2-(2,6-Dioxo-3-piperidyl)-4-[[7-(methylamino)spiro[3.5]nonan-2-yl]amino]isoindoline-1,3-dione

To a solution of benzyl N-[2-[[2-(2,6-dioxo-3-piperidyl)-1,3-dioxo-isoindolin-4-yl]amino]spiro[3.5]nonan-7-yl]-N-methyl-carbamate (60.0 mg, 107 umol) in DCM (2 mL) was added HBr/HOAc (107 umol, 1 mL, 33% solution). The mixture was stirred at 25° C. for 2 hrs. On completion, the mixture was concentrated under N₂ flow to give the title compound (50.0 mg, 90% yield, HBr salt) as yellow solid. LC-MS (ESI⁺) m/z 425.2 (M+H)⁺.

### N-[2-(4-formylphenyl)-6-(1-hydroxy-1-methyl-ethyl)indazol-5-yl]-6-(trifluoromethyl)pyridine-2-carboxamide (Intermediate CBW)

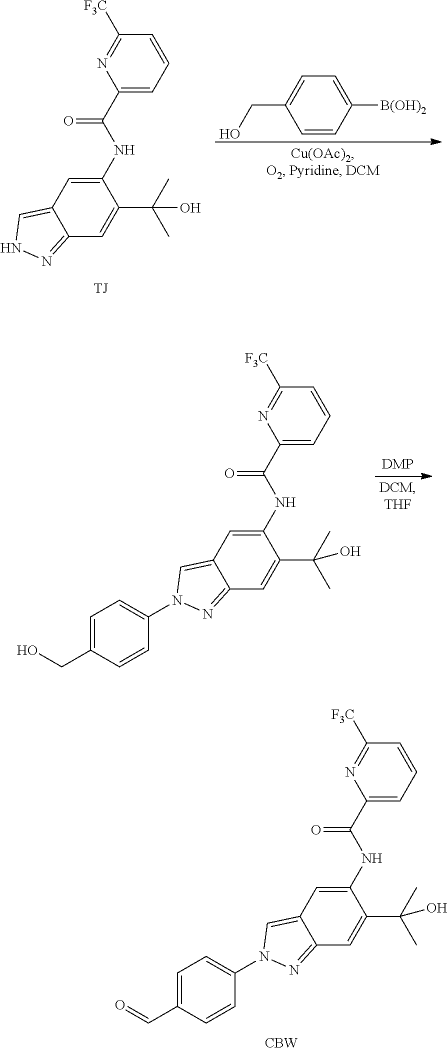

### Step 1—N-[6-(1-hydroxy-1-methyl-ethyl)-2-[4-(hydroxymethyl)phenyl]indazol-5-yl]-6-(trifluoromethyl)pyridine-2-carboxamide

To a solution of N-[6-(1-hydroxy-1-methyl-ethyl)-2H-indazol-5-yl]-6-(rifluoromethyl)pyridine-2-carboxamide (300 mg, 824 umol, Intermediate TJ) and [4-(hydroxymethyl)phenyl]boronic acid (150 mg, 988 umol CAS #59016-93-2) in DCM (10 mL) was added Cu(OAc)₂ (179 mg, 988 umol) and pyridine (390 mg, 4.94 mmol). The mixture was stirred at 25° C. for 72 hours under O₂ atmosphere. On completion, the mixture was filtered and washed with DCM (3×10 mL). The filtrate was adjust pH=5 with HCl (2 mL, 1 mol/L), washed with H₂O (3×10 mL) then washed with brine (3×10 mL), dried with anhydrous Na₂SO₄, filtered and the filtrate was concentrated in vacuo. The residue was purified by column chromatography (SiO₂, Petroleum ether/Ethyl acetate=100/1 to 1/1) to give the title compound (47.0 mg, 99.9 umol, 12% yield) as a yellow solid. ¹H NMR (400 MHz, DMSO-d₆) δ 12.46 (s, 1H), 9.06 (s, 1H), 8.83 (s, 1H), 8.47 (d, J=7.6 Hz, 1H), 8.38 (t, J=7.6 Hz, 1H), 8.17 (d, J=7.2 Hz, 1H), 8.03 (d, J=8.4 Hz, 2H), 7.70 (s, 1H), 7.52 (d, J=8.4 Hz, 2H), 6.05 (s, 1H), 5.32 (t, J=5.6 Hz, 1H), 4.58 (d, J=5.6 Hz, 2H), 1.66 (s, 6H). LC-MS (ESI⁺) m/z 471.3 (M+H)⁺.

### Step 2—N-[2-(4-formylphenyl)-6-(1-hydroxy-1-methyl-ethyl)indazol-5-yl]-6-(trifluoromethyl)pyridine-2-carboxamide

To a solution of N-[6-(1-hydroxy-1-methyl-ethyl)-2-[4-(hydroxymethyl)phenyl]indazol-5-yl]-6-(trifluoromethyl)pyridine-2-carboxamide (57.0 mg, 121 umol) in DCM (1 mL) and THF (1 mL) was added DMP (77.1 mg, 181 umol, 56.2 uL). The mixture was then stirred at 25° C. for 1 hour. On completion, the mixture was diluted with DCM (5 mL), and quenched with Na₂S₂O₃ (10 mL) and NaHCO₃ (10 mL). The combined organic layer was washed with brine (3×10 mL), then dried with anhydrous Na₂SO₄, filtered and the filtrate was concentrated in vacuo. The crude product was purified by pre-HPLC (column: Phenomenex luna C18 150*25 mm*10 um; mobile phase: [water(FA)-ACN]; B %: 47%-77%, 9 min) to give the title compound (10 mg, 18% yield) as a yellow solid. ¹HNMR (400 MHz, DMSO-d₆) δ 12.49 (s, 1H), 10.08 (s, 1H), 9.24 (s, 1H), 8.85 (s, 1H), 8.47 (d, J=7.6 Hz, 1H), 8.38 (s, 1H), 8.35 (s, 1H), 8.33 (s, 1H), 8.18 (d, J=7.6 Hz, 1H), 8.13 (d, J=8.6 Hz, 2H), 6.09 (s, 1H), 2.35-2.31 (m, 1H), 1.66 (s, 6H), LC-MS(ESI⁺) m/z 469.0 (M+H)⁺.

### 3-[5-methoxy-3-methyl-2-oxo-4-(1,2,3,6-tetrahydropyridin-4-yl)benzimidazol-1-yl]piperidine-2,6-dione (Intermediate CBX)

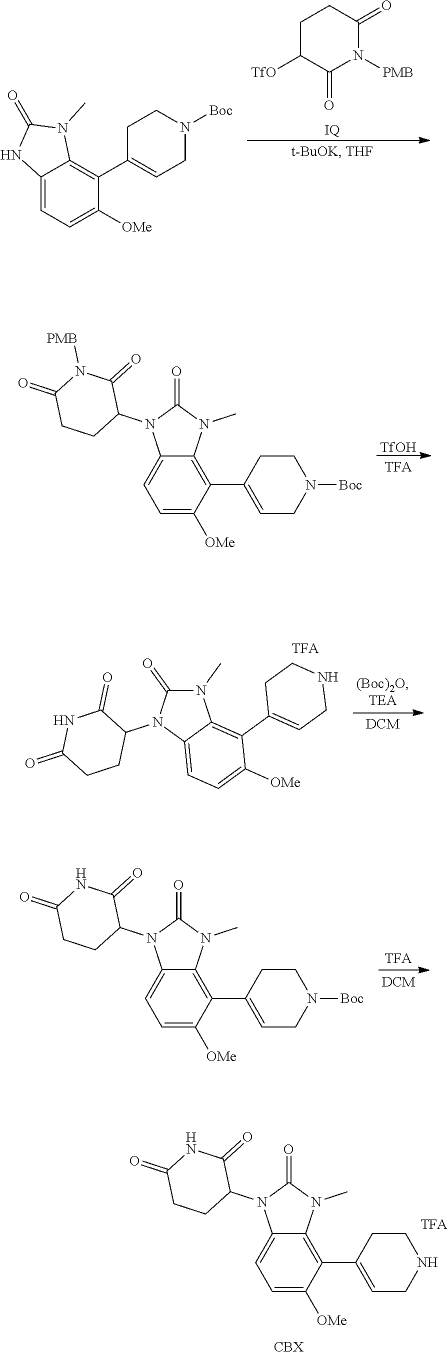

### Step 1—Tert-butyl 4-[5-methoxy-1-[1-[(4-methoxyphenyl)methyl]-2,6-dioxo-3-piperidyl]-3-methyl-2-oxo-benzimidazol-4-yl]-3,6-dihydro-2H-pyridine-1-carboxylate

To a solution of tert-butyl 4-(5-methoxy-3-methyl-2-oxo-1H-benzimidazol-4-yl)-3,6-dihydro-2H-pyridine-1-carboxylate (500 mg, 1.39 mmol, synthesized via Step 1 of Intermediate BUC) in THF (10 mL) was added t-BuOK (234 mg, 2.09 mmol) at 0° C. The mixture was stirred at 0° C. for 0.5 hr. Then, to the above mixture was added [1-[(4-methoxyphenyl)methyl]-2,6-dioxo-3-piperidyl]trifluoromethanesulfonate (795 mg, 2.09 mmol, Intermediate IQ) in THF (4 mL). The mixture was stirred at 0° C. for 3.5 hrs. On completion, the reaction was quenched with NH₄Cl solution (5 mL), and extracted with EA (100×2 mL). The organic layer was washed with water (100 mL×3), dried over Na₂SO₄ and filtered. The filtrate was concentrated in vacuo. The residue was purified by column chromatography (SiO₂, Petroleum ether/Ethyl acetate=3/2 to 1/2) to give the title compound (750 mg, 91% yield) as blue solid. ¹H NMR (400 MHz, DMSO-d₆) δ=7.21 (d, J=8.4 Hz, 2H), 6.86 (d, J=8.4 Hz, 3H), 6.64 (dd, J=3.6, 8.4 Hz, 1H), 5.62 (s, 1H), 5.49 (dd, J=5.2, 13.2 Hz, 1H), 4.91-4.70 (m, 2H), 4.02-3.89 (m, 2H), 3.72 (s, 3H), 3.71 (s, 3H), 3.62-3.46 (m, 2H), 3.26 (s, 3H), 3.10-2.99 (m, 1H), 2.86-2.64 (m, 2H), 2.43 (s, 1H), 2.24-2.13 (m, 1H), 2.08-2.00 (m, 1H), 1.44 (s, 9H).

### Step 2—3-[5-methoxy-3-methyl-2-oxo-4-(1,2,3,6-tetrahydropyridin-4-yl)benzimidazol-1-yl]piperidine-2,6-dione

A mixture of tert-butyl 4-[5-methoxy-1-[1-[(4-methoxyphenyl)methyl]-2,6-dioxo-3-piperidyl]-3-methyl-2-oxo-benzimidazol-4-yl]-3,6-dihydro-2H-pyridine-1-carboxylate (100 mg, 169 umol) and TfOH (340 mg, 2.27 mmol) in TFA (1 mL) was stirred at 70° C. for 1 hr. On completion, the reaction was concentrated in vacuo to give the title compound (82.0 mg, 99% yield, TFA) as brown oil. LC-MS (ESI⁺) m/z 371.1 (M+H)⁺.

### Step 3—Tert-butyl 4-[1-(2,6-dioxo-3-piperidyl)-5-methoxy-3-methyl-2-oxo-benzimidazol-4-yl]-3,6-dihydro-2H-pyridine-1-carboxylate

To a mixture of 3-[5-methoxy-3-methyl-2-oxo-4-(1,2,3,6-tetrahydropyridin-4-yl)benzimidazol-1-yl]piperidine-2,6-dione (82.0 mg, 169 umol, TFA) and TEA (51.3 mg, 507 umol) in DCM (2 mL) was added Boc₂O (55.4 mg, 253 umol). The mixture was then stirred at 25° C. for 16 hrs. On completion, the reaction was diluted with DCM (50 mL). The organic layer was washed with water (50 mL×3), dried over Na₂SO₄ and filtered. The filtrate was concentrated in vacuo. The residue was purified by prep-HPLC (column: Phenomenex luna C18 150*25 mm*10 um; mobile phase: [water (FA)-ACN]; B %: 33%-63%, 10 min) to give the title compound (40.0 mg, 50% yield) as white solid. ¹H NMR (400 MHz, DMSO-d₆) δ 11.09 (s, 1H), 6.98 (dd, J=1.6, 8.4 Hz, 1H), 6.70 (d, J=8.4 Hz, 1H), 5.64 (s, 1H), 5.34 (dd, J=5.2, 12.4 Hz, 1H), 4.08-3.91 (m, 2H), 3.72 (s, 3H), 3.65-3.47 (m, 2H), 3.27 (s, 3H), 2.96-2.83 (m, 1H), 2.76-2.62 (m, 2H), 2.45 (d, J=8.0 Hz, 1H), 2.26-2.13 (m, 1H), 2.05-1.94 (m, 1H), 1.44 (s, 9H).

### Step 4—3-[5-methoxy-3-methyl-2-oxo-4-(1,2,3,6-tetrahydropyridin-4-yl)benzimidazol-1-yl]piperidine-2,6-dione

A mixture of tert-butyl 4-[1-(2,6-dioxo-3-piperidyl)-5-methoxy-3-methyl-2-oxo-benzimidazol-4-yl]-3,6-dihydro-2H-pyridine-1-carboxylate (40.0 mg, 85.0 umol) and TFA (308 mg, 2.70 mmol) in DCM (1 mL) was stirred at 25° C. for 1 hr. On completion, the reaction was concentrated in vacuo to give the title compound (41.0 mg, 99% yield, TFA) as yellow oil. LC-MS (ESI⁺) m/z 371.2 (M+H)⁺.

### 5-[tert-butyl(diphenyl)silyl]oxypentylmethanesulfonate (Intermediate CBY)

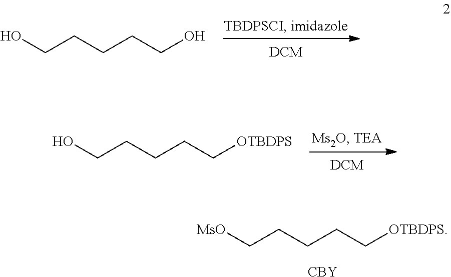

### Step 1—5-[tert-butyl(diphenyl)silyl]oxypentan-1-ol

To a solution of pentane-1, 5-diol (2 g, 19.2 mmol, 2.02 mL, from CAS #111-29-5) in DCM (20 mL) was added imidazole (522 mg, 7.68 mmol) and TBDPSCl (1.06 g, 3.84 mmol, 986 uL) at 0° C. The mixture was then stirred at 25° C. for 3 hours. On completion, the reaction mixture was concentrated under reduced pressure to give the title compound (1.20 g, 91% yield) as colorless oil.

### Step 2—5-[tert-butyl(diphenyl)silyl]oxypentyl methanesulfonate

To a solution of 5-[tert-butyl(diphenyl)silyl]oxypentan-1-ol (300 mg, 875 umol) in DCM (3 mL) was added TEA (177 mg, 1.75 mmol, 243 uL) and methylsulfonyl methanesulfonate (228 mg, 1.31 mmol). The mixture was then stirred at 25° C. for 2 hours. On completion, the reaction mixture was concentrated under reduced pressure to give the title compound (210 mg, 57% yield) as colorless oil. LC-MS (ESI⁺) m/z 421.2 (M+H)⁺.

### N-[6-(1-hydroxy-1-methyl-ethyl)-2-(5-oxopentyl)indazol-5-yl]-6-(trifluoromethyl)pyridine-2-carboxamide (Intermediate CBZ)

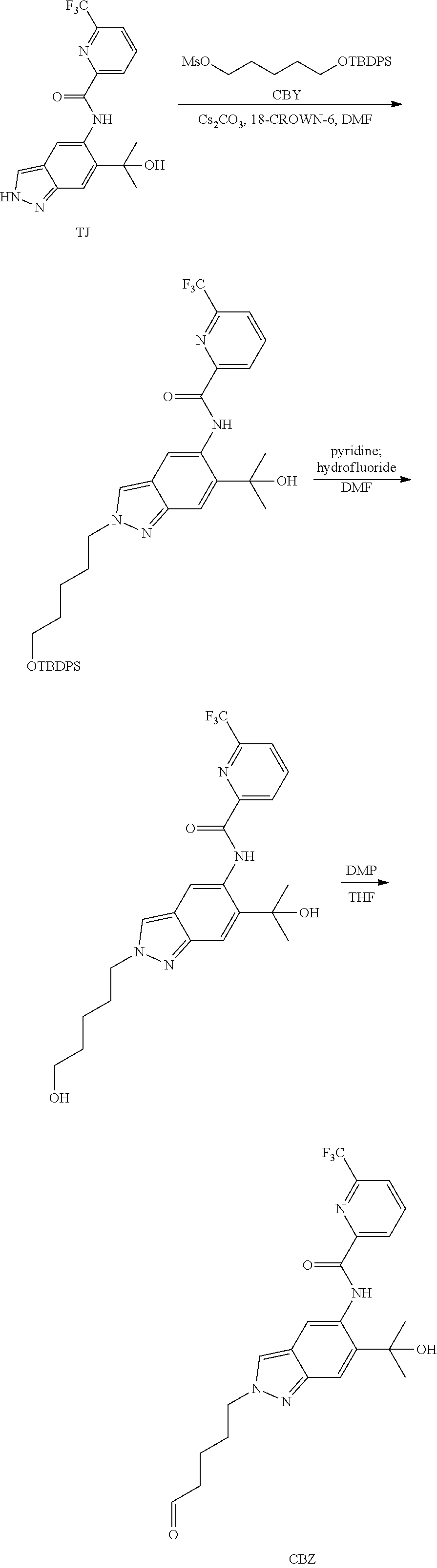

### Step 1—N-[2-[5-[tert-butyl(diphenyl)silyl]oxypentyl]-6-(1-hydroxy-1-methyl-ethyl)indazol-5-yl]-6-(trifluoromethyl)pyridine-2-carboxamide

To a solution of 5-[tert-butyl(diphenyl)silyl]oxypentyl methanesulfonate (200 mg, 475 umol, Intermediate CBY) in DMF (3 mL) was added N-[6-(1-hydroxy-1-methyl-ethyl)-2H-indazol-5-yl]-6-(trifluoromethyl)pyridine-2-carboxamide (86.6 mg, 237 umol, Intermediate TJ), Cs₂CO₃ (232 mg, 713 umol) and 18-CROWN-6 (12.5 mg, 47.5 umol). The mixture was stirred at 80° C. for 17 hours. On completion, the reaction mixture concentrated under reduced pressure to give the title compound (115 mg, 70% yield) as white solid. LC-MS (ESI⁺) m/z 689.3 (M+H)⁺.

### Step 2—N-[6-(1-hydroxy-1-methyl-ethyl)-2-(5-hydroxypentyl)indazol-5-yl]-6-(trifluoromethyl) pyridine-2-carboxamide

To a solution of N-[2-[5-[tert-butyl(diphenyl)silyl]oxypentyl]-6-(1-hydroxy-1-methyl-ethyl)indazol-5-yl]-6-(trifluoromethyl)pyridine-2-carboxamide (300 mg, 435 umol) in DMF (3 mL) was added pyridine; hydrofluoride (43.1 mg, 435 umol, 39.2 uL). The mixture was stirred at 25° C. for 2 hours. On completion, the reaction mixture was concentrated under reduced pressure to give a residue. The residue was purified by prep-HPLC (column: Phenomenex luna C18 150*25 mm*10 um; mobile phase: [water(FA)-ACN]; B %: 33%-63%, 9 min) to give the title compound (37 mg, 19% yield) as colorless oil. LC-MS (ESI⁺) m/z 451.4 (M+H)⁺. ¹H NMR (400 MHz, CDCl₃) δ 12.28 (s, 1H), 8.87 (s, 1H), 8.51 (d, J=8.0 Hz, 1H), 8.11 (t, J=8.0 Hz, 1H), 7.89 (s, 1H), 7.85 (d, J=7.6 Hz, 1H), 7.74 (s, 1H), 4.43 (t, J=7.2 Hz, 2H), 3.65 (t, J=6.4 Hz, 2H), 2.37-2.16 (m, 1H), 2.10-2.02 (m, 2H), 1.81 (s, 7H), 1.64 (d, J=7.2 Hz, 2H), 1.47-1.39 (m, 2H).

### Step 3—N-[6-(1-hydroxy-1-methyl-ethyl)-2-(5-oxopentyl)indazol-5-yl]-6-(trifluoromethyl)pyridine-2-carboxamide

To a solution of N-[6-(1-hydroxy-1-methyl-ethyl)-2-(5-hydroxypentyl)indazol-5-yl]-6-(trifluoromethyl)pyridine-2-carboxamide (30 mg, 66.6 umol) in THF (2 mL) was added DMP (48.0 mg, 113 umol, 35.0 uL) at 0° C. The mixture was then stirred at 25° C. for 32 hours. On completion, the reaction mixture concentrated under reduced pressure to give the title compound (29.0 mg, 97% yield) as white oil. LC-MS (ESI⁺) m/z 449.0 (M+H)⁺.

### 2-Benzyl N-methyl-N-[3-[3-(methylamino)cyclobutoxy]propyl]carbamate (Intermediate CCA)

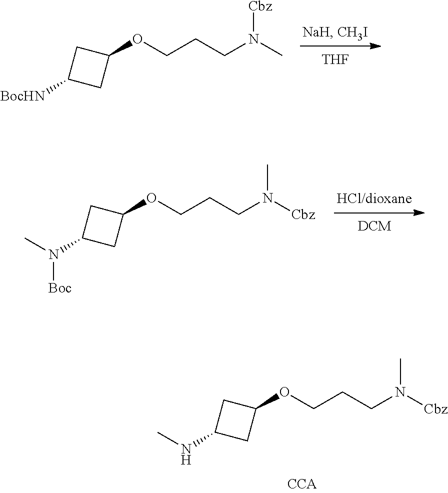

### Step 1—Tert-butyl N-[3-[3-[benzyloxycarbonyl(methyl)amino]propoxy]cyclobutyl]-N-methyl-carbamate

To a solution of benzyl N-[3-[3-(tert-butoxycarbonylamino)cyclobutoxy]propyl]-N-methyl-carbamate (1 g, 2.55 mmol, synthesized via Steps 1-5 of Intermediate AOY) in THF (10 mL) was added NaH (305 mg, 7.65 mmol, 60% dispersion in mineral oil) at 0° C. for 30 minutes. Then CH₃I (1.08 g, 7.65 mmol) was added into the solution and the mixture was stirred at 25° C. for 5.5 hours. On completion, the mixture was quenched with H₂O (10 mL), diluted with H₂O (20 mL), and extracted with EA (30 mL×3). The combined organic layer was washed with brine (30 mL×3), dried with anhydrous Na₂SO₄, filtered and concentrated in vacuo to give the title compound (1.00 g, 96% yield) as a yellow oil. ¹H NMR (400 MHz, CDCl₃) δ 7.40-7.28 (m, 5H), 5.14 (s, 2H), 4.83-4.60 (m, 1H), 4.00-3.86 (m, 1H), 3.42-3.35 (m, 2H), 2.95 (s, 3H), 2.81 (s, 3H), 2.66-2.56 (m, 2H), 2.33-2.19 (m, 4H), 1.88-1.75 (m, 2H), 1.46 (s, 9H).

### Step 2—2-Benzyl N-methyl-N-[3-[3-(methylamino)cyclobutoxy]propyl]carbamate

To a solution of tert-butyl N-[3-[3-[benzyloxycarbonyl(methyl)amino]propoxy]cyclobutyl]-N-methyl-carbamate (400 mg, 983 umol) in DCM (8 mL) was added HCl/dioxane (4 M, 4 mL). The mixture was then stirred at 25° C. for 2 hours. On completion, the mixture was concentrated in vacuo to give the title compound (337 mg, 99% yield, HCl) as a yellow oil. LC-MS (ESI⁺) m/z 307.0 (M+H)⁺.

### 2-(2,6-Dioxo-3-piperidyl)-4-[methyl-[3-[3-(methylamino)propoxy]cyclobutyl]amino]isoindoline-1,3-dione (Intermediate CCB)

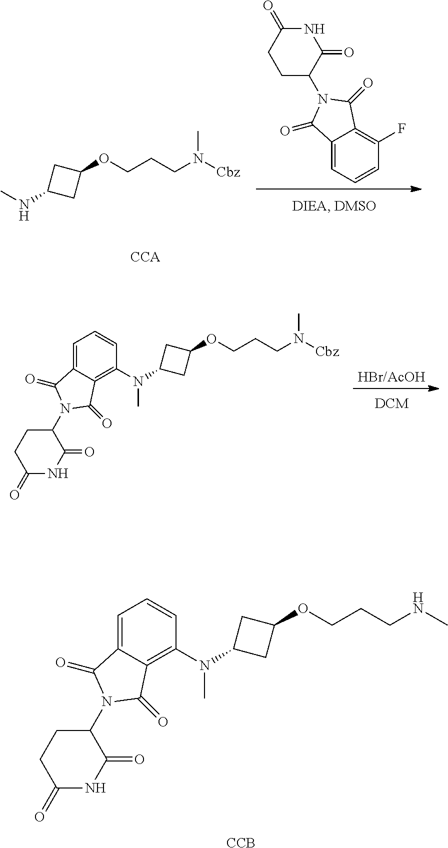 [SMILES: CN[C@H]1C[C@H](OCCCN(C)C(=O)OCc2ccccc2)C1 ; O=C1CCC(N2C(=O)c3cccc(F)c3C2=O)C(=O)N1 ; CN(CCCO[C@H]1C[C@H](N(C)c2cccc3c2C(=O)N(C2CCC(=O)NC2=O)C3=O)C1)C(=O)OCc1ccccc1 ; CNCCCO[C@H]1C[C@H](N(C)c2cccc3c2C(=O)N(C2CCC(=O)NC2=O)C3=O)C1 ; "CCB"]

### Step 1—3-Benzyl N-[3-[3-[[2-(2,6-dioxo-3-piperidyl)-1,3-dioxo-isoindolin-4-yl]-methyl-amino] cyclobutoxy]propyl]-N-methyl-carbamate

To a solution of benzyl N-methyl-N-[3-[3-(methylamino)cyclobutoxy]propyl]carbamate (337 mg, 982 umol, HCl, Intermediate CCA) and 2-(2,6-dioxo-3-piperidyl)-4-fluoro-isoindoline-1,3-dione (226 mg, 819 umol, CAS #835616-60-9) in DMSO (5 mL) was added DIEA (529 mg, 4.10 mmol). The mixture was then stirred at 130° C. for 5 hours. On completion, the mixture was diluted with H₂O (20 mL), and extracted with EA (20 mL×3). The combined organic layer was washed with brine (50 mL×3), dried with anhydrous Na₂SO₄, filtered and concentrated in vacuo. The residue was purified by prep-HPLC (column: Phenomenex Luna C 18 200*40 mm*10 um; mobile phase: [water (FA)-ACN]; B %: 43%-73%, 10 minutes) to give the title compound (206 mg, 44% yield) as a yellow solid. ¹H NMR (400 MHz, DMSO-d₆) δ 11.07 (s, 1H), 7.62 (t, J=7.2 Hz, 1H), 7.40-7.26 (m, 6H), 7.16-7.05 (m, 1H), 5.13-5.07 (m, 1H), 5.05 (s, 2H), 4.32-4.15 (m, 1H), 3.98-3.85 (m, 1H), 3.29-3.22 (m, 3H), 2.93 (s, 3H), 2.89-2.82 (m, 4H), 2.71-2.58 (m, 4H), 2.30-2.20 (m, 4H), 1.76-1.68 (m, 2H). LC-MS (ESI⁺) m/z 563.4 (M+H)⁺.

### Step 2—2-(2,6-Dioxo-3-piperidyl)-4-[methyl-[3-[3-(methylamino)propoxy]cyclobutyl]amino]isoindoline-1,3-dione

To a solution of benzyl N-[3-[3-[[2-(2,6-dioxo-3-piperidyl)-1,3-dioxo-isoindolin-4-yl]-methyl-amino]cyclobutoxy]propyl]-N-methyl-carbamate (100 mg, 177 umol) in DCM (5 mL) was added hydrogen bromide (2.88 g, 10.6 mmol, 30% solution). The mixture was then stirred at 25° C. for 1 hour. On completion, the mixture was dried with N₂, then washed with DCM (4 mL×3) and concentrated in vacuo to give the title compound (90.0 mg, 99% yield, HBr) as a yellow solid. LC-MS (ESI⁺) m/z 429.0 (M+H)⁺.

### 2-(2,6-Dioxo-3-piperidyl)-5-[[3-[3-(methylamino)propoxy]cyclobutyl]amino]isoindoline-1,3-dione (Intermediate CCC)

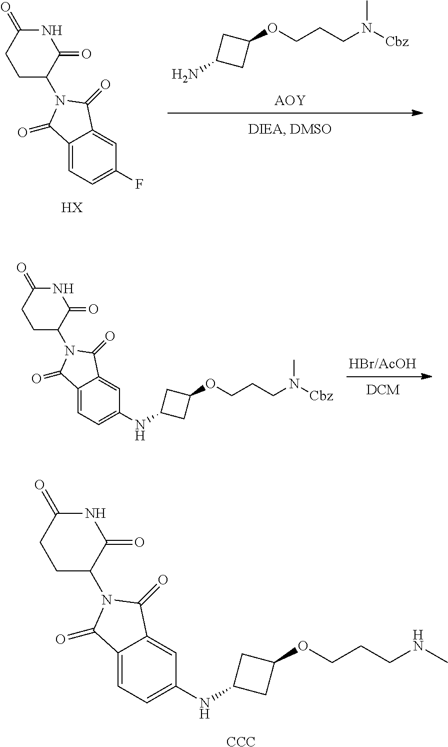

### Step 1—Benzyl N-[3-[3-[[2-(2,6-dioxo-3-piperidyl)-1,3-dioxo-isoindolin-5-yl]amino]cyclobutoxy]propyl]-N-methyl-carbamate

To a solution of benzyl N-[3-(3-aminocyclobutoxy)propyl]-N-methyl-carbamate (190 mg, 577 umol, HCl, Intermediate AOY) and 2-(2,6-dioxo-3-piperidyl)-5-fluoro-isoindoline-1,3-dione (133 mg, 481 umol, Intermediate HX) in DMSO (2 mL) was added DIEA (311 mg, 2.41 mmol, 419 uL). The mixture was then stirred at 130° C. for 3 hours. On completion, the mixture was diluted with H₂O (3 mL) and extracted with EA (3×5 mL). The combined organic layer was washed with brine (3×5 mL), dried with anhydrous Na₂SO₄, then filtered and the filtrate was concentrated in vacuo. The mixture was purified by pre-HPLC (column: Phenomenex Luna C18 200*40 mm*10 um; mobile phase: [water(FA)-ACN]; B %: 38%-68%, 10 min) to give the title compound (35 mg, 13% yield) as a brown solid. LC-MS(ESI⁺) m/z 549.3 (M+H)⁺.

### Step 2—2-(2,6-Dioxo-3-piperidyl)-5-[[3-[3-(methylamino)propoxy]cyclobutyl]amino]isoindoline-1,3-dione

To a solution of benzyl N-[3-[3-[[2-(2,6-dioxo-3-piperidyl)-1,3-dioxo-isoindolin-5-yl]amino]cyclobutoxy]propyl]-N-methyl-carbamate (75.0 mg, 136 umol) in DCM (3 mL) was added HBr/AcOH (2.21 g, 8.20 mmol, 1.48 mL, 30% solution). The mixture was then stirred at 25° C. for 1 hour. On completion the mixture was dried with N₂, then washed with DCM (3×4 mL) and concentrated in vacuo. The mixture was without purification to give the title compound (35 mg, 63.8 umol, 13% yield) as a brown solid. LC-MS (ESI⁺) m/z 415.2 (M+H)⁺.

### Benzyl N-[2-(3-aminocyclobutoxy)ethyl]-N-methyl-carbamate (Intermediate CCD)

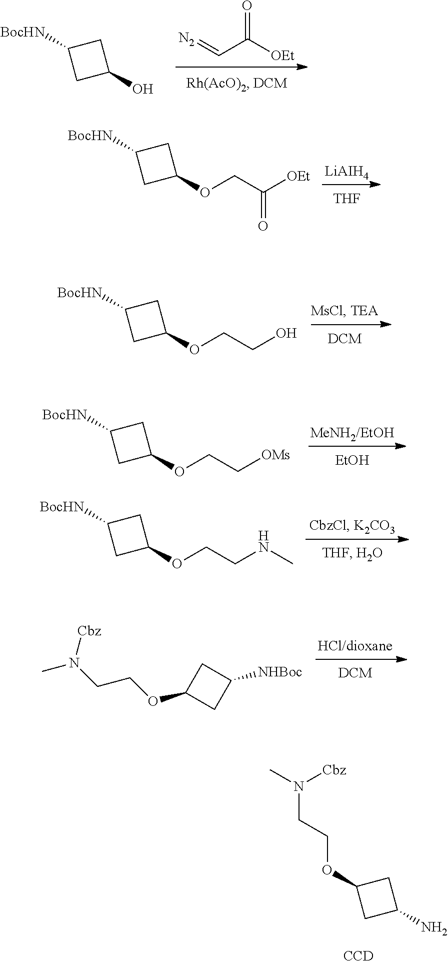

### Step 1—Ethyl 2-[3-(tert-butoxycarbonylamino)cyclobutoxy]acetate

To a solution of tert-butyl N-(3-hydroxycyclobutyl)carbamate (2.5 g, 13.3 mmol) in DCM (80 mL) was added Rh(AcO)₂ (295 mg, 667 umol, CAS #15956-28-2) and ethyl 2-diazoacetate (4.57 g, 40 mmol, CAS #623-73-4). The mixture was then stirred at 25° C. for 16 hours. On completion, the mixture was washed with H₂O (3×100 mL), then washed with brine, and the organic layer was dried with anhydrous Na₂SO₄, filtered and the filtrate was concentrated in vacuo. The residue was purified by column chouromatography (SiO₂, Petroleum ether/Ethyl acetate=100/1 to 5/1) (Rf=0.51, PE:EA=2:1) to give the title compound (0.93 g, 3.40 mmol, 25% yield) as a white solid. ¹H NMR (400 MHz, DMSO-d₆) δ 7.15 (d, J=6.8 Hz, 1H), 4.10 (q, J=7.2 Hz, 3H), 4.01-3.91 (m, 3H), 2.24-2.13 (m, 2H), 2.11-2.01 (m, 2H), 1.36 (s, 9H), 1.19 (t, J=7.2 Hz, 3H).

### Step 2—Tert-butyl N-[3-(2-hydroxyethoxy)cyclobutyl]carbamate

To a solution of ethyl 2-[3-(tert-butoxycarbonylamino)cyclobutoxy]acetate (1.86 g, 6.81 mmol) in THF (20 mL) was added LiAlH₄ (387 mg, 10.2 mmol). The mixture was stirred at 0° C. for 2 hours under N₂, The mixture was quenched with H₂O (0.4 mL) and NaOH (0.4 mL, 10% solution), dried with anhydrous Na₂SO₄ then filtered and the filtrate was concentrated in vacuo to give the title compound (1.28 g, 81% yield) as a white solid. ¹H NMR (400 MHz, DMSO-d₆) δ 7.15 (d, J=6.8 Hz, 1H), 4.57 (t, J=5.2 Hz, 1H), 4.04-3.90 (m, 2H), 3.46 (q, J=5.2 Hz, 2H), 3.29-3.24 (m, 2H), 2.14-2.02 (m, 4H), 1.37 (s, 9H).

### Step 3—2-[3-(Tert-butoxycarbonylamino)cyclobutoxy]ethyl methanesulfonate

To a solution of tert-butyl N-[3-(2-hydroxyethoxy)cyclobutyl]carbamate (200 mg, 864 umol) in DCM (8 mL) was added TEA (262 mg, 2.59 mmol, 361 uL) and methylsulfonyl methanesulfonate (301.26 mg, 1.73 mmol, CAS #7143-01-3). The mixture was stirred at 25° C. for 2 hours. On completion, the mixture was washed with H₂O (3×10 mL), then washed with brine (3×10 mL), dried with anhydrous Na₂SO₄, filtered and the filtrate was concentrated in vacuo to give the title compound (236 mg, 88% yield) as a white solid. ¹H NMR (400 MHz, DMSO-d₆) δ 7.17 (d, J=6.8 Hz, 1H), 4.30-4.27 (m, 2H), 4.10-4.02 (m, 1H), 4.00-3.93 (m, 1H), 3.54-3.49 (m, 2H), 3.18 (s, 3H), 2.18 (d, J=3.6 Hz, 1H), 2.13 (td, J=8.0, 4.0 Hz, 2H), 2.09 (d, J=6.0 Hz, 1H), 1.37 (s, 9H).

### Step 4—Tert-butyl N-[3-[2-(methylamino)ethoxy]cyclobutyl]carbamate

To a solution of 2-[3-(tert-butoxycarbonylamino)cyclobutoxy]ethyl methanesulfonate (236 mg, 762 umol) in EtOH (5 mL) was added MeNH₂ (1.61 g, 15.5 mmol, 2 mL, 30% solution). The mixture was stirred at 80° C. for 16 hrs. On completion, the mixture was concentrated in vacuo to give the title compound (186 mg, 99% yield) as a brown liquid. ¹H NMR (400 MHz, CDCl₃) δ 4.70 (s, 1H), 4.43-4.32 (m, 2H), 4.14 (dt, J=7.2, 3.2 Hz, 2H), 3.63-3.54 (m, 2H), 3.07 (s, 3H), 2.42-2.33 (m, 2H), 2.15 (td, J=12.4, 6.4 Hz, 2H), 1.65-1.57 (m, 1H), 1.44 (s, 9H).

### Step 5-Benzyl N-[2-[3-(tert-butoxycarbonylamino)cyclobutoxy]ethyl]-N-methyl-carbamate

To a solution of tert-butyl N-[3-[2-(methylamino)ethoxy]cyclobutyl]carbamate (186 mg, 761 umol) in THF (10 mL) and H₂O (3 mL) was added K₂CO₃ (210 mg, 1.52 mmol) and CbzCl (194 mg, 1.14 mmol, 162 uL, CAS #501-53-1). The mixture was stirred at 25° C. for 16 hours. On completion, the mixture was diluted with H₂O (5 mL), and extracted with EA (3×20 mL). The combined organic layer was washed with brine (3×20 mL), dried with anhydrous Na₂SO₄, then filtered and the filtrate was concentrated in vacuo. The residue was purified by column chromatography (SiO₂, Petroleum ether/Ethyl acetate=100/1 to 5/1) (Rf=0.35, PE:EA=2:1) to give the title compound (1.50 g, 396% yield) as a colorless oily liquid ¹H NMR (400 MHz, CDCl₃) δ7.27 (d, J=3.6 Hz, 5H), 7.18 (d, J=1.2 Hz, 1H), 5.05 (s, 2H), 4.65-4.50 (m, 1H), 3.99-3.91 (m, 1H), 3.37 (s, 4H), 2.91 (s, 3H), 2.22 (td, J=4.0, 1.6 Hz, 2H), 1.99 (s, 2H), 1.36 (s, 9H). LC-MS (ESI⁺) m/z 379.1 (M+H)⁺.

### Step 6—Benzyl N-[2-(3-aminocyclobutoxy)ethyl]-N-methyl-carbamate

To a solution of benzyl N-[2-[3-(tert-butoxycarbonylamino)cyclobutoxy]ethyl]-N-methyl-carbamate (500 mg, 1.32 mmol) in DCM (10 mL) was added HCl/dioxane (4 M, 5 mL). The mixture was stirred at 25° C. for 1 hour. On completion, the mixture was concentrated in vacuo to give the title compound (367 mg, 88% yield, HCl) as a colorless oily liquid. ¹H NMR (400 MHz, DMSO-d₆) δ 7.15 (d, J=6.8 Hz, 1H), 4.57 (t, J=5.4 Hz, 1H), 4.07-3.84 (m, 2H), 3.65-3.56 (m, 1H), 3.46 (q, J=5.2 Hz, 2H), 3.30-3.21 (m, 2H), 2.20-1.95 (m, 4H), 1.37 (s, 9H). LC-MS (ESI⁺) m/z 279.1 (M+H)⁺.

### 2-(2,6-Dioxo-3-piperidyl)-4-[[3-[2-(methylamino)ethoxy]cyclobutyl]amino]isoindoline-1,3-dione (Intermediate CCE)

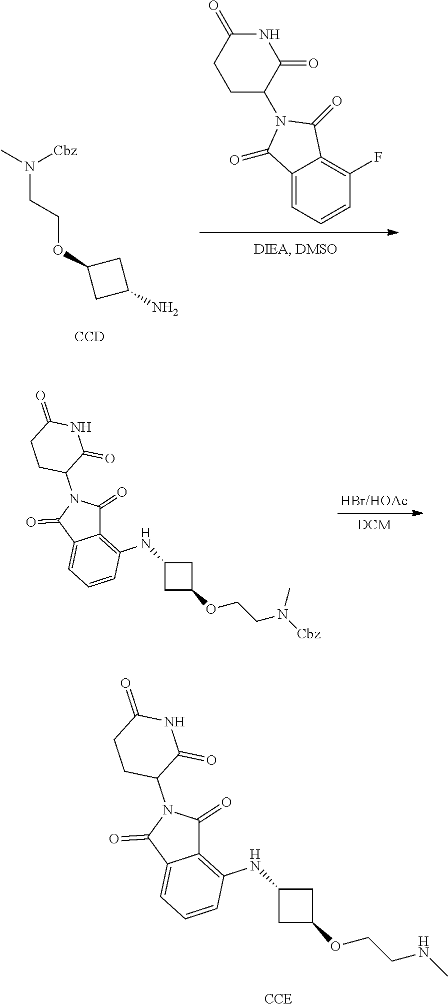 [SMILES: CN(CCO[C@H]1C[C@H](N)C1)C(=O)OCc1ccccc1 ; O=C1CCC(N2C(=O)c3cccc(F)c3C2=O)C(=O)N1 ; "CCD" ; CN(CCO[C@H]1C[C@H](Nc2cccc3c2C(=O)N(C2CCC(=O)NC2=O)C3=O)C1)C(=O)OCc1ccccc1 ; CNCCO[C@H]1C[C@H](Nc2cccc3c2C(=O)N(C2CCC(=O)NC2=O)C3=O)C1]

### Step 1—Benzyl N-[2-[3-[[2-(2,6-dioxo-3-piperidyl)-1,3-dioxo-isoindolin-4-yl]amino]cyclobutoxy]ethyl]-N-methyl-carbamate

To a solution of benzyl N-[2-(3-aminocyclobutoxy)ethyl]-N-methyl-carbamate (230 mg, 732 umol, HCl, Intermediate CCD) and 2-(2,6-dioxo-3-piperidyl)-4-fluoro-isoindoline-1,3-dione (168 mg, 610 umol) in DMSO (2 mL) was added DIEA (394 mg, 3.05 mmol, 531 uL). The mixture was then stirred at 130° C. for 3 hours. On completion, the mixture was diluted with H₂O (0.5 mL), and extracted with EA (3×5 mL). The combined organic layer was washed with brine (3×5 mL), dried with anhydrous Na₂SO₄, then filtered and the filtrate was concentrated in vacuo. The mixture was purified with pre-HPLC (column: Welch Xtimate C18 150*25 mm*5 um; mobile phase: [water(FA)-ACN]; B %: 42%-72%, 10 min) to give the title compound (170 mg, 52% yield) as a brown solid. ¹H NMR (400 MHz, DMSO-d₆) δ 11.10 (s, 1H), 7.59 (t, J=7.8 Hz, 1H), 7.38-7.26 (m, 5H), 7.09 (d, J=7.2 Hz, 1H), 6.94-6.84 (m, 1H), 6.49 (s, 1H), 5.07 (s, 2H), 4.13 (s, 2H), 3.42 (s, 5H), 2.91 (d, J=17.6 Hz, 4H), 2.36-2.30 (m, 2H), 2.21 (d, J=3.2 Hz, 2H), 2.09-1.98 (m, 1H). LC-MS (ESI⁺) m/z 535.3 (M+H)⁺.

### Step 2—2-(2,6-Dioxo-3-piperidyl)-4-[[3-[2-(methylamino)ethoxy]cyclobutyl]amino]isoindoline-1,3-dione

To a solution of benzyl N-[2-[3-[[2-(2,6-dioxo-3-piperidyl)-1,3-dioxo-isoindolin-4-yl]amino]cyclobutoxy]ethyl]-N-methyl-carbamate (60 mg, 112 umol) in DCM (1 mL) was added HBr/HOAc (1.83 g, 6.80 mmol, 1.23 mL, 30% solution). The mixture was stirred at 25° C. for 1 hour. On completion, the mixture was concentrated in vacuo to give the title compound (44 mg, 810% yield, HBr) as a brown solid. LC-MS (ESI⁺) m/z 401.1 (M+H)⁺.

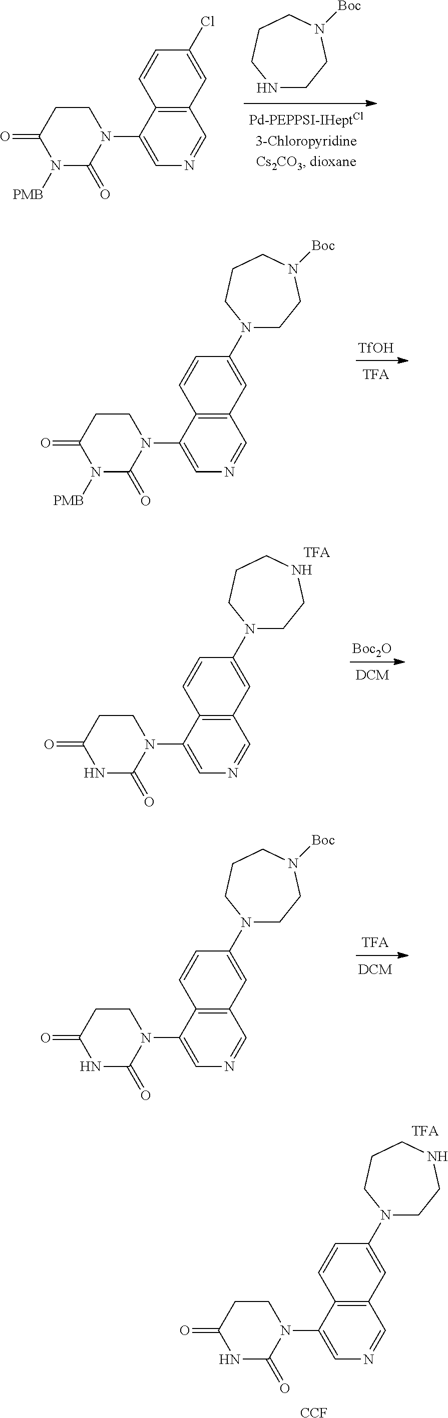

### 1-[7-(1,4-Diazepan-1-yl)-4-isoquinolyl]hexahydropyrimidine-2,4-dione (Intermediate CCF)

### Step 1—Tert-butyl 4-[4-[3-[(4-methoxyphenyl)methyl]-2,4-dioxo-hexahydropyrimidin-1-yl]-7-isoquinolyl]-1,4-diazepane-1-carboxylate

A mixture of 1-(7-chloro-4-isoquinolyl)-3-[(4-methoxyphenyl)methyl]hexahydropyrimidine-2,4-dione (300 mg, 757 umol, synthesized via Steps 1-2 of Intermediate BRX), tert-butyl 1,4-diazepane-1-carboxylate (166 mg, 833 umol, CAS #112275-50-0), Cs₂CO₃ (493 mg, 1.52 mmol), Pd-PEPPSI-IHeptCl₃-Chloropyridine (73.6 mg, 75.7 umol) in dioxane (5 mL) was degassed and purged with N₂ three times, and then the mixture was stirred at 80° C. for 48 hrs under N₂ atmosphere. On completion, filtered and concentrated under reduced pressure to give a residue. The residue was purified by column chromatography (SiO₂, PE:EA=1:1) to give the title compound (388 mg, 78% yield) as yellow solid. ¹H NMR (400 MHz, DMSO-d₆) δ 9.01 (s, 1H), 8.16 (s, 1H), 7.69 (d, J=9.2 Hz, 1H), 7.45 (d, J=9.2 Hz, 1H), 7.31-7.19 (m, 3H), 6.88 (d, J=8.4 Hz, 2H), 4.83 (s, 2H), 4.03 (q, J=7.1 Hz, 1H), 3.91-3.80 (m, 1H), 3.73 (s, 3H), 3.69-3.52 (m, 4H), 3.33 (s, 2H), 3.26-2.90 (m, 4H), 2.01-1.89 (m, 2H), 1.30-1.05 (m, 9H).

### Step 2—1-[7-(1,4-Diazepan-1-yl)-4-isoquinolyl]hexahydropyrimidine-2,4-dione

To a solution of tert-butyl 4-[4-[3-[(4-methoxyphenyl)methyl]-2,4-dioxo-hexahydropyrimidin-1-yl]-7-isoquinolyl]-1,4-diazepane-1-carboxylate (380 mg, 678 umol) in TFA (2 mL) was added TfOH (1.02 g, 6.79 mmol). The mixture was stirred at 60° C. for 3 hrs. On completion, the mixture was concentrated in vacuo to give the title compound (220 mg, 95% yield) as a black oil. LC-MS (ESI+) m/z 340.2 (M+H)⁺.

### Step 3—Tert-butyl 4-[4-(2,4-dioxohexahydropyrimidin-1-yl)-7-isoquinolyl]-1,4-diazepane-1-carboxylate

To a solution of 1-[7-(1,4-diazepan-1-yl)-4-isoquinolyl]hexahydropyrimidine-2,4-dione (220 mg, 648 umol) in DCM (4 mL) was added (Boc)₂O (282 mg, 1.30 mmol) and TEA (196 mg, 1.94 mmol). The mixture was then stirred at 25° C. for 16 hrs. On completion, filtered and concentrated under reduced pressure to give a residue. The residue was purified by reverse phase (0.1% FA condition) to give the title compound (230 mg, 80% yield) as brown solid. LC-MS (ESI+) m/z 440.4 (M+H)⁺.

### Step 4—1-[7-(1,4-Diazepan-1-yl)-4-isoquinolyl]hexahydropyrimidine-2,4-dione

To a solution of tert-butyl 4-[4-(2,4-dioxohexahydropyrimidin-1-yl)-7-isoquinolyl]-1,4-diazepane-1-carboxylate (88.0 mg, 200 umol) in DCM (2 mL) was added TFA (228 mg, 2.00 mmol). The mixture was then stirred at 25° C. for 0.5 hr. On completion, the mixture was concentrated in vacuo to give the title compound (85.0 mg, 93% yield) as a black brown oil. LC-MS (ESI+) m/z 340.2 (M+H)⁺.

### 1-[8-(1,2,3,6-Tetrahydropyridin-4-yl)-4-isoquinolyl]hexahydropyrimidine-2,4-dione (Intermediate CCG)

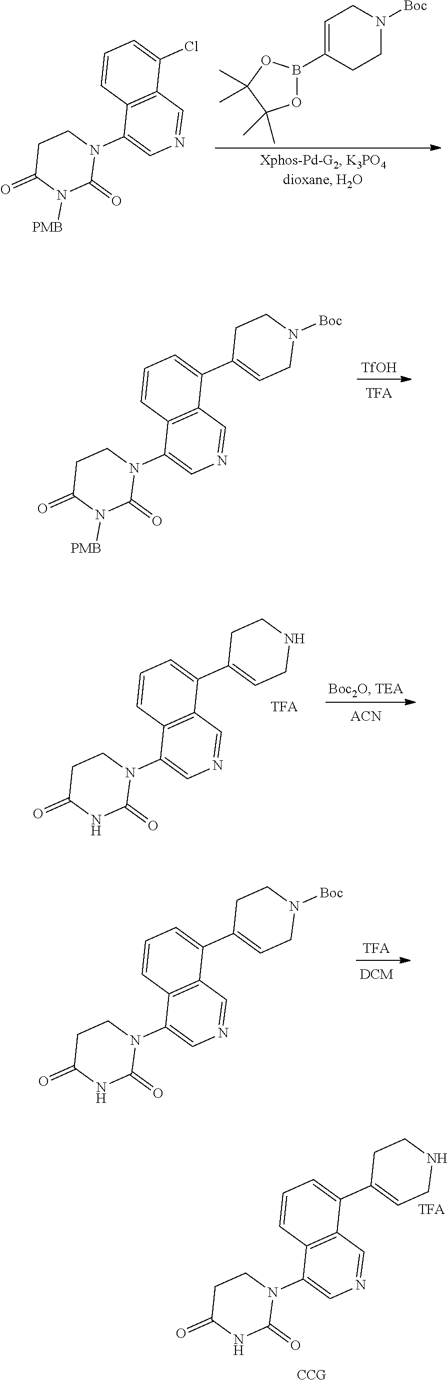

### Step 1—Tert-butyl 4-[4-[3-[(4-methoxyphenyl)methyl]-2,4-dioxo-hexahydropyrimidin-1-yl]-8-isoquinolyl]-3,6-dihydro-2H-pyridine-1-carboxylate

A mixture of 1-(8-chloro-4-isoquinolyl)-3-[(4-methoxyphenyl)methyl]hexahydropyrimidine-2,4-dione (250 mg, 631 umol, synthesized via Steps 1-2 of Intermediate BSL), tert-butyl 4-(4,4,5,5-tetramethyl-1,3,2-dioxaborolan-2-yl)-3,6-dihydro-2H-pyridine-1-carboxylate (253 mg, 821 umol, CAS #286961-14-6), Xphos-Pd-G2 (49.6 mg, 63.1 umol) and K₃PO₄ (402 mg, 1.89 mmol) in dioxane (3 mL) and H₂O (0.06 mL) was stirred at 80° C. for 12 hrs under N₂. On completion, the mixture was filtered and concentrated to give a residue. The residue was purified by reversed phase flash (0.1% FA condition) to give the title compound (340 mg, 88% yield) as a yellow solid. ¹H NMR (400 MHz, DMSO-d₆) δ 9.33 (s, 1H), 8.57 (s, 1H), 7.92-7.82 (m, 1H), 7.82-7.72 (m, 1H), 7.54 (d, J=6.8 Hz, 1H), 7.26 (d, J=8.4 Hz, 2H), 6.88 (d, J=8.4 Hz, 2H), 5.87 (s, 1H), 4.84 (s, 2H), 4.09 (s, 2H), 4.06-3.89 (m, 2H), 3.81-3.74 (m, 2H), 3.73 (s, 3H), 3.68 (t, J=5.2 Hz, 2H), 3.15 (d, J=9.6 Hz, 1H), 3.02-2.92 (m, 1H), 1.46 (s, 9H); LC-MS (ESI+) m/z 543.3 (M+H)⁺.

### Step 2—1-[8-(1,2,3,6-Tetrahydropyridin-4-yl)-4-isoquinolyl]hexahydropyrimidine-2,4-dione

To mixture of tert-butyl 4-[4-[3-[(4-methoxyphenyl)methyl]-2,4-dioxo-hexahydropyrimidin-1-yl]-8-isoquinolyl]-3,6-dihydro-2H-pyridine-1-carboxylate (240 mg, 442 umol) in TFA (5 mL) was added TfOH (1 mL). The mixture was stirred at 70° C. for 1 hr. On completion, the mixture was filtered and concentrated to give the title compound (193 mg, 99% yield, TFA salt) as a brown oil.

### Step 3—Tert-butyl 4-[4-(2,4-dioxohexahydropyrimidin-1-yl)-8-isoquinolyl]-3,6-dihydro-2H-pyridine-1-carboxylate

To a mixture of 1-[8-(1,2,3,6-tetrahydropyridin-4-yl)-4-isoquinolyl]hexahydropyrimidine-2,4-dione (193 mg, 442 umol, TFA salt) in ACN (3 mL) was added TEA (44.7 mg, 442 umol) until the pH-7-8 at 0° C., then Boc₂O (144 mg, 663 umol) in ACN (1 mL) was added. The mixture was stirred at 25° C. for 12 hrs. On completion, the mixture was filtered and concentrated to give a residue. The residue was diluted with water (30 mL) and extracted with EA (3×20 mL). The combined organic layer was dried over Na₂SO₄, filtered and concentrated in vacuo to give the title compound (186 mg, 76% yield) as a brown oil. LC-MS (ESI+) m/z 423.2 (M+H)⁺.

### Step 4—1-[8-(1,2,3,6-Tetrahydropyridin-4-yl)-4-isoquinolyl]hexahydropyrimidine-2,4-dione

To a mixture of tert-butyl 4-[4-(2,4-dioxohexahydropyrimidin-1-yl)-8-isoquinolyl]-3,6-dihydro-2H-pyridine-1-carboxylate (90.0 mg, 213 umol) in DCM (2 mL) was added TFA (1 mL). The mixture was stirred at 25° C. for 0.5 hr. On completion, the mixture was filtered and concentrated to give the title compound (92.9 mg, 100% yield, TFA salt) as a brown oil. LC-MS (ESI+) m/z 323.1 (M+H)⁺.

### N-[6-chloro-2-(4-formylcyclohexyl)indazol-5-yl]-6-(trifluoromethyl)pyridine-2-carboxamide (Intermediate CCH)

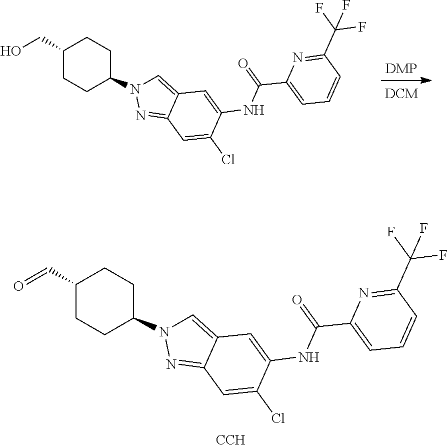 [SMILES: O=C(Nc1cc2cn([C@H]3CC[C@H](CO)CC3)nc2cc1Cl)c1cccc(C(F)(F)F)n1 ; O=C[C@H]1CC[C@H](n2cc3cc(NC(=O)c4cccc(C(F)(F)F)n4)c(Cl)cc3n2)CC1 ; "CCH"]

To a solution of N-[6-chloro-2-[4-(hydroxymethyl)cyclohexyl]indazol-5-yl]-6-(trifluoromethyl) pyridine-2-carboxamide (100 mg, 220 umol, synthesized via Steps 1-3 of Intermediate BPQ) in DCM (2 mL) was added DMP (121 mg, 287 umol). The reaction mixture was then stirred at 25° C. for 2 hours. On completion, the reaction mixture was quenched with saturated Na₂S₂O₃ (10 mL) and saturated NaHCO₃ (10 mL) at 25° C. Then, the mixture was stirred for 15 minutes then extracted with DCM (30 mL×2). The combined organic layers were washed with saturated NaCl solution (15 mL×2), dried over Na₂SO₄, filtered and concentrated in vacuo to give the title compound (98 mg, 98.4% yield) as yellow solid. LC-MS (ESI+) m/z 451.1 (M+H)⁺.

### 1-[7-[4-(Methylamino)-1-piperidyl]-4-isoquinolyl]hexahydropyrimidine-2,4-dione (Intermediate CCI)

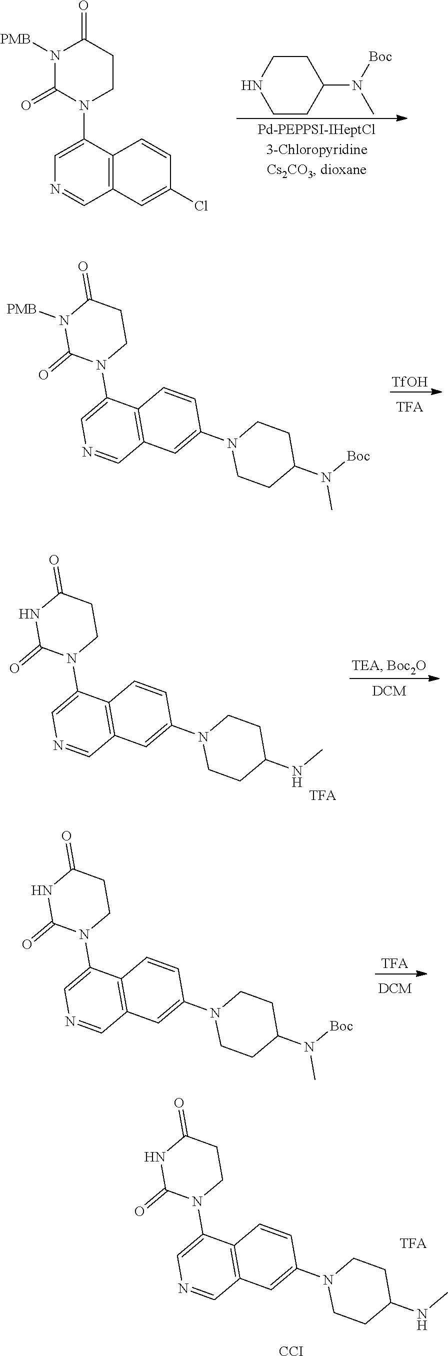

### Step 1—Tert-butyl N-[1-[4-[3-[(4-methoxyphenyl)methyl]-2,4-dioxo-hexahydropyrimidin-1-yl]-7-isoquinolyl]-4-piperidyl]-N-methyl-carbamate

To a solution of 1-(7-chloro-4-isoquinolyl)-3-[(4-methoxyphenyl)methyl]hexahydropyrimidine-2,4-dione (500 mg, 1.26 mmol, synthesized via Steps 1-2 of Intermediate BRX), tert-butyl N-methyl-N-(4-piperidyl)carbamate (270 mg, 1.26 mmol, CAS #108612-54-0), Pd-PEPPSI-IHEPTCl 3-Chloropyridine (122 mg, 126 umol) and Cs₂CO₃ (82.0 mg, 2.53 mmol) in dioxane (5 mL). The reaction mixture was then stirred at 80° C. for 16 hrs under N₂. On completion, the reaction mixture was filtered and the filtrate was concentrated in vacuo to give a residue. The residue was purified by reverse phase (0.1% FA condition) to give the title compound (200 mg, 27% yield) as a white solid. LC-MS (ESI+) m/z 574.2 (M+H)⁺.

### Step 2—1-[7-[4-(Methylamino)-1-piperidyl]-4-isoquinolyl]hexahydropyrimidine-2,4-dione

To a solution of tert-butyl N-[1-[4-[3-[(4-methoxyphenyl)methyl]-2,4-dioxo-hexahydropyrimidin-1-yl]-7-isoquinolyl]-4-piperidyl]-N-methyl-carbamate (198 mg, 345 umol) and in TFA (2 mL) was added TfOH (609 mg, 4.06 mmol). Then the reaction mixture was stirred at 70° C. for 2 hrs. On completion, the reaction mixture was concentrated in vacuo to give the title compound (150 mg, 92% yield, TFA salt) as a brown oil. LC-MS (ESI+) m/z 354.1 (M+H)⁺.

### Step 3—Tert-butyl N-[i-[4-(2,4-dioxohexahydropyrimidin-1-yl)-7-isoquinolyl]-4-piperidyl]-N-methyl-carbamate

To a solution of 1-[7-[4-(methylamino)-1-piperidyl]-4-isoquinolyl]hexahydropyrimidine-2,4-dione (150 mg, 424 umol) in DCM (2 mL) was added TEA (42.9 mg, 424 umol), then Boc₂O (92.6 mg, 424 umol) was added. The reaction was then stirred at 25° C. for 3 hrs. On completion, the reaction mixture was filtered. Then the mixture was diluted with H₂O (30 mL) and extracted with DCM (2×50 mL). The combined organic layers were dried over Na₂SO₄, filtered and concentrated in vacuo to give the title compound (170 mg, 88% yield) as a yellow solid. LC-MS (ESI+) m/z 454.2 (M+H)⁺.

### Step 4—1-[7-[4-(Methylamino)-1-piperidyl]-4-isoquinolyl]hexahydropyrimidine-2,4-dione

To a solution of tert-butyl N-[1-[4-(2,4-dioxohexahydropyrimidin-1-yl)-7-isoquinolyl]-4-piperidyl]-N-methyl-carbamate (80.3 mg, 177 umol) in DCM (1 mL) was added TFA (616 mg, 5.4 mmol). The mixture was then stirred at 25° C. for 2 hrs. On completion, the reaction mixture was concentrated in vacuo to give the title compound (80.0 mg, 96% yield, TFA salt) as a brown solid. LC-MS (ESI+) m/z 354.2 (M+H)⁺.

### 3-[4-[4-[(4-Aminocyclohexyl)methyl]piperazin-1-yl]-3-methyl-2-oxo-benzimidazol-1-yl]piperidine-2,6-dione (Intermediate CCJ)

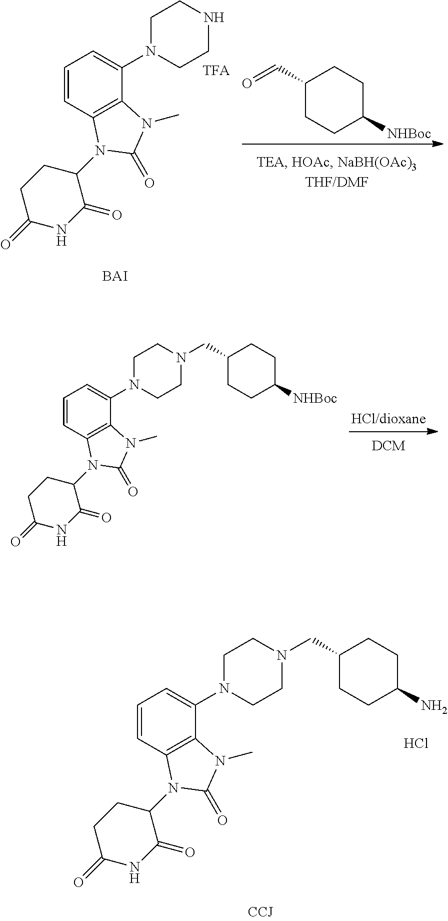 [SMILES: Cn1c(=O)n(C2CCC(=O)NC2=O)c2cccc(N3CCNCC3)c21 ; CC(C)(C)OC(=O)N[C@H]1CC[C@H](C=O)CC1 ; Cn1c(=O)n(C2CCC(=O)NC2=O)c2cccc(N3CCN(C[C@H]4CC[C@H](NC(=O)OC(C)(C)C)CC4)CC3)c21 ; Cn1c(=O)n(C2CCC(=O)NC2=O)c2cccc(N3CCN(C[C@H]4CC[C@H](N)CC4)CC3)c21 ; Cl]

### Step 1—Tert-butyl N-[4-[[4-[1-(2,6-dioxo-3-piperidyl)-3-methyl-2-oxo-benzimidazol-4-yl]piperazin-1-yl]methyl]cyclohexyl]carbamate

To a solution of 3-(3-methyl-2-oxo-4-piperazin-1-yl-benzimidazol-1-yl)piperidine-2,6-dione (300 mg, 655 umol, TFA, Intermediate BAI) in THF (5 mL) and DMF (5 mL) was added TEA (66.3 mg, 655 umol), then the reaction mixture was stirred at −10° C. for 5 mins. Next, HOAc (78.7 mg, 1.31 mmol) and tert-butyl N-(4-formylcyclohexyl)carbamate (178 mg, 787 umol, CAS #181308-57-6) were added then the mixture was stirred at −10° C. for 25 mins. Finally, NaBH(OAc)₃ (208 mg, 983 umol) was added to the mixture, and the reaction mixture was stirred at −10° C. for 1 hr. On completion, the reaction mixture was quenched with H₂O (0.5 mL) and then concentrated in vacuo to give a residue. The residue was purified by reverse phase (0.1% FA condition) to give the title compound (270 mg, 74% yield) as a white solid. ¹H NMR (400 MHz, DMSO-d₆) δ 11.10 (s, 1H), 7.05-6.98 (m, 1H), 6.97-6.86 (m, 2H), 6.74 (d, J=7.6 Hz, 1H), 5.36 (dd, J=5.2, 12.4 Hz, 1H), 3.63 (s, 3H), 3.23-2.98 (m, 6H), 2.96-2.81 (m, 2H), 2.76-2.57 (m, 3H), 2.56-2.52 (m, 3H), 2.06-1.93 (m, 1H), 1.86-1.73 (m, 4H), 1.65-1.46 (m, 1H), 1.38 (s, 9H), 1.24-1.09 (m, 2H), 1.07-0.87 (m, J=9.6 Hz, 2H); LC-MS (ESI⁺) m/z 555.3 (M+H)⁺.

### Step 2—3-[4-[4-[(4-Aminocyclohexyl)methyl]piperazin-1-yl]-3-methyl-2-oxo-benzimidazol-1-yl] piperidine-2,6-dione

To a solution of tert-butyl N-[4-[[4-[1-(2,6-dioxo-3-piperidyl)-3-methyl-2-oxo-benzimidazol-4-yl]piperazin-1-yl]methyl]cyclohexyl]carbamate (50.0 mg, 90.1 umol) in DCM (1 mL) was added HCl/dioxane (4 M, 500 uL), then the reaction mixture was stirred at 25° C. for 30 mins. On completion, the reaction mixture was concentrated in vacuo to give the title compound (40.0 mg, 90% yield, HCl salt) as a white solid. LC-MS (ESI⁺) m/z 455.2 (M+H)⁺.

### 1-[8-(1,4-Diazepan-1-yl)-4-isoquinolyl]hexahydropyrimidine-2,4-dione (Intermediate CCK)

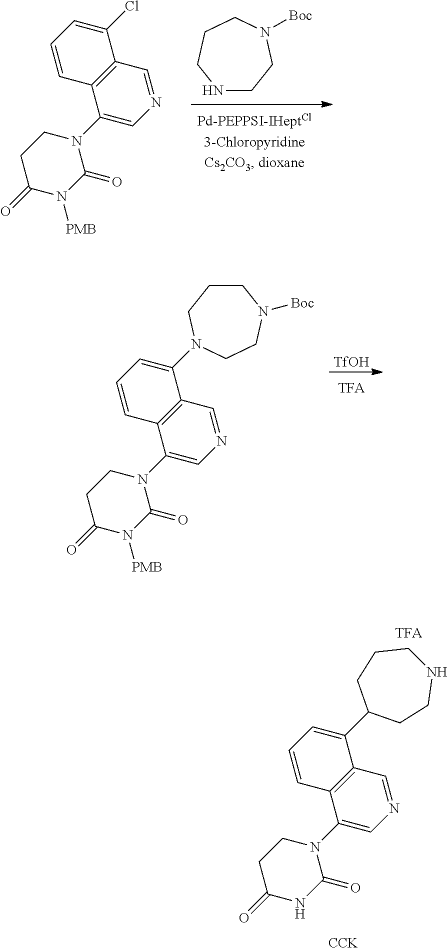

### Step 1—Tert-butyl 4-[4-[3-[(4-methoxyphenyl) methyl]-2,4-dioxo-hexahydropyrimidin-1-yl]-8-isoquinolyl]-1,4-diazepane-1-carboxylate

A mixture of 1-(8-chloro-4-isoquinolyl)-3-[(4-methoxyphenyl)methyl]hexahydropyrimidine-2,4-dione, (600 mg, 1.52 mmol, Steps 1-2 of Intermediate BSL) tert-butyl 1,4-diazepane-1-carboxylate (303 mg, 1.52 mmol, CAS #112275-50-0), PD-PEPPSI-IHeptCl³-Chloropyridine (29.4 mg, 30.3 umol), and Cs₂CO₃ (987 mg, 3.03 mmol) in dioxane (2 mL) was degassed and purged with N₂ three times. Then the mixture was stirred at 80° C. for 5 hrs under N₂ atmosphere. On completion, the reaction mixture was diluted with water (20 mL) and extracted with EA (40 mL×2). The combined organic layers were washed with NaCl (10 mL), dried over Na₂SO₄, filtered and concentrated in vacuo to give a residue. The residue was purified by reverse phase (FA condition) to give the title compound (600 mg, 70% yield) as a white solid. ¹H NMR (400 MHz, DMSO-d₆) δ 9.49 (s, 1H), 8.53 (s, 1H), 7.72-7.63 (m, 1H), 7.53 (d, J=8.4 Hz, 1H), 7.35 (d, J=8.0 Hz, 1H), 7.25 (d, J=8.4 Hz, 2H), 6.92-6.83 (m, 2H), 4.83 (s, 2H), 3.90 (m, 1H), 3.77-3.69 (m, 4H), 3.68-3.61 (m, 2H), 3.60-3.52 (m, 2H), 3.31 (s, 2H), 3.26 (s, 2H), 3.18-3.06 (m, 1H), 2.96 (m, 1H), 2.00 (d, J=3.6 Hz, 2H), 1.45 (d, J=4.0 Hz, 9H). LC-MS (ESI⁺) m/z 560.3 (M+H)⁺.

### Step 2—1-[8-(1,4-Diazepan-1-yl)-4-isoquinolyl]hexahydropyrimidine-2,4-dione

A mixture of tert-butyl 4-[4-[3-[(4-methoxyphenyl)methyl]-2,4-dioxo-hexahydropyrimidin-1-yl]-8-isoquinolyl]-1,4-diazepane-1-carboxylate (200 mg, 357 umol) in TFA (1 mL) and TfOH (0.2 mL) was degassed and purged with N₂ three times. Then the mixture was stirred at 25° C. for 2 hrs under N₂ atmosphere. On completion, the reaction mixture was concentrated in vacuo to give the title compound (162 mg, 99% yield, TFA) as a black oil. LC-MS (ESI⁺) m/z 340.2 (M+H)⁺.

### 1-[8-[(3S)-3-methylpiperazin-1-yl]-4-isoquinolyl]hexahydropyrimidine-2,4-dione (Intermediate CCL)

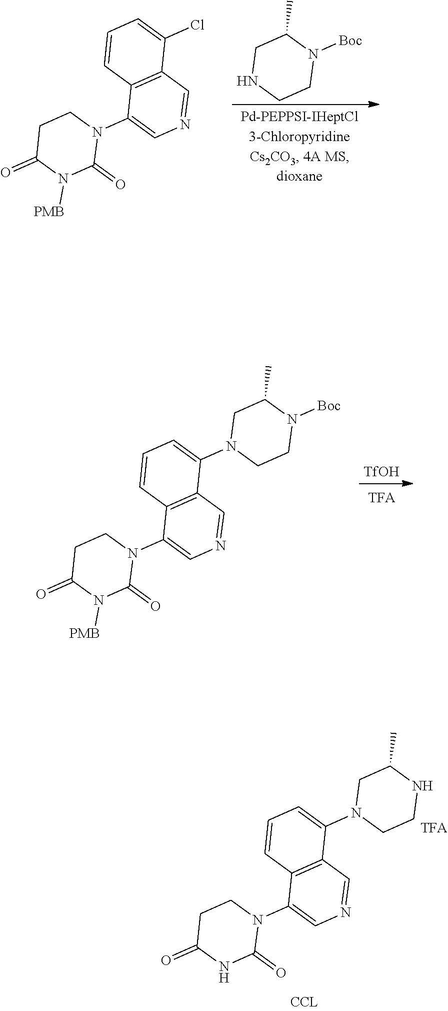

### Step 1—Tert-butyl (2S)-4-[4-[3-[(4-methoxyphenyl)methyl]-2,4-dioxo-hexahydropyrimidin-1-yl]-8-isoquinolyl]-2-methyl-piperazine-1-carboxylate

A mixture of 1-(8-chloro-4-isoquinolyl)-3-[(4-methoxyphenyl)methyl]hexahydropyrimidine-2,4-dione (600 mg, 1.52 mmol, Steps 1-2 of Intermediate BSL), tert-butyl (2S)-2-methylpiperazine-1-carboxylate (333 mg, 1.67 mmol, CAS #169447-70-5), PD-PEPPSI-IHeptCl 3-Chloropyridine (147 mg, 151 umol), Cs₂CO₃ (987 mg, 3.03 mmol) and 4 Å molecular sieves (20 mg) in dioxane (6 mL) was stirred at 100° C. for 12 hrs. On completion, the reaction mixture was filtered and filtrate was concentrated in vacuo to give a residue. The residue was diluted with water (20 mL) and extracted with EA (10 mL×3). The combined organic layer was dried over Na₂SO₄, filtered and concentrated in vacuo to give a residue. The residue was purified by reverse phase (0.1% FA condition) to give the title compound (480 mg, 57% yield) as a white solid. ¹H NMR (400 MHz, DMSO-d₆) δ 9.55 (s, 1H), 8.56 (s, 1H), 7.77-7.67 (m, 1H), 7.59 (d, J=8.4 Hz, 1H), 7.33-7.21 (m, 3H), 6.94-6.81 (m, 2H), 4.83 (s, 2H), 4.31 (d, J=2.4 Hz, 1H), 3.96-3.85 (m, 2H), 3.73 (s, 4H), 3.52-3.32 (m, 2H), 3.26-3.09 (m, 2H), 3.01-2.92 (m, 2H), 2.78 (q, J=3.2, 11.4 Hz, 1H), 1.45 (s, 9H), 1.42 (s, 3H); LC-MS (ESI⁺) m/z 560.3 (M+H)⁺.

### Step 2—1-[8-[(3S)-3-methylpiperazin-1-yl]-4-isoquinolyl]hexahydropyrimidine-2,4-dione

To a solution of tert-butyl (2S)-4-[4-[3-[(4-methoxyphenyl)methyl]-2,4-dioxo-hexahydropyrimidin-1-yl]-8-isoquinolyl]-2-methyl-piperazine-1-carboxylate (200 mg, 357 umol) in TFA (2 mL) was added TfOH (340 mg, 2.27 mmol), then the mixture was stirred at 60° C. for 2 hrs. On completion, the reaction mixture was concentrated in vacuo to give the title compound (650 mg, TFA, 95% yield) as a yellow oil. LC-MS (ESI⁺) m/z 340.3 (M+H)⁺.

### 6-(trifluoromethyl)pyridine-2-carboxylic acid (CAS #131747-42-7) (Intermediate CCM)

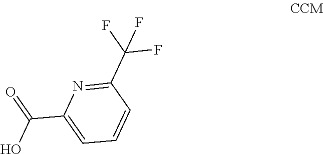 [SMILES: O=C(O)c1cccc(C(F)(F)F)n1]

### Tert-butyl N-(5-formyl-2-methoxy-4-nitro-phenyl)carbamate (Intermediate CCN)

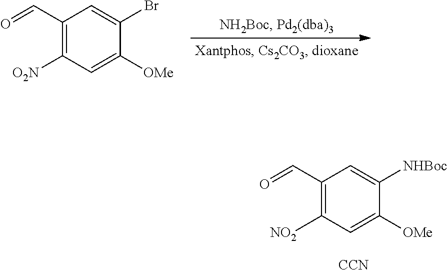

To a mixture of 5-bromo-4-methoxy-2-nitro-benzaldehyde (4.7 g, 18.0 mmol, synthesized via Steps 1-2 of Intermediate ATE) and tert-butyl carbamate (2.54 g, 21.6 mmol, CAS #4248-19-5) in dioxane (100 mL) was added Pd₂(dba)₃ (1.66 g, 1.81 mmol), Xantphos (2.09 g, 3.61 mmol) and Cs₂CO₃ (17.6 g, 54.2 mmol). The reaction mixture was then stirred at 90° C. for 12 hrs. On completion, the residue was diluted with water (30 mL) and extracted with EA (2×50 mL). The combined organic layers were washed with saturated NaCl (40 mL). The combined organic layer was dried over Na₂SO₄, filtered and concentrated in vacuo to give a residue. The residue was purified by column chromatography (SiO₂, PE:EA=20:1 to PE:EA=1:1,PE:EA=5:1, P1:Rf=0.3) then triturated with EA/PE=5/1 (50 mL) to give the title compound (4.57 g, 85% yield) as an yellow solid. ¹H NMR (400 MHz, DMSO-d₆) δ 10.17 (s, 1H), 8.75 (s, 1H), 8.40 (s, 1H), 7.72 (s, 1H), 4.00 (s, 3H), 1.49 (s, 9H); LC-MS (ESI⁺) m/z 296.9 (M+H)⁺.

### Benzyl 4-(4-aminocyclohexoxy)piperidine-1-carboxylate (Intermediate CCO)

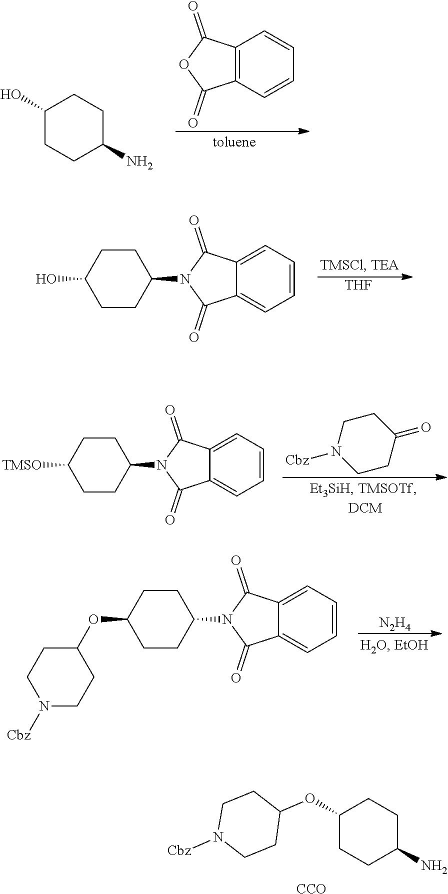

### Step 1—2-(4-Hydroxycyclohexyl)isoindoline-1,3-dione

A solution of 4-aminocyclohexanol (5.00 g, 43.41 mmol, CAS #27489-62-9) and isobenzofuran-1,3-dione (6.43 g, 43.4 mmol, CAS #85-44-9) in DMF (50 mL) and toluene (50 mL) was stirred at 120° C. for 16 hrs. On completion, the reaction mixture was concentrated in vacuo to remove toluene, then the residue was diluted with H₂O (200 mL) and filtered. The filter cake was dried in vacuo to give the title compound (10.0 g, 93% yield) as a white solid. LC-MS (ESI⁺) m/z 246.1 (M+H)⁺.

### Step 2—2-(4-Trimethylsilyloxycyclohexyl)isoindoline-1,3-dione

To a solution of 2-(4-hydroxycyclohexyl)isoindoline-1,3-dione (4 g, 16.3 mmol) in THF (50 mL) was added TEA (1.98 g, 19.5 mmol, 2.72 mL), then TMSCl (1.86 g, 17.12 mmol, 2.17 mL) was added at 0° C. dropwise. Then the reaction mixture was stirred at 25° C. for 3 hrs. On completion, the reaction mixture was diluted with H₂O (80 mL) and extracted with EA (2×40 mL). The combined organic layers were dried over Na₂SO₄, filtered and the filtrate was concentrated in vacuo to give the title compound (4.70 g, 90% yield) as a white solid. ¹H NMR (400 MHz, CDCl₃) δ 7.73-7.62 (m, 2H), 7.60-7.51 (m, 2H), 3.98 (tt, J=4.0, 12.4 Hz, 1H), 3.56 (tt, J=4.4, 10.8 Hz, 1H), 2.19 (dq, J=3.6, 13.2 Hz, 2H), 1.88-1.77 (m, 2H), 1.65-1.54 (m, 2H), 1.41-1.26 (m, 2H), 0.00 (s, 9H).

### Step 3—Benzyl 4-[4-(1,3-dioxoisoindolin-2-yl)cyclohexoxy]piperidine-1-carboxylate

To a solution of 2-(4-trimethylsilyloxycyclohexyl)isoindoline-1,3-dione (4.70 g, 14.8 mmol) and benzyl 4-oxopiperidine-1-carboxylate (3.45 g, 14.8 mmol, 2.95 mL, CAS #19099-93-5) in DCM (100 mL) was added Et₃SiH (1.89 g, 16.2 mmol, 2.60 mL) and TMSOTf (1.65 g, 7.40 mmol, 1.34 mL) at −60° C. Then the reaction mixture was stirred at 25° C. for 1 hr. On completion, the reaction mixture was diluted with H₂O (60 mL) and extracted with DCM (2×40 mL). The combined organic layers were dried over Na₂SO₄, filtered and the filtrate was concentrated under reduced pressure to give a residue. The residue was purified by reverse phase (0.1% FA condition) to give the title compound (4.30 g, 62% yield) as white solid.

¹H NMR (400 MHz, DMSO-d₆) δ 7.88-7.78 (m, 4H), 7.43-7.25 (m, 5H), 5.06 (s, 2H), 3.98 (tt, J=4.0, 12.4 Hz, 1H), 3.78-3.59 (m, 3H), 3.49-3.38 (m, 1H), 3.22-3.02 (m, 2H), 2.16 (dq, J=3.2, 12.8 Hz, 2H), 2.06-1.96 (m, 2H), 1.84-1.65 (m, 4H), 1.41-1.20 (m, 4H); LC-MS (ESI⁺) m/z 463.2 (M+H)⁺.

### Step 4—Benzyl 4-(4-aminocyclohexoxy)piperidine-1-carboxylate

To a solution of benzyl 4-[4-(1,3-dioxoisoindolin-2-yl)cyclohexoxy]piperidine-1-carboxylate (4.30 g, 9.30 mmol) in EtOH (40 mL) was added N₂H4H₂O (2.27 g, 44.3 mmol, 2.2 mL, 98% solution), then the reaction mixture was stirred at 80° C. for 2 hrs. On completion, the reaction mixture was filtered and the filtrate was concentrated in vacuo to give a residue. The residue was then diluted with DCM (30 mL), filtered and the filtrate was concentrated in vacuo to give the title compound (2.90 g, 94% yield) as colorless oil. ¹H NMR (400 MHz, CDCl₃) δ 7.62-7.28 (m, 5H), 5.13 (s, 2H), 3.91-3.75 (m, 2H), 3.64-3.53 (m, 1H), 3.39-3.28 (m, 1H), 3.22-3.13 (m, 2H), 2.78-2.67 (m, 1H), 1.99-1.89 (m, 3H), 1.88-1.84 (m, 3H), 1.83-1.72 (m, 2H), 1.61-1.45 (m, 2H), 1.39-1.25 (m, 2H), 1.23-1.08 (m, 2H).

### Tert-butyl N-[6-methoxy-2-[4-(4-piperidyloxy)cyclohexyl]indazol-5-yl]carbamate (Intermediate CCP)

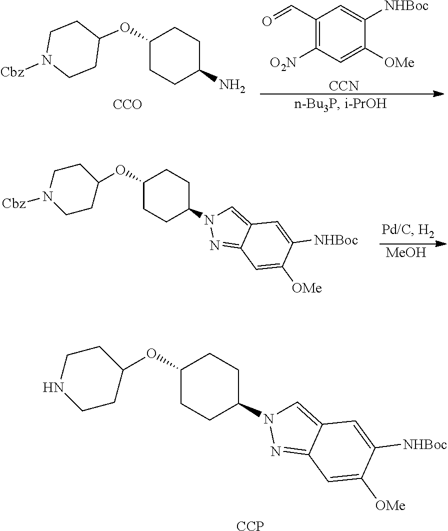

### Step 1—Benzyl 4-[4-[5-(tert-butoxycarbonylamino)-6-methoxy-indazol-2-yl]cyclohexoxy]piperidine-1-carboxylate

A solution of benzyl 4-(4-aminocyclohexoxy)piperidine-1-carboxylate (2.70 g, 8.12 mmol, Intermediate CCO) and tert-butyl N-(5-formyl-2-methoxy-4-nitro-phenyl)carbamate (2.41 g, 8.12 mmol, Intermediate CCN) in IPA (50 mL) was stirred at 80° C. for 4 hrs. Next, the mixture was cooled to 25° C., and tributylphosphane (4.93 g, 24.3 mmol, 6.01 mL) was added. Then the reaction mixture was stirred at 80° C. for 12 hrs. On completion, the reaction mixture was concentrated in vacuo to give a residue. The residue was purified by reverse phase (0.1% FA condition) to give the title compound (2.30 g, 49% yield) as a yellow solid. H NMR (400 MHz, DMSO-d₆) δ 8.16 (s, 1H), 7.90 (s, 1H), 7.77 (s, 1H), 7.46-7.25 (m, 5H), 6.98 (s, 1H), 5.07 (s, 2H), 4.43-4.30 (m, 1H), 3.84 (s, 3H), 3.77-3.68 (m, 2H), 3.67-3.58 (m, 1H), 3.53-3.45 (m, 1H), 3.21-3.06 (m, 2H), 2.11-2.00 (m, 4H), 1.98-1.85 (m, 2H), 1.84-1.74 (m, 2H), 1.46 (s, 9H), 1.42-1.27 (m, 4H); LC-MS (ESI⁺) m/z 579.6 (M+H)⁺.

### Step 2—Tert-butyl N-[6-methoxy-2-[4-(4-piperidyloxy)cyclohexyl]indazol-5-yl]carbamate

To a solution of benzyl 4-[4-[5-(tert-butoxycarbonylamino)-6-methoxy-indazol-2-yl]cyclohexoxy]piperidine-1-carboxylate (2.30 g, 3.97 mmol) in MeOH (30 mL) was added Pd/C (500 mg, 10 wt %), then the reaction mixture was stirred at 25° C. for 16 hrs under H₂ (15 psi). On completion, the mixture was filtered and the filtrate was concentrated in vacuo to give the title compound (1.75 g, 99% yield) as a yellow solid. LC-MS (ESI⁺) m/z 445.3 (M+H)⁺.

### 3-[4-[4-[4-(5-Amino-6-methoxy-indazol-2-yl)cyclohexoxy]-1-piperidyl]-3-methyl-2-oxo-benzimidazol-1-yl]piperidine-2,6-dione (Intermediate CCQ)

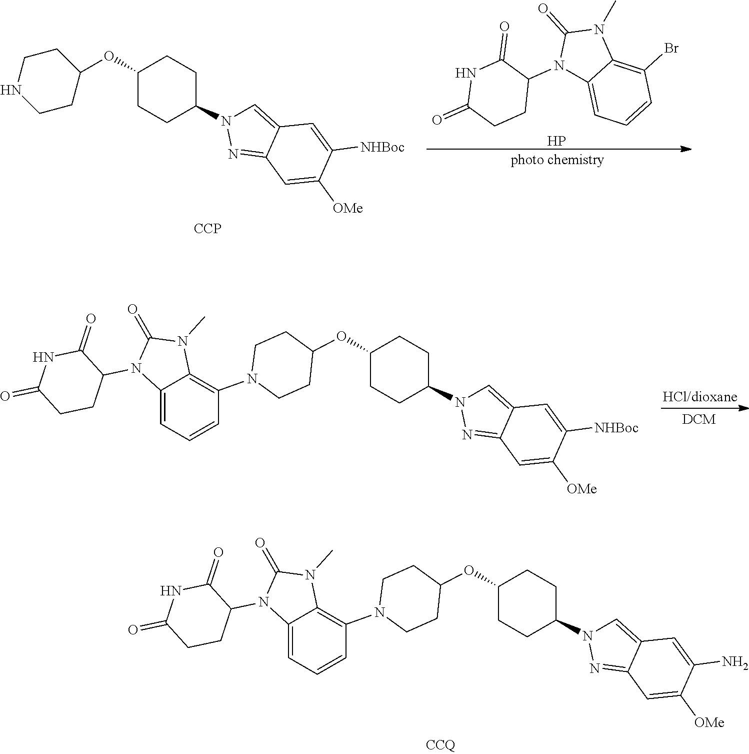

### Step 1—Tert-butyl N-[2-[4-[[1-[1-(2,6-dioxo-3-piperidyl)-3-methyl-2-oxo-benzimidazol-4-yl]-4-piperidyl]oxy]cyclohexyl]-6-methoxy-indazol-5-yl]carbamate

A mixture of 3-(4-bromo-3-methyl-2-oxo-benzimidazol-1-yl)piperidine-2,6-dione (304 mg, 899 umol, Intermediate HP), tert butyl N-[6-methoxy-2-[4-(4-piperidyloxy)cyclohexyl]indazol-5-yl]carbamate (400 mg, 8 umol, Intermediate CCP), DABCO (181 mg, 1.62 mmol), IR(PPY)₂(DTBBPY)PF₆ (16.4 mg, 18.0 umol), and NiBr₂.glyme (13.9 mg, 45.0 umol) in DMA (8 mL) was degassed three times with N₂. The reaction vial was then sealed with parafilm, placed 2 cm away from one blue LED, and irradiated at 25° C. for 14 hrs. On completion, the mixture was diluted with EA (15 mL×3) and washed with H2O (30 mL). The combined organic phase was dried over anhydrous sodium sulfate, filtered and concentrated to give a residue. The residue was purified by reverse phase (0.1% FA condition) to give the title compound (25 mg, 4% yield) as a white solid. ¹H NMR (400 MHz, DMSO-d₆) δ 11.09 (s, 1H), 8.19 (s, 1H), 7.90 (s, 1H), 7.78 (s, 1H), 7.01-6.95 (m, 2H), 6.93-6.85 (m, 2H), 5.35 (dd, J=5.2, 12.4 Hz, 1H), 4.46-4.34 (m, 1H), 3.85 (s, 3H), 3.64 (s, 3H), 3.62-3.47 (m, 2H), 3.16-2.99 (m, 2H), 2.95-2.79 (m, 2H), 2.78-2.62 (m, 3H), 2.18-2.05 (m, 5H), 2.04-1.89 (m, 5H), 1.67-1.54 (m, 2H), 1.47 (s, 12H); LC-MS (ESI⁺) m/z 702.3 (M+H)⁺.

### Step 2—3-[4-[4-[4-(5-Amino-6-methoxy-indazol-2-yl)cyclohexoxy]-1-piperidyl]-3-methyl-2-oxo-benzimidazol-1-yl]piperidine-2,6-dione

To a solution of tert-butyl N-[2-[4-[[1-[1-(2,6-dioxo-3-piperidyl)-3-methyl-2-oxo-benzimidazol-4-yl]-4-piperidyl]oxy]cyclohexyl]-6-methoxy-indazol-5-yl]carbamate (18.0 mg, 25.6 umol) in DCM (1 mL) was added HCl/dioxane (4 M, 0.5 mL). Then the reaction mixture was stirred at 25° C. for 30 mins. On completion, the reaction mixture was concentrated in vacuo to give residue to give the title compound (16 mg, 97% yield, HCl salt) as a white solid. LC-MS (ESI⁺) m/z 602.4 (M+H)⁺.

### 6-Bromo-5-methoxy-2-methylsulfonyl-1,3-benzothiazole (Intermediate BJM)

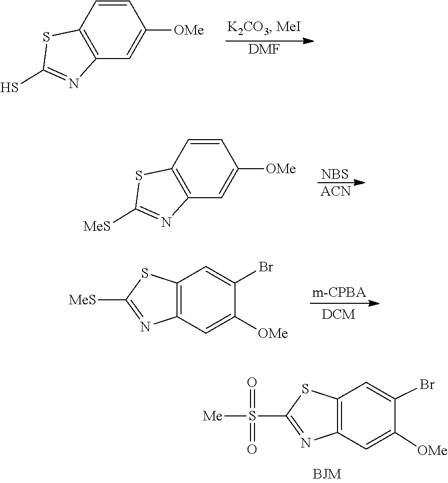

### Step 1—5-Methoxy-2-methylsulfanyl-1,3-benzothiazole

To a mixture of 5-methoxy-1,3-benzothiazole-2-thiol (3.80 g, 19.2 mmol, CAS #55690-60-3) in DMF (50.0 mL) was added K₂CO₃ (5.32 g, 38.5 mmol) and CH₃I (4.10 g, 28.8 mmol) at 0° C. The mixture was stirred at 20° C. for 0.5 hour. On completion, the mixture was poured into the water (120 mL) and the aqueous phase was extracted with ethyl acetate (2×50 mL). The combined organic phase was washed with brine (2×50 mL), dried with anhydrous Na₂SO₄, filtered and concentrated in vacuo to give the title compound (4.00 g, 98% yield) as brown oil. ¹H NMR (400 MHz, CDCl₃) δ 7.60 (d, J=8.8 Hz, 1H), 7.40 (d, J=2.4 Hz, 1H), 6.94 (dd, J=2.4, 8.8 Hz, 1H), 3.87 (s, 3H), 2.78 (s, 3H).

### Step 2—6-Bromo-5-methoxy-2-methylsulfanyl-1,3-benzothiazole

To a mixture of 5-methoxy-2-methylsulfanyl-1,3-benzothiazole (3.8 g, 17.9 mmol) in CH₃CN (40 mL) was added NBS (3.20 g, 17.9 mmol) at 0° C. The mixture was stirred at 0° C. for 0.5 hour. On completion, the mixture was concentrated at 45° C. The residue was purified by re-crystallization from EA (5 mL) and MTBA (15 mL) at 60° C. to give the title compound (2.00 g, 38% yield) as white solid. ¹H NMR (400 MHz, CDCl₃) δ 7.81 (s, 1H), 7.32 (s, 1H), 3.88 (s, 3H), 2.70 (s, 3H).

### Step 3—6-Bromo-5-methoxy-2-methylsulfonyl-1,3-benzothiazole

To a mixture of 6-bromo-5-methoxy-2-methylsulfanyl-1,3-benzothiazole (2.00 g, 6.89 mmol) in DCM (20 mL) was added m-CPBA (2.80 g, 13.7 mmol, 85% solution). The reaction mixture was stirred at 25° C. for 0.5 hour. On completion, the mixture was poured into the water (200 mL), and extracted with DCM (2×150 mL). The combined organic phase was washed with brine (2×200 mL), dried with anhydrous Na₂SO₄, filtered and concentrated in vacuo at 45° C. to give the title compound (2.70 g, 80% yield) as white solid, ¹H NMR (400 MHz, CDCl₃) δ 8.10 (s, 1H), 7.55 (s, 1H), 3.93 (s, 3H), 3.32 (s, 3H).

### [1-(6-Bromo-5-methoxy-1,3-benzothiazol-2-yl)-4-piperidyl]methanol (Intermediate BJN)

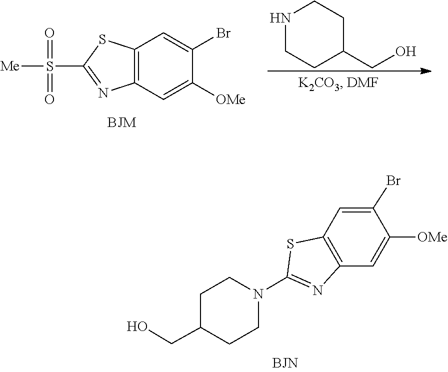 [SMILES: COc1cc2nc(S(C)(=O)=O)sc2cc1Br ; OCC1CCNCC1 ; COc1cc2nc(N3CCC(CO)CC3)sc2cc1Br]

To a solution of 6-bromo-5-methoxy-2-methylsulfonyl-1,3-benzothiazole (2.7 g, 8.38 mmol, Intermediate BJM) and 4-piperidylmethanol (1.06 g, 9.22 mmol, CAS #6457-49-4) in DMF (30 mL) was added K₂CO₃ (2.32 g, 16.7 mmol), the mixture was stirred at 60° C. for 16 hrs. On completion, the mixture was diluted with H₂O (180 mL), then extracted with EA (3×50 mL). The organic layers were washed with brine (3×50 mL) and dried over anhydrous Na₂SO₄, filtered and concentrated in vacuo. The mixture was purified by silica gel column (PE:EA=2:3) to give the title compound (1.70 g, 56% yield) as white solid. ¹H NMR (400 MHz, DMSO-d₆) δ 7.94 (s, 1H), 7.17 (s, 1H), 4.54 (t, J=5.2 Hz, 1H), 4.05-3.95 (m, 2H), 3.84 (s, 3H), 3.31-3.27 (m, 2H), 3.18-3.08 (m, 2H), 1.83-1.73 (m, 2H), 1.72-1.60 (m, 1H), 1.28-1.11 (m, 2H), LC-MS (ESI⁺) m/z 359.0 (M+H)⁺.

### N-[2-(4-formyl-1-piperidyl)-5-methoxy-1,3-benzothiazol-6-yl]-6-(trifluoromethyl)pyridine-2-carboxamide (Intermediate CCR)

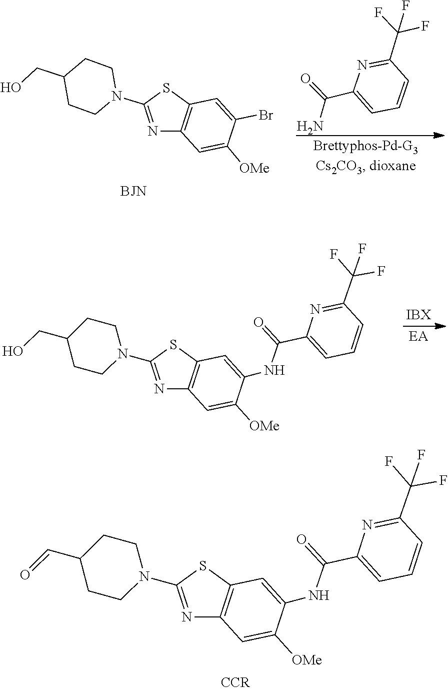 [SMILES: COc1cc2nc(N3CCC(CO)CC3)sc2cc1Br ; NC(=O)c1cccc(C(F)(F)F)n1 ; COc1cc2nc(N3CCC(CO)CC3)sc2cc1NC(=O)c1cccc(C(F)(F)F)n1 ; COc1cc2nc(N3CCC(C=O)CC3)sc2cc1NC(=O)c1cccc(C(F)(F)F)n1]

### Step 1—N-[2-[4-(hydroxymethyl)-1-piperidyl]-5-methoxy-1,3-benzothiazol-6-yl]-6-(trifluoromethyl) pyridine-2-carboxamide

To a solution of [1-(6-bromo-5-methoxy-1,3-benzothiazol-2-yl)-4-piperidyl]methanol (1.40 g, 3.92 mmol, Intermediate BJN) in dioxane (20 mL) was added 6-(trifluoromethyl)pyridine-2-carboxamide (894 mg, 4.70 mmol), 4A MS (3.92 mmol), Cs₂CO₃ (2.55 g, 7.84 mmol) and [2-(2-aminophenyl)phenyl]-methylsulfonyloxy-palladium; dicyclohexyl-[3,6-dimethoxy-2-(2,4,6-triisopropylphenyl)phenyl]phosphane (355 mg, 391 umol, CAS #22245-84-7). The mixture was then stirred at 90° C. for 12 hrs under N₂. On completion, the mixture was filtered and concentrated in vacuo. The residue was purified by column chromatography (SiO₂, Petroleum ether/Ethyl acetate=5:1 to 1:1) to give the title compound (250 mg, 12% yield) as a yellow solid. ¹H NMR (400 MHz, DMSO-d₆) δ 10.33 (s, 1H), 8.70 (s, 1H), 8.53-8.42 (m, 1H), 8.42-8.32 (m, 1H), 8.21 (dd, J=1.2, 7.6 Hz, 1H), 7.27 (s, 1H), 4.54 (t, J=5.2 Hz, 1H), 4.07-3.98 (m, 2H), 3.95 (s, 3H), 3.30-3.27 (m, 2H), 3.14 (dt, J=2.4, 12.4 Hz, 2H), 1.83-1.60 (m, 3H), 1.28-1.15 (m, 2H); LC-MS (ESI⁺) m/z 466.9 (M+H)⁺.

### Step 2—N-[2-(4-formyl-1-piperidyl)-5-methoxy-1,3-benzothiazol-6-yl]-6-(trifluoromethyl)pyridine-2-carboxamide

To a solution of N-[2-[4-(hydroxymethyl)-1-piperidyl]-5-methoxy-1,3-benzothiazol-6-yl]-6-(trifluoromethyl)pyridine-2-carboxamide (100 mg, 214 umol) in EA (1 mL) were added IBX (180 mg, 643 umol) and 4 Å molecular sieves (50.0 mg, 214 umol). The mixture was then stirred at 50° C. for 12 hrs. On completion, the mixture was filtered and concentrated to give a residue. The residue was purified by prep-TLC (SiO₂, PE:EA=1:1) to give the title compound (100 mg, 85% yield) as a yellow solid. ¹H NMR (400 MHz, DMSO-d₆) δ 10.33 (s, 1H), 9.63 (s, 1H), 8.71 (s, 1H), 8.49-8.34 (m, 2H), 8.20 (dd, J=0.8, 7.6 Hz, 1H), 7.28 (s, 1H), 3.95 (s, 3H), 3.92 (t, J=3.6 Hz, 2H), 3.31-3.26 (m, 2H), 2.72-2.61 (m, 1H), 2.03-1.94 (m, 2H), 1.66-1.53 (m, 2H); LC-MS (ESI⁺) m/z 465.0 (M+H)⁺.

### 3-(5-methoxy-3-methyl-2-oxo-4-piperazin-1-yl-benzimidazol-1-yl)piperidine-2,6-dione (Intermediate CCS)

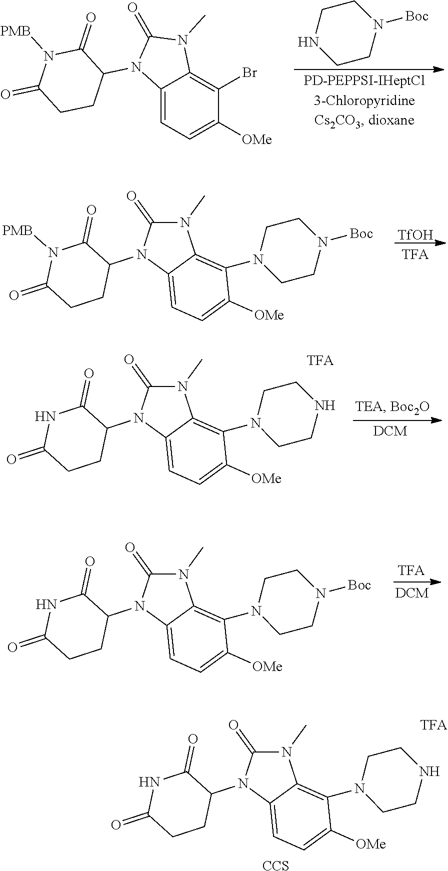

### Step 1—Tert-butyl 4-[5-methoxy-1-[1-[(4-methoxyphenyl)methyl]-2,6-dioxo-3-piperidyl]-3-methyl-2-oxo-benzimidazol-4-yl]piperazine-1-carboxylate

To a solution of 3-(4-bromo-5-methoxy-3-methyl-2-oxo-benzimidazol-1-yl)-1-[(4-methoxyphenyl)methyl]piperidine-2,6-dione (200 mg, 409 umol, synthesized via Step 1 of Intermediate BWM) and tert-butyl piperazine-1-carboxylate (114 mg, 614 umol, CAS #143238-38-4) in dioxane (5 mL) was added Cs₂CO₃ (400 mg, 1.23 mmol) and PD-PEPPSI-IHeptCl₃-Chloropyridine (39.8 mg, 40.9 umol) at 25° C. Then the reaction mixture was stirred at 100° C. for 10 hours under N₂. On completion, the reaction mixture was filtered and concentrated in vacuo. The residue was purified by prep-HPLC (column: YMC Triart C18 250*50 mm*7 um; mobile phase: [water (FA)-ACN]; B %: 60%-90%, 10 min) to give the title compound (110 mg, 15% yield) as yellow solid. ¹H NMR (400 MHz, DMSO-d₆) δ 7.20 (d, J=8.8 Hz, 2H), 6.86 (d, J=8.8 Hz, 2H), 6.78 (d, J=8.4 Hz, 1H), 6.63 (d, J=8.4 Hz, 1H), 5.46 (dd, J=5.2, 12.8 Hz, 1H), 4.85-4.71 (m, 2H), 3.92-3.79 (m, 2H), 3.76 (s, 3H), 3.72 (s, 3H), 3.61 (s, 3H), 3.31-3.25 (m, 3H), 3.10-2.97 (m, 2H), 2.86-2.77 (m, 3H), 2.75-2.64 (m, 1H), 2.06-1.97 (m, 1H), 1.43 (s, 9H).

### Step 2—3-(5-methoxy-3-methyl-2-oxo-4-piperazin-1-yl-benzimidazol-1-yl)piperidine-2,6-dione

To a solution of tert-butyl 4-[5-methoxy-1-[1-[(4-methoxyphenyl) methyl]-2,6-dioxo-3-piperidyl]-3-methyl-2-oxo-benzimidazol-4-yl]piperazine-1-carboxylate (80.0 mg, 134 umol) in TFA (1 mL) was added TfOH (340 mg, 2.27 mmol). Then the reaction mixture was stirred at 70° C. for 3 hours. On completion, the reaction mixture was concentrated in vacuo to give the title compound (65.0 mg, 98% yield, TFA) as brown oil. LC-MS (ESI⁺) m/z 374.1 (M+H)⁺.

### Step 3—Tert-butyl 4-[1-(2,6-dioxo-3-piperidyl)-5-methoxy-3-methyl-2-oxo-benzimidazol-4-yl]piperazine-1-carboxylate

To a solution of 3-(5-methoxy-3-methyl-2-oxo-4-piperazin-1-yl-benzimidazol-1-yl)piperidine-2,6-dione (80.0 mg, 164 umol, TFA) in DCM (2 mL) was added TEA (49.8 mg, 492 umol) and Boc₂O (42.9 mg, 196 umol). Then the reaction mixture was stirred at 25° C. for 5 hours. On completion, the reaction mixture was diluted with DCM (80 mL), washed with water (50 mL×4), filtered and concentrated in vacuo to give the title compound (77 mg, 99.0% yield) as brown solid. LC-MS (ESI⁺) m/z 474.3 (M+H)⁺.

### Step 4—3-(5-methoxy-3-methyl-2-oxo-4-piperazin-1-yl-benzimidazol-1-yl)piperidine-2,6-dione

To a solution of tert-butyl 4-[1-(2,6-dioxo-3-piperidyl)-5-methoxy-3-methyl-2-oxo-benzimidazol-4-yl]piperazine-1-carboxylate (50.0 mg, 105 umol) in DCM (0.7 mL) was added TFA (462 mg, 4.05 mmol), then the reaction mixture was stirred at 25° C. for 0.5 hours. On completion, the reaction mixture was concentrated in vacuo to give the title compound (50.0 mg, 97% yield, TFA) as brown soil. LC-MS (ESI⁺) m/z 374.1 (M+H)⁺.

### 1-(8-Bromo-4-isoquinolyl)hexahydropyrimidine-2,4-dione (Intermediate CCT)

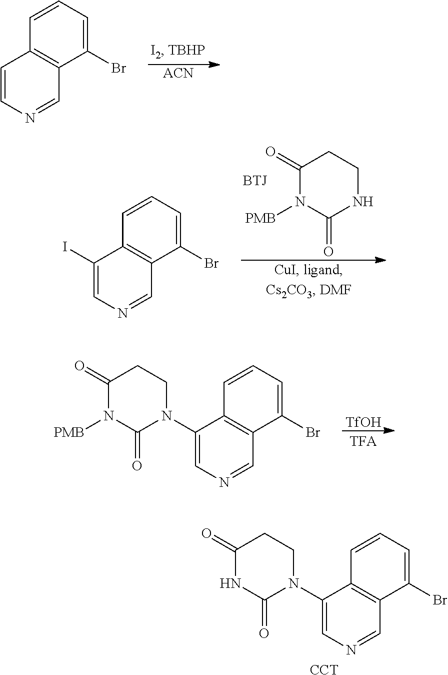

### Step 1—8-Bromo-4-iodo-isoquinoline

To a solution of 8-bromoisoquinoline (2.20 g, 10.5 mmol, CAS #63927-22-0) in DCE (50 mL) was added I₂ (5.37 g, 21.1 mmol) and TBHP (2.86 g, 31.7 mmol). The mixture was then stirred at 85° C. for 16 hrs. On completion, the mixture was quenched with Na₂S₂O₃ aqueous (25 mL) and extracted with ethyl acetate (25 mL×3). The combined organic phase was dried over anhydrous sodium sulfate, filtered and concentrated to give a residue. The residue was triturated with EA to give the crude compound (1.9 g, 53% yield) as yellow solid. ¹H NMR (400 MHz, CDCl₃) δ9.54 (s, 1H), 9.03 (s, 1H), 8.02 (d, J=8.4 Hz, 1H), 7.92 (d, J=7.6 Hz, 1H), 7.63 (dd, J=7.6, 8.4 Hz, 1H).

### Step 2—1-(8-Bromo-4-isoquinolyl)-3-[(4-methoxyphenyl)methyl]hexahydropyrimidine-2,4-dione

To a solution of 3-[(4-methoxyphenyl)methyl]hexahydropyrimidine-2,4-dione (1.77 g, 7.55 mmol, Intermediate BTJ) in DMF (20 mL) was added 8-bromo-4-iodo-isoquinoline (1.68 g, 5.03 mmol), CuI (383 mg, 2.01 mmol), Cs₂CO₃ (3.28 g, 10.0 mmol), (1R,2R)-N1,N2-dimethylcyclohexane-1,2-diamine (286 mg, 2.01 mmol) and 4 Å molecular sieves (300 mg, 2.01 mmol) under N₂. The mixture was stirred at 65° C. for 12 hrs. On completion, the mixture was filtered, and the filtrate was concentrated in vacuo. The residue was purified by column chromatography (SiO₂, Petroleum ether/Ethyl acetate=5:1 to 0:1) to give the title compound (1.2 g, 54% yield) as yellow solid. ¹H NMR (400 MHz, CDCl₃) δ 9.57 (s, 1H), 8.49 (s, 1H), 7.83 (dd, J=0.8, 7.2 Hz, 1H), 7.60-7.50 (m, 1H), 7.50-7.44 (m, 1H), 7.35 (d, J=8.4 Hz, 2H), 6.79-6.76 (m, 2H), 4.92 (s, 2H), 3.83 (ddd, J=6.4, 8.0, 12.4 Hz, 1H), 3.73 (s, 3H), 3.30-3.27 (m, 1H), 2.99-2.91 (m, 2H).

### Step 3—1-(8-Bromo-4-isoquinolyl)hexahydropyrimidine-2,4-dione

To a mixture of 1-(8-bromo-4-isoquinolyl)-3-[(4-methoxyphenyl)methyl]hexahydropyrimidine-2,4-dione (1 g, 2.27 mmol) in TFA (10 mL) was added TfOH (2 mL). The mixture was stirred at 70° C. for 12 hr. On completion, the reaction mixture was concentrated in vacuo, then diluted with EA (10 mL) and basified with TEA until the pH=8-9. The mixture was filtered and concentrated in vacuo to give the title compound (500 mg, 58% yield) as a brown solid. LC-MS (ESI+) m/z 319.7 (M+H)⁺.

### (3-bromocyclobutyl)methoxy-tert-butyl-dimethyl-silane (Intermediate CCU)

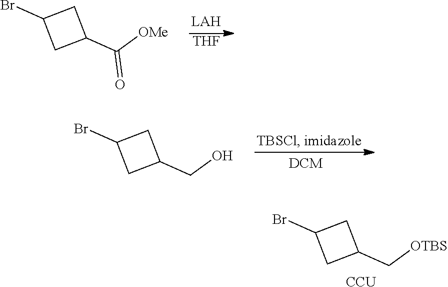

### Step 1—3-Bromocyclobutyl)methanol

To a solution of methyl 3-bromocyclobutanecarboxylate (2.00 g, 10.3 mmol, CAS #4935-00-6) in THF (30 mL) was added LAH (393 mg, 10.3 mmol) at 0° C., then the mixture was stirred at 25° C. for 1 hr. On completion, the mixture was quenched with water (0.4 mL), 15% NaOH (0.4 mL) and water (1.2 mL), and filtered to give the title compound (1.60 g, 93% yield) as yellow oil. ¹H NMR (400 MHz, CDCl₃-d) δ 4.60-4.53 (m, 1H), 3.66-3.64 (m, 2H), 2.81-2.72 (m, 1H), 2.63-2.52 (m, 4H), 1.61 (s, 1H).

### Step 2—(3-Bromocyclobutyl)methoxy-tert-butyl-dimethyl-silane

To a solution of (3-bromocyclobutyl)methanol (500 mg, 3.03 mmol) in DCM (8 mL) was added TBSCl (557 uL, 4.54 mmol) and imidazole (412 mg, 6.06 mmol), then the mixture was stirred at 25° C. for 3 hours. On completion, the mixture was quenched with NH₄Cl(10 mL) and DCM (5 mL). Then the mixture was extracted with DCM (20 mL×3), the combined organic layers was concentrated in vacuo to give the residue. The residue was purified by column chromatography to give the title compound (800 mg, 94% yield) as a colorless oil. ¹H NMR (400 MHz, CDCl₃-d) δ 4.60-4.53 (m, 1H), 3.66-3.64 (m, 2H), 2.81-2.72 (m, 1H), 2.63-2.52 (m, 4H), 1.61 (s, 1H).

### 1-[8-[3-[[Tert-butyl(dimethyl)silyl]oxymethyl]cyclobutyl]-4-isoquinolyl]hexahydropyrimidine-2,4-dione (Intermediate CCV) and 1-[8-[3-[[tert-butyl(dimethyl)silyl]oxymethyl]cyclobutyl]-4-isoquinolyl]hexahydropyrimidine-2,4-dione (Intermediate CCW)

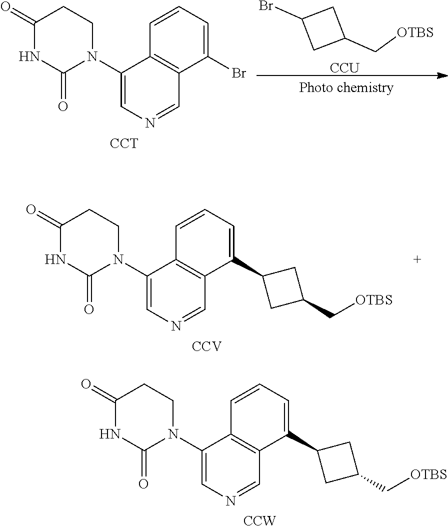

To an 40 mL vial equipped with a stir bar was added 1-(8-bromo-4-isoquinolyl)hexahydropyrimidine-2,4-dione (500 mg, 1.56 mmol, Intermediate CCT), (3-bromocyclobutyl)methoxy-tert-butyl-dimethyl-silane (436 mg, 1.56 mmol, Intermediate CCU), Ir[dF(CF₃)ppy]₂(dtbpy)(PF₆) (17.5 mg, 15.6 umol), NiCl₂.dtbbpy (3.11 mg, 7.81 umol), TTMSS (388 mg, 1.56 mmol), and 2,6-Lutidine (334 mg, 3.12 mmol) in DME (5 mL). The vial was sealed and placed under nitrogen. The reaction was stirred and irradiated with a 50 W [455 nm] blue LED lamp (3 cm away), with cooling water to keep the reaction temperature at 25° C. for 14 hrs. On completion, the reaction mixture was diluted with MeOH (10 mL) and concentrated to give a residue. The residue was purified by column chromatography (SiO², DCM/EA=30:1 to 1:1) to give 1-[8-[3-[[Tert-butyl(dimethyl)silyl]oxymethyl]cyclobutyl]-4-isoquinolyl]hexahydropyrimidine-2,4-dione (150 mg, 19% yield) as a white solid (¹H NMR (400 MHz, DMSO-d₆) δ 10.53 (s, 1H), 9.31 (s, 1H), 8.56 (s, 1H), 7.89-7.81 (m, 1H), 7.81-7.73 (m, 1H), 7.64 (d, J=7.2 Hz, 1H), 4.44 (m, 1H), 3.97-3.83 (m, 3H), 3.71 (d, J=12.0 Hz, 1H), 2.97 (d, J=6.0 Hz, 1H), 2.76 (d, J=5.6 Hz, 1H), 2.42-2.31 (m, 5H), 0.93 (s, 9H), 0.12 (s, 6H); LC-MS (ESI⁺) m/z 440.1 (M+H)⁺), and 1-[8-[3-[[tert-butyl(dimethyl)silyl]oxymethyl]cyclobutyl]-4-isoquinolyl]hexahydropyrimidine-2,4-dione (120 mg, 13% yield) as a white solid (¹H NMR (400 MHz, DMSO-d₆) δ10.51 (s, 1H), 9.90 (s, 1H), 9.40 (s, 1H), 8.55 (s, 1H), 7.83-7.77 (m, 1H), 7.53-7.45 (m, 1H), 4.26-4.07 (m, 1H), 3.99-3.65 (m, 3H), 3.57 (s, 1H), 3.02-2.93 (m, 1H), 2.81-2.71 (m, 1H), 2.45-2.28 (m, 5H), 0.85 (d, J=1.6 Hz, 9H), 0.03 (d, J=1.6 Hz, 6H); LC-MS (ESI+) m/z 440.1 (M+H)⁺).

### 3-[4-(2,4-Dioxohexahydropyrimidin-1-yl)-8-isoquinolyl]cyclobutanecarbaldehyde (Intermediate CCX)

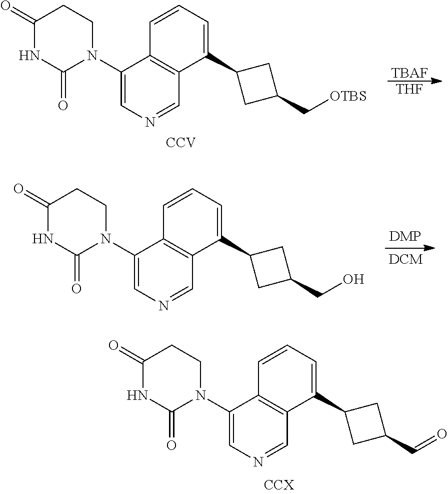

### Step 1—1-[8-[3-(Hydroxymethyl)cyclobutyl]-4-isoquinolyl]hexahydropyrimidine-2,4-dione

To a mixture of 1-[8-[3-[[tert-butyl(dimethyl)silyl]oxymethyl]cyclobutyl]-4-isoquinolyl]hexahydropyrimidine-2,4-dione (140 mg, 318 umol, Intermediate CCV) in THF (2 mL) was added TBAF (1 M, 318 uL). The mixture was stirred at 25° C. for 1 hr. On completion, the mixture was filtered and concentrated to give the title compound (100 mg, 79% yield) as a yellow oil. LC-MS (ESI+) m/z 326.0 (M+H)⁺.

### Step 2—3-[4-(2,4-Dioxohexahydropyrimidin-1-yl)-8-isoquinolyl]cyclobutanecarbaldehyde

To a mixture of 1-[8-[3-(hydroxymethyl)cyclobutyl]-4-isoquinolyl]hexahydropyrimidine-2,4-dione (90.0 mg, 276 umol) in DCM (2 mL) was added DMP (586 mg, 1.38 mmol). The mixture was then stirred at 25° C. for 1 hr. On completion, the reaction mixture was quenched with sat. Na₂S₂O₃ solution (2 mL) and sat. NaHCO₃ solution (2 mL) under stirring. Then the mixture was extracted with DCM (2×5 mL). The combined organic layer was dried over Na₂SO₄, filtered and concentrated in vacuo to give the title compound (50.0 mg, 45% yield) as a white solid. LC-MS (ESI+) m/z 324.0 (M+H)⁺.

### N-[6-methoxy-2-[4-(piperazin-1-ylmethyl)cyclohexyl]indazol-5-yl]-6-(trifluoromethyl) pyridine-2-carboxamide (Intermediate CCY)

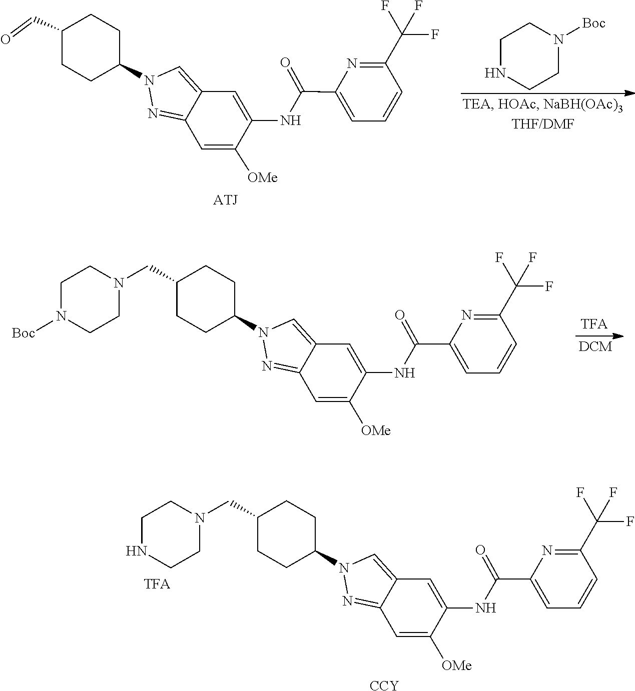 [SMILES: COc1cc2nn([C@H]3CC[C@H](C=O)CC3)cc2cc1NC(=O)c1cccc(C(F)(F)F)n1 ; CC(C)(C)OC(=O)N1CCNCC1 ; COc1cc2nn([C@H]3CC[C@H](CN4CCN(C(=O)OC(C)(C)C)CC4)CC3)cc2cc1NC(=O)c1cccc(C(F)(F)F)n1 ; COc1cc2nn([C@H]3CC[C@H](CN4CCNCC4)CC3)cc2cc1NC(=O)c1cccc(C(F)(F)F)n1 ; "CCY"]

### Step 1—Tert-butyl 4-[[4-[6-methoxy-5-[[6-(trifluoromethyl)pyridine-2-carbonyl]amino]indazol-2-yl] cyclohexyl]methyl]piperazine-1-carboxylate

To a mixture of tert-butyl piperazine-1-carboxylate (450 mg, 2.02 mmol, HCl salt, CAS #143238-38-4) in DMF (3 mL) was added TEA (68.1 mg, 673 umol) at 20° C. until the pH stabilized at 8. The mixture was stirred at 20° C. for 0.25 hr, then AcOH (40.4 mg, 673 umol) was added at 20° C. until the pH stabilized at 5˜6. The mixture was then cooled to −15° C. Subsequently, N-[2-(4-formylcyclohexyl)-6-methoxy-indazol-5-yl]-6-(trifluoromethyl)pyridine-2-carboxamide (300 mg, 673 umol, Intermediate ATJ) was added and the mixture was stirred for 0.5 hr. Next, NaBH(OAc)₃ (285 mg, 1.35 mmol) was added in one portion. The resulting reaction mixture was stirred at −15° C. for 1 hr. On completion, the mixture was filtered and the cake was concentrated in vacuo to give the title compound (240 mg, 52% yield) as a brown solid. ¹H NMR (400 MHz, DMSO-d₆) δ 10.50 (s, 1H), 8.68 (s, 1H), 8.51-8.43 (m, 1H), 8.43-8.36 (m, 1H), 8.32 (s, 1H), 8.21 (dd, J=0.8, 7.6 Hz, 1H), 7.15 (s, 1H), 4.42-4.27 (m, 1H), 3.98 (s, 3H), 3.29-3.16 (m, 4H), 2.64-2.53 (m, 1H), 2.29 (t, J=4.8 Hz, 3H), 2.19-2.09 (m, 4H), 1.98-1.81 (m, 4H), 1.69-1.52 (m, 1H), 1.45-1.34 (m, 9H), 1.20-1.01 (m, 2H); LC-MS (ESI+) m/z 617.2 (M+H)⁺.

### Step 2—N-[6-methoxy-2-[4-(piperazin-1-ylmethyl)cyclohexyl]indazol-5-yl]-6-(trifluoromethyl) pyridine-2-carboxamide

To a mixture of tert-butyl 4-[[4-[6-methoxy-5-[[6-(trifluoromethyl)pyridine-2-carbonyl]amino]indazol-2-yl]cyclohexyl]methyl]piperazine-1-carboxylate (40.0 mg, 64.8 umol) in DCM (1 mL) was added TFA (0.5 mL). The mixture was stirred at 25° C. for 1 hr. On completion, the mixture was filtered and concentrated to give a residue to give the title compound (40.9 mg, 99% yield, TFA salt) as a brown solid. LC-MS (ESI⁺) m/z 517.2 (M+H)⁺.

### 3-[4-(2,4-Dioxohexahydropyrimidin-1-yl)-8-isoquinolyl]cyclobutanecarbaldehyde (Intermediate CCZ)

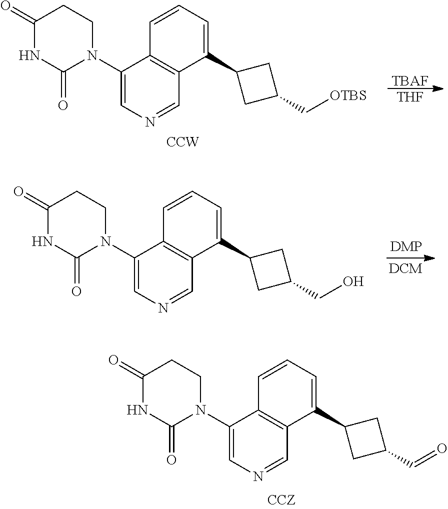

### Step 1—1-[8-[3-(hydroxymethyl)cyclobutyl]-4-isoquinolyl]hexahydropyrimidine-2,4-dione

To a solution of 1-[8-[3-[[tert-butyl(dimethyl)silyl]oxymethyl]cyclobutyl]-4-isoquinolyl]hexahydro-pyrimidine-2,4-dione (80 mg, 181.9 umol, Intermediate CCW) in DCM (1 mL) was added HCl/dioxane (4 M, 1.60 mL). The mixture was stirred at 25° C. for 0.5 hr. On completion, the mixture was concentrated to give the title compound (59.2 mg, 164 umol, 90% yield) as a white solid. LC-MS (ESI+) m/z 326.0 (M+H)⁺.

### Step 2—3-[4-(2,4-Dioxohexahydropyrimidin-1-yl)-8-isoquinolyl]cyclobutanecarbaldehyde

To a solution of 1-[8-[3-(hydroxymethyl)cyclobutyl]-4-isoquinolyl]hexahydropyrimidine-2,4-dione (59.2 mg, 182 umol) in DCM (5 mL) and DMSO (0.1 mL) was added DMP (154.3 mg, 364 umol). The mixture was stirred at 25° C. for 1 hr. On completion, the reaction mixture was quenched by addition of Na₂S₂O₄ (5 mL) and NaHCO₃ (5 mL). The reaction mixture was filtered and concentrated in vacuo. The residue was diluted with DCM (20 mL). The mixture was filtered and concentrated to give the title compound (56 mg, 67% yield) as a white solid. LC-MS (ESI+) m/z 324 (M+H)⁺.

### N-[2-(4-Formylcyclohexyl)-6-methoxy-indazol-5-yl]-5-methoxy-pyridine-3-carboxamide (Intermediate CEE)

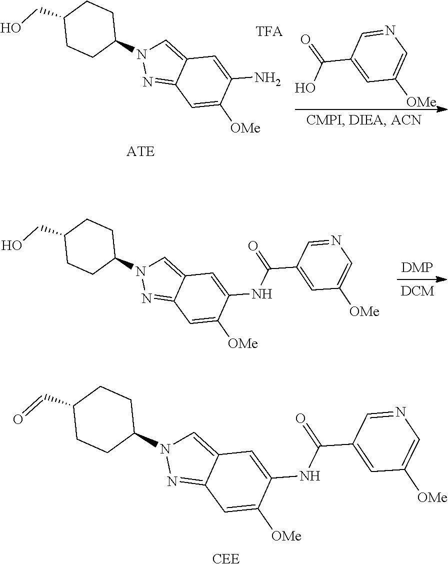 [SMILES: COc1cc2nn([C@H]3CC[C@H](CO)CC3)cc2cc1N ; COc1cncc(C(=O)O)c1 ; COc1cncc(C(=O)Nc2cc3cn([C@H]4CC[C@H](CO)CC4)nc3cc2OC)c1 ; COc1cncc(C(=O)Nc2cc3cn([C@H]4CC[C@H](C=O)CC4)nc3cc2OC)c1]

### Step 1—N-[2-[4-(hydroxymethyl)cyclohexyl]-6-methoxy-indazol-5-yl]-5-methoxy-pyridine-3-carboxamide

A mixture of [4-(5-amino-6-methoxy-indazol-2-yl)cyclohexyl]methanol (518 mg, 1.33 mmol, TFA, Intermediate ATE) and DIEA (515 mg, 3.99 mmol) in ACN (5 mL) was stirred for 2 min. Then a mixture of 5-methoxypyridine-3-carboxylic acid (203 mg, 1.33 mmol, CAS #1044919-31-4), DIEA (515 mg, 3.99 mmol) and CMPI (407 mg, 1.60 mmol) in ACN (2 mL) was added. The reaction mixture was stirred at 25° C. for 1 hr. On completion, the residue was diluted with water (20 mL), then the residue was extracted with EA (3×30 mL). The combined organic layer was dried over Na₂SO₄, filtered and concentrated in vacuo to give a residue. Then the residue was diluted with EA (10 ml) and the NaHCO₃ (100 mg, solid) was added at 25° C. for 10 min. Then the residue was extracted with EA (3×30 mL), dried over Na₂SO₄, filtered and concentrated to give title compound (310 mg, 56% yield) as yellow solid. ¹H NMR (400 MHz, DMSO-d₆) δ 9.65 (s, 1H), 8.72 (s, 1H), 8.47 (d, J=2.8 Hz, 1H), 8.29 (s, 1H), 8.01 (s, 1H), 7.85 (s, 1H), 7.07 (s, 1H), 4.48 (t, J=5.2 Hz, 1H), 4.39-4.32 (m, 1H), 3.91 (s, 3H), 3.86 (s, 3H), 3.29 (t, J=5.6 Hz, 2H), 2.17-2.11 (m, 2H), 1.93-1.86 (m, 4H), 1.53-1.42 (m, 1H), 1.20-1.09 (m, 2H); LC-MS (ESI⁺) m/z 411.2 (M+H)⁺.

### Step 2—N-[2-(4-Formylcyclohexyl)-6-methoxy-indazol-5-yl]-5-methoxy-pyridine-3-carboxamide

To a mixture of N-[2-[4-(hydroxymethyl)cyclohexyl]-6-methoxy-indazol-5-yl]-5-methoxy-pyridine-3-carboxamide (310 mg, 755 umol) in DCM (5 mL) was added DMP (480 mg, 1.13 mmol). The reaction mixture was stirred at 25° C. for 1 hr. On completion, the reaction mixture was quenched by saturated Na₂S₂O₃ (8 mL) and saturated NaHCO₃ (8 mL) at 25° C., and then stirred for 30 minutes. The mixture was extracted with DCM (2×20 mL), then the combined organic layer was dried over Na₂SO₄, filtered and concentrated in vacuo to give title compound (300 mg, 97% yield) as red solid. ¹H NMR (400 MHz, DMSO-d₆) δ 9.65 (d, J=7.6 Hz, 2H), 8.72 (s, 1H), 8.47 (d, J=2.8 Hz, 1H), 8.30 (s, 1H), 7.85 (s, 1H), 7.80-7.74 (m, 1H), 7.07 (s, 1H), 4.44-4.36 (m, 1H), 3.91 (s, 3H), 3.86 (s, 3H), 2.45-2.38 (m, 1H), 2.22-22.18 (m, 2H), 2.13-2.09 (m, 2H), 2.01-1.93 (m, 2H), 1.49-1.39 (m, 2H). LC-MS (ESI⁺) m/z 409.2 (M+H)⁺.

### Tert-butyl (S)-3-(4-(3-(4-methoxybenzyl)-2,4-dioxotetrahydropyrimidin-1(2H)-yl)isoquinolin-8-yl)pyrrolidine-1-carboxylate (Intermediate CEF) and tert-butyl (R)-3-(4-(3-(4-methoxybenzyl)-2,4-dioxotetrahydropyrimidin-1(2H)-yl)isoquinolin-8-yl)pyrrolidine-1-carboxylate (Intermediate CEG)

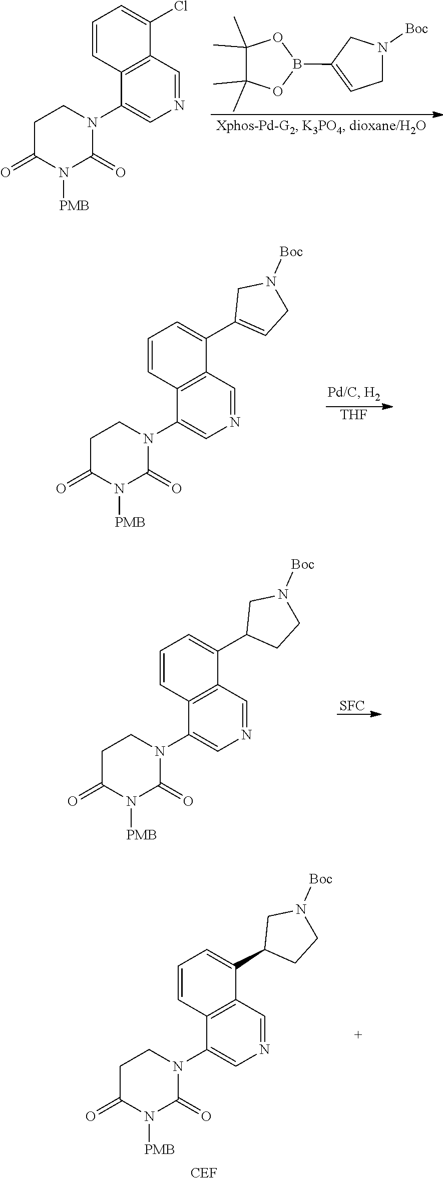

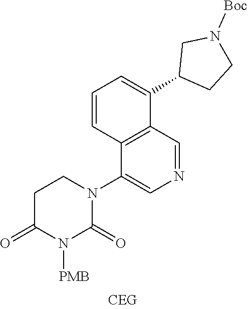

### Step 1—Tert-butyl3-[4-[3-[(4-methoxyphenyl)methyl]-2,4-dioxo-hexahydropyrimidin-1-yl]-8-isoquinolyl]-2,5-dihydropyrrole-1-carboxylate

To a solution of tert-butyl 3-(4,4,5,5-tetramethyl-1,3,2-dioxaborolan-2-yl)-2,5-dihydropyrrole-1-carboxylate (2.91 g, 9.85 mmol, CAS #212127-83-8) and 1-(8-chloro-4-isoquinolyl)-3-[(4-methoxyphenyl)methyl]hexahydropyrimidine-2,4-dione (3 g, 7.58 mmol, synthesized via Steps 1-2 of Intermediate BSL) in dioxane (30 mL) and H₂O (10 mL) was added K₃PO₄ (4.83 g, 22.7 mmol) and XPHOS-PD-G2 (596 mg, 757 umol). Then the mixture was stirred at 80° C. for 5 hrs. On completion, the reaction mixture was filtered and concentrated in vacuo to give a residue. The residue was purified by reverse phase (0.1% FA condition) to give the title compound (2 g, 49% yield) as a white solid. ¹H NMR (400 MHz, DMSO-d₆) δ 9.54 (s, 1H), 8.60 (s, 1H), 7.91 (d, J=8.4 Hz, 1H), 7.79 (t, J=7.6 Hz, 1H), 7.71 (d, J=6.8 Hz, 1H), 7.26 (d, J=8.4 Hz, 2H), 6.88 (d, J=8.4 Hz, 2H), 6.21 (d, J=11.6 Hz, 1H), 5.75 (s, 1H), 4.84 (s, 2H), 4.55 (s, 2H), 4.38 (d, J=3.6 Hz, 2H), 4.05-3.87 (m, 1H), 3.73 (s, 3H), 3.21-3.11 (m, 1H), 2.97 (d, J=5.6, 16.4 Hz, 1H), 1.46 (d, J=7.9 Hz, 9H). LC-MS (ESI⁺) m/z 529.3 (M+H)⁺.

### Step 2—Tert-butyl 3-[4-[3-[(4-methoxyphenyl)methyl]-2,4-dioxo-hexahydropyrimidin-1-yl]-8-isoquinolyl]pyrrolidine-1-carboxylate

To a solution of tert-butyl 3-[4-[3-[(4-methoxyphenyl)methyl]-2,4-dioxo-hexahydropyrimidin-1-yl]-8-isoquinolyl]-2,5-dihydropyrrole-1-carboxylate (1 g, 1.89 mmol) in THF (10 mL) was added Pd/C (1 g, 10 wt %), then the mixture was stirred at 25° C. for 2 hrs under H₂ (15 psi). On completion, the reaction mixture was filtered and concentrated in vacuo to give a residue. The residue was purified by column chromatography (SiO₂, PE:EA=50:1 to PE:EA=3:1) to give the title compound (800 mg, 79% yield) as a yellow solid. ¹H NMR (400 MHz, DMSO-d₆) δ 9.68 (s, 1H), 8.61 (s, 1H), 7.88-7.73 (m, 2H), 7.66 (d, J=6.4 Hz, 1H), 7.27 (d, J=8.8 Hz, 2H), 6.93-6.86 (m, 2H), 4.84 (s, 2H), 4.43-4.30 (m, 1H), 3.99-3.77 (m, 3H), 3.74 (s, 3H), 3.55-3.41 (m, 3H), 3.20-3.11 (m, 1H), 2.98 (d, J=5.6, 16.4 Hz, 1H), 2.42-2.32 (m, 1H), 2.19-2.07 (m, 1H), 1.43 (d, J=9.6 Hz, 9H). LC-MS (ESI⁺) m/z 531.4 (M+H)⁺.

### Step 3—Tert-butyl (S)-3-(4-(3-(4-methoxybenzyl)-2,4-dioxotetrahydropyrimidin-1(2H)-yl)isoquinolin-8-yl)pyrrolidine-1-carboxylate and tert-butyl (R)-3-(4-(3-(4-methoxybenzyl)-2,4-dioxotetrahydropyrimidin-1(2H)-yl)isoquinolin-8-yl)pyrrolidine-1-carboxylate

Tert-butyl 3-[4-(2,4-dioxohexahydropyrimidin-1-yl)-8-isoquinolyl]pyrrolidine-1-carboxylate (380 mg, 925 umol) separated by SFC to give tert-butyl (S)-3-(4-(3-(4-methoxybenzyl)-2,4-dioxotetrahydropyrimidin-1(2H)-yl)isoquinolin-8-yl)pyrrolidine-1-carboxylate (190 mg, 50% yield, tR=1.48) as a white solid (¹H NMR (400 MHz, DMSO-d₆) δ 10.54 (s, 1H), 9.66 (s, 1H), 8.59 (s, 1H), 7.90 (d, J=8.4 Hz, 1H), 7.79 (t, J=7.6 Hz, 1H), 7.65 (d, J=6.8 Hz, 1H), 4.49-4.36 (m, 1H), 3.97-3.80 (m, 2H), 3.76-3.66 (m, 1H), 3.53-3.36 (m, 3H), 2.98 (d, J=6.4, 16.4 Hz, 1H), 2.76-2.74 (m, 1H), 2.42-2.35 (m, 1H), 2.19-2.07 (m, 1H), 1.43 (d, J=9.6 Hz, 9H). LC-MS (ESI⁺) m/z 411.3 (M−120+H)⁺) and tert-butyl (R)-3-(4-(3-(4-methoxybenzyl)-2,4-dioxotetrahydropyrimidin-1(2H)-yl)isoquinolin-8-yl)pyrrolidine-1-carboxylate (80 mg, 42% yield, tR=1.61). ¹H NMR (400 MHz, DMSO-d₆) δ 9.66 (s, 1H), 8.59 (s, 1H), 7.90 (d, J=8.4 Hz, 2H), 4.50-4.33 (m, 1H), 4.02-3.80 (m, 2H), 3.76-3.64 (m, 1H), 3.54-3.35 (m, 3H), 2.98 (d, J=6.0, 10.0, 16.4 Hz, 1H), 2.82-2.65 (m, 2H), 2.21-2.07 (m, 2H), 1.42 (d, J=9.6 Hz, 9H); LC-MS (ESI⁺) m/z 411.3 (M−120+H)⁺. Absolute stereochemistry was arbitrarily assigned.

### 1-[8-[(3S)-pyrrolidin-3-yl]-4-isoquinolyl]hexahydropyrimidine-2,4-dione (Intermediate CEH)

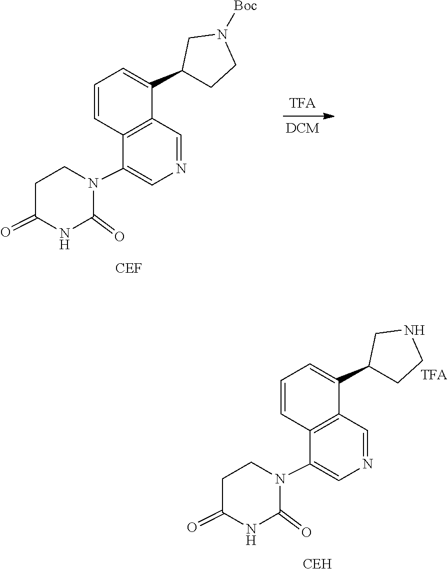 [SMILES: CC(C)(C)OC(=O)N1CC[C@@H](c2cccc3c(N4CCC(=O)NC4=O)cncc23)C1 ; O=C1CCN(c2cncc3c([C@@H]4CCNC4)cccc23)C(=O)N1]

To a solution of tert-butyl (3S)-3-[4-(2,4-dioxohexahydropyrimidin-1-yl)-8-isoquinolyl]pyrrolidine-1-carboxylate (30 mg, 73.0 umol) in DCM (0.5 mL) was added TFA (462 mg, 4.05 mmol), then the mixture was stirred at 25° C. for 1 hr. On completion, the reaction mixture was concentrated in vacuo to give the title compound (30 mg, 96% yield) as a yellow oil. LC-MS (ESI⁺) m/z 311.3 (M+H)⁺.

### N-[2-[4-(iodomethyl)cyclohexyl]-6-methoxy-indazol-5-yl]-6-(trifluoromethyl)pyridine-2-carboxamide (Intermediate CEI)

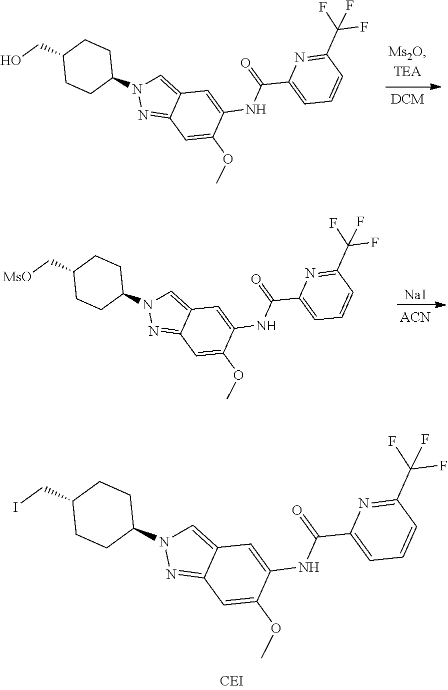 [SMILES: COc1cc2nn([C@H]3CC[C@H](CO)CC3)cc2cc1NC(=O)c1cccc(C(F)(F)F)n1 ; COc1cc2nn([C@H]3CC[C@H](COS(C)(=O)=O)CC3)cc2cc1NC(=O)c1cccc(C(F)(F)F)n1 ; "ACN" ; "NaI" ; COc1cc2nn([C@H]3CC[C@H](CI)CC3)cc2cc1NC(=O)c1cccc(C(F)(F)F)n1]

### Step 1—[4-[6-Methoxy-5-[[6-(trifluoromethyl)pyridine-2-carbonyl]amino]indazol-2-yl]cyclohexyl]methyl methanesulfonate

To a solution of N-[2-[4-(hydroxymethyl)cyclohexyl]-6-methoxy-indazol-5-yl]-6-(trifluoromethyl) pyridine-2-carboxamide (300 mg, 668 umol, synthesized via Step 1 of Intermediate ATJ) in DCM (3 mL) was added TEA (203 mg, 2.01 mmol) and methylsulfonyl methanesulfonate (174 mg, 1.00 mmol). The reaction mixture was stirred at 25° C. for 3 hrs. On completion, the reaction mixture was diluted with H₂O (10 mL), and extracted with DCM (2×15 mL). The combined organic layer was washed with brine (2×15 mL), dried over Na₂SO₄ and filtered. The filtrate was concentrated in vacuo to give the title compound (320 mg, 90% yield) as a white solid. LC-MS (ESI⁺) m/z 527.1 (M+H)⁺.

### Step 2—N-[2-[4-(iodomethyl)cyclohexyl]-6-methoxy-indazol-5-yl]-6-(trifluoromethyl)pyridine-2-carboxamide

To a solution of [4-[6-methoxy-5-[[6-(trifluoromethyl) pyridine-2-carbonyl]amino]indazol-2-yl]cyclohexyl]methyl methanesulfonate (320 mg, 607 umol) in ACN (4 mL) was added NaI (455 mg, 3.04 mmol). The reaction was stirred at 80° C. for 16 hrs. On completion, the reaction mixture was diluted with H₂O (15 mL), and extracted with EA (2×20 mL). The combined organic layer was washed with brine (2×20 mL), dried over Na₂SO₄ and filtered. The filtrate was concentrated in vacuo to give a residue. The residue was purified by reverse phase (0.1% FA condition) to give the title compound (240 mg, 70% yield) as a yellow solid. ¹H NMR (400 MHz, DMSO-d₆) δ 10.48 (s, 1H), 8.67 (s, 1H), 8.47-8.42 (m, 1H), 8.41-8.36 (m, 1H), 8.30 (s, 1H), 8.19 (d, J=7.6 Hz, 1H), 7.14 (s, 1H), 4.43-4.27 (m, 1H), 3.97 (s, 3H), 3.28 (d, J=6.0 Hz, 2H), 2.13 (d, J=11.6 Hz, 2H), 2.04-1.85 (m, 4H), 1.58-1.45 (m, 1H), 1.31-1.16 (m, 2H); LC-MS (ESI+) m/z 559.0 (M+H)⁺.

### Tert-butyl (4R)-2,2-dimethyl-4-[3-methyl-2-oxo-1-(2-trimethylsilylethoxymethyl) benzimidazol-4-yl]piperidine-1-carboxylate (Intermediate CEJ) and tert-butyl (4S)-2,2-dimethyl-4-[3-methyl-2-oxo-1-(2-trimethylsilylethoxymethyl)benzimidazol-4-yl]piperidine-1-carboxylate (Intermediate CEK)

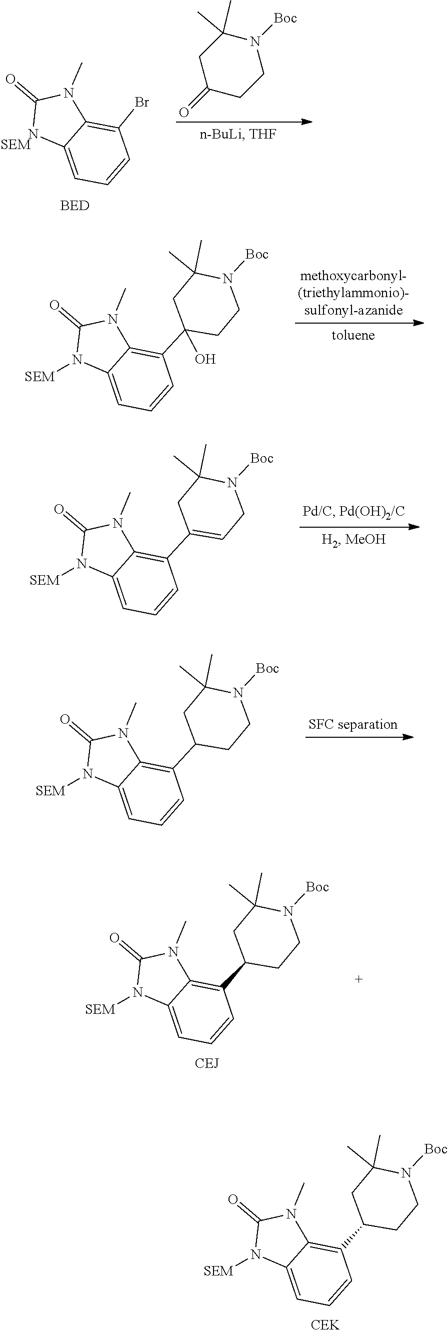

### Step 1—Tert-butyl 4-hydroxy-2,2-dimethyl-4-[3-methyl-2-oxo-1-(2-trimethylsilylethoxymethyl) benzimidazol-4-yl]piperidine-1-carboxylate

To a solution of 4-bromo-3-methyl-1-(2-trimethylsilylethoxymethyl)benzimidazol-2-one (714 mg, 2.00 mmol, Intermediate BED) in THF(10 mL) was added n-BuLi (2.5 M, 879 uL) at −78° C., the reaction mixture was stirred at −78° C. for 30 mins, then tert-butyl 2,2-dimethyl-4-oxo-piperidine-1-carboxylate (500 mg, 2.20 mmol, CAS #346893-03-1) was added to the mixture. Then the reaction mixture was stirred at −78° C. for 1 hr. On completion, the reaction mixture was quenched with NH₄Cl (10 mL), then extracted with EA (2×20 mL). The organic phase was combined and dried with Na₂SO₄, then filtered and the filtrate was concentrated in vacuo to give a residue. The residue was purified by reverse phase (0.1% FA condition) to give the title compound (1.50 g, 37% yield) as a white solid. LC-MS (ESI⁺) m/z 506.1 (M+H)⁺.

### Step 2—Tert-butyl6,6-dimethyl-4-[3-methyl-2-oxo-1-(2-trimethylsilylethoxymethyl)benzimidazol-4-yl]-2,5-dihydropyridine-1-carboxylate

To a solution of tert-butyl 4-hydroxy-2,2-dimethyl-4-[3-methyl-2-oxo-1-(2-trimethylsilylethoxymethyl)benzimidazol-4-yl]piperidine-1-carboxylate (1.50 g, 2.97 mmol) in toluene (5 mL) was added methoxycarbonyl-(triethylammonio)sulfonylazanide (1.06 g, 4.45 mmol). Then the reaction mixture was stirred at 90° C. for 12 hrs. On completion, the reaction mixture was concentrated in vacuo to give a residue. The residue was purified by reverse phase (0.1% FA condition) to give the title compound (1.00 g, 69% yield) as colorless oil. ¹H NMR (400 MHz, DMSO-d₆) δ 7.17-7.12 (m, 1H), 7.08-7.04 (m, 1H), 6.86-6.84 (m, 1H), 5.86 (t, J=4.0 Hz, 1H), 5.25 (s, 2H), 4.03-3.94 (m, 2H), 3.55 (t, J=8.0 Hz, 2H), 3.30 (s, 3H), 2.46 (s, 2H), 1.45 (s, 6H), 1.43 (s, 9H), 0.87-0.83 (m, 2H), 0.06 (s, 9H).

### Step 3—Tert-butyl 2,2-dimethyl-4-[3-methyl-2-oxo-1-(2-trimethylsilylethoxymethyl)benzimidazol-4-yl]piperidine-1-carboxylate

To a solution of tert-butyl 6,6-dimethyl-4-[3-methyl-2-oxo-1-(2-trimethylsilylethoxymethyl) benzimidazol-4-yl]-2,5-dihydropyridine-1-carboxylate (1.00 g, 2.05 mmol) in MeOH (20 mL) was added Pd/C (0.50 g, 10 wt %). The reaction mixture was then stirred at 40° C. for 16 hrs under H₂ (50 psi). On completion, the reaction mixture was filtered and the filtrate was concentrated in vacuo to give the title compound (980 mg, 98% yield) as a white solid. ¹H NMR (400 MHz, DMSO-d₆) δ 7.11-6.99 (m, 3H), 5.46 (s, 1H), 5.23 (s, 2H), 3.91-3.85 (m, 1H), 3.61 (s, 3H), 3.55-3.50 (m, 2H), 3.17 (d, J=5.2 Hz, 2H), 1.89-1.86 (m, 1H), 1.77-1.70 (m, 2H), 1.50-1.35 (m, 15H), 0.88-0.79 (m, 2H), 0.06 (m, 9H).

### Step 4—Tert-butyl (4R)-2,2-dimethyl-4-[3-methyl-2-oxo-1-(2-trimethylsilylethoxymethyl) benzimidazol-4-yl]piperidine-1-Carboxylate and tert-butyl (4S)-2,2-dimethyl-4-[3-methyl-2-oxo-1-(2-trimethylsilylethoxymethyl)benzimidazol-4-yl]piperidine-1-carboxylate

Tert-butyl 2, 2-dimethyl-4-[3-methyl-2-oxo-1-(2-trimethylsilylethoxymethyl)benzimidazol-4-yl]piperidine-1-carboxylate (1.00 g, 2.04 mmol) separated by SFC (column: DAICEL CHIRALPAK IC(250 mm*30 mm, 10 um); mobile phase: [0.1% NH₃·H₂O MEOH]; B %: 40%-40%, 7; 150 min) and SFC(column: Phenomenex-Cellulose-2 (250 mm*30 mm, 10 um); mobile phase: [0.1% NH₃·H₂O MEOH]; B %: 20%-20%, 2.1; 30 min) to give tert-butyl (4R)-2,2-dimethyl-4-[3-methyl-2-oxo-1-(2-trimethylsilylethoxymethyl) benzimidazol-4-yl]piperidine-1-carboxylate (240 mg, 24% yield) as a white solid (¹H NMR (400 MHz, DMSO-d₆) δ 7.13-7.09 (m, 3H), 5.29 (s, 2H), 3.96-3.93 (m, 1H), 3.68 (s, 3H), 3.65-3.56 (m, 3H), 3.32-3.22 (m, 1H), 2.00-1.90 (m, 1H), 1.85-1.72 (m, 3H), 1.56 (s, 3H), 1.48 (s, 9H), 1.43 (s, 3H), 0.94-0.85 (m, 2H), 0.008 (s, 9H), LC-MS (ESI⁺) m/z 490.2 (M+H)⁺) and tert-butyl (4S)-2,2-dimethyl-4-[3-methyl-2-oxo-1-(2-trimethylsilylethoxymethyl)benzimidazol-4-yl]piperidine-1-carboxylate (322 mg, 32% yield) as a white solid (¹H NMR (400 MHz, DMSO-d₆) δ 7.10-7.00 (m, 3H), 5.23 (s, 2H), 3.90-3.60 (m, 1H), 3.61 (s, 3H), 3.60-3.50 (m, 3H), 3.22-3.20 (m, 1H), 1.95-1.84 (m, 1H), 1.78-1.65 (m, 3H), 1.49 (s, 3H), 1.42 (s, 9H), 1.37 (s, 3H), 0.89-0.80 (m, 2H), 0.07 (s, 9H), LC-MS (ESI⁺) m/z 490.2 (M+H)⁺). Absolute stereochemistry was assigned arbitrarily.

### 3-[4-[(4S)-2,2-dimethyl-4-piperidyl]-3-methyl-2-oxo-benzimidazol-1-yl]piperidine-2,6-dione (Intermediate CEL)

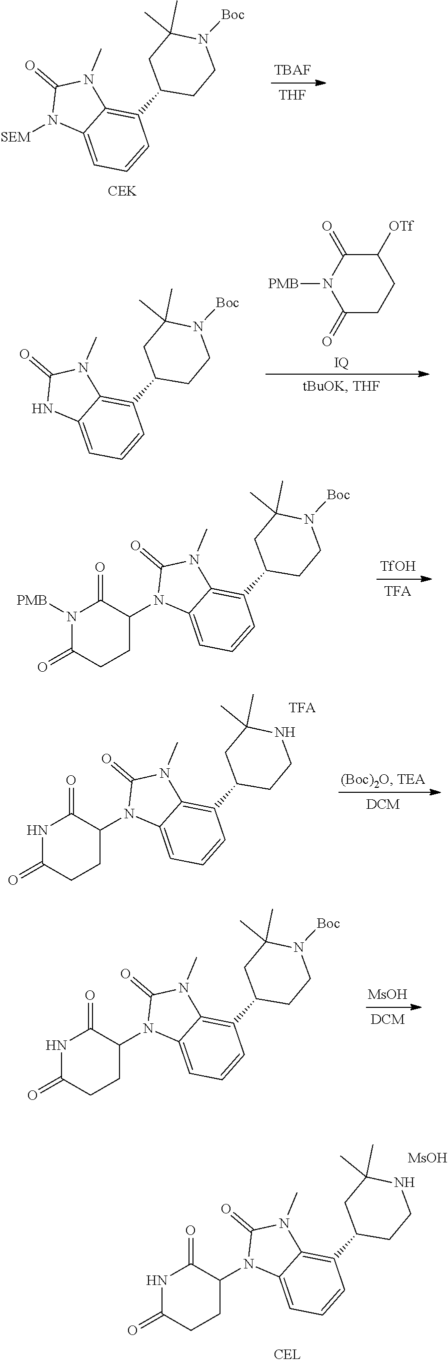

### Step 1—Tert-butyl (4S)-2,2-dimethyl-4-(3-methyl-2-oxo-1H-benzimidazol-4-yl)piperidine-1-carboxylate

To a solution of tert-butyl (4S)-2, 2-dimethyl-4-[3-methyl-2-oxo-1-(2-trimethylsilylethoxymethyl) benzimidazol-4-yl]piperidine-1-carboxylate (300 mg, 612 umol, Intermediate CEK) in THF (3 mL) was added TBAF (1 M, 3.06 mL). The reaction mixture was stirred at 70° C. for 16 hrs. On completion, the reaction mixture was diluted with H₂O (15 mL), and extracted with EA (2×20 mL). The combined organic layer was washed with brine (2×20 mL), dried over Na₂SO₄ and filtered. The filtrate was concentrated in vacuo to give the title compound (210 mg, 95% yield) as a white solid. LC-MS (ESI+) m/z 304.2 (M+H−56)+.

### Step 2—Tert-butyl (4S)-4-[1-[1-[(4-methoxyphenyl) methyl]-2,6-dioxo-3-piperidyl]-3-methyl-2-oxo-benzimidazol-4-yl]-2,2-dimethyl-piperidine-1-carboxylate

To a solution of tert-butyl (4S)-2, 2-dimethyl-4-(3-methyl-2-oxo-1H-benzimidazol-4-yl) piperidine-1-carboxylate (135 mg, 375 umol) in THF (2 mL) was added t-BuOK (63.2 mg, 563 umol). The mixture was stirred at −10° C. for 30 mins. Then a solution of [1-[(4-methoxyphenyl) methyl]-2, 6-dioxo-3-piperidyl]trifluoromethanesulfonate (214 mg, 563 umol, Intermediate IQ) in THF (2 mL) was added dropwise to the mixture, and the reaction mixture was stirred at −10° C. for 30 mins. On completion, the reaction mixture was diluted with H₂O (15 mL), and extracted with EA (2×20 mL). The combined organic layer was washed with brine (2×20 mL), dried over Na₂SO₄ and filtered. The filtrate was concentrated in vacuo to give a residue. The residue was purified by reverse phase (0.1% FA condition) to give the title compound (125 mg, 56% yield) as a white solid. ¹H NMR (400 MHz, DMSO-d₆) δ 7.21 (d, J=8.4 Hz, 2H), 7.06-6.93 (m, 2H), 6.85 (d, J=8.4 Hz, 3H), 5.52 (dd, J=5.2, 12.8 Hz, 1H), 4.90-4.68 (m, 2H), 3.89 (td, J=4.4, 13.2 Hz, 1H), 3.75-3.67 (m, 3H), 3.62 (s, 3H), 3.59-3.53 (m, 1H), 3.23-3.17 (m, 1H), 3.10-2.99 (m, 1H), 2.86-2.69 (m, 2H), 2.06-2.01 (m, 1H), 1.90 (td, J=4.4, 8.8 Hz, 1H), 1.80-1.65 (m, 3H), 1.49 (s, 3H), 1.42 (s, 9H), 1.37 (s, 3H); LC-MS (ESI+) m/z 591.5 (M+H)⁺.

### Step 3-3-[4-[(4S)-2, 2-Dimethyl-4-piperidyl]-3-methyl-2-oxo-benzimidazol-1-yl]piperidine-2,6-dione

To a solution of tert-butyl (4S)-4-[1-[1-[(4-methoxyphenyl)methyl]-2,6-dioxo-3-piperidyl]-3-methyl-2-oxo-benzimidazol-4-yl]-2,2-dimethyl-piperidine-1-carboxylate (120 mg, 203.15 umol) and in TFA (1.5 mL) was added TfOH (510 mg, 3.40 mmol). Then the reaction mixture was stirred at 70° C. for 2 hrs. On completion, the reaction mixture was concentrated in vacuo to give the title compound (90.0 mg, 91% yield, TFA) as a yellow solid. LC-MS (ESI+) m/z 371.1 (M+H)⁺.

### Step 4—Tert-butyl (4S)-4-[1-(2,6-dioxo-3-piperidyl)-3-methyl-2-oxo-benzimidazol-4-yl]-2,2-dimethyl-piperidine-1-carboxylate

To a solution of 3-[4-[(4S)-2, 2-dimethyl-4-piperidyl]-3-methyl-2-oxo-benzimidazol-1-yl]piperidine-2,6-dione (90.0 mg, 185 umol, TFA) in DCM (1 mL) was added TEA (93.9 mg, 928 umol). Then Boc₂O (48.6 mg, 222 umol) was added. The reaction was stirred at 25° C. for 16 hrs. On completion, the reaction mixture was diluted with H₂O (5 mL) and extracted with DCM (2×5 mL). The combined organic layer was washed with brine (2×5 mL), dried over Na₂SO₄ and filtered. The filtrate was concentrated in vacuo to give a residue. The residue was purified by reverse phase (neutral condition) to give the title compound (70.0 mg, 80% yield) as a white solid. ¹H NMR (400 MHz, DMSO-d₆) δ 11.08 (s, 1H), 7.07-6.93 (m, 3H), 5.37 (dd, J=5.2, 12.4 Hz, 1H), 3.89 (td, J=4.4, 13.6 Hz, 1H), 3.62 (s, 3H), 3.27-3.17 (m, 1H), 2.95-2.82 (m, 1H), 2.78-2.55 (m, 3H), 2.05-1.95 (m, 1H), 1.90 (dd, J=3.2, 12.4 Hz, 1H), 1.83-1.64 (m, 3H), 1.49 (s, 3H), 1.42 (s, 9H), 1.37 (s, 3H); LC-MS (ESI+) m/z 471.2 (M+H)⁺.

### Step 5—3-[4-[(4S)-2, 2-dimethyl-4-piperidyl]-3-methyl-2-oxo-benzimidazol-1-yl]piperidine-2,6-dione

To a solution of tert-butyl (4S)-4-[1-(2, 6-dioxo-3-piperidyl)-3-methyl-2-oxo-benzimidazol-4-yl]-2,2-dimethyl-piperidine-1-carboxylate (70.0 mg, 148 umol) in DCM (0.5 mL) was added MsOH (42.8 mg, 446 umol). The reaction was stirred at 25° C. for 1 hr. On completion, the reaction mixture was concentrated in vacuo to give the title compound (69.0 mg, 99% yield, MsOH) as a colorless oil. LC-MS (ESI+) m/z 371.1 (M+H)⁺.

### N-[6-ethoxy-2-(4-formylcyclohexyl)indazol-5-yl]-6-(trifluoromethyl)pyridine-2-carboxamide (Intermediate CEM)

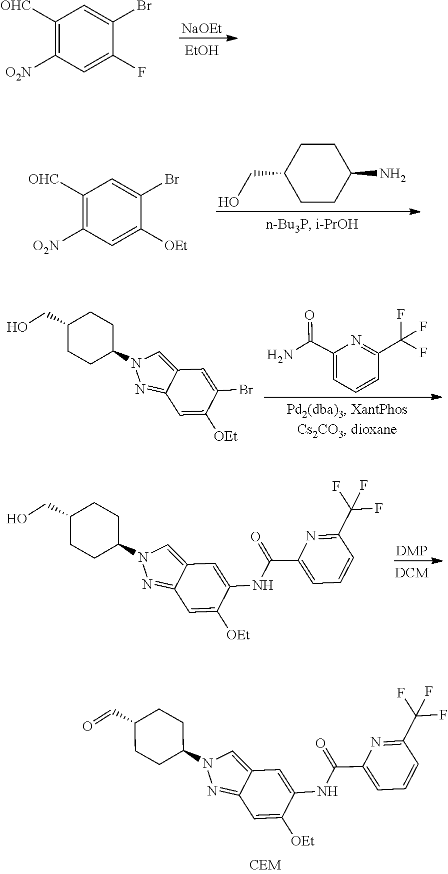

### Step 1—5-Bromo-4-ethoxy-2-nitro-benzaldehyde

The solution of 5-bromo-4-fluoro-2-nitro-benzaldehyde (3.0 g, 12.1 mmol, synthesized via Step 1 of Intermediate ATE) in EtOH (60 mL) was cooled to 0° C., then NaOEt (988 mg, 14.5 mmol) was added. The reaction mixture was then stirred at 25° C. for 12 hrs. On completion, the reaction mixture was quenched with water (50 mL), then extracted by EtOAc (100 ml×3). The organic layers were combined, washed with brine (50 mL), dried over anhydrous Na₂SO₄, and concentrated in vacuo to give the residue. The residue was purified by column chromatography (SiO₂, Petroleum ether/Ethyl acetate=50/1 to 30/1) to give the title compound (770 mg, 23% yield) as yellow solid. ¹H NMR (400 MHz, DMSO-d₆) δ 10.03 (s, 1H), 8.16 (s, 1H), 7.77 (s, 1H), 4.34-4.32 (m, 2H), 1.41 (t, J=6.8 Hz, 3H).

### Step 2—[4-(5-Bromo-6-ethoxy-indazol-2-yl)cyclohexyl]methanol

A solution of 5-bromo-4-ethoxy-2-nitro-benzaldehyde (1.19 g, 4.34 mmol) and (4-aminocyclohexyl) methanol (617 mg, 4.78 mmol, CAS #1467-84-1) in IPA (30 mL) was stirred at 80° C. for 12 hrs under N₂ atmosphere. Then the mixture was cooled to 25° C., and tributylphosphane (2.64 g, 13.0 mmol) was added. The reaction mixture was stirred at 80° C. for 6 hrs under N₂ atmosphere. On completion, the reaction mixture was concentrated in vacuo to give the residue. The residue was purified by column chromatography (SiO₂, Petroleum ether/Ethyl acetate=30/1 to 1/1) to give the title compound (740 mg, 46% yield) as orange solid. ¹H NMR (400 MHz, DMSO-d₆) δ 8.26 (s, 1H), 7.95 (s, 1H), 7.08 (s, 1H), 4.54-4.52 (m, 1H), 4.49 (t, J=5.2 Hz, 1H), 4.36-4.34 (m, 1H), 4.09-4.06 (m, 2H), 3.28 (t, J=6.0 Hz, 2H), 2.17-2.05 (m, 2H), 1.94-1.82 (m, 3H), 1.39 (t, J=6.8 Hz, 3H), 1.15 (s, 2H), 0.91-0.83 (m, 1H). LC-MS (ESI+) m/z 353.1 (M+H)⁺.

### Step 3—N-[6-ethoxy-2-[4-(hydroxymethyl)cyclohexyl]indazol-5-yl]-6-(trifluoromethyl)pyridine-2-carboxamide

A mixture of [4-(5-bromo-6-ethoxy-indazol-2-yl)cyclohexyl]methanol (500 mg, 1.42 mmol), 6-(trifluoromethyl)pyridine-2-carboxamide (323 mg, 1.70 mmol, CAS #22245-84-7), [2-(2-aminophenyl) phenyl]-methylsulfonyloxy-palladium; ditert-butyl-[2-(2,4,6-triisopropylphenyl)phenyl]phosphane (112 mg, 141 umol), and t-BuOK (317 mg, 2.83 mmol) in dioxane (10 mL) was degassed and purged with N₂ three times. Then the mixture was stirred at 80° C. for 16 hrs under N₂ atmosphere. On completion, the reaction mixture was filtered to give a filtrate, then concentrated in vacuo to give a residue. The residue was purified by reverse phase (0.1% FA condition) to give the title compound (100 mg, 15% yield) as yellow solid. ¹H NMR (400 MHz, DMSO-d₆) δ 10.74 (s, 1H), 8.70 (s, 1H), 8.54-8.38 (m, 2H), 8.34 (s, 1H), 8.23 (d, J=7.2 Hz, 1H), 7.14 (s, 1H), 4.49 (t, J=5.6 Hz, 1H), 4.42-4.30 (m, 1H), 4.20 (q, J=6.4 Hz, 2H), 3.30-3.28 (m, 3H), 2.14 (d, J=10.8 Hz, 2H), 1.96-1.82 (m, 4H), 1.51 (t, J=6.8 Hz, 3H), 1.22-1.07 (m, 2H). LC-MS (ESI+) m/z 463.1 (M+H)⁺.

### Step 4—N-[6-ethoxy-2-(4-formylcyclohexyl)indazol-5-yl]-6-(trifluoromethyl)pyridine-2-carboxamide

To a solution of N-[6-ethoxy-2-[4-(hydroxy methyl)cyclohexyl]indazol-5-yl]-6-(trifluoromethyl) pyridine-2-carboxamide (100 mg, 216 umol) in DCM (2 mL) was added DMP (110 mg, 259 umol). The mixture was then stirred at 20° C. for 1 hr. On completion, the reaction mixture was quenched by saturated aqueous Na₂S₂O₃ (1 ml) and saturated aqueous NaHCO₃ (1 ml). The organic layer was washed by brine (1 ml), dried over anhydrous sodium sulfate and concentrated in vacuo to give the title compound (99 mg, 94% yield) as off-white solid. ¹HNMR (400 MHz, DMSO-d₆) δ 10.73 (s, 1H), 9.64 (s, 1H), 8.70 (s, 1H), 8.50-8.35 (m, 2H), 8.33 (s, 1H), 8.21 (dd, J=1.2, 8.0 Hz, 1H), 7.12 (s, 1H), 4.39-4.37 (m, 1H), 4.19-4.17 (m, 2H), 2.42-2.40 (m, 1H).

### N-[6-cyclopropyl-2-(4-formylcyclohexyl)indazol-5-yl]-6-(trifluoromethyl)pyridine-2-carboxamide (Intermediate CEN)

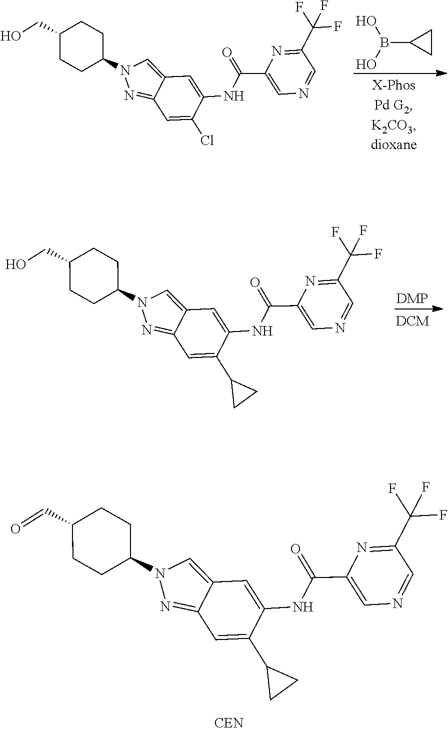

### Step 1—N-[6-cyclopropyl-2-[4-(hydroxymethyl)cyclohexyl]indazol-5-yl]-6-(trifluoromethyl)pyridine-2-carboxamide

A mixture of N-[6-chloro-2-[4-(hydroxymethyl)cyclohexyl]indazol-5-yl]-6-(trifluoromethyl)pyridine-2-carboxamide (300 mg, 662 umol, via Steps 1-3 of Intermediate BPQ), cyclopropylboronic acid (227 mg, 2.65 mmol, CAS #411235-57-9), XPHOS-PD-G₂ (52.1 mg, 66.2 umol) and K₂CO₃ (274 mg, 1.99 mmol) in dioxane (3 mL) was stirred at 90° C. for 16 hrs under N₂. On completion, the reaction mixture was filtered and the filtrate was concentrated in vacuo to give a residue. The residue was purified by prep-HPLC (column: Phenomenex luna C18 250*50 mm*15 um; mobile phase: [water(FA)-ACN]; B %: 48%-78%, 8 min) to give the title compound (140 mg, 46% yield) as white solid. LC-MS (ESI+) m/z 459.1 (M+H)⁺.

### Step 2—N-[6-cyclopropyl-2-(4-formylcyclohexyl)indazol-5-yl]-6-(trifluoromethyl)pyridine-2-carboxamide

To a solution of N-[6-cyclopropyl-2-[4-(hydroxymethyl)cyclohexyl]indazol-5-yl]-6-(trifluoromethyl) pyridine-2-carboxamide (140 mg, 305 umol) in DCM (2 mL) was added DMP (194 mg, 458 umol). The reaction was stirred at 25° C. for 1 hr. On completion, the reaction mixture was quenched with saturated Na₂S₂O₃ (5 mL) and saturated NaHCO₃ (5 mL) at 25° C., and then the reaction mixture was stirred for 30 minutes. On completion, the mixture was extracted with DCM (2×15 mL). Then the combined organic layer was washed with NaHCO₃ (2×15 mL) and washed with saturated salt solution (2×15 mL). The combined organic layer was dried over Na₂SO₄, filtered and the filtrate was concentrated in vacuo to give the title compound (90.0 mg, 64% yield) as brown solid. LC-MS (ESI+) m/z 457.1 (M+H)⁺.

### N-[2-(4-formylcyclohexyl)-6-methoxy-indazol-5-yl]-6-(trifluoromethyl)pyrazine-2-carboxamide (Intermediate CEO)

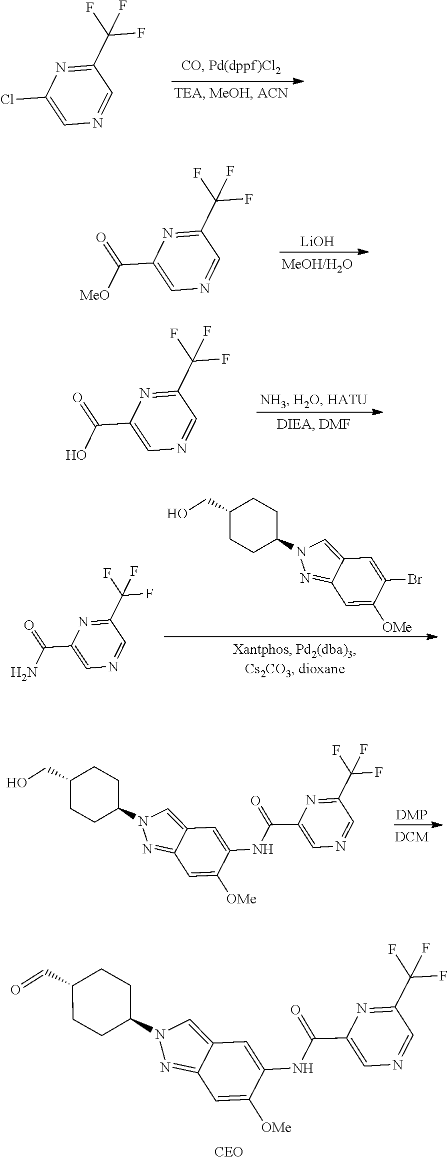

### Step 1—Methyl 6-(trifluoromethyl)pyrazine-2-carboxylate

To a mixture of 2-chloro-6-(trifluoromethyl)pyrazine (2 g, 10.9 mmol, CAS #61655-69-4) in MeOH (20 mL) were added Pd(dppf)Cl₂ (801 mg, 1.10 mmol), and TEA (3.33 g, 32.8 mmol). Then the mixture was stirred at 70° C. for 16 hrs under CO atmosphere (50 psi). On completion, the mixture was filtered and concentrated to give a residue. The residue was purified by column chromatography (SiO₂, Petroleum ether/Ethyl acetate=1:0 to 5:1) to give the title compound (1 g, 44% yield) as a yellow oil. ¹H NMR (400 MHz, CDCl₃) δ 9.51 (s, 1H), 9.15 (s, 1H), 4.08 (s, 3H); LC-MS (ESI+) m/z 206.9 (M+H)⁺.

### Step 2—6-(Trifluoromethyl)pyrazine-2-carboxylic acid

To a solution of methyl 6-(trifluoromethyl)pyrazine-2-carboxylate (500 mg, 2.43 mmol) in H₂O (1 mL) and MeOH (2 mL) was added LiOH·H₂O (203 mg, 4.85 mmol) at 25° C. The reaction mixture was stirred at 25° C. for 0.5 hr. On completion, the mixture was concentrated to give a residue, then 1N HCl was added until the pH=3. The mixture was filtered and the filter cake was dried to give the title compound (465 mg, 99% yield) as a yellow solid.

### Step 3—6-(Trifluoromethyl)pyrazine-2-carboxamide

To a solution of 6-(trifluoromethyl)pyrazine-2-carboxylic acid (465 mg, 2.43 mmol), HATU (1.20 g, 3.15 mmol) and DIEA (940 mg, 7.28 mmol) in DMF (3 mL) was added NH₃·H₂O (10.2 g, 72.7 mmol, 25% solution) in THF (2 mL). The mixture was then stirred at 25° C. for 1 hr. On completion, the residue was diluted with water (20 mL) and extracted with EA (2×20 mL). The combined organic layer was dried over Na₂SO₄, filtered and concentrated in vacuo to give the title compound (237 mg, 48% yield) as a white solid. ¹HNMR (400 MHz, CDCl₃) δ 9.65 (s, 1H), 9.17 (s, 1H), 7.77-7.43 (m, 1H), 5.95 (s, 1H); LC-MS (ESI+) m/z 192.0 (M+H)⁺.

### Step 4—N-[2-[4-(hydroxymethyl)cyclohexyl]-6-methoxy-indazol-5-yl]-6-(trifluoromethyl) pyrazine-2-carboxamide

A mixture of 6-(trifluoromethyl)pyrazine-2-carboxamide (167 mg, 873 umol), [4-(5-bromo-6-methoxy-indazol-2-yl)cyclohexyl]methanol (296 mg, 873 umol, synthesized via Steps 1-3 of Intermediate ATE), 4 Å molecular sieves (873 umol), Cs₂CO₃ (569 mg, 1.75 mmol), Xantphos (101 mg, 174 umol) and Pd₂(dba)₃ (80.0 mg, 87.3 umol) in dioxane (10 mL) was degassed and purged with N₂ three times. Then the mixture was stirred at 100° C. for 16 hrs under N₂ atmosphere. On completion, the mixture was filtered and concentrated to give a residue. The residue was purified by column chromatography (SiO₂, Petroleum ether/Ethyl acetate=0/1) to give the title compound (260 mg, 65% yield) as a yellow solid. ¹H NMR (400 MHz, CDCl₃) δ 10.40 (s, 1H), 9.73 (s, 1H), 9.17 (s, 1H), 8.83 (s, 1H), 7.91 (s, 1H), 7.09 (s, 1H), 4.43-4.24 (m, 1H), 4.03 (s, 3H), 3.57 (d, J=6.0 Hz, 2H), 2.34 (d, J=12.0 Hz, 2H), 2.14-1.90 (m, 4H), 1.72-1.66 (m, 1H), 1.49-1.36 (m, 1H), 1.33-1.20 (m, 2H); LC-MS (ESI+) m/z 450.0 (M+H)⁺.

### Step 5—N-[2-(4-formylcyclohexyl)-6-methoxy-indazol-5-yl]-6-(trifluoromethyl)pyrazine-2-carboxamide

To a mixture of N-[2-[4-(hydroxymethyl)cyclohexyl]-6-methoxy-indazol-5-yl]-6-(trifluoromethyl) pyrazine-2-carboxamide (100 mg, 222 umol) in DCM (5 mL) was added DMP (141 mg, 333 umol). The mixture was then stirred at 25° C. for 1 hr. On completion, the reaction mixture was quenched with saturated NaHCO₃ (2.5 mL) and Na₂S₂O₃ (2.5 mL). Then the mixture was extracted with DCM (3×5 mL). The combined organic layer was washed with NaCl (5 mL×2), dried over Na₂SO₄, filtered and concentrated in vacuo to give the title compound (99.5 mg, 96% yield) as a yellow solid. ¹H NMR (400 MHz, CDCl₃) δ 10.39 (s, 1H), 9.72 (s, 2H), 9.16 (s, 1H), 8.82 (s, 1H), 7.89 (s, 1H), 7.08 (s, 1H), 4.43-4.26 (m, 1H), 4.02 (s, 3H), 2.48-2.34 (m, 3H), 2.32-2.21 (m, 2H), 2.13-2.02 (m, 2H), 1.56-1.48 (m, 2H); LC-MS (ESI+) m/z 448.1 (M+H)⁺.

### N-[2-(4-formylcyclohexyl)-6-methoxy-indazol-5-yl]-2-(trifluoromethyl)pyrimidine-4-carboxamide (Intermediate CEP)

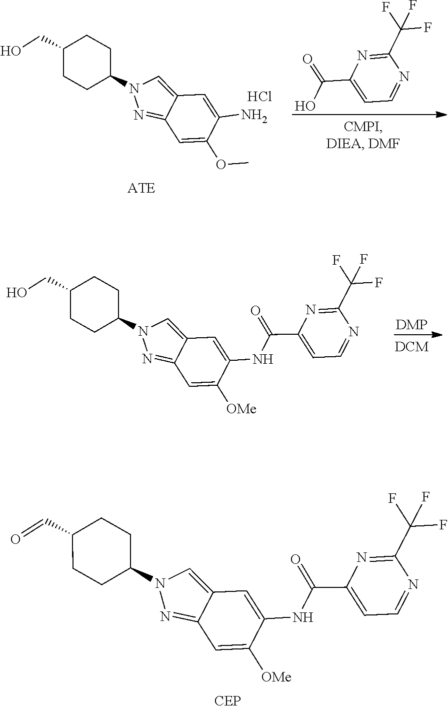

### Step 1—N-[2-[4-(hydroxymethyl)cyclohexyl]-6-methoxy-indazol-5-yl]-2-(trifluoromethyl) pyrimidine

A mixture of 2-(trifluoromethyl)pyrimidine-4-carboxylic acid (50.9 mg, 265 umol, HCl, CAS #878742-59-7), CMPI (88 mg, 344 umol) and DIPEA (102 mg, 795 umol) in DMF (0.5 mL) was stirred at 25° C. for 15 min. Then a solution of [4-(5-amino-6-methoxy-indazol-2-yl)cyclohexyl]methanol (73 mg, 265 umol, Intermediate ATE) in DMF (0.5 mL) was added. The reaction mixture was stirred at 25° C. for 0.5 hour. On completion, the reaction mixture was quenched with H₂O (0.1 mL). The residue was purified by reversed phase flash (0.1% FA condition) to give the title compound (93 mg, 71% yield) as a white solid. LC-MS (ESI+) m/z 450.1 (M+H)⁺.

### Step 2—N-[2-(4-formylcyclohexyl)-6-methoxy-indazol-5-yl]-2-(trifluoromethyl)pyrimidine-4-carboxamide

To a mixture of N-[2-[4-(hydroxymethyl)cyclohexyl]-6-methoxy-indazol-5-yl]-2-(trifluoromethyl) pyrimidine-4-carboxamide (80 mg, 178 umol) in DCM (2 mL) was added DMP (90.6 mg, 213 umol). The mixture was then stirred at 25° C. for 2 hrs. On completion, the reaction mixture was quenched with sat. Na₂S₂O₃ solution (2 mL) and sat. NaHCO₃ solution (2 mL) under stirring. Then the mixture was extracted with DCM (2×5 mL). The combined organic layer was dried over Na₂SO₄, filtered and concentrated in vacuo to give the title compound (100 mg, 98% yield) as a yellow solid. LC-MS (ESI+) m/z 447.9 (M+H)⁺.

### 3-(3-cyclopropyl-2-oxo-4-piperazin-1-yl-benzimidazol-1-yl)piperidine-2,6-dione (Intermediate CEQ)

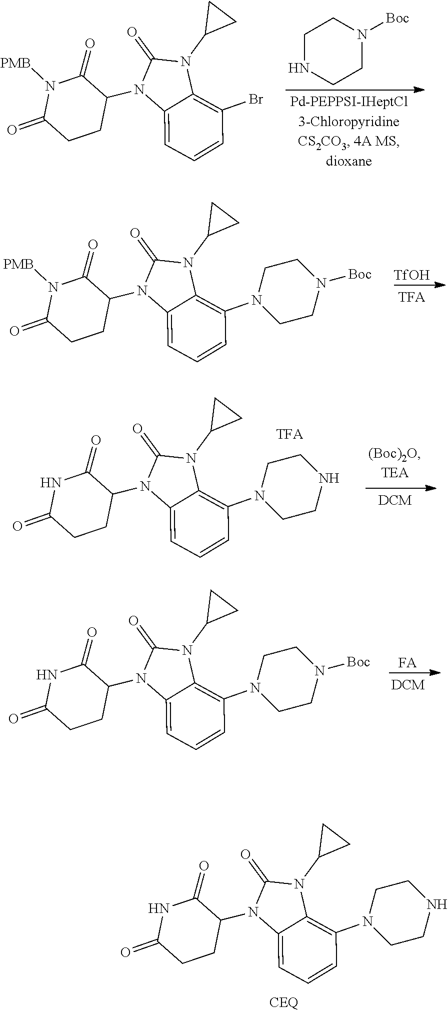

### Step 1—Tert-butyl 4-[3-cyclopropyl-1-[1-[(4-methoxyphenyl)methyl]-2,6-dioxo-3-piperidyl]-2-oxo-benzimidazol-4-yl]piperazine-1-carboxylate

A mixture of 3-(4-bromo-3-cyclopropyl-2-oxo-benzimidazol-1-yl)-1-[(4-methoxyphenyl)methyl]piperidine-2,6-dione (300 mg, 619 umol, synthesized via Steps 1-4 of Intermediate CGN), tert-butyl piperazine-1-carboxylate (173 mg, 929 umol), Pd-PEPPSI-IHeptCl 3-Chloropyridine (40.0 mg, 61.9 umol), Cs₂CO₃ (403 mg, 1.24 mmol) and 4 Å molecular sieves (50 mg) in dioxane (6 mL) was stirred at 100° C. for 16 hrs. On completion, the reaction was filtered and concentrated to give a residue. The residue was purified by reverse phase (FA condition) to give the title compound (500 mg, 68.4% yield) as yellow solid. ¹H NMR (400 MHz, DMSO-d₆) δ 7.20 (d, J=8.4 Hz, 2H), 7.02-6.94 (m, 2H), 6.85 (d, J=8.4 Hz, 3H), 6.03-5.92 (m, 1H), 5.53 (dd, J=5.2, 12.8 Hz, 1H), 5.05 (dd, J=1.2, 10.4 Hz, 1H), 4.88-4.79 (m, 2H), 4.79-4.69 (m, 3H), 4.01-3.86 (m, 2H), 3.72 (s, 3H), 3.12-2.93 (m, 3H), 2.90 (d, J=10.4 Hz, 2H), 2.83 (d, J=2.4 Hz, 1H), 2.81-2.69 (m, 3H), 2.11-2.02 (m, 1H), 1.42 (s, 9H); LC-MS (ESI+) m/z 590.4 (M+H)⁺.

### Step 2—3-(3-Cyclopropyl-2-oxo-4-piperazin-1-yl-benzimidazol-1-yl)piperidine-2,6-dione

To a solution of tert-butyl 4-[3-cyclopropyl-1-[1-[(4-methoxyphenyl)methyl]-2,6-dioxo-3-piperidyl]-2-oxo-benzimidazol-4-yl]piperazine-1-carboxylate (120 mg, 203 umol) in TFA (1.5 mL) was added TfOH (127 mg, 849 umol), then the mixture was stirred at 70° C. for 1 hr. On completion, the reaction mixture was concentrated in vacuo to give title compound (65 mg, 66% yield, TFA) as black brown liquid. LC-MS (ESI+) m/z 370.2 (M+H)⁺.

### Step 3—Tert-butyl 4-[3-cyclopropyl-1-(2,6-dioxo-3-piperidyl)-2-oxo-benzimidazol-4-yl]piperazine-1-carboxylate

To a solution of 3-(3-cyclopropyl-2-oxo-4-piperazin-1-yl-benzimidazol-1-yl)piperidine-2,6-dione (40.0 mg, 82.7 umol, TFA) in DCM (1 mL) was added TEA (25.1 mg, 248 umol) and (Boc)₂O (27.0 mg, 124 umol), then the mixture was stirred at 25° C. for 2 hrs. On completion, the reaction mixture was concentrated in vacuo to give the residue. The residue was purified by reversed-phase (0.10% FA condition) to give the title compound (35 mg, 90.0% yield) as a white solid. LC-MS (ESI+) m/z 470.3 (M+H)⁺.

### Step 4—3-(3-Cyclopropyl-2-oxo-4-piperazin-1-yl-benzimidazol-1-yl)piperidine-2,6-dione

To a solution of tert-butyl 4-[3-cyclopropyl-1-(2,6-dioxo-3-piperidyl)-2-oxo-benzimidazol-4-yl]piperazine-1-carboxylate (35 mg, 74.5 umol) in DCM (0.20 mL) was added HCOOH (3.58 mg, 74.5 umol), then the mixture was stirred at 25° C. for 1 hr. On completion, the reaction mixture was concentrated in vacuo to give the title compound (25 mg, 80% yield, FA) as black brown liquid. LC-MS (ESI+) m/z 370.3 (M+H)⁺.

### 3-[4-(3,3-Dimethylpiperazin-1-yl)-3-methyl-2-oxo-benzimidazol-1-yl]-1-[(4-methoxyphenyl) methyl]piperidine-2,6-dione (Intermediate CER)

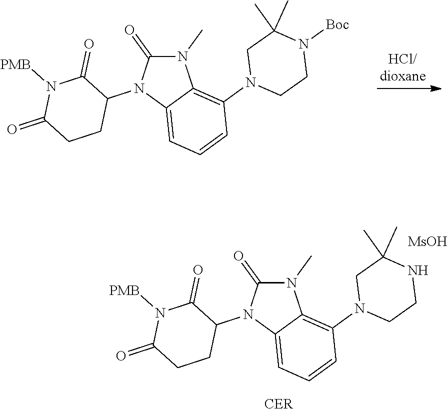

To a solution of tert-butyl-4-[1-[1-[(4-methoxyphenyl)methyl]-2,6-dioxo-3-piperidyl]-3-methyl-2-oxo-benzimidazol-4-yl]-2,2-dimethyl-piperazine-1-carboxylate (230 mg, 388 umol, synthesized via Step 1 of Intermediate CBC) in DCM (2 mL) was added HCl/dioxane (4 M, 97.1 uL). Then the reaction mixture was stirred at 25° C. for 0.5 hr. On completion, the reaction mixture was concentrated in vacuo to give the title compound (205 mg, 100% yield, HCl salt) as a yellow solid. LC-MS (ESI⁺) m/z 492.2 (M+H)⁺.

### N-[2-[4-(iodomethyl)cyclohexyl]-6-methyl-indazol-5-yl]-6-(trifluoromethyl)pyridine-2-carboxamide (Intermediate CES)

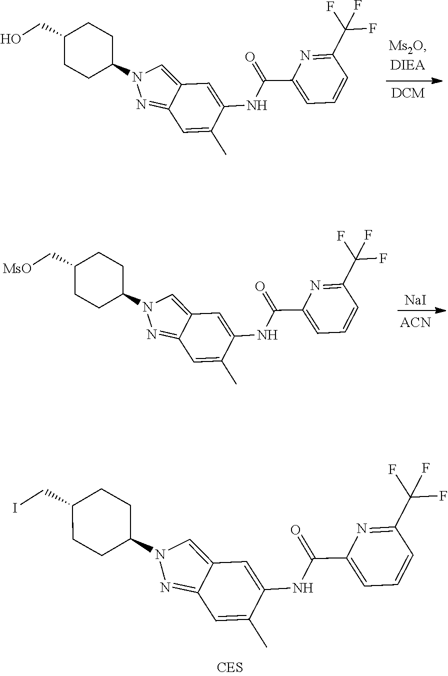

### Step 1—[4-[6-Methyl-5-[[6-(trifluoromethyl)pyridine-2-carbonyl]amino]indazol-2-yl]cyclohexyl]methyl methanesulfonate

To a solution of N-[2-[4-(hydroxymethyl)cyclohexyl]-6-methyl-indazol-5-yl]-6-(trifluoromethyl) pyridine-2-carboxamide (500 mg, 1.16 mmol, synthesized via Step 1 of Intermediate BSC) in DCM (8 mL) was added DIEA (448 mg, 3.47 mmol, 604 uL), then methylsulfonyl methanesulfonate (402 mg, 2.31 mmol) was added at 0° C. Then the reaction mixture was stirred at 25° C. for 2 hrs. On completion, the mixture was diluted with water (15 mL) and extracted with DCM (2×20 mL). The combined organic layers were washed with brine (15 mL), dried over Na₂SO₄, filtered and concentrated in vacuo to give the title compound (580 mg, 98% yield) as a yellow solid. LC-MS (ESI⁺) m/z 511.1 (M+H)⁺.

### Step 2—N-[2-[4-(iodomethyl)cyclohexyl]-6-methyl-indazol-5-yl]-6-(trifluoromethyl)pyridine-2-carboxamide

To a solution of [4-[6-methyl-5-[[6-(trifluoromethyl)pyridine-2-carbonyl]amino]indazol-2-yl]cyclohexyl]methyl methanesulfonate (484 mg, 949 umol) in ACN (5 mL) was added NaI (640 mg, 4.27 mmol), then the reaction mixture was stirred at 70° C. for 16 hrs. On completion, the mixture was diluted with water (10 mL) and extracted with DCM (2×20 mL). The combined organic layers were washed with brine (10 mL), dried over Na₂SO₄, filtered and concentrated in vacuo to give a residue. The residue was purified by reverse phase (0.1% FA condition) to give the title compound (500 mg, 97% yield) as brown solid. ¹H NMR (400 MHz, DMSO-d₆) δ 10.12 (s, 1H), 8.45-8.41 (m, 1H), 8.39 (d, J=7.6 Hz, 1H), 8.36 (s, 1H), 8.23-8.17 (m, 2H), 7.51 (s, 1H), 4.51-4.32 (m, 1H), 3.30 (d, J=6.0 Hz, 2H), 2.40 (s, 3H), 2.21-2.10 (m, 2H), 2.05-1.87 (m, 4H), 1.62-1.47 (m, 1H), 1.33-1.16 (m, 2H), LC-MS (ESI⁺) m/z 543.0 (M+H)⁺.

### 5-Bromo-4-hydroxy-2-nitro-benzaldehyde (Intermediate AWN)

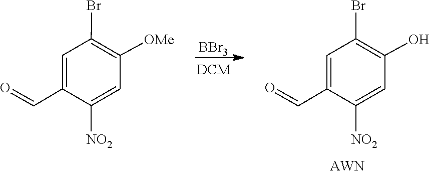

To a mixture of 5-bromo-4-methoxy-2-nitro-benzaldehyde (7.20 g, 27.6 mmol, synthesized via Steps 1-2 of Intermediate ATE) in DCM (100 mL) was added BBr₃ (20.8 g, 83.0 mmol). The reaction mixture was stirred at −70° C. to 25° C. for 12 hours. On completion, the reaction mixture was quenched with MeOH (10 mL) and concentrated in vacuo. The residue was triturated with DCM/PE=1/2, filtered and concentrated in vacuo to give to the title compound (4.70 g, 69% yield) as yellow solid. ¹H NMR (400 MHz, DMSO-d₆) δ 12.63-12.10 (m, 1H), 10.02 (s, 1H), 8.08 (s, 1H), 7.52 (s, 1H).

### N-[2-(4-formylcyclohexyl)-6-methylsulfanyl-indazol-5-yl]-6-(trifluoromethyl)pyridine-2-carboxamide (Intermediate BIZ)

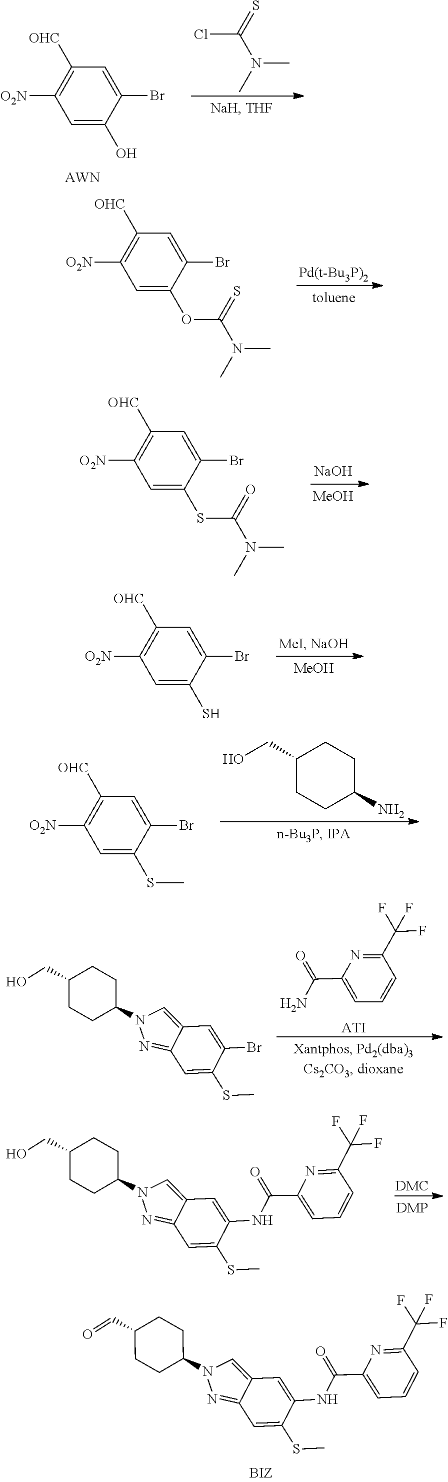

### Step 1—0-(2-bromo-4-formyl-5-nitro-phenyl) N,N-dimethylcarbamothioate

To a solution of 5-bromo-4-hydroxy-2-nitro-benzaldehyde (5.00 g, 20.3 mmol, Intermediate AWN) in THF (250 mL) was added NaH (894 mg, 22.3 mmol, 60% dispersion in mineral oil) at 0° C. for 0.5 hour. Then the mixture was stirred at 25° C. for 1 hour. Then N,Ndimethylcarbamothioylchloride (3.01 g, 24.4 mmol, CAS #16420-13-6) in THF (100 mL) was added at 0° C. Then the mixture was stirred 25° C. for 12 hours. On completion, the reaction mixture was diluted in H₂O (100 mL) and EA (300 mL). The organic phase was separated, washed with brine (100 mL), dried over Na₂SO₄, filtered and concentrated. The residue was purified by column chromatography (petroleum ether/ethyl acetate=10/1 to 0/1) to give the title compound (13.0 g, 64% yield) as a yellow solid. ¹H NMR (400 MHz, CDCl₃) δ10.43 (s, 1H), 8.21 (s, 1H), 7.98 (s, 1H), 3.50 (s, 3H), 3.46 (s, 3H).

### Step 2—S-(2-bromo-4-formyl-5-nitro-phenyl) N,N-dimethylcarbamothioate

A mixture of O-(2-bromo-4-formyl-5-nitro-phenyl) N,N-dimethylcarbamothioate (13 g, 39.0 mmol), Pd(t-Bu₃P)₂ (399 mg, 780 umol) in toluene (200 mL) was degassed and purged with N₂ for 3 times, and then the mixture was stirred at 120° C. for 24 hours under N₂ atmosphere. On completion, the reaction mixture was concentrated under reduced pressure to remove toluene. The residue was diluted with H₂O (200 mL) and extracted with DCM (400 mL×2). The combined organic layers were washed with brine (200 mL), dried over Na₂SO₄, filtered and concentrated under reduced pressure to give a residue. The residue was triturated with EA (20 mL) to give the title compound (7.00 g, 53% yield) as a yellow solid. ¹HNMR (400 MHz, CDCl₃) δ 10.42 (s, 1H), 8.47 (s, 1H), 8.18 (s, 1H), 3.17 (s, 3H), 3.09 (s, 3H).

### Step 3—5-Bromo-2-nitro-4-sulfanyl-benzaldehyde

A mixture of S-(2-bromo-4-formyl-5-nitro-phenyl) N,N-dimethylcarbamothioate (2.00 g, 6.00 mmol), NaOH (480 mg, 12.0 mmol) in MeOH (80 mL) was degassed and purged with N₂ for 3 times, and then the mixture was stirred at 25° C. for 12 hours under N₂ atmosphere. On completion, the reaction mixture was quenched by addition HCl (1M) and adjusted to pH=5-6 at 25° C., and then filtered. The filter cake was dried in vacuo to give the title compound (1.50 g, 90% yield) as a brown solid.

### Step 4—5-Bromo-4-methylsulfanyl-2-nitro-benzaldehyde

To a solution of 5-bromo-2-nitro-4-sulfanyl-benzaldehyde (1.5 g, 5.72 mmol), NaOH (458 mg, 11.5 mmol) in MeOH (80 mL) was stirred for 0.5 hr. Then iodomethane (4.06 g, 28.6 mmol) was added at 25° C. and the mixture was stirred at 25° C. for 3 hours. On completion, the reaction mixture was quenched by addition HCl (1 M) to adjust the pH=7-8 at 0° C., and then the reaction mixture was concentrated under reduced pressure to remove MeOH. The residue was diluted with H₂O (20 mL) and extracted with EA (2×200 mL). The combined organic layers were washed with brine (100 mL), dried over Na₂SO₄, filtered and concentrated in vacuo to give a residue. The residue was purified by column chromatography (petroleum ether/ethyl acetate=10/1 to 1/1) to give the title compound (1.50 g, 94% yield) as a yellow solid. ¹HNMR (400 MHz, CDCl₃) δ 10.34 (s, 1H), 8.12 (s, 1H), 7.74 (s, 1H), 2.62 (s, 3H).

### Step 5—[4-(5-Bromo-6-methylsulfanyl-indazol-2-yl)cyclohexyl]methanol

A mixture of 5-bromo-4-methylsulfanyl-2-nitro-benzaldehyde (1.30 g, 4.71 mmol) and (4-aminocyclohexyl) methanol (608 mg, 4.71 mmol, CAS #1467-84-1) in IPA (45 mL) was stirred at 80° C. for 16 hours under N₂. The mixture was cooled to 25° C. and tributylphosphane (2.86 g, 14.1 mmol) was added. The reaction mixture was stirred at 80° C. for 6 hours. On completion, the reaction mixture was partitioned between H₂O (100 mL) and EA (400 mL). The organic phase was separated, washed with brine (100 mL), dried over Na₂SO₄, filtered and concentrated in vacuo. The residue was purified by column chromatography (petroleum ether/ethyl acetate=10/1 to 1/1) to give the title compound (1.10 g, 65% yield) as a yellow solid. ¹H NMR (400 MHz, CDCl₃) δ 7.85 (d, J=6.8 Hz, 2H), 7.39 (s, 1H), 4.36-430 (m, 1H), 3.54 (s, 2H), 2.50 (s, 3H), 2.30 (d, J=10.8 Hz, 2H), 2.05-1.94 (m, 4H), 1.67-1.65 (m, 1H), 1.44-1.43 (m, 1H), 1.28-1.19 (m, 2H); LC-MS (ESI+) m/z 357.0 (M+H)⁺.

### Step 6—N-[2-[4-(hydroxymethyl)cyclohexyl]-6-methylsulfanyl-indazol-5-yl]-6-(trifluoromethyl) pyridine-2-carboxamide

A mixture of [4-(5-bromo-6-methylsulfanyl-indazol-2-yl)cyclohexyl]methanol (1.00 g, 2.81 mmol), 6-(trifluoromethyl)pyridine-2-carboxamide (535 mg, 2.81 mmol, Intermediate ATI), Cs₂CO₃ (1.83 g, 5.63 mmol), Xantphos (326 mg, 563 umol) and Pd₂(dba)₃ (258 mg, 281 umol) in dioxane (15 mL) was degassed and purged with N₂ for 3 times, and then the mixture was stirred at 95° C. for 24 hours under N₂ atmosphere. The reaction mixture was filtered and concentrated under reduced pressure to give a residue. The residue was purified by column chromatography (petroleum ether/ethyl acetate=20/1 to 1/1) to give the title compound (700 mg, 46% yield) as a brown solid. ¹HNMR (400 MHz, CDCl₃) δ 11.04 (s, 1H), 8.76 (s, 1H), 8.43 (d, J=8.0 Hz, 1H), 8.06 (t, J=8.0 Hz, 1H), 7.88 (d, J=7.2 Hz, 2H), 7.81 (d, J=7.2 Hz, 1H), 4.35-4.27 (m, 1H), 3.49 (d, J=6.0 Hz, 2H), 2.42 (s, 3H), 2.29-2.25 (m, 2H), 2.00-1.90 (m, 4H), 1.65-1.56 (m, 2H), 1.24-1.14 (m, 2H).

### Step 7—N-[2-(4-formylcyclohexyl)-6-methylsulfanyl-indazol-5-yl]-6-(trifluoromethyl)pyridine-2-carboxamide

To a solution of N-[2-[4-(hydroxymethyl)cyclohexyl]-6-methylsulfanyl-indazol-5-yl]-6-(trifluoromethyl) pyridine-2-carboxamide (120 mg, 258 umol) in DCM (2 mL) was added DMP (142 mg, 336 umol) at 0° C. The mixture was stirred at 0-25° C. for 1 hour. On completion, the reaction mixture was quenched by addition of Na₂S₂O₃ (1 mL). Then NaHCO₃ (1 mL) was added until the pH=8 at 25° C. The mixture was next diluted with H₂O (5 mL) and extracted with DCM (2×10 mL). The combined organic layers were dried over Na₂SO₄, filtered and concentrated in vacuo to give the title compound (100 mg, 84% yield) as a yellow solid. LC-MS (ESI+) m/z 463.2 (M+H)⁺.

### N-[2-(4-formylcyclohexyl)-6-methylsulfonyl-indazol-5-yl]-6-(trifluoromethyl)pyridine-2-carboxamide (Intermediate BJB)

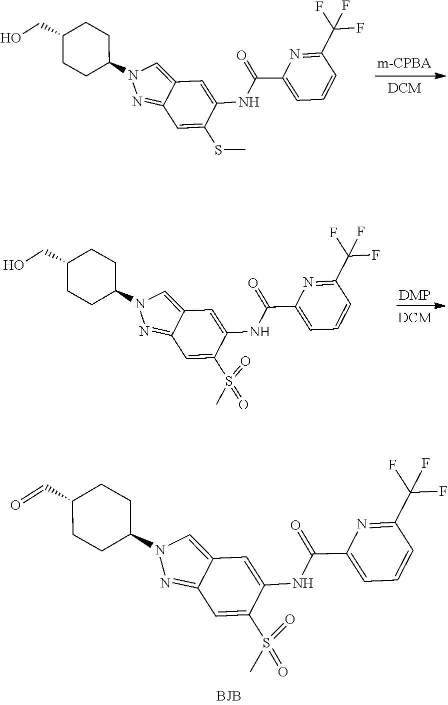

### Step 1—N-[2-[4-(hydroxymethyl)cyclohexyl]-6-methylsulfonyl-indazol-5-yl]-6-(trifluoromethyl) pyridine-2-carboxamide

To a solution of N-[2-[4-(hydroxymethyl)cyclohexyl]-6-methylsulfanyl-indazol-5-yl]-6-(trifluoromethyl)pyridine-2-carboxamide (400 mg, 861 umol, synthesized via Steps 1-6 of Intermediate BIZ) in DCM (15 mL) was added m-CPBA (928 mg, 4.31 mmol, 80% solution) at 0° C. The mixture was then stirred at 25° C. for 12 hours. On completion, the reaction mixture was concentrated in vacuo to give a residue. The residue was purified by column chromatography (petroleum ether/ethyl acetate=10/1 to 3/1) to give the title compound (300 mg, 56% yield) as a yellow solid. LC-MS (ESI+) m/z 497.1 (M+H)⁺.

### Step 2—N-[2-(4-formylcyclohexyl)-6-methylsulfonyl-indazol-5-yl]-6-(trifluoromethyl)pyridine-2-carboxamide

To a solution of N-[2-[4-(hydroxymethyl)cyclohexyl]-6-methylsulfonyl-indazol-5-yl]-6-(trifluoromethyl)pyridine-2-carboxamide (70.0 mg, 140 umol) in DCM (2 mL) was added DMP (77.7 mg, 183 umol). The mixture was stirred at 0-25° C. for 1 hour. The reaction mixture was quenched by addition saturated NaHCO₃ (1 mL) and Na₂S₂O₃ (1 mL), and then diluted with H₂O (5 mL) and extracted with DCM (2×25 mL). The combined organic layers were washed with brine (10 mL), dried over Na₂SO₄, filtered and concentrated in vacuo to give the title compound (60.0 mg, 86% yield) as a yellow solid. LC-MS (ESI+) m/z 495.1 (M+H)⁺.

### N-[2-(4-formylcyclohexyl)-6-methylsulfinyl-indazol-5-yl]-6-(trifluoromethyl)pyridine-2-carboxamide (Intermediate CEU)

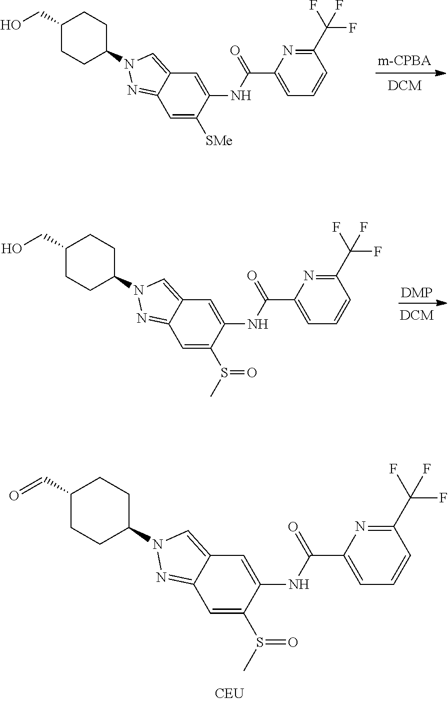

### Step 1—N-[2-[4-(hydroxymethyl)cyclohexyl]-6-methylsulfinyl-indazol-5-yl]-6-(trifluoromethyl) pyridine-2-carboxamide

To a solution of N-[2-[4-(hydroxymethyl)cyclohexyl]-6-methylsulfanyl-indazol-5-yl]-6-(trifluoromethyl)pyridine-2-carboxamide (200 mg, 430 umol, synthesized via Steps 1-6 of Intermediate BIZ) in DCM (2 mL) was added a mixture of m-CPBA (111 mg, 517 umol, 80% solution) in DCM (2 mL) dropwise at −10° C., then the mixture was stirred at −10° C. for 0.5 hr. On completion, the mixture was diluted with DCM (10 mL), then quenched with saturated NaHCO₃(10 mL) and saturated Na2S203 (10 mL), and the mixture was stirred at 25° C. for 0.5 hr. After that, the organic layer was washed with saturated NaHCO₃(10 mL×3), the organic layer was separated and washed with saturated NaCl (20 mL). The organic layer was dried over Na2SO4, filtered and concentrated in vacuo to give the title compound (160 mg, 77% yield) as yellow solid. ¹H NMR (400 MHz, DMSO-d₆) δ 11.4 (s, 1H), 8.59 (s, 1H), 8.44 (s, 1H), 8.43-8.34 (m, 2H), 8.19 (d, J=7.6 Hz, 1H), 8.02 (s, 1H), 4.55-4.47 (m, 2H), 3.31-3.27 (m, 2H), 2.86 (s, 3H), 2.17 (d, J=9.2 Hz, 2H), 1.93 (d, J=10.4 Hz, 4H), 1.55-1.45 (m, 1H), 1.23 (s, 2H); LC-MS (ESI⁺) m/z 481.1 (M+H)⁺.

### Step 2—N-[2-(4-formylcyclohexyl)-6-methylsulfinyl-indazol-5-yl]-6-(trifluoromethyl)pyridine-2-carboxamide

To a solution of N-[2-[4-(hydroxymethyl)cyclohexyl]-6-methylsulfinyl-indazol-5-yl]-6-(trifluoromethyl)pyridine-2-carboxamide (160 mg, 333 umol) in DCM (2 mL) was added DMP (169 mg, 399 umol), then the mixture was stirred at 25° C. for 0.5 hr. On completion, the mixture was diluted with DCM (20 mL), then quenched with saturated NaHCO₃(10 mL) and saturated Na2S203 (10 mL). The mixture was stirred at 25° C. for 0.5 hr. Next, the organic layer was washed with saturated NaHCO₃(20 mL×3), the organic layer was separated and washed with saturated NaCl (10 mL). The organic layer was dried over Na₂SO₄, filtered and concentrated in vacuo to give the title compound (140 mg, 88% yield) as yellow solid. ¹H NMR (400 MHz, DMSO-d₆) δ 11.46 (s, 1H), 9.65 (s, 1H), 8.59 (s, 1H), 8.45 (s, 1H), 8.44-8.35 (m, 2H), 8.19 (d, J=7.6 Hz, 1H), 8.03 (s, 1H), 4.61-4.50 (m, 1H), 2.86 (s, 3H), 2.47-2.40 (m, 1H), 2.26-2.19 (m, 2H), 2.17-2.09 (m, 2H), 2.07-1.97 (m, 2H), 1.47-1.42 (m, 2H); LC-MS (ESI⁺) m/z 479.0 (M+H)⁺.

### Methyl 7-cyano-4-(isopropylamino)-[1,4]benzodioxino[2,3-b]pyridine-3-carboxylate (Intermediate CEV) and methyl 8-cyano-4-(isopropylamino)-[1,4]benzodioxino[2,3-b]pyridine-3-carboxylate (Intermediate CEW)

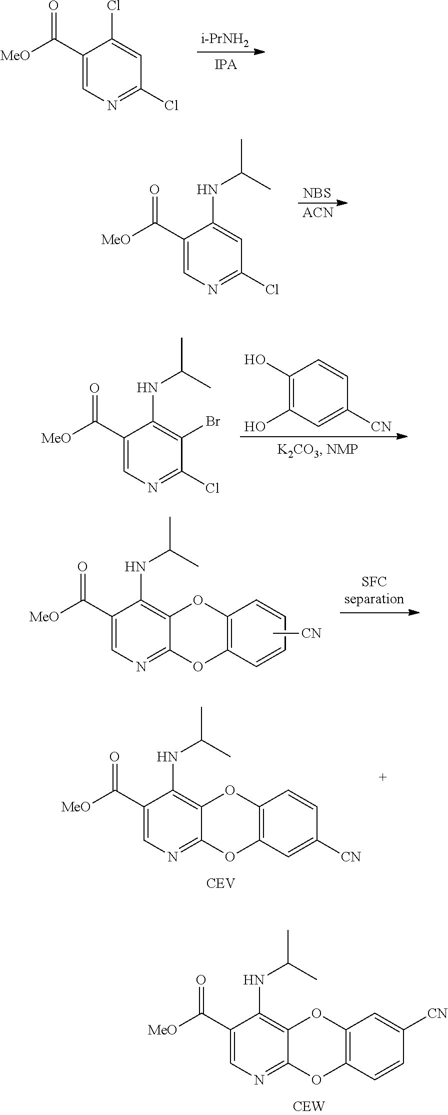

### Step 1—Methyl 6-chloro-4-(isopropylamino)pyridine-3-carboxylate

To a solution of methyl 4,6-dichloropyridine-3-carboxylate (6.50 g, 31.5 mmol) and propan-2-amine (1.86 g, 31.5 mmol, 2.71 mL, CAS #65973-52-6) in IPA (80 mL) was added DIEA (20.3 g, 157 mmol, 27.4 mL), then the reaction mixture was stirred at 50° C. for 16 hrs. On completion, the reaction mixture was concentrated in vacuo to give a residue. The residue was purified by column chromatography (SiO₂, PE:EA=100:1 to PE:EA=15:1, P1:Rf=0.22) to give the title compound (4.40 g, 61% yield) as a white solid. ¹H NMR (400 MHz, DMSO-d₆) δ 8.51 (s, 1H), 7.96 (d, J=8.0 Hz, 1H), 6.83 (s, 1H), 3.90-3.84 (m, 1H), 3.82 (s, 3H), 1.20 (s, 3H), 1.19 (s, 3H). LC-MS (ESI⁺) m/z 228.9 (M+H)⁺.

### Step 2—Methyl 5-bromo-6-chloro-4-(isopropylamino)pyridine-3-carboxylate

To a solution of methyl 6-chloro-4-(isopropylamino)pyridine-3-carboxylate (4.10 g, 17.9 mmol) in ACN (10 mL) was added NBS (3.19 g, 17.9 mmol), then the reaction mixture was stirred at 25° C. for 4 hrs. On completion, the reaction mixture was filtered and concentrated in vacuo to give a residue. The residue was diluted with water (40 mL) and extracted with EA (2×80 mL). The combined organic layers were washed with brine (30 mL), dried over Na₂SO₄, filtered and the filtrate was concentrated in vacuo to give the title compound (5.50 g, 100% crude yield) as white solid. ¹H NMR (400 MHz, DMSO-d₆) δ 11.05 (s, 1H), 7.50 (d, J=8.4 Hz, 1H), 4.39-4.21 (m, 1H), 3.86 (s, 3H), 1.20 (s, 3H), 1.19 (s, 3H), LC-MS (ESI⁺) m/z 309.1 (M+H)⁺.

### Step 3—Methyl 8-cyano-4-(isopropylamino)-[1,4]benzodioxino[2,3-b]pyridine-3-carboxylate

To a solution of methyl 5-bromo-6-chloro-4-(isopropylamino)pyridine-3-carboxylate (4.80 g, 15.6 mmol) and 3,4-dihydroxybenzonitrile (2.32 g, 17.1 mmol, CAS #17345-61-8) in NMP (50 mL) was added K₂CO₃ (6.47 g, 46.8 mmol). Then the reaction mixture was stirred at 130° C. for 2 hrs. On completion, the reaction mixture was filtered and concentrated in vacuo to give a residue. The residue was diluted with water (250 mL) and extracted with EA (2×50 mL). The combined organic layers were washed with brine (2×50 mL) and dried over Na₂SO₄, filtered and the filtrate was concentrated in vacuo to give a residue. The water (20 mL) was added slowly to the residue while stirring. The precipitated white solid was isolated by filtration and the residue was washed with water. The crude product was triturated with PE:EA=(10:1) to give the title compound (1.00 g, 20% yield) as a white solid. LC-MS (ESI⁺) m/z 326.2 (M+H)⁺.

### Step 4—Methyl 7-cyano-4-(isopropylamino)-[1,4]benzodioxino[2,3-b]pyridine-3-carboxylate and methyl 8-cyano-4-(isopropylamino)-[1,4]benzodioxino[2,3-b]pyridine-3-carboxylate

Methyl 8-cyano-4-(isopropylamino)-[1,4]benzodioxino[2,3-b]pyridine-3-carboxylate (1.00 g, 3.07 mmol) was separated by SFC (column: DAICEL CHIRALPAK AD (250 mm*30 mm, 10 um); mobile phase: [0.1% NH3H2O MEOH]; B %: 25%-25%, 3; 45 min) to give methyl 7-cyano-4-(isopropylamino)-[1,4]benzodioxino[2,3-b]pyridine-3-carboxylate (250 mg, 25% yield) as a white solid (¹HNMR (400 MHz, DMSO-d₆) δ 8.38 (s, 1H), 8.23 (d, J=7.2 Hz, 1H), 7.30-7.27 (m, 1H), 7.23 (d, J=1.6 Hz, 1H), 6.91 (d, J=8.0 Hz, 1H), 4.48-4.32 (m, 1H), 3.88 (s, 3H), 1.32 (s, 3H), 1.31 (s, 3H) and methyl 8-cyano-4-(isopropylamino)-[1,4]benzodioxino[2,3-b]pyridine-3-carboxylate (500 mg, 50% yield) as a white solid (¹H NMR (400 MHz, DMSO-d₆) δ 8.37 (s, 1H), 8.23 (d, J=7.6 Hz, 1H), 7.30-7.27 (m, 1H), 7.13 (d, J=1.6 Hz, 1H), 7.04 (d, J=8.4 Hz, 1H), 4.51-4.30 (m, 1H), 3.88 (s, 3H), 1.33 (s, 3H), 1.31 (s, 3H). LC-MS (ESI⁺) m/z 195.1 (M+H)⁺).

### 7-Cyano-4-(isopropylamino)-[1,4]benzodioxino[2,3-b]pyridine-3-carboxylic acid (Intermediate CEX)

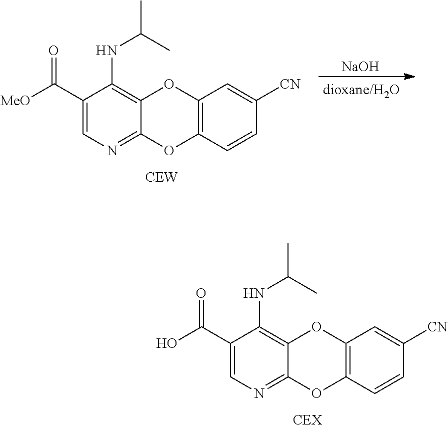 [SMILES: COC(=O)c1cnc2c(c1NC(C)C)Oc1cc(C#N)ccc1O2 ; "NaOH" ; CC(C)Nc1c(C(=O)O)cnc2c1Oc1cc(C#N)ccc1O2]

To a solution of methyl 7-cyano-4-(isopropylamino)-[1,4]benzodioxino[2,3-b]pyridine-3-carboxylate (15.0 mg, 46.1 umol, Intermediate CEW) in dioxane (0.5 mL) and H₂O (0.15 mL) was added NaOH (7.38 mg, 184 umol), then the reaction mixture was stirred at 25° C. for 8 hrs. On completion, the reaction mixture was filtered and the filtrate was concentrated in vacuo to give a residue. The residue was diluted with water (5 mL) and acidified with citric acid until the pH=5-6, then the residue was extracted with EA (2×5 mL). The combined organic layers were washed with brine (5 mL), dried over Na₂SO₄, filtered and the filtrate was concentrated in vacuo to give the title compound (140 mg, 98% yield) as a white solid, LC-MS (ESI⁺) m/z 312.2 (M+H)⁺.

### 3-[4-[1-[(4-Aminocyclohexyl)methyl]-4-piperidyl]-3-methyl-2-oxo-benzimidazol-1-yl]piperidine-2,6-dione (Intermediate CEY)

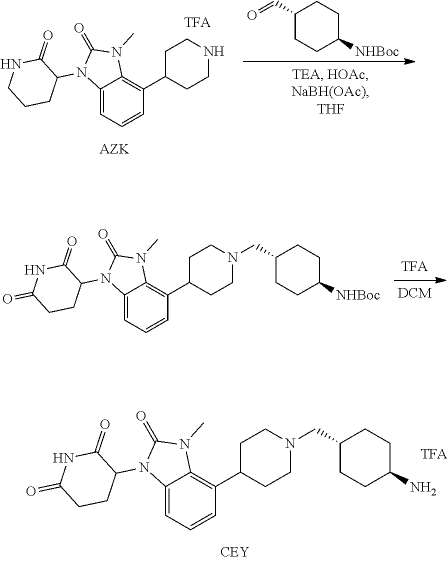 [SMILES: Cn1c(=O)n(C2CCCNC2=O)c2cccc(C3CCNCC3)c21 ; CC(C)(C)OC(=O)N[C@H]1CC[C@H](C=O)CC1 ; Cn1c(=O)n(C2CCC(=O)NC2=O)c2cccc(C3CCN(C[C@H]4CC[C@H](NC(=O)OC(C)(C)C)CC4)CC3)c21 ; Cn1c(=O)n(C2CCC(=O)NC2=O)c2cccc(C3CCN(C[C@H]4CC[C@H](N)CC4)CC3)c21]

### Step 1—Tert-butyl N-[4-[[4-[1-(2,6-dioxo-3-piperidyl)-3-methyl-2-oxo-benzimidazol-4-yl]-1-piperidyl] methyl]cyclohexyl]carbamate

To a solution of 3-[3-methyl-2-oxo-4-(4-piperidyl)benzimidazol-1-yl]piperidine-2,6-dione (820 mg, 1.80 mmol, TFA, Intermediate AZK) in THF (5 mL) was added TEA (181 mg, 1.80 mmol, 250 uL), and the mixture was stirred at −10° C. for 10 mins. Then tert-butyl N-(4-formylcyclohexyl)carbamate (408 mg, 1.80 mmol, CAS #181308-57-6) and AcOH (107 mg, 1.80 mmol, 102 uL) was added to the mixture, and the mixture was stirred at −10° C. for 20 mins. Next, NaBH(OAc)₃ (495 mg, 2.34 mmol) was added to the mixture, and the mixture was stirred at −10° C. for 1 hr. On completion, the reaction mixture was quenched with H₂O (0.5 mL) and concentrated in vacuo to give a residue. The residue was purified by prep-HPLC (column: Phenomenex luna C18 250*50 mm*15 um; mobile phase: [water(FA)-ACN]; B %: 15%-45%, 20 min) to give the title compound (500 mg, 50% yield) as an-off white solid. ¹H NMR (400 MHz, DMSO-d₆) δ 11.09 (s, 1H), 7.03-6.99 (m, 2H), 6.99-6.93 (m, 1H), 6.69 (d, J=8.0 Hz, 1H), 5.36 (dd, J=5.2, 12.4 Hz, 1H), 2.96 (d, J=10.8 Hz, 3H), 2.92-2.83 (m, 1H), 2.76-2.62 (m, 2H), 2.24-2.04 (m, 5H), 2.03-1.93 (m, 1H), 1.85-1.70 (m, 9H), 1.45-1.35 (m, 11H), 1.20-0.79 (m, 5H). LC-MS (ESI⁺) m/z 554.5 (M+H)⁺.

### Step 2—3-[4-[1-[(4-Aminocyclohexyl)methyl]-4-piperidyl]-3-methyl-2-oxo-benzimidazol-1-yl] piperidine-2,6-dione

To a solution of tert-butyl N-[4-[[4-[1-(2,6-dioxo-3-piperidyl)-3-methyl-2-oxo-benzimidazol-4-yl]-1-piperidyl]methyl]cyclohexyl]carbamate (25.0 mg, 45.1 umol) in DCM (1 mL) was added TFA (308 mg, 2.70 mmol, 0.20 mL). Then, the reaction mixture was stirred at 25° C. for 2 hrs. On completion, the reaction mixture was concentrated in vacuo to give the title compound (25.0 mg, 98% yield, TFA salt) as colorless oil. LC-MS (ESI⁺) m/z 454.3 (M+H)⁺.

### 8-cyano-4-(isopropylamino)-[1,4]benzodioxino[2,3-b]pyridine-3-carboxylic acid (Intermediate CEZ)

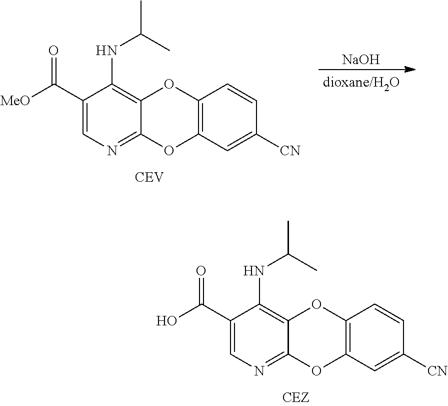 [SMILES: COC(=O)c1cnc2c(c1NC(C)C)Oc1ccc(C#N)cc1O2 ; "NaOH" ; CC(C)Nc1c(C(=O)O)cnc2c1Oc1ccc(C#N)cc1O2]

To a solution of methyl 8-cyano-4-(isopropylamino)-[1,4]benzodioxino[2,3-b]pyridine-3-carboxylate (50 mg, 153 umol, Intermediate CEV) in dioxane (1 mL) and H₂O (0.5 mL) was added NaOH (24.5 mg, 614 umol), then the mixture was stirred at 25° C. for 2 hrs. On completion, the reaction mixture was adjusted to pH=4, then extracted with DCM/MeOH=10/1 (5 mL×5). The organic layers were dried with Na₂SO₄, filtered and concentrated in vacuo to give the title compound (42 mg, 87% yield) as white solid. ¹H NMR (400 MHz, DMSO-d₆) δ 8.30 (d, J=8.0 Hz, 1H), 8.22 (s, 1H), 7.63 (d, J=2.0 Hz, 1H), 7.53 (dd, J=2.0, 8.4 Hz, 1H), 7.22 (d, J=8.4 Hz, 1H), 3.57 (s, 2H), 1.23 (d, J=6.4 Hz, 6H); LC-MS (ESI+) m/z 312.0 (M+H)⁺.

### 1-[8-[1-[(4-Aminocyclohexyl)methyl]-4-piperidyl]-4-isoquinolyl]hexahydropyrimidine-2,4-dione (Intermediate CFA)

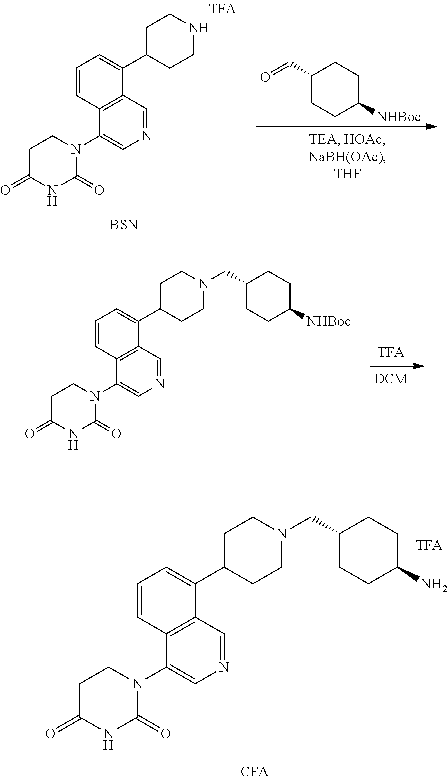

### Step 1—Tert-butyl N-[4-[[4-[4-(2,4-dioxohexahydropyrimidin-1-yl)-8-isoquinolyl]-1-piperidyl]methyl]cyclohexyl]carbamate

To a mixture of 1-[8-(4-piperidyl)-4-isoquinolyl]hexahydropyrimidine-2,4-dione (60.0 mg, 184 umol, Intermediate BSN) in THF (1 mL) was added TEA (18.7 mg, 184 umol) at −15° C. until the pH stabilized at 8. The mixture was stirred at −15° C. for 0.25 hr, then AcOH (11.1 mg, 184 umol) was added at −15° C. until the pH stabilized at 5-6. Subsequently, tert-butyl N-(4-formlylcyclohexyl)carbamate (33.6 mg, 147 umol) in DMF (1 mL) was added at −15° C. and the mixture was stirred for 0.25 hr. Next, NaBH(OAc)₃ (78.4 mg, 369 umol) was added in one portion. The resulting reaction mixture was stirred at −15° C. for 1 hr. On completion, the reaction mixture was concentrated in vacuo. The residue was purified by reversed phase flash (Phenomenex luna C18 150*25 mm*10 um; mobile phase: [water(FA)-ACN]; B %: 5%-35%, 10.5 min), to give the title compound (70.0 mg, 69% yield) as a white solid. LC-MS (ESI+) m/z 536.3 (M+H)⁺.

### Step 2—1-[8-[1-[(4-Aminocyclohexyl)methyl]-4-piperidyl]-4-isoquinolyl]hexahydropyrimidine-2,4-dione

To a solution of tert-butyl N-[4-[[4-[4-(2,4-dioxohexahydropyrimidin-1-yl)-8-isoquinolyl]-1-piperidyl]methyl]cyclohexyl]carbamate (70.0 mg, 130 umol) in DCM (2 mL) was added TFA (308 mg, 2.70 mmol). The mixture was then stirred at 25° C. for 1 hr. On completion, the reaction mixture was concentrated in vacuo to give the title compound (70.0 mg, 97% yield, TFA) as a yellow oil. LC-MS (ESI⁺) m/z 436.3 (M+H)⁺.

### 1-(5-Fluoro-8-piperazin-1-yl-4-isoquinolyl)hexahydropyrimidine-2,4-dione (Intermediate CFB)

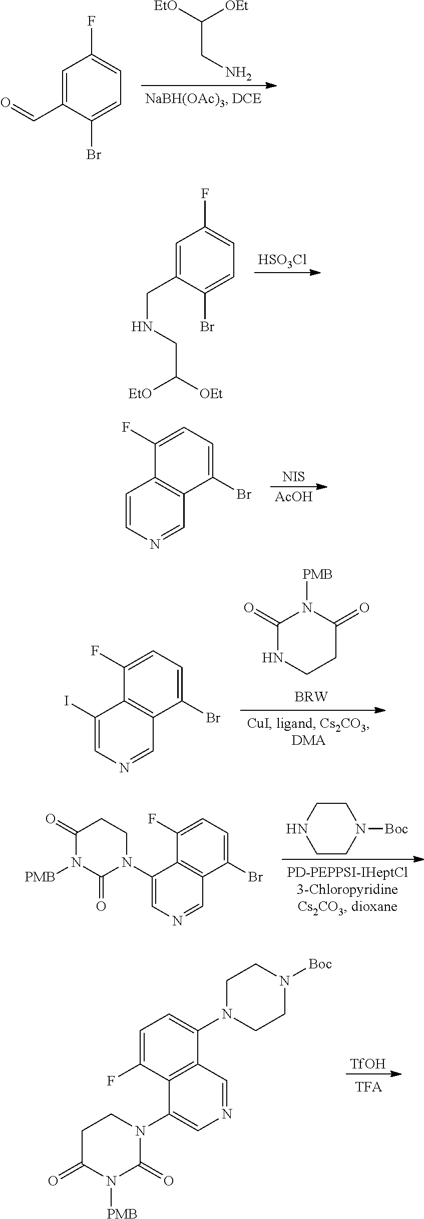

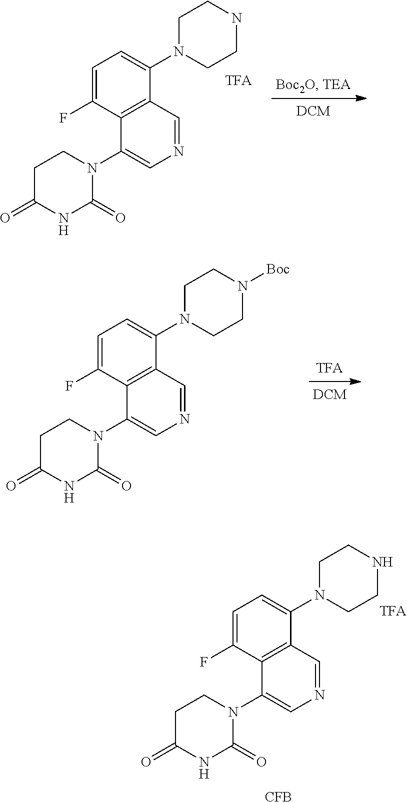

### Step 1—N-[(2-bromo-5-fluoro-phenyl)methyl]-2,2-diethoxy-ethanamine

To a solution of 2-bromo-5-fluoro-benzaldehyde (5.00 g, 24.6 mmol, CAS #59142-68-6) and 2,2-diethoxyethanamine (3.28 g, 24.6 mmol, CAS #645-36-3) in DCE (120 mL) was added NaBH(OAc)₃ (7.31 g, 34.4 mmol). The mixture was then stirred at 25° C. for 16 hrs. On completion, the mixture was diluted with DCM (100 mL) and quenched with NaOH aqueous (1M) until the pH=14. The organic layer was separated, washed with NaCl (100 mL), dried over Na₂SO₄, filtered and concentrated under reduced pressure to give the title compound (9.00 g, 79% yield) as yellow oil. LC-MS (ESI+) m/z 319.9 (M+H)⁺.

### Step 2—8-Bromo-5-fluoro-isoquinoline

To a solution of N-[(2-bromo-5-fluoro-phenyl)methyl]-2,2-diethoxy-ethanamine (4.91 g, 15.3 mmol) in sulfurochloridic acid (17.8 g, 153 mmol) was stirred at 100° C. for 0.5 hr. On completion, the mixture was quenched with ice and extracted with DCM (50 mL). The organic layer was discarded and the aqueous layer was basified with 50% NaOH solution until the pH=14, and then extracted with CH₂Cl₂ (50 mL×2). The combined organic layers were washed with brine, dried over Na₂SO₄, filtered and concentrated in vacuo. The residue was purified by column chromatography (SiO₂, Petroleum ether/Ethyl acetate=20:1 to 10:1) to give the title compound (1.50 g, 37% yield) as yellow solid. LC-MS (ESI+) m/z 227.9 (M+H)⁺.

### Step 3—8-Bromo-5-fluoro-4-iodo-isoquinoline

To a solution of 8-bromo-5-fluoro-isoquinoline (0.9 g, 3.98 mmol) in CH₃COOH (10 mL) was added NIS (1.34 g, 5.97 mmol). The mixture was stirred at 80° C. for 16 hrs. On completion, the reaction mixture was quenched with Na₂S₂O₄ (0.1 mL). The mixture was quenched with ice and extracted with DCM (10 mL), and the mixture was concentrated to give a residue. The residue was purified by column chromatography (SiO₂, Petroleum ether/Ethyl acetate=30/1 to 20/1) to give the title compound (876 mg, 60% yield) as yellow solid. LC-MS (ESI+) m/z 353.7 (M+H)⁺.

### Step 4—1-(8-Bromo-5-fluoro-4-isoquinolyl)-3-[(4-methoxyphenyl)methyl]hexahydropyrimidine-2,4-dione

A mixture of 8-bromo-5-fluoro-4-iodo-isoquinoline (500 mg, 1.42 mmol), 3-[(4-methoxyphenyl) methyl]hexahydropyrimidine-2,4-dione (624 mg, 2.66 mmol, Intermediate BRW), CuI (33.8 mg, 177 umol), Cs₂CO₃ (1.74 g, 5.33 mmol), 4 Å molecular sieves (50.0 mg, 1.78 mmol) and 3,4,7,8-tetramethyl-1,10-phenanthroline (41.9 mg, 177 umol) in DMA (5 mL) was degassed and purged with N₂ three times. Then the mixture was stirred at 95° C. for 5 hrs under N₂ atmosphere. On completion, the mixture was filtered and concentrated to give a residue. The crude product was purified by reversed-phase HPLC (0.10% FA condition) to give the title compound (130 mg, 14% yield) as yellow solid. LC-MS (ESI+) m/z 458 (M+H)⁺.

### Step 5—Tert-butyl 4-[5-fluoro-4-[3-[(4-methoxyphenyl)methyl]-2,4-dioxo-hexahydropyrimidin-1-yl]-8-isoquinolyl]piperazine-1-carboxylate

A mixture of 1-(8-bromo-5-fluoro-4-isoquinolyl)-3-[(4-methoxyphenyl)methyl]hexahydropyrimidine-2,4-dione (73.0 mg, 159 umol), tert-butyl piperazine-1-carboxylate (98.0 mg, 398 umol, HOAc, CAS #143238-38-4), 4 Å molecular sieves (25.0 mg, 199 umol), Cs₂CO₃ (194 mg, 597 umol) and 1,3-bis[2,6-bis(1-propylbutyl)phenyl]-4,5-dichloro-2H-imidazol-1-ium-2-ide; 3-chloropyridine; dichloropalladium (19.3 mg, 19.9 umol) in DMSO (2.5 mL) was degassed and purged with N₂ for 3 times, and then the mixture was stirred at 90° C. for 16 hrs under N₂ atmosphere. On completion, the reaction mixture was filtered and concentrated in vacuo to give a residue. The residue was then diluted with H₂O (8 mL) and extracted with EA (10 mL×3). The mixture was filtered and organic layers were dried over Na₂SO₄, filtered and concentrated in vacuo to give a residue. The residue was purified by column chromatography (Petroleum ether:Ethyl acetate=5:1 to 1:1) to give the title compound (120 mg, 86% yield) as black oil. LC-MS (ESI+) m/z 564.2 (M+H)⁺.

### Step 6-1-(5-Fluoro-8-piperazin-1-yl-4-isoquinolyl)hexahydropyrimidine-2,4-dione

To a solution of tert-butyl 4-[5-fluoro-4-[3-[(4-methoxyphenyl)methyl]-2,4-dioxo-hexahydropyrimidin-1-yl]-8-isoquinolyl]piperazine-1-carboxylate (100 mg, 177 umol) was added TfOH (0.1 mL) and TFA (1 mL). The mixture was stirred at 70° C. for 16 hrs. On completion, the mixture was concentrated to give the title compound (60.0 mg, 68% yield) as black oil. LC-MS (ESI+) m/z 344.0 (M+H)⁺.

### Step 7—Tert-butyl 4-[4-(2,4-dioxohexahydropyrimidin-1-yl)-5-fluoro-8-isoquinolyl]piperazine-1-carboxylate

To a solution of 1-(5-fluoro-8-piperazin-1-yl-4-isoquinolyl)hexahydropyrimidine-2,4-dione (50.0 mg, 145 umol) in DCM (0.5 mL) was added TEA (14.7 mg, 145 umol) at 0° C. until the pH stabilized at 9-10. Then to the mixture was added Boc₂O (34.9 mg, 160 umol) in DCM (0.5 mL) at 0° C. The mixture was stirred at 25° C. for 3 hrs. On completion, the mixture was concentrated in vacuo to give the title compound (60 mg, 92% yield) as red oil. LC-MS (ESI+) m/z 444.0 (M+H)⁺.

### Step 8—1-(5-Fluoro-8-piperazin-1-yl-4-isoquinolyl)hexahydropyrimidine-2,4-dione

To a solution of 1-(5-fluoro-8-piperazin-1-yl-4-isoquinolyl)hexahydropyrimidine-2,4-dione (50.0 mg, 145 umol) in DCM (0.5 mL) was added TEA (14.7 mg, 145 umol) at 0° C. until the pH stabilized at 9-10. Then the mixture was added Boc₂O (34.9 mg, 160 umol) in DCM (0.5 mL) at 0° C. The mixture was stirred at 25° C. for 3 hrs. On completion, the mixture was concentrated to give the title compound (25.0 mg, 53% yield) as red oil. LC-MS (ESI+) m/z 344.1 (M+H)⁺.

### 1-(4-Piperazin-1-ylphenyl)hexahydropyrimidine-2,4-dione (Intermediate CFC)

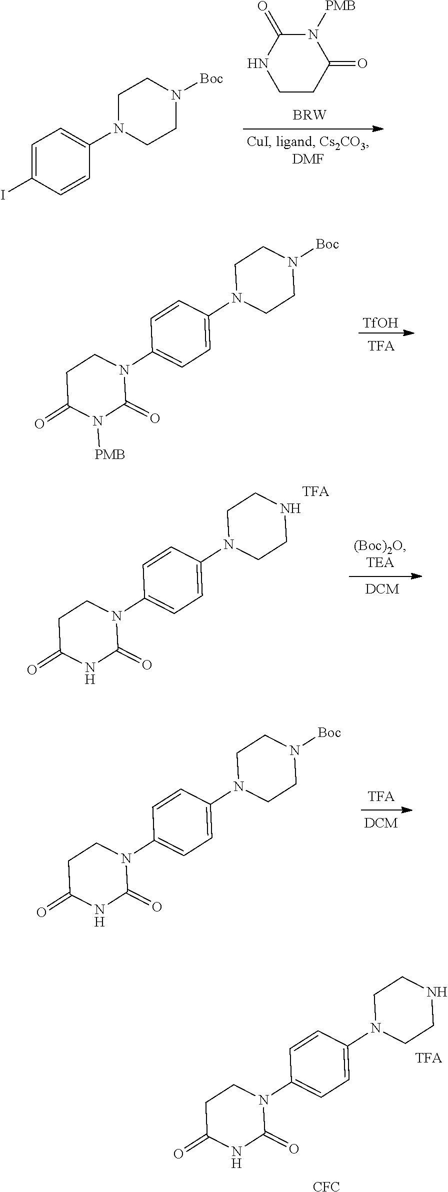

### Step 1—Tert-butyl 4-[4-[3-[(4-methoxyphenyl)methyl]-2,4-dioxo-hexahydropyrimidin-1-yl]phenyl] piperazine-1-carboxylate

A mixture of tert-butyl 4-(4-iodophenyl)piperazine-1-carboxylate (500 mg, 1.29 mmol, CAS #151978-66-4), 3-[(4-methoxyphenyl)methyl]hexahydropyrimidine-2,4-dione (331 mg, 1.42 mmol, Intermediate BRW), Cs₂CO₃ (839 mg, 2.58 mmol), CuI (49.0 mg, 257 umol) and (1R,2R)-N1,N2-dimethylcyclohexane-1,2-diamine (36.6 mg, 257 umol) in DMF (8 mL) was degassed and purged with N₂ three times. Then the mixture was stirred at 70° C. for 12 hr under N₂ atmosphere. On completion, the reaction mixture was filtered and concentrated in vacuo. The residue was purified by column chromatography (SiO₂, Petroleum ether/Ethyl acetate=50/1 to 10/1) to give the title compound (630 mg, 98% yield) as brown solid. LC-MS (ESI⁺) m/z 495.1 (M+H)⁺.

### Step 2—1-(4-Piperazin-1-ylphenyl)hexahydropyrimidine-2,4-dione

Tert-butyl 4-[4-[3-[(4-methoxyphenyl)methyl]-2,4-dioxo-hexahydropyrimidin-1-yl]phenyl]piperazine-1-carboxylate (320 mg, 647 umol) was dissolved in TFA (2 mL) and TfOH (0.4 mL). The mixture was stirred at 70° C. for 12 hr. On completion, the reaction mixture was concentrated in vacuo to give the title compound (250 mg, 99% yield, TFA) as black brown oil. LC-MS (ESI⁺) m/z 274.9 (M+H)⁺.

### Step 3—Tert-butyl 4-[4-(2,4-dioxohexahydropyrimidin-1-yl)phenyl]piperazine-1-carboxylate

To a solution of 1-(4-piperazin-1-ylphenyl)hexahydropyrimidine-2,4-dione (250 mg, 643 umol, TFA) in DCM (5 mL) was added TEA (130 mg, 1.29 mmol) at 25° C. until the pH stabilized at 8. Then Boc₂O (281 mg, 1.29 mmol) was added and the mixture was stirred at 25° C. for 4 hrs. On completion, the reaction mixture was quenched with H₂O (10 mL) at 25° C., and then extracted with EA 40 mL (20 mL×2). The combined organic layers were filtered and concentrated under reduced pressure to give a residue. The residue was purified by reversed phase (0.1% FA condition) to give the title compound (160 mg, 66% yield) as pink solid. ¹H NMR (400 MHz, DMSO-d₆) δ 10.28 (s, 1H), 7.17 (d, J=8.8 Hz, 2H), 6.95 (d, J=8.8 Hz, 2H), 3.70 (t, J=6.4 Hz, 2H), 3.45 (d, J=4.8 Hz, 4H), 3.13-3.03 (m, 4H), 2.68 (t, J=6.8 Hz, 2H), 1.42 (s, 9H); LC-MS (ESI⁺) m/z 375.1 (M+H)⁺.

### Step 4—1-(4-Piperazin-1-ylphenyl)hexahydropyrimidine-2,4-dione

To a solution of tert-butyl 4-[4-(2,4-dioxohexahydropyrimidin-1-yl)phenyl]piperazine-1-carboxylate (60.0 mg, 160 umol) in DCM (1 mL) was added TFA (182 mg, 1.60 mmol). The mixture was stirred at 25° C. for 1 hr. On completion, the reaction mixture was concentrated in vacuo to give the title compound (60.0 mg, 96% yield, TFA) as brown solid. LC-MS (ESI⁺) m/z 274.8 (M+H)⁺.

### 1-(6-Piperazin-1-yl-3-pyridyl)hexahydropyrimidine-2,4-dione (Intermediate CFD)

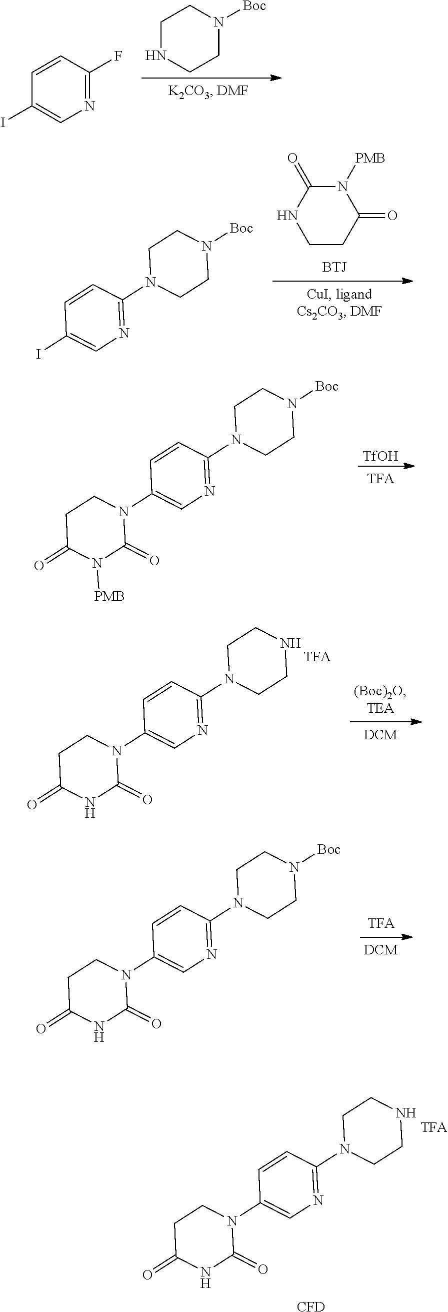

### Step 1—Tert-butyl 4-(5-iodo-2-pyridyl)piperazine-1-carboxylate

To a solution of 2-fluoro-5-iodo-pyridine (3.00 g, 13.4 mmol, CAS #171193-80-1) and tert-butyl piperazine-1-carboxylate (3.98 g, 16.1 mmol, HOAc, CAS #143238-38-4) in DMF (30 mL) was added K₂CO₃ (3.72 g, 26.9 mmol). The mixture was then stirred at 110° C. for 12 hrs. On completion, the mixture was quenched with water (120 mL) and extracted with EA (50 mL×3). The combined organic phase was dried over anhydrous sodium sulfate, filtered and concentrated to give a residue. The residue was purified by column chromatography (SiO₂, PE:EA=10:1 to 7:1), to give the title compound (4.22 g, 79% yield) as a white solid. LC-MS (ESI+) m/z 389.9 (M+H)⁺.

### Step 2—Tert-butyl 4-[5-[3-[(4-methoxyphenyl)methyl]-2,4-dioxo-hexahydropyrimidin-1-1-yl]-2-pyridyl]piperazine-1-carboxylate

To a solution of 3-[(4-methoxyphenyl)methyl]hexahydropyrimidine-2,4-dione (406 mg, 1.73 mmol, Intermediate BTJ) in DMF (7 mL) was added tert-butyl 4-(5-iodo-2-pyridyl)piperazine-1-carboxylate (450 mg, 1.16 mmol), CuI (88.0 mg, 462 umol,), Cs₂CO₃ (753 mg, 2.31 mmol), (1R,2R)-N1,N2-dimethylcyclohexane-1,2-diamine (65.7 mg, 462 umol) and 4 Å molecular sieves (100 mg, 1.16 mmol) under N₂. On completion, the mixture was stirred at 70° C. for 12 hrs. On completion, the mixture was filtered and concentrated in vacuo to give a residue. The residue was purified by column chromatography (SiO₂, Petroleum ether/Ethyl acetate=3:1 to 1:1) to give the title compound (600 mg, 74% yield) as a white solid. LC-MS (ESI+) m/z 496.2 (M+H)⁺.

### Step 3—1-(6-Piperazin-1-yl-3-pyridyl)hexahydropyrimidine-2,4-dione

To a solution of tert-butyl 4-[5-[3-[(4-methoxyphenyl)methyl]-2,4-dioxo-hexahydropyrimidin-1-yl]-2-pyridyl]piperazine-1-carboxylate (400 mg, 807 umol) in TFA (2 mL) was added TfOH (0.2 mL). The mixture was stirred at 70° C. for 2 hrs. On completion, the reaction mixture was concentrated in vacuo to give the title compound (310 mg, 98% yield, TFA) as a yellow oil. LC-MS (ESI+) m/z 276.0 (M+H)⁺.

### Step 4—Tert-butyl 4-[5-(2,4-dioxohexahydropyrimidin-1-yl)-2-pyridyl]piperazine-1-carboxylate

To a solution of 1-(6-piperazin-1-yl-3-pyridyl)hexahydropyrimidine-2,4-dione (340 mg, 873 umol, TFA) in DCM (1 mL) was added TEA (88.3 mg, 873 umol) at 0° C. until pH stabilized at 9-10. Then the mixture was added (Boc)₂O (209 mg, 960 umol) in DCM (0.5 mL) at 0° C. The mixture was stirred at 25° C. for 3 hrs. On completion, the mixture was quenched with water (5 mL) and extracted with ethyl acetate (10 mL×3). The combined organic phase was dried over anhydrous sodium sulfate, filtered and concentrated to give a residue. The crude product was triturated with PE:DCM=7:1 (10 mL) at 25° C. for 10 mins to give the title compound (320 mg, 96% yield) as a yellow solid. LC-MS (ESI+) m/z 376.1 (M+H)⁺.

### Step 5—1-(6-Piperazin-1-yl-3-pyridyl)hexahydropyrimidine-2,4-dione

To a solution of tert-butyl 4-[5-(2,4-dioxohexahydropyrimidin-1-yl)-2-pyridyl]piperazine-1-carboxylate (80.0 mg, 213 umol) in DCM (1 mL) was added TFA (154 mg, 1.35 mmol). The mixture was stirred at 25° C. for 1 hr. On completion, the reaction mixture was concentrated in vacuo to give the title compound (80.0 mg, 96% yield, TFA) as a yellow oil. LC-MS (ESI+) m/z 276.1 (M+H)⁺.

### 2-(2,6-dioxo-3-piperidyl)-4-[2-(methylamino)ethylamino]isoindoline-1,3-dione (Intermediate CFE)

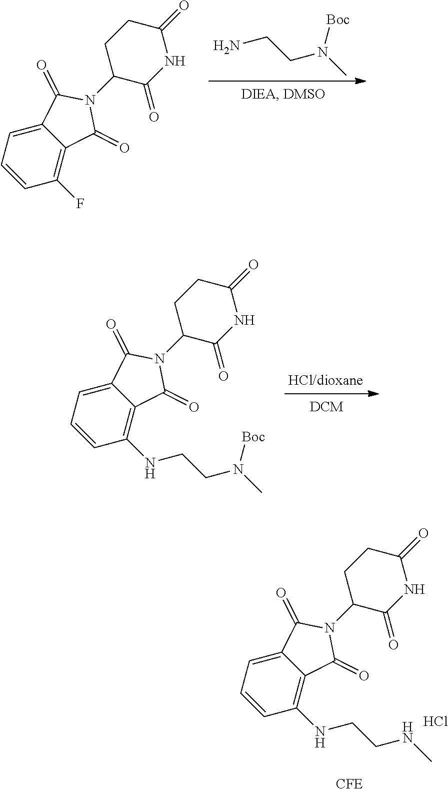 [SMILES: O=C1CCC(N2C(=O)c3cccc(F)c3C2=O)C(=O)N1 ; CN(CCN)C(=O)OC(C)(C)C ; CN(CCNc1cccc2c1C(=O)N(C1CCC(=O)NC1=O)C2=O)C(=O)OC(C)(C)C ; CNCCNc1cccc2c1C(=O)N(C1CCC(=O)NC1=O)C2=O ; Cl]

### Step 1—Tert-butyl N-[2-[[2-(2,6-dioxo-3-piperidyl)-1,3-dioxo-isoindolin-4-yl]amino]ethyl]-N-methyl-carbamate

To a solution of 2-(2,6-dioxo-3-piperidyl)-4-fluoro-isoindoline-1,3-dione (1.00 g, 3.62 mmol, CAS #835616-60-9) and tert-butyl N-(2-aminoethyl)-N-methyl-carbamate (756 mg, 4.34 mmol, CAS #121492-06-6) in DMSO (8 mL) was added DIEA (1.40 g, 10.8 mmol, 1.89 mL). Then the reaction mixture was stirred at 130° C. for 4 hrs. On completion, the reaction mixture was added to water (40 ml) dropwise, then filtered and the filter cake was concentrated in vacuo to give the title compound (1.5 g, 96% yield) as a green solid. LC-MS (ESI⁺) m/z 330.9 (M−100+H)⁺.

### Step 2—2-(2,6-Dioxo-3-piperidyl)-4-[2-(methylamino)ethylamino]isoindoline-1,3-dione

To a solution of tert-butyl N-[2-[[2-(2,6-dioxo-3-piperidyl)-1,3-dioxo-isoindolin-4-yl]amino]ethyl]-N-methyl-carbamate (100 mg, 232 umol) in DCM (1 mL) was added HCl/dioxane (4 M, 1 mL), then the reaction mixture was stirred at 25° C. for 1 hr. On completion, the reaction mixture was concentrated in vacuo to give the title compound (85 mg, 99% yield, HCl) as blue oil. LC-MS (ESI⁺) m/z 330.8 (M+H)⁺.

### 4-[5-Methoxy-6-[[6-(trifluoromethyl) pyridine-2-carbonyl]amino]-1,3-benzothiazol-2-yl]cyclohexanecarboxylic acid (Intermediate CFF)

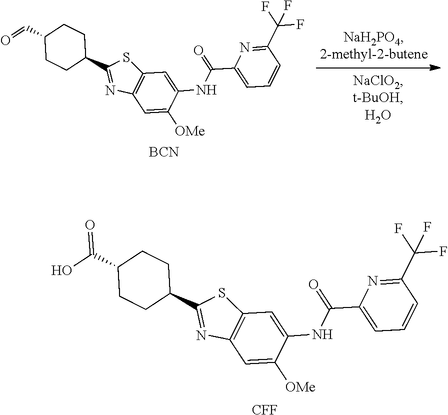

To a mixture of N-[2-(4-formylcyclohexyl)-5-methoxy-1,3-benzothiazol-6-yl]-6-(trifluoromethyl)pyridine-2-carboxamide (100 mg, 215 umol, Intermediate BCN) in t-BuOH (3 mL) and H₂O (1 mL) was added 2-methylbut-2-ene (60.5 mg, 863 umol, 91.4 uL), NaH₂PO₄ (25.8 mg, 215 umol) and sodium chlorite (58.5 mg, 647 umol) at 0° C. Then the reaction mixture was stirred at 25° C. for 2 hours. On completion, the residue was diluted with water (10 mL), then the residue was extracted with DCM (3×30 mL). The combined organic layer was dried over Na₂SO₄, filtered and concentrated in vacuo to give the title compound (100 mg, 97% yield) as yellow solid. LC-MS (ESI⁺) m/z 480.0 (M+H)⁺.

### N-[2-(4-formylcyclohexyl)-6-methoxy-indazol-5-yl]-5-methyl-pyridine-3-carboxamide (Intermediate CFG)

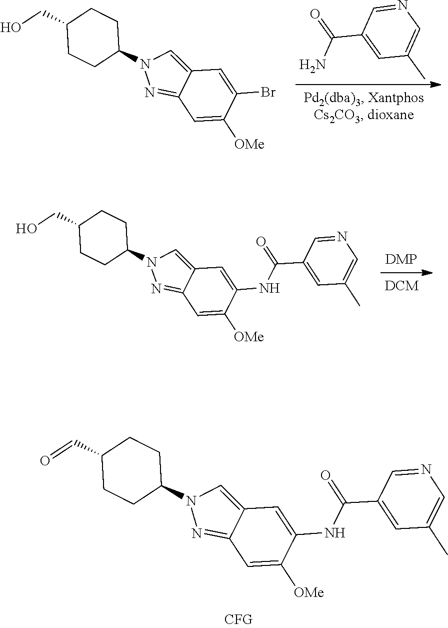

### Step 1—N-[2-[4-(hydroxymethyl)cyclohexyl]-6-methoxy-indazol-5-yl]-5-methyl-pyridine-3-carboxamide

To a mixture of [4-(5-bromo-6-methoxy-indazol-2-yl)cyclohexyl]methanol (500 mg, 1.47 mmol, synthesized via Steps 1-3 of Intermediate ATE) and 5-methylpyridine-3-carboxamide (240 mg, 1.77 mmol, CAS #70-57-5) in dioxane (10 mL) was added Pd₂(dba)₃ (134 mg, 147 umol), Xantphos (85.2 mg, 147 umol) and Cs₂CO₃ (1.44 g, 4.42 mmol). The reaction mixture was then stirred at 100° C. for 12 hours. On completion, the residue was diluted with water (30 mL) and extracted with DCM(2×30 mL). The combined organic layer was dried over Na₂SO₄, filtered and concentrated in vacuo to give a residue. The residue was purified by reverse phase (0.1% FA condition) to give the title compound (100 mg, 17% yield) as white solid. LC-MS (ESI⁺) m/z 395.1 (M+H)⁺.

### Step 2—N-[2-(4-formylcyclohexyl)-6-methoxy-indazol-5-yl]-5-methyl-pyridine-3-carboxamide

To a mixture of N-[2-[4-(hydroxymethyl)cyclohexyl]-6-methoxy-indazol-5-yl]-5-methyl-pyridine-3-carboxamide (100 mg, 253 umol) in DCM (5 mL) was added DMP (139 mg, 329 umol). The reaction mixture was stirred at 25° C. for 2 hours. On completion, the reaction mixture was quenched with saturated Na₂S₂O₃ (10 mL) and saturated NaHCO₃ (10 mL) at 25° C., then the mixture was stirred for 30 minutes. The residue was diluted with water (20 mL) and extracted with DCM (2×20 mL). The combined organic layer was dried over Na₂SO₄, filtered and concentrated in vacuo to give a residue to give the title compound (91 mg, 91% yield) as yellow solid. LC-MS (ESI⁺) m/z 393.2 (M+H)⁺.

### 3-[5-(2,5-Dihydro-1H-pyrrol-3-yl)-3-methyl-2-oxo-benzimidazol-1-yl]piperidine-2,6-dione (Intermediate CFH)

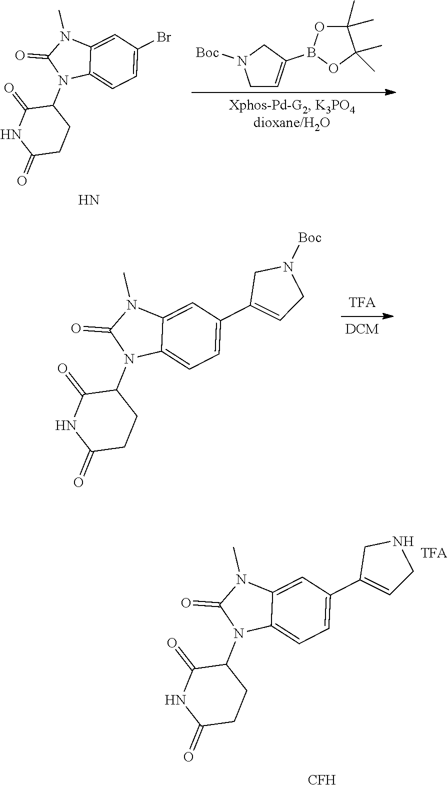

### Step 1—Tert-butyl3-(1-(2,6-dioxopiperidin-3-yl)-3-methyl-2-oxo-2,3-dihydro-1H-benzo[d]imidazol-5-yl)-2,5-dihydro-1H-pyrrole-1-carboxylate

A mixture of 3-(5-bromo-3-methyl-2-oxo-benzimidazol-1-yl)piperidine-2,6-dione (300 mg, 887 umol, Intermediate HN) tert-butyl 3-(4,4,5,5-tetramethyl-1,3,2-dioxaborolan-2-yl)-2,5-dihydropyrrole-1-carboxylate (340 mg, 1.15 mmol, CAS #212127-83-8), XPHOS-PDG2 (69.8 mg, 88.7 umol) and K₃PO₄ (564 mg, 2.66 mmol) in dioxane (5 mL) and H₂O (1 mL) was degassed and purged with N₂ three times. Then the mixture was stirred at 80° C. for 16 h under N₂ atmosphere. On completion, the reaction mixture was cooled to room temperature and filtered, the filtrate was concentrated in vacuo. The residue was purified by reverse phase (0.1% FA condition) to give the title compound (200 mg, 52% yield) as a white solid. LC-MS (ESI+) m/z 427.2 (M+H)⁺.

### 3-[5-(2,5-Dihydro-1H-pyrrol-3-yl)-3-methyl-2-oxo-benzimidazol-1-yl]piperidine-2,6-dione

To a solution of tert-butyl 3-[1-(2,6-dioxo-3-piperidyl)-3-methyl-2-oxo-benzimidazol-5-yl]-2,5-dihydropyrrole-1-carboxylate(60.0 mg, 140 umol) in DCM (2 mL) was added TFA (160 mg, 1.41 mmol). The mixture was stirred at 25° C. for 1 hr. On completion, the mixture was concentrated in vacuo to give the title compound (60.0 mg, 96% yield, TFA salt) as a yellow oil. LC-MS (ESI+) m/z 327.0 (M+H)⁺.

### N-(4-formylcyclohexyl)-4-(isopropylamino)-6-[5-(trifluoromethyl)pyrrolo[2,3-b]pyridin-1-yl]pyridine-3-carboxamide (Intermediate CFI)

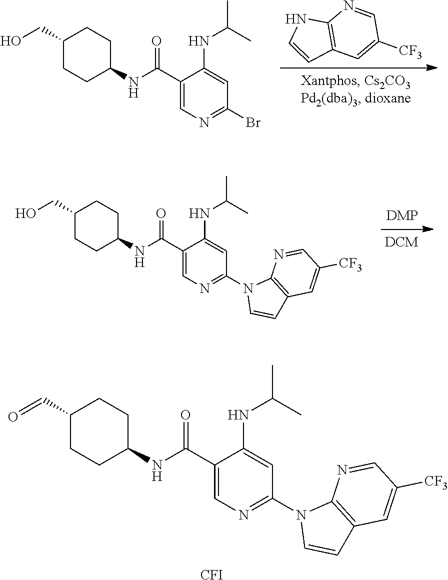 [SMILES: CC(C)Nc1cc(Br)ncc1C(=O)N[C@H]1CC[C@H](CO)CC1 ; FC(F)(F)c1cnc2[nH]ccc2c1 ; CC(C)Nc1cc(-n2ccc3cc(C(F)(F)F)cnc32)ncc1C(=O)N[C@H]1CC[C@H](CO)CC1 ; CC(C)Nc1cc(-n2ccc3cc(C(F)(F)F)cnc32)ncc1C(=O)N[C@H]1CC[C@H](C=O)CC1 ; "CFI"]

### Step 1—N-[4-(hydroxymethyl)cyclohexyl]-4-(isopropylamino)-6-[5-(trifluoromethyl)pyrrolo[2,3-b]pyridin-1-yl]pyridine-3-carboxamide

To a solution of 6-bromo-N-[4-(hydroxymethyl)cyclohexyl]-4-(isopropylamino)pyridine-3-carboxamide (50.0 mg, 135 umol, synthesized via Steps 1-4 of Intermediate BVU) and 5-(trifluoromethyl)-1H-pyrrolo [2,3-b]pyridine (27.7 mg, 148 umol, CAS #1036027-54-9) in dioxane (2 mL) was added TBUBRETTPHOS PD G3 (11.5 mg, 13.5 umol) and tBuONa (2 M, 203 uL)/ Then the mixture was stirred at 90° C. for 16 hours. On completion, the mixture was filtered and the filtrate was concentrated in vacuo to give the residue. The residue was purified by prep-HPLC (column: Phenomenex luna C18 150*25 mm*10 um; mobile phase: [water(FA)-ACN]; B %: 42%-72%, 10.5 min) to give the title compound (15.5 mg, 24% yield) as a yellow solid. ¹H NMR (400 MHz, DMSO-d₆) δ 8.77 (d, J=1.6 Hz, 1H), 8.62-8.51 (m, 4H), 8.30 (d, J=7.6 Hz, 1H), 8.18 (s, 1H), 6.90 (d, J=4.0 Hz, 1H), 4.41 (s, 1H), 3.83-3.66 (m, 2H), 3.24-3.22 (m, 2H), 2.52-2.51 (m, 1H), 1.91-1.84 (m, 2H), 1.80-1.77 (m, 2H), 1.34 (s, 2H), 1.30 (s, 3H), 1.29 (s, 3H), 1.03-0.92 (m, 2H). LC-MS (ESI⁺) m/z 476.5 (M+H)⁺.

### Step 2—N-(4-formylcyclohexyl)-4-(isopropylamino)-6-[5-(trifluoromethyl)pyrrolo[2,3-b]pyridin-1-yl]pyridine-3-carboxamide

To a solution of N-[4-(hydroxymethyl)cyclohexyl]-4-(isopropylamino)-6-[5-(trifluoromethyl)pyrrolo [2,3-b]pyridine-1-yl]pyridine-3-carboxamide (80.0 mg, 168 umol) in DCM (3 mL) was added DMP (78.1 u, 252 umol) at 0° C. Then the mixture was stirred at 25° C. for 1 hour. On completion, the mixture was quenched with sodium thiosulfate pentahydrate (10 mL) and NaHCO₃ (10 mL), then extracted with DCM (20 mL×3). The combined organic layer was dried over anhydrous Na₂SO₄, then concentrated in vacuo to give the title compound (74.0 mg, 93% yield) as a yellow solid. LC-MS (ESI+) m/z 474.3 (M+H)⁺.

### 1-(7-Bromo-8-methoxy-4-isoquinolyl)-3-[(4-methoxyphenyl) methyl]hexahydropyrimidine-2, 4-dione (Intermediate CFJ)

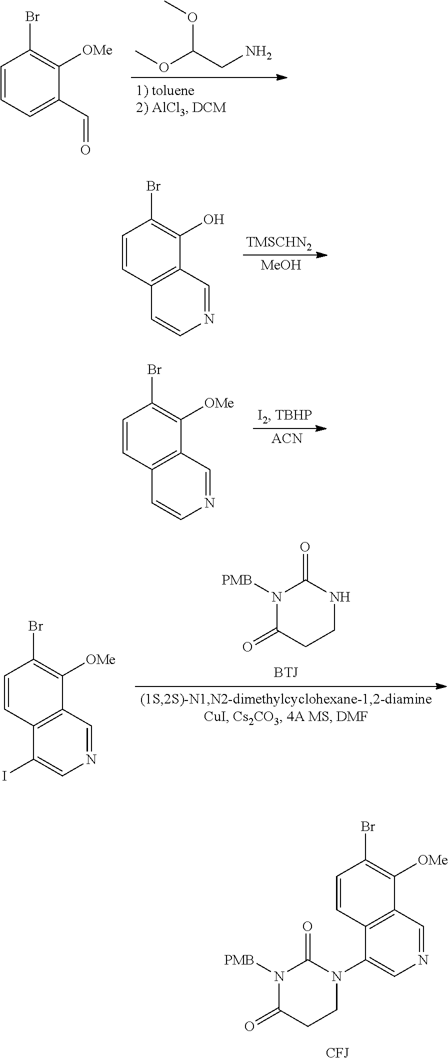

### Step 1—7-Bromoisoquinolin-8-ol

To a solution of 3-bromo-2-methoxy-benzaldehyde (3 g, 13.9 mmol, CAS #88275-87-0) in toluene (40 mL) was added 2,2-dimethoxyethanamine (1.54 g, 14.6 mmol, CAS #22483-09-6). The mixture was stirred at 100° C. for 4 hrs. Then the mixture was concentrated in vacuo and dissolved in DCM (20 mL) and cooled to 0° C. Next, AlCl₃ (6.14 g, 46.0 mmol) was added to above solution portion-wise and the resulting dark red suspension was left to stir at 0° C. for 30 minutes and was then allowed to slowly warm to 25° C. and stirred for 16 hours. On completion, the mixture was quenched with MeOH (20 mL) and concentrated in vacuo. The crude product was purified by prep-HPLC (revese phase: 0.1% FA) to give the title compound (1.2 g, 38% yield) as a yellow solid. ¹H NMR (400 MHz, DMSO-d₆) δ 10.61 (s, 1H), 9.56 (s, 1H), 8.50 (d, J=5.6 Hz, 1H), 7.83 (d, J=8.8 Hz, 1H), 7.77 (d, J=5.6 Hz, 1H), 7.39 (d, J=8.0 Hz, 1H). LC-MS (ESI⁺) m/z 224.0 (M+H)⁺.

### Step 2—7-Bromo-8-methoxy-isoquinoline

To a solution of 7-bromoisoquinolin-8-ol (1 g, 4.46 mmol) in MeOH (15 mL) was added TMSCHN₂ (2 M, 11.16 mL). The mixture was then stirred at 25° C. for 16 hrs. On completion, the mixture was quenched with HOAc (5 mL) and concentrated in vacuo. The crude product was purified by silica gel column to give the title compound (305 mg, 28% yield) as yellow solid. ¹H NMR (400 MHz, DMSO-d₆) δ 9.47 (s, 1H), 8.61 (d, J=6.0 Hz, 1H), 7.95 (d, J=8.8 Hz, 1H), 7.91-7.86 (m, 1H), 7.74 (d, J=8.8 Hz, 1H), 4.02 (s, 3H). LC-MS (ESI⁺) m/z 238.0 (M+H)⁺.

### Step 3—7-Bromo-4-iodo-8-methoxy-isoquinoline

To a solution of 7-bromo-8-methoxy-isoquinoline (305 mg, 1.28 mmol) in ACN (8 mL) was added I₂ (390 mg, 1.54 mmol) and TBHP (1.32 g, 10.2 mmol 70% solution). The mixture was stirred at 80° C. for 16 hrs. On completion, the mixture was quenched with sat. Na₂S₂O₃ (20 mL), then diluted with H₂O (50 mL), and extracted with EA (3×50 mL). The organic layers were washed with brine (3×50 mL), dried over anhydrous Na₂SO₄, filtered and concentrated in vacuo. The residue was purified by column chromatography (SiO₂, PE:EA=50:1) to give the title compound (429 mg, 90% yield) as yellow solid. ¹H NMR (400 MHz, DMSO-d₆) δ 9.40 (s, 1H), 9.02 (s, 1H), 8.11 (d, J=8.8 Hz, 1H), 7.71 (d, J=9.2 Hz, 1H), 4.03 (s, 3H). LC-MS (ESI⁺) m/z 365.8 (M+H)⁺.

### Step 4—1-(7-Bromo-8-methoxy-4-isoquinolyl)-3-[(4-methoxyphenyl) methyl]hexahydropyrimidine-2, 4-dione

To a solution of 7-bromo-4-iodo-8-methoxy-isoquinoline (300 mg, 824 umol) and 3-[(4-methoxyphenyl)methyl]hexahydropyrimidine-2,4-dione (212 mg, 906 umol, Intermediate BTJ) in DMF (20 mL) was added (1S,2S)-N1,N2-dimethylcyclohexane-1,2-diamine (46.9 mg, 329 umol), Cs₂CO₃ (537 mg, 1.65 mmol), 4 Å molecular sieves (50 mg) and CuI (62.7 mg, 329 umol). The mixture was degassed and purged with N₂ three times and the mixture was stirred at 70° C. for 16 hrs under N₂ atmosphere. On completion, the mixture was filtered and concentrated in vacuo. The mixture was purified by reversed phase (0.1% FA) to give the title compound (130 mg, 33% yield) as brown oil. ¹H NMR (400 MHz, DMSO-d₆) δ 9.46 (s, 1H), 8.67 (s, 1H), 7.97 (d, J=8.8 Hz, 1H), 7.70 (d, J=9.6 Hz, 1H), 7.25 (d, J=8.8 Hz, 2H), 6.90-6.86 (m, 2H), 4.83 (s, 2H), 4.04 (s, 3H), 3.73 (s, 3H), 3.20-3.06 (m, 2H), 3.04-2.71 (m, 2H). LC-MS (ESI⁺) m/z 472.1 (M+H)⁺.

### 1-(8-Methoxy-7-piperazin-1-yl-4-isoquinolyl)hexahydropyrimidine-2,4-dione (Intermediate CFK)

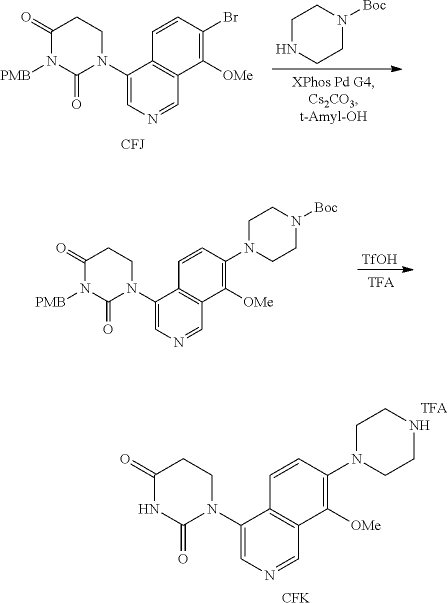

### Step 1—Tert-butyl 4-[8-methoxy-4-[3-[(4-methoxyphenyl)methyl]-2,4-dioxo-hexahydropyrimidin-1-yl]-7-isoquinolyl]piperazine-1-carboxylate

To a mixture of 1-(7-bromo-8-methoxy-4-isoquinolyl)-3-[(4-methoxyphenyl)methyl]hexahydropyrimidine-2,4-dione (250 mg, 531 umol, Intermediate CFJ) and tert-butyl piperazine-1-carboxylate (297 mg, 1.59 mmol, CAS #143238-38-4) in t-Amyl-OH (3 mL) was added Xphos Pd G4 (45.7 mg, 53.1 umol) and Cs₂CO₃ (519 mg, 1.59 mmol). The reaction mixture was stirred at 100° C. for 1 hour. On completion, the reaction mixture was filtered and diluted with water (10 mL) and extracted with EA (2×30 mL). The combined organic layer was dried over Na₂SO₄, filtered and concentrated in vacuo to give a residue. The residue was purified by reverse phase (0.1% FA condition) to give the title compound (80.0 mg, 26% yield) as white solid. ¹H NMR (400 MHz, DMSO-d₆) δ 9.37 (s, 1H), 8.42 (s, 1H), 7.65-7.56 (m, 2H), 7.25 (d, J=8.4 Hz, 2H), 6.88 (d, J=8.4 Hz, 2H), 4.82 (s, 2H), 3.99 (s, 3H), 3.94-3.86 (m, 1H), 3.76-3.69 (m, 4H), 3.56 (s, 4H), 3.19-3.13 (m, 4H), 3.13-3.05 (m, 1H), 3.00-2.92 (m, 1H), 1.43 (s, 9H).

### Step 2—1-(8-Methoxy-7-piperazin-1-yl-4-isoquinolyl)hexahydropyrimidine-2,4-dione

To a mixture of tert-butyl 4-[8-methoxy-4-[3-[(4-methoxyphenyl)methyl]-2,4-dioxo-hexahydropyrimidin-1-yl]-7-isoquinolyl]piperazine-1-carboxylate (80.0 mg, 138 umol) in TFA (3 mL) was added TfOH (850 mg, 5.66 mmol, 0.5 mL). The reaction mixture was then stirred at 80° C. for 1 hour. On completion, the reaction mixture was concentrated in vacuo to give the title compound (60.0 mg, 91% yield, TFA) as red oil. LC-MS (ESI+) m/z 356.1 (M+H)⁺.

### N-[6-ethyl-2-(4-formylcyclohexyl)indazol-5-yl]-6-(trifluoromethyl)pyridine-2-carboxamide (Intermediate CFL)

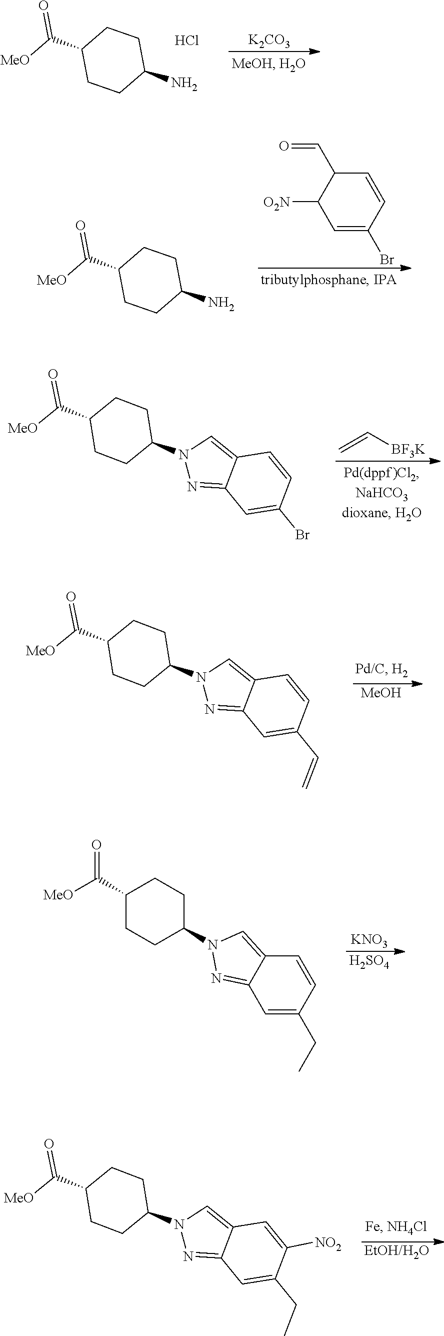

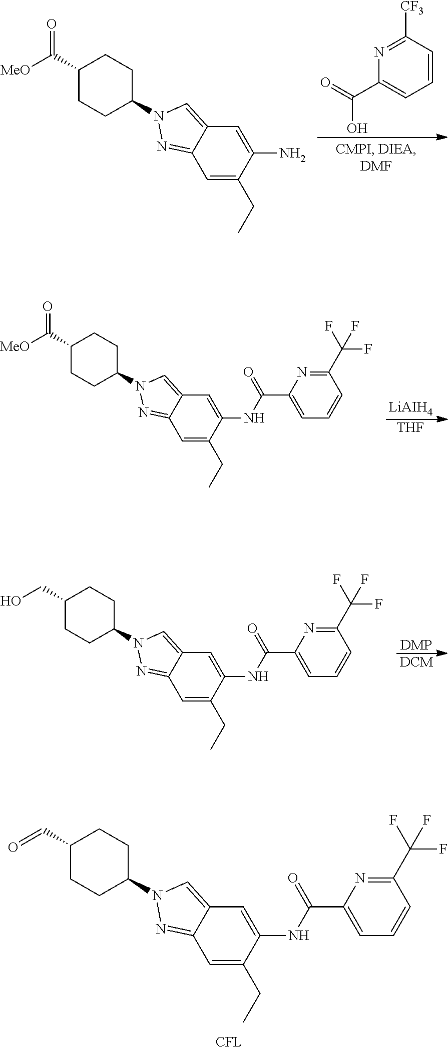

### Step 1—Methyl 4-aminocyclohexanecarboxylate

To a mixture of methyl 4-aminocyclohexanecarboxylate (5.00 g, 25.8 mmol, HCl, CAS #61367-07-5) in MeOH (50 mL) and H₂O (0.5 mL) was added K₂CO₃ (3.57 g, 25.8 mmol) at 25° C. The mixture was then stirred at 25° C. for 4 hrs. On completion, the reaction mixture was filtered and the filtrate concentrated in vacuo to give the title compound (4.00 g, 98% yield) as a white solid.

### Step 2—Methyl 4-(6-bromoindazol-2-yl)cyclohexanecarboxylate

To a solution of methyl 4-aminocyclohexanecarboxylate (4.00 g, 25.4 mmol) in IPA (50 mL) was added 4-bromo-2-nitro-benzaldehyde (5.85 g, 25.4 mmol, CAS #5551-12-2). The mixture was then stirred at 80° C. for 3 hours under N₂ atmosphere. Next, tributylphosphane (15.4 g, 76.3 mmol) was added at 25° C. then the reaction mixture was stirred at 80° C. for 12 hours under N₂ atmosphere. On completion, the mixture was concentrated in vacuo, then triturated with petroleum ether (100 mL) at 25° C. for 10 mins and filtered. The filter cake was dried in vacuo to give the title compound (6.00 g, 69% yield) as a yellow solid. LC-MS (ESI⁺) m/z 338.9 (M+3)+.

### Step 3—Methyl 4-(6-vinylindazol-2-yl)cyclohexanecarboxylate

A mixture of methyl 4-(6-bromoindazol-2-yl)cyclohexanecarboxylate (6.00 g, 17.7 mmol), potassium hydride; trifluoro(vinyl)boron (7.15 g, 53.3 mmol, CAS #233664-53-4), Pd(dppf)Cl₂ (650 mg, 889 umol) and NaHCO₃ (2.99 g, 35.5 mmol) in a mixed solvent of dioxane (100 mL) and H₂O (10 mL) was stirred at 90° C. for 4 hours under nitrogen atmosphere. On completion, the reaction mixture was filtered and the filtrate was concentrated in vacuo. The residue was purified by silica gel chromatography (petroleum ether:ethyl acetate=5:1) to give the title compound (4.00 g, 79% yield) as a brown solid. LC-MS (ESI⁺) m/z 285.1 (M+H)⁺.

### Step 4—Methyl 4-(6-ethylindazol-2-yl)cyclohexanecarboxylate

To a mixture of methyl 4-(6-vinylindazol-2-yl)cyclohexanecarboxylate (2.00 g, 7.0 mmol) in MeOH (15 mL) was added Pd/C (50.0 mg, 7.0 mmol, 10 wt %). The mixture was added at 25° C. for 0.5 hr under H₂ (15 Psi). On completion, the mixture was filtered and concentrated to give the title compound (1.90 g, 91% yield) as yellow solid. ¹H NMR (400 MHz, DMSO-d₆) δ 8.28 (s, 1H), 7.58 (d, J=8.4 Hz, 1H), 7.34 (s, 1H), 6.90 (d, J=8.4 Hz, 1H), 4.45 (m, 1H), 3.80-3.43 (m, 3H), 2.66 (q, J=7.6 Hz, 2H), 2.47-2.38 (m, 1H), 2.17-1.89 (m, 6H), 1.59 (m, 2H), 1.24-1.18 (m, 3H); LC-MS (ESI+) m/z 287.0 (M+H)⁺.

### Step 5—Methyl 4-(6-ethyl-5-nitro-indazol-2-yl)cyclohexanecarboxylate

To a solution of methyl 4-(6-ethylindazol-2-yl)cyclohexanecarboxylate (600 mg, 2.10 mmol) in H₂SO₄ (5 mL) was added KNO₃ (211 mg, 2.10 mmol) at −10° C. The reaction mixture was stirred at −10° C. for 3 hrs. On completion, the mixture was quenched with ice water and extracted with ethyl acetate (50 mL×3). The combined organic phase was washed with NaHCO₃ (80 mL) and H₂O (80 mL), dried over anhydrous sodium sulfate, filtered and concentrated to the title compound (680 mg, 94% yield) as yellow oil. ¹H NMR (400 MHz, DMSO-d₆) δ 8.68 (s, 1H), 8.52 (s, 1H), 7.62 (s, 1H), 4.61-4.53 (m, 1H), 3.63 (d, J=1.6 Hz, 3H), 2.90 (q, J=7.6 Hz, 2H), 2.46-2.39 (m, 1H), 2.16 (d, J=12.4 Hz, 2H), 2.07 (d, J=13.6 Hz, 2H), 1.97-1.90 (m, 2H), 1.59 (d, J=12.4 Hz, 2H), 1.21-1.17 (m, 3H); LC-MS (ESI+) m/z 331.9 (M+H)⁺.

### Step 6—Methyl 4-(5-amino-6-ethyl-indazol-2-yl)cyclohexanecarboxylate

A mixture of methyl 4-(6-ethyl-5-nitro-indazol-2-yl)cyclohexanecarboxylate (680 mg, 2.0 mmol), Fe (802 mg, 14.3 mmol) and NH₄Cl (1.10 g, 20.5 mmol) in a mixed solvent of EtOH (10 mL) and H₂O (2 mL) was stirred at 80° C. for 12 hrs under N₂. On completion, the reaction mixture was filtered through celite and the filtrate was concentrated in vacuo. The residue was purified by prep-HPLC purification (column: YMC Triart C18 250*50 mm*7 um; mobile phase: [water (FA)-ACN]; B %: 17%-47%, 10 min) to give the title compound (110 mg, 17% yield) as brown solid. ¹H NMR (400 MHz, DMSO-d₆) δ 7.88 (s, 1H), 7.18 (s, 1H), 6.65 (s, 1H), 4.75-4.42 (m, 2H), 4.41-4.24 (m, 1H), 3.62 (s, 3H), 2.59-2.54 (m, 2H), 2.46-2.38 (m, 1H), 2.13-2.01 (m, 4H), 1.97-1.83 (m, 2H), 1.63-1.50 (m, 2H), 1.19 (t, J=7.2 Hz, 3H); LC-MS (ESI+) m/z 301.9 (M+H)⁺.

### Step 7—Methyl 4-[6-ethyl-5-[[6-(trifluoromethyl)pyridine-2-carbonyl]amino]indazol-2-yl]cyclo hexanecarboxylate

A mixture of 6-(trifluoromethyl)pyridine-2-carboxylic acid (57.0 mg, 298.6 umol, CAS #131747-42-7), CMPI (99.1 mg, 388 umol) and DIPEA (115 mg, 895 umol) in DMF (0.5 mL) was stirred at 25° C. for 15 min. Then a solution of methyl 4-(5-amino-6-ethyl-indazol-2-yl)cyclohexanecarboxylate (90.0 mg, 298 umol) in DMF (0.5 mL) was added. The reaction mixture was stirred at 25° C. for 0.5 hr. On completion, the mixture was quenched with ice water (5 mL) and filtered to give the title compound (105 mg, 73% yield) as a brown solid. LC-MS (ESI+) m/z 475.0 (M+H)⁺.

### Step 8—N-[6-ethyl-2-[4-(hydroxymethyl)cyclohexyl]indazol-5-yl]-6-(trifluoromethyl)pyridine-2-carboxamide

To a mixture of methyl 4-[6-ethyl-5-[[6-(trifluoromethyl)pyridine-2-carbonyl]amino]indazol-2-yl]cyclohexanecarboxylate (105.0 mg, 221.3 umol) in THF (5 mL) was added LiAlH₄ (16.8 mg, 442.5 umol) at 0° C. The mixture was then stirred at 25° C. for 1 hr. On completion, the reaction mixture was quenched with H₂O (1 mL) and 15% NaOH (6 mL) at 20° C. Then the mixture was dried over anhydrous sodium sulfate, filtered and concentrated under reduced pressure to give a residue to give the title compound (89.0 mg, 79% yield) as a brown solid. LC-MS (ESI+) m/z 447.0 (M+H)⁺.

### Step 9—N-[6-ethyl-2-(4-formylcyclohexyl)indazol-5-yl]-6-(trifluoromethyl)pyridine-2-carboxamide

To a solution of N-[6-ethyl-2-[4-(hydroxymethyl)cyclohexyl]indazol-5-yl]-6-(trifluoromethyl) pyridine-2-carboxamide (89.0 mg, 199.3 umol) in DCM (3 mL) was added DMP (126.8 mg, 299.0 umol) at 0° C. The mixture was then stirred at 25° C. for 2 hrs. On completion, the mixture was quenched with saturated solution of Na₂S₂O₃ (40 mL) and saturated solution of NaHCO₃ (30 mL), then extracted with DCM (40 mL×3). The combined organic phase was dried over anhydrous sodium sulfate, filtered and concentrated to give a residue. The crude product was triturated with PE (1 mL) at 25° C. for 10 mins to give the title compound (80.0 mg, 77% yield) as brown solid. ¹H NMR (400 MHz, DMSO-d₆) δ10.18 (s, 1H), 9.65 (s, 1H), 8.48-8.42 (m, 1H), 8.40 (d, J=7.6 Hz, 1H), 8.38 (s, 1H), 8.24 (s, 1H), 8.21 (d, J=7.2 Hz, 1H), 7.50 (s, 1H), 4.55-4.37 (m, 1H), 2.78 (q, J=7.6 Hz, 2H), 2.44-2.39 (m, 1H), 2.21 (d, J=12.4 Hz, 2H), 2.11 (d, J=11.6 Hz, 2H), 2.04-1.93 (m, 2H), 1.51-1.39 (m, 2H), 1.26 (t, J=7.6 Hz, 3H); LC-MS (ESI+) m/z 445.1 (M+H)⁺.

### N-[2-(4-formylcyclohexyl)-6-morpholino-indazol-5-yl]-6-(trifluoromethyl)pyridine-2-carboxamide (Intermediate CFM)

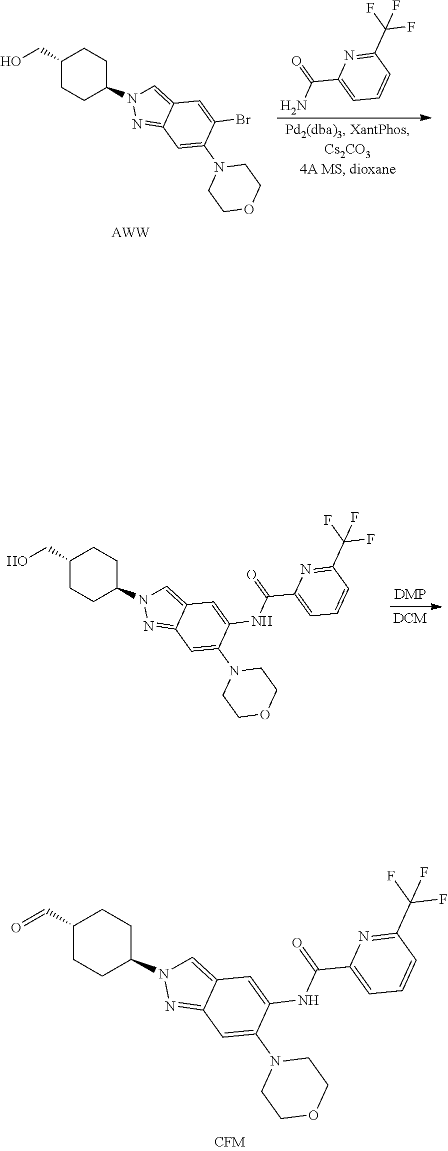 [SMILES: OC[C@H]1CC[C@H](n2cc3cc(Br)c(N4CCOCC4)cc3n2)CC1 ; NC(=O)c1cccc(C(F)(F)F)n1 ; "AWW" ; O=C(Nc1cc2cn([C@H]3CC[C@H](CO)CC3)nc2cc1N1CCOCC1)c1cccc(C(F)(F)F)n1 ; O=C[C@H]1CC[C@H](n2cc3cc(NC(=O)c4cccc(C(F)(F)F)n4)c(N4CCOCC4)cc3n2)CC1]

### Step 1—N-[2-[4-(hydroxymethyl)cyclohexyl]-6-morpholino-indazol-5-yl]-6-(trifluoromethyl)pyridine-2-carboxamide

To a mixture of [4-(5-bromo-6-morpholino-indazol-2-yl)cyclohexyl]methanol (1.20 g, 3.04 mmol, Intermediate AWW) and 6-(trifluoromethyl)pyridine-2-carboxamide (694 mg, 3.65 mmol, CAS #22245-84-7) in dioxane (20 mL) was added 4 Å molecular sieves (1.00 g, 3.04 mmol), Pd₂(dba)₃ (278 mg, 304 umol), Xantphos (352 mg, 608 umol) and Cs₂CO₃ (2.97 g, 9.13 mmol). The reaction mixture was then stirred at 100° C. for 12 hours. On completion, the reaction mixture was filtered and concentrated in vacuo. The residue was diluted with water (30 mL) and extracted with EA (2×30 mL). The combined organic layer was dried over Na2SO4, filtered and concentrated in vacuo to give a residue. The residue was purified by reverse phase (0.1% FA condition) to give the title compound (940 mg, 61% yield) as white solid. LC-MS (ESI⁺) m/z 504.3 (M+H).

### Step 2—N-[2-(4-formylcyclohexyl)-6-morpholino-indazol-5-yl]-6-(trifluoromethyl)pyridine-2-carboxamide

To a mixture of N-[2-[4-(hydroxymethyl)cyclohexyl]-6-morpholino-indazol-5-yl]-6-(trifluoromethyl) pyridine-2-carboxamide (100 mg, 198 umol) in DCM (3 mL) was added DMP (109 mg, 258 umol, 79.9 uL). The reaction mixture was then stirred at 25° C. for 1 hr. On completion, the reaction mixture was quenched with saturated Na₂S₂O₃ (3 mL) and saturated NaHCO₃ (3 mL) at 25° C., and then stirred for 30 minutes. The residue was diluted with water (10 mL) and extracted with DCM (2×20 mL). The combined organic layer was dried over Na₂SO₄, filtered and concentrated in vacuo to give the title compound (80.0 mg, 80% yield) as white solid. LC-MS (ESI⁺) m/z 502.2 (M+H)⁺.

### 1-(8-Bromoquinazolin-4-yl)-3-[(4-methoxyphenyl)methyl]hexahydropyrimidine-2,4-dione (Intermediate CFN)

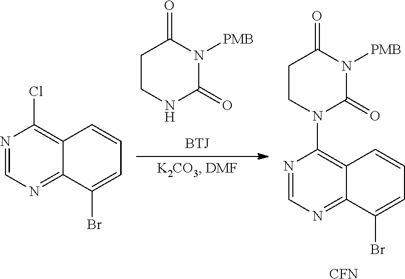

To a solution of 8-bromo-4-chloro-quinazoline (2.00 g, 8.21 mmol, CAS #125096-72-2) and 3-[(4-methoxyphenyl)methyl]hexahydropyrimidine-2,4-dione (2.89 g, 12.3 mmol, Intermediate BTJ) in DMF (25 mL) was added K₂CO₃ (3.41 g, 24.6 mmol), then the mixture was stirred at 60° C. for 10 hours. On completion, the mixture was filtered and the filtrate was extracted with water (200 mL) and EA (150 mL×3). The combined organic layers was dried over anhydrous Na₂SO₄, then concentrated in vacuo to give the residue. The residue was purified by reverse-phase (0.1% FA condition) to give the title compound (1.10 g, 30% yield) as a yellow solid. ¹H NMR (400 MHz, DMSO-d₆) δ 9.29 (s, 1H), 8.37-8.35 (m, 1H), 8.11-8.09 (m, 1H), 7.65-7.54 (m, 1H), 7.22 (d, J=8.8 Hz, 2H), 6.89-6.85 (m, 2H), 4.84 (s, 2H), 4.06 (s, 2H), 3.72 (s, 3H), 3.09-3.01 (m, 2H). LC-MS (ESI⁺) m/z 442.9 (M+H)⁺.

### 1-(8-Piperazin-1-ylquinazolin-4-yl)hexahydropyrimidine-2,4-dione (Intermediate CFO)

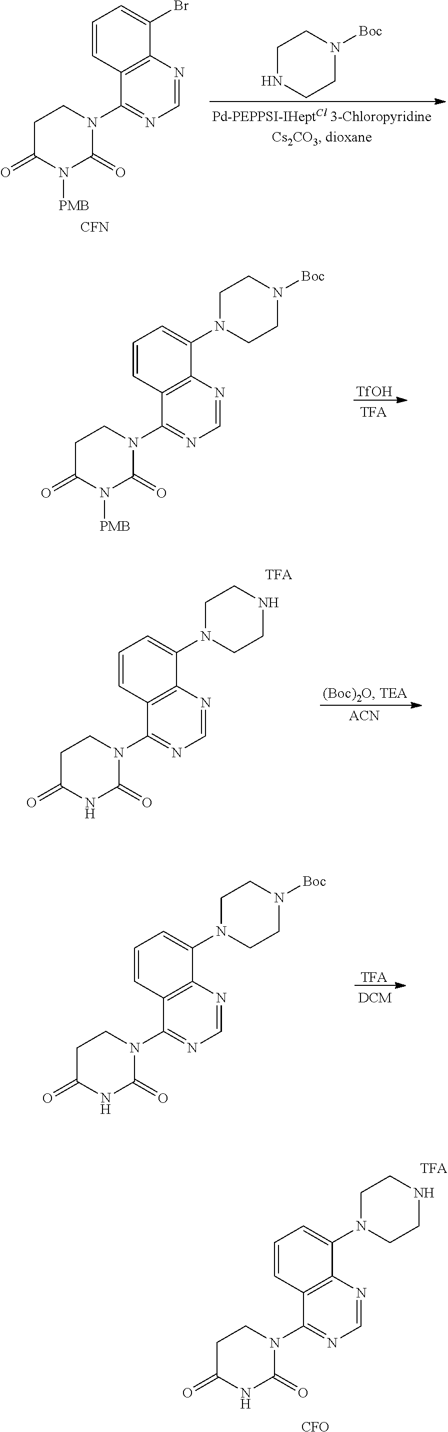

### Step 1—Tert-butyl 4-[4-[3-[(4-methoxyphenyl)methyl]-2,4-dioxo-hexahydropyrimidin-1-yl]quinazolin-8-yl]piperazine-1-carboxylate

To a solution of 1-(8-bromoquinazolin-4-yl)-3-[(4-methoxyphenyl)methyl]hexahydropyrimidine-2,4-dione (500 mg, 1.13 mmol, Intermediate CFN) and tert-butyl piperazine-1-carboxylate (316 mg, 1.70 mmol, CAS #143238-38-4) in dioxane (6 mL) was added Pd-PEPPSI-IHeptCl 3-Chloropyridine (110 mg, 113 umol) and Cs₂CO₃ (738 mg, 2.27 mmol). Then the mixture was stirred at 80° C. for 10 hours. On completion, the mixture was filtered and the filtrate was concentrated in vacuo to give the residue. The residue was purified by reverse-phase (0.1% FA condition) to give the title compound (230 mg, 37% yield) as a yellow solid. ¹H NMR (400 MHz, DMSO-d₆) δ 9.15 (s, 1H), 7.61-7.51 (m, 2H), 7.36-7.34 (m, 1H), 7.22 (d, J=8.4 Hz, 2H), 6.89-6.85 (m, 2H), 4.83 (s, 2H), 4.01 (s, 2H), 3.72 (s, 3H), 3.59 (s, 4H), 3.28 (s, 2H), 3.12-2.96 (m, 2H), 2.52-2.50 (m, 2H), 1.44 (s, 9H). LC-MS (ESI⁺) m/z 547.5 (M+H)⁺.

### Step 2—1-(8-Piperazin-1-ylquinazolin-4-yl)hexahydropyrimidine-2,4-dione

To a solution of tert-butyl 4-[4-[3-[(4-methoxyphenyl)methyl]-2,4-dioxo-hexahydropyrimidin-1-yl]quinazolin-8-yl]piperazine-1-carboxylate (200 mg, 365 umol) in TFA (5 mL) was added TfOH (2 mL, 22.6 mmol), then the mixture was stirred at 70° C. for 1 hour. On completion, the mixture was concentrated in vacuo to give the title compound (150 mg, 93% yield, TFA) as a brown solid.

### Step 3—Tert-butyl 4-[4-(2,4-dioxohexahydropyrimidin-1-yl)quinazolin-8-yl]piperazine-1-carboxylate

To a solution of 1-(8-piperazin-1-ylquinazolin-4-yl)hexahydropyrimidine-2,4-dione (150 mg, 340 umol, TFA) in DCM (4 mL) was added TEA (94.8 uL, 681 umol) and Boc₂O (117 uL, 510 umol), then the mixture was stirred at 25° C. for 10 hours. On completion, the mixture was concentrated in vacuo to give the residue. The residue was purified by prep-HPLC (column: Phenomenex luna C18 150*25 mm*10 um; mobile phase: [water(FA)-ACN]; B %: 26%-56%, 9 min) to give the title compound (90.0 mg, 62% yield) as a yellow solid. ¹H NMR (400 MHz, DMSO-d₆) δ 10.75 (s, 1H), 9.15 (s, 1H), 7.65-7.60 (m, 1H), 7.58-7.52 (m, 1H), 7.35 (d, J=6.8 Hz, 1H), 4.02 (s, 2H), 3.59 (s, 4H), 2.84 (s, 2H), 2.52-2.50 (m, 4H), 1.44 (s, 9H). LC-MS (ESI⁺) m/z 427.3 (M+H)⁺.

### Step 4—1-(8-Piperazin-1-ylquinazolin-4-yl)hexahydropyrimidine-2,4-dione

To a solution of tert-butyl 4-[4-(2,4-dioxohexahydropyrimidin-1-yl)quinazolin-8-yl]piperazine-1-carboxylate (90.0 mg, 211 umol) in DCM (2 mL) was added TFA (0.8 mL, 10.8 mmol), then the mixture was stirred at 25° C. for 1 hour. On completion, the mixture was concentrated in vacuo to give the title compound (90.0 mg, 96% yield, TFA) as a green solid. LC-MS (ESI⁺) m/z 327.1 (M+H)⁺.

### 1-(8-bromo-4-quinolyl)-3-[(4-methoxyphenyl)methyl]hexahydropyrimidine-2,4-dione (Intermediate CFP)

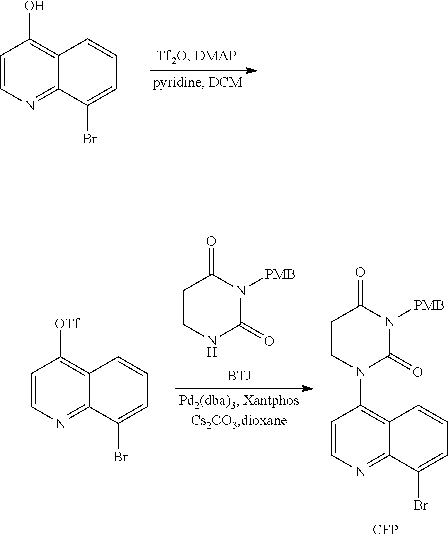

### Step 1—(8-Bromo-4-quinolyl) trifluoromethanesulfonate

To a mixture of 8-bromoquinolin-4-ol (0.500 g, 2.23 mmol, CAS #57798-00-2), pyridine (529 mg, 6.69 mmol) and DMAP (13.6 mg, 111 umol) in DCM (10 mL) was added Tf₂O (944 mg, 3.35 mmol) at 0° C. Then the reaction mixture was stirred at 25° C. for 2 hour. On completion, the reaction mixture was diluted with water (10 mL) and extracted with EA (2×20 mL). The combined organic layer was dried over Na₂SO₄, filtered and concentrated in vacuo to give the title compound (720 mg, 90% yield) as light yellow solid. ¹H NMR (400 MHz, DMSO-d₆) δ 9.19 (d, J=5.2 Hz, 1H), 8.60-8.55 (m, 1H), 8.38-8.32 (m, 1H), 8.06 (d, J=8.4 Hz, 1H), 7.91 (d, J=5.2 Hz, 1H).

### Step 2—1-(8-Bromo-4-quinolyl)-3-[(4-methoxyphenyl)methyl]hexahydropyrimidine-2,4-dione

To a mixture of (8-bromo-4-quinolyl) trifluoromethanesulfonate (500 mg, 1.40 mmol) and 3-[(4-methoxyphenyl)methyl]hexahydropyrimidine-2,4-dione (328 mg, 1.40 mmol, Intermediate BTJ) in dioxane (10 mL) was added Pd₂(dba)₃ (128 mg, 140 umol), Xantphos (162 mg, 280 umol) and Cs₂CO₃ (1.37 g, 4.21 mmol). The reaction mixture was then stirred at 80° C. for 4 hour. On completion, the residue was diluted with water (10 mL) and extracted with EA(2×10 mL). The combined organic layer was dried over Na₂SO₄, filtered and concentrated in vacuo to give a residue. The residue was purified by prep-HPLC (column: Waters xbridge 150*25 mm 10 um; mobile phase: [water (NH₄HCO₃)-ACN]; B %: 32%-62%, 8 min) to give the title compound (97.0 mg, 15% yield) as white solid. ¹H NMR (400 MHz, DMSO-d₆) δ 9.09 (d, J=4.4 Hz, 1H), 8.23-8.18 (m, 1H), 8.05-8.00 (m, 1H), 7.70 (d, J=4.4 Hz, 1H), 7.57-7.48 (m, 1H), 7.28-7.24 (m, 2H), 6.93-6.84 (m, 2H), 4.83 (s, 2H), 4.12-4.00 (m, 1H), 3.73 (s, 4H), 3.27-3.14 (m, 1H), 2.97-2.86 (m, 1H).

### 1-(8-piperazin-1-yl-4-quinolyl)hexahydropyrimidine-2,4-dione (Intermediate CFQ)

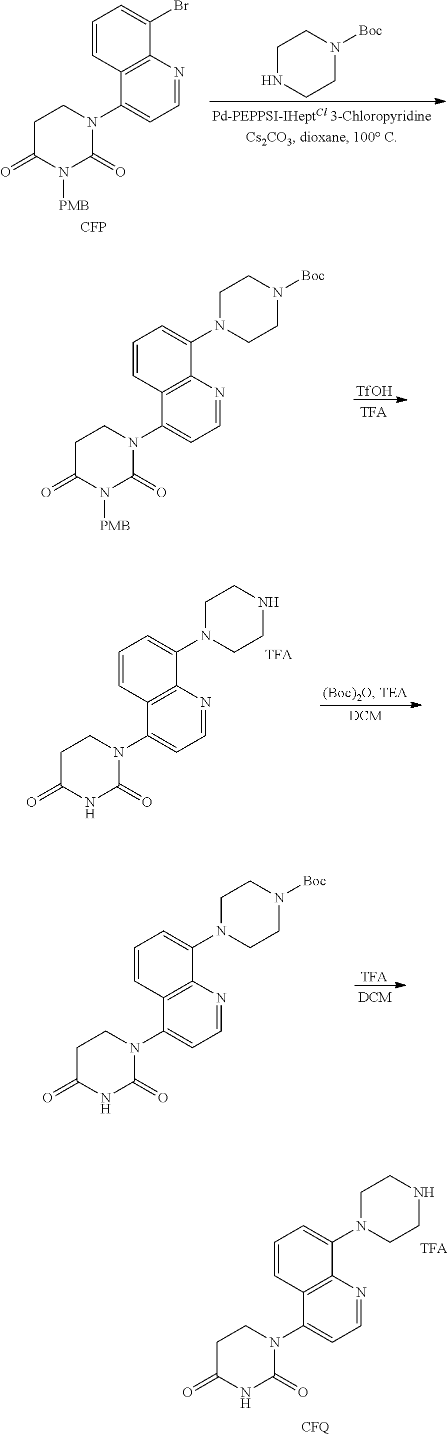

### Step 1—Tert-butyl 4-[4-[3-[(4-methoxyphenyl)methyl]-2,4-dioxo-hexahydropyrimidin-1-yl]-8-quinolyl]piperazine-1-carboxylate

A solution of 1-(8-bromo-4-quinolyl)-3-[(4-methoxyphenyl)methyl]hexahydropyrimidine-2,4-dione (468 mg, 1.06 mmol, Intermediate CFP), tert-butyl piperazine-1-carboxylate (237 mg, 1.28 mmol, CAS #57260-71-6), Cs₂CO₃ (1.04 g, 3.19 mmol) and Pd-PEPPSI-IHept^{Cl} 3-Chloropyridine (103 mg, 106 umol) in dioxane (7 mL) was stirred at 100° C. under N₂ for 2 hrs. On completion, the reaction mixture was diluted with EtOAc (80 mL), filtered and concentrated in vacuo. The residue was purified by column chromatography (SiO₂, PE:EA=3:1 to 2:3) to give the title compound (490 mg, 84% yield) as yellow solid. LC-MS (ESI⁺) m/z 546.3 (M+H)⁺.

### Step 2—1-(8-piperazin-1-yl-4-quinolyl)hexahydropyrimidine-2,4-dione

To a solution of tert-butyl 4-[4-[3-[(4-methoxyphenyl)methyl]-2,4-dioxo-hexahydropyrimidin-1-yl]-8-quinolyl]piperazine-1-carboxylate (470 mg, 861 umol) in TFA (3 mL) was added TfOH (1.02 g, 6.80 mmol). The reaction was stirred at 70° C. for 1 hr. On completion, the reaction mixture was concentrated in vacuo to give the title compound (378 mg, 99% yield, TFA) as brown oil. LC-MS (ESI⁺) m/z 326.2 (M+H)⁺.

### Step 3—Tert-butyl 4-[4-(2,4-dioxohexahydropyrimidin-1-yl)-8-quinolyl]piperazine-1-carboxylate

To a solution of 1-(8-piperazin-1-yl-4-quinolyl)hexahydropyrimidine-2,4-dione (378 mg, 860 umol, TFA) and TEA (174 mg, 1.72 mmol) in DCM (4 mL) was added Boc₂O (281 mg, 1.29 mmol) at 25° C. The reaction was then stirred at 25° C. for 1 hr. On completion, the reaction was diluted with DCM (100 mL) washed with water (50 mL×3). The organic layer was dried over Na₂SO₄, filtered and concentrated in vacuo. The residue was triturated with solvent (EA:PE=1:10, 20 mL) at 25° C. for 20 mins to give the title compound (312 mg, 85% yield) as brown solid. ¹H NMR (400 MHz, DMSO-d₆) δ 10.57 (s, 1H), 8.92 (d, J=4.4 Hz, 1H), 7.64-7.46 (m, 3H), 7.19 (d, J=7.2 Hz, 1H), 4.02-3.93 (m, 1H), 3.69-3.63 (m, 1H), 3.59 (s, 4H), 3.47-3.33 (m, 2H), 3.29-3.20 (m, 2H), 3.05-2.94 (m, 1H), 2.74-2.66 (m, 1H), 1.47-1.42 (m, 9H).

### Step 4—1-(8-piperazin-1-yl-4-quinolyl)hexahydropyrimidine-2,4-dione

To a solution of tert-butyl 4-[4-(2,4-dioxohexahydropyrimidin-1-yl)-8-quinolyl]piperazine-1-carboxylate (50 mg, 117 umol) in DCM (1 mL) was added TFA (308 mg, 2.70 mmol) at 25° C. The reaction was then stirred at 25° C. for 1 hr. On completion, the reaction mixture was concentrated in vacuo to give the title compound (51 mg, 98% yield, TFA) as brown oil. LC-MS (ESI⁺) m/z 326.2 (M+H)⁺.

### 3-(3-Methyl-2-oxo-4-((1s, 3s)-3-(piperidin-4-yloxy)cyclobutyl)-2,3-dihydro-1H-benzo[d]imidazol-1-yl)piperidine-2,6-dione (Intermediate CFR)

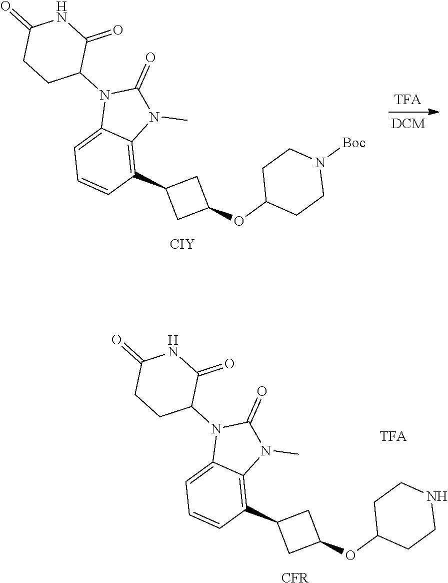

A mixture of tert-butyl 4-[3-[1-(2,6-dioxo-3-piperidyl)-3-methyl-2-oxo-benzimidazol-4-yl]cyclobutoxy]piperidine-1-carboxylate (25.0 mg, 48.7 umol, Intermediate CIY) and TFA (513 mg, 4.50 mmol) in DCM (1 mL) was stirred at 25° C. for 1 hour. On completion, the reaction was concentrated in vacuo to give the title compound (25.0 mg, 97% yield) as yellow oil. LC-MS (ESI⁺) m/z 413.3 (M+H)⁺.

### Tert-butyl 4-((1r, 3r)-3-(1-(2,6-dioxopiperidin-3-yl)-3-methyl-2-oxo-2,3-dihydro-1H-benzo [d]imidazol-4-yl)cyclobutoxy)piperidine-1-carboxylate (Intermediate CFS)

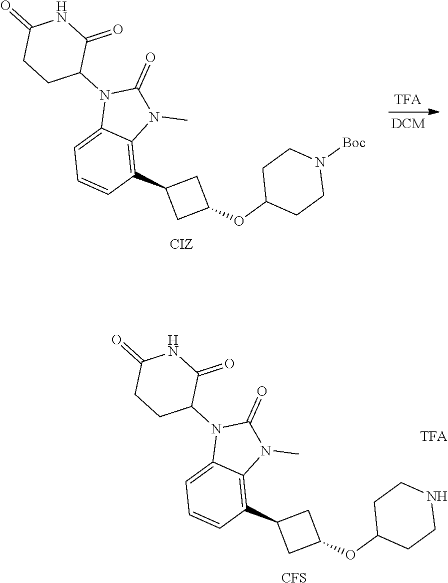

To a solution of tert-butyl 4-[3-[1-(2,6-dioxo-3-piperidyl)-3-methyl-2-oxo-benzimidazol-4-yl]cyclobutoxy]piperidine-1-carboxylate (15.0 mg, 29.2 umol, Intermediate CIZ) in DCM (1 mL) was added TFA (0.3 mL), then the reaction mixture was stirred at 25° C. for 2 hours. On completion, the reaction was concentrated in vacuo to give the title compound (15.0 mg, 97.3% yield) as yellow oil. LC-MS (ESI⁺) m/z 413.2 (M+H)⁺.

### N-[6-(azetidin-1-yl)-2-(4-formylcyclohexyl)indazol-5-yl]-6-(trifluoromethyl)pyridine-2-carboxamide (Intermediate CFT)

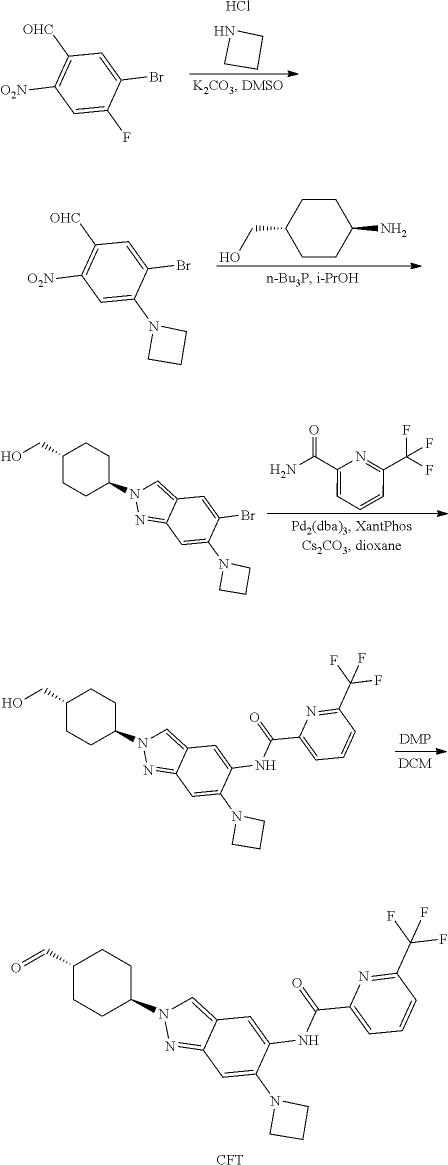

### Step 1—4-(Azetidin-1-yl)-5-bromo-2-nitro-benzaldehyde

To a solution of azetidine (2.30 g, 40.3 mmol, CAS #503-29-7) and 5-bromo-4-fluoro-2-nitro-benzaldehyde (2 g, 8.06 mmol, CAS #213382-45-7) in DMSO (10 mL) was added TEA (4.08 g, 40.3 mmol). The mixture was stirred at 80° C. for 1 hr. On completion, the residue was diluted with water (30 mL) and extracted with EA (30 mL×2). The combined organic layer was dried over Na₂SO₄, filtered and concentrated in vacuo to give the title compound (2.0 g, 78% yield) as a brown oil. ¹H NMR (400 MHz, CDCl₃) δ 8.50 (s, 1H), 8.10 (s, 1H), 3.65-3.58 (m, 2H), 2.45 (t, J=7.3 Hz, 2H), 1.70 (q, J=7.2 Hz, 2H).

### Step 2—[4-[6-(Azetidin-1-yl)-5-bromo-indazol-2-yl]cyclohexyl]methanol

To a solution of 4-(azetidin-1-yl)-5-bromo-2-nitro-benzaldehyde (1.8 g, 6.31 mmol) and (4-aminocyclohexyl)methanol (815 mg, 6.31 mmol) in i-PrOH (20 mL), then the reaction mixture was stirred at 80° C. for 2 hrs under N₂. Then the mixture was cooled to 25° C. and tributylphosphane (3.19 g, 15.7 mmol) was added. Then the mixture was stirred at 80° C. for 2 hrs under N₂. On completion, the residue was purified by column chromatography (SiO₂, PE:EA=6:1 to PE:EA=2:1, PE:EA=2:1, P1:Rf=0.3) to give the title compound (850 mg, 36% yield) as yellow solid. ¹H NMR (400 MHz, DMSO-d₆) δ 8.17 (s, 1H), 7.83 (s, 1H), 6.67 (s, 1H), 4.48 (t, J=5.2 Hz, 1H), 4.38-4.25 (m, 1H), 3.92 (t, J=7.2 Hz, 4H), 3.27 (t, J=5.6 Hz, 2H), 2.17 (quin, J=7.2 Hz, 2H), 2.08 (d, J=10.4 Hz, 2H), 1.92-1.76 (m, 4H), 1.52-1.38 (m, 1H), 1.19-1.03 (m, 2H); LC-MS (ESI⁺) m/z 364.0 (M+H)⁺.

### Step 3—N-[6-(azetidin-1-yl)-2-[4-(hydroxymethyl)cyclohexyl]indazol-5-yl]-6-(trifluoromethyl)pyridine-2-carboxamide

A mixture of [4-[6-(azetidin-1-yl)-5-bromo-indazol-2-yl]cyclohexyl]methanol (200 mg, 549 umol), 6-(trifluoromethyl)pyridine-2-carboxamide (135 mg, 713 umol, CAS #22245-84-7), Pd₂(dba)₃ (50.2 mg, 54.9 umol), Xantphos (63.5 mg, 109 umol) and Cs₂CO₃ (357 mg, 1.10 mmol) in dioxane (4 mL) was degassed and purged with N₂ three times. Then the mixture was stirred at 100° C. for 8 hrs under N₂ atmosphere. On completion, the reaction mixture was filtered and concentrated in vacuo. The residue was diluted with DCM (15 mL) and extracted with DCM (15 mL×2). The combined organic layers were washed with H₂O (10 mL×3), filtered and concentrated in vacuo. The residue was purified by column chromatography (SiO₂, PE:EA=5:1 to PE:EA=0:1, PE:EA=1:1, P1:Rf=0.2). Then, the residue was purified by prep-HPLC (column: Phenomenex luna C18 150*25 mm*10 um; mobile phase: [water (FA)-ACN]; B %: 37%-67%, 10.5 min) to give the title compound (60 mg, 23% yield) as a yellow solid. ¹H NMR (400 MHz, DMSO-d₆) δ 10.18 (s, 1H), 8.48-8.37 (m, 3H), 8.28 (s, 1H), 8.22 (dd, J=0.8, 7.6 Hz, 1H), 6.92 (s, 1H), 4.48 (t, J=5.2 Hz, 1H), 4.40-4.29 (m, 1H), 3.87 (t, J=7.2 Hz, 4H), 3.30-3.25 (m, 2H), 2.25 (quin, J=7.2 Hz, 2H), 2.12 (dd, J=3.6, 9.2 Hz, 2H), 1.94-1.82 (m, 4H), 1.54-1.42 (m, 1H), 1.21-1.08 (m, 2H).

### Step 4—N-[6-(azetidin-1-yl)-2-(4-formylcyclohexyl)indazol-5-yl]-6-(trifluoromethyl)pyridine-2-carboxamide

To a solution of N-[6-(azetidin-1-yl)-2-[4-(hydroxymethyl)cyclohexyl]indazol-5-yl]-6-(trifluoromethyl) pyridine-2-carboxamide (45.0 mg, 95.0 umol) in DMSO (1 mL) was added IBX (79.8 mg, 285 umol). The mixture was then stirred at 50° C. for 3 hrs. On completion, the reaction mixture was quenched with saturated Na₂S₂O₃ (5 mL) and saturated NaHCO₃ (5 mL) at 25° C. and stirred for 5 minutes. The mixture was then extracted with DCM (20 mL×2) and washed with H₂O (10 mL×2). The reaction mixture was concentrated under reduced pressure to give the title compound (40 mg, 80% yield) as a yellow solid. LC-MS (ESI⁺) m/z 472.0 (M+H)⁺.

### N-[2-(4-formylcyclohexyl)-6-pyrrolidin-1-yl-indazol-5-yl]-6-(trifluoromethyl)pyridine-2-carboxamide (Intermediate CFU)

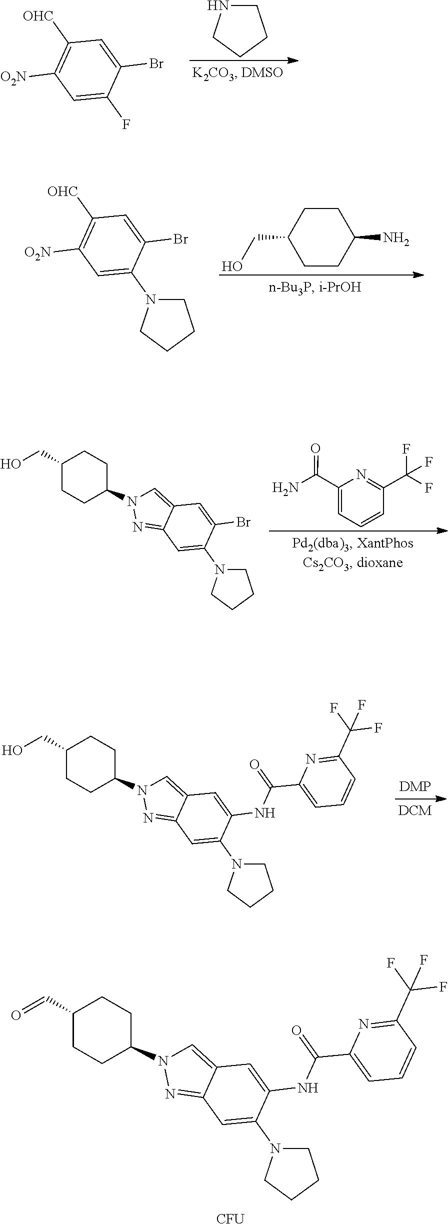

### Step 1—5-Bromo-2-nitro-4-pyrrolidin-1-yl-benzaldehyde

To a solution of 5-bromo-4-fluoro-2-nitro-benzaldehyde (5.00 g, 20.1 mmol, CAS #213382-45-7) and pyrrolidine (2.87 g, 40.3 mmol, CAS #123-75-1) in DMF (80 mL) was added K₂CO₃ (5.57 g, 40.3 mmol), then the mixture was stirred at 50° C. for 1 hour. On completion, the mixture was filtered and washed with water (150 mL) and extracted with EA (200 mL×3). The combined organic layer was dried over anhydrous Na₂SO₄, then concentrated in vacuo to give the residue. The residue was purified by column chromatography (SiO₂, PE:EA=20:1 to 12:1) to give the title compound (5.90 g, 97% yield) as a red solid. ¹H NMR (400 MHz, DMSO-d₆) δ 8.91 (d, J=2.8 Hz, 2H), 8.51 (d, J=2.8 Hz, 1H), 8.49 (d, J=2.8 Hz, 1H), 8.08 (d, J=8.8 Hz, 2H). LC-MS (ESI⁺) m/z 299.0 (M+H)⁺.

### Step 2—[4-(5-Bromo-6-pyrrolidin-1-yl-indazol-2-yl)cyclohexyl]methanol

To a solution of 5-bromo-2-nitro-4-pyrrolidin-1-yl-benzaldehyde (1.50 g, 5.01 mmol) in IPA (15 mL) was added (4-aminocyclohexyl)methanol (1.94 g, 15.0 mmol, CAS #1467-84-1), and the mixture stirred at 80° C. for 4 hours. Next, tributylphosphane (1.52 g, 7.52 mmol) was added at 25° C. and the mixture was stirred at 80° C. for 5 hours. On completion, the mixture was concentrated in vacuo to give the residue. The residue was purified by reverse-phase (0.1% FA condition) to give the title compound (2.20 g, 38% yield) as a white solid. ¹H NMR (400 MHz, DMSO-d₆) δ 8.23 (s, 1H), 7.90-7.88 (m, 1H), 7.13 (s, 1H), 4.48-4.46 (m, 1H), 4.38-4.31 (m, 1H), 3.29-3.26 (m, 2H), 3.19-3.16 (m, 4H), 2.11-2.08 (m, 2H), 1.91-1.82 (m, 7H), 1.48-1.43 (m, 2H), 1.20-1.05 (m, 2H) LC-MS (ESI⁺) m/z 380.0 (M+H)⁺.

### Step 3—N-[2-[4-(hydroxymethyl)cyclohexyl]-6-pyrrolidin-1-yl-indazol-5-yl]-6-(trifluoromethyl)-pyridine-2-carboxamide

To a solution of [4-(5-bromo-6-pyrrolidin-1-yl-indazol-2-yl)cyclohexyl]methanol (500 mg, 1.32 mmol) and 6-(trifluoromethyl)pyridine-2-carboxamide (376 mg, 1.98 mmol, CAS #22245-84-7) in dioxane (6 mL) was added Pd₂(dba)₃ (121 mg, 132 umol), Cs₂CO₃ (861 mg, 2.64 mmol) and Xantphos (76.4 mg, 132 umol), then the mixture was stirred at 110° C. for 16 hours. On completion, the mixture was filtered and extracted with EA (10 mL×3), then the combined organic phase was dried over anhydrous Na₂SO₄ and concentrated in vacuo to give the residue. The residue was purified by reverse phase (0.1% FA condition) to give the title compound (560 mg, 86% yield) as a yellow solid. ¹H NMR (400 MHz, DMSO-d₆) δ 10.89 (s, 1H), 8.70 (s, 1H), 8.50-8.45 (m, 1H), 8.43-8.37 (m, 1H), 8.34 (s, 1H), 8.21 (d, J=7.6 Hz, 1H), 7.43 (s, 1H), 4.50-4.48 (m, 1H), 4.40-4.34 (m, 1H), 3.30-3.27 (m, 2H), 3.07 (s, 4H), 2.14-2.10 (m, 2H), 1.98-1.97 (m, 4H), 1.89-1.87 (m, 4H), 1.49-1.44 (m, 1H), 1.19-1.12 (m, 2H). LC-MS (ESI⁺) m/z 488.2 (M+H)⁺.

### Step 4—N-[2-(4-formylcyclohexyl)-6-pyrrolidin-1-yl-indazol-5-yl]-6-(trifluoromethyl)pyridine-2-carboxamide

To a solution of N-[2-[4-(hydroxymethyl)cyclohexyl]-6-pyrrolidin-1-yl-indazol-5-yl]-6-(trifluoro-methyl)pyridine-2-carboxamide (60.0 mg, 123 umol) in DCM (2 mL) was added DMP (78.3 mg, 184 umol) at 0° C., then the mixture was stirred at 25° C. for 1 hour. On completion, the mixture was quenched with sat. Na₂S₂O₃ (3 mL) and sat. NaHCO₃ (3 mL), then the mixture was extracted with DCM (10 mL×3). The combined organic layer was dried over anhydrous Na₂SO₄, filtered and concentrated in vacuo to give the title compound (40.0 mg, 66% yield) as a black oil. LC-MS (ESI⁺) m/z 486.1 (M+H)⁺.

### 3-[4-(3,8-diazabicyclo[3.2.1]octan-8-yl)-3-methyl-2-oxo-benzimidazol-1-yl] piperidine-2,6-dione (Intermediate CET)

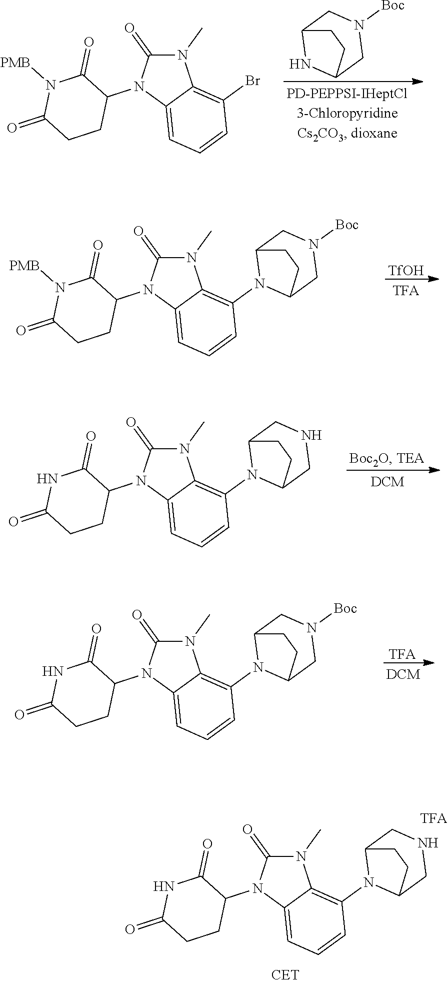

Step 1—Tert-butyl 8-(1-(1-(4-methoxybenzyl)-2,6-dioxopiperidin-3-yl)-3-methyl-2-oxo-2,3-dihydro-1H-benzo[d]imidazol-4-yl)-3,8-diazabicyclo[3.2.1]octane-3-carboxylate 4 To a solution of 3-(4-bromo-3-methyl-2-oxo-benzimidazol-1-yl)-1-[(4-methoxyphenyl)methyl]piperidine-2,6-dione (500 mg, 1.09 mmol, synthesized via Steps 1-4 of Intermediate HP), tert-butyl 3,8-diazabicyclo[3.2.1]octane-3-carboxylate (301 mg, 1.42 mmol), PD-PEPPSIIHeptCl 3-Chloropyridine (105 mg, 109 umol) and Cs₂CO₃ (710.92 mg, 2.18 mmol) in dioxane (3 mL) was de-gassed and then heated to 100° C. for 16 hours under N₂. On completion, the reaction mixture was diluted with EA (60 mL), and extracted with H₂O (10 mL×3). The organic layer was separated, dried over Na2S04, filtered and concentrated in vacuo. The residue was purified by column chromatography (SiO₂, Petroleum ether/Ethyl acetate=2/1 to 1/1) to give the title compound (270 mg, 41% yield) as yellow solid. LC-MS (ESI⁺) m/z 590.2 (M+H)⁺.

### Step 2—3-(4-(3,8-Diazabicyclo[3.2.1]octan-8-yl)-3-methyl-2-oxo-2,3-dihydro-1H-benzo[d]imidazol-1-yl)piperidine-2,6-dione

To a solution of tert-butyl 8-[1-[1-[(4-methoxyphenyl)methyl]-2,6-dioxo-3-piperidyl]-3-methyl-2-oxo-benzimidazol-4-yl]-3,8-diazabicyclo[3.2.1]octane-3-carboxylate (70 mg, 118 umol) in TFA (0.6 mL) was added TfOH (0.2 mL) 25° C. Then the reaction was stirred at 70° C. for 1 h. On completion, the reaction mixture was concentrated in vacuo to give the title compound (57 mg, 99% yield) as brown oil. LC-MS (ESI⁺) m/z 370.2 (M+H)⁺.

### Step 3—Tert-butyl 8-(1-(2,6-dioxopiperidin-3-yl)-3-methyl-2-oxo-2,3-dihydro-1H-benzo[d]imidazol-4-yl)-3,8-diazabicyclo[3.2.1]octane-3-carboxylate

To a solution of 3-[4-(3,8-diazabicyclo[3.2.1]octan-8-yl)-3-methyl-2-oxo-benzimidazol-1-yl]piperidine-2,6-dione (57 mg, 117 umol) in DCM (2 mL) was added TEA (0.3 mL) until the pH=8. Then, Boc2O (30.88 mg, 141 umol) was added into above mixture at 0° C. The reaction was then stirred at 25° C. for 1 h. On completion, the reaction mixture was diluted with DCM (30 mL), and extracted with water (20 mL×2). The organic layer was separated, dried over Na₂SO₄ and concentrated in vacuo. The residue was purified by reverse phase (FA) to give the title compound (50.0 mg, 90% yield) as yellow solid. LC-MS (ESI⁺) m/z 470.2 (M+H)⁺.

### Step 4—3-(4-(3,8-Diazabicyclo[3.2.1]octan-8-yl)-3-methyl-2-oxo-2,3-dihydro-1H-benzo[d]imidazol-1-yl)piperidine-2,6-dione

To a solution of tert-butyl 8-[1-(2,6-dioxo-3-piperidyl)-3-methyl-2-oxo-benzimidazol-4-yl]-3,8-diazabicyclo[3.2.1]octane-3-carboxylate (50 mg, 106 umol) in DCM (2 mL) was added TFA (12.14 mg, 106.49 umol) at 25° C. The reaction was then stirred at 25° C. for 1 h. On completion, the reaction mixture was concentrated in vacuo to give the title compound (50 mg, TFA salt, 95% yield) as yellow solid. LC-MS (ESI⁺) m/z 370.2 (M+H)⁺.

### N-[2-(4-formylcyclohexyl)-6-isopropyl-indazol-5-yl]-6-(trifluoromethyl)pyridine-2-carboxamide (Intermediate CFV)

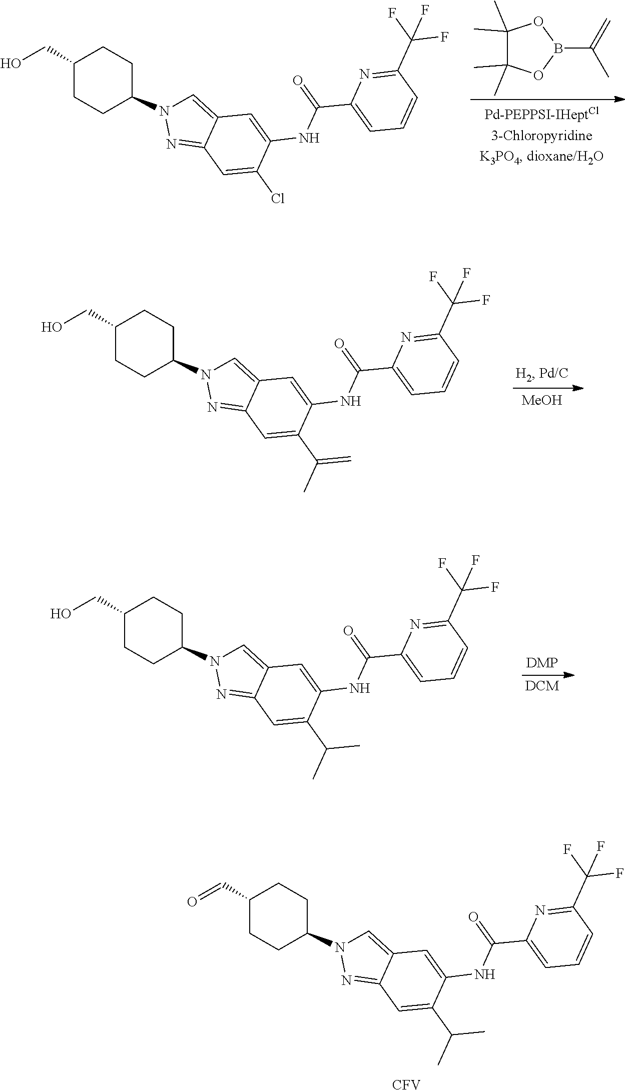

### Step 1—N-[2-[4-(hydroxymethyl)cyclohexyl]-6-isopropenyl-indazol-5-yl]-6-(trifluoromethyl)pyridine-2-carboxamide

To a solution of N-[6-chloro-2-[4-(hydroxymethyl)cyclohexyl]indazol-5-yl]-6-(trifluoromethyl) pyridine-2-carboxamide (1.00 g, 2.21 mmol, via Steps 1-3 of Intermediate BPQ) and 2-isopropenyl-4,4,5,5-tetramethyl-1,3,2-dioxaborolane (742 mg, 4.42 mmol, CAS #126726-62-3) in dioxane (15 mL) and H₂O (1.5 mL) was added K₃PO₄ (1.41 g, 6.62 mmol) and [2-(2-aminophenyl)phenyl]-chloro-palladium; dicyclohexyl-[3-(2,4,6-triisopropylphenyl)phenyl]phosphane (173 mg, 220 umol). Then the mixture was purged with N₂ for three times and stirred at 90° C. for 12 hours. On completion, the mixture was concentrated in vacuo to give the residue. The residue was purified by prep-HPLC (column: Phenomenex Luna C18 200*40 mm*10 um; mobile phase: [water(FA)-ACN]; B %: 50%-80%, 10 min) to give the title compound (440 mg, 43% yield) as a brown solid. ¹H NMR (400 MHz, DMSO-d₆) δ 10.41 (s, 1H), 8.70 (s, 1H), 8.46 (d, 2H), 8.40 (t, 1H), 8.20 (d, J=7.6 Hz, 1H), 7.51 (s, 1H), 5.51 (s, 1H), 5.14 (s, 1H), 4.52-4.48 (m, 1H), 4.43-4.40 (m, 1H), 3.30-3.27 (m, 2H), 2.52-2.48 (m, 3H), 2.15 (s, 2H), 1.96-1.85 (m, 4H), 1.54-1.43 (m, 1H), 1.22-1.10 (m, 2H). LC-MS (ESI⁺) m/z 459.1 (M+H)⁺.

### Step 2—N-[2-[4-(hydroxymethyl)cyclohexyl]-6-isopropyl-indazol-5-yl]-6-(trifluoromethyl)pyridine-2-carboxamide

To a solution of N-[2-[4-(hydroxymethyl)cyclohexyl]-6-isopropenyl-indazol-5-yl]-6-(trifluoromethyl) pyridine-2-carboxamide (100 mg, 218 umol) in MeOH (3 mL) was added Pd/C (30 mg, 10 wt %), then purged with H₂ (30.0 mg, 14.9 mmol) for three times, then the mixture was stirred at 25° C. for 2 hr under H₂ (15 psi). On completion, the mixture was filtered and concentrated in vacuo to give the title compound (90 mg, 89.61% yield) as a yellow solid. ¹H NMR (400 MHz, DMSO-d₆) δ 10.22 (s, 1H), 8.45-8.35 (m, 3H), 8.21-8.19 (m, 1H), 8.13 (s, 1H), 7.54 (s, 1H), 4.49 (t, J=5.2 Hz, 1H), 4.46-4.37 (m, 1H), 3.30-3.26 (m, 2H), 2.14-2.12 (m, 2H), 1.97-1.84 (m, 4H), 1.55-1.43 (m, 1H), 1.29 (d, J=6.8 Hz, 6H), 1.24-1.10 (m, 3H). LC-MS (ESI⁺) m/z 461.1 (M+H)⁺.

### Step 3—N-[2-(4-formylcyclohexyl)-6-isopropyl-indazol-5-yl]-6-(trifluoromethyl)pyridine-2-carboxamide

To a solution of N-[2-[4-(hydroxymethyl)cyclohexyl]-6-isopropyl-indazol-5-yl]-6-(trifluoromethyl) pyridine-2-carboxamide (50.0 mg, 108 umol) in DCM (2 mL) was added DMP (69.0 mg, 162 umol) at 0° C., then the mixture was stirred at 25° C. for 1 hour. On completion, the mixture was diluted with DCM (2 mL), then quenched with sat.Na₂S₂O₃ (2 mL) and sat.NaHCO₃ (2 mL), and the mixture was stirred at 25° C. for 10 minutes. Then the mixture was extracted with DCM (2 mL×3). Then the combined organic layers were dried over anhydrous Na₂SO₄, filtered and concentrated in vacuo to give the title compound (49.0 mg, 98% yield) as a yellow solid. LC-MS (ESI⁺) m/z 459.1 (M+H)⁺.

### N-[2-(4-formylcyclohexyl)-6-methyl-indazol-5-yl]-6-(trifluoromethyl)pyrazine-2-carboxamide (Intermediate CFW)

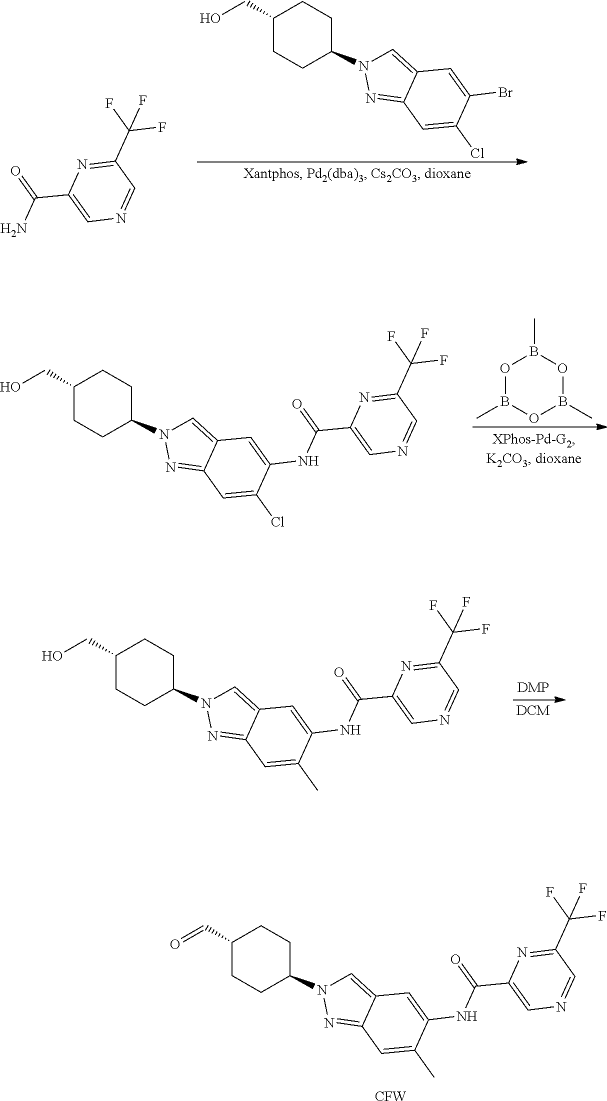

### Step 1—N-[6-chloro-2-[4-(hydroxymethyl)cyclohexyl]indazol-5-yl]-6-(trifluoromethyl)pyrazine-2-carboxamide

To a solution of [4-(5-bromo-6-chloro-indazol-2-yl)cyclohexyl]methanol (500 mg, 1.45 mmol, synthesized via Steps 1-2 of Intermediate BPQ) and 6-(trifluoromethyl)pyrazine-2-carboxamide (417 mg, 2.18 mmol, synthesized via Steps 1-2 of Intermediate CEO) in dioxane (5 mL) was added Pd₂(dba)₃ (133 mg, 145 umol), Xantphos (84.1 mg, 145 umol) and Cs₂CO₃ (948 mg, 2.91 mmol), then the mixture was stirred at 100° C. for 8 hours. On completion, the mixture was filtered and extracted with H₂O (10 mL) and EA (10 mL×3), dried over anhydrous Na₂SO₄, then concentrated in vacuo to give the residue. The residue was purified by column chromatography (SiO₂, PE:EA=15:1 to 1:5) give the title compound (413 mg, 62% yield) as a yellow solid. 1H NMR (400 MHz, DMSO-d₆) δ 10.41 (s, 1H), 9.61 (s, 1H), 9.51 (s, 1H), 8.54 (s, 1H), 8.43 (s, 1H), 7.92 (s, 1H), 4.51-4.42 (m, 2H), 3.30-3.27 (m, 2H), 2.17-2.14 (m, 2H), 1.95-1.85 (m, 4H), 1.55-1.43 (m, 1H), 1.23-1.12 (m, 2H). LC-MS (ESI⁺) m/z 454.1 (M+H)⁺.

### Step 2—N-[2-[4-(hydroxymethyl)cyclohexyl]-6-methyl-indazol-5-yl]-6-(trifluoromethyl)pyrazine-2-carboxamide

To a solution of 2,4,6-trimethyl-1,3,5,2,4,6-trioxatriborinane (110 mg, 881 umol) and N-[6-chloro-2-[4-(hydroxymethyl)cyclohexyl]indazol-5-yl]-6-(trifluoromethyl)pyrazine-2-carboxamide (200 mg, 440 umol) in dioxane (2 mL) was added K₂CO₃ (182 mg, 1.32 mmol) and XPhos-Pd-G₂ (34.6 mg, 44.0 umol), then the mixture was stirred at 90° C. for 8 hours under N₂ atmosphere. On completion, the mixture was filtered, then washed with H₂O (3 mL) and extracted with EA (5 mL×3). The combined organic layer was dried over anhydrous Na₂SO₄, and concentrated in vacuo to give the residue. The residue was purified by column chromatography (SiO₂, PE:EA=10:1 to 1:5) to give the title compound (180 mg, 94% yield) as a yellow solid. 1H NMR (400 MHz, DMSO-d₆) δ 10.19 (s, 1H), 9.58 (s, 1H), 9.48 (s, 1H), 8.37 (s, 1H), 7.96 (s, 1H), 7.51 (s, 1H), 4.50-4.47 (m, 1H), 4.40-4.38 (m, 1H), 3.30-3.27 (m, 2H), 2.36 (s, 3H), 2.16-2.13 (m, 2H), 1.91-1.88 (m, 4H), 1.51-1.46 (m, 1H), 1.20-1.19 (m, 1H), 1.15-1.13 (m, 1H). LC-MS (ESI⁺) m/z 434.1 (M+H)⁺.

### Step 3—N-[2-(4-formylcyclohexyl)-6-methyl-indazol-5-yl]-6-(trifluoromethyl)pyrazine-2-carboxamide

To a solution of N-[2-[4-(hydroxymethyl)cyclohexyl]-6-methyl-indazol-5-yl]-6-(trifluoromethyl) pyrazine-2-carboxamide (70.0 mg, 161 umol) in DCM (2 mL) was added DMP (102 mg, 242 umol,) at 0° C., then the mixture was stirred at 25° C. for 1 hour. On completion, the mixture was quenched with sat. Na₂S₂O₃ (1 mL) and sat. NaHCO₃ (1 mL), then the mixture was stirred at 25° C. for 0.5 hour. On completion, the mixture was extracted with DCM (2 mL×3), dried over anhydrous Na₂SO₄, then concentrated in vacuo to give the title compound (60.0 mg, 86% yield) as a yellow solid. LC-MS (ESI⁺) m/z 432.1 (M+H)⁺.

### 1-Methyl-3-(3-methyl-2-oxo-4-piperazin-1-yl-benzimidazol-1-yl)piperidine-2,6-dione (Intermediate CFX)

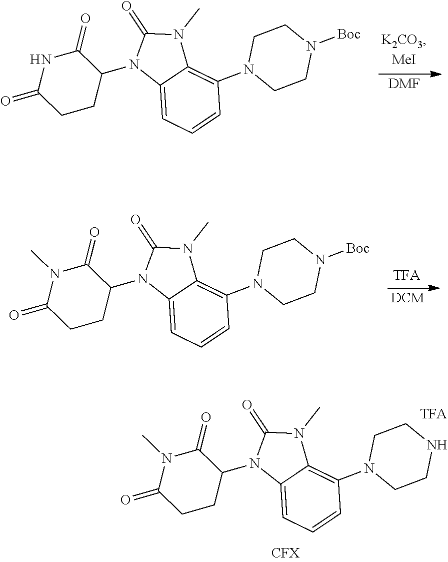

### Step 1—Tert-butyl 4-[3-methyl-1-(1-methyl-2,6-dioxo-3-piperidyl)-2-oxo-benzimidazol-4-yl]piperazine-1-carboxylate

To a solution of tert-butyl 4-[1-(2,6-dioxo-3-piperidyl)-3-methyl-2-oxo-benzimidazol-4-yl]piperazine-1-carboxylate (200 mg, 450 umol, synthesized via Steps 1-6 of Intermediate BAI) in DMF (4 mL) was added K₂CO₃ (124 mg, 901 umol) and CH₃I (96.0 mg, 676 umol), then the mixture was stirred at 60° C. for 4 hours. On completion, the mixture was diluted with sat. NH₄CL (50 mL) and extracted with EA (25 mL×3). Then the combined organic layers were dried over anhydrous Na₂SO₄, filtered and concentrated in vacuo to give the title compound (160 mg, 77% yield) as a brown solid. ¹H NMR (400 MHz, DMSO-d₆) δ 7.00-6.88 (m, 3H), 5.42 (d, J=12.8 Hz, 1H), 3.63 (s, 3H), 3.12-3.01 (m, 6H), 2.98-2.94 (m, 2H), 2.81-2.74 (m, 2H), 2.72-2.65 (m, 2H), 2.60-2.52 (m, 2H), 2.05-1.96 (m, 1H), 1.43 (s, 9H). LC-MS (ESI⁺) m/z 458.1 (M+H)⁺.

### Step 2—1-Methyl-3-(3-methyl-2-oxo-4-piperazin-1-yl-benzimidazol-1-yl)piperidine-2,6-dione

To a solution of tert-butyl 4-[3-methyl-1-(1-methyl-2,6-dioxo-3-piperidyl)-2-oxo-benzimidazol-4-yl]piperazine-1-carboxylate (80.0 mg, 174 umol) in DCM (2 mL) was added TFA (0.5 mL, 6.75 mmol), then the mixture was stirred at 25° C. for 1 hour. On completion, the mixture was concentrated in vacuo to give the title compound (80.0 mg, 97% yield, TFA) as a black brown oil. LC-MS (ESI⁺) m/z 358.1 (M+H)⁺.

### N-[6-acetyl-2-(4-formylcyclohexyl)indazol-5-yl]-6-(trifluoromethyl)pyridine-2-carboxamide (Intermediate CFY)

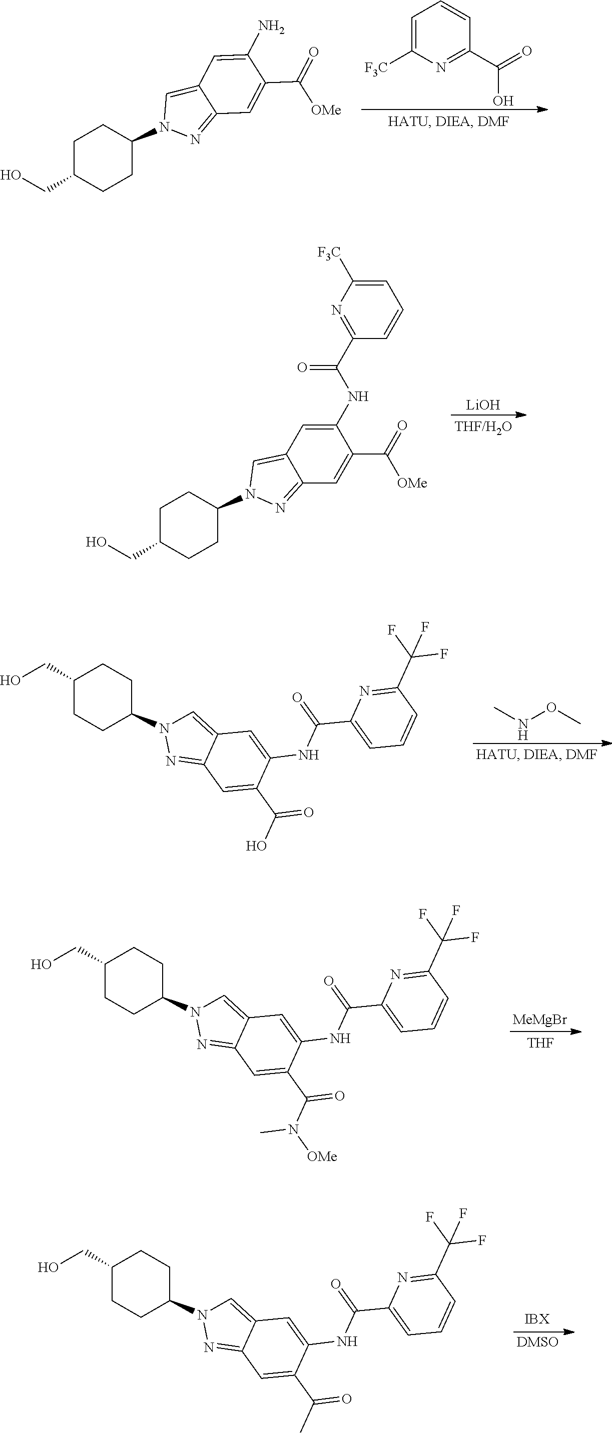

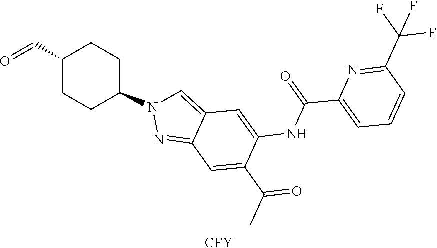 [SMILES: CC(=O)c1cc2nn([C@H]3CC[C@H](C=O)CC3)cc2cc1NC(=O)c1cccc(C(F)(F)F)n1 ; "CFY"]

### Step 1—Methyl 2-[4-(hydroxymethyl)cyclohexyl]-5-[[6-(trifluoromethyl)pyridine-2-carbonyl]amino]indazole-6-carboxylate

To a solution of methyl 5-amino-2-[4-(hydroxymethyl)cyclohexyl]indazole-6-carboxylate (3.50 g, 11.54 mmol, synthesized via Steps 1-3 of Intermediate BXI), 6-(trifluoromethyl)pyridine-2-carboxylic acid (2.09 g, 11.0 mmol, CAS #131747-42-7) and DIEA (4.47 g, 34.6 mmol) in DMF (40 mL) was added HATU (4.83 g, 12.7 mmol). The reaction mixture was stirred at 20° C. for 0.5 hr. On completion, the mixture was quenched with water (200 mL), stirred and filtered. The filter cake was dried in vacuo to give the title compound (5.10 g, 93% yield) as a yellow solid. LC-MS (ESI⁺) m/z 477.2 (M+H)⁺

### Step 2—2-[4-(Hydroxymethyl)cyclohexyl]-5-[[6-(trifluoromethyl)pyridine-2-carbonyl]amino]indazole-6-carboxylic acid

To a solution of methyl 2-[4-(hydroxymethyl)cyclohexyl]-5-[[6-(trifluoromethyl)pyridine-2-carbonyl]amino]indazole-6-carboxylate (1.00 g, 2.10 mmol) in THF (8 mL) and H₂O (2 mL) was added LiOH·H₂O (264 mg, 6.30 mmol). The mixture was then stirred at 25° C. for 2 hrs. On completion, the residue was acidified with HCl (4 M) until the pH=4-5, concentrated in vacuo to give the title compound (970 mg, 99% yield) as white solid. ¹H NMR (400 MHz, DMSO-d₆) δ 13.18 (s, 1H), 9.14 (s, 1H), 8.54 (s, 1H), 8.50-8.41 (m, 2H), 8.37 (t, J=7.6 Hz, 1H), 8.17 (d, J=7.6 Hz, 1H), 4.68-4.39 (m, 2H), 3.28 (d, J=6.0 Hz, 2H), 2.16 (d, J=9.2 Hz, 2H), 1.99-1.83 (m, 4H), 1.49 (d, J=2.8 Hz, 1H), 1.23-1.08 (m, 2H).

### Step 3—2-[4-(Hydroxymethyl)cyclohexyl]-N-methoxy-N-methyl-5-[[6-(trifluoromethyl)pyridine-2-carbonyl]amino]indazole-6-carboxamide

To a solution of 2-[4-(hydroxymethyl)cyclohexyl]-5-[[6-(trifluoromethyl)pyridine-2-carbonyl]amino]indazole-6-carboxylic acid (400 mg, 865 umol) and N-methoxymethanamine (590 mg, 6.06 mmol) in DMF (2 mL) was added DIEA (670 mg, 5.19 mmol). Then, to the reaction was added HATU (361 mg, 951 umol). The mixture was stirred at 25° C. for 16 hrs. On completion, the residue was purified by prep-HPLC(column: Phenomenex Luna C18 200*40 mm*10 um; mobile phase: [water (FA)-ACN]; B %: 31%-61%, 10 min) to give the title compound (220 mg, 50% yield) as white solid. LC-MS (ESI⁺)m/z 506.2 (M+H)⁺.

### Step 4—N-[6-acetyl-2-[4-(hydroxymethyl)cyclohexyl]indazol-5-yl]-6-(trifluoromethyl)pyridine-2-carboxamide

To a solution of 2-[4-(hydroxymethyl)cyclohexyl]-N-methoxy-N-methyl-5-[[6-(trifluoromethyl)pyridine-2-carbonyl]amino]indazole-6-carboxamide (150 mg, 296 umol) in THF (4 mL) was added MeMgBr (3 M, 989 uL) at 0° C. The mixture was stirred at 25° C. for 2 hrs. On completion, the reaction mixture was quenched with NH₄Cl (15 mL). The residue was diluted with water (20 mL) and extracted with EA (25 mL×2). The combined organic layer was washed with brine (25 mL×2), dried over Na₂SO₄ and filtered. The filtrate was concentrated in vacuo. Then, the residue was purified by prep-HPLC (column: Phenomenex luna C18 150*25 mm*10 um; mobile phase: [water (FA)-ACN]; B %: 37%-67%, 10.5 min) to give the title compound (60 mg, 44% yield) as yellow solid. LC-MS (ESI⁺) m/z 461.1 (M+H)⁺.

### Step 5—N-[6-acetyl-2-(4-formylcyclohexyl)indazol-5-yl]-6-(trifluoromethyl)pyridine-2-carboxamide

To a solution of N-[6-acetyl-2-[4-(hydroxymethyl)cyclohexyl]indazol-5-yl]-6-(trifluoromethyl)pyridine-2-carboxamide (50 mg, 108 umol) in DMSO (2 mL) was added IBX (91.2 mg, 325 umol). The mixture was stirred at 50° C. for 1 hr. On completion, the reaction mixture was quenched with saturated Na₂S₂O₃ (5 mL) and saturated NaHCO₃ (5 mL) at 25° C. Then the mixture was stirred for 5 minutes. The mixture was then extracted with EA (20 mL×2) and washed with H₂O (10 mL×2). The reaction mixture was concentrated under reduced pressure to give the title compound (48 mg, 96% yield) as a yellow solid. LC-MS (ESI⁺) m/z 459.1 (M+H)⁺.

### Methyl 2-(4-formylcyclohexyl)-5-[[6-(trifluoromethyl)pyridine-2-carbonyl]amino]indazole-6-carboxylate (Intermediate CFZ)

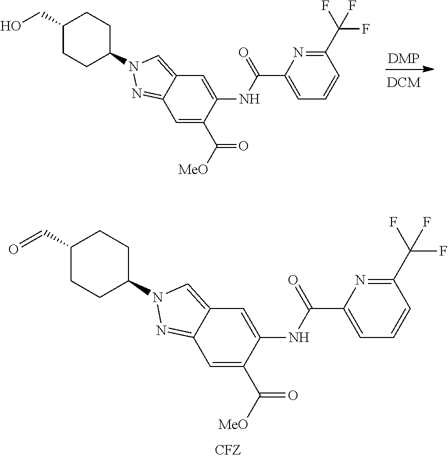 [SMILES: COC(=O)c1cc2nn([C@H]3CC[C@H](CO)CC3)cc2cc1NC(=O)c1cccc(C(F)(F)F)n1 ; COC(=O)c1cc2nn([C@H]3CC[C@H](C=O)CC3)cc2cc1NC(=O)c1cccc(C(F)(F)F)n1]

To a solution of methyl 2-[4-(hydroxymethyl)cyclohexyl]-5-[[6-(trifluoromethyl)pyridine-2-carbonyl]amino]indazole-6-carboxylate (100 mg, 209 umol, synthesized via Step 1 of Intermediate CFY) in DCM (2 mL) and DMF (0.25 mL) was added DMP (133 mg, 314 umol). The reaction was stirred at 25° C. for 16 hrs. On completion, the reaction mixture was quenched with saturated Na₂S₂O₃ solution (10 mL) and saturated NaHCO₃ (10 mL) and stirred for 10 min. The mixture was extracted with DCM (10 mL×2). The combined organic layers were dried over Na₂SO₄, filtered and concentrated in vacuo to give the title compound (90.0 mg, 90% yield) as brown solid. ¹HNMR (400 MHz, DMSO-d₆) δ 12.53 (s, 1H), 9.65 (s, 1H), 9.06 (s, 1H), 8.58 (s, 1H), 8.49-8.44 (m, 2H), 8.39 (t, J=7.6 Hz, 1H), 8.20 (d, J=7.6 Hz, 1H), 4.56 (tt, J=3.6, 11.6 Hz, 1H), 3.96 (s, 3H), 2.45-2.40 (m, 1H), 2.25 (dd, J=2.8, 12.4 Hz, 2H), 2.13 (d, J=11.6 Hz, 2H), 2.06-1.96 (m, 2H), 1.46 (dq, J=3.2, 12.8 Hz, 2H), LC-MS (ESI⁺) m/z 475.0 (M+H)⁺.

### 4-[8-Oxo-6-[6-(trifluoromethyl)-2-pyridyl]pyrazolo[3,4-g][3,1]benzoxazin-2-yl]cyclohexanecarbaldehyde (Intermediate CGA)

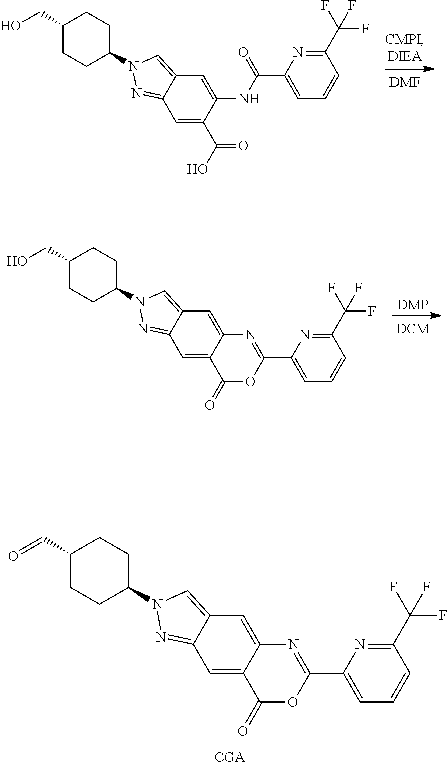

### Step 1—2-[4-(Hydroxymethyl)cyclohexyl]-6-[6-(trifluoromethyl)-2-pyridyl]pyrazolo[3,4-g][3,1]benzoxazin-8-one

To a solution of 2-[4-(hydroxymethyl)cyclohexyl]-5-[[6-(trifluoromethyl)pyridine-2-carbonyl]amino]indazole-6-carboxylic acid (200 mg, 432 umol, synthesized via Steps 1-2 of CFY) in DMF (2 mL) was added CMPI (165 mg, 648 umol) and DIEA (167 mg, 1.30 mmol). The mixture was stirred at 25° C. for 1 hr. On completion, the residue was diluted with water (30 mL) and extracted with EA (20 mL×2). The combined organic layer was dried over Na₂SO₄, filtered and concentrated in vacuo to give the title compound (170 mg, 88% yield) as a white solid. ¹H NMR (400 MHz, DMSO-d₆) δ 8.79 (s, 1H), 8.61-8.55 (m, 2H), 8.33 (t, J=8.0 Hz, 1H), 8.19 (s, 1H), 8.13 (d, J=7.6 Hz, 1H), 4.70-4.55 (m, 1H), 4.50 (t, J=5.2 Hz, 1H), 3.31-3.27 (m, 2H), 2.21 (d, J=10.0 Hz, 2H), 2.03-1.90 (m, 4H), 1.60-1.45 (m, 1H), 1.25-1.15 (m, 2H).

### Step 2—4-[8-Oxo-6-[6-(trifluoromethyl)-2-pyridyl]pyrazolo[3,4-g][3,1]benzoxazin-2-yl]cyclohexanecarbaldehyde

To a solution of 2-[4-(hydroxymethyl)cyclohexyl]-6-[6-(trifluoromethyl)-2-pyridyl]pyrazolo[3,4-g][3,1]benzoxazin-8-one (120 mg, 270 umol) in DCM (2 mL) was added DMP (171 mg, 405 umol). The mixture was stirred at 25° C. for 16 hrs. On completion, the reaction mixture was quenched with saturated Na₂S₂O₃ (5 mL) and saturated NaHCO₃ (5 mL) at 25° C., and stirred for 5 minutes. The mixture was extracted with DCM (20 mL×2) and washed with H₂O (10 mL×2). Then the reaction mixture was concentrated under reduced pressure to give the title compound (110 mg, 92% yield) as yellow solid. LC-MS (ESI⁺) m/z 443.0 (M+H)⁺.

### 1-[8-[4-[(4-aminocyclohexyl)methyl]piperazin-1-yl]-4-isoquinolyl]hexahydropyrimidine-2,4-dione (Intermediate CGB)

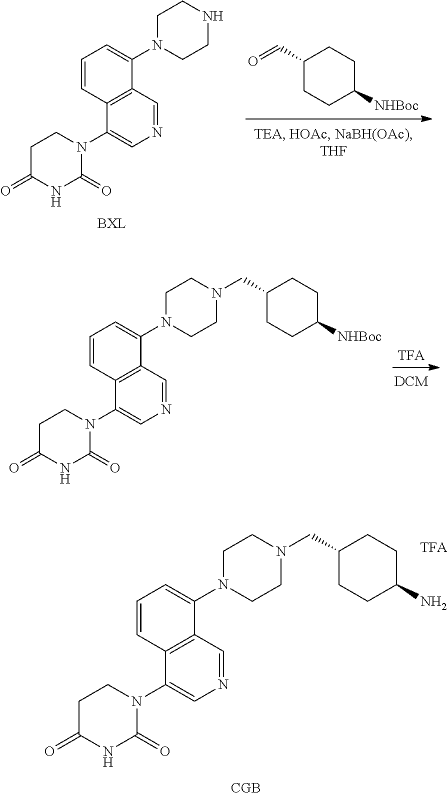

### Step 1—Tert-butyl N-[4-[[4-[4-(2,4-dioxohexahydropyrimidin-1-yl)-8-isoquinolyl]piperazin-1-yl]methyl]cyclohexyl]carbamate

A solution of 1-(8-piperazin-1-yl-4-isoquinolyl)hexahydropyrimidine-2,4-dione (414 mg, 942 umol, TFA, Intermediate BXL), TEA (190 mg, 1.88 mmol), HOAc (169 mg, 2.83 mmol) and tert-butyl N-(4-formylcyclohexyl)carbamate (257 mg, 1.13 mmol, CAS #181308-57-6) in THF (4 mL) was stirred at −10° C. for 0.5 hr. Then, to the above mixture was added NaBH(OAc)₃ (399 mg, 1.88 mmol) and stirred at −10° C. for 2 hrs. On completion, the reaction mixture was quenched by addition water (0.2 mL). The reaction mixture was diluted with EA (100 mL) and washed with water (50 mL). The water phase was extracted with EA (50 mL×3). The combined organic layers were concentrated in vacuo. The residue was purified by prep-HPLC (column: Welch Xtimate C18 250*50 mm*10 um; mobile phase: [water (FA)-ACN]; B %: 2%-30%, 12 min) to give title compound (500 mg, 98% yield) as yellow solid. ¹H NMR (400 MHz, DMSO-d₆) δ 10.52 (s, 1H), 9.43 (s, 1H), 8.54 (s, 1H), 7.77-7.69 (m, 1H), 7.64 (d, J=8.0 Hz, 1H), 7.29 (d, J=7.2 Hz, 1H), 6.79-6.64 (m, 1H), 3.94-3.86 (m, 1H), 3.73-3.66 (m, 1H), 3.27-3.02 (m, 7H), 3.02-2.90 (m, 2H), 2.80-2.70 (m, 2H), 2.67-2.58 (m, 1H), 2.30-2.11 (m, 1H), 1.84-1.76 (m, 4H), 1.60-1.44 (m, 1H), 1.38 (s, 9H), 1.21-1.10 (m, 2H), 0.95 (d, J=6.4 Hz, 2H).

### Step 2—1-[8-[4-[(4-aminocyclohexyl)methyl]piperazin-1-yl]-4-isoquinolyl]hexahydropyrimidine-2,4-dione

To a solution of tert-butyl N-[4-[[4-[4-(2,4-dioxohexahydropyrimidin-1-yl)-8-isoquinolyl]piperazin-1-yl]methyl]cyclohexyl]carbamate (114 mg, 212 umol) in DCM (2 mL) was added TFA (616 mg, 5.40 mmol) at 25° C. The mixture was stirred at 25° C. for 1 hr. On completion, the reaction mixture was concentrated in vacuo to give title compound (116 mg, 100% yield, TFA) as yellow oil. LC-MS (ESI⁺) m/z 437.1 (M+H)⁺.

### [4-[4-[4-(Isopropylamino)-6-(4,4,5,5-tetramethyl-1,3,2-dioxaborolan-2-yl)-3-pyridyl]triazol-1-yl]cyclohexyl]methanol (Intermediate CGC)

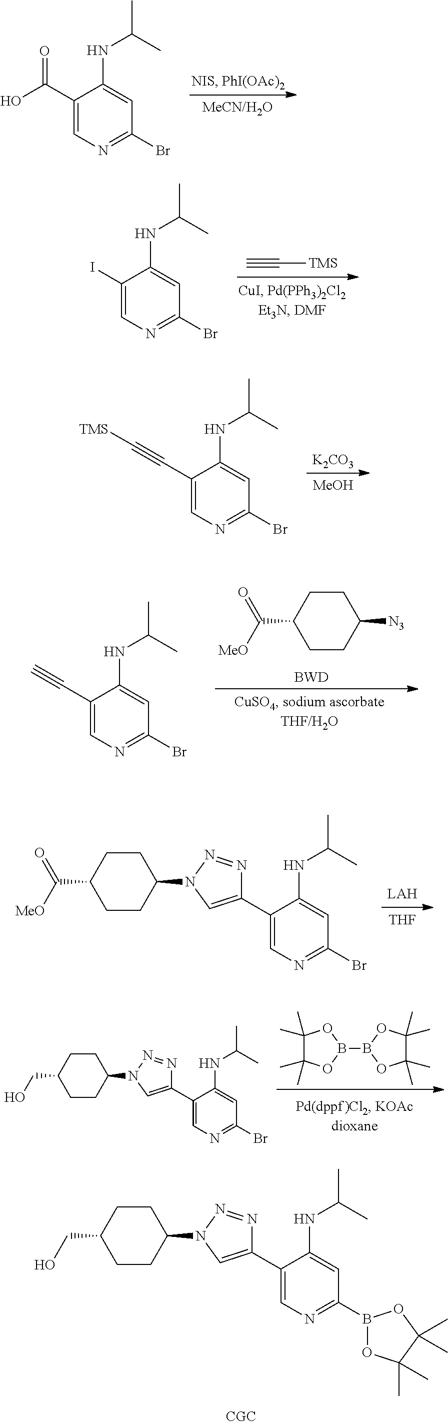

### Step 1—2-Bromo-5-iodo-N-isopropyl-pyridin-4-amine

To a mixture of 6-bromo-4-(isopropylamino)pyridine-3-carboxylic acid (9.40 g, 36.2 mmol, synthesized via Steps 1-3 of Intermediate BVU) in ACN (90 mL) and H₂O (30 mL) was added PhI(OAc)₂ (5.84 g, 18.1 mmol), then the reaction mixture was stirred at 60° C. for 0.5 hr. Next, NIS (8.98 g, 39.9 mmol) was added to the mixture, and the reaction mixture was stirred at 60° C. for 12 hr. On completion, the residue was diluted with water (100 mL) and extracted with EA (2×80 mL). The combined organic layers were dried over Na₂SO₄, filtered and concentrated in vacuo to give a residue. The residue was purified by reversed phase flash (0.1% FA condition) to give the title compound (15.0 g, 60% yield) as a yellow solid. ¹H NMR (400 MHz, DMSO-d₆) δ 8.21 (s, 1H), 6.73 (s, 1H), 5.38 (d, J=8.0 Hz, 1H), 3.91-3.72 (m, 1H), 1.19 (d, J=6.4 Hz, 6H); LC-MS (ESI+) m/z 342.5 (M+H)⁺.

### Step 2—2-Bromo-N-isopropyl-5-(2-trimethylsilylethynyl)pyridin-4-amine

To a solution of 2-bromo-5-iodo-N-isopropyl-pyridin-4-amine (9.00 g, 26.3 mmol) and ethynyl(trimethyl)silane (3.89 g, 39.5 mmol, CAS #1066-54-2) in DMF (50 mL) were added CuI (502 mg, 2.64 mmol), Pd(PPh₃)₂Cl₂ (1.85 g, 2.64 mmol) and TEA (8.01 g, 79.1 mmol). Then the reaction mixture was stirred at 80° C. for 0.5 hr under N₂. On completion, the residue was diluted with water (50 mL) and extracted with EA (2×20 mL). The combined organic layers were washed with brine (2×15 mL) and dried over Na₂SO₄, filtered and the filtrate was concentrated in vacuo to give a residue. The residue was purified by column chromatography (SiO₂, PE:EA=5:1, Rf=0.7) to give the title compound (7.00 g, 77% yield) as a yellow oil. ¹H NMR (400 MHz, DMSO-d₆) δ 7.96 (s, 1H), 6.83 (s, 1H), 5.42 (d, J=7.6 Hz, 1H), 3.83 (qd, J=6.0, 12.8 Hz, 1H), 1.18 (d, J=6.0 Hz, 6H), 0.28-0.16 (m, 9H); LC-MS (ESI+) m/z 312.9 (M+H)⁺.

### Step 3—2-Bromo-5-ethynyl-N-isopropyl-pyridin-4-amine

To a solution of 2-bromo-N-isopropyl-5-(2-trimethylsilylethynyl)pyridin-4-amine (6.00 g, 19.2 mmol) in MeOH (30 mL) was added K₂CO₃ (5.33 g, 38.5 mmol), then the reaction mixture was stirred at 25° C. for 1 hr. On completion, the reaction mixture was filtered and the filtrate was concentrated in vacuo to give a residue. The residue was diluted with water (30 mL) and extracted with EA (2×20 mL). The combined organic layer was washed with brine (15 mL) and dried over Na₂SO₄, filtered and the filtrate was concentrated in vacuo to give the title compound (5.00 g, 98% yield) as a brown oil. ¹H NMR (400 MHz, DMSO-d₆) δ 7.97 (s, 1H), 6.80 (s, 1H), 5.83 (d, J=8.4 Hz, 1H), 4.66 (s, 1H), 3.80 (qd, J=6.4, 14.8 Hz, 1H), 1.17 (d, J=6.4 Hz, 6H); LC-MS (ESI+) m/z 240.7 (M+H)⁺.

### Step 4—Methyl 4-[4-[6-bromo-4-(isopropylamino)-3-pyridyl]triazol-1-yl]cyclohexanecarboxylate

To a solution of 2-bromo-5-ethynyl-N-isopropyl-pyridin-4-amine (2.9 g, 12.1 mmol) and methyl 4-azidocyclohexanecarboxylate (2.44 g, 13.3 mmol, Intermediate BWD) in H₂O (20 mL) and THF (20 mL) was added sodium ascorbate (961 mg, 4.85 mmol) and CuSO₄·5H₂O (1.21 g, 4.85 mmol), then the reaction mixture was stirred at 25° C. for 12 hrs. On completion, the reaction mixture was filtered and concentrated in vacuo. The residue was diluted with water (30 mL) and extracted with EA (2×30 mL). The combined organic layer was washed with brine (30 mL) and dried over Na₂SO₄, filtered and the filtrate was concentrated in vacuo to give a residue. The residue was purified by reversed phase flash (0.10% FA condition) to give the title compound (3 g, 58% yield) as a white solid. ¹H NMR (400 MHz, DMSO-d₆) δ 8.82 (s, 1H), 8.32 (s, 1H), 8.24 (d, J=7.6 Hz, 1H), 6.84 (s, 1H), 4.65-4.50 (m, 1H), 3.91-3.76 (m, 1H), 3.63 (s, 3H), 2.48-2.39 (m, 1H), 2.28-2.17 (m, 2H), 2.08 (dd, J=2.8, 11.2 Hz, 2H), 1.88 (dq, J=3.2, 12.4 Hz, 2H), 1.70-1.53 (m, 2H), 1.23 (d, J=6.4 Hz, 6H); LC-MS (ESI+) m/z 422.3 (M+H)⁺.

### Step 5—[4-[4-[6-Bromo-4-(isopropylamino)-3-pyridyl]triazol-1-yl]cyclohexyl]methanol

To a mixture of methyl 4-[4-[6-bromo-4-(isopropylamino)-3-pyridyl]triazol-1-yl]cyclohexanecarboxylate (1.90 g, 4.50 mmol) in THF (30 mL) was added LAH (221 mg, 5.85 mmol) at 0° C. The mixture was then stirred at 0° C. for 0.5 hr. On completion, the reaction mixture was quenched with H₂O (0.1 mL) and 15% NaOH (0.6 mL) at 20° C., then dried over anhydrous sodium sulfate, filtered and concentrated under reduced pressure to give a residue. The residue was purified by reversed phase flash (0.1% FA condition) to give the title compound (2.40 g, 98% yield) as a white solid. ¹H NMR (400 MHz, DMSO-d₆) δ 8.84 (s, 1H), 8.32 (s, 1H), 8.27 (d, J=7.6 Hz, 1H), 6.84 (s, 1H), 4.56-4.44 (m, 2H), 3.83 (qd, J=6.4, 13.2 Hz, 1H), 3.28 (t, J=5.6 Hz, 2H), 2.18 (d, J=9.6 Hz, 2H), 1.90 (d, J=11.6 Hz, 2H), 1.86-1.72 (m, 2H), 1.55-1.38 (m, 1H), 1.23 (d, J=6.4 Hz, 6H), 1.21-1.07 (m, 2H); LC-MS (ESI+) m/z 396.1 (M+H)⁺.

### Step 6-[4-[4-[4-(Isopropylamino)-6-(4,4,5,5-tetramethyl-1,3,2-dioxaborolan-2-yl)-3-pyridyl] triazol-1-yl]cyclohexyl]methanol

To a solution of [4-[4-[6-bromo-4-(isopropylamino)-3-pyridyl]triazol-1-yl]cyclohexyl]methanol (100 mg, 253 umol) and 4,4,5,5-tetramethyl-2-(4,4,5,5-tetramethyl-1,3,2-dioxaborolan-2-yl)-1,3,2-dioxaborolane (128 mg, 507 umol, CAS #73183-34-3) in dioxane (4 mL) were added KOAc (74.6 mg, 760 umol) and Pd(dppf)Cl₂·CH₂Cl₂ (20.7 mg, 25.3 umol). The mixture was stirred at 100° C. for 3 hrs under N₂. On completion, the crude compound was used without work-up and further purification to give the title compound (447 mg, 99% yield) as a frey solid. LC-MS (ESI+) m/z 442.2 (M+H)⁺.

### 7-[5-[1-(4-Formylcyclohexyl)triazol-4-yl]-4-(isopropylamino)-2-pyridyl]pyrrolo [1,2-b]pyridazine-3-carbonitrile (Intermediate CGD)

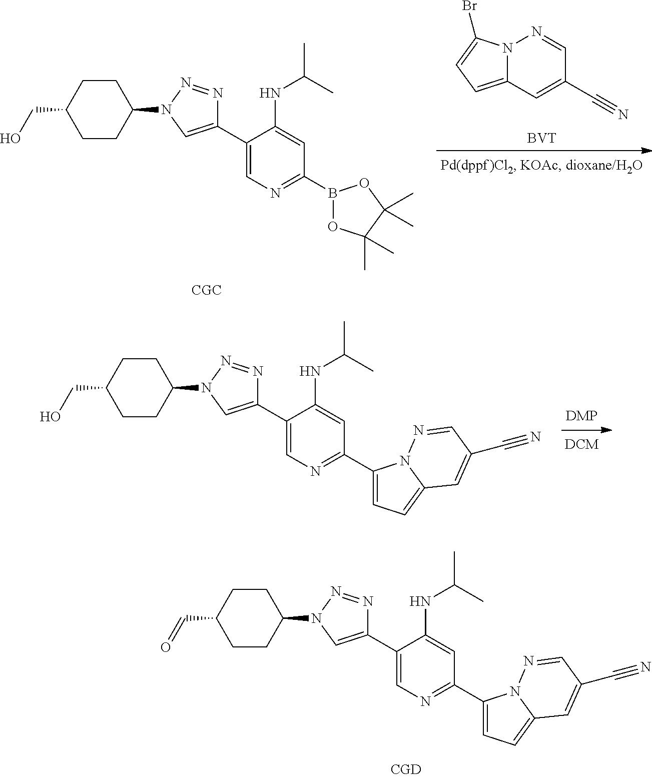

### Step 1—7-[5-[1-[4-(Hydroxymethyl)cyclohexyl]triazol-4-yl]-4-(isopropylamino)-2-pyridyl]pyrrolo [1,2-b]pyridazine-3-carbonitrile

To a solution of [4-[4-[4-(isopropylamino)-6-(4,4,5,5-tetramethyl-1,3,2-dioxaborolan-2-yl)-3-pyridyl]triazol-1-yl]cyclohexyl]methanol (111 mg, 251 umol, Intermediate CGC) and 7-bromopyrrolo[1,2-b]pyridazine-3-carbonitrile (44.6 mg, 201 umol, Intermediate BVT) in dioxane (4 mL) and H₂O (0.4 mL) were added KOAc (24.6 mg, 251 umol) and Pd(dppf)Cl₂·CH₂Cl₂ (20.5 mg, 25.1 umol). The mixture was then stirred at 100° C. for 2 hrs under N₂. On completion, the reaction mixture was diluted with EA (60 mL), filtered and concentrated in vacuo. The residue was purified by reversed phase flash (0.1% FA condition), then purified by prep-TLC (SiO₂, DCM:MeOH=10:1) to give the title compound (59.0 mg, 12% yield) as a yellow solid. ¹H NMR (400 MHz, DMSO-d₆) δ 8.85 (s, 1H), 8.81 (d, J=2.0 Hz, 1H), 8.72-8.65 (m, 2H), 8.18 (d, J=7.2 Hz, 1H), 8.13 (s, 1H), 7.81 (d, J=4.8 Hz, 1H), 7.10 (d, J=4.8 Hz, 1H), 4.60-4.47 (m, 2H), 3.89 (qd, J=6.4, 12.8 Hz, 1H), 3.30-3.27 (m, 2H), 2.21 (d, J=11.6 Hz, 2H), 1.98-1.77 (m, 4H), 1.56-1.41 (m, 1H), 1.34 (d, J=6.4 Hz, 6H), 1.25-1.10 (m, 2H); LC-MS (ESI+) m/z 457.6 (M+H)⁺.

### Step 2—7-[5-[1-(4-Formylcyclohexyl)triazol-4-yl]-4-(isopropylamino)-2-pyridyl]pyrrolo [1,2-b]pyridazine-3-carbonitrile

To a mixture of 7-[5-[1-[4-(hydroxymethyl)cyclohexyl]triazol-4-yl]-4-(isopropylamino)-2-pyridyl]pyrrolo[1,2-b]pyridazine-3-carbonitrile (49.0 mg, 107 umol) in DCM (1.5 mL) was added DMP (68.2 mg, 160 umol). The mixture then was stirred at 25° C. for 1 hr. On completion, the reaction mixture was quenched by saturated NaHCO₃ (2.5 mL) and Na₂S₂O₃ (2.5 mL), then the mixture was extracted with DCM (3×5 mL). The combined organic layers were washed with NaCl (5 mL×2), dried over Na₂SO₄, filtered and concentrated in vacuo to give the title compound (57.0 mg, 99% yield) as a yellow solid. LC-MS (ESI+) m/z 455.1 (M+H)⁺.

### 1-(8-bromo-6-fluoro-4-isoquinolyl)-3-[(4-methoxyphenyl)methyl]hexahydropyrimidine-2,4-dione (Intermediate CGE)

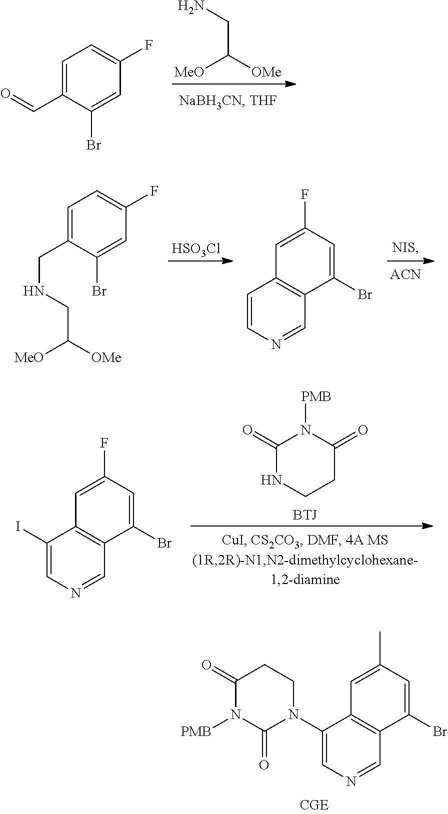

### Step 1—N-[(2-bromo-4-fluoro-phenyl)methyl]-2,2-dimethoxy-ethanamine

To a solution of 2-bromo-4-fluoro-benzaldehyde (5 g, 24.6 mmol, CAS #59142-68-6) and 2,2-dimethoxyethanamine (2.85 g, 27.0 mmol, CAS #645-36-3) in MeOH (60 mL) was added NaBH₃CN (3.10 g, 49.2 mmol), then the reaction was stirred at 25° C. for 2 hrs. On completion, the reaction was quenched with water (5 mL) and the reaction was diluted with EA (80 mL), and washed water (30 mL×4). The organic layer was separated and dried over Na₂SO₄, filtered and concentrated in vacuo. The residue was purified by column chromatography (SiO₂, PE:EA=5:1 to 3:1) to give the title compound (3.2 g, 44% yield ) as yellow oil. ¹H NMR (400 MHz, DMSO-d₆) δ 7.59-7.46 (m, 2H), 7.25-7.20 (m, 1H), 4.40 (t, J=5.6 Hz, 1H), 3.74 (s, 2H), 3.25 (s, 6H), 2.59 (d, J=5.6 Hz, 2H), 2.01 (s, 1H).

### Step 2—8-Bromo-6-fluoro-isoquinoline

To a solution of HSO₃Cl (3.99 g, 34.2 mmol, 2.28 mL) was added N-[(2-bromo-4-fluoro-phenyl)methyl]-2,2-dimethoxy-ethanamine (1.00 g, 3.42 mmol) slowly at −10° C., then the reaction mixture was stirred at 90° C. for 1 hr. On completion, the reaction mixture was added into ice water (40 mL), and extracted with DCM (80 mL). To the aqueous phase was add NaOH (1 N) until the pH=8, then extracted with DCM (100 mL). The organic layer was washed with water (30 mL×4), dried over Na₂SO₄, filtered and concentrated in vacuo to give the title compound (449 mg, 58% yield) as brown oil. ¹H NMR (400 MHz, DMSO-d₆) δ 9.45 (s, 1H), 8.64 (d, J=5.6 Hz, 1H), 8.09 (dd, J=2.4, 8.8 Hz, 1H), 7.94-7.87 (m, 2H).

### Step 3—8-bromo-6-fluoro-4-iodo-isoquinoline

To a solution of 8-bromo-6-fluoro-isoquinoline (349 mg, 1.54 mmol) in AcOH (4 mL) was added NIS (416 mg, 1.85 mmol), then the reaction mixture was stirred at 50° C. for 10 hrs. On completion, the residue was diluted with EA (20 mL), and concentrated in vacuo. The residue was purified by column chromatography (SiO₂, PE:PA=10:1) to give the title compound (158 mg, 29% yield) as a white solid. LC-MS (ESI⁺) m/z 351.8 (M+H)⁺.

### Step 4—1-(8-bromo-6-fluoro-4-isoquinolyl)-3-[(4-methoxyphenyl)methyl]hexahydropyrimidine-2,4-dione

To a solution of 8-bromo-6-fluoro-4-iodo-isoquinoline (20 mg, 56.8 umol) and 3-[(4-methoxyphenyl)methyl]hexahydropyrimidine-2,4-dione (14.6 mg, 62.5 umol, Intermediate BTJ) in DMF (2 mL) was added Cs₂CO₃ (37.0 mg, 113 umol), CuI (4.33 mg, 22.7 umol), (1R,2R)-N1,N2-dimethylcyclohexane-1,2-diamine (3.23 mg, 22.73 umol) and 4 Å molecular sieves. Then the reaction mixture was stirred at 70° C. for 16 hrs under N₂. On completion, the mixture was concentrated in vacuo. The crude product was purified by reversed-phase HPLC (column: Phenomenex luna C18 150*25 mm*10 um; mobile phase: [water (FA)-ACN]; B %: 39%-69%, 8 min) to give the title compound (4 mg, 15% yield) as a white solid. LC-MS (ESI⁺) m/z 459.7 (M+H)⁺.

### 1-(6-fluoro-8-piperazin-1-yl-4-isoquinolyl)hexahydropyrimidine-2,4-dione (Intermediate CGF)

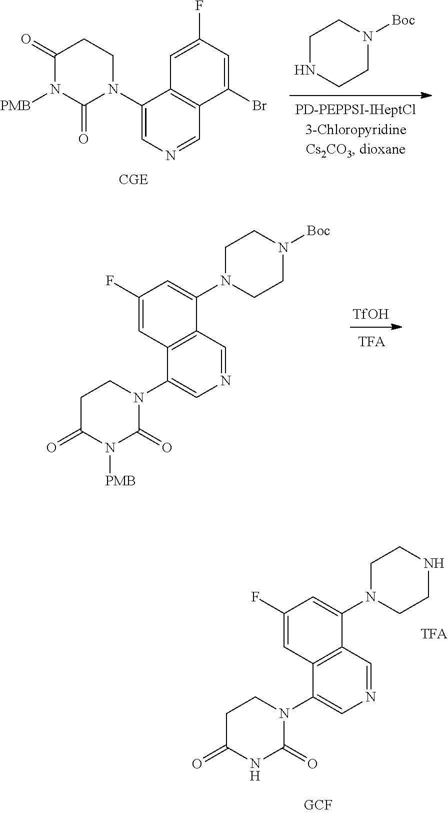

### Step 1—Tert-butyl 4-[6-fluoro-4-[3-[(4-methoxyphenyl)methyl]-2,4-dioxo-hexahydropyrimidin-1-yl]-8-isoquinolyl]piperazine-1-carboxylate

To a solution of 1-(8-bromo-6-fluoro-4-isoquinolyl)-3-[(4-methoxyphenyl)methyl]hexahydropyrimidine-2,4-dione (100 mg, 218 umol, Intermediate CGE) and tert-butyl piperazine-1-carboxylate (64.5 mg, 261 umol, HOAc, CAS #143238-38-4) in dioxane (3 mL) was added Cs₂CO₃ (213 mg, 654 umol) and PD-PEPPSI-IHeptCl 3-Chloropyridine (20.2 mg, 21.8 umol). The reaction was stirred at 100° C. for 16 hours under N₂. On completion, the reaction mixture was filtered and concentrated in vacuo. The residue was purified by prep-TLC (SiO₂, PE:EA=1:2, P1:Rf=0.5) to give the title compound (120 mg, 97% yield) as yellow solid. ¹H NMR (400 MHz, DMSO-d₆) δ 9.40 (s, 1H), 8.56 (s, 1H), 7.35 (dd, J=1.6, 9.6 Hz, 1H), 7.26 (d, J=8.4 Hz, 2H), 7.17 (dd, J=2.0, 11.2 Hz, 1H), 6.87 (d, J=8.4 Hz, 2H), 4.90-4.76 (m, 2H), 3.95-3.86 (m, 1H), 3.78-3.69 (m, 4H), 3.64 (s, 3H), 3.26-3.03 (m, 6H), 2.93 (m, 1H), 1.44 (s, 9H).

### Step 2—1-(6-fluoro-8-piperazin-1-yl-4-isoquinolyl)hexahydropyrimidine-2,4-dione

To a solution of tert-butyl 4-[6-fluoro-4-[3-[(4-methoxyphenyl)methyl]-2,4-dioxo-hexahydropyrimidin-1-yl]-8-isoquinolyl]piperazine-1-carboxylate (50.0 mg, 88.7 umol,) in TFA (0.3 mL) was added TfOH (340 mg, 2.27 mmol), then the reaction was stirred at 70° C. for 0.5 hour. On completion, the reaction mixture was concentrated in vacuo to give the title compound (40 mg, 98% yield, TFA) as brown oil. LC-MS (ESI⁺) m/z 344.0 (M+H)⁺.

### 3-[4-(3,6-Diazabicyclo[3.1.1]heptan-6-yl)-3-methyl-2-oxo-benzimidazol-1-yl]piperidine-2,6-dione (Intermediate CGG)

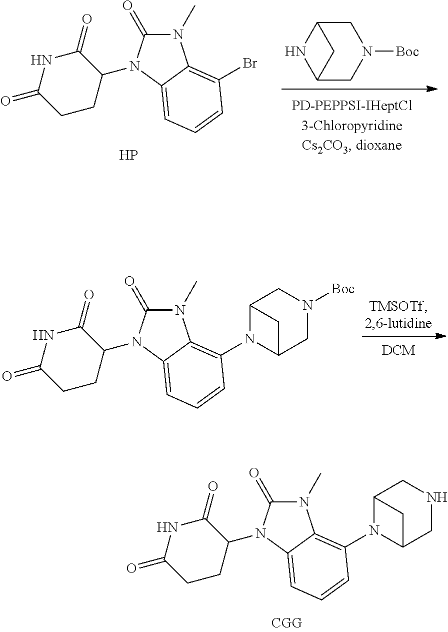

### Step 1—Tert-butyl 6-[1-(2,6-dioxo-3-piperidyl)-3-methyl-2-oxo-benzimidazol-4-yl]-3,6-diazabicyclo [3.1.1]heptane-3-carboxylate

To a mixture of 3-(4-bromo-3-methyl-2-oxo-benzimidazol-1-yl)piperidine-2,6-dione (100 mg, 295 umol, Intermediate HP) in dioxane (5 mL) was tert-butyl 3,6-diazabicyclo[3.1.1]heptane-3-carboxylate (70.3 mg, 354 umol, CAS #1251017-66-9), Cs₂CO₃ (289 mg, 887 umol) and PD-PEPPSI-IHeptCl 3-Chloropyridine (14.3 mg, 14.7 umol). Then the reaction mixture was stirred at 100° C. for 48 hrs. On completion, the reaction mixture was filtered and concentrated in vacuo. The residue was purified by reverse phase (0.1% FA condition) to give the title compound (200 mg, 29% yield) as white solid. LC-MS (ESI⁺) m/z 456.1 (M+H)⁺.

### Step 2—3-[4-(3,6-Diazabicyclo[3.1.1]heptan-6-yl)-3-methyl-2-oxo-benzimidazol-1-yl]piperidine-2,6-dione

To a solution of tert-butyl 6-[1-(2,6-dioxo-3-piperidyl)-3-methyl-2-oxo-benzimidazol-4-yl]-3,6-diaza bicyclo[3.1.1]heptane-3-carboxylate (40 mg, 87.8 umol) in DCM (2 mL) were added 2,6-lutidine (18.8 mg, 175 umol, 20.4 uL) and TMSOTf (29.2 mg, 131 umol, 23.8 uL) at 0° C. Then the reaction mixture was stirred for 5 hr at 25° C. On completion, the reaction mixture was concentrated in vacuo. The residue was purified by prep-HPLC (column: Phenomenex luna C18 150*25 mm*10 um; mobile phase: [water(FA)-ACN]; B %: 1%-22%, 10.5 min) to give the title compound (12.0 mg, 38% yield) as white solid. LC-MS (ESI⁺) m/z 356.3 (M+H)⁺.

### 4-(Isopropylamino)-2-thiazol-5-yl-thieno[2,3-b]pyridine-5-carboxylic acid (Intermediate CGH)

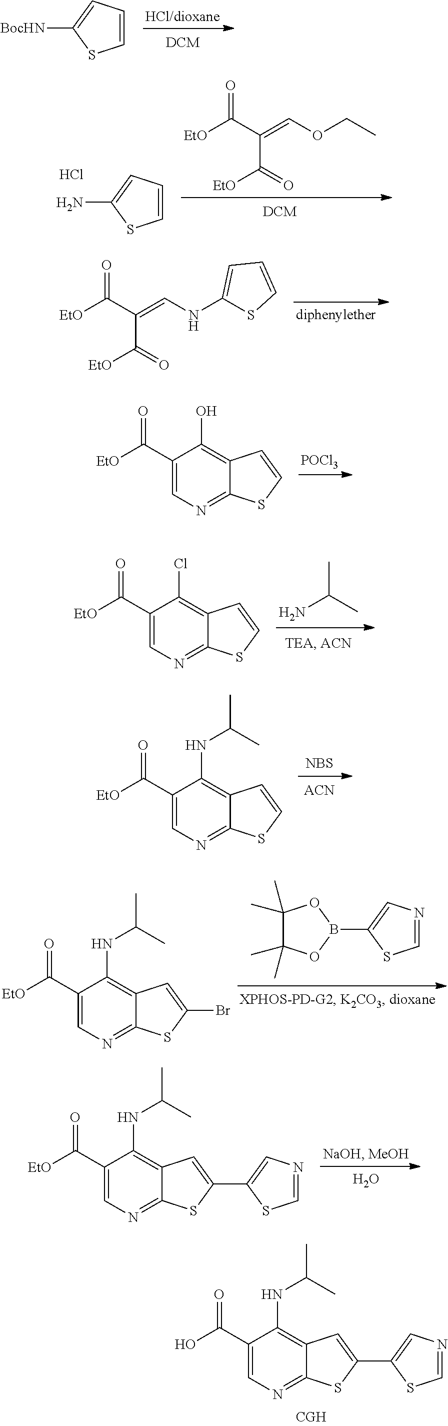

### Step 1—Thiophen-2-amine

To a mixture of tert-butyl N-(2-thienyl)carbamate (20.0 g, 100 mmol, CAS #56267-50-6) in DCM (80 mL) was added HCl/dioxane (4 M, 70 mL). The reaction mixture was stirred at 25° C. for 6 hr. On completion, the reaction mixture was concentrated in vacuo. The residue was diluted with NH₃·H₂O (30 mL) and H₂O (200 ml) and extracted with DCM (2×300 mL). The combined organic layer was dried over Na₂SO₄, filtered and concentrated in vacuo to give the compound (7.00 g, 70% yield) as brown solid.

### Step 2—Diethyl 2-[(2-thienylamino)methylene]propanedioate

A mixture of thiophen-2-amine (7.00 g, 70.6 mmol) and diethyl 2-(ethoxymethylene)propanedioate (15.2 g, 70.6 mmol) in DCM (50 mL) was stirred at 25° C. for 12 hour. On completion, the reaction mixture was concentrated in vacuo. The residue was purified by column chromatography to give the title compound (6.10 g, 32% yield) as yellow oil. ¹H NMR (400 MHz, CDCl₃) δ 11.12 (d, J=12.8 Hz, 1H), 8.24 (d, J=13.2 Hz, 1H), 6.92-6.83 (m, 2H), 6.70-6.63 (m, 1H), 4.34-4.27 (m, 2H), 4.26-4.20 (m, 2H), 1.38 (t, J=7.2 Hz, 3H), 1.32 (t, J=7.2 Hz, 3H). LC-MS (ESI⁺) m/z 270.0 (M+H)⁺.

### Step 3—Ethyl 4-hydroxythieno[2,3-b]pyridine-5-carboxylate

A mixture of diethyl 2-[(2-thienylamino)methylene]propanedioate (5.60 g, 20.7 mmol) in diphenylether (30 mL) was stirred at 230° C. for 5 hour. On completion, the reaction mixture was concentrated in vacuo. The residue was purified by column chromatography to give the title compound (2.90 g, 62% yield) as yellow solid. ¹H NMR (400 MHz, CDCl₃) δ 11.12 (d, J=13.2 Hz, 1H), 8.25 (d, J=13.2 Hz, 1H), 6.90-6.87 (m, 1H), 6.74-6.64 (dd, 1H), 4.35-4.27 (m, 2H), 1.38 (t, J=7.2 Hz, 3H).

### Step 4—Ethyl 4-chlorothieno[2,3-b]pyridine-5-carboxylate

To a mixture of ethyl 4-hydroxythieno[2,3-b]pyridine-5-carboxylate (2.60 g, 11.6 mmol) in dioxane (20 mL) was added POCl₃ (10.7 g, 69.8 mmol). The reaction mixture was stirred at 105° C. for 2 hour. On completion, the residue was purified by column chromatography to give the title compound (2.80 g, 99% yield) as brown. ¹H NMR (400 MHz, DMSO-d₆) δ 8.91 (s, 1H), 8.13 (d, J=6.0 Hz, 1H), 7.59 (d, J=6.0 Hz, 1H), 4.43-4.32 (m, 2H), 1.35 (t, J=7.2 Hz, 3H), LC-MS (ESI⁺) m/z 242.0 (M+H)⁺.

### Step 5—Ethyl 4-(isopropylamino)thieno[2,3-b]pyridine-5-carboxylate

To a mixture of ethyl 4-chlorothieno[2,3-b]pyridine-5-carboxylate (2.80 g, 11.5 mmol) and propan-2-amine (3.42 g, 57.9 mmol) in ACN (30 mL) was added TEA (5.86 g, 57.9 mmol). The reaction mixture was stirred at 90° C. for 6 hr. On completion, the residue was diluted with water (60 mL) and extracted with EA (2×60 mL). The combined organic layer was dried over Na₂SO₄, filtered and concentrated in vacuo to give the title compound (2.80 g, 91% yield) as brown solid. LC-MS (ESI⁺) m/z 265.2 (M+H)⁺.

### Step 6—Ethyl 2-bromo-4-(isopropylamino)thieno[2,3-b]pyridine-5-carboxylate

To a mixture of ethyl 4-(isopropylamino)thieno[2,3-b]pyridine-5-carboxylate (2.80 g, 10.5 mmol) in ACN (30 mL) was added NBS (2.83 g, 15.8 mmol). The reaction mixture was stirred at 25° C. for 6 hr. On completion, the residue was diluted with water (50 mL) and extracted with EA (2×60 mL). The combined organic layer was dried over Na₂SO₄, filtered and concentrated in vacuo to give a residue. The residue was purified by reverse phase (0.1% FA condition) to give the title compound (2.02 g, 550% yield) as brown solid. ¹H NMR (400 MHz, DMSO-d₆) δ 9.15-8.61 (m, 1H), 7.78 (s, 1H), 4.40-4.22 (m, 2H), 3.35 (s, 5H), 1.37-1.23 (m, 6H). LC-MS (ESI⁺) m/z 344.9 (M+H)⁺.

### Step 7—Ethyl 4-(isopropylamino)-2-thiazol-5-yl-thieno[2,3-b]pyridine-5-carboxylate

To a mixture of ethyl 2-bromo-4-(isopropylamino)thieno[2,3-b]pyridine-5-carboxylate (1.97 g, 5.74 mmol) and 5-(4,4,5,5-tetramethyl-1,3,2-dioxaborolan-2-yl)thiazole (4.85 g, 22.9 mmol) in dioxane (20 mL) was added XPHOS-PD-G2 (451 mg, 573 umol) and K₂CO₃ (2.38 g, 17.2 mmol). The reaction mixture was then stirred at 90° C. for 12 hour. On completion, the residue was diluted with water (50 mL) and extracted with EA (2×50 mL). The combined organic layer was dried over Na₂SO₄, filtered and concentrated in vacuo to give a residue. The residue was purified by reverse phase (0.1% FA condition) to give the title compound (1.80 g, 90% yield) as brown solid. LC-MS (ESI⁺) m/z 348.5 (M+H)⁺.

### Step 8—4-(Isopropylamino)-2-thiazol-5-yl-thieno[2,3-b]pyridine-5-carboxylic acid

To a mixture of ethyl 4-(isopropylamino)-2-thiazol-5-yl-thieno[2,3-b]pyridine-5-carboxylate (1.30 g, 3.74 mmol) in MeOH (12 mL) and H₂O (3 mL) was added NaOH (299 mg, 7.48 mmol). Then the reaction mixture was stirred at 60° C. for 2 hour. On completion, the residue was diluted with water (30 mL) and extracted with EA (2×30 mL). The combined organic layer was dried over Na₂SO₄, filtered and concentrated in vacuo to give a residue. The reaction mixture was diluted with water (20 mL) and acidified with citric acid until the pH=3-4. A solid precipitation formed which was then filtered and dried in vacuo to give the title compound (950 mg, 79% yield) as brown solid. ¹H NMR (400 MHz, DMSO-d₆) δ 9.43-9.34 (d, 1H), 9.14 (s, 1H), 8.70 (s, 1H), 8.32 (s, 1H), 7.81 (s, 1H), 4.52-4.36 (m, 1H), 1.32 (d, J=6.0 Hz, 6H). LC-MS (ESI⁺) m/z 319.9 (M+H)⁺.

### 4-[4-[4-(Isopropylamino)-2-thiazol-5-yl-thieno[2,3-b]pyridin-5-yl]triazol-1-yl]cyclohexanecarbaldehyde (Intermediate CGI)

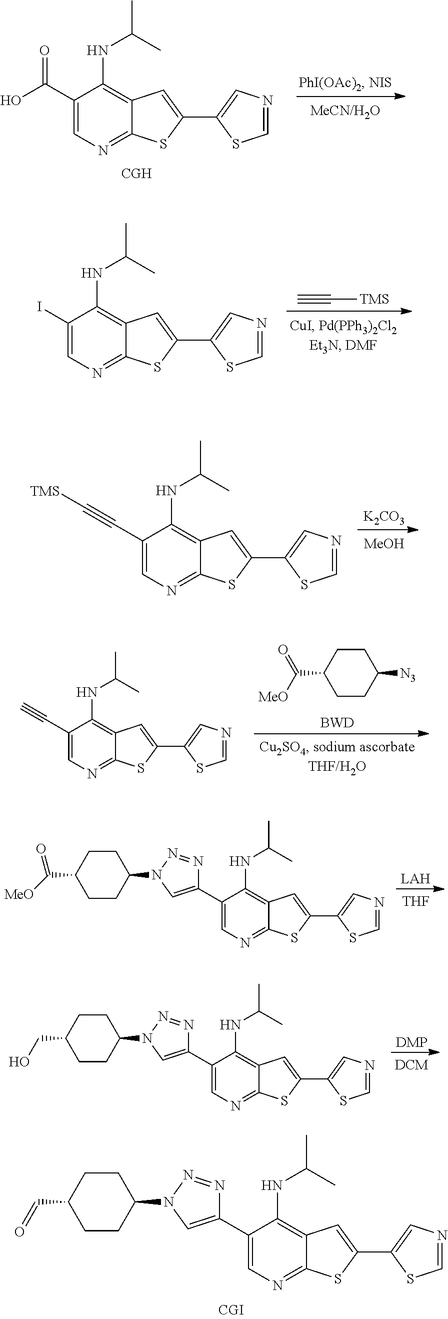

### Step 1—5-Iodo-N-isopropyl-2-thiazol-5-yl-thieno[2,3-b]pyridin-4-amine

To a mixture of 4-(isopropylamino)-2-thiazol-5-yl-thieno[2,3-b]pyridine-5-carboxylic acid (900 mg, 2.82 mmol, Intermediate CGH) in MeCN (12 mL) and H₂O (4 mL) was added PhI(OAc)₂ (453 mg, 1.41 mmol). The reaction mixture was stirred at 60° C. for 0.5 hour, then NIS (697 mg, 3.10 mmol) was added to the mixture and the reaction mixture was stirred at 60° C. for 12 hr. On completion, the residue was diluted with water (30 mL) and extracted with EA (2×30 mL). The combined organic layer was dried over Na₂SO₄, filtered and concentrated in vacuo to give a residue. The residue was purified by reverse phase (0.1% FA condition) to give title compound (1.00 g, 88% yield) as brown solid. LC-MS (ESI⁺) m/z 401.8 (M+H)⁺.

### Step 2—N-isopropyl-2-thiazol-5-yl-5-(2-trimethylsilylethynyl)thieno[2,3-b]pyridin-4-amine

To a mixture of 5-iodo-N-isopropyl-2-thiazol-5-yl-thieno[2,3-b]pyridin-4-amine (1.00 g, 2.49 mmol) and ethynyl(trimethyl)silane (1.22 g, 12.4 mmol) in DMF (20 mL) was added Pd(PPh₃)₂Cl₂ (174 mg, 249 umol), CuI (47.4 mg, 249 umol) and Et₃N (1.26 g, 12.4 mmol). The reaction mixture was then stirred at 80° C. for 16 hours. On completion, the residue was diluted with water (30 mL) and extracted with EA (2×30 mL). The combined organic layer was dried over Na₂SO₄, filtered and concentrated in vacuo to give a residue. The residue was purified by column chromatography to give the title compound (400 mg, 43% yield) as brown oil. ¹H NMR (400 MHz, CDCl₃) δ 8.78 (s, 1H), 8.26 (s, 1H), 8.05 (s, 1H), 7.45 (s, 1H), 2.97-2.88 (m, 2H), 1.40 (d, J=6.4 Hz, 6H), 0.34-0.28 (m, 9H).

### Step 3—5-Ethynyl-N-isopropyl-2-thiazol-5-yl-thieno[2,3-b]pyridin-4-amine

To a mixture of N-isopropyl-2-thiazol-5-yl-5-(2-trimethylsilylethynyl)thieno[2,3-b]pyridin-4-amine (200 mg, 538 umol) in MeOH (2 mL) was added K₂CO₃ (148 mg, 1.08 mmol). The reaction mixture was then stirred at 25° C. for 1 hr. On completion, the residue was diluted with water (30 mL) and extracted with EA (2×20 mL). The combined organic layer was dried over Na₂SO₄, filtered and concentrated in vacuo to give the title compound (160 mg, 99% yield) as brown solid. LC-MS (ESI⁺) m/z 300.0 (M+H)⁺.

### Step 4—Methyl 4-[4-[4-(isopropylamino)-2-thiazol-5-yl-thieno[2,3-b]pyridin-5-yl]triazol-1-yl] cyclohexanecarboxylate

To a mixture of methyl 4-azidocyclohexanecarboxylate (85.6 mg, 467 umol, Intermediate BWD) and 5-ethynyl-N-isopropyl-2-thiazol-5-yl-thieno[2,3-b]pyridin-4-amine (140 mg, 467 umol) in THF (1 mL) and H₂O (1 mL) was added CuSO₄ (29.8 mg, 187 umol) and sodium ascorbate (37.0 mg, 187 umol). The reaction mixture was then stirred at 25° C. for 1 hour. On completion, the residue was diluted with water (30 mL) and extracted with EA (2×20 mL). The combined organic layer was dried over Na₂SO₄, filtered and concentrated in vacuo to give a residue. The residue was purified by reverse phase (0.1% FA condition) to give the title compound (170 mg, 75% yield) as brown solid. ¹H NMR (400 MHz, DMSO-d₆) δ 9.14 (s, 1H), 8.86-8.76 (m, 2H), 8.51 (s, 1H), 8.30 (s, 1H), 7.85 (s, 1H), 4.66-4.54 (m, 1H), 4.45-4.32 (m, 1H), 3.64 (s, 3H), 2.27-2.21 (m, 2H), 2.13-2.04 (m, 3H), 1.96-1.87 (m, 2H), 1.67-1.55 (m, 2H), 1.31 (d, J=6.0 Hz, 6H). LC-MS (ESI⁺) m/z 483.2 (M+H)⁺.

### Step 5—[4-[4-[4-(Isopropylamino)-2-thiazol-5-yl-thieno[2,3-b]pyridin-5-yl]triazol-1-yl]cyclohexyl] methanol

To a mixture of methyl 4-[4-[4-(isopropylamino)-2-thiazol-5-yl-thieno[2,3-b]pyridin-5-yl]triazol-1-yl]cyclohexanecarboxylate (60.0 mg, 124 umol) in THF (1 mL) was added LiAlH₄ (5.19 mg, 136 umol) at 0° C. The reaction mixture was then stirred at 0° C. for 0.5 hour. On completion, the reaction mixture was quenched with water (0.1 mL), then to the mixture was added NaOH (15%) 0.2 ml. Next, DCM(3 mL) was added, then the mixture was filtered and concentrated in vacuo to give the title compound (50.0 mg, 88% yield) as yellow solid. LC-MS (ESI⁺) m/z 455.2 (M+H)⁺.

### Step 6—4-[4-[4-(Isopropylamino)-2-thiazol-5-yl-thieno[2,3-b]pyridin-5-yl]triazol-1-yl]cyclohexanecarbaldehyde

To a mixture of [4-[4-[4-(isopropylamino)-2-thiazol-5-yl-thieno[2,3-b]pyridin-5-yl]triazol-1-yl]cyclohexyl]methanol (50.0 mg, 109 umol) in DCM (1 mL) was added DMP (60.6 mg, 142 umol). The reaction mixture was then stirred at 25° C. for 1 hour. On completion, the reaction mixture was quenched with Na₂S₂O₃ (8 mL) and saturated NaHCO₃ (8 mL) at 25° C., then stirred for 20 minutes. The mixture was then extracted with DCM (2×20 mL) and the organic layers were separated and concentrated in vacuo to give the title compound (45.0 mg, 90% yield) as yellow solid. LC-MS (ESI⁺) m/z 453.1 (M+H)⁺.

### Benzyl 4-[(3-bromo-1-bicyclo[1.1.1]pentanyl)methoxy]piperidine-1-carboxylate (Intermediate CGJ)

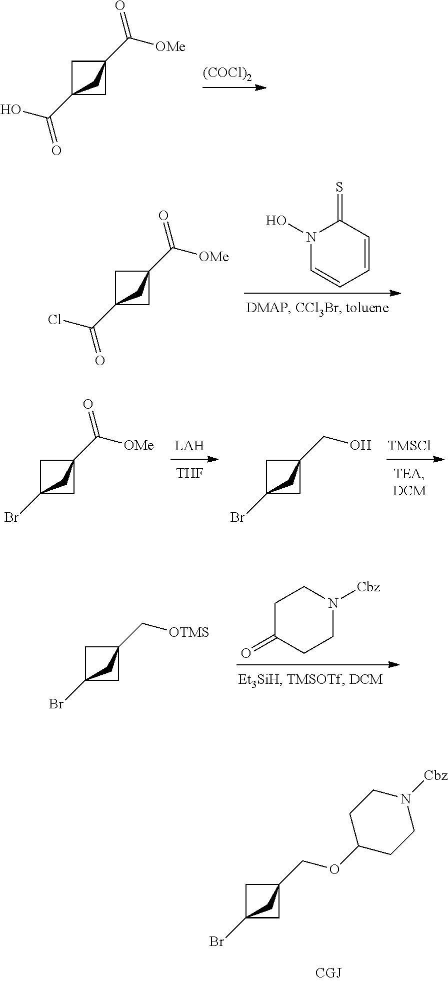

### Step 1—Methyl 3-chlorocarbonylbicyclo[1.1.1]pentane-1-carboxylate

To a solution of 3-methoxycarbonylbicyclo[1.1.1]pentane-1-carboxylic acid (14.0 g, 82.2 mmol, CAS #83249-10-9) was dissolved in (COCl)₂ (40 mL). The mixture was then stirred at 70° C. for 2 hrs. On completion, the reaction mixture was concentrated under reduced pressure to give the title compound (15.5 g, 99% yield) as white solid.

### Step 2—Methyl 3-bromobicyclo[1.1.1]pentane-1-carboxylate

A mixture of sodium; 1-oxidopyridin-1-ium-2-thiolate (13.4 g, 90.4 mmol, CAS #3811-73-2), methyl 3-chlorocarbonylbicyclo[1.1.1]pentane-1-carboxylate (15.5 g, 82.1 mmol), DMAP (502 mg, 4.11 mmol) and bromo(trichloro)methane (81.4 g, 410 mmol) in toluene (150 mL) was degassed and purged with N₂ three times. Then the mixture was stirred at 110° C. for 2 hrs under N₂ atmosphere and 200 W mercury lamp. On completion, the mixture was filtered, the organic phase was diluted with H₂O (300 mL) and extracted with EA (200 mL×3). The combined organic layers were, dried over Na₂SO₄, filtered and concentrated under reduced pressure to give a residue. The residue was purified by column chromatography (SiO₂, Petroleum ether/Ethyl acetate=50/1 to 20/1) to give the title compound (6.80 g, 40% yield) as colorless oil.

### Step 3—(3-Bromo-1-bicyclo[1.1.1]pentanyl)methanol

To a solution of methyl 3-bromobicyclo[1.1.1]pentane-1-carboxylate (5.80 g, 28.2 mmol) in THF (40 mL) was added LiAlH₄ (1.07 g, 28.2 mmol). The mixture was then stirred at 0° C. for 1 hr. On completion, the reaction mixture was quenched with H₂O (6 mL) at 0° C., and then 10% NaOH (10 mL) was added at 0° C. Then the organic layer was dried over Na₂SO₄, filtered and concentrated under reduced pressure to give the title compound (4.00 g, 79% yield) as yellow oil.

### Step 4—(3-Bromo-1-bicyclo[1.1.1]pentanyl)methoxy-trimethyl-silane

To a solution of (3-bromo-1-bicyclo[1.1.1]pentanyl)methanol (2.30 g, 12.9 mmol) in THF (20 mL) was added TEA (3.16 g, 31.1 mmol, 4.34 mL), then TMSCl (2.82 g, 25.9 mmol, 3.30 mL) was added at 0° C. dropwise. The reaction mixture was then stirred at 25° C. for 12 hrs. On completion, the reaction mixture was diluted with H₂O (30 mL) and extracted with EA 60 mL (2×30 mL). The combined organic layers were dried over Na₂SO₄, filtered and the filtrate was concentrated under reduced pressure to give the title compound (2.6 g, 80% yield) as yellow oil. ¹H NMR (400 MHz, CDCl₃) δ 3.64 (s, 2H), 2.16 (s, 6H), 0.10 (s, 9H).

### Step 5—Benzyl 4-[(3-bromo-1-bicyclo[1.1.1]pentanyl)methoxy]piperidine-1-carboxylate

To a solution of (3-bromo-1-bicyclo[1.1.1]pentanyl)methoxy-trimethyl-silane (2.60 g, 10.4 mmol) and benzyl 4-oxopiperidine-1-carboxylate (2.43 g, 10.4 mmol, 2.08 mL, CAS #19099-93-5) in DCM (20 mL) was added Et₃SiH (1.33 g, 11.4 mmol, 1.83 mL) and TMSOTf (1.16 g, 5.22 mmol, 942 uL) at −60° C. Then the reaction mixture was stirred at 0° C. for 1 hr. On completion, the reaction mixture was diluted with H₂O (30 mL) and extracted with DCM (2×30 mL). The combined organic layers were dried over Na₂SO₄, filtered and the filtrate was concentrated under reduced pressure to give a residue. The residue was purified by reverse phase (0.1% FA condition) to give the title compound (1.75 g, 42% yield) as a white solid. ¹H NMR (400 MHz, CDCl₃) δ 7.40-7.29 (m, 5H), 5.13 (s, 2H), 3.80-3.72 (m, 2H), 3.51 (s, 2H), 3.48-3.40 (m, 1H), 3.29-3.19 (m, 2H), 2.19 (s, 6H), 1.83-1.72 (m, 2H), 1.60-1.46 (m, 2H); LC-MS (ESI+) m/z 396.0 (M+H)⁺.

### 3-[1-(2,6-Dioxo-3-piperidyl)-3-methyl-2-oxo-benzimidazol-4-yl]bicyclo[1.1.1]pentane-1-carbaldehyde (Intermediate CGL)

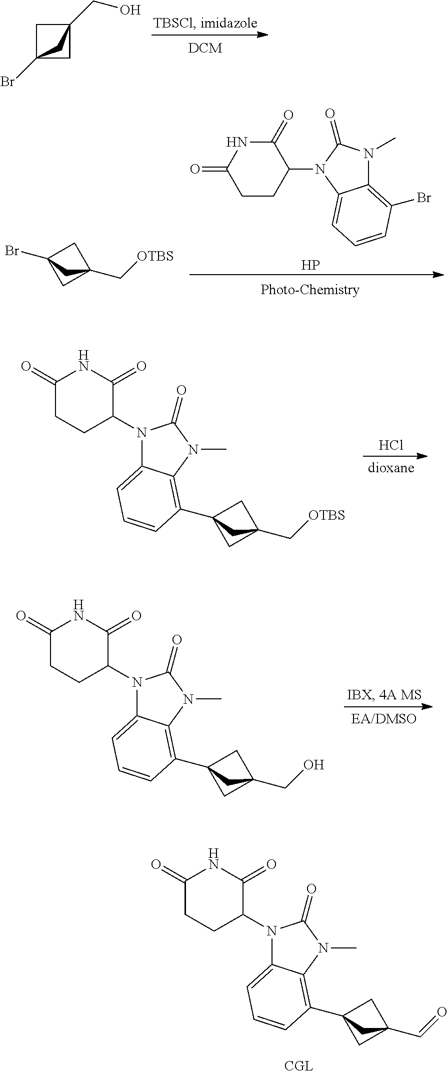

### Step 1—(3-Bromo-1-bicyclo[1.1.1]pentanyl)methoxy-tert-butyl-dimethyl-silane

To a solution of (3-bromo-1-bicyclo[1.1.1]pentanyl)methanol (2.50 g, 14.1 mmol, synthesized via Steps 1-2 of Intermediate CGJ) in DCM (30 mL) was added tert-butyl-chloro-dimethyl-silane (3.19 g, 21.1 mmol) and imidazole (1.92 g, 28.2 mmol). The mixture was stirred at 25° C. for 3 hrs. On completion, the reaction mixture was quenched with NH₄Cl saturated solution (60 mL) at 25° C., and then extracted with DCM 150 mL (50 mL×3). The combined organic layers were dried over Na₂SO₄, filtered and concentrated under reduced pressure to give a residue. The residue was purified by column chromatography (SiO₂, Petroleum ether) to give the title compound (1.60 g, 38% yield) as colorless oil. ¹H NMR (400 MHz, CDCl₃) δ 3.66 (s, 2H), 2.15 (s, 6H), 0.90-0.88 (m, 9H), 0.05-0.02 (m, 6H).

### Step 2—3-[4-[3-[[tert-butyl(dimethyl)silyl]oxymethyl]-1-bicyclo[1.1.1]pentanyl]-3-methyl-2-oxo-benzimidazol-1-yl]piperidine-2,6-dione

To a 40 mL vial equipped with a stir bar was added (3-bromo-1-bicyclo[1.1.1]pentanyl) methoxy-tert-butyl-dimethyl-silane (1.23 g, 4.23 mmol), 3-(4-bromo-3-methyl-2-oxo-benzimidazol-1-yl)piperidine-2,6-dione (1.10 g, 3.25 mmol, Intermediate HP), Ir[dF(CF₃)ppy]₂(dtbpy)(PF₆) (36.4 mg, 32.5 umol), NiCl₂.dtbbpy (19.4 mg, 48.7 umol), TTMSS (808 mg, 3.25 mmol), and 2,6-lutine (697 mg, 6.51 mmol) in DME (35 mL). The vial was sealed and placed under nitrogen was added. The reaction was stirred and irradiated with a 10 W blue LED lamp (3 cm away), with cooling water to keep the reaction temperature at 25° C. for 14 hrs. On completion, the mixture was filtered and concentrated under reduced pressure to give a residue. The residue was purified by prep-HPLC purification (column: Phenomenex Luna C18 200*40 mm*10 um; mobile phase: [water(FA)-ACN]; B %: 63%-93%, 10 min) to give the title compound (150 mg, 9% yield) as light yellow solid. LC-MS (ESI⁺) m/z 470.1 (M+H)⁺.

### Step 3—3-[4-[3-(Hydroxymethyl)-1-bicyclo[1.1.1]pentanyl]-3-methyl-2-oxo-benzimidazol-1-yl]piperidine-2,6-dione

3-[4-[3-[[tert-butyl(dimethyl)silyl]oxymethyl]-1-bicyclo[1.1.1]pentanyl]-3-methyl-2-oxo-benzimidazol-1-yl]piperidine-2,6-dione (95.0 mg, 202 umol) was dissolved in HCl/dioxane (3 mL). The mixture was then stirred at 25° C. for 1 hr. On completion, the reaction mixture was concentrated under reduced pressure to give the title compound (70.0 mg, 97% yield) as brown solid. LC-MS (ESI⁺) m/z 355.9 (M+H)⁺.

### Step 4—3-[1-(2,6-Dioxo-3-piperidyl)-3-methyl-2-oxo-benzimidazol-4-yl]bicyclo[1.1.1]pentane-1-carbaldehyde

To a solution of 3-[4-[3-(hydroxymethyl)-1-bicyclo[1.1.1]pentanyl]-3-methyl-2-oxo-benzimidazol-1-yl]piperidine-2,6-dione (70.0 mg, 196 umol) in EA (2 mL) and DMSO (0.3 mL) was added IBX (359 mg, 590 umol, 46% solution) and 4 Å molecular sieves (70.0 mg, 196 umol). The mixture was then stirred at 50° C. for 3 hrs. On completion, the reaction mixture was quenched with Na₂S₂O₃ (20 mL) and NaHCO₃ (20 mL) at 25° C., and then extracted with EA (20 mL×3). The combined organic layers were concentrated under reduced pressure to give a residue to give the title compound (60.0 mg, 86% yield) as light yellow solid. LC-MS (ESI⁺) m/z 372.1 (M+H₂O)+.

### 3-[3-Methyl-2-oxo-4-[3-(piperazin-1-ylmethyl)-1-bicyclo[1.1.1]pentanyl]benzimidazol-1-yl]piperidine-2,6-dione (Intermediate CGM)

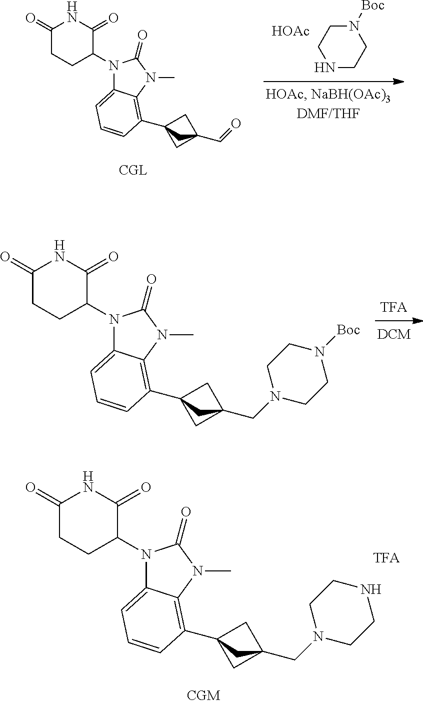 [SMILES: Cn1c(=O)n(C2CCC(=O)NC2=O)c2cccc([C@]34C[C@](C=O)(C3)C4)c21 ; CC(C)(C)OC(=O)N1CCNCC1 ; Cn1c(=O)n(C2CCC(=O)NC2=O)c2cccc([C@]34C[C@](CN5CCN(C(=O)OC(C)(C)C)CC5)(C3)C4)c21 ; "HOAc" ; Cn1c(=O)n(C2CCC(=O)NC2=O)c2cccc([C@]34C[C@](CN5CCNCC5)(C3)C4)c21]

### Step 1—Tert-butyl 4-[[3-[1-(2,6-dioxo-3-piperidyl)-3-methyl-2-oxo-benzimidazol-4-yl]-1-bicyclo[1.1.1]pentanyl]methyl]piperazine-1-carboxylate

To a solution of tert-butyl piperazine-1-carboxylate (209 mg, 848 umol, HOAc salt, CAS #143238-38-4) in DMF (1.5 mL) was added TEA (34.3 mg, 339 umol) at 25° C. until the pH stabilized at 8. The mixture was stirred at 25° C. for 0.5 hr, then AcOH (20.3 mg, 339 umol) was added at 25° C. to solution until pH stabilized at 5-6. The mixture was then cooled to −15° C. and 3-[1-(2,6-dioxo-3-piperidyl)-3-methyl-2-oxo-benzimidazol-4-yl]bicyclo[1.1.1]pentane-1-carbaldehyde (60.0 mg, 169 umol, Intermediate CGL) was added to the mixture which was then stirred for 0.5 hr. Next, NaBH(OAc)₃ (71.9 mg, 339 umol) was added one portion and the resulting reaction mixture was stirred at −15° C. for 1 hr. On completion, the reaction was quenched with H₂O (0.1 mL) at −15° C., and then filtered and concentrated under reduced pressure to give a residue. The residue was purified by prep-HPLC purification (column: Phenomenex Luna C18 150*25 mm*10 um; mobile phase: [water(HCl)-ACN]; B %: 13%-43%, 10 min) to give the title compound (10.0 mg, 11% yield) as yellow solid. LC-MS (ESI⁺) m/z 524.4 (M+H)⁺.

### Step 2—3-[3-Methyl-2-oxo-4-[3-(piperazin-1-ylmethyl)-1-bicyclo[1.1.1]pentanyl]benzimidazol-1-yl]piperidine-2,6-dione

To a solution of tert-butyl 4-[[3-[1-(2,6-dioxo-3-piperidyl)-3-methyl-2-oxo-benzimidazol-4-yl]-1-bicyclo [1.1.1]pentanyl]methyl]piperazine-1-carboxylate (10.0 mg, 19.1 umol) in DCM (1 mL) was added TFA (10.8 mg, 95.4 umol). The mixture was stirred at 25° C. for 1 hr. On completion, the mixture was concentrated under reduced pressure to give the title compound (10.0 mg, 97% yield, TFA) as yellow solid. LC-MS (ESI⁺) m/z 424.4 (M+H)⁺.

### 3-(4-bromo-3-cyclopropyl-2-oxo-benzimidazol-1-yl)piperidine-2,6-dione (Intermediate CGN)

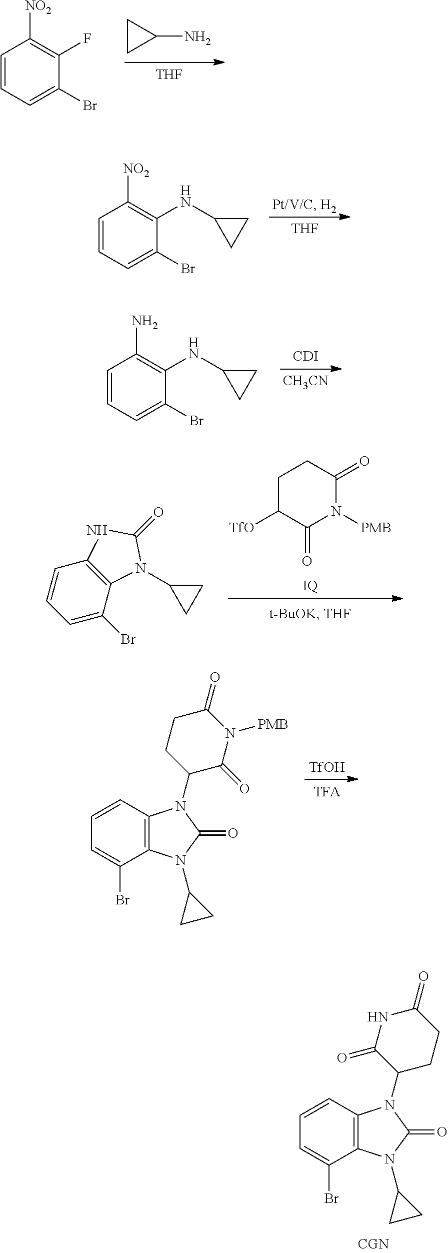

### Step 1—2-Bromo-N-cyclopropyl-6-nitro-aniline

To a solution of 1-bromo-2-fluoro-3-nitro-benzene (5 g, 22.7 mmol, CAS #58534-94-4) in THF (50 mL) was added cyclopropanamine (3.89 g, 68.1 mmol) at 0° C. under N₂. The reaction was then stirred at 25° C. for 16 hrs. On completion, the mixture was quenched with H₂O (100 mL), diluted with EA (100 mL), and extracted with EA (3×100 mL). The combined organic layer was washed with brine (3×100 mL), filtered and concentrated in vacuo to give the title compound (5.8 g, 99% yield) as orange oil. ¹H NMR (400 MHz, DMSO-d₆) δ 7.77-7.70 (m, 2H), 6.72 (t, J=8.0 Hz, 1H), 6.42 (s, 1H), 2.61-2.54 (m, 1H), 0.65-0.56 (m, 2H), 0.41-0.35 (m, 2H).

### Step 2—3-Bromo-N2-cyclopropyl-benzene-1,2-diamine

To a solution of 2-bromo-N-cyclopropyl-6-nitro-aniline (6.4 g, 24.8 mmol) in THF (90 mL) was added Pt/V/C (4.5 g, 17.2 mmol) under N₂. The suspension was degassed in vacuo and purged with H₂ three times. The mixture was then stirred under H₂ (15 psi) at 2 5° C. for 16 hours. On completion, the mixture was filtered and concentrated in vacuo to give the title compound (5.5 g, 97% yield) as brown oil. ¹H NMR (400 MHz, DMSO-d₆) δ 6.74-6.70 (m, 1H), 6.68-6.60 (m, 2H), 5.60-5.00 (m, 2H), 3.52-3.19 (m, 1H), 2.72-2.62 (m, 1H), 0.53-0.49 (m, 2H), 0.48-0.43 (m, 2H). LC-MS (ESI⁺) m/z 227.1 (M+H)⁺.

### Step 3—4-Bromo-3-cyclopropyl-1H-benzimidazol-2-one

To a solution of 3-bromo-N2-cyclopropyl-benzene-1,2-diamine (5.5 g, 24.2 mmol) in ACN (130 mL) was added CDI (7.85 g, 48.4 mmol). The mixture was then stirred at 80° C. for 3 hrs. On completion, the mixture was concentrated in vacuo. The crude product was triturated with H₂O (50 mL) for 30 min, filtered and the cake was dried in vacuo to give the title compound (5.4 g, 88% yield) as yellow solid. ¹H NMR (400 MHz, DMSO-d₆) δ 11.00 (s, 1H), 7.16 (dd, J=1.2, 8.0 Hz, 1H), 6.94-6.88 (m, 2H), 3.06-2.97 (m, 1H), 1.08-1.04 (m, 4H).

### Step 4—3-(4-Bromo-3-cyclopropyl-2-oxo-benzimidazol-1-yl)-1-[(4-methoxyphenyl)methyl]piperidine-2,6-dione

To a solution of 4-bromo-3-cyclopropyl-1H-benzimidazol-2-one (800 mg, 3.16 mmol) in THF (10 mL) was stirred at −10° C., then the t-BuOK (638 mg, 5.69 mmol) was added in above solution was stirred at −10° C. for 1.5 hrs. Then the solution of [1-[(4-methoxyphenyl)methyl]-2,6-dioxo-3-piperidyl]trifluoromethanesulfonate (1.45 g, 3.79 mmol, Intermediate IQ) in THF (15 mL) was dropwise into the above solution. The mixture was stirred at −10° C. for 2.5 hrs. On completion, the mixture was quenched with NH₄Cl (10 mL), diluted with H₂O (30 mL), and extracted with EA (2×50 mL). The combined organic layer was then washed with brine (2×50 mL), dried with anhydrous Na₂SO₄, filtered and concentrated in vacuo. The mixture was purified by reversed phase (0.1% FA) to give the title compound (1 g, 65% yield) as green solid. ¹H NMR (400 MHz, DMSO-d₆) δ 7.25 (d, J=8.4 Hz, 1H), 7.20 (d, J=8.4 Hz, 2H), 7.01 (s, 1H), 6.96-6.90 (m, 1H), 6.88-6.83 (m, 2H), 5.51 (dd, J=5.6, 13.0 Hz, 1H), 4.78 (q, J=14.4 Hz, 2H), 3.72 (s, 3H), 3.14-3.08 (m, 1H), 3.06-2.97 (m, 1H), 2.86-2.77 (m, 1H), 2.76-2.66 (m, 1H), 2.10-1.99 (m, 1H), 1.15-1.06 (m, 4H). LC-MS (ESI⁺) m/z 484.1 (M+H)⁺.

### Step 5—3-(4-Bromo-3-cyclopropyl-2-oxo-benzimidazol-1-yl)piperidine-2,6-dione

To a solution of 3-(4-bromo-3-cyclopropyl-2-oxo-benzimidazol-1-yl)-1-[(4-methoxyphenyl)methyl]piperidine-2,6-dione (400 mg, 825 umol) in TFA (5 mL) was added TfOH (100 uL). The mixture was then stirred at 70° C. for 5.5 hrs. On completion, the mixture was diluted with ACN (5 mL) and adjusted to pH=6-7 with TEA, then the mixture was concentrated in vacuo. The crude product was purified by reversed phase (0.1% FA) to give the title compound (106 mg, 35% yield) as white solid. ¹H NMR (400 MHz, DMSO-d₆) δ 11.10 (s, 1H), 7.26 (dd, J=1.2, 8.4 Hz, 1H), 7.12 (d, J=7.6 Hz, 1H), 7.01-6.95 (m, 1H), 5.35 (dd, J=5.6, 12.8 Hz, 1H), 3.14-3.07 (m, 1H), 2.92-2.81 (m, 1H), 2.74-2.60 (m, 2H), 2.05-1.96 (m, 1H), 1.15-1.05 (m, 4H). LC-MS (ESI⁺) m/z 366.0 (M+H)⁺.

### Tert-butyl 4-(3-bromocyclobutoxy)piperidine-1-carboxylate (Intermediate CGS)

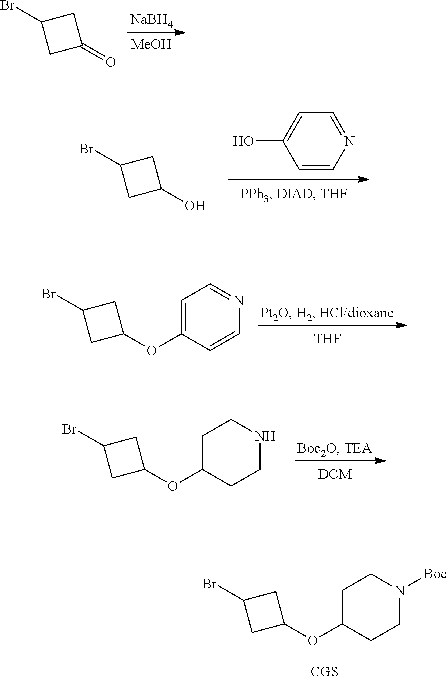

### Step 1—3-bromocyclobutanol

To a solution of 3-bromocyclobutanone (3.00 g, 20.1 mmol, CAS #23761-24-2) in MeOH (10 mL) was added NaBH₄ (1.95 g, 51.5 mmol) at 0° C. The reaction was then stirred at 25° C. for 2 hrs. On completion, the reaction was quenched with NH₄Cl saturated solution (7 mL). The mixture was diluted with EA (200 mL). The organic layer was washed with water (200 mL×3) and concentrated in vacuo to give title compound (3 g, 98% yield) as brown oil. ¹H NMR (400 MHz, CHCl₃) δ 4.11-4.02 (m, J=7.2 Hz, 1H), 3.92-3.83 (m, 1H), 3.08 (d, J=8.4 Hz, 1H), 3.04-2.94 (m, 2H), 2.44-2.34 (m, 2H).

### Step 2—4-(3-bromocyclobutoxy)pyridine

To a solution of 3-bromocyclobutanol (2.9 g, 19.2 mmol) and pyridin-4-ol (1.83 g, 19.1 mmol) in THF (10 mL) was added PPh₃ (7.56 g, 28.8 mmol) at 0° C. Then, DIAD (5.83 g, 28.8 mmol) was added into the above mixture at 0° C. Then the mixture was stirred at 50° C. for 16 hrs. On completion, the reaction was filtered and the filtrate was concentrated in vacuo. The residue was purified by column chromatography (SiO₂, Petroleum ether/Ethyl acetate=2/1 to 1/1) to give title compound (2 g, 45% yield) as colorless oil. ¹H NMR (400 MHz, CHCl₃) δ 8.47 (d, J=6.0 Hz, 2H), 6.80 (d, J=6.4 Hz, 2H), 5.22-5.12 (m, 1H), 5.05-4.90 (m, J=6.3, 12.5 Hz, 1H), 2.97-2.94 (m, 2H), 2.91-2.88 (m, 2H).

### Step 3—4-(3-bromocyclobutoxy)piperidine

To a solution of 4-(3-bromocyclobutoxy)pyridine (500 mg, 2.19 mmol) and HCl/dioxane (4 M, 500 uL) in THF (120 mL) was added PtO₂ (300 mg, 1.32 mmol) under N₂. The suspension was degassed under vacuo and purged with H₂ several times. The mixture was stirred under H₂ (50 psi) at 25° C. for 16 hours. On completion, the reaction was filtered and the filtrate was concentrated in vacuo to give title compound (350 mg, 68% yield) as colorless oil. ¹H NMR (400 MHz, DMSO-d₆) δ 5.30-5.13 (m, 1H), 4.87-4.69 (m, 2H), 3.19-3.09 (m, 1H), 3.05-2.98 (m, 1H), 2.97-2.82 (m, 6H), 2.69-2.56 (m, 3H), 2.00-1.84 (m, 1H), 1.65-1.53 (m, 1H).

### Step 4—Tert-butyl 4-(3-bromocyclobutoxy)piperidine-1-carboxylate

A mixture of 4-(3-bromocyclobutoxy)piperidine (300 mg, 1.28 mmol), TEA (259 mg, 2.56 mmol) and Boc₂O (559 mg, 2.56 mmol) in DCM (2 mL) was stirred at 25° C. for 2 hrs. On completion, the reaction was diluted with DCM (50 mL). The organic layer was washed with water (50 mL×3) and concentrated in vacuo. The residue was purified by column chromatography (SiO₂, Petroleum ether/Ethyl acetate=20/1 to 10/1, P1:R_{f}=0.2) to give title compound (250 mg, 58% yield) as yellow oil. LC-MS (ESI⁺) m/z 278.1 (M−56+H)⁺.

### 1-[8-[3-(4-Piperidyloxy)cyclobutyl]-4-isoquinolyl]hexahydropyrimidine-2,4-dione (Intermediate CGT)

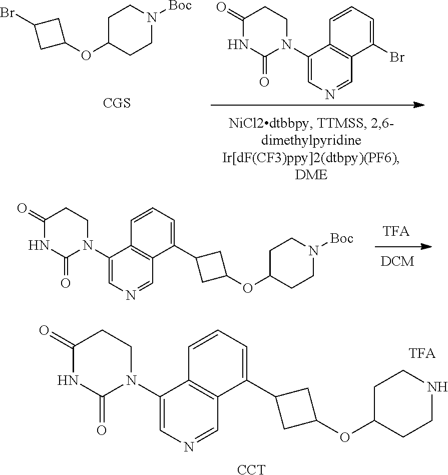

### Step 1—Tert-butyl 4-[3-[4-(2,4-dioxohexahydropyrimidin-1-yl)-8-isoquinolyl]cyclobutoxy]piperidine-1-carboxylate

To a solution of 1-(8-bromo-4-isoquinolyl)hexahydropyrimidine-2,4-dione (368 mg, 1.15 mmol, Intermediate CCT) and tert-butyl 4-(3-bromocyclobutoxy)piperidine-1-carboxylate (500 mg, 1.50 mmol, Intermediate CGS) in DME (3 mL) were added Ir[dF(CF3)ppy]2(dtbpy)(PF6) (25.8 mg, 23.0 umol,), NiCl2.dtbbpy (9.16 mg, 23.0 umol), TTMSS (286 mg, 1.15 mmol) and 2,6-dimethylpyridine (246 mg, 2.30 mmol). The vial was sealed and placed under nitrogen, then the reaction was stirred and irradiated with a 10 W blue LED lamp (3 cm away), with cooling water to keep the reaction temperature at 25° C. for 1 hr. On completion, the reaction mixture was filtered and concentrated in vacuo to give a residue. The residue was purified by reverse phase (FA condition) to give the title compound (200 mg, 35% yield) as white solid. ¹H NMR (400 MHz, DMSO-d₆) δ 10.6 (s, 1H), 9.45-9.30 (m, 1H), 8.58 (s, 1H), 7.90-7.84 (m, 1H), 7.82-7.76 (m, 1H), 7.75-7.55 (m, 1H), 4.35-4.18 (m, 1H), 3.98-3.88 (m, 1H), 3.86-3.75 (m, 1H), 3.74-3.62 (m, 3H), 3.58-3.52 (m, 1H), 3.51-3.48 (m, 1H), 3.05-2.87 (m, 4H), 2.80-2.70 (m, 1H), 2.66 (s, 1H), 2.54 (s, 2H), 2.18-2.03 (m, 1H), 1.85-1.71 (m, 2H), 1.39 (s, 9H); LC-MS (ESI⁺) m/z 495.1 (M+H)⁺.

### Step 2—1-[8-[3-(4-Piperidyloxy)cyclobutyl]-4-isoquinolyl]hexahydropyrimidine-2,4-dione

To a solution of tert-butyl 4-[3-[4-(2,4-dioxohexahydropyrimidin-1-yl)-8-isoquinolyl]cyclobutoxy]piperidine-1-carboxylate (50 mg, 101 umol) in DCM (1 mL) was added TFA (308 mg, 2.70 mmol). The mixture was then stirred at 25° C. for 1 hr. On completion, the reaction mixture was concentrated in vacuo to give the title compound (50 mg, 97% yield, TFA) as a red oil. LC-MS (ESI⁺) m/z 395.0 (M+H)⁺.

### N-(2-((1r, 3r)-3-formylcyclobutyl)-5-methoxybenzo[d]thiazol-6-yl)-6-(trifluoromethyl)pico linamide (Intermediate CGY)

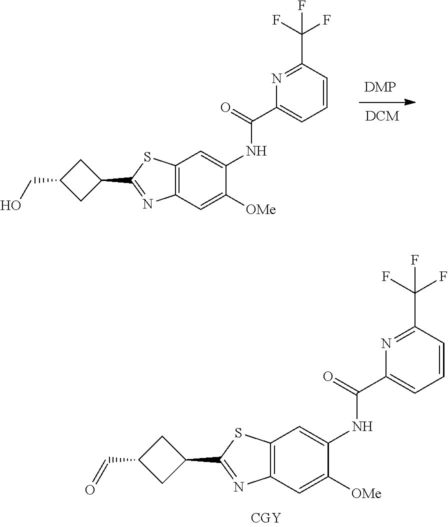 [SMILES: COc1cc2nc([C@H]3C[C@H](CO)C3)sc2cc1NC(=O)c1cccc(C(F)(F)F)n1 ; COc1cc2nc([C@H]3C[C@H](C=O)C3)sc2cc1NC(=O)c1cccc(C(F)(F)F)n1]

To a solution of N-[2-[3-(hydroxymethyl)cyclobutyl]-5-methoxy-1,3-benzothiazol-6-yl]-6-(trifluoromethyl)pyridine-2-carboxamide (280 mg, 640 umol, synthesized via Step 1 of Intermediate CAD) in DMF (2 mL) and THF (2 mL) was added DMP (407 mg, 960 umol). The mixture was then stirred at 25° C. for 16 hours. On completion, the reaction mixture was diluted with water (5 mL), then extracted with EA (3×5 ml). The combined organic layers were concentrated in vacuo to give the title compound (250 mg, 89% yield) as a yellow solid. ¹H NMR (400 MHz, CDCl₃) δ 10.64 (s, 1H), 9.87 (d, J=1.2 Hz, 1H), 9.11-8.94 (m, 1H), 8.43 (d, J=8.0 Hz, 1H), 8.07 (t, J=8.0 Hz, 1H), 7.95 (s, 1H), 7.81 (d, J=8.0 Hz, 1H), 7.51-7.45 (m, 1H), 4.05-3.97 (m, 3H), 3.86-3.24 (m, 1H), 2.82 (s, 2H), 2.79-2.72 (m, 1H), 2.71-2.60 (m, 2H).

### 3-[3-methyl-2-oxo-4-[3-(4-piperidyloxy)cyclobutyl]benzimidazol-1-yl]piperidine-2,6-dione (Intermediate CGZ)

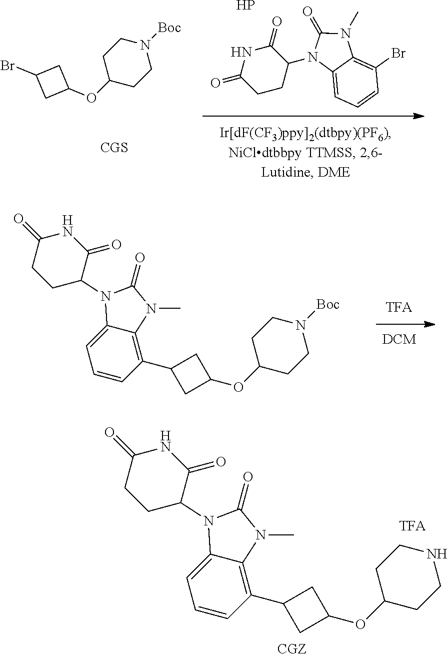

### Step 1—Tert-butyl 4-[3-[1-(2,6-dioxo-3-piperidyl)-3-methyl-2-oxo-benzimidazol-4-yl]cyclobutoxy]piperidine-1-carboxylate

To an 15 mL vial equipped with a stir bar was added 3-(4-bromo-3-methyl-2-oxo-benzimidazol-1-yl)piperidine-2,6-dione (67.6 mg, 0.2 mmol, Intermediate HP), tert-butyl 4-(3-bromocyclobutoxy)piperidine-1-carboxylate (86.9 mg, 260 umol, Intermediate CGS), Ir[dF(CF₃)ppy]₂(dtbpy)(PF₆) (2.24 mg, 2.00 umol), NiCl₂.dtbbpy (1.19 mg, 3.00 umol), TTMSS (49.7 mg, 200 umol) and 2,6-lutidine (42.8 mg, 400 umol) in DME (2 mL). The vial was sealed and placed under nitrogen then the reaction was stirred and irradiated with a 10 W blue LED lamp (3 cm away), with cooling water to keep the reaction temperature at 25° C. for 14 hrs. On completion, the reaction was diluted with EA (100 mL). The organic layer was washed with water (100 mL×3) and concentrated in vacuo. The residue was purified by prep-HPLC (column: Phenomenex luna C18 150*25 mm*10 um; mobile phase: [water (0.225% FA)-ACN]; B %: 36%-66%, 10 min) to give title compound (35.0 mg, 34% yield) as white solid. ¹H NMR (400 MHz, DMSO-d₆) δ 11.08 (s, 1H), 7.16-6.96 (m, 3H), 5.35 (dd, J=5.6, 12.4 Hz, 1H), 4.28-4.05 (m, 1H), 3.71-3.61 (m, 2H), 3.59-3.44 (m, 4H), 3.05-2.83 (m, 3H), 2.75-2.56 (m, 4H), 2.45-2.36 (m, 1H), 2.08-1.93 (m, 2H), 1.82-1.72 (m, 2H), 1.38 (s, 9H), 1.35-1.25 (m, 2H), 0.08-0.02 (m, 1H).

### Step 2—3-[3-methyl-2-oxo-4-[3-(4-piperidyloxy)cyclobutyl]benzimidazol-1-yl]piperidine-2,6-dione

A mixture of tert-butyl 4-[3-[1-(2,6-dioxo-3-piperidyl)-3-methyl-2-oxo-benzimidazol-4-yl]cyclobutoxy]piperidine-1-carboxylate (20.0 mg, 39.0 umol) and TFA (154 mg, 1.35 mmol) in DCM (0.5 mL) was stirred at 25° C. for 1 hr. On completion, the reaction was concentrated in vacuo to give title compound (20 mg, 97% yield, TFA) as yellow oil. LC-MS (ESI⁺) m/z 413.2 (M+H)⁺.

### Tert-butyl (4R)-4-[1-(2,6-dioxo-3-piperidyl)-3-methyl-2-oxo-benzimidazol-4-yl]-2,2-dimethyl-piperidine-1-carboxylate (Intermediate CHA)

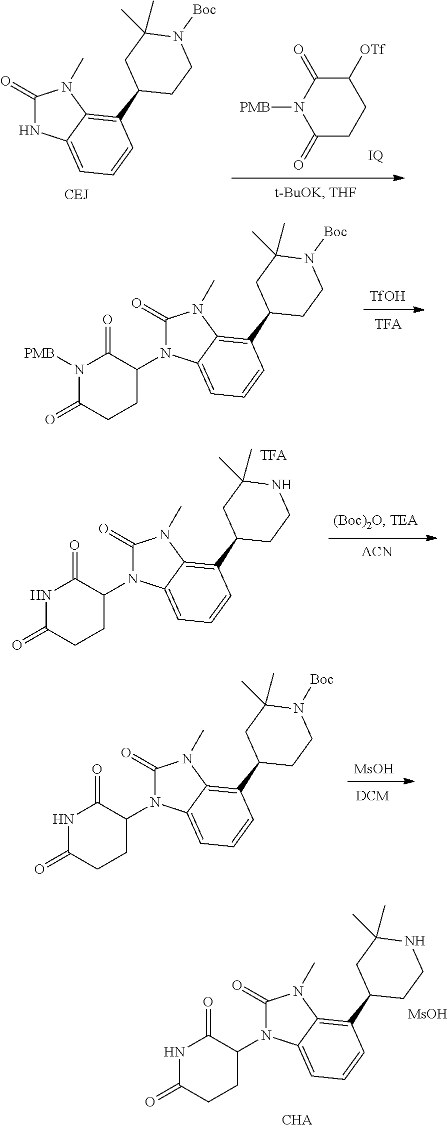

### Step 1—Tert-butyl (4R)-2,2-dimethyl-4-(3-methyl-2-oxo-1H-benzimidazol-4-yl)piperidine-1-carboxylate

To a solution of tert-butyl (4R)-2,2-dimethyl-4-[3-methyl-2-oxo-1-(2-trimethylsilylethoxymethyl) benzimidazol-4-yl]piperidine-1-carboxylate (240 mg, 490 umol, Intermediate CEJ) in THF (1 mL) was added TBAF (1 M, 2.45 mL), the reaction mixture was then stirred at 70° C. for 12 hrs. On completion, the reaction mixture was diluted with H₂O (50 mL), then extracted with EA (2×20 mL). The combined organic layers were washed with brine (30 mL), dried over Na₂SO₄, filtered and concentrated in vacuo to give the title compound (170 mg, 96.5% yield) as a white solid. ¹H NMR (400 MHz, DMSO-d₆) δ 10.86 (s, 1H), 6.97-6.89 (m, 2H), 6.83-6.81 (m, 1H), 3.89-3.86 (m, 1H), 3.62-3.48 (m, 4H), 3.21-3.15 (m, 1H), 1.93-1.82 (m, 1H), 1.77-1.63 (m, 3H), 1.49 (s, 3H), 1.41 (s, 9H), 1.36 (s, 3H). LC-MS (ESI⁺) m/z 303.9 (M−56+H)⁺

### Step 2—Tert-butyl (4R)-4-[1-[1-[(4-methoxyphenyl)methyl]-2,6-dioxo-3-piperidyl]-3-methyl-2-oxo-benzimidazol-4-yl]-2,2-dimethyl-piperidine-1-carboxylate

To a solution of tert-butyl (4R)-2,2-dimethyl-4-(3-methyl-2-oxo-1H-benzimidazol-4-yl)piperidine-1-carboxylate (170 mg, 472.93 umol) in THF (2 mL) was added tBuOK (79.6 mg, 709 umol) at −10° C. The mixture was stirred for 30 mins, then a solution of [1-[(4-methoxyphenyl) methyl]-2,6-dioxo-3-piperidyl]trifluoromethanesulfonate (234 mg, 614 umol, Intermediate IQ) in THF (2 mL) was added dropwise to the mixture, then the reaction mixture was stirred at −10° C. for 2 hrs. On completion, the reaction mixture was acidified with FA to pH=5˜6. Then the mixture was diluted with EA (100 mL), washed with NH₄Cl (100 mL) and brine (100 mL). The organic layer was dried over Na₂SO₄, filtered and concentrated in vacuo to give a residue. The residue was purified by reverse phase (0.10% FA condition) to give the title compound (200 mg, 71% yield) as a white solid. ¹H NMR (400 MHz, DMSO-d₆) δ 7.21 (d, J=8.4 Hz, 2H), 7.03-7.00 (m, 1H), 6.96-6.94 (m, 1H), 6.89-6.82 (m, 3H), 5.53 (dd, J=5.2, 12.8 Hz, 1H), 4.84-4.73 (m, 2H), 3.90-3.86 (m, 1H), 3.72 (s, 3H), 3.62 (s, 3H), 3.26-3.16 (m, 1H), 3.11-2.99 (m, 1H), 2.84-2.68 (m, 2H), 2.08-2.00 (m, 1H), 1.94-1.84 (m, 1H), 1.78-1.65 (m, 4H), 1.49 (s, 3H), 1.42 (s, 9H), 1.37 (s, 3H); LC-MS (ESI⁺) m/z 591.2 (M+H)⁺.

### Step 3—3-[4-[(4R)-2,2-dimethyl-4-piperidyl]-3-methyl-2-oxo-benzimidazol-1-yl]piperidine-2,6-dione

To a solution of tert-butyl (4R)-4-[1-[1-[(4-methoxyphenyl)methyl]-2,6-dioxo-3-piperidyl]-3-methyl-2-oxo-benzimidazol-4-yl]-2,2-dimethyl-piperidine-1-carboxylate (200 mg, 338 umol) in TFA (1.54 g, 13.5 mmol) was added TfOH (340 mg, 2.27 mmol), then the reaction was stirred at 70° C. for 2 hrs. On completion, the reaction mixture was concentrated in vacuo to give the title compound (160 mg, 97% yield) as yellow oil. LC-MS (ESI⁺) m/z 371.1 (M+H)⁺.

### Step 4—Tert-butyl (4R)-4-[1-(2,6-dioxo-3-piperidyl)-3-methyl-2-oxo-benzimidazol-4-yl]-2,2-dimethyl-piperidine-1-carboxylate

To a mixture of 3-[4-[(4R)-2,2-dimethyl-4-piperidyl]-3-methyl-2-oxo-benzimidazol-1-yl]piperidine-2,6-dione (160 mg, 330 umol, TFA) in TEA (100 mg, 990 umol, 137 uL) in ACN (3 mL) was added (Boc)₂O (79.2 mg, 363 umol, 83.4 uL), then the reaction was stirred at 25° C. for 16 hrs. On completion the reaction mixture was concentrated in vacuo to give a residue. The residue was purified by reverse phase (0.1% TFA condition) to give the title compound (120 mg, 77% yield) as a white solid. ¹H NMR (400 MHz, DMSO-d₆) δ 11.08 (s, 1H), 7.17-6.85 (m, 3H), 5.37 (dd, J=4.8, 12.0 Hz, 1H), 3.94-3.84 (m, 1H), 3.75-3.47 (m, 4H), 3.25-3.17 (m, 1H), 2.94-2.83 (m, 1H), 2.77-2.59 (m, 3H), 2.04-1.95 (m, 1H), 1.95-1.85 (m, 1H), 1.82-1.65 (m, 3H), 1.49 (s, 3H), 1.42 (s, 8H), 1.37 (s, 3H). LC-MS (ESI⁺) m/z 471.1 (M+H)⁺.

### Tert-butyl 4-((1s, 3s)-3-(1-(2,6-dioxopiperidin-3-yl)-3-methyl-2-oxo-2,3-dihydro-1H-benzo[d]imidazol-4-yl)cyclobutoxy)piperidine-1-carboxylate (Intermediate CIY) and tert-butyl 4-((1r, 3r)-3-(1-(2,6-dioxopiperidin-3-yl)-3-methyl-2-oxo-2,3-dihydro-1H-benzo[d]imidazol-4-yl)cyclobutoxy)piperidine-1-carboxylate (Intermediate CIZ)

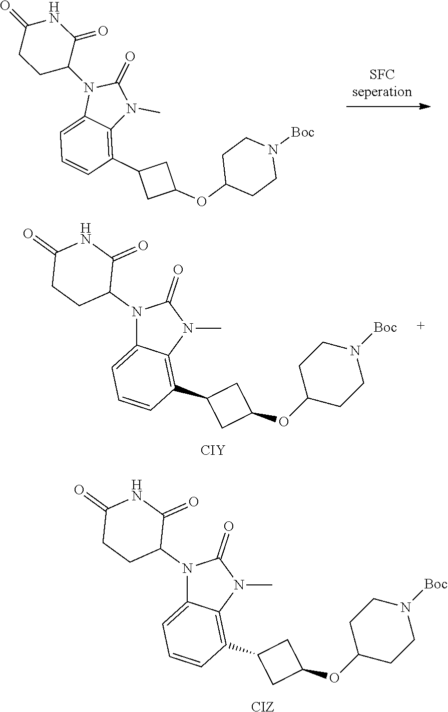

The tert-butyl 4-[3-[1-(2,6-dioxo-3-piperidyl)-3-methyl-2-oxo-benzimidazol-4-yl]cyclobutoxy]piperidine-1-carboxylate (50.0 mg, synthesized via Step 1 of Intermediate CGZ) was purified by SFC (column: DAICEL CHIRALPAK IC(250 mm*30 mm, 10 um); mobile phase: [Neu-MeOH]; B %: 70%-70%, 5.5; 40 min) (column: DAICEL CHIRALPAK AD(250 mm*30 mm, 10 um); mobile phase: [0.1% NH₃H₂O ETOH]; B %: 50%-50%, 3.4; 40 min) and concentrated in vacuo to give tert-butyl 4-((1s, 3s)-3-(1-(2,6-dioxopiperidin-3-yl)-3-methyl-2-oxo-2,3-dihydro-1H-benzo[d]imidazol-4-yl)cyclobutoxy)piperidine-1-carboxylate (26.0 mg, 96.5% yield) and tert-butyl 4-((1r, 3r)-3-(1-(2,6-dioxopiperidin-3-yl)-3-methyl-2-oxo-2,3-dihydro-1H-benzo[d]imidazol-4-yl)cyclobutoxy)piperidine-1-carboxylate (15.0 mg, 96.5% yield) as yellow solid. LC-MS (ESI⁺) m/z 535.3 (M+Na)+. Absolute stereochemistry isomers was assigned arbitrarily.

### 5-(trifluoromethyl)pyridine-3-carboxylic acid (CAS #131747-40-5) (Intermediate CBV)

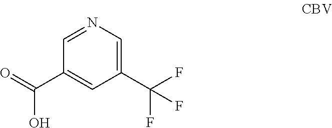 [SMILES: O=C(O)c1cncc(C(F)(F)F)c1]

### 3-[3-methyl-2-oxo-5-[(3S)-pyrrolidin-3-yl]benzimidazol-1-yl]piperidine-2,6-dione (Intermediate CJA)

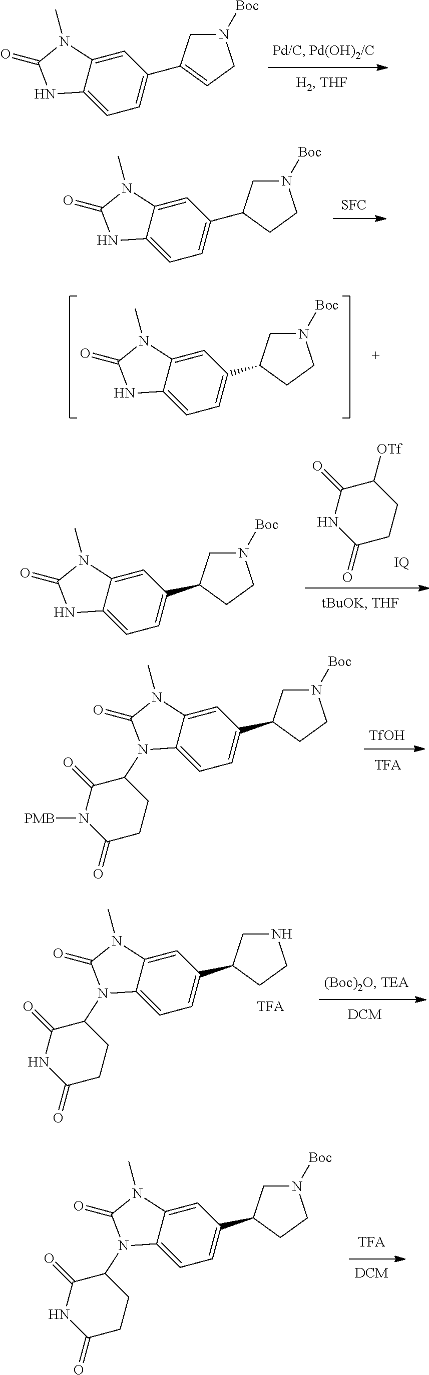

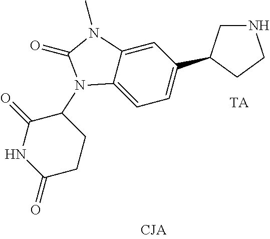

### Step 1—Tert-butyl 3-(3-methyl-2-oxo-1H-benzimidazol-5-yl)pyrrolidine-1-carboxylate

A mixture of tert-butyl 3-(3-methyl-2-oxo-1H-benzimidazol-5-yl)-2,5-dihydropyrrole-1-carboxylate (1 g, 3.17 mmol, synthesized via Step 1 of Intermediate CFH), Pd/C (500 mg, 10 wt %) and Pd(OH)₂/C (6.00 g, 4.27 mmol) in MeOH (5 mL) was degassed and purged with H₂ three times. Then the mixture was stirred at 25° C. for 2 hrs under H₂ atmosphere. On completion, the reaction mixture was filtered and concentrated in vacuo to give the title compound (1.0 g, 99% yield) as white solid. ¹H NMR (400 MHz, DMSO-d₆) δ 7.04 (s, 1H), 6.89 (s, 2H), 6.82-6.82 (m, 1H), 3.68 (s, 1H), 3.48 (s, 1H), 3.31 (d, J=4.0 Hz, 1H), 3.26 (s, 3H), 3.20-3.11 (m, 1H), 2.24-1.84 (m, 2H), 1.40 (s, 9H).

### Step 2—Tert-butyl (3S)-3-(3-methyl-2-oxo-1H-benzimidazol-5-yl)pyrrolidine-1-carboxylate and tert-butyl (3R)-3-(3-methyl-2-oxo-1H-benzimidazol-5-yl)pyrrolidine-1-carboxylate

Tert-butyl 3-(3-methyl-2-oxo-1H-benzimidazol-5-yl)pyrrolidine-1-carboxylate (2 g, 6.30 mmol) was purified by SFC (column: DAICEL CHIRALCEL OJ(250 mm*50 mm, 10 um); mobile phase: [0.1% NH3H2OMEOH]; B %: 30%-30%, 2; 160 min). After SFC purification, the eluent was concentrated or evaporated to remove organic solvents. The residual aqueous solution was lyophilized to tert-butyl (3S)-3-(3-methyl-2-oxo-1H-benzimidazol-5-yl)pyrrolidine-1-carboxylate (1 g, 3.15 mmol, 50% yield) (¹HNMR (400 MHz, DMSO-d₆) δ 10.73 (s, 1H), 7.05 (s, 1H), 6.89 (s, 2H), 3.68 (t, J=8.8 Hz, 1H), 3.56-3.35 (m, 2H), 3.26 (s, 3H), 3.20-3.12 (m, 1H), 2.16 (dd, J=4.8, 10.0 Hz, 1H), 2.09-1.84 (m, 2H), 1.41 (d, J=6.0 Hz, 9H)) and tert-butyl (3R)-3-(3-methyl-2-oxo-1H-benzimidazol-5-yl)pyrrolidine-1-carboxylate (800 mg, 40% yield).

### Step 3—Tert-butyl (3S)-3-[1-[1-[(4-methoxyphenyl)methyl]-2,6-dioxo-3-piperidyl]-3-methyl-2-oxo-benzimidazol-5-yl]pyrrolidine-1-carboxylate

To a solution of tert-butyl (3S)-3-(3-methyl-2-oxo-1H-benzimidazol-5-yl)pyrrolidine-1-carboxylate (450 mg, 1.42 mmol) in THF (6 mL) was added t-BuOK (238 mg, 2.13 mmol). Then, to the above mixture was added [1-[(4-methoxyphenyl)methyl]-2,6-dioxo-3-piperidyl]trifluoromethanesulfonate (810 mg, 2.13 mmol, Intermediate IQ), then the mixture was stirred at 0° C. for 2 hrs. On completion, the reaction mixture was concentrated in vacuo. The residue was diluted with water (100 mL) and extracted with EA (40 mL×2). The combined organic layer was dried over Na₂SO₄, filtered and concentrated in vacuo. The residue was purified by prep-HPLC (column: Phenomenex Luna C18 200*40 mm*10 um; mobile phase: [water (FA)-ACN]; B %: 47%-77%, 10 min) to give the title compound (600 mg, 77% yield) as a white solid. ¹H NMR (400 MHz, DMSO-d₆) δ 7.23-7.14 (m, 3H), 6.92 (s, 2H), 6.85 (d, J=8.4 Hz, 2H), 5.50 (dd, J=5.6, 12.8 Hz, 1H), 4.87-4.71 (m, 2H), 3.72 (s, 3H), 3.70-3.65 (m, 1H), 3.49 (t, J=8.4 Hz, 1H), 3.42-3.36 (m, 1H), 3.34 (s, 3H), 3.27 (d, J=1.6 Hz, 1H), 3.23-3.15 (m, 1H), 3.12-3.00 (m, 1H), 2.86-2.69 (m, 2H), 2.21-1.93 (m, 3H), 1.41 (d, J=6.0 Hz, 9H).

### Step 4—3-[3-methyl-2-oxo-5-[(3S)-pyrrolidin-3-yl]benzimidazol-1-yl]piperidine-2,6-dione

To a solution of tert-butyl (3S)-3-[1-[1-[(4-methoxyphenyl)methyl]-2,6-dioxo-3-piperidyl]-3-methyl-2-oxo-benzimidazol-5-yl]pyrrolidine-1-carboxylate (200 mg, 364 umol) in TFA (2 mL) was added TfOH (711 mg, 4.74 mmol). The mixture was then stirred at 25° C. for 3 hrs. On completion, the reaction mixture was concentrated under reduced pressure to give the title compound (118 mg, 99% yield) as black brown oil. LC-MS (ESI⁺) m/z 329.2 (M+H)⁺.

### Step 5—Tert-butyl (3S)-3-[1-(2,6-dioxo-3-piperidyl)-3-methyl-2-oxo-benzimidazol-5-yl]pyrrolidine-1-carboxylate

To a solution of 3-[3-methyl-2-oxo-5-[(3S)-pyrrolidin-3-yl]benzimidazol-1-yl]piperidine-2,6-dione (110 mg, 334 umol) in DCM (5 mL) was added TEA (101 mg, 1.00 mmol) at 0° C. Then, to the above mixture was added (Boc)₂O (73.1 mg, 334 umol). The mixture was stirred at 25° C. for 3 hrs. On completion, the residue was diluted with water (30 mL) and extracted with DCM (20 mL×2). The combined organic layer was dried over Na₂SO₄, filtered and concentrated in vacuo. The residue was purified by prep-HPLC(column: Phenomenex luna C18 150*25 mm*10 um; mobile phase: [water(FA)-ACN]; B %: 28%-58%, 10.5 min) to give the title compound (135 mg, 92% yield) as white solid. ¹H NMR (400 MHz, DMSO-d₆) δ 11.08 (s, 1H), 7.17 (s, 1H), 7.10-7.00 (m, 1H), 7.00-6.92 (m, 1H), 5.34 (dd, J=5.6, 12.8 Hz, 1H), 3.69 (t, J=8.4 Hz, 1H), 3.54-3.45 (m, 1H), 3.36 (s, 3H), 3.31-3.24 (m, 2H), 3.24-3.13 (m, 1H), 2.96-2.84 (m, 1H), 2.77-2.58 (m, 2H), 2.18 (dd, J=5.6, 11.2 Hz, 1H), 2.07-1.93 (m, 2H), 1.41 (d, J=6.4 Hz, 9H); LC-MS (ESI+) m/z 373.1 (M−56).

### Step 6—3-[3-methyl-2-oxo-5-[(3S)-pyrrolidin-3-yl]benzimidazol-1-yl]piperidine-2,6-dione

To a solution of tert-butyl (3S)-3-[1-(2,6-dioxo-3-piperidyl)-3-methyl-2-oxo-benzimidazol-5-yl]pyrrolidine-1-carboxylate (65 mg, 151 umol) in DCM (2 mL) was added TFA (17.3 mg, 151 umol). The mixture was then stirred at 25° C. for 1 hr. On completion, the reaction mixture was concentrated under reduced pressure to give the title compound (65.0 mg, 96% yield) as a brown oil. LC-MS (ESI+) m/z 329.3 (M+H)⁺.

### Example 1 (Method 1): Synthesis of N-[2-[4-[4-[1-(2,6-dioxo-3-piperidyl)-3-methyl-2-oxo-benzimidazol-4-yl]piperidine-1-carbonyl]cyclohexyl]indazol-5-yl]-6-(trifluoromethyl)pyridine-2-carboxamide (I-94)

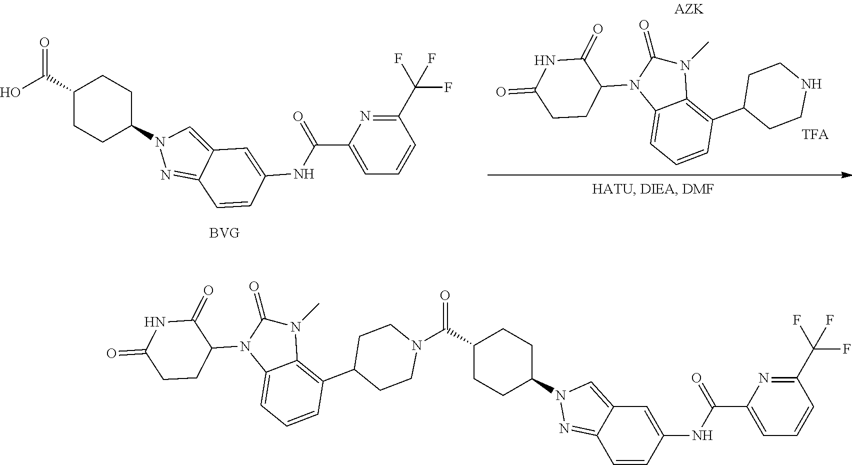 [SMILES: O=C(Nc1ccc2nn([C@H]3CC[C@H](C(=O)O)CC3)cc2c1)c1cccc(C(F)(F)F)n1 ; Cn1c(=O)n(C2CCC(=O)NC2=O)c2cccc(C3CCNCC3)c21 ; Cn1c(=O)n(C2CCC(=O)NC2=O)c2cccc(C3CCN(C(=O)[C@H]4CC[C@H](n5cc6cc(NC(=O)c7cccc(C(F)(F)F)n7)ccc6n5)CC4)CC3)c21]

To a mixture of 3-[3-methyl-2-oxo-4-(4-piperidyl)benzimidazol-1-yl]piperidine-2,6-dione (130 mg, 284 umol, TFA, Intermediate AZK) and DIEA (110 mg, 854 umol, 148 uL) in DMF (2 mL) was added in 4-[5-[[6-(trifluoromethyl)pyridine-2-carbonyl]amino]indazol-2-yl]cyclohexanecarboxylic acid (123 mg, 284 umol, Intermediate BVG) and HATU (129 mg, 341 umol). The reaction mixture was then stirred at 25° C. for 1.5 hours. On completion, the reaction mixture was quenched with water (0.05 mL). The residue was purified by prep-HPLC(column: Phenomenex luna C18 150*25 mm*10 um; mobile phase: [water (0.225% FA)-ACN]; B %: 42-72%, 10 min) to give the title compound (56.8 mg, 25% yield) as light yellow solid. ¹H NMR (400 MHz, DMSO-d₆) δ 11.10 (s, 1H), 10.36 (s, 1H), 8.42-8.29 (s, 4H), 8.17 (d, J=7.6 Hz, 1H), 7.64-7.53 (m, 2H), 7.03-6.99 (m, 2H), 5.38 (dd, J=5.2, 12.4 Hz, 1H), 4.62 (d, J=13.2 Hz, 1H), 4.57-4.47 (m, 1H), 4.18 (d, J=12.0 Hz, 1H), 3.64 (s, 3H), 3.55 (t, J=11.6 Hz, 1H), 3.29-3.19 (m, 1H), 2.95-2.80 (m, 2H), 2.77-2.63 (m, 31H), 2.19 (d, J=10.4 Hz, 2H), 2.13-1.81 (m, 8H), 1.77-1.62 (m, 3H), 1.61-1.51 (m, 1H); LC-MS (ESI⁺) m/z 757.6 (M+H)⁺.

The following compounds in Table 3 where prepared according to the same procedure of Method 2 above.

TABLE 3
Compounds synthesized via Method 1.

| | Inter- | Inter- | LCMS | |
|---|---|---|---|---|
| | mediate | mediate | (ES+) m/z | |
| I-#^{a} | Amine | Acid | (M + H)⁺ | ¹HNMR (400 MHz, DMSO-d6) δ |
| I-95^{b} | BUP | BUW | 740.6 | 11.09 (s, 1H), 10.22 (d, J = 2.0 Hz, 1H), 9.82 (s, 1H), 9.16 (d, |
| | | | | J = 2.0 Hz, 1H), 8.93 (s, 1H), 8.39 (s, 1H), 8.23 (s, 1H), 7.63 |
| | | | | (d, J = 8.4 Hz, 1H), 7.35 (d, J = 10.8 Hz, 1H), 7.05-6.95 (m, |
| | | | | 3H), 5.37 (dd, J = 6.0, 12.8 Hz, 1H), 4.48-4.40 (m, 1H), 3.59 |
| | | | | (s, 3H), 3.04-2.97 (m, 2H), 2.94-2.83 (m, 1H), 2.76-2.68 |
| | | | | (m, 1H), 2.65-2.58 (m, 1H), 2.26-2.21 (m, 2H), 2.19-2.15 |
| | | | | (m, 2H), 2.12-2.05 (m, 2H), 2.03-1.93 (m, 5H), 1.86-1.75 |
| | | | | (m, 5H), 1.71-1.64 (m, 1H), 1.17-1.11 (m, 2H) |
| I-186 | AQK | BVG | 786.7 | 11.09 (s, 1H), 10.36 (s, 1H), 8.42-8.39 (m, 2H), 8.38-8.34 |
| | | | | (m, 1H), 8.30 (s, 1H), 8.19-8.16 (m, 1H), 7.63-7.54 (m, 2H), |
| | | | | 7.01-6.94 (m, 2H), 6.91-6.87 (m, 1H), 5.39-5.33 (m, 1H), |
| | | | | 4.55-4.42 (m, 1H), 3.66 (s, 3H), 3.29 (s, 1H), 3.19-3.15 (m, |
| | | | | 2H), 2.99 (s, 3H), 2.90-2.82 (m, 2H), 2.79 (s, 2H), 2.76-2.70 |
| | | | | (m, 1H), 2.65-2.59 (m, 1H), 2.22-2.12 (m, 3H), 2.08-1.98 |
| | | | | (m, 3H), 1.92-1.83 (m, 3H), 1.74-1.62 (m, 3H), 1.60-1.55 |
| | | | | (m, 1H) |
| I-187 | AQK | BXH | 787.6 | 11.09 (s, 1H), 10.20 (s, 1H), 9.06 (s, 1H), 8.60 (s, 1H), 8.51- |
| | | | | 8.47 (m, 2H), 8.42 (t, J = 7.6 Hz, 1H), 8.26-8.22 (m, 1H), |
| | | | | 7.02-6.95 (m, 2H), 6.92-6.86 (m, 1H), 5.39-5.32 (m, 1H), |
| | | | | 4.71-4.62 (m, 1H), 4.52-4.37 (m, 1H), 3.67 (s, 3H), 3.20- |
| | | | | 3.14 (m, 2H), 3.00 (s, 3H), 2.91-2.75 (m, 5H), 2.64 (s, 1H), |
| | | | | 2.21-2.17 (m, 3H), 2.08-2.02 (m, 2H), 1.94-1.85 (m, 3H), |
| | | | | 1.75-1.63 (m, 3H), 1.62-1.53 (m, 2H) |
| I-188 | AZK | BXH | 758.6 | 11.17-11.00 (m, 1H), 10.20 (s, 1H), 9.06 (s, 1H), 8.60 (s, 1H), |
| | | | | 8.53-8.46 (m, 2H), 8.45-8.38 (m, 1H), 8.26-8.20 (m, 1H), |
| | | | | 7.00 (s, 3H), 5.41-5.35 (m, 1H), 4.73-4.54 (m, 2H), 4.25- |
| | | | | 4.11 (m, 1H), 3.64 (s, 3H), 3.59-3.51 (m, 1H), 2.95-2.82 (m, |
| | | | | 2H), 2.76-2.63 (m, 3H), 2.20 (s, 3H), 2.15-2.07 (m, 3H), |
| | | | | 1.97-1.88 (m, 4H), 1.74-1.64 (m, 3H), 1.58-1.51 (m, 1H) |
| I-309 | CEY | CEX | 747.3 | 11.10 (s, 1H), 8.36 (d, J = 8.0 Hz, 1H), 8.16 (d, J = 8.4 Hz, |
| | | | | 1H), 7.98 (s, 1H), 7.65 (d, J = 2.0 Hz, 1H), 7.52 (dd, J = 2.0, |
| | | | | 8.4 Hz, 1H), 7.23 (d, J = 8.4 Hz, 1H), 7.12-6.95 (m, 3H), 5.38 |
| | | | | (dd, J = 4.8, 12.4 Hz, 1H), 4.36-4.25 (m, 1H), 3.78-3.65 (m, |
| | | | | 1H), 3.61 (s, 3H), 3.56-3.42 (m, 2H), 2.95-2.85 (m, 2H), |
| | | | | 2.77-2.70 (m, 1H), 2.65-2.61 (m, 1H), 2.55-2.52 (m, 3H), |
| | | | | 2.05-1.81 (m, 10H), 1.77-1.65 (m, 1H), 1.42-1.30 (m, 2H), |
| | | | | 1.19 (s, 3H), 1.18 (s, 3H), 1.12-1.01 (m, 2H) |
| I-310 | CFA | CEZ | 729.3 | 10.55 (s, 1H), 9.66 (br s, 1H), 8.59 (s, 1H), 8.40 (br d, J = 7.6 |
| | | | | Hz, 1H), 8.21-8.11 (m, 1H), 7.98 (s, 1H), 7.90 (br d, J = 8.4 |
| | | | | Hz, 1H), 7.85-7.78 (m, 1H), 7.60 (br d, J = 1.2 Hz, 2H), 7.56 |
| | | | | (br d, J = 8.4 Hz, 1H), 7.22 (d, J = 8.4 Hz, 1H), 4.27 (br dd, |
| | | | | J = 6.4, 13.6 Hz, 1H), 4.02-3.98 (m, 1H), 3.87-3.49 (m, 4H), |
| | | | | 3.18-2.92 (m, 3H), 2.78 (td, J = 5.6, 16.8 Hz, 3H), 2.04 (br s, |
| | | | | 4H), 1.92 (br d, J = 10.4 Hz, 4H), 1.79-1.63 (m, 1H), 1.36 (br |
| | | | | d, J = 12.4 Hz, 2H), 1.20 (d, J = 6.4 Hz, 7H), 1.12-1.01 (m, 2H) |
| I-316 | CBP | CFF | 806.3 | 11.11-11.03 (m, 1H), 10.51 (d, J = 2.4 Hz, 1H), 9.00 (d, J = |
| | | | | 4.0 Hz, 1H), 8.51-8.45 (m, 1H), 8.44-8.39 (m, 1H), 8.24 (d, |
| | | | | J = 7.6 Hz, 1H), 7.69 (d, J = 9.2 Hz, 1H), 7.60 (q, J = 7.6 Hz, |
| | | | | 1H), 7.11-7.01 (m, 1H), 6.79-6.64 (m, 1H), 5.11-4.93 (m, |
| | | | | 1H), 4.04 (d, J = 6.0 Hz, 3H), 3.48-3.41 (m, 1H), 3.40-3.35 |
| | | | | (m, 2H), 3.28-3.22 (m, 1H), 3.16-2.97 (m, 2H), 2.96-2.78 |
| | | | | (m, 2H), 2.76-2.68 (m, 1H), 2.61 (d, J = 1.6 Hz, 1H), 2.47- |
| | | | | 2.35 (m, 2H), 2.19 (d, J = 11.2 Hz, 1H), 2.13-1.98 (m, 2H), |
| | | | | 1.93-1.80 (m, 2H), 1.78-1.63 (m, 4H), 1.62-1.50 (m, 2H), |
| | | | | 1.45-1.32 (m, 1H) |
| I-352 | CCC | AMY | 887.8 | 12.35 (d, J = 4.0 Hz, 1 H) 11.10 (d, J = 3.6 Hz, 1 H) 8.71 (d, |
| | | | | J = 5.2 Hz, 1 H) 8.42-8.47 (m, 1 H) 8.32-8.39 (m, 2 H) 8.16 |
| | | | | (d, J = 8.0 Hz, 1H) 7.50-7.63 (m, 2H)7.07 (dd, J = 12.0, 7.2 |
| | | | | Hz, 1 H) 6.94 (dd, J = 15.6, 8.4 Hz, 1 H) 6.47-6.56 (m, 1 H) |
| | | | | 5.93 (d, J = 6.0 Hz, 1 H) 5.05 (dt, J = 12.4, 4.8 Hz, 1 H) 4.49 |
| | | | | (d, J = 4.4 Hz, 1 H) 4.08-4.25 (m, 2 H) 3.45 (s, 1 H) 3.36 (s, |
| | | | | 1 H) 3.28 (s, 1 H) 2.81-3.07 (m, 4 H) 2.73 (s, 1 H) 2.55 (s, 3 |
| | | | | H) 2.36-2.41 (m, 2 H) 2.21-2.31 (m, 2 H) 2.13-2.20 (m, 2 |
| | | | | H) 1.97-2.06 (m, 3 H) 1.77-1.90 (m, 3 H) 1.69 (d, J = 6.8 |
| | | | | Hz, 2 H) 1.60 (d, J = 12.4 Hz, 8 H) |
| I-407 | CCJ | CEX | 748.3 | 11.10 (s, 1H), 8.37 (d, J = 7.2 Hz, 1H), 8.19-8.10 (m, 1H), |
| | | | | 7.98 (s, 1H), 7.65 (d, J = 2.0 Hz, 1H), 7.56-7.50 (m, 1H), 7.23 |
| | | | | (d, J = 8.4 Hz, 1H), 7.07-7.00 (m, 1H), 6.96 (d, J = 6.4 Hz, |
| | | | | 2H), 5.42-5.32 (m, 1H), 4.31 (d, J = 6.3, 8.0 Hz, 1H), 3.75- |
| | | | | 3.69 (m, 1H), 3.63 (s, 3H), 3.60-3.54 (m, 1H), 3.20-3.00 (m, |
| | | | | 5H), 2.96-2.84 (m, 2H), 2.76-2.70 (m, 1H), 2.69-2.63 (m, |
| | | | | 2H), 2.03-1.96 (m, 1H), 1.86 (d, J = 8.8 Hz, 4H), 1.78-1.65 |
| | | | | (m, 1H), 1.40-1.30 (m, 2H), 1.27-1.22 (m, 1H), 1.19 (d, J = |
| | | | | 6.4 Hz, 6H), 1.16-1.07 (m, 3H) |
| I-408 | CGB | CEZ | 730.4 | 0.52 (s, 1H), 9.44 (s, 1H), 8.54 (s, 1H), 8.35 (d, J = 7.6 Hz, |
| | | | | 1H), 8.19-8.11 (m, 1H), 7.98 (s, 1H), 7.77-7.70 (m, 1H), |
| | | | | 7.68-7.63 (m, 1H), 7.62 (d, J = 1.6 Hz, 1H), 7.55-7.49 (m, |
| | | | | 1H), 7.33-7.27 (m, 1H), 7.21 (d, J = 8.4 Hz, 1H), 4.32-4.21 |
| | | | | (m, 1H), 3.95-3.86 (m, 1H), 3.75-3.65 (m, 2H), 3.27-3.09 |
| | | | | (m, 6H), 3.03-2.90 (m, 2H), 2.81-2.70 (m, 2H), 1.88 (d, J = |
| | | | | 10.0 Hz, 4H), 1.71-1.50 (m, 1H), 1.41-1.29 (m, 2H), 1.27- |
| | | | | 1.23 (m, 2H), 1.19 (d, J = 6.4 Hz, 6H), 1.11-0.91 (m, 2H) |
| I-409 | CFA | CEX | 729.2 | 10.54 (s, 1H), 9.62 (s, 1H), 8.58 (s, 1H), 8.35 (d, J = 7.2 Hz, |
| | | | | 1H), 8.17 (d, J = 8.4 Hz, 1H), 7.98 (s, 1H), 7.88 (d, 1H), 7.80 |
| | | | | (t, 1H), 7.62 (t, 2H), 7.52 (d, J = 8.4 Hz, 1H), 7.23 (d, J = 8.4 |
| | | | | Hz, 1H), 4.36-4.26 (m, 1H), 3.96-3.88 (m, 1H), 3.74-3.68 |
| | | | | (m, 2H), 3.30 (s, 2H), 3.04-2.92 (m, 2H), 2.81-2.70 (m, 2H), |
| | | | | 2.03-1.82 (m, 9H), 1.67-1.54 (m, 1H), 1.39-1.21 (m, 4H), |
| | | | | 1.19 (d, J = 6.4 Hz, 6H), 1.08-0.95 (m, 2H) |
| I-410 | CCJ | CEZ | 748.2 | 0.90-1.02 (m, 2 H) 1.18 (d, J = 6.4 Hz.6 H) 1.25-1.37 (m, 2 H) |
| | | | | 1.43-1.56 (m, 1 H) 1.84 (d, J = 10.8 Hz, 4 H) 1.95-2.04 (m, |
| | | | | 1 H) 2.20 (s, 3 H) 2.61-2.75 (m, 3 H) 2.77-3.08 (m, 7 H) 3.62 |
| | | | | (s, 3 H) 3.65-3.74 (m, 1 H) 4.19-4.32 (m, 1 H) 5.35 (dd, J = 12.4, |
| | | | | 5.2 Hz, 1 H) 6.86-7.02 (m, 3 H) 7.21 (d, J = 8.4 Hz, 1 H) 7.52 |
| | | | | (dd, J = 8.4, 1.6 Hz, 1 H) 7.62 (d, J = 1.6 Hz, 1 H) 7.97 (s, 1 H) |
| | | | | 8.18 (d, J = 8.4 Hz, 1 H) 8.34 (d, J = 1.6 Hz, 1 H) 11.09 (s, 1 H) |

^{a}Coupling ran from 1-4 hrs from 0° C.-rt. ^{b}(COCl)₂ was used in place of HATU for the coupling, which was run at rt for 2.5 hrs with TEA in DCM.

### Example 2 (Method 2): Synthesis of N-[2-[4-[[4-[4-(2,4-dioxohexahydropyrimidin-1-yl)-8-isoquinolyl]-1-piperidyl]methyl]cyclohexyl]-6-(1-hydroxy-1-methyl-ethyl)indazol-5-yl]-6-(trifluoromethyl)pyridine-2-carboxamide (I-78)

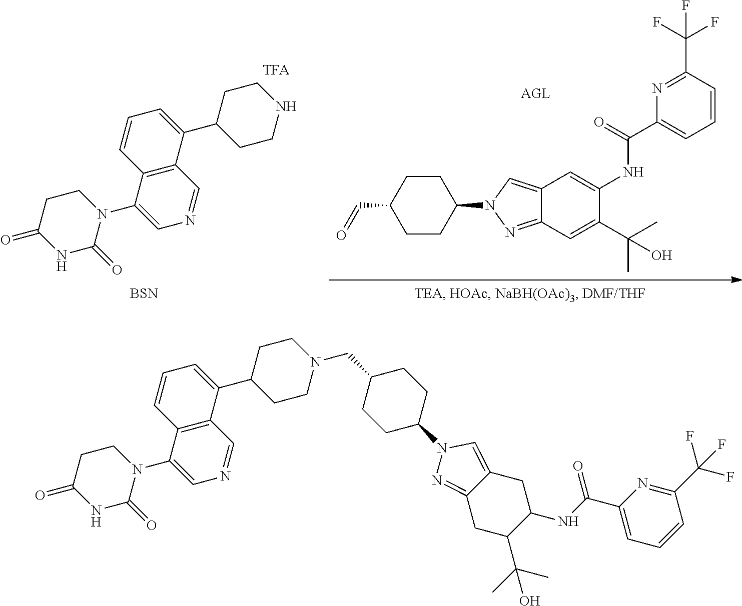

To a mixture of 1-[8-(4-piperidyl)-4-isoquinolyl]hexahydropyrimidine-2,4-dione (50.0 mg, 114 umol, TFA, Intermediate BSN) in DMF (0.5 mL) and THF (3 mL) was added TEA (11.5 mg, 114 umol, 15.8 uL) and the reaction mixture was stirred at −10° C. for 12 min. Then N-[2-(4-formylcyclohexyl)-6-(1-hydroxy-1-methyl-ethyl)indazol-5-yl]-6-(trifluoromethyl)pyridine-2-carboxamide (54.1 mg, 114 umol, Intermediate AGL) and HOAc (6.85 mg, 114 umol, 6.52 uL) was added to the mixture and the mixture was stirred at −10° C. for 0.5 hour. Next, NaBH(OAc)₃ (36.2 mg, 171 umol) was added to the mixture at −10° C. and the reaction mixture was stirred at −10° C. for 1 hour. On completion, the reaction mixture was quenched with water (0.05 mL) and concentrated in vacuo. The residue was purified by prep-HPLC (column: Welch Xtimate C18 150*25 mm*5 um; mobile phase: [water(0.225% FA)-ACN]; B %: 17%-47%11 min) to give the title compound (84.2 mg, 87 yield, FA) as white solid. ¹H NMR (400 MHz, DMSO-d₆) δ 12.37 (s, 1H), 10.56 (s, 1H), 9.68 (s, 1H), 8.73 (s, 1H), 8.61 (s, 1H)), 8.48-8.44 (m, 1H), 8.41-8.35 (m, 2H), 8.17 (dd, J=0.8, 7.6 Hz, 1H), 7.94-7.89 (m, 1H), 7.83 (t, J=7.8 Hz, 1H), 7.64 (d, J=6.8 Hz, 1H), 7.58 (s, 1H), 5.96 (s, 1H), 4.54-4.44 (m, 1H), 3.94 (ddd, J=5.2, 9.6, 12.0 Hz, 1H), 3.90-3.79 (m, 1H), 3.72 (td, J=6.0, 12.0 Hz, 1H), 3.57-3.44 (m, 2H), 2.99 (ddd, J=6.0, 10.0, 16.4 Hz, 2H), 2.92-2.83 (m, 1H), 2.80 (t, J=5.6 Hz, 1H), 2.76 (t, J=5.6 Hz, 1H), 2.21 (d, J=11.2 Hz, 2H), 2.15-1.84 (m, 9H), 1.63 (s, 6H), 1.34-1.20 (m, 2H); LC-MS (ESI⁺) m/z 783.3 (M+H)⁺.

The following compounds in Table 4 where prepared according to the same procedure of Method 2 above.

TABLE 4
Compounds prepared by Method 2.

| | Inter- | Inter- | LCMS | |
|---|---|---|---|---|
| | mediate | mediate | (ES+) m/z | |
| I-# | Amine | Aldehyde | (M + H)⁺ | 1H NMR (400 MHz, DMSO-d6) δ |
| I-1 | WX | BOX | 739.6 | 11.09 (s, 1H), 10.67 (s, 1H), 8.24-8.21 (m, 2H), |
| | | | | 7.51 (s, 1H), 7.07 (d, J = 7.6 Hz, 1H), 6.99-6.93 |
| | | | | (m, 1H), 6.87 (d, J = 7.6 Hz, 1H), 6.24-6.11 (m, |
| | | | | 1H), 5.38 (dd, J = 5.6, 12.8 Hz, 1H), 4.42-4.30 |
| | | | | (m, 1H), 3.68 (s, 4H), 3.62 (s, 3H), 2.92-2.84 (m, |
| | | | | 4H), 2.71 (d, J = 13.2 Hz, 2H), 2.22 (d, J = 7.2 Hz, |
| | | | | 2H), 2.17-2.17 (m, 1H), 2.18 (s, 3H), 2.13-2.06 |
| | | | | (m, 3H), 1.98-1.85 (m, 6H), 1.61 (s, 7H), 1.43- |
| | | | | 1.35 (m, 2H), 1.11-1.01 (m, 2H), 0.86-0.78 (m, 4H) |
| I-2 | ATH | BBH | 853.5 | 11.10 (s, 1H), 10.51 (s, 1H), 8.68 (s, 1H), 8.47- |
| | | | | 8.37 (m, 2H), 8.33 (s, 1H), 8.22 (dd, J = 1.2, 7.6 |
| | | | | Hz, 1H), 7.59 (dd, J = 7.2, 8.4 Hz, 1H), 7.40 (s, |
| | | | | 1H), 7.05 (dd, J = 7.6, 17.6 Hz, 2H), 6.48 (t, J = |
| | | | | 5.6 Hz, 1H), 5.06 (dd, J = 5.6, 12.8 Hz, 1H), 4.40- |
| | | | | 4.27 (m, 1H), 4.13 (td, J = 3.2, 6.0 Hz, 1H), 3.31 |
| | | | | (s, 2H), 3.22 (q, J = 6.8 Hz, 2H), 3.12 (s, 2H), 3.02 |
| | | | | (s, 2H), 2.95-2.83 (m, 1H), 2.65-2.54 (m, 2H), |
| | | | | 2.24-2.18 (m, 4H), 2.14-2.02 (m, 3H), 1.93- |
| | | | | 1.79 (m, 4H), 1.79-1.71 (m, 2H), 1.65 (q, J = 6.8 |
| | | | | Hz, 2H), 1.31 (dd, J = 3.6, 7.8 Hz, 1H), 1.16-1.01 |
| | | | | (m, 2H), 0.97-0.90 (m, 2H), 0.80-0.74 (m, 2H) |
| I-3 | ATH | BOZ | 841.4 | 11.15-11.05 (m, 1H), 10.51 (s, 1H), 8.53 (s, 1H), |
| | | | | 8.49-8.38 (m, 2H), 8.23 (d, J = 7.6 Hz, 1H), 7.59 |
| | | | | (t, J = 7.6 Hz, 1H), 7.36-7.29 (m, 1H), 7.05 (dd, |
| | | | | J = 7.6, 18.4 Hz, 2H), 6.48 (br t, J = 6.0 Hz, 1H), |
| | | | | 5.06 (dd, J = 5.6, 12.8 Hz, 1H), 3.96 (s, 3H), 3.74 |
| | | | | (s, 3H), 3.22 (br d, J = 6.4 Hz, 2H), 3.18 (br s, 2H), |
| | | | | 3.08 (br s, 3H), 2.96-2.80 (m, 3H), 2.64-2.55 |
| | | | | (m, 2H), 2.26-2.18 (m, 4H), 2.06-2.00 (m, 1H), |
| | | | | 1.97-1.89 (m, 2H), 1.85-1.73 (m, 4H), 1.70- |
| | | | | 1.52 (m, 5H), 1.40-1.28 (m, 1H), 1.13-1.00 (m, 2H) |
| I-4 | AJF | ATF | 837.2 | 11.09 (s, 1H), 10.67 (s, 1H), 8.66 (s, 1H), 8.32- |
| | | | | 8.25 (m, 3H), 8.01 (dd, J = 2.4, 6.8 Hz, 1H), 7.58 |
| | | | | (dd, J = 12, 8.4 Hz, 1H), 7.14 (s, 1H), 7.10 (d, J = |
| | | | | 8.4 Hz, 1H), 7.02 (d, J = 7.2 Hz, 1H), 6.46 (t, J = |
| | | | | 5.6 Hz, 1H), 5.05 (dd, J = 5.4, 12.8 Hz, 1H), |
| | | | | 4.39-4.29 (m, 1H), 3.99 (s, 3H), 2.93-2.83 (m, |
| | | | | 1H), 2.63-2.52 (m, 4H), 2.31-2.23 (m, 2H), 2.23- |
| | | | | 1.98 (m, 11H), 1.94-1.83 (m, 6H), 1.57 (m, 3H), |
| | | | | 1.53-1.45 (m, 4H), 1.13-1.01 (m, 2H) |
| I-7 | ATC | BEO | 828.5 | 11.00 (s, 1H), 10.60 (s, 1H), 9.39 (dd, J = 1.2, 7.2 |
| | | | | Hz, 1H), 8.96 (dd, J = 1.2, 4.0 Hz, 1H), 8.73 (s, |
| | | | | 2H), 7.58 (t, J = 7.6 Hz, 1H), 7.42 (s, 1H), 7.36 |
| | | | | (dd, J = 4.0, 6.8 Hz, 1H), 7.04 (dd, J = 8.8, 19.6 |
| | | | | Hz, 2H), 6.47 (t, J = 5.2 Hz, 1H), 5.05 (dd, J = 5.2, |
| | | | | 12.8 Hz, 1H), 4.03 (s, 3H), 3.21 (d, J = 5.6 Hz, |
| | | | | 2H), 2.92-2.81 (m, 2H), 2.64-2.53 (m, 3H), 2.30- |
| | | | | 2.24 (m, 2H), 2.22-2.10 (m, 4H), 2.09-1.97 |
| | | | | (m, 3H), 1.95-1.81 (m, 4H), 1.73-1.64 (m, 2H), |
| | | | | 1.62-1.42 (m, 7H), 1.41-1.32 (m, 2H), 1.04- |
| | | | | 0.95 (m, 2H) |
| I-8 | BPA | ATF | 811.5 | 11.09 (s, 1H), 10.67 (s, 1H), 8.67 (s, 1H), 8.34- |
| | | | | 8.26 (m, 3H), 8.01 (dd, J = 1.8, 6.8 Hz, 1H), 7.59 |
| | | | | (t, J = 7.6 Hz, 1H), 7.15 (s, 1H), 7.09 (d, J = 8.4 |
| | | | | Hz, 1H), 7.02 (d, J = 7.2 Hz, 1H), 6.50 (t, J = 5.2 |
| | | | | Hz, 1H), 5.05 (dd, J = 5.2, 12.8 Hz, 1H), 4.41- |
| | | | | 4.30 (m, 1H), 3.99 (s, 3H), 2.84 (d, J = 9.2 Hz, |
| | | | | 3H), 2.63-2.51 (m, 4H), 2.23-2.10 (m, 7H), 2.07- |
| | | | | 1.99 (m, 1H), 1.96-1.82 (m, 6H), 1.69 (d, J = |
| | | | | 12 Hz, 2H), 1.61 (m, 1H), 1.52 (q, J = 6.4 Hz, 2H), |
| | | | | 1.41-1.28 (m, 1H), 1.26-1.16 (m, 2H), 1.15-1.02 |
| | | | | (m, 2H) |
| I-9 | ATC | ATJ | 855.4 | 11.10 (s, 1H), 10.50 (s, 1H), 9.94 (s, 1H), 8.69 (s, |
| | | | | 1H), 8.48-8.44 (m, 1H), 8.43-8.37 (m, 1H), 8.34 |
| | | | | (s, 1H), 8.21 (d, J = 7.6 Hz, 1H), 7.59 (t, J = 7.6 |
| | | | | Hz, 1H), 7.15 (s, 1H), 7.10-6.99 (m, 2H), 6.66- |
| | | | | 6.29 (m, 1H), 5.09-5.02 (m, 1H), 4.45-4.35 (m, |
| | | | | 1H), 3.98 (s, 3H), 3.41-3.28 (m, 2H), 3.23 (t, J = |
| | | | | 6.4 Hz, 2H), 2.93 (d, J = 5.2 Hz, 2H), 2.91-2.83 |
| | | | | (m, 2H), 2.82-2.71 (m, 1H), 2.65-2.51 (m, 2H), |
| | | | | 2.36-2.25 (m, 1H), 2.20-2.11 (m, 2H), 2.10- |
| | | | | 1.99 (m, 4H), 1.99-1.83 (m, 7H), 1.77-1.63 (m, |
| | | | | 3H), 1.58-1.50 (m, 1H), 1.46 (t, J = 9.6 Hz, 1H), |
| | | | | 1.31-1.17 (m, 2H) |
| I-10 | ATH | BPB | 827.6 | 11.17-11.00 (m, 1H), 10.18 (s, 1H), 9.39-9.27 |
| | | | | (m, 1H), 8.47-8.38 (m, 2H), 8.34 (s, 1H), 8.23 |
| | | | | (m, 1H), 7.65-7.52 (m, 2H), 7.11-6.97 (m, 3H), |
| | | | | 6.47 (t, J = 5.9 Hz, 1H), 5.05 (m, 1H), 3.98 (s, |
| | | | | 3H), 3.21 (m, 2H), 3.15-3.09 (m, 3H), 3.05-2.97 |
| | | | | (m, 2H), 2.94-2.80 (m, 1H), 2.63-2.55 (m, 1H), |
| | | | | 2.22-2.15 (m, 5H), 2.06-1.96 (m, 3H), 1.84-1.72 |
| | | | | (m, 4H), 1.67-1.57 (m, 2H), 1.44-1.18 (m, 4H), |
| | | | | 1.06-0.90 (m, 2H) |
| I-12 | ATH | BPC | 855.7 | 12.21 (s, 1H), 11.09 (s, 1H), 9.52 (s, 1H), 8.48- |
| | | | | 8.35 (m, 2H), 8.26-8.18 (m, 2H), 7.73 (s, 1H), |
| | | | | 7.58 (t, J = 7.9 Hz, 1H), 7.39 (s, 1H), 7.04 (dd, J = |
| | | | | 7.7, 18.7 Hz, 2H), 6.48 (m, 1H), 6.19-6.07 (m, |
| | | | | 1H), 5.05 (m, 1H), 3.22 (m, 4H), 2.95-2.81 (m, |
| | | | | 1H), 2.63-2.53 (m, 2H), 2.39-2.31 (m, 2H), 2.27- |
| | | | | 2.10 (m, 4H), 2.08-1.97 (m, 3H), 1.86-1.72 |
| | | | | (m, 4H), 1.69-1.55 (m, 9H), 1.46-1.22 (m, 4H), |
| | | | | 1.08-0.91 (m, 2H) |
| I-13 | AML | BAT | 799.5 | 11.26-10.90 (m, 1H), 10.53 (s, 1H), 9.37 (d, J = |
| | | | | 7.2 Hz, 1H), 8.95 (d, J = 2.0 Hz, 1H), 8.69 (d, J = |
| | | | | 10.0 Hz, 2H), 8.26 (s, 1H), 7.58 (t, J = 7.6 Hz, |
| | | | | 1H), 7.35 (d, J = 4.4 Hz, 1H), 7.09 (s, 1H), 7.06 |
| | | | | (d, J = 6.4 Hz, 1H), 6.99 (d, J = 8.8 Hz, 1H), 6.44 |
| | | | | (d, J = 6.0 Hz, 1H), 5.05 (dd, J = 5.2, 12.4 Hz, |
| | | | | 1H), 4.40-4.28 (m, 1H), 4.18-4.07 (m, 1H), 4.02 |
| | | | | (s, 3H), 2.96-2.82 (m, 1H), 2.65-2.55 (m, 2H), |
| | | | | 2.32 (d, J = 8.4 Hz, 4H), 2.26-2.07 (m, 6H), 2.06- |
| | | | | 1.98 (m, 1H), 1.96-1.81 (m, 4H), 1.72-1.52 |
| | | | | (m, 7H), 1.13-1.02 (m, 2H) |
| I-14 | ATH | BPD | 855.4 | 12.52-12.21 (m, 1H), 12.12-11.93 (m, 1H), |
| | | | | 11.24-10.81 (m, 1H), 8.60-8.52 (m, 1H), 8.50- |
| | | | | 8.41 (m, 1H), 8.36 (t, J = 7.6 Hz, 1H), 8.15 (d, J = |
| | | | | 7.6 Hz, 1H), 7.58 (t, J = 7.6 Hz, 1H), 7.52-7.27 |
| | | | | (m, 1H), 7.11-6.97 (m, 2H), 6.55-6.38 (m, 1H), |
| | | | | 5.83 (s, 1H), 5.05 (dd, J = 5.2, 12.8 Hz, 1H), 3.25- |
| | | | | 3.17 (m, 2H), 3.10 (s, 2H), 3.00 (s, 2H), 2.95- |
| | | | | 2.81 (m, 1H), 2.79-2.68 (m, 1H), 2.64-2.53 (m, |
| | | | | 2H), 2.24-2.17 (m, 4H), 2.17-2.11 (m, 1H), 2.08- |
| | | | | 1.97 (m, 3H), 1.89-1.79 (m, 2H), 1.79-1.71 |
| | | | | (m, 2H), 1.68-1.62 (m, 2H), 1.59 (s, 6H), 1.57- |
| | | | | 1.47 (m, 2H), 1.34-1.19 (m, 1H), 1.09-0.91 (m, 2H) |
| I-15 | ATC | BPF | 895.3 | 10.20 (s, 1H), 9.73 (d, J = 7.2 Hz, 1H), 8.93 (s, |
| | | | | 1H), 8.75 (s, 1H), 8.28 (s, 1H), 7.80 (d, J = 7.2 Hz, |
| | | | | 1H), 7.58-7.56 (m, 1H), 7.11 (s, 1H), 7.07 (d, J = |
| | | | | 8.4 Hz, 1H), 7.02 (d, J = 7.2 Hz, 1H), 6.51-6.45 |
| | | | | (m, 1H), 5.07-5.03 (m, 1H), 4.39-4.27 (m, 1H), |
| | | | | 3.98 (s, 3H), 3.24-3.21 (m, 2H), 2.94-2.82 (m, |
| | | | | 1H), 2.63-2.55 (m, 1H), 2.31-1.99 (m, 11H), |
| | | | | 1.95-1.82 (m, 6H), 1.73-1.65 (m, 2H), 1.61- |
| | | | | 1.51 (m, 3H), 1.47-1.45 (m, 2H), 1.41-1.36 (m, |
| | | | | 2H), 1.13-1.00 (m, 2H) |
| I-16 | AQK | BPG | 802.5 | 11.09 (s, 1H), 10.54 (s, 1H), 9.22-9.16 (m, 1H), |
| | | | | 8.73 (s, 1H), 8.53 (dd, J = 1.7, 8.4 Hz, 1H), 8.39 |
| | | | | (d, J = 8.1 Hz, 1H), 8.35 (s, 1H), 7.17 (s, 1H), 7.01- |
| | | | | 6.95 (m, 1H), 6.94-6.85 (m, 2H), 5.36 (dd, J = |
| | | | | 5.4, 12.7 Hz, 1H), 4.42-4.33 (m, 1H), 4.00 (s, |
| | | | | 3H), 3.65 (s, 3H), 3.16 (d, J = 11.0 Hz, 2H), 2.94- |
| | | | | 2.85 (m, 1H), 2.81-2.68 (m, 3H), 2.58-2.50 (m, |
| | | | | 1H), 2.36-2.25 (m 2H), 2.27 (s, 3H), 2.19-1.98 |
| | | | | (m, 5H), 1.96-1.40 (m, 10H), 1.20-1.02 (m, 2H) |
| I-17 | AZK | BMI | 788.3 | 11.10 (s, 1H), 10.58 (s, 1H), 8.66 (s, 1H), 8.50- |
| | | | | 8.46 (m, 1H), 8.45-8.40 (m, 1H), 8.26 (dd, J = |
| | | | | 0.9, 7.6 Hz, 1H), 7.38 (s, 1H), 7.05-6.97 (m, 3H), |
| | | | | 5.38 (dd, J = 5.3, 12.5 Hz, 1H), 4.44 (s, 2H), 4.03 |
| | | | | (s, 3H), 4.03-3.97 (m, 1H), 3.60 (s, 3H), 3.09 (d, |
| | | | | J = 6.4 Hz, 2H), 2.95-2.85 (m, 1H), 2.77-2.69 |
| | | | | (m, 1H), 2.66-2.60 (m, 1H), 2.55-2.51 (m, 1H), |
| | | | | 2.40-2.32 (m, 2H), 2.30-2.16 (m, 2H), 2.03- |
| | | | | 1.98 (m, 1H), 1.93 (d, J = 11.9 Hz, 2H), 1.87-1.77 |
| | | | | (m, 6H), 1.68-1.57 (m, 3H), 1.16-1.05 (m, 2H) |
| I-18 | AZK | BPI | 733.4 | 11.09 (s, 1H), 10.24 (d, J = 2.2 Hz, 1H), 8.54 (s, |
| | | | | 1H), 8.41 (m, 3H), 8.22 (d, J = 7.0 Hz, 1H), 7.58 |
| | | | | (d, J = 11.6 Hz, 1H), 7.06-6.92 (m, 3H), 5.36 (dd, |
| | | | | J = 4.8, 12.4 Hz, 1H), 5.09-4.97 (m, 1H), 3.58 (s, |
| | | | | 3H), 3.07-2.94 (m, 3H), 2.94-2.82 (m, 1H), 2.74- |
| | | | | 2.63 (m, 4H), 2.61-2.54 (m, 2H), 2.36-2.26 |
| | | | | (m, 3H), 2.22-2.11 (m, 2H), 2.04-1.94 (m, 1H), |
| | | | | 1.84-1.72 (m, 4H) |
| I-23 | BPP | BKQ | 775.5 | 11.07 (s, 1H), 10.23 (d, J = 2.0 Hz, 1H), 8.50 (s, |
| | | | | 1H), 8.47-8.30 (m, 4H), 8.22 (d, J = 7.6 Hz, 1H), |
| | | | | 7.54 (d, J = 12.0 Hz, 1H), 6.83 (d, J = 14.6 Hz, |
| | | | | 2H), 5.37-5.30 (m, 1H), 4.51-4.41 (m, 1H), 3.55 |
| | | | | (s, 3H), 3.21-3.12 (m, 2H), 2.98 (d, J = 10.4 Hz, |
| | | | | 2H), 2.94-2.84 (m, 1H), 2.74-2.59 (m, 2H), 2.29 |
| | | | | (s, 3H), 2.21 (d, J = 7.2 Hz, 2H), 2.16 (s, 1H), 2.09- |
| | | | | 2.03 (m, 2H), 2.01-1.94 (m, 4H), 1.93-1.88 |
| | | | | (m, 1H), 1.78 (s, 4H), 1.70-1.61 (m, 1H), 1.21- |
| | | | | 1.06 (m, 2H) |
| I-24 | AZK | BPQ | 777.5 | 11.11 (s, 1H), 10.53 (s, 1H), 8.63 (s, 1H), 8.54 (s, |
| | | | | 1H), 8.49-8.45 (m, 1H), 8.44-8.37 (m, 1H), 8.24 |
| | | | | (d, J = 7.8 Hz, 1H), 8.17 (s, 1H), 7.94 (s, 1H), 7.06- |
| | | | | 6.94 (m, 3H), 5.38 (dd, J = 5.4, 12.4 Hz, 1H), |
| | | | | 4.53-4.43 (m, 1H), 3.63-3.55 (m, 3H), 3.05- |
| | | | | 2.98 (m, 2H), 2.94-2.82 (m, 1H), 2.77-2.57 (m, |
| | | | | 3H), 2.28-2.06 (m, 6H), 2.03-1.87 (m, 5H), 1.86- |
| | | | | 1.62 (m, 5H), 1.22-1.08 (m, 2H) |
| I-25 | BPS | BKQ | 779.3 | 11.11 (s, 1H), 10.23 (d, J = 2.4 Hz, 1H), 8.50 (s, |
| | | | | 1H), 8.46-8.38 (m, 3H), 8.23 (dd, J = 0.8, 7.6 Hz, |
| | | | | 1H), 7.54 (d, J = 12.0 Hz, 1H), 7.06-6.99 (m, |
| | | | | 1H), 6.95-6.86 (m, 1H), 5.53 (dd, J = 5.2, 12.8 |
| | | | | Hz, 1H), 4.51-4.40 (m, 1H), 3.65 (s, 3H), 3.59- |
| | | | | 3.53 (m, 1H), 3.04 (d, J = 8.0 Hz, 2H), 2.64-2.57 |
| | | | | (m, 1H), 2.31-2.25 (m, 2H), 2.22-2.08 (m, 6H), |
| | | | | 2.00-1.91 (m, 4H), 1.86-1.66 (m, 6H), 1.19- |
| | | | | 1.08 (m, 2H) |
| I-26 | BPU | BKQ | 779.2 | 11.11 (s, 1H), 10.24 (d, J = 2.0 Hz, 1H), 8.50 (s, |
| | | | | 1H), 8.47-8.37 (m, 3H), 8.23 (d, J = 7.6 Hz, 1H), |
| | | | | 7.55 (d, J = 11.6 Hz, 1H), 7.04-6.96 (m, 1H), 6.85 |
| | | | | (dd, J = 2.4, 11.6 Hz, 1H), 5.36 (dd, J = 5.2, 12.8 |
| | | | | Hz, 1H), 4.50-4.41 (m, 1H), 3.57 (s, 3H), 3.23- |
| | | | | 3.23 (m, 1H), 2.98 (d, J = 10.8 Hz, 2H), 2.88-2.70 |
| | | | | (m, 2H), 2.65-2.57 (m, 1H), 2.24-2.14 (m, 4H), |
| | | | | 2.10-1.84 (m, 8H), 1.81-1.61 (m, 5H), 1.19- |
| | | | | 1.07 (m, 2H) |
| I-27 | AZK | BQE | 855.6 | 11.17-11.01 (m, 1H), 10.74 (s, 1H), 8.58 (s, 1H), |
| | | | | 8.52-8.47 (m, 1H), 8.43 (d, J = 8.0 Hz, 1H), 8.24 |
| | | | | (d, J = 6.8 Hz, 1H), 7.47 (s, 1H), 7.05-6.95 (m, |
| | | | | 3H), 5.41-5.33 (m, 1H), 4.62 (s, 1H), 4.43 (s, |
| | | | | 2H), 4.14-4.07 (m, 1H), 4.07-3.97 (m, 1H), 3.93 |
| | | | | (d, J = 7.6 Hz, 1H), 3.68 (d, J = 6.4 Hz, 1H), 3.59 |
| | | | | (s, 3H), 3.00-2.95 (m, 2H), 2.64-2.60 (m, 2H), |
| | | | | 2.18 (d, J = 6.0 Hz, 2H), 2.09-1.96 (m, 5H), 1.95- |
| | | | | 1.85 (m, 4H), 1.83-1.72 (m, 7H), 1.66-1.49 |
| | | | | (m, 4H), 1.13-1.00 (m, 2H) |
| I-28 | BAI | BOA | 844.4 | 11.23 (s, 1H), 11.09 (s, 1H), 8.73 (s, 1H), 8.54- |
| | | | | 8.49 (m, 1H), 8.46-8.40 (m, 1H), 8.29-8.25 (m, |
| | | | | 1H), 7.62 (s, 1H), 7.01-6.92 (m, 2H), 6.88 (d, J = |
| | | | | 7.2 Hz, 1H), 5.40-5.30 (m, 1H), 4.46 (s, 2H), |
| | | | | 4.06-3.99 (m, 1H), 3.87-3.84 (m, 4H), 3.63 (s, |
| | | | | 3H), 3.00-2.84 (m, 12H), 2.22 (d, J = 8.0 Hz, |
| | | | | 2H), 2.04-1.89 (m, 4H), 1.83-1.72 (m, 3H), 1.68- |
| | | | | 1.53 (m, 4H), 1.11-1.03 (m, 2H) |
| I-29 | AZK | BQF | 855.4 | 10.75 (s, 1H), 8.58 (s, 1H), 8.52-8.47 (m, 1H), |
| | | | | 8.44-8.42 (m, 1H), 8.25 (d, J = 6.8 Hz, 1H), 7.47 |
| | | | | (s, 1H), 7.06-6.93 (m, 3H), 5.42-5.32 (m, 1H), |
| | | | | 4.62 (s, 1H), 4.43 (s, 2H), 4.10 (s, 1H), 4.05-3.98 |
| | | | | (m, 1H), 3.93 (d, J = 8.0 Hz, 1H), 3.68 (d, J = 6.0 |
| | | | | Hz, 1H), 3.59 (s, 3H), 3.00-2.94 (m, 2H), 2.92- |
| | | | | 2.84 (m, 1H), 2.65-2.63 (m, 2H), 2.20-2.15 (m, 2H), |
| | | | | 2.09-2.00 (m, 4H), 1.95-1.85 (m, 4H), 1.84-1.69 |
| | | | | (m, 7H), 1.69-1.46 (m, 4H), 1.14-1.01 (m, 2H) |
| I-30 | AZK | BQH | 733.3 | 11.09 (s, 1H), 10.24 (s, 1H), 8.54 (s, 1H), 8.48- |
| | | | | 8.37 (m, 3H), 8.23 (d, J = 7.6 Hz, 1H), 7.59 (d, J = |
| | | | | 12.4 Hz, 1H), 7.08-6.93 (m, 3H), 5.41-5.34 (m, |
| | | | | 1H), 5.31-5.22 (m, 1H), 3.59 (s, 3H), 3.02 (d, J = |
| | | | | 11.2 Hz, 2H), 2.95-2.80 (m, 1H), 2.76-2.64 (m, 4H), |
| | | | | 2.61-2.56 (m, 3H), 2.40-2.29 (m, 3H), 2.21-2.09 |
| | | | | (m, 2H), 2.06-1.92 (m, 1H), 1.84-1.71 (m, 4H) |
| I-33 | AZK | BQI | 744.5 | 11.10 (s, 1H), 10.52 (s, 1H), 8.69 (s, 1H), 8.49- |
| | | | | 8.45 (m, 1H), 8.43-8.37 (m, 2H), 8.22 (d, J = 7.6 |
| | | | | Hz, 1H), 7.21 (s, 1H), 7.06-6.95 (m, 3H), 5.41- |
| | | | | 5.33 (m, 1H), 5.24-5.16 (m, 1H), 3.99 (s, 3H), |
| | | | | 3.59 (s, 3H), 3.05-3.01 (m, 2H), 2.90-2.87 (m, |
| | | | | 1H), 2.74-2.68 (m, 6H), 2.59 (br s, 2H), 2.39- |
| | | | | 2.34 (m, 2H), 2.17-2.10 (m, 2H), 2.01-1.97 (m, |
| | | | | 1H), 1.84-1.77 (m, 4H) |
| I-35 | BPP | ATJ | 787.3 | METHANOL-d₄) δ 8.76 (s, 1H), 8.47 (d, J = 8.0 |
| | | | | Hz, 1H), 8.34 (s, 1H), 8.31 (t, J = 8.4 Hz, 1H), |
| | | | | 8.20 (s, 1H), 7.80 (s, 1H), 7.08 (s, 1H), 6.92 (s, |
| | | | | 1H), 6.88 (s, 1H), 5.48-5.45 (m, 1H), 4.52-4.42 |
| | | | | (m, 1H), 4.06 (s, 3H), 3.78 (d, J = 10.4 Hz, 2H), |
| | | | | 3.72-3.62 (m, 4H), 3.30-3.22 (m, 2H), 3.21-3.12 (m, |
| | | | | 2H), 2.98-2.75 (m, 3H), 2.37 (s, 3H), 2.35-2.28 |
| | | | | (m, 2H), 2.24-2.01 (m, 10H), 1.51-1.36 (m, 2H) |
| I-36 | BQK | ATJ | 807.3 | 11.10 (s, 1H), 10.50 (s, 1H), 8.69 (s, 1H), 8.49- |
| | | | | 8.44 (m, 1H), 8.43-8.38 (m, 1H), 8.34 (s, 1H), |
| | | | | 8.22 (d, J = 7.6 Hz, 1H), 7.19 (s, 1H), 7.16 (s, 1H), |
| | | | | 7.01 (d, J = 1.6 Hz, 1H), 5.38 (dd, J = 5.6, 12.4 |
| | | | | Hz, 1H), 4.44-4.33 (m, 1H), 3.98 (s, 3H), 3.58 (s, |
| | | | | 3H), 3.22-3.18 (m, 1H), 2.98 (d, J = 10.4 Hz, |
| | | | | 2H), 2.91-2.79 (m, 1H), 2.78-2.70 (m, 1H), 2.68- |
| | | | | 2.59 (m, 1H), 2.21 (d, J = 6.4 Hz, 2H), 2.18- |
| | | | | 2.12 (m, 2H), 2.11-2.03 (m, 2H), 2.02-1.94 (m, |
| | | | | 3H), 1.94-1.85 (m, 2H), 1.84-1.73 (m, 4H), 1.70- |
| | | | | 1.57 (m, 1H), 1.13 (q, J = 11.2 Hz, 2H) |
| I-37 | BQL | ATJ | 807.2 | 11.12 (s, 1H), 10.51 (s, 1H), 8.69 (s, 1H), 8.49- |
| | | | | 8.44 (m, 1H), 8.43-8.38 (m, 1H), 8.34 (s, 1H), |
| | | | | 8.22 (dd, J = 1.2, 7.6 Hz, 1H), 7.15 (s, 1H), 7.13- |
| | | | | 7.04 (m, 2H), 5.40 (dd, J = 5.2, 12.8 Hz, 1H), 4.46- |
| | | | | 4.34 (m, 1H), 3.98 (s, 3H), 3.65 (s, 3H), 3.64- |
| | | | | 3.57 (m, 1H), 2.91-2.83 (m, 1H), 2.78-2.57 (m, |
| | | | | 5H), 2.53-2.51 (m, 3H), 2.17 (d, J = 10.8 Hz, |
| | | | | 2H), 2.05-1.87 (m, 6H), 1.87-1.69 (m, 3H), 1.27- |
| | | | | 1.12 (m, 2H) |
| I-38 | AZK | AYL | 828.3 | 11.09 (s, 2H), 8.79 (s, 1H), 8.52-8.48 (m, 1H), |
| | | | | 8.44-8.40 (m, 1H), 8.38 (s, 1H), 8.23 (dd, J = 0.8, |
| | | | | 7.8 Hz, 1H), 7.51 (s, 1H), 7.07-6.95 (m, 3H), 5.38 |
| | | | | (dd, J = 5.2, 12.4 Hz, 1H), 4.49-4.37 (m, 1H), |
| | | | | 3.93-3.81 (m, 4H), 3.60 (s, 3H), 3.23-3.02 (m, |
| | | | | 2H), 2.95-2.90 (m, 4H), 2.90-2.84 (m, 1H), 2.78- |
| | | | | 2.67 (m, 1H), 2.66-2.57 (m, 1H), 2.53-2.51 |
| | | | | (m, 1H), 2.42-2.23 (m, 2H), 2.22-2.10 (m, 3H), |
| | | | | 2.06-1.93 (m, 5H), 1.92 (s, 1H), 1.90-1.80 (m, |
| | | | | 4H), 1.78-1.64 (m, 1H), 1.27-1.09 (m, 2H) |
| I-39 | BQM | ATJ | 787.3 | 11.08 (s 1H), 10.50 (s, 1H), 8.69 (s, 1H), 8.48- |
| | | | | 8.45 (m, 1H), 8.43-8.38 (m, 1H), 8.34 (s, 1H), |
| | | | | 8.31 (s, 1H), 8.22 (dd, J = 0.8, 7.7 Hz, 1H), 7.16 |
| | | | | (s, 1H), 6.90-6.80 (m, 2H), 5.33 (dd, J = 5.6, 12.4 |
| | | | | Hz, 1H), 4.43-4.33 (m, 1H), 3.98 (s, 3H), 3.61 (s, |
| | | | | 3H), 3.00 (d, J = 10.8 Hz, 2H), 2.92-2.82 (m, |
| | | | | 1H), 2.75-2.68 (m, 1H), 2.64-2.59 (m, 1H), 2.46 |
| | | | | (s, 3H), 2.31-2.25 (m, 1H), 2.23-2.19 (m, 3H), |
| | | | | 2.18-2.12 (m, 2H), 2.07-1.85 (m, 8H), 1.71- |
| | | | | 1.65 (m, 2H), 1.21-1.08 (m, 2H) |
| I-40 | BQV | ATJ | 816.4 | 11.06 (s, 1H), 10.49 (s, 1H), 8.68 (s, 1H), 8.50- |
| | | | | 8.36 (m, 2H), 8.33 (s, 1H), 8.23-8.18 (M, 1H), |
| | | | | 7.15 (s, 1H), 6.89-6.80 (m, 1H), 6.78-6.69 (m, |
| | | | | 1H), 5.35-5.25 (m, 1H), 4.46-4.28 (m, 1H), 4.03 |
| | | | | (s, 3H), 3.64 (s, 3H), 3.27-3.24 (m, 2H), 3.10- |
| | | | | 2.98 (m, 2H), 2.90-2.79 (m, 1H), 2.72-2.63 (m, |
| | | | | 1H), 2.63-2.55 (m, 1H), 2.52-2.50 (m, 2H), 2.35 |
| | | | | (s, 3H), 2.31-2.23 (m, 4H), 2.19-2.08 (m, 2H), |
| | | | | 2.04-1.83 (m, 5H), 1.76-1.50 (m, 5H), 1.18- |
| | | | | 1.00 (m, 2H) |
| I-41 | AQK | BQW | 815.4 | 11.08 (s, 1H), 10.37 (s, 1H), 8.50 (s, 1H), 8.47- |
| | | | | 8.35 (m, 2H), 8.28-8.11 (m, 1H), 7.18 (s, 1H), |
| | | | | 7.04-6.84 (m, 3H), 6.17 (s, 1H), 5.40-5.28 (m, |
| | | | | 1H), 3.98 (s, 3H), 3.70 (s, 3H), 3.65 (s, 3H), 3.29 |
| | | | | (s, 3H), 3.23-3.15 (m, 2H), 2.93-2.85 (m, 1H), |
| | | | | 2.81-2.69 (m, 4H), 2.67-2.58 (m, 2H), 2.11- |
| | | | | 1.84 (m, 8H), 1.82-1.53 (m, 4H), 1.52-1.34 (m, |
| | | | | 2H), 1.21-1.05 (m, 2H) |
| I-42 | BQX | ATJ | 832.3 | 10.51 (s, 1H), 8.69 (s, 1H), 8.49-8.45 (m, 1H), |
| | | | | 8.44-8.39 (m, 1H), 8.35 (s, 1H), 8.29 (s, 1H), |
| | | | | 8.22 (dd, J = 1.0, 7.6 Hz, 1H), 7.16 (s, 1H), 6.86 |
| | | | | (d, J = 8.8 Hz, 1H), 6.68 (d, J = 8.8 Hz, 1H), 5.34- |
| | | | | 5.27 (m, 1H), 4.45-4.32 (m, 1H), 3.99 (s, 3H), |
| | | | | 3.78 (s, 3H), 3.63 (s, 3H), 2.99-2.82 (m, 6H), |
| | | | | 2.69-2.65 (m, 2H), 2.33 (d, J = 3.6 Hz, 2H), 2.30 |
| | | | | (d, J = 6.8 Hz, 2H), 2.26 (s, 3H), 2.19-2.12 (m, |
| | | | | 3H), 2.02-1.92 (m, 4H), 1.75-1.69 (m, 2H), 1.62- |
| | | | | 1.55 (m, 2H), 1.18-1.11 (m, 2H) |
| I-43 | AZK | BQW | 786.3 | 11.09 (s, 1H), 10.37 (s, 1H), 8.50 (s, 1H), 8.47- |
| | | | | 8.43 (m, 1H), 8.42-8.36 (m, 1H), 8.22-8.17 (m, |
| | | | | 1H), 7.18 (s, 1H), 7.08-6.92 (m, 3H), 6.17 (s, |
| | | | | 1H), 5.48-5.28 (m, 1H), 3.98 (s, 3H), 3.70 (s, |
| | | | | 3H), 3.60 (s, 3H), 3.30-3.28 (m, 2H), 3.17-3.01 |
| | | | | (m, 2H), 2.94-2.82 (m, 1H), 2.78-2.68 (m, 2H), |
| | | | | 2.66-2.56 (m, 2H), 2.07-1.91 (m, 6H), 1.90- |
| | | | | 1.76 (m, 4H), 1.75-1.56 (m, 2H), 1.52-1.39 (m, |
| | | | | 2H), 1.20-1.08 (m, 2H) |
| I-44 | AZK | BQY | 840.4 | 11.10 (s, 1H), 10.70 (s, 1H), 8.68 (s, 1H), 8.51- |
| | | | | 8.47 (m, 1H), 8.41 (t, J = 8.0 Hz, 1H), 8.35 (s, 1H), |
| | | | | 8.31-8.27 (m, 1H), 8.22 (dd, J = 0.8, 7.8 Hz, 1H), |
| | | | | 7.40 (s, 1H), 7.06-6.95 (m, 3H), 5.37 (dd, J = 5.2, |
| | | | | 12.8 Hz, 1H), 4.62 (s, 1H), 4.48-4.33 (m, 1H), |
| | | | | 4.06 (s, 1H), 4.01 (d, J = 7.6 Hz, 1H), 3.69-3.65 |
| | | | | (m, 1H), 3.60 (s, 3H), 3.00 (d, J = 11.2 Hz, 2H), |
| | | | | 2.95-2.84 (m, 2H), 2.78-2.70 (m, 1H), 2.69- |
| | | | | 2.64 (m, 2H), 2.61 (s, 1H), 2.35-2.33 (m, 1H), |
| | | | | 2.22 (d, J = 6.8 Hz, 2H), 2.17-2.13 (m, 2H), 2.12- |
| | | | | 2.04 (m, 3H), 2.03-1.87 (m, 6H), 1.86-1.76 |
| | | | | (m, 5H), 1.70-1.63 (m, 1H), 1.20-1.11 (m, 2H) |
| I-45 | AZK | BRB | 762.3 | 11.09 (s, 1H), 10.32 (s, 1H), 9.59 (s, 1H), 9.49 (s, |
| | | | | 1H), 8.51 (s, 1H), 8.24 (d, J = 7.6 Hz, 1H), 7.54 |
| | | | | (d, J = 12.0 Hz, 1H), 7.03-6.99 (m, 3H), 5.37 (dd, |
| | | | | J = 4.8, 11.6 Hz, 1H), 4.49-4.40 (m, 1H), 3.59 (s, |
| | | | | 3H), 3.03 (d, J = 11.2 Hz, 2H), 2.93-2.85 (m, 1H), |
| | | | | 2.72-2.64 (m, 2H), 2.27 (d, J = 7.2 Hz, 2H), 2.17 |
| | | | | (d, J = 10.4 Hz, 4H), 2.05-1.92 (m, 6H), 1.86- |
| | | | | 1.78 (m, 4H), 1.71-1.67 (m, 1H), 1.20-1.11 (m, 2H) |
| I-47 | BRE | BKQ | 791.3 | 11.08 (s, 1H), 10.24 (d, J = 2.1 Hz, 1H), 8.51 (s, |
| | | | | 1H), 8.47-8.38 (m, 3H), 8.24-8.21 (m, 1H), 7.55 |
| | | | | (d, J = 11.9 Hz, 1H), 6.91 (d, J = 8.4 Hz, 1H), 6.72 |
| | | | | (d, J = 8.8 Hz, 1H), 5.39-5.27 (m, 1H), 4.52- |
| | | | | 4.39 (m, 1H), 3.77 (s, 3H), 3.56 (s, 3H), 2.97 (d, J = |
| | | | | 11.2 Hz, 2H), 2.71-2.62 (m, 3H), 2.22-2.13 |
| | | | | (m, 6H), 2.02-1.94 (m, 7H), 1.71-1.54 (m, 4H), |
| | | | | 1.20-1.10 (m, 2H) |
| I-48 | BRE | ATJ | 803.3 | 11.08 (s, 1H), 10.51 (s, 1H), 8.70 (s, 1H), 8.49- |
| | | | | 8.45 (m, 1H), 8.44-8.39 (m, 1H), 8.34 (s, 1H), |
| | | | | 8.22 (d, J = 7.6 Hz, 1H), 7.17 (s, 1H), 6.91 (d, J = |
| | | | | 8.4 Hz, 1H), 6.72 (d, J = 8.8 Hz, 1H), 5.33 (dd, J = |
| | | | | 4.4, 12.4 Hz, 1H), 4.39 (t, J = 11.6 Hz, 1H), 3.99 |
| | | | | (s, 3H), 3.77 (s, 3H), 3.57 (s, 3H), 2.97 (d, J = 10.0 |
| | | | | Hz, 2H), 2.92-2.86 (m, 1H), 2.71-2.59 (m, 2H), |
| | | | | 2.41-2.34 (m, 4H), 2.22-2.15 (m, 4H), 2.00- |
| | | | | 1.91 (m, 6H), 1.66 (d, J = 1.4 Hz, 1H), 1.61-1.55 |
| | | | | (m, 2H), 1.19-1.10 (m, 2H) |
| I-49 | AZK | BRC | 694.2 | 11.13-11.05 (m, 1H), 10.40-10.24 (m, 1H), 9.40- |
| | | | | 9.29 (m, 1H), 9.00-8.81 (m, 2H), 8.55-8.43 |
| | | | | (m, 1H), 8.39-8.28 (m, 1H), 7.61-7.47 (m, 1H), |
| | | | | 7.10-6.92 (m, 3H), 5.43-5.34 (m, 1H), 4.55- |
| | | | | 4.39 (m, 1H), 3.59 (s, 3H), 3.04-3.00 (m, 2H), |
| | | | | 2.95-2.85 (m, 2H), 2.71-2.64 (m, 2H), 2.26- |
| | | | | 2.23 (m, 2H), 2.12-2.07 (m, 2H), 2.13-2.09 (m, |
| | | | | 2H), 2.03-1.93 (m, 6H), 1.84-1.75 (m, 4H), 1.21- |
| | | | | 1.11 (m, 2H) |
| I-51 | BPS | ATJ | 791.2 | 11.10 (s, 1H), 10.50 (s, 1H), 8.69 (s, 1H), 8.49- |
| | | | | 8.44 (m, 1H), 8.43-8.37 (m, 1H), 8.33 (s, 1H), |
| | | | | 8.21 (d, J = 8.4 Hz, 1H), 7.16 (s, 1H), 7.07-6.99 |
| | | | | (m, 1H), 6.94-6.78 (m, 1H), 5.59-5.23 (m, 1H), |
| | | | | 4.43-4.30 (m, 1H), 3.98 (s, 3H), 3.64 (s, 2H), |
| | | | | 3.58 (d, J = 7.2 Hz, 1H), 3.23-3.13 (m, 2H), 3.04- |
| | | | | 2.92 (m, 3H), 2.65-2.55 (m, 1H), 2.21 (d, J = |
| | | | | 7.0 Hz, 2H), 2.18-2.12 (m, 2H), 2.11-1.94 (m, |
| | | | | 5H), 1.93-1.84 (m, 2H), 1.83-1.57 (m, 5H), 1.21- |
| | | | | 1.03 (m, 2H) |
| I-52 | BPU | ATJ | 791.3 | 11.10 (s, 1H), 10.50 (s, 1H), 8.69 (s, 1H), 8.49- |
| | | | | 8.44 (m, 1H), 8.43-8.37 (m, 1H), 8.34 (s, 1H), |
| | | | | 8.22 (d, J = 7.2 Hz, 1H), 7.16 (s, 1H), 7.02 (d, J = |
| | | | | 8.8 Hz, 1H), 6.89-6.74 (m, 1H), 5.36 (dd, J = 5.6, |
| | | | | 12.4 Hz, 1H), 4.46-4.33 (m, 1H), 3.98 (s, 3H), |
| | | | | 3.59 (s, 3H), 3.27-3.05 (m, 4H), 2.91-2.70 (m, |
| | | | | 2H), 2.65-2.57 (m, 1H), 2.46-2.34 (m, 3H), 2.20- |
| | | | | 2.14 (m, 2H), 2.02-1.71 (m, 10H), 1.27-1.10 |
| | | | | (m, 2H) |
| I-53 | BOK | ATJ | 836.3 | 11.11 (s, 1H), 10.50 (s, 1H), 8.69 (s, 1H), 8.49- |
| | | | | 8.44 (m, 1H), 8.43-8.37 (m, 1H), 8.34 (s, 1H), |
| | | | | 8.23-8.20 (m, 1H), 7.16 (s, 1H), 7.06-6.97 (m, |
| | | | | 2H), 5.37 (dd, J = 5.2, 12.8 Hz, 1H), 4.44-4.30 |
| | | | | (m, 1H), 3.98 (s, 3H), 3.65 (s, 3H), 3.54 (t, J = |
| | | | | 10.8 Hz, 2H), 3.02 (d, J = 10.8 Hz, 2H), 2.92- |
| | | | | 2.81 (m, 1H), 2.72-2.57 (m, 2H), 2.45-2.36 (m, |
| | | | | 1H), 2.30 (d, J = 6.8 Hz, 2H), 2.25 (s, 3H), 2.15 |
| | | | | (d, J = 11.6 Hz, 2H), 2.04-1.86 (m, 5H), 1.76- |
| | | | | 1.50 (m, 5H), 1.17-1.04 (m, 2H) |
| I-54 | BQQ | ATJ | 836.2 | 11.09 (s, 1H), 10.50 (s, 1H), 8.69 (s, 1H), 8.49- |
| | | | | 8.44 (m, 1H), 8.43-8.37 (m, 1H), 8.34 (s, 1H), |
| | | | | 8.21 (dd, J = 0.8, 7.6 Hz, 1H), 7.15 (s, 1H), 7.09 |
| | | | | (s, 1H), 6.91 (d, J = 2.0 Hz, 1H), 5.36 (dd, J = 5.2, |
| | | | | 12.4 Hz, 1H), 4.44-4.32 (m, 1H), 3.98 (s, 3H), |
| | | | | 3.62 (s, 3H), 3.17 (d, J = 10.2 Hz, 2H), 2.89-2.81 |
| | | | | (m, 1H), 2.79-2.69 (m, 3H), 2.65-2.54 (m, 2H), |
| | | | | 2.47-2.22 (m, 5H), 2.19-2.12 (m, 2H), 2.02- |
| | | | | 1.81 (m, 7H), 1.77-1.55 (m, 3H), 1.21-1.05 (m, 2H) |
| I-55 | AZK | BAS | 840.3 | 11.09 (s, 1H), 10.69 (s, 1H), 8.68 (s, 1H), 8.51- |
| | | | | 8.46 (m, 1H), 8.41 (t, J = 7.6 Hz, 1H), 8.34 (s, 1H), |
| | | | | 8.21 (dd, J = 0.8, 7.6 Hz, 1H), 7.39 (s, 1H), 7.05- |
| | | | | 6.93 (m, 3H), 5.37 (dd, J = 5.2, 12.4 Hz, 1H), 4.61 |
| | | | | (s, 1H), 4.46-4.32 (m, 1H), 4.05 (s, 1H), 4.00 (d, |
| | | | | J = 7.6 Hz, 1H), 3.66 (dd, J = 1.6, 7.6 Hz, 1H), |
| | | | | 3.59 (s, 3H), 3.44-3.41 (m, 1H), 3.26-3.22 (m, |
| | | | | 2H), 3.04-2.96 (m, 2H), 2.93-2.84 (m, 1H), 2.77- |
| | | | | 2.61 (m, 2H), 2.21 (d, J = 6.8 Hz, 2H), 2.18- |
| | | | | 2.04 (m, 5H), 2.03-1.88 (m, 5H), 1.87-1.75 (m, |
| | | | | 5H), 1.68-1.61 (m, 1H), 1.13 (q, J = 11.6 Hz, 2H) |
| I-56 | BRS | ATJ | 816.3 | 11.06 (s, 1H), 10.50 (s, 1H), 8.69 (s, 1H), 8.49- |
| | | | | 8.44 (m, 1H), 8.44-8.37 (m, 1H), 8.34 (s, 1H), |
| | | | | 8.22 (dd, J = 1.2, 7.6 Hz, 1H), 7.16 (s, 1H), 6.71 |
| | | | | (s, 2H), 5.32 (dd, J = 5.2, 12.8 Hz, 1H), 4.44-4.31 |
| | | | | (m, 1H), 3.98 (s, 3H), 3.61 (s, 3H), 3.16-3.12 (m, |
| | | | | 2H), 2.93-2.84 (m, 1H), 2.76-2.61 (m, 4H), 2.45- |
| | | | | 2.40 (m, 1H), 2.32-2.28 (m, 2H), 2.28 (s, 3H), |
| | | | | 2.25 (s, 3H), 2.18-2.11 (m, 2H), 2.00-1.84 (m, |
| | | | | 5H), 1.82-1.75 (m, 2H), 1.71-1.50 (m, 3H), 1.18- |
| | | | | 1.03 (m, 2H) |
| I-57 | AZK | BRP | 743.4 | 11.09 (s, 1H), 10.36 (s, 1H), 8.43-8.33 (m, 3H), |
| | | | | 8.30 (d, J = 1.2 Hz, 1H), 8.20-8.13 (m, 1H), 7.64- |
| | | | | 7.59 (m, 1H), 7.58-7.52 (m, 1H), 7.05-6.95 |
| | | | | (m, 3H), 5.37 (dd, J = 5.4, 12.4 Hz, 1H), 4.51- |
| | | | | 4.39 (m, 1H), 3.59 (s, 3H), 3.06-3.03 (m, 3H), |
| | | | | 2.94-2.83 (m, 1H), 2.77-2.68 (m, 1H), 2.66- |
| | | | | 2.57 (m, 1H), 2.29-2.27 (m, 2H), 2.18-2.16 (m, |
| | | | | 4H), 2.04-1.94 (m, 4H), 1.94-1.88 (m, 1H), 1.81- |
| | | | | 1.78 (m, 3H), 1.75-1.63 (m, 1H), 1.23-1.08 |
| | | | | (m, 2H) |
| I-58 | AQK | BRP | 772.3 | 11.09 (s, 1H), 10.36 (s, 1H), 8.42-8.33 (m, 3H), |
| | | | | 8.29 (d, J = 1.2 Hz, 1H), 8.20-8.14 (m, 1H), 7.64- |
| | | | | 7.59 (m, 1H), 7.58-7.52 (m, 1H), 7.01-6.95 |
| | | | | (m, 1H), 6.94-6.84 (m, 2H), 5.35 (dd, J = 5.2, |
| | | | | 12.4 Hz, 1H), 4.50-4.39 (m, 1H), 3.64 (s, 3H), |
| | | | | 3.16 (d, J = 12.0 Hz, 3H), 2.95-2.83 (m, 1H), 2.78- |
| | | | | 2.60 (m, 4H), 2.34-2.29 (m, 2H), 2.27 (s, 3H), |
| | | | | 2.16 (m, 2H), 2.04-1.89 (m, 5H), 1.80 (m 2H), |
| | | | | 1.73-1.53 (m, 3H), 1.18-1.05 (m, 2H) |
| I-59 | AQK | BRQ | 803.6 | 11.09 (s, 1H), 10.14 (s, 1H), 8.47-8.34 (m, 2H), |
| | | | | 8.21 (d, J = 8.4 Hz, 1H), 8.07 (d, J = 7.6 Hz, 1H), |
| | | | | 7.46 (d, J = 12.0 Hz, 1H), 7.02-6.85 (m, 3H), 6.25 |
| | | | | (s, 1H), 5.39-5.31 (m, 1H), 3.69 (s, 3H), 3.64 (s, |
| | | | | 3H), 3.17 (d, J = 10.0 Hz, 3H), 2.96-2.84 (m, |
| | | | | 2H), 2.82-2.69 (m, 5H), 2.62-2.58 (s, 2H), 2.10- |
| | | | | 1.90 (m, 6H), 1.89-1.81 (m, 2H), 1.77-1.64 |
| | | | | (m, 3H), 1.62-1.53 (m, 1H), 1.51-1.38 (m, 2H), |
| | | | | 1.17-1.05 (m, 2H) |
| I-60 | BCD | BGF | 751.4 | 11.50 (s, 1H), 8.59 (s, 1H), 8.54 (s, 1H), 8.33 (s, |
| | | | | 1H), 8.19 (s, 1H), 7.53 (s, 1H), 7.07-6.94 (m, |
| | | | | 3H), 6.08 (s, 1H), 5.44 (dd, J = 5.2, 12.8 Hz, 1H), |
| | | | | 4.47-4.35 (m, 1H), 3.59 (s, 3H), 3.03 (s, 3H), |
| | | | | 3.02-2.91 (m, 4H), 2.82-2.64 (m, 3H), 2.22 (d, |
| | | | | J = 6.8 Hz, 2H), 2.18-2.05 (m, 4H), 2.04-1.86 |
| | | | | (m, 6H), 1.85-1.73 (m, 4H), 1.70-1.64 (m, 1H), |
| | | | | 1.61 (s, 6H), 1.21-1.05 (m, 2H) |
| I-61 | BFL | BGF | 780.8 | 11.49 (s, 1H), 8.58 (s, 1H), 8.54 (s, 1H), 8.33 (s, |
| | | | | 1H), 7.53 (s, 1H), 7.02-6.83 (m, 3H), 6.07 (s, |
| | | | | 1H), 5.41 (dd, J = 5.2, 12.8 Hz, 1H), 4.46-4.37 |
| | | | | (m, 1H), 3.65 (s, 3H), 3.19-3.13 (m, 2H), 3.03 (s, |
| | | | | 3H), 2.98-2.95 (m, 1H), 2.81-2.71 (m, 6H), 2.30 |
| | | | | (d, J = 6.8 Hz, 2H), 2.26 (s, 3H), 2.17-2.11 (m, |
| | | | | 2H), 2.04-1.85 (m, 7H), 1.83-1.77 (m, 2H), 1.71- |
| | | | | 1.65 (m, 2H), 1.61 (s, 6H), 1.58-1.53 (m, 1H), |
| | | | | 1.17-1.06 (m, 2H) |
| I-62 | AZK | BRR | 744.3 | 11.09 (s, 1H), 10.20 (s, 1H), 9.06 (s, 1H), 8.61 (s, |
| | | | | 1H), 8.49-8.47 (m, 2H), 8.41 (t, J = 7.6 Hz, 1H), |
| | | | | 8.23 (d, J = 7.6 Hz, 1H), 7.04-6.96 (m, 3H), 5.37 |
| | | | | (dd, J = 5.2, 12.4 Hz, 1H), 4.60-4.55 (m, 1H), |
| | | | | 3.59 (s, 3H), 3.22 (s, 1H), 3.01 (d, J = 10.4 Hz, |
| | | | | 2H), 2.93-2.84 (m, 1H), 2.75-2.59 (m, 2H), 2.24- |
| | | | | 2.18 (m, 4H), 2.12-2.07 (m, 2H), 2.01-1.92 |
| | | | | (m, 5H), 1.80-1.69 (m, 5H), 1.23-1.11 (m, 2H) |
| I-63 | AZK | BRV | 733.6 | 11.09 (s, 1H), 10.18 (d, J = 3.2 Hz, 1H), 9.39 (d, J = |
| | | | | 7.2 Hz, 1H), 8.97-8.89 (m, 1H), 8.74 (s, 1H), |
| | | | | 8.66 (d, J = 7.6 Hz, 1H), 8.46 (s, 1H), 8.31 (br s, |
| | | | | 1H), 7.53 (d, J = 12.0 Hz, 1H), 7.36 (dd, J = 4.2, |
| | | | | 6.8 Hz, 1H), 7.06-6.92 (m, 3H), 5.39-5.34 (m, |
| | | | | 1H), 4.48-4.37 (m, 1H), 3.59 (s, 3H), 3.00-2.99 |
| | | | | (m, 2H), 2.94-2.83 (m, 2H), 2.73-2.64 (m, 2H), |
| | | | | 2.23-2.13 (m, 4H), 2.04-1.86 (m, 6H), 1.85- |
| | | | | 1.74 (m, 4H), 1.70-1.61 (m, 1H), 1.19-1.08 (m, 2H) |
| I-64 | BRY | AGL | 783.3 | 12.37 (s, 1H), 10.54 (s, 1H), 9.26 (s, 1H), 8.72 (s, |
| | | | | 1H), 8.48 (s, 1H), 8.45 (d, J = 7.6 Hz, 1H), 8.37 |
| | | | | (t, J = 7.6 Hz, 2H), 8.16 (d, J = 7.6 Hz, 1H), 8.06 |
| | | | | (s, 1H), 7.92 (d, J = 8.8 Hz, 1H), 7.79 (dd, J = 1.4, |
| | | | | 8.8 Hz, 1H), 7.58 (s, 1H), 5.94 (s, 1H), 4.47-4.41 |
| | | | | (m, 1H), 3.97-3.90 (m, 1H), 3.76-3.70 (m, 1H), |
| | | | | 3.08-3.04 (m, 2H), 3.01-2.92 (m, 1H), 2.79- |
| | | | | 2.71 (m, 2H), 2.29-2.25 (m, 2H), 2.21-2.07 (m, |
| | | | | 4H), 2.03-1.76 (m, 8H), 1.71-1.67 (m, 1H), 1.62 |
| | | | | (s, 6H), 1.24-1.08 (m, 2H) |
| I-65 | BRZ | AGL | 812.5 | 12.35 (s, 1H), 10.50 (d, J = 3.6 Hz, 1H), 9.07 (d, |
| | | | | J = 3.6 Hz, 1H), 8.71 (d, J = 4.0 Hz, 1H), 8.45- |
| | | | | 8.43 (m, 1H), 8.41-8.32 (m, 2H), 8.26 (d, J = 4.4 |
| | | | | Hz, 1H), 8.19-8.13 (m, 1H), 7.79-7.76 (m, 1H), |
| | | | | 7.67-7.65 (m, 1H), 7.57 (d, J = 3.6 Hz, 1H), 7.42 |
| | | | | (s, 1H), 5.94 (d, J = 4.4 Hz, 1H), 4.44-4.38 (m, |
| | | | | 1H), 4.02-3.95 (m, 2H), 3.92-3.87 (m, 1H), 3.71- |
| | | | | 3.67 (m, 1H), 2.99-2.90 (m, 2H), 2.77-2.66 |
| | | | | (m, 4H), 2.34-2.29 (m, 2H), 2.24 (s, 3H), 2.16- |
| | | | | 2.10 (m, 2H), 1.99-1.88 (m, 4H), 1.84-1.67 (m, |
| | | | | 2H), 1.65-1.56 (m, 8H), 1.16-1.05 (m, 2H) |
| I-66 | BSA | AGL | 812.3 | 12.37 (s, 1H), 10.53 (s, 1H), 9.43 (s, 1H), 8.72 (s, |
| | | | | 1H), 8.53 (s, 1H), 8.48-8.43 (m, 1H), 8.39-8.34 |
| | | | | (m, 2H), 8.16 (d, J = 8.0 Hz, 1H), 7.74-7.68 (m, |
| | | | | 1H), 7.62-7.56 (m, 2H), 7.26 (d, J = 7.6 Hz, 1H), |
| | | | | 5.94 (s, 1H), 4.50-4.38 (m, 1H), 3.93-3.87 (m, |
| | | | | 1H), 3.73-3.67 (m, 1H), 3.49-3.42 (m, 2H), 3.00- |
| | | | | 2.92 (m, 1H), 2.90-2.80 (m, 2H), 2.78-2.71 |
| | | | | (m, 1H), 2.60-2.51 (m, 2H), 2.36 (d, J = 6.8 Hz, |
| | | | | 2H), 2.31 (s, 3H), 2.16 (d, J = 12.0 Hz, 2H), 2.05- |
| | | | | 1.81 (m, 8H), 1.62 (s, 6H), 1.22-1.06 (m, 2H) |
| I-67 | BQM | BKQ | 775.5 | 11.08 (s, 1H), 10.23 (d, J = 2.4 Hz, 1H), 8.50 (s, |
| | | | | 1H), 8.47-8.35 (m, 3H), 8.23 (dd, J = 1.2, 8.0 Hz, |
| | | | | 1H), 7.55 (d, J = 12.0 Hz, 1H), 6.92-6.77 (m, |
| | | | | 2H), 5.34 (dd, J = 5.2, 12.4 Hz, 1H), 4.53-4.39 |
| | | | | (m, 1H), 3.61 (s, 3H), 3.50-3.43 (m, 1H), 3.02 (d, |
| | | | | J = 11.2 Hz, 2H), 2.93-2.82 (m, 1H), 2.74-2.58 |
| | | | | (m, 2H), 2.46 (s, 3H), 2.30-2.14 (m, 6H), 2.10- |
| | | | | 1.89 (m, 7H), 1.69 (d, J = 10.0 Hz, 3H), 1.21-1.07 |
| | | | | (m, 2H) |
| I-68 | BSN | BRP | 725.5 | 10.54 (s, 1H), 10.36 (s, 1H), 9.63 (s, 1H), 8.57 (s, |
| | | | | 1H), 8.45-8.32 (m, 3H), 8.30 (s, 1H), 8.24-8.08 |
| | | | | (m, 1H), 7.90-7.84 (m, 1H), 7.79 (t, J = 7.2 Hz, |
| | | | | 1H), 7.69-7.58 (m, 2H), 7.58-7.50 (m, 1H), 4.53- |
| | | | | 4.42 (m, 1H), 3.92 (m, 1H), 3.76-3.65 (m, 2H), |
| | | | | 3.21-3.11 (m, 3H), 2.98 (s, 1H), 2.76 (m, 1H), |
| | | | | 2.47-2.34 (m, 3H), 2.25-2.13 (m, 2H), 2.09- |
| | | | | 1.84 (m, 8H), 1.82-1.71 (m, 1H), 1.27-1.11 (m, 2H) |
| I-69 | AZK | BSB | 783.4 | 11.09 (s, 1H), 10.74 (s, 1H), 8.68-8.61 (m, 1H), |
| | | | | 8.53-8.46 (m, 1H), 8.45-8.36 (m, 2H), 8.24- |
| | | | | 8.22 (m, 1H), 7.49 (s, 1H), 7.06-6.94 (m, 3H), |
| | | | | 5.37 (dd, J = 5.2, 12.4 Hz, 1H), 4.49-4.36 (m, |
| | | | | 1H), 3.59 (s, 3H), 3.24-3.18 (m, 1H), 2.99 (d, J = |
| | | | | 11.2 Hz, 2H), 2.95-2.81 (m, 1H), 2.77-2.68 |
| | | | | (m, 1H), 2.62 (d, J = 17.6 Hz, 1H), 2.21 (d, J = 6.8 |
| | | | | Hz, 2H), 2.18-2.12 (m, 2H), 2.10-2.04 (m, 2H), |
| | | | | 2.03-1.86 (m, 6H), 1.85-1.73 (m, 4H), 1.70- |
| | | | | 1.60 (m, 1H), 1.20-1.05 (m, 4H), 0.81-0.74 (m, 2H) |
| I-70 | AZK | BSC | 757.4 | 11.10 (s, 1H), 10.13 (s, 1H), 8.47-8.35 (m, 3H), |
| | | | | 8.24-8.13 (m, 2H), 7.51 (s, 1H), 7.11-6.93 (m, |
| | | | | 3H), 5.38 (dd, J = 5.2, 12.8 Hz, 1H), 4.53-4.40 |
| | | | | (m, 1H), 3.61 (s, 3H), 3.30 (s, 4H), 2.92-2.85 (m, |
| | | | | 1H), 2.76-2.58 (m, 3H), 2.41 (s, 3H), 2.19 (d, J = |
| | | | | 10.4 Hz, 3H), 2.10-1.67 (m, 11H), 1.31-1.16 (m, 2H) |
| I-72 | BQK | BKQ | 795.4 | 11.10 (s, 1H), 10.23 (d, J = 2.0 Hz, 1H), 8.50 (s, |
| | | | | 1H), 8.47-8.36 (m, 3H), 8.22 (d, J = 7.6 Hz, 1H), |
| | | | | 7.54 (d, J = 12.0 Hz, 1H), 7.19 (s, 1H), 7.01 (d, J = |
| | | | | 1.6 Hz, 1H), 5.38 (dd, J = 5.2, 12.4 Hz, 1H), |
| | | | | 4.52-4.39 (m, 1H), 3.58 (s, 3H), 2.98 (d, J = 10.8 |
| | | | | Hz, 2H), 2.90-2.57 (m, 4H), 2.24-2.12 (m, 4H), 2.11- |
| | | | | 1.88 (m, 8H), 1.85-1.72 (m, 4H), 1.21-1.09 (m, 2H) |
| I-73 | BQM | BPQ | 791.3 | 11.08 (s, 1H), 10.52 (s, 1H), 8.64 (s, 1H), 8.54 (s, |
| | | | | 1H), 8.49-8.39 (m, 2H), 8.24 (dd, J = 0.8, 7.6 Hz, |
| | | | | 1H), 7.94 (s, 1H), 6.94-6.82 (m, 2H), 5.35 (dd, J = |
| | | | | 5.2, 12.4 Hz, 1H), 4.58-4.45 (m, 1H), 3.64 (s, |
| | | | | 3H), 3.59 (d, J = 14.4 Hz, 1H), 2.92-2.84 (m, |
| | | | | 1H), 2.76-2.58 (m, 3H), 2.52 (s, 2H), 2.47 (s, |
| | | | | 3H), 2.47-2.34 (m, 3H), 2.20 (d, J = 10.0 Hz, 3H), |
| | | | | 2.05-1.91 (m, 6H), 1.90-1.72 (m, 3H), 1.35-1.12 (m, 2H) |
| I-74 | BSP | BRP | 761.3 | 11.12-11.08 (m, 1H), 10.36 (s, 1H), 8.42-8.33 |
| | | | | (m, 3H), 8.30 (d, J = 1.2 Hz, 1H), 8.19-8.15 (m, |
| | | | | 1H), 7.64-7.60 (m, 1H), 7.57-7.53 (m, 1H), 7.08- |
| | | | | 6.97 (m, 1H), 6.92-6.83 (m, 1H), 5.42-5.34 |
| | | | | (m, 1H), 4.51-4.40 (m, 1H), 3.59 (s, 2H), 3.36 (s, |
| | | | | 1H), 3.29 (s, 3H), 3.15-2.96 (m, 2H), 2.93-2.82 |
| | | | | (m, 1H), 2.72-2.60 (m, 3H), 2.22-2.13 (m, 4H), |
| | | | | 2.10 (d, J = 1.6 Hz, 1H), 2.04-1.93 (m, 6H), 1.80- |
| | | | | 1.74 (m, 1H), 1.73-1.62 (m, 1H), 1.21-1.11 (m, 2H) |
| I-75 | BRE | BRP | 772.8 | 11.09 (s, 1H), 10.37 (s, 1H), 8.42 (s, 1H), 8.41- |
| | | | | 8.33 (m, 2H), 8.31 (d, J = 1.3 Hz, 1H), 8.20-8.14 |
| | | | | (m, 1H), 7.65-7.61 (m, 1H), 7.58-7.54 (m, 1H), |
| | | | | 6.93 (d, J = 8.6 Hz, 1H), 6.74 (d, J = 8.8 Hz, 1H), |
| | | | | 5.33 (dd, J = 5.2, 12.8 Hz, 1H), 4.53-4.42 (m, |
| | | | | 1H), 3.79 (s, 3H), 3.58 (s, 3H), 3.19-3.09 (m, |
| | | | | 2H), 2.94-2.83 (m, 1H), 2.76-2.57 (m, 3H), 2.49- |
| | | | | 2.42 (m, 4H), 2.25-2.15 (m, 3H), 2.05-1.89 |
| | | | | (m, 6H), 1.82-1.71 (m, 1H), 1.66 (d, J = 10.4 Hz, |
| | | | | 2H), 1.25-1.14 (m, 2H) |
| I-76 | AZK | BSQ | 811.3 | 11.09 (s, 1H), 10.29 (s, 1H), 8.65 (s, 1H), 8.47- |
| | | | | 8.39 (m, 3H), 8.24 (dd, J = 1.2, 7.6 Hz, 1H), 8.19 |
| | | | | (s, 1H), 7.06-6.95 (m, 3H), 5.37 (dd, J = 5.6, 12.4 |
| | | | | Hz, 1H), 4.61-4.53 (m, 1H), 3.59 (s, 3H), 3.00 (d, |
| | | | | J = 11.6 Hz, 2H), 2.93-2.84 (m, 1H), 2.75-2.69 |
| | | | | (m, 1H), 2.65-2.59 (m, 2H), 2.23-2.17 (m, 4H), |
| | | | | 2.11-2.04 (dt, J = 2.8, 11.6 Hz, 2H), 2.02-1.95 |
| | | | | (m, 5H), 1.85-1.75 (m, 4H), 1.71-1.65 (m, 1H), |
| | | | | 1.23-1.10 (m, 2H) |
| I-79 | BSA | BRR | 755.2 | 10.52 (s, 1H), 10.20 (s, 1H), 9.43 (s, 1H), 9.07 (s, |
| | | | | 1H), 8.62 (s, 1H), 8.53 (s, 1H), 8.50-8.47 (m, |
| | | | | 2H), 8.42 (t, J = 7.6 Hz, 1H), 8.23 (d, J = 7.6 Hz, |
| | | | | 1H), 7.75-7.68 (m, 1H), 7.60 (d, J = 8.4 Hz, 1H), |
| | | | | 7.26 (d, J = 7.6 Hz, 1H), 4.64-4.53 (m, 1H), 3.95- |
| | | | | 3.85 (m, 1H), 3.75-3.65 (m, 1H), 3.52-3.45 |
| | | | | (m, 2H), 3.00-2.92 (m, 1H), 2.91-2.79 (m, 2H), |
| | | | | 2.79-2.71 (m, 1H), 2.61-2.54 (m, 1H), 2.37 (d, |
| | | | | J = 6.8 Hz, 2H), 2.32 (s, 3H), 2.22-2.19 (m, 2H), |
| | | | | 2.06-1.94 (m, 4H), 1.88 (m, 4H), 1.68-1.55 (m, |
| | | | | 1H), 1.23-1.09 (m, 2H) |
| I-82 | BSP | BRR | 762.4 | 11.11 (s, 1H), 10.20 (s, 1H), 9.06 (s, 1H), 8.61 (s, |
| | | | | 1H), 8.50-8.47 (m, 2H), 8.42 (t, J = 7.6 Hz, 1H), |
| | | | | 8.23 (d, J = 8.0 Hz, 1H), 7.08-6.98 (m, 1H), 6.92- |
| | | | | 6.85 (m, 1H), 5.40-5.35 (m, 1H), 4.63-4.55 |
| | | | | (m, 1H), 3.60 (s, 2H), 3.37-3.35 (m, 1H), 3.30 (s, |
| | | | | 3H), 3.19-3.02 (m, 2H), 2.92-2.83 (m, 1H), 2.75- |
| | | | | 2.69 (m, 1H), 2.65-2.52 (m, 3H), 2.23-2.20(m, 3H), |
| | | | | 2.02-1.94 (m, 6H), 1.86-1.71 (m, 3H), 1.24-1.14 (m, 2H) |
| I-83 | BRE | BRR | 774.5 | 11.08 (s, 1H), 10.21 (s, 1H), 9.07 (s, 1H), 8.62 (s, |
| | | | | 1H), 8.51-8.48 (m, 2H), 8.42 (t, J = 8.0 Hz, 1H), |
| | | | | 8.24 (dd, J = 0.8, 7.6 Hz, 1H), 6.91 (d, J = 8.8 Hz, |
| | | | | 1H), 6.73 (d, J = 8.8 Hz, 1H), 5.33 (dd, J = 5.6, |
| | | | | 12.4 Hz, 1H), 4.64-4.54 (m, 1H), 3.77 (s, 3H), |
| | | | | 3.57 (s, 3H), 2.99 (d, J = 10.4 Hz, 2H), 2.92-2.84 |
| | | | | (m, 1H), 2.71-2.60 (m, 2H), 2.42 (d, J = 11.6 Hz, |
| | | | | 2H), 2.22 (d, J = 6.8 Hz, 4H), 2.07-1.94 (m, 8H), |
| | | | | 1.76-1.64 (m, 1H), 1.59 (d, J = 12.0 Hz, 2H), 1.22- |
| | | | | 1.12 (m, 2H) |
| I-84 | AQK | BSU | 800.5 | 11.08 (s, 1H), 8.81 (d, J = 2.0 Hz, 1H), 8.67 (d, J = |
| | | | | 2.0 Hz, 1H), 8.62 (d, J = 7.2 Hz, 1H), 8.59-8.54 |
| | | | | (m, 2H), 8.52 (d, J = 3.9 Hz, 1H), 8.08 (s, 1H), |
| | | | | 6.99-6.93 (m, 1H), 6.92-6.84 (m, 3H), 5.34 (dd, |
| | | | | J = 5.2, 12.8 Hz, 1H), 3.84-3.74 (m, 1H), 3.62 (s, |
| | | | | 3H), 3.17-3.09 (m, 4H), 2.93-2.83 (m, 1H), 2.78- |
| | | | | 2.54 (m, 6H), 2.36-2.25 (m, 4H), 2.04-1.95 (m, 1H), |
| | | | | 1.81 (t, J = 12.4 Hz, 6H), 1.72-1.61 (m, 2H), 1.56- |
| | | | | 1.41 (m, 2H), 1.29 (d, J = 6.4 Hz, 6H), 1.01-0.82 (m, 4H) |
| I-85 | AZK | BSU | 771.5 | 11.08 (s, 1H), 8.81 (d, J = 2.0 Hz, 1H), 8.67 (d, J = |
| | | | | 2.0 Hz, 1H), 8.63 (d, J = 7.2 Hz, 1H), 8.59-8.55 |
| | | | | (m, 2H), 8.52 (d, J = 4.0 Hz, 1H), 8.08 (s, 1H), |
| | | | | 7.06-6.93 (m, 3H), 6.88 (d, J = 4.0 Hz, 1H), 5.40- |
| | | | | 5.32 (m, 1H), 3.83-3.72 (m, 1H), 3.57 (s, 3H), |
| | | | | 3.23-3.18 (m, 1H), 3.14-3.06 (m, 2H), 3.01- |
| | | | | 2.92 (m, 2H), 2.90-2.82 (m, 1H), 2.74-2.58 (m, |
| | | | | 2H), 2.16 (d, J = 5.6 Hz, 2H), 2.09-1.97 (m, 3H), |
| | | | | 1.85-1.73 (m, 8H), 1.55-1.45 (m, 2H), 1.29 (d, |
| | | | | J = 6.4 Hz, 6H), 0.99-0.80 (m, 4H) |
| I-88 | BQL | BRR | 778.4 | 11.12 (s, 1H), 10.20 (s, 1H), 9.07 (s, 1H), 8.61 (s, |
| | | | | 1H), 8.51-8.47 (m, 2H), 8.44-8.39 (m, 1H), 8.24 |
| | | | | (d, J = 8.4 Hz, 1H), 7.14-7.04 (m, 2H), 5.40 (dd, |
| | | | | J = 5.2, 12.5 Hz, 1H), 4.65-4.57 (m, 1H), 3.66 (s, |
| | | | | 3H), 3.10-2.97 (m, 2H), 2.93-2.83 (m, 2H), 2.75- |
| | | | | 2.70 (m, 1H), 2.68-2.63 (m, 2H), 2.62-2.57 |
| | | | | (m, 1H), 2.26-2.17 (m, 3H), 2.07-1.91 (m, 8H), |
| | | | | 1.78-1.58 (m, 2H), 1.34-1.13 (m, 3H) |
| I-96 | BSA | ATJ | 784.3 | 10.52 (d, J = 9.2 Hz, 2H), 9.43 (s, 1H), 8.69 (s, |
| | | | | 1H), 8.53 (s, 1H), 8.49-8.44 (m, 1H), 8.44-8.38 |
| | | | | (m, 1H), 8.35 (s, 1H), 8.22 (dd, J = 1.2, 7.6 Hz, |
| | | | | 1H), 7.74-7.67 (m, 1H), 7.60 (d, J = 8.4 Hz, 1H), |
| | | | | 7.26 (d, J = 7.6 Hz, 1H), 7.16 (s, 1H), 4.44-4.34 |
| | | | | (m, 1H), 3.98 (s, 3H), 3.90 (ddd, J = 5.2, 10.0, 12.2 |
| | | | | Hz, 1H), 3.70 (td, J = 6.0, 12.2 Hz, 1H), 3.50- |
| | | | | 3.42 (m, 2H), 2.96 (ddd, J = 6.0, 10.0, 16.4 Hz, |
| | | | | 1H), 2.90-2.81 (m, 2H), 2.80-2.71 (m, 1H), 2.64- |
| | | | | 2.54 (m, 1H), 2.39 (d, J = 6.4 Hz, 2H), 2.33 (s, |
| | | | | 3H), 2.21-2.10 (m, 2H), 2.00 (d, J = 11.6 Hz, 2H), |
| | | | | 1.85 (s, 6H), 1.67-1.53 (m, 1H), 1.21-1.08 (m, 2H) |
| I-97 | BSN | ATJ | 754.2 | 10.54 (s, 1H), 10.51 (s, 1H), 9.66 (s, 1H), 8.70 (s, |
| | | | | 1H), 8.59 (s, 1H), 8.46 (d, J = 8.0 Hz, 1H), 8.43- |
| | | | | 8.38 (m, 1H), 8.35 (s, 1H), 8.22 (d, J = 7.6 Hz, |
| | | | | 1H), 7.91-7.87 (d, J = 8.4 Hz, 1H), 7.84-7.78 |
| | | | | (m, 1H), 7.67-7.62 (m, 1H), 7.16 (s, 1H), 4.46- |
| | | | | 4.36 (m, 1H), 3.99 (s, 3H), 3.96-3.88 (m, 1H), |
| | | | | 3.85-3.65 (m, 2H), 3.04-2.93 (m, 1H), 2.91-2.56 |
| | | | | (m, 4H), 2.54-2.51 (m, 3H), 2.23-2.14 (m, 2H), 2.11- |
| | | | | 1.89 (m, 8H), 1.87-1.75 (m, 1H), 1.30-1.13 (m, 2H) |
| I-98 | BSA | BTW | 754.2 | 10.52 (s, 1H), 10.36 (s, 1H), 9.43 (s, 1H), 8.53 (s, |
| | | | | 1H), 8.44-8.37 (m, 2H), 8.37-8.21 (m, 2H), 8.17 |
| | | | | (d, J = 7.5 Hz, 1H), 7.74-7.68 (m, 1H), 7.64- |
| | | | | 7.58 (m, 2H), 7.57-7.53 (m, 1H), 7.26 (d, J = 7.5 |
| | | | | Hz, 1H), 4.52-4.40 (m, 1H), 3.90 (d, J = 5.1, 9.8, |
| | | | | 12.2 Hz, 1H), 3.74-3.66 (m, 1H), 3.55-3.40 (m, |
| | | | | 3H), 3.04-2.91 (m, 1H), 2.90-2.71 (m, 3H), 2.36 |
| | | | | (br d, J = 6.5 Hz, 2H), 2.32 (s, 3H), 2.18 (br d, J = |
| | | | | 10.8 Hz, 2H), 2.09-1.92 (m, 4H), 1.88 (s, 4H), |
| | | | | 1.67-1.52 (m, 1H), 1.22-1.08 (m, 2H) |
| I-99 | BQM | BTW | 757.3 | 11.08 (s, 1H), 10.36 (s, 1H), 8.42-8.33 (m, 3H), |
| | | | | 8.30 (s, 1H), 8.17 (d, J = 7.6 Hz, 1H), 7.63 (d, J = |
| | | | | 9.2 Hz, 1H), 7.57-7.53 (m, 1H), 6.92-6.87 (m, |
| | | | | 1H), 6.85-6.81 (m, 1H), 5.38-5.31 (m, 1H), 4.52- |
| | | | | 4.43 (m, 1H), 3.63 (s, 3H), 3.61-3.56 (m, 1H), |
| | | | | 3.25-3.23 (m, 1H), 2.94-2.83 (m, 1H), 2.76- |
| | | | | 2.58 (m, 3H), 2.54-2.51 (m, 2H), 2.47 (s, 3H), |
| | | | | 2.40-2.29 (m, 3H), 2.23-2.15 (m, 2H), 2.05- |
| | | | | 1.90 (m, 6H), 1.86-1.73 (m, 3H), 1.27-1.74 (m, 2H) |
| I-100 | AQK | BUA | 826.4 | 11.10 (s, 1H), 8.93 (s, 1H), 8.51 (s, 1H), 8.41 (d, J = |
| | | | | 8.0 Hz, 1H), 8.28-8.22 (m, 1H), 8.02 (s, 1H), |
| | | | | 7.90 (d, J = 8.0 Hz, 1H), 7.28 (s, 1H), 7.02-6.86 |
| | | | | (m, 3H), 5.36 (dd, J = 5.6, 12.8 Hz, 1H), 4.53- |
| | | | | 4.43 (m, 1H), 3.87 (s, 3H), 3.65 (s, 3H), 3.16 (d, J = |
| | | | | 10.8 Hz, 2H), 2.96-2.85 (m, 1H), 2.77-2.61 |
| | | | | (m, 4H), 2.48-2.42 (m, 1H), 2.31 (d, J = 6.4 Hz, |
| | | | | 2H), 2.27 (s, 3H), 2.18 (d, J = 10.8 Hz, 2H), 2.03- |
| | | | | 1.89 (m, 5H), 1.81 (d, J = 10.8 Hz, 2H), 1.73- |
| | | | | 1.53 (m, 3H), 1.20-1.07 (m, 2H) |
| I-101 | BSP | ATJ | 791.3 | 11.11 (s, 1H), 10.51 (s, 1H), 8.71-8.67 (m, 1H), |
| | | | | 8.47 (d, J = 1.2, 7.2 Hz, 1H), 8.43-8.38 (m, 1H), |
| | | | | 8.34 (s, 1H), 8.24-8.20 (m, 1H), 7.18-7.14 (m, |
| | | | | 1H), 7.09-6.99 (m, 1H), 6.94-6.86 (m, 1H), 5.43- |
| | | | | 5.36 (m, 1H), 4.46-4.36 (m, 1H), 3.99 (s, 3H), |
| | | | | 3.63 (s, 2H), 3.36 (s, 1H), 3.30 (s, 3H), 2.92-2.83 |
| | | | | (m, 1H), 2.76-2.58 (m, 3H), 2.53-2.52 (m, 3H), |
| | | | | 2.32-2.25 (m, 1H), 2.24-2.11 (m, 3H), 2.05- |
| | | | | 1.83 (m, 8H), 1.31-1.14 (m, 2H) |
| I-102 | BQM | BRR | 758.4 | 11.08 (s, 1H), 10.20 (s, 1H), 9.07 (s, 1H), 8.61 (s, |
| | | | | 1H), 8.52-8.46 (m, 2H), 8.41 (t, J = 7.6 Hz, 1H), |
| | | | | 8.23 (d, J = 7.6 Hz, 1H), 6.93-6.78 (m, 2H), 5.34 |
| | | | | (dd, J = 5.2, 12.6 Hz, 1H), 4.66-4.54 (m, 1H), |
| | | | | 3.63 (s, 3H), 3.61-3.53 (m, 1H), 3.24-3.16 (m, |
| | | | | 2H), 2.95-2.83 (m, 1H), 2.75-2.55 (m, 3H), 2.47 |
| | | | | (s, 3H), 2.39-2.27 (m, 3H), 2.22 (d, J = 9.6 Hz, |
| | | | | 2H), 2.07-1.91 (m, 6H), 1.77 (d, J = 9.6 Hz, 4H), |
| | | | | 1.30-1.13 (m, 2H) |
| I-103 | BSN | BRR | 726.6 | 10.54 (s, 1H), 10.20 (s, 1H), 9.64 (s, 1H), 9.07 (d, |
| | | | | J = 1.0 Hz, 1H), 8.60 (d, J = 14.2 Hz, 2H), 8.52- |
| | | | | 8.45 (m, 2H), 8.41 (t, J = 7.8 Hz, 1H), 8.23 (dd, J = |
| | | | | 1.0, 7.8 Hz, 1H), 7.91-7.84 (m, 1H), 7.80 (t, J = |
| | | | | 7.8 Hz, 1H), 7.65 (d, J = 6.6 Hz, 1H), 4.66-4.54 |
| | | | | (m, 1H), 3.93 (ddd, J = 5.2, 9.8, 12.2 Hz, 1H), 3.72 |
| | | | | (d, J = 6.0, 12.2 Hz, 2H), 3.25-3.06 (m, 2H), 3.02- |
| | | | | 2.93 (m, 1H), 2.80-2.73 (m, 1H), 2.64-2.52 |
| | | | | (m, 1H), 2.39 (dd, J = 3.0, 8.8 Hz, 2H), 2.22 (d, J = |
| | | | | 10.4 Hz, 2H), 2.10-1.90 (m, 8H), 1.86-1.75 |
| | | | | (m, 1H), 1.29-1.15 (m, 2H) |
| I-104 | BUC | BRR | 774.6 | 11.08 (s, 1H), 10.20 (s, 1H), 9.07 (s, 1H), 8.62 (s, |
| | | | | 1H), 8.51-8.47 (m, 2H), 8.45-8.39 (m, 1H), 8.24 |
| | | | | (d, J = 7.6 Hz, 1H), 6.93 (d, J = 8.4 Hz, 1H), 6.73 |
| | | | | (d, J = 8.8 Hz, 1H), 5.39-5.27 (m, 1H), 4.60 (t, J = |
| | | | | 11.6 Hz, 1H), 3.78 (s, 3H), 3.58 (s, 3H), 3.40 (s, |
| | | | | 1H), 3.18 (s, 2H), 2.96-2.83 (m, 1H), 2.76-2.58 |
| | | | | (m, 3H), 2.49-2.42 (m, 4H), 2.22 (d, J = 8.4 Hz, |
| | | | | 2H), 2.10-1.89 (m, 6H), 1.86-1.74 (m, 1H), 1.67 |
| | | | | (d, J = 10.4 Hz, 2H), 1.31-1.12 (m, 2H) |
| I-105^{b} | AQK | BVL | 800.5 | 11.08 (s, 1H), 8.81 (d, J = 2.0 Hz, 1H), 8.67 (d, J = |
| | | | | 2.0 Hz, 1H), 8.62 (d, J = 7.2 Hz, 1H), 8.59-8.54 |
| | | | | (m, 2H), 8.52 (d, J = 3.9 Hz, 1H), 8.08 (s, 1H), |
| | | | | 6.99-6.93 (m, 1H), 6.92-6.84 (m, 3H), 5.34 (dd, |
| | | | | J = 5.2, 12.8 Hz, 1H), 3.84-3.74 (m, 1H), 3.62 (s, |
| | | | | 3H), 3.17-3.09 (m, 4H), 2.93-2.83 (m, 1H), 2.78- |
| | | | | 2.54 (m, 6H), 2.36-2.25 (m, 4H), 2.04-1.95 |
| | | | | (m, 1H), 1.81 (t, J = 12.4 Hz, 6H), 1.72-1.61 (m, |
| | | | | 2H), 1.56-1.41 (m, 2H), 1.29 (d, J = 6.4 Hz, 6H), |
| | | | | 1.01-0.82 (m, 4H) |
| I-106 | BQL | BTW | 777.4 | 11.16-11.06 (m, 1H), 10.36 (s, 1H), 8.42-8.33 |
| | | | | (m, 3H), 8.30 (s, 1H), 8.21-8.15 (m, 1H), 7.66- |
| | | | | 7.52 (m, 2H), 7.14-7.00 (m, 2H), 5.39 (dd, J = |
| | | | | 5.2, 12.8 Hz, 1H), 4.50-4.40 (m, 1H), 3.62 (s, |
| | | | | 3H), 3.54-3.46 (m, 1H), 3.01 (d, J = 10.4 Hz, |
| | | | | 2H), 2.92-2.82 (m, 1H), 2.75-2.67 (m, 1H), 2.67 |
| | | | | (s, 3H), 2.24-2.14 (m, 4H), 2.07-1.95 (m, 6H), |
| | | | | 1.93 (s, 1H), 1.65 (d, J = 11.2 Hz, 3H), 1.22-1.07 |
| | | | | (m, 2H) |
| I-107 | AZK | BUE | 786.7 | 11.09 (s, 1H), 11.05 (s, 1H), 8.71 (s, 1H), 8.51- |
| | | | | 8.44 (m, 1H), 8.43-8.37 (m, 1H), 8.35 (s, 1H), |
| | | | | 8.21 (d, J = 8.0 Hz, 1H), 7.44 (s, 1H), 7.06-6.93 |
| | | | | (m, 3H), 5.37 (dd, J = 5.2, 12.4 Hz, 1H), 4.47- |
| | | | | 4.33 (m, 1H), 3.59 (s, 3H), 3.21-3.16 (m, 1H), |
| | | | | 2.99 (d, J = 11.2 Hz, 2H), 2.94-2.83 (m, 1H), 2.74 |
| | | | | (s, 6H), 2.72-2.60 (m, 2H), 2.21 (d, J = 7.2 Hz, |
| | | | | 2H), 2.18-2.12 (m, 2H), 2.11-2.03 (m, 2H), 2.02- |
| | | | | 1.86 (m, 5H), 1.84-1.71 (m, 4H), 1.70-1.59 |
| | | | | (m, 1H), 1.20-1.05 (m, 2H) |
| I-108 | BQM | BUL | 787.6 | 11.07 (s, 1H), 10.01 (s, 1H), 9.37 (s, 1H), 9.18 (s, |
| | | | | 1H), 8.67 (s, 1H), 8.32 (s, 1H), 8.03 (s, 1H), 7.09 |
| | | | | (s, 1H), 6.90-6.79 (m, 2H), 5.34 (dd, J = 5.2, 12.8 |
| | | | | Hz, 1H), 4.47-4.31 (m, 1H), 3.87 (s, 3H), 3.61 (s, |
| | | | | 3H), 3.50-3.42 (m, 1H), 3.01-2.99 (m, 2H), 2.93- |
| | | | | 2.82 (m, 1H), 2.70-2.56 (m, 2H), 2.46 (s, 3H), |
| | | | | 2.30-2.13 (m, 6H), 2.08-1.85 (m, 7H), 1.96- |
| | | | | 1.69 (m, 3H), 1.21-1.07 (m, 2H) |
| I-109^{b} | BQM | BVB | 744.6 | 11.07 (s, 1H), 9.90 (s, 1H), 9.31 (s, 1H), 9.21 (d, J = |
| | | | | 1.6 Hz, 1H), 8.78 (s, 1H), 8.31 (s, 1H), 8.08 (s, |
| | | | | 1H), 7.09 (s, 1H), 6.90-6.85 (m, 1H), 6.84-6.80 |
| | | | | (m, 1H), 5.34 (dd, J = 5.2, 12.4 Hz, 1H), 4.43- |
| | | | | 4.34 (m, 1H), 3.88 (s, 3H), 3.61 (s, 3H), 3.49- |
| | | | | 3.42 (m, 1H), 3.02 (d, J = 10.4 Hz, 2H), 2.92-2.83 |
| | | | | (m, 1H), 2.67 (d, J = 3.6 Hz, 1H), 2.64-2.58 (m, |
| | | | | 1H), 2.46 (s, 2H), 2.31-2.12 (m, 7H), 2.08-1.86 |
| | | | | (m, 8H), 1.69 (d, J = 12.0 Hz, 3H), 1.18-1.10 (m, 2H) |
| I-110 | BQM | BUK | 753.5 | 11.07 (s, 1H), 9.82 (s, 1H), 9.05 (s, 1H), 8.83 (d, J = |
| | | | | 2.0 Hz, 1H), 8.41 (s, 1H), 8.31 (s, 1H), 8.03 (s, |
| | | | | 1H), 7.08 (s, 1H), 6.94-6.75 (m, 2H), 5.40-5.23 |
| | | | | (m, 1H), 4.46-4.29 (m, 1H), 3.87 (s, 3H), 3.61 (s, |
| | | | | 3H), 3.51-3.39 (m, 1H), 3.05-2.96 (m, 2H), 2.91- |
| | | | | 2.81 (m, 1H), 2.71-2.61 (m, 2H), 2.46 (s, 3H), |
| | | | | 2.29-2.13 (m, 6H), 2.06-1.83 (m, 7H), 1.74- |
| | | | | 1.60 (m, 3H), 1.20-1.08 (m, 2H) |
| I-111 | BUC | BUK | 769.6 | 11.07 (s, 1H), 9.83 (s, 1H), 9.05 (s, 1H), 8.83 (d, J = |
| | | | | 2.3 Hz, 1H), 8.41 (s, 1H), 8.31 (s, 1H), 8.03 (s, |
| | | | | 1H), 7.08 (s, 1H), 6.91 (d, J = 8.4 Hz, 1H), 6.72 |
| | | | | (d, J = 8.4 Hz, 1H), 5.39-5.27 (m, 1H), 4.47- |
| | | | | 4.31 (m, 1H), 3.87 (s, 3H), 3.77 (s, 3H), 3.56 (s, |
| | | | | 3H), 2.97 (d, J = 10.0 Hz, 2H), 2.89-2.82 (m, |
| | | | | 1H), 2.71-2.62 (m, 2H), 2.42-2.32 (m, 3H), 2.21- |
| | | | | 2.11 (m, 4H), 2.04-1.88 (m, 7H), 1.58 (d, J = |
| | | | | 11.2 Hz, 3H), 1.20-1.09 (m, 2H) |
| I-112 | AZK | BUM | 744.2 | 12.34 (s, 1H), 11.09 (s, 1H), 8.55 (dd, J = 1.6, 4.8 |
| | | | | Hz, 1H), 8.51 (s, 1H), 8.31 (s, 1H), 8.22 (dd, J = |
| | | | | 1.6, 8.0 Hz, 1H), 8.09 (d, J = 4.0 Hz, 1H), 7.44- |
| | | | | 7.38 (m, 1H), 7.15 (s, 1H), 7.05-6.92 (m, 3H), |
| | | | | 6.81 (d, J = 4.0 Hz, 1H), 5.37 (dd, J = 5.6, 12.4 |
| | | | | Hz, 1H), 4.41-4.32 (m, 1H), 4.05 (s, 3H), 3.59 (s, |
| | | | | 3H), 3.22-3.17 (m, 1H), 2.99 (d, J = 11.2 Hz, 2H), |
| | | | | 2.94-2.84 (m, 1H), 2.78-2.68 (m, 1H), 2.66- |
| | | | | 2.58 (m, 1H), 2.21 (d, J = 7.2 Hz, 2H), 2.15 (d, J = |
| | | | | 12.0 Hz, 2H), 2.10-2.04 (m, 2H), 2.01-1.95 |
| | | | | (m, 3H), 1.93-1.85 (m, 2H), 1.83-1.75 (m, 4H), |
| | | | | 1.70-1.60 (m, 1H), 1.18-1.07 (m, 2H) |
| I-113 | AQK | BUM | 773.3 | 12.34 (s, 1H), 11.09 (s, 1H), 8.55 (dd, J = 1.6, 4.8 |
| | | | | Hz, 1H), 8.51 (s, 1H), 8.31 (s, 1H), 8.22 (dd, J = |
| | | | | 1.6, 7.6 Hz, 1H), 8.09 (d, J = 4.0 Hz, 1H), 7.44- |
| | | | | 7.38 (m, 1H), 7.14 (s, 1H), 7.00-6.94 (m, 1H), |
| | | | | 6.93-6.84 (m, 2H), 6.81 (d, J = 4.0 Hz, 1H), 5.35 |
| | | | | (dd, J = 5.2, 12.4 Hz, 1H), 4.44-4.30 (m, 1H), |
| | | | | 4.05 (s, 3H), 3.64 (s, 3H), 3.15 (d, J = 10.8 Hz, |
| | | | | 2H), 2.95-2.84 (m, 1H), 2.76-2.58 (m, 4H), 2.46- |
| | | | | 2.42 (m, 1H), 2.30 (d, J = 6.8 Hz, 2H), 2.26 (s, |
| | | | | 3H), 2.21-2.10 (m, 2H), 2.03-1.84 (m, 5H), 1.84- |
| | | | | 1.75 (m, 2H), 1.73-1.60 (m, 2H), 1.59-1.48 |
| | | | | (m, 1H), 1.19-1.03 (m, 2H) |
| I-114 | AZK | BUR | 757.6 | 11.10 (s, 1H), 8.82 (d, J = 2.0 Hz, 1H), 8.68 (d, J = |
| | | | | 2.0 Hz, 1H), 8.63-8.56 (m, 2H), 8.53 (d, J = 4.0 |
| | | | | Hz, 1H), 8.34 (d, J = 8.0 Hz, 1H), 8.09 (s, 1H), |
| | | | | 7.05-6.95 (m, 3H), 6.90 (d, J = 4.0 Hz, 1H), 5.38 |
| | | | | (dd, J = 5.2, 12.8 Hz, 1H), 3.82-3.71 (m, 2H), |
| | | | | 3.59 (s, 3H), 3.25-3.18 (m, 1H), 3.02-2.85 (m, |
| | | | | 3H), 2.78-2.59 (m, 2H), 2.18 (d, J = 6.4 Hz, 2H), |
| | | | | 2.10-1.97 (m, 3H), 1.93-1.75 (m, 8H), 1.57- |
| | | | | 1.46 (m, 1H), 1.42-1.33 (m, 2H), 1.30 (d, J = 6.4 |
| | | | | Hz, 6H), 1.04-0.91 (m, 2H) |
| I-115 | AQK | BUS | 772.6 | 11.08 (s, 1H), 8.80 (d, J = 2.0 Hz, 1H), 8.67 (d, J = |
| | | | | 2.0 Hz, 1H), 8.49 (d, J = 4.0 Hz, 1H), 8.03 (d, J = |
| | | | | 5.6 Hz, 2H), 7.00-6.94 (m, 1H), 6.92-6.84 (m, |
| | | | | 3H), 6.22 (d, J = 7.6 Hz, 1H), 5.35 (dd, J = 5.2, |
| | | | | 12.4 Hz, 1H), 4.33-3.87 (m, 2H), 3.84-3.74 (m, |
| | | | | 1H), 3.63 (s, 3H), 3.15 (d, J = 10.8 Hz, 2H), 3.01- |
| | | | | 2.84 (m, 3H), 2.77-2.56 (m, 5H), 2.42-2.28 |
| | | | | (m, 6H), 2.04-1.95 (m, 1H), 1.79 (d, J = 8.8 Hz, |
| | | | | 4H), 1.66 (d, J = 9.6 Hz, 2H), 1.28 (d, J = 6.4 Hz, |
| | | | | 6H), 1.19-1.09 (m, 2H) |
| I-116 | BUC | BUT | 739.2 | 11.07 (s, 1H), 10.45 (s, 1H), 9.06 (d, J = 1.6 Hz, |
| | | | | 1H), 8.83 (d, J = 2.4 Hz, 1H), 8.44 (t, J = 2.0 Hz, |
| | | | | 1H), 8.40 (s, 1H), 8.25 (s, 1H), 7.61 (d, J = 9.2 Hz, |
| | | | | 1H), 7.45 (dd, J = 1.6, 9.2 Hz, 1H), 6.91 (d, J = |
| | | | | 8.4 Hz, 1H), 6.72 (d, J = 8.8 Hz, 1H), 5.32 (dd, J = |
| | | | | 5.2, 12.8 Hz, 1H), 4.52-4.36 (m, 1H), 3.77 (s, 3H), |
| | | | | 3.56 (s, 3H), 2.97 (d, J = 10.0 Hz, 2H), 2.93- |
| | | | | 2.83 (m, 1H), 2.76-2.61 (m, 2H), 2.40 (s, 2H), |
| | | | | 2.23-2.14 (m, 4H), 2.05-1.79 (m, 8H), 1.74-1.63 (m, |
| | | | | 1H), 1.58 (d, J = 12.4 Hz, 2H), 1.21-1.07 (m, 2H) |
| I-117 | BSA | BUK | 750.5 | 10.53 (s, 1H), 9.83 (s, 1H), 9.43 (s, 1H), 9.05 (s, |
| | | | | 1H), 8.83 (d, J = 2.0 Hz, 1H), 8.53 (s, 1H), 8.41 |
| | | | | (s, 1H), 8.31 (s, 1H), 8.03 (s, 1H), 7.76-7.67 (m, |
| | | | | 1H), 7.60 (d, J = 8.4 Hz, 1H), 7.26 (d, J = 7.6 Hz, |
| | | | | 1H), 7.08 (s, 1H), 4.48-4.31 (m, 1H), 3.95-3.89 |
| | | | | (m, 1H), 3.87 (s, 3H), 3.73-3.67 (m, 1H), 3.53- |
| | | | | 3.43 (m, 3H), 3.00-2.92 (m, 1H), 2.92-2.80 (m, |
| | | | | 2H), 2.80-2.71 (m, 1H), 2.62-2.53 (m, 1H), 2.37 |
| | | | | (d, J = 6.8 Hz, 2H), 2.32 (s, 3H), 2.16 (d, J = 10.8 |
| | | | | Hz, 2H), 2.00 (d, J = 11.6 Hz, 2H), 1.94-1.82 (m, |
| | | | | 5H), 1.65-1.53 (m, 1H), 1.18-1.09 (m, 2H) |
| I-118 | AQK | BUR | 786.4 | 11.09 (s, 1H), 8.81 (d, J = 2.0 Hz, 1H), 8.67 (d, J = |
| | | | | 2.0 Hz, 1H), 8.62-8.55 (m, 2H), 8.52 (d, J = 4.0 |
| | | | | Hz, 1H), 8.32 (d, J = 7.6 Hz, 1H), 8.08 (s, 1H), |
| | | | | 7.01-6.93 (m, 1H), 6.93-6.84 (m, 3H), 5.35 (dd, |
| | | | | J = 5.6, 12.4 Hz, 1H), 3.75 (d, J = 6.0, 12.8 Hz, |
| | | | | 2H), 3.64 (s, 3H), 3.15 (d, J = 11.2 Hz, 2H), 2.95- |
| | | | | 2.84 (m, 1H), 2.77-2.58 (m, 4H), 2.45-2.39 (m, |
| | | | | 1H), 2.28-2.21 (m, 5H), 2.04-1.96 (m, 1H), 1.92-1.75 |
| | | | | (m, 6H), 1.71-1.57 (m, 2H), 1.44-1.31 (m, 3H), 1.29 |
| | | | | (d, J = 6.4 Hz, 6H), 0.94 (q, J = 12.0 Hz, 2H) |
| I-119 | BRY | BUY | 727.2 | 10.52 (d, J = 6.4 Hz, 2H), 9.25 (s, 1H), 8.69 (s, |
| | | | | 1H), 8.50-8.40 (m, 3H), 8.38 (s, 1H), 8.23-8.20 |
| | | | | (m, 1H), 8.06 (s, 1H), 7.91 (d, J = 8.4 Hz, 1H), |
| | | | | 7.79 (dd, J = 1.6, 8.8 Hz, 1H), 7.20 (s, 1H), 5.27- |
| | | | | 5.17 (m, 1H), 3.99 (s, 3H), 3.96-3.89 (m, 1H), |
| | | | | 3.72 (td, J = 6.0, 12.0 Hz, 1H), 3.06 (d, J = 11.6 |
| | | | | Hz, 2H), 3.00-2.92 (m, 1H), 2.79-2.70 (m, 5H), |
| | | | | 2.62-2.59 (s, 2H), 2.37-2.32 (m, 2H), 2.18-2.12 |
| | | | | (m, 2H), 1.89-1.76 (m, 4H) |
| I-120 | BRY | BTO | 772.6 | 10.53 (s, 1H), 9.88 (s, 1H), 9.26 (s, 1H), 8.48 (s, |
| | | | | 1H), 8.31 (s, 1H), 8.17 (d, J = 3.2 Hz, 1H), 8.11 |
| | | | | (d, J = 8.4 Hz, 1H), 8.07 (s, 1H), 7.99-7.94 (m, |
| | | | | 2H), 7.92 (d, J = 8.8 Hz, 1H), 7.79 (dd, J = 1.4, |
| | | | | 8.8 Hz, 1H), 7.08 (s, 1H), 4.44-4.35 (m, 1H), 3.93 |
| | | | | (m, 1H), 3.86 (s, 3H), 3.76-3.69 (m, 1H), 3.06- |
| | | | | 2.99 (m, 2H), 2.99-2.91 (m, 1H), 2.76 (td, J = |
| | | | | 5.6, 16.8 Hz, 2H), 2.23 (d, J = 7.0 Hz, 2H), 2.17 |
| | | | | (d, J = 11.2 Hz, 2H), 2.07 (t, J = 10.4 Hz, 2H), |
| | | | | 1.99 (d, J = 15.0 Hz, 2H), 1.93 (d, J = 2.4 Hz, 1H), |
| | | | | 1.91-1.84 (m, 3H), 1.80 (d, J = 10.0 Hz, 2H), 1.73- |
| | | | | 1.62 (m, 1H), 1.21-1.09 (m, 2H) |
| I-121 | BSN | BVB | 712.3 | 10.54 (s, 1H), 9.90 (s, 1H), 9.61 (s, 1H), 9.31 (s, |
| | | | | 1H), 9.21 (d, J = 1.6 Hz, 1H), 8.78 (s, 1H), 8.57 |
| | | | | (s, 1H), 8.32 (s, 1H), 8.08 (s, 1H), 7.89-7.83 (m, |
| | | | | 1H), 7.78 (t, J = 7.6 Hz, 1H), 7.66 (d, J = 7.2 Hz, |
| | | | | 1H), 7.09 (s, 1H), 4.45-4.34 (m, 1H), 3.96-3.91 |
| | | | | (m, 1H), 3.88 (s, 3H), 3.75-3.68 (m, 1H), 3.64 |
| | | | | 3.56 (m, 1H), 3.08-3.01 (m, 2H), 3.01-2.93 (m, |
| | | | | 1H), 2.80-2.72 (m, 1H), 2.60-2.55 (m, 2H), 2.28- |
| | | | | 2.26 (m, 2H), 2.25-2.21 (m, 1H), 2.20-2.13 (m, |
| | | | | 2H), 2.05-1.97 (m, 2H), 1.94-1.84 (m, 5H), 1.75- |
| | | | | 1.65 (m, 1H), 1.22-1.08 (m, 2H) |
| I-122 | BRY | BVB | 712.6 | 10.54 (s, 1H), 9.90 (s, 1H), 9.32-9.20 (m, 2H), |
| | | | | 8.78 (s, 1H), 8.48 (s, 1H), 8.31 (s, 1H), 8.14-8.06 |
| | | | | (m, 2H), 7.92 (d, J = 8.4 Hz, 1H), 7.78 (dd, J = |
| | | | | 1.6, 8.8 Hz, 1H), 7.63-7.45 (m, 1H), 7.08 (s, 1H), |
| | | | | 4.45-4.37 (m, 1H), 3.97-3.90 (m, 1H), 3.87 (s, |
| | | | | 3H), 3.72 (td, J = 6.0, 12.0 Hz, 1H), 3.17-3.07 |
| | | | | (m, 2H), 3.01-2.92 (m, 1H), 2.84-2.72 (m, 2H), |
| | | | | 2.53-2.51 (m, 2H), 2.38-2.34 (m, 1H), 2.19- |
| | | | | 2.15 (m, 2H), 2.03-1.96 (m, 2H), 1.94-1.82 (m, |
| | | | | 6H), 1.77-1.69 (m, 1H), 1.47-1.31 (m, 1H), 1.19- |
| | | | | 1.13 (m, 1H) |
| I-123 | BSN | BTO | 772.6 | 10.51 (s, 1H), 9.86 (s, 1H), 9.59 (s, 1H), 8.55 (s, |
| | | | | 1H), 8.29 (s, 1H), 8.22-8.14 (m, 1H), 8.12-8.05 |
| | | | | (m, 1H), 7.99-7.90 (m, 2H), 7.87-7.81 (m, 1H), |
| | | | | 7.76 (d, J = 7.6 Hz, 1H), 7.64 (d, J = 6.8 Hz, 1H), |
| | | | | 7.06 (s, 1H), 4.43-4.33 (m, 1H), 3.96-3.87 (m, |
| | | | | 1H), 3.84 (s, 3H), 3.74-3.65 (m, 1H), 3.62-3.53 |
| | | | | (m, 1H), 3.08-2.99 (m, 2H), 2.98-2.91 (m, 1H), |
| | | | | 2.81-2.69 (m, 1H), 2.31 (d, J = 1.6, 3.6 Hz, 1H), |
| | | | | 2.28-2.19 (m, 4H), 2.18-2.10 (m, 2H), 1.99 (d, |
| | | | | J = 1.6, 13.9 Hz, 2H), 1.93-1.83 (m, 5H), 1.72- |
| | | | | 1.60 (m, 1H), 1.19-1.06 (m, 2H) |
| I-124 | AZK | BVC | 769.3 | 12.42-12.25 (m, 1H), 11.09 (s, 1H), 8.59 (d, J = |
| | | | | 2.0 Hz, 1H), 8.55-8.48 (m, 1H), 8.30-8.22 (m, |
| | | | | 1H), 7.73-7.67 (m, 1H), 7.38 (s, 1H), 7.12 (s, |
| | | | | 1H), 7.06-6.94 (m, 3H), 6.61 (dd, J = 1.6, 3.6 Hz, |
| | | | | 1H), 5.37 (dd, J = 4.4, 12.4 Hz, 1H), 4.44-4.32 |
| | | | | (m, 1H), 3.95 (s, 3H), 3.59 (s, 3H), 3.26-3.18 (m, |
| | | | | 1H), 3.04-2.94 (m, 2H), 2.94-2.82 (m, 1H), 2.77- |
| | | | | 2.62 (m, 2H), 2.25-2.17 (m, 2H), 2.15-2.02 |
| | | | | (m, 4H), 2.01-1.85 (m, 5H), 1.83-1.73 (m, 4H), |
| | | | | 1.64 (m, 1H), 1.18-1.04 (m, 2H) |
| I-125 | BRY | BCN | 772.5 | 10.52 (d, J = 10.8 Hz, 2H), 9.25 (s, 1H), 9.01 (s, |
| | | | | 1H), 8.49-8.46 (m, 2H), 8.44-8.39 (m, 1H), 8.24 |
| | | | | (dd, J = 0.8, 7.6 Hz, 1H), 8.06 (s, 1H), 7.91 (d, J = |
| | | | | 8.8 Hz, 1H), 7.78 (dd, J = 1.6, 8.8 Hz, 1H), 7.71 |
| | | | | (s, 1H), 4.03 (s, 3H), 3.97-3.89 (m, 1H), 3.73 (td, |
| | | | | J = 5.6, 12.0 Hz, 1H), 3.11-3.05 (m, 1H), 3.04- |
| | | | | 3.00 (m, 2H), 2.96-2.91 (m, 1H), 2.79-2.72 (m, |
| | | | | 2H), 2.23-2.18 (m, 4H), 2.08-2.02 (m, 2H), 1.97 |
| | | | | (d, J = 10.8 Hz, 2H), 1.88-1.78 (m, 4H), 1.66- |
| | | | | 1.56 (m, 3H), 1.15-1.05 (m, 2H) |
| I-126 | AQK | BVC | 798.3 | 12.07 (s, 1H), 11.09 (s, 1H), 9.07 (d, J = 1.6 Hz, |
| | | | | 1H), 8.77 (d, J = 2.0 Hz, 1H), 8.50 (s, 1H), 8.33 |
| | | | | (s, 1H), 8.26 (d, J = 4.0 Hz, 1H), 7.15 (s, 1H), 7.00- |
| | | | | 6.94 (m, 1H), 6.93-6.84 (m, 3H), 5.35 (dd, J = |
| | | | | 5.2, 12.4 Hz, 1H), 4.46-4.31 (m, 1H), 4.08 (s, |
| | | | | 3H), 3.64 (s, 3H), 3.19-3.13 (m, 2H), 2.94-2.84 |
| | | | | (m, 1H), 2.78-2.69 (m, 2H), 2.66-2.57 (m, 1H), |
| | | | | 2.31-2.28 (m, 2H), 2.26 (s, 3H), 2.21-2.09 (m, |
| | | | | 3H), 2.04-1.85 (m, 6H), 1.84-1.77 (m, 2H), 1.73- |
| | | | | 1.61 (m, 2H), 1.60-1.50 (m, 1H), 1.17-1.05 (m, 2H) |
| I-127 | BRY | BUR | 739.7 | 10.54 (s, 1H), 9.27 (s, 1H), 8.82 (d, J = 2.0 Hz, |
| | | | | 1H), 8.68 (d, J = 2.0 Hz, 1H), 8.62-8.56 (m, 2H), |
| | | | | 8.53 (d, J = 4.0 Hz, 1H), 8.49 (s, 1H), 8.35 (d, J = |
| | | | | 7.6 Hz, 1H), 8.10-8.05 (m, 2H), 7.93 (d, J = 8.4 |
| | | | | Hz, 1H), 7.78 (dd, J = 1.4, 8.8 Hz, 1H), 6.89 (d, J = |
| | | | | 4.0 Hz, 1H), 3.94 (ddd, J = 5.1, 9.6, 12.2 Hz, |
| | | | | 1H), 3.81-3.70 (m, 3H), 3.16-3.07 (m, 2H), 2.97 |
| | | | | (ddd, J = 6.0, 9.2, 16.4 Hz, 1H), 2.84-2.72 (m, |
| | | | | 2H), 2.52 (d, J = 1.9 Hz, 2H), 2.28-2.17 (m, 2H), |
| | | | | 1.93-1.82 (m, 8H), 1.58 (dd, J = 6.0, 13.1 Hz, |
| | | | | 1H), 1.44-1.34 (m, 2H), 1.30 (d, J = 6.4 Hz, 6H), |
| | | | | 1.01 (q, J = 12.0 Hz, 2H) |
| I-128 | BVK | BVH | 718.6 | 11.10 (s, 1H), 10.59 (s, 1H), 8.43-8.38 (m, 2H), |
| | | | | 8.35-8.27 (m, 3H), 7.61 (s, 2H), 6.99 (dd, J = 4.0, |
| | | | | 8.8 Hz, 1H), 6.91-6.83 (m, 1H), 5.37 (dd, J = 5.2, |
| | | | | 12.8 Hz, 1H), 4.50-4.38 (m, 1H), 3.59 (s, 3H), |
| | | | | 3.05-2.95 (m, 2H), 2.94-2.80 (m, 1H), 2.76- |
| | | | | 2.67 (m, 1H), 2.65-2.58 (m, 1H), 2.25-2.20 (m, |
| | | | | 2H), 2.19-2.10 (m, 4H), 2.09-2.02 (m, 2H), 2.02- |
| | | | | 1.87 (m, 6H), 1.83-1.73 (m, 2H), 1.72-1.62 |
| | | | | (m, 1H), 1.21-1.08 (m, 2H) |
| I-129 | AZK | BVJ | 739.6 | 11.40 (s, 1H), 11.09 (s, 1H), 8.96 (d, J = 2.0 Hz, |
| | | | | 1H), 8.78 (d, J = 2.0 Hz, 1H), 8.44 (s, 1H), 8.25 |
| | | | | (d, J = 4.0 Hz, 1H), 8.13 (s, 1H), 7.69 (d, J = 9.2 |
| | | | | Hz, 1H), 7.39 (dd, J = 1.2, 8.8 Hz, 1H), 7.07-6.94 |
| | | | | (m, 3H), 6.93 (d, J = 4.0 Hz, 1H), 5.37 (dd, J = |
| | | | | 6.0, 12.0 Hz, 1H), 4.53-4.40 (m, 1H), 3.59 (s, |
| | | | | 3H), 3.05-2.96 (m, 2H), 2.94-2.84 (m, 1H), 2.77- |
| | | | | 2.68 (m, 1H), 2.65-2.59 (m, 1H), 2.24-2.16 (m, |
| | | | | 4H), 2.11-2.04 (m, 2H), 2.03-1.88 (m, 6H), 1.84- |
| | | | | 1.75 (m, 4H), 1.71-1.63 (m, 1H), 1.19-1.11 (m, 2H) |
| I-155 | BWB | ATJ | 768.4 | 10.56-10.47 (m, 2H), 9.48-9.36 (m, 1H), 8.72- |
| | | | | 8.66 (m, 1H), 8.49 (s, 1H), 8.48-8.44 (m, 1H), |
| | | | | 8.43-8.38 (m, 1H), 8.33 (d, J = 2.4 Hz, 1H), 8.22 |
| | | | | (d, J = 7.6 Hz, 1H), 7.64 (q, J = 8.0 Hz, 1H), 7.42 |
| | | | | (d, J = 1.2 Hz, 1H), 7.14 (s, 1H), 7.13-7.01 (m, |
| | | | | 1H), 4.76-4.47 (m, 1H), 4.45-4.31 (m, 1H), 3.98 |
| | | | | (s, 3H), 3.96-3.87 (m, 1H), 3.87-3.78 (m, 1H), |
| | | | | 3.73-3.63 (m, 1H), 3.29-3.04 (m, 2H), 3.00- |
| | | | | 2.89 (m, 1H), 2.76 (td, J = 5.2, 16.8 Hz, 1H), 2.56- |
| | | | | 2.51 (m, 1H), 2.49-2.44 (m, 3H), 2.15 (d, J = |
| | | | | 9.2 Hz, 3H), 2.09-1.80 (m, 5H), 1.79-1.57 (m, |
| | | | | 1H), 1.32-1.12 (m, 2H) |
| I-158 | BWC | ATJ | 786.3 | 11.11 (s, 1H), 10.50 (s, 1H), 8.70 (s, 1H), 8.48- |
| | | | | 8.45 (m, 1H), 8.43-8.38 (m, 1H), 8.37-8.35 (m, |
| | | | | 1H), 8.22 (d, J = 7.6 Hz, 1H), 7.53-7.39 (m, 1H), |
| | | | | 7.17-7.12 (m, 2H), 7.06-7.02 (m, 1H), 5.42- |
| | | | | 5.37 (m, 1H), 4.59-4.37 (m, 3H), 3.98 (s, 3H), |
| | | | | 3.86-3.76 (m, 2H), 3.60-3.55 (m, 5H), 3.13- |
| | | | | 3.02 (m, 1H), 2.94-2.90 (m, 1H), 2.76-2.70 (m, |
| | | | | 2H), 2.62-2.60 (s, 2H), 2.39-2.35 (m, 1H), 2.24- |
| | | | | 2.17 (m, 2H), 2.03-1.91 (m, 6H), 1.46-1.29 (m, 2H) |
| I-159 | AZK | BWF | 781.2 | 11.10 (s, 1H), 8.84 (s, 1H), 8.81 (d, J = 2.0 Hz, |
| | | | | 1H), 8.67 (d, J = 1.6 Hz, 1H), 8.56 (s, 1H), 8.52 |
| | | | | (d, J = 4.0 Hz, 1H), 8.33 (d, J = 6.8 Hz, 1H), 8.09 |
| | | | | (s, 1H), 7.09-6.96 (m, 3H), 6.88 (d, J = 3.6 Hz, |
| | | | | 1H), 5.38 (dd, J = 5.2, 12.4 Hz, 1H), 4.64-4.52 |
| | | | | (m, 1H), 3.94-3.83 (m, 1H), 3.61 (s, 3H), 3.30- |
| | | | | 3.29 (m, 2H), 2.94-2.84 (m, 1H), 2.77-2.69 (m, |
| | | | | 1H), 2.66-2.56 (m, 2H), 2.53-2.51 (m, 2H), 2.30- |
| | | | | 2.20 (m, 3H), 2.07-1.95 (m, 4H), 1.95-1.72 (m, |
| | | | | 7H), 1.36 (d, J = 6.4 Hz, 6H), 1.29-1.17 (m, 2H) |
| I-160 | BAI | BWH | 785.2 | 11.09 (s, 1H), 10.52 (s, 1H), 9.32 (d, J = 1.6 Hz, |
| | | | | 1H), 9.24 (d, J = 1.6 Hz, 1H), 8.77 (s, 1H), 8.56 |
| | | | | (s, 1H), 7.98 (s, 1H), 7.72 (s, 1H), 7.03-6.96 (m, |
| | | | | 1H), 6.96-6.92 (m, 1H), 6.91-6.86 (m, 1H), 5.35 |
| | | | | (dd, J = 4.8, 12.8 Hz, 1H), 4.58-4.46 (m, 1H), |
| | | | | 3.64 (s, 3H), 3.05-2.80 (m, 7H), 2.77-2.68 (m, |
| | | | | 1H), 2.65-2.56 (m, 2H), 2.28-2.24 (m, 2H), 2.23- |
| | | | | 2.13 (m, 3H), 2.03-1.95 (m, 4H), 1.95-1.90 |
| | | | | (m, 1H), 1.75-1.63 (m, 1H), 1.22-1.08 (m, 2H) |
| I-161 | BWL | BTW | 726.4 | 10.52 (s, 1H), 10.37 (s, 1H), 9.18-9.04 (m, 1H), |
| | | | | 8.46-8.22 (m, 5H), 8.21-8.16 (m, 1H), 7.93- |
| | | | | 7.79 (m, 1H), 7.78-7.68 (m, 1H), 7.65-7.59 (m, |
| | | | | 1H), 7.59-7.39 (m, 2H), 4.57-4.42 (m, 1H), 4.15- |
| | | | | 4.00 (m, 1H), 3.96-3.86 (m, 1H), 3.78-3.63 |
| | | | | (m, 2H), 3.27-3.05 (m, 4H), 3.00-2.90 (m, 1H), |
| | | | | 2.79-2.72 (m, 1H), 2.64-2.52 (m, 3H), 2.29- |
| | | | | 2.12 (m, 3H), 2.10-1.87 (m, 5H), 1.39-1.16 (m, 2H) |
| I-162 | BWQ | BRR | 772.4 | 10.20 (s, 1H), 9.07 (s, 1H), 8.61 (s, 1H), 8.51- |
| | | | | 8.46 (m, 2H), 8.41 (t, J = 8.0 Hz, 1H), 8.26-8.21 |
| | | | | (m, 1H), 6.90-6.78 (m, 2H), 5.46-5.36 (m, 1H), |
| | | | | 4.64-4.52 (m, 1H), 3.63-3.54 (m, 3H), 3.04- |
| | | | | 3.02 (m, 3H), 3.02-2.90 (m, 3H), 2.81-2.68 (m, |
| | | | | 2H), 2.52 (s, 1H), 2.46 (s, 3H), 2.29 (s, 1H), 2.27- |
| | | | | 2.15 (m, 6H), 2.09-1.91 (m, 8H), 1.81-1.76 |
| | | | | (m, 1H), 1.72-1.66 (m, 2H), 1.22-1.10 (m, 2H) |
| I-163^{c} | BWR | BTW | 758.4 | 11.10 (s, 1H), 10.36 (s, 1H), 8.41 (s, 1H), 8.39- |
| | | | | 8.30 (m, 3H), 8.18 (d, J = 7.2 Hz, 1H), 7.66-7.58 |
| | | | | (m, 1H), 7.58-7.51 (m, 1H), 7.10-6.83 (m, 3H), |
| | | | | 5.44-5.30 (m, 1H), 4.55-4.39 (m, 1H), 3.80- |
| | | | | 3.50 (m, 4H), 3.13-2.80 (m, 5H), 2.66-2.57 (m, |
| | | | | 2H), 2.27-2.09 (m, 4H), 2.09-1.81 (m, 6H), 1.72- |
| | | | | 1.56 (m, 1H), 1.45-1.00 (m, 6H) |
| I-164 | BWS | BTW | 770.3 | 11.10 (s, 1H), 10.37 (s, 1H), 8.44-8.34 (m, 3H), |
| | | | | 8.31 (s, 1H), 8.21-8.15 (m 1H), 7.67-7.59 (m, |
| | | | | 1H), 7.58-7.51 (m, 1H), 7.20-7.15 (m, 1H), 7.07 |
| | | | | (t, J = 6.8 Hz, 1H), 7.02-6.92 (m, 1H), 5.37 (dd, |
| | | | | J = 4.8, 12.4 Hz, 1H), 4.55-4.46 (m, 1H), 3.71 (s, |
| | | | | 3H), 3.05-2.98 (m, 2H), 2.94-2.85 (m, 3H), 2.75- |
| | | | | 2.70 (m, 2H), 2.68-2.58 (m, 5H), 2.28-2.13 |
| | | | | (m, 5H), 2.12-2.05 (m, 3H), 2.03-1.94 (m, 3H), |
| | | | | 1.40-1.22 (m, 2H) |
| I-165 | BAI | BVB | 731.3 | 11.09 (s, 1H), 9.90 (s, 1H), 9.31 (s, 1H), 9.21 (s, |
| | | | | 1H), 8.78 (s, 1H), 8.31 (s, 1H), 8.08 (s, 1H), 7.08 |
| | | | | (s, 1H), 7.03-6.97 (m, 1H), 6.96-6.89 (m, 2H), |
| | | | | 5.40-5.32 (m, 1H), 4.47-4.35 (m, 1H), 3.87 (s, |
| | | | | 3H), 3.64 (s, 3H), 3.29 (s, 3H), 3.02-2.83 (m, |
| | | | | 6H), 2.73-2.69 (m, 1H), 2.64-2.59 (m, 1H), 2.28- |
| | | | | 2.23 (m, 1H), 2.21-2.11 (m, 3H), 2.01-1.97 (m, |
| | | | | 3H), 1.95-1.87 (m, 2H), 1.76-1.58 (m, 1H), 1.24- |
| | | | | 1.11 (m, 2H) |
| I-166 | BDY | BVB | 743.3 | 11.09 (s, 1H), 9.91 (s, 1H), 9.31 (S, 1H), 9.20 (d, |
| | | | | J = 1.6 Hz, 1H), 8.78 (s, 1H), 8.30 (d, J = 10.4 Hz, |
| | | | | 2H), 7.08 (s, 1H), 7.00-6.91 (m, 2H), 6.81-6.79 |
| | | | | (m, 1H), 5.38-5.30 (m, 1H), 4.46-4.32 (m, 1H), |
| | | | | 3.87 (s, 3H), 3.76 (s, 1H), 3.63 (s, 3H), 3.48 (s, |
| | | | | 2H), 3.48-3.46 (m, 1H), 2.89 (d, J = 4.0 Hz, 1H), |
| | | | | 2.73-2.62 (m, 4H), 2.60-2.59 (m, 1H), 2.43- |
| | | | | 2.40 (m, 1H), 2.20-2.11 (m, 2H), 2.02-2.00 (m, |
| | | | | 2H), 1.92-1.79 (m, 4H), 1.78-1.62 (m, 1H), 1.55- |
| | | | | 1.41 (m, 1H), 1.22-1.09 (m, 2H) |
| I-167 | BWS | ATJ | 800.4 | 11.10 (s, 1H), 10.51 (s, 1H), 8.70 (s, 1H), 8.49- |
| | | | | 8.44 (m, 1H), 8.44-8.38 (m, 1H), 8.35 (s, 1H), |
| | | | | 8.25-8.20 (m, 1H), 7.16 (s, 2H), 7.10-6.93 (m, |
| | | | | 2H), 5.45-5.30 (m, 1H), 4.49-4.35 (m, 1H), 3.99 |
| | | | | (s, 3H), 3.71 (s, 3H), 3.10-2.80 (m, 4H), 2.76- |
| | | | | 2.68 (m, 1H), 2.65-2.59 (m, 1H), 2.53-2.52 (m, |
| | | | | 3H), 2.31-2.04 (m, 8H), 2.03-1.46 (m, 6H), 1.39- |
| | | | | 1.18 (m, 2H) |
| I-168 | BWT | ATJ | 786.7 | 11.09 (s, 1H), 10.51 (s, 1H), 8.70 (s, 1H), 8.48- |
| | | | | 8.44 (m, 1H), 8.43-8.38 (m, 1H), 8.35 (s, 1H), |
| | | | | 8.22 (d, J = 7.6 Hz, 1H), 7.15 (s, 1H), 7.05-6.96 |
| | | | | (m, 2H), 6.87 (d, J = 3.6 Hz, 1H), 5.35 (dd, J = |
| | | | | 5.2, 12.4 Hz, 1H), 4.46-4.37 (m, 1H), 3.98 (s, |
| | | | | 3H), 3.63 (s, 3H), 3.26 (s, 2H), 2.97-2.82 (m, |
| | | | | 2H), 2.77-2.56 (m, 7H), 2.25-2.13 (m, 3H), 2.09- |
| | | | | 1.86 (m, 7H), 1.30-1.20 (m, 2H) |
| I-169 | AZL | BVB | 730.7 | 11.08 (s, 1H), 9.91 (s, 1H), 9.31 (d, J = 1.8 Hz, |
| | | | | 1H), 9.21 (d, J = 1.8 Hz, 1H), 8.78 (s, 1H), 8.31 |
| | | | | (s, 1H), 8.08 (s, 1H), 7.10 (d, J = 9.2 Hz, 2H), 7.01 |
| | | | | (d, J = 8.0 Hz, 1H), 6.95-6.91 (m, 1H), 5.34 (dd, |
| | | | | J = 5.4, 12.8 Hz, 1H), 4.46-4.32 (m, 1H), 3.88 (s, |
| | | | | 3H), 3.34 (s, 3H), 3.06 (d, J = 7.6 Hz, 2H), 2.96- |
| | | | | 2.83 (m, 2H), 2.73 (d, J = 8.4 Hz, 3H), 2.31 (s, |
| | | | | 1H), 2.16 (d, J = 10.8 Hz, 4H), 2.04-1.87 (m, |
| | | | | 6H), 1.78 (s, 4H), 1.21-1.05 (m, 2H) |
| I-171 | BSN | BWU | 743.3 | 10.54 (s, 1H), 10.43 (s, 1H), 9.64 (s, 1H), 9.04 (s, |
| | | | | 1H), 8.96 (s, 1H), 8.58 (s, 1H), 8.52-8.47 (m, |
| | | | | 1H), 8.46-8.40 (m, 1H), 8.29-8.23 (m, 1H), 7.91- |
| | | | | 7.86 (m, 1H), 7.80 (t, J = 7.2 Hz, 1H), 7.68-7.61 |
| | | | | (m, 1H), 3.98-3.88 (m, 1H), 3.85-3.50 (m, 2H), |
| | | | | 3.31-3.26 (m, 2H), 3.21-3.13 (m, 1H), 3.10- |
| | | | | 2.85 (m, 2H), 2.81-2.72 (m, 1H), 2.53-2.51 (m, |
| | | | | 3H), 2.29-2.20 (m, 2H), 2.19-1.74 (m, 7H), 1.74- |
| | | | | 1.59 (m, 2H), 1.30-1.05 (m, 2H) |
| I-172^{d} | BWW | BTW | 811.3 | 11.10 (s, 1H), 10.36 (s, 1H), 8.43-8.33 (m, 3H), |
| | | | | 8.30 (s, 1H), 8.17 (d, J = 7.2 Hz, 1H), 7.64-7.59 |
| | | | | (m, 1H), 7.58-7.52 (m, 1H), 7.13-7.08 (m, 1H), |
| | | | | 7.07-6.97 (m, 2H), 5.42-5.34 (m, 1H), 4.50- |
| | | | | 4.40 (m, 1H), 3.70-3.59 (m, 2H), 3.55 (s, 3H), |
| | | | | 3.02-2.97 (m, 1H), 2.94-2.84 (m, 2H), 2.77- |
| | | | | 2.70 (m, 2H), 2.65-2.57 (m, 2H), 2.24-2.13 (m, |
| | | | | 3H), 2.04-1.94 (m, 6H), 1.94-1.83 (m, 3H), 1.74- |
| | | | | 1.62 (m, 2H) |
| I-173 | BWY | ATJ | 787.2 | 11.07 (s, 1H), 10.50 (s, 1H), 8.69 (s, 1H), 8.49- |
| | | | | 8.37 (m, 2H), 8.34 (s, 1H), 8.22 (d, J = 8.4 Hz, |
| | | | | 1H), 7.15 (s, 1H), 6.92 (s, 2H), 5.33 (dd, J = 5.2, |
| | | | | 12.8 Hz, 1H), 4.45-4.33 (m, 1H), 3.98 (s, 3H), |
| | | | | 3.61 (s, 3H), 3.23-3.07 (m, 3H), 2.98-2.81 (m, |
| | | | | 2H), 2.74-2.62 (m, 2H), 2.56 (s, 3H), 2.40-2.36 |
| | | | | (m, 1H), 2.19-2.13 (m, 2H), 2.06-1.86 (m, 6H), |
| | | | | 1.85-1.55 (m, 6H), 1.24-1.11 (m, 2H) |
| I-176 | AZK | BXE | 773.3 | 11.09 (s, 1H), 10.24 (s, 1H), 8.43-8.35 (m, 2H), |
| | | | | 8.34 (s, 1H), 8.18 (dd, J = 1.2, 7.6 Hz, 1H), 7.93 |
| | | | | (d, J = 1.6 Hz, 1H), 7.05-7.00 (m, 3H), 7.00- |
| | | | | 6.96 (m, 1H), 5.37 (dd, J = 5.6, 12.8 Hz, 1H), 4.47- |
| | | | | 4.36 (m, 1H), 3.93 (s, 3H), 3.59 (s, 3H), 2.99 (br |
| | | | | d, J = 11.2 Hz, 2H), 2.92-2.85 (m, 1H), 2.78- |
| | | | | 2.68 (m, 1H), 2.65-2.58 (m, 1H), 2.21 (br d, J = |
| | | | | 7.2 Hz, 2H), 2.15 (br d, J = 12.4 Hz, 2H), 2.10- |
| | | | | 2.04 (m, 2H), 2.01-1.96 (m, 3H), 1.93 (br d, J = |
| | | | | 12.4 Hz, 2H), 1.82-1.74 (m, 4H), 1.74-1.60 (m, |
| | | | | 2H), 1.19-1.10 (m, 2H) |
| I-177 | BWX | ATJ | 807.3 | 11.13 (s, 1H), 10.51 (s, 1H), 8.70 (s, 1H), 8.49- |
| | | | | 8.45 (m, 1H), 8.44-8.39 (m, 1H), 8.34 (s, 1H), |
| | | | | 8.23 (d, J = 8.0 Hz, 1H), 7.16 (s, 1H), 7.14-7.04 |
| | | | | (m, 2H), 5.44-5.37 (m, 1H), 4.45-4.33 (m, 1H), |
| | | | | 3.99 (s, 3H), 3.67 (s, 3H), 3.30 (s, 4H), 3.17-3.00 |
| | | | | (m, 2H), 2.93-2.83 (m, 1H), 2.18-2.14 (m, 2H), 2.07- |
| | | | | 1.89 (m, 7H), 1.85-1.67 (m, 5H), 1.26-1.10 (m, 3H) |
| I-178 | BWX | BRR | 778.5 | 11.12 (s, 1H), 10.20 (s, 1H), 9.06 (s, 1H), 8.61 (s, |
| | | | | 1H), 8.52-8.47 (m, 2H), 8.45-8.37 (m, 1H), 8.23 |
| | | | | (d, J = 8.0 Hz, 1H), 7.16-7.02 (m, 2H), 5.40 (dd, |
| | | | | J = 6.0, 12.8 Hz, 1H), 4.66-4.53 (m, 1H), 3.66 (s, |
| | | | | 3H), 3.29 (s, 3H), 2.88 (d, J = 2.4 Hz, 1H), 2.77- |
| | | | | 2.69 (m, 1H), 2.61 (s, 1H), 2.22 (d, J = 10.4 Hz, |
| | | | | 4H), 2.01 (d, J = 5.6 Hz, 7H), 1.86-1.77 (m, 5H), |
| | | | | 1.26-1.16 (m, 2H) |
| I-179 | AQK | BVU | 786.7 | 11.09 (s, 1H), 8.83 (d, J = 2.0 Hz, 1H), 8.71 (d, J = |
| | | | | 2.0 Hz, 1H), 8.68 (s, 1H), 8.45 (d, J = 7.6 Hz, |
| | | | | 1H), 8.27 (d, J = 7.6 Hz, 1H), 8.05 (s, 1H), 7.82 |
| | | | | (d, J = 4.8 Hz, 1H), 7.10 (d, J = 4.8 Hz, 1H), 6.99- |
| | | | | 6.95 (m, 1H), 6.89-6.85 (m, 1H), 5.35 (dd, J = |
| | | | | 5.6, 12.4 Hz, 1H), 3.83-3.70 (m, 2H), 3.64 (s, |
| | | | | 3H), 3.15 (d, J = 11.0 Hz, 2H), 2.94-2.82 (m, 1H), |
| | | | | 2.75-2.63 (m, 4H), 2.42 (s, 2H), 2.27-2.22 (m, |
| | | | | 5H), 1.99 (s, 1H), 1.91-1.75 (m, 7H), 1.66 (s, |
| | | | | 2H), 1.39-1.32 (m, 2H), 1.27 (d, J = 6.4 Hz, 6H), |
| | | | | 0.94 (m, J = 11.5 Hz, 2H) |
| I-180 | BWX | BTW | 777.6 | 11.11 (s, 1H), 10.36 (s, 1H), 8.42-8.32 (m, 3H), |
| | | | | 8.29 (s, 1H), 8.17 (d, J = 8.0 Hz, 1H), 7.64-7.59 |
| | | | | (m, 1H), 7.57-7.52 (m, 1H), 7.12-7.06 (m, 2H), |
| | | | | 5.44.5.34 (m, 1H), 4.50-4.40 (m, 1H), 3.66 (s, |
| | | | | 3H), 3.03-2.94 (m, 3H), 2.94-2.83 (m, 1H), 2.71 |
| | | | | (d, J = 4.4 Hz, 1H), 2.66-2.58 (m, 1H), 2.24- |
| | | | | 2.14 (m, 4H), 2.02 (d, J = 10.4 Hz, 3H), 2.00-1.88 |
| | | | | (m, 4H), 1.77-1.63 (m, 5H), 1.20-1.10 (m, 2H) |
| I-181 | BRY | BH | 742.5 | 10.62 (s, 1H), 10.54 (s, 1H), 9.27 (s, 1H), 8.62 (d, |
| | | | | J = 1.6 Hz, 1H), 8.49 (s, 1H), 8.43-8.35 (m, 2H), |
| | | | | 8.19 (dd, J = 1.2, 7.6 Hz, 1H), 8.06 (s, 1H), 7.96- |
| | | | | 7.88 (m, 3H), 7.78 (dd, J = 1.6, 8.8 Hz, 1H), 3.98- |
| | | | | 3.90 (m, 1H), 3.76-3.69 (m, 1H), 3.29-3.19 |
| | | | | (m, 3H), 3.14-3.06 (m, 1H), 3.01-2.92 (m, 1H), |
| | | | | 2.91-2.83 (m, 1H), 2.80-2.73 (m, 1H), 2.68- |
| | | | | 2.54 (m, 2H), 2.39-2.31 (m, 1H), 2.25-2.18 (m, |
| | | | | 2H), 2.00-1.89 (m, 6H), 1.80-1.73 (m, 1H), 1.69- |
| | | | | 1.59 (m, 2H), 1.21-1.10 (m, 2H) |
| I-182 | AZK | BXF | 756.6 | 11.09 (s, 1H), 10.05 (s, 1H), 9.05 (s, 1H), 8.84 (d, |
| | | | | J = 2.4 Hz, 1H), 8.42 (s, 1H), 8.36 (s, 1H), 7.65 (s, |
| | | | | 1H), 7.05-6.96 (m, 3H), 5.40-5.34 (m, 1H), 3.92 |
| | | | | (s, 3H), 3.58 (s, 3H), 3.26-3.21 (m, 1H), 3.10- |
| | | | | 3.05 (m, 1H), 2.99 (d, J = 10.8 Hz, 2H), 2.93-2.85 |
| | | | | (m, 1H), 2.75-2.69 (m, 1H), 2.64-2.58 (m, 1H), |
| | | | | 2.22-2.16 (m, 4H), 2.10-2.05 (m, 2H), 2.00- |
| | | | | 1.93 (m, 3H), 1.82-1.72 (m, 4H), 1.66-1.56 (m, |
| | | | | 3H), 1.19-1.03 (m, 2H) |
| I-183 | AZK | BVU | 757.7 | 11.09 (s, 1H), 8.84 (d, J = 2.0 Hz, 1H), 8.70 (d, J = |
| | | | | 2.0 Hz, 1H), 8.68 (s, 1H), 8.46 (d, J = 6.9 Hz, |
| | | | | 1H), 8.28 (d, J = 8.0 Hz, 1H), 8.16 (s, 1H), 8.05 |
| | | | | (s, 1H), 7.84 (d, J = 4.8 Hz, 1H), 7.10 (d, J = 4.9 |
| | | | | Hz, 1H), 7.04-7.00 (m, 2H), 7.00 (d, J = 4.1 Hz, |
| | | | | 1H), 5.36 (dd, J = 5.5, 12.5 Hz, 1H), 3.82-3.75 |
| | | | | (m, 1H), 3.58 (s, 3H), 3.00 (d, J = 9.6 Hz, 2H), |
| | | | | 2.91-2.83 (m, 1H), 2.76-2.62 (m, 2H), 2.20 (d, |
| | | | | J = 6.3 Hz, 2H), 2.13-2.05 (m, 2H), 2.02-1.96 |
| | | | | (m, 1H), 1.92-1.74 (m, 10H), 1.36 (d, J = 10.0 |
| | | | | Hz, 2H), 1.28 (d, J = 6.4 Hz, 6H), 1.02-0.92 (m, 2H) |
| I-184 | BSN | BVU | 739.7 | 10.53 (s, 1H), 9.62 (s, 1H), 8.83 (s, 1H), 8.68 (d, |
| | | | | J = 7.6 Hz, 2H), 8.57 (s, 1H), 8.44 (d, J = 7.0 Hz, |
| | | | | 1H), 8.30 (d, J = 7.9 Hz, 1H), 8.14 (s, 1H), 8.06 |
| | | | | (s, 1H), 7.89-7.85 (m, 1H), 7.83-7.77 (m, 2H), |
| | | | | 7.64 (d, J = 6.6 Hz, 1H), 7.10 (d, J = 4.9 Hz, 1H), |
| | | | | 3.92 (d, J = 4.9, 10.9 Hz, 1H), 3.83-3.67 (m, 5H), |
| | | | | 3.19-3.14 (m, 2H), 3.02-2.94 (m, 1H), 2.76 (d, |
| | | | | J = 5.3, 17.0 Hz, 1H), 1.98-1.86 (m, 10H), 1.64- |
| | | | | 1.56 (m, 1H), 1.36 (d, J = 10.6 Hz, 2H), 1.28 (d, J = |
| | | | | 6.1 Hz, 6H), 1.04 (d, J = 10.3 Hz, 2H) |
| I-185 | AZK | BXG | 747.6 | 11.09 (s, 1H), 10.13 (s, 1H), 9.32 (s, 1H), 9.22 (s, |
| | | | | 1H), 8.79 (s, 1H), 8.41 (s, 1H), 7.66 (s, 1H), 7.05- |
| | | | | 6.95 (m, 3H), 5.39-5.35 (m, 1H), 3.93 (s, 3H), |
| | | | | 3.59 (s, 3H), 3.24-3.22 (m, 1H), 3.10-3.03 (m, |
| | | | | 1H), 2.99 (d, J = 10.4 Hz, 2H), 2.91-2.84 (m, |
| | | | | 1H), 2.74-2.57 (m, 3H), 2.22-2.18 (m, 4H), 2.09- |
| | | | | 2.05 (m, 2H), 1.97-1.94 (m, 3H), 1.79 (s, 4H), |
| | | | | 1.65-1.58 (m, 2H), 1.15-1.06 (m, 2H) |
| I-189 | AZK | BXI | 768.3 | 11.10 (s, 1H), 10.65 (s, 1H), 8.64 (s, 1H), 8.49- |
| | | | | 8.36 (m, 3H), 8.27-8.21 (m, 2H), 7.07-6.94 (m, |
| | | | | 3H), 5.44-5.34 (m, 1H), 4.63-4.52 (m, 1H), 3.60 |
| | | | | (s, 3H), 3.26-3.18 (m, 1H), 3.07-2.97 (m, 2H), |
| | | | | 2.94-2.84 (m, 1H), 2.75-2.60 (m, 2H), 2.27- |
| | | | | 2.16 (m, 4H), 2.15-2.05 (m, 2H), 2.04-1.91 (m, |
| | | | | 5H), 1.85-1.65 (m, 5H), 1.24-1.10 (m, 2H) |
| I-190 | BSN | BUR | 739.6 | 10.54 (s, 1H), 9.64 (s, 1H), 8.81 (d, J = 2.0 Hz, |
| | | | | 1H), 8.68 (d, J = 2.0 Hz, 1H), 8.61-8.56 (m, 3H), |
| | | | | 8.52 (d, J = 4.0 Hz, 1H), 8.36 (d, J = 7.6 Hz, 1H), |
| | | | | 8.08 (s, 1H), 7.92-7.85 (m, 1H), 7.80 (t, J = 7.6 |
| | | | | Hz, 1H), 7.64 (d, J = 6.4 Hz, 1H), 6.89 (d, J = 4.0 |
| | | | | Hz, 1H), 3.97-3.87 (m, 1H), 3.83-3.63 (m, 5H), 3.86- |
| | | | | 2.87 (m, 2H), 2.83-2.71 (m, 2H), 2.52 (s, 2H), 2.06- |
| | | | | 1.82 (m, 9H), 1.71-1.60 (m, 1H), 1.39 (q, J = 11.7 |
| | | | | Hz, 2H), 1.29 (d, J = 6.3 Hz, 6H), 1.13-1.00 (m, 2H) |
| I-191 | BSN | BCN | 772.5 | 10.55 (s, 1H), 10.51 (s, 1H), 9.70 (s, 1H), 9.02 (s, |
| | | | | 1H), 8.61 (s, 1H), 8.49-8.39 (m, 2H), 8.26-8.21 |
| | | | | (m, 1H), 7.95-7.90 (m, 1H), 7.86-7.81 (m, 1H), |
| | | | | 7.70 (s, 1H), 7.63 (d, J = 7.2 Hz, 1H), 4.03 (s, |
| | | | | 3H), 4.00-3.89 (m, 2H), 3.75-3.67 (m, 2H), 3.67- |
| | | | | 3.63 (m, 1H), 3.31-3.21 (m, 2H), 3.17-3.11 (m, |
| | | | | 1H), 3.10-3.05 (m, 2H), 3.03-2.95 (m, 1H), 2.81- |
| | | | | 2.73 (m, 1H), 2.54-2.52 (m, 1H), 2.27-2.21 |
| | | | | (m, 2H), 2.20-2.10 (m, 3H), 2.06-1.91 (m, 3H), |
| | | | | 1.75-1.61 (m, 2H), 1.34-1.19 (m, 2H) |
| I-192 | BSN | BTO | 772.6 | 10.51 (s, 1H), 9.86 (s, 1H), 9.59 (s, 1H), 8.55 (s, |
| | | | | 1H), 8.29 (s, 1H), 8.22-8.14 (m, 1H), 8.12-8.05 |
| | | | | (m, 1H), 7.99-7.90 (m, 2H), 7.87-7.81 (m, 1H), |
| | | | | 7.76 (d, J = 7.6 Hz, 1H), 7.64 (d, J = 6.8 Hz, |
| | | | | 1H), 7.06 (s, 1H), 4.43-4.33 (m, 1H), 3.96-3.87 (m, |
| | | | | 1H), 3.84 (s, 3H), 3.74-3.65 (m, 1H), 3.62-3.53 |
| | | | | (m, 1H), 3.08-2.99 (m, 2H), 2.98-2.91 (m, 1H), |
| | | | | 2.81-2.69 (m, 1H), 2.31 (d, J = 1.6, 3.6 Hz, 1H), |
| | | | | 2.28-2.19 (m, 4H), 2.18-2.10 (m, 2H), 1.99 (d, |
| | | | | J = 1.6, 13.9 Hz, 2H), 1.93-1.83 (m, 5H), 1.72- |
| | | | | 1.60 (m, 1H), 1.19-1.06 (m, 2H) |
| I-195 | BUC | BVB | 760.6 | 11.07 (s, 1H), 9.90 (s, 1H), 9.33-9.19 (m, 1H), |
| | | | | 8.86-8.72 (m, 1H), 8.33-8.28 (m, 1H), 8.24- |
| | | | | 8.07 (m, 1H), 7.09 (s, 1H), 7.06-6.98 (m, 1H), |
| | | | | 6.90 (d, J = 8.6 Hz, 1H), 6.72 (d, J = 8.8 Hz, |
| | | | | 1H), 5.32 (dd, J = 5.4, 12.8 Hz, 1H), 4.46-4.31 |
| | | | | (m, 1H), 3.90-3.85 (m, 3H), 3.77 (s, 3H), 3.56 (s, |
| | | | | 3H), 3.00 (d, J = 9.2 Hz, 2H), 2.93-2.82 (m, 1H), |
| | | | | 2.72 (d, J = 4.4 Hz, 1H), 2.65-2.56 (m, 1H), 2.44- |
| | | | | 2.36 (m, 2H), 2.24 (d, J = 6.0 Hz, 2H), 2.16 (d, J = |
| | | | | 9.8 Hz, 2H), 2.07-1.86 (m, 8H), 1.73-1.63 (m, 1H), |
| | | | | 1.6 (d, J = 11.9 Hz, 2H), 1.21-1.09 (m, 2H) |
| I-197 | BXL | BWF | 764.5 | 10.54 (s, 1H), 9.45 (s, 1H), 8.85 (s, 1H), 8.81 (d, |
| | | | | J = 2.0 Hz, 1H), 8.67 (d, J = 2.0 Hz, 1H), 8.58- |
| | | | | 8.50 (m, 3H), 8.34 (d, J = 7.2 Hz, 1H), 8.09 (s, |
| | | | | 1H), 7.78-7.70 (m, 1H), 7.65 (d, J = 7.6 Hz, 1H), |
| | | | | 7.31 (d, J = 7.2 Hz, 1H), 6.88 (d, J = 3.6 Hz, |
| | | | | 1H), 4.66-4.53 (m, 1H), 3.97-3.82 (m, 2H), 3.70 (td, |
| | | | | J = 6.0, 12.0 Hz, 1H), 3.30-3.08 (m, 5H), 2.97 |
| | | | | (ddd, J = 6.0, 10.0, 16.4 Hz, 2H), 2.76 (td, J = 5.6, |
| | | | | 16.8 Hz, 2H), 2.52 (s, 3H), 2.25 (d, J = 10.8 Hz, |
| | | | | 2H), 2.03 (d, J = 12.0 Hz, 2H), 1.97-1.84 (m, |
| | | | | 2H), 1.83-1.68 (m, 1H), 1.36 (d, J = 6.4 Hz, 6H), |
| | | | | 1.30-1.15 (m, 2H) |
| I-198 | BRY | BVU | 739.4 | 10.54 (s, 1H), 9.27 (s, 1H), 8.83 (d, J = 2.0 Hz, |
| | | | | 1H), 8.73-8.66 (m, 2H), 8.50 (s, 1H), 8.45 (d, J = |
| | | | | 6.8 Hz, 1H), 8.31 (d, J = 7.2 Hz, 1H), 8.13 (s, |
| | | | | 1H), 8.06 (s, 2H), 7.94 (d, J = 8.4 Hz, 1H), 7.86- |
| | | | | 7.72 (m, 2H), 7.10 (d, J = 4.8 Hz, 1H), 3.90-3.97 |
| | | | | (m, 1H), 3.85-3.63 (m, 4H), 2.92 (s, 1H), 2.90- |
| | | | | 2.81 (m, 1H), 2.73-2.79 (m, 1H), 2.04-1.78 (m, 10H), |
| | | | | 1.75-1.49 (m, 2H), 1.42-1.32 (m, 2H), 1.28 (d, J = |
| | | | | 6.4 Hz, 6H), 1.20-1.11 (m, 1H), 1.08-0.97 (m, 2H) |
| I-199 | BXN | BVU | 811.8 | 11.13 (s, 1H), 8.84 (d, J = 2.4 Hz, 1H), 8.71 (d, J = |
| | | | | 2.0 Hz, 1H), 8.68 (s, 1H), 8.43 (d, J = 7.2 Hz, |
| | | | | 1H), 8.31 (d, J = 7.6 Hz, 1H), 8.05 (s, 1H), 7.82 |
| | | | | (d, J = 4.8 Hz, 1H), 7.19 (d, J = 8.0 Hz, 1H), 7.15- |
| | | | | 7.09 (m, 2H), 7.07-7.01 (m, 1H), 5.40 (dd, J = |
| | | | | 5.2, 12.8 Hz, 1H), 4.53 (s, 2H), 3.85-3.67 (m, |
| | | | | 3H), 3.65 (s, 3H), 2.95-2.84 (m, 2H), 2.77-2.62 |
| | | | | (m, 3H), 2.52 (s, 2H), 2.45-2.36 (m, 2H), 2.02 (m, |
| | | | | 3H), 1.94-1.79 (m, 5H), 1.75-1.60 (m, 2H), 1.43-1.30 |
| | | | | (m, 2H), 1.27 (d, J = 6.4 Hz, 6H), 1.11-0.97 (m, 2H) |
| I-200^{b} | BUC | BVH | 730.2 | 11.0 (s, 1H), 10.59 (s, 1H), 8.44-8.36 (m, 2H), |
| | | | | 8.35-8.25 (m, 3H), 7.61 (d, J = 1.0 Hz, 2H), 6.91 |
| | | | | (d, J = 8.4 Hz, 1H), 6.72 (d, J = 8.8 Hz, 1H), 5.32 |
| | | | | (dd, J = 5.2, 12.4 Hz, 1H), 4.51-4.39 (m, 1H), |
| | | | | 3.77 (s, 3H), 3.56 (s, 3H), 2.98 (d, J = 10.4 Hz, |
| | | | | 2H), 2.92-2.84 (m, 1H), 2.71-2.59 (m, 2H), 2.42 |
| | | | | (d, J = 11.2 Hz, 2H), 2.24-2.15 (m, 4H), 2.07- |
| | | | | 1.87 (m, 8H), 1.72-1.64 (m, 1H), 1.58 (d, J = 10.8 |
| | | | | Hz, 2H), 1.20-1.10 (m, 2H) |
| I-201 | BSN | BH | 742.6 | 10.62 (s, 1H), 10.54 (s, 1H), 9.62 (s, 1H), 8.61 (s, |
| | | | | 1H), 8.57 (s, 1H), 8.44-8.34 (m, 2H), 8.19 (d, J = |
| | | | | 7.6 Hz, 1H), 8.15 (s, 1H), 7.96-7.86 (m, 2H), |
| | | | | 7.79 (t, J = 7.6 Hz, 1H), 7.65 (d, J = 7.2 Hz, 1H), |
| | | | | 3.97-3.88 (m, 1H), 3.71 (td, J = 6.0, 12.0 Hz, |
| | | | | 1H), 3.64 (d, J = 4.0 Hz, 1H), 3.18-3.04 (m, 3H), |
| | | | | 3.03-2.93 (m, 1H), 2.76 (td, J = 5.2, 16.4 Hz, |
| | | | | 1H), 2.47-2.35 (m, 4H), 2.21 (d, J = 11.2 Hz, 2H), |
| | | | | 2.05-1.82 (m, 6H), 1.72 (d, J = 0.8 Hz, 1H), 1.69- |
| | | | | 1.56 (m, 2H), 1.20-1.06 (m, 2H) |
| I-202 | BWY | BTW | 757.3 | 11.07 (s, 1H), 10.35 (s, 1H), 8.42-8.33 (m, 3H), |
| | | | | 8.30 (s, 1H), 8.17 (dd, J = 1.2, 7.6 Hz, 1H), 7.65- |
| | | | | 7.58 (m, 1H), 7.58-7.51 (m, 1H), 6.97-6.86 (m, |
| | | | | 2H), 5.33 (dd, J = 5.2, 12.4 Hz, 1H), 4.51-4.39 |
| | | | | (m, 1H), 3.61 (s, 3H), 3.06-2.97 (m, 2H), 2.95- |
| | | | | 2.67 (m, 3H), 2.65-2.58 (m, 1H), 2.55 (s, 3H), |
| | | | | 2.26 (d, J = 7.2 Hz, 2H), 2.20-2.08 (m, 4H), 2.03- |
| | | | | 1.89 (m, 5H), 1.74-1.60 (m, 5H), 1.21-1.07 (m, 2H) |
| I-203 | BQM | BXI | 782.4 | 11.11-11.04 (m, 1H), 10.64 (s, 1H), 8.63 (s, 1H), |
| | | | | 8.47-8.37 (m, 3H), 8.26-8.21 (m, 2H), 6.90- |
| | | | | 6.78 (m, 2H), 5.33 (dd, J = 5.2, 12.4 Hz, 1H), 4.57 |
| | | | | (t, J = 11.6 Hz, 1H), 3.62-3.53 (m, 3H), 3.23- |
| | | | | 3.15 (m, 1H), 3.05-2.96 (m, 2H), 2.93-2.81 (m, |
| | | | | 1H), 2.75-2.59 (m, 2H), 2.46 (s, 3H), 2.29 (s, |
| | | | | 1H), 2.27-2.17 (m, 5H), 2.06 (d, J = 10.8 Hz, |
| | | | | 2H), 1.98 (t, J = 11.6 Hz, 5H), 1.78 (s, 1H), 1.69 |
| | | | | (d, J = 10.4 Hz, 2H), 1.23-1.08 (m, 2H) |
| I-205 | BQM | BXI | 798.5 | 11.08 (s, 1H), 10.65 (s, 1H), 8.64 (s, 1H), 8.50- |
| | | | | 8.35 (m, 3H), 8.26-8.21 (m, 2H), 6.94 (d, J = 8.8 |
| | | | | Hz, 1H), 6.75 (d, J = 8.8 Hz, 1H), 5.33 (dd, J = |
| | | | | 5.2, 12.4 Hz, 1H), 4.67-4.54 (m, 1H), 3.79 (s, |
| | | | | 3H), 3.60 (s, 3H), 3.51-3.42 (m, 1H), 3.14-3.05 |
| | | | | (m, 1H), 2.94-2.81 (m, 2H), 2.78-2.52 (m, 6H), |
| | | | | 2.22 (d, J = 9.2 Hz, 2H), 2.09-1.92 (m, 6H), 1.91- |
| | | | | 1.82 (m, 1H), 1.80-1.66 (m, 2H), 1.26 (dd, J = |
| | | | | 1.6, 10.0 Hz, 2H), 1.17 (t, J = 7.2 Hz, 1H) |
| I-206^{h} | BXQ | BVB | 771.2 | 9.31 (d, J = 1.6 Hz, 1H), 9.04 (d, J = 1.6 Hz, 1H), |
| | | | | 8.74 (d, J = 11.6 Hz, 2H), 8.51 (s, 1H), 8.31-8.11 |
| | | | | (m, 1H), 7.88 (s, 1H), 7.09 (s, 1H), 7.04-6.96 (m, |
| | | | | 1H), 6.87 (d, J = 8.0 Hz, 1H), 6.59 (d, J = 7.6 Hz, |
| | | | | 1H), 5.23-5.18 (m, J = 5.2, 12.4 Hz, 1H), 4.35- |
| | | | | 4.29 (m, J = 3.6, 8.0, 11.6 Hz, 1H), 4.02 (s, 3H), |
| | | | | 3.74 (s, 3H), 3.64-3.62 (m, J = 4.4 Hz, 2H), 3.15- |
| | | | | 3.08 (m, 2H), 2.99-2.91 (m, 2H), 2.89-2.83 |
| | | | | (m, 3H), 2.80-2.68 (m, 4H), 2.34-2.31 (m, J = |
| | | | | 10.8 Hz, 2H), 2.25-2.14 (m, 3H), 2.09-2.06 (m, |
| | | | | J = 11.6 Hz, 2H), 2.01-1.89 (m, 4H), 1.85-1.75 |
| | | | | (m, 1H), 1.35-1.22 (m, 2H) |
| I-207^{h} | BXR | BVB | 799.2 | 9.31 (d, J = 2.0 Hz, 1H), 9.04 (d, J = 2.0 Hz, 1H), |
| | | | | 8.74 (d, J = 11.2 Hz, 2H), 8.51 (t, J = 2.0 Hz, 1H), |
| | | | | 8.25-8.13 (m, 1H), 7.89 (s, 1H), 7.09 (s, 1H), |
| | | | | 7.04-6.99 (m, 1H), 6.96-6.92 (m, 1H), 6.59 (d, |
| | | | | J = 7.6 Hz, 1H), 5.23-5.18 (m, J = 5.2, 12.4 Hz, |
| | | | | 1H), 4.39-4.28 (m, 1H), 4.02 (s, 3H), 3.76 (s, |
| | | | | 3H), 3.06-2.87 (m, 10H), 2.80-2.70 (m, 4H), 2.35- |
| | | | | 2.32 (m, J = 11.2 Hz, 2H), 2.27-2.16 (m, 2H), |
| | | | | 2.14-2.11 (m, J = 12.4 Hz, 2H), 2.06-1.96 (m, |
| | | | | 4H), 1.79 (s, 3H), 1.72-1.65 (m, 2H), 1.37-1.26 (m, 2H). |
| I-208 | BWL | ATJ | 756.1 | 10.51 (d, J = 2.0 Hz, 2H), 9.34-9.17 (m, 1H), 9.13 |
| | | | | (s, 1H), 8.70 (s, 1H), 8.50-8.38 (m, 2H), 8.35 (s, |
| | | | | 1H), 8.22 (d, J = 7.6 Hz, 1H), 7.88 (d, J = 8.4 Hz, |
| | | | | 1H), 7.73 (d, J = 8.4 Hz, 1H), 7.56 (s, 1H), 7.15 |
| | | | | (s, 1H), 4.51-4.37 (m, 1H), 4.19-4.01 (m, 2H), |
| | | | | 3.99 (s, 3H), 3.95-3.86 (m, 1H), 3.82-3.59 (m, |
| | | | | 3H), 3.28-3.09 (m, 6H), 3.01-2.89 (m, 1H), 2.80- |
| | | | | 2.71 (m, 1H), 2.25-2.14 (m, 2H), 2.07-1.87 |
| | | | | (m, 5H), 1.40-1.18 (m, 2H) |
| I-209 | BAI | BTW | 744.6 | 11.26-10.96 (m, 1H), 10.37 (s, 1H), 8.42-8.34 |
| | | | | (m, 3H), 8.33-8.30 (m, 2H), 8.18 (d, J = 1.2 Hz, |
| | | | | 7.6 Hz, 1H), 7.65-7.60 (m, 1H), 7.58-7.53 (m, |
| | | | | 1H), 7.02-6.84 (m, 3H), 5.36 (s, 1H), 4.52-4.41 |
| | | | | (m, 1H), 3.64 (s, 3H), 3.03-2.86 (m, 6H), 2.78- |
| | | | | 2.58 (m, 4H), 2.26 (d, J = 7.2 Hz, 2H), 2.18 (d, J = |
| | | | | 10.4 Hz, 2H), 2.04-1.89 (m, 5H), 1.68 (d, J = |
| | | | | 6.8 Hz, 1H), 1.27-1.03 (m, 4H) |
| I-210 | BWY | BRR | 758.3 | 11.07 (s, 1H), 10.20 (s, 1H), 9.06 (s, 1H), 8.61 (s, |
| | | | | 1H), 8.51-8.45 (m, 2H), 8.45-8.38 (m, 1H), 8.23 |
| | | | | (d, J = 8.0 Hz, 1H), 6.98-6.85 (m, 2H), 5.32 (dd, |
| | | | | J = 52, 12.4 Hz, 1H), 4.65-4.51 (m, 1H), 3.61 (s, |
| | | | | 3H), 3.00 (d, J = 10.8 Hz, 2H), 2.95-2.83 (m, |
| | | | | 1H), 2.82-2.72 (m, 1H), 2.66-2.54 (m, 5H), 2.26- |
| | | | | 2.17 (m, 4H), 2.13-2.05 (m, 2H), 1.98 (t, J = |
| | | | | 11.2 Hz, 5H), 1.67 (s, 5H), 1.22-1.09 (m, 2H) |
| I-211 | BXL | ATJ | 756.5 | 10.52 (d, J = 13.2 Hz, 2H), 9.46 (s, 1H), 8.70 (s, |
| | | | | 1H), 8.55 (s, 1H), 8.49-8.44 (m, 1H), 8.43-8.37 |
| | | | | (m, 1H), 8.34 (s, 1H), 8.22 (d, J = 7.6 Hz, 1H), |
| | | | | 7.80-7.60 (m, 2H), 7.31 (s, 1H), 7.16 (s, 1H), |
| | | | | 4.41 (s, 1H), 4.03-3.86 (m, 4H), 3.73-3.67 (m, |
| | | | | J = 5.6, 12.0 Hz, 1H), 3.62-3.43 (m, 1H), 3.31- |
| | | | | 3.08 (m, 5H), 3.01-2.93 (m, 1H), 2.82-2.62 (m, |
| | | | | 3H), 2.58-2.52 (m, 2H), 2.19-2.16 (m, J = 10.4 |
| | | | | Hz, 2H), 2.06-1.69 (m, 5H), 1.33-1.08 (m, 2H) |
| I-212 | AZK | BXS | 797.4 | 11.09 (s, 1H), 10.11 (s, 1H), 9.36 (d, J = 1.6 Hz, |
| | | | | 1H), 9.21 (d, J = 1.6 Hz, 1H), 8.80 (s, 1H), 8.30 |
| | | | | (s, 1H), 8.25 (s, 1H), 7.65 (s, 1H), 7.08-6.88 (m, |
| | | | | 4H), 5.37 (dd, J = 5.2, 12.4 Hz, 1H), 4.47 (s, 1H), |
| | | | | 4.37 (s, 1H), 4.30 (s, 1H), 4.01 (d, J = 7.2 Hz, 1H), |
| | | | | 3.72 (d, J = 7.6 Hz, 1H), 3.59 (s, 3H), 2.99 (d, J = |
| | | | | 9.2 Hz, 3H), 2.93-2.85 (m, 1H), 2.76-2.68 (m, |
| | | | | 1H), 2.62 (d, J = 17.6 Hz, 1H), 2.21 (d, J = 6.8 Hz, |
| | | | | 2H), 2.18-2.12 (m, 2H), 2.11-2.03 (m, 3H), 2.02- |
| | | | | 1.95 (m, 3H), 1.93-1.85 (m, 3H), 1.84-1.72 |
| | | | | (m, 6H), 1.70-1.63 (m, 1H), 1.19-1.06 (m, 2H) |
| I-213 | BSN | BXI | 750.4 | 10.64 (s, 1H), 10.53 (s, 1H), 9.61 (s, 1H), 8.64 (s, |
| | | | | 1H), 8.57 (s, 1H), 8.47-8.36 (m, 3H), 8.27-8.20 |
| | | | | (m, 2H), 7.89-7.83 (m, 1H), 7.78 (t, J = 7.6 Hz, |
| | | | | 1H), 7.66 (d, J = 7.2 Hz, 1H), 4.64-4.54 (m, 1H), |
| | | | | 3.92 (ddd, J = 5.2, 9.8, 12.0 Hz, 1H), 3.72 (td, J = |
| | | | | 6.0, 12.1 Hz, 1H), 3.62 (t, J = 15.2 Hz, 1H), 3.07 |
| | | | | (d, J = 10.0 Hz, 2H), 2.98 (ddd, J = 6.0, 10.0, 16.5 |
| | | | | Hz, 1H), 2.76 (td, J = 5.4, 16.6 Hz, 1H), 2.52 (s, |
| | | | | 1H), 2.31 (dd, J = 2.4, 8.0 Hz, 3H), 2.24-2.17 (m, 2H), |
| | | | | 2.07-1.84 (m, 8H), 1.78-1.65 (m, 1H), 1.25-1.11 (m, 2H) |
| I-216^{h} | BAI | BXX | 791.4 | 9.36 (d, J = 14.0 Hz, 1H), 8.83 (s, 1H), 8.42 (t, J = |
| | | | | 6.4 Hz, 1H), 8.05 (s, 1H), 7.88 (s, 1H), 7.81 (t, |
| | | | | J = 6.4 Hz, 1H), 7.44 (t, J = 7.6 Hz, 1H), 7.08 (s, |
| | | | | 1H), 7.03 (d, J = 6.8 Hz, 1H), 6.68-6.58 (m, 1H), |
| | | | | 5.21 (dd, J = 5.2, 12.8 Hz, 1H), 4.43-4.28 (m, |
| | | | | 1H), 4.02 (s, 3H), 3.77 (s, 3H), 3.18-3.03 (m, |
| | | | | 3H), 3.01-2.72 (m, 6H), 2.36 (d, J = 11.6 Hz, 3H), |
| | | | | 2.27-2.20 (m, 2H), 2.18-2.10 (m, 1H), 2.07-1.96 (m, |
| | | | | 2H), 1.67-1.59 (m, 4H), 1.19 (d, J = 5.2 Hz, 2H) |
| I-217 | AQK | BXS | 826.4 | 11.09 (s, 1H), 10.06 (s, 1H), 9.36 (d, J = 1.6 Hz, |
| | | | | 1H), 9.21 (d, J = 1.6 Hz, 1H), 8.80 (s, 1H), 8.25 |
| | | | | (s, 1H), 7.65 (s, 1H), 7.00-6.86 (m, 4H), 5.35 (dd, |
| | | | | J = 5.2, 12.8 Hz, 1H), 4.47 (s, 1H), 4.42-4.33 (m, |
| | | | | 1H), 4.31 (s, 1H), 4.01 (d, J = 7.2 Hz, 1H), 3.72 |
| | | | | (d, J = 6.4 Hz, 1H), 3.64 (s, 3H), 3.43 (d, J = 8.0 |
| | | | | Hz, 2H), 3.19-3.15 (m, 2H), 2.99 (d, J = 9.6 Hz, |
| | | | | 1H), 2.95-2.83 (m, 2H), 2.77-2.70 (m, 3H), 2.63 |
| | | | | (d, J = 3.6 Hz, 1H), 2.17-2.12 (m, 2H), 2.07 (s, |
| | | | | 3H), 2.00-1.95 (m, 3H), 1.92 (d, J = 3.6 Hz, 1H), |
| | | | | 1.89 (d, J = 8.0 Hz, 2H), 1.86-1.81 (m, 2H), 1.77 |
| | | | | (d, J = 10.4 Hz, 2H), 1.71-1.66 (m, 2H), 1.63- |
| | | | | 1.58 (m, 1H), 1.18-1.09 (m, 2H) |
| I-218 | BAI | BXE | 774.4 | 11.09 (s, 1H), 10.24 (s, 1H), 8.42-8.38 (m, 1H), |
| | | | | 8.37-8.33 (m, 2H), 8.16 (d, J = 7.6 Hz, 1H), 7.94 |
| | | | | (s, 1H), 7.02 (s, 1H), 6.99-6.93 (m, 2H), 6.90 (dd, |
| | | | | J = 2.4, 3.6 Hz, 1H), 5.36 (dd, J = 4.8, 12.4 Hz, |
| | | | | 1H), 4.52-4.38 (m, 1H), 3.93 (s, 3H), 3.64 (s, |
| | | | | 3H), 3.03-2.84 (m, 6H), 2.67-2.58 (m, 2H), 2.30- |
| | | | | 2.22 (m, 2H), 2.20-2.12 (m, 3H), 2.05-1.87 |
| | | | | (m, 7H), 1.77-1.59 (m, 1H), 1.26-1.09 (m, 2H) |
| I-219 | BXY | ATJ | 782.2 | 10.51 (d, J = 8.4 Hz, 2H), 9.52 (s, 1H), 8.69 (s, |
| | | | | 1H), 8.53 (s, 1H), 8.48-8.44 (m, 1H), 8.43-8.38 |
| | | | | (m, 1H), 8.35 (s, 1H), 8.21 (d, J = 7.6 Hz, 1H), |
| | | | | 7.74-7.68 (m, 1H), 7.64-7.59 (m, 1H), 7.32 (d, |
| | | | | J = 7.6 Hz, 1H), 7.16 (s, 1H), 4.45-4.33 (m, 1H), |
| | | | | 3.98 (s, 3H), 3.91 (ddd, J = 5.2, 9.6, 12.4 Hz, 1H), |
| | | | | 3.70 (td, J = 6.0, 12.0 Hz, 1H), 3.23-3.05 (m, |
| | | | | 5H), 3.01-2.92 (m, 1H), 2.75 (td, J = 5.6, 16.8 |
| | | | | Hz, 1H), 2.34-2.27 (m, 2H), 2.20-2.03 (m, 9H), |
| | | | | 2.00-1.84 (m, 3H), 1.24-1.13 (m, 2H) |
| I-220 | BXZ | ATJ | 768.2 | 10.51 (d, J = 12.0 Hz, 2H), 9.52 (s, 1H), 8.68 (s, |
| | | | | 1H), 8.49 (s, 1H), 8.48-8.44 (m, 1H), 8.43-8.37 |
| | | | | (m, 1H), 8.32 (s, 1H), 8.26-8.19 (m, 1H), 7.78- |
| | | | | 7.72 (m, 1H), 7.59 (dd, J = 2.4, 8.0 Hz, 2H), 7.15 |
| | | | | (s, 1H), 4.37 (d, J = 3.2 Hz, 1H), 3.98 (s, 3H), 3.95- |
| | | | | 3.85 (m, 2H), 3.80 (d, J = 11.2 Hz, 1H), 3.75- |
| | | | | 3.71 (m, 1H), 3.70-3.66 (m, 2H), 3.49 (d, J = 11.2 |
| | | | | Hz, 2H), 3.02-2.91 (m, 1H), 2.79-2.72 (m, 1H), |
| | | | | 2.20-2.10 (m, 3H), 2.02-1.99 (m, 2H), 1.95- |
| | | | | 1.82 (m, 2H), 1.55-1.43 (m, 1H), 1.28-1.13 (m, 2H) |
| I-221 | BSN | BXE | 755.4 | 10.54 (s, 1H), 10.24 (s, 1H), 9.61 (s, 1H), 8.57 (s, |
| | | | | 1H), 8.43-8.38 (m, 1H), 8.37-8.33 (m, 2H), 8.20- |
| | | | | 8.15 (m, 1H), 7.96 (d, J = 1.2 Hz, 1H), 7.88- |
| | | | | 7.84 (m, 1H), 7.78 (t, J = 7.6 Hz, 1H), 7.68 (d, J = |
| | | | | 7.2 Hz, 1H), 7.04 (d, J = 1.2 Hz, 1H), 4.47-4.38 |
| | | | | (m, 1H), 3.93 (s, 3H), 3.92-3.88 (m, 1H), 3.72 (d, |
| | | | | J = 6.0, 12.4 Hz, 1H), 3.66-3.57 (m, 1H), 3.07 (d, |
| | | | | J = 11.1 Hz, 2H), 3.02-2.94 (m, 1H), 2.80-2.72 |
| | | | | (m, 1H), 2.52 (s, 1H), 2.32 (d, J = 5.6 Hz, 3H), |
| | | | | 2.20-2.13 (m, 2H), 2.04-1.98 (m, 2H), 1.97- |
| | | | | 1.85 (m, 6H), 1.76-1.66 (m, 1H), 1.24-1.11 (m, 2H) |
| I-222 | BXL | BXE | 756.4 | 10.53 (s, 1H), 10.24 (s, 1H), 9.43 (s, 1H), 8.53 (s, |
| | | | | 1H), 8.43-8.38 (m, 1H), 8.37-8.33 (m, 2H), 8.16 |
| | | | | (d, J = 7.2 Hz, 1H), 7.92 (d, J = 1.2 Hz, 1H), 7.77- |
| | | | | 7.69 (m, 1H), 7.64 (d, J = 8.4 Hz, 1H), 7.28 (d, J = |
| | | | | 7.2 Hz, 1H), 7.04 (d, J = 1.2 Hz, 1H), 4.48-4.39 |
| | | | | (m, 1H), 3.94-3.93 (m, 3H), 3.92-3.86 (m, 1H), |
| | | | | 3.70 (d, J = 6.0, 12.0 Hz, 1H), 3.22-3.10 (m, 5H), |
| | | | | 3.00-2.92 (m, 1H), 2.80-2.75 (m, 1H), 2.75- |
| | | | | 2.68 (m, 4H), 2.21-2.13 (m, 2H), 2.00 (d, J = 15.6 |
| | | | | Hz, 2H), 1.97-1.88 (m, 2H), 1.78-1.64 (m, 2H), |
| | | | | 1.24-1.10 (m, 2H) |
| I-223 | BXL | BCN | 773.3 | 10.56 (s, 1H), 10.52 (s, 1H), 9.52 (s, 1H), 9.02 (s, |
| | | | | 1H), 8.60 (s, 1H), 8.50-8.39 (m, 2H), 8.25 (d, J = |
| | | | | 8.4 Hz, 1H), 7.82-7.69 (m, 3H), 7.39 (d, J = 7.2 |
| | | | | Hz, 1H), 4.04 (s, 3H), 3.93 (m, 1H), 3.78-3.65 |
| | | | | (m, 4H), 3.35-3.08 (m, 8H), 2.98 (m, 1H), 2.82- |
| | | | | 2.73 (m, 1H), 2.25 (d, J = 11.6 Hz, 2H), 2.01 (d, J = |
| | | | | 10.8 Hz, 3H), 1.75-1.62 (m, 2H), 1.34-1.22 (m, 2H) |
| I-224 | BXL | AGL | 784.4 | 1.11-1.34 (m, 2 H) 1.62 (s, 6 H)2.01 (s, 5 H)2.19 |
| | | | | (d, J = 9.6 Hz, 2 H) 2.52 (s, 2 H) 2.76 (dt, J = 16.8, |
| | | | | 5.2 Hz, 2H) 2.97 (s, 2 H) 3.05-3.29 (m, 5 H) 3.44- |
| | | | | 3.61 (m, 1 H) 3.70 (dt, J = 12.0, 6.0 Hz, 1 H) |
| | | | | 3.85-3.97 (m, 1 H) 4.48 (s, 1 H) 5.95 (s, 1 H) 7.32 |
| | | | | (dd, J = 4.0, 1.2 Hz, 1 H) 7.58 (s, 1 H) 7.60-7.70 |
| | | | | (m, 1 H) 7.71-7.78 (m, 1 H) 8.17 (s, 1 H) 8.34-8.40 |
| | | | | (m, 2 H) 8.44 (s, 1 H) 8.56 (s, 1 H) 8.72 (s, 1 H) |
| | | | | 9.46 (s, 1 H) 10.54 (s, 1 H) 12.37 (s, 1 H) |
| I-225 | BWT | AGL | 814.4 | 12.36 (s, 1H), 11.09 (s, 1H), 8.72 (s, 1H), 8.49- |
| | | | | 8.41 (m, 1H), 8.41-8.33 (m, 2H), 8.19-8.14 (m, |
| | | | | 1H), 7.57 (s, 1H), 6.98 (d, J = 4.4 Hz, 2H), 6.90- |
| | | | | 6.81 (m, 1H), 5.94 (s, 1H), 5.43-5.25 (m, 1H), |
| | | | | 4.58-4.31 (m, 1H), 3.86 (s, 1H), 3.63 (d, J = 1.2 |
| | | | | Hz, 3H), 3.57 (d, J = 10.0 Hz, 1H), 3.13 (d, J = 8.0 |
| | | | | Hz, 1H), 3.03-2.75 (m, 4H), 2.71 (dd, J = 5.2, 7.2 |
| | | | | Hz, 1H), 2.62 (d, J = 16.0 Hz, 2H), 2.17 (d, J = |
| | | | | 12.0 Hz, 2H), 2.08-1.86 (m, 8H), 1.62 (s, 7H), |
| | | | | 1.21 (J = 7.6, 12.0 Hz, 2H) |
| I-226 | BWR | AGL | 816.3 | 12.36 (s, 1H), 11.09 (s, 1H), 8.72 (s, 1H), 8.48- |
| | | | | 8.42 (m, 1H), 8.40-8.32 (m, 2H), 8.21-8.10 (m, |
| | | | | 1H), 7.57 (s, 1H), 7.03-6.86 (m, 3H), 5.95 (s, |
| | | | | 1H), 5.36 (dd, J = 5.4, 12.8 Hz, 1H), 4.54-4.35 |
| | | | | (m, 1H), 3.64 (s, 3H), 3.21-2.98 (m, 4H), 2.97- |
| | | | | 2.78 (m, 3H), 2.71-2.58 (m, 3H), 2.16 (s, 3H), |
| | | | | 2.03-1.84 (m, 5H), 1.62 (s, 7H), 1.40-0.97 (m, 6H) |
| I-227 | BXY | AGL | 810.2 | 12.36 (s, 1H), 10.53 (s, 1H), 9.53 (s, 1H), 8.72 (s, |
| | | | | 1H), 8.54 (s, 1H), 8.47-8.43 (m, 1H), 8.40-8.32 |
| | | | | (m, 2H), 8.18-8.12 (m, 1H), 7.75-7.69 (m, 1H), |
| | | | | 7.64 (d, J = 8.4 Hz, 1H), 7.57 (s, 1H), 7.35 (d, J = |
| | | | | 7.2 Hz, 1H), 5.94 (s, 1H), 4.52-4.39 (m, 1H), 3.91 |
| | | | | (ddd, J = 5.2, 9.6, 12.4 Hz, 1H), 3.70 (td, J = 6.0, |
| | | | | 12.0 Hz, 1H), 3.29-3.07 (m, 6H), 2.96 (ddd, J = |
| | | | | 6.0, 10.0, 16.4 Hz, 1H), 2.76 (td, J = 5.6, 16.4 Hz, |
| | | | | 1H), 2.55-2.51 (m, 4H), 2.22-2.04 (m, 7H), 1.95 |
| | | | | (q, J = 12.0 Hz, 2H), 1.62 (s, 6H), 1.31-1.13 (m, 2H) |
| I-228 | BXZ | AGL | 796.2 | 12.36 (s, 1H), 10.54 (s, 1H), 9.54 (s, 1H), 8.71 (s, |
| | | | | 1H), 8.51 (s, 1H), 8.47-8.42 (m, 1H), 8.41-8.32 |
| | | | | (m, 2H), 8.16 (d, J = 7.6 Hz, 1H), 7.80-7.74 (m, |
| | | | | 1H), 7.62 (d, J = 8.0 Hz, 2H), 7.57 (s, 1H), 5.95 |
| | | | | (s, 1H), 4.50-4.36 (m, 1H), 3.96-3.81 (m, 4H), |
| | | | | 3.71 (td, J = 6.0, 12.4 Hz, 1H), 3.65-3.45 (m, |
| | | | | 6H), 2.97 (ddd, J = 6.0, 10.0, 16.4 Hz, 1H), 2.76 |
| | | | | (td, J = 5.2, 16.8 Hz, 1H), 2.52 (d, J = 2.0 Hz, 2H), |
| | | | | 2.15 (d, J = 10.8 Hz, 2H), 2.05-1.85 (m, 4H), 1.61 |
| | | | | (s, 6H), 1.32-1.17 (m, 2H) |
| I-229 | BWB | AGL | 796.4 | 12.36 (s, 1H), 10.52 (d, J = 6.2 Hz, 1H), 9.42 (d, |
| | | | | J = 12.4 Hz, 1H), 8.71 (s, 1H), 8.50-8.33 (m, 4H), |
| | | | | 8.16 (d, J = 7.6 Hz, 1H), 7.63 (q, J = 8.0 Hz, 1H), |
| | | | | 7.56 (s, 1H), 7.47-7.32 (m, 1H), 7.14-6.98 (m, |
| | | | | 1H), 5.94 (s, 1H), 4.66-4.50 (m, 1H), 4.49-4.37 |
| | | | | (m, 1H), 3.98-3.89 (m, 1H), 3.86-3.78 (m, 1H), |
| | | | | 3.69 (td, J = 6.0, 11.9 Hz, 2H), 3.46-3.37 (m, 2H), |
| | | | | 2.99-2.91 (m, 1H), 2.76 (td, J = 5.2, 16.6 Hz, |
| | | | | 2H), 2.59 (s, 1H), 2.21-2.09 (m, 3H), 2.04-1.86 |
| | | | | (m, 5H), 1.75-1.65 (m, 1H), 1.61 (s, 6H), 1.33- |
| | | | | 1.09 (m, 3H) |
| I-230 | CAB | AGL | 798.4 | 12.36 (s, 1H), 10.54 (s, 1H), 9.57-9.43 (m, 1H), |
| | | | | 8.72 (s, 1H), 8.60-8.52 (m, 1H), 8.46-8.43 (m, |
| | | | | 1H), 8.39-8.34 (m, 2H), 8.18-8.15 (m, 1H), 7.77- |
| | | | | 7.61 (m, 2H), 7.57 (s, 1H), 7.40-7.24 (m, 1H), |
| | | | | 5.94 (s, 1H), 4.53-4.42 (m, 1H), 3.95-3.88 (m, |
| | | | | 1H), 3.73-3.67 (m, 1H), 3.63-3.52 (m, 1H), 3.24- |
| | | | | 3.17 (m, 2H), 3.16-3.03 (m, 2H), 3.00-2.93 |
| | | | | (m, 1H), 2.80-2.72 (td, J = 5.6, 16.8 Hz, 2H), |
| | | | | 2.26-2.07 (m, 5H), 2.03-1.87 (m, 4H), 1.62 (s, |
| | | | | 6H), 1.49-1.35 (m, 2H), 1.26-1.06 (m, 4H) |
| I-231 | BAI | BWF | 782.4 | 11.09 (s, 1H), 8.84 (s, 1H), 8.81 (s, 1H), 8.67 (s, |
| | | | | 1H), 8.55 (s, 1H), 8.52 (d, J = 3.6 Hz, 1H), 8.34 |
| | | | | (d, J = 7.2 Hz, 1H), 8.09 (s, 1H), 7.04-6.89 (m, |
| | | | | 3H), 6.88 (d, J = 3.6 Hz, 1H), 5.36 (dd, J = 5.6, |
| | | | | 12.8 Hz, 1H), 4.57 (t, J = 10.8 Hz, 1H), 3.95-3.80 |
| | | | | (m, 1H), 3.64 (s, 3H), 3.31-3.24 (m, 2H), 3.21- |
| | | | | 2.78 (m, 7H), 2.76-2.63 (m, 2H), 2.31-2.13 (m, 4H), |
| | | | | 2.06-1.95 (m, 3H), 1.95-1.82 (m, 2H), 1.78-1.59 (m, |
| | | | | 1H), 1.36 (d, J = 6.4 Hz, 6H), 1.28-1.09 (m, 2H) |
| I-232 | BWC | AGL | 814.4 | 12.36 (s, 1H), 11.11 (s, 1H), 8.71 (s, 1H), 8.49- |
| | | | | 8.41 (m, 1H), 8.40-8.32 (m, 2H), 8.16 (d, J = 7.2 |
| | | | | Hz, 1H), 7.57 (s, 1H), 7.33 (d, J = 8.0 Hz, 1H), |
| | | | | 7.10 (t, J = 8.0 Hz, 1H), 6.97 (d, J = 7.6 Hz, 1H), |
| | | | | 5.94 (s, 1H), 5.45-5.30 (m, 1H), 4.49-4.41 (m, |
| | | | | 1H), 3.77-3.65 (m, 2H), 3.62 (d, J = 11.2 Hz, 2H), |
| | | | | 3.57 (s, 3H), 3.14-3.04 (m, 2H), 2.95-2.85 (m, |
| | | | | 1H), 2.77-2.69 (m, 1H), 2.65 (m, 1H), 2.61 (s, 2H), |
| | | | | 2.16 (d, J = 10.4 Hz, 2H), 2.03 (d, J = 8.0 Hz, |
| | | | | 4H), 1.98-1.85 (m, 3H), 1.62 (s, 6H), 1.58-1.50 |
| | | | | (m, 1H), 1.31-1.20 (m, 2H) |
| I-233 | CAC | ATJ | 770.3 | 10.55 (s, 1H), 10.51 (s, 1H), 9.83-9.25 (m, 1H), |
| | | | | 8.70 (s, 1H), 8.58 (s, 1H), 8.50-8.44 (m, 1H), |
| | | | | 8.44-8.37 (m, 1H), 8.35 (s, 1H), 8.25-8.20 (m, |
| | | | | 1H), 7.92-7.69 (m, 2H), 7.69-7.46 (m, 1H), 7.15 |
| | | | | (s, 1H), 4.50-4.36 (m, 1H), 3.99 (s, 3H), 3.95- |
| | | | | 3.86 (m, 1H), 3.81-3.60 (m, 3H), 3.60-3.47 (m, 1H), |
| | | | | 3.26-3.06 (m, 3H), 2.98 (d, J = 6.0 Hz, 2H), 2.88- |
| | | | | 2.69 (m, 2H), 2.20 (d, J = 6.4 Hz, 2H), 2.14-1.86 |
| | | | | (m, 5H), 1.43-0.94 (m, 3H), 0.89 (t, J = 7.2 Hz, 3H) |
| I-234 | CAC | AGL | 798.3 | 12.37 (s, 1H), 10.55 (s, 1H), 9.83-9.23 (m, 1H), |
| | | | | 8.73 (s, 1H), 8.58 (s, 1H), 8.47-8.42 (m, 1H), |
| | | | | 8.40-8.33 (m, 2H), 8.16 (d, J = 7.6 Hz, 1H), 7.93- |
| | | | | 7.70 (m, 2H), 7.57 (s, 2H), 5.95 (s, 1H), 4.49 (d, |
| | | | | J = 1.6 Hz, 1H), 4.03-3.85 (m, 1H), 3.82-3.41 |
| | | | | (m, 4H), 3.24-3.06 (m, 2H), 3.05-2.90 (m, 2H), |
| | | | | 2.86-2.69 (m, 2H), 2.52 (d, J = 2.0 Hz, 1H), 2.19 |
| | | | | (s, 3H), 2.02 (d, J = 10.4 Hz, 5H), 1.62 (s, 6H), |
| | | | | 1.40-1.14 (m, 2H), 0.90 (t, J = 6.8 Hz, 3H) |
| I-236^{h} | BTH | BAX | 872.4 | 10.88 (s, 1H), 7.60 (d, J = 8.4 Hz, 2H), 7.44 (s, |
| | | | | 1H), 7.43-7.37 (m, 1H), 7.15 (d, J = 8.4 Hz, 2H), |
| | | | | 5.18 (s, 1H), 4.31-4.16 (m, 2H), 3.70-3.66 (m, |
| | | | | 4H), 2.85-2.76 (m, 2H), 2.70-2.63 (m, 1H), 1.83 |
| | | | | (d, J = 12.4 Hz, 2H), 1.73-1.67 (m, 6H), 1.63- |
| | | | | 1.56 (m, 2H), 1.49 (s, 9H) |
| I-237 | CAE | CAG | 773.3 | 11.08 (s, 1H), 10.51 (s, 1H), 8.73-8.67 (m, 1H), |
| | | | | 8.48-8.43 (m, 1H), 8.43-8.35 (m, 2H), 8.20 (d, |
| | | | | J = 7.6 Hz, 1H), 7.16 (s, 1H), 7.04-6.93 (m, 3H), |
| | | | | 5.35 (dd, J = 5.2, 12.4 Hz, 1H), 4.83-4.70 (m, |
| | | | | 1H), 3.98 (s, 3H), 3.58 (s, 3H), 3.49-3.45 (m, |
| | | | | 2H), 3.27-3.14 (m, 5H), 3.03 (s, 2H), 2.94-2.83 |
| | | | | (m, 2H), 2.71-2.64 (m, 2H), 2.35 (s, 1H), 2.04- |
| | | | | 1.95 (m, 4H), 1.89 (d, J = 11.6 Hz, 2H), 1.67-1.55 |
| | | | | (m, 2H), 1.29-1.22 (m, 2H) |
| I-238 | AQK | BRR | 773.4 | 11.09 (s, 1H), 10.20 (s, 1H), 9.06 (s, 1H), 8.61 (s, |
| | | | | 1H), 8.52-8.45 (m, 2H), 8.45-8.38 (m, 1H), 8.32- |
| | | | | 8.22 (m, 1H), 7.02-6.94 (m, 1H), 6.94-6.84 |
| | | | | (m, 2H), 5.35 (dd, J = 5.6, 12.8 Hz, 1H), 4.66- |
| | | | | 4.51 (m, 1H), 3.65 (s, 3H), 3.17 (d, J = 11.2 Hz, |
| | | | | 2H), 2.95-2.83 (m, 1H), 2.80-2.56 (m, 5H), 2.48- |
| | | | | 2.25 (m, 5H), 2.20 (d, J = 9.2 Hz, 2H), 2.06- |
| | | | | 1.91 (m, 5H), 1.83 (s, 2H), 1.78-1.58 (m, 3H), |
| | | | | 1.25-1.08 (m, 2H) |
| I-239 | AQK | BXI | 797.6 | 11.09 (s, 1H), 10.64 (s, 1H), 8.64 (s, 1H), 8.49- |
| | | | | 8.35 (m, 3H), 8.29-8.20 (m, 2H), 7.04-6.79 (m, |
| | | | | 3H), 5.35 (dd, J = 5.4, 12.8 Hz, 1H), 4.62-4.51 |
| | | | | (m, 1H), 3.64 (s, 3H), 3.16 (d, J = 11.2 Hz, 2H), |
| | | | | 2.94-2.84 (m, 1H), 2.77-2.60 (m, 4H), 2.30 (d, |
| | | | | J = 6.8 Hz, 2H), 2.26 (s, 3H), 2.19 (d, J = 9.6 Hz, |
| | | | | 2H), 2.07-1.88 (m, 6H), 1.80 (d, J = 11.2 Hz, 2H), |
| | | | | 1.73-1.55 (m, 3H), 1.20-1.05 (m, 2H) |
| I-240 | AZK | CAH | 761.4 | 11.11 (s, 1H), 10.46 (s, 1H), 8.56 (d, J = 2.8 Hz, |
| | | | | 1H), 8.43-8.32 (m, 2H), 8.18-8.11 (m, 2H), 7.55 |
| | | | | (dd, J = 0.8, 13.2 Hz, 1H), 7.05-6.94 (m, 3H), |
| | | | | 5.42-5.31 (m, 1H), 4.56-4.45 (m, 1H), 3.60 (s, |
| | | | | 3H), 3.09 (d, J = 8.8 Hz, 3H), 2.92-2.85 (m, 1H), |
| | | | | 2.75-2.60 (m, 2H), 2.33-2.32 (m, 2H), 2.25- |
| | | | | 2.16 (m, 5H), 2.02-1.94 (m, 5H), 1.83 (s, 3H), |
| | | | | 1.75-1.70 (m, 1H), 1.22-1.12 (m, 2H) |
| I-241 | BRY | BWU | 743.3 | 10.54 (s, 1H), 10.42 (s, 1H), 9.28 (s, 1H), 9.04 (s, |
| | | | | 1H), 8.96 (s, 1H), 8.54-8.47 (m, 2H), 8.46-8.38 |
| | | | | (m, 1H), 8.28-8.22 (m, 1H), 8.06 (s, 1H), 7.95 (d, |
| | | | | J = 8.8 Hz, 1H), 7.78 (d, J = 8.4 Hz, 1H), 3.99- |
| | | | | 3.89 (m, 1H), 3.72 (td, J = 6.0, 12.0 Hz, 1H), 3.31- |
| | | | | 3.26 (m, 2H), 3.21-3.13 (m, 1H), 3.04-2.90 |
| | | | | (m, 2H), 2.89-2.70 (m, 2H), 2.54-2.51 (m, 3H), |
| | | | | 2.28-2.19 (m, 2H), 2.13-1.77 (m, 7H), 1.74- |
| | | | | 1.59 (m, 2H), 1.30-1.06 (m, 2H) |
| I-242 | AZK | CAI | 743.4 | 11.09 (s, 1H), 10.61 (s, 1H), 8.55 (d, J = 7.6 Hz, |
| | | | | 1H), 8.42-8.33 (m, 2H), 8.22-8.18 (m, 2H), 7.35- |
| | | | | 7.18 (m, 1H), 7.08-6.90 (m, 3H), 6.38 (s, 1H), |
| | | | | 5.37 (dd, J = 5.2, 12.4 Hz, 1H), 3.59 (s, 3H), 2.98 |
| | | | | (d, J = 10.8 Hz, 2H), 2.93-2.85 (m, 1H), 2.77- |
| | | | | 2.68 (m, 2H), 2.65-2.58 (m, 1H), 2.19 (d, J = 6.8 |
| | | | | Hz, 2H), 2.11-1.98 (m, 5H), 1.96-1.89 (m, 2H), |
| | | | | 1.78 (s, 4H), 1.68-1.39 (m, 4H), 1.12-0.99 (m, 2H) |
| I-243 | BWR | ATJ | 788.3 | 11.11 (s, 1H), 10.51 (s, 1H), 8.92-8.78 (m, 1H), |
| | | | | 8.70 (s, 1H), 8.48-8.45 (m, 1H), 8.43-8.39 (m, |
| | | | | 1H), 8.22 (d, J = 8.4 Hz, 1H), 7.14 (s, 1H), 7.08- |
| | | | | 7.02 (m, 1H), 7.01-6.93 (m, 2H), 5.38 (dd, J = |
| | | | | 5.4, 12.8 Hz, 1H), 4.51-4.34 (m, 1H), 3.99 (s, |
| | | | | 3H), 3.76 (d, J = 11.6 Hz, 1H), 3.65 (s, 3H), 3.62- |
| | | | | 3.52 (m, 1H), 3.27 (s, 1H), 3.21-2.81 (m, 5H), 2.78- |
| | | | | 2.53 (m, 3H), 2.46-2.35 (m, 1H), 2.21 (d, J = 11.6 |
| | | | | Hz, 2H), 2.13-1.82 (m, 6H), 1.55-1.23 (m, 5H) |
| I-244 | CAJ | BVB | 757.4 | 11.10 (s, 1H), 9.90 (s, 1H), 9.30 (s, 1H), 9.20 (d, J = |
| | | | | 1.6 Hz, 1H), 8.77 (s, 1H), 8.29 (s, 1H), 8.07 (s, |
| | | | | 1H), 7.12-7.01 (m, 2H), 7.00-6.87 (m, 2H), 5.36 |
| | | | | (dd, J = 5.2, 12.4 Hz, 1H), 4.41-4.29 (m, 1H), |
| | | | | 3.87 (s, 3H), 3.76 (d, J = 2.4 Hz, 2H), 3.63 (s, 3H), |
| | | | | 3.54 (s, 4H), 3.27 (s, 4H), 2.99-2.81 (m, 2H), |
| | | | | 2.76-2.68 (m, 1H), 2.66-2.53 (m, 2H), 2.11 (d, |
| | | | | J = 9.6 Hz, 2H), 2.03-1.96 (m, 1H), 1.91-1.80 |
| | | | | (m, 4H), 1.50-1.37 (m, 1H), 1.19-1.06 (m, 2H) |
| I-245 | CAK | BRR | 788.4 | 10.20 (s, 1H), 9.07 (s, 1H), 8.62 (s, 1H), 8.51- |
| | | | | 8.47 (m, 2H), 8.42 (t, J = 7.6 Hz, 1H), 8.24 (d, J = |
| | | | | 7.6 Hz, 1H), 6.94-6.87 (m, 1H), 6.71 (d, J = 8.8 |
| | | | | Hz, 1H), 5.39 (dd, J = 5.6, 12.8 Hz, 1H), 4.64- |
| | | | | 4.54 (m, 1H), 3.77 (s, 3H), 3.57 (s, 3H), 3.03 (s, |
| | | | | 3H), 3.01-2.92 (m, 3H), 2.82-2.68 (m, 2H), 2.44- |
| | | | | 2.39 (m, 2H), 2.20 (d, J = 6.4 Hz, 4H), 2.07- |
| | | | | 1.87 (m, 8H), 1.75-1.64 (m, 1H), 1.58 (d, J = 10.8 |
| | | | | Hz, 2H), 1.24-1.12 (m, 2H) |
| I-246^{b} | CAL | ATJ | 788.4 | 11.1 (s, 1H), 10.5 (s, 1H), 8.70 (s, 1H), 8.48-8.44 |
| | | | | (m, 1H), 8.43-8.38 (m, 1H), 8.35 (s, 1H), 8.22 (d, |
| | | | | J = 8.0 Hz, 1H), 7.15 (s, 1H), 7.08-6.92 (m, 3H), |
| | | | | 5.41-5.33 (m, 1H), 4.48-4.37 (m, 1H), 3.99 (s, |
| | | | | 3H), 3.65 (s, 3H), 3.21-3.04 (m, 4H), 3.03-2.84 |
| | | | | (m, 3H), 2.77-2.69 (m, 1H), 2.65-2.59 (m, 1H), |
| | | | | 2.20-2.19 (m, 2H), 2.11 (d, J = 12.8 Hz, 1H), 2.08- |
| | | | | 1.84 (m, 6H), 1.53-1.41 (m, 1H), 1.39-1.33 |
| | | | | (m, 2H), 1.29-1.22 (m, 1H), 1.21-1.13 (m, 3H) |
| I-248^{b} | CAL | BTW | 758.3 | 11.11 (s, 1H), 10.37 (s, 1H), 8.43 (s, 1H), 8.41- |
| | | | | 8.33 (m, 2H), 8.32 (d, J = 0.8 Hz, 1H), 8.18 (dd, J = |
| | | | | 1.2, 7.6 Hz, 1H), 7.65-7.59 (m, 1H), 7.58-7.53 |
| | | | | (m, 1H), 7.10-7.02 (m, 1H), 7.01-6.90 (m, 2H), |
| | | | | 5.38 (dd, J = 5.6, 12.8 Hz, 1H), 4.56-4.44 (m, |
| | | | | 1H), 3.76 (d, J = 11.2 Hz, 1H), 3.65 (s, 3H), 3.62- |
| | | | | 3.53 (m, 1H), 3.22-3.10 (m, 2H), 3.09-2.99 |
| | | | | (m, 2H), 2.99-2.78 (m, 2H), 2.77-2.68 (m, 1H), |
| | | | | 2.66 (s, 1H), 2.31-2.16 (m, 3H), 2.04 (m, 2H), |
| | | | | 2.03-1.96 (m, 3H), 1.94-1.84 (m, 1H), 1.50 (s, 1H), |
| | | | | 1.37 (d, J = 5.6 Hz, 3H), 1.31 (d, J = 12.8 Hz, |
| | | | | 1H), 1.26-1.13 (m, 1H) |
| I-249 | AQK | BWH | 813.5 | 11.10 (s, 1H), 10.53 (s, 1H), 9.32 (d, J = 1.6 Hz, |
| | | | | 1H), 9.25 (d, J = 1.6 Hz, 1H), 8.79-8.75 (m, 1H), |
| | | | | 8.58 (s, 1H), 7.99 (s, 1H), 7.71 (s, 1H), 7.04-6.97 |
| | | | | (m, 1H), 6.96-6.86 (m, 2H), 5.36 (dd, J = 5.2, |
| | | | | 12.8 Hz, 1H), 4.63-4.47 (m, 1H), 3.65 (s, 3H), |
| | | | | 3.27-3.18 (m, 3H), 2.99-2.73 (m, 6H), 2.70- |
| | | | | 2.56 (m, 3H), 2.24-2.16 (m, 2H), 2.12-1.94 (m, |
| | | | | 7H), 1.94-1.61 (m, 4H), 1.40-1.17 (m, 2H) |
| I-252 | CAO | ATJ | 768.3 | 10.55-10.47 (m, 2H), 9.42 (d, J = 11.2 Hz, 1H), |
| | | | | 8.68 (s, 1H), 8.50-8.43 (m, 2H), 8.42-8.36 (m, |
| | | | | 1H), 8.32 (s, 1H), 8.21 (d, J = 7.6 Hz, 1H), 7.62 |
| | | | | (q, J = 7.2 Hz, 1H), 7.39 (s, 1H), 7.14 (s, 1H), 7.09- |
| | | | | 6.97 (m, 1H), 4.70-4.46 (m, 1H), 4.41-4.33 |
| | | | | (m, 1H), 3.98 (s, 3H), 3.95-3.77 (m, 2H), 3.76- |
| | | | | 3.62 (m, 2H), 3.38-3.34 (m, 5H), 2.99-2.91 (m, 1H), |
| | | | | 2.80-2.72 (m, 1H), 2.25-2.08 (m, 3H), 2.04-1.81 |
| | | | | (m, 5H), 1.76-1.50 (m, 1H), 1.28-1.09 (m, 2H) |
| I-253 | CAQ | ATJ | 788.3 | 11.09 (s, 1H), 10.50 (s, 1H), 8.69 (s, 1H), 8.48- |
| | | | | 8.44 (m, 1H), 8.43-8.38 (m, 1H), 8.34 (s, 1H), |
| | | | | 8.24-8.20 (m, 1H), 7.16 (s, 1H), 7.05-6.98 (m, |
| | | | | 2H), 6.97-6.89 (m, 1H), 5.40-5.30 (m, 1H), 4.45- |
| | | | | 4.34 (m, 1H), 4.16-4.08 (m, 2H), 3.98 (s, 3H), |
| | | | | 3.01-2.84 (m, 6H), 2.76-2.68 (m, 1H), 2.67- |
| | | | | 2.56 (m, 2H), 2.28-2.13 (m, 5H), 2.06-1.84 (m, |
| | | | | 6H), 1.75-1.60 (m, 1H), 1.25-1.10 (m, 5H) |
| I-254 | CAO | AGL | 796.4 | 12.36 (s, 1H), 10.51 (d, J = 6.0 Hz, 1H), 9.42 (d, |
| | | | | J = 12.0 Hz, 1H), 8.71 (s, 1H), 8.49-8.42 (m, 2H), |
| | | | | 8.39-8.33 (m, 2H), 8.17-8.13 (m, 1H), 7.63 (q, |
| | | | | J = 7.6 Hz, 1H), 7.56 (s, 1H), 7.44-7.36 (m, 1H), |
| | | | | 7.11-6.98 (m, 1H), 5.94 (s, 1H), 4.58-4.40 (m, |
| | | | | 2H), 4.16-4.02 (m, 1H), 3.99-3.89 (m, 1H), 3.87- |
| | | | | 3.59 (m, 4H), 3.00-2.88 (m, 2H), 2.81-2.72 |
| | | | | (m, 2H), 2.52-2.51 (m, 1H), 2.20-2.09 (m, 4H), |
| | | | | 2.03-1.87 (m, 5H), 1.61 (s, 6H), 1.24-1.13 (m, 2H) |
| I-255 | CAR | ATJ | 788.3 | 11.08 (s, 1H), 10.40 (s, 1H), 8.68 (s, 1H), 8.50- |
| | | | | 8.37 (m, 2H), 8.32 (s, 1H), 8.28-822 (m, 1H), |
| | | | | 7.16 (s, 1H), 7.02-6.92 (m, 2H), 6.88-6.84 (m, |
| | | | | 1H), 5.36 (dd, J = 5.2 Hz, 12.8 1H), 4.45-4.31 |
| | | | | (m, 1H), 3.98 (s, 3H), 3.66 (s, 3H), 3.16 (d, 4H), |
| | | | | 2.94-2.84 (m, 1H), 2.76 (s, 4H), 2.66-2.55 (m, |
| | | | | 2H), 2.32 (d, 2H), 2.16 (d, 2H), 2.05-1.82 (m, |
| | | | | 8H), 1.68-1.51 (m, 1H), 1.21-1.05 (m, 2H) |
| I-256 | CAS | ATJ | 770.4 | 10.54 (s, 1H), 10.51 (s, 1H), 9.77-9.52 (m, 1H), |
| | | | | 8.70 (s, 1H), 8.62-8.51 (m, 1H), 8.48-8.44 (m, |
| | | | | 1H), 8.43-8.38 (m, 1H), 8.34 (s, 1H), 8.23-8.19 |
| | | | | (m, 1H), 7.89-7.69 (m, 2H), 7.62-7.41 (m, 1H), |
| | | | | 7.15 (s, 1H), 4.48-4.37 (m, 1H), 3.99 (s, 3H), |
| | | | | 3.96-3.86 (m, 1H), 3.85-3.59 (m, 3H), 3.59- |
| | | | | 3.47 (m, 1H), 3.21-3.14 (m, 1H), 3.02-2.84 (m, |
| | | | | 2H), 2.83-2.69 (m, 2H), 2.56-2.51 (m, 1H), 2.31- |
| | | | | 2.11 (m, 4H), 2.09-1.89 (m, 5H), 1.38-1.12 (m, |
| | | | | 2H), 0.89 (t, J = 6.8 Hz, 3H) |
| I-257 | CAS | AGL | 798.4 | 12.37 (s, 1H), 10.55 (s, 1H), 9.59 (s, 1H), 8.73 (s, |
| | | | | 1H), 8.55 (d, J = 2.4 Hz, 1H), 8.48-8.44 (m, 1H), |
| | | | | 8.40-8.34 (m, 2H), 8.19-8.13 (m, 1H), 7.83- |
| | | | | 7.77 (m, 1H), 7.75-7.69 (m, 1H), 7.61-7.55 (m, |
| | | | | 1H), 7.48 (s, 1H), 5.95 (s, 1H), 4.53-4.41 (m, |
| | | | | 1H), 4.02-3.85 (m, 1H), 3.78-3.69 (m, 1H), 3.61- |
| | | | | 3.47 (m, 1H), 3.28-3.19 (m, 1H), 3.03-2.84 |
| | | | | (m, 3H), 2.81-2.73 (m, 2H), 2.33-2.24 (m, 2H), |
| | | | | 2.19 (d, J = 11.2 Hz, 2H), 2.09-1.89 (m, 5H), 1.83- |
| | | | | 1.66 (m, 2H), 1.63 (s, 6H), 1.27-1.15 (m, 2H), |
| | | | | 0.90 (t, J = 5.2 Hz, 3H) |
| I-258 | BWC | BVB | 743.4 | 11.11 (s, 1H), 9.91 (s, 1H), 9.31 (s, 1H), 9.21 (d, J = |
| | | | | 2.0 Hz, 1H), 8.78 (s, 1H), 8.31 (s, 1H), 8.08 (s, |
| | | | | 1H), 7.45-7.25 (m, 1H), 7.19-7.05 (m, 2H), 7.03- |
| | | | | 6.94 (m, 1H), 5.38 (dd, J = 4.8, 12.0 Hz, 1H), |
| | | | | 4.48-4.34 (m, 1H), 3.87 (s, 3H), 3.83-3.60 (m, |
| | | | | 4H), 3.57 (s, 3H), 3.18-3.04 (m, 2H), 2.95-2.85 |
| | | | | (m, 1H), 2.79-2.67 (m, 2H), 2.66-2.58 (m, 2H), |
| | | | | 2.21-2.12 (m, 2H), 2.10-1.82 (m, 7H), 1.67- |
| | | | | 1.47 (m, 1H), 1.34-1.18 (m, 2H) |
| I-259 | BXL | BVB | 713.6 | 10.53 (s, 1H), 9.90 (s, 1H), 9.43 (s, 1H), 9.34- |
| | | | | 9.30 (m, 1H), 9.22-9.18 (m, 1H), 8.78 (s, 1H), |
| | | | | 8.53 (s, 1H), 8.32 (s, 1H), 8.08 (s, 1H), 7.76-7.69 |
| | | | | (m, 1H), 7.62 (d, J = 8.4 Hz, 1H), 7.29 (d, J = 7.2 |
| | | | | Hz, 1H), 7.09 (s, 1H), 4.45-4.33 (m, 1H), 3.95- |
| | | | | 3.89 (m, 1H), 3.88 (s, 3H), 3.74-3.67 (m, 1H), |
| | | | | 3.48-3.40 (m, 1H), 3.24-3.09 (m, 4H), 3.03- |
| | | | | 2.90 (m, 1H), 2.79-2.67 (m, 4H), 2.36-2.28 (m, |
| | | | | 2H), 2.21-2.12 (m, 2H), 2.06-1.97 (m, 2H), 1.97- |
| | | | | 1.86 (m, 2H), 1.79-1.64 (m, 1H), 1.25-1.09 (m, 2H) |
| I-260 | BAI | BSC | 758.5 | 11.10 (s, 1H), 10.14 (s, 1H), 8.51-8.31 (m, 3H), |
| | | | | 8.24-8.11 (m, 2H), 7.52 (s, 1H), 7.09-6.85 (m, |
| | | | | 3H), 5.37 (d, J = 5.2 Hz, 1H), 4.53-4.39 (m, 1H), |
| | | | | 3.65 (s, 3H), 3.15-2.82 (m, 6H), 2.77-2.58 (m, |
| | | | | 4H), 2.41 (s, 3H), 2.30-2.12 (m, 4H), 2.08-1.86 |
| | | | | (m, 6H), 1.84-1.57 (m, 1H), 1.30-1.08 (m, 2H) |
| I-261^{b} | BXL | BSC | 740.3 | 10.5 (s, 1H), 10.13 (s, 1H), 9.54-9.38 (m, 1H), |
| | | | | 8.56 (s, 1H), 8.46-8.35 (m, 3H), 8.23-8.12 (m, |
| | | | | 2H), 7.79-7.71 (m, 1H), 7.70-7.62 (m, 1H), 7.52 |
| | | | | (s, 1H), 7.37-7.23 (m, 1H), 4.53-4.40 (m, 1H), |
| | | | | 3.96-3.87 (m, 1H), 3.73-3.74 (m, 1H), 3.29- |
| | | | | 3.11 (m, 5H), 2.97-2.93 (m, 2H), 2.79-2.72 (m, |
| | | | | 2H), 2.52 (s, 1H), 2.41 (s, 3H), 2.20 (d, J = 10.0 |
| | | | | Hz, 3H), 2.11-1.88 (m, 5H), 1.85-1.67 (m, 1H), |
| | | | | 1.32-1.14 (m, 2H) |
| I-262^{b} | BWS | BVB | 757.4 | 11.13-11.03 (m, 1H), 9.91 (s, 1H), 9.31 (s, 1H), |
| | | | | 9.20 (d, J = 1.6 Hz, 1H), 8.78 (s, 1H), 8.32 (s, 1H), |
| | | | | 8.08 (s, 1H), 7.13-7.07 (m, 2H), 7.00 (d, J = 7.8 |
| | | | | Hz, 1H), 6.91 (d, J = 7.6 Hz, 1H), 5.35 (d, J = 12.6 |
| | | | | Hz, 1H), 4.45-4.34 (m, 1H), 3.88 (s, 3H), 3.71 (s, |
| | | | | 3H), 3.07 (d, J = 10.4 Hz, 2H), 2.80 (d, J = 7.8 Hz, |
| | | | | 2H), 2.70 (d, J = 5.6 Hz, 1H), 2.65-2.62 (m, 1H), |
| | | | | 2.60 (br d, J = 0.8 Hz, 1H), 2.26 (d, J = 6.8 Hz, |
| | | | | 3H), 2.20-2.13 (m, 3H), 2.07 (d, J = 12 Hz, 2H), |
| | | | | 1.90 (d, J = 5.2 Hz, 6H), 1.60-1.50 (m, 1H), 1.23- |
| | | | | 1.14 (m, 2H) |
| I-263 | CAT | AGL | 886.5 | 11.10 (s, 1H), 8.72 (s, 1H), 8.46-8.43 (m, 1H), |
| | | | | 8.40-8.34 (m, 2H), 8.18-8.14 (m, 1H), 7.62 (t, J = |
| | | | | 7.6 Hz, 1H), 7.55 (s, 1H), 7.12 (d, J = 6.0 Hz, |
| | | | | 1H), 6.90 (d, J = 8.8 Hz, 1H), 6.69-6.54 (m, 1H), |
| | | | | 5.94 (s, 1H), 5.07 (dd, J = 5.6, 12.8 Hz, 1H), 4.51- |
| | | | | 4.40 (m, 1H), 4.14-4.05 (m, 1H), 3.80-3.49 (m, |
| | | | | 3H), 2.95-2.80 (m, 4H), 2.64-2.60 (m, 2H), 2.57 |
| | | | | (s, 3H), 2.55-2.53 (m, 1H), 2.37 (s, 3H), 2.25-2.14 |
| | | | | (m, 4H), 2.07-2.00 (m, 2H), 1.99-1.92 (m, 4H), |
| | | | | 1.91-1.68 (m, 4H), 1.62 (s, 6H), 1.28-1.17 (m, 2H) |
| I-264 | AOQ | CAU | 801.8^{e} | 12.38 (s, 1H), 11.09 (s, 1H), 8.69 (s, 1H), 8.32 (s, |
| | | | | 1H), 8.01-7.87 (m, 2H), 7.64-7.53 (m, 2H), 7.52- |
| | | | | 7.45 (m, 1H), 7.07 (d, J = 7.2 Hz, 1H), 6.92 (d, J = |
| | | | | 8.4 Hz, 1H), 6.51 (d, J = 6.0 Hz, 1H), 5.90 (s, |
| | | | | 1H), 5.07-5.03 (m, 1H), 4.49-4.33 (m, 1H), 4.22- |
| | | | | 4.05 (m, 2H), 3.37-3.35 (m, 3H), 2.94-2.80 |
| | | | | (m, 1H), 2.60 (s, 3H), 2.56 (s, 1H), 2.52-2.51 (m, |
| | | | | 2H), 2.42-2.35 (m, 3H), 2.28-2.20 (m, 7H), 2.14- |
| | | | | 2.12 (m, 2H), 2.06-2.00 (m, 1H), 1.95-1.90 (m, 4H), |
| | | | | 1.72-1.66 (m, 2H), 1.63 (s, 6H), 1.17-1.06 (m, 2H) |
| I-265 | AOQ | CAW | 805.8 | 12.32 (s, 1H), 11.09 (s, 1H), 8.74-8.69 (m, 1H), |
| | | | | 8.67 (s, 1H), 8.33 (s, 1H), 8.19 (d, J = 6.4 Hz, 1H), |
| | | | | 8.06 (t, J = 1., 7.6 Hz, 1H), 7.65-7.62 (m, 1H), |
| | | | | 7.58 (d, J = 7.2, 8.4 Hz, 1H), 7.54 (s, 1H), 7.07 (d, |
| | | | | J = 6.8 Hz, 1H), 6.92 (d, J = 8.4 Hz, 1H), 6.50 (d, |
| | | | | J = 6.0 Hz, 1H), 6.02 (s, 1H), 5.07-5.02 (m, 1H), |
| | | | | 4.46-4.36 (m, 1H), 4.19-4.09 (m, 2H), 3.35 (s, |
| | | | | 2H), 2.92-2.83 (m, 1H), 2.61-2.55 (m, 1H), 2.52- |
| | | | | 2.51 (m, 1H), 2.43-2.38 (m, 2H), 2.37-2.34 |
| | | | | (m, 2H), 2.27-2.20 (m, 2H), 2.16 (s, 4H), 2.15- |
| | | | | 2.11 (m, 3H), 2.05-1.99 (m, 1H), 1.96-1.86 (m, 4H), |
| | | | | 1.69-1.63 (m, 2H), 1.61 (s, 6H), 1.15-1.02 (m, 3H) |
| I-266 | CAX | ATJ | 757.3 | 11.14 (s, 1H), 10.51 (s, 2H), 8.70 (s, 1H), 8.54- |
| | | | | 8.31 (m, 3H), 8.22 (d, J = 7.6 Hz, 1H), 7.22-7.14 |
| | | | | (m, 2H), 7.11 (t, J = 7.6 Hz, 1H), 7.00 (d, J = 7.6 |
| | | | | Hz, 1H), 6.04 (s, 1H), 5.44 (d, J = 5.2, 12.7 Hz, |
| | | | | 1H), 4.83-4.11 (m, 5H), 3.99 (s, 3H), 3.37 (s, 3H), |
| | | | | 3.00-2.86 (m, 1H), 2.82-2.70 (m, 1H), 2.69-2.63 (m, |
| | | | | 1H), 2.59-2.52 (m, 2H), 2.20 (d, J = 10.4 Hz, |
| | | | | 2H), 2.10-1.89 (m, 6H), 1.30 (d, J = 11.6 Hz, 2H) |
| I-267 | CAY | CAZ | 751.1 | 11.1 (s, 1H), 8.58-8.54 (m, 2H), 8.47 (d, J = 4.0 |
| | | | | Hz, 1H), 8.42-8.39 (m, 1H), 8.31 (d, J = 7.6 Hz, |
| | | | | 1H), 8.20 (s, 1H), 8.03 (dd, J = 2.8, 9.2 Hz, 1H), |
| | | | | 6.97 (d, J = 8.4 Hz, 1H), 6.88 (s, 1H), 6.75 (d, J = |
| | | | | 4.0 Hz, 1H), 6.66 (d, J = 7.4 Hz, 1H), 5.30 (dd, J = |
| | | | | 5.2, 12.8 Hz, 1H), 3.82-3.76 (m, 1H), 3.74 (d, |
| | | | | J = 6.0 Hz, 1H), 3.32 (s, 3H), 3.16-3.04 (m, 3H), |
| | | | | 2.97-2.83 (m, 2H), 2.76-2.65 (m, 2H), 2.61 (d, |
| | | | | J = 16.4 Hz, 2H), 2.53 (d, J = 6.8 Hz, 3H), 2.02- |
| | | | | 1.96 (m, 1H), 1.93-1.83 (m, 4H), 1.70-1.51 (m, |
| | | | | 1H), 1.49-1.31 (m, 3H), 1.29 (d, J = 6.4 Hz, 6H), |
| | | | | 1.16-0.96 (m, 2H) |
| I-268 | CBA | ATJ | 842.4 | 11.1 (s, 1H), 10.50 (s, 1H), 8.69 (s, 1H), 8.47- |
| | | | | 8.43 (m, 1H), 8.42-8.37 (m, 1H), 8.32 (s, 1H), |
| | | | | 8.21 (d, J = 7.6 Hz, 1H), 7.15 (s, 1H), 7.10-7.06 |
| | | | | (m, 1H), 7.06-7.02 (m, 1H), 7.01-6.97 (m, 1H), |
| | | | | 5.36 (dd, J = 5.6, 12.4 Hz, 1H), 4.42-4.32 (m, |
| | | | | 1H), 3.98 (s, 3H), 3.57 (s, 3H), 3.13-3.03 (m, |
| | | | | 1H), 3.00-2.82 (m, 4H), 2.81-2.66 (m, 4H), 2.66-2.54 |
| | | | | (m, 4H), 2.54 (s, 3H), 2.14 (d, J = 10.4 Hz, 4H), |
| | | | | 2.03-1.82 (m, 8H), 1.65 (s, 1H), 1.20-1.08 (m, 2H) |
| I-269 | BSN | BSC | 739.4 | 10.55 (s, 1H), 10.14 (s, 1H), 9.67 (s, 1H), 8.64- |
| | | | | 8.56 (m, 1H), 8.49-8.32 (m, 3H), 8.22 (d, J = 3.2 |
| | | | | Hz, 2H), 8.00-7.74 (m, 2H), 7.70-7.47 (m, 2H), |
| | | | | 4.56-4.40 (m, 1H), 3.94 (d, J = 5.2, 9.9, 12.1 Hz, |
| | | | | 1H), 3.88-3.66 (m, 2H), 3.52-3.37 (m, 1H), 2.99 |
| | | | | (d, J = 16.4 Hz, 2H), 2.89-2.58 (m, 4H), 2.42 (s, |
| | | | | 3H), 2.21 (d, J = 10.8 Hz, 2H), 2.13-1.80 (m, |
| | | | | 9H), 1.30-1.19 (m, 2H) |
| I-271 | BRY | BXI | 750.6 | 10.64 (s, 1H), 10.56-10.50 (m, 1H), 9.28-9.23 |
| | | | | (m, 1H), 8.66-8.61 (m, 1H), 8.48 (s, 1H), 8.47- |
| | | | | 8.36 (m, 3H), 8.26-8.19 (m, 2H), 8.07 (s, 1H), |
| | | | | 7.92 (d, J = 8.8 Hz, 1H), 7.79 (dd, J = 1.2, 8.8 Hz, |
| | | | | 1H), 4.63-4.52 (m, 1H), 3.99-3.87 (m, 1H), 3.76- |
| | | | | 3.66 (m, 1H), 3.07-2.92 (m, 3H), 2.76 (td, J = |
| | | | | 5.6, 16.8 Hz, 2H), 2.26-2.16 (m, 4H), 2.11-1.94 |
| | | | | (m, 6H), 1.89-1.63 (m, 5H), 1.23-1.11 (m, 2H) |
| I-272 | CCK | ATJ | 770.2 | 10.52 (d, J = 13.6 Hz, 2H), 9.52 (s, 1H), 8.70 (s, |
| | | | | 1H), 8.55 (s, 1H), 8.48-8.37 (m, 2H), 8.35 (s, |
| | | | | 1H), 8.22 (dd, J = 0.8, 7.6 Hz, 1H), 7.78-7.70 (m, |
| | | | | 1H), 7.64 (d, J = 8.4 Hz, 1H), 7.37 (d, J = 7.2 Hz, |
| | | | | 1H), 7.15 (s, 1H), 4.48-4.37 (m, 1H), 3.99 (s, |
| | | | | 3H), 3.91 (m, 1H), 3.70 (m, 1H), 3.60 (s, 2H), 3.42 |
| | | | | (m, 5H), 3.09 (s, 1H), 2.97 (m, 2H), 2.76 (m, 1H), |
| | | | | 2.52 (d, J = 2.0 Hz, 1H), 2.20 (d, J = 9.6 Hz, 4H), |
| | | | | 2.07-1.85 (m, 5H), 1.36-1.21 (m, 2H) |
| I-273 | CCL | AGL | 798.3 | 12.37 (s, 1H), 10.55 (s, 1H), 9.62-9.42 (m, 1H), |
| | | | | 8.73 (s, 1H), 8.63-8.52 (m, 1H), 8.48-8.33 (m, |
| | | | | 3H), 8.21-8.11 (m, 1H), 7.75 (d, J = 2.8 Hz, 2H), |
| | | | | 7.57 (s, 1H), 7.44-7.25 (m, 1H), 5.95 (s, 1H), |
| | | | | 4.55-4.40 (m, 1H), 3.99-3.81 (m, 2H), 3.74- |
| | | | | 3.51 (m, 3H), 3.17-3.04 (m, 2H), 3.03-2.87 (m, |
| | | | | 2H), 2.84-2.73 (m, 2H), 2.28-2.09 (m, 4H), 2.07- |
| | | | | 1.88 (m, 4H), 1.62 (s, 7H), 1.50-1.29 (m, 3H), |
| | | | | 1.25-1.07 (m, 2H) |
| I-276 | CBX | BRR | 772.3 | 11.1 (s, 1H), 10.20 (s, 1H), 9.07 (s, 1H), 8.61 (s, |
| | | | | 1H), 8.51-8.49 (m, 1H), 8.47 (s, 1H), 8.41 (t, J = |
| | | | | 7.6 Hz, 1H), 8.23 (dd, J = 0.8, 7.6 Hz, 1H), 7.03- |
| | | | | 6.93 (m, 1H), 6.75-6.63 (m, 1H), 5.66 (s, 1H), |
| | | | | 5.35 (dd, J = 5.6, 12.4 Hz, 1H), 4.67-4.57 (m, |
| | | | | 1H), 3.75 (s, 3H), 3.34-3.34 (m, 3H), 2.96-2.81 |
| | | | | (m, 2H), 2.76-2.57 (m, 4H), 2.55-2.51 (m, 1H), |
| | | | | 2.48-2.42 (m, 3H), 2.24-2.22 (m, 3H), 2.06- |
| | | | | 1.96 (m, 6H), 1.35-1.21 (m, 2H) |
| I-277 | CBX | ATJ | 801.3 | 11.10 (s, 1H), 10.51 (s, 1H), 8.70 (s, 1H), 8.49- |
| | | | | 8.44 (m, 1H), 8.44-8.38 (m, 1H), 8.35 (s, 1H), |
| | | | | 8.22 (dd, J = 0.8, 7.6 Hz, 1H), 7.16 (s, 1H), 6.99 |
| | | | | (d, J = 7.6 Hz, 1H), 6.71 (d, J = 8.0 Hz, 1H), 5.64 |
| | | | | (s, 1H), 5.35 (dd, J = 4.4, 12.4 Hz, 1H), 4.46-4.35 |
| | | | | (m, 1H), 3.99 (s, 3H), 3.74 (s, 3H), 3.38-3.33 (m, |
| | | | | 4H), 2.95-2.84 (m, 1H), 2.76-2.68 (m, 1H), 2.67- |
| | | | | 2.56 (m, 2H), 2.56-2.51 (m, 7H), 2.18 (d, J = |
| | | | | 10.8 Hz, 2H), 2.05-1.89 (m, 5H), 1.35-1.12 (m, 2H) |
| I-278 | AZK | CAZ | 750.4 | 11.1 (s, 1H), 8.59-8.54 (m, 2H), 8.47 (d, J = 3.6 |
| | | | | Hz, 1H), 8.42-8.39 (m, 1H), 8.29 (d, J = 7.6 Hz, |
| | | | | 1H), 8.19 (s, 1H), 8.03 (dd, J = 2.8, 9.2 Hz, 1H), |
| | | | | 7.02 (d, J = 4.8 Hz, 2H), 6.99-6.95 (m, 1H), 6.75 |
| | | | | (d, J = 4.0 Hz, 1H), 5.37 (dd, J = 5.2, 12.4 Hz, |
| | | | | 1H), 3.81-3.71 (m, 2H), 3.59 (s, 3H), 3.02 (d, J = |
| | | | | 10.4 Hz, 2H), 2.93-2.85 (m, 1H), 2.77-2.68 |
| | | | | (m, 1H), 2.67-2.55 (m, 2H), 2.24 (s, 2H), 2.15 (s, |
| | | | | 2H), 1.99-1.93 (m, 1H), 1.88-1.83 (m, 4H), 1.80 |
| | | | | (s, 4H), 1.57-1.48 (m, 1H), 1.42-1.32 (m, 2H), |
| | | | | 1.29 (d, J = 6.4 Hz, 6H), 1.03-0.92 (m, 2H) |
| I-279 | BWS | BRR | 771.7 | 11.1 (s, 1H), 10.2 (s, 1H), 9.07 (s, 1H), 8.63 (s, |
| | | | | 1H), 8.51 (d, J = 1.2 Hz, 1H), 8.50-8.47 (m, 1H), |
| | | | | 8.44-8.39 (m, 1H), 8.24 (d, J = 7.6 Hz, 1H), 7.20- |
| | | | | 6.95 (m, 3H), 5.38 (dd, J = 5.2, 12.4 Hz, 1H), |
| | | | | 4.69-4.57 (m, 1H), 4.26-4.06 (m, 1H), 3.71 (s, |
| | | | | 3H), 3.63-3.37 (m, 2H), 3.17-2.96 (m, 3H), 2.96- |
| | | | | 2.82 (m, 2H), 2.76-2.67 (m, 1H), 2.66-2.59 |
| | | | | (m, 1H), 2.52 (d, J = 2.0 Hz, 1H), 2.25 (d, J = 10.8 |
| | | | | Hz, 5H), 2.09 (d, J = 13.6 Hz, 3H), 2.05-1.95 (m, |
| | | | | 4H), 1.39-1.24 (m, 2H) |
| I-280 | BXY | BVB | 739.3 | 10.54 (s, 1H), 9.91 (s, 1H), 9.54 (s, 1H), 9.31 (d, |
| | | | | J = 1.2 Hz, 1H), 9.21 (d, J = 1.6 Hz, 1H), 8.78 (s, |
| | | | | 1H), 8.56 (s, 1H), 8.33 (s, 1H), 8.09 (s, 1H), 7.82- |
| | | | | 7.57 (m, 2H), 7.37 (d, J = 1.6 Hz, 1H), 7.08 (s, |
| | | | | 1H), 4.43 (t, J = 11.6 Hz, 1H), 3.97-3.90 (m, 1H), |
| | | | | 3.88 (s, 3H), 3.70 (td, J = 6.0, 12.0 Hz, 1H), 3.32- |
| | | | | 3.29 (m, 3H), 3.26-3.05 (m, 3H), 3.01-2.92 (m, 1H), |
| | | | | 2.76 (td, J = 5.6, 16.4 Hz, 1H), 2.59 (s, 2H), 2.19 |
| | | | | (d, J = 10.4 Hz, 4H), 2.08 (d, J = 12.0 Hz, 3H), |
| | | | | 2.02-1.85 (m, 3H), 1.85-1.50 (m, 1H), 1.36-1.12 (m, 2H) |
| I-282 | BXL | CCR | 774.3 | 10.53 (s, 1H), 10.34 (s, 1H), 9.43 (s, 1H), 8.72 (s, |
| | | | | 1H), 8.53 (s, 1H), 8.47-8.33 (m, 2H), 8.21 (d, J = |
| | | | | 7.6 Hz, 1H), 7.76-7.70 (m, 1H), 7.62 (d, J = 8.4 |
| | | | | Hz, 1H), 7.29 (t, J = 3.6 Hz, 2H), 4.03 (d, J = 12.0 |
| | | | | Hz, 2H), 3.96 (s, 3H), 3.93-3.86 (m, 1H), 3.70 (td, |
| | | | | J = 6.0, 12.4 Hz, 1H), 3.23-3.08 (m, 6H), 2.98-2.94 |
| | | | | (m, 1H), 2.79-2.74 (m, 1H), 2.70 (s, 4H), 2.34-2.31 |
| | | | | (m, 2H), 1.89 (d, J = 11.6 Hz, 3H), 1.31-1.18 (m, 2H) |
| I-283 | CCS | BRR | 775.3 | 11.07 (s, 1H), 10.20 (s, 1H), 9.07 (s, 1H), 8.61 (s, |
| | | | | 1H), 8.52-8.45 (m, 2H), 8.41 (t, J = 7.6 Hz, 1H), |
| | | | | 8.23 (d, J = 7.6 Hz, 1H), 6.88 (d, J = 8.4 Hz, 1H), |
| | | | | 6.68 (d, J = 8.8 Hz, 1H), 5.36-5.26 (m, 1H), 4.65- |
| | | | | 4.53 (m, 1H), 3.79 (s, 3H), 3.62 (s, 3H), 3.51 (t, |
| | | | | J = 10.0 Hz, 2H), 2.83-2.74 (m, 4H), 2.65-2.57 |
| | | | | (m, 3H), 2.25-2.14 (m, 6H), 1.98 (t, J = 11.2 Hz, |
| | | | | 5H), 1.70 (s, 1H), 1.25-1.07 (m, 2H) |
| I-284 | BXL | BKQ | 744.3 | 10.53 (s, 1H), 10.24 (d, J = 2.4 Hz, 1H), 9.44 (s, |
| | | | | 1H), 8.54 (s, 1H), 8.51 (s, 1H), 8.48-8.37 (m, |
| | | | | 3H), 8.24-8.20 (m, 1H), 7.76-7.69 (m, 1H), 7.62 |
| | | | | (d, J = 8.4 Hz, 1H), 7.55 (d, J = 12.0 Hz, 1H), 7.29 |
| | | | | (d, J = 7.6 Hz, 1H), 4.52-4.43 (m, 1H), 3.91 (m, |
| | | | | J = 5.2, 9.6, 12.0 Hz, 1H), 3.71 (m, J = 6.0, 12.0 |
| | | | | Hz, 1H), 3.16 (d, J = 5.2 Hz, 4H), 2.97 (m, J = 6.0, |
| | | | | 10.0, 16.4 Hz, 1H), 2.80-2.67 (m, 5H), 2.32 (d, J = |
| | | | | 7.2 Hz, 2H), 2.18 (d, J = 11.2 Hz, 2H), 2.05- |
| | | | | 1.89 (m, 4H), 1.78-1.67 (m, 1H), 1.24-1.11 (m, 2H) |
| I-285 | CCY | CCX | 824.4 | 10.56 (d, J = 2.4 Hz, 1H), 10.51 (s, 1H), 9.42 (d, |
| | | | | J = 8.4 Hz, 1H), 8.70 (s, 1H), 8.61 (d, J = 3.2 Hz, |
| | | | | 1H), 8.49-8.44 (m, 1H), 8.44-8.38 (m, 1H), 8.23 |
| | | | | (d, J = 7.6 Hz, 1H), 7.93-7.88 (m, 1H), 7.87- |
| | | | | 7.81 (m, 1H), 7.71 (d, J = 7.2 Hz, 1H), 7.28-6.98 |
| | | | | (m, 2H), 4.60-4.19 (m, 2H), 3.98 (s, 3H), 3.95- |
| | | | | 3.89 (m, 1H), 3.72 (d, J = 2.8 Hz, 2H), 3.05-2.92 |
| | | | | (m, 2H), 2.88-2.69 (m, 4H), 2.52 (d, J = 2.0 Hz, |
| | | | | 10H), 2.23-1.86 (m, 8H), 1.41-1.06 (m, 3H) |
| I-286 | CCY | CCZ | 824.0 | 10.51 (d, J = 12 Hz, 2H), 9.39 (s, 1H), 8.68 (s, |
| | | | | 1H), 8.56 (s, 1H), 8.49-8.44 (m, 1H), 8.43-8.36 |
| | | | | (m, 1H), 8.33 (s, 1H), 8.22 (d, J = 7.6 Hz, 1H), |
| | | | | 7.87-7.71 (m, 2H), 7.53 (d, J = 7.2 Hz, 1H), 7.16 |
| | | | | (s, 1H), 4.42-4.30 (m, 1H), 4.22-4.10 (m, 1H), |
| | | | | 3.98 (s, 3H), 3.95-3.86 (m, 1H), 3.78-3.64 (m, |
| | | | | 1H), 3.04-2.91 (m, 1H), 2.84-2.68 (m, 3H), 2.64- |
| | | | | 2.56 (m, 2H), 2.36 (d, J = 6 Hz, 7H), 2.14 (d, J = |
| | | | | 6 Hz, 5H), 1.98-1.85 (m, 6H), 1.69-1.52 (m, |
| | | | | 1H), 1.31-0.99 (m, 3H) |
| I-287 | BAI | CEE | 736.6 | 11.08 (s, 1H), 9.65 (s, 1H), 8.72 (s, 1H), 8.47 (d, J = |
| | | | | 2.8 Hz, 1H), 8.29 (s, 1H), 8.02 (s, 1H), 7.85 (s, |
| | | | | 1H), 7.07 (s, 1H), 7.02-6.88 (m, 3H), 5.37-5.33 |
| | | | | (m, 1H), 4.43-4.34 (m, 1H), 3.91 (s, 3H), 3.87 (s, |
| | | | | 3H), 3.63 (s, 3H), 3.01-2.84 (m, 7H), 2.75-2.59 |
| | | | | (m, 3H), 2.31-2.26 (m, 1H), 2.21-2.12 (m, 3H), |
| | | | | 2.05-1.87 (m, 6H), 1.74-1.67 (m, 1H), 1.21-1.12 (m, 2H) |
| I-288 | BXL | BXI | 751.4 | 10.64 (s, 1H), 10.53 (s, 1H), 9.43 (s, 1H), 8.67- |
| | | | | 8.61 (m, 1H), 8.53 (s, 1H), 8.47-8.37 (m, 3H), |
| | | | | 8.28-8.20 (m, 2H), 7.77-7.68 (m, 1H), 7.62 (d, |
| | | | | J = 8.0 Hz, 1H), 7.29 (d, J = 7.2 Hz, 1H), 4.66- |
| | | | | 4.51 (m, 1H), 3.95-3.86 (m, 1H), 3.75-3.66 (m, |
| | | | | 1H), 3.20-3.10 (m, 4H), 2.98-2.94 (m, 1H), 2.80- |
| | | | | 2.75 (m, 1H), 2.74-2.69 (m, 3H), 2.24-2.17 |
| | | | | (m, 3H), 2.13 (s, 1H), 2.07-1.93 (m, 5H), 1.80- |
| | | | | 1.69 (m, 1H), 1.24-1.14 (m, 2H) |
| I-289 | CCK | BVB | 727.6 | 10.5 (s, 1H), 9.90 (s, 1H), 9.49 (s, 1H), 9.31 (d, J = |
| | | | | 1.6 Hz, 1H), 9.20 (d, J = 2.0 Hz, 1H), 8.78 (s, |
| | | | | 1H), 8.52 (s, 1H), 8.32 (s, 1H), 8.09 (s, 1H), 7.74- |
| | | | | 7.67 (m, 1H), 7.58 (d, J = 8.4 Hz, 1H), 7.33 (d, J = |
| | | | | 7.6 Hz, 1H), 7.08 (s, 1H), 4.47-4.35 (m, 1H), |
| | | | | 3.96-3.89 (m, 1H), 3.88 (s, 3H), 3.72-3.66 (m, |
| | | | | 1H), 3.54 (s, 2H), 3.48-3.39 (m, 3H), 3.26-3.14 |
| | | | | (m, 3H), 3.11-2.92 (m, 2H), 2.79-2.72 (m, 2H), |
| | | | | 2.19-2.16 (m, 2H), 2.12 (s, 2H), 2.03-1.96 (m, 2H), |
| | | | | 1.96-1.93 (m, 2H), 1.78-1.76 (m, 1H), 1.27-1.14 (m, 2H) |
| I-290 | BAI | CAZ | 751.4 | 11.1 (s, 1H), 8.58-8.54 (m, 2H), 8.47 (d, J = 4.0 |
| | | | | Hz, 1H), 8.41 (dd, J = 1.2, 2.8 Hz, 1H), 8.31 (d, J = |
| | | | | 7.4 Hz, 1H), 8.20 (s, 1H), 8.03 (dd, J = 2.8, 9.4 |
| | | | | Hz, 1H), 7.00 (d, J = 7.4 Hz, 1H), 6.94 (d, J = 8.0 |
| | | | | Hz, 2H), 6.75 (d, J = 4.0 Hz, 1H), 5.36 (dd, J = |
| | | | | 5.6, 12.4 Hz, 1H), 3.83-3.76 (m, 1H), 3.76-3.70 |
| | | | | (m, 1H), 3.63 (s, 3H), 3.17-3.04 (m, 2H), 3.00- |
| | | | | 2.81 (m, 4H), 2.76-2.70 (m, 1H), 2.70-2.66 (m, |
| | | | | 1H), 2.64 (s, 1H), 2.60 (s, 1H), 2.54 (s, 1H), 2.24- |
| | | | | 2.22 (m, 2H), 1.99-1.96 (m, 1H), 1.93-1.84 |
| | | | | (m, 4H), 1.62-1.44 (m, 1H), 1.37 (d, J = 11.6 Hz, |
| | | | | 2H), 1.29 (d, J = 6.4 Hz, 6H), 1.11-0.91 (m, 2H) |
| I-291 | CEH | ATJ | 741.1 | 10.61-10.47 (m, 2H), 9.75-9.59 (m, 1H), 8.69 |
| | | | | (s, 1H), 8.62 (s, 1H), 8.49-8.37 (m, 2H), 8.34 (s, |
| | | | | 1H), 8.22 (d, J = 1.2, 7.6 Hz, 1H), 7.98-7.89 (m, |
| | | | | 1H), 7.87-7.75 (m, 2H), 7.14 (s, 1H), 4.45-4.38 |
| | | | | (m, 1H), 3.98 (s, 3H), 3.97-3.83 (m, 2H), 3.75- |
| | | | | 3.68 (m, 1H), 2.99 (d, J = 6.0, 16.4 Hz, 2H), 2.81- |
| | | | | 2.74 (m, 1H), 2.64-2.51 (m, 6H), 2.18 (d, J = |
| | | | | 10.4 Hz, 2H), 2.08-1.87 (m, 5H), 1.79-1.62 (m, |
| | | | | 1H), 1.35-1.17 (m, 2H) |
| I-293 | BSN | ALU | 775.2 | 10.55 (s, 1H), 10.50 (s, 1H), 9.68 (s, 1H), 8.59 (d, |
| | | | | J = 7.2 Hz, 2H), 8.52-8.36 (m, 3H), 8.23 (d, J = |
| | | | | 7.6 Hz, 1H), 8.00 (s, 1H), 7.97-7.88 (m, 1H), 7.82 |
| | | | | (t, J = 7.6 Hz, 1H), 7.67-7.55 (m, 1H), 7.41-7.08 |
| | | | | (m, 1H), 4.66-4.49 (m, 1H), 4.09-3.84 (m, 2H), |
| | | | | 3.71 (td, J = 6.0, 12.0 Hz, 2H), 3.49-3.37 (m, |
| | | | | 2H), 3.01-2.97 (m, 2H), 2.77 (td, J = 5.2, 16.8 |
| | | | | Hz, 2H), 2.23 (d, J = 9.2 Hz, 3H), 2.15-1.65 (m, |
| | | | | 9H), 1.38-1.19 (m, 2H) |
| I-294 | BAI | CEM | 788.3 | 11.09 (s, 1H), 10.73 (s, 1H), 8.70 (s, 1H), 8.49- |
| | | | | 8.36 (m, 2H), 8.33 (s, 1H), 8.21 (dd, J = 0.8, 7.6 |
| | | | | Hz, 1H), 7.13 (s, 1H), 7.02-6.91 (m, 2H), 6.89 (d, |
| | | | | J = 7.2 Hz, 1H), 5.35 (dd, J = 5.2, 12.4 Hz, 1H), |
| | | | | 4.43-4.32 (m, 1H), 4.20-4.18 (m, 2H), 3.63 (s, |
| | | | | 3H), 3.13-2.77 (m, 7H), 2.69-2.57 (m, 2H), 2.54- |
| | | | | 2.52 (m, 1H), 2.24 (d, J = 7.2 Hz, 3H), 2.15 (d, J = |
| | | | | 11.6 Hz, 2H), 2.04-1.86 (m, 5H), 1.71-1.61 |
| | | | | (m, 1H), 1.50 (t, J = 6.8 Hz, 3H), 1.19-1.08 (m, 2H) |
| I-295 | CCS | BWF | 812.3 | 11.08 (s, 1H), 9.13 (s, 1H), 8.88-8.76 (m, 2H), |
| | | | | 8.67 (d, J = 2.0 Hz, 1H), 8.57 (s, 1H), 8.52 (d, J = |
| | | | | 3.6 Hz, 1H), 8.32 (d, J = 6.8 Hz, 1H), 6.95 (d, J = |
| | | | | 8.4 Hz, 1H), 6.88 (d, J = 4.0 Hz, 1H), 6.74 (d, J = |
| | | | | 8.8 Hz, 1H), 5.33 (dd, J = 5.6, 12.4 Hz, 1H), 4.69- |
| | | | | 4.51 (m, 1H), 3.94-3.86 (m, 1H), 3.83 (s, 3H), |
| | | | | 3.75 (t, J = 12.4 Hz, 2H), 3.62 (s, 3H), 3.59-3.52 |
| | | | | (m, 2H), 3.24-3.19 (m, 1H), 3.18-3.04 (m, 5H), |
| | | | | 2.95-2.82 (m, 1H), 2.75-2.68 (m, 1H), 2.64- |
| | | | | 2.59 (m, 1H), 2.31-2.24 (m, 2H), 2.08-1.87 (m, |
| | | | | 6H), 1.36 (d, J = 6.4 Hz, 6H), 1.21-1.13 (m, 2H) |
| I-296 | BAI | CEN | 784.3 | 11.10 (s, 1H), 10.75 (s, 1H), 8.65 (s, 1H), 8.51- |
| | | | | 8.46 (m, 1H), 8.45-8.37 (m, 2H), 8.23 (d, J = 7.6 |
| | | | | Hz, 1H), 7.48 (s, 1H), 7.07-6.98 (m, 1H), 6.95 (d, |
| | | | | J = 7.2 Hz, 2H), 5.37 (dd, J = 4.8, 12.8 Hz, 1H), |
| | | | | 4.53-4.37 (m, 1H), 3.64 (s, 3H), 3.25-3.12 (m, 3H), |
| | | | | 3.06-2.80 (m, 4H), 2.79-2.62 (m, 3H), 2.25-2.11 (m, |
| | | | | 3H), 2.00 (dd, J = 7.2, 12.6 Hz, 8H), 1.36-1.19 |
| | | | | (m, 2H), 1.11 (d, J = 8.4 Hz, 3H), 0.85-0.70 (m, 2H) |
| I-297 | BAI | CEO | 775.3 | 11.10 (s, 1H), 10.24 (s, 1H), 9.62 (s, 1H), 9.50 (s, |
| | | | | 1H), 8.66 (s, 1H), 8.36 (s, 1H), 7.17 (s, 1H), 7.07- |
| | | | | 6.98 (m, 1H), 6.98-6.94 (m, 1H), 6.94-6.84 |
| | | | | (m, 1H), 5.36 (dd, J = 5.6, 12.4 Hz, 1H), 4.51- |
| | | | | 4.33 (m, 1H), 3.98 (s, 3H), 3.64 (s, 3H), 3.53 (s, |
| | | | | 1H), 3.13-2.83 (m, 6H), 2.77-2.60 (m, 5H), 2.25- |
| | | | | 2.07 (m, 3H), 2.05-1.72 (m, 6H), 1.28-1.10 (m, 2H) |
| I-298 | BAI | CEP | 775.4 | 11.09 (s, 1H), 10.47 (s, 1H), 9.39 (d, J = 52 Hz, |
| | | | | 1H), 8.69 (s, 1H), 8.43 (d, J = 5.2 Hz, 1H), 8.36 |
| | | | | (s, 1H), 7.18 (s, 1H), 7.12-6.97 (m, 1H), 6.96 (s, |
| | | | | 1H), 6.94-6.81 (m, 1H), 5.44-5.23 (m, 1H), 4.56- |
| | | | | 4.29 (m, 1H), 3.99 (s, 3H), 3.64 (s, 3H), 3.50- |
| | | | | 3.38 (m, 4H), 3.12-2.81 (m, 6H), 2.30-2.11 (m, |
| | | | | 4H), 2.05-1.83 (m, 6H), 1.30-1.11 (m, 3H) |
| I-299 | BXL | CEO | 757.3 | 9.70 (s, 1H), 9.41 (s, 1H), 8.79 (s, 1H), 8.68 (s, |
| | | | | 1H), 8.61 (s, 1H), 8.59-8.59 (m, 1H), 7.83 (s, |
| | | | | 1H), 7.71 (s, 1H), 7.54 (s, 1H), 6.96-6.86 (m, |
| | | | | 1H), 6.81 (s, 1H), 6.46 (d, J = 7.2 Hz, 1H), 6.37- |
| | | | | 6.33 (m, 1H), 3.66-3.52 (m, 1H), 3.17-3.13 (m, |
| | | | | 1H), 3.19-3.13 (m, 1H), 3.15 (s, 1H), 3.13-3.04 |
| | | | | (m, 1H), 2.93-2.83 (m, 1H), 2.41-2.26 (m, 4H), |
| | | | | 2.20-2.09 (m, 1H), 1.97-1.86 (m, 4H), 1.51- |
| | | | | 1.48 (m, 1H), 1.43-1.43 (m, 1H), 1.35 (d, J = 10.0 |
| | | | | Hz, 2H), 1.25-1.04 (m, 4H), 0.97-0.81 (m, 1H), |
| | | | | 0.35 (d, J = 12.4 Hz, 2H) |
| I-300 | BXL | CEP | 757.3 | 10.57-10.45 (m, 2H), 9.43 (s, 2H), 8.69 (s, 1H), |
| | | | | 8.53 (s, 1H), 8.43 (d, J = 4.0 Hz, 1H), 8.37 (s, 1H), |
| | | | | 7.77-7.69 (m, 1H), 7.62 (d, J = 9.6 Hz, 1H), 7.29 |
| | | | | (d, J = 7.6 Hz, 1H), 7.2 (s, 1H), 4.46-4.36 (m, |
| | | | | 1H), 3.99 (s, 3H), 3.95-3.87 (m, 1H), 3.73-3.66 |
| | | | | (m, 1H), 3.20-3.10 (m, 5H), 2.98-2.93 (m, 1H), |
| | | | | 2.81-2.72 (m, 4H), 2.20-2.14 (m, 2H), 2.03- |
| | | | | 1.90 (m, 4H), 1.77-1.65 (m, 1H), 1.26-1.13 (m, 4H) |
| I-301 | CAL | BSC | 772.4 | 11.1 (s, 1H), 10.14 (s, 1H), 8.47-8.35 (m, 3H), |
| | | | | 8.25-8.15 (m, 2H), 7.51 (s, 1H), 7.10-6.86 (m, |
| | | | | 3H), 5.44-5.31 (m, 1H), 4.47 (s, 1H), 3.75 (d, J = |
| | | | | 10.0 Hz, 1H), 3.65 (s, 3H), 3.61-3.44 (m, 1H), |
| | | | | 3.27 (s, 1H), 3.22-2.82 (m, 5H), 2.77-2.58 (m, |
| | | | | 3H), 2.41 (s, 3H), 2.27-2.09 (m, 3H), 2.07-1.88 |
| | | | | (m, 5H), 1.64-0.95 (m, 6H) |
| I-302 | BWR | BSC | 772.3 | 11.09 (s, 1H), 10.13 (s, 1H), 8.48-8.33 (m, 3H), |
| | | | | 8.25-8.16 (m, 2H), 7.51 (s, 1H), 7.12-6.82 (m, |
| | | | | 3H), 5.47-5.25 (m, 1H), 4.57-4.36 (m, 1H), 3.65 |
| | | | | (s, 4H), 3.12-2.80 (m, 5H), 2.78-2.62 (m, 3H), |
| | | | | 2.41 (s, 3H), 2.36-2.13 (m, 4H), 2.12-1.91 (m, |
| | | | | 5H), 1.75-1.51 (m, 1H), 1.45-0.98 (m, 6H) |
| I-303 | BWT | BSC | 770.3 | 11.09 (s, 1H), 10.13 (s, 1H), 8.48-8.33 (m, 3H), |
| | | | | 8.26-8.15 (m, 2H), 7.51 (s, 1H), 7.10-6.95 (m, |
| | | | | 2H), 6.89 (d, J = 2.4 Hz, 1H), 5.36 (dd, J = 5.2, |
| | | | | 12.4 Hz, 1H), 4.52-4.41 (m, 1H), 4.15-3.89 (m, |
| | | | | 1H), 3.85-3.68 (m, 1H), 3.63 (s, 3H), 3.21-2.99 |
| | | | | (m, 2H), 2.99-2.79 (m, 2H), 2.76-2.59 (m, 3H), |
| | | | | 2.52 (d, J = 1.6 Hz, 3H), 2.41 (s, 3H), 2.27-2.12 |
| | | | | (m, 3H), 2.11-1.89 (m, 6H), 1.37-1.16 (m, 2H) |
| I-304 | CEQ | BSC | 784.4 | 11.13-10.98 (m, 1H), 10.13 (s, 1H), 8.46-8.35 |
| | | | | (m, 3H), 8.24-8.16 (m, 2H), 7.51 (s, 1H), 7.02- |
| | | | | 6.95 (m, 1H), 6.92-6.86 (m, 1H), 6.82 (d, J = 8.0 |
| | | | | Hz, 1H), 5.28 (dd, J = 5.2, 12.4 Hz, 1H), 4.48- |
| | | | | 4.38 (m, 1H), 3.06-2.91 (m, 4H), 2.88-2.81 (m, |
| | | | | 1H), 2.65-2.54 (m, 7H), 2.40 (s, 3H), 2.24 (d, J = |
| | | | | 6.4 Hz, 2H), 2.16 (d, J = 10.4 Hz, 2H), 2.02- |
| | | | | 1.89 (m, 5H), 1.73-1.61 (m, 1H), 1.20-1.09 (m, |
| | | | | 2H), 1.07-0.99 (m, 2H), 0.97-0.89 (m, 2H) |
| I-307 | BAI | BIZ | 790.3 | 11.11 (s, 1H), 10.77 (s, 1H), 8.57 (s, 1H), 8.50- |
| | | | | 8.39 (m, 3H), 8.24 (d, J = 7.6 Hz, 1H), 7.81 (s, 1H), |
| | | | | 7.03-6.87 (m, 3H), 5.36 (dd, J = 5.2, 12.8 Hz, 1H), |
| | | | | 4.54-4.41 (m, 1H), 3.64 (s, 3H), 3.10-2.79 (m, 8H), 2.62 |
| | | | | (d, J = 19.2 Hz, 3H), 2.26 (d, J = 6.0 Hz, 3H), |
| | | | | 2.17 (d, J = 11.2 Hz, 3H), 2.04-1.90 (m, 5H), 1.68 |
| | | | | (d, J = 7.2 Hz, 1H), 1.16 (d, J = 10.8 Hz, 3H) |
| I-308^{h} | BAI | CEU | 806.3 | 11.8 (s, 1H), 8.89 (s, 1H), 8.48 (d, J = 7.6 Hz, 1H), |
| | | | | 8.38 (s, 1H), 8.13 (t, J = 8.0 Hz, 1H), 8.02 (d, J = |
| | | | | 9.2 Hz, 2H), 7.88 (d, J = 7.6 Hz, 1H), 7.05-6.94 |
| | | | | (m, 2H), 6.60 (d, J = 7.6 Hz, 1H), 5.22 (dd, J = |
| | | | | 5.2, 12.4 Hz, 1H), 4.45-4.42(m, 1H), 3.77 (s, |
| | | | | 3H), 3.05 (d, J = 4.4 Hz, 4H), 2.96 (s, 3H), 2.92 |
| | | | | (d, J = 4.4 Hz, 1H), 2.88-2.82 (m, 1H), 2.79- |
| | | | | 2.71 (m, 1H), 2.44-2.31 (m, 6H), 2.24-2.22 (m, |
| | | | | 1H), 2.13 (d, J = 12.8 Hz, 3H), 2.05-1.93 (m, |
| | | | | 4H), 1.81-1.71 (m, 1H), 1.28-1.16 (m, 2H) |
| I-311 | CFB | CEP | 774.2 | 10.52 (d, J = 11.6 Hz, 2H), 9.50 (d, J = 2.4 Hz, |
| | | | | 1H), 8.69 (s, 1H), 8.57 (s, 1H), 8.50-8.44 (m, |
| | | | | 1H), 8.43-8.37 (m, 1H), 8.34 (s, 1H), 8.25-8.18 |
| | | | | (m, 1H), 7.56 (dd, J = 8.4, 12.4 Hz, 1H), 7.31 (dd, |
| | | | | J = 4.0, 8.4 Hz, 1H), 7.16 (s, 1H), 4.40 (br t, J = |
| | | | | 11.6 Hz, 1H), 3.98 (s, 3H), 3.88-3.80 (m, 1H), |
| | | | | 3.78-3.69 (m, 1H), 3.29 (s, 2H), 3.18-3.04 (m, 4H), |
| | | | | 2.91-2.87 (m, 1H), 2.75-2.70 (m, 2H), 2.30 (s, 1H), |
| | | | | 2.24-2.08 (m, 3H), 2.06-1.96 (m, 2H), 1.96-1.86 |
| | | | | (m, 2H), 1.78-1.66 (m, 1H), 1.25-1.09 (m, 3H) |
| I-312 | CFC | ATJ | 705.2 | 10.50 (s, 1H), 10.26 (s, 1H), 8.69 (s, 1H), 8.51- |
| | | | | 8.38 (m, 2H), 8.36-8.30 (m, 1H), 8.22 (d, J = 7.6 |
| | | | | Hz, 1H), 7.20-7.09 (m, 3H), 6.94 (d, J = 8.4 Hz, |
| | | | | 2H), 4.44-4.31 (m, 1H), 3.98 (s, 3H), 3.70 (t, J = |
| | | | | 6.4 Hz, 2H), 3.23-3.05 (m, 4H), 2.66 (s, 2H), 2.60 (s, |
| | | | | 4H), 2.30-2.19 (m, 2H), 2.15 (d, J = 10.4 Hz, 2H), |
| | | | | 2.04-1.85 (m, 4H), 1.76-1.60 (m, 1H), 1.21-1.06 (m, 2H) |
| I-313 | CFD | ATJ | 706.2 | 10.50 (s, 1H), 10.35 (s, 1H), 8.69 (s, 1H), 8.49- |
| | | | | 8.44 (m, 1H), 8.43-8.38 (m, 1H), 8.34 (s, 1H), |
| | | | | 8.24-8.19 (m, 1H), 8.08 (d, J = 2.8 Hz, 1H), 7.52 |
| | | | | (dd, J = 2.8, 9.2 Hz, 1H), 7.16 (s, 1H), 6.86 (d, J = |
| | | | | 92 Hz, 1H), 4.46-4.30 (m, 1H), 3.98 (s, 3H), |
| | | | | 3.72 (t, J = 6.8 Hz, 2H), 3.50 (s, 5H), 3.30-3.14 |
| | | | | (m, 3H), 2.72 (t, J = 6.8 Hz, 2H), 2.24 (d, J = 6.0 |
| | | | | Hz, 2H), 2.16 (d, J = 11.6 Hz, 2H), 2.05-1.82 (m, |
| | | | | 4H), 1.69 (s, 1H), 1.22-1.05 (m, 2H) |
| I-314 | CFE | BCN | 778.2 | 0.97-1.12 (3H, m) 1.48-1.60 (3H, m) 1.92-2.04 |
| | | | | (3H, m) 2.14 (2H, d, J = 11.6 Hz) 2.20 (1H, s) 2.22 |
| | | | | (3H, s) 2.55-2.60 (4H, m) 2.80-2.92 (1H, m) |
| | | | | 2.98-3.07 (1H, m) 3.38-3.48 (2H, m) 4.03 (3H, |
| | | | | s) 5.05 (1H, dd, J = 12.8, 5.2 Hz) 6.75 (1H, t, J = 4.4 |
| | | | | Hz) 7.04 (1H, d, J = 7.2 Hz) 7.08-7.13 (1H, m) |
| | | | | 7.57-7.63 (1H, m) 7.70 (1H, s) 8.24 (1H, d, J = 7.6 |
| | | | | Hz) 8.39-8.44 (1H, m) 8.46-8.50 (1H, m) 9.00 |
| | | | | (1H, s) 10.50 (1H, s) 11.07 (1H, s) |
| I-315 | CBP | BCN | 792.4 | 11.08 (s, 1H), 10.50 (s, 1H), 9.00 (s, 1H), 8.52- |
| | | | | 8.45 (m, 1H), 8.45-8.39 (m, 1H), 8.27-8.20 (m, |
| | | | | 1H), 7.69 (s, 1H), 7.63-7.55 (m, 1H), 7.11 (d, J = |
| | | | | 8.4 Hz, 1H), 7.02 (d, J = 7.2 Hz, 1H), 6.72 (t, J = |
| | | | | 5.2 Hz, 1H), 5.08-5.00 (m, 1H), 4.03 (s, 3H), 3.07- |
| | | | | 2.97 (m, 1H), 2.93-2.80 (m, 2H), 2.57-2.55 |
| | | | | (m, 2H), 2.41 (d, J = 5.2 Hz, 2H), 2.23-2.07 (m, |
| | | | | 7H), 2.03-1.95 (m, 1H), 1.94-1.85 (m, 2H), 1.74 |
| | | | | (quind, J = 6.0, 12.8 Hz, 2H), 1.62-1.45 (m, 3H), |
| | | | | 1.28-1.11 (m, 1H), 1.10-0.93 (m, 2H) |
| I-317 | BRZ | BRR | 755.5 | 10.50 (s, 1H), 10.19 (s, 1H), 9.06(d, J = 7.6 Hz, |
| | | | | 2H), 8.60 (s, 1H), 8.48 (d, J = 7.6 Hz, 2H), 8.41 |
| | | | | (t, J = 8.0 Hz, 1H), 8.26 (s, 1H), 8.23 (d, J = 8.4 |
| | | | | Hz, 1H), 8.14 (s, 1H), 7.79(d, J = 9.2 Hz, 1H), 7.69- |
| | | | | 7.65 (m, 1H), 7.43 (d, J = 2.4 Hz, 1H), 4.60- |
| | | | | 4.52 (m, 1H), 4.01 (d, J = 12.0 Hz, 2H), 3.93-3.86 |
| | | | | (m, 1H), 3.73-3.67 (m, 1H), 2.99-2.81 (m, 4H), |
| | | | | 2.80-2.70 (m, 2H), 2.38-2.30 (m, 3H), 2.19 (br |
| | | | | d, J = 9.8 Hz, 2H), 1.97 (t, J = 10.4 Hz, 5H), 1.92- |
| | | | | 1.85 (m, 2H), 1.64 (s, 3H), 1.19-1.10 (m, 2H) |
| I-318 | BRY | BRR | 726.3 | 10.53 (s, 1H), 10.20 (s, 1H), 9.26 (s, 1H), 9.07 (s, |
| | | | | 1H), 8.62 (s, 1H), 8.50-8.47 (m, 3H), 8.44-8.39 |
| | | | | (m, 1H), 8.23 (d, J = 7.8 Hz, 1H), 8.07 (s, 1H), |
| | | | | 7.92 (d, J = 8.4 Hz, 1H), 7.79 (dd, J = 1.2, 8.8 Hz, |
| | | | | 1H), 4.64-4.53 (m, 1H), 3.96-3.89 (m, 1H), 3.77- |
| | | | | 3.68 (m, 1H), 3.03 (d, J = 11.2 Hz, 2H), 2.99- |
| | | | | 2.91 (m, 1H), 2.76 (td, J = 5.6, 16.4 Hz, 2H), 2.26- |
| | | | | 2.18 (m, 4H), 2.12-1.95 (m, 6H), 1.91-1.78 |
| | | | | (m, 4H), 1.75-1.65 (m, 1H), 1.25-1.08 (m, 1H) |
| I-319 | BRZ | BTW | 754.2 | 10.50 (s, 1H), 10.35 (s, 1H), 9.07 (s, 1H), 8.41- |
| | | | | 8.34 (m, 3H), 8.29 (d, J = 0.8 Hz, 1H), 8.26 (s, |
| | | | | 1H), 8.19-8.15 (m, 1H), 7.78 (d, J = 9.6 Hz, 1H), |
| | | | | 7.68-7.65 (m, 1H), 7.62-7.58 (m, 1H), 7.56- |
| | | | | 7.53 (m, 1H), 7.42 (d, J = 2.4 Hz, 1H), 4.47-4.40 |
| | | | | (m, 1H), 3.98 (d, J = 12.4 Hz, 2H), 3.93-3.86 |
| | | | | (m, 1H), 3.73-3.67 (m, 1H), 2.97-2.93 (m, 1H), |
| | | | | 2.85 (s, 2H), 2.76 (d, J = 5.6 Hz, 1H), 2.28 (d, J = |
| | | | | 6.4 Hz, 2H), 2.23 (s, 3H), 2.18-2.14 (m, 2H), 2.00- |
| | | | | 1.93 (m, 4H), 1.87-1.76 (m, 3H), 1.62-1.54 |
| | | | | (m, 3H), 1.15-1.08 (m, 2H) |
| I-320 | BRZ | ATJ | 784.2 | 10.50 (s, 2H), 9.07 (s, 1H), 8.68 (s, 1H), 8.48- |
| | | | | 8.37 (m, 2H), 8.33 (s, 1H), 8.26 (s, 1H), 8.23- |
| | | | | 8.19 (m, 1H), 7.78 (d, J = 9.6 Hz, 1H), 7.70-7.63 |
| | | | | (m, 1H), 7.42 (d, J = 2.0 Hz, 1H), 7.15 (s, 1H), |
| | | | | 4.40-4.32 (m, 1H), 4.05-3.98 (m, 2H), 3.98 (s, |
| | | | | 3H), 3.93-3.86 (m, 1H), 3.73-3.67 (m, 1H), 2.98- |
| | | | | 2.90 (m, 1H), 2.84 (t, J = 12.0 Hz, 2H), 2.78- |
| | | | | 2.71 (m, 1H), 2.28 (d, J = 6.8 Hz, 2H), 2.23 (s, |
| | | | | 3H), 2.16-2.12 (m, 2H), 2.01-1.89 (m, 4H), 1.88- |
| | | | | 1.80 (m, 3H), 1.62-1.53 (m, 3H), 1.15-1.07 (m, 2H) |
| I-321 | BRY | BTW | 725.1 | 10.54 (s, 1H), 10.34 (s, 1H), 9.26 (s, 1H), 8.48 (s, |
| | | | | 1H), 8.40 (d, J = 92 Hz, 1H), 8.36 (s, 1H), 8.30 (s, |
| | | | | 1H), 8.18 (s, 1H), 8.07 (s, 1H), 7.90 (s, 1H), 7.61 |
| | | | | (s, 1H), 7.56 (s, 1H), 7.55 (s, 1H), 4.49-4.47 (m, |
| | | | | 1H), 3.94-3.93 (m, 1H), 3.74-3.73 (m, 1H), 3.05- |
| | | | | 2.96 (m, 3H), 2.78-2.52 (m, 2H), 2.26-2.4 (m, |
| | | | | 4H), 2.09-1.99 (m, 2H), 1.98-1.97 (m, 8H), 1.87- |
| | | | | 1.70 (m, 1H), 1.20-1.14 (m, 2H) |
| I-322 | BRY | ATJ | 755.2 | 10.53 (d, J = 12.4 Hz, 2H), 9.27 (s, 1H), 8.69 (s, |
| | | | | 1H), 8.49 (s, 1H), 8.48-8.43 (m, 1H), 8.41-8.36 |
| | | | | (m, 1H), 8.34 (s, 1H), 8.22 (d, J = 7.6 Hz, 1H), |
| | | | | 8.06 (s, 1H), 7.95 (d, J = 8.8 Hz, 1H), 7.79-7.70 |
| | | | | (m, 1H), 4.44-4.38 (m, 1H), 3.98 (s, 3H), 3.97- |
| | | | | 3.90 (m, 1H), 3.74-3.71 (m, 1H), 3.40-3.34 (m, |
| | | | | 4H), 2.96-2.78 (m, 3H), 2.49-2.40 (m, 2H), 2.20- |
| | | | | 2.16 (m, 2H), 2.00-1.94 (m, 8H), 1.92-1.84 |
| | | | | (m, 1H), 1.23-1.14 (m, 2H) |
| I-323 | BQM | BVH | 714.3 | 11.09 (s, 1H), 10.60 (s, 1H), 8.43-8.38 (m, 2H), |
| | | | | 8.35-8.28 (m, 3H), 7.61 (s, 2H), 6.93-6.81 (m, |
| | | | | 2H), 5.39-5.30 (m, 1H), 4.55-4.42 (m, 1H), 3.65 |
| | | | | (s, 3H), 3.58 (s, 1H), 3.29 (s, 2H), 3.21-2.98 (m, |
| | | | | 2H), 2.94-2.84 (m, 1H), 2.78-2.68 (m, 1H), 2.65- |
| | | | | 2.58 (m, 1H), 2.48-2.47 (m, 4H), 2.25-2.15 |
| | | | | (m, 3H), 1.98 (d, J = 6.0 Hz, 9H), 1.34-0.93 (m, 3H) |
| I-325 | AZK | BWH | 784.3 | 11.09 (s, 1H), 10.52 (s, 1H), 9.32 (s, 1H), 9.24 (s, |
| | | | | 1H), 8.77 (s, 1H), 8.56 (s, 1H), 7.98 (s, 1H), 7.72 |
| | | | | (s, 1H), 7.08-6.92 (m, 3H), 5.37 (dd, J = 5.4, 12.4 |
| | | | | Hz, 1H), 4.59-4.46 (m, 1H), 3.59 (s, 3H), 3.05- |
| | | | | 2.98 (m, 2H), 2.94-2.84 (m, 1H), 2.77-2.62 (m, 2H), |
| | | | | 2.24 (d, J = 6.4 Hz, 2H), 2.18 (d, J = 10.4 Hz, |
| | | | | 2H), 2.14-2.05 (m, 2H), 2.04-1.95 (m, 4H), 1.95-1.90 |
| | | | | (m, 1H), 1.88-1.59 (m, 6H), 1.15 (q, J = 12.2 Hz, 2H) |
| I-326 | CBD | AGL | 840.4 | 12.36 (s, 1H), 11.12 (s, 1H), 8.71 (s, 1H), 8.50- |
| | | | | 8.41 (m, 1H), 8.40-8.28 (m, 2H), 8.17-8.13 (m, |
| | | | | 1H), 7.57 (s, 1H), 7.23-7.06 (m, 2H), 7.05-6.97 |
| | | | | (m, 1H), 5.94 (s, 1H), 5.39 (dd, J = 5.2, 12.8 Hz, |
| | | | | 1H), 4.50-4.35 (m, 1H), 3.65 (s, 3H), 3.62 (s, |
| | | | | 1H), 2.93-2.84 (m, 1H), 2.36-2.29 (m, 1H), 2.14 |
| | | | | (d, J = 9.6 Hz, 2H), 2.05-1.99 (m, 1H), 1.97- |
| | | | | 1.86 (m, 4H), 1.61 (s, 6H), 1.20-1.07 (m, 2H) |
| I-327 | CBD | ATJ | 812.3 | 11.12 (s, 1H), 10.50 (s, 1H), 8.73 (s, 1H), 8.49 (s, |
| | | | | 2H), 8.32 (s, 1H), 8.22 (d, J = 7.6 Hz, 1H), 7.19- |
| | | | | 7.09 (m, 3H), 7.06-6.99 (m, 1H), 5.39 (dd, J = |
| | | | | 5.6, 12.4 Hz, 1H), 4.43-4.31 (m, 1H), 3.98 (s, 3H), |
| | | | | 3.75-3.47 (m, 6H), 2.96-2.81 (m, 2H), 2.79-2.55 (m, |
| | | | | 7H), 2.26-2.07 (m, 4H), 2.05-1.99 (m, 1H), 1.99-1.82 |
| | | | | (m, 5H), 1.73-1.57 (m, 1H), 1.22-1.05 (m, 2H) |
| I-328 | CAR | BTW | 758.3 | 11.09 (s, 1H), 10.36 (s, 1H), 8.43-8.33 (m, 3H), |
| | | | | 8.30 (s, 1H), 8.17 (dd, J = 1.2, 7.2 Hz, 1H), 7.64- |
| | | | | 7.59 (m, 1H), 7.57-7.52 (m, 1H), 7.03-6.96 (m, |
| | | | | 2H), 6.89 (d, J = 5.6 Hz, 1H), 5.35 (dd, J = 5.2, |
| | | | | 12.8 Hz, 1H), 4.51-4.41 (m, 1H), 3.66 (s, 3H), |
| | | | | 3.29 (s, 2H), 3.26-3.12 (m, 4H), 3.04-2.82 (m, |
| | | | | 4H), 2.76-2.68 (m, 1H), 2.65-2.56 (m, 2H), 2.19 |
| | | | | (d, J = 10.4 Hz, 2H), 2.04-1.90 (m, 7H), 1.79- |
| | | | | 1.62 (m, 1H), 1.26-1.12 (m, 2H) |
| I-329 | BSN | BWF | 763.5 | 10.55 (s, 1H), 9.64 (s, 1H), 8.86 (s, 1H), 8.82 (d, |
| | | | | J = 2.0 Hz, 1H), 8.68 (d, J = 2.0 Hz, 1H), 8.59 (s, |
| | | | | 1H), 8.56 (s, 1H), 8.53 (d, J = 4.0 Hz, 1H), 8.34 |
| | | | | (d, J = 7.2 Hz, 1H), 8.10 (s, 1H), 7.91-7.85 (d, |
| | | | | 1H), 7.81 (t, J = 7.6 Hz, 1H), 7.66 (d, J = 7.2 Hz, |
| | | | | 1H), 6.89 (d, J = 4.0 Hz, 1H), 4.65-4.51 (m, 1H), |
| | | | | 4.01-3.83 (m, 2H), 3.72 (m, 2H), 3.14-2.93 (m, |
| | | | | 3H), 2.81-2.74 (m, 1H), 2.53 (d, J = 2.0 Hz, 2H), |
| | | | | 2.32-2.15 (m, 3H), 2.13-1.85 (m, 9H), 1.83-1.75 (m, |
| | | | | 1H), 1.37 (d, J = 6.4 Hz, 6H), 1.30-1.20 (m, 2H) |
| I-330 | CBE | ATJ | 770.3 | 10.51 (s, 1H), 9.54-9.52 (m, 1H), 8.70 (s, 1H), |
| | | | | 8.61-8.59 (m, 1H), 8.49-8.44 (m, 1H), 8.43- |
| | | | | 8.38 (m, 1H), 8.35 (s, 1H), 8.22 (d, J = 7.6 Hz, |
| | | | | 1H), 7.76-7.73 (m, 2H), 7.38-7.37 (m, 1H), 7.16 |
| | | | | (s, 1H), 4.50-4.37 (m, 1H), 3.99 (s, 3H), 3.95- |
| | | | | 3.84 (m, 1H), 3.81-3.65 (m, 3H), 3.63-3.46 (m, |
| | | | | 3H), 3.24-3.21 (m, 3H), 3.12-3.03 (m, 4H), 2.92- |
| | | | | 2.87 (m, 1H), 2.78-2.64 (m, 1H), 2.52-2.50 (m, 1H), |
| | | | | 2.19 (m, 2H), 2.08-1.87 (m, 5H), 1.42-1.10 (m, 2H) |
| I-331 | CBG | ATJ | 770.3 | 10.53 (s, 1H), 10.51 (s, 1H), 9.78-9.06 (m, 1H), |
| | | | | 8.71 (s, 1H), 8.52 (s, 1H), 8.48-8.44 (m, 1H), |
| | | | | 8.43-8.38 (m, 1H), 8.36 (s, 1H), 8.22 (d, J = 7.6 |
| | | | | Hz, 1H), 7.87-7.69 (m, 1H), 7.68-7.58 (m, 1H), |
| | | | | 7.16 (s, 1H), 4.53-4.38 (m, 1H), 3.99 (s, 3H), |
| | | | | 3.95-3.86 (m, 1H), 3.85-3.74 (m, 1H), 3.73- |
| | | | | 3.41 (m, 4H) 3.27-3.08 (m, 3H), 3.02-2.92 (m, |
| | | | | 1H), 2.90-2.69 (m, 2H), 2.55 (s, 3H), 2.54-2.50 |
| | | | | (m, 2H), 2.26-2.14 (m, 2H), 2.13-1.83 (m, 5H), |
| | | | | 1.43-1.15 (m, 2H) |
| I-332 | CBI | ATJ | 786.4 | 10.52 (br d, J = 9.2 Hz, 2H), 9.67 (br s, 1H), 8.70 |
| | | | | (s, 1H), 8.49-8.38 (m, 3H), 8.35 (s, 1H), 8.22 (d, |
| | | | | J = 7.6 Hz, 1H), 7.91-7.82 (m, 1H), 7.74 (br d, J = |
| | | | | 9.2 Hz, 1H), 7.16 (s, 1H), 4.47-4.36 (m, 1H), |
| | | | | 3.99 (s, 6H), 3.95-3.85 (m, 2H), 3.72-3.67 (m, |
| | | | | 2H), 3.00-2.91 (m, 3H), 2.81-2.71 (m, 2H), 2.53- |
| | | | | 2.52 (m, 4H), 2.24-2.14 (m, 3H), 2.07-1.88 |
| | | | | (m, 5H), 1.29-1.13 (m, 2H) |
| I-333 | CBL | CBK | 751.5 | 11.1 (s, 1H), 8.19-8.12 (m, 1H), 7.79 (t, J = 8.0 |
| | | | | Hz, 1H), 7.52 (d, J = 8.0 Hz, 1H), 7.35 (s, 1H), |
| | | | | 7.28 (d, J = 7.6 Hz, 2H), 7.14-7.06 (m, 2H), 6.87 |
| | | | | (s, 1H), 5.44-5.34 (m, 1H), 4.67-4.47 (m, 1H), |
| | | | | 4.36-4.29 (m, 1H), 4.25-4.13 (m, 1H), 3.72 (s, |
| | | | | 3H), 3.54 (s, 3H), 3.27 (s, 3H), 2.89 (s, 2H), 2.78- |
| | | | | 2.57 (m, 4H), 2.22-1.94 (m, 4H), 1.92-1.77 |
| | | | | (m, 4H), 1.70-1.52 (m, 1H), 1.30-0.98 (m, 9H) |
| I-334 | CBM | CBN | 859.2 | 12.36 (s, 1H), 11.16-11.02 (m, 1H), 8.71 (s, 1H), |
| | | | | 8.49-8.42 (m, 1H), 8.39-8.34 (m, 2H), 8.16 (d, |
| | | | | J = 7.8 Hz, 1H), 7.60-7.55 (m, 2H), 7.10 (d, J = |
| | | | | 8.6 Hz, 1H), 7.01 (d, J = 7.0 Hz, 1H), 5.94 (s, 1H), |
| | | | | 5.05 (dd, J = 5.4, 13.2 Hz, 1H), 4.46-4.35 (m, |
| | | | | 1H), 2.97-2.79 (m, 3H), 2.28-2.24 (m, 2H), 2.13 |
| | | | | (s, 3H), 2.04-1.98 (m, 2H), 1.96-1.89 (m, 4H), |
| | | | | 1.62 (s, 6H), 1.43-1.39 (m, 2H), 1.38-1.30 (m, |
| | | | | 6H), 1.12-1.03 (m, 1H), 1.11-1.03 (m, 1H) |
| I-335 | CBO | AGL | 841.3 | 12.36 (s, 1H), 11.02 (s, 1H), 8.72 (s, 1H), 8.48- |
| | | | | 8.41 (m, 1H), 8.40-8.32 (m, 2H), 8.19-8.13 (m, |
| | | | | 1H), 7.61-7.55 (m, 1H), 7.27 (t, J = 7.8 Hz, 1H), |
| | | | | 6.95 (d, J = 7.4 Hz, 1H), 6.64 (d, J = 7.9 Hz, 1H), |
| | | | | 5.94 (s, 1H), 5.78 (br d, J = 6.6 Hz, 1H), 5.16- |
| | | | | 5.09 (m, 1H), 4.47-4.37 (m, 1H), 4.30-4.11 (m, |
| | | | | 2H), 3.99-3.88 (m, 1H), 2.99-2.88 (m, 1H), 2.63 |
| | | | | (br d, J = 18.5 Hz, 3H), 2.31-2.20 (m, 5H), 2.13 |
| | | | | (br d, J = 6.4 Hz, 4H), 2.08-2.00 (m, 1H), 1.98- |
| | | | | 1.86 (m, 4H), 1.70-1.59 (m, 12H), 1.56 (brs, 2H), |
| | | | | 1.18-1.06 (m, 2H) |
| I-336 | CBP | CBN | 803.2 | 12.37 (s, 1H), 11.10 (s, 1H), 8.72 (s, 1H), 8.50- |
| | | | | 8.42 (m, 1H), 8.42-8.32 (m, 2H), 8.21-8.11 (m, |
| | | | | 1H), 7.67-7.52 (m, 2H), 7.20-7.12 (m, 1H), 7.10- |
| | | | | 7.02 (m, 1H), 6.76 (d, J = 6.4 Hz, 1H), 5.97- |
| | | | | 5.89 (m, 1H), 5.06 (dd, J = 5.5, 12.6 Hz, 1H), 4.46- |
| | | | | 4.39 (m, 1H), 3.44-3.41 (m, 2H), 2.92-2.88 |
| | | | | (m, 2H), 2.84 (d, J = 5.2 Hz, 5H), 2.53-2.52 (m, |
| | | | | 3H), 2.15-2.10 (m, 3H), 1.96-1.88 (m, 5H), 1.63 |
| | | | | (s, 6H), 1.27-1.09 (m, 3H) |
| I-337 | CBQ | CBN | 803.3 | 12.36 (s, 1H), 8.71 (s, 1H), 8.48-8.42 (m, 1H), |
| | | | | 8.41-8.32 (m, 3H), 8.16 (d, J = 7.6 Hz, 1H), 7.63- |
| | | | | 7.53 (m, 2H), 7.12 (d, J = 8.4 Hz, 1H), 7.03 (d, J = |
| | | | | I2 Hz, 1H), 6.59 (br t, J = 5.6 Hz, 1H), 6.03- |
| | | | | 5.85 (m, 1H), 5.13-4.98 (m, 1H), 4.49-4.34 (m, |
| | | | | 1H), 2.88 (ddd, J = 4.4, 14.0, 17.2 Hz, 2H), 2.65- |
| | | | | 2.59 (m, 3H), 2.58-2.54 (m, 4H), 2.13 (d, J = 11.6 |
| | | | | Hz, 2H), 2.08-1.98 (m, 2H), 1.97-1.83 (m, 4H), |
| | | | | 1.64 (s, 2H), 1.62 (s, 6H), 1.59-1.51 (m, 3H), |
| | | | | 1.16 (d, J = 10.0 Hz, 2H) |
| I-339 | CBO | ATJ | 813.1 | 1.06-1.17 (m, 2 H) 1.51-1.73 (m, 8 H) 1.78- |
| | | | | I.99 (m, 5 H) 1.99-2.07 (m, 1 H) 2.09-2.19 (m, |
| | | | | 4 H) 2.21-2.32 (m, 5 H) 2.59-2.65 (m, 1 H) 2.85- |
| | | | | 3.03 (m, 2 H) 3.93 (d, J = 12 Hz, 1 H) 3.98 (s, 3 |
| | | | | H) 4.09-4.29 (m, 2 H) 4.31-4.42 (m, 1 H) 5.12 |
| | | | | (dd, J = 13.38, 5.2 Hz, 1 H) 6.63 (d, J = 7.6 Hz, 1 H) |
| | | | | 6.94 (d, J = 7.2 Hz, 1 H) 7.15 (s, 1 H) 7.27 (t, J = 7.6 |
| | | | | Hz, 1 H) 8.15 (d, J = 3.6 Hz, 1 H) 8.22 (dd, J = 7.63, |
| | | | | 0.88 Hz, 1 H) 8.32 (s, 1 H) 8.37-8.43 (m, 1 H) |
| | | | | 8.44-8.49 (m, 1 H) 8.68 (s, 1 H) 10.50 (s, 1 H) |
| | | | | 11.01 (s, 1 H) |
| I-340 | CBT | AGL | 901.3 | 12.35 (s, 1H), 11.08 (s, 1H), 8.71 (s, 1H), 8.51- |
| | | | | 8.41 (m, 1H), 8.39-8.32 (m, 2H), 8.16 (d, J = 7.6 |
| | | | | Hz, 1H), 7.61-7.53 (m, 2H), 7.16 (d, J = 8.8 Hz, |
| | | | | 1H), 7.03 (d, J = 7.2 Hz, 1H), 6.19 (d, J = 8.4 Hz, |
| | | | | 1H), 5.93 (s, 1H), 5.03 (dd, J = 5.2, 12.4 Hz, 1H), |
| | | | | 4.48-4.37 (m, 1H), 3.64-3.51 (m, 1H), 3.46 (t, |
| | | | | J = 6.0 Hz, 2H), 2.97-2.80 (m, 1H), 2.72-2.65 |
| | | | | (m, 1H), 2.58 (d, J = 2.8 Hz, 2H), 2.40-2.29 (m, |
| | | | | 3H), 2.17-2.12 (m, 6H), 2.05-1.88 (m, 9H), 1.71-1.52 |
| | | | | (m, 9H), 1.34 (t, J = 8.8 Hz, 4H), 1.17-1.02 (m, 2H) |
| I-342 | AXB | ATJ | 855.4 | 1.02-1.12 (m, 2 H) 1.24 (d, J = 4.4 Hz, 1 H) 1.29- |
| | | | | 1.37 (m, 3 H) 1.48-1.55 (m, 2 H) 1.59-1.64 |
| | | | | (m, 2 H) 1.66-1.74 (m, 3 H) 1.85-1.96 (m, 6 H) |
| | | | | 2.00-2.07 (m, 2 H) 2.10-2.15 (m, 2 H) 2.18 (s, |
| | | | | 3 H) 2.22 (d, J = 5.6 Hz, 3 H) 2.59-2.61 (m, 2 H) |
| | | | | 2.87 (d, J = 10.4 Hz, 1 H) 3.98 (s, 3 H) 4.06-4.12 |
| | | | | (m, 1 H)4.31-4.40 (m, 1 H) 5.03-5.09 (m, 1 H) |
| | | | | 6.43 (d, J = 6.4 Hz, 1 H) 7.00 (d, J = 8.4 Hz, 1 H) |
| | | | | 7.04-7.08 (m, 1 H) 7.15 (s, 1 H) 7.56-7.63 (m, |
| | | | | 1 H) 8.22 (d, J = 7, 6 Hz.1 H) 8.33 (s, 1 H) 8.40 (t, |
| | | | | J = 7.6 Hz, 1 H) 8.45-8.48 (m, 1 H) 8.68 (s, 1 H) |
| | | | | 10.50 (s, 1 H) 11.10 (s, 1 H) |
| I-343 | AJF | CBW | 863.3 | 12.55-12.33 (m, 1H), 11.20-10.96 (m, 1H), 9.03 |
| | | | | (d, J = 8.4 Hz, 1H), 8.87-8.77 (m, 1H), 8.53- |
| | | | | 8.23 (m, 3H), 8.21-8.10 (m, 1H), 8.03-7.96 (m, |
| | | | | 1H), 7.68 (dd, J = 9.6, 1.2 Hz, 1H), 7.60-7.41 (m, |
| | | | | 3H), 7.17-6.96 (m, 2H), 6.53-6.38 (m, 1H), 6.13- |
| | | | | 5.96 (m, 1H), 5.13-4.93 (m, 1H), 2.31 (d, J = |
| | | | | 9.6 Hz, 6H), 2.10-1.76 (m, 7H), 1.64 (d, J = 8.0 |
| | | | | Hz, 6H), 1.57-1.40 (m, 6H), 1.31-1.15 (m, 2H) |
| I-345 | AJF | CBZ | 843.7 | 12.34 (s, 1H), 11.11-11.03 (m, 1H), 8.75-8.65 |
| | | | | (m, 1H), 8.47-8.42 (m, 1H), 8.38-8.30 (m, 2H), |
| | | | | 8.18-8.11 (m, 1H), 7.59-7.52 (m, 2H), 7.11- |
| | | | | 6.99 (m, 2H), 6.44 (s, 1H), 5.93 (s, 1H), 5.04 (dd, |
| | | | | J = 12.8, 4.0 Hz, 1H), 4.38 (t, J = 6.8 Hz, 2H), |
| | | | | 2.93-2.82 (m, 2H), 2.69-2.64 (m, 1H), 2.22-2.17 |
| | | | | (m, 4H), 2.06-1.98 (m, 2H), 1.91-1.80 (m, 5H), 1.62 |
| | | | | (s, 6H), 1.53-1.40 (m, 10H), 1.25-1.14 (m, 3H) |
| I-347 | CCB | AGL | 887.2 | 12.36 (s, 1H), 11.08 (s, 1H), 8.71 (s, 1H), 8.47- |
| | | | | 8.43 (m, 1H), 8.40-8.32 (m, 2H), 8.17-8.14 (m, |
| | | | | 1H), 7.65-7.59 (m, 1H), 7.56 (s, 1H), 7.29 (d, J = |
| | | | | 6.8 Hz, 1H), 7.13 (d, J = 8.4 Hz, 1H), 5.93 (s, |
| | | | | 1H), 5.12-5.05 (m, 1H), 4.46-4.36 (m, 1H), 4.33- |
| | | | | 4.25 (m, 1H), 4.05-3.94 (m, 1H), 3.36-3.34 |
| | | | | (m, 2H), 2.95 (s, 3H), 2.92-2.83 (m, 1H), 2.62- |
| | | | | 2.53 (m, 2H), 2.43-2.37 (m, 2H), 2.34-2.25 (m, |
| | | | | 5H), 2.22-2.16 (m, 5H), 2.15-2.09 (m, 2H), 2.06- |
| | | | | 1.99 (m, 1H), 1.97-1.88 (m, 4H), 1.71-1.64 |
| | | | | (m, 2H), 1.61 (s, 6H), 1.16-1.03 (m, 2H) |
| I-348 | CCC | AGL | 873.6 | 12.35 (s, 1H), 11.05 (s, 1H), 8.71 (s, 1H), 8.47- |
| | | | | 8.41 (m, 1H), 8.39-8.33 (m, 2H), 8.16 (d, J = 8.4 |
| | | | | Hz, 1H), 7.61-7.53 (m, 2H), 7.44 (d, J = 5.2 Hz, |
| | | | | 1H), 6.87-6.69 (m, 2H), 5.93 (s, 1H), 5.02 (dd, J = |
| | | | | 12.8, 5.2 Hz, 1H), 4.51-4.35 (m, 1H), 4.13 (t, |
| | | | | J = 5.6 Hz, 1H), 4.05-3.97 (m, 1H), 3.38 (s, 2H), |
| | | | | 3.36 (s, 2H), 2.94-2.80 (m, 1H), 2.58 (s, 2H), 2.55- |
| | | | | 2.54 (m, 2H), 2.36 (d, J = 5.2 Hz, 2H), 2.27 (d, |
| | | | | J = 2.0 Hz, 3H), 2.20 (s, 5H), 2.01-1.86 (m, 5H), 1.70 |
| | | | | (d, J = 5.6 Hz, 2H), 1.61 (s, 6H), 1.19-1.07 (m, 2H) |
| I-349 | CCE | AGL | 859.4 | 12.36 (s, 1H), 11.10 (s, 1H), 8.71 (s, 1H), 8.49- |
| | | | | 8.41 (m, 1H), 8.41-8.32 (m, 2H), 8.16 (d, J = 7.6 |
| | | | | Hz, 1H), 7.66-7.53 (m, 2H), 7.08 (d, J = 7.2 Hz, |
| | | | | 1H), 6.92 (d, J = 8.4 Hz, 1H), 6.51 (d, J = 5.6 Hz, |
| | | | | 1H), 5.94 (s, 1H), 5.05 (dd, J = 12.8, 5.6 Hz, 1H), |
| | | | | 4.47-4.38 (m, 1H), 4.20-4.12 (m, 2H), 3.43-3.41 (m, |
| | | | | 2H), 2.91-2.86 (m, 1H), 2.60 (d, J = 3.2 Hz, 2H), |
| | | | | 2.38 (dd, J = 7.6, 4.4 Hz, 2H), 2.33 (s, 1H), 2.26 |
| | | | | (d, J = 6.4 Hz, 3H), 2.22 (s, 3H), 2.17-2.11 (m, |
| | | | | 2H), 2.06-1.84 (m, 1H), 1.62 (s, 8H), 1.16-1.03 (m, 2H) |
| I-350^{f} | AOQ | CBW | 867.3 | 12.45 (s, 1H), 11.09 (s, 1H), 9.03 (s, 1H), 8.81 (s, |
| | | | | 1H), 8.46 (d, J = 7.6 Hz, 1H), 8.38 (t, J = 7.6 Hz, |
| | | | | 1H), 8.17 (d, J = 7.6 Hz, 1H), 8.02 (d, J = 8.4 Hz, |
| | | | | 2H), 7.68 (s, 1H), 7.58-7.47 (m, 3H), 7.05 (d, J = |
| | | | | 7.2 Hz, 1H), 6.86 (d, J = 8.4 Hz, 1H), 6.48 (d, J = |
| | | | | 5.6 Hz, 1H), 6.04 (s, 1H), 5.05 (J = 5.2, 12.8 Hz, |
| | | | | 1H), 4.16-4.04 (m, 2H), 3.53 (s, 3H), 2.95-2.81 |
| | | | | (m, 2H), 2.60 (d, J = 2.0 Hz, 2H), 2.43 (t, J = 7.2 |
| | | | | Hz, 3H), 2.33 (d, J = 4.8 Hz, 2H), 2.21 (d, J = 6.0 |
| | | | | Hz, 2H), 2.16 (s, 2H), 2.09-1.94 (m, 2H), 1.75- |
| | | | | 1.69 (m, 2H), 1.65 (s, 6H) |
| I-351 | CCC | ALU | 865.4 | 11.09 (s, 1H), 10.49 (s, 1H), 8.55 (s, 1H), 8.50- |
| | | | | 8.36 (m, 3H), 8.22(dd, J = 8.0, 0.8 Hz, 1H), 7.97(s, |
| | | | | 1H), 7.58(dd, J = 8.4, 7.2 Hz, 1H), 7.41-7.10(m, |
| | | | | 1H), 7.07(d, J = 7.2 Hz, 1H), 6.92(d, J = 8, 4 Hz, |
| | | | | 1H), 6.50 (d, J = 5.6 Hz, 1H), 5.05 (dd, J = 12.8, 5.2 |
| | | | | Hz, 1H), 4.62-4.44(m, 1H), 4.25-4.06(m, 2H), |
| | | | | 3.37(s, 2H), 2.94-2.80(m, 1H), 2.56(s, 2H), 2.41- |
| | | | | 2.36(m, 3H), 2.34-2.32(m, 1H), 2.29-2.10(m, |
| | | | | 10H), 2.06-1.99(m, 1H), 1.98-1.86(m, 4H), 1.68- |
| | | | | 1.53(m, 3H), 1.20-1.03(m, 2H) |
| I-353 | CCC | BRR | 816.7 | 11.10 (s, 1H), 10.20 (s, 1H), 9.06 (s, 1H), 8.60 (s, |
| | | | | 1H), 8.52-8.46 (m, 2H), 8.42 (t, J = 7.6 Hz, 1H), |
| | | | | 8.24 (d, J = 8.0 Hz, 1H), 7.64-7.54 (m, 1H), 7.08 |
| | | | | (d, J = 7.2 Hz, 1H), 6.92 (d, J = 8.4 Hz, 1H), 6.51 |
| | | | | (d, J = 6.0 Hz, 1H), 5.06 (dd, J = 12.4, 5.6 Hz, |
| | | | | 1H), 4.66-4.45 (m, 1H), 4.26-4.05 (m, 2H), 3.00- |
| | | | | 2.79 (m, 1H), 2.72-2.64 (m, 1H), 2.64-2.53 |
| | | | | (m, 3H), 2.31-2.12 (m, 11H), 2.09-1.88 (m, 6H), |
| | | | | 1.77-1.47 (m, 4H), 1.14 (q, J = 11.6 Hz, 2H) |
| I-354 | CCC | BRP | 815.5 | 11.18-11.02 (m, 1H), 10.36 (s, 1H), 8.40 (s, 3H), |
| | | | | 8.29 (d, J = 1.2 Hz, 1H), 8.19-8.17 (m, 1H), 7.58 |
| | | | | (dd, J = 2.0, 13.6 Hz, 3H), 7.08 (d, J = 7.2 Hz, |
| | | | | 1H), 6.93 (d, J = 8.4 Hz, 1H), 6.57-6.45 (m, 1H), |
| | | | | 5.10-5.00 (m, 1H), 4.51-4.39 (m, 1H), 4.22- |
| | | | | 4.09 (m, 2H), 2.97-2.79 (m, 1H), 2.71-2.65 (m, |
| | | | | 1H), 2.63-2.55 (m, 1H), 2.53 (s, 3H), 2.41-2.35 |
| | | | | (m, 5H), 2.35-2.31 (m, 2H), 2.29-2.21 (m, 3H), |
| | | | | 2.05-1.86 (m, 6H), 1.74-1.53 (m, 4H), 1.19- |
| | | | | 1.06 (m, 2H) |
| I-355 | CBX | BRP | 771.3 | 11.10 (s, 1H), 10.37 (s, 1H), 8.44-8.30 (m, 4H), |
| | | | | 8.18 (dd, J = 1.2, 7.6 Hz, 1H), 7.65-7.60 (m, 1H), |
| | | | | 7.59-7.53 (m, 1H), 7.05-6.95 (m, 1H), 6.72 (d, |
| | | | | J = 7.6 Hz, 1H), 5.65 (t, J = 8.0 Hz, 1H), 5.36 (dd, |
| | | | | J = 5.2, 12.4 Hz, 1H), 4.58-4.43 (m, 1H), 3.75 (s, |
| | | | | 3H), 3.73 (s, 1H), 3.34-3.33 (m, 3H), 3.30 (s, |
| | | | | 1H), 3.26 (s, 1H), 3.18-3.06 (m, 1H), 2.98-2.83 |
| | | | | (m, 2H), 2.77-2.69 (m, 1H), 2.63 (d, J = 17.2 Hz, |
| | | | | 2H), 2.53 (s, 1H), 2.21 (d, J = 10.3 Hz, 3H), 2.08- |
| | | | | 1.93 (m, 6H), 1.39-1.16 (m, 2H) |
| I-356 | CCF | ATJ | 770.3 | 10.49 (d, J = 6.4 Hz, 2H), 9.02 (s, 1H), 8.69 (s, |
| | | | | 1H), 8.48-8.44 (m, 1H), 8.43-8.37 (m, 1H), 8.31 |
| | | | | (s, 1H), 8.21 (d, J = 7.6 Hz, 1H), 8.17 (s, 1H), 7.77 |
| | | | | (d, J = 9.2 Hz, 1H), 7.50 (dd, J = 2.0, 9.2 Hz, 1H), |
| | | | | 7.22 (s, 1H), 7.14 (s, 1H), 4.36 (t, J = 11.6 Hz, |
| | | | | 1H), 3.98 (s, 3H), 3.92-3.73 (m, 3H), 3.72-3.66 |
| | | | | (m, 1H), 3.63 (t, J = 6.4 Hz, 2H), 3.13-2.80 (m, |
| | | | | 4H), 2.78-2.70 (m, 2H), 2.52 (s, 1H), 2.11 (s, |
| | | | | 4H), 1.99-1.39 (m, 6H), 1.26-1.00 (m, 2H) |
| I-357 | CCG | ATJ | 753.3 | 10.56 (s, 1H), 10.51 (s, 1H), 9.57-9.30 (m, 1H), |
| | | | | 8.70 (s, 1H), 8.59 (s, 1H), 8.49-8.44 (m, 1H), |
| | | | | 8.44-8.38 (m, 1H), 8.36 (s, 1H), 8.22 (d, J = 7.6 |
| | | | | Hz, 1H), 8.06-7.88 (m, 1H), 7.88-7.74 (m, 1H), |
| | | | | 7.58 (dd, J = 1.6, 4.8 Hz, 1H), 7.16 (s, 1H), 5.88 |
| | | | | (s, 1H), 4.52-4.37 (m, 1H), 4.34 (t, J = 5.2 Hz, |
| | | | | 1H), 3.99 (s, 3H), 3.98-3.85 (m, 2H), 3.73 (td, J = |
| | | | | 6.0, 12.0 Hz, 1H), 3.44 (t, J = 2.0 Hz, 1H), 3.26- |
| | | | | 3.11 (m, 2H), 3.10-2.92 (m, 2H), 2.77 (d, J = |
| | | | | 5.2 Hz, 2H), 2.21 (d, J = 2.0 Hz, 2H), 2.12-1.84 |
| | | | | (m, 5H), 1.46-1.07 (m, 3H) |
| I-358 | BXL | CCH | 760.6 | 10.57-10.49 (m, 2H), 9.52-9.24 (m, 1H), 8.65 |
| | | | | (s, 1H), 8.60-8.51 (m, 2H), 8.49-8.38 (m, 2H), |
| | | | | 8.25-8.23 (m, 1H), 7.94 (s, 1H), 7.80-7.60 (m, |
| | | | | 2H), 7.41-7.25 (m, 1H), 4.60-4.46 (m, 1H), 3.98- |
| | | | | 3.85 (m, 1H), 3.73-3.67 (m, 2H), 3.62-3.44 |
| | | | | (m, 2H), 3.28-3.08 (m, 4H), 3.01-2.92 (m, 1H), |
| | | | | 2.79-2.72 (m, 2H), 2.53-2.51 (m, 1H), 2.22- |
| | | | | 2.20 (m, 2H), 2.04-2.01 (m, 5H), 1.88-1.59 (m, |
| | | | | 1H), 1.40-1.14 (m, 2H) |
| I-359 | BAI | BKQ | 762.6 | 11.10 (s, 1H), 10.23 (d, J = 2.0 Hz, 1H), 8.50 (s, |
| | | | | 1H), 8.46-8.38 (m, 3H), 8.22 (d, J = 7.6 Hz, 1H), |
| | | | | 7.55 (d, J = 11.6 Hz, 1H), 7.08-6.86 (m, 3H), 5.40- |
| | | | | 5.31 (m, 1H), 4.55-4.25 (m, 1H), 3.64 (s, 3H), |
| | | | | 3.31-3.23 (m, 3H), 3.22-3.13 (m, 2H), 3.04- |
| | | | | 2.97 (m, 1H), 2.94-2.84 (m, 2H), 2.76-2.59 (m, |
| | | | | 3H), 2.29-2.15 (m, 3H), 2.03-1.89 (m, 6H), 1.87- |
| | | | | 1.57 (m, 1H), 1.40-1.07 (m, 2H) |
| I-360 | BAI | CCH | 778.6 | 11.10 (s, 1H), 10.53 (s, 1H), 8.64 (s, 1H), 8.54 (s, |
| | | | | 1H), 8.50-8.37 (m, 2H), 8.24 (d, J = 7.6 Hz, 1H), |
| | | | | 7.94 (s, 1H), 7.09-6.86 (m, 3H), 5.37 (dd, J = 5.2, |
| | | | | 12.4 Hz, 1H), 4.52 (s, 1H), 3.65 (s, 4H), 3.26- |
| | | | | 3.06 (m, 4H), 3.04-2.80 (m, 3H), 2.78-2.55 (m, |
| | | | | 3H), 2.29-2.13 (m, 3H), 2.08-1.89 (m, 6H), 1.88- |
| | | | | 1.49 (m, 1H), 1.40-1.10 (m, 2H) |
| I-361 | BAI | BUR | 758.4 | 11.09 (s, 1H), 8.81 (s, 1H), 8.67 (s, 1H), 8.58 (s, |
| | | | | 2H), 8.52 (d, J = 2.0 Hz, 1H), 8.42-8.27 (m, 1H), |
| | | | | 8.08 (s, 1H), 7.14-6.80 (m, 4H), 5.43-5.27 (m, |
| | | | | 1H), 3.83-3.70 (m, 2H), 3.63 (s, 3H), 3.61-3.52 |
| | | | | (m, 1H), 3.22-3.07 (m, 3H), 3.05-2.77 (m, 4H), |
| | | | | 2.76-2.68 (m, 1H), 2.65-2.58 (m, 1H), 2.29- |
| | | | | 2.09 (m, 2H), 2.05-1.71 (m, 6H), 1.53-1.34 (m, |
| | | | | 2H), 1.29 (d, J = 5.6 Hz, 6H), 1.24-0.86 (m, 3H) |
| I-362 | BAI | BVU | 758.3 | 11.09 (s, 1H), 8.83 (d, J = 2.0 Hz, 1H), 8.71 (d, J = |
| | | | | 2.0 Hz, 1H), 8.68 (s, 1H), 8.46 (d, J = 7.2 Hz, |
| | | | | 1H), 8.30 (d, J = 7.6 Hz, 1H), 8.05 (s, 1H), 7.82 |
| | | | | (d, J = 4.8 Hz, 1H), 7.10 (d, J = 4.8 Hz, 1H), 7.01- |
| | | | | 6.92 (m, 2H), 6.88 (d, J = 7.6 Hz, 1H), 5.38- |
| | | | | 5.31 (m, 1H), 3.81-3.71 (m, 2H), 3.63 (s, 3H), |
| | | | | 3.02-2.80 (m, 8H), 2.72-2.69 (m, 1H), 2.19 (d, |
| | | | | J = 7.2 Hz, 3H), 2.03-1.96 (m, 1H), 1.91-1.82 |
| | | | | (m, 4H), 1.55-1.46 (m, 1H), 1.43-1.30 (m, 3H), |
| | | | | 1.27 (d, J = 6.4 Hz, 6H), 1.03-0.92 (m, 2H) |
| I-363 | BAI | BXI | 769.4 | 11.09 (s, 1H), 10.64 (s, 1H), 8.63 (s, 1H), 8.52- |
| | | | | 8.31 (m, 3H), 8.28-8.19 (m, 2H), 7.03-6.88 (m, |
| | | | | 3H), 5.36 (dd, J = 5.2, 12.8 Hz, 1H), 4.67-4.50 |
| | | | | (m, 1H), 3.64 (s, 3H), 3.12-2.97 (m, 3H), 2.95- |
| | | | | 2.81 (m, 3H), 2.64 (dd, J = 12.8, 17.2 Hz, 4H), |
| | | | | 2.31-2.25 (m, 1H), 2.20 (d, J = 9.6 Hz, 3H), 2.10- |
| | | | | 1.86 (m, 6H), 1.81-1.63 (m, 1H), 1.32-1.12 (m, 2H) |
| I-364 | CCS | ATJ | 804.6 | 11.10 (s, 1H), 10.53 (s, 1H), 8.64 (s, 1H), 8.54 (s, |
| | | | | 1H), 8.50-8.37 (m, 2H), 8.24 (d, J = 7.6 Hz, 1H), |
| | | | | 7.94 (s, 1H), 7.09-6.86 (m, 3H), 5.37 (dd, J = 5.2, |
| | | | | 12.4 Hz, 1H), 4.52 (s, 1H), 3.65 (s, 4H), 3.26- |
| | | | | 3.06 (m, 4H), 3.04-2.80 (m, 3H), 2.78-2.55 (m, |
| | | | | 3H), 2.29-2.13 (m, 3H), 2.08-1.89 (m, 6H), 1.88- |
| | | | | 1.49 (m, 1H), 1.40-1.10 (m, 2H) |
| I-365 | BAI | CFG | 720.6 | 11.08 (s, 1H), 9.60 (s, 1H), 8.92 (s, 1H), 8.60 (s, |
| | | | | 1H), 8.29 (s, 1H), 8.13 (s, 1H), 8.07 (s, 1H), 7.07 |
| | | | | (s, 1H), 7.02-6.92 (m, 2H), 6.89 (d, J = 7.6 Hz, |
| | | | | 1H), 5.40-5.31 (m, 1H), 4.44-4.33 (m, 1H), 3.87 |
| | | | | (s, 3H), 3.64 (s, 3H), 3.30 (s, 1H), 3.10-2.79 (m, |
| | | | | 7H), 2.76-2.62 (m, 2H), 2.52 (s, 1H), 2.39 (s, |
| | | | | 3H), 2.27-2.23 (m, 2H), 2.20-2.13 (m, 2H), 2.02- |
| | | | | 1.86 (m, 5H), 1.74-1.60 (m, 1H), 1.22-1.07 (m, 2H) |
| I-366 | CFH | ATJ | 757.5 | 11.12 (s, 1H), 10.51 (s, 1H), 8.70 (s, 1H), 8.48- |
| | | | | 8.38 (m, 2H), 8.35 (s, 1H), 8.22 (d, J = 7.6 Hz, |
| | | | | 1H), 7.39 (s, 1H), 7.22-7.11 (m, 3H), 6.37 (s, |
| | | | | 1H), 5.39 (dd, J = 5.2, 12.4 Hz, 1H), 4.54-4.31 |
| | | | | (m, 2H), 3.99 (s, 3H), 3.38 (s, 3H), 3.26-3.02 (m, |
| | | | | 2H), 3.01-2.82 (m, 2H), 2.80-2.61 (m, 3H), 2.19 |
| | | | | (d, J = 12.0 Hz, 2H), 2.12-1.88 (m, 6H), 1.88- |
| | | | | 1.70 (m, 1H), 1.37-1.16 (m, 2H) |
| I-367 | CAR | BVB | 745.4 | 11.10 (s, 1H), 9.92 (s, 1H), 9.32 (d, J = 1.6 Hz, |
| | | | | 1H), 9.22 (d, J = 1.6 Hz, 1H), 8.79 (s, 1H), 8.33 |
| | | | | (s, 1H), 8.12 (d, J = 19.6 Hz, 1H), 7.08 (s, 1H), |
| | | | | 7.03-6.97 (m, 2H), 6.92 (d, J = 5.2 Hz, 1H), 5.37 |
| | | | | (dd, J = 5.6, 12.4 Hz, 1H), 4.48-4.35 (m, 1H), |
| | | | | 3.89 (s, 3H), 3.66 (s, 3H), 3.31-3.30 (m, 1H), |
| | | | | 3.22-3.06 (m, 4H), 2.96-2.83 (m, 2H), 2.78- |
| | | | | 2.69 (m, 1H), 2.69-2.58 (m, 2H), 2.53 (d, J = 2.0 |
| | | | | Hz, 2H), 2.19 (d, J = 12.0 Hz, 2H), 2.12-1.85 (m, |
| | | | | 8H), 1.82-1.68 (m, 1H), 1.31-1.13 (m, 2H) |
| I-368 | AZK | CFI | 800.4 | 11.09 (s, 1H), 8.77 (s, 1H), 8.60-8.52 (m, 4H), |
| | | | | 8.33 (d, J = 7.6 Hz, 1H), 8.19 (s, 1H), 7.04-6.94 |
| | | | | (m, 3H), 6.91 (d, J = 4.0 Hz, 1H), 5.39-5.35 (m, |
| | | | | 1H), 3.86-3.69 (m, 2H), 3.59 (s, 3H), 3.13-3.01 |
| | | | | (m, 2H), 2.96-2.82 (m, 1H), 2.77-2.68 (m, 1H), |
| | | | | 2.64-2.59 (m, 1H), 2.52-2.51 (m, 4H), 2.03-1.96 |
| | | | | (m, 1H), 1.95-1.74 (m, 9H), 1.61-1.50 (m, 1H), 1.38- |
| | | | | 1.35 (m, 2H), 1.30-1.29 (m, 6H), 1.06-0.93 (m, 2H) |
| I-369 | BAI | CFI | 801.7 | 11.09 (s, 1H), 8.77 (d, J = 1.6 Hz, 1H), 8.61-8.49 |
| | | | | (m, 4H), 8.39-8.30 (m, 1H), 8.19 (s, 1H), 7.05- |
| | | | | 6.85 (m, 4H), 5.38-5.34 (m, 1H), 3.85-3.71 (m, |
| | | | | 2H), 3.64 (s, 3H), 3.21-3.03 (m, 3H), 2.94-2.83 |
| | | | | (m, 2H), 2.76-2.68 (m, 1H), 2.66-2.58 (m, 1H), |
| | | | | 2.52-2.51 (m, 7H), 2.04-1.96 (m, 1H), 1.95- |
| | | | | 1.84 (m, 4H), 1.47-1.34 (m, 2H), 1.30-0.29 (m, |
| | | | | 6H), 1.17-0.95 (m, 2H) |
| I-370 | BSN | CCH | 759.2 | 9.74-9.69 (m, 2H), 8.84 (s, 1H), 7.82 (s, 1H), |
| | | | | 7.78-7.74 (m, 1H), 7.72 (s, 1H), 7.67-7.63 (m, |
| | | | | 1H), 7.62-7.57 (m, 1H), 7.42 (d, J = 7.6 Hz, 1H), |
| | | | | 7.13-7.10 (m, 1H), 7.09-7.05 (m, 1H), 6.99 (t, J = |
| | | | | 7.6 Hz, 1H), 6.82-6.80 (m, 1H), 3.76-3.65 (m, |
| | | | | 1H), 3.17-3.06 (m, 1H), 2.94-2.85 (m, 1H), 2.56- |
| | | | | 2.51 (m, 3H), 2.20-2.12 (m, 1H), 1.98-1.91 |
| | | | | (m, 2H), 1.70-1.69 (m, 4H), 1.40-1.37 (m, 2H), |
| | | | | 1.28-1.11 (m, 7H), 0.49-0.34 (m, 2H) |
| I-371 | BXL | ALU | 776.1 | 10.55 (s, 1H), 10.50 (s, 1H), 9.54-9.26 (m, 1H), |
| | | | | 8.58 (s, 2H), 8.50 (s, 1H), 8.48-8.44 (m, 1H), |
| | | | | 8.43-8.37 (m, 1H), 8.23 (d, J = 7.6 Hz, 1H), 8.00 |
| | | | | (s, 1H), 7.85-7.59 (m, 2H), 7.42-7.07 (m, 2H), |
| | | | | 4.69-4.47 (m, 1H), 4.01-3.84 (m, 1H), 3.79- |
| | | | | 3.65 (m, 2H), 3.65-3.42 (m, 3H), 3.31-3.07 (m, |
| | | | | 6H), 2.98 (d, J = 6.0 Hz, 1H), 2.76 (d, J = 5.6 Hz, |
| | | | | 2H), 2.29-2.18 (m, 2H), 2.04 (d, J = 7.6 Hz, 4H), |
| | | | | 1.47-1.11 (m, 2H) |
| I-372 | BXL | BUR | 740.4 | 10.54 (s, 1H), 9.46 (s, 1H), 8.81 (d, J = 2.0 Hz, |
| | | | | 1H), 8.68 (d, J = 2.0 Hz, 1H), 8.60-8.54 (m, 3H), |
| | | | | 8.52 (d, J = 4.0 Hz, 1H), 8.37 (d, J = 7.6 Hz, 1H), |
| | | | | 8.08 (s, 1H), 7.80-7.60 (m, 2H), 7.32 (s, 1H), |
| | | | | 6.89 (d, J = 4.0 Hz, 1H), 3.91 (ddd, J = 5.2, 9.6, |
| | | | | 12.0 Hz, 1H), 3.83-3.65 (m, 4H), 3.40 (s, 1H), |
| | | | | 3.25-3.05 (m, 4H), 2.97 (ddd, J = 6.0, 10.0, 16.4 |
| | | | | Hz, 2H), 2.76 (td, J = 5.6, 16.8 Hz, 2H), 2.52 (d, J = |
| | | | | 2.0 Hz, 2H), 1.91 (d, J = 8.0 Hz, 4H), 1.48-1.36 |
| | | | | (m, 2H), 1.29 (d, J = 6.4 Hz, 6H), 1.21-1.12 (m, |
| | | | | 1H), 1.05 (t, J = 7.2 Hz, 2H) |
| I-373 | BXL | BVU | 740.4 | 10.54 (s, 1H), 9.51-9.43 (m, 1H), 8.84 (d, J = 2.0 |
| | | | | Hz, 1H), 8.73-8.67 (m, 2H), 8.56 (s, 1H), 8.51- |
| | | | | 8.43 (m, 1H), 8.34 (d, J = 6.0 Hz, 1H), 8.05 (s, |
| | | | | 1H), 7.83 (d, J = 4.8 Hz, 1H), 7.78-7.64 (m, 2H), |
| | | | | 7.40-7.26 (m, 1H), 7.11 (d, J = 4.8 Hz, 1H), 3.96- |
| | | | | 3.87 (m, 1H), 3.83-3.75 (m, 2H), 3.73-3.65 |
| | | | | (m, 2H), 3.58-3.44 (m, 2H), 3.21-3.08 (m, 3H), |
| | | | | 3.03-2.91 (m, 2H), 2.81-2.70 (m, 2H), 2.52 (d, J = |
| | | | | 2.0 Hz, 2H), 1.91 (d, J = 8.4 Hz, 5H), 1.47-1.34 |
| | | | | (m, 2H), 1.28 (d, J = 6.4 Hz, 6H), 1.19-1.05 (m, 2H) |
| I-374 | CAY | CFI | 801.2 | 11.07 (s, 1H), 8.77 (d, J = 1.6 Hz, 1H), 8.61-8.55 |
| | | | | (m, 3H), 8.54 (d, J = 4.0 Hz, 1H), 8.35 (d, J = 7.6 |
| | | | | Hz, 1H), 8.19 (s, 1H), 7.02-6.93 (m, 1H), 6.91 (d, |
| | | | | J = 4.0 Hz, 2H), 6.73-6.59 (m, 1H), 5.30 (dd, J = |
| | | | | 4.8, 12.8 Hz, 1H), 3.86-3.67 (m, 3H), 3.67-3.48 |
| | | | | (m, 1H), 3.33-3.33 (m, 3H), 3.24-2.98 (m, 5H), |
| | | | | 2.96-2.84 (m, 1H), 2.75-2.57 (m, 3H), 2.52 (d, |
| | | | | J = 2.0 Hz, 1H), 2.26-2.08 (m, 1H), 2.04-1.66 |
| | | | | (m, 6H), 1.46-1.33 (m, 2H), 1.30 (d, J = 6.4 Hz, |
| | | | | 6H), 1.17-0.89 (m, 2H) |
| I-375 | BWL | ALU | 776.3 | 10.54-10.46 (m, 2H), 9.08 (s, 1H), 8.57 (s, 1H), |
| | | | | 8.49 (s, 1H), 8.48-8.44 (m, 1H), 8.43-8.36 (m, |
| | | | | 1H), 8.28 (s, 1H), 8.23 (dd, J = 0.4, 7.2 Hz, 1H), |
| | | | | 7.99 (s, 1H), 7.81 (d, J = 9.2 Hz, 1H), 7.68 (dd, J = |
| | | | | 2.4, 9.6 Hz, 1H), 7.42 (d, J = 2.0 Hz, 1H), 7.39- |
| | | | | 7.09 (m, 1H), 4.61-4.48 (m, 1H), 3.97-3.84 |
| | | | | (m, 1H), 3.76-3.62 (m, 1H), 3.40-3.34 (m, 4H), |
| | | | | 3.00-2.88 (m, 1H), 2.80-2.70 (m, 1H), 2.63-2.56 |
| | | | | (m, 4H), 2.31-2.22 (m, 2H), 2.23-2.12 (m, 2H), 2.06- |
| | | | | 1.91 (m, 4H), 1.80-1.65 (m, 1H), 1.27-1.10 (m, 2H) |
| I-376 | CFK | ATJ | 786.6 | 10.53 (d, J = 14.4 Hz, 2H), 9.40 (s, 1H), 9.28- |
| | | | | 9.16 (m, 1H), 8.71 (s, 1H), 8.47-8.45 (m, 1H), |
| | | | | 8.44-8.38 (m, 1H), 8.35 (s, 1H), 8.22 (d, J = 7.6 |
| | | | | Hz, 1H), 7.78-7.71 (m, 1H), 7.70-7.63 (m, 1H), |
| | | | | 7.15 (s, 1H), 4.45 (t, J = 11.2 Hz, 1H), 4.02 (s, |
| | | | | 3H), 3.99 (s, 3H), 3.92 (ddd, J = 5.2, 9.6, 12.0 Hz, |
| | | | | 1H), 3.79 (d, J = 12.4 Hz, 2H), 3.75-3.66 (m, |
| | | | | 3H), 3.27-3.17 (m, 5H), 3.15-3.06 (m, 1H), 2.99- |
| | | | | 2.88 (m, 1H), 2.85-2.73 (m, 1H), 2.21 (d, J = |
| | | | | 10.4 Hz, 2H), 2.05-1.92 (m, 5H), 1.39-1.23 (m, 2H) |
| I-377 | BXL | CFL | 754.4 | 10.53 (s, 1H), 10.18 (s, 1H), 9.44 (s, 1H), 8.54 (s, |
| | | | | 1H), 8.46-8.42 (m, 1H), 8.40 (d, J = 7.6 Hz, 1H), |
| | | | | 8.39-8.36 (m, 1H), 8.25 (s, 1H), 8.21 (d, J = 1.2 |
| | | | | Hz, 1H), 7.78-7.70 (m, 1H), 7.67-7.59 (m, 1H), |
| | | | | 7.53-7.47 (m, 1H), 7.34-7.26 (m, 1H), 4.53- |
| | | | | 4.41 (m, 1H), 3.91 (d, J = 12.0 Hz, 1H), 3.75-3.65 |
| | | | | (m, 1H), 3.26-3.08 (m, 4H), 2.97 (d, J = 6.0 Hz, |
| | | | | 2H), 2.82-2.69 (m, 6H), 2.23-2.13 (m, 2H), 2.07- |
| | | | | 1.88 (m, 5H), 1.81-1.65 (m, 1H), 1.31-1.12 (m, 6H) |
| I-378 | BAI | CFL | 772.2 | 11.10 (s, 1H), 10.18 (s, 1H), 8.51-8.42 (m, 1H), |
| | | | | 8.40 (d, J = 7.6 Hz, 1H), 8.38 (s, 1H), 8.25 (s, 1H), |
| | | | | 8.22 (d, J = 1.2 Hz, 1H), 7.50 (s, 1H), 7.07-6.99 |
| | | | | (m, 1H), 6.99-6.88 (m, 2H), 5.37 (d, J = 5.2 Hz, |
| | | | | 1H), 4.55-4.41 (m, 1H), 3.65 (s, 4H), 3.29-3.02 |
| | | | | (m, 5H), 2.97-2.84 (m, 2H), 2.83-2.70 (m, 3H), |
| | | | | 2.70-2.60 (m, 2H), 2.27-2.12 (m, 3H), 2.06- |
| | | | | 1.86 (m, 6H), 1.26 (t, J = 7.6 Hz, 6H) |
| I-379 | BAI | CFM | 829.7 | 11.14-11.05 (m, 2H), 8.79 (s, 1H), 8.52-8.47 (m, |
| | | | | 1H), 8.44-8.37 (m, 2H), 8.29-8.21 (m, 1H), 7.51 |
| | | | | (s, 1H), 7.08-6.86 (m, 3H), 5.42-5.33 (m, 12.4 |
| | | | | Hz, 1H), 4.51-4.38 (m, 1H), 3.87 (s, 4H), 3.65 (s, |
| | | | | 3H), 3.25-3.05 (m, 4H), 2.92 (s, 5H), 2.77-2.59 |
| | | | | (m, 5H), 2.54 (s, 2H), 2.21-2.14 (m, 2H), 2.06- |
| | | | | 1.88 (m, 6H), 1.44-1.03 (m, 3H) |
| I-380 | CFO | ATJ | 757.6 | 10.78 (s, 1H), 10.51 (s, 1H), 9.17 (s, 1H), 8.70 (s, |
| | | | | 1H), 8.48-8.44 (m, 1H), 8.44-8.38 (m, 1H), 8.35 |
| | | | | (s, 1H), 8.23-8.21 (m, 1H), 7.69 (d, J = 7.6 Hz, |
| | | | | 1H), 7.61-7.57 (m, J = 7.6 Hz, 1H), 7.44 (d, J = |
| | | | | 6.4 Hz, 1H), 7.16 (s, 1H), 4.49-4.39 (m, 1H), 4.15- |
| | | | | 4.01 (m, 3H), 3.99 (s, 3H), 3.80-3.66 (m, 2H), |
| | | | | 3.47-3.37 (m, 2H), 3.27-3.14 (m, 3H), 2.85- |
| | | | | 2.84 (m, 2H), 2.52-2.50 (m, 2H), 2.21-2.18 (m, |
| | | | | 2H), 2.08-1.89 (m, 5H), 1.32-1.29 (m, 2H) |
| I-381 | CGV | AJB | 865.5 | 11.12 (s, 1H), 9.50 (d, J = 6.4 Hz, 1H), 8.78 (d, J = |
| | | | | 7.6 Hz, 1H), 8.39 (s, 1H), 8.25 (d, J = 5.6 Hz, |
| | | | | 1H), 7.25-6.95 (m, 4H), 6.91-6.41 (m, 1H), 5.40 |
| | | | | (dd, J = 5.2, 12.4 Hz, 1H), 5.30-5.03 (m, 1H), |
| | | | | 4.82-4.70 (m, 1H), 4.30-4.12 (m, 1H), 3.86- |
| | | | | 3.71 (m, 3H), 3.66 (s, 3H), 3.63-3.42 (m, 3H), |
| | | | | 3.04-2.96 (m, 1H), 2.93-2.83 (m, 2H), 2.78- |
| | | | | 2.68 (m, 2H), 2.68-2.59 (m, 2H), 2.52-2.51 (m, |
| | | | | 2H), 2.24-1.86 (m, 9H), 1.85-1.47 (m, 4H), 1.31 |
| | | | | (s, 2H), 1.20-0.92 (m, 3H) |
| I-382 | CFQ | ATJ | 756.2 | 10.59 (s, 1H), 10.51 (s, 1H), 8.93 (d, J = 4.4 Hz, |
| | | | | 1H), 8.70 (s, 1H), 8.49-8.44 (m, 1H), 8.43-8.37 |
| | | | | (m, 1H), 8.35 (s, 1H), 8.22 (d, J = 8.4 Hz, 1H), |
| | | | | 7.70-7.50 (m, 3H), 7.27 (s, 1H), 7.16 (s, 1H), |
| | | | | 4.48-4.38 (m, 1H), 4.29-4.09 (m, 1H), 4.07- |
| | | | | 3.91 (m, 5H), 3.87-3.59 (m, 3H), 3.54-3.34 (m, |
| | | | | 3H), 3.29-3.15 (m, 3H), 3.06-2.96 (m, 1H), 2.75- |
| | | | | 2.68 (m, 1H), 2.19 (d, J = 8.0 Hz, 2H), 2.09- |
| | | | | 1.88 (m, 5H), 1.42-1.18 (m, 2H) |
| I-383 | BAI | BAS | 841.4 | 11.08 (s, 1H), 10.69 (s, 1H), 8.68 (s, 1H), 8.45- |
| | | | | 8.53 (m, 1H), 8.40 (t, J = 7.6 Hz, 1H), 8.34 (s, 1H), |
| | | | | 8.26-8.16(m, 1H), 7.39 (s, 1H), 6.92-7.03 (m, |
| | | | | 2H), 6.89 (d, J = 7.6 Hz, 1H), 5.35 (dd, J = 12.8, 5.2 |
| | | | | Hz, 1H), 4.61 (s, 1H), 4.32-4.47 (m, 1H), 4.05 (s, |
| | | | | 1H), 4.00 (d, J = 7.6 Hz, 1H), 3.66 (dd, J = 7.6, 1.6 |
| | | | | Hz, 1H), 3.64 (s, 3H), 3.42 (d, J = 10.0 Hz, 1H), |
| | | | | 3.24 (d, J = 9.2 Hz, 1H), 2.84-3.01 (m, 6H), 2.56- |
| | | | | 2.77 (m, 3H), 2.24-2.35 (m, 3H), 2.13 (dd, |
| | | | | J = 17.6, 10.8 Hz, 3H), 1.82-2.05 (m, 7H), 1.61- |
| | | | | 1.74 (m, 1H), 1.10-1.24 (m, 2H) |
| I-384 | BAI | BQY | 841.4 | 11.10 (s, 1H), 10.69 (s, 1H), 8.69 (s, 1H), 8.53- |
| | | | | 8.45 (m, 1H), 8.41 (t, J = 7.6 Hz, 1H), 8.35 (s, 1H), |
| | | | | 8.22 (d, J = 7.6 Hz, 1H), 7.38 (s, 1H), 7.12-6.81 |
| | | | | (m, 3H), 5.47-5.29 (m, 1H), 4.62 (s, 1H), 4.54- |
| | | | | 4.35 (m, 1H), 4.05 (s, 1H), 4.00 (d, J = 7.6 Hz, |
| | | | | 1H), 3.71-3.58 (m, 5H), 3.43 (d, J = 10.0 Hz, |
| | | | | 1H), 3.29-3.22 (m, 3H), 3.21-3.10 (m, 3H), 2.95- |
| | | | | 2.84 (m, 2H), 2.77-2.69 (m, 1H), 2.69-2.62 |
| | | | | (m, 2H), 2.62-2.57 (m, 1H), 2.29-2.14 (m, 3H), |
| | | | | 2.11 (d, J = 9.2 Hz, 1H), 2.06-1.90 (m, 6H), 1.85 |
| | | | | (d, J = 9.2 Hz, 1H), 1.41-1.06 (m, 2H) |
| I-385 | CFR | ATJ | 843.6 | 11.09 (s, 1H), 10.50 (s, 1H), 8.69 (d, J = 0.8 Hz, |
| | | | | 1H), 8.52-8.36 (m, 2H), 8.33 (s, 1H), 8.22 (d, J = |
| | | | | 7.6 Hz, 1H), 7.15 (s, 1H), 7.11-6.92 (m, 3H), |
| | | | | 5.36 (dd, J = 4.8, 12.0 Hz, 1H), 4.37 (d, J = 14.8 |
| | | | | Hz, 2H), 4.16-4.05 (m, 1H), 3.98 (s, 3H), 3.57 (s, |
| | | | | 4H), 3.43 (d, J = 4.0 Hz, 1H), 3.14-3.07 (m, 1H), |
| | | | | 2.96-2.83 (m, 2H), 2.74-2.59 (m, 5H), 2.22- |
| | | | | 2.05 (m, 4H), 2.03-1.88 (m, 7H), 1.83-1.55 (m, |
| | | | | 3H), 1.26-1.13 (m, 2H), 1.06 (m, 2H) |
| I-386 | CFS | ATJ | 843.2 | 11.09 (s, 1H), 10.50 (s, 1H), 8.69 (s, 1H), 8.50- |
| | | | | 8.36 (m, 2H), 8.33 (s, 1H), 8.22 (d, J = 7.6 Hz, |
| | | | | 1H), 7.22-7.09 (m, 2H), 7.08-6.94 (m, 2H), 5.37 |
| | | | | (dd, J = 4.8, 12.0 Hz, 1H), 4.46-4.31 (m, 2H), |
| | | | | 4.30-4.21 (m, 1H), 4.08 (s, 1H), 3.98 (s, 3H), |
| | | | | 3.51 (s, 4H), 3.43 (d, J = 5.2 Hz, 1H), 3.11 (s, 1H), |
| | | | | 2.96-2.83 (m, 2H), 2.81-2.58 (m, 5H), 2.16 (d, |
| | | | | J = 8.4 Hz, 2H), 2.09-1.85 (m, 8H), 1.84-1.62 |
| | | | | (m, 3H), 1.28-1.12 (m, 2H), 1.11-1.01 (m, 2H) |
| I-387 | BAI | CFT | 799.3 | 11.10 (s, 1H), 10.19 (s, 1H), 8.49-8.36 (m, 3H), |
| | | | | 8.29 (s, 1H), 8.23 (d, J = 7.6 Hz, 1H), 7.10-6.92 |
| | | | | (m, 3H), 6.91 (s, 1H), 5.47-5.28 (m, 1H), 4.54- |
| | | | | 4.29 (m, 1H), 3.88 (t, J = 7.2 Hz, 4H), 3.65 (s, 3H), |
| | | | | 3.64-3.54 (m, 1H), 3.44-3.34 (m, 2H), 3.17 (s, |
| | | | | 4H), 3.06-2.78 (m, 3H), 2.77-2.63 (m, 2H), 2.31- |
| | | | | 2.21 (m, 3H), 2.18 (d, J = 4.4 Hz, 2H), 2.09- |
| | | | | 1.80 (m, 6H), 1.40-1.06 (m, 2H) |
| I-388 | BAI | BUE | 787.5 | 11.10 (s, 1H), 11.05 (s, 1H), 8.73 (s, 1H), 8.49- |
| | | | | 8.45 (m, 1H), 8.44-8.38 (m, 1H), 8.36 (s, 1H), |
| | | | | 8.21 (d, J = 7.6 Hz, 1H), 7.44 (s, 1H), 7.11-7.00 |
| | | | | (m, 1H), 7.00-6.84 (m, 2H), 5.48-5.26 (m, 1H), |
| | | | | 4.55-4.34 (m, 1H), 3.65 (s, 4H), 3.40-3.00 (m, |
| | | | | 6H), 2.98-2.81 (m, 2H), 2.80-2.70 (m, 6H), 2.73- |
| | | | | 2.68 (m, 1H), 2.65-2.58 (m, 1H), 2.47-2.41 |
| | | | | (m, 1H), 2.29-2.09 (m, 3H), 2.06-1.85 (m, 6H), |
| | | | | 1.39-1.18 (m, 2H) |
| I-389 | BDY | BSC | 770.2 | 11.10 (s, 1H), 10.13 (s, 1H), 8.47-8.33 (m, 4H), |
| | | | | 8.25-8.16 (m, 2H), 7.56-7.47 (m, 1H), 7.14- |
| | | | | 6.84 (m, 4H), 5.36 (d, J = 5.2 Hz, 1H), 4.52-4.41 |
| | | | | (m, 2H), 4.15-3.96 (m, 1H), 3.85 (d, J = 3.2 Hz, |
| | | | | 1H), 3.74-3.58 (m, 5H), 2.98-2.82 (m, 2H), 2.77- |
| | | | | 2.56 (m, 4H), 2.20 (d, J = 10.8 Hz, 3H), 2.10- |
| | | | | 1.88 (m, 7H), 1.78-1.67 (m, 1H), 1.36-1.19 (m, 2H) |
| I-391 | CCS | BUR | 788.3 | 11.08 (s, 1H), 8.81 (d, J = 2.0 Hz, 1H), 8.68 (d, J = |
| | | | | 2.0 Hz, 1H), 8.58 (s, 2H), 8.52 (d, J = 3.6 Hz, |
| | | | | 1H), 8.36 (d, J = 7.2 Hz, 1H), 8.08 (s, 1H), 6.92 |
| | | | | (d, J = 8.4 Hz, 1H), 6.89 (d, J = 4.0 Hz, 1H), 6.72 |
| | | | | (d, J = 8.8 Hz, 1H), 5.32 (dd, J = 5.2, 12.4 Hz, |
| | | | | 1H), 3.81 (s, 3H), 3.79-3.73 (m, 2H), 3.61 (s, |
| | | | | 5H), 3.14-3.06 (m, 1H), 3.04-2.93 (m, 2H), 2.92- |
| | | | | 2.81 (m, 2H), 2.73-2.57 (m, 4H), 2.01-1.81 |
| | | | | (m, 6H), 1.80-1.54 (m, 1H), 1.39 (d, J = 11.2 Hz, |
| | | | | 2H), 1.29 (d, J = 6.4 Hz, 6H), 1.24-1.14 (m, 1H), |
| | | | | 1.13-1.00 (m, 2H) |
| I-392 | BAI | CFU | 813.3 | 11.10 (s, 1H), 10.89 (s, 1H), 8.71 (s, 1H), 8.50- |
| | | | | 8.46 (m, 1H), 8.40 (t, J = 7.6 Hz, 1H), 8.35 (s, 1H), |
| | | | | 8.21 (d, J = 7.2 Hz, 1H), 7.42 (s, 1H), 7.09-6.86 |
| | | | | (m, 3H), 5.41-5.31 (m, 1H), 4.51-4.34 (m, 1H), |
| | | | | 3.64 (s, 3H), 3.23-3.14 (m, 2H), 3.07 (s, 4H), |
| | | | | 2.96-2.83 (m, 3H), 2.72-2.58 (m, 6H), 2.29- |
| | | | | 2.12 (m, 4H), 2.03-1.94 (m, 9H), 1.29-1.22 (m, |
| | | | | 2H), 1.18-1.11 (m, 1H) |
| I-393 | CET | BSC | 784.2 | 11.11 (s, 1H), 10.14 (s, 1H), 8.81-8.60 (m, 1H), |
| | | | | 8.46-8.37 (m, 3H), 8.24-8.20 (m, 2H), 7.52 (s, |
| | | | | 1H), 6.98-6.86 (m, 2H), 6.74 (d, J = 8.0 Hz, 1H), |
| | | | | 5.41-5.33 (m, 1H), 4.56-4.44 (m, 1H), 4.03 (s, |
| | | | | 2H), 3.71 (s, 3H), 3.62-3.54 (m, 2H), 3.46-3.40 |
| | | | | (m, 2H), 3.15 (d, J = 4.4 Hz, 2H), 2.96-2.85 (m, |
| | | | | 1H), 2.76-2.70 (m, 1H), 2.63 (d, J = 18.4 Hz, |
| | | | | 2H), 2.42 (s, 3H), 2.24-2.18 (m, 2H), 2.13-2.07 |
| | | | | (m, 3H), 2.04-1.94 (m, 6H), 1.42-1.27 (m, 2H) |
| I-395 | CCS | BVU | 788.4 | 11.08 (s, 1H), 9.10-9.00 (m, 1H), 8.88 (s, 1H), |
| | | | | 8.75 (s, 1H), 8.67 (s, 1H), 8.55-8.34 (m, 1H), |
| | | | | 8.01 (s, 1H), 7.93-7.82 (m, 1H), 7.19-7.07 (m, |
| | | | | 1H), 6.99-6.89 (m, 1H), 6.74 (d, J = 8.8 Hz, 1H), |
| | | | | 5.36-5.29 (m, 1H), 3.83 (s, 3H), 3.78-3.69 (m, |
| | | | | 3H), 3.61 (s, 3H), 3.53 (d, J = 11.6 Hz, 2H), 3.21- |
| | | | | 3.17 (m, 1H), 3.12-3.04 (m, 4H), 2.95-2.83 (m, |
| | | | | 1H), 2.75-2.67 (m, 1H), 2.65-2.57 (m, 1H), 2.01- |
| | | | | 1.85 (m, 6H), 1.84-1.76 (m, 1H), 1.45-1.36 (m, 2H), |
| | | | | 1.29 (d, J = 6.4 Hz, 6H), 1.21-1.08 (m, 3H) |
| I-396 | BWC | BSC | 770.3 | 11.10 (s, 1H), 10.13 (s, 1H), 8.45-8.36 (m, 3H), |
| | | | | 8.21 (dd, J = 1.6, 5.6 Hz, 2H), 7.53-7.47 (m, 2H), |
| | | | | 7.17-7.10 (m, 1H), 7.07-7.00 (m, 1H), 5.39 (dd, |
| | | | | J = 5.6, 12.0 Hz, 1H), 4.59 (s, 1H), 4.54-4.45 (m, |
| | | | | 1H), 4.44-4.39 (m, 1H), 3.88-3.74 (m, 2H), 3.63- |
| | | | | 3.54 (m, 5H), 3.43 (d, J = 6.0 Hz, 2H), 3.08 (t, J = |
| | | | | 5.6 Hz, 1H), 2.95-2.86 (m, 1H), 2.78-2.63 (m, |
| | | | | 3H), 2.40 (s, 3H), 2.23 (d, J = 8.4 Hz, 2H), 2.05- |
| | | | | 1.90 (m, 6H), 1.46-1.29 (m, 2H) |
| I-397 | BAI | CFV | 786.3 | 11.08 (s, 1H), 10.23 (s, 1H), 8.42 (d, 1H), 8.36 (d, |
| | | | | 1H), 8.37 (s, 1H), 8.20 (d, 1H), 8.14 (s, 1H), 7.54 |
| | | | | (s, 1H), 7.00-6.93 (q, 2H), 6.90 (d, 1H), 5.38- |
| | | | | 5.32 (m, 1H), 4.48-4.42 (m, 1H), 3.64 (s, 3H), |
| | | | | 3.21-3.15 (m, 2H), 3.01-2.84 (m, 6H), 2.76- |
| | | | | 2.68 (m, 2H), 2.66-2.62 (m, 1H), 2.61-2.58 (m, |
| | | | | 1H), 2.29-2.22 (m, 3H), 2.21-2.12 (m, 3H), 2.03- |
| | | | | 1.93 (m, 6H), 1.30 (s, 3H), 1.29 (s, 3H) |
| I-398 | BWS | BSC | 784.4 | 11.10 (s, 1H), 10.14 (s, 1H), 8.43 (d, 1H), 8.41 (t, |
| | | | | 2H), 8.20 (d, 2H), 7.52 (s, 1H), 7.14 (d, 1H), 7.03 |
| | | | | (t, J = 7.6 Hz, 1H), 6.95 (d, J = 7.6 Hz, 1H), 5.36 |
| | | | | (d, J = 12.4 Hz, 1H), 4.50-4.42 (m, 1H), 3.71 (s, |
| | | | | 3H), 2.96-2.83 (m, 3H), 2.75-2.70 (m, 1H), |
| | | | | 2.66-2.64 (m, 2H), 2.62-2.58 (m, 2H), 2.55-2.53 |
| | | | | (m, 3H), 2.41 (s, 3H), 2.20 (d, J = 10.4 Hz, 3H), |
| | | | | 2.11-1.91 (m, 9H), 1.28-1.18 (m, 2H) |
| I-401 | BAI | CFW | 759.2 | 11.09 (s, 1H), 10.20 (s, 1H), 9.58 (s, 1H), 9.48 (s, |
| | | | | 1H), 8.38 (s, 1H), 7.97 (s, 1H), 7.51 (s, 1H), 7.11- |
| | | | | 6.83 (m, 3H), 5.43-5.27 (m, 1H), 4.53-4.37 |
| | | | | (m, 1H), 3.64 (s, 3H), 3.22-3.10 (m, 2H), 3.04- |
| | | | | 2.82 (m, 5H), 2.64-2.58 (m, 2H), 2.37 (s, 3H), |
| | | | | 2.25-2.14 (m, 4H), 2.00-1.94 (m, 6H), 1.76- |
| | | | | 1.62 (m, 1H), 1.29-1.12 (m, 3H) |
| I-402 | CAK | BTW | 787.2 | 10.37 (s, 1H), 9.04-8.85 (m, 1H), 8.44-8.29 (m, |
| | | | | 4H), 8.19-8.17 (m, 1H), 7.66-7.52 (m, 2H), 7.01- |
| | | | | 6.95 (m, 1H), 6.75 (d, J = 8.8 Hz, 1H), 5.46- |
| | | | | 5.37 (m, 1H), 4.58-4.46 (m, 1H), 3.82 (s, 3H), |
| | | | | 3.67-3.53 (m, 6H), 3.18-3.13 (m, 1H), 3.07-2.93 |
| | | | | (m, 6H), 2.83-2.70 (m, 3H), 2.26-2.20 (m, 2H), 2.12- |
| | | | | 1.95 (m, 7H), 1.92-1.83 (m, 2H), 1.40-1.27 (m, 2H) |
| I-403 | BCD | BSC | 771.3 | 10.13 (s, 1H), 9.00-8.89 (m, 1H), 8.47-8.33 (m, |
| | | | | 3H), 8.25-8.17 (m, 2H), 7.52 (s, 1H), 7.12-6.92 |
| | | | | (m, 3H), 5.46 (dd, J = 5.6, 12.8 Hz, 1H), 4.56- |
| | | | | 4.41 (m, 1H), 3.67-3.58 (m, 5H), 3.24-3.17 (m, |
| | | | | 2H), 3.10-3.02 (m, 5H), 2.99-2.92 (m, 1H), 2.84- |
| | | | | 2.70 (m, 2H), 2.41 (s, 3H), 2.24-2.17 (m, 2H), |
| | | | | 2.12-1.96 (m, 10H), 1.35-1.22 (m, 2H) |
| I-404 | CFX | BSC | 772.3 | 10.13 (s, 1H), 8.45-8.36 (m, 3H), 8.20 (d, 2H), |
| | | | | 7.51 (s, 1H), 7.03-6.88 (m, 3H), 5.43 (d, J = 12.8 |
| | | | | Hz, 1H), 4.50-4.40 (m, 1H), 3.65 (s, 3H), 3.03 (s, |
| | | | | 3H), 3.02-2.90 (m, 4H), 2.82-2.78 (m, 1H), 2.74- |
| | | | | 2.64 (m, 2H), 2.62-2.58 (m, 3H), 2.41 (s, 3H), 2.32- |
| | | | | 2.11 (m, 5H), 2.06-1.86 (m, 6H), 1.27-1.10 (m, 2H) |
| I-405 | BAI | CFZ | 802.2 | 12.54 (s, 1H), 11.10 (s, 1H), 9.08 (s, 1H), 8.59 (d, |
| | | | | J = 0.8 Hz, 1H), 8.50-8.44 (m, 2H), 8.40 (t, J = |
| | | | | 8.0 Hz, 1H), 8.21 (dd, J = 0.8, 7.6 Hz, 1H), 7.08- |
| | | | | 6.91 (m, 3H), 5.37 (dd, J = 4.8, 12.4 Hz, 1H), 4.66- |
| | | | | 4.51 (m, 1H), 3.96 (s, 3H), 3.65 (s, 3H), 3.30-3.27 |
| | | | | (m, 2H), 3.24-3.08 (m, 4H), 2.97-2.83 (m, 2H), 2.78- |
| | | | | 2.68 (m, 1H), 2.66-2.57 (m, 1H), 2.52 (s, 1H), 2.27- |
| | | | | 2.20 (m, 2H), 2.09-1.93 (m, 7H), 1.43-1.19 (m, 3H) |
| I-406 | BAI | CGA | 770.3 | 11.10 (s, 1H), 8.81 (s, 1H), 8.67-8.54 (m, 2H), |
| | | | | 8.34 (t, J = 8.0 Hz, 1H), 8.22 (s, 1H), 8.15 (d, J = |
| | | | | 7.6 Hz, 1H), 7.06-6.79 (m, 3H), 5.36 (dd, J = 5.2, |
| | | | | 12.8 Hz, 1H), 4.66 (t, J = 11.2 Hz, 1H), 3.64 (s, 3H), |
| | | | | 3.10-2.78 (m, 7H), 2.77-2.58 (m, 3H), 2.30-2.19 (m, 5H), |
| | | | | 2.08-1.97 (m, 5H), 1.79-1.63 (m, 1H), 1.25-1.11 (m, 2H) |
| I-411 | BAI | CGD | 782.3 | 11.10 (s, 1H), 9.43-9.16 (m, 1H), 9.02-8.83 (m, |
| | | | | 2H), 8.77 (s, 1H), 8.70 (s, 1H), 8.05 (s, 1H), 7.96- |
| | | | | 7.86 (m, 1H), 7.17 (d, J = 4.8 Hz, 1H), 7.09- |
| | | | | 7.01 (m, 1H), 6.99-6.90 (m, 2H), 5.38 (dd, J = |
| | | | | 5.2, 12.4 Hz, 1H), 4.74-4.53 (m, 1H), 4.14-3.91 |
| | | | | (m, 1H), 3.65 (s, 5H), 3.27-3.07 (m, 6H), 2.97- |
| | | | | 2.84 (m, 1H), 2.77-2.68 (m, 1H), 2.61 (s, 1H), |
| | | | | 2.31-2.25 (m, 2H), 2.12-1.83 (m, 7H), 1.37 (d, |
| | | | | J = 6.4 Hz, 6H), 1.36-1.29 (m, 3H) |
| I-412 | BAI | CFY | 786.4 | 13.15 (s, 1H), 11.10 (s, 1H), 9.08 (s, 1H), 8.67 (s, |
| | | | | 1H), 8.56 (s, 1H), 8.47-8.43 (m, 1H), 8.40-8.35 |
| | | | | (m, 1H), 8.20 (d, J = 7.6 Hz, 1H), 7.04-6.84 (m, |
| | | | | 3H), 5.36 (dd, J = 5.6, 12.4 Hz, 1H), 4.64-4.46 |
| | | | | (m, 1H), 3.64 (s, 3H), 3.16 (s, 1H), 3.04-2.84 (m, |
| | | | | 6H), 2.80 (s, 3H), 2.65 (d, J = 11.6 Hz, 2H), 2.30- |
| | | | | 2.13 (m, 6H), 2.04-1.93 (m, 5H), 1.77-1.59 (m, 1H), |
| | | | | 1.24-1.12 (m, 2H) |
| I-413 | CGF | ATJ | 774.3 | 10.52 (d, J = 7.6 Hz, 2H), 9.42 (s, 1H), 8.71 (s, |
| | | | | 1H), 8.60 (s, 1H), 8.49-8.37 (m, 2H), 8.35 (s, |
| | | | | 1H), 8.22 (d, J = 7.6 Hz, 1H), 7.51 (d, J = 9.6 Hz, |
| | | | | 1H), 7.29 (d, J = 9.2 Hz, 1H), 7.15 (s, 1H), 4.51- |
| | | | | 4.38 (m, 1H), 3.99 (s, 3H), 3.97-3.87 (m, 1H), |
| | | | | 3.80-3.45 (m, 6H), 3.40-3.33 (m, 2H), 3.21 (s, |
| | | | | 3H), 3.03 (m, 1H), 2.77-2.68 (m, 1H), 2.21 (d, J = |
| | | | | 11.2 Hz, 2H), 2.10-1.89 (m, 5H), 1.39-1.22 (m, 2H) |
| I-414 | CGG | BSC | 770.4 | 11.07 (s, 1H), 10.13 (s, 1H), 8.45-8.34 (m, 3H), |
| | | | | 8.26-8.18 (m, 2H), 7.51 (s, 1H), 6.97-6.90 (m, |
| | | | | 1H), 6.79 (d, J = 8.0 Hz, 1H), 6.69 (d, J = 7.6 Hz, |
| | | | | 1H), 5.31 (dd, J = 5.4, 12.4 Hz, 1H), 4.46-4.33 |
| | | | | (m, 1H), 4.15 (d, J = 5.6 Hz, 2H), 3.51 (s, 3H), |
| | | | | 3.08 (d, J = 9.6 Hz, 2H), 2.97 (dd, J = 3.6, 10.0 |
| | | | | Hz, 2H), 2.93-2.83 (m, 1H), 2.82-2.74 (m, 1H), |
| | | | | 2.64-2.56 (m, 2H), 2.40 (s, 3H), 2.38 (d, J = 7.2 |
| | | | | Hz, 2H), 2.15-2.06 (m, 3H), 2.02-1.94 (m, 1H), 1.93- |
| | | | | 1.81 (m, 4H), 1.69-1.59 (m, 1H), 1.13-1.00 (m, 2H) |
| I-419 | AQK | BXS | 826.4 | 11.09 (s, 1H), 10.06 (s, 1H), 9.36 (d, J = 1.6 Hz, |
| | | | | 1H), 9.21 (d, J = 1.6 Hz, 1H), 8.80 (s, 1H), 8.25 |
| | | | | (s, 1H), 7.65 (s, 1H), 7.00-6.86 (m, 4H), 5.35 (dd, |
| | | | | J = 5.2, 12.8 Hz, 1H), 4.47 (s, 1H), 4.42-4.33 (m, |
| | | | | 1H), 4.31 (s, 1H), 4.01 (d, J = 7.2 Hz, 1H), 3.72 |
| | | | | (d, J = 6.4 Hz, 1H), 3.64 (s, 3H), 3.43 (d, J = 8.0 |
| | | | | Hz, 2H), 3.19-3.15 (m, 2H), 2.99 (d, J = 9.6 Hz, |
| | | | | 1H), 2.95-2.83 (m, 2H), 2.77-2.70 (m, 3H), 2.63 |
| | | | | (d, J = 3.6 Hz, 1H), 2.17-2.12 (m, 2H), 2.07 (s, |
| | | | | 3H), 2.00-1.95 (m, 3H), 1.92 (d, J = 3.6 Hz, 1H), |
| | | | | 1.89 (d, J = 8.0 Hz, 2H), 1.86-1.81 (m, 2H), 1.77 |
| | | | | (d, J = 10.4 Hz, 2H), 1.71-1.66 (m, 2H), 1.63- |
| | | | | 1.58 (m, 1H), 1.18-1.09 (m, 2H) |
| I-420 | BAI | CGI | 780.4 | 11.09 (s, 1H), 9.14 (s, 1H), 8.86-8.78 (m, 2H), |
| | | | | 8.52 (s, 1H), 7.85 (s, 1H), 7.06-6.87 (m, 3H), |
| | | | | 5.40-5.31 (m, 1H), 4.63-4.52 (m, 1H), 4.46-4.35 |
| | | | | (m, 1H), 3.64 (s, 3H), 3.12-2.83 (m, 6H), 2.77- |
| | | | | 2.60 (m, 3H), 2.52 (s, 2H), 2.30-2.22 (m, 3H), 2.10- |
| | | | | 1.96 (m, 4H), 1.95-1.86 (m, 2H), 1.86-1.61 (m, |
| | | | | 2H), 1.32 (d, J = 6.4 Hz, 6H), 1.28-1.14 (m, 2H) |
| I-422^{h} | BAI | BJB | 822.3 | 11.54 (s, 1H), 8.93 (s, 1H), 8.59 (s, 1H), 8.50 (d, J = |
| | | | | 8.0 Hz, 1H), 8.16 (t, J = 8.0 Hz, 1H), 8.09-8.05 |
| | | | | (m, 2H), 7.92 (d, J = 8.0 Hz, 1H), 7.08-6.99 (m, |
| | | | | 2H), 6.64 (m, 1H), 5.21 (dd, J = 5.6, 12.0 Hz, 1H), |
| | | | | 4.54.4.43 (ₘ, 1H), 3.77 (s, 3H), 3.29-3.18 (m, |
| | | | | 2H), 3.17 (s, 3H), 3.13 (m, 2H), 3.00-2.91 (m, |
| | | | | 2H), 2.90-2.83 (m, 1H), 2.81-2.72 (m, 2H), 2.67- |
| | | | | 2.49 (m, 2H), 2.43-2.33 (m, 2H), 2.28-2.15 |
| | | | | (m, 3H), 2.11-2.00 (m, 2H), 1.94-1.84 (m, 1H), |
| | | | | 1.38 (m, 2H), 1.34-1.28 (m, 1H), 1.26-1.21 (m, 1H) |
| I-423 | BWT | BRP | 756.3 | 11.09 (s, 1H), 10.36 (s, 1H), 8.44-8.33 (m, 3H), |
| | | | | 8.30 (d, J = 0.8 Hz, 1H), 8.18 (dd, J = 1.2, 7.6 Hz, |
| | | | | 1H), 7.65-7.58 (m, 1H), 7.58-7.51 (m, 1H), 6.99 |
| | | | | (d, J = 4.4 Hz, 2H), 6.85 (d, J = 4.0 Hz, 1H), 5.39- |
| | | | | 5.30 (m, 1H), 4.52-4.41 (m, 1H), 3.89 (s, 2H), |
| | | | | 3.63 (d, J = 1.2 Hz, 3H), 3.59 (d, J = 10.8 Hz, 1H), |
| | | | | 3.15 (d, J = 8.8 Hz, 1H), 3.01-2.81 (m, 3H), 2.79- |
| | | | | 2.57 (m, 4H), 2.19 (d, J = 12.0 Hz, 2H), 2.09- |
| | | | | 1.88 (m, 7H), 1.74-1.58 (m, 1H), 1.29-1.13 (m, 2H) |
| I-425 | AQK | CGY | 791.3 | 11.16-10.99 (m, 1H), 10.51 (s, 1H), 9.01 (s, 1H), |
| | | | | 8.52-8.41 (m, 2H), 8.25 (d, J = 0.8 Hz, 1H), 7.74 |
| | | | | (s, 1H), 7.04-6.82 (m, 3H), 5.39-5.29 (m, 1H), |
| | | | | 4.03 (s, 3H), 3.84 (s, 1H), 3.63 (s, 3H), 2.72-2.62 |
| | | | | (m, 7H), 2.37-2.30 (m, 4H), 2.24 (s, 3H), 2.06- |
| | | | | 1.97 (m, 3H), 1.86-1.75 (m, 3H), 1.71-1.57 (m, 3H) |
| I-426 | CEQ | ATJ | 800.4 | 11.07 (s, 1H), 10.50 (s, 1H), 8.69 (s, 1H), 8.49- |
| | | | | 8.44 (m, 1H), 8.43-8.37 (m, 1H), 8.34 (s, 1H), |
| | | | | 8.22 (d, J = 7.6 Hz, 1H), 7.16 (s, 1H), 7.03-6.96 |
| | | | | (m, 1H), 6.89 (d, J = 7.6 Hz, 1H), 6.82 (d, J = 7.6 |
| | | | | Hz, 1H), 5.42-5.23 (m, 1H), 5.12-4.71 (m, 1H), |
| | | | | 4.44-4.31 (m, 1H), 3.98 (s, 3H), 3.25-3.14 (m, |
| | | | | 2H), 3.06-2.80 (m, 6H), 2.65-2.56 (m, 2H), 2.27- |
| | | | | 2.10 (m, 5H), 2.03-1.87 (m, 5H), 1.73-1.59 |
| | | | | (m, 1H), 1.21-0.89 (m, 6H) |
| I-427 | BAI | CCR | 792.0 | 11.09 (s, 1H), 10.34 (s, 1H), 8.72 (s, 1H), 8.53- |
| | | | | 8.43 (m, 1H), 8.43-8.32 (m, 1H), 8.22 (d, J = 7.6 |
| | | | | Hz, 1H), 7.28 (s, 1H), 7.10-6.98 (m, 1H), 6.98- |
| | | | | 6.94 (m, 1H), 6.94-6.80 (m, 1H), 5.44-5.25 (m, 1H), |
| | | | | 4.10-3.99 (m, 2H), 3.96 (s, 3H), 3.63 (s, 3H), 3.23- |
| | | | | 3.09 (m, 4H), 3.02-2.81 (m, 5H), 2.64 (s, 3H), 2.26 |
| | | | | (d, J = 3.6 Hz, 2H), 2.04-1.95 (m, 1H), 1.95-1.80 |
| | | | | (m, 3H), 1.44-1.30 (m, 1H), 1.28-1.15 (m, 2H) |
| I-428 | CJA | ATJ | 759.2 | 11.10 (s, 1H), 10.50 (s, 1H), 8.69 (s, 1H), 8.48- |
| | | | | 8.37 (m, 2H), 8.34 (s, 1H), 8.22 (d, J = 7.8 Hz, |
| | | | | 1H), 7.21 (s, 1H), 7.14 (s, 1H), 7.11-7.00 (m, |
| | | | | 2H), 5.37 (dd, J = 5.2, 12.8 Hz, 1H), 4.42 (t, J = |
| | | | | 11.6 Hz, 1H), 3.98 (s, 3H), 3.79-3.41 (m, 3H), |
| | | | | 3.36 (s, 3H), 3.21-3.01 (m, 2H), 2.99-2.79 (m, |
| | | | | 2H), 2.78-2.59 (m, 3H), 2.44-2.35 (m, 1H), 2.18 |
| | | | | (d, J = 11.6 Hz, 2H), 2.08-1.88 (m, 6H), 1.83 (s, |
| | | | | 1H), 1.39-1.11 (m, 2H) |

^{a}For Method 2, when the amine is the HCl salt, TEA was added to free base the salt, followed by HOAc to adjust the pH to 3-4. KOAc could also be used in place of the TEA/HOAc combination. Step 1 was run anywhere from 0.5-48 hrs and the reaction temperature was run anywhere from −15° C. to rt. The final products were isolated under standard purification techniques including reverse HPLC, silica gel chromatography, and prep-TLC with appropriate solvent conditions. ^{b}No DMF used as a co-solvent. ^{c}No DMF used as a co-solvent. ^{d}For the reductive amination tetraisopropoxytitanium was used and the mixture was stirred at 80° C. for 16 hrs. Then the reaction mixture was cooled to 25° C., and NaBH₃CN was added and the mixture was stirred at 25° C. for 8 hrs. ^{e}LCMS reported as (M—H₂O)⁺ ion. ^{f}Reaction run for 96 hr at rt. ^{g}DIEA used as the base. ^{h}CDCl₃ used as the solvent for the NMR data.

### Example 3 (Method 5): Synthesis of 5-cyano-N-[2-[4-[[[1-[1-(2,6-dioxo-3-piperidyl)-3-methyl-2-oxo-benzimidazol-4-yl]-4-piperidyl]-methyl-amino]methyl]cyclohexyl]-6-methoxy-indazol-5-yl]pyridine-3-carboxamide (I-71)

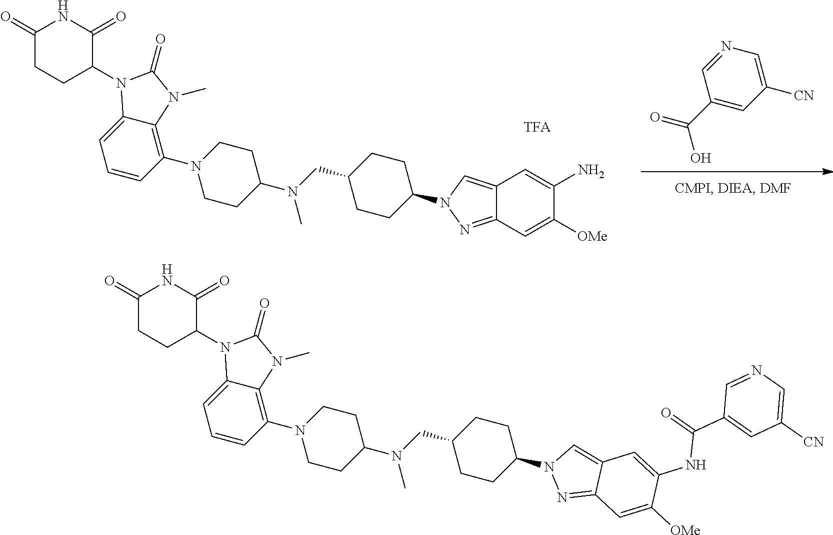 [SMILES: COc1cc2nn([C@H]3CC[C@H](CN(C)C4CCN(c5cccc6c5n(C)c(=O)n6C5CCC(=O)NC5=O)CC4)CC3)cc2cc1N ; N#Cc1cncc(C(=O)O)c1 ; COc1cc2nn([C@H]3CC[C@H](CN(C)C4CCN(c5cccc6c5n(C)c(=O)n6C5CCC(=O)NC5=O)CC4)CC3)cc2cc1NC(=O)c1cncc(C#N)c1]

To a solution of 5-cyanopyridine-3-carboxylic acid (11.9 mg, 80.7 umol), CMPI (24.7 mg, 96.9 umol) and DIEA (52.2 mg, 403 umol) in DMF (1 mL) was added 3-[4-[4-[[4-(5-amino-6-methoxy-indazol-2-yl)cyclohexyl]methyl-methyl-amino]-1-piperidyl]-3-methyl-2-oxo-benzimidazol-1-yl]piperidine-2,6-dione (60.0 mg, 80.7 umol, TFA, Intermediate BGU). The reaction mixture was then stirred at 25° C. for 1.5 hrs. On completion, the reaction mixture was concentrated in vacuo to give a residue. The residue was purified by prep-HPLC (column: Phenomenex luna C18 150*25 mm*10 um; mobile phase: [water (0.225% FA)-ACN]; B %: 10%-40%, 11.5 min) to give the title compound (27.5 mg, 42% yield) as an off-white solid. ¹HNMR (400 MHz, DMSO-d₆) δ 11.09 (s, 1H), 9.90 (s, 1H), 9.31 (s, 1H), 9.21 (d, J=1.6 Hz, 1H), 8.88-8.73 (m, 1H), 8.32 (s, 1H), 8.08 (s, 1H), 7.08 (s, 1H), 7.02-6.95 (m, 1H), 6.95-6.85 (m, 2H), 5.35 (dd, J=5.2, 12.4 Hz, 1H), 4.46-4.32 (m, 1H), 3.88 (s, 3H), 3.65 (s, 3H), 3.27 (s, 3H), 3.18 (d, J=10.0 Hz, 2H), 2.94-2.84 (m, 1H), 2.80-2.69 (m, 3H), 2.66-2.56 (m, 2H), 2.43-2.34 (m, 2H), 2.16 (d, J=11.2 Hz, 2H), 2.03-1.83 (m, 7H), 1.80-1.57 (m, 3H), 1.24-1.07 (m, 2H); LC-MS (ESI⁺) m/z 759.2 (M+H)⁺.

The following compounds in Table 5 where prepared according to the same procedure of Method 5 above.

TABLE 5
Compounds prepared by Method 5.

| | Inter- | Inter- | LCMS | |
|---|---|---|---|---|
| | mediate | mediate | (ES+) m/z | |
| I-# | Amine | Acid | (M + H)⁺ | 1H NMR (400 MHz, DMSO-d6) δ |
| I-34 | BGU | BQJ | 803.3 | 11.10 (s, 1H), 10.29 (s, 1H), 9.89 (d, J = 1.6 Hz, |
| | | | | 1H), 8.65 (d, J = 1.6 Hz, 1H), 8.34 (s, 1H), 8.11 |
| | | | | (s, 1H), 7.11 (s, 1H), 7.01-6.86 (m, 3H), 5.35 |
| | | | | (dd, J = 5.2, 12.4 Hz, 1H), 4.45-4.33 (m, 1H), |
| | | | | 3.88 (s, 3H), 3.65 (s, 3H), 3.16 (d, J = 11.6 Hz, |
| | | | | 2H), 2.93-2.85 (m, 1H), 2.77-2.71 (m, 2H), |
| | | | | 2.68-2.58 (m, 2H), 2.46-2.42 (m, 1H), 2.31- |
| | | | | 2.29 (m, 2H), 2.26 (s, 3H), 2.18-2.14 (m, 2H), |
| | | | | 2.05-1.92 (m, 4H), 1.90-1.88 (m, 1H), 1.85- |
| | | | | 1.77 (m, 2H), 1.73-1.62 (m, 2H), 1.60-1.53 |
| | | | | (m, 1H), 1.17-1.06 (m, 2H) |
| I-77 | BSS | 5-cyanopyridine- | 730.5 | 11.1 (s, 1H), 9.90 (s, 1H), 9.31 (s, 1H), 9.21 (d, |
| | | 3-carboxylic acid | | J = 1.6 Hz, 1H), 8.78 (s, 1H), 8.31 (s, 1H), 8.08 |
| | | (CAS# 887579-62-6) | | (s, 1H), 7.12-7.06 (m, 1H), 7.06-6.94 (m, 3H), |
| | | | | 5.37 (dd, J = 5.2, 12.8 Hz, 1H), 4.45-4.31 (m, |
| | | | | 1H), 3.88 (s, 3H), 3.59 (s, 3H), 3.05-2.83 (m, 4H), |
| | | | | 2.75 (d, J = 3.2 Hz, 1H), 2.65-2.59 (m, 1H), 2.23 |
| | | | | (d, J = 6.8 Hz, 2H), 2.18-2.06 (m, 4H), 2.01-1.86 |
| | | | | (m, 5H), 1.84-1.62 (m, 5H), 1.20-1.11 (m, 2H) |
| I-80 | BGU | 5-chloropyridine- | 768.4 | 11.09 (s, 1H), 9.83 (s, 1H), 9.05 (s, 1H), 8.83 (d, |
| | | 3-carboxylic acid | | J = 2.4 Hz, 1H), 8.41 (s, 1H), 8.31 (s, 1H), 8.03 |
| | | (CAS# 22620-27-5) | | (s, 1H), 7.07 (s, 1H), 7.01-6.94 (m, 1H), 6.94- |
| | | | | 6.84 (m, 2H), 5.35 (dd, J = 5.6, 12.8 Hz, 1H), |
| | | | | 4.45-4.32 (m, 1H), 3.87 (s, 3H), 3.65 (s, 3H), |
| | | | | 3.31-3.28 (m, 3H), 3.17 (d, J = 11.2 Hz, 2H), |
| | | | | 2.96-2.83 (m, 1H), 2.80-2.67 (m, 3H), 2.66- |
| | | | | 2.53 (m, 2H), 2.41-2.34 (m, 2H), 2.16 (d, J = |
| | | | | 10.8 Hz, 2H), 2.03-1.80 (m, 7H), 1.77-1.57 |
| | | | | (m, 3H), 1.21-1.06 (m, 2H) |
| I-81 | BGU | 5-fluoropyridine- | 752.4 | 11.10 (s, 1H), 9.81 (s, 1H), 9.00 (s, 1H), 8.96- |
| | | 3-carboxylic acid | | 8.83 (m, 1H), 8.80 (d, J = 2.8 Hz, 1H), 8.32 (s, |
| | | (CAS# 402-66-4) | | 1H), 8.21 (d, J = 9.6 Hz, 1H), 7.06 (s, 1H), 7.04- |
| | | | | 6.97 (m, 1H), 6.96-6.89 (m, 2H), 5.36 (dd, J = |
| | | | | 5.6, 12.8 Hz, 1H), 4.49-4.36 (m, 1H), 3.88 |
| | | | | (s, 3H), 3.66 (s, 3H), 3.28-3.19 (m, 4H), 3.05- |
| | | | | 2.95 (m, 1H), 2.94-2.77 (m, 6H), 2.76-2.68 (m, |
| | | | | 1H), 2.65-2.57 (m, 1H), 2.23-2.16 (m, 2H), 2.15- |
| | | | | 2.03 (m, 3H), 2.03-1.86 (m, 7H), 1.41-1.19 (m, 2H) |
| I-86 | BSS | 5-(trifluoromethyl)pyridine- | 773.6 | 11.1 (s, 1H), 10.0 (s, 1H), 9.37 (s, 1H), 9.20- |
| | | 3-carboxylic acid | | 9.13 (m, 1H), 8.67 (s, 1H), 8.32 (s, 1H), 8.03 (s, |
| | | (CAS# 131747-40-5) | | 1H), 7.08 (s, 1H), 7.06-6.97 (m, 3H), 5.38 (dd, |
| | | | | J = 5.6, 12.4 Hz, 1H), 4.46-4.35 (m, 1H), 3.87 |
| | | | | (s, 3H), 3.61 (s, 3H), 3.31-3.29 (m, 4H), 2.93-2.85 |
| | | | | (m, 1H), 2.74-2.68 (m, 1H), 2.67-2.58 (m, 2H), 2.17 |
| | | | | (d, J = 11.6 Hz, 3H), 2.04-1.94 (m, 5H), 1.93- |
| | | | | 1.81 (m, 5H), 1.80-1.69 (m, 1H), 1.26-1.14 (m, 2H) |
| I-92 | BGU | 5-(trifluoromethyl)pyridine- | 802.5 | 11.10 (s, 1H), 10.03 (s, 1H), 9.38 (s, 1H), 9.19 |
| | | 3-carboxylic acid | | (s, 1H), 8.68 (s, 1H), 8.34 (s, 1H), 8.04 (s, 1H), |
| | | (CAS# 131747-40-5) | | 7.08 (s, 1H), 7.04-6.97 (m, 1H), 6.96-6.87 (m, 2H), |
| | | | | 5.37 (dd, J = 5.6, 12.8 Hz, 1H), 4.48-4.36 (m, |
| | | | | 1H), 3.88 (s, 3H), 3.66 (s, 3H), 3.30 (s, 3H), 3.23 |
| | | | | (d, J = 11.2 Hz, 2H), 2.97-2.71 (m, 6H), 2.67- |
| | | | | 2.59 (m, 2H), 2.19 (d, J = 11.2 Hz, 2H), 2.09- |
| | | | | 1.90 (m, 7H), 1.89-1.71 (m, 3H), 1.33-1.14 (m, 2H) |
| I-93 | BSS | 5-chloropyridine- | 739.1 | 11.10 (s, 1H), 9.84 (s, 1H), 9.04 (s, 1H), 8.83 (d, |
| | | 3-carboxylic acid | | J = 2.4 Hz, 1H), 8.41 (s, 1H), 8.31 (s, 1H), 8.04 |
| | | (CAS# 22620-27-5) | | (s, 1H), 7.07 (s, 1H), 7.06-6.97 (m, 3H), 5.38 |
| | | | | (dd, J = 5.2, 12.4 Hz, 1H), 4.48-4.35 (m, 1H), |
| | | | | 3.87 (s, 3H), 3.61 (s, 3H), 2.97-2.67 (m, 4H), |
| | | | | 2.65-2.57 (m, 2H), 2.52 (s, 2H), 2.18 (d, J = |
| | | | | 10.8 Hz, 2H), 2.05-1.86 (m, 10H), 1.82 (s, 1H), |
| | | | | 1.29-1.15 (m, 3H) |
| I-130 | BGU | CBV | 802.5 | 11.10 (s, 1H), 10.03 (s, 1H), 9.38 (s, 1H), 9.19 |
| | | | | (s, 1H), 8.68 (s, 1H), 8.34 (s, 1H), 8.04 (s, 1H), |
| | | | | 7.08 (s, 1H), 7.04-6.97 (m, 1H), 6.96-6.87 (m, |
| | | | | 2H), 5.37 (dd, J = 5.6, 12.8 Hz, 1H), 4.48-4.36 |
| | | | | (m, 1H), 3.88 (s, 3H), 3.66 (s, 3H), 3.30 (s, 3H), |
| | | | | 3.23 (d, J = 11.2 Hz, 2H), 2.97-2.71 (m, 6H), |
| | | | | 2.67-2.59 (m, 2H), 2.19 (m, 2H), 2.09-1.90 |
| | | | | (m, 7H), 1.89-1.71 (m, 3H), 1.33-1.14 (m, 2H) |
| I-131 | BGU | BTX | 819.6 | 11.10 (s, 1H), 9.89 (s, 1H), 8.32 (s, 1H), 8.18 (s, |
| | | | | 1H), 8.12 (d, J = 8.8 Hz, 1H), 8.02-7.92 (m, |
| | | | | 2H), 7.08 (s, 1H), 7.03-6.96 (m, 1H), 6.96- |
| | | | | 6.85 (m, 2H), 5.36 (dd, J = 5.6, 12.8 Hz, 1H), |
| | | | | 4.47-4.34 (m, 1H), 3.87 (s, 3H), 3.65 (s, 3H), |
| | | | | 3.29 (s, 3H), 3.20 (d, J = 10.0 Hz, 2H), 2.96- |
| | | | | 2.69 (m, 5H), 2.69-2.57 (m, 3H), 2.17 (d, J = |
| | | | | 10.4 Hz, 2H), 2.06-1.87 (m, 7H), 1.86-1.61 |
| | | | | (m, 3H), 1.30-1.08 (m, 2H) |
| I-132 | BSS | BTX | 790.6 | 11.09 (s, 1H), 9.88 (s, 1H), 8.31 (s, 1H), 8.18 (s, |
| | | | | 1H), 8.11 (d, J = 8.8 Hz, 1H), 8.02-7.91 (m, |
| | | | | 2H), 7.08 (s, 1H), 7.05-6.94 (m, 3H), 5.37 (m, |
| | | | | 1H), 4.46-4.33 (m, 1H), 3.86 (s, 3H), 3.59 (s, |
| | | | | 3H), 3.23 (s, 1H), 3.00 (d, J = 10.8 Hz, 2H), 2.95- |
| | | | | 2.83 (m, 1H), 2.77-2.62 (m, 2H), 2.22 (d, J = |
| | | | | 7.2 Hz, 2H), 2.16 (d, J = 11.2 Hz, 2H), 2.12- |
| | | | | 2.04 (m, 2H), 2.02-1.95 (m, 3H), 1.95-1.86 |
| | | | | (m, 2H), 1.84-1.74 (m, 4H), 1.71-1.61 (m, |
| | | | | 1H), 1.21-1.06 (m, 2H) |
| I-133 | BUP | BUN | 700.5 | 11.09 (s, 1H), 10.51 (s, 1H), 9.33 (d, J = 2.0 Hz, |
| | | | | 1H), 9.21 (d, J = 2.0 Hz, 1H), 8.82 (t, J = 2.4 |
| | | | | Hz, 1H), 8.40 (s, 1H), 8.26 (d, J = 1.2 Hz, 1H), |
| | | | | 7.62 (d, J = 9.2 Hz, 1H), 7.44 (dd, J = 2.0, 9.2 |
| | | | | Hz, 1H), 7.07-6.91 (m, 3H), 5.37 (dd, J = 5.2, |
| | | | | 12.8 Hz, 1H), 4.50-4.39 (m, 1H), 3.52 (s, 3H), |
| | | | | 3.24-3.19 (m, 1H), 3.05-2.96 (m, 2H), 2.94- |
| | | | | 2.83 (m, 1H), 2.77-2.58 (m, 2H), 2.27-2.20 |
| | | | | (m, 2H), 2.10-2.14 (m, 2H), 2.13-2.06 (m, |
| | | | | 2H), 2.02-1.88 (m, 5H), 1.86-1.75 (m, 4H), |
| | | | | 1.72-1.61 (m, 1H), 1.21-1.07 (m, 2H) |
| I-134 | BSS | BUX | 753.5 | 11.09 (s, 1H), 9.75 (s, 1H), 8.94 (s, 1H), 8.36 (s, |
| | | | | 1H), 8.30 (s, 1H), 8.01 (s, 1H), 7.07 (s, 1H), 7.05- |
| | | | | 7.00 (m, 2H), 6.99 (d, J = 3.6 Hz, 1H), 5.37 |
| | | | | (dd, J = 5.6, 12.4 Hz, 1H), 4.44-4.33 (m, 1H), |
| | | | | 3.87 (s, 3H), 3.59 (s, 3H), 3.25 (d, J = 4.4 Hz, |
| | | | | 1H), 3.03 (d, J = 11.2 Hz, 2H), 2.94-2.83 (m, |
| | | | | 1H), 2.77-2.67 (m, 1H), 2.63 (s, 3H), 2.61- |
| | | | | 2.57 (m, 1H), 2.26 (d, J = 6.8 Hz, 2H), 2.15 (d, |
| | | | | J = 11.2 Hz, 4H), 1.98 (d, J = 12.4 Hz, 3H), |
| | | | | 1.94-1.86 (m, 2H), 1.85-1.76 (m, 4H), 1.68 (d, |
| | | | | J = 1.2 Hz, 1H), 1.21-1.07 (m, 2H) |
| I-135 | BSS | BUZ | 740.3 | 11.09 (s, 1H), 10.59 (s, 1H), 9.66 (d, J = 2.4 Hz, |
| | | | | 1H), 8.67 (s, 1H), 8.49 (d, J = 2.4 Hz, 1H), 8.35 |
| | | | | (s, 1H), 7.17 (s, 1H), 7.06-6.93 (m, 3H), 5.37 |
| | | | | (J = 5.6, 12.0 Hz, 1H), 4.44-4.35 (m, 1H), 4.01 |
| | | | | (s, 3H), 3.59 (s, 3H), 3.26-3.19 (m, 2H), 3.02 |
| | | | | (d, J = 12.0 Hz, 2H), 2.94-2.83 (m, 1H), 2.75- |
| | | | | 2.61 (m, 2H), 2.24 (d, J = 8.0 Hz, 2H), 2.19- |
| | | | | 2.08 (m, 4H), 1.98 (d, J = 12.0 Hz, 3H), 1.91 (d, |
| | | | | J = 12.0 Hz, 1H), 1.77 (s, 4H), 1.67 (d, J = 3.6 |
| | | | | Hz, 1H), 1.21-1.09 (m, 2H) |
| I-136 | BSS | BVF | 764.8 | 11.09 (s, 1H), 10.04 (s, 1H), 9.14 (d, J = 1.2 Hz, |
| | | | | 1H), 8.71 (d, J = 1.6 Hz, 1H), 8.32 (s, 1H), 8.06 |
| | | | | (s, 1H), 7.09 (s, 1H), 7.05-6.95 (m, 3H), 5.37 |
| | | | | (dd, J = 5.2, 12.6 Hz, 1H), 4.45-4.33 (m, 1H), |
| | | | | 3.87 (s, 3H), 3.59 (s, 3H), 3.25-3.19 (m, 1H), |
| | | | | 3.04-2.97 (m, 2H), 2.94-2.84 (m, 1H), 2.77- |
| | | | | 2.69 (m, 1H), 2.62 (d, J = 17.0 Hz, 1H), 2.23 (d, |
| | | | | J = 7.0 Hz, 2H), 2.19-2.06 (m, 4H), 2.02-1.95 |
| | | | | (m, 3H), 1.94-1.85 (m, 2H), 1.84-1.75 (m, 4H), |
| | | | | 1.67 (dd, J = 3.6, 10.4 Hz, 1H), 1.20-1.08 (m, 2H) |
| I-174 | BXA | BXC | 722.3 | 11.11 (s, 1H), 9.48 (d, J = 9.6 Hz, 1H), 8.51 (s, |
| | | | | 1H), 8.31 (s, 1H), 7.92 (t, J = 7.6 Hz, 1H), 7.67- |
| | | | | 7.60 (m, 1H), 7.44-7.36 (m, 2H), 7.11-7.06 |
| | | | | (m, 2H), 7.06-7.02 (m, 1H), 6.96 (d, J = 8.2 |
| | | | | Hz, 1H), 5.40 (dd, J = 5.4, 12.4 Hz, 1H), 4.48- |
| | | | | 4.38 (m, 1H), 3.93 (s, 3H), 3.68-3.55 (m, 6H), |
| | | | | 3.25-3.16 (m, 2H), 3.04 (d, J = 0.8 Hz, 2H), |
| | | | | 2.94-2.84 (m, 1H), 2.77-2.68 (m, 1H), 2.68- |
| | | | | 2.64 (m, 1H), 2.24-2.13 (m, 4H), 2.08-1.91 |
| | | | | (m, 8H), 1.30 (d, J = 11.2 Hz, 2H) |
| I-175 | BXA | BXD | 779.2 | 11.09 (s, 1H), 9.77 (s, 1H), 8.89 (s, 1H), 8.42 (s, |
| | | | | 1H), 8.35 (s, 1H), 7.16 (s, 1H), 7.05-6.95 (m, |
| | | | | 3H), 5.38 (dd, J = 6.0, 12.8 Hz, 1H), 4.44-4.34 |
| | | | | (m, 1H), 3.96 (s, 3H), 3.60 (s, 3H), 3.30-3.28 |
| | | | | (m, 4H), 3.11-3.02 (m, 1H), 2.95-2.83 (m, |
| | | | | 1H), 2.77-2.68 (m, 1H), 2.65-2.57 (m, 1H), |
| | | | | 2.18-2.14 (m, 2H), 2.04-1.94 (m, 4H), 1.94- |
| | | | | 1.88 (m, 2H), 1.88-1.79 (m, 4H), 1.76-1.66 |
| | | | | (m, 1H), 1.26-1.07 (m, 3H) |
| I-193 | BXA | BXJ | 769.2 | 11.09 (s, 1H), 9.61 (s, 1H), 9.59-9.56 (m, 1H), |
| | | | | 8.74 (d, J = 1.6 Hz, 1H), 8.40 (d, J = 2.0 Hz, |
| | | | | 1H), 8.29 (s, 1H), 8.00 (s, 1H), 7.07 (s, 1H), 7.05- |
| | | | | 7.00 (m, 2H), 7.00-6.95 (m, 1H), 5.37 (d, J = |
| | | | | 5.6, 12.4 Hz, 1H), 4.43-4.33 (m, 1H), 4.03 (s, |
| | | | | 3H), 3.87 (s, 3H), 3.59 (s, 3H), 3.23-3.21 (m, |
| | | | | 1H), 3.00 (d, J = 10.8 Hz, 2H), 2.94-2.84 (m, |
| | | | | 1H), 2.75-2.60 (m, 2H), 2.23 (d, J = 6.8 Hz, 2H), |
| | | | | 2.18-2.06 (m, 4H), 2.02-1.87 (m, 5H), 1.84-1.75 |
| | | | | (m, 4H), 1.71-1.62 (m, 1H), 1.19-1.08 (m, 2H) |
| I-194 | BXA | BXB | 773.5 | 11.09 (s, 1H), 9.93 (s, 1H), 9.25 (s, 1H), 8.56 (d, |
| | | | | J = 8.0 Hz, 1H), 8.31 (s, 1H), 8.12-8.05 (m, |
| | | | | 2H), 7.09 (s, 1H), 7.04-6.96 (m, 3H), 5.37 (dd, |
| | | | | J = 5.6, 12.8 Hz, 1H), 4.44-4.32 (m, 1H), 3.87 |
| | | | | (s, 3H), 3.59 (s, 3H), 3.01 (d, J = 12.0 Hz, 2H), |
| | | | | 2.95-2.83 (m, 1H), 2.76-2.60 (m, 2H), 2.26- |
| | | | | 2.20 (m, 2H), 2.18-2.07 (m, 4H), 2.05-1.95 |
| | | | | (m, 4H), 1.93-1.86 (m, 2H), 1.84-1.76 (m, |
| | | | | 4H), 1.70-1.62 (m, 1H), 1.20-1.06 (m, 2H) |
| I-196 | BSS | BXK | 739.1 | 11.10 (s, 1H), 9.84 (s, 1H), 9.04 (s, 1H), 8.83 (d, |
| | | | | J = 2.4 Hz, 1H), 8.41 (s, 1H), 8.31 (s, 1H), 8.04 |
| | | | | (s, 1H), 7.07 (s, 1H), 7.06-6.97 (m, 3H), 5.38 |
| | | | | (dd, J = 5.2, 12.4 Hz, 1H), 4.48-4.35 (m, 1H), |
| | | | | 3.87 (s, 3H), 3.61 (s, 3H), 2.97-2.67 (m, 4H), |
| | | | | 2.65-2.57 (m, 2H), 2.52 (s, 2H), 2.18 (d, J = |
| | | | | 10.8 Hz, 2H), 2.05-1.86 (m, 10H), 1.82 (s, 1H), |
| | | | | 1.29-1.15 (m, 3H) |
| I-214 | BUP | BXV | 760.4 | 11.09 (s, 1H), 10.55 (s, 1H), 8.39 (s, 1H), 8.25 |
| | | | | (d, J = 1.2 Hz, 1H), 8.04-7.92 (m, 2H), 7.62- |
| | | | | 7.52 (m, 2H), 7.35 (dd, J = 2.0, 9.2 Hz, 1H), 7.05- |
| | | | | 6.95 (m, 3H), 5.37 (dd, J = 5.2, 12.8 Hz, 1H), |
| | | | | 4.50-4.39 (m, 1H), 3.59 (s, 3H), 3.08 (d, J = |
| | | | | 8.4 Hz, 3H), 2.92-2.84 (m, 1H), 2.72 (d, J = |
| | | | | 4.0 Hz, 1H), 2.64-2.58 (m, 1H), 2.19-2.14 (m, |
| | | | | 4H), 2.03-1.89 (m, 6H), 1.85-1.81 (m, 4H), |
| | | | | 1.73-1.65 (m, 1H), 1.23-1.10 (m, 3H) |
| I-215 | BUP | BXW | 749.3 | 11.1 (s, 1H), 10.8 (s, 1H), 8.85 (s, 1H), 8.41 (s, |
| | | | | 1H), 8.24-8.21 (m, 1H), 7.60 (d, J = 1.2 Hz, |
| | | | | 2H), 7.04-6.96 (m, 3H), 5.37 (dd, J = 5.6, 12.4 |
| | | | | Hz, 1H), 4.49-4.39 (m, 1H), 3.59 (s, 3H), 3.00 |
| | | | | (d, J = 11.2 Hz, 2H), 2.93-2.84 (m, 1H), 2.76- |
| | | | | 2.68 (m, 1H), 2.65-2.59 (m, 1H), 2.22 (d, J = |
| | | | | 7.2 Hz, 2H), 2.16 (d, J = 10.4 Hz, 2H), 2.12-2.06 |
| | | | | (m, 2H), 2.02-1.94 (m, 4H), 1.92-1.88 (m, 1H), |
| | | | | 1.80 (s, 4H), 1.77-1.61 (m, 2H), 1.19-1.09 (m, 2H) |
| I-247 | BUP | CAM | 715.2 | 11.1 (s, 1H), 10.7 (s, 1H), 8.40 (s, 1H), 8.26 (s, |
| | | | | 1H), 8.17-8.13 (m, 1H), 7.59 (d, J = 1.2 Hz, |
| | | | | 2H), 7.04-6.97 (m, 3H), 5.37 (dd, J = 5.2, 12.4 |
| | | | | Hz, 1H), 4.50-4.41 (m, 1H), 3.60 (s, 3H), 2.90- |
| | | | | 2.83 (m, 1H), 2.72 (d, J = 4.4 Hz, 1H), 2.64 (d, |
| | | | | J = 4.0 Hz, 1H), 2.60 (d, J = 1.2 Hz, 1H), 2.44- |
| | | | | 2.36 (m, 4H), 2.20-2.15 (m, 2H), 2.02-1.91 |
| | | | | (m, 6H), 1.90-1.82 (m, 4H), 1.26-1.12 (m, 4H) |
| I-281 | CCQ | CCM | 775.4 | 11.09 (s, 1H), 10.50 (s, 1H), 8.69 (s, 1H), 8.50- |
| | | | | 8.44 (m, 1H), 8.43-8.37 (m, 1H), 8.33 (s, 1H), |
| | | | | 8.22 (dd, J = 0.8, 7.6 Hz, 1H), 7.16 (s, 1H), 7.02- |
| | | | | 6.95 (m, 1H), 6.89 (dd, J = 7.8, 13.2 Hz, 2H), |
| | | | | 5.35 (dd, J = 5.6, 12.8 Hz, 1H), 4.50-4.37 (m, |
| | | | | 1H), 3.98 (s, 3H), 3.64 (s, 3H), 3.62-3.48 (m, |
| | | | | 2H), 3.19-2.97 (m, 2H), 2.91-2.73 (m, 3H), |
| | | | | 2.72-2.57 (m, 2H), 2.19-2.08 (m, 4H), 2.06-1.95 |
| | | | | (m, 4H), 1.94-1.53 (m, 3H), 1.52-1.36 (m, 2H) |
| I-416 | CCJ | CGH | 756.3 | 11.09 (s, 1H), 9.14 (s, 1H), 8.98-8.80 (m, 1H), |
| | | | | 8.44-8.32 (m, 2H), 8.27 (s, 1H), 7.84 (s, 1H), |
| | | | | 7.05-6.83 (m, 3H), 5.42-5.29 (m, 1H), 4.38- |
| | | | | 4.21 (m, 1H), 3.81-3.69 (m, 1H), 3.62 (s, 3H), |
| | | | | 3.03-2.80 (m, 7H), 2.75-2.61 (m, 4H), 2.19 |
| | | | | (d, J = 6.0 Hz, 2H), 2.04-1.95 (m, 1H), 1.94- |
| | | | | 1.80 (m, 4H), 1.58-1.45 (m, 1H), 1.34 (d, J = |
| | | | | 12.8 Hz, 2H), 1.26 (d, J = 6.0 Hz, 6H), 1.07- |
| | | | | 0.89 (m, 2H) |
| I-417 | CEY | CGH | 755.3 | 11.09 (s, 1H), 9.14 (s, 1H), 8.95-8.83 (m, 1H), |
| | | | | 8.42-8.33 (m, 2H), 8.27 (s, 1H), 7.84 (s, 1H), |
| | | | | 7.05-6.93 (m, 3H), 5.37 (dd, J = 5.2, 12.4 Hz, |
| | | | | 1H), 4.42-4.20 (m, 1H), 3.82-3.65 (m, 1H), |
| | | | | 3.63-3.55 (m, 3H), 3.01-2.83 (m, 3H), 2.72- |
| | | | | 2.59 (m, 3H), 2.16 (d, J = 6.8 Hz, 2H), 2.09- |
| | | | | 1.97 (m, 3H), 1.87 (t, J = 12.4 Hz, 4H), 1.82- |
| | | | | 1.70 (m, 4H), 1.58-1.43 (m, 1H), 1.41-1.31 (m, |
| | | | | 2H), 1.26 (d, J = 6.4 Hz, 6H), 1.07-0.89 (m, 2H) |
| I-418 | CGB | CGH | 738.5 | 10.53 (s, 1H), 9.42 (s, 1H), 9.14 (s, 1H), 8.94- |
| | | | | 8.84 (m, 1H), 8.53 (s, 1H), 8.42-8.34 (m, 2H), |
| | | | | 8.27 (s, 1H), 7.84 (s, 1H), 7.76-7.68 (m, 1H), |
| | | | | 7.62 (d, J = 8.4 Hz, 1H), 7.28 (d, J = 7.2 Hz, |
| | | | | 1H), 4.37-4.25 (m, 1H), 3.96-3.85 (m, 1H), |
| | | | | 3.80-3.65 (m, 2H), 3.22-3.05 (m, 4H), 3.02- |
| | | | | 2.91 (m, 1H), 2.81-2.66 (m, 5H), 2.31-2.23 |
| | | | | (m, 2H), 1.96-1.84 (m, 4H), 1.62-1.47 (m, |
| | | | | 1H), 1.44-1.30 (m, 2H), 1.27 (d, J = 6.0 Hz, |
| | | | | 6H), 1.8-0.94 (m, 2H) |

^{a}Coupling was run anywhere from 30 min-2 hrs at rt.

### Example 4 (Method 6): Synthesis of N-[2-[3-[[4-[5-chloro-1-(2,6-dioxo-3-piperidyl)-3-methyl-2-oxo-benzimidazol-4-yl]-1-piperidyl]methyl]cyclobutyl]-6-methoxy-indazol-5-yl]-6-(trifluoromethyl)pyridine-2-carboxamide (I-87)

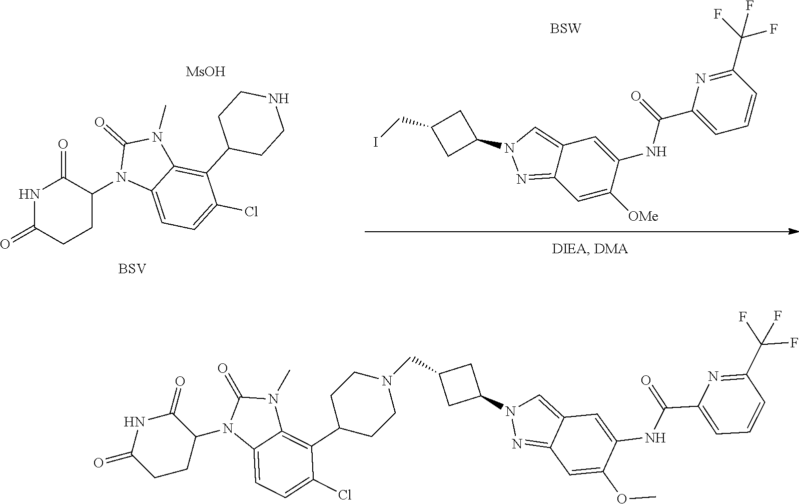

To a mixture of 3-[5-chloro-3-methyl-2-oxo-4-(4-piperidyl)benzimidazol-1-yl]piperidine-2,6-dione (40.0 mg, 106 umol, Intermediate BSV) and N-[2-[3-(iodomethyl)cyclobutyl]-6-methoxy-indazol-5-yl]-6-(trifluoromethyl)pyridine-2-carboxamide (56.2 mg, 106 umol, Intermediate BSW) in DMA (1 mL) was added DIEA (41.1 mg, 318 umol). The reaction mixture was stirred at 70° C. for 24 hours. On completion, the reaction mixture was concentrated in vacuo. The residue was purified by prep-HPLC (column: Welch Xtimate C18 150*25 mm*5 um; mobile phase: [water (0.225% FA)-ACN]; B %: 20%-50%, 11 min) to give the title compound (16.1 mg, 99% purity) as white solid. ¹H NMR (400 MHz, DMSO-d₆) δ 11.12 (s, 1H), 10.52 (s, 1H), 8.70 (s, 1H), 8.48-8.45 (m, 1H), 8.41-8.38 (m, 2H), 8.22 (d, J=7.6 Hz, 1H), 7.20 (s, 1H), 7.12-7.09 (m, 1H), 7.07-7.04 (m, 1H), 5.40-(dd, J=5.2, 13.2 Hz, 1H), 5.22-5.17 (m, 1H), 4.00 (s, 3H), 3.65 (s, 3H), 3.38-3.35 (m, 3H), 2.92-2.83 (m, 2H), 2.77-2.73 (m, 3H), 2.70 (d, J=3.6 Hz, 1H), 2.64 (s, 1H), 2.60 (s, 1H), 2.54-2.52 (m, 3H), 2.44-2.37 (m, 2H), 2.04-1.95 (m, 2H), 1.79-1.67 (m, 2H). LC-MS (ESI⁺) m/z 779.4 (M+H)⁺.

The following compounds in Table 6 where prepared according to the same procedure of Method 6 above.

TABLE 6
Compounds prepared by Method 6.

| | Inter- | Inter- | LCMS | |
|---|---|---|---|---|
| | mediate | mediate | (ES+) m/z | |
| I-# | Amine | Iodide | (M + H)⁺ | 1H NMR (400 MHz, DMSO-d6) δ |
| I-89 | BSP | BSW | 763.4 | 11.10 (s, 1H), 10.54-10.49 (m, 1H), 8.69 (s, 1H), |
| | | | | 8.49-8.45 (m, 1H), 8.42 (d, J = 7.6 Hz, 1H), 8.28 |
| | | | | (s, 1H), 8.22 (d, J = 8.0 Hz, 1H), 7.21 (s, 1H), 6.99 |
| | | | | (dd, J = 4.4, 8.6 Hz, 1H), 6.91-6.82 (m, 1H), 5.41- |
| | | | | 5.34 (m, 1H), 5.24-5.15 (m, 1H), 3.99 (s, 3H), |
| | | | | 3.59 (s, 3H), 3.00 (d, J = 6.4 Hz, 2H), 2.89-2.83 |
| | | | | (m, 1H), 2.75-2.68 (m, 3H), 2.65-2.53 (m, 3H), |
| | | | | 2.35-2.29 (m, 3H), 2.18-2.03 (m, 5H), 2.02-1.97 |
| | | | | (m, 1H), 1.82-1.73 (m, 2H) |
| I-90 | BSY | BSX | 749.4 | 11.09 (s, 1H), 10.53 (s, 1H), 8.64 (s, 1H), 8.58 (s, |
| | | | | 1H), 8.46 (s, 1H), 8.44-8.39 (m, 1H), 8.26-8.23 |
| | | | | (m, 1H), 7.98 (s, 1H), 7.07-6.94 (m, 3H), 5.37 (dd, |
| | | | | J = 5.6, 12.4 Hz, 1H), 5.32-5.26 (m, 1H), 3.59 (s, |
| | | | | 3H), 3.02 (d, J = 9.6 Hz, 2H), 2.94-2.84 (m, 1H), |
| | | | | 2.75-2.70 (m, 3H), 2.69-2.62 (m, 2H), 2.60 (s, |
| | | | | 2H), 2.37 (t, J = 8.4 Hz, 3H), 2.19-2.11 (m, 2H), |
| | | | | 2.04-1.96 (m, 1H), 1.85-1.74 (m, 4H) |
| I-91 | BSZ | BSW | 727.5 | 10.55-10.50 (m, 2H), 9.61 (s, 1H), 8.70 (s, 1H), |
| | | | | 8.57 (s, 1H), 8.49-8.40 (m, 2H), 8.39 (s, 1H), 8.22 |
| | | | | (d, J = 7.6 Hz, 1H), 7.87-7.84 (m, 1H), 7.78 (t, J = |
| | | | | 7.6 Hz, 1H), 7.66 (d, J = 7.6 Hz, 1H), 7.21 (s, |
| | | | | 1H), 5.26-5.17 (m, 1H), 3.99 (s, 3H), 3.92 (ddd, J = |
| | | | | 52, 9.8, 12.4 Hz, 1H), 3.75-3.68 (m, 1H), 3.65- |
| | | | | 3.55 (m, 1H), 3.06 (d, J = 11.6 Hz, 2H), 3.01- |
| | | | | 2.93 (m, 1H), 2.80-2.76 (m, 1H), 2.76-2.70 (m, |
| | | | | 3H), 2.64 (s, 2H), 2.38-2.28 (m, 4H), 1.97 (s, 4H) |
| I-137 | BQM | BSW | 759.4 | 11.08 (s, 1H), 10.51 (s, 1H), 8.70-8.68 (m, 1H), |
| | | | | 8.48-8.45 (m, 1H), 8.43-8.37 (m, 2H), 8.22 (dd, |
| | | | | J = 1.2, 7.6 Hz, 1H), 7.20 (s, 1H), 6.90-6.86 (m, |
| | | | | 1H), 6.84-6.80 (m, 1H), 5.38-5.29 (m, 1H), 5.25- |
| | | | | 5.15 (m, 1H), 3.99 (s, 3H), 3.62 (s, 3H), 3.53- |
| | | | | 3.46 (m, 1H), 3.10-3.04 (m, 2H), 2.91-2.83 (m, |
| | | | | 1H), 2.76-2.68 (m, 4H), 2.65-2.59 (m, 2H), 2.46 |
| | | | | (s, 3H), 2.39-2.34 (m, 2H), 2.30-2.13 (m, 5H), |
| | | | | 2.00-1.94 (m, 1H), 1.74-1.67 (m, 2H) |
| I-138 | BUC | BSW | 775.5 | 11.08 (s, 1H), 10.52 (s, 1H), 8.70 (s, 1H), 8.50- |
| | | | | 8.45 (m, 1H), 8.44-8.38 (m, 2H), 8.23 (dd, J = 0.8, |
| | | | | 8.0 Hz, 1H), 7.22 (s, 1H), 6.94-6.87 (m, 1H), 6.72 |
| | | | | (d, J = 8.8 Hz, 1H), 5.33 (dd, J = 5.2, 12.8 Hz, 1H), |
| | | | | 5.24-5.16 (m, 1H), 4.00 (s, 3H), 3.77 (s, 3H), 3.57 |
| | | | | (s, 3H), 2.99 (d, J = 12.0 Hz, 2H), 2.93-2.83 (m, |
| | | | | 1H), 2.74-2.66 (m, 4H), 2.62-2.54 (m, 3H), 2.44- |
| | | | | 2.31 (m, 5H), 2.10-2.02 (m, 2H), 2.01-1.95 (m, |
| | | | | 1H), 1.58 (d, J = 10.8 Hz, 2H) |
| I-139 | AQK | BUF | 794.6 | 11.09 (s, 1H), 10.50 (s, 1H), 8.61 (s, 1H), 8.50 (s, |
| | | | | 1H), 8.48-8.38 (m, 2H), 8.24-8.21 (m, 1H), 8.02 |
| | | | | (s, 1H), 7.40-7.11 (m, 1H), 7.01-6.95 (m, 1H), |
| | | | | 6.94-6.90 (m, 1H), 6.87 (d, J = 7.6 Hz, 1H), 5.40- |
| | | | | 5.30 (m, 2H), 3.66 (s, 3H), 3.18 (d, J = 11.2 Hz, |
| | | | | 2H), 2.94-2.85 (m, 1H), 2.78-2.70 (m, 4H), 2.69-2.66 |
| | | | | (m, 3H), 2.66-2.58 (m, 2H), 2.52 (d, J = 2.0 Hz, |
| | | | | 1H), 2.40-2.34 (m, 2H), 2.27 (s, 3H), 2.03-1.96 (m, |
| | | | | 1H), 1.86 (d, J = 10.4 Hz, 2H), 1.77-1.67 (m, 2H) |
| I-140 | AZK | BUJ | 715.1 | 11.09 (s, 1H), 10.37 (s, 1H), 8.45 (s, 1H), 8.43- |
| | | | | 8.33 (m, 2H), 8.30 (d, J = 1.2 Hz, 1H), 8.18 (dd, J = |
| | | | | 1.2, 7.6 Hz, 1H), 7.69-7.62 (m, 1H), 7.60-7.54 |
| | | | | (m, 1H), 7.06-6.94 (m, 3H), 5.42-5.33 (m, 1H), |
| | | | | 5.31-5.22 (m, 1H), 3.59 (s, 3H), 3.26-3.19 (m, |
| | | | | 2H), 3.02 (d, J = 10.8 Hz, 2H), 2.95-2.83 (m, 1H), |
| | | | | 2.76-2.70 (m, 3H), 2.66-2.62 (m, 1H), 2.59 (d, J = |
| | | | | 5.2 Hz, 2H), 2.36 (t, J = 8.4 Hz, 2H), 2.14 (dt, J = |
| | | | | 3.2, 11.2 Hz, 2H), 2.03-1.95 (m, 1H), 1.85-1.72 (m, 4H) |
| I-141 | BVK | BSW | 763.4 | 11.10 (s, 1H), 10.54-10.49 (m, 1H), 8.69 (s, 1H), |
| | | | | 8.49-8.45 (m, 1H), 8.42 (d, J = 7.6 Hz, 1H), 8.28 |
| | | | | (s, 1H), 8.22 (d, J = 8.0 Hz, 1H), 7.21 (s, 1H), 6.99 |
| | | | | (dd, J = 4.4, 8.6 Hz, 1H), 6.91-6.82 (m, 1H), 5.41- |
| | | | | 5.34 (m, 1H), 5.24-5.15 (m, 1H), 3.99 (s, 3H), |
| | | | | 3.59 (s, 3H), 3.00 (d, J = 6.4 Hz, 2H), 2.89-2.83 |
| | | | | (m, 1H), 2.75-2.68 (m, 3H), 2.65-2.53 (m, 3H), |
| | | | | 2.35-2.29 (m, 3H), 2.18-2.03 (m, 5H), 2.02-1.97 |
| | | | | (m, 1H), 1.82-1.73 (m, 2H) |
| I-142 | BSP | BSX | 767.4 | 11.09 (s, 1H), 10.53 (s, 1H), 8.66 (s, 1H), 8.59 (d, |
| | | | | J = 0.4 Hz, 1H), 8.47-8.40 (m, 1H), 8.38 (s, 1H), |
| | | | | 8.22 (dd, J = 1.2, 7.6 Hz, 1H), 7.97 (s, 1H), 7.85 (s, |
| | | | | 1H), 7.04 (dd, J = 4.2, 8.7 Hz, 1H), 6.91 (dd, J = |
| | | | | 8.8, 12.8 Hz, 1H), 5.37 (dd, J = 5.2, 12.6 Hz, 1H), |
| | | | | 5.32-5.24 (m, 1H), 3.64 (s, 3H), 3.55 (d, J = 12.4 |
| | | | | Hz, 2H), 3.40 (d, J = 7.2 Hz, 2H), 3.20 (t, J = 12.2 |
| | | | | Hz, 2H), 3.12-3.01 (m, 1H), 2.94-2.79 (m, 3H), |
| | | | | 2.74-2.63 (m, 2H), 2.62-2.54 (m, 2H), 2.41-2.28 (m, |
| | | | | 2H), 2.06 (d, J = 13.6 Hz, 2H), 2.02-1.95 (m, 1H) |
| I-143 | BQM | BSX | 763.4 | 11.08 (s, 1H), 10.53 (s, 1H), 8.64 (s, 1H), 8.58 (s, |
| | | | | 1H), 8.49 (d, J = 7.6 Hz, 1H), 8.44-8.39 (m, 1H), |
| | | | | 8.24 (d, J = 7.6 Hz, 1H), 7.98 (s, 1H), 6.89-6.85 |
| | | | | (m, 1H), 6.84-6.80 (m, 1H), 5.37-5.26 (m, 2H), |
| | | | | 3.61 (s, 3H), 3.51-3.45 (m, 1H), 3.05-3.00 (m, |
| | | | | 2H), 2.90-2.83 (m, 1H), 2.76-2.68 (m, 4H), 2.65- |
| | | | | 2.61 (m, 1H), 2.60-2.57 (m, 2H), 2.46 (s, 3H), |
| | | | | 2.40-2.36 (m, 2H), 2.23 (m, 2H), 2.13-2.07 (m, |
| | | | | 2H), 2.00-1.94 (m, 1H), 1.72-1.66 (m, 2H) |
| I-144 | BQL | BSX | 783.4 | 11.12 (s, 1H), 10.53 (s, 1H), 8.64 (s, 1H), 8.58 (s, |
| | | | | 1H), 8.51-8.45 (m, 1H), 8.45-8.38 (m, 1H), 8.24 |
| | | | | (dd, J = 0.8, 7.6 Hz, 1H), 8.18 (s, 1H), 7.98 (s, 1H), |
| | | | | 7.12-7.06 (m, 1H), 7.05-7.00 (m, 1H), 5.39 (dd, |
| | | | | J = 5.2, 12.4 Hz, 1H), 5.29 (s, 1H), 3.63 (s, 3H), |
| | | | | 3.49 (dd, J = 3.6, 12.0 Hz, 1H), 3.02 (d, J = 11.2 |
| | | | | Hz, 2H), 2.92-2.83 (m, 1H), 2.75-2.69 (m, 3H), |
| | | | | 2.68-2.62 (m, 2H), 2.59 (s, 3H), 2.37 (t, J = 8.0 |
| | | | | Hz, 2H), 2.11 (t, J = 11.2 Hz, 2H), 2.05-1.96 (m, |
| | | | | 1H), 1.65 (d, J = 10.0 Hz, 2H) |
| I-145 | BUC | BSX | 779.5 | 11.07 (s, 1H), 10.53 (s, 1H), 8.64 (s, 1H), 8.58 (s, |
| | | | | 1H), 8.49-8.45 (m, 1H), 8.45-8.39 (m, 1H), 8.26- |
| | | | | 8.23 (m, 1H), 8.19 (s, 1H), 7.98 (s, 1H), 6.91 (d, |
| | | | | J = 8.4 Hz, 1H), 6.72 (d, J = 8.8 Hz, 1H), 5.35- |
| | | | | 5.25 (m, 2H), 3.76 (s, 3H), 3.56 (s, 3H), 2.99 (d, J = |
| | | | | 11.2 Hz, 2H), 2.92-2.83 (m, 1H), 2.76-2.61 (m, 5H), |
| | | | | 2.59 (s, 2H), 2.45-2.35 (m, 5H), 2.07 (t, J = 11.2 Hz, |
| | | | | 2H), 2.01-1.93 (m, 1H), 1.58 (d, J = 11.6 Hz, 2H) |
| I-146 | BQM | BUU | 730.2 | 11.07 (s, 1H), 10.21 (s, 1H), 9.10 (s, 1H), 8.65 (s, |
| | | | | 1H), 8.54-8.45 (m, 2H), 8.42 (t, J = 8.0 Hz, 1H), |
| | | | | 8.24 (d, J = 7.6 Hz, 1H), 6.91-6.79 (m, 2H), 5.43- |
| | | | | 5.29 (m, 2H), 3.62 (s, 3H), 3.53-3.41 (m, 2H), |
| | | | | 3.07-3.01 (m, 2H), 2.92-2.82 (m, 1H), 2.80-2.70 |
| | | | | (m, 3H), 2.68-2.57 (m, 4H), 2.46 (s, 3H), 2.29- |
| | | | | 2.21 (m, 2H), 2.21-2.07 (m, 3H), 2.02-1.93 (m, |
| | | | | 1H), 1.74-1.64 (m, 2H) |
| I-147 | BSP | BUU | 734.2 | 11.10 (s, 1H), 10.21 (s, 1H), 9.10 (s, 1H), 8.65 (s, |
| | | | | 1H), 8.51-8.46 (m, 2H), 8.45-8.39 (m, 1H), 8.23 |
| | | | | (dd, J = 0.8, 7.6 Hz, 1H), 6.99 (dd, J = 4.4, 8.4 Hz, |
| | | | | 1H), 6.86 (dd, J = 8.4, 12.9 Hz, 1H), 5.41-5.34 (m, |
| | | | | 2H), 3.59 (s, 3H), 3.01 (d, J = 6.0 Hz, 2H), 2.92- |
| | | | | 2.83 (m, 1H), 2.79-2.73 (m, 3H), 2.71 (d, J = 4.4 |
| | | | | Hz, 1H), 2.65-2.57 (m, 3H), 2.52 (d, J = 2.0 Hz, |
| | | | | 1H), 2.41 (t, J = 8.4 Hz, 2H), 2.12 (s, 4H), 1.99 (td, |
| | | | | J = 5.2, 10.4 Hz, 1H), 1.77 (d, J = 3.4 Hz, 2H) |
| I-148 | BQM | BUJ | 729.5 | 11.08 (s, 1H), 10.38 (s, 1H), 8.46 (s, 1H), 8.43- |
| | | | | 8.33 (m, 2H), 8.31 (d, J = 1.2 Hz, 1H), 8.18 (dd, J = |
| | | | | 1.2, 7.5 Hz, 1H), 7.68-7.63 (m, 1H), 7.60-7.55 |
| | | | | (m, 1H), 6.98-6.73 (m, 2H), 5.39-5.20 (m, 2H), |
| | | | | 3.62 (s, 3H), 3.52-3.41 (m, 1H), 3.03 (d, J = 10.8 |
| | | | | Hz, 2H), 2.90-2.82 (m, 1H), 2.76-2.69 (m, 3H), |
| | | | | 2.67 (dd, J = 1.6, 3.6 Hz, 1H), 2.59 (d, J = 6.0 Hz, |
| | | | | 2H), 2.46 (s, 3H), 2.42-2.32 (m, 3H), 2.27-2.17 |
| | | | | (m, 2H), 2.17-2.04 (m, 2H), 2.01-1.94 (m, 1H), |
| | | | | 1.69 (d, J = 11.2 Hz, 2H) |
| I-149 | BSV | BUU | 750.5 | 11.12 (s, 1H), 10.21 (s, 1H), 9.10 (s, 1H), 8.65 (s, |
| | | | | 1H), 8.50-8.47 (m, 2H), 8.42 (t, J = 7.6 Hz, 1H), |
| | | | | 8.25-8.22 (m, 1H), 7.11-7.07 (m, 1H), 7.05-7.02 |
| | | | | (m, 1H), 5.42-5.36 (m, 2H), 3.63 (s, 3H), 3.53- |
| | | | | 3.47 (m, 1H), 3.02 (d, J = 10.4 Hz, 2H), 2.93-2.83 |
| | | | | (m, 1H), 2.77-2.72 (m, 3H), 2.68-2.62 (m, 3H), |
| | | | | 2.60 (s, 3H), 2.43-2.39 (m, 2H), 2.11 (t, J = 10.8 |
| | | | | Hz, 2H), 2.03-1.98 (m, 1H), 1.65 (d, J = 11.6 Hz, 2H) |
| I-150 | BQL | BUJ | 749.1 | 11.12 (s, 1H), 10.37 (s, 1H), 8.45 (s, 1H), 8.42- |
| | | | | 8.34 (m, 2H), 8.30 (s, 1H), 8.23-8.15 (m, 1H), 7.68- |
| | | | | 7.62 (m, 1H), 7.60-7.54 (m, 1H), 7.16-6.99 (m, |
| | | | | 2H), 5.44-5.18 (m, 2H), 3.63 (s, 3H), 3.54-3.49 |
| | | | | (m, 1H), 3.02 (d, J = 10.4 Hz, 2H), 2.89-2.83 (m, |
| | | | | 1H), 2.72 (d, J = 4.4 Hz, 3H), 2.65 (d, J = 11.6 Hz, |
| | | | | 2H), 2.59 (s, 2H), 2.40-2.31 (m, 4H), 2.10 (t, J = 10.8 |
| | | | | Hz, 2H), 2.02-1.97 (m, 1H), 1.65 (d, J = 12.0 Hz, 2H) |
| I-151 | BVK | BUJ | 733.5 | 11.10 (s, 1H), 10.37 (s, 1H), 8.45 (s, 1H), 8.42- |
| | | | | 8.38 (m, 1H), 8.38-8.33 (m, 1H), 8.30 (s, 1H), 8.17 |
| | | | | (dd, J = 1.2, 7.6 Hz, 1H), 7.68-7.63 (m, 1H), 7.68 |
| | | | | (dd, J = 1.6, 8.8 Hz, 1H), 7.03-6.96 (m, 1H), 6.90- |
| | | | | 6.82 (m, 1H), 5.41-5.33 (m, 1H), 5.3-5.21 (m, |
| | | | | J = 6.8 Hz, 1H), 3.59 (s, 3H), 3.05-2.96 (m, 2H), |
| | | | | 2.92-2.82 (m, 1H), 2.75-2.69 (m, 3H), 2.69-2.61 |
| | | | | (m, 2H), 2.60-2.56 (m, 2H), 2.52 (s, 1H), 2.40- |
| | | | | 2.35 (m, 2H), 2.15-2.05 (m, 4H), 2.04-1.95 (m, |
| | | | | 1H), 1.82-1.74 (m, 2H) |
| I-152 | BVA | BUJ | 745.2 | 11.08 (s, 1H), 10.38 (s, 1H), 8.47 (s, 1H), 8.44- |
| | | | | 8.35 (m, 2H), 8.31 (d, J = 1.2 Hz, 1H), 8.23-8.16 |
| | | | | (m, 1H), 7.70-7.64 (m, 1H), 7.58 (dd, J = 2.0, 9.2 |
| | | | | Hz, 1H), 6.91 (d, J = 8.4 Hz, 1H), 6.73 (d, J = 8.8 |
| | | | | Hz, 1H), 5.39-5.22 (m, 2H), 3.77 (s, 3H), 3.57 (s, |
| | | | | 3H), 2.99 (d, J = 10.4 Hz, 2H), 2.94-2.83 (m, 1H), |
| | | | | 2.80-2.64 (m, 5H), 2.59 (d, J = 5.6 Hz, 2H), 2.44- |
| | | | | 2.33 (m, 5H), 2.12-1.95 (m, 3H), 1.59 (d, J = |
| | | | | 11.6 Hz, 2H) |
| I-235 | AZK | CAD | 762.3 | 11.09 (s, 1H), 10.52 (s, 1H), 9.02 (s, 1H), 8.55- |
| | | | | 8.38 (m, 2H), 8.29-8.20 (m, 2H), 7.76 (s, 1H), 7.10- |
| | | | | 6.90 (m, 3H), 4.05 (s, 3H), 4.01-3.89 (m, 1H), |
| | | | | 3.59 (s, 3H), 2.99 (d, J = 11.6 Hz, 4H), 2.76-2.62 |
| | | | | (m, 4H), 2.36-2.23 (m, 4H), 2.13 (d, J = 2.4 Hz, |
| | | | | 4H), 1.79 (d, J = 1.6 Hz, 4H) |
| I-270 | AZK | BUU | 716.5 | 11.09 (s, 1H), 10.21 (s, 1H), 9.10 (s, 1H), 8.65 (s, |
| | | | | 1H), 8.52-8.46 (m, 2H), 8.42 (t, J = 7.6 Hz, 1H), |
| | | | | 8.26-8.20 (m, 1H), 7.07-6.93 (m, 3H), 5.43-5.34 |
| | | | | (m, 2H), 3.59 (s, 3H), 3.21 (s, 1H), 3.02 (d, J = 10.4 |
| | | | | Hz, 2H), 2.91-2.84 (m, 1H), 2.80-2.68 (m, 4H), |
| | | | | 2.65-2.57 (m, 3H), 2.42-2.37 (m, 2H), 2.19-2.10 |
| | | | | (m, 2H), 2.03-1.96 (m, 1H) |
| I-292^{b} | CEL | CEI | 801.4 | 11.09 (s, 1H), 10.51 (s, 1H), 8.69 (s, 1H), 8.50- |
| | | | | 8.44 (m, 1H), 8.44-8.37 (m, 1H), 8.34 (s, 1H), 8.22 |
| | | | | (d, J = 7.6 Hz, 1H), 7.17 (s, 1H), 7.09-6.89 (m, |
| | | | | 3H), 5.37 (dd, J = 5.2, 12.8 Hz, 1H), 4.44-4.31 (m, |
| | | | | 1H), 3.98 (s, 3H), 3.62 (s, 3H), 3.57-3.46 (m, 1H), |
| | | | | 2.96-2.79 (m, 2H), 2.78-2.69 (m, 1H), 2.62 (d, J = |
| | | | | 17.2 Hz, 2H), 2.24-2.13 (m, 3H), 2.05-1.86 (m, |
| | | | | 4H), 1.86-1.72 (m, 4H), 1.67 (dd, J = 1.2, 11.6 Hz, |
| | | | | 1H), 1.60-1.44 (m, 2H), 1.26-1.13 (m, 2H), 1.08 |
| | | | | (d, J = 18.0 Hz, 6H) |
| I-305^{c} | CER | CES | 786.3 | 11.10 (s, 1H), 10.13 (s, 1H), 8.53 (s, 1H), 8.45- |
| | | | | 8.35 (m, 3H), 8.22-8.21 (m, 1H), 7.52 (s, 1H), 7.10- |
| | | | | 7.03 (m, 1H), 7.02-6.94 (m, 2H), 5.39 (dd, J = |
| | | | | 4.4, 12.4 Hz, 1H), 4.54-4.42 (m, 1H), 3.66 (s, 3H), |
| | | | | 3.64-3.55 (m, 2H), 3.33-3.26 (m, 3H), 3.14-3.09 |
| | | | | (m, 1H), 3.06-2.96 (m, 1H), 2.96-2.79 (m, 2H), |
| | | | | 2.78-2.63 (m, 2H), 2.41 (s, 3H), 2.30-2.15 (m, |
| | | | | 3H), 2.09-1.84 (m, 5H), 1.51 (s, 3H), 1.45 (s, 3H), |
| | | | | 1.41-1.23 (m, 2H) |
| I-306^{c} | CER | CEI | 802.3 | 11.10 (s, 1H), 10.50 (s, 1H), 8.69 (s, 1H), 8.49- |
| | | | | 8.44 (m, 1H), 8.43-8.38 (m, 1H), 8.35 (s, 1H), 8.22 |
| | | | | (d, J = 7.6 Hz, 1H), 7.15 (s, 1H), 7.08-6.88 (m, |
| | | | | 3H), 5.43-5.31 (m, 1H), 4.54-4.27 (m, 1H), 3.98 |
| | | | | (s, 3H), 3.67 (s, 3H), 3.62 (d, J = 4.0 Hz, 1H), 3.20- |
| | | | | 2.97 (m, 2H), 2.96-2.79 (m, 2H), 2.73-2.64 (m, |
| | | | | 3H), 2.20 (s, 3H), 2.09-1.73 (m, 6H), 1.66-1.19 |
| | | | | (m, 6H), 1.10 (s, 4H) |
| I-324^{d} | CBC | CBB | 772.2 | 11.09 (s, 1H), 10.36 (s, 1H), 8.42-8.33 (m, 3H), |
| | | | | 8.29 (d, J = 1.2 Hz, 1H), 8.17 (dd, J = 1.2, 7.6 Hz, |
| | | | | 1H), 7.64-7.59 (m, 1H), 7.57-7.51 (m, 1H), 7.02- |
| | | | | 6.84 (m, 3H), 5.36 (dd, J = 5.2, 12.8 Hz, 1H), 4.45 |
| | | | | (t, J = 11.6 Hz, 1H), 3.68 (s, 3H), 3.16-3.08 (m, |
| | | | | 1H), 2.94-2.84 (m, 1H), 2.80-2.58 (m, 7H), 2.24-2.12 |
| | | | | (m, 3H), 2.02-1.77 (m, 5H), 1.59-1.48 (m, 1H), 1.46- |
| | | | | 1.23 (m, 2H), 1.10 (s, 6H), 0.86 (t, J = 7.2 Hz, 1H) |
| I-421 | BVA | BUU | 746.3 | 11.08 (s, 1H), 10.21 (s, 1H), 9.11 (s, 1H), 8.65 (s, |
| | | | | 1H), 8.52-8.46 (m, 2H), 8.42 (t, J = 8.0 Hz, 1H), |
| | | | | 8.24 (d, J = 8.0 Hz, 1H), 6.90 (d, J = 8.8 Hz, 1H), |
| | | | | 6.72 (d, J = 8.8 Hz, 1H), 5.43-5.27 (m, 2H), 3.76 |
| | | | | (s, 3H), 3.56 (s, 3H), 3.03-2.94 (m, 2H), 2.93- |
| | | | | 2.82 (m, 1H), 2.79-2.72 (m, 3H), 2.71-2.57 (m, |
| | | | | 4H), 2.44-2.35 (m, 4H), 2.13-2.02 (m, 2H), 2.01- |
| | | | | 1.92 (m, 1H), 1.61-1.54 (m, 2H), 1.46-1.31 (m, 1H) |
| I-424^{b} | CHA | CEI | 801.3 | 11.09 (s, 1H), 10.85 (s, 1H), 10.51 (s, 1H), 8.69 (s, |
| | | | | 1H), 8.49-8.44 (m, 1H), 8.44-8.38 (m, 1H), 8.34 |
| | | | | (s, 1H), 8.22 (d, J = 7.6 Hz, 1H), 7.17 (s, 1H), 7.06- |
| | | | | 6.79 (m, 3H), 5.37 (dd, J = 5.2, 12.4 Hz, 1H), 4.44- |
| | | | | 4.31 (m, 1H), 3.98 (s, 3H), 3.62 (s, 2H), 3.54- |
| | | | | 3.53 (s, 2H), 2.98-2.68 (m, 3H), 2.63-2.61 (m, |
| | | | | 2H), 2.23-2.12 (m, 3H), 2.04-1.96 (m, 1H), 1.95- |
| | | | | 1.73 (m, 6H), 1.71-1.61 (m, 1H), 1.60-1.44 (m, |
| | | | | 2H), 1.25-0.96 (m, 9H) |

^{a}The coupling was run at 60-90° C. for 12-32 hrs.
^{b}K₂CO₃ used as the base for the coupling with 4 Å molecular sieves in DMA at 90° C. for 16 hrs.
^{c}TEA and KI in DMA was stirred at 90° C. for 16 hrs. The coupled product was deprotected with TfOH in TFA at 70° C. for 2 hrs, then purified via prep HPLC to give the final cmpd.
^{d}Cs₂CO₃ in DMF was used for the coupling at 60° C. for 16 hrs.

### Example 5 (Method 7): 6-cyano-N-[2-[4-[[4-[1-(2,6-dioxo-3-piperidyl)-3-methyl-2-oxo-benzimidazol-4-yl]-1-1-piperidyl]methyl]cyclohexyl]-6-methoxy-indazol-5-yl]pyrazolo[1,5-a]pyrimidine-3-carboxamide (I-153)

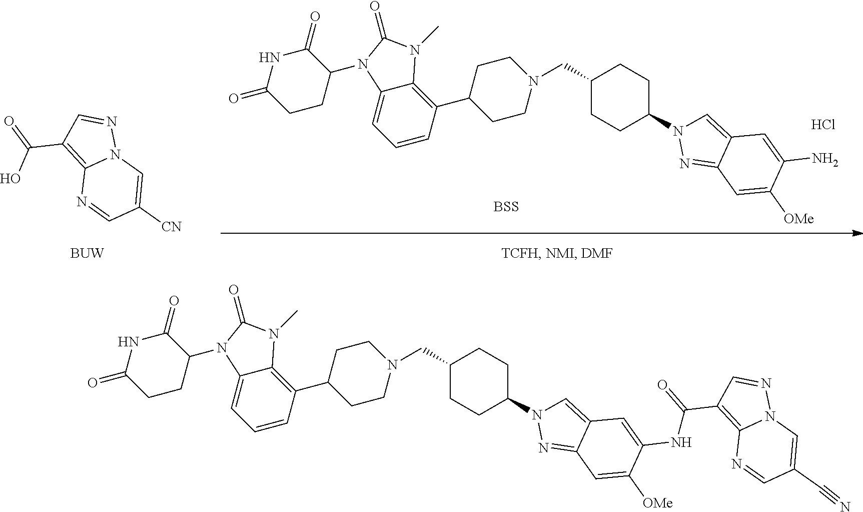 [SMILES: N#Cc1cnc2c(C(=O)O)cnn2c1 ; COc1cc2nn([C@H]3CC[C@H](CN4CCC(c5cccc6c5n(C)c(=O)n6C5CCC(=O)NC5=O)CC4)CC3)cc2cc1N ; "BSS" ; Cl ; "BUW" ; COc1cc2nn([C@H]3CC[C@H](CN4CCC(c5cccc6c5n(C)c(=O)n6C5CCC(=O)NC5=O)CC4)CC3)cc2cc1NC(=O)c1cnn2cc(C#N)cnc12]

To a solution of 3-[4-[1-[[4-(5-amino-6-methoxy-indazol-2-yl)cyclohexyl]methyl]-4-piperidyl]-3-methyl-2-oxo-benzimidazol-1-yl]piperidine-2,6-dione (35.0 mg, 55.0 umol, HCl, Intermediate BSS) in DMF (2 mL) was added 6-cyanopyrazolo[1,5-a]pyrimidine-3-carboxylic acid (10.3 mg, 55.0 umol, Intermediate BUW) and 1-methylimidazole (13.5 mg, 165 umol). Then [chloro(dimethylamino)methylene]-dimethyl-ammonium; hexafluorophosphate (18.5 mg, 66.0 umol) in DMF (1 mL) was added at 0° C. Next, the mixture was stirred at 20° C. for 2 hrs. On completion, the mixture was quenched with H₂O 0.5 mL and purified by pre-HPLC (column: Phenomenex luna C18 150*25 mm*10 um; mobile phase: [water(0.225% FA)-ACN]; B %: 10%-40%, 11.5 min) to give the title compound (7.32 mg, 9.26 umol, 16% yield) as yellow solid. ¹H NMR (400 MHz, DMSO-d₆) δ 11.11 (s, 1H), 10.43 (s, 1H), 10.25 (d, J=2.0 Hz, 1H), 9.28 (d, J=2.0 Hz, 1H), 8.92 (s, 1H), 8.68 (s, 1H), 8.31 (s, 1H), 7.11 (s, 1H), 7.09-6.94 (m, 3H), 5.44-5.34 (m, 1H), 4.49-4.36 (m, 1H), 4.04 (s, 3H), 3.63 (s, 6H), 3.07-2.85 (m, 3H), 2.77-2.65 (m, 2H), 2.24-2.17 (m, 2H), 2.14-1.83 (m, 12H), 1.36-1.22 (m, 2H). LC-MS (ESI⁺) m/z 770.3 (M+H)⁺.

TABLE 5
Compounds synthesized via Method 7, with the corresponding amine and acid.

| | Inter- | Inter- | LCMS | |
|---|---|---|---|---|
| | mediate | mediate | (ES+) m/z | |
| I-# | Amine | Acid | (M + H)+ | 1HNMR (400 MHz, DMSO-d6) δ |
| I-154 | BYE | BUW | 758.3 | 11.09 (s, 1H), 10.25 (d, J = 2.0 Hz, 1H), 9.97 |
| | | | | (d, J = 3.2 Hz, 1H), 9.23 (d, J = 2.0 Hz, 1H), |
| | | | | 8.95 (s, 1H), 8.63-8.58 (m, 1H), 8.47 (s, 1H), |
| | | | | 7.55 (d, J = 12.0 Hz, 1H), 7.07-6.93 (m, 3H), |
| | | | | 5.37 (dd, J = 6.0, 13.2 Hz, 1H), 4.51-4.37 (m, |
| | | | | 1H), 3.59 (s, 3H), 3.22-3.19 (m, 1H), 3.05- |
| | | | | 2.95 (m, 2H), 2.94-2.82 (m, 1H), 2.78-2.68 |
| | | | | (m, 1H), 2.66-2.57 (m, 1H), 2.25-2.20 (m, |
| | | | | 2H), 2.19-2.12 (m, 2H), 2.12-2.03 (m, 2H), |
| | | | | 2.03-1.95 (m, 3H), 1.95-1.86 (m, 2H), 1.85-1.73 |
| | | | | (m, 4H), 1.71-1.61 (m, 1H), 1.21-1.06 (m, 2H) |
| I-344^{a} | AML | AMY | 869.3 | 1.48 (J = 3.20 Hz, 1 H) 1.55-1.71 (m, 12 H) |
| | | | | 1.73-1.90 (m, 5 H) 1.97-2.09 (m, 4 H) 2.11- |
| | | | | 2.20 (m, 2 H) 2.57-2.63 (m, 3 H) 2.73-2.81 |
| | | | | (m, 1 H) 2.83-2.95 (m, 1 H) 3.45-3.55 (m, 2 |
| | | | | H) 4.12-4.25 (m, 1 H) 4.41-4.54 (m, 1 H) |
| | | | | 5.06 (dd, J = 12..4, 5.6 Hz, 1 H) 5.93 (s, 1 H) |
| | | | | 6.49 (d, J = 6.0 Hz, 1 H) 6.96-7.13 (m, 2 H) |
| | | | | 7.51-7.66 (m, 2 H) 8.16 (d, J = 7, 2 Hz, 1 H) |
| | | | | 8.31-8.39 (m, 2 H) 8.42-8.49 (m, 1 H) 8.71 |
| | | | | (s, 1 H) 11.09 (s, 1 H) 12.35 (s, 1 H) |
| I-346^{a} | AML | AXQ | 841.3 | 11.09 (s, 1H), 10.50 (s, 1H), 8.69 (s, 1H), 8.53- |
| | | | | 8.36 (m, 2H), 8.32 (d, J = 2.1 Hz, 1H), 8.22 |
| | | | | (d, J = 7.6 Hz, 1H), 7.65-7.53 (m, 1H), 7.14 |
| | | | | (s, 1H), 7.10-6.99 (m, 2H), 6.49 (d, J = 6.6 Hz, |
| | | | | 1H), 5.10-5.03 (m, 1H), 4.52-4.33 (m, 1H), |
| | | | | 4.25-4.12 (m, 1H), 3.98 (s, 3H), 3.57-3.34 |
| | | | | (m, 4H), 2.97-2.83 (m, 1H), 2.80-2.70 (m, |
| | | | | 1H), 2.64-2.54 (m, 2H), 2.20-2.11 (m, 3H), |
| | | | | 2.06-1.99 (m, 3H), 1.90-1.73 (m, 5H), 1.70-1.63 |
| | | | | (m, 2H), 1.62-1.55 (m, 3H), 1.53-1.45 (m, 1H) |

^{a}ACN was used as the solvent for the coupling.

### Examples 6: Synthesis of N-[2-[4-[[[4-[1-(2,6-dioxo-3-piperidyl)-3-methyl-2-oxo-benzimidazol-4-yl]cyclohexyl]-methyl-amino]methyl]cyclohexyl]-6-methoxy-indazol-5-yl]-6-(trifluoromethyl)pyridine-2-carboxamide (1-5) and N-[2-[4-[[[4-[1-(2,6-dioxo-3-piperidyl)-3-methyl-2-oxo-benzimidazol-4-yl]cyclohexyl]-methyl-amino]methyl]cyclohexyl]-6-methoxy-indazol-5-yl]-6-(trifluoromethyl)pyridine-2-carboxamide (I-6)

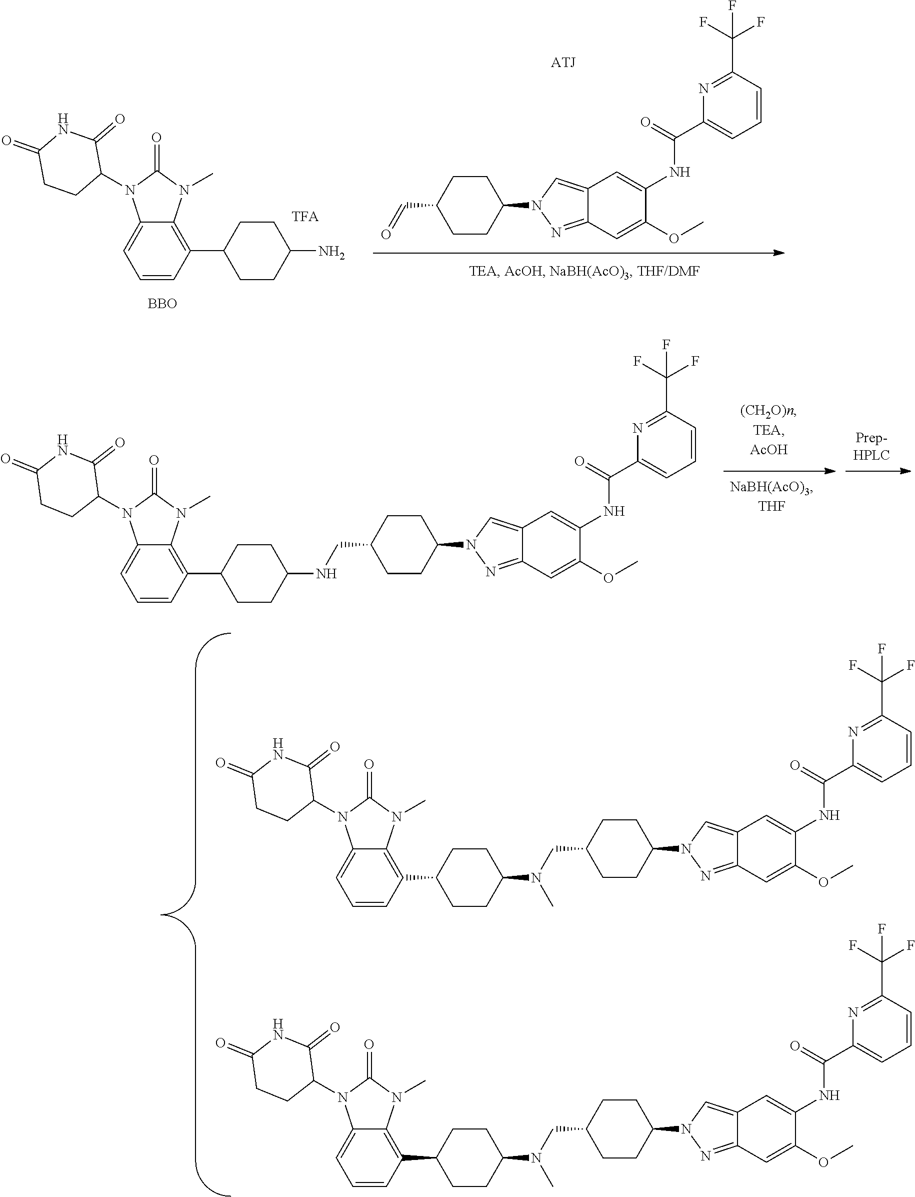

### Step 1—N-[2-[4-[[[4-[1-(2,6-dioxo-3-piperidyl)-3-methyl-2-oxo-benzimidazol-4-yl]cyclohexyl]amino] methyl]cyclohexyl]-6-methoxy-indazol-5-yl]-6-(trifluoromethyl)pyridine-2-carboxamide

To a solution of 3-[4-(4-aminocyclohexyl)-3-methyl-2-oxo-benzimidazol-1-yl]piperidine-2,6-dione (790 mg, 1.68 mmol, TFA, Intermediate BBO) in THF (5 mL) and DMF (1 mL) was added TEA (113 mg, 1.12 mmol). Then HOAc (134 mg, 2.24 mmol) and N-[2-(4-formylcyclohexyl)-6-methoxy-indazol-5-yl]-6-(trifluoromethyl)pyridine-2-carboxamide (0.5 g, 1.12 mmol, Intermediate ATJ) was added at 0° C., then the mixture was stirred at −10° C. for 0.5 hr. Next, NaBH(OAc)₃ (712 mg, 3.36 mmol) was added and the mixture was stirred at −10° C. for 1 hr. On completion, the reaction mixture was quenched by addition water (0.1 mL) and concentrated in vacuo. The crude product was purified by reversed phase flash (0.1% FA condition) to give the title compound (0.85 g, 86% yield) as white solid. LC-MS (ESI⁺) m/z 787.4 (M+H)⁺.

### Step 2—N-[2-[4-[[[4-[1-(2,6-dioxo-3-piperidyl)-3-methyl-2-oxo-benzimidazol-4-yl]cyclohexyl]-methyl-amino]methyl]cyclohexyl]-6-methoxy-indazol-5-yl]-6-(trifluoromethyl)pyridine-2-carboxamide and N-[2-[4-[[[4-[1-(2,6-dioxo-3-piperidyl)-3-methyl-2-oxo-benzimidazol-4-yl]cyclohexyl]-methyl-amino]methyl]cyclohexyl]-6-methoxy-indazol-5-yl]-6-(trifluoromethyl)pyridine-2-carboxamide

A solution of N-[2-[4-[[[4-[1-(2,6-dioxo-3-piperidyl)-3-methyl-2-oxo-benzimidazol-4-yl]cyclohexyl]amino]methyl]cyclohexyl]-6-methoxy-indazol-5-yl]-6-(trifluoromethyl)pyridine-2-carboxamide (0.8 g, 1.02 mmol), TEA (10.2 mg, 101 umol), HOAc (12.2 mg, 203 umol) and (HCHO)n (0.04 g, 10.1 mmol) in THF (5 mL) was stirred at 25° C. for 12 hr. Then NaBH(OAc)₃ (430 mg, 2.03 mmol) was added and the mixture was stirred at 25° C. for 12 hr. On completion, the reaction mixture was quenched by addition water (0.5 mL) and concentrated in vacuo. The residue was purified by prep-HPLC (column: Phenomenex luna C18 150*40 mm*15 um; mobile phase: [water (0.225% FA)-ACN]; B %: 22%-52%, 10 min) and prep-HPLC(column: Waters Vinridis Silica 2-EP OBD 50*150 mm*5 um; mobile phase: [n-Heptane-IPA (0.1% NH₃·H₂O)]; B %: 20%-60%, 10 min) to give two impure product. N-[2-[4-[[[4-[1-(2,6-dioxo-3-piperidyl)-3-methyl-2-oxo-benzimidazol-4-yl]cyclohexyl]-methyl-amino]methyl]cyclohexyl]-6-methoxy-indazol-5-yl]-6-(trifluoromethyl)pyridine-2-carboxamide (0.06 g, 6% yield) was isolated as a white solid. This product was re-purified by prep-HPLC (column: 3_Phenomenex Luna C18 75*30 mm*3 um; mobile phase: [water (0.225% FA)-ACN]; B %: 24%-44%, 8 min) to give the title compound (21.0 mg, 42% yield) as white solid. ¹H NMR (400 MHz, DMSO-d₆) δ 11.09 (s, 1H), 10.50 (s, 1H), 8.68 (s, 1H), 8.45 (d, J=7.6 Hz, 1H), 8.40 (d, J=7.6 Hz, 1H), 8.33 (s, 1H), 8.23-8.20 (m, 1H), 7.15 (s, 1H), 7.03-6.97 (m, 3H), 5.37 (dd, J=5.2, 12.8 Hz, 1H), 4.38-4.35 (m, 1H), 3.98 (s, 3H), 3.59 (s, 3H), 3.24-3.20 (m, 2H), 2.95-2.83 (m, 1H), 2.77-2.60 (m, 2H), 2.33-2.30 (m, 2H), 2.27 (s, 3H), 2.15 (d, J=10.8 Hz, 2H), 2.04-1.87 (m, 9H), 1.70-1.40 (m, 5H), 1.18-1.03 (m, 2H); LC-MS (ESI⁺) m/z 801.3 (M+H)⁺. N-[2-[4-[[[4-[1-(2,6-dioxo-3-piperidyl)-3-methyl-2-oxo-benzimidazol-4-yl]cyclohexyl]amino]methyl]cyclohexyl]-6-methoxy-indazol-5-yl]-6-(trifluoromethyl)pyridine-2-carboxamide (0.06 g, 7% yield) was isolated as white solid. This product was re-purified by prep-HPLC (column: 3_Phenomenex Luna C18 75*30 mm*3 um; mobile phase: [water (0.225% FA)-ACN]; B %: 26%-46%, 8 min) to give the title compound (41.0 mg, 68% yield) as white solid. ¹H NMR (400 MHz, DMSO-d₆) δ 11.08 (s, 1H), 10.50 (s, 1H), 8.68 (s, 1H), 8.45 (d, J=7.6 Hz, 1H), 8.40 (d, J=7.6 Hz, 1H), 8.32 (s, 1H), 8.23-8.20 (m, 1H), 7.16 (s, 1H), 7.03-6.93 (m, 3H), 5.35 (dd, J=5.2, 12.8 Hz, 1H), 4.41-4.35 (m, 1H), 3.97 (s, 3H), 3.59 (s, 3H), 3.24-3.20 (m, 2H), 2.89-2.86 (m, 1H), 2.72-2.67 (m, 1H), 2.65-2.62 (m, 1H) 2.26-2.23 (m, 2H), 2.20 (s, 3H), 2.16-2.13 (m, 2H), 2.04-1.94 (m, 5H), 1.92-1.89 (m, 4H), 1.60-1.55 (m, 5H), 1.16-1.10 (m, 2H); LC-MS (ESI⁺) m/z 801.3 (M+H)⁺. The absolute stereochemistry of the trans and cis-isomers was randomly assigned.

### Example 7: Synthesis of 6-(1,1-Difluoroethyl)-N-[2-[4-[[6-[2-[[2-(2,6-dioxo-3-piperidyl)-1,3-dioxo-isoindolin-4-yl]amino]ethyl]-2-azaspiro[3.3]heptan-2-yl]methyl]cyclohexyl]-6-methoxy-indazol-5-yl]pyridine-2-carboxamide (I-11)

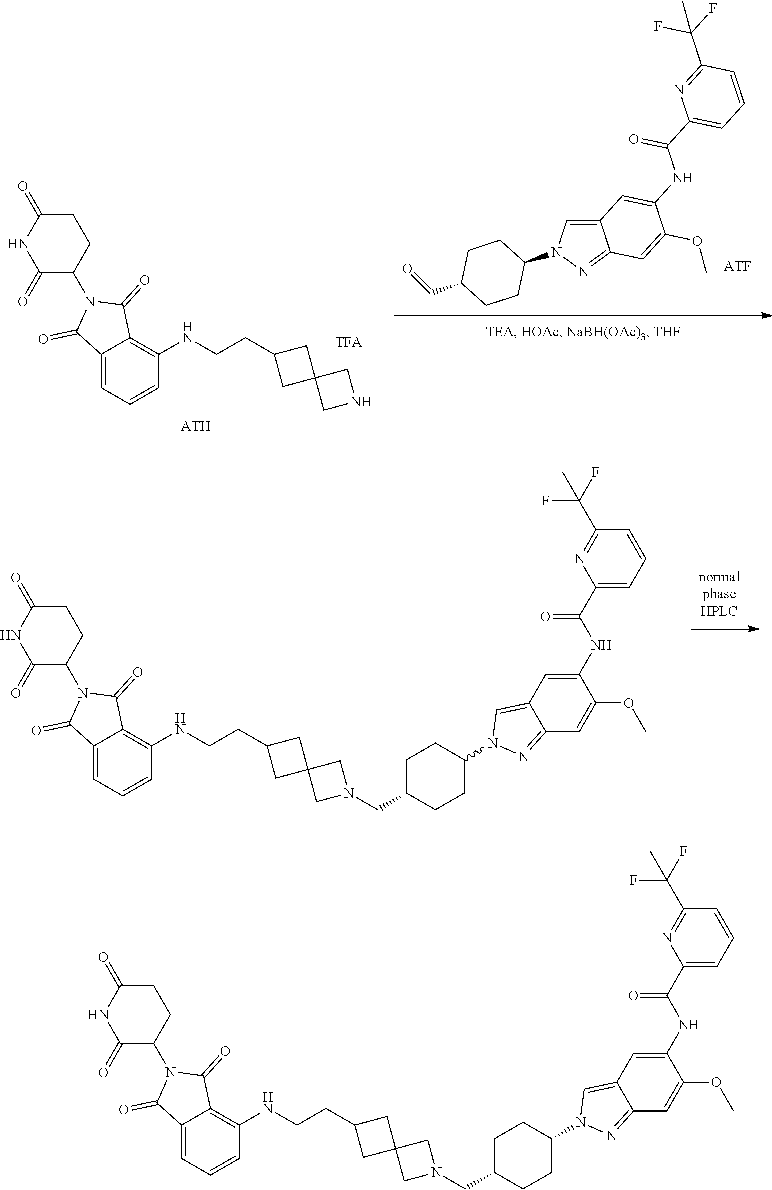

### Step 1—6-(1,1-Difluoroethyl)-N-[2-[4-[[6-[2-[[2-(2,6-dioxo-3-piperidyl)-1,3-dioxo-isoindolin-4-yl]amino]ethyl]-2-azaspiro[3.3]heptan-2-yl]methyl]cyclohexyl]-6-methoxy-indazol-5-yl]pyridine-2-carboxamide

To a mixture of 4-[2-(2-azaspiro[3.3]heptan-6-yl)ethylamino]-2-(2,6-dioxo-3-piperidyl)isoindoline-1,3-dione (50 mg, 97.9 umol, TFA, Intermediate ATH) in THF (1 mL) was added TEA (19.8 mg, 195 umol) and the mixture was stirred at 25° C. for 6 mins. Then HOAc (11.7 mg, 195 umol) and 6-(1,1-difluoroethyl)-N-[2-(4-formylcyclohexyl)-6-methoxy-indazol-5-yl]pyridine-2-carboxamide (43.3 mg, 97.9 umol, Intermediate ATF) and NaBH(OAc)₃ (41.5 mg, 195 umol) was added and the mixture was stirred 50° C. for 16 hrs. On completion, the reaction mixture was quenched with water (1 mL) and extracted with DCM (3×5 mL). The combined organic lays was dried over Na₂SO₄, filtered and concentrated in vacuo to give the title compound (65.0 mg, crude) as yellow oil. The crude product contained some of cis-isomer. LC-MS (ESI⁺) m/z 823.5 (M+H)⁺.

### Step 2—6-(1,1-Difluoroethyl)-N-[2-[4-[[6-[2-[[2-(2,6-dioxo-3-piperidyl)-1,3-dioxo-isoindolin-4-yl]amino]ethyl]-2-azaspiro[3.3]heptan-2-yl]methyl]cyclohexyl]-6-methoxy-indazol-5-yl]pyridine-2-carboxamide

6-(1,1-difluoroethyl)-N-[2-[4-[[6-[2-[[2-(2,6-dioxo-3-piperidyl)-1,3-dioxo-isoindolin-4-yl]amino]ethyl]-2-azaspiro[3.3]heptan-2-yl]methyl]cyclohexyl]-6-methoxy-indazol-5-yl]pyridine-2-carboxamide (189 mg, 230 umol, containing some of cis-isomer) was purified by normal prep-HPLC (column: Waters Vinridis Silica 2-EP OBD 50*150 mm*5 um; mobile phase: [n-Heptane-IPA (0.1% TEA)]; B %: 15%-55%, 10 min) twice and then purified by Prep-HPLC (column: Unisil 3-100 C18 Ultra 150*50 mm*3 um; mobile phase: [water(0.225% FA)-ACN]; B %: 30%-50%, 10 min) to give the title compound (10.4 mg, 15% yield, FA) as yellow solid. ¹H NMR (400 MHz, DMSO-d₆) δ 11.09 (s, 1H), 10.66 (s, 1H), 8.66 (s, 1H), 8.34-8.27 (m, 3H), 8.03-7.99 (m, 1H), 7.62-7.54 (m, 1H), 7.18-7.14 (m, 1H), 7.06 (d, J=8.8 Hz, 1H), 7.02 (d, J=7.2 Hz, 1H), 6.47 (t, J=5.6 Hz, 1H), 5.08-5.02 (m, 1H), 4.48-4.36 (m, 1H), 3.99 (s, 3H), 3.24-3.20 (m, 2H), 3.17 (s, 2H), 3.07 (s, 2H), 2.94-2.83 (m, 1H), 2.63-2.53 (m, 2H), 2.42-2.35 (m, 2H), 2.26-2.06 (m, 8H), 2.06-1.98 (m, 1H), 1.90-1.80 (m, 2H), 1.80-1.72 (m, 2H), 1.68-1.61 (m, 2H), 1.58-1.55 (m, 4H); LC-MS (ESI⁺) m/z 823.5 (M+H)⁺.

### Example 8: Synthesis of N-[2-[2-[[1-[1-(2,6-dioxo-3-piperidyl)-3-methyl-2-oxo-benzimidazol-4-yl]-4-piperidyl]-methyl-amino]ethyl]-6-fluoro-indazol-5-yl]-6-(trifluoromethyl)pyridine-2-carboxamide (1-19)

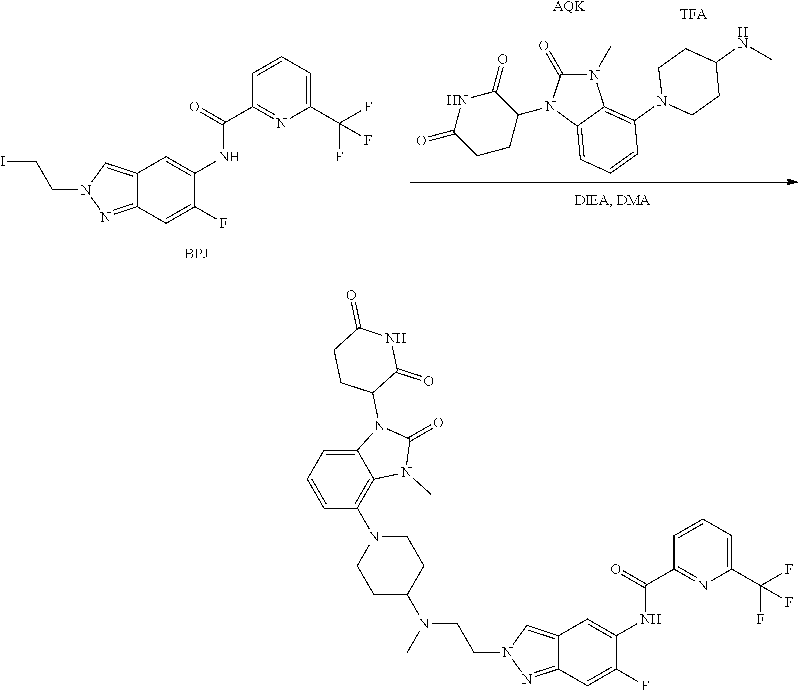

To a solution of 3-[3-methyl-4-[4-(methylamino)-1-piperidyl]-2-oxo-benzimidazol-1-yl]piperidine-2,6-dione (76.1 mg, 156 umol, TFA, Intermediate AQK) and N-[6-fluoro-2-(2-iodoethyl)indazol-5-yl]-6-(trifluoromethyl) pyridine-2-carboxamide (75.0 mg, 156 umol, Intermediate BPJ) in DMA (2.00 mL) was added DIEA (121 mg, 941 umol, 164 uL) at 25° C., and the mixture was stirred at 60° C. for 16 hr. On completion, the reaction mixture was quenched by addition water (0.25 mL), and then concentrated in vacuo to give a residue. The residue was purified by reversed phase (0.10% FA condition) to give the crude product which was re-purified by prep-HPLC (column: Phenomenex luna C18 150*25 mm*10 um; mobile phase: [water (0.225% FA)-ACN]; B %: 17%-47%, 10 min) to give the title compound (4.27 mg, 4% yield, FA) as a white solid. ¹H NMR (400 MHz, DMSO-d₆) δ 11.08 (d, J=3.6 Hz, 1H), 10.23 (d, J=2.0 Hz, 1H), 8.49 (s, 1H), 8.47-8.37 (m, 3H), 8.22 (dd, J=1.2, 7.6 Hz, 1H), 7.55 (d, J=12.0 Hz, 1H), 7.00-6.91 (m, 1H), 6.91-6.80 (m, 2H), 5.39-5.27 (m, 1H), 4.49 (t, J=5.6 Hz, 2H), 3.60 (s, 3H), 3.13-3.05 (m, 2H), 2.99 (t, J=6.4 Hz, 2H), 2.92-2.82 (m, 1H), 2.73-2.64 (m, 3H), 2.63-2.59 (m, 1H), 2.46-2.42 (m, 1H), 2.31 (s, 3H), 2.02-1.94 (m, 1H), 1.74-1.65 (m, 2H), 1.64-1.47 (m, 2H); LC-MS (ESI⁺) m/z 722.4 (M+H)⁺.

### Example 9: Synthesis of N-[2-[2-[[1-[1-(2,6-dioxo-3-piperidyl)-3-methyl-2-oxo-benzimidazol-4-yl]-4-piperidyl]-methyl-amino]ethyl]-6-methoxy-indazol-5-yl]-6-(trifluoromethyl)pyridine-2-carboxamide (I-20)

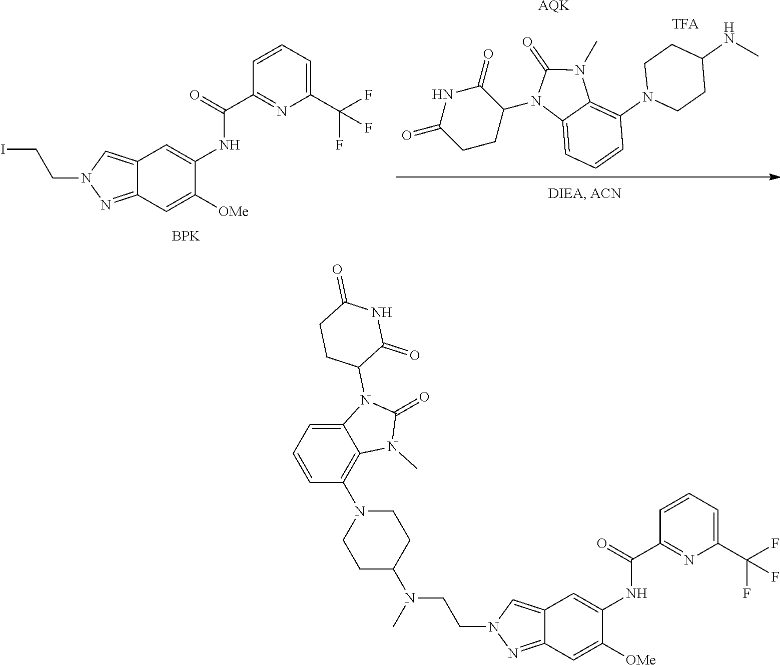

To a solution of N-[2-(2-iodoethyl)-6-methoxy-indazol-5-yl]-6-(trifluoromethyl)pyridine-2-carboxamide (110 mg, 224 umol, Intermediate BPK) and 3-[3-methyl-4-[4-(methylamino)-1-piperidyl]-2-oxo-benzimidazol-1-yl]piperidine-2,6-dione (108 mg, 224 umol, TFA salt, Intermediate AQK) in ACN (4 mL) was added DIEA (87.0 mg, 673 umol). The reaction mixture was stirred at 80° C. for 16 hrs. On completion, the reaction mixture was acidified with FA until the pH=6˜7, and the solution was concentrated in vacuo. The residue was purified by Prep-HPLC (column: Phenomenex Synergi C18 150*25 mm*10 um; mobile phase: [water (0.225% FA)-ACN]) to give the title compound (50.0 mg, 28% yield, FA salt) as yellow solid. ¹H NMR (400 MHz, DMSO-d₆) δ 11.07 (s, 1H), 10.50 (s, 1H), 8.69 (s, 1H), 8.49-8.45 (m, 1H), 8.43-8.38 (m, 1H), 8.33 (s, 1H), 8.22 (dd, J=0.8, 8.0 Hz, 1H), 7.16 (s, 1H), 7.00-6.81 (m, 3H), 5.33 (dd, J=5.2, 12.8 Hz, 1H), 4.43 (t, J=6.0 Hz, 2H), 3.98 (s, 3H), 3.60 (s, 3H), 3.14-3.05 (m, 2H), 2.98 (t, J=6.0 Hz, 2H), 2.93-2.82 (m, 1H), 2.70-2.58 (m, 4H), 2.46-2.44 (m, 1H), 2.30 (s, 3H), 2.04-1.92 (m, 1H), 1.77-1.68 (m, 2H), 1.65-1.51 (m, 2H); LC-MS (ESI⁺) m/z 734.5 (M+H)⁺.

### Example 10: Synthesis of N-[2-[2-[[1-[1-(2,6-dioxo-3-piperidyl)-3-methyl-2-oxo-benzimidazol-4-yl]-4-piperidyl]-methyl-amino]ethyl]-6-methoxy-indazol-5-yl]pyrazolo[1,5-a]pyrimidine-3-carboxamide (I-21)

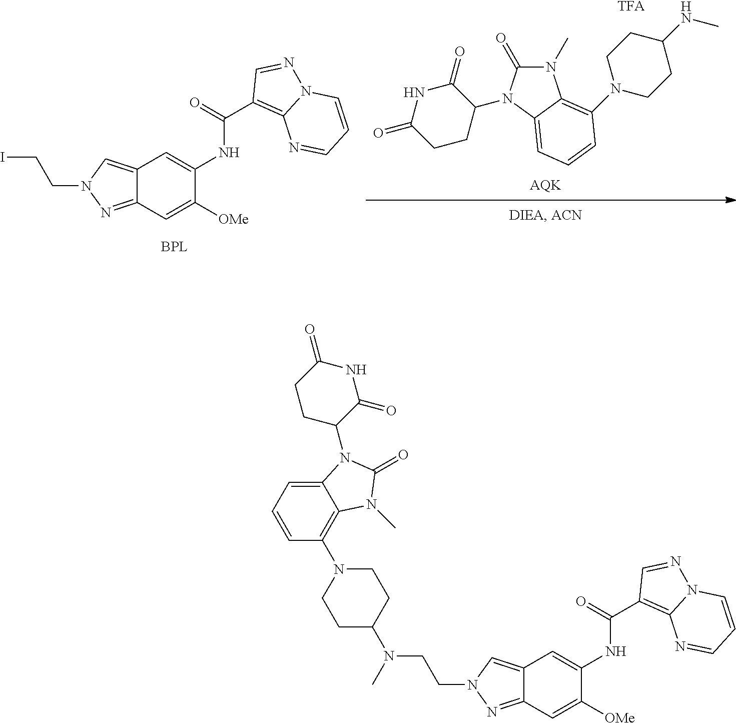

To a solution of 3-[3-methyl-4-[4-(methylamino)-1-piperidyl]-2-oxo-benzimidazol-1-yl]piperidine-2,6-dione (178 mg, 367 umol, TFA salt, Intermediate AQK) and DIEA (142 mg, 1.10 mmol) in ACN (5 mL) was added N-[2-(2-iodoethyl)-6-methoxy-indazol-5-yl]pyrazolo[1,5-a]pyrimidine-3-carboxamide (170 mg, 367 umol, Intermediate BPL). The reaction mixture was stirred at 80° C. for 16 hrs. On completion, the reaction mixture was acidified with FA until the pH=6˜7, and the mixture was concentrated in vacuo. The residue was purified by Prep-HPLC (column: Phenomenex Synergi C18 150*25 mm*10 um; mobile phase: [water (0.225% FA)-ACN]) to give the title compound (33.4 mg, 11% yield, FA salt) as yellow solid. ¹H NMR (400 MHz, DMSO-d₆) δ 11.07 (s, 1H), 10.53 (s, 1H), 9.38 (dd, J=1.6, 7.2 Hz, 1H), 8.96 (dd, J=1.6, 4.0 Hz, 1H), 8.77-8.64 (m, 2H), 8.27 (s, 1H), 7.35 (dd, J=4.0, 7.2 Hz, 1H), 7.11 (s, 1H), 7.01-6.77 (m, 3H), 5.33 (dd, J=4.8, 12.4 Hz, 1H), 4.42 (t, J=6.0 Hz, 2H), 4.03 (s, 3H), 3.60 (s, 3H), 3.14-3.06 (m, 2H), 2.98 (t, J=6.4 Hz, 2H), 2.91-2.82 (m, 1H), 2.75-2.60 (m, 4H), 2.60-2.53 (m, 1H), 2.31 (s, 3H), 2.05-1.90 (m, 1H), 1.79-1.68 (m, 2H), 1.67-1.52 (m, 2H); LC-MS (ESI⁺) m/z 706.5 (M+H)⁺.

### Example 11: Synthesis of N-[2-[2-[[1-[1-(2,6-dioxo-3-piperidyl)-3-methyl-2-oxo-benzimidazol-4-yl]-4-piperidyl]-methyl-amino]ethyl]-6-fluoro-indazol-5-yl]pyrazolo[1,5-a]pyrimidine-3-carboxamide (I-22)

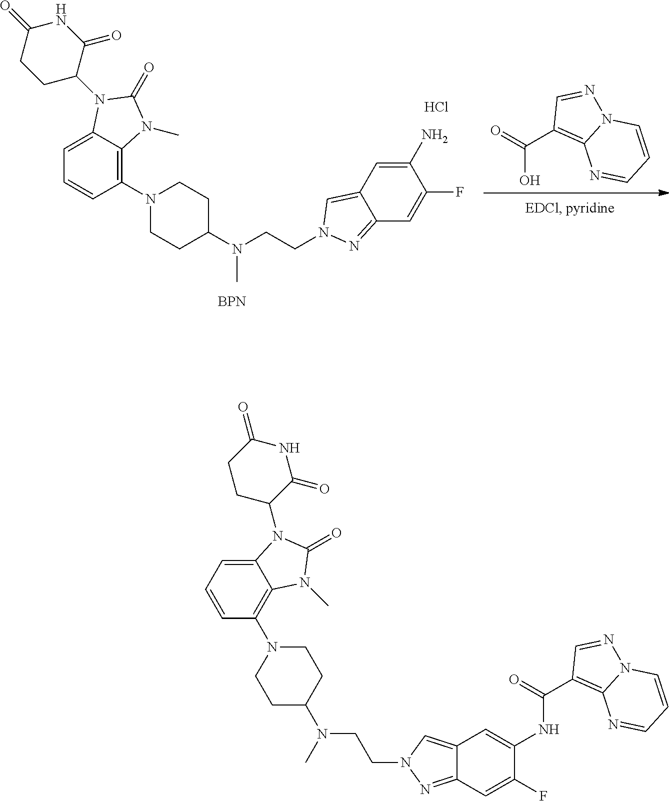

To a solution of 3-[4-[4-[2-(5-amino-6-fluoro-indazol-2-yl)ethyl-methyl-amino]-1-piperidyl]-3-methyl-2-oxo-benzimidazol-1-yl]piperidine-2,6-dione (10.0 mg, 17.1 umol, HCl, Intermediate BPN) and pyrazolo[1,5-a]pyrimidine-3-carboxylic acid (3.07 mg, 18.8 umol, CAS #25940-35-6) in pyridine (0.50 mL) was added EDCI (3.93 mg, 20.5 umol). The mixture was stirred at 25° C. for 1 hr. On completion, the reaction mixture was quenched with water (0.05 mL) and then concentrated in vacuo to give a residue. The residue was purified by prep-HPLC (column: Phenomenex luna C18 150*25 mm*10 um; mobile phase: [water (0.225% FA)-ACN]; B %: 10%-40%, 10 min) to give the title compound (4.46 mg, 35% yield, FA) as a white solid. ¹H NMR (400 MHz, DMSO-d₆) δ¹H NMR (400 MHz, DMSO-d₆) δ=11.08 (s, 1H), 10.18 (d, J=3.2 Hz, 1H), 9.40 (dd, J=1.6, 7.2 Hz, 1H), 8.93 (dd, J=1.6, 4.4 Hz, 1H), 8.75 (s, 1H), 8.67 (d, J=7.6 Hz, 1H), 8.45 (s, 1H), 7.54 (d, J=12.0 Hz, 1H), 7.36 (dd, J=4.4, 7.0 Hz, 1H), 7.00-6.79 (m, 3H), 5.32 (dd, J=4.8, 12.4 Hz, 1H), 4.47 (t, J=6.4 Hz, 2H), 3.59 (s, 3H), 3.13-3.05 (m, 2H), 2.98 (t, J=6.4 Hz, 2H), 2.92-2.83 (m, 1H), 2.72-2.67 (m, 1H), 2.66-2.56 (m, 3H), 2.46-2.41 (m, 1H), 2.30 (s, 3H), 2.01-1.93 (m, 1H), 1.73-1.66 (m, 2H), 1.57 (dd, J=1.2, 10.8 Hz, 2H); LC-MS (ESI⁺) m/z 694.2 (M+H)⁺.

### Examples 12: Syntheses of N-[2-[3-[[[1-[1-(2,6-dioxo-3-piperidyl)-3-methyl-2-oxo-benzimidazol-4-yl]-4-piperidyl]-methyl-amino]methyl]cyclobutyl]-6-methoxy-indazol-5-yl]-6-(trifluoromethyl)pyridine-2-carboxamide (I-31) and N-[2-[3-[[[1-[1-(2,6-dioxo-3-piperidyl)-3-methyl-2-oxo-benzimidazol-4-yl]-4-piperidyl]-methyl-amino]methyl]cyclobutyl]-6-methoxy-indazol-5-yl]-6-(trifluoromethyl)pyridine-2-carboxamide (I-32)

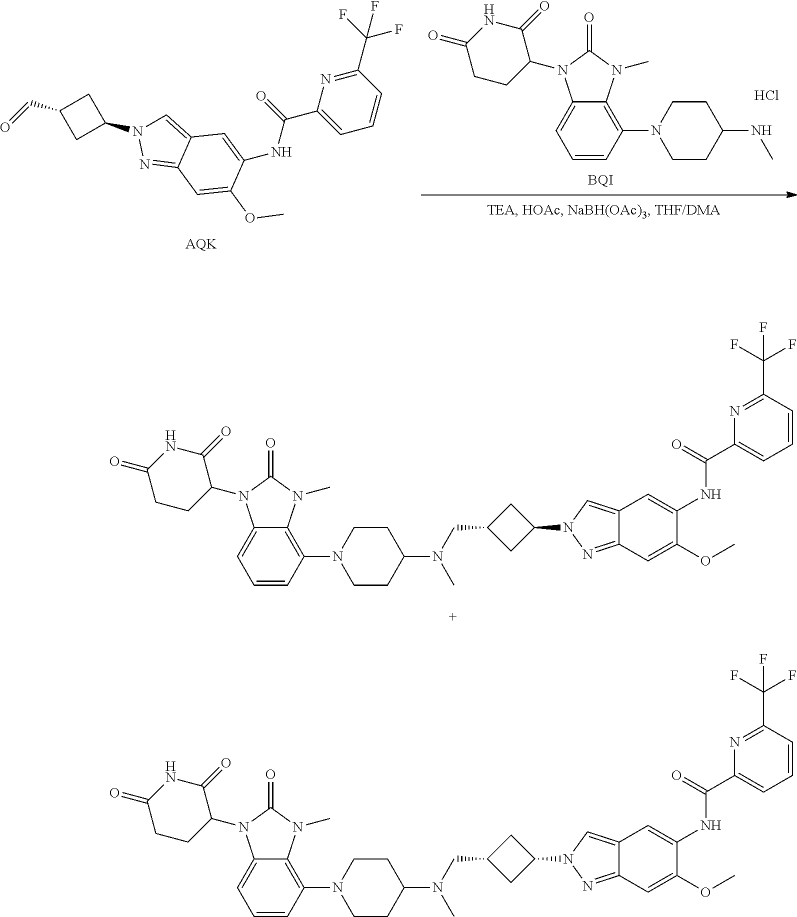

To a solution of 3-[3-methyl-4-[4-(methylamino)-1-piperidyl]-2-oxo-benzimidazol-1-yl]piperidine-2,6-dione (53.6 mg, 131 umol, HCl salt, Intermediate AQK) in THF (0.5 mL) and DMA (1 mL) was added TEA (13.3 mg, 131 umol), then the mixture stirred at 25° C. for 10 min. Next, HOAc (7.89 mg, 131 umol) and N-[2-(3-formylcyclobutyl)-6-methoxy-indazol-5-yl]-6-(trifluoromethyl)pyridine-2-carboxamide (55.0 mg, 131 umol, Intermediate BQI) was added to the mixture and was stirred at 25° C. for 20 minutes. Next, NaBH(OAc)₃ (55.7 mg, 262 umol) was added to the mixture at 0° C. and the reaction mixture was stirred at −20-0° C. for 2 hrs. On completion, H₂O (0.5 mL) was added to the reaction mixture, then the mixture was concentrated in vacuo. The residue was purified by prep-HPLC (column: Phenomenex Gemini-NX C18 75*30 mm*3 um; mobile phase: [water(0.225% FA)-ACN]; B %: 20%-50%, 7 min) and prep-HPLC (column: Welch Ultimate XB-SiOH 250*50*10 um; mobile phase: [Hexane-IPA]; B %: 50%-90%, 15 min) to give N-[2-[3-[[[1-[1-(2,6-dioxo-3-piperidyl)-3-methyl-2-oxo-benzimidazol-4-yl]-4-piperidyl]-methyl-amino]methyl]cyclobutyl]-6-methoxy-indazol-5-yl]-6-(trifluoromethyl)pyridine-2-carboxamide (4.51 mg, 11% yield) (¹HNMR (400 MHz, DMSO-d₆) δ 11.09 (s, 1H), 10.51 (s, 1H), 8.69 (s, 1H), 8.49-8.36 (m, 3H), 8.22 (d, J=7.6 Hz, 1H), 7.20 (s, 1H), 7.03-6.85 (m, 3H), 5.35 (dd, J=5.2, 13.2 Hz, 1H), 5.25-5.17 (m, 1H), 3.99 (s, 3H), 3.66 (s, 3H), 3.18 (d, J=11.2 Hz, 4H), 2.93-2.85 (m, 2H), 2.77-2.68 (m, 6H), 2.60 (s, 3H), 2.30-2.21 (m, 3H), 2.02-1.98 (m, 1H), 1.90-1.81 (m, 2H), 1.78-1.69 (m, 2H); LC-MS (ESI+) m/z 774.5 (M+H)⁺) and N-[2-[3-[[[1-[1-(2,6-dioxo-3-piperidyl)-3-methyl-2-oxo-benzimidazol-4-yl]-4-piperidyl]-methyl-amino]methyl]cyclobutyl]-6-methoxy-indazol-5-yl]-6-(trifluoromethyl)pyridine-2-carboxamide (23.0 mg, 57% yield) (¹H NMR (400 MHz, DMSO-d₆) δ 11.08 (s, 1H), 10.51 (s, 1H), 8.68 (s, 1H), 8.49-8.36 (m, 3H), 8.22 (d, J=7.6 Hz, 1H), 7.19 (s, 1H), 7.00-6.85 (m, 3H), 5.39-5.30 (m, 1H), 5.02-4.90 (m, 1H), 3.99 (s, 3H), 3.65 (s, 3H), 3.17 (d, J=9.6 Hz, 4H), 2.96-2.84 (m, 2H), 2.78-2.68 (m, 4H), 2.65-2.57 (m, 5H), 2.30-2.24 (m, 3H), 2.03-1.96 (m, 1H), 1.90-1.79 (m, 2H), 1.75-1.61 (m, 2H); LC-MS (ESI+) m/z 774.5 (M+H)⁺) as white solids. The structures of these two isomers were confirmed by 2D NMR.

### Examples 13: Syntheses of N-[2-[3-[[3-[1-(2,6-dioxo-3-piperidyl)-3-methyl-2-oxo-benzimidazol-4-yl]azetidin-1-yl]methyl]cyclo butyl]-6-methoxy-indazol-5-yl]-6-(trifluoromethyl)pyridine-2-carboxamide (I-46) and N-[2-[3-[[3-[1-(2,6-dioxo-3-piperidyl)-3-methyl-2-oxo-benzimidazol-4-yl]azetidin-1-yl]methyl]cyclobutyl]-6-methoxy-indazol-5-yl]-6-(trifluoromethyl)pyridine-2-carboxamide (I-50)

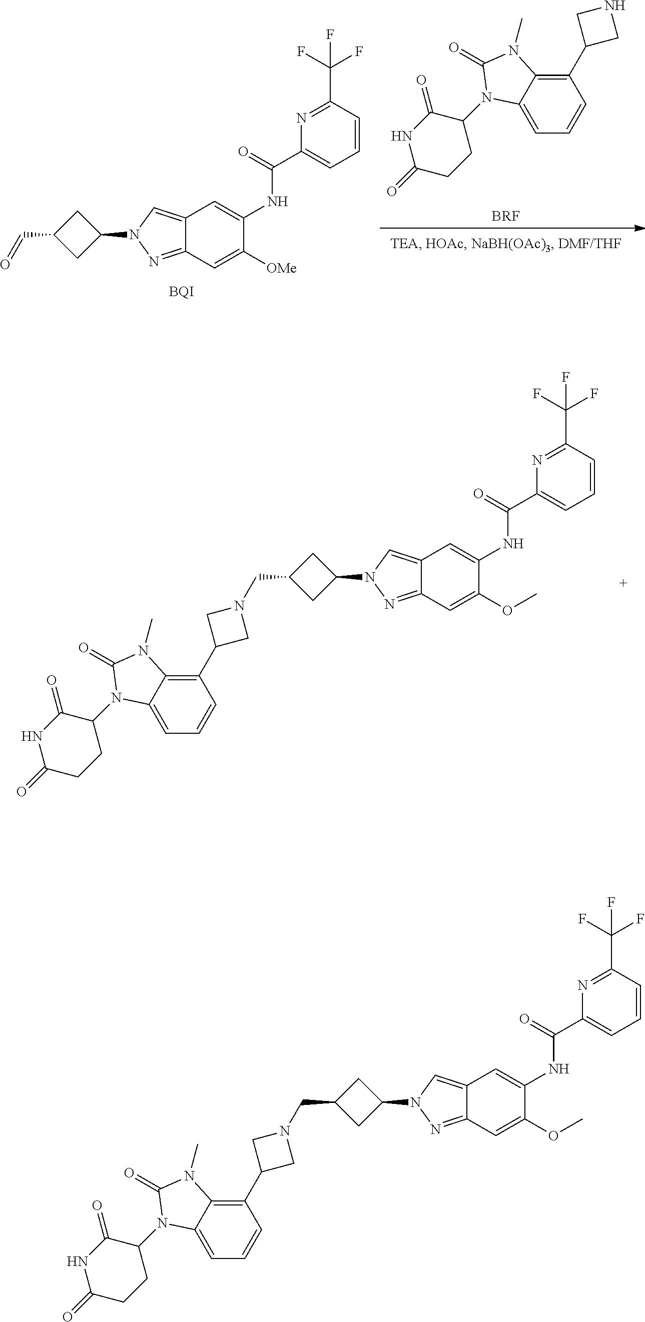

To a solution of 3-[4-(azetidin-3-yl)-3-methyl-2-oxo-benzimidazol-1-yl]piperidine-2,6-dione (50.0 mg, 116 umol, TFA, Intermediate BRF) in THF (1 mL) and DMF (1 mL) was added TEA (11.8 mg, 116 umol), and the reaction mixture was stirred at −10° C. for 10 mins. Then N-[2-(3-formylcyclobutyl)-6-methoxy-indazol-5-yl]-6-(trifluoromethyl)pyridine-2-carboxamide (45.0 mg, 107 umol, Intermediate BQI) and HOAc (14.0 mg, 233 umol) was added to the mixture at −10° C. and stirred for 20 mins. Next, NaBH(OAc)₃ (29.7 mg, 140 umol) was added to the mixture and the mixture was stirred at −10° C. for 1 hr. On completion, the reaction mixture was quenched with water (0.05 mL) and concentrated in vacuo to give a residue. The residue was purified by prep-HPLC(column: Phenomenex luna C18 150*25 mm*10 um; mobile phase: [water(0.225% FA)-ACN]; B %: 16%-46%, 11.5 min) and purified by prep-HPLC(column: Welch Ultimate XB SiO2 10u 100*30 mm; mobile phase: [IPA-ACN]; B %: 85%-95%, 10 min). Then the residue was further purified by prep-HPLC(column: Phenomenex luna C18 150*25 mm*10 um; mobile phase: [water(0.225% FA)-ACN]; B %: 20%-50%, 11 min) to give the title compound N-[2-[3-[[3-[1-(2,6-dioxo-3-piperidyl)-3-methyl-2-oxo-benzimidazol-4-yl]azetidin-1-yl]methyl]cycl obutyl]-6-methoxy-indazol-5-yl]-6-(trifluoromethyl)pyridine-2-carboxamide (13.98 mg, 15% yield, FA) (¹H NMR (400 MHz, DMSO-d₆) δ 11.09 (s, 1H), 10.51 (s, 1H), 8.69 (s, 1H), 8.49-8.44 (m, 1H), 8.41 (t, J=8.0 Hz, 1H), 8.36 (s, 1H), 8.22 (dd, J=0.9, 7.6 Hz, 1H), 7.23-7.17 (m, 2H), 7.10-7.04 (m, 1H), 7.04-6.99 (m, 1H), 5.37 (dd, J=5.2, 12.4 Hz, 1H), 5.24-5.11 (m, 1H), 4.32-4.19 (m, 1H), 3.99 (s, 3H), 3.69 (t, J=6.8 Hz, 2H), 3.53 (s, 3H), 3.27 (s, 4H), 2.93-2.84 (m, 1H), 2.77-2.67 (m, 3H), 2.66-2.57 (m, 2H), 2.35 (d, J=3.2 Hz, 1H), 2.31 (s, 1H), 2.04-1.95 (m, 1H); LC-MS (ESI⁺) m/z 717.2 (M+H)⁺) and N-[2-[3-[[3-[1-(2,6-dioxo-3-piperidyl)-3-methyl-2-oxo-benzimidazol-4-yl]azetidin-1-yl]methyl]cyclobutyl]-6-methoxy-indazol-5-yl]-6-(trifluoromethyl)pyridine-2-carboxamide (5.77 mg, 6.48% yield, FA) (¹H NMR (400 MHz, DMSO-d₆) δ 11.09 (s, 1H), 10.50 (s, 1H), 8.68 (s, 1H), 8.49-8.44 (m, 1H), 8.41 (t, J=7.6 Hz, 1H), 8.36 (s, 1H), 8.22 (d, J=7.6 Hz, 1H), 7.24-7.16 (m, 2H), 7.11-6.96 (m, 2H), 5.37 (dd, J=5.6, 12.8 Hz, 1H), 5.03-4.88 (m, 1H), 4.31-4.17 (m, 1H), 3.99 (s, 3H), 3.67 (t, J=7.2 Hz, 2H), 3.52 (s, 3H), 3.25 (s, 4H), 2.94-2.82 (m, 1H), 2.65-2.56 (m, 5H), 2.33 (s, 1H), 2.24-2.18 (m, 1H), 1.99 (dd, J=4.8, 6.8 Hz, 1H). LC-MS (ESI⁺) m/z 717.2 (M+H)⁺) as white solids.

### Examples 14: Synthesis of N-[2-[4-[[3-[1-(2,6-dioxo-3-piperidyl)-3-methyl-2-oxo-benzimidazol-4-yl]-3,6-diazabicyclo [3.1.1]heptan-6-yl]methyl]cyclohexyl]indazol-5-yl]-6-(trifluoromethyl)pyridine-2-carboxamide (I-157) & N-[2-[4-[[3-[1-(2,6-dioxo-3-piperidyl)-3-methyl-2-oxo-benzimidazol-4-yl]-3,6-diazabicyclo[3.1.1]heptan-6-yl]methyl]cyclohexyl]indazol-5-yl]-6-(trifluoromethyl)pyridine-2-carboxamide (I-156)

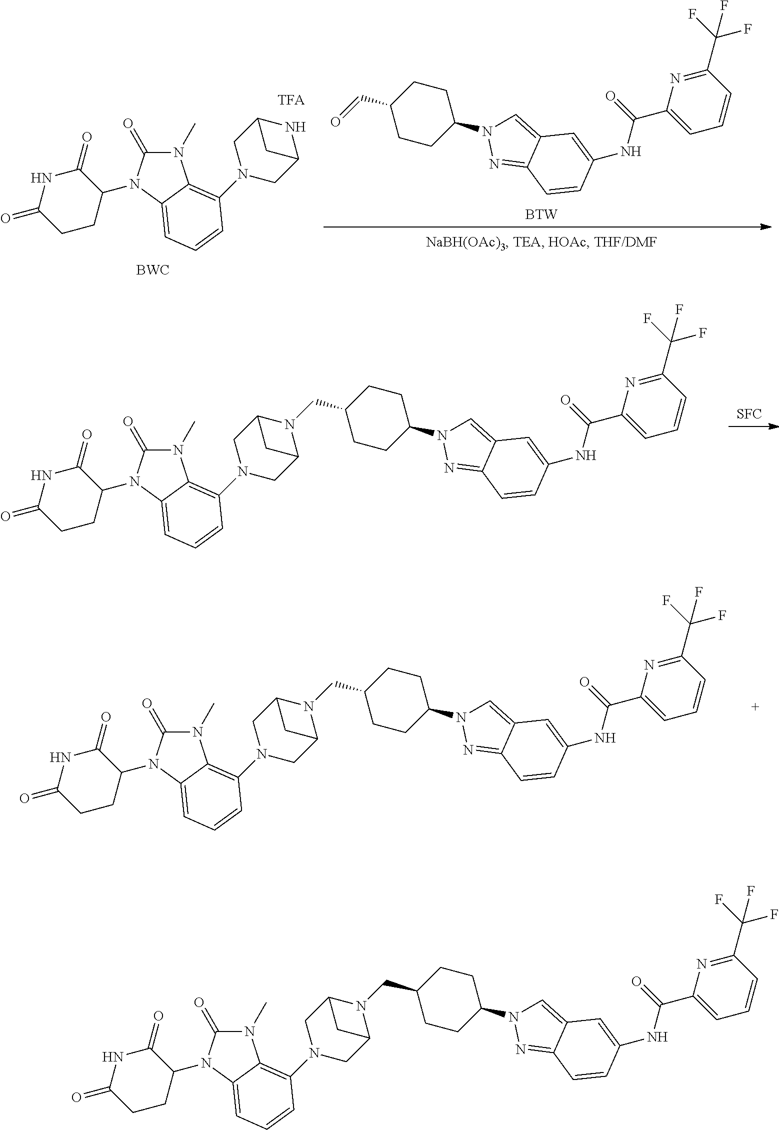

### Step 1—N-[2-[4-[[3-[1-(2,6-dioxo-3-piperidyl)-3-methyl-2-oxo-benzimidazol-4-yl]-3,6-diazabicyclo [3.1.1]heptan-6-yl]methyl]cyclohexyl]indazol-5-yl]-6-(trifluoromethyl)pyridine-2-carboxamide

To a mixture of 3-[4-(3,6-diazabicyclo[3.1.1]heptan-3-yl)-3-methyl-2-oxo-benzimidazol-1-yl]piperidine-2,6-dione (80.0 mg, 170 umol, TFA, Intermediate BWC) in THF (1 mL) and DMF (0.5 mL) was added TEA (17.2 mg, 170 umol, 23.7 uL), N-[2-(4-formlylcyclohexyl)indazol-5-yl]-6-(trifluoromethyl)pyridine-2-carboxamide (70.9 mg, 170 umol, Intermediate BTW) and HOAc (10.2 mg, 170 umol, 9.75 uL) at −10° C. The reaction mixture was stirred at −10° C. for 0.5 hour, then NaBH(OAc)₃ (54.1 mg, 255 umol) was added to the mixture. The reaction mixture was then stirred at −10° C. for 3.5 hours. On completion, the reaction mixture was concentrated in vacuo. The residue was purified by prep-HPLC (column: Phenomenex luna C18 150*25 mm*10 um; mobile phase: [water(0.225% FA)-ACN]; B %: 18%-48%, 10 min) to give the title compound (31.0 mg, 22% yield, FA) as white solid. ¹H NMR (400 MHz, DMSO-d₆) δ 11.11 (s, 1H), 10.36 (s, 1H), 8.49-8.28 (m, 4H), 8.20-8.15 (m, 1H), 7.65-7.53 (m, 2H), 7.49-7.29 (m, 1H), 7.12 (t, J=7.6 Hz, 1H), 7.01 (d, J=7.2 Hz, 1H), 5.43-5.34 (m, 1H), 4.54-4.43 (m, 1H), 3.79-3.64 (m, 2H), 3.57 (s, 3H), 3.19 (d, J=12.8 Hz, 2H), 2.94-2.85 (m, 1H), 2.78-2.58 (m, 4H), 2.52 (s, 2H), 2.20 (dd, J=1.6, 9.6 Hz, 2H), 2.06-1.94 (m, 5H), 1.93-1.88 (m, 1H), 1.76-1.76 (m, 1H), 1.79-1.66 (m, 1H), 1.40-1.22 (m, 2H); LC-MS (ESI⁺) m/z 756.4 (M+H)⁺.

### Step 2—N-[2-[4-[[3-[1-(2,6-dioxo-3-piperidyl)-3-methyl-2-oxo-benzimidazol-4-yl]-3,6-diazabicyclo [3.1.1]heptan-6-yl]methyl]cyclohexyl]indazol-5-yl]-6-(trifluoromethyl)pyridine-2-carboxamide & N-[2-[4-[[3-[1-(2,6-dioxo-3-piperidyl)-3-methyl-2-oxo-benzimidazol-4-yl]-3,6-diazabicyclo[3.1.1]heptan-6-yl]methyl]cyclohexyl]indazol-5-yl]-6-(trifluoromethyl)pyridine-2-carboxamide

N-[2-[4-[[3-[1-(2,6-dioxo-3-piperidyl)-3-methyl-2-oxo-benzimidazol-4-yl]-3,6-diazabicyclo[3.1.1]heptan-6-yl]methyl]cyclohexyl]indazol-5-yl]-6-(trifluoromethyl)pyridine-2-carboxamide (31.0 mg, 38.6 umol, FA) was separated by SFC (column: DAICEL CHIRALCEL OD(250 mm*50 mm, 10 um); mobile phase: [Hexane-IPA]; B %: 40%-40%, 11.5; 130 mm) to give the compounds P1 and P2. The compound P1 was repurified by prep-HPLC (column: Phenomenex Synergi Polar-RP 100*25 mm*4 um; mobile phase: [water(0.225% FA)-ACN]; B %: 23%-53%, 8 min) to give N-[2-[4-[[3-[1-(2,6-dioxo-3-piperidyl)-3-methyl-2-oxo-benzimidazol-4-yl]-3,6-diazabicyclo [3.1.1]heptan-6-yl]methyl]cyclohexyl]indazol-5-yl]-6-(trifluoromethyl)pyridine-2-carboxamide (4.64 mg, 27% yield, 100% purity, FA) as white solid (¹H NMR (400 MHz, DMSO-d₆) δ 11.11 (s, 1H), 10.36 (s, 1H), 8.43-8.35 (m, 3H), 8.30 (s, 1H), 8.19-8.14 (m, 1H), 8.14-8.14 (m, 1H), 7.63-7.60 (m, 1H), 7.57-7.53 (m, 1H), 7.42-7.30 (m, 1H), 7.11 (t, J=8.0 Hz, 1H), 6.98 (d, J=7.2 Hz, 1H), 5.38 (dd, J=5.6, 12.8 Hz, 1H), 4.53-4.42 (m, 1H), 3.72-3.62 (m, 2H), 3.57 (s, 3H), 3.48-3.39 (m, 2H), 3.20-3.05 (m, 2H), 2.94-2.85 (m, 1H), 2.77-2.60 (m, 4H), 2.18 (d, J=11.2 Hz, 3H), 2.01 (d, J=13.6 Hz, 4H), 1.97-1.88 (m, 2H), 1.72-1.51 (m, 1H), 1.33-1.23 (m, 2H); LC-MS (ESI⁺) m/z 756.3 (M+H)⁺). ⁺). The compound P2 was repurified by prep-HPLC (column: Phenomenex Synergi Polar-RP 100*25 mm*4 um; mobile phase: [water(0.225% FA)-ACN]; B %: 23%-53%, 8 min) to give N-[2-[4-[[3-[1-(2,6-dioxo-3-piperidyl)-3-methyl-2-oxo-benzimidazol-4-yl]-3,6-diazabicyclo[3.1.1]heptan-6-yl]methyl]cyclohexyl]indazol-5-yl]-6-(trifluoromethyl)pyridine-2-carboxamide (1.00 mg, 5% yield, FA) as white solid (¹H NMR (400 MHz, DMSO-d₆) δ 11.14-11.05 (m, 1H), 10.36 (s, 1H), 8.47 (s, 1H), 8.41-8.38 (m, 1H), 8.35 (d, J=8.0 Hz, 1H), 8.29 (s, 1H), 8.17 (d, J=7.6 Hz, 1H), 7.65-7.61 (m, 1H), 7.57-7.53 (m, 1H), 7.32 (d, J=8.0 Hz, 1H), 7.09 (t, J=8.0 Hz, 1H), 6.95 (d, J=8.0 Hz, 1H), 5.37 (dd, J=5.6, 12.4 Hz, 1H), 4.57-4.50 (m, 1H), 3.60 (d, J=6.0 Hz, 4H), 3.57 (s, 3H), 3.03 (d, J=10.8 Hz, 2H), 2.94-2.84 (m, 2H), 2.75-2.70 (m, 1H), 2.60 (d, J=2.8 Hz, 1H), 2.25-2.18 (m, 2H), 2.02-1.97 (m, 2H), 1.96-1.90 (m, 2H), 1.79-1.69 (m, 6H), 1.28-1.05 (m, 2H); LC-MS (ESI⁺) m/z 756.3 (M+H)). The absolute stereochemistry was arbitrarily assigned.

### Example 15: Synthesis of cyano-N-[2-[4-[[4-[4-(2,4-dioxohexahydropyrimidin-1-yl)-7-isoquinolyl]-1-piperidyl]methyl]cyclohexyl]indazol-5-yl]pyrazolo[1,5-a]pyrimidine-3-carboxamide (I-170)

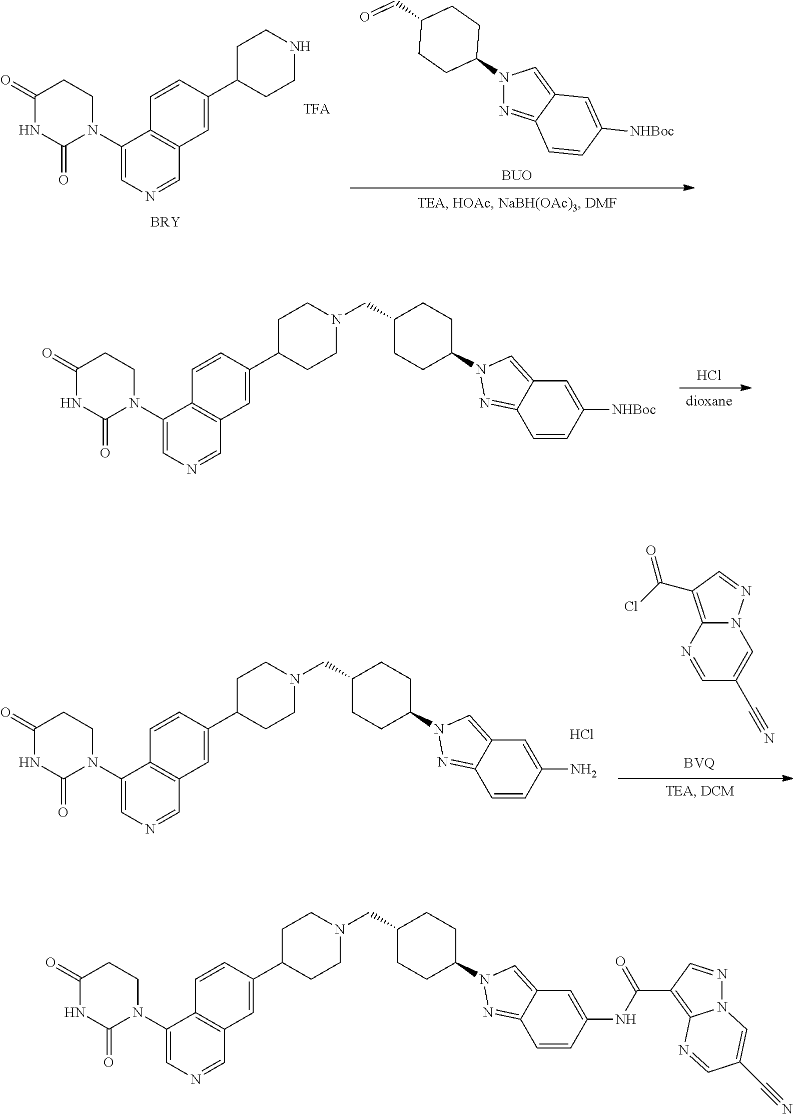

### Step 1—Tert-butyl N-[2-[4-[[4-[4-(2, 4-dioxohexahydropyrimidin-1-yl)-7-isoquinolyl]-1-piperidyl]methyl]cyclohexyl]indazol-5-yl]carbamate

To a mixture of 1-[7-(4-piperidyl)-4-isoquinolyl]hexahydropyrimidine-2,4-dione (206 mg, 469 umol, TFA salt, Intermediate BRY) in DMF (2 mL) was added TEA (47.5 mg, 469 umol) at 25° C. until the pH stabilized at 8. The mixture was stirred at 25° C. for 10 mins, then HOAc (28.2 mg, 469 umol) was added at −15° C. until pH stabilized at 5˜6. Then tert-butyl N-[2-(4-formylcyclohexyl) indazol-5-yl]carbamate (161 mg, 469 umol, Intermediate BUO) was added. The mixture was stirred at −15° C. for 20 mins. Subsequently, NaBH(OAc)₃ (129 mg, 610 umol) was added and the reaction mixture was stirred for another 0.5 hr. On completion, the mixture was quenched with H₂O (0.5 mL). The residue was purified by reverse phase (0.1% FA condition) to give the title compound (210 mg, 68% yield) as a pink solid. LC-MS (ESI+) m/z 652.3 (M+H)⁺.

### Step 2—1-[7-[1-[[4-(5-Aminoindazol-2-yl)cyclohexyl]methyl]-4-piperidyl]-4-isoquinolyl]hexahydropyrimidine-2,4-dione

Tert-butyl N-[2-[4-[[4-[4-(2, 4-dioxohexahydropyrimidin-1-yl)-7-isoquinolyl]-1-piperidyl]methyl]cyclohexyl]indazol-5-yl]carbamate (60.0 mg, 92.0 umol) was dissolved in HCl/dioxane (4 M, 1.15 mL). The mixture was then stirred at 20° C. for 1 hr. On completion, the reaction mixture was concentrated in vacuo to give the title compound (50.0 mg, 98% yield) as a pink solid. LC-MS (ESI+) m/z 552.4 (M+H)⁺.

### Step 3—6-Cyano-N-[2-[4-[[4-[4-(2,4-dioxohexahydropyrimidin-1-yl)-7-isoquinolyl]-1-piperidyl] methyl]cyclohexyl]indazol-5-yl]pyrazolo[1,5-a]pyrimidine-3-carboxamide

To a mixture of 1-[7-[1-[[4-(5-aminoindazol-2-yl)cyclohexyl]methyl]-4-piperidyl]-4-isoquinolyl]hexahydropyrimidine-2,4-dione (50.0 mg, 85.0 umol, HCl salt) in DCM (2 mL) was added TEA (17.2 mg, 170 umol), then 6-cyanopyrazolo[1,5-a]pyrimidine-3-carbonyl chloride (17.5 mg, 85.0 umol, Intermediate BVQ) was added dropwise at 0° C. The mixture was then stirred at 0° C. for 0.5 hr. On completion, the reaction mixture was concentrated in vacuo. The residue was purified by prep-HPLC (column: Waters xbridge 150*25 mm 10 um; mobile phase: [water (10 mM NH4HCO3)-ACN]; B %: 23%-53%, 11.5 min) to give the title compound (2.49 mg, 4.1% yield) as a yellow solid. ¹H NMR (400 MHz, DMSO-d₆) δ10.53 (s, 1H), 10.22 (d, J=2.0 Hz, 1H), 9.82 (s, 1H), 9.26 (s, 1H), 9.16 (d, J=2.0 Hz, 1H), 8.93 (s, 1H), 8.48 (s, 1H), 8.39 (s, 1H), 8.24 (d, J=1.2 Hz, 1H), 8.07 (s, 1H), 7.95-7.90 (m, 1H), 7.95-7.89 (m, 1H), 7.63 (d, J=9.2 Hz, 1H), 7.35 (dd, J=1.6, 9.2 Hz, 1H), 4.52-4.39 (m, 1H), 4.03-3.82 (m, 1H), 3.77-3.68 (m, 1H), 3.06-2.89 (m, 3H), 2.83-2.71 (m, 2H), 2.24-2.15 (m, 4H), 2.06-1.79 (m, 10H), 1.73-1.59 (m, 1H), 1.22-1.12 (m, 2H); LC-MS (ESI+) m/z 722.4 (M+H)⁺.

### Example 16: Synthesis of 6-Cyano-N-[2-[4-[[4-[4-(2,4-dioxohexahydropyrimidin-1-yl)-8-isoquinolyl]-1-piperidyl]methyl]cyclohexyl]indazol-5-yl]pyrazolo[1,5-a]pyrimidine-3-carboxamide (I-204)

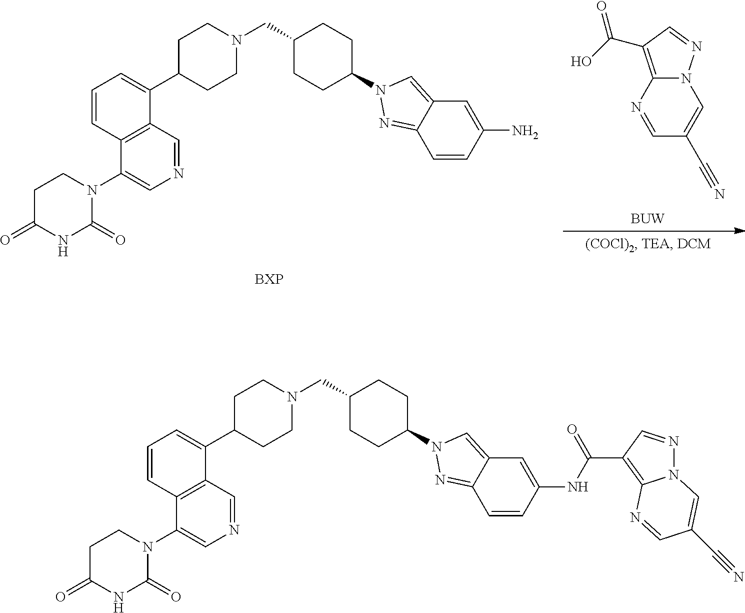 [SMILES: Nc1ccc2nn([C@H]3CC[C@H](CN4CCC(c5cccc6c(N7CCC(=O)NC7=O)cncc56)CC4)CC3)cc2c1 ; N#Cc1cnc2c(C(=O)O)cnn2c1 ; "BUW" ; "(COCl)2, TEA, DCM" ; N#Cc1cnc2c(C(=O)Nc3ccc4nn([C@H]5CC[C@H](CN6CCC(c7cccc8c(N9CCC(=O)NC9=O)cncc78)CC6)CC5)cc4c3)cnn2c1]

6-cyanopyrazolo[1,5-a]pyrimidine-3-carboxylic acid (13.0 mg, 69.1 umol, Intermediate BUW) was dissolved in DCM (1.5 mL) then TEA (20.9 mg, 207 umol) and (COCl)₂ (13.1 mg, 103 umol) was added dropwise to the solution at 0° C. The mixture was then stirred at 0° C. for 30 mins, then concentrated in vacuo to give crude acyl chloride. To a solution of 1-[8-[1-[[4-(5-aminoindazol-2-yl)cyclohexyl]methyl]-4-piperidyl]-4-isoquinolyl]hexahydropyrimidine-2,4-dione (46.0 mg, 69.1 umol, TFA, Intermediate BXP) in DCM (1.5 mL) was added TEA (13.9 mg, 138 umol) to adjust the pH to 8. Then the crude acyl chloride in DCM (1.5 mL) was added dropwise to the mixture and the reaction mixture was stirred at 0° C. for 30 mins. On completion, the mixture was quenched with H₂O (0.5 mL) and purified by pre-HPLC (column: Waters xbridge 150*25 mm 10 um; mobile phase: [water (10 mM NH₄HCO₃)-ACN]; B %: 19%-49%, 11 min) to give the title compound (1.30 mg, 3% yield) as light yellow solid. ¹H NMR (400 MHz, DMSO-d₆) δ 10.53 (s, 1H), 10.21 (d, J=2.0 Hz, 1H), 9.82 (s, 1H), 9.61 (s, 1H), 9.16 (d, J=2.0 Hz, 1H), 8.93 (s, 1H), 8.57 (s, 1H), 8.39 (s, 1H), 8.23 (d, J=2.0 Hz, 1H), 7.88-7.83 (m, 1H), 7.78 (t, J=8.0 Hz, 1H), 7.69-7.60 (m, 2H), 7.35 (dd, J=2.0, 10.0 Hz, 1H), 4.51-4.39 (m, 1H), 3.94-3.89 (m, 1H), 3.7-3.67 (m, 1H), 3.62-3.56 (m, 1H), 3.06-2.93 (m, 3H), 2.78-2.73 (m, 1H), 2.26-2.16 (m, 4H), 2.05-1.79 (m, 10H), 1.76-1.65 (m, 1H), 1.23-1.16 (m, 2H); LC-MS (ESI⁺) m/z 722.3 (M+H)⁺.

### Example 17: Synthesis of N-[2-[4-[[(1R,5S)-3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide (I-251) & N-[2-[4-[[(1S,5R)-3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide (I-250)

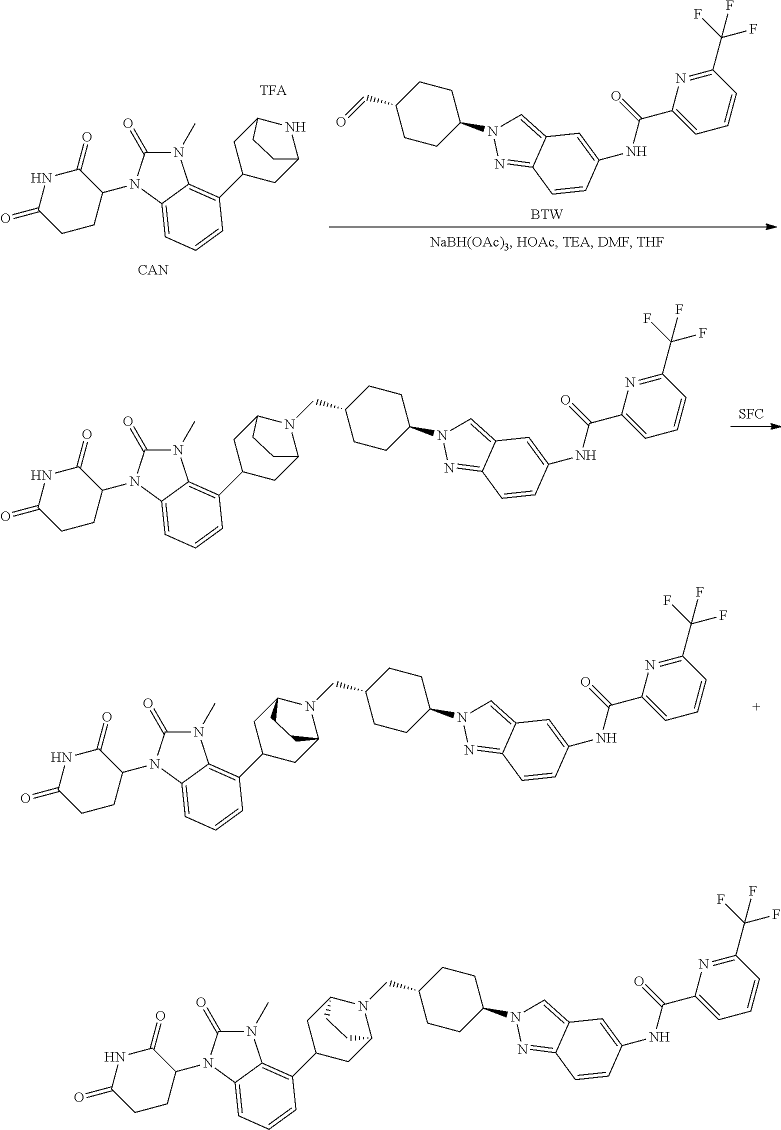

### Step 1—N-[2-[4-[[3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide

To a solution of 3-[4-(8-azabicyclo[3.2.1]octan-3-yl)-3-methyl-2-oxo-benzimidazol-1-yl]piperidine-2,6-dione (44 mg, 91.2 umol, TFA, Intermediate CAN), TEA (9.23 mg, 91.2 umol, 12.6 uL) HOAc (10.9 mg, 182 umol, 10.4 uL) in DMF (0.5 mL) and THF (0.5 mL) was added N-[2-(4-formylcyclohexyl)indazol-5-yl]-6-(trifluoromethyl)pyridine-2-carboxamide (37.9 mg, 91.2 umol, Intermediate BTW) at −10° C. The reaction mixture was stirred at −10° C. for 0.5 hr. Then, to the above reaction mixture was added NaBH(OAc)₃ (28.9 mg, 136 umol) at −10° C. and the mixture was stirred for 1.5 hrs. On completion, the reaction mixture was quenched with water (0.2 mL) and concentrated in vacuo. The residue was purified by prep-HPLC(column: Phenomenex luna C18 150*25 mm*10 um; mobile phase: [water(0.225% FA)-ACN]; B %: 20%-50%, 10 min) to give title compound (52 mg, 70% yield, FA) as white solid. ¹H NMR (400 MHz, DMSO-d₆) δ 11.11-11.07 (m, 1H), 10.36 (s, 1H), 8.44-8.33 (m, 3H), 8.32-8.29 (m, 1H), 8.19-8.13 (m, 1H), 7.64-7.59 (m, 1H), 7.57-7.53 (m, 1H), 7.17-6.92 (m, 3H), 5.40-5.33 (m, 1H), 4.52-4.40 (m, 1H), 3.88-3.78 (m, 1H), 3.59 (s, 3H), 2.93-2.83 (m, 1H), 2.76-2.67 (m, 1H), 2.65-2.59 (m, 1H), 2.45-2.37 (m, 2H), 2.29-2.17 (m, 4H), 2.12-1.86 (m, 9H), 1.65-1.14 (m, 7H).

### Step 2—N-[2-[4-[[(1R,5S)-3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide & N-[2-[4-[[(1S,5R)-3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide

N-(2-((1r, 4r)-4-((3-(1-(2,6-dioxopiperidin-3-yl)-3-methyl-2-oxo-2,3-dihydro-1H-benzo[d]imidazol-4-yl)-8-azabicyclo[3.2.1]octan-8-yl)methyl)cyclohexyl)-2H-indazol-5-yl)-6-(trifluoromethyl)picolinamide (52 mg) was separated by SFC (column: REGIS (R,R)WHELK-O1(250 mm*25 mm, 10 um); mobile phase: [0.1% NH3H2O ETOH]; B %: 60%-60%, 6; 60 min) to give N-[2-[4-[[(1R,5S)-3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide (30.86 mg, ¹H NMR (400 MHz, DMSO-d₆) δ 11.09 (s, 1H), 10.36 (s, 1H), 8.46-8.27 (m, 4H), 8.17 (d, J=7.6 Hz, 1H), 7.65-7.51 (m, 2H), 7.31-7.15 (m, 1H), 7.09-6.96 (m, 2H), 5.44-5.30 (m, 1H), 4.48 (d, J=3.6 Hz, 1H), 4.55-4.41 (m, 1H), 3.60 (s, 3H), 2.95-2.84 (m, 2H), 2.72-2.62 (m, 2H), 2.25-2.16 (m, 3H), 2.09-1.86 (m, 10H), 1.62-1.51 (m, 2H), 1.42-1.31 (m, 4H), 0.90-0.85 (m, 2H); LC-MS (ESI⁺) m/z 769.3 (M+H)⁺), the second of two peaks on SFC spectrogram) and N-[2-[4-[[(1S,5R)-3-[1-(2,6-dioxo-3-piperidyl)-3-methyl-2-oxo-benzimidazol-4-yl]-8-azabicyclo[3.2.1]octan-8-yl]methyl]cyclohexyl]indazol-5-yl]-6-(trifluoromethyl)pyridine-2-carboxamide (2.60 mg, ¹H NMR (400 MHz, DMSO-d₆) δ 11.09 (s, 1H), 10.36 (s, 1H), 8.44-8.28 (m, 4H), 8.20-8.15 (m, 1H), 7.65-7.52 (m, 2H), 7.16-6.94 (m, 3H), 5.41-5.32 (m, 1H), 4.53-4.43 (m, 1H), 3.82-3.70 (m, 1H), 3.66 (s, 3H), 2.92-2.83 (m, 1H), 2.77-2.59 (m, 3H), 2.24-2.15 (m, 3H), 2.11-1.90 (m, 10H), 1.81-1.58 (m, 3H), 1.35-1.12 (m, 4H), 0.91-0.80 (m, 1H)); LC-MS (ESI⁺) m/z 769.3 (M+H)⁺(The first of two peaks on SFC spectrogram). Absolute stereochemistry of the diastereomers was assigned arbitrarily.

### Example 18: Synthesis of N-[2-[4-[[4-[1-[(3S)-2,6-dioxo-3-piperidyl]-5-methoxy-3-methyl-2-oxo-benzimidazol-4-yl]-1-piperidyl]methyl]cyclohexyl]pyrazolo[3,4-c]pyridin-5-yl]-6-(trifluoromethyl)pyridine-2-carboxamide (I-275) & N-[2-[4-[[4-[1-[(3R)-2,6-dioxo-3-piperidyl]-5-methoxy-3-methyl-2-oxo-benzimidazol-4-yl]-1-piperidyl]methyl]cyclohexyl]pyrazolo[3,4-c]pyridine-5-yl]-6-(trifluoromethyl)pyridine-2-carboxamide (I-274)

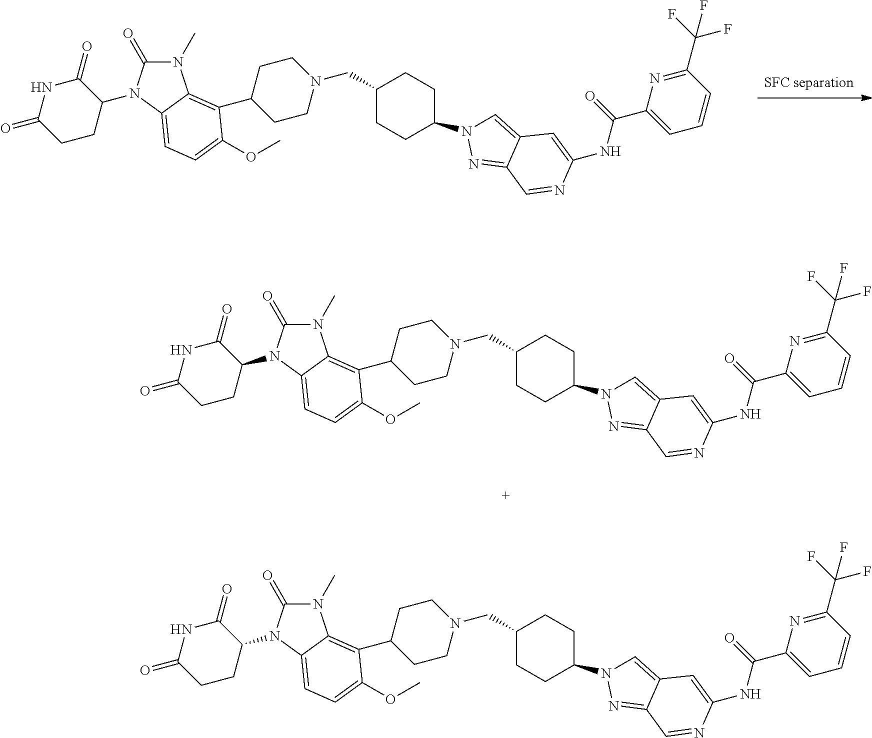 [SMILES: COc1ccc2c(c1C1CCN(C[C@H]3CC[C@H](n4cc5cc(NC(=O)c6cccc(C(F)(F)F)n6)ncc5n4)CC3)CC1)n(C)c(=O)n2C1CCC(=O)NC1=O ; "SFC separation" ; COc1ccc2c(c1C1CCN(C[C@H]3CC[C@H](n4cc5cc(NC(=O)c6cccc(C(F)(F)F)n6)ncc5n4)CC3)CC1)n(C)c(=O)n2[C@H]1CCC(=O)NC1=O ; COc1ccc2c(c1C1CCN(C[C@H]3CC[C@H](n4cc5cc(NC(=O)c6cccc(C(F)(F)F)n6)ncc5n4)CC3)CC1)n(C)c(=O)n2[C@@H]1CCC(=O)NC1=O]

N-[2-[4-[[4-[1-[(3S)-2,6-dioxo-3-piperidyl]-5-methoxy-3-methyl-2-oxo-benzimidazol-4-yl]-1-piperidyl]methyl]cyclohexyl]pyrazolo[3,4-c]pyridin-5-yl]-6-(trifluoromethyl)pyridine-2-carboxamide (300 mg, 387 umol, Example I-104 was separated by SFC (column: DAICEL CHIRALPAK IC (250 mm*30 mm, 10 um); mobile phase: [IPA-ACN]; B %: 80%-80%, 6.8; 30 min) to give N-[2-[4-[[4-[1-[(3S)-2,6-dioxo-3-piperidyl]-5-methoxy-3-methyl-2-oxo-benzimidazol-4-yl]-1-piperidyl]methyl]cyclohexyl]pyrazolo[3,4-c]pyridin-5-yl]-6-(trifluoromethyl)pyridine-2-carboxamide (peak 1, 23.3 mg, 7% yield, FA) as a white solid (¹H NMR (400 MHz, DMSO-d₆) δ 11.08 (s, 1H), 10.20 (s, 1H), 9.07 (s, 1H), 8.62 (s, 1H), 8.52-8.46 (m, 2H), 8.45-8.38 (m, 1H), 8.23 (d, J=7.6 Hz, 1H), 6.91 (d, J=8.4 Hz, 1H), 6.72 (d, J=8.4 Hz, 1H), 5.32 (dd, J=5.2, 13.2 Hz, 1H), 4.62-4.54 (m, 1H), 3.77 (s, 3H), 3.56 (s, 3H), 2.97 (d, J=10.0 Hz, 2H), 2.92-2.82 (m, 1H), 2.69 (s, 2H), 2.41 (d, J=12.0 Hz, 3H), 2.20 (d, J=6.4 Hz, 4H), 1.98 (t, J=10.0 Hz, 7H), 1.74-1.65 (m, 1H), 1.62-1.53 (m, 2H), 1.24-1.11 (m, 2H); LC-MS (ESI⁺) m/z 774.2 (M+H)⁺) and N-[2-[4-[[4-[1-[(3R)-2,6-dioxo-3-piperidyl]-5-methoxy-3-methyl-2-oxo-benzimidazol-4-yl]-1-piperidyl]methyl]cyclohexyl]pyrazolo[3,4-c]pyridine-5-yl]-6-(trifluoromethyl)pyridine-2-carboxamide (peak 2, 9.8 mg, 5% yield, FA) as a white solid (¹H NMR (400 MHz, DMSO-d₆) δ 11.08 (s, 1H), 10.20 (s, 1H), 9.07 (s, 1H), 8.61 (s, 1H), 8.52-8.46 (m, 2H), 8.45-8.38 (m, 1H), 8.23 (d, J=7.6 Hz, 1H), 6.91 (d, J=8.8 Hz, 1H), 6.72 (d, J=8.8 Hz, 1H), 5.32 (m, 1H), 4.66-4.51 (m, 1H), 3.76 (s, 3H), 3.56 (s, 3H), 2.97 (d, J=10.8 Hz, 2H), 2.92-2.80 (m, 1H), 2.71-2.58 (m, 4H), 2.41 (d, J=11.2 Hz, 2H), 2.20 (d, J=8.0 Hz, 4H), 1.99 (t, J=10.0 Hz, 6H), 1.75-1.64 (m, 1H), 1.57 (d, J=10.8 Hz, 2H), 1.24-1.08 (m, 2H); LC-MS (ESI⁺) m/z 774.3 (M+H)⁺).

### Example 19: Synthesis of N-[2-[4-[[[2-(2,6-dioxo-3-piperidyl)-1,3-dioxo-isoindolin-4-yl]amino]methyl]cyclohexyl]-6-(1-hydroxy-1-methyl-ethyl)indazol-5-yl]-6-(trifluoromethyl)pyridine-2-carboxamide (I-338)

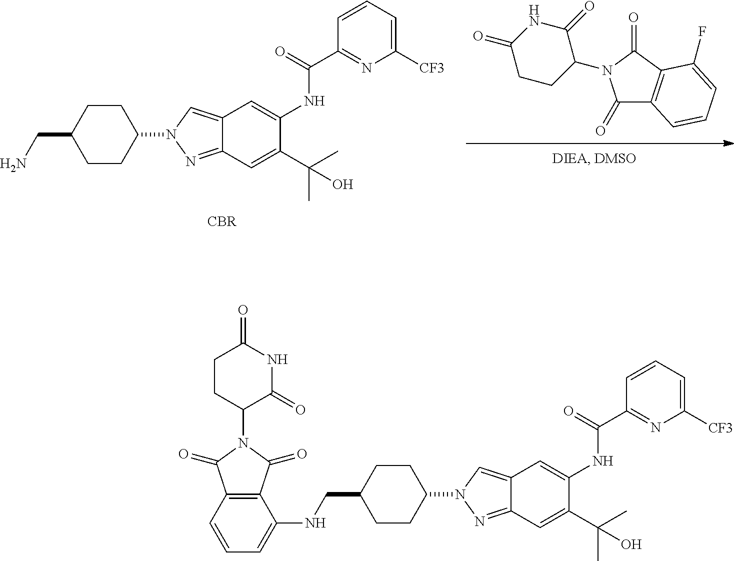 [SMILES: CC(C)(O)c1cc2nn([C@H]3CC[C@H](CN)CC3)cc2cc1NC(=O)c1cccc(C(F)(F)F)n1 ; O=C1CCC(N2C(=O)c3cccc(F)c3C2=O)C(=O)N1 ; "CBR" ; CC(C)(O)c1cc2nn([C@H]3CC[C@H](CNc4cccc5c4C(=O)N(C4CCC(=O)NC4=O)C5=O)CC3)cc2cc1NC(=O)c1cccc(C(F)(F)F)n1]

A mixture of N-[2-[4-(aminomethyl)cyclohexyl]-6-(1-hydroxy-1-methyl-ethyl)indazol-5-yl]-6-(trifluoromethyl)pyridine-2-carboxamide (15.0 mg, 31.5 umol, Intermediate CBR), 2-(2,6-dioxo-3-piperidyl)-4-fluoro-isoindoline-1,3-dione (7.26 mg, 26.2 umol, CAS #835616-60-9), DIEA (3.40 mg, 26.2 umol) in DMSO (2 mL) was degassed and purged with N₂ three times. Then the mixture was stirred at 100° C. for 1 hours under N₂ atmosphere. On completion, the crude product was purified by reversed-phase HPLC (column: Phenomenex Luna C18 100*30 mm*5 um; mobile phase: [water(FA)-ACN]; B %: 47%-77%, 8 min) to give the title compound (3.91 mg, 20% yield) as yellow solid. LC-MS (ESI⁺) m/z 732.2 (M+H)⁺¹. 1H NMR (400 MHz, DMSO-d6) δ 12.36 (s, 1H), 11.18-11.02 (m, 1H), 8.71 (s, 1H), 8.49-8.43 (m, 1H), 8.41-8.33 (m, 2H), 8.16 (d, J=7.8 Hz, 1H), 7.66-7.54 (m, 2H), 7.19 (d, J=8.6 Hz, 1H), 7.05 (d, J=7.0 Hz, 1H), 6.69 (t, J=6.0 Hz, 1H), 5.94 (s, 1H), 5.07 (dd, J=5.4, 12.9 Hz, 1H), 4.53-4.39 (m, 1H), 2.96-2.82 (m, 1H), 2.62 (d, J=2.6 Hz, 1H), 2.57 (d, J=8.1 Hz, 1H), 2.53 (s, 2H), 2.53 (s, 4H), 2.17 (d, J=10.3 Hz, 2H), 2.09-2.03 (m, 1H), 1.98 (s, 1H), 1.94 (s, 2H), 1.92-1.87 (m, 1H), 1.81-1.70 (m, 1H), 1.62 (s, 6H), 1.37-1.11 (m, 3H).

### Example 20: Synthesis of N-[2-[4-[[2-[[2-(2,6-dioxo-3-piperidyl)-1,3-dioxo-isoindolin-4-yl]amino]ethyl-methyl-amino]methyl]cyclohexyl]-6-(1-hydroxy-1-methyl-ethyl)indazol-5-yl]-6-(trifluoromethyl)pyridine-2-carboxamide (I-341)

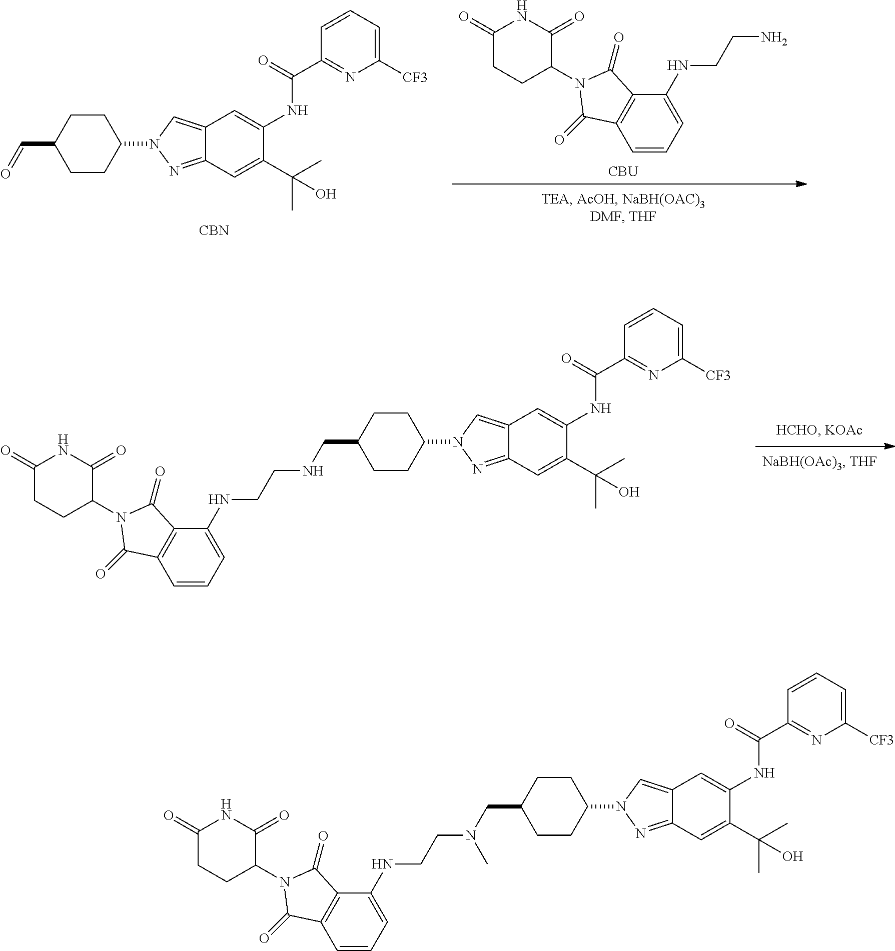

### Step 1—N-[2-[4-[[2-[[2-(2,6-dioxo-3-piperidyl)-1,3-dioxo-isoindolin-4-yl]amino]ethylamino]methyl] cyclohexyl]-6-(1-hydroxy-1-methyl-ethyl)indazol-5-yl]-6-(trifluoromethyl)pyridine-2-carboxamide

To a mixture of 4-(2-aminoethylamino)-2-(2,6-dioxo-3-piperidyl)isoindoline-1,3-dione (113 mg, 357 umol Intermediate CBU) in a mixed solvent of THF (2.0 mL) and DMF (0.5 mL) was added Et₃N (36.1 mg, 357 umol, 49.7 uL) at −10° C. until the pH stabilized at 8. The mixture was stirred at −10° C. for 2 minutes, then HOAc (21.4 mg, 357 umol, 20.4 uL) was added at −10° C. to solution until the pH stabilized at 5-6. Subsequently, N-[2-(4-formylcyclohexyl)-6-(1-hydroxy-1-methyl-ethyl)indazol-5-yl]-6-(trifluoromethyl)pyridine-2-carboxamide (135 mg, 285 umol, Intermediate CBN) was added and stirred for 1 hour. After that, NaBH(OAc)₃ (151 mg, 714 umol) was added in one portion. The resulting reaction mixture was stirred at −10° C. for 1 hour. On completion, the mixture was concentrated to give a residue. The residue was purified by prep-HPLC (column: Phenomenex luna C18 150*25 mm*10 um; mobile phase: [water(FA)-ACN]; B %: 20%-50%, 10.5 min) to give the title compound (57.1 mg, 20% yield) as a yellow solid. ¹HNMR (400 MHz, DMSO-d₆) δ 12.36 (s, 1H), 11.09 (s, 1H), 8.71 (s, 1H), 8.47-8.42 (m, 1H), 8.41-8.34 (m, 2H), 8.16 (d, J=8.0 Hz, 1H), 7.63 (t, J=8.0 Hz, 1H), 7.57 (s, 1H), 7.16 (d, J=8.8 Hz, 1H), 7.08 (d, J=7.2 Hz, 1H), 6.86 (t, J=5.2 Hz, 1H), 5.93 (s, 1H), 5.12-5.01 (m, 1H), 4.52-4.36 (m, 1H), 3.57-3.47 (m, 2H), 2.98 (s, 2H), 2.91-2.84 (m, 1H), 2.70 (d, J=1.2 Hz, 1H), 2.61 (d, J=2.0 Hz, 2H), 2.16 (d, J=12.0 Hz, 2H), 2.08-1.83 (m, 6H), 1.62 (s, 8H), 1.29-1.16 (m, 2H). LC-MS (ESI+) m/z 775.2 (M+H)⁺.

### Step 2—N-[2-[4-[[2-[[2-(2,6-dioxo-3-piperidyl)-1,3-dioxo-isoindolin-4-yl]amino]ethyl-methyl-amino]methyl]cyclohexyl]-6-(1-hydroxy-1-methyl-ethyl)indazol-5-yl]-6-(trifluoromethyl)pyridine-2-carboxamide

To a solution of N-[2-[4-[[2-[[2-(2,6-dioxo-3-piperidyl)-1,3-dioxo-isoindolin-4-yl]amino]ethylamino]methyl]cyclohexyl]-6-(1-hydroxy-1-methyl-ethyl)indazol-5-yl]-6-(trifluoromethyl)pyridine-2-carboxamide (20.0 mg, 25.8 umol) in THF (0.5 mL) was added, and HCHO (13.9 mg, 464 umol, 12.8 uL) at 0° C. was stirred for 0.25 hours. Next, NaBH(OAc)₃ (10.9 mg, 51.6 umol) was added in portions at 0° C. The mixture was then stirred at 20° C. for 2 hours. On completion, the mixture was concentrated to give a residue. The residue was purified by prep-HPLC (column: Phenomenex luna C18 150*25 mm*10 um; mobile phase: [water (FA)-ACN]; B %: 18%-48%, 10.5 min) to give the title compound (11.3 mg, 55% yield) as a yellow solid. ¹H NMR (400 MHz, DMSO-d₆) δ 12.36 (s, 1H), 11.06 (s, 1H), 8.72-8.67 (m, 1H), 8.48-8.43 (m, 1H), 8.39-8.28 (m, 2H), 8.16 (d, J=8.0 Hz, 1H), 7.64-7.54 (m, 2H), 7.11 (d, J=8.4 Hz, 1H), 7.04 (d, J=7.2 Hz, 1H), 6.79-6.72 (m, 1H), 5.95-5.92 (m, 1H), 5.06-4.99 (m, 1H), 4.40 (tt, J=4.0, 12.0 Hz, 1H), 3.36 (d, J=5.2 Hz, 2H), 2.89-2.78 (m, 1H), 2.59 (t, J=5.6 Hz, 2H), 2.56-2.52 (m, 2H), 2.24-2.21 (m, 4H), 2.11 (d, J=11.6 Hz, 2H), 2.04-1.92 (m, 3H), 1.90-1.81 (m, 2H), 1.62 (s, 8H), 1.16-1.02 (m, 2H); LC-MS (ESI+) m/z 789.3 (M+H)⁺.

### Examples 21: Syntheses of N-[2-[4-[[4-[1-[(3S)-2,6-dioxo-3-piperidyl]-3-methyl-2-oxo-benzimidazol-4-yl]-1-piperidyl]methyl]cyclohexyl]-6-methyl-indazol-5-yl]-6-(trifluoromethyl)pyridine-2-carboxamide (I-390) and N-[2-[4-[[4-[1-[(3R)-2,6-dioxo-3-piperidyl]-3-methyl-2-oxo-benzimidazol-4-yl]-1-piperidyl]methyl]cyclohexyl]-6-methyl-indazol-5-yl]-6-(trifluoromethyl)pyridine-2-carboxamide (I-394)

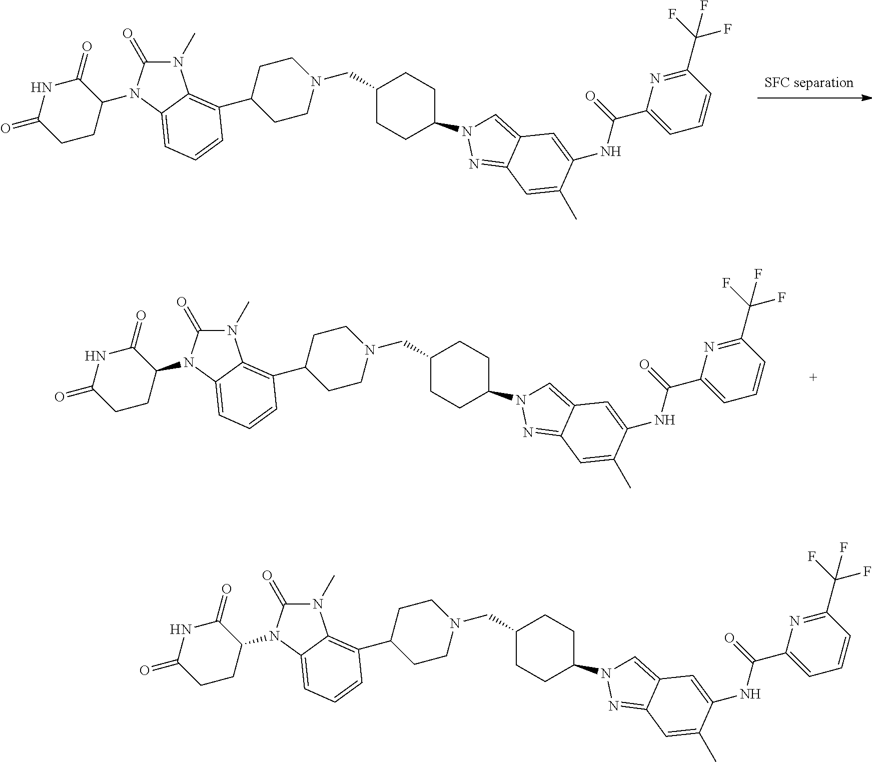 [SMILES: Cc1cc2nn([C@H]3CC[C@H](CN4CCC(c5cccc6c5n(C)c(=O)n6C5CCC(=O)NC5=O)CC4)CC3)cc2cc1NC(=O)c1cccc(C(F)(F)F)n1 ; "SFC separation" ; Cc1cc2nn([C@H]3CC[C@H](CN4CCC(c5cccc6c5n(C)c(=O)n6[C@H]5CCC(=O)NC5=O)CC4)CC3)cc2cc1NC(=O)c1cccc(C(F)(F)F)n1 ; Cc1cc2nn([C@H]3CC[C@H](CN4CCC(c5cccc6c5n(C)c(=O)n6[C@@H]5CCC(=O)NC5=O)CC4)CC3)cc2cc1NC(=O)c1cccc(C(F)(F)F)n1]

N-[2-[4-[[4-[1-(2,6-dioxo-3-piperidyl)-3-methyl-2-oxo-benzimidazol-4-yl]-1-piperidyl]methyl]cyclohexyl]-6-methyl-indazol-5-yl]-6-(trifluoromethyl)pyridine-2-carboxamide (500 mg, 660 umol, synthesized via Method 2 coupling Intermediate AZK with Intermediate BSC) was purified by SFC (column: Welch Ultimate C18 150*25 mm*5 um; mobile phase: [water (FA)-ACN]; B %: 18%-48%, 10 min) to give N-[2-[4-[[4-[1-[(3S)-2,6-dioxo-3-piperidyl]-3-methyl-2-oxo-benzimidazol-4-yl]-1-piperidyl]methyl]cyclohexyl]-6-methyl-indazol-5-yl]-6-(trifluoromethyl)pyridine-2-carboxamide (119 mg, 24% yield) as a white solid (¹HNMR(400 MHz, DMSO-d₆) δ 11.10 (s, 1H), 10.13 (s, 1H), 8.45-8.42 (m, 1H), 8.41-8.36 (m, 2H), 8.22-8.19 (m, 2H), 7.51 (s, 1H), 7.05-7.00 (m, 2H), 6.99-6.96 (m, 1H), 5.41-5.33 (m, 1H), 4.48-4.38 (m, 1H), 3.59 (s, 3H), 3.03-2.97 (m, 2H), 2.94-2.84 (m, 1H), 2.77-2.65 (m, 2H), 2.54 (s, 1H), 2.40 (s, 3H), 2.25-2.13 (m, 4H), 2.12-2.03 (m, 2H), 2.02-1.89 (m, 5H), 1.84-1.75 (m, 4H), 1.72-1.61 (m, 1H), 1.20-1.08 (m, 2H). LC-MS (ESI⁺) m/z 757.3 (M+H)⁺) and N-[2-[4-[[4-[1-[(3R)-2,6-dioxo-3-piperidyl]-3-methyl-2-oxo-benzimidazol-4-yl]-1-piperidyl]methyl]cyclohexyl]-6-methyl-indazol-5-yl]-6-(trifluoromethyl)pyridine-2-carboxamide (71.0 mg, 14% yield) as a white solid (¹H NMR (400 MHz, DMSO-d₆) δ 11.10 (s, 1H), 10.13 (s, 1H), 8.45-8.42 (m, 1H), 8.40 (d, J=7.6 Hz, 1H), 8.37 (s, 1H), 8.23-8.17 (m, 2H), 7.51 (s, 1H), 7.05-6.95 (m, 3H), 5.37-5.34 (m, 1H), 4.48-4.38 (m, 1H), 3.59 (s, 3H), 3.00-2.99 (m, 2H), 2.94-2.84 (m, 1H), 2.76-2.66 (m, 2H), 2.40 (s, 3H), 2.24-2.13 (m, 4H), 2.11-2.04 (m, 2H), 2.03-1.90 (m, 5H), 1.84-1.74 (m, 4H), 1.71-1.62 (m, 1H), 1.23 (s, 1H), 1.20-1.08 (m, 2H). LC-MS (ESI⁺) m/z 757.3 (M+H)⁺). The absolute stereochemistry was assigned arbitrarily.

### Example 22: Syntheses of N-[2-[4-[[4-[1-[(3S)-2,6-dioxo-3-piperidyl]-3-methyl-2-oxo-benzimidazol-4-yl]piperazin-1-yl]methyl]cyclohexyl]-6-methyl-indazol-5-yl]-6-(trifluoromethyl)pyridine-2-carboxamide (I-399) and N-[2-[4-[[4-[1-[(3R)-2,6-dioxo-3-piperidyl]-3-methyl-2-oxo-benzimidazol-4-yl]piperazin-1-yl]methyl]cyclohexyl]-6-methyl-indazol-5-yl]-6-(trifluoromethyl)pyridine-2-carboxamide (I-400)

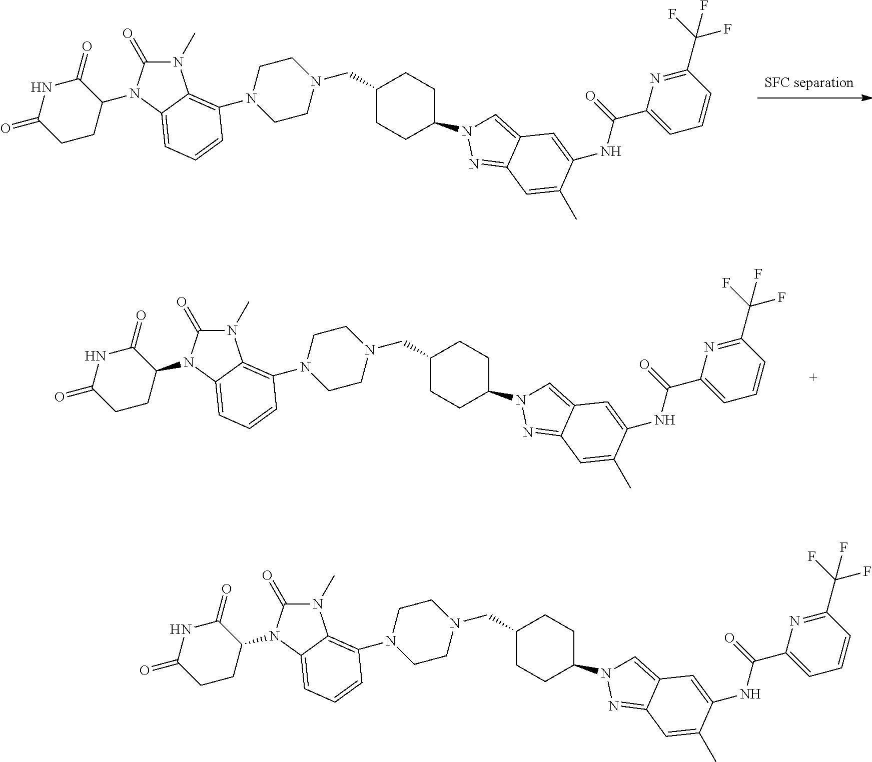 [SMILES: Cc1cc2nn([C@H]3CC[C@H](CN4CCN(c5cccc6c5n(C)c(=O)n6C5CCC(=O)NC5=O)CC4)CC3)cc2cc1NC(=O)c1cccc(C(F)(F)F)n1 ; "SFC separation" ; Cc1cc2nn([C@H]3CC[C@H](CN4CCN(c5cccc6c5n(C)c(=O)n6[C@H]5CCC(=O)NC5=O)CC4)CC3)cc2cc1NC(=O)c1cccc(C(F)(F)F)n1 ; Cc1cc2nn([C@H]3CC[C@H](CN4CCN(c5cccc6c5n(C)c(=O)n6[C@@H]5CCC(=O)NC5=O)CC4)CC3)cc2cc1NC(=O)c1cccc(C(F)(F)F)n1]

N-[2-[4-[[4-[1-(2,6-dioxo-3-piperidyl)-3-methyl-2-oxo-benzimidazol-4-yl]piperazin-1-yl]methyl]cyclohexyl]-6-methyl-indazol-5-yl]-6-(trifluoromethyl)pyridine-2-carboxamide (500 mg, 660 umol, Example I-260) was separated by SFC (column: DAICEL CHIRALPAK AD(250 mm*30 mm, 10 um); mobile phase: [0.1% NH3H2O IPA]; B %: 60%-60%, 5; 80 min) to give N-[2-[4-[[4-[1-[(3S)-2,6-dioxo-3-piperidyl]-3-methyl-2-oxo-benzimidazol-4-yl]piperazin-1-yl]methyl]cyclohexyl]-6-methyl-indazol-5-yl]-6-(trifluoromethyl)pyridine-2-carboxamide (175 mg, 35% yield) as a yellow solid (¹H NMR (400 MHz, DMSO-d₆) δ 11.09 (s, 1H), 10.13 (s, 1H), 8.46-8.34 (m, 3H), 8.23-8.18 (m, 2H), 7.51 (s, 1H), 7.02-6.92 (m, 2H), 6.89 (d, J=7.6 Hz, 1H), 5.37-5.32 (m, 1H), 4.50-4.37 (m, 1H), 3.64 (s, 3H), 3.02-2.84 (m, 6H), 2.66-2.58 (m, 2H), 2.56-2.53 (m, 3H), 2.40 (s, 3H), 2.26-2.25 (m, 2H), 2.18-2.15 (m, 2H), 2.04-1.88 (m, 5H), 1.75-1.62 (m, 1H), 1.23-1.08 (m, 2H); LC-MS (ESI⁺) m/z 758.3 (M+H)⁺) and N-[2-[4-[[4-[1-[(3R)-2,6-dioxo-3-piperidyl]-3-methyl-2-oxo-benzimidazol-4-yl]piperazin-1-yl]methyl]cyclohexyl]-6-methyl-indazol-5-yl]-6-(trifluoromethyl)pyridine-2-carboxamide (175 mg, 35% yield) as a yellow solid (¹H NMR (400 MHz, DMSO-d₆) δ 11.09 (s, 1H), 10.13 (s, 1H), 8.46-8.35 (m, 3H), 8.23-8.18 (m, 2H), 7.51 (s, 1H), 7.02-6.92 (m, 2H), 6.89 (d, J=7.2 Hz, 1H), 5.37-5.32 (m, 1H), 4.49-4.38 (m, 1H), 3.64 (s, 3H), 3.01-2.82 (m, 6H), 2.67-2.56 (m, 2H), 2.52-2.51 (m, 3H), 2.40 (s, 3H), 2.26-2.24 (m, 2H), 2.17-2.15 (m, 2H), 2.03-1.87 (m, 5H), 1.74-1.62 (m, 1H), 1.24-1.08 (m, 2H). LC-MS (ESI⁺) m/z 758.3 (M+H)⁺).

### Example 23: Synthesis of 2-[4-[[4-[1-(2,6-dioxo-3-piperidyl)-3-methyl-2-oxo-benzimidazol-4-yl]piperazin-1-yl]methyl]cyclohexyl]-5-[[6-(trifluoromethyl)pyridine-2-carbonyl]amino]indazole-6-carboxylic acid (I-415)

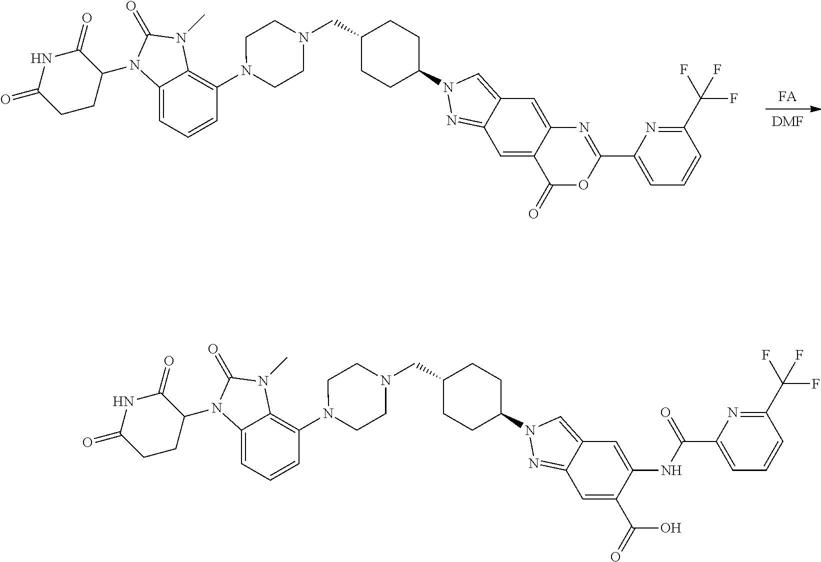

To a solution of 3-[3-methyl-2-oxo-4-[4-[[4-[8-oxo-6-[6-(trifluoromethyl)-2-pyridyl]pyrazolo[3,4-g]benzoxazin-2-yl]cyclohexyl]methyl]piperazin-1-yl]benzimidazol-1-yl]piperidine-2,6-dione (25.0 mg, 32.4 umol, Example I-406) in DMF (0.5 mL) was added FA (0.2 mL) at 25° C. The reaction was then stirred at 25° C. for 48 hr. On completion, the reaction mixture was filtered and the filtrate was concentrated in vacuo. The crude product was purified by prep-HPLC (column: Phenomenex Luna C18 150*25 mm*10 um; mobile phase: [water (FA)-ACN]; B %: 15%-45%, 10.5 min) to give the title compound (8.7 mg, 34% yield) as white solid. ¹H NMR (400 MHz, DMSO-d₆) δ 11.08 (s, 1H), 9.10 (s, 1H), 8.49 (s, 1H), 8.46-8.41 (m, 2H), 8.36 (t, J=8.0 Hz, 1H), 8.16 (d, J=7.6 Hz, 1H), 7.02-6.93 (m, 2H), 6.91-6.87 (m, 1H), 5.39-5.31 (m, 1H), 4.55-4.46 (m, 1H), 3.64 (s, 3H), 3.07-2.83 (m, 7H), 2.70 (s, 1H), 2.67-2.58 (m, 2H), 2.27 (d, J=6.8 Hz, 3H), 2.22-2.15 (m, 3H), 2.05-1.91 (m, 6H), 1.75-1.65 (m, 1H), 1.23-1.14 (m, 2H); LC-MS (ESI⁺) m/z 788.3 (M+H)⁺.

### Example 24. IRAK4 MSD Degradation in OCI-LY10

Degradation of IRAK4 in OCI-LY10 was quantitatively measured using Meso Scale Discovery technology. OCI-LY10 cells were seeded in 96-well plates (Corning 3799) with a density of 300,000 cells per well in 100 μL fresh media. Compounds were then added to the assay plates with a final top concentration of 1 to 10 μM in a 1:3 dilution series with total of 8 doses. The assay plates were then incubated for 4 to 24 hours at 37° C. under 5% CO2. The assay plates were then centrifuged for 5 minutes and the cell pellets were treated with 100 μL/well RIPA lysis buffer (Boston BioProducts BP-115D) with proteinase inhibitors. To prepare MSD assay plates (Meso Scale Discovery Catalog number L15XA-3), the plates were coated with 2 μg/mL capture antibody (mouse Anti-IRAK4 antibody [2H9], ab119942) in PBS, at 40 μL/well. The plates were then incubated overnight at 4° C., washed 3 times with 150 μL/well TBST buffer (Cell Signaling Technology, Catalog number 9997S) and blocked with 150 L/well blocking buffer (Meso Scale Discovery Catalog number R93BA-4). Cell lysates were then added to MSD assay plates and the plates were incubated at room temperature for 1 hour. The plates were then washed 3 times with 150 μL/well TBST buffer and 25 μL/well primary detection antibody (rabbitAnti-IRAK4 antibody [Y279], from Abcam. Catalog number ab32511, 1 μg/mL). The assay plates were then incubated at room temperature for 1 hour, washed 3 times with 150 μL/well TBST buffer and 25 μL/well secondary detection antibody, SULFO-TAG anti-rabbit antibody were added (anti rabbit antibody from Meso Scale Discovery, Catalog number R32AB-1, 1 μg/mL). The assay plates were then incubated at room temperature for 1 hour, washed 3 times with 150 μL/well TBST buffer, and 150 μL/well MSD reading buffer (Meso Scale Discovery catalog number R92TC-2) was added. The plates were then analyzed by a MSD reader (Meso Scale Discovery, Model Quick Plex SQ 120). The data was then analyzed by software Prism 7.0 from GraphPad and the dose-depended IRAK4 degradation were fit using a three-parameter logistic equation to calculate DC₅₀.

IRAK4 MSD degradation results in OCI-LY10 cells for compounds of the invention are presented in Table 6. The letter codes for IRAK4 DC₅₀ include: A (<0.01 μM); B (0.01-0.05 μM); C (0.05-0.1 μM); D (0.1-0.2 μM); and E (>0.2 μM).

TABLE 6
IRAK4 MSD Degradation in OCI-LY10 Results

| I-# | IRAK4 degradation in OCI-LY10: DC₅₀ (μM) |
|---|---|
| I-1 | E |
| I-2 | E |
| I-3 | C |
| I-4 | C |
| I-5 | A |
| I-6 | A |
| I-7 | E |
| I-8 | B |
| I-9 | C |
| I-10 | C |
| I-11 | C |
| I-12 | B |
| I-13 | A |
| I-14 | C |
| I-15 | E |
| I-16 | B |
| I-17 | A |
| I-18 | E |
| I-19 | E |
| I-20 | E |
| I-21 | D |
| I-22 | E |
| I-23 | D |
| I-24 | B |
| I-25 | A |
| I-26 | A |
| I-27 | A |
| I-28 | B |
| I-29 | A |
| I-30 | B |
| I-31 | A |
| I-32 | A |
| I-33 | A |
| I-34 | E |
| I-35 | A |
| I-36 | A |
| I-37 | A |
| I-38 | A |
| I-39 | A |
| I-40 | B |
| I-41 | E |
| I-43 | E |
| I-44 | A |
| I-45 | A |
| I-46 | B |
| I-47 | B |
| I-48 | A |
| I-49 | E |
| I-50 | B |
| I-51 | A |
| I-52 | A |
| I-53 | B |
| I-54 | A |
| I-55 | B |
| I-56 | B |
| I-57 | A |
| I-58 | A |
| I-59 | E |
| I-60 | E |
| I-61 | E |
| I-62 | A |
| I-63 | A |
| I-64 | A |
| I-65 | A |
| I-66 | A |
| I-67 | B |
| I-68 | B |
| I-69 | A |
| I-70 | A |
| I-71 | A |
| I-72 | B |
| I-73 | B |
| I-74 | B |
| I-75 | B |
| I-76 | B |
| I-77 | A |
| I-78 | A |
| I-79 | E |
| I-80 | A |
| I-81 | A |
| I-82 | A |
| I-83 | A |
| I-84 | C |
| I-85 | C |
| I-86 | A |
| I-87 | A |
| I-88 | B |
| I-89 | B |
| I-90 | B |
| I-91 | B |
| I-92 | A |
| I-93 | A |
| I-94 | E |
| I-95 | A |
| I-96 | A |
| I-97 | A |
| I-98 | E |
| I-99 | A |
| I-100 | E |
| I-101 | A |
| I-102 | A |
| I-103 | B |
| I-104 | A |
| I-105 | C |
| I-106 | B |
| I-107 | A |
| I-108 | A |
| I-109 | A |
| I-110 | A |
| I-111 | A |
| I-112 | A |
| I-113 | A |
| I-114 | A |
| I-115 | E |
| I-116 | D |
| I-117 | C |
| I-118 | A |
| I-119 | E |
| I-120 | E |
| I-121 | B |
| I-122 | D |
| I-123 | B |
| I-125 | A |
| I-126 | B |
| I-127 | B |
| I-128 | A |
| I-129 | A |
| I-130 | A |
| I-131 | B |
| I-132 | A |
| I-134 | E |
| I-135 | E |
| I-136 | E |
| I-137 | A |
| I-138 | A |
| I-139 | B |
| I-140 | D |
| I-141 | B |
| I-142 | B |
| I-143 | C |
| I-144 | C |
| I-145 | B |
| I-147 | C |
| I-148 | D |
| I-149 | D |
| I-150 | D |
| I-151 | A |
| I-152 | B |
| I-154 | A |
| I-155 | B |
| I-156 | E |
| I-157 | C |
| I-158 | A |
| I-159 | A |
| I-160 | E |
| I-161 | E |
| I-162 | E |
| I-163 | A |
| I-164 | A |
| I-165 | A |
| I-166 | E |
| I-167 | A |
| I-168 | A |
| I-169 | E |
| I-170 | E |
| I-171 | B |
| I-172 | E |
| I-173 | A |
| I-174 | A |
| I-175 | A |
| I-176 | A |
| I-177 | A |
| I-178 | E |
| I-179 | A |
| I-180 | E |
| I-181 | E |
| I-182 | A |
| I-183 | A |
| I-184 | A |
| I-185 | A |
| I-186 | A |
| I-187 | A |
| I-188 | E |
| I-189 | A |
| I-190 | A |
| I-191 | B |
| I-193 | E |
| I-194 | E |
| I-195 | B |
| I-196 | A |
| I-197 | A |
| I-198 | A |
| I-199 | B |
| I-200 | A |
| I-201 | E |
| I-202 | E |
| I-204 | B |
| I-205 | A |
| I-206 | A |
| I-207 | A |
| I-208 | A |
| I-209 | A |
| I-210 | E |
| I-211 | A |
| I-212 | E |
| I-213 | A |
| I-214 | A |
| I-215 | A |
| I-216 | A |
| I-217 | E |
| I-218 | A |
| I-219 | A |
| I-220 | A |
| I-221 | A |
| I-222 | A |
| I-223 | B |
| I-224 | A |
| I-225 | A |
| I-226 | A |
| I-227 | A |
| I-228 | A |
| I-229 | A |
| I-230 | A |
| I-231 | B |
| I-232 | A |
| I-233 | A |
| I-234 | A |
| I-235 | C |
| I-237 | B |
| I-238 | A |
| I-239 | A |
| I-240 | A |
| I-241 | E |
| I-242 | A |
| I-243 | A |
| I-244 | D |
| I-245 | E |
| I-246 | B |
| I-247 | E |
| I-248 | A |
| I-249 | E |
| I-250 | B |
| I-251 | A |
| I-252 | B |
| I-253 | A |
| I-254 | A |
| I-255 | A |
| I-256 | A |
| I-257 | A |
| I-258 | E |
| I-259 | B |
| I-261 | B |
| I-262 | A |
| I-263 | B |
| I-266 | A |
| I-267 | E |
| I-268 | A |
| I-269 | A |
| I-271 | A |
| I-272 | A |
| I-273 | A |
| I-274 | B |
| I-275 | A |
| I-276 | A |
| I-277 | B |
| I-278 | A |
| I-279 | B |
| I-280 | B |
| I-281 | B |
| I-282 | A |
| I-283 | D |
| I-284 | B |
| I-285 | A |
| I-286 | B |
| I-287 | A |
| I-288 | B |
| I-289 | E |
| I-290 | B |
| I-291 | B |
| I-292 | A |
| I-293 | A |
| I-294 | B |
| I-295 | B |
| I-296 | B |
| I-297 | A |
| I-298 | A |
| I-299 | A |
| I-300 | B |
| I-301 | A |
| I-302 | B |
| I-303 | A |
| I-304 | B |
| I-305 | B |
| I-306 | B |
| I-307 | A |
| I-308 | A |
| I-309 | A |
| I-310 | E |
| I-311 | A |
| I-312 | E |
| I-313 | E |
| I-317 | D |
| I-318 | E |
| I-319 | B |
| I-320 | A |
| I-321 | E |
| I-322 | A |
| I-323 | A |
| I-324 | B |
| I-325 | E |
| I-326 | A |
| I-327 | A |
| I-328 | B |
| I-329 | A |
| I-330 | E |
| I-331 | A |
| I-332 | B |
| I-353 | E |
| I-354 | E |
| I-355 | A |
| I-356 | A |
| I-357 | A |
| I-358 | B |
| I-359 | A |
| I-360 | B |
| I-361 | A |
| I-362 | A |
| I-363 | A |
| I-364 | A |
| I-365 | A |
| I-366 | A |
| I-367 | B |
| I-368 | E |
| I-369 | D |
| I-370 | B |
| I-371 | A |
| I-372 | A |
| I-373 | B |
| I-374 | E |
| I-375 | B |
| I-376 | B |
| I-377 | A |
| I-378 | A |
| I-379 | B |
| I-380 | B |
| I-382 | A |
| I-383 | A |
| I-384 | A |
| I-385 | B |
| I-386 | B |
| I-387 | B |
| I-388 | B |
| I-389 | B |
| I-390 | B |
| I-391 | B |
| I-392 | B |
| I-393 | D |
| I-394 | A |
| I-395 | A |
| I-396 | B |
| I-397 | A |
| I-398 | A |
| I-399 | B |
| I-400 | A |
| I-401 | A |
| I-402 | E |
| I-403 | E |
| I-404 | E |
| I-405 | B |
| I-406 | B |
| I-407 | A |
| I-408 | E |
| I-409 | B |
| I-410 | E |
| I-411 | B |
| I-412 | A |
| I-413 | A |
| I-414 | B |
| I-415 | B |
| I-416 | A |
| I-417 | A |
| I-418 | B |
| I-419 | A |
| I-420 | A |
| I-421 | C |
| I-422 | A |
| I-423 | A |
| I-424 | A |
| I-426 | A |
| I-427 | B |
| I-428 | A |
| I-430 | D |
| I-431 | E |
| I-432 | D |
| I-433 | B |
| I-434 | C |
| I-435 | B |
| I-436 | C |
| I-437 | B |
| I-438 | B |
| I-439 | B |
| I-440 | B |
| I-441 | B |
| I-442 | C |
| I-443 | B |
| I-444 | B |
| I-445 | A |
| I-446 | B |
| I-447 | A |
| I-448 | E |
| I-449 | E |
| I-450 | E |
| I-451 | C |
| I-452 | B |
| I-453 | D |
| I-454 | E |
| I-455 | E |
| I-456 | E |
| I-457 | D |
| I-458 | E |
| I-459 | E |
| I-460 | E |
| I-461 | B |
| I-462 | D |
| I-463 | B |
| I-464 | B |
| I-465 | A |
| I-466 | C |
| I-467 | B |
| I-468 | B |
| I-469 | D |
| I-470 | B |
| I-471 | E |
| I-472 | C |
| I-473 | D |
| I-474 | E |
| I-475 | A |
| I-476 | E |
| I-477 | D |
| I-478 | B |
| I-479 | E |
| I-480 | E |
| I-481 | E |
| I-482 | E |
| I-483 | E |
| I-484 | E |
| I-485 | B |
| I-486 | D |
| I-488 | D |
| I-489 | E |
| I-490 | E |
| I-491 | C |
| I-492 | B |
| I-493 | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I: 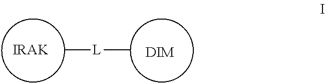
or a pharmaceutically acceptable salt thereof, wherein:
IRAK is an IRAK binding moiety capable of binding to IRAK4, said compound of formula I is a compound of formula I-a: 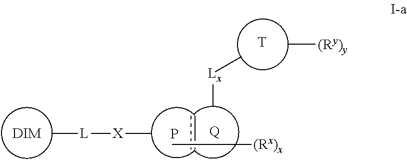
or a pharmaceutically acceptable salt thereof, wherein:
each R^{x} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —N⁺(O⁻)R₂, —OP(O)R₂, —OP(O)(OR)₂, —OP(O)(OR)NR₂, —OP(O)(NR₂)₂, —P(O)R₂, —SiR₃, —Si(OR)R₂, or 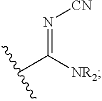
or
two R^{x} groups are optionally taken together to form an optionally substituted 5-6 membered partially unsaturated or aryl fused ring having 0-3 heteroatoms independently selected from nitrogen, oxygen, or sulfur, or a 3-5 membered saturated or partially unsaturated carbocyclic or heterocyclic spiro fused ring having 0-3 heteroatoms independently selected from nitrogen, oxygen, or sulfur,
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form a 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur;
each R^{y} is independently hydrogen, deuterium, R^{z}, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(S)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —SiR₃, —SF₅, or 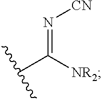
or
a single R^{y} and a single R^{x} are optionally taken together with their intervening atoms to form a 8-20 membered saturated or partially unsaturated carbocyclic or heterocyclic monocyclic or bicyclic ring having 1-10 heteroatoms, independently selected from nitrogen, oxygen, and sulfur;
each R^{z} is independently an optionally substituted group selected from C₁₋₆ aliphatic, phenyl, a 4-9 membered saturated or partially unsaturated carbocyclic or heterocyclic monocyclic, bicyclic, bridged bicyclic, or spirocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring P and Ring Q are fused rings independently selected from benzo, a 4-7 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring P and Ring Q are independently and optionally substituted with 1-2 oxo groups;
Ring T is selected from phenyl, a 4-7 membered saturated or partially unsaturated carbocyclic or heterocyclic ring having 1-3 heteroatoms independently selected from nitrogen, oxygen, and sulfur, or a 5-9 membered mono- or bicyclic heteroaryl ring having 1-4 heteroatoms independently selected from nitrogen, oxygen, and sulfur, wherein Ring T is further optionally substituted with 1-2 oxo groups;
L^{x} is a covalent bond or a C₁₋₃ bivalent straight or branched saturated or unsaturated hydrocarbon chain wherein 1-2 methylene units of the chain are independently and optionally replaced with -Cy^{x}-, —O—, —S—, —C(O)—, —C(S)—, —CR₂—, —CRF—, —CF₂—, —NR—, —N═CR—, —CR═CR—, or —S(O)₂—, wherein R of —CR₂—CRF—, —NR—, —N═CR—, or —CR═CR— can combine with R^{x} or R^{y} to form a 4-7 membered saturated or partially unsaturated carbocyclic or heterocyclic ring having 1-3 heteroatoms independently selected from nitrogen, oxygen, and sulfur;
-Cy^{x}- is an optionally substituted ring selected from a 3-5 membered saturated or partially unsaturated carbocyclic or heterocyclic ring having 0-3 heteroatoms independently selected from nitrogen, oxygen, and sulfur, or a 5 membered heteroaryl ring having 1-4 heteroatoms independently selected from nitrogen, oxygen, and sulfur, wherein -Cy^{x}- is optionally substituted with 1-2 oxo groups;
X is a covalent bond or a 4-6 membered saturated or partially unsaturated carbocyclic or heterocyclic ring having 1-2 heteroatoms independently selected from nitrogen, oxygen, and sulfur; [symbol]
is a single or double bond;
x is 0, 1, 2, 3 or 4;
y is 0, 1, 2, 3 or 4;
L is a covalent bond or a bivalent, saturated or unsaturated, straight or branched C₁₋₅₀ hydrocarbon chain, wherein 0-6 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 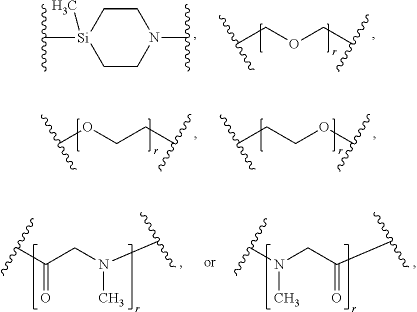
wherein
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur, and
r is 0, 1, 2, 3, 4, 5, 6, 7, 8, 9, or 10; and
DIM is a degradation inducing moiety.

2. The compound of claim 1, wherein said compound is any one of the following formulae: 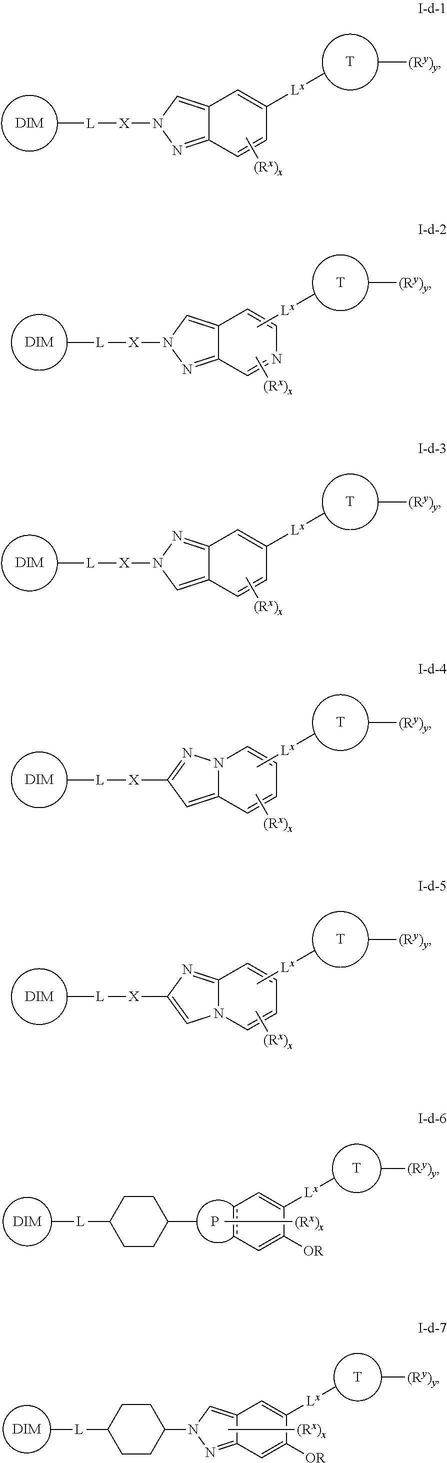
or a pharmaceutically acceptable salt thereof.

3. The compound of claim 1 or claim 2, wherein the DIM is a ligase binding moiety (LBM), lysine mimetic, or hydrogen atom.

4. The compound of claim 3, wherein LBM is a cereblon E3 ubiquitin ligase binding moiety and said compound is of formula I-aa: 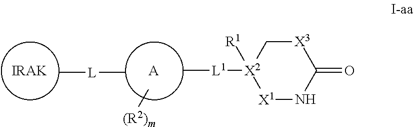
or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 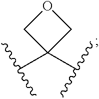 [SMILES: *C1(*)COC1]
X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from 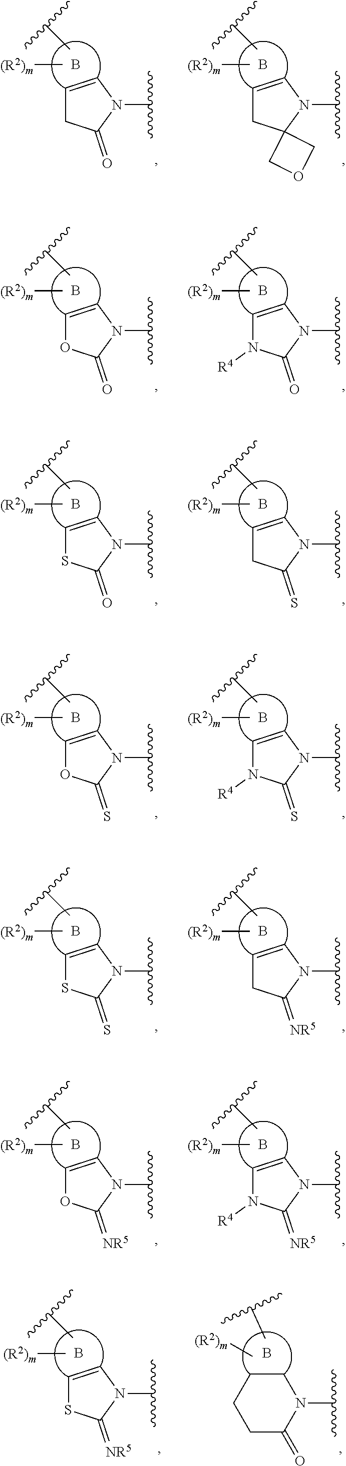 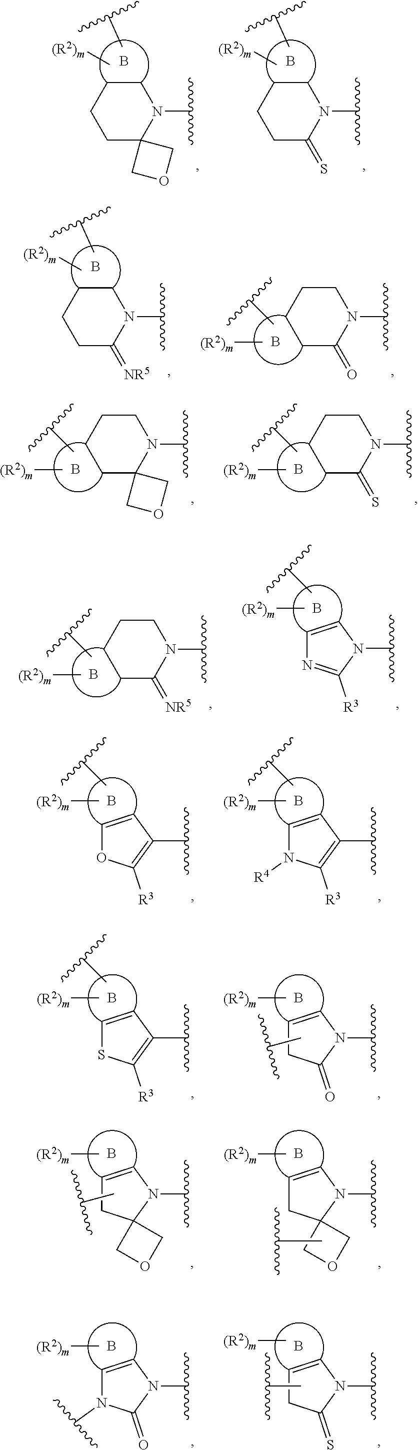 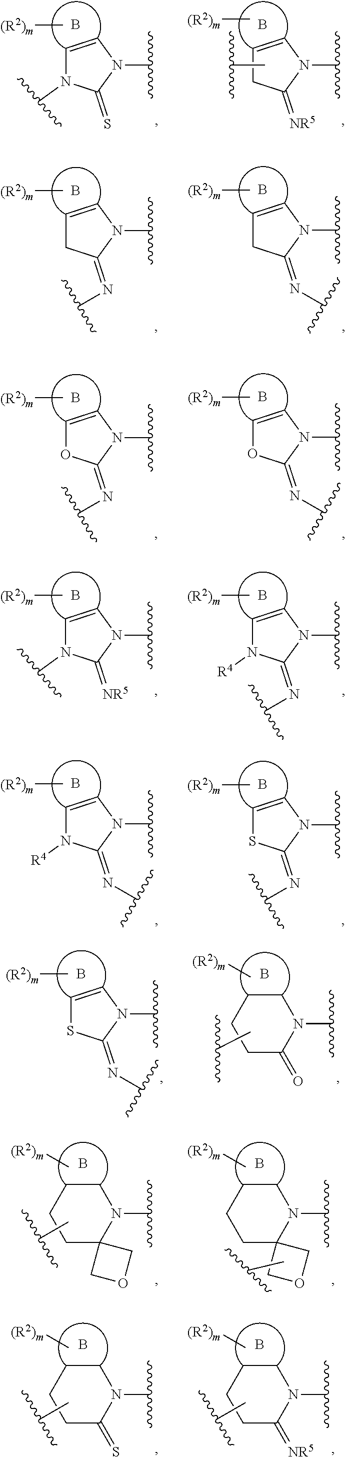 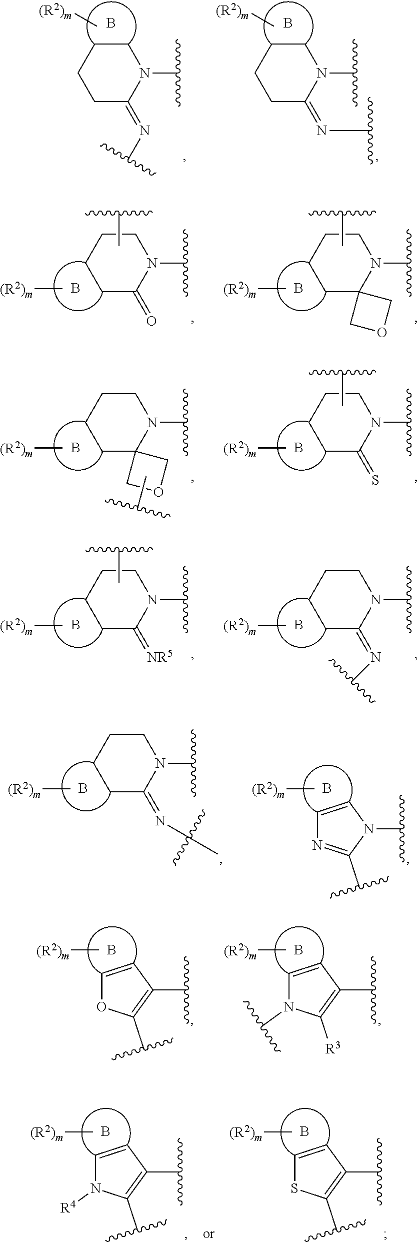
Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

5. The compound of claim 4, wherein said compound is any one of the following formulae: 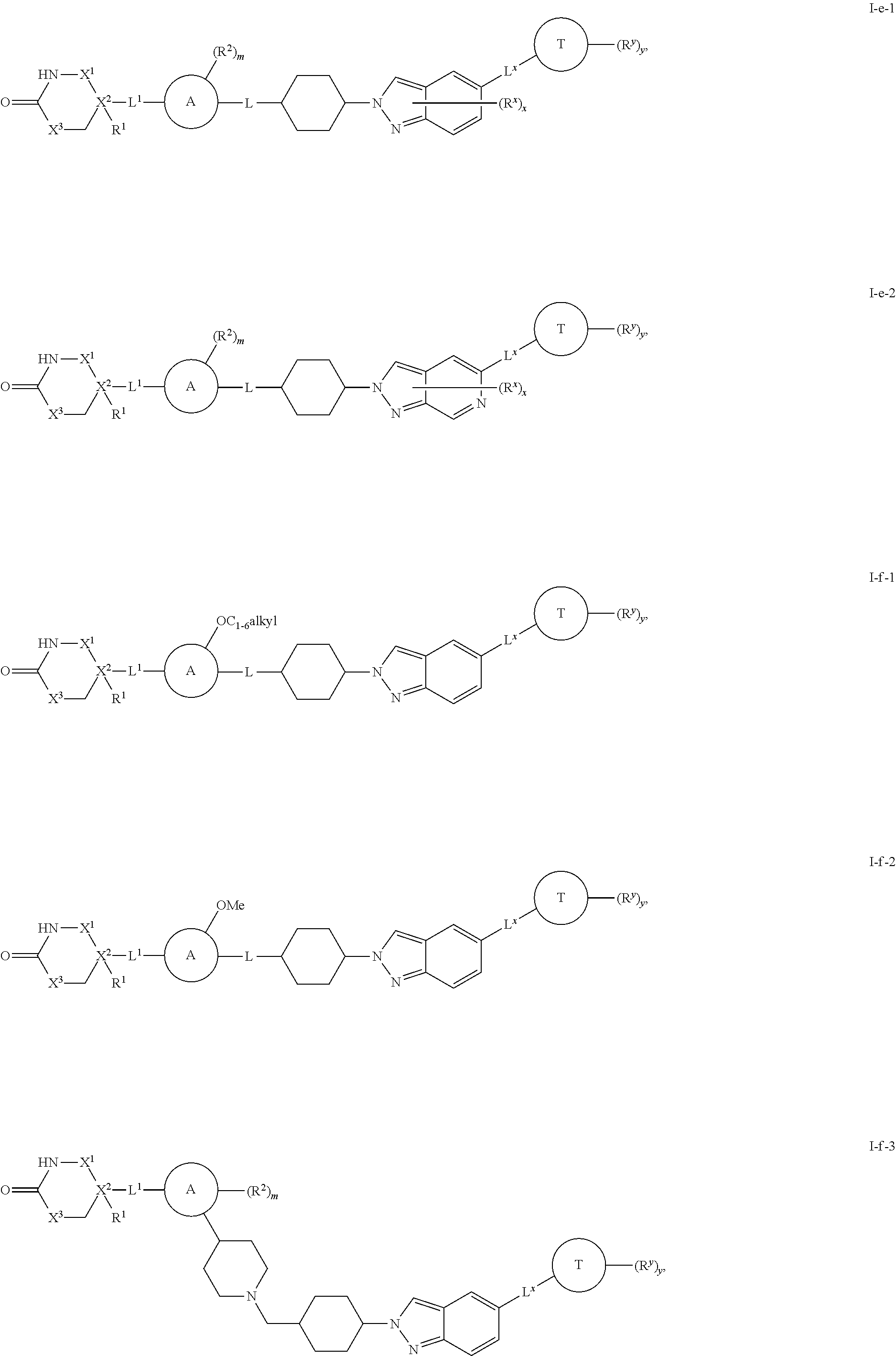 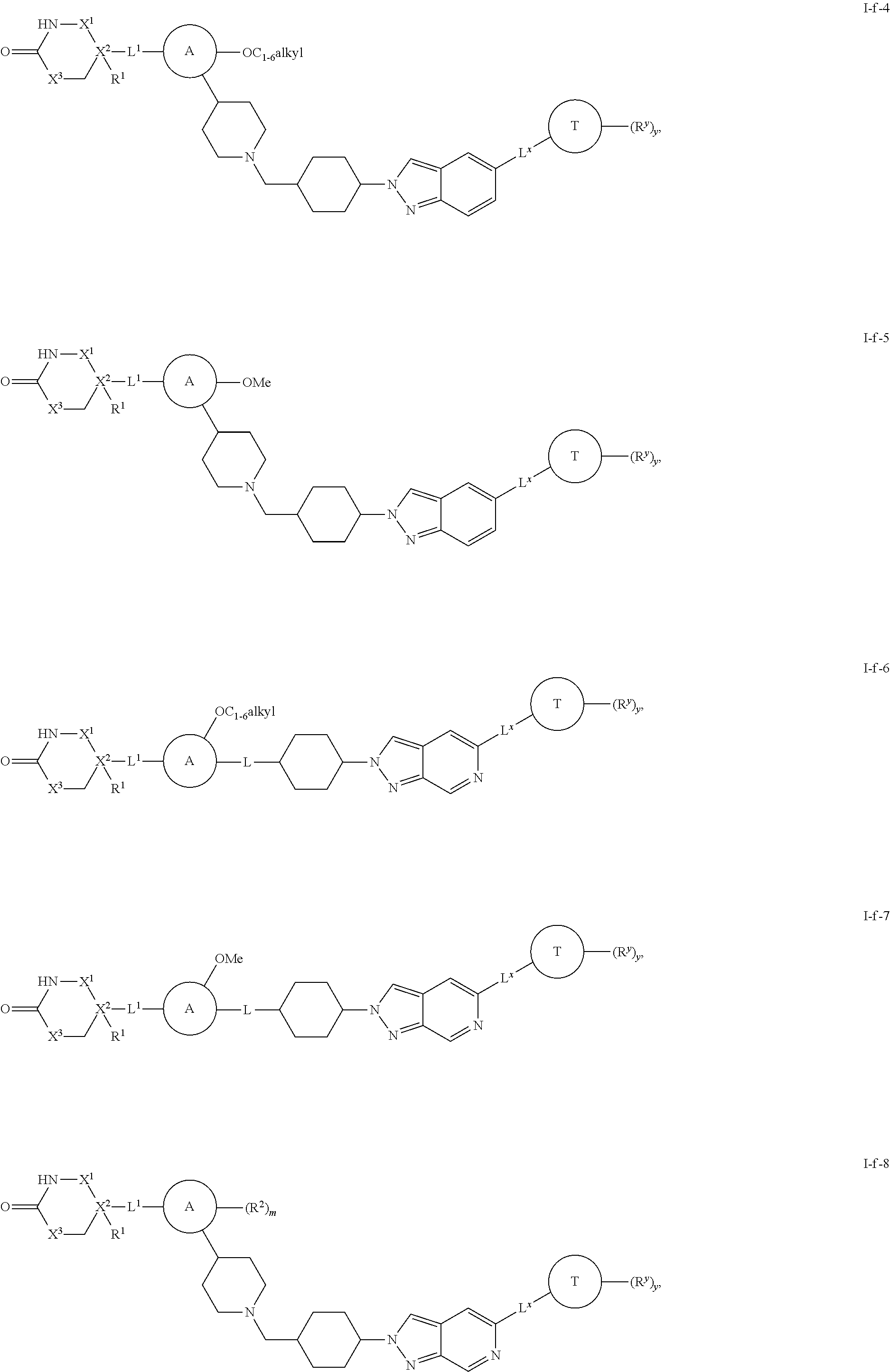 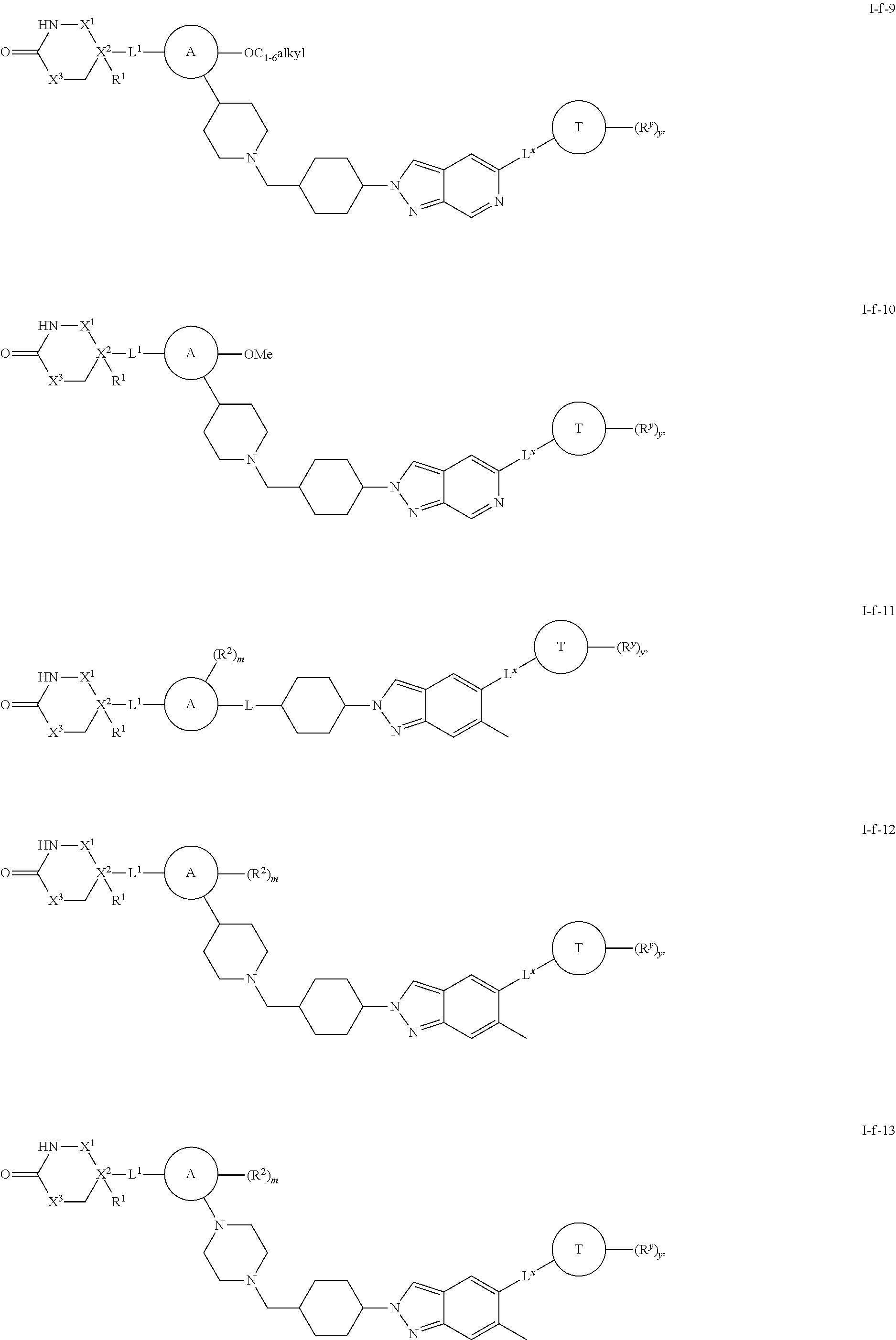 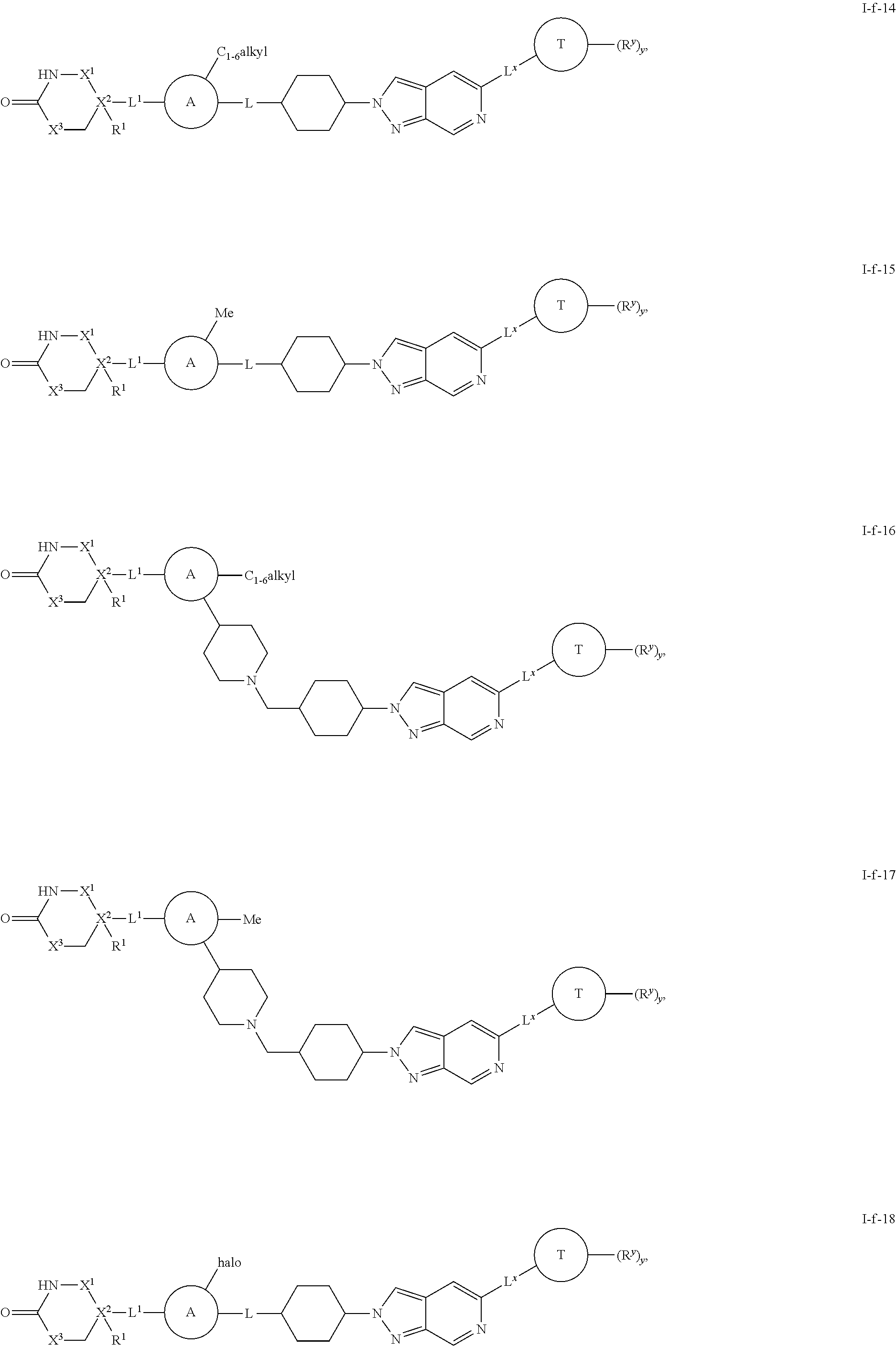 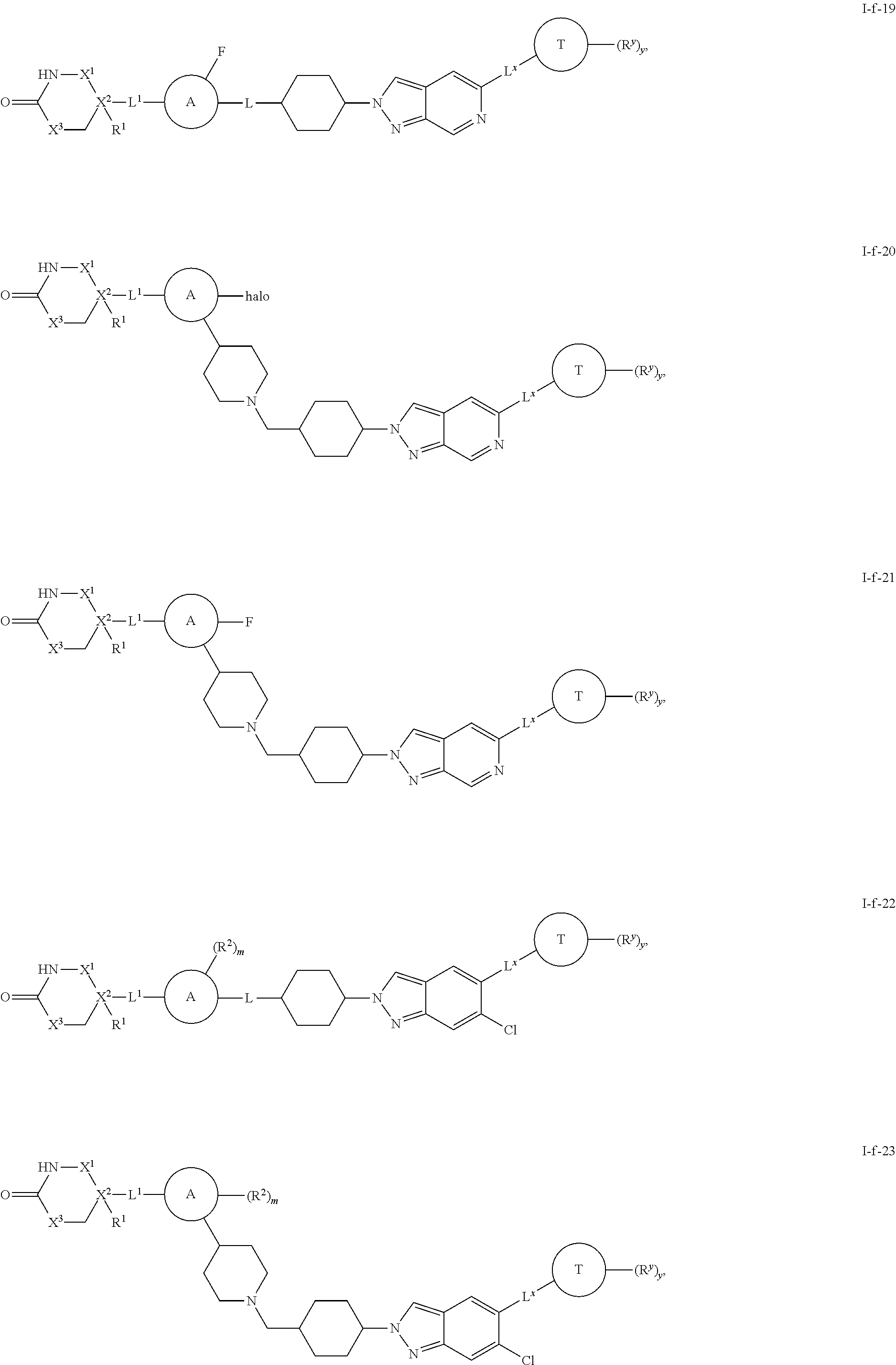 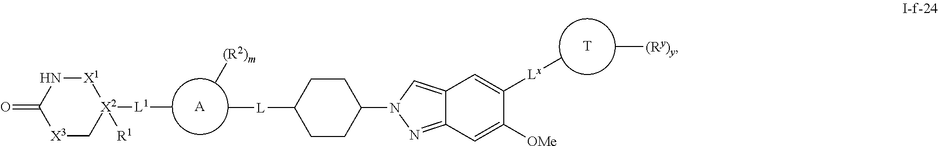 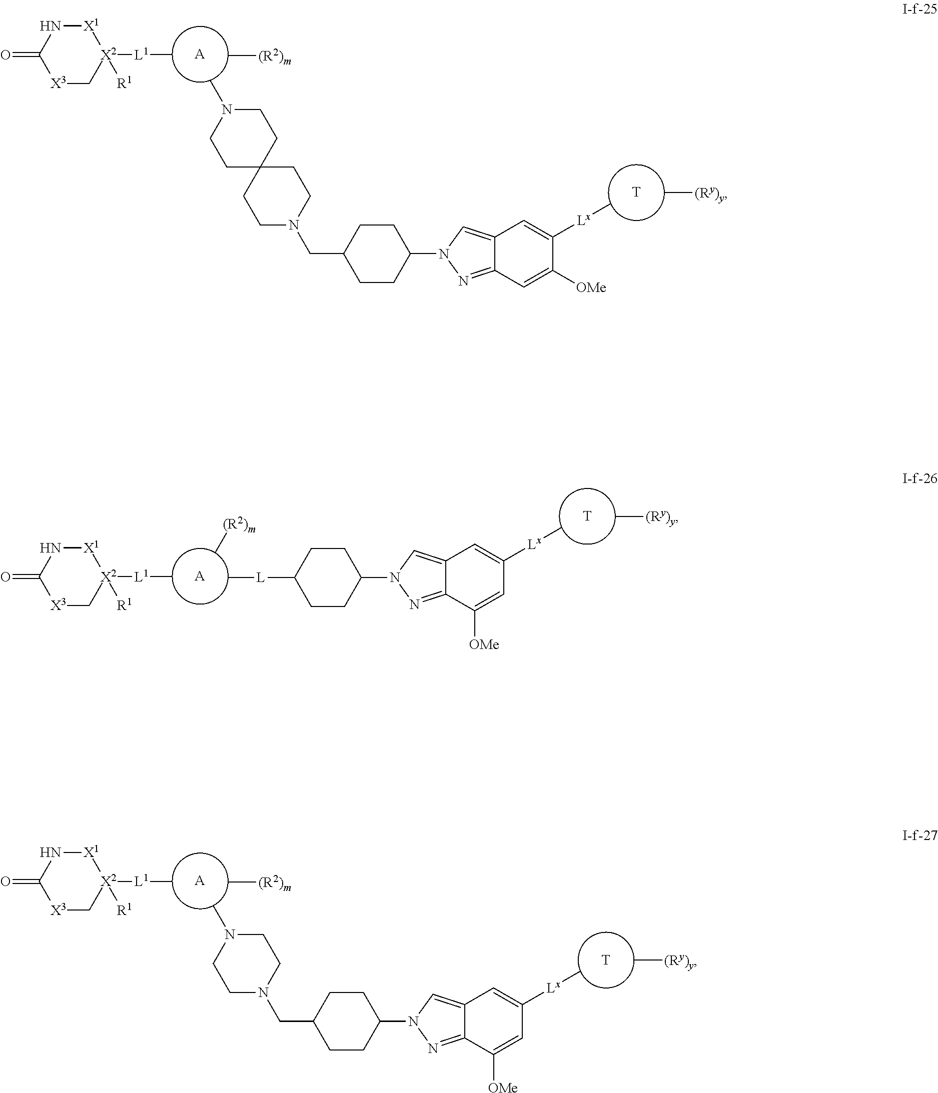 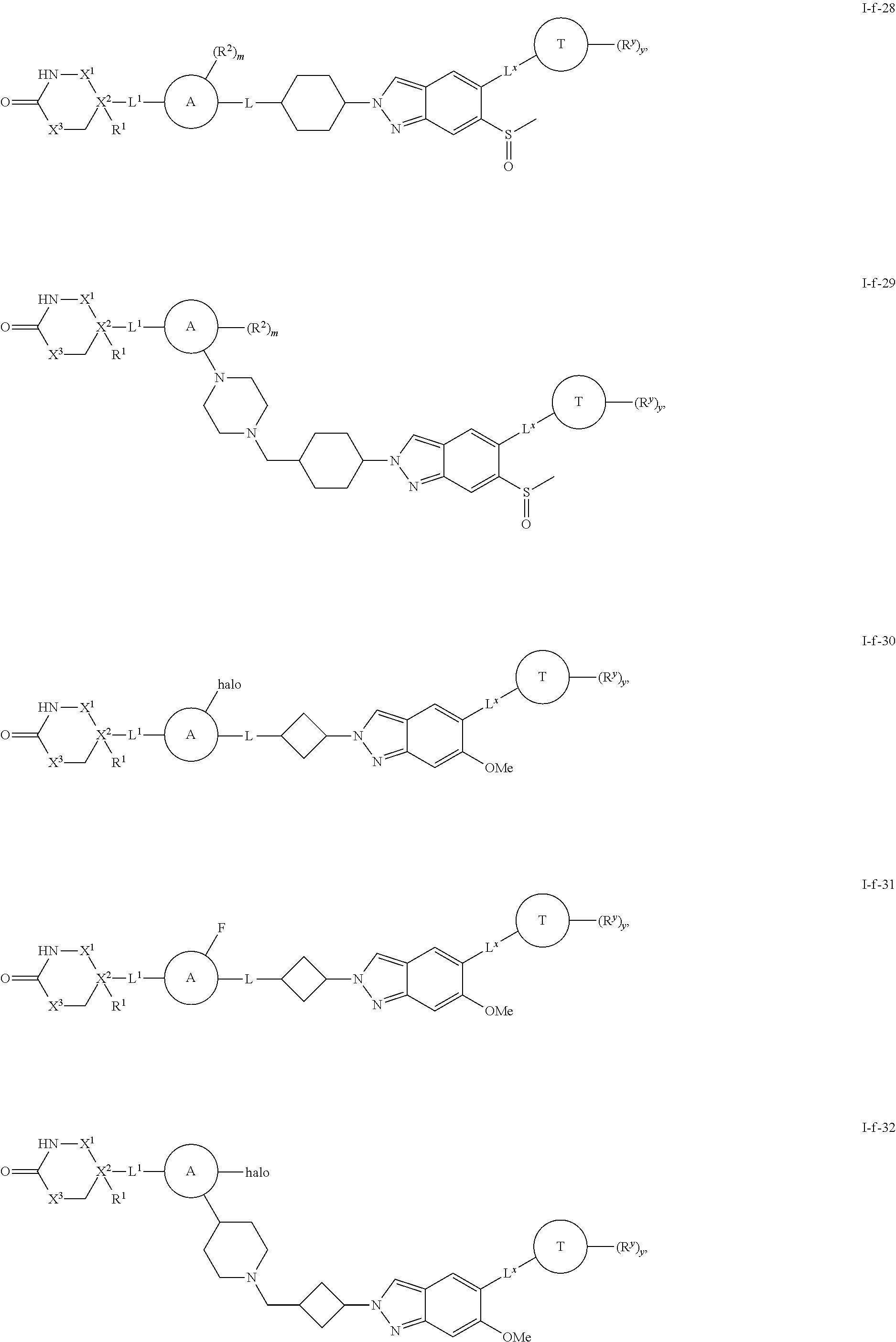 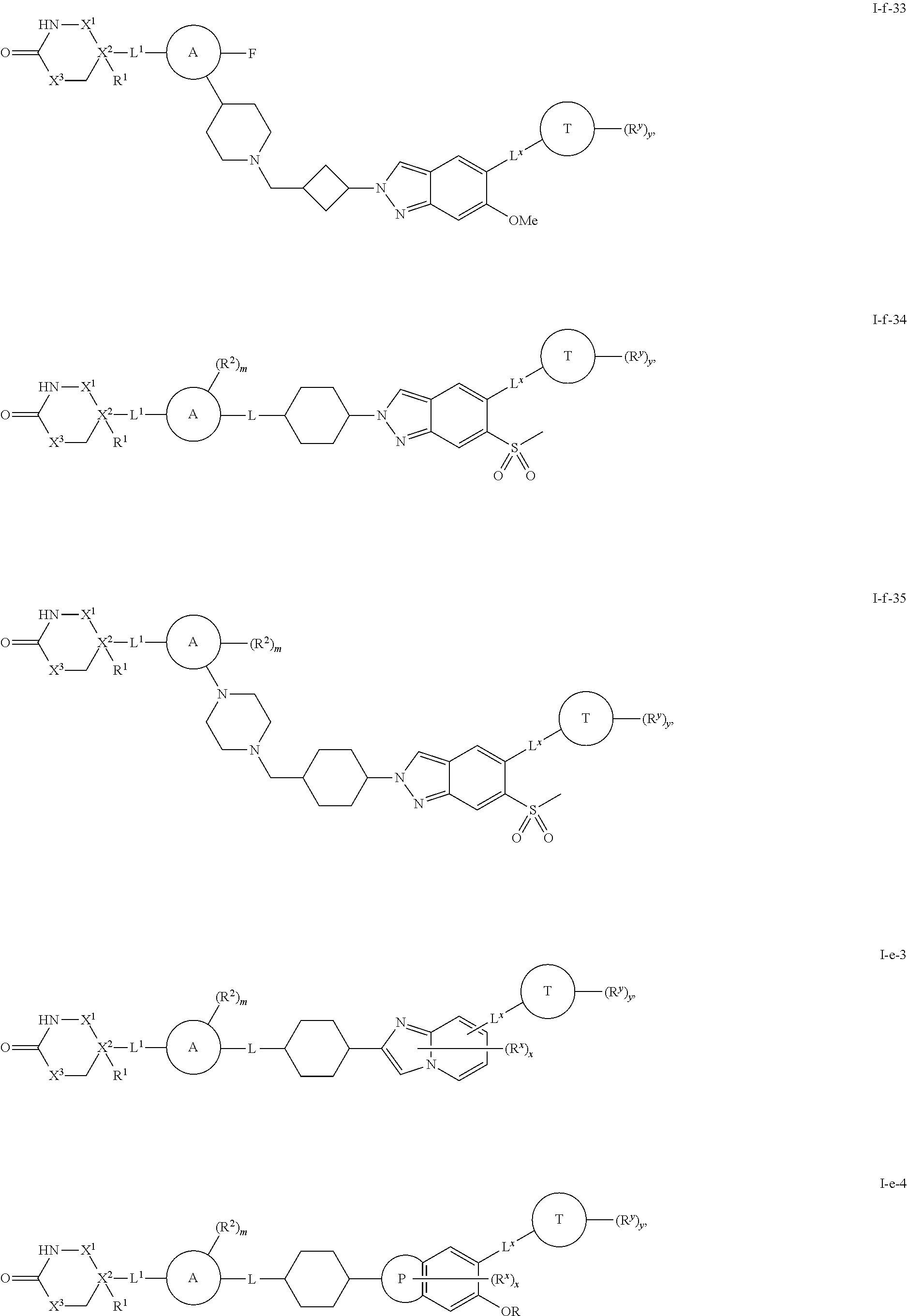 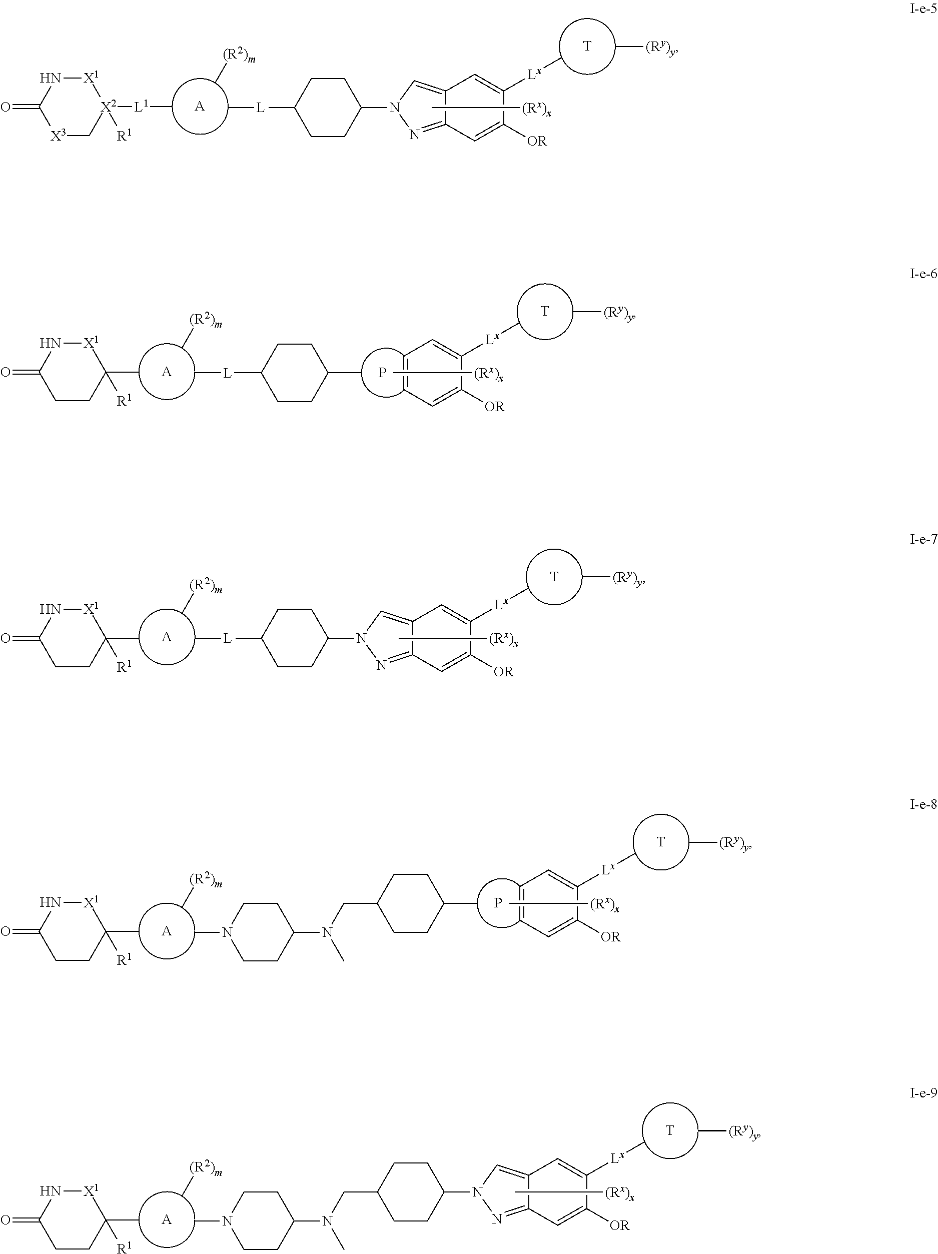 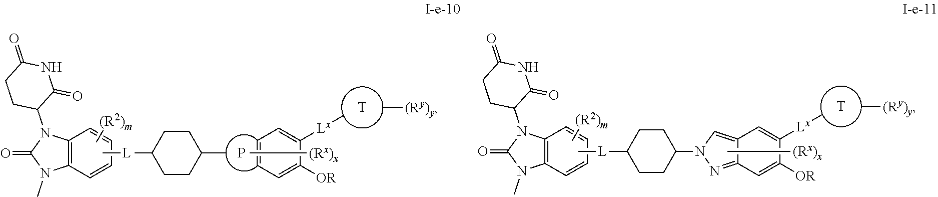
or a pharmaceutically acceptable salt thereof.

6. The compound of claim 3, wherein LBM is a cereblon E3 ubiquitin ligase binding moiety and said compound is of formula I-nn: 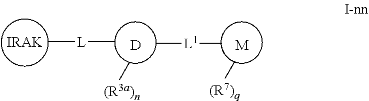
or a pharmaceutically acceptable salt thereof, wherein:
Ring E is selected from 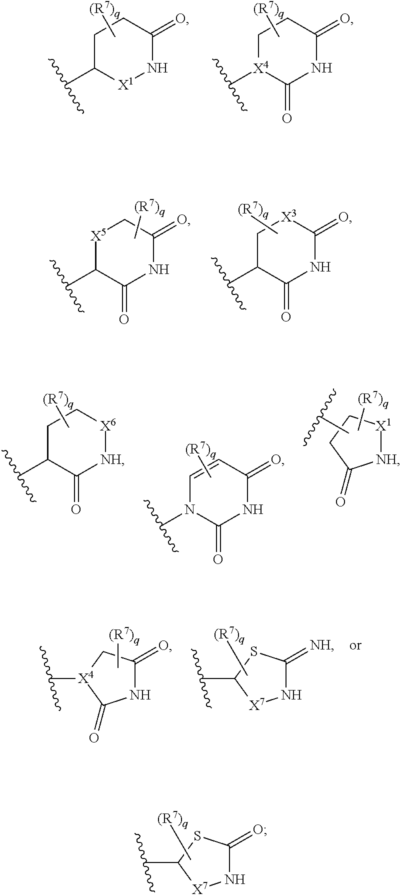
each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 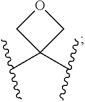 [SMILES: *C1(*)COC1]
each of X³ and X⁵ is independently a bivalent moiety selected from a covalent bond, —CR₂—, —NR—, —O—, —S—, or —SiR₂—;
X⁴ is a trivalent moiety selected from 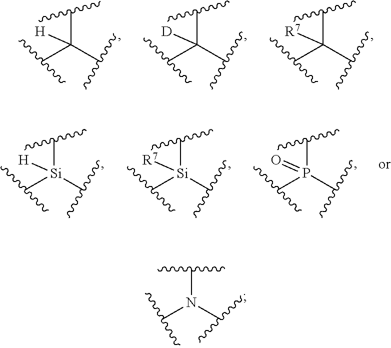
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH—)₂R, —SiR₃, or an optionally substituted C₁₋₄ aliphatic; or
R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur;
two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur;
two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
n is 0, 1, 2, 3, or 4; and
q is 0, 1, 2, 3, or 4.

7. The compound of claim 6, wherein said compound is any one of the following formulae: 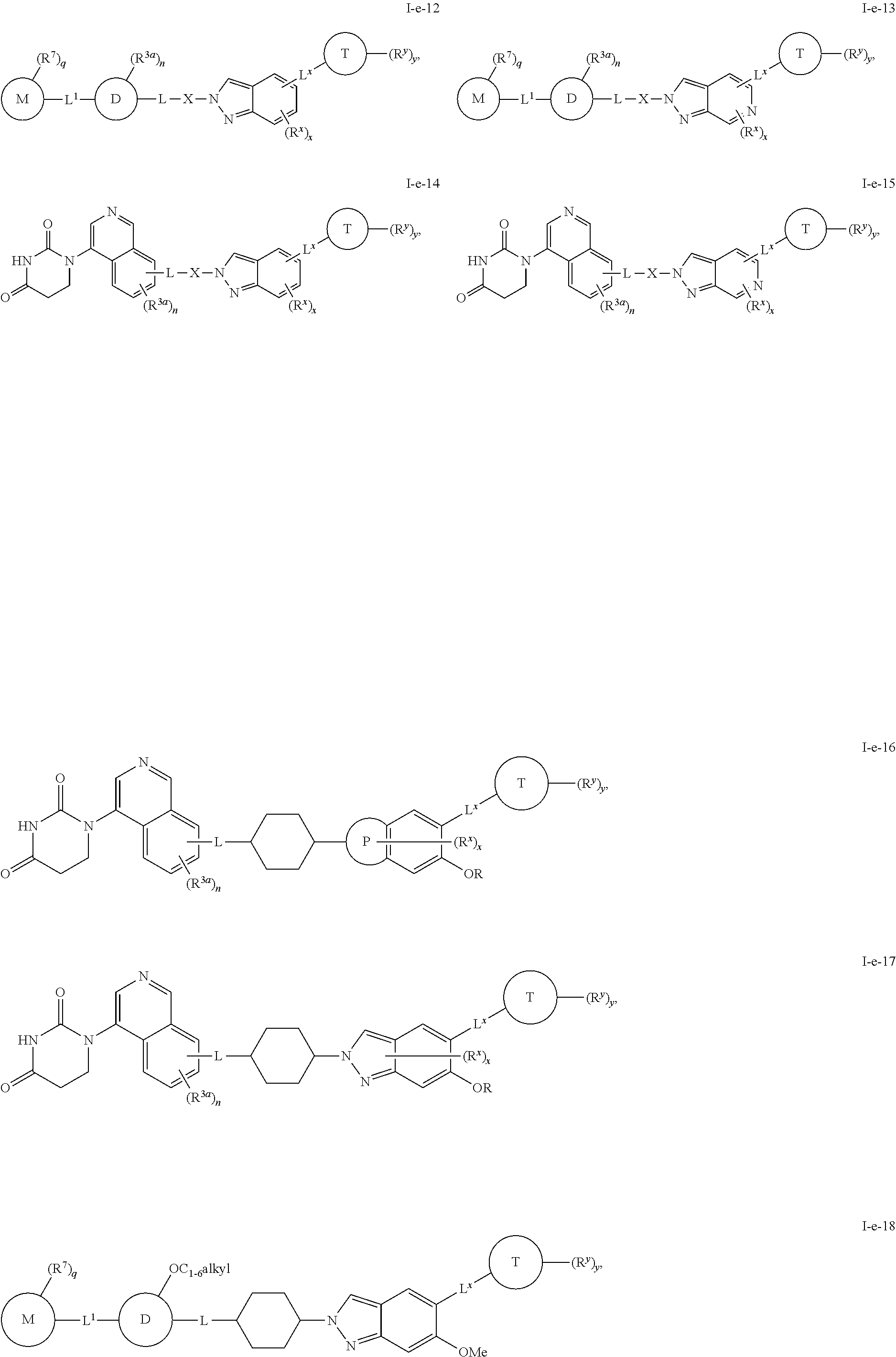 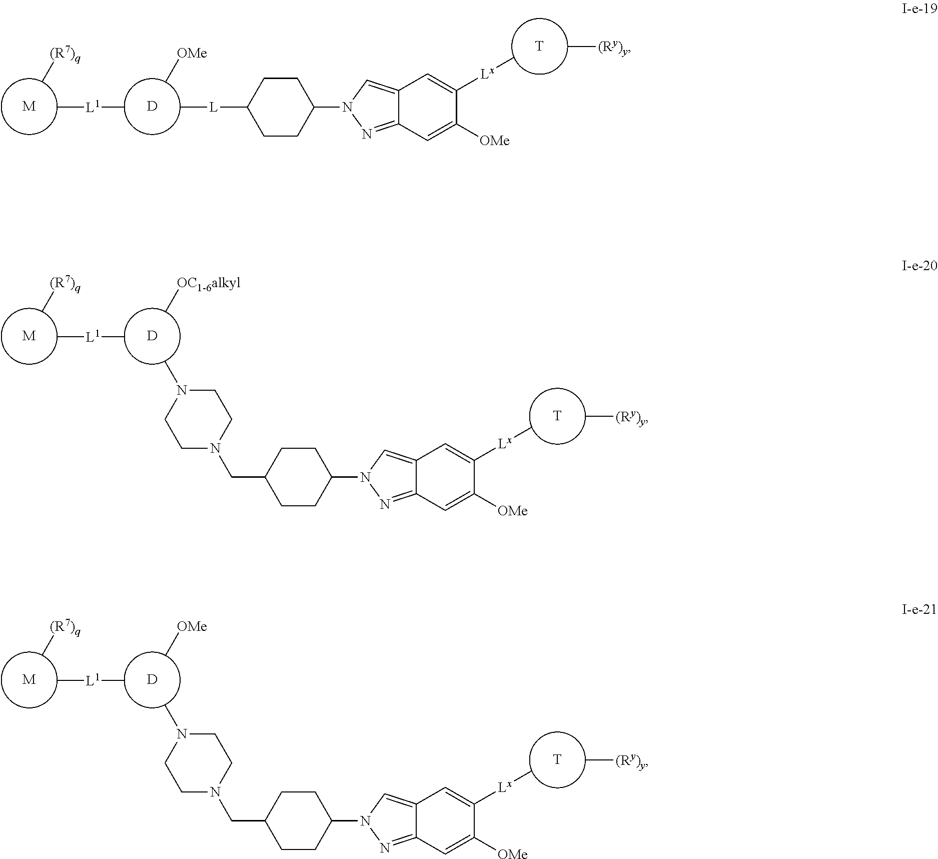
or a pharmaceutically acceptable salt thereof.

8. The compound of claim 3, wherein LBM is a cereblon E3 ubiquitin ligase binding moiety and said compound is selected from any one of the following formulae: 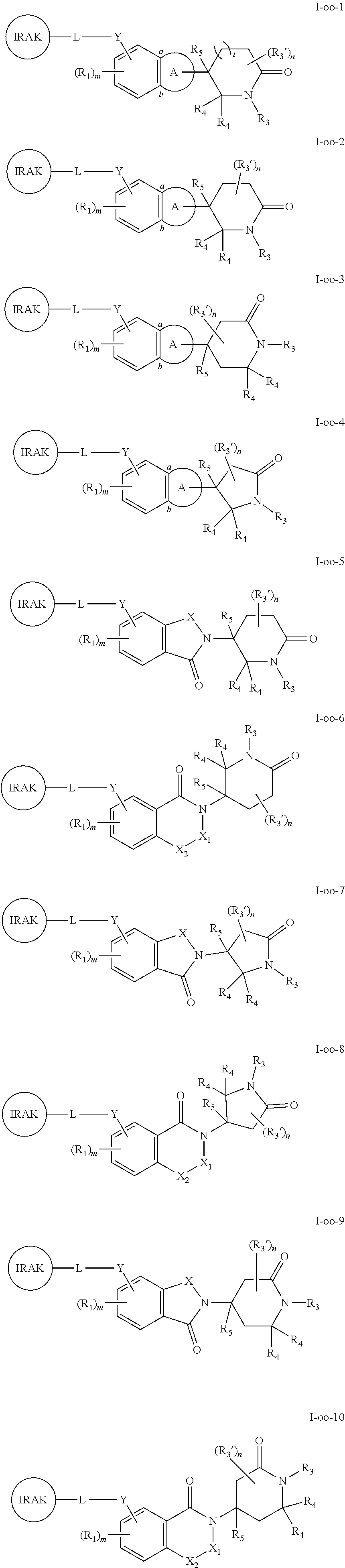
or a pharmaceutically acceptable salt thereof, and wherein: 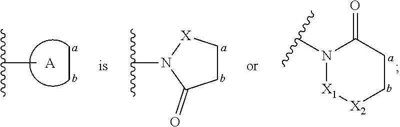
Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁-X₂ is C(R₃)═N or C(R₃)₂—C(R₃)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C1-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆alkyl, C₁-C₆haloalkyl, C₁-C₆alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and 0;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2; 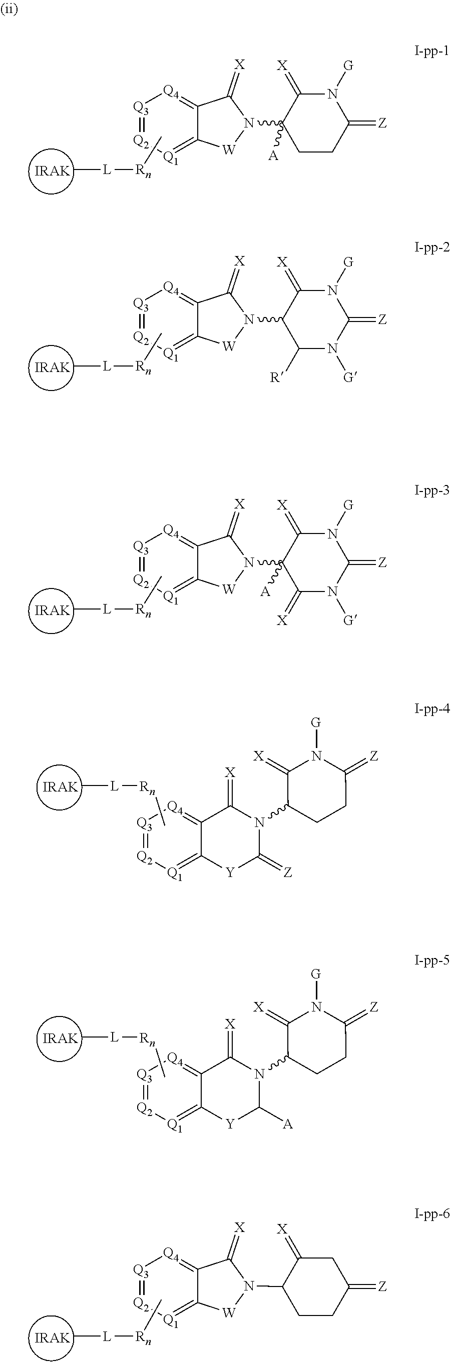
or a pharmaceutically acceptable salt thereof, and wherein each of the variables A, G, G′, Q₁, Q₂, Q₃, Q₄, R, R′, W, X, Y, Z, [symbol] and n is as defined and described in WO 2016/197114 and US 2018/0147202; 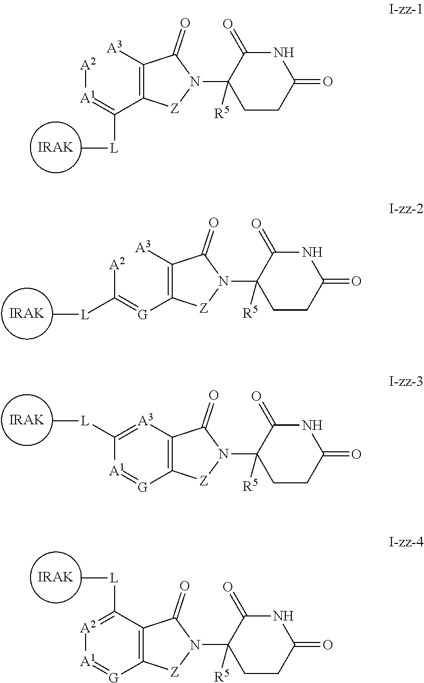
or a pharmaceutically acceptable salt thereof, and wherein each of the variables A¹, A², A³, R⁵, G and Z is as defined and described in WO 2017/176958; 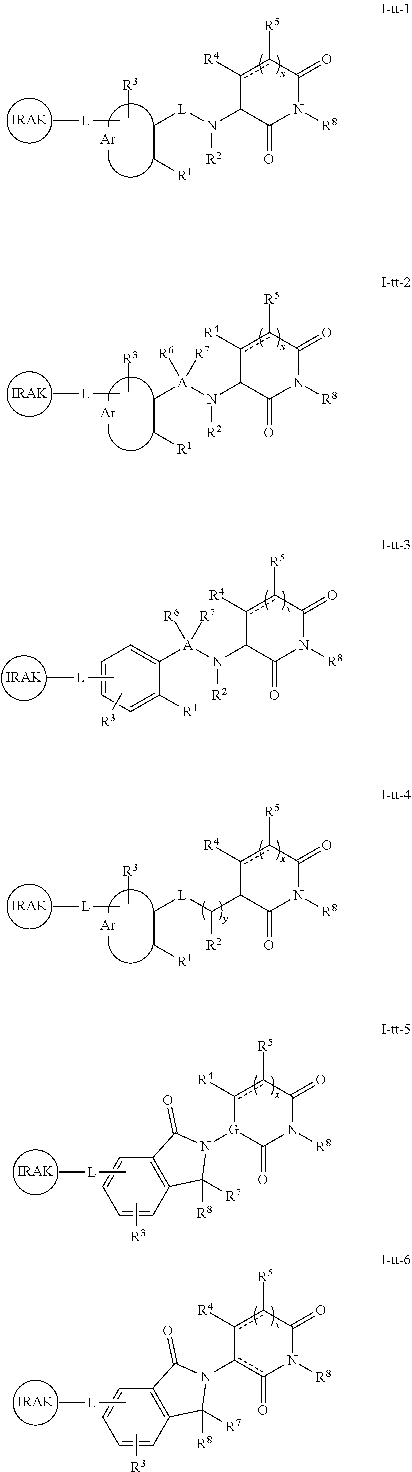 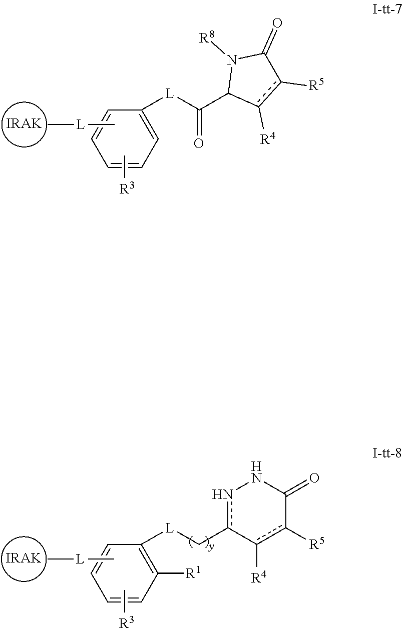
or a pharmaceutically acceptable salt thereof, and wherein each of the variables Ar, R¹, R², R³, R⁴, R⁵, R⁶, R⁷, R⁸, A, L, x, y, and [symbol] is as described and defined in WO 2017/161119; 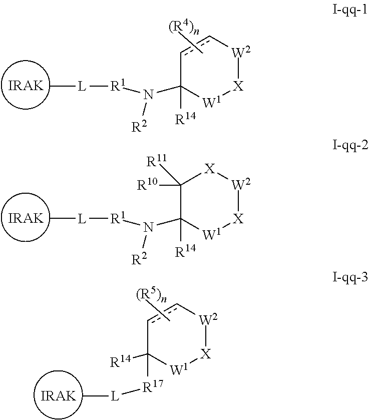
or a pharmaceutically acceptable salt thereof, wherein each of the variables R¹, R², R⁴, R⁵, R¹⁰, R¹¹, R¹⁴,
R¹⁷, W¹, W², X, [symbol] , and n is as defined in WO 2017/197051, and wherein 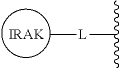
is attached to R¹, the ring formed b combining R¹ and R², or R¹⁷ at the site of attachment of R¹² as defined 2017/197051 such that 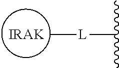
takes the place of the R¹² substituent; 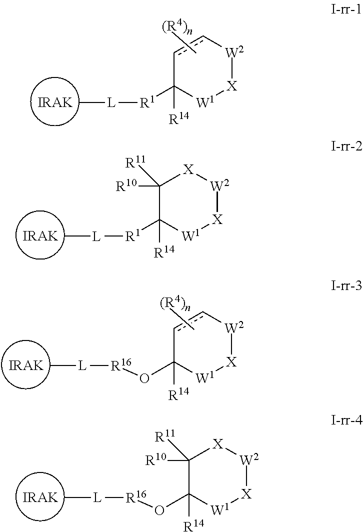
or a pharmaceutically acceptable salt thereof, wherein each of the variables R¹, R⁴, R¹⁰, R¹¹, R¹⁴, R¹⁶, W¹, W², X, [symbol] , and n is as defined in WO 2018/237026, and wherein 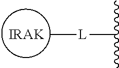
is attached to R¹ or R¹⁶ at the site of attachment of R¹² as defined in WO 2018/237026, such that 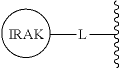
takes the place of the R¹² substituent; or 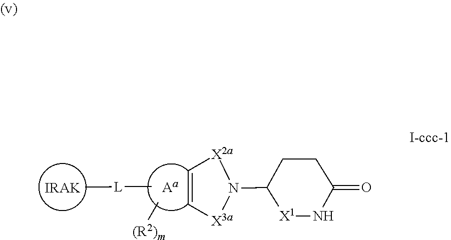
or a pharmaceutically acceptable salt thereof, wherein:
each of X¹, X^{2a}, and X^{3a} is independently a bivalent moiety selected from a covalent bond, —CH₂—, —C(O)—, —C(S)—, or 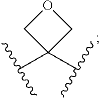 [SMILES: *C1(*)COC1]
R¹ is hydrogen, deuterium, halogen, —CN, —OR, —SR, —S(O)R, —S(O)₂R, —NR₂, or an optionally substituted C₁₋₄ aliphatic;
each of R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

9. The compound of claim 8, wherein said compound is any one of the following formulae: 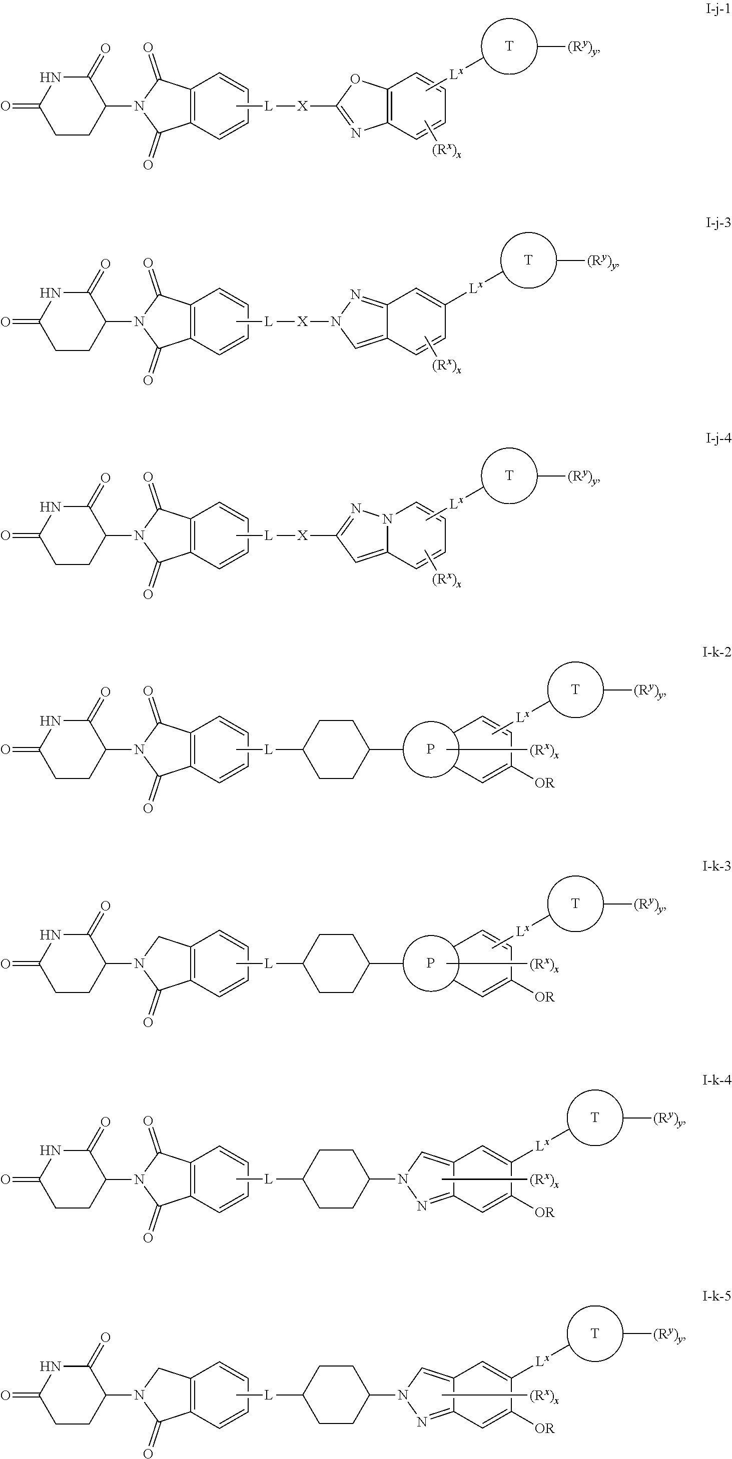 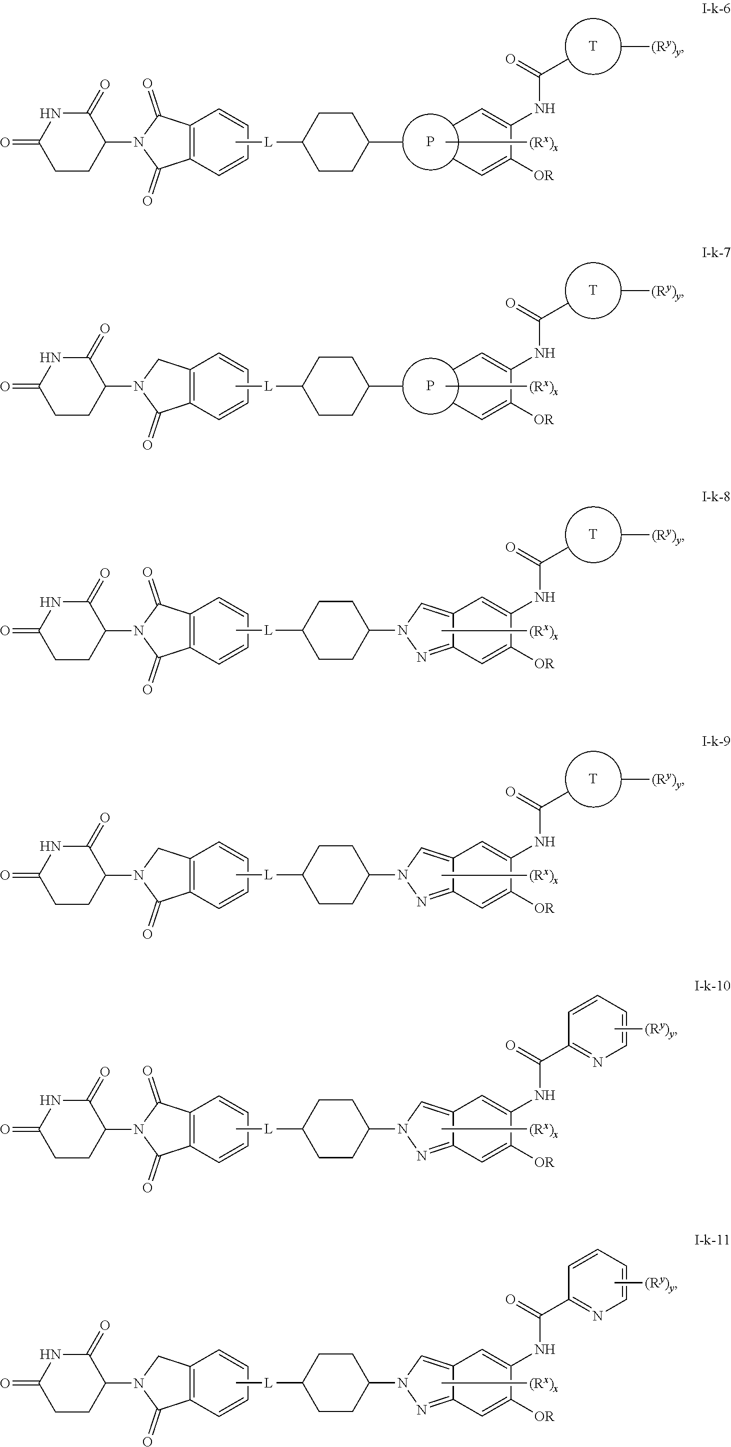 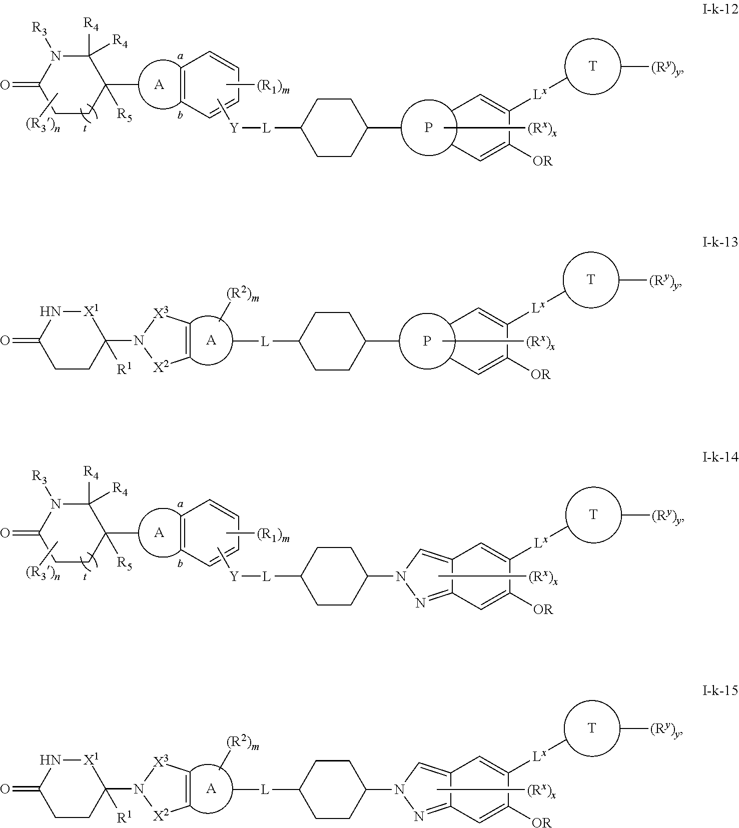
or a pharmaceutically acceptable salt thereof.

10. The compound of claim 3, wherein LBM is a VHL E3 ubiquitin ligase binding moiety and said compound is selected from any of the following formulae: 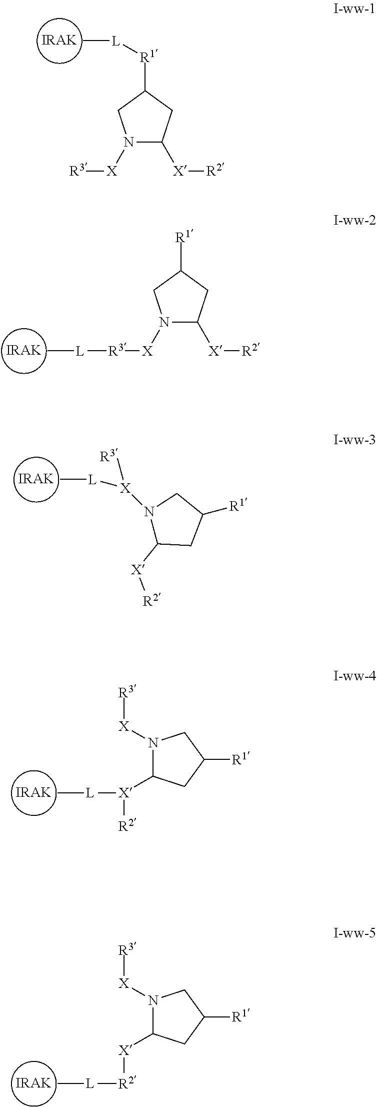
or a pharmaceutically acceptable salt thereof, and wherein each of the variables R^{1′}, R^{2′}, R^{3′}, X, and X′ is as defined and described in WO 2013/106643 and US 2014/0356322; 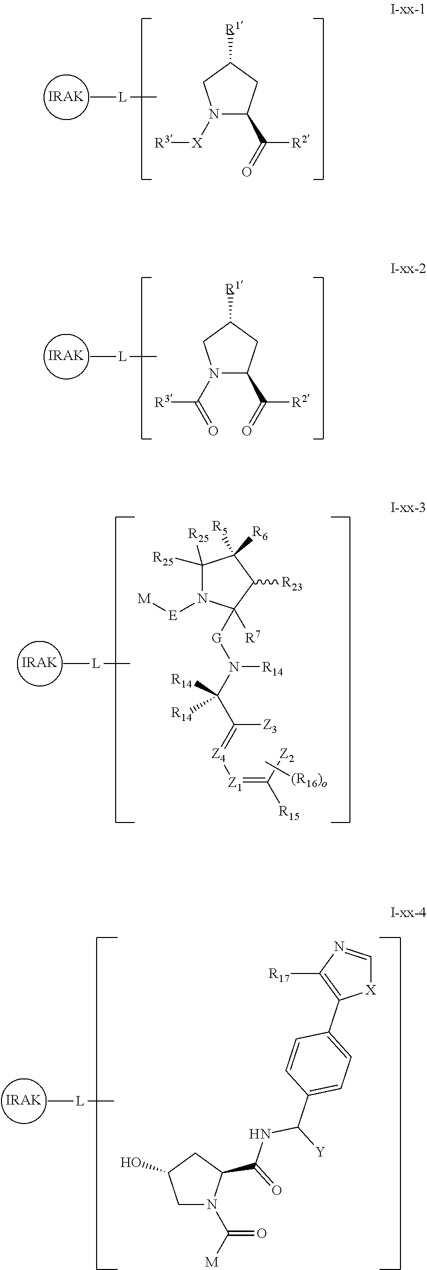 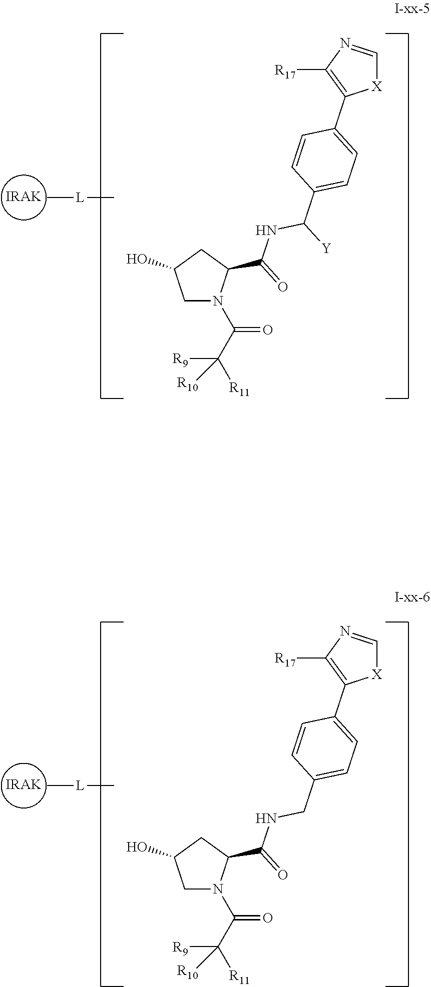
or a pharmaceutically acceptable salt thereof, and wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is as defined and described in WO 2016/149668 and US 2016/0272639; or 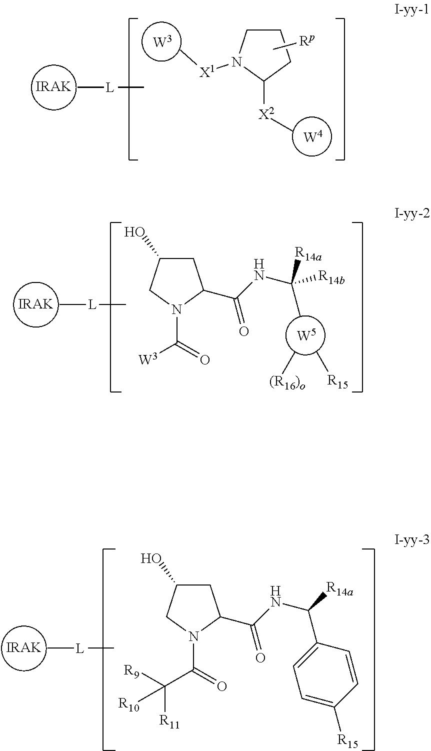
or a pharmaceutically acceptable salt thereof, and wherein each of the variables R^{P}, R₉, R₁₀, R₁₁, R₁₄ₐ, R_{14b}, R₁₅, R₁₆, W³, W⁴, W⁵, X¹, X², and o is as defined and described in WO 2016/118666 and US 2016/0214972.

11. The compound of claim 3, wherein LBM is a MDM2 E3 ubiquitin ligase binding moiety and said compound is selected from any of the following formulae: 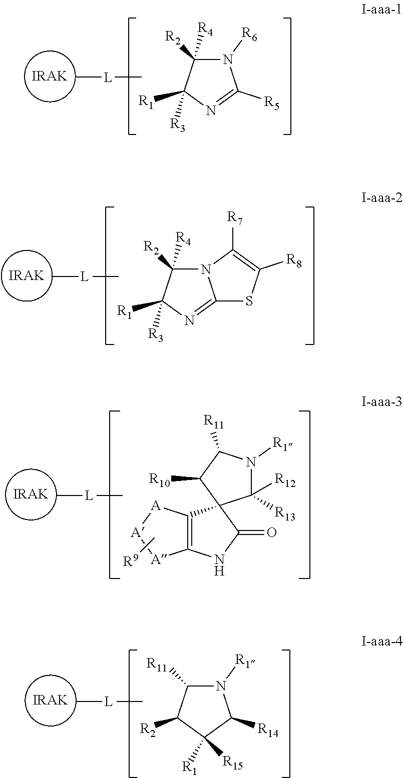 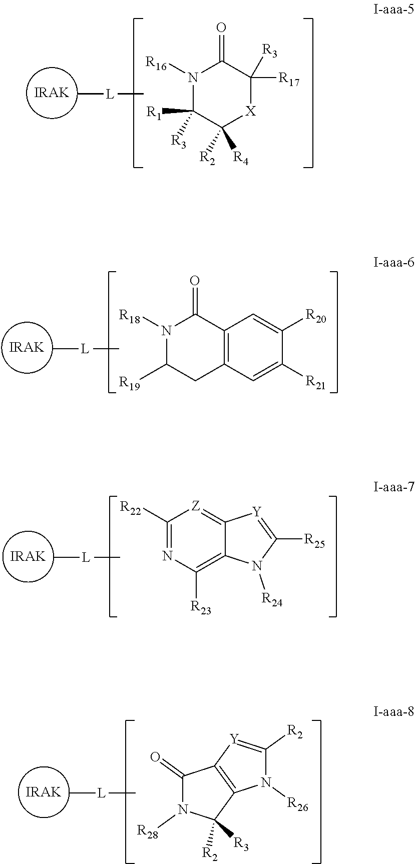 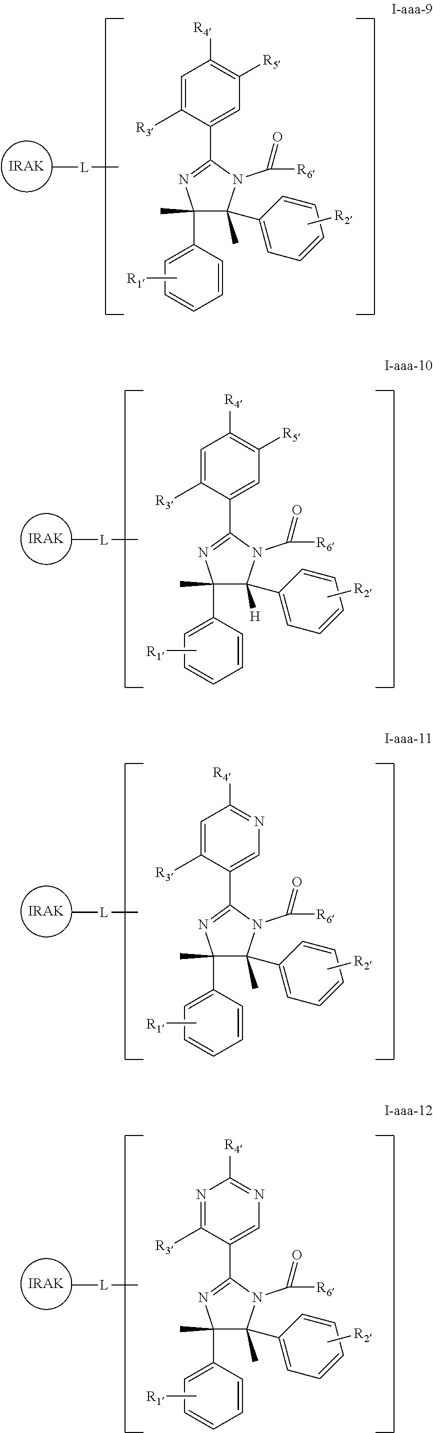 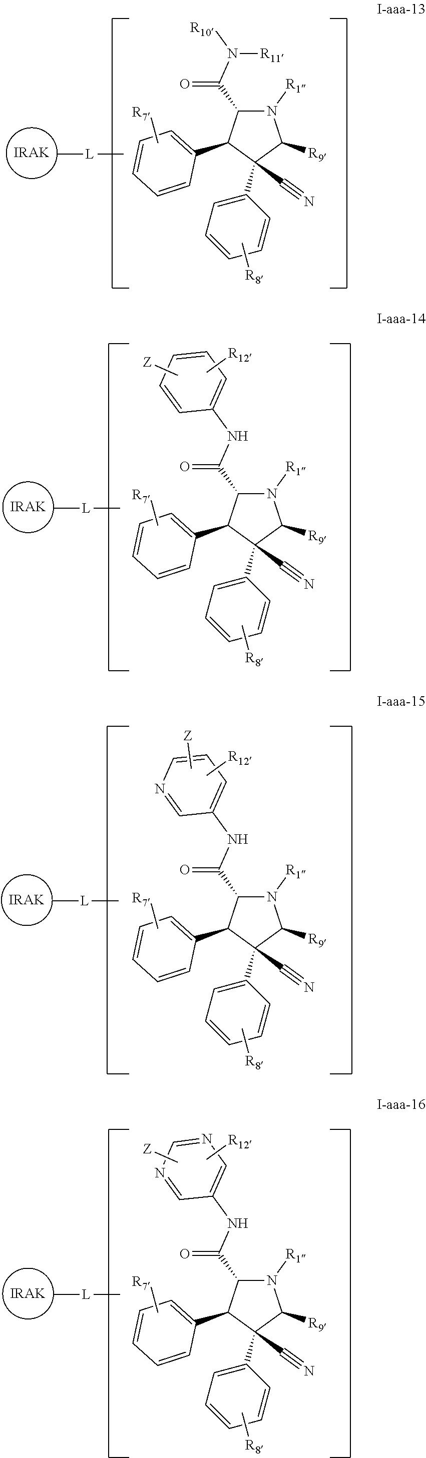 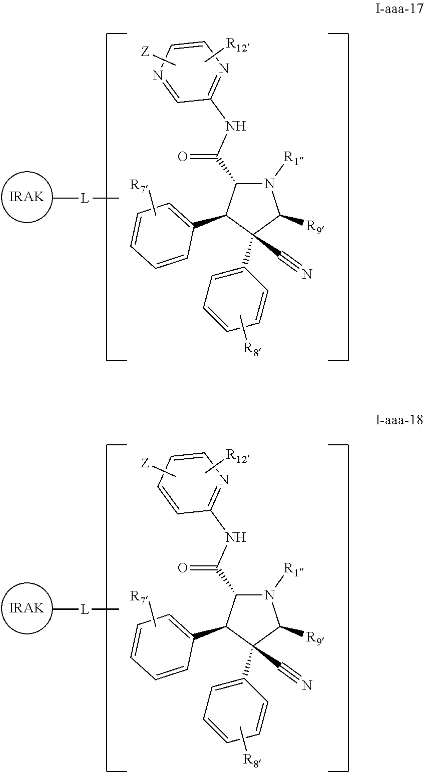
or a pharmaceutically acceptable salt thereof, and wherein each of the variables R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R_{1′}, R_{2′}, R_{3′}, R_{4′}, R_{5′}, R_{6′}, R_{7′}, R_{8′}, R_{9′}, R_{10′}, R_{11′}, R_{12′}, R_{1″}, A, A′, A″, X, Y, and Z is as defined and described in WO 2017/011371 and US 2017/0008904.

12. The compound of claim 3, wherein LBM is a IAP E3 ubiquitin ligase binding moiety and said compound is selected from any of the following formulae: 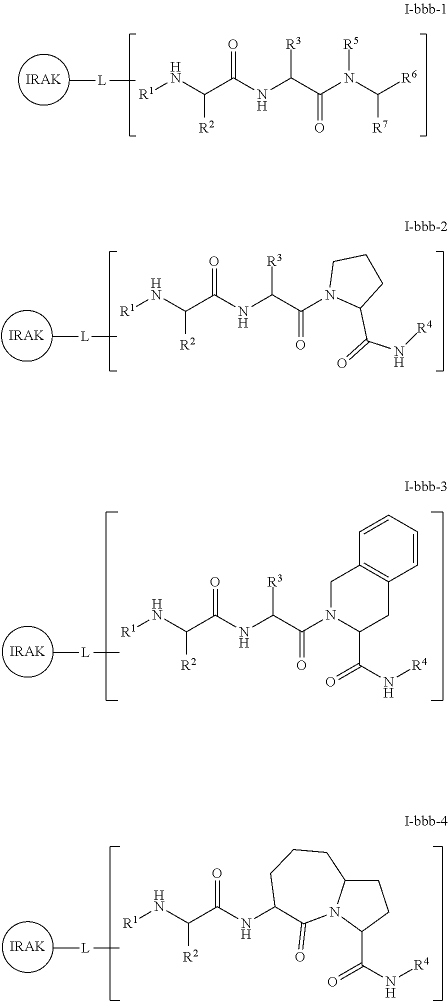
or a pharmaceutically acceptable salt thereof, and wherein each of the variables R¹, R², R³, R⁴, R⁵, R⁶, and R⁷, is as defined and described in WO 2017/011590 and US 2017/0037004.

13. The compound of claim 3, wherein DIM is a lysine mimetic and said compound is selected from any one of the following formulae: 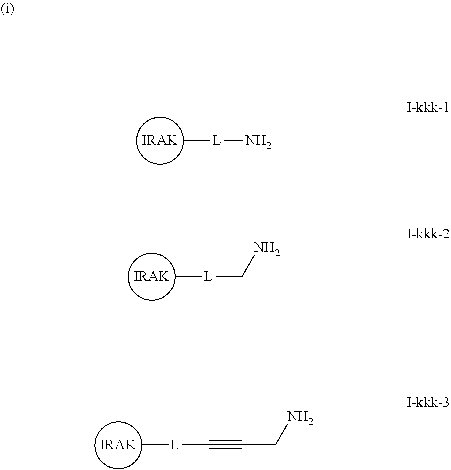
or a pharmaceutically acceptable salt thereof; and 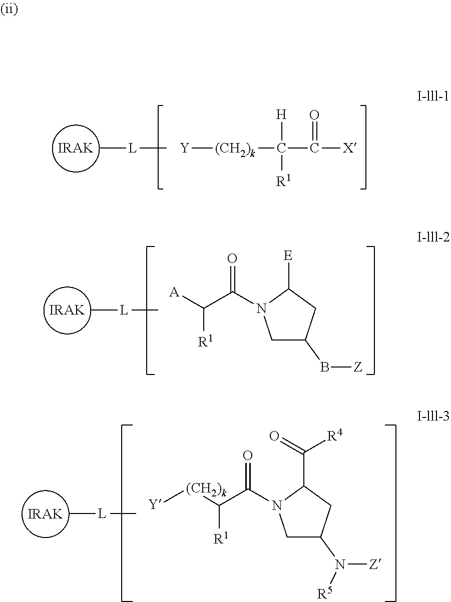
or a pharmaceutically acceptable salt thereof, wherein each of the variables R¹, R⁴, R⁵, A, B, E, Y, Y′, Z, Z′, and k are as defined and described in U.S. Pat. No. 7,622,496.

14. The compound of claim 3, wherein DIM is a hydrogen atom and said compound is formula I-mmm: 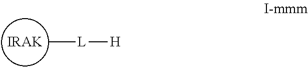
or a pharmaceutically acceptable salt thereof.

15. The compound according to any one of claims 1-14, wherein L is a bivalent, saturated or unsaturated, straight or branched C₁₋₃₀ hydrocarbon chain, wherein 0-6 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

16. The compound of any of one of claims 1-15, wherein said compound is selected from any one of the compounds depicted in Table 1, or a pharmaceutically acceptable salt thereof.

17. A pharmaceutical composition comprising a compound of any one of claims 1-16, and a pharmaceutically acceptable carrier, adjuvant, or vehicle.

18. A method of degrading and/or inhibiting an IRAK protein kinase in a patient or biological sample comprising administering to said patient, or contacting said biological sample with a compound of any one of claims 1-16, or a pharmaceutical composition thereof.

19. A method of treating an IRAK-mediated disorder, disease, or condition in a patient comprising administering to said patient a compound of any one of claims 1-16, or a pharmaceutical composition thereof.

20. The method of claim 19, wherein the IRAK-mediated disorder, disease or condition is selected from the group consisting of a cancer, a neurodegenerative disease, a viral disease, an autoimmune disease, an inflammatory disorder, a hereditary disorder, a hormone-related disease, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, and a CNS disorder.

21. The method of claim 20, wherein the cancer or proliferative disorder is selected from the group consisting of a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastoma, neuroblastoma, multiple myeloma, gastrointestinal cancer, colon carcinoma, colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphoma, Hodgkin's or Non-Hodgkin's lymphoma, a mammary carcinoma, follicular carcinoma, undifferentiated carcinoma, papillary carcinoma, seminoma, melanoma, an IL-1 driven disorder, a MyD88 driven disorder, smoldering or indolent multiple myeloma, and a hematological malignancy selected from leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, or intravascular large B-cell lymphoma.

22. The method of claim 21, wherein the MyD88 driven disorder is selected from the group consisting of ABC DLBCL, Waldenstrom's macroglobulinemia, Hodgkin's lymphoma, primary cutaneous T-cell lymphoma, and chronic lymphocytic leukemia.

23. The method of claim 21, wherein the IL-1 driven disorder is smoldering or indolent multiple myeloma.

24. The method of claim 20, wherein the neurodegenerative disease is selected from the group consisting of Alzheimer's disease, Parkinson's disease, amyotrophic lateral sclerosis, Huntington's disease, cerebral ischemia, and a neurodegenerative disease caused by traumatic injury, glutamate neurotoxicity, hypoxia, epilepsy, treatment of diabetes, metabolic syndrome, obesity, organ transplantation and graft versus host disease.

25. The method of claim 20, wherein the inflammatory disorder is selected from the group consisting of ocular allergy, conjunctivitis, keratoconjunctivitis sicca, vernal conjunctivitis; allergic rhinitis, hemolytic anemia, aplastic anemia, pure red cell anemia, idiopathic thrombocytopenia or another inflammatory disease in which autoimmune reactions are implicated or which have an autoimmune component or etiology, systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, ulcerative colitis, Crohn's disease or another autoimmune inflammatory bowel disease, irritable bowel syndrome, celiac disease, periodontitis, hyaline membrane disease, kidney disease, glomerular disease, alcoholic liver disease, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, vernal keratoconjunctivitis, interstitial lung fibrosis, psoriatic arthritis, systemic juvenile idiopathic arthritis, nephritis, diverticulitis, interstitial cystitis, glomerulonephritis (with and without nephrotic syndrome, optionally including idiopathic nephrotic syndrome or minal change nephropathy), chronic granulomatous disease, endometriosis, leptospiriosis renal disease, glaucoma, retinal disease, aging, headache, pain, complex regional pain syndrome, cardiac hypertrophy, muscle wasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic, non-allergic, mild, moderate, severe, bronchitic, or exercise-induced), acute lung injury, acute respiratory distress syndrome, eosinophilia, hypersensitivities, anaphylaxis, nasal sinusitis, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, Type 2 diabetes, appendicitis, atopic dermatitis, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonia, polymyositis, proctitis, prostatitis, pyelonephritis, rhinitis, salpingitis, sinusitis, stomatitis, synovitis, tendonitis, tonsillitis, vaginitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, juvenile rheumatoid arthritis, Cryopyrin Associated Periodic Syndrome (CAPS), and osteoarthritis.
